(19) United States
(12) Patent Application Publication
(10) Pub. No.: US 2002/0172670 A1
(43) Pub. Date: Nov. 21, 2002

(54) Mannosidase structures

(76) Inventors: David Richard Rose, Markham (CA); Douglas Arthur Kuntz, Toronto (CA); Jean Maria Hubertus Van Den Elsen, Bath (GB)

Correspondence Address: MERCHANT & GOULD PC, P.O. BOX 2903, MINNEAPOLIS, MN, 55402-0903, US

(21) Appl. No.: 09/960,226

(22) Filed: Sep. 21, 2001

Related U.S. Application Data
(60) Provisional application No. 60/263,458, filed on Jan. 23, 2001.
(60) Provisional application No. 60/234,879, filed on Sep. 22, 2000.

(51) Int. Cl. C12N 9/24; A61K 38/47; G06F 19/00; G01N 33/48; G01N 33/50

(52) U.S. Cl. 424/94.61; 435/200; 702/19

(57) ABSTRACT

The present invention relates to a crystal comprising a mannosidase II ligand-binding domain. In particular the present invention relates to a crystal comprising mannosidase II (with and without swainsonine), and its use to generate models for elucidating the structure of other polypeptides and for better identifying ligands capable of modulating mannosidase II activity.

A portion of the disclosure of this patent document contains material that is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to crystal structures. In particular, the invention relates to crystals comprising a mannosidase II ligand binding domain (LBD), optionally having a ligand which is associated therewith. The structures may be used to determine mannosidase homologues and information about the secondary and tertiary structures of polypeptides which are as yet structurally uncharacterised. The structures may also be used to identify ligands which are capable of binding the ligand binding domain. Such ligands may be capable of acting as modulators of mannosidase II activity.

### BACKGROUND

Mannosidase II enzymes

There has been widespread interest in mannosidases in recent years, largely due to their role in a multitude of biological systems and, as a result, their potential as therapeutic targets. In particular, mammalian Golgi α-mannosidase II is involved in glycoprotein biosynthesis (especially in the maturation of N-linked oligosaccharides on newly synthesized glycoproteins) and is currently an important therapeutic target for the development of anti-cancer agents (Goss et al (1995) Clin. Cancer Res. 1:935-944).

Golgi α-mannosidase II (mannosyl oligosaccharide 1,3-1,6-α-mannosidase II, EC 3.2.1.114; also referred to herein as “GMII”) belongs to the glycosyl hydrolase family 38 (Henrissat, 1991; Coutinho and Henrissat, 1999) and is central to the Golgi processing pathway, as it specifically trims two mannose residues from the branched GlcNAcMan₅GlcNAc₂ mannose intermediate (FIG. 8A) to form the core GlcNAcMan₃GlcNAc₂ glycosyl structure, an essential precursor for the further addition of N-acetyl-glucosamine units. GMII is a Type II transmembrane protein, approximately 125 kD in size, composed of a short N-terminal cytoplasmic tail, a single-span transmembrane domain and a large lumenal C-terminal catalytic portion (Moremen and Touster, 1985, 1986). The enzyme is highly specific for the presence of the single GlcNAc attached in a α1,2 linkage to the Man α1,3-Man arm of the GlcNAcMan₅GlcNAc₂-Asn-X substrate (Harpaz and Schachter, 1980). It removes the di-mannose branch (M6, M7; FIG. 8A) by hydrolysis of both glycosidic bonds with net retention of sugar anomeric configuration, resulting in the final tri-mannose GlcNAcMan₃GlcNAc₂ core. There is little or no experimental evidence to date addressing whether the two bonds are cleaved in separate binding sites or sequentially in the same binding site, nor whether or not the singly-hydrolyzed product is released from the enzyme between the two cleavage events.

Mammalian lysosomal-mannosidase has significant sequence similarity to the GM II enzyme and is responsible for glycoprotein degradation (Moremen et al (1994) Glycobiology 4 113-125; Liao et al (1996) J. Biol. Chem. 271:28348-28358). In particular, lysosomal α-mannosidase II is involved in the catabolism of N-linked glycoproteins through the sequential degradation of high mannose, hybrid and complex oligosaccharides.

Mutations in the gene encoding mannosidase II cause α-mannosidosis, an autosomal recessive lysosomal storage disease (Ockermann (1967) Lancet 2:239-241).

A number of mannosidase II genes have been characterised from different sources, including the Drosophila gene (Foster et al (1995) Gene 154:183-186; Rabouille et al (1999) J. Cell Sci. 112:3319-3330), rat gene (Spiro et al (1997) J. Biol. Chem. 272:29356-29363) and human, mouse, bovine and feline genes (Beccari et al (1999) Bioscience reports 19:158-162). These mannosidases have been categorized as class II mannosidases, based on sequence alignment, and belong to family 38 in Henrissat's glycosidase classification (Moremen et al (1994) as above, Henrissat and Bairoch (1996) Biochem J. 316:695-696).

To date there have been significant problems with high level expression of these enzymes, which has impeded structural and mechanistic studies. Indeed, problems with expression have meant that α-mannosidase from Jack Bean (Canavalia ensiformis) has been used as a model enzyme for structural and functional characterisation (Howard et al (1998) J. Biol. Chem. 273:2067-2072; Kimura et al (1999) Eur. J. Biochem. 164:168-175). In view of the potential therapeutic application of mannosidase inhibitors, there is a need for direct structural characterisation of these enzymes.

Swainsonine

Swainsonine (SW) is an indolizidine alkaloid found in Australian Swainsona canescens (Colegate etal., Aust J Chem 32:2257-2264, 1979), North American plants of the genera Astragalus and (Molyneux R J and James L F., Science 215:190-191, 1981), and also the fungus Rhizoctonia leguminicola (Schneider et al., Tetrahedron 39;29-31, 1983).

Swainsonine is a potent and specific inhibitor of the lysosomal and golgi forms of alpha-mannosidase (Cenci di Bello et al., Biochem. J. 215, 693 (1983); Tulsiani et al., J. Biol. Chem. 257, 7936 (1982)). It has potential therapeutic value as an antimetastatic (Humpheries et al., Cancer Res. 48, 1410 (1988)), and tumor-proliferative (Dennis, Cancer Res. 46, 5131 (1986)), or immunoregulatory agent (Kino et al., J. Antibiot. 38, 936 (1985)). Swainsonine has also been shown to have positive effects on cellular immunity in mice (reviewed in Humphries M. J. and Olden K., Pharmacol Ther. 44:85-105, 1989, and Olden et al., Pharmacol Ther 50:285-290, 1991)).

Structural information about the interaction between swainsonine and mannosidase II enzymes would provide a basis for rational modification of swainsonine derivatives with altered activities. It would also provide a framework on which new ligands could be designed which mimic some of the swainsonine:mannosidase atomic interactions.

### SUMMARY OF THE INVENTION

The present invention is based on the finding that, after extensive modifications to the protocol, it is possible to express mannosidase II in appreciable quantities. The present invention is also based on the finding that it is possible to crystallize the protein mannosidase II, both alone and in combination with a selection of different ligands. More particularly, it has been possible to identify the specific sites of mannosidase II which are associated with binding to swainsonine and the mannose-like compound deoxymannojirimycin (DMNJ). The structure was also shown to exhibit a previously unobserved folding pattern enabling the design of novel GMII-specific inhibitors.

Binding domains are of significant utility in drug discovery. The association of natural ligands and substrates with the binding domains of mannosidases is the basis of many biological mechanisms. In addition, many drugs (e.g. swainsonine) exert their effects through association with the binding domains of mannosidases. The associations may occur with all or any parts of a binding domain. An understanding of these associations will lead to the design and optimization of drugs having more favorable associations with their target enzyme and thus provide improved biological effects. Therefore, information about the shape and structure of mannosidases and their ligand-binding domains is invaluable in designing potential modulators of mannosidases for use in treating diseases and conditions associated with or modulated by the mannosidases.

Thus, according to a first aspect of the invention, there is provided a crystal comprising a mannosidase II ligand-binding domain. In a preferred embodiment the crystal is a crystal of a mannosidase II enzyme. The structure of a crystal of mannosidase II has been solved and is set forth in Table 1, Table 2, or Table 8.

The crystal may comprise a complex between a mannosidase II ligand-binding domain and at least one ligand, for example an inhibitor of mannosidase II. In a particularly preferred embodiment that crystal comprises a complex between mannosidase II and swainsonine. The structure of a crystal of a complex between mannosidase II and swainsonine has been solved, and is set forth in Table 2 or Table 8.

In a second aspect, the present invention provides a crystal comprising swainsonine or a derivative thereof. In a preferred embodiment, the crystal comprises a complex between swainsonine (or a derivative thereof) and a mannosidase II ligand-binding domain. The structure of a crystal of a complex between mannosidase II and swainsonine has been solved, and is set forth in Table 2, or Table 8.

According to a third aspect of the invention, there is provided a model of at least part of a mannosidase II, made using a crystal according to the first aspect of the invention. In a preferred embodiment, the model comprises the mannosidase II ligand-binding domain. There is also provided a model of swainsonine or a derivative thereof made using a crystal according to the second aspect of the invention.

The crystal of the first and second aspect of the invention and a model of the third aspect of the invention may be provided in the form of a computer readable medium.

The crystals and models of earlier aspects of the invention may provide information about the atomic contacts involved in the interaction between the enzyme and a known ligand, which can be used to screen for unknown ligands. According to a fourth aspect of the invention, there is provided a method of screening for a ligand capable of binding a mannosidase II ligand binding domain, comprising the use of a crystal according to the first or second aspects of the invention or a model according to the third aspect of the invention. For example, the method may comprise the step of contacting the ligand binding domain with a test compound, and determining if said test compound binds to said ligand binding domain.

In a fifth aspect, the present invention provides a ligand identified by a screening method of the fourth aspect of the invention. Preferably the ligand is a modulator that is capable of modulating the activity of a mannosidase II enzyme.

A crystal and/or model of the invention may be used to design, evaluate, and identity modulators of a mannosidase II or homologues thereof other than ligands that associate with a mannosidase II. The modulators may be based on the shape and structure of a mannosidase II, or a ligand binding domain or atomic interaction, or atomic contacts thereof. Therefore modulators may be derived from ligand binding domains or analogues or parts thereof.

Modulators (e.g. ligands) which are capable of modulating the activity of mannosidase II enzymes have considerable therapeutic and prophylactic potential. In a sixth aspect, the present invention provides the use of a modulator of the invention in the manufacture of a medicament to treat and/or prevent a disease in a mammalian patient. There is also provided a pharmaceutical composition comprising a modulator and a method of treating and/or preventing a disease comprising the step of administering such a modulator or pharmaceutical composition to a mammalian patient.

A potential modulator of a mannosidase II identified by a method of the present invention may be confirmed as a modulator by synthesizing the compound, and testing its effect on the enzymatic activity of mannosidase II in an assay. Such assays are known in the art.

Therefore, the methods of the invention for identifying ligands or modulators may comprise one or more of the following additional steps:

(a) testing whether the modulator or ligand is a modulator of the activity of a mannosidase II, preferably testing the activity of the modulator or ligand in cellular assays and animal model assays;

(b) modifying the modulator or ligand;

(c) optionally rerunning steps (a) or (b); and

(d) preparing a pharmaceutical composition comprising the modulator or ligand.

Steps (a), (b) (c) and (d) may be carried out in any order, at different points in time, and they need not be sequential.

The crystal structures and models described above also provide information about the secondary and tertiary structure of mannosidase II enzymes. This can be used to gleen structural information about other, previously uncharacterised polypeptides. According to a seventh aspect of the invention there is provided a method of determining the secondary and/or tertiary structures of polypeptides with unknown (or only partially known) structure comprising the step of using such a crystal or model. The polypeptide under investigation is preferably structurally or functionally related to the mannosidase II enzyme. For example, the polypeptide may show a degree of homology over some or all parts of the primary amino acid sequence. Alternatively, the polypeptide may perform an analogous function or be suspected to show a similar catalytic mechanism to the mannosidase II enzyme.

Aspects of the invention are presented in the accompanying claims and in the following description, drawings, and Tables.

### DESCRIPTION OF THE FIGURES AND TABLES

The present invention will now be described only by way of example and with reference to the accompanying figures and tables, wherein:

FIG. 1 shows the active site of mannosidase II.

FIG. 2 shows the secondary structure of Drosophila Golgi α-mannosidase II. Helices are in blue and β sheets are in red.

FIG. 3 shows the Drosophila golgi α-mannosidase II molecule with the colours representing where it is identical to human GMII. The red and blue represent deletions or insertions with respect to the human sequence. The green is a disulphide bond.

FIG. 4 shows the whole Drosophila golgi α-mannosidase II molecule in sticks with residues that are identical in the lysosomal manII as coloured balls (red or blue depending whether they are in the N-terminal or C-terminal part of the molecule).

FIG. 5 shows the active site of a Drospholiga mannosidase.

FIG. 6 shows the DNA sequence of an expressed Drosophila mannosidase.

FIG. 7 shows an alignment of expressed secreted Drosophila mannosidase with human mannosidase.

FIG. 8 shows A). Schematic representation of the high mannose GlcNAcMan₅GlcNAc₂ substrate of dGMII. B) Ribbon representation of the dGMII structure, top-view, C) side-view. The loop formed by residues 527-540 is shown in yellow. All molecular images were prepared using MOLSCRIPT (Kraulis, 1991) and rendered using Raster3D (Merritt and Bacon, 1997)

FIG. 9 shows a molecular surface representation of the convex face (A) and the planar face (B) of the dGMII molecule. Molecular surface images are colored for electrostatic potential (red for negative, blue for positive). C) Molecular surface representation of the planar face of dGMII, colored for homology with the sequence of human Golgi α-mannosidase II (dark-green for identical, light-green for homologous, yellow for similar, and white for different residues). Alignment of human and Drosophila Golgi α-mannosidase II sequences (SwissProt accession numbers Q16706 and Q24451, respectively) was performed using the GAP program of the Wisconsin package (Version 10, Genetics Computer Group) using the default parameters without any manual intervention. The scores were used to colour the molecular surface. All molecular surface images were produced using GRASP (Nicholls et al., 1991).

FIG. 10 shows stereo views of the active site of dGMII with bound Tris (A), DMNJ (B), and swainsonine (C) molecules. The active site zinc ion is shown in turquoise, the bound inhibitor molecules are rendered in gold and water molecules are represented as transparent red spheres. Hydrogen bonds are shown as blue dashed lines.

FIG. 11 shows A) Molecular surface representation of dGMII showing the position of the active site bound Tris molecule and the 2-methyl-2,4-pentanediol (MPD) binding site. B) Molecular surface representation of dGMII with the GlcNAcMan₅GlcNAc₂ substrate modeled into the binding pocket. The substrate molecule is positioned into the binding pocket with α1,6-linked mannose M6 (shown in green) docked into the active site and β1,2-GlcNAc residue G3 (shown in black) placed in the MPD binding site. Individual mannose residues of the substrate are colored according to the coloring scheme used in FIGS. 8A. C) Representation of the sequential trimming of the α1,6 (M6) and α1,3-linked (M7) mannose residues. FIG. 11A was produced using LIGPLOT (Wallace et al., 1995). All molecular surface images were produced using GRASP (Nicholls et al., 1991).

Table I shows the structural coordinates of a Drosophila Golgi α-mannosidase II.

Table 2 shows the structural coordinates of a Drosophila Golgi α-mannosidase II with swainsonine.

Table 3 shows the ligand binding domain (active site) of a mannosidase II.

Table 4 shows the intermolecular contacts of a Drosophila Golgi α-mannosidase II swainsonine complex.

Table 5 shows crystallographic refinement statistics for the native Drosophila Golgi mannosidase II.

Table 6 shows crystallographic refinement statistics for Drosophila Golgi mannosidase II associated with swainsonine.

Table 7 shows a list of Mannosidase II enzymes.

Table 8 shows the structural coordinates of a Drosophila Golgi α-mannosidase II with swainsonine, a zinc ion, Tris molecule and an N-glycan.

Table 9 shows data collection statistics for MAD (Se-Met) of dGMII and native dGMII.

Table 10 shows refinement statistics of dGMII, dGMII-swainsonine complex, and dGMII-DMNJ complex.

In Tables 1, 2, and 8 from the left, the second column identifies the atom number; the third identifies the atom type; the fourth identifies the amino acid type; the sixth identifies the residue number; the seventh identifies the x coordinates; the eighth identifies the y coordinates; the ninth identifies the z coordinates; the tenth identifies the occupancy; and the eleventh identifies the temperature factor.

### DETAILED DESCRIPTION OF THE INVENTION

Unless otherwise indicated, all terms used herein have the same meaning as they would to one skilled in the art of the present invention. Practitioners are particularly directed to Current Protocols in Molecular Biology (Ansubel) for definitions and terms of the art. Abbreviations for amino acid residues are the standard 3-letter and/or 1-letter codes used in the art to refer to one of the 20 common L-amino acids.

In a first aspect, the present invention relates to a crystal comprising a mannosidase II ligand binding domain.

Crystal

As used herein, the term “crystal” means a structure (such as a three dimensional (3D) solid aggregate) in which the plane faces intersect at definite angles and in which there is a regular structure (such as internal structure) of the constituent chemical species. Thus, the term “crystal” can include any one of: a solid physical crystal form such as an experimentally prepared crystal, a crystal structure derivable from the crystal (including secondary and/or tertiary and/or quaternary structural elements), a 2D and/or 3D model based on the crystal structure, a representation thereof such as a schematic representation thereof or a diagrammatic representation thereof, or a data set thereof for a computer.

In one aspect, the crystal is usable in X-ray crystallography techniques. Here, the crystals used can withstand exposure to X-ray beams used to produce a diffraction pattern data necessary to solve the X-ray crystallographic structure. A crystalline form of a mannosidase, may be characterized as being capable of diffracting x-rays in a pattern defined by one of the crystal forms depicted in Blundel et all 976, Protein Crystallography, Academic Press.

A crystal of the invention includes a mannosidase II or part thereof (e.g. ligand binding domain) in association with one or more moieties, including heavy-metal atoms i.e. a derivative crystal, a metal cofactor, or one or more ligands or substrates i.e. a co-crystal.

The term “associate”, “association” or “associating” refers to a condition of proximity between a moiety (i.e. chemical entity or compound or portions or fragments thereof), and a mannosidase II, or parts or fragments thereof (e.g. binding sites or domains). The association may be non-covalent i.e. where the juxtaposition is energetically favoured by for example, hydrogen-bonding, van der Waals, or electrostatic or hydrophobic interactions, or it may be covalent.

The term “heavy-metal atoms” refers to an atom that can be used to solve an x-ray crystallography phase problem, including but not limited to a transition element, a lanthanide metal, or an actinide metal. Lanthanide metals include elements with atomic numbers between 57 and 71, inclusive. Actinide metals include elements with atomic numbers between 89 and 103, inclusive.

Multiwavelength anomalous diffraction (MAD) phasing may be used to solve protein structures using selenomethionyl (SeMet) proteins. Therefore, a complex of the invention may comprise a crystalline mannosidase II or part thereof (e.g. ligand binding domain) with selenium associated with the methionine residues of the protein.

In an embodiment of the invention, a ligand binding domain is in association with a metal cofactor in the crystal. A “metal cofactor” refers to a metal required for mannosidase activity and/or stability. For example, the metal cofactor may be zinc, and other similar atoms or metals. In a preferred embodiment a LBD is in association with Zn²⁺.

A ligand binding domain in a complex with a cofactor preferably comprises one or more of the residues involved in coordination of a Zn²⁺ ion, namely: aspartate residues 92 and 204, and histidines 90 and 471.

The crystal may comprise a complex between a ligand-binding domain and one or more ligands. In other words the ligand binding domain may be associated with one or more ligands in the crystal. The ligand may be any compound which is capable of interacting stably and specifically with the ligand binding domain. The ligand may, for example, be an inhibitor of mannosidase II, including but not limited to swainsonine and the mannose-like compound deoxymannojirimycin (DMNJ).

In a preferred embodiment the ligand associated with said mannosidase II ligand binding domain is swainsonine, or an analogue or derivative thereof. Swainsonine is an indolizidine alkaloid found in a variety of sources (Colegate et al., (1979); Molyneux and James (1981); and Schneider et al. (1983) all as above) which has been known to be an inhibitor of mannosidase II enzymes for some time. Derivatives of swainsonine are also known in the art, for example U.S. Pat. No. 5,962,467, No. 5,650,413, and No. 6,048,870, describe various derivatives of swainsonine, processes for their preparation and their use as therapeutic agents.

In an embodiment a crystal of the invention comprises a ligand binding domain of a mannosidase II in association with swainsonine. These complexes may have the structural coordinates shown in Table 2, or Table 8.

In a second aspect, the present invention also provides a crystal comprising swainsonine or a derivative thereof. Preferably the swainsonine molecule has the three dimensional structure defined by the relevant structural coordinates shown in Table 2, or Table 8.

The crystal may also comprise a complex between mannosidase II (or part thereof) and a substrate, or analogue thereof. The term “substrate” refers to molecules that associate with a mannosidase II as it hydrolyzes linkages between mannose residues. Mannosidases II enzymes release α-D-mannose as a first formed product and they follow a double-displacement mechanism in which a glycosyl-enzyme intermediate is formed and hydrolyzed via oxocarbenium ion-like transition states. The formation of the intermediate is assisted by general acid catalysis from a carboxylic acid located in the active site. The residue also serves as the general base catalyst for the second deglycosylation step. A second carboxylic acid serves as the nucleophile that forms the covalent intermediate. Thus, the substrate molecule may comprise molecules such as the glycosyl moiety that forms an intermediate with the enzyme. (See Howard, S. et al, J. Biol. Chem. (1998) 273. 2067-2072 and references 11, 12, 14, 15, and 16 therein). An analogue of a substrate is one which mimics the substrate binding in the LBD, but which is incapable (or has a significantly reduced capacity) to take part in the catalytic reaction.

A number of substrates for Golgi α-mannosidase II are known including the artificial substrate PNP-mannose (Rabouille et al (1999) as above). Lysosomal mannosidase II is involved in glycoprotein degradation. In particular lysosomal mannosidase II hydrolyses α(1,2) α(1,3) and α(1,6) linkages betwwen mannose residues. Substrates for this enzyme are thought to include high mannose, hybrid and complex oligosaccharides.

In an embodiment, the substrate comprises GlcNAcMan₅GlcNAc₂-Asn-.

A complex may comprise one or more of the intermolecular interactions identified in Table 4. A structure of a complex of the invention may be defined by selected intermolecular contacts, preferably the intermolecular contacts as defined in Table 4.

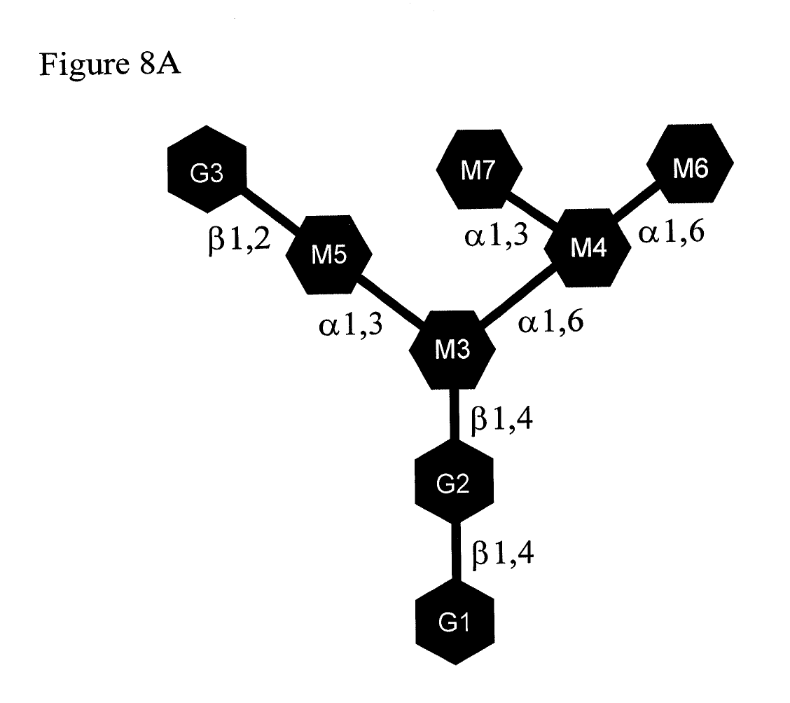

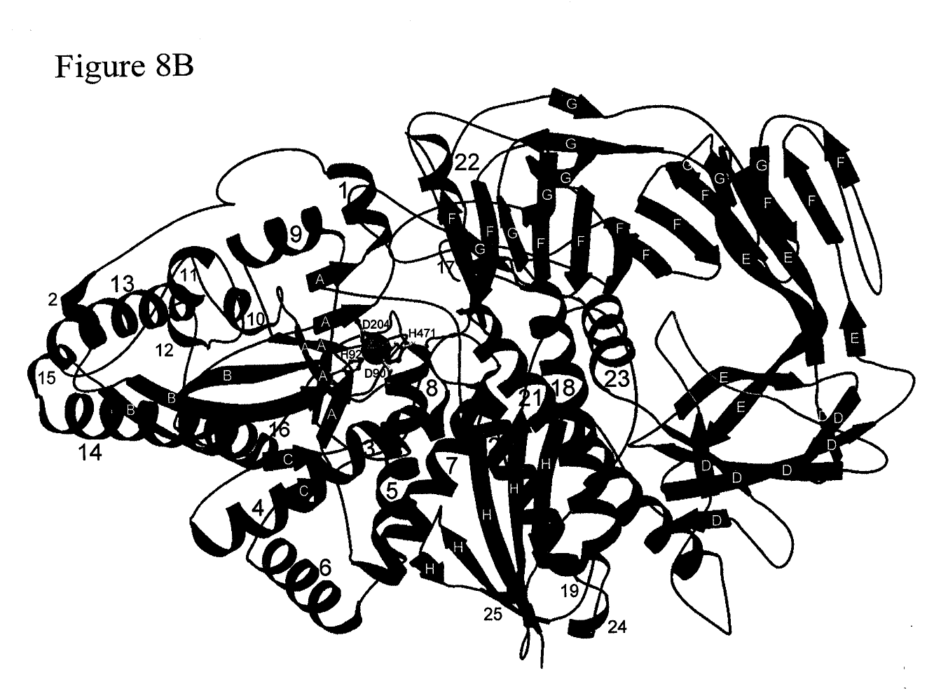

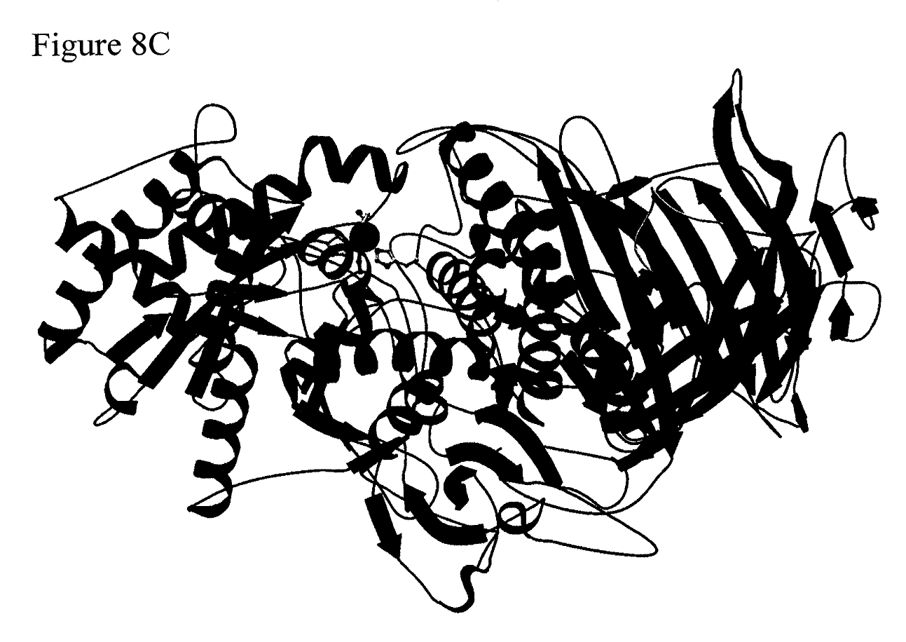

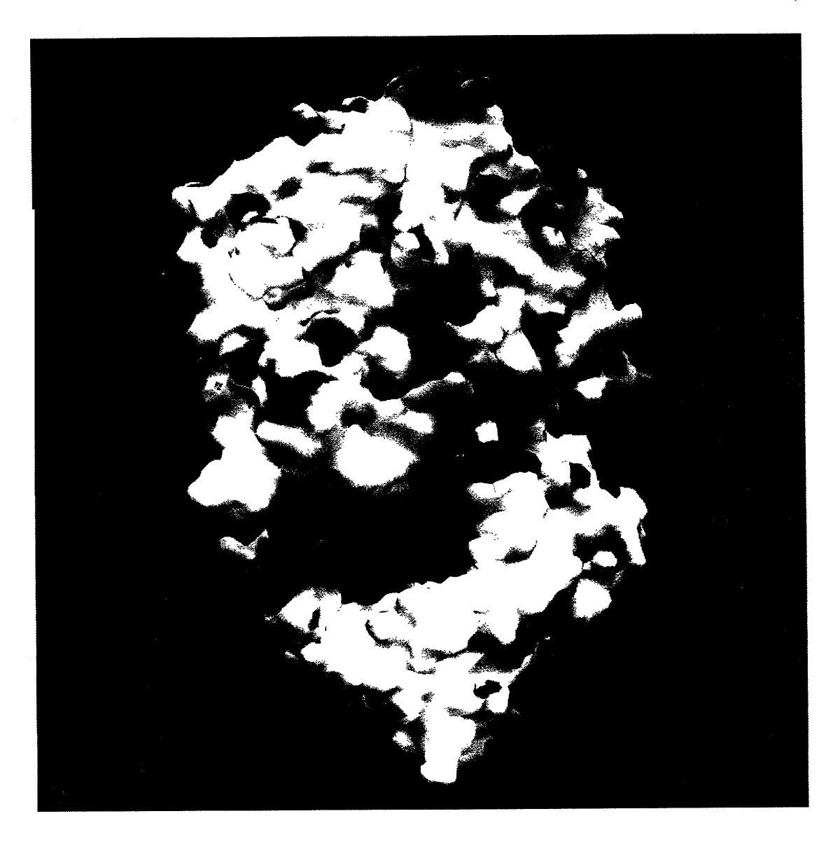

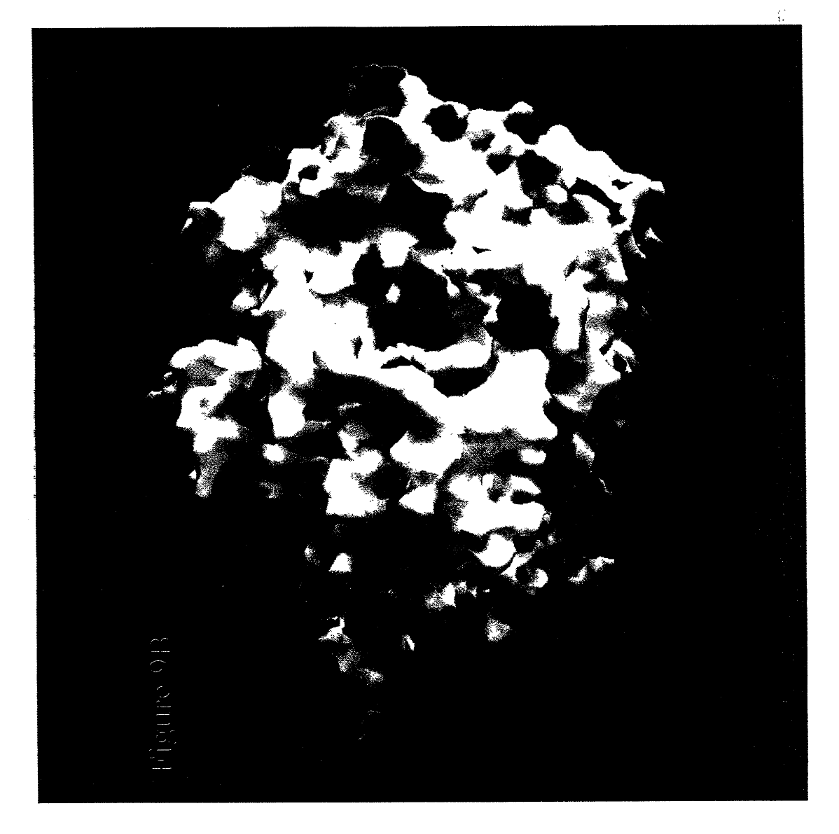

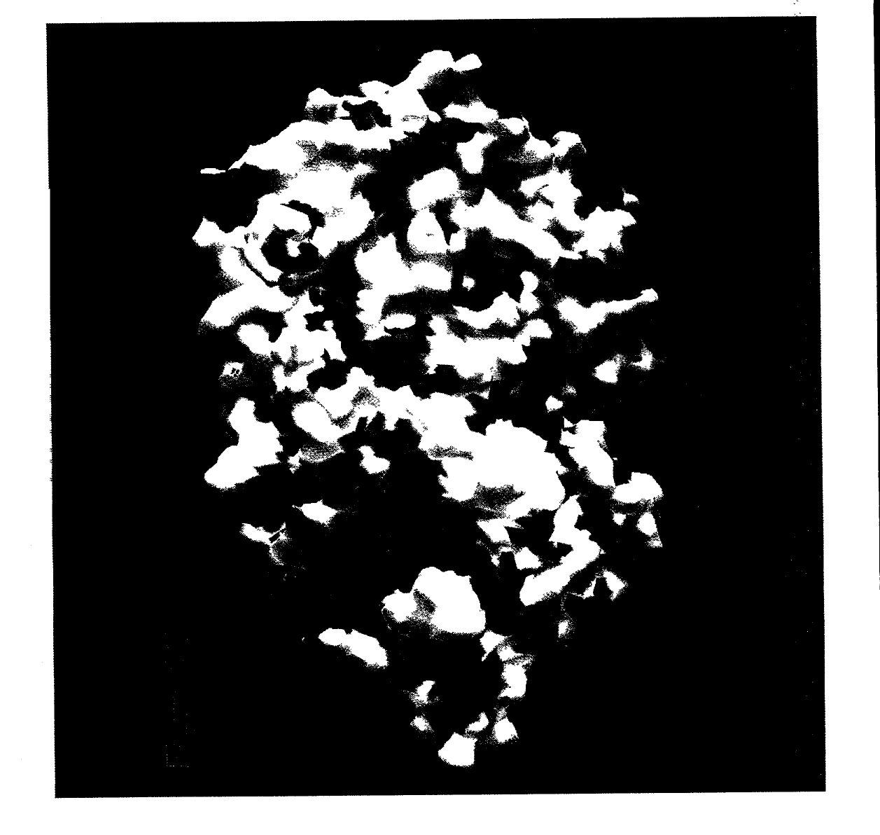

A crystal of the invention may be characterized by an N-terminal α/β domain, a C-terminal portion comprising a three-helical bundle, and an all-β C-terminal domain, connected by 5 internal disulfide bonds and stabilized by a zinc binding site (FIG. 8B).

The N-terminal α/β domain is characterized as follows:

(a) comprising an inner core of three β-sheets (A, B and C, FIG. 8B) consisting of 11, mostly parallel β-strands, surrounded by 16 α-helices;

(b) comprising a GlcNAc residue at a consensus N-glycosylation site (Asn-194), located at the N-terminus of helix 7.

(c) stabilized by three disulfide bonds: between Cys-31 and Cys-1032 connecting the N and C-terminal extremes of dGMII; Cys-275 and Cys-282 linking helices 10 and 11; Cys-283 and Cys-297 linking helix 11 with a loop between helix 13 and the core of parallel β-sheets.

The C-terminal portion is characterized as follows:

(a) a three-helix bundle comprises helices 18, 20 and 21 connected to the N-terminal α/β-domain via a zinc binding site.

(b) a zinc ion coordinated in a T₅-square-based pyramidal geometry involving residues: Asp-90, His-92, Asp-204 and His-471.

(c) two immunoglobulin-like domains: a small β-sandwich consisting of 12 anti-parallel strands from β-sheets D and E, and a large 21-strand structure involving β-sheets F and G.

(d) a barrel formed by the three-helix bundle, helix-23, and the two β-sandwich structures providing a narrow pore in the center of the C-terminal domain.

The barrel in the C-terminal portion is lined by six arginine residues: Arg-540, 565, 617, 770, 777 and 893, contributing to the overall positive charge of the pore (FIG. 9A). A hairpin loop, connecting two strands of β-sheet D (FIGS. 8B and C, residues 527-540, shown in yellow) protrudes into the center of the barrel on the planar side of the molecule. Arginine residue 530, located at the tip of the type-I β-turn in this loop, plugs the pore preventing an open channel through the protein. The resulting crater-like cavity on the convex side of the molecule is 20 Å deep, with a diameter of 20 Å funneling to 8 Å at the bottom of the cavity. The loop has a higher degree of flexibility compared to the rest of the structure (average B-factor values: ˜33 Å² and ˜15 Å², respectively).

A crystal of the invention may enable the determination of structural data for a ligand or substrate. In order to be able to derive structural data for the ligand or substrate, it is necessary for the molecule to have sufficiently strong electron density to enable a model of the molecule to be built using standard techniques. For example, there should be sufficient electron density to allow a model to be built using XTALVIEW (McRee 1992 J. Mol. Graphics. 10 44-46).

Preferably, the crystal of the invention belongs to space group P2₁2₁2₁.

The term “space group” refers to the lattice and symmetry of the crystal. In a space group designation the capital letter indicates the lattice type and the other symbols represent symmetry operations that can be carried out on the contents of the asymmetric unit without changing its appearance.

Preferably, a crystal of said complex comprises a unit cell having the following unit dimensions: a=69 (±5) Å, b=110 (±5) Å, c=139 (±5) Å.

The term “unit cell” refers to the smallest and simplest volume element (i.e. parallelpiped-shaped block) of a crystal that is completely representative of the unit of pattern of the crystal. The unit cell axial lengths are represented by a, b, and c. Those of skill in the art understand that a set of atomic coordinates determined by X-ray crystallography is not without standard error.

In a highly preferred embodiment, the crystal comprises the structural coordinates as shown in Table l, Table 2, or Table 8.

As used herein, the term “structural coordinates” refer to a set of values that define the position of one or more amino acid residues with reference to a system of axes. The term refers to a data set that defines the three dimensional structure of a molecule or molecules (e.g. Cartesian coordinates, temperature factors, and occupancies). Structural coordinates can be slightly modified and still render nearly identical three dimensional structures. A measure of a unique set of structural coordinates is the root-mean-square deviation of the resulting structure. Structural coordinates that render three dimensional structures (in particular a three dimensional structure of an SGC domain) that deviate from one another by a root-mean-square deviation of less than 5 Å, 4 Å, 3 Å, 2 Å, or 1.5 Å may be viewed by a person of ordinary skill in the art as very similar.

Variations in structural coordinates may be generated because of mathematical manipulations of the structural coordinates of a mannosidase described herein. For example, the structural coordinates of Table 1, 2, or 8 may be manipulated by crystallographic permutations of the structural coordinates, fractionalization of the structural coordinates, integer additions or substractions to sets of the structural coordinates, inversion of the structural coordinates or any combination of the above.

Variations in the crystal structure due to mutations, additions, substitutions, and/or deletions of the amino acids, or other changes in any of the components that make up the crystal may also account for modifications in structural coordinates. If such modifications are within an acceptable standard error as compared to the original structural coordinates, the resulting structure may be the same. Therefore, a ligand that bound to a ligand binding domain of a mannosidase would also be expected to bind to another ligand binding domain whose structural coordinates defined a shape that fell within the acceptable error. Such modified structures of a ligand binding domain thereof are also within the scope of the invention.

Various computational analyses may be used to determine whether a molecule or the ligand binding domain thereof is sufficiently similar to all or parts of a ligand binding domain thereof. Such analyses may be carried out using conventional software applications and methods as described herein.

The crystal may also be specifically characterised by the refinement statistics set out in Tables 5, 6, or 10.

Mannosidase II

The term “mannosidase II” refers to eukaryotic mannosidases involved in the biosynthesis of glycoproteins, glycolipids, glycosylphosphatidylinositols and other complex glycoconjugates, and prokaryotic mannosidases involved in the synthesis of carbohydrate structures of bacteria and viruses. In particular, the term refers to the class of mannosidases categorized as class II mannosidases, based on sequence alignment, belonging to family 38 in Henrissat's glycosidase classification (Moremen, K. W. et al (1994) GlycoBiology 4, 113-125; Henrissat, B. and Bairoch A. (1996) Biochem J. 316, 695-696; Henrissat, B. and Bairoch A. (1993) Biochem J. 293, 781-788; Henrissat, B. and Bairoch A. (1991) Biochem J. 280, 309-316). Examples of mannosidase II enzymes include those listed in Table 7 (from http://afmb.cnrs-mrs.fr/˜pedro/CAZY/ghf_{—}38.html).

The invention generally relates to mannosidase II enzymes and parts thereof. Mannosidase II enzymes catalyze the first committed step in the biosynthesis of complex N-glycans and they control conversion of high mannose to complex N-glycans.

Mannosidases are derivable from a variety of sources, including viruses, bacteria, fungi, plants, and animals. In a preferred embodiment the glycosyltransferase is derivable from an animal, preferably a mammal including but not limited to bovine, ovine, porcine, murine equine, most preferably a human. The enzyme may be from any source, whether natural, synthetic, semi-synthetic, or recombinant.

A mannosidase or part thereof in the present invention may be a wild type enzyme, or part thereof, or a mutant, variant or homologue of such an enzyme.

The term “wild type” refers to a polypeptide having a primary amino acid sequence which is identical with the native enzyme (for example, the mammalian enzyme).

The term “mutant” refers to a polypeptide having a primary amino acid sequence which differs from the wild type sequence by one or more amino acid additions, substitutions or deletions. Preferably, the mutant has at least 90% sequence identity with the wild type sequence. Preferably, the mutant has 20 mutations or less over the whole wild-type sequence. More preferably the mutant has 10 mutations or less, most preferably 5 mutations or less over the whole wild-type sequence.

The term “variant” refers to a naturally occurring polypeptide which differs from a wild-type sequence. A variant may be found within the same species (i.e. if there is more than one isoform of the enzyme) or may be found within a different species. Preferably the variant has at least 90% sequence identity with the wild type sequence. Preferably, the variant has 20 mutations or less over the whole wild-type sequence. More preferably, the variant has 10 mutations or less, most preferably 5 mutations or less over the whole wild-type sequence.

The term “part” indicates that the polypeptide comprises a fraction of the wild-type amino acid sequence. It may comprise one or more large contiguous sections of sequence or a plurality of small sections. In an embodiment, the “part” comprises a wild type mannosidase enzyme with the cytosolic and transmembrane domains and most of the stalk region eliminated, preferably the “part” comprises amino acid residues 31-1044 of Golgi α-mannosidase. The “part” may comprise a ligand binding domain as described herein. The polypeptide may also comprise other elements of sequence, for example, it may be a fusion protein with another protein (such as one which aids isolation or crystallisation of the polypeptide). Preferably the polypeptide comprises at least 50%, more preferably at least 65%, most preferably at least 80% of the wild-type sequence.

The term “homologue” means a polypeptide having a degree of homology with the wild-type amino acid sequence. The term “homology” can be equated with “identity”.

In the present context, an homologous sequence is taken to include an amino acid sequence which may be at least 75, 85 or 90% identical, preferably at least 95 or 98% identical to the wild-type sequence. Typically, the homologues will comprise the same sites (for example ligand binding domain) as the subject amino acid sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may have deletions, insertions or substitutions of amino acid residues which produce a silent change and result in a functionally equivalent enzyme. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | |
|---|---|
| ALIPHATIC | |
| Non-polar | G A P |
| | I L V |
| Polar - uncharged | C S T M |
| | N Q |
| Polar - charged | D B |
| | K R |
| AROMATIC | H F W Y |

The polypeptide may also have a homologous substitution (substitution and replacement are both used herein to mean the interchange of an existing amino acid residue, with an alternative residue) i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involving the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell DC, Trends Biotechnol. (1995) 13(4), 132-134.

Ligand-Binding Domain

As used herein, the term “ligand binding domain (LBD)” refers to a region of a molecule or molecular complex that as a result of its shape, favourably associates with a ligand or a part thereof. For example, it may be a region of a mannosidase that is responsible for binding a substrate or modulator (e.g. swainsonine). With reference to the crystal of the present invention residues in the LBD may be defined by their spatial proximity to the ligand (for example swainsonine or substrate) in the crystal structure.

“Ligand” refers to a compound or entity that associates with a ligand binding domain, including substrates or analogues or parts thereof, or modulators of a mannosidase including inhibitors. A ligand may be designed rationally by using a model according to the present invention.

The term “ligand binding domain (LBD)” also includes a homologue of the ligand binding domain or a portion thereof.

As used herein, the term “homologue” in reference to a ligand binding domain refers to ligand binding domain or a portion thereof which may have deletions, insertions or substitutions of amino acid residues as long as the binding specificity of the molecule is retained. In this regard, deliberate amino acid substitutions may be made on the basis of similarity in polarity, charge, solubility, hydrophobicity, hydrophilicity, and/or the amphipathic nature of the residues as long as the binding specificity of the ligand binding domain is retained.

As used herein, the term “portion thereof” means the structural coordinates corresponding to a sufficient number of amino acid residues of the mannosidase II LBD (or homologues thereof) that are capable of interacting with a test compound capable of binding to the LBD. This term includes mannosidase II ligand binding domain amino acid residues having an amino acid residues from about 4 Å to about 5 Å of a bound compound or fragment thereof. Thus, for example, the structural coordinates provided in the crystal structure may contain a subset of the amino acid residues in the LBD which may be useful in the modelling and design of compounds that bind to the LBD.

A ligand binding domain may be defined by its association with a ligand. With reference to a crystal of the present invention, residues in the LBD may be defined by their spatial proximity to a ligand in the crystal structure. For example, such may be defined by their proximity to a substrate or modulator (e.g. swainsonine).

The active site of a mannosidase II crystal of the invention may be characterized as follows:

(a) a small cavity lined by aromatic residues Trp-95, Phe-206, Tyr-269 and Tyr-727;

(b) a zinc ion binding site within the cavity characterized by a T₅-square-based pyramidal geometry and ‘elec-His-Zn motifs’.

A binding domain for a GMII inhibitor such as swainsonine and DMNJ, comprises one or more of Trp-95, Phe-206 and Tyr-727 which form a binding cavity for the inhibitor. The inhibitor ring structures can be stacked against Trp-95, and stabilized by hydrogen bonds and interactions with the zinc ion. When bound to an inhibitor the zinc ion binding domain of the GMII can be transformed into T₆-octahedral coordination. The binding domain allows for the formation of a hydrogen bond between the zinc-coordinating OD 1 oxygen of Asp-204 and the N4 nitrogen at the fusion of the five and six-membered rings of swainsonine. The zinc coordinating oxygen atoms of the inhibitors are involved in hydrogen bond interactions with the neighboring metal binding residues of the enzyme.

The position of the inhibitor molecules is stabilized in the active site by hydrogen bonds between carboxylic oxygens OD1 and OD2 of residue Asp-472 and hydroxyl oxygens O3 and O4 (O5 in swainsonine) of the inhibitors. DMNJ is involved in additional hydrogen bonds, via water molecules, with the NH₂ nitrogen of Arg-228, the hydroxyl oxygen of Tyr-269, the backbone carbonyl oxygen of Arg-876, and the OD1 oxygen of Asp-204.

In an embodiment, a ligand binding domain comprises one or more of the following amino acid residues: His 471, His 90, and Asp 92, and Asp 204; or a homologue thereof.

In a second embodiment, a ligand binding domain comprises one or more of the following amino acid residues: Trp-95, Phe-206, Tyr-269, and Tyr-727.

In another embodiment, a ligand binding domain comprises one or more of the following amino acid residues: Asp-92, Asp-204, His-90, His-471.

In still another embodiment, a ligand binding domain comprises one or more of the following amino acid residues: His 471, Asp 204, Asp 341, His 90, Asp 92, Asp 472, Phe 206, Tyr 727 and Trp 95; or a homologue thereof

In yet another embodiment a ligand binding domain comprises one or more of the following groups:

| | |
|---|---|
| (a) | GVWKQG (residues 60-65) |
| (b) | VFVVPHSHND (residues 83-92) |
| (c) | WAIDPFGH (residues 201-208) |
| (d) | HMMPFYSYDIPHTCGPDPKV/ICCQFDFKR (residues 262-289) |
| (e) | LLI/APLGDDFR (residues 334-343): |

In an aspect of the invention, a ligand binding domain comprises one or more of the enzyme residues shown in Table 3 and/or Table 4.

A crystal of a binding domain may be defined by selected atomic contacts.

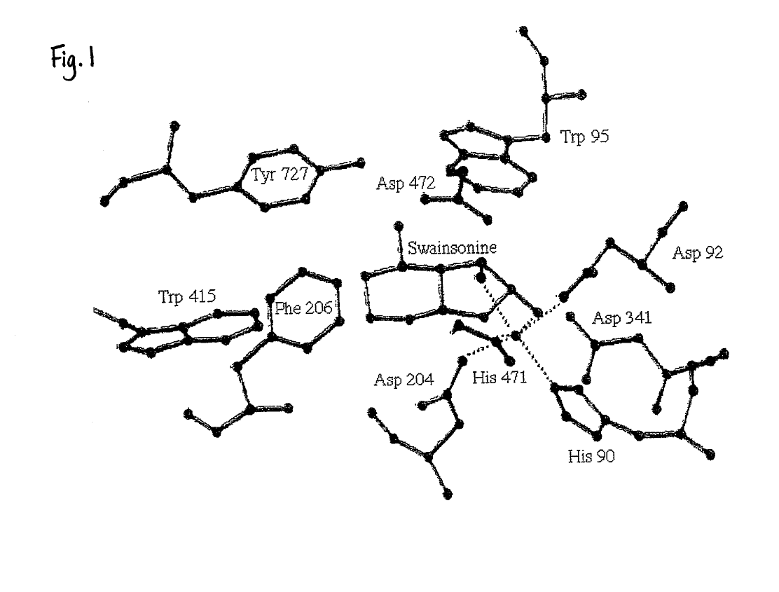

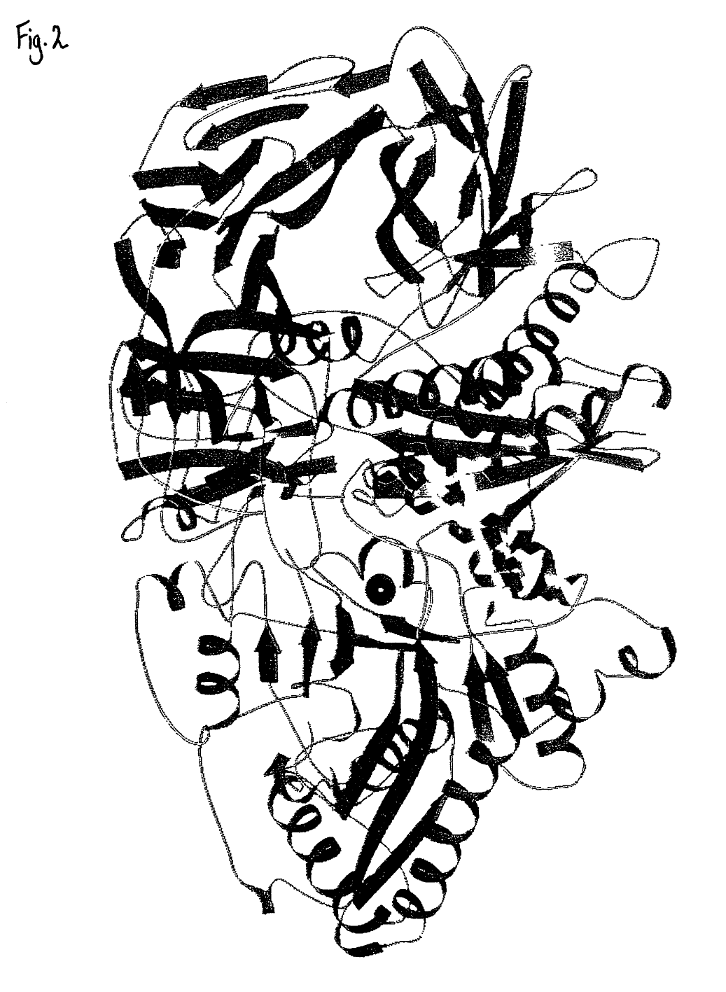

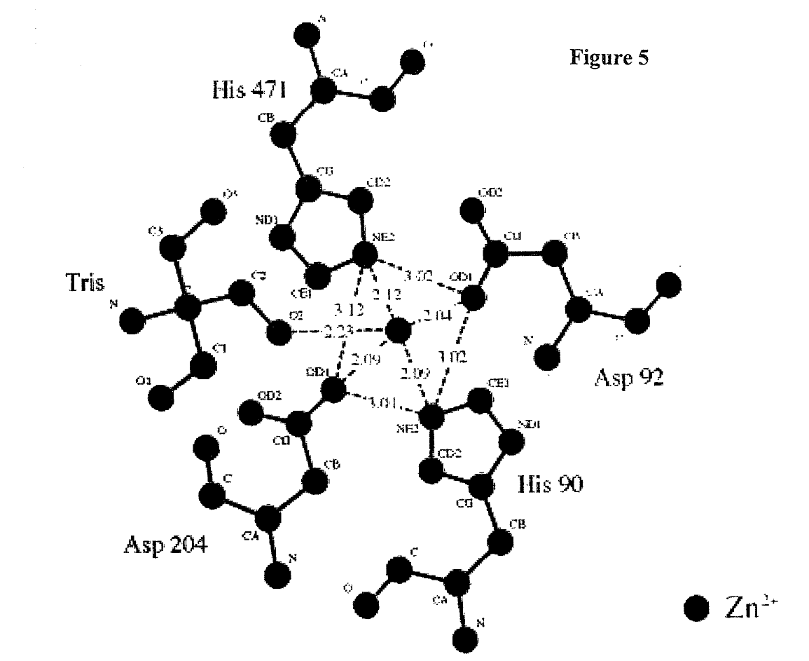

In an embodiment, the binding site of the mannosidase II inhibitor swainsonine is described in Table 3, and details of the atomic interactions of the binding site are set out in Table 4. In the swainsonine binding site there are direct hydrogen bonds between the inhibitor and the enzyme. Atomic contacts on the enzyme comprise Trp-95, Phe-206, Tyr-727, Asp-472, Asp 204 (see Table 4, FIGS. 1 and 5).

In a particular embodiment of the invention, a secondary or three-dimensional structure of a binding domain of a mannosidase II that associates with an inhibitor of a mannosidase II is provided comprising at least two or three atomic contacts of the atomic interactions in Table 4, each atomic interaction defined therein by an atomic contact (more preferably, a specific atom where indicated) on the inhibitor, and an atomic contact (more preferably, a specific amino acid residue where indicated) on the mannosidase II (i.e. enzyme atomic contact). Preferably, the binding domain is defined by the atoms of the enzyme atomic contacts having the structural coordinates for the atoms listed in Table 1, 2, or 8.

Method of Making a Crystal

The present invention also provides a method of making a crystal according to the invention. The crystal may be formed from an aqueous solution comprising a purified polypeptide comprising a mannosidase II or part or fragment thereof (e.g. a catalytic portion, ligand binding domain). A method may utilize a purified polypeptide comprising a mannosidase II ligand binding domain to form a crystal

The term “purified” in reference to a polypeptide, does not require absolute purity such as a homogenous preparation rather it represents an indication that the polypeptide is relatively purer than in the natural environment. Generally, a purified polypeptide is substantially free of other proteins, lipids, carbohydrates, or other materials with which it is naturally associated, preferably at a functionally significant level for example at least 85% pure, more preferably at least 95% pure, most preferably at least 99% pure. A skilled artisan can purify a polypeptide comprising a mannosidase II using standard techniques for protein purification. A substantially pure polypeptide comprising a mannosidase II will yield a single major band on a non-reducing polyacrylamide gel. The purity of the mannosidase II can also be determined by amino-terminal amino acid sequence analysis.

A polypeptide used in the method may be chemically synthesized in whole or in part using techniques that are well-known in the art. Alternatively, methods are well known to the skilled artisan to construct expression vectors containing the native or mutated mannosidase II coding sequence and appropriate transcriptional/translational control signals. These methods include in vitro recombinant DNA techniques, synthetic techniques, and in vivo recombination/genetic recombination. See for example the techniques described in Sambrook et al. (Molecular Cloning: A Laboratory Manual, 2nd Edition, Cold Spring Harbor Laboratory press (1989)), and other laboratory textbooks. (See also Sarker et al, Glycoconjugate J. 7:380, 1990; Sarker et al, Proc. Natl. Acad, Sci. USA 88:234-238, 1991, Sarker et al, Glycoconjugate J. 11: 204-209, 1994; Hull et al, Biochem Biophys Res Commun 176:608, 1991 and Pownall et al, Genomics 12:699-704, 1992).

Crystals may be grown from an aqueous solution containing the purified mannosidase II polypeptide by a variety of conventional processes. These processes include batch, liquid, bridge, dialysis, vapor diffusion, and hanging drop methods. (See for example, McPherson, 1982 John Wiley, New York; McPherson, 1990, Eur. J. Biochem. 189: 1-23; Webber, 1991, Adv. Protein Chem. 41:1-36). Generally, the native crystals of the invention are grown by adding precipitants to the concentrated solution of the mannosidase II polypeptide. The precipitants are added at a concentration just below that necessary to precipitate the protein. Water is removed by controlled evaporation to produce precipitating conditions, which are maintained until crystal growth ceases.

Derivative crystals of the invention can be obtained by soaking native crystals in a solution containing salts of heavy metal atoms. A complex of the invention can be obtained by soaking a native crystal in a solution containing a compound that binds the polypeptide, or they can be obtained by co-crystallizing the polypeptide in the presence of one or more compounds. In order to obtain co-crystals with a compound which binds deep within the tertiary structure of the polypeptide it is necessary to use the second method.

Once the crystal is grown it can be placed in a glass capillary tube and mounted onto a holding device connected to an X-ray generator and an X-ray detection device. Collection of X-ray diffraction patterns are well documented by those skilled in the art (See for example, Ducruix and Geige, 1992, IRL Press, Oxford, England). A beam of X-rays enter the crystal and diffract from the crystal. An X-ray detection device can be utilized to record the diffraction patterns emanating from the crystal. Suitable devices include the Marr 345 imaging plate detector system with an RU200 rotating anode generator.

Methods for obtaining the three dimensional structure of the crystalline form of a molecule or complex are described herein and known to those skilled in the art (see Ducruix and Geige 1992, IRL Press, Oxford, England). Generally, the x-ray crystal structure is given by the diffraction patterns. Each diffraction pattern reflection is characterized as a vector and the data collected at this stage determines the amplitude of each vector. The phases of the vectors may be determined by the isomorphous replacement method where heavy atoms soaked into the crystal are used as reference points in the X-ray analysis (see for example, Otwinowski, 1991, Daresbury, United Kingdom, 80-86). The phases of the vectors may also be determined by molecular replacement (see for example, Naraza, 1994, Proteins 11:281-296). The amplitudes and phases of vectors from the crystalline form of a mannosidase II determined in accordance with these methods can be used to analyze other related crystalline polypeptides.

The unit cell dimensions and symmetry, and vector amplitude and phase information can be used in a Fourier transform function to calculate the electron density in the unit cell i.e. to generate an experimental electron density map. This may be accomplished using the PHASES package (Furey, 1990). Amino acid sequence structures are fit to the experimental electron density map (i.e. model building) using computer programs (e.g. Jones, T A. et al, Acta Crystallogr A47, 100-119, 1991). This structure can also be used to calculate a theoretical electron density map. The theoretical and experimental electron density maps can be compared and the agreement between the maps can be described by a parameter referred to as R-factor. A high degree of overlap in the maps is represented by a low value R-factor. The R-factor can be minimized by using computer programs that refine the structure to achieve agreement between the theoretical and observed electron density map. For example, the XPLOR program, developed by Brunger (1992, Nature 355:472-475) can be used for model refinement.

A three dimensional structure of a molecule or complex may be described by atoms that fit the theoretical electron density characterized by a minimum R value. Files can be created for the structure that defines each atom by coordinates in three dimensions.

Model

A crystal structure of the present invention may be used to make a model of the mannosidase II or a part thereof, (e.g. a ligand-binding domain). A model may, for example, be a structural model (or a representation thereof), or a computer model. A model may represent the secondary, tertiary and/or quaternary structure of the mannosidase II. The model itself may be in two or three dimensions. It is possible for a computer model to be in three dimensions despite the constraints imposed by a conventional computer screen, if it is possible to scroll along at least a pair of axes, causing “rotation” of the image.

Thus, for example, the structural coordinates provided in the crystal structure and/or model structure may comprise the amino acid residues of the mannosidase II LBD, or a portion of the mannosidase II LBD or a homologue thereof useful in the modelling and design of test compounds capable of binding to the mannosidase II LBD.

As used herein, the term “modelling” includes the quantitative and qualitative analysis of molecular structure and/or function based on atomic structural information and interaction models. The term “modelling” includes conventional numeric-based molecular dynamic and energy minimization models, interactive computer graphic models, modified molecular mechanics models, distance geometry and other structure-based constraint models.

Preferably, modelling is performed using a computer and may be further optimized using known methods. This is called modelling optimisation.

Overlays and super positioning with a three dimensional model of the mannosidase II LBD, and/or a portion thereof, can also be used for modelling optimisation. Additionally, alignment and/or modelling can be used as a guide for the placement of mutations on the mannosidase II LBD surface to characterise the nature of the site in the context of a cell.

The three dimensional structure of a new crystal may be modelled using molecular replacement. The term “molecular replacement” refers to a method that involves generating a preliminary model of a molecule or complex whose structural coordinates are unknown, by orienting and positioning a molecule whose structural coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115. pp. 55-77 (1985); M. G. Rossmann, ed., “The Molecular Replacement Method”, Int. Sci. Rev. Ser., No. 13, Gordon & Breach, New York, (1972).

Commonly used computer software packages for molecular replacement are X-PLOR (Brunger 1992, Nature 355: 472-475), AMoRE (Navaza, 1994, Acta Crystallogr. A50:157-163), the CCP4 package (Collaborative Computational Project, Number 4, “The CCP4 Suite: Programs for Protein Crystallography”, Acta Cryst., Vol. D50, pp. 760-763, 1994), the MERLOT package (P. M. D. Fitzgerald, J. Appl. Cryst., Vol. 21, pp. 273-278, 1988) and XTALVIEW (McCree et al (1992) J. Mol. Graphics 10: 44-46. It is preferable that the resulting structure not exhibit a root-mean-square deviation of more than 3 Å.

The quality of the model may be analysed using a program such as PROCHECK or 3D-Profiler [Laskowski et al 1993 J. Appl. Cryst. 26:283-291; Luthy R. et al, Nature 356: 83-85, 1992; and Bowie, J. U. et al, Science 253: 164-170, 1991]. Once any irregularities have been resolved, the entire structure may be further refined.

Other molecular modelling techniques may also be employed in accordance with this invention. See, e.g., Cohen, N. C. et al, “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Using the structural coordinates of the crystal complexes provided by this invention, molecular modelling may be used to determine the structure coordinates of a crystalline mutant or homologue of mannosidase II LBD or of a related protein. By the same token, a crystal of the second aspect of the invention can be used to provide a model of swainsonine. Modelling techniques can then be used to approximate the three dimensional structure of swainsonine derivatives and other compounds which may be able to mimic the atomic contacts between swainsonine and the LBD.

Computer Format of Crystals/Models

Information derivable from the crystal of the present invention (for example the structural coordinates) and/or a model of the present invention may be provided in a computer-readable format.

Therefore, the invention provides a computer readable medium or a machine readable storage medium which comprises the structural coordinates of a mannosidase II including all or any parts of the mannosidase II (e.g ligand-binding domain), ligands including portions thereof, or substrates including portions thereof. Such storage medium or storage medium encoded with these data are capable of displaying on a computer screen or similar viewing device, a three-dimensional graphical representation of a molecule or molecular complex which comprises the enzyme or ligand binding domains or similarly shaped homologous enzymes or ligand binding domains. Thus, the invention also provides computerized representations of a crystal of the invention, including any electronic, magnetic, or electromagnetic storage forms of the data needed to define the structures such that the data will be computer readable for purposes of display and/or manipulation.

In an aspect the invention provides a computer for producing a three-dimensional representation of a molecule or molecular complex, wherein said molecule or molecular complex comprises a mannosidase II or ligand binding domain thereof defined by structural coordinates of mannosidase II amino acids or a ligand binding domain thereof, or comprises structural coordinates of atoms of a ligand or substrate, or a three-dimensional representation of a homologue of said molecule or molecular complex, wherein said computer comprises:

(a) a machine-readable data storage medium comprising a data storage material encoded with machine readable data wherein said data comprises the structural coordinates of a mannosidase II amino acids according to Table 1, 2, or 8 or a ligand binding domain thereof, or a ligand (e.g. swainsonine) according to Table 2, or Table 8;

(b) a working memory for storing instructions for processing said machine-readable data;

(c) a central-processing unit coupled to said working memory and to said machine-readable data storage medium for processing said machine readable data into said three-dimensional representation; and

(d) a display coupled to said central-processing unit for displaying said three-dimensional representation.

A homologue may comprise a mannosidase II or ligand binding domain thereof, or ligand or substrate that has a root mean square deviation from the backbone atoms of not more than 1.5 angstroms.

The invention also provides a computer for determining at least a portion of the structural coordinates corresponding to an X-ray diffraction pattern of a molecule or molecular complex wherein said computer comprises:

(a) a machine-readable data storage medium comprising a data storage material encoded with machine readable data wherein said data comprises the structural coordinates according to Table 1, 2, or 8;

(b) a machine-readable data storage medium comprising a data storage material encoded with machine readable data wherein said data comprises an X-ray diffraction pattern of said molecule or molecular complex;

(c) a working memory for storing instructions for processing said machine-readable data of (a) and (b);

(d) a central-processing unit coupled to said working memory and to said machine-readable data storage medium of (a) and (b) for performing a Fourier transform of the machine readable data of (a) and for processing said machine readable data of (b) into structural coordinates; and

(e) a display coupled to said central-processing unit for displaying said structural coordinates of said molecule or molecular complex.

Structural Determinations

The present invention also provides a method for determining the secondary and/or tertiary structures of a polypeptide by using a crystal, or a model according to the present invention. The polypeptide may be any polypeptide for which the secondary and or tertiary structure is uncharacterised or incompletely characterised. In a preferred embodiment the polypeptide shares (or is predicted to share) some structural or functional homology to the mannosidase II crystal. For example, the polypeptide may show a degree of structural homology over some or all parts of the primary amino acid sequence. For example the polypeptide may have one or more domains which shows homology with a mannosidase II domain (Kapitonov and Yu (1999) Glycobiology 9(10): 961-978).

The polypeptide may be a mannosidase II with a different specificity for a ligand or substrate. The polypeptide may be a mannosidase II which requires a different metal cofactor. Alternatively (or in addition) the polypeptide may be a mannosidase II from a different species.

The polypeptide may be a mutant of the wild-type mannosidase II. A mutant may arise naturally, or may be made artificially (for example using molecular biology techniques). The mutant may also not be “made” at all in the conventional sense, but merely tested theoretically using the model of the present invention. A mutant may or may not be functional.

Thus, using the model of the present invention, the effect of a particular mutation on the overall two and/or three dimensional structure of a mannosidase II and/or the interaction between the enzyme and a ligand or substrate can be investigated. Alternatively, the polypeptide may perform an analogous function or be suspected to show a similar catalytic mechanism to the mannosidase II enzyme. For example the polypeptide may remove, transport, or add on a sugar residue.

The polypeptide may also be the same as the polypeptide of the crystal, but in association with a different ligand (for example, modulator or inhibitor) or cofactor. In this way it is possible to investigate the effect of altering a ligand or compound with which the polypeptide is associated on the structure of the LBD.

Secondary or tertiary structure may be determined by applying the structural coordinates of the crystal or model of the present invention to other data such as an amino acid sequence, X-ray crystallographic diffraction data, or nuclear magnetic resonance (NMR) data. Homology modeling, molecular replacement, and nuclear magnetic resonance methods using these other data sets are described below.

Homology modeling (also known as comparative modeling or knowledge-based modeling) methods develop a three dimensional model from a polypeptide sequence based on the structures of known proteins (i.e. mannosidase II of the crystal). The method utilizes a computer model of the crystal of the present invention (the “known structure”), a computer representation of the amino acid sequence of the polypeptide with an unknown structure, and standard computer representations of the structures of amino acids. The method in particular comprises the steps of, (a) identifying structurally conserved and variable regions in the known structure; (b) aligning the amino acid sequences of the known structure and unknown structure (c) generating coordinates of main chain atoms and side chain atoms in structurally conserved and variable regions of the unknown structure based on the coordinates of the known structure thereby obtaining a homology model; and (d) refining the homology model to obtain a three dimensional structure for the unknown structure. This method is well known to those skilled in the art (Greer, 1985, Science 228, 1055; Bundell et al 1988, Eur. J. Biochem. 172, 513; Knighton et al., 1992, Science 258:130-135, http://biochem.vt.edu/courses/modeling/homology.htn). Computer programs that can be used in homology modeling are Quanta and the Homology module in the Insight II modelling package distributed by Molecular Simulations Inc, or MODELLER (Rockefeller University, www.iucr.ac.uk/sinris-top/logical/prg-modeller.html).

In step (a) of the homology modeling method, the known mannosidase II structure is examined to identify the structurally conserved regions (SCRs) from which an average structure, or framework, can be constructed for these regions of the protein. Variable regions (VRs), in which known structures may differ in conformation, also must be identified. SCRs generally correspond to the elements of secondary structure, such as alpha-helices and beta-sheets, and to ligand- and substrate-binding sites (e.g. acceptor and donor binding sites). The VRs usually lie on the surface of the proteins and form the loops where the main chain turns.

Many methods are available for sequence alignment of known structures and unknown structures. Sequence alignments generally are based on the dynamic programming algorithm of Needleman and Wunsch [J. Mol. Biol. 48: 442-453, 1970]. Current methods include FASTA, Smith-Waterman, and BLASTP, with the BLASTP method differing from the other two in not allowing gaps. Scoring of alignments typically involves construction of a 20×20 matrix in which identical amino acids and those of similar character (i.e., conservative substitutions) may be scored higher than those of different character. Substitution schemes which may be used to score alignments include the scoring matrices PAM (Dayhoff et al., Meth. Enzymol. 91: 524-545, 1983), and BLOSUM (Henikoff and Henikoff, Proc. Nat. Acad. Sci. USA 89: 10915-'0919, 1992), and the matrices based on alignments derived from three-dimensional structures including that of Johnson and Overington (JO matrices) (J. Mol. Biol. 233: 716-738, 1993).

Alignment based solely on sequence may be used; however, other structural features also may be taken into account. In Quanta, multiple sequence alignment algorithms are available that may be used when aligning a sequence of the unknown with the known structures. Four scoring systems (i.e. sequence homology, secondary structure homology, residue accessibility homology, CA-CA distance homology) are available, each of which may be evaluated during an alignment so that relative statistical weights may be assigned.

When generating coordinates for the unknown structure, main chain atoms and side chain atoms, both in SCRs and VRs need to be modeled. A variety of approaches known to those skilled in the art may be used to assign coordinates to the unknown. In particular, the coordinates of the main chain atoms of SCRs will be transferred to the unknown structure. VRs correspond most often to the loops on the surface of the polypeptide and if a loop in the known structure is a good model for the unknown, then the main chain coordinates of the known structure may be copied. Side chain coordinates of SCRs and VRs are copied if the residue type in the unknown is identical to or very similar to that in the known structure. For other side chain coordinates, a side chain rotamer library may be used to define the side chain coordinates. When a good model for a loop cannot be found fragment databases may be searched for loops in other proteins that may provide a suitable model for the unknown. If desired, the loop may then be subjected to conformational searching to identify low energy conformers if desired.

Once a homology model has been generated it is analyzed to determine its correctness. A computer program available to assist in this analysis is the Protein Health module in Quanta which provides a variety of tests. Other programs that provide structure analysis along with output include PROCHECK and 3D-Profiler [Luthy R. et al, Nature 356: 83-85, 1992; and Bowie, J. U. et al, Science 253: 164-170, 1991]. Once any irregularities have been resolved, the entire structure may be further refined. Refinement may consist of energy minimization with restraints, especially for the SCRs. Restraints may be gradually removed for subsequent minimizations. Molecular dynamics may also be applied in conjunction with energy minimization.

Molecular replacement involves applying a known structure to solve the X-ray crystallographic data set of a polypeptide of unknown structure. The method can be used to define the phases describing the X-ray diffraction data of a polypeptide of unknown structure when only the amplitudes are known. Thus in an embodiment of the invention, a method is provided for determining three dimensional structures of polypeptides with unknown structure by applying the structural coordinates of the crystal of the present invention to provide an X-ray crystallographic data set for a polypeptide of unknown structure, and (b) determining a low energy conformation of the resulting structure.

Molecular replacement computer programs generally involve the following steps: (1) determining the number of molecules in the unit cell and defining the angles between them (self rotation function); (2) rotating the known structure against diffraction data to define the orientation of the molecules in the unit cell (rotation function); (3) translating the known structure in three dimensions to correctly position the molecules in the unit cell (translation function); (4) determining the phases of the X-ray diffraction data and calculating an R-factor calculated from the reference data set and from the new data wherein an R-factor between 30-50% indicates that the orientations of the atoms in the unit cell have been reasonably determined by the method; and (5) optionally, decreasing the R-factor to about 20% by refining the new electron density map using iterative refinement techniques known to those skilled in the art (refinement).

In an embodiment of the invention, a method is provided for determining three dimensional structures of polypeptides with unknown structure (e.g. additional native or mutated mannosidase II enzymes) by applying the structural coordinates of a mannosidase II structure to provide an X-ray crystallographic data set for a polypeptide of unknown structure, and (b) determining a low energy conformation of the resulting structure.

The structural coordinates of the crystal of the present invention may be applied to nuclear magnetic resonance (NMR) data to determine the three dimensional structures of polypeptides with uncharacterised or incompletely characterised sturcture. (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural coordinates of a polypeptide defined by X-ray crystallography can guide the NMR spectroscopist to an understanding of the spatial interactions between secondary structural elements in a polypeptide of related structure. Information on spatial interactions between secondary structural elements can greatly simplify Nuclear Overhauser Effect (NOE) data from two-dimensional NMR experiments. In addition, applying the structural coordinates after the determination of secondary structure by NMR techniques simplifies the assignment of NOE's relating to particular amino acids in the polypeptide sequence and does not greatly bias the NMR analysis of polypeptide structure.

In an embodiment, the invention relates to a method of determining three dimensional structures of polypeptides with unknown structures, by applying the structural coordinates of a crystal of the present invention to nuclear magnetic resonance (NMR) data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assignment” defines a method of analyzing NMR data and identifying which amino acids give rise to signals in the NMR spectrum.

Screening Method

The present invention also provides a method of screening for a ligand that associates with a ligand binding domain and/or modulates the function of mannosidase II, by using a crystal or a model according to the present invention. The method may involve investigating whether a test compound is capable of associating with or binding a ligand binding domain.

In accordance with an aspect of the present invention, a method is provided for screening for a ligand capable of binding to a ligand binding domain, wherein said method comprises the use of a crystal or model according to the invention.

In another aspect, the invention relates to a method of screening for a ligand capable of binding to a ligand binding domain, wherein the ligand binding domain is defined by the amino acid residue structural coordinates given herein, the method comprising contacting the ligand binding domain with a test compound and determining if said test compound binds to said ligand binding domain.

In one embodiment, the present invention provides a method of screening for a test compound capable of interacting with a key amino acid residue of the ligand binding domain of mannosidase II.

Another aspect of the invention provides a process comprising the steps of:

(a) performing the method of screening for a ligand as described above;

(b) identifying one or more ligands capable of binding to a ligand binding domain; and

(c) preparing a quantity of said one or more ligands.

A further aspect of the invention provides a process comprising the steps of:

(a) performing the method of screening for a ligand as described above;

(b) identifying one or more ligands capable of binding to a ligand binding domain; and

(c) preparing a pharmaceutical composition comprising said one or more ligands.

Once a test compound capable of interacting with a key amino acid residue in a mannosidase II LBD has been identified, further steps may be carried out either to select and/or to modify compounds and/or to modify existing compounds, to modulate the interaction with the key amino acid residues in the mannosidase II LBD.

Yet another aspect of the invention provides a process comprising the steps of:

(a) performing the method of screening for a ligand as described above;

(b) identifying one or more ligands capable of binding to a ligand binding domain;

(c) modifying said one or more ligands capable of binding to a ligand binding domain;

(d) performing said method of screening for a ligand as described above;

(e) optionally preparing a pharmaceutical composition comprising said one or more ligands.

As used herein, the term “test compound” means any compound which is potentially capable of associating with a ligand binding domain. If, after testing, it is determined that the test compound does bind to the LBD, it is known as a “ligand”.

A “test compound” includes, but is not limited to, a compound which may be obtainable from or produced by any suitable source, whether natural or not. The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatised test compound, a peptide cleaved from a whole protein, or a peptides synthesised synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

The test compound may be screened as part of a library or a data base of molecules. Data bases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Test compounds may be tested for their capacity to fit spatially into a mannosidsase II LBD. As used herein, the term “fits spatially” means that the three-dimensional structure of the test compound is accommodated geometrically in a cavity or pocket of the mannosidase II LBD. The test compound can then be considered to be a ligand.

A favourable geometric fit occurs when the surface areas of the test compound is in close proximity with the surface area of the cavity or pocket without forming unfavorable interactions. A favourable complementary interaction occurs where the test compound interacts by hydrophobic, aromatic, ionic, dipolar, or hydrogen donating and accepting forces. Unfavourable interactions may be steric hindrance between atoms in the test compound and atoms in the binding site.

If a model of the present invention is a computer model, the test compounds may be positioned in an LBD through computational docking. If, on the other hand, the model of the present invention is a structural model, the test compounds may be positioned in the LBD by, for example, manual docking.

As used herein the term “docking” refers to a process of placing a compound in close proximity with a mannosidase II LBD, or a process of finding low energy conformations of a test compound/glycosyltransferase complex.

A screening method of the present invention may comprise the following steps:

(i) generating a computer model of a mannosidase II or a selected site thereof using a crystal according to the first aspect of the invention;

(ii) docking a computer representation of a test compound with the computer model;

(iii) analysing the fit of the compound in the mannosidase II or selected site.

In an aspect of the invention a method is provided comprising the following steps:

(a) docking a computer representation of a structure of a test compound into a computer representation of a binding domain of a mannosidase II defined in accordance with the invention using a computer program, or by interactively moving the representation of the test compound into the representation of the binding domain;

(b) characterizing the geometry and the complementary interactions formed between the atoms of the binding domain and the compound; optionally

(c) searching libraries for molecular fragments which can fit into the empty space between the compound and binding domain and can be linked to the compound; and

(d) linking the fragments found in (c) to the compound and evaluating the new modified compound.

In an embodiment of the invention a method is provided which comprises the following steps:

(a) docking a computer representation of a test compound from a computer data base with a computer representation of a selected site (e.g. the inhibitor binding domain) on a mannosidase II structure defined in accordance with the invention to obtain a complex;

(b) determining a conformation of the complex with a favourable geometric fit and favourable complementary interactions; and

(c) identifying test compounds that best fit the selected site as potential modulators of the mannosidase II.

A method of the invention may be applied to a plurality of test compounds, to identify those that best fit the selected site.

The model used in the screening method may comprise the ligand-binding domain of a mannosidase II enzyme either alone or in association with one or more ligands and/or cofactors. For example, the model may comprise the ligand-binding domain in association with a substrate or analogue thereof.

If the model comprises an unassociated ligand binding domain, then the selected site under investigation may be the LBD itself. The test compound may, for example, mimic a known substrate for the enzyme in order to interact with the LBD. The selected site may alternatively be another site on the enzyme.

If the model comprises an associated LBD, for example an LBD in association with a substrate molecule or analogue thereof, the selected site may be the LBD or a site made up of the LBD and the complexed ligand, or a site on the ligand itself. The test compound may be investigated for its capacity to modulate the interaction with the associated molecule.

A test compound (or plurality of test compounds) may be selected on the basis of its similarity to a known ligand for the mannosidase II. For example, the screening method may comprise the following steps:

(i) generating a computer model of the LBD of a mannosidase II in complex with a ligand;

(ii) searching for a test compound with a similar three dimensional structure and/or similar chemical groups; and

(iii) evaluating the fit of the test compound in the LBD.

Searching may be carried out using a database of computer representations of potential compounds, using methods known in the art.

The present invention also provides a method for designing ligands for a mannosidase II. It is well known in the art to use a screening method as described above to identify a test compound with promising fit, but then to use this test compound as a starting point to design a ligand with improved fit to the model. A known modulator can also be modified to enhance its fit with a model of the invention. Such techniques are known as “structure-based ligand design” (See Kuntz et al., 1994, Acc. Chem. Res. 27:117; Guida, 1994, Current Opinion in Struc. Biol. 4: 777; and Colman, 1994, Current Opinion in Struc. Biol. 4: 868, for reviews of structure-based drug design and identification; and Kuntz et al 1982, J. Mol. Biol. 162:269; Kuntz et al., 1994, Acc. Chem. Res. 27: 117; Meng et al., 1992, J. Compt. Chem. 13: 505; Bohm, 1994, J. Comp. Aided Molec. Design 8: 623 for methods of structure-based modulator design).

Examples of computer programs that may be used for structure-based ligand design are CAVEAT (Bartlett et al., 1989, in “Chemical and Biological Problems in Molecular Recognition”, Roberts, S. M. Ley, S. V.; Campbell, N. M. eds; Royal Society of Chemistry: Cambridge, pp 182-196); FLOG (Miller et al., 1994, J. Comp. Aided Molec. Design 8:153); PRO Modulator (Clark et al., 1995 J. Comp. Aided Molec. Design 9:13); MCSS (Miranker and Karplus, 1991, Proteins: Structure, Function, and Genetics 8:195); and, GRID (Goodford, 1985, J. Med. Chem. 28:849).

The method may comprise the following steps:

(i) docking a model of a test compound with a model of a selected site;

(ii) identifying one or more groups on the test compound which may be modified to improve their fit in the selected site;

(iii) replacing one or more identified groups to produce a modified test compound model; and

(iv) docking the modified test compound model with the model of the selected site.

Evaluation of fit may comprise the following steps:

(a) mapping chemical features of a test compound such as by hydrogen bond donors or acceptors, hydrophobic/lipophilic sites positively ionizable sites, or negatively ionizable sites; and

(b) adding geometric constraints to selected mapped features.

The fit of the modified test compound may then be evaluated using the same criteria.

The chemical modification of a group may either enhance or reduce hydrogen bonding interaction, charge interaction, hydrophobic interaction, Van Der Waals interaction or dipole interaction between the test compound and the key amino acid residue(s) of the selected site. Preferably the group modifications involve the addition, removal, or replacement of substituents onto the test compound such that the substituents are positioned to collide or to bind preferentially with one or more amino acid residues that correspond to the key amino acid residues of the selected site.

Identified groups in a test compound may be substituted with, for example, alkyl, alkoxy, hydroxyl, aryl, cycloalkyl, alkenyl, alkynyl, thiol, thioalkyl, thioaryl, amino, or halo groups. Generally, initial substitutions are conservative, i.e., the replacement group will have approximately the same size, shape, hydrophobicity and charge as the original group. It should, of course, be understood that components known in the art to alter conformation should be avoided.

If a modified test compound model has an improved fit, then it may bind to the selected site and be considered to be a “ligand”. Rational modification of groups may be made with the aid of libraries of molecular fragments which may be screened for their capacity to fit into the available space and to interact with the appropriate atoms. Databases of computer representations of libraries of chemical groups are available commercially, for this purpose.

A test compound may also be modified “in situ” (i.e. once docked into the potential binding site), enabling immediate evaluation of the effect of replacing selected groups. The computer representation of the test compound may be modified by deleting a chemical group or groups, replacing chemical groups, or by adding a chemical group or groups. After each modification to a compound, the atoms of the modified compound and potential binding site can be shifted in conformation and the distance between the modulator and the active site atoms may be scored on the basis of geometric fit and favourable complementary interactions between the molecules. This technique is described in detail in Molecular Simulations User Manual, 1995 in LUDI.

Examples of ligand building and/or searching computer include programs in the Molecular Simulations Package (Catalyst), ISIS/HOST, ISIS/BASE, and ISIS/DRAW (Molecular Designs Limited), and UNITY (Tripos Associates).

The “starting point” for rational ligand design may be a known ligand for the enzyme. For example, in order to identify potential modulators of the mannosidase II, a logical approach would be to start with a known ligand (for example a substrate molecule or inhibitor) to produce a molecule which mimics the binding of the ligand. Such a molecule may, for example, act as a competitive inhibitor for the true ligand, or may bind so strongly that the interaction (and inhibition) is effectively irreversible.

Such a method may comprise the following steps:

(i) generating a computer model of a LBD of a mannosidase II in complex with a ligand;

(ii) replacing one or more groups on the ligand model to produce a modified ligand; and

(iii) evaluating the fit of the modified ligand in the LBD.

The replacement groups could be selected and replaced using a compound construction program which replaces computer representations of chemical groups with groups from a computer database, where the representations of the compounds are defined by structural coordinates.

In an embodiment, a screening method is provided for identifying a ligand of a mannosidase II comprising the step of using the structural coordinates of a substrate molecule or swainsonine or component thereof, defined in relation to its spatial association with a mannosidase II structure or a ligand binding domain of the invention, to generate a compound that is capable of associating with the mannosidase II or ligand binding domain.

In an embodiment of the invention, a screening method is provided for identifying a ligand of a mannosidase II comprising the step of using the structural coordinates of swainsonine listed in Table 2 or 8 to generate a compound for associating with a ligand binding domain of a mannosidase II as described herein. The following steps are employed in a particular method of the invention: (a) generating a computer representation of swainsonine, defined by its structural coordinates listed in Table 2 or 8; (b) searching for molecules in a data base that are structurally or chemically similar to the defined swainsonine, using a searching computer program, or replacing portions of the compound with similar chemical structures from a database using a compound building computer program.

The screening methods of the present invention may be used to identify compounds or entities that associate with a molecule that associates with a mannosidase II enzyme (for example, a substrate molecule).

Compounds and entities (e.g. ligands) of mannosidase II identified using the above-described methods may be prepared using methods described in standard reference sources utilized by those skilled in the art. For example, organic compounds may be prepared by organic synthetic methods described in references such as March, 1994, Advanced Organic Chemistry: Reactions, Mechanisms, and Structure, New York, McGraw Hill.

Test compounds and ligands which are identified using a crystal or model of the present invention can be screened in assays such as those well known in the art. Screening can be, for example, in vitro, in cell culture, and/or in vivo. Biological screening assays preferably centre on activity-based response models, binding assays (which measure how well a compound binds to the receptor), and bacterial, yeast and animal cell lines (which measure the biological effect of a compound in a cell). The assays can be automated for high capacity-high throughput screening (HTS) in which large numbers of compounds can be tested to identify compounds with the desired activity. The biological assay, may also be an assay for the ligand binding activity of a compound that selectively binds to the LBD compared to other nuclear receptors.

Ligands/Compounds/Modulators

The present invention provides a ligand or compound or entity identified by a screening method of the present invention. A ligand or compound may have been designed rationally by using a model according to the present invention. A ligand or compound identified using the screening methods of the invention specifically associate with a target compound. In the present invention the target compound may be the mannosidase II enzyme or a molecule that is capable of associating with the mannosidase II enzyme (for example a substrate molecule). In a preferred embodiment the ligand is capable of binding to the LBD of a mannosidase II.

A ligand or compound identified using a screening method of the invention may act as a “modulator”, i.e. a compound which affects the activity of a mannosidase II. A modulator may reduce, enhance or alter the biological function of a mannosidase II. For example a modulator may modulate the capacity of the enzyme to hydrolyse mannose residues. An alteration in biological function may be characterised by a change in specificity. For example, a modulator may cause the enzyme to accept a different substrate molecule to transfer a different sugar, or to work with a different metal cofactor In order to exert its function the modulator commonly binds to the ligand binding domain

A “modulator” which is capable of reducing the biological function of the enzyme may also be known as an inhibitor. Preferably an inhibitor reduces or blocks the capacity of the enzyme to hydrolyse mannose residues. The inhibitor may mimic the binding of a substrate molecule, for example, it may be a substrate analogue. A substrate analogue may be designed by considering the interactions between the substrate molecule and the enzyme (for example by using information derivable from the crystal of the invention) and specifically altering one or more groups (as described above).

In a highly preferred embodiment a modulator acts as an inhibitor of the mannosidase II and is capable of inhibiting N-glycan biosynthesis. In another embodiment, a modulator enhances mannosidase II activity and is capable of regulating the immune system.

The present invention also provides a method for modulating the activity of a mannosidase II within a cell using a modulator according to the present invention. It would be possible to monitor the expression of N-glycans on the cell surface following such treatment by a number of methods known in the art (for example by detecting expression with an N-glycan specific antibody).

In another preferred embodiment, the modulator modulates the catalytic mechanism of the enzyme.

A modulator may be an agonist, partial agonist, partial inverse agonist or antagonist of the mannosidase II.

As used herein, the term “agonist” means any ligand, which is capable of binding to a ligand binding domain and which is capable of increasing a proportion of the enzyme that is in an active form, resulting in an increased biological response. The term includes partial agonists and inverse agonists.

As used herein, the term “partial agonist” means an agonist that is unable to evoke the maximal response of a biological system, even at a concentration sufficient to saturate the specific receptors.

As used herein, the term “partial inverse agonist” is an inverse agonist that evokes a submaximal response to a biological system, even at a concentration sufficient to saturate the specific receptors. At high concentrations, it will diminish the actions of a full inverse agonist.

The invention relates to a mannosidase II ligand binding domain antagonist, wherein said ligand binding domain is that defined by the amino acid structural coordinates described herein. For example the ligand may antagonise the inhibition of mannosidase by swainsonine.

As used herein, the term “antagonist” means any agent that reduces the action of another agent, such as an agonist. The antagonist may act at the same site as the agonist (competitive antagonism). The antagonistic action may result from a combination of the substance being antagonised (chemical antagonism) or the production of an opposite effect through a different receptor (functional antagonism or physiological antagonism) or as a consequence of competition for the binding site of an intermediate that links receptor activation to the effect observed (indirect antagonism).

As used herein, the term “competitive antagonism” refers to the competition between an agonist and an antagonist for a receptor that occurs when the binding of agonist and antagonist becomes mutually exclusive. This may be because the agonist and antagonist compete for the same binding site or combine with adjacent but overlapping sites. A third possibility is that different sites are involved but that they influence the receptor macromolecules in such a way that agonist and antagonist molecules cannot be bound at the same time. If the agonist and antagonist form only short lived combinations with the receptor so that equilibrium between agonist, antagonist and receptor is reached during the presence of the agonist, the antagonism will be surmountable over a wide range of concentrations. In contrast, some antagonists, when in close enough proximity to their binding site, may form a stable covalent bond with it and the antagonism becomes insurmountable when no spare receptors remain.

As mentioned above, an identified ligand or compound may act as a ligand model (for example, a template) for the development of other compounds. A modulator may be a mimetic of a ligand or ligand binding domain. A mimetic of a ligand may compete with a natural ligand for a mannosidase II and antogonize a physiological effect of the enzyme in an animal. A mimetic of a ligand may be an organically synthesized compound. A mimetic of a ligand binding domain, may be either a peptide or other biopharmaceutical (such as an organically synthesized compound) that specifically binds to a natural substrate molecule for a mannosidase II and antagonize a physiological effect of the enzyme in an animal.

A modulator may be one or a variety of different sorts of molecule. For example, a modulator may be a peptide, member of random peptide libraries and combinatorial chemistry-derived molecular libraries, phosphopeptide (including members of random or partially degenerate, directed phosphopeptide libraries), a carbohydrate, a monosaccharide, an oligosaccharide or polysaccharide, a glycolipid, a glycopeptide, a saponin, a heterocyclic compound antibody, carbohydrate, nucleoside or nucleotide or part thereof, and small organic or inorganic molecule. A modulator may be an endogenous physiological compound, or it may be a natural or synthetic compound. The modulators of the present invention may be natural or synthetic. The term “modulator” also refers to a chemically modified ligand or compound, and includes isomers and racemic forms.

Once a ligand has been optimally selected or designed, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to the mannosidase II LBD by the same computer methods described above.

Preferably, positions for substitution are selected based on the predicted binding orientation of a ligand to the mannosidase II LBD.

A technique suitable for preparing a modulator will depend on its chemical nature. For example, organic compounds may be prepared by organic synthetic methods described in references such as March, 1994, Advanced Organic Chemistry: Reactions, Mechanisms, and Structure, New York, McGraw Hill. Peptides can be synthesized by solid phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Once cleaved from the resin, the peptide may be purified by preparative high performance liquid chromatography (e.g., Creighton (1983) Proteins Structures and Molecular Principles, W H Freeman and Co, New York N.Y.). The composition of the synthetic peptides may be confirmed by amino acid analysis or sequencing (e.g., the Edman degradation procedure; Creighton, supra).

If a modulator is a nucleotide, or a polypeptide expressable therefrom, it may be synthesized, in whole or in part, using chemical methods well known in the art (see Caruthers M H et al (1980) Nuc Acids Res Symp Ser 215-23, Horn T et al (1980) Nuc Acids Res Symp Ser 225-232), or it may be prepared using recombinant techniques well known in the art.

Direct synthesis of a ligand or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences obtainable from the ligand, or any part thereof, may be altered during direct synthesis and/or combined using chemical methods with a sequence from other subunits, or any part thereof, to produce a variant ligand.

In an alternative embodiment of the invention, the coding sequence of a ligand or mimetics thereof may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

A wide variety of host cells can be employed for expression of the nucleotide sequences encoding a ligand of the present invention. These cells may be both prokaryotic and eukaryotic host cells. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the expression products to produce an appropriate mature polypeptide. Processing includes but is not limited to glycosylation, ubiquitination, disulfide bond formation and general post-translational modification.

In an embodiment of the present invention, the ligand may be a derivative of, or a chemically modified ligand. The term “derivative” or “derivatised” as used herein includes the chemical modification of a ligand.

A chemical modification of a ligand and/or a key amino acid residue of a ligand binding domain of the present invention may either enhance or reduce hydrogen bonding interaction, charge interaction, hydrophobic interaction, Van Der Waals interaction or dipole interaction between the ligand and the key amino acid residue(s) of the mannosidase II LBD. By way of example, steric hinderance is a common means of changing the interaction of the mannosidase II LBD binding domain with the activation domain.

Preferably such modifications involve the addition of substituents onto a test compound such that the substituents are positioned to collide or to bind preferentially with one or more amino acid residues that correspond to the key amino acid residues of mannosidase II LBD of the present invention. Typical modifications may include, for example, the replacement of a hydrogen by a halo group, an alkyl group, an acyl group or an amino group.

The invention also relates to classes of modulators of mannosidase II based on the structure and shape of a substrate, defined in relation to the substrate's molecule's spatial association with a mannosidase II structure of the invention or part thereof. Therefore, a modulator may comprise a substrate molecule having the shape or structure, preferably the structural coordinates, of a substrate molecule in the active site binding pocket of a reaction catalyzed by a mannosidase II. In an embodiment, the substrate comprises GlcNAcMan₅GlcNAc₂-Asn-.

A modulator may be an inhibitor of a mannosidase II such as swainsonine or a derivative or mimetic thereof.

A class of modulators of mannosidase 11 enzymes may comprise a compound containing a structure of swainsonine, and having one or more, preferably all, of the structural coordinates of swainsonine of Table 2 or 8. Functional groups in the swainsonine modulators may be substituted with, for example, alkyl, alkoxy, hydroxyl, aryl, cycloalkyl, alkenyl, alkynyl, thiol, thioalkyl, thioaryl, amino, or halo, or they may be modified using techniques known in the art. Substituents will be selected to optimize the activity of the modulator.

### Pharmaceutical Composition

The present invention also provides the use of a ligand or modulator according to the invention, in the manufacture of a medicament to treat and/or prevent a disease in a mammalian patient. There is also provided a pharmaceutical composition comprising such a ligand or modulator and a method of treating and/or preventing a disease comprising the step of administering such a modulator or pharmaceutical composition to a mammalian patient.

In an embodiment, the invention relates to a pharmaceutical composition which comprises a crystal structure of the invention or a part thereof (e.g. a binding domain), or a modulator of the invention in an amount effective to regulate one or more of the conditions described herein (e.g. tumor growth or metastasis) and a pharmaceutically acceptable carrier, diluent or excipient.

The pharmaceutical compositions may be for human or animal usage in human and veterinary medicine and will typically comprise a pharmaceutically acceptable carrier, diluent, excipient, adjuvant or combination thereof.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

A pharmaceutical composition of the invention can be administered to a subject in an appropriate carrier or diluent, co-administered with enzyme inhibitors or in an appropriate carrier such as microporous or solid beads or liposomes. Liposomes include water-in-oil-in-water emulsions as well as conventional liposomes (Strejan et al., (1984) J. Neuroimmunol 7:27).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may also be used.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the pharmaceutical composition is to be delivered mucosally through the gastrointestinal mucosa, it should be able to remain stable during transit through the gastrointestinal tract; for example, it should be resistant to proteolytic degradation, stable at acid pH and resistant to the detergent effects of bile.

It is to be understood that not all of the agent need be administered by the same route.

Where appropriate, the pharmaceutical compositions can be administered by inhalation, in the form of a suppository or pessary, topically in the form of a lotion, gel, hydrogel, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

If the agent of the present invention is administered parenterally, then examples of such administration include one or more of: intravenously, intra-arterially, intraperitoneally, intrathecally, intraventricularly, intraurethrally, intrastemally, intracranially, intramuscularly or subcutaneously administering the agent; and/or by using infusion techniques.

For buccal or sublingual administration the compositions may be administered in the form of tablets or lozenges which can be formulated in a conventional manner.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

As indicated, a therapeutic agent of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Therapeutic administration of polypeptide modulators may also be accomplished using gene therapy. A nucleic acid including a promoter operatively linked to a heterologous polypeptide may be used to produce high-level expression of the polypeptide in cells transfected with the nucleic acid. DNA or isolated nucleic acids may be introduced into cells of a subject by conventional nucleic acid delivery systems. Suitable delivery systems include liposomes, naked DNA, and receptor-mediated delivery systems, and viral vectors such as retroviruses, herpes viruses, and adenoviruses. Applications

The modulators and compositions of the invention may be used to modulate the biological activity of a mannosidase II in a cell, including modulating a pathway in a cell regulated by the mannosidase II or modulating a mannosidase II with inappropriate activity in a cellular organism. In addition, a mannosidase II structure of the invention may be used to devise protocols to modulate the biological activity of a mannosidase II in a cell.

Cellular assays, as well as animal model assays in vivo, may be used to test the activity of a potential modulator of a mannosidase II as well as diagnose a disease associated with inappropriate mannosidase II activity. In vivo assays are also useful for testing the bioactivity of a potential modulator designed by the methods of the invention.

The invention further provides a method of treating a mammal, the method comprising administering to a mammal a modulator or pharmaceutical composition of the present invention.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. By way of example, the pharmaceutical composition of the present invention may be administered in accordance with a regimen of 1 to 10 times per day, such as once or twice per day.

For oral and parenteral administration to human patients, the daily dosage level of the agent may be in single or divided doses.

The modulators (e.g. inhibitors) identified using the methods of the invention may be useful in the treatment and prophylaxis of tumor growth and metastasis of tumors. Anti-metastatic effects of inhibitors can be demonstrated using a lung colonization assay. For example, melanoma cells treated with an inhibitor may be injected into mice and the ability of the melanoma cells to colonize the lungs of the mice may be examined by counting tumor nodules on the lungs after death. Suppression of tumor growth in mice by the inhibitor administered orally or intravenously may be examined by measuring tumor volume.

An inhibitor identified using the invention may have particular application in the prevention of tumor recurrence after surgery i.e. as an adjuvant therapy.

An inhibitor may be especially useful in the treatment of various forms of neoplasia such as leukemias, lymphomas, melanomas, adenomas, sarcomas, and carcinomas of solid tissues in patients. In particular, inhibitors can be used for treating malignant melanoma, pancreatic cancer, cervico-uterine cancer, ovarian cancer, cancer of the kidney such as metastatic renal cell carcinoma, stomach, lung, rectum, breast, bowel, gastric, liver, thyroid, head and neck cancers such as unresectable head and neck cancers, lymphangitis carcinamatosis, cancers of the cervix, breast, salivary gland, leg, tongue, lip, bile duct, pelvis, mediastinum, urethra, bronchogenic, bladder, esophagus and colon, non-small cell lung cancer, and Karposi's Sarcoma which is a form of cancer associated with HIV-infected patients with Acquired Immune Deficiency Syndrome (AIDS). The inhibitors may also be used for other anti-proliferative conditions such as bacterial and viral infections, in particular AIDS.

An inhibitor identified in accordance with the present invention may be used to treat immunocompromised subjects. For example, they may be used in a subject infected with HIV, or other viruses or infectious agents including bacteria, fungi, and parasites, in a subject undergoing bone marrow transplants, and in subjects with chemical or tumor-induced immune suppression.

Inhibitors may be used as hemorestorative agents and in particular to stimulate bone marrow cell proliferation, in particular following chemotherapy or radiotherapy. The myeloproliferative activity of an inhibitor of the invention may be determined by injecting the inhibitor into mice, sacrificing the mice, removing bone marrow cells and measuring the ability of the inhibitor to stimulate bone marrow proliferation by directly counting bone marrow cells and by measuring clonogenic progenitor cells in methylcellulose assays. The inhibitors can also be used as chemoprotectants, and in particular to protect mucosal epithelium following chemotherapy.

An inhibitor identified in accordance with the invention also may be used as an antiviral agent in particular on membrane enveloped viruses such as retroviruses, influenza viruses, cytomegaloviruses and herpes viruses. An inhibitor may also be used to treat bacterial, fungal, and parasitic infections. An inhibitor may also be used in the treatment of inflammatory diseases such as rheumatoid arthritis, asthma, inflammatory bowel disease, and atherosclerosis.

An inhibitor may also be used to augment the anti-cancer effects of agents such as interleukin-2 and poly-IC, to augment natural killer and macrophage tumoricidal activity, induce cytokine synthesis and secretion, enhance expression of LAK and HLA class I specific antigens; activate protein kinase C, stimulate bone marrow cell proliferation including hematopoietic progenitor cell proliferation, and increase engraftment efficiency and colony-forming unit activity, to confer protection against chemotherapy and radiation therapy (e.g. chemoprotective and radioprotective agents), and to accelerate recovery of bone marrow cellularity particularly when used in combination with chemical agents commonly used in the treatment of human diseases including cancer and acquired immune deficiency syndrome (AIDS). For example, an inhibitor can be used as a chemoprotectant in combination with anti-cancer agents including doxorubicin, 5-fluorouracil, cyclophosphamide, and methotrexate, and in combination with isoniazid or NSAID.

Alpha-mannosidosis may also be amendable to treatment or prophylaxis by the method of the present invention.

The loss of mannosidase II has been found to alter N-glycan branching and attenuate the immune system's ability to maintain self-tolerance (Chui et al, PNAS 98(3):1142-1147, 2001). Therefore, the structures, modulators, compositions, and methods of the invention may be useful in the treatment or prophylaxis of autoimmune disease including systemic lupus erythematosus.

The present invention thus provides a method for treating the above-mentioned conditions in a subject comprising administering to a subject an effective amount of a modulator of the invention. The invention also contemplates a method for stimulating or inhibiting tumor growth or metastasis in a subject comprising administering to a subject an effective amount of a modulator of the invention.

The following non-limiting examples are illustrative of the present invention.

### EXAMPLES

### Example 1

### Drosophila Mannosidase II Preparation and Structure Determination

Expression Plasmids

Constructs designed to expressed dGMII in Drosophila Schneider (S2) cells were based on the DES expression system available from InVitrogen with extensive modifications. Expression plasmids were constructed which had the dGMII under the control either of the inducible metallothioneine (MT) promoter or the strong constitutive actin 5.1 promoter (AC5). Amino terminal purification tags were inserted in place of the C-terminal tags in the commercially available vectors. Earlier attempts, to truncate the mouse enzyme from at the C-terminus resulted in inactive protein, as had also been noted with the GlcNAc-transferases. Thus, it was elected to keep the C-terminus free. Expression vectors were created with either a 6His-tag, for purification on metal chelate columns such as Ni-NTA (Qiagen) or cobalt based Talon columns (Clontech), or with a Strep-tag for purification on streptavidin-Sepharose. These affinity tags are initially non-cleavable and add approximately 8-10 residues to the end of the protein. Finally, constructs were made either lacking or containing the Bip secretion sequence to direct the expressed protein into the cells or medium respectively.

Blasticidin Selection

Initial attempts at stable transfection with the recommended hygromycin selection system were unsuccessful. Therefore a new selection plasmid, pCopBlast was created which encodes blasticidin S deaminase under the control of the constitutive copia promoter. Blasticidin S has been used for stable transfectants of mammalian and plant cells, as well as yeast. Commercially available control plasmids expressing MT-induced secreted green fluorescent protein (GFP), or constitutive and MT-induced unsecreted bacterial β-galactosidase (LacZ) were used to test the suitability of blasticidin selection in S2 cells, and to optimize conditions for transfection, selection, and metallothionein induction. Stable transfectants could be selected with 16 μg/ml blasticidin in Schneider's S2 medium containing 10% fetal bovine serum. Copper and cadmium were the only metals found to activate the MT promoter; copper favoured internally expressed proteins and cadmium, secreted proteins. Maintenance of the altered phenotype was also demonstrated for many weeks in the absence of the selective pressure of blasticidin demonstrating that these were indeed stably transfected cell lines.

Creation of Stably Expressing dGMII Cell Lines.

Starting with the pProtA expression plasmid from initial published studies [Rabouille et al, 1999], the mannosidase coding region was excised, and inserted into an in-frame EcoRI site immediately at the end of the affinity tag in the new plasmids. The position of a unique 3′ restriction site outside the coding region meant that 100-200 bp of extra sequence was added between the stop codon and the SV40 polyadenylation site. This extra sequence was removed with a short PCR amplification using a unique internal restriction site. Both ends of the constructs were sequenced to verify proper reading frame and lack of PCR errors. The resulting constructs consist of the dGMII catalytic region with a short length of the stalk region, in a variety of “flavours” of promoter, affinity tag, and expression location.

Co-transfection of the pCopBlast selection plasmid with the mannosidase expression plasmids, followed by selection for blasticidin resistance allowed stable expressing cell lines after approximately one month. Mannosidase activity was measured using PNP-mannoside, in a microtitre plate assay. Protein was detected on Western blots using anti-PentaHis antibody (Qiagen). Only the secreted products showed activity, with similar levels in the constitutive and MT-promoter constructs. No difference in mannosidase activity was seen between His or Strep tagged protein. All subsequent work was carried out with the secreted constructs.

Insect cells do not grow at low population densities. Therefore, the initial population of selected cells was a mixed population with each cell in the culture having somewhat different levels of incorporated expression plasmid. To select individual cells with high levels of expression the stably transfected population was diluted to single cells in a 50:50 mix of conditioned medium and fresh medium with blasticidin. These were then plated in 96-well culture plates. After five weeks, about 10% of the wells showed growths of colonies large enough to transfer, of which roughly 30% had activity. The highest expressors had approximately 5 times the activity of the initial population in the MT-inducible strains. High-expressing clones of the constitutively expressed dGMII, were obtained suggesting that the continued production mannosidase by the cells may be detrimental, especially under the stressful conditions of single-cell selection.

Expression and Purification of dGMII.

The availability of a stable clones expressing considerable amounts of mannosidase allowed optimization of induction, expression and purification conditions. In contrast to mammalian cells, insect cells are not highly adherent and will grow to high cell densities in a variety of culture vessels including roller bottles, spinners, fermentors and shake flasks. No CO₂ is required, and temperatures in the range of 25-28° C. are optimal. With stably transfected cells, the difficulties that accompany baculoviral infection do not arise.

Initial experiments were carried out in S2 medium containing 10% bovine serum. Metal concentrations used to induce and time of induction were optimized for dGMII production. 10-20 μM cadmium proved optimal for induction. Although copper (at approximately 500-1000 μM) is generally used in the literature for induction, the sensitivity of dGMII to inhibition by copper (IC₅₀=25 μM,[26]) precluded its use. Cadmium has been reported to be detrimental to the growth of cells. However, at the concentrations used here, the cells continued to grow and maintain greater than 90% viability (as assessed by Trypan blue exclusion) until the end of the induction period. Cells were maintained in the continous presence of cadmium for up to three passages.

As the dGMII was secreted into the medium, it was badly contaminated with bovine serum albumin (BSA). Attempts to remove the impurity by Blue Agarose or Ni-NTA chromatography were unsuccessful. To circumvent this contamination problem a number of serum-free media were evaluated for growth and expression levels. There are very few serum-free media developed for Drosophila cells so ones that have been used with baculovirus expression systems were evaluated. Ultimately the Excel420 medium from JRH Biosciences was successful.

A further advantage to this medium is the incorporation of seleno-methionine in place of methionine for crystallographic phasing purposes. A custom preparation of this medium was purchased from JRH free of Met and Cu. Inclusion of 50 μg/ml of SeMet resulted in the production of protein with high enough incorporation (approximately 50% by mass spectrometry) for accurate phasing.

Cells were adapted to serum-free growth by gradual dilution with CCM3 medium and then they were switched into the other media for the expression studies. Excel420, CCM3 and SFX-Insect were clearly superior for maintaining healthy growth, though CCM3 provided slightly lower levels of expression. Levels of cadmium required for induction were optimized for each medium and were considerably lower than those required in S2 medium. For unknown reasons, constitutive expression of dGMII was much lower in serum-free medium. Therefore, all subsequent scale-up and purifications were carried out with the MT-inducible 6His tagged constructs.

To scale-up protein expression cells were first grown as suspensions in spinner cultures. These were subsequently put into 2.8 litre Fernbach flasks (1 litre Excel 420/flask) shaken at 100 rpm at 28° C. Cells were induced for 72 hours with 10 μM cadmium. After this time the medium was asceptically harvested and the cells are placed in the same volume of fresh medium for a further round of induction. This can be repeated at least one more time without significant cell death or loss of protein expression. Based on activity measurements up to 50 mg/litre of medium can be expressed every three days. This is approximately 1000 fold greater than in initial expression experiments in CHOP cells [Rabouille et al, 1999]. This procedure requires about 2 weeks of dedicated time in an incubator/shaker.

Purification is effected by batch binding first to Blue-Agarose, with elution by 350 mM NaCl, and then to Ni-NTA resin, with elution by 50 mM imidizole. Initial, secreted protein from the medium of the serum-free grown cells was loaded in batch to Blue-Agarose. The beads were then loaded into a column and washed with 20 column volumes of 50 mM NaCl in 20 mM Tris pH8. The majority of the mannosidase was eluted with 350 mM NaCl. This pooled eluant was loaded onto NiNTA, washed with low imidizole, and eluted with 50 mM imidizole to achieve crystallization purity. The protein is then dialysed extensively against 10 mM Tris, pH 8.3 and 100 mM NaCl and concentrated (to greater than 20 mg/ml) for crystallization trials. All crystallization has been carried out from a single protein preparation.

Crystallization

Crystals of Drosophila Mannosidase II and complexes of the enzyme with various inhibitors were grown at room temperature using vapor diffusion and micro-batch crystallization techniques. Crystals were obtained under a wide variety of conditions. Polyethylene glycol (PEG) was used as a precipitant (with sizes: 4000; 6000; 8000; 10000; and 20000) at concentrations varying from 5-20%, in the presence of 5% 2,4-methyl-pentanediol (MPD) or 0-30% glycerol. Crystallization solutions were buffered at pH 7-7.5 using 100 mM buffer solutions of Tris, Hepes or Mes. The crystals belong to the orthorhombic space group P2₁2₁2₁ with cell dimensions: a=69 Å; b=110 Å; c=139 Å; α=90°; β=90°; γ=90°. For the initial structure determination Seleno-Methionine-derivatized Mannosidase II crystals were grown in 8.5% PEG 6000, 5% MPD and 100 mM Tris pH 7.0, using micro seeds obtained from wild-type enzyme crystals. Data were collected from crystals that were frozen in liquid nitrogen after a stepwise increase of the MPD concentration in the crystallization solution from 5% to 25%.

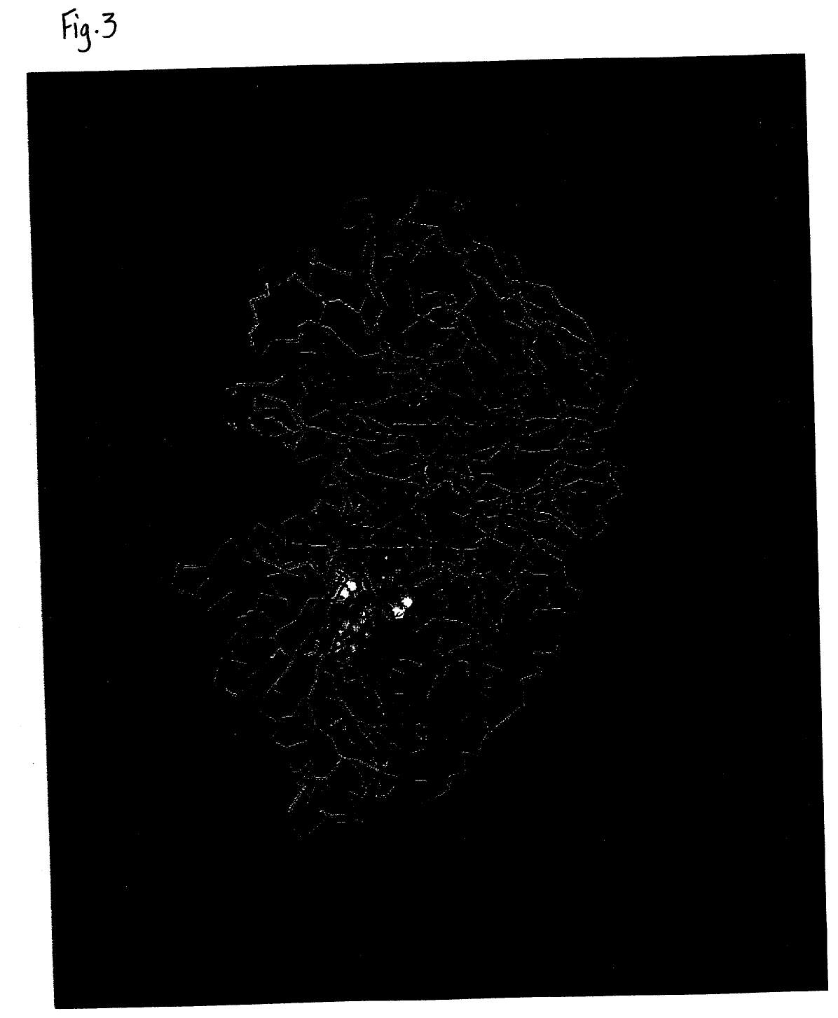

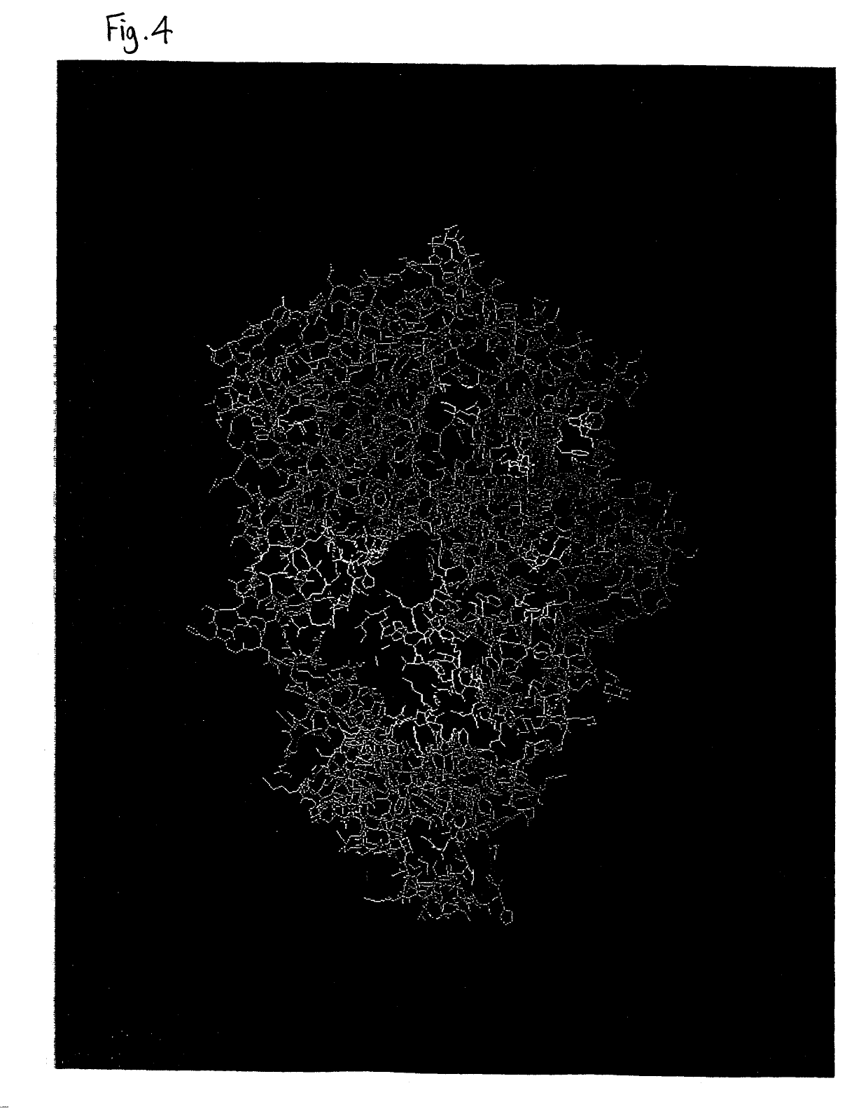

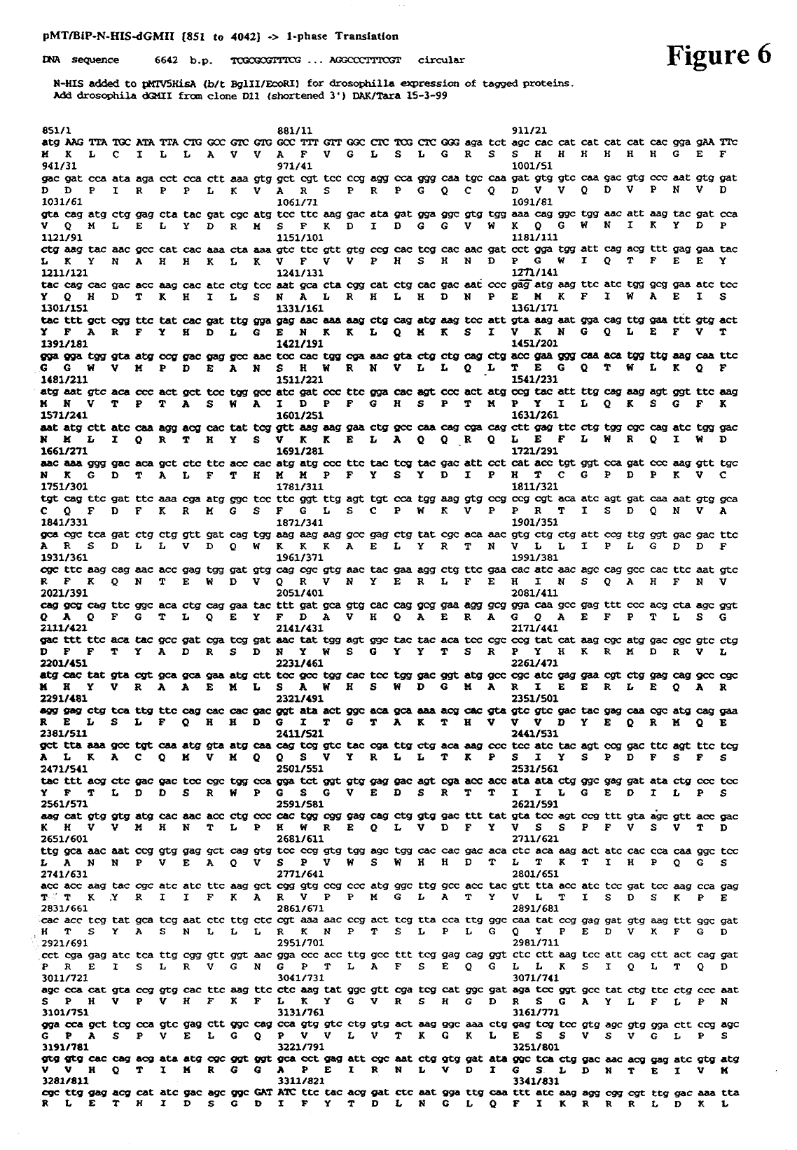

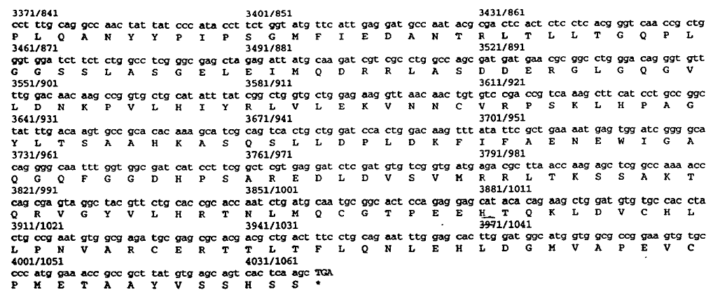

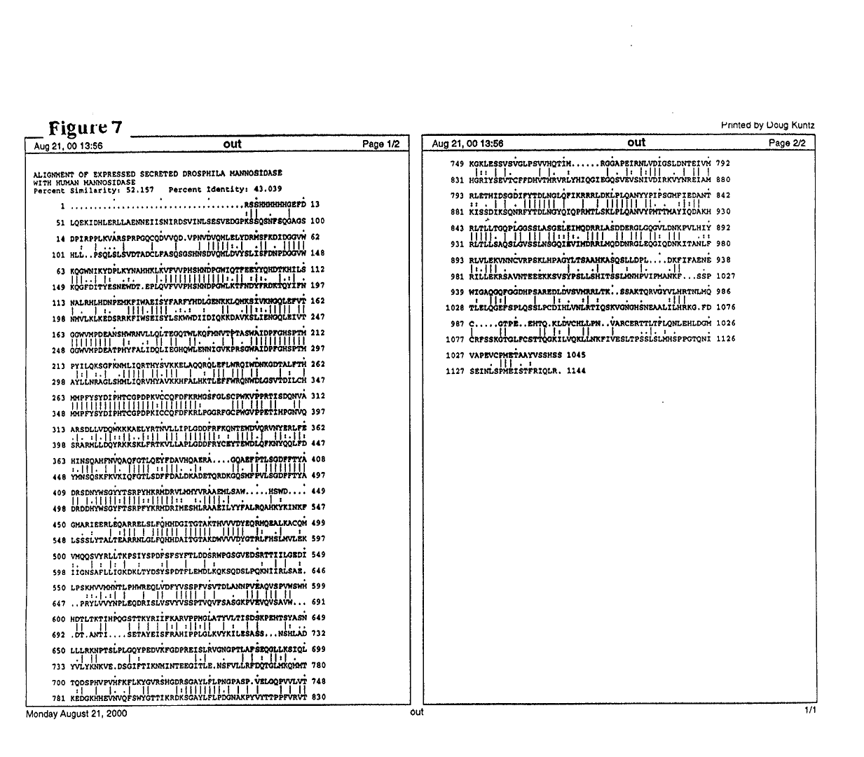

A crystal of the invention is illustrated in the Figures. In particular, FIG. 1 shows the active site of a mannosidase II. FIG. 2 shows the secondary structure of Drosophila Golgi α-mannosidase II. Helices are in blue and β sheets are in red. FIG. 3 shows the Drosophila golgi α-mannosidase II molecule with the colours representing where it is identical to human GMII. The red and blue represent deletions or insertions with respect to the human sequence. The green is a disulphide bond. FIG. 4 shows the whole Drosophila golgi α-mannosidase II molecule in sticks with residues that are identical in the lysosomal manII as coloured balls (red or blue depending whether they are in the N-terminal or C-terminal part of the molecule). FIG. 5 shows the active site of a Drospholiga mannosidase. FIG. 6 shows the DNA sequence of an expressed Drosophila mannosidase. FIG. 7 shows an alignment of expressed secreted Drosophila mannosidase with human mannosidase.

### Example 2

### Experimental Procedures

Protein Overexpression and Purification

Expression, purification and crystallization of the dGMII will be described in detail elsewhere. Briefly, the cDNA was inserted behind an inducible promoter, and used to stably transfect Drosophila S2 cells. Single cell clones secreting high levels of dGMII were chosen and adapted to serum-free medium. Unlabelled dGMII was isolated from the supernatants of cells grown in Fernbach flasks by batch binding to Blue-Agarose (Sigma). The protein was eluted from the Blue-Agarose using NaCl and further purified by Ni-NTA chromatography (Qiagen). EDTA (5 mM) was added to scavenge any free nickel. The protein was extensively dialyzed against 10 mM Tris pH 8 containing 100 mM NaCl, concentrated to 25 mg/ml, and stored in aliquots at −80° C.

For seleno-methionine labeling, a custom batch of Ex-Cell 420 (#006140E JRH Biosciences, Lenexa K S) was used which lacked any added methionine or copper. Cells were grown to high cell density in a spinner flask in standard medium, resuspended in the “methionine-free” medium and allowed to starve for 4 hours prior to the addition of 50 mg/l of seleno-methionine (Sigma). After 70 hrs of induction the protein was purified from the supernatant as outlined above except that 5 mM β-mercaptoethanol was present throughout the purification.

Crystallization and Data Collection

Crystals of Drosophila Mannosidase II and complexes of the enzyme with various inhibitors were grown at room temperature using vapor diffusion and micro-batch crystallization techniques. Crystals were obtained under a wide variety of conditions. Polyethylene glycol (PEG) was used as a precipitant (with sizes: 4000; 6000; 8000; 10000; and 20000) at concentrations varying from 5-20%, in the presence of 5% 2,4-methyl-pentane-diol (MPD) or 0-30% glycerol. Crystallization solutions were buffered at pH 7-7.5 using 100 mM buffer solutions of Tris, Hepes or Mes. The crystals belong to the orthorhombic space group P2₁2₁2₁ with cell dimensions: a=69 Å; b=110 Å; c=139 Å; α=90°; β=90°; γ=90°. For the initial structure determination Seleno-Methionine-derivatized Mannosidase II crystals were grown in 8.5% PEG 6000, 5% MPD and 100 mM Tris pH 7.0, using micro seeds obtained from wild-type enzyme crystals. Data were collected from crystals that were frozen in liquid nitrogen after a stepwise increase of the MPD or glycerol concentration in the crystallization solution from 5% to 25%. Data collection was performed at the Advanced Photon Source facility at Argonne National Laboratories, Argonne, Ill. Beam line BM14D was used for collection of multiple wavelength anomalous dispersion data and BM14C for collection of high-resolution data.

Structure Determination

The structure of uncomplexed dGMII was determined by MAD phasing at the Selenium absorption edge with datasets collected at an absorption peak wavelength of 0.9786 Å, inflection wavelength of 0.9790 Å and a remote wavelength of 0.9770 Å. Initial positions of 26 out of 28 Selenium atoms were determined with the program Solve (Terwilliger et al., 1987) with an initial Figure of Merit (FOM) of 0.67. The experimental map obtained after density modification, using the program DM of the CCP4 program package (Cowtan, 1994), showed continuous density of very high quality for the whole molecule. The structure was traced using the program O (Jones et al., 1991) using the density modified experimental map. The model was refined using the program CNS (Brünger et al., 1998).

Inductively Coupled Plasma Atomic Emission Spectroscopy (ICP-AES)

The metal content in dGMII samples was analyzed by inductively coupled plasma atomic emission spectroscopy using the ICP-AES model ‘Optima 3000 DV’ (Dual View) from Perkin Elmer. The zinc content in the protein samples was determined relative to an equivalent amount of dGMII assay buffer.

Results and Discussion

Protein Expression

The cDNA for Drosophila GMII is predicted to encode a protein of 1108 amino acids. For protein expression in Drosophila cells the first 75 amino acids consisting of the cytosolic and transmembrane domains and most of the stalk region were eliminated. The remaining cDNA was cloned in-frame behind a secretion signal.

Numbering of our construct starts at the point where the expressed protein is expected to be cleaved, by signal peptidase, from the secretion signal. Three extra amino terminal residues, a 6-histidine tag, and a glycine, glutamine and phenylalanine were added in cloning. The first aspartate (D13) of the construct corresponds to aspartate 76 of the native protein. The first residue seen in the structure (C31) corresponds to C94, and the final residue S1044 to S1107, of the full-length sequence.

Structure Determinations

The structure of Drosophila Golgi α-mannosidase II has been determined by the multi-wavelength anomalous dispersion (MAD) phasing method using a data set collected from a crystal of Seleno-methionine derivatized enzyme (Table 9). This is the first reported structure of a Se-Met substituted enzyme produced in a Drosophila overexpression system. The native dGMII structure has been refined to a resolution of 1.76 Å with some data to 1.4 Å resolution (see refinement statistics presented in Table 10). The model contains residues 31-1044 of the recombinant enzyme (numbered as described above), as well as a zinc ion, an N-glycan residue, a molecule of the cryo-protectant, 2-methyl-2,4-pentanediol (MPD), and a tris(hydroxymethyl)-aminomethane (Tris) molecule. The presence of the enzyme-bound zinc ion was confirmed by Inductively Coupled Plasma Atomic Emission Spectroscopy (ICP-AES). The final structure of the dGMII-swainsonine complex has been refined at 1.87 Å resolution and the dGMII-DMNJ complex to 1.69 Å resolution, with some data to 1.5 Å resolution.

Overall Architecture of dGMII

The structure of dGMII reveals a previously unobserved protein fold consisting of an N-terminal α/β domain, a three-helical bundle and an all-β C-terminal domain forming a single compact entity, connected by 5 internal disulfide bonds and stabilized by a zinc binding site (FIG. 8B). The oval shaped molecule has two distinct faces (FIG. 8C). The N-terminal face of the molecule is convex, whereas the opposing face of the enzyme has a planar surface. N-terminal residue Cys-31 is the last residue of the so-called stalk region, the linkage between the catalytic domain and the transmembrane domain. Cys-31 is located at the convex face of the molecule, indicating that this surface of the molecule presumably faces the inner side of the Golgi membrane, while the planar surface, containing the active site cavity (see below), faces the Golgi lumen.

The N-terminal α/β domain is comprised of an inner core of three β-sheets (A, B and C, FIG. 8B) consisting of 11, mostly parallel β-strands, surrounded by 16 α-helices. This domain contains a GlcNAc residue found in the electron density map at a consensus N-glycosylation site (Asn-194), located at the N-terminus of helix 7. The α/β domain is stabilized by three disulfide bonds: between Cys-31 and Cys-1032 connecting the N and C-terminal extremes of dGMII; Cys-275 and Cys-282 linking helices 10 and 11; Cys-283 and Cys-297 linking helix 11 with a loop between helix 13 and the core of parallel β-sheets. The cysteines forming the latter two disulfide-bonds are conserved in the human Golgi α-mannosidase II sequence.

The C-terminal half of the protein contains a three-helix bundle, comprised of helices 18, 20 and 21, and is connected to the N-terminal α/β-domain via a zinc binding site. The zinc ion is coordinated in a T₅-square-based pyramidal geometry involving residues: Asp-90, His-92, Asp-204 and His-471. Furthermore, the C-terminal domain contains two immunoglobulin-like domains: a small β-sandwich consisting of 12 anti-parallel strands from β-sheets D and E, and a large 21 -strand structure involving β-sheets F and G.

A barrel formed by the three-helix bundle and helix-23 together with the two β-sandwich structures result in a narrow pore in the center of the C-terminal domain. The pore is lined by six arginine residues: Arg-540, 565, 617, 770, 777 and 893, contributing to the overall positive charge of the pore (FIG. 9A). A hairpin loop, connecting two strands of β-sheet D (FIGS. 8B and C, residues 527-540, shown in yellow) protrudes into the center of the barrel on the planar side of the molecule. Arginine residue 530, located at the tip of the type-I β-turn in this loop, plugs the pore preventing an open channel through the protein. The resulting crater-like cavity on the convex side of the molecule is 20 Å deep, with a diameter of 20 Å funneling to 8 Å at the bottom of the cavity. B-factor values of residues within the loop indicate a higher degree of flexibility compared to the rest of the structure (average B-factor values: ˜33 Å² and ˜15 Å², respectively).

Active Site

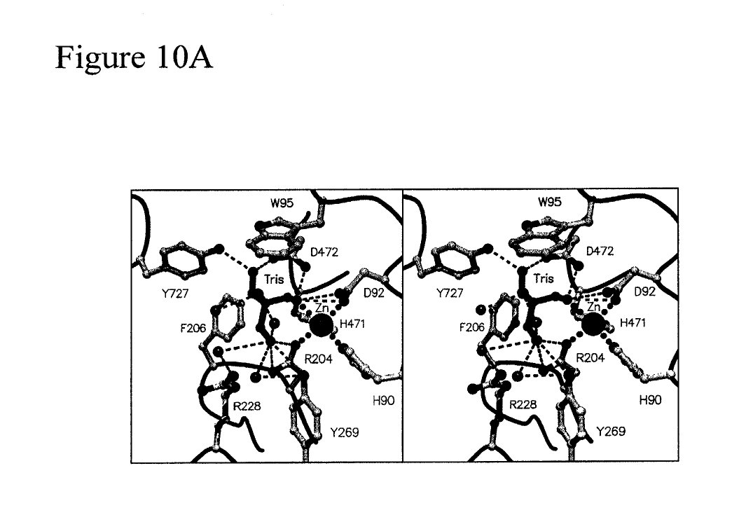

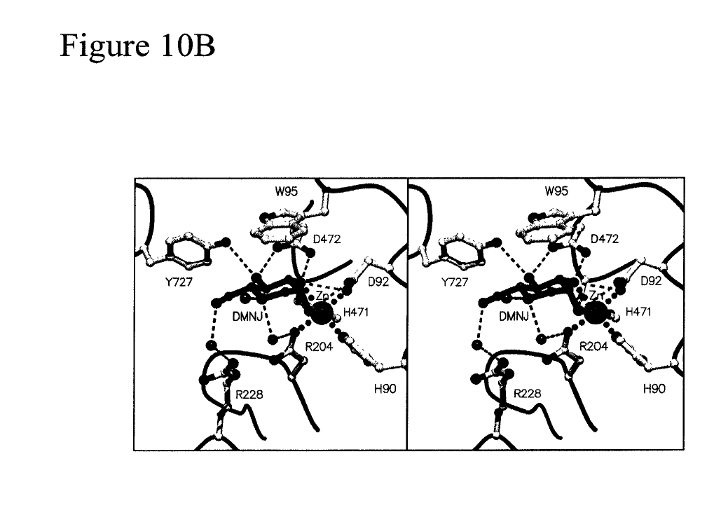

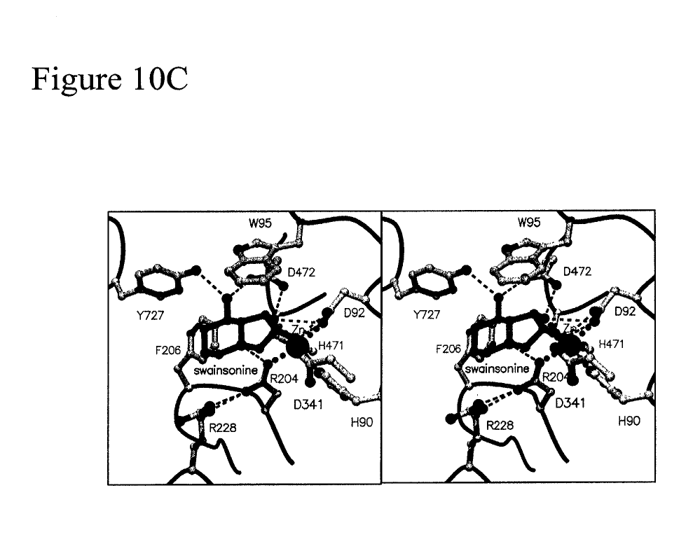

The molecular surface representation of the planar face of dGMII reveals an extended pocket in the N-terminal α/β-domain, formed primarily by acidic residues (FIG. 9B). These same residues form the core of a large, contiguous, surface-exposed patch, of highly conserved amino acids, in comparison with the human GMII sequence (FIG. 9C). The active site of the enzyme is located in a small cavity in the side of this conserved, negatively charged region. The cavity is lined by aromatic residues Trp-95, Phe-206, Tyr-269 and Tyr-727, which are involved in hydrophobic and hydrogen-bond interactions with a bound Tris molecule in the unliganded structure (FIG. 10A). Tris is known to inhibit dGMII activity (Rabouille et al., 1999). Additional hydrophobic and hydrogen bond interactions are observed with Asp-92 and Asp-204. At the open side of the cavity the Tris molecule hydrogen bonds with Arg-228, Tyr-269 and Asp-341 (not shown) via water molecules.

A key feature of the active site is the coordination of the zinc ion by the Tris hydroxyl group O2. In the enzyme-Tris complex the zinc ion is bound in a T₅-square-based pyramidal geometry, coordinated by the ODI oxygen moieties of aspartate residues 92 and 204; the NE2 nitrogens of histidines 90 and 471; and the hydroxyl oxygen O2 of the bound Tris molecule, as represented in FIG. 10A. The T₅ geometry is further stabilized by hydrogen bonds between the zinc coordinating atoms and the existence of H-bonds between the ND 1 nitrogen atoms of the histidines 90 and 471 with the carbonyl oxygen of seleno-methionine 167 and a water molecule, respectively (not shown). The presence of these, so called, ‘elec-His-Zn motifs’ is believed to increase the basicity and the ligand strength of the histidine and arrange it correctly for interaction with the metal (Alberts et al., 1998). In an uninhibited enzyme, Tris would likely be replaced by a coordinating water molecule. As discussed below, this arrangement has implications for substrate binding and transition state stabilization.

The occurrence of zinc in Family 38 glycosyl hydrolases has been described by Snaith (1975) in Jack-bean α-mannosidase. A possible role for zinc in catalysis was indicated by inactivation of the enzyme by chelating agents and bivalent metal ions such as Cu²⁺. Copper has also been shown to effectively inactivate Drosophila and mouse GMII (Rabouille et al., 1999).

Inhibitor Binding

The structures of dGMII in complex with the inhibitors DMNJ and swainsonine show that both compounds bind to the same active site in a similar manner (FIGS. 10B and C). The binding of both inhibitors involves a large contribution of hydrophobic interactions involving aromatic residues Trp-95, Phe-206 and Tyr-727, forming the walls of the cavity. The inhibitor ring structures are stacked against Trp-95, a feature seen in several carbohydrate binding and hydrolyzing proteins (see Boraston et al., 2000 and review papers therein), and stabilized by hydrogen bonds and interactions with the zinc ion. In the complexes of dGMII with either DMNJ or swainsonine the T₅ geometry of the bound zinc ion, as seen in the Tris-bound enzyme, is transformed into T₆-octahedral coordination. In both the dGMII complexes the inhibitor O2 hydroxyl oxygen replaces the O2 oxygen of Tris and the O3 hydroxyl oxygen forms the apex of the second pyramid. In order to obey the restraints of the T₆ geometry, the plane of the swainsonine ring structure is tilted with respect to the saccharide-like ring of the bound DMNJ molecule. This enables the formation of a hydrogen bond between the zinc-coordinating OD1 oxygen of Asp-204 and the N4 nitrogen at the fusion of the five and six-membered rings of swainsonine. As in the Tris-bound enzyme, the zinc coordinating oxygen atoms of the inhibitors are involved in hydrogen bond interactions with the neighboring metal binding residues of the enzyme.

The position of the DMNJ and swainsonine molecules is stabilized in the active site by hydrogen bonds between carboxylic oxygens OD 1 and OD2 of residue Asp-472 and hydroxyl oxygens O3 and O4 (O5 in swainsonine) of the inhibitors, analogous to the O1 and O2 interactions seen in the enzyme-Tris complex. As in the Tris-bound enzyme, DMNJ is involved in additional hydrogen bonds, via water molecules, with the NH2 nitrogen of Arg-228, the hydroxyl oxygen of Tyr-269, the backbone carbonyl oxygen of Arg-876 (not shown) and the OD 1 oxygen of Asp-204.

The displacement of the Tris molecule by either of the inhibitors only slightly affects the zinc binding site by weakening the internal hydrogen bonds between Asp-204 and histidines 90 and 471. No major conformational changes are observed between the Tris-bound and the inhibitor-bound mannosidase molecules as their backbones are virtually superimposable, with root-mean-square-deviations between Cα atoms of 0.068 Å (dGMII-DMNJ complex) and 0.087 Å (dGMII-swainsonine complex).

Catalytic Mechanism

Golgi α-mannosidase II is a retaining mannosyl hydrolase, which cleaves the linkage between the C1 atom of M7 and M6 (FIG. 8A) and, respectively, the O3 and O6 atom of the α1,6-linked mannosyl branch (M4) of GlcNAcMan₅GlcNAc₂. The catalytic mechanism is proposed to follow a very similar path to the corresponding retaining α-glycosidases (Braun et al., 1995; White and Rose, 1997). This is a two-stage reaction that usually involves two carboxylic acids, one acting as a nucleophile attacking the glycosidic bond, and the other as a general acid/base catalyst. Nucleophilic attack of one carboxylic acid results in glycosylation of the enzyme by forming a covalent intermediate followed by a second deglycosylation step, each step passing through an oxocarbonium ion-like transition state.

Based on the structure of the dGMII-inhibitor complexes we speculate that the mannose residues on the α1,6-linked mannosyl branch (M4) bind to the enzyme at the same site and in the same manner as mannose-like inhibitor DMNJ. Coordination of the zinc ion with the O2 and O3 hydroxyl oxygens thereby contributes to the enzyme's specificity for mannose. Four acidic amino acid residues, Asp-92, Asp-204, Asp-341 and Asp-472, are candidates for catalytic side chains based on their proximity to the active site (FIG. 10C). Results from a recent study on the mechanism of catalysis in Jack-bean α-mannosidase by Withers and co-workers, using reagents that trap the glycosyl-enzyme intermediate, identified an aspartate residue as the catalytic nucleophile in that enzyme (Howard et al., 1998). Comparison of the highly conserved sequence region surrounding this aspartate in Jack-bean α-mannosidase with the same sequence region in dGMII suggests that aspartate residue 204 in dGMII is the catalytic nucleophile that attacks the glycosidic linkage. For this reaction it is required that Asp-204 is close to the anomeric carbon of the mannose substrate. In the dGMII-DMNJ complex, however, the equivalent anomeric carbon is located 4.6 Å from the nucleophile. Binding of the C2 and C3 substituent hydroxyl oxygens of the flattened five-membered ring in swainsonine causes the inhibitor molecule to tilt, bringing its bridgehead nitrogen N4, in the analogous position to C1 in the substrate, significantly closer to the putative nucleophilic Asp-204 (3.2 Å). This tilted binding mode, stabilized by a hydrogen bond between N4 and Asp-204 and by van der Waals stacking interactions between the 6-membered ring of swainsonine and Phe-206, may resemble the mode of binding of the ring-flattened transition state mannosyl cation. Thus, Phe-206 would stabilize the transition state by compensating for the loss of stacking interactions of the substrate with Trp-95. The highly complementary shape of swainsonine with the active site of dGMII, and its structural analogy with the skewed boat transition state conformation, could therefore explain its 10,000 times higher binding affinity for the enzyme, compared to the substrate-mimic DMNJ (data not shown).

The OD1 oxygen of Asp-204, the putative nucleophile, directly coordinates the zinc ion, implicating a role for the zinc in positioning the nucleophile and in the stabilization of protonation states of the reacting partners. It is tempting to speculate that the change of zinc coordination from T5 to the less favored T6 state (Alberts et al., 1998) on substrate binding may also contribute to the mechanism. From the Tris and DMNJ structures, it is predicted that the coordination would revert to T5 on product release. If so, this transition may energetically facilitate the deglycosylation step. Such evidence of direct zinc involvement in the catalytic mechanism of a glycosyl hydrolase is unprecedented. Arg-288 positions Asp-204 for nucleophilic attack by virtue of hydrogen bond interactions between its NE and NH2 nitrogens and the OD2 oxygen of Asp-204 (FIG. 10C). Based on the expected distance between the two catalytic residues (˜5.5 Å, Davies and Henrissat, 1995) likely candidates for the catalytic base are Asp-341 and Asp-472 (preliminary indications are that the D341N mutant is catalytically inactive, DAK unpublished results). Recent data suggest that other residues, such as tyrosines, possibly play a role in glycosidic bond cleavage (Davies and Henrissat, 1995). Tyrosine residues 269 are 727 are positioned to help stabilize the transition state.

Substrate Binding and Cleavage

The function of GMII is dependent on the presence of β1,2-GlcNAc (G3, FIG. 8A), added to α1,3-linked mannose (M5) by GlcNAc transferase I (see reviews: Kornfeld and Kornfeld, 1985; Moremen et al., 1994). This β1,2-GlcNAc dependence suggests the presence of an additional saccharide-binding site in GMII. Evidence for such a binding site is provided by the observation of an MPD molecule in the structure of dGMII, in the vicinity of the active site cavity. MPD was used as a cryo-protectant during the procedure of flash-freezing of the crystal, prior to data collection (see experimental procedures). The replacement of MPD by the alternative cryo-protectant glycerol resulted in the occupation of this same position by a glycerol molecule. Glycerol has been shown to mimic saccharide binding in structures of glycosyl hydrolases (Schmidt et al., 1998, Vallée et al., 2000).

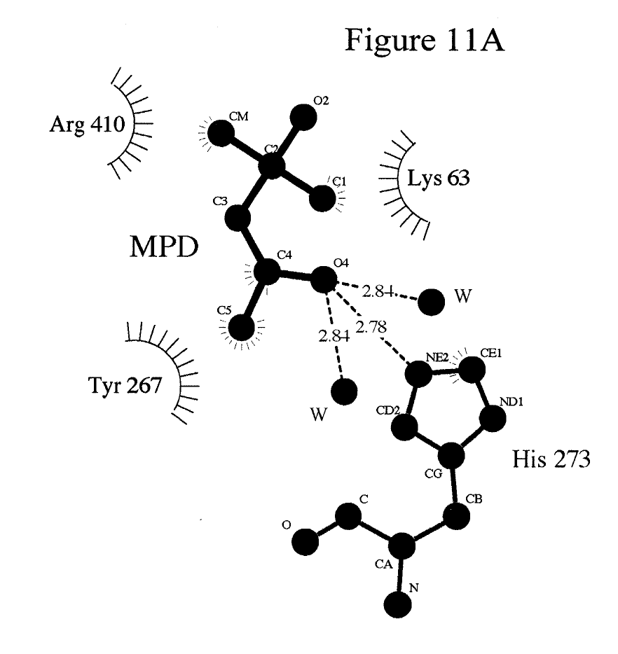

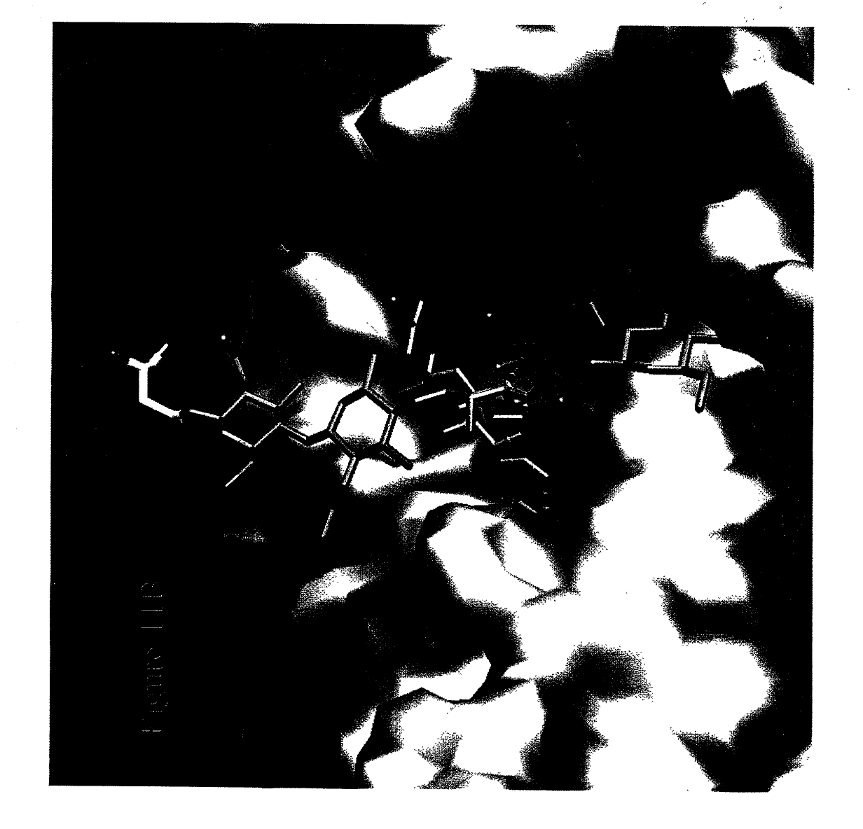

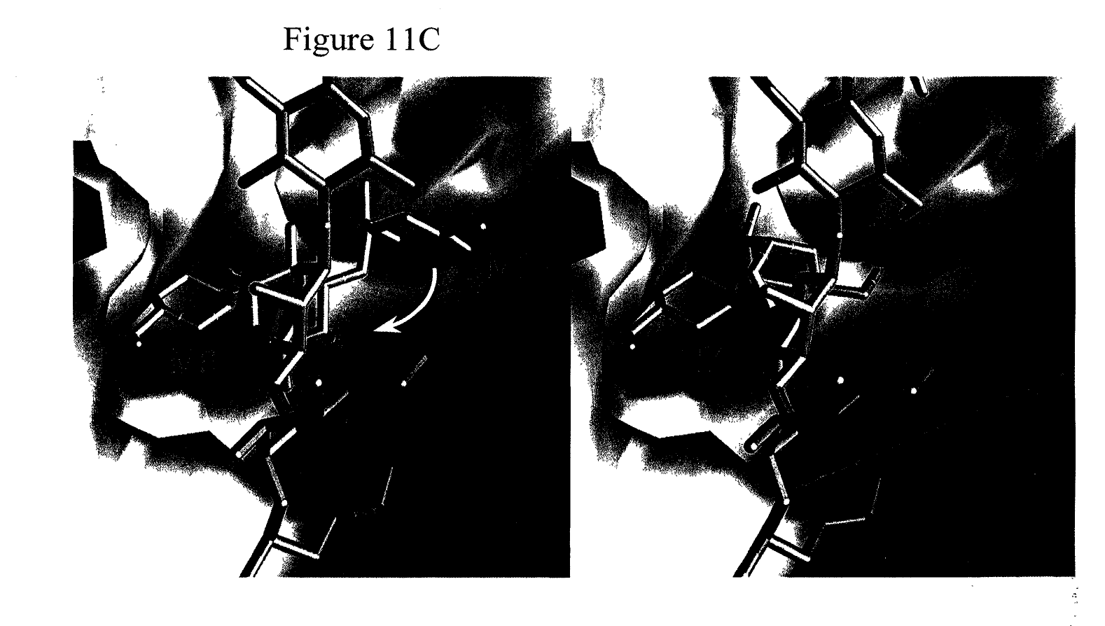

The observation of the binding of MPD and glycerol near dGMII's active site (FIG. 11A) enables a hypothesis regarding the binding and cleavage of α1,6 and α1,3-linked mannoses on the α1,6-linked mannose branch of the GlcNAcMan₅GlcNAc₂ oligosaccharide. In this hypothesis, the MPD binding site is suggested to be the putative site of interaction for β1,2-GlcNAc (G3, FIG. 8A), enabling anchoring of the oligosaccharide substrate in the conserved negatively charged pocket. In FIG. 11B a model is shown of a GlcNAcMan₅GlcNAc₂ structure with the β1,2-GlcNAc residue placed in the MPD binding site and the α1,6-linked M6 mannose docked into the active site, with its hydroxyl oxygens O2 and O3 coordinating the zinc ion. As required, the asparagine linked β1,4-GlcNAc residues G1 and G2 extend away from the surface of the molecule (into the Golgi lumen). Both M4 and the second substrate α1,3-linked M7 mannose are located within the conserved negatively charged pocket pointing away from the active site cavity. In this orientation it can be easily visualized that after cleavage of the α1,6-linked M6 the second, α1,3-linked M7 can be brought into the active site cavity by a ˜180° rotation, through the extended pocket, around the flexible α1,6-linkage of M4 (see FIG. 11C). In addition to the dependence of GMII's action on the presence of the G3 β1,2-GlcNAc, this model provides a mechanism for the cleavage of both mannose residues without major conformational change of the enzyme, and more importantly, without release of the polypeptide-carbohydrate complex, anchored by the stationary GlcNAc, between the two cleavage events. Finally, this model suggests that the α1,6-linked M6 mannose is preferentially cleaved first, enabling the shorter α1,3-linked M7 residue to rotate through the pocket with minimal steric hindrance; according to our model, the proposed ‘swivel’ mechanism would be slightly hampered should the M7 mannose be cleaved first. This is supported by data reported for α-mannosidase II from mung bean seedlings, Xenopus liver, Rat liver Golgi and for enzyme-activity in homogenates of insect cells, showing preferential hydrolytic activity on the M6 mannosyl residue (Kaushal et al., 1990; Altmann and Martz, 1995; Ren et al., 1997).

Conclusions

The structure of the catalytic domain of Golgi α-mannosidase II provides the basis for its zinc ion mediated specificity for mannose, as well as insight into its reaction mechanism. In addition, the result illustrates the structural basis for the mechanism of inhibition by the anti-cancer agent swainsonine, which we propose mimics aspects of the transition state binding. This understanding is critical for the rational design of swainsonine variants and/or novel mechanism-based compounds as specific α-mannosidase II inhibitors, for the treatment of several forms of cancer. A bound MPD molecule identifies a putative GlcNAc binding pocket, located near the active site and enables a hypothesis explaining the enzyme's dependency on the single GlcNAc substitution of the GlcNAcMan₅GlcNAc₂ substrate for binding. Furthermore, it suggests a novel mechanism for successive hydrolysis of the α1,6 and α1,3-linked mannose residues, resulting in the tri-mannose core glycosyl structure. Finally, it opens the door to the design of novel highly specific inhibitors linking together functional sites in the enzyme.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the above specification are herein incorporated by reference.

TABLE 1
Structural coordinates of a Drosophila Golgi α-mannosidase II.

REMARK coordinates from simulated annealing refinement
REMARK refinement resolution: 500.0 − 1.4 A
REMARK starting r = 0.1816 free_r = 0.2003
REMARK final r = 0.1894 free_r = 0.2063
REMARK rmsd bonds = 0.004594 rmsd angles = 1.32379
REMARK wa_initial = 0.264577 wa_dynamics = 0.28954 wa_final = 0.28836
REMARK target = mlf md-method = torsion annealing schedule = slowcool
REMARK starting temperature = 1000 total md steps = 40 * 6
REMARK sg = P2 (1) 2 (1) 2 (1) a = 68.865 b = 109.718 c = 138.599
alpha = 90 beta = 90 gamma = 90
REMARK parameter file 1 CNS_TOPPAR:protein_rep.param
REMARK parameter file 2 CNS_TOPPAR:water_rep.param
REMARK parameter file 3 CNS_TOPPAR:ion.param
REMARK parameter file 4 trs.par
REMARK parameter file 5 mpd.par
REMARK parameter file 6 cis_peptide.param
REMARK parameter file 7 CNS_TOPPAR:carbohydrate.param
REMARK molecular structure file: dgm11cgen.mtf
REMARK input coordinates: dgm11cgen.pdb
REMARK reflection file = ../semetHiR.cv
REMARK ncs = none
REMARK B-correction resolution: 6.0 − 1.4
REMARK initial B-factor correction applied to fobs:
REMARK B11 = 0.609 B22 = −0.765 B33 = 0.155
REMARK B12 = 0.000 B13 = 0.000 B23 = 0.000
REMARK B-factor correction applied to coordinate array B: 0.042
REMARK bulk solvent: density level = 0.35999 e/A\hat{ }3, B-factor = 42.8385 A\hat{ }2
REMARK reflections with |Fobs|/sigma_F < 0.0 rejected
REMARK reflections with |Fobs| > 10000 * rms (Fobs) rejected

| | | |
|---|---|---|
| REMARK theoretical total number of refl. in resol. range: | 206243 | (100.0%) |
| REMARK number of unobserved reflections (no entry or \|F\| = 0): | 59797 | (29.0%) |
| REMARK number of reflections rejected: | 0 | (0.0%) |
| REMARK total number of reflections used: | 146446 | (71.0%) |
| REMARK number of reflections in working set: | 139067 | (67.4%) |
| REMARK number of reflections in test set: | 7379 | (3.6%) |

CRYST1 68.865 109.718 138.599 90.00 90.00 90.00 P 21 21 21
REMARK FILENAME = “dgm11can2_1.pdb”
REMARK DATE: 13-Jul-2000 03:16:24 created by user: jvdelsen
REMARK VERSION: 0.9a

| | | | | | | | | | | | |
|---|---|---|---|---|---|---|---|---|---|---|---|
| ATOM | 1 | C | CYS | A | 31 | 41.938 | 37.136 | −18.751 | 1.00 | 22.64 | A |
| ATOM | 2 | O | CYS | A | 31 | 41.423 | 36.540 | −19.699 | 1.00 | 22.74 | A |
| ATOM | 3 | CB | CYS | A | 31 | 43.833 | 38.473 | −19.585 | 1.00 | 22.94 | A |
| ATOM | 4 | SG | CYS | A | 31 | 45.532 | 39.110 | −19.452 | 1.00 | 23.66 | A |
| ATOM | 5 | N | CYS | A | 31 | 44.185 | 36.072 | −19.063 | 1.00 | 24.03 | A |
| ATOM | 6 | CA | CYS | A | 31 | 43.449 | 37.310 | −18.673 | 1.00 | 23.18 | A |
| ATOM | 7 | N | GLN | A | 32 | 41.229 | 37.666 | −17.760 | 1.00 | 22.29 | A |
| ATOM | 8 | CA | GLN | A | 32 | 39.775 | 37.591 | −17.754 | 1.00 | 22.06 | A |
| ATOM | 9 | CB | GLN | A | 32 | 39.196 | 38.061 | −16.417 | 1.00 | 24.01 | A |
| ATOM | 10 | CG | GLN | A | 32 | 39.409 | 37.126 | −15.248 | 1.00 | 27.12 | A |
| ATOM | 11 | CD | GLN | A | 32 | 38.470 | 37.442 | −14.098 | 1.00 | 28.66 | A |
| ATOM | 12 | OE1 | GLN | A | 32 | 37.252 | 37.307 | −14.224 | 1.00 | 29.78 | A |
| ATOM | 13 | NE2 | GLN | A | 32 | 39.031 | 37.872 | −12.973 | 1.00 | 29.39 | A |
| ATOM | 14 | C | GLN | A | 32 | 39.230 | 38.498 | −18.848 | 1.00 | 20.52 | A |
| ATOM | 15 | O | GLN | A | 32 | 39.817 | 39.534 | −19.162 | 1.00 | 19.74 | A |
| ATOM | 16 | N | ASP | A | 33 | 38.109 | 38.096 | −19.432 | 1.00 | 19.11 | A |
| ATOM | 17 | CA | ASP | A | 33 | 37.460 | 38.885 | −20.470 | 1.00 | 17.47 | A |
| ATOM | 18 | CB | ASP | A | 33 | 36.651 | 37.955 | −21.384 | 1.00 | 18.06 | A |
| ATOM | 19 | CG | ASP | A | 33 | 35.993 | 38.681 | −22.540 | 1.00 | 19.46 | A |
| ATOM | 20 | OD1 | ASP | A | 33 | 35.738 | 38.020 | −23.570 | 1.00 | 20.28 | A |
| ATOM | 21 | OD2 | ASP | A | 33 | 35.714 | 39.893 | −22.421 | 1.00 | 19.63 | A |
| ATOM | 22 | C | ASP | A | 33 | 36.554 | 39.839 | −19.692 | 1.00 | 16.94 | A |
| ATOM | 23 | O | ASP | A | 33 | 35.614 | 39.407 | −19.033 | 1.00 | 18.06 | A |
| ATOM | 24 | N | VAL | A | 34 | 36.854 | 41.132 | −19.748 | 1.00 | 13.82 | A |
| ATOM | 25 | CA | VAL | A | 34 | 36.076 | 42.121 | −19.007 | 1.00 | 12.33 | A |
| ATOM | 26 | CB | VAL | A | 34 | 36.982 | 43.291 | −18.536 | 1.00 | 11.75 | A |
| ATOM | 27 | CG1 | VAL | A | 34 | 38.158 | 42.744 | −17.744 | 1.00 | 12.48 | A |
| ATOM | 28 | CG2 | VAL | A | 34 | 37.485 | 44.093 | −19.737 | 1.00 | 11.56 | A |
| ATOM | 29 | C | VAL | A | 34 | 34.912 | 42.692 | −19.806 | 1.00 | 11.58 | A |
| ATOM | 30 | O | VAL | A | 34 | 34.227 | 43.610 | −19.350 | 1.00 | 11.18 | A |
| ATOM | 31 | N | VAL | A | 35 | 34.668 | 42.129 | −20.986 | 1.00 | 11.40 | A |
| ATOM | 32 | CA | VAL | A | 35 | 33.601 | 42.623 | −21.847 | 1.00 | 11.88 | A |
| ATOM | 33 | CB | VAL | A | 35 | 34.164 | 43.016 | −23.234 | 1.00 | 11.64 | A |
| ATOM | 34 | CG1 | VAL | A | 35 | 33.031 | 43.463 | −24.159 | 1.00 | 11.96 | A |
| ATOM | 35 | CG2 | VAL | A | 35 | 35.199 | 44.113 | −23.082 | 1.00 | 11.54 | A |
| ATOM | 36 | C | VAL | A | 35 | 32.422 | 41.689 | −22.106 | 1.00 | 12.53 | A |
| ATOM | 37 | O | VAL | A | 35 | 31.268 | 42.100 | −22.012 | 1.00 | 12.16 | A |
| ATOM | 38 | N | GLN | A | 36 | 32.719 | 40.434 | −22.422 | 1.00 | 13.91 | A |
| ATOM | 39 | CA | GLN | A | 36 | 31.685 | 39.471 | −22.796 | 1.00 | 15.60 | A |
| ATOM | 40 | CB | GLN | A | 36 | 32.217 | 38.631 | −23.955 | 1.00 | 15.34 | A |
| ATOM | 41 | CG | GLN | A | 36 | 32.986 | 39.457 | −24.972 | 1.00 | 16.14 | A |
| ATOM | 42 | CD | GLN | A | 36 | 33.458 | 38.641 | −26.151 | 1.00 | 16.81 | A |
| ATOM | 43 | OE1 | GLN | A | 36 | 32.699 | 38.388 | −27.084 | 1.00 | 18.19 | A |
| ATOM | 44 | NE2 | GLN | A | 36 | 34.714 | 38.214 | −26.110 | 1.00 | 16.46 | A |
| ATOM | 45 | C | GLN | A | 36 | 31.077 | 38.548 | −21.748 | 1.00 | 17.41 | A |
| ATOM | 46 | O | GLN | A | 36 | 30.128 | 37.825 | −22.048 | 1.00 | 19.27 | A |
| ATOM | 47 | N | ASP | A | 37 | 31.609 | 38.557 | −20.532 | 1.00 | 18.41 | A |
| ATOM | 48 | CA | ASP | A | 37 | 31.080 | 37.700 | −19.475 | 1.00 | 18.97 | A |
| ATOM | 49 | CB | ASP | A | 37 | 32.190 | 36.818 | −18.885 | 1.00 | 21.17 | A |
| ATOM | 50 | CG | ASP | A | 37 | 32.759 | 35.834 | −19.891 | 1.00 | 22.94 | A |
| ATOM | 51 | OD1 | ASP | A | 37 | 31.965 | 35.181 | −20.599 | 1.00 | 24.67 | A |
| ATOM | 52 | OD2 | ASP | A | 37 | 34.000 | 35.707 | −19.963 | 1.00 | 24.47 | A |
| ATOM | 53 | C | ASP | A | 37 | 30.444 | 38.513 | −18.351 | 1.00 | 18.35 | A |
| ATOM | 54 | O | ASP | A | 37 | 31.145 | 39.128 | −17.548 | 1.00 | 20.02 | A |
| ATOM | 55 | N | VAL | A | 38 | 29.117 | 38.511 | −18.294 | 1.00 | 17.12 | A |
| ATOM | 56 | CA | VAL | A | 38 | 28.404 | 39.242 | −17.250 | 1.00 | 16.29 | A |
| ATOM | 57 | CB | VAL | A | 38 | 26.902 | 39.362 | −17.579 | 1.00 | 16.90 | A |
| ATOM | 58 | CG1 | VAL | A | 38 | 26.180 | 40.114 | −16.468 | 1.00 | 16.50 | A |
| ATOM | 59 | CG2 | VAL | A | 38 | 26.721 | 40.074 | −18.915 | 1.00 | 16.46 | A |
| ATOM | 60 | C | VAL | A | 38 | 28.559 | 38.512 | −15.917 | 1.00 | 15.90 | A |
| ATOM | 61 | O | VAL | A | 38 | 28.108 | 37.377 | −15.763 | 1.00 | 15.86 | A |
| ATOM | 62 | N | PRO | A | 39 | 29.204 | 39.157 | −14.933 | 1.00 | 15.02 | A |
| ATOM | 63 | CD | PRO | A | 39 | 29.892 | 40.459 | −14.998 | 1.00 | 14.29 | A |
| ATOM | 64 | CA | PRO | A | 39 | 29.399 | 38.531 | −13.623 | 1.00 | 14.60 | A |
| ATOM | 65 | CB | PRO | A | 39 | 30.154 | 39.603 | −12.836 | 1.00 | 14.74 | A |
| ATOM | 66 | CG | PRO | A | 39 | 30.927 | 40.321 | −13.901 | 1.00 | 13.83 | A |
| ATOM | 67 | C | PRO | A | 39 | 28.089 | 38.150 | −12.949 | 1.00 | 14.68 | A |
| ATOM | 68 | O | PRO | A | 39 | 27.092 | 38.866 | −13.045 | 1.00 | 14.60 | A |
| ATOM | 69 | N | ASN | A | 40 | 28.095 | 37.009 | −12.270 | 1.00 | 15.48 | A |
| ATOM | 70 | CA | ASN | A | 40 | 26.913 | 36.557 | −11.557 | 1.00 | 16.28 | A |
| ATOM | 71 | CB | ASN | A | 40 | 26.727 | 35.048 | −11.725 | 1.00 | 18.96 | A |
| ATOM | 72 | CG | ASN | A | 40 | 25.614 | 34.501 | −10.855 | 1.00 | 21.26 | A |
| ATOM | 73 | OD1 | ASN | A | 40 | 24.500 | 35.026 | −10.847 | 1.00 | 24.33 | A |
| ATOM | 74 | ND2 | ASN | A | 40 | 25.909 | 33.437 | −10.119 | 1.00 | 23.94 | A |
| ATOM | 75 | C | ASN | A | 40 | 27.113 | 36.899 | −10.089 | 1.00 | 15.67 | A |
| ATOM | 76 | O | ASN | A | 40 | 27.902 | 36.259 | −9.396 | 1.00 | 15.97 | A |
| ATOM | 77 | N | VAL | A | 41 | 26.408 | 37.922 | −9.623 | 1.00 | 14.19 | A |
| ATOM | 78 | CA | VAL | A | 41 | 26.522 | 38.347 | −8.232 | 1.00 | 12.77 | A |
| ATOM | 79 | CB | VAL | A | 41 | 27.144 | 39.764 | −8.126 | 1.00 | 11.76 | A |
| ATOM | 80 | CG1 | VAL | A | 41 | 28.584 | 39.738 | −8.606 | 1.00 | 12.95 | A |
| ATOM | 81 | CG2 | VAL | A | 41 | 26.328 | 40.755 | −8.946 | 1.00 | 12.22 | A |
| ATOM | 82 | C | VAL | A | 41 | 25.162 | 38.353 | −7.547 | 1.00 | 12.71 | A |
| ATOM | 83 | O | VAL | A | 41 | 24.125 | 38.479 | −8.198 | 1.00 | 13.39 | A |
| ATOM | 84 | N | ASP | A | 42 | 25.171 | 38.217 | −6.226 | 1.00 | 11.92 | A |
| ATOM | 85 | CA | ASP | A | 42 | 23.934 | 38.211 | −5.461 | 1.00 | 12.46 | A |
| ATOM | 86 | CB | ASP | A | 42 | 24.210 | 37.791 | −4.017 | 1.00 | 13.55 | A |
| ATOM | 87 | CG | ASP | A | 42 | 24.708 | 36.366 | −3.913 | 1.00 | 14.63 | A |
| ATOM | 88 | OD1 | ASP | A | 42 | 24.009 | 35.457 | −4.416 | 1.00 | 16.11 | A |
| ATOM | 89 | OD2 | ASP | A | 42 | 25.788 | 36.144 | −3.327 | 1.00 | 14.87 | A |
| ATOM | 90 | C | ASP | A | 42 | 23.261 | 39.576 | −5.480 | 1.00 | 11.96 | A |
| ATOM | 91 | O | ASP | A | 42 | 22.036 | 39.675 | −5.540 | 1.00 | 12.50 | A |
| ATOM | 92 | N | VAL | A | 43 | 24.066 | 40.632 | −5.419 | 1.00 | 10.49 | A |
| ATOM | 93 | CA | VAL | A | 43 | 23.537 | 41.987 | −5.434 | 1.00 | 10.78 | A |
| ATOM | 94 | CB | VAL | A | 43 | 23.693 | 42.686 | −4.056 | 1.00 | 10.05 | A |
| ATOM | 95 | CG1 | VAL | A | 43 | 23.072 | 44.078 | −4.105 | 1.00 | 10.79 | A |
| ATOM | 96 | CG2 | VAL | A | 43 | 23.042 | 41.848 | −2.955 | 1.00 | 10.64 | A |
| ATOM | 97 | C | VAL | A | 43 | 24.291 | 42.806 | −6.471 | 1.00 | 9.86 | A |
| ATOM | 98 | O | VAL | A | 43 | 25.515 | 42.922 | −6.414 | 1.00 | 10.17 | A |
| ATOM | 99 | N | GLN | A | 44 | 23.559 | 43.336 | −7.443 | 1.00 | 9.59 | A |
| ATOM | 100 | CA | GLN | A | 44 | 24.158 | 44.174 | −8.471 | 1.00 | 9.74 | A |
| ATOM | 101 | CB | GLN | A | 44 | 23.920 | 43.576 | −9.860 | 1.00 | 9.50 | A |
| ATOM | 102 | CG | GLN | A | 44 | 24.977 | 43.983 | −10.869 | 1.00 | 9.04 | A |
| ATOM | 103 | CD | GLN | A | 44 | 25.078 | 45.482 | −11.000 | 1.00 | 10.11 | A |
| ATOM | 104 | OE1 | GLN | A | 44 | 24.107 | 46.144 | −11.348 | 1.00 | 10.00 | A |
| ATOM | 105 | NE2 | GLN | A | 44 | 26.255 | 46.028 | −10.708 | 1.00 | 9.62 | A |
| ATOM | 106 | C | GLN | A | 44 | 23.409 | 45.489 | −8.282 | 1.00 | 9.74 | A |
| ATOM | 107 | O | GLN | A | 44 | 22.203 | 45.562 | −8.488 | 1.00 | 9.41 | A |
| ATOM | 108 | N | MSE | A | 45 | 24.125 | 46.526 | −7.866 | 1.00 | 9.19 | A |
| ATOM | 109 | CA | MSE | A | 45 | 23.488 | 47.795 | −7.559 | 1.00 | 9.37 | A |
| ATOM | 110 | CB | MSE | A | 45 | 24.531 | 48.784 | −7.035 | 1.00 | 10.21 | A |
| ATOM | 111 | CG | MSE | A | 45 | 25.149 | 48.353 | −5.699 | 1.00 | 11.50 | A |
| ATOM | 112 | SE | MSE | A | 45 | 23.842 | 47.932 | −4.319 | 1.00 | 18.05 | A |
| ATOM | 113 | CE | MSE | A | 45 | 23.146 | 49.711 | −4.052 | 1.00 | 15.91 | A |
| ATOM | 114 | C | MSE | A | 45 | 22.606 | 48.459 | −8.606 | 1.00 | 8.92 | A |
| ATOM | 115 | O | MSE | A | 45 | 21.614 | 49.094 | −8.245 | 1.00 | 9.44 | A |
| ATOM | 116 | N | LEU | A | 46 | 22.942 | 48.327 | −9.886 | 1.00 | 9.08 | A |
| ATOM | 117 | CA | LEU | A | 46 | 22.108 | 48.933 | −10.923 | 1.00 | 9.97 | A |
| ATOM | 118 | CB | LEU | A | 46 | 22.793 | 48.872 | −12.294 | 1.00 | 10.46 | A |
| ATOM | 119 | CG | LEU | A | 46 | 22.050 | 49.591 | −13.430 | 1.00 | 10.75 | A |
| ATOM | 120 | CD1 | LEU | A | 46 | 22.054 | 51.101 | −13.186 | 1.00 | 11.00 | A |
| ATOM | 121 | CD2 | LEU | A | 46 | 22.706 | 49.268 | −14.760 | 1.00 | 10.73 | A |
| ATOM | 122 | C | LEU | A | 46 | 20.783 | 48.174 | −10.974 | 1.00 | 10.31 | A |
| ATOM | 123 | O | LEU | A | 46 | 19.716 | 48.773 | −11.109 | 1.00 | 9.89 | A |
| ATOM | 124 | N | GLU | A | 47 | 20.860 | 46.853 | −10.848 | 1.00 | 11.31 | A |
| ATOM | 125 | CA | GLU | A | 47 | 19.662 | 46.020 | −10.880 | 1.00 | 12.80 | A |
| ATOM | 126 | CB | GLU | A | 47 | 20.050 | 44.538 | −10.943 | 1.00 | 13.61 | A |
| ATOM | 127 | CG | GLU | A | 47 | 18.875 | 43.590 | −11.181 | 1.00 | 16.25 | A |
| ATOM | 128 | CD | GLU | A | 47 | 18.100 | 43.257 | −9.920 | 1.00 | 18.05 | A |
| ATOM | 129 | OE1 | GLU | A | 47 | 16.963 | 42.754 | −10.042 | 1.00 | 20.21 | A |
| ATOM | 130 | OE2 | GLU | A | 47 | 18.622 | 43.479 | −8.809 | 1.00 | 18.69 | A |
| ATOM | 131 | C | GLU | A | 47 | 18.810 | 46.295 | −9.648 | 1.00 | 12.80 | A |
| ATOM | 132 | O | GLU | A | 47 | 17.586 | 46.385 | −9.736 | 1.00 | 12.50 | A |
| ATOM | 133 | N | LEU | A | 48 | 19.460 | 46.444 | −8.499 | 1.00 | 12.14 | A |
| ATOM | 134 | CA | LEU | A | 48 | 18.740 | 46.716 | −7.263 | 1.00 | 12.63 | A |
| ATOM | 135 | CB | LEU | A | 48 | 19.705 | 46.729 | −6.076 | 1.00 | 14.14 | A |
| ATOM | 136 | CG | LEU | A | 48 | 19.055 | 46.870 | −4.697 | 1.00 | 15.25 | A |
| ATOM | 137 | CD1 | LEU | A | 48 | 18.053 | 45.745 | −4.474 | 1.00 | 16.69 | A |
| ATOM | 138 | CD2 | LEU | A | 48 | 20.130 | 46.847 | −3.620 | 1.00 | 16.69 | A |
| ATOM | 139 | C | LEU | A | 48 | 18.019 | 48.057 | −7.367 | 1.00 | 12.34 | A |
| ATOM | 140 | O | LEU | A | 48 | 16.863 | 48.187 | −6.966 | 1.00 | 12.49 | A |
| ATOM | 141 | N | TYR | A | 49 | 18.704 | 49.054 | −7.918 | 1.00 | 11.81 | A |
| ATOM | 142 | CA | TYR | A | 49 | 18.113 | 50.376 | −8.081 | 1.00 | 12.23 | A |
| ATOM | 143 | CB | TYR | A | 49 | 19.149 | 51.349 | −8.666 | 1.00 | 11.90 | A |
| ATOM | 144 | CG | TYR | A | 49 | 19.709 | 52.296 | −7.630 | 1.00 | 10.96 | A |
| ATOM | 145 | CD1 | TYR | A | 49 | 20.175 | 51.815 | −6.403 | 1.00 | 10.54 | A |
| ATOM | 146 | CE1 | TYR | A | 49 | 20.626 | 52.679 | −5.416 | 1.00 | 10.82 | A |
| ATOM | 147 | CD2 | TYR | A | 49 | 19.719 | 53.674 | −7.847 | 1.00 | 10.08 | A |
| ATOM | 148 | CE2 | TYR | A | 49 | 20.170 | 54.552 | −6.863 | 1.00 | 10.64 | A |
| ATOM | 149 | CZ | TYR | A | 49 | 20.617 | 54.046 | −5.649 | 1.00 | 9.91 | A |
| ATOM | 150 | OH | TYR | A | 49 | 21.025 | 54.902 | −4.653 | 1.00 | 10.90 | A |
| ATOM | 151 | C | TYR | A | 49 | 16.874 | 50.330 | −8.972 | 1.00 | 12.98 | A |
| ATOM | 152 | O | TYR | A | 49 | 15.900 | 51.047 | −8.740 | 1.00 | 13.38 | A |
| ATOM | 153 | N | ASP | A | 50 | 16.910 | 49.470 | −9.983 | 1.00 | 13.83 | A |
| ATOM | 154 | CA | ASP | A | 50 | 15.793 | 49.339 | −10.910 | 1.00 | 15.90 | A |
| ATOM | 155 | CB | ASP | A | 50 | 16.187 | 48.407 | −12.060 | 1.00 | 16.89 | A |
| ATOM | 156 | CG | ASP | A | 50 | 15.410 | 48.680 | −13.334 | 1.00 | 18.91 | A |
| ATOM | 157 | OD1 | ASP | A | 50 | 15.524 | 47.869 | −14.279 | 1.00 | 21.08 | A |
| ATOM | 158 | OD2 | ASP | A | 50 | 14.698 | 49.705 | −13.401 | 1.00 | 19.63 | A |
| ATOM | 159 | C | ASP | A | 50 | 14.555 | 48.792 | −10.194 | 1.00 | 16.66 | A |
| ATOM | 160 | O | ASP | A | 50 | 13.430 | 49.204 | −10.484 | 1.00 | 16.29 | A |
| ATOM | 161 | N | ARG | A | 51 | 14.772 | 47.882 | −9.248 | 1.00 | 17.87 | A |
| ATOM | 162 | CA | ARG | A | 51 | 13.678 | 47.256 | −8.501 | 1.00 | 19.68 | A |
| ATOM | 163 | CB | ARG | A | 51 | 14.072 | 45.836 | −8.075 | 1.00 | 22.37 | A |
| ATOM | 164 | CG | ARG | A | 51 | 14.210 | 44.853 | −9.219 | 1.00 | 26.10 | A |
| ATOM | 165 | CD | ARG | A | 51 | 14.327 | 43.409 | −8.730 | 1.00 | 29.22 | A |
| ATOM | 166 | NE | ARG | A | 51 | 15.547 | 43.159 | −7.964 | 1.00 | 31.13 | A |
| ATOM | 167 | CZ | ARG | A | 51 | 15.681 | 43.377 | −6.659 | 1.00 | 32.25 | A |
| ATOM | 168 | NH1 | ARG | A | 51 | 14.665 | 43.851 | −5.950 | 1.00 | 33.45 | A |
| ATOM | 169 | NH2 | ARG | A | 51 | 16.838 | 43.121 | −6.063 | 1.00 | 32.60 | A |
| ATOM | 170 | C | ARG | A | 51 | 13.182 | 48.008 | −7.267 | 1.00 | 19.33 | A |
| ATOM | 171 | O | ARG | A | 51 | 11.999 | 47.938 | −6.935 | 1.00 | 20.01 | A |
| ATOM | 172 | N | MSE | A | 52 | 14.080 | 48.708 | −6.582 | 1.00 | 19.03 | A |
| ATOM | 173 | CA | MSE | A | 52 | 13.722 | 49.450 | −5.374 | 1.00 | 19.27 | A |
| ATOM | 174 | CB | MSE | A | 52 | 14.976 | 50.053 | −4.742 | 1.00 | 20.66 | A |
| ATOM | 175 | CG | MSE | A | 52 | 15.912 | 49.042 | −4.122 | 1.00 | 22.65 | A |
| ATOM | 176 | SE | MSE | A | 52 | 17.569 | 49.884 | −3.600 | 1.00 | 27.63 | A |
| ATOM | 177 | CE | MSE | A | 52 | 16.866 | 51.229 | −2.405 | 1.00 | 24.75 | A |
| ATOM | 178 | C | MSE | A | 52 | 12.698 | 50.557 | −5.597 | 1.00 | 18.62 | A |
| ATOM | 179 | O | MSE | A | 52 | 12.690 | 51.209 | −6.640 | 1.00 | 18.85 | A |
| ATOM | 180 | N | SER | A | 53 | 11.850 | 50.777 | −4.595 | 1.00 | 18.04 | A |
| ATOM | 181 | CA | SER | A | 53 | 10.814 | 51.805 | −4.673 | 1.00 | 18.07 | A |
| ATOM | 182 | CB | SER | A | 53 | 9.525 | 51.291 | −4.028 | 1.00 | 19.37 | A |
| ATOM | 183 | OG | SER | A | 53 | 9.062 | 50.124 | −4.689 | 1.00 | 21.66 | A |
| ATOM | 184 | C | SER | A | 53 | 11.233 | 53.118 | −4.010 | 1.00 | 17.33 | A |
| ATOM | 185 | O | SER | A | 53 | 10.615 | 54.158 | −4.235 | 1.00 | 17.18 | A |
| ATOM | 186 | N | PHE | A | 54 | 12.276 | 53.058 | −3.188 | 1.00 | 16.61 | A |
| ATOM | 187 | CA | PHE | A | 54 | 12.800 | 54.229 | −2.488 | 1.00 | 16.04 | A |
| ATOM | 188 | CB | PHE | A | 54 | 13.474 | 55.196 | −3.474 | 1.00 | 15.36 | A |
| ATOM | 189 | CG | PHE | A | 54 | 14.708 | 54.642 | −4.140 | 1.00 | 13.72 | A |
| ATOM | 190 | CD1 | PHE | A | 54 | 14.604 | 53.800 | −5.242 | 1.00 | 13.06 | A |
| ATOM | 191 | CD2 | PHE | A | 54 | 15.973 | 54.979 | −3.672 | 1.00 | 13.80 | A |
| ATOM | 192 | CE1 | PHE | A | 54 | 15.745 | 53.301 | −5.874 | 1.00 | 13.32 | A |
| ATOM | 193 | CE2 | PHE | A | 54 | 17.121 | 54.488 | −4.294 | 1.00 | 13.91 | A |
| ATOM | 194 | CZ | PHE | A | 54 | 17.006 | 53.646 | −5.400 | 1.00 | 13.56 | A |
| ATOM | 195 | C | PHE | A | 54 | 11.760 | 55.008 | −1.680 | 1.00 | 16.83 | A |
| ATOM | 196 | O | PHE | A | 54 | 11.858 | 56.228 | −1.555 | 1.00 | 16.55 | A |
| ATOM | 197 | N | LYS | A | 55 | 10.768 | 54.319 | −1.126 | 1.00 | 17.41 | A |
| ATOM | 198 | CA | LYS | A | 55 | 9.757 | 55.016 | −0.337 | 1.00 | 19.14 | A |
| ATOM | 199 | CB | LYS | A | 55 | 8.554 | 54.107 | −0.078 | 1.00 | 19.64 | A |
| ATOM | 200 | CG | LYS | A | 55 | 7.836 | 53.664 | −1.343 | 1.00 | 20.85 | A |
| ATOM | 201 | CD | LYS | A | 55 | 7.418 | 54.851 | −2.206 | 1.00 | 22.09 | A |
| ATOM | 202 | CE | LYS | A | 55 | 6.676 | 54.383 | −3.454 | 1.00 | 23.32 | A |
| ATOM | 203 | NZ | LYS | A | 55 | 6.271 | 55.511 | −4.340 | 1.00 | 24.17 | A |
| ATOM | 204 | C | LYS | A | 55 | 10.354 | 55.486 | 0.985 | 1.00 | 19.50 | A |
| ATOM | 205 | O | LYS | A | 55 | 11.028 | 54.726 | 1.679 | 1.00 | 20.05 | A |
| ATOM | 206 | N | ASP | A | 56 | 10.102 | 56.745 | 1.324 | 1.00 | 19.60 | A |
| ATOM | 207 | CA | ASP | A | 56 | 10.622 | 57.341 | 2.549 | 1.00 | 20.15 | A |
| ATOM | 208 | CB | ASP | A | 56 | 11.139 | 58.753 | 2.240 | 1.00 | 19.39 | A |
| ATOM | 209 | CG | ASP | A | 56 | 11.741 | 59.441 | 3.450 | 1.00 | 19.05 | A |
| ATOM | 210 | OD1 | ASP | A | 56 | 12.273 | 58.744 | 4.338 | 1.00 | 19.04 | A |
| ATOM | 211 | OD2 | ASP | A | 56 | 11.697 | 60.689 | 3.503 | 1.00 | 19.38 | A |
| ATOM | 212 | C | ASP | A | 56 | 9.551 | 57.383 | 3.635 | 1.00 | 20.95 | A |
| ATOM | 213 | O | ASP | A | 56 | 8.972 | 58.432 | 3.912 | 1.00 | 21.55 | A |
| ATOM | 214 | N | ILE | A | 57 | 9.295 | 56.236 | 4.254 | 1.00 | 21.88 | A |
| ATOM | 215 | CA | ILE | A | 57 | 8.283 | 56.158 | 5.301 | 1.00 | 22.85 | A |
| ATOM | 216 | CB | ILE | A | 57 | 7.404 | 54.895 | 5.141 | 1.00 | 24.33 | A |
| ATOM | 217 | CG2 | ILE | A | 57 | 6.848 | 54.823 | 3.723 | 1.00 | 24.60 | A |
| ATOM | 218 | CG1 | ILE | A | 57 | 8.224 | 53.639 | 5.437 | 1.00 | 25.50 | A |
| ATOM | 219 | CD1 | ILE | A | 57 | 7.404 | 52.363 | 5.446 | 1.00 | 26.92 | A |
| ATOM | 220 | C | ILE | A | 57 | 8.897 | 56.146 | 6.695 | 1.00 | 22.59 | A |
| ATOM | 221 | O | ILE | A | 57 | 10.038 | 55.722 | 6.881 | 1.00 | 22.42 | A |
| ATOM | 222 | N | ASP | A | 58 | 8.128 | 56.623 | 7.669 | 1.00 | 22.17 | A |
| ATOM | 223 | CA | ASP | A | 58 | 8.566 | 56.667 | 9.059 | 1.00 | 21.75 | A |
| ATOM | 224 | CB | ASP | A | 58 | 7.605 | 57.538 | 9.873 | 1.00 | 22.45 | A |
| ATOM | 225 | CG | ASP | A | 58 | 8.017 | 57.674 | 11.327 | 1.00 | 23.28 | A |
| ATOM | 226 | OD1 | ASP | A | 58 | 7.417 | 58.514 | 12.033 | 1.00 | 24.70 | A |
| ATOM | 227 | OD2 | ASP | A | 58 | 8.929 | 56.948 | 11.771 | 1.00 | 22.76 | A |
| ATOM | 228 | C | ASP | A | 58 | 8.580 | 55.243 | 9.604 | 1.00 | 21.36 | A |
| ATOM | 229 | O | ASP | A | 58 | 7.528 | 54.637 | 9.801 | 1.00 | 20.84 | A |
| ATOM | 230 | N | GLY | A | 59 | 9.775 | 54.712 | 9.842 | 1.00 | 19.94 | A |
| ATOM | 231 | CA | GLY | A | 59 | 9.887 | 53.355 | 10.346 | 1.00 | 19.09 | A |
| ATOM | 232 | C | GLY | A | 59 | 9.859 | 53.221 | 11.858 | 1.00 | 18.34 | A |
| ATOM | 233 | O | GLY | A | 59 | 10.062 | 52.128 | 12.383 | 1.00 | 18.39 | A |
| ATOM | 234 | N | GLY | A | 60 | 9.605 | 54.321 | 12.559 | 1.00 | 17.94 | A |
| ATOM | 235 | CA | GLY | A | 60 | 9.567 | 54.280 | 14.012 | 1.00 | 17.49 | A |
| ATOM | 236 | C | GLY | A | 60 | 10.801 | 54.927 | 14.615 | 1.00 | 16.45 | A |
| ATOM | 237 | O | GLY | A | 60 | 11.318 | 55.898 | 14.062 | 1.00 | 17.23 | A |
| ATOM | 238 | N | VAL | A | 61 | 11.273 | 54.405 | 15.747 | 1.00 | 16.17 | A |
| ATOM | 239 | CA | VAL | A | 61 | 12.464 | 54.962 | 16.383 | 1.00 | 15.12 | A |
| ATOM | 240 | CB | VAL | A | 61 | 12.836 | 54.209 | 17.683 | 1.00 | 15.83 | A |
| ATOM | 241 | CG1 | VAL | A | 61 | 11.775 | 54.473 | 18.746 | 1.00 | 15.81 | A |
| ATOM | 242 | CG2 | VAL | A | 61 | 12.964 | 52.721 | 17.418 | 1.00 | 16.10 | A |
| ATOM | 243 | C | VAL | A | 61 | 13.618 | 54.907 | 15.385 | 1.00 | 14.48 | A |
| ATOM | 244 | O | VAL | A | 61 | 14.481 | 55.784 | 15.373 | 1.00 | 13.96 | A |
| ATOM | 245 | N | TRP | A | 62 | 13.641 | 53.863 | 14.561 | 1.00 | 14.06 | A |
| ATOM | 246 | CA | TRP | A | 62 | 14.645 | 53.770 | 13.507 | 1.00 | 13.87 | A |
| ATOM | 247 | CB | TRP | A | 62 | 15.020 | 52.316 | 13.194 | 1.00 | 13.36 | A |
| ATOM | 248 | CG | TRP | A | 62 | 15.986 | 52.190 | 12.037 | 1.00 | 12.76 | A |
| ATOM | 249 | CD2 | TRP | A | 62 | 16.349 | 50.990 | 11.343 | 1.00 | 12.39 | A |
| ATOM | 250 | CE2 | TRP | A | 62 | 17.259 | 51.355 | 10.322 | 1.00 | 12.49 | A |
| ATOM | 251 | CE3 | TRP | A | 62 | 15.994 | 49.640 | 11.483 | 1.00 | 11.15 | A |
| ATOM | 252 | CD1 | TRP | A | 62 | 16.676 | 53.205 | 11.427 | 1.00 | 12.72 | A |
| ATOM | 253 | NE1 | TRP | A | 62 | 17.438 | 52.711 | 10.396 | 1.00 | 12.59 | A |
| ATOM | 254 | CZ2 | TRP | A | 62 | 17.817 | 50.420 | 9.444 | 1.00 | 12.40 | A |
| ATOM | 255 | CZ3 | TRP | A | 62 | 16.550 | 48.708 | 10.610 | 1.00 | 12.01 | A |
| ATOM | 256 | CH2 | TRP | A | 62 | 17.452 | 49.105 | 9.602 | 1.00 | 11.99 | A |
| ATOM | 257 | C | TRP | A | 62 | 13.846 | 54.378 | 12.361 | 1.00 | 14.13 | A |
| ATOM | 258 | O | TRP | A | 62 | 13.164 | 53.677 | 11.615 | 1.00 | 14.64 | A |
| ATOM | 259 | N | LYS | A | 63 | 13.923 | 55.700 | 12.255 | 1.00 | 14.09 | A |
| ATOM | 260 | CA | LYS | A | 63 | 13.170 | 56.456 | 11.262 | 1.00 | 14.88 | A |
| ATOM | 261 | CB | LYS | A | 63 | 13.613 | 57.922 | 11.290 | 1.00 | 15.30 | A |
| ATOM | 262 | CG | LYS | A | 63 | 13.218 | 58.676 | 12.559 | 1.00 | 18.82 | A |
| ATOM | 263 | CD | LYS | A | 63 | 11.705 | 58.848 | 12.658 | 1.00 | 20.72 | A |
| ATOM | 264 | CE | LYS | A | 63 | 11.305 | 59.678 | 13.873 | 1.00 | 23.06 | A |
| ATOM | 265 | NZ | LYS | A | 63 | 11.653 | 59.027 | 15.170 | 1.00 | 24.73 | A |
| ATOM | 266 | C | LYS | A | 63 | 13.187 | 55.954 | 9.826 | 1.00 | 14.71 | A |
| ATOM | 267 | O | LYS | A | 63 | 12.175 | 56.038 | 9.129 | 1.00 | 15.50 | A |
| ATOM | 268 | N | GLN | A | 64 | 14.320 | 55.430 | 9.378 | 1.00 | 13.97 | A |
| ATOM | 269 | CA | GLN | A | 64 | 14.419 | 54.964 | 8.001 | 1.00 | 13.67 | A |
| ATOM | 270 | CB | GLN | A | 64 | 15.635 | 55.615 | 7.344 | 1.00 | 13.23 | A |
| ATOM | 271 | CG | GLN | A | 64 | 15.555 | 57.133 | 7.373 | 1.00 | 12.86 | A |
| ATOM | 272 | CD | GLN | A | 64 | 16.908 | 57.787 | 7.211 | 1.00 | 11.84 | A |
| ATOM | 273 | OE1 | GLN | A | 64 | 17.851 | 57.465 | 7.933 | 1.00 | 13.01 | A |
| ATOM | 274 | NE2 | GLN | A | 64 | 17.011 | 58.718 | 6.265 | 1.00 | 12.04 | A |
| ATOM | 275 | C | GLN | A | 64 | 14.472 | 53.449 | 7.856 | 1.00 | 13.24 | A |
| ATOM | 276 | O | GLN | A | 64 | 14.847 | 52.929 | 6.805 | 1.00 | 13.10 | A |
| ATOM | 277 | N | GLY | A | 65 | 14.076 | 52.749 | 8.915 | 1.00 | 13.24 | A |
| ATOM | 278 | CA | GLY | A | 65 | 14.064 | 51.297 | 8.887 | 1.00 | 13.74 | A |
| ATOM | 279 | C | GLY | A | 65 | 12.710 | 50.744 | 9.298 | 1.00 | 14.46 | A |
| ATOM | 280 | O | GLY | A | 65 | 11.687 | 51.085 | 8.703 | 1.00 | 14.40 | A |
| ATOM | 281 | N | TRP | A | 66 | 12.709 | 49.889 | 10.316 | 1.00 | 14.87 | A |
| ATOM | 282 | CA | TRP | A | 66 | 11.482 | 49.283 | 10.830 | 1.00 | 15.41 | A |
| ATOM | 283 | CB | TRP | A | 66 | 11.106 | 48.058 | 9.987 | 1.00 | 15.37 | A |
| ATOM | 284 | CG | TRP | A | 66 | 12.040 | 46.889 | 10.153 | 1.00 | 15.33 | A |
| ATOM | 285 | CD2 | TRP | A | 66 | 13.248 | 46.644 | 9.422 | 1.00 | 15.00 | A |
| ATOM | 286 | CE2 | TRP | A | 66 | 13.804 | 45.445 | 9.923 | 1.00 | 14.98 | A |
| ATOM | 287 | CE3 | TRP | A | 66 | 13.915 | 47.321 | 8.391 | 1.00 | 14.47 | A |
| ATOM | 288 | CD1 | TRP | A | 66 | 11.918 | 45.861 | 11.043 | 1.00 | 15.31 | A |
| ATOM | 289 | NE1 | TRP | A | 66 | 12.972 | 44.988 | 10.911 | 1.00 | 16.13 | A |
| ATOM | 290 | CZ2 | TRP | A | 66 | 14.998 | 44.907 | 9.429 | 1.00 | 14.84 | A |
| ATOM | 291 | CZ3 | TRP | A | 66 | 15.105 | 46.785 | 7.900 | 1.00 | 14.49 | A |
| ATOM | 292 | CH2 | TRP | A | 66 | 15.633 | 45.589 | 8.421 | 1.00 | 14.85 | A |
| ATOM | 293 | C | TRP | A | 66 | 11.751 | 48.864 | 12.271 | 1.00 | 15.70 | A |
| ATOM | 294 | O | TRP | A | 66 | 12.888 | 48.946 | 12.734 | 1.00 | 15.45 | A |
| ATOM | 295 | N | ASN | A | 67 | 10.717 | 48.428 | 12.985 | 1.00 | 16.28 | A |
| ATOM | 296 | CA | ASN | A | 67 | 10.899 | 47.991 | 14.368 | 1.00 | 16.95 | A |
| ATOM | 297 | CB | ASN | A | 67 | 9.564 | 47.958 | 15.119 | 1.00 | 19.00 | A |
| ATOM | 298 | CG | ASN | A | 67 | 8.948 | 49.331 | 15.270 | 1.00 | 20.01 | A |
| ATOM | 299 | OD1 | ASN | A | 67 | 9.638 | 50.304 | 15.574 | 1.00 | 22.02 | A |
| ATOM | 300 | ND2 | ASN | A | 67 | 7.637 | 49.417 | 15.071 | 1.00 | 22.35 | A |
| ATOM | 301 | C | ASN | A | 67 | 11.517 | 46.599 | 14.371 | 1.00 | 16.92 | A |
| ATOM | 302 | O | ASN | A | 67 | 10.837 | 45.604 | 14.111 | 1.00 | 16.93 | A |
| ATOM | 303 | N | ILE | A | 68 | 12.809 | 46.533 | 14.669 | 1.00 | 16.90 | A |
| ATOM | 304 | CA | ILE | A | 68 | 13.518 | 45.262 | 14.681 | 1.00 | 17.11 | A |
| ATOM | 305 | CB | ILE | A | 68 | 15.043 | 45.472 | 14.801 | 1.00 | 16.47 | A |
| ATOM | 306 | CG2 | ILE | A | 68 | 15.759 | 44.130 | 14.753 | 1.00 | 17.44 | A |
| ATOM | 307 | CG1 | ILE | A | 68 | 15.538 | 46.370 | 13.664 | 1.00 | 16.41 | A |
| ATOM | 308 | CD1 | ILE | A | 68 | 17.000 | 46.764 | 13.794 | 1.00 | 15.35 | A |
| ATOM | 309 | C | ILE | A | 68 | 13.066 | 44.365 | 15.824 | 1.00 | 18.00 | A |
| ATOM | 310 | O | ILE | A | 68 | 12.954 | 44.804 | 16.968 | 1.00 | 17.72 | A |
| ATOM | 311 | N | LYS | A | 69 | 12.804 | 43.106 | 15.497 | 1.00 | 19.17 | A |
| ATOM | 312 | CA | LYS | A | 69 | 12.387 | 42.127 | 16.488 | 1.00 | 20.70 | A |
| ATOM | 313 | CB | LYS | A | 69 | 10.977 | 41.616 | 16.171 | 1.00 | 22.97 | A |
| ATOM | 314 | CG | LYS | A | 69 | 9.890 | 42.661 | 16.395 | 1.00 | 25.78 | A |
| ATOM | 315 | CD | LYS | A | 69 | 8.500 | 42.137 | 16.054 | 1.00 | 28.76 | A |
| ATOM | 316 | CE | LYS | A | 69 | 8.355 | 41.859 | 14.566 | 1.00 | 29.99 | A |
| ATOM | 317 | NZ | LYS | A | 69 | 6.952 | 41.507 | 14.203 | 1.00 | 31.16 | A |
| ATOM | 318 | C | LYS | A | 69 | 13.386 | 40.981 | 16.465 | 1.00 | 20.51 | A |
| ATOM | 319 | O | LYS | A | 69 | 13.944 | 40.659 | 15.416 | 1.00 | 20.04 | A |
| ATOM | 320 | N | TYR | A | 70 | 13.630 | 40.378 | 17.623 | 1.00 | 20.85 | A |
| ATOM | 321 | CA | TYR | A | 70 | 14.568 | 39.268 | 17.702 | 1.00 | 21.35 | A |
| ATOM | 322 | CB | TYR | A | 70 | 15.959 | 39.770 | 18.116 | 1.00 | 20.92 | A |
| ATOM | 323 | CG | TYR | A | 70 | 16.035 | 40.362 | 19.508 | 1.00 | 20.30 | A |
| ATOM | 324 | CD1 | TYR | A | 70 | 16.151 | 39.544 | 20.634 | 1.00 | 20.51 | A |
| ATOM | 325 | CE1 | TYR | A | 70 | 16.223 | 40.089 | 21.915 | 1.00 | 20.34 | A |
| ATOM | 326 | CD2 | TYR | A | 70 | 15.989 | 41.741 | 19.700 | 1.00 | 20.52 | A |
| ATOM | 327 | CE2 | TYR | A | 70 | 16.059 | 42.295 | 20.974 | 1.00 | 20.68 | A |
| ATOM | 328 | CZ | TYR | A | 70 | 16.175 | 41.466 | 22.076 | 1.00 | 20.53 | A |
| ATOM | 329 | OH | TYR | A | 70 | 16.238 | 42.018 | 23.334 | 1.00 | 21.78 | A |
| ATOM | 330 | C | TYR | A | 70 | 14.082 | 38.215 | 18.685 | 1.00 | 22.16 | A |
| ATOM | 331 | O | TYR | A | 70 | 13.295 | 38.506 | 19.587 | 1.00 | 22.38 | A |
| ATOM | 332 | N | ASP | A | 71 | 14.548 | 36.988 | 18.493 | 1.00 | 23.35 | A |
| ATOM | 333 | CA | ASP | A | 71 | 14.179 | 35.885 | 19.366 | 1.00 | 24.52 | A |
| ATOM | 334 | CB | ASP | A | 71 | 14.123 | 34.585 | 18.560 | 1.00 | 25.65 | A |
| ATOM | 335 | CG | ASP | A | 71 | 13.887 | 33.368 | 19.431 | 1.00 | 26.58 | A |
| ATOM | 336 | OD1 | ASP | A | 71 | 13.235 | 33.505 | 20.487 | 1.00 | 27.68 | A |
| ATOM | 337 | OD2 | ASP | A | 71 | 14.345 | 32.271 | 19.047 | 1.00 | 28.15 | A |
| ATOM | 338 | C | ASP | A | 71 | 15.219 | 35.792 | 20.477 | 1.00 | 25.08 | A |
| ATOM | 339 | O | ASP | A | 71 | 16.368 | 35.427 | 20.234 | 1.00 | 25.05 | A |
| ATOM | 340 | N | PRO | A | 72 | 14.825 | 36.126 | 21.716 | 1.00 | 25.93 | A |
| ATOM | 341 | CD | PRO | A | 72 | 13.445 | 36.362 | 22.173 | 1.00 | 25.99 | A |
| ATOM | 342 | CA | PRO | A | 72 | 15.746 | 36.077 | 22.855 | 1.00 | 26.65 | A |
| ATOM | 343 | CB | PRO | A | 72 | 14.839 | 36.385 | 24.048 | 1.00 | 26.60 | A |
| ATOM | 344 | CG | PRO | A | 72 | 13.502 | 35.879 | 23.600 | 1.00 | 26.93 | A |
| ATOM | 345 | C | PRO | A | 72 | 16.481 | 34.750 | 23.002 | 1.00 | 26.94 | A |
| ATOM | 346 | O | PRO | A | 72 | 17.587 | 34.701 | 23.540 | 1.00 | 27.06 | A |
| ATOM | 347 | N | LEU | A | 73 | 15.869 | 33.679 | 22.507 | 1.00 | 27.10 | A |
| ATOM | 348 | CA | LEU | A | 73 | 16.465 | 32.353 | 22.595 | 1.00 | 27.36 | A |
| ATOM | 349 | CB | LEU | A | 73 | 15.371 | 31.285 | 22.502 | 1.00 | 28.10 | A |
| ATOM | 350 | CG | LEU | A | 73 | 14.303 | 31.343 | 23.599 | 1.00 | 28.89 | A |
| ATOM | 351 | CD1 | LEU | A | 73 | 13.227 | 30.303 | 23.328 | 1.00 | 29.29 | A |
| ATOM | 352 | CD2 | LEU | A | 73 | 14.951 | 31.109 | 24.958 | 1.00 | 29.04 | A |
| ATOM | 353 | C | LEU | A | 73 | 17.522 | 32.105 | 21.521 | 1.00 | 27.02 | A |
| ATOM | 354 | O | LEU | A | 73 | 18.121 | 31.031 | 21.468 | 1.00 | 26.79 | A |
| ATOM | 355 | N | LYS | A | 74 | 17.756 | 33.099 | 20.669 | 1.00 | 26.77 | A |
| ATOM | 356 | CA | LYS | A | 74 | 18.748 | 32.960 | 19.611 | 1.00 | 27.29 | A |
| ATOM | 357 | CB | LYS | A | 74 | 18.743 | 34.193 | 18.707 | 1.00 | 28.07 | A |
| ATOM | 358 | CG | LYS | A | 74 | 19.729 | 34.113 | 17.553 | 1.00 | 29.63 | A |
| ATOM | 359 | CD | LYS | A | 74 | 19.556 | 35.284 | 16.603 | 1.00 | 30.36 | A |
| ATOM | 360 | CE | LYS | A | 74 | 20.482 | 35.162 | 15.405 | 1.00 | 31.13 | A |
| ATOM | 361 | NZ | LYS | A | 74 | 20.256 | 36.260 | 14.427 | 1.00 | 31.11 | A |
| ATOM | 362 | C | LYS | A | 74 | 20.141 | 32.762 | 20.200 | 1.00 | 27.08 | A |
| ATOM | 363 | O | LYS | A | 74 | 20.942 | 31.990 | 19.678 | 1.00 | 26.39 | A |
| ATOM | 364 | N | TYR | A | 75 | 20.428 | 33.470 | 21.286 | 1.00 | 27.82 | A |
| ATOM | 365 | CA | TYR | A | 75 | 21.724 | 33.347 | 21.936 | 1.00 | 28.44 | A |
| ATOM | 366 | CB | TYR | A | 75 | 22.359 | 34.730 | 22.130 | 1.00 | 28.35 | A |
| ATOM | 367 | CG | TYR | A | 75 | 22.677 | 35.420 | 20.821 | 1.00 | 28.28 | A |
| ATOM | 368 | CD1 | TYR | A | 75 | 21.796 | 36.346 | 20.261 | 1.00 | 28.89 | A |
| ATOM | 369 | CE1 | TYR | A | 75 | 22.058 | 36.931 | 19.021 | 1.00 | 28.65 | A |
| ATOM | 370 | CD2 | TYR | A | 75 | 23.832 | 35.098 | 20.111 | 1.00 | 28.33 | A |
| ATOM | 371 | CE2 | TYR | A | 75 | 24.101 | 35.673 | 18.872 | 1.00 | 29.04 | A |
| ATOM | 372 | CZ | TYR | A | 75 | 23.211 | 36.585 | 18.333 | 1.00 | 28.89 | A |
| ATOM | 373 | OH | TYR | A | 75 | 23.471 | 37.131 | 17.095 | 1.00 | 29.25 | A |
| ATOM | 374 | C | TYR | A | 75 | 21.577 | 32.634 | 23.274 | 1.00 | 29.07 | A |
| ATOM | 375 | O | TYR | A | 75 | 20.599 | 32.840 | 23.992 | 1.00 | 29.15 | A |
| ATOM | 376 | N | ASN | A | 76 | 22.547 | 31.782 | 23.591 | 1.00 | 29.40 | A |
| ATOM | 377 | CA | ASN | A | 76 | 22.533 | 31.029 | 24.839 | 1.00 | 30.49 | A |
| ATOM | 378 | CB | ASN | A | 76 | 21.742 | 29.729 | 24.668 | 1.00 | 31.72 | A |
| ATOM | 379 | CG | ASN | A | 76 | 22.463 | 28.717 | 23.804 | 1.00 | 32.78 | A |
| ATOM | 380 | OD1 | ASN | A | 76 | 22.765 | 28.979 | 22.643 | 1.00 | 33.93 | A |
| ATOM | 381 | ND2 | ASN | A | 76 | 22.746 | 27.550 | 24.372 | 1.00 | 34.66 | A |
| ATOM | 382 | C | ASN | A | 76 | 23.962 | 30.710 | 25.265 | 1.00 | 30.43 | A |
| ATOM | 383 | O | ASN | A | 76 | 24.919 | 31.143 | 24.626 | 1.00 | 29.75 | A |
| ATOM | 384 | N | ALA | A | 77 | 24.101 | 29.945 | 26.343 | 1.00 | 30.87 | A |
| ATOM | 385 | CA | ALA | A | 77 | 25.416 | 29.580 | 26.857 | 1.00 | 31.38 | A |
| ATOM | 386 | CB | ALA | A | 77 | 25.264 | 28.622 | 28.033 | 1.00 | 31.94 | A |
| ATOM | 387 | C | ALA | A | 77 | 26.316 | 28.957 | 25.794 | 1.00 | 31.58 | A |
| ATOM | 388 | O | ALA | A | 77 | 27.535 | 29.119 | 25.834 | 1.00 | 31.89 | A |
| ATOM | 389 | N | HIS | A | 78 | 25.715 | 28.252 | 24.841 | 1.00 | 31.69 | A |
| ATOM | 390 | CA | HIS | A | 78 | 26.481 | 27.599 | 23.785 | 1.00 | 31.63 | A |
| ATOM | 391 | CB | HIS | A | 78 | 25.811 | 26.278 | 23.399 | 1.00 | 33.87 | A |
| ATOM | 392 | CG | HIS | A | 78 | 25.580 | 25.359 | 24.557 | 1.00 | 35.97 | A |
| ATOM | 393 | CD2 | HIS | A | 78 | 24.448 | 24.784 | 25.028 | 1.00 | 36.95 | A |
| ATOM | 394 | ND1 | HIS | A | 78 | 26.597 | 24.938 | 25.388 | 1.00 | 37.02 | A |
| ATOM | 395 | CE1 | HIS | A | 78 | 26.101 | 24.144 | 26.320 | 1.00 | 37.60 | A |
| ATOM | 396 | NE2 | HIS | A | 78 | 24.799 | 24.034 | 26.124 | 1.00 | 37.80 | A |
| ATOM | 397 | C | HIS | A | 78 | 26.646 | 28.470 | 22.545 | 1.00 | 30.07 | A |
| ATOM | 398 | O | HIS | A | 78 | 27.360 | 28.103 | 21.612 | 1.00 | 30.09 | A |
| ATOM | 399 | N | HIS | A | 79 | 25.989 | 29.625 | 22.541 | 1.00 | 27.75 | A |
| ATOM | 400 | CA | HIS | A | 79 | 26.066 | 30.541 | 21.409 | 1.00 | 25.19 | A |
| ATOM | 401 | CB | HIS | A | 79 | 25.030 | 30.141 | 20.354 | 1.00 | 25.68 | A |
| ATOM | 402 | CG | HIS | A | 79 | 25.122 | 30.926 | 19.082 | 1.00 | 25.61 | A |
| ATOM | 403 | CD2 | HIS | A | 79 | 25.873 | 30.737 | 17.971 | 1.00 | 25.84 | A |
| ATOM | 404 | ND1 | HIS | A | 79 | 24.386 | 32.069 | 18.856 | 1.00 | 26.22 | A |
| ATOM | 405 | CE1 | HIS | A | 79 | 24.679 | 32.549 | 17.661 | 1.00 | 25.96 | A |
| ATOM | 406 | NE2 | HIS | A | 79 | 25.579 | 31.759 | 17.103 | 1.00 | 25.37 | A |
| ATOM | 407 | C | HIS | A | 79 | 25.822 | 31.965 | 21.897 | 1.00 | 22.98 | A |
| ATOM | 408 | O | HIS | A | 79 | 24.692 | 32.449 | 21.906 | 1.00 | 22.36 | A |
| ATOM | 409 | N | LYS | A | 80 | 26.899 | 32.626 | 22.307 | 1.00 | 20.90 | A |
| ATOM | 410 | CA | LYS | A | 80 | 26.821 | 33.985 | 22.825 | 1.00 | 19.06 | A |
| ATOM | 411 | CB | LYS | A | 80 | 27.850 | 34.187 | 23.937 | 1.00 | 20.08 | A |
| ATOM | 412 | CG | LYS | A | 80 | 27.757 | 33.211 | 25.095 | 1.00 | 21.73 | A |
| ATOM | 413 | CD | LYS | A | 80 | 28.851 | 33.513 | 26.106 | 1.00 | 23.84 | A |
| ATOM | 414 | CE | LYS | A | 80 | 28.813 | 32.556 | 27.283 | 1.00 | 25.02 | A |
| ATOM | 415 | NZ | LYS | A | 80 | 29.906 | 32.856 | 28.253 | 1.00 | 26.36 | A |
| ATOM | 416 | C | LYS | A | 80 | 27.071 | 35.042 | 21.761 | 1.00 | 17.49 | A |
| ATOM | 417 | O | LYS | A | 80 | 27.679 | 34.772 | 20.726 | 1.00 | 17.57 | A |
| ATOM | 418 | N | LEU | A | 81 | 26.596 | 36.251 | 22.035 | 1.00 | 14.89 | A |
| ATOM | 419 | CA | LEU | A | 81 | 26.796 | 37.376 | 21.134 | 1.00 | 13.66 | A |
| ATOM | 420 | CB | LEU | A | 81 | 25.622 | 38.352 | 21.223 | 1.00 | 13.09 | A |
| ATOM | 421 | CG | LEU | A | 81 | 25.728 | 39.609 | 20.349 | 1.00 | 12.56 | A |
| ATOM | 422 | CD1 | LEU | A | 81 | 25.752 | 39.205 | 18.874 | 1.00 | 13.02 | A |
| ATOM | 423 | CD2 | LEU | A | 81 | 24.553 | 40.541 | 20.631 | 1.00 | 14.10 | A |
| ATOM | 424 | C | LEU | A | 81 | 28.075 | 38.067 | 21.594 | 1.00 | 14.00 | A |
| ATOM | 425 | O | LEU | A | 81 | 28.161 | 38.525 | 22.733 | 1.00 | 13.98 | A |
| ATOM | 426 | N | LYS | A | 82 | 29.070 | 38.121 | 20.714 | 1.00 | 12.74 | A |
| ATOM | 427 | CA | LYS | A | 82 | 30.344 | 38.759 | 21.028 | 1.00 | 13.21 | A |
| ATOM | 428 | CB | LYS | A | 82 | 31.487 | 38.017 | 20.328 | 1.00 | 15.38 | A |
| ATOM | 429 | CG | LYS | A | 82 | 31.631 | 36.570 | 20.782 | 1.00 | 19.55 | A |
| ATOM | 430 | CD | LYS | A | 82 | 32.517 | 35.748 | 19.852 | 1.00 | 22.61 | A |
| ATOM | 431 | CE | LYS | A | 82 | 33.960 | 36.225 | 19.859 | 1.00 | 24.15 | A |
| ATOM | 432 | NZ | LYS | A | 82 | 34.815 | 35.366 | 18.989 | 1.00 | 26.27 | A |
| ATOM | 433 | C | LYS | A | 82 | 30.253 | 40.191 | 20.533 | 1.00 | 12.63 | A |
| ATOM | 434 | O | LYS | A | 82 | 30.047 | 40.427 | 19.343 | 1.00 | 13.50 | A |
| ATOM | 435 | N | VAL | A | 83 | 30.399 | 41.142 | 21.451 | 1.00 | 11.19 | A |
| ATOM | 436 | CA | VAL | A | 83 | 30.296 | 42.553 | 21.112 | 1.00 | 11.54 | A |
| ATOM | 437 | CB | VAL | A | 83 | 29.237 | 43.245 | 22.000 | 1.00 | 10.36 | A |
| ATOM | 438 | CG1 | VAL | A | 83 | 29.078 | 44.708 | 21.601 | 1.00 | 10.99 | A |
| ATOM | 439 | CG2 | VAL | A | 83 | 27.911 | 42.516 | 21.873 | 1.00 | 11.60 | A |
| ATOM | 440 | C | VAL | A | 83 | 31.613 | 43.300 | 21.260 | 1.00 | 11.60 | A |
| ATOM | 441 | O | VAL | A | 83 | 32.242 | 43.278 | 22.318 | 1.00 | 11.88 | A |
| ATOM | 442 | N | PHE | A | 84 | 32.023 | 43.969 | 20.187 | 1.00 | 10.64 | A |
| ATOM | 443 | CA | PHE | A | 84 | 33.247 | 44.753 | 20.206 | 1.00 | 11.09 | A |
| ATOM | 444 | CB | PHE | A | 84 | 34.150 | 44.394 | 19.025 | 1.00 | 12.04 | A |
| ATOM | 445 | CG | PHE | A | 84 | 34.799 | 43.048 | 19.144 | 1.00 | 12.77 | A |
| ATOM | 446 | CD1 | PHE | A | 84 | 34.299 | 41.954 | 18.450 | 1.00 | 13.33 | A |
| ATOM | 447 | CD2 | PHE | A | 84 | 35.915 | 42.876 | 19.955 | 1.00 | 14.39 | A |
| ATOM | 448 | CE1 | PHE | A | 84 | 34.903 | 40.702 | 18.561 | 1.00 | 15.01 | A |
| ATOM | 449 | CE2 | PHE | A | 84 | 36.528 | 41.632 | 20.076 | 1.00 | 14.37 | A |
| ATOM | 450 | CZ | PHE | A | 84 | 36.020 | 40.542 | 19.375 | 1.00 | 14.86 | A |
| ATOM | 451 | C | PHE | A | 84 | 32.901 | 46.234 | 20.135 | 1.00 | 10.24 | A |
| ATOM | 452 | O | PHE | A | 84 | 32.378 | 46.706 | 19.125 | 1.00 | 10.64 | A |
| ATOM | 453 | N | VAL | A | 85 | 33.172 | 46.952 | 21.222 | 1.00 | 9.19 | A |
| ATOM | 454 | CA | VAL | A | 85 | 32.933 | 48.389 | 21.287 | 1.00 | 9.61 | A |
| ATOM | 455 | CB | VAL | A | 85 | 32.563 | 48.823 | 22.718 | 1.00 | 8.87 | A |
| ATOM | 456 | CG1 | VAL | A | 85 | 32.403 | 50.334 | 22.787 | 1.00 | 10.61 | A |
| ATOM | 457 | CG2 | VAL | A | 85 | 31.269 | 48.132 | 23.138 | 1.00 | 10.27 | A |
| ATOM | 458 | C | VAL | A | 85 | 34.258 | 49.012 | 20.865 | 1.00 | 8.92 | A |
| ATOM | 459 | O | VAL | A | 85 | 35.274 | 48.855 | 21.546 | 1.00 | 9.82 | A |
| ATOM | 460 | N | VAL | A | 86 | 34.236 | 49.716 | 19.735 | 1.00 | 7.99 | A |
| ATOM | 461 | CA | VAL | A | 86 | 35.438 | 50.310 | 19.161 | 1.00 | 8.62 | A |
| ATOM | 462 | CB | VAL | A | 86 | 35.561 | 49.893 | 17.675 | 1.00 | 8.93 | A |
| ATOM | 463 | CG1 | VAL | A | 86 | 36.882 | 50.390 | 17.093 | 1.00 | 9.60 | A |
| ATOM | 464 | CG2 | VAL | A | 86 | 35.458 | 48.373 | 17.557 | 1.00 | 10.05 | A |
| ATOM | 465 | C | VAL | A | 86 | 35.499 | 51.833 | 19.267 | 1.00 | 7.68 | A |
| ATOM | 466 | O | VAL | A | 86 | 34.862 | 52.551 | 18.489 | 1.00 | 7.87 | A |
| ATOM | 467 | N | PRO | A | 87 | 36.282 | 52.348 | 20.230 | 1.00 | 8.07 | A |
| ATOM | 468 | CD | PRO | A | 87 | 36.951 | 51.612 | 21.316 | 1.00 | 8.47 | A |
| ATOM | 469 | CA | PRO | A | 87 | 36.420 | 53.795 | 20.423 | 1.00 | 8.11 | A |
| ATOM | 470 | CB | PRO | A | 87 | 37.274 | 53.896 | 21.690 | 1.00 | 8.31 | A |
| ATOM | 471 | CG | PRO | A | 87 | 36.939 | 52.630 | 22.428 | 1.00 | 9.52 | A |
| ATOM | 472 | C | PRO | A | 87 | 37.109 | 54.437 | 19.222 | 1.00 | 8.18 | A |
| ATOM | 473 | O | PRO | A | 87 | 38.100 | 53.906 | 18.716 | 1.00 | 7.38 | A |
| ATOM | 474 | N | HIS | A | 88 | 36.580 | 55.568 | 18.766 | 1.00 | 7.67 | A |
| ATOM | 475 | CA | HIS | A | 88 | 37.169 | 56.267 | 17.630 | 1.00 | 8.52 | A |
| ATOM | 476 | CB | HIS | A | 88 | 36.613 | 55.709 | 16.308 | 1.00 | 8.65 | A |
| ATOM | 477 | CG | HIS | A | 88 | 35.167 | 56.015 | 16.077 | 1.00 | 9.36 | A |
| ATOM | 478 | CD2 | HIS | A | 88 | 34.045 | 55.391 | 16.505 | 1.00 | 9.20 | A |
| ATOM | 479 | ND1 | HIS | A | 88 | 34.744 | 57.098 | 15.335 | 1.00 | 8.08 | A |
| ATOM | 480 | CE1 | HIS | A | 88 | 33.423 | 57.126 | 15.317 | 1.00 | 8.99 | A |
| ATOM | 481 | NE2 | HIS | A | 88 | 32.974 | 56.102 | 16.021 | 1.00 | 9.70 | A |
| ATOM | 482 | C | HIS | A | 88 | 36.927 | 57.765 | 17.718 | 1.00 | 9.68 | A |
| ATOM | 483 | O | HIS | A | 88 | 36.108 | 58.238 | 18.512 | 1.00 | 8.71 | A |
| ATOM | 484 | N | SER | A | 89 | 37.661 | 58.511 | 16.904 | 1.00 | 8.64 | A |
| ATOM | 485 | CA | SER | A | 89 | 37.551 | 59.958 | 16.889 | 1.00 | 8.97 | A |
| ATOM | 486 | CB | SER | A | 89 | 38.657 | 60.562 | 17.758 | 1.00 | 9.29 | A |
| ATOM | 487 | OG | SER | A | 89 | 38.626 | 61.978 | 17.733 | 1.00 | 9.37 | A |
| ATOM | 488 | C | SER | A | 89 | 37.708 | 60.408 | 15.449 | 1.00 | 8.81 | A |
| ATOM | 489 | O | SER | A | 89 | 38.771 | 60.233 | 14.856 | 1.00 | 8.86 | A |
| ATOM | 490 | N | HIS | A | 90 | 36.648 | 60.971 | 14.881 | 1.00 | 8.95 | A |
| ATOM | 491 | CA | HIS | A | 90 | 36.714 | 61.427 | 13.499 | 1.00 | 8.92 | A |
| ATOM | 492 | CB | HIS | A | 90 | 35.313 | 61.494 | 12.895 | 1.00 | 9.62 | A |
| ATOM | 493 | CG | HIS | A | 90 | 35.310 | 61.809 | 11.434 | 1.00 | 8.56 | A |
| ATOM | 494 | CD2 | HIS | A | 90 | 34.836 | 62.880 | 10.757 | 1.00 | 9.62 | A |
| ATOM | 495 | ND1 | HIS | A | 90 | 35.874 | 60.977 | 10.491 | 1.00 | 9.09 | A |
| ATOM | 496 | CE1 | HIS | A | 90 | 35.748 | 61.523 | 9.295 | 1.00 | 9.31 | A |
| ATOM | 497 | NE2 | HIS | A | 90 | 35.122 | 62.679 | 9.430 | 1.00 | 8.59 | A |
| ATOM | 498 | C | HIS | A | 90 | 37.391 | 62.792 | 13.418 | 1.00 | 8.62 | A |
| ATOM | 499 | O | HIS | A | 90 | 36.849 | 63.799 | 13.883 | 1.00 | 9.74 | A |
| ATOM | 500 | N | ASN | A | 91 | 38.584 | 62.817 | 12.829 | 1.00 | 9.20 | A |
| ATOM | 501 | CA | ASN | A | 91 | 39.354 | 64.052 | 12.706 | 1.00 | 9.16 | A |
| ATOM | 502 | CB | ASN | A | 91 | 40.744 | 63.867 | 13.317 | 1.00 | 9.59 | A |
| ATOM | 503 | CG | ASN | A | 91 | 40.703 | 63.716 | 14.822 | 1.00 | 9.87 | A |
| ATOM | 504 | OD1 | ASN | A | 91 | 40.092 | 62.787 | 15.351 | 1.00 | 12.09 | A |
| ATOM | 505 | ND2 | ASN | A | 91 | 41.353 | 64.633 | 15.521 | 1.00 | 7.48 | A |
| ATOM | 506 | C | ASN | A | 91 | 39.504 | 64.516 | 11.266 | 1.00 | 10.50 | A |
| ATOM | 507 | O | ASN | A | 91 | 40.300 | 63.969 | 10.503 | 1.00 | 11.67 | A |
| ATOM | 508 | N | ASP | A | 92 | 38.738 | 65.534 | 10.900 | 1.00 | 9.47 | A |
| ATOM | 509 | CA | ASP | A | 92 | 38.796 | 66.078 | 9.551 | 1.00 | 9.42 | A |
| ATOM | 510 | CB | ASP | A | 92 | 37.562 | 66.934 | 9.282 | 1.00 | 9.14 | A |
| ATOM | 511 | CG | ASP | A | 92 | 36.314 | 66.113 | 9.149 | 1.00 | 10.48 | A |
| ATOM | 512 | OD1 | ASP | A | 92 | 36.328 | 65.197 | 8.310 | 1.00 | 10.31 | A |
| ATOM | 513 | OD2 | ASP | A | 92 | 35.328 | 66.372 | 9.873 | 1.00 | 12.87 | A |
| ATOM | 514 | C | ASP | A | 92 | 40.034 | 66.930 | 9.337 | 1.00 | 9.30 | A |
| ATOM | 515 | O | ASP | A | 92 | 40.256 | 67.890 | 10.067 | 1.00 | 9.36 | A |
| ATOM | 516 | N | PRO | A | 93 | 40.864 | 66.582 | 8.338 | 1.00 | 9.43 | A |
| ATOM | 517 | CD | PRO | A | 93 | 40.895 | 65.288 | 7.638 | 1.00 | 8.82 | A |
| ATOM | 518 | CA | PRO | A | 93 | 42.078 | 67.351 | 8.048 | 1.00 | 9.47 | A |
| ATOM | 519 | CB | PRO | A | 93 | 42.877 | 66.416 | 7.140 | 1.00 | 9.10 | A |
| ATOM | 520 | CG | PRO | A | 93 | 42.378 | 65.052 | 7.507 | 1.00 | 11.35 | A |
| ATOM | 521 | C | PRO | A | 93 | 41.655 | 68.632 | 7.336 | 1.00 | 10.26 | A |
| ATOM | 522 | O | PRO | A | 93 | 42.020 | 68.883 | 6.182 | 1.00 | 10.84 | A |
| ATOM | 523 | N | GLY | A | 94 | 40.859 | 69.424 | 8.048 | 1.00 | 9.66 | A |
| ATOM | 524 | CA | GLY | A | 94 | 40.336 | 70.663 | 7.516 | 1.00 | 10.36 | A |
| ATOM | 525 | C | GLY | A | 94 | 38.862 | 70.516 | 7.177 | 1.00 | 10.89 | A |
| ATOM | 526 | O | GLY | A | 94 | 38.440 | 69.492 | 6.634 | 1.00 | 10.38 | A |
| ATOM | 527 | N | TRP | A | 95 | 38.082 | 71.528 | 7.538 | 1.00 | 10.58 | A |
| ATOM | 528 | CA | TRP | A | 95 | 36.653 | 71.588 | 7.245 | 1.00 | 10.62 | A |
| ATOM | 529 | CB | TRP | A | 95 | 35.854 | 70.479 | 7.948 | 1.00 | 10.51 | A |
| ATOM | 530 | CG | TRP | A | 95 | 34.387 | 70.607 | 7.634 | 1.00 | 11.04 | A |
| ATOM | 531 | CD2 | TRP | A | 95 | 33.288 | 70.466 | 8.545 | 1.00 | 11.08 | A |
| ATOM | 532 | CE2 | TRP | A | 95 | 32.109 | 70.765 | 7.825 | 1.00 | 11.49 | A |
| ATOM | 533 | CE3 | TRP | A | 95 | 33.184 | 70.120 | 9.901 | 1.00 | 11.93 | A |
| ATOM | 534 | CD1 | TRP | A | 95 | 33.840 | 70.963 | 6.431 | 1.00 | 10.57 | A |
| ATOM | 535 | NE1 | TRP | A | 95 | 32.475 | 71.065 | 6.539 | 1.00 | 10.89 | A |
| ATOM | 536 | CZ2 | TRP | A | 95 | 30.839 | 70.731 | 8.415 | 1.00 | 12.07 | A |
| ATOM | 537 | CZ3 | TRP | A | 95 | 31.918 | 70.086 | 10.487 | 1.00 | 12.97 | A |
| ATOM | 538 | CH2 | TRP | A | 95 | 30.765 | 70.391 | 9.741 | 1.00 | 13.12 | A |
| ATOM | 539 | C | TRP | A | 95 | 36.151 | 72.968 | 7.669 | 1.00 | 11.53 | A |
| ATOM | 540 | O | TRP | A | 95 | 36.063 | 73.865 | 6.834 | 1.00 | 10.86 | A |
| ATOM | 541 | N | ILE | A | 96 | 35.829 | 73.151 | 8.947 | 1.00 | 11.91 | A |
| ATOM | 542 | CA | ILE | A | 96 | 35.389 | 74.467 | 9.405 | 1.00 | 13.09 | A |
| ATOM | 543 | CB | ILE | A | 96 | 34.240 | 74.389 | 10.434 | 1.00 | 14.40 | A |
| ATOM | 544 | CG2 | ILE | A | 96 | 32.993 | 73.840 | 9.758 | 1.00 | 15.73 | A |
| ATOM | 545 | CG1 | ILE | A | 96 | 34.656 | 73.549 | 11.638 | 1.00 | 16.78 | A |
| ATOM | 546 | CD1 | ILE | A | 96 | 33.689 | 73.638 | 12.798 | 1.00 | 18.96 | A |
| ATOM | 547 | C | ILE | A | 96 | 36.579 | 75.207 | 10.007 | 1.00 | 12.25 | A |
| ATOM | 548 | O | ILE | A | 96 | 36.486 | 76.378 | 10.374 | 1.00 | 13.34 | A |
| ATOM | 549 | N | GLN | A | 97 | 37.698 | 74.496 | 10.102 | 1.00 | 11.48 | A |
| ATOM | 550 | CA | GLN | A | 97 | 38.960 | 75.042 | 10.585 | 1.00 | 10.81 | A |
| ATOM | 551 | CB | GLN | A | 97 | 39.239 | 74.617 | 12.030 | 1.00 | 13.00 | A |
| ATOM | 552 | CG | GLN | A | 97 | 38.316 | 75.252 | 13.059 | 1.00 | 15.72 | A |
| ATOM | 553 | CD | GLN | A | 97 | 38.781 | 75.011 | 14.481 | 1.00 | 18.59 | A |
| ATOM | 554 | OE1 | GLN | A | 97 | 39.922 | 75.320 | 14.834 | 1.00 | 21.45 | A |
| ATOM | 555 | NE2 | GLN | A | 97 | 37.899 | 74.460 | 15.309 | 1.00 | 20.61 | A |
| ATOM | 556 | C | GLN | A | 97 | 40.007 | 74.431 | 9.660 | 1.00 | 9.61 | A |
| ATOM | 557 | O | GLN | A | 97 | 39.740 | 73.424 | 9.007 | 1.00 | 9.16 | A |
| ATOM | 558 | N | THR | A | 98 | 41.185 | 75.037 | 9.580 | 1.00 | 9.03 | A |
| ATOM | 559 | CA | THR | A | 98 | 42.238 | 74.487 | 8.732 | 1.00 | 8.85 | A |
| ATOM | 560 | CB | THR | A | 98 | 43.363 | 75.495 | 8.486 | 1.00 | 9.21 | A |
| ATOM | 561 | OG1 | THR | A | 98 | 43.987 | 75.813 | 9.736 | 1.00 | 9.92 | A |
| ATOM | 562 | CG2 | THR | A | 98 | 42.818 | 76.769 | 7.854 | 1.00 | 8.60 | A |
| ATOM | 563 | C | THR | A | 98 | 42.862 | 73.289 | 9.437 | 1.00 | 8.61 | A |
| ATOM | 564 | O | THR | A | 98 | 42.598 | 73.039 | 10.617 | 1.00 | 8.91 | A |
| ATOM | 565 | N | PHE | A | 99 | 43.686 | 72.552 | 8.704 | 1.00 | 8.51 | A |
| ATOM | 566 | CA | PHE | A | 99 | 44.377 | 71.395 | 9.255 | 1.00 | 8.31 | A |
| ATOM | 567 | CB | PHE | A | 99 | 45.359 | 70.837 | 8.220 | 1.00 | 8.62 | A |
| ATOM | 568 | CG | PHE | A | 99 | 46.236 | 69.737 | 8.745 | 1.00 | 8.46 | A |
| ATOM | 569 | CD1 | PHE | A | 99 | 45.831 | 68.407 | 8.668 | 1.00 | 8.79 | A |
| ATOM | 570 | CD2 | PHE | A | 99 | 47.469 | 70.031 | 9.322 | 1.00 | 9.06 | A |
| ATOM | 571 | CE1 | PHE | A | 99 | 46.642 | 67.383 | 9.156 | 1.00 | 9.28 | A |
| ATOM | 572 | CE2 | PHE | A | 99 | 48.286 | 69.020 | 9.813 | 1.00 | 9.90 | A |
| ATOM | 573 | CZ | PHE | A | 99 | 47.873 | 67.687 | 9.730 | 1.00 | 8.95 | A |
| ATOM | 574 | C | PHE | A | 99 | 45.144 | 71.809 | 10.509 | 1.00 | 8.94 | A |
| ATOM | 575 | O | PHE | A | 99 | 45.011 | 71.193 | 11.566 | 1.00 | 8.71 | A |
| ATOM | 576 | N | GLU | A | 100 | 45.948 | 72.861 | 10.386 | 1.00 | 9.46 | A |
| ATOM | 577 | CA | GLU | A | 100 | 46.756 | 73.331 | 11.505 | 1.00 | 10.17 | A |
| ATOM | 578 | CB | GLU | A | 100 | 47.739 | 74.405 | 11.026 | 1.00 | 10.59 | A |
| ATOM | 579 | CG | GLU | A | 100 | 48.778 | 74.836 | 12.059 | 1.00 | 12.88 | A |
| ATOM | 580 | CD | GLU | A | 100 | 49.649 | 73.692 | 12.552 | 1.00 | 14.60 | A |
| ATOM | 581 | OE1 | GLU | A | 100 | 49.825 | 72.698 | 11.812 | 1.00 | 14.74 | A |
| ATOM | 582 | OE2 | GLU | A | 100 | 50.177 | 73.797 | 13.680 | 1.00 | 15.45 | A |
| ATOM | 583 | C | GLU | A | 100 | 45.921 | 73.854 | 12.668 | 1.00 | 10.39 | A |
| ATOM | 584 | O | GLU | A | 100 | 46.275 | 73.630 | 13.828 | 1.00 | 9.60 | A |
| ATOM | 585 | N | GLU | A | 101 | 44.816 | 74.537 | 12.369 | 1.00 | 10.05 | A |
| ATOM | 586 | CA | GLU | A | 101 | 43.952 | 75.059 | 13.429 | 1.00 | 10.07 | A |
| ATOM | 587 | CB | GLU | A | 101 | 42.822 | 75.918 | 12.845 | 1.00 | 10.88 | A |
| ATOM | 588 | CG | GLU | A | 101 | 43.287 | 77.260 | 12.266 | 1.00 | 13.03 | A |
| ATOM | 589 | CD | GLU | A | 101 | 42.154 | 78.075 | 11.658 | 1.00 | 14.64 | A |
| ATOM | 590 | OE1 | GLU | A | 101 | 41.250 | 77.481 | 11.036 | 1.00 | 13.48 | A |
| ATOM | 591 | OE2 | GLU | A | 101 | 42.176 | 79.319 | 11.788 | 1.00 | 17.65 | A |
| ATOM | 592 | C | GLU | A | 101 | 43.366 | 73.901 | 14.234 | 1.00 | 9.98 | A |
| ATOM | 593 | O | GLU | A | 101 | 43.383 | 73.920 | 15.468 | 1.00 | 9.79 | A |
| ATOM | 594 | N | TYR | A | 102 | 42.846 | 72.892 | 13.539 | 1.00 | 9.17 | A |
| ATOM | 595 | CA | TYR | A | 102 | 42.286 | 71.726 | 14.222 | 1.00 | 9.45 | A |
| ATOM | 596 | CB | TYR | A | 102 | 41.704 | 70.719 | 13.231 | 1.00 | 9.69 | A |
| ATOM | 597 | CG | TYR | A | 102 | 40.295 | 70.970 | 12.749 | 1.00 | 9.66 | A |
| ATOM | 598 | CD1 | TYR | A | 102 | 39.247 | 71.205 | 13.643 | 1.00 | 10.46 | A |
| ATOM | 599 | CE1 | TYR | A | 102 | 37.928 | 71.319 | 13.188 | 1.00 | 9.87 | A |
| ATOM | 600 | CD2 | TYR | A | 102 | 39.989 | 70.865 | 11.392 | 1.00 | 9.78 | A |
| ATOM | 601 | CE2 | TYR | A | 102 | 38.688 | 70.973 | 10.934 | 1.00 | 10.12 | A |
| ATOM | 602 | CZ | TYR | A | 102 | 37.661 | 71.197 | 11.830 | 1.00 | 10.79 | A |
| ATOM | 603 | OH | TYR | A | 102 | 36.374 | 71.266 | 11.352 | 1.00 | 10.63 | A |
| ATOM | 604 | C | TYR | A | 102 | 43.375 | 71.009 | 15.008 | 1.00 | 10.28 | A |
| ATOM | 605 | O | TYR | A | 102 | 43.138 | 70.515 | 16.112 | 1.00 | 9.91 | A |
| ATOM | 606 | N | TYR | A | 103 | 44.567 | 70.926 | 14.429 | 1.00 | 10.93 | A |
| ATOM | 607 | CA | TYR | A | 103 | 45.656 | 70.245 | 15.108 | 1.00 | 11.21 | A |
| ATOM | 608 | CB | TYR | A | 103 | 46.920 | 70.226 | 14.250 | 1.00 | 11.30 | A |
| ATOM | 609 | CG | TYR | A | 103 | 48.077 | 69.577 | 14.968 | 1.00 | 11.06 | A |
| ATOM | 610 | CD1 | TYR | A | 103 | 48.080 | 68.207 | 15.224 | 1.00 | 11.12 | A |
| ATOM | 611 | CE1 | TYR | A | 103 | 49.103 | 67.614 | 15.954 | 1.00 | 10.89 | A |
| ATOM | 612 | CD2 | TYR | A | 103 | 49.137 | 70.342 | 15.460 | 1.00 | 11.87 | A |
| ATOM | 613 | CE2 | TYR | A | 103 | 50.164 | 69.760 | 16.195 | 1.00 | 11.51 | A |
| ATOM | 614 | CZ | TYR | A | 103 | 50.141 | 68.397 | 16.440 | 1.00 | 11.95 | A |
| ATOM | 615 | OH | TYR | A | 103 | 51.145 | 67.818 | 17.187 | 1.00 | 13.54 | A |
| ATOM | 616 | C | TYR | A | 103 | 45.971 | 70.909 | 16.440 | 1.00 | 11.40 | A |
| ATOM | 617 | O | TYR | A | 103 | 46.092 | 70.240 | 17.462 | 1.00 | 11.11 | A |
| ATOM | 618 | N | GLN | A | 104 | 46.099 | 72.231 | 16.422 | 1.00 | 11.78 | A |
| ATOM | 619 | CA | GLN | A | 104 | 46.419 | 72.981 | 17.631 | 1.00 | 13.08 | A |
| ATOM | 620 | CB | GLN | A | 104 | 46.770 | 74.427 | 17.271 | 1.00 | 12.77 | A |
| ATOM | 621 | CG | GLN | A | 104 | 48.091 | 74.597 | 16.541 | 1.00 | 13.19 | A |
| ATOM | 622 | CD | GLN | A | 104 | 49.268 | 74.058 | 17.336 | 1.00 | 14.03 | A |
| ATOM | 623 | OE1 | GLN | A | 104 | 49.305 | 74.172 | 18.564 | 1.00 | 14.60 | A |
| ATOM | 624 | NE2 | GLN | A | 104 | 50.242 | 73.483 | 16.640 | 1.00 | 14.23 | A |
| ATOM | 625 | C | GLN | A | 104 | 45.301 | 72.992 | 18.667 | 1.00 | 14.02 | A |
| ATOM | 626 | O | GLN | A | 104 | 45.552 | 72.849 | 19.863 | 1.00 | 14.69 | A |
| ATOM | 627 | N | HIS | A | 105 | 44.067 | 73.152 | 18.202 | 1.00 | 14.24 | A |
| ATOM | 628 | CA | HIS | A | 105 | 42.912 | 73.235 | 19.091 | 1.00 | 15.29 | A |
| ATOM | 629 | CB | HIS | A | 105 | 41.796 | 74.035 | 18.412 | 1.00 | 18.09 | A |
| ATOM | 630 | CG | HIS | A | 105 | 42.228 | 75.377 | 17.907 | 1.00 | 21.36 | A |
| ATOM | 631 | CD2 | HIS | A | 105 | 43.322 | 76.126 | 18.181 | 1.00 | 23.58 | A |
| ATOM | 632 | ND1 | HIS | A | 105 | 41.481 | 76.105 | 17.005 | 1.00 | 23.59 | A |
| ATOM | 633 | CE1 | HIS | A | 105 | 42.098 | 77.244 | 16.744 | 1.00 | 24.20 | A |
| ATOM | 634 | NE2 | HIS | A | 105 | 43.217 | 77.281 | 17.445 | 1.00 | 24.69 | A |
| ATOM | 635 | C | HIS | A | 105 | 42.330 | 71.905 | 19.552 | 1.00 | 15.14 | A |
| ATOM | 636 | O | HIS | A | 105 | 41.815 | 71.807 | 20.665 | 1.00 | 15.70 | A |
| ATOM | 637 | N | ASP | A | 106 | 42.416 | 70.880 | 18.712 | 1.00 | 13.68 | A |
| ATOM | 638 | CA | ASP | A | 106 | 41.818 | 69.600 | 19.064 | 1.00 | 13.41 | A |
| ATOM | 639 | CB | ASP | A | 106 | 40.556 | 69.386 | 18.221 | 1.00 | 15.33 | A |
| ATOM | 640 | CG | ASP | A | 106 | 39.513 | 70.461 | 18.449 | 1.00 | 17.76 | A |
| ATOM | 641 | OD1 | ASP | A | 106 | 38.857 | 70.435 | 19.509 | 1.00 | 18.23 | A |
| ATOM | 642 | OD2 | ASP | A | 106 | 39.359 | 71.337 | 17.570 | 1.00 | 19.51 | A |
| ATOM | 643 | C | ASP | A | 106 | 42.673 | 68.346 | 18.960 | 1.00 | 12.48 | A |
| ATOM | 644 | O | ASP | A | 106 | 42.942 | 67.684 | 19.957 | 1.00 | 11.86 | A |
| ATOM | 645 | N | THR | A | 107 | 43.095 | 68.025 | 17.745 | 1.00 | 11.48 | A |
| ATOM | 646 | CA | THR | A | 107 | 43.845 | 66.805 | 17.490 | 1.00 | 10.31 | A |
| ATOM | 647 | CB | THR | A | 107 | 44.169 | 66.694 | 15.991 | 1.00 | 9.81 | A |
| ATOM | 648 | OG1 | THR | A | 107 | 42.964 | 66.919 | 15.247 | 1.00 | 9.72 | A |
| ATOM | 649 | CG2 | THR | A | 107 | 44.710 | 65.302 | 15.656 | 1.00 | 9.56 | A |
| ATOM | 650 | C | THR | A | 107 | 45.100 | 66.517 | 18.305 | 1.00 | 10.15 | A |
| ATOM | 651 | O | THR | A | 107 | 45.309 | 65.377 | 18.722 | 1.00 | 9.55 | A |
| ATOM | 652 | N | LYS | A | 108 | 45.940 | 67.515 | 18.555 | 1.00 | 10.06 | A |
| ATOM | 653 | CA | LYS | A | 108 | 47.142 | 67.223 | 19.323 | 1.00 | 10.48 | A |
| ATOM | 654 | CB | LYS | A | 108 | 48.109 | 68.416 | 19.322 | 1.00 | 11.26 | A |
| ATOM | 655 | CG | LYS | A | 108 | 47.753 | 69.597 | 20.206 | 1.00 | 12.62 | A |
| ATOM | 656 | CD | LYS | A | 108 | 48.842 | 70.661 | 20.066 | 1.00 | 13.56 | A |
| ATOM | 657 | CE | LYS | A | 108 | 48.632 | 71.837 | 21.000 | 1.00 | 14.90 | A |
| ATOM | 658 | NZ | LYS | A | 108 | 49.762 | 72.815 | 20.901 | 1.00 | 16.47 | A |
| ATOM | 659 | C | LYS | A | 108 | 46.777 | 66.809 | 20.744 | 1.00 | 10.06 | A |
| ATOM | 660 | O | LYS | A | 108 | 47.483 | 66.013 | 21.364 | 1.00 | 10.31 | A |
| ATOM | 661 | N | HIS | A | 109 | 45.663 | 67.330 | 21.246 | 1.00 | 10.31 | A |
| ATOM | 662 | CA | HIS | A | 109 | 45.210 | 66.985 | 22.590 | 1.00 | 10.51 | A |
| ATOM | 663 | CB | HIS | A | 109 | 44.215 | 68.031 | 23.086 | 1.00 | 12.57 | A |
| ATOM | 664 | CG | HIS | A | 109 | 44.791 | 69.410 | 23.154 | 1.00 | 14.27 | A |
| ATOM | 665 | CD2 | HIS | A | 109 | 44.510 | 70.530 | 22.448 | 1.00 | 16.35 | A |
| ATOM | 666 | ND1 | HIS | A | 109 | 45.821 | 69.743 | 24.008 | 1.00 | 16.64 | A |
| ATOM | 667 | CE1 | HIS | A | 109 | 46.148 | 71.010 | 23.825 | 1.00 | 16.26 | A |
| ATOM | 668 | NE2 | HIS | A | 109 | 45.368 | 71.510 | 22.884 | 1.00 | 16.75 | A |
| ATOM | 669 | C | HIS | A | 109 | 44.578 | 65.598 | 22.594 | 1.00 | 10.63 | A |
| ATOM | 670 | O | HIS | A | 109 | 44.765 | 64.824 | 23.530 | 1.00 | 10.59 | A |
| ATOM | 671 | N | ILE | A | 110 | 43.832 | 65.283 | 21.543 | 1.00 | 10.70 | A |
| ATOM | 672 | CA | ILE | A | 110 | 43.202 | 63.975 | 21.426 | 1.00 | 10.48 | A |
| ATOM | 673 | CB | ILE | A | 110 | 42.369 | 63.881 | 20.125 | 1.00 | 10.18 | A |
| ATOM | 674 | CG2 | ILE | A | 110 | 41.950 | 62.435 | 19.864 | 1.00 | 10.02 | A |
| ATOM | 675 | CG1 | ILE | A | 110 | 41.151 | 64.802 | 20.229 | 1.00 | 10.13 | A |
| ATOM | 676 | CD1 | ILE | A | 110 | 40.395 | 64.989 | 18.921 | 1.00 | 10.58 | A |
| ATOM | 677 | C | ILE | A | 110 | 44.279 | 62.889 | 21.407 | 1.00 | 10.11 | A |
| ATOM | 678 | O | ILE | A | 110 | 44.187 | 61.892 | 22.125 | 1.00 | 9.62 | A |
| ATOM | 679 | N | LEU | A | 111 | 45.307 | 63.087 | 20.591 | 1.00 | 10.21 | A |
| ATOM | 680 | CA | LEU | A | 111 | 46.382 | 62.112 | 20.490 | 1.00 | 10.24 | A |
| ATOM | 681 | CB | LEU | A | 111 | 47.233 | 62.400 | 19.245 | 1.00 | 9.69 | A |
| ATOM | 682 | CG | LEU | A | 111 | 46.511 | 62.066 | 17.933 | 1.00 | 10.41 | A |
| ATOM | 683 | CD1 | LEU | A | 111 | 47.335 | 62.524 | 16.739 | 1.00 | 10.20 | A |
| ATOM | 684 | CD2 | LEU | A | 111 | 46.261 | 60.566 | 17.865 | 1.00 | 11.30 | A |
| ATOM | 685 | C | LEU | A | 111 | 47.253 | 62.049 | 21.741 | 1.00 | 10.20 | A |
| ATOM | 686 | O | LEU | A | 111 | 47.695 | 60.971 | 22.138 | 1.00 | 10.28 | A |
| ATOM | 687 | N | SER | A | 112 | 47.490 | 63.196 | 22.371 | 1.00 | 10.82 | A |
| ATOM | 688 | CA | SER | A | 112 | 48.305 | 63.227 | 23.579 | 1.00 | 12.22 | A |
| ATOM | 689 | CB | SER | A | 112 | 48.593 | 64.668 | 23.993 | 1.00 | 13.20 | A |
| ATOM | 690 | OG | SER | A | 112 | 49.388 | 64.701 | 25.165 | 1.00 | 16.62 | A |
| ATOM | 691 | C | SER | A | 112 | 47.586 | 62.502 | 24.710 | 1.00 | 11.85 | A |
| ATOM | 692 | O | SER | A | 112 | 48.193 | 61.735 | 25.464 | 1.00 | 11.71 | A |
| ATOM | 693 | N | ASN | A | 113 | 46.285 | 62.737 | 24.830 | 1.00 | 11.70 | A |
| ATOM | 694 | CA | ASN | A | 113 | 45.535 | 62.080 | 25.886 | 1.00 | 12.34 | A |
| ATOM | 695 | CB | ASN | A | 113 | 44.252 | 62.858 | 26.187 | 1.00 | 12.62 | A |
| ATOM | 696 | CG | ASN | A | 113 | 44.546 | 64.218 | 26.802 | 1.00 | 14.17 | A |
| ATOM | 697 | OD1 | ASN | A | 113 | 45.603 | 64.414 | 27.404 | 1.00 | 16.67 | A |
| ATOM | 698 | ND2 | ASN | A | 113 | 43.620 | 65.155 | 26.663 | 1.00 | 15.15 | A |
| ATOM | 699 | C | ASN | A | 113 | 45.254 | 60.616 | 25.558 | 1.00 | 12.29 | A |
| ATOM | 700 | O | ASN | A | 113 | 45.082 | 59.797 | 26.460 | 1.00 | 12.15 | A |
| ATOM | 701 | N | ALA | A | 114 | 45.230 | 60.275 | 24.272 | 1.00 | 11.71 | A |
| ATOM | 702 | CA | ALA | A | 114 | 45.014 | 58.885 | 23.885 | 1.00 | 11.74 | A |
| ATOM | 703 | CB | ALA | A | 114 | 44.847 | 58.773 | 22.373 | 1.00 | 10.96 | A |
| ATOM | 704 | C | ALA | A | 114 | 46.240 | 58.097 | 24.332 | 1.00 | 11.77 | A |
| ATOM | 705 | O | ALA | A | 114 | 46.129 | 56.983 | 24.846 | 1.00 | 11.90 | A |
| ATOM | 706 | N | LEU | A | 115 | 47.415 | 58.688 | 24.139 | 1.00 | 12.06 | A |
| ATOM | 707 | CA | LEU | A | 115 | 48.663 | 58.045 | 24.517 | 1.00 | 12.83 | A |
| ATOM | 708 | CB | LEU | A | 115 | 49.854 | 58.922 | 24.114 | 1.00 | 13.32 | A |
| ATOM | 709 | CG | LEU | A | 115 | 51.247 | 58.411 | 24.497 | 1.00 | 13.43 | A |
| ATOM | 710 | CD1 | LEU | A | 115 | 51.472 | 57.025 | 23.924 | 1.00 | 13.50 | A |
| ATOM | 711 | CD2 | LEU | A | 115 | 52.301 | 59.368 | 23.984 | 1.00 | 14.02 | A |
| ATOM | 712 | C | LEU | A | 115 | 48.696 | 57.788 | 26.019 | 1.00 | 13.61 | A |
| ATOM | 713 | O | LEU | A | 115 | 49.035 | 56.692 | 26.467 | 1.00 | 13.52 | A |
| ATOM | 714 | N | ARG | A | 116 | 48.328 | 58.801 | 26.792 | 1.00 | 14.73 | A |
| ATOM | 715 | CA | ARG | A | 116 | 48.323 | 58.683 | 28.243 | 1.00 | 16.31 | A |
| ATOM | 716 | CB | ARG | A | 116 | 48.074 | 60.057 | 28.870 | 1.00 | 20.55 | A |
| ATOM | 717 | CG | ARG | A | 116 | 49.189 | 61.051 | 28.594 | 1.00 | 27.46 | A |
| ATOM | 718 | CD | ARG | A | 116 | 48.820 | 62.464 | 29.011 | 1.00 | 32.63 | A |
| ATOM | 719 | NE | ARG | A | 116 | 49.890 | 63.410 | 28.707 | 1.00 | 36.87 | A |
| ATOM | 720 | CZ | ARG | A | 116 | 49.786 | 64.728 | 28.840 | 1.00 | 39.09 | A |
| ATOM | 721 | NH1 | ARG | A | 116 | 48.654 | 65.265 | 29.274 | 1.00 | 40.46 | A |
| ATOM | 722 | NH2 | ARG | A | 116 | 50.815 | 65.510 | 28.542 | 1.00 | 40.54 | A |
| ATOM | 723 | C | ARG | A | 116 | 47.289 | 57.684 | 28.753 | 1.00 | 15.28 | A |
| ATOM | 724 | O | ARG | A | 116 | 47.615 | 56.783 | 29.529 | 1.00 | 14.55 | A |
| ATOM | 725 | N | HIS | A | 117 | 46.045 | 57.833 | 28.311 | 1.00 | 14.65 | A |
| ATOM | 726 | CA | HIS | A | 117 | 44.978 | 56.946 | 28.758 | 1.00 | 14.93 | A |
| ATOM | 727 | CB | HIS | A | 117 | 43.626 | 57.505 | 28.326 | 1.00 | 15.81 | A |
| ATOM | 728 | CG | HIS | A | 117 | 43.174 | 58.659 | 29.164 | 1.00 | 18.36 | A |
| ATOM | 729 | CD2 | HIS | A | 117 | 43.285 | 59.995 | 28.977 | 1.00 | 19.28 | A |
| ATOM | 730 | ND1 | HIS | A | 117 | 42.608 | 58.492 | 30.411 | 1.00 | 18.96 | A |
| ATOM | 731 | CE1 | HIS | A | 117 | 42.394 | 59.677 | 30.957 | 1.00 | 19.96 | A |
| ATOM | 732 | NE2 | HIS | A | 117 | 42.797 | 60.605 | 30.108 | 1.00 | 19.61 | A |
| ATOM | 733 | C | HIS | A | 117 | 45.120 | 55.494 | 28.337 | 1.00 | 14.42 | A |
| ATOM | 734 | O | HIS | A | 117 | 44.809 | 54.598 | 29.113 | 1.00 | 13.52 | A |
| ATOM | 735 | N | LEU | A | 118 | 45.585 | 55.248 | 27.119 | 1.00 | 13.01 | A |
| ATOM | 736 | CA | LEU | A | 118 | 45.769 | 53.876 | 26.669 | 1.00 | 13.05 | A |
| ATOM | 737 | CB | LEU | A | 118 | 46.041 | 53.838 | 25.161 | 1.00 | 12.57 | A |
| ATOM | 738 | CG | LEU | A | 118 | 44.841 | 54.238 | 24.292 | 1.00 | 14.54 | A |
| ATOM | 739 | CD1 | LEU | A | 118 | 45.260 | 54.333 | 22.832 | 1.00 | 14.69 | A |
| ATOM | 740 | CD2 | LEU | A | 118 | 43.728 | 53.215 | 24.456 | 1.00 | 14.25 | A |
| ATOM | 741 | C | LEU | A | 118 | 46.930 | 53.265 | 27.445 | 1.00 | 12.95 | A |
| ATOM | 742 | O | LEU | A | 118 | 46.867 | 52.112 | 27.877 | 1.00 | 13.64 | A |
| ATOM | 743 | N | HIS | A | 119 | 47.988 | 54.044 | 27.639 | 1.00 | 13.37 | A |
| ATOM | 744 | CA | HIS | A | 119 | 49.140 | 53.560 | 28.383 | 1.00 | 13.90 | A |
| ATOM | 745 | CB | HIS | A | 119 | 50.209 | 54.657 | 28.465 | 1.00 | 15.67 | A |
| ATOM | 746 | CG | HIS | A | 119 | 51.375 | 54.311 | 29.338 | 1.00 | 18.23 | A |
| ATOM | 747 | CD2 | HIS | A | 119 | 52.589 | 53.792 | 29.039 | 1.00 | 19.68 | A |
| ATOM | 748 | ND1 | HIS | A | 119 | 51.363 | 54.493 | 30.705 | 1.00 | 20.08 | A |
| ATOM | 749 | CE1 | HIS | A | 119 | 52.521 | 54.104 | 31.209 | 1.00 | 20.46 | A |
| ATOM | 750 | NE2 | HIS | A | 119 | 53.283 | 53.673 | 30.219 | 1.00 | 20.35 | A |
| ATOM | 751 | C | HIS | A | 119 | 48.716 | 53.131 | 29.788 | 1.00 | 14.72 | A |
| ATOM | 752 | O | HIS | A | 119 | 49.100 | 52.061 | 30.255 | 1.00 | 15.00 | A |
| ATOM | 753 | N | ASP | A | 120 | 47.901 | 53.954 | 30.444 | 1.00 | 14.48 | A |
| ATOM | 754 | CA | ASP | A | 120 | 47.453 | 53.664 | 31.808 | 1.00 | 14.75 | A |
| ATOM | 755 | CB | ASP | A | 120 | 47.077 | 54.964 | 32.523 | 1.00 | 15.77 | A |
| ATOM | 756 | CG | ASP | A | 120 | 48.267 | 55.877 | 32.737 | 1.00 | 16.92 | A |
| ATOM | 757 | OD1 | ASP | A | 120 | 49.409 | 55.375 | 32.760 | 1.00 | 19.17 | A |
| ATOM | 758 | OD2 | ASP | A | 120 | 48.060 | 57.097 | 32.902 | 1.00 | 19.89 | A |
| ATOM | 759 | C | ASP | A | 120 | 46.305 | 52.666 | 31.976 | 1.00 | 14.84 | A |
| ATOM | 760 | O | ASP | A | 120 | 46.051 | 52.201 | 33.090 | 1.00 | 14.82 | A |
| ATOM | 761 | N | ASN | A | 121 | 45.613 | 52.340 | 30.888 | 1.00 | 13.52 | A |
| ATOM | 762 | CA | AEN | A | 121 | 44.492 | 51.402 | 30.937 | 1.00 | 13.53 | A |
| ATOM | 763 | CB | ASN | A | 121 | 43.171 | 52.152 | 30.762 | 1.00 | 13.80 | A |
| ATOM | 764 | CG | ASN | A | 121 | 42.971 | 53.227 | 31.815 | 1.00 | 13.90 | A |
| ATOM | 765 | OD1 | ASN | A | 121 | 43.327 | 54.394 | 31.615 | 1.00 | 15.49 | A |
| ATOM | 766 | ND2 | ASN | A | 121 | 42.416 | 52.833 | 32.957 | 1.00 | 13.19 | A |
| ATOM | 767 | C | ASN | A | 121 | 44.673 | 50.374 | 29.827 | 1.00 | 14.09 | A |
| ATOM | 768 | O | ASN | A | 121 | 44.160 | 50.534 | 28.721 | 1.00 | 13.05 | A |
| ATOM | 769 | N | PRO | A | 122 | 45.406 | 49.290 | 30.121 | 1.00 | 14.55 | A |
| ATOM | 770 | CD | PRO | A | 122 | 45.858 | 48.944 | 31.481 | 1.00 | 15.39 | A |
| ATOM | 771 | CA | PRO | A | 122 | 45.704 | 48.200 | 29.187 | 1.00 | 14.85 | A |
| ATOM | 772 | CB | PRO | A | 122 | 46.410 | 47.169 | 30.072 | 1.00 | 15.58 | A |
| ATOM | 773 | CG | PRO | A | 122 | 45.853 | 47.446 | 31.436 | 1.00 | 17.22 | A |
| ATOM | 774 | C | PRO | A | 122 | 44.565 | 47.592 | 28.371 | 1.00 | 15.15 | A |
| ATOM | 775 | O | PRO | A | 122 | 44.795 | 47.126 | 27.254 | 1.00 | 15.89 | A |
| ATOM | 776 | N | GLU | A | 123 | 43.348 | 47.588 | 28.908 | 1.00 | 15.16 | A |
| ATOM | 777 | CA | GLU | A | 123 | 42.218 | 47.015 | 28.179 | 1.00 | 15.87 | A |
| ATOM | 778 | CB | GLU | A | 123 | 41.214 | 46.386 | 29.150 | 1.00 | 18.34 | A |
| ATOM | 779 | CG | GLU | A | 123 | 41.622 | 45.016 | 29.679 | 1.00 | 23.44 | A |
| ATOM | 780 | CD | GLU | A | 123 | 42.880 | 45.055 | 30.520 | 1.00 | 26.21 | A |
| ATOM | 781 | OE1 | GLU | A | 123 | 42.873 | 45.729 | 31.571 | 1.00 | 28.94 | A |
| ATOM | 782 | OE2 | GLU | A | 123 | 43.877 | 44.409 | 30.131 | 1.00 | 29.25 | A |
| ATOM | 783 | C | GLU | A | 123 | 41.490 | 48.004 | 27.269 | 1.00 | 14.24 | A |
| ATOM | 784 | O | GLU | A | 123 | 40.654 | 47.603 | 26.460 | 1.00 | 14.23 | A |
| ATOM | 785 | N | MSE | A | 124 | 41.798 | 49.290 | 27.401 | 1.00 | 12.95 | A |
| ATOM | 786 | CA | MSE | A | 124 | 41.158 | 50.303 | 26.566 | 1.00 | 12.17 | A |
| ATOM | 787 | CB | MSE | A | 124 | 41.390 | 51.699 | 27.153 | 1.00 | 13.28 | A |
| ATOM | 788 | CG | MSE | A | 124 | 40.655 | 52.810 | 26.411 | 1.00 | 14.49 | A |
| ATOM | 789 | SE | MSE | A | 124 | 38.739 | 52.530 | 26.354 | 1.00 | 19.78 | A |
| ATOM | 790 | CE | MSE | A | 124 | 38.233 | 54.129 | 25.400 | 1.00 | 15.56 | A |
| ATOM | 791 | C | MSE | A | 124 | 41.740 | 50.220 | 25.155 | 1.00 | 12.04 | A |
| ATOM | 792 | O | MSE | A | 124 | 42.918 | 49.903 | 24.983 | 1.00 | 11.44 | A |
| ATOM | 793 | N | LYS | A | 125 | 40.904 | 50.504 | 24.157 | 1.00 | 10.65 | A |
| ATOM | 794 | CA | LYS | A | 125 | 41.310 | 50.451 | 22.751 | 1.00 | 11.22 | A |
| ATOM | 795 | CB | LYS | A | 125 | 40.634 | 49.267 | 22.056 | 1.00 | 12.76 | A |
| ATOM | 796 | CG | LYS | A | 125 | 40.903 | 47.921 | 22.714 | 1.00 | 15.07 | A |
| ATOM | 797 | CD | LYS | A | 125 | 42.347 | 47.489 | 22.541 | 1.00 | 17.58 | A |
| ATOM | 798 | CE | LYS | A | 125 | 42.641 | 46.222 | 23.336 | 1.00 | 19.16 | A |
| ATOM | 799 | NZ | LYS | A | 125 | 41.712 | 45.118 | 22.986 | 1.00 | 20.18 | A |
| ATOM | 800 | C | LYS | A | 125 | 40.933 | 51.742 | 22.029 | 1.00 | 11.10 | A |
| ATOM | 801 | O | LYS | A | 125 | 40.101 | 52.512 | 22.513 | 1.00 | 10.44 | A |
| ATOM | 802 | N | PHE | A | 126 | 41.527 | 51.963 | 20.858 | 1.00 | 9.90 | A |
| ATOM | 803 | CA | PHE | A | 126 | 41.268 | 53.180 | 20.091 | 1.00 | 9.14 | A |
| ATOM | 804 | CB | PHE | A | 126 | 42.037 | 54.340 | 20.751 | 1.00 | 9.51 | A |
| ATOM | 805 | CG | PHE | A | 126 | 41.681 | 55.714 | 20.236 | 1.00 | 8.79 | A |
| ATOM | 806 | CD1 | PHE | A | 126 | 40.364 | 56.163 | 20.235 | 1.00 | 9.46 | A |
| ATOM | 807 | CD2 | PHE | A | 126 | 42.689 | 56.594 | 19.837 | 1.00 | 9.29 | A |
| ATOM | 808 | CE1 | PHE | A | 126 | 40.053 | 57.473 | 19.852 | 1.00 | 9.45 | A |
| ATOM | 809 | CE2 | PHE | A | 126 | 42.392 | 57.901 | 19.452 | 1.00 | 9.40 | A |
| ATOM | 810 | CZ | PHE | A | 126 | 41.069 | 58.343 | 19.461 | 1.00 | 8.66 | A |
| ATOM | 811 | C | PHE | A | 126 | 41.759 | 52.968 | 18.657 | 1.00 | 9.49 | A |
| ATOM | 812 | O | PHE | A | 126 | 42.823 | 52.385 | 18.448 | 1.00 | 9.51 | A |
| ATOM | 813 | N | ILE | A | 127 | 40.982 | 53.413 | 17.673 | 1.00 | 8.68 | A |
| ATOM | 814 | CA | ILE | A | 127 | 41.420 | 53.291 | 16.282 | 1.00 | 8.06 | A |
| ATOM | 815 | CB | ILE | A | 127 | 40.391 | 52.546 | 15.381 | 1.00 | 8.73 | A |
| ATOM | 816 | CG2 | ILE | A | 127 | 40.280 | 51.089 | 15.821 | 1.00 | 9.17 | A |
| ATOM | 817 | CG1 | ILE | A | 127 | 39.027 | 53.238 | 15.415 | 1.00 | 8.60 | A |
| ATOM | 818 | CD1 | ILE | A | 127 | 38.045 | 52.674 | 14.387 | 1.00 | 9.58 | A |
| ATOM | 819 | C | ILE | A | 127 | 41.690 | 54.679 | 15.706 | 1.00 | 8.78 | A |
| ATOM | 820 | O | ILE | A | 127 | 41.069 | 55.664 | 16.120 | 1.00 | 8.81 | A |
| ATOM | 821 | N | TRP | A | 128 | 42.631 | 54.757 | 14.770 | 1.00 | 8.59 | A |
| ATOM | 822 | CA | TRP | A | 128 | 42.997 | 56.029 | 14.156 | 1.00 | 8.27 | A |
| ATOM | 823 | CB | TRP | A | 128 | 44.323 | 56.530 | 14.724 | 1.00 | 8.26 | A |
| ATOM | 824 | CG | TRP | A | 128 | 44.564 | 57.952 | 14.381 | 1.00 | 8.27 | A |
| ATOM | 825 | CD2 | TRP | A | 128 | 44.001 | 59.084 | 15.044 | 1.00 | 8.03 | A |
| ATOM | 826 | CE2 | TRP | A | 128 | 44.411 | 60.233 | 14.332 | 1.00 | 8.27 | A |
| ATOM | 827 | CE3 | TRP | A | 128 | 43.181 | 59.241 | 16.172 | 1.00 | 8.19 | A |
| ATOM | 828 | CD1 | TRP | A | 128 | 45.282 | 58.440 | 13.324 | 1.00 | 8.35 | A |
| ATOM | 829 | NE1 | TRP | A | 128 | 45.192 | 59.812 | 13.287 | 1.00 | 8.32 | A |
| ATOM | 830 | CZ2 | TRP | A | 128 | 44.031 | 61.525 | 14.711 | 1.00 | 9.09 | A |
| ATOM | 831 | CZ3 | TRP | A | 128 | 42.802 | 60.525 | 16.549 | 1.00 | 8.49 | A |
| ATOM | 832 | CH2 | TRP | A | 128 | 43.229 | 61.651 | 15.817 | 1.00 | 9.77 | A |
| ATOM | 833 | C | TRP | A | 128 | 43.115 | 55.889 | 12.644 | 1.00 | 8.27 | A |
| ATOM | 834 | O | TRP | A | 128 | 43.754 | 54.958 | 12.153 | 1.00 | 8.12 | A |
| ATOM | 835 | N | ALA | A | 129 | 42.534 | 56.839 | 11.912 | 1.00 | 8.70 | A |
| ATOM | 836 | CA | ALA | A | 129 | 42.549 | 56.784 | 10.449 | 1.00 | 9.71 | A |
| ATOM | 837 | CB | ALA | A | 129 | 41.125 | 56.886 | 9.932 | 1.00 | 10.43 | A |
| ATOM | 838 | C | ALA | A | 129 | 43.414 | 57.791 | 9.694 | 1.00 | 10.28 | A |
| ATOM | 839 | O | ALA | A | 129 | 44.088 | 57.422 | 8.734 | 1.00 | 11.35 | A |
| ATOM | 840 | N | GLU | A | 130 | 43.387 | 59.054 | 10.114 | 1.00 | 10.58 | A |
| ATOM | 841 | CA | GLU | A | 130 | 44.116 | 60.124 | 9.426 | 1.00 | 10.04 | A |
| ATOM | 842 | CB | GLU | A | 130 | 43.411 | 61.461 | 9.678 | 1.00 | 11.26 | A |
| ATOM | 843 | CG | GLU | A | 130 | 41.913 | 61.457 | 9.368 | 1.00 | 11.65 | A |
| ATOM | 844 | CD | GLU | A | 130 | 41.064 | 60.935 | 10.515 | 1.00 | 13.66 | A |
| ATOM | 845 | OE1 | GLU | A | 130 | 41.629 | 60.582 | 11.573 | 1.00 | 14.42 | A |
| ATOM | 846 | OE2 | GLU | A | 130 | 39.823 | 60.885 | 10.361 | 1.00 | 15.28 | A |
| ATOM | 847 | C | GLU | A | 130 | 45.596 | 60.259 | 9.770 | 1.00 | 10.03 | A |
| ATOM | 848 | O | GLU | A | 130 | 45.962 | 60.900 | 10.755 | 1.00 | 9.29 | A |
| ATOM | 849 | N | ILE | A | 131 | 46.454 | 59.692 | 8.927 | 1.00 | 9.13 | A |
| ATOM | 850 | CA | ILE | A | 131 | 47.890 | 59.737 | 9.176 | 1.00 | 9.11 | A |
| ATOM | 851 | CB | ILE | A | 131 | 48.618 | 58.700 | 8.292 | 1.00 | 9.01 | A |
| ATOM | 852 | CG2 | ILE | A | 131 | 50.109 | 58.686 | 8.606 | 1.00 | 9.14 | A |
| ATOM | 853 | CG1 | ILE | A | 131 | 48.019 | 57.311 | 8.555 | 1.00 | 8.24 | A |
| ATOM | 854 | CD1 | ILE | A | 131 | 47.979 | 56.917 | 10.038 | 1.00 | 9.81 | A |
| ATOM | 855 | C | ILE | A | 131 | 48.518 | 61.127 | 9.030 | 1.00 | 9.70 | A |
| ATOM | 856 | O | ILE | A | 131 | 49.559 | 61.401 | 9.632 | 1.00 | 9.54 | A |
| ATOM | 857 | N | SER | A | 132 | 47.900 | 62.012 | 8.251 | 1.00 | 7.89 | A |
| ATOM | 858 | CA | SER | A | 132 | 48.432 | 63.369 | 8.125 | 1.00 | 8.13 | A |
| ATOM | 859 | CB | SER | A | 132 | 47.508 | 64.231 | 7.254 | 1.00 | 8.35 | A |
| ATOM | 860 | OG | SER | A | 132 | 46.173 | 64.210 | 7.732 | 1.00 | 9.16 | A |
| ATOM | 861 | C | SER | A | 132 | 48.546 | 63.970 | 9.532 | 1.00 | 8.70 | A |
| ATOM | 862 | O | SER | A | 132 | 49.561 | 64.576 | 9.878 | 1.00 | 8.62 | A |
| ATOM | 863 | N | TYR | A | 133 | 47.507 | 63.785 | 10.342 | 1.00 | 8.09 | A |
| ATOM | 864 | CA | TYR | A | 133 | 47.495 | 64.289 | 11.715 | 1.00 | 8.79 | A |
| ATOM | 865 | CB | TYR | A | 133 | 46.093 | 64.185 | 12.317 | 1.00 | 8.24 | A |
| ATOM | 866 | CG | TYR | A | 133 | 45.175 | 65.342 | 12.002 | 1.00 | 7.35 | A |
| ATOM | 867 | CD1 | TYR | A | 133 | 43.908 | 65.117 | 11.476 | 1.00 | 8.15 | A |
| ATOM | 868 | CE1 | TYR | A | 133 | 43.035 | 66.165 | 11.223 | 1.00 | 9.16 | A |
| ATOM | 869 | CD2 | TYR | A | 133 | 45.556 | 66.659 | 12.271 | 1.00 | 8.71 | A |
| ATOM | 870 | CE2 | TYR | A | 133 | 44.688 | 67.722 | 12.020 | 1.00 | 9.71 | A |
| ATOM | 871 | CZ | TYR | A | 133 | 43.430 | 67.466 | 11.497 | 1.00 | 9.62 | A |
| ATOM | 872 | OH | TYR | A | 133 | 42.562 | 68.503 | 11.238 | 1.00 | 10.12 | A |
| ATOM | 873 | C | TYR | A | 133 | 48.449 | 63.512 | 12.616 | 1.00 | 9.14 | A |
| ATOM | 874 | O | TYR | A | 133 | 49.164 | 64.103 | 13.430 | 1.00 | 9.28 | A |
| ATOM | 875 | N | PHE | A | 134 | 48.452 | 62.189 | 12.485 | 1.00 | 8.42 | A |
| ATOM | 876 | CA | PHE | A | 134 | 49.312 | 61.379 | 13.335 | 1.00 | 9.57 | A |
| ATOM | 877 | CB | PHE | A | 134 | 49.070 | 59.889 | 13.112 | 1.00 | 8.94 | A |
| ATOM | 878 | CG | PHE | A | 134 | 49.609 | 59.032 | 14.222 | 1.00 | 9.95 | A |
| ATOM | 879 | CD1 | PHE | A | 134 | 48.867 | 58.831 | 15.384 | 1.00 | 9.54 | A |
| ATOM | 880 | CD2 | PHE | A | 134 | 50.885 | 58.487 | 14.139 | 1.00 | 10.64 | A |
| ATOM | 881 | CE1 | PHE | A | 134 | 49.393 | 58.101 | 16.448 | 1.00 | 10.14 | A |
| ATOM | 882 | CE2 | PHE | A | 134 | 51.420 | 57.756 | 15.198 | 1.00 | 10.36 | A |
| ATOM | 883 | CZ | PHE | A | 134 | 50.672 | 57.565 | 16.354 | 1.00 | 9.82 | A |
| ATOM | 884 | C | PHE | A | 134 | 50.786 | 61.689 | 13.115 | 1.00 | 10.39 | A |
| ATOM | 885 | O | PHE | A | 134 | 51.559 | 61.757 | 14.070 | 1.00 | 10.24 | A |
| ATOM | 886 | N | ALA | A | 135 | 51.177 | 61.876 | 11.859 | 1.00 | 10.93 | A |
| ATOM | 887 | CA | ALA | A | 135 | 52.567 | 62.186 | 11.551 | 1.00 | 11.81 | A |
| ATOM | 888 | CB | ALA | A | 135 | 52.786 | 62.166 | 10.042 | 1.00 | 11.65 | A |
| ATOM | 889 | C | ALA | A | 135 | 52.921 | 63.560 | 12.129 | 1.00 | 12.73 | A |
| ATOM | 890 | O | ALA | A | 135 | 54.002 | 63.745 | 12.689 | 1.00 | 14.32 | A |
| ATOM | 891 | N | ARG | A | 136 | 52.006 | 64.514 | 11.991 | 1.00 | 12.97 | A |
| ATOM | 892 | CA | ARG | A | 136 | 52.213 | 65.868 | 12.510 | 1.00 | 13.88 | A |
| ATOM | 893 | CB | ARG | A | 136 | 50.980 | 66.735 | 12.225 | 1.00 | 14.42 | A |
| ATOM | 894 | CG | ARG | A | 136 | 50.999 | 68.119 | 12.879 | 1.00 | 15.79 | A |
| ATOM | 895 | CD | ARG | A | 136 | 51.752 | 69.145 | 12.040 | 1.00 | 17.40 | A |
| ATOM | 896 | NE | ARG | A | 136 | 51.754 | 70.460 | 12.680 | 1.00 | 17.54 | A |
| ATOM | 897 | CZ | ARG | A | 136 | 52.462 | 70.754 | 13.765 | 1.00 | 17.89 | A |
| ATOM | 898 | NH1 | ARG | A | 136 | 53.230 | 69.829 | 14.325 | 1.00 | 18.15 | A |
| ATOM | 899 | NH2 | ARG | A | 136 | 52.387 | 71.966 | 14.300 | 1.00 | 16.63 | A |
| ATOM | 900 | C | ARG | A | 136 | 52.459 | 65.813 | 14.018 | 1.00 | 14.79 | A |
| ATOM | 901 | O | ARG | A | 136 | 53.299 | 66.539 | 14.552 | 1.00 | 16.05 | A |
| ATOM | 902 | N | PHE | A | 137 | 51.720 | 64.939 | 14.692 | 1.00 | 13.05 | A |
| ATOM | 903 | CA | PHE | A | 137 | 51.823 | 64.766 | 16.136 | 1.00 | 13.29 | A |
| ATOM | 904 | CB | PHE | A | 137 | 50.607 | 63.989 | 16.639 | 1.00 | 13.13 | A |
| ATOM | 905 | CG | PHE | A | 137 | 50.611 | 63.746 | 18.116 | 1.00 | 13.98 | A |
| ATOM | 906 | CD1 | PHE | A | 137 | 50.372 | 64.791 | 19.000 | 1.00 | 13.65 | A |
| ATOM | 907 | CD2 | PHE | A | 137 | 50.864 | 62.478 | 18.624 | 1.00 | 14.23 | A |
| ATOM | 908 | CE1 | PHE | A | 137 | 50.381 | 64.582 | 20.372 | 1.00 | 14.75 | A |
| ATOM | 909 | CE2 | PHE | A | 137 | 50.876 | 62.256 | 20.005 | 1.00 | 15.02 | A |
| ATOM | 910 | CZ | PHE | A | 137 | 50.633 | 63.314 | 20.875 | 1.00 | 13.04 | A |
| ATOM | 911 | C | PHE | A | 137 | 53.089 | 64.027 | 16.557 | 1.00 | 13.44 | A |
| ATOM | 912 | O | PHE | A | 137 | 53.866 | 64.509 | 17.387 | 1.00 | 14.03 | A |
| ATOM | 913 | N | TYR | A | 138 | 53.287 | 62.851 | 15.974 | 1.00 | 13.77 | A |
| ATOM | 914 | CA | TYR | A | 138 | 54.427 | 62.001 | 16.291 | 1.00 | 14.96 | A |
| ATOM | 915 | CB | TYR | A | 138 | 54.433 | 60.778 | 15.375 | 1.00 | 14.56 | A |
| ATOM | 916 | CG | TYR | A | 138 | 55.421 | 59.715 | 15.795 | 1.00 | 14.71 | A |
| ATOM | 917 | CD1 | TYR | A | 138 | 55.125 | 58.837 | 16.836 | 1.00 | 14.96 | A |
| ATOM | 918 | CE1 | TYR | A | 138 | 56.030 | 57.855 | 17.230 | 1.00 | 15.31 | A |
| ATOM | 919 | CD2 | TYR | A | 138 | 56.653 | 59.589 | 15.156 | 1.00 | 15.19 | A |
| ATOM | 920 | CE2 | TYR | A | 138 | 57.567 | 58.608 | 15.544 | 1.00 | 15.16 | A |
| ATOM | 921 | CZ | TYR | A | 138 | 57.247 | 57.746 | 16.579 | 1.00 | 15.51 | A |
| ATOM | 922 | OH | TYR | A | 138 | 58.138 | 56.769 | 16.958 | 1.00 | 17.55 | A |
| ATOM | 923 | C | TYR | A | 138 | 55.783 | 62.691 | 16.198 | 1.00 | 16.54 | A |
| ATOM | 924 | O | TYR | A | 138 | 56.625 | 62.536 | 17.084 | 1.00 | 16.24 | A |
| ATOM | 925 | N | HIS | A | 139 | 56.001 | 63.445 | 15.128 | 1.00 | 17.34 | A |
| ATOM | 926 | CA | HIS | A | 139 | 57.277 | 64.124 | 14.948 | 1.00 | 19.15 | A |
| ATOM | 927 | CB | HIS | A | 139 | 57.381 | 64.678 | 13.525 | 1.00 | 19.96 | A |
| ATOM | 928 | CG | HIS | A | 139 | 57.571 | 63.618 | 12.483 | 1.00 | 21.16 | A |
| ATOM | 929 | CD2 | HIS | A | 139 | 56.802 | 63.252 | 11.429 | 1.00 | 21.41 | A |
| ATOM | 930 | ND1 | HIS | A | 139 | 58.666 | 62.780 | 12.467 | 1.00 | 21.98 | A |
| ATOM | 931 | CE1 | HIS | A | 139 | 58.564 | 61.945 | 11.448 | 1.00 | 22.61 | A |
| ATOM | 932 | NE2 | HIS | A | 139 | 57.443 | 62.210 | 10.802 | 1.00 | 21.85 | A |
| ATOM | 933 | C | HIS | A | 139 | 57.546 | 65.221 | 15.975 | 1.00 | 19.75 | A |
| ATOM | 934 | O | HIS | A | 139 | 58.683 | 65.660 | 16.133 | 1.00 | 20.84 | A |
| ATOM | 935 | N | ASP | A | 140 | 56.505 | 65.660 | 16.675 | 1.00 | 18.81 | A |
| ATOM | 936 | CA | ASP | A | 140 | 56.658 | 66.692 | 17.697 | 1.00 | 18.99 | A |
| ATOM | 937 | CB | ASP | A | 140 | 55.438 | 67.614 | 17.705 | 1.00 | 20.32 | A |
| ATOM | 938 | CG | ASP | A | 140 | 55.559 | 68.742 | 16.702 | 1.00 | 21.99 | A |
| ATOM | 939 | OD1 | ASP | A | 140 | 56.308 | 68.579 | 15.717 | 1.00 | 23.53 | A |
| ATOM | 940 | OD2 | ASP | A | 140 | 54.901 | 69.786 | 16.894 | 1.00 | 22.73 | A |
| ATOM | 941 | C | ASP | A | 140 | 56.855 | 66.078 | 19.080 | 1.00 | 18.52 | A |
| ATOM | 942 | O | ASP | A | 140 | 57.154 | 66.781 | 20.048 | 1.00 | 18.42 | A |
| ATOM | 943 | N | LEU | A | 141 | 56.692 | 64.762 | 19.166 | 1.00 | 18.16 | A |
| ATOM | 944 | CA | LEU | A | 141 | 56.851 | 64.043 | 20.426 | 1.00 | 18.15 | A |
| ATOM | 945 | CB | LEU | A | 141 | 56.171 | 62.674 | 20.355 | 1.00 | 17.96 | A |
| ATOM | 946 | CG | LEU | A | 141 | 54.651 | 62.547 | 20.381 | 1.00 | 17.86 | A |
| ATOM | 947 | CD1 | LEU | A | 141 | 54.285 | 61.069 | 20.292 | 1.00 | 17.13 | A |
| ATOM | 948 | CD2 | LEU | A | 141 | 54.097 | 63.154 | 21.662 | 1.00 | 17.42 | A |
| ATOM | 949 | C | LEU | A | 141 | 58.306 | 63.816 | 20.805 | 1.00 | 19.03 | A |
| ATOM | 950 | O | LEU | A | 141 | 59.168 | 63.651 | 19.943 | 1.00 | 18.72 | A |
| ATOM | 951 | N | GLY | A | 142 | 58.569 | 63.802 | 22.108 | 1.00 | 20.19 | A |
| ATOM | 952 | CA | GLY | A | 142 | 59.913 | 63.545 | 22.585 | 1.00 | 21.21 | A |
| ATOM | 953 | C | GLY | A | 142 | 60.161 | 62.057 | 22.424 | 1.00 | 22.12 | A |
| ATOM | 954 | O | GLY | A | 142 | 59.209 | 61.283 | 22.295 | 1.00 | 21.38 | A |
| ATOM | 955 | N | GLU | A | 143 | 61.425 | 61.646 | 22.437 | 1.00 | 23.15 | A |
| ATOM | 956 | CA | GLU | A | 143 | 61.772 | 60.239 | 22.267 | 1.00 | 24.67 | A |
| ATOM | 957 | CB | GLU | A | 143 | 63.287 | 60.052 | 22.381 | 1.00 | 27.38 | A |
| ATOM | 958 | CG | GLU | A | 143 | 63.763 | 58.660 | 21.997 | 1.00 | 30.77 | A |
| ATOM | 959 | CD | GLU | A | 143 | 63.303 | 58.253 | 20.609 | 1.00 | 32.52 | A |
| ATOM | 960 | OE1 | GLU | A | 143 | 63.586 | 58.993 | 19.643 | 1.00 | 33.98 | A |
| ATOM | 961 | OE2 | GLU | A | 143 | 62.656 | 57.192 | 20.484 | 1.00 | 33.85 | A |
| ATOM | 962 | C | GLU | A | 143 | 61.066 | 59.311 | 23.252 | 1.00 | 24.07 | A |
| ATOM | 963 | O | GLU | A | 143 | 60.625 | 58.225 | 22.880 | 1.00 | 23.09 | A |
| ATOM | 964 | N | ASN | A | 144 | 60.958 | 59.736 | 24.506 | 1.00 | 23.61 | A |
| ATOM | 965 | CA | ASN | A | 144 | 60.302 | 58.924 | 25.525 | 1.00 | 23.85 | A |
| ATOM | 966 | CB | ASN | A | 144 | 60.329 | 59.652 | 26.874 | 1.00 | 25.76 | A |
| ATOM | 967 | CG | ASN | A | 144 | 59.589 | 58.897 | 27.967 | 1.00 | 27.79 | A |
| ATOM | 968 | OD1 | ASN | A | 144 | 58.360 | 58.797 | 27.949 | 1.00 | 28.98 | A |
| ATOM | 969 | ND2 | ASN | A | 144 | 60.338 | 58.359 | 28.923 | 1.00 | 28.61 | A |
| ATOM | 970 | C | ASN | A | 144 | 58.864 | 58.610 | 25.124 | 1.00 | 22.68 | A |
| ATOM | 971 | O | ASN | A | 144 | 58.425 | 57.462 | 25.202 | 1.00 | 22.15 | A |
| ATOM | 972 | N | LYS | A | 145 | 58.139 | 59.634 | 24.682 | 1.00 | 21.73 | A |
| ATOM | 973 | CA | LYS | A | 145 | 56.749 | 59.470 | 24.270 | 1.00 | 20.84 | A |
| ATOM | 974 | CB | LYS | A | 145 | 56.073 | 60.837 | 24.132 | 1.00 | 22.57 | A |
| ATOM | 975 | CG | LYS | A | 145 | 55.854 | 61.561 | 25.455 | 1.00 | 24.93 | A |
| ATOM | 976 | CD | LYS | A | 145 | 54.914 | 60.778 | 26.361 | 1.00 | 27.02 | A |
| ATOM | 977 | CE | LYS | A | 145 | 54.640 | 61.523 | 27.661 | 1.00 | 28.49 | A |
| ATOM | 978 | NZ | LYS | A | 145 | 55.882 | 61.756 | 28.449 | 1.00 | 29.61 | A |
| ATOM | 979 | C | LYS | A | 145 | 56.617 | 58.689 | 22.965 | 1.00 | 19.52 | A |
| ATOM | 980 | O | LYS | A | 145 | 55.654 | 57.940 | 22.784 | 1.00 | 18.64 | A |
| ATOM | 981 | N | LYS | A | 146 | 57.571 | 58.866 | 22.054 | 1.00 | 18.73 | A |
| ATOM | 982 | CA | LYS | A | 146 | 57.535 | 58.137 | 20.788 | 1.00 | 17.79 | A |
| ATOM | 983 | CB | LYS | A | 146 | 58.730 | 58.502 | 19.898 | 1.00 | 17.55 | A |
| ATOM | 984 | CG | LYS | A | 146 | 58.589 | 59.813 | 19.139 | 1.00 | 17.75 | A |
| ATOM | 985 | CD | LYS | A | 146 | 59.758 | 60.012 | 18.181 | 1.00 | 18.32 | A |
| ATOM | 986 | CE | LYS | A | 146 | 59.592 | 61.275 | 17.344 | 1.00 | 18.96 | A |
| ATOM | 987 | NZ | LYS | A | 146 | 60.778 | 61.526 | 16.481 | 1.00 | 22.20 | A |
| ATOM | 988 | C | LYS | A | 146 | 57.576 | 56.647 | 21.097 | 1.00 | 17.36 | A |
| ATOM | 989 | O | LYS | A | 146 | 56.859 | 55.856 | 20.487 | 1.00 | 16.67 | A |
| ATOM | 990 | N | LEU | A | 147 | 58.419 | 56.273 | 22.055 | 1.00 | 17.41 | A |
| ATOM | 991 | CA | LEU | A | 147 | 58.557 | 54.880 | 22.461 | 1.00 | 17.08 | A |
| ATOM | 992 | CB | LEU | A | 147 | 59.735 | 54.727 | 23.429 | 1.00 | 18.23 | A |
| ATOM | 993 | CG | LEU | A | 147 | 61.111 | 54.969 | 22.802 | 1.00 | 19.30 | A |
| ATOM | 994 | CD1 | LEU | A | 147 | 62.186 | 54.935 | 23.876 | 1.00 | 19.92 | A |
| ATOM | 995 | CD2 | LEU | A | 147 | 61.380 | 53.909 | 21.741 | 1.00 | 20.31 | A |
| ATOM | 996 | C | LEU | A | 147 | 57.273 | 54.362 | 23.103 | 1.00 | 16.49 | A |
| ATOM | 997 | O | LEU | A | 147 | 56.855 | 53.235 | 22.841 | 1.00 | 15.40 | A |
| ATOM | 998 | N | GLN | A | 148 | 56.645 | 55.179 | 23.944 | 1.00 | 16.51 | A |
| ATOM | 999 | CA | GLN | A | 148 | 55.395 | 54.769 | 24.572 | 1.00 | 16.83 | A |
| ATOM | 1000 | CB | GLN | A | 148 | 54.917 | 55.815 | 25.581 | 1.00 | 18.73 | A |
| ATOM | 1001 | CG | GLN | A | 148 | 55.746 | 55.891 | 26.849 | 1.00 | 21.13 | A |
| ATOM | 1002 | CD | GLN | A | 148 | 55.121 | 56.796 | 27.894 | 1.00 | 23.21 | A |
| ATOM | 1003 | OE1 | GLN | A | 148 | 55.652 | 56.951 | 28.996 | 1.00 | 25.13 | A |
| ATOM | 1004 | NE2 | GLN | A | 148 | 53.986 | 57.397 | 27.556 | 1.00 | 24.08 | A |
| ATOM | 1005 | C | GLN | A | 148 | 54.333 | 54.585 | 23.491 | 1.00 | 16.17 | A |
| ATOM | 1006 | O | GLN | A | 148 | 53.519 | 53.666 | 23.556 | 1.00 | 16.06 | A |
| ATOM | 1007 | N | MSE | A | 149 | 54.350 | 55.462 | 22.493 | 1.00 | 15.44 | A |
| ATOM | 1008 | CA | MSE | A | 149 | 53.380 | 55.375 | 21.409 | 1.00 | 15.77 | A |
| ATOM | 1009 | CB | MSE | A | 149 | 53.494 | 56.595 | 20.492 | 1.00 | 17.35 | A |
| ATOM | 1010 | CG | MSE | A | 149 | 52.475 | 56.619 | 19.359 | 1.00 | 19.00 | A |
| ATOM | 1011 | SE | MSE | A | 149 | 50.649 | 56.669 | 19.994 | 1.00 | 24.67 | A |
| ATOM | 1012 | CE | MSE | A | 149 | 50.424 | 58.578 | 20.144 | 1.00 | 23.17 | A |
| ATOM | 1013 | C | MSE | A | 149 | 53.589 | 54.099 | 20.601 | 1.00 | 15.13 | A |
| ATOM | 1014 | O | MSE | A | 149 | 52.633 | 53.397 | 20.281 | 1.00 | 13.65 | A |
| ATOM | 1015 | N | LYS | A | 150 | 54.840 | 53.796 | 20.270 | 1.00 | 15.43 | A |
| ATOM | 1016 | CA | LYS | A | 150 | 55.128 | 52.594 | 19.498 | 1.00 | 15.61 | A |
| ATOM | 1017 | CB | LYS | A | 150 | 56.620 | 52.516 | 19.155 | 1.00 | 16.35 | A |
| ATOM | 1018 | CG | LYS | A | 150 | 57.081 | 53.610 | 18.200 | 1.00 | 19.37 | A |
| ATOM | 1019 | CD | LYS | A | 150 | 58.582 | 53.563 | 17.941 | 1.00 | 22.38 | A |
| ATOM | 1020 | CE | LYS | A | 150 | 58.987 | 52.322 | 17.169 | 1.00 | 24.63 | A |
| ATOM | 1021 | NZ | LYS | A | 150 | 60.441 | 52.335 | 16.840 | 1.00 | 26.82 | A |
| ATOM | 1022 | C | LYS | A | 150 | 54.702 | 51.349 | 20.266 | 1.00 | 14.98 | A |
| ATOM | 1023 | O | LYS | A | 150 | 54.265 | 50.365 | 19.672 | 1.00 | 15.43 | A |
| ATOM | 1024 | N | SER | A | 151 | 54.806 | 51.400 | 21.591 | 1.00 | 15.45 | A |
| ATOM | 1025 | CA | SER | A | 151 | 54.438 | 50.257 | 22.415 | 1.00 | 15.79 | A |
| ATOM | 1026 | CB | SER | A | 151 | 54.936 | 50.450 | 23.851 | 1.00 | 17.27 | A |
| ATOM | 1027 | OG | SER | A | 151 | 54.181 | 51.437 | 24.526 | 1.00 | 21.28 | A |
| ATOM | 1028 | C | SER | A | 151 | 52.936 | 49.975 | 22.422 | 1.00 | 15.04 | A |
| ATOM | 1029 | O | SER | A | 151 | 52.530 | 48.818 | 22.315 | 1.00 | 15.10 | A |
| ATOM | 1030 | N | ILE | A | 152 | 52.105 | 51.010 | 22.545 | 1.00 | 14.25 | A |
| ATOM | 1031 | CA | ILE | A | 152 | 50.666 | 50.771 | 22.554 | 1.00 | 13.46 | A |
| ATOM | 1032 | CB | ILE | A | 152 | 49.857 | 51.979 | 23.115 | 1.00 | 14.01 | A |
| ATOM | 1033 | CG2 | ILE | A | 152 | 50.228 | 52.203 | 24.577 | 1.00 | 14.32 | A |
| ATOM | 1034 | CG1 | ILE | A | 152 | 50.103 | 53.243 | 22.295 | 1.00 | 13.78 | A |
| ATOM | 1035 | CD1 | ILE | A | 152 | 49.179 | 54.395 | 22.676 | 1.00 | 13.84 | A |
| ATOM | 1036 | C | ILE | A | 152 | 50.149 | 50.383 | 21.172 | 1.00 | 13.41 | A |
| ATOM | 1037 | O | ILE | A | 152 | 49.048 | 49.856 | 21.044 | 1.00 | 13.62 | A |
| ATOM | 1038 | N | VAL | A | 153 | 50.942 | 50.642 | 20.136 | 1.00 | 12.90 | A |
| ATOM | 1039 | CA | VAL | A | 153 | 50.548 | 50.253 | 18.786 | 1.00 | 13.40 | A |
| ATOM | 1040 | CB | VAL | A | 153 | 51.242 | 51.122 | 17.709 | 1.00 | 13.08 | A |
| ATOM | 1041 | CG1 | VAL | A | 153 | 51.050 | 50.501 | 16.322 | 1.00 | 13.04 | A |
| ATOM | 1042 | CG2 | VAL | A | 153 | 50.665 | 52.530 | 17.737 | 1.00 | 13.27 | A |
| ATOM | 1043 | C | VAL | A | 153 | 50.965 | 48.798 | 18.610 | 1.00 | 14.30 | A |
| ATOM | 1044 | O | VAL | A | 153 | 50.195 | 47.970 | 18.121 | 1.00 | 14.16 | A |
| ATOM | 1045 | N | LYS | A | 154 | 52.182 | 48.487 | 19.038 | 1.00 | 14.99 | A |
| ATOM | 1046 | CA | LYS | A | 154 | 52.696 | 47.130 | 18.926 | 1.00 | 16.69 | A |
| ATOM | 1047 | CB | LYS | A | 154 | 54.157 | 47.079 | 19.383 | 1.00 | 17.97 | A |
| ATOM | 1048 | CG | LYS | A | 154 | 54.867 | 45.775 | 19.034 | 1.00 | 21.07 | A |
| ATOM | 1049 | CD | LYS | A | 154 | 56.327 | 45.789 | 19.466 | 1.00 | 23.29 | A |
| ATOM | 1050 | CE | LYS | A | 154 | 56.476 | 45.645 | 20.976 | 1.00 | 25.47 | A |
| ATOM | 1051 | NZ | LYS | A | 154 | 55.866 | 46.770 | 21.737 | 1.00 | 26.91 | A |
| ATOM | 1052 | C | LYS | A | 154 | 51.859 | 46.146 | 19.745 | 1.00 | 16.92 | A |
| ATOM | 1053 | O | LYS | A | 154 | 51.667 | 45.003 | 19.328 | 1.00 | 17.42 | A |
| ATOM | 1054 | N | ASN | A | 155 | 51.351 | 46.587 | 20.895 | 1.00 | 16.90 | A |
| ATOM | 1055 | CA | ASN | A | 155 | 50.546 | 45.712 | 21.752 | 1.00 | 17.32 | A |
| ATOM | 1056 | CB | ASN | A | 155 | 50.651 | 46.135 | 23.226 | 1.00 | 19.11 | A |
| ATOM | 1057 | CG | ASN | A | 155 | 49.813 | 47.363 | 23.557 | 1.00 | 20.16 | A |
| ATOM | 1058 | OD1 | ASN | A | 155 | 49.021 | 47.831 | 22.743 | 1.00 | 21.67 | A |
| ATOM | 1059 | ND2 | ASN | A | 155 | 49.980 | 47.883 | 24.772 | 1.00 | 20.93 | A |
| ATOM | 1060 | C | ASN | A | 155 | 49.076 | 45.635 | 21.341 | 1.00 | 17.02 | A |
| ATOM | 1061 | O | ASN | A | 155 | 48.293 | 44.909 | 21.949 | 1.00 | 18.03 | A |
| ATOM | 1062 | N | GLY | A | 156 | 48.700 | 46.404 | 20.324 | 1.00 | 15.86 | A |
| ATOM | 1063 | CA | GLY | A | 156 | 47.332 | 46.359 | 19.837 | 1.00 | 15.20 | A |
| ATOM | 1064 | C | GLY | A | 156 | 46.276 | 47.254 | 20.463 | 1.00 | 14.17 | A |
| ATOM | 1065 | O | GLY | A | 156 | 45.097 | 47.107 | 20.146 | 1.00 | 15.09 | A |
| ATOM | 1066 | N | GLN | A | 157 | 46.663 | 48.172 | 21.344 | 1.00 | 12.63 | A |
| ATOM | 1067 | CA | GLN | A | 157 | 45.672 | 49.058 | 21.953 | 1.00 | 11.77 | A |
| ATOM | 1068 | CB | GLN | A | 157 | 46.214 | 49.694 | 23.225 | 1.00 | 11.96 | A |
| ATOM | 1069 | CG | GLN | A | 157 | 46.326 | 48.750 | 24.399 | 1.00 | 12.30 | A |
| ATOM | 1070 | CD | GLN | A | 157 | 46.581 | 49.503 | 25.680 | 1.00 | 11.07 | A |
| ATOM | 1071 | OE1 | GLN | A | 157 | 45.677 | 50.125 | 26.244 | 1.00 | 14.09 | A |
| ATOM | 1072 | NE2 | GLN | A | 157 | 47.823 | 49.477 | 26.136 | 1.00 | 11.57 | A |
| ATOM | 1073 | C | GLN | A | 157 | 45.255 | 50.160 | 20.985 | 1.00 | 10.91 | A |
| ATOM | 1074 | O | GLN | A | 157 | 44.077 | 50.484 | 20.880 | 1.00 | 10.65 | A |
| ATOM | 1075 | N | LEU | A | 158 | 46.238 | 50.747 | 20.308 | 1.00 | 11.05 | A |
| ATOM | 1076 | CA | LEU | A | 158 | 45.987 | 51.788 | 19.320 | 1.00 | 10.93 | A |
| ATOM | 1077 | CB | LEU | A | 158 | 46.989 | 52.935 | 19.479 | 1.00 | 12.25 | A |
| ATOM | 1078 | CG | LEU | A | 158 | 46.895 | 54.117 | 18.507 | 1.00 | 14.64 | A |
| ATOM | 1079 | CD1 | LEU | A | 158 | 47.377 | 53.725 | 17.123 | 1.00 | 17.13 | A |
| ATOM | 1080 | CD2 | LEU | A | 158 | 45.471 | 54.612 | 18.461 | 1.00 | 15.49 | A |
| ATOM | 1081 | C | LEU | A | 158 | 46.177 | 51.111 | 17.971 | 1.00 | 11.18 | A |
| ATOM | 1082 | O | LEU | A | 158 | 47.258 | 50.600 | 17.671 | 1.00 | 11.17 | A |
| ATOM | 1083 | N | GLU | A | 159 | 45.126 | 51.102 | 17.161 | 1.00 | 9.90 | A |
| ATOM | 1084 | CA | GLU | A | 159 | 45.201 | 50.456 | 15.861 | 1.00 | 9.47 | A |
| ATOM | 1085 | CB | GLU | A | 159 | 44.247 | 49.263 | 15.829 | 1.00 | 10.02 | A |
| ATOM | 1086 | CG | GLU | A | 159 | 44.206 | 48.531 | 14.506 | 1.00 | 10.65 | A |
| ATOM | 1087 | CD | GLU | A | 159 | 43.223 | 47.383 | 14.527 | 1.00 | 12.18 | A |
| ATOM | 1088 | OE1 | GLU | A | 159 | 43.502 | 46.369 | 15.209 | 1.00 | 11.96 | A |
| ATOM | 1089 | OE2 | GLU | A | 159 | 42.168 | 47.502 | 13.873 | 1.00 | 11.42 | A |
| ATOM | 1090 | C | GLU | A | 159 | 44.864 | 51.409 | 14.732 | 1.00 | 8.85 | A |
| ATOM | 1091 | O | GLU | A | 159 | 43.871 | 52.133 | 14.786 | 1.00 | 9.51 | A |
| ATOM | 1092 | N | PHE | A | 160 | 45.707 | 51.409 | 13.709 | 1.00 | 8.61 | A |
| ATOM | 1093 | CA | PHE | A | 160 | 45.475 | 52.259 | 12.557 | 1.00 | 8.11 | A |
| ATOM | 1094 | CB | PHE | A | 160 | 46.802 | 52.613 | 11.881 | 1.00 | 8.37 | A |
| ATOM | 1095 | CG | PHE | A | 160 | 47.698 | 53.447 | 12.743 | 1.00 | 8.91 | A |
| ATOM | 1096 | CD1 | PHE | A | 160 | 48.761 | 52.872 | 13.437 | 1.00 | 9.51 | A |
| ATOM | 1097 | CD2 | PHE | A | 160 | 47.444 | 54.801 | 12.906 | 1.00 | 9.15 | A |
| ATOM | 1098 | CE1 | PHE | A | 160 | 49.555 | 53.643 | 14.284 | 1.00 | 9.60 | A |
| ATOM | 1099 | CE2 | PHE | A | 160 | 48.233 | 55.581 | 13.754 | 1.00 | 9.20 | A |
| ATOM | 1100 | CZ | PHE | A | 160 | 49.288 | 54.997 | 14.442 | 1.00 | 10.42 | A |
| ATOM | 1101 | C | PHE | A | 160 | 44.553 | 51.558 | 11.576 | 1.00 | 8.64 | A |
| ATOM | 1102 | O | PHE | A | 160 | 44.726 | 50.374 | 11.270 | 1.00 | 8.99 | A |
| ATOM | 1103 | N | VAL | A | 161 | 43.551 | 52.293 | 11.111 | 1.00 | 8.21 | A |
| ATOM | 1104 | CA | VAL | A | 161 | 42.603 | 51.767 | 10.147 | 1.00 | 8.58 | A |
| ATOM | 1105 | CB | VAL | A | 161 | 41.147 | 51.874 | 10.679 | 1.00 | 7.15 | A |
| ATOM | 1106 | CG1 | VAL | A | 161 | 40.961 | 50.891 | 11.846 | 1.00 | 8.25 | A |
| ATOM | 1107 | CG2 | VAL | A | 161 | 40.848 | 53.290 | 11.153 | 1.00 | 8.29 | A |
| ATOM | 1108 | C | VAL | A | 161 | 42.815 | 52.542 | 8.847 | 1.00 | 8.27 | A |
| ATOM | 1109 | O | VAL | A | 161 | 42.899 | 53.774 | 8.848 | 1.00 | 8.98 | A |
| ATOM | 1110 | N | THR | A | 162 | 42.926 | 51.788 | 7.755 | 1.00 | 8.30 | A |
| ATOM | 1111 | CA | THR | A | 162 | 43.203 | 52.294 | 6.403 | 1.00 | 8.76 | A |
| ATOM | 1112 | CB | THR | A | 162 | 42.296 | 53.473 | 5.984 | 1.00 | 8.95 | A |
| ATOM | 1113 | OG1 | THR | A | 162 | 40.920 | 53.089 | 6.084 | 1.00 | 10.09 | A |
| ATOM | 1114 | CG2 | THR | A | 162 | 42.580 | 53.855 | 4.532 | 1.00 | 10.53 | A |
| ATOM | 1115 | C | THR | A | 162 | 44.656 | 52.759 | 6.347 | 1.00 | 8.36 | A |
| ATOM | 1116 | O | THR | A | 162 | 45.478 | 52.184 | 5.633 | 1.00 | 8.75 | A |
| ATOM | 1117 | N | GLY | A | 163 | 44.972 | 53.800 | 7.107 | 1.00 | 8.59 | A |
| ATOM | 1118 | CA | GLY | A | 163 | 46.338 | 54.286 | 7.133 | 1.00 | 8.14 | A |
| ATOM | 1119 | C | GLY | A | 163 | 46.702 | 55.259 | 6.032 | 1.00 | 7.44 | A |
| ATOM | 1120 | O | GLY | A | 163 | 47.880 | 55.450 | 5.747 | 1.00 | 8.02 | A |
| ATOM | 1121 | N | GLY | A | 164 | 45.699 | 55.864 | 5.405 | 1.00 | 6.59 | A |
| ATOM | 1122 | CA | GLY | A | 164 | 45.975 | 56.836 | 4.364 | 1.00 | 7.84 | A |
| ATOM | 1123 | C | GLY | A | 164 | 46.278 | 58.189 | 4.979 | 1.00 | 7.81 | A |
| ATOM | 1124 | O | GLY | A | 164 | 46.015 | 58.419 | 6.160 | 1.00 | 7.64 | A |
| ATOM | 1125 | N | TRP | A | 165 | 46.845 | 59.092 | 4.188 | 1.00 | 6.53 | A |
| ATOM | 1126 | CA | TRP | A | 165 | 47.159 | 60.428 | 4.678 | 1.00 | 6.64 | A |
| ATOM | 1127 | CB | TRP | A | 165 | 47.767 | 61.255 | 3.545 | 1.00 | 6.57 | A |
| ATOM | 1128 | CG | TRP | A | 165 | 48.563 | 62.437 | 4.001 | 1.00 | 7.87 | A |
| ATOM | 1129 | CD2 | TRP | A | 165 | 49.760 | 62.414 | 4.791 | 1.00 | 9.42 | A |
| ATOM | 1130 | CE2 | TRP | A | 165 | 50.184 | 63.753 | 4.945 | 1.00 | 9.13 | A |
| ATOM | 1131 | CE3 | TRP | A | 165 | 50.517 | 61.392 | 5.383 | 1.00 | 9.09 | A |
| ATOM | 1132 | CD1 | TRP | A | 165 | 48.316 | 63.748 | 3.717 | 1.00 | 9.06 | A |
| ATOM | 1133 | NE1 | TRP | A | 165 | 49.286 | 64.546 | 4.281 | 1.00 | 9.55 | A |
| ATOM | 1134 | CZ2 | TRP | A | 165 | 51.334 | 64.100 | 5.670 | 1.00 | 10.93 | A |
| ATOM | 1135 | CZ3 | TRP | A | 165 | 51.662 | 61.738 | 6.103 | 1.00 | 11.93 | A |
| ATOM | 1136 | CH2 | TRP | A | 165 | 52.056 | 63.080 | 6.238 | 1.00 | 11.13 | A |
| ATOM | 1137 | C | TRP | A | 165 | 45.854 | 61.061 | 5.163 | 1.00 | 5.76 | A |
| ATOM | 1138 | O | TRP | A | 165 | 45.838 | 61.822 | 6.138 | 1.00 | 6.45 | A |
| ATOM | 1139 | N | VAL | A | 166 | 44.766 | 60.720 | 4.477 | 1.00 | 5.84 | A |
| ATOM | 1140 | CA | VAL | A | 166 | 43.430 | 61.215 | 4.800 | 1.00 | 6.47 | A |
| ATOM | 1141 | CB | VAL | A | 166 | 43.033 | 62.406 | 3.882 | 1.00 | 6.49 | A |
| ATOM | 1142 | CG1 | VAL | A | 166 | 44.041 | 63.541 | 4.018 | 1.00 | 7.93 | A |
| ATOM | 1143 | CG2 | VAL | A | 166 | 42.956 | 61.943 | 2.426 | 1.00 | 7.01 | A |
| ATOM | 1144 | C | VAL | A | 166 | 42.414 | 60.096 | 4.560 | 1.00 | 6.42 | A |
| ATOM | 1145 | O | VAL | A | 166 | 42.787 | 58.962 | 4.258 | 1.00 | 7.28 | A |
| ATOM | 1146 | N | MSE | A | 167 | 41.137 | 60.424 | 4.743 | 1.00 | 6.92 | A |
| ATOM | 1147 | CA | MSE | A | 167 | 40.022 | 59.517 | 4.458 | 1.00 | 7.79 | A |
| ATOM | 1148 | CB | MSE | A | 167 | 39.001 | 59.533 | 5.590 | 1.00 | 10.16 | A |
| ATOM | 1149 | CG | MSE | A | 167 | 37.784 | 58.669 | 5.322 | 1.00 | 10.68 | A |
| ATOM | 1150 | SE | MSE | A | 167 | 36.562 | 58.804 | 6.790 | 1.00 | 17.10 | A |
| ATOM | 1151 | CE | MSE | A | 167 | 37.703 | 58.023 | 8.131 | 1.00 | 10.95 | A |
| ATOM | 1152 | C | MSE | A | 167 | 39.494 | 60.275 | 3.245 | 1.00 | 7.78 | A |
| ATOM | 1153 | O | MSE | A | 167 | 38.701 | 61.204 | 3.368 | 1.00 | 7.60 | A |
| ATOM | 1154 | N | PRO | A | 168 | 39.919 | 59.870 | 2.045 | 1.00 | 6.75 | A |
| ATOM | 1155 | CD | PRO | A | 168 | 40.680 | 58.649 | 1.716 | 1.00 | 6.63 | A |
| ATOM | 1156 | CA | PRO | A | 168 | 39.499 | 60.558 | 0.829 | 1.00 | 7.01 | A |
| ATOM | 1157 | CB | PRO | A | 168 | 40.437 | 59.975 | −0.219 | 1.00 | 7.18 | A |
| ATOM | 1158 | CG | PRO | A | 168 | 40.514 | 58.546 | 0.200 | 1.00 | 7.01 | A |
| ATOM | 1159 | C | PRO | A | 168 | 38.070 | 60.528 | 0.355 | 1.00 | 6.98 | A |
| ATOM | 1160 | O | PRO | A | 168 | 37.304 | 59.615 | 0.657 | 1.00 | 7.17 | A |
| ATOM | 1161 | N | ASP | A | 169 | 37.732 | 61.572 | −0.395 | 1.00 | 6.85 | A |
| ATOM | 1162 | CA | ASP | A | 169 | 36.439 | 61.673 | −1.043 | 1.00 | 7.10 | A |
| ATOM | 1163 | CB | ASP | A | 169 | 36.341 | 63.006 | −1.790 | 1.00 | 7.46 | A |
| ATOM | 1164 | CG | ASP | A | 169 | 35.205 | 63.039 | −2.791 | 1.00 | 7.67 | A |
| ATOM | 1165 | OD1 | ASP | A | 169 | 34.118 | 62.511 | −2.486 | 1.00 | 8.52 | A |
| ATOM | 1166 | OD2 | ASP | A | 169 | 35.399 | 63.609 | −3.885 | 1.00 | 8.86 | A |
| ATOM | 1167 | C | ASP | A | 169 | 36.529 | 60.520 | −2.035 | 1.00 | 7.30 | A |
| ATOM | 1168 | O | ASP | A | 169 | 37.622 | 60.171 | −2.484 | 1.00 | 7.72 | A |
| ATOM | 1169 | N | GLU | A | 170 | 35.397 | 59.917 | −2.367 | 1.00 | 7.11 | A |
| ATOM | 1170 | CA | GLU | A | 170 | 35.402 | 58.807 | −3.308 | 1.00 | 7.03 | A |
| ATOM | 1171 | CB | GLU | A | 170 | 34.793 | 57.568 | −2.635 | 1.00 | 7.28 | A |
| ATOM | 1172 | CG | GLU | A | 170 | 35.628 | 57.106 | −1.433 | 1.00 | 6.70 | A |
| ATOM | 1173 | CD | GLU | A | 170 | 35.087 | 55.863 | −0.743 | 1.00 | 8.50 | A |
| ATOM | 1174 | OE1 | GLU | A | 170 | 34.335 | 55.098 | −1.377 | 1.00 | 8.21 | A |
| ATOM | 1175 | OE2 | GLU | A | 170 | 35.445 | 55.640 | 0.435 | 1.00 | 10.30 | A |
| ATOM | 1176 | C | GLU | A | 170 | 34.678 | 59.144 | −4.607 | 1.00 | 6.58 | A |
| ATOM | 1177 | O | GLU | A | 170 | 34.651 | 58.338 | −5.532 | 1.00 | 6.89 | A |
| ATOM | 1178 | N | ALA | A | 171 | 34.116 | 60.348 | −4.689 | 1.00 | 6.82 | A |
| ATOM | 1179 | CA | ALA | A | 171 | 33.395 | 60.760 | −5.887 | 1.00 | 7.07 | A |
| ATOM | 1180 | CB | ALA | A | 171 | 32.181 | 61.600 | −5.496 | 1.00 | 7.61 | A |
| ATOM | 1181 | C | ALA | A | 171 | 34.235 | 61.531 | −6.900 | 1.00 | 7.36 | A |
| ATOM | 1182 | O | ALA | A | 171 | 34.266 | 61.190 | −8.077 | 1.00 | 7.95 | A |
| ATOM | 1183 | N | ASN | A | 172 | 34.919 | 62.568 | −6.430 | 1.00 | 7.26 | A |
| ATOM | 1184 | CA | ASN | A | 172 | 35.711 | 63.440 | −7.293 | 1.00 | 7.01 | A |
| ATOM | 1185 | CB | ASN | A | 172 | 35.647 | 64.866 | −6.747 | 1.00 | 6.83 | A |
| ATOM | 1186 | CG | ASN | A | 172 | 34.230 | 65.390 | −6.639 | 1.00 | 8.36 | A |
| ATOM | 1187 | OD1 | ASN | A | 172 | 33.547 | 65.570 | −7.645 | 1.00 | 10.35 | A |
| ATOM | 1188 | ND2 | ASN | A | 172 | 33.784 | 65.645 | −5.413 | 1.00 | 8.74 | A |
| ATOM | 1189 | C | ASN | A | 172 | 37.176 | 63.059 | −7.444 | 1.00 | 6.72 | A |
| ATOM | 1190 | O | ASN | A | 172 | 37.838 | 63.476 | −8.388 | 1.00 | 6.28 | A |
| ATOM | 1191 | N | SER | A | 173 | 37.678 | 62.276 | −6.505 | 1.00 | 6.15 | A |
| ATOM | 1192 | CA | SER | A | 173 | 39.078 | 61.878 | −6.510 | 1.00 | 5.69 | A |
| ATOM | 1193 | CB | SER | A | 173 | 39.411 | 61.182 | −5.192 | 1.00 | 5.95 | A |
| ATOM | 1194 | OG | SER | A | 173 | 38.522 | 60.099 | −4.972 | 1.00 | 7.26 | A |
| ATOM | 1195 | C | SER | A | 173 | 39.470 | 60.970 | −7.661 | 1.00 | 5.64 | A |
| ATOM | 1196 | O | SER | A | 173 | 38.714 | 60.081 | −8.057 | 1.00 | 6.47 | A |
| ATOM | 1197 | N | HIS | A | 174 | 40.660 | 61.201 | −8.206 | 1.00 | 5.59 | A |
| ATOM | 1198 | CA | HIS | A | 174 | 41.154 | 60.349 | −9.276 | 1.00 | 5.24 | A |
| ATOM | 1199 | CB | HIS | A | 174 | 42.168 | 61.096 | −10.130 | 1.00 | 5.98 | A |
| ATOM | 1200 | CG | HIS | A | 174 | 42.448 | 60.421 | −11.429 | 1.00 | 6.54 | A |
| ATOM | 1201 | CD2 | HIS | A | 174 | 42.041 | 60.711 | −12.686 | 1.00 | 6.89 | A |
| ATOM | 1202 | ND1 | HIS | A | 174 | 43.161 | 59.244 | −11.516 | 1.00 | 7.16 | A |
| ATOM | 1203 | CE1 | HIS | A | 174 | 43.175 | 58.837 | −12.772 | 1.00 | 7.66 | A |
| ATOM | 1204 | NE2 | HIS | A | 174 | 42.502 | 59.709 | −13.501 | 1.00 | 8.19 | A |
| ATOM | 1205 | C | HIS | A | 174 | 41.819 | 59.155 | −8.587 | 1.00 | 5.74 | A |
| ATOM | 1206 | O | HIS | A | 174 | 42.465 | 59.321 | −7.553 | 1.00 | 5.68 | A |
| ATOM | 1207 | N | TRP | A | 175 | 41.669 | 57.956 | −9.141 | 1.00 | 4.96 | A |
| ATOM | 1208 | CA | TRP | A | 175 | 42.259 | 56.788 | −8.496 | 1.00 | 5.44 | A |
| ATOM | 1209 | CB | TRP | A | 175 | 41.995 | 55.505 | −9.301 | 1.00 | 5.91 | A |
| ATOM | 1210 | CG | TRP | A | 175 | 42.826 | 55.326 | −10.548 | 1.00 | 5.89 | A |
| ATOM | 1211 | CD2 | TRP | A | 175 | 44.114 | 54.702 | −10.634 | 1.00 | 6.41 | A |
| ATOM | 1212 | CE2 | TRP | A | 175 | 44.502 | 54.723 | −11.992 | 1.00 | 6.86 | A |
| ATOM | 1213 | CE3 | TRP | A | 175 | 44.979 | 54.125 | −9.691 | 1.00 | 6.58 | A |
| ATOM | 1214 | CD1 | TRP | A | 175 | 42.493 | 55.694 | −11.823 | 1.00 | 5.81 | A |
| ATOM | 1215 | NE1 | TRP | A | 175 | 43.495 | 55.332 | −12.697 | 1.00 | 6.79 | A |
| ATOM | 1216 | CZ2 | TRP | A | 175 | 45.721 | 54.189 | −12.434 | 1.00 | 6.92 | A |
| ATOM | 1217 | CZ3 | TRP | A | 175 | 46.190 | 53.594 | −10.129 | 1.00 | 7.55 | A |
| ATOM | 1218 | CH2 | TRP | A | 175 | 46.548 | 53.631 | −11.491 | 1.00 | 7.43 | A |
| ATOM | 1219 | C | TRP | A | 175 | 43.751 | 56.954 | −8.260 | 1.00 | 6.06 | A |
| ATOM | 1220 | O | TRP | A | 175 | 44.277 | 56.456 | −7.272 | 1.00 | 6.00 | A |
| ATOM | 1221 | N | ARG | A | 176 | 44.434 | 57.665 | −9.152 | 1.00 | 6.01 | A |
| ATOM | 1222 | CA | ARG | A | 176 | 45.868 | 57.866 | −8.983 | 1.00 | 6.21 | A |
| ATOM | 1223 | CB | ARG | A | 176 | 46.434 | 58.611 | −10.195 | 1.00 | 7.10 | A |
| ATOM | 1224 | CG | ARG | A | 176 | 46.488 | 57.732 | −11.429 | 1.00 | 7.74 | A |
| ATOM | 1225 | CD | ARG | A | 176 | 46.454 | 58.549 | −12.698 | 1.00 | 9.22 | A |
| ATOM | 1226 | NE | ARG | A | 176 | 47.557 | 59.491 | −12.790 | 1.00 | 9.75 | A |
| ATOM | 1227 | CZ | ARG | A | 176 | 47.708 | 60.339 | −13.801 | 1.00 | 10.35 | A |
| ATOM | 1228 | NH1 | ARG | A | 176 | 46.825 | 60.348 | −14.791 | 1.00 | 10.58 | A |
| ATOM | 1229 | NH2 | ARG | A | 176 | 48.729 | 61.182 | −13.817 | 1.00 | 11.03 | A |
| ATOM | 1230 | C | ARG | A | 176 | 46.178 | 58.617 | −7.690 | 1.00 | 5.98 | A |
| ATOM | 1231 | O | ARG | A | 176 | 47.167 | 58.317 | −7.011 | 1.00 | 7.04 | A |
| ATOM | 1232 | N | ASN | A | 177 | 45.329 | 59.579 | −7.335 | 1.00 | 5.86 | A |
| ATOM | 1233 | CA | ASN | A | 177 | 45.551 | 60.343 | −6.110 | 1.00 | 5.50 | A |
| ATOM | 1234 | CB | ASN | A | 177 | 44.900 | 61.723 | −6.208 | 1.00 | 6.39 | A |
| ATOM | 1235 | CG | ASN | A | 177 | 45.571 | 62.590 | −7.241 | 1.00 | 8.49 | A |
| ATOM | 1236 | OD1 | ASN | A | 177 | 46.756 | 62.416 | −7.528 | 1.00 | 7.81 | A |
| ATOM | 1237 | ND2 | ASN | A | 177 | 44.825 | 63.533 | −7.806 | 1.00 | 9.47 | A |
| ATOM | 1238 | C | ASN | A | 177 | 45.065 | 59.598 | −4.877 | 1.00 | 5.82 | A |
| ATOM | 1239 | O | ASN | A | 177 | 45.568 | 59.814 | −3.768 | 1.00 | 5.55 | A |
| ATOM | 1240 | N | VAL | A | 178 | 44.086 | 58.721 | −5.059 | 1.00 | 5.11 | A |
| ATOM | 1241 | CA | VAL | A | 178 | 43.619 | 57.922 | −3.934 | 1.00 | 5.43 | A |
| ATOM | 1242 | CB | VAL | A | 178 | 42.405 | 57.054 | −4.310 | 1.00 | 5.87 | A |
| ATOM | 1243 | CG1 | VAL | A | 178 | 42.125 | 56.045 | −3.199 | 1.00 | 6.99 | A |
| ATOM | 1244 | CG2 | VAL | A | 178 | 41.189 | 57.933 | −4.523 | 1.00 | 7.19 | A |
| ATOM | 1245 | C | VAL | A | 178 | 44.794 | 57.013 | −3.570 | 1.00 | 5.88 | A |
| ATOM | 1246 | O | VAL | A | 178 | 45.102 | 56.816 | −2.396 | 1.00 | 6.98 | A |
| ATOM | 1247 | N | LEU | A | 179 | 45.463 | 56.469 | −4.581 | 1.00 | 6.29 | A |
| ATOM | 1248 | CA | LEU | A | 179 | 46.609 | 55.606 | −4.321 | 1.00 | 6.10 | A |
| ATOM | 1249 | CB | LEU | A | 179 | 47.081 | 54.914 | −5.610 | 1.00 | 6.48 | A |
| ATOM | 1250 | CG | LEU | A | 179 | 48.388 | 54.104 | −5.494 | 1.00 | 6.83 | A |
| ATOM | 1251 | CD1 | LEU | A | 179 | 48.249 | 52.970 | −4.471 | 1.00 | 7.54 | A |
| ATOM | 1252 | CD2 | LEU | A | 179 | 48.737 | 53.534 | −6.862 | 1.00 | 7.81 | A |
| ATOM | 1253 | C | LEU | A | 179 | 47.760 | 56.409 | −3.720 | 1.00 | 6.01 | A |
| ATOM | 1254 | O | LEU | A | 179 | 48.436 | 55.947 | −2.801 | 1.00 | 5.97 | A |
| ATOM | 1255 | N | LEU | A | 180 | 47.971 | 57.619 | −4.234 | 1.00 | 5.99 | A |
| ATOM | 1256 | CA | LEU | A | 180 | 49.047 | 58.476 | −3.751 | 1.00 | 5.46 | A |
| ATOM | 1257 | CB | LEU | A | 180 | 49.034 | 59.816 | −4.493 | 1.00 | 6.21 | A |
| ATOM | 1258 | CG | LEU | A | 180 | 50.171 | 60.777 | −4.137 | 1.00 | 8.11 | A |
| ATOM | 1259 | CD1 | LEU | A | 180 | 51.464 | 60.276 | −4.762 | 1.00 | 8.40 | A |
| ATOM | 1260 | CD2 | LEU | A | 180 | 49.844 | 62.174 | −4.644 | 1.00 | 8.88 | A |
| ATOM | 1261 | C | LEU | A | 180 | 48.917 | 58.733 | −2.259 | 1.00 | 5.49 | A |
| ATOM | 1262 | O | LEU | A | 180 | 49.873 | 58.539 | −1.504 | 1.00 | 6.38 | A |
| ATOM | 1263 | N | GLN | A | 181 | 47.732 | 59.147 | −1.823 | 1.00 | 5.36 | A |
| ATOM | 1264 | CA | GLN | A | 181 | 47.558 | 59.450 | −0.408 | 1.00 | 6.03 | A |
| ATOM | 1265 | CB | GLN | A | 181 | 46.263 | 60.243 | −0.177 | 1.00 | 6.02 | A |
| ATOM | 1266 | CG | GLN | A | 181 | 44.963 | 59.508 | −0.445 | 1.00 | 7.15 | A |
| ATOM | 1267 | CD | GLN | A | 181 | 44.584 | 58.572 | 0.681 | 1.00 | 6.82 | A |
| ATOM | 1268 | OE1 | GLN | A | 181 | 44.809 | 58.871 | 1.858 | 1.00 | 7.67 | A |
| ATOM | 1269 | NE2 | GLN | A | 181 | 43.983 | 57.445 | 0.330 | 1.00 | 8.04 | A |
| ATOM | 1270 | C | GLN | A | 181 | 47.616 | 58.203 | 0.466 | 1.00 | 5.96 | A |
| ATOM | 1271 | O | GLN | A | 181 | 48.110 | 58.257 | 1.593 | 1.00 | 6.48 | A |
| ATOM | 1272 | N | LEU | A | 182 | 47.137 | 57.073 | −0.052 | 1.00 | 5.92 | A |
| ATOM | 1273 | CA | LEU | A | 182 | 47.190 | 55.826 | 0.709 | 1.00 | 5.60 | A |
| ATOM | 1274 | CB | LEU | A | 182 | 46.467 | 54.701 | −0.037 | 1.00 | 6.36 | A |
| ATOM | 1275 | CG | LEU | A | 182 | 46.529 | 53.321 | 0.626 | 1.00 | 6.42 | A |
| ATOM | 1276 | CD1 | LEU | A | 182 | 45.774 | 53.341 | 1.957 | 1.00 | 7.96 | A |
| ATOM | 1277 | CD2 | LEU | A | 182 | 45.924 | 52.275 | −0.313 | 1.00 | 7.27 | A |
| ATOM | 1278 | C | LEU | A | 182 | 48.654 | 55.448 | 0.900 | 1.00 | 6.22 | A |
| ATOM | 1279 | O | LEU | A | 182 | 49.071 | 55.048 | 1.986 | 1.00 | 6.59 | A |
| ATOM | 1280 | N | THR | A | 183 | 49.437 | 55.590 | −0.163 | 1.00 | 6.40 | A |
| ATOM | 1281 | CA | THR | A | 183 | 50.854 | 55.254 | −0.103 | 1.00 | 6.70 | A |
| ATOM | 1282 | CB | THR | A | 183 | 51.488 | 55.323 | −1.510 | 1.00 | 7.61 | A |
| ATOM | 1283 | OG1 | THR | A | 183 | 50.795 | 54.429 | −2.392 | 1.00 | 6.66 | A |
| ATOM | 1284 | CG2 | THR | A | 183 | 52.948 | 54.924 | −1.453 | 1.00 | 8.69 | A |
| ATOM | 1285 | C | THR | A | 183 | 51.601 | 56.197 | 0.843 | 1.00 | 6.95 | A |
| ATOM | 1286 | O | THR | A | 183 | 52.477 | 55.773 | 1.594 | 1.00 | 6.77 | A |
| ATOM | 1287 | N | GLU | A | 184 | 51.248 | 57.477 | 0.815 | 1.00 | 6.54 | A |
| ATOM | 1288 | CA | GLU | A | 184 | 51.907 | 58.454 | 1.678 | 1.00 | 7.23 | A |
| ATOM | 1289 | CB | GLU | A | 184 | 51.345 | 59.852 | 1.399 | 1.00 | 8.96 | A |
| ATOM | 1290 | CG | GLU | A | 184 | 52.194 | 61.013 | 1.919 | 1.00 | 10.85 | A |
| ATOM | 1291 | CD | GLU | A | 184 | 53.598 | 61.053 | 1.324 | 1.00 | 11.51 | A |
| ATOM | 1292 | OE1 | GLU | A | 184 | 53.778 | 60.668 | 0.149 | 1.00 | 13.38 | A |
| ATOM | 1293 | OE2 | GLU | A | 184 | 54.523 | 61.490 | 2.037 | 1.00 | 14.02 | A |
| ATOM | 1294 | C | GLU | A | 184 | 51.704 | 58.082 | 3.148 | 1.00 | 7.89 | A |
| ATOM | 1295 | O | GLU | A | 184 | 52.651 | 58.072 | 3.938 | 1.00 | 8.95 | A |
| ATOM | 1296 | N | GLY | A | 185 | 50.465 | 57.767 | 3.506 | 1.00 | 7.49 | A |
| ATOM | 1297 | CA | GLY | A | 185 | 50.165 | 57.395 | 4.876 | 1.00 | 7.51 | A |
| ATOM | 1298 | C | GLY | A | 185 | 50.754 | 56.056 | 5.292 | 1.00 | 7.61 | A |
| ATOM | 1299 | O | GLY | A | 185 | 51.324 | 55.934 | 6.379 | 1.00 | 7.79 | A |
| ATOM | 1300 | N | GLN | A | 186 | 50.638 | 55.045 | 4.437 | 1.00 | 7.55 | A |
| ATOM | 1301 | CA | GLN | A | 186 | 51.154 | 53.732 | 4.803 | 1.00 | 7.94 | A |
| ATOM | 1302 | CB | GLN | A | 186 | 50.573 | 52.653 | 3.890 | 1.00 | 7.79 | A |
| ATOM | 1303 | CG | GLN | A | 186 | 49.075 | 52.469 | 4.067 | 1.00 | 9.35 | A |
| ATOM | 1304 | CD | GLN | A | 186 | 48.647 | 51.045 | 3.819 | 1.00 | 10.10 | A |
| ATOM | 1305 | OE1 | GLN | A | 186 | 49.239 | 50.349 | 2.999 | 1.00 | 12.28 | A |
| ATOM | 1306 | NE2 | GLN | A | 186 | 47.615 | 50.598 | 4.525 | 1.00 | 9.16 | A |
| ATOM | 1307 | C | GLN | A | 186 | 52.670 | 53.652 | 4.802 | 1.00 | 8.34 | A |
| ATOM | 1308 | O | GLN | A | 186 | 53.251 | 52.893 | 5.575 | 1.00 | 7.45 | A |
| ATOM | 1309 | N | THR | A | 187 | 53.322 | 54.417 | 3.937 | 1.00 | 8.73 | A |
| ATOM | 1310 | CA | THR | A | 187 | 54.776 | 54.393 | 3.914 | 1.00 | 8.31 | A |
| ATOM | 1311 | CB | THR | A | 187 | 55.313 | 55.199 | 2.723 | 1.00 | 8.96 | A |
| ATOM | 1312 | OG1 | THR | A | 187 | 54.836 | 54.603 | 1.510 | 1.00 | 8.94 | A |
| ATOM | 1313 | CG2 | THR | A | 187 | 56.839 | 55.194 | 2.709 | 1.00 | 9.08 | A |
| ATOM | 1314 | C | THR | A | 187 | 55.280 | 54.966 | 5.239 | 1.00 | 8.89 | A |
| ATOM | 1315 | O | THR | A | 187 | 56.236 | 54.451 | 5.826 | 1.00 | 8.76 | A |
| ATOM | 1316 | N | TRP | A | 188 | 54.620 | 56.016 | 5.720 | 1.00 | 8.63 | A |
| ATOM | 1317 | CA | TRP | A | 188 | 54.992 | 56.628 | 6.989 | 1.00 | 8.92 | A |
| ATOM | 1318 | CB | TRP | A | 188 | 54.131 | 57.863 | 7.265 | 1.00 | 9.67 | A |
| ATOM | 1319 | CG | TRP | A | 188 | 54.583 | 58.643 | 8.464 | 1.00 | 9.58 | A |
| ATOM | 1320 | CD2 | TRP | A | 188 | 54.248 | 58.383 | 9.834 | 1.00 | 9.95 | A |
| ATOM | 1321 | CE2 | TRP | A | 188 | 54.973 | 59.304 | 10.622 | 1.00 | 10.10 | A |
| ATOM | 1322 | CE3 | TRP | A | 188 | 53.409 | 57.458 | 10.472 | 1.00 | 9.95 | A |
| ATOM | 1323 | CD1 | TRP | A | 188 | 55.466 | 59.685 | 8.477 | 1.00 | 10.29 | A |
| ATOM | 1324 | NE1 | TRP | A | 188 | 55.708 | 60.086 | 9.770 | 1.00 | 10.60 | A |
| ATOM | 1325 | CZ2 | TRP | A | 188 | 54.888 | 59.328 | 12.018 | 1.00 | 9.73 | A |
| ATOM | 1326 | CZ3 | TRP | A | 188 | 53.325 | 57.480 | 11.866 | 1.00 | 10.52 | A |
| ATOM | 1327 | CH2 | TRP | A | 188 | 54.062 | 58.411 | 12.621 | 1.00 | 10.61 | A |
| ATOM | 1328 | C | TRP | A | 188 | 54.773 | 55.604 | 8.104 | 1.00 | 8.69 | A |
| ATOM | 1329 | O | TRP | A | 188 | 55.635 | 55.399 | 8.958 | 1.00 | 8.79 | A |
| ATOM | 1330 | N | LEU | A | 189 | 53.614 | 54.955 | 8.097 | 1.00 | 8.25 | A |
| ATOM | 1331 | CA | LEU | A | 189 | 53.323 | 53.962 | 9.126 | 1.00 | 9.12 | A |
| ATOM | 1332 | CB | LEU | A | 189 | 51.918 | 53.387 | 8.936 | 1.00 | 8.95 | A |
| ATOM | 1333 | CG | LEU | A | 189 | 50.767 | 54.284 | 9.391 | 1.00 | 8.34 | A |
| ATOM | 1334 | CD1 | LEU | A | 189 | 49.438 | 53.610 | 9.072 | 1.00 | 9.27 | A |
| ATOM | 1335 | CD2 | LEU | A | 189 | 50.883 | 54.545 | 10.894 | 1.00 | 9.17 | A |
| ATOM | 1336 | C | LEU | A | 189 | 54.332 | 52.820 | 9.155 | 1.00 | 9.47 | A |
| ATOM | 1337 | O | LEU | A | 189 | 54.736 | 52.371 | 10.229 | 1.00 | 10.32 | A |
| ATOM | 1338 | N | LYS | A | 190 | 54.743 | 52.340 | 7.989 | 1.00 | 8.83 | A |
| ATOM | 1339 | CA | LYS | A | 190 | 55.699 | 51.240 | 7.965 | 1.00 | 10.73 | A |
| ATOM | 1340 | CB | LYS | A | 190 | 55.904 | 50.721 | 6.538 | 1.00 | 11.97 | A |
| ATOM | 1341 | CG | LYS | A | 190 | 56.763 | 49.460 | 6.471 | 1.00 | 14.62 | A |
| ATOM | 1342 | CD | LYS | A | 190 | 56.884 | 48.941 | 5.050 | 1.00 | 18.07 | A |
| ATOM | 1343 | CE | LYS | A | 190 | 57.597 | 47.600 | 5.015 | 1.00 | 20.94 | A |
| ATOM | 1344 | NZ | LYS | A | 190 | 58.942 | 47.678 | 5.649 | 1.00 | 22.98 | A |
| ATOM | 1345 | C | LYS | A | 190 | 57.037 | 51.682 | 8.543 | 1.00 | 11.26 | A |
| ATOM | 1346 | O | LYS | A | 190 | 57.650 | 50.964 | 9.333 | 1.00 | 11.22 | A |
| ATOM | 1347 | N | GLN | A | 191 | 57.479 | 52.872 | 8.157 | 1.00 | 11.65 | A |
| ATOM | 1348 | CA | GLN | A | 191 | 58.753 | 53.396 | 8.625 | 1.00 | 14.13 | A |
| ATOM | 1349 | CB | GLN | A | 191 | 59.132 | 54.655 | 7.836 | 1.00 | 15.24 | A |
| ATOM | 1350 | CG | GLN | A | 191 | 60.462 | 55.263 | 8.265 | 1.00 | 19.98 | A |
| ATOM | 1351 | CD | GLN | A | 191 | 60.871 | 56.459 | 7.425 | 1.00 | 21.27 | A |
| ATOM | 1352 | OE1 | GLN | A | 191 | 61.906 | 57.079 | 7.675 | 1.00 | 24.60 | A |
| ATOM | 1353 | NE2 | GLN | A | 191 | 60.063 | 56.789 | 6.422 | 1.00 | 22.87 | A |
| ATOM | 1354 | C | GLN | A | 191 | 58.798 | 53.711 | 10.116 | 1.00 | 14.03 | A |
| ATOM | 1355 | O | GLN | A | 191 | 59.758 | 53.345 | 10.797 | 1.00 | 16.11 | A |
| ATOM | 1356 | N | PHE | A | 192 | 57.766 | 54.372 | 10.631 | 1.00 | 13.64 | A |
| ATOM | 1357 | CA | PHE | A | 192 | 57.760 | 54.756 | 12.040 | 1.00 | 13.50 | A |
| ATOM | 1358 | CB | PHE | A | 192 | 57.333 | 56.220 | 12.166 | 1.00 | 13.17 | A |
| ATOM | 1359 | CG | PHE | A | 192 | 58.233 | 57.171 | 11.433 | 1.00 | 13.74 | A |
| ATOM | 1360 | CD1 | PHE | A | 192 | 57.918 | 57.600 | 10.147 | 1.00 | 13.68 | A |
| ATOM | 1361 | CD2 | PHE | A | 192 | 59.418 | 57.611 | 12.014 | 1.00 | 14.33 | A |
| ATOM | 1362 | CE1 | PHE | A | 192 | 58.768 | 58.453 | 9.449 | 1.00 | 13.88 | A |
| ATOM | 1363 | CE2 | PHE | A | 192 | 60.278 | 58.466 | 11.322 | 1.00 | 14.18 | A |
| ATOM | 1364 | CZ | PHE | A | 192 | 59.952 | 58.887 | 10.040 | 1.00 | 14.41 | A |
| ATOM | 1365 | C | PHE | A | 192 | 56.971 | 53.908 | 13.040 | 1.00 | 14.01 | A |
| ATOM | 1366 | O | PHE | A | 192 | 57.338 | 53.864 | 14.216 | 1.00 | 15.20 | A |
| ATOM | 1367 | N | MSE | A | 193 | 55.901 | 53.248 | 12.598 | 1.00 | 12.52 | A |
| ATOM | 1368 | CA | MSE | A | 193 | 55.093 | 52.408 | 13.496 | 1.00 | 13.30 | A |
| ATOM | 1369 | CB | MSE | A | 193 | 53.587 | 52.680 | 13.337 | 1.00 | 15.39 | A |
| ATOM | 1370 | CG | MSE | A | 193 | 53.007 | 53.917 | 14.029 | 1.00 | 17.42 | A |
| ATOM | 1371 | SE | MSE | A | 193 | 53.504 | 54.201 | 15.886 | 1.00 | 27.71 | A |
| ATOM | 1372 | CE | MSE | A | 193 | 54.844 | 55.450 | 15.340 | 1.00 | 9.34 | A |
| ATOM | 1373 | C | MSE | A | 193 | 55.312 | 50.919 | 13.235 | 1.00 | 13.10 | A |
| ATOM | 1374 | O | MSE | A | 193 | 54.889 | 50.077 | 14.027 | 1.00 | 13.59 | A |
| ATOM | 1375 | N | ASN | A | 194 | 55.954 | 50.599 | 12.118 | 1.00 | 12.20 | A |
| ATOM | 1376 | CA | ASN | A | 194 | 56.204 | 49.212 | 11.739 | 1.00 | 12.23 | A |
| ATOM | 1377 | CB | ASN | A | 194 | 57.146 | 48.540 | 12.747 | 1.00 | 13.82 | A |
| ATOM | 1378 | CG | ASN | A | 194 | 57.600 | 47.163 | 12.295 | 1.00 | 15.07 | A |
| ATOM | 1379 | OD1 | ASN | A | 194 | 57.775 | 46.920 | 11.099 | 1.00 | 16.05 | A |
| ATOM | 1380 | ND2 | ASN | A | 194 | 57.804 | 46.268 | 13.258 | 1.00 | 16.16 | A |
| ATOM | 1381 | C | ASN | A | 194 | 54.902 | 48.416 | 11.628 | 1.00 | 12.35 | A |
| ATOM | 1382 | O | ASN | A | 194 | 54.803 | 47.287 | 12.114 | 1.00 | 12.62 | A |
| ATOM | 1383 | N | VAL | A | 195 | 53.899 | 49.017 | 10.998 | 1.00 | 11.62 | A |
| ATOM | 1384 | CA | VAL | A | 195 | 52.616 | 48.353 | 10.806 | 1.00 | 12.01 | A |
| ATOM | 1385 | CB | VAL | A | 195 | 51.599 | 48.684 | 11.937 | 1.00 | 12.67 | A |
| ATOM | 1386 | CG1 | VAL | A | 195 | 52.148 | 48.252 | 13.288 | 1.00 | 14.48 | A |
| ATOM | 1387 | CG2 | VAL | A | 195 | 51.268 | 50.165 | 11.935 | 1.00 | 12.78 | A |
| ATOM | 1388 | C | VAL | A | 195 | 52.000 | 48.793 | 9.486 | 1.00 | 11.41 | A |
| ATOM | 1389 | O | VAL | A | 195 | 52.227 | 49.914 | 9.029 | 1.00 | 10.81 | A |
| ATOM | 1390 | N | THR | A | 196 | 51.230 | 47.894 | 8.882 | 1.00 | 10.57 | A |
| ATOM | 1391 | CA | THR | A | 196 | 50.536 | 48.171 | 7.627 | 1.00 | 10.52 | A |
| ATOM | 1392 | CB | THR | A | 196 | 51.191 | 47.438 | 6.429 | 1.00 | 10.51 | A |
| ATOM | 1393 | OG1 | THR | A | 196 | 52.554 | 47.862 | 6.288 | 1.00 | 11.89 | A |
| ATOM | 1394 | CG2 | THR | A | 196 | 50.440 | 47.753 | 5.143 | 1.00 | 11.97 | A |
| ATOM | 1395 | C | THR | A | 196 | 49.096 | 47.680 | 7.785 | 1.00 | 10.00 | A |
| ATOM | 1396 | O | THR | A | 196 | 48.838 | 46.476 | 7.812 | 1.00 | 10.58 | A |
| ATOM | 1397 | N | PRO | A | 197 | 48.137 | 48.610 | 7.903 | 1.00 | 9.62 | A |
| ATOM | 1398 | CD | PRO | A | 197 | 48.314 | 50.068 | 8.022 | 1.00 | 9.15 | A |
| ATOM | 1399 | CA | PRO | A | 197 | 46.727 | 48.245 | 8.060 | 1.00 | 9.26 | A |
| ATOM | 1400 | CB | PRO | A | 197 | 46.024 | 49.600 | 8.096 | 1.00 | 9.55 | A |
| ATOM | 1401 | CG | PRO | A | 197 | 47.040 | 50.487 | 8.717 | 1.00 | 9.10 | A |
| ATOM | 1402 | C | PRO | A | 197 | 46.192 | 47.375 | 6.929 | 1.00 | 9.50 | A |
| ATOM | 1403 | O | PRO | A | 197 | 46.534 | 47.579 | 5.762 | 1.00 | 9.51 | A |
| ATOM | 1404 | N | THR | A | 198 | 45.355 | 46.404 | 7.279 | 1.00 | 9.16 | A |
| ATOM | 1405 | CA | THR | A | 198 | 44.743 | 45.540 | 6.278 | 1.00 | 9.69 | A |
| ATOM | 1406 | CB | THR | A | 198 | 45.125 | 44.060 | 6.466 | 1.00 | 10.81 | A |
| ATOM | 1407 | OG1 | THR | A | 198 | 44.660 | 43.600 | 7.739 | 1.00 | 11.74 | A |
| ATOM | 1408 | CG2 | THR | A | 198 | 46.635 | 43.887 | 6.368 | 1.00 | 11.84 | A |
| ATOM | 1409 | C | THR | A | 198 | 43.229 | 45.682 | 6.363 | 1.00 | 8.80 | A |
| ATOM | 1410 | O | THR | A | 198 | 42.491 | 44.986 | 5.671 | 1.00 | 8.81 | A |
| ATOM | 1411 | N | ALA | A | 199 | 42.776 | 46.586 | 7.227 | 1.00 | 8.53 | A |
| ATOM | 1412 | CA | ALA | A | 199 | 41.353 | 46.865 | 7.387 | 1.00 | 8.67 | A |
| ATOM | 1413 | CB | ALA | A | 199 | 40.887 | 46.477 | 8.790 | 1.00 | 9.80 | A |
| ATOM | 1414 | C | ALA | A | 199 | 41.146 | 48.360 | 7.157 | 1.00 | 8.79 | A |
| ATOM | 1415 | O | ALA | A | 199 | 41.870 | 49.182 | 7.721 | 1.00 | 8.61 | A |
| ATOM | 1416 | N | SER | A | 200 | 40.162 | 48.707 | 6.332 | 1.00 | 8.10 | A |
| ATOM | 1417 | CA | SER | A | 200 | 39.873 | 50.102 | 6.023 | 1.00 | 8.52 | A |
| ATOM | 1418 | CB | SER | A | 200 | 39.724 | 50.280 | 4.511 | 1.00 | 8.51 | A |
| ATOM | 1419 | OG | SER | A | 200 | 39.498 | 51.638 | 4.174 | 1.00 | 10.82 | A |
| ATOM | 1420 | C | SER | A | 200 | 38.620 | 50.603 | 6.740 | 1.00 | 8.16 | A |
| ATOM | 1421 | O | SER | A | 200 | 37.663 | 49.854 | 6.951 | 1.00 | 8.18 | A |
| ATOM | 1422 | N | TRP | A | 201 | 38.646 | 51.882 | 7.101 | 1.00 | 8.42 | A |
| ATOM | 1423 | CA | TRP | A | 201 | 37.563 | 52.539 | 7.827 | 1.00 | 8.34 | A |
| ATOM | 1424 | CB | TRP | A | 201 | 38.057 | 52.782 | 9.263 | 1.00 | 8.58 | A |
| ATOM | 1425 | CG | TRP | A | 201 | 37.224 | 53.618 | 10.202 | 1.00 | 8.17 | A |
| ATOM | 1426 | CD2 | TRP | A | 201 | 36.231 | 53.144 | 11.123 | 1.00 | 9.44 | A |
| ATOM | 1427 | CE2 | TRP | A | 201 | 35.838 | 54.245 | 11.919 | 1.00 | 9.61 | A |
| ATOM | 1428 | CE3 | TRP | A | 201 | 35.641 | 51.896 | 11.359 | 1.00 | 10.07 | A |
| ATOM | 1429 | CD1 | TRP | A | 201 | 37.375 | 54.950 | 10.461 | 1.00 | 9.35 | A |
| ATOM | 1430 | NE1 | TRP | A | 201 | 36.552 | 55.333 | 11.492 | 1.00 | 9.72 | A |
| ATOM | 1431 | CZ2 | TRP | A | 201 | 34.882 | 54.135 | 12.935 | 1.00 | 9.92 | A |
| ATOM | 1432 | CZ3 | TRP | A | 201 | 34.688 | 51.784 | 12.372 | 1.00 | 10.54 | A |
| ATOM | 1433 | CH2 | TRP | A | 201 | 34.321 | 52.900 | 13.147 | 1.00 | 10.12 | A |
| ATOM | 1434 | C | TRP | A | 201 | 37.220 | 53.844 | 7.111 | 1.00 | 8.73 | A |
| ATOM | 1435 | O | TRP | A | 201 | 37.995 | 54.796 | 7.144 | 1.00 | 9.70 | A |
| ATOM | 1436 | N | ALA | A | 202 | 36.066 | 53.873 | 6.447 | 1.00 | 8.52 | A |
| ATOM | 1437 | CA | ALA | A | 202 | 35.617 | 55.062 | 5.716 | 1.00 | 8.40 | A |
| ATOM | 1438 | CB | ALA | A | 202 | 35.678 | 54.808 | 4.211 | 1.00 | 9.54 | A |
| ATOM | 1439 | C | ALA | A | 202 | 34.192 | 55.400 | 6.144 | 1.00 | 8.89 | A |
| ATOM | 1440 | O | ALA | A | 202 | 33.220 | 55.018 | 5.495 | 1.00 | 8.55 | A |
| ATOM | 1441 | N | ILE | A | 203 | 34.082 | 56.134 | 7.242 | 1.00 | 8.07 | A |
| ATOM | 1442 | CA | ILE | A | 203 | 32.785 | 56.484 | 7.800 | 1.00 | 8.51 | A |
| ATOM | 1443 | CB | ILE | A | 203 | 32.842 | 56.503 | 9.354 | 1.00 | 8.05 | A |
| ATOM | 1444 | CG2 | ILE | A | 203 | 33.116 | 55.091 | 9.885 | 1.00 | 8.11 | A |
| ATOM | 1445 | CG1 | ILE | A | 203 | 33.907 | 57.500 | 9.832 | 1.00 | 9.39 | A |
| ATOM | 1446 | CD1 | ILE | A | 203 | 33.900 | 57.737 | 11.341 | 1.00 | 8.65 | A |
| ATOM | 1447 | C | ILE | A | 203 | 32.186 | 57.806 | 7.348 | 1.00 | 8.80 | A |
| ATOM | 1448 | O | ILE | A | 203 | 31.025 | 58.073 | 7.648 | 1.00 | 8.92 | A |
| ATOM | 1449 | N | ASP | A | 204 | 32.939 | 58.621 | 6.609 | 1.00 | 8.61 | A |
| ATOM | 1450 | CA | ASP | A | 204 | 32.408 | 59.923 | 6.216 | 1.00 | 9.22 | A |
| ATOM | 1451 | CB | ASP | A | 204 | 33.146 | 61.022 | 6.987 | 1.00 | 8.73 | A |
| ATOM | 1452 | CG | ASP | A | 204 | 32.240 | 62.188 | 7.356 | 1.00 | 9.80 | A |
| ATOM | 1453 | OD1 | ASP | A | 204 | 32.765 | 63.280 | 7.652 | 1.00 | 9.95 | A |
| ATOM | 1454 | OD2 | ASP | A | 204 | 31.004 | 62.019 | 7.370 | 1.00 | 10.59 | A |
| ATOM | 1455 | C | ASP | A | 204 | 32.318 | 60.338 | 4.739 | 1.00 | 9.40 | A |
| ATOM | 1456 | O | ASP | A | 204 | 31.638 | 61.318 | 4.433 | 1.00 | 10.33 | A |
| ATOM | 1457 | N | PRO | A | 205 | 33.009 | 59.641 | 3.815 | 1.00 | 9.27 | A |
| ATOM | 1458 | CD | PRO | A | 205 | 34.005 | 58.559 | 3.926 | 1.00 | 10.07 | A |
| ATOM | 1459 | CA | PRO | A | 205 | 32.872 | 60.086 | 2.418 | 1.00 | 9.44 | A |
| ATOM | 1460 | CB | PRO | A | 205 | 33.667 | 59.040 | 1.641 | 1.00 | 10.86 | A |
| ATOM | 1461 | CG | PRO | A | 205 | 34.756 | 58.679 | 2.611 | 1.00 | 11.93 | A |
| ATOM | 1462 | C | PRO | A | 205 | 31.384 | 60.090 | 2.055 | 1.00 | 9.61 | A |
| ATOM | 1463 | O | PRO | A | 205 | 30.629 | 59.244 | 2.530 | 1.00 | 10.30 | A |
| ATOM | 1464 | N | PHE | A | 206 | 30.964 | 61.032 | 1.214 | 1.00 | 9.55 | A |
| ATOM | 1465 | CA | PHE | A | 206 | 29.544 | 61.153 | 0.867 | 1.00 | 9.41 | A |
| ATOM | 1466 | CB | PHE | A | 206 | 29.220 | 62.618 | 0.553 | 1.00 | 9.27 | A |
| ATOM | 1467 | CG | PHE | A | 206 | 30.037 | 63.607 | 1.354 | 1.00 | 9.04 | A |
| ATOM | 1468 | CD1 | PHE | A | 206 | 30.301 | 63.389 | 2.705 | 1.00 | 8.94 | A |
| ATOM | 1469 | CD2 | PHE | A | 206 | 30.555 | 64.749 | 0.748 | 1.00 | 9.10 | A |
| ATOM | 1470 | CE1 | PHE | A | 206 | 31.074 | 64.294 | 3.440 | 1.00 | 8.93 | A |
| ATOM | 1471 | CE2 | PHE | A | 206 | 31.326 | 65.660 | 1.475 | 1.00 | 9.22 | A |
| ATOM | 1472 | CZ | PHE | A | 206 | 31.587 | 65.431 | 2.821 | 1.00 | 9.28 | A |
| ATOM | 1473 | C | PHE | A | 206 | 29.169 | 60.246 | −0.301 | 1.00 | 9.31 | A |
| ATOM | 1474 | O | PHE | A | 206 | 28.950 | 60.696 | −1.427 | 1.00 | 9.75 | A |
| ATOM | 1475 | N | GLY | A | 207 | 29.062 | 58.958 | −0.005 | 1.00 | 8.61 | A |
| ATOM | 1476 | CA | GLY | A | 207 | 28.775 | 57.982 | −1.037 | 1.00 | 8.81 | A |
| ATOM | 1477 | C | GLY | A | 207 | 30.072 | 57.210 | −1.191 | 1.00 | 8.17 | A |
| ATOM | 1478 | O | GLY | A | 207 | 31.137 | 57.730 | −0.858 | 1.00 | 8.29 | A |
| ATOM | 1479 | N | HIS | A | 208 | 30.001 | 55.990 | −1.712 | 1.00 | 7.51 | A |
| ATOM | 1480 | CA | HIS | A | 208 | 31.190 | 55.155 | −1.851 | 1.00 | 7.28 | A |
| ATOM | 1481 | CB | HIS | A | 208 | 31.135 | 54.027 | −0.819 | 1.00 | 7.61 | A |
| ATOM | 1482 | CG | HIS | A | 208 | 31.256 | 54.505 | 0.596 | 1.00 | 8.45 | A |
| ATOM | 1483 | CD2 | HIS | A | 208 | 30.314 | 54.797 | 1.523 | 1.00 | 8.55 | A |
| ATOM | 1484 | ND1 | HIS | A | 208 | 32.471 | 54.778 | 1.185 | 1.00 | 9.69 | A |
| ATOM | 1485 | CE1 | HIS | A | 208 | 32.273 | 55.218 | 2.415 | 1.00 | 10.15 | A |
| ATOM | 1486 | NE2 | HIS | A | 208 | 30.973 | 55.240 | 2.645 | 1.00 | 9.28 | A |
| ATOM | 1487 | C | HIS | A | 208 | 31.401 | 54.574 | −3.242 | 1.00 | 7.09 | A |
| ATOM | 1488 | O | HIS | A | 208 | 30.449 | 54.248 | −3.960 | 1.00 | 7.30 | A |
| ATOM | 1489 | N | SER | A | 209 | 32.673 | 54.429 | −3.600 | 1.00 | 6.59 | A |
| ATOM | 1490 | CA | SER | A | 209 | 33.068 | 53.925 | −4.907 | 1.00 | 6.62 | A |
| ATOM | 1491 | CB | SER | A | 209 | 33.960 | 54.960 | −5.598 | 1.00 | 6.86 | A |
| ATOM | 1492 | OG | SER | A | 209 | 34.520 | 54.434 | −6.790 | 1.00 | 6.54 | A |
| ATOM | 1493 | C | SER | A | 209 | 33.813 | 52.599 | −4.864 | 1.00 | 7.08 | A |
| ATOM | 1494 | O | SER | A | 209 | 34.622 | 52.360 | −3.972 | 1.00 | 6.40 | A |
| ATOM | 1495 | N | PRO | A | 210 | 33.548 | 51.717 | −5.841 | 1.00 | 7.03 | A |
| ATOM | 1496 | CD | PRO | A | 210 | 32.525 | 51.830 | −6.895 | 1.00 | 7.72 | A |
| ATOM | 1497 | CA | PRO | A | 210 | 34.221 | 50.415 | −5.898 | 1.00 | 7.22 | A |
| ATOM | 1498 | CB | PRO | A | 210 | 33.456 | 49.675 | −6.997 | 1.00 | 7.15 | A |
| ATOM | 1499 | CG | PRO | A | 210 | 32.977 | 50.795 | −7.889 | 1.00 | 7.88 | A |
| ATOM | 1500 | C | PRO | A | 210 | 35.712 | 50.582 | −6.211 | 1.00 | 7.12 | A |
| ATOM | 1501 | O | PRO | A | 210 | 36.481 | 49.620 | −6.181 | 1.00 | 6.74 | A |
| ATOM | 1502 | N | THR | A | 211 | 36.124 | 51.806 | −6.529 | 1.00 | 6.80 | A |
| ATOM | 1503 | CA | THR | A | 211 | 37.531 | 52.044 | −6.793 | 1.00 | 7.50 | A |
| ATOM | 1504 | CB | THR | A | 211 | 37.762 | 53.482 | −7.288 | 1.00 | 7.37 | A |
| ATOM | 1505 | OG1 | THR | A | 211 | 37.233 | 53.596 | −8.613 | 1.00 | 8.05 | A |
| ATOM | 1506 | CG2 | THR | A | 211 | 39.250 | 53.833 | −7.292 | 1.00 | 8.53 | A |
| ATOM | 1507 | C | THR | A | 211 | 38.321 | 51.800 | −5.510 | 1.00 | 7.51 | A |
| ATOM | 1508 | O | THR | A | 211 | 39.492 | 51.420 | −5.554 | 1.00 | 7.20 | A |
| ATOM | 1509 | N | MSE | A | 212 | 37.678 | 52.017 | −4.365 | 1.00 | 8.07 | A |
| ATOM | 1510 | CA | MSE | A | 212 | 38.349 | 51.801 | −3.087 | 1.00 | 8.67 | A |
| ATOM | 1511 | CB | MSE | A | 212 | 37.480 | 52.311 | −1.933 | 1.00 | 11.85 | A |
| ATOM | 1512 | CG | MSE | A | 212 | 37.190 | 53.803 | −2.011 | 1.00 | 16.12 | A |
| ATOM | 1513 | SE | MSE | A | 212 | 38.764 | 54.895 | −2.346 | 1.00 | 25.32 | A |
| ATOM | 1514 | CE | MSE | A | 212 | 39.602 | 54.743 | −0.621 | 1.00 | 21.14 | A |
| ATOM | 1515 | C | MSE | A | 212 | 38.735 | 50.333 | −2.883 | 1.00 | 8.11 | A |
| ATOM | 1516 | O | MSE | A | 212 | 39.910 | 50.025 | −2.667 | 1.00 | 7.43 | A |
| ATOM | 1517 | N | PRO | A | 213 | 37.762 | 49.403 | −2.937 | 1.00 | 7.29 | A |
| ATOM | 1518 | CD | PRO | A | 213 | 36.293 | 49.497 | −2.956 | 1.00 | 7.20 | A |
| ATOM | 1519 | CA | PRO | A | 213 | 38.192 | 48.014 | −2.751 | 1.00 | 7.53 | A |
| ATOM | 1520 | CB | PRO | A | 213 | 36.876 | 47.222 | −2.776 | 1.00 | 8.34 | A |
| ATOM | 1521 | CG | PRO | A | 213 | 35.903 | 48.141 | −3.484 | 1.00 | 7.20 | A |
| ATOM | 1522 | C | PRO | A | 213 | 39.173 | 47.581 | −3.843 | 1.00 | 7.60 | A |
| ATOM | 1523 | O | PRO | A | 213 | 40.033 | 46.734 | −3.609 | 1.00 | 7.87 | A |
| ATOM | 1524 | N | TYR | A | 214 | 39.053 | 48.166 | −5.034 | 1.00 | 7.06 | A |
| ATOM | 1525 | CA | TYR | A | 214 | 39.960 | 47.822 | −6.131 | 1.00 | 8.43 | A |
| ATOM | 1526 | CB | TYR | A | 214 | 39.659 | 48.668 | −7.367 | 1.00 | 8.58 | A |
| ATOM | 1527 | CG | TYR | A | 214 | 40.588 | 48.408 | −8.538 | 1.00 | 9.57 | A |
| ATOM | 1528 | CD1 | TYR | A | 214 | 40.374 | 47.332 | −9.398 | 1.00 | 11.97 | A |
| ATOM | 1529 | CE1 | TYR | A | 214 | 41.196 | 47.123 | −10.507 | 1.00 | 12.57 | A |
| ATOM | 1530 | CD2 | TYR | A | 214 | 41.656 | 49.264 | −8.808 | 1.00 | 8.93 | A |
| ATOM | 1531 | CE2 | TYR | A | 214 | 42.484 | 49.063 | −9.908 | 1.00 | 10.94 | A |
| ATOM | 1532 | CZ | TYR | A | 214 | 42.246 | 47.996 | −10.757 | 1.00 | 11.50 | A |
| ATOM | 1533 | OH | TYR | A | 214 | 43.038 | 47.822 | −11.874 | 1.00 | 13.46 | A |
| ATOM | 1534 | C | TYR | A | 214 | 41.402 | 48.078 | −5.705 | 1.00 | 8.88 | A |
| ATOM | 1535 | O | TYR | A | 214 | 42.256 | 47.190 | −5.783 | 1.00 | 9.14 | A |
| ATOM | 1536 | N | ILE | A | 215 | 41.665 | 49.305 | −5.262 | 1.00 | 7.76 | A |
| ATOM | 1537 | CA | ILE | A | 215 | 42.996 | 49.702 | −4.825 | 1.00 | 7.71 | A |
| ATOM | 1538 | CB | ILE | A | 215 | 43.060 | 51.229 | −4.594 | 1.00 | 7.35 | A |
| ATOM | 1539 | CG2 | ILE | A | 215 | 44.404 | 51.616 | −3.975 | 1.00 | 7.15 | A |
| ATOM | 1540 | CG1 | ILE | A | 215 | 42.850 | 51.961 | −5.920 | 1.00 | 8.07 | A |
| ATOM | 1541 | CD1 | ILE | A | 215 | 42.746 | 53.466 | −5.778 | 1.00 | 10.41 | A |
| ATOM | 1542 | C | ILE | A | 215 | 43.410 | 48.990 | −3.536 | 1.00 | 7.79 | A |
| ATOM | 1543 | O | ILE | A | 215 | 44.530 | 48.491 | −3.424 | 1.00 | 7.37 | A |
| ATOM | 1544 | N | LEU | A | 216 | 42.500 | 48.931 | −2.571 | 1.00 | 7.76 | A |
| ATOM | 1545 | CA | LEU | A | 216 | 42.799 | 48.298 | −1.290 | 1.00 | 7.87 | A |
| ATOM | 1546 | CB | LEU | A | 216 | 41.606 | 48.452 | −0.338 | 1.00 | 7.69 | A |
| ATOM | 1547 | CG | LEU | A | 216 | 41.251 | 49.889 | 0.065 | 1.00 | 9.45 | A |
| ATOM | 1548 | CD1 | LEU | A | 216 | 39.960 | 49.897 | 0.863 | 1.00 | 10.13 | A |
| ATOM | 1549 | CD2 | LEU | A | 216 | 42.393 | 50.495 | 0.876 | 1.00 | 10.14 | A |
| ATOM | 1550 | C | LEU | A | 216 | 43.170 | 46.823 | −1.419 | 1.00 | 8.19 | A |
| ATOM | 1551 | O | LEU | A | 216 | 44.148 | 46.371 | −0.823 | 1.00 | 7.52 | A |
| ATOM | 1552 | N | GLN | A | 217 | 42.392 | 46.081 | −2.201 | 1.00 | 8.30 | A |
| ATOM | 1553 | CA | GLN | A | 217 | 42.639 | 44.654 | −2.381 | 1.00 | 8.72 | A |
| ATOM | 1554 | CB | GLN | A | 217 | 41.504 | 44.043 | −3.210 | 1.00 | 10.69 | A |
| ATOM | 1555 | CG | GLN | A | 217 | 41.475 | 42.521 | −3.264 | 1.00 | 11.14 | A |
| ATOM | 1556 | CD | GLN | A | 217 | 42.430 | 41.960 | −4.290 | 1.00 | 13.44 | A |
| ATOM | 1557 | OE1 | GLN | A | 217 | 42.639 | 42.561 | −5.342 | 1.00 | 14.29 | A |
| ATOM | 1558 | NE2 | GLN | A | 217 | 43.002 | 40.796 | −4.001 | 1.00 | 14.07 | A |
| ATOM | 1559 | C | GLN | A | 217 | 44.002 | 44.415 | −3.035 | 1.00 | 9.26 | A |
| ATOM | 1560 | O | GLN | A | 217 | 44.649 | 43.394 | −2.792 | 1.00 | 10.31 | A |
| ATOM | 1561 | N | LYS | A | 218 | 44.442 | 45.367 | −3.854 | 1.00 | 8.72 | A |
| ATOM | 1562 | CA | LYS | A | 218 | 45.738 | 45.277 | −4.522 | 1.00 | 8.85 | A |
| ATOM | 1563 | CB | LYS | A | 218 | 45.673 | 45.974 | −5.884 | 1.00 | 9.16 | A |
| ATOM | 1564 | CG | LYS | A | 218 | 44.893 | 45.182 | −6.925 | 1.00 | 9.85 | A |
| ATOM | 1565 | CD | LYS | A | 218 | 44.682 | 45.996 | −8.195 | 1.00 | 8.97 | A |
| ATOM | 1566 | CE | LYS | A | 218 | 44.293 | 45.113 | −9.372 | 1.00 | 11.30 | A |
| ATOM | 1567 | NZ | LYS | A | 218 | 43.099 | 44.252 | −9.123 | 1.00 | 12.85 | A |
| ATOM | 1568 | C | LYS | A | 218 | 46.837 | 45.899 | −3.657 | 1.00 | 9.01 | A |
| ATOM | 1569 | O | LYS | A | 218 | 47.991 | 46.024 | −4.079 | 1.00 | 8.66 | A |
| ATOM | 1570 | N | SER | A | 219 | 46.469 | 46.286 | −2.441 | 1.00 | 8.09 | A |
| ATOM | 1571 | CA | SER | A | 219 | 47.414 | 46.886 | −1.513 | 1.00 | 8.36 | A |
| ATOM | 1572 | CB | SER | A | 219 | 47.055 | 48.354 | −1.260 | 1.00 | 7.23 | A |
| ATOM | 1573 | OG | SER | A | 219 | 47.135 | 49.106 | −2.464 | 1.00 | 8.36 | A |
| ATOM | 1574 | C | SER | A | 219 | 47.467 | 46.119 | −0.192 | 1.00 | 7.81 | A |
| ATOM | 1575 | O | SER | A | 219 | 47.783 | 46.688 | 0.852 | 1.00 | 8.25 | A |
| ATOM | 1576 | N | GLY | A | 220 | 47.144 | 44.828 | −0.256 | 1.00 | 8.61 | A |
| ATOM | 1577 | CA | GLY | A | 220 | 47.196 | 43.969 | 0.918 | 1.00 | 8.94 | A |
| ATOM | 1578 | C | GLY | A | 220 | 45.996 | 43.912 | 1.847 | 1.00 | 9.36 | A |
| ATOM | 1579 | O | GLY | A | 220 | 45.996 | 43.126 | 2.794 | 1.00 | 9.85 | A |
| ATOM | 1580 | N | PHE | A | 221 | 44.971 | 44.717 | 1.593 | 1.00 | 9.14 | A |
| ATOM | 1581 | CA | PHE | A | 221 | 43.805 | 44.721 | 2.471 | 1.00 | 8.46 | A |
| ATOM | 1582 | CB | PHE | A | 221 | 42.925 | 45.940 | 2.198 | 1.00 | 7.99 | A |
| ATOM | 1583 | CG | PHE | A | 221 | 43.475 | 47.212 | 2.752 | 1.00 | 7.21 | A |
| ATOM | 1584 | CD1 | PHE | A | 221 | 44.593 | 47.813 | 2.176 | 1.00 | 7.00 | A |
| ATOM | 1585 | CD2 | PHE | A | 221 | 42.881 | 47.812 | 3.858 | 1.00 | 7.00 | A |
| ATOM | 1586 | CE1 | PHE | A | 221 | 45.112 | 48.993 | 2.695 | 1.00 | 6.41 | A |
| ATOM | 1587 | CE2 | PHE | A | 221 | 43.389 | 48.990 | 4.387 | 1.00 | 7.19 | A |
| ATOM | 1588 | CZ | PHE | A | 221 | 44.511 | 49.587 | 3.804 | 1.00 | 7.27 | A |
| ATOM | 1589 | C | PHE | A | 221 | 42.945 | 43.475 | 2.388 | 1.00 | 8.81 | A |
| ATOM | 1590 | O | PHE | A | 221 | 42.883 | 42.812 | 1.354 | 1.00 | 9.31 | A |
| ATOM | 1591 | N | LYS | A | 222 | 42.269 | 43.178 | 3.493 | 1.00 | 9.34 | A |
| ATOM | 1592 | CA | LYS | A | 222 | 41.393 | 42.024 | 3.575 | 1.00 | 10.17 | A |
| ATOM | 1593 | CB | LYS | A | 222 | 41.963 | 41.009 | 4.568 | 1.00 | 13.29 | A |
| ATOM | 1594 | CG | LYS | A | 222 | 43.259 | 40.373 | 4.095 | 1.00 | 17.15 | A |
| ATOM | 1595 | CD | LYS | A | 222 | 43.759 | 39.319 | 5.065 | 1.00 | 21.70 | A |
| ATOM | 1596 | CE | LYS | A | 222 | 44.853 | 38.475 | 4.430 | 1.00 | 23.71 | A |
| ATOM | 1597 | NZ | LYS | A | 222 | 45.964 | 39.310 | 3.898 | 1.00 | 25.58 | A |
| ATOM | 1598 | C | LYS | A | 222 | 39.976 | 42.409 | 3.988 | 1.00 | 9.40 | A |
| ATOM | 1599 | O | LYS | A | 222 | 39.045 | 41.623 | 3.817 | 1.00 | 9.41 | A |
| ATOM | 1600 | N | ASN | A | 223 | 39.810 | 43.618 | 4.520 | 1.00 | 7.78 | A |
| ATOM | 1601 | CA | ASN | A | 223 | 38.493 | 44.072 | 4.963 | 1.00 | 8.74 | A |
| ATOM | 1602 | CB | ASN | A | 223 | 38.245 | 43.690 | 6.428 | 1.00 | 8.42 | A |
| ATOM | 1603 | CG | ASN | A | 223 | 38.351 | 42.205 | 6.680 | 1.00 | 9.69 | A |
| ATOM | 1604 | OD1 | ASN | A | 223 | 39.393 | 41.707 | 7.120 | 1.00 | 12.33 | A |
| ATOM | 1605 | ND2 | ASN | A | 223 | 37.277 | 41.485 | 6.397 | 1.00 | 7.18 | A |
| ATOM | 1606 | C | ASN | A | 223 | 38.326 | 45.577 | 4.865 | 1.00 | 8.67 | A |
| ATOM | 1607 | O | ASN | A | 223 | 39.296 | 46.321 | 4.958 | 1.00 | 8.40 | A |
| ATOM | 1608 | N | MET | A | 224 | 37.084 | 46.017 | 4.696 | 1.00 | 8.46 | A |
| ATOM | 1609 | CA | MET | A | 224 | 36.789 | 47.440 | 4.639 | 1.00 | 8.92 | A |
| ATOM | 1610 | CB | MET | A | 224 | 36.907 | 47.985 | 3.205 | 1.00 | 9.87 | A |
| ATOM | 1611 | CG | MET | A | 224 | 35.838 | 47.523 | 2.223 | 1.00 | 10.12 | A |
| ATOM | 1612 | SD | MET | A | 224 | 36.006 | 48.350 | 0.600 | 1.00 | 7.76 | A |
| ATOM | 1613 | CE | MET | A | 224 | 35.537 | 49.942 | 1.024 | 1.00 | 11.95 | A |
| ATOM | 1614 | C | MET | A | 224 | 35.402 | 47.725 | 5.204 | 1.00 | 9.03 | A |
| ATOM | 1615 | O | MET | A | 224 | 34.516 | 46.857 | 5.208 | 1.00 | 9.02 | A |
| ATOM | 1616 | N | LEU | A | 225 | 35.232 | 48.942 | 5.704 | 1.00 | 8.45 | A |
| ATOM | 1617 | CA | LEU | A | 225 | 33.968 | 49.371 | 6.274 | 1.00 | 7.61 | A |
| ATOM | 1618 | CB | LEU | A | 225 | 34.106 | 49.510 | 7.795 | 1.00 | 7.96 | A |
| ATOM | 1619 | CG | LEU | A | 225 | 32.908 | 50.089 | 8.556 | 1.00 | 8.82 | A |
| ATOM | 1620 | CD1 | LEU | A | 225 | 32.890 | 49.535 | 9.972 | 1.00 | 9.70 | A |
| ATOM | 1621 | CD2 | LEU | A | 225 | 32.974 | 51.612 | 8.562 | 1.00 | 8.60 | A |
| ATOM | 1622 | C | LEU | A | 225 | 33.548 | 50.702 | 5.662 | 1.00 | 8.09 | A |
| ATOM | 1623 | O | LEU | A | 225 | 34.374 | 51.600 | 5.483 | 1.00 | 7.68 | A |
| ATOM | 1624 | N | ILE | A | 226 | 32.262 | 50.814 | 5.340 | 1.00 | 7.66 | A |
| ATOM | 1625 | CA | ILE | A | 226 | 31.702 | 52.033 | 4.768 | 1.00 | 8.84 | A |
| ATOM | 1626 | CB | ILE | A | 226 | 31.376 | 51.846 | 3.266 | 1.00 | 8.66 | A |
| ATOM | 1627 | CG2 | ILE | A | 226 | 32.658 | 51.514 | 2.510 | 1.00 | 9.89 | A |
| ATOM | 1628 | CG1 | ILE | A | 226 | 30.349 | 50.730 | 3.069 | 1.00 | 8.55 | A |
| ATOM | 1629 | CD1 | ILE | A | 226 | 29.922 | 50.548 | 1.617 | 1.00 | 10.07 | A |
| ATOM | 1630 | C | ILE | A | 226 | 30.453 | 52.428 | 5.557 | 1.00 | 8.74 | A |
| ATOM | 1631 | O | ILE | A | 226 | 29.889 | 51.610 | 6.291 | 1.00 | 8.14 | A |
| ATOM | 1632 | N | GLN | A | 227 | 30.021 | 53.676 | 5.412 | 1.00 | 9.54 | A |
| ATOM | 1633 | CA | GLN | A | 227 | 28.873 | 54.172 | 6.168 | 1.00 | 9.75 | A |
| ATOM | 1634 | CB | GLN | A | 227 | 29.391 | 54.976 | 7.372 | 1.00 | 9.88 | A |
| ATOM | 1635 | CG | GLN | A | 227 | 28.464 | 56.067 | 7.921 | 1.00 | 11.96 | A |
| ATOM | 1636 | CD | GLN | A | 227 | 27.124 | 55.547 | 8.407 | 1.00 | 12.75 | A |
| ATOM | 1637 | OE1 | GLN | A | 227 | 27.004 | 54.398 | 8.836 | 1.00 | 14.70 | A |
| ATOM | 1638 | NE2 | GLN | A | 227 | 26.108 | 56.404 | 8.362 | 1.00 | 13.56 | A |
| ATOM | 1639 | C | GLN | A | 227 | 27.846 | 55.006 | 5.405 | 1.00 | 10.51 | A |
| ATOM | 1640 | O | GLN | A | 227 | 26.645 | 54.738 | 5.481 | 1.00 | 10.20 | A |
| ATOM | 1641 | N | ARG | A | 228 | 28.299 | 56.021 | 4.679 | 1.00 | 10.04 | A |
| ATOM | 1642 | CA | ARG | A | 228 | 27.355 | 56.876 | 3.982 | 1.00 | 9.98 | A |
| ATOM | 1643 | CB | ARG | A | 228 | 27.947 | 58.273 | 3.781 | 1.00 | 9.92 | A |
| ATOM | 1644 | CG | ARG | A | 228 | 28.069 | 59.090 | 5.065 | 1.00 | 10.20 | A |
| ATOM | 1645 | CD | ARG | A | 228 | 28.533 | 60.505 | 4.744 | 1.00 | 10.13 | A |
| ATOM | 1646 | NE | ARG | A | 228 | 28.804 | 61.332 | 5.918 | 1.00 | 10.21 | A |
| ATOM | 1647 | CZ | ARG | A | 228 | 27.894 | 62.037 | 6.585 | 1.00 | 11.44 | A |
| ATOM | 1648 | NH1 | ARG | A | 228 | 26.622 | 62.022 | 6.208 | 1.00 | 12.48 | A |
| ATOM | 1649 | NH2 | ARG | A | 228 | 28.268 | 62.786 | 7.616 | 1.00 | 12.71 | A |
| ATOM | 1650 | C | ARG | A | 228 | 26.817 | 56.352 | 2.666 | 1.00 | 10.13 | A |
| ATOM | 1651 | O | ARG | A | 228 | 27.382 | 56.593 | 1.597 | 1.00 | 11.44 | A |
| ATOM | 1652 | N | THR | A | 229 | 25.714 | 55.621 | 2.770 | 1.00 | 9.97 | A |
| ATOM | 1653 | CA | THR | A | 229 | 25.025 | 55.072 | 1.617 | 1.00 | 9.89 | A |
| ATOM | 1654 | CB | THR | A | 229 | 25.125 | 53.531 | 1.560 | 1.00 | 10.00 | A |
| ATOM | 1655 | OG1 | THR | A | 229 | 24.514 | 52.964 | 2.724 | 1.00 | 10.96 | A |
| ATOM | 1656 | CG2 | THR | A | 229 | 26.583 | 53.096 | 1.493 | 1.00 | 10.02 | A |
| ATOM | 1657 | C | THR | A | 229 | 23.569 | 55.490 | 1.778 | 1.00 | 9.89 | A |
| ATOM | 1658 | O | THR | A | 229 | 23.106 | 55.742 | 2.894 | 1.00 | 11.00 | A |
| ATOM | 1659 | N | HIS | A | 230 | 22.856 | 55.573 | 0.661 | 1.00 | 9.52 | A |
| ATOM | 1660 | CA | HIS | A | 230 | 21.450 | 55.979 | 0.642 | 1.00 | 9.76 | A |
| ATOM | 1661 | CB | HIS | A | 230 | 20.883 | 55.708 | −0.754 | 1.00 | 10.27 | A |
| ATOM | 1662 | CG | HIS | A | 230 | 19.595 | 56.415 | −1.043 | 1.00 | 10.51 | A |
| ATOM | 1663 | CD2 | HIS | A | 230 | 19.286 | 57.353 | −1.969 | 1.00 | 11.88 | A |
| ATOM | 1664 | ND1 | HIS | A | 230 | 18.427 | 56.153 | −0.359 | 1.00 | 11.10 | A |
| ATOM | 1665 | CE1 | HIS | A | 230 | 17.454 | 56.899 | −0.852 | 1.00 | 11.36 | A |
| ATOM | 1666 | NE2 | HIS | A | 230 | 17.949 | 57.636 | −1.830 | 1.00 | 11.11 | A |
| ATOM | 1667 | C | HIS | A | 230 | 20.637 | 55.234 | 1.704 | 1.00 | 9.83 | A |
| ATOM | 1668 | O | HIS | A | 230 | 20.735 | 54.018 | 1.826 | 1.00 | 10.51 | A |
| ATOM | 1669 | N | TYR | A | 231 | 19.827 | 55.963 | 2.466 | 1.00 | 10.15 | A |
| ATOM | 1670 | CA | TYR | A | 231 | 19.030 | 55.327 | 3.511 | 1.00 | 10.13 | A |
| ATOM | 1671 | CB | TYR | A | 231 | 18.178 | 56.372 | 4.246 | 1.00 | 10.68 | A |
| ATOM | 1672 | CG | TYR | A | 231 | 17.197 | 57.136 | 3.381 | 1.00 | 11.51 | A |
| ATOM | 1673 | CD1 | TYR | A | 231 | 15.910 | 56.648 | 3.151 | 1.00 | 10.48 | A |
| ATOM | 1674 | CE1 | TYR | A | 231 | 14.997 | 57.361 | 2.372 | 1.00 | 11.82 | A |
| ATOM | 1675 | CD2 | TYR | A | 231 | 17.552 | 58.359 | 2.805 | 1.00 | 11.23 | A |
| ATOM | 1676 | CE2 | TYR | A | 231 | 16.649 | 59.079 | 2.022 | 1.00 | 11.82 | A |
| ATOM | 1677 | CZ | TYR | A | 231 | 15.373 | 58.573 | 1.813 | 1.00 | 11.99 | A |
| ATOM | 1678 | OH | TYR | A | 231 | 14.470 | 59.278 | 1.054 | 1.00 | 12.91 | A |
| ATOM | 1679 | C | TYR | A | 231 | 18.155 | 54.188 | 2.984 | 1.00 | 11.22 | A |
| ATOM | 1680 | O | TYR | A | 231 | 17.897 | 53.220 | 3.699 | 1.00 | 12.09 | A |
| ATOM | 1681 | N | SER | A | 232 | 17.710 | 54.288 | 1.736 | 1.00 | 10.94 | A |
| ATOM | 1682 | CA | SER | A | 232 | 16.876 | 53.238 | 1.155 | 1.00 | 11.93 | A |
| ATOM | 1683 | CB | SER | A | 232 | 16.257 | 53.708 | −0.163 | 1.00 | 12.92 | A |
| ATOM | 1684 | OG | SER | A | 232 | 15.307 | 54.731 | 0.063 | 1.00 | 15.26 | A |
| ATOM | 1685 | C | SER | A | 232 | 17.692 | 51.976 | 0.915 | 1.00 | 11.85 | A |
| ATOM | 1686 | O | SER | A | 232 | 17.176 | 50.861 | 1.025 | 1.00 | 11.64 | A |
| ATOM | 1687 | N | VAL | A | 233 | 18.967 | 52.158 | 0.580 | 1.00 | 11.60 | A |
| ATOM | 1688 | CA | VAL | A | 233 | 19.868 | 51.038 | 0.336 | 1.00 | 11.31 | A |
| ATOM | 1689 | CB | VAL | A | 233 | 21.197 | 51.527 | −0.295 | 1.00 | 10.85 | A |
| ATOM | 1690 | CG1 | VAL | A | 233 | 22.222 | 50.406 | −0.300 | 1.00 | 10.34 | A |
| ATOM | 1691 | CG2 | VAL | A | 233 | 20.945 | 51.999 | −1.724 | 1.00 | 11.98 | A |
| ATOM | 1692 | C | VAL | A | 233 | 20.152 | 50.315 | 1.653 | 1.00 | 11.28 | A |
| ATOM | 1693 | O | VAL | A | 233 | 20.166 | 49.085 | 1.706 | 1.00 | 11.15 | A |
| ATOM | 1694 | N | LYS | A | 234 | 20.379 | 51.080 | 2.716 | 1.00 | 10.86 | A |
| ATOM | 1695 | CA | LYS | A | 234 | 20.632 | 50.477 | 4.020 | 1.00 | 10.69 | A |
| ATOM | 1696 | CB | LYS | A | 234 | 20.862 | 51.563 | 5.075 | 1.00 | 10.64 | A |
| ATOM | 1697 | CG | LYS | A | 234 | 22.199 | 52.286 | 4.946 | 1.00 | 11.25 | A |
| ATOM | 1698 | CD | LYS | A | 234 | 22.250 | 53.516 | 5.841 | 1.00 | 11.31 | A |
| ATOM | 1699 | CE | LYS | A | 234 | 23.624 | 54.189 | 5.810 | 1.00 | 10.54 | A |
| ATOM | 1700 | NZ | LYS | A | 234 | 24.597 | 53.574 | 6.772 | 1.00 | 10.11 | A |
| ATOM | 1701 | C | LYS | A | 234 | 19.443 | 49.609 | 4.424 | 1.00 | 11.48 | A |
| ATOM | 1702 | O | LYS | A | 234 | 19.619 | 48.484 | 4.884 | 1.00 | 11.20 | A |
| ATOM | 1703 | N | LYS | A | 235 | 18.236 | 50.135 | 4.238 | 1.00 | 11.59 | A |
| ATOM | 1704 | CA | LYS | A | 235 | 17.024 | 49.400 | 4.595 | 1.00 | 12.69 | A |
| ATOM | 1705 | CB | LYS | A | 235 | 15.789 | 50.286 | 4.401 | 1.00 | 13.05 | A |
| ATOM | 1706 | CG | LYS | A | 235 | 14.479 | 49.630 | 4.827 | 1.00 | 13.80 | A |
| ATOM | 1707 | CD | LYS | A | 235 | 13.312 | 50.593 | 4.692 | 1.00 | 14.78 | A |
| ATOM | 1708 | CE | LYS | A | 235 | 12.016 | 49.963 | 5.174 | 1.00 | 16.08 | A |
| ATOM | 1709 | NZ | LYS | A | 235 | 10.853 | 50.872 | 4.963 | 1.00 | 18.03 | A |
| ATOM | 1710 | C | LYS | A | 235 | 16.878 | 48.122 | 3.775 | 1.00 | 12.66 | A |
| ATOM | 1711 | O | LYS | A | 235 | 16.596 | 47.056 | 4.322 | 1.00 | 13.44 | A |
| ATOM | 1712 | N | GLU | A | 236 | 17.084 | 48.230 | 2.467 | 1.00 | 13.43 | A |
| ATOM | 1713 | CA | GLU | A | 236 | 16.962 | 47.085 | 1.572 | 1.00 | 13.76 | A |
| ATOM | 1714 | CB | GLU | A | 236 | 17.160 | 47.531 | 0.119 | 1.00 | 15.66 | A |
| ATOM | 1715 | CG | GLU | A | 236 | 16.930 | 46.435 | −0.919 | 1.00 | 19.35 | A |
| ATOM | 1716 | CD | GLU | A | 236 | 15.454 | 46.120 | −1.139 | 1.00 | 21.36 | A |
| ATOM | 1717 | OE1 | GLU | A | 236 | 15.157 | 45.192 | −1.921 | 1.00 | 23.68 | A |
| ATOM | 1718 | OE2 | GLU | A | 236 | 14.594 | 46.799 | −0.539 | 1.00 | 23.54 | A |
| ATOM | 1719 | C | GLU | A | 236 | 17.963 | 45.983 | 1.908 | 1.00 | 13.53 | A |
| ATOM | 1720 | O | GLU | A | 236 | 17.596 | 44.817 | 2.035 | 1.00 | 13.84 | A |
| ATOM | 1721 | N | LEU | A | 237 | 19.232 | 46.346 | 2.051 | 1.00 | 12.28 | A |
| ATOM | 1722 | CA | LEU | A | 237 | 20.251 | 45.355 | 2.364 | 1.00 | 11.83 | A |
| ATOM | 1723 | CB | LEU | A | 237 | 21.649 | 45.952 | 2.180 | 1.00 | 11.01 | A |
| ATOM | 1724 | CG | LEU | A | 237 | 21.993 | 46.436 | 0.767 | 1.00 | 12.53 | A |
| ATOM | 1725 | CD1 | LEU | A | 237 | 23.396 | 47.036 | 0.776 | 1.00 | 13.20 | A |
| ATOM | 1726 | CD2 | LEU | A | 237 | 21.905 | 45.280 | −0.227 | 1.00 | 12.87 | A |
| ATOM | 1727 | C | LEU | A | 237 | 20.094 | 44.808 | 3.781 | 1.00 | 11.90 | A |
| ATOM | 1728 | O | LEU | A | 237 | 20.376 | 43.640 | 4.032 | 1.00 | 12.05 | A |
| ATOM | 1729 | N | ALA | A | 238 | 19.641 | 45.647 | 4.706 | 1.00 | 12.02 | A |
| ATOM | 1730 | CA | ALA | A | 238 | 19.456 | 45.205 | 6.084 | 1.00 | 12.54 | A |
| ATOM | 1731 | CB | ALA | A | 238 | 19.050 | 46.380 | 6.962 | 1.00 | 12.67 | A |
| ATOM | 1732 | C | ALA | A | 238 | 18.389 | 44.115 | 6.146 | 1.00 | 13.90 | A |
| ATOM | 1733 | O | ALA | A | 238 | 18.547 | 43.120 | 6.853 | 1.00 | 13.71 | A |
| ATOM | 1734 | N | GLN | A | 239 | 17.309 | 44.304 | 5.396 | 1.00 | 14.65 | A |
| ATOM | 1735 | CA | GLN | A | 239 | 16.216 | 43.335 | 5.391 | 1.00 | 16.12 | A |
| ATOM | 1736 | CB | GLN | A | 239 | 15.050 | 43.858 | 4.544 | 1.00 | 17.16 | A |
| ATOM | 1737 | CG | GLN | A | 239 | 14.476 | 45.180 | 5.040 | 1.00 | 20.68 | A |
| ATOM | 1738 | CD | GLN | A | 239 | 13.320 | 45.684 | 4.193 | 1.00 | 21.84 | A |
| ATOM | 1739 | OE1 | GLN | A | 239 | 13.385 | 45.678 | 2.962 | 1.00 | 24.34 | A |
| ATOM | 1740 | NE2 | GLN | A | 239 | 12.258 | 46.139 | 4.852 | 1.00 | 23.09 | A |
| ATOM | 1741 | C | GLN | A | 239 | 16.649 | 41.959 | 4.888 | 1.00 | 16.39 | A |
| ATOM | 1742 | O | GLN | A | 239 | 16.067 | 40.944 | 5.267 | 1.00 | 16.74 | A |
| ATOM | 1743 | N | GLN | A | 240 | 17.673 | 41.923 | 4.042 | 1.00 | 15.26 | A |
| ATOM | 1744 | CA | GLN | A | 240 | 18.164 | 40.661 | 3.493 | 1.00 | 15.33 | A |
| ATOM | 1745 | CB | GLN | A | 240 | 18.389 | 40.803 | 1.986 | 1.00 | 17.66 | A |
| ATOM | 1746 | CG | GLN | A | 240 | 17.204 | 41.396 | 1.241 | 1.00 | 20.48 | A |
| ATOM | 1747 | CD | GLN | A | 240 | 15.975 | 40.514 | 1.292 | 1.00 | 22.28 | A |
| ATOM | 1748 | OE1 | GLN | A | 240 | 14.857 | 40.979 | 1.065 | 1.00 | 25.19 | A |
| ATOM | 1749 | NE2 | GLN | A | 240 | 16.173 | 39.232 | 1.579 | 1.00 | 23.21 | A |
| ATOM | 1750 | C | GLN | A | 240 | 19.471 | 40.250 | 4.155 | 1.00 | 14.44 | A |
| ATOM | 1751 | O | GLN | A | 240 | 20.100 | 39.274 | 3.748 | 1.00 | 13.55 | A |
| ATOM | 1752 | N | ARG | A | 241 | 19.863 | 40.991 | 5.187 | 1.00 | 13.13 | A |
| ATOM | 1753 | CA | ARG | A | 241 | 21.115 | 40.740 | 5.889 | 1.00 | 12.19 | A |
| ATOM | 1754 | CB | ARG | A | 241 | 21.053 | 39.430 | 6.684 | 1.00 | 13.02 | A |
| ATOM | 1755 | CG | ARG | A | 241 | 20.025 | 39.457 | 7.810 | 1.00 | 15.06 | A |
| ATOM | 1756 | CD | ARG | A | 241 | 20.249 | 38.323 | 8.798 | 1.00 | 16.85 | A |
| ATOM | 1757 | NE | ARG | A | 241 | 20.265 | 37.021 | 8.141 | 1.00 | 19.77 | A |
| ATOM | 1758 | CZ | ARG | A | 241 | 20.649 | 35.894 | 8.732 | 1.00 | 20.67 | A |
| ATOM | 1759 | NH1 | ARG | A | 241 | 21.049 | 35.910 | 9.997 | 1.00 | 21.43 | A |
| ATOM | 1760 | NH2 | ARG | A | 241 | 20.641 | 34.754 | 8.056 | 1.00 | 21.54 | A |
| ATOM | 1761 | C | ARG | A | 241 | 22.254 | 40.694 | 4.876 | 1.00 | 11.37 | A |
| ATOM | 1762 | O | ARG | A | 241 | 23.069 | 39.774 | 4.863 | 1.00 | 10.26 | A |
| ATOM | 1763 | N | GLN | A | 242 | 22.290 | 41.706 | 4.016 | 1.00 | 11.13 | A |
| ATOM | 1764 | CA | GLN | A | 242 | 23.323 | 41.811 | 2.995 | 1.00 | 10.98 | A |
| ATOM | 1765 | CB | GLN | A | 242 | 22.682 | 41.863 | 1.602 | 1.00 | 11.23 | A |
| ATOM | 1766 | CG | GLN | A | 242 | 21.894 | 40.607 | 1.237 | 1.00 | 11.67 | A |
| ATOM | 1767 | CD | GLN | A | 242 | 21.187 | 40.729 | −0.098 | 1.00 | 11.70 | A |
| ATOM | 1768 | OE1 | GLN | A | 242 | 20.617 | 41.770 | −0.412 | 1.00 | 13.37 | A |
| ATOM | 1769 | NE2 | GLN | A | 242 | 21.208 | 39.656 | −0.884 | 1.00 | 12.95 | A |
| ATOM | 1770 | C | GLN | A | 242 | 24.178 | 43.055 | 3.233 | 1.00 | 10.67 | A |
| ATOM | 1771 | O | GLN | A | 242 | 24.695 | 43.651 | 2.289 | 1.00 | 10.60 | A |
| ATOM | 1772 | N | LEU | A | 243 | 24.322 | 43.440 | 4.500 | 1.00 | 10.44 | A |
| ATOM | 1773 | CA | LEU | A | 243 | 25.128 | 44.606 | 4.852 | 1.00 | 9.93 | A |
| ATOM | 1774 | CB | LEU | A | 243 | 24.704 | 45.154 | 6.216 | 1.00 | 10.62 | A |
| ATOM | 1775 | CG | LEU | A | 243 | 23.328 | 45.826 | 6.244 | 1.00 | 11.00 | A |
| ATOM | 1776 | CD1 | LEU | A | 243 | 22.896 | 46.028 | 7.688 | 1.00 | 11.97 | A |
| ATOM | 1777 | CD2 | LEU | A | 243 | 23.370 | 47.158 | 5.490 | 1.00 | 11.86 | A |
| ATOM | 1778 | C | LEU | A | 243 | 26.613 | 44.260 | 4.853 | 1.00 | 10.25 | A |
| ATOM | 1779 | O | LEU | A | 243 | 27.465 | 45.144 | 4.827 | 1.00 | 10.36 | A |
| ATOM | 1780 | N | GLU | A | 244 | 26.922 | 42.969 | 4.911 | 1.00 | 9.67 | A |
| ATOM | 1781 | CA | GLU | A | 244 | 28.306 | 42.519 | 4.841 | 1.00 | 8.98 | A |
| ATOM | 1782 | CB | GLU | A | 244 | 28.693 | 41.695 | 6.073 | 1.00 | 9.51 | A |
| ATOM | 1783 | CG | GLU | A | 244 | 28.875 | 42.595 | 7.291 | 1.00 | 10.20 | A |
| ATOM | 1784 | CD | GLU | A | 244 | 29.326 | 41.864 | 8.538 | 1.00 | 10.87 | A |
| ATOM | 1785 | OE1 | GLU | A | 244 | 28.790 | 40.773 | 8.818 | 1.00 | 11.28 | A |
| ATOM | 1786 | OE2 | GLU | A | 244 | 30.207 | 42.402 | 9.249 | 1.00 | 10.96 | A |
| ATOM | 1787 | C | GLU | A | 244 | 28.339 | 41.703 | 3.569 | 1.00 | 9.24 | A |
| ATOM | 1788 | O | GLU | A | 244 | 27.536 | 40.788 | 3.386 | 1.00 | 10.29 | A |
| ATOM | 1789 | N | PHE | A | 245 | 29.260 | 42.053 | 2.680 | 1.00 | 8.67 | A |
| ATOM | 1790 | CA | PHE | A | 245 | 29.337 | 41.396 | 1.389 | 1.00 | 8.89 | A |
| ATOM | 1791 | CB | PHE | A | 245 | 28.379 | 42.114 | 0.431 | 1.00 | 8.55 | A |
| ATOM | 1792 | CG | PHE | A | 245 | 28.521 | 43.615 | 0.444 | 1.00 | 8.85 | A |
| ATOM | 1793 | CD1 | PHE | A | 245 | 29.493 | 44.250 | −0.327 | 1.00 | 8.41 | A |
| ATOM | 1794 | CD2 | PHE | A | 245 | 27.689 | 44.392 | 1.245 | 1.00 | 8.58 | A |
| ATOM | 1795 | CE1 | PHE | A | 245 | 29.631 | 45.646 | −0.300 | 1.00 | 8.68 | A |
| ATOM | 1796 | CE2 | PHE | A | 245 | 27.817 | 45.782 | 1.283 | 1.00 | 8.53 | A |
| ATOM | 1797 | CZ | PHE | A | 245 | 28.790 | 46.409 | 0.509 | 1.00 | 8.54 | A |
| ATOM | 1798 | C | PHE | A | 245 | 30.730 | 41.382 | 0.793 | 1.00 | 8.60 | A |
| ATOM | 1799 | O | PHE | A | 245 | 31.619 | 42.115 | 1.235 | 1.00 | 8.82 | A |
| ATOM | 1800 | N | LEU | A | 246 | 30.917 | 40.532 | −0.212 | 1.00 | 8.87 | A |
| ATOM | 1801 | CA | LEU | A | 246 | 32.187 | 40.446 | −0.913 | 1.00 | 8.06 | A |
| ATOM | 1802 | CB | LEU | A | 246 | 32.466 | 39.001 | −1.333 | 1.00 | 9.53 | A |
| ATOM | 1803 | CG | LEU | A | 246 | 32.800 | 38.076 | −0.154 | 1.00 | 11.53 | A |
| ATOM | 1804 | CD1 | LEU | A | 246 | 32.772 | 36.626 | −0.602 | 1.00 | 13.79 | A |
| ATOM | 1805 | CD2 | LEU | A | 246 | 34.166 | 38.447 | 0.402 | 1.00 | 13.15 | A |
| ATOM | 1806 | C | LEU | A | 246 | 32.033 | 41.364 | −2.120 | 1.00 | 8.24 | A |
| ATOM | 1807 | O | LEU | A | 246 | 31.392 | 41.019 | −3.115 | 1.00 | 8.61 | A |
| ATOM | 1808 | N | TRP | A | 247 | 32.611 | 42.552 | −2.002 | 1.00 | 7.63 | A |
| ATOM | 1809 | CA | TRP | A | 247 | 32.522 | 43.574 | −3.037 | 1.00 | 7.37 | A |
| ATOM | 1810 | CB | TRP | A | 247 | 32.718 | 44.947 | −2.388 | 1.00 | 7.55 | A |
| ATOM | 1811 | CG | TRP | A | 247 | 32.238 | 46.118 | −3.199 | 1.00 | 7.27 | A |
| ATOM | 1812 | CD2 | TRP | A | 247 | 32.240 | 47.492 | −2.791 | 1.00 | 7.02 | A |
| ATOM | 1813 | CE2 | TRP | A | 247 | 31.685 | 48.247 | −3.850 | 1.00 | 7.18 | A |
| ATOM | 1814 | CE3 | TRP | A | 247 | 32.659 | 48.160 | −1.631 | 1.00 | 7.47 | A |
| ATOM | 1815 | CD1 | TRP | A | 247 | 31.699 | 46.093 | −4.455 | 1.00 | 7.86 | A |
| ATOM | 1816 | NE1 | TRP | A | 247 | 31.363 | 47.371 | −4.853 | 1.00 | 7.55 | A |
| ATOM | 1817 | CZ2 | TRP | A | 247 | 31.538 | 49.638 | −3.784 | 1.00 | 8.05 | A |
| ATOM | 1818 | CZ3 | TRP | A | 247 | 32.514 | 49.542 | −1.565 | 1.00 | 6.90 | A |
| ATOM | 1819 | CH2 | TRP | A | 247 | 31.958 | 50.267 | −2.636 | 1.00 | 7.34 | A |
| ATOM | 1820 | C | TRP | A | 247 | 33.546 | 43.372 | −4.145 | 1.00 | 7.92 | A |
| ATOM | 1821 | O | TRP | A | 247 | 34.749 | 43.559 | −3.939 | 1.00 | 8.05 | A |
| ATOM | 1822 | N | ARG | A | 248 | 33.062 | 42.993 | −5.326 | 1.00 | 7.42 | A |
| ATOM | 1823 | CA | ARG | A | 248 | 33.932 | 42.786 | −6.477 | 1.00 | 7.87 | A |
| ATOM | 1824 | CB | ARG | A | 248 | 33.690 | 41.415 | −7.109 | 1.00 | 8.53 | A |
| ATOM | 1825 | CG | ARG | A | 248 | 32.327 | 41.277 | −7.775 | 1.00 | 8.64 | A |
| ATOM | 1826 | CD | ARG | A | 248 | 32.236 | 39.987 | −8.581 | 1.00 | 10.45 | A |
| ATOM | 1827 | NE | ARG | A | 248 | 32.326 | 38.807 | −7.727 | 1.00 | 11.08 | A |
| ATOM | 1828 | CZ | ARG | A | 248 | 32.317 | 37.558 | −8.185 | 1.00 | 12.28 | A |
| ATOM | 1829 | NH1 | ARG | A | 248 | 32.228 | 37.330 | −9.488 | 1.00 | 13.21 | A |
| ATOM | 1830 | NH2 | ARG | A | 248 | 32.382 | 36.537 | −7.339 | 1.00 | 12.78 | A |
| ATOM | 1831 | C | ARG | A | 248 | 33.645 | 43.856 | −7.521 | 1.00 | 7.64 | A |
| ATOM | 1832 | O | ARG | A | 248 | 32.605 | 44.515 | −7.474 | 1.00 | 7.91 | A |
| ATOM | 1833 | N | GLN | A | 249 | 34.560 | 44.010 | −8.472 | 1.00 | 7.42 | A |
| ATOM | 1834 | CA | GLN | A | 249 | 34.389 | 44.994 | −9.531 | 1.00 | 7.77 | A |
| ATOM | 1835 | CB | GLN | A | 249 | 35.713 | 45.188 | −10.272 | 1.00 | 8.10 | A |
| ATOM | 1836 | CG | GLN | A | 249 | 36.832 | 45.675 | −9.353 | 1.00 | 8.22 | A |
| ATOM | 1837 | CD | GLN | A | 249 | 36.448 | 46.940 | −8.598 | 1.00 | 8.40 | A |
| ATOM | 1838 | OE1 | GLN | A | 249 | 36.432 | 46.970 | −7.358 | 1.00 | 10.28 | A |
| ATOM | 1839 | NE2 | GLN | A | 249 | 36.136 | 47.993 | −9.341 | 1.00 | 7.14 | A |
| ATOM | 1840 | C | GLN | A | 249 | 33.280 | 44.546 | −10.480 | 1.00 | 8.70 | A |
| ATOM | 1841 | O | GLN | A | 249 | 33.042 | 43.349 | −10.654 | 1.00 | 9.20 | A |
| ATOM | 1842 | N | ILE | A | 250 | 32.600 | 45.511 | −11.093 | 1.00 | 9.12 | A |
| ATOM | 1843 | CA | ILE | A | 250 | 31.480 | 45.200 | −11.979 | 1.00 | 9.19 | A |
| ATOM | 1844 | CB | ILE | A | 250 | 30.791 | 46.503 | −12.492 | 1.00 | 9.85 | A |
| ATOM | 1845 | CG2 | ILE | A | 250 | 30.175 | 47.259 | −11.313 | 1.00 | 10.38 | A |
| ATOM | 1846 | CG1 | ILE | A | 250 | 31.797 | 47.390 | −13.227 | 1.00 | 11.20 | A |
| ATOM | 1847 | CD1 | ILE | A | 250 | 31.197 | 48.698 | −13.725 | 1.00 | 11.47 | A |
| ATOM | 1848 | C | ILE | A | 250 | 31.780 | 44.286 | −13.169 | 1.00 | 10.02 | A |
| ATOM | 1849 | O | ILE | A | 250 | 30.878 | 43.622 | −13.675 | 1.00 | 10.72 | A |
| ATOM | 1850 | N | TRP | A | 251 | 33.037 | 44.231 | −13.594 | 1.00 | 10.74 | A |
| ATOM | 1851 | CA | TRP | A | 251 | 33.428 | 43.399 | −14.736 | 1.00 | 12.59 | A |
| ATOM | 1852 | CB | TRP | A | 251 | 34.414 | 44.165 | −15.611 | 1.00 | 12.54 | A |
| ATOM | 1853 | CG | TRP | A | 251 | 35.724 | 44.337 | −14.919 | 1.00 | 14.60 | A |
| ATOM | 1854 | CD2 | TRP | A | 251 | 36.151 | 45.478 | −14.175 | 1.00 | 14.42 | A |
| ATOM | 1855 | CE2 | TRP | A | 251 | 37.400 | 45.159 | −13.599 | 1.00 | 15.03 | A |
| ATOM | 1856 | CE3 | TRP | A | 251 | 35.597 | 46.742 | −13.934 | 1.00 | 14.69 | A |
| ATOM | 1857 | CD1 | TRP | A | 251 | 36.708 | 43.397 | −14.775 | 1.00 | 15.34 | A |
| ATOM | 1858 | NE1 | TRP | A | 251 | 37.715 | 43.882 | −13.982 | 1.00 | 15.12 | A |
| ATOM | 1859 | CZ2 | TRP | A | 251 | 38.107 | 46.058 | −12.795 | 1.00 | 15.07 | A |
| ATOM | 1860 | CZ3 | TRP | A | 251 | 36.299 | 47.636 | −13.136 | 1.00 | 15.29 | A |
| ATOM | 1861 | CH2 | TRP | A | 251 | 37.541 | 47.288 | −12.577 | 1.00 | 15.76 | A |
| ATOM | 1862 | C | TRP | A | 251 | 34.096 | 42.089 | −14.328 | 1.00 | 13.99 | A |
| ATOM | 1863 | O | TRP | A | 251 | 34.429 | 41.268 | −15.182 | 1.00 | 13.91 | A |
| ATOM | 1864 | N | ASP | A | 252 | 34.301 | 41.903 | −13.030 | 1.00 | 13.53 | A |
| ATOM | 1865 | CA | ASP | A | 252 | 34.982 | 40.719 | −12.509 | 1.00 | 14.68 | A |
| ATOM | 1866 | CB | ASP | A | 252 | 35.582 | 41.062 | −11.142 | 1.00 | 13.69 | A |
| ATOM | 1867 | CG | ASP | A | 252 | 36.337 | 39.907 | −10.523 | 1.00 | 15.31 | A |
| ATOM | 1868 | OD1 | ASP | A | 252 | 36.637 | 38.929 | −11.241 | 1.00 | 16.51 | A |
| ATOM | 1869 | OD2 | ASP | A | 252 | 36.637 | 39.988 | −9.313 | 1.00 | 15.02 | A |
| ATOM | 1870 | C | ASP | A | 252 | 34.111 | 39.469 | −12.413 | 1.00 | 15.48 | A |
| ATOM | 1871 | O | ASP | A | 252 | 33.357 | 39.291 | −11.459 | 1.00 | 15.34 | A |
| ATOM | 1872 | N | ASN | A | 253 | 34.239 | 38.587 | −13.398 | 1.00 | 16.95 | A |
| ATOM | 1873 | CA | ASN | A | 253 | 33.440 | 37.371 | −13.420 | 1.00 | 18.55 | A |
| ATOM | 1874 | CB | ASN | A | 253 | 33.378 | 36.815 | −14.844 | 1.00 | 20.21 | A |
| ATOM | 1875 | CG | ASN | A | 253 | 32.069 | 36.113 | −15.132 | 1.00 | 22.48 | A |
| ATOM | 1876 | OD1 | ASN | A | 253 | 31.805 | 35.031 | −14.615 | 1.00 | 24.09 | A |
| ATOM | 1877 | ND2 | ASN | A | 253 | 31.231 | 36.740 | −15.950 | 1.00 | 23.35 | A |
| ATOM | 1878 | C | ASN | A | 253 | 33.948 | 36.293 | −12.466 | 1.00 | 18.73 | A |
| ATOM | 1879 | O | ASN | A | 253 | 33.158 | 35.532 | −11.911 | 1.00 | 19.87 | A |
| ATOM | 1880 | N | LYS | A | 254 | 35.261 | 36.235 | −12.269 | 1.00 | 19.53 | A |
| ATOM | 1881 | CA | LYS | A | 254 | 35.850 | 35.229 | −11.388 | 1.00 | 20.76 | A |
| ATOM | 1882 | CB | LYS | A | 254 | 37.322 | 35.010 | −11.751 | 1.00 | 23.00 | A |
| ATOM | 1883 | CG | LYS | A | 254 | 37.954 | 33.785 | −11.101 | 1.00 | 25.67 | A |
| ATOM | 1884 | CD | LYS | A | 254 | 39.370 | 33.561 | −11.616 | 1.00 | 27.42 | A |
| ATOM | 1885 | CE | LYS | A | 254 | 39.961 | 32.259 | −11.089 | 1.00 | 28.32 | A |
| ATOM | 1886 | NZ | LYS | A | 254 | 40.034 | 32.228 | −9.602 | 1.00 | 28.78 | A |
| ATOM | 1887 | C | LYS | A | 254 | 35.730 | 35.616 | −9.916 | 1.00 | 20.23 | A |
| ATOM | 1888 | O | LYS | A | 254 | 35.481 | 34.762 | −9.061 | 1.00 | 20.70 | A |
| ATOM | 1889 | N | GLY | A | 255 | 35.914 | 36.901 | −9.624 | 1.00 | 19.06 | A |
| ATOM | 1890 | CA | GLY | A | 255 | 35.811 | 37.372 | −8.253 | 1.00 | 17.83 | A |
| ATOM | 1891 | C | GLY | A | 255 | 37.130 | 37.609 | −7.534 | 1.00 | 17.06 | A |
| ATOM | 1892 | O | GLY | A | 255 | 37.140 | 37.810 | −6.320 | 1.00 | 16.43 | A |
| ATOM | 1893 | N | ASP | A | 256 | 38.239 | 37.608 | −8.268 | 1.00 | 16.71 | A |
| ATOM | 1894 | CA | ASP | A | 256 | 39.548 | 37.818 | −7.652 | 1.00 | 17.09 | A |
| ATOM | 1895 | CB | ASP | A | 256 | 40.670 | 37.546 | −8.658 | 1.00 | 20.30 | A |
| ATOM | 1896 | CG | ASP | A | 256 | 40.680 | 36.113 | −9.147 | 1.00 | 22.97 | A |
| ATOM | 1897 | OD1 | ASP | A | 256 | 40.535 | 35.196 | −8.309 | 1.00 | 25.34 | A |
| ATOM | 1898 | OD2 | ASP | A | 256 | 40.844 | 35.904 | −10.368 | 1.00 | 25.26 | A |
| ATOM | 1899 | C | ASP | A | 256 | 39.738 | 39.212 | −7.059 | 1.00 | 15.66 | A |
| ATOM | 1900 | O | ASP | A | 256 | 40.640 | 39.426 | −6.253 | 1.00 | 15.19 | A |
| ATOM | 1901 | N | THR | A | 257 | 38.900 | 40.163 | −7.457 | 1.00 | 13.06 | A |
| ATOM | 1902 | CA | THR | A | 257 | 39.010 | 41.523 | −6.926 | 1.00 | 12.42 | A |
| ATOM | 1903 | CB | THR | A | 257 | 38.501 | 42.579 | −7.926 | 1.00 | 12.11 | A |
| ATOM | 1904 | OG1 | THR | A | 257 | 37.112 | 42.348 | −8.194 | 1.00 | 11.97 | A |
| ATOM | 1905 | CG2 | THR | A | 257 | 39.303 | 42.528 | −9.218 | 1.00 | 12.56 | A |
| ATOM | 1906 | C | THR | A | 257 | 38.204 | 41.716 | −5.644 | 1.00 | 12.57 | A |
| ATOM | 1907 | O | THR | A | 257 | 38.301 | 42.759 | −5.000 | 1.00 | 13.02 | A |
| ATOM | 1908 | N | ALA | A | 258 | 37.420 | 40.707 | −5.277 | 1.00 | 11.47 | A |
| ATOM | 1909 | CA | ALA | A | 258 | 36.568 | 40.785 | −4.096 | 1.00 | 10.47 | A |
| ATOM | 1910 | CB | ALA | A | 258 | 35.819 | 39.475 | −3.907 | 1.00 | 11.26 | A |
| ATOM | 1911 | C | ALA | A | 258 | 37.262 | 41.162 | −2.794 | 1.00 | 10.09 | A |
| ATOM | 1912 | O | ALA | A | 258 | 38.349 | 40.675 | −2.481 | 1.00 | 11.10 | A |
| ATOM | 1913 | N | LEU | A | 259 | 36.608 | 42.038 | −2.041 | 1.00 | 9.25 | A |
| ATOM | 1914 | CA | LEU | A | 259 | 37.106 | 42.486 | −0.747 | 1.00 | 9.11 | A |
| ATOM | 1915 | CB | LEU | A | 259 | 37.709 | 43.889 | −0.846 | 1.00 | 8.75 | A |
| ATOM | 1916 | CG | LEU | A | 259 | 38.434 | 44.373 | 0.416 | 1.00 | 8.50 | A |
| ATOM | 1917 | CD1 | LEU | A | 259 | 39.634 | 43.466 | 0.702 | 1.00 | 9.84 | A |
| ATOM | 1918 | CD2 | LEU | A | 259 | 38.890 | 45.818 | 0.226 | 1.00 | 10.21 | A |
| ATOM | 1919 | C | LEU | A | 259 | 35.923 | 42.506 | 0.211 | 1.00 | 8.64 | A |
| ATOM | 1920 | O | LEU | A | 259 | 34.890 | 43.119 | −0.074 | 1.00 | 8.01 | A |
| ATOM | 1921 | N | PHE | A | 260 | 36.068 | 41.829 | 1.347 | 1.00 | 8.58 | A |
| ATOM | 1922 | CA | PHE | A | 260 | 34.997 | 41.785 | 2.333 | 1.00 | 8.58 | A |
| ATOM | 1923 | CB | PHE | A | 260 | 35.405 | 40.932 | 3.533 | 1.00 | 8.62 | A |
| ATOM | 1924 | CG | PHE | A | 260 | 34.301 | 40.738 | 4.529 | 1.00 | 9.48 | A |
| ATOM | 1925 | CD1 | PHE | A | 260 | 33.310 | 39.786 | 4.310 | 1.00 | 11.03 | A |
| ATOM | 1926 | CD2 | PHE | A | 260 | 34.233 | 41.523 | 5.670 | 1.00 | 9.44 | A |
| ATOM | 1927 | CE1 | PHE | A | 260 | 32.269 | 39.619 | 5.217 | 1.00 | 10.46 | A |
| ATOM | 1928 | CE2 | PHE | A | 260 | 33.194 | 41.366 | 6.587 | 1.00 | 10.60 | A |
| ATOM | 1929 | CZ | PHE | A | 260 | 32.210 | 40.413 | 6.358 | 1.00 | 10.54 | A |
| ATOM | 1930 | C | PHE | A | 260 | 34.669 | 43.198 | 2.800 | 1.00 | 7.96 | A |
| ATOM | 1931 | O | PHE | A | 260 | 35.548 | 43.943 | 3.240 | 1.00 | 8.25 | A |
| ATOM | 1932 | N | THR | A | 261 | 33.393 | 43.555 | 2.723 | 1.00 | 7.64 | A |
| ATOM | 1933 | CA | THR | A | 261 | 32.951 | 44.887 | 3.098 | 1.00 | 7.65 | A |
| ATOM | 1934 | CB | THR | A | 261 | 32.467 | 45.656 | 1.849 | 1.00 | 8.01 | A |
| ATOM | 1935 | OG1 | THR | A | 261 | 33.522 | 45.707 | 0.881 | 1.00 | 8.64 | A |
| ATOM | 1936 | CG2 | THR | A | 261 | 32.039 | 47.064 | 2.212 | 1.00 | 8.16 | A |
| ATOM | 1937 | C | THR | A | 261 | 31.817 | 44.875 | 4.111 | 1.00 | 7.68 | A |
| ATOM | 1938 | O | THR | A | 261 | 30.870 | 44.096 | 3.992 | 1.00 | 8.41 | A |
| ATOM | 1939 | N | HIS | A | 262 | 31.922 | 45.758 | 5.100 | 1.00 | 6.93 | A |
| ATOM | 1940 | CA | HIS | A | 262 | 30.901 | 45.912 | 6.123 | 1.00 | 7.90 | A |
| ATOM | 1941 | CB | HIS | A | 262 | 31.519 | 45.801 | 7.522 | 1.00 | 8.17 | A |
| ATOM | 1942 | CG | HIS | A | 262 | 30.578 | 46.163 | 8.632 | 1.00 | 8.05 | A |
| ATOM | 1943 | CD2 | HIS | A | 262 | 30.160 | 47.368 | 9.091 | 1.00 | 8.34 | A |
| ATOM | 1944 | ND1 | HIS | A | 262 | 29.948 | 45.218 | 9.414 | 1.00 | 8.69 | A |
| ATOM | 1945 | CE1 | HIS | A | 262 | 29.185 | 45.825 | 10.308 | 1.00 | 8.74 | A |
| ATOM | 1946 | NE2 | HIS | A | 262 | 29.296 | 47.130 | 10.134 | 1.00 | 9.13 | A |
| ATOM | 1947 | C | HIS | A | 262 | 30.274 | 47.293 | 5.964 | 1.00 | 8.58 | A |
| ATOM | 1948 | O | HIS | A | 262 | 30.973 | 48.307 | 6.012 | 1.00 | 8.10 | A |
| ATOM | 1949 | N | MSE | A | 263 | 28.965 | 47.333 | 5.747 | 1.00 | 8.26 | A |
| ATOM | 1950 | CA | MSE | A | 263 | 28.257 | 48.600 | 5.633 | 1.00 | 9.29 | A |
| ATOM | 1951 | CB | MSE | A | 263 | 27.263 | 48.572 | 4.469 | 1.00 | 10.58 | A |
| ATOM | 1952 | CG | MSE | A | 263 | 26.474 | 49.869 | 4.305 | 1.00 | 10.98 | A |
| ATOM | 1953 | SE | MSE | A | 263 | 24.989 | 49.720 | 3.067 | 1.00 | 16.18 | A |
| ATOM | 1954 | CE | MSE | A | 263 | 25.986 | 49.431 | 1.449 | 1.00 | 13.27 | A |
| ATOM | 1955 | C | MSE | A | 263 | 27.493 | 48.822 | 6.935 | 1.00 | 9.90 | A |
| ATOM | 1956 | O | MSE | A | 263 | 26.734 | 47.949 | 7.372 | 1.00 | 10.15 | A |
| ATOM | 1957 | N | MSE | A | 264 | 27.709 | 49.973 | 7.567 | 1.00 | 9.48 | A |
| ATOM | 1958 | CA | MSE | A | 264 | 27.000 | 50.294 | 8.800 | 1.00 | 11.38 | A |
| ATOM | 1959 | CB | MSE | A | 264 | 27.599 | 51.544 | 9.434 | 1.00 | 13.97 | A |
| ATOM | 1960 | CG | MSE | A | 264 | 28.998 | 51.276 | 9.969 | 1.00 | 18.24 | A |
| ATOM | 1961 | SE | MSE | A | 264 | 29.922 | 52.842 | 10.605 | 1.00 | 30.43 | A |
| ATOM | 1962 | CE | MSE | A | 264 | 28.649 | 53.434 | 11.918 | 1.00 | 25.37 | A |
| ATOM | 1963 | C | MSE | A | 264 | 25.544 | 50.473 | 8.391 | 1.00 | 10.63 | A |
| ATOM | 1964 | O | MSE | A | 264 | 25.254 | 50.951 | 7.296 | 1.00 | 10.87 | A |
| ATOM | 1965 | N | PRO | A | 265 | 24.605 | 50.109 | 9.274 | 1.00 | 10.69 | A |
| ATOM | 1966 | CD | PRO | A | 265 | 24.819 | 49.543 | 10.620 | 1.00 | 10.66 | A |
| ATOM | 1967 | CA | PRO | A | 265 | 23.177 | 50.203 | 8.973 | 1.00 | 11.01 | A |
| ATOM | 1968 | CB | PRO | A | 265 | 22.610 | 49.052 | 9.785 | 1.00 | 10.51 | A |
| ATOM | 1969 | CG | PRO | A | 265 | 23.392 | 49.183 | 11.069 | 1.00 | 10.95 | A |
| ATOM | 1970 | C | PRO | A | 265 | 22.390 | 51.468 | 9.232 | 1.00 | 11.27 | A |
| ATOM | 1971 | O | PRO | A | 265 | 21.302 | 51.635 | 8.681 | 1.00 | 11.33 | A |
| ATOM | 1972 | N | PHE | A | 266 | 22.929 | 52.361 | 10.048 | 1.00 | 11.35 | A |
| ATOM | 1973 | CA | PHE | A | 266 | 22.177 | 53.540 | 10.421 | 1.00 | 11.37 | A |
| ATOM | 1974 | CB | PHE | A | 266 | 22.134 | 53.589 | 11.952 | 1.00 | 10.85 | A |
| ATOM | 1975 | CG | PHE | A | 266 | 21.602 | 52.310 | 12.573 | 1.00 | 10.84 | A |
| ATOM | 1976 | CD1 | PHE | A | 266 | 22.225 | 51.740 | 13.679 | 1.00 | 10.84 | A |
| ATOM | 1977 | CD2 | PHE | A | 266 | 20.487 | 51.669 | 12.031 | 1.00 | 11.07 | A |
| ATOM | 1978 | CE1 | PHE | A | 266 | 21.748 | 50.551 | 14.236 | 1.00 | 10.63 | A |
| ATOM | 1979 | CE2 | PHE | A | 266 | 20.001 | 50.481 | 12.580 | 1.00 | 10.14 | A |
| ATOM | 1980 | CZ | PHE | A | 266 | 20.633 | 49.921 | 13.682 | 1.00 | 9.88 | A |
| ATOM | 1981 | C | PHE | A | 266 | 22.564 | 54.885 | 9.812 | 1.00 | 11.36 | A |
| ATOM | 1982 | O | PHE | A | 266 | 23.495 | 54.988 | 9.017 | 1.00 | 11.87 | A |
| ATOM | 1983 | N | TYR | A | 267 | 21.812 | 55.907 | 10.203 | 1.00 | 11.40 | A |
| ATOM | 1984 | CA | TYR | A | 267 | 21.949 | 57.273 | 9.709 | 1.00 | 11.71 | A |
| ATOM | 1985 | CB | TYR | A | 267 | 20.814 | 58.099 | 10.319 | 1.00 | 12.66 | A |
| ATOM | 1986 | CG | TYR | A | 267 | 20.908 | 59.594 | 10.143 | 1.00 | 12.67 | A |
| ATOM | 1987 | CD1 | TYR | A | 267 | 20.575 | 60.204 | 8.934 | 1.00 | 13.51 | A |
| ATOM | 1988 | CE1 | TYR | A | 267 | 20.613 | 61.597 | 8.798 | 1.00 | 14.20 | A |
| ATOM | 1989 | CD2 | TYR | A | 267 | 21.288 | 60.406 | 11.211 | 1.00 | 13.73 | A |
| ATOM | 1990 | CE2 | TYR | A | 267 | 21.330 | 61.786 | 11.085 | 1.00 | 14.05 | A |
| ATOM | 1991 | CZ | TYR | A | 267 | 20.990 | 62.376 | 9.882 | 1.00 | 15.05 | A |
| ATOM | 1992 | OH | TYR | A | 267 | 21.017 | 63.747 | 9.784 | 1.00 | 16.91 | A |
| ATOM | 1993 | C | TYR | A | 267 | 23.284 | 57.987 | 9.916 | 1.00 | 11.75 | A |
| ATOM | 1994 | O | TYR | A | 267 | 23.685 | 58.807 | 9.085 | 1.00 | 11.62 | A |
| ATOM | 1995 | N | SER | A | 268 | 23.973 | 57.684 | 11.008 | 1.00 | 11.53 | A |
| ATOM | 1996 | CA | SER | A | 268 | 25.240 | 58.346 | 11.299 | 1.00 | 10.96 | A |
| ATOM | 1997 | CB | SER | A | 268 | 24.975 | 59.572 | 12.178 | 1.00 | 11.47 | A |
| ATOM | 1998 | OG | SER | A | 268 | 26.180 | 60.184 | 12.600 | 1.00 | 13.03 | A |
| ATOM | 1999 | C | SER | A | 268 | 26.236 | 57.432 | 11.992 | 1.00 | 10.90 | A |
| ATOM | 2000 | O | SER | A | 268 | 25.891 | 56.329 | 12.417 | 1.00 | 10.38 | A |
| ATOM | 2001 | N | TYR | A | 269 | 27.480 | 57.892 | 12.094 | 1.00 | 10.75 | A |
| ATOM | 2002 | CA | TYR | A | 269 | 28.514 | 57.127 | 12.775 | 1.00 | 10.42 | A |
| ATOM | 2003 | CB | TYR | A | 269 | 29.872 | 57.282 | 12.069 | 1.00 | 10.68 | A |
| ATOM | 2004 | CG | TYR | A | 269 | 30.306 | 58.714 | 11.844 | 1.00 | 10.71 | A |
| ATOM | 2005 | CD1 | TYR | A | 269 | 30.698 | 59.529 | 12.909 | 1.00 | 10.21 | A |
| ATOM | 2006 | CE1 | TYR | A | 269 | 31.055 | 60.864 | 12.702 | 1.00 | 11.28 | A |
| ATOM | 2007 | CD2 | TYR | A | 269 | 30.288 | 59.265 | 10.565 | 1.00 | 10.99 | A |
| ATOM | 2008 | CE2 | TYR | A | 269 | 30.642 | 60.595 | 10.348 | 1.00 | 10.95 | A |
| ATOM | 2009 | CZ | TYR | A | 269 | 31.020 | 61.387 | 11.420 | 1.00 | 11.31 | A |
| ATOM | 2010 | OH | TYR | A | 269 | 31.331 | 62.711 | 11.209 | 1.00 | 11.28 | A |
| ATOM | 2011 | C | TYR | A | 269 | 28.627 | 57.587 | 14.227 | 1.00 | 10.96 | A |
| ATOM | 2012 | O | TYR | A | 269 | 29.444 | 57.060 | 14.981 | 1.00 | 10.70 | A |
| ATOM | 2013 | N | ASP | A | 270 | 27.816 | 58.570 | 14.625 | 1.00 | 10.74 | A |
| ATOM | 2014 | CA | ASP | A | 270 | 27.879 | 59.042 | 16.006 | 1.00 | 10.41 | A |
| ATOM | 2015 | CB | ASP | A | 270 | 27.196 | 60.416 | 16.178 | 1.00 | 11.57 | A |
| ATOM | 2016 | CG | ASP | A | 270 | 25.710 | 60.404 | 15.861 | 1.00 | 11.76 | A |
| ATOM | 2017 | OD1 | ASP | A | 270 | 25.095 | 59.320 | 15.799 | 1.00 | 12.51 | A |
| ATOM | 2018 | OD2 | ASP | A | 270 | 25.150 | 61.513 | 15.693 | 1.00 | 14.61 | A |
| ATOM | 2019 | C | ASP | A | 270 | 27.292 | 57.998 | 16.950 | 1.00 | 10.60 | A |
| ATOM | 2020 | O | ASP | A | 270 | 26.684 | 57.020 | 16.511 | 1.00 | 10.31 | A |
| ATOM | 2021 | N | ILE | A | 271 | 27.492 | 58.192 | 18.246 | 1.00 | 10.34 | A |
| ATOM | 2022 | CA | ILE | A | 271 | 27.027 | 57.212 | 19.214 | 1.00 | 10.55 | A |
| ATOM | 2023 | CB | ILE | A | 271 | 27.536 | 57.588 | 20.622 | 1.00 | 10.92 | A |
| ATOM | 2024 | CG2 | ILE | A | 271 | 27.035 | 56.589 | 21.659 | 1.00 | 10.05 | A |
| ATOM | 2025 | CG1 | ILE | A | 271 | 29.071 | 57.563 | 20.602 | 1.00 | 11.08 | A |
| ATOM | 2026 | CD1 | ILE | A | 271 | 29.745 | 58.096 | 21.846 | 1.00 | 11.63 | A |
| ATOM | 2027 | C | ILE | A | 271 | 25.518 | 56.956 | 19.185 | 1.00 | 10.58 | A |
| ATOM | 2028 | O | ILE | A | 271 | 25.083 | 55.807 | 19.260 | 1.00 | 10.41 | A |
| ATOM | 2029 | N | PRO | A | 272 | 24.700 | 58.011 | 19.057 | 1.00 | 10.98 | A |
| ATOM | 2030 | CD | PRO | A | 272 | 24.985 | 59.453 | 19.156 | 1.00 | 11.33 | A |
| ATOM | 2031 | CA | PRO | A | 272 | 23.255 | 57.758 | 19.022 | 1.00 | 10.92 | A |
| ATOM | 2032 | CB | PRO | A | 272 | 22.665 | 59.160 | 18.878 | 1.00 | 11.04 | A |
| ATOM | 2033 | CG | PRO | A | 272 | 23.659 | 60.016 | 19.626 | 1.00 | 11.74 | A |
| ATOM | 2034 | C | PRO | A | 272 | 22.826 | 56.829 | 17.876 | 1.00 | 11.69 | A |
| ATOM | 2035 | O | PRO | A | 272 | 21.785 | 56.180 | 17.959 | 1.00 | 11.90 | A |
| ATOM | 2036 | N | HIS | A | 273 | 23.629 | 56.750 | 16.815 | 1.00 | 10.74 | A |
| ATOM | 2037 | CA | HIS | A | 273 | 23.278 | 55.908 | 15.672 | 1.00 | 10.78 | A |
| ATOM | 2038 | CB | HIS | A | 273 | 23.224 | 56.767 | 14.405 | 1.00 | 10.02 | A |
| ATOM | 2039 | CG | HIS | A | 273 | 22.235 | 57.887 | 14.491 | 1.00 | 11.26 | A |
| ATOM | 2040 | CD2 | HIS | A | 273 | 22.380 | 59.169 | 14.901 | 1.00 | 9.55 | A |
| ATOM | 2041 | ND1 | HIS | A | 273 | 20.897 | 57.727 | 14.202 | 1.00 | 12.40 | A |
| ATOM | 2042 | CE1 | HIS | A | 273 | 20.261 | 58.862 | 14.433 | 1.00 | 9.52 | A |
| ATOM | 2043 | NE2 | HIS | A | 273 | 21.137 | 59.753 | 14.859 | 1.00 | 13.79 | A |
| ATOM | 2044 | C | HIS | A | 273 | 24.195 | 54.711 | 15.456 | 1.00 | 11.09 | A |
| ATOM | 2045 | O | HIS | A | 273 | 24.260 | 54.165 | 14.353 | 1.00 | 11.15 | A |
| ATOM | 2046 | N | THR | A | 274 | 24.891 | 54.287 | 16.508 | 1.00 | 11.34 | A |
| ATOM | 2047 | CA | THR | A | 274 | 25.782 | 53.140 | 16.385 | 1.00 | 11.09 | A |
| ATOM | 2048 | CB | THR | A | 274 | 27.269 | 53.578 | 16.377 | 1.00 | 12.31 | A |
| ATOM | 2049 | OG1 | THR | A | 274 | 27.513 | 54.504 | 17.442 | 1.00 | 11.45 | A |
| ATOM | 2050 | CG2 | THR | A | 274 | 27.620 | 54.224 | 15.053 | 1.00 | 11.39 | A |
| ATOM | 2051 | C | THR | A | 274 | 25.598 | 52.033 | 17.425 | 1.00 | 11.88 | A |
| ATOM | 2052 | O | THR | A | 274 | 26.215 | 50.976 | 17.311 | 1.00 | 12.79 | A |
| ATOM | 2053 | N | CYS | A | 275 | 24.752 | 52.250 | 18.429 | 1.00 | 11.97 | A |
| ATOM | 2054 | CA | CYS | A | 275 | 24.543 | 51.214 | 19.445 | 1.00 | 12.65 | A |
| ATOM | 2055 | C | CYS | A | 275 | 23.361 | 50.321 | 19.105 | 1.00 | 12.65 | A |
| ATOM | 2056 | O | CYS | A | 275 | 23.291 | 49.169 | 19.536 | 1.00 | 12.89 | A |
| ATOM | 2057 | CB | CYS | A | 275 | 24.315 | 51.845 | 20.824 | 1.00 | 13.02 | A |
| ATOM | 2058 | SG | CYS | A | 275 | 22.586 | 51.943 | 21.413 | 1.00 | 13.25 | A |
| ATOM | 2059 | N | GLY | A | 276 | 22.433 | 50.865 | 18.329 | 1.00 | 12.85 | A |
| ATOM | 2060 | CA | GLY | A | 276 | 21.243 | 50.130 | 17.954 | 1.00 | 12.82 | A |
| ATOM | 2061 | C | GLY | A | 276 | 20.329 | 51.021 | 17.139 | 1.00 | 12.23 | A |
| ATOM | 2062 | O | GLY | A | 276 | 20.704 | 52.149 | 16.813 | 1.00 | 12.13 | A |
| ATOM | 2063 | N | PRO | A | 277 | 19.113 | 50.558 | 16.812 | 1.00 | 11.60 | A |
| ATOM | 2064 | CD | PRO | A | 277 | 18.595 | 49.229 | 17.187 | 1.00 | 11.96 | A |
| ATOM | 2065 | CA | PRO | A | 277 | 18.123 | 51.294 | 16.021 | 1.00 | 12.04 | A |
| ATOM | 2066 | CB | PRO | A | 277 | 17.119 | 50.208 | 15.650 | 1.00 | 12.10 | A |
| ATOM | 2067 | CG | PRO | A | 277 | 17.116 | 49.352 | 16.885 | 1.00 | 10.84 | A |
| ATOM | 2068 | C | PRO | A | 277 | 17.435 | 52.502 | 16.652 | 1.00 | 11.83 | A |
| ATOM | 2069 | O | PRO | A | 277 | 16.844 | 53.310 | 15.938 | 1.00 | 12.62 | A |
| ATOM | 2070 | N | ASP | A | 278 | 17.500 | 52.628 | 17.973 | 1.00 | 12.62 | A |
| ATOM | 2071 | CA | ASP | A | 278 | 16.834 | 53.741 | 18.650 | 1.00 | 13.37 | A |
| ATOM | 2072 | CB | ASP | A | 278 | 15.969 | 53.213 | 19.799 | 1.00 | 13.54 | A |
| ATOM | 2073 | CG | ASP | A | 278 | 15.045 | 54.274 | 20.368 | 1.00 | 14.93 | A |
| ATOM | 2074 | OD1 | ASP | A | 278 | 15.194 | 55.456 | 20.004 | 1.00 | 15.72 | A |
| ATOM | 2075 | OD2 | ASP | A | 278 | 14.168 | 53.925 | 21.185 | 1.00 | 15.70 | A |
| ATOM | 2076 | C | ASP | A | 278 | 17.814 | 54.773 | 19.194 | 1.00 | 13.23 | A |
| ATOM | 2077 | O | ASP | A | 278 | 18.426 | 54.568 | 20.241 | 1.00 | 13.99 | A |
| ATOM | 2078 | N | PRO | A | 279 | 17.962 | 55.908 | 18.493 | 1.00 | 13.69 | A |
| ATOM | 2079 | CD | PRO | A | 279 | 17.262 | 56.315 | 17.263 | 1.00 | 13.30 | A |
| ATOM | 2080 | CA | PRO | A | 279 | 18.886 | 56.952 | 18.944 | 1.00 | 13.86 | A |
| ATOM | 2081 | CB | PRO | A | 279 | 18.834 | 57.975 | 17.809 | 1.00 | 13.40 | A |
| ATOM | 2082 | CG | PRO | A | 279 | 17.448 | 57.815 | 17.269 | 1.00 | 13.26 | A |
| ATOM | 2083 | C | PRO | A | 279 | 18.537 | 57.555 | 20.302 | 1.00 | 14.31 | A |
| ATOM | 2084 | O | PRO | A | 279 | 19.415 | 58.042 | 21.011 | 1.00 | 14.71 | A |
| ATOM | 2085 | N | LYS | A | 280 | 17.260 | 57.518 | 20.669 | 1.00 | 15.00 | A |
| ATOM | 2086 | CA | LYS | A | 280 | 16.841 | 58.061 | 21.957 | 1.00 | 16.46 | A |
| ATOM | 2087 | CB | LYS | A | 280 | 15.316 | 58.029 | 22.084 | 1.00 | 17.95 | A |
| ATOM | 2088 | CG | LYS | A | 280 | 14.798 | 58.566 | 23.413 | 1.00 | 20.74 | A |
| ATOM | 2089 | CD | LYS | A | 280 | 13.277 | 58.545 | 23.472 | 1.00 | 23.13 | A |
| ATOM | 2090 | CE | LYS | A | 280 | 12.733 | 57.124 | 23.426 | 1.00 | 25.35 | A |
| ATOM | 2091 | NZ | LYS | A | 280 | 13.197 | 56.302 | 24.586 | 1.00 | 27.48 | A |
| ATOM | 2092 | C | LYS | A | 280 | 17.469 | 57.244 | 23.080 | 1.00 | 16.31 | A |
| ATOM | 2093 | O | LYS | A | 280 | 17.770 | 57.771 | 24.152 | 1.00 | 17.31 | A |
| ATOM | 2094 | N | VAL | A | 281 | 17.669 | 55.953 | 22.828 | 1.00 | 15.76 | A |
| ATOM | 2095 | CA | VAL | A | 281 | 18.276 | 55.067 | 23.814 | 1.00 | 14.99 | A |
| ATOM | 2096 | CB | VAL | A | 281 | 17.837 | 53.598 | 23.602 | 1.00 | 15.83 | A |
| ATOM | 2097 | CG1 | VAL | A | 281 | 18.565 | 52.687 | 24.585 | 1.00 | 15.97 | A |
| ATOM | 2098 | CG2 | VAL | A | 281 | 16.328 | 53.475 | 23.775 | 1.00 | 15.53 | A |
| ATOM | 2099 | C | VAL | A | 281 | 19.797 | 55.132 | 23.720 | 1.00 | 14.77 | A |
| ATOM | 2100 | O | VAL | A | 281 | 20.488 | 55.285 | 24.725 | 1.00 | 13.51 | A |
| ATOM | 2101 | N | CYS | A | 282 | 20.317 | 55.021 | 22.503 | 1.00 | 13.87 | A |
| ATOM | 2102 | CA | CYS | A | 282 | 21.759 | 55.056 | 22.296 | 1.00 | 13.58 | A |
| ATOM | 2103 | C | CYS | A | 282 | 22.402 | 56.340 | 22.804 | 1.00 | 13.26 | A |
| ATOM | 2104 | O | CYS | A | 282 | 23.524 | 56.320 | 23.308 | 1.00 | 12.92 | A |
| ATOM | 2105 | CB | CYS | A | 282 | 22.086 | 54.884 | 20.814 | 1.00 | 13.80 | A |
| ATOM | 2106 | SG | CYS | A | 282 | 21.707 | 53.243 | 20.122 | 1.00 | 13.83 | A |
| ATOM | 2107 | N | CYS | A | 283 | 21.695 | 57.458 | 22.673 | 1.00 | 12.97 | A |
| ATOM | 2108 | CA | CYS | A | 283 | 22.241 | 58.727 | 23.130 | 1.00 | 13.37 | A |
| ATOM | 2109 | C | CYS | A | 283 | 22.543 | 58.688 | 24.624 | 1.00 | 13.10 | A |
| ATOM | 2110 | O | CYS | A | 283 | 23.435 | 59.387 | 25.102 | 1.00 | 12.57 | A |
| ATOM | 2111 | CB | CYS | A | 283 | 21.274 | 59.876 | 22.829 | 1.00 | 15.13 | A |
| ATOM | 2112 | SG | CYS | A | 283 | 22.121 | 61.486 | 22.906 | 1.00 | 17.02 | A |
| ATOM | 2113 | N | GLN | A | 284 | 21.804 | 57.860 | 25.359 | 1.00 | 12.91 | A |
| ATOM | 2114 | CA | GLN | A | 284 | 22.003 | 57.740 | 26.797 | 1.00 | 13.67 | A |
| ATOM | 2115 | CB | GLN | A | 284 | 20.821 | 57.006 | 27.433 | 1.00 | 14.14 | A |
| ATOM | 2116 | CG | GLN | A | 284 | 19.496 | 57.719 | 27.260 | 1.00 | 15.49 | A |
| ATOM | 2117 | CD | GLN | A | 284 | 18.338 | 56.905 | 27.790 | 1.00 | 15.23 | A |
| ATOM | 2118 | OE1 | GLN | A | 284 | 18.315 | 56.530 | 28.962 | 1.00 | 17.22 | A |
| ATOM | 2119 | NE2 | GLN | A | 284 | 17.372 | 56.622 | 26.929 | 1.00 | 16.85 | A |
| ATOM | 2120 | C | GLN | A | 284 | 23.292 | 57.009 | 27.136 | 1.00 | 13.15 | A |
| ATOM | 2121 | O | GLN | A | 284 | 23.675 | 56.925 | 28.302 | 1.00 | 13.79 | A |
| ATOM | 2122 | N | PHE | A | 285 | 23.960 | 56.479 | 26.116 | 1.00 | 12.56 | A |
| ATOM | 2123 | CA | PHE | A | 285 | 25.201 | 55.759 | 26.336 | 1.00 | 12.06 | A |
| ATOM | 2124 | CB | PHE | A | 285 | 25.045 | 54.319 | 25.839 | 1.00 | 12.17 | A |
| ATOM | 2125 | CG | PHE | A | 285 | 24.074 | 53.520 | 26.671 | 1.00 | 12.29 | A |
| ATOM | 2126 | CD1 | PHE | A | 285 | 24.485 | 52.917 | 27.858 | 1.00 | 12.76 | A |
| ATOM | 2127 | CD2 | PHE | A | 285 | 22.728 | 53.453 | 26.317 | 1.00 | 13.09 | A |
| ATOM | 2128 | CE1 | PHE | A | 285 | 23.565 | 52.262 | 28.686 | 1.00 | 13.11 | A |
| ATOM | 2129 | CE2 | PHE | A | 285 | 21.800 | 52.803 | 27.136 | 1.00 | 14.38 | A |
| ATOM | 2130 | CZ | PHE | A | 285 | 22.221 | 52.208 | 28.324 | 1.00 | 13.55 | A |
| ATOM | 2131 | C | PHE | A | 285 | 26.414 | 56.471 | 25.748 | 1.00 | 11.90 | A |
| ATOM | 2132 | O | PHE | A | 285 | 27.469 | 55.876 | 25.521 | 1.00 | 11.81 | A |
| ATOM | 2133 | N | ASP | A | 286 | 26.233 | 57.764 | 25.504 | 1.00 | 11.70 | A |
| ATOM | 2134 | CA | ASP | A | 286 | 27.303 | 58.639 | 25.038 | 1.00 | 11.79 | A |
| ATOM | 2135 | CB | ASP | A | 286 | 26.844 | 59.533 | 23.889 | 1.00 | 11.90 | A |
| ATOM | 2136 | CG | ASP | A | 286 | 27.944 | 60.464 | 23.414 | 1.00 | 11.44 | A |
| ATOM | 2137 | OD1 | ASP | A | 286 | 28.966 | 60.569 | 24.128 | 1.00 | 12.21 | A |
| ATOM | 2138 | OD2 | ASP | A | 286 | 27.790 | 61.092 | 22.347 | 1.00 | 11.77 | A |
| ATOM | 2139 | C | ASP | A | 286 | 27.497 | 59.475 | 26.298 | 1.00 | 11.97 | A |
| ATOM | 2140 | O | ASP | A | 286 | 26.840 | 60.497 | 26.488 | 1.00 | 11.60 | A |
| ATOM | 2141 | N | PHE | A | 287 | 28.395 | 59.028 | 27.165 | 1.00 | 11.38 | A |
| ATOM | 2142 | CA | PHE | A | 287 | 28.603 | 59.697 | 28.437 | 1.00 | 12.39 | A |
| ATOM | 2143 | CB | PHE | A | 287 | 29.377 | 58.764 | 29.374 | 1.00 | 12.11 | A |
| ATOM | 2144 | CG | PHE | A | 287 | 28.648 | 57.473 | 29.651 | 1.00 | 12.28 | A |
| ATOM | 2145 | CD1 | PHE | A | 287 | 28.821 | 56.366 | 28.826 | 1.00 | 12.95 | A |
| ATOM | 2146 | CD2 | PHE | A | 287 | 27.717 | 57.397 | 30.683 | 1.00 | 13.46 | A |
| ATOM | 2147 | CE1 | PHE | A | 287 | 28.076 | 55.206 | 29.020 | 1.00 | 13.22 | A |
| ATOM | 2148 | CE2 | PHE | A | 287 | 26.964 | 56.239 | 30.885 | 1.00 | 12.89 | A |
| ATOM | 2149 | CZ | PHE | A | 287 | 27.143 | 55.141 | 30.049 | 1.00 | 13.07 | A |
| ATOM | 2150 | C | PHE | A | 287 | 29.207 | 61.091 | 28.414 | 1.00 | 13.59 | A |
| ATOM | 2151 | O | PHE | A | 287 | 29.405 | 61.695 | 29.465 | 1.00 | 14.01 | A |
| ATOM | 2152 | N | LYS | A | 288 | 29.482 | 61.620 | 27.227 | 1.00 | 14.45 | A |
| ATOM | 2153 | CA | LYS | A | 288 | 30.023 | 62.970 | 27.149 | 1.00 | 15.44 | A |
| ATOM | 2154 | CB | LYS | A | 288 | 30.933 | 63.128 | 25.924 | 1.00 | 15.21 | A |
| ATOM | 2155 | CG | LYS | A | 288 | 31.689 | 64.457 | 25.905 | 1.00 | 14.15 | A |
| ATOM | 2156 | CD | LYS | A | 288 | 32.592 | 64.586 | 24.684 | 1.00 | 13.96 | A |
| ATOM | 2157 | CE | LYS | A | 288 | 33.413 | 65.868 | 24.753 | 1.00 | 13.29 | A |
| ATOM | 2158 | NZ | LYS | A | 288 | 34.359 | 66.002 | 23.603 | 1.00 | 14.76 | A |
| ATOM | 2159 | C | LYS | A | 288 | 28.866 | 63.968 | 27.060 | 1.00 | 16.58 | A |
| ATOM | 2160 | O | LYS | A | 288 | 29.080 | 65.176 | 27.059 | 1.00 | 16.11 | A |
| ATOM | 2161 | N | ARG | A | 289 | 27.637 | 63.461 | 27.007 | 1.00 | 17.84 | A |
| ATOM | 2162 | CA | ARG | A | 289 | 26.471 | 64.335 | 26.894 | 1.00 | 20.20 | A |
| ATOM | 2163 | CB | ARG | A | 289 | 25.498 | 63.776 | 25.852 | 1.00 | 18.53 | A |
| ATOM | 2164 | CG | ARG | A | 289 | 26.110 | 63.516 | 24.487 | 1.00 | 16.64 | A |
| ATOM | 2165 | CD | ARG | A | 289 | 25.021 | 63.230 | 23.477 | 1.00 | 15.80 | A |
| ATOM | 2166 | NE | ARG | A | 289 | 25.545 | 62.852 | 22.168 | 1.00 | 13.90 | A |
| ATOM | 2167 | CZ | ARG | A | 289 | 25.094 | 63.347 | 21.021 | 1.00 | 14.07 | A |
| ATOM | 2168 | NH1 | ARG | A | 289 | 24.119 | 64.248 | 21.023 | 1.00 | 14.29 | A |
| ATOM | 2169 | NH2 | ARG | A | 289 | 25.603 | 62.931 | 19.871 | 1.00 | 13.78 | A |
| ATOM | 2170 | C | ARG | A | 289 | 25.701 | 64.571 | 28.193 | 1.00 | 23.25 | A |
| ATOM | 2171 | O | ARG | A | 289 | 24.473 | 64.635 | 28.174 | 1.00 | 23.38 | A |
| ATOM | 2172 | N | MSE | A | 290 | 26.398 | 64.717 | 29.314 | 1.00 | 27.14 | A |
| ATOM | 2173 | CA | MSE | A | 290 | 25.691 | 64.930 | 30.572 | 1.00 | 30.56 | A |
| ATOM | 2174 | CB | MSE | A | 290 | 26.309 | 64.067 | 31.681 | 1.00 | 33.87 | A |
| ATOM | 2175 | CG | MSE | A | 290 | 26.223 | 62.573 | 31.377 | 1.00 | 37.48 | A |
| ATOM | 2176 | SE | MSE | A | 290 | 26.343 | 61.383 | 32.902 | 1.00 | 42.06 | A |
| ATOM | 2177 | CE | MSE | A | 290 | 28.253 | 61.093 | 32.905 | 1.00 | 40.46 | A |
| ATOM | 2178 | C | MSE | A | 290 | 25.578 | 66.389 | 31.019 | 1.00 | 30.92 | A |
| ATOM | 2179 | O | MSE | A | 290 | 24.852 | 66.690 | 31.971 | 1.00 | 31.50 | A |
| ATOM | 2180 | N | GLY | A | 291 | 26.278 | 67.294 | 30.338 | 1.00 | 30.12 | A |
| ATOM | 2181 | CA | GLY | A | 291 | 26.182 | 68.701 | 30.698 | 1.00 | 28.34 | A |
| ATOM | 2182 | C | GLY | A | 291 | 27.455 | 69.531 | 30.733 | 1.00 | 27.01 | A |
| ATOM | 2183 | O | GLY | A | 291 | 27.560 | 70.545 | 30.040 | 1.00 | 27.30 | A |
| ATOM | 2184 | N | SER | A | 292 | 28.422 | 69.108 | 31.541 | 1.00 | 24.80 | A |
| ATOM | 2185 | CA | SER | A | 292 | 29.682 | 69.831 | 31.686 | 1.00 | 21.72 | A |
| ATOM | 2186 | CB | SER | A | 292 | 30.552 | 69.152 | 32.744 | 1.00 | 22.53 | A |
| ATOM | 2187 | OG | SER | A | 292 | 30.886 | 67.833 | 32.350 | 1.00 | 22.82 | A |
| ATOM | 2188 | C | SER | A | 292 | 30.488 | 69.984 | 30.396 | 1.00 | 20.05 | A |
| ATOM | 2189 | O | SER | A | 292 | 31.362 | 70.848 | 30.311 | 1.00 | 18.67 | A |
| ATOM | 2190 | N | PHE | A | 293 | 30.202 | 69.151 | 29.399 | 1.00 | 18.19 | A |
| ATOM | 2191 | CA | PHE | A | 293 | 30.919 | 69.220 | 28.128 | 1.00 | 17.46 | A |
| ATOM | 2192 | CB | PHE | A | 293 | 31.136 | 67.819 | 27.546 | 1.00 | 16.98 | A |
| ATOM | 2193 | CG | PHE | A | 293 | 32.049 | 66.956 | 28.361 | 1.00 | 15.98 | A |
| ATOM | 2194 | CD1 | PHE | A | 293 | 31.535 | 66.066 | 29.295 | 1.00 | 16.38 | A |
| ATOM | 2195 | CD2 | PHE | A | 293 | 33.427 | 67.032 | 28.191 | 1.00 | 15.50 | A |
| ATOM | 2196 | CE1 | PHE | A | 293 | 32.383 | 65.258 | 30.050 | 1.00 | 16.47 | A |
| ATOM | 2197 | CE2 | PHE | A | 293 | 34.284 | 66.230 | 28.939 | 1.00 | 16.29 | A |
| ATOM | 2198 | CZ | PHE | A | 293 | 33.760 | 65.342 | 29.870 | 1.00 | 16.44 | A |
| ATOM | 2199 | C | PHE | A | 293 | 30.197 | 70.070 | 27.095 | 1.00 | 17.55 | A |
| ATOM | 2200 | O | PHE | A | 293 | 30.658 | 70.200 | 25.960 | 1.00 | 16.94 | A |
| ATOM | 2201 | N | GLY | A | 294 | 29.064 | 70.644 | 27.484 | 1.00 | 17.08 | A |
| ATOM | 2202 | CA | GLY | A | 294 | 28.314 | 71.471 | 26.557 | 1.00 | 17.66 | A |
| ATOM | 2203 | C | GLY | A | 294 | 27.592 | 70.671 | 25.489 | 1.00 | 17.59 | A |
| ATOM | 2204 | O | GLY | A | 294 | 27.302 | 71.181 | 24.407 | 1.00 | 18.62 | A |
| ATOM | 2205 | N | LEU | A | 295 | 27.309 | 69.409 | 25.788 | 1.00 | 17.46 | A |
| ATOM | 2206 | CA | LEU | A | 295 | 26.604 | 68.543 | 24.854 | 1.00 | 17.07 | A |
| ATOM | 2207 | CB | LEU | A | 295 | 27.514 | 67.400 | 24.392 | 1.00 | 17.17 | A |
| ATOM | 2208 | CG | LEU | A | 295 | 28.783 | 67.787 | 23.625 | 1.00 | 17.32 | A |
| ATOM | 2209 | CD1 | LEU | A | 295 | 29.647 | 66.554 | 23.406 | 1.00 | 17.83 | A |
| ATOM | 2210 | CD2 | LEU | A | 295 | 28.405 | 68.418 | 22.290 | 1.00 | 17.46 | A |
| ATOM | 2211 | C | LEU | A | 295 | 25.379 | 67.972 | 25.552 | 1.00 | 17.33 | A |
| ATOM | 2212 | O | LEU | A | 295 | 25.344 | 67.869 | 26.777 | 1.00 | 18.00 | A |
| ATOM | 2213 | N | SER | A | 296 | 24.371 | 67.610 | 24.769 | 1.00 | 17.40 | A |
| ATOM | 2214 | CA | SER | A | 296 | 23.147 | 67.047 | 25.319 | 1.00 | 18.18 | A |
| ATOM | 2215 | CB | SER | A | 296 | 22.162 | 68.167 | 25.679 | 1.00 | 18.23 | A |
| ATOM | 2216 | OG | SER | A | 296 | 21.859 | 68.973 | 24.553 | 1.00 | 19.31 | A |
| ATOM | 2217 | C | SER | A | 296 | 22.518 | 66.101 | 24.310 | 1.00 | 18.33 | A |
| ATOM | 2218 | O | SER | A | 296 | 23.031 | 65.931 | 23.203 | 1.00 | 17.91 | A |
| ATOM | 2219 | N | CYS | A | 297 | 21.415 | 65.476 | 24.708 | 1.00 | 18.81 | A |
| ATOM | 2220 | CA | CYS | A | 297 | 20.698 | 64.549 | 23.842 | 1.00 | 19.38 | A |
| ATOM | 2221 | C | CYS | A | 297 | 19.429 | 65.212 | 23.322 | 1.00 | 19.86 | A |
| ATOM | 2222 | O | CYS | A | 297 | 18.542 | 65.562 | 24.098 | 1.00 | 20.21 | A |
| ATOM | 2223 | CB | CYS | A | 297 | 20.355 | 63.268 | 24.609 | 1.00 | 19.22 | A |
| ATOM | 2224 | SG | CYS | A | 297 | 21.786 | 62.162 | 24.797 | 1.00 | 20.14 | A |
| ATOM | 2225 | N | PRO | A | 298 | 19.332 | 65.400 | 21.997 | 1.00 | 19.98 | A |
| ATOM | 2226 | CD | PRO | A | 298 | 20.330 | 65.059 | 20.966 | 1.00 | 20.03 | A |
| ATOM | 2227 | CA | PRO | A | 298 | 18.154 | 66.031 | 21.395 | 1.00 | 20.06 | A |
| ATOM | 2228 | CB | PRO | A | 298 | 18.557 | 66.189 | 19.929 | 1.00 | 20.76 | A |
| ATOM | 2229 | CG | PRO | A | 298 | 19.494 | 65.041 | 19.709 | 1.00 | 20.96 | A |
| ATOM | 2230 | C | PRO | A | 298 | 16.863 | 65.237 | 21.571 | 1.00 | 19.89 | A |
| ATOM | 2231 | O | PRO | A | 298 | 15.772 | 65.778 | 21.399 | 1.00 | 20.14 | A |
| ATOM | 2232 | N | TRP | A | 299 | 16.991 | 63.961 | 21.921 | 1.00 | 19.23 | A |
| ATOM | 2233 | CA | TRP | A | 299 | 15.827 | 63.105 | 22.124 | 1.00 | 19.96 | A |
| ATOM | 2234 | CB | TRP | A | 299 | 16.196 | 61.644 | 21.833 | 1.00 | 19.20 | A |
| ATOM | 2235 | CG | TRP | A | 299 | 16.531 | 61.437 | 20.381 | 1.00 | 18.35 | A |
| ATOM | 2236 | CD2 | TRP | A | 299 | 17.834 | 61.499 | 19.781 | 1.00 | 18.02 | A |
| ATOM | 2237 | CE2 | TRP | A | 299 | 17.662 | 61.346 | 18.388 | 1.00 | 18.44 | A |
| ATOM | 2238 | CE3 | TRP | A | 299 | 19.130 | 61.674 | 20.286 | 1.00 | 18.03 | A |
| ATOM | 2239 | CD1 | TRP | A | 299 | 15.650 | 61.248 | 19.355 | 1.00 | 18.36 | A |
| ATOM | 2240 | NE1 | TRP | A | 299 | 16.321 | 61.193 | 18.155 | 1.00 | 19.07 | A |
| ATOM | 2241 | CZ2 | TRP | A | 299 | 18.738 | 61.363 | 17.492 | 1.00 | 18.17 | A |
| ATOM | 2242 | CZ3 | TRP | A | 299 | 20.200 | 61.691 | 19.396 | 1.00 | 18.03 | A |
| ATOM | 2243 | CH2 | TRP | A | 299 | 19.994 | 61.536 | 18.014 | 1.00 | 17.94 | A |
| ATOM | 2244 | C | TRP | A | 299 | 15.247 | 63.270 | 23.530 | 1.00 | 21.12 | A |
| ATOM | 2245 | O | TRP | A | 299 | 14.351 | 62.531 | 23.937 | 1.00 | 21.04 | A |
| ATOM | 2246 | N | LYS | A | 300 | 15.783 | 64.242 | 24.265 | 1.00 | 22.23 | A |
| ATOM | 2247 | CA | LYS | A | 300 | 15.306 | 64.591 | 25.606 | 1.00 | 23.37 | A |
| ATOM | 2248 | CB | LYS | A | 300 | 13.795 | 64.829 | 25.552 | 1.00 | 25.08 | A |
| ATOM | 2249 | CG | LYS | A | 300 | 13.400 | 65.978 | 24.643 | 1.00 | 26.56 | A |
| ATOM | 2250 | CD | LYS | A | 300 | 11.890 | 66.085 | 24.505 | 1.00 | 28.74 | A |
| ATOM | 2251 | CE | LYS | A | 300 | 11.497 | 67.267 | 23.632 | 1.00 | 29.78 | A |
| ATOM | 2252 | NZ | LYS | A | 300 | 11.893 | 68.565 | 24.247 | 1.00 | 31.18 | A |
| ATOM | 2253 | C | LYS | A | 300 | 15.628 | 63.703 | 26.806 | 1.00 | 23.29 | A |
| ATOM | 2254 | O | LYS | A | 300 | 15.248 | 64.032 | 27.930 | 1.00 | 23.45 | A |
| ATOM | 2255 | N | VAL | A | 301 | 16.304 | 62.579 | 26.591 | 1.00 | 22.63 | A |
| ATOM | 2256 | CA | VAL | A | 301 | 16.669 | 61.717 | 27.709 | 1.00 | 22.92 | A |
| ATOM | 2257 | CB | VAL | A | 301 | 16.100 | 60.282 | 27.553 | 1.00 | 23.50 | A |
| ATOM | 2258 | CG1 | VAL | A | 301 | 14.582 | 60.328 | 27.567 | 1.00 | 24.41 | A |
| ATOM | 2259 | CG2 | VAL | A | 301 | 16.582 | 59.652 | 26.267 | 1.00 | 24.91 | A |
| ATOM | 2260 | C | VAL | A | 301 | 18.191 | 61.686 | 27.783 | 1.00 | 22.38 | A |
| ATOM | 2261 | O | VAL | A | 301 | 18.858 | 61.129 | 26.913 | 1.00 | 22.87 | A |
| ATOM | 2262 | N | PRO | A | 302 | 18.761 | 62.302 | 28.827 | 1.00 | 21.66 | A |
| ATOM | 2263 | CD | PRO | A | 302 | 18.069 | 63.041 | 29.901 | 1.00 | 21.57 | A |
| ATOM | 2264 | CA | PRO | A | 302 | 20.211 | 62.357 | 29.013 | 1.00 | 21.07 | A |
| ATOM | 2265 | CB | PRO | A | 302 | 20.375 | 63.492 | 30.014 | 1.00 | 21.45 | A |
| ATOM | 2266 | CG | PRO | A | 302 | 19.184 | 63.292 | 30.899 | 1.00 | 21.82 | A |
| ATOM | 2267 | C | PRO | A | 302 | 20.851 | 61.072 | 29.513 | 1.00 | 20.23 | A |
| ATOM | 2268 | O | PRO | A | 302 | 20.179 | 60.190 | 30.050 | 1.00 | 19.53 | A |
| ATOM | 2269 | N | PRO | A | 303 | 22.170 | 60.941 | 29.323 | 1.00 | 19.82 | A |
| ATOM | 2270 | CD | PRO | A | 303 | 23.105 | 61.819 | 28.596 | 1.00 | 19.67 | A |
| ATOM | 2271 | CA | PRO | A | 303 | 22.835 | 59.730 | 29.798 | 1.00 | 19.97 | A |
| ATOM | 2272 | CB | PRO | A | 303 | 24.204 | 59.800 | 29.126 | 1.00 | 19.91 | A |
| ATOM | 2273 | CG | PRO | A | 303 | 24.451 | 61.270 | 29.009 | 1.00 | 20.65 | A |
| ATOM | 2274 | C | PRO | A | 303 | 22.919 | 59.817 | 31.318 | 1.00 | 20.45 | A |
| ATOM | 2275 | O | PRO | A | 303 | 22.892 | 60.912 | 31.885 | 1.00 | 19.95 | A |
| ATOM | 2276 | N | ARG | A | 304 | 22.992 | 58.667 | 31.974 | 1.00 | 20.89 | A |
| ATOM | 2277 | CA | ARG | A | 304 | 23.089 | 58.624 | 33.424 | 1.00 | 22.04 | A |
| ATOM | 2278 | CB | ARG | A | 304 | 21.800 | 58.056 | 34.028 | 1.00 | 25.18 | A |
| ATOM | 2279 | CG | ARG | A | 304 | 20.672 | 59.076 | 34.134 | 1.00 | 29.57 | A |
| ATOM | 2280 | CD | ARG | A | 304 | 19.369 | 58.432 | 34.585 | 1.00 | 33.21 | A |
| ATOM | 2281 | NE | ARG | A | 304 | 18.778 | 57.601 | 33.540 | 1.00 | 36.53 | A |
| ATOM | 2282 | CZ | ARG | A | 304 | 18.380 | 58.058 | 32.355 | 1.00 | 37.94 | A |
| ATOM | 2283 | NH1 | ARG | A | 304 | 18.509 | 59.345 | 32.057 | 1.00 | 38.71 | A |
| ATOM | 2284 | NH2 | ARG | A | 304 | 17.851 | 57.229 | 31.466 | 1.00 | 38.86 | A |
| ATOM | 2285 | C | ARG | A | 304 | 24.279 | 57.777 | 33.837 | 1.00 | 20.49 | A |
| ATOM | 2286 | O | ARG | A | 304 | 24.479 | 56.674 | 33.323 | 1.00 | 19.54 | A |
| ATOM | 2287 | N | THR | A | 305 | 25.074 | 58.306 | 34.760 | 1.00 | 19.35 | A |
| ATOM | 2288 | CA | THR | A | 305 | 26.251 | 57.607 | 35.253 | 1.00 | 18.46 | A |
| ATOM | 2289 | CB | THR | A | 305 | 26.901 | 58.378 | 36.418 | 1.00 | 18.86 | A |
| ATOM | 2290 | OG1 | THR | A | 305 | 27.312 | 59.671 | 35.955 | 1.00 | 20.18 | A |
| ATOM | 2291 | CG2 | THR | A | 305 | 28.108 | 57.629 | 36.957 | 1.00 | 19.09 | A |
| ATOM | 2292 | C | THR | A | 305 | 25.864 | 56.216 | 35.729 | 1.00 | 17.72 | A |
| ATOM | 2293 | O | THR | A | 305 | 24.914 | 56.056 | 36.495 | 1.00 | 18.70 | A |
| ATOM | 2294 | N | ILE | A | 306 | 26.598 | 55.210 | 35.268 | 1.00 | 16.09 | A |
| ATOM | 2295 | CA | ILE | A | 306 | 26.306 | 53.837 | 35.654 | 1.00 | 16.08 | A |
| ATOM | 2296 | CB | ILE | A | 306 | 27.031 | 52.829 | 34.738 | 1.00 | 15.93 | A |
| ATOM | 2297 | CG2 | ILE | A | 306 | 26.627 | 51.403 | 35.113 | 1.00 | 14.98 | A |
| ATOM | 2298 | CG1 | ILE | A | 306 | 26.689 | 53.110 | 33.271 | 1.00 | 14.91 | A |
| ATOM | 2299 | CD1 | ILE | A | 306 | 25.204 | 53.036 | 32.943 | 1.00 | 14.74 | A |
| ATOM | 2300 | C | ILE | A | 306 | 26.717 | 53.579 | 37.101 | 1.00 | 17.29 | A |
| ATOM | 2301 | O | ILE | A | 306 | 27.793 | 53.989 | 37.539 | 1.00 | 17.58 | A |
| ATOM | 2302 | N | SER | A | 307 | 25.844 | 52.898 | 37.839 | 1.00 | 18.29 | A |
| ATOM | 2303 | CA | SER | A | 307 | 26.094 | 52.571 | 39.238 | 1.00 | 20.08 | A |
| ATOM | 2304 | CB | SER | A | 307 | 25.339 | 53.539 | 40.151 | 1.00 | 19.58 | A |
| ATOM | 2305 | OG | SER | A | 307 | 23.939 | 53.426 | 39.962 | 1.00 | 20.03 | A |
| ATOM | 2306 | C | SER | A | 307 | 25.605 | 51.154 | 39.496 | 1.00 | 21.11 | A |
| ATOM | 2307 | O | SER | A | 307 | 24.856 | 50.600 | 38.696 | 1.00 | 20.76 | A |
| ATOM | 2308 | N | ASP | A | 308 | 26.029 | 50.569 | 40.612 | 1.00 | 22.96 | A |
| ATOM | 2309 | CA | ASP | A | 308 | 25.604 | 49.219 | 40.966 | 1.00 | 24.81 | A |
| ATOM | 2310 | CB | ASP | A | 308 | 26.243 | 48.800 | 42.292 | 1.00 | 26.28 | A |
| ATOM | 2311 | CG | ASP | A | 308 | 27.729 | 48.532 | 42.165 | 1.00 | 27.47 | A |
| ATOM | 2312 | OD1 | ASP | A | 308 | 28.388 | 49.190 | 41.333 | 1.00 | 28.21 | A |
| ATOM | 2313 | OD2 | ASP | A | 308 | 28.244 | 47.669 | 42.907 | 1.00 | 28.87 | A |
| ATOM | 2314 | C | ASP | A | 308 | 24.085 | 49.210 | 41.099 | 1.00 | 25.44 | A |
| ATOM | 2315 | O | ASP | A | 308 | 23.432 | 48.185 | 40.908 | 1.00 | 24.73 | A |
| ATOM | 2316 | N | GLN | A | 309 | 23.538 | 50.378 | 41.412 | 1.00 | 26.20 | A |
| ATOM | 2317 | CA | GLN | A | 309 | 22.107 | 50.564 | 41.600 | 1.00 | 27.19 | A |
| ATOM | 2318 | CB | GLN | A | 309 | 21.883 | 51.841 | 42.418 | 1.00 | 29.43 | A |
| ATOM | 2319 | CG | GLN | A | 309 | 20.482 | 52.415 | 42.375 | 1.00 | 31.19 | A |
| ATOM | 2320 | CD | GLN | A | 309 | 20.365 | 53.699 | 43.180 | 1.00 | 32.48 | A |
| ATOM | 2321 | OE1 | GLN | A | 309 | 19.472 | 54.514 | 42.948 | 1.00 | 32.65 | A |
| ATOM | 2322 | NE2 | GLN | A | 309 | 21.266 | 53.880 | 44.138 | 1.00 | 32.90 | A |
| ATOM | 2323 | C | GLN | A | 309 | 21.283 | 50.609 | 40.311 | 1.00 | 26.61 | A |
| ATOM | 2324 | O | GLN | A | 309 | 20.108 | 50.240 | 40.314 | 1.00 | 27.21 | A |
| ATOM | 2325 | N | ASN | A | 310 | 21.882 | 51.052 | 39.209 | 1.00 | 24.42 | A |
| ATOM | 2326 | CA | ASN | A | 310 | 21.139 | 51.128 | 37.954 | 1.00 | 22.88 | A |
| ATOM | 2327 | CB | ASN | A | 310 | 21.008 | 52.585 | 37.493 | 1.00 | 22.90 | A |
| ATOM | 2328 | CG | ASN | A | 310 | 22.346 | 53.205 | 37.122 | 1.00 | 22.75 | A |
| ATOM | 2329 | OD1 | ASN | A | 310 | 23.261 | 52.516 | 36.676 | 1.00 | 21.47 | A |
| ATOM | 2330 | ND2 | ASN | A | 310 | 22.455 | 54.519 | 37.287 | 1.00 | 22.83 | A |
| ATOM | 2331 | C | ASN | A | 310 | 21.733 | 50.314 | 36.810 | 1.00 | 21.49 | A |
| ATOM | 2332 | O | ASN | A | 310 | 21.148 | 50.254 | 35.731 | 1.00 | 20.97 | A |
| ATOM | 2333 | N | VAL | A | 311 | 22.881 | 49.687 | 37.042 | 1.00 | 20.33 | A |
| ATOM | 2334 | CA | VAL | A | 311 | 23.546 | 48.921 | 35.991 | 1.00 | 19.01 | A |
| ATOM | 2335 | CB | VAL | A | 311 | 24.889 | 48.332 | 36.490 | 1.00 | 18.91 | A |
| ATOM | 2336 | CG1 | VAL | A | 311 | 24.640 | 47.246 | 37.520 | 1.00 | 18.96 | A |
| ATOM | 2337 | CG2 | VAL | A | 311 | 25.696 | 47.800 | 35.307 | 1.00 | 18.47 | A |
| ATOM | 2338 | C | VAL | A | 311 | 22.694 | 47.804 | 35.389 | 1.00 | 19.05 | A |
| ATOM | 2339 | O | VAL | A | 311 | 22.771 | 47.546 | 34.189 | 1.00 | 17.89 | A |
| ATOM | 2340 | N | ALA | A | 312 | 21.876 | 47.146 | 36.205 | 1.00 | 17.91 | A |
| ATOM | 2341 | CA | ALA | A | 312 | 21.035 | 46.073 | 35.685 | 1.00 | 17.95 | A |
| ATOM | 2342 | CB | ALA | A | 312 | 20.285 | 45.386 | 36.828 | 1.00 | 18.00 | A |
| ATOM | 2343 | C | ALA | A | 312 | 20.046 | 46.609 | 34.652 | 1.00 | 17.45 | A |
| ATOM | 2344 | O | ALA | A | 312 | 19.895 | 46.033 | 33.574 | 1.00 | 17.13 | A |
| ATOM | 2345 | N | ALA | A | 313 | 19.377 | 47.712 | 34.979 | 1.00 | 17.23 | A |
| ATOM | 2346 | CA | ALA | A | 313 | 18.402 | 48.316 | 34.074 | 1.00 | 17.36 | A |
| ATOM | 2347 | CB | ALA | A | 313 | 17.579 | 49.367 | 34.813 | 1.00 | 17.23 | A |
| ATOM | 2348 | C | ALA | A | 313 | 19.075 | 48.946 | 32.858 | 1.00 | 17.38 | A |
| ATOM | 2349 | O | ALA | A | 313 | 18.564 | 48.854 | 31.741 | 1.00 | 17.13 | A |
| ATOM | 2350 | N | ARG | A | 314 | 20.215 | 49.591 | 33.079 | 1.00 | 17.39 | A |
| ATOM | 2351 | CA | ARG | A | 314 | 20.947 | 50.228 | 31.989 | 1.00 | 17.17 | A |
| ATOM | 2352 | CB | ARG | A | 314 | 22.158 | 50.990 | 32.533 | 1.00 | 17.21 | A |
| ATOM | 2353 | CG | ARG | A | 314 | 21.830 | 52.175 | 33.433 | 1.00 | 17.48 | A |
| ATOM | 2354 | CD | ARG | A | 314 | 21.503 | 53.431 | 32.635 | 1.00 | 19.04 | A |
| ATOM | 2355 | NE | ARG | A | 314 | 20.138 | 53.446 | 32.120 | 1.00 | 19.31 | A |
| ATOM | 2356 | CZ | ARG | A | 314 | 19.659 | 54.381 | 31.306 | 1.00 | 19.35 | A |
| ATOM | 2357 | NH1 | ARG | A | 314 | 20.439 | 55.377 | 30.905 | 1.00 | 19.82 | A |
| ATOM | 2358 | NH2 | ARG | A | 314 | 18.396 | 54.338 | 30.908 | 1.00 | 19.86 | A |
| ATOM | 2359 | C | ARG | A | 314 | 21.420 | 49.160 | 31.010 | 1.00 | 17.18 | A |
| ATOM | 2360 | O | ARG | A | 314 | 21.331 | 49.334 | 29.794 | 1.00 | 16.68 | A |
| ATOM | 2361 | N | SER | A | 315 | 21.921 | 48.053 | 31.552 | 1.00 | 16.39 | A |
| ATOM | 2362 | CA | SER | A | 315 | 22.412 | 46.949 | 30.730 | 1.00 | 16.91 | A |
| ATOM | 2363 | CB | SER | A | 315 | 23.072 | 45.879 | 31.605 | 1.00 | 15.94 | A |
| ATOM | 2364 | OG | SER | A | 315 | 24.251 | 46.372 | 32.217 | 1.00 | 16.96 | A |
| ATOM | 2365 | C | SER | A | 315 | 21.284 | 46.319 | 29.929 | 1.00 | 17.74 | A |
| ATOM | 2366 | O | SER | A | 315 | 21.459 | 45.971 | 28.762 | 1.00 | 17.97 | A |
| ATOM | 2367 | N | ASP | A | 316 | 20.123 | 46.170 | 30.556 | 1.00 | 18.36 | A |
| ATOM | 2368 | CA | ASP | A | 316 | 18.985 | 45.573 | 29.875 | 1.00 | 19.21 | A |
| ATOM | 2369 | CB | ASP | A | 316 | 17.793 | 45.468 | 30.828 | 1.00 | 20.93 | A |
| ATOM | 2370 | CG | ASP | A | 316 | 16.731 | 44.517 | 30.321 | 1.00 | 22.66 | A |
| ATOM | 2371 | OD1 | ASP | A | 316 | 17.092 | 43.388 | 29.924 | 1.00 | 24.08 | A |
| ATOM | 2372 | OD2 | ASP | A | 316 | 15.541 | 44.894 | 30.324 | 1.00 | 24.17 | A |
| ATOM | 2373 | C | ASP | A | 316 | 18.611 | 46.398 | 28.645 | 1.00 | 18.91 | A |
| ATOM | 2374 | O | ASP | A | 316 | 18.301 | 45.845 | 27.590 | 1.00 | 18.46 | A |
| ATOM | 2375 | N | LEU | A | 317 | 18.653 | 47.720 | 28.778 | 1.00 | 18.38 | A |
| ATOM | 2376 | CA | LEU | A | 317 | 18.328 | 48.606 | 27.664 | 1.00 | 18.01 | A |
| ATOM | 2377 | CB | LEU | A | 317 | 18.223 | 50.057 | 28.145 | 1.00 | 19.46 | A |
| ATOM | 2378 | CG | LEU | A | 317 | 16.873 | 50.535 | 28.682 | 1.00 | 20.53 | A |
| ATOM | 2379 | CD1 | LEU | A | 317 | 17.023 | 51.923 | 29.288 | 1.00 | 21.52 | A |
| ATOM | 2380 | CD2 | LEU | A | 317 | 15.855 | 50.552 | 27.550 | 1.00 | 20.96 | A |
| ATOM | 2381 | C | LEU | A | 317 | 19.376 | 48.523 | 26.560 | 1.00 | 16.98 | A |
| ATOM | 2382 | O | LEU | A | 317 | 19.041 | 48.400 | 25.382 | 1.00 | 16.42 | A |
| ATOM | 2383 | N | LEU | A | 318 | 20.645 | 48.583 | 26.949 | 1.00 | 15.99 | A |
| ATOM | 2384 | CA | LEU | A | 318 | 21.740 | 48.541 | 25.985 | 1.00 | 15.31 | A |
| ATOM | 2385 | CB | LEU | A | 318 | 23.067 | 48.836 | 26.687 | 1.00 | 14.88 | A |
| ATOM | 2386 | CG | LEU | A | 318 | 24.286 | 48.971 | 25.768 | 1.00 | 15.10 | A |
| ATOM | 2387 | CD1 | LEU | A | 318 | 24.029 | 50.057 | 24.731 | 1.00 | 14.72 | A |
| ATOM | 2388 | CD2 | LEU | A | 318 | 25.514 | 49.305 | 26.600 | 1.00 | 14.28 | A |
| ATOM | 2389 | C | LEU | A | 318 | 21.840 | 47.217 | 25.237 | 1.00 | 15.11 | A |
| ATOM | 2390 | O | LEU | A | 318 | 21.967 | 47.198 | 24.012 | 1.00 | 15.57 | A |
| ATOM | 2391 | N | VAL | A | 319 | 21.786 | 46.108 | 25.967 | 1.00 | 14.42 | A |
| ATOM | 2392 | CA | VAL | A | 319 | 21.871 | 44.799 | 25.331 | 1.00 | 13.85 | A |
| ATOM | 2393 | CB | VAL | A | 319 | 21.846 | 43.664 | 26.380 | 1.00 | 13.47 | A |
| ATOM | 2394 | CG1 | VAL | A | 319 | 21.744 | 42.313 | 25.690 | 1.00 | 13.87 | A |
| ATOM | 2395 | CG2 | VAL | A | 319 | 23.113 | 43.716 | 27.219 | 1.00 | 14.11 | A |
| ATOM | 2396 | C | VAL | A | 319 | 20.719 | 44.611 | 24.346 | 1.00 | 14.16 | A |
| ATOM | 2397 | O | VAL | A | 319 | 20.881 | 43.986 | 23.298 | 1.00 | 13.52 | A |
| ATOM | 2398 | N | ASP | A | 320 | 19.554 | 45.153 | 24.683 | 1.00 | 14.86 | A |
| ATOM | 2399 | CA | ASP | A | 320 | 18.398 | 45.047 | 23.802 | 1.00 | 15.02 | A |
| ATOM | 2400 | CB | ASP | A | 320 | 17.170 | 45.680 | 24.462 | 1.00 | 16.85 | A |
| ATOM | 2401 | CG | ASP | A | 320 | 15.951 | 45.656 | 23.568 | 1.00 | 16.80 | A |
| ATOM | 2402 | OD1 | ASP | A | 320 | 15.498 | 46.742 | 23.155 | 1.00 | 17.63 | A |
| ATOM | 2403 | OD2 | ASP | A | 320 | 15.448 | 44.551 | 23.276 | 1.00 | 18.36 | A |
| ATOM | 2404 | C | ASP | A | 320 | 18.709 | 45.740 | 22.474 | 1.00 | 14.37 | A |
| ATOM | 2405 | O | ASP | A | 320 | 18.373 | 45.231 | 21.404 | 1.00 | 13.82 | A |
| ATOM | 2406 | N | GLN | A | 321 | 19.351 | 46.903 | 22.547 | 1.00 | 13.70 | A |
| ATOM | 2407 | CA | GLN | A | 321 | 19.722 | 47.639 | 21.343 | 1.00 | 12.86 | A |
| ATOM | 2408 | CB | GLN | A | 321 | 20.332 | 48.994 | 21.711 | 1.00 | 11.83 | A |
| ATOM | 2409 | CG | GLN | A | 321 | 19.315 | 50.024 | 22.159 | 1.00 | 12.86 | A |
| ATOM | 2410 | CD | GLN | A | 321 | 18.279 | 50.298 | 21.088 | 1.00 | 14.18 | A |
| ATOM | 2411 | OE1 | GLN | A | 321 | 18.612 | 50.718 | 19.980 | 1.00 | 14.25 | A |
| ATOM | 2412 | NE2 | GLN | A | 321 | 17.013 | 50.059 | 21.412 | 1.00 | 14.45 | A |
| ATOM | 2413 | C | GLN | A | 321 | 20.735 | 46.829 | 20.542 | 1.00 | 12.45 | A |
| ATOM | 2414 | O | GLN | A | 321 | 20.627 | 46.707 | 19.321 | 1.00 | 12.50 | A |
| ATOM | 2415 | N | TRP | A | 322 | 21.722 | 46.277 | 21.240 | 1.00 | 12.86 | A |
| ATOM | 2416 | CA | TRP | A | 322 | 22.749 | 45.475 | 20.590 | 1.00 | 12.89 | A |
| ATOM | 2417 | CB | TRP | A | 322 | 23.773 | 44.969 | 21.609 | 1.00 | 11.98 | A |
| ATOM | 2418 | CG | TRP | A | 322 | 24.703 | 46.009 | 22.163 | 1.00 | 12.42 | A |
| ATOM | 2419 | CD2 | TRP | A | 322 | 25.605 | 45.841 | 23.261 | 1.00 | 11.60 | A |
| ATOM | 2420 | CE2 | TRP | A | 322 | 26.321 | 47.050 | 23.404 | 1.00 | 11.77 | A |
| ATOM | 2421 | CE3 | TRP | A | 322 | 25.879 | 44.782 | 24.138 | 1.00 | 12.07 | A |
| ATOM | 2422 | CD1 | TRP | A | 322 | 24.899 | 47.279 | 21.696 | 1.00 | 11.63 | A |
| ATOM | 2423 | NE1 | TRP | A | 322 | 25.871 | 47.911 | 22.438 | 1.00 | 12.67 | A |
| ATOM | 2424 | CZ2 | TRP | A | 322 | 27.297 | 47.233 | 24.393 | 1.00 | 12.70 | A |
| ATOM | 2425 | CZ3 | TRP | A | 322 | 26.850 | 44.963 | 25.122 | 1.00 | 12.23 | A |
| ATOM | 2426 | CH2 | TRP | A | 322 | 27.545 | 46.180 | 25.240 | 1.00 | 12.89 | A |
| ATOM | 2427 | C | TRP | A | 322 | 22.156 | 44.278 | 19.859 | 1.00 | 13.39 | A |
| ATOM | 2428 | O | TRP | A | 322 | 22.532 | 43.988 | 18.722 | 1.00 | 13.20 | A |
| ATOM | 2429 | N | LYS | A | 323 | 21.236 | 43.575 | 20.511 | 1.00 | 12.94 | A |
| ATOM | 2430 | CA | LYS | A | 323 | 20.632 | 42.404 | 19.890 | 1.00 | 13.54 | A |
| ATOM | 2431 | CB | LYS | A | 323 | 19.855 | 41.593 | 20.930 | 1.00 | 14.32 | A |
| ATOM | 2432 | CG | LYS | A | 323 | 20.799 | 40.871 | 21.888 | 1.00 | 15.76 | A |
| ATOM | 2433 | CD | LYS | A | 323 | 20.079 | 40.005 | 22.904 | 1.00 | 17.29 | A |
| ATOM | 2434 | CE | LYS | A | 323 | 21.086 | 39.198 | 23.708 | 1.00 | 18.79 | A |
| ATOM | 2435 | NZ | LYS | A | 323 | 20.428 | 38.323 | 24.710 | 1.00 | 21.17 | A |
| ATOM | 2436 | C | LYS | A | 323 | 19.762 | 42.766 | 18.700 | 1.00 | 13.15 | A |
| ATOM | 2437 | O | LYS | A | 323 | 19.599 | 41.968 | 17.780 | 1.00 | 13.47 | A |
| ATOM | 2438 | N | LYS | A | 324 | 19.207 | 43.970 | 18.706 | 1.00 | 13.05 | A |
| ATOM | 2439 | CA | LYS | A | 324 | 18.406 | 44.406 | 17.570 | 1.00 | 12.21 | A |
| ATOM | 2440 | CB | LYS | A | 324 | 17.600 | 45.655 | 17.930 | 1.00 | 12.37 | A |
| ATOM | 2441 | CG | LYS | A | 324 | 16.388 | 45.327 | 18.789 | 1.00 | 13.50 | A |
| ATOM | 2442 | CD | LYS | A | 324 | 15.678 | 46.561 | 19.319 | 1.00 | 13.76 | A |
| ATOM | 2443 | CE | LYS | A | 324 | 14.437 | 46.144 | 20.106 | 1.00 | 15.09 | A |
| ATOM | 2444 | NZ | LYS | A | 324 | 13.801 | 47.285 | 20.820 | 1.00 | 16.19 | A |
| ATOM | 2445 | C | LYS | A | 324 | 19.353 | 44.685 | 16.407 | 1.00 | 12.02 | A |
| ATOM | 2446 | O | LYS | A | 324 | 19.080 | 44.304 | 15.270 | 1.00 | 11.97 | A |
| ATOM | 2447 | N | LYS | A | 325 | 20.476 | 45.339 | 16.691 | 1.00 | 11.78 | A |
| ATOM | 2448 | CA | LYS | A | 325 | 21.447 | 45.624 | 15.639 | 1.00 | 11.37 | A |
| ATOM | 2449 | CB | LYS | A | 325 | 22.614 | 46.454 | 16.191 | 1.00 | 10.87 | A |
| ATOM | 2450 | CG | LYS | A | 325 | 23.515 | 47.051 | 15.105 | 1.00 | 10.75 | A |
| ATOM | 2451 | CD | LYS | A | 325 | 24.642 | 47.893 | 15.698 | 1.00 | 11.00 | A |
| ATOM | 2452 | CE | LYS | A | 325 | 25.433 | 48.604 | 14.603 | 1.00 | 11.39 | A |
| ATOM | 2453 | NZ | LYS | A | 325 | 26.585 | 49.360 | 15.164 | 1.00 | 12.31 | A |
| ATOM | 2454 | C | LYS | A | 325 | 21.978 | 44.304 | 15.077 | 1.00 | 11.90 | A |
| ATOM | 2455 | O | LYS | A | 325 | 22.136 | 44.147 | 13.865 | 1.00 | 11.75 | A |
| ATOM | 2456 | N | ALA | A | 326 | 22.240 | 43.351 | 15.968 | 1.00 | 11.87 | A |
| ATOM | 2457 | CA | ALA | A | 326 | 22.760 | 42.047 | 15.567 | 1.00 | 12.64 | A |
| ATOM | 2458 | CB | ALA | A | 326 | 23.024 | 41.192 | 16.804 | 1.00 | 12.66 | A |
| ATOM | 2459 | C | ALA | A | 326 | 21.839 | 41.299 | 14.603 | 1.00 | 13.43 | A |
| ATOM | 2460 | O | ALA | A | 326 | 22.294 | 40.453 | 13.831 | 1.00 | 13.86 | A |
| ATOM | 2461 | N | GLU | A | 327 | 20.545 | 41.599 | 14.653 | 1.00 | 13.80 | A |
| ATOM | 2462 | CA | GLU | A | 327 | 19.584 | 40.942 | 13.770 | 1.00 | 14.81 | A |
| ATOM | 2463 | CB | GLU | A | 327 | 18.153 | 41.327 | 14.152 | 1.00 | 16.43 | A |
| ATOM | 2464 | CG | GLU | A | 327 | 17.575 | 40.504 | 15.277 | 1.00 | 18.89 | A |
| ATOM | 2465 | CD | GLU | A | 327 | 17.577 | 39.020 | 14.960 | 1.00 | 18.94 | A |
| ATOM | 2466 | OE1 | GLU | A | 327 | 16.918 | 38.609 | 13.979 | 1.00 | 20.74 | A |
| ATOM | 2467 | OE2 | GLU | A | 327 | 18.244 | 38.265 | 15.691 | 1.00 | 20.08 | A |
| ATOM | 2468 | C | GLU | A | 327 | 19.810 | 41.296 | 12.308 | 1.00 | 14.28 | A |
| ATOM | 2469 | O | GLU | A | 327 | 19.367 | 40.581 | 11.409 | 1.00 | 15.39 | A |
| ATOM | 2470 | N | LEU | A | 328 | 20.504 | 42.404 | 12.075 | 1.00 | 13.70 | A |
| ATOM | 2471 | CA | LEU | A | 328 | 20.764 | 42.860 | 10.718 | 1.00 | 12.00 | A |
| ATOM | 2472 | CB | LEU | A | 328 | 20.932 | 44.384 | 10.707 | 1.00 | 12.41 | A |
| ATOM | 2473 | CG | LEU | A | 328 | 19.833 | 45.202 | 11.399 | 1.00 | 12.20 | A |
| ATOM | 2474 | CD1 | LEU | A | 328 | 20.108 | 46.687 | 11.208 | 1.00 | 13.82 | A |
| ATOM | 2475 | CD2 | LEU | A | 328 | 18.465 | 44.838 | 10.828 | 1.00 | 13.12 | A |
| ATOM | 2476 | C | LEU | A | 328 | 21.984 | 42.206 | 10.075 | 1.00 | 12.08 | A |
| ATOM | 2477 | O | LEU | A | 328 | 22.240 | 42.411 | 8.888 | 1.00 | 12.28 | A |
| ATOM | 2478 | N | TYR | A | 329 | 22.731 | 41.422 | 10.851 | 1.00 | 11.18 | A |
| ATOM | 2479 | CA | TYR | A | 329 | 23.930 | 40.758 | 10.344 | 1.00 | 11.25 | A |
| ATOM | 2480 | CB | TYR | A | 329 | 25.175 | 41.342 | 11.018 | 1.00 | 11.22 | A |
| ATOM | 2481 | CG | TYR | A | 329 | 25.376 | 42.809 | 10.695 | 1.00 | 11.53 | A |
| ATOM | 2482 | CD1 | TYR | A | 329 | 24.786 | 43.808 | 11.477 | 1.00 | 11.42 | A |
| ATOM | 2483 | CE1 | TYR | A | 329 | 24.917 | 45.157 | 11.147 | 1.00 | 12.31 | A |
| ATOM | 2484 | CD2 | TYR | A | 329 | 26.106 | 43.197 | 9.573 | 1.00 | 11.20 | A |
| ATOM | 2485 | CE2 | TYR | A | 329 | 26.241 | 44.545 | 9.233 | 1.00 | 11.91 | A |
| ATOM | 2486 | CZ | TYR | A | 329 | 25.643 | 45.516 | 10.023 | 1.00 | 11.77 | A |
| ATOM | 2487 | OH | TYR | A | 329 | 25.764 | 46.845 | 9.678 | 1.00 | 12.68 | A |
| ATOM | 2488 | C | TYR | A | 329 | 23.890 | 39.240 | 10.492 | 1.00 | 12.08 | A |
| ATOM | 2489 | O | TYR | A | 329 | 23.029 | 38.703 | 11.188 | 1.00 | 12.83 | A |
| ATOM | 2490 | N | ARG | A | 330 | 24.835 | 38.553 | 9.857 | 1.00 | 12.23 | A |
| ATOM | 2491 | CA | ARG | A | 330 | 24.839 | 37.094 | 9.864 | 1.00 | 12.24 | A |
| ATOM | 2492 | CB | ARG | A | 330 | 25.202 | 36.589 | 8.465 | 1.00 | 11.88 | A |
| ATOM | 2493 | CG | ARG | A | 330 | 24.278 | 37.129 | 7.393 | 1.00 | 11.69 | A |
| ATOM | 2494 | CD | ARG | A | 330 | 24.531 | 36.502 | 6.035 | 1.00 | 12.77 | A |
| ATOM | 2495 | NE | ARG | A | 330 | 23.611 | 37.062 | 5.052 | 1.00 | 12.25 | A |
| ATOM | 2496 | CZ | ARG | A | 330 | 23.371 | 36.544 | 3.852 | 1.00 | 14.24 | A |
| ATOM | 2497 | NH1 | ARG | A | 330 | 23.986 | 35.437 | 3.461 | 1.00 | 15.29 | A |
| ATOM | 2498 | NH2 | ARG | A | 330 | 22.503 | 37.137 | 3.043 | 1.00 | 14.59 | A |
| ATOM | 2499 | C | ARG | A | 330 | 25.649 | 36.311 | 10.894 | 1.00 | 12.88 | A |
| ATOM | 2500 | O | ARG | A | 330 | 25.425 | 35.109 | 11.046 | 1.00 | 14.62 | A |
| ATOM | 2501 | N | THR | A | 331 | 26.584 | 36.950 | 11.588 | 1.00 | 12.94 | A |
| ATOM | 2502 | CA | THR | A | 331 | 27.372 | 36.225 | 12.584 | 1.00 | 12.09 | A |
| ATOM | 2503 | CB | THR | A | 331 | 28.890 | 36.471 | 12.418 | 1.00 | 11.53 | A |
| ATOM | 2504 | OG1 | THR | A | 331 | 29.216 | 37.792 | 12.869 | 1.00 | 11.21 | A |
| ATOM | 2505 | CG2 | THR | A | 331 | 29.298 | 36.310 | 10.956 | 1.00 | 12.94 | A |
| ATOM | 2506 | C | THR | A | 331 | 26.990 | 36.640 | 13.996 | 1.00 | 12.54 | A |
| ATOM | 2507 | O | THR | A | 331 | 26.150 | 37.516 | 14.188 | 1.00 | 13.43 | A |
| ATOM | 2508 | N | ASN | A | 332 | 27.615 | 36.001 | 14.981 | 1.00 | 12.33 | A |
| ATOM | 2509 | CA | ASN | A | 332 | 27.359 | 36.315 | 16.382 | 1.00 | 12.94 | A |
| ATOM | 2510 | CB | ASN | A | 332 | 27.323 | 35.028 | 17.218 | 1.00 | 13.86 | A |
| ATOM | 2511 | CG | ASN | A | 332 | 28.676 | 34.344 | 17.310 | 1.00 | 15.72 | A |
| ATOM | 2512 | OD1 | ASN | A | 332 | 29.431 | 34.298 | 16.342 | 1.00 | 17.44 | A |
| ATOM | 2513 | ND2 | ASN | A | 332 | 28.979 | 33.789 | 18.481 | 1.00 | 17.45 | A |
| ATOM | 2514 | C | ASN | A | 332 | 28.433 | 37.264 | 16.907 | 1.00 | 12.83 | A |
| ATOM | 2515 | O | ASN | A | 332 | 28.721 | 37.300 | 18.105 | 1.00 | 13.18 | A |
| ATOM | 2516 | N | VAL | A | 333 | 29.024 | 38.027 | 15.990 | 1.00 | 11.52 | A |
| ATOM | 2517 | CA | VAL | A | 333 | 30.055 | 39.005 | 16.323 | 1.00 | 11.89 | A |
| ATOM | 2518 | CB | VAL | A | 333 | 31.356 | 38.731 | 15.536 | 1.00 | 11.95 | A |
| ATOM | 2519 | CG1 | VAL | A | 333 | 32.431 | 39.733 | 15.937 | 1.00 | 12.37 | A |
| ATOM | 2520 | CG2 | VAL | A | 333 | 31.827 | 37.312 | 15.796 | 1.00 | 13.46 | A |
| ATOM | 2521 | C | VAL | A | 333 | 29.491 | 40.363 | 15.918 | 1.00 | 11.33 | A |
| ATOM | 2522 | O | VAL | A | 333 | 29.179 | 40.582 | 14.752 | 1.00 | 11.56 | A |
| ATOM | 2523 | N | LEU | A | 334 | 29.368 | 41.273 | 16.880 | 1.00 | 10.67 | A |
| ATOM | 2524 | CA | LEU | A | 334 | 28.789 | 42.584 | 16.618 | 1.00 | 10.61 | A |
| ATOM | 2525 | CB | LEU | A | 334 | 27.590 | 42.796 | 17.550 | 1.00 | 10.83 | A |
| ATOM | 2526 | CG | LEU | A | 334 | 26.775 | 44.076 | 17.371 | 1.00 | 11.05 | A |
| ATOM | 2527 | CD1 | LEU | A | 334 | 26.045 | 44.025 | 16.036 | 1.00 | 10.50 | A |
| ATOM | 2528 | CD2 | LEU | A | 334 | 25.780 | 44.215 | 18.514 | 1.00 | 11.60 | A |
| ATOM | 2529 | C | LEU | A | 334 | 29.759 | 43.750 | 16.770 | 1.00 | 10.02 | A |
| ATOM | 2530 | O | LEU | A | 334 | 30.512 | 43.827 | 17.739 | 1.00 | 10.77 | A |
| ATOM | 2531 | N | LEU | A | 335 | 29.724 | 44.663 | 15.805 | 1.00 | 10.11 | A |
| ATOM | 2532 | CA | LEU | A | 335 | 30.585 | 45.839 | 15.827 | 1.00 | 9.42 | A |
| ATOM | 2533 | CB | LEU | A | 335 | 31.087 | 46.154 | 14.418 | 1.00 | 8.92 | A |
| ATOM | 2534 | CG | LEU | A | 335 | 31.857 | 47.471 | 14.284 | 1.00 | 9.26 | A |
| ATOM | 2535 | CD1 | LEU | A | 335 | 33.158 | 47.388 | 15.065 | 1.00 | 11.05 | A |
| ATOM | 2536 | CD2 | LEU | A | 335 | 32.128 | 47.755 | 12.813 | 1.00 | 11.26 | A |
| ATOM | 2537 | C | LEU | A | 335 | 29.812 | 47.041 | 16.351 | 1.00 | 9.28 | A |
| ATOM | 2538 | O | LEU | A | 335 | 28.774 | 47.405 | 15.800 | 1.00 | 10.12 | A |
| ATOM | 2539 | N | ILE | A | 336 | 30.324 | 47.654 | 17.412 | 1.00 | 9.16 | A |
| ATOM | 2540 | CA | ILE | A | 336 | 29.686 | 48.824 | 17.996 | 1.00 | 9.09 | A |
| ATOM | 2541 | CB | ILE | A | 336 | 29.152 | 48.535 | 19.424 | 1.00 | 9.68 | A |
| ATOM | 2542 | CG2 | ILE | A | 336 | 28.562 | 49.805 | 20.024 | 1.00 | 10.04 | A |
| ATOM | 2543 | CG1 | ILE | A | 336 | 28.089 | 47.432 | 19.383 | 1.00 | 9.33 | A |
| ATOM | 2544 | CD1 | ILE | A | 336 | 26.848 | 47.786 | 18.595 | 1.00 | 10.48 | A |
| ATOM | 2545 | C | ILE | A | 336 | 30.674 | 49.988 | 18.074 | 1.00 | 8.75 | A |
| ATOM | 2546 | O | ILE | A | 336 | 31.454 | 50.087 | 19.018 | 1.00 | 9.03 | A |
| ATOM | 2547 | N | PRO | A | 337 | 30.674 | 50.871 | 17.061 | 1.00 | 8.98 | A |
| ATOM | 2548 | CD | PRO | A | 337 | 29.982 | 50.807 | 15.763 | 1.00 | 8.96 | A |
| ATOM | 2549 | CA | PRO | A | 337 | 31.598 | 52.008 | 17.104 | 1.00 | 9.22 | A |
| ATOM | 2550 | CB | PRO | A | 337 | 31.359 | 52.702 | 15.764 | 1.00 | 8.51 | A |
| ATOM | 2551 | CG | PRO | A | 337 | 30.921 | 51.585 | 14.871 | 1.00 | 8.89 | A |
| ATOM | 2552 | C | PRO | A | 337 | 31.217 | 52.913 | 18.271 | 1.00 | 8.95 | A |
| ATOM | 2553 | O | PRO | A | 337 | 30.039 | 53.021 | 18.609 | 1.00 | 10.28 | A |
| ATOM | 2554 | N | LEU | A | 338 | 32.210 | 53.548 | 18.886 | 1.00 | 8.46 | A |
| ATOM | 2555 | CA | LEU | A | 338 | 31.956 | 54.462 | 19.994 | 1.00 | 8.68 | A |
| ATOM | 2556 | CB | LEU | A | 338 | 32.335 | 53.815 | 21.329 | 1.00 | 9.38 | A |
| ATOM | 2557 | CG | LEU | A | 338 | 31.988 | 54.674 | 22.549 | 1.00 | 10.48 | A |
| ATOM | 2558 | CD1 | LEU | A | 338 | 30.481 | 54.631 | 22.801 | 1.00 | 11.37 | A |
| ATOM | 2559 | CD2 | LEU | A | 338 | 32.732 | 54.160 | 23.768 | 1.00 | 11.85 | A |
| ATOM | 2560 | C | LEU | A | 338 | 32.781 | 55.727 | 19.775 | 1.00 | 9.16 | A |
| ATOM | 2561 | O | LEU | A | 338 | 33.925 | 55.823 | 20.220 | 1.00 | 9.47 | A |
| ATOM | 2562 | N | GLY | A | 339 | 32.194 | 56.695 | 19.079 | 1.00 | 9.45 | A |
| ATOM | 2563 | CA | GLY | A | 339 | 32.914 | 57.926 | 18.807 | 1.00 | 9.23 | A |
| ATOM | 2564 | C | GLY | A | 339 | 32.105 | 58.931 | 18.017 | 1.00 | 9.65 | A |
| ATOM | 2565 | O | GLY | A | 339 | 30.938 | 58.694 | 17.703 | 1.00 | 9.88 | A |
| ATOM | 2566 | N | ASP | A | 340 | 32.739 | 60.054 | 17.690 | 1.00 | 9.87 | A |
| ATOM | 2567 | CA | ASP | A | 340 | 32.103 | 61.130 | 16.943 | 1.00 | 9.83 | A |
| ATOM | 2568 | CB | ASP | A | 340 | 31.101 | 61.863 | 17.841 | 1.00 | 10.38 | A |
| ATOM | 2569 | CG | ASP | A | 340 | 30.001 | 62.568 | 17.062 | 1.00 | 11.64 | A |
| ATOM | 2570 | OD1 | ASP | A | 340 | 30.195 | 62.883 | 15.865 | 1.00 | 11.74 | A |
| ATOM | 2571 | OD2 | ASP | A | 340 | 28.937 | 62.826 | 17.667 | 1.00 | 12.92 | A |
| ATOM | 2572 | C | ASP | A | 340 | 33.214 | 62.090 | 16.518 | 1.00 | 10.19 | A |
| ATOM | 2573 | O | ASP | A | 340 | 34.401 | 61.792 | 16.683 | 1.00 | 9.86 | A |
| ATOM | 2574 | N | ASP | A | 341 | 32.825 | 63.244 | 15.991 | 1.00 | 10.61 | A |
| ATOM | 2575 | CA | ASP | A | 341 | 33.785 | 64.242 | 15.532 | 1.00 | 10.48 | A |
| ATOM | 2576 | CB | ASP | A | 341 | 33.048 | 65.413 | 14.884 | 1.00 | 12.00 | A |
| ATOM | 2577 | CG | ASP | A | 341 | 32.468 | 65.055 | 13.534 | 1.00 | 13.27 | A |
| ATOM | 2578 | OD1 | ASP | A | 341 | 32.494 | 63.861 | 13.173 | 1.00 | 13.14 | A |
| ATOM | 2579 | OD2 | ASP | A | 341 | 31.984 | 65.972 | 12.832 | 1.00 | 17.12 | A |
| ATOM | 2580 | C | ASP | A | 341 | 34.694 | 64.765 | 16.637 | 1.00 | 10.31 | A |
| ATOM | 2581 | O | ASP | A | 341 | 34.221 | 65.254 | 17.664 | 1.00 | 11.01 | A |
| ATOM | 2582 | N | PHE | A | 342 | 36.000 | 64.657 | 16.404 | 1.00 | 9.63 | A |
| ATOM | 2583 | CA | PHE | A | 342 | 37.018 | 65.114 | 17.342 | 1.00 | 9.96 | A |
| ATOM | 2584 | CB | PHE | A | 342 | 37.207 | 66.632 | 17.204 | 1.00 | 10.12 | A |
| ATOM | 2585 | CG | PHE | A | 342 | 37.737 | 67.057 | 15.856 | 1.00 | 10.10 | A |
| ATOM | 2586 | CD1 | PHE | A | 342 | 36.873 | 67.462 | 14.838 | 1.00 | 10.88 | A |
| ATOM | 2587 | CD2 | PHE | A | 342 | 39.106 | 67.025 | 15.598 | 1.00 | 9.64 | A |
| ATOM | 2588 | CE1 | PHE | A | 342 | 37.367 | 67.828 | 13.583 | 1.00 | 10.53 | A |
| ATOM | 2589 | CE2 | PHE | A | 342 | 39.608 | 67.387 | 14.354 | 1.00 | 9.71 | A |
| ATOM | 2590 | CZ | PHE | A | 342 | 38.737 | 67.790 | 13.342 | 1.00 | 9.85 | A |
| ATOM | 2591 | C | PHE | A | 342 | 36.746 | 64.731 | 18.800 | 1.00 | 10.16 | A |
| ATOM | 2592 | O | PHE | A | 342 | 36.911 | 65.543 | 19.721 | 1.00 | 11.34 | A |
| ATOM | 2593 | N | ARG | A | 343 | 36.339 | 63.482 | 19.002 | 1.00 | 9.86 | A |
| ATOM | 2594 | CA | ARG | A | 343 | 36.068 | 62.981 | 20.345 | 1.00 | 10.14 | A |
| ATOM | 2595 | CB | ARG | A | 343 | 35.056 | 61.830 | 20.306 | 1.00 | 9.08 | A |
| ATOM | 2596 | CG | ARG | A | 343 | 33.613 | 62.266 | 20.104 | 1.00 | 9.69 | A |
| ATOM | 2597 | CD | ARG | A | 343 | 33.191 | 63.269 | 21.172 | 1.00 | 10.14 | A |
| ATOM | 2598 | NE | ARG | A | 343 | 31.763 | 63.572 | 21.122 | 1.00 | 11.19 | A |
| ATOM | 2599 | CZ | ARG | A | 343 | 30.818 | 62.853 | 21.722 | 1.00 | 10.93 | A |
| ATOM | 2600 | NH1 | ARG | A | 343 | 31.135 | 61.774 | 22.427 | 1.00 | 10.27 | A |
| ATOM | 2601 | NH2 | ARG | A | 343 | 29.550 | 63.226 | 21.630 | 1.00 | 10.56 | A |
| ATOM | 2602 | C | ARG | A | 343 | 37.340 | 62.510 | 21.041 | 1.00 | 11.10 | A |
| ATOM | 2603 | O | ARG | A | 343 | 38.410 | 62.413 | 20.427 | 1.00 | 10.83 | A |
| ATOM | 2604 | N | PHE | A | 344 | 37.203 | 62.220 | 22.331 | 1.00 | 12.00 | A |
| ATOM | 2605 | CA | PHE | A | 344 | 38.306 | 61.762 | 23.168 | 1.00 | 12.90 | A |
| ATOM | 2606 | CB | PHE | A | 344 | 38.806 | 60.398 | 22.681 | 1.00 | 12.98 | A |
| ATOM | 2607 | CG | PHE | A | 344 | 37.794 | 59.305 | 22.851 | 1.00 | 12.50 | A |
| ATOM | 2608 | CD1 | PHE | A | 344 | 36.975 | 58.922 | 21.796 | 1.00 | 12.67 | A |
| ATOM | 2609 | CD2 | PHE | A | 344 | 37.608 | 58.710 | 24.095 | 1.00 | 13.77 | A |
| ATOM | 2610 | CE1 | PHE | A | 344 | 35.981 | 57.966 | 21.975 | 1.00 | 12.90 | A |
| ATOM | 2611 | CE2 | PHE | A | 344 | 36.617 | 57.752 | 24.286 | 1.00 | 13.51 | A |
| ATOM | 2612 | CZ | PHE | A | 344 | 35.801 | 57.379 | 23.224 | 1.00 | 12.69 | A |
| ATOM | 2613 | C | PHE | A | 344 | 39.432 | 62.784 | 23.222 | 1.00 | 14.41 | A |
| ATOM | 2614 | O | PHE | A | 344 | 40.609 | 62.458 | 23.059 | 1.00 | 14.24 | A |
| ATOM | 2615 | N | LYS | A | 345 | 39.040 | 64.027 | 23.486 | 1.00 | 15.81 | A |
| ATOM | 2616 | CA | LYS | A | 345 | 39.960 | 65.152 | 23.566 | 1.00 | 18.16 | A |
| ATOM | 2617 | CB | LYS | A | 345 | 39.298 | 66.388 | 22.959 | 1.00 | 19.39 | A |
| ATOM | 2618 | CG | LYS | A | 345 | 40.104 | 67.660 | 23.093 | 1.00 | 21.38 | A |
| ATOM | 2619 | CD | LYS | A | 345 | 39.333 | 68.841 | 22.535 | 1.00 | 23.37 | A |
| ATOM | 2620 | CE | LYS | A | 345 | 40.093 | 70.139 | 22.721 | 1.00 | 24.23 | A |
| ATOM | 2621 | NZ | LYS | A | 345 | 39.320 | 71.293 | 22.183 | 1.00 | 24.71 | A |
| ATOM | 2622 | C | LYS | A | 345 | 40.388 | 65.463 | 24.997 | 1.00 | 19.45 | A |
| ATOM | 2623 | O | LYS | A | 345 | 41.571 | 65.661 | 25.268 | 1.00 | 21.28 | A |
| ATOM | 2624 | N | GLN | A | 346 | 39.419 | 65.514 | 25.903 | 1.00 | 20.22 | A |
| ATOM | 2625 | CA | GLN | A | 346 | 39.691 | 65.820 | 27.303 | 1.00 | 20.84 | A |
| ATOM | 2626 | CB | GLN | A | 346 | 38.539 | 66.640 | 27.879 | 1.00 | 23.49 | A |
| ATOM | 2627 | CG | GLN | A | 346 | 38.262 | 67.921 | 27.116 | 1.00 | 27.90 | A |
| ATOM | 2628 | CD | GLN | A | 346 | 36.943 | 68.547 | 27.504 | 1.00 | 30.08 | A |
| ATOM | 2629 | OE1 | GLN | A | 346 | 36.708 | 68.850 | 28.673 | 1.00 | 32.64 | A |
| ATOM | 2630 | NE2 | GLN | A | 346 | 36.068 | 68.742 | 26.522 | 1.00 | 31.15 | A |
| ATOM | 2631 | C | GLN | A | 346 | 39.882 | 64.562 | 28.142 | 1.00 | 20.09 | A |
| ATOM | 2632 | O | GLN | A | 346 | 39.241 | 63.540 | 27.902 | 1.00 | 18.93 | A |
| ATOM | 2633 | N | ASN | A | 347 | 40.763 | 64.647 | 29.135 | 1.00 | 19.30 | A |
| ATOM | 2634 | CA | ASN | A | 347 | 41.021 | 63.515 | 30.015 | 1.00 | 18.58 | A |
| ATOM | 2635 | CB | ASN | A | 347 | 42.034 | 63.896 | 31.098 | 1.00 | 20.07 | A |
| ATOM | 2636 | CG | ASN | A | 347 | 43.427 | 64.081 | 30.546 | 1.00 | 21.88 | A |
| ATOM | 2637 | OD1 | ASN | A | 347 | 43.973 | 63.181 | 29.909 | 1.00 | 22.61 | A |
| ATOM | 2638 | ND2 | ASN | A | 347 | 44.014 | 65.250 | 30.787 | 1.00 | 23.12 | A |
| ATOM | 2639 | C | ASN | A | 347 | 39.725 | 63.067 | 30.669 | 1.00 | 17.30 | A |
| ATOM | 2640 | O | ASN | A | 347 | 39.459 | 61.873 | 30.780 | 1.00 | 16.74 | A |
| ATOM | 2641 | N | THR | A | 348 | 38.919 | 64.036 | 31.094 | 1.00 | 16.16 | A |
| ATOM | 2642 | CA | THR | A | 348 | 37.647 | 63.738 | 31.737 | 1.00 | 15.64 | A |
| ATOM | 2643 | CB | THR | A | 348 | 36.932 | 65.032 | 32.191 | 1.00 | 16.49 | A |
| ATOM | 2644 | OG1 | THR | A | 348 | 36.836 | 65.945 | 31.090 | 1.00 | 17.92 | A |
| ATOM | 2645 | CG2 | THR | A | 348 | 37.702 | 65.692 | 33.332 | 1.00 | 17.02 | A |
| ATOM | 2646 | C | THR | A | 348 | 36.732 | 62.956 | 30.803 | 1.00 | 14.77 | A |
| ATOM | 2647 | O | THR | A | 348 | 35.929 | 62.139 | 31.256 | 1.00 | 14.29 | A |
| ATOM | 2648 | N | GLU | A | 349 | 36.851 | 63.207 | 29.501 | 1.00 | 12.93 | A |
| ATOM | 2649 | CA | GLU | A | 349 | 36.033 | 62.497 | 28.523 | 1.00 | 12.93 | A |
| ATOM | 2650 | CB | GLU | A | 349 | 36.149 | 63.146 | 27.142 | 1.00 | 12.36 | A |
| ATOM | 2651 | CG | GLU | A | 349 | 35.352 | 62.407 | 26.079 | 1.00 | 12.45 | A |
| ATOM | 2652 | CD | GLU | A | 349 | 35.495 | 62.998 | 24.691 | 1.00 | 12.49 | A |
| ATOM | 2653 | OE1 | GLU | A | 349 | 34.930 | 62.400 | 23.754 | 1.00 | 13.64 | A |
| ATOM | 2654 | OE2 | GLU | A | 349 | 36.158 | 64.044 | 24.535 | 1.00 | 13.87 | A |
| ATOM | 2655 | C | GLU | A | 349 | 36.469 | 61.037 | 28.437 | 1.00 | 12.59 | A |
| ATOM | 2656 | O | GLU | A | 349 | 35.632 | 60.134 | 28.384 | 1.00 | 12.35 | A |
| ATOM | 2657 | N | TRP | A | 350 | 37.778 | 60.804 | 28.414 | 1.00 | 12.37 | A |
| ATOM | 2658 | CA | TRP | A | 350 | 38.283 | 59.438 | 28.361 | 1.00 | 11.74 | A |
| ATOM | 2659 | CB | TRP | A | 350 | 39.816 | 59.412 | 28.372 | 1.00 | 10.85 | A |
| ATOM | 2660 | CG | TRP | A | 350 | 40.438 | 59.655 | 27.030 | 1.00 | 10.74 | A |
| ATOM | 2661 | CD2 | TRP | A | 350 | 40.744 | 58.664 | 26.042 | 1.00 | 11.29 | A |
| ATOM | 2662 | CE2 | TRP | A | 350 | 41.266 | 59.341 | 24.919 | 1.00 | 11.06 | A |
| ATOM | 2663 | CE3 | TRP | A | 350 | 40.625 | 57.269 | 25.997 | 1.00 | 12.34 | A |
| ATOM | 2664 | CD1 | TRP | A | 350 | 40.780 | 60.861 | 26.482 | 1.00 | 11.42 | A |
| ATOM | 2665 | NE1 | TRP | A | 350 | 41.279 | 60.679 | 25.215 | 1.00 | 11.21 | A |
| ATOM | 2666 | CZ2 | TRP | A | 350 | 41.668 | 58.669 | 23.759 | 1.00 | 11.09 | A |
| ATOM | 2667 | CZ3 | TRP | A | 350 | 41.025 | 56.601 | 24.844 | 1.00 | 12.75 | A |
| ATOM | 2668 | CH2 | TRP | A | 350 | 41.539 | 57.303 | 23.740 | 1.00 | 12.45 | A |
| ATOM | 2669 | C | TRP | A | 350 | 37.752 | 58.675 | 29.569 | 1.00 | 12.68 | A |
| ATOM | 2670 | O | TRP | A | 350 | 37.269 | 57.552 | 29.443 | 1.00 | 12.87 | A |
| ATOM | 2671 | N | ASP | A | 351 | 37.828 | 59.300 | 30.739 | 1.00 | 13.49 | A |
| ATOM | 2672 | CA | ASP | A | 351 | 37.357 | 58.670 | 31.965 | 1.00 | 14.43 | A |
| ATOM | 2673 | CB | ASP | A | 351 | 37.669 | 59.542 | 33.184 | 1.00 | 15.49 | A |
| ATOM | 2674 | CG | ASP | A | 351 | 39.148 | 59.628 | 33.482 | 1.00 | 18.09 | A |
| ATOM | 2675 | OD1 | ASP | A | 351 | 39.866 | 58.633 | 33.250 | 1.00 | 19.99 | A |
| ATOM | 2676 | OD2 | ASP | A | 351 | 39.588 | 60.690 | 33.970 | 1.00 | 20.85 | A |
| ATOM | 2677 | C | ASP | A | 351 | 35.864 | 58.374 | 31.960 | 1.00 | 13.60 | A |
| ATOM | 2678 | O | ASP | A | 351 | 35.451 | 57.260 | 32.286 | 1.00 | 14.13 | A |
| ATOM | 2679 | N | VAL | A | 352 | 35.053 | 59.362 | 31.598 | 1.00 | 13.66 | A |
| ATOM | 2680 | CA | VAL | A | 352 | 33.612 | 59.165 | 31.609 | 1.00 | 13.97 | A |
| ATOM | 2681 | CB | VAL | A | 352 | 32.855 | 60.483 | 31.278 | 1.00 | 15.02 | A |
| ATOM | 2682 | CG1 | VAL | A | 352 | 32.894 | 60.769 | 29.791 | 1.00 | 15.28 | A |
| ATOM | 2683 | CG2 | VAL | A | 352 | 31.429 | 60.401 | 31.788 | 1.00 | 16.97 | A |
| ATOM | 2684 | C | VAL | A | 352 | 33.160 | 58.043 | 30.675 | 1.00 | 14.02 | A |
| ATOM | 2685 | O | VAL | A | 352 | 32.286 | 57.256 | 31.031 | 1.00 | 14.06 | A |
| ATOM | 2686 | N | GLN | A | 353 | 33.757 | 57.947 | 29.491 | 1.00 | 12.99 | A |
| ATOM | 2687 | CA | GLN | A | 353 | 33.367 | 56.888 | 28.569 | 1.00 | 12.34 | A |
| ATOM | 2688 | CB | GLN | A | 353 | 33.887 | 57.187 | 27.154 | 1.00 | 11.41 | A |
| ATOM | 2689 | CG | GLN | A | 353 | 33.348 | 58.484 | 26.546 | 1.00 | 11.76 | A |
| ATOM | 2690 | CD | GLN | A | 353 | 31.900 | 58.381 | 26.080 | 1.00 | 11.91 | A |
| ATOM | 2691 | OE1 | GLN | A | 353 | 31.080 | 57.703 | 26.698 | 1.00 | 12.30 | A |
| ATOM | 2692 | NE2 | GLN | A | 353 | 31.578 | 59.072 | 24.991 | 1.00 | 11.88 | A |
| ATOM | 2693 | C | GLN | A | 353 | 33.890 | 55.528 | 29.042 | 1.00 | 12.28 | A |
| ATOM | 2694 | O | GLN | A | 353 | 33.130 | 54.561 | 29.132 | 1.00 | 12.59 | A |
| ATOM | 2695 | N | ARG | A | 354 | 35.179 | 55.455 | 29.365 | 1.00 | 11.64 | A |
| ATOM | 2696 | CA | ARG | A | 354 | 35.775 | 54.192 | 29.799 | 1.00 | 12.41 | A |
| ATOM | 2697 | CB | ARG | A | 354 | 37.286 | 54.340 | 29.993 | 1.00 | 12.29 | A |
| ATOM | 2698 | CG | ARG | A | 354 | 37.955 | 53.065 | 30.508 | 1.00 | 12.77 | A |
| ATOM | 2699 | CD | ARG | A | 354 | 39.456 | 53.247 | 30.716 | 1.00 | 14.15 | A |
| ATOM | 2700 | NE | ARG | A | 354 | 39.768 | 54.280 | 31.702 | 1.00 | 15.21 | A |
| ATOM | 2701 | CZ | ARG | A | 354 | 39.593 | 54.156 | 33.016 | 1.00 | 15.91 | A |
| ATOM | 2702 | NH1 | ARG | A | 354 | 39.104 | 53.032 | 33.526 | 1.00 | 16.33 | A |
| ATOM | 2703 | NH2 | ARG | A | 354 | 39.912 | 55.158 | 33.824 | 1.00 | 16.46 | A |
| ATOM | 2704 | C | ARG | A | 354 | 35.183 | 53.595 | 31.069 | 1.00 | 12.83 | A |
| ATOM | 2705 | O | ARG | A | 354 | 34.817 | 52.422 | 31.089 | 1.00 | 12.74 | A |
| ATOM | 2706 | N | VAL | A | 355 | 35.102 | 54.395 | 32.128 | 1.00 | 12.88 | A |
| ATOM | 2707 | CA | VAL | A | 355 | 34.577 | 53.912 | 33.401 | 1.00 | 13.28 | A |
| ATOM | 2708 | CB | VAL | A | 355 | 34.650 | 55.009 | 34.485 | 1.00 | 13.60 | A |
| ATOM | 2709 | CG1 | VAL | A | 355 | 34.053 | 54.498 | 35.789 | 1.00 | 16.28 | A |
| ATOM | 2710 | CG2 | VAL | A | 355 | 36.100 | 55.418 | 34.706 | 1.00 | 14.36 | A |
| ATOM | 2711 | C | VAL | A | 355 | 33.146 | 53.399 | 33.313 | 1.00 | 12.98 | A |
| ATOM | 2712 | O | VAL | A | 355 | 32.836 | 52.322 | 33.826 | 1.00 | 13.34 | A |
| ATOM | 2713 | N | ASN | A | 356 | 32.270 | 54.162 | 32.670 | 1.00 | 13.08 | A |
| ATOM | 2714 | CA | ASN | A | 356 | 30.886 | 53.740 | 32.550 | 1.00 | 12.48 | A |
| ATOM | 2715 | CB | ASN | A | 356 | 30.035 | 54.877 | 31.990 | 1.00 | 12.67 | A |
| ATOM | 2716 | CG | ASN | A | 356 | 29.735 | 55.928 | 33.036 | 1.00 | 13.47 | A |
| ATOM | 2717 | OD1 | ASN | A | 356 | 29.056 | 55.647 | 34.029 | 1.00 | 13.87 | A |
| ATOM | 2718 | ND2 | ASN | A | 356 | 30.250 | 57.138 | 32.835 | 1.00 | 13.69 | A |
| ATOM | 2719 | C | ASN | A | 356 | 30.751 | 52.472 | 31.716 | 1.00 | 12.85 | A |
| ATOM | 2720 | O | ASN | A | 356 | 29.980 | 51.576 | 32.066 | 1.00 | 12.64 | A |
| ATOM | 2721 | N | TYR | A | 357 | 31.504 | 52.370 | 30.627 | 1.00 | 12.12 | A |
| ATOM | 2722 | CA | TYR | A | 357 | 31.415 | 51.162 | 29.823 | 1.00 | 12.64 | A |
| ATOM | 2723 | CB | TYR | A | 357 | 32.072 | 51.358 | 28.451 | 1.00 | 12.21 | A |
| ATOM | 2724 | CG | TYR | A | 357 | 31.083 | 51.882 | 27.439 | 1.00 | 11.70 | A |
| ATOM | 2725 | CD1 | TYR | A | 357 | 30.894 | 53.251 | 27.261 | 1.00 | 11.24 | A |
| ATOM | 2726 | CE1 | TYR | A | 357 | 29.917 | 53.736 | 26.393 | 1.00 | 11.52 | A |
| ATOM | 2727 | CD2 | TYR | A | 357 | 30.273 | 51.004 | 26.718 | 1.00 | 12.23 | A |
| ATOM | 2728 | CE2 | TYR | A | 357 | 29.291 | 51.477 | 25.850 | 1.00 | 12.04 | A |
| ATOM | 2729 | CZ | TYR | A | 357 | 29.118 | 52.842 | 25.693 | 1.00 | 11.95 | A |
| ATOM | 2730 | OH | TYR | A | 357 | 28.142 | 53.306 | 24.847 | 1.00 | 12.56 | A |
| ATOM | 2731 | C | TYR | A | 357 | 32.015 | 49.970 | 30.558 | 1.00 | 12.74 | A |
| ATOM | 2732 | O | TYR | A | 357 | 31.524 | 48.848 | 30.430 | 1.00 | 12.97 | A |
| ATOM | 2733 | N | GLU | A | 358 | 33.064 | 50.206 | 31.341 | 1.00 | 13.29 | A |
| ATOM | 2734 | CA | GLU | A | 358 | 33.669 | 49.118 | 32.104 | 1.00 | 13.40 | A |
| ATOM | 2735 | CB | GLU | A | 358 | 34.871 | 49.612 | 32.910 | 1.00 | 15.05 | A |
| ATOM | 2736 | CG | GLU | A | 358 | 36.138 | 49.838 | 32.092 | 1.00 | 16.80 | A |
| ATOM | 2737 | CD | GLU | A | 358 | 37.310 | 50.270 | 32.956 | 1.00 | 18.32 | A |
| ATOM | 2738 | OE1 | GLU | A | 358 | 37.115 | 50.453 | 34.176 | 1.00 | 20.90 | A |
| ATOM | 2739 | OE2 | GLU | A | 358 | 38.426 | 50.430 | 32.421 | 1.00 | 18.81 | A |
| ATOM | 2740 | C | GLU | A | 358 | 32.631 | 48.526 | 33.059 | 1.00 | 13.69 | A |
| ATOM | 2741 | O | GLU | A | 358 | 32.554 | 47.312 | 33.221 | 1.00 | 13.52 | A |
| ATOM | 2742 | N | ARG | A | 359 | 31.833 | 49.387 | 33.686 | 1.00 | 13.66 | A |
| ATOM | 2743 | CA | ARG | A | 359 | 30.804 | 48.920 | 34.616 | 1.00 | 14.36 | A |
| ATOM | 2744 | CB | ARG | A | 359 | 30.150 | 50.103 | 35.327 | 1.00 | 14.85 | A |
| ATOM | 2745 | CG | ARG | A | 359 | 31.061 | 50.815 | 36.303 | 1.00 | 17.92 | A |
| ATOM | 2746 | CD | ARG | A | 359 | 30.433 | 52.125 | 36.740 | 1.00 | 20.12 | A |
| ATOM | 2747 | NE | ARG | A | 359 | 31.315 | 52.896 | 37.608 | 1.00 | 22.12 | A |
| ATOM | 2748 | CZ | ARG | A | 359 | 31.333 | 54.224 | 37.653 | 1.00 | 23.24 | A |
| ATOM | 2749 | NH1 | ARG | A | 359 | 30.516 | 54.926 | 36.874 | 1.00 | 23.08 | A |
| ATOM | 2750 | NH2 | ARG | A | 359 | 32.168 | 54.850 | 38.473 | 1.00 | 24.71 | A |
| ATOM | 2751 | C | ARG | A | 359 | 29.737 | 48.108 | 33.893 | 1.00 | 13.89 | A |
| ATOM | 2752 | O | ARG | A | 359 | 29.218 | 47.130 | 34.432 | 1.00 | 14.23 | A |
| ATOM | 2753 | N | LEU | A | 360 | 29.406 | 48.517 | 32.672 | 1.00 | 13.60 | A |
| ATOM | 2754 | CA | LEU | A | 360 | 28.410 | 47.803 | 31.886 | 1.00 | 13.35 | A |
| ATOM | 2755 | CB | LEU | A | 360 | 28.031 | 48.621 | 30.645 | 1.00 | 12.99 | A |
| ATOM | 2756 | CG | LEU | A | 360 | 27.200 | 49.878 | 30.934 | 1.00 | 13.00 | A |
| ATOM | 2757 | CD1 | LEU | A | 360 | 27.222 | 50.806 | 29.731 | 1.00 | 14.39 | A |
| ATOM | 2758 | CD2 | LEU | A | 360 | 25.771 | 49.481 | 31.276 | 1.00 | 14.37 | A |
| ATOM | 2759 | C | LEU | A | 360 | 28.945 | 46.431 | 31.480 | 1.00 | 13.93 | A |
| ATOM | 2760 | O | LEU | A | 360 | 28.239 | 45.430 | 31.585 | 1.00 | 14.58 | A |
| ATOM | 2761 | N | PHE | A | 361 | 30.198 | 46.385 | 31.032 | 1.00 | 13.62 | A |
| ATOM | 2762 | CA | PHE | A | 361 | 30.825 | 45.130 | 30.618 | 1.00 | 13.96 | A |
| ATOM | 2763 | CB | PHE | A | 361 | 32.254 | 45.375 | 30.115 | 1.00 | 13.34 | A |
| ATOM | 2764 | CG | PHE | A | 361 | 32.338 | 46.176 | 28.843 | 1.00 | 12.91 | A |
| ATOM | 2765 | CD1 | PHE | A | 361 | 33.535 | 46.793 | 28.485 | 1.00 | 13.34 | A |
| ATOM | 2766 | CD2 | PHE | A | 361 | 31.238 | 46.310 | 28.002 | 1.00 | 12.77 | A |
| ATOM | 2767 | CE1 | PHE | A | 361 | 33.636 | 47.537 | 27.307 | 1.00 | 13.11 | A |
| ATOM | 2768 | CE2 | PHE | A | 361 | 31.329 | 47.052 | 26.819 | 1.00 | 12.47 | A |
| ATOM | 2769 | CZ | PHE | A | 361 | 32.531 | 47.665 | 26.474 | 1.00 | 12.92 | A |
| ATOM | 2770 | C | PHE | A | 361 | 30.889 | 44.134 | 31.774 | 1.00 | 14.49 | A |
| ATOM | 2771 | O | PHE | A | 361 | 30.559 | 42.959 | 31.614 | 1.00 | 14.15 | A |
| ATOM | 2772 | N | GLU | A | 362 | 31.329 | 44.601 | 32.939 | 1.00 | 15.28 | A |
| ATOM | 2773 | CA | GLU | A | 362 | 31.441 | 43.712 | 34.089 | 1.00 | 15.96 | A |
| ATOM | 2774 | CB | GLU | A | 362 | 31.969 | 44.459 | 35.316 | 1.00 | 18.25 | A |
| ATOM | 2775 | CG | GLU | A | 362 | 32.329 | 43.518 | 36.464 | 1.00 | 22.03 | A |
| ATOM | 2776 | CD | GLU | A | 362 | 32.708 | 44.244 | 37.740 | 1.00 | 24.32 | A |
| ATOM | 2777 | OE1 | GLU | A | 362 | 33.483 | 45.218 | 37.667 | 1.00 | 25.76 | A |
| ATOM | 2778 | OE2 | GLU | A | 362 | 32.238 | 43.829 | 38.821 | 1.00 | 26.40 | A |
| ATOM | 2779 | C | GLU | A | 362 | 30.095 | 43.084 | 34.427 | 1.00 | 15.98 | A |
| ATOM | 2780 | O | GLU | A | 362 | 30.010 | 41.886 | 34.691 | 1.00 | 16.22 | A |
| ATOM | 2781 | N | HIS | A | 363 | 29.044 | 43.894 | 34.417 | 1.00 | 15.25 | A |
| ATOM | 2782 | CA | HIS | A | 363 | 27.717 | 43.387 | 34.732 | 1.00 | 15.49 | A |
| ATOM | 2783 | CB | HIS | A | 363 | 26.727 | 44.537 | 34.914 | 1.00 | 16.71 | A |
| ATOM | 2784 | CG | HIS | A | 363 | 25.345 | 44.085 | 35.267 | 1.00 | 17.92 | A |
| ATOM | 2785 | CD2 | HIS | A | 363 | 24.204 | 44.054 | 34.539 | 1.00 | 18.79 | A |
| ATOM | 2786 | ND1 | HIS | A | 363 | 25.034 | 43.533 | 36.491 | 1.00 | 18.98 | A |
| ATOM | 2787 | CE1 | HIS | A | 363 | 23.761 | 43.180 | 36.502 | 1.00 | 18.19 | A |
| ATOM | 2788 | NE2 | HIS | A | 363 | 23.234 | 43.484 | 35.330 | 1.00 | 19.23 | A |
| ATOM | 2789 | C | HIS | A | 363 | 27.199 | 42.450 | 33.650 | 1.00 | 15.11 | A |
| ATOM | 2790 | O | HIS | A | 363 | 26.834 | 41.309 | 33.925 | 1.00 | 15.10 | A |
| ATOM | 2791 | N | ILE | A | 364 | 27.168 | 42.937 | 32.415 | 1.00 | 14.72 | A |
| ATOM | 2792 | CA | ILE | A | 364 | 26.679 | 42.146 | 31.297 | 1.00 | 14.52 | A |
| ATOM | 2793 | CB | ILE | A | 364 | 26.810 | 42.932 | 29.975 | 1.00 | 13.45 | A |
| ATOM | 2794 | CG2 | ILE | A | 364 | 26.416 | 42.043 | 28.795 | 1.00 | 13.94 | A |
| ATOM | 2795 | CG1 | ILE | A | 364 | 25.914 | 44.172 | 30.027 | 1.00 | 13.32 | A |
| ATOM | 2796 | CD1 | ILE | A | 364 | 26.170 | 45.165 | 28.912 | 1.00 | 13.44 | A |
| ATOM | 2797 | C | ILE | A | 364 | 27.388 | 40.807 | 31.141 | 1.00 | 14.67 | A |
| ATOM | 2798 | O | ILE | A | 364 | 26.741 | 39.769 | 30.996 | 1.00 | 15.31 | A |
| ATOM | 2799 | N | ASN | A | 365 | 28.715 | 40.822 | 31.175 | 1.00 | 15.38 | A |
| ATOM | 2800 | CA | ASN | A | 365 | 29.480 | 39.594 | 31.005 | 1.00 | 15.76 | A |
| ATOM | 2801 | CB | ASN | A | 365 | 30.957 | 39.917 | 30.771 | 1.00 | 15.37 | A |
| ATOM | 2802 | CG | ASN | A | 365 | 31.177 | 40.768 | 29.533 | 1.00 | 15.04 | A |
| ATOM | 2803 | OD1 | ASN | A | 365 | 30.276 | 40.935 | 28.710 | 1.00 | 14.73 | A |
| ATOM | 2804 | ND2 | ASN | A | 365 | 32.383 | 41.305 | 29.393 | 1.00 | 13.53 | A |
| ATOM | 2805 | C | ASN | A | 365 | 29.348 | 38.609 | 32.162 | 1.00 | 17.60 | A |
| ATOM | 2806 | O | ASN | A | 365 | 29.662 | 37.430 | 32.005 | 1.00 | 17.46 | A |
| ATOM | 2807 | N | SER | A | 366 | 28.879 | 39.087 | 33.313 | 1.00 | 19.18 | A |
| ATOM | 2808 | CA | SER | A | 366 | 28.717 | 38.226 | 34.484 | 1.00 | 21.16 | A |
| ATOM | 2809 | CB | SER | A | 366 | 29.114 | 38.976 | 35.761 | 1.00 | 21.81 | A |
| ATOM | 2810 | OG | SER | A | 366 | 28.220 | 40.039 | 36.033 | 1.00 | 22.86 | A |
| ATOM | 2811 | C | SER | A | 366 | 27.287 | 37.714 | 34.622 | 1.00 | 22.10 | A |
| ATOM | 2812 | O | SER | A | 366 | 27.020 | 36.810 | 35.414 | 1.00 | 22.66 | A |
| ATOM | 2813 | N | GLN | A | 367 | 26.373 | 38.299 | 33.854 | 1.00 | 23.09 | A |
| ATOM | 2814 | CA | GLN | A | 367 | 24.968 | 37.903 | 33.877 | 1.00 | 24.10 | A |
| ATOM | 2815 | CB | GLN | A | 367 | 24.064 | 39.132 | 33.749 | 1.00 | 25.62 | A |
| ATOM | 2816 | CG | GLN | A | 367 | 24.086 | 40.044 | 34.963 | 1.00 | 28.14 | A |
| ATOM | 2817 | CD | GLN | A | 367 | 23.515 | 39.374 | 36.195 | 1.00 | 29.20 | A |
| ATOM | 2818 | OE1 | GLN | A | 367 | 22.337 | 39.015 | 36.229 | 1.00 | 30.22 | A |
| ATOM | 2819 | NE2 | GLN | A | 367 | 24.348 | 39.197 | 37.215 | 1.00 | 30.01 | A |
| ATOM | 2820 | C | GLN | A | 367 | 24.693 | 36.942 | 32.726 | 1.00 | 23.83 | A |
| ATOM | 2821 | O | GLN | A | 367 | 24.413 | 37.363 | 31.604 | 1.00 | 23.81 | A |
| ATOM | 2822 | N | ALA | A | 368 | 24.765 | 35.649 | 33.019 | 1.00 | 23.74 | A |
| ATOM | 2823 | CA | ALA | A | 368 | 24.549 | 34.608 | 32.020 | 1.00 | 23.29 | A |
| ATOM | 2824 | CB | ALA | A | 368 | 24.533 | 33.244 | 32.700 | 1.00 | 23.52 | A |
| ATOM | 2825 | C | ALA | A | 368 | 23.293 | 34.771 | 31.165 | 1.00 | 23.34 | A |
| ATOM | 2826 | O | ALA | A | 368 | 23.315 | 34.476 | 29.970 | 1.00 | 22.74 | A |
| ATOM | 2827 | N | HIS | A | 369 | 22.202 | 35.238 | 31.767 | 1.00 | 23.07 | A |
| ATOM | 2828 | CA | HIS | A | 369 | 20.948 | 35.400 | 31.036 | 1.00 | 22.88 | A |
| ATOM | 2829 | CB | HIS | A | 369 | 19.854 | 35.924 | 31.977 | 1.00 | 24.69 | A |
| ATOM | 2830 | CG | HIS | A | 369 | 20.074 | 37.327 | 32.448 | 1.00 | 25.89 | A |
| ATOM | 2831 | CD2 | HIS | A | 369 | 20.553 | 37.810 | 33.619 | 1.00 | 26.56 | A |
| ATOM | 2832 | ND1 | HIS | A | 369 | 19.795 | 38.427 | 31.666 | 1.00 | 26.84 | A |
| ATOM | 2833 | CE1 | HIS | A | 369 | 20.090 | 39.527 | 32.335 | 1.00 | 26.51 | A |
| ATOM | 2834 | NE2 | HIS | A | 369 | 20.553 | 39.180 | 33.523 | 1.00 | 27.03 | A |
| ATOM | 2835 | C | HIS | A | 369 | 21.064 | 36.299 | 29.803 | 1.00 | 21.99 | A |
| ATOM | 2836 | O | HIS | A | 369 | 20.220 | 36.245 | 28.908 | 1.00 | 22.01 | A |
| ATOM | 2837 | N | PHE | A | 370 | 22.110 | 37.119 | 29.755 | 1.00 | 21.21 | A |
| ATOM | 2838 | CA | PHE | A | 370 | 22.329 | 38.006 | 28.615 | 1.00 | 19.78 | A |
| ATOM | 2839 | CB | PHE | A | 370 | 23.266 | 39.156 | 28.998 | 1.00 | 21.09 | A |
| ATOM | 2840 | CG | PHE | A | 370 | 22.592 | 40.279 | 29.736 | 1.00 | 21.42 | A |
| ATOM | 2841 | CD1 | PHE | A | 370 | 23.168 | 40.813 | 30.883 | 1.00 | 22.09 | A |
| ATOM | 2842 | CD2 | PHE | A | 370 | 21.401 | 40.826 | 29.267 | 1.00 | 22.35 | A |
| ATOM | 2843 | CE1 | PHE | A | 370 | 22.571 | 41.879 | 31.553 | 1.00 | 22.54 | A |
| ATOM | 2844 | CE2 | PHE | A | 370 | 20.795 | 41.894 | 29.931 | 1.00 | 22.87 | A |
| ATOM | 2845 | CZ | PHE | A | 370 | 21.384 | 42.420 | 31.076 | 1.00 | 22.18 | A |
| ATOM | 2846 | C | PHE | A | 370 | 22.953 | 37.235 | 27.456 | 1.00 | 18.22 | A |
| ATOM | 2847 | O | PHE | A | 370 | 22.673 | 37.515 | 26.288 | 1.00 | 17.08 | A |
| ATOM | 2848 | N | ASN | A | 371 | 23.802 | 36.268 | 27.791 | 1.00 | 16.98 | A |
| ATOM | 2849 | CA | ASN | A | 371 | 24.498 | 35.463 | 26.794 | 1.00 | 15.64 | A |
| ATOM | 2850 | CB | ASN | A | 371 | 23.500 | 34.613 | 26.004 | 1.00 | 16.15 | A |
| ATOM | 2851 | CG | ASN | A | 371 | 22.826 | 33.565 | 26.870 | 1.00 | 16.71 | A |
| ATOM | 2852 | OD1 | ASN | A | 371 | 23.483 | 32.668 | 27.401 | 1.00 | 18.32 | A |
| ATOM | 2853 | ND2 | ASN | A | 371 | 21.513 | 33.679 | 27.024 | 1.00 | 17.75 | A |
| ATOM | 2854 | C | ASN | A | 371 | 25.281 | 36.383 | 25.865 | 1.00 | 15.24 | A |
| ATOM | 2855 | O | ASN | A | 371 | 25.259 | 36.232 | 24.640 | 1.00 | 15.41 | A |
| ATOM | 2856 | N | VAL | A | 372 | 25.973 | 37.340 | 26.478 | 1.00 | 14.72 | A |
| ATOM | 2857 | CA | VAL | A | 372 | 26.784 | 38.318 | 25.763 | 1.00 | 14.11 | A |
| ATOM | 2858 | CB | VAL | A | 372 | 26.121 | 39.723 | 25.792 | 1.00 | 14.33 | A |
| ATOM | 2859 | CG1 | VAL | A | 372 | 27.095 | 40.780 | 25.264 | 1.00 | 13.94 | A |
| ATOM | 2860 | CG2 | VAL | A | 372 | 24.847 | 39.720 | 24.964 | 1.00 | 14.60 | A |
| ATOM | 2861 | C | VAL | A | 372 | 28.161 | 38.447 | 26.407 | 1.00 | 14.50 | A |
| ATOM | 2862 | O | VAL | A | 372 | 28.302 | 38.339 | 27.624 | 1.00 | 14.25 | A |
| ATOM | 2863 | N | GLN | A | 373 | 29.172 | 38.661 | 25.573 | 1.00 | 14.04 | A |
| ATOM | 2864 | CA | GLN | A | 373 | 30.539 | 38.877 | 26.031 | 1.00 | 14.11 | A |
| ATOM | 2865 | CB | GLN | A | 373 | 31.452 | 37.713 | 25.631 | 1.00 | 14.92 | A |
| ATOM | 2866 | CG | GLN | A | 373 | 32.933 | 37.914 | 25.971 | 1.00 | 16.67 | A |
| ATOM | 2867 | CD | GLN | A | 373 | 33.172 | 38.273 | 27.432 | 1.00 | 17.93 | A |
| ATOM | 2868 | OE1 | GLN | A | 373 | 32.509 | 37.754 | 28.329 | 1.00 | 19.36 | A |
| ATOM | 2869 | NE2 | GLN | A | 373 | 34.136 | 39.154 | 27.675 | 1.00 | 17.77 | A |
| ATOM | 2870 | C | GLN | A | 373 | 30.946 | 40.155 | 25.307 | 1.00 | 13.61 | A |
| ATOM | 2871 | O | GLN | A | 373 | 31.222 | 40.141 | 24.106 | 1.00 | 13.94 | A |
| ATOM | 2872 | N | ALA | A | 374 | 30.950 | 41.263 | 26.040 | 1.00 | 12.65 | A |
| ATOM | 2873 | CA | ALA | A | 374 | 31.281 | 42.558 | 25.466 | 1.00 | 12.16 | A |
| ATOM | 2874 | CB | ALA | A | 374 | 30.183 | 43.553 | 25.800 | 1.00 | 11.40 | A |
| ATOM | 2875 | C | ALA | A | 374 | 32.623 | 43.085 | 25.949 | 1.00 | 12.23 | A |
| ATOM | 2876 | O | ALA | A | 374 | 33.012 | 42.869 | 27.094 | 1.00 | 11.95 | A |
| ATOM | 2877 | N | GLN | A | 375 | 33.323 | 43.796 | 25.074 | 1.00 | 11.84 | A |
| ATOM | 2878 | CA | GLN | A | 375 | 34.619 | 44.356 | 25.432 | 1.00 | 12.49 | A |
| ATOM | 2879 | CB | GLN | A | 375 | 35.685 | 43.258 | 25.461 | 1.00 | 14.15 | A |
| ATOM | 2880 | CG | GLN | A | 375 | 35.804 | 42.468 | 24.160 | 1.00 | 17.09 | A |
| ATOM | 2881 | CD | GLN | A | 375 | 34.957 | 41.211 | 24.165 | 1.00 | 19.74 | A |
| ATOM | 2882 | OE1 | GLN | A | 375 | 35.187 | 40.302 | 24.964 | 1.00 | 21.17 | A |
| ATOM | 2883 | NE2 | GLN | A | 375 | 33.971 | 41.151 | 23.274 | 1.00 | 20.69 | A |
| ATOM | 2884 | C | GLN | A | 375 | 35.059 | 45.424 | 24.448 | 1.00 | 11.84 | A |
| ATOM | 2885 | O | GLN | A | 375 | 34.518 | 45.526 | 23.348 | 1.00 | 11.73 | A |
| ATOM | 2886 | N | PHE | A | 376 | 36.032 | 46.232 | 24.859 | 1.00 | 11.18 | A |
| ATOM | 2887 | CA | PHE | A | 376 | 36.579 | 47.245 | 23.969 | 1.00 | 10.74 | A |
| ATOM | 2888 | CB | PHE | A | 376 | 37.515 | 48.197 | 24.720 | 1.00 | 10.14 | A |
| ATOM | 2889 | CG | PHE | A | 376 | 36.814 | 49.112 | 25.681 | 1.00 | 10.91 | A |
| ATOM | 2890 | CD1 | PHE | A | 376 | 37.163 | 49.120 | 27.028 | 1.00 | 11.12 | A |
| ATOM | 2891 | CD2 | PHE | A | 376 | 35.807 | 49.971 | 25.243 | 1.00 | 11.24 | A |
| ATOM | 2892 | CE1 | PHE | A | 376 | 36.518 | 49.970 | 27.927 | 1.00 | 11.39 | A |
| ATOM | 2893 | CE2 | PHE | A | 376 | 35.158 | 50.825 | 26.138 | 1.00 | 11.60 | A |
| ATOM | 2894 | CZ | PHE | A | 376 | 35.516 | 50.821 | 27.482 | 1.00 | 11.57 | A |
| ATOM | 2895 | C | PHE | A | 376 | 37.386 | 46.450 | 22.954 | 1.00 | 11.25 | A |
| ATOM | 2896 | O | PHE | A | 376 | 38.015 | 45.448 | 23.297 | 1.00 | 10.76 | A |
| ATOM | 2897 | N | GLY | A | 377 | 37.364 | 46.882 | 21.702 | 1.00 | 11.05 | A |
| ATOM | 2898 | CA | GLY | A | 377 | 38.121 | 46.171 | 20.693 | 1.00 | 10.92 | A |
| ATOM | 2899 | C | GLY | A | 377 | 38.524 | 47.092 | 19.565 | 1.00 | 10.93 | A |
| ATOM | 2900 | O | GLY | A | 377 | 38.196 | 48.277 | 19.577 | 1.00 | 10.75 | A |
| ATOM | 2901 | N | THR | A | 378 | 39.257 | 46.549 | 18.600 | 1.00 | 10.49 | A |
| ATOM | 2902 | CA | THR | A | 378 | 39.674 | 47.333 | 17.447 | 1.00 | 11.19 | A |
| ATOM | 2903 | CB | THR | A | 378 | 41.187 | 47.214 | 17.182 | 1.00 | 11.98 | A |
| ATOM | 2904 | OG1 | THR | A | 378 | 41.508 | 45.860 | 16.845 | 1.00 | 11.89 | A |
| ATOM | 2905 | CG2 | THR | A | 378 | 41.981 | 47.640 | 18.411 | 1.00 | 12.75 | A |
| ATOM | 2906 | C | THR | A | 378 | 38.924 | 46.809 | 16.228 | 1.00 | 10.71 | A |
| ATOM | 2907 | O | THR | A | 378 | 38.233 | 45.788 | 16.296 | 1.00 | 10.80 | A |
| ATOM | 2908 | N | LEU | A | 379 | 39.059 | 47.509 | 15.111 | 1.00 | 10.53 | A |
| ATOM | 2909 | CA | LEU | A | 379 | 38.386 | 47.101 | 13.888 | 1.00 | 9.83 | A |
| ATOM | 2910 | CB | LEU | A | 379 | 38.605 | 48.160 | 12.803 | 1.00 | 9.72 | A |
| ATOM | 2911 | CG | LEU | A | 379 | 37.862 | 47.943 | 11.483 | 1.00 | 9.77 | A |
| ATOM | 2912 | CD1 | LEU | A | 379 | 36.357 | 47.930 | 11.748 | 1.00 | 10.37 | A |
| ATOM | 2913 | CD2 | LEU | A | 379 | 38.227 | 49.050 | 10.493 | 1.00 | 10.95 | A |
| ATOM | 2914 | C | LEU | A | 379 | 38.879 | 45.738 | 13.399 | 1.00 | 10.38 | A |
| ATOM | 2915 | O | LEU | A | 379 | 38.083 | 44.878 | 13.017 | 1.00 | 10.10 | A |
| ATOM | 2916 | N | GLN | A | 380 | 40.193 | 45.538 | 13.415 | 1.00 | 10.27 | A |
| ATOM | 2917 | CA | GLN | A | 380 | 40.764 | 44.274 | 12.964 | 1.00 | 10.67 | A |
| ATOM | 2918 | CB | GLN | A | 380 | 42.292 | 44.339 | 13.003 | 1.00 | 11.35 | A |
| ATOM | 2919 | CG | GLN | A | 380 | 42.975 | 43.119 | 12.410 | 1.00 | 14.06 | A |
| ATOM | 2920 | CD | GLN | A | 380 | 42.611 | 42.913 | 10.957 | 1.00 | 15.62 | A |
| ATOM | 2921 | OE1 | GLN | A | 380 | 42.678 | 43.846 | 10.155 | 1.00 | 16.75 | A |
| ATOM | 2922 | NE2 | GLN | A | 380 | 42.228 | 41.687 | 10.604 | 1.00 | 17.08 | A |
| ATOM | 2923 | C | GLN | A | 380 | 40.262 | 43.114 | 13.825 | 1.00 | 10.55 | A |
| ATOM | 2924 | O | GLN | A | 380 | 40.008 | 42.021 | 13.316 | 1.00 | 11.04 | A |
| ATOM | 2925 | N | GLU | A | 381 | 40.117 | 43.347 | 15.127 | 1.00 | 10.91 | A |
| ATOM | 2926 | CA | GLU | A | 381 | 39.633 | 42.298 | 16.016 | 1.00 | 11.75 | A |
| ATOM | 2927 | CB | GLU | A | 381 | 39.657 | 42.768 | 17.473 | 1.00 | 13.13 | A |
| ATOM | 2928 | CG | GLU | A | 381 | 41.063 | 43.061 | 17.981 | 1.00 | 16.73 | A |
| ATOM | 2929 | CD | GLU | A | 381 | 41.108 | 43.354 | 19.465 | 1.00 | 19.29 | A |
| ATOM | 2930 | OE1 | GLU | A | 381 | 40.266 | 44.141 | 19.941 | 1.00 | 18.94 | A |
| ATOM | 2931 | OE2 | GLU | A | 381 | 41.997 | 42.805 | 20.154 | 1.00 | 22.67 | A |
| ATOM | 2932 | C | GLU | A | 381 | 38.219 | 41.884 | 15.621 | 1.00 | 10.96 | A |
| ATOM | 2933 | O | GLU | A | 381 | 37.880 | 40.706 | 15.661 | 1.00 | 12.22 | A |
| ATOM | 2934 | N | TYR | A | 382 | 37.397 | 42.857 | 15.244 | 1.00 | 10.23 | A |
| ATOM | 2935 | CA | TYR | A | 382 | 36.037 | 42.562 | 14.821 | 1.00 | 9.86 | A |
| ATOM | 2936 | CB | TYR | A | 382 | 35.279 | 43.847 | 14.488 | 1.00 | 10.34 | A |
| ATOM | 2937 | CG | TYR | A | 382 | 33.967 | 43.581 | 13.784 | 1.00 | 9.83 | A |
| ATOM | 2938 | CD1 | TYR | A | 382 | 32.891 | 43.011 | 14.467 | 1.00 | 9.54 | A |
| ATOM | 2939 | CE1 | TYR | A | 382 | 31.702 | 42.700 | 13.809 | 1.00 | 9.35 | A |
| ATOM | 2940 | CD2 | TYR | A | 382 | 33.820 | 43.839 | 12.418 | 1.00 | 10.35 | A |
| ATOM | 2941 | CE2 | TYR | A | 382 | 32.634 | 43.528 | 11.750 | 1.00 | 10.43 | A |
| ATOM | 2942 | CZ | TYR | A | 382 | 31.581 | 42.957 | 12.453 | 1.00 | 10.11 | A |
| ATOM | 2943 | OH | TYR | A | 382 | 30.410 | 42.625 | 11.805 | 1.00 | 10.25 | A |
| ATOM | 2944 | C | TYR | A | 382 | 36.061 | 41.676 | 13.581 | 1.00 | 10.29 | A |
| ATOM | 2945 | O | TYR | A | 382 | 35.438 | 40.617 | 13.546 | 1.00 | 10.12 | A |
| ATOM | 2946 | N | PHE | A | 383 | 36.775 | 42.120 | 12.553 | 1.00 | 9.08 | A |
| ATOM | 2947 | CA | PHE | A | 383 | 36.852 | 41.353 | 11.320 | 1.00 | 9.53 | A |
| ATOM | 2948 | CB | PHE | A | 383 | 37.657 | 42.121 | 10.268 | 1.00 | 9.49 | A |
| ATOM | 2949 | CG | PHE | A | 383 | 36.898 | 43.262 | 9.638 | 1.00 | 9.73 | A |
| ATOM | 2950 | CD1 | PHE | A | 383 | 37.406 | 44.555 | 9.676 | 1.00 | 9.70 | A |
| ATOM | 2951 | CD2 | PHE | A | 383 | 35.678 | 43.038 | 9.003 | 1.00 | 10.22 | A |
| ATOM | 2952 | CE1 | PHE | A | 383 | 36.709 | 45.613 | 9.091 | 1.00 | 10.19 | A |
| ATOM | 2953 | CE2 | PHE | A | 383 | 34.971 | 44.088 | 8.415 | 1.00 | 11.04 | A |
| ATOM | 2954 | CZ | PHE | A | 383 | 35.490 | 45.378 | 8.459 | 1.00 | 11.28 | A |
| ATOM | 2955 | C | PHE | A | 383 | 37.441 | 39.961 | 11.528 | 1.00 | 9.84 | A |
| ATOM | 2956 | O | PHE | A | 383 | 36.971 | 38.993 | 10.926 | 1.00 | 10.29 | A |
| ATOM | 2957 | N | ASP | A | 384 | 38.466 | 39.851 | 12.370 | 1.00 | 10.93 | A |
| ATOM | 2958 | CA | ASP | A | 384 | 39.067 | 38.543 | 12.625 | 1.00 | 12.16 | A |
| ATOM | 2959 | CB | ASP | A | 384 | 40.239 | 38.655 | 13.608 | 1.00 | 13.63 | A |
| ATOM | 2960 | CG | ASP | A | 384 | 41.482 | 39.262 | 12.980 | 1.00 | 14.89 | A |
| ATOM | 2961 | OD1 | ASP | A | 384 | 41.572 | 39.309 | 11.735 | 1.00 | 16.13 | A |
| ATOM | 2962 | OD2 | ASP | A | 384 | 42.382 | 39.677 | 13.739 | 1.00 | 17.90 | A |
| ATOM | 2963 | C | ASP | A | 384 | 38.015 | 37.592 | 13.196 | 1.00 | 11.66 | A |
| ATOM | 2964 | O | ASP | A | 384 | 37.913 | 36.438 | 12.777 | 1.00 | 11.92 | A |
| ATOM | 2965 | N | ALA | A | 385 | 37.224 | 38.086 | 14.147 | 1.00 | 11.48 | A |
| ATOM | 2966 | CA | ALA | A | 385 | 36.181 | 37.280 | 14.771 | 1.00 | 11.44 | A |
| ATOM | 2967 | CB | ALA | A | 385 | 35.576 | 38.037 | 15.957 | 1.00 | 12.50 | A |
| ATOM | 2968 | C | ALA | A | 385 | 35.094 | 36.905 | 13.765 | 1.00 | 11.73 | A |
| ATOM | 2969 | O | ALA | A | 385 | 34.608 | 35.777 | 13.758 | 1.00 | 11.52 | A |
| ATOM | 2970 | N | VAL | A | 386 | 34.714 | 37.851 | 12.912 | 1.00 | 11.15 | A |
| ATOM | 2971 | CA | VAL | A | 386 | 33.694 | 37.585 | 11.904 | 1.00 | 11.93 | A |
| ATOM | 2972 | CB | VAL | A | 386 | 33.425 | 38.838 | 11.042 | 1.00 | 11.54 | A |
| ATOM | 2973 | CG1 | VAL | A | 386 | 32.580 | 38.471 | 9.826 | 1.00 | 12.39 | A |
| ATOM | 2974 | CG2 | VAL | A | 386 | 32.719 | 39.887 | 11.874 | 1.00 | 12.20 | A |
| ATOM | 2975 | C | VAL | A | 386 | 34.114 | 36.440 | 10.986 | 1.00 | 12.29 | A |
| ATOM | 2976 | O | VAL | A | 386 | 33.330 | 35.530 | 10.706 | 1.00 | 12.49 | A |
| ATOM | 2977 | N | HIS | A | 387 | 35.353 | 36.483 | 10.513 | 1.00 | 12.25 | A |
| ATOM | 2978 | CA | HIS | A | 387 | 35.832 | 35.440 | 9.619 | 1.00 | 12.83 | A |
| ATOM | 2979 | CB | HIS | A | 387 | 37.092 | 35.918 | 8.899 | 1.00 | 12.37 | A |
| ATOM | 2980 | CG | HIS | A | 387 | 36.827 | 37.039 | 7.942 | 1.00 | 13.26 | A |
| ATOM | 2981 | CD2 | HIS | A | 387 | 37.280 | 38.314 | 7.909 | 1.00 | 13.42 | A |
| ATOM | 2982 | ND1 | HIS | A | 387 | 35.950 | 36.916 | 6.886 | 1.00 | 14.23 | A |
| ATOM | 2983 | CE1 | HIS | A | 387 | 35.871 | 38.069 | 6.245 | 1.00 | 14.01 | A |
| ATOM | 2984 | NE2 | HIS | A | 387 | 36.668 | 38.934 | 6.846 | 1.00 | 13.21 | A |
| ATOM | 2985 | C | HIS | A | 387 | 36.049 | 34.111 | 10.331 | 1.00 | 13.93 | A |
| ATOM | 2986 | O | HIS | A | 387 | 35.995 | 33.050 | 9.708 | 1.00 | 13.04 | A |
| ATOM | 2987 | N | GLN | A | 388 | 36.281 | 34.166 | 11.639 | 1.00 | 14.93 | A |
| ATOM | 2988 | CA | GLN | A | 388 | 36.446 | 32.944 | 12.413 | 1.00 | 17.07 | A |
| ATOM | 2989 | CB | GLN | A | 388 | 36.914 | 33.272 | 13.833 | 1.00 | 18.68 | A |
| ATOM | 2990 | CG | GLN | A | 388 | 38.403 | 33.574 | 13.937 | 1.00 | 22.09 | A |
| ATOM | 2991 | CD | GLN | A | 388 | 38.764 | 34.330 | 15.206 | 1.00 | 24.01 | A |
| ATOM | 2992 | OE1 | GLN | A | 388 | 38.272 | 34.020 | 16.291 | 1.00 | 26.13 | A |
| ATOM | 2993 | NE2 | GLN | A | 388 | 39.637 | 35.324 | 15.075 | 1.00 | 25.42 | A |
| ATOM | 2994 | C | GLN | A | 388 | 35.081 | 32.260 | 12.443 | 1.00 | 17.56 | A |
| ATOM | 2995 | O | GLN | A | 388 | 34.983 | 31.039 | 12.342 | 1.00 | 17.57 | A |
| ATOM | 2996 | N | ALA | A | 389 | 34.027 | 33.063 | 12.566 | 1.00 | 18.15 | A |
| ATOM | 2997 | CA | ALA | A | 389 | 32.663 | 32.543 | 12.594 | 1.00 | 19.02 | A |
| ATOM | 2998 | CB | ALA | A | 389 | 31.688 | 33.647 | 12.990 | 1.00 | 18.44 | A |
| ATOM | 2999 | C | ALA | A | 389 | 32.310 | 31.996 | 11.215 | 1.00 | 20.03 | A |
| ATOM | 3000 | O | ALA | A | 389 | 31.630 | 30.976 | 11.093 | 1.00 | 20.84 | A |
| ATOM | 3001 | N | GLU | A | 390 | 32.776 | 32.688 | 10.178 | 1.00 | 20.86 | A |
| ATOM | 3002 | CA | GLU | A | 390 | 32.535 | 32.278 | 8.800 | 1.00 | 22.27 | A |
| ATOM | 3003 | CB | GLU | A | 390 | 33.109 | 33.326 | 7.839 | 1.00 | 21.67 | A |
| ATOM | 3004 | CG | GLU | A | 390 | 33.223 | 32.874 | 6.390 | 1.00 | 22.55 | A |
| ATOM | 3005 | CD | GLU | A | 390 | 33.774 | 33.964 | 5.491 | 1.00 | 22.57 | A |
| ATOM | 3006 | OE1 | GLU | A | 390 | 34.653 | 34.722 | 5.953 | 1.00 | 22.88 | A |
| ATOM | 3007 | OE2 | GLU | A | 390 | 33.341 | 34.057 | 4.323 | 1.00 | 23.67 | A |
| ATOM | 3008 | C | GLU | A | 390 | 33.181 | 30.920 | 8.544 | 1.00 | 23.75 | A |
| ATOM | 3009 | O | GLU | A | 390 | 32.563 | 30.028 | 7.960 | 1.00 | 23.58 | A |
| ATOM | 3010 | N | ARG | A | 391 | 34.426 | 30.769 | 8.984 | 1.00 | 25.19 | A |
| ATOM | 3011 | CA | ARG | A | 391 | 35.147 | 29.514 | 8.810 | 1.00 | 27.32 | A |
| ATOM | 3012 | CB | ARG | A | 391 | 36.609 | 29.675 | 9.233 | 1.00 | 28.48 | A |
| ATOM | 3013 | CG | ARG | A | 391 | 37.457 | 30.466 | 8.248 | 1.00 | 30.71 | A |
| ATOM | 3014 | CD | ARG | A | 391 | 38.898 | 30.573 | 8.723 | 1.00 | 32.35 | A |
| ATOM | 3015 | NE | ARG | A | 391 | 39.021 | 31.418 | 9.907 | 1.00 | 34.70 | A |
| ATOM | 3016 | CZ | ARG | A | 391 | 40.147 | 31.591 | 10.591 | 1.00 | 35.47 | A |
| ATOM | 3017 | NH1 | ARG | A | 391 | 41.259 | 30.973 | 10.212 | 1.00 | 36.35 | A |
| ATOM | 3018 | NH2 | ARG | A | 391 | 40.165 | 32.386 | 11.652 | 1.00 | 36.12 | A |
| ATOM | 3019 | C | ARG | A | 391 | 34.488 | 28.403 | 9.621 | 1.00 | 27.87 | A |
| ATOM | 3020 | O | ARG | A | 391 | 34.553 | 27.230 | 9.250 | 1.00 | 28.52 | A |
| ATOM | 3021 | N | ALA | A | 392 | 33.855 | 28.774 | 10.729 | 1.00 | 28.42 | A |
| ATOM | 3022 | CA | ALA | A | 392 | 33.174 | 27.798 | 11.572 | 1.00 | 29.34 | A |
| ATOM | 3023 | CB | ALA | A | 392 | 32.808 | 28.423 | 12.913 | 1.00 | 29.39 | A |
| ATOM | 3024 | C | ALA | A | 392 | 31.918 | 27.334 | 10.843 | 1.00 | 29.94 | A |
| ATOM | 3025 | O | ALA | A | 392 | 31.238 | 26.404 | 11.278 | 1.00 | 30.20 | A |
| ATOM | 3026 | N | GLY | A | 393 | 31.621 | 27.997 | 9.729 | 1.00 | 30.21 | A |
| ATOM | 3027 | CA | GLY | A | 393 | 30.459 | 27.647 | 8.936 | 1.00 | 30.73 | A |
| ATOM | 3028 | C | GLY | A | 393 | 29.137 | 28.156 | 9.476 | 1.00 | 30.90 | A |
| ATOM | 3029 | O | GLY | A | 393 | 28.089 | 27.583 | 9.186 | 1.00 | 30.72 | A |
| ATOM | 3030 | N | GLN | A | 394 | 29.167 | 29.230 | 10.258 | 1.00 | 31.23 | A |
| ATOM | 3031 | CA | GLN | A | 394 | 27.926 | 29.762 | 10.803 | 1.00 | 31.55 | A |
| ATOM | 3032 | CB | GLN | A | 394 | 28.125 | 30.254 | 12.243 | 1.00 | 32.81 | A |
| ATOM | 3033 | CG | GLN | A | 394 | 28.869 | 31.566 | 12.379 | 1.00 | 34.09 | A |
| ATOM | 3034 | CD | GLN | A | 394 | 28.756 | 32.150 | 13.777 | 1.00 | 34.34 | A |
| ATOM | 3035 | OE1 | GLN | A | 394 | 29.246 | 31.573 | 14.749 | 1.00 | 34.56 | A |
| ATOM | 3036 | NE2 | GLN | A | 394 | 28.099 | 33.299 | 13.883 | 1.00 | 33.78 | A |
| ATOM | 3037 | C | GLN | A | 394 | 27.373 | 30.892 | 9.944 | 1.00 | 30.68 | A |
| ATOM | 3038 | O | GLN | A | 394 | 26.308 | 31.434 | 10.233 | 1.00 | 31.18 | A |
| ATOM | 3039 | N | ALA | A | 395 | 28.091 | 31.242 | 8.880 | 1.00 | 29.36 | A |
| ATOM | 3040 | CA | ALA | A | 395 | 27.644 | 32.311 | 7.998 | 1.00 | 28.11 | A |
| ATOM | 3041 | CB | ALA | A | 395 | 27.939 | 33.667 | 8.639 | 1.00 | 28.51 | A |
| ATOM | 3042 | C | ALA | A | 395 | 28.263 | 32.253 | 6.607 | 1.00 | 27.07 | A |
| ATOM | 3043 | O | ALA | A | 395 | 29.413 | 31.849 | 6.434 | 1.00 | 26.55 | A |
| ATOM | 3044 | N | GLU | A | 396 | 27.472 | 32.656 | 5.618 | 1.00 | 26.11 | A |
| ATOM | 3045 | CA | GLU | A | 396 | 27.902 | 32.701 | 4.227 | 1.00 | 25.01 | A |
| ATOM | 3046 | CB | GLU | A | 396 | 27.081 | 31.731 | 3.372 | 1.00 | 28.19 | A |
| ATOM | 3047 | CG | GLU | A | 396 | 26.403 | 30.616 | 4.156 | 1.00 | 32.27 | A |
| ATOM | 3048 | CD | GLU | A | 396 | 25.087 | 31.055 | 4.780 | 1.00 | 34.46 | A |
| ATOM | 3049 | OE1 | GLU | A | 396 | 25.079 | 32.035 | 5.558 | 1.00 | 35.47 | A |
| ATOM | 3050 | OE2 | GLU | A | 396 | 24.054 | 30.414 | 4.491 | 1.00 | 35.90 | A |
| ATOM | 3051 | C | GLU | A | 396 | 27.610 | 34.134 | 3.804 | 1.00 | 22.25 | A |
| ATOM | 3052 | O | GLU | A | 396 | 26.559 | 34.675 | 4.147 | 1.00 | 22.54 | A |
| ATOM | 3053 | N | PHE | A | 397 | 28.528 | 34.756 | 3.075 | 1.00 | 18.45 | A |
| ATOM | 3054 | CA | PHE | A | 397 | 28.313 | 36.134 | 2.661 | 1.00 | 15.02 | A |
| ATOM | 3055 | CB | PHE | A | 397 | 29.530 | 36.988 | 3.024 | 1.00 | 14.03 | A |
| ATOM | 3056 | CG | PHE | A | 397 | 29.830 | 37.014 | 4.493 | 1.00 | 11.87 | A |
| ATOM | 3057 | CD1 | PHE | A | 397 | 30.916 | 36.315 | 5.008 | 1.00 | 12.64 | A |
| ATOM | 3058 | CD2 | PHE | A | 397 | 29.010 | 37.718 | 5.367 | 1.00 | 11.17 | A |
| ATOM | 3059 | CE1 | PHE | A | 397 | 31.181 | 36.318 | 6.379 | 1.00 | 12.60 | A |
| ATOM | 3060 | CE2 | PHE | A | 397 | 29.267 | 37.725 | 6.737 | 1.00 | 11.98 | A |
| ATOM | 3061 | CZ | PHE | A | 397 | 30.354 | 37.024 | 7.240 | 1.00 | 12.05 | A |
| ATOM | 3062 | C | PHE | A | 397 | 27.998 | 36.295 | 1.181 | 1.00 | 13.50 | A |
| ATOM | 3063 | O | PHE | A | 397 | 28.549 | 35.592 | 0.334 | 1.00 | 13.62 | A |
| ATOM | 3064 | N | PRO | A | 398 | 27.090 | 37.227 | 0.857 | 1.00 | 11.54 | A |
| ATOM | 3065 | CD | PRO | A | 398 | 26.315 | 38.064 | 1.791 | 1.00 | 11.74 | A |
| ATOM | 3066 | CA | PRO | A | 398 | 26.694 | 37.491 | −0.526 | 1.00 | 11.19 | A |
| ATOM | 3067 | CB | PRO | A | 398 | 25.417 | 38.302 | −0.361 | 1.00 | 11.18 | A |
| ATOM | 3068 | CG | PRO | A | 398 | 25.711 | 39.107 | 0.873 | 1.00 | 10.73 | A |
| ATOM | 3069 | C | PRO | A | 398 | 27.765 | 38.267 | −1.284 | 1.00 | 10.60 | A |
| ATOM | 3070 | O | PRO | A | 398 | 28.606 | 38.941 | −0.684 | 1.00 | 10.78 | A |
| ATOM | 3071 | N | THR | A | 399 | 27.729 | 38.150 | −2.604 | 1.00 | 10.12 | A |
| ATOM | 3072 | CA | THR | A | 399 | 28.663 | 38.849 | −3.473 | 1.00 | 9.51 | A |
| ATOM | 3073 | CB | THR | A | 399 | 29.072 | 37.968 | −4.661 | 1.00 | 9.66 | A |
| ATOM | 3074 | OG1 | THR | A | 399 | 27.897 | 37.527 | −5.358 | 1.00 | 11.62 | A |
| ATOM | 3075 | CG2 | THR | A | 399 | 29.846 | 36.754 | −4.165 | 1.00 | 10.14 | A |
| ATOM | 3076 | C | THR | A | 399 | 27.932 | 40.085 | −3.980 | 1.00 | 9.58 | A |
| ATOM | 3077 | O | THR | A | 399 | 26.713 | 40.071 | −4.128 | 1.00 | 9.11 | A |
| ATOM | 3078 | N | LEU | A | 400 | 28.671 | 41.155 | −4.245 | 1.00 | 8.68 | A |
| ATOM | 3079 | CA | LEU | A | 400 | 28.042 | 42.383 | −4.705 | 1.00 | 8.59 | A |
| ATOM | 3080 | CB | LEU | A | 400 | 27.656 | 43.250 | −3.492 | 1.00 | 9.30 | A |
| ATOM | 3081 | CG | LEU | A | 400 | 27.037 | 44.646 | −3.685 | 1.00 | 8.82 | A |
| ATOM | 3082 | CD1 | LEU | A | 400 | 26.267 | 45.023 | −2.424 | 1.00 | 10.19 | A |
| ATOM | 3083 | CD2 | LEU | A | 400 | 28.113 | 45.690 | −3.995 | 1.00 | 9.06 | A |
| ATOM | 3084 | C | LEU | A | 400 | 28.958 | 43.169 | −5.618 | 1.00 | 8.59 | A |
| ATOM | 3085 | O | LEU | A | 400 | 30.178 | 43.136 | −5.467 | 1.00 | 8.00 | A |
| ATOM | 3086 | N | SER | A | 401 | 28.359 | 43.857 | −6.584 | 1.00 | 8.67 | A |
| ATOM | 3087 | CA | SER | A | 401 | 29.117 | 44.717 | −7.481 | 1.00 | 8.41 | A |
| ATOM | 3088 | CB | SER | A | 401 | 29.371 | 44.052 | −8.838 | 1.00 | 9.45 | A |
| ATOM | 3089 | OG | SER | A | 401 | 28.244 | 44.125 | −9.694 | 1.00 | 9.90 | A |
| ATOM | 3090 | C | SER | A | 401 | 28.281 | 45.975 | −7.661 | 1.00 | 8.49 | A |
| ATOM | 3091 | O | SER | A | 401 | 27.063 | 45.955 | −7.462 | 1.00 | 7.41 | A |
| ATOM | 3092 | N | GLY | A | 402 | 28.943 | 47.069 | −8.021 | 1.00 | 7.28 | A |
| ATOM | 3093 | CA | GLY | A | 402 | 28.252 | 48.332 | −8.222 | 1.00 | 7.95 | A |
| ATOM | 3094 | C | GLY | A | 402 | 28.860 | 49.410 | −7.346 | 1.00 | 8.13 | A |
| ATOM | 3095 | O | GLY | A | 402 | 29.905 | 49.190 | −6.724 | 1.00 | 8.75 | A |
| ATOM | 3096 | N | ASP | A | 403 | 28.220 | 50.575 | −7.297 | 1.00 | 7.93 | A |
| ATOM | 3097 | CA | ASP | A | 403 | 28.720 | 51.670 | −6.473 | 1.00 | 8.03 | A |
| ATOM | 3098 | CB | ASP | A | 403 | 29.229 | 52.815 | −7.360 | 1.00 | 8.33 | A |
| ATOM | 3099 | CG | ASP | A | 403 | 28.112 | 53.578 | −8.037 | 1.00 | 10.13 | A |
| ATOM | 3100 | OD1 | ASP | A | 403 | 26.994 | 53.039 | −8.160 | 1.00 | 12.44 | A |
| ATOM | 3101 | OD2 | ASP | A | 403 | 28.363 | 54.724 | −8.460 | 1.00 | 12.35 | A |
| ATOM | 3102 | C | ASP | A | 403 | 27.623 | 52.162 | −5.541 | 1.00 | 8.91 | A |
| ATOM | 3103 | O | ASP | A | 403 | 26.516 | 51.609 | −5.521 | 1.00 | 8.76 | A |
| ATOM | 3104 | N | PHE | A | 404 | 27.933 | 53.192 | −4.761 | 1.00 | 7.11 | A |
| ATOM | 3105 | CA | PHE | A | 404 | 26.967 | 53.740 | −3.827 | 1.00 | 7.03 | A |
| ATOM | 3106 | CB | PHE | A | 404 | 27.338 | 53.341 | −2.391 | 1.00 | 7.25 | A |
| ATOM | 3107 | CG | PHE | A | 404 | 27.292 | 51.852 | −2.163 | 1.00 | 7.96 | A |
| ATOM | 3108 | CD1 | PHE | A | 404 | 28.457 | 51.092 | −2.183 | 1.00 | 8.45 | A |
| ATOM | 3109 | CD2 | PHE | A | 404 | 26.070 | 51.203 | −1.994 | 1.00 | 8.79 | A |
| ATOM | 3110 | CE1 | PHE | A | 404 | 28.407 | 49.702 | −2.042 | 1.00 | 8.99 | A |
| ATOM | 3111 | CE2 | PHE | A | 404 | 26.010 | 49.813 | −1.853 | 1.00 | 8.46 | A |
| ATOM | 3112 | CZ | PHE | A | 404 | 27.180 | 49.064 | −1.877 | 1.00 | 9.11 | A |
| ATOM | 3113 | C | PHE | A | 404 | 26.785 | 55.247 | −3.957 | 1.00 | 5.97 | A |
| ATOM | 3114 | O | PHE | A | 404 | 26.922 | 55.998 | −2.994 | 1.00 | 6.84 | A |
| ATOM | 3115 | N | PHE | A | 405 | 26.470 | 55.664 | −5.180 | 1.00 | 7.41 | A |
| ATOM | 3116 | CA | PHE | A | 405 | 26.190 | 57.059 | −5.512 | 1.00 | 8.36 | A |
| ATOM | 3117 | CB | PHE | A | 405 | 27.228 | 57.624 | −6.494 | 1.00 | 7.18 | A |
| ATOM | 3118 | CG | PHE | A | 405 | 28.618 | 57.725 | −5.930 | 1.00 | 7.06 | A |
| ATOM | 3119 | CD1 | PHE | A | 405 | 29.676 | 57.062 | −6.542 | 1.00 | 8.48 | A |
| ATOM | 3120 | CD2 | PHE | A | 405 | 28.875 | 58.492 | −4.797 | 1.00 | 7.69 | A |
| ATOM | 3121 | CE1 | PHE | A | 405 | 30.975 | 57.159 | −6.033 | 1.00 | 7.72 | A |
| ATOM | 3122 | CE2 | PHE | A | 405 | 30.171 | 58.595 | −4.280 | 1.00 | 7.86 | A |
| ATOM | 3123 | CZ | PHE | A | 405 | 31.221 | 57.926 | −4.901 | 1.00 | 7.74 | A |
| ATOM | 3124 | C | PHE | A | 405 | 24.832 | 56.989 | −6.216 | 1.00 | 9.74 | A |
| ATOM | 3125 | O | PHE | A | 405 | 24.548 | 56.000 | −6.894 | 1.00 | 11.55 | A |
| ATOM | 3126 | N | THR | A | 406 | 23.989 | 58.009 | −6.080 | 1.00 | 10.20 | A |
| ATOM | 3127 | CA | THR | A | 406 | 24.266 | 59.205 | −5.302 | 1.00 | 9.22 | A |
| ATOM | 3128 | CB | THR | A | 406 | 23.738 | 60.449 | −6.047 | 1.00 | 8.18 | A |
| ATOM | 3129 | OG1 | THR | A | 406 | 24.579 | 60.693 | −7.179 | 1.00 | 10.10 | A |
| ATOM | 3130 | CG2 | THR | A | 406 | 23.724 | 61.677 | −5.149 | 1.00 | 8.41 | A |
| ATOM | 3131 | C | THR | A | 406 | 23.638 | 59.107 | −3.918 | 1.00 | 9.23 | A |
| ATOM | 3132 | O | THR | A | 406 | 22.478 | 58.721 | −3.761 | 1.00 | 10.33 | A |
| ATOM | 3133 | N | TYR | A | 407 | 24.433 | 59.462 | −2.918 | 1.00 | 9.08 | A |
| ATOM | 3134 | CA | TYR | A | 407 | 24.030 | 59.418 | −1.520 | 1.00 | 9.23 | A |
| ATOM | 3135 | CB | TYR | A | 407 | 25.276 | 59.650 | −0.660 | 1.00 | 9.83 | A |
| ATOM | 3136 | CG | TYR | A | 407 | 25.047 | 59.828 | 0.824 | 1.00 | 10.11 | A |
| ATOM | 3137 | CD1 | TYR | A | 407 | 24.571 | 58.779 | 1.614 | 1.00 | 9.79 | A |
| ATOM | 3138 | CE1 | TYR | A | 407 | 24.448 | 58.918 | 2.997 | 1.00 | 10.83 | A |
| ATOM | 3139 | CD2 | TYR | A | 407 | 25.383 | 61.024 | 1.453 | 1.00 | 9.98 | A |
| ATOM | 3140 | CE2 | TYR | A | 407 | 25.265 | 61.175 | 2.833 | 1.00 | 9.66 | A |
| ATOM | 3141 | CZ | TYR | A | 407 | 24.802 | 60.119 | 3.600 | 1.00 | 9.91 | A |
| ATOM | 3142 | OH | TYR | A | 407 | 24.732 | 60.257 | 4.967 | 1.00 | 10.51 | A |
| ATOM | 3143 | C | TYR | A | 407 | 22.954 | 60.423 | −1.129 | 1.00 | 10.35 | A |
| ATOM | 3144 | O | TYR | A | 407 | 22.947 | 61.559 | −1.593 | 1.00 | 10.39 | A |
| ATOM | 3145 | N | ALA | A | 408 | 22.043 | 59.976 | −0.272 | 1.00 | 10.77 | A |
| ATOM | 3146 | CA | ALA | A | 408 | 20.980 | 60.809 | 0.279 | 1.00 | 10.56 | A |
| ATOM | 3147 | CB | ALA | A | 408 | 19.652 | 60.549 | −0.432 | 1.00 | 12.09 | A |
| ATOM | 3148 | C | ALA | A | 408 | 20.900 | 60.360 | 1.732 | 1.00 | 10.90 | A |
| ATOM | 3149 | O | ALA | A | 408 | 20.748 | 59.165 | 1.999 | 1.00 | 10.68 | A |
| ATOM | 3150 | N | ASP | A | 409 | 21.031 | 61.296 | 2.671 | 1.00 | 10.55 | A |
| ATOM | 3151 | CA | ASP | A | 409 | 20.971 | 60.933 | 4.084 | 1.00 | 11.69 | A |
| ATOM | 3152 | CB | ASP | A | 409 | 21.946 | 61.795 | 4.920 | 1.00 | 11.33 | A |
| ATOM | 3153 | CG | ASP | A | 409 | 21.643 | 63.292 | 4.869 | 1.00 | 10.60 | A |
| ATOM | 3154 | OD1 | ASP | A | 409 | 21.024 | 63.770 | 3.898 | 1.00 | 11.47 | A |
| ATOM | 3155 | OD2 | ASP | A | 409 | 22.065 | 64.004 | 5.811 | 1.00 | 12.60 | A |
| ATOM | 3156 | C | ASP | A | 409 | 19.549 | 60.987 | 4.641 | 1.00 | 12.81 | A |
| ATOM | 3157 | O | ASP | A | 409 | 19.258 | 60.388 | 5.680 | 1.00 | 14.02 | A |
| ATOM | 3158 | N | ARG | A | 410 | 18.662 | 61.683 | 3.935 | 1.00 | 13.88 | A |
| ATOM | 3159 | CA | ARG | A | 410 | 17.263 | 61.785 | 4.343 | 1.00 | 15.32 | A |
| ATOM | 3160 | CB | ARG | A | 410 | 17.128 | 62.574 | 5.652 | 1.00 | 17.28 | A |
| ATOM | 3161 | CG | ARG | A | 410 | 17.637 | 64.009 | 5.620 | 1.00 | 20.04 | A |
| ATOM | 3162 | CD | ARG | A | 410 | 17.627 | 64.579 | 7.035 | 1.00 | 23.14 | A |
| ATOM | 3163 | NE | ARG | A | 410 | 18.215 | 65.913 | 7.135 | 1.00 | 26.24 | A |
| ATOM | 3164 | CZ | ARG | A | 410 | 17.598 | 67.038 | 6.787 | 1.00 | 28.17 | A |
| ATOM | 3165 | NH1 | ARG | A | 410 | 16.359 | 67.004 | 6.310 | 1.00 | 28.33 | A |
| ATOM | 3166 | NH2 | ARG | A | 410 | 18.219 | 68.201 | 6.924 | 1.00 | 28.66 | A |
| ATOM | 3167 | C | ARG | A | 410 | 16.407 | 62.426 | 3.256 | 1.00 | 15.93 | A |
| ATOM | 3168 | O | ARG | A | 410 | 16.911 | 63.174 | 2.416 | 1.00 | 15.40 | A |
| ATOM | 3169 | N | SER | A | 411 | 15.115 | 62.110 | 3.282 | 1.00 | 15.97 | A |
| ATOM | 3170 | CA | SER | A | 411 | 14.141 | 62.624 | 2.320 | 1.00 | 16.02 | A |
| ATOM | 3171 | CB | SER | A | 411 | 13.602 | 63.984 | 2.787 | 1.00 | 17.79 | A |
| ATOM | 3172 | OG | SER | A | 411 | 14.647 | 64.906 | 3.039 | 1.00 | 20.12 | A |
| ATOM | 3173 | C | SER | A | 411 | 14.673 | 62.723 | 0.892 | 1.00 | 15.26 | A |
| ATOM | 3174 | O | SER | A | 411 | 15.124 | 61.725 | 0.322 | 1.00 | 15.04 | A |
| ATOM | 3175 | N | ASP | A | 412 | 14.615 | 63.919 | 0.313 | 1.00 | 13.50 | A |
| ATOM | 3176 | CA | ASP | A | 412 | 15.089 | 64.133 | −1.052 | 1.00 | 13.01 | A |
| ATOM | 3177 | CB | ASP | A | 412 | 14.052 | 64.937 | −1.848 | 1.00 | 13.83 | A |
| ATOM | 3178 | CG | ASP | A | 412 | 13.911 | 66.369 | −1.354 | 1.00 | 14.01 | A |
| ATOM | 3179 | OD1 | ASP | A | 412 | 14.388 | 66.672 | −0.241 | 1.00 | 13.33 | A |
| ATOM | 3180 | OD2 | ASP | A | 412 | 13.310 | 67.196 | −2.077 | 1.00 | 15.12 | A |
| ATOM | 3181 | C | ASP | A | 412 | 16.423 | 64.871 | −1.054 | 1.00 | 12.08 | A |
| ATOM | 3182 | O | ASP | A | 412 | 16.822 | 65.439 | −2.071 | 1.00 | 12.37 | A |
| ATOM | 3183 | N | ASN | A | 413 | 17.108 | 64.857 | 0.087 | 1.00 | 11.65 | A |
| ATOM | 3184 | CA | ASN | A | 413 | 18.393 | 65.538 | 0.215 | 1.00 | 11.09 | A |
| ATOM | 3185 | CB | ASN | A | 413 | 18.703 | 65.851 | 1.683 | 1.00 | 11.71 | A |
| ATOM | 3186 | CG | ASN | A | 413 | 17.804 | 66.933 | 2.271 | 1.00 | 11.32 | A |
| ATOM | 3187 | OD1 | ASN | A | 413 | 18.069 | 67.432 | 3.364 | 1.00 | 12.21 | A |
| ATOM | 3188 | ND2 | ASN | A | 413 | 16.742 | 67.293 | 1.559 | 1.00 | 11.39 | A |
| ATOM | 3189 | C | ASN | A | 413 | 19.536 | 64.709 | −0.365 | 1.00 | 11.02 | A |
| ATOM | 3190 | O | ASN | A | 413 | 20.261 | 64.039 | 0.373 | 1.00 | 11.48 | A |
| ATOM | 3191 | N | TYR | A | 414 | 19.688 | 64.758 | −1.687 | 1.00 | 10.66 | A |
| ATOM | 3192 | CA | TYR | A | 414 | 20.757 | 64.029 | −2.367 | 1.00 | 10.00 | A |
| ATOM | 3193 | CB | TYR | A | 414 | 20.335 | 63.638 | −3.787 | 1.00 | 10.30 | A |
| ATOM | 3194 | CG | TYR | A | 414 | 19.333 | 62.505 | −3.824 | 1.00 | 10.55 | A |
| ATOM | 3195 | CD1 | TYR | A | 414 | 17.974 | 62.741 | −3.616 | 1.00 | 10.75 | A |
| ATOM | 3196 | CE1 | TYR | A | 414 | 17.055 | 61.688 | −3.606 | 1.00 | 10.98 | A |
| ATOM | 3197 | CD2 | TYR | A | 414 | 19.752 | 61.191 | −4.025 | 1.00 | 10.20 | A |
| ATOM | 3198 | CE2 | TYR | A | 414 | 18.844 | 60.134 | −4.016 | 1.00 | 10.67 | A |
| ATOM | 3199 | CZ | TYR | A | 414 | 17.502 | 60.390 | −3.806 | 1.00 | 10.80 | A |
| ATOM | 3200 | OH | TYR | A | 414 | 16.609 | 59.340 | −3.796 | 1.00 | 10.77 | A |
| ATOM | 3201 | C | TYR | A | 414 | 22.008 | 64.899 | −2.419 | 1.00 | 10.13 | A |
| ATOM | 3202 | O | TYR | A | 414 | 21.942 | 66.075 | −2.778 | 1.00 | 10.53 | A |
| ATOM | 3203 | N | TRP | A | 415 | 23.142 | 64.302 | −2.072 | 1.00 | 9.52 | A |
| ATOM | 3204 | CA | TRP | A | 415 | 24.416 | 65.010 | −2.030 | 1.00 | 9.26 | A |
| ATOM | 3205 | CB | TRP | A | 415 | 25.320 | 64.366 | −0.971 | 1.00 | 9.67 | A |
| ATOM | 3206 | CG | TRP | A | 415 | 24.786 | 64.442 | 0.441 | 1.00 | 9.03 | A |
| ATOM | 3207 | CD2 | TRP | A | 415 | 25.545 | 64.689 | 1.632 | 1.00 | 9.61 | A |
| ATOM | 3208 | CE2 | TRP | A | 415 | 24.650 | 64.605 | 2.725 | 1.00 | 9.55 | A |
| ATOM | 3209 | CE3 | TRP | A | 415 | 26.896 | 64.972 | 1.882 | 1.00 | 9.25 | A |
| ATOM | 3210 | CD1 | TRP | A | 415 | 23.494 | 64.225 | 0.851 | 1.00 | 9.72 | A |
| ATOM | 3211 | NE1 | TRP | A | 415 | 23.408 | 64.321 | 2.220 | 1.00 | 9.57 | A |
| ATOM | 3212 | CZ2 | TRP | A | 415 | 25.064 | 64.794 | 4.051 | 1.00 | 9.83 | A |
| ATOM | 3213 | CZ3 | TRP | A | 415 | 27.309 | 65.158 | 3.201 | 1.00 | 9.05 | A |
| ATOM | 3214 | CH2 | TRP | A | 415 | 26.393 | 65.068 | 4.270 | 1.00 | 9.98 | A |
| ATOM | 3215 | C | TRP | A | 415 | 25.140 | 65.041 | −3.372 | 1.00 | 9.53 | A |
| ATOM | 3216 | O | TRP | A | 415 | 26.251 | 64.528 | −3.495 | 1.00 | 10.20 | A |
| ATOM | 3217 | N | SER | A | 416 | 24.514 | 65.639 | −4.379 | 1.00 | 9.04 | A |
| ATOM | 3218 | CA | SER | A | 416 | 25.143 | 65.724 | −5.690 | 1.00 | 8.92 | A |
| ATOM | 3219 | CB | SER | A | 416 | 24.144 | 65.357 | −6.798 | 1.00 | 8.51 | A |
| ATOM | 3220 | OG | SER | A | 416 | 22.870 | 65.939 | −6.574 | 1.00 | 8.91 | A |
| ATOM | 3221 | C | SER | A | 416 | 25.728 | 67.110 | −5.933 | 1.00 | 8.58 | A |
| ATOM | 3222 | O | SER | A | 416 | 26.388 | 67.346 | −6.939 | 1.00 | 8.72 | A |
| ATOM | 3223 | N | GLY | A | 417 | 25.504 | 68.023 | −4.995 | 1.00 | 8.72 | A |
| ATOM | 3224 | CA | GLY | A | 417 | 26.035 | 69.361 | −5.155 | 1.00 | 9.16 | A |
| ATOM | 3225 | C | GLY | A | 417 | 27.549 | 69.389 | −5.084 | 1.00 | 8.20 | A |
| ATOM | 3226 | O | GLY | A | 417 | 28.194 | 70.109 | −5.846 | 1.00 | 8.68 | A |
| ATOM | 3227 | N | TYR | A | 418 | 28.125 | 68.591 | −4.187 | 1.00 | 8.03 | A |
| ATOM | 3228 | CA | TYR | A | 418 | 29.575 | 68.583 | −4.018 | 1.00 | 7.87 | A |
| ATOM | 3229 | CB | TYR | A | 418 | 29.955 | 67.891 | −2.699 | 1.00 | 7.48 | A |
| ATOM | 3230 | CG | TYR | A | 418 | 30.086 | 66.390 | −2.762 | 1.00 | 7.93 | A |
| ATOM | 3231 | CD1 | TYR | A | 418 | 31.328 | 65.794 | −2.973 | 1.00 | 7.95 | A |
| ATOM | 3232 | CE1 | TYR | A | 418 | 31.467 | 64.414 | −2.997 | 1.00 | 7.84 | A |
| ATOM | 3233 | CD2 | TYR | A | 418 | 28.978 | 65.563 | −2.583 | 1.00 | 8.11 | A |
| ATOM | 3234 | CE2 | TYR | A | 418 | 29.104 | 64.177 | −2.608 | 1.00 | 8.06 | A |
| ATOM | 3235 | CZ | TYR | A | 418 | 30.354 | 63.611 | −2.813 | 1.00 | 7.96 | A |
| ATOM | 3236 | OH | TYR | A | 418 | 30.501 | 62.248 | −2.817 | 1.00 | 8.02 | A |
| ATOM | 3237 | C | TYR | A | 418 | 30.346 | 67.988 | −5.199 | 1.00 | 7.88 | A |
| ATOM | 3238 | O | TYR | A | 418 | 31.576 | 67.989 | −5.208 | 1.00 | 7.86 | A |
| ATOM | 3239 | N | TYR | A | 419 | 29.627 | 67.477 | −6.194 | 1.00 | 7.67 | A |
| ATOM | 3240 | CA | TYR | A | 419 | 30.282 | 66.952 | −7.388 | 1.00 | 7.72 | A |
| ATOM | 3241 | CB | TYR | A | 419 | 29.290 | 66.179 | −8.265 | 1.00 | 7.93 | A |
| ATOM | 3242 | CG | TYR | A | 419 | 28.676 | 64.947 | −7.644 | 1.00 | 7.53 | A |
| ATOM | 3243 | CD1 | TYR | A | 419 | 27.487 | 64.416 | −8.152 | 1.00 | 7.43 | A |
| ATOM | 3244 | CE1 | TYR | A | 419 | 26.926 | 63.261 | −7.619 | 1.00 | 8.40 | A |
| ATOM | 3245 | CD2 | TYR | A | 419 | 29.289 | 64.285 | −6.579 | 1.00 | 7.84 | A |
| ATOM | 3246 | CE2 | TYR | A | 419 | 28.735 | 63.123 | −6.041 | 1.00 | 8.37 | A |
| ATOM | 3247 | CZ | TYR | A | 419 | 27.553 | 62.619 | −6.567 | 1.00 | 8.35 | A |
| ATOM | 3248 | OH | TYR | A | 419 | 26.995 | 61.474 | −6.047 | 1.00 | 8.31 | A |
| ATOM | 3249 | C | TYR | A | 419 | 30.803 | 68.150 | −8.190 | 1.00 | 7.72 | A |
| ATOM | 3250 | O | TYR | A | 419 | 31.616 | 67.988 | −9.103 | 1.00 | 8.10 | A |
| ATOM | 3251 | N | THR | A | 420 | 30.338 | 69.348 | −7.832 | 1.00 | 7.79 | A |
| ATOM | 3252 | CA | THR | A | 420 | 30.718 | 70.574 | −8.531 | 1.00 | 7.63 | A |
| ATOM | 3253 | CB | THR | A | 420 | 29.503 | 71.111 | −9.333 | 1.00 | 7.52 | A |
| ATOM | 3254 | OG1 | THR | A | 420 | 29.016 | 70.077 | −10.199 | 1.00 | 8.69 | A |
| ATOM | 3255 | CG2 | THR | A | 420 | 29.889 | 72.330 | −10.171 | 1.00 | 9.65 | A |
| ATOM | 3256 | C | THR | A | 420 | 31.261 | 71.712 | −7.658 | 1.00 | 8.05 | A |
| ATOM | 3257 | O | THR | A | 420 | 32.050 | 72.527 | −8.131 | 1.00 | 8.13 | A |
| ATOM | 3258 | N | SER | A | 421 | 30.844 | 71.771 | −6.396 | 1.00 | 8.23 | A |
| ATOM | 3259 | CA | SER | A | 421 | 31.277 | 72.840 | −5.491 | 1.00 | 8.39 | A |
| ATOM | 3260 | CB | SER | A | 421 | 30.894 | 72.488 | −4.054 | 1.00 | 8.23 | A |
| ATOM | 3261 | OG | SER | A | 421 | 29.493 | 72.328 | −3.942 | 1.00 | 9.95 | A |
| ATOM | 3262 | C | SER | A | 421 | 32.766 | 73.172 | −5.553 | 1.00 | 8.54 | A |
| ATOM | 3263 | O | SER | A | 421 | 33.612 | 72.276 | −5.532 | 1.00 | 8.31 | A |
| ATOM | 3264 | N | ARG | A | 422 | 33.076 | 74.468 | −5.600 | 1.00 | 8.47 | A |
| ATOM | 3265 | CA | ARG | A | 422 | 34.457 | 74.944 | −5.683 | 1.00 | 8.89 | A |
| ATOM | 3266 | CB | ARG | A | 422 | 35.200 | 74.655 | −4.373 | 1.00 | 9.32 | A |
| ATOM | 3267 | CG | ARG | A | 422 | 35.001 | 75.708 | −3.275 | 1.00 | 8.93 | A |
| ATOM | 3268 | CD | ARG | A | 422 | 33.541 | 75.915 | −2.854 | 1.00 | 8.00 | A |
| ATOM | 3269 | NE | ARG | A | 422 | 33.480 | 76.889 | −1.761 | 1.00 | 8.83 | A |
| ATOM | 3270 | CZ | ARG | A | 422 | 33.533 | 76.580 | −0.468 | 1.00 | 9.12 | A |
| ATOM | 3271 | NH1 | ARG | A | 422 | 33.622 | 75.316 | −0.081 | 1.00 | 10.31 | A |
| ATOM | 3272 | NH2 | ARG | A | 422 | 33.565 | 77.545 | 0.444 | 1.00 | 9.63 | A |
| ATOM | 3273 | C | ARG | A | 422 | 35.174 | 74.285 | −6.865 | 1.00 | 8.93 | A |
| ATOM | 3274 | O | ARG | A | 422 | 36.201 | 73.616 | −6.705 | 1.00 | 8.08 | A |
| ATOM | 3275 | N | PRO | A | 423 | 34.648 | 74.485 | −8.081 | 1.00 | 8.34 | A |
| ATOM | 3276 | CD | PRO | A | 423 | 33.511 | 75.348 | −8.449 | 1.00 | 8.14 | A |
| ATOM | 3277 | CA | PRO | A | 423 | 35.258 | 73.887 | −9.272 | 1.00 | 8.20 | A |
| ATOM | 3278 | CB | PRO | A | 423 | 34.248 | 74.212 | −10.372 | 1.00 | 7.99 | A |
| ATOM | 3279 | CG | PRO | A | 423 | 33.720 | 75.542 | −9.940 | 1.00 | 8.18 | A |
| ATOM | 3280 | C | PRO | A | 423 | 36.679 | 74.342 | −9.606 | 1.00 | 8.42 | A |
| ATOM | 3281 | O | PRO | A | 423 | 37.406 | 73.635 | −10.300 | 1.00 | 7.85 | A |
| ATOM | 3282 | N | TYR | A | 424 | 37.086 | 75.512 | −9.125 | 1.00 | 8.22 | A |
| ATOM | 3283 | CA | TYR | A | 424 | 38.440 | 75.987 | −9.399 | 1.00 | 8.69 | A |
| ATOM | 3284 | CB | TYR | A | 424 | 38.678 | 77.357 | −8.755 | 1.00 | 8.94 | A |
| ATOM | 3285 | CG | TYR | A | 424 | 40.056 | 77.929 | −9.021 | 1.00 | 9.20 | A |
| ATOM | 3286 | CD1 | TYR | A | 424 | 40.304 | 78.697 | −10.156 | 1.00 | 10.09 | A |
| ATOM | 3287 | CE1 | TYR | A | 424 | 41.569 | 79.220 | −10.411 | 1.00 | 11.78 | A |
| ATOM | 3288 | CD2 | TYR | A | 424 | 41.117 | 77.693 | −8.144 | 1.00 | 9.18 | A |
| ATOM | 3289 | CE2 | TYR | A | 424 | 42.388 | 78.211 | −8.392 | 1.00 | 11.36 | A |
| ATOM | 3290 | CZ | TYR | A | 424 | 42.606 | 78.976 | −9.527 | 1.00 | 11.41 | A |
| ATOM | 3291 | OH | TYR | A | 424 | 43.854 | 79.513 | −9.774 | 1.00 | 13.60 | A |
| ATOM | 3292 | C | TYR | A | 424 | 39.446 | 74.997 | −8.818 | 1.00 | 8.12 | A |
| ATOM | 3293 | O | TYR | A | 424 | 40.409 | 74.605 | −9.477 | 1.00 | 8.51 | A |
| ATOM | 3294 | N | HIS | A | 425 | 39.204 | 74.588 | −7.578 | 1.00 | 8.00 | A |
| ATOM | 3295 | CA | HIS | A | 425 | 40.102 | 73.672 | −6.884 | 1.00 | 7.92 | A |
| ATOM | 3296 | CB | HIS | A | 425 | 39.841 | 73.793 | −5.390 | 1.00 | 8.82 | A |
| ATOM | 3297 | CG | HIS | A | 425 | 39.830 | 75.214 | −4.928 | 1.00 | 9.54 | A |
| ATOM | 3298 | CD2 | HIS | A | 425 | 38.817 | 76.107 | −4.842 | 1.00 | 8.63 | A |
| ATOM | 3299 | ND1 | HIS | A | 425 | 40.985 | 75.917 | −4.659 | 1.00 | 11.50 | A |
| ATOM | 3300 | CE1 | HIS | A | 425 | 40.684 | 77.184 | −4.433 | 1.00 | 9.24 | A |
| ATOM | 3301 | NE2 | HIS | A | 425 | 39.376 | 77.326 | −4.540 | 1.00 | 12.26 | A |
| ATOM | 3302 | C | HIS | A | 425 | 39.982 | 72.242 | −7.382 | 1.00 | 7.65 | A |
| ATOM | 3303 | O | HIS | A | 425 | 40.937 | 71.470 | −7.298 | 1.00 | 6.25 | A |
| ATOM | 3304 | N | LYS | A | 426 | 38.809 | 71.891 | −7.900 | 1.00 | 6.92 | A |
| ATOM | 3305 | CA | LYS | A | 426 | 38.601 | 70.566 | −8.474 | 1.00 | 7.10 | A |
| ATOM | 3306 | CB | LYS | A | 426 | 37.130 | 70.386 | −8.863 | 1.00 | 7.64 | A |
| ATOM | 3307 | CG | LYS | A | 426 | 36.233 | 69.957 | −7.707 | 1.00 | 7.33 | A |
| ATOM | 3308 | CD | LYS | A | 426 | 34.758 | 69.974 | −8.108 | 1.00 | 8.18 | A |
| ATOM | 3309 | CE | LYS | A | 426 | 33.893 | 69.112 | −7.180 | 1.00 | 7.20 | A |
| ATOM | 3310 | NZ | LYS | A | 426 | 33.897 | 69.564 | −5.757 | 1.00 | 8.35 | A |
| ATOM | 3311 | C | LYS | A | 426 | 39.500 | 70.461 | −9.711 | 1.00 | 6.80 | A |
| ATOM | 3312 | O | LYS | A | 426 | 40.130 | 69.429 | −9.950 | 1.00 | 7.71 | A |
| ATOM | 3313 | N | ARG | A | 427 | 39.562 | 71.536 | −10.493 | 1.00 | 6.95 | A |
| ATOM | 3314 | CA | ARG | A | 427 | 40.403 | 71.558 | −11.677 | 1.00 | 7.61 | A |
| ATOM | 3315 | CB | ARG | A | 427 | 40.055 | 72.780 | −12.535 | 1.00 | 7.49 | A |
| ATOM | 3316 | CG | ARG | A | 427 | 41.045 | 73.105 | −13.645 | 1.00 | 8.69 | A |
| ATOM | 3317 | CD | ARG | A | 427 | 41.239 | 71.986 | −14.660 | 1.00 | 9.36 | A |
| ATOM | 3318 | NE | ARG | A | 427 | 42.275 | 72.376 | −15.614 | 1.00 | 11.26 | A |
| ATOM | 3319 | CZ | ARG | A | 427 | 43.047 | 71.530 | −16.288 | 1.00 | 11.18 | A |
| ATOM | 3320 | NH1 | ARG | A | 427 | 42.911 | 70.220 | −16.132 | 1.00 | 11.18 | A |
| ATOM | 3321 | NH2 | ARG | A | 427 | 43.985 | 72.004 | −17.101 | 1.00 | 11.85 | A |
| ATOM | 3322 | C | ARG | A | 427 | 41.871 | 71.587 | −11.251 | 1.00 | 7.82 | A |
| ATOM | 3323 | O | ARG | A | 427 | 42.708 | 70.901 | −11.833 | 1.00 | 7.39 | A |
| ATOM | 3324 | N | MSE | A | 428 | 42.179 | 72.370 | −10.220 | 1.00 | 7.89 | A |
| ATOM | 3325 | CA | MSE | A | 428 | 43.550 | 72.457 | −9.729 | 1.00 | 7.92 | A |
| ATOM | 3326 | CB | MSE | A | 428 | 43.613 | 73.415 | −8.542 | 1.00 | 9.64 | A |
| ATOM | 3327 | CG | MSE | A | 428 | 45.022 | 73.778 | −8.109 | 1.00 | 9.04 | A |
| ATOM | 3328 | SE | MSE | A | 428 | 44.978 | 75.197 | −6.818 | 1.00 | 16.90 | A |
| ATOM | 3329 | CE | MSE | A | 428 | 46.844 | 75.667 | −6.794 | 1.00 | 10.50 | A |
| ATOM | 3330 | C | MSE | A | 428 | 44.058 | 71.072 | −9.319 | 1.00 | 7.61 | A |
| ATOM | 3331 | O | MSE | A | 428 | 45.223 | 70.741 | −9.528 | 1.00 | 6.75 | A |
| ATOM | 3332 | N | ASP | A | 429 | 43.173 | 70.264 | −8.742 | 1.00 | 7.36 | A |
| ATOM | 3333 | CA | ASP | A | 429 | 43.532 | 68.915 | −8.323 | 1.00 | 6.76 | A |
| ATOM | 3334 | CB | ASP | A | 429 | 42.302 | 68.199 | −7.764 | 1.00 | 7.80 | A |
| ATOM | 3335 | CG | ASP | A | 429 | 42.567 | 66.742 | −7.454 | 1.00 | 7.25 | A |
| ATOM | 3336 | OD1 | ASP | A | 429 | 42.305 | 65.877 | −8.322 | 1.00 | 7.35 | A |
| ATOM | 3337 | OD2 | ASP | A | 429 | 43.050 | 66.464 | −6.339 | 1.00 | 8.83 | A |
| ATOM | 3338 | C | ASP | A | 429 | 44.110 | 68.101 | −9.477 | 1.00 | 6.82 | A |
| ATOM | 3339 | O | ASP | A | 429 | 45.126 | 67.426 | −9.324 | 1.00 | 6.20 | A |
| ATOM | 3340 | N | ARG | A | 430 | 43.465 | 68.174 | −10.636 | 1.00 | 6.28 | A |
| ATOM | 3341 | CA | ARG | A | 430 | 43.925 | 67.415 | −11.790 | 1.00 | 6.60 | A |
| ATOM | 3342 | CB | ARG | A | 430 | 42.864 | 67.436 | −12.886 | 1.00 | 7.13 | A |
| ATOM | 3343 | CG | ARG | A | 430 | 41.575 | 66.745 | −12.489 | 1.00 | 6.81 | A |
| ATOM | 3344 | CD | ARG | A | 430 | 41.821 | 65.302 | −12.057 | 1.00 | 7.18 | A |
| ATOM | 3345 | NE | ARG | A | 430 | 40.578 | 64.536 | −12.052 | 1.00 | 7.42 | A |
| ATOM | 3346 | CZ | ARG | A | 430 | 39.899 | 64.186 | −10.965 | 1.00 | 7.01 | A |
| ATOM | 3347 | NH1 | ARG | A | 430 | 40.329 | 64.520 | −9.754 | 1.00 | 6.58 | A |
| ATOM | 3348 | NH2 | ARG | A | 430 | 38.770 | 63.504 | −11.092 | 1.00 | 7.28 | A |
| ATOM | 3349 | C | ARG | A | 430 | 45.247 | 67.938 | −12.331 | 1.00 | 6.64 | A |
| ATOM | 3350 | O | ARG | A | 430 | 46.071 | 67.164 | −12.828 | 1.00 | 6.98 | A |
| ATOM | 3351 | N | VAL | A | 431 | 45.448 | 69.246 | −12.248 | 1.00 | 6.93 | A |
| ATOM | 3352 | CA | VAL | A | 431 | 46.693 | 69.838 | −12.717 | 1.00 | 7.00 | A |
| ATOM | 3353 | CB | VAL | A | 431 | 46.613 | 71.381 | −12.697 | 1.00 | 7.25 | A |
| ATOM | 3354 | CG1 | VAL | A | 431 | 47.966 | 71.984 | −13.059 | 1.00 | 7.90 | A |
| ATOM | 3355 | CG2 | VAL | A | 431 | 45.553 | 71.856 | −13.684 | 1.00 | 7.34 | A |
| ATOM | 3356 | C | VAL | A | 431 | 47.835 | 69.361 | −11.814 | 1.00 | 7.33 | A |
| ATOM | 3357 | O | VAL | A | 431 | 48.867 | 68.890 | −12.291 | 1.00 | 6.53 | A |
| ATOM | 3358 | N | LEU | A | 432 | 47.648 | 69.477 | −10.504 | 1.00 | 6.42 | A |
| ATOM | 3359 | CA | LEU | A | 432 | 48.683 | 69.051 | −9.574 | 1.00 | 7.15 | A |
| ATOM | 3360 | CB | LEU | A | 432 | 48.314 | 69.449 | −8.144 | 1.00 | 7.42 | A |
| ATOM | 3361 | CG | LEU | A | 432 | 49.320 | 69.099 | −7.040 | 1.00 | 7.44 | A |
| ATOM | 3362 | CD1 | LEU | A | 432 | 50.706 | 69.655 | −7.368 | 1.00 | 7.97 | A |
| ATOM | 3363 | CD2 | LEU | A | 432 | 48.815 | 69.674 | −5.718 | 1.00 | 7.95 | A |
| ATOM | 3364 | C | LEU | A | 432 | 48.906 | 67.545 | −9.676 | 1.00 | 6.90 | A |
| ATOM | 3365 | O | LEU | A | 432 | 50.031 | 67.073 | −9.538 | 1.00 | 7.14 | A |
| ATOM | 3366 | N | MSE | A | 433 | 47.839 | 66.789 | −9.921 | 1.00 | 6.62 | A |
| ATOM | 3367 | CA | MSE | A | 433 | 47.973 | 65.343 | −10.071 | 1.00 | 6.77 | A |
| ATOM | 3368 | CB | MSE | A | 433 | 46.650 | 64.728 | −10.520 | 1.00 | 7.49 | A |
| ATOM | 3369 | CG | MSE | A | 433 | 46.765 | 63.261 | −10.899 | 1.00 | 7.23 | A |
| ATOM | 3370 | SE | MSE | A | 433 | 45.147 | 62.554 | −11.645 | 1.00 | 14.77 | A |
| ATOM | 3371 | CE | MSE | A | 433 | 45.309 | 63.312 | −13.420 | 1.00 | 11.03 | A |
| ATOM | 3372 | C | MSE | A | 433 | 49.030 | 65.039 | −11.130 | 1.00 | 6.61 | A |
| ATOM | 3373 | O | MSE | A | 433 | 49.915 | 64.198 | −10.933 | 1.00 | 5.99 | A |
| ATOM | 3374 | N | HIS | A | 434 | 48.926 | 65.732 | −12.255 | 1.00 | 5.72 | A |
| ATOM | 3375 | CA | HIS | A | 434 | 49.855 | 65.531 | −13.352 | 1.00 | 6.68 | A |
| ATOM | 3376 | CB | HIS | A | 434 | 49.340 | 66.203 | −14.620 | 1.00 | 7.17 | A |
| ATOM | 3377 | CG | HIS | A | 434 | 50.306 | 66.118 | −15.755 | 1.00 | 6.69 | A |
| ATOM | 3378 | CD2 | HIS | A | 434 | 50.676 | 65.067 | −16.524 | 1.00 | 6.66 | A |
| ATOM | 3379 | ND1 | HIS | A | 434 | 51.107 | 67.172 | −16.134 | 1.00 | 9.42 | A |
| ATOM | 3380 | CE1 | HIS | A | 434 | 51.932 | 66.771 | −17.086 | 1.00 | 6.23 | A |
| ATOM | 3381 | NE2 | HIS | A | 434 | 51.692 | 65.498 | −17.339 | 1.00 | 9.56 | A |
| ATOM | 3382 | C | HIS | A | 434 | 51.259 | 66.038 | −13.054 | 1.00 | 6.95 | A |
| ATOM | 3383 | O | HIS | A | 434 | 52.242 | 65.392 | −13.414 | 1.00 | 6.29 | A |
| ATOM | 3384 | N | TYR | A | 435 | 51.353 | 67.203 | −12.417 | 1.00 | 6.61 | A |
| ATOM | 3385 | CA | TYR | A | 435 | 52.656 | 67.768 | −12.082 | 1.00 | 8.18 | A |
| ATOM | 3386 | CB | TYR | A | 435 | 52.500 | 69.146 | −11.431 | 1.00 | 9.43 | A |
| ATOM | 3387 | CG | TYR | A | 435 | 52.370 | 70.313 | −12.385 | 1.00 | 12.33 | A |
| ATOM | 3388 | CD1 | TYR | A | 435 | 51.432 | 70.309 | −13.417 | 1.00 | 12.80 | A |
| ATOM | 3389 | CE1 | TYR | A | 435 | 51.272 | 71.417 | −14.251 | 1.00 | 13.62 | A |
| ATOM | 3390 | CD2 | TYR | A | 435 | 53.151 | 71.455 | −12.211 | 1.00 | 14.28 | A |
| ATOM | 3391 | CE2 | TYR | A | 435 | 53.001 | 72.562 | −13.034 | 1.00 | 15.81 | A |
| ATOM | 3392 | CZ | TYR | A | 435 | 52.059 | 72.541 | −14.050 | 1.00 | 15.15 | A |
| ATOM | 3393 | OH | TYR | A | 435 | 51.896 | 73.658 | −14.841 | 1.00 | 17.94 | A |
| ATOM | 3394 | C | TYR | A | 435 | 53.414 | 66.847 | −11.128 | 1.00 | 6.75 | A |
| ATOM | 3395 | O | TYR | A | 435 | 54.636 | 66.719 | −11.226 | 1.00 | 7.81 | A |
| ATOM | 3396 | N | VAL | A | 436 | 52.700 | 66.218 | −10.195 | 1.00 | 5.90 | A |
| ATOM | 3397 | CA | VAL | A | 436 | 53.355 | 65.305 | −9.263 | 1.00 | 6.76 | A |
| ATOM | 3398 | CB | VAL | A | 436 | 52.382 | 64.850 | −8.153 | 1.00 | 6.54 | A |
| ATOM | 3399 | CG1 | VAL | A | 436 | 52.963 | 63.665 | −7.384 | 1.00 | 7.70 | A |
| ATOM | 3400 | CG2 | VAL | A | 436 | 52.132 | 66.009 | −7.200 | 1.00 | 6.54 | A |
| ATOM | 3401 | C | VAL | A | 436 | 53.892 | 64.101 | −10.034 | 1.00 | 6.75 | A |
| ATOM | 3402 | O | VAL | A | 436 | 55.041 | 63.698 | −9.857 | 1.00 | 6.38 | A |
| ATOM | 3403 | N | ARG | A | 437 | 53.069 | 63.532 | −10.905 | 1.00 | 5.91 | A |
| ATOM | 3404 | CA | ARG | A | 437 | 53.518 | 62.393 | −11.693 | 1.00 | 6.53 | A |
| ATOM | 3405 | CB | ARG | A | 437 | 52.406 | 61.902 | −12.621 | 1.00 | 6.39 | A |
| ATOM | 3406 | CG | ARG | A | 437 | 52.904 | 60.916 | −13.674 | 1.00 | 7.38 | A |
| ATOM | 3407 | CD | ARG | A | 437 | 51.779 | 60.403 | −14.552 | 1.00 | 7.95 | A |
| ATOM | 3408 | NE | ARG | A | 437 | 52.287 | 59.711 | −15.735 | 1.00 | 6.99 | A |
| ATOM | 3409 | CZ | ARG | A | 437 | 51.550 | 58.897 | −16.486 | 1.00 | 7.60 | A |
| ATOM | 3410 | NH1 | ARG | A | 437 | 50.282 | 58.670 | −16.170 | 1.00 | 8.52 | A |
| ATOM | 3411 | NH2 | ARG | A | 437 | 52.075 | 58.321 | −17.559 | 1.00 | 7.42 | A |
| ATOM | 3412 | C | ARG | A | 437 | 54.743 | 62.754 | −12.528 | 1.00 | 6.68 | A |
| ATOM | 3413 | O | ARG | A | 437 | 55.725 | 62.009 | −12.558 | 1.00 | 6.83 | A |
| ATOM | 3414 | N | ALA | A | 438 | 54.682 | 63.892 | −13.217 | 1.00 | 6.53 | A |
| ATOM | 3415 | CA | ALA | A | 438 | 55.790 | 64.321 | −14.066 | 1.00 | 7.02 | A |
| ATOM | 3416 | CB | ALA | A | 438 | 55.385 | 65.550 | −14.887 | 1.00 | 7.20 | A |
| ATOM | 3417 | C | ALA | A | 438 | 57.061 | 64.613 | −13.269 | 1.00 | 6.87 | A |
| ATOM | 3418 | O | ALA | A | 438 | 58.165 | 64.269 | −13.702 | 1.00 | 8.03 | A |
| ATOM | 3419 | N | ALA | A | 439 | 56.909 | 65.247 | −12.110 | 1.00 | 7.12 | A |
| ATOM | 3420 | CA | ALA | A | 439 | 58.061 | 65.569 | −11.276 | 1.00 | 6.67 | A |
| ATOM | 3421 | CB | ALA | A | 439 | 57.637 | 66.475 | −10.123 | 1.00 | 7.46 | A |
| ATOM | 3422 | C | ALA | A | 439 | 58.708 | 64.289 | −10.741 | 1.00 | 7.35 | A |
| ATOM | 3423 | O | ALA | A | 439 | 59.930 | 64.141 | −10.770 | 1.00 | 6.65 | A |
| ATOM | 3424 | N | GLU | A | 440 | 57.897 | 63.360 | −10.248 | 1.00 | 6.24 | A |
| ATOM | 3425 | CA | GLU | A | 440 | 58.458 | 62.116 | −9.739 | 1.00 | 7.04 | A |
| ATOM | 3426 | CB | GLU | A | 440 | 57.382 | 61.279 | −9.041 | 1.00 | 7.73 | A |
| ATOM | 3427 | CG | GLU | A | 440 | 56.853 | 61.911 | −7.762 | 1.00 | 8.18 | A |
| ATOM | 3428 | CD | GLU | A | 440 | 56.186 | 60.904 | −6.843 | 1.00 | 10.22 | A |
| ATOM | 3429 | OE1 | GLU | A | 440 | 55.089 | 60.406 | −7.173 | 1.00 | 10.17 | A |
| ATOM | 3430 | OE2 | GLU | A | 440 | 56.773 | 60.603 | −5.787 | 1.00 | 10.50 | A |
| ATOM | 3431 | C | GLU | A | 440 | 59.112 | 61.292 | −10.851 | 1.00 | 6.93 | A |
| ATOM | 3432 | O | GLU | A | 440 | 60.152 | 60.676 | −10.639 | 1.00 | 7.92 | A |
| ATOM | 3433 | N | MSE | A | 441 | 58.517 | 61.285 | −12.040 | 1.00 | 6.93 | A |
| ATOM | 3434 | CA | MSE | A | 441 | 59.084 | 60.513 | −13.141 | 1.00 | 7.41 | A |
| ATOM | 3435 | CB | MSE | A | 441 | 58.068 | 60.378 | −14.277 | 1.00 | 8.35 | A |
| ATOM | 3436 | CG | MSE | A | 441 | 58.580 | 59.571 | −15.465 | 1.00 | 8.46 | A |
| ATOM | 3437 | SE | MSE | A | 441 | 57.255 | 59.396 | −16.865 | 1.00 | 13.95 | A |
| ATOM | 3438 | CE | MSE | A | 441 | 56.081 | 58.134 | −15.968 | 1.00 | 10.73 | A |
| ATOM | 3439 | C | MSE | A | 441 | 60.377 | 61.120 | −13.677 | 1.00 | 7.44 | A |
| ATOM | 3440 | O | MSE | A | 441 | 61.381 | 60.423 | −13.818 | 1.00 | 8.04 | A |
| ATOM | 3441 | N | LEU | A | 442 | 60.354 | 62.416 | −13.979 | 1.00 | 7.67 | A |
| ATOM | 3442 | CA | LEU | A | 442 | 61.539 | 63.085 | −14.509 | 1.00 | 8.05 | A |
| ATOM | 3443 | CB | LEU | A | 442 | 61.233 | 64.553 | −14.813 | 1.00 | 8.28 | A |
| ATOM | 3444 | CG | LEU | A | 442 | 60.557 | 64.823 | −16.161 | 1.00 | 8.01 | A |
| ATOM | 3445 | CD1 | LEU | A | 442 | 59.999 | 66.236 | −16.193 | 1.00 | 9.63 | A |
| ATOM | 3446 | CD2 | LEU | A | 442 | 61.564 | 64.612 | −17.283 | 1.00 | 10.08 | A |
| ATOM | 3447 | C | LEU | A | 442 | 62.750 | 62.999 | −13.592 | 1.00 | 8.72 | A |
| ATOM | 3448 | O | LEU | A | 442 | 63.881 | 62.855 | −14.060 | 1.00 | 8.81 | A |
| ATOM | 3449 | N | SER | A | 443 | 62.523 | 63.074 | −12.284 | 1.00 | 8.42 | A |
| ATOM | 3450 | CA | SER | A | 443 | 63.635 | 63.020 | −11.346 | 1.00 | 8.63 | A |
| ATOM | 3451 | CE | SER | A | 443 | 63.306 | 63.818 | −10.077 | 1.00 | 8.25 | A |
| ATOM | 3452 | OG | SER | A | 443 | 62.192 | 63.273 | −9.387 | 1.00 | 8.38 | A |
| ATOM | 3453 | C | SER | A | 443 | 64.038 | 61.599 | −10.975 | 1.00 | 9.17 | A |
| ATOM | 3454 | O | SER | A | 443 | 65.105 | 61.388 | −10.399 | 1.00 | 10.14 | A |
| ATOM | 3455 | N | ALA | A | 444 | 63.201 | 60.628 | −11.324 | 1.00 | 8.08 | A |
| ATOM | 3456 | CA | ALA | A | 444 | 63.488 | 59.231 | −11.004 | 1.00 | 8.59 | A |
| ATOM | 3457 | CB | ALA | A | 444 | 62.237 | 58.383 | −11.202 | 1.00 | 9.58 | A |
| ATOM | 3458 | C | ALA | A | 444 | 64.629 | 58.652 | −11.833 | 1.00 | 9.05 | A |
| ATOM | 3459 | O | ALA | A | 444 | 65.268 | 57.689 | −11.416 | 1.00 | 9.90 | A |
| ATOM | 3460 | N | TRP | A | 445 | 64.886 | 59.229 | −13.002 | 1.00 | 9.47 | A |
| ATOM | 3461 | CA | TRP | A | 445 | 65.939 | 58.712 | −13.869 | 1.00 | 10.27 | A |
| ATOM | 3462 | CB | TRP | A | 445 | 65.943 | 59.452 | −15.210 | 1.00 | 9.48 | A |
| ATOM | 3463 | CG | TRP | A | 445 | 64.665 | 59.287 | −15.975 | 1.00 | 8.72 | A |
| ATOM | 3464 | CD2 | TRP | A | 445 | 64.211 | 58.103 | −16.648 | 1.00 | 8.20 | A |
| ATOM | 3465 | CE2 | TRP | A | 445 | 62.926 | 58.384 | −17.163 | 1.00 | 7.89 | A |
| ATOM | 3466 | CE3 | TRP | A | 445 | 64.763 | 56.833 | −16.862 | 1.00 | 9.03 | A |
| ATOM | 3467 | CD1 | TRP | A | 445 | 63.670 | 60.209 | −16.112 | 1.00 | 9.25 | A |
| ATOM | 3468 | NE1 | TRP | A | 445 | 62.623 | 59.675 | −16.821 | 1.00 | 8.67 | A |
| ATOM | 3469 | CZ2 | TRP | A | 445 | 62.181 | 57.441 | −17.881 | 1.00 | 7.85 | A |
| ATOM | 3470 | CZ3 | TRP | A | 445 | 64.024 | 55.893 | −17.575 | 1.00 | 9.31 | A |
| ATOM | 3471 | CH2 | TRP | A | 445 | 62.744 | 56.204 | −18.076 | 1.00 | 8.54 | A |
| ATOM | 3472 | C | TRP | A | 445 | 67.329 | 58.772 | −13.251 | 1.00 | 10.91 | A |
| ATOM | 3473 | O | TRP | A | 445 | 68.195 | 57.968 | −13.594 | 1.00 | 12.14 | A |
| ATOM | 3474 | N | HIS | A | 446 | 67.541 | 59.724 | −12.348 | 1.00 | 11.75 | A |
| ATOM | 3475 | CA | HIS | A | 446 | 68.836 | 59.870 | −11.691 | 1.00 | 12.74 | A |
| ATOM | 3476 | CB | HIS | A | 446 | 69.470 | 61.232 | −11.985 | 1.00 | 14.40 | A |
| ATOM | 3477 | CG | HIS | A | 446 | 69.853 | 61.443 | −13.413 | 1.00 | 15.98 | A |
| ATOM | 3478 | CD2 | HIS | A | 446 | 71.011 | 61.196 | −14.070 | 1.00 | 17.49 | A |
| ATOM | 3479 | ND1 | HIS | A | 446 | 69.000 | 62.008 | −14.336 | 1.00 | 16.71 | A |
| ATOM | 3480 | CE1 | HIS | A | 446 | 69.618 | 62.103 | −15.500 | 1.00 | 17.82 | A |
| ATOM | 3481 | NE2 | HIS | A | 446 | 70.838 | 61.618 | −15.365 | 1.00 | 17.18 | A |
| ATOM | 3482 | C | HIS | A | 446 | 68.734 | 59.775 | −10.181 | 1.00 | 12.44 | A |
| ATOM | 3483 | O | HIS | A | 446 | 67.658 | 59.891 | −9.595 | 1.00 | 12.19 | A |
| ATOM | 3484 | N | SER | A | 447 | 69.887 | 59.573 | −9.559 | 1.00 | 13.41 | A |
| ATOM | 3485 | CA | SER | A | 447 | 69.984 | 59.553 | −8.115 | 1.00 | 13.93 | A |
| ATOM | 3486 | CB | SER | A | 447 | 71.047 | 58.549 | −7.667 | 1.00 | 15.77 | A |
| ATOM | 3487 | OG | SER | A | 447 | 71.107 | 58.470 | −6.255 | 1.00 | 19.30 | A |
| ATOM | 3488 | C | SER | A | 447 | 70.452 | 60.989 | −7.864 | 1.00 | 14.00 | A |
| ATOM | 3489 | O | SER | A | 447 | 71.316 | 61.492 | −8.586 | 1.00 | 14.59 | A |
| ATOM | 3490 | N | TRP | A | 448 | 69.874 | 61.665 | −6.878 | 1.00 | 13.08 | A |
| ATOM | 3491 | CA | TRP | A | 448 | 70.259 | 63.045 | −6.614 | 1.00 | 13.81 | A |
| ATOM | 3492 | CB | TRP | A | 448 | 69.037 | 63.961 | −6.688 | 1.00 | 13.00 | A |
| ATOM | 3493 | CG | TRP | A | 448 | 68.381 | 63.979 | −8.032 | 1.00 | 11.48 | A |
| ATOM | 3494 | CD2 | TRP | A | 448 | 68.523 | 64.987 | −9.035 | 1.00 | 10.99 | A |
| ATOM | 3495 | CE2 | TRP | A | 448 | 67.738 | 64.590 | −10.140 | 1.00 | 10.00 | A |
| ATOM | 3496 | CE3 | TRP | A | 448 | 69.238 | 66.192 | −9.108 | 1.00 | 11.04 | A |
| ATOM | 3497 | CD1 | TRP | A | 448 | 67.541 | 63.035 | −8.551 | 1.00 | 11.29 | A |
| ATOM | 3498 | NE1 | TRP | A | 448 | 67.150 | 63.395 | −9.818 | 1.00 | 10.64 | A |
| ATOM | 3499 | CZ2 | TRP | A | 448 | 67.646 | 65.354 | −11.306 | 1.00 | 11.20 | A |
| ATOM | 3500 | CZ3 | TRP | A | 448 | 69.147 | 66.952 | −10.270 | 1.00 | 10.82 | A |
| ATOM | 3501 | CH2 | TRP | A | 448 | 68.355 | 66.527 | −11.353 | 1.00 | 11.03 | A |
| ATOM | 3502 | C | TRP | A | 448 | 70.945 | 63.261 | −5.278 | 1.00 | 15.52 | A |
| ATOM | 3503 | O | TRP | A | 448 | 70.609 | 62.621 | −4.283 | 1.00 | 15.44 | A |
| ATOM | 3504 | N | ASP | A | 449 | 71.905 | 64.180 | −5.270 | 1.00 | 17.52 | A |
| ATOM | 3505 | CA | ASP | A | 449 | 72.627 | 64.520 | −4.055 | 1.00 | 19.65 | A |
| ATOM | 3506 | CB | ASP | A | 449 | 73.780 | 65.473 | −4.380 | 1.00 | 22.50 | A |
| ATOM | 3507 | CG | ASP | A | 449 | 74.655 | 65.764 | −3.177 | 1.00 | 25.00 | A |
| ATOM | 3508 | OD1 | ASP | A | 449 | 74.191 | 66.458 | −2.248 | 1.00 | 26,37 | A |
| ATOM | 3509 | OD2 | ASP | A | 449 | 75.811 | 65.290 | −3.161 | 1.00 | 27.45 | A |
| ATOM | 3510 | C | ASP | A | 449 | 71.627 | 65.202 | −3.128 | 1.00 | 19.63 | A |
| ATOM | 3511 | O | ASP | A | 449 | 70.729 | 65.909 | −3.589 | 1.00 | 19.29 | A |
| ATOM | 3512 | N | GLY | A | 450 | 71.780 | 64.985 | −1.826 | 1.00 | 20.01 | A |
| ATOM | 3513 | CA | GLY | A | 450 | 70.870 | 65.581 | −0.864 | 1.00 | 20.00 | A |
| ATOM | 3514 | C | GLY | A | 450 | 70.735 | 67.089 | −0.962 | 1.00 | 20.23 | A |
| ATOM | 3515 | O | GLY | A | 450 | 69.690 | 67.646 | −0.616 | 1.00 | 19.88 | A |
| ATOM | 3516 | N | MET | A | 451 | 71.786 | 67.755 | −1.432 | 1.00 | 20.10 | A |
| ATOM | 3517 | CA | MET | A | 451 | 71.768 | 69.209 | −1.561 | 1.00 | 20.63 | A |
| ATOM | 3518 | CB | MET | A | 451 | 73.166 | 69.735 | −1.896 | 1.00 | 23.63 | A |
| ATOM | 3519 | CG | MET | A | 451 | 74.203 | 69.506 | −0.815 | 1.00 | 27.14 | A |
| ATOM | 3520 | SD | MET | A | 451 | 75.744 | 70.366 | −1.188 | 1.00 | 32.31 | A |
| ATOM | 3521 | CE | MET | A | 451 | 76.487 | 69.235 | −2.362 | 1.00 | 29.66 | A |
| ATOM | 3522 | C | MET | A | 451 | 70.787 | 69.704 | −2.616 | 1.00 | 18.82 | A |
| ATOM | 3523 | O | MET | A | 451 | 70.419 | 70.877 | −2.622 | 1.00 | 18.97 | A |
| ATOM | 3524 | N | ALA | A | 452 | 70.373 | 68.814 | −3.512 | 1.00 | 17.14 | A |
| ATOM | 3525 | CA | ALA | A | 452 | 69.433 | 69.181 | −4.564 | 1.00 | 16.09 | A |
| ATOM | 3526 | CB | ALA | A | 452 | 69.469 | 68.149 | −5.682 | 1.00 | 15.55 | A |
| ATOM | 3527 | C | ALA | A | 452 | 68.019 | 69.307 | −4.006 | 1.00 | 15.58 | A |
| ATOM | 3528 | O | ALA | A | 452 | 67.134 | 69.862 | −4.655 | 1.00 | 15.31 | A |
| ATOM | 3529 | N | ARG | A | 453 | 67.816 | 68.780 | −2.802 | 1.00 | 14.93 | A |
| ATOM | 3530 | CA | ARG | A | 453 | 66.520 | 68.844 | −2.133 | 1.00 | 15.12 | A |
| ATOM | 3531 | CB | ARG | A | 453 | 66.209 | 70.296 | −1.757 | 1.00 | 16.65 | A |
| ATOM | 3532 | CG | ARG | A | 453 | 67.260 | 70.927 | −0.853 | 1.00 | 19.15 | A |
| ATOM | 3533 | CD | ARG | A | 453 | 67.014 | 72.415 | −0.653 | 1.00 | 21.70 | A |
| ATOM | 3534 | NE | ARG | A | 453 | 65.762 | 72.680 | 0.047 | 1.00 | 23.84 | A |
| ATOM | 3535 | CZ | ARG | A | 453 | 65.266 | 73.896 | 0.255 | 1.00 | 25.33 | A |
| ATOM | 3536 | NH1 | ARG | A | 453 | 65.917 | 74.967 | −0.187 | 1.00 | 25.98 | A |
| ATOM | 3537 | NH2 | ARG | A | 453 | 64.120 | 74.043 | 0.905 | 1.00 | 25.91 | A |
| ATOM | 3538 | C | ARG | A | 453 | 65.376 | 68.269 | −2.967 | 1.00 | 13.88 | A |
| ATOM | 3539 | O | ARG | A | 453 | 64.232 | 68.701 | −2.843 | 1.00 | 14.25 | A |
| ATOM | 3540 | N | ILE | A | 454 | 65.683 | 67.289 | −3.810 | 1.00 | 11.74 | A |
| ATOM | 3541 | CA | ILE | A | 454 | 64.666 | 66.666 | −4.648 | 1.00 | 11.41 | A |
| ATOM | 3542 | CB | ILE | A | 454 | 65.310 | 65.691 | −5.662 | 1.00 | 11.24 | A |
| ATOM | 3543 | CG2 | ILE | A | 454 | 64.225 | 64.960 | −6.447 | 1.00 | 12.28 | A |
| ATOM | 3544 | CG1 | ILE | A | 454 | 66.260 | 66.456 | −6.594 | 1.00 | 12.69 | A |
| ATOM | 3545 | CD1 | ILE | A | 454 | 65.605 | 67.562 | −7.396 | 1.00 | 13.45 | A |
| ATOM | 3546 | C | ILE | A | 454 | 63.633 | 65.910 | −3.804 | 1.00 | 11.43 | A |
| ATOM | 3547 | O | ILE | A | 454 | 62.429 | 66.142 | −3.936 | 1.00 | 10.95 | A |
| ATOM | 3548 | N | GLU | A | 455 | 64.100 | 65.013 | −2.939 | 1.00 | 11.35 | A |
| ATOM | 3549 | CA | GLU | A | 455 | 63.196 | 64.234 | −2.092 | 1.00 | 11.75 | A |
| ATOM | 3550 | CB | GLU | A | 455 | 63.981 | 63.274 | −1.188 | 1.00 | 12.29 | A |
| ATOM | 3551 | CG | GLU | A | 455 | 64.595 | 62.071 | −1.896 | 1.00 | 12.76 | A |
| ATOM | 3552 | CD | GLU | A | 455 | 65.938 | 62.375 | −2.539 | 1.00 | 13.14 | A |
| ATOM | 3553 | OE1 | GLU | A | 455 | 66.381 | 63.541 | −2.473 | 1.00 | 14.41 | A |
| ATOM | 3554 | OE2 | GLU | A | 455 | 66.544 | 61.441 | −3.108 | 1.00 | 12.50 | A |
| ATOM | 3555 | C | GLU | A | 455 | 62.337 | 65.153 | −1.230 | 1.00 | 11.49 | A |
| ATOM | 3556 | O | GLU | A | 455 | 61.145 | 64.907 | −1.025 | 1.00 | 11.26 | A |
| ATOM | 3557 | N | GLU | A | 456 | 62.948 | 66.218 | −0.729 | 1.00 | 12.01 | A |
| ATOM | 3558 | CA | GLU | A | 456 | 62.249 | 67.183 | 0.107 | 1.00 | 12.19 | A |
| ATOM | 3559 | CB | GLU | A | 456 | 63.216 | 68.281 | 0.545 | 1.00 | 14.58 | A |
| ATOM | 3560 | CG | GLU | A | 456 | 62.624 | 69.300 | 1.494 | 1.00 | 17.19 | A |
| ATOM | 3561 | CD | GLU | A | 456 | 63.514 | 70.515 | 1.653 | 1.00 | 18.91 | A |
| ATOM | 3562 | OE1 | GLU | A | 456 | 64.749 | 70.360 | 1.554 | 1.00 | 20.97 | A |
| ATOM | 3563 | OE2 | GLU | A | 456 | 62.982 | 71.621 | 1.887 | 1.00 | 20.95 | A |
| ATOM | 3564 | C | GLU | A | 456 | 61.072 | 67.817 | −0.635 | 1.00 | 11.41 | A |
| ATOM | 3565 | O | GLU | A | 456 | 59.950 | 67.859 | −0.126 | 1.00 | 11.43 | A |
| ATOM | 3566 | N | ARG | A | 457 | 61.332 | 68.321 | −1.836 | 1.00 | 11.00 | A |
| ATOM | 3567 | CA | ARG | A | 457 | 60.286 | 68.964 | −2.622 | 1.00 | 10.38 | A |
| ATOM | 3568 | CB | ARG | A | 457 | 60.898 | 69.660 | −3.847 | 1.00 | 11.74 | A |
| ATOM | 3569 | CG | ARG | A | 457 | 61.302 | 71.116 | −3.608 | 1.00 | 14.55 | A |
| ATOM | 3570 | CD | ARG | A | 457 | 62.235 | 71.277 | −2.416 | 1.00 | 17.43 | A |
| ATOM | 3571 | NE | ARG | A | 457 | 62.539 | 72.681 | −2.130 | 1.00 | 19.26 | A |
| ATOM | 3572 | CZ | ARG | A | 457 | 63.355 | 73.442 | −2.855 | 1.00 | 21.10 | A |
| ATOM | 3573 | NH1 | ARG | A | 457 | 63.963 | 72.944 | −3.924 | 1.00 | 21.18 | A |
| ATOM | 3574 | NH2 | ARG | A | 457 | 63.566 | 74.706 | −2.507 | 1.00 | 21.77 | A |
| ATOM | 3575 | C | ARG | A | 457 | 59.191 | 67.995 | −3.054 | 1.00 | 9.31 | A |
| ATOM | 3576 | O | ARG | A | 457 | 58.013 | 68.348 | −3.048 | 1.00 | 8.43 | A |
| ATOM | 3577 | N | LEU | A | 458 | 59.575 | 66.776 | −3.421 | 1.00 | 8.92 | A |
| ATOM | 3578 | CA | LEU | A | 458 | 58.594 | 65.783 | −3.844 | 1.00 | 9.14 | A |
| ATOM | 3579 | CB | LEU | A | 458 | 59.293 | 64.572 | −4.465 | 1.00 | 8.78 | A |
| ATOM | 3580 | CG | LEU | A | 458 | 59.992 | 64.878 | −5.799 | 1.00 | 8.90 | A |
| ATOM | 3581 | CD1 | LEU | A | 458 | 60.677 | 63.624 | −6.313 | 1.00 | 9.97 | A |
| ATOM | 3582 | CD2 | LEU | A | 458 | 58.970 | 65.395 | −6.825 | 1.00 | 9.94 | A |
| ATOM | 3583 | C | LEU | A | 458 | 57.706 | 65.350 | −2.685 | 1.00 | 9.64 | A |
| ATOM | 3584 | O | LEU | A | 458 | 56.511 | 65.124 | −2.872 | 1.00 | 10.33 | A |
| ATOM | 3585 | N | GLU | A | 459 | 58.277 | 65.243 | −1.487 | 1.00 | 9.21 | A |
| ATOM | 3586 | CA | GLU | A | 459 | 57.481 | 64.852 | −0.328 | 1.00 | 9.96 | A |
| ATOM | 3587 | CB | GLU | A | 459 | 58.372 | 64.654 | 0.902 | 1.00 | 11.30 | A |
| ATOM | 3588 | CG | GLU | A | 459 | 57.603 | 64.225 | 2.146 | 1.00 | 13.61 | A |
| ATOM | 3589 | CD | GLU | A | 459 | 58.497 | 63.617 | 3.213 | 1.00 | 15.23 | A |
| ATOM | 3590 | OE1 | GLU | A | 459 | 59.217 | 64.371 | 3.897 | 1.00 | 18.32 | A |
| ATOM | 3591 | OE2 | GLU | A | 459 | 58.482 | 62.377 | 3.358 | 1.00 | 17.20 | A |
| ATOM | 3592 | C | GLU | A | 459 | 56.445 | 65.940 | −0.059 | 1.00 | 9.87 | A |
| ATOM | 3593 | O | GLU | A | 459 | 55.279 | 65.653 | 0.205 | 1.00 | 8.53 | A |
| ATOM | 3594 | N | GLN | A | 460 | 56.874 | 67.197 | −0.137 | 1.00 | 8.94 | A |
| ATOM | 3595 | CA | GLN | A | 460 | 55.962 | 68.312 | 0.083 | 1.00 | 9.46 | A |
| ATOM | 3596 | CB | GLN | A | 460 | 56.706 | 69.640 | −0.067 | 1.00 | 11.33 | A |
| ATOM | 3597 | CG | GLN | A | 460 | 55.828 | 70.862 | 0.130 | 1.00 | 14.48 | A |
| ATOM | 3598 | CD | GLN | A | 460 | 56.541 | 72.153 | −0.221 | 1.00 | 16.73 | A |
| ATOM | 3599 | OE1 | GLN | A | 460 | 56.024 | 73.242 | 0.020 | 1.00 | 20.84 | A |
| ATOM | 3600 | NE2 | GLN | A | 460 | 57.729 | 72.036 | −0.803 | 1.00 | 18.45 | A |
| ATOM | 3601 | C | GLN | A | 460 | 54.823 | 68.246 | −0.937 | 1.00 | 9.40 | A |
| ATOM | 3602 | O | GLN | A | 460 | 53.651 | 68.330 | −0.580 | 1.00 | 8.87 | A |
| ATOM | 3603 | N | ALA | A | 461 | 55.169 | 68.081 | −2.210 | 1.00 | 8.69 | A |
| ATOM | 3604 | CA | ALA | A | 461 | 54.146 | 68.023 | −3.247 | 1.00 | 8.76 | A |
| ATOM | 3605 | CB | ALA | A | 461 | 54.796 | 67.910 | −4.622 | 1.00 | 8.72 | A |
| ATOM | 3606 | C | ALA | A | 461 | 53.160 | 66.877 | −3.034 | 1.00 | 8.44 | A |
| ATOM | 3607 | O | ALA | A | 461 | 51.947 | 67.073 | −3.113 | 1.00 | 8.84 | A |
| ATOM | 3608 | N | ARG | A | 462 | 53.678 | 65.684 | −2.763 | 1.00 | 7.93 | A |
| ATOM | 3609 | CA | ARG | A | 462 | 52.812 | 64.529 | −2.548 | 1.00 | 7.87 | A |
| ATOM | 3610 | CB | ARG | A | 462 | 53.629 | 63.255 | −2.292 | 1.00 | 7.57 | A |
| ATOM | 3611 | CG | ARG | A | 462 | 54.346 | 62.679 | −3.515 | 1.00 | 7.40 | A |
| ATOM | 3612 | CD | ARG | A | 462 | 54.815 | 61.252 | −3.233 | 1.00 | 7.74 | A |
| ATOM | 3613 | NE | ARG | A | 462 | 55.726 | 61.190 | −2.089 | 1.00 | 7.74 | A |
| ATOM | 3614 | CZ | ARG | A | 462 | 57.043 | 61.352 | −2.169 | 1.00 | 8.73 | A |
| ATOM | 3615 | NH1 | ARG | A | 462 | 57.621 | 61.578 | −3.344 | 1.00 | 8.89 | A |
| ATOM | 3616 | NH2 | ARG | A | 462 | 57.787 | 61.303 | −1.070 | 1.00 | 9.62 | A |
| ATOM | 3617 | C | ARG | A | 462 | 51.882 | 64.747 | −1.365 | 1.00 | 8.16 | A |
| ATOM | 3618 | O | ARG | A | 462 | 50.707 | 64.389 | −1.416 | 1.00 | 7.32 | A |
| ATOM | 3619 | N | ARG | A | 463 | 52.399 | 65.344 | −0.298 | 1.00 | 8.35 | A |
| ATOM | 3620 | CA | ARG | A | 463 | 51.576 | 65.546 | 0.884 | 1.00 | 9.21 | A |
| ATOM | 3621 | CB | ARG | A | 463 | 52.473 | 65.823 | 2.093 | 1.00 | 9.46 | A |
| ATOM | 3622 | CG | ARG | A | 463 | 53.219 | 64.564 | 2.500 | 1.00 | 9.86 | A |
| ATOM | 3623 | CD | ARG | A | 463 | 54.152 | 64.737 | 3.681 | 1.00 | 11.93 | A |
| ATOM | 3624 | NE | ARG | A | 463 | 54.673 | 63.433 | 4.075 | 1.00 | 13.77 | A |
| ATOM | 3625 | CZ | ARG | A | 463 | 55.453 | 63.216 | 5.128 | 1.00 | 14.71 | A |
| ATOM | 3626 | NH1 | ARG | A | 463 | 55.815 | 64.225 | 5.906 | 1.00 | 15.88 | A |
| ATOM | 3627 | NH2 | ARG | A | 463 | 55.857 | 61.982 | 5.405 | 1.00 | 15.92 | A |
| ATOM | 3628 | C | ARG | A | 463 | 50.485 | 66.599 | 0.742 | 1.00 | 9.13 | A |
| ATOM | 3629 | O | ARG | A | 463 | 49.386 | 66.420 | 1.266 | 1.00 | 8.63 | A |
| ATOM | 3630 | N | GLU | A | 464 | 50.760 | 67.681 | 0.020 | 1.00 | 8.86 | A |
| ATOM | 3631 | CA | GLU | A | 464 | 49.743 | 68.712 | −0.149 | 1.00 | 8.79 | A |
| ATOM | 3632 | CB | GLU | A | 464 | 50.359 | 69.995 | −0.719 | 1.00 | 10.23 | A |
| ATOM | 3633 | CG | GLU | A | 464 | 51.526 | 70.535 | 0.118 | 1.00 | 12.65 | A |
| ATOM | 3634 | CD | GLU | A | 464 | 51.115 | 71.035 | 1.501 | 1.00 | 15.53 | A |
| ATOM | 3635 | OE1 | GLU | A | 464 | 50.127 | 70.527 | 2.075 | 1.00 | 15.74 | A |
| ATOM | 3636 | OE2 | GLU | A | 464 | 51.802 | 71.936 | 2.027 | 1.00 | 18.42 | A |
| ATOM | 3637 | C | GLU | A | 464 | 48.638 | 68.193 | −1.064 | 1.00 | 8.27 | A |
| ATOM | 3638 | O | GLU | A | 464 | 47.459 | 68.460 | −0.830 | 1.00 | 8.83 | A |
| ATOM | 3639 | N | LEU | A | 465 | 49.012 | 67.449 | −2.102 | 1.00 | 8.19 | A |
| ATOM | 3640 | CA | LEU | A | 465 | 48.008 | 66.887 | −3.000 | 1.00 | 7.76 | A |
| ATOM | 3641 | CB | LEU | A | 465 | 48.666 | 66.269 | −4.240 | 1.00 | 7.54 | A |
| ATOM | 3642 | CG | LEU | A | 465 | 47.706 | 65.594 | −5.231 | 1.00 | 7.30 | A |
| ATOM | 3643 | CD1 | LEU | A | 465 | 46.652 | 66.591 | −5.710 | 1.00 | 8.29 | A |
| ATOM | 3644 | CD2 | LEU | A | 465 | 48.502 | 65.043 | −6.405 | 1.00 | 8.63 | A |
| ATOM | 3645 | C | LEU | A | 465 | 47.219 | 65.817 | −2.241 | 1.00 | 7.24 | A |
| ATOM | 3646 | O | LEU | A | 465 | 45.995 | 65.720 | −2.368 | 1.00 | 7.02 | A |
| ATOM | 3647 | N | SER | A | 466 | 47.920 | 65.014 | −1.444 | 1.00 | 6.60 | A |
| ATOM | 3648 | CA | SER | A | 466 | 47.258 | 63.972 | −0.670 | 1.00 | 7.04 | A |
| ATOM | 3649 | CB | SER | A | 466 | 48.284 | 63.145 | 0.106 | 1.00 | 7.46 | A |
| ATOM | 3650 | OG | SER | A | 466 | 49.039 | 62.325 | −0.766 | 1.00 | 8.02 | A |
| ATOM | 3651 | C | SER | A | 466 | 46.249 | 64.567 | 0.303 | 1.00 | 6.94 | A |
| ATOM | 3652 | O | SER | A | 466 | 45.140 | 64.053 | 0.443 | 1.00 | 6.64 | A |
| ATOM | 3653 | N | LEU | A | 467 | 46.638 | 65.649 | 0.969 | 1.00 | 6.68 | A |
| ATOM | 3654 | CA | LEU | A | 467 | 45.759 | 66.306 | 1.922 | 1.00 | 7.53 | A |
| ATOM | 3655 | CB | LEU | A | 467 | 46.462 | 67.522 | 2.532 | 1.00 | 7.48 | A |
| ATOM | 3656 | CG | LEU | A | 467 | 45.653 | 68.259 | 3.601 | 1.00 | 9.74 | A |
| ATOM | 3657 | CD1 | LEU | A | 467 | 45.493 | 67.369 | 4.816 | 1.00 | 11.77 | A |
| ATOM | 3658 | CD2 | LEU | A | 467 | 46.359 | 69.555 | 3.975 | 1.00 | 11.14 | A |
| ATOM | 3659 | C | LEU | A | 467 | 44.462 | 66.753 | 1.251 | 1.00 | 7.29 | A |
| ATOM | 3660 | O | LEU | A | 467 | 43.381 | 66.624 | 1.826 | 1.00 | 7.06 | A |
| ATOM | 3661 | N | PHE | A | 468 | 44.570 | 67.270 | 0.029 | 1.00 | 6.97 | A |
| ATOM | 3662 | CA | PHE | A | 468 | 43.392 | 67.756 | −0.680 | 1.00 | 6.88 | A |
| ATOM | 3663 | CB | PHE | A | 468 | 43.800 | 68.553 | −1.925 | 1.00 | 7.64 | A |
| ATOM | 3664 | CG | PHE | A | 468 | 42.688 | 69.391 | −2.491 | 1.00 | 7.18 | A |
| ATOM | 3665 | CD1 | PHE | A | 468 | 42.039 | 70.331 | −1.695 | 1.00 | 7.53 | A |
| ATOM | 3666 | CD2 | PHE | A | 468 | 42.264 | 69.221 | −3.804 | 1.00 | 7.39 | A |
| ATOM | 3667 | CE1 | PHE | A | 468 | 40.979 | 71.089 | −2.198 | 1.00 | 7.30 | A |
| ATOM | 3668 | CE2 | PHE | A | 468 | 41.204 | 69.972 | −4.314 | 1.00 | 7.16 | A |
| ATOM | 3669 | CZ | PHE | A | 468 | 40.561 | 70.907 | −3.510 | 1.00 | 7.44 | A |
| ATOM | 3670 | C | PHE | A | 468 | 42.402 | 66.662 | −1.069 | 1.00 | 6.45 | A |
| ATOM | 3671 | O | PHE | A | 468 | 41.259 | 66.964 | −1.413 | 1.00 | 7.00 | A |
| ATOM | 3672 | N | GLN | A | 469 | 42.829 | 65.401 | −1.018 | 1.00 | 6.03 | A |
| ATOM | 3673 | CA | GLN | A | 469 | 41.931 | 64.302 | −1.352 | 1.00 | 6.57 | A |
| ATOM | 3674 | CB | GLN | A | 469 | 42.707 | 62.998 | −1.542 | 1.00 | 6.70 | A |
| ATOM | 3675 | CG | GLN | A | 469 | 43.795 | 63.094 | −2.600 | 1.00 | 7.58 | A |
| ATOM | 3676 | CD | GLN | A | 469 | 43.304 | 63.745 | −3.878 | 1.00 | 7.69 | A |
| ATOM | 3677 | OE1 | GLN | A | 469 | 43.860 | 64.748 | −4.329 | 1.00 | 9.02 | A |
| ATOM | 3678 | NE2 | GLN | A | 469 | 42.258 | 63.184 | −4.465 | 1.00 | 5.38 | A |
| ATOM | 3679 | C | GLN | A | 469 | 40.898 | 64.121 | −0.250 | 1.00 | 6.50 | A |
| ATOM | 3680 | O | GLN | A | 469 | 39.927 | 63.383 | −0.416 | 1.00 | 7.49 | A |
| ATOM | 3681 | N | HIS | A | 470 | 41.119 | 64.792 | 0.878 | 1.00 | 6.70 | A |
| ATOM | 3682 | CA | HIS | A | 470 | 40.195 | 64.727 | 2.004 | 1.00 | 7.12 | A |
| ATOM | 3683 | CB | HIS | A | 470 | 40.563 | 65.781 | 3.047 | 1.00 | 7.31 | A |
| ATOM | 3684 | CG | HIS | A | 470 | 39.594 | 65.852 | 4.183 | 1.00 | 7.78 | A |
| ATOM | 3685 | CD2 | HIS | A | 470 | 38.969 | 64.869 | 4.872 | 1.00 | 7.00 | A |
| ATOM | 3686 | ND1 | HIS | A | 470 | 39.144 | 67.041 | 4.715 | 1.00 | 9.94 | A |
| ATOM | 3687 | CE1 | HIS | A | 470 | 38.281 | 66.786 | 5.683 | 1.00 | 6.65 | A |
| ATOM | 3688 | NE2 | HIS | A | 470 | 38.157 | 65.476 | 5.797 | 1.00 | 10.50 | A |
| ATOM | 3689 | C | HIS | A | 470 | 38.758 | 64.969 | 1.537 | 1.00 | 6.99 | A |
| ATOM | 3690 | O | HIS | A | 470 | 38.527 | 65.722 | 0.587 | 1.00 | 6.65 | A |
| ATOM | 3691 | N | HIS | A | 471 | 37.793 | 64.351 | 2.215 | 1.00 | 7.65 | A |
| ATOM | 3692 | CA | HIS | A | 471 | 36.398 | 64.503 | 1.824 | 1.00 | 7.92 | A |
| ATOM | 3693 | CB | HIS | A | 471 | 35.532 | 63.380 | 2.417 | 1.00 | 7.70 | A |
| ATOM | 3694 | CG | HIS | A | 471 | 35.636 | 63.249 | 3.901 | 1.00 | 7.25 | A |
| ATOM | 3695 | CD2 | HIS | A | 471 | 34.904 | 63.802 | 4.894 | 1.00 | 8.79 | A |
| ATOM | 3696 | ND1 | HIS | A | 471 | 36.612 | 62.493 | 4.515 | 1.00 | 8.77 | A |
| ATOM | 3697 | CE1 | HIS | A | 471 | 36.476 | 62.589 | 5.825 | 1.00 | 8.02 | A |
| ATOM | 3698 | NE2 | HIS | A | 471 | 35.448 | 63.377 | 6.080 | 1.00 | 8.27 | A |
| ATOM | 3699 | C | HIS | A | 471 | 35.775 | 65.873 | 2.102 | 1.00 | 8.28 | A |
| ATOM | 3700 | O | HIS | A | 471 | 34.566 | 66.039 | 1.965 | 1.00 | 8.06 | A |
| ATOM | 3701 | N | ASP | A | 472 | 36.598 | 66.839 | 2.510 | 1.00 | 8.09 | A |
| ATOM | 3702 | CA | ASP | A | 472 | 36.138 | 68.219 | 2.681 | 1.00 | 8.05 | A |
| ATOM | 3703 | CB | ASP | A | 472 | 36.134 | 68.660 | 4.144 | 1.00 | 9.12 | A |
| ATOM | 3704 | CG | ASP | A | 472 | 35.001 | 68.046 | 4.927 | 1.00 | 8.91 | A |
| ATOM | 3705 | OD1 | ASP | A | 472 | 33.837 | 68.191 | 4.490 | 1.00 | 10.33 | A |
| ATOM | 3706 | OD2 | ASP | A | 472 | 35.274 | 67.426 | 5.974 | 1.00 | 9.95 | A |
| ATOM | 3707 | C | ASP | A | 472 | 37.083 | 69.111 | 1.880 | 1.00 | 8.31 | A |
| ATOM | 3708 | O | ASP | A | 472 | 36.989 | 70.341 | 1.921 | 1.00 | 8.71 | A |
| ATOM | 3709 | N | GLY | A | 473 | 37.991 | 68.476 | 1.143 | 1.00 | 7.76 | A |
| ATOM | 3710 | CA | GLY | A | 473 | 38.941 | 69.214 | 0.329 | 1.00 | 6.95 | A |
| ATOM | 3711 | C | GLY | A | 473 | 38.449 | 69.319 | −1.098 | 1.00 | 6.25 | A |
| ATOM | 3712 | O | GLY | A | 473 | 37.781 | 70.286 | −1.461 | 1.00 | 7.15 | A |
| ATOM | 3713 | N | ILE | A | 474 | 38.753 | 68.306 | −1.904 | 1.00 | 7.12 | A |
| ATOM | 3714 | CA | ILE | A | 474 | 38.345 | 68.297 | −3.302 | 1.00 | 7.43 | A |
| ATOM | 3715 | CB | ILE | A | 474 | 38.883 | 67.023 | −4.011 | 1.00 | 6.98 | A |
| ATOM | 3716 | CG2 | ILE | A | 474 | 38.271 | 65.774 | −3.383 | 1.00 | 7.56 | A |
| ATOM | 3717 | CG1 | ILE | A | 474 | 38.604 | 67.097 | −5.514 | 1.00 | 7.17 | A |
| ATOM | 3718 | CD1 | ILE | A | 474 | 39.279 | 65.983 | −6.308 | 1.00 | 9.11 | A |
| ATOM | 3719 | C | ILE | A | 474 | 36.824 | 68.408 | −3.468 | 1.00 | 8.30 | A |
| ATOM | 3720 | O | ILE | A | 474 | 36.334 | 68.865 | −4.500 | 1.00 | 8.97 | A |
| ATOM | 3721 | N | THR | A | 475 | 36.087 | 68.009 | −2.436 | 1.00 | 7.83 | A |
| ATOM | 3722 | CA | THR | A | 475 | 34.625 | 68.054 | −2.440 | 1.00 | 8.11 | A |
| ATOM | 3723 | CB | THR | A | 475 | 34.066 | 67.378 | −1.189 | 1.00 | 8.53 | A |
| ATOM | 3724 | OG1 | THR | A | 475 | 34.589 | 68.053 | −0.038 | 1.00 | 8.85 | A |
| ATOM | 3725 | CG2 | THR | A | 475 | 34.458 | 65.905 | −1.139 | 1.00 | 9.50 | A |
| ATOM | 3726 | C | THR | A | 475 | 34.067 | 69.482 | −2.456 | 1.00 | 8.06 | A |
| ATOM | 3727 | O | THR | A | 475 | 32.906 | 69.702 | −2.817 | 1.00 | 8.47 | A |
| ATOM | 3728 | N | GLY | A | 476 | 34.888 | 70.448 | −2.051 | 1.00 | 7.78 | A |
| ATOM | 3729 | CA | GLY | A | 476 | 34.435 | 71.828 | −2.020 | 1.00 | 8.58 | A |
| ATOM | 3730 | C | GLY | A | 476 | 33.424 | 72.078 | −0.914 | 1.00 | 8.79 | A |
| ATOM | 3731 | O | GLY | A | 476 | 32.534 | 72.917 | −1.062 | 1.00 | 8.66 | A |
| ATOM | 3732 | N | THR | A | 477 | 33.558 | 71.354 | 0.195 | 1.00 | 8.54 | A |
| ATOM | 3733 | CA | THR | A | 477 | 32.634 | 71.502 | 1.315 | 1.00 | 9.22 | A |
| ATOM | 3734 | CB | THR | A | 477 | 32.012 | 70.134 | 1.700 | 1.00 | 8.79 | A |
| ATOM | 3735 | OG1 | THR | A | 477 | 33.053 | 69.184 | 1.974 | 1.00 | 8.67 | A |
| ATOM | 3736 | CG2 | THR | A | 477 | 31.150 | 69.610 | 0.558 | 1.00 | 9.58 | A |
| ATOM | 3737 | C | THR | A | 477 | 33.239 | 72.144 | 2.567 | 1.00 | 9.40 | A |
| ATOM | 3738 | O | THR | A | 477 | 32.671 | 72.034 | 3.655 | 1.00 | 10.45 | A |
| ATOM | 3739 | N | ALA | A | 478 | 34.374 | 72.826 | 2.417 | 1.00 | 9.36 | A |
| ATOM | 3740 | CA | ALA | A | 478 | 35.022 | 73.479 | 3.558 | 1.00 | 9.20 | A |
| ATOM | 3741 | CB | ALA | A | 478 | 36.535 | 73.254 | 3.501 | 1.00 | 10.02 | A |
| ATOM | 3742 | C | ALA | A | 478 | 34.722 | 74.979 | 3.619 | 1.00 | 9.62 | A |
| ATOM | 3743 | O | ALA | A | 478 | 34.225 | 75.572 | 2.657 | 1.00 | 8.69 | A |
| ATOM | 3744 | N | LYS | A | 479 | 35.016 | 75.598 | 4.759 | 1.00 | 9.96 | A |
| ATOM | 3745 | CA | LYS | A | 479 | 34.775 | 77.030 | 4.888 | 1.00 | 10.67 | A |
| ATOM | 3746 | CB | LYS | A | 479 | 34.961 | 77.492 | 6.337 | 1.00 | 12.18 | A |
| ATOM | 3747 | CG | LYS | A | 479 | 33.942 | 76.890 | 7.300 | 1.00 | 15.03 | A |
| ATOM | 3748 | CD | LYS | A | 479 | 34.018 | 77.529 | 8.677 | 1.00 | 18.09 | A |
| ATOM | 3749 | CE | LYS | A | 479 | 33.187 | 78.798 | 8.755 | 1.00 | 20.24 | A |
| ATOM | 3750 | NZ | LYS | A | 479 | 31.717 | 78.517 | 8.701 | 1.00 | 19.56 | A |
| ATOM | 3751 | C | LYS | A | 479 | 35.746 | 77.761 | 3.972 | 1.00 | 10.66 | A |
| ATOM | 3752 | O | LYS | A | 479 | 36.793 | 77.225 | 3.607 | 1.00 | 10.46 | A |
| ATOM | 3753 | N | THR | A | 480 | 35.393 | 78.984 | 3.603 | 1.00 | 11.10 | A |
| ATOM | 3754 | CA | THR | A | 480 | 36.214 | 79.784 | 2.707 | 1.00 | 11.50 | A |
| ATOM | 3755 | CB | THR | A | 480 | 35.646 | 81.208 | 2.584 | 1.00 | 13.37 | A |
| ATOM | 3756 | OG1 | THR | A | 480 | 34.331 | 81.139 | 2.021 | 1.00 | 14.84 | A |
| ATOM | 3757 | CG2 | THR | A | 480 | 36.529 | 82.068 | 1.683 | 1.00 | 13.82 | A |
| ATOM | 3758 | C | THR | A | 480 | 37.696 | 79.870 | 3.059 | 1.00 | 11.02 | A |
| ATOM | 3759 | O | THR | A | 480 | 38.548 | 79.651 | 2.192 | 1.00 | 10.46 | A |
| ATOM | 3760 | N | HIS | A | 481 | 38.025 | 80.179 | 4.310 | 1.00 | 10.35 | A |
| ATOM | 3761 | CA | HIS | A | 481 | 39.437 | 80.296 | 4.655 | 1.00 | 9.50 | A |
| ATOM | 3762 | CB | HIS | A | 481 | 39.621 | 80.983 | 6.023 | 1.00 | 10.43 | A |
| ATOM | 3763 | CG | HIS | A | 481 | 39.453 | 80.077 | 7.204 | 1.00 | 10.15 | A |
| ATOM | 3764 | CD2 | HIS | A | 481 | 40.366 | 79.556 | 8.057 | 1.00 | 10.66 | A |
| ATOM | 3765 | ND1 | HIS | A | 481 | 38.223 | 79.632 | 7.641 | 1.00 | 11.08 | A |
| ATOM | 3766 | CE1 | HIS | A | 481 | 38.387 | 78.878 | 8.712 | 1.00 | 11.48 | A |
| ATOM | 3767 | NE2 | HIS | A | 481 | 39.678 | 78.816 | 8.986 | 1.00 | 10.90 | A |
| ATOM | 3768 | C | HIS | A | 481 | 40.169 | 78.955 | 4.610 | 1.00 | 9.97 | A |
| ATOM | 3769 | O | HIS | A | 481 | 41.390 | 78.914 | 4.454 | 1.00 | 10.08 | A |
| ATOM | 3770 | N | VAL | A | 482 | 39.423 | 77.859 | 4.726 | 1.00 | 9.90 | A |
| ATOM | 3771 | CA | VAL | A | 482 | 40.030 | 76.534 | 4.671 | 1.00 | 9.55 | A |
| ATOM | 3772 | CB | VAL | A | 482 | 39.103 | 75.471 | 5.296 | 1.00 | 8.77 | A |
| ATOM | 3773 | CG1 | VAL | A | 482 | 39.771 | 74.101 | 5.263 | 1.00 | 8.62 | A |
| ATOM | 3774 | CG2 | VAL | A | 482 | 38.794 | 75.852 | 6.744 | 1.00 | 9.85 | A |
| ATOM | 3775 | C | VAL | A | 482 | 40.327 | 76.206 | 3.206 | 1.00 | 9.39 | A |
| ATOM | 3776 | O | VAL | A | 482 | 41.374 | 75.646 | 2.886 | 1.00 | 9.70 | A |
| ATOM | 3777 | N | VAL | A | 483 | 39.405 | 76.568 | 2.319 | 1.00 | 9.35 | A |
| ATOM | 3778 | CA | VAL | A | 483 | 39.612 | 76.356 | 0.893 | 1.00 | 9.49 | A |
| ATOM | 3779 | CB | VAL | A | 483 | 38.412 | 76.872 | 0.075 | 1.00 | 9.54 | A |
| ATOM | 3780 | CG1 | VAL | A | 483 | 38.728 | 76.821 | −1.412 | 1.00 | 10.64 | A |
| ATOM | 3781 | CG2 | VAL | A | 483 | 37.182 | 76.023 | 0.377 | 1.00 | 10.55 | A |
| ATOM | 3782 | C | VAL | A | 483 | 40.873 | 77.126 | 0.487 | 1.00 | 9.63 | A |
| ATOM | 3783 | O | VAL | A | 483 | 41.693 | 76.636 | −0.290 | 1.00 | 9.14 | A |
| ATOM | 3784 | N | VAL | A | 484 | 41.034 | 78.328 | 1.037 | 1.00 | 10.23 | A |
| ATOM | 3785 | CA | VAL | A | 484 | 42.203 | 79.142 | 0.734 | 1.00 | 10.83 | A |
| ATOM | 3786 | CB | VAL | A | 484 | 42.112 | 80.524 | 1.410 | 1.00 | 10.78 | A |
| ATOM | 3787 | CG1 | VAL | A | 484 | 43.445 | 81.261 | 1.292 | 1.00 | 11.71 | A |
| ATOM | 3788 | CG2 | VAL | A | 484 | 41.009 | 81.333 | 0.754 | 1.00 | 11.99 | A |
| ATOM | 3789 | C | VAL | A | 484 | 43.477 | 78.442 | 1.187 | 1.00 | 10.65 | A |
| ATOM | 3790 | O | VAL | A | 484 | 44.488 | 78.473 | 0.484 | 1.00 | 11.13 | A |
| ATOM | 3791 | N | ASP | A | 485 | 43.431 | 77.809 | 2.357 | 1.00 | 9.92 | A |
| ATOM | 3792 | CA | ASP | A | 485 | 44.596 | 77.094 | 2.862 | 1.00 | 9.04 | A |
| ATOM | 3793 | CB | ASP | A | 485 | 44.331 | 76.550 | 4.271 | 1.00 | 10.28 | A |
| ATOM | 3794 | CG | ASP | A | 485 | 45.571 | 75.944 | 4.904 | 1.00 | 10.84 | A |
| ATOM | 3795 | OD1 | ASP | A | 485 | 45.531 | 74.760 | 5.291 | 1.00 | 11.04 | A |
| ATOM | 3796 | OD2 | ASP | A | 485 | 46.592 | 76.657 | 5.013 | 1.00 | 13.22 | A |
| ATOM | 3797 | C | ASP | A | 485 | 44.960 | 75.945 | 1.922 | 1.00 | 8.24 | A |
| ATOM | 3798 | O | ASP | A | 485 | 46.131 | 75.739 | 1.613 | 1.00 | 8.35 | A |
| ATOM | 3799 | N | TYR | A | 486 | 43.959 | 75.192 | 1.474 | 1.00 | 8.00 | A |
| ATOM | 3800 | CA | TYR | A | 486 | 44.211 | 74.088 | 0.550 | 1.00 | 8.25 | A |
| ATOM | 3801 | CB | TYR | A | 486 | 42.912 | 73.357 | 0.205 | 1.00 | 8.89 | A |
| ATOM | 3802 | CG | TYR | A | 486 | 42.326 | 72.506 | 1.311 | 1.00 | 8.67 | A |
| ATOM | 3803 | CD1 | TYR | A | 486 | 40.953 | 72.504 | 1.542 | 1.00 | 8.68 | A |
| ATOM | 3804 | CE1 | TYR | A | 486 | 40.386 | 71.694 | 2.520 | 1.00 | 8.58 | A |
| ATOM | 3805 | CD2 | TYR | A | 486 | 43.131 | 71.669 | 2.095 | 1.00 | 9.01 | A |
| ATOM | 3806 | CE2 | TYR | A | 486 | 42.567 | 70.848 | 3.085 | 1.00 | 9.81 | A |
| ATOM | 3807 | CZ | TYR | A | 486 | 41.192 | 70.872 | 3.284 | 1.00 | 9.19 | A |
| ATOM | 3808 | OH | TYR | A | 486 | 40.607 | 70.075 | 4.245 | 1.00 | 10.42 | A |
| ATOM | 3809 | C | TYR | A | 486 | 44.831 | 74.618 | −0.740 | 1.00 | 9.13 | A |
| ATOM | 3810 | O | TYR | A | 486 | 45.748 | 74.009 | −1.283 | 1.00 | 8.83 | A |
| ATOM | 3811 | N | GLU | A | 487 | 44.324 | 75.748 | −1.229 | 1.00 | 9.52 | A |
| ATOM | 3812 | CA | GLU | A | 487 | 44.841 | 76.345 | −2.456 | 1.00 | 10.36 | A |
| ATOM | 3813 | CB | GLU | A | 487 | 44.007 | 77.563 | −2.865 | 1.00 | 11.32 | A |
| ATOM | 3814 | CG | GLU | A | 487 | 44.321 | 78.044 | −4.282 | 1.00 | 13.08 | A |
| ATOM | 3815 | CD | GLU | A | 487 | 43.569 | 79.300 | −4.677 | 1.00 | 15.53 | A |
| ATOM | 3816 | OE1 | GLU | A | 487 | 42.384 | 79.441 | −4.310 | 1.00 | 16.78 | A |
| ATOM | 3817 | OE2 | GLU | A | 487 | 44.163 | 80.147 | −5.377 | 1.00 | 17.62 | A |
| ATOM | 3818 | C | GLU | A | 487 | 46.296 | 76.771 | −2.293 | 1.00 | 11.00 | A |
| ATOM | 3819 | O | GLU | A | 487 | 47.125 | 76.522 | −3.168 | 1.00 | 10.62 | A |
| ATOM | 3820 | N | GLN | A | 488 | 46.601 | 77.423 | −1.175 | 1.00 | 11.10 | A |
| ATOM | 3821 | CA | GLN | A | 488 | 47.962 | 77.875 | −0.910 | 1.00 | 12.20 | A |
| ATOM | 3822 | CB | GLN | A | 488 | 48.031 | 78.621 | 0.425 | 1.00 | 15.17 | A |
| ATOM | 3823 | CG | GLN | A | 488 | 47.343 | 79.970 | 0.426 | 1.00 | 19.59 | A |
| ATOM | 3824 | CD | GLN | A | 488 | 47.395 | 80.642 | 1.782 | 1.00 | 21.97 | A |
| ATOM | 3825 | OE1 | GLN | A | 488 | 47.075 | 81.821 | 1.912 | 1.00 | 25.37 | A |
| ATOM | 3826 | NE2 | GLN | A | 488 | 47.795 | 79.890 | 2.804 | 1.00 | 24.06 | A |
| ATOM | 3827 | C | GLN | A | 488 | 48.904 | 76.684 | −0.865 | 1.00 | 11.05 | A |
| ATOM | 3828 | O | GLN | A | 488 | 50.000 | 76.727 | −1.421 | 1.00 | 10.97 | A |
| ATOM | 3829 | N | ARG | A | 489 | 48.473 | 75.621 | −0.190 | 1.00 | 9.60 | A |
| ATOM | 3830 | CA | ARG | A | 489 | 49.278 | 74.416 | −0.083 | 1.00 | 9.37 | A |
| ATOM | 3831 | CB | ARG | A | 489 | 48.571 | 73.385 | 0.804 | 1.00 | 9.55 | A |
| ATOM | 3832 | CG | ARG | A | 489 | 48.601 | 73.714 | 2.294 | 1.00 | 10.40 | A |
| ATOM | 3833 | CD | ARG | A | 489 | 47.660 | 72.790 | 3.063 | 1.00 | 10.86 | A |
| ATOM | 3834 | NE | ARG | A | 489 | 47.738 | 72.969 | 4.513 | 1.00 | 11.17 | A |
| ATOM | 3835 | CZ | ARG | A | 489 | 48.620 | 72.361 | 5.302 | 1.00 | 12.00 | A |
| ATOM | 3836 | NH1 | ARG | A | 489 | 49.513 | 71.522 | 4.792 | 1.00 | 13.64 | A |
| ATOM | 3837 | NH2 | ARG | A | 489 | 48.606 | 72.593 | 6.610 | 1.00 | 12.68 | A |
| ATOM | 3838 | C | ARG | A | 489 | 49.528 | 73.835 | −1.472 | 1.00 | 9.02 | A |
| ATOM | 3839 | O | ARG | A | 489 | 50.656 | 73.468 | −1.801 | 1.00 | 8.75 | A |
| ATOM | 3840 | N | MSE | A | 490 | 48.487 | 73.766 | −2.297 | 1.00 | 9.23 | A |
| ATOM | 3841 | CA | MSE | A | 490 | 48.666 | 73.224 | −3.639 | 1.00 | 9.79 | A |
| ATOM | 3842 | CB | MSE | A | 490 | 47.314 | 72.964 | −4.314 | 1.00 | 10.83 | A |
| ATOM | 3843 | CG | MSE | A | 490 | 46.571 | 71.768 | −3.719 | 1.00 | 11.89 | A |
| ATOM | 3844 | SE | MSE | A | 490 | 45.198 | 71.045 | −4.876 | 1.00 | 18.09 | A |
| ATOM | 3845 | CE | MSE | A | 490 | 43.813 | 72.317 | −4.470 | 1.00 | 13.98 | A |
| ATOM | 3846 | C | MSE | A | 490 | 49.539 | 74.117 | −4.519 | 1.00 | 10.09 | A |
| ATOM | 3847 | O | MSE | A | 490 | 50.254 | 73.620 | −5.387 | 1.00 | 9.19 | A |
| ATOM | 3848 | N | GLN | A | 491 | 49.490 | 75.427 | −4.300 | 1.00 | 10.39 | A |
| ATOM | 3849 | CA | GLN | A | 491 | 50.314 | 76.332 | −5.093 | 1.00 | 12.11 | A |
| ATOM | 3850 | CB | GLN | A | 491 | 49.972 | 77.790 | −4.786 | 1.00 | 14.32 | A |
| ATOM | 3851 | CG | GLN | A | 491 | 50.755 | 78.791 | −5.620 | 1.00 | 19.08 | A |
| ATOM | 3852 | CD | GLN | A | 491 | 50.647 | 78.520 | −7.109 | 1.00 | 21.56 | A |
| ATOM | 3853 | OE1 | GLN | A | 491 | 51.427 | 77.746 | −7.676 | 1.00 | 23.81 | A |
| ATOM | 3854 | NE2 | GLN | A | 491 | 49.669 | 79.145 | −7.750 | 1.00 | 23.81 | A |
| ATOM | 3855 | C | GLN | A | 491 | 51.779 | 76.064 | −4.769 | 1.00 | 11.74 | A |
| ATOM | 3856 | O | GLN | A | 491 | 52.630 | 76.040 | −5.658 | 1.00 | 10.95 | A |
| ATOM | 3857 | N | GLU | A | 492 | 52.068 | 75.851 | −3.489 | 1.00 | 11.42 | A |
| ATOM | 3858 | CA | GLU | A | 492 | 53.432 | 75.570 | −3.066 | 1.00 | 12.73 | A |
| ATOM | 3859 | CB | GLU | A | 492 | 53.511 | 75.506 | −1.536 | 1.00 | 15.62 | A |
| ATOM | 3860 | CG | GLU | A | 492 | 52.957 | 76.743 | −0.844 | 1.00 | 21.31 | A |
| ATOM | 3861 | CD | GLU | A | 492 | 53.040 | 76.666 | 0.668 | 1.00 | 24.20 | A |
| ATOM | 3862 | OE1 | GLU | A | 492 | 52.577 | 75.660 | 1.249 | 1.00 | 26.61 | A |
| ATOM | 3863 | OE2 | GLU | A | 492 | 53.564 | 77.622 | 1.280 | 1.00 | 27.33 | A |
| ATOM | 3864 | C | GLU | A | 492 | 53.863 | 74.236 | −3.669 | 1.00 | 10.97 | A |
| ATOM | 3865 | O | GLU | A | 492 | 55.014 | 74.065 | −4.073 | 1.00 | 11.10 | A |
| ATOM | 3866 | N | ALA | A | 493 | 52.929 | 73.292 | −3.734 | 1.00 | 9.75 | A |
| ATOM | 3867 | CA | ALA | A | 493 | 53.215 | 71.979 | −4.301 | 1.00 | 8.02 | A |
| ATOM | 3868 | CB | ALA | A | 493 | 52.018 | 71.048 | −4.097 | 1.00 | 8.73 | A |
| ATOM | 3869 | C | ALA | A | 493 | 53.544 | 72.100 | −5.792 | 1.00 | 8.05 | A |
| ATOM | 3870 | O | ALA | A | 493 | 54.453 | 71.436 | −6.290 | 1.00 | 7.12 | A |
| ATOM | 3871 | N | LEU | A | 494 | 52.802 | 72.948 | −6.501 | 1.00 | 8.32 | A |
| ATOM | 3872 | CA | LEU | A | 494 | 53.046 | 73.145 | −7.925 | 1.00 | 9.14 | A |
| ATOM | 3873 | CB | LEU | A | 494 | 51.979 | 74.069 | −8.530 | 1.00 | 9.65 | A |
| ATOM | 3874 | CG | LEU | A | 494 | 50.611 | 73.420 | −8.773 | 1.00 | 10.02 | A |
| ATOM | 3875 | CD1 | LEU | A | 494 | 49.579 | 74.489 | −9.132 | 1.00 | 10.77 | A |
| ATOM | 3876 | CD2 | LEU | A | 494 | 50.729 | 72.390 | −9.889 | 1.00 | 10.73 | A |
| ATOM | 3877 | C | LEU | A | 494 | 54.437 | 73.726 | −8.145 | 1.00 | 9.07 | A |
| ATOM | 3878 | O | LEU | A | 494 | 55.158 | 73.297 | −9.043 | 1.00 | 9.80 | A |
| ATOM | 3879 | N | LYS | A | 495 | 54.816 | 74.694 | −7.315 | 1.00 | 9.88 | A |
| ATOM | 3880 | CA | LYS | A | 495 | 56.137 | 75.307 | −7.427 | 1.00 | 10.44 | A |
| ATOM | 3881 | CB | LYS | A | 495 | 56.260 | 76.494 | −6.467 | 1.00 | 12.88 | A |
| ATOM | 3882 | CG | LYS | A | 495 | 55.346 | 77.657 | −6.831 | 1.00 | 16.66 | A |
| ATOM | 3883 | CD | LYS | A | 495 | 55.583 | 78.879 | −5.955 | 1.00 | 19.89 | A |
| ATOM | 3884 | CE | LYS | A | 495 | 55.238 | 78.611 | −4.501 | 1.00 | 21.23 | A |
| ATOM | 3885 | NZ | LYS | A | 495 | 55.445 | 79.824 | −3.656 | 1.00 | 23.31 | A |
| ATOM | 3886 | C | LYS | A | 495 | 57.221 | 74.273 | −7.138 | 1.00 | 10.02 | A |
| ATOM | 3887 | O | LYS | A | 495 | 58.268 | 74.260 | −7.788 | 1.00 | 10.29 | A |
| ATOM | 3888 | N | ALA | A | 496 | 56.965 | 73.397 | −6.170 | 1.00 | 8.63 | A |
| ATOM | 3889 | CA | ALA | A | 496 | 57.923 | 72.351 | −5.832 | 1.00 | 8.27 | A |
| ATOM | 3890 | CB | ALA | A | 496 | 57.433 | 71.564 | −4.613 | 1.00 | 9.05 | A |
| ATOM | 3891 | C | ALA | A | 496 | 58.094 | 71.419 | −7.037 | 1.00 | 8.71 | A |
| ATOM | 3892 | O | ALA | A | 496 | 59.211 | 71.028 | −7.387 | 1.00 | 8.19 | A |
| ATOM | 3893 | N | CYS | A | 497 | 56.982 | 71.068 | −7.676 | 1.00 | 8.28 | A |
| ATOM | 3894 | CA | CYS | A | 497 | 57.032 | 70.192 | −8.838 | 1.00 | 8.42 | A |
| ATOM | 3895 | CB | CYS | A | 497 | 55.618 | 69.845 | −9.313 | 1.00 | 7.83 | A |
| ATOM | 3896 | SG | CYS | A | 497 | 54.759 | 68.649 | −8.261 | 1.00 | 8.61 | A |
| ATOM | 3897 | C | CYS | A | 497 | 57.813 | 70.848 | −9.970 | 1.00 | 8.81 | A |
| ATOM | 3898 | O | CYS | A | 497 | 58.639 | 70.207 | −10.615 | 1.00 | 8.25 | A |
| ATOM | 3899 | N | GLN | A | 498 | 57.550 | 72.126 | −10.212 | 1.00 | 8.64 | A |
| ATOM | 3900 | CA | GLN | A | 498 | 58.248 | 72.843 | −11.272 | 1.00 | 10.15 | A |
| ATOM | 3901 | GB | GLN | A | 498 | 57.765 | 74.293 | −11.355 | 1.00 | 11.26 | A |
| ATOM | 3902 | CG | GLN | A | 498 | 58.576 | 75.132 | −12.335 | 1.00 | 14.98 | A |
| ATOM | 3903 | CD | GLN | A | 498 | 58.111 | 76.573 | −12.423 | 1.00 | 17.06 | A |
| ATOM | 3904 | OE1 | GLN | A | 498 | 58.033 | 77.280 | −11.412 | 1.00 | 19.34 | A |
| ATOM | 3905 | NE2 | GLN | A | 498 | 57.814 | 77.023 | −13.636 | 1.00 | 16.12 | A |
| ATOM | 3906 | C | GLN | A | 498 | 59.755 | 72.824 | −11.040 | 1.00 | 10.31 | A |
| ATOM | 3907 | O | GLN | A | 498 | 60.532 | 72.590 | −11.966 | 1.00 | 9.65 | A |
| ATOM | 3908 | N | MSE | A | 499 | 60.165 | 73.080 | −9.801 | 1.00 | 9.78 | A |
| ATOM | 3909 | CA | MSE | A | 499 | 61.580 | 73.090 | −9.457 | 1.00 | 11.48 | A |
| ATOM | 3910 | CB | MSE | A | 499 | 61.751 | 73.411 | −7.968 | 1.00 | 15.37 | A |
| ATOM | 3911 | CG | MSE | A | 499 | 63.163 | 73.223 | −7.420 | 1.00 | 19.93 | A |
| ATOM | 3912 | SE | MSE | A | 499 | 64.512 | 74.312 | −8.276 | 1.00 | 30.03 | A |
| ATOM | 3913 | CE | MSE | A | 499 | 63.919 | 76.038 | −7.664 | 1.00 | 24.23 | A |
| ATOM | 3914 | C | MSE | A | 499 | 62.226 | 71.748 | −9.783 | 1.00 | 10.06 | A |
| ATOM | 3915 | O | MSE | A | 499 | 63.261 | 71.689 | −10.445 | 1.00 | 9.59 | A |
| ATOM | 3916 | N | VAL | A | 500 | 61.602 | 70.665 | −9.328 | 1.00 | 9.10 | A |
| ATOM | 3917 | CA | VAL | A | 500 | 62.134 | 69.336 | −9.572 | 1.00 | 8.89 | A |
| ATOM | 3918 | CB | VAL | A | 500 | 61.310 | 68.274 | −8.814 | 1.00 | 8.59 | A |
| ATOM | 3919 | CG1 | VAL | A | 500 | 61.761 | 66.871 | −9.206 | 1.00 | 8.21 | A |
| ATOM | 3920 | CG2 | VAL | A | 500 | 61.473 | 68.486 | −7.316 | 1.00 | 10.28 | A |
| ATOM | 3921 | C | VAL | A | 500 | 62.156 | 69.009 | −11.062 | 1.00 | 8.51 | A |
| ATOM | 3922 | O | VAL | A | 500 | 63.149 | 68.495 | −11.573 | 1.00 | 9.06 | A |
| ATOM | 3923 | N | MSE | A | 501 | 61.063 | 69.307 | −11.752 | 1.00 | 8.86 | A |
| ATOM | 3924 | CA | MSE | A | 501 | 60.975 | 69.037 | −13.181 | 1.00 | 8.89 | A |
| ATOM | 3925 | CB | MSE | A | 501 | 59.589 | 69.427 | −13.698 | 1.00 | 11.27 | A |
| ATOM | 3926 | CG | MSE | A | 501 | 58.472 | 68.543 | −13.164 | 1.00 | 12.53 | A |
| ATOM | 3927 | SE | MSE | A | 501 | 56.723 | 69.312 | −13.426 | 1.00 | 20.16 | A |
| ATOM | 3928 | CE | MSE | A | 501 | 56.544 | 69.004 | −15.314 | 1.00 | 15.92 | A |
| ATOM | 3929 | C | MSE | A | 501 | 62.051 | 69.751 | −13.993 | 1.00 | 8.69 | A |
| ATOM | 3930 | O | MSE | A | 501 | 62.750 | 69.124 | −14.793 | 1.00 | 8.53 | A |
| ATOM | 3931 | N | GLN | A | 502 | 62.193 | 71.057 | −13.793 | 1.00 | 9.61 | A |
| ATOM | 3932 | CA | GLN | A | 502 | 63.183 | 71.813 | −14.551 | 1.00 | 9.86 | A |
| ATOM | 3933 | CB | GLN | A | 502 | 62.972 | 73.318 | −14.351 | 1.00 | 10.38 | A |
| ATOM | 3934 | CG | GLN | A | 502 | 63.229 | 73.852 | −12.958 | 1.00 | 10.99 | A |
| ATOM | 3935 | CD | GLN | A | 502 | 64.644 | 74.356 | −12.798 | 1.00 | 11.92 | A |
| ATOM | 3936 | OE1 | GLN | A | 502 | 65.370 | 74.510 | −13.782 | 1.00 | 11.67 | A |
| ATOM | 3937 | NE2 | GLN | A | 502 | 65.042 | 74.633 | −11.563 | 1.00 | 12.62 | A |
| ATOM | 3938 | C | GLN | A | 502 | 64.620 | 71.396 | −14.236 | 1.00 | 10.24 | A |
| ATOM | 3939 | O | GLN | A | 502 | 65.463 | 71.366 | −15.131 | 1.00 | 9.31 | A |
| ATOM | 3940 | N | GLN | A | 503 | 64.909 | 71.062 | −12.980 | 1.00 | 9.34 | A |
| ATOM | 3941 | CA | GLN | A | 503 | 66.260 | 70.608 | −12.642 | 1.00 | 10.05 | A |
| ATOM | 3942 | CB | GLN | A | 503 | 66.408 | 70.369 | −11.135 | 1.00 | 10.04 | A |
| ATOM | 3943 | CG | GLN | A | 503 | 66.626 | 71.616 | −10.285 | 1.00 | 11.30 | A |
| ATOM | 3944 | CD | GLN | A | 503 | 67.985 | 72.265 | −10.503 | 1.00 | 12.37 | A |
| ATOM | 3945 | OE1 | GLN | A | 503 | 68.979 | 71.588 | −10.775 | 1.00 | 13.68 | A |
| ATOM | 3946 | NE2 | GLN | A | 503 | 68.035 | 73.582 | −10.354 | 1.00 | 12.98 | A |
| ATOM | 3947 | C | GLN | A | 503 | 66.520 | 69.291 | −13.374 | 1.00 | 9.96 | A |
| ATOM | 3948 | O | GLN | A | 503 | 67.617 | 69.048 | −13.876 | 1.00 | 10.50 | A |
| ATOM | 3949 | N | SER | A | 504 | 65.502 | 68.435 | −13.425 | 1.00 | 9.59 | A |
| ATOM | 3950 | CA | SER | A | 504 | 65.628 | 67.141 | −14.085 | 1.00 | 9.96 | A |
| ATOM | 3951 | CB | SER | A | 504 | 64.380 | 66.291 | −13.824 | 1.00 | 10.16 | A |
| ATOM | 3952 | OG | SER | A | 504 | 64.243 | 66.012 | −12.440 | 1.00 | 9.72 | A |
| ATOM | 3953 | C | SER | A | 504 | 65.848 | 67.280 | −15.584 | 1.00 | 9.31 | A |
| ATOM | 3954 | O | SER | A | 504 | 66.690 | 66.589 | −16.159 | 1.00 | 8.89 | A |
| ATOM | 3955 | N | VAL | A | 505 | 65.089 | 68.167 | −16.219 | 1.00 | 9.28 | A |
| ATOM | 3956 | CA | VAL | A | 505 | 65.229 | 68.371 | −17.655 | 1.00 | 9.59 | A |
| ATOM | 3957 | CB | VAL | A | 505 | 64.202 | 69.399 | −18.175 | 1.00 | 9.08 | A |
| ATOM | 3958 | CG1 | VAL | A | 505 | 64.522 | 69.782 | −19.618 | 1.00 | 9.37 | A |
| ATOM | 3959 | CG2 | VAL | A | 505 | 62.800 | 68.807 | −18.095 | 1.00 | 9.95 | A |
| ATOM | 3960 | C | VAL | A | 505 | 66.640 | 68.849 | −17.987 | 1.00 | 9.71 | A |
| ATOM | 3961 | O | VAL | A | 505 | 67.260 | 68.376 | −18.940 | 1.00 | 10.10 | A |
| ATOM | 3962 | N | TYR | A | 506 | 67.154 | 69.778 | −17.193 | 1.00 | 9.87 | A |
| ATOM | 3963 | CA | TYR | A | 506 | 68.492 | 70.293 | −17.438 | 1.00 | 11.11 | A |
| ATOM | 3964 | CB | TYR | A | 506 | 68.830 | 71.384 | −16.423 | 1.00 | 12.61 | A |
| ATOM | 3965 | CG | TYR | A | 506 | 70.105 | 72.117 | −16.747 | 1.00 | 14.68 | A |
| ATOM | 3966 | CD1 | TYR | A | 506 | 70.239 | 72.809 | −17.951 | 1.00 | 15.77 | A |
| ATOM | 3967 | CE1 | TYR | A | 506 | 71.413 | 73.469 | −18.275 | 1.00 | 17.48 | A |
| ATOM | 3968 | CD2 | TYR | A | 506 | 71.184 | 72.103 | −15.868 | 1.00 | 15.76 | A |
| ATOM | 3969 | CE2 | TYR | A | 506 | 72.375 | 72.762 | −16.185 | 1.00 | 17.87 | A |
| ATOM | 3970 | CZ | TYR | A | 506 | 72.475 | 73.442 | −17.390 | 1.00 | 17.40 | A |
| ATOM | 3971 | OH | TYR | A | 506 | 73.631 | 74.107 | −17.715 | 1.00 | 19.63 | A |
| ATOM | 3972 | C | TYR | A | 506 | 69.536 | 69.173 | −17.370 | 1.00 | 11.07 | A |
| ATOM | 3973 | O | TYR | A | 506 | 70.433 | 69.091 | −18.208 | 1.00 | 11.91 | A |
| ATOM | 3974 | N | ARG | A | 507 | 69.409 | 68.297 | −16.381 | 1.00 | 10.61 | A |
| ATOM | 3975 | CA | ARG | A | 507 | 70.360 | 67.198 | −16.233 | 1.00 | 10.72 | A |
| ATOM | 3976 | CB | ARG | A | 507 | 70.183 | 66.538 | −14.863 | 1.00 | 11.26 | A |
| ATOM | 3977 | CG | ARG | A | 507 | 71.244 | 65.498 | −14.526 | 1.00 | 12.91 | A |
| ATOM | 3978 | CD | ARG | A | 507 | 71.069 | 64.994 | −13.106 | 1.00 | 13.23 | A |
| ATOM | 3979 | NE | ARG | A | 507 | 72.063 | 63.986 | −12.746 | 1.00 | 15.07 | A |
| ATOM | 3980 | CZ | ARG | A | 507 | 72.164 | 63.442 | −11.536 | 1.00 | 15.08 | A |
| ATOM | 3981 | NH1 | ARG | A | 507 | 71.334 | 63.813 | −10.572 | 1.00 | 15.72 | A |
| ATOM | 3982 | NH2 | ARG | A | 507 | 73.081 | 62.514 | −11.294 | 1.00 | 16.84 | A |
| ATOM | 3983 | C | ARG | A | 507 | 70.219 | 66.148 | −17.339 | 1.00 | 10.75 | A |
| ATOM | 3984 | O | ARG | A | 507 | 71.211 | 65.609 | −17.829 | 1.00 | 10.91 | A |
| ATOM | 3985 | N | LEU | A | 508 | 68.983 | 65.863 | −17.734 | 1.00 | 9.85 | A |
| ATOM | 3986 | CA | LEU | A | 508 | 68.726 | 64.865 | −18.769 | 1.00 | 10.20 | A |
| ATOM | 3987 | CB | LEU | A | 508 | 67.242 | 64.483 | −18.766 | 1.00 | 9.54 | A |
| ATOM | 3988 | CG | LEU | A | 508 | 66.755 | 63.648 | −17.574 | 1.00 | 9.24 | A |
| ATOM | 3989 | CD1 | LEU | A | 508 | 65.238 | 63.747 | −17.461 | 1.00 | 9.92 | A |
| ATOM | 3990 | CD2 | LEU | A | 508 | 67.205 | 62.206 | −17.737 | 1.00 | 10.33 | A |
| ATOM | 3991 | C | LEU | A | 508 | 69.120 | 65.287 | −20.182 | 1.00 | 9.83 | A |
| ATOM | 3992 | O | LEU | A | 508 | 69.387 | 64.434 | −21.035 | 1.00 | 10.94 | A |
| ATOM | 3993 | N | LEU | A | 509 | 69.159 | 66.593 | −20.436 | 1.00 | 10.67 | A |
| ATOM | 3994 | CA | LEU | A | 509 | 69.484 | 67.089 | −21.768 | 1.00 | 10.38 | A |
| ATOM | 3995 | CB | LEU | A | 509 | 68.299 | 67.893 | −22.320 | 1.00 | 9.95 | A |
| ATOM | 3996 | CG | LEU | A | 509 | 67.025 | 67.078 | −22.584 | 1.00 | 8.90 | A |
| ATOM | 3997 | CD1 | LEU | A | 509 | 65.900 | 68.010 | −23.009 | 1.00 | 8.36 | A |
| ATOM | 3998 | CD2 | LEU | A | 509 | 67.289 | 66.029 | −23.659 | 1.00 | 10.74 | A |
| ATOM | 3999 | C | LEU | A | 509 | 70.763 | 67.910 | −21.897 | 1.00 | 11.19 | A |
| ATOM | 4000 | O | LEU | A | 509 | 70.928 | 68.652 | −22.863 | 1.00 | 11.27 | A |
| ATOM | 4001 | N | THR | A | 510 | 71.673 | 67.781 | −20.938 | 1.00 | 11.02 | A |
| ATOM | 4002 | CA | THR | A | 510 | 72.930 | 68.516 | −21.011 | 1.00 | 11.45 | A |
| ATOM | 4003 | CB | THR | A | 510 | 73.089 | 69.481 | −19.814 | 1.00 | 11.70 | A |
| ATOM | 4004 | OG1 | THR | A | 510 | 72.005 | 70.417 | −19.815 | 1.00 | 10.96 | A |
| ATOM | 4005 | CG2 | THR | A | 510 | 74.408 | 70.253 | −19.904 | 1.00 | 11.60 | A |
| ATOM | 4006 | C | THR | A | 510 | 74.092 | 67.533 | −21.029 | 1.00 | 12.46 | A |
| ATOM | 4007 | O | THR | A | 510 | 74.105 | 66.568 | −20.266 | 1.00 | 12.49 | A |
| ATOM | 4008 | N | LYS | A | 511 | 75.055 | 67.772 | −21.915 | 1.00 | 13.67 | A |
| ATOM | 4009 | CA | LYS | A | 511 | 76.217 | 66.897 | −22.012 | 1.00 | 15.49 | A |
| ATOM | 4010 | CB | LYS | A | 511 | 77.275 | 67.512 | −22.930 | 1.00 | 17.27 | A |
| ATOM | 4011 | CG | LYS | A | 511 | 78.509 | 66.643 | −23.110 | 1.00 | 20.21 | A |
| ATOM | 4012 | CD | LYS | A | 511 | 79.482 | 67.269 | −24.092 | 1.00 | 22.41 | A |
| ATOM | 4013 | CE | LYS | A | 511 | 80.704 | 66.388 | −24.290 | 1.00 | 24.44 | A |
| ATOM | 4014 | NZ | LYS | A | 511 | 81.639 | 66.957 | −25.300 | 1.00 | 26.86 | A |
| ATOM | 4015 | C | LYS | A | 511 | 76.774 | 66.718 | −20.608 | 1.00 | 15.51 | A |
| ATOM | 4016 | O | LYS | A | 511 | 77.049 | 67.692 | −19.908 | 1.00 | 14.86 | A |
| ATOM | 4017 | N | PRO | A | 512 | 76.943 | 65.460 | −20.175 | 1.00 | 16.19 | A |
| ATOM | 4018 | CD | PRO | A | 512 | 76.648 | 64.242 | −20.952 | 1.00 | 16.95 | A |
| ATOM | 4019 | CA | PRO | A | 512 | 77.457 | 65.111 | −18.849 | 1.00 | 16.40 | A |
| ATOM | 4020 | CB | PRO | A | 512 | 77.744 | 63.620 | −18.983 | 1.00 | 17.38 | A |
| ATOM | 4021 | CG | PRO | A | 512 | 76.652 | 63.172 | −19.886 | 1.00 | 17.83 | A |
| ATOM | 4022 | C | PRO | A | 512 | 78.669 | 65.890 | −18.347 | 1.00 | 16.31 | A |
| ATOM | 4023 | O | PRO | A | 512 | 78.664 | 66.387 | −17.223 | 1.00 | 16.51 | A |
| ATOM | 4024 | N | SER | A | 513 | 79.701 | 66.000 | −19.177 | 1.00 | 16.07 | A |
| ATOM | 4025 | CA | SER | A | 513 | 80.920 | 66.701 | −18.778 | 1.00 | 16.40 | A |
| ATOM | 4026 | CB | SER | A | 513 | 82.074 | 66.309 | −19.708 | 1.00 | 16.21 | A |
| ATOM | 4027 | OG | SER | A | 513 | 81.769 | 66.605 | −21.058 | 1.00 | 17.37 | A |
| ATOM | 4028 | C | SER | A | 513 | 80.796 | 68.221 | −18.727 | 1.00 | 16.15 | A |
| ATOM | 4029 | O | SER | A | 513 | 81.738 | 68.910 | −18.335 | 1.00 | 16.45 | A |
| ATOM | 4030 | N | ILE | A | 514 | 79.637 | 68.740 | −19.121 | 1.00 | 15.99 | A |
| ATOM | 4031 | CA | ILE | A | 514 | 79.391 | 70.179 | −19.110 | 1.00 | 15.87 | A |
| ATOM | 4032 | CB | ILE | A | 514 | 78.813 | 70.659 | −20.468 | 1.00 | 16.66 | A |
| ATOM | 4033 | CG2 | ILE | A | 514 | 78.339 | 72.106 | −20.355 | 1.00 | 17.47 | A |
| ATOM | 4034 | CG1 | ILE | A | 514 | 79.874 | 70.512 | −21.563 | 1.00 | 17.94 | A |
| ATOM | 4035 | CD1 | ILE | A | 514 | 79.427 | 70.989 | −22.933 | 1.00 | 19.29 | A |
| ATOM | 4036 | C | ILE | A | 514 | 78.410 | 70.538 | −17.993 | 1.00 | 15.13 | A |
| ATOM | 4037 | O | ILE | A | 514 | 78.466 | 71.630 | −17.428 | 1.00 | 14.53 | A |
| ATOM | 4038 | N | TYR | A | 515 | 77.522 | 69.601 | −17.679 | 1.00 | 14.27 | A |
| ATOM | 4039 | CA | TYR | A | 515 | 76.510 | 69.778 | −16.638 | 1.00 | 13.78 | A |
| ATOM | 4040 | CB | TYR | A | 515 | 75.831 | 68.428 | −16.389 | 1.00 | 13.76 | A |
| ATOM | 4041 | CG | TYR | A | 515 | 74.793 | 68.410 | −15.295 | 1.00 | 13.28 | A |
| ATOM | 4042 | CD1 | TYR | A | 515 | 73.628 | 69.168 | −15.392 | 1.00 | 12.14 | A |
| ATOM | 4043 | CE1 | TYR | A | 515 | 72.649 | 69.112 | −14.395 | 1.00 | 12.51 | A |
| ATOM | 4044 | CD2 | TYR | A | 515 | 74.961 | 67.598 | −14.177 | 1.00 | 13.33 | A |
| ATOM | 4045 | CE2 | TYR | A | 515 | 73.999 | 67.534 | −13.182 | 1.00 | 12.19 | A |
| ATOM | 4046 | CZ | TYR | A | 515 | 72.846 | 68.290 | −13.295 | 1.00 | 12.93 | A |
| ATOM | 4047 | OH | TYR | A | 515 | 71.893 | 68.211 | −12.308 | 1.00 | 13.77 | A |
| ATOM | 4048 | C | TYR | A | 515 | 77.104 | 70.331 | −15.338 | 1.00 | 13.79 | A |
| ATOM | 4049 | O | TYR | A | 515 | 77.937 | 69.687 | −14.703 | 1.00 | 13.97 | A |
| ATOM | 4050 | N | SER | A | 516 | 76.669 | 71.528 | −14.951 | 1.00 | 14.47 | A |
| ATOM | 4051 | CA | SER | A | 516 | 77.152 | 72.183 | −13.733 | 1.00 | 15.55 | A |
| ATOM | 4052 | CB | SER | A | 516 | 78.192 | 73.248 | −14.091 | 1.00 | 16.76 | A |
| ATOM | 4053 | OG | SER | A | 516 | 78.809 | 73.768 | −12.928 | 1.00 | 18.55 | A |
| ATOM | 4054 | C | SER | A | 516 | 75.948 | 72.833 | −13.059 | 1.00 | 16.14 | A |
| ATOM | 4055 | O | SER | A | 516 | 75.719 | 74.037 | −13.184 | 1.00 | 16.36 | A |
| ATOM | 4056 | N | PRO | A | 517 | 75.166 | 72.037 | −12.315 | 1.00 | 16.21 | A |
| ATOM | 4057 | CD | PRO | A | 517 | 75.323 | 70.583 | −12.117 | 1.00 | 16.72 | A |
| ATOM | 4058 | CA | PRO | A | 517 | 73.971 | 72.522 | −11.630 | 1.00 | 16.81 | A |
| ATOM | 4059 | CB | PRO | A | 517 | 73.208 | 71.234 | −11.345 | 1.00 | 16.87 | A |
| ATOM | 4060 | CG | PRO | A | 517 | 74.317 | 70.294 | −11.013 | 1.00 | 16.68 | A |
| ATOM | 4061 | C | PRO | A | 517 | 74.067 | 73.387 | −10.382 | 1.00 | 17.01 | A |
| ATOM | 4062 | O | PRO | A | 517 | 74.914 | 73.182 | −9.513 | 1.00 | 18.50 | A |
| ATOM | 4063 | N | ASP | A | 518 | 73.169 | 74.364 | −10.331 | 1.00 | 17.32 | A |
| ATOM | 4064 | CA | ASP | A | 518 | 72.998 | 75.251 | −9.191 | 1.00 | 17.65 | A |
| ATOM | 4065 | CB | ASP | A | 518 | 72.942 | 76.716 | −9.619 | 1.00 | 18.97 | A |
| ATOM | 4066 | CG | ASP | A | 518 | 72.657 | 77.650 | −8.454 | 1.00 | 20.40 | A |
| ATOM | 4067 | OD1 | ASP | A | 518 | 72.026 | 77.206 | −7.469 | 1.00 | 20.68 | A |
| ATOM | 4068 | OD2 | ASP | A | 518 | 73.048 | 78.833 | −8.523 | 1.00 | 21.51 | A |
| ATOM | 4069 | C | ASP | A | 518 | 71.597 | 74.788 | −8.807 | 1.00 | 16.83 | A |
| ATOM | 4070 | O | ASP | A | 518 | 70.624 | 75.139 | −9.471 | 1.00 | 16.54 | A |
| ATOM | 4071 | N | PHE | A | 519 | 71.495 | 73.982 | −7.757 | 1.00 | 16.46 | A |
| ATOM | 4072 | CA | PHE | A | 519 | 70.205 | 73.440 | −7.348 | 1.00 | 16.01 | A |
| ATOM | 4073 | CB | PHE | A | 519 | 70.415 | 72.389 | −6.256 | 1.00 | 15.82 | A |
| ATOM | 4074 | CG | PHE | A | 519 | 71.267 | 71.229 | −6.697 | 1.00 | 15.47 | A |
| ATOM | 4075 | CD1 | PHE | A | 519 | 70.980 | 70.553 | −7.881 | 1.00 | 15.02 | A |
| ATOM | 4076 | CD2 | PHE | A | 519 | 72.352 | 70.811 | −5.932 | 1.00 | 15.53 | A |
| ATOM | 4077 | CE1 | PHE | A | 519 | 71.759 | 69.481 | −8.298 | 1.00 | 14.89 | A |
| ATOM | 4078 | CE2 | PHE | A | 519 | 73.138 | 69.736 | −6.341 | 1.00 | 15.39 | A |
| ATOM | 4079 | CZ | PHE | A | 519 | 72.841 | 69.071 | −7.525 | 1.00 | 15.53 | A |
| ATOM | 4080 | C | PHE | A | 519 | 69.136 | 74.442 | −6.925 | 1.00 | 16.61 | A |
| ATOM | 4081 | O | PHE | A | 519 | 68.004 | 74.057 | −6.638 | 1.00 | 16.65 | A |
| ATOM | 4082 | N | SER | A | 520 | 69.479 | 75.724 | −6.898 | 1.00 | 17.10 | A |
| ATOM | 4083 | CA | SER | A | 520 | 68.510 | 76.751 | −6.523 | 1.00 | 17.76 | A |
| ATOM | 4084 | CB | SER | A | 520 | 69.097 | 77.672 | −5.453 | 1.00 | 18.35 | A |
| ATOM | 4085 | OG | SER | A | 520 | 70.142 | 78.465 | −5.993 | 1.00 | 20.78 | A |
| ATOM | 4086 | C | SER | A | 520 | 68.138 | 77.588 | −7.744 | 1.00 | 17.75 | A |
| ATOM | 4087 | O | SER | A | 520 | 67.238 | 78.427 | −7.683 | 1.00 | 18.10 | A |
| ATOM | 4088 | N | PHE | A | 521 | 68.829 | 77.340 | −8.852 | 1.00 | 17.53 | A |
| ATOM | 4089 | CA | PHE | A | 521 | 68.620 | 78.085 | −10.088 | 1.00 | 17.29 | A |
| ATOM | 4090 | CB | PHE | A | 521 | 69.929 | 78.114 | −10.885 | 1.00 | 17.61 | A |
| ATOM | 4091 | CG | PHE | A | 521 | 69.910 | 79.058 | −12.053 | 1.00 | 18.36 | A |
| ATOM | 4092 | CD1 | PHE | A | 521 | 69.988 | 80.433 | −11.854 | 1.00 | 19.34 | A |
| ATOM | 4093 | CD2 | PHE | A | 521 | 69.797 | 78.574 | −13.353 | 1.00 | 18.76 | A |
| ATOM | 4094 | CE1 | PHE | A | 521 | 69.953 | 81.313 | −12.933 | 1.00 | 19.83 | A |
| ATOM | 4095 | CE2 | PHE | A | 521 | 69.762 | 79.445 | −14.438 | 1.00 | 19.30 | A |
| ATOM | 4096 | CZ | PHE | A | 521 | 69.839 | 80.818 | −14.228 | 1.00 | 19.85 | A |
| ATOM | 4097 | C | PHE | A | 521 | 67.504 | 77.534 | −10.974 | 1.00 | 16.99 | A |
| ATOM | 4098 | O | PHE | A | 521 | 67.261 | 76.328 | −11.012 | 1.00 | 17.26 | A |
| ATOM | 4099 | N | SER | A | 522 | 66.833 | 78.434 | −11.689 | 1.00 | 16.98 | A |
| ATOM | 4100 | CA | SER | A | 522 | 65.759 | 78.052 | −12.598 | 1.00 | 16.45 | A |
| ATOM | 4101 | CB | SER | A | 522 | 64.590 | 79.036 | −12.496 | 1.00 | 18.00 | A |
| ATOM | 4102 | OG | SER | A | 522 | 63.937 | 78.923 | −11.243 | 1.00 | 21.58 | A |
| ATOM | 4103 | C | SER | A | 522 | 66.282 | 78.028 | −14.033 | 1.00 | 14.76 | A |
| ATOM | 4104 | O | SER | A | 522 | 66.362 | 79.066 | −14.695 | 1.00 | 15.51 | A |
| ATOM | 4105 | N | TYR | A | 523 | 66.646 | 76.839 | −14.503 | 1.00 | 13.04 | A |
| ATOM | 4106 | CA | TYR | A | 523 | 67.155 | 76.676 | −15.860 | 1.00 | 12.11 | A |
| ATOM | 4107 | CB | TYR | A | 523 | 67.879 | 75.338 | −16.003 | 1.00 | 12.66 | A |
| ATOM | 4108 | CG | TYR | A | 523 | 69.152 | 75.276 | −15.205 | 1.00 | 12.36 | A |
| ATOM | 4109 | CD1 | TYR | A | 523 | 69.172 | 74.722 | −13.926 | 1.00 | 11.48 | A |
| ATOM | 4110 | CE1 | TYR | A | 523 | 70.337 | 74.718 | −13.167 | 1.00 | 13.82 | A |
| ATOM | 4111 | CD2 | TYR | A | 523 | 70.331 | 75.824 | −15.707 | 1.00 | 12.94 | A |
| ATOM | 4112 | CE2 | TYR | A | 523 | 71.497 | 75.828 | −14.957 | 1.00 | 13.68 | A |
| ATOM | 4113 | CZ | TYR | A | 523 | 71.493 | 75.275 | −13.688 | 1.00 | 14.52 | A |
| ATOM | 4114 | OH | TYR | A | 523 | 72.642 | 75.297 | −12.932 | 1.00 | 15.63 | A |
| ATOM | 4115 | C | TYR | A | 523 | 66.028 | 76.753 | −16.870 | 1.00 | 12.04 | A |
| ATOM | 4116 | O | TYR | A | 523 | 66.232 | 77.175 | −18.009 | 1.00 | 11.26 | A |
| ATOM | 4117 | N | PHE | A | 524 | 64.840 | 76.332 | −16.449 | 1.00 | 11.79 | A |
| ATOM | 4118 | CA | PHE | A | 524 | 63.664 | 76.371 | −17.306 | 1.00 | 11.61 | A |
| ATOM | 4119 | CB | PHE | A | 524 | 63.287 | 74.984 | −17.836 | 1.00 | 11.49 | A |
| ATOM | 4120 | CG | PHE | A | 524 | 64.319 | 74.353 | −18.715 | 1.00 | 10.85 | A |
| ATOM | 4121 | CD1 | PHE | A | 524 | 65.356 | 73.609 | −18.166 | 1.00 | 11.04 | A |
| ATOM | 4122 | CD2 | PHE | A | 524 | 64.241 | 74.482 | −20.098 | 1.00 | 11.26 | A |
| ATOM | 4123 | CE1 | PHE | A | 524 | 66.300 | 72.998 | −18.982 | 1.00 | 11.78 | A |
| ATOM | 4124 | CE2 | PHE | A | 524 | 65.179 | 73.876 | −20.924 | 1.00 | 10.39 | A |
| ATOM | 4125 | CZ | PHE | A | 524 | 66.211 | 73.132 | −20.364 | 1.00 | 11.02 | A |
| ATOM | 4126 | C | PHE | A | 524 | 62.466 | 76.873 | −16.528 | 1.00 | 13.17 | A |
| ATOM | 4127 | O | PHE | A | 524 | 62.380 | 76.701 | −15.311 | 1.00 | 13.07 | A |
| ATOM | 4128 | N | THR | A | 525 | 61.540 | 77.479 | −17.255 | 1.00 | 14.04 | A |
| ATOM | 4129 | CA | THR | A | 525 | 60.298 | 77.967 | −16.686 | 1.00 | 15.95 | A |
| ATOM | 4130 | CB | THR | A | 525 | 60.069 | 79.455 | −17.014 | 1.00 | 17.37 | A |
| ATOM | 4131 | OG1 | THR | A | 525 | 61.109 | 80.242 | −16.417 | 1.00 | 21.08 | A |
| ATOM | 4132 | CG2 | THR | A | 525 | 58.725 | 79.917 | −16.480 | 1.00 | 20.26 | A |
| ATOM | 4133 | C | THR | A | 525 | 59.206 | 77.138 | −17.356 | 1.00 | 14.82 | A |
| ATOM | 4134 | O | THR | A | 525 | 59.222 | 76.953 | −18.575 | 1.00 | 14.60 | A |
| ATOM | 4135 | N | LEU | A | 526 | 58.276 | 76.616 | −16.565 | 1.00 | 14.08 | A |
| ATOM | 4136 | CA | LEU | A | 526 | 57.184 | 75.830 | −17.122 | 1.00 | 14.04 | A |
| ATOM | 4137 | CB | LEU | A | 526 | 56.468 | 75.030 | −16.029 | 1.00 | 14.91 | A |
| ATOM | 4138 | CG | LEU | A | 526 | 56.977 | 73.638 | −15.661 | 1.00 | 16.17 | A |
| ATOM | 4139 | CD1 | LEU | A | 526 | 56.169 | 73.104 | −14.483 | 1.00 | 17.46 | A |
| ATOM | 4140 | CD2 | LEU | A | 526 | 56.844 | 72.710 | −16.858 | 1.00 | 16.37 | A |
| ATOM | 4141 | C | LEU | A | 526 | 56.176 | 76.754 | −17.785 | 1.00 | 13.66 | A |
| ATOM | 4142 | O | LEU | A | 526 | 55.864 | 77.825 | −17.265 | 1.00 | 15.13 | A |
| ATOM | 4143 | N | ASP | A | 527 | 55.675 | 76.339 | −18.941 | 1.00 | 12.47 | A |
| ATOM | 4144 | CA | ASP | A | 527 | 54.671 | 77.113 | −19.644 | 1.00 | 12.88 | A |
| ATOM | 4145 | CB | ASP | A | 527 | 55.142 | 77.470 | −21.055 | 1.00 | 13.45 | A |
| ATOM | 4146 | CG | ASP | A | 527 | 54.139 | 78.332 | −21.797 | 1.00 | 15.23 | A |
| ATOM | 4147 | OD1 | ASP | A | 527 | 53.873 | 79.462 | −21.335 | 1.00 | 15.10 | A |
| ATOM | 4148 | OD2 | ASP | A | 527 | 53.612 | 77.881 | −22.837 | 1.00 | 16.86 | A |
| ATOM | 4149 | C | ASP | A | 527 | 53.444 | 76.221 | −19.715 | 1.00 | 12.90 | A |
| ATOM | 4150 | O | ASP | A | 527 | 53.486 | 75.152 | −20.315 | 1.00 | 14.82 | A |
| ATOM | 4151 | N | ASP | A | 528 | 52.363 | 76.651 | −19.079 | 1.00 | 12.04 | A |
| ATOM | 4152 | CA | ASP | A | 528 | 51.126 | 75.879 | −19.064 | 1.00 | 11.43 | A |
| ATOM | 4153 | CB | ASP | A | 528 | 50.757 | 75.525 | −17.622 | 1.00 | 11.67 | A |
| ATOM | 4154 | CG | ASP | A | 528 | 49.732 | 74.413 | −17.538 | 1.00 | 11.75 | A |
| ATOM | 4155 | OD1 | ASP | A | 528 | 48.723 | 74.470 | −18.271 | 1.00 | 11.28 | A |
| ATOM | 4156 | OD2 | ASP | A | 528 | 49.935 | 73.486 | −16.729 | 1.00 | 11.32 | A |
| ATOM | 4157 | C | ASP | A | 528 | 50.039 | 76.744 | −19.687 | 1.00 | 11.92 | A |
| ATOM | 4158 | O | ASP | A | 528 | 49.701 | 77.793 | −19.157 | 1.00 | 10.88 | A |
| ATOM | 4159 | N | SER | A | 529 | 49.489 | 76.300 | −20.811 | 1.00 | 12.60 | A |
| ATOM | 4160 | CA | SER | A | 529 | 48.468 | 77.083 | −21.491 | 1.00 | 13.59 | A |
| ATOM | 4161 | CB | SER | A | 529 | 48.470 | 76.759 | −22.988 | 1.00 | 15.60 | A |
| ATOM | 4162 | OG | SER | A | 529 | 48.039 | 75.431 | −23.218 | 1.00 | 20.83 | A |
| ATOM | 4163 | C | SER | A | 529 | 47.057 | 76.912 | −20.940 | 1.00 | 12.92 | A |
| ATOM | 4164 | O | SER | A | 529 | 46.164 | 77.682 | −21.287 | 1.00 | 14.25 | A |
| ATOM | 4165 | N | ARG | A | 530 | 46.843 | 75.926 | −20.074 | 1.00 | 11.90 | A |
| ATOM | 4166 | CA | ARG | A | 530 | 45.501 | 75.711 | −19.549 | 1.00 | 12.42 | A |
| ATOM | 4167 | CB | ARG | A | 530 | 44.964 | 74.373 | −20.060 | 1.00 | 11.67 | A |
| ATOM | 4168 | CG | ARG | A | 530 | 44.887 | 74.353 | −21.581 | 1.00 | 12.07 | A |
| ATOM | 4169 | CD | ARG | A | 530 | 44.248 | 73.092 | −22.123 | 1.00 | 12.73 | A |
| ATOM | 4170 | NE | ARG | A | 530 | 44.981 | 71.897 | −21.729 | 1.00 | 12.73 | A |
| ATOM | 4171 | CZ | ARG | A | 530 | 44.758 | 70.691 | −22.240 | 1.00 | 13.69 | A |
| ATOM | 4172 | NH1 | ARG | A | 530 | 43.821 | 70.527 | −23.164 | 1.00 | 13.80 | A |
| ATOM | 4173 | NH2 | ARG | A | 530 | 45.476 | 69.655 | −21.832 | 1.00 | 14.84 | A |
| ATOM | 4174 | C | ARG | A | 530 | 45.327 | 75.825 | −18.041 | 1.00 | 12.87 | A |
| ATOM | 4175 | O | ARG | A | 530 | 44.268 | 75.496 | −17.507 | 1.00 | 14.42 | A |
| ATOM | 4176 | N | TRP | A | 531 | 46.365 | 76.285 | −17.354 | 1.00 | 12.16 | A |
| ATOM | 4177 | CA | TRP | A | 531 | 46.272 | 76.496 | −15.915 | 1.00 | 12.56 | A |
| ATOM | 4178 | CB | TRP | A | 531 | 46.546 | 75.221 | −15.115 | 1.00 | 13.21 | A |
| ATOM | 4179 | CG | TRP | A | 531 | 46.479 | 75.516 | −13.647 | 1.00 | 13.89 | A |
| ATOM | 4180 | CD2 | TRP | A | 531 | 45.292 | 75.619 | −12.853 | 1.00 | 15.00 | A |
| ATOM | 4181 | CE2 | TRP | A | 531 | 45.683 | 76.062 | −11.570 | 1.00 | 14.42 | A |
| ATOM | 4182 | CE3 | TRP | A | 531 | 43.934 | 75.385 | −13.103 | 1.00 | 14.98 | A |
| ATOM | 4183 | CD1 | TRP | A | 531 | 47.516 | 75.883 | −12.831 | 1.00 | 14.62 | A |
| ATOM | 4184 | NE1 | TRP | A | 531 | 47.045 | 76.214 | −11.585 | 1.00 | 16.00 | A |
| ATOM | 4185 | CZ2 | TRP | A | 531 | 44.763 | 76.275 | −10.537 | 1.00 | 15.89 | A |
| ATOM | 4186 | CZ3 | TRP | A | 531 | 43.019 | 75.598 | −12.078 | 1.00 | 15.43 | A |
| ATOM | 4187 | CH2 | TRP | A | 531 | 43.440 | 76.039 | −10.811 | 1.00 | 15.65 | A |
| ATOM | 4188 | C | TRP | A | 531 | 47.239 | 77.578 | −15.454 | 1.00 | 13.17 | A |
| ATOM | 4189 | O | TRP | A | 531 | 48.436 | 77.495 | −15.708 | 1.00 | 13.12 | A |
| ATOM | 4190 | N | PRO | A | 532 | 46.728 | 78.603 | −14.753 | 1.00 | 13.97 | A |
| ATOM | 4191 | CD | PRO | A | 532 | 47.581 | 79.550 | −14.016 | 1.00 | 14.36 | A |
| ATOM | 4192 | CA | PRO | A | 532 | 45.319 | 78.790 | −14.388 | 1.00 | 14.97 | A |
| ATOM | 4193 | CB | PRO | A | 532 | 45.360 | 80.011 | −13.472 | 1.00 | 14.79 | A |
| ATOM | 4194 | CG | PRO | A | 532 | 46.709 | 79.897 | −12.832 | 1.00 | 14.98 | A |
| ATOM | 4195 | C | PRO | A | 532 | 44.433 | 79.009 | −15.610 | 1.00 | 16.32 | A |
| ATOM | 4196 | O | PRO | A | 532 | 43.214 | 78.863 | −15.538 | 1.00 | 16.43 | A |
| ATOM | 4197 | N | GLY | A | 533 | 45.058 | 79.363 | −16.729 | 1.00 | 18.09 | A |
| ATOM | 4198 | CA | GLY | A | 533 | 44.313 | 79.573 | −17.956 | 1.00 | 20.35 | A |
| ATOM | 4199 | C | GLY | A | 533 | 44.110 | 81.021 | −18.355 | 1.00 | 22.43 | A |
| ATOM | 4200 | O | GLY | A | 533 | 44.136 | 81.924 | −17.517 | 1.00 | 21.90 | A |
| ATOM | 4201 | N | SER | A | 534 | 43.908 | 81.236 | −19.650 | 1.00 | 24.34 | A |
| ATOM | 4202 | CA | SER | A | 534 | 43.684 | 82.574 | −20.183 | 1.00 | 26.30 | A |
| ATOM | 4203 | CB | SER | A | 534 | 43.577 | 82.517 | −21.709 | 1.00 | 27.62 | A |
| ATOM | 4204 | OG | SER | A | 534 | 43.326 | 83.801 | −22.252 | 1.00 | 29.22 | A |
| ATOM | 4205 | C | SER | A | 534 | 42.396 | 83.135 | −19.593 | 1.00 | 26.95 | A |
| ATOM | 4206 | O | SER | A | 534 | 41.366 | 82.461 | −19.579 | 1.00 | 27.04 | A |
| ATOM | 4207 | N | GLY | A | 535 | 42.458 | 84.369 | −19.104 | 1.00 | 27.48 | A |
| ATOM | 4208 | CA | GLY | A | 535 | 41.282 | 84.985 | −18.516 | 1.00 | 28.19 | A |
| ATOM | 4209 | C | GLY | A | 535 | 41.168 | 84.676 | −17.036 | 1.00 | 28.77 | A |
| ATOM | 4210 | O | GLY | A | 535 | 40.300 | 85.208 | −16.342 | 1.00 | 28.75 | A |
| ATOM | 4211 | N | VAL | A | 536 | 42.047 | 83.805 | −16.551 | 1.00 | 28.86 | A |
| ATOM | 4212 | CA | VAL | A | 536 | 42.054 | 83.429 | −15.144 | 1.00 | 29.39 | A |
| ATOM | 4213 | CB | VAL | A | 536 | 42.205 | 81.900 | −14.973 | 1.00 | 29.14 | A |
| ATOM | 4214 | CG1 | VAL | A | 536 | 42.127 | 81.526 | −13.500 | 1.00 | 28.72 | A |
| ATOM | 4215 | CG2 | VAL | A | 536 | 41.120 | 81.183 | −15.759 | 1.00 | 28.88 | A |
| ATOM | 4216 | C | VAL | A | 536 | 43.222 | 84.126 | −14.457 | 1.00 | 30.17 | A |
| ATOM | 4217 | O | VAL | A | 536 | 43.062 | 84.745 | −13.405 | 1.00 | 29.60 | A |
| ATOM | 4218 | N | GLU | A | 537 | 44.399 | 84.026 | −15.065 | 1.00 | 31.25 | A |
| ATOM | 4219 | CA | GLU | A | 537 | 45.595 | 84.651 | −14.517 | 1.00 | 32.73 | A |
| ATOM | 4220 | CB | GLU | A | 537 | 46.093 | 83.859 | −13.303 | 1.00 | 33.69 | A |
| ATOM | 4221 | CG | GLU | A | 537 | 47.378 | 84.400 | −12.690 | 1.00 | 35.24 | A |
| ATOM | 4222 | CD | GLU | A | 537 | 47.769 | 83.681 | −11.411 | 1.00 | 36.16 | A |
| ATOM | 4223 | OE1 | GLU | A | 537 | 48.825 | 84.023 | −10.836 | 1.00 | 36.68 | A |
| ATOM | 4224 | OE2 | GLU | A | 537 | 47.023 | 82.777 | −10.977 | 1.00 | 36.57 | A |
| ATOM | 4225 | C | GLU | A | 537 | 46.702 | 84.738 | −15.560 | 1.00 | 33.20 | A |
| ATOM | 4226 | O | GLU | A | 537 | 47.132 | 83.720 | −16.101 | 1.00 | 33.31 | A |
| ATOM | 4227 | N | ASP | A | 538 | 47.156 | 85.953 | −15.852 | 1.00 | 33.93 | A |
| ATOM | 4228 | CA | ASP | A | 538 | 48.236 | 86.131 | −16.816 | 1.00 | 34.52 | A |
| ATOM | 4229 | CB | ASP | A | 538 | 48.368 | 87.600 | −17.225 | 1.00 | 36.03 | A |
| ATOM | 4230 | CG | ASP | A | 538 | 49.455 | 87.816 | −18.263 | 1.00 | 37.32 | A |
| ATOM | 4231 | OD1 | ASP | A | 538 | 49.350 | 87.232 | −19.362 | 1.00 | 37.91 | A |
| ATOM | 4232 | OD2 | ASP | A | 538 | 50.414 | 88.565 | −17.979 | 1.00 | 38.51 | A |
| ATOM | 4233 | C | ASP | A | 538 | 49.500 | 85.677 | −16.103 | 1.00 | 33.98 | A |
| ATOM | 4234 | O | ASP | A | 538 | 50.185 | 86.474 | −15.462 | 1.00 | 34.26 | A |
| ATOM | 4235 | N | SER | A | 539 | 49.798 | 84.388 | −16.212 | 1.00 | 32.86 | A |
| ATOM | 4236 | CA | SER | A | 539 | 50.958 | 83.813 | −15.547 | 1.00 | 31.79 | A |
| ATOM | 4237 | CB | SER | A | 539 | 50.523 | 82.598 | −14.725 | 1.00 | 31.81 | A |
| ATOM | 4238 | OG | SER | A | 539 | 49.889 | 81.634 | −15.551 | 1.00 | 32.64 | A |
| ATOM | 4239 | C | SER | A | 539 | 52.093 | 83.405 | −16.477 | 1.00 | 30.65 | A |
| ATOM | 4240 | O | SER | A | 539 | 53.264 | 83.579 | −16.142 | 1.00 | 30.92 | A |
| ATOM | 4241 | N | ARG | A | 540 | 51.755 | 82.858 | −17.640 | 1.00 | 29.09 | A |
| ATOM | 4242 | CA | ARG | A | 540 | 52.784 | 82.416 | −18.571 | 1.00 | 26.85 | A |
| ATOM | 4243 | CB | ARG | A | 540 | 52.164 | 81.572 | −19.691 | 1.00 | 26.27 | A |
| ATOM | 4244 | CG | ARG | A | 540 | 50.972 | 82.187 | −20.384 | 1.00 | 24.91 | A |
| ATOM | 4245 | CD | ARG | A | 540 | 50.198 | 81.126 | −21.168 | 1.00 | 21.40 | A |
| ATOM | 4246 | NE | ARG | A | 540 | 51.053 | 80.373 | −22.084 | 1.00 | 18.78 | A |
| ATOM | 4247 | CZ | ARG | A | 540 | 50.621 | 79.799 | −23.203 | 1.00 | 18.16 | A |
| ATOM | 4248 | NH1 | ARG | A | 540 | 49.343 | 79.892 | −23.549 | 1.00 | 17.98 | A |
| ATOM | 4249 | NH2 | ARG | A | 540 | 51.468 | 79.143 | −23.983 | 1.00 | 17.24 | A |
| ATOM | 4250 | C | ARG | A | 540 | 53.627 | 83.550 | −19.139 | 1.00 | 25.67 | A |
| ATOM | 4251 | O | ARG | A | 540 | 53.131 | 84.638 | −19.438 | 1.00 | 26.07 | A |
| ATOM | 4252 | N | THR | A | 541 | 54.919 | 83.275 | −19.269 | 1.00 | 23.91 | A |
| ATOM | 4253 | CA | THR | A | 541 | 55.877 | 84.246 | −19.767 | 1.00 | 22.62 | A |
| ATOM | 4254 | CB | THR | A | 541 | 57.299 | 83.885 | −19.316 | 1.00 | 23.68 | A |
| ATOM | 4255 | OG1 | THR | A | 541 | 57.696 | 82.662 | −19.945 | 1.00 | 24.78 | A |
| ATOM | 4256 | CG2 | THR | A | 541 | 57.352 | 83.702 | −17.808 | 1.00 | 23.48 | A |
| ATOM | 4257 | C | THR | A | 541 | 55.887 | 84.337 | −21.283 | 1.00 | 20.98 | A |
| ATOM | 4258 | O | THR | A | 541 | 55.492 | 83.405 | −21.982 | 1.00 | 21.78 | A |
| ATOM | 4259 | N | THR | A | 542 | 56.345 | 85.477 | −21.782 | 1.00 | 18.43 | A |
| ATOM | 4260 | CA | THR | A | 542 | 56.448 | 85.695 | −23.211 | 1.00 | 16.38 | A |
| ATOM | 4261 | CB | THR | A | 542 | 56.098 | 87.151 | −23.592 | 1.00 | 16.52 | A |
| ATOM | 4262 | OG1 | THR | A | 542 | 54.729 | 87.420 | −23.269 | 1.00 | 16.56 | A |
| ATOM | 4263 | CG2 | THR | A | 542 | 56.322 | 87.383 | −25.081 | 1.00 | 16.03 | A |
| ATOM | 4264 | C | THR | A | 542 | 57.900 | 85.445 | −23.577 | 1.00 | 14.95 | A |
| ATOM | 4265 | O | THR | A | 542 | 58.808 | 85.888 | −22.870 | 1.00 | 14.70 | A |
| ATOM | 4266 | N | ILE | A | 543 | 58.124 | 84.710 | −24.658 | 1.00 | 13.40 | A |
| ATOM | 4267 | CA | ILE | A | 543 | 59.482 | 84.463 | −25.110 | 1.00 | 12.55 | A |
| ATOM | 4268 | CB | ILE | A | 543 | 59.553 | 83.220 | −26.014 | 1.00 | 12.30 | A |
| ATOM | 4269 | CG2 | ILE | A | 543 | 60.943 | 83.095 | −26.627 | 1.00 | 12.36 | A |
| ATOM | 4270 | CG1 | ILE | A | 543 | 59.200 | 81.973 | −25.190 | 1.00 | 12.14 | A |
| ATOM | 4271 | CD1 | ILE | A | 543 | 59.126 | 80.694 | −25.997 | 1.00 | 11.44 | A |
| ATOM | 4272 | C | ILE | A | 543 | 59.838 | 85.723 | −25.893 | 1.00 | 13.00 | A |
| ATOM | 4273 | O | ILE | A | 543 | 59.273 | 85.990 | −26.954 | 1.00 | 13.68 | A |
| ATOM | 4274 | N | ILE | A | 544 | 60.753 | 86.510 | −25.340 | 1.00 | 13.31 | A |
| ATOM | 4275 | CA | ILE | A | 544 | 61.158 | 87.762 | −25.958 | 1.00 | 13.70 | A |
| ATOM | 4276 | CB | ILE | A | 544 | 61.448 | 88.825 | −24.875 | 1.00 | 14.36 | A |
| ATOM | 4277 | CG2 | ILE | A | 544 | 61.862 | 90.134 | −25.521 | 1.00 | 15.40 | A |
| ATOM | 4278 | CG1 | ILE | A | 544 | 60.192 | 89.038 | −24.022 | 1.00 | 15.13 | A |
| ATOM | 4279 | CD1 | ILE | A | 544 | 60.359 | 90.045 | −22.898 | 1.00 | 17.93 | A |
| ATOM | 4280 | C | ILE | A | 544 | 62.370 | 87.590 | −26.862 | 1.00 | 14.58 | A |
| ATOM | 4281 | O | ILE | A | 544 | 63.464 | 87.254 | −26.407 | 1.00 | 14.06 | A |
| ATOM | 4282 | N | LEU | A | 545 | 62.148 | 87.812 | −28.153 | 1.00 | 14.60 | A |
| ATOM | 4283 | CA | LEU | A | 545 | 63.195 | 87.691 | −29.157 | 1.00 | 14.96 | A |
| ATOM | 4284 | CB | LEU | A | 545 | 62.841 | 86.592 | −30.162 | 1.00 | 14.72 | A |
| ATOM | 4285 | CG | LEU | A | 545 | 62.557 | 85.197 | −29.596 | 1.00 | 14.29 | A |
| ATOM | 4286 | CD1 | LEU | A | 545 | 62.158 | 84.259 | −30.725 | 1.00 | 14.25 | A |
| ATOM | 4287 | CD2 | LEU | A | 545 | 63.792 | 84.672 | −28.877 | 1.00 | 13.93 | A |
| ATOM | 4288 | C | LEU | A | 545 | 63.345 | 89.019 | −29.887 | 1.00 | 15.87 | A |
| ATOM | 4289 | O | LEU | A | 545 | 62.393 | 89.791 | −29.988 | 1.00 | 16.34 | A |
| ATOM | 4290 | N | GLY | A | 546 | 64.544 | 89.275 | −30.395 | 1.00 | 16.68 | A |
| ATOM | 4291 | CA | GLY | A | 546 | 64.795 | 90.512 | −31.111 | 1.00 | 18.01 | A |
| ATOM | 4292 | C | GLY | A | 546 | 66.227 | 90.584 | −31.597 | 1.00 | 19.27 | A |
| ATOM | 4293 | O | GLY | A | 546 | 67.138 | 90.083 | −30.942 | 1.00 | 17.83 | A |
| ATOM | 4294 | N | GLU | A | 547 | 66.426 | 91.220 | −32.745 | 1.00 | 21.14 | A |
| ATOM | 4295 | CA | GLU | A | 547 | 67.754 | 91.359 | −33.334 | 1.00 | 23.92 | A |
| ATOM | 4296 | CB | GLU | A | 547 | 67.679 | 92.234 | −34.586 | 1.00 | 27.11 | A |
| ATOM | 4297 | CG | GLU | A | 547 | 66.677 | 91.771 | −35.625 | 1.00 | 32.07 | A |
| ATOM | 4298 | CD | GLU | A | 547 | 66.480 | 92.796 | −36.725 | 1.00 | 34.39 | A |
| ATOM | 4299 | OE1 | GLU | A | 547 | 67.457 | 93.089 | −37.449 | 1.00 | 35.80 | A |
| ATOM | 4300 | OE2 | GLU | A | 547 | 65.350 | 93.314 | −36.862 | 1.00 | 35.52 | A |
| ATOM | 4301 | C | GLU | A | 547 | 68.756 | 91.980 | −32.368 | 1.00 | 23.56 | A |
| ATOM | 4302 | O | GLU | A | 547 | 69.926 | 91.600 | −32.342 | 1.00 | 24.12 | A |
| ATOM | 4303 | N | ASP | A | 548 | 68.291 | 92.939 | −31.574 | 1.00 | 22.91 | A |
| ATOM | 4304 | CA | ASP | A | 548 | 69.159 | 93.634 | −30.630 | 1.00 | 22.77 | A |
| ATOM | 4305 | CB | ASP | A | 548 | 68.772 | 95.114 | −30.561 | 1.00 | 23.80 | A |
| ATOM | 4306 | CG | ASP | A | 548 | 68.956 | 95.830 | −31.884 | 1.00 | 24.78 | A |
| ATOM | 4307 | OD1 | ASP | A | 548 | 70.097 | 95.863 | −32.391 | 1.00 | 26.00 | A |
| ATOM | 4308 | OD2 | ASP | A | 548 | 67.959 | 96.363 | −32.416 | 1.00 | 25.77 | A |
| ATOM | 4309 | C | ASP | A | 548 | 69.159 | 93.058 | −29.220 | 1.00 | 22.13 | A |
| ATOM | 4310 | O | ASP | A | 548 | 69.669 | 93.692 | −28.295 | 1.00 | 22.21 | A |
| ATOM | 4311 | N | ILE | A | 549 | 68.600 | 91.866 | −29.039 | 1.00 | 20.62 | A |
| ATOM | 4312 | CA | ILE | A | 549 | 68.570 | 91.286 | −27.702 | 1.00 | 19.69 | A |
| ATOM | 4313 | CB | ILE | A | 549 | 67.253 | 91.667 | −26.967 | 1.00 | 20.73 | A |
| ATOM | 4314 | CG2 | ILE | A | 549 | 66.047 | 91.126 | −27.720 | 1.00 | 20.71 | A |
| ATOM | 4315 | CG1 | ILE | A | 549 | 67.280 | 91.132 | −25.535 | 1.00 | 21.62 | A |
| ATOM | 4316 | CD1 | ILE | A | 549 | 68.364 | 91.745 | −24.675 | 1.00 | 23.70 | A |
| ATOM | 4317 | C | ILE | A | 549 | 68.758 | 89.771 | −27.647 | 1.00 | 18.85 | A |
| ATOM | 4318 | O | ILE | A | 549 | 69.579 | 89.276 | −26.875 | 1.00 | 18.42 | A |
| ATOM | 4319 | N | LEU | A | 550 | 68.011 | 89.038 | −28.466 | 1.00 | 17.47 | A |
| ATOM | 4320 | CA | LEU | A | 550 | 68.104 | 87.580 | −28.473 | 1.00 | 16.89 | A |
| ATOM | 4321 | CB | LEU | A | 550 | 67.420 | 87.014 | −27.227 | 1.00 | 17.32 | A |
| ATOM | 4322 | CG | LEU | A | 550 | 67.461 | 85.495 | −27.058 | 1.00 | 17.26 | A |
| ATOM | 4323 | CD1 | LEU | A | 550 | 68.903 | 85.039 | −26.909 | 1.00 | 17.29 | A |
| ATOM | 4324 | CD2 | LEU | A | 550 | 66.649 | 85.096 | −25.838 | 1.00 | 17.14 | A |
| ATOM | 4325 | C | LEU | A | 550 | 67.444 | 87.015 | −29.724 | 1.00 | 16.51 | A |
| ATOM | 4326 | O | LEU | A | 550 | 66.238 | 87.160 | −29.920 | 1.00 | 16.78 | A |
| ATOM | 4327 | N | PRO | A | 551 | 68.226 | 86.351 | −30.587 | 1.00 | 16.11 | A |
| ATOM | 4328 | CD | PRO | A | 551 | 69.696 | 86.234 | −30.567 | 1.00 | 16.87 | A |
| ATOM | 4329 | CA | PRO | A | 551 | 67.675 | 85.783 | −31.819 | 1.00 | 15.76 | A |
| ATOM | 4330 | CB | PRO | A | 551 | 68.915 | 85.591 | −32.688 | 1.00 | 16.77 | A |
| ATOM | 4331 | CG | PRO | A | 551 | 69.962 | 85.248 | −31.690 | 1.00 | 18.35 | A |
| ATOM | 4332 | C | PRO | A | 551 | 66.849 | 84.502 | −31.705 | 1.00 | 14.86 | A |
| ATOM | 4333 | O | PRO | A | 551 | 65.944 | 84.279 | −32.509 | 1.00 | 15.49 | A |
| ATOM | 4334 | N | SER | A | 552 | 67.152 | 83.661 | −30.722 | 1.00 | 13.64 | A |
| ATOM | 4335 | CA | SER | A | 552 | 66.415 | 82.407 | −30.589 | 1.00 | 12.75 | A |
| ATOM | 4336 | CB | SER | A | 552 | 67.084 | 81.317 | −31.425 | 1.00 | 13.26 | A |
| ATOM | 4337 | OG | SER | A | 552 | 68.373 | 81.018 | −30.924 | 1.00 | 15.05 | A |
| ATOM | 4338 | C | SER | A | 552 | 66.276 | 81.919 | −29.156 | 1.00 | 11.84 | A |
| ATOM | 4339 | O | SER | A | 552 | 66.931 | 82.422 | −28.246 | 1.00 | 11.84 | A |
| ATOM | 4340 | N | LYS | A | 553 | 65.422 | 80.917 | −28.979 | 1.00 | 10.82 | A |
| ATOM | 4341 | CA | LYS | A | 553 | 65.153 | 80.350 | −27.667 | 1.00 | 11.24 | A |
| ATOM | 4342 | CB | LYS | A | 553 | 63.958 | 81.071 | −27.037 | 1.00 | 11.54 | A |
| ATOM | 4343 | CG | LYS | A | 553 | 63.441 | 80.440 | −25.742 | 1.00 | 12.80 | A |
| ATOM | 4344 | CD | LYS | A | 553 | 64.467 | 80.532 | −24.630 | 1.00 | 13.25 | A |
| ATOM | 4345 | CE | LYS | A | 553 | 64.703 | 81.972 | −24.200 | 1.00 | 13.22 | A |
| ATOM | 4346 | NZ | LYS | A | 553 | 65.871 | 82.085 | −23.281 | 1.00 | 14.05 | A |
| ATOM | 4347 | C | LYS | A | 553 | 64.843 | 78.861 | −27.746 | 1.00 | 10.65 | A |
| ATOM | 4348 | O | LYS | A | 553 | 64.058 | 78.422 | −28.588 | 1.00 | 10.51 | A |
| ATOM | 4349 | N | HIS | A | 554 | 65.464 | 78.088 | −26.863 | 1.00 | 10.75 | A |
| ATOM | 4350 | CA | HIS | A | 554 | 65.211 | 76.656 | −26.817 | 1.00 | 11.14 | A |
| ATOM | 4351 | CB | HIS | A | 554 | 66.426 | 75.894 | −26.282 | 1.00 | 12.98 | A |
| ATOM | 4352 | CG | HIS | A | 554 | 67.562 | 75.806 | −27.250 | 1.00 | 14.49 | A |
| ATOM | 4353 | CD2 | HIS | A | 554 | 68.093 | 76.726 | −28.088 | 1.00 | 16.00 | A |
| ATOM | 4354 | ND1 | HIS | A | 554 | 68.315 | 74.662 | −27.410 | 1.00 | 17.03 | A |
| ATOM | 4355 | CE1 | HIS | A | 554 | 69.261 | 74.883 | −28.305 | 1.00 | 14.97 | A |
| ATOM | 4356 | NE2 | HIS | A | 554 | 69.148 | 76.128 | −28.732 | 1.00 | 18.38 | A |
| ATOM | 4357 | C | HIS | A | 554 | 64.028 | 76.358 | −25.906 | 1.00 | 10.44 | A |
| ATOM | 4358 | O | HIS | A | 554 | 63.874 | 76.972 | −24.848 | 1.00 | 10.76 | A |
| ATOM | 4359 | N | VAL | A | 555 | 63.194 | 75.422 | −26.340 | 1.00 | 10.09 | A |
| ATOM | 4360 | CA | VAL | A | 555 | 62.049 | 74.971 | −25.562 | 1.00 | 9.37 | A |
| ATOM | 4361 | CB | VAL | A | 555 | 60.697 | 75.425 | −26.165 | 1.00 | 9.28 | A |
| ATOM | 4362 | CG1 | VAL | A | 555 | 60.560 | 76.934 | −26.045 | 1.00 | 9.53 | A |
| ATOM | 4363 | CG2 | VAL | A | 555 | 60.584 | 74.987 | −27.613 | 1.00 | 9.57 | A |
| ATOM | 4364 | C | VAL | A | 555 | 62.123 | 73.452 | −25.554 | 1.00 | 9.23 | A |
| ATOM | 4365 | O | VAL | A | 555 | 62.656 | 72.849 | −26.485 | 1.00 | 9.24 | A |
| ATOM | 4366 | N | VAL | A | 556 | 61.601 | 72.836 | −24.499 | 1.00 | 7.97 | A |
| ATOM | 4367 | CA | VAL | A | 556 | 61.629 | 71.385 | −24.368 | 1.00 | 7.89 | A |
| ATOM | 4368 | CB | VAL | A | 556 | 62.664 | 70.944 | −23.299 | 1.00 | 7.99 | A |
| ATOM | 4369 | CG1 | VAL | A | 556 | 62.584 | 69.440 | −23.078 | 1.00 | 7.88 | A |
| ATOM | 4370 | CG2 | VAL | A | 556 | 64.066 | 71.352 | −23.717 | 1.00 | 9.27 | A |
| ATOM | 4371 | C | VAL | A | 556 | 60.266 | 70.857 | −23.940 | 1.00 | 7.33 | A |
| ATOM | 4372 | O | VAL | A | 556 | 59.616 | 71.443 | −23.077 | 1.00 | 7.50 | A |
| ATOM | 4373 | N | MSE | A | 557 | 59.840 | 69.757 | −24.551 | 1.00 | 7.30 | A |
| ATOM | 4374 | CA | MSE | A | 557 | 58.571 | 69.130 | −24.197 | 1.00 | 8.38 | A |
| ATOM | 4375 | CB | MSE | A | 557 | 57.688 | 68.912 | −25.433 | 1.00 | 9.38 | A |
| ATOM | 4376 | CG | MSE | A | 557 | 56.636 | 69.984 | −25.676 | 1.00 | 12.19 | A |
| ATOM | 4377 | SE | MSE | A | 557 | 57.378 | 71.731 | −25.960 | 1.00 | 17.55 | A |
| ATOM | 4378 | CE | MSE | A | 557 | 58.381 | 71.378 | −27.570 | 1.00 | 13.47 | A |
| ATOM | 4379 | C | MSE | A | 557 | 58.824 | 67.782 | −23.543 | 1.00 | 8.15 | A |
| ATOM | 4380 | O | MSE | A | 557 | 59.712 | 67.037 | −23.955 | 1.00 | 8.86 | A |
| ATOM | 4381 | N | HIS | A | 558 | 58.040 | 67.482 | −22.515 | 1.00 | 7.51 | A |
| ATOM | 4382 | CA | HIS | A | 558 | 58.134 | 66.204 | −21.823 | 1.00 | 6.67 | A |
| ATOM | 4383 | CB | HIS | A | 558 | 58.318 | 66.401 | −20.318 | 1.00 | 7.78 | A |
| ATOM | 4384 | CG | HIS | A | 558 | 58.239 | 65.129 | −19.529 | 1.00 | 7.30 | A |
| ATOM | 4385 | CD2 | HIS | A | 558 | 58.985 | 63.999 | −19.577 | 1.00 | 7.61 | A |
| ATOM | 4386 | ND1 | HIS | A | 558 | 57.301 | 64.926 | −18.539 | 1.00 | 7.80 | A |
| ATOM | 4387 | CE1 | HIS | A | 558 | 57.473 | 63.727 | −18.012 | 1.00 | 7.28 | A |
| ATOM | 4388 | NE2 | HIS | A | 558 | 58.489 | 63.143 | −18.623 | 1.00 | 7.93 | A |
| ATOM | 4389 | C | HIS | A | 558 | 56.845 | 65.433 | −22.061 | 1.00 | 7.17 | A |
| ATOM | 4390 | O | HIS | A | 558 | 55.757 | 66.011 | −22.049 | 1.00 | 6.98 | A |
| ATOM | 4391 | N | ASN | A | 559 | 56.976 | 64.129 | −22.283 | 1.00 | 6.57 | A |
| ATOM | 4392 | CA | ASN | A | 559 | 55.832 | 63.253 | −22.511 | 1.00 | 6.63 | A |
| ATOM | 4393 | CB | ASN | A | 559 | 55.926 | 62.621 | −23.905 | 1.00 | 7.17 | A |
| ATOM | 4394 | CG | ASN | A | 559 | 54.910 | 61.516 | −24.119 | 1.00 | 7.78 | A |
| ATOM | 4395 | OD1 | ASN | A | 559 | 53.821 | 61.540 | −23.550 | 1.00 | 8.71 | A |
| ATOM | 4396 | ND2 | ASN | A | 559 | 55.261 | 60.542 | −24.960 | 1.00 | 8.27 | A |
| ATOM | 4397 | C | ASN | A | 559 | 55.857 | 62.165 | −21.443 | 1.00 | 6.46 | A |
| ATOM | 4398 | O | ASN | A | 559 | 56.619 | 61.210 | −21.545 | 1.00 | 7.32 | A |
| ATOM | 4399 | N | THR | A | 560 | 55.015 | 62.306 | −20.425 | 1.00 | 6.42 | A |
| ATOM | 4400 | CA | THR | A | 560 | 54.983 | 61.336 | −19.335 | 1.00 | 7.03 | A |
| ATOM | 4401 | CB | THR | A | 560 | 54.219 | 61.925 | −18.120 | 1.00 | 6.72 | A |
| ATOM | 4402 | OG1 | THR | A | 560 | 54.541 | 61.177 | −16.940 | 1.00 | 6.98 | A |
| ATOM | 4403 | CG2 | THR | A | 560 | 52.720 | 61.877 | −18.350 | 1.00 | 7.05 | A |
| ATOM | 4404 | C | THR | A | 560 | 54.390 | 59.969 | −19.714 | 1.00 | 7.51 | A |
| ATOM | 4405 | O | THR | A | 560 | 54.588 | 58.982 | −18.999 | 1.00 | 7.07 | A |
| ATOM | 4406 | N | LEU | A | 561 | 53.674 | 59.904 | −20.835 | 1.00 | 7.49 | A |
| ATOM | 4407 | CA | LEU | A | 561 | 53.065 | 58.646 | −21.273 | 1.00 | 7.71 | A |
| ATOM | 4408 | CB | LEU | A | 561 | 51.904 | 58.925 | −22.233 | 1.00 | 8.47 | A |
| ATOM | 4409 | CG | LEU | A | 561 | 50.776 | 59.818 | −21.701 | 1.00 | 9.12 | A |
| ATOM | 4410 | CD1 | LEU | A | 561 | 49.724 | 60.008 | −22.787 | 1.00 | 9.86 | A |
| ATOM | 4411 | CD2 | LEU | A | 561 | 50.147 | 59.187 | −20.462 | 1.00 | 7.91 | A |
| ATOM | 4412 | C | LEU | A | 561 | 54.089 | 57.728 | −21.949 | 1.00 | 7.77 | A |
| ATOM | 4413 | O | LEU | A | 561 | 54.993 | 58.196 | −22.649 | 1.00 | 8.16 | A |
| ATOM | 4414 | N | PRO | A | 562 | 53.943 | 56.404 | −21.765 | 1.00 | 8.25 | A |
| ATOM | 4415 | CD | PRO | A | 562 | 52.970 | 55.743 | −20.872 | 1.00 | 7.40 | A |
| ATOM | 4416 | CA | PRO | A | 562 | 54.862 | 55.419 | −22.346 | 1.00 | 8.17 | A |
| ATOM | 4417 | CB | PRO | A | 562 | 54.675 | 54.212 | −21.439 | 1.00 | 8.14 | A |
| ATOM | 4418 | CG | PRO | A | 562 | 53.202 | 54.266 | −21.158 | 1.00 | 8.36 | A |
| ATOM | 4419 | C | PRO | A | 562 | 54.705 | 55.064 | −23.821 | 1.00 | 8.36 | A |
| ATOM | 4420 | O | PRO | A | 562 | 54.825 | 53.902 | −24.205 | 1.00 | 9.39 | A |
| ATOM | 4421 | N | HIS | A | 563 | 54.422 | 56.058 | −24.649 | 1.00 | 8.47 | A |
| ATOM | 4422 | CA | HIS | A | 563 | 54.322 | 55.820 | −26.081 | 1.00 | 9.07 | A |
| ATOM | 4423 | CB | HIS | A | 563 | 52.926 | 55.308 | −26.493 | 1.00 | 9.42 | A |
| ATOM | 4424 | CG | HIS | A | 563 | 51.790 | 56.182 | −26.058 | 1.00 | 10.44 | A |
| ATOM | 4425 | CD2 | HIS | A | 563 | 51.289 | 57.326 | −26.582 | 1.00 | 9.83 | A |
| ATOM | 4426 | ND1 | HIS | A | 563 | 50.998 | 55.881 | −24.972 | 1.00 | 10.12 | A |
| ATOM | 4427 | CE1 | HIS | A | 563 | 50.056 | 56.799 | −24.846 | 1.00 | 10.52 | A |
| ATOM | 4428 | NE2 | HIS | A | 563 | 50.210 | 57.688 | −25.811 | 1.00 | 10.71 | A |
| ATOM | 4429 | C | HIS | A | 563 | 54.643 | 57.118 | −26.790 | 1.00 | 9.23 | A |
| ATOM | 4430 | O | HIS | A | 563 | 54.465 | 58.197 | −26.224 | 1.00 | 10.26 | A |
| ATOM | 4431 | N | TRP | A | 564 | 55.157 | 57.014 | −28.010 | 1.00 | 9.71 | A |
| ATOM | 4432 | CA | TRP | A | 564 | 55.470 | 58.210 | −28.775 | 1.00 | 9.67 | A |
| ATOM | 4433 | CB | TRP | A | 564 | 55.966 | 57.858 | −30.177 | 1.00 | 10.28 | A |
| ATOM | 4434 | CG | TRP | A | 564 | 57.389 | 57.435 | −30.214 | 1.00 | 10.78 | A |
| ATOM | 4435 | CD2 | TRP | A | 564 | 58.527 | 58.285 | −30.382 | 1.00 | 12.00 | A |
| ATOM | 4436 | CE2 | TRP | A | 564 | 59.674 | 57.461 | −30.328 | 1.00 | 11.44 | A |
| ATOM | 4437 | CE3 | TRP | A | 564 | 58.692 | 59.667 | −30.572 | 1.00 | 12.35 | A |
| ATOM | 4438 | CD1 | TRP | A | 564 | 57.872 | 56.169 | −30.069 | 1.00 | 11.14 | A |
| ATOM | 4439 | NE1 | TRP | A | 564 | 59.243 | 56.175 | −30.137 | 1.00 | 11.65 | A |
| ATOM | 4440 | CZ2 | TRP | A | 564 | 60.972 | 57.971 | −30.458 | 1.00 | 12.05 | A |
| ATOM | 4441 | CZ3 | TRP | A | 564 | 59.982 | 60.174 | −30.702 | 1.00 | 12.66 | A |
| ATOM | 4442 | CH2 | TRP | A | 564 | 61.105 | 59.326 | −30.645 | 1.00 | 13.34 | A |
| ATOM | 4443 | C | TRP | A | 564 | 54.198 | 59.018 | −28.889 | 1.00 | 9.59 | A |
| ATOM | 4444 | O | TRP | A | 564 | 53.116 | 58.466 | −29.107 | 1.00 | 10.25 | A |
| ATOM | 4445 | N | ARG | A | 565 | 54.314 | 60.327 | −28.725 | 1.00 | 9.71 | A |
| ATOM | 4446 | CA | ARG | A | 565 | 53.134 | 61.160 | −28.818 | 1.00 | 10.44 | A |
| ATOM | 4447 | CB | ARG | A | 565 | 52.547 | 61.423 | −27.418 | 1.00 | 10.22 | A |
| ATOM | 4448 | CG | ARG | A | 565 | 51.315 | 62.336 | −27.437 | 1.00 | 12.89 | A |
| ATOM | 4449 | CD | ARG | A | 565 | 50.491 | 62.296 | −26.146 | 1.00 | 12.46 | A |
| ATOM | 4450 | NE | ARG | A | 565 | 51.287 | 62.544 | −24.949 | 1.00 | 12.69 | A |
| ATOM | 4451 | CZ | ARG | A | 565 | 50.804 | 63.063 | −23.823 | 1.00 | 12.00 | A |
| ATOM | 4452 | NH1 | ARG | A | 565 | 49.521 | 63.401 | −23.736 | 1.00 | 11.76 | A |
| ATOM | 4453 | NH2 | ARG | A | 565 | 51.603 | 63.240 | −22.782 | 1.00 | 10.34 | A |
| ATOM | 4454 | C | ARG | A | 565 | 53.396 | 62.477 | −29.515 | 1.00 | 10.54 | A |
| ATOM | 4455 | O | ARG | A | 565 | 54.441 | 63.104 | −29.327 | 1.00 | 11.41 | A |
| ATOM | 4456 | N | GLU | A | 566 | 52.443 | 62.865 | −30.352 | 1.00 | 11.57 | A |
| ATOM | 4457 | CA | GLU | A | 566 | 52.500 | 64.139 | −31.048 | 1.00 | 12.67 | A |
| ATOM | 4458 | CB | GLU | A | 566 | 52.281 | 63.976 | −32.551 | 1.00 | 14.55 | A |
| ATOM | 4459 | CG | GLU | A | 566 | 53.360 | 63.207 | −33.265 | 1.00 | 16.81 | A |
| ATOM | 4460 | CD | GLU | A | 566 | 53.167 | 63.239 | −34.765 | 1.00 | 18.39 | A |
| ATOM | 4461 | OE1 | GLU | A | 566 | 52.025 | 63.017 | −35.219 | 1.00 | 20.87 | A |
| ATOM | 4462 | OE2 | GLU | A | 566 | 54.155 | 63.483 | −35.487 | 1.00 | 20.56 | A |
| ATOM | 4463 | C | GLU | A | 566 | 51.352 | 64.949 | −30.469 | 1.00 | 12.51 | A |
| ATOM | 4464 | O | GLU | A | 566 | 50.280 | 64.414 | −30.183 | 1.00 | 13.17 | A |
| ATOM | 4465 | N | GLN | A | 567 | 51.584 | 66.238 | −30.280 | 1.00 | 11.15 | A |
| ATOM | 4466 | CA | GLN | A | 567 | 50.559 | 67.117 | −29.744 | 1.00 | 11.24 | A |
| ATOM | 4467 | CB | GLN | A | 567 | 50.489 | 67.000 | −28.217 | 1.00 | 12.08 | A |
| ATOM | 4468 | CG | GLN | A | 567 | 49.477 | 67.948 | −27.579 | 1.00 | 11.81 | A |
| ATOM | 4469 | CD | GLN | A | 567 | 49.819 | 68.294 | −26.141 | 1.00 | 12.83 | A |
| ATOM | 4470 | OE1 | GLN | A | 567 | 49.777 | 67.441 | −25.255 | 1.00 | 11.48 | A |
| ATOM | 4471 | NE2 | GLN | A | 567 | 50.171 | 69.553 | −25.905 | 1.00 | 14.01 | A |
| ATOM | 4472 | C | GLN | A | 567 | 50.941 | 68.533 | −30.116 | 1.00 | 10.61 | A |
| ATOM | 4473 | O | GLN | A | 567 | 52.122 | 68.879 | −30.126 | 1.00 | 10.26 | A |
| ATOM | 4474 | N | LEU | A | 568 | 49.951 | 69.349 | −30.451 | 1.00 | 10.33 | A |
| ATOM | 4475 | CA | LEU | A | 568 | 50.247 | 70.734 | −30.766 | 1.00 | 9.94 | A |
| ATOM | 4476 | CB | LEU | A | 568 | 49.051 | 71.439 | −31.410 | 1.00 | 11.28 | A |
| ATOM | 4477 | CG | LEU | A | 568 | 48.657 | 71.080 | −32.842 | 1.00 | 11.61 | A |
| ATOM | 4478 | CD1 | LEU | A | 568 | 47.653 | 72.113 | −33.339 | 1.00 | 12.59 | A |
| ATOM | 4479 | CD2 | LEU | A | 568 | 49.884 | 71.073 | −33.745 | 1.00 | 12.50 | A |
| ATOM | 4480 | C | LEU | A | 568 | 50.568 | 71.426 | −29.452 | 1.00 | 10.03 | A |
| ATOM | 4481 | O | LEU | A | 568 | 49.926 | 71.171 | −28.430 | 1.00 | 10.24 | A |
| ATOM | 4482 | N | VAL | A | 569 | 51.581 | 72.279 | −29.477 | 1.00 | 8.96 | A |
| ATOM | 4483 | CA | VAL | A | 569 | 51.965 | 73.042 | −28.300 | 1.00 | 9.33 | A |
| ATOM | 4484 | CB | VAL | A | 569 | 53.331 | 72.585 | −27.724 | 1.00 | 8.58 | A |
| ATOM | 4485 | CG1 | VAL | A | 569 | 53.201 | 71.179 | −27.135 | 1.00 | 9.88 | A |
| ATOM | 4486 | CG2 | VAL | A | 569 | 54.402 | 72.607 | −28.808 | 1.00 | 9.77 | A |
| ATOM | 4487 | C | VAL | A | 569 | 52.046 | 74.503 | −28.716 | 1.00 | 9.59 | A |
| ATOM | 4488 | O | VAL | A | 569 | 52.314 | 74.815 | −29.877 | 1.00 | 10.28 | A |
| ATOM | 4489 | N | ASP | A | 570 | 51.790 | 75.405 | −27.779 | 1.00 | 9.31 | A |
| ATOM | 4490 | CA | ASP | A | 570 | 51.846 | 76.820 | −28.108 | 1.00 | 10.77 | A |
| ATOM | 4491 | CB | ASP | A | 570 | 50.434 | 77.409 | −28.195 | 1.00 | 12.92 | A |
| ATOM | 4492 | CG | ASP | A | 570 | 49.759 | 77.514 | −26.841 | 1.00 | 16.24 | A |
| ATOM | 4493 | OD1 | ASP | A | 570 | 49.620 | 76.479 | −26.159 | 1.00 | 20.02 | A |
| ATOM | 4494 | OD2 | ASP | A | 570 | 49.367 | 78.635 | −26.459 | 1.00 | 20.35 | A |
| ATOM | 4495 | C | ASP | A | 570 | 52.662 | 77.594 | −27.093 | 1.00 | 10.43 | A |
| ATOM | 4496 | O | ASP | A | 570 | 52.761 | 77.208 | −25.928 | 1.00 | 11.33 | A |
| ATOM | 4497 | N | PHE | A | 571 | 53.267 | 78.681 | −27.558 | 1.00 | 10.09 | A |
| ATOM | 4498 | CA | PHE | A | 571 | 54.069 | 79.546 | −26.704 | 1.00 | 10.04 | A |
| ATOM | 4499 | CB | PHE | A | 571 | 55.569 | 79.321 | −26.929 | 1.00 | 10.14 | A |
| ATOM | 4500 | CG | PHE | A | 571 | 56.064 | 77.980 | −26.482 | 1.00 | 9.15 | A |
| ATOM | 4501 | CD1 | PHE | A | 571 | 56.053 | 76.891 | −27.344 | 1.00 | 8.69 | A |
| ATOM | 4502 | CD2 | PHE | A | 571 | 56.544 | 77.809 | −25.190 | 1.00 | 9.56 | A |
| ATOM | 4503 | CE1 | PHE | A | 571 | 56.517 | 75.645 | −26.921 | 1.00 | 9.22 | A |
| ATOM | 4504 | CE2 | PHE | A | 571 | 57.006 | 76.570 | −24.758 | 1.00 | 8.84 | A |
| ATOM | 4505 | CZ | PHE | A | 571 | 56.991 | 75.489 | −25.628 | 1.00 | 9.29 | A |
| ATOM | 4506 | C | PHE | A | 571 | 53.761 | 80.984 | −27.075 | 1.00 | 10.51 | A |
| ATOM | 4507 | O | PHE | A | 571 | 53.379 | 81.263 | −28.212 | 1.00 | 11.02 | A |
| ATOM | 4508 | N | TYR | A | 572 | 53.918 | 81.890 | −26.114 | 1.00 | 10.59 | A |
| ATOM | 4509 | CA | TYR | A | 572 | 53.721 | 83.308 | −26.381 | 1.00 | 11.05 | A |
| ATOM | 4510 | CB | TYR | A | 572 | 53.298 | 84.069 | −25.123 | 1.00 | 12.13 | A |
| ATOM | 4511 | CG | TYR | A | 572 | 51.862 | 83.889 | −24.686 | 1.00 | 14.07 | A |
| ATOM | 4512 | CD1 | TYR | A | 572 | 50.897 | 83.376 | −25.550 | 1.00 | 14.81 | A |
| ATOM | 4513 | CE1 | TYR | A | 572 | 49.562 | 83.266 | −25.154 | 1.00 | 15.92 | A |
| ATOM | 4514 | CD2 | TYR | A | 572 | 51.461 | 84.286 | −23.412 | 1.00 | 16.47 | A |
| ATOM | 4515 | CE2 | TYR | A | 572 | 50.135 | 84.184 | −23.009 | 1.00 | 16.38 | A |
| ATOM | 4516 | CZ | TYR | A | 572 | 49.191 | 83.676 | −23.882 | 1.00 | 16.81 | A |
| ATOM | 4517 | OH | TYR | A | 572 | 47.874 | 83.592 | −23.481 | 1.00 | 17.51 | A |
| ATOM | 4518 | C | TYR | A | 572 | 55.078 | 83.839 | −26.826 | 1.00 | 11.07 | A |
| ATOM | 4519 | O | TYR | A | 572 | 56.094 | 83.557 | −26.188 | 1.00 | 10.80 | A |
| ATOM | 4520 | N | VAL | A | 573 | 55.096 | 84.596 | −27.920 | 1.00 | 10.87 | A |
| ATOM | 4521 | CA | VAL | A | 573 | 56.335 | 85.174 | −28.438 | 1.00 | 11.44 | A |
| ATOM | 4522 | CB | VAL | A | 573 | 56.782 | 84.475 | −29.745 | 1.00 | 11.72 | A |
| ATOM | 4523 | CG1 | VAL | A | 573 | 57.329 | 83.084 | −29.429 | 1.00 | 11.99 | A |
| ATOM | 4524 | CG2 | VAL | A | 573 | 55.613 | 84.371 | −30.710 | 1.00 | 12.32 | A |
| ATOM | 4525 | C | VAL | A | 573 | 56.135 | 86.667 | −28.699 | 1.00 | 11.37 | A |
| ATOM | 4526 | O | VAL | A | 573 | 55.021 | 87.107 | −28.967 | 1.00 | 12.01 | A |
| ATOM | 4527 | N | SER | A | 574 | 57.218 | 87.435 | −28.624 | 1.00 | 12.19 | A |
| ATOM | 4528 | CA | SER | A | 574 | 57.156 | 88.885 | −28.817 | 1.00 | 13.05 | A |
| ATOM | 4529 | CB | SER | A | 574 | 58.338 | 89.554 | −28.117 | 1.00 | 13.31 | A |
| ATOM | 4530 | OG | SER | A | 574 | 59.566 | 89.132 | −28.680 | 1.00 | 13.02 | A |
| ATOM | 4531 | C | SER | A | 574 | 57.116 | 89.347 | −30.270 | 1.00 | 13.99 | A |
| ATOM | 4532 | O | SER | A | 574 | 57.090 | 90.550 | −30.538 | 1.00 | 15.54 | A |
| ATOM | 4533 | N | SER | A | 575 | 57.120 | 88.402 | −31.203 | 1.00 | 14.20 | A |
| ATOM | 4534 | CA | SER | A | 575 | 57.071 | 88.732 | −32.621 | 1.00 | 14.88 | A |
| ATOM | 4535 | CB | SER | A | 575 | 58.483 | 88.846 | −33.197 | 1.00 | 15.07 | A |
| ATOM | 4536 | OG | SER | A | 575 | 58.440 | 88.996 | −34.607 | 1.00 | 16.15 | A |
| ATOM | 4537 | C | SER | A | 575 | 56.312 | 87.661 | −33.387 | 1.00 | 14.98 | A |
| ATOM | 4538 | O | SER | A | 575 | 56.349 | 86.488 | −33.027 | 1.00 | 14.24 | A |
| ATOM | 4539 | N | PRO | A | 576 | 55.599 | 88.055 | −34.450 | 1.00 | 15.06 | A |
| ATOM | 4540 | CD | PRO | A | 576 | 55.309 | 89.435 | −34.884 | 1.00 | 15.77 | A |
| ATOM | 4541 | CA | PRO | A | 576 | 54.845 | 87.084 | −35.244 | 1.00 | 14.91 | A |
| ATOM | 4542 | CB | PRO | A | 576 | 53.797 | 87.950 | −35.929 | 1.00 | 15.59 | A |
| ATOM | 4543 | CG | PRO | A | 576 | 54.558 | 89.221 | −36.184 | 1.00 | 16.60 | A |
| ATOM | 4544 | C | PRO | A | 576 | 55.763 | 86.385 | −36.246 | 1.00 | 14.44 | A |
| ATOM | 4545 | O | PRO | A | 576 | 55.411 | 85.360 | −36.821 | 1.00 | 14.89 | A |
| ATOM | 4546 | N | PHE | A | 577 | 56.951 | 86.948 | −36.443 | 1.00 | 14.84 | A |
| ATOM | 4547 | CA | PHE | A | 577 | 57.912 | 86.400 | −37.390 | 1.00 | 15.08 | A |
| ATOM | 4548 | CB | PHE | A | 577 | 58.680 | 87.546 | −38.050 | 1.00 | 16.28 | A |
| ATOM | 4549 | CG | PHE | A | 577 | 57.790 | 88.549 | −38.732 | 1.00 | 18.45 | A |
| ATOM | 4550 | CD1 | PHE | A | 577 | 58.042 | 89.913 | −38.617 | 1.00 | 19.42 | A |
| ATOM | 4551 | CD2 | PHE | A | 577 | 56.699 | 88.131 | −39.487 | 1.00 | 18.51 | A |
| ATOM | 4552 | CE1 | PHE | A | 577 | 57.218 | 90.847 | −39.243 | 1.00 | 20.70 | A |
| ATOM | 4553 | CE2 | PHE | A | 577 | 55.867 | 89.059 | −40.119 | 1.00 | 20.04 | A |
| ATOM | 4554 | CZ | PHE | A | 577 | 56.129 | 90.418 | −39.995 | 1.00 | 20.44 | A |
| ATOM | 4555 | C | PHE | A | 577 | 58.870 | 85.440 | −36.703 | 1.00 | 14.78 | A |
| ATOM | 4556 | O | PHE | A | 577 | 60.053 | 85.728 | −36.528 | 1.00 | 14.56 | A |
| ATOM | 4557 | N | VAL | A | 578 | 58.335 | 84.288 | −36.317 | 1.00 | 14.00 | A |
| ATOM | 4558 | CA | VAL | A | 578 | 59.112 | 83.268 | −35.635 | 1.00 | 14.21 | A |
| ATOM | 4559 | CB | VAL | A | 578 | 58.641 | 83.116 | −34.172 | 1.00 | 14.17 | A |
| ATOM | 4560 | CG1 | VAL | A | 578 | 59.412 | 82.000 | −33.481 | 1.00 | 14.20 | A |
| ATOM | 4561 | CG2 | VAL | A | 578 | 58.840 | 84.429 | −33.431 | 1.00 | 14.11 | A |
| ATOM | 4562 | C | VAL | A | 578 | 58.971 | 81.932 | −36.352 | 1.00 | 13.94 | A |
| ATOM | 4563 | O | VAL | A | 578 | 57.887 | 81.567 | −36.806 | 1.00 | 14.31 | A |
| ATOM | 4564 | N | SER | A | 579 | 60.083 | 81.218 | −36.465 | 1.00 | 14.83 | A |
| ATOM | 4565 | CA | SER | A | 579 | 60.096 | 79.920 | −37.117 | 1.00 | 14.80 | A |
| ATOM | 4566 | CB | SER | A | 579 | 60.997 | 79.956 | −38.356 | 1.00 | 16.06 | A |
| ATOM | 4567 | OG | SER | A | 579 | 62.294 | 80.415 | −38.032 | 1.00 | 19.90 | A |
| ATOM | 4568 | C | SER | A | 579 | 60.588 | 78.875 | −36.127 | 1.00 | 14.00 | A |
| ATOM | 4569 | O | SER | A | 579 | 61.340 | 79.188 | −35.204 | 1.00 | 13.81 | A |
| ATOM | 4570 | N | VAL | A | 580 | 60.157 | 77.637 | −36.326 | 1.00 | 12.34 | A |
| ATOM | 4571 | CA | VAL | A | 580 | 60.528 | 76.542 | −35.444 | 1.00 | 12.09 | A |
| ATOM | 4572 | CB | VAL | A | 580 | 59.269 | 75.835 | −34.898 | 1.00 | 11.61 | A |
| ATOM | 4573 | CG1 | VAL | A | 580 | 59.662 | 74.778 | −33.871 | 1.00 | 11.03 | A |
| ATOM | 4574 | CG2 | VAL | A | 580 | 58.321 | 76.858 | −34.284 | 1.00 | 10.91 | A |
| ATOM | 4575 | C | VAL | A | 580 | 61.388 | 75.498 | −36.144 | 1.00 | 12.49 | A |
| ATOM | 4576 | O | VAL | A | 580 | 61.185 | 75.192 | −37.320 | 1.00 | 13.11 | A |
| ATOM | 4577 | N | THR | A | 581 | 62.347 | 74.956 | −35.402 | 1.00 | 12.19 | A |
| ATOM | 4578 | CA | THR | A | 581 | 63.236 | 73.911 | −35.894 | 1.00 | 13.34 | A |
| ATOM | 4579 | CB | THR | A | 581 | 64.618 | 74.470 | −36.315 | 1.00 | 13.96 | A |
| ATOM | 4580 | OG1 | THR | A | 581 | 65.119 | 75.344 | −35.295 | 1.00 | 15.16 | A |
| ATOM | 4581 | CG2 | THR | A | 581 | 64.516 | 75.220 | −37.632 | 1.00 | 13.79 | A |
| ATOM | 4582 | C | THR | A | 581 | 63.465 | 72.918 | −34.762 | 1.00 | 14.12 | A |
| ATOM | 4583 | O | THR | A | 581 | 63.316 | 73.269 | −33.593 | 1.00 | 13.78 | A |
| ATOM | 4584 | N | ASP | A | 582 | 63.791 | 71.676 | −35.104 | 1.00 | 15.79 | A |
| ATOM | 4585 | CA | ASP | A | 582 | 64.086 | 70.687 | −34.075 | 1.00 | 17.41 | A |
| ATOM | 4586 | CB | ASP | A | 582 | 63.674 | 69.271 | −34.522 | 1.00 | 17.61 | A |
| ATOM | 4587 | CG | ASP | A | 582 | 64.382 | 68.801 | −35.782 | 1.00 | 17.96 | A |
| ATOM | 4588 | OD1 | ASP | A | 582 | 63.942 | 67.774 | −36.345 | 1.00 | 19.58 | A |
| ATOM | 4589 | OD2 | ASP | A | 582 | 65.367 | 69.434 | −36.205 | 1.00 | 17.28 | A |
| ATOM | 4590 | C | ASP | A | 582 | 65.592 | 70.831 | −33.866 | 1.00 | 19.39 | A |
| ATOM | 4591 | O | ASP | A | 582 | 66.209 | 71.675 | −34.512 | 1.00 | 18.93 | A |
| ATOM | 4592 | N | LEU | A | 583 | 66.205 | 70.052 | −32.983 | 1.00 | 21.85 | A |
| ATOM | 4593 | CA | LEU | A | 583 | 67.636 | 70.251 | −32.777 | 1.00 | 23.92 | A |
| ATOM | 4594 | CB | LEU | A | 583 | 68.138 | 69.489 | −31.549 | 1.00 | 25.99 | A |
| ATOM | 4595 | CG | LEU | A | 583 | 69.423 | 70.140 | −31.024 | 1.00 | 27.28 | A |
| ATOM | 4596 | CD1 | LEU | A | 583 | 69.104 | 71.550 | −30.548 | 1.00 | 28.01 | A |
| ATOM | 4597 | CD2 | LEU | A | 583 | 70.019 | 69.329 | −29.898 | 1.00 | 28.61 | A |
| ATOM | 4598 | C | LEU | A | 583 | 68.491 | 69.897 | −33.988 | 1.00 | 24.12 | A |
| ATOM | 4599 | O | LEU | A | 583 | 69.615 | 70.385 | −34.120 | 1.00 | 25.14 | A |
| ATOM | 4600 | N | ALA | A | 584 | 67.965 | 69.055 | −34.873 | 1.00 | 23.00 | A |
| ATOM | 4601 | CA | ALA | A | 584 | 68.703 | 68.680 | −36.074 | 1.00 | 21.20 | A |
| ATOM | 4602 | CB | ALA | A | 584 | 68.157 | 67.380 | −36.649 | 1.00 | 21.31 | A |
| ATOM | 4603 | C | ALA | A | 584 | 68.561 | 69.808 | −37.091 | 1.00 | 20.45 | A |
| ATOM | 4604 | O | ALA | A | 584 | 68.994 | 69.693 | −38.237 | 1.00 | 19.17 | A |
| ATOM | 4605 | N | ASN | A | 585 | 67.944 | 70.901 | −36.651 | 1.00 | 20.39 | A |
| ATOM | 4606 | CA | ASN | A | 585 | 67.725 | 72.075 | −37.487 | 1.00 | 20.49 | A |
| ATOM | 4607 | CB | ASN | A | 585 | 69.052 | 72.580 | −38.058 | 1.00 | 22.80 | A |
| ATOM | 4608 | CG | ASN | A | 585 | 69.389 | 73.979 | −37.585 | 1.00 | 24.67 | A |
| ATOM | 4609 | OD1 | ASN | A | 585 | 68.643 | 74.928 | −37.833 | 1.00 | 25.57 | A |
| ATOM | 4610 | ND2 | ASN | A | 585 | 70.514 | 74.114 | −36.891 | 1.00 | 26.67 | A |
| ATOM | 4611 | C | ASN | A | 585 | 66.727 | 71.848 | −38.616 | 1.00 | 19.93 | A |
| ATOM | 4612 | O | ASN | A | 585 | 66.715 | 72.592 | −39.603 | 1.00 | 20.20 | A |
| ATOM | 4613 | N | ASN | A | 586 | 65.894 | 70.822 | −38.473 | 1.00 | 18.43 | A |
| ATOM | 4614 | CA | ASN | A | 586 | 64.872 | 70.525 | −39.471 | 1.00 | 17.78 | A |
| ATOM | 4615 | CB | ASN | A | 586 | 64.329 | 69.101 | −39.317 | 1.00 | 18.38 | A |
| ATOM | 4616 | CG | ASN | A | 586 | 65.396 | 68.045 | −39.465 | 1.00 | 18.83 | A |
| ATOM | 4617 | OD1 | ASN | A | 586 | 66.205 | 68.089 | −40.388 | 1.00 | 18.59 | A |
| ATOM | 4618 | ND2 | ASN | A | 586 | 65.392 | 67.073 | −38.560 | 1.00 | 18.31 | A |
| ATOM | 4619 | C | ASN | A | 586 | 63.720 | 71.492 | −39.241 | 1.00 | 17.17 | A |
| ATOM | 4620 | O | ASN | A | 586 | 63.244 | 71.640 | −38.118 | 1.00 | 15.87 | A |
| ATOM | 4621 | N | PRO | A | 587 | 63.261 | 72.173 | −40.296 | 1.00 | 16.47 | A |
| ATOM | 4622 | CD | PRO | A | 587 | 63.673 | 72.155 | −41.710 | 1.00 | 17.25 | A |
| ATOM | 4623 | CA | PRO | A | 587 | 62.149 | 73.099 | −40.080 | 1.00 | 15.85 | A |
| ATOM | 4624 | CB | PRO | A | 587 | 61.983 | 73.762 | −41.447 | 1.00 | 16.75 | A |
| ATOM | 4625 | CG | PRO | A | 587 | 62.443 | 72.697 | −42.400 | 1.00 | 17.77 | A |
| ATOM | 4626 | C | PRO | A | 587 | 60.894 | 72.354 | −39.630 | 1.00 | 14.91 | A |
| ATOM | 4627 | O | PRO | A | 587 | 60.666 | 71.203 | −40.009 | 1.00 | 15.09 | A |
| ATOM | 4628 | N | VAL | A | 588 | 60.096 | 73.008 | −38.795 | 1.00 | 13.83 | A |
| ATOM | 4629 | CA | VAL | A | 588 | 58.857 | 72.428 | −38.298 | 1.00 | 13.91 | A |
| ATOM | 4630 | CB | VAL | A | 588 | 58.902 | 72.242 | −36.764 | 1.00 | 14.00 | A |
| ATOM | 4631 | CG1 | VAL | A | 588 | 57.570 | 71.711 | −36.263 | 1.00 | 14.23 | A |
| ATOM | 4632 | CG2 | VAL | A | 588 | 60.028 | 71.285 | −36.390 | 1.00 | 13.60 | A |
| ATOM | 4633 | C | VAL | A | 588 | 57.733 | 73.395 | −38.655 | 1.00 | 13.82 | A |
| ATOM | 4634 | O | VAL | A | 588 | 57.829 | 74.590 | −38.371 | 1.00 | 13.34 | A |
| ATOM | 4635 | N | GLU | A | 589 | 56.677 | 72.886 | −39.282 | 1.00 | 14.01 | A |
| ATOM | 4636 | CA | GLU | A | 589 | 55.559 | 73.740 | −39.666 | 1.00 | 14.78 | A |
| ATOM | 4637 | CB | GLU | A | 589 | 54.492 | 72.945 | −40.417 | 1.00 | 16.78 | A |
| ATOM | 4638 | CG | GLU | A | 589 | 53.508 | 73.841 | −41.153 | 1.00 | 20.93 | A |
| ATOM | 4639 | CD | GLU | A | 589 | 52.338 | 73.083 | −41.743 | 1.00 | 22.74 | A |
| ATOM | 4640 | OE1 | GLU | A | 589 | 52.543 | 71.952 | −42.229 | 1.00 | 25.02 | A |
| ATOM | 4641 | OE2 | GLU | A | 589 | 51.213 | 73.628 | −41.733 | 1.00 | 24.68 | A |
| ATOM | 4642 | C | GLU | A | 589 | 54.940 | 74.370 | −38.428 | 1.00 | 14.21 | A |
| ATOM | 4643 | O | GLU | A | 589 | 54.679 | 73.688 | −37.436 | 1.00 | 13.85 | A |
| ATOM | 4644 | N | ALA | A | 590 | 54.703 | 75.673 | −38.491 | 1.00 | 13.29 | A |
| ATOM | 4645 | CA | ALA | A | 590 | 54.130 | 76.387 | −37.365 | 1.00 | 12.68 | A |
| ATOM | 4646 | CB | ALA | A | 590 | 55.215 | 77.169 | −36.643 | 1.00 | 12.81 | A |
| ATOM | 4647 | C | ALA | A | 590 | 53.026 | 77.329 | −37.804 | 1.00 | 12.30 | A |
| ATOM | 4648 | O | ALA | A | 590 | 52.925 | 77.691 | −38.979 | 1.00 | 12.10 | A |
| ATOM | 4649 | N | GLN | A | 591 | 52.196 | 77.714 | −36.842 | 1.00 | 11.58 | A |
| ATOM | 4650 | CA | GLN | A | 591 | 51.099 | 78.640 | −37.079 | 1.00 | 10.88 | A |
| ATOM | 4651 | CB | GLN | A | 591 | 49.746 | 77.924 | −37.004 | 1.00 | 11.34 | A |
| ATOM | 4652 | CG | GLN | A | 591 | 48.551 | 78.880 | −37.069 | 1.00 | 11.63 | A |
| ATOM | 4653 | CD | GLN | A | 591 | 47.220 | 78.186 | −36.836 | 1.00 | 11.31 | A |
| ATOM | 4654 | OE1 | GLN | A | 591 | 46.948 | 77.136 | −37.416 | 1.00 | 12.28 | A |
| ATOM | 4655 | NE2 | GLN | A | 591 | 46.380 | 78.777 | −35.993 | 1.00 | 11.76 | A |
| ATOM | 4656 | C | GLN | A | 591 | 51.140 | 79.714 | −36.007 | 1.00 | 11.07 | A |
| ATOM | 4657 | O | GLN | A | 591 | 51.335 | 79.419 | −34.825 | 1.00 | 11.93 | A |
| ATOM | 4658 | N | VAL | A | 592 | 50.978 | 80.966 | −36.416 | 1.00 | 10.29 | A |
| ATOM | 4659 | CA | VAL | A | 592 | 50.960 | 82.059 | −35.461 | 1.00 | 11.22 | A |
| ATOM | 4660 | CB | VAL | A | 592 | 51.983 | 83.158 | −35.831 | 1.00 | 11.59 | A |
| ATOM | 4661 | CG1 | VAL | A | 592 | 51.742 | 84.405 | −34.990 | 1.00 | 12.13 | A |
| ATOM | 4662 | CG2 | VAL | A | 592 | 53.399 | 82.641 | −35.584 | 1.00 | 11.16 | A |
| ATOM | 4663 | C | VAL | A | 592 | 49.550 | 82.637 | −35.433 | 1.00 | 11.38 | A |
| ATOM | 4664 | O | VAL | A | 592 | 48.935 | 82.853 | −36.477 | 1.00 | 12.01 | A |
| ATOM | 4665 | N | SER | A | 593 | 49.038 | 82.848 | −34.227 | 1.00 | 12.29 | A |
| ATOM | 4666 | CA | SER | A | 593 | 47.704 | 83.399 | −34.020 | 1.00 | 12.41 | A |
| ATOM | 4667 | CB | SER | A | 593 | 46.757 | 82.337 | −33.444 | 1.00 | 12.53 | A |
| ATOM | 4668 | OG | SER | A | 593 | 46.554 | 81.255 | −34.339 | 1.00 | 13.81 | A |
| ATOM | 4669 | C | SER | A | 593 | 47.833 | 84.533 | −33.018 | 1.00 | 12.72 | A |
| ATOM | 4670 | O | SER | A | 593 | 48.828 | 84.629 | −32.297 | 1.00 | 13.46 | A |
| ATOM | 4671 | N | PRO | A | 594 | 46.831 | 85.415 | −32.959 | 1.00 | 12.94 | A |
| ATOM | 4672 | CD | PRO | A | 594 | 45.693 | 85.588 | −33.877 | 1.00 | 12.56 | A |
| ATOM | 4673 | CA | PRO | A | 594 | 46.909 | 86.523 | −32.004 | 1.00 | 12.57 | A |
| ATOM | 4674 | CB | PRO | A | 594 | 45.802 | 87.471 | −32.471 | 1.00 | 13.02 | A |
| ATOM | 4675 | CG | PRO | A | 594 | 45.560 | 87.083 | −33.915 | 1.00 | 12.90 | A |
| ATOM | 4676 | C | PRO | A | 594 | 46.641 | 86.038 | −30.583 | 1.00 | 12.82 | A |
| ATOM | 4677 | O | PRO | A | 594 | 46.185 | 84.913 | −30.374 | 1.00 | 12.85 | A |
| ATOM | 4678 | N | VAL | A | 595 | 46.943 | 86.882 | −29.605 | 1.00 | 11.95 | A |
| ATOM | 4679 | CA | VAL | A | 595 | 46.650 | 86.549 | −28.221 | 1.00 | 12.90 | A |
| ATOM | 4680 | CB | VAL | A | 595 | 47.761 | 87.006 | −27.249 | 1.00 | 12.80 | A |
| ATOM | 4681 | CG1 | VAL | A | 595 | 47.280 | 86.842 | −25.811 | 1.00 | 13.35 | A |
| ATOM | 4682 | CG2 | VAL | A | 595 | 49.020 | 86.176 | −27.467 | 1.00 | 13.76 | A |
| ATOM | 4683 | C | VAL | A | 595 | 45.366 | 87.315 | −27.927 | 1.00 | 13.06 | A |
| ATOM | 4684 | O | VAL | A | 595 | 45.369 | 88.545 | −27.845 | 1.00 | 14.57 | A |
| ATOM | 4685 | N | TRP | A | 596 | 44.264 | 86.583 | −27.805 | 1.00 | 13.30 | A |
| ATOM | 4686 | CA | TRP | A | 596 | 42.967 | 87.187 | −27.539 | 1.00 | 14.05 | A |
| ATOM | 4687 | CB | TRP | A | 596 | 41.879 | 86.522 | −28.391 | 1.00 | 13.01 | A |
| ATOM | 4688 | CG | TRP | A | 596 | 42.037 | 86.706 | −29.862 | 1.00 | 13.11 | A |
| ATOM | 4689 | CD2 | TRP | A | 596 | 41.728 | 87.883 | −30.615 | 1.00 | 13.30 | A |
| ATOM | 4690 | CE2 | TRP | A | 596 | 42.009 | 87.600 | −31.969 | 1.00 | 13.52 | A |
| ATOM | 4691 | CE3 | TRP | A | 596 | 41.241 | 89.153 | −30.275 | 1.00 | 13.42 | A |
| ATOM | 4692 | CD1 | TRP | A | 596 | 42.483 | 85.780 | −30.765 | 1.00 | 13.06 | A |
| ATOM | 4693 | NE1 | TRP | A | 596 | 42.467 | 86.310 | −32.034 | 1.00 | 13.17 | A |
| ATOM | 4694 | CZ2 | TRP | A | 596 | 41.818 | 88.541 | −32.987 | 1.00 | 13.38 | A |
| ATOM | 4695 | CZ3 | TRP | A | 596 | 41.050 | 90.089 | −31.288 | 1.00 | 13.71 | A |
| ATOM | 4696 | CH2 | TRP | A | 596 | 41.339 | 89.776 | −32.627 | 1.00 | 14.26 | A |
| ATOM | 4697 | C | TRP | A | 596 | 42.567 | 87.072 | −26.078 | 1.00 | 15.30 | A |
| ATOM | 4698 | O | TRP | A | 596 | 42.741 | 86.024 | −25.456 | 1.00 | 15.31 | A |
| ATOM | 4699 | N | SER | A | 597 | 42.027 | 88.159 | −25.540 | 1.00 | 16.68 | A |
| ATOM | 4700 | CA | SER | A | 597 | 41.559 | 88.187 | −24.164 | 1.00 | 18.42 | A |
| ATOM | 4701 | CB | SER | A | 597 | 42.486 | 89.040 | −23.298 | 1.00 | 19.42 | A |
| ATOM | 4702 | OG | SER | A | 597 | 42.563 | 90.359 | −23.797 | 1.00 | 21.78 | A |
| ATOM | 4703 | C | SER | A | 597 | 40.160 | 88.788 | −24.185 | 1.00 | 18.74 | A |
| ATOM | 4704 | O | SER | A | 597 | 39.907 | 89.768 | −24.892 | 1.00 | 19.83 | A |
| ATOM | 4705 | N | TRP | A | 598 | 39.249 | 88.194 | −23.425 | 1.00 | 18.70 | A |
| ATOM | 4706 | CA | TRP | A | 598 | 37.881 | 88.683 | −23.377 | 1.00 | 19.40 | A |
| ATOM | 4707 | CB | TRP | A | 598 | 36.896 | 87.515 | −23.328 | 1.00 | 16.72 | A |
| ATOM | 4708 | CG | TRP | A | 598 | 36.870 | 86.734 | −24.603 | 1.00 | 14.05 | A |
| ATOM | 4709 | CD2 | TRP | A | 598 | 35.866 | 86.791 | −25.621 | 1.00 | 13.09 | A |
| ATOM | 4710 | CE2 | TRP | A | 598 | 36.272 | 85.922 | −26.656 | 1.00 | 12.49 | A |
| ATOM | 4711 | CE3 | TRP | A | 598 | 34.660 | 87.494 | −25.760 | 1.00 | 12.76 | A |
| ATOM | 4712 | CD1 | TRP | A | 598 | 37.817 | 85.859 | −25.048 | 1.00 | 13.40 | A |
| ATOM | 4713 | NE1 | TRP | A | 598 | 37.466 | 85.366 | −26.281 | 1.00 | 12.12 | A |
| ATOM | 4714 | CZ2 | TRP | A | 598 | 35.516 | 85.734 | −27.816 | 1.00 | 12.90 | A |
| ATOM | 4715 | CZ3 | TRP | A | 598 | 33.907 | 87.306 | −26.917 | 1.00 | 12.25 | A |
| ATOM | 4716 | CH2 | TRP | A | 598 | 34.341 | 86.433 | −27.929 | 1.00 | 12.83 | A |
| ATOM | 4717 | C | TRP | A | 598 | 37.673 | 89.595 | −22.183 | 1.00 | 21.86 | A |
| ATOM | 4718 | O | TRP | A | 598 | 38.185 | 89.340 | −21.093 | 1.00 | 22.28 | A |
| ATOM | 4719 | N | HIS | A | 599 | 36.917 | 90.663 | −22.397 | 1.00 | 25.14 | A |
| ATOM | 4720 | CA | HIS | A | 599 | 36.662 | 91.627 | −21.341 | 1.00 | 29.11 | A |
| ATOM | 4721 | CB | HIS | A | 599 | 37.513 | 92.875 | −21.575 | 1.00 | 30.89 | A |
| ATOM | 4722 | CG | HIS | A | 599 | 38.982 | 92.597 | −21.638 | 1.00 | 32.98 | A |
| ATOM | 4723 | CD2 | HIS | A | 599 | 39.844 | 92.597 | −22.682 | 1.00 | 33.68 | A |
| ATOM | 4724 | ND1 | HIS | A | 599 | 39.722 | 92.242 | −20.531 | 1.00 | 33.67 | A |
| ATOM | 4725 | CE1 | HIS | A | 599 | 40.977 | 92.036 | −20.890 | 1.00 | 34.01 | A |
| ATOM | 4726 | NE2 | HIS | A | 599 | 41.078 | 92.246 | −22.190 | 1.00 | 34.05 | A |
| ATOM | 4727 | C | HIS | A | 599 | 35.197 | 92.018 | −21.262 | 1.00 | 31.06 | A |
| ATOM | 4728 | O | HIS | A | 599 | 34.520 | 92.156 | −22.281 | 1.00 | 30.43 | A |
| ATOM | 4729 | N | HIS | A | 600 | 34.712 | 92.188 | −20.037 | 1.00 | 33.98 | A |
| ATOM | 4730 | CA | HIS | A | 600 | 33.333 | 92.589 | −19.818 | 1.00 | 37.06 | A |
| ATOM | 4731 | CB | HIS | A | 600 | 32.835 | 92.083 | −18.463 | 1.00 | 38.91 | A |
| ATOM | 4732 | CG | HIS | A | 600 | 31.366 | 92.279 | −18.250 | 1.00 | 40.97 | A |
| ATOM | 4733 | CD2 | HIS | A | 600 | 30.390 | 91.404 | −17.910 | 1.00 | 41.66 | A |
| ATOM | 4734 | ND1 | HIS | A | 600 | 30.750 | 93.504 | −18.389 | 1.00 | 41.78 | A |
| ATOM | 4735 | CE1 | HIS | A | 600 | 29.458 | 93.376 | −18.145 | 1.00 | 42.26 | A |
| ATOM | 4736 | NE2 | HIS | A | 600 | 29.213 | 92.111 | −17.852 | 1.00 | 42.52 | A |
| ATOM | 4737 | C | HIS | A | 600 | 33.339 | 94.109 | −19.837 | 1.00 | 37.98 | A |
| ATOM | 4738 | O | HIS | A | 600 | 33.560 | 94.752 | −18.811 | 1.00 | 38.39 | A |
| ATOM | 4739 | N | ASP | A | 601 | 33.113 | 94.677 | −21.016 | 1.00 | 39.04 | A |
| ATOM | 4740 | CA | ASP | A | 601 | 33.108 | 96.123 | −21.185 | 1.00 | 40.24 | A |
| ATOM | 4741 | CB | ASP | A | 601 | 32.909 | 96.472 | −22.660 | 1.00 | 41.10 | A |
| ATOM | 4742 | CG | ASP | A | 601 | 33.283 | 97.906 | −22.976 | 1.00 | 42.18 | A |
| ATOM | 4743 | OD1 | ASP | A | 601 | 32.722 | 98.825 | −22.344 | 1.00 | 42.37 | A |
| ATOM | 4744 | OD2 | ASP | A | 601 | 34.139 | 98.112 | −23.862 | 1.00 | 42.54 | A |
| ATOM | 4745 | C | ASP | A | 601 | 32.010 | 96.768 | −20.344 | 1.00 | 40.49 | A |
| ATOM | 4746 | O | ASP | A | 601 | 30.829 | 96.678 | −20.675 | 1.00 | 40.39 | A |
| ATOM | 4747 | N | THR | A | 602 | 32.413 | 97.415 | −19.254 | 1.00 | 40.63 | A |
| ATOM | 4748 | CA | THR | A | 602 | 31.476 | 98.077 | −18.352 | 1.00 | 40.68 | A |
| ATOM | 4749 | CB | THR | A | 602 | 32.200 | 98.623 | −17.102 | 1.00 | 41.47 | A |
| ATOM | 4750 | OG1 | THR | A | 602 | 32.869 | 97.550 | −16.429 | 1.00 | 42.23 | A |
| ATOM | 4751 | CG2 | THR | A | 602 | 31.204 | 99.271 | −16.146 | 1.00 | 42.22 | A |
| ATOM | 4752 | C | THR | A | 602 | 30.762 | 99.237 | −19.039 | 1.00 | 39.69 | A |
| ATOM | 4753 | O | THR | A | 602 | 29.652 | 99.608 | −18.656 | 1.00 | 40.04 | A |
| ATOM | 4754 | N | LEU | A | 603 | 31.404 | 99.804 | −20.056 | 1.00 | 38.12 | A |
| ATOM | 4755 | CA | LEU | A | 603 | 30.834 | 100.927 | −20.790 | 1.00 | 36.37 | A |
| ATOM | 4756 | CB | LEU | A | 603 | 31.944 | 101.732 | −21.478 | 1.00 | 37.67 | A |
| ATOM | 4757 | CG | LEU | A | 603 | 32.888 | 102.554 | −20.593 | 1.00 | 38.43 | A |
| ATOM | 4758 | CD1 | LEU | A | 603 | 32.080 | 103.570 | −19.799 | 1.00 | 38.88 | A |
| ATOM | 4759 | CD2 | LEU | A | 603 | 33.665 | 101.640 | −19.659 | 1.00 | 39.02 | A |
| ATOM | 4760 | C | LEU | A | 603 | 29.800 | 100.499 | −21.825 | 1.00 | 34.34 | A |
| ATOM | 4761 | O | LEU | A | 603 | 28.645 | 100.915 | −21.763 | 1.00 | 33.94 | A |
| ATOM | 4762 | N | THR | A | 604 | 30.217 | 99.667 | −22.775 | 1.00 | 31.91 | A |
| ATOM | 4763 | CA | THR | A | 604 | 29.321 | 99.199 | −23.828 | 1.00 | 29.56 | A |
| ATOM | 4764 | CB | THR | A | 604 | 30.110 | 98.674 | −25.040 | 1.00 | 29.91 | A |
| ATOM | 4765 | OG1 | THR | A | 604 | 30.920 | 97.561 | −24.639 | 1.00 | 30.03 | A |
| ATOM | 4766 | CG2 | THR | A | 604 | 31.004 | 99.768 | −25.608 | 1.00 | 29.97 | A |
| ATOM | 4767 | C | THR | A | 604 | 28.385 | 98.092 | −23.355 | 1.00 | 27.84 | A |
| ATOM | 4768 | O | THR | A | 604 | 27.447 | 97.722 | −24.060 | 1.00 | 27.19 | A |
| ATOM | 4769 | N | LYS | A | 605 | 28.642 | 97.567 | −22.162 | 1.00 | 26.35 | A |
| ATOM | 4770 | CA | LYS | A | 605 | 27.819 | 96.499 | −21.605 | 1.00 | 25.31 | A |
| ATOM | 4771 | CB | LYS | A | 605 | 26.381 | 96.985 | −21.411 | 1.00 | 25.79 | A |
| ATOM | 4772 | CG | LYS | A | 605 | 26.247 | 98.128 | −20.418 | 1.00 | 26.37 | A |
| ATOM | 4773 | CD | LYS | A | 605 | 26.700 | 97.703 | −19.028 | 1.00 | 26.73 | A |
| ATOM | 4774 | CE | LYS | A | 605 | 26.600 | 98.853 | −18.038 | 1.00 | 26.90 | A |
| ATOM | 4775 | NZ | LYS | A | 605 | 27.012 | 98.438 | −16.669 | 1.00 | 27.43 | A |
| ATOM | 4776 | C | LYS | A | 605 | 27.831 | 95.270 | −22.507 | 1.00 | 24.34 | A |
| ATOM | 4777 | O | LYS | A | 605 | 26.802 | 94.624 | −22.708 | 1.00 | 24.15 | A |
| ATOM | 4778 | N | THR | A | 606 | 29.000 | 94.959 | −23.055 | 1.00 | 22.88 | A |
| ATOM | 4779 | CA | THR | A | 606 | 29.157 | 93.801 | −23.925 | 1.00 | 21.84 | A |
| ATOM | 4780 | CB | THR | A | 606 | 29.277 | 94.207 | −25.412 | 1.00 | 22.73 | A |
| ATOM | 4781 | OG1 | THR | A | 606 | 30.419 | 95.057 | −25.584 | 1.00 | 23.70 | A |
| ATOM | 4782 | CG2 | THR | A | 606 | 28.024 | 94.932 | −25.873 | 1.00 | 23.17 | A |
| ATOM | 4783 | C | THR | A | 606 | 30.417 | 93.038 | −23.545 | 1.00 | 20.46 | A |
| ATOM | 4784 | O | THR | A | 606 | 31.310 | 93.575 | −22.893 | 1.00 | 20.36 | A |
| ATOM | 4785 | N | ILE | A | 607 | 30.473 | 91.775 | −23.948 | 1.00 | 19.22 | A |
| ATOM | 4786 | CA | ILE | A | 607 | 31.627 | 90.930 | −23.678 | 1.00 | 18.27 | A |
| ATOM | 4787 | CB | ILE | A | 607 | 31.194 | 89.579 | −23.081 | 1.00 | 18.14 | A |
| ATOM | 4788 | CG2 | ILE | A | 607 | 32.419 | 88.737 | −22.744 | 1.00 | 18.10 | A |
| ATOM | 4789 | CG1 | ILE | A | 607 | 30.362 | 89.819 | −21.818 | 1.00 | 18.82 | A |
| ATOM | 4790 | CD1 | ILE | A | 607 | 29.723 | 88.569 | −21.261 | 1.00 | 20.02 | A |
| ATOM | 4791 | C | ILE | A | 607 | 32.291 | 90.705 | −25.032 | 1.00 | 17.90 | A |
| ATOM | 4792 | O | ILE | A | 607 | 31.746 | 90.012 | −25.891 | 1.00 | 17.34 | A |
| ATOM | 4793 | N | HIS | A | 608 | 33.458 | 91.307 | −25.229 | 1.00 | 17.94 | A |
| ATOM | 4794 | CA | HIS | A | 608 | 34.155 | 91.173 | −26.498 | 1.00 | 18.20 | A |
| ATOM | 4795 | CB | HIS | A | 608 | 33.909 | 92.409 | −27.365 | 1.00 | 19.84 | A |
| ATOM | 4796 | CG | HIS | A | 608 | 34.468 | 93.673 | −26.792 | 1.00 | 21.38 | A |
| ATOM | 4797 | CD2 | HIS | A | 608 | 35.447 | 94.494 | −27.238 | 1.00 | 22.64 | A |
| ATOM | 4798 | ND1 | HIS | A | 608 | 34.012 | 94.225 | −25.614 | 1.00 | 22.82 | A |
| ATOM | 4799 | CE1 | HIS | A | 608 | 34.686 | 95.332 | −25.360 | 1.00 | 22.93 | A |
| ATOM | 4800 | NE2 | HIS | A | 608 | 35.563 | 95.518 | −26.330 | 1.00 | 23.21 | A |
| ATOM | 4801 | C | HIS | A | 608 | 35.649 | 90.949 | −26.317 | 1.00 | 17.54 | A |
| ATOM | 4802 | O | HIS | A | 608 | 36.212 | 91.246 | −25.262 | 1.00 | 17.84 | A |
| ATOM | 4803 | N | PRO | A | 609 | 36.313 | 90.423 | −27.355 | 1.00 | 16.61 | A |
| ATOM | 4804 | CD | PRO | A | 609 | 35.716 | 89.852 | −28.578 | 1.00 | 15.98 | A |
| ATOM | 4805 | CA | PRO | A | 609 | 37.748 | 90.148 | −27.310 | 1.00 | 16.72 | A |
| ATOM | 4806 | CB | PRO | A | 609 | 37.873 | 88.927 | −28.203 | 1.00 | 16.83 | A |
| ATOM | 4807 | CG | PRO | A | 609 | 36.923 | 89.270 | −29.305 | 1.00 | 16.57 | A |
| ATOM | 4808 | C | PRO | A | 609 | 38.648 | 91.279 | −27.784 | 1.00 | 17.16 | A |
| ATOM | 4809 | O | PRO | A | 609 | 38.322 | 92.003 | −28.725 | 1.00 | 18.05 | A |
| ATOM | 4810 | N | GLN | A | 610 | 39.789 | 91.408 | −27.118 | 1.00 | 17.75 | A |
| ATOM | 4811 | CA | GLN | A | 610 | 40.785 | 92.408 | −27.464 | 1.00 | 19.28 | A |
| ATOM | 4812 | CB | GLN | A | 610 | 41.057 | 93.346 | −26.285 | 1.00 | 22.02 | A |
| ATOM | 4813 | CG | GLN | A | 610 | 39.869 | 94.195 | −25.867 | 1.00 | 26.25 | A |
| ATOM | 4814 | CD | GLN | A | 610 | 40.254 | 95.277 | −24.875 | 1.00 | 28.59 | A |
| ATOM | 4815 | OE1 | GLN | A | 610 | 40.799 | 94.992 | −23.808 | 1.00 | 30.20 | A |
| ATOM | 4816 | NE2 | GLN | A | 610 | 39.973 | 96.527 | −25.225 | 1.00 | 30.13 | A |
| ATOM | 4817 | C | GLN | A | 610 | 42.048 | 91.633 | −27.802 | 1.00 | 18.53 | A |
| ATOM | 4818 | O | GLN | A | 610 | 42.391 | 90.671 | −27.116 | 1.00 | 17.85 | A |
| ATOM | 4819 | N | GLY | A | 611 | 42.732 | 92.043 | −28.863 | 1.00 | 18.19 | A |
| ATOM | 4820 | CA | GLY | A | 611 | 43.945 | 91.355 | −29.257 | 1.00 | 18.07 | A |
| ATOM | 4821 | C | GLY | A | 611 | 45.190 | 92.105 | −28.840 | 1.00 | 18.33 | A |
| ATOM | 4822 | O | GLY | A | 611 | 45.206 | 93.335 | −28.816 | 1.00 | 18.55 | A |
| ATOM | 4823 | N | SER | A | 612 | 46.236 | 91.362 | −28.498 | 1.00 | 18.22 | A |
| ATOM | 4824 | CA | SER | A | 612 | 47.497 | 91.969 | −28.091 | 1.00 | 18.50 | A |
| ATOM | 4825 | CB | SER | A | 612 | 48.366 | 90.941 | −27.365 | 1.00 | 17.71 | A |
| ATOM | 4826 | OG | SER | A | 612 | 49.646 | 91.474 | −27.077 | 1.00 | 17.75 | A |
| ATOM | 4827 | C | SER | A | 612 | 48.247 | 92.494 | −29.308 | 1.00 | 18.79 | A |
| ATOM | 4828 | O | SER | A | 612 | 48.194 | 91.904 | −30.385 | 1.00 | 18.12 | A |
| ATOM | 4829 | N | THR | A | 613 | 48.951 | 93.608 | −29.134 | 1.00 | 20.66 | A |
| ATOM | 4830 | CA | THR | A | 613 | 49.713 | 94.193 | −30.229 | 1.00 | 22.22 | A |
| ATOM | 4831 | CB | THR | A | 613 | 49.428 | 95.704 | −30.370 | 1.00 | 23.24 | A |
| ATOM | 4832 | OG1 | THR | A | 613 | 49.860 | 96.385 | −29.187 | 1.00 | 23.40 | A |
| ATOM | 4833 | CG2 | THR | A | 613 | 47.939 | 95.952 | −30.571 | 1.00 | 22.92 | A |
| ATOM | 4834 | C | THR | A | 613 | 51.212 | 94.001 | −30.007 | 1.00 | 22.80 | A |
| ATOM | 4835 | O | THR | A | 613 | 52.030 | 94.545 | −30.749 | 1.00 | 23.76 | A |
| ATOM | 4836 | N | THR | A | 614 | 51.568 | 93.216 | −28.992 | 1.00 | 22.98 | A |
| ATOM | 4837 | CA | THR | A | 614 | 52.974 | 92.972 | −28.672 | 1.00 | 23.21 | A |
| ATOM | 4838 | CB | THR | A | 614 | 53.417 | 93.818 | −27.469 | 1.00 | 23.94 | A |
| ATOM | 4839 | OG1 | THR | A | 614 | 52.563 | 93.541 | −26.352 | 1.00 | 24.76 | A |
| ATOM | 4840 | CG2 | THR | A | 614 | 53.353 | 95.302 | −27.803 | 1.00 | 25.13 | A |
| ATOM | 4841 | C | THR | A | 614 | 53.288 | 91.514 | −28.349 | 1.00 | 22.57 | A |
| ATOM | 4842 | O | THR | A | 614 | 54.453 | 91.130 | −28.253 | 1.00 | 22.85 | A |
| ATOM | 4843 | N | LYS | A | 615 | 52.247 | 90.709 | −28.176 | 1.00 | 21.12 | A |
| ATOM | 4844 | CA | LYS | A | 615 | 52.404 | 89.297 | −27.849 | 1.00 | 20.03 | A |
| ATOM | 4845 | CB | LYS | A | 615 | 51.862 | 89.040 | −26.439 | 1.00 | 22.00 | A |
| ATOM | 4846 | CG | LYS | A | 615 | 51.894 | 87.593 | −25.987 | 1.00 | 24.67 | A |
| ATOM | 4847 | CD | LYS | A | 615 | 51.077 | 87.399 | −24.711 | 1.00 | 25.80 | A |
| ATOM | 4848 | CE | LYS | A | 615 | 51.569 | 88.291 | −23.581 | 1.00 | 25.99 | A |
| ATOM | 4849 | NZ | LYS | A | 615 | 50.774 | 88.097 | −22.330 | 1.00 | 26.04 | A |
| ATOM | 4850 | C | LYS | A | 615 | 51.629 | 88.463 | −28.863 | 1.00 | 18.41 | A |
| ATOM | 4851 | O | LYS | A | 615 | 50.534 | 88.846 | −29.270 | 1.00 | 17.64 | A |
| ATOM | 4852 | N | TYR | A | 616 | 52.194 | 87.328 | −29.271 | 1.00 | 16.73 | A |
| ATOM | 4853 | CA | TYR | A | 616 | 51.533 | 86.452 | −30.236 | 1.00 | 15.58 | A |
| ATOM | 4854 | CB | TYR | A | 616 | 52.100 | 86.679 | −31.641 | 1.00 | 15.83 | A |
| ATOM | 4855 | CG | TYR | A | 616 | 52.139 | 88.135 | −32.028 | 1.00 | 18.36 | A |
| ATOM | 4856 | CD1 | TYR | A | 616 | 53.209 | 88.945 | −31.651 | 1.00 | 18.36 | A |
| ATOM | 4857 | CE1 | TYR | A | 616 | 53.216 | 90.304 | −31.937 | 1.00 | 20.30 | A |
| ATOM | 4858 | CD2 | TYR | A | 616 | 51.074 | 88.722 | −32.709 | 1.00 | 18.83 | A |
| ATOM | 4859 | CE2 | TYR | A | 616 | 51.069 | 90.080 | −33.000 | 1.00 | 20.65 | A |
| ATOM | 4860 | CZ | TYR | A | 616 | 52.142 | 90.864 | −32.610 | 1.00 | 20.53 | A |
| ATOM | 4861 | OH | TYR | A | 616 | 52.141 | 92.210 | −32.890 | 1.00 | 23.34 | A |
| ATOM | 4862 | C | TYR | A | 616 | 51.707 | 84.992 | −29.847 | 1.00 | 14.72 | A |
| ATOM | 4863 | O | TYR | A | 616 | 52.623 | 84.652 | −29.102 | 1.00 | 13.98 | A |
| ATOM | 4864 | N | ARG | A | 617 | 50.821 | 84.134 | −30.347 | 1.00 | 13.93 | A |
| ATOM | 4865 | CA | ARG | A | 617 | 50.896 | 82.705 | −30.049 | 1.00 | 13.86 | A |
| ATOM | 4866 | CB | ARG | A | 617 | 49.503 | 82.116 | −29.800 | 1.00 | 15.40 | A |
| ATOM | 4867 | CG | ARG | A | 617 | 48.803 | 82.533 | −28.527 | 1.00 | 16.68 | A |
| ATOM | 4868 | CD | ARG | A | 617 | 47.417 | 81.885 | −28.463 | 1.00 | 17.77 | A |
| ATOM | 4869 | NE | ARG | A | 617 | 47.486 | 80.424 | −28.441 | 1.00 | 17.57 | A |
| ATOM | 4870 | CZ | ARG | A | 617 | 46.805 | 79.622 | −29.258 | 1.00 | 17.88 | A |
| ATOM | 4871 | NH1 | ARG | A | 617 | 45.993 | 80.130 | −30.176 | 1.00 | 17.64 | A |
| ATOM | 4872 | NH2 | ARG | A | 617 | 46.941 | 78.307 | −29.165 | 1.00 | 17.23 | A |
| ATOM | 4873 | C | ARG | A | 617 | 51.497 | 81.932 | −31.210 | 1.00 | 13.44 | A |
| ATOM | 4874 | O | ARG | A | 617 | 51.022 | 82.049 | −32.337 | 1.00 | 14.13 | A |
| ATOM | 4875 | N | ILE | A | 618 | 52.544 | 81.154 | −30.953 | 1.00 | 11.96 | A |
| ATOM | 4876 | CA | ILE | A | 618 | 53.097 | 80.331 | −32.016 | 1.00 | 11.92 | A |
| ATOM | 4877 | CB | ILE | A | 618 | 54.629 | 80.488 | −32.179 | 1.00 | 11.58 | A |
| ATOM | 4878 | CG2 | ILE | A | 618 | 55.359 | 80.077 | −30.910 | 1.00 | 12.31 | A |
| ATOM | 4879 | CG1 | ILE | A | 618 | 55.083 | 79.644 | −33.375 | 1.00 | 12.90 | A |
| ATOM | 4880 | CD1 | ILE | A | 618 | 56.396 | 80.078 | −33.976 | 1.00 | 13.57 | A |
| ATOM | 4881 | C | ILE | A | 618 | 52.721 | 78.900 | −31.651 | 1.00 | 11.14 | A |
| ATOM | 4882 | O | ILE | A | 618 | 52.845 | 78.490 | −30.495 | 1.00 | 11.79 | A |
| ATOM | 4883 | N | ILE | A | 619 | 52.238 | 78.162 | −32.643 | 1.00 | 11.17 | A |
| ATOM | 4884 | CA | ILE | A | 619 | 51.767 | 76.791 | −32.466 | 1.00 | 11.40 | A |
| ATOM | 4885 | CB | ILE | A | 619 | 50.259 | 76.713 | −32.779 | 1.00 | 12.11 | A |
| ATOM | 4886 | CG2 | ILE | A | 619 | 49.728 | 75.327 | −32.455 | 1.00 | 14.03 | A |
| ATOM | 4887 | CG1 | ILE | A | 619 | 49.507 | 77.804 | −32.017 | 1.00 | 13.46 | A |
| ATOM | 4888 | CD1 | ILE | A | 619 | 48.191 | 78.193 | −32.670 | 1.00 | 13.83 | A |
| ATOM | 4889 | C | ILE | A | 619 | 52.468 | 75.823 | −33.409 | 1.00 | 11.42 | A |
| ATOM | 4890 | O | ILE | A | 619 | 52.636 | 76.113 | −34.594 | 1.00 | 11.66 | A |
| ATOM | 4891 | N | PHE | A | 620 | 52.864 | 74.665 | −32.895 | 1.00 | 10.46 | A |
| ATOM | 4892 | CA | PHE | A | 620 | 53.509 | 73.672 | −33.742 | 1.00 | 9.92 | A |
| ATOM | 4893 | CB | PHE | A | 620 | 54.987 | 74.019 | −33.964 | 1.00 | 9.89 | A |
| ATOM | 4894 | CG | PHE | A | 620 | 55.842 | 73.914 | −32.734 | 1.00 | 8.70 | A |
| ATOM | 4895 | CD1 | PHE | A | 620 | 56.487 | 72.719 | −32.415 | 1.00 | 10.02 | A |
| ATOM | 4896 | CD2 | PHE | A | 620 | 56.025 | 75.017 | −31.906 | 1.00 | 9.02 | A |
| ATOM | 4897 | CE1 | PHE | A | 620 | 57.309 | 72.627 | −31.289 | 1.00 | 9.45 | A |
| ATOM | 4898 | CE2 | PHE | A | 620 | 56.843 | 74.934 | −30.778 | 1.00 | 10.10 | A |
| ATOM | 4899 | CZ | PHE | A | 620 | 57.486 | 73.737 | −30.471 | 1.00 | 10.05 | A |
| ATOM | 4900 | C | PHE | A | 620 | 53.362 | 72.290 | −33.133 | 1.00 | 10.23 | A |
| ATOM | 4901 | O | PHE | A | 620 | 53.096 | 72.152 | −31.938 | 1.00 | 10.39 | A |
| ATOM | 4902 | N | LYS | A | 621 | 53.519 | 71.268 | −33.963 | 1.00 | 10.49 | A |
| ATOM | 4903 | CA | LYS | A | 621 | 53.382 | 69.900 | −33.498 | 1.00 | 11.17 | A |
| ATOM | 4904 | CB | LYS | A | 621 | 52.896 | 69.007 | −34.649 | 1.00 | 12.49 | A |
| ATOM | 4905 | CG | LYS | A | 621 | 52.656 | 67.559 | −34.247 | 1.00 | 14.18 | A |
| ATOM | 4906 | CD | LYS | A | 621 | 51.816 | 66.822 | −35.284 | 1.00 | 16.44 | A |
| ATOM | 4907 | CE | LYS | A | 621 | 52.483 | 66.793 | −36.648 | 1.00 | 17.70 | A |
| ATOM | 4908 | NZ | LYS | A | 621 | 51.667 | 66.023 | −37.636 | 1.00 | 19.59 | A |
| ATOM | 4909 | C | LYS | A | 621 | 54.685 | 69.362 | −32.926 | 1.00 | 10.69 | A |
| ATOM | 4910 | O | LYS | A | 621 | 55.711 | 69.322 | −33.605 | 1.00 | 11.83 | A |
| ATOM | 4911 | N | ALA | A | 622 | 54.648 | 68.972 | −31.656 | 1.00 | 10.33 | A |
| ATOM | 4912 | CA | ALA | A | 622 | 55.825 | 68.413 | −31.008 | 1.00 | 9.91 | A |
| ATOM | 4913 | CB | ALA | A | 622 | 55.932 | 68.924 | −29.568 | 1.00 | 9.70 | A |
| ATOM | 4914 | C | ALA | A | 622 | 55.684 | 66.896 | −31.012 | 1.00 | 10.39 | A |
| ATOM | 4915 | O | ALA | A | 622 | 54.596 | 66.371 | −30.780 | 1.00 | 11.60 | A |
| ATOM | 4916 | N | ARG | A | 623 | 56.777 | 66.201 | −31.308 | 1.00 | 10.44 | A |
| ATOM | 4917 | CA | ARG | A | 623 | 56.792 | 64.741 | −31.313 | 1.00 | 11.01 | A |
| ATOM | 4918 | CB | ARG | A | 623 | 57.229 | 64.207 | −32.680 | 1.00 | 12.30 | A |
| ATOM | 4919 | CG | ARG | A | 623 | 57.263 | 62.690 | −32.746 | 1.00 | 14.39 | A |
| ATOM | 4920 | CD | ARG | A | 623 | 57.233 | 62.180 | −34.181 | 1.00 | 16.76 | A |
| ATOM | 4921 | NE | ARG | A | 623 | 57.497 | 60.744 | −34.244 | 1.00 | 18.23 | A |
| ATOM | 4922 | CZ | ARG | A | 623 | 58.706 | 60.204 | −34.134 | 1.00 | 19.11 | A |
| ATOM | 4923 | NH1 | ARG | A | 623 | 59.771 | 60.976 | −33.964 | 1.00 | 20.24 | A |
| ATOM | 4924 | NH2 | ARG | A | 623 | 58.851 | 58.889 | −34.182 | 1.00 | 20.27 | A |
| ATOM | 4925 | C | ARG | A | 623 | 57.795 | 64.364 | −30.232 | 1.00 | 10.32 | A |
| ATOM | 4926 | O | ARG | A | 623 | 58.981 | 64.682 | −30.334 | 1.00 | 10.86 | A |
| ATOM | 4927 | N | VAL | A | 624 | 57.306 | 63.687 | −29.197 | 1.00 | 9.90 | A |
| ATOM | 4928 | CA | VAL | A | 624 | 58.121 | 63.333 | −28.040 | 1.00 | 9.26 | A |
| ATOM | 4929 | CB | VAL | A | 624 | 57.547 | 64.045 | −26.794 | 1.00 | 8.57 | A |
| ATOM | 4930 | CG1 | VAL | A | 624 | 58.547 | 64.015 | −25.651 | 1.00 | 8.67 | A |
| ATOM | 4931 | CG2 | VAL | A | 624 | 57.158 | 65.476 | −27.160 | 1.00 | 9.69 | A |
| ATOM | 4932 | C | VAL | A | 624 | 58.191 | 61.831 | −27.770 | 1.00 | 9.00 | A |
| ATOM | 4933 | O | VAL | A | 624 | 57.189 | 61.130 | −27.868 | 1.00 | 9.09 | A |
| ATOM | 4934 | N | PRO | A | 625 | 59.381 | 61.325 | −27.401 | 1.00 | 9.44 | A |
| ATOM | 4935 | CD | PRO | A | 625 | 60.665 | 62.037 | −27.290 | 1.00 | 10.05 | A |
| ATOM | 4936 | CA | PRO | A | 625 | 59.555 | 59.895 | −27.121 | 1.00 | 9.16 | A |
| ATOM | 4937 | CB | PRO | A | 625 | 61.054 | 59.765 | −26.851 | 1.00 | 10.20 | A |
| ATOM | 4938 | CG | PRO | A | 625 | 61.654 | 60.942 | −27.563 | 1.00 | 10.52 | A |
| ATOM | 4939 | C | PRO | A | 625 | 58.754 | 59.432 | −25.911 | 1.00 | 9.12 | A |
| ATOM | 4940 | O | PRO | A | 625 | 58.307 | 60.249 | −25.100 | 1.00 | 8.82 | A |
| ATOM | 4941 | N | PRO | A | 626 | 58.571 | 58.111 | −25.769 | 1.00 | 9.36 | A |
| ATOM | 4942 | CD | PRO | A | 626 | 59.055 | 57.027 | −26.644 | 1.00 | 9.11 | A |
| ATOM | 4943 | CA | PRO | A | 626 | 57.824 | 57.575 | −24.628 | 1.00 | 8.73 | A |
| ATOM | 4944 | CB | PRO | A | 626 | 57.916 | 56.063 | −24.830 | 1.00 | 8.19 | A |
| ATOM | 4945 | CG | PRO | A | 626 | 58.096 | 55.913 | −26.329 | 1.00 | 9.53 | A |
| ATOM | 4946 | C | PRO | A | 626 | 58.566 | 58.008 | −23.364 | 1.00 | 8.14 | A |
| ATOM | 4947 | O | PRO | A | 626 | 59.786 | 57.847 | −23.286 | 1.00 | 9.15 | A |
| ATOM | 4948 | N | MSE | A | 627 | 57.847 | 58.564 | −22.390 | 1.00 | 7.51 | A |
| ATOM | 4949 | CA | MSE | A | 627 | 58.460 | 59.002 | −21.130 | 1.00 | 8.21 | A |
| ATOM | 4950 | CB | MSE | A | 627 | 58.747 | 57.783 | −20.250 | 1.00 | 10.53 | A |
| ATOM | 4951 | CG | MSE | A | 627 | 57.500 | 56.983 | −19.914 | 1.00 | 10.63 | A |
| ATOM | 4952 | SE | MSE | A | 627 | 57.871 | 55.262 | −19.133 | 1.00 | 20.00 | A |
| ATOM | 4953 | CE | MSE | A | 627 | 58.495 | 55.837 | −17.415 | 1.00 | 15.15 | A |
| ATOM | 4954 | C | MSE | A | 627 | 59.755 | 59.755 | −21.414 | 1.00 | 8.18 | A |
| ATOM | 4955 | O | MSE | A | 627 | 60.759 | 59.584 | −20.712 | 1.00 | 7.85 | A |
| ATOM | 4956 | N | GLY | A | 628 | 59.718 | 60.602 | −22.442 | 1.00 | 8.18 | A |
| ATOM | 4957 | CA | GLY | A | 628 | 60.908 | 61.329 | −22.841 | 1.00 | 8.72 | A |
| ATOM | 4958 | C | GLY | A | 628 | 60.819 | 62.826 | −23.028 | 1.00 | 8.56 | A |
| ATOM | 4959 | O | GLY | A | 628 | 59.847 | 63.470 | −22.627 | 1.00 | 7.33 | A |
| ATOM | 4960 | N | LEU | A | 629 | 61.853 | 63.366 | −23.663 | 1.00 | 7.72 | A |
| ATOM | 4961 | CA | LEU | A | 629 | 61.972 | 64.797 | −23.912 | 1.00 | 8.61 | A |
| ATOM | 4962 | CB | LEU | A | 629 | 63.027 | 65.391 | −22.979 | 1.00 | 8.59 | A |
| ATOM | 4963 | CG | LEU | A | 629 | 62.778 | 65.233 | −21.480 | 1.00 | 9.21 | A |
| ATOM | 4964 | CD1 | LEU | A | 629 | 64.046 | 65.560 | −20.708 | 1.00 | 9.45 | A |
| ATOM | 4965 | CD2 | LEU | A | 629 | 61.632 | 66.140 | −21.064 | 1.00 | 9.65 | A |
| ATOM | 4966 | C | LEU | A | 629 | 62.382 | 65.079 | −25.347 | 1.00 | 8.71 | A |
| ATOM | 4967 | O | LEU | A | 629 | 63.088 | 64.284 | −25.966 | 1.00 | 9.28 | A |
| ATOM | 4968 | N | ALA | A | 630 | 61.931 | 66.212 | −25.871 | 1.00 | 8.74 | A |
| ATOM | 4969 | CA | ALA | A | 630 | 62.276 | 66.625 | −27.229 | 1.00 | 9.15 | A |
| ATOM | 4970 | CB | ALA | A | 630 | 61.157 | 66.286 | −28.197 | 1.00 | 9.76 | A |
| ATOM | 4971 | C | ALA | A | 630 | 62.514 | 68.126 | −27.201 | 1.00 | 9.28 | A |
| ATOM | 4972 | O | ALA | A | 630 | 61.702 | 68.884 | −26.663 | 1.00 | 8.90 | A |
| ATOM | 4973 | N | THR | A | 631 | 63.632 | 68.548 | −27.784 | 1.00 | 9.16 | A |
| ATOM | 4974 | CA | THR | A | 631 | 64.020 | 69.955 | −27.810 | 1.00 | 9.71 | A |
| ATOM | 4975 | CB | THR | A | 631 | 65.524 | 70.096 | −27.498 | 1.00 | 10.27 | A |
| ATOM | 4976 | OG1 | THR | A | 631 | 65.823 | 69.394 | −26.284 | 1.00 | 10.07 | A |
| ATOM | 4977 | CG2 | THR | A | 631 | 65.914 | 71.559 | −27.344 | 1.00 | 10.84 | A |
| ATOM | 4978 | C | THR | A | 631 | 63.754 | 70.623 | −29.155 | 1.00 | 9.81 | A |
| ATOM | 4979 | O | THR | A | 631 | 64.015 | 70.038 | −30.205 | 1.00 | 10.56 | A |
| ATOM | 4980 | N | TYR | A | 632 | 63.226 | 71.844 | −29.108 | 1.00 | 9.56 | A |
| ATOM | 4981 | CA | TYR | A | 632 | 62.958 | 72.628 | −30.310 | 1.00 | 9.78 | A |
| ATOM | 4982 | CB | TYR | A | 632 | 61.457 | 72.724 | −30.597 | 1.00 | 10.51 | A |
| ATOM | 4983 | CG | TYR | A | 632 | 60.827 | 71.415 | −31.011 | 1.00 | 10.25 | A |
| ATOM | 4984 | CD1 | TYR | A | 632 | 60.461 | 70.466 | −30.058 | 1.00 | 9.79 | A |
| ATOM | 4985 | CE1 | TYR | A | 632 | 59.891 | 69.256 | −30.432 | 1.00 | 11.56 | A |
| ATOM | 4986 | CD2 | TYR | A | 632 | 60.608 | 71.116 | −32.356 | 1.00 | 9.93 | A |
| ATOM | 4987 | CE2 | TYR | A | 632 | 60.042 | 69.906 | −32.743 | 1.00 | 11.41 | A |
| ATOM | 4988 | CZ | TYR | A | 632 | 59.686 | 68.981 | −31.774 | 1.00 | 10.98 | A |
| ATOM | 4989 | OH | TYR | A | 632 | 59.133 | 67.779 | −32.152 | 1.00 | 12.83 | A |
| ATOM | 4990 | C | TYR | A | 632 | 63.528 | 74.027 | −30.123 | 1.00 | 9.80 | A |
| ATOM | 4991 | O | TYR | A | 632 | 63.821 | 74.449 | −29.001 | 1.00 | 9.66 | A |
| ATOM | 4992 | N | VAL | A | 633 | 63.680 | 74.749 | −31.227 | 1.00 | 9.77 | A |
| ATOM | 4993 | CA | VAL | A | 633 | 64.226 | 76.096 | −31.188 | 1.00 | 10.76 | A |
| ATOM | 4994 | CB | VAL | A | 633 | 65.618 | 76.152 | −31.867 | 1.00 | 11.04 | A |
| ATOM | 4995 | CG1 | VAL | A | 633 | 66.193 | 77.557 | −31.768 | 1.00 | 13.11 | A |
| ATOM | 4996 | CG2 | VAL | A | 633 | 66.556 | 75.144 | −31.225 | 1.00 | 12.71 | A |
| ATOM | 4997 | C | VAL | A | 633 | 63.304 | 77.076 | −31.906 | 1.00 | 10.49 | A |
| ATOM | 4998 | O | VAL | A | 633 | 62.811 | 76.786 | −32.995 | 1.00 | 10.92 | A |
| ATOM | 4999 | N | LEU | A | 634 | 63.060 | 78.220 | −31.277 | 1.00 | 11.11 | A |
| ATOM | 5000 | CA | LEU | A | 634 | 62.227 | 79.265 | −31.866 | 1.00 | 12.07 | A |
| ATOM | 5001 | CB | LEU | A | 634 | 61.213 | 79.800 | −30.853 | 1.00 | 12.58 | A |
| ATOM | 5002 | CG | LEU | A | 634 | 60.244 | 78.792 | −30.237 | 1.00 | 15.79 | A |
| ATOM | 5003 | CD1 | LEU | A | 634 | 59.242 | 79.541 | −29.371 | 1.00 | 15.32 | A |
| ATOM | 5004 | CD2 | LEU | A | 634 | 59.527 | 78.022 | −31.322 | 1.00 | 17.18 | A |
| ATOM | 5005 | C | LEU | A | 634 | 63.181 | 80.379 | −32.269 | 1.00 | 12.31 | A |
| ATOM | 5006 | O | LEU | A | 634 | 63.947 | 80.872 | −31.443 | 1.00 | 12.05 | A |
| ATOM | 5007 | N | THR | A | 635 | 63.135 | 80.768 | −33.540 | 1.00 | 12.28 | A |
| ATOM | 5008 | CA | THR | A | 635 | 64.018 | 81.804 | −34.059 | 1.00 | 13.14 | A |
| ATOM | 5009 | CB | THR | A | 635 | 64.983 | 81.207 | −35.099 | 1.00 | 12.77 | A |
| ATOM | 5010 | OG1 | THR | A | 635 | 65.667 | 80.086 | −34.522 | 1.00 | 13.31 | A |
| ATOM | 5011 | CG2 | THR | A | 635 | 66.004 | 82.244 | −35.542 | 1.00 | 12.18 | A |
| ATOM | 5012 | C | THR | A | 635 | 63.238 | 82.943 | −34.706 | 1.00 | 13.84 | A |
| ATOM | 5013 | O | THR | A | 635 | 62.285 | 82.718 | −35.449 | 1.00 | 13.21 | A |
| ATOM | 5014 | N | ILE | A | 636 | 63.655 | 84.171 | −34.424 | 1.00 | 15.47 | A |
| ATOM | 5015 | CA | ILE | A | 636 | 62.982 | 85.334 | −34.985 | 1.00 | 16.90 | A |
| ATOM | 5016 | CB | ILE | A | 636 | 63.064 | 86.544 | −34.022 | 1.00 | 17.25 | A |
| ATOM | 5017 | CG2 | ILE | A | 636 | 64.492 | 87.069 | −33.958 | 1.00 | 17.16 | A |
| ATOM | 5018 | CG1 | ILE | A | 636 | 62.120 | 87.653 | −34.495 | 1.00 | 16.98 | A |
| ATOM | 5019 | CD1 | ILE | A | 636 | 62.027 | 88.831 | −33.538 | 1.00 | 17.62 | A |
| ATOM | 5020 | C | ILE | A | 636 | 63.610 | 85.712 | −36.321 | 1.00 | 18.42 | A |
| ATOM | 5021 | O | ILE | A | 636 | 64.781 | 85.430 | −36.570 | 1.00 | 17.70 | A |
| ATOM | 5022 | N | SER | A | 637 | 62.811 | 86.330 | −37.184 | 1.00 | 20.09 | A |
| ATOM | 5023 | CA | SER | A | 637 | 63.275 | 86.780 | −38.492 | 1.00 | 22.79 | A |
| ATOM | 5024 | CB | SER | A | 637 | 62.850 | 85.797 | −39.587 | 1.00 | 23.81 | A |
| ATOM | 5025 | OG | SER | A | 637 | 61.442 | 85.665 | −39.645 | 1.00 | 26.10 | A |
| ATOM | 5026 | C | SER | A | 637 | 62.651 | 88.149 | −38.739 | 1.00 | 24.15 | A |
| ATOM | 5027 | O | SER | A | 637 | 61.724 | 88.545 | −38.035 | 1.00 | 23.27 | A |
| ATOM | 5028 | N | ASP | A | 638 | 63.160 | 88.874 | −39.730 | 1.00 | 26.58 | A |
| ATOM | 5029 | CA | ASP | A | 638 | 62.641 | 90.203 | −40.034 | 1.00 | 28.99 | A |
| ATOM | 5030 | CB | ASP | A | 638 | 63.643 | 90.978 | −40.897 | 1.00 | 31.15 | A |
| ATOM | 5031 | CG | ASP | A | 638 | 63.893 | 90.317 | −42.240 | 1.00 | 33.16 | A |
| ATOM | 5032 | OD1 | ASP | A | 638 | 62.934 | 90.185 | −43.030 | 1.00 | 34.91 | A |
| ATOM | 5033 | OD2 | ASP | A | 638 | 65.050 | 89.930 | −42.507 | 1.00 | 34.82 | A |
| ATOM | 5034 | C | ASP | A | 638 | 61.289 | 90.154 | −40.737 | 1.00 | 29.32 | A |
| ATOM | 5035 | O | ASP | A | 638 | 60.477 | 91.070 | −40.599 | 1.00 | 30.07 | A |
| ATOM | 5036 | N | SER | A | 639 | 61.046 | 89.080 | −41.482 | 1.00 | 28.95 | A |
| ATOM | 5037 | CA | SER | A | 639 | 59.793 | 88.930 | −42.210 | 1.00 | 28.76 | A |
| ATOM | 5038 | CB | SER | A | 639 | 60.020 | 89.203 | −43.699 | 1.00 | 28.80 | A |
| ATOM | 5039 | OG | SER | A | 639 | 60.995 | 88.324 | −44.232 | 1.00 | 29.52 | A |
| ATOM | 5040 | C | SER | A | 639 | 59.192 | 87.542 | −42.029 | 1.00 | 28.41 | A |
| ATOM | 5041 | O | SER | A | 639 | 59.794 | 86.670 | −41.403 | 1.00 | 27.60 | A |
| ATOM | 5042 | N | LYS | A | 640 | 58.002 | 87.343 | −42.586 | 1.00 | 27.73 | A |
| ATOM | 5043 | CA | LYS | A | 640 | 57.315 | 86.063 | −42.481 | 1.00 | 27.72 | A |
| ATOM | 5044 | CB | LYS | A | 640 | 56.025 | 86.081 | −43.305 | 1.00 | 29.03 | A |
| ATOM | 5045 | CG | LYS | A | 640 | 55.001 | 87.094 | −42.820 | 1.00 | 31.44 | A |
| ATOM | 5046 | CD | LYS | A | 640 | 53.668 | 86.941 | −43.539 | 1.00 | 32.89 | A |
| ATOM | 5047 | CE | LYS | A | 640 | 53.799 | 87.189 | −45.032 | 1.00 | 33.76 | A |
| ATOM | 5048 | NZ | LYS | A | 640 | 52.485 | 87.054 | −45.718 | 1.00 | 34.46 | A |
| ATOM | 5049 | C | LYS | A | 640 | 58.186 | 84.897 | −42.928 | 1.00 | 26.49 | A |
| ATOM | 5050 | O | LYS | A | 640 | 58.627 | 84.839 | −44.076 | 1.00 | 26.60 | A |
| ATOM | 5051 | N | PRO | A | 641 | 58.453 | 83.950 | −42.014 | 1.00 | 25.24 | A |
| ATOM | 5052 | CD | PRO | A | 641 | 58.068 | 83.956 | −40.592 | 1.00 | 25.03 | A |
| ATOM | 5053 | CA | PRO | A | 641 | 59.275 | 82.780 | −42.327 | 1.00 | 24.06 | A |
| ATOM | 5054 | CB | PRO | A | 641 | 59.570 | 82.191 | −40.951 | 1.00 | 24.74 | A |
| ATOM | 5055 | CG | PRO | A | 641 | 58.346 | 82.531 | −40.181 | 1.00 | 25.06 | A |
| ATOM | 5056 | C | PRO | A | 641 | 58.544 | 81.804 | −43.246 | 1.00 | 23.11 | A |
| ATOM | 5057 | O | PRO | A | 641 | 57.314 | 81.732 | −43.250 | 1.00 | 22.10 | A |
| ATOM | 5058 | N | GLU | A | 642 | 59.316 | 81.052 | −44.019 | 1.00 | 22.60 | A |
| ATOM | 5059 | CA | GLU | A | 642 | 58.772 | 80.090 | −44.969 | 1.00 | 22.59 | A |
| ATOM | 5060 | CB | GLU | A | 642 | 59.920 | 79.413 | −45.725 | 1.00 | 24.75 | A |
| ATOM | 5061 | CG | GLU | A | 642 | 59.482 | 78.292 | −46.655 | 1.00 | 27.41 | A |
| ATOM | 5062 | CD | GLU | A | 642 | 60.650 | 77.630 | −47.364 | 1.00 | 29.34 | A |
| ATOM | 5063 | OE1 | GLU | A | 642 | 60.415 | 76.662 | −48.118 | 1.00 | 30.70 | A |
| ATOM | 5064 | OE2 | GLU | A | 642 | 61.801 | 78.077 | −47.167 | 1.00 | 30.38 | A |
| ATOM | 5065 | C | GLU | A | 642 | 57.863 | 79.011 | −44.387 | 1.00 | 21.56 | A |
| ATOM | 5066 | O | GLU | A | 642 | 56.864 | 78.641 | −45.001 | 1.00 | 22.04 | A |
| ATOM | 5067 | N | HIS | A | 643 | 58.202 | 78.511 | −43.204 | 1.00 | 20.18 | A |
| ATOM | 5068 | CA | HIS | A | 643 | 57.427 | 77.434 | −42.597 | 1.00 | 18.61 | A |
| ATOM | 5069 | CB | HIS | A | 643 | 58.391 | 76.385 | −42.044 | 1.00 | 19.22 | A |
| ATOM | 5070 | CG | HIS | A | 643 | 59.281 | 75.789 | −43.088 | 1.00 | 19.81 | A |
| ATOM | 5071 | CD2 | HIS | A | 643 | 60.558 | 76.071 | −43.437 | 1.00 | 19.90 | A |
| ATOM | 5072 | ND1 | HIS | A | 643 | 58.851 | 74.816 | −43.963 | 1.00 | 20.57 | A |
| ATOM | 5073 | CE1 | HIS | A | 643 | 59.825 | 74.523 | −44.806 | 1.00 | 20.30 | A |
| ATOM | 5074 | NE2 | HIS | A | 643 | 60.871 | 75.272 | −44.509 | 1.00 | 19.73 | A |
| ATOM | 5075 | C | HIS | A | 643 | 56.421 | 77.828 | −41.527 | 1.00 | 17.54 | A |
| ATOM | 5076 | O | HIS | A | 643 | 55.958 | 76.978 | −40.764 | 1.00 | 16.42 | A |
| ATOM | 5077 | N | THR | A | 644 | 56.079 | 79.110 | −41.474 | 1.00 | 15.71 | A |
| ATOM | 5078 | CA | THR | A | 644 | 55.110 | 79.598 | −40.503 | 1.00 | 15.08 | A |
| ATOM | 5079 | CB | THR | A | 644 | 55.752 | 80.606 | −39.524 | 1.00 | 14.66 | A |
| ATOM | 5080 | OG1 | THR | A | 644 | 56.816 | 79.964 | −38.808 | 1.00 | 14.87 | A |
| ATOM | 5081 | CG2 | THR | A | 644 | 54.719 | 81.122 | −38.524 | 1.00 | 14.03 | A |
| ATOM | 5082 | C | THR | A | 644 | 53.957 | 80.279 | −41.236 | 1.00 | 15.17 | A |
| ATOM | 5083 | O | THR | A | 644 | 54.177 | 81.138 | −42.092 | 1.00 | 15.67 | A |
| ATOM | 5084 | N | SER | A | 645 | 52.733 | 79.874 | −40.909 | 1.00 | 14.18 | A |
| ATOM | 5085 | CA | SER | A | 645 | 51.535 | 80.448 | −41.514 | 1.00 | 13.57 | A |
| ATOM | 5086 | CB | SER | A | 645 | 50.586 | 79.347 | −41.989 | 1.00 | 14.13 | A |
| ATOM | 5087 | OG | SER | A | 645 | 50.085 | 78.600 | −40.892 | 1.00 | 14.02 | A |
| ATOM | 5088 | C | SER | A | 645 | 50.836 | 81.303 | −40.467 | 1.00 | 13.45 | A |
| ATOM | 5089 | O | SER | A | 645 | 51.140 | 81.213 | −39.276 | 1.00 | 13.19 | A |
| ATOM | 5090 | N | TYR | A | 646 | 49.896 | 82.129 | −40.910 | 1.00 | 13.22 | A |
| ATOM | 5091 | CA | TYR | A | 646 | 49.167 | 83.006 | −40.003 | 1.00 | 13.22 | A |
| ATOM | 5092 | CB | TYR | A | 646 | 49.536 | 84.464 | −40.279 | 1.00 | 14.27 | A |
| ATOM | 5093 | CG | TYR | A | 646 | 51.011 | 84.707 | −40.096 | 1.00 | 14.12 | A |
| ATOM | 5094 | CD1 | TYR | A | 646 | 51.917 | 84.400 | −41.110 | 1.00 | 14.67 | A |
| ATOM | 5095 | CE1 | TYR | A | 646 | 53.287 | 84.520 | −40.910 | 1.00 | 15.68 | A |
| ATOM | 5096 | CD2 | TYR | A | 646 | 51.513 | 85.150 | −38.875 | 1.00 | 14.12 | A |
| ATOM | 5097 | CE2 | TYR | A | 646 | 52.882 | 85.274 | −38.664 | 1.00 | 14.90 | A |
| ATOM | 5098 | CZ | TYR | A | 646 | 53.762 | 84.954 | −39.685 | 1.00 | 14.72 | A |
| ATOM | 5099 | OH | TYR | A | 646 | 55.119 | 85.045 | −39.472 | 1.00 | 16.28 | A |
| ATOM | 5100 | C | TYR | A | 646 | 47.665 | 82.815 | −40.107 | 1.00 | 13.24 | A |
| ATOM | 5101 | O | TYR | A | 646 | 47.098 | 82.807 | −41.198 | 1.00 | 14.84 | A |
| ATOM | 5102 | N | ALA | A | 647 | 47.025 | 82.670 | −38.954 | 1.00 | 12.63 | A |
| ATOM | 5103 | CA | ALA | A | 647 | 45.590 | 82.463 | −38.900 | 1.00 | 12.30 | A |
| ATOM | 5104 | CB | ALA | A | 647 | 45.187 | 82.017 | −37.497 | 1.00 | 12.86 | A |
| ATOM | 5105 | C | ALA | A | 647 | 44.795 | 83.697 | −39.288 | 1.00 | 12.48 | A |
| ATOM | 5106 | O | ALA | A | 647 | 45.230 | 84.832 | −39.085 | 1.00 | 12.90 | A |
| ATOM | 5107 | N | SER | A | 648 | 43.624 | 83.464 | −39.862 | 1.00 | 12.72 | A |
| ATOM | 5108 | CA | SER | A | 648 | 42.742 | 84.556 | −40.223 | 1.00 | 12.98 | A |
| ATOM | 5109 | CB | SER | A | 648 | 41.901 | 84.192 | −41.448 | 1.00 | 14.58 | A |
| ATOM | 5110 | OG | SER | A | 648 | 41.117 | 83.036 | −41.214 | 1.00 | 17.12 | A |
| ATOM | 5111 | C | SER | A | 648 | 41.851 | 84.716 | −38.998 | 1.00 | 12.22 | A |
| ATOM | 5112 | O | SER | A | 648 | 41.676 | 83.770 | −38.222 | 1.00 | 12.30 | A |
| ATOM | 5113 | N | ASN | A | 649 | 41.300 | 85.906 | −38.812 | 1.00 | 11.83 | A |
| ATOM | 5114 | CA | ASN | A | 649 | 40.434 | 86.163 | −37.672 | 1.00 | 11.16 | A |
| ATOM | 5115 | CB | ASN | A | 649 | 41.183 | 86.971 | −36.609 | 1.00 | 11.94 | A |
| ATOM | 5116 | CG | ASN | A | 649 | 42.355 | 86.208 | −36.017 | 1.00 | 12.08 | A |
| ATOM | 5117 | OD1 | ASN | A | 649 | 42.188 | 85.394 | −35.105 | 1.00 | 11.76 | A |
| ATOM | 5118 | ND2 | ASN | A | 649 | 43.547 | 86.456 | −36.544 | 1.00 | 12.65 | A |
| ATOM | 5119 | C | ASN | A | 649 | 39.200 | 86.917 | −38.132 | 1.00 | 12.07 | A |
| ATOM | 5120 | O | ASN | A | 649 | 39.300 | 87.899 | −38.870 | 1.00 | 12.58 | A |
| ATOM | 5121 | N | LEU | A | 650 | 38.041 | 86.445 | −37.692 | 1.00 | 11.67 | A |
| ATOM | 5122 | CA | LEU | A | 650 | 36.763 | 87.046 | −38.053 | 1.00 | 11.94 | A |
| ATOM | 5123 | CB | LEU | A | 650 | 35.995 | 86.099 | −38.976 | 1.00 | 11.82 | A |
| ATOM | 5124 | CG | LEU | A | 650 | 34.550 | 86.449 | −39.334 | 1.00 | 11.91 | A |
| ATOM | 5125 | CD1 | LEU | A | 650 | 34.509 | 87.731 | −40.159 | 1.00 | 12.74 | A |
| ATOM | 5126 | CD2 | LEU | A | 650 | 33.941 | 85.294 | −40.117 | 1.00 | 13.05 | A |
| ATOM | 5127 | C | LEU | A | 650 | 35.947 | 87.314 | −36.796 | 1.00 | 12.84 | A |
| ATOM | 5128 | O | LEU | A | 650 | 35.635 | 86.390 | −36.040 | 1.00 | 12.29 | A |
| ATOM | 5129 | N | LEU | A | 651 | 35.609 | 88.579 | −36.574 | 1.00 | 13.28 | A |
| ATOM | 5130 | CA | LEU | A | 651 | 34.828 | 88.972 | −35.410 | 1.00 | 13.95 | A |
| ATOM | 5131 | CB | LEU | A | 651 | 35.427 | 90.233 | −34.775 | 1.00 | 15.51 | A |
| ATOM | 5132 | CG | LEU | A | 651 | 34.996 | 90.608 | −33.352 | 1.00 | 17.09 | A |
| ATOM | 5133 | CD1 | LEU | A | 651 | 33.573 | 91.118 | −33.352 | 1.00 | 18.83 | A |
| ATOM | 5134 | CD2 | LEU | A | 651 | 35.144 | 89.403 | −32.434 | 1.00 | 17.37 | A |
| ATOM | 5135 | C | LEU | A | 651 | 33.396 | 89.227 | −35.860 | 1.00 | 14.48 | A |
| ATOM | 5136 | O | LEU | A | 651 | 33.128 | 90.156 | −36.624 | 1.00 | 15.41 | A |
| ATOM | 5137 | N | LEU | A | 652 | 32.481 | 88.391 | −35.383 | 1.00 | 14.89 | A |
| ATOM | 5138 | CA | LEU | A | 652 | 31.079 | 88.497 | −35.747 | 1.00 | 15.05 | A |
| ATOM | 5139 | CB | LEU | A | 652 | 30.513 | 87.104 | −36.041 | 1.00 | 14.75 | A |
| ATOM | 5140 | CG | LEU | A | 652 | 31.240 | 86.327 | −37.142 | 1.00 | 14.59 | A |
| ATOM | 5141 | CD1 | LEU | A | 652 | 30.672 | 84.919 | −37.249 | 1.00 | 14.90 | A |
| ATOM | 5142 | CD2 | LEU | A | 652 | 31.101 | 87.068 | −38.471 | 1.00 | 14.78 | A |
| ATOM | 5143 | C | LEU | A | 652 | 30.230 | 89.179 | −34.684 | 1.00 | 16.72 | A |
| ATOM | 5144 | O | LEU | A | 652 | 30.096 | 88.687 | −33.560 | 1.00 | 15.79 | A |
| ATOM | 5145 | N | ARG | A | 653 | 29.668 | 90.324 | −35.050 | 1.00 | 18.41 | A |
| ATOM | 5146 | CA | ARG | A | 653 | 28.800 | 91.083 | −34.164 | 1.00 | 21.34 | A |
| ATOM | 5147 | CB | ARG | A | 653 | 29.502 | 91.426 | −32.848 | 1.00 | 22.76 | A |
| ATOM | 5148 | CG | ARG | A | 653 | 30.545 | 92.524 | −32.929 | 1.00 | 25.26 | A |
| ATOM | 5149 | CD | ARG | A | 653 | 30.599 | 93.251 | −31.595 | 1.00 | 27.66 | A |
| ATOM | 5150 | NE | ARG | A | 653 | 31.884 | 93.887 | −31.338 | 1.00 | 29.57 | A |
| ATOM | 5151 | CZ | ARG | A | 653 | 32.159 | 94.575 | −30.235 | 1.00 | 30.35 | A |
| ATOM | 5152 | NH1 | ARG | A | 653 | 31.235 | 94.718 | −29.295 | 1.00 | 30.94 | A |
| ATOM | 5153 | NH2 | ARG | A | 653 | 33.361 | 95.108 | −30.064 | 1.00 | 31.04 | A |
| ATOM | 5154 | C | ARG | A | 653 | 28.365 | 92.369 | −34.838 | 1.00 | 22.58 | A |
| ATOM | 5155 | O | ARG | A | 653 | 28.976 | 92.810 | −35.811 | 1.00 | 22.35 | A |
| ATOM | 5156 | N | LYS | A | 654 | 27.300 | 92.961 | −34.314 | 1.00 | 24.79 | A |
| ATOM | 5157 | CA | LYS | A | 654 | 26.787 | 94.214 | −34.841 | 1.00 | 26.87 | A |
| ATOM | 5158 | CB | LYS | A | 654 | 25.297 | 94.350 | −34.518 | 1.00 | 28.87 | A |
| ATOM | 5159 | CG | LYS | A | 654 | 24.403 | 93.357 | −35.251 | 1.00 | 31.28 | A |
| ATOM | 5160 | CD | LYS | A | 654 | 23.517 | 94.059 | −36.275 | 1.00 | 33.37 | A |
| ATOM | 5161 | CE | LYS | A | 654 | 24.341 | 94.806 | −37.315 | 1.00 | 34.26 | A |
| ATOM | 5162 | NZ | LYS | A | 654 | 23.486 | 95.582 | −38.259 | 1.00 | 35.77 | A |
| ATOM | 5163 | C | LYS | A | 654 | 27.567 | 95.349 | −34.186 | 1.00 | 27.13 | A |
| ATOM | 5164 | O | LYS | A | 654 | 28.051 | 95.207 | −33.063 | 1.00 | 27.36 | A |
| ATOM | 5165 | N | ASN | A | 655 | 27.696 | 96.465 | −34.893 | 1.00 | 27.40 | A |
| ATOM | 5166 | CA | ASN | A | 655 | 28.411 | 97.626 | −34.376 | 1.00 | 27.55 | A |
| ATOM | 5167 | CB | ASN | A | 655 | 27.671 | 98.196 | −33.165 | 1.00 | 29.06 | A |
| ATOM | 5168 | CG | ASN | A | 655 | 26.166 | 98.203 | −33.353 | 1.00 | 30.72 | A |
| ATOM | 5169 | OD1 | ASN | A | 655 | 25.651 | 98.755 | −34.327 | 1.00 | 31.94 | A |
| ATOM | 5170 | ND2 | ASN | A | 655 | 25.451 | 97.585 | −32.419 | 1.00 | 31.37 | A |
| ATOM | 5171 | C | ASN | A | 655 | 29.843 | 97.273 | −33.978 | 1.00 | 26.37 | A |
| ATOM | 5172 | O | ASN | A | 655 | 30.257 | 97.502 | −32.841 | 1.00 | 26.45 | A |
| ATOM | 5173 | N | PRO | A | 656 | 30.621 | 96.708 | −34.911 | 1.00 | 25.13 | A |
| ATOM | 5174 | CD | PRO | A | 656 | 30.269 | 96.273 | −36.276 | 1.00 | 24.78 | A |
| ATOM | 5175 | CA | PRO | A | 656 | 32.003 | 96.346 | −34.595 | 1.00 | 23.93 | A |
| ATOM | 5176 | CB | PRO | A | 656 | 32.340 | 95.333 | −35.678 | 1.00 | 23.98 | A |
| ATOM | 5177 | CG | PRO | A | 656 | 31.621 | 95.907 | −36.863 | 1.00 | 24.37 | A |
| ATOM | 5178 | C | PRO | A | 656 | 32.939 | 97.547 | −34.637 | 1.00 | 23.11 | A |
| ATOM | 5179 | O | PRO | A | 656 | 32.640 | 98.561 | −35.267 | 1.00 | 22.26 | A |
| ATOM | 5180 | N | THR | A | 657 | 34.067 | 97.422 | −33.948 | 1.00 | 22.32 | A |
| ATOM | 5181 | CA | THR | A | 657 | 35.079 | 98.466 | −33.930 | 1.00 | 21.57 | A |
| ATOM | 5182 | CB | THR | A | 657 | 35.217 | 99.113 | −32.534 | 1.00 | 21.87 | A |
| ATOM | 5183 | OG1 | THR | A | 657 | 35.326 | 98.092 | −31.536 | 1.00 | 22.67 | A |
| ATOM | 5184 | CG2 | THR | A | 657 | 34.010 | 99.996 | −32.237 | 1.00 | 21.99 | A |
| ATOM | 5185 | C | THR | A | 657 | 36.392 | 97.807 | −34.329 | 1.00 | 21.08 | A |
| ATOM | 5186 | O | THR | A | 657 | 36.543 | 96.589 | −34.210 | 1.00 | 20.93 | A |
| ATOM | 5187 | N | SER | A | 658 | 37.335 | 98.609 | −34.806 | 1.00 | 20.46 | A |
| ATOM | 5188 | CA | SER | A | 658 | 38.626 | 98.099 | −35.249 | 1.00 | 20.63 | A |
| ATOM | 5189 | CB | SER | A | 658 | 39.533 | 99.255 | −35.660 | 1.00 | 20.97 | A |
| ATOM | 5190 | OG | SER | A | 658 | 39.882 | 100.035 | −34.531 | 1.00 | 21.67 | A |
| ATOM | 5191 | C | SER | A | 658 | 39.337 | 97.257 | −34.197 | 1.00 | 20.59 | A |
| ATOM | 5192 | O | SER | A | 658 | 39.126 | 97.426 | −32.997 | 1.00 | 20.25 | A |
| ATOM | 5193 | N | LEU | A | 659 | 40.186 | 96.350 | −34.670 | 1.00 | 20.47 | A |
| ATOM | 5194 | CA | LEU | A | 659 | 40.954 | 95.469 | −33.800 | 1.00 | 21.02 | A |
| ATOM | 5195 | CB | LEU | A | 659 | 40.294 | 94.088 | −33.719 | 1.00 | 21.51 | A |
| ATOM | 5196 | CG | LEU | A | 659 | 38.979 | 93.982 | −32.941 | 1.00 | 21.80 | A |
| ATOM | 5197 | CD1 | LEU | A | 659 | 38.354 | 92.610 | −33.155 | 1.00 | 22.38 | A |
| ATOM | 5198 | CD2 | LEU | A | 659 | 39.243 | 94.224 | −31.466 | 1.00 | 21.76 | A |
| ATOM | 5199 | C | LEU | A | 659 | 42.372 | 95.328 | −34.340 | 1.00 | 21.00 | A |
| ATOM | 5200 | O | LEU | A | 659 | 42.695 | 94.351 | −35.019 | 1.00 | 21.34 | A |
| ATOM | 5201 | N | PRO | A | 660 | 43.235 | 96.318 | −34.061 | 1.00 | 21.33 | A |
| ATOM | 5202 | CD | PRO | A | 660 | 42.965 | 97.547 | −33.295 | 1.00 | 21.66 | A |
| ATOM | 5203 | CA | PRO | A | 660 | 44.625 | 96.289 | −34.526 | 1.00 | 21.08 | A |
| ATOM | 5204 | CB | PRO | A | 660 | 45.129 | 97.687 | −34.179 | 1.00 | 21.58 | A |
| ATOM | 5205 | CG | PRO | A | 660 | 44.359 | 98.017 | −32.941 | 1.00 | 22.16 | A |
| ATOM | 5206 | C | PRO | A | 660 | 45.405 | 95.190 | −33.812 | 1.00 | 20.87 | A |
| ATOM | 5207 | O | PRO | A | 660 | 45.139 | 94.888 | −32.649 | 1.00 | 20.82 | A |
| ATOM | 5208 | N | LEU | A | 661 | 46.365 | 94.595 | −34.510 | 1.00 | 21.59 | A |
| ATOM | 5209 | CA | LEU | A | 661 | 47.158 | 93.517 | −33.931 | 1.00 | 22.22 | A |
| ATOM | 5210 | CB | LEU | A | 661 | 46.761 | 92.187 | −34.573 | 1.00 | 21.97 | A |
| ATOM | 5211 | CG | LEU | A | 661 | 45.311 | 91.743 | −34.360 | 1.00 | 22.01 | A |
| ATOM | 5212 | CD1 | LEU | A | 661 | 45.039 | 90.474 | −35.154 | 1.00 | 21.83 | A |
| ATOM | 5213 | CD2 | LEU | A | 661 | 45.061 | 91.514 | −32.878 | 1.00 | 21.98 | A |
| ATOM | 5214 | C | LEU | A | 661 | 48.661 | 93.731 | −34.079 | 1.00 | 23.14 | A |
| ATOM | 5215 | O | LEU | A | 661 | 49.430 | 92.771 | −34.137 | 1.00 | 22.28 | A |
| ATOM | 5216 | N | GLY | A | 662 | 49.076 | 94.991 | −34.143 | 1.00 | 24.28 | A |
| ATOM | 5217 | CA | GLY | A | 662 | 50.490 | 95.293 | −34.276 | 1.00 | 25.42 | A |
| ATOM | 5218 | C | GLY | A | 662 | 51.141 | 94.711 | −35.517 | 1.00 | 26.28 | A |
| ATOM | 5219 | O | GLY | A | 662 | 50.702 | 94.961 | −36.639 | 1.00 | 26.88 | A |
| ATOM | 5220 | N | GLN | A | 663 | 52.189 | 93.919 | −35.313 | 1.00 | 26.82 | A |
| ATOM | 5221 | CA | GLN | A | 663 | 52.925 | 93.311 | −36.416 | 1.00 | 27.25 | A |
| ATOM | 5222 | CB | GLN | A | 663 | 54.290 | 92.827 | −35.926 | 1.00 | 29.76 | A |
| ATOM | 5223 | CG | GLN | A | 663 | 55.126 | 93.886 | −35.235 | 1.00 | 32.87 | A |
| ATOM | 5224 | CD | GLN | A | 663 | 56.472 | 93.350 | −34.796 | 1.00 | 34.37 | A |
| ATOM | 5225 | OE1 | GLN | A | 663 | 57.304 | 92.975 | −35.624 | 1.00 | 34.97 | A |
| ATOM | 5226 | NE2 | GLN | A | 663 | 56.692 | 93.302 | −33.486 | 1.00 | 35.06 | A |
| ATOM | 5227 | C | GLN | A | 663 | 52.211 | 92.147 | −37.097 | 1.00 | 25.74 | A |
| ATOM | 5228 | O | GLN | A | 663 | 52.647 | 91.685 | −38.151 | 1.00 | 25.61 | A |
| ATOM | 5229 | N | TYR | A | 664 | 51.122 | 91.669 | −36.502 | 1.00 | 24.78 | A |
| ATOM | 5230 | CA | TYR | A | 664 | 50.390 | 90.545 | −37.077 | 1.00 | 23.54 | A |
| ATOM | 5231 | CB | TYR | A | 664 | 49.099 | 90.298 | −36.296 | 1.00 | 21.17 | A |
| ATOM | 5232 | CG | TYR | A | 664 | 48.511 | 88.923 | −36.523 | 1.00 | 18.46 | A |
| ATOM | 5233 | CD1 | TYR | A | 664 | 49.099 | 87.790 | −35.959 | 1.00 | 17.21 | A |
| ATOM | 5234 | CE1 | TYR | A | 664 | 48.558 | 86.520 | −36.167 | 1.00 | 16.14 | A |
| ATOM | 5235 | CD2 | TYR | A | 664 | 47.369 | 88.754 | −37.304 | 1.00 | 16.89 | A |
| ATOM | 5236 | CE2 | TYR | A | 664 | 46.824 | 87.493 | −37.520 | 1.00 | 16.07 | A |
| ATOM | 5237 | CZ | TYR | A | 664 | 47.421 | 86.381 | −36.949 | 1.00 | 15.48 | A |
| ATOM | 5238 | OH | TYR | A | 664 | 46.871 | 85.138 | −37.157 | 1.00 | 14.24 | A |
| ATOM | 5239 | C | TYR | A | 664 | 50.077 | 90.788 | −38.556 | 1.00 | 23.88 | A |
| ATOM | 5240 | O | TYR | A | 664 | 49.453 | 91.787 | −38.917 | 1.00 | 23.82 | A |
| ATOM | 5241 | N | PRO | A | 665 | 50.511 | 89.865 | −39.430 | 1.00 | 24.84 | A |
| ATOM | 5242 | CD | PRO | A | 665 | 51.277 | 88.674 | −39.021 | 1.00 | 24.79 | A |
| ATOM | 5243 | CA | PRO | A | 665 | 50.341 | 89.881 | −40.887 | 1.00 | 25.24 | A |
| ATOM | 5244 | CB | PRO | A | 665 | 50.859 | 88.507 | −41.304 | 1.00 | 25.87 | A |
| ATOM | 5245 | CG | PRO | A | 665 | 51.930 | 88.253 | −40.314 | 1.00 | 25.62 | A |
| ATOM | 5246 | C | PRO | A | 665 | 48.947 | 90.152 | −41.450 | 1.00 | 25.71 | A |
| ATOM | 5247 | O | PRO | A | 665 | 48.771 | 91.071 | −42.249 | 1.00 | 26.69 | A |
| ATOM | 5248 | N | GLU | A | 666 | 47.960 | 89.357 | −41.048 | 1.00 | 25.11 | A |
| ATOM | 5249 | CA | GLU | A | 666 | 46.609 | 89.525 | −41.575 | 1.00 | 24.71 | A |
| ATOM | 5250 | CB | GLU | A | 666 | 45.952 | 88.160 | −41.804 | 1.00 | 26.60 | A |
| ATOM | 5251 | CG | GLU | A | 666 | 44.712 | 88.238 | −42.688 | 1.00 | 29.67 | A |
| ATOM | 5252 | CD | GLU | A | 666 | 44.229 | 86.881 | −43.159 | 1.00 | 30.79 | A |
| ATOM | 5253 | OE1 | GLU | A | 666 | 45.049 | 86.112 | −43.703 | 1.00 | 32.46 | A |
| ATOM | 5254 | OE2 | GLU | A | 666 | 43.026 | 86.588 | −42.997 | 1.00 | 31.43 | A |
| ATOM | 5255 | C | GLU | A | 666 | 45.689 | 90.392 | −40.728 | 1.00 | 23.13 | A |
| ATOM | 5256 | O | GLU | A | 666 | 45.671 | 90.298 | −39.501 | 1.00 | 22.63 | A |
| ATOM | 5257 | N | ASP | A | 667 | 44.916 | 91.235 | −41.406 | 1.00 | 21.36 | A |
| ATOM | 5258 | CA | ASP | A | 667 | 43.984 | 92.133 | −40.741 | 1.00 | 19.98 | A |
| ATOM | 5259 | CB | ASP | A | 667 | 43.616 | 93.289 | −41.674 | 1.00 | 22.14 | A |
| ATOM | 5260 | CG | ASP | A | 667 | 44.833 | 94.017 | −42.205 | 1.00 | 24.02 | A |
| ATOM | 5261 | OD1 | ASP | A | 667 | 45.630 | 94.522 | −41.387 | 1.00 | 25.50 | A |
| ATOM | 5262 | OD2 | ASP | A | 667 | 44.993 | 94.085 | −43.443 | 1.00 | 26.55 | A |
| ATOM | 5263 | C | ASP | A | 667 | 42.713 | 91.396 | −40.332 | 1.00 | 17.30 | A |
| ATOM | 5264 | O | ASP | A | 667 | 42.220 | 90.536 | −41.060 | 1.00 | 16.65 | A |
| ATOM | 5265 | N | VAL | A | 668 | 42.189 | 91.741 | −39.163 | 1.00 | 16.72 | A |
| ATOM | 5266 | CA | VAL | A | 668 | 40.967 | 91.124 | −38.668 | 1.00 | 15.69 | A |
| ATOM | 5267 | CB | VAL | A | 668 | 40.652 | 91.582 | −37.226 | 1.00 | 15.77 | A |
| ATOM | 5268 | CG1 | VAL | A | 668 | 39.311 | 91.011 | −36.772 | 1.00 | 15.98 | A |
| ATOM | 5269 | CG2 | VAL | A | 668 | 41.761 | 91.127 | −36.286 | 1.00 | 15.95 | A |
| ATOM | 5270 | C | VAL | A | 668 | 39.803 | 91.506 | −39.576 | 1.00 | 15.77 | A |
| ATOM | 5271 | O | VAL | A | 668 | 39.730 | 92.637 | −40.068 | 1.00 | 16.20 | A |
| ATOM | 5272 | N | LYS | A | 669 | 38.903 | 90.554 | −39.800 | 1.00 | 15.11 | A |
| ATOM | 5273 | CA | LYS | A | 669 | 37.729 | 90.772 | −40.640 | 1.00 | 16.26 | A |
| ATOM | 5274 | CB | LYS | A | 669 | 37.589 | 89.626 | −41.643 | 1.00 | 18.36 | A |
| ATOM | 5275 | CG | LYS | A | 669 | 38.834 | 89.429 | −42.500 | 1.00 | 22.33 | A |
| ATOM | 5276 | CD | LYS | A | 669 | 38.865 | 88.069 | −43.178 | 1.00 | 25.02 | A |
| ATOM | 5277 | CE | LYS | A | 669 | 40.204 | 87.847 | −43.872 | 1.00 | 26.17 | A |
| ATOM | 5278 | NZ | LYS | A | 669 | 40.319 | 86.492 | −44.474 | 1.00 | 27.07 | A |
| ATOM | 5279 | C | LYS | A | 669 | 36.501 | 90.841 | −39.738 | 1.00 | 15.79 | A |
| ATOM | 5280 | O | LYS | A | 669 | 36.484 | 90.250 | −38.656 | 1.00 | 15.00 | A |
| ATOM | 5281 | N | PHE | A | 670 | 35.477 | 91.565 | −40.177 | 1.00 | 15.18 | A |
| ATOM | 5282 | CA | PHE | A | 670 | 34.263 | 91.707 | −39.386 | 1.00 | 15.33 | A |
| ATOM | 5283 | CB | PHE | A | 670 | 34.122 | 93.150 | −38.881 | 1.00 | 15.50 | A |
| ATOM | 5284 | CG | PHE | A | 670 | 35.310 | 93.637 | −38.100 | 1.00 | 15.57 | A |
| ATOM | 5285 | CD1 | PHE | A | 670 | 36.455 | 94.082 | −38.755 | 1.00 | 16.30 | A |
| ATOM | 5286 | CD2 | PHE | A | 670 | 35.301 | 93.612 | −36.707 | 1.00 | 16.26 | A |
| ATOM | 5287 | CE1 | PHE | A | 670 | 37.577 | 94.493 | −38.034 | 1.00 | 15.93 | A |
| ATOM | 5288 | CE2 | PHE | A | 670 | 36.417 | 94.020 | −35.978 | 1.00 | 16.44 | A |
| ATOM | 5289 | CZ | PHE | A | 670 | 37.558 | 94.462 | −36.645 | 1.00 | 16.10 | A |
| ATOM | 5290 | C | PHE | A | 670 | 33.019 | 91.312 | −40.168 | 1.00 | 15.07 | A |
| ATOM | 5291 | O | PHE | A | 670 | 33.052 | 91.192 | −41.392 | 1.00 | 15.25 | A |
| ATOM | 5292 | N | GLY | A | 671 | 31.921 | 91.101 | −39.450 | 1.00 | 15.47 | A |
| ATOM | 5293 | CA | GLY | A | 671 | 30.675 | 90.729 | −40.097 | 1.00 | 15.10 | A |
| ATOM | 5294 | C | GLY | A | 671 | 29.551 | 90.550 | −39.097 | 1.00 | 15.27 | A |
| ATOM | 5295 | O | GLY | A | 671 | 29.796 | 90.431 | −37.897 | 1.00 | 14.16 | A |
| ATOM | 5296 | N | ASP | A | 672 | 28.311 | 90.551 | −39.575 | 1.00 | 15.65 | A |
| ATOM | 5297 | CA | ASP | A | 672 | 27.181 | 90.359 | −38.676 | 1.00 | 16.95 | A |
| ATOM | 5298 | CB | ASP | A | 672 | 25.855 | 90.715 | −39.358 | 1.00 | 18.42 | A |
| ATOM | 5299 | CG | ASP | A | 672 | 25.741 | 92.188 | −39.695 | 1.00 | 20.16 | A |
| ATOM | 5300 | OD1 | ASP | A | 672 | 26.285 | 93.021 | −38.942 | 1.00 | 21.62 | A |
| ATOM | 5301 | OD2 | ASP | A | 672 | 25.085 | 92.510 | −40.707 | 1.00 | 22.09 | A |
| ATOM | 5302 | C | ASP | A | 672 | 27.137 | 88.892 | −38.271 | 1.00 | 17.16 | A |
| ATOM | 5303 | O | ASP | A | 672 | 27.617 | 88.027 | −39.002 | 1.00 | 16.41 | A |
| ATOM | 5304 | N | PRO | A | 673 | 26.565 | 88.594 | −37.094 | 1.00 | 17.20 | A |
| ATOM | 5305 | CD | PRO | A | 673 | 25.975 | 89.505 | −36.095 | 1.00 | 17.66 | A |
| ATOM | 5306 | CA | PRO | A | 673 | 26.483 | 87.202 | −36.647 | 1.00 | 17.71 | A |
| ATOM | 5307 | CB | PRO | A | 673 | 25.575 | 87.291 | −35.428 | 1.00 | 17.79 | A |
| ATOM | 5308 | CG | PRO | A | 673 | 25.920 | 88.630 | −34.858 | 1.00 | 18.25 | A |
| ATOM | 5309 | C | PRO | A | 673 | 25.883 | 86.332 | −37.750 | 1.00 | 17.63 | A |
| ATOM | 5310 | O | PRO | A | 673 | 24.986 | 86.766 | −38.479 | 1.00 | 17.66 | A |
| ATOM | 5311 | N | ARG | A | 674 | 26.393 | 85.114 | −37.881 | 1.00 | 17.42 | A |
| ATOM | 5312 | CA | ARG | A | 674 | 25.901 | 84.183 | −38.887 | 1.00 | 17.69 | A |
| ATOM | 5313 | CB | ARG | A | 674 | 26.367 | 84.596 | −40.289 | 1.00 | 18.49 | A |
| ATOM | 5314 | CG | ARG | A | 674 | 27.866 | 84.469 | −40.540 | 1.00 | 19.76 | A |
| ATOM | 5315 | CD | ARG | A | 674 | 28.155 | 84.525 | −42.038 | 1.00 | 21.74 | A |
| ATOM | 5316 | NE | ARG | A | 674 | 29.545 | 84.220 | −42.371 | 1.00 | 23.11 | A |
| ATOM | 5317 | CZ | ARG | A | 674 | 30.535 | 85.107 | −42.374 | 1.00 | 23.99 | A |
| ATOM | 5318 | NH1 | ARG | A | 674 | 30.301 | 86.375 | −42.059 | 1.00 | 24.74 | A |
| ATOM | 5319 | NH2 | ARG | A | 674 | 31.763 | 84.727 | −42.703 | 1.00 | 24.81 | A |
| ATOM | 5320 | C | ARG | A | 674 | 26.422 | 82.792 | −38.575 | 1.00 | 17.54 | A |
| ATOM | 5321 | O | ARG | A | 674 | 27.386 | 82.639 | −37.825 | 1.00 | 17.04 | A |
| ATOM | 5322 | N | GLU | A | 675 | 25.780 | 81.779 | −39.144 | 1.00 | 18.31 | A |
| ATOM | 5323 | CA | GLU | A | 675 | 26.218 | 80.413 | −38.919 | 1.00 | 19.05 | A |
| ATOM | 5324 | CB | GLU | A | 675 | 25.157 | 79.420 | −39.396 | 1.00 | 21.17 | A |
| ATOM | 5325 | CG | GLU | A | 675 | 23.805 | 79.621 | −38.744 | 1.00 | 23.24 | A |
| ATOM | 5326 | CD | GLU | A | 675 | 22.959 | 78.368 | −38.757 | 1.00 | 25.19 | A |
| ATOM | 5327 | OE1 | GLU | A | 675 | 22.873 | 77.713 | −39.816 | 1.00 | 26.11 | A |
| ATOM | 5328 | OE2 | GLU | A | 675 | 22.373 | 73.042 | −37.704 | 1.00 | 26.65 | A |
| ATOM | 5329 | C | GLU | A | 675 | 27.519 | 80.198 | −39.679 | 1.00 | 19.07 | A |
| ATOM | 5330 | O | GLU | A | 675 | 27.723 | 80.767 | −40.754 | 1.00 | 19.20 | A |
| ATOM | 5331 | N | ILE | A | 676 | 28.403 | 79.384 | −39.115 | 1.00 | 18.72 | A |
| ATOM | 5332 | CA | ILE | A | 676 | 29.686 | 79.113 | −39.745 | 1.00 | 19.45 | A |
| ATOM | 5333 | CB | ILE | A | 676 | 30.816 | 79.902 | −39.066 | 1.00 | 21.20 | A |
| ATOM | 5334 | CG2 | ILE | A | 676 | 30.544 | 81.398 | −39.162 | 1.00 | 23.12 | A |
| ATOM | 5335 | CG1 | ILE | A | 676 | 30.934 | 79.469 | −37.605 | 1.00 | 22.04 | A |
| ATOM | 5336 | CD1 | ILE | A | 676 | 32.131 | 80.041 | −36.886 | 1.00 | 23.61 | A |
| ATOM | 5337 | C | ILE | A | 676 | 30.042 | 77.637 | −39.665 | 1.00 | 18.82 | A |
| ATOM | 5338 | O | ILE | A | 676 | 29.551 | 76.914 | −38.796 | 1.00 | 18.47 | A |
| ATOM | 5339 | N | SER | A | 677 | 30.905 | 77.207 | −40.577 | 1.00 | 18.44 | A |
| ATOM | 5340 | CA | SER | A | 677 | 31.370 | 75.828 | −40.630 | 1.00 | 18.86 | A |
| ATOM | 5341 | CB | SER | A | 677 | 30.769 | 75.113 | −41.842 | 1.00 | 20.56 | A |
| ATOM | 5342 | OG | SER | A | 677 | 31.231 | 73.777 | −41.925 | 1.00 | 24.04 | A |
| ATOM | 5343 | C | SER | A | 677 | 32.889 | 75.850 | −40.743 | 1.00 | 18.55 | A |
| ATOM | 5344 | O | SER | A | 677 | 33.451 | 76.667 | −41.474 | 1.00 | 18.59 | A |
| ATOM | 5345 | N | LEU | A | 678 | 33.554 | 74.956 | −40.019 | 1.00 | 17.21 | A |
| ATOM | 5346 | CA | LEU | A | 678 | 35.009 | 74.895 | −40.039 | 1.00 | 17.53 | A |
| ATOM | 5347 | CB | LEU | A | 678 | 35.587 | 75.588 | −38.802 | 1.00 | 18.74 | A |
| ATOM | 5348 | CG | LEU | A | 678 | 35.405 | 77.100 | −38.669 | 1.00 | 19.72 | A |
| ATOM | 5349 | CD1 | LEU | A | 678 | 35.871 | 77.548 | −37.292 | 1.00 | 20.83 | A |
| ATOM | 5350 | CD2 | LEU | A | 678 | 36.192 | 77.808 | −39.759 | 1.00 | 21.63 | A |
| ATOM | 5351 | C | LEU | A | 678 | 35.522 | 73.465 | −40.075 | 1.00 | 17.34 | A |
| ATOM | 5352 | O | LEU | A | 678 | 34.862 | 72.544 | −39.591 | 1.00 | 16.69 | A |
| ATOM | 5353 | N | ARG | A | 679 | 36.708 | 73.295 | −40.650 | 1.00 | 17.12 | A |
| ATOM | 5354 | CA | ARG | A | 679 | 37.344 | 71.990 | −40.733 | 1.00 | 18.44 | A |
| ATOM | 5355 | CB | ARG | A | 679 | 36.912 | 71.248 | −42.001 | 1.00 | 20.29 | A |
| ATOM | 5356 | CG | ARG | A | 679 | 37.461 | 69.829 | −42.063 | 1.00 | 23.21 | A |
| ATOM | 5357 | CD | ARG | A | 679 | 37.211 | 69.164 | −43.403 | 1.00 | 25.74 | A |
| ATOM | 5358 | NE | ARG | A | 679 | 37.627 | 67.765 | −43.377 | 1.00 | 28.32 | A |
| ATOM | 5359 | CZ | ARG | A | 679 | 37.683 | 66.979 | −44.446 | 1.00 | 29.41 | A |
| ATOM | 5360 | NH1 | ARG | A | 679 | 37.352 | 67.455 | −45.639 | 1.00 | 30.45 | A |
| ATOM | 5361 | NH2 | ARG | A | 679 | 38.068 | 65.716 | −44.322 | 1.00 | 30.28 | A |
| ATOM | 5362 | C | ARG | A | 679 | 38.860 | 72.146 | −40.741 | 1.00 | 18.22 | A |
| ATOM | 5363 | O | ARG | A | 679 | 39.412 | 72.892 | −41.548 | 1.00 | 17.86 | A |
| ATOM | 5364 | N | VAL | A | 680 | 39.528 | 71.447 | −39.831 | 1.00 | 17.40 | A |
| ATOM | 5365 | CA | VAL | A | 680 | 40.980 | 71.489 | −39.755 | 1.00 | 17.92 | A |
| ATOM | 5366 | CB | VAL | A | 680 | 41.460 | 71.762 | −38.312 | 1.00 | 17.20 | A |
| ATOM | 5367 | CG1 | VAL | A | 680 | 42.967 | 71.619 | −38.228 | 1.00 | 17.12 | A |
| ATOM | 5368 | CG2 | VAL | A | 680 | 41.041 | 73.163 | −37.888 | 1.00 | 15.30 | A |
| ATOM | 5369 | C | VAL | A | 680 | 41.514 | 70.141 | −40.222 | 1.00 | 19.22 | A |
| ATOM | 5370 | O | VAL | A | 680 | 41.033 | 69.095 | −39.788 | 1.00 | 19.39 | A |
| ATOM | 5371 | N | GLY | A | 681 | 42.500 | 70.173 | −41.115 | 1.00 | 20.72 | A |
| ATOM | 5372 | CA | GLY | A | 681 | 43.071 | 68.943 | −41.634 | 1.00 | 22.99 | A |
| ATOM | 5373 | C | GLY | A | 681 | 42.000 | 68.071 | −42.262 | 1.00 | 24.42 | A |
| ATOM | 5374 | O | GLY | A | 681 | 41.101 | 68.571 | −42.937 | 1.00 | 24.23 | A |
| ATOM | 5375 | N | ASN | A | 682 | 42.098 | 66.763 | −42.046 | 1.00 | 26.46 | A |
| ATOM | 5376 | CA | ASN | A | 682 | 41.116 | 65.828 | −42.580 | 1.00 | 28.26 | A |
| ATOM | 5377 | CB | ASN | A | 682 | 41.809 | 64.595 | −43.165 | 1.00 | 30.25 | A |
| ATOM | 5378 | CG | ASN | A | 682 | 42.484 | 64.879 | −44.493 | 1.00 | 32.05 | A |
| ATOM | 5379 | OD1 | ASN | A | 682 | 43.151 | 64.013 | −45.058 | 1.00 | 34.30 | A |
| ATOM | 5380 | ND2 | ASN | A | 682 | 42.308 | 66.093 | −45.003 | 1.00 | 33.43 | A |
| ATOM | 5381 | C | ASN | A | 682 | 40.174 | 65.411 | −41.461 | 1.00 | 27.97 | A |
| ATOM | 5382 | O | ASN | A | 682 | 39.468 | 64.408 | −41.564 | 1.00 | 28.97 | A |
| ATOM | 5383 | N | GLY | A | 683 | 40.172 | 66.198 | −40.389 | 1.00 | 26.89 | A |
| ATOM | 5384 | CA | GLY | A | 683 | 39.320 | 65.909 | −39.254 | 1.00 | 24.95 | A |
| ATOM | 5385 | C | GLY | A | 683 | 37.859 | 66.178 | −39.547 | 1.00 | 23.15 | A |
| ATOM | 5386 | O | GLY | A | 683 | 37.475 | 66.319 | −40.708 | 1.00 | 23.65 | A |
| ATOM | 5387 | N | PRO | A | 684 | 37.015 | 66.260 | −38.508 | 1.00 | 21.20 | A |
| ATOM | 5388 | CD | PRO | A | 684 | 37.343 | 66.081 | −37.081 | 1.00 | 21.32 | A |
| ATOM | 5389 | CA | PRO | A | 684 | 35.584 | 66.515 | −38.685 | 1.00 | 19.81 | A |
| ATOM | 5390 | CB | PRO | A | 684 | 35.007 | 66.164 | −37.319 | 1.00 | 20.16 | A |
| ATOM | 5391 | CG | PRO | A | 684 | 36.099 | 66.605 | −36.391 | 1.00 | 20.87 | A |
| ATOM | 5392 | C | PRO | A | 684 | 35.272 | 67.951 | −39.088 | 1.00 | 18.75 | A |
| ATOM | 5393 | O | PRO | A | 684 | 36.086 | 68.853 | −38.905 | 1.00 | 18.07 | A |
| ATOM | 5394 | N | THR | A | 685 | 34.087 | 68.147 | −39.651 | 1.00 | 17.24 | A |
| ATOM | 5395 | CA | THR | A | 685 | 33.634 | 69.470 | −40.051 | 1.00 | 16.41 | A |
| ATOM | 5396 | CB | THR | A | 685 | 33.020 | 69.452 | −41.462 | 1.00 | 16.39 | A |
| ATOM | 5397 | OG1 | THR | A | 685 | 34.041 | 69.153 | −42.420 | 1.00 | 17.54 | A |
| ATOM | 5398 | CG2 | THR | A | 685 | 32.400 | 70.804 | −41.791 | 1.00 | 16.76 | A |
| ATOM | 5399 | C | THR | A | 685 | 32.573 | 69.863 | −39.034 | 1.00 | 15.52 | A |
| ATOM | 5400 | O | THR | A | 685 | 31.573 | 69.166 | −38.872 | 1.00 | 16.19 | A |
| ATOM | 5401 | N | LEU | A | 686 | 32.801 | 70.971 | −38.338 | 1.00 | 14.31 | A |
| ATOM | 5402 | CA | LEU | A | 686 | 31.865 | 71.429 | −37.321 | 1.00 | 14.02 | A |
| ATOM | 5403 | CB | LEU | A | 686 | 32.612 | 71.775 | −36.027 | 1.00 | 14.50 | A |
| ATOM | 5404 | CG | LEU | A | 686 | 33.527 | 70.713 | −35.415 | 1.00 | 16.33 | A |
| ATOM | 5405 | CD1 | LEU | A | 686 | 34.004 | 71.202 | −34.058 | 1.00 | 15.65 | A |
| ATOM | 5406 | CD2 | LEU | A | 686 | 32.796 | 69.394 | −35.273 | 1.00 | 16.36 | A |
| ATOM | 5407 | C | LEU | A | 686 | 31.073 | 72.646 | −37.770 | 1.00 | 13.55 | A |
| ATOM | 5408 | O | LEU | A | 686 | 31.625 | 73.570 | −38.369 | 1.00 | 13.84 | A |
| ATOM | 5409 | N | ALA | A | 687 | 29.778 | 72.638 | −37.475 | 1.00 | 12.69 | A |
| ATOM | 5410 | CA | ALA | A | 687 | 28.898 | 73.746 | −37.822 | 1.00 | 12.84 | A |
| ATOM | 5411 | CB | ALA | A | 687 | 27.683 | 73.236 | −38.592 | 1.00 | 12.65 | A |
| ATOM | 5412 | C | ALA | A | 687 | 28.454 | 74.430 | −36.534 | 1.00 | 12.45 | A |
| ATOM | 5413 | O | ALA | A | 687 | 28.133 | 73.763 | −35.544 | 1.00 | 12.43 | A |
| ATOM | 5414 | N | PHE | A | 688 | 28.436 | 75.761 | −36.549 | 1.00 | 12.17 | A |
| ATOM | 5415 | CA | PHE | A | 688 | 28.042 | 76.535 | −35.380 | 1.00 | 11.59 | A |
| ATOM | 5416 | CB | PHE | A | 688 | 29.215 | 77.384 | −34.884 | 1.00 | 11.95 | A |
| ATOM | 5417 | CG | PHE | A | 688 | 30.453 | 76.596 | −34.591 | 1.00 | 11.60 | A |
| ATOM | 5418 | CD1 | PHE | A | 688 | 31.252 | 76.122 | −35.623 | 1.00 | 11.51 | A |
| ATOM | 5419 | CD2 | PHE | A | 688 | 30.810 | 76.310 | −33.278 | 1.00 | 11.55 | A |
| ATOM | 5420 | CE1 | PHE | A | 688 | 32.394 | 75.369 | −35.355 | 1.00 | 11.61 | A |
| ATOM | 5421 | CE2 | PHE | A | 688 | 31.950 | 75.558 | −32.999 | 1.00 | 11.40 | A |
| ATOM | 5422 | CZ | PHE | A | 688 | 32.743 | 75.087 | −34.039 | 1.00 | 11.47 | A |
| ATOM | 5423 | C | PHE | A | 688 | 26.876 | 77.460 | −35.677 | 1.00 | 12.06 | A |
| ATOM | 5424 | O | PHE | A | 688 | 26.697 | 77.900 | −36.811 | 1.00 | 13.38 | A |
| ATOM | 5425 | N | SER | A | 689 | 26.091 | 77.756 | −34.649 | 1.00 | 12.46 | A |
| ATOM | 5426 | CA | SER | A | 689 | 24.955 | 78.662 | −34.780 | 1.00 | 13.44 | A |
| ATOM | 5427 | CB | SER | A | 689 | 24.022 | 78.518 | −33.579 | 1.00 | 13.79 | A |
| ATOM | 5428 | OG | SER | A | 689 | 24.633 | 79.032 | −32.407 | 1.00 | 14.51 | A |
| ATOM | 5429 | C | SER | A | 689 | 25.518 | 80.081 | −34.806 | 1.00 | 14.49 | A |
| ATOM | 5430 | O | SER | A | 689 | 26.713 | 80.282 | −34.590 | 1.00 | 14.70 | A |
| ATOM | 5431 | N | GLU | A | 690 | 24.665 | 81.066 | −35.066 | 1.00 | 15.35 | A |
| ATOM | 5432 | CA | GLU | A | 690 | 25.133 | 82.447 | −35.096 | 1.00 | 16.16 | A |
| ATOM | 5433 | CB | GLU | A | 690 | 24.073 | 83.367 | −35.713 | 1.00 | 18.48 | A |
| ATOM | 5434 | CG | GLU | A | 690 | 22.813 | 83.539 | −34.896 | 1.00 | 21.37 | A |
| ATOM | 5435 | CD | GLU | A | 690 | 21.855 | 84.530 | −35.532 | 1.00 | 23.24 | A |
| ATOM | 5436 | OE1 | GLU | A | 690 | 21.335 | 84.233 | −36.628 | 1.00 | 25.21 | A |
| ATOM | 5437 | OE2 | GLU | A | 690 | 21.632 | 85.608 | −34.941 | 1.00 | 25.58 | A |
| ATOM | 5438 | C | GLU | A | 690 | 25.493 | 82.926 | −33.691 | 1.00 | 16.10 | A |
| ATOM | 5439 | O | GLU | A | 690 | 25.997 | 84.035 | −33.513 | 1.00 | 15.36 | A |
| ATOM | 5440 | N | GLN | A | 691 | 25.232 | 82.084 | −32.695 | 1.00 | 15.90 | A |
| ATOM | 5441 | CA | GLN | A | 691 | 25.554 | 82.413 | −31.310 | 1.00 | 16.38 | A |
| ATOM | 5442 | CB | GLN | A | 691 | 24.451 | 81.913 | −30.378 | 1.00 | 18.37 | A |
| ATOM | 5443 | CG | GLN | A | 691 | 23.129 | 82.632 | −30.574 | 1.00 | 21.65 | A |
| ATOM | 5444 | CD | GLN | A | 691 | 21.940 | 81.747 | −30.275 | 1.00 | 24.27 | A |
| ATOM | 5445 | OE1 | GLN | A | 691 | 21.755 | 81.289 | −29.148 | 1.00 | 25.45 | A |
| ATOM | 5446 | NE2 | GLN | A | 691 | 21.125 | 81.495 | −31.293 | 1.00 | 25.38 | A |
| ATOM | 5447 | C | GLN | A | 691 | 26.892 | 81.783 | −30.928 | 1.00 | 14.99 | A |
| ATOM | 5448 | O | GLN | A | 691 | 27.304 | 81.830 | −29.769 | 1.00 | 16.19 | A |
| ATOM | 5449 | N | GLY | A | 692 | 27.557 | 81.187 | −31.915 | 1.00 | 13.74 | A |
| ATOM | 5450 | CA | GLY | A | 692 | 28.856 | 80.576 | −31.689 | 1.00 | 13.44 | A |
| ATOM | 5451 | C | GLY | A | 692 | 28.843 | 79.231 | −30.992 | 1.00 | 12.84 | A |
| ATOM | 5452 | O | GLY | A | 692 | 29.870 | 78.788 | −30.482 | 1.00 | 12.87 | A |
| ATOM | 5453 | N | LEU | A | 693 | 27.690 | 78.572 | −30.982 | 1.00 | 12.59 | A |
| ATOM | 5454 | CA | LEU | A | 693 | 27.563 | 77.270 | −30.333 | 1.00 | 12.95 | A |
| ATOM | 5455 | CB | LEU | A | 693 | 26.332 | 77.257 | −29.427 | 1.00 | 14.47 | A |
| ATOM | 5456 | CG | LEU | A | 693 | 26.373 | 78.185 | −28.211 | 1.00 | 15.50 | A |
| ATOM | 5457 | CD1 | LEU | A | 693 | 24.963 | 78.374 | −27.671 | 1.00 | 16.99 | A |
| ATOM | 5458 | CD2 | LEU | A | 693 | 27.294 | 77.606 | −27.143 | 1.00 | 16.14 | A |
| ATOM | 5459 | C | LEU | A | 693 | 27.460 | 76.141 | −31.347 | 1.00 | 12.58 | A |
| ATOM | 5460 | O | LEU | A | 693 | 26.797 | 76.269 | −32.377 | 1.00 | 13.69 | A |
| ATOM | 5461 | N | LEU | A | 694 | 28.121 | 75.030 | −31.045 | 1.00 | 12.10 | A |
| ATOM | 5462 | CA | LEU | A | 694 | 28.107 | 73.868 | −31.921 | 1.00 | 12.19 | A |
| ATOM | 5463 | CB | LEU | A | 694 | 28.833 | 72.702 | −31.246 | 1.00 | 11.89 | A |
| ATOM | 5464 | CG | LEU | A | 694 | 29.022 | 71.436 | −32.082 | 1.00 | 12.29 | A |
| ATOM | 5465 | CD1 | LEU | A | 694 | 29.975 | 71.722 | −33.223 | 1.00 | 12.88 | A |
| ATOM | 5466 | CD2 | LEU | A | 694 | 29.583 | 70.320 | −31.207 | 1.00 | 12.25 | A |
| ATOM | 5467 | C | LEU | A | 694 | 26.681 | 73.445 | −32.261 | 1.00 | 12.42 | A |
| ATOM | 5468 | O | LEU | A | 694 | 25.807 | 73.430 | −31.397 | 1.00 | 12.36 | A |
| ATOM | 5469 | N | LYS | A | 695 | 26.460 | 73.101 | −33.527 | 1.00 | 13.99 | A |
| ATOM | 5470 | CA | LYS | A | 695 | 25.146 | 72.662 | −33.989 | 1.00 | 15.49 | A |
| ATOM | 5471 | CB | LYS | A | 695 | 24.586 | 73.660 | −35.011 | 1.00 | 18.76 | A |
| ATOM | 5472 | CG | LYS | A | 695 | 23.323 | 73.196 | −35.720 | 1.00 | 23.70 | A |
| ATOM | 5473 | CD | LYS | A | 695 | 22.622 | 74.345 | −36.438 | 1.00 | 25.84 | A |
| ATOM | 5474 | CE | LYS | A | 695 | 22.035 | 75.337 | −35.442 | 1.00 | 27.62 | A |
| ATOM | 5475 | NZ | LYS | A | 695 | 21.248 | 76.413 | −36.105 | 1.00 | 29.16 | A |
| ATOM | 5476 | C | LYS | A | 695 | 25.195 | 71.266 | −34.601 | 1.00 | 14.81 | A |
| ATOM | 5477 | O | LYS | A | 695 | 24.228 | 70.509 | −34.502 | 1.00 | 15.17 | A |
| ATOM | 5478 | N | SER | A | 696 | 26.316 | 70.923 | −35.228 | 1.00 | 14.07 | A |
| ATOM | 5479 | CA | SER | A | 696 | 26.461 | 69.612 | −35.854 | 1.00 | 14.25 | A |
| ATOM | 5480 | CB | SER | A | 696 | 25.703 | 69.574 | −37.189 | 1.00 | 15.32 | A |
| ATOM | 5481 | OG | SER | A | 696 | 26.328 | 70.388 | −38.168 | 1.00 | 16.47 | A |
| ATOM | 5482 | C | SER | A | 696 | 27.917 | 69.232 | −36.092 | 1.00 | 14.33 | A |
| ATOM | 5483 | O | SER | A | 696 | 28.802 | 70.093 | −36.124 | 1.00 | 13.52 | A |
| ATOM | 5484 | N | ILE | A | 697 | 28.151 | 67.932 | −36.256 | 1.00 | 13.96 | A |
| ATOM | 5485 | CA | ILE | A | 697 | 29.483 | 67.393 | −36.508 | 1.00 | 14.22 | A |
| ATOM | 5486 | CB | ILE | A | 697 | 30.032 | 66.600 | −35.293 | 1.00 | 13.78 | A |
| ATOM | 5487 | CG2 | ILE | A | 697 | 31.390 | 66.011 | −35.637 | 1.00 | 13.83 | A |
| ATOM | 5488 | CG1 | ILE | A | 697 | 30.148 | 67.504 | −34.066 | 1.00 | 12.86 | A |
| ATOM | 5489 | CD1 | ILE | A | 697 | 30.618 | 66.766 | −32.813 | 1.00 | 12.78 | A |
| ATOM | 5490 | C | ILE | A | 697 | 29.424 | 66.422 | −37.683 | 1.00 | 15.65 | A |
| ATOM | 5491 | O | ILE | A | 697 | 28.589 | 65.518 | −37.700 | 1.00 | 16.00 | A |
| ATOM | 5492 | N | GLN | A | 698 | 30.306 | 66.614 | −38.658 | 1.00 | 15.69 | A |
| ATOM | 5493 | CA | GLN | A | 698 | 30.370 | 65.729 | −39.816 | 1.00 | 17.15 | A |
| ATOM | 5494 | CB | GLN | A | 698 | 30.268 | 66.519 | −41.123 | 1.00 | 18.03 | A |
| ATOM | 5495 | CG | GLN | A | 698 | 29.969 | 65.629 | −42.324 | 1.00 | 19.29 | A |
| ATOM | 5496 | CD | GLN | A | 698 | 30.226 | 66.307 | −43.654 | 1.00 | 20.84 | A |
| ATOM | 5497 | OE1 | GLN | A | 698 | 29.680 | 65.904 | −44.683 | 1.00 | 22.50 | A |
| ATOM | 5498 | NE2 | GLN | A | 698 | 31.073 | 67.327 | −43.647 | 1.00 | 20.89 | A |
| ATOM | 5499 | C | GLN | A | 698 | 31.726 | 65.043 | −39.743 | 1.00 | 17.98 | A |
| ATOM | 5500 | O | GLN | A | 698 | 32.758 | 65.675 | −39.957 | 1.00 | 17.04 | A |
| ATOM | 5501 | N | LEU | A | 699 | 31.725 | 63.748 | −39.440 | 1.00 | 19.25 | A |
| ATOM | 5502 | CA | LEU | A | 699 | 32.970 | 63.000 | −39.306 | 1.00 | 21.55 | A |
| ATOM | 5503 | CB | LEU | A | 699 | 32.673 | 61.570 | −38.841 | 1.00 | 21.34 | A |
| ATOM | 5504 | CG | LEU | A | 699 | 32.002 | 61.458 | −37.469 | 1.00 | 21.19 | A |
| ATOM | 5505 | CD1 | LEU | A | 699 | 31.836 | 59.991 | −37.098 | 1.00 | 20.78 | A |
| ATOM | 5506 | CD2 | LEU | A | 699 | 32.844 | 62.180 | −36.426 | 1.00 | 20.96 | A |
| ATOM | 5507 | C | LEU | A | 699 | 33.829 | 62.966 | −40.564 | 1.00 | 23.75 | A |
| ATOM | 5508 | O | LEU | A | 699 | 35.041 | 63.176 | −40.498 | 1.00 | 23.54 | A |
| ATOM | 5509 | N | THR | A | 700 | 33.205 | 62.700 | −41.706 | 1.00 | 26.14 | A |
| ATOM | 5510 | CA | THR | A | 700 | 33.930 | 62.644 | −42.969 | 1.00 | 29.36 | A |
| ATOM | 5511 | CB | THR | A | 700 | 34.070 | 61.192 | −43.472 | 1.00 | 29.32 | A |
| ATOM | 5512 | OG1 | THR | A | 700 | 32.770 | 60.641 | −43.717 | 1.00 | 29.71 | A |
| ATOM | 5513 | CG2 | THR | A | 700 | 34.790 | 60.339 | −42.439 | 1.00 | 29.80 | A |
| ATOM | 5514 | C | THR | A | 700 | 33.214 | 63.465 | −44.034 | 1.00 | 31.01 | A |
| ATOM | 5515 | O | THR | A | 700 | 32.058 | 63.849 | −43.863 | 1.00 | 31.06 | A |
| ATOM | 5516 | N | GLN | A | 701 | 33.908 | 63.734 | −45.135 | 1.00 | 33.87 | A |
| ATOM | 5517 | CA | GLN | A | 701 | 33.334 | 64.515 | −46.223 | 1.00 | 36.58 | A |
| ATOM | 5518 | CB | GLN | A | 701 | 34.371 | 64.714 | −47.331 | 1.00 | 38.30 | A |
| ATOM | 5519 | CG | GLN | A | 701 | 35.669 | 65.340 | −46.849 | 1.00 | 40.75 | A |
| ATOM | 5520 | CD | GLN | A | 701 | 36.609 | 65.693 | −47.985 | 1.00 | 42.07 | A |
| ATOM | 5521 | OE1 | GLN | A | 701 | 36.275 | 66.501 | −48.852 | 1.00 | 42.75 | A |
| ATOM | 5522 | NE2 | GLN | A | 701 | 37.793 | 65.089 | −47.985 | 1.00 | 42.60 | A |
| ATOM | 5523 | C | GLN | A | 701 | 32.096 | 63.830 | −46.793 | 1.00 | 37.22 | A |
| ATOM | 5524 | O | GLN | A | 701 | 31.206 | 64.487 | −47.333 | 1.00 | 37.80 | A |
| ATOM | 5525 | N | ASP | A | 702 | 32.045 | 62.509 | −46.659 | 1.00 | 37.89 | A |
| ATOM | 5526 | CA | ASP | A | 702 | 30.924 | 61.725 | −47.164 | 1.00 | 38.35 | A |
| ATOM | 5527 | CB | ASP | A | 702 | 31.383 | 60.299 | −47.477 | 1.00 | 39.66 | A |
| ATOM | 5528 | CG | ASP | A | 702 | 32.727 | 60.259 | −48.174 | 1.00 | 40.51 | A |
| ATOM | 5529 | OD1 | ASP | A | 702 | 32.854 | 60.852 | −49.267 | 1.00 | 40.84 | A |
| ATOM | 5530 | OD2 | ASP | A | 702 | 33.659 | 59.633 | −47.624 | 1.00 | 41.15 | A |
| ATOM | 5531 | C | ASP | A | 702 | 29.784 | 61.668 | −46.152 | 1.00 | 37.81 | A |
| ATOM | 5532 | O | ASP | A | 702 | 28.623 | 61.901 | −46.492 | 1.00 | 38.20 | A |
| ATOM | 5533 | N | SER | A | 703 | 30.130 | 61.355 | −44.907 | 1.00 | 36.53 | A |
| ATOM | 5534 | CA | SER | A | 703 | 29.155 | 61.244 | −43.829 | 1.00 | 34.85 | A |
| ATOM | 5535 | CB | SER | A | 703 | 29.877 | 61.019 | −42.498 | 1.00 | 35.05 | A |
| ATOM | 5536 | OG | SER | A | 703 | 30.729 | 62.108 | −42.191 | 1.00 | 34.27 | A |
| ATOM | 5537 | C | SER | A | 703 | 28.242 | 62.462 | −43.715 | 1.00 | 33.65 | A |
| ATOM | 5538 | O | SER | A | 703 | 28.536 | 63.527 | −44.256 | 1.00 | 33.57 | A |
| ATOM | 5539 | N | PRO | A | 704 | 27.109 | 62.310 | −43.008 | 1.00 | 32.60 | A |
| ATOM | 5540 | CD | PRO | A | 704 | 26.549 | 61.025 | −42.547 | 1.00 | 32.80 | A |
| ATOM | 5541 | CA | PRO | A | 704 | 26.141 | 63.393 | −42.816 | 1.00 | 31.41 | A |
| ATOM | 5542 | CB | PRO | A | 704 | 24.836 | 62.639 | −42.613 | 1.00 | 32.30 | A |
| ATOM | 5543 | CG | PRO | A | 704 | 25.286 | 61.454 | −41.823 | 1.00 | 32.47 | A |
| ATOM | 5544 | C | PRO | A | 704 | 26.468 | 64.304 | −41.632 | 1.00 | 30.08 | A |
| ATOM | 5545 | O | PRO | A | 704 | 27.347 | 64.003 | −40.824 | 1.00 | 29.54 | A |
| ATOM | 5546 | N | HIS | A | 705 | 25.750 | 65.420 | −41.543 | 1.00 | 28.15 | A |
| ATOM | 5547 | CA | HIS | A | 705 | 25.939 | 66.380 | −40.461 | 1.00 | 26.08 | A |
| ATOM | 5548 | CB | HIS | A | 705 | 25.513 | 67.777 | −40.917 | 1.00 | 27.16 | A |
| ATOM | 5549 | CG | HIS | A | 705 | 26.327 | 68.315 | −42.051 | 1.00 | 28.61 | A |
| ATOM | 5550 | CD2 | HIS | A | 705 | 26.004 | 68.560 | −43.343 | 1.00 | 29.18 | A |
| ATOM | 5551 | ND1 | HIS | A | 705 | 27.659 | 68.644 | −41.921 | 1.00 | 28.94 | A |
| ATOM | 5552 | CE1 | HIS | A | 705 | 28.122 | 69.067 | −43.084 | 1.00 | 29.49 | A |
| ATOM | 5553 | NE2 | HIS | A | 705 | 27.138 | 69.025 | −43.964 | 1.00 | 29.86 | A |
| ATOM | 5554 | C | HIS | A | 705 | 25.107 | 65.948 | −39.259 | 1.00 | 24.00 | A |
| ATOM | 5555 | O | HIS | A | 705 | 23.914 | 66.235 | −39.178 | 1.00 | 23.98 | A |
| ATOM | 5556 | N | VAL | A | 706 | 25.747 | 65.256 | −38.322 | 1.00 | 21.51 | A |
| ATOM | 5557 | CA | VAL | A | 706 | 25.063 | 64.772 | −37.132 | 1.00 | 18.99 | A |
| ATOM | 5558 | CB | VAL | A | 706 | 25.909 | 63.709 | −36.407 | 1.00 | 18.10 | A |
| ATOM | 5559 | CG1 | VAL | A | 706 | 25.145 | 63.165 | −35.214 | 1.00 | 17.35 | A |
| ATOM | 5560 | CG2 | VAL | A | 706 | 26.273 | 62.593 | −37.370 | 1.00 | 18.04 | A |
| ATOM | 5561 | C | VAL | A | 706 | 24.745 | 65.886 | −36.141 | 1.00 | 18.65 | A |
| ATOM | 5562 | O | VAL | A | 706 | 25.638 | 66.602 | −35.695 | 1.00 | 17.89 | A |
| ATOM | 5563 | N | PRO | A | 707 | 23.462 | 66.046 | −35.787 | 1.00 | 17.80 | A |
| ATOM | 5564 | CD | PRO | A | 707 | 22.283 | 65.387 | −36.380 | 1.00 | 18.52 | A |
| ATOM | 5565 | CA | PRO | A | 707 | 23.053 | 67.084 | −34.838 | 1.00 | 17.47 | A |
| ATOM | 5566 | CB | PRO | A | 707 | 21.532 | 66.942 | −34.802 | 1.00 | 18.04 | A |
| ATOM | 5567 | CG | PRO | A | 707 | 21.209 | 66.424 | −36.175 | 1.00 | 18.64 | A |
| ATOM | 5568 | C | PRO | A | 707 | 23.678 | 66.892 | −33.455 | 1.00 | 16.45 | A |
| ATOM | 5569 | O | PRO | A | 707 | 23.489 | 65.860 | −32.809 | 1.00 | 16.71 | A |
| ATOM | 5570 | N | VAL | A | 708 | 24.441 | 67.891 | −33.025 | 1.00 | 15.43 | A |
| ATOM | 5571 | CA | VAL | A | 708 | 25.093 | 67.896 | −31.719 | 1.00 | 14.68 | A |
| ATOM | 5572 | CB | VAL | A | 708 | 26.551 | 67.393 | −31.802 | 1.00 | 14.15 | A |
| ATOM | 5573 | CG1 | VAL | A | 708 | 27.195 | 67.426 | −30.417 | 1.00 | 13.61 | A |
| ATOM | 5574 | CG2 | VAL | A | 708 | 26.579 | 65.977 | −32.361 | 1.00 | 14.23 | A |
| ATOM | 5575 | C | VAL | A | 708 | 25.070 | 69.367 | −31.323 | 1.00 | 14.72 | A |
| ATOM | 5576 | O | VAL | A | 708 | 25.855 | 70.170 | −31.831 | 1.00 | 15.38 | A |
| ATOM | 5577 | N | HIS | A | 709 | 24.153 | 69.716 | −30.431 | 1.00 | 14.54 | A |
| ATOM | 5578 | CA | HIS | A | 709 | 23.990 | 71.101 | −30.011 | 1.00 | 15.48 | A |
| ATOM | 5579 | CB | HIS | A | 709 | 22.525 | 71.519 | −30.163 | 1.00 | 17.30 | A |
| ATOM | 5580 | CG | HIS | A | 709 | 22.015 | 71.462 | −31.568 | 1.00 | 20.53 | A |
| ATOM | 5581 | CD2 | HIS | A | 709 | 21.881 | 70.425 | −32.429 | 1.00 | 21.41 | A |
| ATOM | 5582 | ND1 | HIS | A | 709 | 21.546 | 72.574 | −32.233 | 1.00 | 22.57 | A |
| ATOM | 5583 | CE1 | HIS | A | 709 | 21.144 | 72.226 | −33.442 | 1.00 | 22.45 | A |
| ATOM | 5584 | NE2 | HIS | A | 709 | 21.337 | 70.927 | −33.587 | 1.00 | 21.98 | A |
| ATOM | 5585 | C | HIS | A | 709 | 24.421 | 71.403 | −28.586 | 1.00 | 14.93 | A |
| ATOM | 5586 | O | HIS | A | 709 | 24.001 | 70.731 | −27.647 | 1.00 | 14.53 | A |
| ATOM | 5587 | N | PHE | A | 710 | 25.258 | 72.424 | −28.435 | 1.00 | 13.24 | A |
| ATOM | 5588 | CA | PHE | A | 710 | 25.707 | 72.864 | −27.123 | 1.00 | 13.07 | A |
| ATOM | 5589 | CB | PHE | A | 710 | 27.122 | 73.446 | −27.192 | 1.00 | 13.58 | A |
| ATOM | 5590 | CG | PHE | A | 710 | 28.199 | 72.500 | −26.726 | 1.00 | 13.26 | A |
| ATOM | 5591 | CD1 | PHE | A | 710 | 29.349 | 72.310 | −27.484 | 1.00 | 14.61 | A |
| ATOM | 5592 | CD2 | PHE | A | 710 | 28.075 | 71.820 | −25.516 | 1.00 | 13.82 | A |
| ATOM | 5593 | CE1 | PHE | A | 710 | 30.364 | 71.457 | −27.043 | 1.00 | 14.19 | A |
| ATOM | 5594 | CE2 | PHE | A | 710 | 29.084 | 70.966 | −25.067 | 1.00 | 13.68 | A |
| ATOM | 5595 | CZ | PHE | A | 710 | 30.228 | 70.786 | −25.832 | 1.00 | 14.33 | A |
| ATOM | 5596 | C | PHE | A | 710 | 24.727 | 73.943 | −26.676 | 1.00 | 13.63 | A |
| ATOM | 5597 | O | PHE | A | 710 | 24.308 | 74.791 | −27.473 | 1.00 | 13.76 | A |
| ATOM | 5598 | N | LYS | A | 711 | 24.359 | 73.905 | −25.404 | 1.00 | 13.07 | A |
| ATOM | 5599 | CA | LYS | A | 711 | 23.427 | 74.871 | −24.847 | 1.00 | 14.24 | A |
| ATOM | 5600 | CB | LYS | A | 711 | 21.991 | 74.351 | −24.992 | 1.00 | 16.69 | A |
| ATOM | 5601 | CG | LYS | A | 711 | 20.926 | 75.268 | −24.410 | 1.00 | 18.89 | A |
| ATOM | 5602 | CD | LYS | A | 711 | 19.534 | 74.651 | −24.516 | 1.00 | 20.95 | A |
| ATOM | 5603 | CE | LYS | A | 711 | 19.101 | 74.480 | −25.964 | 1.00 | 21.44 | A |
| ATOM | 5604 | NZ | LYS | A | 711 | 17.747 | 73.861 | −26.067 | 1.00 | 22.65 | A |
| ATOM | 5605 | C | LYS | A | 711 | 23.751 | 75.089 | −23.378 | 1.00 | 14.02 | A |
| ATOM | 5606 | O | LYS | A | 711 | 24.144 | 74.155 | −22.678 | 1.00 | 14.38 | A |
| ATOM | 5607 | N | PHE | A | 712 | 23.609 | 76.325 | −22.917 | 1.00 | 12.94 | A |
| ATOM | 5608 | CA | PHE | A | 712 | 23.861 | 76.641 | −21.521 | 1.00 | 12.60 | A |
| ATOM | 5609 | CB | PHE | A | 712 | 24.859 | 77.799 | −21.384 | 1.00 | 13.19 | A |
| ATOM | 5610 | CG | PHE | A | 712 | 26.279 | 77.412 | −21.691 | 1.00 | 12.74 | A |
| ATOM | 5611 | CD1 | PHE | A | 712 | 26.761 | 77.449 | −22.996 | 1.00 | 12.90 | A |
| ATOM | 5612 | CD2 | PHE | A | 712 | 27.125 | 76.976 | −20.675 | 1.00 | 12.44 | A |
| ATOM | 5613 | CE1 | PHE | A | 712 | 28.066 | 77.057 | −23.288 | 1.00 | 13.16 | A |
| ATOM | 5614 | CE2 | PHE | A | 712 | 28.428 | 76.582 | −20.953 | 1.00 | 12.13 | A |
| ATOM | 5615 | CZ | PHE | A | 712 | 28.903 | 76.621 | −22.262 | 1.00 | 12.53 | A |
| ATOM | 5616 | C | PHE | A | 712 | 22.549 | 76.994 | −20.838 | 1.00 | 12.78 | A |
| ATOM | 5617 | O | PHE | A | 712 | 21.752 | 77.773 | −21.365 | 1.00 | 12.67 | A |
| ATOM | 5618 | N | LEU | A | 713 | 22.324 | 76.401 | −19.672 | 1.00 | 12.69 | A |
| ATOM | 5619 | CA | LEU | A | 713 | 21.105 | 76.643 | −18.916 | 1.00 | 12.88 | A |
| ATOM | 5620 | CB | LEU | A | 713 | 20.166 | 75.435 | −19.013 | 1.00 | 13.49 | A |
| ATOM | 5621 | CG | LEU | A | 713 | 19.790 | 74.962 | −20.421 | 1.00 | 13.72 | A |
| ATOM | 5622 | CD1 | LEU | A | 713 | 20.763 | 73.884 | −20.883 | 1.00 | 14.92 | A |
| ATOM | 5623 | CD2 | LEU | A | 713 | 18.365 | 74.407 | −20.414 | 1.00 | 14.75 | A |
| ATOM | 5624 | C | LEU | A | 713 | 21.447 | 76.915 | −17.461 | 1.00 | 13.49 | A |
| ATOM | 5625 | O | LEU | A | 713 | 22.605 | 76.806 | −17.054 | 1.00 | 13.14 | A |
| ATOM | 5626 | N | LYS | A | 714 | 20.441 | 77.274 | −16.674 | 1.00 | 13.55 | A |
| ATOM | 5627 | CA | LYS | A | 714 | 20.676 | 77.554 | −15.270 | 1.00 | 14.74 | A |
| ATOM | 5628 | CB | LYS | A | 714 | 20.715 | 79.066 | −15.034 | 1.00 | 17.89 | A |
| ATOM | 5629 | CG | LYS | A | 714 | 19.463 | 79.798 | −15.483 | 1.00 | 20.78 | A |
| ATOM | 5630 | CD | LYS | A | 714 | 19.519 | 81.279 | −15.113 | 1.00 | 23.52 | A |
| ATOM | 5631 | CE | LYS | A | 714 | 20.706 | 81.976 | −15.763 | 1.00 | 25.00 | A |
| ATOM | 5632 | NZ | LYS | A | 714 | 20.789 | 83.418 | −15.394 | 1.00 | 26.77 | A |
| ATOM | 5633 | C | LYS | A | 714 | 19.637 | 76.933 | −14.354 | 1.00 | 14.83 | A |
| ATOM | 5634 | O | LYS | A | 714 | 18.448 | 76.890 | −14.674 | 1.00 | 15.62 | A |
| ATOM | 5635 | N | TYR | A | 715 | 20.108 | 76.429 | −13.220 | 1.00 | 13.72 | A |
| ATOM | 5636 | CA | TYR | A | 715 | 19.234 | 75.853 | −12.212 | 1.00 | 12.72 | A |
| ATOM | 5637 | CB | TYR | A | 715 | 19.814 | 74.563 | −11.624 | 1.00 | 12.76 | A |
| ATOM | 5638 | CG | TYR | A | 715 | 19.709 | 73.345 | −12.507 | 1.00 | 11.86 | A |
| ATOM | 5639 | CD1 | TYR | A | 715 | 20.797 | 72.912 | −13.266 | 1.00 | 11.71 | A |
| ATOM | 5640 | CE1 | TYR | A | 715 | 20.717 | 71.766 | −14.053 | 1.00 | 10.85 | A |
| ATOM | 5641 | CD2 | TYR | A | 715 | 18.530 | 72.601 | −12.561 | 1.00 | 11.46 | A |
| ATOM | 5642 | CE2 | TYR | A | 715 | 18.438 | 71.453 | −13.345 | 1.00 | 12.03 | A |
| ATOM | 5643 | CZ | TYR | A | 715 | 19.535 | 71.039 | −14.088 | 1.00 | 11.99 | A |
| ATOM | 5644 | OH | TYR | A | 715 | 19.452 | 69.898 | −14.853 | 1.00 | 12.00 | A |
| ATOM | 5645 | C | TYR | A | 715 | 19.149 | 76.884 | −11.097 | 1.00 | 13.26 | A |
| ATOM | 5646 | O | TYR | A | 715 | 20.106 | 77.619 | −10.848 | 1.00 | 13.06 | A |
| ATOM | 5647 | N | GLY | A | 716 | 18.004 | 76.934 | −10.429 | 1.00 | 13.36 | A |
| ATOM | 5648 | CA | GLY | A | 716 | 17.832 | 77.871 | −9.338 | 1.00 | 13.61 | A |
| ATOM | 5649 | C | GLY | A | 716 | 17.746 | 77.146 | −8.010 | 1.00 | 15.27 | A |
| ATOM | 5650 | O | GLY | A | 716 | 18.096 | 75.965 | −7.906 | 1.00 | 15.61 | A |
| ATOM | 5651 | N | VAL | A | 717 | 17.270 | 77.858 | −6.997 | 1.00 | 15.67 | A |
| ATOM | 5652 | CA | VAL | A | 717 | 17.123 | 77.316 | −5.655 | 1.00 | 16.91 | A |
| ATOM | 5653 | CB | VAL | A | 717 | 18.060 | 78.055 | −4.672 | 1.00 | 16.38 | A |
| ATOM | 5654 | CG1 | VAL | A | 717 | 17.825 | 77.577 | −3.252 | 1.00 | 17.56 | A |
| ATOM | 5655 | CG2 | VAL | A | 717 | 19.512 | 77.823 | −5.076 | 1.00 | 16.85 | A |
| ATOM | 5656 | C | VAL | A | 717 | 15.670 | 77.474 | −5.217 | 1.00 | 17.83 | A |
| ATOM | 5657 | O | VAL | A | 717 | 14.981 | 78.398 | −5.650 | 1.00 | 18.06 | A |
| ATOM | 5658 | N | ARG | A | 718 | 15.204 | 76.569 | −4.364 | 1.00 | 18.88 | A |
| ATOM | 5659 | CA | ARG | A | 718 | 13.826 | 76.607 | −3.889 | 1.00 | 20.57 | A |
| ATOM | 5660 | CB | ARG | A | 718 | 13.470 | 75.274 | −3.232 | 1.00 | 19.83 | A |
| ATOM | 5661 | CG | ARG | A | 718 | 13.580 | 74.112 | −4.198 | 1.00 | 19.14 | A |
| ATOM | 5662 | CD | ARG | A | 718 | 13.483 | 72.765 | −3.511 | 1.00 | 18.32 | A |
| ATOM | 5663 | NE | ARG | A | 718 | 13.768 | 71.695 | −4.460 | 1.00 | 17.75 | A |
| ATOM | 5664 | CZ | ARG | A | 718 | 13.765 | 70.401 | −4.161 | 1.00 | 17.53 | A |
| ATOM | 5665 | NH1 | ARG | A | 718 | 13.485 | 69.999 | −2.928 | 1.00 | 17.28 | A |
| ATOM | 5666 | NH2 | ARG | A | 718 | 14.048 | 69.508 | −5.101 | 1.00 | 17.74 | A |
| ATOM | 5667 | C | ARG | A | 718 | 13.562 | 77.753 | −2.924 | 1.00 | 22.46 | A |
| ATOM | 5668 | O | ARG | A | 718 | 14.398 | 78.078 | −2.085 | 1.00 | 22.85 | A |
| ATOM | 5669 | N | SER | A | 719 | 12.391 | 78.365 | −3.059 | 1.00 | 24.66 | A |
| ATOM | 5670 | CA | SER | A | 719 | 12.000 | 79.475 | −2.201 | 1.00 | 27.21 | A |
| ATOM | 5671 | CB | SER | A | 719 | 11.082 | 80.431 | −2.966 | 1.00 | 27.33 | A |
| ATOM | 5672 | OG | SER | A | 719 | 9.936 | 79.751 | −3.447 | 1.00 | 28.77 | A |
| ATOM | 5673 | C | SER | A | 719 | 11.280 | 78.942 | −0.969 | 1.00 | 28.70 | A |
| ATOM | 5674 | O | SER | A | 719 | 10.976 | 79.690 | −0.039 | 1.00 | 29.02 | A |
| ATOM | 5675 | N | HIS | A | 720 | 11.009 | 77.641 | −0.977 | 1.00 | 30.58 | A |
| ATOM | 5676 | CA | HIS | A | 720 | 10.328 | 76.981 | 0.128 | 1.00 | 32.12 | A |
| ATOM | 5677 | CB | HIS | A | 720 | 8.873 | 76.683 | −0.246 | 1.00 | 34.89 | A |
| ATOM | 5678 | CG | HIS | A | 720 | 8.133 | 77.865 | −0.791 | 1.00 | 37.77 | A |
| ATOM | 5679 | CD2 | HIS | A | 720 | 7.534 | 78.066 | −1.989 | 1.00 | 38.94 | A |
| ATOM | 5680 | ND1 | HIS | A | 720 | 7.945 | 79.024 | −0.069 | 1.00 | 39.13 | A |
| ATOM | 5681 | CE1 | HIS | A | 720 | 7.262 | 79.888 | −0.799 | 1.00 | 39.81 | A |
| ATOM | 5682 | NE2 | HIS | A | 720 | 7.001 | 79.331 | −1.968 | 1.00 | 39.76 | A |
| ATOM | 5683 | C | HIS | A | 720 | 11.042 | 75.670 | 0.443 | 1.00 | 31.40 | A |
| ATOM | 5684 | O | HIS | A | 720 | 11.506 | 74.977 | −0.462 | 1.00 | 31.75 | A |
| ATOM | 5685 | N | GLY | A | 721 | 11.133 | 75.337 | 1.726 | 1.00 | 30.13 | A |
| ATOM | 5686 | CA | GLY | A | 721 | 11.782 | 74.100 | 2.119 | 1.00 | 28.34 | A |
| ATOM | 5687 | C | GLY | A | 721 | 13.299 | 74.148 | 2.131 | 1.00 | 26.81 | A |
| ATOM | 5688 | O | GLY | A | 721 | 13.900 | 75.210 | 2.278 | 1.00 | 26.70 | A |
| ATOM | 5689 | N | ASP | A | 722 | 13.912 | 72.980 | 1.964 | 1.00 | 24.89 | A |
| ATOM | 5690 | CA | ASP | A | 722 | 15.365 | 72.837 | 1.970 | 1.00 | 22.79 | A |
| ATOM | 5691 | CB | ASP | A | 722 | 15.726 | 71.351 | 1.955 | 1.00 | 21.90 | A |
| ATOM | 5692 | CG | ASP | A | 722 | 15.174 | 70.607 | 3.158 | 1.00 | 22.17 | A |
| ATOM | 5693 | OD1 | ASP | A | 722 | 15.057 | 69.367 | 3.086 | 1.00 | 21.82 | A |
| ATOM | 5694 | OD2 | ASP | A | 722 | 14.865 | 71.261 | 4.179 | 1.00 | 21.77 | A |
| ATOM | 5695 | C | ASP | A | 722 | 16.045 | 73.544 | 0.801 | 1.00 | 21.36 | A |
| ATOM | 5696 | O | ASP | A | 722 | 15.605 | 73.440 | −0.343 | 1.00 | 20.27 | A |
| ATOM | 5697 | N | ARG | A | 723 | 17.127 | 74.257 | 1.102 | 1.00 | 20.52 | A |
| ATOM | 5698 | CA | ARG | A | 723 | 17.880 | 74.988 | 0.089 | 1.00 | 20.03 | A |
| ATOM | 5699 | CB | ARG | A | 723 | 18.196 | 76.408 | 0.572 | 1.00 | 22.97 | A |
| ATOM | 5700 | CG | ARG | A | 723 | 17.039 | 77.396 | 0.483 | 1.00 | 27.93 | A |
| ATOM | 5701 | CD | ARG | A | 723 | 15.997 | 77.176 | 1.566 | 1.00 | 31.86 | A |
| ATOM | 5702 | NE | ARG | A | 723 | 14.917 | 78.157 | 1.468 | 1.00 | 35.16 | A |
| ATOM | 5703 | CZ | ARG | A | 723 | 13.956 | 78.314 | 2.374 | 1.00 | 36.54 | A |
| ATOM | 5704 | NH1 | ARG | A | 723 | 13.931 | 77.555 | 3.462 | 1.00 | 37.74 | A |
| ATOM | 5705 | NH2 | ARG | A | 723 | 13.019 | 79.236 | 2.194 | 1.00 | 37.45 | A |
| ATOM | 5706 | C | ARG | A | 723 | 19.188 | 74.300 | −0.286 | 1.00 | 18.02 | A |
| ATOM | 5707 | O | ARG | A | 723 | 19.798 | 73.602 | 0.528 | 1.00 | 16.96 | A |
| ATOM | 5708 | N | SER | A | 724 | 19.612 | 74.508 | −1.528 | 1.00 | 16.11 | A |
| ATOM | 5709 | CA | SER | A | 724 | 20.857 | 73.940 | −2.017 | 1.00 | 14.57 | A |
| ATOM | 5710 | CB | SER | A | 724 | 20.993 | 74.174 | −3.521 | 1.00 | 14.09 | A |
| ATOM | 5711 | OG | SER | A | 724 | 19.919 | 73.589 | −4.227 | 1.00 | 13.67 | A |
| ATOM | 5712 | C | SER | A | 724 | 22.023 | 74.610 | −1.302 | 1.00 | 13.51 | A |
| ATOM | 5713 | O | SER | A | 724 | 21.949 | 75.786 | −0.932 | 1.00 | 14.47 | A |
| ATOM | 5714 | N | GLY | A | 725 | 23.101 | 73.855 | −1.120 | 1.00 | 12.12 | A |
| ATOM | 5715 | CA | GLY | A | 725 | 24.286 | 74.378 | −0.466 | 1.00 | 10.74 | A |
| ATOM | 5716 | C | GLY | A | 725 | 25.497 | 73.661 | −1.030 | 1.00 | 9.87 | A |
| ATOM | 5717 | O | GLY | A | 725 | 25.408 | 73.054 | −2.093 | 1.00 | 10.42 | A |
| ATOM | 5718 | N | ALA | A | 726 | 26.623 | 73.718 | −0.325 | 1.00 | 10.23 | A |
| ATOM | 5719 | CA | ALA | A | 726 | 27.844 | 73.068 | −0.785 | 1.00 | 9.51 | A |
| ATOM | 5720 | CB | ALA | A | 726 | 28.981 | 73.331 | 0.200 | 1.00 | 10.07 | A |
| ATOM | 5721 | C | ALA | A | 726 | 27.696 | 71.564 | −1.004 | 1.00 | 10.03 | A |
| ATOM | 5722 | O | ALA | A | 726 | 28.359 | 70.995 | −1.876 | 1.00 | 9.75 | A |
| ATOM | 5723 | N | TYR | A | 727 | 26.839 | 70.918 | −0.216 | 1.00 | 9.25 | A |
| ATOM | 5724 | CA | TYR | A | 727 | 26.648 | 69.475 | −0.343 | 1.00 | 9.04 | A |
| ATOM | 5725 | CB | TYR | A | 727 | 26.434 | 68.811 | 1.025 | 1.00 | 9.17 | A |
| ATOM | 5726 | CG | TYR | A | 727 | 27.431 | 69.165 | 2.099 | 1.00 | 9.57 | A |
| ATOM | 5727 | CD1 | TYR | A | 727 | 27.306 | 70.345 | 2.833 | 1.00 | 10.21 | A |
| ATOM | 5728 | CE1 | TYR | A | 727 | 28.201 | 70.656 | 3.851 | 1.00 | 10.74 | A |
| ATOM | 5729 | CD2 | TYR | A | 727 | 28.486 | 68.305 | 2.407 | 1.00 | 10.18 | A |
| ATOM | 5730 | CE2 | TYR | A | 727 | 29.386 | 68.609 | 3.422 | 1.00 | 10.51 | A |
| ATOM | 5731 | CZ | TYR | A | 727 | 29.237 | 69.783 | 4.142 | 1.00 | 10.60 | A |
| ATOM | 5732 | OH | TYR | A | 727 | 30.101 | 70.075 | 5.170 | 1.00 | 11.18 | A |
| ATOM | 5733 | C | TYR | A | 727 | 25.456 | 69.034 | −1.200 | 1.00 | 9.43 | A |
| ATOM | 5734 | O | TYR | A | 727 | 25.570 | 68.247 | −2.096 | 1.00 | 9.80 | A |
| ATOM | 5735 | N | LEU | A | 728 | 24.314 | 69.695 | −0.907 | 1.00 | 9.86 | A |
| ATOM | 5736 | CA | LEU | A | 728 | 23.060 | 69.371 | −1.576 | 1.00 | 10.29 | A |
| ATOM | 5737 | CB | LEU | A | 728 | 21.906 | 69.519 | −0.581 | 1.00 | 10.54 | A |
| ATOM | 5738 | CG | LEU | A | 728 | 22.078 | 68.918 | 0.817 | 1.00 | 9.48 | A |
| ATOM | 5739 | CD1 | LEU | A | 728 | 20.799 | 69.151 | 1.618 | 1.00 | 11.57 | A |
| ATOM | 5740 | CD2 | LEU | A | 728 | 22.398 | 67.434 | 0.725 | 1.00 | 10.76 | A |
| ATOM | 5741 | C | LEU | A | 728 | 22.687 | 70.135 | −2.836 | 1.00 | 10.59 | A |
| ATOM | 5742 | O | LEU | A | 728 | 22.895 | 71.343 | −2.938 | 1.00 | 11.53 | A |
| ATOM | 5743 | N | PHE | A | 729 | 22.115 | 69.399 | −3.786 | 1.00 | 11.08 | A |
| ATOM | 5744 | CA | PHE | A | 729 | 21.625 | 69.956 | −5.040 | 1.00 | 11.31 | A |
| ATOM | 5745 | CB | PHE | A | 729 | 22.157 | 69.154 | −6.233 | 1.00 | 10.97 | A |
| ATOM | 5746 | CG | PHE | A | 729 | 21.677 | 69.653 | −7.576 | 1.00 | 10.34 | A |
| ATOM | 5747 | CD1 | PHE | A | 729 | 21.609 | 68.783 | −8.659 | 1.00 | 10.55 | A |
| ATOM | 5748 | CD2 | PHE | A | 729 | 21.322 | 70.990 | −7.766 | 1.00 | 11.51 | A |
| ATOM | 5749 | CE1 | PHE | A | 729 | 21.194 | 69.228 | −9.910 | 1.00 | 10.69 | A |
| ATOM | 5750 | CE2 | PHE | A | 729 | 20.906 | 71.447 | −9.020 | 1.00 | 10.06 | A |
| ATOM | 5751 | CZ | PHE | A | 729 | 20.843 | 70.563 | −10.092 | 1.00 | 11.02 | A |
| ATOM | 5752 | C | PHE | A | 729 | 20.109 | 69.792 | −4.940 | 1.00 | 11.73 | A |
| ATOM | 5753 | O | PHE | A | 729 | 19.589 | 68.682 | −5.040 | 1.00 | 11.22 | A |
| ATOM | 5754 | N | LEU | A | 730 | 19.408 | 70.900 | −4.725 | 1.00 | 12.20 | A |
| ATOM | 5755 | CA | LEU | A | 730 | 17.955 | 70.886 | −4.592 | 1.00 | 13.36 | A |
| ATOM | 5756 | CB | LEU | A | 730 | 17.564 | 71.222 | −3.151 | 1.00 | 13.41 | A |
| ATOM | 5757 | CG | LEU | A | 730 | 17.990 | 70.185 | −2.104 | 1.00 | 13.71 | A |
| ATOM | 5758 | CD1 | LEU | A | 730 | 17.919 | 70.781 | −0.712 | 1.00 | 13.88 | A |
| ATOM | 5759 | CD2 | LEU | A | 730 | 17.095 | 68.961 | −2.208 | 1.00 | 14.18 | A |
| ATOM | 5760 | C | LEU | A | 730 | 17.371 | 71.910 | −5.552 | 1.00 | 13.24 | A |
| ATOM | 5761 | O | LEU | A | 730 | 16.881 | 72.961 | −5.139 | 1.00 | 13.97 | A |
| ATOM | 5762 | N | PRO | A | 731 | 17.414 | 71.607 | −6.857 | 1.00 | 13.87 | A |
| ATOM | 5763 | CD | PRO | A | 731 | 17.825 | 70.324 | −7.455 | 1.00 | 13.91 | A |
| ATOM | 5764 | CA | PRO | A | 731 | 16.893 | 72.513 | −7.882 | 1.00 | 14.19 | A |
| ATOM | 5765 | CB | PRO | A | 731 | 17.232 | 71.788 | −9.177 | 1.00 | 13.69 | A |
| ATOM | 5766 | CG | PRO | A | 731 | 17.111 | 70.346 | −8.782 | 1.00 | 13.97 | A |
| ATOM | 5767 | C | PRO | A | 731 | 15.409 | 72.819 | −7.769 | 1.00 | 15.00 | A |
| ATOM | 5768 | O | PRO | A | 731 | 14.627 | 72.015 | −7.257 | 1.00 | 14.35 | A |
| ATOM | 5769 | N | ASN | A | 732 | 15.033 | 73.998 | −8.249 | 1.00 | 16.03 | A |
| ATOM | 5770 | CA | ASN | A | 732 | 13.640 | 74.412 | −8.242 | 1.00 | 17.20 | A |
| ATOM | 5771 | CB | ASN | A | 732 | 13.531 | 75.915 | −7.954 | 1.00 | 18.72 | A |
| ATOM | 5772 | CG | ASN | A | 732 | 14.202 | 76.766 | −9.013 | 1.00 | 19.71 | A |
| ATOM | 5773 | OD1 | ASN | A | 732 | 15.289 | 76.446 | −9.485 | 1.00 | 21.05 | A |
| ATOM | 5774 | ND2 | ASN | A | 732 | 13.559 | 77.871 | −9.380 | 1.00 | 21.04 | A |
| ATOM | 5775 | C | ASN | A | 732 | 13.084 | 74.075 | −9.620 | 1.00 | 16.86 | A |
| ATOM | 5776 | O | ASN | A | 732 | 12.561 | 74.936 | −10.330 | 1.00 | 17.62 | A |
| ATOM | 5777 | N | GLY | A | 733 | 13.222 | 72.806 | −9.993 | 1.00 | 15.74 | A |
| ATOM | 5778 | CA | GLY | A | 733 | 12.737 | 72.343 | −11.281 | 1.00 | 16.20 | A |
| ATOM | 5779 | C | GLY | A | 733 | 13.820 | 72.251 | −12.340 | 1.00 | 15.85 | A |
| ATOM | 5780 | O | GLY | A | 733 | 14.979 | 72.600 | −12.084 | 1.00 | 15.63 | A |
| ATOM | 5781 | N | PRO | A | 734 | 13.475 | 71.774 | −13.546 | 1.00 | 15.55 | A |
| ATOM | 5782 | CD | PRO | A | 734 | 12.159 | 71.236 | −13.935 | 1.00 | 16.13 | A |
| ATOM | 5783 | CA | PRO | A | 734 | 14.433 | 71.643 | −14.646 | 1.00 | 15.56 | A |
| ATOM | 5784 | CB | PRO | A | 734 | 13.565 | 71.152 | −15.801 | 1.00 | 16.06 | A |
| ATOM | 5785 | CG | PRO | A | 734 | 12.510 | 70.353 | −15.106 | 1.00 | 17.37 | A |
| ATOM | 5786 | C | PRO | A | 734 | 15.091 | 72.983 | −14.953 | 1.00 | 15.30 | A |
| ATOM | 5787 | O | PRO | A | 734 | 14.519 | 74.042 | −14.691 | 1.00 | 14.97 | A |
| ATOM | 5788 | N | ALA | A | 735 | 16.288 | 72.926 | −15.525 | 1.00 | 15.31 | A |
| ATOM | 5789 | CA | ALA | A | 735 | 17.041 | 74.128 | −15.856 | 1.00 | 15.45 | A |
| ATOM | 5790 | CB | ALA | A | 735 | 18.440 | 73.745 | −16.324 | 1.00 | 15.38 | A |
| ATOM | 5791 | C | ALA | A | 735 | 16.355 | 74.991 | −16.910 | 1.00 | 16.40 | A |
| ATOM | 5792 | O | ALA | A | 735 | 15.641 | 74.488 | −17.777 | 1.00 | 16.52 | A |
| ATOM | 5793 | N | SER | A | 736 | 16.584 | 76.298 | −16.816 | 1.00 | 17.32 | A |
| ATOM | 5794 | CA | SER | A | 736 | 16.019 | 77.265 | −17.747 | 1.00 | 18.50 | A |
| ATOM | 5795 | CB | SER | A | 736 | 15.387 | 78.427 | −16.978 | 1.00 | 19.12 | A |
| ATOM | 5796 | OG | SER | A | 736 | 14.401 | 77.959 | −16.074 | 1.00 | 22.89 | A |
| ATOM | 5797 | C | SER | A | 736 | 17.158 | 77.779 | −18.622 | 1.00 | 19.11 | A |
| ATOM | 5798 | O | SER | A | 736 | 18.259 | 78.019 | −18.134 | 1.00 | 18.19 | A |
| ATOM | 5799 | N | PRO | A | 737 | 16.907 | 77.955 | −19.928 | 1.00 | 19.88 | A |
| ATOM | 5800 | CD | PRO | A | 737 | 15.659 | 77.671 | −20.656 | 1.00 | 20.64 | A |
| ATOM | 5801 | CA | PRO | A | 737 | 17.943 | 78.443 | −20.845 | 1.00 | 20.87 | A |
| ATOM | 5802 | CB | PRO | A | 737 | 17.215 | 78.502 | −22.188 | 1.00 | 21.48 | A |
| ATOM | 5803 | CG | PRO | A | 737 | 16.162 | 77.441 | −22.058 | 1.00 | 21.45 | A |
| ATOM | 5804 | C | PRO | A | 737 | 18.504 | 79.803 | −20.441 | 1.00 | 21.50 | A |
| ATOM | 5805 | O | PRO | A | 737 | 17.764 | 80.685 | −20.004 | 1.00 | 21.33 | A |
| ATOM | 5806 | N | VAL | A | 738 | 19.816 | 79.966 | −20.577 | 1.00 | 22.39 | A |
| ATOM | 5807 | CA | VAL | A | 738 | 20.456 | 81.234 | −20.254 | 1.00 | 23.03 | A |
| ATOM | 5808 | CB | VAL | A | 738 | 21.993 | 81.082 | −20.119 | 1.00 | 22.73 | A |
| ATOM | 5809 | CG1 | VAL | A | 738 | 22.645 | 82.456 | −19.993 | 1.00 | 22.43 | A |
| ATOM | 5810 | CG2 | VAL | A | 738 | 22.332 | 80.229 | −18.903 | 1.00 | 21.84 | A |
| ATOM | 5811 | C | VAL | A | 738 | 20.162 | 82.191 | −21.407 | 1.00 | 24.47 | A |
| ATOM | 5812 | O | VAL | A | 738 | 20.342 | 81.834 | −22.570 | 1.00 | 24.12 | A |
| ATOM | 5813 | N | GLU | A | 739 | 19.692 | 83.392 | −21.088 | 1.00 | 25.62 | A |
| ATOM | 5814 | CA | GLU | A | 739 | 19.398 | 84.380 | −22.121 | 1.00 | 26.93 | A |
| ATOM | 5815 | CB | GLU | A | 739 | 18.604 | 85.546 | −21.531 | 1.00 | 28.81 | A |
| ATOM | 5816 | CG | GLU | A | 739 | 17.210 | 85.155 | −21.066 | 1.00 | 32.07 | A |
| ATOM | 5817 | CD | GLU | A | 739 | 16.327 | 84.675 | −22.205 | 1.00 | 33.84 | A |
| ATOM | 5818 | OE1 | GLU | A | 739 | 15.194 | 84.226 | −21.932 | 1.00 | 35.19 | A |
| ATOM | 5819 | OE2 | GLU | A | 739 | 16.763 | 84.749 | −23.374 | 1.00 | 34.81 | A |
| ATOM | 5820 | C | GLU | A | 739 | 20.731 | 84.866 | −22.671 | 1.00 | 26.25 | A |
| ATOM | 5821 | O | GLU | A | 739 | 21.498 | 85.525 | −21.971 | 1.00 | 25.93 | A |
| ATOM | 5822 | N | LEU | A | 740 | 20.996 | 84.537 | −23.931 | 1.00 | 26.09 | A |
| ATOM | 5823 | CA | LEU | A | 740 | 22.257 | 84.889 | −24.574 | 1.00 | 25.99 | A |
| ATOM | 5824 | CB | LEU | A | 740 | 22.631 | 83.803 | −25.583 | 1.00 | 26.19 | A |
| ATOM | 5825 | CG | LEU | A | 740 | 22.563 | 82.362 | −25.073 | 1.00 | 25.97 | A |
| ATOM | 5826 | CD1 | LEU | A | 740 | 22.900 | 81.411 | −26.208 | 1.00 | 25.89 | A |
| ATOM | 5827 | CD2 | LEU | A | 740 | 23.525 | 82.179 | −23.905 | 1.00 | 26.08 | A |
| ATOM | 5828 | C | LEU | A | 740 | 22.304 | 86.240 | −25.273 | 1.00 | 26.32 | A |
| ATOM | 5829 | O | LEU | A | 740 | 23.384 | 86.795 | −25.463 | 1.00 | 25.85 | A |
| ATOM | 5830 | N | GLY | A | 741 | 21.146 | 86.767 | −25.656 | 1.00 | 26.45 | A |
| ATOM | 5831 | CA | GLY | A | 741 | 21.127 | 88.040 | −26.353 | 1.00 | 26.72 | A |
| ATOM | 5832 | C | GLY | A | 741 | 21.822 | 87.872 | −27.692 | 1.00 | 26.42 | A |
| ATOM | 5833 | O | GLY | A | 741 | 21.597 | 86.883 | −28.387 | 1.00 | 27.17 | A |
| ATOM | 5834 | N | GLN | A | 742 | 22.666 | 88.831 | −28.060 | 1.00 | 26.36 | A |
| ATOM | 5835 | CA | GLN | A | 742 | 23.407 | 88.766 | −29.318 | 1.00 | 25.57 | A |
| ATOM | 5836 | CB | GLN | A | 742 | 23.036 | 89.953 | −30.214 | 1.00 | 29.15 | A |
| ATOM | 5837 | CG | GLN | A | 742 | 21.557 | 89.999 | −30.588 | 1.00 | 33.16 | A |
| ATOM | 5838 | CD | GLN | A | 742 | 21.200 | 91.197 | −31.449 | 1.00 | 35.56 | A |
| ATOM | 5839 | OE1 | GLN | A | 742 | 21.723 | 91.366 | −32.551 | 1.00 | 37.11 | A |
| ATOM | 5840 | NE2 | GLN | A | 742 | 20.299 | 92.036 | −30.948 | 1.00 | 36.79 | A |
| ATOM | 5841 | C | GLN | A | 742 | 24.895 | 88.800 | −28.977 | 1.00 | 23.07 | A |
| ATOM | 5842 | O | GLN | A | 742 | 25.555 | 89.829 | −29.113 | 1.00 | 22.79 | A |
| ATOM | 5843 | N | PRO | A | 743 | 25.444 | 87.660 | −28.530 | 1.00 | 20.48 | A |
| ATOM | 5844 | CD | PRO | A | 743 | 24.783 | 86.343 | −28.449 | 1.00 | 19.61 | A |
| ATOM | 5845 | CA | PRO | A | 743 | 26.857 | 87.558 | −28.157 | 1.00 | 18.31 | A |
| ATOM | 5846 | CB | PRO | A | 743 | 26.946 | 86.164 | −27.544 | 1.00 | 18.28 | A |
| ATOM | 5847 | CG | PRO | A | 743 | 25.961 | 85.390 | −28.358 | 1.00 | 18.15 | A |
| ATOM | 5848 | C | PRO | A | 743 | 27.871 | 87.760 | −29.277 | 1.00 | 16.88 | A |
| ATOM | 5849 | O | PRO | A | 743 | 27.589 | 87.516 | −30.449 | 1.00 | 16.70 | A |
| ATOM | 5850 | N | VAL | A | 744 | 29.058 | 88.218 | −28.893 | 1.00 | 14.74 | A |
| ATOM | 5851 | CA | VAL | A | 744 | 30.139 | 88.437 | −29.840 | 1.00 | 13.49 | A |
| ATOM | 5852 | CB | VAL | A | 744 | 31.149 | 89.464 | −29.296 | 1.00 | 12.90 | A |
| ATOM | 5853 | CG1 | VAL | A | 744 | 32.284 | 89.655 | −30.287 | 1.00 | 12.99 | A |
| ATOM | 5854 | CG2 | VAL | A | 744 | 30.442 | 90.789 | −29.030 | 1.00 | 13.89 | A |
| ATOM | 5855 | C | VAL | A | 744 | 30.842 | 87.105 | −30.070 | 1.00 | 12.96 | A |
| ATOM | 5856 | O | VAL | A | 744 | 31.183 | 86.401 | −29.117 | 1.00 | 12.48 | A |
| ATOM | 5857 | N | VAL | A | 745 | 31.041 | 86.763 | −31.338 | 1.00 | 11.61 | A |
| ATOM | 5858 | CA | VAL | A | 745 | 31.687 | 85.514 | −31.717 | 1.00 | 11.18 | A |
| ATOM | 5859 | CB | VAL | A | 745 | 30.755 | 84.672 | −32.615 | 1.00 | 10.82 | A |
| ATOM | 5860 | CG1 | VAL | A | 745 | 31.418 | 83.359 | −32.981 | 1.00 | 11.01 | A |
| ATOM | 5861 | CG2 | VAL | A | 745 | 29.436 | 84.430 | −31.904 | 1.00 | 10.80 | A |
| ATOM | 5862 | C | VAL | A | 745 | 32.992 | 85.765 | −32.463 | 1.00 | 11.19 | A |
| ATOM | 5863 | O | VAL | A | 745 | 33.041 | 86.564 | −33.403 | 1.00 | 11.51 | A |
| ATOM | 5864 | N | LEU | A | 746 | 34.047 | 85.077 | −32.041 | 1.00 | 10.48 | A |
| ATOM | 5865 | CA | LEU | A | 746 | 35.359 | 85.208 | −32.662 | 1.00 | 10.17 | A |
| ATOM | 5866 | CB | LEU | A | 746 | 36.409 | 85.575 | −31.611 | 1.00 | 11.05 | A |
| ATOM | 5867 | CG | LEU | A | 746 | 37.865 | 85.599 | −32.087 | 1.00 | 11.54 | A |
| ATOM | 5868 | CD1 | LEU | A | 746 | 38.050 | 86.671 | −33.153 | 1.00 | 13.05 | A |
| ATOM | 5869 | CD2 | LEU | A | 746 | 38.779 | 85.863 | −30.900 | 1.00 | 13.86 | A |
| ATOM | 5870 | C | LEU | A | 746 | 35.761 | 83.908 | −33.347 | 1.00 | 10.30 | A |
| ATOM | 5871 | O | LEU | A | 746 | 35.820 | 82.847 | −32.718 | 1.00 | 10.42 | A |
| ATOM | 5872 | N | VAL | A | 747 | 36.044 | 83.992 | −34.639 | 1.00 | 10.07 | A |
| ATOM | 5873 | CA | VAL | A | 747 | 36.435 | 82.824 | −35.412 | 1.00 | 10.40 | A |
| ATOM | 5874 | CB | VAL | A | 747 | 35.535 | 82.644 | −36.658 | 1.00 | 10.85 | A |
| ATOM | 5875 | CG1 | VAL | A | 747 | 35.903 | 81.361 | −37.385 | 1.00 | 10.29 | A |
| ATOM | 5876 | CG2 | VAL | A | 747 | 34.079 | 82.631 | −36.248 | 1.00 | 10.45 | A |
| ATOM | 5877 | C | VAL | A | 747 | 37.872 | 82.964 | −35.878 | 1.00 | 11.25 | A |
| ATOM | 5878 | O | VAL | A | 747 | 38.214 | 83.908 | −36.592 | 1.00 | 11.81 | A |
| ATOM | 5879 | N | THR | A | 748 | 38.718 | 82.033 | −35.458 | 1.00 | 10.82 | A |
| ATOM | 5880 | CA | THR | A | 748 | 40.111 | 82.046 | −35.866 | 1.00 | 12.00 | A |
| ATOM | 5881 | CB | THR | A | 748 | 41.047 | 82.030 | −34.646 | 1.00 | 12.71 | A |
| ATOM | 5882 | OG1 | THR | A | 748 | 40.806 | 83.201 | −33.853 | 1.00 | 12.84 | A |
| ATOM | 5883 | CG2 | THR | A | 748 | 42.502 | 82.010 | −35.089 | 1.00 | 13.11 | A |
| ATOM | 5884 | C | THR | A | 748 | 40.320 | 80.804 | −36.715 | 1.00 | 12.53 | A |
| ATOM | 5885 | O | THR | A | 748 | 40.056 | 79.689 | −36.273 | 1.00 | 12.33 | A |
| ATOM | 5886 | N | LYS | A | 749 | 40.768 | 81.004 | −37.949 | 1.00 | 11.99 | A |
| ATOM | 5887 | CA | LYS | A | 749 | 40.989 | 79.898 | −38.868 | 1.00 | 12.97 | A |
| ATOM | 5888 | CB | LYS | A | 749 | 40.162 | 80.099 | −40.140 | 1.00 | 14.41 | A |
| ATOM | 5889 | CG | LYS | A | 749 | 40.361 | 79.009 | −41.176 | 1.00 | 17.12 | A |
| ATOM | 5890 | CD | LYS | A | 749 | 39.465 | 79.222 | −42.384 | 1.00 | 19.62 | A |
| ATOM | 5891 | CE | LYS | A | 749 | 39.708 | 78.154 | −43.438 | 1.00 | 21.33 | A |
| ATOM | 5892 | NZ | LYS | A | 749 | 38.867 | 78.377 | −44.648 | 1.00 | 24.55 | A |
| ATOM | 5893 | C | LYS | A | 749 | 42.457 | 79.758 | −39.230 | 1.00 | 12.94 | A |
| ATOM | 5894 | O | LYS | A | 749 | 43.062 | 80.671 | −39.797 | 1.00 | 12.59 | A |
| ATOM | 5895 | N | GLY | A | 750 | 43.023 | 78.603 | −38.903 | 1.00 | 12.38 | A |
| ATOM | 5896 | CA | GLY | A | 750 | 44.420 | 78.351 | −39.196 | 1.00 | 12.98 | A |
| ATOM | 5897 | C | GLY | A | 750 | 44.621 | 76.977 | −39.796 | 1.00 | 13.35 | A |
| ATOM | 5898 | O | GLY | A | 750 | 43.726 | 76.130 | −39.752 | 1.00 | 14.37 | A |
| ATOM | 5899 | N | LYS | A | 751 | 45.802 | 76.755 | −40.362 | 1.00 | 13.85 | A |
| ATOM | 5900 | CA | LYS | A | 751 | 46.122 | 75.474 | −40.976 | 1.00 | 14.48 | A |
| ATOM | 5901 | CB | LYS | A | 751 | 47.423 | 75.588 | −41.774 | 1.00 | 17.11 | A |
| ATOM | 5902 | CG | LYS | A | 751 | 47.355 | 76.562 | −42.939 | 1.00 | 21.85 | A |
| ATOM | 5903 | CD | LYS | A | 751 | 46.358 | 76.094 | −43.990 | 1.00 | 25.31 | A |
| ATOM | 5904 | CE | LYS | A | 751 | 46.339 | 77.030 | −45.190 | 1.00 | 26.61 | A |
| ATOM | 5905 | NZ | LYS | A | 751 | 45.405 | 76.557 | −46.249 | 1.00 | 28.25 | A |
| ATOM | 5906 | C | LYS | A | 751 | 46.264 | 74.370 | −39.933 | 1.00 | 13.47 | A |
| ATOM | 5907 | O | LYS | A | 751 | 45.878 | 73.228 | −40.174 | 1.00 | 13.67 | A |
| ATOM | 5908 | N | LEU | A | 752 | 46.812 | 74.718 | −38.774 | 1.00 | 12.85 | A |
| ATOM | 5909 | CA | LEU | A | 752 | 47.018 | 73.741 | −37.711 | 1.00 | 12.63 | A |
| ATOM | 5910 | CB | LEU | A | 752 | 48.386 | 73.957 | −37.059 | 1.00 | 12.95 | A |
| ATOM | 5911 | CG | LEU | A | 752 | 49.612 | 73.940 | −37.976 | 1.00 | 13.92 | A |
| ATOM | 5912 | CD1 | LEU | A | 752 | 50.867 | 74.114 | −37.137 | 1.00 | 14.66 | A |
| ATOM | 5913 | CD2 | LEU | A | 752 | 49.663 | 72.635 | −38.752 | 1.00 | 15.60 | A |
| ATOM | 5914 | C | LEU | A | 752 | 45.947 | 73.781 | −36.627 | 1.00 | 12.32 | A |
| ATOM | 5915 | O | LEU | A | 752 | 45.616 | 72.756 | −36.033 | 1.00 | 11.49 | A |
| ATOM | 5916 | N | GLU | A | 753 | 45.403 | 74.964 | −36.372 | 1.00 | 11.65 | A |
| ATOM | 5917 | CA | GLU | A | 753 | 44.400 | 75.109 | −35.332 | 1.00 | 11.90 | A |
| ATOM | 5918 | CB | GLU | A | 753 | 45.087 | 75.426 | −34.000 | 1.00 | 12.47 | A |
| ATOM | 5919 | CG | GLU | A | 753 | 44.140 | 75.575 | −32.822 | 1.00 | 14.62 | A |
| ATOM | 5920 | CD | GLU | A | 753 | 44.852 | 76.044 | −31.567 | 1.00 | 16.59 | A |
| ATOM | 5921 | OE1 | GLU | A | 753 | 45.201 | 77.241 | −31.489 | 1.00 | 19.53 | A |
| ATOM | 5922 | OE2 | GLU | A | 753 | 45.072 | 75.211 | −30.664 | 1.00 | 19.34 | A |
| ATOM | 5923 | C | GLU | A | 753 | 43.399 | 76.207 | −35.650 | 1.00 | 11.79 | A |
| ATOM | 5924 | O | GLU | A | 753 | 43.776 | 77.308 | −36.064 | 1.00 | 12.11 | A |
| ATOM | 5925 | N | SER | A | 754 | 42.124 | 75.896 | −35.453 | 1.00 | 10.70 | A |
| ATOM | 5926 | CA | SER | A | 754 | 41.050 | 76.850 | −35.675 | 1.00 | 10.81 | A |
| ATOM | 5927 | CE | SER | A | 754 | 40.227 | 76.458 | −36.901 | 1.00 | 11.07 | A |
| ATOM | 5928 | OG | SER | A | 754 | 40.988 | 76.604 | −38.086 | 1.00 | 10.80 | A |
| ATOM | 5929 | C | SER | A | 754 | 40.172 | 76.842 | −34.436 | 1.00 | 10.66 | A |
| ATOM | 5930 | O | SER | A | 754 | 40.221 | 75.902 | −33.638 | 1.00 | 10.36 | A |
| ATOM | 5931 | N | SER | A | 755 | 39.373 | 77.884 | −34.258 | 1.00 | 10.33 | A |
| ATOM | 5932 | CA | SER | A | 755 | 38.506 | 77.934 | −33.097 | 1.00 | 11.56 | A |
| ATOM | 5933 | CB | SER | A | 755 | 39.317 | 78.274 | −31.842 | 1.00 | 12.90 | A |
| ATOM | 5934 | OG | SER | A | 755 | 39.816 | 79.597 | −31.904 | 1.00 | 15.50 | A |
| ATOM | 5935 | C | SER | A | 755 | 37.378 | 78.934 | −33.233 | 1.00 | 10.74 | A |
| ATOM | 5936 | O | SER | A | 755 | 37.442 | 79.877 | −34.026 | 1.00 | 10.96 | A |
| ATOM | 5937 | N | VAL | A | 756 | 36.335 | 78.701 | −32.449 | 1.00 | 9.84 | A |
| ATOM | 5938 | CA | VAL | A | 756 | 35.177 | 79.574 | −32.397 | 1.00 | 10.44 | A |
| ATOM | 5939 | CB | VAL | A | 756 | 33.920 | 78.891 | −32.961 | 1.00 | 10.20 | A |
| ATOM | 5940 | CG1 | VAL | A | 756 | 32.716 | 79.801 | −32.774 | 1.00 | 10.96 | A |
| ATOM | 5941 | CG2 | VAL | A | 756 | 34.127 | 78.554 | −34.432 | 1.00 | 12.18 | A |
| ATOM | 5942 | C | VAL | A | 756 | 34.967 | 79.869 | −30.919 | 1.00 | 10.58 | A |
| ATOM | 5943 | O | VAL | A | 756 | 34.764 | 78.953 | −30.120 | 1.00 | 10.80 | A |
| ATOM | 5944 | N | SER | A | 757 | 35.041 | 81.143 | −30.552 | 1.00 | 10.27 | A |
| ATOM | 5945 | CA | SER | A | 757 | 34.864 | 81.546 | −29.164 | 1.00 | 10.99 | A |
| ATOM | 5946 | CB | SER | A | 757 | 36.148 | 82.187 | −28.631 | 1.00 | 11.96 | A |
| ATOM | 5947 | OG | SER | A | 757 | 37.258 | 81.326 | −28.808 | 1.00 | 14.85 | A |
| ATOM | 5948 | C | SER | A | 757 | 33.724 | 82.542 | −29.062 | 1.00 | 10.89 | A |
| ATOM | 5949 | O | SER | A | 757 | 33.579 | 83.411 | −29.917 | 1.00 | 11.64 | A |
| ATOM | 5950 | N | VAL | A | 758 | 32.918 | 82.423 | −28.015 | 1.00 | 10.48 | A |
| ATOM | 5951 | CA | VAL | A | 758 | 31.801 | 83.335 | −27.827 | 1.00 | 11.21 | A |
| ATOM | 5952 | CB | VAL | A | 758 | 30.471 | 82.678 | −28.285 | 1.00 | 11.67 | A |
| ATOM | 5953 | CG1 | VAL | A | 758 | 30.239 | 81.372 | −27.535 | 1.00 | 12.22 | A |
| ATOM | 5954 | CG2 | VAL | A | 758 | 29.315 | 83.636 | −28.067 | 1.00 | 11.35 | A |
| ATOM | 5955 | C | VAL | A | 758 | 31.691 | 83.790 | −26.376 | 1.00 | 11.49 | A |
| ATOM | 5956 | O | VAL | A | 758 | 31.858 | 82.995 | −25.446 | 1.00 | 11.17 | A |
| ATOM | 5957 | N | GLY | A | 759 | 31.429 | 85.081 | −26.196 | 1.00 | 11.26 | A |
| ATOM | 5958 | CA | GLY | A | 759 | 31.295 | 85.645 | −24.867 | 1.00 | 11.72 | A |
| ATOM | 5959 | C | GLY | A | 759 | 29.854 | 85.610 | −24.396 | 1.00 | 11.97 | A |
| ATOM | 5960 | O | GLY | A | 759 | 29.064 | 86.505 | −24.696 | 1.00 | 12.26 | A |
| ATOM | 5961 | N | LEU | A | 760 | 29.509 | 84.560 | −23.661 | 1.00 | 12.65 | A |
| ATOM | 5962 | CA | LEU | A | 760 | 28.160 | 84.388 | −23.139 | 1.00 | 13.29 | A |
| ATOM | 5963 | CB | LEU | A | 760 | 27.776 | 82.905 | −23.171 | 1.00 | 13.33 | A |
| ATOM | 5964 | CG | LEU | A | 760 | 27.892 | 82.180 | −24.513 | 1.00 | 13.38 | A |
| ATOM | 5965 | CD1 | LEU | A | 760 | 27.612 | 80.696 | −24.316 | 1.00 | 15.19 | A |
| ATOM | 5966 | CD2 | LEU | A | 760 | 26.920 | 82.785 | −25.518 | 1.00 | 14.79 | A |
| ATOM | 5967 | C | LEU | A | 760 | 28.107 | 84.883 | −21.701 | 1.00 | 13.87 | A |
| ATOM | 5968 | O | LEU | A | 760 | 29.143 | 85.085 | −21.068 | 1.00 | 14.41 | A |
| ATOM | 5969 | N | PRO | A | 761 | 26.897 | 85.102 | −21.168 | 1.00 | 14.84 | A |
| ATOM | 5970 | CD | PRO | A | 761 | 25.570 | 85.177 | −21.802 | 1.00 | 15.39 | A |
| ATOM | 5971 | CA | PRO | A | 761 | 26.844 | 85.569 | −19.781 | 1.00 | 14.68 | A |
| ATOM | 5972 | CB | PRO | A | 761 | 25.349 | 85.777 | −19.544 | 1.00 | 15.73 | A |
| ATOM | 5973 | CG | PRO | A | 761 | 24.850 | 86.157 | −20.905 | 1.00 | 15.81 | A |
| ATOM | 5974 | C | PRO | A | 761 | 27.429 | 84.489 | −18.867 | 1.00 | 14.74 | A |
| ATOM | 5975 | O | PRO | A | 761 | 26.973 | 83.343 | −18.879 | 1.00 | 15.36 | A |
| ATOM | 5976 | N | SER | A | 762 | 28.451 | 84.864 | −18.104 | 1.00 | 13.55 | A |
| ATOM | 5977 | CA | SER | A | 762 | 29.122 | 83.967 | −17.166 | 1.00 | 13.04 | A |
| ATOM | 5978 | CB | SER | A | 762 | 28.093 | 83.188 | −16.337 | 1.00 | 13.70 | A |
| ATOM | 5979 | OG | SER | A | 762 | 27.255 | 84.053 | −15.597 | 1.00 | 15.66 | A |
| ATOM | 5980 | C | SER | A | 762 | 30.080 | 82.961 | −17.794 | 1.00 | 12.29 | A |
| ATOM | 5981 | O | SER | A | 762 | 30.784 | 82.252 | −17.074 | 1.00 | 11.93 | A |
| ATOM | 5982 | N | VAL | A | 763 | 30.121 | 82.888 | −19.120 | 1.00 | 12.24 | A |
| ATOM | 5983 | CA | VAL | A | 763 | 30.993 | 81.911 | −19.760 | 1.00 | 11.70 | A |
| ATOM | 5984 | CB | VAL | A | 763 | 30.250 | 80.563 | −19.992 | 1.00 | 12.39 | A |
| ATOM | 5985 | CG1 | VAL | A | 763 | 31.211 | 79.528 | −20.572 | 1.00 | 12.05 | A |
| ATOM | 5986 | CG2 | VAL | A | 763 | 29.642 | 80.057 | −18.700 | 1.00 | 13.13 | A |
| ATOM | 5987 | C | VAL | A | 763 | 31.566 | 82.298 | −21.111 | 1.00 | 11.77 | A |
| ATOM | 5988 | O | VAL | A | 763 | 30.822 | 82.642 | −22.021 | 1.00 | 11.80 | A |
| ATOM | 5989 | N | VAL | A | 764 | 32.889 | 82.258 | −21.237 | 1.00 | 10.33 | A |
| ATOM | 5990 | CA | VAL | A | 764 | 33.497 | 82.488 | −22.536 | 1.00 | 10.30 | A |
| ATOM | 5991 | CB | VAL | A | 764 | 34.865 | 83.201 | −22.449 | 1.00 | 10.08 | A |
| ATOM | 5992 | CG1 | VAL | A | 764 | 35.489 | 83.291 | −23.844 | 1.00 | 11.18 | A |
| ATOM | 5993 | CG2 | VAL | A | 764 | 34.684 | 84.601 | −21.868 | 1.00 | 11.72 | A |
| ATOM | 5994 | C | VAL | A | 764 | 33.675 | 81.038 | −23.000 | 1.00 | 10.11 | A |
| ATOM | 5995 | O | VAL | A | 764 | 34.513 | 80.296 | −22.470 | 1.00 | 9.72 | A |
| ATOM | 5996 | N | HIS | A | 765 | 32.843 | 80.638 | −23.957 | 1.00 | 9.99 | A |
| ATOM | 5997 | CA | HIS | A | 765 | 32.814 | 79.281 | −24.502 | 1.00 | 9.87 | A |
| ATOM | 5998 | CB | HIS | A | 765 | 31.361 | 78.921 | −24.825 | 1.00 | 9.62 | A |
| ATOM | 5999 | CG | HIS | A | 765 | 31.172 | 77.533 | −25.348 | 1.00 | 9.93 | A |
| ATOM | 6000 | CD2 | HIS | A | 765 | 31.366 | 76.329 | −24.762 | 1.00 | 7.55 | A |
| ATOM | 6001 | ND1 | HIS | A | 765 | 30.686 | 77.271 | −26.611 | 1.00 | 11.37 | A |
| ATOM | 6002 | CE1 | HIS | A | 765 | 30.586 | 75.965 | −26.779 | 1.00 | 8.86 | A |
| ATOM | 6003 | NE2 | HIS | A | 765 | 30.992 | 75.370 | −25.671 | 1.00 | 11.67 | A |
| ATOM | 6004 | C | HIS | A | 765 | 33.684 | 79.178 | −25.750 | 1.00 | 10.43 | A |
| ATOM | 6005 | O | HIS | A | 765 | 33.546 | 79.975 | −26.674 | 1.00 | 9.54 | A |
| ATOM | 6006 | N | GLN | A | 766 | 34.569 | 78.189 | −25.787 | 1.00 | 9.10 | A |
| ATOM | 6007 | CA | GLN | A | 766 | 35.473 | 78.041 | −26.917 | 1.00 | 10.36 | A |
| ATOM | 6008 | CB | GLN | A | 766 | 36.886 | 78.461 | −26.503 | 1.00 | 11.74 | A |
| ATOM | 6009 | CG | GLN | A | 766 | 36.963 | 79.777 | −25.740 | 1.00 | 16.65 | A |
| ATOM | 6010 | CD | GLN | A | 766 | 38.093 | 79.787 | −24.720 | 1.00 | 18.99 | A |
| ATOM | 6011 | OE1 | GLN | A | 766 | 39.248 | 79.518 | −25.054 | 1.00 | 20.56 | A |
| ATOM | 6012 | NE2 | GLN | A | 766 | 37.761 | 80.098 | −23.465 | 1.00 | 19.49 | A |
| ATOM | 6013 | C | GLN | A | 766 | 35.534 | 76.617 | −27.450 | 1.00 | 9.67 | A |
| ATOM | 6014 | O | GLN | A | 766 | 35.657 | 75.664 | −26.679 | 1.00 | 9.31 | A |
| ATOM | 6015 | N | THR | A | 767 | 35.447 | 76.481 | −28.770 | 1.00 | 9.57 | A |
| ATOM | 6016 | CA | THR | A | 767 | 35.537 | 75.184 | −29.427 | 1.00 | 9.97 | A |
| ATOM | 6017 | CB | THR | A | 767 | 34.326 | 74.909 | −30.336 | 1.00 | 9.80 | A |
| ATOM | 6018 | OG1 | THR | A | 767 | 33.124 | 74.986 | −29.567 | 1.00 | 10.60 | A |
| ATOM | 6019 | CG2 | THR | A | 767 | 34.423 | 73.518 | −30.943 | 1.00 | 10.52 | A |
| ATOM | 6020 | C | THR | A | 767 | 36.797 | 75.248 | −30.277 | 1.00 | 10.72 | A |
| ATOM | 6021 | O | THR | A | 767 | 36.890 | 76.064 | −31.201 | 1.00 | 10.96 | A |
| ATOM | 6022 | N | ILE | A | 768 | 37.767 | 74.397 | −29.955 | 1.00 | 10.84 | A |
| ATOM | 6023 | CA | ILE | A | 768 | 39.044 | 74.374 | −30.659 | 1.00 | 11.39 | A |
| ATOM | 6024 | CB | ILE | A | 768 | 40.207 | 74.474 | −29.653 | 1.00 | 11.17 | A |
| ATOM | 6025 | CG2 | ILE | A | 768 | 41.536 | 74.614 | −30.391 | 1.00 | 12.50 | A |
| ATOM | 6026 | CG1 | ILE | A | 768 | 39.986 | 75.679 | −28.739 | 1.00 | 12.86 | A |
| ATOM | 6027 | CD1 | ILE | A | 768 | 40.930 | 75.727 | −27.552 | 1.00 | 14.09 | A |
| ATOM | 6028 | C | ILE | A | 768 | 39.210 | 73.114 | −31.499 | 1.00 | 11.59 | A |
| ATOM | 6029 | O | ILE | A | 768 | 38.924 | 72.006 | −31.044 | 1.00 | 11.70 | A |
| ATOM | 6030 | N | MSE | A | 769 | 39.679 | 73.295 | −32.729 | 1.00 | 11.90 | A |
| ATOM | 6031 | CA | MSE | A | 769 | 39.878 | 72.190 | −33.662 | 1.00 | 12.55 | A |
| ATOM | 6032 | CB | MSE | A | 769 | 39.004 | 72.403 | −34.898 | 1.00 | 15.34 | A |
| ATOM | 6033 | CG | MSE | A | 769 | 37.519 | 72.393 | −34.597 | 1.00 | 16.83 | A |
| ATOM | 6034 | SE | MSE | A | 769 | 36.503 | 73.347 | −35.920 | 1.00 | 26.54 | A |
| ATOM | 6035 | CE | MSE | A | 769 | 36.555 | 75.086 | −35.079 | 1.00 | 19.56 | A |
| ATOM | 6036 | C | MSE | A | 769 | 41.336 | 72.068 | −34.088 | 1.00 | 12.98 | A |
| ATOM | 6037 | O | MSE | A | 769 | 41.948 | 73.040 | −34.531 | 1.00 | 12.06 | A |
| ATOM | 6038 | N | ARG | A | 770 | 41.891 | 70.868 | −33.960 | 1.00 | 13.68 | A |
| ATOM | 6039 | CA | ARG | A | 770 | 43.275 | 70.640 | −34.339 | 1.00 | 15.07 | A |
| ATOM | 6040 | CB | ARG | A | 770 | 44.140 | 70.474 | −33.089 | 1.00 | 17.11 | A |
| ATOM | 6041 | CG | ARG | A | 770 | 44.074 | 71.679 | −32.165 | 1.00 | 20.18 | A |
| ATOM | 6042 | CD | ARG | A | 770 | 44.923 | 71.491 | −30.919 | 1.00 | 23.42 | A |
| ATOM | 6043 | NE | ARG | A | 770 | 44.600 | 72.493 | −29.908 | 1.00 | 26.56 | A |
| ATOM | 6044 | CZ | ARG | A | 770 | 45.160 | 72.556 | −28.705 | 1.00 | 27.92 | A |
| ATOM | 6045 | NH1 | ARG | A | 770 | 46.084 | 71.671 | −28.353 | 1.00 | 28.57 | A |
| ATOM | 6046 | NH2 | ARG | A | 770 | 44.790 | 73.500 | −27.850 | 1.00 | 28.79 | A |
| ATOM | 6047 | C | ARG | A | 770 | 43.435 | 69.434 | −35.253 | 1.00 | 15.19 | A |
| ATOM | 6048 | O | ARG | A | 770 | 44.541 | 68.934 | −35.442 | 1.00 | 15.98 | A |
| ATOM | 6049 | N | GLY | A | 771 | 42.325 | 68.968 | −35.814 | 1.00 | 15.00 | A |
| ATOM | 6050 | CA | GLY | A | 771 | 42.390 | 67.836 | −36.720 | 1.00 | 15.83 | A |
| ATOM | 6051 | C | GLY | A | 771 | 41.638 | 66.602 | −36.266 | 1.00 | 16.49 | A |
| ATOM | 6052 | O | GLY | A | 771 | 41.371 | 65.706 | −37.071 | 1.00 | 18.18 | A |
| ATOM | 6053 | N | GLY | A | 772 | 41.310 | 66.543 | −34.979 | 1.00 | 15.66 | A |
| ATOM | 6054 | CA | GLY | A | 772 | 40.581 | 65.404 | −34.446 | 1.00 | 15.30 | A |
| ATOM | 6055 | C | GLY | A | 772 | 39.374 | 65.872 | −33.659 | 1.00 | 13.91 | A |
| ATOM | 6056 | O | GLY | A | 772 | 38.767 | 66.886 | −33.999 | 1.00 | 13.73 | A |
| ATOM | 6057 | N | ALA | A | 773 | 39.012 | 65.135 | −32.613 | 1.00 | 13.00 | A |
| ATOM | 6058 | CA | ALA | A | 773 | 37.877 | 65.525 | −31.790 | 1.00 | 12.18 | A |
| ATOM | 6059 | CB | ALA | A | 773 | 37.699 | 64.549 | −30.642 | 1.00 | 12.99 | A |
| ATOM | 6060 | C | ALA | A | 773 | 38.162 | 66.921 | −31.249 | 1.00 | 11.57 | A |
| ATOM | 6061 | O | ALA | A | 773 | 39.277 | 67.216 | −30.819 | 1.00 | 12.06 | A |
| ATOM | 6062 | N | PRO | A | 774 | 37.161 | 67.804 | −31.273 | 1.00 | 11.20 | A |
| ATOM | 6063 | CD | PRO | A | 774 | 35.782 | 67.673 | −31.775 | 1.00 | 11.31 | A |
| ATOM | 6064 | CA | PRO | A | 774 | 37.409 | 69.152 | −30.761 | 1.00 | 10.53 | A |
| ATOM | 6065 | CB | PRO | A | 774 | 36.142 | 69.910 | −31.153 | 1.00 | 11.51 | A |
| ATOM | 6066 | CG | PRO | A | 774 | 35.089 | 68.847 | −31.110 | 1.00 | 12.74 | A |
| ATOM | 6067 | C | PRO | A | 774 | 37.660 | 69.219 | −29.262 | 1.00 | 10.06 | A |
| ATOM | 6068 | O | PRO | A | 774 | 37.322 | 68.300 | −28.507 | 1.00 | 9.96 | A |
| ATOM | 6069 | N | GLU | A | 775 | 38.287 | 70.311 | −28.849 | 1.00 | 9.33 | A |
| ATOM | 6070 | CA | GLU | A | 775 | 38.550 | 70.565 | −27.445 | 1.00 | 9.07 | A |
| ATOM | 6071 | CB | GLU | A | 775 | 39.992 | 71.021 | −27.213 | 1.00 | 9.95 | A |
| ATOM | 6072 | CG | GLU | A | 775 | 40.302 | 71.305 | −25.738 | 1.00 | 12.24 | A |
| ATOM | 6073 | CD | GLU | A | 775 | 41.714 | 71.809 | −25.499 | 1.00 | 15.25 | A |
| ATOM | 6074 | OE1 | GLU | A | 775 | 42.609 | 71.504 | −26.314 | 1.00 | 19.42 | A |
| ATOM | 6075 | OE2 | GLU | A | 775 | 41.934 | 72.497 | −24.478 | 1.00 | 16.73 | A |
| ATOM | 6076 | C | GLU | A | 775 | 37.608 | 71.701 | −27.096 | 1.00 | 9.08 | A |
| ATOM | 6077 | O | GLU | A | 775 | 37.431 | 72.632 | −27.886 | 1.00 | 9.71 | A |
| ATOM | 6078 | N | ILE | A | 776 | 36.988 | 71.620 | −25.931 | 1.00 | 9.14 | A |
| ATOM | 6079 | CA | ILE | A | 776 | 36.083 | 72.666 | −25.491 | 1.00 | 9.39 | A |
| ATOM | 6080 | CB | ILE | A | 776 | 34.696 | 72.103 | −25.114 | 1.00 | 10.30 | A |
| ATOM | 6081 | CG2 | ILE | A | 776 | 33.749 | 73.248 | −24.785 | 1.00 | 11.43 | A |
| ATOM | 6082 | CG1 | ILE | A | 776 | 34.154 | 71.219 | −26.240 | 1.00 | 12.19 | A |
| ATOM | 6083 | CD1 | ILE | A | 776 | 34.033 | 71.908 | −27.587 | 1.00 | 13.48 | A |
| ATOM | 6084 | C | ILE | A | 776 | 36.683 | 73.295 | −24.245 | 1.00 | 8.89 | A |
| ATOM | 6085 | O | ILE | A | 776 | 37.173 | 72.585 | −23.369 | 1.00 | 8.86 | A |
| ATOM | 6086 | N | ARG | A | 777 | 36.670 | 74.622 | −24.177 | 1.00 | 9.64 | A |
| ATOM | 6087 | CA | ARG | A | 777 | 37.178 | 75.329 | −23.007 | 1.00 | 9.16 | A |
| ATOM | 6088 | CB | ARG | A | 777 | 38.486 | 76.062 | −23.306 | 1.00 | 10.34 | A |
| ATOM | 6089 | CG | ARG | A | 777 | 39.647 | 75.161 | −23.655 | 1.00 | 10.26 | A |
| ATOM | 6090 | CD | ARG | A | 777 | 40.943 | 75.946 | −23.759 | 1.00 | 11.46 | A |
| ATOM | 6091 | NE | ARG | A | 777 | 42.013 | 75.116 | −24.308 | 1.00 | 12.09 | A |
| ATOM | 6092 | CZ | ARG | A | 777 | 43.198 | 75.575 | −24.697 | 1.00 | 13.87 | A |
| ATOM | 6093 | NH1 | ARG | A | 777 | 43.482 | 76.869 | −24.593 | 1.00 | 15.34 | A |
| ATOM | 6094 | NH2 | ARG | A | 777 | 44.090 | 74.744 | −25.221 | 1.00 | 15.77 | A |
| ATOM | 6095 | C | ARG | A | 777 | 36.139 | 76.346 | −22.589 | 1.00 | 8.98 | A |
| ATOM | 6096 | O | ARG | A | 777 | 35.619 | 77.093 | −23.423 | 1.00 | 9.21 | A |
| ATOM | 6097 | N | ASN | A | 778 | 35.821 | 76.355 | −21.303 | 1.00 | 8.45 | A |
| ATOM | 6098 | CA | ASN | A | 778 | 34.863 | 77.301 | −20.756 | 1.00 | 8.73 | A |
| ATOM | 6099 | CB | ASN | A | 778 | 33.672 | 76.597 | −20.098 | 1.00 | 9.56 | A |
| ATOM | 6100 | CG | ASN | A | 778 | 32.726 | 75.971 | −21.094 | 1.00 | 10.18 | A |
| ATOM | 6101 | OD1 | ASN | A | 778 | 32.682 | 76.360 | −22.261 | 1.00 | 10.34 | A |
| ATOM | 6102 | ND2 | ASN | A | 778 | 31.939 | 75.007 | −20.629 | 1.00 | 9.32 | A |
| ATOM | 6103 | C | ASN | A | 778 | 35.529 | 78.141 | −19.684 | 1.00 | 8.82 | A |
| ATOM | 6104 | O | ASN | A | 778 | 36.000 | 77.600 | −18.683 | 1.00 | 8.41 | A |
| ATOM | 6105 | N | LEU | A | 779 | 35.586 | 79.453 | −19.891 | 1.00 | 9.10 | A |
| ATOM | 6106 | CA | LEU | A | 779 | 36.133 | 80.338 | −18.868 | 1.00 | 9.56 | A |
| ATOM | 6107 | CB | LEU | A | 779 | 36.792 | 81.574 | −19.487 | 1.00 | 11.11 | A |
| ATOM | 6108 | CG | LEU | A | 779 | 37.363 | 82.583 | −18.484 | 1.00 | 12.38 | A |
| ATOM | 6109 | CD1 | LEU | A | 779 | 38.443 | 81.931 | −17.637 | 1.00 | 13.74 | A |
| ATOM | 6110 | CD2 | LEU | A | 779 | 37.924 | 83.779 | −19.242 | 1.00 | 14.30 | A |
| ATOM | 6111 | C | LEU | A | 779 | 34.869 | 80.723 | −18.112 | 1.00 | 9.41 | A |
| ATOM | 6112 | O | LEU | A | 779 | 34.092 | 81.567 | −18.563 | 1.00 | 10.31 | A |
| ATOM | 6113 | N | VAL | A | 780 | 34.659 | 80.083 | −16.969 | 1.00 | 8.89 | A |
| ATOM | 6114 | CA | VAL | A | 780 | 33.457 | 80.297 | −16.178 | 1.00 | 9.72 | A |
| ATOM | 6115 | CB | VAL | A | 780 | 32.948 | 78.948 | −15.610 | 1.00 | 9.29 | A |
| ATOM | 6116 | CG1 | VAL | A | 780 | 31.607 | 79.142 | −14.917 | 1.00 | 10.76 | A |
| ATOM | 6117 | CG2 | VAL | A | 780 | 32.827 | 77.927 | −16.735 | 1.00 | 9.53 | A |
| ATOM | 6118 | C | VAL | A | 780 | 33.607 | 81.290 | −15.036 | 1.00 | 10.33 | A |
| ATOM | 6119 | O | VAL | A | 780 | 34.404 | 81.090 | −14.123 | 1.00 | 10.64 | A |
| ATOM | 6120 | N | ASP | A | 781 | 32.832 | 82.368 | −15.105 | 1.00 | 11.16 | A |
| ATOM | 6121 | CA | ASP | A | 781 | 32.838 | 83.401 | −14.076 | 1.00 | 11.93 | A |
| ATOM | 6122 | CB | ASP | A | 781 | 33.522 | 84.670 | −14.583 | 1.00 | 14.09 | A |
| ATOM | 6123 | CG | ASP | A | 781 | 33.596 | 85.750 | −13.522 | 1.00 | 15.28 | A |
| ATOM | 6124 | OD1 | ASP | A | 781 | 34.106 | 86.850 | −13.823 | 1.00 | 18.04 | A |
| ATOM | 6125 | OD2 | ASP | A | 781 | 33.146 | 85.499 | −12.384 | 1.00 | 15.28 | A |
| ATOM | 6126 | C | ASP | A | 781 | 31.384 | 83.693 | −13.748 | 1.00 | 12.36 | A |
| ATOM | 6127 | O | ASP | A | 781 | 30.751 | 84.548 | −14.372 | 1.00 | 11.82 | A |
| ATOM | 6128 | N | ILE | A | 782 | 30.861 | 82.962 | −12.770 | 1.00 | 13.05 | A |
| ATOM | 6129 | CA | ILE | A | 782 | 29.474 | 83.088 | −12.348 | 1.00 | 15.17 | A |
| ATOM | 6130 | CB | ILE | A | 782 | 29.110 | 81.918 | −11.399 | 1.00 | 15.24 | A |
| ATOM | 6131 | CG2 | ILE | A | 782 | 29.747 | 82.136 | −10.032 | 1.00 | 15.58 | A |
| ATOM | 6132 | CG1 | ILE | A | 782 | 27.595 | 81.776 | −11.289 | 1.00 | 15.55 | A |
| ATOM | 6133 | CD1 | ILE | A | 782 | 27.163 | 80.458 | −10.671 | 1.00 | 15.33 | A |
| ATOM | 6134 | C | ILE | A | 782 | 29.202 | 84.437 | −11.680 | 1.00 | 16.86 | A |
| ATOM | 6135 | O | ILE | A | 782 | 28.061 | 84.759 | −11.341 | 1.00 | 18.15 | A |
| ATOM | 6136 | N | GLY | A | 783 | 30.260 | 85.224 | −11.503 | 1.00 | 17.28 | A |
| ATOM | 6137 | CA | GLY | A | 783 | 30.126 | 86.545 | −10.908 | 1.00 | 19.48 | A |
| ATOM | 6138 | C | GLY | A | 783 | 29.290 | 86.621 | −9.646 | 1.00 | 20.51 | A |
| ATOM | 6139 | O | GLY | A | 783 | 29.538 | 85.895 | −8.682 | 1.00 | 21.41 | A |
| ATOM | 6140 | N | SER | A | 784 | 28.293 | 87.503 | −9.649 | 1.00 | 22.21 | A |
| ATOM | 6141 | CA | SER | A | 784 | 27.435 | 87.675 | −8.483 | 1.00 | 23.63 | A |
| ATOM | 6142 | CB | SER | A | 784 | 27.325 | 89.160 | −8.128 | 1.00 | 24.57 | A |
| ATOM | 6143 | OG | SER | A | 784 | 26.727 | 89.896 | −9.180 | 1.00 | 26.76 | A |
| ATOM | 6144 | C | SER | A | 784 | 26.035 | 87.086 | −8.649 | 1.00 | 24.17 | A |
| ATOM | 6145 | O | SER | A | 784 | 25.109 | 87.470 | −7.934 | 1.00 | 24.52 | A |
| ATOM | 6146 | N | LEU | A | 785 | 25.880 | 86.158 | −9.589 | 1.00 | 24.17 | A |
| ATOM | 6147 | CA | LEU | A | 785 | 24.585 | 85.520 | −9.819 | 1.00 | 24.30 | A |
| ATOM | 6148 | CB | LEU | A | 785 | 24.589 | 84.756 | −11.147 | 1.00 | 25.03 | A |
| ATOM | 6149 | CG | LEU | A | 785 | 24.586 | 85.570 | −12.444 | 1.00 | 26.37 | A |
| ATOM | 6150 | CD1 | LEU | A | 785 | 25.792 | 86.490 | −12.492 | 1.00 | 27.91 | A |
| ATOM | 6151 | CD2 | LEU | A | 785 | 24.597 | 84.623 | −13.631 | 1.00 | 27.18 | A |
| ATOM | 6152 | C | LEU | A | 785 | 24.282 | 84.554 | −8.678 | 1.00 | 24.04 | A |
| ATOM | 6153 | O | LEU | A | 785 | 24.590 | 83.364 | −8.760 | 1.00 | 24.23 | A |
| ATOM | 6154 | N | ASP | A | 786 | 23.666 | 85.068 | −7.620 | 1.00 | 23.21 | A |
| ATOM | 6155 | CA | ASP | A | 786 | 23.345 | 84.247 | −6.460 | 1.00 | 22.19 | A |
| ATOM | 6156 | CB | ASP | A | 786 | 22.877 | 85.130 | −5.302 | 1.00 | 24.01 | A |
| ATOM | 6157 | CG | ASP | A | 786 | 23.851 | 86.245 | −4.993 | 1.00 | 25.65 | A |
| ATOM | 6158 | OD1 | ASP | A | 786 | 25.048 | 85.955 | −4.776 | 1.00 | 24.91 | A |
| ATOM | 6159 | OD2 | ASP | A | 786 | 23.418 | 87.417 | −4.968 | 1.00 | 26.93 | A |
| ATOM | 6160 | C | ASP | A | 786 | 22.289 | 83.188 | −6.747 | 1.00 | 20.42 | A |
| ATOM | 6161 | O | ASP | A | 786 | 21.423 | 83.368 | −7.604 | 1.00 | 20.14 | A |
| ATOM | 6162 | N | ASN | A | 787 | 22.378 | 82.085 | −6.012 | 1.00 | 18.59 | A |
| ATOM | 6163 | CA | ASN | A | 787 | 21.450 | 80.971 | −6.145 | 1.00 | 17.29 | A |
| ATOM | 6164 | CB | ASN | A | 787 | 20.104 | 81.341 | −5.522 | 1.00 | 18.36 | A |
| ATOM | 6165 | CG | ASN | A | 787 | 20.230 | 81.678 | −4.054 | 1.00 | 19.72 | A |
| ATOM | 6166 | OD1 | ASN | A | 787 | 20.851 | 80.936 | −3.292 | 1.00 | 20.47 | A |
| ATOM | 6167 | ND2 | ASN | A | 787 | 19.644 | 82.799 | −3.646 | 1.00 | 22.13 | A |
| ATOM | 6168 | C | ASN | A | 787 | 21.267 | 80.536 | −7.590 | 1.00 | 16.22 | A |
| ATOM | 6169 | O | ASN | A | 787 | 20.145 | 80.387 | −8.077 | 1.00 | 15.49 | A |
| ATOM | 6170 | N | THR | A | 788 | 22.389 | 80.321 | −8.266 | 1.00 | 14.90 | A |
| ATOM | 6171 | CA | THR | A | 788 | 22.382 | 79.901 | −9.656 | 1.00 | 13.74 | A |
| ATOM | 6172 | CB | THR | A | 788 | 22.718 | 81.081 | −10.594 | 1.00 | 15.15 | A |
| ATOM | 6173 | OG1 | THR | A | 788 | 21.782 | 82.145 | −10.380 | 1.00 | 15.54 | A |
| ATOM | 6174 | CG2 | THR | A | 788 | 22.649 | 80.646 | −12.053 | 1.00 | 14.91 | A |
| ATOM | 6175 | C | THR | A | 788 | 23.427 | 78.817 | −9.874 | 1.00 | 12.17 | A |
| ATOM | 6176 | O | THR | A | 788 | 24.516 | 78.872 | −9.306 | 1.00 | 12.24 | A |
| ATOM | 6177 | N | GLU | A | 789 | 23.075 | 77.823 | −10.679 | 1.00 | 11.16 | A |
| ATOM | 6178 | CA | GLU | A | 789 | 23.993 | 76.750 | −11.021 | 1.00 | 10.70 | A |
| ATOM | 6179 | CB | GLU | A | 789 | 23.523 | 75.417 | −10.417 | 1.00 | 10.65 | A |
| ATOM | 6180 | CG | GLU | A | 789 | 23.410 | 75.468 | −8.893 | 1.00 | 11.01 | A |
| ATOM | 6181 | CD | GLU | A | 789 | 23.467 | 74.103 | −8.230 | 1.00 | 10.75 | A |
| ATOM | 6182 | OE1 | GLU | A | 789 | 24.244 | 73.242 | −8.700 | 1.00 | 11.43 | A |
| ATOM | 6183 | OE2 | GLU | A | 789 | 22.751 | 73.900 | −7.224 | 1.00 | 11.36 | A |
| ATOM | 6184 | C | GLU | A | 789 | 23.977 | 76.717 | −12.545 | 1.00 | 10.53 | A |
| ATOM | 6185 | O | GLU | A | 789 | 22.925 | 76.541 | −13.159 | 1.00 | 12.21 | A |
| ATOM | 6186 | N | ILE | A | 790 | 25.141 | 76.929 | −13.151 | 1.00 | 9.89 | A |
| ATOM | 6187 | CA | ILE | A | 790 | 25.258 | 76.951 | −14.603 | 1.00 | 11.05 | A |
| ATOM | 6188 | CB | ILE | A | 790 | 26.356 | 77.928 | −15.063 | 1.00 | 11.53 | A |
| ATOM | 6189 | CG2 | ILE | A | 790 | 26.444 | 77.932 | −16.584 | 1.00 | 11.91 | A |
| ATOM | 6190 | CG1 | ILE | A | 790 | 26.048 | 79.335 | −14.545 | 1.00 | 14.02 | A |
| ATOM | 6191 | CD1 | ILE | A | 790 | 27.164 | 80.331 | −14.786 | 1.00 | 16.91 | A |
| ATOM | 6192 | C | ILE | A | 790 | 25.601 | 75.577 | −15.143 | 1.00 | 10.36 | A |
| ATOM | 6193 | O | ILE | A | 790 | 26.590 | 74.967 | −14.735 | 1.00 | 10.51 | A |
| ATOM | 6194 | N | VAL | A | 791 | 24.783 | 75.096 | −16.070 | 1.00 | 10.03 | A |
| ATOM | 6195 | CA | VAL | A | 791 | 25.003 | 73.790 | −16.664 | 1.00 | 10.13 | A |
| ATOM | 6196 | CB | VAL | A | 791 | 23.792 | 72.848 | −16.409 | 1.00 | 10.46 | A |
| ATOM | 6197 | CG1 | VAL | A | 791 | 22.582 | 73.304 | −17.224 | 1.00 | 11.49 | A |
| ATOM | 6198 | CG2 | VAL | A | 791 | 24.163 | 71.414 | −16.760 | 1.00 | 11.24 | A |
| ATOM | 6199 | C | VAL | A | 791 | 25.239 | 73.873 | −18.166 | 1.00 | 9.93 | A |
| ATOM | 6200 | O | VAL | A | 791 | 24.653 | 74.714 | −18.860 | 1.00 | 10.16 | A |
| ATOM | 6201 | N | MSE | A | 792 | 26.128 | 73.022 | −18.662 | 1.00 | 9.51 | A |
| ATOM | 6202 | CA | MSE | A | 792 | 26.394 | 72.954 | −20.092 | 1.00 | 9.67 | A |
| ATOM | 6203 | CB | MSE | A | 792 | 27.892 | 72.921 | −20.392 | 1.00 | 11.43 | A |
| ATOM | 6204 | CG | MSE | A | 792 | 28.185 | 72.769 | −21.881 | 1.00 | 12.60 | A |
| ATOM | 6205 | SE | MSE | A | 792 | 30.059 | 72.856 | −22.319 | 1.00 | 18.80 | A |
| ATOM | 6206 | CE | MSE | A | 792 | 30.632 | 71.215 | −21.499 | 1.00 | 14.20 | A |
| ATOM | 6207 | C | MSE | A | 792 | 25.749 | 71.657 | −20.559 | 1.00 | 9.89 | A |
| ATOM | 6208 | O | MSE | A | 792 | 26.063 | 70.581 | −20.046 | 1.00 | 10.12 | A |
| ATOM | 6209 | N | ARG | A | 793 | 24.843 | 71.760 | −21.524 | 1.00 | 9.88 | A |
| ATOM | 6210 | CA | ARG | A | 793 | 24.143 | 70.589 | −22.027 | 1.00 | 9.40 | A |
| ATOM | 6211 | CB | ARG | A | 793 | 22.636 | 70.756 | −21.786 | 1.00 | 9.08 | A |
| ATOM | 6212 | CG | ARG | A | 793 | 21.775 | 69.579 | −22.249 | 1.00 | 9.24 | A |
| ATOM | 6213 | CD | ARG | A | 793 | 20.300 | 69.824 | −21.940 | 1.00 | 10.28 | A |
| ATOM | 6214 | NE | ARG | A | 793 | 20.051 | 69.905 | −20.504 | 1.00 | 11.04 | A |
| ATOM | 6215 | CZ | ARG | A | 793 | 18.958 | 70.427 | −19.959 | 1.00 | 10.72 | A |
| ATOM | 6216 | NH1 | ARG | A | 793 | 17.995 | 70.922 | −20.733 | 1.00 | 12.36 | A |
| ATOM | 6217 | NH2 | ARG | A | 793 | 18.832 | 70.473 | −18.640 | 1.00 | 11.35 | A |
| ATOM | 6218 | C | ARG | A | 793 | 24.396 | 70.333 | −23.503 | 1.00 | 9.92 | A |
| ATOM | 6219 | O | ARG | A | 793 | 24.593 | 71.264 | −24.286 | 1.00 | 10.28 | A |
| ATOM | 6220 | N | LEU | A | 794 | 24.393 | 69.055 | −23.866 | 1.00 | 10.51 | A |
| ATOM | 6221 | CA | LEU | A | 794 | 24.563 | 68.618 | −25.248 | 1.00 | 10.31 | A |
| ATOM | 6222 | CB | LEU | A | 794 | 25.730 | 67.633 | −25.370 | 1.00 | 11.42 | A |
| ATOM | 6223 | CG | LEU | A | 794 | 27.129 | 68.240 | −25.492 | 1.00 | 11.51 | A |
| ATOM | 6224 | CD1 | LEU | A | 794 | 28.180 | 67.207 | −25.131 | 1.00 | 12.30 | A |
| ATOM | 6225 | CD2 | LEU | A | 794 | 27.336 | 68.748 | −26.914 | 1.00 | 13.24 | A |
| ATOM | 6226 | C | LEU | A | 794 | 23.265 | 67.921 | −25.643 | 1.00 | 11.62 | A |
| ATOM | 6227 | O | LEU | A | 794 | 22.789 | 67.038 | −24.931 | 1.00 | 10.91 | A |
| ATOM | 6228 | N | GLU | A | 795 | 22.686 | 68.330 | −26.766 | 1.00 | 11.83 | A |
| ATOM | 6229 | CA | GLU | A | 795 | 21.444 | 67.731 | −27.244 | 1.00 | 13.21 | A |
| ATOM | 6230 | CB | GLU | A | 795 | 20.373 | 68.814 | −27.427 | 1.00 | 13.79 | A |
| ATOM | 6231 | CG | GLU | A | 795 | 20.156 | 69.665 | −26.184 | 1.00 | 16.95 | A |
| ATOM | 6232 | CD | GLU | A | 795 | 19.134 | 70.774 | −26.382 | 1.00 | 18.99 | A |
| ATOM | 6233 | OE1 | GLU | A | 795 | 19.138 | 71.404 | −27.460 | 1.00 | 21.87 | A |
| ATOM | 6234 | OE2 | GLU | A | 795 | 18.339 | 71.029 | −25.452 | 1.00 | 20.56 | A |
| ATOM | 6235 | C | GLU | A | 795 | 21.716 | 67.018 | −28.565 | 1.00 | 13.30 | A |
| ATOM | 6236 | O | GLU | A | 795 | 22.259 | 67.609 | −29.500 | 1.00 | 13.97 | A |
| ATOM | 6237 | N | THR | A | 796 | 21.352 | 65.741 | −28.637 | 1.00 | 12.67 | A |
| ATOM | 6238 | CA | THR | A | 796 | 21.577 | 64.957 | −29.846 | 1.00 | 13.15 | A |
| ATOM | 6239 | CB | THR | A | 796 | 22.771 | 63.991 | −29.688 | 1.00 | 12.88 | A |
| ATOM | 6240 | OG1 | THR | A | 796 | 22.372 | 62.859 | −28.900 | 1.00 | 11.73 | A |
| ATOM | 6241 | CG2 | THR | A | 796 | 23.935 | 64.687 | −29.002 | 1.00 | 13.25 | A |
| ATOM | 6242 | C | THR | A | 796 | 20.369 | 64.101 | −30.203 | 1.00 | 13.86 | A |
| ATOM | 6243 | O | THR | A | 796 | 19.355 | 64.120 | −29.509 | 1.00 | 16.03 | A |
| ATOM | 6244 | N | HIS | A | 797 | 20.511 | 63.341 | −31.284 | 1.00 | 15.53 | A |
| ATOM | 6245 | CA | HIS | A | 797 | 19.465 | 62.444 | −31.764 | 1.00 | 16.70 | A |
| ATOM | 6246 | CB | HIS | A | 797 | 19.343 | 62.542 | −33.283 | 1.00 | 18.40 | A |
| ATOM | 6247 | CG | HIS | A | 797 | 18.639 | 63.775 | −33.747 | 1.00 | 18.87 | A |
| ATOM | 6248 | CD2 | HIS | A | 797 | 17.877 | 64.669 | −33.075 | 1.00 | 18.85 | A |
| ATOM | 6249 | ND1 | HIS | A | 797 | 18.669 | 64.204 | −35.056 | 1.00 | 19.59 | A |
| ATOM | 6250 | CE1 | HIS | A | 797 | 17.956 | 65.310 | −35.169 | 1.00 | 19.23 | A |
| ATOM | 6251 | NE2 | HIS | A | 797 | 17.465 | 65.613 | −33.981 | 1.00 | 18.80 | A |
| ATOM | 6252 | C | HIS | A | 797 | 19.762 | 61.006 | −31.381 | 1.00 | 17.12 | A |
| ATOM | 6253 | O | HIS | A | 797 | 19.058 | 60.087 | −31.799 | 1.00 | 18.02 | A |
| ATOM | 6254 | N | ILE | A | 798 | 20.814 | 60.809 | −30.595 | 1.00 | 15.17 | A |
| ATOM | 6255 | CA | ILE | A | 798 | 21.190 | 59.473 | −30.158 | 1.00 | 14.07 | A |
| ATOM | 6256 | CB | ILE | A | 798 | 22.492 | 59.525 | −29.330 | 1.00 | 13.19 | A |
| ATOM | 6257 | CG2 | ILE | A | 798 | 22.845 | 58.132 | −28.802 | 1.00 | 12.33 | A |
| ATOM | 6258 | CG1 | ILE | A | 798 | 23.623 | 60.070 | −30.207 | 1.00 | 13.23 | A |
| ATOM | 6259 | CD1 | ILE | A | 798 | 24.934 | 60.303 | −29.468 | 1.00 | 13.56 | A |
| ATOM | 6260 | C | ILE | A | 798 | 20.047 | 58.887 | −29.331 | 1.00 | 13.73 | A |
| ATOM | 6261 | O | ILE | A | 798 | 19.554 | 59.518 | −28.399 | 1.00 | 13.69 | A |
| ATOM | 6262 | N | ASP | A | 799 | 19.617 | 57.681 | −29.697 | 1.00 | 14.35 | A |
| ATOM | 6263 | CA | ASP | A | 799 | 18.519 | 57.009 | −29.013 | 1.00 | 14.00 | A |
| ATOM | 6264 | CB | ASP | A | 799 | 17.849 | 56.018 | −29.970 | 1.00 | 15.90 | A |
| ATOM | 6265 | CG | ASP | A | 799 | 16.539 | 55.485 | −29.436 | 1.00 | 17.22 | A |
| ATOM | 6266 | OD1 | ASP | A | 799 | 16.007 | 54.522 | −30.025 | 1.00 | 18.99 | A |
| ATOM | 6267 | OD2 | ASP | A | 799 | 16.036 | 56.028 | −28.434 | 1.00 | 19.56 | A |
| ATOM | 6268 | C | ASP | A | 799 | 19.031 | 56.270 | −27.780 | 1.00 | 13.90 | A |
| ATOM | 6269 | O | ASP | A | 799 | 18.965 | 55.045 | −27.701 | 1.00 | 13.45 | A |
| ATOM | 6270 | N | SER | A | 800 | 19.529 | 57.031 | −26.812 | 1.00 | 13.23 | A |
| ATOM | 6271 | CA | SER | A | 800 | 20.077 | 56.462 | −25.587 | 1.00 | 12.57 | A |
| ATOM | 6272 | CB | SER | A | 800 | 21.039 | 57.466 | −24.943 | 1.00 | 12.35 | A |
| ATOM | 6273 | OG | SER | A | 800 | 20.372 | 58.671 | −24.618 | 1.00 | 11.94 | A |
| ATOM | 6274 | C | SER | A | 800 | 19.021 | 56.038 | −24.570 | 1.00 | 11.90 | A |
| ATOM | 6275 | O | SER | A | 800 | 19.303 | 55.237 | −23.679 | 1.00 | 11.76 | A |
| ATOM | 6276 | N | GLY | A | 801 | 17.812 | 56.576 | −24.694 | 1.00 | 11.77 | A |
| ATOM | 6277 | CA | GLY | A | 801 | 16.751 | 56.225 | −23.766 | 1.00 | 11.64 | A |
| ATOM | 6278 | C | GLY | A | 801 | 16.994 | 56.750 | −22.367 | 1.00 | 11.57 | A |
| ATOM | 6279 | O | GLY | A | 801 | 17.063 | 57.956 | −22.157 | 1.00 | 12.52 | A |
| ATOM | 6280 | N | ASP | A | 802 | 17.118 | 55.840 | −21.406 | 1.00 | 11.43 | A |
| ATOM | 6281 | CA | ASP | A | 802 | 17.358 | 56.225 | −20.021 | 1.00 | 11.22 | A |
| ATOM | 6282 | CB | ASP | A | 802 | 16.274 | 55.636 | −19.107 | 1.00 | 13.10 | A |
| ATOM | 6283 | CG | ASP | A | 802 | 16.173 | 54.118 | −19.201 | 1.00 | 13.45 | A |
| ATOM | 6284 | OD1 | ASP | A | 802 | 15.264 | 53.554 | −18.553 | 1.00 | 15.07 | A |
| ATOM | 6285 | OD2 | ASP | A | 802 | 16.990 | 53.486 | −19.908 | 1.00 | 15.03 | A |
| ATOM | 6286 | C | ASP | A | 802 | 18.736 | 55.771 | −19.556 | 1.00 | 10.91 | A |
| ATOM | 6287 | O | ASP | A | 802 | 19.041 | 55.807 | −18.362 | 1.00 | 10.76 | A |
| ATOM | 6288 | N | ILE | A | 803 | 19.571 | 55.370 | −20.507 | 1.00 | 9.82 | A |
| ATOM | 6289 | CA | ILE | A | 803 | 20.910 | 54.888 | −20.197 | 1.00 | 9.93 | A |
| ATOM | 6290 | CB | ILE | A | 803 | 21.208 | 53.572 | −20.958 | 1.00 | 9.82 | A |
| ATOM | 6291 | CG2 | ILE | A | 803 | 22.634 | 53.103 | −20.670 | 1.00 | 9.69 | A |
| ATOM | 6292 | CG1 | ILE | A | 803 | 20.191 | 52.496 | −20.564 | 1.00 | 9.60 | A |
| ATOM | 6293 | CD1 | ILE | A | 803 | 20.226 | 52.125 | −19.093 | 1.00 | 10.76 | A |
| ATOM | 6294 | C | ILE | A | 803 | 22.020 | 55.872 | −20.537 | 1.00 | 9.64 | A |
| ATOM | 6295 | O | ILE | A | 803 | 21.969 | 56.562 | −21.556 | 1.00 | 9.87 | A |
| ATOM | 6296 | N | PHE | A | 804 | 23.020 | 55.940 | −19.662 | 1.00 | 9.22 | A |
| ATOM | 6297 | CA | PHE | A | 804 | 24.188 | 56.776 | −19.889 | 1.00 | 8.73 | A |
| ATOM | 6298 | CB | PHE | A | 804 | 23.913 | 58.265 | −19.583 | 1.00 | 8.57 | A |
| ATOM | 6299 | CG | PHE | A | 804 | 23.561 | 58.572 | −18.151 | 1.00 | 8.50 | A |
| ATOM | 6300 | CD1 | PHE | A | 804 | 24.496 | 59.178 | −17.311 | 1.00 | 8.09 | A |
| ATOM | 6301 | CD2 | PHE | A | 804 | 22.272 | 58.351 | −17.667 | 1.00 | 8.51 | A |
| ATOM | 6302 | CE1 | PHE | A | 804 | 24.152 | 59.568 | −16.016 | 1.00 | 8.03 | A |
| ATOM | 6303 | CE2 | PHE | A | 804 | 21.917 | 58.738 | −16.370 | 1.00 | 9.55 | A |
| ATOM | 6304 | CZ | PHE | A | 804 | 22.862 | 59.351 | −15.546 | 1.00 | 9.47 | A |
| ATOM | 6305 | C | PHE | A | 804 | 25.324 | 56.208 | −19.059 | 1.00 | 8.03 | A |
| ATOM | 6306 | O | PHE | A | 804 | 25.100 | 55.363 | −18.192 | 1.00 | 8.86 | A |
| ATOM | 6307 | N | TYR | A | 805 | 26.543 | 56.636 | −19.346 | 1.00 | 7.73 | A |
| ATOM | 6308 | CA | TYR | A | 805 | 27.696 | 56.117 | −18.635 | 1.00 | 7.16 | A |
| ATOM | 6309 | CB | TYR | A | 805 | 28.558 | 55.279 | −19.582 | 1.00 | 7.36 | A |
| ATOM | 6310 | CG | TYR | A | 805 | 27.858 | 54.059 | −20.133 | 1.00 | 8.65 | A |
| ATOM | 6311 | CD1 | TYR | A | 805 | 26.873 | 54.174 | −21.117 | 1.00 | 9.16 | A |
| ATOM | 6312 | CE1 | TYR | A | 805 | 26.232 | 53.047 | −21.626 | 1.00 | 8.73 | A |
| ATOM | 6313 | CD2 | TYR | A | 805 | 28.184 | 52.786 | −19.669 | 1.00 | 8.73 | A |
| ATOM | 6314 | CE2 | TYR | A | 805 | 27.547 | 51.651 | −20.171 | 1.00 | 9.56 | A |
| ATOM | 6315 | CZ | TYR | A | 805 | 26.576 | 51.792 | −21.149 | 1.00 | 10.03 | A |
| ATOM | 6316 | OH | TYR | A | 805 | 25.956 | 50.674 | −21.664 | 1.00 | 10.71 | A |
| ATOM | 6317 | C | TYR | A | 805 | 28.536 | 57.232 | −18.052 | 1.00 | 7.37 | A |
| ATOM | 6318 | O | TYR | A | 805 | 28.700 | 58.275 | −18.675 | 1.00 | 8.06 | A |
| ATOM | 6319 | N | THR | A | 806 | 29.046 | 57.011 | −16.844 | 1.00 | 7.62 | A |
| ATOM | 6320 | CA | THR | A | 806 | 29.908 | 57.985 | −16.187 | 1.00 | 7.14 | A |
| ATOM | 6321 | CB | THR | A | 806 | 29.179 | 58.741 | −15.060 | 1.00 | 7.06 | A |
| ATOM | 6322 | OG1 | THR | A | 806 | 28.822 | 57.829 | −14.016 | 1.00 | 8.45 | A |
| ATOM | 6323 | CG2 | THR | A | 806 | 27.922 | 59.398 | −15.586 | 1.00 | 7.42 | A |
| ATOM | 6324 | C | THR | A | 806 | 31.062 | 57.188 | −15.599 | 1.00 | 7.29 | A |
| ATOM | 6325 | O | THR | A | 806 | 30.936 | 55.986 | −15.368 | 1.00 | 7.37 | A |
| ATOM | 6326 | N | ASP | A | 807 | 32.195 | 57.834 | −15.366 | 1.00 | 7.07 | A |
| ATOM | 6327 | CA | ASP | A | 807 | 33.319 | 57.096 | −14.820 | 1.00 | 7.59 | A |
| ATOM | 6328 | CB | ASP | A | 807 | 34.632 | 57.567 | −15.444 | 1.00 | 8.85 | A |
| ATOM | 6329 | CG | ASP | A | 807 | 35.082 | 58.908 | −14.912 | 1.00 | 9.02 | A |
| ATOM | 6330 | OD1 | ASP | A | 807 | 36.215 | 58.983 | −14.389 | 1.00 | 10.87 | A |
| ATOM | 6331 | OD2 | ASP | A | 807 | 34.310 | 59.883 | −15.013 | 1.00 | 9.58 | A |
| ATOM | 6332 | C | ASP | A | 807 | 33.418 | 57.207 | −13.313 | 1.00 | 7.31 | A |
| ATOM | 6333 | O | ASP | A | 807 | 32.801 | 58.069 | −12.686 | 1.00 | 7.29 | A |
| ATOM | 6334 | N | LEU | A | 808 | 34.193 | 56.299 | −12.742 | 1.00 | 7.26 | A |
| ATOM | 6335 | CA | LEU | A | 808 | 34.435 | 56.291 | −11.315 | 1.00 | 6.64 | A |
| ATOM | 6336 | CB | LEU | A | 808 | 33.996 | 54.960 | −10.697 | 1.00 | 7.52 | A |
| ATOM | 6337 | CG | LEU | A | 808 | 32.480 | 54.762 | −10.576 | 1.00 | 7.74 | A |
| ATOM | 6338 | CD1 | LEU | A | 808 | 32.171 | 53.301 | −10.300 | 1.00 | 9.05 | A |
| ATOM | 6339 | CD2 | LEU | A | 808 | 31.927 | 55.646 | −9.466 | 1.00 | 8.74 | A |
| ATOM | 6340 | C | LEU | A | 808 | 35.931 | 56.493 | −11.120 | 1.00 | 7.13 | A |
| ATOM | 6341 | O | LEU | A | 808 | 36.740 | 55.662 | −11.537 | 1.00 | 6.82 | A |
| ATOM | 6342 | N | ASN | A | 809 | 36.284 | 57.635 | −10.536 | 1.00 | 6.18 | A |
| ATOM | 6343 | CA | ASN | A | 809 | 37.671 | 57.976 | −10.229 | 1.00 | 6.39 | A |
| ATOM | 6344 | CB | ASN | A | 809 | 38.141 | 57.084 | −9.077 | 1.00 | 5.55 | A |
| ATOM | 6345 | CG | ASN | A | 809 | 37.154 | 57.061 | −7.935 | 1.00 | 6.45 | A |
| ATOM | 6346 | OD1 | ASN | A | 809 | 37.176 | 57.931 | −7.051 | 1.00 | 8.57 | A |
| ATOM | 6347 | ND2 | ASN | A | 809 | 36.259 | 56.083 | −7.957 | 1.00 | 4.53 | A |
| ATOM | 6348 | C | ASN | A | 809 | 38.643 | 57.871 | −11.401 | 1.00 | 6.62 | A |
| ATOM | 6349 | O | ASN | A | 809 | 39.830 | 57.619 | −11.202 | 1.00 | 6.76 | A |
| ATOM | 6350 | N | GLY | A | 810 | 38.144 | 58.072 | −12.618 | 1.00 | 6.22 | A |
| ATOM | 6351 | CA | GLY | A | 810 | 38.998 | 57.994 | −13.795 | 1.00 | 7.72 | A |
| ATOM | 6352 | C | GLY | A | 810 | 39.568 | 56.606 | −14.038 | 1.00 | 8.56 | A |
| ATOM | 6353 | O | GLY | A | 810 | 40.536 | 56.437 | −14.788 | 1.00 | 9.54 | A |
| ATOM | 6354 | N | LEU | A | 811 | 38.950 | 55.606 | −13.420 | 1.00 | 7.78 | A |
| ATOM | 6355 | CA | LEU | A | 811 | 39.410 | 54.225 | −13.532 | 1.00 | 8.21 | A |
| ATOM | 6356 | CB | LEU | A | 811 | 39.541 | 53.622 | −12.127 | 1.00 | 8.72 | A |
| ATOM | 6357 | CG | LEU | A | 811 | 39.955 | 52.154 | −12.012 | 1.00 | 10.15 | A |
| ATOM | 6358 | CD1 | LEU | A | 811 | 41.366 | 51.988 | −12.539 | 1.00 | 11.19 | A |
| ATOM | 6359 | CD2 | LEU | A | 811 | 39.874 | 51.707 | −10.559 | 1.00 | 10.32 | A |
| ATOM | 6360 | C | LEU | A | 811 | 38.520 | 53.312 | −14.370 | 1.00 | 9.02 | A |
| ATOM | 6361 | O | LEU | A | 811 | 39.015 | 52.459 | −15.108 | 1.00 | 10.92 | A |
| ATOM | 6362 | N | GLN | A | 812 | 37.212 | 53.501 | −14.262 | 1.00 | 8.03 | A |
| ATOM | 6363 | CA | GLN | A | 812 | 36.264 | 52.644 | −14.962 | 1.00 | 8.29 | A |
| ATOM | 6364 | CB | GLN | A | 812 | 35.907 | 51.464 | −14.056 | 1.00 | 9.25 | A |
| ATOM | 6365 | CG | GLN | A | 812 | 35.335 | 51.928 | −12.710 | 1.00 | 10.20 | A |
| ATOM | 6366 | CD | GLN | A | 812 | 35.023 | 50.790 | −11.756 | 1.00 | 12.79 | A |
| ATOM | 6367 | OE1 | GLN | A | 812 | 34.052 | 50.058 | −11.938 | 1.00 | 13.30 | A |
| ATOM | 6368 | NE2 | GLN | A | 812 | 35.851 | 50.640 | −10.727 | 1.00 | 13.55 | A |
| ATOM | 6369 | C | GLN | A | 812 | 34.992 | 53.398 | −15.298 | 1.00 | 8.35 | A |
| ATOM | 6370 | O | GLN | A | 812 | 34.680 | 54.415 | −14.671 | 1.00 | 7.86 | A |
| ATOM | 6371 | N | PHE | A | 813 | 34.260 | 52.903 | −16.291 | 1.00 | 7.68 | A |
| ATOM | 6372 | CA | PHE | A | 813 | 32.993 | 53.515 | −16.656 | 1.00 | 7.67 | A |
| ATOM | 6373 | CB | PHE | A | 813 | 32.926 | 53.810 | −18.155 | 1.00 | 7.05 | A |
| ATOM | 6374 | CG | PHE | A | 813 | 33.686 | 55.041 | −18.546 | 1.00 | 7.39 | A |
| ATOM | 6375 | CD1 | PHE | A | 813 | 35.072 | 55.012 | −18.646 | 1.00 | 7.39 | A |
| ATOM | 6376 | CD2 | PHE | A | 813 | 33.019 | 56.247 | −18.741 | 1.00 | 8.36 | A |
| ATOM | 6377 | CE1 | PHE | A | 813 | 35.788 | 56.170 | −18.932 | 1.00 | 9.02 | A |
| ATOM | 6378 | CE2 | PHE | A | 813 | 33.725 | 57.410 | −19.027 | 1.00 | 8.21 | A |
| ATOM | 6379 | CZ | PHE | A | 813 | 35.115 | 57.368 | −19.121 | 1.00 | 8.05 | A |
| ATOM | 6380 | C | PHE | A | 813 | 31.876 | 52.583 | −16.240 | 1.00 | 7.95 | A |
| ATOM | 6381 | O | PHE | A | 813 | 31.884 | 51.387 | −16.561 | 1.00 | 8.99 | A |
| ATOM | 6382 | N | ILE | A | 814 | 30.921 | 53.140 | −15.509 | 1.00 | 7.80 | A |
| ATOM | 6383 | CA | ILE | A | 814 | 29.799 | 52.379 | −14.990 | 1.00 | 8.05 | A |
| ATOM | 6384 | CB | ILE | A | 814 | 29.720 | 52.552 | −13.450 | 1.00 | 7.86 | A |
| ATOM | 6385 | CG2 | ILE | A | 814 | 29.397 | 53.998 | −13.102 | 1.00 | 8.17 | A |
| ATOM | 6386 | CG1 | ILE | A | 814 | 28.688 | 51.616 | −12.851 | 1.00 | 8.21 | A |
| ATOM | 6387 | CD1 | ILE | A | 814 | 28.763 | 51.486 | −11.329 | 1.00 | 8.23 | A |
| ATOM | 6388 | C | ILE | A | 814 | 28.490 | 52.812 | −15.646 | 1.00 | 7.18 | A |
| ATOM | 6389 | O | ILE | A | 814 | 28.262 | 53.997 | −15.900 | 1.00 | 6.96 | A |
| ATOM | 6390 | N | LYS | A | 815 | 27.641 | 51.834 | −15.933 | 1.00 | 7.64 | A |
| ATOM | 6391 | CA | LYS | A | 815 | 26.355 | 52.097 | −16.557 | 1.00 | 8.17 | A |
| ATOM | 6392 | CB | LYS | A | 815 | 25.764 | 50.787 | −17.078 | 1.00 | 9.06 | A |
| ATOM | 6393 | CG | LYS | A | 815 | 24.457 | 50.909 | −17.845 | 1.00 | 11.44 | A |
| ATOM | 6394 | CD | LYS | A | 815 | 24.060 | 49.539 | −18.389 | 1.00 | 13.34 | A |
| ATOM | 6395 | CE | LYS | A | 815 | 22.850 | 49.609 | −19.297 | 1.00 | 16.25 | A |
| ATOM | 6396 | NZ | LYS | A | 815 | 22.538 | 48.269 | −19.879 | 1.00 | 18.48 | A |
| ATOM | 6397 | C | LYS | A | 815 | 25.392 | 52.744 | −15.572 | 1.00 | 7.90 | A |
| ATOM | 6398 | O | LYS | A | 815 | 25.240 | 52.288 | −14.437 | 1.00 | 7.72 | A |
| ATOM | 6399 | N | ARG | A | 816 | 24.752 | 53.817 | −16.016 | 1.00 | 7.58 | A |
| ATOM | 6400 | CA | ARG | A | 816 | 23.779 | 54.533 | −15.205 | 1.00 | 7.54 | A |
| ATOM | 6401 | CB | ARG | A | 816 | 24.133 | 56.023 | −15.103 | 1.00 | 8.01 | A |
| ATOM | 6402 | CG | ARG | A | 816 | 25.524 | 56.341 | −14.580 | 1.00 | 8.73 | A |
| ATOM | 6403 | CD | ARG | A | 816 | 25.718 | 55.751 | −13.203 | 1.00 | 9.16 | A |
| ATOM | 6404 | NE | ARG | A | 816 | 26.879 | 56.317 | −12.522 | 1.00 | 8.38 | A |
| ATOM | 6405 | CZ | ARG | A | 816 | 27.259 | 55.952 | −11.304 | 1.00 | 8.64 | A |
| ATOM | 6406 | NH1 | ARG | A | 816 | 26.567 | 55.027 | −10.654 | 1.00 | 7.99 | A |
| ATOM | 6407 | NH2 | ARG | A | 816 | 28.311 | 56.519 | −10.728 | 1.00 | 8.64 | A |
| ATOM | 6408 | C | ARG | A | 816 | 22.419 | 54.436 | −15.874 | 1.00 | 8.06 | A |
| ATOM | 6409 | O | ARG | A | 816 | 22.327 | 54.376 | −17.099 | 1.00 | 7.94 | A |
| ATOM | 6410 | N | ARG | A | 817 | 21.366 | 54.419 | −15.066 | 1.00 | 8.58 | A |
| ATOM | 6411 | CA | ARG | A | 817 | 20.016 | 54.403 | −15.606 | 1.00 | 9.41 | A |
| ATOM | 6412 | CB | ARG | A | 817 | 19.302 | 53.073 | −15.330 | 1.00 | 9.62 | A |
| ATOM | 6413 | CG | ARG | A | 817 | 17.830 | 53.098 | −15.749 | 1.00 | 11.25 | A |
| ATOM | 6414 | CD | ARG | A | 817 | 17.123 | 51.758 | −15.571 | 1.00 | 13.10 | A |
| ATOM | 6415 | NE | ARG | A | 817 | 17.649 | 50.728 | −16.460 | 1.00 | 13.49 | A |
| ATOM | 6416 | CZ | ARG | A | 817 | 18.408 | 49.713 | −16.062 | 1.00 | 14.00 | A |
| ATOM | 6417 | NH1 | ARG | A | 817 | 18.736 | 49.588 | −14.781 | 1.00 | 13.99 | A |
| ATOM | 6418 | NH2 | ARG | A | 817 | 18.838 | 48.820 | −16.942 | 1.00 | 15.05 | A |
| ATOM | 6419 | C | ARG | A | 817 | 19.249 | 55.539 | −14.953 | 1.00 | 9.86 | A |
| ATOM | 6420 | O | ARG | A | 817 | 19.118 | 55.586 | −13.731 | 1.00 | 10.49 | A |
| ATOM | 6421 | N | ARG | A | 818 | 18.780 | 56.475 | −15.772 | 1.00 | 9.41 | A |
| ATOM | 6422 | CA | ARG | A | 818 | 18.003 | 57.598 | −15.276 | 1.00 | 11.11 | A |
| ATOM | 6423 | CB | ARG | A | 818 | 17.653 | 58.548 | −16.422 | 1.00 | 12.00 | A |
| ATOM | 6424 | CG | ARG | A | 818 | 16.996 | 59.843 | −15.967 | 1.00 | 12.11 | A |
| ATOM | 6425 | CD | ARG | A | 818 | 16.321 | 60.572 | −17.123 | 1.00 | 13.88 | A |
| ATOM | 6426 | NE | ARG | A | 818 | 15.045 | 59.954 | −17.470 | 1.00 | 16.57 | A |
| ATOM | 6427 | CZ | ARG | A | 818 | 14.776 | 59.370 | −18.634 | 1.00 | 15.90 | A |
| ATOM | 6428 | NH1 | ARG | A | 818 | 15.694 | 59.317 | −19.591 | 1.00 | 15.20 | A |
| ATOM | 6429 | NH2 | ARG | A | 818 | 13.580 | 58.832 | −18.837 | 1.00 | 17.09 | A |
| ATOM | 6430 | C | ARG | A | 818 | 16.723 | 57.005 | −14.703 | 1.00 | 11.22 | A |
| ATOM | 6431 | O | ARG | A | 818 | 16.058 | 56.209 | −15.366 | 1.00 | 12.29 | A |
| ATOM | 6432 | N | LEU | A | 819 | 16.382 | 57.384 | −13.477 | 1.00 | 11.61 | A |
| ATOM | 6433 | CA | LEU | A | 819 | 15.179 | 56.873 | −12.829 | 1.00 | 12.56 | A |
| ATOM | 6434 | CB | LEU | A | 819 | 15.545 | 56.177 | −11.517 | 1.00 | 13.02 | A |
| ATOM | 6435 | CG | LEU | A | 819 | 16.481 | 54.969 | −11.645 | 1.00 | 13.25 | A |
| ATOM | 6436 | CD1 | LEU | A | 819 | 16.901 | 54.493 | −10.260 | 1.00 | 13.84 | A |
| ATOM | 6437 | CD2 | LEU | A | 819 | 15.787 | 53.856 | −12.420 | 1.00 | 14.25 | A |
| ATOM | 6438 | C | LEU | A | 819 | 14.210 | 58.013 | −12.554 | 1.00 | 13.08 | A |
| ATOM | 6439 | O | LEU | A | 819 | 14.459 | 58.857 | −11.696 | 1.00 | 12.85 | A |
| ATOM | 6440 | N | ASP | A | 820 | 13.101 | 58.037 | −13.281 | 1.00 | 14.34 | A |
| ATOM | 6441 | CA | ASP | A | 820 | 12.130 | 59.097 | −13.088 | 1.00 | 14.92 | A |
| ATOM | 6442 | CB | ASP | A | 820 | 11.147 | 59.133 | −14.261 | 1.00 | 16.01 | A |
| ATOM | 6443 | CG | ASP | A | 820 | 11.841 | 59.387 | −15.586 | 1.00 | 17.37 | A |
| ATOM | 6444 | OD1 | ASP | A | 820 | 12.852 | 60.123 | −15.596 | 1.00 | 17.51 | A |
| ATOM | 6445 | OD2 | ASP | A | 820 | 11.378 | 58.862 | −16.619 | 1.00 | 19.76 | A |
| ATOM | 6446 | C | ASP | A | 820 | 11.397 | 58.964 | −11.757 | 1.00 | 14.77 | A |
| ATOM | 6447 | O | ASP | A | 820 | 10.706 | 59.888 | −11.329 | 1.00 | 15.17 | A |
| ATOM | 6448 | N | LYS | A | 821 | 11.559 | 57.824 | −11.089 | 1.00 | 14.77 | A |
| ATOM | 6449 | CA | LYS | A | 821 | 10.913 | 57.630 | −9.795 | 1.00 | 14.66 | A |
| ATOM | 6450 | CB | LYS | A | 821 | 10.826 | 56.138 | −9.445 | 1.00 | 14.60 | A |
| ATOM | 6451 | CG | LYS | A | 821 | 12.163 | 55.452 | −9.211 | 1.00 | 14.35 | A |
| ATOM | 6452 | CD | LYS | A | 821 | 11.997 | 53.942 | −9.154 | 1.00 | 14.29 | A |
| ATOM | 6453 | CE | LYS | A | 821 | 13.335 | 53.242 | −8.952 | 1.00 | 14.37 | A |
| ATOM | 6454 | NZ | LYS | A | 821 | 13.215 | 51.764 | −9.110 | 1.00 | 13.74 | A |
| ATOM | 6455 | C | LYS | A | 821 | 11.699 | 58.384 | −8.723 | 1.00 | 14.86 | A |
| ATOM | 6456 | O | LYS | A | 821 | 11.250 | 58.520 | −7.586 | 1.00 | 15.44 | A |
| ATOM | 6457 | N | LEU | A | 822 | 12.873 | 58.882 | −9.102 | 1.00 | 14.33 | A |
| ATOM | 6458 | CA | LEU | A | 822 | 13.718 | 59.644 | −8.188 | 1.00 | 13.96 | A |
| ATOM | 6459 | CB | LEU | A | 822 | 15.136 | 59.067 | −8.157 | 1.00 | 13.85 | A |
| ATOM | 6460 | CG | LEU | A | 822 | 15.292 | 57.607 | −7.720 | 1.00 | 14.60 | A |
| ATOM | 6461 | CD1 | LEU | A | 822 | 16.770 | 57.238 | −7.716 | 1.00 | 14.62 | A |
| ATOM | 6462 | CD2 | LEU | A | 822 | 14.682 | 57.401 | −6.338 | 1.00 | 14.91 | A |
| ATOM | 6463 | C | LEU | A | 822 | 13.771 | 61.091 | −8.666 | 1.00 | 14.06 | A |
| ATOM | 6464 | O | LEU | A | 822 | 13.600 | 61.362 | −9.854 | 1.00 | 14.20 | A |
| ATOM | 6465 | N | PRO | A | 823 | 14.007 | 62.040 | −7.747 | 1.00 | 13.37 | A |
| ATOM | 6466 | CD | PRO | A | 823 | 14.170 | 61.894 | −6.290 | 1.00 | 13.31 | A |
| ATOM | 6467 | CA | PRO | A | 823 | 14.073 | 63.450 | −8.146 | 1.00 | 13.08 | A |
| ATOM | 6468 | CB | PRO | A | 823 | 14.109 | 64.191 | −6.810 | 1.00 | 13.38 | A |
| ATOM | 6469 | CG | PRO | A | 823 | 14.794 | 63.219 | −5.902 | 1.00 | 14.39 | A |
| ATOM | 6470 | C | PRO | A | 823 | 15.287 | 63.743 | −9.026 | 1.00 | 12.91 | A |
| ATOM | 6471 | O | PRO | A | 823 | 16.253 | 62.982 | −9.050 | 1.00 | 12.74 | A |
| ATOM | 6472 | N | LEU | A | 824 | 15.222 | 64.855 | −9.749 | 1.00 | 12.02 | A |
| ATOM | 6473 | CA | LEU | A | 824 | 16.284 | 65.262 | −10.658 | 1.00 | 11.44 | A |
| ATOM | 6474 | CB | LEU | A | 824 | 16.019 | 66.702 | −11.119 | 1.00 | 11.62 | A |
| ATOM | 6475 | CG | LEU | A | 824 | 16.802 | 67.283 | −12.297 | 1.00 | 11.24 | A |
| ATOM | 6476 | CD1 | LEU | A | 824 | 16.058 | 68.500 | −12.841 | 1.00 | 11.66 | A |
| ATOM | 6477 | CD2 | LEU | A | 824 | 18.217 | 67.649 | −11.863 | 1.00 | 11.13 | A |
| ATOM | 6478 | C | LEU | A | 824 | 17.692 | 65.146 | −10.066 | 1.00 | 11.55 | A |
| ATOM | 6479 | O | LEU | A | 824 | 18.583 | 64.559 | −10.684 | 1.00 | 11.18 | A |
| ATOM | 6480 | N | GLN | A | 825 | 17.884 | 65.694 | −8.870 | 1.00 | 11.18 | A |
| ATOM | 6481 | CA | GLN | A | 825 | 19.197 | 65.675 | −8.226 | 1.00 | 10.79 | A |
| ATOM | 6482 | CB | GLN | A | 825 | 19.175 | 66.502 | −6.931 | 1.00 | 11.13 | A |
| ATOM | 6483 | CG | GLN | A | 825 | 18.246 | 65.972 | −5.846 | 1.00 | 10.84 | A |
| ATOM | 6484 | CD | GLN | A | 825 | 16.829 | 66.517 | −5.952 | 1.00 | 11.30 | A |
| ATOM | 6485 | OE1 | GLN | A | 825 | 16.400 | 66.980 | −7.011 | 1.00 | 12.37 | A |
| ATOM | 6486 | NE2 | GLN | A | 825 | 16.092 | 66.450 | −4.850 | 1.00 | 11.42 | A |
| ATOM | 6487 | C | GLN | A | 825 | 19.741 | 64.280 | −7.934 | 1.00 | 11.24 | A |
| ATOM | 6488 | O | GLN | A | 825 | 20.953 | 64.104 | −7.778 | 1.00 | 11.09 | A |
| ATOM | 6489 | N | ALA | A | 826 | 18.855 | 63.291 | −7.866 | 1.00 | 10.37 | A |
| ATOM | 6490 | CA | ALA | A | 826 | 19.278 | 61.921 | −7.597 | 1.00 | 10.73 | A |
| ATOM | 6491 | CB | ALA | A | 826 | 18.088 | 61.085 | −7.131 | 1.00 | 10.59 | A |
| ATOM | 6492 | C | ALA | A | 826 | 19.884 | 61.304 | −8.851 | 1.00 | 10.42 | A |
| ATOM | 6493 | O | ALA | A | 826 | 20.657 | 60.348 | −8.776 | 1.00 | 11.05 | A |
| ATOM | 6494 | N | ASN | A | 827 | 19.532 | 61.857 | −10.008 | 1.00 | 9.96 | A |
| ATOM | 6495 | CA | ASN | A | 827 | 20.039 | 61.349 | −11.272 | 1.00 | 9.71 | A |
| ATOM | 6496 | CB | ASN | A | 827 | 18.964 | 61.489 | −12.349 | 1.00 | 10.42 | A |
| ATOM | 6497 | CG | ASN | A | 827 | 17.836 | 60.490 | −12.157 | 1.00 | 11.62 | A |
| ATOM | 6498 | OD1 | ASN | A | 827 | 18.050 | 59.282 | −12.241 | 1.00 | 11.81 | A |
| ATOM | 6499 | ND2 | ASN | A | 827 | 16.639 | 60.986 | −11.879 | 1.00 | 12.88 | A |
| ATOM | 6500 | C | ASN | A | 827 | 21.343 | 62.005 | −11.694 | 1.00 | 9.32 | A |
| ATOM | 6501 | O | ASN | A | 827 | 21.849 | 61.765 | −12.788 | 1.00 | 9.65 | A |
| ATOM | 6502 | N | TYR | A | 828 | 21.883 | 62.834 | −10.807 | 1.00 | 9.16 | A |
| ATOM | 6503 | CA | TYR | A | 828 | 23.160 | 63.484 | −11.056 | 1.00 | 8.54 | A |
| ATOM | 6504 | CB | TYR | A | 828 | 23.222 | 64.852 | −10.378 | 1.00 | 8.84 | A |
| ATOM | 6505 | CG | TYR | A | 828 | 23.250 | 65.996 | −11.363 | 1.00 | 9.15 | A |
| ATOM | 6506 | CD1 | TYR | A | 828 | 22.189 | 66.211 | −12.243 | 1.00 | 8.96 | A |
| ATOM | 6507 | CE1 | TYR | A | 828 | 22.235 | 67.235 | −13.187 | 1.00 | 9.56 | A |
| ATOM | 6508 | CD2 | TYR | A | 828 | 24.357 | 66.839 | −11.447 | 1.00 | 9.13 | A |
| ATOM | 6509 | CE2 | TYR | A | 828 | 24.412 | 67.863 | −12.384 | 1.00 | 10.20 | A |
| ATOM | 6510 | CZ | TYR | A | 828 | 23.351 | 68.055 | −13.252 | 1.00 | 9.60 | A |
| ATOM | 6511 | OH | TYR | A | 828 | 23.418 | 69.048 | −14.198 | 1.00 | 10.53 | A |
| ATOM | 6512 | C | TYR | A | 828 | 24.224 | 62.573 | −10.459 | 1.00 | 8.13 | A |
| ATOM | 6513 | O | TYR | A | 828 | 24.041 | 62.030 | −9.370 | 1.00 | 9.32 | A |
| ATOM | 6514 | N | TYR | A | 829 | 25.324 | 62.398 | −11.182 | 1.00 | 7.92 | A |
| ATOM | 6515 | CA | TYR | A | 829 | 26.414 | 61.550 | −10.726 | 1.00 | 7.93 | A |
| ATOM | 6516 | CB | TYR | A | 829 | 26.458 | 60.251 | −11.533 | 1.00 | 7.94 | A |
| ATOM | 6517 | CG | TYR | A | 829 | 25.293 | 59.338 | −11.271 | 1.00 | 8.32 | A |
| ATOM | 6518 | CD1 | TYR | A | 829 | 24.122 | 59.432 | −12.023 | 1.00 | 8.62 | A |
| ATOM | 6519 | CE1 | TYR | A | 829 | 23.028 | 58.613 | −11.752 | 1.00 | 8.83 | A |
| ATOM | 6520 | CD2 | TYR | A | 829 | 25.346 | 58.402 | −10.241 | 1.00 | 7.20 | A |
| ATOM | 6521 | CE2 | TYR | A | 829 | 24.258 | 57.579 | −9.958 | 1.00 | 8.66 | A |
| ATOM | 6522 | CZ | TYR | A | 829 | 23.103 | 57.690 | −10.717 | 1.00 | 8.09 | A |
| ATOM | 6523 | OH | TYR | A | 829 | 22.023 | 56.884 | −10.434 | 1.00 | 9.63 | A |
| ATOM | 6524 | C | TYR | A | 829 | 27.745 | 62.257 | −10.887 | 1.00 | 7.92 | A |
| ATOM | 6525 | O | TYR | A | 829 | 27.848 | 63.264 | −11.584 | 1.00 | 8.32 | A |
| ATOM | 6526 | N | PRO | A | 830 | 28.787 | 61.740 | −10.228 | 1.00 | 7.99 | A |
| ATOM | 6527 | CD | PRO | A | 830 | 28.853 | 60.610 | −9.286 | 1.00 | 9.08 | A |
| ATOM | 6528 | CA | PRO | A | 830 | 30.085 | 62.395 | −10.376 | 1.00 | 8.33 | A |
| ATOM | 6529 | CB | PRO | A | 830 | 30.971 | 61.672 | −9.355 | 1.00 | 10.29 | A |
| ATOM | 6530 | CG | PRO | A | 830 | 30.008 | 60.995 | −8.414 | 1.00 | 10.63 | A |
| ATOM | 6531 | C | PRO | A | 830 | 30.588 | 62.134 | −11.797 | 1.00 | 7.87 | A |
| ATOM | 6532 | O | PRO | A | 830 | 30.367 | 61.050 | −12.345 | 1.00 | 7.95 | A |
| ATOM | 6533 | N | ILE | A | 831 | 31.225 | 63.128 | −12.406 | 1.00 | 7.13 | A |
| ATOM | 6534 | CA | ILE | A | 831 | 31.816 | 62.946 | −13.728 | 1.00 | 7.22 | A |
| ATOM | 6535 | CB | ILE | A | 831 | 31.253 | 63.925 | −14.791 | 1.00 | 7.38 | A |
| ATOM | 6536 | CG2 | ILE | A | 831 | 31.722 | 63.484 | −16.179 | 1.00 | 7.54 | A |
| ATOM | 6537 | CG1 | ILE | A | 831 | 29.715 | 63.962 | −14.744 | 1.00 | 7.69 | A |
| ATOM | 6538 | CD1 | ILE | A | 831 | 29.019 | 62.634 | −15.056 | 1.00 | 7.75 | A |
| ATOM | 6539 | C | ILE | A | 831 | 33.285 | 63.274 | −13.468 | 1.00 | 7.77 | A |
| ATOM | 6540 | O | ILE | A | 831 | 33.774 | 64.351 | −13.821 | 1.00 | 7.24 | A |
| ATOM | 6541 | N | PRO | A | 832 | 34.007 | 62.353 | −12.810 | 1.00 | 7.31 | A |
| ATOM | 6542 | CD | PRO | A | 832 | 33.562 | 61.108 | −12.159 | 1.00 | 7.92 | A |
| ATOM | 6543 | CA | PRO | A | 832 | 35.417 | 62.626 | −12.525 | 1.00 | 8.14 | A |
| ATOM | 6544 | CB | PRO | A | 832 | 35.821 | 61.469 | −11.600 | 1.00 | 7.96 | A |
| ATOM | 6545 | CG | PRO | A | 832 | 34.862 | 60.381 | −11.944 | 1.00 | 8.53 | A |
| ATOM | 6546 | C | PRO | A | 832 | 36.322 | 62.793 | −13.739 | 1.00 | 8.42 | A |
| ATOM | 6547 | O | PRO | A | 832 | 37.307 | 63.523 | −13.665 | 1.00 | 10.36 | A |
| ATOM | 6548 | N | SER | A | 833 | 35.996 | 62.142 | −14.853 | 1.00 | 7.99 | A |
| ATOM | 6549 | CA | SER | A | 833 | 36.822 | 62.293 | −16.049 | 1.00 | 7.57 | A |
| ATOM | 6550 | CB | SER | A | 833 | 38.028 | 61.346 | −16.004 | 1.00 | 9.46 | A |
| ATOM | 6551 | OG | SER | A | 833 | 37.664 | 60.014 | −16.297 | 1.00 | 11.27 | A |
| ATOM | 6552 | C | SER | A | 833 | 36.082 | 62.103 | −17.370 | 1.00 | 7.78 | A |
| ATOM | 6553 | O | SER | A | 833 | 36.607 | 62.443 | −18.426 | 1.00 | 6.86 | A |
| ATOM | 6554 | N | GLY | A | 834 | 34.868 | 61.570 | −17.328 | 1.00 | 6.96 | A |
| ATOM | 6555 | CA | GLY | A | 834 | 34.156 | 61.391 | −18.582 | 1.00 | 7.42 | A |
| ATOM | 6556 | C | GLY | A | 834 | 32.773 | 60.792 | −18.479 | 1.00 | 6.62 | A |
| ATOM | 6557 | O | GLY | A | 834 | 32.394 | 60.209 | −17.462 | 1.00 | 8.20 | A |
| ATOM | 6558 | N | MSE | A | 835 | 32.012 | 60.942 | −19.556 | 1.00 | 6.65 | A |
| ATOM | 6559 | CA | MSE | A | 835 | 30.658 | 60.420 | −19.609 | 1.00 | 7.41 | A |
| ATOM | 6560 | CB | MSE | A | 835 | 29.691 | 61.418 | −18.964 | 1.00 | 9.50 | A |
| ATOM | 6561 | CG | MSE | A | 835 | 29.580 | 62.740 | −19.725 | 1.00 | 10.76 | A |
| ATOM | 6562 | SE | MSE | A | 835 | 28.541 | 64.091 | −18.811 | 1.00 | 18.06 | A |
| ATOM | 6563 | CE | MSE | A | 835 | 26.954 | 63.069 | −18.482 | 1.00 | 14.51 | A |
| ATOM | 6564 | C | MSE | A | 835 | 30.281 | 60.220 | −21.071 | 1.00 | 7.29 | A |
| ATOM | 6565 | O | MSE | A | 835 | 30.833 | 60.874 | −21.960 | 1.00 | 7.55 | A |
| ATOM | 6566 | N | PHE | A | 836 | 29.358 | 59.302 | −21.329 | 1.00 | 7.61 | A |
| ATOM | 6567 | CA | PHE | A | 836 | 28.914 | 59.098 | −22.697 | 1.00 | 8.24 | A |
| ATOM | 6568 | CB | PHE | A | 836 | 29.939 | 58.263 | −23.501 | 1.00 | 7.78 | A |
| ATOM | 6569 | CG | PHE | A | 836 | 30.090 | 56.824 | −23.057 | 1.00 | 8.69 | A |
| ATOM | 6570 | CD1 | PHE | A | 836 | 29.230 | 55.834 | −23.536 | 1.00 | 8.19 | A |
| ATOM | 6571 | CD2 | PHE | A | 836 | 31.148 | 56.444 | −22.232 | 1.00 | 9.29 | A |
| ATOM | 6572 | CE1 | PHE | A | 836 | 29.428 | 54.489 | −23.205 | 1.00 | 10.52 | A |
| ATOM | 6573 | CE2 | PHE | A | 836 | 31.353 | 55.100 | −21.896 | 1.00 | 9.66 | A |
| ATOM | 6574 | CZ | PHE | A | 836 | 30.490 | 54.122 | −22.388 | 1.00 | 9.92 | A |
| ATOM | 6575 | C | PHE | A | 836 | 27.529 | 58.493 | −22.795 | 1.00 | 9.14 | A |
| ATOM | 6576 | O | PHE | A | 836 | 27.011 | 57.927 | −21.832 | 1.00 | 8.90 | A |
| ATOM | 6577 | N | ILE | A | 837 | 26.912 | 58.686 | −23.956 | 1.00 | 9.21 | A |
| ATOM | 6578 | CA | ILE | A | 837 | 25.604 | 58.120 | −24.257 | 1.00 | 9.71 | A |
| ATOM | 6579 | CB | ILE | A | 837 | 24.474 | 59.175 | −24.341 | 1.00 | 9.37 | A |
| ATOM | 6580 | CG2 | ILE | A | 837 | 24.046 | 59.587 | −22.947 | 1.00 | 10.54 | A |
| ATOM | 6581 | CG1 | ILE | A | 837 | 24.907 | 60.357 | −25.208 | 1.00 | 9.45 | A |
| ATOM | 6582 | CD1 | ILE | A | 837 | 23.765 | 61.309 | −25.552 | 1.00 | 9.78 | A |
| ATOM | 6583 | C | ILE | A | 837 | 25.766 | 57.468 | −25.616 | 1.00 | 9.49 | A |
| ATOM | 6584 | O | ILE | A | 837 | 26.618 | 57.878 | −26.413 | 1.00 | 9.37 | A |
| ATOM | 6585 | N | GLU | A | 838 | 24.968 | 56.442 | −25.881 | 1.00 | 9.39 | A |
| ATOM | 6586 | CA | GLU | A | 838 | 25.068 | 55.748 | −27.155 | 1.00 | 10.00 | A |
| ATOM | 6587 | CB | GLU | A | 838 | 26.232 | 54.751 | −27.114 | 1.00 | 10.07 | A |
| ATOM | 6588 | CG | GLU | A | 838 | 25.991 | 53.609 | −26.121 | 1.00 | 11.82 | A |
| ATOM | 6589 | CD | GLU | A | 838 | 27.115 | 52.585 | −26.066 | 1.00 | 12.98 | A |
| ATOM | 6590 | OE1 | GLU | A | 838 | 26.983 | 51.612 | −25.296 | 1.00 | 15.20 | A |
| ATOM | 6591 | OE2 | GLU | A | 838 | 28.121 | 52.737 | −26.784 | 1.00 | 15.16 | A |
| ATOM | 6592 | C | GLU | A | 838 | 23.805 | 54.978 | −27.484 | 1.00 | 10.72 | A |
| ATOM | 6593 | O | GLU | A | 838 | 22.957 | 54.747 | −26.626 | 1.00 | 10.48 | A |
| ATOM | 6594 | N | ASP | A | 839 | 23.675 | 54.621 | −28.754 | 1.00 | 11.70 | A |
| ATOM | 6595 | CA | ASP | A | 839 | 22.577 | 53.778 | −29.192 | 1.00 | 12.02 | A |
| ATOM | 6596 | CB | ASP | A | 839 | 21.508 | 54.533 | −30.004 | 1.00 | 12.02 | A |
| ATOM | 6597 | CG | ASP | A | 839 | 22.056 | 55.233 | −31.231 | 1.00 | 11.88 | A |
| ATOM | 6598 | OD1 | ASP | A | 839 | 23.063 | 54.777 | −31.810 | 1.00 | 13.51 | A |
| ATOM | 6599 | OD2 | ASP | A | 839 | 21.440 | 56.247 | −31.629 | 1.00 | 15.05 | A |
| ATOM | 6600 | C | ASP | A | 839 | 23.267 | 52.704 | −30.014 | 1.00 | 12.39 | A |
| ATOM | 6601 | O | ASP | A | 839 | 24.476 | 52.517 | −29.891 | 1.00 | 11.96 | A |
| ATOM | 6602 | N | ALA | A | 840 | 22.525 | 51.990 | −30.846 | 1.00 | 12.91 | A |
| ATOM | 6603 | CA | ALA | A | 840 | 23.136 | 50.931 | −31.634 | 1.00 | 13.57 | A |
| ATOM | 6604 | CB | ALA | A | 840 | 22.058 | 50.198 | −32.427 | 1.00 | 13.53 | A |
| ATOM | 6605 | C | ALA | A | 840 | 24.245 | 51.387 | −32.579 | 1.00 | 13.48 | A |
| ATOM | 6606 | O | ALA | A | 840 | 25.205 | 50.650 | −32.815 | 1.00 | 14.95 | A |
| ATOM | 6607 | N | ASN | A | 841 | 24.138 | 52.606 | −33.100 | 1.00 | 13.29 | A |
| ATOM | 6608 | CA | ASN | A | 841 | 25.118 | 53.083 | −34.073 | 1.00 | 12.90 | A |
| ATOM | 6609 | CB | ASN | A | 841 | 24.404 | 53.445 | −35.377 | 1.00 | 14.72 | A |
| ATOM | 6610 | CG | ASN | A | 841 | 23.583 | 52.304 | −35.927 | 1.00 | 15.43 | A |
| ATOM | 6611 | OD1 | ASN | A | 841 | 24.092 | 51.207 | −36.136 | 1.00 | 18.11 | A |
| ATOM | 6612 | ND2 | ASN | A | 841 | 22.301 | 52.560 | −36.167 | 1.00 | 17.87 | A |
| ATOM | 6613 | C | ASN | A | 841 | 26.011 | 54.258 | −33.718 | 1.00 | 12.20 | A |
| ATOM | 6614 | O | ASN | A | 841 | 27.100 | 54.389 | −34.274 | 1.00 | 12.69 | A |
| ATOM | 6615 | N | THR | A | 842 | 25.557 | 55.111 | −32.810 | 1.00 | 11.81 | A |
| ATOM | 6616 | CA | THR | A | 842 | 26.317 | 56.307 | −32.485 | 1.00 | 11.57 | A |
| ATOM | 6617 | CB | THR | A | 842 | 25.569 | 57.557 | −33.001 | 1.00 | 11.76 | A |
| ATOM | 6618 | OG1 | THR | A | 842 | 25.148 | 57.338 | −34.354 | 1.00 | 13.92 | A |
| ATOM | 6619 | CG2 | THR | A | 842 | 26.470 | 58.786 | −32.954 | 1.00 | 11.70 | A |
| ATOM | 6620 | C | THR | A | 842 | 26.594 | 56.505 | −31.005 | 1.00 | 10.99 | A |
| ATOM | 6621 | O | THR | A | 842 | 25.796 | 56.123 | −30.154 | 1.00 | 11.90 | A |
| ATOM | 6622 | N | ARG | A | 843 | 27.738 | 57.116 | −30.712 | 1.00 | 10.93 | A |
| ATOM | 6623 | CA | ARG | A | 843 | 28.110 | 57.411 | −29.338 | 1.00 | 9.78 | A |
| ATOM | 6624 | CB | ARG | A | 843 | 29.132 | 56.403 | −28.801 | 1.00 | 10.05 | A |
| ATOM | 6625 | CG | ARG | A | 843 | 29.601 | 56.731 | −27.380 | 1.00 | 9.72 | A |
| ATOM | 6626 | CD | ARG | A | 843 | 30.786 | 55.880 | −26.941 | 1.00 | 9.07 | A |
| ATOM | 6627 | NE | ARG | A | 843 | 30.421 | 54.502 | −26.612 | 1.00 | 9.80 | A |
| ATOM | 6628 | CZ | ARG | A | 843 | 31.241 | 53.644 | −26.011 | 1.00 | 9.80 | A |
| ATOM | 6629 | NH1 | ARG | A | 843 | 32.469 | 54.019 | −25.675 | 1.00 | 9.81 | A |
| ATOM | 6630 | NH2 | ARG | A | 843 | 30.833 | 52.415 | −25.731 | 1.00 | 10.05 | A |
| ATOM | 6631 | C | ARG | A | 843 | 28.713 | 58.805 | −29.262 | 1.00 | 9.19 | A |
| ATOM | 6632 | O | ARG | A | 843 | 29.414 | 59.245 | −30.179 | 1.00 | 8.90 | A |
| ATOM | 6633 | N | LEU | A | 844 | 28.409 | 59.501 | −28.175 | 1.00 | 8.29 | A |
| ATOM | 6634 | CA | LEU | A | 844 | 28.956 | 60.826 | −27.930 | 1.00 | 7.96 | A |
| ATOM | 6635 | CB | LEU | A | 844 | 27.871 | 61.908 | −27.944 | 1.00 | 8.76 | A |
| ATOM | 6636 | CG | LEU | A | 844 | 28.466 | 63.319 | −27.799 | 1.00 | 9.51 | A |
| ATOM | 6637 | CD1 | LEU | A | 844 | 29.388 | 63.614 | −28.981 | 1.00 | 10.57 | A |
| ATOM | 6638 | CD2 | LEU | A | 844 | 27.353 | 64.350 | −27.716 | 1.00 | 11.46 | A |
| ATOM | 6639 | C | LEU | A | 844 | 29.597 | 60.755 | −26.555 | 1.00 | 7.93 | A |
| ATOM | 6640 | O | LEU | A | 844 | 28.924 | 60.463 | −25.561 | 1.00 | 7.88 | A |
| ATOM | 6641 | N | THR | A | 845 | 30.900 | 61.007 | −26.516 | 1.00 | 8.37 | A |
| ATOM | 6642 | CA | THR | A | 845 | 31.647 | 60.968 | −25.268 | 1.00 | 8.30 | A |
| ATOM | 6643 | CB | THR | A | 845 | 32.823 | 59.975 | −25.346 | 1.00 | 7.69 | A |
| ATOM | 6644 | OG1 | THR | A | 845 | 32.344 | 58.688 | −25.762 | 1.00 | 7.36 | A |
| ATOM | 6645 | CG2 | THR | A | 845 | 33.494 | 59.837 | −23.988 | 1.00 | 8.08 | A |
| ATOM | 6646 | C | THR | A | 845 | 32.220 | 62.342 | −24.949 | 1.00 | 8.06 | A |
| ATOM | 6647 | O | THR | A | 845 | 32.815 | 62.996 | −25.807 | 1.00 | 8.73 | A |
| ATOM | 6648 | N | LEU | A | 846 | 32.025 | 62.777 | −23.713 | 1.00 | 8.51 | A |
| ATOM | 6649 | CA | LEU | A | 846 | 32.552 | 64.054 | −23.264 | 1.00 | 7.79 | A |
| ATOM | 6650 | CB | LEU | A | 846 | 31.444 | 64.924 | −22.667 | 1.00 | 8.89 | A |
| ATOM | 6651 | CG | LEU | A | 846 | 31.893 | 66.296 | −22.158 | 1.00 | 9.02 | A |
| ATOM | 6652 | CD1 | LEU | A | 846 | 32.337 | 67.159 | −23.333 | 1.00 | 9.99 | A |
| ATOM | 6653 | CD2 | LEU | A | 846 | 30.751 | 66.964 | −21.402 | 1.00 | 11.17 | A |
| ATOM | 6654 | C | LEU | A | 846 | 33.583 | 63.733 | −22.193 | 1.00 | 7.73 | A |
| ATOM | 6655 | O | LEU | A | 846 | 33.241 | 63.165 | −21.155 | 1.00 | 7.34 | A |
| ATOM | 6656 | N | LEU | A | 847 | 34.843 | 64.057 | −22.467 | 1.00 | 7.27 | A |
| ATOM | 6657 | CA | LEU | A | 847 | 35.919 | 63.824 | −21.509 | 1.00 | 6.61 | A |
| ATOM | 6658 | CB | LEU | A | 847 | 37.194 | 63.357 | −22.214 | 1.00 | 7.48 | A |
| ATOM | 6659 | CG | LEU | A | 847 | 37.177 | 62.018 | −22.965 | 1.00 | 6.64 | A |
| ATOM | 6660 | CD1 | LEU | A | 847 | 36.564 | 60.942 | −22.078 | 1.00 | 7.20 | A |
| ATOM | 6661 | CD2 | LEU | A | 847 | 36.393 | 62.144 | −24.267 | 1.00 | 7.29 | A |
| ATOM | 6662 | C | LEU | A | 847 | 36.181 | 65.138 | −20.786 | 1.00 | 7.24 | A |
| ATOM | 6663 | O | LEU | A | 847 | 36.068 | 66.214 | −21.389 | 1.00 | 7.43 | A |
| ATOM | 6664 | N | THR | A | 848 | 36.531 | 65.058 | −19.503 | 1.00 | 6.70 | A |
| ATOM | 6665 | CA | THR | A | 848 | 36.781 | 66.268 | −18.716 | 1.00 | 8.11 | A |
| ATOM | 6666 | CB | THR | A | 848 | 35.796 | 66.393 | −17.537 | 1.00 | 9.99 | A |
| ATOM | 6667 | OG1 | THR | A | 848 | 36.158 | 65.457 | −16.510 | 1.00 | 12.32 | A |
| ATOM | 6668 | CG2 | THR | A | 848 | 34.383 | 66.109 | −17.986 | 1.00 | 10.74 | A |
| ATOM | 6669 | C | THR | A | 848 | 38.183 | 66.365 | −18.127 | 1.00 | 8.03 | A |
| ATOM | 6670 | O | THR | A | 848 | 38.852 | 65.354 | −17.886 | 1.00 | 8.97 | A |
| ATOM | 6671 | N | GLY | A | 849 | 38.620 | 67.598 | −17.891 | 1.00 | 7.93 | A |
| ATOM | 6672 | CA | GLY | A | 849 | 39.923 | 67.822 | −17.298 | 1.00 | 7.56 | A |
| ATOM | 6673 | C | GLY | A | 849 | 39.761 | 68.195 | −15.836 | 1.00 | 7.38 | A |
| ATOM | 6674 | O | GLY | A | 849 | 40.708 | 68.628 | −15.182 | 1.00 | 7.22 | A |
| ATOM | 6675 | N | GLN | A | 850 | 38.544 | 68.027 | −15.328 | 1.00 | 6.67 | A |
| ATOM | 6676 | CA | GLN | A | 850 | 38.219 | 68.334 | −13.940 | 1.00 | 7.10 | A |
| ATOM | 6677 | CB | GLN | A | 850 | 38.021 | 69.847 | −13.759 | 1.00 | 7.43 | A |
| ATOM | 6678 | CG | GLN | A | 850 | 36.891 | 70.455 | −14.612 | 1.00 | 7.11 | A |
| ATOM | 6679 | CD | GLN | A | 850 | 37.239 | 70.552 | −16.086 | 1.00 | 7.63 | A |
| ATOM | 6680 | OE1 | GLN | A | 850 | 38.338 | 70.967 | −16.454 | 1.00 | 8.22 | A |
| ATOM | 6681 | NE2 | GLN | A | 850 | 36.289 | 70.183 | −16.945 | 1.00 | 7.13 | A |
| ATOM | 6682 | C | GLN | A | 850 | 36.939 | 67.601 | −13.543 | 1.00 | 7.56 | A |
| ATOM | 6683 | O | GLN | A | 850 | 36.058 | 67.387 | −14.375 | 1.00 | 7.52 | A |
| ATOM | 6684 | N | PRO | A | 851 | 36.827 | 67.194 | −12.269 | 1.00 | 6.72 | A |
| ATOM | 6685 | CD | PRO | A | 851 | 37.849 | 67.209 | −11.207 | 1.00 | 6.45 | A |
| ATOM | 6686 | CA | PRO | A | 851 | 35.619 | 66.492 | −11.829 | 1.00 | 6.46 | A |
| ATOM | 6687 | CB | PRO | A | 851 | 36.060 | 65.825 | −10.525 | 1.00 | 6.15 | A |
| ATOM | 6688 | CG | PRO | A | 851 | 37.056 | 66.810 | −9.976 | 1.00 | 6.55 | A |
| ATOM | 6689 | C | PRO | A | 851 | 34.473 | 67.474 | −11.623 | 1.00 | 7.19 | A |
| ATOM | 6690 | O | PRO | A | 851 | 34.643 | 68.516 | −10.982 | 1.00 | 7.03 | A |
| ATOM | 6691 | N | LEU | A | 852 | 33.316 | 67.144 | −12.190 | 1.00 | 7.54 | A |
| ATOM | 6692 | CA | LEU | A | 852 | 32.120 | 67.977 | −12.083 | 1.00 | 8.39 | A |
| ATOM | 6693 | CB | LEU | A | 852 | 32.022 | 68.920 | −13.290 | 1.00 | 8.88 | A |
| ATOM | 6694 | CG | LEU | A | 852 | 33.166 | 69.925 | −13.485 | 1.00 | 8.62 | A |
| ATOM | 6695 | CD1 | LEU | A | 852 | 33.096 | 70.522 | −14.891 | 1.00 | 9.44 | A |
| ATOM | 6696 | CD2 | LEU | A | 852 | 33.089 | 71.015 | −12.431 | 1.00 | 8.95 | A |
| ATOM | 6697 | C | LEU | A | 852 | 30.905 | 67.055 | −12.048 | 1.00 | 8.99 | A |
| ATOM | 6698 | O | LEU | A | 852 | 31.022 | 65.872 | −12.351 | 1.00 | 10.89 | A |
| ATOM | 6699 | N | GLY | A | 853 | 29.750 | 67.593 | −11.673 | 1.00 | 7.59 | A |
| ATOM | 6700 | CA | GLY | A | 853 | 28.545 | 66.781 | −11.619 | 1.00 | 7.39 | A |
| ATOM | 6701 | C | GLY | A | 853 | 27.827 | 66.774 | −12.955 | 1.00 | 7.76 | A |
| ATOM | 6702 | O | GLY | A | 853 | 27.871 | 67.760 | −13.699 | 1.00 | 7.36 | A |
| ATOM | 6703 | N | GLY | A | 854 | 27.157 | 65.672 | −13.273 | 1.00 | 6.90 | A |
| ATOM | 6704 | CA | GLY | A | 854 | 26.458 | 65.610 | −14.543 | 1.00 | 8.09 | A |
| ATOM | 6705 | C | GLY | A | 854 | 25.406 | 64.527 | −14.623 | 1.00 | 7.89 | A |
| ATOM | 6706 | O | GLY | A | 854 | 25.239 | 63.734 | −13.695 | 1.00 | 8.50 | A |
| ATOM | 6707 | N | SER | A | 855 | 24.703 | 64.480 | −15.749 | 1.00 | 8.37 | A |
| ATOM | 6708 | CA | SER | A | 855 | 23.649 | 63.494 | −15.931 | 1.00 | 9.33 | A |
| ATOM | 6709 | CB | SER | A | 855 | 22.461 | 63.853 | −15.032 | 1.00 | 10.99 | A |
| ATOM | 6710 | OG | SER | A | 855 | 21.407 | 62.906 | −15.128 | 1.00 | 10.64 | A |
| ATOM | 6711 | C | SER | A | 855 | 23.186 | 63.483 | −17.378 | 1.00 | 9.38 | A |
| ATOM | 6712 | O | SER | A | 855 | 23.755 | 64.156 | −18.237 | 1.00 | 9.01 | A |
| ATOM | 6713 | N | SER | A | 856 | 22.153 | 62.687 | −17.629 | 1.00 | 9.89 | A |
| ATOM | 6714 | CA | SER | A | 856 | 21.502 | 62.599 | −18.933 | 1.00 | 10.04 | A |
| ATOM | 6715 | CB | SER | A | 856 | 21.872 | 61.313 | −19.670 | 1.00 | 9.85 | A |
| ATOM | 6716 | OG | SER | A | 856 | 21.144 | 61.223 | −20.889 | 1.00 | 10.25 | A |
| ATOM | 6717 | C | SER | A | 856 | 20.041 | 62.560 | −18.515 | 1.00 | 10.83 | A |
| ATOM | 6718 | O | SER | A | 856 | 19.507 | 61.496 | −18.203 | 1.00 | 11.04 | A |
| ATOM | 6719 | N | LEU | A | 857 | 19.405 | 63.729 | −18.491 | 1.00 | 11.03 | A |
| ATOM | 6720 | CA | LEU | A | 857 | 18.020 | 63.834 | −18.051 | 1.00 | 11.33 | A |
| ATOM | 6721 | CB | LEU | A | 857 | 17.776 | 65.233 | −17.475 | 1.00 | 12.21 | A |
| ATOM | 6722 | CG | LEU | A | 857 | 18.613 | 65.514 | −16.224 | 1.00 | 12.41 | A |
| ATOM | 6723 | CD1 | LEU | A | 857 | 18.420 | 66.959 | −15.772 | 1.00 | 12.54 | A |
| ATOM | 6724 | CD2 | LEU | A | 857 | 18.213 | 64.538 | −15.117 | 1.00 | 13.38 | A |
| ATOM | 6725 | C | LEU | A | 857 | 16.970 | 63.499 | −19.099 | 1.00 | 11.41 | A |
| ATOM | 6726 | O | LEU | A | 857 | 15.775 | 63.478 | −18.800 | 1.00 | 12.16 | A |
| ATOM | 6727 | N | ALA | A | 858 | 17.422 | 63.235 | −20.320 | 1.00 | 10.23 | A |
| ATOM | 6728 | CA | ALA | A | 858 | 16.532 | 62.871 | −21.417 | 1.00 | 10.98 | A |
| ATOM | 6729 | CB | ALA | A | 858 | 15.852 | 64.117 | −21.999 | 1.00 | 10.30 | A |
| ATOM | 6730 | C | ALA | A | 858 | 17.333 | 62.156 | −22.494 | 1.00 | 11.53 | A |
| ATOM | 6731 | O | ALA | A | 858 | 18.530 | 62.395 | −22.655 | 1.00 | 11.99 | A |
| ATOM | 6732 | N | SER | A | 859 | 16.667 | 61.270 | −23.226 | 1.00 | 11.71 | A |
| ATOM | 6733 | CA | SER | A | 859 | 17.314 | 60.518 | −24.287 | 1.00 | 11.84 | A |
| ATOM | 6734 | CB | SER | A | 859 | 16.265 | 59.722 | −25.077 | 1.00 | 12.18 | A |
| ATOM | 6735 | OG | SER | A | 859 | 16.863 | 58.942 | −26.094 | 1.00 | 13.57 | A |
| ATOM | 6736 | C | SER | A | 859 | 18.063 | 61.465 | −25.218 | 1.00 | 11.69 | A |
| ATOM | 6737 | O | SER | A | 859 | 17.535 | 62.508 | −25.612 | 1.00 | 12.09 | A |
| ATOM | 6738 | N | GLY | A | 860 | 19.299 | 61.101 | −25.542 | 1.00 | 11.53 | A |
| ATOM | 6739 | CA | GLY | A | 860 | 20.117 | 61.904 | −26.435 | 1.00 | 11.19 | A |
| ATOM | 6740 | C | GLY | A | 860 | 20.846 | 63.073 | −25.798 | 1.00 | 11.05 | A |
| ATOM | 6741 | O | GLY | A | 860 | 21.585 | 63.780 | −26.483 | 1.00 | 10.94 | A |
| ATOM | 6742 | N | GLU | A | 861 | 20.665 | 63.276 | −24.496 | 1.00 | 10.54 | A |
| ATOM | 6743 | CA | GLU | A | 861 | 21.318 | 64.390 | −23.812 | 1.00 | 10.72 | A |
| ATOM | 6744 | CB | GLU | A | 861 | 20.309 | 65.181 | −22.981 | 1.00 | 11.69 | A |
| ATOM | 6745 | CG | GLU | A | 861 | 19.143 | 65.764 | −23.742 | 1.00 | 13.84 | A |
| ATOM | 6746 | CD | GLU | A | 861 | 18.277 | 66.649 | −22.863 | 1.00 | 14.55 | A |
| ATOM | 6747 | OE1 | GLU | A | 861 | 18.521 | 66.706 | −21.638 | 1.00 | 15.28 | A |
| ATOM | 6748 | OE2 | GLU | A | 861 | 17.348 | 67.291 | −23.395 | 1.00 | 16.06 | A |
| ATOM | 6749 | C | GLU | A | 861 | 22.460 | 64.020 | −22.874 | 1.00 | 10.67 | A |
| ATOM | 6750 | O | GLU | A | 861 | 22.543 | 62.907 | −22.357 | 1.00 | 10.90 | A |
| ATOM | 6751 | N | LED | A | 862 | 23.333 | 64.998 | −22.665 | 1.00 | 10.06 | A |
| ATOM | 6752 | CA | LEU | A | 862 | 24.455 | 64.894 | −21.736 | 1.00 | 9.88 | A |
| ATOM | 6753 | CB | LEU | A | 862 | 25.775 | 64.590 | −22.451 | 1.00 | 9.80 | A |
| ATOM | 6754 | CG | LEU | A | 862 | 26.088 | 63.195 | −22.990 | 1.00 | 9.38 | A |
| ATOM | 6755 | CD1 | LEU | A | 862 | 27.467 | 63.212 | −23.644 | 1.00 | 8.93 | A |
| ATOM | 6756 | CD2 | LEU | A | 862 | 26.054 | 62.182 | −21.852 | 1.00 | 10.32 | A |
| ATOM | 6757 | C | LEU | A | 862 | 24.553 | 66.286 | −21.143 | 1.00 | 9.97 | A |
| ATOM | 6758 | O | LEU | A | 862 | 24.420 | 67.272 | −21.865 | 1.00 | 10.40 | A |
| ATOM | 6759 | N | GLU | A | 863 | 24.748 | 66.387 | −19.838 | 1.00 | 9.17 | A |
| ATOM | 6760 | CA | GLU | A | 863 | 24.905 | 67.705 | −19.243 | 1.00 | 8.61 | A |
| ATOM | 6761 | CB | GLU | A | 863 | 23.562 | 68.289 | −18.791 | 1.00 | 9.86 | A |
| ATOM | 6762 | CG | GLU | A | 863 | 22.976 | 67.732 | −17.514 | 1.00 | 9.77 | A |
| ATOM | 6763 | CD | GLU | A | 863 | 21.678 | 68.431 | −17.154 | 1.00 | 10.41 | A |
| ATOM | 6764 | OE1 | GLU | A | 863 | 21.570 | 68.956 | −16.028 | 1.00 | 10.63 | A |
| ATOM | 6765 | OE2 | GLU | A | 863 | 20.764 | 68.458 | −18.008 | 1.00 | 11.95 | A |
| ATOM | 6766 | C | GLU | A | 863 | 25.878 | 67.629 | −18.090 | 1.00 | 9.12 | A |
| ATOM | 6767 | O | GLU | A | 863 | 25.975 | 66.608 | −17.408 | 1.00 | 7.97 | A |
| ATOM | 6768 | N | ILE | A | 864 | 26.612 | 68.713 | −17.887 | 1.00 | 9.26 | A |
| ATOM | 6769 | CA | ILE | A | 864 | 27.608 | 68.757 | −16.835 | 1.00 | 9.59 | A |
| ATOM | 6770 | CB | ILE | A | 864 | 28.987 | 68.324 | −17.416 | 1.00 | 9.91 | A |
| ATOM | 6771 | CG2 | ILE | A | 864 | 29.401 | 69.265 | −18.541 | 1.00 | 10.24 | A |
| ATOM | 6772 | CG1 | ILE | A | 864 | 30.040 | 68.250 | −16.308 | 1.00 | 10.77 | A |
| ATOM | 6773 | CD1 | ILE | A | 864 | 31.299 | 67.497 | −16.735 | 1.00 | 11.71 | A |
| ATOM | 6774 | C | ILE | A | 864 | 27.649 | 70.161 | −16.234 | 1.00 | 9.24 | A |
| ATOM | 6775 | O | ILE | A | 864 | 27.713 | 71.164 | −16.956 | 1.00 | 9.45 | A |
| ATOM | 6776 | N | MSE | A | 865 | 27.580 | 70.226 | −14.908 | 1.00 | 8.75 | A |
| ATOM | 6777 | CA | MSE | A | 865 | 27.580 | 71.496 | −14.193 | 1.00 | 9.06 | A |
| ATOM | 6778 | CB | MSE | A | 865 | 27.176 | 71.269 | −12.733 | 1.00 | 11.05 | A |
| ATOM | 6779 | CG | MSE | A | 865 | 26.538 | 72.478 | −12.066 | 1.00 | 11.46 | A |
| ATOM | 6780 | SE | MSE | A | 865 | 24.827 | 72.915 | −12.863 | 1.00 | 18.59 | A |
| ATOM | 6781 | CE | MSE | A | 865 | 23.751 | 71.627 | −11.924 | 1.00 | 12.98 | A |
| ATOM | 6782 | C | MSE | A | 865 | 28.936 | 72.200 | −14.257 | 1.00 | 9.30 | A |
| ATOM | 6783 | O | MSE | A | 865 | 29.984 | 71.569 | −14.105 | 1.00 | 9.37 | A |
| ATOM | 6784 | N | GLN | A | 866 | 28.903 | 73.514 | −14.467 | 1.00 | 9.20 | A |
| ATOM | 6785 | CA | GLN | A | 866 | 30.121 | 74.316 | −14.581 | 1.00 | 8.74 | A |
| ATOM | 6786 | CB | GLN | A | 866 | 29.942 | 75.368 | −15.673 | 1.00 | 9.56 | A |
| ATOM | 6787 | CG | GLN | A | 866 | 29.577 | 74.765 | −17.013 | 1.00 | 9.63 | A |
| ATOM | 6788 | CD | GLN | A | 866 | 30.598 | 73.749 | −17.470 | 1.00 | 9.75 | A |
| ATOM | 6789 | OE1 | GLN | A | 866 | 31.725 | 74.097 | −17.823 | 1.00 | 10.41 | A |
| ATOM | 6790 | NE2 | GLN | A | 866 | 30.215 | 72.479 | −17.449 | 1.00 | 9.11 | A |
| ATOM | 6791 | C | GLN | A | 866 | 30.513 | 74.986 | −13.271 | 1.00 | 9.34 | A |
| ATOM | 6792 | O | GLN | A | 866 | 31.686 | 75.021 | −12.910 | 1.00 | 9.31 | A |
| ATOM | 6793 | N | ASP | A | 867 | 29.529 | 75.549 | −12.580 | 1.00 | 9.02 | A |
| ATOM | 6794 | CA | ASP | A | 867 | 29.770 | 76.177 | −11.288 | 1.00 | 9.28 | A |
| ATOM | 6795 | CB | ASP | A | 867 | 30.539 | 77.495 | −11.430 | 1.00 | 9.80 | A |
| ATOM | 6796 | CG | ASP | A | 867 | 31.224 | 77.920 | −10.128 | 1.00 | 9.59 | A |
| ATOM | 6797 | OD1 | ASP | A | 867 | 31.051 | 77.242 | −9.089 | 1.00 | 9.81 | A |
| ATOM | 6798 | OD2 | ASP | A | 867 | 31.944 | 78.937 | −10.144 | 1.00 | 11.47 | A |
| ATOM | 6799 | C | ASP | A | 867 | 28.430 | 76.428 | −10.630 | 1.00 | 9.67 | A |
| ATOM | 6800 | O | ASP | A | 867 | 27.381 | 76.361 | −11.281 | 1.00 | 10.05 | A |
| ATOM | 6801 | N | ARG | A | 868 | 28.470 | 76.705 | −9.335 | 1.00 | 10.02 | A |
| ATOM | 6802 | CA | ARG | A | 868 | 27.263 | 76.958 | −8.573 | 1.00 | 10.34 | A |
| ATOM | 6803 | CB | ARG | A | 868 | 26.748 | 75.646 | −7.969 | 1.00 | 10.15 | A |
| ATOM | 6804 | CG | ARG | A | 868 | 27.773 | 74.880 | −7.138 | 1.00 | 10.84 | A |
| ATOM | 6805 | CD | ARG | A | 868 | 27.464 | 73.380 | −7.140 | 1.00 | 10.25 | A |
| ATOM | 6806 | NE | ARG | A | 868 | 26.101 | 73.108 | −6.696 | 1.00 | 9.69 | A |
| ATOM | 6807 | CZ | ARG | A | 868 | 25.761 | 72.818 | −5.444 | 1.00 | 9.26 | A |
| ATOM | 6808 | NH1 | ARG | A | 868 | 26.687 | 72.743 | −4.496 | 1.00 | 8.58 | A |
| ATOM | 6809 | NH2 | ARG | A | 868 | 24.486 | 72.628 | −5.136 | 1.00 | 9.51 | A |
| ATOM | 6810 | C | ARG | A | 868 | 27.570 | 77.977 | −7.486 | 1.00 | 10.82 | A |
| ATOM | 6811 | O | ARG | A | 868 | 28.606 | 77.901 | −6.825 | 1.00 | 11.25 | A |
| ATOM | 6812 | N | ARG | A | 869 | 26.671 | 78.944 | −7.333 | 1.00 | 10.70 | A |
| ATOM | 6813 | CA | ARG | A | 869 | 26.816 | 80.003 | −6.337 | 1.00 | 11.61 | A |
| ATOM | 6814 | CB | ARG | A | 869 | 26.990 | 81.357 | −7.037 | 1.00 | 11.61 | A |
| ATOM | 6815 | CG | ARG | A | 869 | 27.262 | 82.532 | −6.103 | 1.00 | 12.74 | A |
| ATOM | 6816 | CD | ARG | A | 869 | 27.557 | 83.804 | −6.889 | 1.00 | 13.91 | A |
| ATOM | 6817 | NE | ARG | A | 869 | 27.739 | 84.974 | −6.029 | 1.00 | 15.08 | A |
| ATOM | 6818 | CZ | ARG | A | 869 | 28.853 | 85.263 | −5.364 | 1.00 | 15.70 | A |
| ATOM | 6819 | NH1 | ARG | A | 869 | 29.911 | 84.470 | −5.451 | 1.00 | 16.09 | A |
| ATOM | 6820 | NH2 | ARG | A | 869 | 28.907 | 86.351 | −4.605 | 1.00 | 17.21 | A |
| ATOM | 6821 | C | ARG | A | 869 | 25.525 | 79.962 | −5.531 | 1.00 | 12.83 | A |
| ATOM | 6822 | O | ARG | A | 869 | 24.445 | 80.221 | −6.061 | 1.00 | 12.65 | A |
| ATOM | 6823 | N | LEU | A | 870 | 25.650 | 79.610 | −4.255 | 1.00 | 13.19 | A |
| ATOM | 6824 | CA | LEU | A | 870 | 24.505 | 79.472 | −3.357 | 1.00 | 14.88 | A |
| ATOM | 6825 | CB | LEU | A | 870 | 24.395 | 78.012 | −2.934 | 1.00 | 14.84 | A |
| ATOM | 6826 | CG | LEU | A | 870 | 24.440 | 77.060 | −4.132 | 1.00 | 16.12 | A |
| ATOM | 6827 | CD1 | LEU | A | 870 | 24.778 | 75.671 | −3.659 | 1.00 | 16.16 | A |
| ATOM | 6828 | CD2 | LEU | A | 870 | 23.116 | 77.092 | −4.880 | 1.00 | 15.69 | A |
| ATOM | 6829 | C | LEU | A | 870 | 24.603 | 80.362 | −2.123 | 1.00 | 15.84 | A |
| ATOM | 6830 | O | LEU | A | 870 | 25.596 | 80.333 | −1.396 | 1.00 | 15.42 | A |
| ATOM | 6831 | N | ALA | A | 871 | 23.548 | 81.131 | −1.877 | 1.00 | 17.32 | A |
| ATOM | 6832 | CA | ALA | A | 871 | 23.521 | 82.055 | −0.752 | 1.00 | 19.19 | A |
| ATOM | 6833 | CB | ALA | A | 871 | 22.497 | 83.152 | −1.023 | 1.00 | 19.49 | A |
| ATOM | 6834 | C | ALA | A | 871 | 23.250 | 81.427 | 0.611 | 1.00 | 20.12 | A |
| ATOM | 6835 | O | ALA | A | 871 | 23.687 | 81.954 | 1.634 | 1.00 | 21.06 | A |
| ATOM | 6836 | N | SER | A | 872 | 22.546 | 80.303 | 0.640 | 1.00 | 20.24 | A |
| ATOM | 6837 | CA | SER | A | 872 | 22.227 | 79.684 | 1.921 | 1.00 | 20.96 | A |
| ATOM | 6838 | CB | SER | A | 872 | 20.729 | 79.374 | 1.988 | 1.00 | 22.69 | A |
| ATOM | 6839 | OG | SER | A | 872 | 20.354 | 78.455 | 0.981 | 1.00 | 26.69 | A |
| ATOM | 6840 | C | SER | A | 872 | 23.016 | 78.430 | 2.275 | 1.00 | 20.07 | A |
| ATOM | 6841 | O | SER | A | 872 | 23.619 | 77.782 | 1.418 | 1.00 | 19.32 | A |
| ATOM | 6842 | N | ASP | A | 873 | 23.009 | 78.115 | 3.567 | 1.00 | 19.80 | A |
| ATOM | 6843 | CA | ASP | A | 873 | 23.680 | 76.938 | 4.105 | 1.00 | 19.26 | A |
| ATOM | 6844 | CB | ASP | A | 873 | 24.046 | 77.187 | 5.572 | 1.00 | 19.50 | A |
| ATOM | 6845 | CG | ASP | A | 873 | 24.426 | 75.918 | 6.308 | 1.00 | 19.97 | A |
| ATOM | 6846 | OD1 | ASP | A | 873 | 23.546 | 75.332 | 6.977 | 1.00 | 19.80 | A |
| ATOM | 6847 | OD2 | ASP | A | 873 | 25.602 | 75.505 | 6.214 | 1.00 | 20.62 | A |
| ATOM | 6848 | C | ASP | A | 873 | 22.697 | 75.779 | 3.979 | 1.00 | 18.67 | A |
| ATOM | 6849 | O | ASP | A | 873 | 21.488 | 75.985 | 4.077 | 1.00 | 18.83 | A |
| ATOM | 6850 | N | ASP | A | 874 | 23.201 | 74.568 | 3.752 | 1.00 | 17.49 | A |
| ATOM | 6851 | CA | ASP | A | 874 | 22.318 | 73.417 | 3.602 | 1.00 | 16.56 | A |
| ATOM | 6852 | CB | ASP | A | 874 | 22.678 | 72.623 | 2.338 | 1.00 | 15.33 | A |
| ATOM | 6853 | CG | ASP | A | 874 | 24.160 | 72.341 | 2.217 | 1.00 | 15.79 | A |
| ATOM | 6854 | OD1 | ASP | A | 874 | 24.932 | 72.741 | 3.115 | 1.00 | 14.72 | A |
| ATOM | 6855 | OD2 | ASP | A | 874 | 24.547 | 71.716 | 1.210 | 1.00 | 13.54 | A |
| ATOM | 6856 | C | ASP | A | 874 | 22.226 | 72.486 | 4.809 | 1.00 | 17.23 | A |
| ATOM | 6857 | O | ASP | A | 874 | 22.119 | 71.269 | 4.668 | 1.00 | 17.24 | A |
| ATOM | 6858 | N | GLU | A | 875 | 22.270 | 73.083 | 5.995 | 1.00 | 17.61 | A |
| ATOM | 6859 | CA | GLU | A | 875 | 22.132 | 72.370 | 7.261 | 1.00 | 18.30 | A |
| ATOM | 6860 | CB | GLU | A | 875 | 20.653 | 72.025 | 7.481 | 1.00 | 21.02 | A |
| ATOM | 6861 | CG | GLU | A | 875 | 19.741 | 73.249 | 7.502 | 1.00 | 24.64 | A |
| ATOM | 6862 | CD | GLU | A | 875 | 18.302 | 72.916 | 7.858 | 1.00 | 26.93 | A |
| ATOM | 6863 | OE1 | GLU | A | 875 | 18.069 | 72.346 | 8.945 | 1.00 | 28.85 | A |
| ATOM | 6864 | OE2 | GLU | A | 875 | 17.401 | 73.230 | 7.053 | 1.00 | 29.01 | A |
| ATOM | 6865 | C | GLU | A | 875 | 22.977 | 71.123 | 7.503 | 1.00 | 17.56 | A |
| ATOM | 6866 | O | GLU | A | 875 | 22.499 | 70.160 | 8.109 | 1.00 | 17.20 | A |
| ATOM | 6867 | N | ARG | A | 876 | 24.226 | 71.130 | 7.051 | 1.00 | 16.12 | A |
| ATOM | 6868 | CA | ARG | A | 876 | 25.091 | 69.980 | 7.285 | 1.00 | 15.68 | A |
| ATOM | 6869 | CB | ARG | A | 876 | 25.519 | 69.343 | 5.955 | 1.00 | 14.98 | A |
| ATOM | 6870 | CG | ARG | A | 876 | 24.365 | 68.665 | 5.196 | 1.00 | 14.42 | A |
| ATOM | 6871 | CD | ARG | A | 876 | 23.701 | 67.581 | 6.046 | 1.00 | 13.74 | A |
| ATOM | 6872 | NE | ARG | A | 876 | 22.636 | 66.850 | 5.354 | 1.00 | 13.11 | A |
| ATOM | 6873 | CZ | ARG | A | 876 | 21.460 | 67.366 | 5.004 | 1.00 | 13.22 | A |
| ATOM | 6874 | NH1 | ARG | A | 876 | 21.179 | 68.636 | 5.267 | 1.00 | 12.68 | A |
| ATOM | 6875 | NH2 | ARG | A | 876 | 20.548 | 66.601 | 4.411 | 1.00 | 12.89 | A |
| ATOM | 6876 | C | ARG | A | 876 | 26.307 | 70.364 | 8.130 | 1.00 | 15.55 | A |
| ATOM | 6877 | O | ARG | A | 876 | 27.273 | 69.605 | 8.231 | 1.00 | 16.13 | A |
| ATOM | 6878 | N | GLY | A | 877 | 26.249 | 71.548 | 8.738 | 1.00 | 15.42 | A |
| ATOM | 6879 | CA | GLY | A | 877 | 27.328 | 71.996 | 9.601 | 1.00 | 14.96 | A |
| ATOM | 6880 | C | GLY | A | 877 | 28.340 | 72.984 | 9.049 | 1.00 | 14.99 | A |
| ATOM | 6881 | O | GLY | A | 877 | 29.112 | 73.563 | 9.818 | 1.00 | 14.85 | A |
| ATOM | 6882 | N | LEU | A | 878 | 28.344 | 73.192 | 7.736 | 1.00 | 14.62 | A |
| ATOM | 6883 | CA | LEU | A | 878 | 29.299 | 74.113 | 7.122 | 1.00 | 15.39 | A |
| ATOM | 6884 | CB | LEU | A | 878 | 29.168 | 74.072 | 5.597 | 1.00 | 14.87 | A |
| ATOM | 6885 | CG | LEU | A | 878 | 30.078 | 74.997 | 4.782 | 1.00 | 15.40 | A |
| ATOM | 6886 | CD1 | LEU | A | 878 | 31.532 | 74.844 | 5.224 | 1.00 | 15.22 | A |
| ATOM | 6887 | CD2 | LEU | A | 878 | 29.928 | 74.663 | 3.308 | 1.00 | 15.32 | A |
| ATOM | 6888 | C | LEU | A | 878 | 29.134 | 75.543 | 7.630 | 1.00 | 15.66 | A |
| ATOM | 6889 | O | LEU | A | 878 | 30.119 | 76.257 | 7.823 | 1.00 | 15.76 | A |
| ATOM | 6890 | N | GLY | A | 879 | 27.888 | 75.957 | 7.843 | 1.00 | 15.47 | A |
| ATOM | 6891 | CA | GLY | A | 879 | 27.631 | 77.293 | 8.355 | 1.00 | 16.15 | A |
| ATOM | 6892 | C | GLY | A | 879 | 27.788 | 78.428 | 7.361 | 1.00 | 16.58 | A |
| ATOM | 6893 | O | GLY | A | 879 | 27.968 | 79.580 | 7.755 | 1.00 | 17.29 | A |
| ATOM | 6894 | N | GLN | A | 880 | 27.729 | 78.108 | 6.074 | 1.00 | 16.52 | A |
| ATOM | 6895 | CA | GLN | A | 880 | 27.843 | 79.125 | 5.035 | 1.00 | 16.57 | A |
| ATOM | 6896 | CB | GLN | A | 880 | 29.284 | 79.648 | 4.919 | 1.00 | 16.88 | A |
| ATOM | 6897 | CG | GLN | A | 880 | 30.329 | 78.599 | 4.526 | 1.00 | 17.13 | A |
| ATOM | 6898 | CD | GLN | A | 880 | 31.561 | 79.211 | 3.873 | 1.00 | 17.00 | A |
| ATOM | 6899 | OE1 | GLN | A | 880 | 31.577 | 79.470 | 2.665 | 1.00 | 18.68 | A |
| ATOM | 6900 | NE2 | GLN | A | 880 | 32.594 | 79.458 | 4.668 | 1.00 | 15.41 | A |
| ATOM | 6901 | C | GLN | A | 880 | 27.415 | 78.556 | 3.696 | 1.00 | 16.89 | A |
| ATOM | 6902 | O | GLN | A | 880 | 27.350 | 77.338 | 3.519 | 1.00 | 17.17 | A |
| ATOM | 6903 | N | GLY | A | 881 | 27.104 | 79.447 | 2.762 | 1.00 | 17.12 | A |
| ATOM | 6904 | CA | GLY | A | 881 | 26.725 | 79.010 | 1.436 | 1.00 | 16.41 | A |
| ATOM | 6905 | C | GLY | A | 881 | 28.008 | 78.947 | 0.634 | 1.00 | 15.96 | A |
| ATOM | 6906 | O | GLY | A | 881 | 29.092 | 78.817 | 1.203 | 1.00 | 16.71 | A |
| ATOM | 6907 | N | VAL | A | 882 | 27.893 | 79.037 | −0.682 | 1.00 | 14.79 | A |
| ATOM | 6908 | CA | VAL | A | 882 | 29.060 | 79.011 | −1.552 | 1.00 | 14.14 | A |
| ATOM | 6909 | CB | VAL | A | 882 | 28.998 | 77.814 | −2.520 | 1.00 | 13.86 | A |
| ATOM | 6910 | CG1 | VAL | A | 882 | 30.185 | 77.843 | −3.463 | 1.00 | 13.33 | A |
| ATOM | 6911 | CG2 | VAL | A | 882 | 28.987 | 76.517 | −1.726 | 1.00 | 14.87 | A |
| ATOM | 6912 | C | VAL | A | 882 | 29.029 | 80.320 | −2.328 | 1.00 | 13.86 | A |
| ATOM | 6913 | O | VAL | A | 882 | 28.353 | 80.432 | −3.348 | 1.00 | 13.36 | A |
| ATOM | 6914 | N | LEU | A | 883 | 29.755 | 81.312 | −1.823 | 1.00 | 13.97 | A |
| ATOM | 6915 | CA | LEU | A | 883 | 29.791 | 82.635 | −2.440 | 1.00 | 14.37 | A |
| ATOM | 6916 | CB | LEU | A | 883 | 29.119 | 83.650 | −1.505 | 1.00 | 14.55 | A |
| ATOM | 6917 | CG | LEU | A | 883 | 27.615 | 83.481 | −1.256 | 1.00 | 14.33 | A |
| ATOM | 6918 | CD1 | LEU | A | 883 | 27.175 | 84.299 | −0.052 | 1.00 | 14.92 | A |
| ATOM | 6919 | CD2 | LEU | A | 883 | 26.859 | 83.928 | −2.490 | 1.00 | 14.87 | A |
| ATOM | 6920 | C | LEU | A | 883 | 31.209 | 83.100 | −2.761 | 1.00 | 14.74 | A |
| ATOM | 6921 | O | LEU | A | 883 | 31.432 | 84.280 | −3.044 | 1.00 | 16.88 | A |
| ATOM | 6922 | N | ASP | A | 884 | 32.162 | 82.174 | −2.723 | 1.00 | 14.04 | A |
| ATOM | 6923 | CA | ASP | A | 884 | 33.555 | 82.500 | −2.998 | 1.00 | 14.10 | A |
| ATOM | 6924 | CB | ASP | A | 884 | 34.458 | 81.898 | −1.918 | 1.00 | 14.38 | A |
| ATOM | 6925 | CG | ASP | A | 884 | 34.250 | 80.402 | −1.737 | 1.00 | 15.01 | A |
| ATOM | 6926 | OD1 | ASP | A | 884 | 34.925 | 79.826 | −0.858 | 1.00 | 16.17 | A |
| ATOM | 6927 | OD2 | ASP | A | 884 | 33.421 | 79.803 | −2.461 | 1.00 | 13.93 | A |
| ATOM | 6928 | C | ASP | A | 884 | 34.021 | 82.044 | −4.376 | 1.00 | 13.67 | A |
| ATOM | 6929 | O | ASP | A | 884 | 35.193 | 81.721 | −4.570 | 1.00 | 13.79 | A |
| ATOM | 6930 | N | ASN | A | 885 | 33.095 | 82.024 | −5.329 | 1.00 | 12.72 | A |
| ATOM | 6931 | CA | ASN | A | 885 | 33.404 | 81.620 | −6.695 | 1.00 | 12.83 | A |
| ATOM | 6932 | CB | ASN | A | 885 | 32.163 | 81.746 | −7.579 | 1.00 | 12.72 | A |
| ATOM | 6933 | CG | ASN | A | 885 | 30.973 | 81.001 | −7.024 | 1.00 | 12.80 | A |
| ATOM | 6934 | OD1 | ASN | A | 885 | 30.700 | 79.860 | −7.403 | 1.00 | 13.90 | A |
| ATOM | 6935 | ND2 | ASN | A | 885 | 30.257 | 81.640 | −6.109 | 1.00 | 12.56 | A |
| ATOM | 6936 | C | ASN | A | 885 | 34.499 | 82.493 | −7.289 | 1.00 | 12.83 | A |
| ATOM | 6937 | O | ASN | A | 885 | 34.643 | 83.666 | −6.936 | 1.00 | 12.92 | A |
| ATOM | 6938 | N | LYS | A | 886 | 35.265 | 81.910 | −8.202 | 1.00 | 13.20 | A |
| ATOM | 6939 | CA | LYS | A | 886 | 36.329 | 82.624 | −8.887 | 1.00 | 13.92 | A |
| ATOM | 6940 | CB | LYS | A | 886 | 37.657 | 82.454 | −8.140 | 1.00 | 16.49 | A |
| ATOM | 6941 | CG | LYS | A | 886 | 38.096 | 81.015 | −7.938 | 1.00 | 17.99 | A |
| ATOM | 6942 | CD | LYS | A | 886 | 39.235 | 80.913 | −6.924 | 1.00 | 20.54 | A |
| ATOM | 6943 | CE | LYS | A | 886 | 40.472 | 81.669 | −7.386 | 1.00 | 21.34 | A |
| ATOM | 6944 | NZ | LYS | A | 886 | 41.570 | 81.615 | −6.383 | 1.00 | 22.47 | A |
| ATOM | 6945 | C | LYS | A | 886 | 36.420 | 82.055 | −10.295 | 1.00 | 12.96 | A |
| ATOM | 6946 | O | LYS | A | 886 | 36.052 | 80.903 | −10.534 | 1.00 | 12.96 | A |
| ATOM | 6947 | N | PRO | A | 887 | 36.890 | 82.859 | −11.256 | 1.00 | 12.00 | A |
| ATOM | 6948 | CD | PRO | A | 887 | 37.275 | 84.279 | −11.160 | 1.00 | 12.17 | A |
| ATOM | 6949 | CA | PRO | A | 887 | 37.002 | 82.378 | −12.633 | 1.00 | 10.97 | A |
| ATOM | 6950 | CB | PRO | A | 887 | 37.687 | 83.537 | −13.348 | 1.00 | 11.84 | A |
| ATOM | 6951 | CG | PRO | A | 887 | 37.179 | 84.731 | −12.593 | 1.00 | 12.42 | A |
| ATOM | 6952 | C | PRO | A | 887 | 37.805 | 81.087 | −12.734 | 1.00 | 10.67 | A |
| ATOM | 6953 | O | PRO | A | 887 | 38.866 | 80.951 | −12.129 | 1.00 | 11.60 | A |
| ATOM | 6954 | N | VAL | A | 888 | 37.282 | 80.137 | −13.498 | 1.00 | 9.48 | A |
| ATOM | 6955 | CA | VAL | A | 888 | 37.955 | 78.867 | −13.692 | 1.00 | 8.96 | A |
| ATOM | 6956 | CB | VAL | A | 888 | 37.335 | 77.755 | −12.797 | 1.00 | 9.00 | A |
| ATOM | 6957 | CG1 | VAL | A | 888 | 35.836 | 77.648 | −13.048 | 1.00 | 9.81 | A |
| ATOM | 6958 | CG2 | VAL | A | 888 | 38.021 | 76.424 | −13.064 | 1.00 | 9.13 | A |
| ATOM | 6959 | C | VAL | A | 888 | 37.852 | 78.466 | −15.153 | 1.00 | 8.88 | A |
| ATOM | 6960 | O | VAL | A | 888 | 36.823 | 78.697 | −15.802 | 1.00 | 8.91 | A |
| ATOM | 6961 | N | LEU | A | 889 | 38.928 | 77.890 | −15.677 | 1.00 | 7.75 | A |
| ATOM | 6962 | CA | LEU | A | 889 | 38.935 | 77.435 | −17.058 | 1.00 | 8.80 | A |
| ATOM | 6963 | CB | LEU | A | 889 | 40.257 | 77.794 | −17.745 | 1.00 | 9.25 | A |
| ATOM | 6964 | CG | LEU | A | 889 | 40.336 | 77.379 | −19.223 | 1.00 | 9.85 | A |
| ATOM | 6965 | CD1 | LEU | A | 889 | 39.386 | 78.245 | −20.050 | 1.00 | 11.06 | A |
| ATOM | 6966 | CD2 | LEU | A | 889 | 41.766 | 77.529 | −19.728 | 1.00 | 12.10 | A |
| ATOM | 6967 | C | LEU | A | 889 | 38.731 | 75.924 | −17.113 | 1.00 | 8.95 | A |
| ATOM | 6968 | O | LEU | A | 889 | 39.648 | 75.150 | −16.820 | 1.00 | 9.54 | A |
| ATOM | 6969 | N | HIS | A | 890 | 37.519 | 75.508 | −17.462 | 1.00 | 7.95 | A |
| ATOM | 6970 | CA | HIS | A | 890 | 37.214 | 74.086 | −17.580 | 1.00 | 8.10 | A |
| ATOM | 6971 | CB | HIS | A | 890 | 35.725 | 73.824 | −17.360 | 1.00 | 7.90 | A |
| ATOM | 6972 | CG | HIS | A | 890 | 35.281 | 73.998 | −15.944 | 1.00 | 8.99 | A |
| ATOM | 6973 | CD2 | HIS | A | 890 | 34.112 | 74.441 | −15.426 | 1.00 | 9.25 | A |
| ATOM | 6974 | ND1 | HIS | A | 890 | 36.066 | 73.637 | −14.870 | 1.00 | 9.76 | A |
| ATOM | 6975 | CE1 | HIS | A | 890 | 35.399 | 73.851 | −13.751 | 1.00 | 9.66 | A |
| ATOM | 6976 | NE2 | HIS | A | 890 | 34.210 | 74.339 | −14.060 | 1.00 | 10.10 | A |
| ATOM | 6977 | C | HIS | A | 890 | 37.584 | 73.618 | −18.977 | 1.00 | 7.85 | A |
| ATOM | 6978 | O | HIS | A | 890 | 37.361 | 74.335 | −19.952 | 1.00 | 8.19 | A |
| ATOM | 6979 | N | ILE | A | 891 | 38.136 | 72.415 | −19.079 | 1.00 | 7.06 | A |
| ATOM | 6980 | CA | ILE | A | 891 | 38.519 | 71.885 | −20.376 | 1.00 | 7.02 | A |
| ATOM | 6981 | CB | ILE | A | 891 | 40.055 | 71.799 | −20.507 | 1.00 | 6.50 | A |
| ATOM | 6982 | CG2 | ILE | A | 891 | 40.666 | 73.181 | −20.267 | 1.00 | 9.58 | A |
| ATOM | 6983 | CG1 | ILE | A | 891 | 40.627 | 70.806 | −19.492 | 1.00 | 7.14 | A |
| ATOM | 6984 | CD1 | ILE | A | 891 | 42.131 | 70.611 | −19.644 | 1.00 | 7.48 | A |
| ATOM | 6985 | C | ILE | A | 891 | 37.886 | 70.523 | −20.633 | 1.00 | 6.87 | A |
| ATOM | 6986 | O | ILE | A | 891 | 37.640 | 69.751 | −19.699 | 1.00 | 6.90 | A |
| ATOM | 6987 | N | TYR | A | 892 | 37.619 | 70.244 | −21.905 | 1.00 | 6.24 | A |
| ATOM | 6988 | CA | TYR | A | 892 | 36.982 | 68.996 | −22.326 | 1.00 | 6.85 | A |
| ATOM | 6989 | CB | TYR | A | 892 | 35.454 | 69.156 | −22.391 | 1.00 | 6.74 | A |
| ATOM | 6990 | CG | TYR | A | 892 | 34.795 | 69.841 | −21.224 | 1.00 | 6.68 | A |
| ATOM | 6991 | CD1 | TYR | A | 892 | 34.759 | 71.233 | −21.128 | 1.00 | 7.35 | A |
| ATOM | 6992 | CE1 | TYR | A | 892 | 34.141 | 71.860 | −20.048 | 1.00 | 6.58 | A |
| ATOM | 6993 | CD2 | TYR | A | 892 | 34.200 | 69.091 | −20.214 | 1.00 | 7.90 | A |
| ATOM | 6994 | CE2 | TYR | A | 892 | 33.589 | 69.699 | −19.136 | 1.00 | 8.12 | A |
| ATOM | 6995 | CZ | TYR | A | 892 | 33.560 | 71.080 | −19.054 | 1.00 | 7.15 | A |
| ATOM | 6996 | OH | TYR | A | 892 | 32.959 | 71.661 | −17.970 | 1.00 | 8.40 | A |
| ATOM | 6997 | C | TYR | A | 892 | 37.395 | 68.586 | −23.731 | 1.00 | 7.49 | A |
| ATOM | 6998 | O | TYR | A | 892 | 38.014 | 69.349 | −24.463 | 1.00 | 7.08 | A |
| ATOM | 6999 | N | ARG | A | 893 | 37.038 | 67.355 | −24.091 | 1.00 | 7.23 | A |
| ATOM | 7000 | CA | ARG | A | 893 | 37.233 | 66.847 | −25.449 | 1.00 | 7.84 | A |
| ATOM | 7001 | CB | ARG | A | 893 | 38.323 | 65.771 | −25.534 | 1.00 | 8.25 | A |
| ATOM | 7002 | CG | ARG | A | 893 | 39.761 | 66.292 | −25.460 | 1.00 | 9.07 | A |
| ATOM | 7003 | CD | ARG | A | 893 | 40.094 | 67.317 | −26.556 | 1.00 | 8.68 | A |
| ATOM | 7004 | NE | ARG | A | 893 | 40.144 | 66.752 | −27.905 | 1.00 | 10.22 | A |
| ATOM | 7005 | CZ | ARG | A | 893 | 41.105 | 65.951 | −28.358 | 1.00 | 9.15 | A |
| ATOM | 7006 | NH1 | ARG | A | 893 | 42.118 | 65.605 | −27.570 | 1.00 | 11.22 | A |
| ATOM | 7007 | NH2 | ARG | A | 893 | 41.058 | 65.496 | −29.603 | 1.00 | 12.32 | A |
| ATOM | 7008 | C | ARG | A | 893 | 35.867 | 66.244 | −25.776 | 1.00 | 8.20 | A |
| ATOM | 7009 | O | ARG | A | 893 | 35.258 | 65.580 | −24.933 | 1.00 | 8.49 | A |
| ATOM | 7010 | N | LEU | A | 894 | 35.374 | 66.502 | −26.984 | 1.00 | 8.73 | A |
| ATOM | 7011 | CA | LEU | A | 894 | 34.071 | 65.995 | −27.402 | 1.00 | 9.10 | A |
| ATOM | 7012 | CB | LEU | A | 894 | 33.175 | 67.152 | −27.847 | 1.00 | 10.48 | A |
| ATOM | 7013 | CG | LEU | A | 894 | 31.735 | 66.780 | −28.215 | 1.00 | 11.47 | A |
| ATOM | 7014 | CD1 | LEU | A | 894 | 31.013 | 66.238 | −26.992 | 1.00 | 12.94 | A |
| ATOM | 7015 | CD2 | LEU | A | 894 | 31.013 | 68.001 | −28.757 | 1.00 | 12.24 | A |
| ATOM | 7016 | C | LEU | A | 894 | 34.275 | 65.019 | −28.550 | 1.00 | 8.70 | A |
| ATOM | 7017 | O | LEU | A | 894 | 34.684 | 65.410 | −29.642 | 1.00 | 9.47 | A |
| ATOM | 7018 | N | VAL | A | 895 | 33.977 | 63.749 | −28.296 | 1.00 | 8.58 | A |
| ATOM | 7019 | CA | VAL | A | 895 | 34.173 | 62.699 | −29.284 | 1.00 | 8.99 | A |
| ATOM | 7020 | CB | VAL | A | 895 | 35.094 | 61.598 | −28.705 | 1.00 | 9.22 | A |
| ATOM | 7021 | CG1 | VAL | A | 895 | 35.461 | 60.598 | −29.786 | 1.00 | 10.34 | A |
| ATOM | 7022 | CG2 | VAL | A | 895 | 36.340 | 62.229 | −28.102 | 1.00 | 9.86 | A |
| ATOM | 7023 | C | VAL | A | 895 | 32.892 | 62.033 | −29.790 | 1.00 | 10.00 | A |
| ATOM | 7024 | O | VAL | A | 895 | 32.234 | 61.289 | −29.057 | 1.00 | 10.19 | A |
| ATOM | 7025 | N | LEU | A | 896 | 32.546 | 62.310 | −31.045 | 1.00 | 10.27 | A |
| ATOM | 7026 | CA | LEU | A | 896 | 31.376 | 61.699 | −31.677 | 1.00 | 10.89 | A |
| ATOM | 7027 | CB | LEU | A | 896 | 30.666 | 62.688 | −32.607 | 1.00 | 11.43 | A |
| ATOM | 7028 | CG | LEU | A | 896 | 29.525 | 62.085 | −33.436 | 1.00 | 12.15 | A |
| ATOM | 7029 | CD1 | LEU | A | 896 | 28.370 | 61.713 | −32.528 | 1.00 | 12.66 | A |
| ATOM | 7030 | CD2 | LEU | A | 896 | 29.061 | 63.089 | −34.479 | 1.00 | 12.63 | A |
| ATOM | 7031 | C | LEU | A | 896 | 31.938 | 60.545 | −32.501 | 1.00 | 11.72 | A |
| ATOM | 7032 | O | LEU | A | 896 | 32.880 | 60.731 | −33.269 | 1.00 | 11.16 | A |
| ATOM | 7033 | N | GLU | A | 897 | 31.371 | 59.354 | −32.339 | 1.00 | 11.32 | A |
| ATOM | 7034 | CA | GLU | A | 897 | 31.869 | 58.189 | −33.065 | 1.00 | 13.03 | A |
| ATOM | 7035 | CB | GLU | A | 897 | 32.844 | 57.397 | −32.192 | 1.00 | 14.50 | A |
| ATOM | 7036 | CG | GLU | A | 897 | 34.027 | 58.161 | −31.656 | 1.00 | 16.78 | A |
| ATOM | 7037 | CD | GLU | A | 897 | 34.778 | 57.365 | −30.601 | 1.00 | 16.72 | A |
| ATOM | 7038 | OE1 | GLU | A | 897 | 34.184 | 57.079 | −29.539 | 1.00 | 18.39 | A |
| ATOM | 7039 | OE2 | GLU | A | 897 | 35.955 | 57.027 | −30.838 | 1.00 | 19.39 | A |
| ATOM | 7040 | C | GLU | A | 897 | 30.770 | 57.228 | −33.478 | 1.00 | 12.78 | A |
| ATOM | 7041 | O | GLU | A | 897 | 29.699 | 57.192 | −32.873 | 1.00 | 12.49 | A |
| ATOM | 7042 | N | LYS | A | 898 | 31.056 | 56.441 | −34.509 | 1.00 | 14.12 | A |
| ATOM | 7043 | CA | LYS | A | 898 | 30.134 | 55.415 | −34.967 | 1.00 | 15.19 | A |
| ATOM | 7044 | CB | LYS | A | 898 | 30.283 | 55.175 | −36.470 | 1.00 | 16.61 | A |
| ATOM | 7045 | CG | LYS | A | 898 | 29.981 | 56.394 | −37.328 | 1.00 | 19.37 | A |
| ATOM | 7046 | CD | LYS | A | 898 | 28.614 | 56.986 | −37.004 | 1.00 | 21.68 | A |
| ATOM | 7047 | CE | LYS | A | 898 | 27.500 | 55.967 | −37.189 | 1.00 | 23.36 | A |
| ATOM | 7048 | NZ | LYS | A | 898 | 26.169 | 56.541 | −36.865 | 1.00 | 24.72 | A |
| ATOM | 7049 | C | LYS | A | 898 | 30.596 | 54.186 | −34.192 | 1.00 | 15.30 | A |
| ATOM | 7050 | O | LYS | A | 898 | 31.791 | 53.885 | −34.154 | 1.00 | 17.00 | A |
| ATOM | 7051 | N | VAL | A | 899 | 29.666 | 53.482 | −33.562 | 1.00 | 14.35 | A |
| ATOM | 7052 | CA | VAL | A | 899 | 30.038 | 52.318 | −32.773 | 1.00 | 14.25 | A |
| ATOM | 7053 | CB | VAL | A | 899 | 29.804 | 52.590 | −31.270 | 1.00 | 14.32 | A |
| ATOM | 7054 | CG1 | VAL | A | 899 | 30.809 | 53.622 | −30.771 | 1.00 | 15.02 | A |
| ATOM | 7055 | CG2 | VAL | A | 899 | 28.386 | 53.096 | −31.044 | 1.00 | 13.91 | A |
| ATOM | 7056 | C | VAL | A | 899 | 29.305 | 51.043 | −33.173 | 1.00 | 14.59 | A |
| ATOM | 7057 | O | VAL | A | 899 | 29.321 | 50.056 | −32.441 | 1.00 | 14.07 | A |
| ATOM | 7058 | N | ASN | A | 900 | 28.675 | 51.060 | −34.341 | 1.00 | 15.09 | A |
| ATOM | 7059 | CA | ASN | A | 900 | 27.941 | 49.893 | −34.813 | 1.00 | 16.30 | A |
| ATOM | 7060 | CB | ASN | A | 900 | 27.189 | 50.229 | −36.105 | 1.00 | 17.35 | A |
| ATOM | 7061 | CG | ASN | A | 900 | 28.098 | 50.780 | −37.182 | 1.00 | 18.71 | A |
| ATOM | 7062 | OD1 | ASN | A | 900 | 28.719 | 51.830 | −37.011 | 1.00 | 21.26 | A |
| ATOM | 7063 | ND2 | ASN | A | 900 | 28.182 | 50.073 | −38.304 | 1.00 | 20.56 | A |
| ATOM | 7064 | C | ASN | A | 900 | 28.841 | 48.677 | −35.041 | 1.00 | 16.24 | A |
| ATOM | 7065 | O | ASN | A | 900 | 28.366 | 47.541 | −35.023 | 1.00 | 16.99 | A |
| ATOM | 7066 | N | ASN | A | 901 | 30.135 | 48.907 | −35.243 | 1.00 | 15.40 | A |
| ATOM | 7067 | CA | ASN | A | 901 | 31.073 | 47.810 | −35.477 | 1.00 | 15.76 | A |
| ATOM | 7068 | CB | ASN | A | 901 | 32.054 | 48.177 | −36.592 | 1.00 | 17.95 | A |
| ATOM | 7069 | CG | ASN | A | 901 | 31.405 | 48.194 | −37.955 | 1.00 | 20.12 | A |
| ATOM | 7070 | OD1 | ASN | A | 901 | 30.737 | 47.238 | −38.345 | 1.00 | 22.38 | A |
| ATOM | 7071 | ND2 | ASN | A | 901 | 31.606 | 49.280 | −38.694 | 1.00 | 21.62 | A |
| ATOM | 7072 | C | ASN | A | 901 | 31.867 | 47.398 | −34.246 | 1.00 | 15.11 | A |
| ATOM | 7073 | O | ASN | A | 901 | 32.655 | 46.454 | −34.300 | 1.00 | 15.56 | A |
| ATOM | 7074 | N | CYS | A | 902 | 31.667 | 48.100 | −33.138 | 1.00 | 14.34 | A |
| ATOM | 7075 | CA | CYS | A | 902 | 32.398 | 47.784 | −31.916 | 1.00 | 14.62 | A |
| ATOM | 7076 | C | CYS | A | 902 | 31.829 | 46.594 | −31.165 | 1.00 | 13.93 | A |
| ATOM | 7077 | O | CYS | A | 902 | 30.616 | 46.407 | −31.101 | 1.00 | 14.93 | A |
| ATOM | 7078 | CB | CYS | A | 902 | 32.394 | 48.968 | −30.954 | 1.00 | 16.27 | A |
| ATOM | 7079 | SG | CYS | A | 902 | 33.091 | 50.528 | −31.570 | 1.00 | 18.03 | A |
| ATOM | 7080 | N | VAL | A | 903 | 32.717 | 45.798 | −30.584 | 1.00 | 13.19 | A |
| ATOM | 7081 | CA | VAL | A | 903 | 32.294 | 44.656 | −29.789 | 1.00 | 13.49 | A |
| ATOM | 7082 | CB | VAL | A | 903 | 33.403 | 43.584 | −29.706 | 1.00 | 12.99 | A |
| ATOM | 7083 | CG1 | VAL | A | 903 | 32.985 | 42.466 | −28.755 | 1.00 | 14.69 | A |
| ATOM | 7084 | CG2 | VAL | A | 903 | 33.674 | 43.021 | −31.095 | 1.00 | 14.28 | A |
| ATOM | 7085 | C | VAL | A | 903 | 32.024 | 45.228 | −28.400 | 1.00 | 13.60 | A |
| ATOM | 7086 | O | VAL | A | 903 | 32.952 | 45.524 | −27.646 | 1.00 | 15.18 | A |
| ATOM | 7087 | N | ARG | A | 904 | 30.749 | 45.409 | −28.078 | 1.00 | 13.74 | A |
| ATOM | 7088 | CA | ARG | A | 904 | 30.361 | 45.971 | −26.792 | 1.00 | 13.73 | A |
| ATOM | 7089 | CB | ARG | A | 904 | 29.311 | 47.066 | −27.001 | 1.00 | 14.82 | A |
| ATOM | 7090 | CG | ARG | A | 904 | 29.874 | 48.318 | −27.644 | 1.00 | 15.86 | A |
| ATOM | 7091 | CD | ARG | A | 904 | 28.816 | 49.366 | −27.921 | 1.00 | 17.64 | A |
| ATOM | 7092 | NE | ARG | A | 904 | 28.022 | 49.029 | −29.095 | 1.00 | 18.11 | A |
| ATOM | 7093 | CZ | ARG | A | 904 | 27.057 | 49.799 | −29.589 | 1.00 | 18.82 | A |
| ATOM | 7094 | NH1 | ARG | A | 904 | 26.763 | 50.955 | −29.007 | 1.00 | 19.22 | A |
| ATOM | 7095 | NH2 | ARG | A | 904 | 26.390 | 49.414 | −30.668 | 1.00 | 19.04 | A |
| ATOM | 7096 | C | ARG | A | 904 | 29.814 | 44.929 | −25.829 | 1.00 | 12.99 | A |
| ATOM | 7097 | O | ARG | A | 904 | 29.454 | 43.817 | −26.232 | 1.00 | 13.24 | A |
| ATOM | 7098 | N | PRO | A | 905 | 29.758 | 45.273 | −24.533 | 1.00 | 12.45 | A |
| ATOM | 7099 | CD | PRO | A | 905 | 30.305 | 46.482 | −23.890 | 1.00 | 12.91 | A |
| ATOM | 7100 | CA | PRO | A | 905 | 29.236 | 44.339 | −23.533 | 1.00 | 12.91 | A |
| ATOM | 7101 | CB | PRO | A | 905 | 29.412 | 45.099 | −22.220 | 1.00 | 12.51 | A |
| ATOM | 7102 | CG | PRO | A | 905 | 30.581 | 45.995 | −22.491 | 1.00 | 12.54 | A |
| ATOM | 7103 | C | PRO | A | 905 | 27.764 | 44.086 | −23.835 | 1.00 | 13.36 | A |
| ATOM | 7104 | O | PRO | A | 905 | 27.128 | 44.879 | −24.531 | 1.00 | 13.15 | A |
| ATOM | 7105 | N | SER | A | 906 | 27.224 | 42.987 | −23.318 | 1.00 | 14.23 | A |
| ATOM | 7106 | CA | SER | A | 906 | 25.817 | 42.679 | −23.540 | 1.00 | 16.23 | A |
| ATOM | 7107 | CB | SER | A | 906 | 25.479 | 41.285 | −23.014 | 1.00 | 17.70 | A |
| ATOM | 7108 | OG | SER | A | 906 | 25.354 | 41.297 | −21.605 | 1.00 | 19.81 | A |
| ATOM | 7109 | C | SER | A | 906 | 24.969 | 43.712 | −22.808 | 1.00 | 16.91 | A |
| ATOM | 7110 | O | SER | A | 906 | 25.478 | 44.485 | −21.993 | 1.00 | 16.27 | A |
| ATOM | 7111 | N | LYS | A | 907 | 23.673 | 43.709 | −23.093 | 1.00 | 18.02 | A |
| ATOM | 7112 | CA | LYS | A | 907 | 22.745 | 44.652 | −22.484 | 1.00 | 19.95 | A |
| ATOM | 7113 | CB | LYS | A | 907 | 21.350 | 44.464 | −23.085 | 1.00 | 22.55 | A |
| ATOM | 7114 | CG | LYS | A | 907 | 21.266 | 44.784 | −24.569 | 1.00 | 26.38 | A |
| ATOM | 7115 | CD | LYS | A | 907 | 19.889 | 44.452 | −25.136 | 1.00 | 28.71 | A |
| ATOM | 7116 | CE | LYS | A | 907 | 18.782 | 45.192 | −24.396 | 1.00 | 30.39 | A |
| ATOM | 7117 | NZ | LYS | A | 907 | 18.936 | 46.670 | −24.487 | 1.00 | 31.53 | A |
| ATOM | 7118 | C | LYS | A | 907 | 22.663 | 44.531 | −20.967 | 1.00 | 18.84 | A |
| ATOM | 7119 | O | LYS | A | 907 | 22.262 | 45.476 | −20.287 | 1.00 | 20.33 | A |
| ATOM | 7120 | N | LEU | A | 908 | 23.047 | 43.376 | −20.434 | 1.00 | 17.98 | A |
| ATOM | 7121 | CA | LEU | A | 908 | 22.982 | 43.159 | −18.993 | 1.00 | 17.30 | A |
| ATOM | 7122 | CB | LEU | A | 908 | 22.520 | 41.726 | −18.704 | 1.00 | 18.78 | A |
| ATOM | 7123 | CG | LEU | A | 908 | 21.150 | 41.355 | −19.284 | 1.00 | 19.88 | A |
| ATOM | 7124 | CD1 | LEU | A | 908 | 20.846 | 39.898 | −18.980 | 1.00 | 19.94 | A |
| ATOM | 7125 | CD2 | LEU | A | 908 | 20.070 | 42.263 | −18.701 | 1.00 | 20.79 | A |
| ATOM | 7126 | C | LEU | A | 908 | 24.289 | 43.439 | −18.255 | 1.00 | 15.70 | A |
| ATOM | 7127 | O | LEU | A | 908 | 24.341 | 43.357 | −17.028 | 1.00 | 15.90 | A |
| ATOM | 7128 | N | HIS | A | 909 | 25.340 | 43.779 | −18.992 | 1.00 | 14.37 | A |
| ATOM | 7129 | CA | HIS | A | 909 | 26.628 | 44.065 | −18.365 | 1.00 | 12.69 | A |
| ATOM | 7130 | CB | HIS | A | 909 | 27.737 | 44.014 | −19.415 | 1.00 | 12.14 | A |
| ATOM | 7131 | CG | HIS | A | 909 | 29.101 | 43.793 | −18.842 | 1.00 | 10.96 | A |
| ATOM | 7132 | CD2 | HIS | A | 909 | 29.934 | 42.728 | −18.912 | 1.00 | 11.76 | A |
| ATOM | 7133 | ND1 | HIS | A | 909 | 29.752 | 44.739 | −18.079 | 1.00 | 10.65 | A |
| ATOM | 7134 | CE1 | HIS | A | 909 | 30.928 | 44.265 | −17.706 | 1.00 | 11.60 | A |
| ATOM | 7135 | NE2 | HIS | A | 909 | 31.063 | 43.047 | −18.199 | 1.00 | 11.85 | A |
| ATOM | 7136 | C | HIS | A | 909 | 26.572 | 45.446 | −17.704 | 1.00 | 11.79 | A |
| ATOM | 7137 | O | HIS | A | 909 | 26.102 | 46.410 | −18.304 | 1.00 | 12.37 | A |
| ATOM | 7138 | N | PRO | A | 910 | 27.053 | 45.558 | −16.455 | 1.00 | 10.34 | A |
| ATOM | 7139 | CD | PRO | A | 910 | 27.391 | 44.457 | −15.540 | 1.00 | 11.26 | A |
| ATOM | 7140 | CA | PRO | A | 910 | 27.037 | 46.837 | −15.734 | 1.00 | 10.21 | A |
| ATOM | 7141 | CB | PRO | A | 910 | 27.184 | 46.421 | −14.268 | 1.00 | 10.93 | A |
| ATOM | 7142 | CG | PRO | A | 910 | 26.839 | 44.971 | −14.242 | 1.00 | 11.48 | A |
| ATOM | 7143 | C | PRO | A | 910 | 28.118 | 47.848 | −16.116 | 1.00 | 9.86 | A |
| ATOM | 7144 | O | PRO | A | 910 | 28.100 | 48.980 | −15.631 | 1.00 | 9.94 | A |
| ATOM | 7145 | N | ALA | A | 911 | 29.061 | 47.442 | −16.957 | 1.00 | 9.55 | A |
| ATOM | 7146 | CA | ALA | A | 911 | 30.138 | 48.342 | −17.342 | 1.00 | 9.81 | A |
| ATOM | 7147 | CB | ALA | A | 911 | 31.482 | 47.684 | −17.070 | 1.00 | 9.39 | A |
| ATOM | 7148 | C | ALA | A | 911 | 30.088 | 48.800 | −18.786 | 1.00 | 9.68 | A |
| ATOM | 7149 | O | ALA | A | 911 | 29.354 | 48.253 | −19.614 | 1.00 | 10.29 | A |
| ATOM | 7150 | N | GLY | A | 912 | 30.893 | 49.818 | −19.064 | 1.00 | 9.71 | A |
| ATOM | 7151 | CA | GLY | A | 912 | 31.022 | 50.367 | −20.397 | 1.00 | 9.40 | A |
| ATOM | 7152 | C | GLY | A | 912 | 32.504 | 50.627 | −20.589 | 1.00 | 9.56 | A |
| ATOM | 7153 | O | GLY | A | 912 | 33.244 | 50.718 | −19.607 | 1.00 | 9.93 | A |
| ATOM | 7154 | N | TYR | A | 913 | 32.943 | 50.749 | −21.838 | 1.00 | 9.13 | A |
| ATOM | 7155 | CA | TYR | A | 913 | 34.351 | 50.986 | −22.132 | 1.00 | 9.27 | A |
| ATOM | 7156 | CB | TYR | A | 913 | 35.042 | 49.682 | −22.547 | 1.00 | 8.76 | A |
| ATOM | 7157 | CG | TYR | A | 913 | 35.014 | 48.632 | −21.470 | 1.00 | 8.89 | A |
| ATOM | 7158 | CD1 | TYR | A | 913 | 34.036 | 47.641 | −21.460 | 1.00 | 10.18 | A |
| ATOM | 7159 | CE1 | TYR | A | 913 | 33.962 | 46.719 | −20.423 | 1.00 | 10.05 | A |
| ATOM | 7160 | CD2 | TYR | A | 913 | 35.923 | 48.674 | −20.418 | 1.00 | 9.62 | A |
| ATOM | 7161 | CE2 | TYR | A | 913 | 35.858 | 47.764 | −19.376 | 1.00 | 9.58 | A |
| ATOM | 7162 | CZ | TYR | A | 913 | 34.875 | 46.790 | −19.383 | 1.00 | 10.29 | A |
| ATOM | 7163 | OH | TYR | A | 913 | 34.789 | 45.897 | −18.341 | 1.00 | 10.44 | A |
| ATOM | 7164 | C | TYR | A | 913 | 34.548 | 52.013 | −23.230 | 1.00 | 9.23 | A |
| ATOM | 7165 | O | TYR | A | 913 | 33.759 | 52.093 | −24.173 | 1.00 | 9.94 | A |
| ATOM | 7166 | N | LEU | A | 914 | 35.617 | 52.790 | −23.101 | 1.00 | 9.01 | A |
| ATOM | 7167 | CA | LEU | A | 914 | 35.946 | 53.811 | −24.082 | 1.00 | 8.49 | A |
| ATOM | 7168 | CB | LEU | A | 914 | 36.951 | 54.809 | −23.507 | 1.00 | 8.20 | A |
| ATOM | 7169 | CG | LEU | A | 914 | 36.543 | 55.679 | −22.326 | 1.00 | 8.55 | A |
| ATOM | 7170 | CD1 | LEU | A | 914 | 37.659 | 56.679 | −22.057 | 1.00 | 8.59 | A |
| ATOM | 7171 | CD2 | LEU | A | 914 | 35.240 | 56.408 | −22.635 | 1.00 | 8.59 | A |
| ATOM | 7172 | C | LEU | A | 914 | 36.568 | 53.215 | −25.332 | 1.00 | 8.86 | A |
| ATOM | 7173 | O | LEU | A | 914 | 37.033 | 52.075 | −25.337 | 1.00 | 9.04 | A |
| ATOM | 7174 | N | THR | A | 915 | 36.570 | 54.021 | −26.387 | 1.00 | 9.49 | A |
| ATOM | 7175 | CA | THR | A | 915 | 37.187 | 53.656 | −27.647 | 1.00 | 10.01 | A |
| ATOM | 7176 | CB | THR | A | 915 | 36.498 | 54.345 | −28.831 | 1.00 | 11.05 | A |
| ATOM | 7177 | OG1 | THR | A | 915 | 36.474 | 55.758 | −28.590 | 1.00 | 12.54 | A |
| ATOM | 7178 | CG2 | THR | A | 915 | 35.080 | 53.833 | −29.011 | 1.00 | 12.14 | A |
| ATOM | 7179 | C | THR | A | 915 | 38.593 | 54.237 | −27.542 | 1.00 | 10.47 | A |
| ATOM | 7180 | O | THR | A | 915 | 38.879 | 55.037 | −26.641 | 1.00 | 10.28 | A |
| ATOM | 7181 | N | SER | A | 916 | 39.464 | 53.853 | −28.465 | 1.00 | 11.05 | A |
| ATOM | 7182 | CA | SER | A | 916 | 40.828 | 54.357 | −28.482 | 1.00 | 11.00 | A |
| ATOM | 7183 | CB | SER | A | 916 | 41.574 | 53.795 | −29.690 | 1.00 | 12.42 | A |
| ATOM | 7184 | OG | SER | A | 916 | 42.793 | 54.485 | −29.888 | 1.00 | 16.48 | A |
| ATOM | 7185 | C | SER | A | 916 | 40.857 | 55.881 | −28.539 | 1.00 | 10.98 | A |
| ATOM | 7186 | O | SER | A | 916 | 41.572 | 56.528 | −27.776 | 1.00 | 10.02 | A |
| ATOM | 7187 | N | ALA | A | 917 | 40.069 | 56.455 | −29.442 | 1.00 | 10.04 | A |
| ATOM | 7188 | CA | ALA | A | 917 | 40.040 | 57.903 | −29.593 | 1.00 | 9.89 | A |
| ATOM | 7189 | CB | ALA | A | 917 | 39.119 | 58.292 | −30.738 | 1.00 | 10.71 | A |
| ATOM | 7190 | C | ALA | A | 917 | 39.612 | 58.621 | −28.319 | 1.00 | 9.48 | A |
| ATOM | 7191 | O | ALA | A | 917 | 40.199 | 59.638 | −27.950 | 1.00 | 9.99 | A |
| ATOM | 7192 | N | ALA | A | 918 | 38.592 | 58.099 | −27.644 | 1.00 | 9.40 | A |
| ATOM | 7193 | CA | ALA | A | 918 | 38.110 | 58.736 | −26.420 | 1.00 | 8.86 | A |
| ATOM | 7194 | CB | ALA | A | 918 | 36.788 | 58.114 | −25.988 | 1.00 | 9.92 | A |
| ATOM | 7195 | C | ALA | A | 918 | 39.141 | 58.627 | −25.300 | 1.00 | 8.91 | A |
| ATOM | 7196 | O | ALA | A | 918 | 39.331 | 59.569 | −24.524 | 1.00 | 7.94 | A |
| ATOM | 7197 | N | HIS | A | 919 | 39.799 | 57.477 | −25.210 | 1.00 | 8.73 | A |
| ATOM | 7198 | CA | HIS | A | 919 | 40.818 | 57.279 | −24.189 | 1.00 | 8.75 | A |
| ATOM | 7199 | CB | HIS | A | 919 | 41.320 | 55.833 | −24.211 | 1.00 | 9.47 | A |
| ATOM | 7200 | CG | HIS | A | 919 | 42.438 | 55.570 | −23.249 | 1.00 | 10.68 | A |
| ATOM | 7201 | CD2 | HIS | A | 919 | 42.499 | 55.686 | −21.901 | 1.00 | 12.48 | A |
| ATOM | 7202 | ND1 | HIS | A | 919 | 43.689 | 55.158 | −23.656 | 1.00 | 13.04 | A |
| ATOM | 7203 | CE1 | HIS | A | 919 | 44.473 | 55.032 | −22.599 | 1.00 | 12.08 | A |
| ATOM | 7204 | NE2 | HIS | A | 919 | 43.776 | 55.346 | −21.523 | 1.00 | 13.28 | A |
| ATOM | 7205 | C | HIS | A | 919 | 41.979 | 58.239 | −24.430 | 1.00 | 8.59 | A |
| ATOM | 7206 | O | HIS | A | 919 | 42.459 | 58.892 | −23.503 | 1.00 | 8.31 | A |
| ATOM | 7207 | N | LYS | A | 920 | 42.429 | 58.341 | −25.675 | 1.00 | 7.96 | A |
| ATOM | 7208 | CA | LYS | A | 920 | 43.528 | 59.250 | −25.969 | 1.00 | 9.08 | A |
| ATOM | 7209 | CB | LYS | A | 920 | 44.009 | 59.077 | −27.411 | 1.00 | 10.76 | A |
| ATOM | 7210 | CG | LYS | A | 920 | 44.880 | 57.833 | −27.582 | 1.00 | 13.02 | A |
| ATOM | 7211 | CD | LYS | A | 920 | 45.559 | 57.777 | −28.938 | 1.00 | 14.64 | A |
| ATOM | 7212 | CE | LYS | A | 920 | 46.493 | 56.579 | −29.030 | 1.00 | 15.02 | A |
| ATOM | 7213 | NZ | LYS | A | 920 | 47.658 | 56.686 | −28.103 | 1.00 | 15.98 | A |
| ATOM | 7214 | C | LYS | A | 920 | 43.116 | 60.694 | −25.701 | 1.00 | 8.52 | A |
| ATOM | 7215 | O | LYS | A | 920 | 43.928 | 61.496 | −25.236 | 1.00 | 8.56 | A |
| ATOM | 7216 | N | ALA | A | 921 | 41.855 | 61.024 | −25.972 | 1.00 | 8.30 | A |
| ATOM | 7217 | CA | ALA | A | 921 | 41.372 | 62.380 | −25.729 | 1.00 | 8.01 | A |
| ATOM | 7218 | CB | ALA | A | 921 | 39.947 | 62.540 | −26.263 | 1.00 | 7.47 | A |
| ATOM | 7219 | C | ALA | A | 921 | 41.421 | 62.667 | −24.227 | 1.00 | 7.72 | A |
| ATOM | 7220 | O | ALA | A | 921 | 41.770 | 63.772 | −23.801 | 1.00 | 8.39 | A |
| ATOM | 7221 | N | SER | A | 922 | 41.076 | 61.670 | −23.417 | 1.00 | 7.17 | A |
| ATOM | 7222 | CA | SER | A | 922 | 41.120 | 61.854 | −21.969 | 1.00 | 7.27 | A |
| ATOM | 7223 | CB | SER | A | 922 | 40.549 | 60.627 | −21.251 | 1.00 | 7.70 | A |
| ATOM | 7224 | OG | SER | A | 922 | 40.649 | 60.781 | −19.841 | 1.00 | 7.96 | A |
| ATOM | 7225 | C | SER | A | 922 | 42.565 | 62.083 | −21.532 | 1.00 | 7.89 | A |
| ATOM | 7226 | O | SER | A | 922 | 42.839 | 62.944 | −20.698 | 1.00 | 8.87 | A |
| ATOM | 7227 | N | GLN | A | 923 | 43.494 | 61.321 | −22.104 | 1.00 | 8.46 | A |
| ATOM | 7228 | CA | GLN | A | 923 | 44.904 | 61.476 | −21.760 | 1.00 | 8.76 | A |
| ATOM | 7229 | CB | GLN | A | 923 | 45.744 | 60.379 | −22.423 | 1.00 | 8.95 | A |
| ATOM | 7230 | CG | GLN | A | 923 | 45.466 | 58.979 | −21.895 | 1.00 | 8.79 | A |
| ATOM | 7231 | CD | GLN | A | 923 | 46.396 | 57.936 | −22.495 | 1.00 | 10.09 | A |
| ATOM | 7232 | OE1 | GLN | A | 923 | 46.537 | 57.847 | −23.715 | 1.00 | 11.44 | A |
| ATOM | 7233 | NE2 | GLN | A | 923 | 47.028 | 57.133 | −21.635 | 1.00 | 9.11 | A |
| ATOM | 7234 | C | GLN | A | 923 | 45.435 | 62.849 | −22.163 | 1.00 | 9.21 | A |
| ATOM | 7235 | O | GLN | A | 923 | 46.318 | 63.396 | −21.501 | 1.00 | 9.10 | A |
| ATOM | 7236 | N | SER | A | 924 | 44.886 | 63.417 | −23.236 | 1.00 | 8.93 | A |
| ATOM | 7237 | CA | SER | A | 924 | 45.333 | 64.729 | −23.703 | 1.00 | 9.54 | A |
| ATOM | 7238 | CB | SER | A | 924 | 44.733 | 65.048 | −25.081 | 1.00 | 10.43 | A |
| ATOM | 7239 | OG | SER | A | 924 | 43.373 | 65.457 | −24.987 | 1.00 | 11.08 | A |
| ATOM | 7240 | C | SER | A | 924 | 44.941 | 65.819 | −22.709 | 1.00 | 9.66 | A |
| ATOM | 7241 | O | SER | A | 924 | 45.572 | 66.874 | −22.648 | 1.00 | 10.68 | A |
| ATOM | 7242 | N | LEU | A | 925 | 43.898 | 65.554 | −21.931 | 1.00 | 9.32 | A |
| ATOM | 7243 | CA | LEU | A | 925 | 43.412 | 66.508 | −20.941 | 1.00 | 8.62 | A |
| ATOM | 7244 | CB | LEU | A | 925 | 41.904 | 66.335 | −20.738 | 1.00 | 8.40 | A |
| ATOM | 7245 | CG | LEU | A | 925 | 40.991 | 66.609 | −21.938 | 1.00 | 7.47 | A |
| ATOM | 7246 | CD1 | LEU | A | 925 | 39.556 | 66.255 | −21.569 | 1.00 | 8.87 | A |
| ATOM | 7247 | CD2 | LEU | A | 925 | 41.094 | 68.077 | −22.349 | 1.00 | 8.87 | A |
| ATOM | 7248 | C | LEU | A | 925 | 44.106 | 66.351 | −19.593 | 1.00 | 9.08 | A |
| ATOM | 7249 | O | LEU | A | 925 | 44.532 | 67.333 | −18.984 | 1.00 | 9.69 | A |
| ATOM | 7250 | N | LEU | A | 926 | 44.236 | 65.110 | −19.141 | 1.00 | 8.72 | A |
| ATOM | 7251 | CA | LEU | A | 926 | 44.837 | 64.834 | −17.842 | 1.00 | 8.73 | A |
| ATOM | 7252 | CB | LEU | A | 926 | 44.287 | 63.518 | −17.291 | 1.00 | 9.50 | A |
| ATOM | 7253 | CG | LEU | A | 926 | 42.771 | 63.509 | −17.070 | 1.00 | 10.76 | A |
| ATOM | 7254 | CD1 | LEU | A | 926 | 42.339 | 62.149 | −16.537 | 1.00 | 11.48 | A |
| ATOM | 7255 | CD2 | LEU | A | 926 | 42.388 | 64.618 | −16.095 | 1.00 | 12.65 | A |
| ATOM | 7256 | C | LEU | A | 926 | 46.356 | 64.804 | −17.790 | 1.00 | 8.63 | A |
| ATOM | 7257 | O | LEU | A | 926 | 46.946 | 65.189 | −16.783 | 1.00 | 8.46 | A |
| ATOM | 7258 | N | ASP | A | 927 | 46.989 | 64.340 | −18.861 | 1.00 | 7.51 | A |
| ATOM | 7259 | CA | ASP | A | 927 | 48.444 | 64.262 | −18.891 | 1.00 | 8.28 | A |
| ATOM | 7260 | CB | ASP | A | 927 | 48.891 | 62.819 | −18.644 | 1.00 | 7.75 | A |
| ATOM | 7261 | CG | ASP | A | 927 | 48.570 | 62.350 | −17.234 | 1.00 | 8.29 | A |
| ATOM | 7262 | OD1 | ASP | A | 927 | 49.295 | 62.740 | −16.293 | 1.00 | 8.68 | A |
| ATOM | 7263 | OD2 | ASP | A | 927 | 47.580 | 61.609 | −17.067 | 1.00 | 9.56 | A |
| ATOM | 7264 | C | ASP | A | 927 | 49.008 | 64.787 | −20.204 | 1.00 | 8.04 | A |
| ATOM | 7265 | O | ASP | A | 927 | 49.595 | 64.049 | −20.995 | 1.00 | 7.83 | A |
| ATOM | 7266 | N | PRO | A | 928 | 48.830 | 66.088 | −20.452 | 1.00 | 8.56 | A |
| ATOM | 7267 | CD | PRO | A | 928 | 48.226 | 67.103 | −19.569 | 1.00 | 9.25 | A |
| ATOM | 7268 | CA | PRO | A | 928 | 49.334 | 66.697 | −21.683 | 1.00 | 8.71 | A |
| ATOM | 7269 | CB | PRO | A | 928 | 48.708 | 68.088 | −21.649 | 1.00 | 9.22 | A |
| ATOM | 7270 | CG | PRO | A | 928 | 48.717 | 68.403 | −20.190 | 1.00 | 10.86 | A |
| ATOM | 7271 | C | PRO | A | 928 | 50.855 | 66.770 | −21.665 | 1.00 | 8.77 | A |
| ATOM | 7272 | O | PRO | A | 928 | 51.492 | 66.449 | −20.661 | 1.00 | 9.15 | A |
| ATOM | 7273 | N | LEU | A | 929 | 51.446 | 67.178 | −22.778 | 1.00 | 8.33 | A |
| ATOM | 7274 | CA | LEU | A | 929 | 52.891 | 67.338 | −22.810 | 1.00 | 7.61 | A |
| ATOM | 7275 | CB | LEU | A | 929 | 53.368 | 67.690 | −24.221 | 1.00 | 8.02 | A |
| ATOM | 7276 | CG | LEU | A | 929 | 53.200 | 66.682 | −25.351 | 1.00 | 8.18 | A |
| ATOM | 7277 | CD1 | LEU | A | 929 | 53.766 | 67.292 | −26.632 | 1.00 | 8.11 | A |
| ATOM | 7278 | CD2 | LEU | A | 929 | 53.934 | 65.391 | −25.012 | 1.00 | 7.77 | A |
| ATOM | 7279 | C | LEU | A | 929 | 53.234 | 68.505 | −21.887 | 1.00 | 8.11 | A |
| ATOM | 7280 | O | LEU | A | 929 | 52.452 | 69.445 | −21.748 | 1.00 | 9.64 | A |
| ATOM | 7281 | N | ASP | A | 930 | 54.394 | 68.441 | −21.248 | 1.00 | 7.51 | A |
| ATOM | 7282 | CA | ASP | A | 930 | 54.841 | 69.534 | −20.390 | 1.00 | 7.84 | A |
| ATOM | 7283 | CB | ASP | A | 930 | 55.582 | 68.979 | −19.182 | 1.00 | 8.72 | A |
| ATOM | 7284 | CG | ASP | A | 930 | 54.742 | 68.005 | −18.411 | 1.00 | 9.94 | A |
| ATOM | 7285 | OD1 | ASP | A | 930 | 53.688 | 68.437 | −17.903 | 1.00 | 12.39 | A |
| ATOM | 7286 | OD2 | ASP | A | 930 | 55.121 | 66.818 | −18.334 | 1.00 | 9.76 | A |
| ATOM | 7287 | C | ASP | A | 930 | 55.767 | 70.394 | −21.237 | 1.00 | 8.70 | A |
| ATOM | 7288 | O | ASP | A | 930 | 56.531 | 69.870 | −22.055 | 1.00 | 9.26 | A |
| ATOM | 7289 | N | LYS | A | 931 | 55.702 | 71.705 | −21.047 | 1.00 | 8.39 | A |
| ATOM | 7290 | CA | LYS | A | 931 | 56.517 | 72.627 | −21.831 | 1.00 | 8.99 | A |
| ATOM | 7291 | CB | LYS | A | 931 | 55.605 | 73.584 | −22.605 | 1.00 | 10.48 | A |
| ATOM | 7292 | CG | LYS | A | 931 | 54.622 | 72.897 | −23.547 | 1.00 | 11.73 | A |
| ATOM | 7293 | CD | LYS | A | 931 | 53.708 | 73.912 | −24.221 | 1.00 | 14.44 | A |
| ATOM | 7294 | CE | LYS | A | 931 | 52.828 | 74.625 | −23.206 | 1.00 | 16.26 | A |
| ATOM | 7295 | NZ | LYS | A | 931 | 52.017 | 75.703 | −23.824 | 1.00 | 16.66 | A |
| ATOM | 7296 | C | LYS | A | 931 | 57.479 | 73.425 | −20.963 | 1.00 | 9.43 | A |
| ATOM | 7297 | O | LYS | A | 931 | 57.081 | 74.021 | −19.959 | 1.00 | 9.83 | A |
| ATOM | 7298 | N | PHE | A | 932 | 58.747 | 73.439 | −21.362 | 1.00 | 8.58 | A |
| ATOM | 7299 | CA | PHE | A | 932 | 59.781 | 74.160 | −20.633 | 1.00 | 8.60 | A |
| ATOM | 7300 | CB | PHE | A | 932 | 60.852 | 73.191 | −20.124 | 1.00 | 8.76 | A |
| ATOM | 7301 | CG | PHE | A | 932 | 60.326 | 72.107 | −19.229 | 1.00 | 9.77 | A |
| ATOM | 7302 | CD1 | PHE | A | 932 | 59.725 | 70.970 | −19.760 | 1.00 | 10.21 | A |
| ATOM | 7303 | CD2 | PHE | A | 932 | 60.450 | 72.216 | −17.849 | 1.00 | 10.48 | A |
| ATOM | 7304 | CE1 | PHE | A | 932 | 59.259 | 69.952 | −18.926 | 1.00 | 10.04 | A |
| ATOM | 7305 | CE2 | PHE | A | 932 | 59.986 | 71.205 | −17.004 | 1.00 | 10.11 | A |
| ATOM | 7306 | CZ | PHE | A | 932 | 59.391 | 70.073 | −17.545 | 1.00 | 10.61 | A |
| ATOM | 7307 | C | PHE | A | 932 | 60.451 | 75.194 | −21.530 | 1.00 | 9.09 | A |
| ATOM | 7308 | O | PHE | A | 932 | 60.755 | 74.910 | −22.684 | 1.00 | 9.97 | A |
| ATOM | 7309 | N | ILE | A | 933 | 60.674 | 76.389 | −20.993 | 1.00 | 9.13 | A |
| ATOM | 7310 | CA | ILE | A | 933 | 61.331 | 77.462 | −21.739 | 1.00 | 9.38 | A |
| ATOM | 7311 | CB | ILE | A | 933 | 60.531 | 78.783 | −21.666 | 1.00 | 9.33 | A |
| ATOM | 7312 | CG2 | ILE | A | 933 | 61.247 | 79.868 | −22.472 | 1.00 | 9.12 | A |
| ATOM | 7313 | CG1 | ILE | A | 933 | 59.108 | 78.578 | −22.194 | 1.00 | 9.64 | A |
| ATOM | 7314 | CD1 | ILE | A | 933 | 58.197 | 79.778 | −21.970 | 1.00 | 9.86 | A |
| ATOM | 7315 | C | ILE | A | 933 | 62.685 | 77.704 | −21.080 | 1.00 | 10.17 | A |
| ATOM | 7316 | O | ILE | A | 933 | 62.743 | 78.009 | −19.888 | 1.00 | 9.98 | A |
| ATOM | 7317 | N | PHE | A | 934 | 63.772 | 77.564 | −21.836 | 1.00 | 9.94 | A |
| ATOM | 7318 | CA | PHE | A | 934 | 65.094 | 77.789 | −21.258 | 1.00 | 11.19 | A |
| ATOM | 7319 | CB | PHE | A | 934 | 66.193 | 77.479 | −22.278 | 1.00 | 11.54 | A |
| ATOM | 7320 | CG | PHE | A | 934 | 67.570 | 77.438 | −21.682 | 1.00 | 12.50 | A |
| ATOM | 7321 | CD1 | PHE | A | 934 | 67.947 | 76.400 | −20.835 | 1.00 | 13.12 | A |
| ATOM | 7322 | CD2 | PHE | A | 934 | 68.486 | 78.449 | −21.949 | 1.00 | 13.26 | A |
| ATOM | 7323 | CE1 | PHE | A | 934 | 69.216 | 76.371 | −20.263 | 1.00 | 14.59 | A |
| ATOM | 7324 | CE2 | PHE | A | 934 | 69.754 | 78.432 | −21.385 | 1.00 | 14.88 | A |
| ATOM | 7325 | CZ | PHE | A | 934 | 70.122 | 77.392 | −20.539 | 1.00 | 15.40 | A |
| ATOM | 7326 | C | PHE | A | 934 | 65.182 | 79.247 | −20.800 | 1.00 | 12.24 | A |
| ATOM | 7327 | O | PHE | A | 934 | 64.892 | 80.165 | −21.563 | 1.00 | 11.22 | A |
| ATOM | 7328 | N | ALA | A | 935 | 65.586 | 79.453 | −19.552 | 1.00 | 13.19 | A |
| ATOM | 7329 | CA | ALA | A | 935 | 65.662 | 80.799 | −18.987 | 1.00 | 14.58 | A |
| ATOM | 7330 | CB | ALA | A | 935 | 65.718 | 80.710 | −17.466 | 1.00 | 15.65 | A |
| ATOM | 7331 | C | ALA | A | 935 | 66.808 | 81.675 | −19.492 | 1.00 | 16.26 | A |
| ATOM | 7332 | O | ALA | A | 935 | 66.590 | 82.825 | −19.891 | 1.00 | 17.74 | A |
| ATOM | 7333 | N | GLU | A | 936 | 68.022 | 81.136 | −19.467 | 1.00 | 16.39 | A |
| ATOM | 7334 | CA | GLU | A | 936 | 69.211 | 81.867 | −19.898 | 1.00 | 17.74 | A |
| ATOM | 7335 | CB | GLU | A | 936 | 70.467 | 81.103 | −19.468 | 1.00 | 20.01 | A |
| ATOM | 7336 | CG | GLU | A | 936 | 70.634 | 80.978 | −17.957 | 1.00 | 22.92 | A |
| ATOM | 7337 | CD | GLU | A | 936 | 71.701 | 79.965 | −17.566 | 1.00 | 24.26 | A |
| ATOM | 7338 | OE1 | GLU | A | 936 | 71.485 | 78.753 | −17.782 | 1.00 | 25.07 | A |
| ATOM | 7339 | OE2 | GLU | A | 936 | 72.757 | 80.381 | −17.048 | 1.00 | 25.95 | A |
| ATOM | 7340 | C | GLU | A | 936 | 69.246 | 82.112 | −21.404 | 1.00 | 17.36 | A |
| ATOM | 7341 | O | GLU | A | 936 | 68.448 | 81.555 | −22.154 | 1.00 | 16.51 | A |
| ATOM | 7342 | N | ASN | A | 937 | 70.182 | 82.945 | −21.847 | 1.00 | 17.29 | A |
| ATOM | 7343 | CA | ASN | A | 937 | 70.292 | 83.249 | −23.267 | 1.00 | 17.24 | A |
| ATOM | 7344 | CB | ASN | A | 937 | 71.132 | 84.511 | −23.481 | 1.00 | 17.86 | A |
| ATOM | 7345 | CG | ASN | A | 937 | 70.445 | 85.756 | −22.963 | 1.00 | 19.51 | A |
| ATOM | 7346 | OD1 | ASN | A | 937 | 69.224 | 85.891 | −23.065 | 1.00 | 19.45 | A |
| ATOM | 7347 | ND2 | ASN | A | 937 | 71.224 | 86.680 | −22.415 | 1.00 | 20.71 | A |
| ATOM | 7348 | C | ASN | A | 937 | 70.872 | 82.104 | −24.083 | 1.00 | 17.18 | A |
| ATOM | 7349 | O | ASN | A | 937 | 70.477 | 81.897 | −25.231 | 1.00 | 17.44 | A |
| ATOM | 7350 | N | GLU | A | 938 | 71.808 | 81.362 | −23.498 | 1.00 | 17.86 | A |
| ATOM | 7351 | CA | GLU | A | 938 | 72.420 | 80.242 | −24.200 | 1.00 | 18.98 | A |
| ATOM | 7352 | CB | GLU | A | 938 | 73.815 | 80.620 | −24.706 | 1.00 | 21.93 | A |
| ATOM | 7353 | CG | GLU | A | 938 | 74.555 | 79.461 | −25.356 | 1.00 | 25.69 | A |
| ATOM | 7354 | CD | GLU | A | 938 | 75.752 | 79.905 | −26.169 | 1.00 | 27.97 | A |
| ATOM | 7355 | OE1 | GLU | A | 938 | 76.636 | 80.591 | −25.613 | 1.00 | 29.35 | A |
| ATOM | 7356 | OE2 | GLU | A | 938 | 75.809 | 79.563 | −27.369 | 1.00 | 29.44 | A |
| ATOM | 7357 | C | GLU | A | 938 | 72.512 | 78.991 | −23.337 | 1.00 | 17.94 | A |
| ATOM | 7358 | O | GLU | A | 938 | 72.883 | 79.055 | −22.167 | 1.00 | 17.89 | A |
| ATOM | 7359 | N | TRP | A | 939 | 72.172 | 77.855 | −23.935 | 1.00 | 17.52 | A |
| ATOM | 7360 | CA | TRP | A | 939 | 72.205 | 76.566 | −23.254 | 1.00 | 16.76 | A |
| ATOM | 7361 | CB | TRP | A | 939 | 70.869 | 75.847 | −23.479 | 1.00 | 15.33 | A |
| ATOM | 7362 | CG | TRP | A | 939 | 70.741 | 74.489 | −22.850 | 1.00 | 13.31 | A |
| ATOM | 7363 | CD2 | TRP | A | 939 | 69.654 | 73.575 | −23.030 | 1.00 | 12.28 | A |
| ATOM | 7364 | CE2 | TRP | A | 939 | 69.933 | 72.431 | −22.250 | 1.00 | 12.13 | A |
| ATOM | 7365 | CE3 | TRP | A | 939 | 68.469 | 73.613 | −23.776 | 1.00 | 11.44 | A |
| ATOM | 7366 | CD1 | TRP | A | 939 | 71.615 | 73.882 | −21.991 | 1.00 | 13.47 | A |
| ATOM | 7367 | NE1 | TRP | A | 939 | 71.135 | 72.644 | −21.627 | 1.00 | 12.39 | A |
| ATOM | 7368 | CZ2 | TRP | A | 939 | 69.068 | 71.332 | −22.196 | 1.00 | 11.94 | A |
| ATOM | 7369 | CZ3 | TRP | A | 939 | 67.609 | 72.521 | −23.723 | 1.00 | 11.55 | A |
| ATOM | 7370 | CH2 | TRP | A | 939 | 67.915 | 71.395 | −22.938 | 1.00 | 12.10 | A |
| ATOM | 7371 | C | TRP | A | 939 | 73.362 | 75.744 | −23.814 | 1.00 | 17.70 | A |
| ATOM | 7372 | O | TRP | A | 939 | 73.224 | 75.072 | −24.836 | 1.00 | 18.81 | A |
| ATOM | 7373 | N | ILE | A | 940 | 74.509 | 75.815 | −23.144 | 1.00 | 18.72 | A |
| ATOM | 7374 | CA | ILE | A | 940 | 75.690 | 75.082 | −23.583 | 1.00 | 19.32 | A |
| ATOM | 7375 | CB | ILE | A | 940 | 76.958 | 75.609 | −22.879 | 1.00 | 20.59 | A |
| ATOM | 7376 | CG2 | ILE | A | 940 | 78.178 | 74.816 | −23.335 | 1.00 | 21.55 | A |
| ATOM | 7377 | CG1 | ILE | A | 940 | 77.143 | 77.095 | −23.194 | 1.00 | 21.42 | A |
| ATOM | 7378 | CD1 | ILE | A | 940 | 78.336 | 77.729 | −22.506 | 1.00 | 22.42 | A |
| ATOM | 7379 | C | ILE | A | 940 | 75.553 | 73.587 | −23.302 | 1.00 | 18.18 | A |
| ATOM | 7380 | O | ILE | A | 940 | 75.154 | 73.187 | −22.209 | 1.00 | 18.75 | A |
| ATOM | 7381 | N | GLY | A | 941 | 75.880 | 72.769 | −24.298 | 1.00 | 17.98 | A |
| ATOM | 7382 | CA | GLY | A | 941 | 75.795 | 71.327 | −24.131 | 1.00 | 16.47 | A |
| ATOM | 7383 | C | GLY | A | 941 | 74.396 | 70.764 | −24.300 | 1.00 | 16.11 | A |
| ATOM | 7384 | O | GLY | A | 941 | 74.140 | 69.610 | −23.953 | 1.00 | 14.65 | A |
| ATOM | 7385 | N | ALA | A | 942 | 73.490 | 71.572 | −24.839 | 1.00 | 15.44 | A |
| ATOM | 7386 | CA | ALA | A | 942 | 72.112 | 71.147 | −25.047 | 1.00 | 15.43 | A |
| ATOM | 7387 | CB | ALA | A | 942 | 71.295 | 72.300 | −25.618 | 1.00 | 15.09 | A |
| ATOM | 7388 | C | ALA | A | 942 | 72.002 | 69.934 | −25.967 | 1.00 | 15.86 | A |
| ATOM | 7389 | O | ALA | A | 942 | 72.715 | 69.829 | −26.964 | 1.00 | 16.24 | A |
| ATOM | 7390 | N | GLN | A | 943 | 71.098 | 69.022 | −25.619 | 1.00 | 15.50 | A |
| ATOM | 7391 | CA | GLN | A | 943 | 70.853 | 67.822 | −26.410 | 1.00 | 15.78 | A |
| ATOM | 7392 | CB | GLN | A | 943 | 71.049 | 66.571 | −25.553 | 1.00 | 16.41 | A |
| ATOM | 7393 | CG | GLN | A | 943 | 72.424 | 66.493 | −24.914 | 1.00 | 19.58 | A |
| ATOM | 7394 | CD | GLN | A | 943 | 72.627 | 65.219 | −24.123 | 1.00 | 20.93 | A |
| ATOM | 7395 | OE1 | GLN | A | 943 | 71.773 | 64.829 | −23.326 | 1.00 | 22.95 | A |
| ATOM | 7396 | NE2 | GLN | A | 943 | 73.764 | 64.565 | −24.333 | 1.00 | 22.03 | A |
| ATOM | 7397 | C | GLN | A | 943 | 69.422 | 67.898 | −26.934 | 1.00 | 15.16 | A |
| ATOM | 7398 | O | GLN | A | 943 | 68.554 | 68.504 | −26.302 | 1.00 | 14.93 | A |
| ATOM | 7399 | N | GLY | A | 944 | 69.171 | 67.277 | −28.080 | 1.00 | 14.52 | A |
| ATOM | 7400 | CA | GLY | A | 944 | 67.853 | 67.358 | −28.677 | 1.00 | 14.66 | A |
| ATOM | 7401 | C | GLY | A | 944 | 66.789 | 66.373 | −28.261 | 1.00 | 14.61 | A |
| ATOM | 7402 | O | GLY | A | 944 | 65.615 | 66.576 | −28.569 | 1.00 | 14.40 | A |
| ATOM | 7403 | N | GLN | A | 945 | 67.169 | 65.318 | −27.551 | 1.00 | 14.07 | A |
| ATOM | 7404 | CA | GLN | A | 945 | 66.180 | 64.328 | −27.168 | 1.00 | 14.57 | A |
| ATOM | 7405 | CB | GLN | A | 945 | 65.814 | 63.516 | −28.412 | 1.00 | 15.05 | A |
| ATOM | 7406 | CG | GLN | A | 945 | 64.863 | 62.360 | −28.204 | 1.00 | 17.81 | A |
| ATOM | 7407 | CD | GLN | A | 945 | 64.575 | 61.630 | −29.503 | 1.00 | 18.96 | A |
| ATOM | 7408 | OE1 | GLN | A | 945 | 63.803 | 62.104 | −30.341 | 1.00 | 21.23 | A |
| ATOM | 7409 | NE2 | GLN | A | 945 | 65.208 | 60.480 | −29.684 | 1.00 | 20.13 | A |
| ATOM | 7410 | C | GLN | A | 945 | 66.620 | 63.394 | −26.049 | 1.00 | 13.74 | A |
| ATOM | 7411 | O | GLN | A | 945 | 67.812 | 63.171 | −25.838 | 1.00 | 14.42 | A |
| ATOM | 7412 | N | PHE | A | 946 | 65.637 | 62.874 | −25.320 | 1.00 | 12.42 | A |
| ATOM | 7413 | CA | PHE | A | 946 | 65.871 | 61.913 | −24.249 | 1.00 | 11.92 | A |
| ATOM | 7414 | CB | PHE | A | 946 | 65.816 | 62.565 | −22.866 | 1.00 | 11.65 | A |
| ATOM | 7415 | CG | PHE | A | 946 | 65.710 | 61.567 | −21.742 | 1.00 | 11.33 | A |
| ATOM | 7416 | CD1 | PHE | A | 946 | 66.766 | 60.708 | −21.455 | 1.00 | 11.76 | A |
| ATOM | 7417 | CD2 | PHE | A | 946 | 64.532 | 61.447 | −21.013 | 1.00 | 11.56 | A |
| ATOM | 7418 | CE1 | PHE | A | 946 | 66.648 | 59.735 | −20.456 | 1.00 | 12.23 | A |
| ATOM | 7419 | CE2 | PHE | A | 946 | 64.402 | 60.481 | −20.016 | 1.00 | 11.37 | A |
| ATOM | 7420 | CZ | PHE | A | 946 | 65.463 | 59.623 | −19.738 | 1.00 | 11.69 | A |
| ATOM | 7421 | C | PHE | A | 946 | 64.765 | 60.872 | −24.338 | 1.00 | 11.81 | A |
| ATOM | 7422 | O | PHE | A | 946 | 63.599 | 61.216 | −24.522 | 1.00 | 11.13 | A |
| ATOM | 7423 | N | GLY | A | 947 | 65.134 | 59.600 | −24.222 | 1.00 | 11.32 | A |
| ATOM | 7424 | CA | GLY | A | 947 | 64.146 | 58.536 | −24.270 | 1.00 | 12.34 | A |
| ATOM | 7425 | C | GLY | A | 947 | 63.815 | 57.963 | −25.631 | 1.00 | 12.93 | A |
| ATOM | 7426 | O | GLY | A | 947 | 62.848 | 57.213 | −25.763 | 1.00 | 12.03 | A |
| ATOM | 7427 | N | GLY | A | 948 | 64.604 | 58.306 | −26.644 | 1.00 | 14.12 | A |
| ATOM | 7428 | CA | GLY | A | 948 | 64.346 | 57.786 | −27.973 | 1.00 | 15.63 | A |
| ATOM | 7429 | C | GLY | A | 948 | 64.401 | 56.271 | −28.021 | 1.00 | 17.10 | A |
| ATOM | 7430 | O | GLY | A | 948 | 63.854 | 55.652 | −28.934 | 1.00 | 18.33 | A |
| ATOM | 7431 | N | ASP | A | 949 | 65.055 | 55.671 | −27.033 | 1.00 | 18.00 | A |
| ATOM | 7432 | CA | ASP | A | 949 | 65.183 | 54.222 | −26.973 | 1.00 | 18.53 | A |
| ATOM | 7433 | CB | ASP | A | 949 | 66.614 | 53.841 | −26.570 | 1.00 | 20.30 | A |
| ATOM | 7434 | CG | ASP | A | 949 | 66.955 | 54.263 | −25.154 | 1.00 | 22.70 | A |
| ATOM | 7435 | OD1 | ASP | A | 949 | 66.613 | 55.400 | −24.764 | 1.00 | 24.14 | A |
| ATOM | 7436 | OD2 | ASP | A | 949 | 67.577 | 53.458 | −24.430 | 1.00 | 25.19 | A |
| ATOM | 7437 | C | ASP | A | 949 | 64.179 | 53.573 | −26.019 | 1.00 | 17.78 | A |
| ATOM | 7438 | O | ASP | A | 949 | 64.194 | 52.356 | −25.834 | 1.00 | 18.75 | A |
| ATOM | 7439 | N | HIS | A | 950 | 63.310 | 54.380 | −25.411 | 1.00 | 16.45 | A |
| ATOM | 7440 | CA | HIS | A | 950 | 62.297 | 53.841 | −24.503 | 1.00 | 14.81 | A |
| ATOM | 7441 | CB | HIS | A | 950 | 61.548 | 54.952 | −23.758 | 1.00 | 13.22 | A |
| ATOM | 7442 | CG | HIS | A | 950 | 62.378 | 55.720 | −22.778 | 1.00 | 12.93 | A |
| ATOM | 7443 | CD2 | HIS | A | 950 | 62.106 | 56.855 | −22.095 | 1.00 | 10.00 | A |
| ATOM | 7444 | ND1 | HIS | A | 950 | 63.630 | 55.316 | −22.365 | 1.00 | 13.22 | A |
| ATOM | 7445 | CE1 | HIS | A | 950 | 64.091 | 56.172 | −21.469 | 1.00 | 10.93 | A |
| ATOM | 7446 | NE2 | HIS | A | 950 | 63.185 | 57.115 | −21.287 | 1.00 | 13.26 | A |
| ATOM | 7447 | C | HIS | A | 950 | 61.263 | 53.076 | −25.326 | 1.00 | 14.43 | A |
| ATOM | 7448 | O | HIS | A | 950 | 60.886 | 53.508 | −26.414 | 1.00 | 15.25 | A |
| ATOM | 7449 | N | PRO | A | 951 | 60.788 | 51.931 | −24.818 | 1.00 | 15.24 | A |
| ATOM | 7450 | CD | PRO | A | 951 | 61.323 | 51.140 | −23.696 | 1.00 | 16.21 | A |
| ATOM | 7451 | CA | PRO | A | 951 | 59.788 | 51.164 | −25.566 | 1.00 | 14.93 | A |
| ATOM | 7452 | CB | PRO | A | 951 | 59.614 | 49.906 | −24.715 | 1.00 | 16.19 | A |
| ATOM | 7453 | CG | PRO | A | 951 | 60.975 | 49.727 | −24.112 | 1.00 | 16.73 | A |
| ATOM | 7454 | C | PRO | A | 951 | 58.484 | 51.952 | −25.692 | 1.00 | 14.35 | A |
| ATOM | 7455 | O | PRO | A | 951 | 58.084 | 52.657 | −24.762 | 1.00 | 14.20 | A |
| ATOM | 7456 | N | SER | A | 952 | 57.832 | 51.849 | −26.845 | 1.00 | 13.71 | A |
| ATOM | 7457 | CA | SER | A | 952 | 56.566 | 52.538 | −27.064 | 1.00 | 13.35 | A |
| ATOM | 7458 | CB | SER | A | 952 | 56.500 | 53.103 | −28.484 | 1.00 | 13.35 | A |
| ATOM | 7459 | OG | SER | A | 952 | 55.421 | 54.013 | −28.630 | 1.00 | 14.37 | A |
| ATOM | 7460 | C | SER | A | 952 | 55.489 | 51.482 | −26.851 | 1.00 | 13.53 | A |
| ATOM | 7461 | O | SER | A | 952 | 55.179 | 50.696 | −27.752 | 1.00 | 13.71 | A |
| ATOM | 7462 | N | ALA | A | 953 | 54.933 | 51.473 | −25.643 | 1.00 | 13.61 | A |
| ATOM | 7463 | CA | ALA | A | 953 | 53.924 | 50.504 | −25.236 | 1.00 | 13.14 | A |
| ATOM | 7464 | CB | ALA | A | 953 | 53.755 | 50.556 | −23.719 | 1.00 | 13.24 | A |
| ATOM | 7465 | C | ALA | A | 953 | 52.562 | 50.619 | −25.902 | 1.00 | 13.16 | A |
| ATOM | 7466 | O | ALA | A | 953 | 52.190 | 51.671 | −26.421 | 1.00 | 12.81 | A |
| ATOM | 7467 | N | ARG | A | 954 | 51.824 | 49.511 | −25.877 | 1.00 | 13.06 | A |
| ATOM | 7468 | CA | ARG | A | 954 | 50.486 | 49.452 | −26.446 | 1.00 | 14.74 | A |
| ATOM | 7469 | CB | ARG | A | 954 | 49.857 | 48.094 | −26.142 | 1.00 | 18.83 | A |
| ATOM | 7470 | CG | ARG | A | 954 | 48.477 | 47.891 | −26.737 | 1.00 | 25.14 | A |
| ATOM | 7471 | CD | ARG | A | 954 | 48.371 | 46.517 | −27.377 | 1.00 | 30.81 | A |
| ATOM | 7472 | NE | ARG | A | 954 | 46.988 | 46.066 | −27.486 | 1.00 | 35.73 | A |
| ATOM | 7473 | CZ | ARG | A | 954 | 46.241 | 45.713 | −26.447 | 1.00 | 38.06 | A |
| ATOM | 7474 | NH1 | ARG | A | 954 | 46.748 | 45.760 | −25.224 | 1.00 | 39.95 | A |
| ATOM | 7475 | NH2 | ARG | A | 954 | 44.990 | 45.309 | −26.628 | 1.00 | 39.73 | A |
| ATOM | 7476 | C | ARG | A | 954 | 49.658 | 50.583 | −25.837 | 1.00 | 12.99 | A |
| ATOM | 7477 | O | ARG | A | 954 | 49.792 | 50.893 | −24.651 | 1.00 | 12.00 | A |
| ATOM | 7478 | N | GLU | A | 955 | 48.792 | 51.178 | −26.652 | 1.00 | 12.43 | A |
| ATOM | 7479 | CA | GLU | A | 955 | 47.979 | 52.320 | −26.241 | 1.00 | 12.31 | A |
| ATOM | 7480 | CB | GLU | A | 955 | 47.073 | 52.751 | −27.400 | 1.00 | 13.73 | A |
| ATOM | 7481 | CG | GLU | A | 955 | 45.904 | 51.825 | −27.665 | 1.00 | 16.19 | A |
| ATOM | 7482 | CD | GLU | A | 955 | 45.042 | 52.303 | −28.814 | 1.00 | 17.59 | A |
| ATOM | 7483 | OE1 | GLU | A | 955 | 44.870 | 53.531 | −28.958 | 1.00 | 20.32 | A |
| ATOM | 7484 | OE2 | GLU | A | 955 | 44.528 | 51.454 | −29.567 | 1.00 | 19.35 | A |
| ATOM | 7485 | C | GLU | A | 955 | 47.140 | 52.203 | −24.970 | 1.00 | 11.08 | A |
| ATOM | 7486 | O | GLU | A | 955 | 46.859 | 53.216 | −24.326 | 1.00 | 10.87 | A |
| ATOM | 7487 | N | ASP | A | 956 | 46.734 | 50.992 | −24.602 | 1.00 | 10.57 | A |
| ATOM | 7488 | CA | ASP | A | 956 | 45.919 | 50.829 | −23.403 | 1.00 | 11.30 | A |
| ATOM | 7489 | CB | ASP | A | 956 | 44.937 | 49.656 | −23.567 | 1.00 | 12.11 | A |
| ATOM | 7490 | CG | ASP | A | 956 | 45.624 | 48.339 | −23.887 | 1.00 | 13.69 | A |
| ATOM | 7491 | OD1 | ASP | A | 956 | 46.860 | 48.317 | −24.078 | 1.00 | 14.76 | A |
| ATOM | 7492 | OD2 | ASP | A | 956 | 44.909 | 47.313 | −23.949 | 1.00 | 15.09 | A |
| ATOM | 7493 | C | ASP | A | 956 | 46.731 | 50.665 | −22.124 | 1.00 | 10.25 | A |
| ATOM | 7494 | O | ASP | A | 956 | 46.164 | 50.521 | −21.042 | 1.00 | 11.79 | A |
| ATOM | 7495 | N | LEU | A | 957 | 48.053 | 50.704 | −22.244 | 1.00 | 9.84 | A |
| ATOM | 7496 | CA | LEU | A | 957 | 48.922 | 50.558 | −21.080 | 1.00 | 9.73 | A |
| ATOM | 7497 | CB | LEU | A | 957 | 50.052 | 49.567 | −21.387 | 1.00 | 11.24 | A |
| ATOM | 7498 | CG | LEU | A | 957 | 50.977 | 49.172 | −20.230 | 1.00 | 12.99 | A |
| ATOM | 7499 | CD1 | LEU | A | 957 | 50.171 | 48.464 | −19.144 | 1.00 | 14.77 | A |
| ATOM | 7500 | CD2 | LEU | A | 957 | 52.086 | 48.257 | −20.742 | 1.00 | 13.87 | A |
| ATOM | 7501 | C | LEU | A | 957 | 49.524 | 51.894 | −20.668 | 1.00 | 9.37 | A |
| ATOM | 7502 | O | LEU | A | 957 | 49.927 | 52.694 | −21.512 | 1.00 | 10.03 | A |
| ATOM | 7503 | N | ASP | A | 958 | 49.582 | 52.139 | −19.366 | 1.00 | 8.45 | A |
| ATOM | 7504 | CA | ASP | A | 958 | 50.169 | 53.374 | −18.876 | 1.00 | 7.96 | A |
| ATOM | 7505 | CB | ASP | A | 958 | 49.071 | 54.379 | −18.495 | 1.00 | 8.11 | A |
| ATOM | 7506 | CG | ASP | A | 958 | 49.620 | 55.761 | −18.179 | 1.00 | 7.70 | A |
| ATOM | 7507 | OD1 | ASP | A | 958 | 50.731 | 56.094 | −18.644 | 1.00 | 8.38 | A |
| ATOM | 7508 | OD2 | ASP | A | 958 | 48.925 | 56.526 | −17.472 | 1.00 | 9.30 | A |
| ATOM | 7509 | C | ASP | A | 958 | 51.054 | 53.100 | −17.672 | 1.00 | 7.71 | A |
| ATOM | 7510 | O | ASP | A | 958 | 50.831 | 52.145 | −16.922 | 1.00 | 7.72 | A |
| ATOM | 7511 | N | VAL | A | 959 | 52.090 | 53.917 | −17.529 | 1.00 | 7.93 | A |
| ATOM | 7512 | CA | VAL | A | 959 | 52.982 | 53.840 | −16.381 | 1.00 | 7.45 | A |
| ATOM | 7513 | CB | VAL | A | 959 | 54.446 | 54.104 | −16.769 | 1.00 | 8.17 | A |
| ATOM | 7514 | CG1 | VAL | A | 959 | 55.310 | 54.221 | −15.508 | 1.00 | 8.69 | A |
| ATOM | 7515 | CG2 | VAL | A | 959 | 54.957 | 52.963 | −17.649 | 1.00 | 9.44 | A |
| ATOM | 7516 | C | VAL | A | 959 | 52.435 | 54.981 | −15.529 | 1.00 | 7.71 | A |
| ATOM | 7517 | O | VAL | A | 959 | 52.868 | 56.131 | −15.641 | 1.00 | 7.48 | A |
| ATOM | 7518 | N | SER | A | 960 | 51.437 | 54.651 | −14.717 | 1.00 | 8.00 | A |
| ATOM | 7519 | CA | SER | A | 960 | 50.759 | 55.617 | −13.860 | 1.00 | 8.19 | A |
| ATOM | 7520 | CB | SER | A | 960 | 49.654 | 54.919 | −13.065 | 1.00 | 8.71 | A |
| ATOM | 7521 | OG | SER | A | 960 | 48.832 | 54.137 | −13.917 | 1.00 | 9.90 | A |
| ATOM | 7522 | C | SER | A | 960 | 51.708 | 56.305 | −12.901 | 1.00 | 8.29 | A |
| ATOM | 7523 | O | SER | A | 960 | 51.571 | 57.500 | −12.626 | 1.00 | 7.81 | A |
| ATOM | 7524 | N | VAL | A | 961 | 52.664 | 55.537 | −12.391 | 1.00 | 8.17 | A |
| ATOM | 7525 | CA | VAL | A | 961 | 53.641 | 56.055 | −11.447 | 1.00 | 8.74 | A |
| ATOM | 7526 | CB | VAL | A | 961 | 53.272 | 55.682 | −9.984 | 1.00 | 9.01 | A |
| ATOM | 7527 | CG1 | VAL | A | 961 | 54.391 | 56.118 | −9.037 | 1.00 | 8.99 | A |
| ATOM | 7528 | CG2 | VAL | A | 961 | 51.954 | 56.332 | −9.576 | 1.00 | 9.73 | A |
| ATOM | 7529 | C | VAL | A | 961 | 55.039 | 55.502 | −11.685 | 1.00 | 8.56 | A |
| ATOM | 7530 | O | VAL | A | 961 | 55.211 | 54.314 | −11.966 | 1.00 | 8.35 | A |
| ATOM | 7531 | N | MSE | A | 962 | 56.025 | 56.388 | −11.601 | 1.00 | 7.97 | A |
| ATOM | 7532 | CA | MSE | A | 962 | 57.426 | 56.005 | −11.672 | 1.00 | 7.71 | A |
| ATOM | 7533 | CB | MSE | A | 962 | 58.105 | 56.387 | −12.982 | 1.00 | 9.07 | A |
| ATOM | 7534 | CG | MSE | A | 962 | 59.572 | 55.988 | −12.957 | 1.00 | 10.29 | A |
| ATOM | 7535 | SE | MSE | A | 962 | 60.468 | 56.332 | −14.600 | 1.00 | 16.74 | A |
| ATOM | 7536 | CE | MSE | A | 962 | 62.233 | 55.692 | −14.146 | 1.00 | 11.26 | A |
| ATOM | 7537 | C | MSE | A | 962 | 58.024 | 56.816 | −10.533 | 1.00 | 8.28 | A |
| ATOM | 7538 | O | MSE | A | 962 | 57.996 | 58.047 | −10.553 | 1.00 | 8.57 | A |
| ATOM | 7539 | N | ARG | A | 963 | 58.547 | 56.125 | −9.528 | 1.00 | 7.92 | A |
| ATOM | 7540 | CA | ARG | A | 963 | 59.101 | 56.792 | −8.357 | 1.00 | 8.31 | A |
| ATOM | 7541 | CB | ARG | A | 963 | 58.032 | 56.827 | −7.253 | 1.00 | 8.77 | A |
| ATOM | 7542 | CG | ARG | A | 963 | 58.504 | 57.343 | −5.892 | 1.00 | 9.81 | A |
| ATOM | 7543 | CD | ARG | A | 963 | 57.395 | 57.201 | −4.839 | 1.00 | 9.72 | A |
| ATOM | 7544 | NE | ARG | A | 963 | 56.184 | 57.886 | −5.277 | 1.00 | 7.97 | A |
| ATOM | 7545 | CZ | ARG | A | 963 | 54.952 | 57.396 | −5.169 | 1.00 | 7.38 | A |
| ATOM | 7546 | NH1 | ARG | A | 963 | 54.737 | 56.207 | −4.615 | 1.00 | 7.56 | A |
| ATOM | 7547 | NH2 | ARG | A | 963 | 53.932 | 58.084 | −5.670 | 1.00 | 7.55 | A |
| ATOM | 7548 | C | ARG | A | 963 | 60.339 | 56.101 | −7.815 | 1.00 | 7.62 | A |
| ATOM | 7549 | O | ARG | A | 963 | 60.291 | 54.920 | −7.493 | 1.00 | 7.67 | A |
| ATOM | 7550 | N | ARG | A | 964 | 61.449 | 56.828 | −7.716 | 1.00 | 8.28 | A |
| ATOM | 7551 | CA | ARG | A | 964 | 62.647 | 56.225 | −7.145 | 1.00 | 8.89 | A |
| ATOM | 7552 | CB | ARG | A | 964 | 63.884 | 57.089 | −7.404 | 1.00 | 8.93 | A |
| ATOM | 7553 | CG | ARG | A | 964 | 65.167 | 56.450 | −6.869 | 1.00 | 9.00 | A |
| ATOM | 7554 | CD | ARG | A | 964 | 66.407 | 57.176 | −7.367 | 1.00 | 10.27 | A |
| ATOM | 7555 | NE | ARG | A | 964 | 66.602 | 57.011 | −8.807 | 1.00 | 10.73 | A |
| ATOM | 7556 | CZ | ARG | A | 964 | 67.433 | 56.134 | −9.362 | 1.00 | 10.80 | A |
| ATOM | 7557 | NH1 | ARG | A | 964 | 68.166 | 55.324 | −8.605 | 1.00 | 12.81 | A |
| ATOM | 7558 | NH2 | ARG | A | 964 | 67.545 | 56.070 | −10.684 | 1.00 | 12.53 | A |
| ATOM | 7559 | C | ARG | A | 964 | 62.329 | 56.156 | −5.655 | 1.00 | 9.28 | A |
| ATOM | 7560 | O | ARG | A | 964 | 61.892 | 57.143 | −5.057 | 1.00 | 9.41 | A |
| ATOM | 7561 | N | LEU | A | 965 | 62.543 | 54.987 | −5.066 | 1.00 | 9.53 | A |
| ATOM | 7562 | CA | LEU | A | 965 | 62.217 | 54.754 | −3.663 | 1.00 | 9.91 | A |
| ATOM | 7563 | CB | LEU | A | 965 | 61.586 | 53.367 | −3.524 | 1.00 | 10.58 | A |
| ATOM | 7564 | CG | LEU | A | 965 | 60.391 | 53.090 | −4.442 | 1.00 | 10.29 | A |
| ATOM | 7565 | CD1 | LEU | A | 965 | 60.038 | 51.612 | −4.420 | 1.00 | 10.06 | A |
| ATOM | 7566 | CD2 | LEU | A | 965 | 59.205 | 53.929 | −4.000 | 1.00 | 11.47 | A |
| ATOM | 7567 | C | LEU | A | 965 | 63.387 | 54.869 | −2.695 | 1.00 | 10.70 | A |
| ATOM | 7568 | O | LEU | A | 965 | 63.196 | 54.816 | −1.478 | 1.00 | 11.63 | A |
| ATOM | 7569 | N | THR | A | 966 | 64.588 | 55.037 | −3.234 | 1.00 | 10.63 | A |
| ATOM | 7570 | CA | THR | A | 966 | 65.791 | 55.136 | −2.413 | 1.00 | 11.37 | A |
| ATOM | 7571 | CB | THR | A | 966 | 66.782 | 54.007 | −2.762 | 1.00 | 11.25 | A |
| ATOM | 7572 | OG1 | THR | A | 966 | 66.908 | 53.906 | −4.186 | 1.00 | 10.62 | A |
| ATOM | 7573 | CG2 | THR | A | 966 | 66.304 | 52.678 | −2.204 | 1.00 | 11.27 | A |
| ATOM | 7574 | C | THR | A | 966 | 66.527 | 56.454 | −2.599 | 1.00 | 11.74 | A |
| ATOM | 7575 | O | THR | A | 966 | 66.485 | 57.045 | −3.678 | 1.00 | 11.44 | A |
| ATOM | 7576 | N | LYS | A | 967 | 67.193 | 56.907 | −1.540 | 1.00 | 12.38 | A |
| ATOM | 7577 | CA | LYS | A | 967 | 67.986 | 58.129 | −1.591 | 1.00 | 13.90 | A |
| ATOM | 7578 | CB | LYS | A | 967 | 68.110 | 58.758 | −0.202 | 1.00 | 15.59 | A |
| ATOM | 7579 | CG | LYS | A | 967 | 66.814 | 59.377 | 0.312 | 1.00 | 18.36 | A |
| ATOM | 7580 | CD | LYS | A | 967 | 67.054 | 60.147 | 1.600 | 1.00 | 20.78 | A |
| ATOM | 7581 | CE | LYS | A | 967 | 65.797 | 60.856 | 2.074 | 1.00 | 22.33 | A |
| ATOM | 7582 | NZ | LYS | A | 967 | 66.063 | 61.668 | 3.296 | 1.00 | 24.16 | A |
| ATOM | 7583 | C | LYS | A | 967 | 69.361 | 57.728 | −2.129 | 1.00 | 14.35 | A |
| ATOM | 7584 | O | LYS | A | 967 | 69.669 | 56.541 | −2.218 | 1.00 | 13.76 | A |
| ATOM | 7585 | N | SER | A | 968 | 70.180 | 58.713 | −2.482 | 1.00 | 15.21 | A |
| ATOM | 7586 | CA | SER | A | 968 | 71.497 | 58.452 | −3.061 | 1.00 | 16.60 | A |
| ATOM | 7587 | CB | SER | A | 968 | 72.183 | 59.776 | −3.415 | 1.00 | 17.31 | A |
| ATOM | 7588 | OG | SER | A | 968 | 72.434 | 60.550 | −2.257 | 1.00 | 19.84 | A |
| ATOM | 7589 | C | SER | A | 968 | 72.464 | 57.592 | −2.250 | 1.00 | 16.96 | A |
| ATOM | 7590 | O | SER | A | 968 | 73.327 | 56.931 | −2.826 | 1.00 | 18.10 | A |
| ATOM | 7591 | N | SER | A | 969 | 72.320 | 57.593 | −0.929 | 1.00 | 17.39 | A |
| ATOM | 7592 | CA | SER | A | 969 | 73.214 | 56.819 | −0.065 | 1.00 | 18.70 | A |
| ATOM | 7593 | CB | SER | A | 969 | 73.108 | 57.320 | 1.376 | 1.00 | 19.87 | A |
| ATOM | 7594 | OG | SER | A | 969 | 71.782 | 57.194 | 1.862 | 1.00 | 23.45 | A |
| ATOM | 7595 | C | SER | A | 969 | 72.999 | 55.306 | −0.089 | 1.00 | 18.31 | A |
| ATOM | 7596 | O | SER | A | 969 | 73.839 | 54.552 | 0.408 | 1.00 | 18.36 | A |
| ATOM | 7597 | N | ALA | A | 970 | 71.887 | 54.856 | −0.662 | 1.00 | 17.75 | A |
| ATOM | 7598 | CA | ALA | A | 970 | 71.595 | 53.427 | −0.723 | 1.00 | 17.78 | A |
| ATOM | 7599 | CB | ALA | A | 970 | 70.111 | 53.207 | −0.997 | 1.00 | 17.81 | A |
| ATOM | 7600 | C | ALA | A | 970 | 72.427 | 52.697 | −1.772 | 1.00 | 18.18 | A |
| ATOM | 7601 | O | ALA | A | 970 | 72.379 | 53.027 | −2.959 | 1.00 | 18.06 | A |
| ATOM | 7602 | N | LYS | A | 971 | 73.185 | 51.696 | −1.328 | 1.00 | 18.93 | A |
| ATOM | 7603 | CA | LYS | A | 971 | 74.021 | 50.905 | −2.226 | 1.00 | 19.90 | A |
| ATOM | 7604 | CB | LYS | A | 971 | 74.726 | 49.793 | −1.447 | 1.00 | 21.65 | A |
| ATOM | 7605 | CG | LYS | A | 971 | 75.912 | 50.260 | −0.619 | 1.00 | 24.85 | A |
| ATOM | 7606 | CD | LYS | A | 971 | 76.437 | 49.145 | 0.278 | 1.00 | 25.97 | A |
| ATOM | 7607 | CE | LYS | A | 971 | 76.653 | 47.850 | −0.495 | 1.00 | 26.95 | A |
| ATOM | 7608 | NZ | LYS | A | 971 | 77.570 | 48.023 | −1.653 | 1.00 | 27.59 | A |
| ATOM | 7609 | C | LYS | A | 971 | 73.185 | 50.290 | −3.341 | 1.00 | 18.94 | A |
| ATOM | 7610 | O | LYS | A | 971 | 73.598 | 50.269 | −4.500 | 1.00 | 20.08 | A |
| ATOM | 7611 | N | THR | A | 972 | 72.013 | 49.778 | −2.982 | 1.00 | 18.05 | A |
| ATOM | 7612 | CA | THR | A | 972 | 71.116 | 49.182 | −3.961 | 1.00 | 16.90 | A |
| ATOM | 7613 | CB | THR | A | 972 | 70.563 | 47.820 | −3.483 | 1.00 | 17.94 | A |
| ATOM | 7614 | OG1 | THR | A | 972 | 71.649 | 46.913 | −3.252 | 1.00 | 19.39 | A |
| ATOM | 7615 | CG2 | THR | A | 972 | 69.641 | 47.222 | −4.538 | 1.00 | 18.08 | A |
| ATOM | 7616 | C | THR | A | 972 | 69.949 | 50.135 | −4.183 | 1.00 | 15.14 | A |
| ATOM | 7617 | O | THR | A | 972 | 69.116 | 50.327 | −3.298 | 1.00 | 14.90 | A |
| ATOM | 7618 | N | GLN | A | 973 | 69.903 | 50.746 | −5.361 | 1.00 | 13.21 | A |
| ATOM | 7619 | CA | GLN | A | 973 | 68.832 | 51.678 | −5.688 | 1.00 | 12.53 | A |
| ATOM | 7620 | CB | GLN | A | 973 | 69.267 | 52.585 | −6.841 | 1.00 | 11.85 | A |
| ATOM | 7621 | CG | GLN | A | 973 | 70.371 | 53.557 | −6.466 | 1.00 | 11.87 | A |
| ATOM | 7622 | CD | GLN | A | 973 | 69.924 | 54.569 | −5.432 | 1.00 | 12.68 | A |
| ATOM | 7623 | OE1 | GLN | A | 973 | 70.548 | 54.720 | −4.377 | 1.00 | 14.40 | A |
| ATOM | 7624 | NE2 | GLN | A | 973 | 68.840 | 55.275 | −5.731 | 1.00 | 10.57 | A |
| ATOM | 7625 | C | GLN | A | 973 | 67.565 | 50.923 | −6.062 | 1.00 | 11.92 | A |
| ATOM | 7626 | O | GLN | A | 973 | 67.623 | 49.851 | −6.656 | 1.00 | 12.62 | A |
| ATOM | 7627 | N | ARG | A | 974 | 66.415 | 51.485 | −5.706 | 1.00 | 11.58 | A |
| ATOM | 7628 | CA | ARG | A | 974 | 65.143 | 50.856 | −6.022 | 1.00 | 11.62 | A |
| ATOM | 7629 | CB | ARG | A | 974 | 64.485 | 50.318 | −4.750 | 1.00 | 12.80 | A |
| ATOM | 7630 | CG | ARG | A | 974 | 65.324 | 49.299 | −3.994 | 1.00 | 14.41 | A |
| ATOM | 7631 | CD | ARG | A | 974 | 64.713 | 48.990 | −2.633 | 1.00 | 15.71 | A |
| ATOM | 7632 | NE | ARG | A | 974 | 63.450 | 48.264 | −2.738 | 1.00 | 19.08 | A |
| ATOM | 7633 | CZ | ARG | A | 974 | 62.310 | 48.656 | −2.176 | 1.00 | 18.19 | A |
| ATOM | 7634 | NH1 | ARG | A | 974 | 62.263 | 49.776 | −1.466 | 1.00 | 19.24 | A |
| ATOM | 7635 | NH2 | ARG | A | 974 | 61.217 | 47.919 | −2.317 | 1.00 | 19.09 | A |
| ATOM | 7636 | C | ARG | A | 974 | 64.215 | 51.865 | −6.685 | 1.00 | 10.37 | A |
| ATOM | 7637 | O | ARG | A | 974 | 64.117 | 53.012 | −6.246 | 1.00 | 11.02 | A |
| ATOM | 7638 | N | VAL | A | 975 | 63.547 | 51.436 | −7.750 | 1.00 | 10.47 | A |
| ATOM | 7639 | CA | VAL | A | 975 | 62.612 | 52.300 | −8.458 | 1.00 | 10.13 | A |
| ATOM | 7640 | CB | VAL | A | 975 | 63.130 | 52.680 | −9.860 | 1.00 | 10.67 | A |
| ATOM | 7641 | CG1 | VAL | A | 975 | 62.104 | 53.560 | −10.579 | 1.00 | 10.06 | A |
| ATOM | 7642 | CG2 | VAL | A | 975 | 64.450 | 53.423 | −9.736 | 1.00 | 10.88 | A |
| ATOM | 7643 | C | VAL | A | 975 | 61.287 | 51.567 | −8.594 | 1.00 | 10.08 | A |
| ATOM | 7644 | O | VAL | A | 975 | 61.232 | 50.423 | −9.063 | 1.00 | 9.41 | A |
| ATOM | 7645 | N | GLY | A | 976 | 60.217 | 52.232 | −8.178 | 1.00 | 9.67 | A |
| ATOM | 7646 | CA | GLY | A | 976 | 58.904 | 51.622 | −8.247 | 1.00 | 9.24 | A |
| ATOM | 7647 | C | GLY | A | 976 | 58.067 | 52.121 | −9.405 | 1.00 | 8.66 | A |
| ATOM | 7648 | O | GLY | A | 976 | 58.142 | 53.290 | −9.793 | 1.00 | 8.68 | A |
| ATOM | 7649 | N | TYR | A | 977 | 57.268 | 51.218 | −9.958 | 1.00 | 8.45 | A |
| ATOM | 7650 | CA | TYR | A | 977 | 56.390 | 51.541 | −11.072 | 1.00 | 8.07 | A |
| ATOM | 7651 | CB | TYR | A | 977 | 56.874 | 50.892 | −12.374 | 1.00 | 8.64 | A |
| ATOM | 7652 | CG | TYR | A | 977 | 58.271 | 51.257 | −12.791 | 1.00 | 8.17 | A |
| ATOM | 7653 | CD1 | TYR | A | 977 | 59.371 | 50.548 | −12.312 | 1.00 | 8.99 | A |
| ATOM | 7654 | CE1 | TYR | A | 977 | 60.664 | 50.885 | −12.706 | 1.00 | 9.30 | A |
| ATOM | 7655 | CD2 | TYR | A | 977 | 58.496 | 52.314 | −13.673 | 1.00 | 8.85 | A |
| ATOM | 7656 | CE2 | TYR | A | 977 | 59.773 | 52.662 | −14.068 | 1.00 | 10.31 | A |
| ATOM | 7657 | CZ | TYR | A | 977 | 60.855 | 51.947 | −13.586 | 1.00 | 9.76 | A |
| ATOM | 7658 | OH | TYR | A | 977 | 62.125 | 52.302 | −13.980 | 1.00 | 11.36 | A |
| ATOM | 7659 | C | TYR | A | 977 | 54.993 | 51.012 | −10.822 | 1.00 | 7.74 | A |
| ATOM | 7660 | O | TYR | A | 977 | 54.828 | 49.906 | −10.307 | 1.00 | 8.14 | A |
| ATOM | 7661 | N | VAL | A | 978 | 53.987 | 51.807 | −11.174 | 1.00 | 7.18 | A |
| ATOM | 7662 | CA | VAL | A | 978 | 52.613 | 51.346 | −11.084 | 1.00 | 7.63 | A |
| ATOM | 7663 | CB | VAL | A | 978 | 51.693 | 52.298 | −10.300 | 1.00 | 7.48 | A |
| ATOM | 7664 | CG1 | VAL | A | 978 | 50.250 | 51.818 | −10.418 | 1.00 | 8.22 | A |
| ATOM | 7665 | CG2 | VAL | A | 978 | 52.098 | 52.321 | −8.837 | 1.00 | 8.52 | A |
| ATOM | 7666 | C | VAL | A | 978 | 52.168 | 51.312 | −12.537 | 1.00 | 7.85 | A |
| ATOM | 7667 | O | VAL | A | 978 | 52.203 | 52.333 | −13.225 | 1.00 | 8.22 | A |
| ATOM | 7668 | N | LEU | A | 979 | 51.794 | 50.126 | −13.000 | 1.00 | 8.70 | A |
| ATOM | 7669 | CA | LEU | A | 979 | 51.350 | 49.919 | −14.369 | 1.00 | 10.20 | A |
| ATOM | 7670 | CB | LEU | A | 979 | 52.084 | 48.724 | −14.982 | 1.00 | 12.54 | A |
| ATOM | 7671 | CG | LEU | A | 979 | 53.417 | 48.976 | −15.684 | 1.00 | 16.22 | A |
| ATOM | 7672 | CD1 | LEU | A | 979 | 53.139 | 49.476 | −17.089 | 1.00 | 16.69 | A |
| ATOM | 7673 | CD2 | LEU | A | 979 | 54.262 | 49.969 | −14.897 | 1.00 | 16.92 | A |
| ATOM | 7674 | C | LEU | A | 979 | 49.856 | 49.663 | −14.417 | 1.00 | 9.64 | A |
| ATOM | 7675 | O | LEU | A | 979 | 49.343 | 48.787 | −13.724 | 1.00 | 10.75 | A |
| ATOM | 7676 | N | HIS | A | 980 | 49.152 | 50.435 | −15.233 | 1.00 | 9.35 | A |
| ATOM | 7677 | CA | HIS | A | 980 | 47.724 | 50.240 | −15.370 | 1.00 | 9.64 | A |
| ATOM | 7678 | CB | HIS | A | 980 | 46.926 | 51.443 | −14.863 | 1.00 | 9.78 | A |
| ATOM | 7679 | CG | HIS | A | 980 | 45.447 | 51.254 | −14.994 | 1.00 | 9.39 | A |
| ATOM | 7680 | CD2 | HIS | A | 980 | 44.602 | 50.431 | −14.332 | 1.00 | 7.39 | A |
| ATOM | 7681 | ND1 | HIS | A | 980 | 44.693 | 51.871 | −15.969 | 1.00 | 10.35 | A |
| ATOM | 7682 | CE1 | HIS | A | 980 | 43.448 | 51.432 | −15.903 | 1.00 | 7.04 | A |
| ATOM | 7683 | NE2 | HIS | A | 980 | 43.367 | 50.555 | −14.918 | 1.00 | 10.79 | A |
| ATOM | 7684 | C | HIS | A | 980 | 47.348 | 50.005 | −16.817 | 1.00 | 10.11 | A |
| ATOM | 7685 | O | HIS | A | 980 | 47.788 | 50.728 | −17.705 | 1.00 | 11.06 | A |
| ATOM | 7686 | N | ARG | A | 981 | 46.537 | 48.984 | −17.052 | 1.00 | 9.65 | A |
| ATOM | 7687 | CA | ARG | A | 981 | 46.076 | 48.711 | −18.397 | 1.00 | 10.37 | A |
| ATOM | 7688 | CB | ARG | A | 981 | 46.436 | 47.295 | −18.832 | 1.00 | 12.59 | A |
| ATOM | 7689 | CG | ARG | A | 981 | 46.052 | 46.995 | −20.272 | 1.00 | 16.41 | A |
| ATOM | 7690 | CD | ARG | A | 981 | 46.568 | 45.634 | −20.691 | 1.00 | 19.84 | A |
| ATOM | 7691 | NE | ARG | A | 981 | 46.118 | 45.254 | −22.024 | 1.00 | 23.55 | A |
| ATOM | 7692 | CZ | ARG | A | 981 | 46.338 | 44.058 | −22.564 | 1.00 | 25.07 | A |
| ATOM | 7693 | NH1 | ARG | A | 981 | 47.001 | 43.135 | −21.880 | 1.00 | 25.67 | A |
| ATOM | 7694 | NH2 | ARG | A | 981 | 45.890 | 43.780 | −23.782 | 1.00 | 26.12 | A |
| ATOM | 7695 | C | ARG | A | 981 | 44.572 | 48.872 | −18.360 | 1.00 | 9.55 | A |
| ATOM | 7696 | O | ARG | A | 981 | 43.892 | 48.218 | −17.576 | 1.00 | 9.46 | A |
| ATOM | 7697 | N | THR | A | 982 | 44.056 | 49.770 | −19.185 | 1.00 | 9.57 | A |
| ATOM | 7698 | CA | THR | A | 982 | 42.621 | 49.990 | −19.245 | 1.00 | 9.61 | A |
| ATOM | 7699 | CB | THR | A | 982 | 42.322 | 51.447 | −19.679 | 1.00 | 9.52 | A |
| ATOM | 7700 | OG1 | THR | A | 982 | 40.921 | 51.716 | −19.547 | 1.00 | 9.48 | A |
| ATOM | 7701 | CG2 | THR | A | 982 | 42.772 | 51.685 | −21.120 | 1.00 | 10.43 | A |
| ATOM | 7702 | C | THR | A | 982 | 42.095 | 48.993 | −20.280 | 1.00 | 10.60 | A |
| ATOM | 7703 | O | THR | A | 982 | 42.833 | 48.111 | −20.716 | 1.00 | 11.44 | A |
| ATOM | 7704 | N | ASN | A | 983 | 40.824 | 49.100 | −20.642 | 1.00 | 9.38 | A |
| ATOM | 7705 | CA | ASN | A | 983 | 40.261 | 48.211 | −21.652 | 1.00 | 10.07 | A |
| ATOM | 7706 | CB | ASN | A | 983 | 39.252 | 47.227 | −21.057 | 1.00 | 9.79 | A |
| ATOM | 7707 | CG | ASN | A | 983 | 38.746 | 46.233 | −22.088 | 1.00 | 9.90 | A |
| ATOM | 7708 | OD1 | ASN | A | 983 | 39.470 | 45.322 | −22.493 | 1.00 | 11.26 | A |
| ATOM | 7709 | ND2 | ASN | A | 983 | 37.510 | 46.418 | −22.535 | 1.00 | 11.73 | A |
| ATOM | 7710 | C | ASN | A | 983 | 39.560 | 49.077 | −22.682 | 1.00 | 10.88 | A |
| ATOM | 7711 | O | ASN | A | 983 | 38.624 | 49.811 | −22.359 | 1.00 | 10.83 | A |
| ATOM | 7712 | N | LEU | A | 984 | 40.029 | 48.994 | −23.920 | 1.00 | 11.31 | A |
| ATOM | 7713 | CA | LEU | A | 984 | 39.461 | 49.778 | −25.003 | 1.00 | 13.01 | A |
| ATOM | 7714 | CB | LEU | A | 984 | 40.574 | 50.464 | −25.790 | 1.00 | 13.38 | A |
| ATOM | 7715 | CG | LEU | A | 984 | 41.532 | 51.306 | −24.945 | 1.00 | 13.55 | A |
| ATOM | 7716 | CD1 | LEU | A | 984 | 42.655 | 51.828 | −25.825 | 1.00 | 14.92 | A |
| ATOM | 7717 | CD2 | LEU | A | 984 | 40.774 | 52.448 | −24.282 | 1.00 | 13.40 | A |
| ATOM | 7718 | C | LEU | A | 984 | 38.674 | 48.868 | −25.919 | 1.00 | 14.57 | A |
| ATOM | 7719 | O | LEU | A | 984 | 39.067 | 47.731 | −26.167 | 1.00 | 14.53 | A |
| ATOM | 7720 | N | MSE | A | 985 | 37.557 | 49.371 | −26.422 | 1.00 | 16.64 | A |
| ATOM | 7721 | CA | MSE | A | 985 | 36.724 | 48.579 | −27.307 | 1.00 | 20.14 | A |
| ATOM | 7722 | CB | MSE | A | 985 | 35.417 | 49.300 | −27.593 | 1.00 | 22.91 | A |
| ATOM | 7723 | CG | MSE | A | 985 | 34.540 | 49.482 | −26.392 | 1.00 | 26.23 | A |
| ATOM | 7724 | SE | MSE | A | 985 | 32.730 | 49.389 | −26.975 | 1.00 | 32.71 | A |
| ATOM | 7725 | CE | MSE | A | 985 | 32.637 | 51.060 | −27.940 | 1.00 | 29.41 | A |
| ATOM | 7726 | C | MSE | A | 985 | 37.403 | 48.274 | −28.625 | 1.00 | 21.11 | A |
| ATOM | 7727 | O | MSE | A | 985 | 38.127 | 49.101 | −29.178 | 1.00 | 21.04 | A |
| ATOM | 7728 | N | GLN | A | 986 | 37.169 | 47.070 | −29.124 | 1.00 | 22.25 | A |
| ATOM | 7729 | CA | GLN | A | 986 | 37.721 | 46.669 | −30.403 | 1.00 | 23.97 | A |
| ATOM | 7730 | CB | GLN | A | 986 | 38.052 | 45.174 | −30.389 | 1.00 | 26.84 | A |
| ATOM | 7731 | CG | GLN | A | 986 | 36.990 | 44.298 | −29.749 | 1.00 | 30.56 | A |
| ATOM | 7732 | CD | GLN | A | 986 | 37.548 | 42.971 | −29.263 | 1.00 | 32.21 | A |
| ATOM | 7733 | OE1 | GLN | A | 986 | 36.814 | 42.126 | −28.750 | 1.00 | 33.51 | A |
| ATOM | 7734 | NE2 | GLN | A | 986 | 38.856 | 42.785 | −29.416 | 1.00 | 33.24 | A |
| ATOM | 7735 | C | GLN | A | 986 | 36.626 | 46.995 | −31.407 | 1.00 | 23.34 | A |
| ATOM | 7736 | O | GLN | A | 986 | 35.513 | 46.474 | −31.314 | 1.00 | 23.11 | A |
| ATOM | 7737 | N | CYS | A | 987 | 36.929 | 47.885 | −32.346 | 1.00 | 22.85 | A |
| ATOM | 7738 | CA | CYS | A | 987 | 35.944 | 48.289 | −33.338 | 1.00 | 23.08 | A |
| ATOM | 7739 | C | CYS | A | 987 | 36.405 | 48.051 | −34.771 | 1.00 | 24.34 | A |
| ATOM | 7740 | O | CYS | A | 987 | 35.897 | 48.679 | −35.701 | 1.00 | 24.34 | A |
| ATOM | 7741 | CB | CYS | A | 987 | 35.592 | 49.767 | −33.160 | 1.00 | 22.10 | A |
| ATOM | 7742 | SG | CYS | A | 987 | 35.108 | 50.279 | −31.476 | 1.00 | 21.61 | A |
| ATOM | 7743 | N | GLY | A | 988 | 37.371 | 47.156 | −34.949 | 1.00 | 25.32 | A |
| ATOM | 7744 | CA | GLY | A | 988 | 37.845 | 46.849 | −36.287 | 1.00 | 28.05 | A |
| ATOM | 7745 | C | GLY | A | 988 | 39.102 | 47.560 | −36.744 | 1.00 | 29.88 | A |
| ATOM | 7746 | O | GLY | A | 988 | 39.416 | 47.558 | −37.937 | 1.00 | 29.77 | A |
| ATOM | 7747 | N | THR | A | 989 | 39.826 | 48.170 | −35.813 | 1.00 | 31.64 | A |
| ATOM | 7748 | CA | THR | A | 989 | 41.057 | 48.868 | −36.159 | 1.00 | 33.74 | A |
| ATOM | 7749 | CB | THR | A | 989 | 41.213 | 50.170 | −35.354 | 1.00 | 33.53 | A |
| ATOM | 7750 | OG1 | THR | A | 989 | 40.077 | 51.012 | −35.584 | 1.00 | 34.03 | A |
| ATOM | 7751 | CG2 | THR | A | 989 | 42.472 | 50.909 | −35.781 | 1.00 | 33.57 | A |
| ATOM | 7752 | C | THR | A | 989 | 42.253 | 47.967 | −35.875 | 1.00 | 35.49 | A |
| ATOM | 7753 | O | THR | A | 989 | 42.463 | 47.537 | −34.741 | 1.00 | 35.26 | A |
| ATOM | 7754 | N | PRO | A | 990 | 43.052 | 47.666 | −36.912 | 1.00 | 37.38 | A |
| ATOM | 7755 | CD | PRO | A | 990 | 42.900 | 48.144 | −38.298 | 1.00 | 37.60 | A |
| ATOM | 7756 | CA | PRO | A | 990 | 44.236 | 46.810 | −36.788 | 1.00 | 39.24 | A |
| ATOM | 7757 | CB | PRO | A | 990 | 44.979 | 47.076 | −38.090 | 1.00 | 38.87 | A |
| ATOM | 7758 | CG | PRO | A | 990 | 43.853 | 47.242 | −39.061 | 1.00 | 38.12 | A |
| ATOM | 7759 | C | PRO | A | 990 | 45.087 | 47.105 | −35.553 | 1.00 | 41.21 | A |
| ATOM | 7760 | O | PRO | A | 990 | 45.363 | 46.205 | −34.759 | 1.00 | 41.51 | A |
| ATOM | 7761 | N | GLU | A | 991 | 45.499 | 48.360 | −35.395 | 1.00 | 43.24 | A |
| ATOM | 7762 | CA | GLU | A | 991 | 46.316 | 48.756 | −34.250 | 1.00 | 45.36 | A |
| ATOM | 7763 | CB | GLU | A | 991 | 45.471 | 48.710 | −32.975 | 1.00 | 46.82 | A |
| ATOM | 7764 | CG | GLU | A | 991 | 44.233 | 49.595 | −33.029 | 1.00 | 48.73 | A |
| ATOM | 7765 | CD | GLU | A | 991 | 43.276 | 49.336 | −31.881 | 1.00 | 49.86 | A |
| ATOM | 7766 | OE1 | GLU | A | 991 | 43.701 | 49.450 | −30.713 | 1.00 | 50.20 | A |
| ATOM | 7767 | OE2 | GLU | A | 991 | 42.097 | 49.018 | −32.149 | 1.00 | 50.38 | A |
| ATOM | 7768 | C | GLU | A | 991 | 47.513 | 47.816 | −34.120 | 1.00 | 45.81 | A |
| ATOM | 7769 | O | GLU | A | 991 | 47.428 | 46.774 | −33.470 | 1.00 | 46.00 | A |
| ATOM | 7770 | N | GLU | A | 992 | 48.630 | 48.193 | −34.733 | 1.00 | 46.27 | A |
| ATOM | 7771 | CA | GLU | A | 992 | 49.825 | 47.359 | −34.705 | 1.00 | 46.79 | A |
| ATOM | 7772 | CB | GLU | A | 992 | 50.172 | 46.919 | −36.130 | 1.00 | 47.94 | A |
| ATOM | 7773 | CG | GLU | A | 992 | 49.080 | 46.124 | −36.825 | 1.00 | 49.54 | A |
| ATOM | 7774 | CD | GLU | A | 992 | 49.384 | 45.880 | −38.291 | 1.00 | 50.48 | A |
| ATOM | 7775 | OE1 | GLU | A | 992 | 50.469 | 45.341 | −38.596 | 1.00 | 50.94 | A |
| ATOM | 7776 | OE2 | GLU | A | 992 | 48.534 | 46.226 | −39.140 | 1.00 | 51.09 | A |
| ATOM | 7777 | C | GLU | A | 992 | 51.052 | 48.015 | −34.075 | 1.00 | 46.42 | A |
| ATOM | 7778 | O | GLU | A | 992 | 50.947 | 48.832 | −33.161 | 1.00 | 46.39 | A |
| ATOM | 7779 | N | HIS | A | 993 | 52.214 | 47.625 | −34.593 | 1.00 | 45.82 | A |
| ATOM | 7780 | CA | HIS | A | 993 | 53.526 | 48.105 | −34.167 | 1.00 | 44.90 | A |
| ATOM | 7781 | CB | HIS | A | 993 | 54.113 | 49.027 | −35.249 | 1.00 | 46.12 | A |
| ATOM | 7782 | CG | HIS | A | 993 | 53.206 | 50.144 | −35.668 | 1.00 | 47.42 | A |
| ATOM | 7783 | CD2 | HIS | A | 993 | 52.148 | 50.721 | −35.049 | 1.00 | 47.85 | A |
| ATOM | 7784 | ND1 | HIS | A | 993 | 53.364 | 50.818 | −36.860 | 1.00 | 47.77 | A |
| ATOM | 7785 | CE1 | HIS | A | 993 | 52.442 | 51.758 | −36.959 | 1.00 | 48.01 | A |
| ATOM | 7786 | NE2 | HIS | A | 993 | 51.691 | 51.721 | −35.873 | 1.00 | 48.09 | A |
| ATOM | 7787 | C | HIS | A | 993 | 53.669 | 48.760 | −32.794 | 1.00 | 43.41 | A |
| ATOM | 7788 | O | HIS | A | 993 | 53.618 | 49.983 | −32.662 | 1.00 | 43.54 | A |
| ATOM | 7789 | N | THR | A | 994 | 53.858 | 47.923 | −31.776 | 1.00 | 41.20 | A |
| ATOM | 7790 | CA | THR | A | 994 | 54.058 | 48.377 | −30.401 | 1.00 | 38.33 | A |
| ATOM | 7791 | CB | THR | A | 994 | 52.741 | 48.405 | −29.592 | 1.00 | 38.20 | A |
| ATOM | 7792 | OG1 | THR | A | 994 | 52.211 | 47.079 | −29.480 | 1.00 | 37.64 | A |
| ATOM | 7793 | CG2 | THR | A | 994 | 51.721 | 49.307 | −30.267 | 1.00 | 37.89 | A |
| ATOM | 7794 | C | THR | A | 994 | 55.028 | 47.405 | −29.734 | 1.00 | 36.67 | A |
| ATOM | 7795 | O | THR | A | 994 | 54.980 | 46.200 | −29.982 | 1.00 | 36.66 | A |
| ATOM | 7796 | N | GLN | A | 995 | 55.908 | 47.931 | −28.889 | 1.00 | 34.18 | A |
| ATOM | 7797 | CA | GLN | A | 995 | 56.899 | 47.105 | −28.210 | 1.00 | 31.74 | A |
| ATOM | 7798 | CB | GLN | A | 995 | 58.227 | 47.851 | −28.120 | 1.00 | 32.78 | A |
| ATOM | 7799 | CG | GLN | A | 995 | 58.693 | 48.461 | −29.423 | 1.00 | 34.00 | A |
| ATOM | 7800 | CD | GLN | A | 995 | 59.846 | 49.417 | −29.219 | 1.00 | 35.18 | A |
| ATOM | 7801 | OE1 | GLN | A | 995 | 60.953 | 49.009 | −28.862 | 1.00 | 35.89 | A |
| ATOM | 7802 | NE2 | GLN | A | 995 | 59.589 | 50.703 | −29.428 | 1.00 | 34.40 | A |
| ATOM | 7803 | C | GLN | A | 995 | 56.467 | 46.719 | −26.804 | 1.00 | 29.41 | A |
| ATOM | 7804 | O | GLN | A | 995 | 55.719 | 47.446 | −26.150 | 1.00 | 28.31 | A |
| ATOM | 7805 | N | LYS | A | 996 | 56.952 | 45.573 | −26.341 | 1.00 | 27.07 | A |
| ATOM | 7806 | CA | LYS | A | 996 | 56.639 | 45.110 | −25.000 | 1.00 | 25.50 | A |
| ATOM | 7807 | CB | LYS | A | 996 | 57.143 | 43.678 | −24.793 | 1.00 | 27.59 | A |
| ATOM | 7808 | CG | LYS | A | 996 | 56.424 | 42.636 | −25.635 | 1.00 | 30.19 | A |
| ATOM | 7809 | CD | LYS | A | 996 | 54.951 | 42.545 | −25.263 | 1.00 | 31.86 | A |
| ATOM | 7810 | CE | LYS | A | 996 | 54.246 | 41.461 | −26.063 | 1.00 | 32.41 | A |
| ATOM | 7811 | NZ | LYS | A | 996 | 54.837 | 40.117 | −25.811 | 1.00 | 33.07 | A |
| ATOM | 7812 | C | LYS | A | 996 | 57.333 | 46.045 | −24.024 | 1.00 | 23.02 | A |
| ATOM | 7813 | O | LYS | A | 996 | 58.498 | 46.399 | −24.210 | 1.00 | 21.73 | A |
| ATOM | 7814 | N | LEU | A | 997 | 56.613 | 46.456 | −22.989 | 1.00 | 20.46 | A |
| ATOM | 7815 | CA | LEU | A | 997 | 57.183 | 47.350 | −21.998 | 1.00 | 18.95 | A |
| ATOM | 7816 | CB | LEU | A | 997 | 56.180 | 48.443 | −21.622 | 1.00 | 18.97 | A |
| ATOM | 7817 | CG | LEU | A | 997 | 56.621 | 49.367 | −20.483 | 1.00 | 19.50 | A |
| ATOM | 7818 | CD1 | LEU | A | 997 | 57.859 | 50.141 | −20.903 | 1.00 | 19.83 | A |
| ATOM | 7819 | CD2 | LEU | A | 997 | 55.493 | 50.316 | −20.125 | 1.00 | 19.35 | A |
| ATOM | 7820 | C | LEU | A | 997 | 57.582 | 46.588 | −20.748 | 1.00 | 17.48 | A |
| ATOM | 7821 | O | LEU | A | 997 | 56.732 | 46.021 | −20.065 | 1.00 | 17.65 | A |
| ATOM | 7822 | N | ASP | A | 998 | 58.880 | 46.566 | −20.467 | 1.00 | 16.19 | A |
| ATOM | 7823 | CA | ASP | A | 998 | 59.399 | 45.910 | −19.273 | 1.00 | 15.60 | A |
| ATOM | 7824 | CB | ASP | A | 998 | 60.500 | 44.912 | −19.633 | 1.00 | 16.76 | A |
| ATOM | 7825 | CG | ASP | A | 998 | 61.145 | 44.295 | −18.408 | 1.00 | 17.89 | A |
| ATOM | 7826 | OD1 | ASP | A | 998 | 62.170 | 43.600 | −18.564 | 1.00 | 18.83 | A |
| ATOM | 7827 | OD2 | ASP | A | 998 | 60.626 | 44.503 | −17.289 | 1.00 | 17.40 | A |
| ATOM | 7828 | C | ASP | A | 998 | 59.976 | 47.028 | −18.414 | 1.00 | 14.75 | A |
| ATOM | 7829 | O | ASP | A | 998 | 61.117 | 47.447 | −18.609 | 1.00 | 14.20 | A |
| ATOM | 7830 | N | VAL | A | 999 | 59.185 | 47.521 | −17.469 | 1.00 | 14.42 | A |
| ATOM | 7831 | CA | VAL | A | 999 | 59.642 | 48.617 | −16.628 | 1.00 | 14.11 | A |
| ATOM | 7832 | CB | VAL | A | 999 | 58.532 | 49.089 | −15.655 | 1.00 | 13.61 | A |
| ATOM | 7833 | CG1 | VAL | A | 999 | 57.346 | 49.614 | −16.447 | 1.00 | 14.23 | A |
| ATOM | 7834 | CG2 | VAL | A | 999 | 58.109 | 47.955 | −14.738 | 1.00 | 14.31 | A |
| ATOM | 7835 | C | VAL | A | 999 | 60.897 | 48.299 | −15.827 | 1.00 | 14.57 | A |
| ATOM | 7836 | O | VAL | A | 999 | 61.669 | 49.195 | −15.494 | 1.00 | 14.03 | A |
| ATOM | 7837 | N | CYS | A | 1000 | 61.116 | 47.027 | −15.527 | 1.00 | 15.40 | A |
| ATOM | 7838 | CA | CYS | A | 1000 | 62.288 | 46.673 | −14.750 | 1.00 | 16.88 | A |
| ATOM | 7839 | C | CYS | A | 1000 | 63.609 | 46.871 | −15.487 | 1.00 | 16.40 | A |
| ATOM | 7840 | O | CYS | A | 1000 | 64.666 | 46.894 | −14.864 | 1.00 | 16.72 | A |
| ATOM | 7841 | CB | CYS | A | 1000 | 62.159 | 45.245 | −14.220 | 1.00 | 17.93 | A |
| ATOM | 7842 | SG | CYS | A | 1000 | 61.365 | 45.181 | −12.575 | 1.00 | 21.77 | A |
| ATOM | 7843 | N | HIS | A | 1001 | 63.555 | 47.033 | −16.806 | 1.00 | 16.66 | A |
| ATOM | 7844 | CA | HIS | A | 1001 | 64.779 | 47.258 | −17.569 | 1.00 | 17.02 | A |
| ATOM | 7845 | CB | HIS | A | 1001 | 64.903 | 46.251 | −18.720 | 1.00 | 17.37 | A |
| ATOM | 7846 | CG | HIS | A | 1001 | 65.459 | 44.925 | −18.303 | 1.00 | 18.58 | A |
| ATOM | 7847 | CD2 | HIS | A | 1001 | 66.716 | 44.427 | −18.368 | 1.00 | 18.89 | A |
| ATOM | 7848 | ND1 | HIS | A | 1001 | 64.693 | 43.951 | −17.698 | 1.00 | 18.64 | A |
| ATOM | 7849 | CE1 | HIS | A | 1001 | 65.455 | 42.911 | −17.408 | 1.00 | 19.12 | A |
| ATOM | 7850 | NE2 | HIS | A | 1001 | 66.687 | 43.174 | −17.804 | 1.00 | 19.79 | A |
| ATOM | 7851 | C | HIS | A | 1001 | 64.896 | 48.684 | −18.109 | 1.00 | 17.08 | A |
| ATOM | 7852 | O | HIS | A | 1001 | 65.742 | 48.962 | −18.960 | 1.00 | 17.52 | A |
| ATOM | 7853 | N | LEU | A | 1002 | 64.053 | 49.588 | −17.615 | 1.00 | 16.75 | A |
| ATOM | 7854 | CA | LEU | A | 1002 | 64.101 | 50.981 | −18.057 | 1.00 | 16.76 | A |
| ATOM | 7855 | CB | LEU | A | 1002 | 62.885 | 51.754 | −17.545 | 1.00 | 16.55 | A |
| ATOM | 7856 | CG | LEU | A | 1002 | 61.597 | 51.523 | −18.337 | 1.00 | 15.47 | A |
| ATOM | 7857 | CD1 | LEU | A | 1002 | 60.434 | 52.218 | −17.648 | 1.00 | 15.32 | A |
| ATOM | 7858 | CD2 | LEU | A | 1002 | 61.775 | 52.056 | −19.754 | 1.00 | 16.05 | A |
| ATOM | 7859 | C | LEU | A | 1002 | 65.380 | 51.646 | −17.566 | 1.00 | 17.51 | A |
| ATOM | 7860 | O | LEU | A | 1002 | 65.901 | 52.561 | −18.205 | 1.00 | 18.66 | A |
| ATOM | 7861 | N | LEU | A | 1003 | 65.869 | 51.190 | −16.418 | 1.00 | 17.61 | A |
| ATOM | 7862 | CA | LEU | A | 1003 | 67.108 | 51.704 | −15.847 | 1.00 | 18.02 | A |
| ATOM | 7863 | CB | LEU | A | 1003 | 66.906 | 52.109 | −14.387 | 1.00 | 18.59 | A |
| ATOM | 7864 | CG | LEU | A | 1003 | 66.180 | 53.442 | −14.184 | 1.00 | 20.43 | A |
| ATOM | 7865 | CD1 | LEU | A | 1003 | 65.881 | 53.644 | −12.717 | 1.00 | 20.90 | A |
| ATOM | 7866 | CD2 | LEU | A | 1003 | 67.041 | 54.581 | −14.716 | 1.00 | 20.41 | A |
| ATOM | 7867 | C | LEU | A | 1003 | 68.135 | 50.583 | −15.957 | 1.00 | 18.42 | A |
| ATOM | 7868 | O | LEU | A | 1003 | 67.802 | 49.409 | −15.805 | 1.00 | 17.92 | A |
| ATOM | 7869 | N | PRO | A | 1004 | 69.399 | 50.930 | −16.229 | 1.00 | 18.76 | A |
| ATOM | 7870 | CD | PRO | A | 1004 | 69.943 | 52.285 | −16.433 | 1.00 | 19.13 | A |
| ATOM | 7871 | CA | PRO | A | 1004 | 70.453 | 49.923 | −16.364 | 1.00 | 18.84 | A |
| ATOM | 7872 | CB | PRO | A | 1004 | 71.580 | 50.708 | −17.022 | 1.00 | 19.50 | A |
| ATOM | 7873 | CG | PRO | A | 1004 | 71.443 | 52.046 | −16.377 | 1.00 | 19.67 | A |
| ATOM | 7874 | C | PRO | A | 1004 | 70.903 | 49.252 | −15.071 | 1.00 | 19.22 | A |
| ATOM | 7875 | O | PRO | A | 1004 | 70.568 | 49.692 | −13.968 | 1.00 | 18.35 | A |
| ATOM | 7876 | N | ASN | A | 1005 | 71.658 | 48.168 | −15.233 | 1.00 | 19.25 | A |
| ATOM | 7877 | CA | ASN | A | 1005 | 72.210 | 47.420 | −14.112 | 1.00 | 19.59 | A |
| ATOM | 7878 | CB | ASN | A | 1005 | 73.166 | 48.321 | −13.330 | 1.00 | 20.66 | A |
| ATOM | 7879 | CG | ASN | A | 1005 | 74.151 | 49.043 | −14.231 | 1.00 | 21.46 | A |
| ATOM | 7880 | OD1 | ASN | A | 1005 | 74.441 | 50.223 | −14.030 | 1.00 | 23.60 | A |
| ATOM | 7881 | ND2 | ASN | A | 1005 | 74.673 | 48.336 | −15.225 | 1.00 | 22.61 | A |
| ATOM | 7882 | C | ASN | A | 1005 | 71.156 | 46.857 | −13.163 | 1.00 | 19.34 | A |
| ATOM | 7883 | O | ASN | A | 1005 | 71.314 | 46.933 | −11.945 | 1.00 | 19.19 | A |
| ATOM | 7884 | N | VAL | A | 1006 | 70.087 | 46.291 | −13.713 | 1.00 | 19.06 | A |
| ATOM | 7885 | CA | VAL | A | 1006 | 69.036 | 45.723 | −12.876 | 1.00 | 19.31 | A |
| ATOM | 7886 | CB | VAL | A | 1006 | 67.747 | 45.434 | −13.693 | 1.00 | 19.18 | A |
| ATOM | 7887 | CG1 | VAL | A | 1006 | 68.019 | 44.404 | −14.773 | 1.00 | 19.81 | A |
| ATOM | 7888 | CG2 | VAL | A | 1006 | 66.639 | 44.957 | −12.764 | 1.00 | 19.49 | A |
| ATOM | 7889 | C | VAL | A | 1006 | 69.545 | 44.435 | −12.230 | 1.00 | 19.15 | A |
| ATOM | 7890 | O | VAL | A | 1006 | 70.094 | 43.563 | −12.906 | 1.00 | 19.87 | A |
| ATOM | 7891 | N | ALA | A | 1007 | 69.368 | 44.331 | −10.916 | 1.00 | 18.45 | A |
| ATOM | 7892 | CA | ALA | A | 1007 | 69.817 | 43.169 | −10.154 | 1.00 | 18.81 | A |
| ATOM | 7893 | CB | ALA | A | 1007 | 70.580 | 43.629 | −8.919 | 1.00 | 18.61 | A |
| ATOM | 7894 | C | ALA | A | 1007 | 68.652 | 42.272 | −9.748 | 1.00 | 19.17 | A |
| ATOM | 7895 | O | ALA | A | 1007 | 68.836 | 41.086 | −9.472 | 1.00 | 19.63 | A |
| ATOM | 7896 | N | ARG | A | 1008 | 67.455 | 42.844 | −9.695 | 1.00 | 19.17 | A |
| ATOM | 7897 | CA | ARG | A | 1008 | 66.265 | 42.078 | −9.349 | 1.00 | 19.29 | A |
| ATOM | 7898 | CB | ARG | A | 1008 | 66.261 | 41.696 | −7.863 | 1.00 | 22.65 | A |
| ATOM | 7899 | CG | ARG | A | 1008 | 66.156 | 42.852 | −6.896 | 1.00 | 27.30 | A |
| ATOM | 7900 | CD | ARG | A | 1008 | 66.010 | 42.347 | −5.466 | 1.00 | 31.10 | A |
| ATOM | 7901 | NE | ARG | A | 1008 | 67.134 | 41.503 | −5.065 | 1.00 | 34.46 | A |
| ATOM | 7902 | CZ | ARG | A | 1008 | 68.392 | 41.924 | −4.962 | 1.00 | 36.16 | A |
| ATOM | 7903 | NH1 | ARG | A | 1008 | 68.699 | 43.187 | −5.228 | 1.00 | 37.00 | A |
| ATOM | 7904 | NH2 | ARG | A | 1008 | 69.347 | 41.078 | −4.596 | 1.00 | 37.06 | A |
| ATOM | 7905 | C | ARG | A | 1008 | 65.003 | 42.855 | −9.688 | 1.00 | 17.93 | A |
| ATOM | 7906 | O | ARG | A | 1008 | 65.010 | 44.085 | −9.738 | 1.00 | 17.30 | A |
| ATOM | 7907 | N | CYS | A | 1009 | 63.927 | 42.118 | −9.933 | 1.00 | 16.65 | A |
| ATOM | 7908 | CA | CYS | A | 1009 | 62.636 | 42.698 | −10.276 | 1.00 | 16.67 | A |
| ATOM | 7909 | C | CYS | A | 1009 | 61.592 | 41.996 | −9.427 | 1.00 | 15.78 | A |
| ATOM | 7910 | O | CYS | A | 1009 | 61.519 | 40.768 | −9.412 | 1.00 | 15.09 | A |
| ATOM | 7911 | CB | CYS | A | 1009 | 62.339 | 42.464 | −11.753 | 1.00 | 18.24 | A |
| ATOM | 7912 | SG | CYS | A | 1009 | 60.816 | 43.242 | −12.385 | 1.00 | 21.28 | A |
| ATOM | 7913 | N | GLU | A | 1010 | 60.776 | 42.775 | −8.727 | 1.00 | 14.67 | A |
| ATOM | 7914 | CA | GLU | A | 1010 | 59.761 | 42.197 | −7.866 | 1.00 | 14.58 | A |
| ATOM | 7915 | CB | GLU | A | 1010 | 60.174 | 42.376 | −6.401 | 1.00 | 15.93 | A |
| ATOM | 7916 | CG | GLU | A | 1010 | 61.501 | 41.712 | −6.045 | 1.00 | 18.90 | A |
| ATOM | 7917 | CD | GLU | A | 1010 | 62.107 | 42.248 | −4.760 | 1.00 | 20.54 | A |
| ATOM | 7918 | OE1 | GLU | A | 1010 | 62.440 | 43.452 | −4.712 | 1.00 | 22.00 | A |
| ATOM | 7919 | OE2 | GLU | A | 1010 | 62.255 | 41.466 | −3.796 | 1.00 | 22.46 | A |
| ATOM | 7920 | C | GLU | A | 1010 | 58.394 | 42.827 | −8.075 | 1.00 | 13.40 | A |
| ATOM | 7921 | O | GLU | A | 1010 | 58.284 | 44.024 | −8.347 | 1.00 | 13.98 | A |
| ATOM | 7922 | N | ARG | A | 1011 | 57.354 | 42.009 | −7.970 | 1.00 | 12.76 | A |
| ATOM | 7923 | CA | ARG | A | 1011 | 55.991 | 42.512 | −8.072 | 1.00 | 12.10 | A |
| ATOM | 7924 | CB | ARG | A | 1011 | 55.034 | 41.430 | −8.573 | 1.00 | 13.80 | A |
| ATOM | 7925 | CG | ARG | A | 1011 | 53.614 | 41.929 | −8.812 | 1.00 | 18.17 | A |
| ATOM | 7926 | CD | ARG | A | 1011 | 52.618 | 40.774 | −8.865 | 1.00 | 22.69 | A |
| ATOM | 7927 | NE | ARG | A | 1011 | 52.950 | 39.783 | −9.883 | 1.00 | 27.58 | A |
| ATOM | 7928 | CZ | ARG | A | 1011 | 52.911 | 40.010 | −11.192 | 1.00 | 28.97 | A |
| ATOM | 7929 | NH1 | ARG | A | 1011 | 52.554 | 41.203 | −11.651 | 1.00 | 30.64 | A |
| ATOM | 7930 | NH2 | ARG | A | 1011 | 53.224 | 39.040 | −12.044 | 1.00 | 29.56 | A |
| ATOM | 7931 | C | ARG | A | 1011 | 55.666 | 42.853 | −6.621 | 1.00 | 11.53 | A |
| ATOM | 7932 | O | ARG | A | 1011 | 55.977 | 42.076 | −5.712 | 1.00 | 11.37 | A |
| ATOM | 7933 | N | THR | A | 1012 | 55.062 | 44.012 | −6.397 | 1.00 | 10.37 | A |
| ATOM | 7934 | CA | THR | A | 1012 | 54.734 | 44.446 | −5.045 | 1.00 | 9.85 | A |
| ATOM | 7935 | CB | THR | A | 1012 | 55.624 | 45.631 | −4.618 | 1.00 | 11.29 | A |
| ATOM | 7936 | OG1 | THR | A | 1012 | 55.297 | 46.777 | −5.421 | 1.00 | 10.95 | A |
| ATOM | 7937 | CG2 | THR | A | 1012 | 57.097 | 45.301 | −4.810 | 1.00 | 11.40 | A |
| ATOM | 7938 | C | THR | A | 1012 | 53.295 | 44.931 | −4.955 | 1.00 | 10.13 | A |
| ATOM | 7939 | O | THR | A | 1012 | 52.595 | 45.038 | −5.961 | 1.00 | 9.43 | A |
| ATOM | 7940 | N | THR | A | 1013 | 52.858 | 45.216 | −3.733 | 1.00 | 9.30 | A |
| ATOM | 7941 | CA | THR | A | 1013 | 51.532 | 45.772 | −3.522 | 1.00 | 9.02 | A |
| ATOM | 7942 | CB | THR | A | 1013 | 51.229 | 45.899 | −2.024 | 1.00 | 9.03 | A |
| ATOM | 7943 | OG1 | THR | A | 1013 | 52.399 | 46.365 | −1.344 | 1.00 | 8.79 | A |
| ATOM | 7944 | CG2 | THR | A | 1013 | 50.809 | 44.553 | −1.442 | 1.00 | 10.35 | A |
| ATOM | 7945 | C | THR | A | 1013 | 51.622 | 47.164 | −4.163 | 1.00 | 8.71 | A |
| ATOM | 7946 | O | THR | A | 1013 | 52.721 | 47.685 | −4.366 | 1.00 | 8.56 | A |
| ATOM | 7947 | N | LEU | A | 1014 | 50.484 | 47.776 | −4.469 | 1.00 | 8.27 | A |
| ATOM | 7948 | CA | LEU | A | 1014 | 50.504 | 49.082 | −5.133 | 1.00 | 7.70 | A |
| ATOM | 7949 | CB | LEU | A | 1014 | 49.085 | 49.520 | −5.491 | 1.00 | 7.86 | A |
| ATOM | 7950 | CG | LEU | A | 1014 | 48.309 | 48.581 | −6.414 | 1.00 | 7.61 | A |
| ATOM | 7951 | CD1 | LEU | A | 1014 | 47.005 | 49.264 | −6.789 | 1.00 | 8.33 | A |
| ATOM | 7952 | CD2 | LEU | A | 1014 | 49.116 | 48.242 | −7.667 | 1.00 | 8.06 | A |
| ATOM | 7953 | C | LEU | A | 1014 | 51.197 | 50.205 | −4.376 | 1.00 | 7.40 | A |
| ATOM | 7954 | O | LEU | A | 1014 | 51.552 | 51.228 | −4.964 | 1.00 | 7.36 | A |
| ATOM | 7955 | N | THR | A | 1015 | 51.382 | 50.011 | −3.076 | 1.00 | 7.72 | A |
| ATOM | 7956 | CA | THR | A | 1015 | 52.037 | 50.991 | −2.220 | 1.00 | 7.89 | A |
| ATOM | 7957 | CB | THR | A | 1015 | 51.511 | 50.885 | −0.794 | 1.00 | 8.20 | A |
| ATOM | 7958 | OG1 | THR | A | 1015 | 51.649 | 49.527 | −0.362 | 1.00 | 8.50 | A |
| ATOM | 7959 | CG2 | THR | A | 1015 | 50.045 | 51.287 | −0.724 | 1.00 | 8.31 | A |
| ATOM | 7960 | C | THR | A | 1015 | 53.544 | 50.741 | −2.166 | 1.00 | 8.46 | A |
| ATOM | 7961 | O | THR | A | 1015 | 54.274 | 51.501 | −1.528 | 1.00 | 9.16 | A |
| ATOM | 7962 | N | PHE | A | 1016 | 53.988 | 49.668 | −2.823 | 1.00 | 8.59 | A |
| ATOM | 7963 | CA | PHE | A | 1016 | 55.402 | 49.273 | −2.867 | 1.00 | 9.00 | A |
| ATOM | 7964 | CB | PHE | A | 1016 | 56.298 | 50.457 | −3.263 | 1.00 | 9.48 | A |
| ATOM | 7965 | CG | PHE | A | 1016 | 55.972 | 51.061 | −4.598 | 1.00 | 8.69 | A |
| ATOM | 7966 | CD1 | PHE | A | 1016 | 55.795 | 52.438 | −4.718 | 1.00 | 8.66 | A |
| ATOM | 7967 | CD2 | PHE | A | 1016 | 55.877 | 50.270 | −5.738 | 1.00 | 8.29 | A |
| ATOM | 7968 | CE1 | PHE | A | 1016 | 55.528 | 53.016 | −5.956 | 1.00 | 8.19 | A |
| ATOM | 7969 | CE2 | PHE | A | 1016 | 55.609 | 50.841 | −6.981 | 1.00 | 9.07 | A |
| ATOM | 7970 | CZ | PHE | A | 1016 | 55.435 | 52.213 | −7.089 | 1.00 | 9.64 | A |
| ATOM | 7971 | C | PHE | A | 1016 | 55.899 | 48.744 | −1.523 | 1.00 | 9.84 | A |
| ATOM | 7972 | O | PHE | A | 1016 | 57.089 | 48.470 | −1.368 | 1.00 | 10.75 | A |
| ATOM | 7973 | N | LEU | A | 1017 | 54.996 | 48.579 | −0.562 | 1.00 | 10.15 | A |
| ATOM | 7974 | CA | LEU | A | 1017 | 55.400 | 48.144 | 0.773 | 1.00 | 10.97 | A |
| ATOM | 7975 | CB | LEU | A | 1017 | 54.430 | 48.717 | 1.808 | 1.00 | 10.65 | A |
| ATOM | 7976 | CG | LEU | A | 1017 | 54.380 | 50.250 | 1.792 | 1.00 | 9.95 | A |
| ATOM | 7977 | CD1 | LEU | A | 1017 | 53.336 | 50.737 | 2.780 | 1.00 | 10.62 | A |
| ATOM | 7978 | CD2 | LEU | A | 1017 | 55.749 | 50.828 | 2.133 | 1.00 | 11.90 | A |
| ATOM | 7979 | C | LEU | A | 1017 | 55.607 | 46.655 | 1.024 | 1.00 | 11.87 | A |
| ATOM | 7980 | O | LEU | A | 1017 | 56.252 | 46.289 | 2.006 | 1.00 | 13.86 | A |
| ATOM | 7981 | N | GLN | A | 1018 | 55.066 | 45.794 | 0.169 | 1.00 | 12.59 | A |
| ATOM | 7982 | CA | GLN | A | 1018 | 55.262 | 44.359 | 0.352 | 1.00 | 14.35 | A |
| ATOM | 7983 | CB | GLN | A | 1018 | 54.001 | 43.689 | 0.912 | 1.00 | 16.37 | A |
| ATOM | 7984 | CG | GLN | A | 1018 | 54.197 | 42.192 | 1.161 | 1.00 | 19.20 | A |
| ATOM | 7985 | CD | GLN | A | 1018 | 52.916 | 41.454 | 1.503 | 1.00 | 21.24 | A |
| ATOM | 7986 | OE1 | GLN | A | 1018 | 52.925 | 40.236 | 1.694 | 1.00 | 23.07 | A |
| ATOM | 7987 | NE2 | GLN | A | 1018 | 51.809 | 42.182 | 1.578 | 1.00 | 21.22 | A |
| ATOM | 7988 | C | GLN | A | 1018 | 55.634 | 43.680 | −0.959 | 1.00 | 14.71 | A |
| ATOM | 7989 | O | GLN | A | 1018 | 55.018 | 43.933 | −1.995 | 1.00 | 14.64 | A |
| ATOM | 7990 | N | ASN | A | 1019 | 56.647 | 42.819 | −0.914 | 1.00 | 15.34 | A |
| ATOM | 7991 | CA | ASN | A | 1019 | 57.069 | 42.092 | −2.104 | 1.00 | 16.71 | A |
| ATOM | 7992 | CB | ASN | A | 1019 | 58.536 | 41.665 | −1.989 | 1.00 | 17.63 | A |
| ATOM | 7993 | CG | ASN | A | 1019 | 59.475 | 42.847 | −1.852 | 1.00 | 19.02 | A |
| ATOM | 7994 | OD1 | ASN | A | 1019 | 59.322 | 43.858 | −2.540 | 1.00 | 19.65 | A |
| ATOM | 7995 | ND2 | ASN | A | 1019 | 60.462 | 42.722 | −0.971 | 1.00 | 19.85 | A |
| ATOM | 7996 | C | ASN | A | 1019 | 56.178 | 40.867 | −2.253 | 1.00 | 17.34 | A |
| ATOM | 7997 | O | ASN | A | 1019 | 56.054 | 40.063 | −1.327 | 1.00 | 18.14 | A |
| ATOM | 7998 | N | LEU | A | 1020 | 55.564 | 40.727 | −3.422 | 1.00 | 17.74 | A |
| ATOM | 7999 | CA | LEU | A | 1020 | 54.657 | 39.618 | −3.685 | 1.00 | 18.66 | A |
| ATOM | 8000 | CB | LEU | A | 1020 | 53.373 | 40.144 | −4.331 | 1.00 | 18.41 | A |
| ATOM | 8001 | CG | LEU | A | 1020 | 52.582 | 41.198 | −3.551 | 1.00 | 17.65 | A |
| ATOM | 8002 | CD1 | LEU | A | 1020 | 51.433 | 41.703 | −4.405 | 1.00 | 18.32 | A |
| ATOM | 8003 | CD2 | LEU | A | 1020 | 52.064 | 40.602 | −2.251 | 1.00 | 17.91 | A |
| ATOM | 8004 | C | LEU | A | 1020 | 55.249 | 38.527 | −4.571 | 1.00 | 19.82 | A |
| ATOM | 8005 | O | LEU | A | 1020 | 54.851 | 37.363 | −4.474 | 1.00 | 20.37 | A |
| ATOM | 8006 | N | GLU | A | 1021 | 56.190 | 38.900 | −5.432 | 1.00 | 20.85 | A |
| ATOM | 8007 | CA | GLU | A | 1021 | 56.808 | 37.942 | −6.347 | 1.00 | 22.64 | A |
| ATOM | 8008 | CB | GLU | A | 1021 | 55.947 | 37.778 | −7.601 | 1.00 | 24.55 | A |
| ATOM | 8009 | CG | GLU | A | 1021 | 54.627 | 37.066 | −7.415 | 1.00 | 27.54 | A |
| ATOM | 8010 | CD | GLU | A | 1021 | 53.845 | 36.990 | −8.713 | 1.00 | 29.04 | A |
| ATOM | 8011 | OE1 | GLU | A | 1021 | 54.450 | 36.632 | −9.746 | 1.00 | 29.72 | A |
| ATOM | 8012 | OE2 | GLU | A | 1021 | 52.631 | 37.283 | −8.701 | 1.00 | 30.41 | A |
| ATOM | 8013 | C | GLU | A | 1021 | 58.207 | 38.331 | −6.804 | 1.00 | 22.88 | A |
| ATOM | 8014 | O | GLU | A | 1021 | 58.474 | 39.494 | −7.100 | 1.00 | 21.59 | A |
| ATOM | 8015 | N | HIS | A | 1022 | 59.093 | 37.343 | −6.870 | 1.00 | 23.24 | A |
| ATOM | 8016 | CA | HIS | A | 1022 | 60.451 | 37.559 | −7.348 | 1.00 | 24.68 | A |
| ATOM | 8017 | CB | HIS | A | 1022 | 61.438 | 36.663 | −6.595 | 1.00 | 25.87 | A |
| ATOM | 8018 | CG | HIS | A | 1022 | 62.860 | 36.831 | −7.032 | 1.00 | 27.45 | A |
| ATOM | 8019 | CD2 | HIS | A | 1022 | 83.768 | 35.930 | −7.477 | 1.00 | 28.54 | A |
| ATOM | 8020 | ND1 | HIS | A | 1022 | 63.500 | 38.052 | −7.033 | 1.00 | 28.74 | A |
| ATOM | 8021 | CE1 | HIS | A | 1022 | 64.740 | 37.896 | −7.460 | 1.00 | 29.03 | A |
| ATOM | 8022 | NE2 | HIS | A | 1022 | 64.929 | 36.618 | −7.736 | 1.00 | 29.19 | A |
| ATOM | 8023 | C | HIS | A | 1022 | 60.359 | 37.146 | −8.813 | 1.00 | 24.90 | A |
| ATOM | 8024 | O | HIS | A | 1022 | 60.105 | 35.982 | −9.122 | 1.00 | 25.14 | A |
| ATOM | 8025 | N | LEU | A | 1023 | 60.549 | 38.104 | −9.711 | 1.00 | 25.03 | A |
| ATOM | 8026 | CA | LEU | A | 1023 | 60.432 | 37.846 | −11.140 | 1.00 | 26.27 | A |
| ATOM | 8027 | CB | LEU | A | 1023 | 59.975 | 39.124 | −11.843 | 1.00 | 25.95 | A |
| ATOM | 8028 | CG | LEU | A | 1023 | 58.679 | 39.705 | −11.267 | 1.00 | 25.51 | A |
| ATOM | 8029 | CD1 | LEU | A | 1023 | 58.382 | 41.052 | −11.901 | 1.00 | 25.87 | A |
| ATOM | 8030 | CD2 | LEU | A | 1023 | 57.537 | 38.728 | −11.506 | 1.00 | 26.07 | A |
| ATOM | 8031 | C | LEU | A | 1023 | 61.685 | 37.303 | −11.816 | 1.00 | 27.30 | A |
| ATOM | 8032 | O | LEU | A | 1023 | 62.747 | 37.924 | −11.778 | 1.00 | 26.61 | A |
| ATOM | 8033 | N | ASP | A | 1024 | 61.546 | 36.139 | −12.443 | 1.00 | 29.41 | A |
| ATOM | 8034 | CA | ASP | A | 1024 | 62.659 | 35.509 | −13.143 | 1.00 | 31.40 | A |
| ATOM | 8035 | CB | ASP | A | 1024 | 62.283 | 34.091 | −13.582 | 1.00 | 33.04 | A |
| ATOM | 8036 | CG | ASP | A | 1024 | 62.251 | 33.111 | −12.424 | 1.00 | 34.34 | A |
| ATOM | 8037 | OD1 | ASP | A | 1024 | 61.961 | 31.919 | −12.661 | 1.00 | 35.68 | A |
| ATOM | 8038 | OD2 | ASP | A | 1024 | 62.521 | 33.529 | −11.278 | 1.00 | 35.28 | A |
| ATOM | 8039 | C | ASP | A | 1024 | 63.049 | 36.336 | −14.358 | 1.00 | 32.07 | A |
| ATOM | 8040 | O | ASP | A | 1024 | 62.202 | 36.977 | −14.985 | 1.00 | 32.21 | A |
| ATOM | 8041 | N | GLY | A | 1025 | 64.336 | 36.318 | −14.686 | 1.00 | 32.46 | A |
| ATOM | 8042 | CA | GLY | A | 1025 | 64.816 | 37.080 | −15.821 | 1.00 | 33.39 | A |
| ATOM | 8043 | C | GLY | A | 1025 | 64.726 | 38.564 | −15.534 | 1.00 | 33.83 | A |
| ATOM | 8044 | O | GLY | A | 1025 | 65.055 | 39.392 | −16.382 | 1.00 | 33.62 | A |
| ATOM | 8045 | N | MSE | A | 1026 | 64.277 | 38.894 | −14.326 | 1.00 | 34.40 | A |
| ATOM | 8046 | CA | MSE | A | 1026 | 64.133 | 40.281 | −13.904 | 1.00 | 35.14 | A |
| ATOM | 8047 | CB | MSE | A | 1026 | 65.504 | 40.943 | −13.809 | 1.00 | 38.95 | A |
| ATOM | 8048 | CG | MSE | A | 1026 | 66.467 | 40.210 | −12.900 | 1.00 | 43.37 | A |
| ATOM | 8049 | SE | MSE | A | 1026 | 68.190 | 41.048 | −12.869 | 1.00 | 48.57 | A |
| ATOM | 8050 | CE | MSE | A | 1026 | 68.959 | 40.193 | −14.422 | 1.00 | 46.31 | A |
| ATOM | 8051 | C | MSE | A | 1026 | 63.260 | 41.041 | −14.890 | 1.00 | 33.31 | A |
| ATOM | 8052 | O | MSE | A | 1026 | 63.493 | 42.218 | −15.166 | 1.00 | 32.52 | A |
| ATOM | 8053 | N | VAL | A | 1027 | 62.254 | 40.358 | −15.420 | 1.00 | 31.46 | A |
| ATOM | 8054 | CA | VAL | A | 1027 | 61.348 | 40.965 | −16.380 | 1.00 | 30.23 | A |
| ATOM | 8055 | CB | VAL | A | 1027 | 61.201 | 40.088 | −17.639 | 1.00 | 30.01 | A |
| ATOM | 8056 | CG1 | VAL | A | 1027 | 60.185 | 40.706 | −18.591 | 1.00 | 29.55 | A |
| ATOM | 8057 | CG2 | VAL | A | 1027 | 62.547 | 39.942 | −18.326 | 1.00 | 29.41 | A |
| ATOM | 8058 | C | VAL | A | 1027 | 59.969 | 41.185 | −15.778 | 1.00 | 29.79 | A |
| ATOM | 8059 | O | VAL | A | 1027 | 59.340 | 40.252 | −15.280 | 1.00 | 29.23 | A |
| ATOM | 8060 | N | ALA | A | 1028 | 59.508 | 42.430 | −15.821 | 1.00 | 28.92 | A |
| ATOM | 8061 | CA | ALA | A | 1028 | 58.194 | 42.773 | −15.298 | 1.00 | 28.80 | A |
| ATOM | 8062 | CB | ALA | A | 1028 | 58.184 | 44.213 | −14.799 | 1.00 | 28.67 | A |
| ATOM | 8063 | C | ALA | A | 1028 | 57.189 | 42.600 | −16.428 | 1.00 | 28.40 | A |
| ATOM | 8064 | O | ALA | A | 1028 | 57.167 | 43.387 | −17.373 | 1.00 | 28.55 | A |
| ATOM | 8065 | N | PRO | A | 1029 | 56.354 | 41.554 | −16.353 | 1.00 | 27.78 | A |
| ATOM | 8066 | CD | PRO | A | 1029 | 56.301 | 40.519 | −15.304 | 1.00 | 27.81 | A |
| ATOM | 8067 | CA | PRO | A | 1029 | 55.350 | 41.298 | −17.390 | 1.00 | 27.71 | A |
| ATOM | 8068 | CB | PRO | A | 1029 | 54.858 | 39.895 | −17.047 | 1.00 | 28.01 | A |
| ATOM | 8069 | CG | PRO | A | 1029 | 54.958 | 39.870 | −15.559 | 1.00 | 28.02 | A |
| ATOM | 8070 | C | PRO | A | 1029 | 54.226 | 42.329 | −17.378 | 1.00 | 27.12 | A |
| ATOM | 8071 | O | PRO | A | 1029 | 53.868 | 42.859 | −16.327 | 1.00 | 27.82 | A |
| ATOM | 8072 | N | GLU | A | 1030 | 53.674 | 42.618 | −18.551 | 1.00 | 26.11 | A |
| ATOM | 8073 | CA | GLU | A | 1030 | 52.591 | 43.585 | −18.643 | 1.00 | 24.92 | A |
| ATOM | 8074 | CB | GLU | A | 1030 | 52.275 | 43.902 | −20.106 | 1.00 | 25.04 | A |
| ATOM | 8075 | CG | GLU | A | 1030 | 53.484 | 44.338 | −20.920 | 1.00 | 25.00 | A |
| ATOM | 8076 | CD | GLU | A | 1030 | 53.107 | 44.817 | −22.307 | 1.00 | 25.45 | A |
| ATOM | 8077 | OE1 | GLU | A | 1030 | 52.177 | 44.237 | −22.903 | 1.00 | 26.43 | A |
| ATOM | 8078 | OE2 | GLU | A | 1030 | 53.749 | 45.765 | −22.806 | 1.00 | 24.53 | A |
| ATOM | 8079 | C | GLU | A | 1030 | 51.357 | 43.019 | −17.953 | 1.00 | 23.99 | A |
| ATOM | 8080 | O | GLU | A | 1030 | 51.256 | 41.810 | −17.731 | 1.00 | 25.08 | A |
| ATOM | 8081 | N | VAL | A | 1031 | 50.418 | 43.896 | −17.618 | 1.00 | 22.66 | A |
| ATOM | 8082 | CA | VAL | A | 1031 | 49.204 | 43.480 | −16.933 | 1.00 | 20.61 | A |
| ATOM | 8083 | CB | VAL | A | 1031 | 48.801 | 44.515 | −15.860 | 1.00 | 21.38 | A |
| ATOM | 8084 | CG1 | VAL | A | 1031 | 49.925 | 44.673 | −14.851 | 1.00 | 21.76 | A |
| ATOM | 8085 | CG2 | VAL | A | 1031 | 48.487 | 45.847 | −16.508 | 1.00 | 20.47 | A |
| ATOM | 8086 | C | VAL | A | 1031 | 48.022 | 43.257 | −17.869 | 1.00 | 19.15 | A |
| ATOM | 8087 | O | VAL | A | 1031 | 48.063 | 43.621 | −19.044 | 1.00 | 19.47 | A |
| ATOM | 8088 | N | CYS | A | 1032 | 46.974 | 42.649 | −17.326 | 1.00 | 17.67 | A |
| ATOM | 8089 | CA | CYS | A | 1032 | 45.755 | 42.352 | −18.069 | 1.00 | 16.55 | A |
| ATOM | 8090 | C | CYS | A | 1032 | 44.838 | 43.566 | −18.141 | 1.00 | 14.35 | A |
| ATOM | 8091 | O | CYS | A | 1032 | 45.002 | 44.520 | −17.384 | 1.00 | 12.80 | A |
| ATOM | 8092 | CB | CYS | A | 1032 | 44.989 | 41.225 | −17.384 | 1.00 | 17.91 | A |
| ATOM | 8093 | SG | CYS | A | 1032 | 45.736 | 39.571 | −17.486 | 1.00 | 21.70 | A |
| ATOM | 8094 | N | PRO | A | 1033 | 43.854 | 43.544 | −19.056 | 1.00 | 13.48 | A |
| ATOM | 8095 | CD | PRO | A | 1033 | 43.599 | 42.542 | −20.108 | 1.00 | 13.27 | A |
| ATOM | 8096 | CA | PRO | A | 1033 | 42.927 | 44.672 | −19.176 | 1.00 | 12.75 | A |
| ATOM | 8097 | CB | PRO | A | 1033 | 41.964 | 44.212 | −20.270 | 1.00 | 13.14 | A |
| ATOM | 8098 | CG | PRO | A | 1033 | 42.837 | 43.346 | −21.136 | 1.00 | 13.23 | A |
| ATOM | 8099 | C | PRO | A | 1033 | 42.210 | 44.914 | −17.844 | 1.00 | 12.09 | A |
| ATOM | 8100 | O | PRO | A | 1033 | 41.721 | 43.974 | −17.209 | 1.00 | 11.14 | A |
| ATOM | 8101 | N | MSE | A | 1034 | 42.161 | 46.181 | −17.442 | 1.00 | 11.64 | A |
| ATOM | 8102 | CA | MSE | A | 1034 | 41.531 | 46.631 | −16.201 | 1.00 | 12.45 | A |
| ATOM | 8103 | CB | MSE | A | 1034 | 40.099 | 46.099 | −16.084 | 1.00 | 12.75 | A |
| ATOM | 8104 | CG | MSE | A | 1034 | 39.156 | 46.629 | −17.161 | 1.00 | 13.48 | A |
| ATOM | 8105 | SE | MSE | A | 1034 | 39.229 | 48.559 | −17.394 | 1.00 | 18.29 | A |
| ATOM | 8106 | CE | MSE | A | 1034 | 38.290 | 49.085 | −15.785 | 1.00 | 15.04 | A |
| ATOM | 8107 | C | MSE | A | 1034 | 42.320 | 46.266 | −14.950 | 1.00 | 13.04 | A |
| ATOM | 8108 | O | MSE | A | 1034 | 41.844 | 46.460 | −13.833 | 1.00 | 15.57 | A |
| ATOM | 8109 | N | GLU | A | 1035 | 43.530 | 45.753 | −15.139 | 1.00 | 12.47 | A |
| ATOM | 8110 | CA | GLU | A | 1035 | 44.379 | 45.385 | −14.016 | 1.00 | 12.77 | A |
| ATOM | 8111 | CB | GLU | A | 1035 | 45.068 | 44.040 | −14.282 | 1.00 | 15.80 | A |
| ATOM | 8112 | CG | GLU | A | 1035 | 46.248 | 43.751 | −13.359 | 1.00 | 20.78 | A |
| ATOM | 8113 | CD | GLU | A | 1035 | 46.741 | 42.316 | −13.450 | 1.00 | 22.65 | A |
| ATOM | 8114 | OE1 | GLU | A | 1035 | 47.035 | 41.843 | −14.571 | 1.00 | 23.74 | A |
| ATOM | 8115 | OE2 | GLU | A | 1035 | 46.841 | 41.661 | −12.390 | 1.00 | 24.85 | A |
| ATOM | 8116 | C | GLU | A | 1035 | 45.429 | 46.460 | −13.773 | 1.00 | 11.49 | A |
| ATOM | 8117 | O | GLU | A | 1035 | 45.795 | 47.205 | −14.680 | 1.00 | 10.22 | A |
| ATOM | 8118 | N | THR | A | 1036 | 45.893 | 46.536 | −12.531 | 1.00 | 10.28 | A |
| ATOM | 8119 | CA | THR | A | 1036 | 46.919 | 47.483 | −12.125 | 1.00 | 9.92 | A |
| ATOM | 8120 | CB | THR | A | 1036 | 46.343 | 48.607 | −11.245 | 1.00 | 9.77 | A |
| ATOM | 8121 | OG1 | THR | A | 1036 | 45.278 | 49.264 | −11.937 | 1.00 | 10.21 | A |
| ATOM | 8122 | CG2 | THR | A | 1036 | 47.418 | 49.629 | −10.914 | 1.00 | 10.57 | A |
| ATOM | 8123 | C | THR | A | 1036 | 47.906 | 46.678 | −11.293 | 1.00 | 9.13 | A |
| ATOM | 8124 | O | THR | A | 1036 | 47.502 | 45.935 | −10.400 | 1.00 | 10.16 | A |
| ATOM | 8125 | N | ALA | A | 1037 | 49.192 | 46.817 | −11.594 | 1.00 | 9.44 | A |
| ATOM | 8126 | CA | ALA | A | 1037 | 50.225 | 46.096 | −10.865 | 1.00 | 9.72 | A |
| ATOM | 8127 | CB | ALA | A | 1037 | 50.747 | 44.937 | −11.705 | 1.00 | 10.95 | A |
| ATOM | 8128 | C | ALA | A | 1037 | 51.361 | 47.046 | −10.537 | 1.00 | 9.84 | A |
| ATOM | 8129 | O | ALA | A | 1037 | 51.516 | 48.081 | −11.177 | 1.00 | 10.76 | A |
| ATOM | 8130 | N | ALA | A | 1038 | 52.152 | 46.693 | −9.533 | 1.00 | 9.36 | A |
| ATOM | 8131 | CA | ALA | A | 1038 | 53.283 | 47.515 | −9.144 | 1.00 | 8.90 | A |
| ATOM | 8132 | CB | ALA | A | 1038 | 53.073 | 48.080 | −7.747 | 1.00 | 8.87 | A |
| ATOM | 8133 | C | ALA | A | 1038 | 54.534 | 46.656 | −9.180 | 1.00 | 8.90 | A |
| ATOM | 8134 | O | ALA | A | 1038 | 54.497 | 45.481 | −8.813 | 1.00 | 7.98 | A |
| ATOM | 8135 | N | TYR | A | 1039 | 55.634 | 47.245 | −9.635 | 1.00 | 9.04 | A |
| ATOM | 8136 | CA | TYR | A | 1039 | 56.903 | 46.540 | −9.707 | 1.00 | 10.18 | A |
| ATOM | 8137 | CB | TYR | A | 1039 | 57.248 | 46.177 | −11.151 | 1.00 | 10.53 | A |
| ATOM | 8138 | CG | TYR | A | 1039 | 56.243 | 45.288 | −11.837 | 1.00 | 11.78 | A |
| ATOM | 8139 | CD1 | TYR | A | 1039 | 55.324 | 45.813 | −12.746 | 1.00 | 12.61 | A |
| ATOM | 8140 | CE1 | TYR | A | 1039 | 54.405 | 44.990 | −13.394 | 1.00 | 14.80 | A |
| ATOM | 8141 | CD2 | TYR | A | 1039 | 56.216 | 43.918 | −11.589 | 1.00 | 12.35 | A |
| ATOM | 8142 | CE2 | TYR | A | 1039 | 55.301 | 43.089 | −12.228 | 1.00 | 13.71 | A |
| ATOM | 8143 | CZ | TYR | A | 1039 | 54.401 | 43.632 | −13.131 | 1.00 | 15.21 | A |
| ATOM | 8144 | OH | TYR | A | 1039 | 53.508 | 42.815 | −13.783 | 1.00 | 17.85 | A |
| ATOM | 8145 | C | TYR | A | 1039 | 58.009 | 47.422 | −9.164 | 1.00 | 10.60 | A |
| ATOM | 8146 | O | TYR | A | 1039 | 57.950 | 48.644 | −9.269 | 1.00 | 11.19 | A |
| ATOM | 8147 | N | VAL | A | 1040 | 59.019 | 46.795 | −8.575 | 1.00 | 10.57 | A |
| ATOM | 8148 | CA | VAL | A | 1040 | 60.158 | 47.529 | −8.058 | 1.00 | 10.83 | A |
| ATOM | 8149 | CB | VAL | A | 1040 | 60.223 | 47.503 | −6.513 | 1.00 | 10.80 | A |
| ATOM | 8150 | CG1 | VAL | A | 1040 | 61.527 | 48.144 | −6.034 | 1.00 | 10.81 | A |
| ATOM | 8151 | CG2 | VAL | A | 1040 | 59.039 | 48.259 | −5.934 | 1.00 | 11.19 | A |
| ATOM | 8152 | C | VAL | A | 1040 | 61.414 | 46.884 | −8.615 | 1.00 | 11.68 | A |
| ATOM | 8153 | O | VAL | A | 1040 | 61.634 | 45.681 | −8.454 | 1.00 | 12.74 | A |
| ATOM | 8154 | N | SER | A | 1041 | 62.221 | 47.680 | −9.303 | 1.00 | 11.34 | A |
| ATOM | 8155 | CA | SER | A | 1041 | 63.465 | 47.174 | −9.855 | 1.00 | 12.00 | A |
| ATOM | 8156 | CB | SER | A | 1041 | 63.679 | 47.700 | −11.281 | 1.00 | 12.02 | A |
| ATOM | 8157 | OG | SER | A | 1041 | 63.674 | 49.114 | −11.326 | 1.00 | 13.73 | A |
| ATOM | 8158 | C | SER | A | 1041 | 64.600 | 47.627 | −8.947 | 1.00 | 12.06 | A |
| ATOM | 8159 | O | SER | A | 1041 | 64.608 | 48.759 | −8.464 | 1.00 | 11.83 | A |
| ATOM | 8160 | N | SER | A | 1042 | 65.544 | 46.726 | −8.695 | 1.00 | 12.11 | A |
| ATOM | 8161 | CA | SER | A | 1042 | 66.696 | 47.038 | −7.858 | 1.00 | 12.84 | A |
| ATOM | 8162 | CB | SER | A | 1042 | 66.899 | 45.959 | −6.793 | 1.00 | 11.51 | A |
| ATOM | 8163 | OG | SER | A | 1042 | 65.783 | 45.904 | −5.918 | 1.00 | 14.87 | A |
| ATOM | 8164 | C | SER | A | 1042 | 67.906 | 47.117 | −8.772 | 1.00 | 13.04 | A |
| ATOM | 8165 | O | SER | A | 1042 | 68.061 | 46.301 | −9.682 | 1.00 | 14.12 | A |
| ATOM | 8166 | N | HIS | A | 1043 | 68.757 | 48.106 | −8.530 | 1.00 | 14.49 | A |
| ATOM | 8167 | CA | HIS | A | 1043 | 69.931 | 48.318 | −9.363 | 1.00 | 15.99 | A |
| ATOM | 8168 | CB | HIS | A | 1043 | 69.709 | 49.582 | −10.193 | 1.00 | 15.32 | A |
| ATOM | 8169 | CG | HIS | A | 1043 | 68.402 | 49.585 | −10.922 | 1.00 | 14.89 | A |
| ATOM | 8170 | CD2 | HIS | A | 1043 | 67.158 | 49.931 | −10.514 | 1.00 | 13.91 | A |
| ATOM | 8171 | ND1 | HIS | A | 1043 | 68.260 | 49.088 | −12.199 | 1.00 | 14.17 | A |
| ATOM | 8172 | CE1 | HIS | A | 1043 | 66.986 | 49.125 | −12.547 | 1.00 | 15.10 | A |
| ATOM | 8173 | NE2 | HIS | A | 1043 | 66.296 | 49.631 | −11.540 | 1.00 | 14.41 | A |
| ATOM | 8174 | C | HIS | A | 1043 | 71.199 | 48.425 | −8.530 | 1.00 | 17.89 | A |
| ATOM | 8175 | O | HIS | A | 1043 | 71.251 | 49.156 | −7.541 | 1.00 | 17.26 | A |
| ATOM | 8176 | N | SER | A | 1044 | 72.220 | 47.684 | −8.943 | 1.00 | 20.49 | A |
| ATOM | 8177 | CA | SER | A | 1044 | 73.493 | 47.666 | −8.237 | 1.00 | 23.36 | A |
| ATOM | 8178 | CB | SER | A | 1044 | 74.196 | 46.327 | −8.480 | 1.00 | 23.82 | A |
| ATOM | 8179 | OG | SER | A | 1044 | 74.218 | 46.009 | −9.862 | 1.00 | 25.95 | A |
| ATOM | 8180 | C | SER | A | 1044 | 74.404 | 48.813 | −8.649 | 1.00 | 24.24 | A |
| ATOM | 8181 | O | SER | A | 1044 | 74.018 | 49.585 | −9.554 | 1.00 | 24.99 | A |
| ATOM | 8182 | OXT | SER | A | 1044 | 75.497 | 48.922 | −8.053 | 1.00 | 25.64 | A |
| ATOM | 8183 | OH2 | WAT | W | 2 | 42.042 | 63.477 | −7.164 | 1.00 | 6.41 | W |
| ATOM | 8184 | OH2 | WAT | W | 3 | 53.550 | 64.836 | −19.873 | 1.00 | 8.14 | W |
| ATOM | 8185 | OH2 | WAT | W | 4 | 39.197 | 62.878 | −19.106 | 1.00 | 7.97 | W |
| ATOM | 8186 | OH2 | WAT | W | 5 | 52.209 | 54.085 | −4.846 | 1.00 | 8.40 | W |
| ATOM | 8187 | OH2 | WAT | W | 6 | 56.128 | 53.417 | −0.630 | 1.00 | 8.30 | W |
| ATOM | 8188 | OH2 | WAT | W | 7 | 31.282 | 49.937 | −23.972 | 1.00 | 10.14 | W |
| ATOM | 8189 | OH2 | WAT | W | 8 | 49.807 | 48.483 | 1.372 | 1.00 | 9.31 | W |
| ATOM | 8190 | OH2 | WAT | W | 9 | 36.893 | 57.648 | 13.387 | 1.00 | 9.61 | W |
| ATOM | 8191 | OH2 | WAT | W | 10 | 26.802 | 68.971 | −9.206 | 1.00 | 9.53 | W |
| ATOM | 8192 | OH2 | WAT | W | 11 | 39.233 | 64.818 | −14.778 | 1.00 | 10.18 | W |
| ATOM | 8193 | OH2 | WAT | W | 12 | 34.214 | 58.561 | −8.785 | 1.00 | 7.85 | W |
| ATOM | 8194 | OH2 | WAT | W | 13 | 61.037 | 59.682 | −8.191 | 1.00 | 8.41 | W |
| ATOM | 8195 | OH2 | WAT | W | 14 | 36.556 | 72.572 | 0.269 | 1.00 | 10.25 | W |
| ATOM | 8196 | OH2 | WAT | W | 15 | 31.717 | 47.036 | −8.074 | 1.00 | 8.83 | W |
| ATOM | 8197 | OH2 | WAT | W | 16 | 30.129 | 55.760 | 17.242 | 1.00 | 9.77 | W |
| ATOM | 8198 | OH2 | WAT | W | 17 | 26.161 | 49.593 | −13.680 | 1.00 | 9.92 | W |
| ATOM | 8199 | OH2 | WAT | W | 18 | 37.670 | 52.088 | −21.093 | 1.00 | 11.68 | W |
| ATOM | 8200 | OH2 | WAT | W | 19 | 33.074 | 62.758 | 0.070 | 1.00 | 7.76 | W |
| ATOM | 8201 | OH2 | WAT | W | 20 | 24.794 | 52.920 | −11.748 | 1.00 | 8.84 | W |
| ATOM | 8202 | OH2 | WAT | W | 21 | 63.100 | 61.310 | −7.515 | 1.00 | 9.52 | W |
| ATOM | 8203 | OH2 | WAT | W | 22 | 41.220 | 59.000 | 13.470 | 1.00 | 11.28 | W |
| ATOM | 8204 | OH2 | WAT | W | 23 | 47.214 | 55.792 | −15.391 | 1.00 | 16.11 | W |
| ATOM | 8205 | OH2 | WAT | W | 24 | 56.347 | 55.690 | −2.127 | 1.00 | 9.59 | W |
| ATOM | 8206 | OH2 | WAT | W | 25 | 67.413 | 60.727 | −5.662 | 1.00 | 12.47 | W |
| ATOM | 8207 | OH2 | WAT | W | 26 | 26.340 | 48.895 | −10.998 | 1.00 | 9.95 | W |
| ATOM | 8208 | OH2 | WAT | W | 27 | 65.658 | 60.301 | −7.681 | 1.00 | 9.86 | W |
| ATOM | 8209 | OH2 | WAT | W | 28 | 32.556 | 60.049 | −1.797 | 1.00 | 9.32 | W |
| ATOM | 8210 | OH2 | WAT | W | 29 | 20.112 | 54.723 | 16.205 | 1.00 | 11.76 | W |
| ATOM | 8211 | OH2 | WAT | W | 30 | 23.748 | 55.505 | −23.327 | 1.00 | 10.76 | W |
| ATOM | 8212 | OH2 | WAT | W | 31 | 39.332 | 57.340 | 14.891 | 1.00 | 9.99 | W |
| ATOM | 8213 | OH2 | WAT | W | 32 | 20.372 | 58.804 | −21.924 | 1.00 | 10.68 | W |
| ATOM | 8214 | OH2 | WAT | W | 33 | 28.005 | 60.998 | 19.501 | 1.00 | 12.43 | W |
| ATOM | 8215 | OH2 | WAT | W | 34 | 34.100 | 56.345 | −26.283 | 1.00 | 8.61 | W |
| ATOM | 8216 | OH2 | WAT | W | 35 | 26.313 | 40.065 | 7.967 | 1.00 | 12.45 | W |
| ATOM | 8217 | OH2 | WAT | W | 36 | 63.802 | 50.178 | −14.014 | 1.00 | 12.29 | W |
| ATOM | 8218 | OH2 | WAT | W | 37 | 37.571 | 56.991 | 1.737 | 1.00 | 9.76 | W |
| ATOM | 8219 | OH2 | WAT | W | 38 | 24.021 | 41.933 | 6.905 | 1.00 | 11.36 | W |
| ATOM | 8220 | OH2 | WAT | W | 39 | 31.878 | 65.446 | 18.975 | 1.00 | 15.13 | W |
| ATOM | 8221 | OH2 | WAT | W | 40 | 51.480 | 56.691 | −5.611 | 1.00 | 9.26 | W |
| ATOM | 8222 | OH2 | WAT | W | 41 | 20.134 | 56.063 | 7.164 | 1.00 | 12.78 | W |
| ATOM | 8223 | OH2 | WAT | W | 42 | 28.304 | 43.888 | 13.163 | 1.00 | 9.88 | W |
| ATOM | 8224 | OH2 | WAT | W | 43 | 30.243 | 58.465 | −11.811 | 1.00 | 8.09 | W |
| ATOM | 8225 | OH2 | WAT | W | 44 | 64.815 | 59.321 | −3.585 | 1.00 | 13.31 | W |
| ATOM | 8226 | OH2 | WAT | W | 45 | 46.389 | 67.686 | −15.597 | 1.00 | 13.23 | W |
| ATOM | 8227 | OH2 | WAT | W | 46 | 60.782 | 58.212 | −2.813 | 1.00 | 11.20 | W |
| ATOM | 8228 | OH2 | WAT | W | 47 | 60.514 | 62.158 | −1.234 | 1.00 | 13.28 | W |
| ATOM | 8229 | OH2 | WAT | W | 48 | 53.646 | 59.923 | −9.348 | 1.00 | 11.25 | W |
| ATOM | 8230 | OH2 | WAT | W | 49 | 18.643 | 51.090 | −12.404 | 1.00 | 11.27 | W |
| ATOM | 8231 | OH2 | WAT | W | 50 | 28.831 | 42.792 | −11.918 | 1.00 | 11.63 | W |
| ATOM | 8232 | OH2 | WAT | W | 51 | 34.530 | 79.088 | −9.168 | 1.00 | 11.13 | W |
| ATOM | 8233 | OH2 | WAT | W | 52 | 21.612 | 57.986 | −7.797 | 1.00 | 10.97 | W |
| ATOM | 8234 | OH2 | WAT | W | 53 | 37.026 | 71.830 | −4.514 | 1.00 | 9.38 | W |
| ATOM | 8235 | OH2 | WAT | W | 54 | 55.613 | 59.382 | −11.500 | 1.00 | 8.74 | W |
| ATOM | 8236 | OH2 | WAT | W | 55 | 36.685 | 44.923 | −5.472 | 1.00 | 11.75 | W |
| ATOM | 8237 | OH2 | WAT | W | 56 | 51.323 | 53.328 | −23.928 | 1.00 | 12.92 | W |
| ATOM | 8238 | OH2 | WAT | W | 57 | 35.162 | 50.576 | −17.524 | 1.00 | 13.57 | W |
| ATOM | 8239 | OH2 | WAT | W | 58 | 60.531 | 54.758 | −0.381 | 1.00 | 14.45 | W |
| ATOM | 8240 | OH2 | WAT | W | 59 | 42.905 | 56.765 | 6.206 | 1.00 | 11.63 | W |
| ATOM | 8241 | OH2 | WAT | W | 60 | 47.859 | 59.294 | −25.663 | 1.00 | 13.35 | W |
| ATOM | 8242 | OH2 | WAT | W | 61 | 37.863 | 73.312 | −2.170 | 1.00 | 12.61 | W |
| ATOM | 8243 | OH2 | WAT | W | 62 | 19.405 | 55.246 | 11.583 | 1.00 | 14.08 | W |
| ATOM | 8244 | OH2 | WAT | W | 63 | 25.931 | 39.712 | 5.244 | 1.00 | 11.23 | W |
| ATOM | 8245 | OH2 | WAT | W | 64 | 23.345 | 54.021 | −3.621 | 1.00 | 13.72 | W |
| ATOM | 8246 | OH2 | WAT | W | 65 | 34.262 | 52.377 | −0.944 | 1.00 | 9.34 | W |
| ATOM | 8247 | OH2 | WAT | W | 66 | 51.020 | 44.292 | −8.032 | 1.00 | 10.74 | W |
| ATOM | 8248 | OH2 | WAT | W | 67 | 37.640 | 58.989 | 11.075 | 1.00 | 12.29 | W |
| ATOM | 8249 | OH2 | WAT | W | 68 | 16.554 | 49.007 | 24.070 | 1.00 | 15.16 | W |
| ATOM | 8250 | OH2 | WAT | W | 69 | 19.376 | 53.552 | 8.216 | 1.00 | 12.07 | W |
| ATOM | 8251 | OH2 | WAT | W | 70 | 11.858 | 51.549 | 14.335 | 1.00 | 16.74 | W |
| ATOM | 8252 | OH2 | WAT | W | 72 | 17.143 | 56.270 | 10.273 | 1.00 | 13.60 | W |
| ATOM | 8253 | OH2 | WAT | W | 73 | 68.006 | 65.561 | −3.446 | 1.00 | 12.60 | W |
| ATOM | 8254 | OH2 | WAT | W | 75 | 22.889 | 53.007 | −24.420 | 1.00 | 14.72 | W |
| ATOM | 8255 | OH2 | WAT | W | 76 | 52.059 | 50.629 | 6.400 | 1.00 | 10.61 | W |
| ATOM | 8256 | OH2 | WAT | W | 77 | 17.622 | 52.779 | 6.356 | 1.00 | 11.97 | W |
| ATOM | 8257 | OH2 | WAT | W | 78 | 68.809 | 58.261 | −5.093 | 1.00 | 14.29 | W |
| ATOM | 8258 | OH2 | WAT | W | 79 | 33.495 | 48.113 | −10.195 | 1.00 | 8.82 | W |
| ATOM | 8259 | OH2 | WAT | W | 80 | 26.727 | 41.583 | −13.197 | 1.00 | 12.70 | W |
| ATOM | 8260 | OH2 | WAT | W | 81 | 26.797 | 60.896 | −3.250 | 1.00 | 10.29 | W |
| ATOM | 8261 | OH2 | WAT | W | 82 | 49.560 | 62.285 | −8.627 | 1.00 | 9.52 | W |
| ATOM | 8262 | OH2 | WAT | W | 83 | 41.032 | 52.406 | −16.918 | 1.00 | 12.51 | W |
| ATOM | 8263 | OH2 | WAT | W | 84 | 31.381 | 76.822 | −6.322 | 1.00 | 12.79 | W |
| ATOM | 8264 | OH2 | WAT | W | 85 | 43.389 | 68.256 | −24.999 | 1.00 | 14.18 | W |
| ATOM | 8265 | OH2 | WAT | W | 86 | 32.757 | 81.026 | −11.699 | 1.00 | 11.78 | W |
| ATOM | 8266 | OH2 | WAT | W | 87 | 20.527 | 66.154 | −19.601 | 1.00 | 12.21 | W |
| ATOM | 8267 | OH2 | WAT | W | 88 | 40.275 | 62.699 | 6.041 | 1.00 | 9.15 | W |
| ATOM | 8268 | OH2 | WAT | W | 89 | 42.187 | 47.294 | −24.509 | 1.00 | 14.89 | W |
| ATOM | 8269 | OH2 | WAT | W | 90 | 44.548 | 41.856 | −0.550 | 1.00 | 15.24 | W |
| ATOM | 8270 | OH2 | WAT | W | 91 | 38.578 | 55.195 | −31.685 | 1.00 | 14.89 | W |
| ATOM | 8271 | OH2 | WAT | W | 92 | 22.315 | 54.352 | −12.009 | 1.00 | 10.46 | W |
| ATOM | 8272 | OH2 | WAT | W | 93 | 48.241 | 89.207 | −30.694 | 1.00 | 15.15 | W |
| ATOM | 8273 | OH2 | WAT | W | 94 | 36.678 | 51.651 | 3.739 | 1.00 | 11.00 | W |
| ATOM | 8274 | OH2 | WAT | W | 95 | 38.700 | 62.268 | 8.378 | 1.00 | 12.20 | W |
| ATOM | 8275 | OH2 | WAT | W | 96 | 47.482 | 45.984 | 3.648 | 1.00 | 10.29 | W |
| ATOM | 8276 | OH2 | WAT | W | 97 | 33.607 | 57.028 | −35.919 | 1.00 | 18.29 | W |
| ATOM | 8277 | OH2 | WAT | W | 98 | 32.325 | 77.609 | −29.636 | 1.00 | 13.68 | W |
| ATOM | 8278 | OH2 | WAT | W | 99 | 39.188 | 38.924 | 17.246 | 1.00 | 16.44 | W |
| ATOM | 8279 | OH2 | WAT | W | 100 | 35.929 | 53.094 | 1.324 | 1.00 | 10.90 | W |
| ATOM | 8280 | OH2 | WAT | W | 101 | 14.480 | 60.026 | 5.140 | 1.00 | 14.27 | W |
| ATOM | 8281 | OH2 | WAT | W | 102 | 21.754 | 57.133 | 5.118 | 1.00 | 12.46 | W |
| ATOM | 8282 | OH2 | WAT | W | 103 | 43.069 | 48.058 | 11.256 | 1.00 | 12.39 | W |
| ATOM | 8283 | OH2 | WAT | W | 104 | 25.920 | 72.873 | 5.654 | 1.00 | 14.15 | W |
| ATOM | 8284 | OH2 | WAT | W | 105 | 27.565 | 48.488 | −21.578 | 1.00 | 14.53 | W |
| ATOM | 8285 | OH2 | WAT | W | 106 | 33.286 | 78.599 | −5.099 | 1.00 | 12.24 | W |
| ATOM | 8286 | OH2 | WAT | W | 107 | 50.058 | 59.512 | −11.520 | 1.00 | 10.57 | W |
| ATOM | 8287 | OH2 | WAT | W | 108 | 25.555 | 51.205 | −9.475 | 1.00 | 11.34 | W |
| ATOM | 8288 | OH2 | WAT | W | 109 | 40.153 | 56.225 | 5.779 | 1.00 | 12.76 | W |
| ATOM | 8289 | OH2 | WAT | W | 110 | 14.046 | 59.725 | −3.421 | 1.00 | 15.15 | W |
| ATOM | 8290 | OH2 | WAT | W | 111 | 19.679 | 53.575 | −11.765 | 1.00 | 11.58 | W |
| ATOM | 8291 | OH2 | WAT | W | 112 | 26.397 | 63.646 | 17.057 | 1.00 | 19.25 | W |
| ATOM | 8292 | OH2 | WAT | W | 113 | 19.842 | 57.363 | −11.722 | 1.00 | 15.86 | W |
| ATOM | 8293 | OH2 | WAT | W | 114 | 47.093 | 48.549 | 11.513 | 1.00 | 16.31 | W |
| ATOM | 8294 | OH2 | WAT | W | 115 | 67.773 | 81.656 | −25.681 | 1.00 | 12.31 | W |
| ATOM | 8295 | OH2 | WAT | W | 116 | 22.941 | 51.712 | −8.714 | 1.00 | 17.21 | W |
| ATOM | 8296 | OH2 | WAT | W | 117 | 46.531 | 70.323 | −36.707 | 1.00 | 15.24 | W |
| ATOM | 8297 | OH2 | WAT | W | 118 | 20.610 | 57.442 | −5.246 | 1.00 | 10.52 | W |
| ATOM | 8298 | OH2 | WAT | W | 119 | 18.199 | 60.639 | 24.362 | 1.00 | 19.10 | W |
| ATOM | 8299 | OH2 | WAT | W | 120 | 41.329 | 68.365 | −32.493 | 1.00 | 14.40 | W |
| ATOM | 8300 | OH2 | WAT | W | 121 | 38.335 | 40.047 | 1.670 | 1.00 | 13.35 | W |
| ATOM | 8301 | OH2 | WAT | W | 122 | 23.833 | 58.200 | 6.359 | 1.00 | 12.19 | W |
| ATOM | 8302 | OH2 | WAT | W | 123 | 18.240 | 59.718 | −20.036 | 1.00 | 17.38 | W |
| ATOM | 8303 | OH2 | WAT | W | 124 | 41.287 | 77.499 | −14.106 | 1.00 | 13.36 | W |
| ATOM | 8304 | OH2 | WAT | W | 125 | 52.832 | 58.921 | −1.733 | 1.00 | 12.54 | W |
| ATOM | 8305 | OH2 | WAT | W | 126 | 47.931 | 49.566 | 13.810 | 1.00 | 12.59 | W |
| ATOM | 8306 | OH2 | WAT | W | 127 | 35.204 | 43.627 | 28.724 | 1.00 | 14.94 | W |
| ATOM | 8307 | OH2 | WAT | W | 128 | 59.476 | 60.585 | −6.230 | 1.00 | 11.38 | W |
| ATOM | 8308 | OH2 | WAT | W | 129 | 53.626 | 72.514 | −19.200 | 1.00 | 12.06 | W |
| ATOM | 8309 | OH2 | WAT | W | 130 | 39.684 | 53.989 | −20.753 | 1.00 | 14.19 | W |
| ATOM | 8310 | OH2 | WAT | W | 131 | 33.156 | 36.626 | −4.367 | 1.00 | 14.10 | W |
| ATOM | 8311 | OH2 | WAT | W | 132 | 14.394 | 63.854 | −16.386 | 1.00 | 15.65 | W |
| ATOM | 8312 | OH2 | WAT | W | 133 | 47.541 | 42.987 | −2.387 | 1.00 | 16.31 | W |
| ATOM | 8313 | OH2 | WAT | W | 134 | 46.862 | 55.823 | −25.401 | 1.00 | 15.03 | W |
| ATOM | 8314 | OH2 | WAT | W | 136 | 32.224 | 58.564 | −28.544 | 1.00 | 13.45 | W |
| ATOM | 8315 | OH2 | WAT | W | 137 | 49.223 | 49.949 | 28.325 | 1.00 | 19.29 | W |
| ATOM | 8316 | OH2 | WAT | W | 138 | 26.323 | 37.326 | 29.416 | 1.00 | 19.14 | W |
| ATOM | 8317 | OH2 | WAT | W | 139 | 51.830 | 46.524 | 1.295 | 1.00 | 12.33 | W |
| ATOM | 8318 | OH2 | WAT | W | 140 | 41.682 | 58.681 | −16.050 | 1.00 | 12.26 | W |
| ATOM | 8319 | OH2 | WAT | W | 141 | 13.888 | 54.182 | 4.446 | 1.00 | 16.48 | W |
| ATOM | 8320 | OH2 | WAT | W | 142 | 67.770 | 78.875 | −25.268 | 1.00 | 12.57 | W |
| ATOM | 8321 | OH2 | WAT | W | 143 | 42.134 | 75.232 | −15.965 | 1.00 | 14.75 | W |
| ATOM | 8322 | OH2 | WAT | W | 144 | 23.116 | 55.996 | 30.863 | 1.00 | 16.59 | W |
| ATOM | 8323 | OH2 | WAT | W | 145 | 56.923 | 61.000 | 1.609 | 1.00 | 15.10 | W |
| ATOM | 8324 | OH2 | WAT | W | 146 | 20.034 | 74.341 | −6.831 | 1.00 | 14.78 | W |
| ATOM | 8325 | OH2 | WAT | W | 147 | 13.513 | 53.486 | 1.857 | 1.00 | 19.02 | W |
| ATOM | 8326 | OH2 | WAT | W | 148 | 34.238 | 34.360 | 16.148 | 1.00 | 20.31 | W |
| ATOM | 8327 | OH2 | WAT | W | 149 | 34.031 | 64.487 | −32.287 | 1.00 | 13.24 | W |
| ATOM | 8328 | OH2 | WAT | W | 150 | 14.014 | 49.063 | 15.378 | 1.00 | 17.37 | W |
| ATOM | 8329 | OH2 | WAT | W | 151 | 30.347 | 35.148 | −12.372 | 1.00 | 20.68 | W |
| ATOM | 8330 | OH2 | WAT | W | 152 | 18.389 | 54.932 | 14.146 | 1.00 | 14.59 | W |
| ATOM | 8331 | OH2 | WAT | W | 153 | 15.531 | 60.384 | 15.495 | 1.00 | 15.81 | W |
| ATOM | 8332 | OH2 | WAT | W | 154 | 16.835 | 74.574 | −2.964 | 1.00 | 16.76 | W |
| ATOM | 8333 | OH2 | WAT | W | 155 | 56.537 | 46.186 | −17.410 | 1.00 | 16.90 | W |
| ATOM | 8334 | OH2 | WAT | W | 156 | 45.674 | 59.945 | −18.274 | 1.00 | 12.93 | W |
| ATOM | 8335 | OH2 | WAT | W | 157 | 28.029 | 84.517 | −35.413 | 1.00 | 16.49 | W |
| ATOM | 8336 | OH2 | WAT | W | 158 | 73.011 | 65.045 | −7.742 | 1.00 | 18.99 | W |
| ATOM | 8337 | OH2 | WAT | W | 159 | 73.834 | 65.977 | −17.552 | 1.00 | 17.40 | W |
| ATOM | 8338 | OH2 | WAT | W | 160 | 59.358 | 49.999 | −0.640 | 1.00 | 16.59 | W |
| ATOM | 8339 | OH2 | WAT | W | 161 | 23.060 | 78.311 | −24.854 | 1.00 | 16.96 | W |
| ATOM | 8340 | OH2 | WAT | W | 162 | 14.343 | 54.351 | −16.113 | 1.00 | 16.49 | W |
| ATOM | 8341 | OH2 | WAT | W | 163 | 43.420 | 73.075 | 5.809 | 1.00 | 11.62 | W |
| ATOM | 8342 | OH2 | WAT | W | 164 | 11.844 | 55.012 | −12.346 | 1.00 | 15.84 | W |
| ATOM | 8343 | OH2 | WAT | W | 165 | 63.502 | 44.660 | −6.843 | 1.00 | 14.23 | W |
| ATOM | 8344 | OH2 | WAT | W | 166 | 24.666 | 51.102 | −24.012 | 1.00 | 13.46 | W |
| ATOM | 8345 | OH2 | WAT | W | 167 | 35.548 | 45.275 | −27.375 | 1.00 | 19.03 | W |
| ATOM | 8346 | OH2 | WAT | W | 168 | 28.659 | 49.712 | −24.380 | 1.00 | 17.39 | W |
| ATOM | 8347 | OH2 | WAT | W | 169 | 54.858 | 81.069 | −23.538 | 1.00 | 13.45 | W |
| ATOM | 8348 | OH2 | WAT | W | 170 | 24.206 | 63.494 | 7.574 | 1.00 | 12.75 | W |
| ATOM | 8349 | OH2 | WAT | W | 171 | 49.747 | 57.598 | −7.598 | 1.00 | 10.26 | W |
| ATOM | 8350 | OH2 | WAT | W | 172 | 45.989 | 70.940 | 0.068 | 1.00 | 14.02 | W |
| ATOM | 8351 | OH2 | WAT | W | 173 | 24.402 | 55.716 | −1.779 | 1.00 | 13.43 | W |
| ATOM | 8352 | OH2 | WAT | W | 174 | 28.620 | 41.291 | −21.645 | 1.00 | 14.74 | W |
| ATOM | 8353 | OH2 | WAT | W | 175 | 37.102 | 45.554 | 27.479 | 1.00 | 13.25 | W |
| ATOM | 8354 | OH2 | WAT | W | 176 | 41.571 | 41.193 | −17.611 | 1.00 | 18.25 | W |
| ATOM | 8355 | OH2 | WAT | W | 177 | 40.223 | 62.477 | −31.989 | 1.00 | 14.62 | W |
| ATOM | 8356 | OH2 | WAT | W | 178 | 49.238 | 59.310 | −28.693 | 1.00 | 20.38 | W |
| ATOM | 8357 | OH2 | WAT | W | 179 | 64.381 | 39.377 | −10.391 | 1.00 | 21.32 | W |
| ATOM | 8358 | OH2 | WAT | W | 180 | 19.168 | 72.461 | 3.018 | 1.00 | 18.78 | W |
| ATOM | 8359 | OH2 | WAT | W | 181 | 17.075 | 70.268 | −16.266 | 1.00 | 12.96 | W |
| ATOM | 8360 | OH2 | WAT | W | 182 | 9.392 | 52.493 | 16.921 | 1.00 | 23.15 | W |
| ATOM | 8361 | OH2 | WAT | W | 183 | 53.939 | 71.254 | −36.857 | 1.00 | 13.91 | W |
| ATOM | 8362 | OH2 | WAT | W | 184 | 50.885 | 70.915 | −23.550 | 1.00 | 16.66 | W |
| ATOM | 8363 | OH2 | WAT | W | 185 | 49.412 | 44.201 | 4.399 | 1.00 | 13.57 | W |
| ATOM | 8364 | OH2 | WAT | W | 186 | 59.279 | 68.219 | 2.499 | 1.00 | 19.23 | W |
| ATOM | 8365 | OH2 | WAT | W | 187 | 25.484 | 53.702 | 11.101 | 1.00 | 17.16 | W |
| ATOM | 8366 | OH2 | WAT | W | 188 | 69.101 | 61.416 | −2.408 | 1.00 | 16.03 | W |
| ATOM | 8367 | OH2 | WAT | W | 189 | 58.277 | 77.322 | −38.485 | 1.00 | 14.92 | W |
| ATOM | 8368 | OH2 | WAT | W | 190 | 44.347 | 46.042 | 9.977 | 1.00 | 14.65 | W |
| ATOM | 8369 | OH2 | WAT | W | 191 | 46.569 | 57.274 | −18.708 | 1.00 | 13.28 | W |
| ATOM | 8370 | OH2 | WAT | W | 192 | 33.581 | 83.552 | −10.326 | 1.00 | 15.89 | W |
| ATOM | 8371 | OH2 | WAT | W | 193 | 47.072 | 68.587 | −30.250 | 1.00 | 17.11 | W |
| ATOM | 8372 | OH2 | WAT | W | 194 | 44.472 | 56.046 | −15.367 | 1.00 | 16.02 | W |
| ATOM | 8373 | OH2 | WAT | W | 195 | 13.135 | 66.801 | −4.751 | 1.00 | 16.44 | W |
| ATOM | 8374 | OH2 | WAT | W | 197 | 24.508 | 39.081 | −11.494 | 1.00 | 20.79 | W |
| ATOM | 8375 | OH2 | WAT | W | 198 | 41.658 | 61.212 | −29.773 | 1.00 | 17.50 | W |
| ATOM | 8376 | OH2 | WAT | W | 199 | 27.188 | 41.368 | 13.298 | 1.00 | 15.13 | W |
| ATOM | 8377 | OH2 | WAT | W | 200 | 42.058 | 87.798 | −40.915 | 1.00 | 15.24 | W |
| ATOM | 8378 | OH2 | WAT | W | 201 | 70.003 | 69.894 | −12.626 | 1.00 | 14.74 | W |
| ATOM | 8379 | OH2 | WAT | W | 202 | 64.179 | 77.927 | −35.248 | 1.00 | 14.07 | W |
| ATOM | 8380 | OH2 | WAT | W | 203 | 20.804 | 65.342 | 7.755 | 1.00 | 17.03 | W |
| ATOM | 8381 | OH2 | WAT | W | 204 | 11.345 | 61.251 | −18.469 | 1.00 | 19.28 | W |
| ATOM | 8382 | OH2 | WAT | W | 205 | 24.586 | 70.556 | −8.728 | 1.00 | 13.10 | W |
| ATOM | 8383 | OH2 | WAT | W | 206 | 25.042 | 40.064 | 14.311 | 1.00 | 17.71 | W |
| ATOM | 8384 | OH2 | WAT | W | 207 | 22.658 | 63.257 | −33.219 | 1.00 | 18.83 | W |
| ATOM | 8385 | OH2 | WAT | W | 208 | 41.629 | 43.251 | 7.933 | 1.00 | 14.67 | W |
| ATOM | 8386 | OH2 | WAT | W | 209 | 20.931 | 51.833 | 47.478 | 1.00 | 16.92 | W |
| ATOM | 8387 | OH2 | WAT | W | 210 | 41.310 | 50.343 | 33.238 | 1.00 | 20.76 | W |
| ATOM | 8388 | OH2 | WAT | W | 211 | 45.951 | 52.717 | −19.108 | 1.00 | 16.03 | W |
| ATOM | 8389 | OH2 | WAT | W | 212 | 51.426 | 76.342 | −40.924 | 1.00 | 25.44 | W |
| ATOM | 8390 | OH2 | WAT | W | 213 | 25.622 | 86.174 | −31.896 | 1.00 | 16.09 | W |
| ATOM | 8391 | OH2 | WAT | W | 214 | 12.541 | 56.291 | 5.425 | 1.00 | 17.15 | W |
| ATOM | 8392 | OH2 | WAT | W | 215 | 22.523 | 56.813 | −34.105 | 1.00 | 17.30 | W |
| ATOM | 8393 | OH2 | WAT | W | 216 | 43.796 | 72.560 | −41.912 | 1.00 | 18.18 | W |
| ATOM | 8394 | OH2 | WAT | W | 217 | 44.637 | 58.659 | −15.909 | 1.00 | 16.17 | W |
| ATOM | 8395 | OH2 | WAT | W | 218 | 31.266 | 81.750 | 0.557 | 1.00 | 18.18 | W |
| ATOM | 8396 | OH2 | WAT | W | 219 | 52.052 | 56.397 | −30.316 | 1.00 | 20.40 | W |
| ATOM | 8397 | OH2 | WAT | W | 220 | 17.201 | 70.010 | −23.298 | 1.00 | 21.76 | W |
| ATOM | 8398 | OH2 | WAT | W | 221 | 37.700 | 46.928 | 29.873 | 1.00 | 18.65 | W |
| ATOM | 8399 | OH2 | WAT | W | 222 | 49.781 | 55.079 | −29.054 | 1.00 | 18.35 | W |
| ATOM | 8400 | OH2 | WAT | W | 223 | 36.227 | 79.122 | −4.990 | 1.00 | 18.76 | W |
| ATOM | 8401 | OH2 | WAT | W | 224 | 62.292 | 62.423 | 25.645 | 1.00 | 21.53 | W |
| ATOM | 8402 | OH2 | WAT | W | 225 | 11.410 | 53.122 | 6.820 | 1.00 | 16.14 | W |
| ATOM | 8403 | OH2 | WAT | W | 226 | 13.875 | 60.774 | −22.493 | 1.00 | 17.65 | W |
| ATOM | 8404 | OH2 | WAT | W | 227 | 50.785 | 61.911 | 26.371 | 1.00 | 20.81 | W |
| ATOM | 8405 | OH2 | WAT | W | 228 | 68.609 | 78.746 | −18.153 | 1.00 | 20.98 | W |
| ATOM | 8406 | OH2 | WAT | W | 229 | 84.248 | 68.254 | −17.382 | 1.00 | 16.45 | W |
| ATOM | 8407 | OH2 | WAT | W | 230 | 21.921 | 40.287 | −8.763 | 1.00 | 25.24 | W |
| ATOM | 8408 | OH2 | WAT | W | 231 | 52.792 | 69.689 | 18.337 | 1.00 | 23.67 | W |
| ATOM | 8409 | OH2 | WAT | W | 233 | 42.635 | 53.302 | 35.623 | 1.00 | 21.27 | W |
| ATOM | 8410 | OH2 | WAT | W | 234 | 70.643 | 52.132 | −12.652 | 1.00 | 19.68 | W |
| ATOM | 8411 | OH2 | WAT | W | 235 | 34.668 | 70.219 | 13.058 | 1.00 | 21.89 | W |
| ATOM | 8412 | OH2 | WAT | W | 236 | 27.265 | 46.404 | 13.634 | 1.00 | 16.31 | W |
| ATOM | 8413 | OH2 | WAT | W | 237 | 14.320 | 49.792 | 19.854 | 1.00 | 16.31 | W |
| ATOM | 8414 | OH2 | WAT | W | 238 | 49.691 | 73.425 | −21.732 | 1.00 | 21.90 | W |
| ATOM | 8415 | OH2 | WAT | W | 239 | 48.829 | 44.275 | −6.153 | 1.00 | 18.55 | W |
| ATOM | 8416 | OH2 | WAT | W | 240 | 61.398 | 76.794 | −39.730 | 1.00 | 19.88 | W |
| ATOM | 8417 | OH2 | WAT | W | 241 | 22.807 | 78.638 | −30.326 | 1.00 | 23.15 | W |
| ATOM | 8418 | OH2 | WAT | W | 242 | 43.596 | 88.461 | −38.551 | 1.00 | 15.45 | W |
| ATOM | 8419 | OH2 | WAT | W | 243 | 35.783 | 77.456 | −7.287 | 1.00 | 14.79 | W |
| ATOM | 8420 | OH2 | WAT | W | 244 | 24.815 | 68.296 | 21.838 | 1.00 | 19.19 | W |
| ATOM | 8421 | OH2 | WAT | W | 245 | 68.075 | 49.207 | −1.108 | 1.00 | 19.95 | W |
| ATOM | 8422 | OH2 | WAT | W | 246 | 54.280 | 47.750 | 15.537 | 1.00 | 20.36 | W |
| ATOM | 8423 | OH2 | WAT | W | 247 | 55.069 | 50.670 | 16.796 | 1.00 | 24.95 | W |
| ATOM | 8424 | OH2 | WAT | W | 248 | 39.430 | 48.768 | −32.780 | 1.00 | 23.67 | W |
| ATOM | 8425 | OH2 | WAT | W | 249 | 22.569 | 45.303 | −13.489 | 1.00 | 19.28 | W |
| ATOM | 8426 | OH2 | WAT | W | 250 | 67.180 | 55.272 | 0.972 | 1.00 | 14.46 | W |
| ATOM | 8427 | OH2 | WAT | W | 251 | 47.980 | 44.000 | −8.660 | 1.00 | 18.47 | W |
| ATOM | 8428 | OH2 | WAT | W | 252 | 26.921 | 87.791 | −23.599 | 1.00 | 22.16 | W |
| ATOM | 8429 | OH2 | WAT | W | 253 | 18.923 | 48.496 | 37.716 | 1.00 | 19.77 | W |
| ATOM | 8430 | OH2 | WAT | W | 254 | 83.559 | 67.682 | −22.775 | 1.00 | 25.26 | W |
| ATOM | 8431 | OH2 | WAT | W | 255 | 42.958 | 59.618 | −18.847 | 1.00 | 23.27 | W |
| ATOM | 8432 | OH2 | WAT | W | 256 | 47.414 | 79.105 | −40.375 | 1.00 | 19.23 | W |
| ATOM | 8433 | OH2 | WAT | W | 258 | 8.630 | 57.709 | −6.814 | 1.00 | 21.71 | W |
| ATOM | 8434 | OH2 | WAT | W | 259 | 54.925 | 90.238 | −23.447 | 1.00 | 24.47 | W |
| ATOM | 8435 | OH2 | WAT | W | 260 | 27.570 | 36.487 | −19.572 | 1.00 | 23.33 | W |
| ATOM | 8436 | OH2 | WAT | W | 261 | 72.138 | 58.735 | −11.226 | 1.00 | 25.73 | W |
| ATOM | 8437 | OH2 | WAT | W | 262 | 34.255 | 67.576 | 12.146 | 1.00 | 16.16 | W |
| ATOM | 8438 | OH2 | WAT | W | 263 | 11.762 | 59.422 | −4.906 | 1.00 | 19.04 | W |
| ATOM | 8439 | OH2 | WAT | W | 264 | 23.706 | 45.995 | −15.875 | 1.00 | 18.11 | W |
| ATOM | 8440 | OH2 | WAT | W | 265 | 50.139 | 70.863 | −17.624 | 1.00 | 21.18 | W |
| ATOM | 8441 | OH2 | WAT | W | 266 | 45.026 | 90.016 | −25.430 | 1.00 | 19.76 | W |
| ATOM | 8442 | OH2 | WAT | W | 267 | 16.075 | 72.079 | −18.963 | 1.00 | 21.64 | W |
| ATOM | 8443 | OH2 | WAT | W | 268 | 20.115 | 52.673 | −24.264 | 1.00 | 18.48 | W |
| ATOM | 8444 | OH2 | WAT | W | 269 | 59.537 | 76.497 | −8.952 | 1.00 | 18.23 | W |
| ATOM | 8445 | OH2 | WAT | W | 270 | 66.843 | 60.061 | −27.172 | 1.00 | 20.64 | W |
| ATOM | 8446 | OH2 | WAT | W | 271 | 38.606 | 51.471 | −29.882 | 1.00 | 24.46 | W |
| ATOM | 8447 | OH2 | WAT | W | 272 | 40.396 | 82.765 | −10.648 | 1.00 | 14.99 | W |
| ATOM | 8448 | OH2 | WAT | W | 273 | 42.125 | 48.704 | 31.281 | 1.00 | 17.97 | W |
| ATOM | 8449 | OH2 | WAT | W | 274 | 24.053 | 52.876 | −5.945 | 1.00 | 25.34 | W |
| ATOM | 8450 | OH2 | WAT | W | 275 | 53.271 | 47.132 | −25.039 | 1.00 | 18.91 | W |
| ATOM | 8451 | OH2 | WAT | W | 277 | 32.411 | 89.129 | −43.013 | 1.00 | 24.77 | W |
| ATOM | 8452 | OH2 | WAT | W | 278 | 36.026 | 36.057 | −4.541 | 1.00 | 19.29 | W |
| ATOM | 8453 | OH2 | WAT | W | 279 | 70.071 | 82.956 | −29.789 | 1.00 | 21.14 | W |
| ATOM | 8454 | OH2 | WAT | W | 280 | 49.683 | 82.456 | −43.824 | 1.00 | 19.21 | W |
| ATOM | 8455 | OH2 | WAT | W | 281 | 68.342 | 79.734 | −34.832 | 1.00 | 23.24 | W |
| ATOM | 8456 | OH2 | WAT | W | 282 | 29.226 | 46.817 | 37.090 | 1.00 | 20.39 | W |
| ATOM | 8457 | OH2 | WAT | W | 283 | 18.522 | 69.968 | 4.257 | 1.00 | 15.36 | W |
| ATOM | 8458 | OH2 | WAT | W | 284 | 14.166 | 68.276 | −7.752 | 1.00 | 18.68 | W |
| ATOM | 8459 | OH2 | WAT | W | 285 | 50.272 | 72.263 | 9.147 | 1.00 | 23.64 | W |
| ATOM | 8460 | OH2 | WAT | W | 286 | 39.675 | 75.625 | −40.325 | 1.00 | 19.11 | W |
| ATOM | 8461 | OH2 | WAT | W | 287 | 28.646 | 65.114 | 19.694 | 1.00 | 17.93 | W |
| ATOM | 8462 | OH2 | WAT | W | 288 | 11.705 | 58.754 | 7.964 | 1.00 | 21.41 | W |
| ATOM | 8463 | OH2 | WAT | W | 289 | 52.597 | 79.046 | −17.448 | 1.00 | 22.13 | W |
| ATOM | 8464 | OH2 | WAT | W | 290 | 18.473 | 64.964 | −26.958 | 1.00 | 19.14 | W |
| ATOM | 8465 | OH2 | WAT | W | 291 | 44.625 | 79.359 | −33.259 | 1.00 | 19.78 | W |
| ATOM | 8466 | OH2 | WAT | W | 292 | 36.100 | 80.964 | 6.342 | 1.00 | 17.11 | W |
| ATOM | 8467 | OH2 | WAT | W | 293 | 16.566 | 60.591 | −28.155 | 1.00 | 19.52 | W |
| ATOM | 8468 | OH2 | WAT | W | 294 | 29.722 | 87.215 | −17.892 | 1.00 | 28.58 | W |
| ATOM | 8469 | OH2 | WAT | W | 295 | 8.813 | 55.020 | −6.064 | 1.00 | 20.11 | W |
| ATOM | 8470 | OH2 | WAT | W | 296 | 46.636 | 61.696 | −25.945 | 1.00 | 20.16 | W |
| ATOM | 8471 | OH2 | WAT | W | 297 | 42.898 | 82.218 | −9.891 | 1.00 | 24.94 | W |
| ATOM | 8472 | OH2 | WAT | W | 298 | 10.335 | 50.645 | 18.833 | 1.00 | 25.27 | W |
| ATOM | 8473 | OH2 | WAT | W | 299 | 12.852 | 42.541 | 12.566 | 1.00 | 17.91 | W |
| ATOM | 8474 | OH2 | WAT | W | 300 | 35.895 | 45.124 | 30.854 | 1.00 | 20.00 | W |
| ATOM | 8475 | OH2 | WAT | W | 301 | 15.792 | 75.161 | −11.626 | 1.00 | 21.01 | W |
| ATOM | 8476 | OH2 | WAT | W | 302 | 25.222 | 46.928 | −20.840 | 1.00 | 23.20 | W |
| ATOM | 8477 | OH2 | WAT | W | 303 | 56.890 | 64.828 | 24.222 | 1.00 | 25.71 | W |
| ATOM | 8478 | OH2 | WAT | W | 304 | 38.567 | 94.125 | −41.997 | 1.00 | 22.28 | W |
| ATOM | 8479 | OH2 | WAT | W | 305 | 34.734 | 59.452 | −34.927 | 1.00 | 19.36 | W |
| ATOM | 8480 | OH2 | WAT | W | 306 | 33.032 | 41.250 | −17.616 | 1.00 | 25.42 | W |
| ATOM | 8481 | OH2 | WAT | W | 307 | 20.752 | 42.619 | −7.465 | 1.00 | 17.80 | W |
| ATOM | 8482 | OH2 | WAT | W | 308 | 19.062 | 76.610 | 4.854 | 1.00 | 27.92 | W |
| ATOM | 8483 | OH2 | WAT | W | 309 | 43.300 | 80.722 | 5.014 | 1.00 | 22.89 | W |
| ATOM | 8484 | OH2 | WAT | W | 310 | 32.873 | 85.309 | −5.565 | 1.00 | 20.83 | W |
| ATOM | 8485 | OH2 | WAT | W | 311 | 31.185 | 33.495 | 2.792 | 1.00 | 24.76 | W |
| ATOM | 8486 | OH2 | WAT | W | 312 | 21.436 | 78.463 | −1.341 | 1.00 | 20.14 | W |
| ATOM | 8487 | OH2 | WAT | W | 313 | 55.286 | 59.005 | 3.952 | 1.00 | 16.44 | W |
| ATOM | 8488 | OH2 | WAT | W | 314 | 13.406 | 62.292 | −19.877 | 1.00 | 20.13 | W |
| ATOM | 8489 | OH2 | WAT | W | 315 | 16.922 | 50.824 | −19.239 | 1.00 | 24.71 | W |
| ATOM | 8490 | OH2 | WAT | W | 316 | 14.323 | 62.818 | −12.022 | 1.00 | 20.05 | W |
| ATOM | 8491 | OH2 | WAT | W | 317 | 53.131 | 86.670 | −21.375 | 1.00 | 24.00 | W |
| ATOM | 8492 | OH2 | WAT | W | 318 | 70.984 | 49.511 | −0.110 | 1.00 | 23.19 | W |
| ATOM | 8493 | OH2 | WAT | W | 319 | 58.047 | 42.402 | 1.566 | 1.00 | 23.65 | W |
| ATOM | 8494 | OH2 | WAT | W | 320 | 74.768 | 76.369 | −14.340 | 1.00 | 27.79 | W |
| ATOM | 8495 | OH2 | WAT | W | 321 | 24.787 | 83.128 | −16.877 | 1.00 | 20.99 | W |
| ATOM | 8496 | OH2 | WAT | W | 322 | 56.915 | 67.700 | −35.396 | 1.00 | 21.88 | W |
| ATOM | 8497 | OH2 | WAT | W | 323 | 48.769 | 50.559 | −29.520 | 1.00 | 24.35 | W |
| ATOM | 8498 | OH2 | WAT | W | 325 | 66.709 | 71.607 | −6.852 | 1.00 | 19.70 | W |
| ATOM | 8499 | OH2 | WAT | W | 326 | 28.790 | 93.746 | −38.512 | 1.00 | 23.49 | W |
| ATOM | 8500 | OH2 | WAT | W | 327 | 32.509 | 64.768 | −9.926 | 1.00 | 17.49 | W |
| ATOM | 8501 | OH2 | WAT | W | 328 | 13.454 | 50.922 | −1.603 | 1.00 | 22.73 | W |
| ATOM | 8502 | OH2 | WAT | W | 329 | 39.346 | 49.244 | 30.155 | 1.00 | 19.73 | W |
| ATOM | 8503 | OH2 | WAT | W | 330 | 67.888 | 58.663 | −24.054 | 1.00 | 19.93 | W |
| ATOM | 8504 | OH2 | WAT | W | 331 | 20.779 | 58.985 | −34.524 | 1.00 | 18.17 | W |
| ATOM | 8505 | OH2 | WAT | W | 332 | 37.550 | 75.641 | −42.326 | 1.00 | 19.39 | W |
| ATOM | 8506 | OH2 | WAT | W | 333 | 31.895 | 51.294 | −35.392 | 1.00 | 20.49 | W |
| ATOM | 8507 | OH2 | WAT | W | 334 | 12.860 | 71.207 | −0.322 | 1.00 | 26.50 | W |
| ATOM | 8508 | OH2 | WAT | W | 335 | 37.017 | 41.136 | −24.664 | 1.00 | 26.87 | W |
| ATOM | 8509 | OH2 | WAT | W | 336 | 19.245 | 42.748 | −2.573 | 1.00 | 24.91 | W |
| ATOM | 8510 | OH2 | WAT | W | 337 | 50.102 | 44.093 | 7.091 | 1.00 | 17.71 | W |
| ATOM | 8511 | OH2 | WAT | W | 338 | 37.447 | 45.431 | −25.539 | 1.00 | 18.16 | W |
| ATOM | 8512 | OH2 | WAT | W | 339 | 75.300 | 74.056 | −19.617 | 1.00 | 26.79 | W |
| ATOM | 8513 | OH2 | WAT | W | 340 | 50.344 | 61.083 | −31.161 | 1.00 | 19.20 | W |
| ATOM | 8514 | OH2 | WAT | W | 341 | 42.877 | 56.717 | −31.559 | 1.00 | 24.68 | W |
| ATOM | 8515 | OH2 | WAT | W | 342 | 13.262 | 68.814 | 0.935 | 1.00 | 21.56 | W |
| ATOM | 8516 | OH2 | WAT | W | 343 | 40.735 | 82.850 | −31.321 | 1.00 | 26.31 | W |
| ATOM | 8517 | OH2 | WAT | W | 344 | 27.084 | 52.281 | 22.512 | 1.00 | 28.70 | W |
| ATOM | 8518 | OH2 | WAT | W | 345 | 12.534 | 66.379 | −9.439 | 1.00 | 20.41 | W |
| ATOM | 8519 | OH2 | WAT | W | 346 | 42.619 | 39.443 | −15.769 | 1.00 | 22.12 | W |
| ATOM | 8520 | OH2 | WAT | W | 347 | 48.091 | 48.434 | 16.393 | 1.00 | 17.55 | W |
| ATOM | 8521 | OH2 | WAT | W | 348 | 11.547 | 46.877 | 17.887 | 1.00 | 24.28 | W |
| ATOM | 8522 | OH2 | WAT | W | 349 | 16.474 | 67.738 | −19.969 | 1.00 | 19.28 | W |
| ATOM | 8523 | OH2 | WAT | W | 350 | 5.038 | 52.914 | −5.886 | 1.00 | 27.53 | W |
| ATOM | 8524 | OH2 | WAT | W | 352 | 56.273 | 90.997 | −25.706 | 1.00 | 22.05 | W |
| ATOM | 8525 | OH2 | WAT | W | 353 | 39.139 | 83.260 | −27.376 | 1.00 | 21.38 | W |
| ATOM | 8526 | OH2 | WAT | W | 354 | 60.719 | 56.850 | 15.845 | 1.00 | 26.22 | W |
| ATOM | 8527 | OH2 | WAT | W | 355 | 13.419 | 51.471 | 21.730 | 1.00 | 19.40 | W |
| ATOM | 8528 | OH2 | WAT | W | 356 | 43.394 | 93.839 | −37.655 | 1.00 | 21.87 | W |
| ATOM | 8529 | OH2 | WAT | W | 357 | 28.161 | 35.317 | −6.921 | 1.00 | 20.76 | W |
| ATOM | 8530 | OH2 | WAT | W | 358 | 80.258 | 64.057 | −21.537 | 1.00 | 25.16 | W |
| ATOM | 8531 | OH2 | WAT | W | 359 | 51.563 | 45.062 | 9.678 | 1.00 | 28.77 | W |
| ATOM | 8532 | OH2 | WAT | W | 360 | 21.446 | 46.840 | −17.598 | 1.00 | 23.85 | W |
| ATOM | 8533 | OH2 | WAT | W | 361 | 47.431 | 71.776 | −20.273 | 1.00 | 25.46 | W |
| ATOM | 8534 | OH2 | WAT | W | 362 | 21.320 | 85.819 | −31.086 | 1.00 | 28.86 | W |
| ATOM | 8535 | OH2 | WAT | W | 363 | 32.967 | 66.868 | 21.309 | 1.00 | 21.01 | W |
| ATOM | 8536 | OH2 | WAT | W | 364 | 14.670 | 61.989 | −14.512 | 1.00 | 20.46 | W |
| ATOM | 8537 | OH2 | WAT | W | 365 | 50.391 | 73.726 | −25.274 | 1.00 | 22.28 | W |
| ATOM | 8538 | OH2 | WAT | W | 366 | 41.946 | 79.336 | −23.444 | 1.00 | 25.26 | W |
| ATOM | 8539 | OH2 | WAT | W | 367 | 40.090 | 45.369 | 25.381 | 1.00 | 22.87 | W |
| ATOM | 8540 | OH2 | WAT | W | 368 | 50.764 | 76.382 | 14.494 | 1.00 | 20.54 | W |
| ATOM | 8541 | OH2 | WAT | W | 369 | 38.488 | 45.146 | −33.680 | 1.00 | 23.10 | W |
| ATOM | 8542 | OH2 | WAT | W | 370 | 61.315 | 54.313 | −28.914 | 1.00 | 24.07 | W |
| ATOM | 8543 | OH2 | WAT | W | 371 | 53.733 | 46.764 | 23.505 | 1.00 | 28.94 | W |
| ATOM | 8544 | OH2 | WAT | W | 372 | 56.967 | 43.140 | −20.141 | 1.00 | 24.44 | W |
| ATOM | 8545 | OH2 | WAT | W | 373 | 67.533 | 54.709 | −18.257 | 1.00 | 21.67 | W |
| ATOM | 8546 | OH2 | WAT | W | 374 | 42.392 | 79.852 | −31.723 | 1.00 | 29.17 | W |
| ATOM | 8547 | OH2 | WAT | W | 375 | 29.171 | 62.237 | −39.774 | 1.00 | 22.24 | W |
| ATOM | 8548 | OH2 | WAT | W | 376 | 27.168 | 61.164 | 10.332 | 1.00 | 24.42 | W |
| ATOM | 8549 | OH2 | WAT | W | 377 | 45.832 | 45.100 | 23.009 | 1.00 | 26.77 | W |
| ATOM | 8550 | OH2 | WAT | W | 378 | 34.919 | 51.178 | 35.930 | 1.00 | 27.88 | W |
| ATOM | 8551 | OH2 | WAT | W | 379 | 57.740 | 62.410 | 7.853 | 1.00 | 25.59 | W |
| ATOM | 8552 | OH2 | WAT | W | 380 | 25.278 | 33.367 | 13.289 | 1.00 | 24.89 | W |
| ATOM | 8553 | OH2 | WAT | W | 381 | 27.478 | 59.295 | 8.589 | 1.00 | 20.20 | W |
| ATOM | 8554 | OH2 | WAT | W | 382 | 42.720 | 94.359 | −30.466 | 1.00 | 30.57 | W |
| ATOM | 8555 | OH2 | WAT | W | 383 | 13.949 | 58.005 | −1.367 | 1.00 | 19.27 | W |
| ATOM | 8556 | OH2 | WAT | W | 384 | 49.207 | 76.417 | 5.694 | 1.00 | 28.48 | W |
| ATOM | 8557 | OH2 | WAT | W | 385 | 27.814 | 67.606 | 28.196 | 1.00 | 26.23 | W |
| ATOM | 8558 | OH2 | WAT | W | 386 | 41.984 | 39.094 | 16.733 | 1.00 | 24.84 | W |
| ATOM | 8559 | OH2 | WAT | W | 387 | 58.873 | 46.425 | −1.374 | 1.00 | 21.53 | W |
| ATOM | 8560 | OH2 | WAT | W | 388 | 73.274 | 76.732 | −18.311 | 1.00 | 26.55 | W |
| ATOM | 8561 | OH2 | WAT | W | 389 | 19.687 | 39.332 | 17.591 | 1.00 | 18.94 | W |
| ATOM | 8562 | OH2 | WAT | W | 390 | 39.662 | 58.933 | −18.087 | 1.00 | 19.74 | W |
| ATOM | 8563 | OH2 | WAT | W | 391 | 28.147 | 47.005 | −31.558 | 1.00 | 25.38 | W |
| ATOM | 8564 | OH2 | WAT | W | 392 | 68.586 | 46.914 | −16.614 | 1.00 | 27.92 | W |
| ATOM | 8565 | OH2 | WAT | W | 393 | 66.468 | 62.581 | −13.049 | 1.00 | 17.12 | W |
| ATOM | 8566 | OH2 | WAT | W | 394 | 26.521 | 75.061 | 2.145 | 1.00 | 22.89 | W |
| ATOM | 8567 | OH2 | WAT | W | 395 | 40.061 | 39.148 | 9.051 | 1.00 | 24.66 | W |
| ATOM | 8568 | OH2 | WAT | W | 396 | 21.299 | 47.235 | 39.151 | 1.00 | 23.69 | W |
| ATOM | 8569 | OH2 | WAT | W | 397 | 42.155 | 74.782 | −41.480 | 1.00 | 25.00 | W |
| ATOM | 8570 | OH2 | WAT | W | 398 | 14.628 | 55.293 | 27.232 | 1.00 | 32.51 | W |
| ATOM | 8571 | OH2 | WAT | W | 399 | 28.625 | 59.754 | −38.993 | 1.00 | 26.37 | W |
| ATOM | 8572 | OH2 | WAT | W | 400 | 38.975 | 42.908 | 22.645 | 1.00 | 25.45 | W |
| ATOM | 8573 | OH2 | WAT | W | 401 | 16.227 | 52.945 | −22.427 | 1.00 | 28.89 | W |
| ATOM | 8574 | OH2 | WAT | W | 402 | 27.322 | 34.952 | 29.323 | 1.00 | 24.65 | W |
| ATOM | 8575 | OH2 | WAT | W | 403 | 17.137 | 80.720 | −7.430 | 1.00 | 25.21 | W |
| ATOM | 8576 | OH2 | WAT | W | 404 | 48.222 | 41.802 | 3.682 | 1.00 | 24.08 | W |
| ATOM | 8577 | OH2 | WAT | W | 405 | 73.340 | 51.338 | −11.365 | 1.00 | 28.24 | W |
| ATOM | 8578 | OH2 | WAT | W | 406 | 58.671 | 53.682 | −22.208 | 1.00 | 22.00 | W |
| ATOM | 8579 | OH2 | WAT | W | 407 | 47.526 | 79.533 | −18.523 | 1.00 | 22.94 | W |
| ATOM | 8580 | OH2 | WAT | W | 408 | 40.691 | 96.492 | −37.372 | 1.00 | 21.41 | W |
| ATOM | 8581 | OH2 | WAT | W | 409 | 60.926 | 63.677 | 15.141 | 1.00 | 27.39 | W |
| ATOM | 8582 | OH2 | WAT | W | 410 | 59.335 | 37.649 | −15.222 | 1.00 | 26.73 | W |
| ATOM | 8583 | OH2 | WAT | W | 411 | 20.923 | 30.305 | 17.732 | 1.00 | 29.75 | W |
| ATOM | 8584 | OH2 | WAT | W | 412 | 46.041 | 96.943 | −43.820 | 1.00 | 21.70 | W |
| ATOM | 8585 | OH2 | WAT | W | 413 | 21.543 | 86.099 | −19.119 | 1.00 | 29.10 | W |
| ATOM | 8586 | OH2 | WAT | W | 414 | 20.561 | 68.418 | 22.243 | 1.00 | 25.39 | W |
| ATOM | 8587 | OH2 | WAT | W | 415 | 45.142 | 69.283 | −17.334 | 1.00 | 24.13 | W |
| ATOM | 8588 | OH2 | WAT | W | 416 | 17.330 | 70.661 | 6.541 | 1.00 | 31.13 | W |
| ATOM | 8589 | OH2 | WAT | W | 417 | 51.111 | 52.780 | −28.613 | 1.00 | 21.15 | W |
| ATOM | 8590 | OH2 | WAT | W | 418 | 19.096 | 83.349 | −25.745 | 1.00 | 29.17 | W |
| ATOM | 8591 | OH2 | WAT | W | 419 | 39.889 | 86.020 | −21.853 | 1.00 | 22.69 | W |
| ATOM | 8592 | OH2 | WAT | W | 420 | 22.570 | 66.011 | 29.297 | 1.00 | 28.90 | W |
| ATOM | 8593 | OH2 | WAT | W | 421 | 24.823 | 59.430 | −36.234 | 1.00 | 28.15 | W |
| ATOM | 8594 | OH2 | WAT | W | 422 | 58.331 | 34.694 | −6.071 | 1.00 | 28.92 | W |
| ATOM | 8595 | OH2 | WAT | W | 423 | 50.546 | 79.034 | −15.583 | 1.00 | 27.80 | W |
| ATOM | 8596 | OH2 | WAT | W | 424 | 17.745 | 68.564 | 24.329 | 1.00 | 32.50 | W |
| ATOM | 8597 | OH2 | WAT | W | 425 | 56.367 | 92.787 | −29.437 | 1.00 | 24.61 | W |
| ATOM | 8598 | OH2 | WAT | W | 426 | 52.556 | 73.131 | 4.237 | 1.00 | 33.92 | W |
| ATOM | 8599 | OH2 | WAT | W | 427 | 28.063 | 67.208 | 7.740 | 1.00 | 24.95 | W |
| ATOM | 8600 | OH2 | WAT | W | 428 | 22.030 | 36.775 | 0.168 | 1.00 | 21.71 | W |
| ATOM | 8601 | OH2 | WAT | W | 429 | 40.079 | 79.456 | −28.245 | 1.00 | 25.42 | W |
| ATOM | 8602 | OH2 | WAT | W | 430 | 10.576 | 77.090 | −5.015 | 1.00 | 30.97 | W |
| ATOM | 8603 | OH2 | WAT | W | 431 | 27.152 | 65.194 | 9.060 | 1.00 | 25.26 | W |
| ATOM | 8604 | OH2 | WAT | W | 432 | 72.751 | 64.037 | −20.984 | 1.00 | 26.96 | W |
| ATOM | 8605 | OH2 | WAT | W | 433 | 79.826 | 73.903 | −17.648 | 1.00 | 28.48 | W |
| ATOM | 8606 | OH2 | WAT | W | 434 | 66.841 | 74.355 | −4.271 | 1.00 | 30.30 | W |
| ATOM | 8607 | OH2 | WAT | W | 435 | 48.154 | 64.323 | −28.370 | 1.00 | 22.20 | W |
| ATOM | 8608 | OH2 | WAT | W | 436 | 42.519 | 39.358 | −1.503 | 1.00 | 25.77 | W |
| ATOM | 8609 | OH2 | WAT | W | 437 | 63.183 | 67.822 | −31.749 | 1.00 | 27.22 | W |
| ATOM | 8610 | OH2 | WAT | W | 438 | 24.165 | 41.927 | −14.685 | 1.00 | 23.45 | W |
| ATOM | 8611 | OH2 | WAT | W | 439 | 27.758 | 50.897 | 13.213 | 1.00 | 19.47 | W |
| ATOM | 8612 | OH2 | WAT | W | 440 | 38.638 | 83.552 | −39.787 | 1.00 | 22.96 | W |
| ATOM | 8613 | OH2 | WAT | W | 441 | 76.697 | 74.036 | −26.770 | 1.00 | 29.60 | W |
| ATOM | 8614 | OH2 | WAT | W | 442 | 28.767 | 72.466 | 22.467 | 1.00 | 25.85 | W |
| ATOM | 8615 | OH2 | WAT | W | 443 | 20.751 | 55.008 | −35.607 | 1.00 | 31.09 | W |
| ATOM | 8616 | OH2 | WAT | W | 444 | 24.098 | 57.242 | 38.823 | 1.00 | 24.73 | W |
| ATOM | 8617 | OH2 | WAT | W | 445 | 38.190 | 49.342 | −10.134 | 1.00 | 38.23 | W |
| ATOM | 8618 | OH2 | WAT | W | 447 | 23.326 | 82.586 | −40.633 | 1.00 | 24.86 | W |
| ATOM | 8619 | OH2 | WAT | W | 448 | 17.246 | 67.223 | −26.245 | 1.00 | 27.75 | W |
| ATOM | 8620 | OH2 | WAT | W | 449 | 66.919 | 84.334 | −22.218 | 1.00 | 23.81 | W |
| ATOM | 8621 | OH2 | WAT | W | 450 | 52.681 | 64.880 | −27.991 | 1.00 | 41.60 | W |
| ATOM | 8622 | OH2 | WAT | W | 451 | 20.704 | 68.074 | 8.597 | 1.00 | 21.60 | W |
| ATOM | 8623 | OH2 | WAT | W | 452 | 61.634 | 63.846 | −30.745 | 1.00 | 28.03 | W |
| ATOM | 8624 | OH2 | WAT | W | 453 | 9.806 | 59.916 | 6.504 | 1.00 | 29.44 | W |
| ATOM | 8625 | OH2 | WAT | W | 454 | 31.630 | 85.850 | −19.824 | 1.00 | 22.22 | W |
| ATOM | 8626 | OH2 | WAT | W | 455 | 28.063 | 88.041 | −41.579 | 1.00 | 27.55 | W |
| ATOM | 8627 | OH2 | WAT | W | 456 | 44.698 | 82.605 | −30.429 | 1.00 | 20.68 | W |
| ATOM | 8628 | OH2 | WAT | W | 457 | 59.485 | 67.175 | −34.677 | 1.00 | 26.13 | W |
| ATOM | 8629 | OH2 | WAT | W | 458 | 70.244 | 56.497 | −12.495 | 1.00 | 28.68 | W |
| ATOM | 8630 | OH2 | WAT | W | 459 | 46.088 | 46.117 | 16.230 | 1.00 | 29.47 | W |
| ATOM | 8631 | OH2 | WAT | W | 460 | 46.004 | 82.700 | −25.325 | 1.00 | 29.72 | W |
| ATOM | 8632 | OH2 | WAT | W | 461 | 56.907 | 87.672 | −20.060 | 1.00 | 25.77 | W |
| ATOM | 8633 | OH2 | WAT | W | 462 | 73.660 | 74.098 | −5.829 | 1.00 | 23.60 | W |
| ATOM | 8634 | OH2 | WAT | W | 463 | 19.646 | 43.105 | 33.835 | 1.00 | 25.04 | W |
| ATOM | 8635 | OH2 | WAT | W | 464 | 79.264 | 51.116 | −0.590 | 1.00 | 21.30 | W |
| ATOM | 8636 | OH2 | WAT | W | 465 | 66.047 | 45.418 | −3.282 | 1.00 | 26.67 | W |
| ATOM | 8637 | OH2 | WAT | W | 467 | 48.396 | 78.020 | −9.778 | 1.00 | 24.13 | W |
| ATOM | 8638 | OH2 | WAT | W | 468 | 29.998 | 34.084 | −1.514 | 1.00 | 22.90 | W |
| ATOM | 8639 | OH2 | WAT | W | 469 | 16.176 | 74.990 | −24.051 | 1.00 | 31.88 | W |
| ATOM | 8640 | OH2 | WAT | W | 470 | 17.617 | 39.367 | 35.721 | 1.00 | 38.09 | W |
| ATOM | 8641 | OH2 | WAT | W | 471 | 22.534 | 31.157 | 29.484 | 1.00 | 28.54 | W |
| ATOM | 8642 | OH2 | WAT | W | 472 | 33.369 | 84.313 | −18.330 | 1.00 | 15.97 | W |
| ATOM | 8643 | OH2 | WAT | W | 473 | 41.250 | 94.879 | −39.511 | 1.00 | 24.39 | W |
| ATOM | 8644 | OH2 | WAT | W | 474 | 44.232 | 83.630 | −27.639 | 1.00 | 23.37 | W |
| ATOM | 8645 | OH2 | WAT | W | 475 | 30.862 | 57.188 | 4.543 | 1.00 | 17.90 | W |
| ATOM | 8646 | OH2 | WAT | W | 476 | 65.908 | 66.089 | −0.376 | 1.00 | 24.05 | W |
| ATOM | 8647 | OH2 | WAT | W | 477 | 46.470 | 68.518 | 26.475 | 1.00 | 28.39 | W |
| ATOM | 8648 | OH2 | WAT | W | 478 | 70.032 | 61.711 | −20.342 | 1.00 | 28.95 | W |
| ATOM | 8649 | OH2 | WAT | W | 479 | 23.576 | 90.884 | −26.085 | 1.00 | 32.69 | W |
| ATOM | 8650 | OH2 | WAT | W | 480 | 25.257 | 71.144 | 22.415 | 1.00 | 28.48 | W |
| ATOM | 8651 | OH2 | WAT | W | 481 | 14.011 | 69.331 | 26.764 | 1.00 | 25.72 | W |
| ATOM | 8652 | OH2 | WAT | W | 482 | 62.091 | 80.708 | −18.944 | 1.00 | 22.51 | W |
| ATOM | 8653 | OH2 | WAT | W | 483 | 27.568 | 47.649 | 45.829 | 1.00 | 30.99 | W |
| ATOM | 8654 | OH2 | WAT | W | 484 | 24.924 | 34.183 | 0.541 | 1.00 | 31.69 | W |
| ATOM | 8655 | OH2 | WAT | W | 485 | 57.542 | 69.040 | 13.372 | 1.00 | 35.06 | W |
| ATOM | 8656 | OH2 | WAT | W | 486 | 7.964 | 48.092 | 11.826 | 1.00 | 24.78 | W |
| ATOM | 8657 | OH2 | WAT | W | 487 | 71.310 | 59.790 | 1.011 | 1.00 | 30.00 | W |
| ATOM | 8658 | OH2 | WAT | W | 488 | 67.619 | 88.039 | −23.404 | 1.00 | 24.62 | W |
| ATOM | 8659 | OH2 | WAT | W | 489 | 12.380 | 75.159 | −13.498 | 1.00 | 29.76 | W |
| ATOM | 8660 | OH2 | WAT | W | 490 | 27.878 | 52.141 | 42.550 | 1.00 | 28.52 | W |
| ATOM | 8661 | OH2 | WAT | W | 491 | 22.024 | 62.686 | 33.804 | 1.00 | 29.60 | W |
| ATOM | 8662 | OH2 | WAT | W | 492 | 59.396 | 66.922 | −26.287 | 1.00 | 39.41 | W |
| ATOM | 8663 | OH2 | WAT | W | 493 | 34.320 | 87.124 | −10.560 | 1.00 | 30.89 | W |
| ATOM | 8664 | OH2 | WAT | W | 494 | 30.216 | 36.445 | 29.080 | 1.00 | 25.09 | W |
| ATOM | 8665 | OH2 | WAT | W | 495 | 8.571 | 49.568 | 5.657 | 1.00 | 35.32 | W |
| ATOM | 8666 | OH2 | WAT | W | 498 | 41.955 | 67.344 | 29.326 | 1.00 | 25.76 | W |
| ATOM | 8667 | OH2 | WAT | W | 499 | 72.402 | 75.774 | −27.267 | 1.00 | 28.93 | W |
| ATOM | 8668 | OH2 | WAT | W | 500 | 8.848 | 44.348 | 12.960 | 1.00 | 29.54 | W |
| ATOM | 8669 | OH2 | WAT | W | 501 | 71.065 | 43.044 | −15.336 | 1.00 | 30.91 | W |
| ATOM | 8670 | OH2 | WAT | W | 502 | 41.462 | 44.667 | −24.401 | 1.00 | 26.41 | W |
| ATOM | 8671 | OH2 | WAT | W | 503 | 39.798 | 66.983 | 31.423 | 1.00 | 27.70 | W |
| ATOM | 8672 | OH2 | WAT | W | 504 | 53.321 | 52.719 | 26.356 | 1.00 | 25.71 | W |
| ATOM | 8673 | OH2 | WAT | W | 505 | 26.304 | 44.377 | −27.111 | 1.00 | 28.70 | W |
| ATOM | 8674 | OH2 | WAT | W | 506 | 19.649 | 52.518 | −27.061 | 1.00 | 26.07 | W |
| ATOM | 8675 | OH2 | WAT | W | 507 | 70.823 | 95.128 | −34.949 | 1.00 | 33.07 | W |
| ATOM | 8676 | OH2 | WAT | W | 508 | 48.072 | 64.729 | −25.801 | 1.00 | 25.60 | W |
| ATOM | 8677 | OH2 | WAT | W | 509 | 44.452 | 64.662 | −35.219 | 1.00 | 35.32 | W |
| ATOM | 8678 | OH2 | WAT | W | 510 | 18.126 | 52.856 | 35.976 | 1.00 | 29.35 | W |
| ATOM | 8679 | OH2 | WAT | W | 511 | 83.591 | 70.051 | −20.249 | 1.00 | 24.79 | W |
| ATOM | 8680 | OH2 | WAT | W | 512 | 43.836 | 79.945 | −42.480 | 1.00 | 26.76 | W |
| ATOM | 8681 | OH2 | WAT | W | 513 | 22.688 | 62.534 | 16.084 | 1.00 | 24.30 | W |
| ATOM | 8682 | OH2 | WAT | W | 514 | 45.760 | 79.249 | −7.906 | 1.00 | 24.96 | W |
| ATOM | 8683 | OH2 | WAT | W | 515 | 26.801 | 88.079 | −4.103 | 1.00 | 32.00 | W |
| ATOM | 8684 | OH2 | WAT | W | 516 | 38.743 | 42.182 | −22.199 | 1.00 | 30.07 | W |
| ATOM | 8685 | OH2 | WAT | W | 517 | 36.884 | 29.145 | 13.084 | 1.00 | 31.77 | W |
| ATOM | 8686 | OH2 | WAT | W | 518 | 20.410 | 65.507 | 27.349 | 1.00 | 30.30 | W |
| ATOM | 8687 | OH2 | WAT | W | 519 | 6.729 | 59.284 | 2.971 | 1.00 | 32.47 | W |
| ATOM | 8688 | OH2 | WAT | W | 520 | 61.217 | 77.418 | −12.932 | 1.00 | 22.54 | W |
| ATOM | 8689 | OH2 | WAT | W | 521 | 28.095 | 49.290 | 38.335 | 1.00 | 25.33 | W |
| ATOM | 8690 | OH2 | WAT | W | 522 | 29.442 | 41.131 | −25.576 | 1.00 | 24.39 | W |
| ATOM | 8691 | OH2 | WAT | W | 523 | 19.537 | 67.313 | −2.706 | 1.00 | 23.69 | W |
| ATOM | 8692 | OH2 | WAT | W | 524 | 43.446 | 41.720 | −7.635 | 1.00 | 26.49 | W |
| ATOM | 8693 | OH2 | WAT | W | 525 | 48.643 | 55.026 | −22.750 | 1.00 | 19.36 | W |
| ATOM | 8694 | OH2 | WAT | W | 526 | 26.385 | 81.719 | −3.635 | 1.00 | 41.05 | W |
| ATOM | 8695 | OH2 | WAT | W | 527 | 35.767 | 39.049 | −16.111 | 1.00 | 28.81 | W |
| ATOM | 8696 | OH2 | WAT | W | 528 | 58.571 | 51.181 | 22.159 | 1.00 | 21.40 | W |
| ATOM | 8697 | OH2 | WAT | W | 529 | 54.235 | 45.937 | 4.993 | 1.00 | 33.49 | W |
| ATOM | 8698 | OH2 | WAT | W | 530 | 46.524 | 80.242 | −42.585 | 1.00 | 25.30 | W |
| ATOM | 8699 | OH2 | WAT | W | 531 | 71.643 | 84.509 | −20.062 | 1.00 | 25.12 | W |
| ATOM | 8700 | OH2 | WAT | W | 532 | 61.128 | 67.499 | −36.724 | 1.00 | 28.07 | W |
| ATOM | 8701 | OH2 | WAT | W | 533 | 37.969 | 31.489 | 16.453 | 1.00 | 39.22 | W |
| ATOM | 8702 | OH2 | WAT | W | 534 | 58.573 | 34.864 | −12.260 | 1.00 | 28.74 | W |
| ATOM | 8703 | OH2 | WAT | W | 535 | 21.002 | 79.421 | −23.639 | 1.00 | 27.60 | W |
| ATOM | 8704 | OH2 | WAT | W | 536 | 51.295 | 71.304 | −20.170 | 1.00 | 17.32 | W |
| ATOM | 8705 | OH2 | WAT | W | 537 | 32.081 | 85.225 | −8.550 | 1.00 | 25.94 | W |
| ATOM | 8706 | OH2 | WAT | W | 538 | 21.710 | 29.081 | 28.041 | 1.00 | 31.53 | W |
| ATOM | 8707 | OH2 | WAT | W | 539 | 22.095 | 87.545 | −7.194 | 1.00 | 35.62 | W |
| ATOM | 8708 | OH2 | WAT | W | 540 | 31.578 | 57.434 | 37.279 | 1.00 | 27.30 | W |
| ATOM | 8709 | OH2 | WAT | W | 541 | 17.488 | 81.399 | −17.281 | 1.00 | 34.73 | W |
| ATOM | 8710 | OH2 | WAT | W | 542 | 51.884 | 58.999 | 6.332 | 1.00 | 41.97 | W |
| ATOM | 8711 | OH2 | WAT | W | 544 | 21.957 | 80.184 | −35.949 | 1.00 | 24.09 | W |
| ATOM | 8712 | OH2 | WAT | W | 545 | 24.829 | 74.038 | 9.017 | 1.00 | 26.59 | W |
| ATOM | 8713 | OH2 | WAT | W | 546 | 21.438 | 79.893 | 5.316 | 1.00 | 27.77 | W |
| ATOM | 8714 | OH2 | WAT | W | 547 | 48.301 | 94.016 | −37.157 | 1.00 | 32.94 | W |
| ATOM | 8715 | OH2 | WAT | W | 548 | 39.887 | 38.849 | −3.487 | 1.00 | 22.74 | W |
| ATOM | 8716 | OH2 | WAT | W | 549 | 39.630 | 96.139 | −28.202 | 1.00 | 36.55 | W |
| ATOM | 8717 | OH2 | WAT | W | 550 | 36.804 | 34.917 | −15.757 | 1.00 | 29.05 | W |
| ATOM | 8718 | OH2 | WAT | W | 551 | 34.920 | 45.669 | 33.370 | 1.00 | 19.31 | W |
| ATOM | 8719 | OH2 | WAT | W | 552 | 55.621 | 77.953 | −1.577 | 1.00 | 32.69 | W |
| ATOM | 8720 | OH2 | WAT | W | 553 | 70.242 | 61.953 | −0.089 | 1.00 | 28.70 | W |
| ATOM | 8721 | OH2 | WAT | W | 554 | 46.112 | 74.216 | 22.600 | 1.00 | 30.69 | W |
| ATOM | 8722 | OH2 | WAT | W | 555 | 48.424 | 46.019 | −23.172 | 1.00 | 27.05 | W |
| ATOM | 8723 | OH2 | WAT | W | 556 | 60.520 | 91.845 | −28.475 | 1.00 | 29.32 | W |
| ATOM | 8724 | OH2 | WAT | W | 557 | 37.929 | 62.300 | 35.419 | 1.00 | 28.02 | W |
| ATOM | 8725 | OH2 | WAT | W | 559 | 29.431 | 63.026 | −37.425 | 1.00 | 39.57 | W |
| ATOM | 8726 | OH2 | WAT | W | 560 | 61.433 | 48.378 | −31.702 | 1.00 | 37.37 | W |
| ATOM | 8727 | OH2 | WAT | W | 561 | 62.085 | 83.114 | −38.287 | 1.00 | 25.83 | W |
| ATOM | 8728 | OH2 | WAT | W | 562 | 62.988 | 42.869 | −20.951 | 1.00 | 25.61 | W |
| ATOM | 8729 | OH2 | WAT | W | 563 | 53.472 | 44.227 | 22.714 | 1.00 | 28.63 | W |
| ATOM | 8730 | OH2 | WAT | W | 564 | 30.645 | 34.628 | −9.707 | 1.00 | 30.01 | W |
| ATOM | 8731 | OH2 | WAT | W | 565 | 29.635 | 31.443 | 21.942 | 1.00 | 30.77 | W |
| ATOM | 8732 | OH2 | WAT | W | 566 | 78.625 | 69.011 | −12.005 | 1.00 | 25.23 | W |
| ATOM | 8733 | OH2 | WAT | W | 567 | 31.369 | 69.210 | −45.418 | 1.00 | 31.91 | W |
| ATOM | 8734 | OH2 | WAT | W | 568 | 73.477 | 82.280 | −21.188 | 1.00 | 26.33 | W |
| ATOM | 8735 | OH2 | WAT | W | 569 | 47.244 | 71.014 | −23.950 | 1.00 | 29.92 | W |
| ATOM | 8736 | OH2 | WAT | W | 570 | 20.446 | 37.879 | 12.137 | 1.00 | 27.15 | W |
| ATOM | 8737 | OH2 | WAT | W | 571 | 34.990 | 94.883 | −32.269 | 1.00 | 28.71 | W |
| ATOM | 8738 | OH2 | WAT | W | 572 | 17.230 | 41.552 | 9.181 | 1.00 | 29.09 | W |
| ATOM | 8739 | OH2 | WAT | W | 573 | 20.459 | 80.167 | −33.664 | 1.00 | 36.30 | W |
| ATOM | 8740 | OH2 | WAT | W | 574 | 49.151 | 91.694 | −24.092 | 1.00 | 27.95 | W |
| ATOM | 8741 | OH2 | WAT | W | 575 | 43.768 | 44.908 | 19.223 | 1.00 | 30.88 | W |
| ATOM | 8742 | OH2 | WAT | W | 576 | 21.182 | 35.533 | −5.151 | 1.00 | 30.52 | W |
| ATOM | 8743 | OH2 | WAT | W | 577 | 65.093 | 87.994 | −24.138 | 1.00 | 28.32 | W |
| ATOM | 8744 | OH2 | WAT | W | 578 | 46.841 | 53.785 | −21.480 | 1.00 | 24.30 | W |
| ATOM | 8745 | OH2 | WAT | W | 579 | 71.566 | 63.009 | −27.060 | 1.00 | 31.49 | W |
| ATOM | 8746 | OH2 | WAT | W | 580 | 19.970 | 35.544 | 25.719 | 1.00 | 26.74 | W |
| ATOM | 8747 | OH2 | WAT | W | 581 | 59.366 | 86.847 | −45.758 | 1.00 | 30.58 | W |
| ATOM | 8748 | OH2 | WAT | W | 582 | 51.734 | 34.233 | −7.865 | 1.00 | 37.38 | W |
| ATOM | 8749 | OH2 | WAT | W | 583 | 56.216 | 78.991 | −14.684 | 1.00 | 33.27 | W |
| ATOM | 8750 | OH2 | WAT | W | 584 | 28.884 | 69.807 | −39.556 | 1.00 | 21.83 | W |
| ATOM | 8751 | OH2 | WAT | W | 585 | 45.187 | 83.486 | −43.201 | 1.00 | 32.24 | W |
| ATOM | 8752 | OH2 | WAT | W | 586 | 29.669 | 81.398 | −42.870 | 1.00 | 30.89 | W |
| ATOM | 8753 | OH2 | WAT | W | 587 | 28.252 | 90.617 | −42.596 | 1.00 | 28.76 | W |
| ATOM | 8754 | OH2 | WAT | W | 588 | 19.626 | 84.323 | −28.580 | 1.00 | 37.20 | W |
| ATOM | 8755 | OH2 | WAT | W | 589 | 37.741 | 97.138 | −30.667 | 1.00 | 33.56 | W |
| ATOM | 8756 | OH2 | WAT | W | 590 | 40.566 | 44.263 | −31.708 | 1.00 | 35.51 | W |
| ATOM | 8757 | OH2 | WAT | W | 591 | 21.114 | 41.599 | 35.229 | 1.00 | 29.14 | W |
| ATOM | 8758 | OH2 | WAT | W | 592 | 39.247 | 30.347 | 13.105 | 1.00 | 38.89 | W |
| ATOM | 8759 | OH2 | WAT | W | 593 | 62.099 | 85.771 | −22.844 | 1.00 | 28.21 | W |
| ATOM | 8760 | OH2 | WAT | W | 594 | 31.040 | 68.457 | 14.098 | 1.00 | 24.91 | W |
| ATOM | 8761 | OH2 | WAT | W | 595 | 27.896 | 67.871 | 33.967 | 1.00 | 35.64 | W |
| ATOM | 8762 | OH2 | WAT | W | 596 | 15.003 | 63.544 | −25.860 | 1.00 | 25.42 | W |
| ATOM | 8763 | OH2 | WAT | W | 597 | 29.601 | 72.448 | 12.548 | 1.00 | 21.34 | W |
| ATOM | 8764 | OH2 | WAT | W | 599 | 61.559 | 92.883 | −38.650 | 1.00 | 36.64 | W |
| ATOM | 8765 | OH2 | WAT | W | 601 | 59.128 | 62.348 | 25.815 | 1.00 | 26.23 | W |
| ATOM | 8766 | OH2 | WAT | W | 602 | 51.205 | 79.279 | −1.713 | 1.00 | 23.39 | W |
| ATOM | 8767 | OH2 | WAT | W | 604 | 21.242 | 60.395 | 26.828 | 1.00 | 38.73 | W |
| ATOM | 8768 | OH2 | WAT | W | 605 | 59.833 | 90.941 | −35.483 | 1.00 | 27.48 | W |
| ATOM | 8769 | OH2 | WAT | W | 606 | 24.014 | 60.658 | 36.037 | 1.00 | 26.81 | W |
| ATOM | 8770 | OH2 | WAT | W | 607 | 11.603 | 61.649 | 0.313 | 1.00 | 32.24 | W |
| ATOM | 8771 | OH2 | WAT | W | 608 | 55.226 | 80.552 | −19.378 | 1.00 | 23.64 | W |
| ATOM | 8772 | OH2 | WAT | W | 609 | 16.743 | 74.434 | −28.596 | 1.00 | 34.41 | W |
| ATOM | 8773 | OH2 | WAT | W | 610 | 40.872 | 40.649 | −21.500 | 1.00 | 28.24 | W |
| ATOM | 8774 | OH2 | WAT | W | 611 | 28.668 | 68.441 | −46.081 | 1.00 | 39.60 | W |
| ATOM | 8775 | OH2 | WAT | W | 612 | 40.006 | 35.395 | 11.369 | 1.00 | 24.43 | W |
| ATOM | 8776 | OH2 | WAT | W | 613 | 18.904 | 80.509 | −11.812 | 1.00 | 29.72 | W |
| ATOM | 8777 | OH2 | WAT | W | 614 | 64.971 | 79.735 | −8.907 | 1.00 | 31.04 | W |
| ATOM | 8778 | OH2 | WAT | W | 615 | 72.774 | 43.611 | −12.245 | 1.00 | 33.51 | W |
| ATOM | 8779 | OH2 | WAT | W | 616 | 13.009 | 74.622 | −19.201 | 1.00 | 32.27 | W |
| ATOM | 8780 | OH2 | WAT | W | 617 | 57.062 | 75.479 | −2.931 | 1.00 | 23.61 | W |
| ATOM | 8781 | OH2 | WAT | W | 618 | 20.829 | 58.092 | 37.896 | 1.00 | 33.95 | W |
| ATOM | 8782 | OH2 | WAT | W | 619 | 50.615 | 77.218 | 2.234 | 1.00 | 30.34 | W |
| ATOM | 8783 | OH2 | WAT | W | 620 | 28.468 | 45.385 | 41.074 | 1.00 | 36.42 | W |
| ATOM | 8784 | OH2 | WAT | W | 621 | 23.139 | 36.879 | 13.360 | 1.00 | 24.96 | W |
| ATOM | 8785 | OH2 | WAT | W | 622 | 33.385 | 39.569 | −19.404 | 1.00 | 35.03 | W |
| ATOM | 8786 | OH2 | WAT | W | 623 | 33.787 | 49.363 | −15.050 | 1.00 | 32.82 | W |
| ATOM | 8787 | OH2 | WAT | W | 624 | 29.449 | 88.728 | −26.140 | 1.00 | 13.06 | W |
| ATOM | 8788 | OH2 | WAT | W | 625 | 37.151 | 33.801 | 5.746 | 1.00 | 33.81 | W |
| ATOM | 8789 | OH2 | WAT | W | 626 | 71.759 | 62.627 | −18.009 | 1.00 | 27.73 | W |
| ATOM | 8790 | OH2 | WAT | W | 627 | 34.323 | 70.260 | −44.782 | 1.00 | 32.70 | W |
| ATOM | 8791 | OH2 | WAT | W | 628 | 64.347 | 77.058 | 0.515 | 1.00 | 32.46 | W |
| ATOM | 8792 | OH2 | WAT | W | 630 | 35.947 | 55.944 | 8.521 | 1.00 | 41.57 | W |
| ATOM | 8793 | OH2 | WAT | W | 631 | 30.659 | 51.202 | 39.665 | 1.00 | 36.36 | W |
| ATOM | 8794 | OH2 | WAT | W | 632 | 54.265 | 57.827 | −13.764 | 1.00 | 37.73 | W |
| ATOM | 8795 | OH2 | WAT | W | 634 | 21.136 | 54.633 | 47.094 | 1.00 | 34.03 | W |
| ATOM | 8796 | OH2 | WAT | W | 635 | 14.453 | 50.762 | 1.021 | 1.00 | 26.77 | W |
| ATOM | 8797 | OH2 | WAT | W | 636 | 25.475 | 91.463 | −33.051 | 1.00 | 31.56 | W |
| ATOM | 8798 | OH2 | WAT | W | 637 | 73.185 | 71.227 | −29.293 | 1.00 | 31.95 | W |
| ATOM | 8799 | OH2 | WAT | W | 638 | 55.875 | 38.711 | 1.247 | 1.00 | 35.90 | W |
| ATOM | 8800 | OH2 | WAT | W | 639 | 37.492 | 83.840 | 4.429 | 1.00 | 33.70 | W |
| ATOM | 8801 | OH2 | WAT | W | 640 | 45.485 | 55.126 | 10.105 | 1.00 | 31.21 | W |
| ATOM | 8802 | OH2 | WAT | W | 642 | 66.889 | 81.336 | −11.141 | 1.00 | 29.67 | W |
| ATOM | 8803 | OH2 | WAT | W | 643 | 40.401 | 89.429 | −19.494 | 1.00 | 34.59 | W |
| ATOM | 8804 | OH2 | WAT | W | 644 | 43.726 | 56.577 | 33.715 | 1.00 | 23.89 | W |
| ATOM | 8805 | OH2 | WAT | W | 645 | 56.389 | 50.260 | −30.348 | 1.00 | 38.11 | W |
| ATOM | 8806 | OH2 | WAT | W | 646 | 63.345 | 57.663 | 25.731 | 1.00 | 33.20 | W |
| ATOM | 8807 | OH2 | WAT | W | 647 | 62.132 | 48.402 | −21.140 | 1.00 | 30.17 | W |
| ATOM | 8808 | OH2 | WAT | W | 648 | 62.795 | 59.511 | −22.094 | 1.00 | 43.84 | W |
| ATOM | 8809 | OH2 | WAT | W | 649 | 39.839 | 77.768 | 13.657 | 1.00 | 38.17 | W |
| ATOM | 8810 | OH2 | WAT | W | 650 | 26.708 | 91.704 | −27.698 | 1.00 | 27.71 | W |
| ATOM | 8811 | OH2 | WAT | W | 651 | 13.502 | 70.141 | −9.466 | 1.00 | 24.86 | W |
| ATOM | 8812 | OH2 | WAT | W | 652 | 60.670 | 78.924 | −41.587 | 1.00 | 24.16 | W |
| ATOM | 8813 | OH2 | WAT | W | 653 | 39.252 | 43.518 | 26.888 | 1.00 | 28.02 | W |
| ATOM | 8814 | OH2 | WAT | W | 654 | 11.931 | 70.822 | 2.485 | 1.00 | 31.13 | W |
| ATOM | 8815 | OH2 | WAT | W | 655 | 36.424 | 62.574 | −38.251 | 1.00 | 29.44 | W |
| ATOM | 8816 | OH2 | WAT | W | 656 | 76.055 | 83.157 | −25.276 | 1.00 | 39.00 | W |
| ATOM | 8817 | OH2 | WAT | W | 657 | 37.608 | 80.454 | −0.581 | 1.00 | 23.73 | W |
| ATOM | 8818 | OH2 | WAT | W | 658 | 47.350 | 53.378 | 35.765 | 1.00 | 33.39 | W |
| ATOM | 8819 | OH2 | WAT | W | 659 | 48.604 | 97.804 | −33.661 | 1.00 | 29.32 | W |
| ATOM | 8820 | OH2 | WAT | W | 660 | 63.595 | 75.084 | −45.655 | 1.00 | 33.19 | W |
| ATOM | 8821 | OH2 | WAT | W | 661 | 60.081 | 71.509 | 0.342 | 1.00 | 33.30 | W |
| ATOM | 8822 | OH2 | WAT | W | 662 | 41.130 | 48.174 | −29.424 | 1.00 | 33.56 | W |
| ATOM | 8823 | OH2 | WAT | W | 663 | 55.384 | 40.246 | −12.570 | 1.00 | 43.02 | W |
| ATOM | 8824 | OH2 | WAT | W | 664 | 74.977 | 83.935 | −22.956 | 1.00 | 33.05 | W |
| ATOM | 8825 | OH2 | WAT | W | 665 | 63.406 | 52.047 | −0.358 | 1.00 | 26.92 | W |
| ATOM | 8826 | OH2 | WAT | W | 666 | 37.221 | 36.481 | 18.964 | 1.00 | 39.54 | W |
| ATOM | 8827 | OH2 | WAT | W | 667 | 57.641 | 36.009 | −2.585 | 1.00 | 36.41 | W |
| ATOM | 8828 | OH2 | WAT | W | 668 | 23.115 | 48.814 | −36.297 | 1.00 | 32.39 | W |
| ATOM | 8829 | OH2 | WAT | W | 669 | 17.636 | 71.546 | −29.530 | 1.00 | 35.34 | W |
| ATOM | 8830 | OH2 | WAT | W | 670 | 30.674 | 40.378 | 38.143 | 1.00 | 36.19 | W |
| ATOM | 8831 | OH2 | WAT | W | 671 | 27.433 | 50.036 | 23.323 | 1.00 | 31.31 | W |
| ATOM | 8832 | OH2 | WAT | W | 672 | 30.557 | 48.124 | 40.431 | 1.00 | 36.97 | W |
| ATOM | 8833 | OH2 | WAT | W | 673 | 59.855 | 52.908 | −30.690 | 1.00 | 30.00 | W |
| ATOM | 8834 | OH2 | WAT | W | 674 | 44.853 | 40.592 | 13.345 | 1.00 | 29.68 | W |
| ATOM | 8835 | OH2 | WAT | W | 675 | 34.115 | 47.984 | 36.057 | 1.00 | 30.03 | W |
| ATOM | 8836 | OH2 | WAT | W | 676 | 27.358 | 82.247 | 3.089 | 1.00 | 28.86 | W |
| ATOM | 8837 | OH2 | WAT | W | 677 | 58.497 | 92.398 | −42.235 | 1.00 | 35.60 | W |
| ATOM | 8838 | OH2 | WAT | W | 678 | 49.253 | 52.427 | −37.624 | 1.00 | 38.08 | W |
| ATOM | 8839 | OH2 | WAT | W | 679 | 53.379 | 70.899 | −16.924 | 1.00 | 25.65 | W |
| ATOM | 8840 | OH2 | WAT | W | 680 | 57.626 | 62.695 | −15.503 | 1.00 | 35.96 | W |
| ATOM | 8841 | OH2 | WAT | W | 681 | 28.297 | 44.350 | −29.713 | 1.00 | 28.58 | W |
| ATOM | 8842 | OH2 | WAT | W | 682 | 48.852 | 49.432 | −38.154 | 1.00 | 36.59 | W |
| ATOM | 8843 | OH2 | WAT | W | 683 | 35.576 | 77.511 | −43.062 | 1.00 | 29.02 | W |
| ATOM | 8844 | OH2 | WAT | W | 684 | 9.623 | 58.535 | 17.852 | 1.00 | 33.51 | W |
| ATOM | 8845 | OH2 | WAT | W | 686 | 61.093 | 45.523 | −3.654 | 1.00 | 29.72 | W |
| ATOM | 8846 | OH2 | WAT | W | 687 | 65.483 | 78.684 | −5.700 | 1.00 | 27.73 | W |
| ATOM | 8847 | OH2 | WAT | W | 688 | 7.036 | 58.084 | 14.815 | 1.00 | 36.18 | W |
| ATOM | 8848 | OH2 | WAT | W | 689 | 49.432 | 50.626 | 32.574 | 1.00 | 27.09 | W |
| ATOM | 8849 | OH2 | WAT | W | 690 | 18.651 | 62.429 | 22.834 | 1.00 | 42.61 | W |
| ATOM | 8850 | OH2 | WAT | W | 691 | 30.258 | 34.015 | 21.108 | 1.00 | 34.14 | W |
| ATOM | 8851 | OH2 | WAT | W | 692 | 17.405 | 45.631 | −14.008 | 1.00 | 26.24 | W |
| ATOM | 8852 | OH2 | WAT | W | 693 | 28.725 | 55.316 | 39.985 | 1.00 | 27.55 | W |
| ATOM | 8853 | OH2 | WAT | W | 700 | 50.875 | 59.946 | −8.928 | 1.00 | 11.46 | W |
| ATOM | 8854 | OH2 | WAT | W | 701 | 43.729 | 63.036 | −28.635 | 1.00 | 10.90 | W |
| ATOM | 8855 | OH2 | WAT | W | 702 | 38.088 | 81.789 | −31.356 | 1.00 | 11.61 | W |
| ATOM | 8856 | OH2 | WAT | W | 703 | 32.349 | 39.151 | −4.925 | 1.00 | 10.69 | W |
| ATOM | 8857 | OH2 | WAT | W | 705 | 60.345 | 60.974 | −18.133 | 1.00 | 10.00 | W |
| ATOM | 8858 | OH2 | WAT | W | 706 | 63.195 | 58.590 | −1.434 | 1.00 | 14.50 | W |
| ATOM | 8859 | OH2 | WAT | W | 707 | 60.694 | 60.822 | −3.596 | 1.00 | 12.04 | W |
| ATOM | 8860 | OH2 | WAT | W | 708 | 46.601 | 74.399 | 7.782 | 1.00 | 12.30 | W |
| ATOM | 8861 | OH2 | WAT | W | 709 | 54.966 | 57.537 | −0.483 | 1.00 | 13.73 | W |
| ATOM | 8862 | OH2 | WAT | W | 710 | 30.225 | 74.877 | −29.275 | 1.00 | 13.65 | W |
| ATOM | 8863 | OH2 | WAT | W | 712 | 58.878 | 56.724 | −1.422 | 1.00 | 11.48 | W |
| ATOM | 8864 | OH2 | WAT | W | 713 | 58.700 | 52.597 | −0.079 | 1.00 | 14.33 | W |
| ATOM | 8865 | OH2 | WAT | W | 714 | 63.107 | 61.088 | −4.839 | 1.00 | 12.89 | W |
| ATOM | 8866 | OH2 | WAT | W | 716 | 27.955 | 48.693 | 12.047 | 1.00 | 17.48 | W |
| ATOM | 8867 | OH2 | WAT | W | 717 | 66.623 | 56.169 | −20.267 | 1.00 | 17.13 | W |
| ATOM | 8868 | OH2 | WAT | W | 718 | 19.936 | 46.755 | −14.350 | 1.00 | 16.77 | W |
| ATOM | 8869 | OH2 | WAT | W | 719 | 38.627 | 69.291 | −38.085 | 1.00 | 17.45 | W |
| ATOM | 8870 | OH2 | WAT | W | 720 | 33.604 | 60.216 | 23.237 | 1.00 | 13.28 | W |
| ATOM | 8871 | OH2 | WAT | W | 721 | 39.652 | 55.611 | 3.022 | 1.00 | 13.93 | W |
| ATOM | 8872 | OH2 | WAT | W | 722 | 24.646 | 61.455 | 9.397 | 1.00 | 14.96 | W |
| ATOM | 8873 | OH2 | WAT | W | 723 | 16.774 | 57.081 | 13.004 | 1.00 | 14.37 | W |
| ATOM | 8874 | OH2 | WAT | W | 724 | 39.450 | 69.183 | −35.373 | 1.00 | 14.13 | W |
| ATOM | 8875 | OH2 | WAT | W | 725 | 49.892 | 67.527 | 4.244 | 1.00 | 18.93 | W |
| ATOM | 8876 | OH2 | WAT | W | 726 | 27.672 | 53.307 | 20.113 | 1.00 | 14.50 | W |
| ATOM | 8877 | OH2 | WAT | W | 728 | 29.774 | 83.030 | 2.442 | 1.00 | 22.49 | W |
| ATOM | 8878 | OH2 | WAT | W | 729 | 28.084 | 90.857 | −25.245 | 1.00 | 18.36 | W |
| ATOM | 8879 | OH2 | WAT | W | 730 | 46.074 | 77.662 | 9.467 | 1.00 | 18.68 | W |
| ATOM | 8880 | OH2 | WAT | W | 732 | 12.563 | 49.413 | 17.711 | 1.00 | 22.37 | W |
| ATOM | 8881 | OH2 | WAT | W | 733 | 81.206 | 68.406 | −11.410 | 1.00 | 22.16 | W |
| ATOM | 8882 | OH2 | WAT | W | 734 | 41.474 | 44.788 | −6.895 | 1.00 | 14.61 | W |
| ATOM | 8883 | OH2 | WAT | W | 735 | 45.595 | 67.851 | −27.467 | 1.00 | 21.17 | W |
| ATOM | 8884 | OH2 | WAT | W | 736 | 14.207 | 50.502 | 24.242 | 1.00 | 21.49 | W |
| ATOM | 8885 | OH2 | WAT | W | 737 | 67.655 | 46.510 | −1.449 | 1.00 | 22.53 | W |
| ATOM | 8886 | OH2 | WAT | W | 738 | 9.070 | 50.439 | 8.116 | 1.00 | 23.35 | W |
| ATOM | 8887 | OH2 | WAT | W | 739 | 48.508 | 40.902 | −1.043 | 1.00 | 22.37 | W |
| ATOM | 8888 | OH2 | WAT | W | 740 | 17.771 | 74.445 | 3.962 | 1.00 | 24.93 | W |
| ATOM | 8889 | OH2 | WAT | W | 741 | 44.945 | 55.093 | −18.224 | 1.00 | 23.58 | W |
| ATOM | 8890 | OH2 | WAT | W | 742 | 83.440 | 66.338 | −15.493 | 1.00 | 19.93 | W |
| ATOM | 8891 | OH2 | WAT | W | 743 | 48.974 | 69.111 | −36.939 | 1.00 | 23.36 | W |
| ATOM | 8892 | OH2 | WAT | W | 744 | 51.693 | 70.185 | −38.125 | 1.00 | 23.72 | W |
| ATOM | 8893 | OH2 | WAT | W | 745 | 29.695 | 72.422 | 34.299 | 1.00 | 24.92 | W |
| ATOM | 8894 | OH2 | WAT | W | 746 | 37.507 | 61.422 | −32.436 | 1.00 | 21.97 | W |
| ATOM | 8895 | OH2 | WAT | W | 747 | 57.372 | 48.994 | 21.252 | 1.00 | 21.06 | W |
| ATOM | 8896 | OH2 | WAT | W | 748 | 42.963 | 59.488 | −31.444 | 1.00 | 21.62 | W |
| ATOM | 8897 | OH2 | WAT | W | 749 | 32.019 | 34.321 | −3.215 | 1.00 | 21.00 | W |
| ATOM | 8898 | OH2 | WAT | W | 750 | 37.070 | 59.043 | −33.539 | 1.00 | 21.08 | W |
| ATOM | 8899 | OH2 | WAT | W | 751 | 61.487 | 59.205 | 14.822 | 1.00 | 24.82 | W |
| ATOM | 8900 | OH2 | WAT | W | 752 | 40.156 | 56.207 | −19.111 | 1.00 | 25.71 | W |
| ATOM | 8901 | OH2 | WAT | W | 753 | 47.525 | 87.559 | −44.311 | 1.00 | 48.44 | W |
| ATOM | 8902 | OH2 | WAT | W | 754 | 61.661 | 76.348 | −10.460 | 1.00 | 21.23 | W |
| ATOM | 8903 | OH2 | WAT | W | 755 | 79.392 | 50.972 | −11.546 | 1.00 | 39.61 | W |
| ATOM | 8904 | OH2 | WAT | W | 756 | 37.676 | 51.645 | −18.478 | 1.00 | 18.77 | W |
| ATOM | 8905 | OH2 | WAT | W | 757 | 25.691 | 52.103 | 13.079 | 1.00 | 23.59 | W |
| ATOM | 8906 | OH2 | WAT | W | 758 | 69.687 | 63.958 | −28.517 | 1.00 | 25.13 | W |
| ATOM | 8907 | OH2 | WAT | W | 759 | 73.007 | 66.604 | −9.978 | 1.00 | 21.68 | W |
| ATOM | 8908 | OH2 | WAT | W | 760 | 42.818 | 56.438 | 2.724 | 1.00 | 17.92 | W |
| ATOM | 8909 | OH2 | WAT | W | 761 | 56.051 | 65.114 | −36.107 | 1.00 | 19.42 | W |
| ATOM | 8910 | OH2 | WAT | W | 762 | 17.857 | 66.012 | 27.868 | 1.00 | 26.73 | W |
| ATOM | 8911 | OH2 | WAT | W | 763 | 11.781 | 63.583 | −16.941 | 1.00 | 21.03 | W |
| ATOM | 8912 | OH2 | WAT | W | 764 | 35.221 | 86.295 | −17.790 | 1.00 | 27.96 | W |
| ATOM | 8913 | OH2 | WAT | W | 766 | 9.801 | 60.334 | −20.514 | 1.00 | 24.44 | W |
| ATOM | 8914 | OH2 | WAT | W | 767 | 11.577 | 48.702 | −2.429 | 1.00 | 28.25 | W |
| ATOM | 8915 | OH2 | WAT | W | 768 | 40.633 | 56.964 | 31.024 | 1.00 | 28.09 | W |
| ATOM | 8916 | OH2 | WAT | W | 769 | 39.812 | 52.932 | 36.459 | 1.00 | 28.11 | W |
| ATOM | 8917 | OH2 | WAT | W | 770 | 49.931 | 47.339 | 28.976 | 1.00 | 22.00 | W |
| ATOM | 8918 | OH2 | WAT | W | 771 | 24.098 | 34.395 | 15.460 | 1.00 | 23.97 | W |
| ATOM | 8919 | OH2 | WAT | W | 772 | 59.487 | 50.889 | 24.775 | 1.00 | 26.83 | W |
| ATOM | 8920 | OH2 | WAT | W | 773 | 38.030 | 83.105 | −1.002 | 1.00 | 29.02 | W |
| ATOM | 8921 | OH2 | WAT | W | 775 | 12.961 | 51.685 | −12.118 | 1.00 | 24.44 | W |
| ATOM | 8922 | OH2 | WAT | W | 776 | 48.180 | 50.593 | 35.188 | 1.00 | 27.91 | W |
| ATOM | 8923 | OH2 | WAT | W | 777 | 48.088 | 41.570 | −5.478 | 1.00 | 25.82 | W |
| ATOM | 8924 | OH2 | WAT | W | 778 | 22.201 | 40.755 | −11.309 | 1.00 | 22.86 | W |
| ATOM | 8925 | OH2 | WAT | W | 779 | 16.035 | 36.324 | 16.220 | 1.00 | 33.87 | W |
| ATOM | 8926 | OH2 | WAT | W | 780 | 22.449 | 68.096 | 20.372 | 1.00 | 25.52 | W |
| ATOM | 8927 | OH2 | WAT | W | 781 | 72.144 | 47.265 | −17.924 | 1.00 | 27.21 | W |
| ATOM | 8928 | OH2 | WAT | W | 782 | 40.774 | 85.239 | −11.931 | 1.00 | 23.09 | W |
| ATOM | 8929 | OH2 | WAT | W | 783 | 56.758 | 46.693 | 15.829 | 1.00 | 26.36 | W |
| ATOM | 8930 | OH2 | WAT | W | 784 | 51.826 | 63.844 | 24.459 | 1.00 | 25.35 | W |
| ATOM | 8931 | OH2 | WAT | W | 785 | 56.613 | 58.444 | 1.389 | 1.00 | 27.95 | W |
| ATOM | 8932 | OH2 | WAT | W | 786 | 57.457 | 58.417 | 5.430 | 1.00 | 28.29 | W |
| ATOM | 8933 | OH2 | WAT | W | 787 | 75.229 | 64.103 | −16.398 | 1.00 | 25.10 | W |
| ATOM | 8934 | OH2 | WAT | W | 788 | 14.676 | 70.516 | −23.121 | 1.00 | 24.85 | W |
| ATOM | 8935 | OH2 | WAT | W | 789 | 13.074 | 52.164 | −15.015 | 1.00 | 26.93 | W |
| ATOM | 8936 | OH2 | WAT | W | 790 | 13.077 | 61.896 | −25.010 | 1.00 | 28.53 | W |
| ATOM | 8937 | OH2 | WAT | W | 791 | 23.191 | 87.067 | −32.614 | 1.00 | 29.37 | W |
| ATOM | 8938 | OH2 | WAT | W | 792 | 66.312 | 81.679 | −14.185 | 1.00 | 25.05 | W |
| ATOM | 8939 | OH2 | WAT | W | 793 | 47.318 | 61.469 | −28.719 | 1.00 | 27.79 | W |
| ATOM | 8940 | OH2 | WAT | W | 794 | 52.473 | 70.852 | 5.989 | 1.00 | 28.42 | W |
| ATOM | 8941 | OH2 | WAT | W | 795 | 40.973 | 55.861 | −33.310 | 1.00 | 27.84 | w |
| ATOM | 8942 | OH2 | WAT | W | 796 | 30.245 | 66.418 | 10.988 | 1.00 | 25.30 | W |
| ATOM | 8943 | OH2 | WAT | W | 797 | 39.668 | 39.709 | 19.992 | 1.00 | 28.66 | W |
| ATOM | 8944 | OH2 | WAT | W | 798 | 44.704 | 55.276 | 36.258 | 1.00 | 32.18 | W |
| ATOM | 8945 | OH2 | WAT | W | 799 | 13.966 | 62.480 | 14.594 | 1.00 | 26.13 | W |
| ATOM | 8946 | OH2 | WAT | W | 800 | 40.024 | 39.702 | −0.423 | 1.00 | 27.38 | W |
| ATOM | 8947 | OH2 | WAT | W | 801 | 32.549 | 69.495 | 22.160 | 1.00 | 28.57 | W |
| ATOM | 8948 | OH2 | WAT | W | 802 | 41.883 | 44.124 | −11.770 | 1.00 | 25.88 | W |
| ATOM | 8949 | OH2 | WAT | W | 803 | 26.947 | 41.616 | −27.654 | 1.00 | 30.54 | W |
| ATOM | 8950 | OH2 | WAT | W | 804 | 19.843 | 41.627 | −5.057 | 1.00 | 25.74 | W |
| ATOM | 8951 | OH2 | WAT | W | 805 | 13.685 | 60.590 | 7.622 | 1.00 | 25.20 | W |
| ATOM | 8952 | OH2 | WAT | W | 806 | 46.720 | 45.811 | 11.549 | 1.00 | 29.52 | W |
| ATOM | 8953 | OH2 | WAT | W | 807 | 42.921 | 68.593 | −29.946 | 1.00 | 27.18 | W |
| ATOM | 8954 | OH2 | WAT | W | 808 | 24.412 | 53.888 | −8.318 | 1.00 | 33.09 | W |
| ATOM | 8955 | OH2 | WAT | W | 809 | 47.609 | 69.894 | −16.650 | 1.00 | 25.75 | W |
| ATOM | 8956 | OH2 | WAT | W | 810 | 58.802 | 93.579 | −27.521 | 1.00 | 28.20 | W |
| ATOM | 8957 | OH2 | WAT | W | 811 | 31.338 | 79.105 | −42.859 | 1.00 | 29.67 | W |
| ATOM | 8958 | OH2 | WAT | W | 812 | 22.092 | 42.668 | −13.217 | 1.00 | 24.02 | W |
| ATOM | 8959 | OH2 | WAT | W | 813 | 52.770 | 92.046 | −23.358 | 1.00 | 26.46 | W |
| ATOM | 8960 | OH2 | WAT | W | 814 | 60.321 | 50.996 | 20.144 | 1.00 | 31.94 | W |
| ATOM | 8961 | OH2 | WAT | W | 815 | 60.777 | 56.054 | 2.005 | 1.00 | 27.67 | W |
| ATOM | 8962 | OH2 | WAT | W | 816 | 19.477 | 58.541 | −36.746 | 1.00 | 23.57 | W |
| ATOM | 8963 | OH2 | WAT | W | 817 | 30.153 | 49.938 | 12.462 | 1.00 | 54.57 | W |
| ATOM | 8964 | OH2 | WAT | W | 818 | 26.284 | 66.727 | 19.939 | 1.00 | 26.81 | W |
| ATOM | 8965 | OH2 | WAT | W | 819 | 11.971 | 41.446 | 19.843 | 1.00 | 29.18 | W |
| ATOM | 8966 | OH2 | WAT | W | 820 | 69.267 | 77.131 | −34.141 | 1.00 | 25.35 | W |
| ATOM | 8967 | OH2 | WAT | W | 821 | 16.589 | 42.916 | −2.539 | 1.00 | 26.70 | W |
| ATOM | 8968 | OH2 | WAT | W | 822 | 35.924 | 59.889 | −37.655 | 1.00 | 33.22 | W |
| ATOM | 8969 | OH2 | WAT | W | 823 | 34.054 | 66.190 | −42.550 | 1.00 | 34.74 | W |
| ATOM | 8970 | OH2 | WAT | W | 824 | 57.237 | 36.102 | −14.386 | 1.00 | 32.76 | W |
| ATOM | 8971 | OH2 | WAT | W | 825 | 37.157 | 37.518 | 2.919 | 1.00 | 29.99 | W |
| ATOM | 8972 | OH2 | WAT | W | 826 | 51.214 | 66.642 | 8.544 | 1.00 | 21.97 | W |
| ATOM | 8973 | OH2 | WAT | W | 829 | 10.791 | 51.469 | 21.342 | 1.00 | 26.06 | W |
| ATOM | 8974 | OH2 | WAT | W | 830 | 30.338 | 87.215 | −14.953 | 1.00 | 24.37 | W |
| ATOM | 8975 | OH2 | WAT | W | 831 | 42.244 | 54.832 | −17.439 | 1.00 | 27.73 | W |
| ATOM | 8976 | OH2 | WAT | W | 832 | 48.355 | 76.393 | 8.326 | 1.00 | 29.41 | W |
| ATOM | 8977 | OH2 | WAT | W | 833 | 29.663 | 76.034 | 10.889 | 1.00 | 28.76 | W |
| ATOM | 8978 | OH2 | WAT | W | 834 | 43.010 | 98.114 | −37.134 | 1.00 | 31.67 | W |
| ATOM | 8979 | OH2 | WAT | W | 835 | 24.455 | 65.295 | 9.661 | 1.00 | 27.20 | W |
| ATOM | 8980 | OH2 | WAT | W | 836 | 64.682 | 78.684 | −1.585 | 1.00 | 26.20 | W |
| ATOM | 8981 | OH2 | WAT | W | 837 | 43.254 | 40.464 | −22.987 | 1.00 | 27.81 | W |
| ATOM | 8982 | OH2 | WAT | W | 838 | 59.455 | 49.064 | 2.200 | 1.00 | 31.34 | W |
| ATOM | 8983 | OH2 | WAT | W | 839 | 68.991 | 41.685 | −17.211 | 1.00 | 28.54 | W |
| ATOM | 8984 | OH2 | WAT | W | 840 | 24.783 | 71.648 | −39.983 | 1.00 | 23.32 | W |
| ATOM | 8985 | OH2 | WAT | W | 841 | 23.732 | 48.420 | −24.123 | 1.00 | 30.99 | W |
| ATOM | 8986 | OH2 | WAT | W | 842 | 46.974 | 68.268 | −24.515 | 1.00 | 24.37 | W |
| ATOM | 8987 | OH2 | WAT | W | 843 | 53.359 | 53.304 | −30.034 | 1.00 | 30.89 | W |
| ATOM | 8988 | OH2 | WAT | W | 844 | 24.825 | 34.332 | 35.789 | 1.00 | 31.04 | W |
| ATOM | 8989 | OH2 | WAT | W | 845 | 13.297 | 52.427 | 25.888 | 1.00 | 29.86 | W |
| ATOM | 8990 | OH2 | WAT | W | 846 | 51.677 | 44.456 | 2.991 | 1.00 | 29.74 | W |
| ATOM | 8991 | OH2 | WAT | W | 847 | 21.471 | 55.374 | 39.891 | 1.00 | 35.10 | W |
| ATOM | 8992 | OH2 | WAT | W | 848 | 64.222 | 70.580 | −5.272 | 1.00 | 32.06 | W |
| ATOM | 8993 | OH2 | WAT | W | 849 | 46.741 | 89.756 | −23.288 | 1.00 | 33.91 | W |
| ATOM | 8994 | OH2 | WAT | W | 850 | 8.118 | 53.068 | −7.667 | 1.00 | 30.58 | W |
| ATOM | 8995 | OH2 | WAT | W | 851 | 22.086 | 80.693 | −42.196 | 1.00 | 29.19 | W |
| ATOM | 8996 | OH2 | WAT | W | 852 | 63.873 | 65.397 | −30.493 | 1.00 | 41.52 | W |
| ATOM | 8997 | OH2 | WAT | W | 853 | 18.598 | 48.634 | −19.927 | 1.00 | 32.70 | W |
| ATOM | 8998 | OH2 | WAT | W | 854 | 59.272 | 76.946 | −4.206 | 1.00 | 31.52 | W |
| ATOM | 8999 | OH2 | WAT | W | 855 | 21.016 | 83.897 | −12.178 | 1.00 | 32.20 | W |
| ATOM | 9000 | OH2 | WAT | W | 856 | 55.515 | 67.407 | 13.403 | 1.00 | 41.93 | W |
| ATOM | 9001 | OH2 | WAT | W | 857 | 19.239 | 52.193 | −31.228 | 1.00 | 36.10 | W |
| ATOM | 9002 | OH2 | WAT | W | 858 | 67.372 | 85.521 | −35.959 | 1.00 | 35.41 | W |
| ATOM | 9003 | OH2 | WAT | W | 859 | 29.632 | 74.968 | 22.211 | 1.00 | 25.23 | W |
| ATOM | 9004 | OH2 | WAT | W | 860 | 32.821 | 84.208 | 0.305 | 1.00 | 28.56 | W |
| ATOM | 9005 | OH2 | WAT | W | 861 | 45.757 | 65.469 | −28.604 | 1.00 | 29.35 | W |
| ATOM | 9006 | OH2 | WAT | W | 862 | 12.983 | 62.062 | −2.298 | 1.00 | 27.17 | W |
| ATOM | 9007 | OH2 | WAT | W | 864 | 25.739 | 63.240 | 13.712 | 1.00 | 27.83 | W |
| ATOM | 9008 | OH2 | WAT | W | 865 | 28.531 | 34.868 | 32.152 | 1.00 | 30.38 | W |
| ATOM | 9009 | OH2 | WAT | W | 866 | 35.763 | 68.453 | 23.134 | 1.00 | 30.35 | W |
| ATOM | 9010 | OH2 | WAT | W | 867 | 31.861 | 33.726 | 17.240 | 1.00 | 35.41 | W |
| ATOM | 9011 | OH2 | WAT | W | 868 | 41.587 | 84.247 | −2.302 | 1.00 | 34.07 | W |
| ATOM | 9012 | OH2 | WAT | W | 869 | 39.915 | 100.019 | −31.828 | 1.00 | 38.43 | W |
| ATOM | 9013 | OH2 | WAT | W | 870 | 28.436 | 63.613 | 10.869 | 1.00 | 28.44 | W |
| ATOM | 9014 | OH2 | WAT | W | 871 | 54.801 | 82.230 | −44.567 | 1.00 | 37.79 | W |
| ATOM | 9015 | OH2 | WAT | W | 872 | 69.905 | 53.571 | −10.383 | 1.00 | 29.60 | W |
| ATOM | 9016 | OH2 | WAT | W | 873 | 36.928 | 79.069 | −2.526 | 1.00 | 44.59 | W |
| ATOM | 9017 | OH2 | WAT | W | 874 | 12.247 | 64.242 | −12.633 | 1.00 | 28.33 | W |
| ATOM | 9018 | OH2 | WAT | W | 875 | 39.300 | 50.117 | 36.307 | 1.00 | 33.21 | W |
| ATOM | 9019 | OH2 | WAT | W | 876 | 28.044 | 44.594 | 38.197 | 1.00 | 29.80 | W |
| ATOM | 9020 | OH2 | WAT | W | 877 | 27.720 | 38.687 | −22.666 | 1.00 | 30.70 | W |
| ATOM | 9021 | OH2 | WAT | W | 878 | 72.387 | 95.667 | −31.128 | 1.00 | 32.23 | W |
| ATOM | 9022 | OH2 | WAT | W | 879 | 76.363 | 46.945 | −11.610 | 1.00 | 34.32 | W |
| ATOM | 9023 | OH2 | WAT | W | 880 | 21.492 | 48.972 | −38.837 | 1.00 | 33.31 | W |
| ATOM | 9024 | OH2 | WAT | W | 881 | 23.344 | 74.976 | −30.166 | 1.00 | 33.88 | W |
| ATOM | 9025 | OH2 | WAT | W | 882 | 20.850 | 31.662 | 31.576 | 1.00 | 30.80 | W |
| ATOM | 9026 | OH2 | WAT | W | 883 | 29.614 | 42.294 | −28.740 | 1.00 | 39.46 | W |
| ATOM | 9027 | OH2 | WAT | W | 884 | 42.804 | 47.450 | −27.321 | 1.00 | 34.86 | W |
| ATOM | 9028 | OH2 | WAT | W | 885 | 55.814 | 51.939 | 27.423 | 1.00 | 30.74 | W |
| ATOM | 9029 | OH2 | WAT | W | 886 | 31.683 | 58.884 | −40.938 | 1.00 | 50.66 | W |
| ATOM | 9030 | OH2 | WAT | W | 887 | 18.480 | 42.922 | 27.406 | 1.00 | 25.20 | W |
| ATOM | 9031 | OH2 | WAT | W | 888 | 62.075 | 81.573 | −44.200 | 1.00 | 40.76 | W |
| ATOM | 9032 | OH2 | WAT | W | 889 | 7.465 | 54.408 | 17.306 | 1.00 | 30.35 | W |
| ATOM | 9033 | OH2 | WAT | W | 890 | 18.101 | 41.684 | 25.245 | 1.00 | 32.73 | W |
| ATOM | 9034 | OH2 | WAT | W | 891 | 27.429 | 82.562 | 5.738 | 1.00 | 36.27 | W |
| ATOM | 9035 | OH2 | WAT | W | 892 | 38.868 | 53.058 | 1.997 | 1.00 | 30.70 | W |
| ATOM | 9036 | OH2 | WAT | W | 893 | 12.036 | 67.258 | 2.652 | 1.00 | 42.94 | W |
| ATOM | 9037 | OH2 | WAT | W | 894 | 4.469 | 56.610 | −6.387 | 1.00 | 29.55 | W |
| ATOM | 9038 | OH2 | WAT | W | 895 | 21.857 | 76.782 | 8.689 | 1.00 | 31.52 | W |
| ATOM | 9039 | OH2 | WAT | W | 896 | 25.676 | 87.966 | −42.799 | 1.00 | 39.01 | W |
| ATOM | 9040 | OH2 | WAT | W | 897 | 10.540 | 62.431 | −12.196 | 1.00 | 31.66 | W |
| ATOM | 9041 | OH2 | WAT | W | 898 | 44.607 | 72.169 | −44.474 | 1.00 | 32.45 | W |
| ATOM | 9042 | OH2 | WAT | W | 899 | 37.212 | 39.898 | 26.577 | 1.00 | 34.20 | W |
| ATOM | 9043 | OH2 | WAT | W | 900 | 23.165 | 88.711 | −37.906 | 1.00 | 35.10 | W |
| ATOM | 9044 | OH2 | WAT | W | 901 | 70.029 | 62.747 | −24.229 | 1.00 | 35.37 | W |
| ATOM | 9045 | OH2 | WAT | W | 902 | 23.331 | 65.414 | 18.401 | 1.00 | 33.45 | W |
| ATOM | 9046 | OH2 | WAT | W | 903 | 67.038 | 38.543 | −9.645 | 1.00 | 29.50 | W |
| ATOM | 9047 | OH2 | WAT | W | 904 | 65.467 | 87.593 | −41.084 | 1.00 | 36.40 | W |
| ATOM | 9048 | OH2 | WAT | W | 905 | 14.017 | 43.333 | 28.865 | 1.00 | 35.15 | W |
| ATOM | 9049 | OH2 | WAT | W | 906 | 13.469 | 48.254 | 1.397 | 1.00 | 37.34 | W |
| ATOM | 9050 | OH2 | WAT | W | 907 | 55.436 | 69.137 | −37.827 | 1.00 | 30.40 | W |
| ATOM | 9051 | OH2 | WAT | W | 908 | 26.966 | 47.556 | −23.954 | 1.00 | 29.40 | W |
| ATOM | 9052 | OH2 | WAT | W | 909 | 69.728 | 82.023 | −33.947 | 1.00 | 32.27 | W |
| ATOM | 9053 | OH2 | WAT | W | 910 | 11.998 | 62.183 | 22.815 | 1.00 | 38.70 | W |
| ATOM | 9054 | OH2 | WAT | W | 911 | 30.793 | 29.732 | 5.534 | 1.00 | 37.65 | W |
| ATOM | 9055 | OH2 | WAT | W | 912 | 19.179 | 36.459 | 22.322 | 1.00 | 40.49 | W |
| ATOM | 9056 | OH2 | WAT | W | 913 | 27.736 | 86.412 | −14.732 | 1.00 | 31.73 | W |
| ATOM | 9057 | OH2 | WAT | W | 914 | 66.117 | 50.416 | 0.413 | 1.00 | 30.39 | W |
| ATOM | 9058 | OH2 | WAT | W | 915 | 28.492 | 63.881 | 13.983 | 1.00 | 29.55 | W |
| ATOM | 9059 | OH2 | WAT | W | 916 | 51.974 | 60.105 | 28.076 | 1.00 | 30.69 | W |
| ATOM | 9060 | OH2 | WAT | W | 917 | 52.332 | 72.483 | 18.319 | 1.00 | 29.21 | W |
| ATOM | 9061 | OH2 | WAT | W | 918 | 47.072 | 74.947 | −26.272 | 1.00 | 30.57 | W |
| ATOM | 9062 | OH2 | WAT | W | 919 | 65.371 | 81.208 | −4.746 | 1.00 | 32.50 | W |
| ATOM | 9063 | OH2 | WAT | W | 920 | 48.492 | 72.168 | −26.185 | 1.00 | 32.28 | W |
| ATOM | 9064 | OH2 | WAT | W | 921 | 59.945 | 68.604 | −38.880 | 1.00 | 29.65 | W |
| ATOM | 9065 | OH2 | WAT | W | 922 | 22.864 | 85.051 | −16.984 | 1.00 | 38.49 | W |
| ATOM | 9066 | OH2 | WAT | W | 923 | 25.956 | 76.250 | −38.830 | 1.00 | 30.40 | W |
| ATOM | 9067 | OH2 | WAT | W | 924 | 20.018 | 73.946 | −28.969 | 1.00 | 42.04 | W |
| ATOM | 9068 | OH2 | WAT | W | 925 | 59.188 | 55.422 | 27.104 | 1.00 | 33.12 | W |
| ATOM | 9069 | OH2 | WAT | W | 926 | 44.334 | 87.421 | −15.927 | 1.00 | 42.21 | W |
| ATOM | 9070 | OH2 | WAT | W | 927 | 64.029 | 76.889 | −40.494 | 1.00 | 32.61 | W |
| ATOM | 9071 | OH2 | WAT | W | 928 | 14.068 | 58.271 | 16.406 | 1.00 | 21.84 | W |
| ATOM | 9072 | OH2 | WAT | W | 929 | 13.929 | 57.640 | 19.009 | 1.00 | 21.02 | W |
| ATOM | 9073 | OH2 | WAT | W | 930 | 24.125 | 63.343 | 11.562 | 1.00 | 29.26 | W |
| ATOM | 9074 | OH2 | WAT | W | 931 | 28.117 | 39.644 | 11.297 | 1.00 | 15.34 | W |
| ATOM | 9075 | OH2 | WAT | W | 932 | 28.948 | 81.833 | −35.632 | 1.00 | 24.97 | W |
| ATOM | 9076 | OH2 | WAT | W | 1000 | 12.359 | 55.778 | −15.197 | 1.00 | 19.33 | W |
| ATOM | 9077 | OH2 | WAT | W | 1001 | 81.546 | 66.995 | −13.758 | 1.00 | 21.02 | W |
| ATOM | 9078 | OH2 | WAT | W | 1002 | 59.050 | 58.105 | 0.971 | 1.00 | 20.10 | W |
| ATOM | 9079 | OH2 | WAT | W | 1003 | 49.194 | 68.129 | 6.727 | 1.00 | 24.02 | W |
| ATOM | 9080 | OH2 | WAT | W | 1004 | 68.412 | 79.523 | −28.528 | 1.00 | 22.06 | W |
| ATOM | 9081 | OH2 | WAT | W | 1005 | 26.119 | 32.717 | 28.755 | 1.00 | 25.06 | W |
| ATOM | 9082 | OH2 | WAT | W | 1006 | 32.842 | 69.360 | 24.703 | 1.00 | 23.57 | W |
| ATOM | 9083 | OH2 | WAT | W | 1007 | 27.842 | 102.244 | −19.194 | 1.00 | 26.24 | W |
| ATOM | 9084 | OH2 | WAT | W | 1008 | 71.801 | 73.870 | −29.215 | 1.00 | 26.76 | W |
| ATOM | 9085 | OH2 | WAT | W | 1009 | 79.342 | 50.598 | −8.845 | 1.00 | 28.86 | W |
| ATOM | 9086 | OH2 | WAT | W | 1010 | 37.052 | 83.691 | −3.974 | 1.00 | 26.37 | W |
| ATOM | 9087 | OH2 | WAT | W | 1011 | 59.408 | 51.958 | 2.554 | 1.00 | 28.10 | W |
| ATOM | 9088 | OH2 | WAT | W | 1012 | 19.601 | 84.239 | −18.370 | 1.00 | 28.43 | W |
| ATOM | 9089 | OH2 | WAT | W | 1013 | 43.956 | 79.243 | −21.470 | 1.00 | 32.67 | W |
| ATOM | 9090 | OH2 | WAT | W | 1014 | 58.023 | 52.829 | 26.220 | 1.00 | 28.82 | W |
| ATOM | 9091 | OH2 | WAT | W | 1015 | 17.061 | 57.454 | −36.404 | 1.00 | 28.26 | W |
| ATOM | 9092 | OH2 | WAT | W | 1016 | 46.665 | 43.031 | 9.625 | 1.00 | 30.00 | W |
| ATOM | 9093 | OH2 | WAT | W | 1017 | 27.197 | 72.121 | 34.046 | 1.00 | 34.22 | W |
| ATOM | 9094 | OH2 | WAT | W | 1018 | 7.536 | 54.149 | −10.230 | 1.00 | 25.55 | W |
| ATOM | 9095 | OH2 | WAT | W | 1019 | 24.878 | 52.245 | 43.415 | 1.00 | 40.21 | W |
| ATOM | 9096 | OH2 | WAT | W | 1020 | 49.909 | 46.879 | −30.749 | 1.00 | 31.80 | W |
| ATOM | 9097 | OH2 | WAT | W | 1021 | 42.581 | 78.539 | −29.540 | 1.00 | 35.27 | W |
| ATOM | 9098 | OH2 | WAT | W | 1022 | 27.591 | 88.817 | −12.263 | 1.00 | 30.74 | W |
| ATOM | 9099 | OH2 | WAT | W | 1023 | 56.603 | 89.918 | −43.608 | 1.00 | 30.70 | W |
| ATOM | 9100 | OH2 | WAT | W | 1024 | 13.964 | 44.811 | 26.794 | 1.00 | 36.36 | W |
| ATOM | 9101 | OH2 | WAT | W | 1025 | 69.388 | 83.796 | −35.992 | 1.00 | 27.38 | W |
| ATOM | 9102 | OH2 | WAT | W | 1026 | 57.993 | 93.302 | −24.988 | 1.00 | 27.86 | W |
| ATOM | 9103 | OH2 | WAT | W | 1027 | 64.598 | 70.559 | −7.944 | 1.00 | 45.98 | W |
| ATOM | 9104 | OH2 | WAT | W | 1028 | 46.112 | 39.784 | −0.055 | 1.00 | 27.99 | W |
| ATOM | 9105 | OH2 | WAT | W | 1029 | 37.083 | 37.317 | 25.012 | 1.00 | 33.64 | W |
| ATOM | 9106 | OH2 | WAT | W | 1030 | 52.414 | 68.316 | 4.826 | 1.00 | 31.30 | W |
| ATOM | 9107 | OH2 | WAT | W | 1031 | 17.744 | 50.810 | 38.215 | 1.00 | 29.84 | W |
| ATOM | 9108 | OH2 | WAT | W | 1032 | 15.243 | 76.915 | −13.620 | 1.00 | 29.17 | W |
| ATOM | 9109 | OH2 | WAT | W | 1033 | 20.102 | 39.479 | −12.507 | 1.00 | 34.90 | W |
| ATOM | 9110 | OH2 | WAT | W | 1034 | 49.415 | 45.555 | 31.017 | 1.00 | 29.53 | W |
| ATOM | 9111 | OH2 | WAT | W | 1035 | 46.778 | 72.403 | −17.522 | 1.00 | 29.14 | W |
| ATOM | 9112 | OH2 | WAT | W | 1036 | 70.421 | 78.009 | −26.052 | 1.00 | 28.22 | W |
| ATOM | 9113 | OH2 | WAT | W | 1037 | 58.470 | 52.894 | 4.808 | 1.00 | 24.37 | W |
| ATOM | 9114 | OH2 | WAT | W | 1038 | 35.575 | 55.341 | −34.684 | 1.00 | 34.14 | W |
| ATOM | 9115 | OH2 | WAT | W | 1039 | 41.336 | 82.761 | −28.764 | 1.00 | 34.42 | W |
| ATOM | 9116 | OH2 | WAT | W | 1040 | 47.748 | 66.115 | 26.835 | 1.00 | 30.73 | W |
| ATOM | 9117 | OH2 | WAT | W | 1041 | 28.436 | 68.129 | 11.340 | 1.00 | 30.03 | W |
| ATOM | 9118 | OH2 | WAT | W | 1043 | 39.499 | 81.827 | −21.986 | 1.00 | 34.41 | W |
| ATOM | 9119 | OH2 | WAT | W | 1044 | 22.020 | 65.686 | 32.120 | 1.00 | 29.09 | W |
| ATOM | 9120 | OH2 | WAT | W | 1045 | 58.777 | 48.905 | 18.951 | 1.00 | 31.31 | W |
| ATOM | 9121 | OH2 | WAT | W | 1046 | 31.999 | 80.814 | 6.975 | 1.00 | 45.48 | W |
| ATOM | 9122 | OH2 | WAT | W | 1047 | 50.409 | 44.928 | −24.885 | 1.00 | 35.65 | W |
| ATOM | 9123 | OH2 | WAT | W | 1048 | 62.308 | 60.740 | 0.550 | 1.00 | 29.11 | W |
| ATOM | 9124 | OH2 | WAT | W | 1049 | 67.874 | 75.083 | −35.117 | 1.00 | 29.34 | W |
| ATOM | 9125 | OH2 | WAT | W | 1050 | 23.178 | 39.391 | −15.604 | 1.00 | 36.12 | W |
| ATOM | 9126 | OH2 | WAT | W | 1051 | 35.884 | 93.525 | −29.986 | 1.00 | 34.46 | W |
| ATOM | 9127 | OH2 | WAT | W | 1052 | 73.994 | 79.960 | −10.916 | 1.00 | 30.75 | W |
| ATOM | 9128 | OH2 | WAT | W | 1053 | 40.966 | 81.760 | −3.585 | 1.00 | 34.92 | W |
| ATOM | 9129 | OH2 | WAT | W | 1054 | 12.972 | 62.847 | 12.055 | 1.00 | 42.92 | W |
| ATOM | 9130 | OH2 | WAT | W | 1055 | 56.938 | 75.884 | 0.308 | 1.00 | 32.67 | W |
| ATOM | 9131 | OH2 | WAT | W | 1056 | 74.294 | 52.058 | 1.453 | 1.00 | 31.78 | W |
| ATOM | 9132 | OH2 | WAT | W | 1057 | 22.310 | 61.172 | −35.052 | 1.00 | 32.26 | W |
| ATOM | 9133 | OH2 | WAT | W | 1058 | 77.454 | 65.185 | −14.847 | 1.00 | 28.32 | W |
| ATOM | 9134 | OH2 | WAT | W | 1059 | 17.651 | 41.747 | 32.191 | 1.00 | 47.09 | W |
| ATOM | 9135 | OH2 | WAT | W | 1060 | 10.105 | 62.757 | −15.140 | 1.00 | 33.54 | W |
| ATOM | 9136 | OH2 | WAT | W | 1061 | 46.034 | 79.593 | 4.983 | 1.00 | 29.18 | W |
| ATOM | 9137 | OH2 | WAT | W | 1062 | 14.610 | 46.758 | 32.147 | 1.00 | 30.79 | W |
| ATOM | 9138 | OH2 | WAT | W | 1063 | 34.557 | 77.270 | 12.439 | 1.00 | 27.02 | W |
| ATOM | 9139 | OH2 | WAT | W | 1064 | 47.268 | 40.156 | 6.116 | 1.00 | 33.32 | W |
| ATOM | 9140 | OH2 | WAT | W | 1065 | 18.619 | 39.189 | −2.666 | 1.00 | 33.06 | W |
| ATOM | 9141 | OH2 | WAT | W | 1066 | 26.043 | 58.928 | −38.980 | 1.00 | 35.17 | W |
| ATOM | 9142 | OH2 | WAT | W | 1067 | 10.705 | 57.867 | −20.854 | 1.00 | 35.84 | W |
| ATOM | 9143 | OH2 | WAT | W | 1068 | 8.759 | 56.628 | 16.327 | 1.00 | 41.90 | W |
| ATOM | 9144 | OH2 | WAT | W | 1069 | 78.041 | 72.263 | −10.242 | 1.00 | 33.75 | W |
| ATOM | 9145 | OH2 | WAT | W | 1070 | 24.072 | 82.790 | −3.954 | 1.00 | 45.00 | W |
| ATOM | 9146 | OH2 | WAT | W | 1071 | 44.516 | 40.813 | 7.609 | 1.00 | 39.23 | W |
| ATOM | 9147 | OH2 | WAT | W | 1072 | 35.419 | 62.216 | 34.108 | 1.00 | 29.33 | W |
| ATOM | 9148 | OH2 | WAT | W | 1073 | 27.207 | 89.776 | −31.767 | 1.00 | 34.55 | W |
| ATOM | 9149 | OH2 | WAT | W | 1074 | 74.676 | 77.261 | −20.637 | 1.00 | 34.22 | W |
| ATOM | 9150 | OH2 | WAT | W | 1075 | 49.177 | 41.888 | 8.315 | 1.00 | 31.43 | W |
| ATOM | 9151 | OH2 | WAT | W | 1076 | 44.832 | 92.668 | −25.355 | 1.00 | 33.39 | W |
| ATOM | 9152 | OH2 | WAT | W | 1077 | 52.396 | 48.868 | 25.516 | 1.00 | 32.38 | W |
| ATOM | 9153 | OH2 | WAT | W | 1078 | 9.675 | 53.484 | −12.053 | 1.00 | 29.84 | W |
| ATOM | 9154 | OH2 | WAT | W | 1079 | 58.765 | 51.065 | 14.476 | 1.00 | 46.60 | W |
| ATOM | 9155 | OH2 | WAT | W | 1080 | 51.794 | 50.452 | 27.587 | 1.00 | 30.65 | W |
| ATOM | 9156 | OH2 | WAT | W | 1081 | 25.081 | 88.824 | −25.170 | 1.00 | 31.67 | W |
| ATOM | 9157 | OH2 | WAT | W | 1082 | 7.589 | 58.168 | 0.166 | 1.00 | 32.04 | W |
| ATOM | 9158 | OH2 | WAT | W | 1083 | 11.415 | 58.349 | 19.950 | 1.00 | 34.77 | W |
| ATOM | 9159 | OH2 | WAT | W | 1084 | 18.559 | 60.815 | −37.787 | 1.00 | 30.54 | W |
| ATOM | 9160 | OH2 | WAT | W | 1085 | 46.111 | 60.046 | −31.330 | 1.00 | 34.66 | W |
| ATOM | 9161 | OH2 | WAT | W | 1086 | 5.566 | 57.763 | 6.910 | 1.00 | 31.07 | W |
| ATOM | 9162 | OH2 | WAT | W | 1087 | 13.431 | 66.030 | −11.696 | 1.00 | 39.54 | W |
| ATOM | 9163 | OH2 | WAT | W | 1088 | 47.191 | 81.448 | −17.052 | 1.00 | 31.72 | W |
| ATOM | 9164 | C1 | NAG | C | 1 | 58.272 | 44.933 | 12.939 | 1.00 | 54.15 | C |
| ATOM | 9165 | C2 | NAG | C | 1 | 59.491 | 44.597 | 13.810 | 1.00 | 54.12 | C |
| ATOM | 9166 | N2 | NAG | C | 1 | 60.574 | 45.520 | 13.526 | 1.00 | 56.17 | C |
| ATOM | 9167 | C7 | NAG | C | 1 | 60.706 | 46.633 | 14.241 | 1.00 | 68.32 | C |
| ATOM | 9168 | O7 | NAG | C | 1 | 60.206 | 47.706 | 13.905 | 1.00 | 84.10 | C |
| ATOM | 9169 | C8 | NAG | C | 1 | 61.520 | 46.545 | 15.522 | 1.00 | 55.90 | C |
| ATOM | 9170 | C3 | NAG | C | 1 | 59.957 | 43.162 | 13.548 | 1.00 | 55.37 | C |
| ATOM | 9171 | O3 | NAG | C | 1 | 60.989 | 42.822 | 14.463 | 1.00 | 58.48 | C |
| ATOM | 9172 | C4 | NAG | C | 1 | 58.791 | 42.186 | 13.705 | 1.00 | 51.27 | C |
| ATOM | 9173 | O4 | NAG | C | 1 | 59.208 | 40.880 | 13.335 | 1.00 | 55.79 | C |
| ATOM | 9174 | C5 | NAG | C | 1 | 57.623 | 42.627 | 12.819 | 1.00 | 61.43 | C |
| ATOM | 9175 | O5 | NAG | C | 1 | 57.227 | 43.975 | 13.158 | 1.00 | 49.30 | C |
| ATOM | 9176 | C6 | NAG | C | 1 | 56.402 | 41.745 | 12.991 | 1.00 | 77.13 | C |
| ATOM | 9177 | O6 | NAG | C | 1 | 56.268 | 40.837 | 11.908 | 1.00 | 60.25 | C |
| ATOM | 9178 | C | TRS | T | 1 | 31.353 | 66.569 | 7.597 | 1.00 | 15.84 | T |
| ATOM | 9179 | C1 | TRS | T | 1 | 31.240 | 66.938 | 6.107 | 1.00 | 15.64 | T |
| ATOM | 9180 | C2 | TRS | T | 1 | 32.708 | 66.586 | 7.995 | 1.00 | 15.85 | T |
| ATOM | 9181 | C3 | TRS | T | 1 | 30.629 | 65.227 | 7.717 | 1.00 | 16.29 | T |
| ATOM | 9182 | N | TRS | T | 1 | 30.638 | 67.570 | 8.399 | 1.00 | 15.69 | T |
| ATOM | 9183 | O1 | TRS | T | 1 | 31.683 | 68.184 | 5.843 | 1.00 | 15.45 | T |
| ATOM | 9184 | O2 | TRS | T | 1 | 33.643 | 65.910 | 7.226 | 1.00 | 13.34 | T |
| ATOM | 9185 | O3 | TRS | T | 1 | 30.581 | 64.645 | 8.982 | 1.00 | 18.13 | T |
| ATOM | 9186 | C1 | MPD | M | 1 | 14.883 | 61.068 | 10.331 | 1.00 | 17.96 | M |
| ATOM | 9187 | C2 | MPD | M | 1 | 16.351 | 61.254 | 10.649 | 1.00 | 18.97 | M |
| ATOM | 9188 | O2 | MPD | M | 1 | 16.957 | 60.096 | 9.890 | 1.00 | 19.78 | M |
| ATOM | 9189 | CM | MPD | M | 1 | 17.188 | 62.371 | 10.051 | 1.00 | 19.99 | M |
| ATOM | 9190 | C3 | MPD | M | 1 | 16.549 | 61.049 | 12.169 | 1.00 | 18.43 | M |
| ATOM | 9191 | C4 | MPD | M | 1 | 17.848 | 60.686 | 12.721 | 1.00 | 17.69 | M |
| ATOM | 9192 | O4 | MPD | M | 1 | 17.567 | 59.714 | 13.729 | 1.00 | 16.05 | M |
| ATOM | 9193 | C5 | MPD | M | 1 | 18.419 | 61.960 | 13.429 | 1.00 | 17.96 | M |
| ATOM | 9194 | ZN | ZN | Z | 1 | 34.680 | 64.059 | 7.920 | 1.00 | 9.96 | Z |
| END | | | | | | | | | | | |

TABLE 2
Structural coordinates of a Drosophila Golgi α-mannosidase II with swainsonine.

REMARK coordinates from restrained individual B-factor refinement
REMARK refinement resolution: 500.0 − 1.87 A
REMARK starting r = 0.1835 free_r = 0.2089
REMARK final r = 0.1801 free_r = 0.2084
REMARK B rmsd for bonded mainchain atoms = 0.707 target = 1.5
REMARK B rmsd for bonded sidechain atoms = 1.139 target = 2.0
REMARK B rmsd for angle mainchain atoms = 1.167 target = 2.0
REMARK B rmsd for angle sidechain atoms = 1.765 target = 2.5
REMARK wa = 1.14241
REMARK rweight = 0.269445
REMARK target = mlf steps = 30
REMARK sg = P2 (1) 2 (1) 2 (1) a = 68.902 b = 110.015 c = 138.472 alpha = 90
beta = 90 gamma = 90
REMARK parameter file 1 CNS_TOPPAR:protein_rep.param
REMARK parameter file 2 CNS_TOPPAR:water_rep.param
REMARK parameter file 3 CNS_TOPPAR:ion.param
REMARK parameter file 4 swainsonine2.par
REMARK parameter file 5 ../zntrmp/mpd.par
REMARK parameter file 6 cis_peptide.param
REMARK parameter file 7 CNS_TOPPAR:carbohydrate.param
REMARK molecular structure file: swainsoninegen.mtf
REMARK input coordinates: swainsonine_ann_1.pdb
REMARK reflection file = dgm2native_rejmerge.cv
REMARK ncs = none
REMARK B-correction resolution: 6.0 − 1.87
REMARK initial B-factor correction applied to fobs:
REMARK B11 = 0.513 B22 = −0.085 B33 = −0.428
REMARK B12 = 0.000 B13 = 0.000 B23 = 0.000
REMARK B-factor correction applied to coordinate array B: −0.306
REMARK bulk solvent: density level = 0.353213 e/A♯ 3♯, B-factor = 42.0423 A♯ ♯2
REMARK reflections with |Fobs|/sigma_F < 0.0 rejected
REMARK reflections with |Fobs| > 10000 * rms (Fobs) rejected

| | | |
|---|---|---|
| REMARK theoretical total number of refl. in resol. range: | 87643 | (100.0%) |
| REMARK number of unobserved reflections (no entry or \|F\| = 0): | 2814 | (3.2%) |
| REMARK number of reflections rejected: | 0 | (0.0%) |
| REMARK total number of reflections used: | 84829 | (96.8%) |
| REMARK number of reflections in working set: | 80543 | (91.9%) |
| REMARK number of reflections in test set: | 4286 | (4.9%) |

CRYST1 68.902 110.015 138.472 90.00 90.00 90.00 P 21 21 21
REMARK FILENAME = “swainsonine_ann_1bi.pdb”
REMARK DATE:15-Jul-2000 00:41:05 created by user: jvdelsen
REMARK VERSION: 0.9a

| | | | | | | | | | | | |
|---|---|---|---|---|---|---|---|---|---|---|---|
| ATOM | 1 | C | CYS | A | 31 | 41.925 | 37.251 | −18.672 | 1.00 | 24.62 | A |
| ATOM | 1 | C | CYS | A | 31 | 41.925 | 37.251 | −18.672 | 1.00 | 24.62 | A |
| ATOM | 2 | O | CYS | A | 31 | 41.435 | 36.638 | −19.619 | 1.00 | 24.13 | A |
| ATOM | 3 | CB | CYS | A | 31 | 43.816 | 38.560 | −19.547 | 1.00 | 23.99 | A |
| ATOM | 4 | SG | CYS | A | 31 | 45.498 | 39.239 | −19.413 | 1.00 | 23.62 | A |
| ATOM | 5 | N | CYS | A | 31 | 44.191 | 36.197 | −18.869 | 1.00 | 25.37 | A |
| ATOM | 6 | CA | CYS | A | 31 | 43.431 | 37.448 | −18.573 | 1.00 | 24.59 | A |
| ATOM | 7 | N | GLN | A | 32 | 41.195 | 37.782 | −17.695 | 1.00 | 24.51 | A |
| ATOM | 8 | CA | GLN | A | 32 | 39.740 | 37.694 | −17.701 | 1.00 | 24.50 | A |
| ATOM | 9 | CB | GLN | A | 32 | 39.148 | 38.167 | −16.372 | 1.00 | 25.89 | A |
| ATOM | 10 | CG | GLN | A | 32 | 39.164 | 37.148 | −15.257 | 1.00 | 28.92 | A |
| ATOM | 11 | CD | GLN | A | 32 | 38.247 | 37.547 | −14.117 | 1.00 | 30.46 | A |
| ATOM | 12 | OE1 | GLN | A | 32 | 37.028 | 37.625 | −14.286 | 1.00 | 31.32 | A |
| ATOM | 13 | NE2 | GLN | A | 32 | 38.827 | 37.811 | −12.951 | 1.00 | 31.69 | A |
| ATOM | 14 | C | GLN | A | 32 | 39.201 | 38.595 | −18.801 | 1.00 | 23.50 | A |
| ATOM | 15 | O | GLN | A | 32 | 39.787 | 39.632 | −19.112 | 1.00 | 22.91 | A |
| ATOM | 16 | N | ASP | A | 33 | 38.084 | 38.194 | −19.389 | 1.00 | 22.48 | A |
| ATOM | 17 | CA | ASP | A | 33 | 37.455 | 38.986 | −20.432 | 1.00 | 21.66 | A |
| ATOM | 18 | CB | ASP | A | 33 | 36.645 | 38.066 | −21.350 | 1.00 | 21.90 | A |
| ATOM | 19 | CG | ASP | A | 33 | 36.033 | 38.799 | −22.524 | 1.00 | 23.08 | A |
| ATOM | 20 | OD1 | ASP | A | 33 | 35.768 | 38.140 | −23.553 | 1.00 | 22.70 | A |
| ATOM | 21 | OD2 | ASP | A | 33 | 35.804 | 40.023 | −22.415 | 1.00 | 23.45 | A |
| ATOM | 22 | C | ASP | A | 33 | 36.554 | 39.956 | −19.671 | 1.00 | 20.60 | A |
| ATOM | 23 | O | ASP | A | 33 | 35.617 | 39.536 | −18.999 | 1.00 | 21.58 | A |
| ATOM | 24 | N | VAL | A | 34 | 36.846 | 41.250 | −19.759 | 1.00 | 18.58 | A |
| ATOM | 25 | CA | VAL | A | 34 | 36.066 | 42.246 | −19.030 | 1.00 | 16.33 | A |
| ATOM | 26 | CB | VAL | A | 34 | 36.971 | 43.414 | −18.559 | 1.00 | 16.29 | A |
| ATOM | 27 | CG1 | VAL | A | 34 | 38.137 | 42.869 | −17.752 | 1.00 | 16.34 | A |
| ATOM | 28 | CG2 | VAL | A | 34 | 37.493 | 44.200 | −19.760 | 1.00 | 15.50 | A |
| ATOM | 29 | C | VAL | A | 34 | 34.895 | 42.818 | −19.819 | 1.00 | 15.89 | A |
| ATOM | 30 | O | VAL | A | 34 | 34.213 | 43.728 | −19.353 | 1.00 | 14.74 | A |
| ATOM | 31 | N | VAL | A | 35 | 34.641 | 42.268 | −21.001 | 1.00 | 14.60 | A |
| ATOM | 32 | CA | VAL | A | 35 | 33.559 | 42.769 | −21.837 | 1.00 | 15.46 | A |
| ATOM | 33 | CB | VAL | A | 35 | 34.095 | 43.167 | −23.237 | 1.00 | 15.53 | A |
| ATOM | 34 | CG1 | VAL | A | 35 | 32.950 | 43.676 | −24.118 | 1.00 | 15.86 | A |
| ATOM | 35 | CG2 | VAL | A | 35 | 35.188 | 44.216 | −23.098 | 1.00 | 14.85 | A |
| ATOM | 36 | C | VAL | A | 35 | 32.383 | 41.821 | −22.065 | 1.00 | 16.31 | A |
| ATOM | 37 | O | VAL | A | 35 | 31.225 | 42.207 | −21.908 | 1.00 | 15.61 | A |
| ATOM | 38 | N | GLN | A | 36 | 32.692 | 40.579 | −22.418 | 1.00 | 17.91 | A |
| ATOM | 39 | CA | GLN | A | 36 | 31.673 | 39.590 | −22.771 | 1.00 | 19.46 | A |
| ATOM | 40 | CB | GLN | A | 36 | 32.219 | 38.736 | −23.913 | 1.00 | 19.69 | A |
| ATOM | 41 | CG | GLN | A | 36 | 32.976 | 39.558 | −24.943 | 1.00 | 20.71 | A |
| ATOM | 42 | CD | GLN | A | 36 | 33.442 | 38.735 | −26.117 | 1.00 | 21.13 | A |
| ATOM | 43 | OE1 | GLN | A | 36 | 32.667 | 38.436 | −27.022 | 1.00 | 22.57 | A |
| ATOM | 44 | NE2 | GLN | A | 36 | 34.714 | 38.355 | −26.106 | 1.00 | 21.31 | A |
| ATOM | 45 | C | GLN | A | 36 | 31.065 | 38.670 | −21.721 | 1.00 | 20.47 | A |
| ATOM | 46 | O | GLN | A | 36 | 30.117 | 37.948 | −22.023 | 1.00 | 20.77 | A |
| ATOM | 47 | N | ASP | A | 37 | 31.593 | 38.674 | −20.504 | 1.00 | 21.45 | A |
| ATOM | 48 | CA | ASP | A | 37 | 31.051 | 37.811 | −19.459 | 1.00 | 22.08 | A |
| ATOM | 49 | CB | ASP | A | 37 | 32.147 | 36.912 | −18.869 | 1.00 | 23.31 | A |
| ATOM | 50 | CG | ASP | A | 37 | 32.736 | 35.956 | −19.887 | 1.00 | 24.58 | A |
| ATOM | 51 | OD1 | ASP | A | 37 | 31.959 | 35.302 | −20.610 | 1.00 | 25.48 | A |
| ATOM | 52 | OD2 | ASP | A | 37 | 33.979 | 35.851 | −19.955 | 1.00 | 25.49 | A |
| ATOM | 53 | C | ASP | A | 37 | 30.416 | 38.614 | −18.330 | 1.00 | 21.91 | A |
| ATOM | 54 | O | ASP | A | 37 | 31.120 | 39.195 | −17.506 | 1.00 | 22.46 | A |
| ATOM | 55 | N | VAL | A | 38 | 29.088 | 38.635 | −18.292 | 1.00 | 21.35 | A |
| ATOM | 56 | CA | VAL | A | 38 | 28.363 | 39.354 | −17.249 | 1.00 | 21.02 | A |
| ATOM | 57 | CB | VAL | A | 38 | 26.860 | 39.448 | −17.578 | 1.00 | 21.79 | A |
| ATOM | 58 | CG1 | VAL | A | 38 | 26.122 | 40.191 | −16.466 | 1.00 | 21.12 | A |
| ATOM | 59 | CG2 | VAL | A | 38 | 26.669 | 40.156 | −18.916 | 1.00 | 21.34 | A |
| ATOM | 60 | C | VAL | A | 38 | 28.523 | 38.633 | −15.912 | 1.00 | 21.06 | A |
| ATOM | 61 | O | VAL | A | 38 | 28.042 | 37.511 | −15.734 | 1.00 | 20.07 | A |
| ATOM | 62 | N | PRO | A | 39 | 29.209 | 39.268 | −14.951 | 1.00 | 20.56 | A |
| ATOM | 63 | CD | PRO | A | 39 | 29.929 | 40.552 | −15.034 | 1.00 | 20.77 | A |
| ATOM | 64 | CA | PRO | A | 39 | 29.407 | 38.646 | −13.640 | 1.00 | 20.50 | A |
| ATOM | 65 | CB | PRO | A | 39 | 30.153 | 39.725 | −12.858 | 1.00 | 20.74 | A |
| ATOM | 66 | CG | PRO | A | 39 | 30.950 | 40.416 | −13.922 | 1.00 | 19.82 | A |
| ATOM | 67 | C | PRO | A | 39 | 28.100 | 38.255 | −12.962 | 1.00 | 20.83 | A |
| ATOM | 68 | O | PRO | A | 39 | 27.092 | 38.957 | −13.066 | 1.00 | 20.74 | A |
| ATOM | 69 | N | ASN | A | 40 | 28.114 | 37.120 | −12.273 | 1.00 | 21.49 | A |
| ATOM | 70 | CA | ASN | A | 40 | 26.931 | 36.674 | −11.555 | 1.00 | 21.21 | A |
| ATOM | 71 | CB | ASN | A | 40 | 26.737 | 35.166 | −11.702 | 1.00 | 23.69 | A |
| ATOM | 72 | CG | ASN | A | 40 | 25.572 | 34.653 | −10.881 | 1.00 | 25.91 | A |
| ATOM | 73 | OD1 | ASN | A | 40 | 24.457 | 35.173 | −10.971 | 1.00 | 28.05 | A |
| ATOM | 74 | ND2 | ASN | A | 40 | 25.820 | 33.629 | −10.073 | 1.00 | 27.66 | A |
| ATOM | 75 | C | ASN | A | 40 | 27.119 | 37.023 | −10.085 | 1.00 | 20.01 | A |
| ATOM | 76 | O | ASN | A | 40 | 27.906 | 36.386 | −9.384 | 1.00 | 19.40 | A |
| ATOM | 77 | N | VAL | A | 41 | 26.405 | 38.040 | −9.620 | 1.00 | 18.76 | A |
| ATOM | 78 | CA | VAL | A | 41 | 26.515 | 38.459 | −8.227 | 1.00 | 17.66 | A |
| ATOM | 79 | CB | VAL | A | 41 | 27.126 | 39.873 | −8.117 | 1.00 | 17.28 | A |
| ATOM | 80 | CG1 | VAL | A | 41 | 28.559 | 39.861 | −8.627 | 1.00 | 17.55 | A |
| ATOM | 81 | CG2 | VAL | A | 41 | 26.291 | 40.860 | −8.919 | 1.00 | 17.64 | A |
| ATOM | 82 | C | VAL | A | 41 | 25.154 | 38.454 | −7.545 | 1.00 | 16.81 | A |
| ATOM | 83 | O | VAL | A | 41 | 24.118 | 38.550 | −8.202 | 1.00 | 16.76 | A |
| ATOM | 84 | N | ASP | A | 42 | 25.161 | 38.333 | −6.223 | 1.00 | 16.37 | A |
| ATOM | 85 | CA | ASP | A | 42 | 23.922 | 38.315 | −5.459 | 1.00 | 16.16 | A |
| ATOM | 86 | CB | ASP | A | 42 | 24.204 | 37.903 | −4.012 | 1.00 | 17.05 | A |
| ATOM | 87 | CG | ASP | A | 42 | 24.742 | 36.488 | −3.906 | 1.00 | 17.52 | A |
| ATOM | 88 | OD1 | ASP | A | 42 | 24.073 | 35.565 | −4.414 | 1.00 | 18.85 | A |
| ATOM | 89 | OD2 | ASP | A | 42 | 25.824 | 36.297 | −3.316 | 1.00 | 17.26 | A |
| ATOM | 90 | C | ASP | A | 42 | 23.255 | 39.681 | −5.484 | 1.00 | 16.06 | A |
| ATOM | 91 | O | ASP | A | 42 | 22.029 | 39.786 | −5.518 | 1.00 | 16.22 | A |
| ATOM | 92 | N | VAL | A | 43 | 24.071 | 40.729 | −5.459 | 1.00 | 15.36 | A |
| ATOM | 93 | CA | VAL | A | 43 | 23.563 | 42.092 | −5.486 | 1.00 | 15.17 | A |
| ATOM | 94 | CB | VAL | A | 43 | 23.726 | 42.788 | −4.118 | 1.00 | 15.84 | A |
| ATOM | 95 | CG1 | VAL | A | 43 | 23.132 | 44.194 | −4.175 | 1.00 | 15.15 | A |
| ATOM | 96 | CG2 | VAL | A | 43 | 23.059 | 41.968 | −3.024 | 1.00 | 16.17 | A |
| ATOM | 97 | C | VAL | A | 43 | 24.315 | 42.920 | −6.521 | 1.00 | 14.54 | A |
| ATOM | 98 | O | VAL | A | 43 | 25.540 | 43.030 | −6.470 | 1.00 | 14.32 | A |
| ATOM | 99 | N | GLN | A | 44 | 23.578 | 43.480 | −7.472 | 1.00 | 13.35 | A |
| ATOM | 100 | CA | GLN | A | 44 | 24.173 | 44.329 | −8.497 | 1.00 | 13.57 | A |
| ATOM | 101 | CB | GLN | A | 44 | 23.958 | 43.724 | −9.889 | 1.00 | 13.33 | A |
| ATOM | 102 | CG | GLN | A | 44 | 25.023 | 44.140 | −10.898 | 1.00 | 12.33 | A |
| ATOM | 103 | CD | GLN | A | 44 | 25.129 | 45.649 | −11.016 | 1.00 | 12.76 | A |
| ATOM | 104 | OE1 | GLN | A | 44 | 24.145 | 46.325 | −11.302 | 1.00 | 12.19 | A |
| ATOM | 105 | NE2 | GLN | A | 44 | 26.325 | 46.184 | −10.792 | 1.00 | 11.46 | A |
| ATOM | 106 | C | GLN | A | 44 | 23.413 | 45.642 | −8.323 | 1.00 | 13.24 | A |
| ATOM | 107 | O | GLN | A | 44 | 22.210 | 45.717 | −8.576 | 1.00 | 13.21 | A |
| ATOM | 108 | N | MET | A | 45 | 24.118 | 46.675 | −7.874 | 1.00 | 13.15 | A |
| ATOM | 109 | CA | MET | A | 45 | 23.485 | 47.952 | −7.577 | 1.00 | 13.00 | A |
| ATOM | 110 | CB | MET | A | 45 | 24.536 | 48.945 | −7.075 | 1.00 | 13.03 | A |
| ATOM | 111 | CG | MET | A | 45 | 25.143 | 48.547 | −5.728 | 1.00 | 13.17 | A |
| ATOM | 112 | SD | MET | A | 45 | 23.891 | 48.155 | −4.467 | 1.00 | 15.24 | A |
| ATOM | 113 | CE | MET | A | 45 | 23.318 | 49.806 | −4.040 | 1.00 | 14.85 | A |
| ATOM | 114 | C | MET | A | 45 | 22.593 | 48.603 | −8.632 | 1.00 | 12.70 | A |
| ATOM | 115 | O | MET | A | 45 | 21.596 | 49.231 | −8.278 | 1.00 | 12.29 | A |
| ATOM | 116 | N | LEU | A | 46 | 22.929 | 48.469 | −9.911 | 1.00 | 12.61 | A |
| ATOM | 117 | CA | LEU | A | 46 | 22.087 | 49.066 | −10.947 | 1.00 | 13.13 | A |
| ATOM | 118 | CB | LEU | A | 46 | 22.778 | 49.012 | −12.316 | 1.00 | 12.68 | A |
| ATOM | 119 | CG | LEU | A | 46 | 22.021 | 49.708 | −13.456 | 1.00 | 11.38 | A |
| ATOM | 120 | CD1 | LEU | A | 46 | 21.998 | 51.215 | −13.217 | 1.00 | 12.44 | A |
| ATOM | 121 | CD2 | LEU | A | 46 | 22.686 | 49.394 | −14.788 | 1.00 | 12.03 | A |
| ATOM | 122 | C | LEU | A | 46 | 20.770 | 48.290 | −11.003 | 1.00 | 14.23 | A |
| ATOM | 123 | O | LEU | A | 46 | 19.687 | 48.876 | −11.131 | 1.00 | 14.08 | A |
| ATOM | 124 | N | GLU | A | 47 | 20.867 | 46.966 | −10.895 | 1.00 | 15.02 | A |
| ATOM | 125 | CA | GLU | A | 47 | 19.682 | 46.114 | −10.929 | 1.00 | 16.69 | A |
| ATOM | 126 | CB | GLU | A | 47 | 20.087 | 44.635 | −10.951 | 1.00 | 17.61 | A |
| ATOM | 127 | CG | GLU | A | 47 | 18.929 | 43.666 | −11.196 | 1.00 | 20.72 | A |
| ATOM | 128 | CD | GLU | A | 47 | 18.124 | 43.357 | −9.947 | 1.00 | 22.35 | A |
| ATOM | 129 | OE1 | GLU | A | 47 | 17.009 | 42.805 | −10.080 | 1.00 | 23.46 | A |
| ATOM | 130 | OE2 | GLU | A | 47 | 18.601 | 43.648 | −8.831 | 1.00 | 23.19 | A |
| ATOM | 131 | C | GLU | A | 47 | 18.824 | 46.414 | −9.705 | 1.00 | 16.45 | A |
| ATOM | 132 | O | GLU | A | 47 | 17.609 | 46.569 | −9.812 | 1.00 | 17.05 | A |
| ATOM | 133 | N | LEU | A | 48 | 19.465 | 46.511 | −8.545 | 1.00 | 16.44 | A |
| ATOM | 134 | CA | LEU | A | 48 | 18.755 | 46.809 | −7.304 | 1.00 | 16.52 | A |
| ATOM | 135 | CB | LEU | A | 48 | 19.737 | 46.869 | −6.128 | 1.00 | 16.70 | A |
| ATOM | 136 | CG | LEU | A | 48 | 19.127 | 47.139 | −4.748 | 1.00 | 17.63 | A |
| ATOM | 137 | CD1 | LEU | A | 48 | 18.115 | 46.053 | −4.415 | 1.00 | 19.01 | A |
| ATOM | 138 | CD2 | LEU | A | 48 | 20.225 | 47.188 | −3.695 | 1.00 | 18.67 | A |
| ATOM | 139 | C | LEU | A | 48 | 18.019 | 48.141 | −7.420 | 1.00 | 16.10 | A |
| ATOM | 140 | O | LEU | A | 48 | 16.859 | 48.254 | −7.029 | 1.00 | 16.36 | A |
| ATOM | 141 | N | TYR | A | 49 | 18.694 | 49.145 | −7.968 | 1.00 | 15.59 | A |
| ATOM | 142 | CA | TYR | A | 49 | 18.093 | 50.466 | −8.127 | 1.00 | 15.59 | A |
| ATOM | 143 | CB | TYR | A | 49 | 19.121 | 51.445 | −8.712 | 1.00 | 14.86 | A |
| ATOM | 144 | CG | TYR | A | 49 | 19.675 | 52.401 | −7.678 | 1.00 | 14.18 | A |
| ATOM | 145 | CD1 | TYR | A | 49 | 20.150 | 51.929 | −6.451 | 1.00 | 13.14 | A |
| ATOM | 146 | CE1 | TYR | A | 49 | 20.600 | 52.807 | −5.464 | 1.00 | 13.25 | A |
| ATOM | 147 | CD2 | TYR | A | 49 | 19.672 | 53.779 | −7.899 | 1.00 | 13.52 | A |
| ATOM | 148 | CE2 | TYR | A | 49 | 20.120 | 54.666 | −6.920 | 1.00 | 13.75 | A |
| ATOM | 149 | CZ | TYR | A | 49 | 20.578 | 54.175 | −5.706 | 1.00 | 13.51 | A |
| ATOM | 150 | OH | TYR | A | 49 | 20.979 | 55.051 | −4.723 | 1.00 | 13.85 | A |
| ATOM | 151 | C | TYR | A | 49 | 16.850 | 50.415 | −9.009 | 1.00 | 16.60 | A |
| ATOM | 152 | O | TYR | A | 49 | 15.879 | 51.136 | −8.779 | 1.00 | 15.53 | A |
| ATOM | 153 | N | ASP | A | 50 | 16.883 | 49.547 | −10.012 | 1.00 | 17.82 | A |
| ATOM | 154 | CA | ASP | A | 50 | 15.764 | 49.399 | −10.931 | 1.00 | 20.30 | A |
| ATOM | 155 | CB | ASP | A | 50 | 16.153 | 48.440 | −12.061 | 1.00 | 21.45 | A |
| ATOM | 156 | CG | ASP | A | 50 | 15.329 | 48.645 | −13.318 | 1.00 | 23.77 | A |
| ATOM | 157 | OD1 | ASP | A | 50 | 15.403 | 47.778 | −14.215 | 1.00 | 24.48 | A |
| ATOM | 158 | OD2 | ASP | A | 50 | 14.626 | 49.674 | −13.418 | 1.00 | 23.86 | A |
| ATOM | 159 | C | ASP | A | 50 | 14.526 | 48.868 | −10.198 | 1.00 | 21.12 | A |
| ATOM | 160 | O | ASP | A | 50 | 13.403 | 49.294 | −10.476 | 1.00 | 20.00 | A |
| ATOM | 161 | N | ARG | A | 51 | 14.741 | 47.951 | −9.256 | 1.00 | 22.14 | A |
| ATOM | 162 | CA | ARG | A | 51 | 13.651 | 47.336 | −8.494 | 1.00 | 23.40 | A |
| ATOM | 163 | CB | ARG | A | 51 | 14.044 | 45.918 | −8.059 | 1.00 | 25.60 | A |
| ATOM | 164 | CG | ARG | A | 51 | 14.163 | 44.925 | −9.192 | 1.00 | 28.61 | A |
| ATOM | 165 | CD | ARG | A | 51 | 14.338 | 43.491 | −8.689 | 1.00 | 31.12 | A |
| ATOM | 166 | NE | ARG | A | 51 | 15.625 | 43.263 | −8.034 | 1.00 | 33.00 | A |
| ATOM | 167 | CZ | ARG | A | 51 | 15.868 | 43.467 | −6.743 | 1.00 | 33.65 | A |
| ATOM | 168 | NH1 | ARG | A | 51 | 14.908 | 43.906 | −5.940 | 1.00 | 34.72 | A |
| ATOM | 169 | NH2 | ARG | A | 51 | 17.077 | 43.228 | −6.253 | 1.00 | 33.58 | A |
| ATOM | 170 | C | ARG | A | 51 | 13.156 | 48.096 | −7.262 | 1.00 | 23.04 | A |
| ATOM | 171 | O | ARG | A | 51 | 11.979 | 48.014 | −6.921 | 1.00 | 23.00 | A |
| ATOM | 172 | N | MET | A | 52 | 14.047 | 48.820 | −6.591 | 1.00 | 22.18 | A |
| ATOM | 173 | CA | MET | A | 52 | 13.680 | 49.564 | −5.385 | 1.00 | 22.46 | A |
| ATOM | 174 | CB | MET | A | 52 | 14.924 | 50.192 | −4.757 | 1.00 | 22.88 | A |
| ATOM | 175 | CG | MET | A | 52 | 15.886 | 49.195 | −4.152 | 1.00 | 24.34 | A |
| ATOM | 176 | SD | MET | A | 52 | 17.406 | 50.015 | −3.629 | 1.00 | 25.98 | A |
| ATOM | 177 | CE | MET | A | 52 | 16.778 | 51.075 | −2.331 | 1.00 | 26.01 | A |
| ATOM | 178 | C | MET | A | 52 | 12.642 | 50.652 | −5.617 | 1.00 | 21.82 | A |
| ATOM | 179 | O | MET | A | 52 | 12.606 | 51.271 | −6.681 | 1.00 | 21.80 | A |
| ATOM | 180 | N | SER | A | 53 | 11.810 | 50.894 | −4.606 | 1.00 | 21.18 | A |
| ATOM | 181 | CA | SER | A | 53 | 10.762 | 51.908 | −4.696 | 1.00 | 21.22 | A |
| ATOM | 182 | CB | SER | A | 53 | 9.477 | 51.386 | −4.048 | 1.00 | 21.76 | A |
| ATOM | 183 | OG | SER | A | 53 | 8.985 | 50.253 | −4.745 | 1.00 | 23.57 | A |
| ATOM | 184 | C | SER | A | 53 | 11.156 | 53.238 | −4.055 | 1.00 | 20.40 | A |
| ATOM | 185 | O | SER | A | 53 | 10.531 | 54.267 | −4.311 | 1.00 | 20.31 | A |
| ATOM | 186 | N | PHE | A | 54 | 12.185 | 53.204 | −3.214 | 1.00 | 19.97 | A |
| ATOM | 187 | CA | PHE | A | 54 | 12.686 | 54.399 | −2.538 | 1.00 | 19.35 | A |
| ATOM | 188 | CB | PHE | A | 54 | 13.354 | 55.343 | −3.545 | 1.00 | 18.86 | A |
| ATOM | 189 | CG | PHE | A | 54 | 14.600 | 54.784 | −4.174 | 1.00 | 17.89 | A |
| ATOM | 190 | CD1 | PHE | A | 54 | 14.522 | 53.912 | −5.256 | 1.00 | 17.68 | A |
| ATOM | 191 | CD2 | PHE | A | 54 | 15.852 | 55.127 | −3.677 | 1.00 | 17.36 | A |
| ATOM | 192 | CE1 | PHE | A | 54 | 15.674 | 53.387 | −5.837 | 1.00 | 17.70 | A |
| ATOM | 193 | CE2 | PHE | A | 54 | 17.015 | 54.609 | −4.247 | 1.00 | 17.77 | A |
| ATOM | 194 | CZ | PHE | A | 54 | 16.929 | 53.736 | −5.329 | 1.00 | 18.12 | A |
| ATOM | 195 | C | PHE | A | 54 | 11.644 | 55.188 | −1.747 | 1.00 | 19.63 | A |
| ATOM | 196 | O | PHE | A | 54 | 11.729 | 56.414 | −1.660 | 1.00 | 18.67 | A |
| ATOM | 197 | N | LYS | A | 55 | 10.664 | 54.504 | −1.165 | 1.00 | 19.83 | A |
| ATOM | 198 | CA | LYS | A | 55 | 9.653 | 55.211 | −0.387 | 1.00 | 20.73 | A |
| ATOM | 199 | CB | LYS | A | 55 | 8.437 | 54.313 | −0.134 | 1.00 | 20.77 | A |
| ATOM | 200 | CG | LYS | A | 55 | 7.748 | 53.848 | −1.403 | 1.00 | 20.88 | A |
| ATOM | 201 | CD | LYS | A | 55 | 7.336 | 55.018 | −2.284 | 1.00 | 21.60 | A |
| ATOM | 202 | CE | LYS | A | 55 | 6.699 | 54.527 | −3.578 | 1.00 | 21.98 | A |
| ATOM | 203 | NZ | LYS | A | 55 | 6.215 | 55.652 | −4.429 | 1.00 | 21.47 | A |
| ATOM | 204 | C | LYS | A | 55 | 10.265 | 55.652 | 0.936 | 1.00 | 20.90 | A |
| ATOM | 205 | O | LYS | A | 55 | 10.925 | 54.870 | 1.615 | 1.00 | 21.59 | A |
| ATOM | 206 | N | ASP | A | 56 | 10.045 | 56.912 | 1.291 | 1.00 | 21.05 | A |
| ATOM | 207 | CA | ASP | A | 56 | 10.582 | 57.478 | 2.522 | 1.00 | 21.70 | A |
| ATOM | 208 | CB | ASP | A | 56 | 11.094 | 58.897 | 2.234 | 1.00 | 20.78 | A |
| ATOM | 209 | CG | ASP | A | 56 | 11.697 | 59.567 | 3.450 | 1.00 | 20.27 | A |
| ATOM | 210 | OD1 | ASP | A | 56 | 12.238 | 58.861 | 4.324 | 1.00 | 20.21 | A |
| ATOM | 211 | OD2 | ASP | A | 56 | 11.642 | 60.812 | 3.523 | 1.00 | 20.23 | A |
| ATOM | 212 | C | ASP | A | 56 | 9.520 | 57.493 | 3.622 | 1.00 | 22.51 | A |
| ATOM | 213 | O | ASP | A | 56 | 8.954 | 58.536 | 3.939 | 1.00 | 23.55 | A |
| ATOM | 214 | N | ILE | A | 57 | 9.251 | 56.331 | 4.207 | 1.00 | 23.05 | A |
| ATOM | 215 | CA | ILE | A | 57 | 8.245 | 56.249 | 5.259 | 1.00 | 23.95 | A |
| ATOM | 216 | CB | ILE | A | 57 | 7.368 | 54.981 | 5.114 | 1.00 | 25.04 | A |
| ATOM | 217 | CG2 | ILE | A | 57 | 6.858 | 54.859 | 3.681 | 1.00 | 24.66 | A |
| ATOM | 218 | CG1 | ILE | A | 57 | 8.172 | 53.737 | 5.485 | 1.00 | 25.72 | A |
| ATOM | 219 | CD1 | ILE | A | 57 | 7.335 | 52.480 | 5.564 | 1.00 | 27.21 | A |
| ATOM | 220 | C | ILE | A | 57 | 8.869 | 56.250 | 6.647 | 1.00 | 23.45 | A |
| ATOM | 221 | O | ILE | A | 57 | 10.022 | 55.859 | 6.824 | 1.00 | 23.83 | A |
| ATOM | 222 | N | ASP | A | 58 | 8.091 | 56.698 | 7.627 | 1.00 | 23.42 | A |
| ATOM | 223 | CA | ASP | A | 58 | 8.528 | 56.758 | 9.017 | 1.00 | 23.11 | A |
| ATOM | 224 | CB | ASP | A | 58 | 7.566 | 57.641 | 9.815 | 1.00 | 24.05 | A |
| ATOM | 225 | CG | ASP | A | 58 | 7.986 | 57.817 | 11.264 | 1.00 | 24.91 | A |
| ATOM | 226 | OD1 | ASP | A | 58 | 7.391 | 58.677 | 11.948 | 1.00 | 26.86 | A |
| ATOM | 227 | OD2 | ASP | A | 58 | 8.898 | 57.102 | 11.725 | 1.00 | 24.36 | A |
| ATOM | 228 | C | ASP | A | 58 | 8.552 | 55.347 | 9.599 | 1.00 | 22.47 | A |
| ATOM | 229 | O | ASP | A | 58 | 7.503 | 54.736 | 9.798 | 1.00 | 22.40 | A |
| ATOM | 230 | N | GLY | A | 59 | 9.749 | 54.835 | 9.871 | 1.00 | 21.17 | A |
| ATOM | 231 | CA | GLY | A | 59 | 9.870 | 53.491 | 10.411 | 1.00 | 20.10 | A |
| ATOM | 232 | C | GLY | A | 59 | 9.836 | 53.398 | 11.926 | 1.00 | 18.92 | A |
| ATOM | 233 | O | GLY | A | 59 | 10.040 | 52.321 | 12.486 | 1.00 | 18.96 | A |
| ATOM | 234 | N | GLY | A | 60 | 9.576 | 54.518 | 12.592 | 1.00 | 18.14 | A |
| ATOM | 235 | CA | GLY | A | 60 | 9.529 | 54.523 | 14.045 | 1.00 | 18.27 | A |
| ATOM | 236 | C | GLY | A | 60 | 10.796 | 55.130 | 14.620 | 1.00 | 17.46 | A |
| ATOM | 237 | O | GLY | A | 60 | 11.352 | 56.062 | 14.038 | 1.00 | 17.61 | A |
| ATOM | 238 | N | VAL | A | 61 | 11.264 | 54.612 | 15.752 | 1.00 | 17.48 | A |
| ATOM | 239 | CA | VAL | A | 61 | 12.484 | 55.146 | 16.349 | 1.00 | 16.59 | A |
| ATOM | 240 | CB | VAL | A | 61 | 12.865 | 54.402 | 17.653 | 1.00 | 16.73 | A |
| ATOM | 241 | CG1 | VAL | A | 61 | 11.824 | 54.706 | 18.728 | 1.00 | 16.60 | A |
| ATOM | 242 | CG2 | VAL | A | 61 | 12.957 | 52.904 | 17.413 | 1.00 | 16.85 | A |
| ATOM | 243 | C | VAL | A | 61 | 13.613 | 55.075 | 15.321 | 1.00 | 16.90 | A |
| ATOM | 244 | O | VAL | A | 61 | 14.443 | 55.981 | 15.244 | 1.00 | 16.19 | A |
| ATOM | 245 | N | TRP | A | 62 | 13.651 | 53.998 | 14.537 | 1.00 | 16.72 | A |
| ATOM | 246 | CA | TRP | A | 62 | 14.641 | 53.903 | 13.470 | 1.00 | 17.14 | A |
| ATOM | 247 | CB | TRP | A | 62 | 15.017 | 52.448 | 13.160 | 1.00 | 16.77 | A |
| ATOM | 248 | CG | TRP | A | 62 | 15.981 | 52.323 | 11.999 | 1.00 | 16.61 | A |
| ATOM | 249 | CD2 | TRP | A | 62 | 16.334 | 51.125 | 11.291 | 1.00 | 16.64 | A |
| ATOM | 250 | CE2 | TRP | A | 62 | 17.238 | 51.495 | 10.266 | 1.00 | 16.47 | A |
| ATOM | 251 | CE3 | TRP | A | 62 | 15.974 | 49.776 | 11.421 | 1.00 | 16.05 | A |
| ATOM | 252 | CD1 | TRP | A | 62 | 16.671 | 53.339 | 11.393 | 1.00 | 16.29 | A |
| ATOM | 253 | NE1 | TRP | A | 62 | 17.424 | 52.850 | 10.351 | 1.00 | 16.78 | A |
| ATOM | 254 | CZ2 | TRP | A | 62 | 17.786 | 50.565 | 9.376 | 1.00 | 15.87 | A |
| ATOM | 255 | CZ3 | TRP | A | 62 | 16.518 | 48.849 | 10.537 | 1.00 | 15.80 | A |
| ATOM | 256 | CH2 | TRP | A | 62 | 17.416 | 49.249 | 9.525 | 1.00 | 16.42 | A |
| ATOM | 257 | C | TRP | A | 62 | 13.854 | 54.516 | 12.319 | 1.00 | 17.81 | A |
| ATOM | 258 | O | TRP | A | 62 | 13.199 | 53.816 | 11.542 | 1.00 | 18.20 | A |
| ATOM | 259 | N | LYS | A | 63 | 13.904 | 55.841 | 12.241 | 1.00 | 18.39 | A |
| ATOM | 260 | CA | LYS | A | 63 | 13.159 | 56.598 | 11.243 | 1.00 | 18.98 | A |
| ATOM | 261 | CB | LYS | A | 63 | 13.590 | 58.066 | 11.290 | 1.00 | 20.04 | A |
| ATOM | 262 | CG | LYS | A | 63 | 13.128 | 58.814 | 12.549 | 1.00 | 22.71 | A |
| ATOM | 263 | CD | LYS | A | 63 | 11.608 | 58.979 | 12.573 | 1.00 | 25.03 | A |
| ATOM | 264 | CE | LYS | A | 63 | 11.129 | 59.810 | 13.761 | 1.00 | 26.33 | A |
| ATOM | 265 | NZ | LYS | A | 63 | 11.459 | 59.217 | 15.099 | 1.00 | 28.40 | A |
| ATOM | 266 | C | LYS | A | 63 | 13.180 | 56.108 | 9.800 | 1.00 | 18.64 | A |
| ATOM | 267 | O | LYS | A | 63 | 12.175 | 56.225 | 9.090 | 1.00 | 18.80 | A |
| ATOM | 268 | N | GLN | A | 64 | 14.302 | 55.557 | 9.358 | 1.00 | 17.70 | A |
| ATOM | 269 | CA | GLN | A | 64 | 14.400 | 55.102 | 7.976 | 1.00 | 17.10 | A |
| ATOM | 270 | CB | GLN | A | 64 | 15.610 | 55.768 | 7.320 | 1.00 | 16.66 | A |
| ATOM | 271 | CG | GLN | A | 64 | 15.510 | 57.285 | 7.347 | 1.00 | 16.52 | A |
| ATOM | 272 | CD | GLN | A | 64 | 16.850 | 57.969 | 7.152 | 1.00 | 16.43 | A |
| ATOM | 273 | OE1 | GLN | A | 64 | 17.818 | 57.674 | 7.859 | 1.00 | 16.22 | A |
| ATOM | 274 | NE2 | GLN | A | 64 | 16.910 | 58.894 | 6.198 | 1.00 | 15.17 | A |
| ATOM | 275 | C | GLN | A | 64 | 14.461 | 53.586 | 7.817 | 1.00 | 16.51 | A |
| ATOM | 276 | O | GLN | A | 64 | 14.839 | 53.073 | 6.759 | 1.00 | 16.20 | A |
| ATOM | 277 | N | GLY | A | 65 | 14.070 | 52.880 | 8.873 | 1.00 | 16.63 | A |
| ATOM | 278 | CA | GLY | A | 65 | 14.060 | 51.429 | 8.844 | 1.00 | 17.03 | A |
| ATOM | 279 | C | GLY | A | 65 | 12.713 | 50.881 | 9.284 | 1.00 | 17.98 | A |
| ATOM | 280 | O | GLY | A | 65 | 11.680 | 51.204 | 8.692 | 1.00 | 17.73 | A |
| ATOM | 281 | N | TRP | A | 66 | 12.722 | 50.052 | 10.324 | 1.00 | 18.46 | A |
| ATOM | 282 | CA | TRP | A | 66 | 11.495 | 49.454 | 10.852 | 1.00 | 19.30 | A |
| ATOM | 283 | CB | TRP | A | 66 | 11.101 | 48.231 | 10.012 | 1.00 | 18.93 | A |
| ATOM | 284 | CG | TRP | A | 66 | 12.024 | 47.045 | 10.179 | 1.00 | 18.71 | A |
| ATOM | 285 | CD2 | TRP | A | 66 | 13.222 | 46.776 | 9.440 | 1.00 | 18.40 | A |
| ATOM | 286 | CE2 | TRP | A | 66 | 13.768 | 45.575 | 9.950 | 1.00 | 18.17 | A |
| ATOM | 287 | CE3 | TRP | A | 66 | 13.890 | 47.435 | 8.396 | 1.00 | 18.28 | A |
| ATOM | 288 | CD1 | TRP | A | 66 | 11.897 | 46.026 | 11.081 | 1.00 | 18.40 | A |
| ATOM | 289 | NE1 | TRP | A | 66 | 12.938 | 45.139 | 10.949 | 1.00 | 18.80 | A |
| ATOM | 290 | CZ2 | TRP | A | 66 | 14.952 | 45.015 | 9.452 | 1.00 | 18.67 | A |
| ATOM | 291 | CZ3 | TRP | A | 66 | 15.067 | 46.881 | 7.901 | 1.00 | 18.13 | A |
| ATOM | 292 | CH2 | TRP | A | 66 | 15.587 | 45.681 | 8.431 | 1.00 | 18.63 | A |
| ATOM | 293 | C | TRP | A | 66 | 11.768 | 49.028 | 12.289 | 1.00 | 19.82 | A |
| ATOM | 294 | O | TRP | A | 66 | 12.906 | 49.117 | 12.751 | 1.00 | 19.90 | A |
| ATOM | 295 | N | ASN | A | 67 | 10.735 | 48.581 | 13.000 | 1.00 | 20.61 | A |
| ATOM | 296 | CA | ASN | A | 67 | 10.916 | 48.133 | 14.380 | 1.00 | 21.14 | A |
| ATOM | 297 | CB | ASN | A | 67 | 9.580 | 48.089 | 15.132 | 1.00 | 22.87 | A |
| ATOM | 298 | CG | ASN | A | 67 | 9.005 | 49.469 | 15.382 | 1.00 | 23.90 | A |
| ATOM | 299 | OD1 | ASN | A | 67 | 9.737 | 50.419 | 15.660 | 1.00 | 24.85 | A |
| ATOM | 300 | ND2 | ASN | A | 67 | 7.684 | 49.580 | 15.308 | 1.00 | 24.85 | A |
| ATOM | 301 | C | ASN | A | 67 | 11.534 | 46.742 | 14.365 | 1.00 | 21.15 | A |
| ATOM | 302 | O | ASN | A | 67 | 10.859 | 45.751 | 14.076 | 1.00 | 20.49 | A |
| ATOM | 303 | N | ILE | A | 68 | 12.822 | 46.669 | 14.680 | 1.00 | 20.89 | A |
| ATOM | 304 | CA | ILE | A | 68 | 13.524 | 45.395 | 14.676 | 1.00 | 20.74 | A |
| ATOM | 305 | CB | ILE | A | 68 | 15.047 | 45.602 | 14.791 | 1.00 | 20.37 | A |
| ATOM | 306 | CG2 | ILE | A | 68 | 15.754 | 44.256 | 14.762 | 1.00 | 20.22 | A |
| ATOM | 307 | CG1 | ILE | A | 68 | 15.541 | 46.489 | 13.643 | 1.00 | 19.43 | A |
| ATOM | 308 | CD1 | ILE | A | 68 | 16.996 | 46.907 | 13.771 | 1.00 | 18.98 | A |
| ATOM | 309 | C | ILE | A | 68 | 13.074 | 44.489 | 15.816 | 1.00 | 21.81 | A |
| ATOM | 310 | O | ILE | A | 68 | 12.958 | 44.923 | 16.961 | 1.00 | 21.06 | A |
| ATOM | 311 | N | LYS | A | 69 | 12.820 | 43.227 | 15.486 | 1.00 | 22.54 | A |
| ATOM | 312 | CA | LYS | A | 69 | 12.402 | 42.240 | 16.472 | 1.00 | 23.75 | A |
| ATOM | 313 | CB | LYS | A | 69 | 10.990 | 41.731 | 16.154 | 1.00 | 25.70 | A |
| ATOM | 314 | CG | LYS | A | 69 | 9.910 | 42.796 | 16.310 | 1.00 | 28.27 | A |
| ATOM | 315 | CD | LYS | A | 69 | 8.529 | 42.293 | 15.899 | 1.00 | 30.99 | A |
| ATOM | 316 | CE | LYS | A | 69 | 8.470 | 41.949 | 14.415 | 1.00 | 32.58 | A |
| ATOM | 317 | NZ | LYS | A | 69 | 7.069 | 41.696 | 13.953 | 1.00 | 33.89 | A |
| ATOM | 318 | C | LYS | A | 69 | 13.399 | 41.087 | 16.440 | 1.00 | 23.31 | A |
| ATOM | 319 | O | LYS | A | 69 | 13.965 | 40.779 | 15.394 | 1.00 | 22.08 | A |
| ATOM | 320 | N | TYR | A | 70 | 13.627 | 40.463 | 17.590 | 1.00 | 22.98 | A |
| ATOM | 321 | CA | TYR | A | 70 | 14.558 | 39.347 | 17.659 | 1.00 | 23.39 | A |
| ATOM | 322 | CB | TYR | A | 70 | 15.955 | 39.838 | 18.069 | 1.00 | 22.09 | A |
| ATOM | 323 | CG | TYR | A | 70 | 16.033 | 40.441 | 19.454 | 1.00 | 21.39 | A |
| ATOM | 324 | CD1 | TYR | A | 70 | 16.250 | 39.641 | 20.577 | 1.00 | 21.26 | A |
| ATOM | 325 | CE1 | TYR | A | 70 | 16.301 | 40.193 | 21.854 | 1.00 | 21.28 | A |
| ATOM | 326 | CD2 | TYR | A | 70 | 15.870 | 41.810 | 19.646 | 1.00 | 20.94 | A |
| ATOM | 327 | CE2 | TYR | A | 70 | 15.916 | 42.371 | 20.915 | 1.00 | 21.13 | A |
| ATOM | 328 | CZ | TYR | A | 70 | 16.131 | 41.560 | 22.014 | 1.00 | 21.33 | A |
| ATOM | 329 | OH | TYR | A | 70 | 16.160 | 42.120 | 23.270 | 1.00 | 21.29 | A |
| ATOM | 330 | C | TYR | A | 70 | 14.067 | 38.291 | 18.639 | 1.00 | 24.36 | A |
| ATOM | 331 | O | TYR | A | 70 | 13.299 | 38.587 | 19.556 | 1.00 | 24.37 | A |
| ATOM | 332 | N | ASP | A | 71 | 14.506 | 37.058 | 18.426 | 1.00 | 25.28 | A |
| ATOM | 333 | CA | ASP | A | 71 | 14.134 | 35.952 | 19.295 | 1.00 | 26.59 | A |
| ATOM | 334 | CB | ASP | A | 71 | 14.108 | 34.650 | 18.491 | 1.00 | 27.54 | A |
| ATOM | 335 | CG | ASP | A | 71 | 13.865 | 33.430 | 19.358 | 1.00 | 28.68 | A |
| ATOM | 336 | OD1 | ASP | A | 71 | 13.325 | 33.583 | 20.474 | 1.00 | 29.32 | A |
| ATOM | 337 | OD2 | ASP | A | 71 | 14.208 | 32.315 | 18.913 | 1.00 | 29.61 | A |
| ATOM | 338 | C | ASP | A | 71 | 15.166 | 35.878 | 20.414 | 1.00 | 27.11 | A |
| ATOM | 339 | O | ASP | A | 71 | 16.324 | 35.542 | 20.181 | 1.00 | 27.07 | A |
| ATOM | 340 | N | PRO | A | 72 | 14.758 | 36.205 | 21.650 | 1.00 | 27.95 | A |
| ATOM | 341 | CD | PRO | A | 72 | 13.382 | 36.467 | 22.102 | 1.00 | 28.09 | A |
| ATOM | 342 | CA | PRO | A | 72 | 15.682 | 36.167 | 22.788 | 1.00 | 28.21 | A |
| ATOM | 343 | CB | PRO | A | 72 | 14.777 | 36.487 | 23.981 | 1.00 | 28.36 | A |
| ATOM | 344 | CG | PRO | A | 72 | 13.431 | 36.001 | 23.536 | 1.00 | 29.13 | A |
| ATOM | 345 | C | PRO | A | 72 | 16.430 | 34.848 | 22.949 | 1.00 | 28.47 | A |
| ATOM | 346 | O | PRO | A | 72 | 17.544 | 34.819 | 23.478 | 1.00 | 28.39 | A |
| ATOM | 347 | N | LEU | A | 73 | 15.831 | 33.761 | 22.476 | 1.00 | 28.12 | A |
| ATOM | 348 | CA | LEU | A | 73 | 16.458 | 32.450 | 22.589 | 1.00 | 28.34 | A |
| ATOM | 349 | CB | LEU | A | 73 | 15.396 | 31.350 | 22.521 | 1.00 | 28.75 | A |
| ATOM | 350 | CG | LEU | A | 73 | 14.394 | 31.348 | 23.678 | 1.00 | 29.26 | A |
| ATOM | 351 | CD1 | LEU | A | 73 | 13.386 | 30.228 | 23.480 | 1.00 | 29.98 | A |
| ATOM | 352 | CD2 | LEU | A | 73 | 15.137 | 31.176 | 24.999 | 1.00 | 30.07 | A |
| ATOM | 353 | C | LEU | A | 73 | 17.526 | 32.200 | 21.530 | 1.00 | 28.03 | A |
| ATOM | 354 | O | LEU | A | 73 | 18.172 | 31.155 | 21.531 | 1.00 | 27.41 | A |
| ATOM | 355 | N | LYS | A | 74 | 17.717 | 33.159 | 20.629 | 1.00 | 27.84 | A |
| ATOM | 356 | CA | LYS | A | 74 | 18.719 | 33.010 | 19.580 | 1.00 | 28.11 | A |
| ATOM | 357 | CB | LYS | A | 74 | 18.719 | 34.238 | 18.669 | 1.00 | 28.49 | A |
| ATOM | 358 | CG | LYS | A | 74 | 19.670 | 34.132 | 17.489 | 1.00 | 29.82 | A |
| ATOM | 359 | CD | LYS | A | 74 | 19.495 | 35.303 | 16.537 | 1.00 | 30.64 | A |
| ATOM | 360 | CE | LYS | A | 74 | 20.364 | 35.143 | 15.302 | 1.00 | 31.81 | A |
| ATOM | 361 | NZ | LYS | A | 74 | 20.171 | 36.267 | 14.342 | 1.00 | 31.98 | A |
| ATOM | 362 | C | LYS | A | 74 | 20.107 | 32.821 | 20.188 | 1.00 | 28.09 | A |
| ATOM | 363 | O | LYS | A | 74 | 20.905 | 32.019 | 19.708 | 1.00 | 27.62 | A |
| ATOM | 364 | N | TYR | A | 75 | 20.390 | 33.567 | 21.249 | 1.00 | 28.79 | A |
| ATOM | 365 | CA | TYR | A | 75 | 21.678 | 33.470 | 21.917 | 1.00 | 29.44 | A |
| ATOM | 366 | CB | TYR | A | 75 | 22.267 | 34.870 | 22.135 | 1.00 | 30.25 | A |
| ATOM | 367 | CG | TYR | A | 75 | 22.560 | 35.593 | 20.839 | 1.00 | 31.19 | A |
| ATOM | 368 | CD1 | TYR | A | 75 | 21.682 | 36.552 | 20.330 | 1.00 | 32.32 | A |
| ATOM | 369 | CE1 | TYR | A | 75 | 21.919 | 37.167 | 19.097 | 1.00 | 32.54 | A |
| ATOM | 370 | CD2 | TYR | A | 75 | 23.685 | 35.268 | 20.085 | 1.00 | 31.91 | A |
| ATOM | 371 | CE2 | TYR | A | 75 | 23.929 | 35.871 | 18.854 | 1.00 | 33.09 | A |
| ATOM | 372 | CZ | TYR | A | 75 | 23.043 | 36.816 | 18.365 | 1.00 | 33.06 | A |
| ATOM | 373 | OH | TYR | A | 75 | 23.281 | 37.385 | 17.133 | 1.00 | 34.74 | A |
| ATOM | 374 | C | TYR | A | 75 | 21.533 | 32.738 | 23.246 | 1.00 | 29.32 | A |
| ATOM | 375 | O | TYR | A | 75 | 20.536 | 32.903 | 23.947 | 1.00 | 28.56 | A |
| ATOM | 376 | N | ASN | A | 76 | 22.524 | 31.914 | 23.573 | 1.00 | 29.65 | A |
| ATOM | 377 | CA | ASN | A | 76 | 22.515 | 31.153 | 24.817 | 1.00 | 30.92 | A |
| ATOM | 378 | CB | ASN | A | 76 | 21.679 | 29.879 | 24.663 | 1.00 | 31.88 | A |
| ATOM | 379 | CG | ASN | A | 76 | 22.350 | 28.844 | 23.789 | 1.00 | 32.89 | A |
| ATOM | 380 | OD1 | ASN | A | 76 | 22.597 | 29.077 | 22.610 | 1.00 | 34.45 | A |
| ATOM | 381 | ND2 | ASN | A | 76 | 22.652 | 27.686 | 24.368 | 1.00 | 35.12 | A |
| ATOM | 382 | C | ASN | A | 76 | 23.940 | 30.786 | 25.217 | 1.00 | 31.31 | A |
| ATOM | 383 | O | ASN | A | 76 | 24.898 | 31.163 | 24.544 | 1.00 | 31.17 | A |
| ATOM | 384 | N | ALA | A | 77 | 24.071 | 30.042 | 26.311 | 1.00 | 31.88 | A |
| ATOM | 385 | CA | ALA | A | 77 | 25.377 | 29.634 | 26.820 | 1.00 | 32.55 | A |
| ATOM | 386 | CB | ALA | A | 77 | 25.197 | 28.651 | 27.972 | 1.00 | 33.07 | A |
| ATOM | 387 | C | ALA | A | 77 | 26.297 | 29.028 | 25.763 | 1.00 | 33.01 | A |
| ATOM | 388 | O | ALA | A | 77 | 27.516 | 29.186 | 25.831 | 1.00 | 33.48 | A |
| ATOM | 389 | N | HIS | A | 78 | 25.718 | 28.340 | 24.785 | 1.00 | 33.14 | A |
| ATOM | 390 | CA | HIS | A | 78 | 26.512 | 27.700 | 23.741 | 1.00 | 33.71 | A |
| ATOM | 391 | CB | HIS | A | 78 | 25.869 | 26.367 | 23.349 | 1.00 | 35.33 | A |
| ATOM | 392 | CG | HIS | A | 78 | 25.613 | 25.459 | 24.511 | 1.00 | 37.19 | A |
| ATOM | 393 | CD2 | HIS | A | 78 | 24.467 | 24.910 | 24.978 | 1.00 | 37.95 | A |
| ATOM | 394 | ND1 | HIS | A | 78 | 26.616 | 25.029 | 25.354 | 1.00 | 38.44 | A |
| ATOM | 395 | CE1 | HIS | A | 78 | 26.098 | 24.255 | 26.291 | 1.00 | 39.01 | A |
| ATOM | 396 | NE2 | HIS | A | 78 | 24.796 | 24.166 | 26.085 | 1.00 | 38.98 | A |
| ATOM | 397 | C | HIS | A | 78 | 26.689 | 28.570 | 22.501 | 1.00 | 32.69 | A |
| ATOM | 398 | O | HIS | A | 78 | 27.445 | 28.221 | 21.594 | 1.00 | 32.79 | A |
| ATOM | 399 | N | HIS | A | 79 | 25.997 | 29.703 | 22.471 | 1.00 | 31.30 | A |
| ATOM | 400 | CA | HIS | A | 79 | 26.075 | 30.617 | 21.337 | 1.00 | 29.39 | A |
| ATOM | 401 | CB | HIS | A | 79 | 25.026 | 30.220 | 20.294 | 1.00 | 29.56 | A |
| ATOM | 402 | CG | HIS | A | 79 | 25.097 | 31.011 | 19.026 | 1.00 | 30.05 | A |
| ATOM | 403 | CD2 | HIS | A | 79 | 25.904 | 30.892 | 17.945 | 1.00 | 30.18 | A |
| ATOM | 404 | ND1 | HIS | A | 79 | 24.269 | 32.083 | 18.769 | 1.00 | 30.14 | A |
| ATOM | 405 | CE1 | HIS | A | 79 | 24.564 | 32.590 | 17.585 | 1.00 | 30.18 | A |
| ATOM | 406 | NE2 | HIS | A | 79 | 25.552 | 31.886 | 17.064 | 1.00 | 29.64 | A |
| ATOM | 407 | C | HIS | A | 79 | 25.848 | 32.049 | 21.821 | 1.00 | 27.72 | A |
| ATOM | 408 | O | HIS | A | 79 | 24.724 | 32.546 | 21.818 | 1.00 | 27.38 | A |
| ATOM | 409 | N | LYS | A | 80 | 26.929 | 32.701 | 22.239 | 1.00 | 25.74 | A |
| ATOM | 410 | CA | LYS | A | 80 | 26.864 | 34.067 | 22.751 | 1.00 | 24.30 | A |
| ATOM | 411 | CB | LYS | A | 80 | 27.894 | 34.265 | 23.864 | 1.00 | 25.55 | A |
| ATOM | 412 | CG | LYS | A | 80 | 27.771 | 33.315 | 25.047 | 1.00 | 27.24 | A |
| ATOM | 413 | CD | LYS | A | 80 | 28.848 | 33.636 | 26.074 | 1.00 | 28.76 | A |
| ATOM | 414 | CE | LYS | A | 80 | 28.801 | 32.686 | 27.260 | 1.00 | 30.53 | A |
| ATOM | 415 | NZ | LYS | A | 80 | 29.897 | 32.984 | 28.235 | 1.00 | 31.64 | A |
| ATOM | 416 | C | LYS | A | 80 | 27.117 | 35.130 | 21.687 | 1.00 | 23.05 | A |
| ATOM | 417 | O | LYS | A | 80 | 27.735 | 34.861 | 20.655 | 1.00 | 21.91 | A |
| ATOM | 418 | N | LEU | A | 81 | 26.634 | 36.340 | 21.961 | 1.00 | 20.81 | A |
| ATOM | 419 | CA | LEU | A | 81 | 26.826 | 37.476 | 21.069 | 1.00 | 19.46 | A |
| ATOM | 420 | CB | LEU | A | 81 | 25.639 | 38.444 | 21.163 | 1.00 | 18.84 | A |
| ATOM | 421 | CG | LEU | A | 81 | 25.719 | 39.706 | 20.294 | 1.00 | 18.97 | A |
| ATOM | 422 | CD1 | LEU | A | 81 | 25.692 | 39.316 | 18.819 | 1.00 | 18.21 | A |
| ATOM | 423 | CD2 | LEU | A | 81 | 24.558 | 40.637 | 20.616 | 1.00 | 18.43 | A |
| ATOM | 424 | C | LEU | A | 81 | 28.097 | 38.179 | 21.532 | 1.00 | 19.09 | A |
| ATOM | 425 | O | LEU | A | 81 | 28.168 | 38.669 | 22.660 | 1.00 | 18.47 | A |
| ATOM | 426 | N | LYS | A | 82 | 29.108 | 38.198 | 20.670 | 1.00 | 18.24 | A |
| ATOM | 427 | CA | LYS | A | 82 | 30.379 | 38.844 | 20.984 | 1.00 | 18.10 | A |
| ATOM | 428 | CB | LYS | A | 82 | 31.523 | 38.130 | 20.258 | 1.00 | 20.34 | A |
| ATOM | 429 | CG | LYS | A | 82 | 31.736 | 36.693 | 20.723 | 1.00 | 23.59 | A |
| ATOM | 430 | CD | LYS | A | 82 | 32.626 | 35.899 | 19.769 | 1.00 | 26.71 | A |
| ATOM | 431 | CE | LYS | A | 82 | 34.047 | 36.436 | 19.723 | 1.00 | 28.41 | A |
| ATOM | 432 | NZ | LYS | A | 82 | 34.880 | 35.658 | 18.761 | 1.00 | 31.12 | A |
| ATOM | 433 | C | LYS | A | 82 | 30.283 | 40.289 | 20.517 | 1.00 | 17.55 | A |
| ATOM | 434 | O | LYS | A | 82 | 30.112 | 40.550 | 19.327 | 1.00 | 16.98 | A |
| ATOM | 435 | N | VAL | A | 83 | 30.392 | 41.226 | 21.454 | 1.00 | 15.95 | A |
| ATOM | 436 | CA | VAL | A | 83 | 30.285 | 42.638 | 21.119 | 1.00 | 15.21 | A |
| ATOM | 437 | CB | VAL | A | 83 | 29.253 | 43.349 | 22.035 | 1.00 | 14.49 | A |
| ATOM | 438 | CG1 | VAL | A | 83 | 29.126 | 44.814 | 21.648 | 1.00 | 13.69 | A |
| ATOM | 439 | CG2 | VAL | A | 83 | 27.895 | 42.658 | 21.926 | 1.00 | 15.01 | A |
| ATOM | 440 | C | VAL | A | 83 | 31.615 | 43.375 | 21.229 | 1.00 | 15.13 | A |
| ATOM | 441 | O | VAL | A | 83 | 32.297 | 43.302 | 22.252 | 1.00 | 15.11 | A |
| ATOM | 442 | N | PHE | A | 84 | 31.976 | 44.084 | 20.163 | 1.00 | 15.08 | A |
| ATOM | 443 | CA | PHE | A | 84 | 33.207 | 44.868 | 20.142 | 1.00 | 14.60 | A |
| ATOM | 444 | CB | PHE | A | 84 | 34.081 | 44.489 | 18.944 | 1.00 | 15.76 | A |
| ATOM | 445 | CG | PHE | A | 84 | 34.765 | 43.163 | 19.085 | 1.00 | 17.05 | A |
| ATOM | 446 | CD1 | PHE | A | 84 | 34.321 | 42.055 | 18.371 | 1.00 | 17.71 | A |
| ATOM | 447 | CD2 | PHE | A | 84 | 35.859 | 43.021 | 19.935 | 1.00 | 18.34 | A |
| ATOM | 448 | CE1 | PHE | A | 84 | 34.961 | 40.819 | 18.500 | 1.00 | 18.55 | A |
| ATOM | 449 | CE2 | PHE | A | 84 | 36.507 | 41.795 | 20.073 | 1.00 | 18.71 | A |
| ATOM | 450 | CZ | PHE | A | 84 | 36.058 | 40.690 | 19.354 | 1.00 | 19.03 | A |
| ATOM | 451 | C | PHE | A | 84 | 32.876 | 46.355 | 20.061 | 1.00 | 14.25 | A |
| ATOM | 452 | O | PHE | A | 84 | 32.342 | 46.819 | 19.054 | 1.00 | 14.04 | A |
| ATOM | 453 | N | VAL | A | 85 | 33.183 | 47.085 | 21.130 | 1.00 | 13.03 | A |
| ATOM | 454 | CA | VAL | A | 85 | 32.955 | 48.527 | 21.193 | 1.00 | 13.03 | A |
| ATOM | 455 | CB | VAL | A | 85 | 32.596 | 48.976 | 22.629 | 1.00 | 12.58 | A |
| ATOM | 456 | CG1 | VAL | A | 85 | 32.408 | 50.493 | 22.680 | 1.00 | 13.25 | A |
| ATOM | 457 | CG2 | VAL | A | 85 | 31.318 | 48.272 | 23.072 | 1.00 | 13.56 | A |
| ATOM | 458 | C | VAL | A | 85 | 34.267 | 49.165 | 20.762 | 1.00 | 11.93 | A |
| ATOM | 459 | O | VAL | A | 85 | 35.280 | 49.049 | 21.451 | 1.00 | 12.19 | A |
| ATOM | 460 | N | VAL | A | 86 | 34.240 | 49.840 | 19.618 | 1.00 | 11.92 | A |
| ATOM | 461 | CA | VAL | A | 86 | 35.442 | 50.442 | 19.058 | 1.00 | 11.60 | A |
| ATOM | 462 | CB | VAL | A | 86 | 35.580 | 50.032 | 17.571 | 1.00 | 12.47 | A |
| ATOM | 463 | CG1 | VAL | A | 86 | 36.907 | 50.523 | 17.002 | 1.00 | 11.40 | A |
| ATOM | 464 | CG2 | VAL | A | 86 | 35.458 | 48.511 | 17.442 | 1.00 | 13.05 | A |
| ATOM | 465 | C | VAL | A | 86 | 35.508 | 51.967 | 19.168 | 1.00 | 11.18 | A |
| ATOM | 466 | O | VAL | A | 86 | 34.875 | 52.683 | 18.393 | 1.00 | 10.78 | A |
| ATOM | 467 | N | PRO | A | 87 | 36.292 | 52.478 | 20.133 | 1.00 | 10.83 | A |
| ATOM | 468 | CD | PRO | A | 87 | 37.015 | 51.722 | 21.169 | 1.00 | 10.63 | A |
| ATOM | 469 | CA | PRO | A | 87 | 36.448 | 53.923 | 20.344 | 1.00 | 10.81 | A |
| ATOM | 470 | CB | PRO | A | 87 | 37.307 | 54.000 | 21.610 | 1.00 | 10.48 | A |
| ATOM | 471 | CG | PRO | A | 87 | 37.008 | 52.695 | 22.311 | 1.00 | 10.82 | A |
| ATOM | 472 | C | PRO | A | 87 | 37.145 | 54.569 | 19.146 | 1.00 | 10.84 | A |
| ATOM | 473 | O | PRO | A | 87 | 38.123 | 54.023 | 18.624 | 1.00 | 10.85 | A |
| ATOM | 474 | N | HIS | A | 88 | 36.646 | 55.725 | 18.714 | 1.00 | 10.80 | A |
| ATOM | 475 | CA | HIS | A | 88 | 37.235 | 56.427 | 17.577 | 1.00 | 11.53 | A |
| ATOM | 476 | CB | HIS | A | 88 | 36.662 | 55.885 | 16.257 | 1.00 | 10.95 | A |
| ATOM | 477 | CG | HIS | A | 88 | 35.211 | 56.191 | 16.051 | 1.00 | 11.87 | A |
| ATOM | 478 | CD2 | HIS | A | 88 | 34.098 | 55.515 | 16.420 | 1.00 | 11.54 | A |
| ATOM | 479 | ND1 | HIS | A | 88 | 34.773 | 57.323 | 15.397 | 1.00 | 10.84 | A |
| ATOM | 480 | CE1 | HIS | A | 88 | 33.453 | 57.330 | 15.371 | 1.00 | 11.92 | A |
| ATOM | 481 | NE2 | HIS | A | 88 | 33.018 | 56.244 | 15.986 | 1.00 | 11.27 | A |
| ATOM | 482 | C | HIS | A | 88 | 37.015 | 57.933 | 17.666 | 1.00 | 12.18 | A |
| ATOM | 483 | O | HIS | A | 88 | 36.203 | 58.417 | 18.459 | 1.00 | 11.77 | A |
| ATOM | 484 | N | SER | A | 89 | 37.753 | 58.670 | 16.845 | 1.00 | 11.89 | A |
| ATOM | 485 | CA | SER | A | 89 | 37.671 | 60.122 | 16.833 | 1.00 | 11.62 | A |
| ATOM | 486 | CB | SER | A | 89 | 38.775 | 60.702 | 17.728 | 1.00 | 10.98 | A |
| ATOM | 487 | OG | SER | A | 89 | 38.737 | 62.117 | 17.764 | 1.00 | 11.39 | A |
| ATOM | 488 | C | SER | A | 89 | 37.852 | 60.577 | 15.393 | 1.00 | 11.91 | A |
| ATOM | 489 | O | SER | A | 89 | 38.928 | 60.417 | 14.815 | 1.00 | 12.41 | A |
| ATOM | 490 | N | HIS | A | 90 | 36.792 | 61.130 | 14.814 | 1.00 | 11.46 | A |
| ATOM | 491 | CA | HIS | A | 90 | 36.835 | 61.592 | 13.432 | 1.00 | 11.67 | A |
| ATOM | 492 | CB | HIS | A | 90 | 35.415 | 61.640 | 12.859 | 1.00 | 10.52 | A |
| ATOM | 493 | CG | HIS | A | 90 | 35.368 | 61.922 | 11.391 | 1.00 | 10.05 | A |
| ATOM | 494 | CD2 | HIS | A | 90 | 34.794 | 62.930 | 10.695 | 1.00 | 9.72 | A |
| ATOM | 495 | ND1 | HIS | A | 90 | 35.986 | 61.116 | 10.460 | 1.00 | 10.72 | A |
| ATOM | 496 | CE1 | HIS | A | 90 | 35.795 | 61.615 | 9.253 | 1.00 | 10.25 | A |
| ATOM | 497 | NE2 | HIS | A | 90 | 35.074 | 62.716 | 9.368 | 1.00 | 9.97 | A |
| ATOM | 498 | C | HIS | A | 90 | 37.491 | 62.969 | 13.355 | 1.00 | 11.84 | A |
| ATOM | 499 | O | HIS | A | 90 | 36.912 | 63.971 | 13.787 | 1.00 | 11.75 | A |
| ATOM | 500 | N | ASN | A | 91 | 38.704 | 63.005 | 12.809 | 1.00 | 11.84 | A |
| ATOM | 501 | CA | ASN | A | 91 | 39.466 | 64.243 | 12.680 | 1.00 | 12.03 | A |
| ATOM | 502 | CB | ASN | A | 91 | 40.857 | 64.082 | 13.304 | 1.00 | 12.04 | A |
| ATOM | 503 | CG | ASN | A | 91 | 40.812 | 63.945 | 14.810 | 1.00 | 12.39 | A |
| ATOM | 504 | OD1 | ASN | A | 91 | 40.213 | 63.011 | 15.348 | 1.00 | 14.18 | A |
| ATOM | 505 | ND2 | ASN | A | 91 | 41.451 | 64.877 | 15.503 | 1.00 | 10.68 | A |
| ATOM | 506 | C | ASN | A | 91 | 39.628 | 64.671 | 11.226 | 1.00 | 12.39 | A |
| ATOM | 507 | O | ASN | A | 91 | 40.322 | 64.014 | 10.450 | 1.00 | 12.84 | A |
| ATOM | 508 | N | ASP | A | 92 | 38.996 | 65.783 | 10.873 | 1.00 | 11.57 | A |
| ATOM | 509 | CA | ASP | A | 92 | 39.063 | 66.308 | 9.517 | 1.00 | 12.43 | A |
| ATOM | 510 | CB | ASP | A | 92 | 37.827 | 67.155 | 9.224 | 1.00 | 11.83 | A |
| ATOM | 511 | CG | ASP | A | 92 | 36.555 | 66.377 | 9.378 | 1.00 | 12.30 | A |
| ATOM | 512 | OD1 | ASP | A | 92 | 36.335 | 65.462 | 8.565 | 1.00 | 12.29 | A |
| ATOM | 513 | OD2 | ASP | A | 92 | 35.791 | 66.671 | 10.319 | 1.00 | 13.77 | A |
| ATOM | 514 | C | ASP | A | 92 | 40.293 | 67.168 | 9.282 | 1.00 | 12.14 | A |
| ATOM | 515 | O | ASP | A | 92 | 40.522 | 68.136 | 10.005 | 1.00 | 12.62 | A |
| ATOM | 516 | N | PRO | A | 93 | 41.115 | 66.810 | 8.284 | 1.00 | 12.65 | A |
| ATOM | 517 | CD | PRO | A | 93 | 41.195 | 65.488 | 7.637 | 1.00 | 12.43 | A |
| ATOM | 518 | CA | PRO | A | 93 | 42.311 | 67.603 | 7.988 | 1.00 | 12.86 | A |
| ATOM | 519 | CB | PRO | A | 93 | 43.108 | 66.697 | 7.052 | 1.00 | 12.57 | A |
| ATOM | 520 | CG | PRO | A | 93 | 42.686 | 65.311 | 7.478 | 1.00 | 13.74 | A |
| ATOM | 521 | C | PRO | A | 93 | 41.823 | 68.876 | 7.290 | 1.00 | 13.17 | A |
| ATOM | 522 | O | PRO | A | 93 | 42.157 | 69.139 | 6.132 | 1.00 | 13.70 | A |
| ATOM | 523 | N | GLY | A | 94 | 41.008 | 69.641 | 8.010 | 1.00 | 12.62 | A |
| ATOM | 524 | CA | GLY | A | 94 | 40.446 | 70.868 | 7.481 | 1.00 | 11.94 | A |
| ATOM | 525 | C | GLY | A | 94 | 38.965 | 70.725 | 7.160 | 1.00 | 12.22 | A |
| ATOM | 526 | O | GLY | A | 94 | 38.530 | 69.697 | 6.636 | 1.00 | 10.48 | A |
| ATOM | 527 | N | TRP | A | 95 | 38.191 | 71.751 | 7.506 | 1.00 | 12.07 | A |
| ATOM | 528 | CA | TRP | A | 95 | 36.755 | 71.808 | 7.232 | 1.00 | 13.08 | A |
| ATOM | 529 | CB | TRP | A | 95 | 35.964 | 70.737 | 7.996 | 1.00 | 12.61 | A |
| ATOM | 530 | CG | TRP | A | 95 | 34.480 | 70.807 | 7.674 | 1.00 | 12.37 | A |
| ATOM | 531 | CD2 | TRP | A | 95 | 33.381 | 70.521 | 8.556 | 1.00 | 12.36 | A |
| ATOM | 532 | CE2 | TRP | A | 95 | 32.191 | 70.701 | 7.812 | 1.00 | 11.92 | A |
| ATOM | 533 | CE3 | TRP | A | 95 | 33.285 | 70.128 | 9.900 | 1.00 | 12.35 | A |
| ATOM | 534 | CD1 | TRP | A | 95 | 33.923 | 71.138 | 6.469 | 1.00 | 12.10 | A |
| ATOM | 535 | NE1 | TRP | A | 95 | 32.551 | 71.079 | 6.545 | 1.00 | 12.46 | A |
| ATOM | 536 | CZ2 | TRP | A | 95 | 30.919 | 70.501 | 8.366 | 1.00 | 12.42 | A |
| ATOM | 537 | CZ3 | TRP | A | 95 | 32.015 | 69.928 | 10.453 | 1.00 | 12.35 | A |
| ATOM | 538 | CH2 | TRP | A | 95 | 30.853 | 70.116 | 9.684 | 1.00 | 12.53 | A |
| ATOM | 539 | C | TRP | A | 95 | 36.241 | 73.191 | 7.623 | 1.00 | 13.99 | A |
| ATOM | 540 | O | TRP | A | 95 | 36.115 | 74.066 | 6.770 | 1.00 | 13.09 | A |
| ATOM | 541 | N | ILE | A | 96 | 35.938 | 73.385 | 8.906 | 1.00 | 15.45 | A |
| ATOM | 542 | CA | ILE | A | 96 | 35.475 | 74.693 | 9.365 | 1.00 | 16.71 | A |
| ATOM | 543 | CB | ILE | A | 96 | 34.333 | 74.581 | 10.399 | 1.00 | 18.64 | A |
| ATOM | 544 | CG2 | ILE | A | 96 | 33.083 | 74.028 | 9.719 | 1.00 | 18.36 | A |
| ATOM | 545 | CG1 | ILE | A | 96 | 34.760 | 73.709 | 11.576 | 1.00 | 20.42 | A |
| ATOM | 546 | CD1 | ILE | A | 96 | 33.740 | 73.664 | 12.692 | 1.00 | 23.56 | A |
| ATOM | 547 | C | ILE | A | 96 | 36.658 | 75.456 | 9.955 | 1.00 | 16.73 | A |
| ATOM | 548 | O | ILE | A | 96 | 36.567 | 76.644 | 10.261 | 1.00 | 16.70 | A |
| ATOM | 549 | N | GLN | A | 97 | 37.768 | 74.741 | 10.107 | 1.00 | 16.15 | A |
| ATOM | 550 | CA | GLN | A | 97 | 39.028 | 75.295 | 10.585 | 1.00 | 16.16 | A |
| ATOM | 551 | CB | GLN | A | 97 | 39.325 | 74.865 | 12.027 | 1.00 | 18.11 | A |
| ATOM | 552 | CG | GLN | A | 97 | 38.431 | 75.503 | 13.087 | 1.00 | 22.05 | A |
| ATOM | 553 | CD | GLN | A | 97 | 38.907 | 75.206 | 14.504 | 1.00 | 25.07 | A |
| ATOM | 554 | OE1 | GLN | A | 97 | 40.041 | 75.525 | 14.872 | 1.00 | 27.71 | A |
| ATOM | 555 | NE2 | GLN | A | 97 | 38.041 | 74.592 | 15.306 | 1.00 | 27.57 | A |
| ATOM | 556 | C | GLN | A | 97 | 40.069 | 74.685 | 9.649 | 1.00 | 14.57 | A |
| ATOM | 557 | O | GLN | A | 97 | 39.795 | 73.683 | 8.988 | 1.00 | 13.77 | A |
| ATOM | 558 | N | THR | A | 98 | 41.249 | 75.283 | 9.574 | 1.00 | 13.40 | A |
| ATOM | 559 | CA | THR | A | 98 | 42.293 | 74.742 | 8.713 | 1.00 | 12.50 | A |
| ATOM | 560 | CB | THR | A | 98 | 43.402 | 75.763 | 8.456 | 1.00 | 12.05 | A |
| ATOM | 561 | OG1 | THR | A | 98 | 44.038 | 76.073 | 9.700 | 1.00 | 13.52 | A |
| ATOM | 562 | CG2 | THR | A | 98 | 42.841 | 77.040 | 7.836 | 1.00 | 11.40 | A |
| ATOM | 563 | C | THR | A | 98 | 42.942 | 73.551 | 9.411 | 1.00 | 12.44 | A |
| ATOM | 564 | O | THR | A | 98 | 42.713 | 73.312 | 10.601 | 1.00 | 12.49 | A |
| ATOM | 565 | N | PHE | A | 99 | 43.754 | 72.810 | 8.666 | 1.00 | 11.73 | A |
| ATOM | 566 | CA | PHE | A | 99 | 44.462 | 71.665 | 9.221 | 1.00 | 12.23 | A |
| ATOM | 567 | CB | PHE | A | 99 | 45.472 | 71.120 | 8.204 | 1.00 | 11.49 | A |
| ATOM | 568 | CG | PHE | A | 99 | 46.350 | 70.017 | 8.745 | 1.00 | 12.06 | A |
| ATOM | 569 | CD1 | PHE | A | 99 | 45.941 | 68.687 | 8.686 | 1.00 | 11.94 | A |
| ATOM | 570 | CD2 | PHE | A | 99 | 47.589 | 70.312 | 9.314 | 1.00 | 11.54 | A |
| ATOM | 571 | CE1 | PHE | A | 99 | 46.752 | 67.663 | 9.182 | 1.00 | 12.73 | A |
| ATOM | 572 | CE2 | PHE | A | 99 | 48.408 | 69.298 | 9.814 | 1.00 | 12.59 | A |
| ATOM | 573 | CZ | PHE | A | 99 | 47.988 | 67.968 | 9.747 | 1.00 | 12.47 | A |
| ATOM | 574 | C | PHE | A | 99 | 45.211 | 72.088 | 10.486 | 1.00 | 12.42 | A |
| ATOM | 575 | O | PHE | A | 99 | 45.055 | 71.479 | 11.540 | 1.00 | 12.95 | A |
| ATOM | 576 | N | GLU | A | 100 | 46.026 | 73.134 | 10.370 | 1.00 | 13.15 | A |
| ATOM | 577 | CA | GLU | A | 100 | 46.818 | 73.617 | 11.502 | 1.00 | 14.12 | A |
| ATOM | 578 | CB | GLU | A | 100 | 47.789 | 74.708 | 11.034 | 1.00 | 14.54 | A |
| ATOM | 579 | CG | GLU | A | 100 | 48.842 | 75.113 | 12.062 | 1.00 | 15.92 | A |
| ATOM | 580 | CD | GLU | A | 100 | 49.753 | 73.965 | 12.474 | 1.00 | 17.24 | A |
| ATOM | 581 | OE1 | GLU | A | 100 | 49.923 | 73.012 | 11.680 | 1.00 | 16.33 | A |
| ATOM | 582 | OE2 | GLU | A | 100 | 50.316 | 74.028 | 13.591 | 1.00 | 18.05 | A |
| ATOM | 583 | C | GLU | A | 100 | 45.973 | 74.135 | 12.666 | 1.00 | 14.27 | A |
| ATOM | 584 | O | GLU | A | 100 | 46.330 | 73.936 | 13.827 | 1.00 | 14.15 | A |
| ATOM | 585 | N | GLU | A | 101 | 44.860 | 74.798 | 12.364 | 1.00 | 14.44 | A |
| ATOM | 586 | CA | GLU | A | 101 | 43.989 | 75.313 | 13.420 | 1.00 | 14.32 | A |
| ATOM | 587 | CB | GLU | A | 101 | 42.850 | 76.150 | 12.823 | 1.00 | 15.58 | A |
| ATOM | 588 | CG | GLU | A | 101 | 43.314 | 77.445 | 12.156 | 1.00 | 16.71 | A |
| ATOM | 589 | CD | GLU | A | 101 | 42.163 | 78.275 | 11.601 | 1.00 | 17.74 | A |
| ATOM | 590 | OE1 | GLU | A | 101 | 41.227 | 77.690 | 11.020 | 1.00 | 16.12 | A |
| ATOM | 591 | OE2 | GLU | A | 101 | 42.205 | 79.518 | 11.736 | 1.00 | 18.27 | A |
| ATOM | 592 | C | GLU | A | 101 | 43.416 | 74.144 | 14.224 | 1.00 | 14.11 | A |
| ATOM | 593 | O | GLU | A | 101 | 43.411 | 74.169 | 15.456 | 1.00 | 13.45 | A |
| ATOM | 594 | N | TYR | A | 102 | 42.930 | 73.120 | 13.526 | 1.00 | 13.09 | A |
| ATOM | 595 | CA | TYR | A | 102 | 42.385 | 71.947 | 14.205 | 1.00 | 13.59 | A |
| ATOM | 596 | CB | TYR | A | 102 | 41.819 | 70.934 | 13.210 | 1.00 | 13.65 | A |
| ATOM | 597 | CG | TYR | A | 102 | 40.407 | 71.173 | 12.737 | 1.00 | 13.71 | A |
| ATOM | 598 | CD1 | TYR | A | 102 | 39.354 | 71.343 | 13.641 | 1.00 | 14.39 | A |
| ATOM | 599 | CE1 | TYR | A | 102 | 38.030 | 71.454 | 13.190 | 1.00 | 14.14 | A |
| ATOM | 600 | CD2 | TYR | A | 102 | 40.106 | 71.131 | 11.376 | 1.00 | 13.81 | A |
| ATOM | 601 | CE2 | TYR | A | 102 | 38.806 | 71.240 | 10.921 | 1.00 | 13.84 | A |
| ATOM | 602 | CZ | TYR | A | 102 | 37.771 | 71.399 | 11.826 | 1.00 | 13.69 | A |
| ATOM | 603 | OH | TYR | A | 102 | 36.487 | 71.490 | 11.343 | 1.00 | 13.18 | A |
| ATOM | 604 | C | TYR | A | 102 | 43.482 | 71.247 | 14.999 | 1.00 | 13.51 | A |
| ATOM | 605 | O | TYR | A | 102 | 43.258 | 70.795 | 16.123 | 1.00 | 12.77 | A |
| ATOM | 606 | N | TYR | A | 103 | 44.663 | 71.136 | 14.402 | 1.00 | 13.52 | A |
| ATOM | 607 | CA | TYR | A | 103 | 45.763 | 70.471 | 15.081 | 1.00 | 14.48 | A |
| ATOM | 608 | CB | TYR | A | 103 | 47.024 | 70.464 | 14.217 | 1.00 | 13.22 | A |
| ATOM | 609 | CG | TYR | A | 103 | 48.189 | 69.805 | 14.918 | 1.00 | 13.75 | A |
| ATOM | 610 | CD1 | TYR | A | 103 | 48.191 | 68.431 | 15.163 | 1.00 | 13.55 | A |
| ATOM | 611 | CE1 | TYR | A | 103 | 49.230 | 67.827 | 15.867 | 1.00 | 13.28 | A |
| ATOM | 612 | CD2 | TYR | A | 103 | 49.262 | 70.561 | 15.394 | 1.00 | 14.16 | A |
| ATOM | 613 | CE2 | TYR | A | 103 | 50.304 | 69.967 | 16.101 | 1.00 | 14.10 | A |
| ATOM | 614 | CZ | TYR | A | 103 | 50.281 | 68.601 | 16.335 | 1.00 | 14.07 | A |
| ATOM | 615 | OH | TYR | A | 103 | 51.301 | 68.009 | 17.045 | 1.00 | 14.60 | A |
| ATOM | 616 | C | TYR | A | 103 | 46.083 | 71.148 | 16.407 | 1.00 | 15.20 | A |
| ATOM | 617 | O | TYR | A | 103 | 46.206 | 70.489 | 17.438 | 1.00 | 15.39 | A |
| ATOM | 618 | N | GLN | A | 104 | 46.214 | 72.468 | 16.372 | 1.00 | 15.88 | A |
| ATOM | 619 | CA | GLN | A | 104 | 46.546 | 73.233 | 17.568 | 1.00 | 16.71 | A |
| ATOM | 620 | CB | GLN | A | 104 | 46.905 | 74.676 | 17.189 | 1.00 | 17.21 | A |
| ATOM | 621 | CG | GLN | A | 104 | 48.221 | 74.831 | 16.436 | 1.00 | 16.37 | A |
| ATOM | 622 | CD | GLN | A | 104 | 49.408 | 74.295 | 17.221 | 1.00 | 17.63 | A |
| ATOM | 623 | OE1 | GLN | A | 104 | 49.456 | 74.404 | 18.449 | 1.00 | 17.43 | A |
| ATOM | 624 | NE2 | GLN | A | 104 | 50.378 | 73.722 | 16.514 | 1.00 | 16.09 | A |
| ATOM | 625 | C | GLN | A | 104 | 45.438 | 73.259 | 18.610 | 1.00 | 17.34 | A |
| ATOM | 626 | O | GLN | A | 104 | 45.702 | 73.153 | 19.805 | 1.00 | 17.27 | A |
| ATOM | 627 | N | HIS | A | 105 | 44.197 | 73.392 | 18.157 | 1.00 | 17.76 | A |
| ATOM | 628 | CA | HIS | A | 105 | 43.063 | 73.479 | 19.068 | 1.00 | 18.88 | A |
| ATOM | 629 | CB | HIS | A | 105 | 41.928 | 74.271 | 18.408 | 1.00 | 21.57 | A |
| ATOM | 630 | CG | HIS | A | 105 | 42.350 | 75.595 | 17.851 | 1.00 | 24.72 | A |
| ATOM | 631 | CD2 | HIS | A | 105 | 43.543 | 76.237 | 17.874 | 1.00 | 26.28 | A |
| ATOM | 632 | ND1 | HIS | A | 105 | 41.486 | 76.419 | 17.161 | 1.00 | 26.75 | A |
| ATOM | 633 | CE1 | HIS | A | 105 | 42.129 | 77.510 | 16.783 | 1.00 | 27.01 | A |
| ATOM | 634 | NE2 | HIS | A | 105 | 43.378 | 77.424 | 17.203 | 1.00 | 27.01 | A |
| ATOM | 635 | C | HIS | A | 105 | 42.492 | 72.154 | 19.563 | 1.00 | 18.41 | A |
| ATOM | 636 | O | HIS | A | 105 | 42.010 | 72.076 | 20.692 | 1.00 | 18.04 | A |
| ATOM | 637 | N | ASP | A | 106 | 42.552 | 71.114 | 18.733 | 1.00 | 16.87 | A |
| ATOM | 638 | CA | ASP | A | 106 | 41.956 | 69.835 | 19.108 | 1.00 | 16.42 | A |
| ATOM | 639 | CB | ASP | A | 106 | 40.668 | 69.634 | 18.301 | 1.00 | 16.96 | A |
| ATOM | 640 | CG | ASP | A | 106 | 39.650 | 70.730 | 18.541 | 1.00 | 18.76 | A |
| ATOM | 641 | OD1 | ASP | A | 106 | 38.962 | 70.684 | 19.579 | 1.00 | 19.47 | A |
| ATOM | 642 | OD2 | ASP | A | 106 | 39.547 | 71.644 | 17.694 | 1.00 | 19.48 | A |
| ATOM | 643 | C | ASP | A | 106 | 42.792 | 68.567 | 18.978 | 1.00 | 15.43 | A |
| ATOM | 644 | O | ASP | A | 106 | 43.061 | 67.885 | 19.965 | 1.00 | 15.33 | A |
| ATOM | 645 | N | THR | A | 107 | 43.192 | 68.251 | 17.752 | 1.00 | 14.43 | A |
| ATOM | 646 | CA | THR | A | 107 | 43.931 | 67.027 | 17.470 | 1.00 | 13.03 | A |
| ATOM | 647 | CB | THR | A | 107 | 44.290 | 66.949 | 15.982 | 1.00 | 13.11 | A |
| ATOM | 648 | OG1 | THR | A | 107 | 43.104 | 67.149 | 15.203 | 1.00 | 11.86 | A |
| ATOM | 649 | CG2 | THR | A | 107 | 44.876 | 65.574 | 15.648 | 1.00 | 12.11 | A |
| ATOM | 650 | C | THR | A | 107 | 45.182 | 66.709 | 18.287 | 1.00 | 13.20 | A |
| ATOM | 651 | O | THR | A | 107 | 45.365 | 65.565 | 18.704 | 1.00 | 12.11 | A |
| ATOM | 652 | N | LYS | A | 108 | 46.053 | 67.685 | 18.515 | 1.00 | 12.87 | A |
| ATOM | 653 | CA | LYS | A | 108 | 47.254 | 67.364 | 19.280 | 1.00 | 13.87 | A |
| ATOM | 654 | CB | LYS | A | 108 | 48.252 | 68.534 | 19.277 | 1.00 | 14.10 | A |
| ATOM | 655 | CG | LYS | A | 108 | 47.860 | 69.751 | 20.090 | 1.00 | 15.29 | A |
| ATOM | 656 | CD | LYS | A | 108 | 48.944 | 70.823 | 19.971 | 1.00 | 15.77 | A |
| ATOM | 657 | CE | LYS | A | 108 | 48.719 | 71.975 | 20.937 | 1.00 | 16.23 | A |
| ATOM | 658 | NZ | LYS | A | 108 | 49.829 | 72.973 | 20.871 | 1.00 | 16.57 | A |
| ATOM | 659 | C | LYS | A | 108 | 46.891 | 66.964 | 20.706 | 1.00 | 13.55 | A |
| ATOM | 660 | O | LYS | A | 108 | 47.588 | 66.169 | 21.330 | 1.00 | 13.59 | A |
| ATOM | 661 | N | HIS | A | 109 | 45.790 | 67.505 | 21.213 | 1.00 | 14.16 | A |
| ATOM | 662 | CA | HIS | A | 109 | 45.343 | 67.179 | 22.565 | 1.00 | 14.77 | A |
| ATOM | 663 | CB | HIS | A | 109 | 44.353 | 68.232 | 23.050 | 1.00 | 16.40 | A |
| ATOM | 664 | CG | HIS | A | 109 | 44.924 | 69.614 | 23.077 | 1.00 | 18.18 | A |
| ATOM | 665 | CD2 | HIS | A | 109 | 44.655 | 70.706 | 22.324 | 1.00 | 18.81 | A |
| ATOM | 666 | ND1 | HIS | A | 109 | 45.937 | 69.981 | 23.939 | 1.00 | 19.58 | A |
| ATOM | 667 | CE1 | HIS | A | 109 | 46.266 | 71.241 | 23.714 | 1.00 | 19.97 | A |
| ATOM | 668 | NE2 | HIS | A | 109 | 45.503 | 71.703 | 22.739 | 1.00 | 20.15 | A |
| ATOM | 669 | C | HIS | A | 109 | 44.699 | 65.797 | 22.571 | 1.00 | 14.18 | A |
| ATOM | 670 | O | HIS | A | 109 | 44.864 | 65.024 | 23.515 | 1.00 | 13.72 | A |
| ATOM | 671 | N | ILE | A | 110 | 43.967 | 65.492 | 21.506 | 1.00 | 13.63 | A |
| ATOM | 672 | CA | ILE | A | 110 | 43.319 | 64.195 | 21.376 | 1.00 | 12.97 | A |
| ATOM | 673 | CB | ILE | A | 110 | 42.494 | 64.121 | 20.072 | 1.00 | 13.70 | A |
| ATOM | 674 | CG2 | ILE | A | 110 | 42.039 | 62.686 | 19.822 | 1.00 | 13.17 | A |
| ATOM | 675 | CG1 | ILE | A | 110 | 41.309 | 65.091 | 20.158 | 1.00 | 13.46 | A |
| ATOM | 676 | CD1 | ILE | A | 110 | 40.505 | 65.217 | 18.870 | 1.00 | 13.46 | A |
| ATOM | 677 | C | ILE | A | 110 | 44.378 | 63.096 | 21.355 | 1.00 | 13.01 | A |
| ATOM | 678 | O | ILE | A | 110 | 44.259 | 62.088 | 22.053 | 1.00 | 12.30 | A |
| ATOM | 679 | N | LEU | A | 111 | 45.419 | 63.295 | 20.557 | 1.00 | 12.48 | A |
| ATOM | 680 | CA | LEU | A | 111 | 46.479 | 62.303 | 20.455 | 1.00 | 13.22 | A |
| ATOM | 681 | CB | LEU | A | 111 | 47.348 | 62.584 | 19.222 | 1.00 | 13.48 | A |
| ATOM | 682 | CG | LEU | A | 111 | 46.624 | 62.258 | 17.910 | 1.00 | 13.21 | A |
| ATOM | 683 | CD1 | LEU | A | 111 | 47.448 | 62.722 | 16.714 | 1.00 | 12.52 | A |
| ATOM | 684 | CD2 | LEU | A | 111 | 46.359 | 60.755 | 17.848 | 1.00 | 13.63 | A |
| ATOM | 685 | C | LEU | A | 111 | 47.338 | 62.239 | 21.709 | 1.00 | 13.73 | A |
| ATOM | 686 | O | LEU | A | 111 | 47.777 | 61.158 | 22.113 | 1.00 | 14.06 | A |
| ATOM | 687 | N | SER | A | 112 | 47.573 | 63.388 | 22.332 | 1.00 | 14.19 | A |
| ATOM | 688 | CA | SER | A | 112 | 48.381 | 63.412 | 23.545 | 1.00 | 14.98 | A |
| ATOM | 689 | CB | SER | A | 112 | 48.673 | 64.852 | 23.965 | 1.00 | 15.68 | A |
| ATOM | 690 | OG | SER | A | 112 | 49.509 | 64.869 | 25.110 | 1.00 | 19.16 | A |
| ATOM | 691 | C | SER | A | 112 | 47.665 | 62.675 | 24.676 | 1.00 | 15.17 | A |
| ATOM | 692 | O | SER | A | 112 | 48.271 | 61.878 | 25.397 | 1.00 | 14.77 | A |
| ATOM | 693 | N | ASN | A | 113 | 46.371 | 62.935 | 24.833 | 1.00 | 14.95 | A |
| ATOM | 694 | CA | ASN | A | 113 | 45.623 | 62.269 | 25.887 | 1.00 | 15.42 | A |
| ATOM | 695 | CB | ASN | A | 113 | 44.348 | 63.055 | 26.202 | 1.00 | 16.09 | A |
| ATOM | 696 | CG | ASN | A | 113 | 44.661 | 64.407 | 26.829 | 1.00 | 17.13 | A |
| ATOM | 697 | OD1 | ASN | A | 113 | 45.649 | 64.541 | 27.547 | 1.00 | 19.22 | A |
| ATOM | 698 | ND2 | ASN | A | 113 | 43.832 | 65.404 | 26.566 | 1.00 | 16.69 | A |
| ATOM | 699 | C | ASN | A | 113 | 45.331 | 60.805 | 25.554 | 1.00 | 15.52 | A |
| ATOM | 700 | O | ASN | A | 113 | 45.154 | 59.983 | 26.455 | 1.00 | 14.44 | A |
| ATOM | 701 | N | ALA | A | 114 | 45.306 | 60.470 | 24.265 | 1.00 | 15.03 | A |
| ATOM | 702 | CA | ALA | A | 114 | 45.081 | 59.082 | 23.865 | 1.00 | 15.59 | A |
| ATOM | 703 | CB | ALA | A | 114 | 44.906 | 58.977 | 22.348 | 1.00 | 15.62 | A |
| ATOM | 704 | C | ALA | A | 114 | 46.296 | 58.272 | 24.308 | 1.00 | 15.90 | A |
| ATOM | 705 | O | ALA | A | 114 | 46.160 | 57.181 | 24.862 | 1.00 | 15.90 | A |
| ATOM | 706 | N | LEU | A | 115 | 47.487 | 58.814 | 24.061 | 1.00 | 15.81 | A |
| ATOM | 707 | CA | LEU | A | 115 | 48.725 | 58.142 | 24.443 | 1.00 | 16.69 | A |
| ATOM | 708 | CB | LEU | A | 115 | 49.942 | 58.992 | 24.044 | 1.00 | 17.24 | A |
| ATOM | 709 | CG | LEU | A | 115 | 51.322 | 58.478 | 24.479 | 1.00 | 17.48 | A |
| ATOM | 710 | CD1 | LEU | A | 115 | 51.533 | 57.062 | 23.962 | 1.00 | 17.85 | A |
| ATOM | 711 | CD2 | LEU | A | 115 | 52.412 | 59.402 | 23.963 | 1.00 | 17.89 | A |
| ATOM | 712 | C | LEU | A | 115 | 48.742 | 57.892 | 25.952 | 1.00 | 17.69 | A |
| ATOM | 713 | O | LEU | A | 115 | 49.020 | 56.780 | 26.407 | 1.00 | 16.58 | A |
| ATOM | 714 | N | ARG | A | 116 | 48.426 | 58.927 | 26.720 | 1.00 | 18.38 | A |
| ATOM | 715 | CA | ARG | A | 116 | 48.416 | 58.816 | 28.173 | 1.00 | 20.26 | A |
| ATOM | 716 | CB | ARG | A | 116 | 48.148 | 60.188 | 28.795 | 1.00 | 23.40 | A |
| ATOM | 717 | CG | ARG | A | 116 | 49.265 | 61.185 | 28.545 | 1.00 | 28.54 | A |
| ATOM | 718 | CD | ARG | A | 116 | 48.916 | 62.579 | 29.037 | 1.00 | 32.84 | A |
| ATOM | 719 | NE | ARG | A | 116 | 50.016 | 63.516 | 28.816 | 1.00 | 36.19 | A |
| ATOM | 720 | CZ | ARG | A | 116 | 49.955 | 64.819 | 29.078 | 1.00 | 38.16 | A |
| ATOM | 721 | NH1 | ARG | A | 116 | 48.842 | 65.347 | 29.572 | 1.00 | 39.38 | A |
| ATOM | 722 | NH2 | ARG | A | 116 | 51.009 | 65.594 | 28.851 | 1.00 | 39.35 | A |
| ATOM | 723 | C | ARG | A | 116 | 47.386 | 57.811 | 28.674 | 1.00 | 19.24 | A |
| ATOM | 724 | O | ARG | A | 116 | 47.713 | 56.895 | 29.433 | 1.00 | 18.59 | A |
| ATOM | 725 | N | HIS | A | 117 | 46.142 | 57.974 | 28.240 | 1.00 | 18.94 | A |
| ATOM | 726 | CA | HIS | A | 117 | 45.076 | 57.087 | 28.673 | 1.00 | 19.08 | A |
| ATOM | 727 | CB | HIS | A | 117 | 43.731 | 57.653 | 28.237 | 1.00 | 20.04 | A |
| ATOM | 728 | CG | HIS | A | 117 | 43.239 | 58.738 | 29.140 | 1.00 | 22.31 | A |
| ATOM | 729 | CD2 | HIS | A | 117 | 43.367 | 60.084 | 29.070 | 1.00 | 23.10 | A |
| ATOM | 730 | ND1 | HIS | A | 117 | 42.627 | 58.474 | 30.347 | 1.00 | 22.39 | A |
| ATOM | 731 | CE1 | HIS | A | 117 | 42.403 | 59.610 | 30.984 | 1.00 | 23.51 | A |
| ATOM | 732 | NE2 | HIS | A | 117 | 42.844 | 60.603 | 30.231 | 1.00 | 23.78 | A |
| ATOM | 733 | C | HIS | A | 117 | 45.208 | 55.630 | 28.267 | 1.00 | 18.21 | A |
| ATOM | 734 | O | HIS | A | 117 | 44.894 | 54.747 | 29.060 | 1.00 | 17.60 | A |
| ATOM | 735 | N | LEU | A | 118 | 45.667 | 55.367 | 27.047 | 1.00 | 17.82 | A |
| ATOM | 736 | CA | LEU | A | 118 | 45.841 | 53.986 | 26.608 | 1.00 | 17.73 | A |
| ATOM | 737 | CB | LEU | A | 118 | 46.097 | 53.924 | 25.097 | 1.00 | 17.32 | A |
| ATOM | 738 | CG | LEU | A | 118 | 44.910 | 54.378 | 24.234 | 1.00 | 17.44 | A |
| ATOM | 739 | CD1 | LEU | A | 118 | 45.295 | 54.362 | 22.762 | 1.00 | 17.28 | A |
| ATOM | 740 | CD2 | LEU | A | 118 | 43.719 | 53.466 | 24.485 | 1.00 | 17.73 | A |
| ATOM | 741 | C | LEU | A | 118 | 47.014 | 53.375 | 27.368 | 1.00 | 18.47 | A |
| ATOM | 742 | O | LEU | A | 118 | 46.991 | 52.201 | 27.739 | 1.00 | 19.15 | A |
| ATOM | 743 | N | HIS | A | 119 | 48.049 | 54.176 | 27.594 | 1.00 | 18.56 | A |
| ATOM | 744 | CA | HIS | A | 119 | 49.212 | 53.702 | 28.324 | 1.00 | 19.21 | A |
| ATOM | 745 | CB | HIS | A | 119 | 50.264 | 54.817 | 28.413 | 1.00 | 20.87 | A |
| ATOM | 746 | CG | HIS | A | 119 | 51.429 | 54.483 | 29.292 | 1.00 | 23.01 | A |
| ATOM | 747 | CD2 | HIS | A | 119 | 52.607 | 53.873 | 29.019 | 1.00 | 24.14 | A |
| ATOM | 748 | ND1 | HIS | A | 119 | 51.442 | 54.752 | 30.644 | 1.00 | 24.46 | A |
| ATOM | 749 | CE1 | HIS | A | 119 | 52.578 | 54.323 | 31.166 | 1.00 | 24.96 | A |
| ATOM | 750 | NE2 | HIS | A | 119 | 53.303 | 53.785 | 30.201 | 1.00 | 24.88 | A |
| ATOM | 751 | C | HIS | A | 119 | 48.795 | 53.248 | 29.725 | 1.00 | 19.46 | A |
| ATOM | 752 | O | HIS | A | 119 | 49.180 | 52.169 | 30.175 | 1.00 | 19.45 | A |
| ATOM | 753 | N | ASP | A | 120 | 47.981 | 54.063 | 30.392 | 1.00 | 18.91 | A |
| ATOM | 754 | CA | ASP | A | 120 | 47.524 | 53.772 | 31.751 | 1.00 | 19.04 | A |
| ATOM | 755 | CB | ASP | A | 120 | 47.142 | 55.071 | 32.464 | 1.00 | 18.77 | A |
| ATOM | 756 | CG | ASP | A | 120 | 48.325 | 55.994 | 32.666 | 1.00 | 19.47 | A |
| ATOM | 757 | OD1 | ASP | A | 120 | 49.473 | 55.504 | 32.675 | 1.00 | 18.88 | A |
| ATOM | 758 | OD2 | ASP | A | 120 | 48.102 | 57.212 | 32.830 | 1.00 | 21.58 | A |
| ATOM | 759 | C | ASP | A | 120 | 46.366 | 52.783 | 31.899 | 1.00 | 19.15 | A |
| ATOM | 760 | O | ASP | A | 120 | 46.121 | 52.286 | 32.998 | 1.00 | 18.84 | A |
| ATOM | 761 | N | ASN | A | 121 | 45.655 | 52.504 | 30.809 | 1.00 | 18.84 | A |
| ATOM | 762 | CA | ASN | A | 121 | 44.523 | 51.576 | 30.850 | 1.00 | 19.16 | A |
| ATOM | 763 | CB | ASN | A | 121 | 43.209 | 52.344 | 30.659 | 1.00 | 18.77 | A |
| ATOM | 764 | CG | ASN | A | 121 | 43.000 | 53.421 | 31.719 | 1.00 | 19.68 | A |
| ATOM | 765 | OD1 | ASN | A | 121 | 43.437 | 54.567 | 31.560 | 1.00 | 19.47 | A |
| ATOM | 766 | ND2 | ASN | A | 121 | 42.343 | 53.052 | 32.812 | 1.00 | 17.58 | A |
| ATOM | 767 | C | ASN | A | 121 | 44.681 | 50.515 | 29.761 | 1.00 | 19.99 | A |
| ATOM | 768 | O | ASN | A | 121 | 44.107 | 50.629 | 28.676 | 1.00 | 19.59 | A |
| ATOM | 769 | N | PRO | A | 122 | 45.452 | 49.452 | 30.052 | 1.00 | 20.58 | A |
| ATOM | 770 | CD | PRO | A | 122 | 45.971 | 49.147 | 31.397 | 1.00 | 21.14 | A |
| ATOM | 771 | CA | PRO | A | 122 | 45.731 | 48.345 | 29.130 | 1.00 | 20.26 | A |
| ATOM | 772 | CB | PRO | A | 122 | 46.423 | 47.310 | 30.026 | 1.00 | 21.39 | A |
| ATOM | 773 | CG | PRO | A | 122 | 45.933 | 47.649 | 31.409 | 1.00 | 22.22 | A |
| ATOM | 774 | C | PRO | A | 122 | 44.581 | 47.752 | 28.314 | 1.00 | 20.19 | A |
| ATOM | 775 | O | PRO | A | 122 | 44.802 | 47.304 | 27.188 | 1.00 | 19.85 | A |
| ATOM | 776 | N | GLU | A | 123 | 43.365 | 47.751 | 28.858 | 1.00 | 19.32 | A |
| ATOM | 777 | CA | GLU | A | 123 | 42.224 | 47.190 | 28.133 | 1.00 | 19.92 | A |
| ATOM | 778 | CB | GLU | A | 123 | 41.204 | 46.586 | 29.101 | 1.00 | 21.89 | A |
| ATOM | 779 | CG | GLU | A | 123 | 41.481 | 45.143 | 29.478 | 1.00 | 26.85 | A |
| ATOM | 780 | CD | GLU | A | 123 | 42.679 | 44.995 | 30.383 | 1.00 | 29.19 | A |
| ATOM | 781 | OE1 | GLU | A | 123 | 42.681 | 45.630 | 31.459 | 1.00 | 31.66 | A |
| ATOM | 782 | OE2 | GLU | A | 123 | 43.612 | 44.244 | 30.022 | 1.00 | 32.08 | A |
| ATOM | 783 | C | GLU | A | 123 | 41.504 | 48.168 | 27.213 | 1.00 | 18.82 | A |
| ATOM | 784 | O | GLU | A | 123 | 40.677 | 47.759 | 26.396 | 1.00 | 18.51 | A |
| ATOM | 785 | N | MET | A | 124 | 41.799 | 49.456 | 27.350 | 1.00 | 17.76 | A |
| ATOM | 786 | CA | MET | A | 124 | 41.165 | 50.462 | 26.505 | 1.00 | 17.02 | A |
| ATOM | 787 | CB | MET | A | 124 | 41.418 | 51.861 | 27.068 | 1.00 | 17.84 | A |
| ATOM | 788 | CG | MET | A | 124 | 40.641 | 52.961 | 26.357 | 1.00 | 17.84 | A |
| ATOM | 789 | SD | MET | A | 124 | 38.862 | 52.633 | 26.331 | 1.00 | 18.84 | A |
| ATOM | 790 | CE | MET | A | 124 | 38.252 | 54.142 | 25.567 | 1.00 | 17.66 | A |
| ATOM | 791 | C | MET | A | 124 | 41.744 | 50.351 | 25.092 | 1.00 | 16.66 | A |
| ATOM | 792 | O | MET | A | 124 | 42.921 | 50.022 | 24.922 | 1.00 | 15.61 | A |
| ATOM | 793 | N | LYS | A | 125 | 40.913 | 50.622 | 24.089 | 1.00 | 15.94 | A |
| ATOM | 794 | CA | LYS | A | 125 | 41.328 | 50.543 | 22.691 | 1.00 | 15.72 | A |
| ATOM | 795 | CB | LYS | A | 125 | 40.633 | 49.355 | 22.018 | 1.00 | 17.14 | A |
| ATOM | 796 | CG | LYS | A | 125 | 40.955 | 48.002 | 22.649 | 1.00 | 19.31 | A |
| ATOM | 797 | CD | LYS | A | 125 | 42.349 | 47.527 | 22.274 | 1.00 | 20.94 | A |
| ATOM | 798 | CE | LYS | A | 125 | 42.741 | 46.260 | 23.032 | 1.00 | 22.65 | A |
| ATOM | 799 | NZ | LYS | A | 125 | 41.809 | 45.126 | 22.787 | 1.00 | 22.71 | A |
| ATOM | 800 | C | LYS | A | 125 | 40.984 | 51.839 | 21.952 | 1.00 | 14.98 | A |
| ATOM | 801 | O | LYS | A | 125 | 40.178 | 52.641 | 22.430 | 1.00 | 14.40 | A |
| ATOM | 802 | N | PHE | A | 126 | 41.576 | 52.039 | 20.778 | 1.00 | 13.76 | A |
| ATOM | 803 | CA | PHE | A | 126 | 41.328 | 53.263 | 20.017 | 1.00 | 12.78 | A |
| ATOM | 804 | CB | PHE | A | 126 | 42.085 | 54.418 | 20.695 | 1.00 | 12.49 | A |
| ATOM | 805 | CG | PHE | A | 126 | 41.714 | 55.796 | 20.199 | 1.00 | 12.62 | A |
| ATOM | 806 | CD1 | PHE | A | 126 | 40.391 | 56.230 | 20.210 | 1.00 | 12.40 | A |
| ATOM | 807 | CD2 | PHE | A | 126 | 42.706 | 56.687 | 19.794 | 1.00 | 12.56 | A |
| ATOM | 808 | CE1 | PHE | A | 126 | 40.061 | 57.533 | 19.831 | 1.00 | 12.41 | A |
| ATOM | 809 | CE2 | PHE | A | 126 | 42.390 | 57.993 | 19.411 | 1.00 | 13.74 | A |
| ATOM | 810 | CZ | PHE | A | 126 | 41.063 | 58.418 | 19.431 | 1.00 | 12.79 | A |
| ATOM | 811 | C | PHE | A | 126 | 41.825 | 53.079 | 18.582 | 1.00 | 12.52 | A |
| ATOM | 812 | O | PHE | A | 126 | 42.898 | 52.523 | 18.365 | 1.00 | 12.34 | A |
| ATOM | 813 | N | ILE | A | 127 | 41.043 | 53.525 | 17.603 | 1.00 | 12.50 | A |
| ATOM | 814 | CA | ILE | A | 127 | 41.472 | 53.415 | 16.212 | 1.00 | 12.28 | A |
| ATOM | 815 | CB | ILE | A | 127 | 40.427 | 52.685 | 15.341 | 1.00 | 12.64 | A |
| ATOM | 816 | CG2 | ILE | A | 127 | 40.257 | 51.258 | 15.844 | 1.00 | 13.41 | A |
| ATOM | 817 | CG1 | ILE | A | 127 | 39.090 | 53.432 | 15.366 | 1.00 | 12.14 | A |
| ATOM | 818 | CD1 | ILE | A | 127 | 38.065 | 52.865 | 14.402 | 1.00 | 11.36 | A |
| ATOM | 819 | C | ILE | A | 127 | 41.735 | 54.806 | 15.640 | 1.00 | 12.00 | A |
| ATOM | 820 | O | ILE | A | 127 | 41.066 | 55.777 | 16.016 | 1.00 | 12.38 | A |
| ATOM | 821 | N | TRP | A | 128 | 42.720 | 54.905 | 14.749 | 1.00 | 11.18 | A |
| ATOM | 822 | CA | TRP | A | 128 | 43.067 | 56.187 | 14.137 | 1.00 | 10.75 | A |
| ATOM | 823 | CB | TRP | A | 128 | 44.379 | 56.714 | 14.713 | 1.00 | 10.60 | A |
| ATOM | 824 | CG | TRP | A | 128 | 44.614 | 58.143 | 14.353 | 1.00 | 11.47 | A |
| ATOM | 825 | CD2 | TRP | A | 128 | 44.052 | 59.285 | 15.004 | 1.00 | 11.70 | A |
| ATOM | 826 | CE2 | TRP | A | 128 | 44.492 | 60.427 | 14.298 | 1.00 | 11.23 | A |
| ATOM | 827 | CE3 | TRP | A | 128 | 43.214 | 59.455 | 16.117 | 1.00 | 11.54 | A |
| ATOM | 828 | CD1 | TRP | A | 128 | 45.353 | 58.620 | 13.307 | 1.00 | 12.07 | A |
| ATOM | 829 | NE1 | TRP | A | 128 | 45.285 | 59.995 | 13.268 | 1.00 | 11.96 | A |
| ATOM | 830 | CZ2 | TRP | A | 128 | 44.122 | 61.726 | 14.670 | 1.00 | 11.49 | A |
| ATOM | 831 | CZ3 | TRP | A | 128 | 42.847 | 60.747 | 16.484 | 1.00 | 11.63 | A |
| ATOM | 832 | CH2 | TRP | A | 128 | 43.302 | 61.865 | 15.761 | 1.00 | 11.38 | A |
| ATOM | 833 | C | TRP | A | 128 | 43.180 | 56.072 | 12.618 | 1.00 | 10.84 | A |
| ATOM | 834 | O | TRP | A | 128 | 43.820 | 55.157 | 12.102 | 1.00 | 9.94 | A |
| ATOM | 835 | N | ALA | A | 129 | 42.582 | 57.024 | 11.904 | 1.00 | 10.82 | A |
| ATOM | 836 | CA | ALA | A | 129 | 42.584 | 56.974 | 10.442 | 1.00 | 10.83 | A |
| ATOM | 837 | CB | ALA | A | 129 | 41.146 | 57.062 | 9.939 | 1.00 | 10.81 | A |
| ATOM | 838 | C | ALA | A | 129 | 43.439 | 57.982 | 9.675 | 1.00 | 11.28 | A |
| ATOM | 839 | O | ALA | A | 129 | 44.077 | 57.620 | 8.690 | 1.00 | 12.05 | A |
| ATOM | 840 | N | GLU | A | 130 | 43.450 | 59.234 | 10.122 | 1.00 | 11.63 | A |
| ATOM | 841 | CA | GLU | A | 130 | 44.178 | 60.298 | 9.426 | 1.00 | 11.80 | A |
| ATOM | 842 | CB | GLU | A | 130 | 43.488 | 61.640 | 9.687 | 1.00 | 13.00 | A |
| ATOM | 843 | CG | GLU | A | 130 | 41.996 | 61.654 | 9.375 | 1.00 | 13.47 | A |
| ATOM | 844 | CD | GLU | A | 130 | 41.150 | 61.054 | 10.488 | 1.00 | 14.76 | A |
| ATOM | 845 | OE1 | GLU | A | 130 | 41.706 | 60.745 | 11.564 | 1.00 | 13.75 | A |
| ATOM | 846 | OE2 | GLU | A | 130 | 39.925 | 60.903 | 10.289 | 1.00 | 14.95 | A |
| ATOM | 847 | C | GLU | A | 130 | 45.663 | 60.422 | 9.756 | 1.00 | 12.54 | A |
| ATOM | 848 | O | GLU | A | 130 | 46.044 | 61.043 | 10.751 | 1.00 | 11.25 | A |
| ATOM | 849 | N | ILE | A | 131 | 46.507 | 59.871 | 8.889 | 1.00 | 12.10 | A |
| ATOM | 850 | CA | ILE | A | 131 | 47.943 | 59.908 | 9.125 | 1.00 | 12.15 | A |
| ATOM | 851 | CB | ILE | A | 131 | 48.672 | 58.902 | 8.205 | 1.00 | 12.97 | A |
| ATOM | 852 | CG2 | ILE | A | 131 | 50.158 | 58.847 | 8.544 | 1.00 | 13.74 | A |
| ATOM | 853 | CG1 | ILE | A | 131 | 48.058 | 57.509 | 8.395 | 1.00 | 12.19 | A |
| ATOM | 854 | CD1 | ILE | A | 131 | 47.933 | 57.084 | 9.859 | 1.00 | 13.31 | A |
| ATOM | 855 | C | ILE | A | 131 | 48.564 | 61.303 | 9.002 | 1.00 | 12.69 | A |
| ATOM | 856 | O | ILE | A | 131 | 49.597 | 61.571 | 9.622 | 1.00 | 12.44 | A |
| ATOM | 857 | N | SER | A | 132 | 47.944 | 62.192 | 8.226 | 1.00 | 11.77 | A |
| ATOM | 858 | CA | SER | A | 132 | 48.462 | 63.553 | 8.087 | 1.00 | 12.31 | A |
| ATOM | 859 | CB | SER | A | 132 | 47.519 | 64.417 | 7.231 | 1.00 | 12.20 | A |
| ATOM | 860 | OG | SER | A | 132 | 46.188 | 64.405 | 7.729 | 1.00 | 12.12 | A |
| ATOM | 861 | C | SER | A | 132 | 48.606 | 64.161 | 9.485 | 1.00 | 12.41 | A |
| ATOM | 862 | O | SER | A | 132 | 49.629 | 64.772 | 9.809 | 1.00 | 11.98 | A |
| ATOM | 863 | N | TYR | A | 133 | 47.578 | 63.979 | 10.310 | 1.00 | 12.31 | A |
| ATOM | 864 | CA | TYR | A | 133 | 47.588 | 64.481 | 11.682 | 1.00 | 12.43 | A |
| ATOM | 865 | CB | TYR | A | 133 | 46.191 | 64.393 | 12.299 | 1.00 | 12.01 | A |
| ATOM | 866 | CG | TYR | A | 133 | 45.288 | 65.569 | 12.000 | 1.00 | 11.67 | A |
| ATOM | 867 | CD1 | TYR | A | 133 | 44.018 | 65.373 | 11.464 | 1.00 | 11.27 | A |
| ATOM | 868 | CE1 | TYR | A | 133 | 43.172 | 66.448 | 11.205 | 1.00 | 11.78 | A |
| ATOM | 869 | CD2 | TYR | A | 133 | 45.699 | 66.875 | 12.274 | 1.00 | 11.66 | A |
| ATOM | 870 | CE2 | TYR | A | 133 | 44.864 | 67.959 | 12.022 | 1.00 | 12.29 | A |
| ATOM | 871 | CZ | TYR | A | 133 | 43.603 | 67.738 | 11.488 | 1.00 | 12.81 | A |
| ATOM | 872 | OH | TYR | A | 133 | 42.772 | 68.809 | 11.248 | 1.00 | 13.96 | A |
| ATOM | 873 | C | TYR | A | 133 | 48.553 | 63.689 | 12.564 | 1.00 | 12.72 | A |
| ATOM | 874 | O | TYR | A | 133 | 49.314 | 64.275 | 13.339 | 1.00 | 13.12 | A |
| ATOM | 875 | N | PHE | A | 134 | 48.526 | 62.363 | 12.449 | 1.00 | 12.21 | A |
| ATOM | 876 | CA | PHE | A | 134 | 49.397 | 61.539 | 13.282 | 1.00 | 13.09 | A |
| ATOM | 877 | CB | PHE | A | 134 | 49.144 | 60.048 | 13.053 | 1.00 | 12.74 | A |
| ATOM | 878 | CG | PHE | A | 134 | 49.661 | 59.181 | 14.168 | 1.00 | 12.47 | A |
| ATOM | 879 | CD1 | PHE | A | 134 | 48.915 | 59.008 | 15.332 | 1.00 | 12.84 | A |
| ATOM | 880 | CD2 | PHE | A | 134 | 50.921 | 58.600 | 14.090 | 1.00 | 12.46 | A |
| ATOM | 881 | CE1 | PHE | A | 134 | 49.420 | 58.273 | 16.405 | 1.00 | 12.65 | A |
| ATOM | 882 | CE2 | PHE | A | 134 | 51.437 | 57.864 | 15.155 | 1.00 | 12.74 | A |
| ATOM | 883 | CZ | PHE | A | 134 | 50.684 | 57.702 | 16.318 | 1.00 | 12.65 | A |
| ATOM | 884 | C | PHE | A | 134 | 50.874 | 61.829 | 13.055 | 1.00 | 14.10 | A |
| ATOM | 885 | O | PHE | A | 134 | 51.655 | 61.896 | 14.009 | 1.00 | 13.54 | A |
| ATOM | 886 | N | ALA | A | 135 | 51.261 | 61.990 | 11.793 | 1.00 | 14.28 | A |
| ATOM | 887 | CA | ALA | A | 135 | 52.653 | 62.271 | 11.466 | 1.00 | 15.41 | A |
| ATOM | 888 | CB | ALA | A | 135 | 52.841 | 62.273 | 9.955 | 1.00 | 14.73 | A |
| ATOM | 889 | C | ALA | A | 135 | 53.065 | 63.619 | 12.062 | 1.00 | 16.07 | A |
| ATOM | 890 | O | ALA | A | 135 | 54.161 | 63.756 | 12.607 | 1.00 | 17.46 | A |
| ATOM | 891 | N | ARG | A | 136 | 52.178 | 64.604 | 11.954 | 1.00 | 15.92 | A |
| ATOM | 892 | CA | ARG | A | 136 | 52.413 | 65.947 | 12.487 | 1.00 | 17.41 | A |
| ATOM | 893 | CB | ARG | A | 136 | 51.188 | 66.829 | 12.215 | 1.00 | 18.09 | A |
| ATOM | 894 | CG | ARG | A | 136 | 51.225 | 68.223 | 12.856 | 1.00 | 19.04 | A |
| ATOM | 895 | CD | ARG | A | 136 | 51.950 | 69.249 | 11.986 | 1.00 | 20.59 | A |
| ATOM | 896 | NE | ARG | A | 136 | 51.870 | 70.596 | 12.557 | 1.00 | 20.53 | A |
| ATOM | 897 | CZ | ARG | A | 136 | 52.504 | 70.967 | 13.665 | 1.00 | 21.02 | A |
| ATOM | 898 | NH1 | ARG | A | 136 | 53.268 | 70.094 | 14.309 | 1.00 | 21.09 | A |
| ATOM | 899 | NH2 | ARG | A | 136 | 52.366 | 72.199 | 14.138 | 1.00 | 20.47 | A |
| ATOM | 900 | C | ARG | A | 136 | 52.659 | 65.866 | 13.997 | 1.00 | 17.71 | A |
| ATOM | 901 | O | ARG | A | 136 | 53.552 | 66.521 | 14.536 | 1.00 | 17.71 | A |
| ATOM | 902 | N | PHE | A | 137 | 51.856 | 65.045 | 14.666 | 1.00 | 16.73 | A |
| ATOM | 903 | CA | PHE | A | 137 | 51.948 | 64.856 | 16.109 | 1.00 | 16.04 | A |
| ATOM | 904 | CB | PHE | A | 137 | 50.730 | 64.065 | 16.589 | 1.00 | 16.01 | A |
| ATOM | 905 | CG | PHE | A | 137 | 50.711 | 63.815 | 18.066 | 1.00 | 16.01 | A |
| ATOM | 906 | CD1 | PHE | A | 137 | 50.393 | 64.839 | 18.952 | 1.00 | 16.16 | A |
| ATOM | 907 | CD2 | PHE | A | 137 | 51.009 | 62.553 | 18.572 | 1.00 | 16.49 | A |
| ATOM | 908 | CE1 | PHE | A | 137 | 50.370 | 64.611 | 20.323 | 1.00 | 16.36 | A |
| ATOM | 909 | CE2 | PHE | A | 137 | 50.989 | 62.316 | 19.947 | 1.00 | 16.39 | A |
| ATOM | 910 | CZ | PHE | A | 137 | 50.667 | 63.349 | 20.821 | 1.00 | 16.41 | A |
| ATOM | 911 | C | PHE | A | 137 | 53.218 | 64.115 | 16.518 | 1.00 | 16.50 | A |
| ATOM | 912 | O | PHE | A | 137 | 54.012 | 64.600 | 17.329 | 1.00 | 15.50 | A |
| ATOM | 913 | N | TYR | A | 138 | 53.398 | 62.930 | 15.944 | 1.00 | 16.96 | A |
| ATOM | 914 | CA | TYR | A | 138 | 54.544 | 62.084 | 16.243 | 1.00 | 19.17 | A |
| ATOM | 915 | CB | TYR | A | 138 | 54.547 | 60.866 | 15.323 | 1.00 | 18.81 | A |
| ATOM | 916 | CG | TYR | A | 138 | 55.577 | 59.830 | 15.706 | 1.00 | 19.98 | A |
| ATOM | 917 | CD1 | TYR | A | 138 | 55.330 | 58.927 | 16.736 | 1.00 | 20.01 | A |
| ATOM | 918 | CE1 | TYR | A | 138 | 56.272 | 57.965 | 17.093 | 1.00 | 21.20 | A |
| ATOM | 919 | CD2 | TYR | A | 138 | 56.801 | 59.751 | 15.040 | 1.00 | 19.71 | A |
| ATOM | 920 | CE2 | TYR | A | 138 | 57.752 | 58.791 | 15.390 | 1.00 | 20.51 | A |
| ATOM | 921 | CZ | TYR | A | 138 | 57.477 | 57.902 | 16.414 | 1.00 | 20.46 | A |
| ATOM | 922 | OH | TYR | A | 138 | 58.393 | 56.934 | 16.753 | 1.00 | 21.98 | A |
| ATOM | 923 | C | TYR | A | 138 | 55.895 | 62.788 | 16.137 | 1.00 | 20.16 | A |
| ATOM | 924 | O | TYR | A | 138 | 56.737 | 62.662 | 17.030 | 1.00 | 19.95 | A |
| ATOM | 925 | N | HIS | A | 139 | 56.116 | 63.516 | 15.047 | 1.00 | 21.15 | A |
| ATOM | 926 | CA | HIS | A | 139 | 57.391 | 64.206 | 14.870 | 1.00 | 23.05 | A |
| ATOM | 927 | CB | HIS | A | 139 | 57.491 | 64.788 | 13.459 | 1.00 | 23.52 | A |
| ATOM | 928 | CG | HIS | A | 139 | 57.664 | 63.749 | 12.394 | 1.00 | 25.29 | A |
| ATOM | 929 | CD2 | HIS | A | 139 | 56.872 | 63.396 | 11.353 | 1.00 | 25.23 | A |
| ATOM | 930 | ND1 | HIS | A | 139 | 58.766 | 62.922 | 12.333 | 1.00 | 26.05 | A |
| ATOM | 931 | CE1 | HIS | A | 139 | 58.645 | 62.106 | 11.301 | 1.00 | 26.57 | A |
| ATOM | 932 | NE2 | HIS | A | 139 | 57.505 | 62.373 | 10.690 | 1.00 | 25.58 | A |
| ATOM | 933 | C | HIS | A | 139 | 57.628 | 65.297 | 15.910 | 1.00 | 23.20 | A |
| ATOM | 934 | O | HIS | A | 139 | 58.763 | 65.722 | 16.121 | 1.00 | 23.77 | A |
| ATOM | 935 | N | ASP | A | 140 | 56.559 | 65.743 | 16.560 | 1.00 | 23.11 | A |
| ATOM | 936 | CA | ASP | A | 140 | 56.663 | 66.772 | 17.590 | 1.00 | 23.35 | A |
| ATOM | 937 | CB | ASP | A | 140 | 55.405 | 67.644 | 17.591 | 1.00 | 24.33 | A |
| ATOM | 938 | CG | ASP | A | 140 | 55.514 | 68.827 | 16.646 | 1.00 | 26.10 | A |
| ATOM | 939 | OD1 | ASP | A | 140 | 56.367 | 68.786 | 15.734 | 1.00 | 26.58 | A |
| ATOM | 940 | OD2 | ASP | A | 140 | 54.742 | 69.796 | 16.812 | 1.00 | 26.82 | A |
| ATOM | 941 | C | ASP | A | 140 | 56.879 | 66.164 | 18.980 | 1.00 | 22.72 | A |
| ATOM | 942 | O | ASP | A | 140 | 57.232 | 66.868 | 19.925 | 1.00 | 22.14 | A |
| ATOM | 943 | N | LEU | A | 141 | 56.674 | 64.855 | 19.094 | 1.00 | 21.94 | A |
| ATOM | 944 | CA | LEU | A | 141 | 56.850 | 64.155 | 20.366 | 1.00 | 22.28 | A |
| ATOM | 945 | CB | LEU | A | 141 | 56.191 | 62.774 | 20.322 | 1.00 | 22.07 | A |
| ATOM | 946 | CG | LEU | A | 141 | 54.673 | 62.622 | 20.389 | 1.00 | 22.20 | A |
| ATOM | 947 | CD1 | LEU | A | 141 | 54.324 | 61.135 | 20.279 | 1.00 | 21.62 | A |
| ATOM | 948 | CD2 | LEU | A | 141 | 54.144 | 63.195 | 21.695 | 1.00 | 21.60 | A |
| ATOM | 949 | C | LEU | A | 141 | 58.308 | 63.959 | 20.747 | 1.00 | 22.47 | A |
| ATOM | 950 | O | LEU | A | 141 | 59.178 | 63.846 | 19.886 | 1.00 | 21.96 | A |
| ATOM | 951 | N | GLY | A | 142 | 58.567 | 63.916 | 22.050 | 1.00 | 23.34 | A |
| ATOM | 952 | CA | GLY | A | 142 | 59.918 | 63.687 | 22.524 | 1.00 | 24.09 | A |
| ATOM | 953 | C | GLY | A | 142 | 60.200 | 62.205 | 22.356 | 1.00 | 24.71 | A |
| ATOM | 954 | O | GLY | A | 142 | 59.268 | 61.410 | 22.220 | 1.00 | 24.43 | A |
| ATOM | 955 | N | GLU | A | 143 | 61.473 | 61.826 | 22.371 | 1.00 | 25.38 | A |
| ATOM | 956 | CA | GLU | A | 143 | 61.861 | 60.432 | 22.196 | 1.00 | 26.27 | A |
| ATOM | 957 | CB | GLU | A | 143 | 63.379 | 60.292 | 22.323 | 1.00 | 27.58 | A |
| ATOM | 958 | CG | GLU | A | 143 | 63.922 | 58.940 | 21.884 | 1.00 | 29.88 | A |
| ATOM | 959 | CD | GLU | A | 143 | 63.538 | 58.590 | 20.455 | 1.00 | 31.60 | A |
| ATOM | 960 | OE1 | GLU | A | 143 | 63.736 | 59.433 | 19.555 | 1.00 | 33.17 | A |
| ATOM | 961 | OE2 | GLU | A | 143 | 63.042 | 57.467 | 20.230 | 1.00 | 32.74 | A |
| ATOM | 962 | C | GLU | A | 143 | 61.170 | 59.477 | 23.166 | 1.00 | 26.12 | A |
| ATOM | 963 | O | GLU | A | 143 | 60.738 | 58.394 | 22.772 | 1.00 | 25.94 | A |
| ATOM | 964 | N | ASN | A | 144 | 61.067 | 59.871 | 24.430 | 1.00 | 26.24 | A |
| ATOM | 965 | CA | ASN | A | 144 | 60.421 | 59.028 | 25.431 | 1.00 | 26.68 | A |
| ATOM | 966 | CB | ASN | A | 144 | 60.460 | 59.713 | 26.806 | 1.00 | 28.10 | A |
| ATOM | 967 | CG | ASN | A | 144 | 59.674 | 58.953 | 27.866 | 1.00 | 30.54 | A |
| ATOM | 968 | OD1 | ASN | A | 144 | 58.439 | 58.938 | 27.854 | 1.00 | 31.68 | A |
| ATOM | 969 | ND2 | ASN | A | 144 | 60.390 | 58.316 | 28.791 | 1.00 | 31.00 | A |
| ATOM | 970 | C | ASN | A | 144 | 58.977 | 58.725 | 25.031 | 1.00 | 25.49 | A |
| ATOM | 971 | O | ASN | A | 144 | 58.539 | 57.575 | 25.089 | 1.00 | 25.02 | A |
| ATOM | 972 | N | LYS | A | 145 | 58.249 | 59.757 | 24.613 | 1.00 | 24.88 | A |
| ATOM | 973 | CA | LYS | A | 145 | 56.855 | 59.595 | 24.205 | 1.00 | 24.21 | A |
| ATOM | 974 | CB | LYS | A | 145 | 56.180 | 60.964 | 24.072 | 1.00 | 24.66 | A |
| ATOM | 975 | CG | LYS | A | 145 | 55.898 | 61.642 | 25.410 | 1.00 | 26.34 | A |
| ATOM | 976 | CD | LYS | A | 145 | 54.937 | 60.805 | 26.245 | 1.00 | 27.93 | A |
| ATOM | 977 | CE | LYS | A | 145 | 54.660 | 61.433 | 27.608 | 1.00 | 29.60 | A |
| ATOM | 978 | NZ | LYS | A | 145 | 55.876 | 61.471 | 28.469 | 1.00 | 31.08 | A |
| ATOM | 979 | C | LYS | A | 145 | 56.705 | 58.804 | 22.907 | 1.00 | 23.37 | A |
| ATOM | 980 | O | LYS | A | 145 | 55.740 | 58.055 | 22.745 | 1.00 | 22.99 | A |
| ATOM | 981 | N | LYS | A | 146 | 57.648 | 58.968 | 21.981 | 1.00 | 22.74 | A |
| ATOM | 982 | CA | LYS | A | 146 | 57.594 | 58.226 | 20.724 | 1.00 | 21.93 | A |
| ATOM | 983 | CB | LYS | A | 146 | 58.764 | 58.599 | 19.804 | 1.00 | 22.03 | A |
| ATOM | 984 | CG | LYS | A | 146 | 58.632 | 59.956 | 19.126 | 1.00 | 22.74 | A |
| ATOM | 985 | CD | LYS | A | 146 | 59.765 | 60.194 | 18.136 | 1.00 | 23.91 | A |
| ATOM | 986 | CE | LYS | A | 146 | 59.607 | 61.537 | 17.437 | 1.00 | 24.86 | A |
| ATOM | 987 | NZ | LYS | A | 146 | 60.724 | 61.791 | 16.495 | 1.00 | 26.86 | A |
| ATOM | 988 | C | LYS | A | 146 | 57.652 | 56.735 | 21.034 | 1.00 | 21.31 | A |
| ATOM | 989 | O | LYS | A | 146 | 56.973 | 55.935 | 20.396 | 1.00 | 20.71 | A |
| ATOM | 990 | N | LEU | A | 147 | 58.466 | 56.371 | 22.022 | 1.00 | 20.89 | A |
| ATOM | 991 | CA | LEU | A | 147 | 58.612 | 54.978 | 22.427 | 1.00 | 20.24 | A |
| ATOM | 992 | CB | LEU | A | 147 | 59.790 | 54.829 | 23.396 | 1.00 | 21.06 | A |
| ATOM | 993 | CG | LEU | A | 147 | 61.170 | 55.069 | 22.769 | 1.00 | 21.23 | A |
| ATOM | 994 | CD1 | LEU | A | 147 | 62.262 | 54.965 | 23.827 | 1.00 | 22.02 | A |
| ATOM | 995 | CD2 | LEU | A | 147 | 61.405 | 54.044 | 21.668 | 1.00 | 22.53 | A |
| ATOM | 996 | C | LEU | A | 147 | 57.325 | 54.455 | 23.067 | 1.00 | 20.09 | A |
| ATOM | 997 | O | LEU | A | 147 | 56.907 | 53.330 | 22.796 | 1.00 | 19.03 | A |
| ATOM | 998 | N | GLN | A | 148 | 56.699 | 55.260 | 23.920 | 1.00 | 20.05 | A |
| ATOM | 999 | CA | GLN | A | 148 | 55.448 | 54.839 | 24.541 | 1.00 | 20.20 | A |
| ATOM | 1000 | CB | GLN | A | 148 | 54.951 | 55.874 | 25.553 | 1.00 | 21.98 | A |
| ATOM | 1001 | CG | GLN | A | 148 | 55.697 | 55.876 | 26.873 | 1.00 | 26.14 | A |
| ATOM | 1002 | CD | GLN | A | 148 | 55.039 | 56.771 | 27.910 | 1.00 | 28.69 | A |
| ATOM | 1003 | OE1 | GLN | A | 148 | 55.477 | 56.830 | 29.061 | 1.00 | 30.91 | A |
| ATOM | 1004 | NE2 | GLN | A | 148 | 53.980 | 57.472 | 27.507 | 1.00 | 28.81 | A |
| ATOM | 1005 | C | GLN | A | 148 | 54.387 | 54.657 | 23.459 | 1.00 | 18.95 | A |
| ATOM | 1006 | O | GLN | A | 148 | 53.566 | 53.743 | 23.527 | 1.00 | 18.96 | A |
| ATOM | 1007 | N | MET | A | 149 | 54.410 | 55.533 | 22.462 | 1.00 | 17.60 | A |
| ATOM | 1008 | CA | MET | A | 149 | 53.440 | 55.462 | 21.376 | 1.00 | 16.91 | A |
| ATOM | 1009 | CB | MET | A | 149 | 53.563 | 56.690 | 20.471 | 1.00 | 17.32 | A |
| ATOM | 1010 | CG | MET | A | 149 | 52.539 | 56.743 | 19.344 | 1.00 | 17.23 | A |
| ATOM | 1011 | SD | MET | A | 149 | 50.830 | 56.820 | 19.927 | 1.00 | 19.29 | A |
| ATOM | 1012 | CE | MET | A | 149 | 50.590 | 58.587 | 20.083 | 1.00 | 18.52 | A |
| ATOM | 1013 | C | MET | A | 149 | 53.641 | 54.190 | 20.558 | 1.00 | 16.89 | A |
| ATOM | 1014 | O | MET | A | 149 | 52.680 | 53.502 | 20.226 | 1.00 | 14.92 | A |
| ATOM | 1015 | N | LYS | A | 150 | 54.892 | 53.872 | 20.239 | 1.00 | 17.09 | A |
| ATOM | 1016 | CA | LYS | A | 150 | 55.171 | 52.675 | 19.458 | 1.00 | 18.24 | A |
| ATOM | 1017 | CB | LYS | A | 150 | 56.660 | 52.593 | 19.106 | 1.00 | 19.09 | A |
| ATOM | 1018 | CG | LYS | A | 150 | 57.130 | 53.715 | 18.192 | 1.00 | 22.02 | A |
| ATOM | 1019 | CD | LYS | A | 150 | 58.638 | 53.704 | 17.997 | 1.00 | 24.66 | A |
| ATOM | 1020 | CE | LYS | A | 150 | 59.093 | 52.476 | 17.234 | 1.00 | 26.81 | A |
| ATOM | 1021 | NZ | LYS | A | 150 | 60.558 | 52.520 | 16.961 | 1.00 | 29.58 | A |
| ATOM | 1022 | C | LYS | A | 150 | 54.745 | 51.422 | 20.209 | 1.00 | 18.20 | A |
| ATOM | 1023 | O | LYS | A | 150 | 54.317 | 50.446 | 19.597 | 1.00 | 18.07 | A |
| ATOM | 1024 | N | SER | A | 151 | 54.843 | 51.455 | 21.536 | 1.00 | 18.74 | A |
| ATOM | 1025 | CA | SER | A | 151 | 54.474 | 50.295 | 22.339 | 1.00 | 19.47 | A |
| ATOM | 1026 | CB | SER | A | 151 | 55.005 | 50.440 | 23.770 | 1.00 | 21.09 | A |
| ATOM | 1027 | OG | SER | A | 151 | 54.283 | 51.425 | 24.481 | 1.00 | 24.31 | A |
| ATOM | 1028 | C | SER | A | 151 | 52.968 | 50.029 | 22.371 | 1.00 | 19.31 | A |
| ATOM | 1029 | O | SER | A | 151 | 52.547 | 48.875 | 22.278 | 1.00 | 18.16 | A |
| ATOM | 1030 | N | ILE | A | 152 | 52.150 | 51.073 | 22.499 | 1.00 | 18.86 | A |
| ATOM | 1031 | CA | ILE | A | 152 | 50.710 | 50.848 | 22.526 | 1.00 | 18.53 | A |
| ATOM | 1032 | CB | ILE | A | 152 | 49.926 | 52.069 | 23.075 | 1.00 | 18.94 | A |
| ATOM | 1033 | CG2 | ILE | A | 152 | 50.259 | 52.272 | 24.547 | 1.00 | 19.11 | A |
| ATOM | 1034 | CG1 | ILE | A | 152 | 50.243 | 53.328 | 22.272 | 1.00 | 18.05 | A |
| ATOM | 1035 | CD1 | ILE | A | 152 | 49.361 | 54.503 | 22.647 | 1.00 | 19.59 | A |
| ATOM | 1036 | C | ILE | A | 152 | 50.176 | 50.464 | 21.146 | 1.00 | 18.93 | A |
| ATOM | 1037 | O | ILE | A | 152 | 49.071 | 49.940 | 21.026 | 1.00 | 18.92 | A |
| ATOM | 1038 | N | VAL | A | 153 | 50.962 | 50.720 | 20.105 | 1.00 | 18.77 | A |
| ATOM | 1039 | CA | VAL | A | 153 | 50.561 | 50.341 | 18.752 | 1.00 | 19.06 | A |
| ATOM | 1040 | CB | VAL | A | 153 | 51.279 | 51.193 | 17.683 | 1.00 | 19.26 | A |
| ATOM | 1041 | CG1 | VAL | A | 153 | 51.069 | 50.589 | 16.295 | 1.00 | 18.53 | A |
| ATOM | 1042 | CG2 | VAL | A | 153 | 50.745 | 52.617 | 17.716 | 1.00 | 18.92 | A |
| ATOM | 1043 | C | VAL | A | 153 | 50.959 | 48.878 | 18.580 | 1.00 | 19.85 | A |
| ATOM | 1044 | O | VAL | A | 153 | 50.177 | 48.053 | 18.108 | 1.00 | 18.89 | A |
| ATOM | 1045 | N | LYS | A | 154 | 52.183 | 48.564 | 18.993 | 1.00 | 20.46 | A |
| ATOM | 1046 | CA | LYS | A | 154 | 52.696 | 47.205 | 18.893 | 1.00 | 22.15 | A |
| ATOM | 1047 | CB | LYS | A | 154 | 54.143 | 47.155 | 19.397 | 1.00 | 23.38 | A |
| ATOM | 1048 | CG | LYS | A | 154 | 54.885 | 45.877 | 19.026 | 1.00 | 26.39 | A |
| ATOM | 1049 | CD | LYS | A | 154 | 56.375 | 45.976 | 19.337 | 1.00 | 28.38 | A |
| ATOM | 1050 | CE | LYS | A | 154 | 56.662 | 45.794 | 20.822 | 1.00 | 30.74 | A |
| ATOM | 1051 | NZ | LYS | A | 154 | 55.974 | 46.793 | 21.694 | 1.00 | 32.22 | A |
| ATOM | 1052 | C | LYS | A | 154 | 51.836 | 46.218 | 19.685 | 1.00 | 22.01 | A |
| ATOM | 1053 | O | LYS | A | 154 | 51.608 | 45.090 | 19.236 | 1.00 | 22.53 | A |
| ATOM | 1054 | N | ASN | A | 155 | 51.349 | 46.642 | 20.852 | 1.00 | 21.36 | A |
| ATOM | 1055 | CA | ASN | A | 155 | 50.529 | 45.771 | 21.691 | 1.00 | 21.36 | A |
| ATOM | 1056 | CB | ASN | A | 155 | 50.643 | 46.174 | 23.172 | 1.00 | 22.78 | A |
| ATOM | 1057 | CG | ASN | A | 155 | 49.845 | 47.425 | 23.515 | 1.00 | 23.55 | A |
| ATOM | 1058 | OD1 | ASN | A | 155 | 49.106 | 47.954 | 22.687 | 1.00 | 24.96 | A |
| ATOM | 1059 | ND2 | ASN | A | 155 | 49.987 | 47.898 | 24.753 | 1.00 | 22.79 | A |
| ATOM | 1060 | C | ASN | A | 155 | 49.054 | 45.709 | 21.285 | 1.00 | 20.96 | A |
| ATOM | 1061 | O | ASN | A | 155 | 48.271 | 44.991 | 21.899 | 1.00 | 20.84 | A |
| ATOM | 1062 | N | GLY | A | 156 | 48.672 | 46.476 | 20.267 | 1.00 | 20.22 | A |
| ATOM | 1063 | CA | GLY | A | 156 | 47.298 | 46.431 | 19.790 | 1.00 | 19.12 | A |
| ATOM | 1064 | C | GLY | A | 156 | 46.256 | 47.367 | 20.378 | 1.00 | 18.60 | A |
| ATOM | 1065 | O | GLY | A | 156 | 45.082 | 47.270 | 20.011 | 1.00 | 18.18 | A |
| ATOM | 1066 | N | GLN | A | 157 | 46.652 | 48.266 | 21.276 | 1.00 | 17.29 | A |
| ATOM | 1067 | CA | GLN | A | 157 | 45.689 | 49.195 | 21.871 | 1.00 | 16.05 | A |
| ATOM | 1068 | CB | GLN | A | 157 | 46.242 | 49.809 | 23.150 | 1.00 | 16.21 | A |
| ATOM | 1069 | CG | GLN | A | 157 | 46.297 | 48.867 | 24.333 | 1.00 | 16.29 | A |
| ATOM | 1070 | CD | GLN | A | 157 | 46.569 | 49.621 | 25.612 | 1.00 | 16.60 | A |
| ATOM | 1071 | OE1 | GLN | A | 157 | 45.672 | 50.249 | 26.178 | 1.00 | 18.00 | A |
| ATOM | 1072 | NE2 | GLN | A | 157 | 47.813 | 49.591 | 26.060 | 1.00 | 15.39 | A |
| ATOM | 1073 | C | GLN | A | 157 | 45.305 | 50.320 | 20.917 | 1.00 | 15.53 | A |
| ATOM | 1074 | O | GLN | A | 157 | 44.142 | 50.700 | 20.832 | 1.00 | 15.19 | A |
| ATOM | 1075 | N | LEU | A | 158 | 46.298 | 50.881 | 20.235 | 1.00 | 14.97 | A |
| ATOM | 1076 | CA | LEU | A | 158 | 46.048 | 51.937 | 19.265 | 1.00 | 15.35 | A |
| ATOM | 1077 | CB | LEU | A | 158 | 47.045 | 53.089 | 19.439 | 1.00 | 16.96 | A |
| ATOM | 1078 | CG | LEU | A | 158 | 46.943 | 54.280 | 18.473 | 1.00 | 19.11 | A |
| ATOM | 1079 | CD1 | LEU | A | 158 | 47.454 | 53.891 | 17.096 | 1.00 | 21.08 | A |
| ATOM | 1080 | CD2 | LEU | A | 158 | 45.509 | 54.763 | 18.388 | 1.00 | 20.07 | A |
| ATOM | 1081 | C | LEU | A | 158 | 46.229 | 51.276 | 17.907 | 1.00 | 14.83 | A |
| ATOM | 1082 | O | LEU | A | 158 | 47.308 | 50.773 | 17.594 | 1.00 | 13.42 | A |
| ATOM | 1083 | N | GLU | A | 159 | 45.169 | 51.273 | 17.107 | 1.00 | 13.60 | A |
| ATOM | 1084 | CA | GLU | A | 159 | 45.227 | 50.636 | 15.802 | 1.00 | 13.04 | A |
| ATOM | 1085 | CB | GLU | A | 159 | 44.271 | 49.442 | 15.771 | 1.00 | 13.44 | A |
| ATOM | 1086 | CG | GLU | A | 159 | 44.212 | 48.714 | 14.436 | 1.00 | 12.38 | A |
| ATOM | 1087 | CD | GLU | A | 159 | 43.265 | 47.531 | 14.476 | 1.00 | 13.56 | A |
| ATOM | 1088 | OE1 | GLU | A | 159 | 43.594 | 46.533 | 15.152 | 1.00 | 12.89 | A |
| ATOM | 1089 | OE2 | GLU | A | 159 | 42.190 | 47.603 | 13.841 | 1.00 | 13.05 | A |
| ATOM | 1090 | C | GLU | A | 159 | 44.888 | 51.594 | 14.671 | 1.00 | 12.84 | A |
| ATOM | 1091 | O | GLU | A | 159 | 43.910 | 52.333 | 14.739 | 1.00 | 12.78 | A |
| ATOM | 1092 | N | PHE | A | 160 | 45.709 | 51.578 | 13.632 | 1.00 | 12.42 | A |
| ATOM | 1093 | CA | PHE | A | 160 | 45.467 | 52.437 | 12.491 | 1.00 | 12.26 | A |
| ATOM | 1094 | CB | PHE | A | 160 | 46.782 | 52.808 | 11.807 | 1.00 | 11.78 | A |
| ATOM | 1095 | CG | PHE | A | 160 | 47.712 | 53.586 | 12.689 | 1.00 | 12.41 | A |
| ATOM | 1096 | CD1 | PHE | A | 160 | 48.758 | 52.951 | 13.354 | 1.00 | 12.62 | A |
| ATOM | 1097 | CD2 | PHE | A | 160 | 47.512 | 54.947 | 12.897 | 1.00 | 12.79 | A |
| ATOM | 1098 | CE1 | PHE | A | 160 | 49.589 | 53.661 | 14.216 | 1.00 | 12.69 | A |
| ATOM | 1099 | CE2 | PHE | A | 160 | 48.339 | 55.666 | 13.760 | 1.00 | 13.12 | A |
| ATOM | 1100 | CZ | PHE | A | 160 | 49.375 | 55.020 | 14.418 | 1.00 | 12.71 | A |
| ATOM | 1101 | C | PHE | A | 160 | 44.546 | 51.734 | 11.515 | 1.00 | 12.26 | A |
| ATOM | 1102 | O | PHE | A | 160 | 44.719 | 50.549 | 11.216 | 1.00 | 11.96 | A |
| ATOM | 1103 | N | VAL | A | 161 | 43.546 | 52.470 | 11.047 | 1.00 | 12.03 | A |
| ATOM | 1104 | CA | VAL | A | 161 | 42.592 | 51.945 | 10.087 | 1.00 | 11.93 | A |
| ATOM | 1105 | CB | VAL | A | 161 | 41.139 | 52.092 | 10.605 | 1.00 | 11.85 | A |
| ATOM | 1106 | CG1 | VAL | A | 161 | 40.918 | 51.135 | 11.778 | 1.00 | 11.73 | A |
| ATOM | 1107 | CG2 | VAL | A | 161 | 40.874 | 53.526 | 11.059 | 1.00 | 11.37 | A |
| ATOM | 1108 | C | VAL | A | 161 | 42.807 | 52.720 | 8.792 | 1.00 | 12.32 | A |
| ATOM | 1109 | O | VAL | A | 161 | 42.890 | 53.953 | 8.802 | 1.00 | 12.53 | A |
| ATOM | 1110 | N | THR | A | 162 | 42.913 | 51.977 | 7.690 | 1.00 | 12.37 | A |
| ATOM | 1111 | CA | THR | A | 162 | 43.179 | 52.519 | 6.358 | 1.00 | 12.07 | A |
| ATOM | 1112 | CB | THR | A | 162 | 42.266 | 53.716 | 5.990 | 1.00 | 11.93 | A |
| ATOM | 1113 | OG1 | THR | A | 162 | 40.893 | 53.308 | 6.020 | 1.00 | 11.88 | A |
| ATOM | 1114 | CG2 | THR | A | 162 | 42.591 | 54.204 | 4.576 | 1.00 | 11.95 | A |
| ATOM | 1115 | C | THR | A | 162 | 44.640 | 52.971 | 6.310 | 1.00 | 12.30 | A |
| ATOM | 1116 | O | THR | A | 162 | 45.448 | 52.416 | 5.565 | 1.00 | 12.72 | A |
| ATOM | 1117 | N | GLY | A | 163 | 44.981 | 53.973 | 7.112 | 1.00 | 11.76 | A |
| ATOM | 1118 | CA | GLY | A | 163 | 46.356 | 54.444 | 7.144 | 1.00 | 11.74 | A |
| ATOM | 1119 | C | GLY | A | 163 | 46.722 | 55.405 | 6.032 | 1.00 | 12.16 | A |
| ATOM | 1120 | O | GLY | A | 163 | 47.895 | 55.554 | 5.698 | 1.00 | 11.80 | A |
| ATOM | 1121 | N | GLY | A | 164 | 45.718 | 56.049 | 5.449 | 1.00 | 11.89 | A |
| ATOM | 1122 | CA | GLY | A | 164 | 45.979 | 57.012 | 4.397 | 1.00 | 11.02 | A |
| ATOM | 1123 | C | GLY | A | 164 | 46.273 | 58.376 | 4.995 | 1.00 | 10.65 | A |
| ATOM | 1124 | O | GLY | A | 164 | 45.990 | 58.626 | 6.169 | 1.00 | 10.03 | A |
| ATOM | 1125 | N | TRP | A | 165 | 46.849 | 59.263 | 4.192 | 1.00 | 10.36 | A |
| ATOM | 1126 | CA | TRP | A | 165 | 47.163 | 60.607 | 4.662 | 1.00 | 10.35 | A |
| ATOM | 1127 | CB | TRP | A | 165 | 47.748 | 61.432 | 3.511 | 1.00 | 10.07 | A |
| ATOM | 1128 | CG | TRP | A | 165 | 48.536 | 62.640 | 3.941 | 1.00 | 11.12 | A |
| ATOM | 1129 | CD2 | TRP | A | 165 | 49.723 | 62.652 | 4.747 | 1.00 | 10.98 | A |
| ATOM | 1130 | CE2 | TRP | A | 165 | 50.142 | 63.999 | 4.855 | 1.00 | 11.20 | A |
| ATOM | 1131 | CE3 | TRP | A | 165 | 50.474 | 61.657 | 5.385 | 1.00 | 11.21 | A |
| ATOM | 1132 | CD1 | TRP | A | 165 | 48.291 | 63.943 | 3.605 | 1.00 | 10.95 | A |
| ATOM | 1133 | NE1 | TRP | A | 165 | 49.251 | 64.764 | 4.151 | 1.00 | 11.36 | A |
| ATOM | 1134 | CZ2 | TRP | A | 165 | 51.282 | 64.378 | 5.580 | 1.00 | 11.19 | A |
| ATOM | 1135 | CZ3 | TRP | A | 165 | 51.611 | 62.036 | 6.106 | 1.00 | 12.25 | A |
| ATOM | 1136 | CH2 | TRP | A | 165 | 52.000 | 63.384 | 6.194 | 1.00 | 11.49 | A |
| ATOM | 1137 | C | TRP | A | 165 | 45.852 | 61.224 | 5.157 | 1.00 | 10.30 | A |
| ATOM | 1138 | O | TRP | A | 165 | 45.827 | 61.956 | 6.148 | 1.00 | 10.10 | A |
| ATOM | 1139 | N | VAL | A | 166 | 44.761 | 60.896 | 4.470 | 1.00 | 9.73 | A |
| ATOM | 1140 | CA | VAL | A | 166 | 43.430 | 61.395 | 4.824 | 1.00 | 10.15 | A |
| ATOM | 1141 | CB | VAL | A | 166 | 43.021 | 62.605 | 3.929 | 1.00 | 9.63 | A |
| ATOM | 1142 | CG1 | VAL | A | 166 | 44.055 | 63.729 | 4.045 | 1.00 | 9.13 | A |
| ATOM | 1143 | CG2 | VAL | A | 166 | 42.896 | 62.159 | 2.463 | 1.00 | 9.97 | A |
| ATOM | 1144 | C | VAL | A | 166 | 42.400 | 60.287 | 4.594 | 1.00 | 10.04 | A |
| ATOM | 1145 | O | VAL | A | 166 | 42.758 | 59.151 | 4.295 | 1.00 | 10.65 | A |
| ATOM | 1146 | N | MET | A | 167 | 41.127 | 60.632 | 4.772 | 1.00 | 10.36 | A |
| ATOM | 1147 | CA | MET | A | 167 | 40.001 | 59.734 | 4.499 | 1.00 | 10.42 | A |
| ATOM | 1148 | CB | MET | A | 167 | 38.989 | 59.745 | 5.645 | 1.00 | 10.35 | A |
| ATOM | 1149 | CG | MET | A | 167 | 37.730 | 58.933 | 5.360 | 1.00 | 10.42 | A |
| ATOM | 1150 | SD | MET | A | 167 | 36.561 | 58.990 | 6.731 | 1.00 | 11.54 | A |
| ATOM | 1151 | CE | MET | A | 167 | 37.552 | 58.165 | 8.029 | 1.00 | 10.43 | A |
| ATOM | 1152 | C | MET | A | 167 | 39.454 | 60.498 | 3.298 | 1.00 | 10.44 | A |
| ATOM | 1153 | O | MET | A | 167 | 38.655 | 61.425 | 3.444 | 1.00 | 10.65 | A |
| ATOM | 1154 | N | PRO | A | 168 | 39.878 | 60.111 | 2.086 | 1.00 | 10.26 | A |
| ATOM | 1155 | CD | PRO | A | 168 | 40.630 | 58.883 | 1.758 | 1.00 | 9.75 | A |
| ATOM | 1156 | CA | PRO | A | 168 | 39.445 | 60.792 | 0.868 | 1.00 | 9.97 | A |
| ATOM | 1157 | CB | PRO | A | 168 | 40.371 | 60.202 | −0.189 | 1.00 | 9.78 | A |
| ATOM | 1158 | CG | PRO | A | 168 | 40.438 | 58.762 | 0.240 | 1.00 | 9.78 | A |
| ATOM | 1159 | C | PRO | A | 168 | 38.004 | 60.763 | 0.400 | 1.00 | 10.51 | A |
| ATOM | 1160 | O | PRO | A | 168 | 37.240 | 59.844 | 0.690 | 1.00 | 9.40 | A |
| ATOM | 1161 | N | ASP | A | 169 | 37.663 | 61.809 | −0.344 | 1.00 | 10.38 | A |
| ATOM | 1162 | CA | ASP | A | 169 | 36.370 | 61.915 | −0.981 | 1.00 | 10.54 | A |
| ATOM | 1163 | CB | ASP | A | 169 | 36.276 | 63.231 | −1.755 | 1.00 | 9.36 | A |
| ATOM | 1164 | CG | ASP | A | 169 | 35.158 | 63.227 | −2.776 | 1.00 | 9.43 | A |
| ATOM | 1165 | OD1 | ASP | A | 169 | 34.069 | 62.705 | −2.462 | 1.00 | 8.82 | A |
| ATOM | 1166 | OD2 | ASP | A | 169 | 35.363 | 63.758 | −3.885 | 1.00 | 9.69 | A |
| ATOM | 1167 | C | ASP | A | 169 | 36.463 | 60.746 | −1.959 | 1.00 | 11.05 | A |
| ATOM | 1168 | O | ASP | A | 169 | 37.565 | 60.380 | −2.373 | 1.00 | 10.71 | A |
| ATOM | 1169 | N | GLU | A | 170 | 35.332 | 60.151 | −2.322 | 1.00 | 11.08 | A |
| ATOM | 1170 | CA | GLU | A | 170 | 35.369 | 59.027 | −3.249 | 1.00 | 10.86 | A |
| ATOM | 1171 | CB | GLU | A | 170 | 34.767 | 57.779 | −2.578 | 1.00 | 11.08 | A |
| ATOM | 1172 | CG | GLU | A | 170 | 35.637 | 57.256 | −1.428 | 1.00 | 10.82 | A |
| ATOM | 1173 | CD | GLU | A | 170 | 35.030 | 56.067 | −0.694 | 1.00 | 10.92 | A |
| ATOM | 1174 | OE1 | GLU | A | 170 | 34.242 | 55.321 | −1.311 | 1.00 | 9.84 | A |
| ATOM | 1175 | OE2 | GLU | A | 170 | 35.368 | 55.869 | 0.497 | 1.00 | 10.95 | A |
| ATOM | 1176 | C | GLU | A | 170 | 34.668 | 59.325 | −4.569 | 1.00 | 10.67 | A |
| ATOM | 1177 | O | GLU | A | 170 | 34.655 | 58.491 | −5.473 | 1.00 | 10.61 | A |
| ATOM | 1178 | N | ALA | A | 171 | 34.116 | 60.529 | −4.690 | 1.00 | 10.81 | A |
| ATOM | 1179 | CA | ALA | A | 171 | 33.404 | 60.914 | −5.907 | 1.00 | 10.66 | A |
| ATOM | 1180 | CB | ALA | A | 171 | 32.167 | 61.722 | −5.542 | 1.00 | 11.39 | A |
| ATOM | 1181 | C | ALA | A | 171 | 34.239 | 61.697 | −6.920 | 1.00 | 10.93 | A |
| ATOM | 1182 | O | ALA | A | 171 | 34.284 | 61.351 | −8.102 | 1.00 | 11.16 | A |
| ATOM | 1183 | N | ASN | A | 172 | 34.893 | 62.753 | −6.447 | 1.00 | 10.71 | A |
| ATOM | 1184 | CA | ASN | A | 172 | 35.689 | 63.633 | −7.298 | 1.00 | 9.89 | A |
| ATOM | 1185 | CB | ASN | A | 172 | 35.645 | 65.048 | −6.723 | 1.00 | 9.48 | A |
| ATOM | 1186 | CG | ASN | A | 172 | 34.232 | 65.588 | −6.609 | 1.00 | 10.62 | A |
| ATOM | 1187 | OD1 | ASN | A | 172 | 33.575 | 65.848 | −7.616 | 1.00 | 10.69 | A |
| ATOM | 1188 | ND2 | ASN | A | 172 | 33.759 | 65.758 | −5.377 | 1.00 | 10.40 | A |
| ATOM | 1189 | C | ASN | A | 172 | 37.150 | 63.225 | −7.459 | 1.00 | 9.84 | A |
| ATOM | 1190 | O | ASN | A | 172 | 37.806 | 63.593 | −8.431 | 1.00 | 10.10 | A |
| ATOM | 1191 | N | SER | A | 173 | 37.657 | 62.470 | −6.498 | 1.00 | 9.67 | A |
| ATOM | 1192 | CA | SER | A | 173 | 39.055 | 62.050 | −6.511 | 1.00 | 9.66 | A |
| ATOM | 1193 | CB | SER | A | 173 | 39.378 | 61.359 | −5.194 | 1.00 | 8.38 | A |
| ATOM | 1194 | OG | SER | A | 173 | 38.460 | 60.305 | −4.959 | 1.00 | 9.36 | A |
| ATOM | 1195 | C | SER | A | 173 | 39.442 | 61.127 | −7.659 | 1.00 | 9.64 | A |
| ATOM | 1196 | O | SER | A | 173 | 38.672 | 60.246 | −8.050 | 1.00 | 8.79 | A |
| ATOM | 1197 | N | HIS | A | 174 | 40.639 | 61.338 | −8.203 | 1.00 | 9.71 | A |
| ATOM | 1198 | CA | HIS | A | 174 | 41.126 | 60.477 | −9.271 | 1.00 | 9.94 | A |
| ATOM | 1199 | CB | HIS | A | 174 | 42.138 | 61.215 | −10.139 | 1.00 | 10.93 | A |
| ATOM | 1200 | CG | HIS | A | 174 | 42.377 | 60.557 | −11.458 | 1.00 | 10.84 | A |
| ATOM | 1201 | CD2 | HIS | A | 174 | 41.963 | 60.888 | −12.704 | 1.00 | 10.41 | A |
| ATOM | 1202 | ND1 | HIS | A | 174 | 43.054 | 59.362 | −11.581 | 1.00 | 11.12 | A |
| ATOM | 1203 | CE1 | HIS | A | 174 | 43.043 | 58.984 | −12.846 | 1.00 | 11.26 | A |
| ATOM | 1204 | NE2 | HIS | A | 174 | 42.387 | 59.891 | −13.548 | 1.00 | 10.78 | A |
| ATOM | 1205 | C | HIS | A | 174 | 41.791 | 59.294 | −8.564 | 1.00 | 9.67 | A |
| ATOM | 1206 | O | HIS | A | 174 | 42.422 | 59.477 | −7.524 | 1.00 | 8.97 | A |
| ATOM | 1207 | N | TRP | A | 175 | 41.651 | 58.088 | −9.111 | 1.00 | 9.37 | A |
| ATOM | 1208 | CA | TRP | A | 175 | 42.233 | 56.920 | −8.458 | 1.00 | 9.44 | A |
| ATOM | 1209 | CB | TRP | A | 175 | 41.962 | 55.632 | −9.257 | 1.00 | 9.23 | A |
| ATOM | 1210 | CG | TRP | A | 175 | 42.792 | 55.451 | −10.506 | 1.00 | 9.39 | A |
| ATOM | 1211 | CD2 | TRP | A | 175 | 44.084 | 54.836 | −10.588 | 1.00 | 9.59 | A |
| ATOM | 1212 | CE2 | TRP | A | 175 | 44.480 | 54.871 | −11.945 | 1.00 | 9.59 | A |
| ATOM | 1213 | CE3 | TRP | A | 175 | 44.946 | 54.258 | −9.644 | 1.00 | 9.58 | A |
| ATOM | 1214 | CD1 | TRP | A | 175 | 42.465 | 55.828 | −11.777 | 1.00 | 9.27 | A |
| ATOM | 1215 | NE1 | TRP | A | 175 | 43.475 | 55.481 | −12.651 | 1.00 | 9.38 | A |
| ATOM | 1216 | CZ2 | TRP | A | 175 | 45.706 | 54.348 | −12.384 | 1.00 | 9.09 | A |
| ATOM | 1217 | CZ3 | TRP | A | 175 | 46.165 | 53.737 | −10.080 | 1.00 | 9.89 | A |
| ATOM | 1218 | CH2 | TRP | A | 175 | 46.531 | 53.788 | −11.442 | 1.00 | 10.05 | A |
| ATOM | 1219 | C | TRP | A | 175 | 43.725 | 57.083 | −8.223 | 1.00 | 9.46 | A |
| ATOM | 1220 | O | TRP | A | 175 | 44.261 | 56.583 | −7.233 | 1.00 | 9.08 | A |
| ATOM | 1221 | N | ARG | A | 176 | 44.401 | 57.788 | −9.124 | 1.00 | 9.19 | A |
| ATOM | 1222 | CA | ARG | A | 176 | 45.831 | 58.004 | −8.973 | 1.00 | 9.28 | A |
| ATOM | 1223 | CB | ARG | A | 176 | 46.373 | 58.761 | −10.191 | 1.00 | 9.85 | A |
| ATOM | 1224 | CG | ARG | A | 176 | 46.429 | 57.891 | −11.447 | 1.00 | 10.18 | A |
| ATOM | 1225 | CD | ARG | A | 176 | 46.402 | 58.710 | −12.722 | 1.00 | 10.64 | A |
| ATOM | 1226 | NE | ARG | A | 176 | 47.500 | 59.666 | −12.818 | 1.00 | 11.19 | A |
| ATOM | 1227 | CZ | ARG | A | 176 | 47.662 | 60.500 | −13.844 | 1.00 | 10.95 | A |
| ATOM | 1228 | NH1 | ARG | A | 176 | 46.796 | 60.486 | −14.853 | 1.00 | 11.00 | A |
| ATOM | 1229 | NH2 | ARG | A | 176 | 48.679 | 61.348 | −13.860 | 1.00 | 10.96 | A |
| ATOM | 1230 | C | ARG | A | 176 | 46.146 | 58.758 | −7.676 | 1.00 | 9.20 | A |
| ATOM | 1231 | O | ARG | A | 176 | 47.117 | 58.435 | −6.984 | 1.00 | 9.53 | A |
| ATOM | 1232 | N | ASN | A | 177 | 45.326 | 59.750 | −7.335 | 1.00 | 8.80 | A |
| ATOM | 1233 | CA | ASN | A | 177 | 45.561 | 60.511 | −6.108 | 1.00 | 8.75 | A |
| ATOM | 1234 | CB | ASN | A | 177 | 44.906 | 61.894 | −6.190 | 1.00 | 8.74 | A |
| ATOM | 1235 | CG | ASN | A | 177 | 45.577 | 62.784 | −7.217 | 1.00 | 10.06 | A |
| ATOM | 1236 | OD1 | ASN | A | 177 | 46.762 | 62.622 | −7.507 | 1.00 | 10.31 | A |
| ATOM | 1237 | ND2 | ASN | A | 177 | 44.826 | 63.734 | −7.766 | 1.00 | 11.06 | A |
| ATOM | 1238 | C | ASN | A | 177 | 45.077 | 59.759 | −4.870 | 1.00 | 8.84 | A |
| ATOM | 1239 | O | ASN | A | 177 | 45.578 | 59.981 | −3.763 | 1.00 | 8.42 | A |
| ATOM | 1240 | N | VAL | A | 178 | 44.100 | 58.876 | −5.049 | 1.00 | 8.65 | A |
| ATOM | 1241 | CA | VAL | A | 178 | 43.623 | 58.075 | −3.928 | 1.00 | 8.81 | A |
| ATOM | 1242 | CB | VAL | A | 178 | 42.408 | 57.198 | −4.329 | 1.00 | 9.88 | A |
| ATOM | 1243 | CG1 | VAL | A | 178 | 42.061 | 56.223 | −3.200 | 1.00 | 10.00 | A |
| ATOM | 1244 | CG2 | VAL | A | 178 | 41.211 | 58.086 | −4.645 | 1.00 | 9.98 | A |
| ATOM | 1245 | C | VAL | A | 178 | 44.804 | 57.173 | −3.550 | 1.00 | 8.79 | A |
| ATOM | 1246 | O | VAL | A | 178 | 45.104 | 56.982 | −2.371 | 1.00 | 9.06 | A |
| ATOM | 1247 | N | LEU | A | 179 | 45.481 | 56.633 | −4.564 | 1.00 | 8.48 | A |
| ATOM | 1248 | CA | LEU | A | 179 | 46.637 | 55.771 | −4.325 | 1.00 | 8.63 | A |
| ATOM | 1249 | CB | LEU | A | 179 | 47.104 | 55.103 | −5.624 | 1.00 | 9.00 | A |
| ATOM | 1250 | CG | LEU | A | 179 | 48.406 | 54.287 | −5.506 | 1.00 | 9.81 | A |
| ATOM | 1251 | CD1 | LEU | A | 179 | 48.243 | 53.164 | −4.479 | 1.00 | 9.86 | A |
| ATOM | 1252 | CD2 | LEU | A | 179 | 48.764 | 53.714 | −6.872 | 1.00 | 10.42 | A |
| ATOM | 1253 | C | LEU | A | 179 | 47.790 | 56.575 | −3.739 | 1.00 | 8.69 | A |
| ATOM | 1254 | O | LEU | A | 179 | 48.494 | 56.104 | −2.839 | 1.00 | 9.30 | A |
| ATOM | 1255 | N | LEU | A | 180 | 47.983 | 57.789 | −4.250 | 1.00 | 8.07 | A |
| ATOM | 1256 | CA | LEU | A | 180 | 49.063 | 58.643 | −3.765 | 1.00 | 8.58 | A |
| ATOM | 1257 | CB | LEU | A | 180 | 49.064 | 59.980 | −4.521 | 1.00 | 8.65 | A |
| ATOM | 1258 | CG | LEU | A | 180 | 50.203 | 60.946 | −4.175 | 1.00 | 9.33 | A |
| ATOM | 1259 | CD1 | LEU | A | 180 | 51.511 | 60.398 | −4.746 | 1.00 | 9.58 | A |
| ATOM | 1260 | CD2 | LEU | A | 180 | 49.913 | 62.335 | −4.735 | 1.00 | 9.53 | A |
| ATOM | 1261 | C | LEU | A | 180 | 48.926 | 58.903 | −2.262 | 1.00 | 8.44 | A |
| ATOM | 1262 | O | LEU | A | 180 | 49.881 | 58.708 | −1.501 | 1.00 | 9.34 | A |
| ATOM | 1263 | N | GLN | A | 181 | 47.743 | 59.329 | −1.825 | 1.00 | 7.79 | A |
| ATOM | 1264 | CA | GLN | A | 181 | 47.550 | 59.623 | −0.408 | 1.00 | 8.83 | A |
| ATOM | 1265 | CB | GLN | A | 181 | 46.254 | 60.426 | −0.184 | 1.00 | 8.07 | A |
| ATOM | 1266 | CG | GLN | A | 181 | 44.935 | 59.689 | −0.427 | 1.00 | 9.20 | A |
| ATOM | 1267 | CD | GLN | A | 181 | 44.568 | 58.748 | 0.710 | 1.00 | 9.64 | A |
| ATOM | 1268 | OE1 | GLN | A | 181 | 44.834 | 59.035 | 1.884 | 1.00 | 10.23 | A |
| ATOM | 1269 | NE2 | GLN | A | 181 | 43.940 | 57.627 | 0.371 | 1.00 | 8.92 | A |
| ATOM | 1270 | C | GLN | A | 181 | 47.591 | 58.379 | 0.474 | 1.00 | 8.83 | A |
| ATOM | 1271 | O | GLN | A | 181 | 48.063 | 58.448 | 1.607 | 1.00 | 9.20 | A |
| ATOM | 1272 | N | LEU | A | 182 | 47.110 | 57.246 | −0.035 | 1.00 | 8.67 | A |
| ATOM | 1273 | CA | LEU | A | 182 | 47.155 | 56.012 | 0.743 | 1.00 | 8.95 | A |
| ATOM | 1274 | CB | LEU | A | 182 | 46.432 | 54.871 | 0.011 | 1.00 | 8.61 | A |
| ATOM | 1275 | CG | LEU | A | 182 | 46.498 | 53.481 | 0.664 | 1.00 | 8.86 | A |
| ATOM | 1276 | CD1 | LEU | A | 182 | 45.753 | 53.482 | 1.997 | 1.00 | 8.49 | A |
| ATOM | 1277 | CD2 | LEU | A | 182 | 45.889 | 52.443 | −0.283 | 1.00 | 9.99 | A |
| ATOM | 1278 | C | LEU | A | 182 | 48.626 | 55.642 | 0.933 | 1.00 | 9.47 | A |
| ATOM | 1279 | O | LEU | A | 182 | 49.058 | 55.298 | 2.033 | 1.00 | 9.33 | A |
| ATOM | 1280 | N | THR | A | 183 | 49.395 | 55.737 | −0.149 | 1.00 | 9.38 | A |
| ATOM | 1281 | CA | THR | A | 183 | 50.815 | 55.407 | −0.113 | 1.00 | 10.00 | A |
| ATOM | 1282 | CB | THR | A | 183 | 51.440 | 55.484 | −1.537 | 1.00 | 10.83 | A |
| ATOM | 1283 | OG1 | THR | A | 183 | 50.713 | 54.627 | −2.430 | 1.00 | 9.77 | A |
| ATOM | 1284 | CG2 | THR | A | 183 | 52.906 | 55.045 | −1.506 | 1.00 | 11.31 | A |
| ATOM | 1285 | C | THR | A | 183 | 51.570 | 56.352 | 0.825 | 1.00 | 10.11 | A |
| ATOM | 1286 | O | THR | A | 183 | 52.438 | 55.923 | 1.595 | 1.00 | 9.89 | A |
| ATOM | 1287 | N | GLU | A | 184 | 51.239 | 57.638 | 0.772 | 1.00 | 10.04 | A |
| ATOM | 1288 | CA | GLU | A | 184 | 51.914 | 58.613 | 1.624 | 1.00 | 10.88 | A |
| ATOM | 1289 | CB | GLU | A | 184 | 51.370 | 60.019 | 1.345 | 1.00 | 11.50 | A |
| ATOM | 1290 | CG | GLU | A | 184 | 52.226 | 61.167 | 1.881 | 1.00 | 12.25 | A |
| ATOM | 1291 | CD | GLU | A | 184 | 53.620 | 61.223 | 1.262 | 1.00 | 13.15 | A |
| ATOM | 1292 | OE1 | GLU | A | 184 | 53.768 | 60.907 | 0.063 | 1.00 | 12.76 | A |
| ATOM | 1293 | OE2 | GLU | A | 184 | 54.568 | 61.607 | 1.977 | 1.00 | 13.95 | A |
| ATOM | 1294 | C | GLU | A | 184 | 51.714 | 58.248 | 3.099 | 1.00 | 10.59 | A |
| ATOM | 1295 | O | GLU | A | 184 | 52.664 | 58.249 | 3.885 | 1.00 | 10.65 | A |
| ATOM | 1296 | N | GLY | A | 185 | 50.479 | 57.922 | 3.463 | 1.00 | 10.39 | A |
| ATOM | 1297 | CA | GLY | A | 185 | 50.182 | 57.557 | 4.839 | 1.00 | 10.33 | A |
| ATOM | 1298 | C | GLY | A | 185 | 50.768 | 56.222 | 5.268 | 1.00 | 10.94 | A |
| ATOM | 1299 | O | GLY | A | 185 | 51.355 | 56.110 | 6.351 | 1.00 | 9.76 | A |
| ATOM | 1300 | N | GLN | A | 186 | 50.622 | 55.200 | 4.430 | 1.00 | 10.45 | A |
| ATOM | 1301 | CA | GLN | A | 186 | 51.144 | 53.884 | 4.788 | 1.00 | 11.43 | A |
| ATOM | 1302 | CB | GLN | A | 186 | 50.560 | 52.803 | 3.874 | 1.00 | 11.23 | A |
| ATOM | 1303 | CG | GLN | A | 186 | 49.047 | 52.623 | 4.010 | 1.00 | 12.83 | A |
| ATOM | 1304 | CD | GLN | A | 186 | 48.618 | 51.182 | 3.793 | 1.00 | 13.65 | A |
| ATOM | 1305 | OE1 | GLN | A | 186 | 49.190 | 50.479 | 2.965 | 1.00 | 15.97 | A |
| ATOM | 1306 | NE2 | GLN | A | 186 | 47.602 | 50.741 | 4.528 | 1.00 | 13.28 | A |
| ATOM | 1307 | C | GLN | A | 186 | 52.668 | 53.800 | 4.781 | 1.00 | 11.78 | A |
| ATOM | 1308 | O | GLN | A | 186 | 53.255 | 53.037 | 5.548 | 1.00 | 11.71 | A |
| ATOM | 1309 | N | THR | A | 187 | 53.314 | 54.571 | 3.916 | 1.00 | 11.96 | A |
| ATOM | 1310 | CA | THR | A | 187 | 54.770 | 54.548 | 3.875 | 1.00 | 11.50 | A |
| ATOM | 1311 | CB | THR | A | 187 | 55.300 | 55.350 | 2.672 | 1.00 | 11.86 | A |
| ATOM | 1312 | OG1 | THR | A | 187 | 54.843 | 54.733 | 1.460 | 1.00 | 10.52 | A |
| ATOM | 1313 | CG2 | THR | A | 187 | 56.829 | 55.372 | 2.666 | 1.00 | 10.99 | A |
| ATOM | 1314 | C | THR | A | 187 | 55.289 | 55.130 | 5.191 | 1.00 | 12.09 | A |
| ATOM | 1315 | O | THR | A | 187 | 56.252 | 54.627 | 5.770 | 1.00 | 11.42 | A |
| ATOM | 1316 | N | TRP | A | 188 | 54.632 | 56.180 | 5.674 | 1.00 | 12.12 | A |
| ATOM | 1317 | CA | TRP | A | 188 | 55.033 | 56.792 | 6.936 | 1.00 | 12.70 | A |
| ATOM | 1318 | CB | TRP | A | 188 | 54.184 | 58.034 | 7.236 | 1.00 | 13.41 | A |
| ATOM | 1319 | CG | TRP | A | 188 | 54.647 | 58.790 | 8.456 | 1.00 | 14.39 | A |
| ATOM | 1320 | CD2 | TRP | A | 188 | 54.293 | 58.517 | 9.818 | 1.00 | 14.53 | A |
| ATOM | 1321 | CE2 | TRP | A | 188 | 55.038 | 59.403 | 10.630 | 1.00 | 15.63 | A |
| ATOM | 1322 | CE3 | TRP | A | 188 | 53.423 | 57.606 | 10.431 | 1.00 | 14.76 | A |
| ATOM | 1323 | CD1 | TRP | A | 188 | 55.562 | 59.807 | 8.495 | 1.00 | 14.92 | A |
| ATOM | 1324 | NE1 | TRP | A | 188 | 55.804 | 60.178 | 9.798 | 1.00 | 14.82 | A |
| ATOM | 1325 | CZ2 | TRP | A | 188 | 54.940 | 59.402 | 12.025 | 1.00 | 14.74 | A |
| ATOM | 1326 | CZ3 | TRP | A | 188 | 53.327 | 57.604 | 11.824 | 1.00 | 15.69 | A |
| ATOM | 1327 | CH2 | TRP | A | 188 | 54.081 | 58.497 | 12.602 | 1.00 | 15.84 | A |
| ATOM | 1328 | C | TRP | A | 188 | 54.837 | 55.763 | 8.052 | 1.00 | 12.21 | A |
| ATOM | 1329 | O | TRP | A | 188 | 55.725 | 55.555 | 8.875 | 1.00 | 12.03 | A |
| ATOM | 1330 | N | LEU | A | 189 | 53.672 | 55.116 | 8.071 | 1.00 | 12.09 | A |
| ATOM | 1331 | CA | LEU | A | 189 | 53.375 | 54.112 | 9.091 | 1.00 | 12.32 | A |
| ATOM | 1332 | CB | LEU | A | 189 | 51.966 | 53.538 | 8.896 | 1.00 | 11.84 | A |
| ATOM | 1333 | CG | LEU | A | 189 | 50.798 | 54.434 | 9.314 | 1.00 | 11.10 | A |
| ATOM | 1334 | CD1 | LEU | A | 189 | 49.475 | 53.718 | 9.013 | 1.00 | 10.75 | A |
| ATOM | 1335 | CD2 | LEU | A | 189 | 50.912 | 54.757 | 10.806 | 1.00 | 10.87 | A |
| ATOM | 1336 | C | LEU | A | 189 | 54.376 | 52.962 | 9.111 | 1.00 | 12.67 | A |
| ATOM | 1337 | O | LEU | A | 189 | 54.792 | 52.513 | 10.181 | 1.00 | 12.84 | A |
| ATOM | 1338 | N | LYS | A | 190 | 54.757 | 52.473 | 7.936 | 1.00 | 12.75 | A |
| ATOM | 1339 | CA | LYS | A | 190 | 55.709 | 51.370 | 7.882 | 1.00 | 15.02 | A |
| ATOM | 1340 | CB | LYS | A | 190 | 55.916 | 50.886 | 6.444 | 1.00 | 16.33 | A |
| ATOM | 1341 | CG | LYS | A | 190 | 56.750 | 49.605 | 6.350 | 1.00 | 18.79 | A |
| ATOM | 1342 | CD | LYS | A | 190 | 56.958 | 49.174 | 4.906 | 1.00 | 21.47 | A |
| ATOM | 1343 | CE | LYS | A | 190 | 57.529 | 47.761 | 4.821 | 1.00 | 24.11 | A |
| ATOM | 1344 | NZ | LYS | A | 190 | 58.713 | 47.598 | 5.708 | 1.00 | 26.25 | A |
| ATOM | 1345 | C | LYS | A | 190 | 57.052 | 51.793 | 8.464 | 1.00 | 15.60 | A |
| ATOM | 1346 | O | LYS | A | 190 | 57.654 | 51.067 | 9.254 | 1.00 | 15.07 | A |
| ATOM | 1347 | N | GLN | A | 191 | 57.514 | 52.974 | 8.075 | 1.00 | 16.02 | A |
| ATOM | 1348 | CA | GLN | A | 191 | 58.794 | 53.477 | 8.548 | 1.00 | 17.78 | A |
| ATOM | 1349 | CB | GLN | A | 191 | 59.199 | 54.722 | 7.750 | 1.00 | 19.02 | A |
| ATOM | 1350 | CG | GLN | A | 191 | 60.526 | 55.324 | 8.202 | 1.00 | 23.22 | A |
| ATOM | 1351 | CD | GLN | A | 191 | 60.944 | 56.540 | 7.390 | 1.00 | 24.68 | A |
| ATOM | 1352 | OE1 | GLN | A | 191 | 61.989 | 57.140 | 7.651 | 1.00 | 27.33 | A |
| ATOM | 1353 | NE2 | GLN | A | 191 | 60.132 | 56.909 | 6.402 | 1.00 | 25.58 | A |
| ATOM | 1354 | C | GLN | A | 191 | 58.848 | 53.800 | 10.041 | 1.00 | 17.91 | A |
| ATOM | 1355 | O | GLN | A | 191 | 59.810 | 53.434 | 10.717 | 1.00 | 18.33 | A |
| ATOM | 1356 | N | PHE | A | 192 | 57.827 | 54.474 | 10.563 | 1.00 | 17.35 | A |
| ATOM | 1357 | CA | PHE | A | 192 | 57.841 | 54.854 | 11.974 | 1.00 | 17.36 | A |
| ATOM | 1358 | CB | PHE | A | 192 | 57.419 | 56.320 | 12.116 | 1.00 | 16.34 | A |
| ATOM | 1359 | CG | PHE | A | 192 | 58.324 | 57.279 | 11.402 | 1.00 | 16.29 | A |
| ATOM | 1360 | CD1 | PHE | A | 192 | 58.020 | 57.717 | 10.115 | 1.00 | 15.35 | A |
| ATOM | 1361 | CD2 | PHE | A | 192 | 59.495 | 57.726 | 12.004 | 1.00 | 16.10 | A |
| ATOM | 1362 | CE1 | PHE | A | 192 | 58.867 | 58.585 | 9.439 | 1.00 | 16.22 | A |
| ATOM | 1363 | CE2 | PHE | A | 192 | 60.354 | 58.598 | 11.334 | 1.00 | 17.05 | A |
| ATOM | 1364 | CZ | PHE | A | 192 | 60.040 | 59.029 | 10.050 | 1.00 | 16.60 | A |
| ATOM | 1365 | C | PHE | A | 192 | 57.045 | 54.009 | 12.972 | 1.00 | 18.24 | A |
| ATOM | 1366 | O | PHE | A | 192 | 57.395 | 53.972 | 14.154 | 1.00 | 18.88 | A |
| ATOM | 1367 | N | MET | A | 193 | 55.989 | 53.340 | 12.519 | 1.00 | 17.95 | A |
| ATOM | 1368 | CA | MET | A | 193 | 55.170 | 52.518 | 13.418 | 1.00 | 19.29 | A |
| ATOM | 1369 | CB | MET | A | 193 | 53.684 | 52.877 | 13.282 | 1.00 | 20.31 | A |
| ATOM | 1370 | CG | MET | A | 193 | 53.272 | 54.222 | 13.862 | 1.00 | 21.04 | A |
| ATOM | 1371 | SD | MET | A | 193 | 53.652 | 54.404 | 15.629 | 1.00 | 25.98 | A |
| ATOM | 1372 | CE | MET | A | 193 | 54.978 | 55.547 | 15.464 | 1.00 | 22.14 | A |
| ATOM | 1373 | C | MET | A | 193 | 55.336 | 51.022 | 13.153 | 1.00 | 19.68 | A |
| ATOM | 1374 | O | MET | A | 193 | 54.858 | 50.189 | 13.928 | 1.00 | 18.38 | A |
| ATOM | 1375 | N | ASN | A | 194 | 56.001 | 50.693 | 12.050 | 1.00 | 20.20 | A |
| ATOM | 1376 | CA | ASN | A | 194 | 56.234 | 49.306 | 11.662 | 1.00 | 22.12 | A |
| ATOM | 1377 | CB | ASN | A | 194 | 57.165 | 48.627 | 12.676 | 1.00 | 26.86 | A |
| ATOM | 1378 | CG | ASN | A | 194 | 57.617 | 47.247 | 12.225 | 1.00 | 32.79 | A |
| ATOM | 1379 | OD1 | ASN | A | 194 | 57.780 | 46.999 | 11.028 | 1.00 | 31.85 | A |
| ATOM | 1380 | ND2 | ASN | A | 194 | 57.832 | 46.357 | 13.191 | 1.00 | 38.94 | A |
| ATOM | 1381 | C | ASN | A | 194 | 54.929 | 48.517 | 11.534 | 1.00 | 20.58 | A |
| ATOM | 1382 | O | ASN | A | 194 | 54.833 | 47.373 | 11.978 | 1.00 | 19.15 | A |
| ATOM | 1383 | N | VAL | A | 195 | 53.920 | 49.139 | 10.935 | 1.00 | 18.58 | A |
| ATOM | 1384 | CA | VAL | A | 195 | 52.634 | 48.479 | 10.742 | 1.00 | 17.61 | A |
| ATOM | 1385 | CB | VAL | A | 195 | 51.628 | 48.794 | 11.888 | 1.00 | 17.65 | A |
| ATOM | 1386 | CG1 | VAL | A | 195 | 52.173 | 48.301 | 13.227 | 1.00 | 19.15 | A |
| ATOM | 1387 | CG2 | VAL | A | 195 | 51.342 | 50.288 | 11.941 | 1.00 | 18.14 | A |
| ATOM | 1388 | C | VAL | A | 195 | 52.006 | 48.928 | 9.428 | 1.00 | 16.13 | A |
| ATOM | 1389 | O | VAL | A | 195 | 52.232 | 50.051 | 8.975 | 1.00 | 15.25 | A |
| ATOM | 1390 | N | THR | A | 196 | 51.229 | 48.034 | 8.825 | 1.00 | 14.72 | A |
| ATOM | 1391 | CA | THR | A | 196 | 50.527 | 48.309 | 7.574 | 1.00 | 14.36 | A |
| ATOM | 1392 | CB | THR | A | 196 | 51.159 | 47.554 | 6.377 | 1.00 | 14.11 | A |
| ATOM | 1393 | OG1 | THR | A | 196 | 52.516 | 47.978 | 6.196 | 1.00 | 14.55 | A |
| ATOM | 1394 | CG2 | THR | A | 196 | 50.374 | 47.830 | 5.105 | 1.00 | 15.25 | A |
| ATOM | 1395 | C | THR | A | 196 | 49.093 | 47.818 | 7.746 | 1.00 | 13.73 | A |
| ATOM | 1396 | O | THR | A | 196 | 48.845 | 46.613 | 7.787 | 1.00 | 13.60 | A |
| ATOM | 1397 | N | PRO | A | 197 | 48.130 | 48.745 | 7.859 | 1.00 | 13.55 | A |
| ATOM | 1398 | CD | PRO | A | 197 | 48.302 | 50.207 | 7.944 | 1.00 | 13.01 | A |
| ATOM | 1399 | CA | PRO | A | 197 | 46.722 | 48.375 | 8.029 | 1.00 | 13.52 | A |
| ATOM | 1400 | CB | PRO | A | 197 | 46.014 | 49.726 | 8.085 | 1.00 | 13.17 | A |
| ATOM | 1401 | CG | PRO | A | 197 | 47.057 | 50.632 | 8.681 | 1.00 | 12.88 | A |
| ATOM | 1402 | C | PRO | A | 197 | 46.181 | 47.507 | 6.898 | 1.00 | 13.94 | A |
| ATOM | 1403 | O | PRO | A | 197 | 46.536 | 47.699 | 5.733 | 1.00 | 13.93 | A |
| ATOM | 1404 | N | THR | A | 198 | 45.335 | 46.542 | 7.249 | 1.00 | 13.37 | A |
| ATOM | 1405 | CA | THR | A | 198 | 44.721 | 45.677 | 6.250 | 1.00 | 13.41 | A |
| ATOM | 1406 | CB | THR | A | 198 | 45.065 | 44.185 | 6.457 | 1.00 | 13.58 | A |
| ATOM | 1407 | OG1 | THR | A | 198 | 44.601 | 43.759 | 7.740 | 1.00 | 13.61 | A |
| ATOM | 1408 | CG2 | THR | A | 198 | 46.567 | 43.963 | 6.342 | 1.00 | 14.13 | A |
| ATOM | 1409 | C | THR | A | 198 | 43.210 | 45.841 | 6.322 | 1.00 | 11.99 | A |
| ATOM | 1410 | O | THR | A | 198 | 42.473 | 45.152 | 5.623 | 1.00 | 11.91 | A |
| ATOM | 1411 | N | ALA | A | 199 | 42.760 | 46.757 | 7.179 | 1.00 | 11.92 | A |
| ATOM | 1412 | CA | ALA | A | 199 | 41.336 | 47.050 | 7.332 | 1.00 | 11.29 | A |
| ATOM | 1413 | CB | ALA | A | 199 | 40.856 | 46.659 | 8.736 | 1.00 | 11.40 | A |
| ATOM | 1414 | C | ALA | A | 199 | 41.110 | 48.547 | 7.092 | 1.00 | 11.77 | A |
| ATOM | 1415 | O | ALA | A | 199 | 41.807 | 49.386 | 7.667 | 1.00 | 12.44 | A |
| ATOM | 1416 | N | SER | A | 200 | 40.135 | 48.879 | 6.252 | 1.00 | 10.98 | A |
| ATOM | 1417 | CA | SER | A | 200 | 39.844 | 50.276 | 5.937 | 1.00 | 11.61 | A |
| ATOM | 1418 | CB | SER | A | 200 | 39.680 | 50.459 | 4.426 | 1.00 | 11.69 | A |
| ATOM | 1419 | OG | SER | A | 200 | 39.531 | 51.830 | 4.097 | 1.00 | 12.70 | A |
| ATOM | 1420 | C | SER | A | 200 | 38.607 | 50.795 | 6.658 | 1.00 | 11.42 | A |
| ATOM | 1421 | O | SER | A | 200 | 37.635 | 50.057 | 6.871 | 1.00 | 11.38 | A |
| ATOM | 1422 | N | TRP | A | 201 | 38.654 | 52.077 | 7.008 | 1.00 | 10.70 | A |
| ATOM | 1423 | CA | TRP | A | 201 | 37.592 | 52.759 | 7.745 | 1.00 | 11.31 | A |
| ATOM | 1424 | CB | TRP | A | 201 | 38.110 | 52.997 | 9.176 | 1.00 | 11.10 | A |
| ATOM | 1425 | CG | TRP | A | 201 | 37.296 | 53.856 | 10.113 | 1.00 | 12.37 | A |
| ATOM | 1426 | CD2 | TRP | A | 201 | 36.309 | 53.406 | 11.051 | 1.00 | 12.43 | A |
| ATOM | 1427 | CE2 | TRP | A | 201 | 35.917 | 54.529 | 11.819 | 1.00 | 13.19 | A |
| ATOM | 1428 | CE3 | TRP | A | 201 | 35.722 | 52.162 | 11.322 | 1.00 | 13.10 | A |
| ATOM | 1429 | CD1 | TRP | A | 201 | 37.448 | 55.199 | 10.338 | 1.00 | 12.79 | A |
| ATOM | 1430 | NE1 | TRP | A | 201 | 36.627 | 55.608 | 11.361 | 1.00 | 12.52 | A |
| ATOM | 1431 | CZ2 | TRP | A | 201 | 34.964 | 54.444 | 12.841 | 1.00 | 12.77 | A |
| ATOM | 1432 | CZ3 | TRP | A | 201 | 34.772 | 52.077 | 12.344 | 1.00 | 13.70 | A |
| ATOM | 1433 | CH2 | TRP | A | 201 | 34.405 | 53.215 | 13.089 | 1.00 | 13.30 | A |
| ATOM | 1434 | C | TRP | A | 201 | 37.225 | 54.074 | 7.040 | 1.00 | 11.37 | A |
| ATOM | 1435 | O | TRP | A | 201 | 37.995 | 55.031 | 7.058 | 1.00 | 12.26 | A |
| ATOM | 1436 | N | ALA | A | 202 | 36.053 | 54.102 | 6.408 | 1.00 | 11.54 | A |
| ATOM | 1437 | CA | ALA | A | 202 | 35.578 | 55.285 | 5.686 | 1.00 | 11.82 | A |
| ATOM | 1438 | CB | ALA | A | 202 | 35.620 | 55.026 | 4.180 | 1.00 | 12.16 | A |
| ATOM | 1439 | C | ALA | A | 202 | 34.152 | 55.613 | 6.129 | 1.00 | 11.83 | A |
| ATOM | 1440 | O | ALA | A | 202 | 33.184 | 55.165 | 5.519 | 1.00 | 11.26 | A |
| ATOM | 1441 | N | ILE | A | 203 | 34.039 | 56.420 | 7.181 | 1.00 | 11.80 | A |
| ATOM | 1442 | CA | ILE | A | 203 | 32.747 | 56.774 | 7.762 | 1.00 | 11.97 | A |
| ATOM | 1443 | CB | ILE | A | 203 | 32.830 | 56.770 | 9.311 | 1.00 | 12.02 | A |
| ATOM | 1444 | CG2 | ILE | A | 203 | 33.134 | 55.350 | 9.821 | 1.00 | 12.31 | A |
| ATOM | 1445 | CG1 | ILE | A | 203 | 33.914 | 57.756 | 9.772 | 1.00 | 12.87 | A |
| ATOM | 1446 | CD1 | ILE | A | 203 | 33.998 | 57.941 | 11.286 | 1.00 | 13.55 | A |
| ATOM | 1447 | C | ILE | A | 203 | 32.115 | 58.101 | 7.347 | 1.00 | 11.95 | A |
| ATOM | 1448 | O | ILE | A | 203 | 30.937 | 58.324 | 7.633 | 1.00 | 11.82 | A |
| ATOM | 1449 | N | ASP | A | 204 | 32.861 | 58.978 | 6.677 | 1.00 | 11.66 | A |
| ATOM | 1450 | CA | ASP | A | 204 | 32.278 | 60.269 | 6.305 | 1.00 | 12.21 | A |
| ATOM | 1451 | CB | ASP | A | 204 | 32.986 | 61.406 | 7.053 | 1.00 | 11.72 | A |
| ATOM | 1452 | CG | ASP | A | 204 | 32.058 | 62.589 | 7.336 | 1.00 | 11.97 | A |
| ATOM | 1453 | OD1 | ASP | A | 204 | 32.557 | 63.712 | 7.559 | 1.00 | 12.03 | A |
| ATOM | 1454 | OD2 | ASP | A | 204 | 30.823 | 62.401 | 7.351 | 1.00 | 11.68 | A |
| ATOM | 1455 | C | ASP | A | 204 | 32.162 | 60.651 | 4.824 | 1.00 | 12.19 | A |
| ATOM | 1456 | O | ASP | A | 204 | 31.420 | 61.580 | 4.496 | 1.00 | 12.41 | A |
| ATOM | 1457 | N | PRO | A | 205 | 32.888 | 59.969 | 3.911 | 1.00 | 12.60 | A |
| ATOM | 1458 | CD | PRO | A | 205 | 33.888 | 58.895 | 4.047 | 1.00 | 12.94 | A |
| ATOM | 1459 | CA | PRO | A | 205 | 32.739 | 60.366 | 2.500 | 1.00 | 12.61 | A |
| ATOM | 1460 | CB | PRO | A | 205 | 33.573 | 59.323 | 1.758 | 1.00 | 12.97 | A |
| ATOM | 1461 | CG | PRO | A | 205 | 34.666 | 59.017 | 2.747 | 1.00 | 13.56 | A |
| ATOM | 1462 | C | PRO | A | 205 | 31.257 | 60.328 | 2.109 | 1.00 | 12.65 | A |
| ATOM | 1463 | O | PRO | A | 205 | 30.520 | 59.451 | 2.557 | 1.00 | 13.12 | A |
| ATOM | 1464 | N | PHE | A | 206 | 30.828 | 61.266 | 1.267 | 1.00 | 12.48 | A |
| ATOM | 1465 | CA | PHE | A | 206 | 29.418 | 61.367 | 0.872 | 1.00 | 11.65 | A |
| ATOM | 1466 | CB | PHE | A | 206 | 29.094 | 62.829 | 0.542 | 1.00 | 10.97 | A |
| ATOM | 1467 | CG | PHE | A | 206 | 29.933 | 63.823 | 1.310 | 1.00 | 11.31 | A |
| ATOM | 1468 | CD1 | PHE | A | 206 | 30.170 | 63.652 | 2.673 | 1.00 | 10.59 | A |
| ATOM | 1469 | CD2 | PHE | A | 206 | 30.497 | 64.926 | 0.668 | 1.00 | 11.93 | A |
| ATOM | 1470 | CE1 | PHE | A | 206 | 30.957 | 64.559 | 3.385 | 1.00 | 10.69 | A |
| ATOM | 1471 | CE2 | PHE | A | 206 | 31.286 | 65.842 | 1.372 | 1.00 | 11.78 | A |
| ATOM | 1472 | CZ | PHE | A | 206 | 31.518 | 65.656 | 2.734 | 1.00 | 10.49 | A |
| ATOM | 1473 | C | PHE | A | 206 | 29.064 | 60.456 | −0.306 | 1.00 | 11.76 | A |
| ATOM | 1474 | O | PHE | A | 206 | 28.869 | 60.914 | −1.431 | 1.00 | 11.63 | A |
| ATOM | 1475 | N | GLY | A | 207 | 28.946 | 59.164 | −0.017 | 1.00 | 11.94 | A |
| ATOM | 1476 | CA | GLY | A | 207 | 28.677 | 58.182 | −1.053 | 1.00 | 11.86 | A |
| ATOM | 1477 | C | GLY | A | 207 | 29.978 | 57.409 | −1.186 | 1.00 | 11.58 | A |
| ATOM | 1478 | O | GLY | A | 207 | 31.034 | 57.942 | −0.840 | 1.00 | 11.47 | A |
| ATOM | 1479 | N | HIS | A | 208 | 29.922 | 56.173 | −1.681 | 1.00 | 11.57 | A |
| ATOM | 1480 | CA | HIS | A | 208 | 31.125 | 55.351 | −1.800 | 1.00 | 11.22 | A |
| ATOM | 1481 | CB | HIS | A | 208 | 31.074 | 54.238 | −0.753 | 1.00 | 11.82 | A |
| ATOM | 1482 | CG | HIS | A | 208 | 31.157 | 54.741 | 0.654 | 1.00 | 12.72 | A |
| ATOM | 1483 | CD2 | HIS | A | 208 | 30.195 | 54.971 | 1.578 | 1.00 | 12.86 | A |
| ATOM | 1484 | ND1 | HIS | A | 208 | 32.349 | 55.099 | 1.245 | 1.00 | 13.33 | A |
| ATOM | 1485 | CE1 | HIS | A | 208 | 32.118 | 55.527 | 2.473 | 1.00 | 13.14 | A |
| ATOM | 1486 | NE2 | HIS | A | 208 | 30.819 | 55.460 | 2.700 | 1.00 | 12.64 | A |
| ATOM | 1487 | C | HIS | A | 208 | 31.364 | 54.757 | −3.181 | 1.00 | 10.90 | A |
| ATOM | 1488 | O | HIS | A | 208 | 30.421 | 54.409 | −3.899 | 1.00 | 11.18 | A |
| ATOM | 1489 | N | SER | A | 209 | 32.638 | 54.629 | −3.536 | 1.00 | 10.51 | A |
| ATOM | 1490 | CA | SER | A | 209 | 33.037 | 54.107 | −4.841 | 1.00 | 10.62 | A |
| ATOM | 1491 | CB | SER | A | 209 | 33.915 | 55.137 | −5.554 | 1.00 | 10.69 | A |
| ATOM | 1492 | OG | SER | A | 209 | 34.484 | 54.588 | −6.735 | 1.00 | 11.16 | A |
| ATOM | 1493 | C | SER | A | 209 | 33.795 | 52.783 | −4.783 | 1.00 | 10.51 | A |
| ATOM | 1494 | O | SER | A | 209 | 34.600 | 52.561 | −3.875 | 1.00 | 9.87 | A |
| ATOM | 1495 | N | PRO | A | 210 | 33.550 | 51.890 | −5.762 | 1.00 | 10.42 | A |
| ATOM | 1496 | CD | PRO | A | 210 | 32.556 | 52.009 | −6.845 | 1.00 | 10.36 | A |
| ATOM | 1497 | CA | PRO | A | 210 | 34.222 | 50.589 | −5.820 | 1.00 | 10.49 | A |
| ATOM | 1498 | CB | PRO | A | 210 | 33.452 | 49.846 | −6.910 | 1.00 | 10.13 | A |
| ATOM | 1499 | CG | PRO | A | 210 | 33.024 | 50.951 | −7.828 | 1.00 | 10.41 | A |
| ATOM | 1500 | C | PRO | A | 210 | 35.705 | 50.759 | −6.147 | 1.00 | 10.64 | A |
| ATOM | 1501 | O | PRO | A | 210 | 36.481 | 49.808 | −6.066 | 1.00 | 10.74 | A |
| ATOM | 1502 | N | THR | A | 211 | 36.103 | 51.972 | −6.523 | 1.00 | 9.89 | A |
| ATOM | 1503 | CA | THR | A | 211 | 37.514 | 52.211 | −6.792 | 1.00 | 10.11 | A |
| ATOM | 1504 | CB | THR | A | 211 | 37.772 | 53.661 | −7.261 | 1.00 | 9.96 | A |
| ATOM | 1505 | OG1 | THR | A | 211 | 37.237 | 53.833 | −8.578 | 1.00 | 9.80 | A |
| ATOM | 1506 | CG2 | THR | A | 211 | 39.270 | 53.961 | −7.287 | 1.00 | 9.72 | A |
| ATOM | 1507 | C | THR | A | 211 | 38.309 | 51.956 | −5.504 | 1.00 | 9.81 | A |
| ATOM | 1508 | O | THR | A | 211 | 39.479 | 51.571 | −5.552 | 1.00 | 9.54 | A |
| ATOM | 1509 | N | MET | A | 212 | 37.672 | 52.156 | −4.353 | 1.00 | 10.26 | A |
| ATOM | 1510 | CA | MET | A | 212 | 38.360 | 51.942 | −3.080 | 1.00 | 10.69 | A |
| ATOM | 1511 | CB | MET | A | 212 | 37.514 | 52.455 | −1.909 | 1.00 | 11.51 | A |
| ATOM | 1512 | CG | MET | A | 212 | 37.207 | 53.947 | −1.963 | 1.00 | 13.25 | A |
| ATOM | 1513 | SD | MET | A | 212 | 38.667 | 54.999 | −2.246 | 1.00 | 15.34 | A |
| ATOM | 1514 | CE | MET | A | 212 | 39.457 | 54.946 | −0.633 | 1.00 | 14.30 | A |
| ATOM | 1515 | C | MET | A | 212 | 38.741 | 50.471 | −2.866 | 1.00 | 10.61 | A |
| ATOM | 1516 | O | MET | A | 212 | 39.909 | 50.159 | −2.623 | 1.00 | 10.52 | A |
| ATOM | 1517 | N | PRO | A | 213 | 37.767 | 49.546 | −2.935 | 1.00 | 10.33 | A |
| ATOM | 1518 | CD | PRO | A | 213 | 36.300 | 49.654 | −3.018 | 1.00 | 10.77 | A |
| ATOM | 1519 | CA | PRO | A | 213 | 38.185 | 48.152 | −2.736 | 1.00 | 10.25 | A |
| ATOM | 1520 | CB | PRO | A | 213 | 36.858 | 47.377 | −2.754 | 1.00 | 10.19 | A |
| ATOM | 1521 | CG | PRO | A | 213 | 35.918 | 48.291 | −3.533 | 1.00 | 10.86 | A |
| ATOM | 1522 | C | PRO | A | 213 | 39.166 | 47.707 | −3.824 | 1.00 | 10.35 | A |
| ATOM | 1523 | O | PRO | A | 213 | 40.033 | 46.865 | −3.586 | 1.00 | 10.07 | A |
| ATOM | 1524 | N | TYR | A | 214 | 39.032 | 48.277 | −5.021 | 1.00 | 10.06 | A |
| ATOM | 1525 | CA | TYR | A | 214 | 39.937 | 47.944 | −6.120 | 1.00 | 10.18 | A |
| ATOM | 1526 | CB | TYR | A | 214 | 39.619 | 48.786 | −7.349 | 1.00 | 10.58 | A |
| ATOM | 1527 | CG | TYR | A | 214 | 40.546 | 48.531 | −8.519 | 1.00 | 11.85 | A |
| ATOM | 1528 | CD1 | TYR | A | 214 | 40.376 | 47.416 | −9.343 | 1.00 | 12.70 | A |
| ATOM | 1529 | CE1 | TYR | A | 214 | 41.207 | 47.203 | −10.446 | 1.00 | 13.49 | A |
| ATOM | 1530 | CD2 | TYR | A | 214 | 41.575 | 49.420 | −8.820 | 1.00 | 11.15 | A |
| ATOM | 1531 | CE2 | TYR | A | 214 | 42.410 | 49.215 | −9.914 | 1.00 | 11.99 | A |
| ATOM | 1532 | CZ | TYR | A | 214 | 42.220 | 48.111 | −10.726 | 1.00 | 12.95 | A |
| ATOM | 1533 | OH | TYR | A | 214 | 43.022 | 47.940 | −11.836 | 1.00 | 13.94 | A |
| ATOM | 1534 | C | TYR | A | 214 | 41.384 | 48.213 | −5.705 | 1.00 | 10.67 | A |
| ATOM | 1535 | O | TYR | A | 214 | 42.252 | 47.341 | −5.808 | 1.00 | 10.14 | A |
| ATOM | 1536 | N | ILE | A | 215 | 41.634 | 49.435 | −5.242 | 1.00 | 10.04 | A |
| ATOM | 1537 | CA | ILE | A | 215 | 42.965 | 49.848 | −4.809 | 1.00 | 10.06 | A |
| ATOM | 1538 | CB | ILE | A | 215 | 43.005 | 51.384 | −4.572 | 1.00 | 10.31 | A |
| ATOM | 1539 | CG2 | ILE | A | 215 | 44.348 | 51.795 | −3.971 | 1.00 | 10.36 | A |
| ATOM | 1540 | CG1 | ILE | A | 215 | 42.745 | 52.119 | −5.890 | 1.00 | 11.37 | A |
| ATOM | 1541 | CD1 | ILE | A | 215 | 42.716 | 53.640 | −5.753 | 1.00 | 12.28 | A |
| ATOM | 1542 | C | ILE | A | 215 | 43.399 | 49.135 | −3.523 | 1.00 | 10.17 | A |
| ATOM | 1543 | O | ILE | A | 215 | 44.529 | 48.659 | −3.413 | 1.00 | 9.97 | A |
| ATOM | 1544 | N | LEU | A | 216 | 42.497 | 49.061 | −2.552 | 1.00 | 10.00 | A |
| ATOM | 1545 | CA | LEU | A | 216 | 42.810 | 48.418 | −1.277 | 1.00 | 9.82 | A |
| ATOM | 1546 | CB | LEU | A | 216 | 41.638 | 48.595 | −0.303 | 1.00 | 9.65 | A |
| ATOM | 1547 | CG | LEU | A | 216 | 41.248 | 50.040 | 0.047 | 1.00 | 10.00 | A |
| ATOM | 1548 | CD1 | LEU | A | 216 | 39.930 | 50.050 | 0.812 | 1.00 | 9.64 | A |
| ATOM | 1549 | CD2 | LEU | A | 216 | 42.361 | 50.692 | 0.874 | 1.00 | 11.22 | A |
| ATOM | 1550 | C | LEU | A | 216 | 43.145 | 46.929 | −1.417 | 1.00 | 10.48 | A |
| ATOM | 1551 | O | LEU | A | 216 | 44.115 | 46.449 | −0.825 | 1.00 | 9.64 | A |
| ATOM | 1552 | N | GLN | A | 217 | 42.344 | 46.204 | −2.195 | 1.00 | 10.39 | A |
| ATOM | 1553 | CA | GLN | A | 217 | 42.565 | 44.773 | −2.385 | 1.00 | 11.31 | A |
| ATOM | 1554 | CB | GLN | A | 217 | 41.417 | 44.187 | −3.216 | 1.00 | 13.02 | A |
| ATOM | 1555 | CG | GLN | A | 217 | 41.367 | 42.662 | −3.309 | 1.00 | 13.54 | A |
| ATOM | 1556 | CD | GLN | A | 217 | 42.291 | 42.117 | −4.371 | 1.00 | 14.73 | A |
| ATOM | 1557 | OE1 | GLN | A | 217 | 42.492 | 42.749 | −5.406 | 1.00 | 16.14 | A |
| ATOM | 1558 | NE2 | GLN | A | 217 | 42.849 | 40.934 | −4.131 | 1.00 | 14.69 | A |
| ATOM | 1559 | C | GLN | A | 217 | 43.922 | 44.529 | −3.050 | 1.00 | 11.72 | A |
| ATOM | 1560 | O | GLN | A | 217 | 44.561 | 43.499 | −2.824 | 1.00 | 11.92 | A |
| ATOM | 1561 | N | LYS | A | 218 | 44.365 | 45.491 | −3.857 | 1.00 | 11.23 | A |
| ATOM | 1562 | CA | LYS | A | 218 | 45.655 | 45.397 | −4.537 | 1.00 | 11.32 | A |
| ATOM | 1563 | CB | LYS | A | 218 | 45.589 | 46.102 | −5.899 | 1.00 | 11.09 | A |
| ATOM | 1564 | CG | LYS | A | 218 | 44.804 | 45.324 | −6.959 | 1.00 | 12.02 | A |
| ATOM | 1565 | CD | LYS | A | 218 | 44.608 | 46.141 | −8.238 | 1.00 | 11.87 | A |
| ATOM | 1566 | CE | LYS | A | 218 | 44.232 | 45.257 | −9.421 | 1.00 | 12.02 | A |
| ATOM | 1567 | NZ | LYS | A | 218 | 43.101 | 44.326 | −9.148 | 1.00 | 12.06 | A |
| ATOM | 1568 | C | LYS | A | 218 | 46.762 | 46.019 | −3.679 | 1.00 | 11.45 | A |
| ATOM | 1569 | O | LYS | A | 218 | 47.904 | 46.154 | −4.118 | 1.00 | 11.78 | A |
| ATOM | 1570 | N | SER | A | 219 | 46.412 | 46.397 | −2.453 | 1.00 | 10.93 | A |
| ATOM | 1571 | CA | SER | A | 219 | 47.372 | 46.993 | −1.533 | 1.00 | 11.29 | A |
| ATOM | 1572 | CB | SER | A | 219 | 47.014 | 48.460 | −1.264 | 1.00 | 10.87 | A |
| ATOM | 1573 | OG | SER | A | 219 | 47.094 | 49.223 | −2.459 | 1.00 | 10.59 | A |
| ATOM | 1574 | C | SER | A | 219 | 47.434 | 46.213 | −0.219 | 1.00 | 11.22 | A |
| ATOM | 1575 | O | SER | A | 219 | 47.748 | 46.768 | 0.836 | 1.00 | 10.76 | A |
| ATOM | 1576 | N | GLY | A | 220 | 47.118 | 44.922 | −0.296 | 1.00 | 11.64 | A |
| ATOM | 1577 | CA | GLY | A | 220 | 47.176 | 44.066 | 0.878 | 1.00 | 12.01 | A |
| ATOM | 1578 | C | GLY | A | 220 | 45.984 | 44.042 | 1.820 | 1.00 | 12.11 | A |
| ATOM | 1579 | O | GLY | A | 220 | 45.995 | 43.298 | 2.801 | 1.00 | 12.08 | A |
| ATOM | 1580 | N | PHE | A | 221 | 44.947 | 44.825 | 1.541 | 1.00 | 12.32 | A |
| ATOM | 1581 | CA | PHE | A | 221 | 43.794 | 44.844 | 2.435 | 1.00 | 11.98 | A |
| ATOM | 1582 | CB | PHE | A | 221 | 42.909 | 46.065 | 2.168 | 1.00 | 12.08 | A |
| ATOM | 1583 | CG | PHE | A | 221 | 43.466 | 47.344 | 2.716 | 1.00 | 11.83 | A |
| ATOM | 1584 | CD1 | PHE | A | 221 | 44.586 | 47.935 | 2.139 | 1.00 | 10.91 | A |
| ATOM | 1585 | CD2 | PHE | A | 221 | 42.876 | 47.955 | 3.816 | 1.00 | 11.28 | A |
| ATOM | 1586 | CE1 | PHE | A | 221 | 45.111 | 49.121 | 2.651 | 1.00 | 10.48 | A |
| ATOM | 1587 | CE2 | PHE | A | 221 | 43.394 | 49.145 | 4.338 | 1.00 | 10.77 | A |
| ATOM | 1588 | CZ | PHE | A | 221 | 44.514 | 49.727 | 3.752 | 1.00 | 10.54 | A |
| ATOM | 1589 | C | PHE | A | 221 | 42.939 | 43.593 | 2.363 | 1.00 | 12.33 | A |
| ATOM | 1590 | O | PHE | A | 221 | 42.892 | 42.913 | 1.341 | 1.00 | 12.04 | A |
| ATOM | 1591 | N | LYS | A | 222 | 42.257 | 43.305 | 3.466 | 1.00 | 12.92 | A |
| ATOM | 1592 | CA | LYS | A | 222 | 41.385 | 42.147 | 3.546 | 1.00 | 14.09 | A |
| ATOM | 1593 | CB | LYS | A | 222 | 41.966 | 41.125 | 4.523 | 1.00 | 16.83 | A |
| ATOM | 1594 | CG | LYS | A | 222 | 43.173 | 40.401 | 3.950 | 1.00 | 20.43 | A |
| ATOM | 1595 | CD | LYS | A | 222 | 43.793 | 39.446 | 4.945 | 1.00 | 24.43 | A |
| ATOM | 1596 | CE | LYS | A | 222 | 44.802 | 38.537 | 4.250 | 1.00 | 25.85 | A |
| ATOM | 1597 | NZ | LYS | A | 222 | 45.771 | 39.315 | 3.420 | 1.00 | 27.78 | A |
| ATOM | 1598 | C | LYS | A | 222 | 39.974 | 42.533 | 3.966 | 1.00 | 13.76 | A |
| ATOM | 1599 | O | LYS | A | 222 | 39.043 | 41.745 | 3.804 | 1.00 | 12.76 | A |
| ATOM | 1600 | N | ASN | A | 223 | 39.819 | 43.747 | 4.492 | 1.00 | 12.50 | A |
| ATOM | 1601 | CA | ASN | A | 223 | 38.512 | 44.224 | 4.940 | 1.00 | 12.54 | A |
| ATOM | 1602 | CB | ASN | A | 223 | 38.274 | 43.836 | 6.404 | 1.00 | 12.58 | A |
| ATOM | 1603 | CG | ASN | A | 223 | 38.364 | 42.345 | 6.641 | 1.00 | 13.38 | A |
| ATOM | 1604 | OD1 | ASN | A | 223 | 39.396 | 41.832 | 7.092 | 1.00 | 14.56 | A |
| ATOM | 1605 | ND2 | ASN | A | 223 | 37.286 | 41.637 | 6.336 | 1.00 | 10.53 | A |
| ATOM | 1606 | C | ASN | A | 223 | 38.345 | 45.738 | 4.826 | 1.00 | 11.96 | A |
| ATOM | 1607 | O | ASN | A | 223 | 39.318 | 46.485 | 4.891 | 1.00 | 11.78 | A |
| ATOM | 1608 | N | MET | A | 224 | 37.101 | 46.183 | 4.668 | 1.00 | 12.00 | A |
| ATOM | 1609 | CA | MET | A | 224 | 36.800 | 47.609 | 4.598 | 1.00 | 12.07 | A |
| ATOM | 1610 | CB | MET | A | 224 | 36.915 | 48.134 | 3.165 | 1.00 | 12.02 | A |
| ATOM | 1611 | CG | MET | A | 224 | 35.849 | 47.631 | 2.207 | 1.00 | 12.35 | A |
| ATOM | 1612 | SD | MET | A | 224 | 36.063 | 48.400 | 0.596 | 1.00 | 12.43 | A |
| ATOM | 1613 | CE | MET | A | 224 | 35.436 | 50.044 | 0.920 | 1.00 | 12.22 | A |
| ATOM | 1614 | C | MET | A | 224 | 35.402 | 47.892 | 5.142 | 1.00 | 12.41 | A |
| ATOM | 1615 | O | MET | A | 224 | 34.516 | 47.029 | 5.113 | 1.00 | 11.89 | A |
| ATOM | 1616 | N | LEU | A | 225 | 35.221 | 49.106 | 5.649 | 1.00 | 11.22 | A |
| ATOM | 1617 | CA | LEU | A | 225 | 33.950 | 49.530 | 6.219 | 1.00 | 11.40 | A |
| ATOM | 1618 | CB | LEU | A | 225 | 34.090 | 49.648 | 7.738 | 1.00 | 10.91 | A |
| ATOM | 1619 | CG | LEU | A | 225 | 32.929 | 50.260 | 8.531 | 1.00 | 11.65 | A |
| ATOM | 1620 | CD1 | LEU | A | 225 | 32.932 | 49.685 | 9.935 | 1.00 | 11.52 | A |
| ATOM | 1621 | CD2 | LEU | A | 225 | 33.046 | 51.798 | 8.559 | 1.00 | 11.31 | A |
| ATOM | 1622 | C | LEU | A | 225 | 33.525 | 50.868 | 5.626 | 1.00 | 11.04 | A |
| ATOM | 1623 | O | LEU | A | 225 | 34.351 | 51.762 | 5.451 | 1.00 | 10.88 | A |
| ATOM | 1624 | N | ILE | A | 226 | 32.237 | 50.997 | 5.315 | 1.00 | 11.47 | A |
| ATOM | 1625 | CA | ILE | A | 226 | 31.699 | 52.232 | 4.747 | 1.00 | 11.81 | A |
| ATOM | 1626 | CB | ILE | A | 226 | 31.371 | 52.059 | 3.242 | 1.00 | 11.66 | A |
| ATOM | 1627 | CG2 | ILE | A | 226 | 32.645 | 51.699 | 2.478 | 1.00 | 11.16 | A |
| ATOM | 1628 | CG1 | ILE | A | 226 | 30.315 | 50.968 | 3.048 | 1.00 | 11.34 | A |
| ATOM | 1629 | CD1 | ILE | A | 226 | 29.894 | 50.771 | 1.596 | 1.00 | 11.84 | A |
| ATOM | 1630 | C | ILE | A | 226 | 30.441 | 52.632 | 5.516 | 1.00 | 12.31 | A |
| ATOM | 1631 | O | ILE | A | 226 | 29.856 | 51.805 | 6.222 | 1.00 | 12.85 | A |
| ATOM | 1632 | N | GLN | A | 227 | 30.020 | 53.888 | 5.381 | 1.00 | 12.34 | A |
| ATOM | 1633 | CA | GLN | A | 227 | 28.853 | 54.367 | 6.118 | 1.00 | 12.23 | A |
| ATOM | 1634 | CB | GLN | A | 227 | 29.334 | 55.170 | 7.334 | 1.00 | 12.52 | A |
| ATOM | 1635 | CG | GLN | A | 227 | 28.376 | 56.253 | 7.845 | 1.00 | 12.98 | A |
| ATOM | 1636 | CD | GLN | A | 227 | 27.053 | 55.713 | 8.357 | 1.00 | 13.37 | A |
| ATOM | 1637 | OE1 | GLN | A | 227 | 26.951 | 54.552 | 8.753 | 1.00 | 14.02 | A |
| ATOM | 1638 | NE2 | GLN | A | 227 | 26.034 | 56.568 | 8.375 | 1.00 | 12.44 | A |
| ATOM | 1639 | C | GLN | A | 227 | 27.814 | 55.187 | 5.351 | 1.00 | 12.86 | A |
| ATOM | 1640 | O | GLN | A | 227 | 26.618 | 54.888 | 5.408 | 1.00 | 12.43 | A |
| ATOM | 1641 | N | ARG | A | 228 | 28.252 | 56.225 | 4.645 | 1.00 | 12.28 | A |
| ATOM | 1642 | CA | ARG | A | 228 | 27.303 | 57.070 | 3.941 | 1.00 | 12.28 | A |
| ATOM | 1643 | CB | ARG | A | 228 | 27.893 | 58.470 | 3.726 | 1.00 | 12.59 | A |
| ATOM | 1644 | CG | ARG | A | 228 | 28.063 | 59.258 | 5.022 | 1.00 | 12.49 | A |
| ATOM | 1645 | CD | ARG | A | 228 | 28.404 | 60.729 | 4.772 | 1.00 | 12.20 | A |
| ATOM | 1646 | NE | ARG | A | 228 | 28.640 | 61.461 | 6.022 | 1.00 | 12.35 | A |
| ATOM | 1647 | CZ | ARG | A | 228 | 27.683 | 61.920 | 6.828 | 1.00 | 14.14 | A |
| ATOM | 1648 | NH1 | ARG | A | 228 | 26.400 | 61.736 | 6.525 | 1.00 | 14.04 | A |
| ATOM | 1649 | NH2 | ARG | A | 228 | 28.007 | 62.554 | 7.951 | 1.00 | 13.35 | A |
| ATOM | 1650 | C | ARG | A | 228 | 26.759 | 56.523 | 2.628 | 1.00 | 12.67 | A |
| ATOM | 1651 | O | ARG | A | 228 | 27.323 | 56.744 | 1.557 | 1.00 | 12.03 | A |
| ATOM | 1652 | N | THR | A | 229 | 25.653 | 55.795 | 2.735 | 1.00 | 12.60 | A |
| ATOM | 1653 | CA | THR | A | 229 | 24.976 | 55.240 | 1.573 | 1.00 | 12.10 | A |
| ATOM | 1654 | CB | THR | A | 229 | 25.136 | 53.695 | 1.490 | 1.00 | 12.81 | A |
| ATOM | 1655 | OG1 | THR | A | 229 | 24.559 | 53.075 | 2.648 | 1.00 | 12.19 | A |
| ATOM | 1656 | CG2 | THR | A | 229 | 26.617 | 53.324 | 1.405 | 1.00 | 11.20 | A |
| ATOM | 1657 | C | THR | A | 229 | 23.506 | 55.619 | 1.729 | 1.00 | 12.60 | A |
| ATOM | 1658 | O | THR | A | 229 | 23.035 | 55.849 | 2.848 | 1.00 | 13.73 | A |
| ATOM | 1659 | N | HIS | A | 230 | 22.796 | 55.701 | 0.608 | 1.00 | 12.44 | A |
| ATOM | 1660 | CA | HIS | A | 230 | 21.380 | 56.080 | 0.589 | 1.00 | 12.74 | A |
| ATOM | 1661 | CB | HIS | A | 230 | 20.803 | 55.794 | −0.803 | 1.00 | 13.25 | A |
| ATOM | 1662 | CG | HIS | A | 230 | 19.545 | 56.546 | −1.111 | 1.00 | 13.82 | A |
| ATOM | 1663 | CD2 | HIS | A | 230 | 19.268 | 57.462 | −2.070 | 1.00 | 14.52 | A |
| ATOM | 1664 | ND1 | HIS | A | 230 | 18.382 | 56.384 | −0.389 | 1.00 | 13.28 | A |
| ATOM | 1665 | CE1 | HIS | A | 230 | 17.443 | 57.169 | −0.889 | 1.00 | 13.41 | A |
| ATOM | 1666 | NE2 | HIS | A | 230 | 17.954 | 57.834 | −1.910 | 1.00 | 12.86 | A |
| ATOM | 1667 | C | HIS | A | 230 | 20.570 | 55.343 | 1.656 | 1.00 | 12.53 | A |
| ATOM | 1668 | O | HIS | A | 230 | 20.672 | 54.125 | 1.788 | 1.00 | 12.07 | A |
| ATOM | 1669 | N | TYR | A | 231 | 19.756 | 56.080 | 2.413 | 1.00 | 12.70 | A |
| ATOM | 1670 | CA | TYR | A | 231 | 18.958 | 55.453 | 3.463 | 1.00 | 13.53 | A |
| ATOM | 1671 | CB | TYR | A | 231 | 18.105 | 56.499 | 4.200 | 1.00 | 13.42 | A |
| ATOM | 1672 | CG | TYR | A | 231 | 17.122 | 57.268 | 3.337 | 1.00 | 14.84 | A |
| ATOM | 1673 | CD1 | TYR | A | 231 | 15.840 | 56.772 | 3.088 | 1.00 | 14.12 | A |
| ATOM | 1674 | CE1 | TYR | A | 231 | 14.934 | 57.484 | 2.305 | 1.00 | 14.51 | A |
| ATOM | 1675 | CD2 | TYR | A | 231 | 17.474 | 58.500 | 2.776 | 1.00 | 14.31 | A |
| ATOM | 1676 | CE2 | TYR | A | 231 | 16.576 | 59.217 | 1.992 | 1.00 | 14.26 | A |
| ATOM | 1677 | CZ | TYR | A | 231 | 15.308 | 58.704 | 1.761 | 1.00 | 14.26 | A |
| ATOM | 1678 | OH | TYR | A | 231 | 14.421 | 59.409 | 0.982 | 1.00 | 14.93 | A |
| ATOM | 1679 | C | TYR | A | 231 | 18.081 | 54.316 | 2.936 | 1.00 | 14.05 | A |
| ATOM | 1680 | O | TYR | A | 231 | 17.785 | 53.368 | 3.661 | 1.00 | 14.36 | A |
| ATOM | 1681 | N | SER | A | 232 | 17.675 | 54.399 | 1.674 | 1.00 | 14.47 | A |
| ATOM | 1682 | CA | SER | A | 232 | 16.847 | 53.348 | 1.086 | 1.00 | 15.06 | A |
| ATOM | 1683 | CB | SER | A | 232 | 16.235 | 53.823 | −0.233 | 1.00 | 15.79 | A |
| ATOM | 1684 | OG | SER | A | 232 | 15.246 | 54.811 | −0.004 | 1.00 | 17.41 | A |
| ATOM | 1685 | C | SER | A | 232 | 17.650 | 52.071 | 0.854 | 1.00 | 14.95 | A |
| ATOM | 1686 | O | SER | A | 232 | 17.120 | 50.966 | 0.967 | 1.00 | 13.88 | A |
| ATOM | 1687 | N | VAL | A | 233 | 18.931 | 52.229 | 0.529 | 1.00 | 14.34 | A |
| ATOM | 1688 | CA | VAL | A | 233 | 19.807 | 51.085 | 0.295 | 1.00 | 13.73 | A |
| ATOM | 1689 | CB | VAL | A | 233 | 21.136 | 51.534 | −0.355 | 1.00 | 13.36 | A |
| ATOM | 1690 | CG1 | VAL | A | 233 | 22.122 | 50.372 | −0.397 | 1.00 | 13.01 | A |
| ATOM | 1691 | CG2 | VAL | A | 233 | 20.868 | 52.043 | −1.769 | 1.00 | 13.28 | A |
| ATOM | 1692 | C | VAL | A | 233 | 20.091 | 50.364 | 1.616 | 1.00 | 13.86 | A |
| ATOM | 1693 | O | VAL | A | 233 | 20.086 | 49.131 | 1.679 | 1.00 | 13.64 | A |
| ATOM | 1694 | N | LYS | A | 234 | 20.329 | 51.137 | 2.668 | 1.00 | 13.48 | A |
| ATOM | 1695 | CA | LYS | A | 234 | 20.583 | 50.569 | 3.987 | 1.00 | 14.03 | A |
| ATOM | 1696 | CB | LYS | A | 234 | 20.800 | 51.689 | 5.013 | 1.00 | 14.30 | A |
| ATOM | 1697 | CG | LYS | A | 234 | 22.142 | 52.414 | 4.890 | 1.00 | 14.69 | A |
| ATOM | 1698 | CD | LYS | A | 234 | 22.193 | 53.642 | 5.799 | 1.00 | 13.66 | A |
| ATOM | 1699 | CE | LYS | A | 234 | 23.563 | 54.329 | 5.762 | 1.00 | 13.23 | A |
| ATOM | 1700 | NZ | LYS | A | 234 | 24.575 | 53.699 | 6.669 | 1.00 | 12.68 | A |
| ATOM | 1701 | C | LYS | A | 234 | 19.392 | 49.706 | 4.405 | 1.00 | 14.10 | A |
| ATOM | 1702 | O | LYS | A | 234 | 19.563 | 48.582 | 4.877 | 1.00 | 13.22 | A |
| ATOM | 1703 | N | LYS | A | 235 | 18.186 | 50.233 | 4.218 | 1.00 | 15.31 | A |
| ATOM | 1704 | CA | LYS | A | 235 | 16.973 | 49.502 | 4.584 | 1.00 | 16.19 | A |
| ATOM | 1705 | CB | LYS | A | 235 | 15.739 | 50.378 | 4.359 | 1.00 | 17.38 | A |
| ATOM | 1706 | CG | LYS | A | 235 | 14.446 | 49.778 | 4.897 | 1.00 | 17.88 | A |
| ATOM | 1707 | CD | LYS | A | 235 | 13.270 | 50.694 | 4.617 | 1.00 | 18.37 | A |
| ATOM | 1708 | CE | LYS | A | 235 | 11.979 | 50.115 | 5.171 | 1.00 | 20.00 | A |
| ATOM | 1709 | NZ | LYS | A | 235 | 10.809 | 50.963 | 4.811 | 1.00 | 20.45 | A |
| ATOM | 1710 | C | LYS | A | 235 | 16.846 | 48.212 | 3.780 | 1.00 | 16.38 | A |
| ATOM | 1711 | O | LYS | A | 235 | 16.594 | 47.143 | 4.339 | 1.00 | 16.89 | A |
| ATOM | 1712 | N | GLU | A | 236 | 17.031 | 48.314 | 2.469 | 1.00 | 17.01 | A |
| ATOM | 1713 | CA | GLU | A | 236 | 16.933 | 47.157 | 1.583 | 1.00 | 17.20 | A |
| ATOM | 1714 | CB | GLU | A | 236 | 17.130 | 47.599 | 0.129 | 1.00 | 19.36 | A |
| ATOM | 1715 | CG | GLU | A | 236 | 16.943 | 46.487 | −0.894 | 1.00 | 22.75 | A |
| ATOM | 1716 | CD | GLU | A | 236 | 15.493 | 46.037 | −1.033 | 1.00 | 24.36 | A |
| ATOM | 1717 | OE1 | GLU | A | 236 | 15.237 | 45.125 | −1.844 | 1.00 | 26.15 | A |
| ATOM | 1718 | OE2 | GLU | A | 236 | 14.610 | 46.592 | −0.341 | 1.00 | 25.45 | A |
| ATOM | 1719 | C | GLU | A | 236 | 17.946 | 46.063 | 1.931 | 1.00 | 16.81 | A |
| ATOM | 1720 | O | GLU | A | 236 | 17.585 | 44.895 | 2.078 | 1.00 | 16.28 | A |
| ATOM | 1721 | N | LEU | A | 237 | 19.216 | 46.433 | 2.064 | 1.00 | 15.97 | A |
| ATOM | 1722 | CA | LEU | A | 237 | 20.238 | 45.447 | 2.387 | 1.00 | 15.97 | A |
| ATOM | 1723 | CB | LEU | A | 237 | 21.635 | 46.048 | 2.220 | 1.00 | 15.23 | A |
| ATOM | 1724 | CG | LEU | A | 237 | 21.976 | 46.514 | 0.799 | 1.00 | 15.74 | A |
| ATOM | 1725 | CD1 | LEU | A | 237 | 23.356 | 47.142 | 0.797 | 1.00 | 16.24 | A |
| ATOM | 1726 | CD2 | LEU | A | 237 | 21.919 | 45.337 | −0.173 | 1.00 | 16.31 | A |
| ATOM | 1727 | C | LEU | A | 237 | 20.066 | 44.898 | 3.800 | 1.00 | 15.86 | A |
| ATOM | 1728 | O | LEU | A | 237 | 20.324 | 43.719 | 4.047 | 1.00 | 16.07 | A |
| ATOM | 1729 | N | ALA | A | 238 | 19.624 | 45.743 | 4.725 | 1.00 | 15.71 | A |
| ATOM | 1730 | CA | ALA | A | 238 | 19.420 | 45.302 | 6.099 | 1.00 | 16.72 | A |
| ATOM | 1731 | CB | ALA | A | 238 | 18.976 | 46.473 | 6.968 | 1.00 | 14.76 | A |
| ATOM | 1732 | C | ALA | A | 238 | 18.373 | 44.187 | 6.149 | 1.00 | 17.50 | A |
| ATOM | 1733 | O | ALA | A | 238 | 18.559 | 43.177 | 6.832 | 1.00 | 17.31 | A |
| ATOM | 1734 | N | GLN | A | 239 | 17.279 | 44.374 | 5.418 | 1.00 | 18.24 | A |
| ATOM | 1735 | CA | GLN | A | 239 | 16.199 | 43.391 | 5.394 | 1.00 | 19.52 | A |
| ATOM | 1736 | CB | GLN | A | 239 | 15.031 | 43.915 | 4.550 | 1.00 | 20.50 | A |
| ATOM | 1737 | CG | GLN | A | 239 | 14.442 | 45.225 | 5.068 | 1.00 | 23.60 | A |
| ATOM | 1738 | CD | GLN | A | 239 | 13.275 | 45.728 | 4.235 | 1.00 | 25.24 | A |
| ATOM | 1739 | OE1 | GLN | A | 239 | 13.364 | 45.819 | 3.009 | 1.00 | 27.04 | A |
| ATOM | 1740 | NE2 | GLN | A | 239 | 12.175 | 46.069 | 4.900 | 1.00 | 27.03 | A |
| ATOM | 1741 | C | GLN | A | 239 | 16.652 | 42.028 | 4.872 | 1.00 | 19.61 | A |
| ATOM | 1742 | O | GLN | A | 239 | 16.080 | 41.000 | 5.230 | 1.00 | 19.90 | A |
| ATOM | 1743 | N | GLN | A | 240 | 17.679 | 42.018 | 4.029 | 1.00 | 19.18 | A |
| ATOM | 1744 | CA | GLN | A | 240 | 18.189 | 40.768 | 3.472 | 1.00 | 18.91 | A |
| ATOM | 1745 | CB | GLN | A | 240 | 18.421 | 40.927 | 1.969 | 1.00 | 21.14 | A |
| ATOM | 1746 | CG | GLN | A | 240 | 17.200 | 41.421 | 1.212 | 1.00 | 23.97 | A |
| ATOM | 1747 | CD | GLN | A | 240 | 16.065 | 40.422 | 1.230 | 1.00 | 25.55 | A |
| ATOM | 1748 | OE1 | GLN | A | 240 | 14.910 | 40.777 | 0.998 | 1.00 | 28.30 | A |
| ATOM | 1749 | NE2 | GLN | A | 240 | 16.387 | 39.160 | 1.494 | 1.00 | 26.14 | A |
| ATOM | 1750 | C | GLN | A | 240 | 19.495 | 40.350 | 4.139 | 1.00 | 17.92 | A |
| ATOM | 1751 | O | GLN | A | 240 | 20.113 | 39.364 | 3.737 | 1.00 | 16.97 | A |
| ATOM | 1752 | N | ARG | A | 241 | 19.895 | 41.090 | 5.171 | 1.00 | 16.88 | A |
| ATOM | 1753 | CA | ARG | A | 241 | 21.149 | 40.831 | 5.871 | 1.00 | 16.09 | A |
| ATOM | 1754 | CB | ARG | A | 241 | 21.084 | 39.521 | 6.668 | 1.00 | 17.31 | A |
| ATOM | 1755 | CG | ARG | A | 241 | 20.052 | 39.549 | 7.792 | 1.00 | 18.25 | A |
| ATOM | 1756 | CD | ARG | A | 241 | 20.258 | 38.407 | 8.776 | 1.00 | 19.71 | A |
| ATOM | 1757 | NE | ARG | A | 241 | 20.252 | 37.106 | 8.114 | 1.00 | 21.50 | A |
| ATOM | 1758 | CZ | ARG | A | 241 | 20.610 | 35.966 | 8.700 | 1.00 | 23.01 | A |
| ATOM | 1759 | NH1 | ARG | A | 241 | 21.004 | 35.962 | 9.969 | 1.00 | 23.30 | A |
| ATOM | 1760 | NH2 | ARG | A | 241 | 20.583 | 34.831 | 8.014 | 1.00 | 23.18 | A |
| ATOM | 1761 | C | ARG | A | 241 | 22.284 | 40.784 | 4.853 | 1.00 | 15.76 | A |
| ATOM | 1762 | O | ARG | A | 241 | 23.092 | 39.850 | 4.824 | 1.00 | 14.26 | A |
| ATOM | 1763 | N | GLN | A | 242 | 22.327 | 41.807 | 4.004 | 1.00 | 15.05 | A |
| ATOM | 1764 | CA | GLN | A | 242 | 23.360 | 41.912 | 2.979 | 1.00 | 14.63 | A |
| ATOM | 1765 | CB | GLN | A | 242 | 22.721 | 41.946 | 1.584 | 1.00 | 14.76 | A |
| ATOM | 1766 | CG | GLN | A | 242 | 21.909 | 40.704 | 1.233 | 1.00 | 15.30 | A |
| ATOM | 1767 | CD | GLN | A | 242 | 21.188 | 40.836 | −0.096 | 1.00 | 15.32 | A |
| ATOM | 1768 | OE1 | GLN | A | 242 | 20.643 | 41.892 | −0.411 | 1.00 | 15.92 | A |
| ATOM | 1769 | NE2 | GLN | A | 242 | 21.170 | 39.759 | −0.878 | 1.00 | 15.08 | A |
| ATOM | 1770 | C | GLN | A | 242 | 24.197 | 43.175 | 3.198 | 1.00 | 14.54 | A |
| ATOM | 1771 | O | GLN | A | 242 | 24.712 | 43.753 | 2.242 | 1.00 | 13.67 | A |
| ATOM | 1772 | N | LEU | A | 243 | 24.323 | 43.594 | 4.458 | 1.00 | 13.82 | A |
| ATOM | 1773 | CA | LEU | A | 243 | 25.106 | 44.779 | 4.808 | 1.00 | 14.57 | A |
| ATOM | 1774 | CB | LEU | A | 243 | 24.690 | 45.307 | 6.185 | 1.00 | 14.76 | A |
| ATOM | 1775 | CG | LEU | A | 243 | 23.302 | 45.958 | 6.227 | 1.00 | 16.04 | A |
| ATOM | 1776 | CD1 | LEU | A | 243 | 22.895 | 46.231 | 7.673 | 1.00 | 16.58 | A |
| ATOM | 1777 | CD2 | LEU | A | 243 | 23.321 | 47.258 | 5.413 | 1.00 | 16.24 | A |
| ATOM | 1778 | C | LEU | A | 243 | 26.598 | 44.455 | 4.786 | 1.00 | 14.27 | A |
| ATOM | 1779 | O | LEU | A | 243 | 27.440 | 45.352 | 4.712 | 1.00 | 14.53 | A |
| ATOM | 1780 | N | GLU | A | 244 | 26.918 | 43.168 | 4.875 | 1.00 | 13.75 | A |
| ATOM | 1781 | CA | GLU | A | 244 | 28.302 | 42.720 | 4.789 | 1.00 | 13.31 | A |
| ATOM | 1782 | CB | GLU | A | 244 | 28.702 | 41.897 | 6.017 | 1.00 | 13.04 | A |
| ATOM | 1783 | CG | GLU | A | 244 | 28.891 | 42.777 | 7.249 | 1.00 | 12.64 | A |
| ATOM | 1784 | CD | GLU | A | 244 | 29.323 | 42.010 | 8.476 | 1.00 | 13.11 | A |
| ATOM | 1785 | OE1 | GLU | A | 244 | 28.806 | 40.897 | 8.699 | 1.00 | 12.89 | A |
| ATOM | 1786 | OE2 | GLU | A | 244 | 30.173 | 42.532 | 9.225 | 1.00 | 13.62 | A |
| ATOM | 1787 | C | GLU | A | 244 | 28.326 | 41.893 | 3.522 | 1.00 | 13.46 | A |
| ATOM | 1788 | O | GLU | A | 244 | 27.505 | 40.992 | 3.344 | 1.00 | 13.61 | A |
| ATOM | 1789 | N | PHE | A | 245 | 29.256 | 42.215 | 2.631 | 1.00 | 13.16 | A |
| ATOM | 1790 | CA | PHE | A | 245 | 29.330 | 41.534 | 1.349 | 1.00 | 12.82 | A |
| ATOM | 1791 | CB | PHE | A | 245 | 28.365 | 42.226 | 0.379 | 1.00 | 12.71 | A |
| ATOM | 1792 | CG | PHE | A | 245 | 28.495 | 43.731 | 0.367 | 1.00 | 13.34 | A |
| ATOM | 1793 | CD1 | PHE | A | 245 | 29.477 | 44.358 | −0.396 | 1.00 | 13.26 | A |
| ATOM | 1794 | CD2 | PHE | A | 245 | 27.653 | 44.518 | 1.152 | 1.00 | 13.80 | A |
| ATOM | 1795 | CE1 | PHE | A | 245 | 29.620 | 45.761 | −0.377 | 1.00 | 14.25 | A |
| ATOM | 1796 | CE2 | PHE | A | 245 | 27.785 | 45.915 | 1.181 | 1.00 | 13.45 | A |
| ATOM | 1797 | CZ | PHE | A | 245 | 28.771 | 46.536 | 0.416 | 1.00 | 13.39 | A |
| ATOM | 1798 | C | PHE | A | 245 | 30.732 | 41.529 | 0.759 | 1.00 | 12.74 | A |
| ATOM | 1799 | O | PHE | A | 245 | 31.610 | 42.268 | 1.202 | 1.00 | 12.98 | A |
| ATOM | 1800 | N | LEU | A | 246 | 30.934 | 40.676 | −0.239 | 1.00 | 12.93 | A |
| ATOM | 1801 | CA | LEU | A | 246 | 32.209 | 40.590 | −0.931 | 1.00 | 12.80 | A |
| ATOM | 1802 | CB | LEU | A | 246 | 32.480 | 39.143 | −1.352 | 1.00 | 14.15 | A |
| ATOM | 1803 | CG | LEU | A | 246 | 32.798 | 38.226 | −0.162 | 1.00 | 15.44 | A |
| ATOM | 1804 | CD1 | LEU | A | 246 | 32.721 | 36.755 | −0.566 | 1.00 | 16.21 | A |
| ATOM | 1805 | CD2 | LEU | A | 246 | 34.186 | 38.567 | 0.356 | 1.00 | 16.84 | A |
| ATOM | 1806 | C | LEU | A | 246 | 32.062 | 41.506 | −2.140 | 1.00 | 12.88 | A |
| ATOM | 1807 | O | LEU | A | 246 | 31.450 | 41.142 | −3.143 | 1.00 | 12.86 | A |
| ATOM | 1808 | N | TRP | A | 247 | 32.616 | 42.709 | −2.021 | 1.00 | 12.15 | A |
| ATOM | 1809 | CA | TRP | A | 247 | 32.528 | 43.725 | −3.064 | 1.00 | 10.93 | A |
| ATOM | 1810 | CB | TRP | A | 247 | 32.730 | 45.109 | −2.428 | 1.00 | 11.30 | A |
| ATOM | 1811 | CG | TRP | A | 247 | 32.227 | 46.275 | −3.234 | 1.00 | 10.64 | A |
| ATOM | 1812 | CD2 | TRP | A | 247 | 32.231 | 47.653 | −2.835 | 1.00 | 10.92 | A |
| ATOM | 1813 | CE2 | TRP | A | 247 | 31.620 | 48.394 | −3.875 | 1.00 | 10.68 | A |
| ATOM | 1814 | CE3 | TRP | A | 247 | 32.690 | 48.333 | −1.699 | 1.00 | 10.84 | A |
| ATOM | 1815 | CD1 | TRP | A | 247 | 31.638 | 46.237 | −4.465 | 1.00 | 11.11 | A |
| ATOM | 1816 | NE1 | TRP | A | 247 | 31.267 | 47.509 | −4.858 | 1.00 | 10.24 | A |
| ATOM | 1817 | CZ2 | TRP | A | 247 | 31.455 | 49.783 | −3.809 | 1.00 | 11.19 | A |
| ATOM | 1818 | CZ3 | TRP | A | 247 | 32.527 | 49.715 | −1.635 | 1.00 | 10.19 | A |
| ATOM | 1819 | CH2 | TRP | A | 247 | 31.914 | 50.424 | −2.684 | 1.00 | 10.43 | A |
| ATOM | 1820 | C | TRP | A | 247 | 33.547 | 43.517 | −4.181 | 1.00 | 11.11 | A |
| ATOM | 1821 | O | TRP | A | 247 | 34.749 | 43.713 | −3.979 | 1.00 | 11.48 | A |
| ATOM | 1822 | N | ARG | A | 248 | 33.062 | 43.116 | −5.355 | 1.00 | 10.75 | A |
| ATOM | 1823 | CA | ARG | A | 248 | 33.929 | 42.905 | −6.509 | 1.00 | 10.77 | A |
| ATOM | 1824 | CB | ARG | A | 248 | 33.699 | 41.518 | −7.123 | 1.00 | 11.28 | A |
| ATOM | 1825 | CG | ARG | A | 248 | 32.347 | 41.360 | −7.812 | 1.00 | 12.39 | A |
| ATOM | 1826 | CD | ARG | A | 248 | 32.287 | 40.064 | −8.622 | 1.00 | 12.96 | A |
| ATOM | 1827 | NE | ARG | A | 248 | 32.307 | 38.877 | −7.771 | 1.00 | 13.78 | A |
| ATOM | 1828 | CZ | ARG | A | 248 | 32.297 | 37.627 | −8.232 | 1.00 | 15.01 | A |
| ATOM | 1829 | NH1 | ARG | A | 248 | 32.272 | 37.396 | −9.540 | 1.00 | 14.24 | A |
| ATOM | 1830 | NH2 | ARG | A | 248 | 32.303 | 36.603 | −7.387 | 1.00 | 15.39 | A |
| ATOM | 1831 | C | ARG | A | 248 | 33.618 | 43.973 | −7.557 | 1.00 | 10.84 | A |
| ATOM | 1832 | O | ARG | A | 248 | 32.565 | 44.613 | −7.508 | 1.00 | 10.46 | A |
| ATOM | 1833 | N | GLN | A | 249 | 34.531 | 44.154 | −8.504 | 1.00 | 10.83 | A |
| ATOM | 1834 | CA | GLN | A | 249 | 34.351 | 45.141 | −9.560 | 1.00 | 11.65 | A |
| ATOM | 1835 | CB | GLN | A | 249 | 35.678 | 45.363 | −10.298 | 1.00 | 11.30 | A |
| ATOM | 1836 | CG | GLN | A | 249 | 36.810 | 45.827 | −9.377 | 1.00 | 11.05 | A |
| ATOM | 1837 | CD | GLN | A | 249 | 36.444 | 47.085 | −8.598 | 1.00 | 11.05 | A |
| ATOM | 1838 | OE1 | GLN | A | 249 | 36.457 | 47.098 | −7.361 | 1.00 | 12.77 | A |
| ATOM | 1839 | NE2 | GLN | A | 249 | 36.111 | 48.148 | −9.320 | 1.00 | 9.23 | A |
| ATOM | 1840 | C | GLN | A | 249 | 33.256 | 44.688 | −10.524 | 1.00 | 12.10 | A |
| ATOM | 1841 | O | GLN | A | 249 | 33.049 | 43.490 | −10.725 | 1.00 | 11.94 | A |
| ATOM | 1842 | N | ILE | A | 250 | 32.553 | 45.647 | −11.122 | 1.00 | 12.93 | A |
| ATOM | 1843 | CA | ILE | A | 250 | 31.453 | 45.320 | −12.026 | 1.00 | 13.51 | A |
| ATOM | 1844 | CB | ILE | A | 250 | 30.767 | 46.602 | −12.572 | 1.00 | 13.77 | A |
| ATOM | 1845 | CG2 | ILE | A | 250 | 30.103 | 47.363 | −11.428 | 1.00 | 13.59 | A |
| ATOM | 1846 | CG1 | ILE | A | 250 | 31.785 | 47.485 | −13.296 | 1.00 | 13.49 | A |
| ATOM | 1847 | CD1 | ILE | A | 250 | 31.191 | 48.781 | −13.837 | 1.00 | 14.87 | A |
| ATOM | 1848 | C | ILE | A | 250 | 31.782 | 44.404 | −13.208 | 1.00 | 14.67 | A |
| ATOM | 1849 | O | ILE | A | 250 | 30.896 | 43.715 | −13.721 | 1.00 | 14.61 | A |
| ATOM | 1850 | N | TRP | A | 251 | 33.043 | 44.376 | −13.626 | 1.00 | 15.25 | A |
| ATOM | 1851 | CA | TRP | A | 251 | 33.455 | 43.547 | −14.761 | 1.00 | 16.73 | A |
| ATOM | 1852 | CB | TRP | A | 251 | 34.444 | 44.325 | −15.625 | 1.00 | 16.94 | A |
| ATOM | 1853 | CG | TRP | A | 251 | 35.745 | 44.514 | −14.923 | 1.00 | 17.70 | A |
| ATOM | 1854 | CD2 | TRP | A | 251 | 36.159 | 45.669 | −14.196 | 1.00 | 17.07 | A |
| ATOM | 1855 | CE2 | TRP | A | 251 | 37.412 | 45.372 | −13.618 | 1.00 | 17.86 | A |
| ATOM | 1856 | CE3 | TRP | A | 251 | 35.591 | 46.929 | −13.974 | 1.00 | 17.14 | A |
| ATOM | 1857 | CD1 | TRP | A | 251 | 36.738 | 43.585 | −14.765 | 1.00 | 17.86 | A |
| ATOM | 1858 | NE1 | TRP | A | 251 | 37.740 | 44.091 | −13.981 | 1.00 | 17.65 | A |
| ATOM | 1859 | CZ2 | TRP | A | 251 | 38.108 | 46.289 | −12.830 | 1.00 | 17.71 | A |
| ATOM | 1860 | CZ3 | TRP | A | 251 | 36.282 | 47.842 | −13.191 | 1.00 | 17.37 | A |
| ATOM | 1861 | CH2 | TRP | A | 251 | 37.529 | 47.516 | −12.628 | 1.00 | 18.49 | A |
| ATOM | 1862 | C | TRP | A | 251 | 34.121 | 42.234 | −14.344 | 1.00 | 18.00 | A |
| ATOM | 1863 | O | TRP | A | 251 | 34.454 | 41.403 | −15.194 | 1.00 | 18.22 | A |
| ATOM | 1864 | N | ASP | A | 252 | 34.325 | 42.062 | −13.043 | 1.00 | 18.23 | A |
| ATOM | 1865 | CA | ASP | A | 252 | 34.996 | 40.883 | −12.499 | 1.00 | 19.34 | A |
| ATOM | 1866 | CB | ASP | A | 252 | 35.549 | 41.228 | −11.110 | 1.00 | 18.94 | A |
| ATOM | 1867 | CG | ASP | A | 252 | 36.320 | 40.084 | −10.484 | 1.00 | 20.28 | A |
| ATOM | 1868 | OD1 | ASP | A | 252 | 36.654 | 39.117 | −11.207 | 1.00 | 20.52 | A |
| ATOM | 1869 | OD2 | ASP | A | 252 | 36.596 | 40.161 | −9.267 | 1.00 | 18.06 | A |
| ATOM | 1870 | C | ASP | A | 252 | 34.121 | 39.630 | −12.436 | 1.00 | 19.79 | A |
| ATOM | 1871 | O | ASP | A | 252 | 33.323 | 39.456 | −11.520 | 1.00 | 19.33 | A |
| ATOM | 1872 | N | ASN | A | 253 | 34.292 | 38.750 | −13.417 | 1.00 | 21.32 | A |
| ATOM | 1873 | CA | ASN | A | 253 | 33.513 | 37.520 | −13.487 | 1.00 | 22.54 | A |
| ATOM | 1874 | CB | ASN | A | 253 | 33.580 | 36.954 | −14.907 | 1.00 | 24.16 | A |
| ATOM | 1875 | CG | ASN | A | 253 | 32.717 | 35.727 | −15.085 | 1.00 | 25.52 | A |
| ATOM | 1876 | OD1 | ASN | A | 253 | 33.192 | 34.684 | −15.530 | 1.00 | 26.98 | A |
| ATOM | 1877 | ND2 | ASN | A | 253 | 31.439 | 35.845 | −14.744 | 1.00 | 25.84 | A |
| ATOM | 1878 | C | ASN | A | 253 | 33.980 | 36.456 | −12.490 | 1.00 | 22.75 | A |
| ATOM | 1879 | O | ASN | A | 253 | 33.162 | 35.747 | −11.903 | 1.00 | 23.28 | A |
| ATOM | 1880 | N | LYS | A | 254 | 35.290 | 36.355 | −12.295 | 1.00 | 23.59 | A |
| ATOM | 1881 | CA | LYS | A | 254 | 35.855 | 35.357 | −11.385 | 1.00 | 24.65 | A |
| ATOM | 1882 | CB | LYS | A | 254 | 37.324 | 35.101 | −11.744 | 1.00 | 26.71 | A |
| ATOM | 1883 | CG | LYS | A | 254 | 37.939 | 33.877 | −11.070 | 1.00 | 28.98 | A |
| ATOM | 1884 | CD | LYS | A | 254 | 39.324 | 33.578 | −11.640 | 1.00 | 30.99 | A |
| ATOM | 1885 | CE | LYS | A | 254 | 39.916 | 32.299 | −11.055 | 1.00 | 31.41 | A |
| ATOM | 1886 | NZ | LYS | A | 254 | 40.115 | 32.383 | −9.580 | 1.00 | 32.32 | A |
| ATOM | 1887 | C | LYS | A | 254 | 35.741 | 35.749 | −9.912 | 1.00 | 24.07 | A |
| ATOM | 1888 | O | LYS | A | 254 | 35.532 | 34.893 | −9.048 | 1.00 | 23.89 | A |
| ATOM | 1889 | N | GLY | A | 255 | 35.888 | 37.040 | −9.628 | 1.00 | 23.13 | A |
| ATOM | 1890 | CA | GLY | A | 255 | 35.788 | 37.510 | −8.257 | 1.00 | 22.09 | A |
| ATOM | 1891 | C | GLY | A | 255 | 37.110 | 37.715 | −7.534 | 1.00 | 21.87 | A |
| ATOM | 1892 | O | GLY | A | 255 | 37.128 | 37.864 | −6.313 | 1.00 | 21.29 | A |
| ATOM | 1893 | N | ASP | A | 256 | 38.216 | 37.735 | −8.270 | 1.00 | 21.62 | A |
| ATOM | 1894 | CA | ASP | A | 256 | 39.525 | 37.919 | −7.648 | 1.00 | 21.86 | A |
| ATOM | 1895 | CB | ASP | A | 256 | 40.647 | 37.613 | −8.645 | 1.00 | 25.21 | A |
| ATOM | 1896 | CG | ASP | A | 256 | 40.638 | 36.171 | −9.112 | 1.00 | 28.23 | A |
| ATOM | 1897 | OD1 | ASP | A | 256 | 40.472 | 35.269 | −8.260 | 1.00 | 30.23 | A |
| ATOM | 1898 | OD2 | ASP | A | 256 | 40.808 | 35.941 | −10.329 | 1.00 | 30.53 | A |
| ATOM | 1899 | C | ASP | A | 256 | 39.741 | 39.319 | −7.074 | 1.00 | 20.26 | A |
| ATOM | 1900 | O | ASP | A | 256 | 40.663 | 39.532 | −6.291 | 1.00 | 19.39 | A |
| ATOM | 1901 | N | THR | A | 257 | 38.902 | 40.274 | −7.465 | 1.00 | 17.90 | A |
| ATOM | 1902 | CA | THR | A | 257 | 39.037 | 41.639 | −6.958 | 1.00 | 16.74 | A |
| ATOM | 1903 | CB | THR | A | 257 | 38.540 | 42.668 | −7.985 | 1.00 | 16.55 | A |
| ATOM | 1904 | OG1 | THR | A | 257 | 37.135 | 42.482 | −8.200 | 1.00 | 16.27 | A |
| ATOM | 1905 | OG2 | THR | A | 257 | 39.283 | 42.510 | −9.303 | 1.00 | 16.66 | A |
| ATOM | 1906 | C | THR | A | 257 | 38.233 | 41.848 | −5.675 | 1.00 | 16.22 | A |
| ATOM | 1907 | O | THR | A | 257 | 38.341 | 42.894 | −5.028 | 1.00 | 15.96 | A |
| ATOM | 1908 | N | ALA | A | 258 | 37.434 | 40.847 | −5.315 | 1.00 | 14.88 | A |
| ATOM | 1909 | CA | ALA | A | 258 | 36.578 | 40.912 | −4.137 | 1.00 | 14.24 | A |
| ATOM | 1910 | CB | ALA | A | 258 | 35.847 | 39.583 | −3.957 | 1.00 | 14.17 | A |
| ATOM | 1911 | C | ALA | A | 258 | 37.267 | 41.296 | −2.831 | 1.00 | 13.50 | A |
| ATOM | 1912 | O | ALA | A | 258 | 38.358 | 40.821 | −2.524 | 1.00 | 13.51 | A |
| ATOM | 1913 | N | LEU | A | 259 | 36.601 | 42.159 | −2.068 | 1.00 | 12.70 | A |
| ATOM | 1914 | CA | LEU | A | 259 | 37.092 | 42.610 | −0.768 | 1.00 | 12.24 | A |
| ATOM | 1915 | CB | LEU | A | 259 | 37.696 | 44.016 | −0.867 | 1.00 | 11.63 | A |
| ATOM | 1916 | CG | LEU | A | 259 | 38.380 | 44.520 | 0.408 | 1.00 | 11.76 | A |
| ATOM | 1917 | CD1 | LEU | A | 259 | 39.562 | 43.608 | 0.743 | 1.00 | 11.66 | A |
| ATOM | 1918 | CD2 | LEU | A | 259 | 38.849 | 45.963 | 0.220 | 1.00 | 12.00 | A |
| ATOM | 1919 | C | LEU | A | 259 | 35.901 | 42.634 | 0.186 | 1.00 | 11.86 | A |
| ATOM | 1920 | O | LEU | A | 259 | 34.875 | 43.245 | −0.111 | 1.00 | 11.05 | A |
| ATOM | 1921 | N | PHE | A | 260 | 36.032 | 41.959 | 1.324 | 1.00 | 11.65 | A |
| ATOM | 1922 | CA | PHE | A | 260 | 34.951 | 41.921 | 2.303 | 1.00 | 11.54 | A |
| ATOM | 1923 | CB | PHE | A | 260 | 35.345 | 41.055 | 3.501 | 1.00 | 11.54 | A |
| ATOM | 1924 | CG | PHE | A | 260 | 34.245 | 40.882 | 4.503 | 1.00 | 12.18 | A |
| ATOM | 1925 | CD1 | PHE | A | 260 | 33.242 | 39.934 | 4.301 | 1.00 | 12.98 | A |
| ATOM | 1926 | CD2 | PHE | A | 260 | 34.195 | 41.679 | 5.640 | 1.00 | 12.01 | A |
| ATOM | 1927 | CE1 | PHE | A | 260 | 32.209 | 39.786 | 5.218 | 1.00 | 12.59 | A |
| ATOM | 1928 | CE2 | PHE | A | 260 | 33.167 | 41.541 | 6.565 | 1.00 | 12.09 | A |
| ATOM | 1929 | CZ | PHE | A | 260 | 32.170 | 40.593 | 6.355 | 1.00 | 12.78 | A |
| ATOM | 1930 | C | PHE | A | 260 | 34.627 | 43.336 | 2.773 | 1.00 | 11.53 | A |
| ATOM | 1931 | O | PHE | A | 260 | 35.507 | 44.076 | 3.222 | 1.00 | 11.50 | A |
| ATOM | 1932 | N | THR | A | 261 | 33.356 | 43.707 | 2.680 | 1.00 | 11.54 | A |
| ATOM | 1933 | CA | THR | A | 261 | 32.931 | 45.044 | 3.061 | 1.00 | 11.72 | A |
| ATOM | 1934 | CB | THR | A | 261 | 32.464 | 45.834 | 1.822 | 1.00 | 12.10 | A |
| ATOM | 1935 | OG1 | THR | A | 261 | 33.510 | 45.841 | 0.842 | 1.00 | 12.08 | A |
| ATOM | 1936 | CG2 | THR | A | 261 | 32.114 | 47.273 | 2.195 | 1.00 | 12.27 | A |
| ATOM | 1937 | C | THR | A | 261 | 31.796 | 45.041 | 4.075 | 1.00 | 11.96 | A |
| ATOM | 1938 | O | THR | A | 261 | 30.841 | 44.275 | 3.954 | 1.00 | 11.91 | A |
| ATOM | 1939 | N | HIS | A | 262 | 31.909 | 45.918 | 5.065 | 1.00 | 11.38 | A |
| ATOM | 1940 | CA | HIS | A | 262 | 30.894 | 46.069 | 6.095 | 1.00 | 11.42 | A |
| ATOM | 1941 | CB | HIS | A | 262 | 31.522 | 45.971 | 7.485 | 1.00 | 12.15 | A |
| ATOM | 1942 | CG | HIS | A | 262 | 30.589 | 46.339 | 8.598 | 1.00 | 12.10 | A |
| ATOM | 1943 | CD2 | HIS | A | 262 | 30.188 | 47.547 | 9.065 | 1.00 | 12.09 | A |
| ATOM | 1944 | ND1 | HIS | A | 262 | 29.950 | 45.398 | 9.376 | 1.00 | 11.76 | A |
| ATOM | 1945 | CE1 | HIS | A | 262 | 29.199 | 46.010 | 10.277 | 1.00 | 12.27 | A |
| ATOM | 1946 | NE2 | HIS | A | 262 | 29.327 | 47.314 | 10.111 | 1.00 | 12.08 | A |
| ATOM | 1947 | C | HIS | A | 262 | 30.271 | 47.451 | 5.945 | 1.00 | 11.76 | A |
| ATOM | 1948 | O | HIS | A | 262 | 30.965 | 48.463 | 6.052 | 1.00 | 10.18 | A |
| ATOM | 1949 | N | MET | A | 263 | 28.968 | 47.491 | 5.689 | 1.00 | 11.17 | A |
| ATOM | 1950 | CA | MET | A | 263 | 28.267 | 48.759 | 5.561 | 1.00 | 12.18 | A |
| ATOM | 1951 | CB | MET | A | 263 | 27.257 | 48.715 | 4.410 | 1.00 | 11.99 | A |
| ATOM | 1952 | CG | MET | A | 263 | 26.484 | 50.024 | 4.207 | 1.00 | 12.03 | A |
| ATOM | 1953 | SD | MET | A | 263 | 25.055 | 49.848 | 3.084 | 1.00 | 13.44 | A |
| ATOM | 1954 | CE | MET | A | 263 | 25.884 | 49.600 | 1.504 | 1.00 | 12.15 | A |
| ATOM | 1955 | C | MET | A | 263 | 27.520 | 48.990 | 6.868 | 1.00 | 12.69 | A |
| ATOM | 1956 | O | MET | A | 263 | 26.764 | 48.122 | 7.314 | 1.00 | 11.94 | A |
| ATOM | 1957 | N | MET | A | 264 | 27.740 | 50.143 | 7.492 | 1.00 | 12.61 | A |
| ATOM | 1958 | CA | MET | A | 264 | 27.038 | 50.454 | 8.730 | 1.00 | 13.99 | A |
| ATOM | 1959 | CB | MET | A | 264 | 27.631 | 51.712 | 9.366 | 1.00 | 15.28 | A |
| ATOM | 1960 | CG | MET | A | 264 | 29.035 | 51.450 | 9.916 | 1.00 | 17.24 | A |
| ATOM | 1961 | SD | MET | A | 264 | 29.953 | 52.918 | 10.450 | 1.00 | 22.77 | A |
| ATOM | 1962 | CE | MET | A | 264 | 28.945 | 53.487 | 11.811 | 1.00 | 21.46 | A |
| ATOM | 1963 | C | MET | A | 264 | 25.573 | 50.613 | 8.326 | 1.00 | 14.16 | A |
| ATOM | 1964 | O | MET | A | 264 | 25.275 | 51.066 | 7.220 | 1.00 | 14.15 | A |
| ATOM | 1965 | N | PRO | A | 265 | 24.640 | 50.241 | 9.216 | 1.00 | 13.83 | A |
| ATOM | 1966 | CD | PRO | A | 265 | 24.882 | 49.677 | 10.559 | 1.00 | 13.64 | A |
| ATOM | 1967 | CA | PRO | A | 265 | 23.203 | 50.312 | 8.938 | 1.00 | 14.21 | A |
| ATOM | 1968 | CB | PRO | A | 265 | 22.661 | 49.173 | 9.787 | 1.00 | 14.20 | A |
| ATOM | 1969 | CG | PRO | A | 265 | 23.456 | 49.357 | 11.058 | 1.00 | 14.38 | A |
| ATOM | 1970 | C | PRO | A | 265 | 22.424 | 51.589 | 9.194 | 1.00 | 14.67 | A |
| ATOM | 1971 | O | PRO | A | 265 | 21.335 | 51.770 | 8.642 | 1.00 | 14.40 | A |
| ATOM | 1972 | N | PHE | A | 266 | 22.973 | 52.482 | 10.001 | 1.00 | 14.07 | A |
| ATOM | 1973 | CA | PHE | A | 266 | 22.229 | 53.671 | 10.359 | 1.00 | 14.30 | A |
| ATOM | 1974 | CB | PHE | A | 266 | 22.185 | 53.727 | 11.889 | 1.00 | 12.76 | A |
| ATOM | 1975 | CG | PHE | A | 266 | 21.650 | 52.451 | 12.517 | 1.00 | 13.20 | A |
| ATOM | 1976 | CD1 | PHE | A | 266 | 22.244 | 51.907 | 13.655 | 1.00 | 12.53 | A |
| ATOM | 1977 | CD2 | PHE | A | 266 | 20.555 | 51.792 | 11.957 | 1.00 | 12.83 | A |
| ATOM | 1978 | CE1 | PHE | A | 266 | 21.755 | 50.723 | 14.224 | 1.00 | 13.38 | A |
| ATOM | 1979 | CE2 | PHE | A | 266 | 20.055 | 50.610 | 12.515 | 1.00 | 12.47 | A |
| ATOM | 1980 | CZ | PHE | A | 266 | 20.655 | 50.073 | 13.651 | 1.00 | 12.89 | A |
| ATOM | 1981 | C | PHE | A | 266 | 22.600 | 55.025 | 9.743 | 1.00 | 14.18 | A |
| ATOM | 1982 | O | PHE | A | 266 | 23.519 | 55.139 | 8.930 | 1.00 | 14.66 | A |
| ATOM | 1983 | N | TYR | A | 267 | 21.841 | 56.038 | 10.142 | 1.00 | 14.48 | A |
| ATOM | 1984 | CA | TYR | A | 267 | 21.956 | 57.413 | 9.655 | 1.00 | 14.39 | A |
| ATOM | 1985 | CB | TYR | A | 267 | 20.829 | 58.232 | 10.294 | 1.00 | 15.80 | A |
| ATOM | 1986 | CG | TYR | A | 267 | 20.885 | 59.729 | 10.094 | 1.00 | 16.29 | A |
| ATOM | 1987 | CD1 | TYR | A | 267 | 20.538 | 60.310 | 8.877 | 1.00 | 17.30 | A |
| ATOM | 1988 | CE1 | TYR | A | 267 | 20.534 | 61.705 | 8.717 | 1.00 | 17.59 | A |
| ATOM | 1989 | CD2 | TYR | A | 267 | 21.239 | 60.571 | 11.149 | 1.00 | 16.80 | A |
| ATOM | 1990 | CE2 | TYR | A | 267 | 21.242 | 61.954 | 11.001 | 1.00 | 17.38 | A |
| ATOM | 1991 | CZ | TYR | A | 267 | 20.887 | 62.514 | 9.789 | 1.00 | 18.05 | A |
| ATOM | 1992 | OH | TYR | A | 267 | 20.876 | 63.883 | 9.659 | 1.00 | 18.70 | A |
| ATOM | 1993 | C | TYR | A | 267 | 23.293 | 58.120 | 9.861 | 1.00 | 14.81 | A |
| ATOM | 1994 | O | TYR | A | 267 | 23.699 | 58.943 | 9.035 | 1.00 | 13.98 | A |
| ATOM | 1995 | N | SER | A | 268 | 23.977 | 57.810 | 10.955 | 1.00 | 14.04 | A |
| ATOM | 1996 | CA | SER | A | 268 | 25.247 | 58.465 | 11.242 | 1.00 | 14.27 | A |
| ATOM | 1997 | CB | SER | A | 268 | 24.980 | 59.724 | 12.075 | 1.00 | 14.25 | A |
| ATOM | 1998 | OG | SER | A | 268 | 26.173 | 60.276 | 12.600 | 1.00 | 14.87 | A |
| ATOM | 1999 | C | SER | A | 268 | 26.230 | 57.559 | 11.972 | 1.00 | 13.84 | A |
| ATOM | 2000 | O | SER | A | 268 | 25.871 | 56.468 | 12.426 | 1.00 | 13.95 | A |
| ATOM | 2001 | N | TYR | A | 269 | 27.477 | 58.011 | 12.072 | 1.00 | 13.39 | A |
| ATOM | 2002 | CA | TYR | A | 269 | 28.503 | 57.255 | 12.773 | 1.00 | 12.49 | A |
| ATOM | 2003 | CB | TYR | A | 269 | 29.861 | 57.390 | 12.066 | 1.00 | 11.88 | A |
| ATOM | 2004 | CG | TYR | A | 269 | 30.310 | 58.820 | 11.840 | 1.00 | 11.45 | A |
| ATOM | 2005 | CD1 | TYR | A | 269 | 30.728 | 59.623 | 12.903 | 1.00 | 10.84 | A |
| ATOM | 2006 | CE1 | TYR | A | 269 | 31.110 | 60.956 | 12.698 | 1.00 | 12.04 | A |
| ATOM | 2007 | CD2 | TYR | A | 269 | 30.288 | 59.378 | 10.560 | 1.00 | 11.68 | A |
| ATOM | 2008 | CE2 | TYR | A | 269 | 30.666 | 60.704 | 10.343 | 1.00 | 12.15 | A |
| ATOM | 2009 | CZ | TYR | A | 269 | 31.070 | 61.487 | 11.411 | 1.00 | 11.80 | A |
| ATOM | 2010 | OH | TYR | A | 269 | 31.402 | 62.805 | 11.190 | 1.00 | 12.70 | A |
| ATOM | 2011 | C | TYR | A | 269 | 28.625 | 57.739 | 14.218 | 1.00 | 12.89 | A |
| ATOM | 2012 | O | TYR | A | 269 | 29.446 | 57.222 | 14.974 | 1.00 | 12.77 | A |
| ATOM | 2013 | N | ASP | A | 270 | 27.821 | 58.729 | 14.607 | 1.00 | 12.89 | A |
| ATOM | 2014 | CA | ASP | A | 270 | 27.897 | 59.218 | 15.984 | 1.00 | 12.80 | A |
| ATOM | 2015 | CB | ASP | A | 270 | 27.202 | 60.585 | 16.151 | 1.00 | 13.50 | A |
| ATOM | 2016 | CG | ASP | A | 270 | 25.709 | 60.549 | 15.862 | 1.00 | 14.42 | A |
| ATOM | 2017 | OD1 | ASP | A | 270 | 25.112 | 59.453 | 15.808 | 1.00 | 14.48 | A |
| ATOM | 2018 | OD2 | ASP | A | 270 | 25.129 | 61.649 | 15.708 | 1.00 | 15.21 | A |
| ATOM | 2019 | C | ASP | A | 270 | 27.312 | 58.179 | 16.936 | 1.00 | 13.07 | A |
| ATOM | 2020 | O | ASP | A | 270 | 26.657 | 57.232 | 16.504 | 1.00 | 12.66 | A |
| ATOM | 2021 | N | ILE | A | 271 | 27.556 | 58.343 | 18.230 | 1.00 | 12.62 | A |
| ATOM | 2022 | CA | ILE | A | 271 | 27.082 | 57.360 | 19.192 | 1.00 | 12.36 | A |
| ATOM | 2023 | CB | ILE | A | 271 | 27.586 | 57.716 | 20.610 | 1.00 | 11.96 | A |
| ATOM | 2024 | CG2 | ILE | A | 271 | 27.051 | 56.718 | 21.634 | 1.00 | 11.64 | A |
| ATOM | 2025 | CG1 | ILE | A | 271 | 29.120 | 57.666 | 20.615 | 1.00 | 12.08 | A |
| ATOM | 2026 | CD1 | ILE | A | 271 | 29.774 | 58.239 | 21.866 | 1.00 | 12.93 | A |
| ATOM | 2027 | C | ILE | A | 271 | 25.570 | 57.113 | 19.170 | 1.00 | 12.34 | A |
| ATOM | 2028 | O | ILE | A | 271 | 25.131 | 55.966 | 19.244 | 1.00 | 12.60 | A |
| ATOM | 2029 | N | PRO | A | 272 | 24.754 | 58.172 | 19.049 | 1.00 | 12.54 | A |
| ATOM | 2030 | CD | PRO | A | 272 | 25.057 | 59.612 | 19.135 | 1.00 | 12.73 | A |
| ATOM | 2031 | CA | PRO | A | 272 | 23.305 | 57.943 | 19.022 | 1.00 | 12.67 | A |
| ATOM | 2032 | CB | PRO | A | 272 | 22.737 | 59.352 | 18.875 | 1.00 | 12.85 | A |
| ATOM | 2033 | CG | PRO | A | 272 | 23.745 | 60.190 | 19.610 | 1.00 | 13.34 | A |
| ATOM | 2034 | C | PRO | A | 272 | 22.856 | 57.017 | 17.881 | 1.00 | 13.24 | A |
| ATOM | 2035 | O | PRO | A | 272 | 21.801 | 56.394 | 17.966 | 1.00 | 13.14 | A |
| ATOM | 2036 | N | HIS | A | 273 | 23.661 | 56.918 | 16.825 | 1.00 | 12.69 | A |
| ATOM | 2037 | CA | HIS | A | 273 | 23.313 | 56.077 | 15.678 | 1.00 | 13.21 | A |
| ATOM | 2038 | CB | HIS | A | 273 | 23.273 | 56.938 | 14.412 | 1.00 | 13.31 | A |
| ATOM | 2039 | CG | HIS | A | 273 | 22.261 | 58.040 | 14.473 | 1.00 | 14.15 | A |
| ATOM | 2040 | CD2 | HIS | A | 273 | 22.363 | 59.319 | 14.904 | 1.00 | 13.91 | A |
| ATOM | 2041 | ND1 | HIS | A | 273 | 20.940 | 57.859 | 14.121 | 1.00 | 15.41 | A |
| ATOM | 2042 | CE1 | HIS | A | 273 | 20.273 | 58.979 | 14.333 | 1.00 | 13.45 | A |
| ATOM | 2043 | NE2 | HIS | A | 273 | 21.112 | 59.881 | 14.809 | 1.00 | 16.34 | A |
| ATOM | 2044 | C | HIS | A | 273 | 24.233 | 54.876 | 15.457 | 1.00 | 13.37 | A |
| ATOM | 2045 | O | HIS | A | 273 | 24.334 | 54.360 | 14.339 | 1.00 | 12.85 | A |
| ATOM | 2046 | N | THR | A | 274 | 24.895 | 54.421 | 16.518 | 1.00 | 13.98 | A |
| ATOM | 2047 | CA | THR | A | 274 | 25.788 | 53.276 | 16.395 | 1.00 | 13.68 | A |
| ATOM | 2048 | CB | THR | A | 274 | 27.274 | 53.726 | 16.415 | 1.00 | 13.90 | A |
| ATOM | 2049 | OG1 | THR | A | 274 | 27.480 | 54.682 | 17.459 | 1.00 | 13.47 | A |
| ATOM | 2050 | CG2 | THR | A | 274 | 27.656 | 54.346 | 15.079 | 1.00 | 12.79 | A |
| ATOM | 2051 | C | THR | A | 274 | 25.586 | 52.153 | 17.420 | 1.00 | 14.02 | A |
| ATOM | 2052 | O | THR | A | 274 | 26.143 | 51.069 | 17.257 | 1.00 | 14.90 | A |
| ATOM | 2053 | N | CYS | A | 275 | 24.795 | 52.390 | 18.466 | 1.00 | 14.38 | A |
| ATOM | 2054 | CA | CYS | A | 275 | 24.569 | 51.344 | 19.471 | 1.00 | 14.59 | A |
| ATOM | 2055 | C | CYS | A | 275 | 23.380 | 50.457 | 19.110 | 1.00 | 14.87 | A |
| ATOM | 2056 | O | CYS | A | 275 | 23.303 | 49.298 | 19.525 | 1.00 | 14.62 | A |
| ATOM | 2057 | CB | CYS | A | 275 | 24.331 | 51.965 | 20.851 | 1.00 | 15.94 | A |
| ATOM | 2058 | SG | CYS | A | 275 | 22.592 | 52.094 | 21.403 | 1.00 | 16.28 | A |
| ATOM | 2059 | N | GLY | A | 276 | 22.456 | 51.017 | 18.337 | 1.00 | 14.95 | A |
| ATOM | 2060 | CA | GLY | A | 276 | 21.266 | 50.290 | 17.940 | 1.00 | 15.51 | A |
| ATOM | 2061 | C | GLY | A | 276 | 20.345 | 51.178 | 17.124 | 1.00 | 15.57 | A |
| ATOM | 2062 | O | GLY | A | 276 | 20.714 | 52.312 | 16.811 | 1.00 | 15.76 | A |
| ATOM | 2063 | N | PRO | A | 277 | 19.131 | 50.704 | 16.783 | 1.00 | 14.99 | A |
| ATOM | 2064 | CD | PRO | A | 277 | 18.615 | 49.379 | 17.174 | 1.00 | 15.17 | A |
| ATOM | 2065 | CA | PRO | A | 277 | 18.129 | 51.430 | 15.993 | 1.00 | 15.46 | A |
| ATOM | 2066 | CB | PRO | A | 277 | 17.105 | 50.343 | 15.667 | 1.00 | 15.74 | A |
| ATOM | 2067 | CG | PRO | A | 277 | 17.128 | 49.514 | 16.907 | 1.00 | 14.95 | A |
| ATOM | 2068 | C | PRO | A | 277 | 17.462 | 52.650 | 16.626 | 1.00 | 15.44 | A |
| ATOM | 2069 | O | PRO | A | 277 | 16.880 | 53.468 | 15.915 | 1.00 | 15.14 | A |
| ATOM | 2070 | N | ASP | A | 278 | 17.536 | 52.777 | 17.948 | 1.00 | 15.72 | A |
| ATOM | 2071 | CA | ASP | A | 278 | 16.887 | 53.899 | 18.628 | 1.00 | 16.37 | A |
| ATOM | 2072 | CB | ASP | A | 278 | 16.015 | 53.382 | 19.778 | 1.00 | 16.45 | A |
| ATOM | 2073 | CG | ASP | A | 278 | 15.089 | 54.449 | 20.333 | 1.00 | 17.33 | A |
| ATOM | 2074 | OD1 | ASP | A | 278 | 15.253 | 55.635 | 19.972 | 1.00 | 17.28 | A |
| ATOM | 2075 | OD2 | ASP | A | 278 | 14.201 | 54.102 | 21.134 | 1.00 | 16.81 | A |
| ATOM | 2076 | C | ASP | A | 278 | 17.865 | 54.934 | 19.174 | 1.00 | 16.20 | A |
| ATOM | 2077 | O | ASP | A | 278 | 18.482 | 54.729 | 20.219 | 1.00 | 15.93 | A |
| ATOM | 2078 | N | PRO | A | 279 | 18.003 | 56.073 | 18.480 | 1.00 | 16.64 | A |
| ATOM | 2079 | CD | PRO | A | 279 | 17.320 | 56.464 | 17.234 | 1.00 | 16.82 | A |
| ATOM | 2080 | CA | PRO | A | 279 | 18.921 | 57.126 | 18.928 | 1.00 | 17.09 | A |
| ATOM | 2081 | CB | PRO | A | 279 | 18.850 | 58.152 | 17.793 | 1.00 | 16.72 | A |
| ATOM | 2082 | CG | PRO | A | 279 | 17.464 | 57.968 | 17.253 | 1.00 | 17.23 | A |
| ATOM | 2083 | C | PRO | A | 279 | 18.571 | 57.723 | 20.291 | 1.00 | 17.34 | A |
| ATOM | 2084 | O | PRO | A | 279 | 19.447 | 58.221 | 20.996 | 1.00 | 17.37 | A |
| ATOM | 2085 | N | LYS | A | 280 | 17.296 | 57.676 | 20.667 | 1.00 | 17.71 | A |
| ATOM | 2086 | CA | LYS | A | 280 | 16.887 | 58.217 | 21.960 | 1.00 | 18.13 | A |
| ATOM | 2087 | CB | LYS | A | 280 | 15.363 | 58.214 | 22.097 | 1.00 | 19.17 | A |
| ATOM | 2088 | CG | LYS | A | 280 | 14.871 | 58.892 | 23.370 | 1.00 | 21.45 | A |
| ATOM | 2089 | CD | LYS | A | 280 | 13.358 | 58.837 | 23.491 | 1.00 | 23.26 | A |
| ATOM | 2090 | CE | LYS | A | 280 | 12.876 | 57.413 | 23.710 | 1.00 | 26.06 | A |
| ATOM | 2091 | NZ | LYS | A | 280 | 13.446 | 56.812 | 24.960 | 1.00 | 28.16 | A |
| ATOM | 2092 | C | LYS | A | 280 | 17.500 | 57.389 | 23.081 | 1.00 | 17.62 | A |
| ATOM | 2093 | O | LYS | A | 280 | 17.784 | 57.902 | 24.165 | 1.00 | 18.50 | A |
| ATOM | 2094 | N | VAL | A | 281 | 17.702 | 56.102 | 22.819 | 1.00 | 17.07 | A |
| ATOM | 2095 | CA | VAL | A | 281 | 18.299 | 55.215 | 23.810 | 1.00 | 16.41 | A |
| ATOM | 2096 | CB | VAL | A | 281 | 17.845 | 53.748 | 23.599 | 1.00 | 17.36 | A |
| ATOM | 2097 | CG1 | VAL | A | 281 | 18.577 | 52.827 | 24.568 | 1.00 | 16.56 | A |
| ATOM | 2098 | CG2 | VAL | A | 281 | 16.334 | 53.631 | 23.793 | 1.00 | 16.45 | A |
| ATOM | 2099 | C | VAL | A | 281 | 19.824 | 55.281 | 23.705 | 1.00 | 16.33 | A |
| ATOM | 2100 | O | VAL | A | 281 | 20.522 | 55.440 | 24.705 | 1.00 | 15.54 | A |
| ATOM | 2101 | N | CYS | A | 282 | 20.337 | 55.163 | 22.484 | 1.00 | 15.75 | A |
| ATOM | 2102 | CA | CYS | A | 282 | 21.780 | 55.198 | 22.266 | 1.00 | 16.11 | A |
| ATOM | 2103 | C | CYS | A | 282 | 22.426 | 56.483 | 22.773 | 1.00 | 16.13 | A |
| ATOM | 2104 | O | CYS | A | 282 | 23.551 | 56.463 | 23.282 | 1.00 | 16.09 | A |
| ATOM | 2105 | CB | CYS | A | 282 | 22.096 | 55.029 | 20.780 | 1.00 | 15.93 | A |
| ATOM | 2106 | SG | CYS | A | 282 | 21.730 | 53.383 | 20.093 | 1.00 | 16.46 | A |
| ATOM | 2107 | N | CYS | A | 283 | 21.720 | 57.600 | 22.640 | 1.00 | 15.64 | A |
| ATOM | 2108 | CA | CYS | A | 283 | 22.271 | 58.870 | 23.088 | 1.00 | 16.03 | A |
| ATOM | 2109 | C | CYS | A | 283 | 22.575 | 58.830 | 24.582 | 1.00 | 16.35 | A |
| ATOM | 2110 | O | CYS | A | 283 | 23.480 | 59.514 | 25.061 | 1.00 | 16.19 | A |
| ATOM | 2111 | CB | CYS | A | 283 | 21.309 | 60.023 | 22.785 | 1.00 | 17.15 | A |
| ATOM | 2112 | SG | CYS | A | 283 | 22.169 | 61.628 | 22.845 | 1.00 | 17.80 | A |
| ATOM | 2113 | N | GLN | A | 284 | 21.820 | 58.019 | 25.315 | 1.00 | 16.07 | A |
| ATOM | 2114 | CA | GLN | A | 284 | 22.013 | 57.898 | 26.754 | 1.00 | 16.46 | A |
| ATOM | 2115 | CB | GLN | A | 284 | 20.823 | 57.169 | 27.382 | 1.00 | 16.54 | A |
| ATOM | 2116 | CG | GLN | A | 284 | 19.513 | 57.918 | 27.223 | 1.00 | 17.45 | A |
| ATOM | 2117 | CD | GLN | A | 284 | 18.334 | 57.129 | 27.737 | 1.00 | 17.46 | A |
| ATOM | 2118 | OE1 | GLN | A | 284 | 18.291 | 56.743 | 28.907 | 1.00 | 18.95 | A |
| ATOM | 2119 | NE2 | GLN | A | 284 | 17.367 | 56.880 | 26.865 | 1.00 | 18.72 | A |
| ATOM | 2120 | C | GLN | A | 284 | 23.300 | 57.168 | 27.095 | 1.00 | 16.25 | A |
| ATOM | 2121 | O | GLN | A | 284 | 23.680 | 57.077 | 28.262 | 1.00 | 16.37 | A |
| ATOM | 2122 | N | PHE | A | 285 | 23.978 | 56.654 | 26.075 | 1.00 | 15.74 | A |
| ATOM | 2123 | CA | PHE | A | 285 | 25.216 | 55.938 | 26.309 | 1.00 | 15.95 | A |
| ATOM | 2124 | CB | PHE | A | 285 | 25.060 | 54.495 | 25.821 | 1.00 | 16.02 | A |
| ATOM | 2125 | CG | PHE | A | 285 | 24.080 | 53.705 | 26.652 | 1.00 | 16.47 | A |
| ATOM | 2126 | CD1 | PHE | A | 285 | 24.487 | 53.090 | 27.832 | 1.00 | 16.88 | A |
| ATOM | 2127 | CD2 | PHE | A | 285 | 22.732 | 53.660 | 26.306 | 1.00 | 17.20 | A |
| ATOM | 2128 | CE1 | PHE | A | 285 | 23.566 | 52.444 | 28.664 | 1.00 | 17.28 | A |
| ATOM | 2129 | CE2 | PHE | A | 285 | 21.799 | 53.017 | 27.129 | 1.00 | 18.31 | A |
| ATOM | 2130 | CZ | PHE | A | 285 | 22.220 | 52.409 | 28.312 | 1.00 | 18.03 | A |
| ATOM | 2131 | C | PHE | A | 285 | 26.439 | 56.637 | 25.730 | 1.00 | 15.58 | A |
| ATOM | 2132 | O | PHE | A | 285 | 27.481 | 56.025 | 25.500 | 1.00 | 15.28 | A |
| ATOM | 2133 | N | ASP | A | 286 | 26.282 | 57.938 | 25.496 | 1.00 | 15.60 | A |
| ATOM | 2134 | CA | ASP | A | 286 | 27.365 | 58.802 | 25.039 | 1.00 | 15.26 | A |
| ATOM | 2135 | CB | ASP | A | 286 | 26.926 | 59.691 | 23.875 | 1.00 | 15.15 | A |
| ATOM | 2136 | CG | ASP | A | 286 | 28.043 | 60.603 | 23.389 | 1.00 | 15.24 | A |
| ATOM | 2137 | OD1 | ASP | A | 286 | 29.086 | 60.683 | 24.074 | 1.00 | 15.09 | A |
| ATOM | 2138 | OD2 | ASP | A | 286 | 27.876 | 61.247 | 22.331 | 1.00 | 14.65 | A |
| ATOM | 2139 | C | ASP | A | 286 | 27.543 | 59.644 | 26.296 | 1.00 | 15.45 | A |
| ATOM | 2140 | O | ASP | A | 286 | 26.896 | 60.677 | 26.468 | 1.00 | 15.12 | A |
| ATOM | 2141 | N | PHE | A | 287 | 28.414 | 59.189 | 27.186 | 1.00 | 15.64 | A |
| ATOM | 2142 | CA | PHE | A | 287 | 28.606 | 59.873 | 28.449 | 1.00 | 16.17 | A |
| ATOM | 2143 | CB | PHE | A | 287 | 29.340 | 58.939 | 29.411 | 1.00 | 15.78 | A |
| ATOM | 2144 | CG | PHE | A | 287 | 28.596 | 57.652 | 29.661 | 1.00 | 15.58 | A |
| ATOM | 2145 | CD1 | PHE | A | 287 | 28.811 | 56.538 | 28.856 | 1.00 | 14.97 | A |
| ATOM | 2146 | CD2 | PHE | A | 287 | 27.614 | 57.584 | 30.646 | 1.00 | 15.57 | A |
| ATOM | 2147 | CE1 | PHE | A | 287 | 28.056 | 55.378 | 29.025 | 1.00 | 15.24 | A |
| ATOM | 2148 | CE2 | PHE | A | 287 | 26.852 | 56.428 | 30.821 | 1.00 | 15.22 | A |
| ATOM | 2149 | CZ | PHE | A | 287 | 27.074 | 55.323 | 30.008 | 1.00 | 15.06 | A |
| ATOM | 2150 | C | PHE | A | 287 | 29.236 | 61.258 | 28.424 | 1.00 | 17.09 | A |
| ATOM | 2151 | O | PHE | A | 287 | 29.471 | 61.850 | 29.471 | 1.00 | 16.59 | A |
| ATOM | 2152 | N | LYS | A | 288 | 29.493 | 61.788 | 27.232 | 1.00 | 17.81 | A |
| ATOM | 2153 | CA | LYS | A | 288 | 30.053 | 63.131 | 27.139 | 1.00 | 17.75 | A |
| ATOM | 2154 | CB | LYS | A | 288 | 30.972 | 63.261 | 25.912 | 1.00 | 17.97 | A |
| ATOM | 2155 | CG | LYS | A | 288 | 31.716 | 64.598 | 25.846 | 1.00 | 17.53 | A |
| ATOM | 2156 | CD | LYS | A | 288 | 32.660 | 64.693 | 24.649 | 1.00 | 16.44 | A |
| ATOM | 2157 | CE | LYS | A | 288 | 33.438 | 66.003 | 24.686 | 1.00 | 17.49 | A |
| ATOM | 2158 | NZ | LYS | A | 288 | 34.386 | 66.162 | 23.535 | 1.00 | 17.95 | A |
| ATOM | 2159 | C | LYS | A | 288 | 28.900 | 64.132 | 27.025 | 1.00 | 18.16 | A |
| ATOM | 2160 | O | LYS | A | 288 | 29.121 | 65.337 | 26.953 | 1.00 | 17.87 | A |
| ATOM | 2161 | N | ARG | A | 289 | 27.667 | 63.634 | 27.027 | 1.00 | 19.07 | A |
| ATOM | 2162 | CA | ARG | A | 289 | 26.507 | 64.513 | 26.891 | 1.00 | 20.41 | A |
| ATOM | 2163 | CB | ARG | A | 289 | 25.549 | 63.961 | 25.830 | 1.00 | 19.03 | A |
| ATOM | 2164 | CG | ARG | A | 289 | 26.173 | 63.698 | 24.468 | 1.00 | 17.69 | A |
| ATOM | 2165 | CD | ARG | A | 289 | 25.092 | 63.465 | 23.431 | 1.00 | 17.66 | A |
| ATOM | 2166 | NE | ARG | A | 289 | 25.623 | 63.059 | 22.132 | 1.00 | 16.20 | A |
| ATOM | 2167 | CZ | ARG | A | 289 | 25.144 | 63.492 | 20.970 | 1.00 | 16.63 | A |
| ATOM | 2168 | NH1 | ARG | A | 289 | 24.132 | 64.353 | 20.949 | 1.00 | 15.95 | A |
| ATOM | 2169 | NH2 | ARG | A | 289 | 25.660 | 63.052 | 19.831 | 1.00 | 15.30 | A |
| ATOM | 2170 | C | ARG | A | 289 | 25.711 | 64.769 | 28.171 | 1.00 | 22.16 | A |
| ATOM | 2171 | O | ARG | A | 289 | 24.487 | 64.884 | 28.118 | 1.00 | 22.14 | A |
| ATOM | 2172 | N | MET | A | 290 | 26.381 | 64.881 | 29.312 | 1.00 | 24.35 | A |
| ATOM | 2173 | CA | MET | A | 290 | 25.653 | 65.113 | 30.556 | 1.00 | 26.07 | A |
| ATOM | 2174 | CB | MET | A | 290 | 26.232 | 64.241 | 31.677 | 1.00 | 27.03 | A |
| ATOM | 2175 | CG | MET | A | 290 | 26.008 | 62.749 | 31.441 | 1.00 | 27.88 | A |
| ATOM | 2176 | SD | MET | A | 290 | 26.517 | 61.649 | 32.787 | 1.00 | 29.89 | A |
| ATOM | 2177 | CE | MET | A | 290 | 28.257 | 61.423 | 32.409 | 1.00 | 28.24 | A |
| ATOM | 2178 | C | MET | A | 290 | 25.566 | 66.577 | 30.999 | 1.00 | 26.78 | A |
| ATOM | 2179 | O | MET | A | 290 | 24.848 | 66.894 | 31.953 | 1.00 | 28.04 | A |
| ATOM | 2180 | N | GLY | A | 291 | 26.280 | 67.469 | 30.311 | 1.00 | 26.38 | A |
| ATOM | 2181 | CA | GLY | A | 291 | 26.213 | 68.879 | 30.666 | 1.00 | 25.03 | A |
| ATOM | 2182 | C | GLY | A | 291 | 27.500 | 69.688 | 30.697 | 1.00 | 24.62 | A |
| ATOM | 2183 | O | GLY | A | 291 | 27.636 | 70.675 | 29.967 | 1.00 | 23.97 | A |
| ATOM | 2184 | N | SER | A | 292 | 28.442 | 69.282 | 31.546 | 1.00 | 22.93 | A |
| ATOM | 2185 | CA | SER | A | 292 | 29.711 | 69.989 | 31.691 | 1.00 | 21.82 | A |
| ATOM | 2186 | CB | SER | A | 292 | 30.584 | 69.289 | 32.734 | 1.00 | 22.87 | A |
| ATOM | 2187 | OG | SER | A | 292 | 30.945 | 67.989 | 32.304 | 1.00 | 23.87 | A |
| ATOM | 2188 | C | SER | A | 292 | 30.512 | 70.161 | 30.399 | 1.00 | 20.97 | A |
| ATOM | 2189 | O | SER | A | 292 | 31.381 | 71.032 | 30.321 | 1.00 | 19.65 | A |
| ATOM | 2190 | N | PHE | A | 293 | 30.231 | 69.333 | 29.394 | 1.00 | 19.55 | A |
| ATOM | 2191 | CA | PHE | A | 293 | 30.942 | 69.418 | 28.119 | 1.00 | 19.27 | A |
| ATOM | 2192 | CB | PHE | A | 293 | 31.173 | 68.021 | 27.530 | 1.00 | 19.08 | A |
| ATOM | 2193 | CG | PHE | A | 293 | 32.119 | 67.173 | 28.329 | 1.00 | 18.85 | A |
| ATOM | 2194 | CD1 | PHE | A | 293 | 31.641 | 66.288 | 29.286 | 1.00 | 19.32 | A |
| ATOM | 2195 | CD2 | PHE | A | 293 | 33.491 | 67.266 | 28.127 | 1.00 | 18.76 | A |
| ATOM | 2196 | CE1 | PHE | A | 293 | 32.517 | 65.501 | 30.032 | 1.00 | 19.71 | A |
| ATOM | 2197 | CE2 | PHE | A | 293 | 34.379 | 66.487 | 28.866 | 1.00 | 19.38 | A |
| ATOM | 2198 | CZ | PHE | A | 293 | 33.892 | 65.601 | 29.821 | 1.00 | 19.67 | A |
| ATOM | 2199 | C | PHE | A | 293 | 30.204 | 70.271 | 27.091 | 1.00 | 19.29 | A |
| ATOM | 2200 | O | PHE | A | 293 | 30.661 | 70.420 | 25.954 | 1.00 | 17.76 | A |
| ATOM | 2201 | N | GLY | A | 294 | 29.063 | 70.825 | 27.489 | 1.00 | 19.35 | A |
| ATOM | 2202 | CA | GLY | A | 294 | 28.295 | 71.652 | 26.576 | 1.00 | 19.35 | A |
| ATOM | 2203 | C | GLY | A | 294 | 27.538 | 70.859 | 25.525 | 1.00 | 19.82 | A |
| ATOM | 2204 | O | GLY | A | 294 | 27.101 | 71.414 | 24.515 | 1.00 | 20.44 | A |
| ATOM | 2205 | N | LEU | A | 295 | 27.386 | 69.558 | 25.757 | 1.00 | 19.26 | A |
| ATOM | 2206 | CA | LEU | A | 295 | 26.670 | 68.694 | 24.826 | 1.00 | 18.91 | A |
| ATOM | 2207 | CB | LEU | A | 295 | 27.576 | 67.555 | 24.344 | 1.00 | 19.27 | A |
| ATOM | 2208 | CG | LEU | A | 295 | 28.849 | 67.928 | 23.578 | 1.00 | 19.09 | A |
| ATOM | 2209 | CD1 | LEU | A | 295 | 29.678 | 66.677 | 23.321 | 1.00 | 19.10 | A |
| ATOM | 2210 | CD2 | LEU | A | 295 | 28.479 | 68.607 | 22.261 | 1.00 | 19.06 | A |
| ATOM | 2211 | C | LEU | A | 295 | 25.448 | 68.108 | 25.525 | 1.00 | 19.05 | A |
| ATOM | 2212 | O | LEU | A | 295 | 25.417 | 68.003 | 26.749 | 1.00 | 19.27 | A |
| ATOM | 2213 | N | SER | A | 296 | 24.445 | 67.731 | 24.742 | 1.00 | 19.20 | A |
| ATOM | 2214 | CA | SER | A | 296 | 23.229 | 67.149 | 25.294 | 1.00 | 19.84 | A |
| ATOM | 2215 | CB | SER | A | 296 | 22.259 | 68.252 | 25.732 | 1.00 | 19.56 | A |
| ATOM | 2216 | OG | SER | A | 296 | 21.869 | 69.062 | 24.637 | 1.00 | 19.91 | A |
| ATOM | 2217 | C | SER | A | 296 | 22.564 | 66.263 | 24.253 | 1.00 | 19.90 | A |
| ATOM | 2218 | O | SER | A | 296 | 23.019 | 66.184 | 23.110 | 1.00 | 19.47 | A |
| ATOM | 2219 | N | CYS | A | 297 | 21.488 | 65.597 | 24.661 | 1.00 | 20.34 | A |
| ATOM | 2220 | CA | CYS | A | 297 | 20.741 | 64.713 | 23.776 | 1.00 | 20.55 | A |
| ATOM | 2221 | C | CYS | A | 297 | 19.469 | 65.383 | 23.268 | 1.00 | 21.46 | A |
| ATOM | 2222 | O | CYS | A | 297 | 18.579 | 65.721 | 24.047 | 1.00 | 22.22 | A |
| ATOM | 2223 | CB | CYS | A | 297 | 20.394 | 63.415 | 24.509 | 1.00 | 20.17 | A |
| ATOM | 2224 | SG | CYS | A | 297 | 21.832 | 62.324 | 24.724 | 1.00 | 19.68 | A |
| ATOM | 2225 | N | PRO | A | 298 | 19.368 | 65.583 | 21.946 | 1.00 | 21.80 | A |
| ATOM | 2226 | CD | PRO | A | 298 | 20.350 | 65.215 | 20.909 | 1.00 | 21.82 | A |
| ATOM | 2227 | CA | PRO | A | 298 | 18.192 | 66.219 | 21.346 | 1.00 | 22.23 | A |
| ATOM | 2228 | CB | PRO | A | 298 | 18.583 | 66.349 | 19.875 | 1.00 | 22.37 | A |
| ATOM | 2229 | CG | PRO | A | 298 | 19.498 | 65.187 | 19.664 | 1.00 | 22.37 | A |
| ATOM | 2230 | C | PRO | A | 298 | 16.892 | 65.441 | 21.541 | 1.00 | 22.49 | A |
| ATOM | 2231 | O | PRO | A | 298 | 15.805 | 66.004 | 21.411 | 1.00 | 22.25 | A |
| ATOM | 2232 | N | TRP | A | 299 | 17.006 | 64.153 | 21.856 | 1.00 | 22.24 | A |
| ATOM | 2233 | CA | TRP | A | 299 | 15.829 | 63.316 | 22.069 | 1.00 | 22.85 | A |
| ATOM | 2234 | CB | TRP | A | 299 | 16.174 | 61.845 | 21.781 | 1.00 | 21.59 | A |
| ATOM | 2235 | CG | TRP | A | 299 | 16.531 | 61.619 | 20.329 | 1.00 | 20.98 | A |
| ATOM | 2236 | CD2 | TRP | A | 299 | 17.846 | 61.653 | 19.747 | 1.00 | 20.85 | A |
| ATOM | 2237 | CE2 | TRP | A | 299 | 17.691 | 61.495 | 18.351 | 1.00 | 20.88 | A |
| ATOM | 2238 | CE3 | TRP | A | 299 | 19.137 | 61.808 | 20.270 | 1.00 | 20.11 | A |
| ATOM | 2239 | CD1 | TRP | A | 299 | 15.660 | 61.438 | 19.288 | 1.00 | 20.52 | A |
| ATOM | 2240 | NE1 | TRP | A | 299 | 16.350 | 61.364 | 18.098 | 1.00 | 21.16 | A |
| ATOM | 2241 | CZ2 | TRP | A | 299 | 18.781 | 61.488 | 17.470 | 1.00 | 21.31 | A |
| ATOM | 2242 | CZ3 | TRP | A | 299 | 20.221 | 61.801 | 19.394 | 1.00 | 21.02 | A |
| ATOM | 2243 | CH2 | TRP | A | 299 | 20.033 | 61.642 | 18.008 | 1.00 | 20.63 | A |
| ATOM | 2244 | C | TRP | A | 299 | 15.264 | 63.495 | 23.483 | 1.00 | 23.67 | A |
| ATOM | 2245 | O | TRP | A | 299 | 14.364 | 62.767 | 23.903 | 1.00 | 23.41 | A |
| ATOM | 2246 | N | LYS | A | 300 | 15.814 | 64.470 | 24.208 | 1.00 | 24.99 | A |
| ATOM | 2247 | CA | LYS | A | 300 | 15.363 | 64.832 | 25.558 | 1.00 | 25.96 | A |
| ATOM | 2248 | CB | LYS | A | 300 | 13.856 | 65.087 | 25.543 | 1.00 | 27.34 | A |
| ATOM | 2249 | CG | LYS | A | 300 | 13.416 | 66.108 | 24.516 | 1.00 | 29.33 | A |
| ATOM | 2250 | CD | LYS | A | 300 | 11.914 | 66.327 | 24.577 | 1.00 | 31.29 | A |
| ATOM | 2251 | CE | LYS | A | 300 | 11.454 | 67.255 | 23.468 | 1.00 | 32.58 | A |
| ATOM | 2252 | NZ | LYS | A | 300 | 12.209 | 68.536 | 23.490 | 1.00 | 34.20 | A |
| ATOM | 2253 | C | LYS | A | 300 | 15.686 | 63.912 | 26.735 | 1.00 | 25.72 | A |
| ATOM | 2254 | O | LYS | A | 300 | 15.271 | 64.187 | 27.859 | 1.00 | 25.71 | A |
| ATOM | 2255 | N | VAL | A | 301 | 16.401 | 62.821 | 26.493 | 1.00 | 25.17 | A |
| ATOM | 2256 | CA | VAL | A | 301 | 16.765 | 61.917 | 27.576 | 1.00 | 24.82 | A |
| ATOM | 2257 | CB | VAL | A | 301 | 16.254 | 60.484 | 27.323 | 1.00 | 25.15 | A |
| ATOM | 2258 | CG1 | VAL | A | 301 | 16.543 | 59.615 | 28.533 | 1.00 | 24.99 | A |
| ATOM | 2259 | CG2 | VAL | A | 301 | 14.760 | 60.506 | 27.034 | 1.00 | 25.25 | A |
| ATOM | 2260 | C | VAL | A | 301 | 18.287 | 61.906 | 27.672 | 1.00 | 24.84 | A |
| ATOM | 2261 | O | VAL | A | 301 | 18.973 | 61.397 | 26.785 | 1.00 | 24.60 | A |
| ATOM | 2262 | N | PRO | A | 302 | 18.834 | 62.476 | 23.756 | 1.00 | 24.68 | A |
| ATOM | 2263 | CD | PRO | A | 302 | 18.109 | 63.142 | 29.855 | 1.00 | 24.61 | A |
| ATOM | 2264 | CA | PRO | A | 302 | 20.280 | 62.547 | 28.976 | 1.00 | 24.22 | A |
| ATOM | 2265 | CB | PRO | A | 302 | 20.403 | 63.655 | 30.009 | 1.00 | 24.42 | A |
| ATOM | 2266 | CG | PRO | A | 302 | 19.207 | 63.391 | 30.875 | 1.00 | 24.71 | A |
| ATOM | 2267 | C | PRO | A | 302 | 20.904 | 61.253 | 29.470 | 1.00 | 23.88 | A |
| ATOM | 2268 | O | PRO | A | 302 | 20.224 | 60.390 | 30.019 | 1.00 | 23.34 | A |
| ATOM | 2269 | N | PRO | A | 303 | 22.219 | 61.096 | 29.264 | 1.00 | 23.79 | A |
| ATOM | 2270 | CD | PRO | A | 303 | 23.170 | 61.936 | 28.513 | 1.00 | 23.70 | A |
| ATOM | 2271 | CA | PRO | A | 303 | 22.853 | 59.867 | 29.740 | 1.00 | 23.66 | A |
| ATOM | 2272 | CB | PRO | A | 303 | 24.212 | 59.884 | 29.043 | 1.00 | 23.89 | A |
| ATOM | 2273 | CG | PRO | A | 303 | 24.507 | 61.346 | 28.915 | 1.00 | 24.48 | A |
| ATOM | 2274 | C | PRO | A | 303 | 22.968 | 59.964 | 31.258 | 1.00 | 24.01 | A |
| ATOM | 2275 | O | PRO | A | 303 | 23.011 | 61.065 | 31.815 | 1.00 | 23.16 | A |
| ATOM | 2276 | N | ARG | A | 304 | 22.997 | 58.820 | 31.927 | 1.00 | 24.25 | A |
| ATOM | 2277 | CA | ARG | A | 304 | 23.115 | 58.801 | 33.378 | 1.00 | 25.17 | A |
| ATOM | 2278 | CB | ARG | A | 304 | 21.833 | 58.251 | 34.013 | 1.00 | 26.88 | A |
| ATOM | 2279 | CG | ARG | A | 304 | 20.700 | 59.270 | 34.090 | 1.00 | 29.46 | A |
| ATOM | 2280 | CD | ARG | A | 304 | 19.390 | 58.630 | 34.533 | 1.00 | 32.28 | A |
| ATOM | 2281 | NE | ARG | A | 304 | 18.811 | 57.784 | 33.491 | 1.00 | 34.83 | A |
| ATOM | 2282 | CZ | ARG | A | 304 | 18.400 | 58.229 | 32.304 | 1.00 | 36.04 | A |
| ATOM | 2283 | NH1 | ARG | A | 304 | 18.501 | 59.518 | 32.000 | 1.00 | 36.26 | A |
| ATOM | 2284 | NH2 | ARG | A | 304 | 17.886 | 57.386 | 31.419 | 1.00 | 37.33 | A |
| ATOM | 2285 | C | ARG | A | 304 | 24.305 | 57.958 | 33.789 | 1.00 | 24.35 | A |
| ATOM | 2286 | O | ARG | A | 304 | 24.503 | 56.858 | 33.276 | 1.00 | 23.88 | A |
| ATOM | 2287 | N | THR | A | 305 | 25.102 | 58.487 | 34.710 | 1.00 | 23.66 | A |
| ATOM | 2288 | CA | THR | A | 305 | 26.276 | 57.779 | 35.194 | 1.00 | 23.22 | A |
| ATOM | 2289 | CB | THR | A | 305 | 26.918 | 58.528 | 36.375 | 1.00 | 23.98 | A |
| ATOM | 2290 | OG1 | THR | A | 305 | 27.347 | 59.823 | 35.934 | 1.00 | 25.33 | A |
| ATOM | 2291 | CG2 | THR | A | 305 | 28.106 | 57.759 | 36.920 | 1.00 | 23.64 | A |
| ATOM | 2292 | C | THR | A | 305 | 25.866 | 56.385 | 35.643 | 1.00 | 22.81 | A |
| ATOM | 2293 | O | THR | A | 305 | 24.868 | 56.224 | 36.346 | 1.00 | 22.49 | A |
| ATOM | 2294 | N | ILE | A | 306 | 26.628 | 55.378 | 35.227 | 1.00 | 22.00 | A |
| ATOM | 2295 | CA | ILE | A | 306 | 26.327 | 54.000 | 35.597 | 1.00 | 21.93 | A |
| ATOM | 2296 | CB | ILE | A | 306 | 27.073 | 52.991 | 34.696 | 1.00 | 21.54 | A |
| ATOM | 2297 | CG2 | ILE | A | 306 | 26.633 | 51.565 | 35.035 | 1.00 | 21.28 | A |
| ATOM | 2298 | CG1 | ILE | A | 306 | 26.803 | 53.301 | 33.220 | 1.00 | 20.62 | A |
| ATOM | 2299 | CD1 | ILE | A | 306 | 25.337 | 53.240 | 32.822 | 1.00 | 20.14 | A |
| ATOM | 2300 | C | ILE | A | 306 | 26.734 | 53.747 | 37.046 | 1.00 | 22.59 | A |
| ATOM | 2301 | O | ILE | A | 306 | 27.800 | 54.180 | 37.487 | 1.00 | 21.84 | A |
| ATOM | 2302 | N | SER | A | 307 | 25.875 | 53.044 | 37.779 | 1.00 | 23.35 | A |
| ATOM | 2303 | CA | SER | A | 307 | 26.132 | 52.716 | 39.178 | 1.00 | 24.13 | A |
| ATOM | 2304 | CB | SER | A | 307 | 25.371 | 53.675 | 40.094 | 1.00 | 23.72 | A |
| ATOM | 2305 | OG | SER | A | 307 | 23.973 | 53.550 | 39.900 | 1.00 | 23.17 | A |
| ATOM | 2306 | C | SER | A | 307 | 25.659 | 51.292 | 39.443 | 1.00 | 24.92 | A |
| ATOM | 2307 | O | SER | A | 307 | 24.927 | 50.721 | 38.639 | 1.00 | 24.44 | A |
| ATOM | 2308 | N | ASP | A | 308 | 26.079 | 50.719 | 40.567 | 1.00 | 26.08 | A |
| ATOM | 2309 | CA | ASP | A | 308 | 25.669 | 49.363 | 40.922 | 1.00 | 27.21 | A |
| ATOM | 2310 | CB | ASP | A | 308 | 26.296 | 48.955 | 42.257 | 1.00 | 28.74 | A |
| ATOM | 2311 | CG | ASP | A | 308 | 27.785 | 48.707 | 42.148 | 1.00 | 30.44 | A |
| ATOM | 2312 | OD1 | ASP | A | 308 | 28.394 | 49.166 | 41.160 | 1.00 | 31.50 | A |
| ATOM | 2313 | OD2 | ASP | A | 308 | 28.351 | 48.060 | 43.055 | 1.00 | 32.13 | A |
| ATOM | 2314 | C | ASP | A | 308 | 24.152 | 49.325 | 41.040 | 1.00 | 26.58 | A |
| ATOM | 2315 | O | ASP | A | 308 | 23.522 | 48.284 | 40.858 | 1.00 | 26.24 | A |
| ATOM | 2316 | N | GLN | A | 309 | 23.582 | 50.487 | 41.331 | 1.00 | 26.55 | A |
| ATOM | 2317 | CA | GLN | A | 309 | 22.147 | 50.651 | 41.504 | 1.00 | 26.62 | A |
| ATOM | 2318 | CB | GLN | A | 309 | 21.899 | 51.936 | 42.306 | 1.00 | 28.47 | A |
| ATOM | 2319 | CG | GLN | A | 309 | 20.524 | 52.551 | 42.178 | 1.00 | 30.34 | A |
| ATOM | 2320 | CD | GLN | A | 309 | 20.373 | 53.799 | 43.039 | 1.00 | 32.12 | A |
| ATOM | 2321 | OE1 | GLN | A | 309 | 19.548 | 54.672 | 42.757 | 1.00 | 32.61 | A |
| ATOM | 2322 | NE2 | GLN | A | 309 | 21.166 | 53.881 | 44.102 | 1.00 | 32.15 | A |
| ATOM | 2323 | C | GLN | A | 309 | 21.331 | 50.654 | 40.210 | 1.00 | 25.60 | A |
| ATOM | 2324 | O | GLN | A | 309 | 20.210 | 50.148 | 40.189 | 1.00 | 26.28 | A |
| ATOM | 2325 | N | ASN | A | 310 | 21.881 | 51.205 | 39.131 | 1.00 | 23.60 | A |
| ATOM | 2326 | CA | ASN | A | 310 | 21.142 | 51.251 | 37.870 | 1.00 | 22.29 | A |
| ATOM | 2327 | CB | ASN | A | 310 | 20.991 | 52.700 | 37.388 | 1.00 | 21.33 | A |
| ATOM | 2328 | CG | ASN | A | 310 | 22.324 | 53.345 | 37.034 | 1.00 | 20.86 | A |
| ATOM | 2329 | OD1 | ASN | A | 310 | 23.260 | 52.670 | 36.606 | 1.00 | 19.45 | A |
| ATOM | 2330 | ND2 | ASN | A | 310 | 22.407 | 54.661 | 37.195 | 1.00 | 20.66 | A |
| ATOM | 2331 | C | ASN | A | 310 | 21.744 | 50.429 | 36.734 | 1.00 | 21.87 | A |
| ATOM | 2332 | O | ASN | A | 310 | 21.162 | 50.356 | 35.656 | 1.00 | 21.72 | A |
| ATOM | 2333 | N | VAL | A | 311 | 22.897 | 49.811 | 36.970 | 1.00 | 21.87 | A |
| ATOM | 2334 | CA | VAL | A | 311 | 23.572 | 49.035 | 35.929 | 1.00 | 21.29 | A |
| ATOM | 2335 | CB | VAL | A | 311 | 24.907 | 48.445 | 36.446 | 1.00 | 21.36 | A |
| ATOM | 2336 | CG1 | VAL | A | 311 | 24.639 | 47.333 | 37.448 | 1.00 | 21.68 | A |
| ATOM | 2337 | CG2 | VAL | A | 311 | 25.746 | 47.940 | 35.270 | 1.00 | 20.87 | A |
| ATOM | 2338 | C | VAL | A | 311 | 22.741 | 47.912 | 35.302 | 1.00 | 21.68 | A |
| ATOM | 2339 | O | VAL | A | 311 | 22.857 | 47.652 | 34.104 | 1.00 | 20.40 | A |
| ATOM | 2340 | N | ALA | A | 312 | 21.906 | 47.247 | 36.095 | 1.00 | 20.96 | A |
| ATOM | 2341 | CA | ALA | A | 312 | 21.083 | 46.167 | 35.557 | 1.00 | 21.12 | A |
| ATOM | 2342 | CB | ALA | A | 312 | 20.368 | 45.424 | 36.692 | 1.00 | 21.66 | A |
| ATOM | 2343 | C | ALA | A | 312 | 20.065 | 46.698 | 34.552 | 1.00 | 21.29 | A |
| ATOM | 2344 | O | ALA | A | 312 | 19.860 | 46.103 | 33.493 | 1.00 | 21.31 | A |
| ATOM | 2345 | N | ALA | A | 313 | 19.431 | 47.821 | 34.885 | 1.00 | 21.28 | A |
| ATOM | 2346 | CA | ALA | A | 313 | 18.431 | 48.434 | 34.014 | 1.00 | 21.25 | A |
| ATOM | 2347 | CB | ALA | A | 313 | 17.637 | 49.478 | 34.785 | 1.00 | 20.73 | A |
| ATOM | 2348 | C | ALA | A | 313 | 19.078 | 49.077 | 32.789 | 1.00 | 21.41 | A |
| ATOM | 2349 | O | ALA | A | 313 | 18.547 | 49.003 | 31.679 | 1.00 | 20.99 | A |
| ATOM | 2350 | N | ARG | A | 314 | 20.218 | 49.722 | 33.000 | 1.00 | 21.35 | A |
| ATOM | 2351 | CA | ARG | A | 314 | 20.934 | 50.365 | 31.901 | 1.00 | 21.81 | A |
| ATOM | 2352 | CB | ARG | A | 314 | 22.138 | 51.139 | 32.444 | 1.00 | 21.63 | A |
| ATOM | 2353 | CG | ARG | A | 314 | 21.790 | 52.300 | 33.368 | 1.00 | 21.97 | A |
| ATOM | 2354 | CD | ARG | A | 314 | 21.440 | 53.566 | 32.596 | 1.00 | 22.71 | A |
| ATOM | 2355 | NE | ARG | A | 314 | 20.076 | 53.563 | 32.081 | 1.00 | 23.17 | A |
| ATOM | 2356 | CZ | ARG | A | 314 | 19.589 | 54.488 | 31.260 | 1.00 | 23.56 | A |
| ATOM | 2357 | NH1 | ARG | A | 314 | 20.357 | 55.490 | 30.854 | 1.00 | 24.71 | A |
| ATOM | 2358 | NH2 | ARG | A | 314 | 18.330 | 54.422 | 30.855 | 1.00 | 23.76 | A |
| ATOM | 2359 | C | ARG | A | 314 | 21.406 | 49.299 | 30.911 | 1.00 | 21.67 | A |
| ATOM | 2360 | O | ARG | A | 314 | 21.304 | 49.476 | 29.695 | 1.00 | 21.54 | A |
| ATOM | 2361 | N | SER | A | 315 | 21.912 | 48.190 | 31.441 | 1.00 | 21.71 | A |
| ATOM | 2362 | CA | SER | A | 315 | 22.399 | 47.088 | 30.612 | 1.00 | 22.55 | A |
| ATOM | 2363 | CB | SER | A | 315 | 23.062 | 46.016 | 31.481 | 1.00 | 21.53 | A |
| ATOM | 2364 | OG | SER | A | 315 | 24.246 | 46.507 | 32.082 | 1.00 | 21.43 | A |
| ATOM | 2365 | C | SER | A | 315 | 21.266 | 46.461 | 29.815 | 1.00 | 23.17 | A |
| ATOM | 2366 | O | SER | A | 315 | 21.424 | 46.141 | 28.637 | 1.00 | 23.47 | A |
| ATOM | 2367 | N | ASP | A | 316 | 20.118 | 46.287 | 30.459 | 1.00 | 23.63 | A |
| ATOM | 2368 | CA | ASP | A | 316 | 18.971 | 45.694 | 29.789 | 1.00 | 24.11 | A |
| ATOM | 2369 | CB | ASP | A | 316 | 17.790 | 45.597 | 30.759 | 1.00 | 26.34 | A |
| ATOM | 2370 | CG | ASP | A | 316 | 16.723 | 44.632 | 30.283 | 1.00 | 28.47 | A |
| ATOM | 2371 | OD1 | ASP | A | 316 | 17.060 | 43.467 | 29.973 | 1.00 | 29.91 | A |
| ATOM | 2372 | OD2 | ASP | A | 316 | 15.544 | 45.037 | 30.226 | 1.00 | 29.93 | A |
| ATOM | 2373 | C | ASP | A | 316 | 18.604 | 46.543 | 28.573 | 1.00 | 23.57 | A |
| ATOM | 2374 | O | ASP | A | 316 | 18.309 | 46.012 | 27.502 | 1.00 | 23.16 | A |
| ATOM | 2375 | N | LEU | A | 317 | 18.635 | 47.863 | 28.737 | 1.00 | 22.39 | A |
| ATOM | 2376 | CA | LEU | A | 317 | 18.321 | 48.769 | 27.637 | 1.00 | 21.62 | A |
| ATOM | 2377 | CB | LEU | A | 317 | 18.253 | 50.217 | 28.132 | 1.00 | 22.62 | A |
| ATOM | 2378 | CG | LEU | A | 317 | 16.882 | 50.779 | 28.506 | 1.00 | 23.63 | A |
| ATOM | 2379 | CD1 | LEU | A | 317 | 17.055 | 52.191 | 29.061 | 1.00 | 24.97 | A |
| ATOM | 2380 | CD2 | LEU | A | 317 | 15.978 | 50.796 | 27.281 | 1.00 | 23.92 | A |
| ATOM | 2381 | C | LEU | A | 317 | 19.364 | 48.682 | 26.529 | 1.00 | 20.23 | A |
| ATOM | 2382 | O | LEU | A | 317 | 19.024 | 48.571 | 25.352 | 1.00 | 19.59 | A |
| ATOM | 2383 | N | LEU | A | 318 | 20.634 | 48.735 | 26.915 | 1.00 | 19.08 | A |
| ATOM | 2384 | CA | LEU | A | 318 | 21.726 | 48.690 | 25.948 | 1.00 | 18.36 | A |
| ATOM | 2385 | CB | LEU | A | 318 | 23.061 | 48.959 | 26.647 | 1.00 | 17.32 | A |
| ATOM | 2386 | CG | LEU | A | 318 | 24.279 | 49.101 | 25.727 | 1.00 | 17.28 | A |
| ATOM | 2387 | CD1 | LEU | A | 318 | 24.040 | 50.232 | 24.732 | 1.00 | 16.94 | A |
| ATOM | 2388 | CD2 | LEU | A | 318 | 25.527 | 49.370 | 26.563 | 1.00 | 16.17 | A |
| ATOM | 2389 | C | LEU | A | 318 | 21.797 | 47.361 | 25.208 | 1.00 | 18.02 | A |
| ATOM | 2390 | O | LEU | A | 318 | 21.841 | 47.326 | 23.977 | 1.00 | 17.93 | A |
| ATOM | 2391 | N | VAL | A | 319 | 21.816 | 46.265 | 25.958 | 1.00 | 17.30 | A |
| ATOM | 2392 | CA | VAL | A | 319 | 21.883 | 44.948 | 25.343 | 1.00 | 17.29 | A |
| ATOM | 2393 | CB | VAL | A | 319 | 21.822 | 43.835 | 26.408 | 1.00 | 17.01 | A |
| ATOM | 2394 | CG1 | VAL | A | 319 | 21.644 | 42.479 | 25.742 | 1.00 | 17.97 | A |
| ATOM | 2395 | CG2 | VAL | A | 319 | 23.103 | 43.843 | 27.232 | 1.00 | 17.03 | A |
| ATOM | 2396 | C | VAL | A | 319 | 20.743 | 44.768 | 24.345 | 1.00 | 16.94 | A |
| ATOM | 2397 | O | VAL | A | 319 | 20.925 | 44.167 | 23.289 | 1.00 | 17.15 | A |
| ATOM | 2398 | N | ASP | A | 320 | 19.571 | 45.301 | 24.671 | 1.00 | 16.74 | A |
| ATOM | 2399 | CA | ASP | A | 320 | 18.422 | 45.186 | 23.776 | 1.00 | 16.82 | A |
| ATOM | 2400 | CB | ASP | A | 320 | 17.181 | 45.794 | 24.433 | 1.00 | 18.14 | A |
| ATOM | 2401 | CG | ASP | A | 320 | 15.978 | 45.788 | 23.521 | 1.00 | 17.78 | A |
| ATOM | 2402 | OD1 | ASP | A | 320 | 15.529 | 46.885 | 23.129 | 1.00 | 18.82 | A |
| ATOM | 2403 | OD2 | ASP | A | 320 | 15.482 | 44.690 | 23.195 | 1.00 | 19.23 | A |
| ATOM | 2404 | C | ASP | A | 320 | 18.717 | 45.878 | 22.444 | 1.00 | 16.65 | A |
| ATOM | 2405 | O | ASP | A | 320 | 18.351 | 45.380 | 21.379 | 1.00 | 15.66 | A |
| ATOM | 2406 | N | GLN | A | 321 | 19.377 | 47.031 | 22.508 | 1.00 | 16.01 | A |
| ATOM | 2407 | CA | GLN | A | 321 | 19.741 | 47.762 | 21.297 | 1.00 | 15.56 | A |
| ATOM | 2408 | CB | GLN | A | 321 | 20.367 | 49.113 | 21.652 | 1.00 | 15.14 | A |
| ATOM | 2409 | CG | GLN | A | 321 | 19.358 | 50.150 | 22.095 | 1.00 | 15.99 | A |
| ATOM | 2410 | CD | GLN | A | 321 | 18.322 | 50.421 | 21.027 | 1.00 | 16.42 | A |
| ATOM | 2411 | OE1 | GLN | A | 321 | 18.652 | 50.847 | 19.917 | 1.00 | 15.10 | A |
| ATOM | 2412 | NE2 | GLN | A | 321 | 17.060 | 50.170 | 21.352 | 1.00 | 16.60 | A |
| ATOM | 2413 | C | GLN | A | 321 | 20.742 | 46.937 | 20.501 | 1.00 | 15.56 | A |
| ATOM | 2414 | O | GLN | A | 321 | 20.632 | 46.816 | 19.280 | 1.00 | 15.19 | A |
| ATOM | 2415 | N | TRP | A | 322 | 21.722 | 46.372 | 21.203 | 1.00 | 15.41 | A |
| ATOM | 2416 | CA | TRP | A | 322 | 22.742 | 45.550 | 20.565 | 1.00 | 15.95 | A |
| ATOM | 2417 | CB | TRP | A | 322 | 23.751 | 45.031 | 21.594 | 1.00 | 15.32 | A |
| ATOM | 2418 | CG | TRP | A | 322 | 24.698 | 46.063 | 22.138 | 1.00 | 16.14 | A |
| ATOM | 2419 | CD2 | TRP | A | 322 | 25.591 | 45.900 | 23.246 | 1.00 | 16.10 | A |
| ATOM | 2420 | CE2 | TRP | A | 322 | 26.331 | 47.097 | 23.369 | 1.00 | 16.22 | A |
| ATOM | 2421 | CE3 | TRP | A | 322 | 25.839 | 44.856 | 24.148 | 1.00 | 16.86 | A |
| ATOM | 2422 | CD1 | TRP | A | 322 | 24.922 | 47.316 | 21.649 | 1.00 | 16.34 | A |
| ATOM | 2423 | NE1 | TRP | A | 322 | 25.904 | 47.945 | 22.382 | 1.00 | 16.27 | A |
| ATOM | 2424 | CZ2 | TRP | A | 322 | 27.303 | 47.280 | 24.360 | 1.00 | 16.63 | A |
| ATOM | 2425 | CZ3 | TRP | A | 322 | 26.807 | 45.037 | 25.134 | 1.00 | 16.10 | A |
| ATOM | 2426 | CH2 | TRP | A | 322 | 27.526 | 46.240 | 25.231 | 1.00 | 16.78 | A |
| ATOM | 2427 | C | TRP | A | 322 | 22.136 | 44.356 | 19.834 | 1.00 | 16.03 | A |
| ATOM | 2428 | O | TRP | A | 322 | 22.475 | 44.088 | 18.681 | 1.00 | 15.82 | A |
| ATOM | 2429 | N | LYS | A | 323 | 21.249 | 43.632 | 20.510 | 1.00 | 15.91 | A |
| ATOM | 2430 | CA | LYS | A | 323 | 20.624 | 42.466 | 19.897 | 1.00 | 16.68 | A |
| ATOM | 2431 | CB | LYS | A | 323 | 19.824 | 41.683 | 20.942 | 1.00 | 16.48 | A |
| ATOM | 2432 | CG | LYS | A | 323 | 20.741 | 40.900 | 21.882 | 1.00 | 17.56 | A |
| ATOM | 2433 | CD | LYS | A | 323 | 19.971 | 40.046 | 22.879 | 1.00 | 19.27 | A |
| ATOM | 2434 | CE | LYS | A | 323 | 20.935 | 39.208 | 23.709 | 1.00 | 19.50 | A |
| ATOM | 2435 | NZ | LYS | A | 323 | 20.226 | 38.348 | 24.692 | 1.00 | 21.53 | A |
| ATOM | 2436 | C | LYS | A | 323 | 19.759 | 42.833 | 18.699 | 1.00 | 16.16 | A |
| ATOM | 2437 | O | LYS | A | 323 | 19.619 | 42.046 | 17.766 | 1.00 | 16.35 | A |
| ATOM | 2438 | N | LYS | A | 324 | 19.183 | 44.029 | 18.713 | 1.00 | 16.26 | A |
| ATOM | 2439 | CA | LYS | A | 324 | 18.377 | 44.460 | 17.579 | 1.00 | 15.66 | A |
| ATOM | 2440 | CB | LYS | A | 324 | 17.549 | 45.694 | 17.943 | 1.00 | 15.63 | A |
| ATOM | 2441 | CG | LYS | A | 324 | 16.381 | 45.363 | 18.866 | 1.00 | 17.11 | A |
| ATOM | 2442 | CD | LYS | A | 324 | 15.589 | 46.597 | 19.275 | 1.00 | 17.10 | A |
| ATOM | 2443 | CE | LYS | A | 324 | 14.397 | 46.197 | 20.148 | 1.00 | 17.89 | A |
| ATOM | 2444 | NZ | LYS | A | 324 | 13.676 | 47.384 | 20.691 | 1.00 | 18.13 | A |
| ATOM | 2445 | C | LYS | A | 324 | 19.312 | 44.757 | 16.412 | 1.00 | 15.66 | A |
| ATOM | 2446 | O | LYS | A | 324 | 19.040 | 44.376 | 15.273 | 1.00 | 15.11 | A |
| ATOM | 2447 | N | LYS | A | 325 | 20.429 | 45.422 | 16.696 | 1.00 | 14.87 | A |
| ATOM | 2448 | CA | LYS | A | 325 | 21.391 | 45.733 | 15.643 | 1.00 | 14.73 | A |
| ATOM | 2449 | CB | LYS | A | 325 | 22.545 | 46.579 | 16.198 | 1.00 | 13.74 | A |
| ATOM | 2450 | CG | LYS | A | 325 | 23.442 | 47.177 | 15.118 | 1.00 | 13.46 | A |
| ATOM | 2451 | CD | LYS | A | 325 | 24.584 | 48.006 | 15.707 | 1.00 | 12.93 | A |
| ATOM | 2452 | CE | LYS | A | 325 | 25.403 | 48.663 | 14.600 | 1.00 | 13.37 | A |
| ATOM | 2453 | NZ | LYS | A | 325 | 26.596 | 49.381 | 15.126 | 1.00 | 13.83 | A |
| ATOM | 2454 | C | LYS | A | 325 | 21.938 | 44.422 | 15.074 | 1.00 | 15.45 | A |
| ATOM | 2455 | O | LYS | A | 325 | 22.105 | 44.279 | 13.862 | 1.00 | 15.46 | A |
| ATOM | 2456 | N | ALA | A | 326 | 22.202 | 43.464 | 15.961 | 1.00 | 15.77 | A |
| ATOM | 2457 | CA | ALA | A | 326 | 22.733 | 42.159 | 15.570 | 1.00 | 16.36 | A |
| ATOM | 2458 | CB | ALA | A | 326 | 22.977 | 41.306 | 16.812 | 1.00 | 16.30 | A |
| ATOM | 2459 | C | ALA | A | 326 | 21.835 | 41.400 | 14.589 | 1.00 | 16.25 | A |
| ATOM | 2460 | O | ALA | A | 326 | 22.311 | 40.550 | 13.834 | 1.00 | 16.73 | A |
| ATOM | 2461 | N | GLU | A | 327 | 20.541 | 41.697 | 14.605 | 1.00 | 16.39 | A |
| ATOM | 2462 | CA | GLU | A | 327 | 19.601 | 41.030 | 13.706 | 1.00 | 16.62 | A |
| ATOM | 2463 | CB | GLU | A | 327 | 18.162 | 41.372 | 14.090 | 1.00 | 17.67 | A |
| ATOM | 2464 | CG | GLU | A | 327 | 17.628 | 40.572 | 15.251 | 1.00 | 19.60 | A |
| ATOM | 2465 | CD | GLU | A | 327 | 17.582 | 39.087 | 14.945 | 1.00 | 19.61 | A |
| ATOM | 2466 | OE1 | GLU | A | 327 | 16.906 | 38.699 | 13.970 | 1.00 | 20.21 | A |
| ATOM | 2467 | OE2 | GLU | A | 327 | 18.224 | 38.312 | 15.678 | 1.00 | 19.93 | A |
| ATOM | 2468 | C | GLU | A | 327 | 19.820 | 41.409 | 12.247 | 1.00 | 16.26 | A |
| ATOM | 2469 | O | GLU | A | 327 | 19.375 | 40.705 | 11.341 | 1.00 | 16.58 | A |
| ATOM | 2470 | N | LEU | A | 328 | 20.505 | 42.525 | 12.022 | 1.00 | 15.87 | A |
| ATOM | 2471 | CA | LEU | A | 328 | 20.749 | 42.991 | 10.667 | 1.00 | 14.87 | A |
| ATOM | 2472 | CB | LEU | A | 328 | 20.909 | 44.517 | 10.660 | 1.00 | 14.25 | A |
| ATOM | 2473 | CG | LEU | A | 328 | 19.804 | 45.322 | 11.357 | 1.00 | 14.52 | A |
| ATOM | 2474 | CD1 | LEU | A | 328 | 20.035 | 46.811 | 11.130 | 1.00 | 13.79 | A |
| ATOM | 2475 | CD2 | LEU | A | 328 | 18.435 | 44.905 | 10.826 | 1.00 | 14.50 | A |
| ATOM | 2476 | C | LEU | A | 328 | 21.968 | 42.337 | 10.026 | 1.00 | 14.72 | A |
| ATOM | 2477 | O | LEU | A | 328 | 22.224 | 42.542 | 8.840 | 1.00 | 15.39 | A |
| ATOM | 2478 | N | TYR | A | 329 | 22.713 | 41.550 | 10.803 | 1.00 | 14.33 | A |
| ATOM | 2479 | CA | TYR | A | 329 | 23.906 | 40.874 | 10.292 | 1.00 | 14.79 | A |
| ATOM | 2480 | CB | TYR | A | 329 | 25.164 | 41.451 | 10.953 | 1.00 | 14.59 | A |
| ATOM | 2481 | CG | TYR | A | 329 | 25.358 | 42.921 | 10.635 | 1.00 | 14.92 | A |
| ATOM | 2482 | CD1 | TYR | A | 329 | 24.740 | 43.912 | 11.404 | 1.00 | 14.49 | A |
| ATOM | 2483 | CE1 | TYR | A | 329 | 24.861 | 45.262 | 11.075 | 1.00 | 14.49 | A |
| ATOM | 2484 | CD2 | TYR | A | 329 | 26.107 | 43.322 | 9.526 | 1.00 | 14.27 | A |
| ATOM | 2485 | CE2 | TYR | A | 329 | 26.233 | 44.668 | 9.188 | 1.00 | 14.38 | A |
| ATOM | 2486 | CZ | TYR | A | 329 | 25.608 | 45.632 | 9.966 | 1.00 | 14.49 | A |
| ATOM | 2487 | OH | TYR | A | 329 | 25.722 | 46.963 | 9.632 | 1.00 | 14.95 | A |
| ATOM | 2488 | C | TYR | A | 329 | 23.844 | 39.351 | 10.451 | 1.00 | 15.24 | A |
| ATOM | 2489 | O | TYR | A | 329 | 22.958 | 38.826 | 11.131 | 1.00 | 15.30 | A |
| ATOM | 2490 | N | ARG | A | 330 | 24.792 | 38.649 | 9.833 | 1.00 | 15.25 | A |
| ATOM | 2491 | CA | ARG | A | 330 | 24.797 | 37.189 | 9.841 | 1.00 | 15.68 | A |
| ATOM | 2492 | CB | ARG | A | 330 | 25.167 | 36.688 | 8.439 | 1.00 | 15.44 | A |
| ATOM | 2493 | CG | ARG | A | 330 | 24.273 | 37.268 | 7.350 | 1.00 | 15.40 | A |
| ATOM | 2494 | CD | ARG | A | 330 | 24.497 | 36.617 | 5.990 | 1.00 | 15.70 | A |
| ATOM | 2495 | NE | ARG | A | 330 | 23.578 | 37.185 | 5.008 | 1.00 | 16.37 | A |
| ATOM | 2496 | CZ | ARG | A | 330 | 23.309 | 36.645 | 3.823 | 1.00 | 17.09 | A |
| ATOM | 2497 | NH1 | ARG | A | 330 | 23.889 | 35.509 | 3.454 | 1.00 | 16.94 | A |
| ATOM | 2498 | NH2 | ARG | A | 330 | 22.446 | 37.239 | 3.007 | 1.00 | 16.97 | A |
| ATOM | 2499 | C | ARG | A | 330 | 23.618 | 36.416 | 10.876 | 1.00 | 16.06 | A |
| ATOM | 2500 | O | ARG | A | 330 | 25.376 | 35.226 | 11.071 | 1.00 | 16.42 | A |
| ATOM | 2501 | N | THR | A | 331 | 26.586 | 37.053 | 11.528 | 1.00 | 16.07 | A |
| ATOM | 2502 | CA | THR | A | 331 | 27.382 | 36.329 | 12.521 | 1.00 | 15.58 | A |
| ATOM | 2503 | CB | THR | A | 331 | 28.900 | 36.578 | 12.352 | 1.00 | 15.84 | A |
| ATOM | 2504 | OG1 | THR | A | 331 | 29.222 | 37.895 | 12.813 | 1.00 | 14.68 | A |
| ATOM | 2505 | CG2 | THR | A | 331 | 29.314 | 36.436 | 10.888 | 1.00 | 15.90 | A |
| ATOM | 2506 | C | THR | A | 331 | 27.005 | 36.735 | 13.938 | 1.00 | 15.84 | A |
| ATOM | 2507 | O | THR | A | 331 | 26.157 | 37.607 | 14.141 | 1.00 | 15.86 | A |
| ATOM | 2508 | N | ASN | A | 332 | 27.646 | 36.100 | 14.915 | 1.00 | 16.04 | A |
| ATOM | 2509 | CA | ASN | A | 332 | 27.394 | 36.403 | 16.318 | 1.00 | 16.70 | A |
| ATOM | 2510 | CB | ASN | A | 332 | 27.380 | 35.108 | 17.151 | 1.00 | 17.69 | A |
| ATOM | 2511 | CG | ASN | A | 332 | 28.749 | 34.452 | 17.251 | 1.00 | 19.65 | A |
| ATOM | 2512 | OD1 | ASN | A | 332 | 29.525 | 34.447 | 16.295 | 1.00 | 19.62 | A |
| ATOM | 2513 | ND2 | ASN | A | 332 | 29.043 | 33.876 | 18.416 | 1.00 | 20.32 | A |
| ATOM | 2514 | C | ASN | A | 332 | 28.458 | 37.373 | 16.837 | 1.00 | 16.36 | A |
| ATOM | 2515 | O | ASN | A | 332 | 28.714 | 37.454 | 18.041 | 1.00 | 16.17 | A |
| ATOM | 2516 | N | VAL | A | 333 | 29.076 | 38.106 | 15.911 | 1.00 | 15.18 | A |
| ATOM | 2517 | CA | VAL | A | 333 | 30.095 | 39.094 | 16.253 | 1.00 | 14.95 | A |
| ATOM | 2518 | CB | VAL | A | 333 | 31.409 | 38.846 | 15.476 | 1.00 | 15.34 | A |
| ATOM | 2519 | CG1 | VAL | A | 333 | 32.449 | 39.892 | 15.866 | 1.00 | 15.33 | A |
| ATOM | 2520 | CG2 | VAL | A | 333 | 31.926 | 37.448 | 15.767 | 1.00 | 15.82 | A |
| ATOM | 2521 | C | VAL | A | 333 | 29.520 | 40.453 | 15.857 | 1.00 | 14.77 | A |
| ATOM | 2522 | O | VAL | A | 333 | 29.192 | 40.674 | 14.691 | 1.00 | 15.06 | A |
| ATOM | 2523 | N | LEU | A | 334 | 29.406 | 41.361 | 16.823 | 1.00 | 13.87 | A |
| ATOM | 2524 | CA | LEU | A | 334 | 28.814 | 42.670 | 16.568 | 1.00 | 13.41 | A |
| ATOM | 2525 | CB | LEU | A | 334 | 27.608 | 42.860 | 17.494 | 1.00 | 13.88 | A |
| ATOM | 2526 | CG | LEU | A | 334 | 26.789 | 44.140 | 17.337 | 1.00 | 13.89 | A |
| ATOM | 2527 | CD1 | LEU | A | 334 | 26.070 | 44.123 | 15.992 | 1.00 | 14.77 | A |
| ATOM | 2528 | CD2 | LEU | A | 334 | 25.787 | 44.244 | 18.479 | 1.00 | 13.69 | A |
| ATOM | 2529 | C | LEU | A | 334 | 29.758 | 43.865 | 16.720 | 1.00 | 13.04 | A |
| ATOM | 2530 | O | LEU | A | 334 | 30.475 | 43.981 | 17.713 | 1.00 | 12.82 | A |
| ATOM | 2531 | N | LEU | A | 335 | 29.735 | 44.758 | 15.735 | 1.00 | 12.97 | A |
| ATOM | 2532 | CA | LEU | A | 335 | 30.579 | 45.954 | 15.759 | 1.00 | 12.73 | A |
| ATOM | 2533 | CB | LEU | A | 335 | 31.079 | 46.285 | 14.349 | 1.00 | 12.26 | A |
| ATOM | 2534 | CG | LEU | A | 335 | 31.843 | 47.608 | 14.216 | 1.00 | 12.34 | A |
| ATOM | 2535 | CD1 | LEU | A | 335 | 33.151 | 47.533 | 14.988 | 1.00 | 12.28 | A |
| ATOM | 2536 | CD2 | LEU | A | 335 | 32.107 | 47.900 | 12.749 | 1.00 | 12.78 | A |
| ATOM | 2537 | C | LEU | A | 335 | 29.795 | 47.144 | 16.299 | 1.00 | 12.79 | A |
| ATOM | 2538 | O | LEU | A | 335 | 28.723 | 47.466 | 15.793 | 1.00 | 13.65 | A |
| ATOM | 2539 | N | ILE | A | 336 | 30.337 | 47.797 | 17.324 | 1.00 | 12.68 | A |
| ATOM | 2540 | CA | ILE | A | 336 | 29.694 | 43.961 | 17.925 | 1.00 | 12.47 | A |
| ATOM | 2541 | CB | ILE | A | 336 | 29.181 | 48.659 | 19.366 | 1.00 | 11.61 | A |
| ATOM | 2542 | CG2 | ILE | A | 336 | 28.538 | 49.904 | 19.969 | 1.00 | 12.59 | A |
| ATOM | 2543 | CG1 | ILE | A | 336 | 28.157 | 47.516 | 19.342 | 1.00 | 12.32 | A |
| ATOM | 2544 | CD1 | ILE | A | 336 | 26.853 | 47.851 | 18.625 | 1.00 | 12.23 | A |
| ATOM | 2545 | C | ILE | A | 336 | 30.679 | 50.127 | 18.003 | 1.00 | 12.15 | A |
| ATOM | 2546 | O | ILE | A | 336 | 31.429 | 50.253 | 18.965 | 1.00 | 12.42 | A |
| ATOM | 2547 | N | PRO | A | 337 | 30.710 | 50.981 | 16.970 | 1.00 | 12.21 | A |
| ATOM | 2548 | CD | PRO | A | 337 | 30.050 | 50.890 | 15.657 | 1.00 | 11.90 | A |
| ATOM | 2549 | CA | PRO | A | 337 | 31.636 | 52.118 | 17.017 | 1.00 | 12.00 | A |
| ATOM | 2550 | CB | PRO | A | 337 | 31.406 | 52.809 | 15.674 | 1.00 | 11.62 | A |
| ATOM | 2551 | CG | PRO | A | 337 | 30.989 | 51.687 | 14.776 | 1.00 | 11.75 | A |
| ATOM | 2552 | C | PRO | A | 337 | 31.255 | 53.032 | 18.181 | 1.00 | 11.64 | A |
| ATOM | 2553 | O | PRO | A | 337 | 30.079 | 53.141 | 18.521 | 1.00 | 12.05 | A |
| ATOM | 2554 | N | LEU | A | 338 | 32.247 | 53.678 | 18.789 | 1.00 | 11.36 | A |
| ATOM | 2555 | CA | LEU | A | 338 | 31.991 | 54.601 | 19.894 | 1.00 | 12.19 | A |
| ATOM | 2556 | CB | LEU | A | 338 | 32.392 | 53.982 | 21.239 | 1.00 | 11.63 | A |
| ATOM | 2557 | CG | LEU | A | 338 | 32.104 | 54.867 | 22.461 | 1.00 | 12.93 | A |
| ATOM | 2558 | CD1 | LEU | A | 338 | 30.602 | 54.924 | 22.705 | 1.00 | 12.80 | A |
| ATOM | 2559 | CD2 | LEU | A | 338 | 32.820 | 54.318 | 23.695 | 1.00 | 12.39 | A |
| ATOM | 2560 | C | LEU | A | 338 | 32.800 | 55.874 | 19.670 | 1.00 | 12.31 | A |
| ATOM | 2561 | O | LEU | A | 338 | 33.941 | 55.967 | 20.107 | 1.00 | 13.27 | A |
| ATOM | 2562 | N | GLY | A | 339 | 32.208 | 56.850 | 18.987 | 1.00 | 12.98 | A |
| ATOM | 2563 | CA | GLY | A | 339 | 32.922 | 58.092 | 18.734 | 1.00 | 12.65 | A |
| ATOM | 2564 | C | GLY | A | 339 | 32.129 | 59.114 | 17.943 | 1.00 | 13.02 | A |
| ATOM | 2565 | O | GLY | A | 339 | 30.971 | 58.888 | 17.591 | 1.00 | 13.12 | A |
| ATOM | 2566 | N | ASP | A | 340 | 32.768 | 60.245 | 17.660 | 1.00 | 12.52 | A |
| ATOM | 2567 | CA | ASP | A | 340 | 32.141 | 61.331 | 16.921 | 1.00 | 12.74 | A |
| ATOM | 2568 | CB | ASP | A | 340 | 31.162 | 62.075 | 17.831 | 1.00 | 12.87 | A |
| ATOM | 2569 | CG | ASP | A | 340 | 30.042 | 62.758 | 17.065 | 1.00 | 13.84 | A |
| ATOM | 2570 | OD1 | ASP | A | 340 | 30.240 | 63.114 | 15.881 | 1.00 | 14.29 | A |
| ATOM | 2571 | OD2 | ASP | A | 340 | 28.961 | 62.954 | 17.662 | 1.00 | 13.88 | A |
| ATOM | 2572 | C | ASP | A | 340 | 33.265 | 62.273 | 16.475 | 1.00 | 12.96 | A |
| ATOM | 2573 | O | ASP | A | 340 | 34.445 | 61.955 | 16.636 | 1.00 | 12.55 | A |
| ATOM | 2574 | N | ASP | A | 341 | 32.903 | 63.432 | 15.935 | 1.00 | 12.80 | A |
| ATOM | 2575 | CA | ASP | A | 341 | 33.898 | 64.397 | 15.466 | 1.00 | 13.10 | A |
| ATOM | 2576 | CB | ASP | A | 341 | 33.214 | 65.596 | 14.801 | 1.00 | 12.71 | A |
| ATOM | 2577 | CG | ASP | A | 341 | 32.535 | 65.232 | 13.494 | 1.00 | 12.61 | A |
| ATOM | 2578 | OD1 | ASP | A | 341 | 32.480 | 64.029 | 13.170 | 1.00 | 12.74 | A |
| ATOM | 2579 | OD2 | ASP | A | 341 | 32.057 | 66.153 | 12.796 | 1.00 | 12.47 | A |
| ATOM | 2580 | C | ASP | A | 341 | 34.809 | 64.909 | 16.573 | 1.00 | 12.92 | A |
| ATOM | 2581 | O | ASP | A | 341 | 34.341 | 65.381 | 17.611 | 1.00 | 13.34 | A |
| ATOM | 2582 | N | PHE | A | 342 | 36.113 | 64.815 | 16.330 | 1.00 | 12.69 | A |
| ATOM | 2583 | CA | PHE | A | 342 | 37.127 | 65.274 | 17.265 | 1.00 | 12.79 | A |
| ATOM | 2584 | CB | PHE | A | 342 | 37.318 | 66.788 | 17.123 | 1.00 | 12.64 | A |
| ATOM | 2585 | CG | PHE | A | 342 | 37.856 | 67.209 | 15.779 | 1.00 | 12.64 | A |
| ATOM | 2586 | CD1 | PHE | A | 342 | 36.992 | 67.542 | 14.737 | 1.00 | 13.03 | A |
| ATOM | 2587 | CD2 | PHE | A | 342 | 39.230 | 67.258 | 15.551 | 1.00 | 13.23 | A |
| ATOM | 2588 | CE1 | PHE | A | 342 | 37.487 | 67.920 | 13.486 | 1.00 | 12.67 | A |
| ATOM | 2589 | CE2 | PHE | A | 342 | 39.737 | 67.633 | 14.306 | 1.00 | 13.15 | A |
| ATOM | 2590 | CZ | PHE | A | 342 | 38.863 | 67.965 | 13.271 | 1.00 | 13.27 | A |
| ATOM | 2591 | C | PHE | A | 342 | 36.831 | 64.917 | 18.718 | 1.00 | 13.53 | A |
| ATOM | 2592 | O | PHE | A | 342 | 36.970 | 65.752 | 19.618 | 1.00 | 12.91 | A |
| ATOM | 2593 | N | ARG | A | 343 | 36.428 | 63.669 | 18.937 | 1.00 | 13.14 | A |
| ATOM | 2594 | CA | ARG | A | 343 | 36.133 | 63.188 | 20.279 | 1.00 | 14.72 | A |
| ATOM | 2595 | CB | ARG | A | 343 | 35.127 | 62.032 | 20.227 | 1.00 | 14.28 | A |
| ATOM | 2596 | CG | ARG | A | 343 | 33.681 | 62.469 | 20.032 | 1.00 | 15.00 | A |
| ATOM | 2597 | CD | ARG | A | 343 | 33.260 | 63.441 | 21.137 | 1.00 | 14.90 | A |
| ATOM | 2598 | NE | ARG | A | 343 | 31.833 | 63.745 | 21.098 | 1.00 | 15.26 | A |
| ATOM | 2599 | CZ | ARG | A | 343 | 30.891 | 63.011 | 21.682 | 1.00 | 15.55 | A |
| ATOM | 2600 | NH1 | ARG | A | 343 | 31.216 | 61.916 | 22.360 | 1.00 | 13.99 | A |
| ATOM | 2601 | NH2 | ARG | A | 343 | 29.619 | 63.377 | 21.592 | 1.00 | 15.63 | A |
| ATOM | 2602 | C | ARG | A | 343 | 37.398 | 62.727 | 20.994 | 1.00 | 14.93 | A |
| ATOM | 2603 | O | ARG | A | 343 | 38.469 | 62.616 | 20.390 | 1.00 | 14.45 | A |
| ATOM | 2604 | N | PHE | A | 344 | 37.253 | 62.456 | 22.287 | 1.00 | 16.04 | A |
| ATOM | 2605 | CA | PHE | A | 344 | 38.349 | 62.006 | 23.138 | 1.00 | 16.71 | A |
| ATOM | 2606 | CB | PHE | A | 344 | 38.863 | 60.647 | 22.662 | 1.00 | 16.44 | A |
| ATOM | 2607 | CG | PHE | A | 344 | 37.857 | 59.550 | 22.817 | 1.00 | 16.53 | A |
| ATOM | 2608 | CD1 | PHE | A | 344 | 37.093 | 59.128 | 21.735 | 1.00 | 15.84 | A |
| ATOM | 2609 | CD2 | PHE | A | 344 | 37.624 | 58.979 | 24.067 | 1.00 | 16.99 | A |
| ATOM | 2610 | CE1 | PHE | A | 344 | 36.110 | 58.157 | 21.893 | 1.00 | 16.36 | A |
| ATOM | 2611 | CE2 | PHE | A | 344 | 36.643 | 58.006 | 24.235 | 1.00 | 16.62 | A |
| ATOM | 2612 | CZ | PHE | A | 344 | 35.883 | 57.595 | 23.144 | 1.00 | 16.74 | A |
| ATOM | 2613 | C | PHE | A | 344 | 39.484 | 63.009 | 23.223 | 1.00 | 17.76 | A |
| ATOM | 2614 | O | PHE | A | 344 | 40.659 | 62.659 | 23.087 | 1.00 | 17.64 | A |
| ATOM | 2615 | N | LYS | A | 345 | 39.110 | 64.259 | 23.478 | 1.00 | 18.99 | A |
| ATOM | 2616 | CA | LYS | A | 345 | 40.055 | 65.358 | 23.593 | 1.00 | 20.91 | A |
| ATOM | 2617 | CB | LYS | A | 345 | 39.408 | 66.638 | 23.064 | 1.00 | 21.42 | A |
| ATOM | 2618 | CG | LYS | A | 345 | 40.277 | 67.872 | 23.144 | 1.00 | 22.74 | A |
| ATOM | 2619 | CD | LYS | A | 345 | 39.499 | 69.088 | 22.660 | 1.00 | 24.20 | A |
| ATOM | 2620 | CE | LYS | A | 345 | 40.312 | 70.360 | 22.774 | 1.00 | 24.77 | A |
| ATOM | 2621 | NZ | LYS | A | 345 | 39.503 | 71.536 | 22.349 | 1.00 | 26.64 | A |
| ATOM | 2622 | C | LYS | A | 345 | 40.500 | 65.568 | 25.041 | 1.00 | 21.83 | A |
| ATOM | 2623 | O | LYS | A | 345 | 41.691 | 65.570 | 25.333 | 1.00 | 23.15 | A |
| ATOM | 2624 | N | GLN | A | 346 | 39.539 | 65.734 | 25.943 | 1.00 | 22.48 | A |
| ATOM | 2625 | CA | GLN | A | 346 | 39.835 | 65.967 | 27.353 | 1.00 | 23.29 | A |
| ATOM | 2626 | CB | GLN | A | 346 | 38.714 | 66.797 | 27.977 | 1.00 | 24.88 | A |
| ATOM | 2627 | CG | GLN | A | 346 | 38.454 | 68.110 | 27.271 | 1.00 | 27.88 | A |
| ATOM | 2628 | CD | GLN | A | 346 | 37.092 | 68.679 | 27.605 | 1.00 | 29.73 | A |
| ATOM | 2629 | OE1 | GLN | A | 346 | 36.791 | 68.960 | 28.767 | 1.00 | 32.34 | A |
| ATOM | 2630 | NE2 | GLN | A | 346 | 36.253 | 68.847 | 26.585 | 1.00 | 30.40 | A |
| ATOM | 2631 | C | GLN | A | 346 | 40.008 | 64.682 | 28.158 | 1.00 | 22.88 | A |
| ATOM | 2632 | O | GLN | A | 346 | 39.354 | 63.675 | 27.887 | 1.00 | 21.72 | A |
| ATOM | 2633 | N | ASN | A | 347 | 40.887 | 64.725 | 29.156 | 1.00 | 22.84 | A |
| ATOM | 2634 | CA | ASN | A | 347 | 41.121 | 63.566 | 30.012 | 1.00 | 23.28 | A |
| ATOM | 2635 | CB | ASN | A | 347 | 42.124 | 63.903 | 31.120 | 1.00 | 25.05 | A |
| ATOM | 2636 | CG | ASN | A | 347 | 43.495 | 64.219 | 30.584 | 1.00 | 27.25 | A |
| ATOM | 2637 | OD1 | ASN | A | 347 | 44.103 | 63.404 | 29.891 | 1.00 | 28.96 | A |
| ATOM | 2638 | ND2 | ASN | A | 347 | 43.999 | 65.410 | 30.902 | 1.00 | 28.82 | A |
| ATOM | 2639 | C | ASN | A | 347 | 39.804 | 63.156 | 30.651 | 1.00 | 21.95 | A |
| ATOM | 2640 | O | ASN | A | 347 | 39.491 | 61.969 | 30.759 | 1.00 | 21.83 | A |
| ATOM | 2641 | N | THR | A | 348 | 39.037 | 64.154 | 31.077 | 1.00 | 20.97 | A |
| ATOM | 2642 | CA | THR | A | 348 | 37.752 | 63.912 | 31.711 | 1.00 | 20.12 | A |
| ATOM | 2643 | CB | THR | A | 348 | 37.073 | 65.235 | 32.104 | 1.00 | 21.04 | A |
| ATOM | 2644 | OG1 | THR | A | 348 | 37.007 | 66.100 | 30.961 | 1.00 | 22.00 | A |
| ATOM | 2645 | CG2 | THR | A | 348 | 37.857 | 65.921 | 33.216 | 1.00 | 21.79 | A |
| ATOM | 2646 | C | THR | A | 348 | 36.837 | 63.129 | 30.781 | 1.00 | 19.30 | A |
| ATOM | 2647 | O | THR | A | 348 | 36.039 | 62.306 | 31.233 | 1.00 | 18.30 | A |
| ATOM | 2648 | N | GLU | A | 349 | 36.960 | 63.381 | 29.479 | 1.00 | 17.43 | A |
| ATOM | 2649 | CA | GLU | A | 349 | 36.142 | 62.674 | 28.500 | 1.00 | 17.07 | A |
| ATOM | 2650 | CB | GLU | A | 349 | 36.267 | 63.306 | 27.110 | 1.00 | 16.61 | A |
| ATOM | 2651 | CG | GLU | A | 349 | 35.463 | 62.551 | 26.052 | 1.00 | 16.75 | A |
| ATOM | 2652 | CD | GLU | A | 349 | 35.593 | 63.135 | 24.657 | 1.00 | 15.83 | A |
| ATOM | 2653 | OE1 | GLU | A | 349 | 35.037 | 62.529 | 23.717 | 1.00 | 15.90 | A |
| ATOM | 2654 | OE2 | GLU | A | 349 | 36.242 | 64.188 | 24.498 | 1.00 | 16.08 | A |
| ATOM | 2655 | C | GLU | A | 349 | 36.554 | 61.207 | 28.423 | 1.00 | 16.51 | A |
| ATOM | 2656 | O | GLU | A | 349 | 35.700 | 60.323 | 28.379 | 1.00 | 16.14 | A |
| ATOM | 2657 | N | TRP | A | 350 | 37.859 | 60.946 | 28.394 | 1.00 | 16.19 | A |
| ATOM | 2658 | CA | TRP | A | 350 | 38.334 | 59.570 | 28.338 | 1.00 | 16.10 | A |
| ATOM | 2659 | CB | TRP | A | 350 | 39.864 | 59.509 | 28.341 | 1.00 | 15.20 | A |
| ATOM | 2660 | CG | TRP | A | 350 | 40.487 | 59.770 | 27.003 | 1.00 | 15.14 | A |
| ATOM | 2661 | CD2 | TRP | A | 350 | 40.782 | 58.797 | 25.996 | 1.00 | 15.64 | A |
| ATOM | 2662 | CE2 | TRP | A | 350 | 41.322 | 59.490 | 24.888 | 1.00 | 14.56 | A |
| ATOM | 2663 | CE3 | TRP | A | 350 | 40.643 | 57.404 | 25.920 | 1.00 | 15.02 | A |
| ATOM | 2664 | CD1 | TRP | A | 350 | 40.850 | 60.983 | 26.481 | 1.00 | 15.56 | A |
| ATOM | 2665 | NE1 | TRP | A | 350 | 41.352 | 60.821 | 25.212 | 1.00 | 14.85 | A |
| ATOM | 2666 | CZ2 | TRP | A | 350 | 41.719 | 58.837 | 23.718 | 1.00 | 14.95 | A |
| ATOM | 2667 | CZ3 | TRP | A | 350 | 41.039 | 56.754 | 24.754 | 1.00 | 15.06 | A |
| ATOM | 2668 | CH2 | TRP | A | 350 | 41.570 | 57.471 | 23.669 | 1.00 | 15.38 | A |
| ATOM | 2669 | C | TRP | A | 350 | 37.798 | 58.801 | 29.539 | 1.00 | 16.58 | A |
| ATOM | 2670 | O | TRP | A | 350 | 37.298 | 57.683 | 29.404 | 1.00 | 15.92 | A |
| ATOM | 2671 | N | ASP | A | 351 | 37.901 | 59.411 | 30.714 | 1.00 | 17.32 | A |
| ATOM | 2672 | CA | ASP | A | 351 | 37.428 | 58.774 | 31.937 | 1.00 | 18.70 | A |
| ATOM | 2673 | CB | ASP | A | 351 | 37.735 | 59.641 | 33.162 | 1.00 | 19.65 | A |
| ATOM | 2674 | CG | ASP | A | 351 | 39.210 | 59.706 | 33.481 | 1.00 | 21.48 | A |
| ATOM | 2675 | OD1 | ASP | A | 351 | 39.918 | 58.701 | 33.264 | 1.00 | 22.62 | A |
| ATOM | 2676 | OD2 | ASP | A | 351 | 39.656 | 60.762 | 33.970 | 1.00 | 23.82 | A |
| ATOM | 2677 | C | ASP | A | 351 | 35.938 | 58.481 | 31.931 | 1.00 | 18.28 | A |
| ATOM | 2678 | O | ASP | A | 351 | 35.519 | 57.360 | 32.238 | 1.00 | 18.60 | A |
| ATOM | 2679 | N | VAL | A | 352 | 35.134 | 59.484 | 31.589 | 1.00 | 18.03 | A |
| ATOM | 2680 | CA | VAL | A | 352 | 33.691 | 59.309 | 31.606 | 1.00 | 17.92 | A |
| ATOM | 2681 | CB | VAL | A | 352 | 32.958 | 60.655 | 31.351 | 1.00 | 18.50 | A |
| ATOM | 2682 | CG1 | VAL | A | 352 | 32.882 | 60.956 | 29.862 | 1.00 | 18.78 | A |
| ATOM | 2683 | CG2 | VAL | A | 352 | 31.578 | 60.619 | 31.986 | 1.00 | 19.34 | A |
| ATOM | 2684 | C | VAL | A | 352 | 33.198 | 58.236 | 30.633 | 1.00 | 17.67 | A |
| ATOM | 2685 | O | VAL | A | 352 | 32.236 | 57.526 | 30.927 | 1.00 | 16.91 | A |
| ATOM | 2686 | N | GLN | A | 353 | 33.850 | 58.103 | 29.482 | 1.00 | 16.45 | A |
| ATOM | 2687 | CA | GLN | A | 353 | 33.436 | 57.080 | 28.528 | 1.00 | 15.95 | A |
| ATOM | 2688 | CB | GLN | A | 353 | 33.941 | 57.415 | 27.114 | 1.00 | 16.08 | A |
| ATOM | 2689 | CG | GLN | A | 353 | 33.384 | 58.719 | 26.529 | 1.00 | 16.37 | A |
| ATOM | 2690 | CD | GLN | A | 353 | 31.939 | 58.608 | 26.049 | 1.00 | 17.19 | A |
| ATOM | 2691 | OE1 | GLN | A | 353 | 31.114 | 57.935 | 26.666 | 1.00 | 16.64 | A |
| ATOM | 2692 | NE2 | GLN | A | 353 | 31.626 | 59.291 | 24.948 | 1.00 | 16.97 | A |
| ATOM | 2693 | C | GLN | A | 353 | 33.960 | 55.705 | 28.964 | 1.00 | 15.87 | A |
| ATOM | 2694 | O | GLN | A | 353 | 33.206 | 54.732 | 28.994 | 1.00 | 16.13 | A |
| ATOM | 2695 | N | ARG | A | 354 | 35.242 | 55.627 | 29.317 | 1.00 | 15.43 | A |
| ATOM | 2696 | CA | ARG | A | 354 | 35.837 | 54.356 | 29.732 | 1.00 | 15.91 | A |
| ATOM | 2697 | CB | ARG | A | 354 | 37.353 | 54.498 | 29.916 | 1.00 | 15.77 | A |
| ATOM | 2698 | CG | ARG | A | 354 | 38.025 | 53.234 | 30.454 | 1.00 | 16.60 | A |
| ATOM | 2699 | CD | ARG | A | 354 | 39.527 | 53.419 | 30.652 | 1.00 | 17.32 | A |
| ATOM | 2700 | NE | ARG | A | 354 | 39.844 | 54.426 | 31.664 | 1.00 | 18.44 | A |
| ATOM | 2701 | CZ | ARG | A | 354 | 39.642 | 54.276 | 32.971 | 1.00 | 18.97 | A |
| ATOM | 2702 | NH1 | ARG | A | 354 | 39.118 | 53.153 | 33.444 | 1.00 | 18.45 | A |
| ATOM | 2703 | NH2 | ARG | A | 354 | 39.974 | 55.248 | 33.810 | 1.00 | 18.25 | A |
| ATOM | 2704 | C | ARG | A | 354 | 35.247 | 53.756 | 31.006 | 1.00 | 16.32 | A |
| ATOM | 2705 | O | ARG | A | 354 | 34.881 | 52.581 | 31.029 | 1.00 | 15.52 | A |
| ATOM | 2706 | N | VAL | A | 355 | 35.159 | 54.560 | 32.064 | 1.00 | 16.75 | A |
| ATOM | 2707 | CA | VAL | A | 355 | 34.639 | 54.079 | 33.342 | 1.00 | 17.22 | A |
| ATOM | 2708 | CB | VAL | A | 355 | 34.705 | 55.184 | 34.422 | 1.00 | 18.19 | A |
| ATOM | 2709 | CG1 | VAL | A | 355 | 34.034 | 54.706 | 35.705 | 1.00 | 18.85 | A |
| ATOM | 2710 | CG2 | VAL | A | 355 | 36.162 | 55.543 | 34.705 | 1.00 | 18.06 | A |
| ATOM | 2711 | C | VAL | A | 355 | 33.211 | 53.553 | 33.258 | 1.00 | 17.38 | A |
| ATOM | 2712 | O | VAL | A | 355 | 32.913 | 52.457 | 33.742 | 1.00 | 16.87 | A |
| ATOM | 2713 | N | ASN | A | 356 | 32.324 | 54.328 | 32.647 | 1.00 | 16.91 | A |
| ATOM | 2714 | CA | ASN | A | 356 | 30.942 | 53.899 | 32.527 | 1.00 | 16.67 | A |
| ATOM | 2715 | CB | ASN | A | 356 | 30.091 | 55.030 | 31.957 | 1.00 | 16.65 | A |
| ATOM | 2716 | CG | ASN | A | 356 | 29.787 | 56.091 | 32.994 | 1.00 | 17.54 | A |
| ATOM | 2717 | OD1 | ASN | A | 356 | 29.078 | 55.830 | 33.970 | 1.00 | 17.65 | A |
| ATOM | 2718 | ND2 | ASN | A | 356 | 30.335 | 57.288 | 32.804 | 1.00 | 15.84 | A |
| ATOM | 2719 | C | ASN | A | 356 | 30.803 | 52.630 | 31.696 | 1.00 | 16.96 | A |
| ATOM | 2720 | O | ASN | A | 356 | 30.013 | 51.749 | 32.035 | 1.00 | 16.52 | A |
| ATOM | 2721 | N | TYR | A | 357 | 31.572 | 52.519 | 30.616 | 1.00 | 16.22 | A |
| ATOM | 2722 | CA | TYR | A | 357 | 31.492 | 51.316 | 29.799 | 1.00 | 16.78 | A |
| ATOM | 2723 | CB | TYR | A | 357 | 32.144 | 51.539 | 28.427 | 1.00 | 15.91 | A |
| ATOM | 2724 | CG | TYR | A | 357 | 31.149 | 52.056 | 27.414 | 1.00 | 14.97 | A |
| ATOM | 2725 | CD1 | TYR | A | 357 | 30.957 | 53.424 | 27.222 | 1.00 | 14.35 | A |
| ATOM | 2726 | CE1 | TYR | A | 357 | 29.976 | 53.900 | 26.353 | 1.00 | 14.39 | A |
| ATOM | 2727 | CD2 | TYR | A | 357 | 30.334 | 51.172 | 26.707 | 1.00 | 15.07 | A |
| ATOM | 2728 | CE2 | TYR | A | 357 | 29.347 | 51.634 | 25.842 | 1.00 | 14.68 | A |
| ATOM | 2729 | CZ | TYR | A | 357 | 29.172 | 52.998 | 25.670 | 1.00 | 14.63 | A |
| ATOM | 2730 | OH | TYR | A | 357 | 28.185 | 53.451 | 24.826 | 1.00 | 14.20 | A |
| ATOM | 2731 | C | TYR | A | 357 | 32.098 | 50.107 | 30.507 | 1.00 | 16.81 | A |
| ATOM | 2732 | O | TYR | A | 357 | 31.640 | 48.979 | 30.320 | 1.00 | 17.35 | A |
| ATOM | 2733 | N | GLU | A | 358 | 33.118 | 50.331 | 31.328 | 1.00 | 17.12 | A |
| ATOM | 2734 | CA | GLU | A | 358 | 33.720 | 49.223 | 32.067 | 1.00 | 17.90 | A |
| ATOM | 2735 | CB | GLU | A | 358 | 34.941 | 49.692 | 32.860 | 1.00 | 18.33 | A |
| ATOM | 2736 | CG | GLU | A | 358 | 36.183 | 49.960 | 32.018 | 1.00 | 20.36 | A |
| ATOM | 2737 | CD | GLU | A | 358 | 37.376 | 50.366 | 32.867 | 1.00 | 21.30 | A |
| ATOM | 2738 | OE1 | GLU | A | 358 | 37.230 | 50.442 | 34.106 | 1.00 | 23.37 | A |
| ATOM | 2739 | OE2 | GLU | A | 358 | 38.462 | 50.610 | 32.302 | 1.00 | 22.02 | A |
| ATOM | 2740 | C | GLU | A | 358 | 32.677 | 48.647 | 33.032 | 1.00 | 18.18 | A |
| ATOM | 2741 | O | GLU | A | 358 | 32.622 | 47.436 | 33.251 | 1.00 | 18.62 | A |
| ATOM | 2742 | N | ARG | A | 359 | 31.853 | 49.516 | 33.610 | 1.00 | 18.25 | A |
| ATOM | 2743 | CA | ARG | A | 359 | 30.816 | 49.067 | 34.540 | 1.00 | 19.06 | A |
| ATOM | 2744 | CB | ARG | A | 359 | 30.148 | 50.262 | 35.217 | 1.00 | 20.23 | A |
| ATOM | 2745 | CG | ARG | A | 359 | 31.040 | 50.962 | 36.213 | 1.00 | 23.21 | A |
| ATOM | 2746 | CD | ARG | A | 359 | 30.417 | 52.260 | 36.691 | 1.00 | 25.64 | A |
| ATOM | 2747 | NE | ARG | A | 359 | 31.326 | 52.987 | 37.572 | 1.00 | 27.54 | A |
| ATOM | 2748 | CZ | ARG | A | 359 | 31.389 | 54.312 | 37.640 | 1.00 | 28.87 | A |
| ATOM | 2749 | NH1 | ARG | A | 359 | 30.592 | 55.052 | 36.876 | 1.00 | 28.85 | A |
| ATOM | 2750 | NH2 | ARG | A | 359 | 32.252 | 54.896 | 38.464 | 1.00 | 29.94 | A |
| ATOM | 2751 | C | ARG | A | 359 | 29.768 | 48.239 | 33.814 | 1.00 | 18.37 | A |
| ATOM | 2752 | O | ARG | A | 359 | 29.294 | 47.224 | 34.333 | 1.00 | 16.91 | A |
| ATOM | 2753 | N | LEU | A | 360 | 29.407 | 48.675 | 32.611 | 1.00 | 17.62 | A |
| ATOM | 2754 | CA | LEU | A | 360 | 28.423 | 47.960 | 31.811 | 1.00 | 18.02 | A |
| ATOM | 2755 | CB | LEU | A | 360 | 28.047 | 48.782 | 30.576 | 1.00 | 17.79 | A |
| ATOM | 2756 | CG | LEU | A | 360 | 27.214 | 50.040 | 30.849 | 1.00 | 17.79 | A |
| ATOM | 2757 | CD1 | LEU | A | 360 | 27.164 | 50.912 | 29.602 | 1.00 | 17.80 | A |
| ATOM | 2758 | CD2 | LEU | A | 360 | 25.807 | 49.639 | 31.282 | 1.00 | 17.86 | A |
| ATOM | 2759 | C | LEU | A | 360 | 28.950 | 46.590 | 31.392 | 1.00 | 18.31 | A |
| ATOM | 2760 | O | LEU | A | 360 | 28.222 | 45.599 | 31.446 | 1.00 | 18.36 | A |
| ATOM | 2761 | N | PHE | A | 361 | 30.214 | 46.536 | 30.977 | 1.00 | 17.86 | A |
| ATOM | 2762 | CA | PHE | A | 361 | 30.831 | 45.277 | 30.562 | 1.00 | 18.13 | A |
| ATOM | 2763 | CB | PHE | A | 361 | 32.253 | 45.518 | 30.038 | 1.00 | 17.21 | A |
| ATOM | 2764 | CG | PHE | A | 361 | 32.313 | 46.324 | 28.768 | 1.00 | 16.77 | A |
| ATOM | 2765 | CD1 | PHE | A | 361 | 33.507 | 46.927 | 28.376 | 1.00 | 17.09 | A |
| ATOM | 2766 | CD2 | PHE | A | 361 | 31.188 | 46.481 | 27.967 | 1.00 | 16.11 | A |
| ATOM | 2767 | CE1 | PHE | A | 361 | 33.577 | 47.676 | 27.204 | 1.00 | 15.66 | A |
| ATOM | 2768 | CE2 | PHE | A | 361 | 31.247 | 47.229 | 26.790 | 1.00 | 16.59 | A |
| ATOM | 2769 | CZ | PHE | A | 361 | 32.442 | 47.826 | 26.410 | 1.00 | 17.03 | A |
| ATOM | 2770 | C | PHE | A | 361 | 30.900 | 44.265 | 31.708 | 1.00 | 18.81 | A |
| ATOM | 2771 | O | PHE | A | 361 | 30.568 | 43.091 | 31.528 | 1.00 | 17.83 | A |
| ATOM | 2772 | N | GLU | A | 362 | 31.344 | 44.711 | 32.881 | 1.00 | 19.34 | A |
| ATOM | 2773 | CA | GLU | A | 362 | 31.455 | 43.798 | 34.016 | 1.00 | 20.55 | A |
| ATOM | 2774 | CB | GLU | A | 362 | 31.992 | 44.516 | 35.258 | 1.00 | 22.52 | A |
| ATOM | 2775 | CG | GLU | A | 362 | 32.331 | 43.551 | 36.397 | 1.00 | 25.60 | A |
| ATOM | 2776 | CD | GLU | A | 362 | 32.723 | 44.256 | 37.683 | 1.00 | 27.36 | A |
| ATOM | 2777 | OE1 | GLU | A | 362 | 33.457 | 45.262 | 37.608 | 1.00 | 29.24 | A |
| ATOM | 2778 | OE2 | GLU | A | 362 | 32.309 | 43.796 | 38.770 | 1.00 | 29.36 | A |
| ATOM | 2779 | C | GLU | A | 362 | 30.105 | 43.171 | 34.343 | 1.00 | 20.30 | A |
| ATOM | 2780 | O | GLU | A | 362 | 30.010 | 41.963 | 34.560 | 1.00 | 20.30 | A |
| ATOM | 2781 | N | HIS | A | 363 | 29.060 | 43.990 | 34.373 | 1.00 | 20.05 | A |
| ATOM | 2782 | CA | HIS | A | 363 | 27.730 | 43.485 | 34.676 | 1.00 | 20.56 | A |
| ATOM | 2783 | CB | HIS | A | 363 | 26.734 | 44.636 | 34.814 | 1.00 | 22.06 | A |
| ATOM | 2784 | CG | HIS | A | 363 | 25.352 | 44.191 | 35.182 | 1.00 | 23.46 | A |
| ATOM | 2785 | CD2 | HIS | A | 363 | 24.204 | 44.156 | 34.463 | 1.00 | 24.15 | A |
| ATOM | 2786 | ND1 | HIS | A | 363 | 25.043 | 43.673 | 36.421 | 1.00 | 24.49 | A |
| ATOM | 2787 | CE1 | HIS | A | 363 | 23.765 | 43.338 | 36.450 | 1.00 | 24.15 | A |
| ATOM | 2788 | NE2 | HIS | A | 363 | 23.233 | 43.620 | 35.274 | 1.00 | 24.25 | A |
| ATOM | 2789 | C | HIS | A | 363 | 27.233 | 42.531 | 33.596 | 1.00 | 19.95 | A |
| ATOM | 2790 | O | HIS | A | 363 | 26.919 | 41.371 | 33.868 | 1.00 | 19.76 | A |
| ATOM | 2791 | N | ILE | A | 364 | 27.164 | 43.029 | 32.368 | 1.00 | 19.34 | A |
| ATOM | 2792 | CA | ILE | A | 364 | 26.681 | 42.237 | 31.248 | 1.00 | 19.40 | A |
| ATOM | 2793 | CB | ILE | A | 364 | 26.783 | 43.031 | 29.929 | 1.00 | 18.87 | A |
| ATOM | 2794 | CG2 | ILE | A | 364 | 26.359 | 42.149 | 28.751 | 1.00 | 18.65 | A |
| ATOM | 2795 | CG1 | ILE | A | 364 | 25.894 | 44.277 | 30.007 | 1.00 | 19.23 | A |
| ATOM | 2796 | CD1 | ILE | A | 364 | 26.025 | 45.203 | 28.816 | 1.00 | 19.57 | A |
| ATOM | 2797 | C | ILE | A | 364 | 27.400 | 40.902 | 31.079 | 1.00 | 19.15 | A |
| ATOM | 2798 | O | ILE | A | 364 | 26.755 | 39.863 | 30.931 | 1.00 | 19.49 | A |
| ATOM | 2799 | N | ASN | A | 365 | 28.728 | 40.921 | 31.112 | 1.00 | 18.92 | A |
| ATOM | 2800 | CA | ASN | A | 365 | 29.491 | 39.691 | 30.934 | 1.00 | 20.01 | A |
| ATOM | 2801 | CB | ASN | A | 365 | 30.970 | 40.010 | 30.701 | 1.00 | 18.96 | A |
| ATOM | 2802 | CG | ASN | A | 365 | 31.189 | 40.886 | 29.481 | 1.00 | 18.43 | A |
| ATOM | 2803 | OD1 | ASN | A | 365 | 30.294 | 41.044 | 28.652 | 1.00 | 18.38 | A |
| ATOM | 2804 | ND2 | ASN | A | 365 | 32.385 | 41.452 | 29.362 | 1.00 | 17.32 | A |
| ATOM | 2805 | C | ASN | A | 365 | 29.356 | 38.697 | 32.088 | 1.00 | 21.35 | A |
| ATOM | 2806 | O | ASN | A | 365 | 29.670 | 37.519 | 31.928 | 1.00 | 21.12 | A |
| ATOM | 2807 | N | SER | A | 366 | 28.880 | 39.165 | 33.239 | 1.00 | 22.96 | A |
| ATOM | 2808 | CA | SER | A | 366 | 28.714 | 38.291 | 34.401 | 1.00 | 24.46 | A |
| ATOM | 2809 | CB | SER | A | 366 | 29.198 | 38.999 | 35.674 | 1.00 | 24.61 | A |
| ATOM | 2810 | OG | SER | A | 366 | 28.384 | 40.117 | 35.978 | 1.00 | 24.98 | A |
| ATOM | 2811 | C | SER | A | 366 | 27.264 | 37.843 | 34.577 | 1.00 | 25.44 | A |
| ATOM | 2812 | O | SER | A | 366 | 26.956 | 37.046 | 35.460 | 1.00 | 25.76 | A |
| ATOM | 2813 | N | GLN | A | 367 | 26.375 | 38.364 | 33.736 | 1.00 | 26.52 | A |
| ATOM | 2814 | CA | GLN | A | 367 | 24.961 | 38.003 | 33.779 | 1.00 | 27.62 | A |
| ATOM | 2815 | CB | GLN | A | 367 | 24.083 | 39.251 | 33.640 | 1.00 | 28.64 | A |
| ATOM | 2816 | CG | GLN | A | 367 | 24.113 | 40.174 | 34.850 | 1.00 | 30.61 | A |
| ATOM | 2817 | CD | GLN | A | 367 | 23.525 | 39.520 | 36.088 | 1.00 | 32.01 | A |
| ATOM | 2818 | OE1 | GLN | A | 367 | 22.341 | 39.176 | 36.119 | 1.00 | 32.79 | A |
| ATOM | 2819 | NE2 | GLN | A | 367 | 24.352 | 39.340 | 37.113 | 1.00 | 32.51 | A |
| ATOM | 2820 | C | GLN | A | 367 | 24.665 | 37.033 | 32.636 | 1.00 | 27.49 | A |
| ATOM | 2821 | O | GLN | A | 367 | 24.335 | 37.447 | 31.524 | 1.00 | 27.23 | A |
| ATOM | 2822 | N | ALA | A | 368 | 24.778 | 35.740 | 32.926 | 1.00 | 27.43 | A |
| ATOM | 2823 | CA | ALA | A | 368 | 24.551 | 34.689 | 31.938 | 1.00 | 27.09 | A |
| ATOM | 2824 | CB | ALA | A | 368 | 24.531 | 33.327 | 32.633 | 1.00 | 27.33 | A |
| ATOM | 2825 | C | ALA | A | 368 | 23.291 | 34.851 | 31.087 | 1.00 | 26.68 | A |
| ATOM | 2826 | O | ALA | A | 368 | 23.311 | 34.568 | 29.889 | 1.00 | 26.56 | A |
| ATOM | 2827 | N | HIS | A | 369 | 22.199 | 35.303 | 31.697 | 1.00 | 26.31 | A |
| ATOM | 2828 | CA | HIS | A | 369 | 20.942 | 35.464 | 30.970 | 1.00 | 25.66 | A |
| ATOM | 2829 | CB | HIS | A | 369 | 19.852 | 35.981 | 31.916 | 1.00 | 27.03 | A |
| ATOM | 2830 | CG | HIS | A | 369 | 20.044 | 37.401 | 32.346 | 1.00 | 27.87 | A |
| ATOM | 2831 | CD2 | HIS | A | 369 | 20.642 | 37.928 | 33.441 | 1.00 | 28.21 | A |
| ATOM | 2832 | ND1 | HIS | A | 369 | 19.610 | 38.471 | 31.594 | 1.00 | 28.33 | A |
| ATOM | 2833 | CE1 | HIS | A | 369 | 19.932 | 39.597 | 32.207 | 1.00 | 28.26 | A |
| ATOM | 2834 | NE2 | HIS | A | 369 | 20.559 | 39.295 | 33.330 | 1.00 | 28.33 | A |
| ATOM | 2835 | C | HIS | A | 369 | 21.059 | 36.375 | 29.744 | 1.00 | 25.04 | A |
| ATOM | 2836 | O | HIS | A | 369 | 20.210 | 36.340 | 28.853 | 1.00 | 24.82 | A |
| ATOM | 2837 | N | PHE | A | 370 | 22.109 | 37.189 | 29.697 | 1.00 | 24.10 | A |
| ATOM | 2838 | CA | PHE | A | 370 | 22.320 | 38.080 | 28.559 | 1.00 | 22.85 | A |
| ATOM | 2839 | CB | PHE | A | 370 | 23.235 | 39.247 | 28.944 | 1.00 | 23.57 | A |
| ATOM | 2840 | CG | PHE | A | 370 | 22.533 | 40.365 | 29.669 | 1.00 | 23.73 | A |
| ATOM | 2841 | CD1 | PHE | A | 370 | 23.086 | 40.915 | 30.819 | 1.00 | 24.30 | A |
| ATOM | 2842 | CD2 | PHE | A | 370 | 21.338 | 40.890 | 29.184 | 1.00 | 24.44 | A |
| ATOM | 2843 | CE1 | PHE | A | 370 | 22.460 | 41.973 | 31.480 | 1.00 | 24.01 | A |
| ATOM | 2844 | CE2 | PHE | A | 370 | 20.704 | 41.950 | 29.839 | 1.00 | 24.36 | A |
| ATOM | 2845 | CZ | PHE | A | 370 | 21.270 | 42.490 | 30.989 | 1.00 | 23.88 | A |
| ATOM | 2846 | C | PHE | A | 370 | 22.964 | 37.308 | 27.414 | 1.00 | 21.51 | A |
| ATOM | 2847 | O | PHE | A | 370 | 22.698 | 37.585 | 26.244 | 1.00 | 20.83 | A |
| ATOM | 2848 | N | ASN | A | 371 | 23.806 | 36.339 | 27.768 | 1.00 | 19.82 | A |
| ATOM | 2849 | CA | ASN | A | 371 | 24.521 | 35.528 | 26.789 | 1.00 | 19.02 | A |
| ATOM | 2850 | CB | ASN | A | 371 | 23.538 | 34.661 | 26.007 | 1.00 | 19.65 | A |
| ATOM | 2851 | CG | ASN | A | 371 | 22.892 | 33.599 | 26.877 | 1.00 | 20.03 | A |
| ATOM | 2852 | OD1 | ASN | A | 371 | 23.573 | 32.718 | 27.405 | 1.00 | 20.28 | A |
| ATOM | 2853 | ND2 | ASN | A | 371 | 21.578 | 33.682 | 27.038 | 1.00 | 20.15 | A |
| ATOM | 2854 | C | ASN | A | 371 | 25.316 | 36.430 | 25.850 | 1.00 | 18.66 | A |
| ATOM | 2855 | O | ASN | A | 371 | 25.340 | 36.229 | 24.633 | 1.00 | 17.96 | A |
| ATOM | 2856 | N | VAL | A | 372 | 25.969 | 37.424 | 26.447 | 1.00 | 18.22 | A |
| ATOM | 2857 | CA | VAL | A | 372 | 26.777 | 38.395 | 25.721 | 1.00 | 18.11 | A |
| ATOM | 2858 | CB | VAL | A | 372 | 26.094 | 39.788 | 25.701 | 1.00 | 18.45 | A |
| ATOM | 2859 | CG1 | VAL | A | 372 | 27.065 | 40.851 | 25.163 | 1.00 | 17.90 | A |
| ATOM | 2860 | CG2 | VAL | A | 372 | 24.834 | 39.739 | 24.855 | 1.00 | 18.52 | A |
| ATOM | 2861 | C | VAL | A | 372 | 28.146 | 38.564 | 26.372 | 1.00 | 18.05 | A |
| ATOM | 2862 | O | VAL | A | 372 | 28.274 | 38.520 | 27.594 | 1.00 | 17.93 | A |
| ATOM | 2863 | N | GLN | A | 373 | 29.162 | 38.751 | 25.538 | 1.00 | 17.52 | A |
| ATOM | 2864 | CA | GLN | A | 373 | 30.528 | 38.982 | 25.995 | 1.00 | 17.49 | A |
| ATOM | 2865 | CB | GLN | A | 373 | 31.442 | 37.823 | 25.579 | 1.00 | 18.60 | A |
| ATOM | 2866 | CG | GLN | A | 373 | 32.923 | 38.011 | 25.922 | 1.00 | 19.99 | A |
| ATOM | 2867 | CD | GLN | A | 373 | 33.158 | 38.334 | 27.394 | 1.00 | 20.94 | A |
| ATOM | 2868 | OE1 | GLN | A | 373 | 32.526 | 37.756 | 28.279 | 1.00 | 21.70 | A |
| ATOM | 2869 | NE2 | GLN | A | 373 | 34.082 | 39.252 | 27.659 | 1.00 | 20.60 | A |
| ATOM | 2870 | C | GLN | A | 373 | 30.935 | 40.270 | 25.282 | 1.00 | 17.32 | A |
| ATOM | 2871 | O | GLN | A | 373 | 31.179 | 40.265 | 24.079 | 1.00 | 17.16 | A |
| ATOM | 2872 | N | ALA | A | 374 | 30.984 | 41.371 | 26.023 | 1.00 | 16.74 | A |
| ATOM | 2873 | CA | ALA | A | 374 | 31.325 | 42.665 | 25.445 | 1.00 | 16.51 | A |
| ATOM | 2874 | CB | ALA | A | 374 | 30.217 | 43.671 | 25.756 | 1.00 | 15.86 | A |
| ATOM | 2875 | C | ALA | A | 374 | 32.662 | 43.202 | 25.940 | 1.00 | 16.97 | A |
| ATOM | 2876 | O | ALA | A | 374 | 33.046 | 42.984 | 27.087 | 1.00 | 16.37 | A |
| ATOM | 2877 | N | GLN | A | 375 | 33.364 | 43.920 | 25.070 | 1.00 | 16.76 | A |
| ATOM | 2878 | CA | GLN | A | 375 | 34.650 | 44.498 | 25.440 | 1.00 | 17.48 | A |
| ATOM | 2879 | CB | GLN | A | 375 | 35.731 | 43.415 | 25.509 | 1.00 | 19.85 | A |
| ATOM | 2880 | CG | GLN | A | 375 | 35.933 | 42.644 | 24.205 | 1.00 | 23.52 | A |
| ATOM | 2881 | CD | GLN | A | 375 | 35.066 | 41.404 | 24.129 | 1.00 | 26.57 | A |
| ATOM | 2882 | OE1 | GLN | A | 375 | 35.214 | 40.486 | 24.939 | 1.00 | 27.84 | A |
| ATOM | 2883 | NE2 | GLN | A | 375 | 34.153 | 41.370 | 23.161 | 1.00 | 27.24 | A |
| ATOM | 2884 | C | GLN | A | 375 | 35.085 | 45.548 | 24.431 | 1.00 | 16.70 | A |
| ATOM | 2885 | O | GLN | A | 375 | 34.534 | 45.632 | 23.335 | 1.00 | 15.55 | A |
| ATOM | 2886 | N | PHE | A | 376 | 36.069 | 46.356 | 24.814 | 1.00 | 15.17 | A |
| ATOM | 2887 | CA | PHE | A | 376 | 36.602 | 47.358 | 23.904 | 1.00 | 14.46 | A |
| ATOM | 2888 | CB | PHE | A | 376 | 37.543 | 48.320 | 24.637 | 1.00 | 13.95 | A |
| ATOM | 2889 | CG | PHE | A | 376 | 36.848 | 49.228 | 25.610 | 1.00 | 14.70 | A |
| ATOM | 2890 | CD1 | PHE | A | 376 | 37.246 | 49.273 | 26.940 | 1.00 | 14.81 | A |
| ATOM | 2891 | CD2 | PHE | A | 376 | 35.798 | 50.044 | 25.195 | 1.00 | 14.57 | A |
| ATOM | 2892 | CE1 | PHE | A | 376 | 36.610 | 50.117 | 27.848 | 1.00 | 15.62 | A |
| ATOM | 2893 | CE2 | PHE | A | 376 | 35.157 | 50.894 | 26.098 | 1.00 | 15.64 | A |
| ATOM | 2894 | CZ | PHE | A | 376 | 35.567 | 50.928 | 27.428 | 1.00 | 14.70 | A |
| ATOM | 2895 | C | PHE | A | 376 | 37.396 | 46.567 | 22.877 | 1.00 | 13.69 | A |
| ATOM | 2896 | O | PHE | A | 376 | 38.028 | 45.566 | 23.214 | 1.00 | 13.20 | A |
| ATOM | 2897 | N | GLY | A | 377 | 37.360 | 47.005 | 21.625 | 1.00 | 13.56 | A |
| ATOM | 2898 | CA | GLY | A | 377 | 38.106 | 46.305 | 20.598 | 1.00 | 13.03 | A |
| ATOM | 2899 | C | GLY | A | 377 | 38.537 | 47.240 | 19.490 | 1.00 | 13.47 | A |
| ATOM | 2900 | O | GLY | A | 377 | 38.226 | 48.432 | 19.519 | 1.00 | 12.17 | A |
| ATOM | 2901 | N | THR | A | 378 | 39.270 | 46.704 | 18.522 | 1.00 | 13.13 | A |
| ATOM | 2902 | CA | THR | A | 378 | 39.712 | 47.492 | 17.384 | 1.00 | 14.10 | A |
| ATOM | 2903 | CB | THR | A | 378 | 41.226 | 47.348 | 17.129 | 1.00 | 14.13 | A |
| ATOM | 2904 | OG1 | THR | A | 378 | 41.521 | 46.003 | 16.739 | 1.00 | 15.02 | A |
| ATOM | 2905 | CG2 | THR | A | 378 | 42.012 | 47.697 | 18.385 | 1.00 | 15.10 | A |
| ATOM | 2906 | C | THR | A | 378 | 38.961 | 46.973 | 16.167 | 1.00 | 13.64 | A |
| ATOM | 2907 | O | THR | A | 378 | 38.278 | 45.945 | 16.238 | 1.00 | 13.55 | A |
| ATOM | 2908 | N | LEU | A | 379 | 39.084 | 47.684 | 15.054 | 1.00 | 12.92 | A |
| ATOM | 2909 | CA | LEU | A | 379 | 38.409 | 47.289 | 13.827 | 1.00 | 12.73 | A |
| ATOM | 2910 | CB | LEU | A | 379 | 38.649 | 48.342 | 12.742 | 1.00 | 12.44 | A |
| ATOM | 2911 | CG | LEU | A | 379 | 37.870 | 48.131 | 11.445 | 1.00 | 12.37 | A |
| ATOM | 2912 | CD1 | LEU | A | 379 | 36.372 | 48.184 | 11.741 | 1.00 | 11.04 | A |
| ATOM | 2913 | CD2 | LEU | A | 379 | 38.270 | 49.205 | 10.433 | 1.00 | 11.81 | A |
| ATOM | 2914 | C | LEU | A | 379 | 38.896 | 45.924 | 13.338 | 1.00 | 13.00 | A |
| ATOM | 2915 | O | LEU | A | 379 | 38.098 | 45.074 | 12.934 | 1.00 | 12.40 | A |
| ATOM | 2916 | N | GLN | A | 380 | 40.208 | 45.714 | 13.384 | 1.00 | 13.46 | A |
| ATOM | 2917 | CA | GLN | A | 380 | 40.782 | 44.450 | 12.936 | 1.00 | 14.77 | A |
| ATOM | 2918 | CB | GLN | A | 380 | 42.309 | 44.516 | 12.986 | 1.00 | 15.81 | A |
| ATOM | 2919 | CG | GLN | A | 380 | 42.995 | 43.306 | 12.376 | 1.00 | 19.57 | A |
| ATOM | 2920 | CD | GLN | A | 380 | 42.592 | 43.086 | 10.930 | 1.00 | 21.70 | A |
| ATOM | 2921 | OE1 | GLN | A | 380 | 42.669 | 44.001 | 10.106 | 1.00 | 22.56 | A |
| ATOM | 2922 | NE2 | GLN | A | 380 | 42.162 | 41.866 | 10.612 | 1.00 | 22.92 | A |
| ATOM | 2923 | C | GLN | A | 380 | 40.285 | 43.285 | 13.791 | 1.00 | 14.48 | A |
| ATOM | 2924 | O | GLN | A | 380 | 40.054 | 42.187 | 13.280 | 1.00 | 13.99 | A |
| ATOM | 2925 | N | GLU | A | 381 | 40.127 | 43.522 | 15.090 | 1.00 | 14.50 | A |
| ATOM | 2926 | CA | GLU | A | 381 | 39.653 | 42.474 | 15.985 | 1.00 | 15.49 | A |
| ATOM | 2927 | CB | GLU | A | 381 | 39.648 | 42.962 | 17.439 | 1.00 | 17.08 | A |
| ATOM | 2928 | CG | GLU | A | 381 | 41.038 | 43.325 | 17.957 | 1.00 | 21.28 | A |
| ATOM | 2929 | CD | GLU | A | 381 | 41.063 | 43.610 | 19.448 | 1.00 | 22.82 | A |
| ATOM | 2930 | OE1 | GLU | A | 381 | 40.241 | 44.415 | 19.921 | 1.00 | 22.89 | A |
| ATOM | 2931 | OE2 | GLU | A | 381 | 41.921 | 43.032 | 20.148 | 1.00 | 27.14 | A |
| ATOM | 2932 | C | GLU | A | 381 | 38.252 | 42.041 | 15.573 | 1.00 | 14.29 | A |
| ATOM | 2933 | O | GLU | A | 381 | 37.937 | 40.853 | 15.571 | 1.00 | 13.97 | A |
| ATOM | 2934 | N | TYR | A | 382 | 37.413 | 43.011 | 15.227 | 1.00 | 13.36 | A |
| ATOM | 2935 | CA | TYR | A | 382 | 36.058 | 42.709 | 14.796 | 1.00 | 13.03 | A |
| ATOM | 2936 | CB | TYR | A | 382 | 35.294 | 43.993 | 14.468 | 1.00 | 13.08 | A |
| ATOM | 2937 | CG | TYR | A | 382 | 33.985 | 43.722 | 13.763 | 1.00 | 13.27 | A |
| ATOM | 2938 | CD1 | TYR | A | 382 | 32.928 | 43.096 | 14.430 | 1.00 | 13.48 | A |
| ATOM | 2939 | CE1 | TYR | A | 382 | 31.746 | 42.766 | 13.764 | 1.00 | 11.84 | A |
| ATOM | 2940 | CD2 | TYR | A | 382 | 33.826 | 44.020 | 12.407 | 1.00 | 12.53 | A |
| ATOM | 2941 | CE2 | TYR | A | 382 | 32.650 | 43.693 | 11.732 | 1.00 | 12.76 | A |
| ATOM | 2942 | CZ | TYR | A | 382 | 31.615 | 43.062 | 12.419 | 1.00 | 12.40 | A |
| ATOM | 2943 | OH | TYR | A | 382 | 30.461 | 42.708 | 11.749 | 1.00 | 12.59 | A |
| ATOM | 2944 | C | TYR | A | 382 | 36.078 | 41.820 | 13.554 | 1.00 | 13.04 | A |
| ATOM | 2945 | O | TYR | A | 382 | 35.451 | 40.761 | 13.522 | 1.00 | 12.38 | A |
| ATOM | 2946 | N | PHE | A | 383 | 36.791 | 42.261 | 12.523 | 1.00 | 12.27 | A |
| ATOM | 2947 | CA | PHE | A | 383 | 36.864 | 41.496 | 11.285 | 1.00 | 13.24 | A |
| ATOM | 2948 | CB | PHE | A | 383 | 37.653 | 42.281 | 10.227 | 1.00 | 13.43 | A |
| ATOM | 2949 | CG | PHE | A | 383 | 36.876 | 43.417 | 9.605 | 1.00 | 12.96 | A |
| ATOM | 2950 | CD1 | PHE | A | 383 | 37.365 | 44.717 | 9.648 | 1.00 | 13.91 | A |
| ATOM | 2951 | CD2 | PHE | A | 383 | 35.659 | 43.181 | 8.968 | 1.00 | 14.11 | A |
| ATOM | 2952 | CE1 | PHE | A | 383 | 36.658 | 45.772 | 9.064 | 1.00 | 13.79 | A |
| ATOM | 2953 | CE2 | PHE | A | 383 | 34.939 | 44.224 | 8.379 | 1.00 | 14.72 | A |
| ATOM | 2954 | CZ | PHE | A | 383 | 35.441 | 45.525 | 8.426 | 1.00 | 14.80 | A |
| ATOM | 2955 | C | PHE | A | 383 | 37.460 | 40.095 | 11.474 | 1.00 | 14.00 | A |
| ATOM | 2956 | O | PHE | A | 383 | 36.984 | 39.129 | 10.873 | 1.00 | 13.60 | A |
| ATOM | 2957 | N | ASP | A | 384 | 38.494 | 39.976 | 12.303 | 1.00 | 14.72 | A |
| ATOM | 2958 | CA | ASP | A | 384 | 39.098 | 38.662 | 12.540 | 1.00 | 15.43 | A |
| ATOM | 2959 | CB | ASP | A | 384 | 40.272 | 38.759 | 13.517 | 1.00 | 16.51 | A |
| ATOM | 2960 | CG | ASP | A | 384 | 41.505 | 39.384 | 12.898 | 1.00 | 17.75 | A |
| ATOM | 2961 | OD1 | ASP | A | 384 | 41.584 | 39.464 | 11.652 | 1.00 | 18.23 | A |
| ATOM | 2962 | OD2 | ASP | A | 384 | 42.405 | 39.782 | 13.666 | 1.00 | 19.50 | A |
| ATOM | 2963 | C | ASP | A | 384 | 38.054 | 37.710 | 13.116 | 1.00 | 15.26 | A |
| ATOM | 2964 | O | ASP | A | 384 | 37.960 | 36.553 | 12.704 | 1.00 | 15.46 | A |
| ATOM | 2965 | N | ALA | A | 385 | 37.265 | 38.206 | 14.064 | 1.00 | 14.92 | A |
| ATOM | 2966 | CA | ALA | A | 385 | 36.226 | 37.398 | 14.696 | 1.00 | 15.13 | A |
| ATOM | 2967 | CB | ALA | A | 385 | 35.606 | 38.161 | 15.866 | 1.00 | 14.89 | A |
| ATOM | 2968 | C | ALA | A | 385 | 35.149 | 37.013 | 13.688 | 1.00 | 15.31 | A |
| ATOM | 2969 | O | ALA | A | 385 | 34.693 | 35.868 | 13.657 | 1.00 | 15.33 | A |
| ATOM | 2970 | N | VAL | A | 386 | 34.737 | 37.969 | 12.863 | 1.00 | 15.18 | A |
| ATOM | 2971 | CA | VAL | A | 386 | 33.722 | 37.698 | 11.851 | 1.00 | 15.40 | A |
| ATOM | 2972 | CB | VAL | A | 386 | 33.453 | 38.941 | 10.980 | 1.00 | 15.05 | A |
| ATOM | 2973 | CG1 | VAL | A | 386 | 32.561 | 38.567 | 9.793 | 1.00 | 15.44 | A |
| ATOM | 2974 | CG2 | VAL | A | 386 | 32.788 | 40.022 | 11.819 | 1.00 | 15.00 | A |
| ATOM | 2975 | C | VAL | A | 386 | 34.153 | 36.548 | 10.938 | 1.00 | 16.11 | A |
| ATOM | 2976 | O | VAL | A | 386 | 33.387 | 35.610 | 10.690 | 1.00 | 16.01 | A |
| ATOM | 2977 | N | HIS | A | 387 | 35.382 | 36.613 | 10.443 | 1.00 | 16.13 | A |
| ATOM | 2978 | CA | HIS | A | 387 | 35.869 | 35.570 | 9.557 | 1.00 | 16.83 | A |
| ATOM | 2979 | CB | HIS | A | 387 | 37.129 | 36.053 | 8.840 | 1.00 | 16.93 | A |
| ATOM | 2980 | CG | HIS | A | 387 | 36.860 | 37.172 | 7.881 | 1.00 | 17.54 | A |
| ATOM | 2981 | CD2 | HIS | A | 387 | 37.271 | 38.462 | 7.874 | 1.00 | 17.35 | A |
| ATOM | 2982 | ND1 | HIS | A | 387 | 36.022 | 37.030 | 6.796 | 1.00 | 17.71 | A |
| ATOM | 2983 | CE1 | HIS | A | 387 | 35.926 | 38.186 | 6.163 | 1.00 | 17.77 | A |
| ATOM | 2984 | NE2 | HIS | A | 387 | 36.674 | 39.072 | 6.798 | 1.00 | 17.27 | A |
| ATOM | 2985 | C | HIS | A | 387 | 36.090 | 34.243 | 10.274 | 1.00 | 17.78 | A |
| ATOM | 2986 | O | HIS | A | 387 | 36.055 | 33.181 | 9.651 | 1.00 | 16.94 | A |
| ATOM | 2987 | N | GLN | A | 388 | 36.307 | 34.300 | 11.583 | 1.00 | 19.02 | A |
| ATOM | 2988 | CA | GLN | A | 388 | 36.474 | 33.077 | 12.358 | 1.00 | 21.11 | A |
| ATOM | 2989 | CB | GLN | A | 388 | 36.943 | 33.402 | 13.780 | 1.00 | 23.11 | A |
| ATOM | 2990 | CG | GLN | A | 388 | 38.439 | 33.692 | 13.900 | 1.00 | 25.90 | A |
| ATOM | 2991 | CD | GLN | A | 388 | 38.804 | 34.341 | 15.230 | 1.00 | 28.63 | A |
| ATOM | 2992 | OE1 | GLN | A | 388 | 38.246 | 34.000 | 16.275 | 1.00 | 30.49 | A |
| ATOM | 2993 | NE2 | GLN | A | 388 | 39.754 | 35.274 | 15.197 | 1.00 | 29.72 | A |
| ATOM | 2994 | C | GLN | A | 388 | 35.105 | 32.394 | 12.392 | 1.00 | 21.50 | A |
| ATOM | 2995 | O | GLN | A | 388 | 35.005 | 31.169 | 12.304 | 1.00 | 21.80 | A |
| ATOM | 2996 | N | ALA | A | 389 | 34.050 | 33.196 | 12.506 | 1.00 | 21.77 | A |
| ATOM | 2997 | CA | ALA | A | 389 | 32.686 | 32.674 | 12.535 | 1.00 | 22.68 | A |
| ATOM | 2998 | CB | ALA | A | 389 | 31.707 | 33.781 | 12.912 | 1.00 | 22.05 | A |
| ATOM | 2999 | C | ALA | A | 389 | 32.341 | 32.109 | 11.161 | 1.00 | 23.68 | A |
| ATOM | 3000 | O | ALA | A | 389 | 31.684 | 31.069 | 11.045 | 1.00 | 23.65 | A |
| ATOM | 3001 | N | GLU | A | 390 | 32.791 | 32.808 | 10.124 | 1.00 | 24.67 | A |
| ATOM | 3002 | CA | GLU | A | 390 | 32.564 | 32.406 | 8.741 | 1.00 | 26.15 | A |
| ATOM | 3003 | CB | GLU | A | 390 | 33.169 | 33.453 | 7.796 | 1.00 | 26.00 | A |
| ATOM | 3004 | CG | GLU | A | 390 | 33.252 | 33.042 | 6.328 | 1.00 | 26.38 | A |
| ATOM | 3005 | CD | GLU | A | 390 | 33.855 | 34.137 | 5.456 | 1.00 | 26.52 | A |
| ATOM | 3006 | OE1 | GLU | A | 390 | 34.755 | 34.851 | 5.944 | 1.00 | 25.91 | A |
| ATOM | 3007 | OE2 | GLU | A | 390 | 33.441 | 34.276 | 4.285 | 1.00 | 26.51 | A |
| ATOM | 3008 | C | GLU | A | 390 | 33.195 | 31.042 | 8.483 | 1.00 | 27.33 | A |
| ATOM | 3009 | O | GLU | A | 390 | 32.571 | 30.157 | 7.895 | 1.00 | 27.52 | A |
| ATOM | 3010 | N | ARG | A | 391 | 34.438 | 30.880 | 8.926 | 1.00 | 28.78 | A |
| ATOM | 3011 | CA | ARG | A | 391 | 35.157 | 29.626 | 8.751 | 1.00 | 30.39 | A |
| ATOM | 3012 | CB | ARG | A | 391 | 36.623 | 29.794 | 9.160 | 1.00 | 31.73 | A |
| ATOM | 3013 | CG | ARG | A | 391 | 37.466 | 30.566 | 8.153 | 1.00 | 33.97 | A |
| ATOM | 3014 | CD | ARG | A | 391 | 38.899 | 30.722 | 8.640 | 1.00 | 35.57 | A |
| ATOM | 3015 | NE | ARG | A | 391 | 38.987 | 31.640 | 9.770 | 1.00 | 38.15 | A |
| ATOM | 3016 | CZ | ARG | A | 391 | 40.082 | 31.831 | 10.499 | 1.00 | 39.05 | A |
| ATOM | 3017 | NH1 | ARG | A | 391 | 41.195 | 31.163 | 10.221 | 1.00 | 40.21 | A |
| ATOM | 3018 | NH2 | ARG | A | 391 | 40.065 | 32.693 | 11.506 | 1.00 | 39.40 | A |
| ATOM | 3019 | C | ARG | A | 391 | 34.516 | 28.505 | 9.564 | 1.00 | 30.59 | A |
| ATOM | 3020 | O | ARG | A | 391 | 34.605 | 27.333 | 9.198 | 1.00 | 31.38 | A |
| ATOM | 3021 | N | ALA | A | 392 | 33.874 | 28.867 | 10.669 | 1.00 | 30.51 | A |
| ATOM | 3022 | CA | ALA | A | 392 | 33.212 | 27.882 | 11.516 | 1.00 | 30.61 | A |
| ATOM | 3023 | CB | ALA | A | 392 | 32.878 | 28.494 | 12.873 | 1.00 | 30.84 | A |
| ATOM | 3024 | C | ALA | A | 392 | 31.939 | 27.415 | 10.819 | 1.00 | 30.66 | A |
| ATOM | 3025 | O | ALA | A | 392 | 31.261 | 26.495 | 11.283 | 1.00 | 30.52 | A |
| ATOM | 3026 | N | GLY | A | 393 | 31.621 | 28.065 | 9.703 | 1.00 | 30.35 | A |
| ATOM | 3027 | CA | GLY | A | 393 | 30.442 | 27.708 | 8.939 | 1.00 | 30.05 | A |
| ATOM | 3028 | C | GLY | A | 393 | 29.130 | 28.249 | 9.475 | 1.00 | 29.86 | A |
| ATOM | 3029 | O | GLY | A | 393 | 28.073 | 27.679 | 9.208 | 1.00 | 29.53 | A |
| ATOM | 3030 | N | GLN | A | 394 | 29.175 | 29.346 | 10.223 | 1.00 | 29.75 | A |
| ATOM | 3031 | CA | GLN | A | 394 | 27.939 | 29.902 | 10.755 | 1.00 | 29.86 | A |
| ATOM | 3032 | CB | GLN | A | 394 | 28.138 | 30.415 | 12.188 | 1.00 | 30.87 | A |
| ATOM | 3033 | CG | GLN | A | 394 | 28.875 | 31.732 | 12.311 | 1.00 | 31.79 | A |
| ATOM | 3034 | CD | GLN | A | 394 | 28.756 | 32.326 | 13.706 | 1.00 | 32.39 | A |
| ATOM | 3035 | OE1 | GLN | A | 394 | 29.228 | 31.747 | 14.687 | 1.00 | 32.51 | A |
| ATOM | 3036 | NE2 | GLN | A | 394 | 28.113 | 33.485 | 13.800 | 1.00 | 31.11 | A |
| ATOM | 3037 | C | GLN | A | 394 | 27.375 | 31.015 | 9.878 | 1.00 | 29.23 | A |
| ATOM | 3038 | O | GLN | A | 394 | 26.319 | 31.567 | 10.178 | 1.00 | 29.43 | A |
| ATOM | 3039 | N | ALA | A | 395 | 28.067 | 31.336 | 8.789 | 1.00 | 28.59 | A |
| ATOM | 3040 | CA | ALA | A | 395 | 27.596 | 32.385 | 7.892 | 1.00 | 27.95 | A |
| ATOM | 3041 | CB | ALA | A | 395 | 27.824 | 33.753 | 8.533 | 1.00 | 28.94 | A |
| ATOM | 3042 | C | ALA | A | 395 | 28.244 | 32.349 | 6.511 | 1.00 | 27.67 | A |
| ATOM | 3043 | O | ALA | A | 395 | 29.419 | 32.013 | 6.367 | 1.00 | 27.22 | A |
| ATOM | 3044 | N | GLU | A | 396 | 27.454 | 32.694 | 5.500 | 1.00 | 27.14 | A |
| ATOM | 3045 | CA | GLU | A | 396 | 27.917 | 32.745 | 4.118 | 1.00 | 27.12 | A |
| ATOM | 3046 | CB | GLU | A | 396 | 27.131 | 31.760 | 3.245 | 1.00 | 29.69 | A |
| ATOM | 3047 | CG | GLU | A | 396 | 26.527 | 30.582 | 4.002 | 1.00 | 34.14 | A |
| ATOM | 3048 | CD | GLU | A | 396 | 25.165 | 30.906 | 4.604 | 1.00 | 36.53 | A |
| ATOM | 3049 | OE1 | GLU | A | 396 | 25.068 | 31.857 | 5.413 | 1.00 | 38.43 | A |
| ATOM | 3050 | OE2 | GLU | A | 396 | 24.187 | 30.204 | 4.263 | 1.00 | 38.46 | A |
| ATOM | 3051 | C | GLU | A | 396 | 27.613 | 34.173 | 3.685 | 1.00 | 25.18 | A |
| ATOM | 3052 | O | GLU | A | 396 | 26.524 | 34.680 | 3.958 | 1.00 | 25.88 | A |
| ATOM | 3053 | N | PHE | A | 397 | 28.557 | 34.830 | 3.023 | 1.00 | 22.14 | A |
| ATOM | 3054 | CA | PHE | A | 397 | 28.328 | 36.211 | 2.619 | 1.00 | 19.29 | A |
| ATOM | 3055 | CB | PHE | A | 397 | 29.530 | 37.079 | 2.998 | 1.00 | 18.47 | A |
| ATOM | 3056 | CG | PHE | A | 397 | 29.776 | 37.147 | 4.476 | 1.00 | 17.12 | A |
| ATOM | 3057 | CD1 | PHE | A | 397 | 30.814 | 36.426 | 5.056 | 1.00 | 17.39 | A |
| ATOM | 3058 | CD2 | PHE | A | 397 | 28.948 | 37.909 | 5.293 | 1.00 | 16.43 | A |
| ATOM | 3059 | CE1 | PHE | A | 397 | 31.025 | 36.462 | 6.438 | 1.00 | 15.91 | A |
| ATOM | 3060 | CE2 | PHE | A | 397 | 29.148 | 37.953 | 6.673 | 1.00 | 16.34 | A |
| ATOM | 3061 | CZ | PHE | A | 397 | 30.190 | 37.227 | 7.245 | 1.00 | 15.91 | A |
| ATOM | 3062 | C | PHE | A | 397 | 27.999 | 36.403 | 1.148 | 1.00 | 18.17 | A |
| ATOM | 3063 | O | PHE | A | 397 | 28.569 | 35.749 | 0.278 | 1.00 | 17.72 | A |
| ATOM | 3064 | N | PRO | A | 398 | 27.072 | 37.327 | 0.857 | 1.00 | 16.93 | A |
| ATOM | 3065 | CD | PRO | A | 398 | 26.367 | 38.194 | 1.819 | 1.00 | 17.16 | A |
| ATOM | 3066 | CA | PRO | A | 398 | 26.653 | 37.622 | −0.512 | 1.00 | 16.50 | A |
| ATOM | 3067 | CB | PRO | A | 398 | 25.390 | 38.446 | −0.301 | 1.00 | 16.82 | A |
| ATOM | 3068 | CG | PRO | A | 398 | 25.741 | 39.246 | 0.916 | 1.00 | 16.85 | A |
| ATOM | 3069 | C | PRO | A | 398 | 27.726 | 38.392 | −1.282 | 1.00 | 15.76 | A |
| ATOM | 3070 | O | PRO | A | 398 | 28.589 | 39.044 | −0.684 | 1.00 | 15.25 | A |
| ATOM | 3071 | N | THR | A | 399 | 27.670 | 38.296 | −2.607 | 1.00 | 14.50 | A |
| ATOM | 3072 | CA | THR | A | 399 | 28.603 | 38.994 | −3.481 | 1.00 | 13.87 | A |
| ATOM | 3073 | CB | THR | A | 399 | 29.006 | 38.126 | −4.684 | 1.00 | 13.66 | A |
| ATOM | 3074 | OG1 | THR | A | 399 | 27.828 | 37.691 | −5.378 | 1.00 | 12.89 | A |
| ATOM | 3075 | CG2 | THR | A | 399 | 29.803 | 36.920 | −4.221 | 1.00 | 14.64 | A |
| ATOM | 3076 | C | THR | A | 399 | 27.881 | 40.238 | −3.986 | 1.00 | 13.35 | A |
| ATOM | 3077 | O | THR | A | 399 | 26.653 | 40.254 | −4.082 | 1.00 | 13.12 | A |
| ATOM | 3078 | N | LEU | A | 400 | 28.636 | 41.280 | −4.311 | 1.00 | 12.55 | A |
| ATOM | 3079 | CA | LEU | A | 400 | 28.019 | 42.517 | −4.776 | 1.00 | 11.68 | A |
| ATOM | 3080 | CB | LEU | A | 400 | 27.612 | 43.364 | −3.559 | 1.00 | 11.72 | A |
| ATOM | 3081 | CG | LEU | A | 400 | 26.954 | 44.740 | −3.743 | 1.00 | 11.84 | A |
| ATOM | 3082 | CD1 | LEU | A | 400 | 26.178 | 45.085 | −2.489 | 1.00 | 11.84 | A |
| ATOM | 3083 | CD2 | LEU | A | 400 | 28.002 | 45.812 | −4.032 | 1.00 | 11.75 | A |
| ATOM | 3084 | C | LEU | A | 400 | 28.941 | 43.317 | −5.678 | 1.00 | 11.82 | A |
| ATOM | 3085 | O | LEU | A | 400 | 30.160 | 43.294 | −5.508 | 1.00 | 10.86 | A |
| ATOM | 3086 | N | SER | A | 401 | 28.354 | 43.998 | −6.660 | 1.00 | 11.77 | A |
| ATOM | 3087 | CA | SER | A | 401 | 29.117 | 44.857 | −7.562 | 1.00 | 11.13 | A |
| ATOM | 3088 | CB | SER | A | 401 | 29.352 | 44.188 | −8.924 | 1.00 | 11.81 | A |
| ATOM | 3089 | OG | SER | A | 401 | 28.186 | 44.215 | −9.725 | 1.00 | 11.97 | A |
| ATOM | 3090 | C | SER | A | 401 | 28.283 | 46.123 | −7.739 | 1.00 | 11.29 | A |
| ATOM | 3091 | O | SER | A | 401 | 27.062 | 46.092 | −7.564 | 1.00 | 11.28 | A |
| ATOM | 3092 | N | GLY | A | 402 | 28.942 | 47.230 | −8.072 | 1.00 | 10.51 | A |
| ATOM | 3093 | CA | GLY | A | 402 | 28.245 | 48.492 | −8.256 | 1.00 | 11.12 | A |
| ATOM | 3094 | C | GLY | A | 402 | 28.827 | 49.576 | −7.362 | 1.00 | 11.67 | A |
| ATOM | 3095 | O | GLY | A | 402 | 29.852 | 49.361 | −6.715 | 1.00 | 12.01 | A |
| ATOM | 3096 | N | ASP | A | 403 | 28.183 | 50.739 | −7.322 | 1.00 | 11.30 | A |
| ATOM | 3097 | CA | ASP | A | 403 | 28.665 | 51.834 | −6.489 | 1.00 | 11.42 | A |
| ATOM | 3098 | CB | ASP | A | 403 | 29.180 | 52.983 | −7.367 | 1.00 | 11.48 | A |
| ATOM | 3099 | CG | ASP | A | 403 | 28.063 | 53.737 | −8.067 | 1.00 | 12.11 | A |
| ATOM | 3100 | OD1 | ASP | A | 403 | 26.967 | 53.168 | −8.236 | 1.00 | 13.59 | A |
| ATOM | 3101 | OD2 | ASP | A | 403 | 28.287 | 54.900 | −8.461 | 1.00 | 12.95 | A |
| ATOM | 3102 | C | ASP | A | 403 | 27.556 | 52.333 | −5.569 | 1.00 | 11.75 | A |
| ATOM | 3103 | O | ASP | A | 403 | 26.444 | 51.797 | −5.570 | 1.00 | 11.42 | A |
| ATOM | 3104 | N | PHE | A | 404 | 27.864 | 53.355 | −4.780 | 1.00 | 11.12 | A |
| ATOM | 3105 | CA | PHE | A | 404 | 26.887 | 53.913 | −3.865 | 1.00 | 11.24 | A |
| ATOM | 3106 | CB | PHE | A | 404 | 27.233 | 53.526 | −2.424 | 1.00 | 11.12 | A |
| ATOM | 3107 | CG | PHE | A | 404 | 27.204 | 52.036 | −2.184 | 1.00 | 11.08 | A |
| ATOM | 3108 | CD1 | PHE | A | 404 | 28.377 | 51.288 | −2.207 | 1.00 | 10.94 | A |
| ATOM | 3109 | CD2 | PHE | A | 404 | 25.993 | 51.378 | −1.980 | 1.00 | 10.57 | A |
| ATOM | 3110 | CE1 | PHE | A | 404 | 28.347 | 49.899 | −2.030 | 1.00 | 11.95 | A |
| ATOM | 3111 | CE2 | PHE | A | 404 | 25.949 | 49.992 | −1.802 | 1.00 | 10.67 | A |
| ATOM | 3112 | CZ | PHE | A | 404 | 27.126 | 49.250 | −1.826 | 1.00 | 11.36 | A |
| ATOM | 3113 | C | PHE | A | 404 | 26.737 | 55.422 | −4.003 | 1.00 | 11.30 | A |
| ATOM | 3114 | O | PHE | A | 404 | 26.912 | 56.178 | −3.046 | 1.00 | 10.08 | A |
| ATOM | 3115 | N | PHE | A | 405 | 26.420 | 55.835 | −5.228 | 1.00 | 11.82 | A |
| ATOM | 3116 | CA | PHE | A | 405 | 26.172 | 57.230 | −5.575 | 1.00 | 12.35 | A |
| ATOM | 3117 | CB | PHE | A | 405 | 27.235 | 57.750 | −6.550 | 1.00 | 12.94 | A |
| ATOM | 3118 | CG | PHE | A | 405 | 28.614 | 57.857 | −5.958 | 1.00 | 13.35 | A |
| ATOM | 3119 | CD1 | PHE | A | 405 | 29.699 | 57.250 | −6.585 | 1.00 | 13.75 | A |
| ATOM | 3120 | CD2 | PHE | A | 405 | 28.837 | 58.584 | −4.789 | 1.00 | 13.48 | A |
| ATOM | 3121 | CE1 | PHE | A | 405 | 30.987 | 57.366 | −6.057 | 1.00 | 12.89 | A |
| ATOM | 3122 | CE2 | PHE | A | 405 | 30.126 | 58.706 | −4.255 | 1.00 | 12.99 | A |
| ATOM | 3123 | CZ | PHE | A | 405 | 31.198 | 58.095 | −4.891 | 1.00 | 12.93 | A |
| ATOM | 3124 | C | PHE | A | 405 | 24.817 | 57.170 | −6.290 | 1.00 | 12.41 | A |
| ATOM | 3125 | O | PHE | A | 405 | 24.539 | 56.187 | −6.972 | 1.00 | 12.49 | A |
| ATOM | 3126 | N | THR | A | 406 | 23.973 | 58.192 | −6.153 | 1.00 | 12.18 | A |
| ATOM | 3127 | CA | THR | A | 406 | 24.254 | 59.382 | −5.364 | 1.00 | 12.39 | A |
| ATOM | 3128 | CB | THR | A | 406 | 23.747 | 60.646 | −6.108 | 1.00 | 12.21 | A |
| ATOM | 3129 | OG1 | THR | A | 406 | 24.631 | 60.929 | −7.201 | 1.00 | 13.40 | A |
| ATOM | 3130 | CG2 | THR | A | 406 | 23.683 | 61.848 | −5.177 | 1.00 | 12.11 | A |
| ATOM | 3131 | C | THR | A | 406 | 23.611 | 59.280 | −3.983 | 1.00 | 12.64 | A |
| ATOM | 3132 | O | THR | A | 406 | 22.440 | 58.915 | −3.838 | 1.00 | 12.69 | A |
| ATOM | 3133 | N | TYR | A | 407 | 24.404 | 59.604 | −2.971 | 1.00 | 12.81 | A |
| ATOM | 3134 | CA | TYR | A | 407 | 23.985 | 59.559 | −1.578 | 1.00 | 12.99 | A |
| ATOM | 3135 | CB | TYR | A | 407 | 25.207 | 59.809 | −0.697 | 1.00 | 12.23 | A |
| ATOM | 3136 | CG | TYR | A | 407 | 24.941 | 59.972 | 0.786 | 1.00 | 13.17 | A |
| ATOM | 3137 | CD1 | TYR | A | 407 | 24.456 | 58.914 | 1.555 | 1.00 | 12.48 | A |
| ATOM | 3138 | CE1 | TYR | A | 407 | 24.326 | 59.033 | 2.942 | 1.00 | 12.87 | A |
| ATOM | 3139 | CD2 | TYR | A | 407 | 25.277 | 61.160 | 1.436 | 1.00 | 12.63 | A |
| ATOM | 3140 | CE2 | TYR | A | 407 | 25.154 | 61.290 | 2.812 | 1.00 | 12.47 | A |
| ATOM | 3141 | CZ | TYR | A | 407 | 24.686 | 60.226 | 3.562 | 1.00 | 12.24 | A |
| ATOM | 3142 | OH | TYR | A | 407 | 24.637 | 60.347 | 4.933 | 1.00 | 11.95 | A |
| ATOM | 3143 | C | TYR | A | 407 | 22.890 | 60.554 | −1.211 | 1.00 | 13.79 | A |
| ATOM | 3144 | O | TYR | A | 407 | 22.830 | 61.664 | −1.742 | 1.00 | 13.92 | A |
| ATOM | 3145 | N | ALA | A | 408 | 22.026 | 60.129 | −0.294 | 1.00 | 14.16 | A |
| ATOM | 3146 | CA | ALA | A | 408 | 20.942 | 60.948 | 0.234 | 1.00 | 14.28 | A |
| ATOM | 3147 | CB | ALA | A | 408 | 19.632 | 60.667 | −0.505 | 1.00 | 14.27 | A |
| ATOM | 3148 | C | ALA | A | 408 | 20.839 | 60.506 | 1.685 | 1.00 | 14.52 | A |
| ATOM | 3149 | O | ALA | A | 408 | 20.688 | 59.313 | 1.956 | 1.00 | 14.73 | A |
| ATOM | 3150 | N | ASP | A | 409 | 20.955 | 61.444 | 2.622 | 1.00 | 14.31 | A |
| ATOM | 3151 | CA | ASP | A | 409 | 20.881 | 61.082 | 4.031 | 1.00 | 14.91 | A |
| ATOM | 3152 | CB | ASP | A | 409 | 21.835 | 61.956 | 4.870 | 1.00 | 14.46 | A |
| ATOM | 3153 | CG | ASP | A | 409 | 21.512 | 63.441 | 4.803 | 1.00 | 14.85 | A |
| ATOM | 3154 | OD1 | ASP | A | 409 | 20.939 | 63.893 | 3.791 | 1.00 | 13.80 | A |
| ATOM | 3155 | OD2 | ASP | A | 409 | 21.860 | 64.164 | 5.768 | 1.00 | 14.51 | A |
| ATOM | 3156 | C | ASP | A | 409 | 19.455 | 61.135 | 4.573 | 1.00 | 15.58 | A |
| ATOM | 3157 | O | ASP | A | 409 | 19.159 | 60.546 | 5.610 | 1.00 | 15.51 | A |
| ATOM | 3158 | N | ARG | A | 410 | 18.573 | 61.825 | 3.853 | 1.00 | 16.53 | A |
| ATOM | 3159 | CA | ARG | A | 410 | 17.167 | 61.927 | 4.240 | 1.00 | 17.84 | A |
| ATOM | 3160 | CB | ARG | A | 410 | 17.008 | 62.732 | 5.535 | 1.00 | 19.59 | A |
| ATOM | 3161 | CG | ARG | A | 410 | 17.450 | 64.188 | 5.475 | 1.00 | 22.30 | A |
| ATOM | 3162 | CD | ARG | A | 410 | 17.305 | 64.806 | 6.861 | 1.00 | 25.55 | A |
| ATOM | 3163 | NE | ARG | A | 410 | 17.958 | 66.105 | 7.004 | 1.00 | 28.63 | A |
| ATOM | 3164 | CZ | ARG | A | 410 | 17.454 | 67.258 | 6.577 | 1.00 | 30.10 | A |
| ATOM | 3165 | NH1 | ARG | A | 410 | 16.273 | 67.292 | 5.967 | 1.00 | 31.30 | A |
| ATOM | 3166 | NH2 | ARG | A | 410 | 18.131 | 68.383 | 6.772 | 1.00 | 29.89 | A |
| ATOM | 3167 | C | ARG | A | 410 | 16.320 | 62.559 | 3.139 | 1.00 | 17.93 | A |
| ATOM | 3168 | O | ARG | A | 410 | 16.824 | 63.314 | 2.305 | 1.00 | 17.10 | A |
| ATOM | 3169 | N | SER | A | 411 | 15.031 | 62.230 | 3.150 | 1.00 | 17.62 | A |
| ATOM | 3170 | CA | SER | A | 411 | 14.061 | 62.737 | 2.182 | 1.00 | 18.15 | A |
| ATOM | 3171 | CB | SER | A | 411 | 13.513 | 64.094 | 2.647 | 1.00 | 19.65 | A |
| ATOM | 3172 | OG | SER | A | 411 | 14.555 | 65.020 | 2.893 | 1.00 | 22.94 | A |
| ATOM | 3173 | C | SER | A | 411 | 14.586 | 62.846 | 0.754 | 1.00 | 16.96 | A |
| ATOM | 3174 | O | SER | A | 411 | 15.010 | 61.850 | 0.162 | 1.00 | 16.97 | A |
| ATOM | 3175 | N | ASP | A | 412 | 14.538 | 64.053 | 0.198 | 1.00 | 15.76 | A |
| ATOM | 3176 | CA | ASP | A | 412 | 15.002 | 64.289 | −1.165 | 1.00 | 15.28 | A |
| ATOM | 3177 | CB | ASP | A | 412 | 13.967 | 65.119 | −1.939 | 1.00 | 15.42 | A |
| ATOM | 3178 | CG | ASP | A | 412 | 13.836 | 66.545 | −1.408 | 1.00 | 16.05 | A |
| ATOM | 3179 | OD1 | ASP | A | 412 | 14.311 | 66.820 | −0.284 | 1.00 | 15.81 | A |
| ATOM | 3180 | OD2 | ASP | A | 412 | 13.243 | 67.390 | −2.117 | 1.00 | 15.35 | A |
| ATOM | 3181 | C | ASP | A | 412 | 16.346 | 65.014 | −1.174 | 1.00 | 14.37 | A |
| ATOM | 3182 | O | ASP | A | 412 | 16.756 | 65.550 | −2.200 | 1.00 | 14.24 | A |
| ATOM | 3183 | N | ASN | A | 413 | 17.022 | 65.026 | −0.029 | 1.00 | 14.32 | A |
| ATOM | 3184 | CA | ASN | A | 413 | 18.315 | 65.697 | 0.091 | 1.00 | 13.86 | A |
| ATOM | 3185 | CB | ASN | A | 413 | 18.632 | 66.010 | 1.557 | 1.00 | 14.09 | A |
| ATOM | 3186 | CG | ASN | A | 413 | 17.723 | 67.078 | 2.155 | 1.00 | 14.34 | A |
| ATOM | 3187 | OD1 | ASN | A | 413 | 17.958 | 67.540 | 3.270 | 1.00 | 15.45 | A |
| ATOM | 3188 | ND2 | ASN | A | 413 | 16.685 | 67.465 | 1.425 | 1.00 | 13.77 | A |
| ATOM | 3189 | C | ASN | A | 413 | 19.453 | 64.857 | −0.488 | 1.00 | 14.05 | A |
| ATOM | 3190 | O | ASN | A | 413 | 20.151 | 64.161 | 0.252 | 1.00 | 13.05 | A |
| ATOM | 3191 | N | TYR | A | 414 | 19.631 | 64.926 | −1.806 | 1.00 | 13.36 | A |
| ATOM | 3192 | CA | TYR | A | 414 | 20.696 | 64.186 | −2.478 | 1.00 | 13.34 | A |
| ATOM | 3193 | CB | TYR | A | 414 | 20.265 | 63.760 | −3.886 | 1.00 | 13.11 | A |
| ATOM | 3194 | CG | TYR | A | 414 | 19.261 | 62.627 | −3.894 | 1.00 | 12.95 | A |
| ATOM | 3195 | CD1 | TYR | A | 414 | 17.910 | 62.856 | −3.619 | 1.00 | 13.47 | A |
| ATOM | 3196 | CE1 | TYR | A | 414 | 16.990 | 61.802 | −3.601 | 1.00 | 13.24 | A |
| ATOM | 3197 | CD2 | TYR | A | 414 | 19.669 | 61.316 | −4.149 | 1.00 | 12.99 | A |
| ATOM | 3198 | CE2 | TYR | A | 414 | 18.761 | 60.260 | −4.129 | 1.00 | 12.68 | A |
| ATOM | 3199 | CZ | TYR | A | 414 | 17.425 | 60.508 | −3.857 | 1.00 | 13.38 | A |
| ATOM | 3200 | OH | TYR | A | 414 | 16.531 | 59.457 | −3.848 | 1.00 | 12.59 | A |
| ATOM | 3201 | C | TYR | A | 414 | 21.940 | 65.067 | −2.554 | 1.00 | 12.83 | A |
| ATOM | 3202 | O | TYR | A | 414 | 21.867 | 66.235 | −2.941 | 1.00 | 12.53 | A |
| ATOM | 3203 | N | TRP | A | 415 | 23.078 | 64.490 | −2.188 | 1.00 | 12.46 | A |
| ATOM | 3204 | CA | TRP | A | 415 | 24.343 | 65.213 | −2.152 | 1.00 | 12.74 | A |
| ATOM | 3205 | CB | TRP | A | 415 | 25.250 | 64.590 | −1.088 | 1.00 | 12.36 | A |
| ATOM | 3206 | CG | TRP | A | 415 | 24.676 | 64.612 | 0.297 | 1.00 | 12.97 | A |
| ATOM | 3207 | CD2 | TRP | A | 415 | 25.388 | 64.862 | 1.512 | 1.00 | 13.40 | A |
| ATOM | 3208 | CE2 | TRP | A | 415 | 24.461 | 64.742 | 2.573 | 1.00 | 13.64 | A |
| ATOM | 3209 | CE3 | TRP | A | 415 | 26.723 | 65.175 | 1.810 | 1.00 | 13.48 | A |
| ATOM | 3210 | CD1 | TRP | A | 415 | 23.379 | 64.356 | 0.658 | 1.00 | 12.64 | A |
| ATOM | 3211 | NE1 | TRP | A | 415 | 23.242 | 64.434 | 2.024 | 1.00 | 13.04 | A |
| ATOM | 3212 | CZ2 | TRP | A | 415 | 24.827 | 64.923 | 3.911 | 1.00 | 13.72 | A |
| ATOM | 3213 | CZ3 | TRP | A | 415 | 27.086 | 65.354 | 3.135 | 1.00 | 13.13 | A |
| ATOM | 3214 | CH2 | TRP | A | 415 | 26.139 | 65.227 | 4.174 | 1.00 | 13.76 | A |
| ATOM | 3215 | C | TRP | A | 415 | 25.086 | 65.255 | −3.482 | 1.00 | 12.39 | A |
| ATOM | 3216 | O | TRP | A | 415 | 26.224 | 64.805 | −3.566 | 1.00 | 12.69 | A |
| ATOM | 3217 | N | SER | A | 416 | 24.453 | 65.795 | −4.516 | 1.00 | 11.73 | A |
| ATOM | 3218 | CA | SER | A | 416 | 25.102 | 65.878 | −5.815 | 1.00 | 11.54 | A |
| ATOM | 3219 | CB | SER | A | 416 | 24.117 | 65.508 | −6.932 | 1.00 | 11.23 | A |
| ATOM | 3220 | OG | SER | A | 416 | 22.849 | 66.106 | −6.727 | 1.00 | 10.80 | A |
| ATOM | 3221 | C | SER | A | 416 | 25.678 | 67.271 | −6.046 | 1.00 | 11.26 | A |
| ATOM | 3222 | O | SER | A | 416 | 26.311 | 67.526 | −7.063 | 1.00 | 11.02 | A |
| ATOM | 3223 | N | GLY | A | 417 | 25.468 | 68.165 | −5.085 | 1.00 | 12.11 | A |
| ATOM | 3224 | CA | GLY | A | 417 | 25.983 | 69.518 | −5.214 | 1.00 | 12.23 | A |
| ATOM | 3225 | C | GLY | A | 417 | 27.500 | 69.582 | −5.147 | 1.00 | 12.12 | A |
| ATOM | 3226 | O | GLY | A | 417 | 28.130 | 70.296 | −5.930 | 1.00 | 12.13 | A |
| ATOM | 3227 | N | TYR | A | 418 | 28.090 | 68.818 | −4.230 | 1.00 | 11.66 | A |
| ATOM | 3228 | CA | TYR | A | 418 | 29.541 | 68.813 | −4.051 | 1.00 | 11.15 | A |
| ATOM | 3229 | CB | TYR | A | 418 | 29.904 | 68.106 | −2.738 | 1.00 | 10.71 | A |
| ATOM | 3230 | CG | TYR | A | 418 | 30.049 | 66.597 | −2.808 | 1.00 | 11.18 | A |
| ATOM | 3231 | CD1 | TYR | A | 418 | 31.302 | 66.008 | −2.993 | 1.00 | 10.96 | A |
| ATOM | 3232 | CE1 | TYR | A | 418 | 31.456 | 64.616 | −2.990 | 1.00 | 10.46 | A |
| ATOM | 3233 | CD2 | TYR | A | 418 | 28.947 | 65.758 | −2.632 | 1.00 | 11.76 | A |
| ATOM | 3234 | CE2 | TYR | A | 418 | 29.090 | 64.360 | −2.628 | 1.00 | 11.02 | A |
| ATOM | 3235 | CZ | TYR | A | 418 | 30.348 | 63.803 | −2.805 | 1.00 | 10.28 | A |
| ATOM | 3236 | OH | TYR | A | 418 | 30.511 | 62.435 | −2.778 | 1.00 | 11.20 | A |
| ATOM | 3237 | C | TYR | A | 418 | 30.313 | 68.207 | −5.231 | 1.00 | 10.98 | A |
| ATOM | 3238 | O | TYR | A | 418 | 31.545 | 68.208 | −5.250 | 1.00 | 10.37 | A |
| ATOM | 3239 | N | TYR | A | 419 | 29.591 | 67.683 | −6.213 | 1.00 | 10.63 | A |
| ATOM | 3240 | CA | TYR | A | 419 | 30.245 | 67.144 | −7.399 | 1.00 | 10.39 | A |
| ATOM | 3241 | CB | TYR | A | 419 | 29.247 | 66.380 | −8.280 | 1.00 | 10.83 | A |
| ATOM | 3242 | CG | TYR | A | 419 | 28.624 | 65.146 | −7.656 | 1.00 | 10.85 | A |
| ATOM | 3243 | CD1 | TYR | A | 419 | 27.464 | 64.590 | −8.198 | 1.00 | 11.08 | A |
| ATOM | 3244 | CE1 | TYR | A | 419 | 26.897 | 63.438 | −7.660 | 1.00 | 11.16 | A |
| ATOM | 3245 | CD2 | TYR | A | 419 | 29.201 | 64.518 | −6.555 | 1.00 | 10.49 | A |
| ATOM | 3246 | CE2 | TYR | A | 419 | 28.640 | 63.359 | −6.008 | 1.00 | 10.86 | A |
| ATOM | 3247 | CZ | TYR | A | 419 | 27.489 | 62.827 | −6.568 | 1.00 | 11.11 | A |
| ATOM | 3248 | OH | TYR | A | 419 | 26.935 | 61.675 | −6.051 | 1.00 | 10.77 | A |
| ATOM | 3249 | C | TYR | A | 419 | 30.766 | 68.351 | −8.191 | 1.00 | 10.55 | A |
| ATOM | 3250 | O | TYR | A | 419 | 31.607 | 68.203 | −9.083 | 1.00 | 11.05 | A |
| ATOM | 3251 | N | THR | A | 420 | 30.279 | 69.544 | −7.843 | 1.00 | 10.27 | A |
| ATOM | 3252 | CA | THR | A | 420 | 30.663 | 70.770 | −8.548 | 1.00 | 9.65 | A |
| ATOM | 3253 | CB | THR | A | 420 | 29.458 | 71.312 | −9.355 | 1.00 | 10.18 | A |
| ATOM | 3254 | OG1 | THR | A | 420 | 28.971 | 70.285 | −10.228 | 1.00 | 10.10 | A |
| ATOM | 3255 | CG2 | THR | A | 420 | 29.859 | 72.532 | −10.190 | 1.00 | 9.96 | A |
| ATOM | 3256 | C | THR | A | 420 | 31.224 | 71.918 | −7.699 | 1.00 | 10.48 | A |
| ATOM | 3257 | O | THR | A | 420 | 32.033 | 72.708 | −8.187 | 1.00 | 10.35 | A |
| ATOM | 3258 | N | SER | A | 421 | 30.798 | 72.009 | −6.440 | 1.00 | 10.65 | A |
| ATOM | 3259 | CA | SER | A | 421 | 31.240 | 73.077 | −5.540 | 1.00 | 10.83 | A |
| ATOM | 3260 | CB | SER | A | 421 | 30.851 | 72.737 | −4.099 | 1.00 | 9.91 | A |
| ATOM | 3261 | OG | SER | A | 421 | 29.446 | 72.581 | −3.991 | 1.00 | 11.04 | A |
| ATOM | 3262 | C | SER | A | 421 | 32.733 | 73.390 | −5.607 | 1.00 | 10.88 | A |
| ATOM | 3263 | O | SER | A | 421 | 33.571 | 72.483 | −5.612 | 1.00 | 10.61 | A |
| ATOM | 3264 | N | ARG | A | 422 | 33.051 | 74.685 | −5.637 | 1.00 | 11.06 | A |
| ATOM | 3265 | CA | ARG | A | 422 | 34.434 | 75.158 | −5.717 | 1.00 | 11.32 | A |
| ATOM | 3266 | CB | ARG | A | 422 | 35.163 | 74.880 | −4.400 | 1.00 | 12.09 | A |
| ATOM | 3267 | CG | ARG | A | 422 | 34.966 | 75.954 | −3.316 | 1.00 | 12.90 | A |
| ATOM | 3268 | CD | ARG | A | 422 | 33.507 | 76.183 | −2.913 | 1.00 | 13.25 | A |
| ATOM | 3269 | NE | ARG | A | 422 | 33.436 | 77.150 | −1.813 | 1.00 | 14.01 | A |
| ATOM | 3270 | CZ | ARG | A | 422 | 33.503 | 76.829 | −0.524 | 1.00 | 14.36 | A |
| ATOM | 3271 | NH1 | ARG | A | 422 | 33.619 | 75.563 | −0.153 | 1.00 | 14.25 | A |
| ATOM | 3272 | NH2 | ARG | A | 422 | 33.519 | 77.783 | 0.400 | 1.00 | 15.13 | A |
| ATOM | 3273 | C | ARG | A | 422 | 35.162 | 74.486 | −6.885 | 1.00 | 11.33 | A |
| ATOM | 3274 | O | ARG | A | 422 | 36.178 | 73.804 | −6.704 | 1.00 | 11.04 | A |
| ATOM | 3275 | N | PRO | A | 423 | 34.660 | 74.700 | −8.112 | 1.00 | 11.11 | A |
| ATOM | 3276 | CD | PRO | A | 423 | 33.538 | 75.594 | −8.455 | 1.00 | 10.63 | A |
| ATOM | 3277 | CA | PRO | A | 423 | 35.249 | 74.111 | −9.319 | 1.00 | 10.85 | A |
| ATOM | 3278 | CB | PRO | A | 423 | 34.251 | 74.496 | −10.410 | 1.00 | 11.30 | A |
| ATOM | 3279 | CG | PRO | A | 423 | 33.748 | 75.829 | −9.944 | 1.00 | 12.06 | A |
| ATOM | 3280 | C | PRO | A | 423 | 36.678 | 74.541 | −9.644 | 1.00 | 10.60 | A |
| ATOM | 3281 | O | PRO | A | 423 | 37.405 | 73.817 | −10.320 | 1.00 | 9.70 | A |
| ATOM | 3282 | N | TYR | A | 424 | 37.082 | 75.717 | −9.175 | 1.00 | 10.48 | A |
| ATOM | 3283 | CA | TYR | A | 424 | 38.439 | 76.192 | −9.433 | 1.00 | 11.22 | A |
| ATOM | 3284 | CB | TYR | A | 424 | 38.658 | 77.554 | −8.769 | 1.00 | 12.13 | A |
| ATOM | 3285 | CG | TYR | A | 424 | 40.028 | 78.140 | −9.029 | 1.00 | 12.96 | A |
| ATOM | 3286 | CD1 | TYR | A | 424 | 40.274 | 78.899 | −10.170 | 1.00 | 13.38 | A |
| ATOM | 3287 | CE1 | TYR | A | 424 | 41.533 | 79.433 | −10.421 | 1.00 | 14.39 | A |
| ATOM | 3288 | CD2 | TYR | A | 424 | 41.084 | 77.922 | −8.138 | 1.00 | 13.80 | A |
| ATOM | 3289 | CE2 | TYR | A | 424 | 42.355 | 78.450 | −8.383 | 1.00 | 14.53 | A |
| ATOM | 3290 | CZ | TYR | A | 424 | 42.568 | 79.206 | −9.526 | 1.00 | 14.85 | A |
| ATOM | 3291 | OH | TYR | A | 424 | 43.812 | 79.745 | −9.785 | 1.00 | 15.46 | A |
| ATOM | 3292 | C | TYR | A | 424 | 39.451 | 75.196 | −8.863 | 1.00 | 11.15 | A |
| ATOM | 3293 | O | TYR | A | 424 | 40.402 | 74.787 | −9.536 | 1.00 | 10.39 | A |
| ATOM | 3294 | N | HIS | A | 425 | 39.226 | 74.794 | −7.617 | 1.00 | 10.48 | A |
| ATOM | 3295 | CA | HIS | A | 425 | 40.125 | 73.872 | −6.933 | 1.00 | 10.34 | A |
| ATOM | 3296 | CB | HIS | A | 425 | 39.876 | 73.985 | −5.431 | 1.00 | 10.89 | A |
| ATOM | 3297 | CG | HIS | A | 425 | 39.851 | 75.404 | −4.959 | 1.00 | 11.17 | A |
| ATOM | 3298 | CD2 | HIS | A | 425 | 38.823 | 76.274 | −4.820 | 1.00 | 10.14 | A |
| ATOM | 3299 | ND1 | HIS | A | 425 | 41.000 | 76.127 | −4.719 | 1.00 | 11.79 | A |
| ATOM | 3300 | CE1 | HIS | A | 425 | 40.682 | 77.382 | −4.460 | 1.00 | 10.26 | A |
| ATOM | 3301 | NE2 | HIS | A | 425 | 39.368 | 77.499 | −4.516 | 1.00 | 12.42 | A |
| ATOM | 3302 | C | HIS | A | 425 | 39.985 | 72.437 | −7.431 | 1.00 | 10.16 | A |
| ATOM | 3303 | O | HIS | A | 425 | 40.941 | 71.660 | −7.372 | 1.00 | 9.68 | A |
| ATOM | 3304 | N | LYS | A | 426 | 38.800 | 72.088 | −7.923 | 1.00 | 10.04 | A |
| ATOM | 3305 | CA | LYS | A | 426 | 38.581 | 70.752 | −8.478 | 1.00 | 10.39 | A |
| ATOM | 3306 | CB | LYS | A | 426 | 37.106 | 70.563 | −8.869 | 1.00 | 10.40 | A |
| ATOM | 3307 | CG | LYS | A | 426 | 36.193 | 70.161 | −7.714 | 1.00 | 10.43 | A |
| ATOM | 3308 | CD | LYS | A | 426 | 34.724 | 70.107 | −8.150 | 1.00 | 10.31 | A |
| ATOM | 3309 | CE | LYS | A | 426 | 33.871 | 69.281 | −7.183 | 1.00 | 10.26 | A |
| ATOM | 3310 | NZ | LYS | A | 426 | 33.888 | 69.790 | −5.777 | 1.00 | 10.85 | A |
| ATOM | 3311 | C | LYS | A | 426 | 39.470 | 70.629 | −9.718 | 1.00 | 10.40 | A |
| ATOM | 3312 | O | LYS | A | 426 | 40.073 | 69.579 | −9.969 | 1.00 | 10.72 | A |
| ATOM | 3313 | N | ARG | A | 427 | 39.549 | 71.711 | −10.491 | 1.00 | 9.88 | A |
| ATOM | 3314 | CA | ARG | A | 427 | 40.379 | 71.736 | −11.691 | 1.00 | 10.35 | A |
| ATOM | 3315 | CB | ARG | A | 427 | 40.019 | 72.960 | −12.549 | 1.00 | 11.69 | A |
| ATOM | 3316 | CG | ARG | A | 427 | 40.997 | 73.290 | −13.671 | 1.00 | 12.53 | A |
| ATOM | 3317 | CD | ARG | A | 427 | 41.271 | 72.122 | −14.618 | 1.00 | 12.72 | A |
| ATOM | 3318 | NE | ARG | A | 427 | 42.265 | 72.520 | −15.613 | 1.00 | 12.83 | A |
| ATOM | 3319 | CZ | ARG | A | 427 | 43.039 | 71.683 | −16.293 | 1.00 | 12.73 | A |
| ATOM | 3320 | NH1 | ARG | A | 427 | 42.948 | 70.371 | −16.103 | 1.00 | 12.04 | A |
| ATOM | 3321 | NH2 | ARG | A | 427 | 43.928 | 72.167 | −17.152 | 1.00 | 13.32 | A |
| ATOM | 3322 | C | ARG | A | 427 | 41.849 | 71.774 | −11.268 | 1.00 | 10.25 | A |
| ATOM | 3323 | O | ARG | A | 427 | 42.695 | 71.101 | −11.852 | 1.00 | 9.51 | A |
| ATOM | 3324 | N | MET | A | 428 | 42.145 | 72.546 | −10.230 | 1.00 | 10.68 | A |
| ATOM | 3325 | CA | MET | A | 428 | 43.514 | 72.644 | −9.738 | 1.00 | 11.11 | A |
| ATOM | 3326 | CB | MET | A | 428 | 43.571 | 73.614 | −8.556 | 1.00 | 12.20 | A |
| ATOM | 3327 | CG | MET | A | 428 | 44.976 | 73.931 | −8.088 | 1.00 | 13.51 | A |
| ATOM | 3328 | SD | MET | A | 428 | 45.000 | 75.304 | −6.918 | 1.00 | 15.32 | A |
| ATOM | 3329 | CE | MET | A | 428 | 46.751 | 75.650 | −6.870 | 1.00 | 14.47 | A |
| ATOM | 3330 | C | MET | A | 428 | 44.019 | 71.258 | −9.321 | 1.00 | 10.53 | A |
| ATOM | 3331 | O | MET | A | 428 | 45.199 | 70.934 | −9.502 | 1.00 | 9.33 | A |
| ATOM | 3332 | N | ASP | A | 429 | 43.118 | 70.438 | −8.780 | 1.00 | 9.95 | A |
| ATOM | 3333 | CA | ASP | A | 429 | 43.475 | 69.086 | −8.352 | 1.00 | 9.37 | A |
| ATOM | 3334 | CB | ASP | A | 429 | 42.251 | 68.358 | −7.788 | 1.00 | 10.10 | A |
| ATOM | 3335 | CG | ASP | A | 429 | 42.535 | 66.894 | −7.467 | 1.00 | 10.35 | A |
| ATOM | 3336 | OD1 | ASP | A | 429 | 42.230 | 66.024 | −8.318 | 1.00 | 10.71 | A |
| ATOM | 3337 | OD2 | ASP | A | 429 | 43.069 | 66.616 | −6.372 | 1.00 | 9.82 | A |
| ATOM | 3338 | C | ASP | A | 429 | 44.063 | 68.268 | −9.496 | 1.00 | 9.63 | A |
| ATOM | 3339 | O | ASP | A | 429 | 45.084 | 67.598 | −9.332 | 1.00 | 8.73 | A |
| ATOM | 3340 | N | ARG | A | 430 | 43.417 | 68.328 | −10.658 | 1.00 | 9.03 | A |
| ATOM | 3341 | CA | ARG | A | 430 | 43.876 | 67.573 | −11.820 | 1.00 | 8.89 | A |
| ATOM | 3342 | CB | ARG | A | 430 | 42.805 | 67.604 | −12.911 | 1.00 | 9.00 | A |
| ATOM | 3343 | CG | ARG | A | 430 | 41.515 | 66.914 | −12.507 | 1.00 | 9.05 | A |
| ATOM | 3344 | CD | ARG | A | 430 | 41.750 | 65.448 | −12.150 | 1.00 | 9.51 | A |
| ATOM | 3345 | NE | ARG | A | 430 | 40.505 | 64.684 | −12.128 | 1.00 | 9.34 | A |
| ATOM | 3346 | CZ | ARG | A | 430 | 39.818 | 64.382 | −11.029 | 1.00 | 10.40 | A |
| ATOM | 3347 | NH1 | ARG | A | 430 | 40.248 | 64.770 | −9.837 | 1.00 | 9.53 | A |
| ATOM | 3348 | NH2 | ARG | A | 430 | 38.683 | 63.695 | −11.129 | 1.00 | 10.19 | A |
| ATOM | 3349 | C | ARG | A | 430 | 45.201 | 68.087 | −12.371 | 1.00 | 9.55 | A |
| ATOM | 3350 | O | ARG | A | 430 | 46.020 | 67.312 | −12.878 | 1.00 | 8.82 | A |
| ATOM | 3351 | N | VAL | A | 431 | 45.407 | 69.396 | −12.282 | 1.00 | 8.68 | A |
| ATOM | 3352 | CA | VAL | A | 431 | 46.652 | 69.989 | −12.751 | 1.00 | 10.08 | A |
| ATOM | 3353 | CB | VAL | A | 431 | 46.580 | 71.537 | −12.726 | 1.00 | 10.23 | A |
| ATOM | 3354 | CG1 | VAL | A | 431 | 47.954 | 72.137 | −13.037 | 1.00 | 11.41 | A |
| ATOM | 3355 | CG2 | VAL | A | 431 | 45.556 | 72.025 | −13.745 | 1.00 | 9.77 | A |
| ATOM | 3356 | C | VAL | A | 431 | 47.790 | 69.516 | −11.840 | 1.00 | 9.68 | A |
| ATOM | 3357 | O | VAL | A | 431 | 48.822 | 69.046 | −12.313 | 1.00 | 9.76 | A |
| ATOM | 3358 | N | LEU | A | 432 | 47.595 | 69.628 | −10.531 | 1.00 | 9.64 | A |
| ATOM | 3359 | CA | LEU | A | 432 | 48.632 | 69.206 | −9.596 | 1.00 | 9.75 | A |
| ATOM | 3360 | CB | LEU | A | 432 | 48.255 | 69.588 | −8.161 | 1.00 | 9.87 | A |
| ATOM | 3361 | CG | LEU | A | 432 | 49.292 | 69.268 | −7.079 | 1.00 | 9.92 | A |
| ATOM | 3362 | CD1 | LEU | A | 432 | 50.657 | 69.850 | −7.467 | 1.00 | 9.96 | A |
| ATOM | 3363 | CD2 | LEU | A | 432 | 48.825 | 69.849 | −5.738 | 1.00 | 9.99 | A |
| ATOM | 3364 | C | LEU | A | 432 | 48.865 | 67.703 | −9.698 | 1.00 | 9.62 | A |
| ATOM | 3365 | O | LEU | A | 432 | 49.998 | 67.237 | −9.587 | 1.00 | 10.09 | A |
| ATOM | 3366 | N | MET | A | 433 | 47.795 | 66.945 | −9.917 | 1.00 | 9.73 | A |
| ATOM | 3367 | CA | MET | A | 433 | 47.922 | 65.496 | −10.058 | 1.00 | 9.45 | A |
| ATOM | 3368 | CB | MET | A | 433 | 46.595 | 64.870 | −10.494 | 1.00 | 8.69 | A |
| ATOM | 3369 | CG | MET | A | 433 | 46.732 | 63.414 | −10.924 | 1.00 | 10.44 | A |
| ATOM | 3370 | SD | MET | A | 433 | 45.195 | 62.708 | −11.557 | 1.00 | 11.59 | A |
| ATOM | 3371 | CE | MET | A | 433 | 45.222 | 63.316 | −13.250 | 1.00 | 10.74 | A |
| ATOM | 3372 | C | MET | A | 433 | 48.972 | 65.188 | −11.117 | 1.00 | 9.60 | A |
| ATOM | 3373 | O | MET | A | 433 | 49.849 | 64.340 | −10.918 | 1.00 | 9.52 | A |
| ATOM | 3374 | N | HIS | A | 434 | 48.876 | 65.885 | −12.246 | 1.00 | 9.61 | A |
| ATOM | 3375 | CA | HIS | A | 434 | 49.813 | 65.676 | −13.342 | 1.00 | 9.91 | A |
| ATOM | 3376 | CB | HIS | A | 434 | 49.298 | 66.325 | −14.629 | 1.00 | 9.95 | A |
| ATOM | 3377 | CG | HIS | A | 434 | 50.281 | 66.257 | −15.752 | 1.00 | 10.58 | A |
| ATOM | 3378 | CD2 | HIS | A | 434 | 50.721 | 65.207 | −16.487 | 1.00 | 9.53 | A |
| ATOM | 3379 | ND1 | HIS | A | 434 | 51.018 | 67.347 | −16.161 | 1.00 | 11.20 | A |
| ATOM | 3380 | CE1 | HIS | A | 434 | 51.871 | 66.969 | −17.098 | 1.00 | 9.32 | A |
| ATOM | 3381 | NE2 | HIS | A | 434 | 51.712 | 65.677 | −17.313 | 1.00 | 11.60 | A |
| ATOM | 3382 | C | HIS | A | 434 | 51.214 | 66.191 | −13.048 | 1.00 | 10.24 | A |
| ATOM | 3383 | O | HIS | A | 434 | 52.202 | 65.538 | −13.391 | 1.00 | 9.05 | A |
| ATOM | 3384 | N | TYR | A | 435 | 51.301 | 67.370 | −12.434 | 1.00 | 10.57 | A |
| ATOM | 3385 | CA | TYR | A | 435 | 52.599 | 67.943 | −12.101 | 1.00 | 11.66 | A |
| ATOM | 3386 | CB | TYR | A | 435 | 52.436 | 69.314 | −11.436 | 1.00 | 13.23 | A |
| ATOM | 3387 | CG | TYR | A | 435 | 52.318 | 70.480 | −12.396 | 1.00 | 16.34 | A |
| ATOM | 3388 | CD1 | TYR | A | 435 | 51.340 | 70.506 | −13.391 | 1.00 | 17.44 | A |
| ATOM | 3389 | CE1 | TYR | A | 435 | 51.199 | 71.613 | −14.237 | 1.00 | 18.75 | A |
| ATOM | 3390 | CD2 | TYR | A | 435 | 53.157 | 71.588 | −12.270 | 1.00 | 18.76 | A |
| ATOM | 3391 | CE2 | TYR | A | 435 | 53.027 | 72.695 | −13.105 | 1.00 | 20.26 | A |
| ATOM | 3392 | CZ | TYR | A | 435 | 52.045 | 72.703 | −14.083 | 1.00 | 20.16 | A |
| ATOM | 3393 | OH | TYR | A | 435 | 51.903 | 73.818 | −14.884 | 1.00 | 22.28 | A |
| ATOM | 3394 | C | TYR | A | 435 | 53.364 | 67.018 | −11.161 | 1.00 | 11.02 | A |
| ATOM | 3395 | O | TYR | A | 435 | 54.578 | 66.868 | −11.290 | 1.00 | 10.53 | A |
| ATOM | 3396 | N | VAL | A | 436 | 52.658 | 66.400 | −10.215 | 1.00 | 9.43 | A |
| ATOM | 3397 | CA | VAL | A | 436 | 53.317 | 65.490 | −9.280 | 1.00 | 9.55 | A |
| ATOM | 3398 | CB | VAL | A | 436 | 52.341 | 65.008 | −8.175 | 1.00 | 8.98 | A |
| ATOM | 3399 | CG1 | VAL | A | 436 | 52.921 | 63.811 | −7.440 | 1.00 | 10.24 | A |
| ATOM | 3400 | CG2 | VAL | A | 436 | 52.094 | 66.145 | −7.185 | 1.00 | 8.57 | A |
| ATOM | 3401 | C | VAL | A | 436 | 53.885 | 64.295 | −10.039 | 1.00 | 10.04 | A |
| ATOM | 3402 | O | VAL | A | 436 | 55.043 | 63.917 | −9.849 | 1.00 | 9.58 | A |
| ATOM | 3403 | N | ARG | A | 437 | 53.076 | 63.707 | −10.914 | 1.00 | 9.63 | A |
| ATOM | 3404 | CA | ARG | A | 437 | 53.537 | 62.566 | −11.696 | 1.00 | 10.51 | A |
| ATOM | 3405 | CB | ARG | A | 437 | 52.416 | 62.050 | −12.607 | 1.00 | 10.60 | A |
| ATOM | 3406 | CG | ARG | A | 437 | 52.915 | 61.118 | −13.705 | 1.00 | 11.44 | A |
| ATOM | 3407 | CD | ARG | A | 437 | 51.778 | 60.538 | −14.544 | 1.00 | 11.25 | A |
| ATOM | 3408 | NE | ARG | A | 437 | 52.288 | 59.867 | −15.739 | 1.00 | 10.78 | A |
| ATOM | 3409 | CZ | ARG | A | 437 | 51.558 | 59.068 | −16.516 | 1.00 | 10.87 | A |
| ATOM | 3410 | NH1 | ARG | A | 437 | 50.285 | 58.831 | −16.224 | 1.00 | 10.01 | A |
| ATOM | 3411 | NH2 | ARG | A | 437 | 52.096 | 58.519 | −17.597 | 1.00 | 10.75 | A |
| ATOM | 3412 | C | ARG | A | 437 | 54.751 | 62.935 | −12.547 | 1.00 | 10.47 | A |
| ATOM | 3413 | O | ARG | A | 437 | 55.736 | 62.194 | −12.597 | 1.00 | 11.91 | A |
| ATOM | 3414 | N | ALA | A | 438 | 54.680 | 64.080 | −13.220 | 1.00 | 10.55 | A |
| ATOM | 3415 | CA | ALA | A | 438 | 55.771 | 64.523 | −14.086 | 1.00 | 10.56 | A |
| ATOM | 3416 | CB | ALA | A | 438 | 55.345 | 65.764 | −14.879 | 1.00 | 10.46 | A |
| ATOM | 3417 | C | ALA | A | 438 | 57.053 | 64.807 | −13.309 | 1.00 | 10.58 | A |
| ATOM | 3418 | O | ALA | A | 438 | 58.150 | 64.467 | −13.763 | 1.00 | 9.73 | A |
| ATOM | 3419 | N | ALA | A | 439 | 56.914 | 65.418 | −12.136 | 1.00 | 9.89 | A |
| ATOM | 3420 | CA | ALA | A | 439 | 58.074 | 65.738 | −11.310 | 1.00 | 9.47 | A |
| ATOM | 3421 | CB | ALA | A | 439 | 57.657 | 66.644 | −10.141 | 1.00 | 8.79 | A |
| ATOM | 3422 | C | ALA | A | 439 | 58.734 | 64.463 | −10.785 | 1.00 | 9.84 | A |
| ATOM | 3423 | O | ALA | A | 439 | 59.956 | 64.320 | −10.838 | 1.00 | 8.81 | A |
| ATOM | 3424 | N | GLU | A | 440 | 57.927 | 63.540 | −10.270 | 1.00 | 9.60 | A |
| ATOM | 3425 | CA | GLU | A | 440 | 58.469 | 62.283 | −9.757 | 1.00 | 10.28 | A |
| ATOM | 3426 | CB | GLU | A | 440 | 57.367 | 61.451 | −9.089 | 1.00 | 10.51 | A |
| ATOM | 3427 | CG | GLU | A | 440 | 56.796 | 62.085 | −7.833 | 1.00 | 11.25 | A |
| ATOM | 3428 | CD | GLU | A | 440 | 56.134 | 61.071 | −6.918 | 1.00 | 11.92 | A |
| ATOM | 3429 | OE1 | GLU | A | 440 | 55.012 | 60.614 | −7.222 | 1.00 | 12.12 | A |
| ATOM | 3430 | OE2 | GLU | A | 440 | 56.753 | 60.718 | −5.896 | 1.00 | 13.62 | A |
| ATOM | 3431 | C | GLU | A | 440 | 59.118 | 61.461 | −10.872 | 1.00 | 10.35 | A |
| ATOM | 3432 | O | GLU | A | 440 | 60.171 | 60.851 | −10.674 | 1.00 | 11.64 | A |
| ATOM | 3433 | N | MET | A | 441 | 58.494 | 61.437 | −12.045 | 1.00 | 10.26 | A |
| ATOM | 3434 | CA | MET | A | 441 | 59.047 | 60.666 | −13.158 | 1.00 | 10.07 | A |
| ATOM | 3435 | CB | MET | A | 441 | 58.010 | 60.511 | −14.274 | 1.00 | 9.51 | A |
| ATOM | 3436 | CG | MET | A | 441 | 58.520 | 59.763 | −15.507 | 1.00 | 9.93 | A |
| ATOM | 3437 | SD | MET | A | 441 | 57.250 | 59.554 | −16.783 | 1.00 | 10.71 | A |
| ATOM | 3438 | CE | MET | A | 441 | 56.191 | 58.321 | −16.011 | 1.00 | 11.36 | A |
| ATOM | 3439 | C | MET | A | 441 | 60.325 | 61.281 | −13.721 | 1.00 | 10.23 | A |
| ATOM | 3440 | O | MET | A | 441 | 61.322 | 60.584 | −13.913 | 1.00 | 9.58 | A |
| ATOM | 3441 | N | LEU | A | 442 | 60.304 | 62.585 | −13.982 | 1.00 | 10.26 | A |
| ATOM | 3442 | CA | LEU | A | 442 | 61.483 | 63.251 | −14.529 | 1.00 | 11.10 | A |
| ATOM | 3443 | CB | LEU | A | 442 | 61.175 | 64.719 | −14.846 | 1.00 | 11.61 | A |
| ATOM | 3444 | CG | LEU | A | 442 | 60.559 | 64.982 | −16.226 | 1.00 | 12.48 | A |
| ATOM | 3445 | CD1 | LEU | A | 442 | 59.975 | 66.389 | −16.271 | 1.00 | 12.40 | A |
| ATOM | 3446 | CD2 | LEU | A | 442 | 61.614 | 64.804 | −17.305 | 1.00 | 12.40 | A |
| ATOM | 3447 | C | LEU | A | 442 | 62.697 | 63.168 | −13.616 | 1.00 | 11.57 | A |
| ATOM | 3448 | O | LEU | A | 442 | 63.826 | 63.034 | −14.089 | 1.00 | 11.69 | A |
| ATOM | 3449 | N | SER | A | 443 | 62.474 | 63.228 | −12.307 | 1.00 | 11.46 | A |
| ATOM | 3450 | CA | SER | A | 443 | 63.590 | 63.172 | −11.373 | 1.00 | 11.37 | A |
| ATOM | 3451 | CB | SER | A | 443 | 63.257 | 63.967 | −10.104 | 1.00 | 11.11 | A |
| ATOM | 3452 | OG | SER | A | 443 | 62.166 | 63.396 | −9.405 | 1.00 | 10.78 | A |
| ATOM | 3453 | C | SER | A | 443 | 64.003 | 61.749 | −10.999 | 1.00 | 11.94 | A |
| ATOM | 3454 | O | SER | A | 443 | 65.064 | 61.545 | −10.403 | 1.00 | 11.73 | A |
| ATOM | 3455 | N | ALA | A | 444 | 63.177 | 60.769 | −11.360 | 1.00 | 11.28 | A |
| ATOM | 3456 | CA | ALA | A | 444 | 63.458 | 59.368 | −11.043 | 1.00 | 11.76 | A |
| ATOM | 3457 | CB | ALA | A | 444 | 62.204 | 58.521 | −11.264 | 1.00 | 11.73 | A |
| ATOM | 3458 | C | ALA | A | 444 | 64.618 | 58.782 | −11.846 | 1.00 | 12.36 | A |
| ATOM | 3459 | O | ALA | A | 444 | 65.267 | 57.835 | −11.402 | 1.00 | 12.49 | A |
| ATOM | 3460 | N | TRP | A | 445 | 64.880 | 59.345 | −13.021 | 1.00 | 12.37 | A |
| ATOM | 3461 | CA | TRP | A | 445 | 65.948 | 58.845 | −13.881 | 1.00 | 13.43 | A |
| ATOM | 3462 | CB | TRP | A | 445 | 65.945 | 59.593 | −15.218 | 1.00 | 12.38 | A |
| ATOM | 3463 | CG | TRP | A | 445 | 64.666 | 59.427 | −15.968 | 1.00 | 12.06 | A |
| ATOM | 3464 | CD2 | TRP | A | 445 | 64.217 | 58.246 | −16.645 | 1.00 | 11.74 | A |
| ATOM | 3465 | CE2 | TRP | A | 445 | 62.938 | 58.528 | −17.171 | 1.00 | 11.42 | A |
| ATOM | 3466 | CE3 | TRP | A | 445 | 64.772 | 56.975 | −16.857 | 1.00 | 12.15 | A |
| ATOM | 3467 | CD1 | TRP | A | 445 | 63.671 | 60.351 | −16.109 | 1.00 | 11.63 | A |
| ATOM | 3468 | NE1 | TRP | A | 445 | 62.630 | 59.819 | −16.829 | 1.00 | 11.30 | A |
| ATOM | 3469 | CZ2 | TRP | A | 445 | 62.198 | 57.584 | −17.901 | 1.00 | 11.44 | A |
| ATOM | 3470 | CZ3 | TRP | A | 445 | 64.037 | 56.034 | −17.581 | 1.00 | 12.95 | A |
| ATOM | 3471 | CH2 | TRP | A | 445 | 62.762 | 56.348 | −18.094 | 1.00 | 12.11 | A |
| ATOM | 3472 | C | TRP | A | 445 | 67.333 | 58.917 | −13.252 | 1.00 | 14.15 | A |
| ATOM | 3473 | O | TRP | A | 445 | 68.201 | 58.097 | −13.561 | 1.00 | 14.34 | A |
| ATOM | 3474 | N | HIS | A | 446 | 67.543 | 59.894 | −12.375 | 1.00 | 14.87 | A |
| ATOM | 3475 | CA | HIS | A | 446 | 68.831 | 60.046 | −11.709 | 1.00 | 15.74 | A |
| ATOM | 3476 | CB | HIS | A | 446 | 69.462 | 61.414 | −11.992 | 1.00 | 16.67 | A |
| ATOM | 3477 | CG | HIS | A | 446 | 69.875 | 61.625 | −13.413 | 1.00 | 18.24 | A |
| ATOM | 3478 | CD2 | HIS | A | 446 | 71.044 | 61.370 | −14.047 | 1.00 | 19.05 | A |
| ATOM | 3479 | ND1 | HIS | A | 446 | 69.047 | 62.201 | −14.350 | 1.00 | 18.92 | A |
| ATOM | 3480 | CE1 | HIS | A | 446 | 69.688 | 62.296 | −15.501 | 1.00 | 19.41 | A |
| ATOM | 3481 | NE2 | HIS | A | 446 | 70.901 | 61.799 | −15.345 | 1.00 | 19.48 | A |
| ATOM | 3482 | C | HIS | A | 446 | 68.711 | 59.942 | −10.201 | 1.00 | 16.41 | A |
| ATOM | 3483 | O | HIS | A | 446 | 67.625 | 60.071 | −9.635 | 1.00 | 15.17 | A |
| ATOM | 3484 | N | SER | A | 447 | 69.855 | 59.714 | −9.566 | 1.00 | 16.74 | A |
| ATOM | 3485 | CA | SER | A | 447 | 69.946 | 59.673 | −8.120 | 1.00 | 17.65 | A |
| ATOM | 3486 | CB | SER | A | 447 | 70.995 | 58.647 | −7.685 | 1.00 | 18.93 | A |
| ATOM | 3487 | OG | SER | A | 447 | 70.995 | 58.488 | −6.280 | 1.00 | 20.59 | A |
| ATOM | 3488 | C | SER | A | 447 | 70.432 | 61.099 | −7.850 | 1.00 | 17.81 | A |
| ATOM | 3489 | O | SER | A | 447 | 71.333 | 61.583 | −8.538 | 1.00 | 18.00 | A |
| ATOM | 3490 | N | TRP | A | 448 | 69.832 | 61.789 | −6.886 | 1.00 | 17.24 | A |
| ATOM | 3491 | CA | TRP | A | 448 | 70.232 | 63.165 | −6.619 | 1.00 | 17.67 | A |
| ATOM | 3492 | CB | TRP | A | 448 | 69.020 | 64.097 | −6.707 | 1.00 | 16.94 | A |
| ATOM | 3493 | CG | TRP | A | 448 | 68.372 | 64.127 | −8.058 | 1.00 | 15.88 | A |
| ATOM | 3494 | CD2 | TRP | A | 448 | 68.520 | 65.144 | −9.056 | 1.00 | 15.36 | A |
| ATOM | 3495 | CE2 | TRP | A | 448 | 67.742 | 64.752 | −10.168 | 1.00 | 14.69 | A |
| ATOM | 3496 | CE3 | TRP | A | 448 | 69.236 | 66.350 | −9.120 | 1.00 | 14.92 | A |
| ATOM | 3497 | CD1 | TRP | A | 448 | 67.537 | 63.190 | −8.587 | 1.00 | 15.27 | A |
| ATOM | 3498 | NE1 | TRP | A | 448 | 67.154 | 63.555 | −9.854 | 1.00 | 14.78 | A |
| ATOM | 3499 | CZ2 | TRP | A | 448 | 67.659 | 65.520 | −11.332 | 1.00 | 14.71 | A |
| ATOM | 3500 | CZ3 | TRP | A | 448 | 69.154 | 67.116 | −10.278 | 1.00 | 15.23 | A |
| ATOM | 3501 | CH2 | TRP | A | 448 | 68.368 | 66.696 | −11.370 | 1.00 | 14.57 | A |
| ATOM | 3502 | C | TRP | A | 448 | 70.919 | 63.392 | −5.283 | 1.00 | 18.42 | A |
| ATOM | 3503 | O | TRP | A | 448 | 70.585 | 62.760 | −4.284 | 1.00 | 17.49 | A |
| ATOM | 3504 | N | ASP | A | 449 | 71.880 | 64.312 | −5.282 | 1.00 | 20.05 | A |
| ATOM | 3505 | CA | ASP | A | 449 | 72.605 | 64.659 | −4.068 | 1.00 | 21.64 | A |
| ATOM | 3506 | CB | ASP | A | 449 | 73.759 | 65.612 | −4.397 | 1.00 | 23.78 | A |
| ATOM | 3507 | CG | ASP | A | 449 | 74.633 | 65.915 | −3.192 | 1.00 | 26.46 | A |
| ATOM | 3508 | OD1 | ASP | A | 449 | 74.187 | 66.658 | −2.290 | 1.00 | 27.14 | A |
| ATOM | 3509 | OD2 | ASP | A | 449 | 75.772 | 65.402 | −3.146 | 1.00 | 28.75 | A |
| ATOM | 3510 | C | ASP | A | 449 | 71.607 | 65.344 | −3.140 | 1.00 | 21.51 | A |
| ATOM | 3511 | O | ASP | A | 449 | 70.723 | 66.066 | −3.599 | 1.00 | 21.20 | A |
| ATOM | 3512 | N | GLY | A | 450 | 71.743 | 65.109 | −1.839 | 1.00 | 21.76 | A |
| ATOM | 3513 | CA | GLY | A | 450 | 70.833 | 65.708 | −0.881 | 1.00 | 21.96 | A |
| ATOM | 3514 | C | GLY | A | 450 | 70.708 | 67.216 | −0.997 | 1.00 | 22.35 | A |
| ATOM | 3515 | O | GLY | A | 450 | 69.662 | 67.782 | −0.677 | 1.00 | 22.04 | A |
| ATOM | 3516 | N | MET | A | 451 | 71.769 | 67.872 | −1.454 | 1.00 | 21.87 | A |
| ATOM | 3517 | CA | MET | A | 451 | 71.756 | 69.323 | −1.593 | 1.00 | 22.85 | A |
| ATOM | 3518 | CB | MET | A | 451 | 73.156 | 69.840 | −1.937 | 1.00 | 25.58 | A |
| ATOM | 3519 | CG | MET | A | 451 | 74.196 | 69.618 | −0.856 | 1.00 | 29.68 | A |
| ATOM | 3520 | SD | MET | A | 451 | 75.742 | 70.474 | −1.245 | 1.00 | 35.87 | A |
| ATOM | 3521 | CE | MET | A | 451 | 76.613 | 69.212 | −2.205 | 1.00 | 33.78 | A |
| ATOM | 3522 | C | MET | A | 451 | 70.773 | 69.820 | −2.649 | 1.00 | 21.28 | A |
| ATOM | 3523 | O | MET | A | 451 | 70.398 | 70.990 | −2.644 | 1.00 | 21.56 | A |
| ATOM | 3524 | N | ALA | A | 452 | 70.366 | 68.937 | −3.554 | 1.00 | 20.19 | A |
| ATOM | 3525 | CA | ALA | A | 452 | 69.434 | 69.314 | −4.614 | 1.00 | 19.23 | A |
| ATOM | 3526 | CB | ALA | A | 452 | 69.452 | 68.267 | −5.724 | 1.00 | 19.32 | A |
| ATOM | 3527 | C | ALA | A | 452 | 68.017 | 69.482 | −4.077 | 1.00 | 18.38 | A |
| ATOM | 3528 | O | ALA | A | 452 | 67.146 | 70.024 | −4.760 | 1.00 | 17.97 | A |
| ATOM | 3529 | N | ARG | A | 453 | 67.795 | 69.004 | −2.856 | 1.00 | 17.50 | A |
| ATOM | 3530 | CA | ARG | A | 453 | 66.488 | 69.096 | −2.207 | 1.00 | 17.01 | A |
| ATOM | 3531 | CB | ARG | A | 453 | 66.180 | 70.559 | −1.870 | 1.00 | 18.00 | A |
| ATOM | 3532 | CG | ARG | A | 453 | 67.249 | 71.225 | −1.012 | 1.00 | 19.55 | A |
| ATOM | 3533 | CD | ARG | A | 453 | 66.942 | 72.698 | −0.774 | 1.00 | 21.42 | A |
| ATOM | 3534 | NE | ARG | A | 453 | 65.716 | 72.891 | −0.005 | 1.00 | 22.14 | A |
| ATOM | 3535 | CZ | ARG | A | 453 | 65.172 | 74.078 | 0.251 | 1.00 | 23.68 | A |
| ATOM | 3536 | NH1 | ARG | A | 453 | 65.745 | 75.187 | −0.202 | 1.00 | 24.14 | A |
| ATOM | 3537 | NH2 | ARG | A | 453 | 64.054 | 74.156 | 0.963 | 1.00 | 23.59 | A |
| ATOM | 3538 | C | ARG | A | 453 | 65.360 | 68.510 | −3.058 | 1.00 | 16.24 | A |
| ATOM | 3539 | O | ARG | A | 453 | 64.227 | 68.987 | −3.016 | 1.00 | 16.48 | A |
| ATOM | 3540 | N | ILE | A | 454 | 65.673 | 67.471 | −3.826 | 1.00 | 15.16 | A |
| ATOM | 3541 | CA | ILE | A | 454 | 64.681 | 66.830 | −4.679 | 1.00 | 14.83 | A |
| ATOM | 3542 | CB | ILE | A | 454 | 65.349 | 65.848 | −5.667 | 1.00 | 15.09 | A |
| ATOM | 3543 | CG2 | ILE | A | 454 | 64.286 | 65.111 | −6.477 | 1.00 | 14.91 | A |
| ATOM | 3544 | CG1 | ILE | A | 454 | 66.312 | 66.611 | −6.587 | 1.00 | 14.75 | A |
| ATOM | 3545 | CD1 | ILE | A | 454 | 65.660 | 67.697 | −7.432 | 1.00 | 14.56 | A |
| ATOM | 3546 | C | ILE | A | 454 | 63.638 | 66.077 | −3.852 | 1.00 | 14.81 | A |
| ATOM | 3547 | O | ILE | A | 454 | 62.438 | 66.309 | −4.002 | 1.00 | 14.37 | A |
| ATOM | 3548 | N | GLU | A | 455 | 64.095 | 65.179 | −2.981 | 1.00 | 14.40 | A |
| ATOM | 3549 | CA | GLU | A | 455 | 63.178 | 64.410 | −2.142 | 1.00 | 14.75 | A |
| ATOM | 3550 | CB | GLU | A | 455 | 63.944 | 63.458 | −1.212 | 1.00 | 15.34 | A |
| ATOM | 3551 | CG | GLU | A | 455 | 64.535 | 62.225 | −1.883 | 1.00 | 14.65 | A |
| ATOM | 3552 | CD | GLU | A | 455 | 65.880 | 62.484 | −2.539 | 1.00 | 16.24 | A |
| ATOM | 3553 | OE1 | GLU | A | 455 | 66.344 | 63.646 | −2.526 | 1.00 | 16.23 | A |
| ATOM | 3554 | OE2 | GLU | A | 455 | 66.473 | 61.518 | −3.068 | 1.00 | 14.90 | A |
| ATOM | 3555 | C | GLU | A | 455 | 62.323 | 65.349 | −1.299 | 1.00 | 14.60 | A |
| ATOM | 3556 | O | GLU | A | 455 | 61.129 | 65.116 | −1.103 | 1.00 | 13.65 | A |
| ATOM | 3557 | N | GLU | A | 456 | 62.948 | 66.411 | −0.800 | 1.00 | 14.87 | A |
| ATOM | 3558 | CA | GLU | A | 456 | 62.258 | 67.394 | 0.025 | 1.00 | 15.38 | A |
| ATOM | 3559 | CB | GLU | A | 456 | 63.229 | 68.502 | 0.432 | 1.00 | 17.41 | A |
| ATOM | 3560 | CG | GLU | A | 456 | 62.669 | 69.473 | 1.454 | 1.00 | 20.04 | A |
| ATOM | 3561 | CD | GLU | A | 456 | 63.543 | 70.699 | 1.627 | 1.00 | 21.34 | A |
| ATOM | 3562 | OE1 | GLU | A | 456 | 64.773 | 70.581 | 1.457 | 1.00 | 22.77 | A |
| ATOM | 3563 | OE2 | GLU | A | 456 | 63.002 | 71.780 | 1.941 | 1.00 | 22.99 | A |
| ATOM | 3564 | C | GLU | A | 456 | 61.070 | 68.011 | −0.713 | 1.00 | 14.91 | A |
| ATOM | 3565 | O | GLU | A | 456 | 59.940 | 67.995 | −0.222 | 1.00 | 14.66 | A |
| ATOM | 3566 | N | ARG | A | 457 | 61.333 | 68.563 | −1.893 | 1.00 | 14.02 | A |
| ATOM | 3567 | CA | ARG | A | 457 | 60.281 | 69.196 | −2.686 | 1.00 | 13.76 | A |
| ATOM | 3568 | CB | ARG | A | 457 | 60.895 | 69.878 | −3.917 | 1.00 | 14.11 | A |
| ATOM | 3569 | CG | ARG | A | 457 | 61.308 | 71.340 | −3.700 | 1.00 | 15.77 | A |
| ATOM | 3570 | CD | ARG | A | 457 | 62.227 | 71.524 | −2.493 | 1.00 | 18.41 | A |
| ATOM | 3571 | NE | ARG | A | 457 | 62.485 | 72.936 | −2.197 | 1.00 | 19.24 | A |
| ATOM | 3572 | CZ | ARG | A | 457 | 63.341 | 73.709 | −2.864 | 1.00 | 20.83 | A |
| ATOM | 3573 | NH1 | ARG | A | 457 | 64.041 | 73.217 | −3.879 | 1.00 | 20.68 | A |
| ATOM | 3574 | NH2 | ARG | A | 457 | 63.497 | 74.982 | −2.515 | 1.00 | 21.40 | A |
| ATOM | 3575 | C | ARG | A | 457 | 59.186 | 68.216 | −3.110 | 1.00 | 12.75 | A |
| ATOM | 3576 | O | ARG | A | 457 | 58.004 | 68.561 | −3.105 | 1.00 | 11.81 | A |
| ATOM | 3577 | N | LEU | A | 458 | 59.571 | 66.996 | −3.470 | 1.00 | 11.91 | A |
| ATOM | 3578 | CA | LEU | A | 458 | 58.586 | 66.002 | −3.886 | 1.00 | 12.47 | A |
| ATOM | 3579 | CB | LEU | A | 458 | 59.279 | 64.788 | −4.511 | 1.00 | 12.02 | A |
| ATOM | 3580 | CG | LEU | A | 458 | 59.998 | 65.089 | −5.834 | 1.00 | 11.94 | A |
| ATOM | 3581 | CD1 | LEU | A | 458 | 60.693 | 63.829 | −6.341 | 1.00 | 11.69 | A |
| ATOM | 3582 | CD2 | LEU | A | 458 | 58.985 | 65.601 | −6.866 | 1.00 | 11.22 | A |
| ATOM | 3583 | C | LEU | A | 458 | 57.696 | 65.559 | −2.727 | 1.00 | 12.74 | A |
| ATOM | 3584 | O | LEU | A | 458 | 56.505 | 65.318 | −2.917 | 1.00 | 12.98 | A |
| ATOM | 3585 | N | GLU | A | 459 | 58.265 | 65.451 | −1.530 | 1.00 | 12.29 | A |
| ATOM | 3586 | CA | GLU | A | 459 | 57.473 | 65.049 | −0.374 | 1.00 | 13.18 | A |
| ATOM | 3587 | CB | GLU | A | 459 | 58.365 | 64.846 | 0.857 | 1.00 | 14.23 | A |
| ATOM | 3588 | CG | GLU | A | 459 | 57.595 | 64.361 | 2.088 | 1.00 | 16.92 | A |
| ATOM | 3589 | CD | GLU | A | 459 | 58.497 | 63.766 | 3.159 | 1.00 | 18.01 | A |
| ATOM | 3590 | OE1 | GLU | A | 459 | 59.210 | 64.532 | 3.834 | 1.00 | 19.27 | A |
| ATOM | 3591 | OE2 | GLU | A | 459 | 58.497 | 62.525 | 3.316 | 1.00 | 18.89 | A |
| ATOM | 3592 | C | GLU | A | 459 | 56.428 | 66.126 | −0.096 | 1.00 | 12.90 | A |
| ATOM | 3593 | O | GLU | A | 459 | 55.259 | 65.824 | 0.153 | 1.00 | 11.83 | A |
| ATOM | 3594 | N | GLN | A | 460 | 56.847 | 67.388 | −0.147 | 1.00 | 12.61 | A |
| ATOM | 3595 | CA | GLN | A | 460 | 55.921 | 68.490 | 0.081 | 1.00 | 13.10 | A |
| ATOM | 3596 | CB | GLN | A | 460 | 56.653 | 69.829 | −0.041 | 1.00 | 15.39 | A |
| ATOM | 3597 | CG | GLN | A | 460 | 55.765 | 71.037 | 0.211 | 1.00 | 17.90 | A |
| ATOM | 3598 | CD | GLN | A | 460 | 56.432 | 72.346 | −0.153 | 1.00 | 20.12 | A |
| ATOM | 3599 | OE1 | GLN | A | 460 | 55.906 | 73.421 | 0.135 | 1.00 | 23.01 | A |
| ATOM | 3600 | NE2 | GLN | A | 460 | 57.590 | 72.265 | −0.799 | 1.00 | 20.79 | A |
| ATOM | 3601 | C | GLN | A | 460 | 54.788 | 68.428 | −0.950 | 1.00 | 12.41 | A |
| ATOM | 3602 | O | GLN | A | 460 | 53.612 | 68.505 | −0.602 | 1.00 | 11.47 | A |
| ATOM | 3603 | N | ALA | A | 461 | 55.145 | 68.278 | −2.222 | 1.00 | 11.44 | A |
| ATOM | 3604 | CA | ALA | A | 461 | 54.136 | 68.217 | −3.275 | 1.00 | 10.82 | A |
| ATOM | 3605 | CB | ALA | A | 461 | 54.810 | 68.096 | −4.646 | 1.00 | 10.93 | A |
| ATOM | 3606 | C | ALA | A | 461 | 53.147 | 67.069 | −3.068 | 1.00 | 10.35 | A |
| ATOM | 3607 | O | ALA | A | 461 | 51.935 | 67.270 | −3.152 | 1.00 | 10.11 | A |
| ATOM | 3608 | N | ARG | A | 462 | 53.659 | 65.869 | −2.801 | 1.00 | 10.22 | A |
| ATOM | 3609 | CA | ARG | A | 462 | 52.797 | 64.708 | −2.587 | 1.00 | 10.59 | A |
| ATOM | 3610 | CB | ARG | A | 462 | 53.630 | 63.443 | −2.331 | 1.00 | 9.97 | A |
| ATOM | 3611 | CG | ARG | A | 462 | 54.383 | 62.883 | −3.541 | 1.00 | 9.98 | A |
| ATOM | 3612 | CD | ARG | A | 462 | 54.835 | 61.446 | −3.254 | 1.00 | 10.20 | A |
| ATOM | 3613 | NE | ARG | A | 462 | 55.747 | 61.380 | −2.113 | 1.00 | 10.56 | A |
| ATOM | 3614 | CZ | ARG | A | 462 | 57.066 | 61.530 | −2.202 | 1.00 | 11.65 | A |
| ATOM | 3615 | NH1 | ARG | A | 462 | 57.635 | 61.749 | −3.383 | 1.00 | 11.19 | A |
| ATOM | 3616 | NH2 | ARG | A | 462 | 57.818 | 61.469 | −1.110 | 1.00 | 11.26 | A |
| ATOM | 3617 | C | ARG | A | 462 | 51.860 | 64.925 | −1.400 | 1.00 | 11.75 | A |
| ATOM | 3618 | O | ARG | A | 462 | 50.680 | 64.581 | −1.451 | 1.00 | 10.88 | A |
| ATOM | 3619 | N | ARG | A | 463 | 52.390 | 65.498 | −0.325 | 1.00 | 11.61 | A |
| ATOM | 3620 | CA | ARG | A | 463 | 51.582 | 65.721 | 0.863 | 1.00 | 11.89 | A |
| ATOM | 3621 | CB | ARG | A | 463 | 52.498 | 66.003 | 2.056 | 1.00 | 11.99 | A |
| ATOM | 3622 | CG | ARG | A | 463 | 53.276 | 64.751 | 2.436 | 1.00 | 12.82 | A |
| ATOM | 3623 | CD | ARG | A | 463 | 54.199 | 64.923 | 3.623 | 1.00 | 14.07 | A |
| ATOM | 3624 | NE | ARG | A | 463 | 54.724 | 63.620 | 4.023 | 1.00 | 15.02 | A |
| ATOM | 3625 | CZ | ARG | A | 463 | 55.481 | 63.409 | 5.094 | 1.00 | 15.38 | A |
| ATOM | 3626 | NH1 | ARG | A | 463 | 55.814 | 64.422 | 5.882 | 1.00 | 15.01 | A |
| ATOM | 3627 | NH2 | ARG | A | 463 | 55.893 | 62.180 | 5.381 | 1.00 | 15.17 | A |
| ATOM | 3628 | C | ARG | A | 463 | 50.497 | 66.787 | 0.730 | 1.00 | 11.52 | A |
| ATOM | 3629 | O | ARG | A | 463 | 49.401 | 66.612 | 1.259 | 1.00 | 11.19 | A |
| ATOM | 3630 | N | GLU | A | 464 | 50.777 | 67.881 | 0.024 | 1.00 | 11.08 | A |
| ATOM | 3631 | CA | GLU | A | 464 | 49.755 | 68.913 | −0.137 | 1.00 | 11.07 | A |
| ATOM | 3632 | CB | GLU | A | 464 | 50.361 | 70.206 | −0.686 | 1.00 | 12.30 | A |
| ATOM | 3633 | CG | GLU | A | 464 | 51.509 | 70.752 | 0.158 | 1.00 | 14.44 | A |
| ATOM | 3634 | CD | GLU | A | 464 | 51.083 | 71.173 | 1.560 | 1.00 | 15.33 | A |
| ATOM | 3635 | OE1 | GLU | A | 464 | 50.043 | 70.696 | 2.058 | 1.00 | 16.27 | A |
| ATOM | 3636 | OE2 | GLU | A | 464 | 51.804 | 71.981 | 2.176 | 1.00 | 17.15 | A |
| ATOM | 3637 | C | GLU | A | 464 | 46.653 | 68.409 | −1.067 | 1.00 | 10.16 | A |
| ATOM | 3638 | O | GLU | A | 464 | 47.481 | 68.714 | −0.861 | 1.00 | 10.20 | A |
| ATOM | 3639 | N | LEU | A | 465 | 49.022 | 67.646 | −2.094 | 1.00 | 10.06 | A |
| ATOM | 3640 | CA | LEU | A | 465 | 48.017 | 67.101 | −3.005 | 1.00 | 10.13 | A |
| ATOM | 3641 | CB | LEU | A | 465 | 48.668 | 66.476 | −4.247 | 1.00 | 9.70 | A |
| ATOM | 3642 | CG | LEU | A | 465 | 47.695 | 65.782 | −5.215 | 1.00 | 9.35 | A |
| ATOM | 3643 | CD1 | LEU | A | 465 | 46.662 | 66.790 | −5.716 | 1.00 | 9.18 | A |
| ATOM | 3644 | CD2 | LEU | A | 465 | 48.459 | 65.178 | −6.389 | 1.00 | 8.95 | A |
| ATOM | 3645 | C | LEU | A | 465 | 47.215 | 66.033 | −2.263 | 1.00 | 9.87 | A |
| ATOM | 3646 | O | LEU | A | 465 | 45.994 | 65.955 | −2.400 | 1.00 | 9.64 | A |
| ATOM | 3647 | N | SER | A | 466 | 47.905 | 65.218 | −1.468 | 1.00 | 9.22 | A |
| ATOM | 3648 | CA | SER | A | 466 | 47.237 | 64.161 | −0.713 | 1.00 | 9.23 | A |
| ATOM | 3649 | CB | SER | A | 466 | 48.261 | 63.303 | 0.038 | 1.00 | 9.47 | A |
| ATOM | 3650 | OG | SER | A | 466 | 49.045 | 62.538 | −0.864 | 1.00 | 9.64 | A |
| ATOM | 3651 | C | SER | A | 466 | 46.252 | 64.764 | 0.279 | 1.00 | 9.71 | A |
| ATOM | 3652 | O | SER | A | 466 | 45.148 | 64.249 | 0.467 | 1.00 | 9.34 | A |
| ATOM | 3653 | N | LEU | A | 467 | 46.656 | 65.861 | 0.910 | 1.00 | 9.09 | A |
| ATOM | 3654 | CA | LEU | A | 467 | 45.795 | 66.521 | 1.879 | 1.00 | 9.59 | A |
| ATOM | 3655 | CB | LEU | A | 467 | 46.501 | 67.744 | 2.473 | 1.00 | 9.34 | A |
| ATOM | 3656 | CG | LEU | A | 467 | 45.771 | 68.418 | 3.636 | 1.00 | 11.49 | A |
| ATOM | 3657 | CD1 | LEU | A | 467 | 45.861 | 67.523 | 4.867 | 1.00 | 12.53 | A |
| ATOM | 3658 | CD2 | LEU | A | 467 | 46.389 | 69.786 | 3.924 | 1.00 | 11.51 | A |
| ATOM | 3659 | C | LEU | A | 467 | 44.480 | 66.957 | 1.233 | 1.00 | 9.10 | A |
| ATOM | 3660 | O | LEU | A | 467 | 43.405 | 66.769 | 1.809 | 1.00 | 8.58 | A |
| ATOM | 3661 | N | PHE | A | 468 | 44.569 | 67.521 | 0.032 | 1.00 | 8.59 | A |
| ATOM | 3662 | CA | PHE | A | 468 | 43.386 | 68.005 | −0.672 | 1.00 | 9.47 | A |
| ATOM | 3663 | CB | PHE | A | 468 | 43.792 | 68.803 | −1.918 | 1.00 | 9.60 | A |
| ATOM | 3664 | CG | PHE | A | 468 | 42.667 | 69.600 | −2.519 | 1.00 | 10.53 | A |
| ATOM | 3665 | CD1 | PHE | A | 468 | 41.953 | 70.514 | −1.744 | 1.00 | 10.08 | A |
| ATOM | 3666 | CD2 | PHE | A | 468 | 42.310 | 69.434 | −3.854 | 1.00 | 9.96 | A |
| ATOM | 3667 | CE1 | PHE | A | 468 | 40.898 | 71.250 | −2.291 | 1.00 | 9.64 | A |
| ATOM | 3668 | CE2 | PHE | A | 468 | 41.256 | 70.166 | −4.409 | 1.00 | 9.46 | A |
| ATOM | 3669 | CZ | PHE | A | 468 | 40.549 | 71.076 | −3.622 | 1.00 | 8.13 | A |
| ATOM | 3670 | C | PHE | A | 468 | 42.405 | 66.901 | −1.060 | 1.00 | 9.54 | A |
| ATOM | 3671 | O | PHE | A | 468 | 41.261 | 67.186 | −1.400 | 1.00 | 10.30 | A |
| ATOM | 3672 | N | GLN | A | 469 | 42.843 | 65.644 | −1.012 | 1.00 | 9.36 | A |
| ATOM | 3673 | CA | GLN | A | 469 | 41.951 | 64.536 | −1.343 | 1.00 | 9.71 | A |
| ATOM | 3674 | CB | GLN | A | 469 | 42.743 | 63.243 | −1.555 | 1.00 | 9.78 | A |
| ATOM | 3675 | CG | GLN | A | 469 | 43.803 | 63.345 | −2.645 | 1.00 | 10.20 | A |
| ATOM | 3676 | CD | GLN | A | 469 | 43.265 | 63.975 | −3.917 | 1.00 | 9.47 | A |
| ATOM | 3677 | OE1 | GLN | A | 469 | 43.797 | 64.979 | −4.397 | 1.00 | 12.79 | A |
| ATOM | 3678 | NE2 | GLN | A | 469 | 42.207 | 63.394 | −4.468 | 1.00 | 8.19 | A |
| ATOM | 3679 | C | GLN | A | 469 | 40.921 | 64.325 | −0.234 | 1.00 | 9.75 | A |
| ATOM | 3680 | O | GLN | A | 469 | 39.977 | 63.545 | −0.392 | 1.00 | 10.11 | A |
| ATOM | 3681 | N | HIS | A | 470 | 41.111 | 65.023 | 0.884 | 1.00 | 9.95 | A |
| ATOM | 3682 | CA | HIS | A | 470 | 40.200 | 64.937 | 2.029 | 1.00 | 9.97 | A |
| ATOM | 3683 | CB | HIS | A | 470 | 40.571 | 65.989 | 3.082 | 1.00 | 10.40 | A |
| ATOM | 3684 | CG | HIS | A | 470 | 39.592 | 66.076 | 4.213 | 1.00 | 11.67 | A |
| ATOM | 3685 | CD2 | HIS | A | 470 | 38.969 | 65.109 | 4.926 | 1.00 | 11.04 | A |
| ATOM | 3686 | ND1 | HIS | A | 470 | 39.127 | 67.276 | 4.709 | 1.00 | 13.03 | A |
| ATOM | 3687 | CE1 | HIS | A | 470 | 38.258 | 67.043 | 5.676 | 1.00 | 10.74 | A |
| ATOM | 3688 | NE2 | HIS | A | 470 | 38.144 | 65.736 | 5.828 | 1.00 | 13.35 | A |
| ATOM | 3689 | C | HIS | A | 470 | 38.754 | 65.165 | 1.583 | 1.00 | 10.37 | A |
| ATOM | 3690 | O | HIS | A | 470 | 38.511 | 65.900 | 0.622 | 1.00 | 9.96 | A |
| ATOM | 3691 | N | HIS | A | 471 | 37.797 | 64.560 | 2.289 | 1.00 | 10.77 | A |
| ATOM | 3692 | CA | HIS | A | 471 | 36.391 | 64.709 | 1.923 | 1.00 | 11.23 | A |
| ATOM | 3693 | CB | HIS | A | 471 | 35.525 | 63.621 | 2.593 | 1.00 | 11.19 | A |
| ATOM | 3694 | CG | HIS | A | 471 | 35.686 | 63.532 | 4.078 | 1.00 | 10.36 | A |
| ATOM | 3695 | CD2 | HIS | A | 471 | 35.001 | 64.122 | 5.086 | 1.00 | 10.59 | A |
| ATOM | 3696 | ND1 | HIS | A | 471 | 36.646 | 62.744 | 4.678 | 1.00 | 10.71 | A |
| ATOM | 3697 | CE1 | HIS | A | 471 | 36.545 | 62.853 | 5.990 | 1.00 | 10.31 | A |
| ATOM | 3698 | NE2 | HIS | A | 471 | 35.555 | 63.683 | 6.263 | 1.00 | 9.96 | A |
| ATOM | 3699 | C | HIS | A | 471 | 35.773 | 66.099 | 2.147 | 1.00 | 11.46 | A |
| ATOM | 3700 | O | HIS | A | 471 | 34.555 | 66.270 | 2.012 | 1.00 | 10.89 | A |
| ATOM | 3701 | N | ASP | A | 472 | 36.606 | 67.080 | 2.502 | 1.00 | 11.51 | A |
| ATOM | 3702 | CA | ASP | A | 472 | 36.163 | 68.467 | 2.651 | 1.00 | 11.16 | A |
| ATOM | 3703 | CB | ASP | A | 472 | 36.184 | 68.929 | 4.108 | 1.00 | 12.11 | A |
| ATOM | 3704 | CG | ASP | A | 472 | 35.075 | 68.316 | 4.919 | 1.00 | 11.49 | A |
| ATOM | 3705 | OD1 | ASP | A | 472 | 33.913 | 68.402 | 4.475 | 1.00 | 12.78 | A |
| ATOM | 3706 | OD2 | ASP | A | 472 | 35.363 | 67.755 | 5.991 | 1.00 | 12.45 | A |
| ATOM | 3707 | C | ASP | A | 472 | 37.112 | 69.337 | 1.834 | 1.00 | 11.58 | A |
| ATOM | 3708 | O | ASP | A | 472 | 37.043 | 70.568 | 1.870 | 1.00 | 11.63 | A |
| ATOM | 3709 | N | GLY | A | 473 | 38.005 | 68.679 | 1.103 | 1.00 | 10.90 | A |
| ATOM | 3710 | CA | GLY | A | 473 | 38.956 | 69.394 | 0.275 | 1.00 | 10.57 | A |
| ATOM | 3711 | C | GLY | A | 473 | 38.445 | 69.511 | −1.145 | 1.00 | 10.47 | A |
| ATOM | 3712 | O | GLY | A | 473 | 37.745 | 70.464 | −1.483 | 1.00 | 10.79 | A |
| ATOM | 3713 | N | ILE | A | 474 | 38.783 | 68.529 | −1.976 | 1.00 | 10.32 | A |
| ATOM | 3714 | CA | ILE | A | 474 | 38.368 | 68.518 | −3.375 | 1.00 | 10.66 | A |
| ATOM | 3715 | CB | ILE | A | 474 | 38.904 | 67.239 | −4.090 | 1.00 | 11.01 | A |
| ATOM | 3716 | CG2 | ILE | A | 474 | 38.293 | 65.985 | −3.462 | 1.00 | 10.23 | A |
| ATOM | 3717 | CG1 | ILE | A | 474 | 38.614 | 67.314 | −5.591 | 1.00 | 11.39 | A |
| ATOM | 3718 | CD1 | ILE | A | 474 | 39.203 | 66.158 | −6.393 | 1.00 | 11.29 | A |
| ATOM | 3719 | C | ILE | A | 474 | 36.844 | 68.618 | −3.536 | 1.00 | 10.82 | A |
| ATOM | 3720 | O | ILE | A | 474 | 36.344 | 69.060 | −4.573 | 1.00 | 10.27 | A |
| ATOM | 3721 | N | THR | A | 475 | 36.119 | 68.220 | −2.495 | 1.00 | 10.66 | A |
| ATOM | 3722 | CA | THR | A | 475 | 34.656 | 68.256 | −2.483 | 1.00 | 11.04 | A |
| ATOM | 3723 | CB | THR | A | 475 | 34.108 | 67.616 | −1.205 | 1.00 | 10.92 | A |
| ATOM | 3724 | OG1 | THR | A | 475 | 34.649 | 68.312 | −0.075 | 1.00 | 10.89 | A |
| ATOM | 3725 | CG2 | THR | A | 475 | 34.493 | 66.133 | −1.123 | 1.00 | 9.32 | A |
| ATOM | 3726 | C | THR | A | 475 | 34.107 | 69.684 | −2.520 | 1.00 | 11.15 | A |
| ATOM | 3727 | O | THR | A | 475 | 32.939 | 69.899 | −2.862 | 1.00 | 11.97 | A |
| ATOM | 3728 | N | GLY | A | 476 | 34.940 | 70.649 | −2.146 | 1.00 | 10.97 | A |
| ATOM | 3729 | CA | GLY | A | 476 | 34.500 | 72.034 | −2.124 | 1.00 | 11.23 | A |
| ATOM | 3730 | C | GLY | A | 476 | 33.508 | 72.291 | −0.999 | 1.00 | 11.45 | A |
| ATOM | 3731 | O | GLY | A | 476 | 32.622 | 73.137 | −1.127 | 1.00 | 11.61 | A |
| ATOM | 3732 | N | THR | A | 477 | 33.656 | 71.569 | 0.110 | 1.00 | 11.28 | A |
| ATOM | 3733 | CA | THR | A | 477 | 32.748 | 71.728 | 1.241 | 1.00 | 11.26 | A |
| ATOM | 3734 | CB | THR | A | 477 | 32.089 | 70.369 | 1.618 | 1.00 | 11.46 | A |
| ATOM | 3735 | OG1 | THR | A | 477 | 33.106 | 69.399 | 1.905 | 1.00 | 10.63 | A |
| ATOM | 3736 | CG2 | THR | A | 477 | 31.223 | 69.864 | 0.468 | 1.00 | 11.11 | A |
| ATOM | 3737 | C | THR | A | 477 | 33.370 | 72.333 | 2.502 | 1.00 | 12.05 | A |
| ATOM | 3738 | O | THR | A | 477 | 32.849 | 72.140 | 3.601 | 1.00 | 12.10 | A |
| ATOM | 3739 | N | ALA | A | 478 | 34.469 | 73.072 | 2.354 | 1.00 | 12.04 | A |
| ATOM | 3740 | CA | ALA | A | 478 | 35.116 | 73.694 | 3.512 | 1.00 | 12.48 | A |
| ATOM | 3741 | CB | ALA | A | 478 | 36.632 | 73.485 | 3.445 | 1.00 | 11.99 | A |
| ATOM | 3742 | C | ALA | A | 478 | 34.800 | 75.188 | 3.589 | 1.00 | 12.76 | A |
| ATOM | 3743 | O | ALA | A | 478 | 34.332 | 75.780 | 2.620 | 1.00 | 12.65 | A |
| ATOM | 3744 | N | LYS | A | 479 | 35.049 | 75.805 | 4.740 | 1.00 | 13.47 | A |
| ATOM | 3745 | CA | LYS | A | 479 | 34.783 | 77.235 | 4.860 | 1.00 | 13.72 | A |
| ATOM | 3746 | CB | LYS | A | 479 | 34.926 | 77.699 | 6.316 | 1.00 | 15.00 | A |
| ATOM | 3747 | CG | LYS | A | 479 | 33.901 | 77.059 | 7.254 | 1.00 | 17.01 | A |
| ATOM | 3748 | CD | LYS | A | 479 | 33.941 | 77.653 | 8.660 | 1.00 | 18.59 | A |
| ATOM | 3749 | CE | LYS | A | 479 | 33.028 | 78.861 | 8.808 | 1.00 | 19.93 | A |
| ATOM | 3750 | NZ | LYS | A | 479 | 31.570 | 78.514 | 8.789 | 1.00 | 18.25 | A |
| ATOM | 3751 | C | LYS | A | 479 | 35.761 | 77.984 | 3.963 | 1.00 | 13.63 | A |
| ATOM | 3752 | O | LYS | A | 479 | 36.827 | 77.471 | 3.624 | 1.00 | 12.30 | A |
| ATOM | 3753 | N | THR | A | 480 | 35.390 | 79.202 | 3.591 | 1.00 | 13.88 | A |
| ATOM | 3754 | CA | THR | A | 480 | 36.197 | 80.038 | 2.713 | 1.00 | 13.85 | A |
| ATOM | 3755 | CB | THR | A | 480 | 35.608 | 81.462 | 2.643 | 1.00 | 14.71 | A |
| ATOM | 3756 | OG1 | THR | A | 480 | 34.264 | 81.386 | 2.162 | 1.00 | 15.03 | A |
| ATOM | 3757 | CG2 | THR | A | 480 | 36.430 | 82.352 | 1.701 | 1.00 | 15.64 | A |
| ATOM | 3758 | C | THR | A | 480 | 37.687 | 80.141 | 3.033 | 1.00 | 13.99 | A |
| ATOM | 3759 | O | THR | A | 480 | 38.516 | 79.981 | 2.138 | 1.00 | 13.75 | A |
| ATOM | 3760 | N | HIS | A | 481 | 38.039 | 80.400 | 4.290 | 1.00 | 13.63 | A |
| ATOM | 3761 | CA | HIS | A | 481 | 39.452 | 80.537 | 4.628 | 1.00 | 13.00 | A |
| ATOM | 3762 | CB | HIS | A | 481 | 39.633 | 81.227 | 5.994 | 1.00 | 14.49 | A |
| ATOM | 3763 | CG | HIS | A | 481 | 39.486 | 80.319 | 7.176 | 1.00 | 15.14 | A |
| ATOM | 3764 | CD2 | HIS | A | 481 | 40.412 | 79.799 | 8.016 | 1.00 | 15.70 | A |
| ATOM | 3765 | ND1 | HIS | A | 481 | 38.264 | 79.867 | 7.626 | 1.00 | 15.87 | A |
| ATOM | 3766 | CE1 | HIS | A | 481 | 38.445 | 79.109 | 8.693 | 1.00 | 16.85 | A |
| ATOM | 3767 | NE2 | HIS | A | 481 | 39.739 | 79.051 | 8.949 | 1.00 | 15.85 | A |
| ATOM | 3768 | C | HIS | A | 481 | 40.205 | 79.211 | 4.588 | 1.00 | 12.69 | A |
| ATOM | 3769 | O | HIS | A | 481 | 41.429 | 79.194 | 4.445 | 1.00 | 11.59 | A |
| ATOM | 3770 | N | VAL | A | 482 | 39.474 | 78.104 | 4.698 | 1.00 | 11.97 | A |
| ATOM | 3771 | CA | VAL | A | 482 | 40.092 | 76.775 | 4.644 | 1.00 | 12.15 | A |
| ATOM | 3772 | CB | VAL | A | 482 | 39.162 | 75.704 | 5.261 | 1.00 | 11.50 | A |
| ATOM | 3773 | CG1 | VAL | A | 482 | 39.836 | 74.327 | 5.235 | 1.00 | 9.91 | A |
| ATOM | 3774 | CG2 | VAL | A | 482 | 38.828 | 76.093 | 6.699 | 1.00 | 12.67 | A |
| ATOM | 3775 | C | VAL | A | 482 | 40.381 | 76.450 | 3.176 | 1.00 | 12.24 | A |
| ATOM | 3776 | O | VAL | A | 482 | 41.426 | 75.892 | 2.844 | 1.00 | 12.15 | A |
| ATOM | 3777 | N | VAL | A | 483 | 39.451 | 76.809 | 2.297 | 1.00 | 12.27 | A |
| ATOM | 3778 | CA | VAL | A | 483 | 39.641 | 76.592 | 0.867 | 1.00 | 12.33 | A |
| ATOM | 3779 | CB | VAL | A | 483 | 38.426 | 77.111 | 0.063 | 1.00 | 12.56 | A |
| ATOM | 3780 | CG1 | VAL | A | 483 | 38.709 | 77.032 | −1.432 | 1.00 | 13.36 | A |
| ATOM | 3781 | CG2 | VAL | A | 483 | 37.187 | 76.282 | 0.416 | 1.00 | 12.27 | A |
| ATOM | 3782 | C | VAL | A | 483 | 40.892 | 77.366 | 0.447 | 1.00 | 13.46 | A |
| ATOM | 3783 | O | VAL | A | 483 | 41.699 | 76.890 | −0.353 | 1.00 | 13.10 | A |
| ATOM | 3784 | N | VAL | A | 484 | 41.053 | 78.562 | 1.004 | 1.00 | 13.68 | A |
| ATOM | 3785 | CA | VAL | A | 484 | 42.215 | 79.382 | 0.696 | 1.00 | 14.31 | A |
| ATOM | 3786 | CB | VAL | A | 484 | 42.125 | 80.762 | 1.375 | 1.00 | 14.14 | A |
| ATOM | 3787 | CG1 | VAL | A | 484 | 43.458 | 81.499 | 1.254 | 1.00 | 14.60 | A |
| ATOM | 3788 | CG2 | VAL | A | 484 | 41.016 | 81.573 | 0.726 | 1.00 | 14.59 | A |
| ATOM | 3789 | C | VAL | A | 484 | 43.487 | 78.674 | 1.144 | 1.00 | 14.08 | A |
| ATOM | 3790 | O | VAL | A | 484 | 44.489 | 78.683 | 0.430 | 1.00 | 14.29 | A |
| ATOM | 3791 | N | ASP | A | 485 | 43.447 | 78.052 | 2.320 | 1.00 | 13.62 | A |
| ATOM | 3792 | CA | ASP | A | 485 | 44.614 | 77.332 | 2.819 | 1.00 | 13.11 | A |
| ATOM | 3793 | CB | ASP | A | 485 | 44.349 | 76.759 | 4.213 | 1.00 | 13.58 | A |
| ATOM | 3794 | CG | ASP | A | 485 | 45.597 | 76.167 | 4.841 | 1.00 | 14.32 | A |
| ATOM | 3795 | OD1 | ASP | A | 485 | 45.547 | 75.015 | 5.317 | 1.00 | 13.49 | A |
| ATOM | 3796 | OD2 | ASP | A | 485 | 46.636 | 76.861 | 4.858 | 1.00 | 15.48 | A |
| ATOM | 3797 | C | ASP | A | 485 | 44.987 | 76.189 | 1.870 | 1.00 | 12.92 | A |
| ATOM | 3798 | O | ASP | A | 485 | 46.162 | 75.995 | 1.549 | 1.00 | 12.22 | A |
| ATOM | 3799 | N | TYR | A | 486 | 43.990 | 75.426 | 1.428 | 1.00 | 12.46 | A |
| ATOM | 3800 | CA | TYR | A | 486 | 44.247 | 74.312 | 0.512 | 1.00 | 11.89 | A |
| ATOM | 3801 | CB | TYR | A | 486 | 42.949 | 73.574 | 0.173 | 1.00 | 12.04 | A |
| ATOM | 3802 | CG | TYR | A | 486 | 42.350 | 72.740 | 1.292 | 1.00 | 12.07 | A |
| ATOM | 3803 | CD1 | TYR | A | 486 | 40.969 | 72.713 | 1.490 | 1.00 | 12.29 | A |
| ATOM | 3804 | CE1 | TYR | A | 486 | 40.385 | 71.892 | 2.465 | 1.00 | 12.35 | A |
| ATOM | 3805 | CD2 | TYR | A | 486 | 43.147 | 71.922 | 2.104 | 1.00 | 12.28 | A |
| ATOM | 3806 | CE2 | TYR | A | 486 | 42.569 | 71.091 | 3.086 | 1.00 | 12.05 | A |
| ATOM | 3807 | CZ | TYR | A | 486 | 41.185 | 71.086 | 3.253 | 1.00 | 11.64 | A |
| ATOM | 3808 | OH | TYR | A | 486 | 40.587 | 70.270 | 4.193 | 1.00 | 12.66 | A |
| ATOM | 3809 | C | TYR | A | 486 | 44.869 | 74.836 | −0.781 | 1.00 | 12.53 | A |
| ATOM | 3810 | O | TYR | A | 486 | 45.795 | 74.229 | −1.317 | 1.00 | 11.35 | A |
| ATOM | 3811 | N | GLU | A | 487 | 44.353 | 75.960 | −1.278 | 1.00 | 13.10 | A |
| ATOM | 3812 | CA | GLU | A | 487 | 44.861 | 76.552 | −2.513 | 1.00 | 14.38 | A |
| ATOM | 3813 | CB | GLU | A | 487 | 44.027 | 77.771 | −2.929 | 1.00 | 15.28 | A |
| ATOM | 3814 | CG | GLU | A | 487 | 44.305 | 78.207 | −4.367 | 1.00 | 16.75 | A |
| ATOM | 3815 | CD | GLU | A | 487 | 43.592 | 79.489 | −4.773 | 1.00 | 18.58 | A |
| ATOM | 3816 | OE1 | GLU | A | 487 | 42.417 | 79.682 | −4.394 | 1.00 | 19.25 | A |
| ATOM | 3817 | OE2 | GLU | A | 487 | 44.210 | 80.299 | −5.494 | 1.00 | 19.30 | A |
| ATOM | 3818 | C | GLU | A | 487 | 46.315 | 76.979 | −2.350 | 1.00 | 14.58 | A |
| ATOM | 3819 | O | GLU | A | 487 | 47.145 | 76.732 | −3.223 | 1.00 | 14.02 | A |
| ATOM | 3820 | N | GLN | A | 488 | 46.617 | 77.633 | −1.232 | 1.00 | 14.94 | A |
| ATOM | 3821 | CA | GLN | A | 488 | 47.980 | 78.083 | −0.963 | 1.00 | 15.97 | A |
| ATOM | 3822 | CB | GLN | A | 488 | 48.044 | 78.836 | 0.372 | 1.00 | 18.36 | A |
| ATOM | 3823 | CG | GLN | A | 488 | 47.367 | 80.199 | 0.341 | 1.00 | 23.16 | A |
| ATOM | 3824 | CD | GLN | A | 488 | 47.381 | 80.895 | 1.691 | 1.00 | 25.38 | A |
| ATOM | 3825 | OE1 | GLN | A | 488 | 47.039 | 82.072 | 1.794 | 1.00 | 28.24 | A |
| ATOM | 3826 | NE2 | GLN | A | 488 | 47.771 | 80.169 | 2.734 | 1.00 | 27.66 | A |
| ATOM | 3827 | C | GLN | A | 488 | 48.923 | 76.888 | −0.924 | 1.00 | 15.05 | A |
| ATOM | 3828 | O | GLN | A | 488 | 50.023 | 76.938 | −1.474 | 1.00 | 14.75 | A |
| ATOM | 3829 | N | ARG | A | 489 | 48.490 | 75.821 | −0.259 | 1.00 | 13.90 | A |
| ATOM | 3830 | CA | ARG | A | 489 | 49.290 | 74.607 | −0.160 | 1.00 | 13.12 | A |
| ATOM | 3831 | CB | ARG | A | 489 | 48.587 | 73.575 | 0.731 | 1.00 | 13.40 | A |
| ATOM | 3832 | CG | ARG | A | 489 | 48.624 | 73.893 | 2.230 | 1.00 | 14.57 | A |
| ATOM | 3833 | CD | ARG | A | 489 | 47.686 | 72.965 | 3.003 | 1.00 | 16.26 | A |
| ATOM | 3834 | NE | ARG | A | 489 | 47.765 | 73.149 | 4.453 | 1.00 | 16.44 | A |
| ATOM | 3835 | CZ | ARG | A | 489 | 48.672 | 72.572 | 5.238 | 1.00 | 17.26 | A |
| ATOM | 3836 | NH1 | ARG | A | 489 | 49.590 | 71.766 | 4.722 | 1.00 | 16.10 | A |
| ATOM | 3837 | NH2 | ARG | A | 489 | 48.661 | 72.803 | 6.547 | 1.00 | 17.00 | A |
| ATOM | 3838 | C | ARG | A | 489 | 49.526 | 74.009 | −1.544 | 1.00 | 12.86 | A |
| ATOM | 3839 | O | ARG | A | 489 | 50.640 | 73.596 | −1.872 | 1.00 | 12.65 | A |
| ATOM | 3840 | N | MET | A | 490 | 48.483 | 73.956 | −2.366 | 1.00 | 11.94 | A |
| ATOM | 3841 | CA | MET | A | 490 | 48.660 | 73.400 | −3.701 | 1.00 | 12.39 | A |
| ATOM | 3842 | CB | MET | A | 490 | 47.304 | 73.153 | −4.376 | 1.00 | 12.12 | A |
| ATOM | 3843 | CG | MET | A | 490 | 46.539 | 71.985 | −3.753 | 1.00 | 12.55 | A |
| ATOM | 3844 | SD | MET | A | 490 | 45.194 | 71.345 | −4.782 | 1.00 | 14.61 | A |
| ATOM | 3845 | CE | MET | A | 490 | 43.883 | 72.493 | −4.346 | 1.00 | 14.40 | A |
| ATOM | 3846 | C | MET | A | 490 | 49.543 | 74.304 | −4.558 | 1.00 | 12.54 | A |
| ATOM | 3847 | O | MET | A | 490 | 50.291 | 73.825 | −5.409 | 1.00 | 11.67 | A |
| ATOM | 3848 | N | GLN | A | 491 | 49.476 | 75.609 | −4.321 | 1.00 | 13.34 | A |
| ATOM | 3849 | CA | GLN | A | 491 | 50.299 | 76.540 | −5.085 | 1.00 | 15.37 | A |
| ATOM | 3850 | CB | GLN | A | 491 | 49.959 | 77.982 | −4.711 | 1.00 | 17.37 | A |
| ATOM | 3851 | CG | GLN | A | 491 | 50.646 | 79.020 | −5.577 | 1.00 | 21.77 | A |
| ATOM | 3852 | CD | GLN | A | 491 | 50.371 | 78.817 | −7.057 | 1.00 | 23.84 | A |
| ATOM | 3853 | OE1 | GLN | A | 491 | 51.076 | 78.066 | −7.738 | 1.00 | 26.59 | A |
| ATOM | 3854 | NE2 | GLN | A | 491 | 49.334 | 79.476 | −7.559 | 1.00 | 25.84 | A |
| ATOM | 3855 | C | GLN | A | 491 | 51.771 | 76.257 | −4.790 | 1.00 | 15.28 | A |
| ATOM | 3856 | O | GLN | A | 491 | 52.610 | 76.245 | −5.696 | 1.00 | 14.45 | A |
| ATOM | 3857 | N | GLU | A | 492 | 52.081 | 76.022 | −3.519 | 1.00 | 15.54 | A |
| ATOM | 3858 | CA | GLU | A | 492 | 53.451 | 75.730 | −3.121 | 1.00 | 16.41 | A |
| ATOM | 3859 | CB | GLU | A | 492 | 53.551 | 75.675 | −1.593 | 1.00 | 18.54 | A |
| ATOM | 3860 | CG | GLU | A | 492 | 52.990 | 76.922 | −0.915 | 1.00 | 23.76 | A |
| ATOM | 3861 | CD | GLU | A | 492 | 52.969 | 76.825 | 0.600 | 1.00 | 26.74 | A |
| ATOM | 3862 | OE1 | GLU | A | 492 | 52.527 | 75.783 | 1.134 | 1.00 | 28.59 | A |
| ATOM | 3863 | OE2 | GLU | A | 492 | 53.380 | 77.801 | 1.261 | 1.00 | 30.17 | A |
| ATOM | 3864 | C | GLU | A | 492 | 53.875 | 74.395 | −3.733 | 1.00 | 15.06 | A |
| ATOM | 3865 | O | GLU | A | 492 | 55.023 | 74.225 | −4.143 | 1.00 | 15.14 | A |
| ATOM | 3866 | N | ALA | A | 493 | 52.940 | 73.451 | −3.797 | 1.00 | 13.84 | A |
| ATOM | 3867 | CA | ALA | A | 493 | 53.223 | 72.137 | −4.374 | 1.00 | 12.38 | A |
| ATOM | 3868 | CB | ALA | A | 493 | 52.017 | 71.213 | −4.194 | 1.00 | 11.33 | A |
| ATOM | 3869 | C | ALA | A | 493 | 53.555 | 72.290 | −5.859 | 1.00 | 12.51 | A |
| ATOM | 3870 | O | ALA | A | 493 | 54.492 | 71.665 | −6.366 | 1.00 | 11.30 | A |
| ATOM | 3871 | N | LEU | A | 494 | 52.783 | 73.118 | −6.558 | 1.00 | 12.26 | A |
| ATOM | 3872 | CA | LEU | A | 494 | 53.024 | 73.341 | −7.981 | 1.00 | 13.51 | A |
| ATOM | 3873 | CB | LEU | A | 494 | 51.970 | 74.295 | −8.563 | 1.00 | 13.59 | A |
| ATOM | 3874 | CG | LEU | A | 494 | 50.574 | 73.687 | −8.745 | 1.00 | 14.46 | A |
| ATOM | 3875 | CD1 | LEU | A | 494 | 49.568 | 74.768 | −9.146 | 1.00 | 14.29 | A |
| ATOM | 3876 | CD2 | LEU | A | 494 | 50.643 | 72.602 | −9.807 | 1.00 | 14.76 | A |
| ATOM | 3877 | C | LEU | A | 494 | 54.425 | 73.904 | −8.202 | 1.00 | 13.48 | A |
| ATOM | 3878 | O | LEU | A | 494 | 55.142 | 73.456 | −9.093 | 1.00 | 13.66 | A |
| ATOM | 3879 | N | LYS | A | 495 | 54.813 | 74.875 | −7.379 | 1.00 | 14.20 | A |
| ATOM | 3880 | CA | LYS | A | 495 | 56.135 | 75.488 | −7.483 | 1.00 | 14.70 | A |
| ATOM | 3881 | CB | LYS | A | 495 | 56.254 | 76.665 | −6.507 | 1.00 | 17.34 | A |
| ATOM | 3882 | CG | LYS | A | 495 | 55.285 | 77.807 | −6.813 | 1.00 | 20.77 | A |
| ATOM | 3883 | CD | LYS | A | 495 | 55.564 | 79.047 | −5.969 | 1.00 | 23.82 | A |
| ATOM | 3884 | CE | LYS | A | 495 | 55.371 | 78.781 | −4.484 | 1.00 | 25.78 | A |
| ATOM | 3885 | NZ | LYS | A | 495 | 55.661 | 79.995 | −3.662 | 1.00 | 28.04 | A |
| ATOM | 3886 | C | LYS | A | 495 | 57.222 | 74.454 | −7.197 | 1.00 | 14.05 | A |
| ATOM | 3887 | O | LYS | A | 495 | 58.270 | 74.449 | −7.847 | 1.00 | 12.58 | A |
| ATOM | 3888 | N | ALA | A | 496 | 56.966 | 73.578 | −6.228 | 1.00 | 12.65 | A |
| ATOM | 3889 | CA | ALA | A | 496 | 57.918 | 72.528 | −5.884 | 1.00 | 12.33 | A |
| ATOM | 3890 | CB | ALA | A | 496 | 57.411 | 71.729 | −4.687 | 1.00 | 12.48 | A |
| ATOM | 3891 | C | ALA | A | 496 | 58.099 | 71.606 | −7.089 | 1.00 | 12.17 | A |
| ATOM | 3892 | O | ALA | A | 496 | 59.220 | 71.230 | −7.437 | 1.00 | 11.21 | A |
| ATOM | 3893 | N | CYS | A | 497 | 56.988 | 71.232 | −7.717 | 1.00 | 11.95 | A |
| ATOM | 3894 | CA | CYS | A | 497 | 57.039 | 70.362 | −8.885 | 1.00 | 11.78 | A |
| ATOM | 3895 | CB | CYS | A | 497 | 55.621 | 70.008 | −9.358 | 1.00 | 11.55 | A |
| ATOM | 3896 | SG | CYS | A | 497 | 54.771 | 68.821 | −8.280 | 1.00 | 11.22 | A |
| ATOM | 3897 | C | CYS | A | 497 | 57.816 | 71.034 | −10.011 | 1.00 | 11.98 | A |
| ATOM | 3898 | O | CYS | A | 497 | 58.656 | 70.404 | −10.650 | 1.00 | 11.59 | A |
| ATOM | 3899 | N | GLN | A | 498 | 57.546 | 72.314 | −10.250 | 1.00 | 12.50 | A |
| ATOM | 3900 | CA | GLN | A | 498 | 58.255 | 73.035 | −11.305 | 1.00 | 13.65 | A |
| ATOM | 3901 | CB | GLN | A | 498 | 57.775 | 74.488 | −11.393 | 1.00 | 15.53 | A |
| ATOM | 3902 | CG | GLN | A | 498 | 58.581 | 75.330 | −12.375 | 1.00 | 18.75 | A |
| ATOM | 3903 | CD | GLN | A | 498 | 58.080 | 76.760 | −12.484 | 1.00 | 20.66 | A |
| ATOM | 3904 | OE1 | GLN | A | 498 | 57.903 | 77.448 | −11.472 | 1.00 | 23.47 | A |
| ATOM | 3905 | NE2 | GLN | A | 498 | 57.861 | 77.220 | −13.712 | 1.00 | 19.47 | A |
| ATOM | 3906 | C | GLN | A | 498 | 59.762 | 73.015 | −11.054 | 1.00 | 13.54 | A |
| ATOM | 3907 | O | GLN | A | 498 | 60.543 | 72.759 | −11.969 | 1.00 | 13.09 | A |
| ATOM | 3908 | N | MET | A | 499 | 60.172 | 73.291 | −9.819 | 1.00 | 12.68 | A |
| ATOM | 3909 | CA | MET | A | 499 | 61.597 | 73.298 | −9.482 | 1.00 | 13.24 | A |
| ATOM | 3910 | CB | MET | A | 499 | 61.786 | 73.636 | −7.998 | 1.00 | 14.75 | A |
| ATOM | 3911 | CG | MET | A | 499 | 63.220 | 73.501 | −7.483 | 1.00 | 17.49 | A |
| ATOM | 3912 | SD | MET | A | 499 | 64.441 | 74.502 | −8.381 | 1.00 | 21.74 | A |
| ATOM | 3913 | CE | MET | A | 499 | 64.082 | 76.122 | −7.740 | 1.00 | 19.60 | A |
| ATOM | 3914 | C | MET | A | 499 | 62.250 | 71.950 | −9.798 | 1.00 | 12.44 | A |
| ATOM | 3915 | O | MET | A | 499 | 63.302 | 71.892 | −10.430 | 1.00 | 11.75 | A |
| ATOM | 3916 | N | VAL | A | 500 | 61.621 | 70.867 | −9.352 | 1.00 | 11.57 | A |
| ATOM | 3917 | CA | VAL | A | 500 | 62.153 | 69.530 | −9.593 | 1.00 | 11.09 | A |
| ATOM | 3918 | CB | VAL | A | 500 | 61.322 | 68.468 | −8.837 | 1.00 | 10.85 | A |
| ATOM | 3919 | CG1 | VAL | A | 500 | 61.748 | 67.067 | −9.247 | 1.00 | 10.52 | A |
| ATOM | 3920 | CG2 | VAL | A | 500 | 61.508 | 68.651 | −7.331 | 1.00 | 11.06 | A |
| ATOM | 3921 | C | VAL | A | 500 | 62.168 | 69.200 | −11.083 | 1.00 | 11.35 | A |
| ATOM | 3922 | O | VAL | A | 500 | 63.152 | 68.680 | −11.606 | 1.00 | 11.29 | A |
| ATOM | 3923 | N | MET | A | 501 | 61.074 | 69.510 | −11.766 | 1.00 | 11.53 | A |
| ATOM | 3924 | CA | MET | A | 501 | 60.976 | 69.232 | −13.192 | 1.00 | 11.54 | A |
| ATOM | 3925 | CB | MET | A | 501 | 59.585 | 69.617 | −13.702 | 1.00 | 12.75 | A |
| ATOM | 3926 | CG | MET | A | 501 | 58.476 | 68.705 | −13.199 | 1.00 | 13.12 | A |
| ATOM | 3927 | SD | MET | A | 501 | 56.834 | 69.365 | −13.536 | 1.00 | 17.72 | A |
| ATOM | 3928 | CE | MET | A | 501 | 56.676 | 69.003 | −15.257 | 1.00 | 16.00 | A |
| ATOM | 3929 | C | MET | A | 501 | 62.048 | 69.951 | −14.006 | 1.00 | 12.05 | A |
| ATOM | 3930 | O | MET | A | 501 | 62.744 | 69.323 | −14.809 | 1.00 | 12.43 | A |
| ATOM | 3931 | N | GLN | A | 502 | 62.189 | 71.259 | −13.798 | 1.00 | 11.82 | A |
| ATOM | 3932 | CA | GLN | A | 502 | 63.172 | 72.032 | −14.553 | 1.00 | 12.11 | A |
| ATOM | 3933 | CB | GLN | A | 502 | 62.967 | 73.534 | −14.315 | 1.00 | 12.25 | A |
| ATOM | 3934 | CG | GLN | A | 502 | 63.209 | 74.023 | −12.898 | 1.00 | 12.56 | A |
| ATOM | 3935 | CD | GLN | A | 502 | 64.600 | 74.614 | −12.725 | 1.00 | 13.84 | A |
| ATOM | 3936 | OE1 | GLN | A | 502 | 65.350 | 74.757 | −13.696 | 1.00 | 13.40 | A |
| ATOM | 3937 | NE2 | GLN | A | 502 | 64.944 | 74.972 | −11.492 | 1.00 | 13.18 | A |
| ATOM | 3938 | C | GLN | A | 502 | 64.612 | 71.609 | −14.252 | 1.00 | 12.59 | A |
| ATOM | 3939 | O | GLN | A | 502 | 65.449 | 71.571 | −15.158 | 1.00 | 11.57 | A |
| ATOM | 3940 | N | GLN | A | 503 | 64.906 | 71.286 | −12.995 | 1.00 | 12.09 | A |
| ATOM | 3941 | CA | GLN | A | 503 | 66.254 | 70.824 | −12.651 | 1.00 | 13.27 | A |
| ATOM | 3942 | CB | GLN | A | 503 | 66.392 | 70.592 | −11.142 | 1.00 | 13.43 | A |
| ATOM | 3943 | CG | GLN | A | 503 | 66.600 | 71.849 | −10.299 | 1.00 | 15.82 | A |
| ATOM | 3944 | CD | GLN | A | 503 | 67.951 | 72.502 | −10.544 | 1.00 | 16.04 | A |
| ATOM | 3945 | OE1 | GLN | A | 503 | 68.944 | 71.820 | −10.810 | 1.00 | 16.22 | A |
| ATOM | 3946 | NE2 | GLN | A | 503 | 67.998 | 73.825 | −10.437 | 1.00 | 17.27 | A |
| ATOM | 3947 | C | GLN | A | 503 | 66.512 | 69.504 | −13.381 | 1.00 | 12.92 | A |
| ATOM | 3948 | O | GLN | A | 503 | 67.598 | 69.276 | −13.911 | 1.00 | 13.19 | A |
| ATOM | 3949 | N | SER | A | 504 | 65.505 | 68.632 | −13.401 | 1.00 | 12.21 | A |
| ATOM | 3950 | CA | SER | A | 504 | 65.629 | 67.338 | −14.070 | 1.00 | 12.69 | A |
| ATOM | 3951 | CB | SER | A | 504 | 64.376 | 66.487 | −13.833 | 1.00 | 12.53 | A |
| ATOM | 3952 | OG | SER | A | 504 | 64.240 | 66.150 | −12.464 | 1.00 | 13.33 | A |
| ATOM | 3953 | C | SER | A | 504 | 65.860 | 67.487 | −15.570 | 1.00 | 12.34 | A |
| ATOM | 3954 | O | SER | A | 504 | 66.719 | 66.814 | −16.143 | 1.00 | 12.28 | A |
| ATOM | 3955 | N | VAL | A | 505 | 65.091 | 68.363 | −16.207 | 1.00 | 12.19 | A |
| ATOM | 3956 | CA | VAL | A | 505 | 65.237 | 68.577 | −17.644 | 1.00 | 12.76 | A |
| ATOM | 3957 | CB | VAL | A | 505 | 64.228 | 69.624 | −18.166 | 1.00 | 12.70 | A |
| ATOM | 3958 | CG1 | VAL | A | 505 | 64.573 | 70.019 | −19.607 | 1.00 | 12.02 | A |
| ATOM | 3959 | CG2 | VAL | A | 505 | 62.811 | 69.051 | −18.108 | 1.00 | 12.78 | A |
| ATOM | 3960 | C | VAL | A | 505 | 66.651 | 69.043 | −17.973 | 1.00 | 13.11 | A |
| ATOM | 3961 | O | VAL | A | 505 | 67.276 | 68.554 | −18.915 | 1.00 | 13.42 | A |
| ATOM | 3962 | N | TYR | A | 506 | 67.161 | 69.983 | −17.188 | 1.00 | 13.48 | A |
| ATOM | 3963 | CA | TYR | A | 506 | 68.503 | 70.496 | −17.423 | 1.00 | 14.99 | A |
| ATOM | 3964 | CB | TYR | A | 506 | 68.825 | 71.593 | −16.406 | 1.00 | 16.52 | A |
| ATOM | 3965 | CG | TYR | A | 506 | 70.166 | 72.239 | −16.635 | 1.00 | 18.96 | A |
| ATOM | 3966 | CD1 | TYR | A | 506 | 70.475 | 72.822 | −17.865 | 1.00 | 20.22 | A |
| ATOM | 3967 | CE1 | TYR | A | 506 | 71.722 | 73.393 | −18.098 | 1.00 | 22.60 | A |
| ATOM | 3968 | CD2 | TYR | A | 506 | 71.139 | 72.244 | −15.638 | 1.00 | 19.62 | A |
| ATOM | 3969 | CE2 | TYR | A | 506 | 72.394 | 72.811 | −15.864 | 1.00 | 22.32 | A |
| ATOM | 3970 | CZ | TYR | A | 506 | 72.674 | 73.380 | −17.097 | 1.00 | 22.77 | A |
| ATOM | 3971 | OH | TYR | A | 506 | 73.914 | 73.929 | −17.337 | 1.00 | 27.54 | A |
| ATOM | 3972 | C | TYR | A | 506 | 69.561 | 69.385 | −17.359 | 1.00 | 14.96 | A |
| ATOM | 3973 | O | TYR | A | 506 | 70.468 | 69.325 | −18.195 | 1.00 | 14.30 | A |
| ATOM | 3974 | N | ARG | A | 507 | 69.435 | 68.499 | −16.377 | 1.00 | 14.25 | A |
| ATOM | 3975 | CA | ARG | A | 507 | 70.384 | 67.399 | −16.214 | 1.00 | 14.23 | A |
| ATOM | 3976 | CB | ARG | A | 507 | 70.197 | 66.751 | −14.837 | 1.00 | 14.62 | A |
| ATOM | 3977 | CG | ARG | A | 507 | 71.238 | 65.694 | −14.492 | 1.00 | 15.49 | A |
| ATOM | 3978 | CD | ARG | A | 507 | 71.041 | 65.175 | −13.075 | 1.00 | 16.94 | A |
| ATOM | 3979 | NE | ARG | A | 507 | 72.055 | 64.190 | −12.707 | 1.00 | 18.67 | A |
| ATOM | 3980 | CZ | ARG | A | 507 | 72.170 | 63.649 | −11.497 | 1.00 | 18.55 | A |
| ATOM | 3981 | NH1 | ARG | A | 507 | 71.333 | 63.996 | −10.527 | 1.00 | 18.82 | A |
| ATOM | 3982 | NH2 | ARG | A | 507 | 73.122 | 62.758 | −11.256 | 1.00 | 19.58 | A |
| ATOM | 3983 | C | ARG | A | 507 | 70.247 | 66.334 | −17.310 | 1.00 | 13.93 | A |
| ATOM | 3984 | O | ARG | A | 507 | 71.241 | 65.776 | −17.776 | 1.00 | 12.93 | A |
| ATOM | 3985 | N | LEU | A | 508 | 69.016 | 66.062 | −17.723 | 1.00 | 13.04 | A |
| ATOM | 3986 | CA | LEU | A | 508 | 68.758 | 65.059 | −18.752 | 1.00 | 13.40 | A |
| ATOM | 3987 | CB | LEU | A | 508 | 67.274 | 64.669 | −18.738 | 1.00 | 12.96 | A |
| ATOM | 3988 | CG | LEU | A | 508 | 66.779 | 63.783 | −17.588 | 1.00 | 12.57 | A |
| ATOM | 3989 | CD1 | LEU | A | 508 | 65.251 | 63.836 | −17.521 | 1.00 | 12.21 | A |
| ATOM | 3990 | CD2 | LEU | A | 508 | 67.264 | 62.354 | −17.793 | 1.00 | 12.51 | A |
| ATOM | 3991 | C | LEU | A | 508 | 69.139 | 65.486 | −20.171 | 1.00 | 13.63 | A |
| ATOM | 3992 | O | LEU | A | 508 | 69.386 | 64.637 | −21.031 | 1.00 | 13.67 | A |
| ATOM | 3993 | N | LEU | A | 509 | 69.188 | 66.790 | −20.422 | 1.00 | 13.58 | A |
| ATOM | 3994 | CA | LEU | A | 509 | 69.494 | 67.278 | −21.763 | 1.00 | 13.53 | A |
| ATOM | 3995 | CB | LEU | A | 509 | 68.302 | 68.073 | −22.303 | 1.00 | 12.65 | A |
| ATOM | 3996 | CG | LEU | A | 509 | 67.030 | 67.266 | −22.577 | 1.00 | 12.79 | A |
| ATOM | 3997 | CD1 | LEU | A | 509 | 65.914 | 68.205 | −23.012 | 1.00 | 11.70 | A |
| ATOM | 3998 | CD2 | LEU | A | 509 | 67.301 | 66.222 | −23.657 | 1.00 | 12.41 | A |
| ATOM | 3999 | C | LEU | A | 509 | 70.766 | 68.107 | −21.916 | 1.00 | 14.29 | A |
| ATOM | 4000 | O | LEU | A | 509 | 70.915 | 68.837 | −22.900 | 1.00 | 14.16 | A |
| ATOM | 4001 | N | THR | A | 510 | 71.680 | 67.992 | −20.957 | 1.00 | 14.27 | A |
| ATOM | 4002 | CA | THR | A | 510 | 72.938 | 68.731 | −21.015 | 1.00 | 14.89 | A |
| ATOM | 4003 | CB | THR | A | 510 | 73.083 | 69.678 | −19.803 | 1.00 | 14.99 | A |
| ATOM | 4004 | OG1 | THR | A | 510 | 71.994 | 70.609 | −19.796 | 1.00 | 13.54 | A |
| ATOM | 4005 | CG2 | THR | A | 510 | 74.400 | 70.455 | −19.872 | 1.00 | 15.03 | A |
| ATOM | 4006 | C | THR | A | 510 | 74.100 | 67.744 | −21.022 | 1.00 | 15.57 | A |
| ATOM | 4007 | O | THR | A | 510 | 74.106 | 66.784 | −20.252 | 1.00 | 14.99 | A |
| ATOM | 4008 | N | LYS | A | 511 | 75.075 | 67.967 | −21.900 | 1.00 | 16.82 | A |
| ATOM | 4009 | CA | LYS | A | 511 | 76.234 | 67.075 | −21.972 | 1.00 | 18.12 | A |
| ATOM | 4010 | CB | LYS | A | 511 | 77.309 | 67.650 | −22.902 | 1.00 | 19.84 | A |
| ATOM | 4011 | CG | LYS | A | 511 | 78.515 | 66.740 | −23.058 | 1.00 | 22.03 | A |
| ATOM | 4012 | CD | LYS | A | 511 | 79.563 | 67.323 | −23.988 | 1.00 | 24.78 | A |
| ATOM | 4013 | CE | LYS | A | 511 | 80.759 | 66.385 | −24.094 | 1.00 | 26.41 | A |
| ATOM | 4014 | NZ | LYS | A | 511 | 81.834 | 66.924 | −24.969 | 1.00 | 28.84 | A |
| ATOM | 4015 | C | LYS | A | 511 | 76.789 | 66.911 | −20.564 | 1.00 | 17.91 | A |
| ATOM | 4016 | O | LYS | A | 511 | 77.076 | 67.895 | −19.879 | 1.00 | 17.65 | A |
| ATOM | 4017 | N | PRO | A | 512 | 76.943 | 65.658 | −20.110 | 1.00 | 18.35 | A |
| ATOM | 4018 | CD | PRO | A | 512 | 76.675 | 64.422 | −20.868 | 1.00 | 18.80 | A |
| ATOM | 4019 | CA | PRO | A | 512 | 77.455 | 65.343 | −18.773 | 1.00 | 18.48 | A |
| ATOM | 4020 | CB | PRO | A | 512 | 77.711 | 63.840 | −18.854 | 1.00 | 19.18 | A |
| ATOM | 4021 | CG | PRO | A | 512 | 76.629 | 63.379 | −19.773 | 1.00 | 19.70 | A |
| ATOM | 4022 | C | PRO | A | 512 | 78.685 | 66.118 | −18.299 | 1.00 | 18.38 | A |
| ATOM | 4023 | O | PRO | A | 512 | 78.698 | 66.633 | −17.182 | 1.00 | 18.96 | A |
| ATOM | 4024 | N | SER | A | 513 | 79.714 | 66.199 | −19.137 | 1.00 | 19.08 | A |
| ATOM | 4025 | CA | SER | A | 513 | 80.938 | 66.898 | −18.754 | 1.00 | 18.72 | A |
| ATOM | 4026 | CB | SER | A | 513 | 82.086 | 66.503 | −19.694 | 1.00 | 19.04 | A |
| ATOM | 4027 | OG | SER | A | 513 | 81.770 | 66.768 | −21.050 | 1.00 | 18.98 | A |
| ATOM | 4028 | C | SER | A | 513 | 80.800 | 68.421 | −18.701 | 1.00 | 18.92 | A |
| ATOM | 4029 | O | SER | A | 513 | 81.720 | 69.113 | −18.266 | 1.00 | 18.57 | A |
| ATOM | 4030 | N | ILE | A | 514 | 79.651 | 68.933 | −19.134 | 1.00 | 18.74 | A |
| ATOM | 4031 | CA | ILE | A | 514 | 79.383 | 70.371 | −19.131 | 1.00 | 19.04 | A |
| ATOM | 4032 | CB | ILE | A | 514 | 78.787 | 70.831 | −20.495 | 1.00 | 20.04 | A |
| ATOM | 4033 | CG2 | ILE | A | 514 | 78.309 | 72.281 | −20.407 | 1.00 | 21.10 | A |
| ATOM | 4034 | CG1 | ILE | A | 514 | 79.837 | 70.686 | −21.598 | 1.00 | 21.00 | A |
| ATOM | 4035 | CD1 | ILE | A | 514 | 79.333 | 71.051 | −22.983 | 1.00 | 22.47 | A |
| ATOM | 4036 | C | ILE | A | 514 | 78.395 | 70.729 | −18.016 | 1.00 | 18.79 | A |
| ATOM | 4037 | O | ILE | A | 514 | 78.419 | 71.838 | −17.482 | 1.00 | 17.84 | A |
| ATOM | 4038 | N | TYR | A | 515 | 77.531 | 69.778 | −17.671 | 1.00 | 18.53 | A |
| ATOM | 4039 | CA | TYR | A | 515 | 76.517 | 69.963 | −16.629 | 1.00 | 18.27 | A |
| ATOM | 4040 | CB | TYR | A | 515 | 75.828 | 68.619 | −16.371 | 1.00 | 18.00 | A |
| ATOM | 4041 | CG | TYR | A | 515 | 74.789 | 68.614 | −15.277 | 1.00 | 17.16 | A |
| ATOM | 4042 | CD1 | TYR | A | 515 | 73.637 | 69.398 | −15.369 | 1.00 | 17.45 | A |
| ATOM | 4043 | CE1 | TYR | A | 515 | 72.658 | 69.359 | −14.370 | 1.00 | 16.75 | A |
| ATOM | 4044 | CD2 | TYR | A | 515 | 74.940 | 67.793 | −14.160 | 1.00 | 17.50 | A |
| ATOM | 4045 | CE2 | TYR | A | 515 | 73.977 | 67.746 | −13.162 | 1.00 | 16.69 | A |
| ATOM | 4046 | CZ | TYR | A | 515 | 72.838 | 68.529 | −13.270 | 1.00 | 17.17 | A |
| ATOM | 4047 | OH | TYR | A | 515 | 71.886 | 68.473 | −12.276 | 1.00 | 17.04 | A |
| ATOM | 4048 | C | TYR | A | 515 | 77.102 | 70.529 | −15.330 | 1.00 | 18.11 | A |
| ATOM | 4049 | O | TYR | A | 515 | 77.924 | 69.890 | −14.674 | 1.00 | 18.22 | A |
| ATOM | 4050 | N | SER | A | 516 | 76.671 | 71.737 | −14.966 | 1.00 | 18.53 | A |
| ATOM | 4051 | CA | SER | A | 516 | 77.146 | 72.414 | −13.753 | 1.00 | 18.57 | A |
| ATOM | 4052 | CB | SER | A | 516 | 78.162 | 73.496 | −14.129 | 1.00 | 18.96 | A |
| ATOM | 4053 | OG | SER | A | 516 | 78.760 | 74.063 | −12.977 | 1.00 | 20.03 | A |
| ATOM | 4054 | C | SER | A | 516 | 75.936 | 73.047 | −13.066 | 1.00 | 19.08 | A |
| ATOM | 4055 | O | SER | A | 516 | 75.689 | 74.249 | −13.189 | 1.00 | 18.41 | A |
| ATOM | 4056 | N | PRO | A | 517 | 75.174 | 72.240 | −12.314 | 1.00 | 19.04 | A |
| ATOM | 4057 | CD | PRO | A | 517 | 75.364 | 70.793 | −12.109 | 1.00 | 19.14 | A |
| ATOM | 4058 | CA | PRO | A | 517 | 73.977 | 72.703 | −11.615 | 1.00 | 19.54 | A |
| ATOM | 4059 | CB | PRO | A | 517 | 73.238 | 71.402 | −11.327 | 1.00 | 19.43 | A |
| ATOM | 4060 | CG | PRO | A | 517 | 74.366 | 70.484 | −10.998 | 1.00 | 19.51 | A |
| ATOM | 4061 | C | PRO | A | 517 | 74.075 | 73.562 | −10.364 | 1.00 | 19.76 | A |
| ATOM | 4062 | O | PRO | A | 517 | 74.933 | 73.362 | −9.506 | 1.00 | 20.43 | A |
| ATOM | 4063 | N | ASP | A | 518 | 73.160 | 74.523 | −10.296 | 1.00 | 20.28 | A |
| ATOM | 4064 | CA | ASP | A | 518 | 72.983 | 75.411 | −9.157 | 1.00 | 20.46 | A |
| ATOM | 4065 | CB | ASP | A | 518 | 72.984 | 76.879 | −9.579 | 1.00 | 21.58 | A |
| ATOM | 4066 | CG | ASP | A | 518 | 72.662 | 77.813 | −8.424 | 1.00 | 22.93 | A |
| ATOM | 4067 | OD1 | ASP | A | 518 | 72.006 | 77.366 | −7.456 | 1.00 | 22.28 | A |
| ATOM | 4068 | OD2 | ASP | A | 518 | 73.048 | 78.999 | −8.487 | 1.00 | 24.17 | A |
| ATOM | 4069 | C | ASP | A | 518 | 71.562 | 74.998 | −8.784 | 1.00 | 20.16 | A |
| ATOM | 4070 | O | ASP | A | 518 | 70.602 | 75.422 | −9.426 | 1.00 | 19.99 | A |
| ATOM | 4071 | N | PHE | A | 519 | 71.435 | 74.159 | −7.764 | 1.00 | 20.05 | A |
| ATOM | 4072 | CA | PHE | A | 519 | 70.136 | 73.642 | −7.354 | 1.00 | 19.70 | A |
| ATOM | 4073 | CB | PHE | A | 519 | 70.331 | 72.607 | −6.247 | 1.00 | 19.92 | A |
| ATOM | 4074 | CG | PHE | A | 519 | 71.195 | 71.445 | −6.662 | 1.00 | 20.35 | A |
| ATOM | 4075 | CD1 | PHE | A | 519 | 70.936 | 70.760 | −7.848 | 1.00 | 20.35 | A |
| ATOM | 4076 | CD2 | PHE | A | 519 | 72.275 | 71.046 | −5.882 | 1.00 | 20.06 | A |
| ATOM | 4077 | CE1 | PHE | A | 519 | 71.741 | 69.694 | −8.253 | 1.00 | 20.49 | A |
| ATOM | 4078 | CE2 | PHE | A | 519 | 73.085 | 69.983 | −6.276 | 1.00 | 20.68 | A |
| ATOM | 4079 | CZ | PHE | A | 519 | 72.818 | 69.306 | −7.464 | 1.00 | 20.65 | A |
| ATOM | 4080 | C | PHE | A | 519 | 69.073 | 74.656 | −6.949 | 1.00 | 19.92 | A |
| ATOM | 4081 | O | PHE | A | 519 | 67.926 | 74.283 | −6.703 | 1.00 | 19.71 | A |
| ATOM | 4082 | N | SER | A | 520 | 69.438 | 75.932 | −6.901 | 1.00 | 20.32 | A |
| ATOM | 4083 | CA | SER | A | 520 | 68.482 | 76.974 | −6.534 | 1.00 | 20.88 | A |
| ATOM | 4084 | CB | SER | A | 520 | 69.072 | 77.879 | −5.452 | 1.00 | 21.33 | A |
| ATOM | 4085 | OG | SER | A | 520 | 70.122 | 78.674 | −5.981 | 1.00 | 22.27 | A |
| ATOM | 4086 | C | SER | A | 520 | 68.136 | 77.826 | −7.753 | 1.00 | 21.21 | A |
| ATOM | 4087 | O | SER | A | 520 | 67.272 | 78.700 | −7.687 | 1.00 | 20.94 | A |
| ATOM | 4088 | N | PHE | A | 521 | 68.816 | 77.558 | −8.862 | 1.00 | 20.71 | A |
| ATOM | 4089 | CA | PHE | A | 521 | 68.625 | 78.310 | −10.096 | 1.00 | 20.88 | A |
| ATOM | 4090 | CB | PHE | A | 521 | 69.932 | 78.310 | −10.897 | 1.00 | 22.15 | A |
| ATOM | 4091 | CG | PHE | A | 521 | 69.950 | 79.292 | −12.034 | 1.00 | 23.36 | A |
| ATOM | 4092 | CD1 | PHE | A | 521 | 70.078 | 80.657 | −11.787 | 1.00 | 24.36 | A |
| ATOM | 4093 | CD2 | PHE | A | 521 | 69.817 | 78.856 | −13.349 | 1.00 | 23.34 | A |
| ATOM | 4094 | CE1 | PHE | A | 521 | 70.071 | 81.578 | −12.837 | 1.00 | 24.48 | A |
| ATOM | 4095 | CE2 | PHE | A | 521 | 69.808 | 79.765 | −14.405 | 1.00 | 23.79 | A |
| ATOM | 4096 | CZ | PHE | A | 521 | 69.936 | 81.130 | −14.149 | 1.00 | 24.57 | A |
| ATOM | 4097 | C | PHE | A | 521 | 67.497 | 77.768 | −10.973 | 1.00 | 20.45 | A |
| ATOM | 4098 | O | PHE | A | 521 | 67.238 | 76.567 | −10.998 | 1.00 | 19.90 | A |
| ATOM | 4099 | N | SER | A | 522 | 66.828 | 78.666 | −11.693 | 1.00 | 20.31 | A |
| ATOM | 4100 | CA | SER | A | 522 | 65.746 | 78.274 | −12.588 | 1.00 | 19.82 | A |
| ATOM | 4101 | CB | SER | A | 522 | 64.569 | 79.250 | −12.474 | 1.00 | 20.87 | A |
| ATOM | 4102 | OG | SER | A | 522 | 63.902 | 79.102 | −11.230 | 1.00 | 24.21 | A |
| ATOM | 4103 | C | SER | A | 522 | 66.259 | 78.251 | −14.023 | 1.00 | 18.66 | A |
| ATOM | 4104 | O | SER | A | 522 | 66.316 | 79.286 | −14.686 | 1.00 | 18.49 | A |
| ATOM | 4105 | N | TYR | A | 523 | 66.643 | 77.070 | −14.495 | 1.00 | 17.51 | A |
| ATOM | 4106 | CA | TYR | A | 523 | 67.149 | 76.923 | −15.854 | 1.00 | 16.64 | A |
| ATOM | 4107 | CB | TYR | A | 523 | 67.888 | 75.592 | −16.013 | 1.00 | 17.45 | A |
| ATOM | 4108 | CG | TYR | A | 523 | 69.163 | 75.529 | −15.213 | 1.00 | 19.19 | A |
| ATOM | 4109 | CD1 | TYR | A | 523 | 69.191 | 74.941 | −13.949 | 1.00 | 18.97 | A |
| ATOM | 4110 | CE1 | TYR | A | 523 | 70.363 | 74.934 | −13.189 | 1.00 | 20.14 | A |
| ATOM | 4111 | CD2 | TYR | A | 523 | 70.338 | 76.106 | −15.701 | 1.00 | 19.40 | A |
| ATOM | 4112 | CE2 | TYR | A | 523 | 71.503 | 76.106 | −14.954 | 1.00 | 19.85 | A |
| ATOM | 4113 | CZ | TYR | A | 523 | 71.511 | 75.521 | −13.700 | 1.00 | 20.28 | A |
| ATOM | 4114 | OH | TYR | A | 523 | 72.669 | 75.540 | −12.955 | 1.00 | 20.88 | A |
| ATOM | 4115 | C | TYR | A | 523 | 66.023 | 77.002 | −16.865 | 1.00 | 16.16 | A |
| ATOM | 4116 | O | TYR | A | 523 | 66.225 | 77.440 | −18.000 | 1.00 | 15.44 | A |
| ATOM | 4117 | N | PHE | A | 524 | 64.838 | 76.560 | −16.452 | 1.00 | 15.23 | A |
| ATOM | 4118 | CA | PHE | A | 524 | 63.664 | 76.601 | −17.313 | 1.00 | 15.11 | A |
| ATOM | 4119 | CB | PHE | A | 524 | 63.294 | 75.216 | −17.852 | 1.00 | 14.27 | A |
| ATOM | 4120 | CG | PHE | A | 524 | 64.335 | 74.590 | −18.724 | 1.00 | 13.80 | A |
| ATOM | 4121 | CD1 | PHE | A | 524 | 65.403 | 73.897 | −18.166 | 1.00 | 14.22 | A |
| ATOM | 4122 | CD2 | PHE | A | 524 | 64.226 | 74.657 | −20.112 | 1.00 | 13.29 | A |
| ATOM | 4123 | CE1 | PHE | A | 524 | 66.349 | 73.274 | −18.977 | 1.00 | 14.06 | A |
| ATOM | 4124 | CE2 | PHE | A | 524 | 65.166 | 74.038 | −20.932 | 1.00 | 12.89 | A |
| ATOM | 4125 | CZ | PHE | A | 524 | 66.231 | 73.344 | −20.360 | 1.00 | 13.48 | A |
| ATOM | 4126 | C | PHE | A | 524 | 62.461 | 77.103 | −16.537 | 1.00 | 15.93 | A |
| ATOM | 4127 | O | PHE | A | 524 | 62.394 | 76.980 | −15.314 | 1.00 | 15.29 | A |
| ATOM | 4128 | N | THR | A | 525 | 61.510 | 77.661 | −17.274 | 1.00 | 16.53 | A |
| ATOM | 4129 | CA | THR | A | 525 | 60.265 | 78.145 | −16.708 | 1.00 | 18.21 | A |
| ATOM | 4130 | CB | THR | A | 525 | 60.026 | 79.627 | −17.050 | 1.00 | 18.69 | A |
| ATOM | 4131 | OG1 | THR | A | 525 | 61.031 | 80.431 | −16.419 | 1.00 | 22.90 | A |
| ATOM | 4132 | CG2 | THR | A | 525 | 58.659 | 80.072 | −16.565 | 1.00 | 21.52 | A |
| ATOM | 4133 | C | THR | A | 525 | 59.181 | 77.308 | −17.376 | 1.00 | 17.50 | A |
| ATOM | 4134 | O | THR | A | 525 | 59.190 | 77.147 | −18.596 | 1.00 | 16.90 | A |
| ATOM | 4135 | N | LEU | A | 526 | 58.265 | 76.757 | −16.587 | 1.00 | 16.82 | A |
| ATOM | 4136 | CA | LEU | A | 526 | 57.184 | 75.968 | −17.159 | 1.00 | 17.40 | A |
| ATOM | 4137 | CB | LEU | A | 526 | 56.457 | 75.166 | −16.072 | 1.00 | 18.26 | A |
| ATOM | 4138 | CG | LEU | A | 526 | 57.000 | 73.796 | −15.667 | 1.00 | 18.92 | A |
| ATOM | 4139 | CD1 | LEU | A | 526 | 56.213 | 73.271 | −14.472 | 1.00 | 19.91 | A |
| ATOM | 4140 | CD2 | LEU | A | 526 | 56.887 | 72.833 | −16.837 | 1.00 | 19.01 | A |
| ATOM | 4141 | C | LEU | A | 526 | 56.184 | 76.904 | −17.832 | 1.00 | 17.33 | A |
| ATOM | 4142 | O | LEU | A | 526 | 55.920 | 78.000 | −17.335 | 1.00 | 17.26 | A |
| ATOM | 4143 | N | ASP | A | 527 | 55.649 | 76.481 | −18.971 | 1.00 | 16.38 | A |
| ATOM | 4144 | CA | ASP | A | 527 | 54.651 | 77.271 | −19.674 | 1.00 | 17.04 | A |
| ATOM | 4145 | CB | ASP | A | 527 | 55.124 | 77.631 | −21.085 | 1.00 | 17.59 | A |
| ATOM | 4146 | CG | ASP | A | 527 | 54.133 | 78.520 | −21.819 | 1.00 | 18.89 | A |
| ATOM | 4147 | OD1 | ASP | A | 527 | 53.867 | 79.640 | −21.329 | 1.00 | 18.47 | A |
| ATOM | 4148 | OD2 | ASP | A | 527 | 53.620 | 78.097 | −22.879 | 1.00 | 18.66 | A |
| ATOM | 4149 | C | ASP | A | 527 | 53.409 | 76.396 | −19.751 | 1.00 | 16.37 | A |
| ATOM | 4150 | O | ASP | A | 527 | 53.434 | 75.340 | −20.375 | 1.00 | 17.71 | A |
| ATOM | 4151 | N | ASP | A | 528 | 52.337 | 76.825 | −19.094 | 1.00 | 16.05 | A |
| ATOM | 4152 | CA | ASP | A | 528 | 51.084 | 76.076 | −19.078 | 1.00 | 14.81 | A |
| ATOM | 4153 | CB | ASP | A | 528 | 50.703 | 75.718 | −17.638 | 1.00 | 14.73 | A |
| ATOM | 4154 | CG | ASP | A | 528 | 49.663 | 74.612 | −17.561 | 1.00 | 14.72 | A |
| ATOM | 4155 | OD1 | ASP | A | 528 | 48.648 | 74.677 | −18.288 | 1.00 | 13.92 | A |
| ATOM | 4156 | OD2 | ASP | A | 528 | 49.863 | 73.674 | −16.763 | 1.00 | 15.10 | A |
| ATOM | 4157 | C | ASP | A | 528 | 50.009 | 76.963 | −19.691 | 1.00 | 15.02 | A |
| ATOM | 4158 | O | ASP | A | 528 | 49.667 | 78.009 | −19.144 | 1.00 | 13.40 | A |
| ATOM | 4159 | N | SER | A | 529 | 49.471 | 76.546 | −20.829 | 1.00 | 15.55 | A |
| ATOM | 4160 | CA | SER | A | 529 | 48.462 | 77.355 | −21.494 | 1.00 | 17.05 | A |
| ATOM | 4161 | CB | SER | A | 529 | 48.464 | 77.064 | −22.998 | 1.00 | 18.24 | A |
| ATOM | 4162 | OG | SER | A | 529 | 47.861 | 75.815 | −23.262 | 1.00 | 23.59 | A |
| ATOM | 4163 | C | SER | A | 529 | 47.055 | 77.159 | −20.938 | 1.00 | 16.64 | A |
| ATOM | 4164 | O | SER | A | 529 | 46.149 | 77.913 | −21.283 | 1.00 | 16.93 | A |
| ATOM | 4165 | N | ARG | A | 530 | 46.866 | 76.169 | −20.070 | 1.00 | 15.67 | A |
| ATOM | 4166 | CA | ARG | A | 530 | 45.533 | 75.927 | −19.535 | 1.00 | 16.21 | A |
| ATOM | 4167 | CB | ARG | A | 530 | 45.033 | 74.562 | −20.015 | 1.00 | 15.39 | A |
| ATOM | 4168 | CG | ARG | A | 530 | 44.928 | 74.520 | −21.531 | 1.00 | 15.38 | A |
| ATOM | 4169 | CD | ARG | A | 530 | 44.291 | 73.247 | −22.059 | 1.00 | 15.32 | A |
| ATOM | 4170 | NE | ARG | A | 530 | 45.035 | 72.053 | −21.673 | 1.00 | 15.99 | A |
| ATOM | 4171 | CZ | ARG | A | 530 | 44.808 | 70.845 | −22.178 | 1.00 | 17.04 | A |
| ATOM | 4172 | NH1 | ARG | A | 530 | 43.858 | 70.681 | −23.091 | 1.00 | 16.99 | A |
| ATOM | 4173 | NH2 | ARG | A | 530 | 45.524 | 69.802 | −21.771 | 1.00 | 16.09 | A |
| ATOM | 4174 | C | ARG | A | 530 | 45.349 | 76.075 | −18.029 | 1.00 | 16.59 | A |
| ATOM | 4175 | O | ARG | A | 530 | 44.277 | 75.781 | −17.501 | 1.00 | 16.64 | A |
| ATOM | 4176 | N | TRP | A | 531 | 46.388 | 76.529 | −17.337 | 1.00 | 15.99 | A |
| ATOM | 4177 | CA | TRP | A | 531 | 46.277 | 76.760 | −15.900 | 1.00 | 17.02 | A |
| ATOM | 4178 | CB | TRP | A | 531 | 46.519 | 75.489 | −15.085 | 1.00 | 16.79 | A |
| ATOM | 4179 | CG | TRP | A | 531 | 46.420 | 75.776 | −13.618 | 1.00 | 18.61 | A |
| ATOM | 4180 | CD2 | TRP | A | 531 | 45.218 | 75.854 | −12.845 | 1.00 | 18.70 | A |
| ATOM | 4181 | CE2 | TRP | A | 531 | 45.583 | 76.258 | −11.540 | 1.00 | 19.61 | A |
| ATOM | 4182 | CE3 | TRP | A | 531 | 43.866 | 75.625 | −13.127 | 1.00 | 19.31 | A |
| ATOM | 4183 | CD1 | TRP | A | 531 | 47.440 | 76.124 | −12.773 | 1.00 | 18.69 | A |
| ATOM | 4184 | NE1 | TRP | A | 531 | 46.944 | 76.416 | −11.523 | 1.00 | 19.66 | A |
| ATOM | 4185 | CZ2 | TRP | A | 531 | 44.642 | 76.438 | −10.520 | 1.00 | 20.36 | A |
| ATOM | 4186 | CZ3 | TRP | A | 531 | 42.928 | 75.806 | −12.111 | 1.00 | 19.92 | A |
| ATOM | 4187 | CH2 | TRP | A | 531 | 43.323 | 76.207 | −10.825 | 1.00 | 20.15 | A |
| ATOM | 4188 | C | TRP | A | 531 | 47.247 | 77.833 | −15.433 | 1.00 | 17.11 | A |
| ATOM | 4189 | O | TRP | A | 531 | 48.445 | 77.745 | −15.691 | 1.00 | 16.78 | A |
| ATOM | 4190 | N | PRO | A | 532 | 46.740 | 78.855 | −14.724 | 1.00 | 18.18 | A |
| ATOM | 4191 | CD | PRO | A | 532 | 47.581 | 79.813 | −13.986 | 1.00 | 19.11 | A |
| ATOM | 4192 | CA | PRO | A | 532 | 45.327 | 79.028 | −14.362 | 1.00 | 19.02 | A |
| ATOM | 4193 | CB | PRO | A | 532 | 45.353 | 80.248 | −13.441 | 1.00 | 19.35 | A |
| ATOM | 4194 | CG | PRO | A | 532 | 46.705 | 80.142 | −12.795 | 1.00 | 19.62 | A |
| ATOM | 4195 | C | PRO | A | 532 | 44.421 | 79.230 | −15.572 | 1.00 | 20.08 | A |
| ATOM | 4196 | O | PRO | A | 532 | 43.202 | 79.077 | −15.479 | 1.00 | 19.73 | A |
| ATOM | 4197 | N | GLY | A | 533 | 45.024 | 79.574 | −16.705 | 1.00 | 20.53 | A |
| ATOM | 4198 | CA | GLY | A | 533 | 44.257 | 79.769 | −17.920 | 1.00 | 22.81 | A |
| ATOM | 4199 | C | GLY | A | 533 | 44.068 | 81.218 | −18.320 | 1.00 | 24.62 | A |
| ATOM | 4200 | O | GLY | A | 533 | 44.043 | 82.113 | −17.472 | 1.00 | 24.61 | A |
| ATOM | 4201 | N | SER | A | 534 | 43.938 | 81.447 | −19.624 | 1.00 | 26.45 | A |
| ATOM | 4202 | CA | SER | A | 534 | 43.739 | 82.791 | −20.153 | 1.00 | 27.99 | A |
| ATOM | 4203 | CB | SER | A | 534 | 43.665 | 82.746 | −21.682 | 1.00 | 29.27 | A |
| ATOM | 4204 | OG | SER | A | 534 | 43.340 | 84.017 | −22.215 | 1.00 | 31.07 | A |
| ATOM | 4205 | C | SER | A | 534 | 42.446 | 83.369 | −19.588 | 1.00 | 28.53 | A |
| ATOM | 4206 | O | SER | A | 534 | 41.402 | 82.714 | −19.617 | 1.00 | 28.85 | A |
| ATOM | 4207 | N | GLY | A | 535 | 42.518 | 84.592 | −19.072 | 1.00 | 29.09 | A |
| ATOM | 4208 | CA | GLY | A | 535 | 41.340 | 85.221 | −18.504 | 1.00 | 29.80 | A |
| ATOM | 4209 | C | GLY | A | 535 | 41.207 | 84.931 | −17.019 | 1.00 | 30.59 | A |
| ATOM | 4210 | O | GLY | A | 535 | 40.349 | 85.496 | −16.338 | 1.00 | 30.53 | A |
| ATOM | 4211 | N | VAL | A | 536 | 42.055 | 84.041 | −16.516 | 1.00 | 31.00 | A |
| ATOM | 4212 | CA | VAL | A | 536 | 42.038 | 83.680 | −15.104 | 1.00 | 31.98 | A |
| ATOM | 4213 | CB | VAL | A | 536 | 42.135 | 82.145 | −14.918 | 1.00 | 32.31 | A |
| ATOM | 4214 | CG1 | VAL | A | 536 | 42.095 | 81.791 | −13.439 | 1.00 | 31.89 | A |
| ATOM | 4215 | CG2 | VAL | A | 536 | 40.997 | 81.459 | −15.658 | 1.00 | 31.94 | A |
| ATOM | 4216 | C | VAL | A | 536 | 43.218 | 84.343 | −14.400 | 1.00 | 32.83 | A |
| ATOM | 4217 | O | VAL | A | 536 | 43.080 | 84.894 | −13.307 | 1.00 | 32.38 | A |
| ATOM | 4218 | N | GLU | A | 537 | 44.378 | 84.295 | −15.045 | 1.00 | 33.97 | A |
| ATOM | 4219 | CA | GLU | A | 537 | 45.592 | 84.881 | −14.495 | 1.00 | 35.29 | A |
| ATOM | 4220 | CB | GLU | A | 537 | 46.085 | 84.034 | −13.313 | 1.00 | 36.13 | A |
| ATOM | 4221 | CG | GLU | A | 537 | 47.534 | 84.273 | −12.900 | 1.00 | 37.68 | A |
| ATOM | 4222 | CD | GLU | A | 537 | 47.933 | 83.477 | −11.663 | 1.00 | 38.87 | A |
| ATOM | 4223 | OE1 | GLU | A | 537 | 49.149 | 83.273 | −11.450 | 1.00 | 39.08 | A |
| ATOM | 4224 | OE2 | GLU | A | 537 | 47.035 | 83.062 | −10.898 | 1.00 | 39.11 | A |
| ATOM | 4225 | C | GLU | A | 537 | 46.676 | 84.973 | −15.566 | 1.00 | 35.63 | A |
| ATOM | 4226 | O | GLU | A | 537 | 47.086 | 83.959 | −16.131 | 1.00 | 35.88 | A |
| ATOM | 4227 | N | ASP | A | 538 | 47.127 | 86.190 | −15.860 | 1.00 | 35.94 | A |
| ATOM | 4228 | CA | ASP | A | 538 | 48.183 | 86.371 | −16.853 | 1.00 | 35.97 | A |
| ATOM | 4229 | CB | ASP | A | 538 | 48.244 | 87.830 | −17.318 | 1.00 | 37.57 | A |
| ATOM | 4230 | CG | ASP | A | 538 | 49.235 | 88.037 | −18.452 | 1.00 | 39.01 | A |
| ATOM | 4231 | OD1 | ASP | A | 538 | 49.150 | 87.297 | −19.456 | 1.00 | 39.65 | A |
| ATOM | 4232 | OD2 | ASP | A | 538 | 50.094 | 88.938 | −18.344 | 1.00 | 40.23 | A |
| ATOM | 4233 | C | ASP | A | 538 | 49.477 | 85.981 | −16.154 | 1.00 | 35.09 | A |
| ATOM | 4234 | O | ASP | A | 538 | 50.132 | 86.812 | −15.524 | 1.00 | 35.01 | A |
| ATOM | 4235 | N | SER | A | 539 | 49.835 | 84.705 | −16.263 | 1.00 | 34.01 | A |
| ATOM | 4236 | CA | SER | A | 539 | 51.026 | 84.186 | −15.603 | 1.00 | 32.93 | A |
| ATOM | 4237 | CB | SER | A | 539 | 50.633 | 83.024 | −14.689 | 1.00 | 32.75 | A |
| ATOM | 4238 | OG | SER | A | 539 | 50.056 | 81.969 | −15.443 | 1.00 | 33.31 | A |
| ATOM | 4239 | C | SER | A | 539 | 52.145 | 83.722 | −16.525 | 1.00 | 31.91 | A |
| ATOM | 4240 | O | SER | A | 539 | 53.321 | 83.848 | −16.184 | 1.00 | 32.02 | A |
| ATOM | 4241 | N | ARG | A | 540 | 51.792 | 83.173 | −17.683 | 1.00 | 30.71 | A |
| ATOM | 4242 | CA | ARG | A | 540 | 52.816 | 82.685 | −18.596 | 1.00 | 29.23 | A |
| ATOM | 4243 | CB | ARG | A | 540 | 52.187 | 81.838 | −19.708 | 1.00 | 29.16 | A |
| ATOM | 4244 | CG | ARG | A | 540 | 50.939 | 82.406 | −20.335 | 1.00 | 27.91 | A |
| ATOM | 4245 | CD | ARG | A | 540 | 50.191 | 81.319 | −21.111 | 1.00 | 26.65 | A |
| ATOM | 4246 | NE | ARG | A | 540 | 51.057 | 80.605 | −22.049 | 1.00 | 23.55 | A |
| ATOM | 4247 | CZ | ARG | A | 540 | 50.631 | 80.056 | −23.182 | 1.00 | 23.62 | A |
| ATOM | 4248 | NH1 | ARG | A | 540 | 49.350 | 80.140 | −23.520 | 1.00 | 21.80 | A |
| ATOM | 4249 | NH2 | ARG | A | 540 | 51.482 | 79.431 | −23.983 | 1.00 | 22.12 | A |
| ATOM | 4250 | C | ARG | A | 540 | 53.686 | 83.795 | −19.170 | 1.00 | 28.47 | A |
| ATOM | 4251 | O | ARG | A | 540 | 53.212 | 84.887 | −19.490 | 1.00 | 28.48 | A |
| ATOM | 4252 | N | THR | A | 541 | 54.974 | 83.499 | −19.278 | 1.00 | 26.89 | A |
| ATOM | 4253 | CA | THR | A | 541 | 55.951 | 84.452 | −19.773 | 1.00 | 25.79 | A |
| ATOM | 4254 | CB | THR | A | 541 | 57.370 | 84.068 | −19.319 | 1.00 | 26.95 | A |
| ATOM | 4255 | OG1 | THR | A | 541 | 57.856 | 83.001 | −20.142 | 1.00 | 28.25 | A |
| ATOM | 4256 | CG2 | THR | A | 541 | 57.362 | 83.600 | −17.870 | 1.00 | 26.32 | A |
| ATOM | 4257 | C | THR | A | 541 | 55.969 | 84.547 | −21.288 | 1.00 | 24.23 | A |
| ATOM | 4258 | O | THR | A | 541 | 55.591 | 83.609 | −21.991 | 1.00 | 24.17 | A |
| ATOM | 4259 | N | THR | A | 542 | 56.411 | 85.697 | −21.784 | 1.00 | 22.20 | A |
| ATOM | 4260 | CA | THR | A | 542 | 56.523 | 85.917 | −23.214 | 1.00 | 19.97 | A |
| ATOM | 4261 | CB | THR | A | 542 | 56.167 | 87.371 | −23.609 | 1.00 | 20.43 | A |
| ATOM | 4262 | OG1 | THR | A | 542 | 54.789 | 87.630 | −23.320 | 1.00 | 19.24 | A |
| ATOM | 4263 | CG2 | THR | A | 542 | 56.419 | 87.594 | −25.098 | 1.00 | 19.35 | A |
| ATOM | 4264 | C | THR | A | 542 | 57.978 | 85.679 | −23.581 | 1.00 | 19.20 | A |
| ATOM | 4265 | O | THR | A | 542 | 58.884 | 86.149 | −22.889 | 1.00 | 18.80 | A |
| ATOM | 4266 | N | ILE | A | 543 | 58.207 | 84.921 | −24.644 | 1.00 | 17.47 | A |
| ATOM | 4267 | CA | ILE | A | 543 | 59.567 | 84.688 | −25.097 | 1.00 | 17.03 | A |
| ATOM | 4268 | CB | ILE | A | 543 | 59.649 | 83.461 | −26.023 | 1.00 | 16.71 | A |
| ATOM | 4269 | CG2 | ILE | A | 543 | 61.033 | 83.373 | −26.666 | 1.00 | 16.10 | A |
| ATOM | 4270 | CG1 | ILE | A | 543 | 59.335 | 82.194 | −25.215 | 1.00 | 16.14 | A |
| ATOM | 4271 | CD1 | ILE | A | 543 | 59.378 | 80.913 | −26.021 | 1.00 | 15.87 | A |
| ATOM | 4272 | C | ILE | A | 543 | 59.899 | 85.962 | −25.869 | 1.00 | 17.36 | A |
| ATOM | 4273 | O | ILE | A | 543 | 59.317 | 86.225 | −26.923 | 1.00 | 16.63 | A |
| ATOM | 4274 | N | ILE | A | 544 | 60.808 | 86.763 | −25.319 | 1.00 | 17.34 | A |
| ATOM | 4275 | CA | ILE | A | 544 | 61.189 | 88.025 | −25.935 | 1.00 | 17.68 | A |
| ATOM | 4276 | CB | ILE | A | 544 | 61.452 | 89.098 | −24.854 | 1.00 | 18.33 | A |
| ATOM | 4277 | CG2 | ILE | A | 544 | 61.827 | 90.424 | −25.504 | 1.00 | 18.51 | A |
| ATOM | 4278 | CG1 | ILE | A | 544 | 60.192 | 89.275 | −24.000 | 1.00 | 19.26 | A |
| ATOM | 4279 | CD1 | ILE | A | 544 | 60.307 | 90.343 | −22.934 | 1.00 | 21.06 | A |
| ATOM | 4280 | C | ILE | A | 544 | 62.412 | 87.877 | −26.832 | 1.00 | 17.90 | A |
| ATOM | 4281 | O | ILE | A | 544 | 63.518 | 87.601 | −26.367 | 1.00 | 17.14 | A |
| ATOM | 4282 | N | LEU | A | 545 | 62.191 | 88.053 | −28.129 | 1.00 | 17.81 | A |
| ATOM | 4283 | CA | LEU | A | 545 | 63.253 | 87.941 | −29.118 | 1.00 | 18.43 | A |
| ATOM | 4284 | CB | LEU | A | 545 | 62.913 | 86.836 | −30.120 | 1.00 | 17.98 | A |
| ATOM | 4285 | CG | LEU | A | 545 | 62.603 | 85.454 | −29.530 | 1.00 | 17.86 | A |
| ATOM | 4286 | CD1 | LEU | A | 545 | 62.153 | 84.516 | −30.640 | 1.00 | 17.14 | A |
| ATOM | 4287 | CD2 | LEU | A | 545 | 63.837 | 84.901 | −28.819 | 1.00 | 17.14 | A |
| ATOM | 4288 | C | LEU | A | 545 | 63.412 | 89.274 | −29.847 | 1.00 | 19.23 | A |
| ATOM | 4289 | O | LEU | A | 545 | 62.471 | 90.059 | −29.926 | 1.00 | 18.84 | A |
| ATOM | 4290 | N | GLY | A | 546 | 64.607 | 89.526 | −30.372 | 1.00 | 19.77 | A |
| ATOM | 4291 | CA | GLY | A | 546 | 64.851 | 90.766 | −31.087 | 1.00 | 20.96 | A |
| ATOM | 4292 | C | GLY | A | 546 | 66.276 | 90.848 | −31.598 | 1.00 | 21.61 | A |
| ATOM | 4293 | O | GLY | A | 546 | 67.199 | 90.368 | −30.949 | 1.00 | 20.57 | A |
| ATOM | 4294 | N | GLU | A | 547 | 66.456 | 91.463 | −32.761 | 1.00 | 23.26 | A |
| ATOM | 4295 | CA | GLU | A | 547 | 67.780 | 91.599 | −33.361 | 1.00 | 25.04 | A |
| ATOM | 4296 | CB | GLU | A | 547 | 67.702 | 92.459 | −34.624 | 1.00 | 27.57 | A |
| ATOM | 4297 | CG | GLU | A | 547 | 66.673 | 92.001 | −35.642 | 1.00 | 31.94 | A |
| ATOM | 4298 | CD | GLU | A | 547 | 66.538 | 92.977 | −36.800 | 1.00 | 34.18 | A |
| ATOM | 4299 | OE1 | GLU | A | 547 | 67.521 | 93.141 | −37.558 | 1.00 | 35.72 | A |
| ATOM | 4300 | OE2 | GLU | A | 547 | 65.452 | 93.584 | −36.946 | 1.00 | 35.67 | A |
| ATOM | 4301 | C | GLU | A | 547 | 68.771 | 92.241 | −32.397 | 1.00 | 24.63 | A |
| ATOM | 4302 | O | GLU | A | 547 | 69.950 | 91.886 | −32.372 | 1.00 | 24.67 | A |
| ATOM | 4303 | N | ASP | A | 548 | 68.286 | 93.190 | −31.605 | 1.00 | 23.96 | A |
| ATOM | 4304 | CA | ASP | A | 548 | 69.138 | 93.898 | −30.660 | 1.00 | 23.90 | A |
| ATOM | 4305 | CB | ASP | A | 548 | 68.716 | 95.368 | −30.582 | 1.00 | 24.37 | A |
| ATOM | 4306 | CG | ASP | A | 548 | 68.902 | 96.099 | −31.894 | 1.00 | 25.10 | A |
| ATOM | 4307 | OD1 | ASP | A | 548 | 70.041 | 96.129 | −32.403 | 1.00 | 25.66 | A |
| ATOM | 4308 | OD2 | ASP | A | 548 | 67.910 | 96.646 | −32.417 | 1.00 | 26.21 | A |
| ATOM | 4309 | C | ASP | A | 548 | 69.166 | 93.316 | −29.250 | 1.00 | 23.40 | A |
| ATOM | 4310 | O | ASP | A | 548 | 69.753 | 93.920 | −28.349 | 1.00 | 23.41 | A |
| ATOM | 4311 | N | ILE | A | 549 | 68.556 | 92.152 | −29.046 | 1.00 | 22.05 | A |
| ATOM | 4312 | CA | ILE | A | 549 | 68.546 | 91.568 | −27.707 | 1.00 | 21.36 | A |
| ATOM | 4313 | CB | ILE | A | 549 | 67.220 | 91.910 | −26.966 | 1.00 | 21.60 | A |
| ATOM | 4314 | CG2 | ILE | A | 549 | 66.031 | 91.355 | −27.727 | 1.00 | 21.55 | A |
| ATOM | 4315 | CG1 | ILE | A | 549 | 67.244 | 91.341 | −25.545 | 1.00 | 23.19 | A |
| ATOM | 4316 | CD1 | ILE | A | 549 | 68.321 | 91.933 | −24.664 | 1.00 | 24.78 | A |
| ATOM | 4317 | C | ILE | A | 549 | 68.766 | 90.057 | −27.654 | 1.00 | 20.15 | A |
| ATOM | 4318 | O | ILE | A | 549 | 69.594 | 89.577 | −26.883 | 1.00 | 19.89 | A |
| ATOM | 4319 | N | LEU | A | 550 | 68.038 | 89.311 | −28.477 | 1.00 | 19.54 | A |
| ATOM | 4320 | CA | LEU | A | 550 | 68.156 | 87.854 | −28.483 | 1.00 | 19.00 | A |
| ATOM | 4321 | CB | LEU | A | 550 | 67.484 | 87.281 | −27.231 | 1.00 | 19.06 | A |
| ATOM | 4322 | CG | LEU | A | 550 | 67.584 | 85.770 | −27.011 | 1.00 | 18.75 | A |
| ATOM | 4323 | CD1 | LEU | A | 550 | 69.034 | 85.393 | −26.760 | 1.00 | 18.02 | A |
| ATOM | 4324 | CD2 | LEU | A | 550 | 66.722 | 85.367 | −25.827 | 1.00 | 17.92 | A |
| ATOM | 4325 | C | LEU | A | 550 | 67.488 | 87.284 | −29.729 | 1.00 | 18.70 | A |
| ATOM | 4326 | O | LEU | A | 550 | 66.286 | 87.448 | −29.930 | 1.00 | 19.34 | A |
| ATOM | 4327 | N | PRO | A | 551 | 68.260 | 86.597 | −30.582 | 1.00 | 18.21 | A |
| ATOM | 4328 | CD | PRO | A | 551 | 69.734 | 86.532 | −30.610 | 1.00 | 18.48 | A |
| ATOM | 4329 | CA | PRO | A | 551 | 67.687 | 86.029 | −31.804 | 1.00 | 17.94 | A |
| ATOM | 4330 | CB | PRO | A | 551 | 68.907 | 85.875 | −32.709 | 1.00 | 18.38 | A |
| ATOM | 4331 | CG | PRO | A | 551 | 69.995 | 85.562 | −31.741 | 1.00 | 19.26 | A |
| ATOM | 4332 | C | PRO | A | 551 | 66.886 | 84.730 | −31.679 | 1.00 | 17.64 | A |
| ATOM | 4333 | O | PRO | A | 551 | 65.933 | 84.522 | −32.431 | 1.00 | 18.20 | A |
| ATOM | 4334 | N | SER | A | 552 | 67.253 | 83.861 | −30.743 | 1.00 | 16.28 | A |
| ATOM | 4335 | CA | SER | A | 552 | 66.539 | 82.593 | −30.604 | 1.00 | 15.64 | A |
| ATOM | 4336 | CB | SER | A | 552 | 67.260 | 81.497 | −31.393 | 1.00 | 15.46 | A |
| ATOM | 4337 | OG | SER | A | 552 | 68.562 | 81.286 | −30.881 | 1.00 | 16.20 | A |
| ATOM | 4338 | C | SER | A | 552 | 66.361 | 82.135 | −29.164 | 1.00 | 14.77 | A |
| ATOM | 4339 | O | SER | A | 552 | 66.983 | 82.665 | −28.246 | 1.00 | 14.68 | A |
| ATOM | 4340 | N | LYS | A | 553 | 65.509 | 81.132 | −28.988 | 1.00 | 14.88 | A |
| ATOM | 4341 | CA | LYS | A | 553 | 65.204 | 80.590 | −27.672 | 1.00 | 14.72 | A |
| ATOM | 4342 | CB | LYS | A | 553 | 63.999 | 81.333 | −27.082 | 1.00 | 15.55 | A |
| ATOM | 4343 | CG | LYS | A | 553 | 63.458 | 80.742 | −25.780 | 1.00 | 16.12 | A |
| ATOM | 4344 | CD | LYS | A | 553 | 64.486 | 80.803 | −24.659 | 1.00 | 16.06 | A |
| ATOM | 4345 | CE | LYS | A | 553 | 64.815 | 82.244 | −24.273 | 1.00 | 17.14 | A |
| ATOM | 4346 | NZ | LYS | A | 553 | 65.918 | 82.300 | −23.265 | 1.00 | 15.93 | A |
| ATOM | 4347 | C | LYS | A | 553 | 64.898 | 79.092 | −27.734 | 1.00 | 15.05 | A |
| ATOM | 4348 | O | LYS | A | 553 | 64.156 | 78.631 | −28.602 | 1.00 | 13.66 | A |
| ATOM | 4349 | N | HIS | A | 554 | 65.473 | 78.336 | −26.806 | 1.00 | 15.49 | A |
| ATOM | 4350 | CA | HIS | A | 554 | 65.231 | 76.900 | −26.747 | 1.00 | 15.75 | A |
| ATOM | 4351 | CB | HIS | A | 554 | 66.446 | 76.155 | −26.189 | 1.00 | 17.73 | A |
| ATOM | 4352 | CG | HIS | A | 554 | 67.584 | 76.035 | −27.152 | 1.00 | 19.69 | A |
| ATOM | 4353 | CD2 | HIS | A | 554 | 68.188 | 76.955 | −27.939 | 1.00 | 20.84 | A |
| ATOM | 4354 | ND1 | HIS | A | 554 | 68.259 | 74.851 | −27.359 | 1.00 | 21.35 | A |
| ATOM | 4355 | CE1 | HIS | A | 554 | 69.231 | 75.047 | −28.230 | 1.00 | 20.38 | A |
| ATOM | 4356 | NE2 | HIS | A | 554 | 69.211 | 76.316 | −28.597 | 1.00 | 21.79 | A |
| ATOM | 4357 | C | HIS | A | 554 | 64.040 | 76.598 | −25.851 | 1.00 | 15.45 | A |
| ATOM | 4358 | O | HIS | A | 554 | 63.872 | 77.221 | −24.802 | 1.00 | 15.13 | A |
| ATOM | 4359 | N | VAL | A | 555 | 63.223 | 75.641 | −26.280 | 1.00 | 14.33 | A |
| ATOM | 4360 | CA | VAL | A | 555 | 62.062 | 75.193 | −25.523 | 1.00 | 14.09 | A |
| ATOM | 4361 | CB | VAL | A | 555 | 60.725 | 75.652 | −26.153 | 1.00 | 13.60 | A |
| ATOM | 4362 | CG1 | VAL | A | 555 | 60.610 | 77.170 | −26.086 | 1.00 | 15.20 | A |
| ATOM | 4363 | CG2 | VAL | A | 555 | 60.616 | 75.158 | −27.584 | 1.00 | 13.42 | A |
| ATOM | 4364 | C | VAL | A | 555 | 62.110 | 73.670 | −25.528 | 1.00 | 13.62 | A |
| ATOM | 4365 | O | VAL | A | 555 | 62.595 | 73.061 | −26.487 | 1.00 | 14.40 | A |
| ATOM | 4366 | N | VAL | A | 556 | 61.606 | 73.058 | −24.463 | 1.00 | 12.29 | A |
| ATOM | 4367 | CA | VAL | A | 556 | 61.618 | 71.606 | −24.346 | 1.00 | 11.85 | A |
| ATOM | 4368 | CB | VAL | A | 556 | 62.659 | 71.151 | −23.288 | 1.00 | 11.87 | A |
| ATOM | 4369 | CG1 | VAL | A | 556 | 62.602 | 69.634 | −23.110 | 1.00 | 10.24 | A |
| ATOM | 4370 | CG2 | VAL | A | 556 | 64.056 | 71.590 | −23.704 | 1.00 | 11.21 | A |
| ATOM | 4371 | C | VAL | A | 556 | 60.259 | 71.052 | −23.929 | 1.00 | 11.36 | A |
| ATOM | 4372 | O | VAL | A | 556 | 59.624 | 71.583 | −23.022 | 1.00 | 10.98 | A |
| ATOM | 4373 | N | MET | A | 557 | 59.819 | 69.985 | −24.590 | 1.00 | 11.49 | A |
| ATOM | 4374 | CA | MET | A | 557 | 58.553 | 69.343 | −24.241 | 1.00 | 11.87 | A |
| ATOM | 4375 | CB | MET | A | 557 | 57.681 | 69.128 | −25.486 | 1.00 | 11.62 | A |
| ATOM | 4376 | CG | MET | A | 557 | 56.648 | 70.219 | −25.752 | 1.00 | 12.29 | A |
| ATOM | 4377 | SD | MET | A | 557 | 57.365 | 71.858 | −25.911 | 1.00 | 12.86 | A |
| ATOM | 4378 | CE | MET | A | 557 | 58.354 | 71.664 | −27.421 | 1.00 | 12.75 | A |
| ATOM | 4379 | C | MET | A | 557 | 58.805 | 67.992 | −23.572 | 1.00 | 11.62 | A |
| ATOM | 4380 | O | MET | A | 557 | 59.714 | 67.255 | −23.958 | 1.00 | 11.99 | A |
| ATOM | 4381 | N | HIS | A | 558 | 58.002 | 67.675 | −22.562 | 1.00 | 11.19 | A |
| ATOM | 4382 | CA | HIS | A | 558 | 58.114 | 66.397 | −21.864 | 1.00 | 10.78 | A |
| ATOM | 4383 | CB | HIS | A | 558 | 58.316 | 66.610 | −20.363 | 1.00 | 10.37 | A |
| ATOM | 4384 | CG | HIS | A | 558 | 58.227 | 65.348 | −19.558 | 1.00 | 11.12 | A |
| ATOM | 4385 | CD2 | HIS | A | 558 | 58.984 | 64.226 | −19.570 | 1.00 | 10.73 | A |
| ATOM | 4386 | ND1 | HIS | A | 558 | 57.259 | 65.146 | −18.596 | 1.00 | 10.46 | A |
| ATOM | 4387 | CE1 | HIS | A | 558 | 57.426 | 63.955 | −18.051 | 1.00 | 10.44 | A |
| ATOM | 4388 | NE2 | HIS | A | 558 | 58.466 | 63.377 | −18.623 | 1.00 | 11.40 | A |
| ATOM | 4389 | C | HIS | A | 558 | 56.828 | 65.611 | −22.086 | 1.00 | 10.70 | A |
| ATOM | 4390 | O | HIS | A | 558 | 55.738 | 66.191 | −22.090 | 1.00 | 11.40 | A |
| ATOM | 4391 | N | ASN | A | 559 | 56.962 | 64.299 | −22.280 | 1.00 | 9.85 | A |
| ATOM | 4392 | CA | ASN | A | 559 | 55.820 | 63.413 | −22.494 | 1.00 | 10.04 | A |
| ATOM | 4393 | CB | ASN | A | 559 | 55.880 | 62.807 | −23.902 | 1.00 | 10.29 | A |
| ATOM | 4394 | CG | ASN | A | 559 | 54.865 | 61.692 | −24.102 | 1.00 | 10.99 | A |
| ATOM | 4395 | OD1 | ASN | A | 559 | 53.762 | 61.738 | −23.557 | 1.00 | 10.85 | A |
| ATOM | 4396 | ND2 | ASN | A | 559 | 55.229 | 60.692 | −24.901 | 1.00 | 11.25 | A |
| ATOM | 4397 | C | ASN | A | 559 | 55.835 | 62.307 | −21.439 | 1.00 | 9.59 | A |
| ATOM | 4398 | O | ASN | A | 559 | 56.575 | 61.332 | −21.555 | 1.00 | 9.79 | A |
| ATOM | 4399 | N | THR | A | 560 | 55.012 | 62.455 | −20.409 | 1.00 | 9.86 | A |
| ATOM | 4400 | CA | THR | A | 560 | 54.989 | 61.472 | −19.331 | 1.00 | 10.97 | A |
| ATOM | 4401 | CB | THR | A | 560 | 54.244 | 62.042 | −18.102 | 1.00 | 11.06 | A |
| ATOM | 4402 | OG1 | THR | A | 560 | 54.560 | 61.255 | −16.949 | 1.00 | 10.92 | A |
| ATOM | 4403 | CG2 | THR | A | 560 | 52.737 | 62.039 | −18.336 | 1.00 | 10.12 | A |
| ATOM | 4404 | C | THR | A | 560 | 54.391 | 60.111 | −19.717 | 1.00 | 11.41 | A |
| ATOM | 4405 | O | THR | A | 560 | 54.603 | 59.112 | −19.017 | 1.00 | 11.04 | A |
| ATOM | 4406 | N | LEU | A | 561 | 53.667 | 60.069 | −20.834 | 1.00 | 10.95 | A |
| ATOM | 4407 | CA | LEU | A | 561 | 53.043 | 58.828 | −21.306 | 1.00 | 11.48 | A |
| ATOM | 4408 | CB | LEU | A | 561 | 51.918 | 59.153 | −22.295 | 1.00 | 12.27 | A |
| ATOM | 4409 | CG | LEU | A | 561 | 50.793 | 60.046 | −21.747 | 1.00 | 12.97 | A |
| ATOM | 4410 | CD1 | LEU | A | 561 | 49.728 | 60.265 | −22.818 | 1.00 | 12.43 | A |
| ATOM | 4411 | CD2 | LEU | A | 561 | 50.176 | 59.389 | −20.511 | 1.00 | 13.32 | A |
| ATOM | 4412 | C | LEU | A | 561 | 54.070 | 57.892 | −21.957 | 1.00 | 11.68 | A |
| ATOM | 4413 | O | LEU | A | 561 | 54.992 | 58.345 | −22.641 | 1.00 | 11.40 | A |
| ATOM | 4414 | N | PRO | A | 562 | 53.912 | 56.569 | −21.759 | 1.00 | 11.32 | A |
| ATOM | 4415 | CD | PRO | A | 562 | 52.919 | 55.940 | −20.867 | 1.00 | 10.81 | A |
| ATOM | 4416 | CA | PRO | A | 562 | 54.824 | 55.562 | −22.312 | 1.00 | 11.80 | A |
| ATOM | 4417 | CB | PRO | A | 562 | 54.605 | 54.367 | −21.389 | 1.00 | 10.79 | A |
| ATOM | 4418 | CG | PRO | A | 562 | 53.137 | 54.455 | −21.113 | 1.00 | 10.79 | A |
| ATOM | 4419 | C | PRO | A | 562 | 54.689 | 55.193 | −23.788 | 1.00 | 12.65 | A |
| ATOM | 4420 | O | PRO | A | 562 | 54.855 | 54.029 | −24.158 | 1.00 | 13.27 | A |
| ATOM | 4421 | N | HIS | A | 563 | 54.382 | 56.176 | −24.628 | 1.00 | 12.72 | A |
| ATOM | 4422 | CA | HIS | A | 563 | 54.294 | 55.951 | −26.066 | 1.00 | 13.06 | A |
| ATOM | 4423 | CB | HIS | A | 563 | 52.900 | 55.442 | −26.495 | 1.00 | 12.83 | A |
| ATOM | 4424 | CG | HIS | A | 563 | 51.757 | 56.313 | −26.069 | 1.00 | 13.57 | A |
| ATOM | 4425 | CD2 | HIS | A | 563 | 51.235 | 57.434 | −26.622 | 1.00 | 13.78 | A |
| ATOM | 4426 | ND1 | HIS | A | 563 | 50.971 | 56.027 | −24.972 | 1.00 | 13.58 | A |
| ATOM | 4427 | CE1 | HIS | A | 563 | 50.013 | 56.930 | −24.871 | 1.00 | 13.87 | A |
| ATOM | 4428 | NE2 | HIS | A | 563 | 50.151 | 57.796 | −25.860 | 1.00 | 14.57 | A |
| ATOM | 4429 | C | HIS | A | 563 | 54.628 | 57.248 | −26.786 | 1.00 | 13.25 | A |
| ATOM | 4430 | O | HIS | A | 563 | 54.463 | 58.332 | −26.225 | 1.00 | 13.58 | A |
| ATOM | 4431 | N | TRP | A | 564 | 55.133 | 57.142 | −28.009 | 1.00 | 13.21 | A |
| ATOM | 4432 | CA | TRP | A | 564 | 55.458 | 58.336 | −28.772 | 1.00 | 13.90 | A |
| ATOM | 4433 | CB | TRP | A | 564 | 55.932 | 57.978 | −30.181 | 1.00 | 14.45 | A |
| ATOM | 4434 | CG | TRP | A | 564 | 57.363 | 57.568 | −30.235 | 1.00 | 14.66 | A |
| ATOM | 4435 | CD2 | TRP | A | 564 | 58.487 | 58.433 | −30.411 | 1.00 | 15.15 | A |
| ATOM | 4436 | CE2 | TRP | A | 564 | 59.646 | 57.630 | −30.351 | 1.00 | 15.51 | A |
| ATOM | 4437 | CE3 | TRP | A | 564 | 58.628 | 59.814 | −30.613 | 1.00 | 15.70 | A |
| ATOM | 4438 | CD1 | TRP | A | 564 | 57.864 | 56.310 | −30.081 | 1.00 | 15.38 | A |
| ATOM | 4439 | NE1 | TRP | A | 564 | 59.238 | 56.337 | −30.148 | 1.00 | 15.68 | A |
| ATOM | 4440 | CZ2 | TRP | A | 564 | 60.934 | 58.159 | −30.484 | 1.00 | 15.36 | A |
| ATOM | 4441 | CZ3 | TRP | A | 564 | 59.911 | 60.343 | −30.747 | 1.00 | 15.81 | A |
| ATOM | 4442 | CH2 | TRP | A | 564 | 61.046 | 59.515 | −30.682 | 1.00 | 16.52 | A |
| ATOM | 4443 | C | TRP | A | 564 | 54.190 | 59.160 | −28.866 | 1.00 | 13.86 | A |
| ATOM | 4444 | O | TRP | A | 564 | 53.104 | 58.614 | −29.046 | 1.00 | 13.89 | A |
| ATOM | 4445 | N | ARG | A | 565 | 54.317 | 60.472 | −28.738 | 1.00 | 14.17 | A |
| ATOM | 4446 | CA | ARG | A | 565 | 53.135 | 61.311 | −28.821 | 1.00 | 14.31 | A |
| ATOM | 4447 | CB | ARG | A | 565 | 52.560 | 61.579 | −27.419 | 1.00 | 14.39 | A |
| ATOM | 4448 | CG | ARG | A | 565 | 51.352 | 62.521 | −27.424 | 1.00 | 15.52 | A |
| ATOM | 4449 | CD | ARG | A | 565 | 50.536 | 62.477 | −26.128 | 1.00 | 15.96 | A |
| ATOM | 4450 | NE | ARG | A | 565 | 51.334 | 62.761 | −24.940 | 1.00 | 15.97 | A |
| ATOM | 4451 | CZ | ARG | A | 565 | 50.831 | 63.221 | −23.796 | 1.00 | 15.92 | A |
| ATOM | 4452 | NH1 | ARG | A | 565 | 49.525 | 63.456 | −23.685 | 1.00 | 15.90 | A |
| ATOM | 4453 | NH2 | ARG | A | 565 | 51.631 | 63.440 | −22.762 | 1.00 | 14.90 | A |
| ATOM | 4454 | C | ARG | A | 565 | 53.385 | 62.627 | −29.525 | 1.00 | 14.40 | A |
| ATOM | 4455 | O | ARG | A | 565 | 54.436 | 63.251 | −29.361 | 1.00 | 13.99 | A |
| ATOM | 4456 | N | GLU | A | 566 | 52.413 | 63.020 | −30.339 | 1.00 | 14.96 | A |
| ATOM | 4457 | CA | GLU | A | 566 | 52.463 | 64.287 | −31.044 | 1.00 | 15.90 | A |
| ATOM | 4458 | CB | GLU | A | 566 | 52.236 | 64.109 | −32.546 | 1.00 | 17.55 | A |
| ATOM | 4459 | CG | GLU | A | 566 | 53.338 | 63.375 | −33.277 | 1.00 | 18.99 | A |
| ATOM | 4460 | CD | GLU | A | 566 | 53.140 | 63.416 | −34.783 | 1.00 | 20.96 | A |
| ATOM | 4461 | OE1 | GLU | A | 566 | 52.003 | 63.171 | −35.238 | 1.00 | 21.39 | A |
| ATOM | 4462 | OE2 | GLU | A | 566 | 54.119 | 63.688 | −35.507 | 1.00 | 21.79 | A |
| ATOM | 4463 | C | GLU | A | 566 | 51.325 | 65.102 | −30.456 | 1.00 | 16.15 | A |
| ATOM | 4464 | O | GLU | A | 566 | 50.262 | 64.561 | −30.137 | 1.00 | 16.46 | A |
| ATOM | 4465 | N | GLN | A | 567 | 51.555 | 66.397 | −30.294 | 1.00 | 15.49 | A |
| ATOM | 4466 | CA | GLN | A | 567 | 50.547 | 67.288 | −29.744 | 1.00 | 15.72 | A |
| ATOM | 4467 | CB | GLN | A | 567 | 50.471 | 67.152 | −28.216 | 1.00 | 17.10 | A |
| ATOM | 4468 | CG | GLN | A | 567 | 49.471 | 68.113 | −27.571 | 1.00 | 17.38 | A |
| ATOM | 4469 | CD | GLN | A | 567 | 49.793 | 68.433 | −26.119 | 1.00 | 18.82 | A |
| ATOM | 4470 | OE1 | GLN | A | 567 | 49.683 | 67.581 | −25.238 | 1.00 | 17.65 | A |
| ATOM | 4471 | NE2 | GLN | A | 567 | 50.198 | 69.674 | −25.868 | 1.00 | 19.30 | A |
| ATOM | 4472 | C | GLN | A | 567 | 50.944 | 68.709 | −30.090 | 1.00 | 15.02 | A |
| ATOM | 4473 | O | GLN | A | 567 | 52.130 | 69.053 | −30.065 | 1.00 | 14.23 | A |
| ATOM | 4474 | N | LEU | A | 568 | 49.966 | 69.537 | −30.429 | 1.00 | 14.97 | A |
| ATOM | 4475 | CA | LEU | A | 568 | 50.278 | 70.922 | −30.735 | 1.00 | 13.72 | A |
| ATOM | 4476 | CB | LEU | A | 568 | 49.096 | 71.624 | −31.410 | 1.00 | 15.18 | A |
| ATOM | 4477 | CG | LEU | A | 568 | 48.672 | 71.202 | −32.821 | 1.00 | 14.75 | A |
| ATOM | 4478 | CD1 | LEU | A | 568 | 47.656 | 72.209 | −33.352 | 1.00 | 15.08 | A |
| ATOM | 4479 | CD2 | LEU | A | 568 | 49.885 | 71.161 | −33.742 | 1.00 | 15.38 | A |
| ATOM | 4480 | C | LEU | A | 568 | 50.585 | 71.616 | −29.413 | 1.00 | 13.98 | A |
| ATOM | 4481 | O | LEU | A | 568 | 49.939 | 71.354 | −28.398 | 1.00 | 12.88 | A |
| ATOM | 4482 | N | VAL | A | 569 | 51.589 | 72.484 | −29.427 | 1.00 | 13.42 | A |
| ATOM | 4483 | CA | VAL | A | 569 | 51.969 | 73.244 | −28.246 | 1.00 | 14.61 | A |
| ATOM | 4484 | CB | VAL | A | 569 | 53.335 | 72.776 | −27.681 | 1.00 | 14.13 | A |
| ATOM | 4485 | CG1 | VAL | A | 569 | 53.202 | 71.370 | −27.095 | 1.00 | 14.34 | A |
| ATOM | 4486 | CG2 | VAL | A | 569 | 54.389 | 72.791 | −28.781 | 1.00 | 14.63 | A |
| ATOM | 4487 | C | VAL | A | 569 | 52.070 | 74.704 | −28.676 | 1.00 | 14.28 | A |
| ATOM | 4488 | O | VAL | A | 569 | 52.362 | 74.988 | −29.833 | 1.00 | 14.20 | A |
| ATOM | 4489 | N | ASP | A | 570 | 51.804 | 75.629 | −27.762 | 1.00 | 14.70 | A |
| ATOM | 4490 | CA | ASP | A | 570 | 51.894 | 77.041 | −28.107 | 1.00 | 15.22 | A |
| ATOM | 4491 | CB | ASP | A | 570 | 50.491 | 77.650 | −28.263 | 1.00 | 18.14 | A |
| ATOM | 4492 | CG | ASP | A | 570 | 49.738 | 77.751 | −26.948 | 1.00 | 20.68 | A |
| ATOM | 4493 | OD1 | ASP | A | 570 | 49.536 | 76.714 | −26.288 | 1.00 | 23.52 | A |
| ATOM | 4494 | OD2 | ASP | A | 570 | 49.343 | 78.874 | −26.577 | 1.00 | 23.31 | A |
| ATOM | 4495 | C | ASP | A | 570 | 52.699 | 77.818 | −27.075 | 1.00 | 14.70 | A |
| ATOM | 4496 | O | ASP | A | 570 | 52.799 | 77.408 | −25.915 | 1.00 | 13.72 | A |
| ATOM | 4497 | N | PHE | A | 571 | 53.304 | 78.916 | −27.520 | 1.00 | 13.84 | A |
| ATOM | 4498 | CA | PHE | A | 571 | 54.096 | 79.784 | −26.651 | 1.00 | 14.16 | A |
| ATOM | 4499 | CB | PHE | A | 571 | 55.602 | 79.556 | −26.833 | 1.00 | 13.63 | A |
| ATOM | 4500 | CG | PHE | A | 571 | 56.069 | 78.181 | −26.470 | 1.00 | 13.26 | A |
| ATOM | 4501 | CD1 | PHE | A | 571 | 56.017 | 77.148 | −27.398 | 1.00 | 13.24 | A |
| ATOM | 4502 | CD2 | PHE | A | 571 | 56.576 | 77.922 | −25.201 | 1.00 | 13.50 | A |
| ATOM | 4503 | CE1 | PHE | A | 571 | 56.468 | 75.869 | −27.066 | 1.00 | 13.34 | A |
| ATOM | 4504 | CE2 | PHE | A | 571 | 57.027 | 76.645 | −24.860 | 1.00 | 13.94 | A |
| ATOM | 4505 | CZ | PHE | A | 571 | 56.972 | 75.620 | −25.797 | 1.00 | 13.21 | A |
| ATOM | 4506 | C | PHE | A | 571 | 53.815 | 81.226 | −27.040 | 1.00 | 14.55 | A |
| ATOM | 4507 | O | PHE | A | 571 | 53.459 | 81.502 | −28.185 | 1.00 | 14.25 | A |
| ATOM | 4508 | N | TYR | A | 572 | 53.973 | 82.141 | −26.089 | 1.00 | 14.89 | A |
| ATOM | 4509 | CA | TYR | A | 572 | 53.792 | 83.560 | −26.376 | 1.00 | 15.36 | A |
| ATOM | 4510 | CB | TYR | A | 572 | 53.373 | 84.337 | −25.122 | 1.00 | 16.02 | A |
| ATOM | 4511 | CG | TYR | A | 572 | 51.938 | 84.151 | −24.681 | 1.00 | 18.23 | A |
| ATOM | 4512 | CD1 | TYR | A | 572 | 51.021 | 83.456 | −25.468 | 1.00 | 18.10 | A |
| ATOM | 4513 | CE1 | TYR | A | 572 | 49.692 | 83.314 | −25.072 | 1.00 | 19.65 | A |
| ATOM | 4514 | CD2 | TYR | A | 572 | 51.489 | 84.702 | −23.479 | 1.00 | 19.53 | A |
| ATOM | 4515 | CE2 | TYR | A | 572 | 50.162 | 84.569 | −23.075 | 1.00 | 20.24 | A |
| ATOM | 4516 | CZ | TYR | A | 572 | 49.270 | 83.874 | −23.876 | 1.00 | 20.05 | A |
| ATOM | 4517 | OH | TYR | A | 572 | 47.956 | 83.742 | −23.482 | 1.00 | 20.96 | A |
| ATOM | 4518 | C | TYR | A | 572 | 55.149 | 84.084 | −26.832 | 1.00 | 15.16 | A |
| ATOM | 4519 | O | TYR | A | 572 | 56.165 | 83.783 | −26.206 | 1.00 | 15.39 | A |
| ATOM | 4520 | N | VAL | A | 573 | 55.166 | 84.857 | −27.916 | 1.00 | 15.22 | A |
| ATOM | 4521 | CA | VAL | A | 573 | 56.405 | 85.436 | −28.439 | 1.00 | 14.74 | A |
| ATOM | 4522 | CB | VAL | A | 573 | 56.841 | 84.759 | −29.763 | 1.00 | 15.15 | A |
| ATOM | 4523 | CG1 | VAL | A | 573 | 57.300 | 83.327 | −29.493 | 1.00 | 14.52 | A |
| ATOM | 4524 | CG2 | VAL | A | 573 | 55.690 | 84.765 | −30.754 | 1.00 | 14.39 | A |
| ATOM | 4525 | C | VAL | A | 573 | 56.213 | 86.938 | −28.683 | 1.00 | 15.63 | A |
| ATOM | 4526 | O | VAL | A | 573 | 55.102 | 87.390 | −28.943 | 1.00 | 15.28 | A |
| ATOM | 4527 | N | SER | A | 574 | 57.298 | 87.704 | −28.602 | 1.00 | 16.43 | A |
| ATOM | 4528 | CA | SER | A | 574 | 57.233 | 89.156 | −28.783 | 1.00 | 17.10 | A |
| ATOM | 4529 | CB | SER | A | 574 | 58.410 | 89.824 | −28.067 | 1.00 | 16.27 | A |
| ATOM | 4530 | OG | SER | A | 574 | 59.639 | 89.471 | −28.675 | 1.00 | 15.14 | A |
| ATOM | 4531 | C | SER | A | 574 | 57.206 | 89.609 | −30.241 | 1.00 | 18.36 | A |
| ATOM | 4532 | O | SER | A | 574 | 57.258 | 90.808 | −30.525 | 1.00 | 18.70 | A |
| ATOM | 4533 | N | SER | A | 575 | 57.138 | 88.655 | −31.162 | 1.00 | 18.80 | A |
| ATOM | 4534 | CA | SER | A | 575 | 57.088 | 88.968 | −32.584 | 1.00 | 19.82 | A |
| ATOM | 4535 | CB | SER | A | 575 | 58.496 | 89.096 | −33.166 | 1.00 | 20.11 | A |
| ATOM | 4536 | OG | SER | A | 575 | 58.438 | 89.191 | −34.582 | 1.00 | 19.88 | A |
| ATOM | 4537 | C | SER | A | 575 | 56.345 | 87.885 | −33.343 | 1.00 | 19.97 | A |
| ATOM | 4538 | O | SER | A | 575 | 56.423 | 86.708 | −32.997 | 1.00 | 20.29 | A |
| ATOM | 4539 | N | PRO | A | 576 | 55.601 | 88.272 | −34.387 | 1.00 | 20.08 | A |
| ATOM | 4540 | CD | PRO | A | 576 | 55.277 | 89.649 | −34.809 | 1.00 | 20.22 | A |
| ATOM | 4541 | CA | PRO | A | 576 | 54.856 | 87.290 | −35.179 | 1.00 | 19.56 | A |
| ATOM | 4542 | CB | PRO | A | 576 | 53.800 | 88.146 | −35.866 | 1.00 | 20.22 | A |
| ATOM | 4543 | CG | PRO | A | 576 | 54.552 | 89.428 | −36.125 | 1.00 | 20.64 | A |
| ATOM | 4544 | C | PRO | A | 576 | 55.785 | 86.602 | −36.177 | 1.00 | 19.20 | A |
| ATOM | 4545 | O | PRO | A | 576 | 55.448 | 85.566 | −36.741 | 1.00 | 19.27 | A |
| ATOM | 4546 | N | PHE | A | 577 | 56.961 | 87.187 | −36.380 | 1.00 | 18.89 | A |
| ATOM | 4547 | CA | PHE | A | 577 | 57.938 | 86.652 | −37.321 | 1.00 | 18.98 | A |
| ATOM | 4548 | CB | PHE | A | 577 | 58.699 | 87.807 | −37.969 | 1.00 | 20.18 | A |
| ATOM | 4549 | CG | PHE | A | 577 | 57.805 | 88.800 | −38.658 | 1.00 | 21.35 | A |
| ATOM | 4550 | CD1 | PHE | A | 577 | 58.050 | 90.166 | −38.554 | 1.00 | 22.43 | A |
| ATOM | 4551 | CD2 | PHE | A | 577 | 56.711 | 88.369 | −39.404 | 1.00 | 21.36 | A |
| ATOM | 4552 | CE1 | PHE | A | 577 | 57.216 | 91.091 | −39.183 | 1.00 | 23.04 | A |
| ATOM | 4553 | CE2 | PHE | A | 577 | 55.870 | 89.286 | −40.038 | 1.00 | 22.22 | A |
| ATOM | 4554 | CZ | PHE | A | 577 | 56.125 | 90.650 | −39.925 | 1.00 | 22.87 | A |
| ATOM | 4555 | C | PHE | A | 577 | 58.907 | 85.689 | −36.643 | 1.00 | 18.67 | A |
| ATOM | 4556 | O | PHE | A | 577 | 60.089 | 85.993 | −36.463 | 1.00 | 18.03 | A |
| ATOM | 4557 | N | VAL | A | 578 | 58.387 | 84.521 | −36.280 | 1.00 | 18.09 | A |
| ATOM | 4558 | CA | VAL | A | 578 | 59.170 | 83.495 | −35.607 | 1.00 | 18.08 | A |
| ATOM | 4559 | CB | VAL | A | 578 | 58.705 | 83.330 | −34.135 | 1.00 | 17.94 | A |
| ATOM | 4560 | CG1 | VAL | A | 578 | 59.526 | 82.252 | −33.435 | 1.00 | 17.87 | A |
| ATOM | 4561 | CG2 | VAL | A | 578 | 58.832 | 84.660 | −33.404 | 1.00 | 18.13 | A |
| ATOM | 4562 | C | VAL | A | 578 | 59.023 | 82.159 | −36.326 | 1.00 | 17.84 | A |
| ATOM | 4563 | O | VAL | A | 578 | 57.937 | 81.808 | −36.786 | 1.00 | 17.94 | A |
| ATOM | 4564 | N | SER | A | 579 | 60.123 | 81.422 | −36.428 | 1.00 | 18.30 | A |
| ATOM | 4565 | CA | SER | A | 579 | 60.110 | 80.120 | −37.081 | 1.00 | 18.55 | A |
| ATOM | 4566 | CB | SER | A | 579 | 60.967 | 80.145 | −38.353 | 1.00 | 19.76 | A |
| ATOM | 4567 | OG | SER | A | 579 | 62.307 | 80.502 | −38.069 | 1.00 | 22.42 | A |
| ATOM | 4568 | C | SER | A | 579 | 60.627 | 79.065 | −36.110 | 1.00 | 17.81 | A |
| ATOM | 4569 | O | SER | A | 579 | 61.420 | 79.363 | −35.218 | 1.00 | 18.62 | A |
| ATOM | 4570 | N | VAL | A | 580 | 60.173 | 77.832 | −36.292 | 1.00 | 16.54 | A |
| ATOM | 4571 | CA | VAL | A | 580 | 60.558 | 76.732 | −35.423 | 1.00 | 16.12 | A |
| ATOM | 4572 | CB | VAL | A | 580 | 59.304 | 76.030 | −34.851 | 1.00 | 16.04 | A |
| ATOM | 4573 | CG1 | VAL | A | 580 | 59.706 | 74.975 | −33.825 | 1.00 | 15.88 | A |
| ATOM | 4574 | CG2 | VAL | A | 580 | 58.374 | 77.059 | −34.231 | 1.00 | 14.55 | A |
| ATOM | 4575 | C | VAL | A | 580 | 61.400 | 75.685 | −36.148 | 1.00 | 16.46 | A |
| ATOM | 4576 | O | VAL | A | 580 | 61.194 | 75.413 | −37.331 | 1.00 | 16.12 | A |
| ATOM | 4577 | N | THR | A | 581 | 62.348 | 75.109 | −35.416 | 1.00 | 16.83 | A |
| ATOM | 4578 | CA | THR | A | 581 | 63.231 | 74.063 | −35.926 | 1.00 | 17.81 | A |
| ATOM | 4579 | CB | THR | A | 581 | 64.600 | 74.630 | −36.369 | 1.00 | 17.60 | A |
| ATOM | 4580 | OG1 | THR | A | 581 | 65.180 | 75.374 | −35.289 | 1.00 | 17.59 | A |
| ATOM | 4581 | CG2 | THR | A | 581 | 64.450 | 75.535 | −37.592 | 1.00 | 17.67 | A |
| ATOM | 4582 | C | THR | A | 581 | 63.491 | 73.086 | −34.780 | 1.00 | 18.67 | A |
| ATOM | 4583 | O | THR | A | 581 | 63.366 | 73.458 | −33.611 | 1.00 | 17.83 | A |
| ATOM | 4584 | N | ASP | A | 582 | 63.830 | 71.839 | −35.105 | 1.00 | 19.42 | A |
| ATOM | 4585 | CA | ASP | A | 582 | 64.150 | 70.870 | −34.063 | 1.00 | 20.83 | A |
| ATOM | 4586 | CB | ASP | A | 582 | 63.734 | 69.448 | −34.475 | 1.00 | 20.66 | A |
| ATOM | 4587 | CG | ASP | A | 582 | 64.403 | 68.966 | −35.751 | 1.00 | 20.65 | A |
| ATOM | 4588 | OD1 | ASP | A | 582 | 63.887 | 67.988 | −36.337 | 1.00 | 21.09 | A |
| ATOM | 4589 | OD2 | ASP | A | 582 | 65.434 | 69.536 | −36.164 | 1.00 | 18.90 | A |
| ATOM | 4590 | C | ASP | A | 582 | 65.658 | 70.992 | −33.842 | 1.00 | 22.10 | A |
| ATOM | 4591 | O | ASP | A | 582 | 66.294 | 71.843 | −34.460 | 1.00 | 21.76 | A |
| ATOM | 4592 | N | LEU | A | 583 | 66.252 | 70.173 | −32.981 | 1.00 | 23.97 | A |
| ATOM | 4593 | CA | LEU | A | 583 | 67.682 | 70.345 | −32.759 | 1.00 | 25.53 | A |
| ATOM | 4594 | CB | LEU | A | 583 | 68.141 | 69.642 | −31.482 | 1.00 | 27.13 | A |
| ATOM | 4595 | CG | LEU | A | 583 | 69.400 | 70.340 | −30.953 | 1.00 | 27.21 | A |
| ATOM | 4596 | CD1 | LEU | A | 583 | 69.045 | 71.761 | −30.536 | 1.00 | 27.75 | A |
| ATOM | 4597 | CD2 | LEU | A | 583 | 69.984 | 69.584 | −29.789 | 1.00 | 28.89 | A |
| ATOM | 4598 | C | LEU | A | 583 | 68.566 | 69.923 | −33.926 | 1.00 | 25.69 | A |
| ATOM | 4599 | O | LEU | A | 583 | 69.760 | 70.217 | −33.934 | 1.00 | 26.03 | A |
| ATOM | 4600 | N | ALA | A | 584 | 67.987 | 69.238 | −34.909 | 1.00 | 25.08 | A |
| ATOM | 4601 | CA | ALA | A | 584 | 68.746 | 68.822 | −36.085 | 1.00 | 24.31 | A |
| ATOM | 4602 | CB | ALA | A | 584 | 68.185 | 67.525 | −36.648 | 1.00 | 24.53 | A |
| ATOM | 4603 | C | ALA | A | 584 | 68.630 | 69.942 | −37.114 | 1.00 | 24.07 | A |
| ATOM | 4604 | O | ALA | A | 584 | 69.059 | 69.809 | −38.263 | 1.00 | 23.07 | A |
| ATOM | 4605 | N | ASN | A | 585 | 68.039 | 71.049 | −36.678 | 1.00 | 23.90 | A |
| ATOM | 4606 | CA | ASN | A | 585 | 67.838 | 72.221 | −37.517 | 1.00 | 24.16 | A |
| ATOM | 4607 | CB | ASN | A | 585 | 69.160 | 72.666 | −38.148 | 1.00 | 26.50 | A |
| ATOM | 4608 | CG | ASN | A | 585 | 69.451 | 74.132 | −37.906 | 1.00 | 28.18 | A |
| ATOM | 4609 | OD1 | ASN | A | 585 | 68.570 | 74.986 | −38.038 | 1.00 | 28.37 | A |
| ATOM | 4610 | ND2 | ASN | A | 585 | 70.695 | 74.435 | −37.555 | 1.00 | 29.91 | A |
| ATOM | 4611 | C | ASN | A | 585 | 66.805 | 71.987 | −38.614 | 1.00 | 23.60 | A |
| ATOM | 4612 | O | ASN | A | 585 | 66.774 | 72.719 | −39.604 | 1.00 | 23.48 | A |
| ATOM | 4613 | N | ASN | A | 586 | 65.970 | 70.965 | −38.451 | 1.00 | 21.95 | A |
| ATOM | 4614 | CA | ASN | A | 586 | 64.931 | 70.690 | −39.435 | 1.00 | 21.75 | A |
| ATOM | 4615 | CB | ASN | A | 586 | 64.356 | 69.280 | −39.281 | 1.00 | 22.27 | A |
| ATOM | 4616 | CG | ASN | A | 586 | 65.392 | 68.199 | −39.440 | 1.00 | 23.42 | A |
| ATOM | 4617 | OD1 | ASN | A | 586 | 66.173 | 68.203 | −40.391 | 1.00 | 23.15 | A |
| ATOM | 4618 | ND2 | ASN | A | 586 | 65.393 | 67.244 | −38.511 | 1.00 | 22.98 | A |
| ATOM | 4619 | C | ASN | A | 586 | 63.797 | 71.674 | −39.190 | 1.00 | 21.19 | A |
| ATOM | 4620 | O | ASN | A | 586 | 63.369 | 71.861 | −38.053 | 1.00 | 20.23 | A |
| ATOM | 4621 | N | PRO | A | 587 | 63.296 | 72.318 | −40.250 | 1.00 | 20.95 | A |
| ATOM | 4622 | CD | PRO | A | 587 | 63.704 | 72.279 | −41.666 | 1.00 | 21.61 | A |
| ATOM | 4623 | CA | PRO | A | 587 | 62.197 | 73.264 | −40.042 | 1.00 | 20.35 | A |
| ATOM | 4624 | CB | PRO | A | 587 | 62.034 | 73.914 | −41.417 | 1.00 | 21.29 | A |
| ATOM | 4625 | CG | PRO | A | 587 | 62.488 | 72.839 | −42.360 | 1.00 | 21.88 | A |
| ATOM | 4626 | C | PRO | A | 587 | 60.943 | 72.525 | −39.583 | 1.00 | 19.89 | A |
| ATOM | 4627 | O | PRO | A | 587 | 60.727 | 71.366 | −39.937 | 1.00 | 19.71 | A |
| ATOM | 4628 | N | VAL | A | 588 | 60.132 | 73.195 | −38.773 | 1.00 | 18.78 | A |
| ATOM | 4629 | CA | VAL | A | 588 | 58.897 | 72.615 | −38.258 | 1.00 | 18.35 | A |
| ATOM | 4630 | CB | VAL | A | 588 | 58.953 | 72.452 | −36.721 | 1.00 | 18.72 | A |
| ATOM | 4631 | CG1 | VAL | A | 588 | 57.621 | 71.936 | −36.202 | 1.00 | 18.58 | A |
| ATOM | 4632 | CG2 | VAL | A | 588 | 60.079 | 71.499 | −36.338 | 1.00 | 17.83 | A |
| ATOM | 4633 | C | VAL | A | 588 | 57.777 | 73.582 | −38.617 | 1.00 | 18.17 | A |
| ATOM | 4634 | O | VAL | A | 588 | 57.871 | 74.774 | −38.325 | 1.00 | 17.36 | A |
| ATOM | 4635 | N | GLU | A | 589 | 56.727 | 73.079 | −39.259 | 1.00 | 18.10 | A |
| ATOM | 4636 | CA | GLU | A | 589 | 55.623 | 73.947 | −39.646 | 1.00 | 18.70 | A |
| ATOM | 4637 | CB | GLU | A | 589 | 54.571 | 73.176 | −40.441 | 1.00 | 20.53 | A |
| ATOM | 4638 | CG | GLU | A | 589 | 53.667 | 74.093 | −41.248 | 1.00 | 24.87 | A |
| ATOM | 4639 | CD | GLU | A | 589 | 52.450 | 73.389 | −41.801 | 1.00 | 27.02 | A |
| ATOM | 4640 | OE1 | GLU | A | 589 | 52.585 | 72.232 | −42.254 | 1.00 | 28.61 | A |
| ATOM | 4641 | OE2 | GLU | A | 589 | 51.357 | 73.999 | −41.792 | 1.00 | 28.42 | A |
| ATOM | 4642 | C | GLU | A | 589 | 54.978 | 74.556 | −38.410 | 1.00 | 17.96 | A |
| ATOM | 4643 | O | GLU | A | 589 | 54.703 | 73.860 | −37.434 | 1.00 | 16.94 | A |
| ATOM | 4644 | N | ALA | A | 590 | 54.733 | 75.861 | −38.464 | 1.00 | 16.87 | A |
| ATOM | 4645 | CA | ALA | A | 590 | 54.134 | 76.569 | −37.342 | 1.00 | 16.73 | A |
| ATOM | 4646 | CB | ALA | A | 590 | 55.209 | 77.342 | −36.589 | 1.00 | 17.08 | A |
| ATOM | 4647 | C | ALA | A | 590 | 53.041 | 77.522 | −37.805 | 1.00 | 16.62 | A |
| ATOM | 4648 | O | ALA | A | 590 | 52.951 | 77.857 | −38.985 | 1.00 | 16.45 | A |
| ATOM | 4649 | N | GLN | A | 591 | 52.211 | 77.946 | −36.860 | 1.00 | 16.54 | A |
| ATOM | 4650 | CA | GLN | A | 591 | 51.120 | 78.876 | −37.125 | 1.00 | 16.07 | A |
| ATOM | 4651 | CB | GLN | A | 591 | 49.766 | 78.159 | −37.076 | 1.00 | 16.36 | A |
| ATOM | 4652 | CG | GLN | A | 591 | 48.563 | 79.105 | −37.142 | 1.00 | 15.76 | A |
| ATOM | 4653 | CD | GLN | A | 591 | 47.238 | 78.402 | −36.876 | 1.00 | 15.84 | A |
| ATOM | 4654 | OE1 | GLN | A | 591 | 46.964 | 77.337 | −37.430 | 1.00 | 15.89 | A |
| ATOM | 4655 | NE2 | GLN | A | 591 | 46.407 | 79.003 | −36.030 | 1.00 | 14.94 | A |
| ATOM | 4656 | C | GLN | A | 591 | 51.142 | 79.938 | −36.038 | 1.00 | 16.10 | A |
| ATOM | 4657 | O | GLN | A | 591 | 51.308 | 79.624 | −34.857 | 1.00 | 16.16 | A |
| ATOM | 4658 | N | VAL | A | 592 | 50.987 | 81.196 | −36.429 | 1.00 | 15.49 | A |
| ATOM | 4659 | CA | VAL | A | 592 | 50.959 | 82.268 | −35.449 | 1.00 | 15.45 | A |
| ATOM | 4660 | CB | VAL | A | 592 | 51.977 | 83.382 | −35.785 | 1.00 | 15.57 | A |
| ATOM | 4661 | CG1 | VAL | A | 592 | 51.755 | 84.588 | −34.869 | 1.00 | 15.47 | A |
| ATOM | 4662 | CG2 | VAL | A | 592 | 53.398 | 82.850 | −35.599 | 1.00 | 15.57 | A |
| ATOM | 4663 | C | VAL | A | 592 | 49.551 | 82.841 | −35.412 | 1.00 | 15.66 | A |
| ATOM | 4664 | O | VAL | A | 592 | 48.917 | 83.029 | −36.450 | 1.00 | 16.23 | A |
| ATOM | 4665 | N | SER | A | 593 | 49.057 | 83.078 | −34.205 | 1.00 | 15.93 | A |
| ATOM | 4666 | CA | SER | A | 593 | 47.728 | 83.639 | −34.001 | 1.00 | 16.58 | A |
| ATOM | 4667 | CB | SER | A | 593 | 46.768 | 82.583 | −33.434 | 1.00 | 16.33 | A |
| ATOM | 4668 | OG | SER | A | 593 | 46.591 | 81.493 | −34.328 | 1.00 | 17.88 | A |
| ATOM | 4669 | C | SER | A | 593 | 47.868 | 84.775 | −33.003 | 1.00 | 16.55 | A |
| ATOM | 4670 | O | SER | A | 593 | 48.875 | 84.874 | −32.296 | 1.00 | 17.21 | A |
| ATOM | 4671 | N | PRO | A | 594 | 46.864 | 85.657 | −32.931 | 1.00 | 16.44 | A |
| ATOM | 4672 | CD | PRO | A | 594 | 45.738 | 85.835 | −33.863 | 1.00 | 16.46 | A |
| ATOM | 4673 | CA | PRO | A | 594 | 46.941 | 86.771 | −31.983 | 1.00 | 16.18 | A |
| ATOM | 4674 | CB | PRO | A | 594 | 45.831 | 87.713 | −32.453 | 1.00 | 16.54 | A |
| ATOM | 4675 | CG | PRO | A | 594 | 45.607 | 87.332 | −33.898 | 1.00 | 16.24 | A |
| ATOM | 4676 | C | PRO | A | 594 | 46.676 | 86.288 | −30.561 | 1.00 | 16.50 | A |
| ATOM | 4677 | O | PRO | A | 594 | 46.239 | 85.153 | −30.350 | 1.00 | 16.26 | A |
| ATOM | 4678 | N | VAL | A | 595 | 46.955 | 87.147 | −29.587 | 1.00 | 15.96 | A |
| ATOM | 4679 | CA | VAL | A | 595 | 46.672 | 86.821 | −28.199 | 1.00 | 16.67 | A |
| ATOM | 4680 | CB | VAL | A | 595 | 47.782 | 87.306 | −27.241 | 1.00 | 16.47 | A |
| ATOM | 4681 | CG1 | VAL | A | 595 | 47.348 | 87.080 | −25.792 | 1.00 | 16.20 | A |
| ATOM | 4682 | CG2 | VAL | A | 595 | 49.071 | 86.551 | −27.516 | 1.00 | 17.31 | A |
| ATOM | 4683 | C | VAL | A | 595 | 45.382 | 87.572 | −27.895 | 1.00 | 16.73 | A |
| ATOM | 4684 | O | VAL | A | 595 | 45.386 | 88.794 | −27.765 | 1.00 | 17.05 | A |
| ATOM | 4685 | N | TRP | A | 596 | 44.277 | 86.838 | −27.809 | 1.00 | 16.70 | A |
| ATOM | 4686 | CA | TRP | A | 596 | 42.979 | 87.441 | −27.541 | 1.00 | 17.84 | A |
| ATOM | 4687 | CB | TRP | A | 596 | 41.890 | 86.779 | −28.400 | 1.00 | 17.13 | A |
| ATOM | 4688 | CG | TRP | A | 596 | 42.053 | 86.944 | −29.878 | 1.00 | 17.49 | A |
| ATOM | 4689 | CD2 | TRP | A | 596 | 41.740 | 88.111 | −30.649 | 1.00 | 17.58 | A |
| ATOM | 4690 | CE2 | TRP | A | 596 | 42.029 | 87.810 | −31.999 | 1.00 | 17.37 | A |
| ATOM | 4691 | CE3 | TRP | A | 596 | 41.243 | 89.383 | −30.329 | 1.00 | 18.00 | A |
| ATOM | 4692 | CD1 | TRP | A | 596 | 42.509 | 86.010 | −30.766 | 1.00 | 17.26 | A |
| ATOM | 4693 | NE1 | TRP | A | 596 | 42.494 | 86.521 | −32.042 | 1.00 | 17.27 | A |
| ATOM | 4694 | CZ2 | TRP | A | 596 | 41.837 | 88.734 | −33.031 | 1.00 | 18.13 | A |
| ATOM | 4695 | CZ3 | TRP | A | 596 | 41.050 | 90.305 | −31.359 | 1.00 | 18.36 | A |
| ATOM | 4696 | CH2 | TRP | A | 596 | 41.348 | 89.974 | −32.692 | 1.00 | 18.70 | A |
| ATOM | 4697 | C | TRP | A | 596 | 42.569 | 87.329 | −26.077 | 1.00 | 18.84 | A |
| ATOM | 4698 | O | TRP | A | 596 | 42.689 | 86.267 | −25.466 | 1.00 | 17.93 | A |
| ATOM | 4699 | N | SER | A | 597 | 42.084 | 88.434 | −25.519 | 1.00 | 19.67 | A |
| ATOM | 4700 | CA | SER | A | 597 | 41.620 | 88.451 | −24.137 | 1.00 | 21.40 | A |
| ATOM | 4701 | CB | SER | A | 597 | 42.561 | 89.280 | −23.261 | 1.00 | 21.75 | A |
| ATOM | 4702 | OG | SER | A | 597 | 42.699 | 90.589 | −23.774 | 1.00 | 24.13 | A |
| ATOM | 4703 | C | SER | A | 597 | 40.221 | 89.055 | −24.127 | 1.00 | 21.80 | A |
| ATOM | 4704 | O | SER | A | 597 | 39.973 | 90.082 | −24.764 | 1.00 | 22.44 | A |
| ATOM | 4705 | N | TRP | A | 598 | 39.306 | 88.411 | −23.414 | 1.00 | 22.12 | A |
| ATOM | 4706 | CA | TRP | A | 598 | 37.934 | 88.888 | −23.343 | 1.00 | 23.28 | A |
| ATOM | 4707 | CB | TRP | A | 598 | 36.967 | 87.704 | −23.289 | 1.00 | 21.46 | A |
| ATOM | 4708 | CG | TRP | A | 598 | 36.940 | 86.928 | −24.570 | 1.00 | 19.87 | A |
| ATOM | 4709 | CD2 | TRP | A | 598 | 35.925 | 86.976 | −25.577 | 1.00 | 19.13 | A |
| ATOM | 4710 | CE2 | TRP | A | 598 | 36.334 | 86.122 | −26.627 | 1.00 | 18.71 | A |
| ATOM | 4711 | CE3 | TRP | A | 598 | 34.708 | 87.662 | −25.698 | 1.00 | 19.08 | A |
| ATOM | 4712 | CD1 | TRP | A | 598 | 37.898 | 86.070 | −25.033 | 1.00 | 19.36 | A |
| ATOM | 4713 | NE1 | TRP | A | 598 | 37.540 | 85.581 | −26.269 | 1.00 | 18.19 | A |
| ATOM | 4714 | CZ2 | TRP | A | 598 | 35.569 | 85.934 | −27.782 | 1.00 | 18.54 | A |
| ATOM | 4715 | CZ3 | TRP | A | 598 | 33.947 | 87.475 | −26.849 | 1.00 | 18.32 | A |
| ATOM | 4716 | CH2 | TRP | A | 598 | 34.382 | 86.617 | −27.876 | 1.00 | 18.60 | A |
| ATOM | 4717 | C | TRP | A | 598 | 37.718 | 89.802 | −22.149 | 1.00 | 25.15 | A |
| ATOM | 4718 | O | TRP | A | 598 | 38.204 | 89.536 | −21.051 | 1.00 | 25.24 | A |
| ATOM | 4719 | N | HIS | A | 599 | 36.982 | 90.883 | −22.374 | 1.00 | 27.74 | A |
| ATOM | 4720 | CA | HIS | A | 599 | 36.728 | 91.848 | −21.319 | 1.00 | 30.86 | A |
| ATOM | 4721 | CB | HIS | A | 599 | 37.605 | 93.081 | −21.542 | 1.00 | 32.48 | A |
| ATOM | 4722 | CG | HIS | A | 599 | 39.068 | 92.770 | −21.604 | 1.00 | 34.54 | A |
| ATOM | 4723 | CD2 | HIS | A | 599 | 39.934 | 92.763 | −22.645 | 1.00 | 35.10 | A |
| ATOM | 4724 | ND1 | HIS | A | 599 | 39.793 | 92.372 | −20.500 | 1.00 | 35.25 | A |
| ATOM | 4725 | CE1 | HIS | A | 599 | 41.042 | 92.135 | −20.859 | 1.00 | 35.53 | A |
| ATOM | 4726 | NE2 | HIS | A | 599 | 41.154 | 92.364 | −22.156 | 1.00 | 35.52 | A |
| ATOM | 4727 | C | HIS | A | 599 | 35.271 | 92.268 | −21.242 | 1.00 | 32.18 | A |
| ATOM | 4728 | O | HIS | A | 599 | 34.606 | 92.441 | −22.264 | 1.00 | 31.64 | A |
| ATOM | 4729 | N | HIS | A | 600 | 34.779 | 92.420 | −20.019 | 1.00 | 34.30 | A |
| ATOM | 4730 | CA | HIS | A | 600 | 33.407 | 92.846 | −19.803 | 1.00 | 36.52 | A |
| ATOM | 4731 | CB | HIS | A | 600 | 32.895 | 92.352 | −18.449 | 1.00 | 38.40 | A |
| ATOM | 4732 | CG | HIS | A | 600 | 31.437 | 92.609 | −18.225 | 1.00 | 40.80 | A |
| ATOM | 4733 | CD2 | HIS | A | 600 | 30.432 | 91.782 | −17.852 | 1.00 | 41.76 | A |
| ATOM | 4734 | ND1 | HIS | A | 600 | 30.868 | 93.855 | −18.384 | 1.00 | 41.71 | A |
| ATOM | 4735 | CE1 | HIS | A | 600 | 29.575 | 93.784 | −18.120 | 1.00 | 42.34 | A |
| ATOM | 4736 | NE2 | HIS | A | 600 | 29.285 | 92.537 | −17.794 | 1.00 | 42.76 | A |
| ATOM | 4737 | C | HIS | A | 600 | 33.452 | 94.366 | −19.820 | 1.00 | 36.92 | A |
| ATOM | 4738 | O | HIS | A | 600 | 33.792 | 94.996 | −18.820 | 1.00 | 37.27 | A |
| ATOM | 4739 | N | ASP | A | 601 | 33.128 | 94.948 | −20.969 | 1.00 | 37.29 | A |
| ATOM | 4740 | CA | ASP | A | 601 | 33.150 | 96.395 | −21.125 | 1.00 | 37.97 | A |
| ATOM | 4741 | CB | ASP | A | 601 | 32.959 | 96.763 | −22.597 | 1.00 | 38.10 | A |
| ATOM | 4742 | CG | ASP | A | 601 | 33.355 | 98.194 | −22.895 | 1.00 | 38.67 | A |
| ATOM | 4743 | OD1 | ASP | A | 601 | 32.854 | 99.111 | −22.211 | 1.00 | 38.47 | A |
| ATOM | 4744 | OD2 | ASP | A | 601 | 34.167 | 98.403 | −23.819 | 1.00 | 39.33 | A |
| ATOM | 4745 | C | ASP | A | 601 | 32.062 | 97.056 | −20.280 | 1.00 | 38.23 | A |
| ATOM | 4746 | O | ASP | A | 601 | 30.881 | 97.001 | −20.619 | 1.00 | 37.93 | A |
| ATOM | 4747 | N | THR | A | 602 | 32.474 | 97.682 | −19.181 | 1.00 | 38.57 | A |
| ATOM | 4748 | CA | THR | A | 602 | 31.548 | 98.357 | −18.278 | 1.00 | 39.06 | A |
| ATOM | 4749 | CB | THR | A | 602 | 32.284 | 98.918 | −17.040 | 1.00 | 39.91 | A |
| ATOM | 4750 | OG1 | THR | A | 602 | 32.925 | 97.847 | −16.337 | 1.00 | 41.22 | A |
| ATOM | 4751 | CG2 | THR | A | 602 | 31.302 | 99.616 | −16.103 | 1.00 | 40.79 | A |
| ATOM | 4752 | C | THR | A | 602 | 30.826 | 99.510 | −18.965 | 1.00 | 38.24 | A |
| ATOM | 4753 | O | THR | A | 602 | 29.726 | 99.887 | −18.565 | 1.00 | 38.57 | A |
| ATOM | 4754 | N | LEU | A | 603 | 31.448 | 100.063 | −20.001 | 1.00 | 37.19 | A |
| ATOM | 4755 | CA | LEU | A | 603 | 30.869 | 101.184 | −20.733 | 1.00 | 36.14 | A |
| ATOM | 4756 | CB | LEU | A | 603 | 31.974 | 102.006 | −21.408 | 1.00 | 37.37 | A |
| ATOM | 4757 | CG | LEU | A | 603 | 32.946 | 102.778 | −20.508 | 1.00 | 38.24 | A |
| ATOM | 4758 | CD1 | LEU | A | 603 | 32.160 | 103.719 | −19.607 | 1.00 | 38.55 | A |
| ATOM | 4759 | CD2 | LEU | A | 603 | 33.775 | 101.811 | −19.677 | 1.00 | 38.91 | A |
| ATOM | 4760 | C | LEU | A | 603 | 29.840 | 100.766 | −21.780 | 1.00 | 34.77 | A |
| ATOM | 4761 | O | LEU | A | 603 | 28.693 | 101.207 | −21.739 | 1.00 | 34.31 | A |
| ATOM | 4762 | N | THR | A | 604 | 30.255 | 99.921 | −22.717 | 1.00 | 32.64 | A |
| ATOM | 4763 | CA | THR | A | 604 | 29.364 | 99.462 | −23.778 | 1.00 | 30.71 | A |
| ATOM | 4764 | CB | THR | A | 604 | 30.162 | 98.985 | −25.002 | 1.00 | 30.78 | A |
| ATOM | 4765 | OG1 | THR | A | 604 | 30.970 | 97.859 | −24.632 | 1.00 | 30.69 | A |
| ATOM | 4766 | CG2 | THR | A | 604 | 31.059 | 100.102 | −25.521 | 1.00 | 30.88 | A |
| ATOM | 4767 | C | THR | A | 604 | 28.459 | 98.321 | −23.329 | 1.00 | 29.37 | A |
| ATOM | 4768 | O | THR | A | 604 | 27.567 | 97.901 | −24.065 | 1.00 | 28.33 | A |
| ATOM | 4769 | N | LYS | A | 605 | 28.693 | 97.821 | −22.121 | 1.00 | 28.18 | A |
| ATOM | 4770 | CA | LYS | A | 605 | 27.897 | 96.723 | −21.582 | 1.00 | 27.68 | A |
| ATOM | 4771 | CB | LYS | A | 605 | 26.457 | 97.185 | −21.341 | 1.00 | 28.11 | A |
| ATOM | 4772 | CG | LYS | A | 605 | 26.335 | 98.324 | −20.344 | 1.00 | 28.53 | A |
| ATOM | 4773 | CD | LYS | A | 605 | 26.807 | 97.904 | −18.959 | 1.00 | 29.48 | A |
| ATOM | 4774 | CE | LYS | A | 605 | 26.707 | 99.058 | −17.972 | 1.00 | 29.77 | A |
| ATOM | 4775 | NZ | LYS | A | 605 | 27.095 | 98.651 | −16.595 | 1.00 | 30.78 | A |
| ATOM | 4776 | C | LYS | A | 605 | 27.898 | 95.510 | −22.511 | 1.00 | 27.31 | A |
| ATOM | 4777 | O | LYS | A | 605 | 26.866 | 94.871 | −22.710 | 1.00 | 26.97 | A |
| ATOM | 4778 | N | THR | A | 606 | 29.058 | 95.206 | −23.085 | 1.00 | 26.32 | A |
| ATOM | 4779 | CA | THR | A | 606 | 29.202 | 94.062 | −23.980 | 1.00 | 25.60 | A |
| ATOM | 4780 | CB | THR | A | 606 | 29.343 | 94.497 | −25.457 | 1.00 | 26.18 | A |
| ATOM | 4781 | OG1 | THR | A | 606 | 30.502 | 95.331 | −25.602 | 1.00 | 26.47 | A |
| ATOM | 4782 | CG2 | THR | A | 606 | 28.109 | 95.259 | −25.912 | 1.00 | 26.21 | A |
| ATOM | 4783 | C | THR | A | 606 | 30.457 | 93.289 | −23.604 | 1.00 | 24.69 | A |
| ATOM | 4784 | O | THR | A | 606 | 31.376 | 93.839 | −23.002 | 1.00 | 24.74 | A |
| ATOM | 4785 | N | ILE | A | 607 | 30.484 | 92.006 | −23.944 | 1.00 | 24.05 | A |
| ATOM | 4786 | CA | ILE | A | 607 | 31.644 | 91.171 | −23.664 | 1.00 | 22.70 | A |
| ATOM | 4787 | CB | ILE | A | 607 | 31.224 | 89.817 | −23.069 | 1.00 | 22.67 | A |
| ATOM | 4788 | CG2 | ILE | A | 607 | 32.460 | 88.983 | −22.758 | 1.00 | 22.01 | A |
| ATOM | 4789 | CG1 | ILE | A | 607 | 30.404 | 90.044 | −21.794 | 1.00 | 22.63 | A |
| ATOM | 4790 | CD1 | ILE | A | 607 | 29.720 | 88.803 | −21.269 | 1.00 | 22.99 | A |
| ATOM | 4791 | C | ILE | A | 607 | 32.333 | 90.954 | −25.007 | 1.00 | 22.58 | A |
| ATOM | 4792 | O | ILE | A | 607 | 31.809 | 90.258 | −25.876 | 1.00 | 21.18 | A |
| ATOM | 4793 | N | HIS | A | 608 | 33.502 | 91.564 | −25.186 | 1.00 | 22.73 | A |
| ATOM | 4794 | CA | HIS | A | 608 | 34.219 | 91.438 | −26.449 | 1.00 | 23.11 | A |
| ATOM | 4795 | CB | HIS | A | 608 | 33.964 | 92.669 | −27.321 | 1.00 | 24.32 | A |
| ATOM | 4796 | CG | HIS | A | 608 | 34.488 | 93.942 | −26.738 | 1.00 | 26.13 | A |
| ATOM | 4797 | CD2 | HIS | A | 608 | 35.450 | 94.791 | −27.172 | 1.00 | 26.91 | A |
| ATOM | 4798 | ND1 | HIS | A | 608 | 34.011 | 94.474 | −25.560 | 1.00 | 27.07 | A |
| ATOM | 4799 | CE1 | HIS | A | 608 | 34.656 | 95.597 | −25.293 | 1.00 | 27.16 | A |
| ATOM | 4800 | NE2 | HIS | A | 608 | 35.535 | 95.811 | −26.256 | 1.00 | 28.09 | A |
| ATOM | 4801 | C | HIS | A | 608 | 35.718 | 91.225 | −26.273 | 1.00 | 22.57 | A |
| ATOM | 4802 | O | HIS | A | 608 | 36.291 | 91.545 | −25.228 | 1.00 | 23.04 | A |
| ATOM | 4803 | N | PRO | A | 609 | 36.378 | 90.684 | −27.308 | 1.00 | 22.03 | A |
| ATOM | 4804 | CD | PRO | A | 609 | 35.781 | 90.110 | −28.530 | 1.00 | 21.85 | A |
| ATOM | 4805 | CA | PRO | A | 609 | 37.814 | 90.419 | −27.261 | 1.00 | 21.59 | A |
| ATOM | 4806 | CB | PRO | A | 609 | 37.950 | 89.193 | −28.147 | 1.00 | 21.45 | A |
| ATOM | 4807 | CG | PRO | A | 609 | 36.992 | 89.524 | −29.258 | 1.00 | 21.74 | A |
| ATOM | 4808 | C | PRO | A | 609 | 38.708 | 91.555 | −27.732 | 1.00 | 21.64 | A |
| ATOM | 4809 | O | PRO | A | 609 | 38.364 | 92.296 | −28.653 | 1.00 | 21.84 | A |
| ATOM | 4810 | N | GLN | A | 610 | 39.859 | 91.678 | −27.082 | 1.00 | 21.61 | A |
| ATOM | 4811 | CA | GLN | A | 610 | 40.851 | 92.682 | −27.434 | 1.00 | 22.66 | A |
| ATOM | 4812 | CB | GLN | A | 610 | 41.132 | 93.622 | −26.256 | 1.00 | 24.57 | A |
| ATOM | 4813 | CG | GLN | A | 610 | 39.950 | 94.484 | −25.830 | 1.00 | 28.22 | A |
| ATOM | 4814 | CD | GLN | A | 610 | 40.335 | 95.514 | −24.778 | 1.00 | 30.69 | A |
| ATOM | 4815 | OE1 | GLN | A | 610 | 40.844 | 95.169 | −23.708 | 1.00 | 32.27 | A |
| ATOM | 4816 | NE2 | GLN | A | 610 | 40.097 | 96.787 | −25.079 | 1.00 | 31.93 | A |
| ATOM | 4817 | C | GLN | A | 610 | 42.115 | 91.908 | −27.782 | 1.00 | 21.64 | A |
| ATOM | 4818 | O | GLN | A | 610 | 42.467 | 90.949 | −27.096 | 1.00 | 21.23 | A |
| ATOM | 4819 | N | GLY | A | 611 | 42.786 | 92.314 | −28.853 | 1.00 | 21.34 | A |
| ATOM | 4820 | CA | GLY | A | 611 | 43.999 | 91.630 | −29.255 | 1.00 | 21.13 | A |
| ATOM | 4821 | C | GLY | A | 611 | 45.250 | 92.379 | −28.846 | 1.00 | 21.53 | A |
| ATOM | 4822 | O | GLY | A | 611 | 45.275 | 93.612 | −28.844 | 1.00 | 20.93 | A |
| ATOM | 4823 | N | SER | A | 612 | 46.292 | 91.635 | −28.489 | 1.00 | 21.82 | A |
| ATOM | 4824 | CA | SER | A | 612 | 47.557 | 92.240 | −28.088 | 1.00 | 22.69 | A |
| ATOM | 4825 | CB | SER | A | 612 | 48.436 | 91.213 | −27.373 | 1.00 | 22.23 | A |
| ATOM | 4826 | OG | SER | A | 612 | 49.741 | 91.726 | −27.170 | 1.00 | 22.93 | A |
| ATOM | 4827 | C | SER | A | 612 | 48.303 | 92.766 | −29.306 | 1.00 | 23.02 | A |
| ATOM | 4828 | O | SER | A | 612 | 48.214 | 92.191 | −30.390 | 1.00 | 22.36 | A |
| ATOM | 4829 | N | THR | A | 613 | 49.042 | 93.859 | −29.124 | 1.00 | 24.55 | A |
| ATOM | 4830 | CA | THR | A | 613 | 49.816 | 94.444 | −30.215 | 1.00 | 26.10 | A |
| ATOM | 4831 | CB | THR | A | 613 | 49.543 | 95.956 | −30.360 | 1.00 | 26.58 | A |
| ATOM | 4832 | OG1 | THR | A | 613 | 49.906 | 96.624 | −29.146 | 1.00 | 26.56 | A |
| ATOM | 4833 | CG2 | THR | A | 613 | 48.069 | 96.209 | −30.659 | 1.00 | 26.70 | A |
| ATOM | 4834 | C | THR | A | 613 | 51.315 | 94.252 | −29.981 | 1.00 | 26.90 | A |
| ATOM | 4835 | O | THR | A | 613 | 52.137 | 94.754 | −30.751 | 1.00 | 27.81 | A |
| ATOM | 4836 | N | THR | A | 614 | 51.664 | 93.516 | −28.926 | 1.00 | 27.11 | A |
| ATOM | 4837 | CA | THR | A | 614 | 53.065 | 93.267 | −28.583 | 1.00 | 26.76 | A |
| ATOM | 4838 | CB | THR | A | 614 | 53.473 | 94.063 | −27.334 | 1.00 | 26.86 | A |
| ATOM | 4839 | OG1 | THR | A | 614 | 52.581 | 93.747 | −26.257 | 1.00 | 27.69 | A |
| ATOM | 4840 | CG2 | THR | A | 614 | 53.423 | 95.559 | −27.611 | 1.00 | 27.62 | A |
| ATOM | 4841 | C | THR | A | 614 | 53.373 | 91.794 | −28.302 | 1.00 | 26.34 | A |
| ATOM | 4842 | O | THR | A | 614 | 54.538 | 91.399 | −28.220 | 1.00 | 26.24 | A |
| ATOM | 4843 | N | LYS | A | 615 | 52.327 | 90.990 | −28.154 | 1.00 | 25.09 | A |
| ATOM | 4844 | CA | LYS | A | 615 | 52.472 | 89.571 | −27.851 | 1.00 | 24.09 | A |
| ATOM | 4845 | CB | LYS | A | 615 | 51.954 | 89.321 | −26.431 | 1.00 | 25.41 | A |
| ATOM | 4846 | CG | LYS | A | 615 | 51.857 | 87.870 | −26.014 | 1.00 | 27.19 | A |
| ATOM | 4847 | CD | LYS | A | 615 | 51.015 | 87.725 | −24.744 | 1.00 | 27.93 | A |
| ATOM | 4848 | CE | LYS | A | 615 | 51.588 | 88.525 | −23.584 | 1.00 | 27.89 | A |
| ATOM | 4849 | NZ | LYS | A | 615 | 50.778 | 88.352 | −22.342 | 1.00 | 27.53 | A |
| ATOM | 4850 | C | LYS | A | 615 | 51.685 | 88.728 | −28.856 | 1.00 | 23.27 | A |
| ATOM | 4851 | O | LYS | A | 615 | 50.591 | 89.115 | −29.270 | 1.00 | 22.28 | A |
| ATOM | 4852 | N | TYR | A | 616 | 52.245 | 87.584 | −29.247 | 1.00 | 21.79 | A |
| ATOM | 4853 | CA | TYR | A | 616 | 51.587 | 86.691 | −30.203 | 1.00 | 21.06 | A |
| ATOM | 4854 | CB | TYR | A | 616 | 52.142 | 86.919 | −31.612 | 1.00 | 21.55 | A |
| ATOM | 4855 | CG | TYR | A | 616 | 52.149 | 88.375 | −32.006 | 1.00 | 22.85 | A |
| ATOM | 4856 | CD1 | TYR | A | 616 | 53.238 | 89.189 | −31.703 | 1.00 | 23.03 | A |
| ATOM | 4857 | CE1 | TYR | A | 616 | 53.219 | 90.549 | −31.980 | 1.00 | 24.34 | A |
| ATOM | 4858 | CD2 | TYR | A | 616 | 51.033 | 88.958 | −32.608 | 1.00 | 23.49 | A |
| ATOM | 4859 | CE2 | TYR | A | 616 | 51.001 | 90.323 | −32.889 | 1.00 | 24.46 | A |
| ATOM | 4860 | CZ | TYR | A | 616 | 52.096 | 91.109 | −32.571 | 1.00 | 24.68 | A |
| ATOM | 4861 | OH | TYR | A | 616 | 52.071 | 92.459 | −32.834 | 1.00 | 26.72 | A |
| ATOM | 4862 | C | TYR | A | 616 | 51.763 | 85.225 | −29.818 | 1.00 | 20.41 | A |
| ATOM | 4863 | O | TYR | A | 616 | 52.678 | 84.882 | −29.074 | 1.00 | 19.70 | A |
| ATOM | 4864 | N | ARG | A | 617 | 50.877 | 84.369 | −30.325 | 1.00 | 19.68 | A |
| ATOM | 4865 | CA | ARG | A | 617 | 50.935 | 82.933 | −30.040 | 1.00 | 19.19 | A |
| ATOM | 4866 | CB | ARG | A | 617 | 49.532 | 82.362 | −29.785 | 1.00 | 19.61 | A |
| ATOM | 4867 | CG | ARG | A | 617 | 48.874 | 82.714 | −28.463 | 1.00 | 20.82 | A |
| ATOM | 4868 | CD | ARG | A | 617 | 47.484 | 82.072 | −28.372 | 1.00 | 20.39 | A |
| ATOM | 4869 | NE | ARG | A | 617 | 47.535 | 80.608 | −28.387 | 1.00 | 20.23 | A |
| ATOM | 4870 | CZ | ARG | A | 617 | 46.866 | 79.838 | −29.242 | 1.00 | 20.36 | A |
| ATOM | 4871 | NH1 | ARG | A | 617 | 46.089 | 80.382 | −30.168 | 1.00 | 20.60 | A |
| ATOM | 4872 | NH2 | ARG | A | 617 | 46.972 | 78.518 | −29.173 | 1.00 | 19.63 | A |
| ATOM | 4873 | C | ARG | A | 617 | 51.528 | 82.154 | −31.208 | 1.00 | 18.48 | A |
| ATOM | 4874 | O | ARG | A | 617 | 51.040 | 82.269 | −32.331 | 1.00 | 19.11 | A |
| ATOM | 4875 | N | ILE | A | 618 | 52.578 | 81.372 | −30.960 | 1.00 | 17.34 | A |
| ATOM | 4876 | CA | ILE | A | 618 | 53.132 | 80.547 | −32.026 | 1.00 | 15.98 | A |
| ATOM | 4877 | CB | ILE | A | 618 | 54.666 | 80.704 | −32.193 | 1.00 | 15.88 | A |
| ATOM | 4878 | CG2 | ILE | A | 618 | 55.399 | 80.260 | −30.938 | 1.00 | 15.95 | A |
| ATOM | 4879 | CG1 | ILE | A | 618 | 55.116 | 79.898 | −33.418 | 1.00 | 16.57 | A |
| ATOM | 4880 | CD1 | ILE | A | 618 | 56.481 | 80.267 | −33.950 | 1.00 | 16.76 | A |
| ATOM | 4881 | C | ILE | A | 618 | 52.771 | 79.113 | −31.666 | 1.00 | 15.58 | A |
| ATOM | 4882 | O | ILE | A | 618 | 52.940 | 78.687 | −30.526 | 1.00 | 15.88 | A |
| ATOM | 4883 | N | ILE | A | 619 | 52.246 | 78.384 | −32.643 | 1.00 | 15.22 | A |
| ATOM | 4884 | CA | ILE | A | 619 | 51.802 | 77.014 | −32.439 | 1.00 | 15.30 | A |
| ATOM | 4885 | CB | ILE | A | 619 | 50.287 | 76.916 | −32.675 | 1.00 | 16.02 | A |
| ATOM | 4886 | CG2 | ILE | A | 619 | 49.788 | 75.529 | −32.309 | 1.00 | 17.07 | A |
| ATOM | 4887 | CG1 | ILE | A | 619 | 49.568 | 78.006 | −31.874 | 1.00 | 16.94 | A |
| ATOM | 4888 | CD1 | ILE | A | 619 | 48.223 | 78.411 | −32.464 | 1.00 | 18.02 | A |
| ATOM | 4889 | C | ILE | A | 619 | 52.484 | 76.047 | −33.399 | 1.00 | 15.21 | A |
| ATOM | 4890 | O | ILE | A | 619 | 52.638 | 76.344 | −34.581 | 1.00 | 14.85 | A |
| ATOM | 4891 | N | PHE | A | 620 | 52.880 | 74.885 | −32.891 | 1.00 | 14.42 | A |
| ATOM | 4892 | CA | PHE | A | 620 | 53.513 | 73.883 | −33.736 | 1.00 | 14.06 | A |
| ATOM | 4893 | CB | PHE | A | 620 | 54.991 | 74.225 | −33.971 | 1.00 | 13.22 | A |
| ATOM | 4894 | CG | PHE | A | 620 | 55.856 | 74.111 | −32.747 | 1.00 | 13.06 | A |
| ATOM | 4895 | CD1 | PHE | A | 620 | 56.459 | 72.898 | −32.416 | 1.00 | 13.53 | A |
| ATOM | 4896 | CD2 | PHE | A | 620 | 56.093 | 75.219 | −31.941 | 1.00 | 12.76 | A |
| ATOM | 4897 | CE1 | PHE | A | 620 | 57.292 | 72.791 | −31.299 | 1.00 | 12.66 | A |
| ATOM | 4898 | CE2 | PHE | A | 620 | 56.925 | 75.125 | −30.822 | 1.00 | 13.48 | A |
| ATOM | 4899 | CZ | PHE | A | 620 | 57.525 | 73.904 | −30.503 | 1.00 | 13.08 | A |
| ATOM | 4900 | C | PHE | A | 620 | 53.366 | 72.492 | −33.137 | 1.00 | 14.55 | A |
| ATOM | 4901 | O | PHE | A | 620 | 53.085 | 72.342 | −31.951 | 1.00 | 14.92 | A |
| ATOM | 4902 | N | LYS | A | 621 | 53.544 | 71.476 | −33.970 | 1.00 | 15.26 | A |
| ATOM | 4903 | CA | LYS | A | 621 | 53.410 | 70.099 | −33.525 | 1.00 | 16.30 | A |
| ATOM | 4904 | CB | LYS | A | 621 | 52.949 | 69.221 | −34.697 | 1.00 | 17.42 | A |
| ATOM | 4905 | CG | LYS | A | 621 | 52.734 | 67.756 | −34.335 | 1.00 | 19.29 | A |
| ATOM | 4906 | CD | LYS | A | 621 | 51.896 | 67.035 | −35.388 | 1.00 | 20.90 | A |
| ATOM | 4907 | CE | LYS | A | 621 | 52.568 | 67.041 | −36.747 | 1.00 | 22.97 | A |
| ATOM | 4908 | NZ | LYS | A | 621 | 51.720 | 66.378 | −37.784 | 1.00 | 25.48 | A |
| ATOM | 4909 | C | LYS | A | 621 | 54.697 | 69.547 | −32.935 | 1.00 | 15.54 | A |
| ATOM | 4910 | O | LYS | A | 621 | 55.717 | 69.466 | −33.617 | 1.00 | 15.67 | A |
| ATOM | 4911 | N | ALA | A | 622 | 54.652 | 69.178 | −31.658 | 1.00 | 15.03 | A |
| ATOM | 4912 | CA | ALA | A | 622 | 55.821 | 68.606 | −31.006 | 1.00 | 15.03 | A |
| ATOM | 4913 | CB | ALA | A | 622 | 55.934 | 69.116 | −29.564 | 1.00 | 14.73 | A |
| ATOM | 4914 | C | ALA | A | 622 | 55.682 | 67.086 | −31.017 | 1.00 | 15.03 | A |
| ATOM | 4915 | O | ALA | A | 622 | 54.585 | 66.554 | −30.851 | 1.00 | 15.48 | A |
| ATOM | 4916 | N | ARG | A | 623 | 56.795 | 66.394 | −31.237 | 1.00 | 15.27 | A |
| ATOM | 4917 | CA | ARG | A | 623 | 56.811 | 64.936 | −31.256 | 1.00 | 14.94 | A |
| ATOM | 4918 | CB | ARG | A | 623 | 57.245 | 64.427 | −32.636 | 1.00 | 16.29 | A |
| ATOM | 4919 | CG | ARG | A | 623 | 57.267 | 62.912 | −32.755 | 1.00 | 18.27 | A |
| ATOM | 4920 | CD | ARG | A | 623 | 57.285 | 62.467 | −34.216 | 1.00 | 20.74 | A |
| ATOM | 4921 | NE | ARG | A | 623 | 57.525 | 61.032 | −34.338 | 1.00 | 22.01 | A |
| ATOM | 4922 | CZ | ARG | A | 623 | 58.723 | 60.467 | −34.232 | 1.00 | 23.12 | A |
| ATOM | 4923 | NH1 | ARG | A | 623 | 59.796 | 61.215 | −34.009 | 1.00 | 24.09 | A |
| ATOM | 4924 | NH2 | ARG | A | 623 | 58.847 | 59.151 | −34.333 | 1.00 | 24.41 | A |
| ATOM | 4925 | C | ARG | A | 623 | 57.804 | 64.530 | −30.176 | 1.00 | 14.36 | A |
| ATOM | 4926 | O | ARG | A | 623 | 58.992 | 64.846 | −30.255 | 1.00 | 13.60 | A |
| ATOM | 4927 | N | VAL | A | 624 | 57.308 | 63.828 | −29.162 | 1.00 | 13.85 | A |
| ATOM | 4928 | CA | VAL | A | 624 | 58.131 | 63.447 | −28.021 | 1.00 | 13.51 | A |
| ATOM | 4929 | CB | VAL | A | 624 | 57.581 | 64.122 | −26.747 | 1.00 | 13.54 | A |
| ATOM | 4930 | CG1 | VAL | A | 624 | 58.635 | 64.120 | −25.645 | 1.00 | 13.13 | A |
| ATOM | 4931 | CG2 | VAL | A | 624 | 57.128 | 65.544 | −27.075 | 1.00 | 14.31 | A |
| ATOM | 4932 | C | VAL | A | 624 | 58.200 | 61.943 | −27.775 | 1.00 | 12.85 | A |
| ATOM | 4933 | O | VAL | A | 624 | 57.204 | 61.243 | −27.906 | 1.00 | 12.59 | A |
| ATOM | 4934 | N | PRO | A | 625 | 59.384 | 61.435 | −27.396 | 1.00 | 13.31 | A |
| ATOM | 4935 | CD | PRO | A | 625 | 60.665 | 62.157 | −27.305 | 1.00 | 13.68 | A |
| ATOM | 4936 | CA | PRO | A | 625 | 59.574 | 60.006 | −27.128 | 1.00 | 12.93 | A |
| ATOM | 4937 | CB | PRO | A | 625 | 61.074 | 59.900 | −26.850 | 1.00 | 13.69 | A |
| ATOM | 4938 | CG | PRO | A | 625 | 61.660 | 61.067 | −27.599 | 1.00 | 14.65 | A |
| ATOM | 4939 | C | PRO | A | 625 | 58.758 | 59.549 | −25.917 | 1.00 | 12.52 | A |
| ATOM | 4940 | O | PRO | A | 625 | 58.333 | 60.365 | −25.099 | 1.00 | 10.81 | A |
| ATOM | 4941 | N | PRO | A | 626 | 58.537 | 58.230 | −25.787 | 1.00 | 12.53 | A |
| ATOM | 4942 | CD | PRO | A | 626 | 59.072 | 57.125 | −26.600 | 1.00 | 11.95 | A |
| ATOM | 4943 | CA | PRO | A | 626 | 57.773 | 57.725 | −24.643 | 1.00 | 12.15 | A |
| ATOM | 4944 | CB | PRO | A | 626 | 57.838 | 56.207 | −24.818 | 1.00 | 12.36 | A |
| ATOM | 4945 | CG | PRO | A | 626 | 58.103 | 56.014 | −26.296 | 1.00 | 12.88 | A |
| ATOM | 4946 | C | PRO | A | 626 | 58.526 | 58.164 | −23.384 | 1.00 | 12.11 | A |
| ATOM | 4947 | O | PRO | A | 626 | 59.747 | 58.026 | −23.325 | 1.00 | 12.43 | A |
| ATOM | 4948 | N | MET | A | 627 | 57.812 | 58.690 | −22.391 | 1.00 | 11.82 | A |
| ATOM | 4949 | CA | MET | A | 627 | 58.444 | 59.126 | −21.146 | 1.00 | 11.98 | A |
| ATOM | 4950 | CB | MET | A | 627 | 58.725 | 57.900 | −20.268 | 1.00 | 12.88 | A |
| ATOM | 4951 | CG | MET | A | 627 | 57.458 | 57.181 | −19.813 | 1.00 | 13.06 | A |
| ATOM | 4952 | SD | MET | A | 627 | 57.753 | 55.522 | −19.152 | 1.00 | 15.42 | A |
| ATOM | 4953 | CE | MET | A | 627 | 58.563 | 55.905 | −17.601 | 1.00 | 15.23 | A |
| ATOM | 4954 | C | MET | A | 627 | 59.745 | 59.877 | −21.434 | 1.00 | 11.93 | A |
| ATOM | 4955 | O | MET | A | 627 | 60.749 | 59.697 | −20.736 | 1.00 | 11.21 | A |
| ATOM | 4956 | N | GLY | A | 628 | 59.716 | 60.737 | −22.453 | 1.00 | 11.85 | A |
| ATOM | 4957 | CA | GLY | A | 628 | 60.917 | 61.460 | −22.830 | 1.00 | 11.48 | A |
| ATOM | 4958 | C | GLY | A | 628 | 60.822 | 62.957 | −23.040 | 1.00 | 11.99 | A |
| ATOM | 4959 | O | GLY | A | 628 | 59.846 | 63.600 | −22.646 | 1.00 | 11.25 | A |
| ATOM | 4960 | N | LEU | A | 629 | 61.854 | 63.506 | −23.678 | 1.00 | 11.91 | A |
| ATOM | 4961 | CA | LEU | A | 629 | 61.943 | 64.938 | −23.941 | 1.00 | 12.42 | A |
| ATOM | 4962 | CB | LEU | A | 629 | 62.964 | 65.569 | −22.996 | 1.00 | 12.70 | A |
| ATOM | 4963 | CG | LEU | A | 629 | 62.725 | 65.362 | −21.500 | 1.00 | 13.60 | A |
| ATOM | 4964 | CD1 | LEU | A | 629 | 63.990 | 65.686 | −20.726 | 1.00 | 14.03 | A |
| ATOM | 4965 | CD2 | LEU | A | 629 | 61.566 | 66.241 | −21.047 | 1.00 | 13.10 | A |
| ATOM | 4966 | C | LEU | A | 629 | 62.372 | 65.222 | −25.378 | 1.00 | 12.55 | A |
| ATOM | 4967 | O | LEU | A | 629 | 63.074 | 64.420 | −25.996 | 1.00 | 12.86 | A |
| ATOM | 4968 | N | ALA | A | 630 | 61.947 | 66.369 | −25.899 | 1.00 | 13.11 | A |
| ATOM | 4969 | CA | ALA | A | 630 | 62.307 | 66.788 | −27.250 | 1.00 | 13.46 | A |
| ATOM | 4970 | CB | ALA | A | 630 | 61.203 | 66.436 | −28.240 | 1.00 | 13.44 | A |
| ATOM | 4971 | C | ALA | A | 630 | 62.534 | 68.293 | −27.218 | 1.00 | 13.75 | A |
| ATOM | 4972 | O | ALA | A | 630 | 61.705 | 69.048 | −26.697 | 1.00 | 13.55 | A |
| ATOM | 4973 | N | THR | A | 631 | 63.660 | 68.721 | −27.779 | 1.00 | 13.65 | A |
| ATOM | 4974 | CA | THR | A | 631 | 64.035 | 70.134 | −27.796 | 1.00 | 13.96 | A |
| ATOM | 4975 | CB | THR | A | 631 | 65.534 | 70.284 | −27.460 | 1.00 | 14.42 | A |
| ATOM | 4976 | OG1 | THR | A | 631 | 65.822 | 69.568 | −26.253 | 1.00 | 13.45 | A |
| ATOM | 4977 | CG2 | THR | A | 631 | 65.910 | 71.751 | −27.280 | 1.00 | 13.92 | A |
| ATOM | 4978 | C | THR | A | 631 | 63.782 | 70.815 | −29.141 | 1.00 | 14.26 | A |
| ATOM | 4979 | O | THR | A | 631 | 64.048 | 70.237 | −30.191 | 1.00 | 14.41 | A |
| ATOM | 4980 | N | TYR | A | 632 | 63.264 | 72.042 | −29.097 | 1.00 | 13.91 | A |
| ATOM | 4981 | CA | TYR | A | 632 | 63.023 | 72.822 | −30.306 | 1.00 | 13.94 | A |
| ATOM | 4982 | CB | TYR | A | 632 | 61.526 | 72.939 | −30.614 | 1.00 | 13.86 | A |
| ATOM | 4983 | CG | TYR | A | 632 | 60.869 | 71.623 | −30.964 | 1.00 | 14.19 | A |
| ATOM | 4984 | CD1 | TYR | A | 632 | 60.486 | 70.726 | −29.967 | 1.00 | 14.07 | A |
| ATOM | 4985 | CE1 | TYR | A | 632 | 59.911 | 69.499 | −30.286 | 1.00 | 14.45 | A |
| ATOM | 4986 | CD2 | TYR | A | 632 | 60.658 | 71.260 | −32.294 | 1.00 | 14.41 | A |
| ATOM | 4987 | CE2 | TYR | A | 632 | 60.083 | 70.032 | −32.626 | 1.00 | 14.34 | A |
| ATOM | 4988 | CZ | TYR | A | 632 | 59.715 | 69.157 | −31.615 | 1.00 | 14.95 | A |
| ATOM | 4989 | OH | TYR | A | 632 | 59.163 | 67.936 | −31.929 | 1.00 | 14.79 | A |
| ATOM | 4990 | C | TYR | A | 632 | 63.618 | 74.214 | −30.122 | 1.00 | 14.02 | A |
| ATOM | 4991 | O | TYR | A | 632 | 63.963 | 74.607 | −29.007 | 1.00 | 13.93 | A |
| ATOM | 4992 | N | VAL | A | 633 | 63.739 | 74.954 | −31.219 | 1.00 | 13.56 | A |
| ATOM | 4993 | CA | VAL | A | 633 | 64.295 | 76.300 | −31.175 | 1.00 | 14.28 | A |
| ATOM | 4994 | CB | VAL | A | 633 | 65.691 | 76.348 | −31.847 | 1.00 | 14.34 | A |
| ATOM | 4995 | CG1 | VAL | A | 633 | 66.245 | 77.778 | −31.817 | 1.00 | 14.97 | A |
| ATOM | 4996 | CG2 | VAL | A | 633 | 66.639 | 75.388 | −31.138 | 1.00 | 15.01 | A |
| ATOM | 4997 | C | VAL | A | 633 | 63.375 | 77.285 | −31.894 | 1.00 | 14.46 | A |
| ATOM | 4998 | O | VAL | A | 633 | 62.889 | 77.005 | −32.990 | 1.00 | 14.06 | A |
| ATOM | 4999 | N | LEU | A | 634 | 63.121 | 78.424 | −31.260 | 1.00 | 14.84 | A |
| ATOM | 5000 | CA | LEU | A | 634 | 62.284 | 79.461 | −31.860 | 1.00 | 15.74 | A |
| ATOM | 5001 | CB | LEU | A | 634 | 61.260 | 79.989 | −30.855 | 1.00 | 16.60 | A |
| ATOM | 5002 | CG | LEU | A | 634 | 60.337 | 78.965 | −30.188 | 1.00 | 18.43 | A |
| ATOM | 5003 | CD1 | LEU | A | 634 | 59.245 | 79.713 | −29.430 | 1.00 | 18.47 | A |
| ATOM | 5004 | CD2 | LEU | A | 634 | 59.723 | 78.042 | −31.224 | 1.00 | 18.25 | A |
| ATOM | 5005 | C | LEU | A | 634 | 63.225 | 80.586 | −32.266 | 1.00 | 16.10 | A |
| ATOM | 5006 | O | LEU | A | 634 | 63.980 | 81.095 | −31.440 | 1.00 | 15.57 | A |
| ATOM | 5007 | N | THR | A | 635 | 63.180 | 80.965 | −33.539 | 1.00 | 16.37 | A |
| ATOM | 5008 | CA | THR | A | 635 | 64.056 | 82.008 | −34.054 | 1.00 | 16.51 | A |
| ATOM | 5009 | CB | THR | A | 635 | 65.024 | 81.425 | −35.095 | 1.00 | 16.04 | A |
| ATOM | 5010 | OG1 | THR | A | 635 | 65.704 | 80.297 | −34.529 | 1.00 | 15.93 | A |
| ATOM | 5011 | CG2 | THR | A | 635 | 66.044 | 82.469 | −35.528 | 1.00 | 15.17 | A |
| ATOM | 5012 | C | THR | A | 635 | 63.260 | 83.132 | −34.708 | 1.00 | 17.49 | A |
| ATOM | 5013 | O | THR | A | 635 | 62.313 | 82.883 | −35.453 | 1.00 | 17.17 | A |
| ATOM | 5014 | N | ILE | A | 636 | 63.656 | 84.370 | −34.434 | 1.00 | 18.82 | A |
| ATOM | 5015 | CA | ILE | A | 636 | 62.970 | 85.524 | −35.003 | 1.00 | 20.25 | A |
| ATOM | 5016 | CB | ILE | A | 636 | 63.010 | 86.730 | −34.028 | 1.00 | 20.80 | A |
| ATOM | 5017 | CG2 | ILE | A | 636 | 64.445 | 87.193 | −33.837 | 1.00 | 20.78 | A |
| ATOM | 5018 | CG1 | ILE | A | 636 | 62.156 | 87.881 | −34.568 | 1.00 | 20.89 | A |
| ATOM | 5019 | CD1 | ILE | A | 636 | 62.027 | 89.054 | −33.608 | 1.00 | 22.07 | A |
| ATOM | 5020 | C | ILE | A | 636 | 63.628 | 85.922 | −36.320 | 1.00 | 21.26 | A |
| ATOM | 5021 | O | ILE | A | 636 | 64.806 | 85.647 | −36.541 | 1.00 | 21.01 | A |
| ATOM | 5022 | N | SER | A | 637 | 62.854 | 86.548 | −37.200 | 1.00 | 23.08 | A |
| ATOM | 5023 | CA | SER | A | 637 | 63.370 | 87.011 | −38.484 | 1.00 | 24.95 | A |
| ATOM | 5024 | CB | SER | A | 637 | 63.007 | 86.033 | −39.608 | 1.00 | 25.80 | A |
| ATOM | 5025 | OG | SER | A | 637 | 61.607 | 85.875 | −39.728 | 1.00 | 28.22 | A |
| ATOM | 5026 | C | SER | A | 637 | 62.765 | 88.386 | −38.755 | 1.00 | 25.93 | A |
| ATOM | 5027 | O | SER | A | 637 | 61.821 | 88.792 | −38.081 | 1.00 | 25.09 | A |
| ATOM | 5028 | N | ASP | A | 638 | 63.310 | 89.105 | −39.729 | 1.00 | 27.88 | A |
| ATOM | 5029 | CA | ASP | A | 638 | 62.805 | 90.437 | −40.042 | 1.00 | 30.30 | A |
| ATOM | 5030 | CB | ASP | A | 638 | 63.804 | 91.193 | −40.928 | 1.00 | 32.47 | A |
| ATOM | 5031 | CG | ASP | A | 638 | 64.112 | 90.463 | −42.221 | 1.00 | 35.12 | A |
| ATOM | 5032 | OD1 | ASP | A | 638 | 63.169 | 90.178 | −42.989 | 1.00 | 36.91 | A |
| ATOM | 5033 | OD2 | ASP | A | 638 | 65.304 | 90.178 | −42.476 | 1.00 | 37.58 | A |
| ATOM | 5034 | C | ASP | A | 638 | 61.438 | 90.398 | −40.717 | 1.00 | 30.50 | A |
| ATOM | 5035 | O | ASP | A | 638 | 60.624 | 91.306 | −40.536 | 1.00 | 31.30 | A |
| ATOM | 5036 | N | SER | A | 639 | 61.181 | 89.339 | −41.479 | 1.00 | 30.26 | A |
| ATOM | 5037 | CA | SER | A | 639 | 59.913 | 89.202 | −42.184 | 1.00 | 30.17 | A |
| ATOM | 5038 | CB | SER | A | 639 | 60.112 | 89.497 | −43.672 | 1.00 | 30.34 | A |
| ATOM | 5039 | OG | SER | A | 639 | 61.072 | 88.619 | −44.232 | 1.00 | 30.45 | A |
| ATOM | 5040 | C | SER | A | 639 | 59.313 | 87.812 | −42.016 | 1.00 | 29.83 | A |
| ATOM | 5041 | O | SER | A | 639 | 59.906 | 86.942 | −41.383 | 1.00 | 29.34 | A |
| ATOM | 5042 | N | LYS | A | 640 | 58.135 | 87.609 | −42.598 | 1.00 | 29.49 | A |
| ATOM | 5043 | CA | LYS | A | 640 | 57.448 | 86.328 | −42.506 | 1.00 | 29.38 | A |
| ATOM | 5044 | CB | LYS | A | 640 | 56.160 | 86.354 | −43.334 | 1.00 | 30.45 | A |
| ATOM | 5045 | CG | LYS | A | 640 | 55.128 | 87.348 | −42.825 | 1.00 | 32.56 | A |
| ATOM | 5046 | CD | LYS | A | 640 | 53.757 | 87.126 | −43.449 | 1.00 | 34.03 | A |
| ATOM | 5047 | CE | LYS | A | 640 | 53.763 | 87.376 | −44.945 | 1.00 | 34.65 | A |
| ATOM | 5048 | NZ | LYS | A | 640 | 52.396 | 87.213 | −45.510 | 1.00 | 35.65 | A |
| ATOM | 5049 | C | LYS | A | 640 | 58.303 | 85.140 | −42.934 | 1.00 | 28.37 | A |
| ATOM | 5050 | O | LYS | A | 640 | 58.718 | 85.041 | −44.088 | 1.00 | 28.39 | A |
| ATOM | 5051 | N | PRO | A | 641 | 58.581 | 84.221 | −41.996 | 1.00 | 27.43 | A |
| ATOM | 5052 | CD | PRO | A | 641 | 58.213 | 84.267 | −40.571 | 1.00 | 27.24 | A |
| ATOM | 5053 | CA | PRO | A | 641 | 59.387 | 83.034 | −42.288 | 1.00 | 26.57 | A |
| ATOM | 5054 | CB | PRO | A | 641 | 59.674 | 82.465 | −40.901 | 1.00 | 27.13 | A |
| ATOM | 5055 | CG | PRO | A | 641 | 58.461 | 82.844 | −40.132 | 1.00 | 27.56 | A |
| ATOM | 5056 | C | PRO | A | 641 | 58.630 | 82.056 | −43.187 | 1.00 | 25.67 | A |
| ATOM | 5057 | O | PRO | A | 641 | 57.400 | 82.001 | −43.176 | 1.00 | 24.50 | A |
| ATOM | 5058 | N | GLU | A | 642 | 59.382 | 81.282 | −43.958 | 1.00 | 25.02 | A |
| ATOM | 5059 | CA | GLU | A | 642 | 58.813 | 80.318 | −44.891 | 1.00 | 24.72 | A |
| ATOM | 5060 | CB | GLU | A | 642 | 59.945 | 79.622 | −45.658 | 1.00 | 26.15 | A |
| ATOM | 5061 | CG | GLU | A | 642 | 59.484 | 78.486 | −46.562 | 1.00 | 27.49 | A |
| ATOM | 5062 | CD | GLU | A | 642 | 60.635 | 77.795 | −47.278 | 1.00 | 29.77 | A |
| ATOM | 5063 | OE1 | GLU | A | 642 | 60.378 | 76.791 | −47.977 | 1.00 | 30.73 | A |
| ATOM | 5064 | OE2 | GLU | A | 642 | 61.791 | 78.252 | −47.144 | 1.00 | 30.00 | A |
| ATOM | 5065 | C | GLU | A | 642 | 57.885 | 79.255 | −44.303 | 1.00 | 24.21 | A |
| ATOM | 5066 | O | GLU | A | 642 | 56.865 | 78.921 | −44.903 | 1.00 | 24.08 | A |
| ATOM | 5067 | N | HIS | A | 643 | 58.223 | 78.728 | −43.133 | 1.00 | 23.20 | A |
| ATOM | 5068 | CA | HIS | A | 643 | 57.416 | 77.665 | −42.538 | 1.00 | 22.09 | A |
| ATOM | 5069 | CB | HIS | A | 643 | 58.345 | 76.582 | −41.992 | 1.00 | 23.02 | A |
| ATOM | 5070 | CG | HIS | A | 643 | 59.256 | 76.004 | −43.029 | 1.00 | 24.08 | A |
| ATOM | 5071 | CD2 | HIS | A | 643 | 60.556 | 76.255 | −43.312 | 1.00 | 24.46 | A |
| ATOM | 5072 | ND1 | HIS | A | 643 | 58.831 | 75.083 | −43.961 | 1.00 | 25.14 | A |
| ATOM | 5073 | CE1 | HIS | A | 643 | 59.830 | 74.789 | −44.774 | 1.00 | 25.08 | A |
| ATOM | 5074 | NE2 | HIS | A | 643 | 60.888 | 75.487 | −44.402 | 1.00 | 24.83 | A |
| ATOM | 5075 | C | HIS | A | 643 | 56.420 | 78.078 | −41.464 | 1.00 | 21.26 | A |
| ATOM | 5076 | O | HIS | A | 643 | 55.944 | 77.238 | −40.697 | 1.00 | 20.11 | A |
| ATOM | 5077 | N | THR | A | 644 | 56.101 | 79.365 | −41.411 | 1.00 | 20.08 | A |
| ATOM | 5078 | CA | THR | A | 644 | 55.146 | 79.865 | −40.435 | 1.00 | 20.00 | A |
| ATOM | 5079 | CB | THR | A | 644 | 55.805 | 80.877 | −39.475 | 1.00 | 20.19 | A |
| ATOM | 5080 | OG1 | THR | A | 644 | 56.886 | 80.236 | −38.782 | 1.00 | 19.60 | A |
| ATOM | 5081 | CG2 | THR | A | 644 | 54.791 | 81.395 | −38.451 | 1.00 | 19.41 | A |
| ATOM | 5082 | C | THR | A | 644 | 53.986 | 80.542 | −41.162 | 1.00 | 20.34 | A |
| ATOM | 5083 | O | THR | A | 644 | 54.200 | 81.413 | −42.009 | 1.00 | 20.31 | A |
| ATOM | 5084 | N | SER | A | 645 | 52.765 | 80.121 | −40.847 | 1.00 | 19.47 | A |
| ATOM | 5085 | CA | SER | A | 645 | 51.576 | 80.702 | −41.459 | 1.00 | 18.89 | A |
| ATOM | 5086 | CB | SER | A | 645 | 50.636 | 79.604 | −41.977 | 1.00 | 19.65 | A |
| ATOM | 5087 | OG | SER | A | 645 | 50.103 | 78.821 | −40.917 | 1.00 | 18.59 | A |
| ATOM | 5088 | C | SER | A | 645 | 50.859 | 81.556 | −40.420 | 1.00 | 18.42 | A |
| ATOM | 5089 | O | SER | A | 645 | 51.124 | 81.445 | −39.217 | 1.00 | 17.35 | A |
| ATOM | 5090 | N | TYR | A | 646 | 49.951 | 82.408 | −40.882 | 1.00 | 17.29 | A |
| ATOM | 5091 | CA | TYR | A | 646 | 49.211 | 83.278 | −39.979 | 1.00 | 16.80 | A |
| ATOM | 5092 | CB | TYR | A | 646 | 49.590 | 84.737 | −40.243 | 1.00 | 17.06 | A |
| ATOM | 5093 | CG | TYR | A | 646 | 51.069 | 84.965 | −40.045 | 1.00 | 17.26 | A |
| ATOM | 5094 | CD1 | TYR | A | 646 | 51.980 | 84.670 | −41.059 | 1.00 | 17.65 | A |
| ATOM | 5095 | CE1 | TYR | A | 646 | 53.352 | 84.771 | −40.843 | 1.00 | 17.99 | A |
| ATOM | 5096 | CD2 | TYR | A | 646 | 51.567 | 85.374 | −38.811 | 1.00 | 17.57 | A |
| ATOM | 5097 | CE2 | TYR | A | 646 | 52.935 | 85.478 | −38.584 | 1.00 | 17.55 | A |
| ATOM | 5098 | CZ | TYR | A | 646 | 53.820 | 85.173 | −39.602 | 1.00 | 17.60 | A |
| ATOM | 5099 | OH | TYR | A | 646 | 55.174 | 85.253 | −39.373 | 1.00 | 18.18 | A |
| ATOM | 5100 | C | TYR | A | 646 | 47.712 | 83.081 | −40.097 | 1.00 | 16.23 | A |
| ATOM | 5101 | O | TYR | A | 646 | 47.158 | 83.071 | −41.194 | 1.00 | 16.02 | A |
| ATOM | 5102 | N | ALA | A | 647 | 47.062 | 82.916 | −38.951 | 1.00 | 15.52 | A |
| ATOM | 5103 | CA | ALA | A | 647 | 45.627 | 82.699 | −38.906 | 1.00 | 15.34 | A |
| ATOM | 5104 | CB | ALA | A | 647 | 45.219 | 82.251 | −37.507 | 1.00 | 16.44 | A |
| ATOM | 5105 | C | ALA | A | 647 | 44.837 | 83.939 | −39.290 | 1.00 | 15.45 | A |
| ATOM | 5106 | O | ALA | A | 647 | 45.273 | 85.066 | −39.072 | 1.00 | 15.26 | A |
| ATOM | 5107 | N | SER | A | 648 | 43.669 | 83.722 | −39.877 | 1.00 | 16.15 | A |
| ATOM | 5108 | CA | SER | A | 648 | 42.802 | 84.829 | −40.232 | 1.00 | 16.33 | A |
| ATOM | 5109 | CB | SER | A | 648 | 41.952 | 84.479 | −41.456 | 1.00 | 17.26 | A |
| ATOM | 5110 | OG | SER | A | 648 | 41.126 | 83.360 | −41.194 | 1.00 | 18.74 | A |
| ATOM | 5111 | C | SER | A | 648 | 41.914 | 84.985 | −39.005 | 1.00 | 16.21 | A |
| ATOM | 5112 | O | SER | A | 648 | 41.754 | 84.037 | −38.230 | 1.00 | 16.15 | A |
| ATOM | 5113 | N | ASN | A | 649 | 41.348 | 86.171 | −38.815 | 1.00 | 15.76 | A |
| ATOM | 5114 | CA | ASN | A | 649 | 40.481 | 86.413 | −37.669 | 1.00 | 15.87 | A |
| ATOM | 5115 | CB | ASN | A | 649 | 41.216 | 87.235 | −36.605 | 1.00 | 15.87 | A |
| ATOM | 5116 | CG | ASN | A | 649 | 42.396 | 86.491 | −36.000 | 1.00 | 15.98 | A |
| ATOM | 5117 | OD1 | ASN | A | 649 | 42.250 | 85.747 | −35.027 | 1.00 | 15.07 | A |
| ATOM | 5118 | ND2 | ASN | A | 649 | 43.573 | 86.684 | −36.583 | 1.00 | 15.26 | A |
| ATOM | 5119 | C | ASN | A | 649 | 39.236 | 87.156 | −38.126 | 1.00 | 16.17 | A |
| ATOM | 5120 | O | ASN | A | 649 | 39.321 | 88.131 | −38.877 | 1.00 | 15.97 | A |
| ATOM | 5121 | N | LEU | A | 650 | 38.083 | 86.686 | −37.666 | 1.00 | 16.93 | A |
| ATOM | 5122 | CA | LEU | A | 650 | 36.804 | 87.288 | −38.020 | 1.00 | 17.30 | A |
| ATOM | 5123 | CB | LEU | A | 650 | 36.025 | 86.353 | −38.946 | 1.00 | 16.88 | A |
| ATOM | 5124 | CG | LEU | A | 650 | 34.612 | 86.785 | −39.340 | 1.00 | 17.13 | A |
| ATOM | 5125 | CD1 | LEU | A | 650 | 34.670 | 88.040 | −40.202 | 1.00 | 17.16 | A |
| ATOM | 5126 | CD2 | LEU | A | 650 | 33.933 | 85.649 | −40.091 | 1.00 | 16.83 | A |
| ATOM | 5127 | C | LEU | A | 650 | 35.985 | 87.567 | −36.768 | 1.00 | 18.02 | A |
| ATOM | 5128 | O | LEU | A | 650 | 35.627 | 86.648 | −36.025 | 1.00 | 17.29 | A |
| ATOM | 5129 | N | LEU | A | 651 | 35.687 | 88.842 | −36.540 | 1.00 | 18.59 | A |
| ATOM | 5130 | CA | LEU | A | 651 | 34.911 | 89.255 | −35.380 | 1.00 | 19.22 | A |
| ATOM | 5131 | CB | LEU | A | 651 | 35.512 | 90.534 | −34.783 | 1.00 | 20.86 | A |
| ATOM | 5132 | CG | LEU | A | 651 | 35.142 | 90.917 | −33.345 | 1.00 | 21.95 | A |
| ATOM | 5133 | CD1 | LEU | A | 651 | 33.696 | 91.353 | −33.262 | 1.00 | 23.51 | A |
| ATOM | 5134 | CD2 | LEU | A | 651 | 35.403 | 89.734 | −32.429 | 1.00 | 22.19 | A |
| ATOM | 5135 | C | LEU | A | 651 | 33.473 | 89.500 | −35.825 | 1.00 | 19.82 | A |
| ATOM | 5136 | O | LEU | A | 651 | 33.191 | 90.449 | −36.558 | 1.00 | 19.72 | A |
| ATOM | 5137 | N | LEU | A | 652 | 32.565 | 88.639 | −35.378 | 1.00 | 19.69 | A |
| ATOM | 5138 | CA | LEU | A | 652 | 31.166 | 88.757 | −35.755 | 1.00 | 19.98 | A |
| ATOM | 5139 | CB | LEU | A | 652 | 30.586 | 87.375 | −36.052 | 1.00 | 19.30 | A |
| ATOM | 5140 | CG | LEU | A | 652 | 31.315 | 86.605 | −37.156 | 1.00 | 19.06 | A |
| ATOM | 5141 | CD1 | LEU | A | 652 | 30.723 | 85.206 | −37.283 | 1.00 | 18.94 | A |
| ATOM | 5142 | CD2 | LEU | A | 652 | 31.207 | 87.368 | −38.477 | 1.00 | 18.77 | A |
| ATOM | 5143 | C | LEU | A | 652 | 30.320 | 89.449 | −34.700 | 1.00 | 21.51 | A |
| ATOM | 5144 | O | LEU | A | 652 | 30.212 | 88.988 | −33.560 | 1.00 | 20.61 | A |
| ATOM | 5145 | N | ARG | A | 653 | 29.726 | 90.570 | −35.094 | 1.00 | 22.71 | A |
| ATOM | 5146 | CA | ARG | A | 653 | 28.865 | 91.332 | −34.207 | 1.00 | 24.81 | A |
| ATOM | 5147 | CB | ARG | A | 653 | 29.582 | 91.658 | −32.901 | 1.00 | 25.37 | A |
| ATOM | 5148 | CG | ARG | A | 653 | 30.646 | 92.727 | −33.008 | 1.00 | 27.04 | A |
| ATOM | 5149 | CD | ARG | A | 653 | 30.498 | 93.677 | −31.839 | 1.00 | 29.21 | A |
| ATOM | 5150 | NE | ARG | A | 653 | 31.775 | 94.064 | −31.261 | 1.00 | 30.48 | A |
| ATOM | 5151 | CZ | ARG | A | 653 | 31.897 | 94.765 | −30.141 | 1.00 | 31.74 | A |
| ATOM | 5152 | NH1 | ARG | A | 653 | 30.814 | 95.156 | −29.482 | 1.00 | 32.11 | A |
| ATOM | 5153 | NH2 | ARG | A | 653 | 33.100 | 95.072 | −29.678 | 1.00 | 32.44 | A |
| ATOM | 5154 | C | ARG | A | 653 | 28.435 | 92.626 | −34.868 | 1.00 | 25.66 | A |
| ATOM | 5155 | O | ARG | A | 653 | 29.062 | 93.087 | −35.821 | 1.00 | 24.91 | A |
| ATOM | 5156 | N | LYS | A | 654 | 27.360 | 93.207 | −34.354 | 1.00 | 27.38 | A |
| ATOM | 5157 | CA | LYS | A | 654 | 26.858 | 94.463 | −34.879 | 1.00 | 29.48 | A |
| ATOM | 5158 | CB | LYS | A | 654 | 25.366 | 94.605 | −34.568 | 1.00 | 30.53 | A |
| ATOM | 5159 | CG | LYS | A | 654 | 24.503 | 93.495 | −35.159 | 1.00 | 32.00 | A |
| ATOM | 5160 | CD | LYS | A | 654 | 23.402 | 94.053 | −36.050 | 1.00 | 33.77 | A |
| ATOM | 5161 | CE | LYS | A | 654 | 23.983 | 94.797 | −37.244 | 1.00 | 34.37 | A |
| ATOM | 5162 | NZ | LYS | A | 654 | 22.917 | 95.372 | −38.117 | 1.00 | 36.17 | A |
| ATOM | 5163 | C | LYS | A | 654 | 27.645 | 95.580 | −34.204 | 1.00 | 29.80 | A |
| ATOM | 5164 | O | LYS | A | 654 | 28.156 | 95.405 | −33.097 | 1.00 | 30.41 | A |
| ATOM | 5165 | N | ASN | A | 655 | 27.751 | 96.718 | −34.878 | 1.00 | 30.31 | A |
| ATOM | 5166 | CA | ASN | A | 655 | 28.470 | 97.865 | −34.337 | 1.00 | 30.30 | A |
| ATOM | 5167 | CB | ASN | A | 655 | 27.714 | 98.427 | −33.134 | 1.00 | 32.65 | A |
| ATOM | 5168 | CG | ASN | A | 655 | 26.223 | 98.526 | −33.384 | 1.00 | 34.87 | A |
| ATOM | 5169 | OD1 | ASN | A | 655 | 25.784 | 99.111 | −34.379 | 1.00 | 36.57 | A |
| ATOM | 5170 | ND2 | ASN | A | 655 | 25.432 | 97.951 | −32.482 | 1.00 | 35.70 | A |
| ATOM | 5171 | C | ASN | A | 655 | 29.892 | 97.499 | −33.920 | 1.00 | 29.09 | A |
| ATOM | 5172 | O | ASN | A | 655 | 30.272 | 97.657 | −32.760 | 1.00 | 28.95 | A |
| ATOM | 5173 | N | PRO | A | 656 | 30.699 | 97.000 | −34.867 | 1.00 | 28.12 | A |
| ATOM | 5174 | CD | PRO | A | 656 | 30.370 | 96.630 | −36.257 | 1.00 | 27.68 | A |
| ATOM | 5175 | CA | PRO | A | 656 | 32.076 | 96.627 | −34.544 | 1.00 | 27.59 | A |
| ATOM | 5176 | CB | PRO | A | 656 | 32.417 | 95.634 | −35.643 | 1.00 | 27.64 | A |
| ATOM | 5177 | CG | PRO | A | 656 | 31.728 | 96.247 | −36.827 | 1.00 | 27.46 | A |
| ATOM | 5178 | C | PRO | A | 656 | 33.008 | 97.832 | −34.570 | 1.00 | 27.27 | A |
| ATOM | 5179 | O | PRO | A | 656 | 32.700 | 98.856 | −35.180 | 1.00 | 26.04 | A |
| ATOM | 5180 | N | THR | A | 657 | 34.143 | 97.700 | −33.893 | 1.00 | 26.93 | A |
| ATOM | 5181 | CA | THR | A | 657 | 35.152 | 98.748 | −33.864 | 1.00 | 27.12 | A |
| ATOM | 5182 | CB | THR | A | 657 | 35.302 | 99.365 | −32.455 | 1.00 | 27.52 | A |
| ATOM | 5183 | OG1 | THR | A | 657 | 35.484 | 98.324 | −31.489 | 1.00 | 27.21 | A |
| ATOM | 5184 | CG2 | THR | A | 657 | 34.067 | 100.186 | −32.099 | 1.00 | 27.48 | A |
| ATOM | 5185 | C | THR | A | 657 | 36.462 | 98.090 | −34.280 | 1.00 | 27.11 | A |
| ATOM | 5186 | O | THR | A | 657 | 36.618 | 96.873 | −34.158 | 1.00 | 27.18 | A |
| ATOM | 5187 | N | SER | A | 658 | 37.395 | 98.890 | −34.779 | 1.00 | 26.83 | A |
| ATOM | 5188 | CA | SER | A | 658 | 38.682 | 98.380 | −35.229 | 1.00 | 26.73 | A |
| ATOM | 5189 | CB | SER | A | 658 | 39.576 | 99.539 | −35.671 | 1.00 | 26.98 | A |
| ATOM | 5190 | OG | SER | A | 658 | 39.848 | 100.401 | −34.581 | 1.00 | 27.98 | A |
| ATOM | 5191 | C | SER | A | 658 | 39.401 | 97.559 | −34.159 | 1.00 | 26.39 | A |
| ATOM | 5192 | O | SER | A | 658 | 39.191 | 97.753 | −32.961 | 1.00 | 25.68 | A |
| ATOM | 5193 | N | LEU | A | 659 | 40.246 | 96.639 | −34.616 | 1.00 | 25.99 | A |
| ATOM | 5194 | CA | LEU | A | 659 | 41.023 | 95.772 | −33.739 | 1.00 | 25.88 | A |
| ATOM | 5195 | CB | LEU | A | 659 | 40.381 | 94.383 | −33.649 | 1.00 | 26.05 | A |
| ATOM | 5196 | CG | LEU | A | 659 | 39.071 | 94.250 | −32.868 | 1.00 | 25.86 | A |
| ATOM | 5197 | CD1 | LEU | A | 659 | 38.453 | 92.874 | −33.107 | 1.00 | 26.79 | A |
| ATOM | 5198 | CD2 | LEU | A | 659 | 39.342 | 94.466 | −31.392 | 1.00 | 25.65 | A |
| ATOM | 5199 | C | LEU | A | 659 | 42.437 | 95.640 | −34.294 | 1.00 | 25.93 | A |
| ATOM | 5200 | O | LEU | A | 659 | 42.758 | 94.663 | −34.974 | 1.00 | 26.37 | A |
| ATOM | 5201 | N | PRO | A | 660 | 43.297 | 96.635 | −34.030 | 1.00 | 25.74 | A |
| ATOM | 5202 | CD | PRO | A | 660 | 43.052 | 97.866 | −33.256 | 1.00 | 25.96 | A |
| ATOM | 5203 | CA | PRO | A | 660 | 44.679 | 96.592 | −34.520 | 1.00 | 25.77 | A |
| ATOM | 5204 | CB | PRO | A | 660 | 45.195 | 97.994 | −34.209 | 1.00 | 25.89 | A |
| ATOM | 5205 | CG | PRO | A | 660 | 44.463 | 98.340 | −32.952 | 1.00 | 25.60 | A |
| ATOM | 5206 | C | PRO | A | 660 | 45.461 | 95.497 | −33.793 | 1.00 | 25.59 | A |
| ATOM | 5207 | O | PRO | A | 660 | 45.154 | 95.169 | −32.649 | 1.00 | 25.45 | A |
| ATOM | 5208 | N | LEU | A | 661 | 46.469 | 94.938 | −34.453 | 1.00 | 26.17 | A |
| ATOM | 5209 | CA | LEU | A | 661 | 47.256 | 93.867 | −33.846 | 1.00 | 26.47 | A |
| ATOM | 5210 | CB | LEU | A | 661 | 46.827 | 92.522 | −34.439 | 1.00 | 26.01 | A |
| ATOM | 5211 | CG | LEU | A | 661 | 45.373 | 92.111 | −34.197 | 1.00 | 25.85 | A |
| ATOM | 5212 | CD1 | LEU | A | 661 | 45.053 | 90.850 | −34.990 | 1.00 | 25.80 | A |
| ATOM | 5213 | CD2 | LEU | A | 661 | 45.148 | 91.885 | −32.707 | 1.00 | 25.90 | A |
| ATOM | 5214 | C | LEU | A | 661 | 48.764 | 94.046 | −34.013 | 1.00 | 26.80 | A |
| ATOM | 5215 | O | LEU | A | 661 | 49.495 | 93.076 | −34.217 | 1.00 | 26.97 | A |
| ATOM | 5216 | N | GLY | A | 662 | 49.228 | 95.287 | −33.925 | 1.00 | 27.05 | A |
| ATOM | 5217 | CA | GLY | A | 662 | 50.649 | 95.549 | −34.066 | 1.00 | 27.15 | A |
| ATOM | 5218 | C | GLY | A | 662 | 51.246 | 95.016 | −35.356 | 1.00 | 27.55 | A |
| ATOM | 5219 | O | GLY | A | 662 | 50.791 | 95.355 | −36.447 | 1.00 | 27.88 | A |
| ATOM | 5220 | N | GLN | A | 663 | 52.260 | 94.165 | −35.228 | 1.00 | 27.70 | A |
| ATOM | 5221 | CA | GLN | A | 663 | 52.948 | 93.594 | −36.383 | 1.00 | 27.75 | A |
| ATOM | 5222 | CB | GLN | A | 663 | 54.338 | 93.111 | −35.972 | 1.00 | 29.23 | A |
| ATOM | 5223 | CG | GLN | A | 663 | 55.162 | 94.123 | −35.205 | 1.00 | 31.06 | A |
| ATOM | 5224 | CD | GLN | A | 663 | 56.475 | 93.535 | −34.733 | 1.00 | 31.96 | A |
| ATOM | 5225 | OE1 | GLN | A | 663 | 57.324 | 93.155 | −35.544 | 1.00 | 32.29 | A |
| ATOM | 5226 | NE2 | GLN | A | 663 | 56.647 | 93.445 | −33.418 | 1.00 | 31.97 | A |
| ATOM | 5227 | C | GLN | A | 663 | 52.227 | 92.434 | −37.063 | 1.00 | 26.71 | A |
| ATOM | 5228 | O | GLN | A | 663 | 52.634 | 92.003 | −38.140 | 1.00 | 26.75 | A |
| ATOM | 5229 | N | TYR | A | 664 | 51.168 | 91.923 | −36.445 | 1.00 | 26.28 | A |
| ATOM | 5230 | CA | TYR | A | 664 | 50.438 | 90.796 | −37.020 | 1.00 | 25.87 | A |
| ATOM | 5231 | CB | TYR | A | 664 | 49.138 | 90.557 | −36.251 | 1.00 | 24.54 | A |
| ATOM | 5232 | CG | TYR | A | 664 | 48.551 | 89.181 | −36.475 | 1.00 | 22.88 | A |
| ATOM | 5233 | CD1 | TYR | A | 664 | 49.145 | 88.047 | −35.915 | 1.00 | 22.04 | A |
| ATOM | 5234 | CE1 | TYR | A | 664 | 48.608 | 86.775 | −36.124 | 1.00 | 21.08 | A |
| ATOM | 5235 | CD2 | TYR | A | 664 | 47.407 | 89.010 | −37.254 | 1.00 | 21.95 | A |
| ATOM | 5236 | CE2 | TYR | A | 664 | 46.866 | 87.748 | −37.472 | 1.00 | 20.70 | A |
| ATOM | 5237 | CZ | TYR | A | 664 | 47.469 | 86.634 | −36.904 | 1.00 | 21.25 | A |
| ATOM | 5238 | OH | TYR | A | 664 | 46.923 | 85.387 | −37.114 | 1.00 | 19.20 | A |
| ATOM | 5239 | C | TYR | A | 664 | 50.146 | 91.023 | −38.507 | 1.00 | 26.39 | A |
| ATOM | 5240 | O | TYR | A | 664 | 49.503 | 92.002 | −38.884 | 1.00 | 26.43 | A |
| ATOM | 5241 | N | PRO | A | 665 | 50.618 | 90.104 | −39.367 | 1.00 | 27.21 | A |
| ATOM | 5242 | CD | PRO | A | 665 | 51.335 | 88.902 | −38.908 | 1.00 | 26.98 | A |
| ATOM | 5243 | CA | PRO | A | 665 | 50.490 | 90.087 | −40.830 | 1.00 | 27.74 | A |
| ATOM | 5244 | CB | PRO | A | 665 | 51.064 | 88.722 | −41.209 | 1.00 | 27.97 | A |
| ATOM | 5245 | CG | PRO | A | 665 | 52.074 | 88.481 | −40.153 | 1.00 | 27.69 | A |
| ATOM | 5246 | C | PRO | A | 665 | 49.111 | 90.302 | −41.455 | 1.00 | 28.39 | A |
| ATOM | 5247 | O | PRO | A | 665 | 48.996 | 90.999 | −42.463 | 1.00 | 28.61 | A |
| ATOM | 5248 | N | GLU | A | 666 | 48.071 | 89.703 | −40.881 | 1.00 | 28.52 | A |
| ATOM | 5249 | CA | GLU | A | 666 | 46.738 | 89.842 | −41.461 | 1.00 | 28.27 | A |
| ATOM | 5250 | CB | GLU | A | 666 | 46.116 | 88.464 | −41.709 | 1.00 | 30.04 | A |
| ATOM | 5251 | CG | GLU | A | 666 | 44.880 | 88.533 | −42.593 | 1.00 | 33.21 | A |
| ATOM | 5252 | CD | GLU | A | 666 | 44.407 | 87.178 | −43.071 | 1.00 | 34.36 | A |
| ATOM | 5253 | OE1 | GLU | A | 666 | 45.226 | 86.434 | −43.652 | 1.00 | 36.02 | A |
| ATOM | 5254 | OE2 | GLU | A | 666 | 43.214 | 86.863 | −42.876 | 1.00 | 34.72 | A |
| ATOM | 5255 | C | GLU | A | 666 | 45.770 | 90.691 | −40.651 | 1.00 | 27.30 | A |
| ATOM | 5256 | O | GLU | A | 666 | 45.712 | 90.595 | −39.425 | 1.00 | 26.86 | A |
| ATOM | 5257 | N | ASP | A | 667 | 45.003 | 91.515 | −41.361 | 1.00 | 25.51 | A |
| ATOM | 5258 | CA | ASP | A | 667 | 44.024 | 92.403 | −40.741 | 1.00 | 24.38 | A |
| ATOM | 5259 | CB | ASP | A | 667 | 43.629 | 93.518 | −41.715 | 1.00 | 25.91 | A |
| ATOM | 5260 | CG | ASP | A | 667 | 44.821 | 94.302 | −42.220 | 1.00 | 27.61 | A |
| ATOM | 5261 | OD1 | ASP | A | 667 | 45.584 | 94.826 | −41.382 | 1.00 | 28.24 | A |
| ATOM | 5262 | OD2 | ASP | A | 667 | 44.993 | 94.393 | −43.454 | 1.00 | 28.96 | A |
| ATOM | 5263 | C | ASP | A | 667 | 42.765 | 91.654 | −40.324 | 1.00 | 22.12 | A |
| ATOM | 5264 | O | ASP | A | 667 | 42.275 | 90.789 | −41.047 | 1.00 | 21.74 | A |
| ATOM | 5265 | N | VAL | A | 668 | 42.239 | 91.999 | −39.159 | 1.00 | 20.90 | A |
| ATOM | 5266 | CA | VAL | A | 668 | 41.025 | 91.372 | −38.662 | 1.00 | 20.03 | A |
| ATOM | 5267 | CB | VAL | A | 668 | 40.715 | 91.827 | −37.221 | 1.00 | 19.83 | A |
| ATOM | 5268 | CG1 | VAL | A | 668 | 39.392 | 91.228 | −36.754 | 1.00 | 19.61 | A |
| ATOM | 5269 | CG2 | VAL | A | 668 | 41.851 | 91.408 | −36.291 | 1.00 | 19.72 | A |
| ATOM | 5270 | C | VAL | A | 668 | 39.863 | 91.769 | −39.565 | 1.00 | 20.16 | A |
| ATOM | 5271 | O | VAL | A | 668 | 39.808 | 92.901 | −40.053 | 1.00 | 19.96 | A |
| ATOM | 5272 | N | LYS | A | 669 | 38.948 | 90.831 | −39.792 | 1.00 | 19.66 | A |
| ATOM | 5273 | CA | LYS | A | 669 | 37.770 | 91.067 | −40.623 | 1.00 | 20.28 | A |
| ATOM | 5274 | CB | LYS | A | 669 | 37.615 | 89.935 | −41.641 | 1.00 | 21.78 | A |
| ATOM | 5275 | CG | LYS | A | 669 | 38.806 | 89.805 | −42.586 | 1.00 | 25.50 | A |
| ATOM | 5276 | CD | LYS | A | 669 | 38.919 | 88.405 | −43.182 | 1.00 | 28.10 | A |
| ATOM | 5277 | CE | LYS | A | 669 | 40.207 | 88.254 | −43.992 | 1.00 | 29.47 | A |
| ATOM | 5278 | NZ | LYS | A | 669 | 40.464 | 86.846 | −44.414 | 1.00 | 29.99 | A |
| ATOM | 5279 | C | LYS | A | 669 | 36.547 | 91.131 | −39.713 | 1.00 | 19.68 | A |
| ATOM | 5280 | O | LYS | A | 669 | 36.546 | 90.538 | −38.631 | 1.00 | 18.99 | A |
| ATOM | 5281 | N | PHE | A | 670 | 35.513 | 91.851 | −40.142 | 1.00 | 18.45 | A |
| ATOM | 5282 | CA | PHE | A | 670 | 34.303 | 91.984 | −39.341 | 1.00 | 18.50 | A |
| ATOM | 5283 | CB | PHE | A | 670 | 34.158 | 93.421 | −38.824 | 1.00 | 18.47 | A |
| ATOM | 5284 | CG | PHE | A | 670 | 35.344 | 93.907 | −38.045 | 1.00 | 18.53 | A |
| ATOM | 5285 | CD1 | PHE | A | 670 | 36.476 | 94.382 | −38.701 | 1.00 | 18.06 | A |
| ATOM | 5286 | CD2 | PHE | A | 670 | 35.345 | 93.859 | −36.655 | 1.00 | 18.27 | A |
| ATOM | 5287 | CE1 | PHE | A | 670 | 37.593 | 94.800 | −37.981 | 1.00 | 18.42 | A |
| ATOM | 5288 | CE2 | PHE | A | 670 | 36.457 | 94.275 | −35.928 | 1.00 | 19.20 | A |
| ATOM | 5289 | CZ | PHE | A | 670 | 37.583 | 94.745 | −36.593 | 1.00 | 18.35 | A |
| ATOM | 5290 | C | PHE | A | 670 | 33.049 | 91.600 | −40.121 | 1.00 | 18.27 | A |
| ATOM | 5291 | O | PHE | A | 670 | 33.090 | 91.430 | −41.339 | 1.00 | 18.35 | A |
| ATOM | 5292 | N | GLY | A | 671 | 31.937 | 91.460 | −39.410 | 1.00 | 18.50 | A |
| ATOM | 5293 | CA | GLY | A | 671 | 30.687 | 91.106 | −40.059 | 1.00 | 18.15 | A |
| ATOM | 5294 | C | GLY | A | 671 | 29.565 | 90.910 | −39.062 | 1.00 | 18.46 | A |
| ATOM | 5295 | O | GLY | A | 671 | 29.804 | 90.822 | −37.858 | 1.00 | 17.12 | A |
| ATOM | 5296 | N | ASP | A | 672 | 28.332 | 90.853 | −39.553 | 1.00 | 18.63 | A |
| ATOM | 5297 | CA | ASP | A | 672 | 27.196 | 90.639 | −38.671 | 1.00 | 19.58 | A |
| ATOM | 5298 | CB | ASP | A | 672 | 25.873 | 90.951 | −39.376 | 1.00 | 21.20 | A |
| ATOM | 5299 | CG | ASP | A | 672 | 25.692 | 92.426 | −39.673 | 1.00 | 22.59 | A |
| ATOM | 5300 | OD1 | ASP | A | 672 | 26.358 | 93.258 | −39.024 | 1.00 | 24.13 | A |
| ATOM | 5301 | OD2 | ASP | A | 672 | 24.861 | 92.751 | −40.547 | 1.00 | 23.78 | A |
| ATOM | 5302 | C | ASP | A | 672 | 27.189 | 89.173 | −38.263 | 1.00 | 19.65 | A |
| ATOM | 5303 | O | ASP | A | 672 | 27.695 | 88.318 | −38.987 | 1.00 | 18.43 | A |
| ATOM | 5304 | N | PRO | A | 673 | 26.618 | 88.865 | −37.091 | 1.00 | 19.89 | A |
| ATOM | 5305 | CD | PRO | A | 673 | 26.012 | 89.784 | −36.111 | 1.00 | 20.15 | A |
| ATOM | 5306 | CA | PRO | A | 673 | 26.557 | 87.480 | −36.619 | 1.00 | 20.31 | A |
| ATOM | 5307 | CB | PRO | A | 673 | 25.659 | 87.580 | −35.392 | 1.00 | 20.38 | A |
| ATOM | 5308 | CG | PRO | A | 673 | 25.978 | 88.940 | −34.859 | 1.00 | 20.50 | A |
| ATOM | 5309 | C | PRO | A | 673 | 25.950 | 86.592 | −37.705 | 1.00 | 20.50 | A |
| ATOM | 5310 | O | PRO | A | 673 | 25.009 | 86.992 | −38.392 | 1.00 | 19.86 | A |
| ATOM | 5311 | N | ARG | A | 674 | 26.499 | 85.396 | −37.870 | 1.00 | 20.93 | A |
| ATOM | 5312 | CA | ARG | A | 674 | 25.997 | 84.463 | −38.871 | 1.00 | 21.72 | A |
| ATOM | 5313 | CB | ARG | A | 674 | 26.445 | 84.881 | −40.276 | 1.00 | 21.99 | A |
| ATOM | 5314 | CG | ARG | A | 674 | 27.941 | 84.754 | −40.537 | 1.00 | 22.77 | A |
| ATOM | 5315 | CD | ARG | A | 674 | 28.221 | 84.804 | −42.033 | 1.00 | 24.81 | A |
| ATOM | 5316 | NE | ARG | A | 674 | 29.603 | 84.475 | −42.369 | 1.00 | 26.31 | A |
| ATOM | 5317 | CZ | ARG | A | 674 | 30.609 | 85.343 | −42.367 | 1.00 | 27.19 | A |
| ATOM | 5318 | NH1 | ARG | A | 674 | 30.399 | 86.614 | −42.046 | 1.00 | 28.25 | A |
| ATOM | 5319 | NH2 | ARG | A | 674 | 31.828 | 84.939 | −42.701 | 1.00 | 27.77 | A |
| ATOM | 5320 | C | ARG | A | 674 | 26.516 | 83.065 | −38.575 | 1.00 | 21.85 | A |
| ATOM | 5321 | O | ARG | A | 674 | 27.503 | 82.903 | −37.859 | 1.00 | 21.35 | A |
| ATOM | 5322 | N | GLU | A | 675 | 25.850 | 82.052 | −39.120 | 1.00 | 22.23 | A |
| ATOM | 5323 | CA | GLU | A | 675 | 26.294 | 80.688 | −38.897 | 1.00 | 22.85 | A |
| ATOM | 5324 | CB | GLU | A | 675 | 25.243 | 79.685 | −39.378 | 1.00 | 24.28 | A |
| ATOM | 5325 | CG | GLU | A | 675 | 23.886 | 79.863 | −38.724 | 1.00 | 25.01 | A |
| ATOM | 5326 | CD | GLU | A | 675 | 23.065 | 78.591 | −38.738 | 1.00 | 27.02 | A |
| ATOM | 5327 | OE1 | GLU | A | 675 | 23.076 | 77.884 | −39.769 | 1.00 | 26.65 | A |
| ATOM | 5328 | OE2 | GLU | A | 675 | 22.403 | 78.305 | −37.716 | 1.00 | 27.89 | A |
| ATOM | 5329 | C | GLU | A | 675 | 27.593 | 80.480 | −39.655 | 1.00 | 23.14 | A |
| ATOM | 5330 | O | GLU | A | 675 | 27.812 | 81.081 | −40.710 | 1.00 | 23.14 | A |
| ATOM | 5331 | N | ILE | A | 676 | 28.467 | 79.644 | −39.106 | 1.00 | 22.54 | A |
| ATOM | 5332 | CA | ILE | A | 676 | 29.740 | 79.375 | −39.752 | 1.00 | 22.87 | A |
| ATOM | 5333 | CB | ILE | A | 676 | 30.892 | 80.167 | −39.105 | 1.00 | 24.28 | A |
| ATOM | 5334 | CG2 | ILE | A | 676 | 30.575 | 81.657 | −39.112 | 1.00 | 25.64 | A |
| ATOM | 5335 | CG1 | ILE | A | 676 | 31.117 | 79.680 | −37.677 | 1.00 | 24.43 | A |
| ATOM | 5336 | CD1 | ILE | A | 676 | 32.453 | 80.090 | −37.106 | 1.00 | 26.48 | A |
| ATOM | 5337 | C | ILE | A | 676 | 30.086 | 77.901 | −39.664 | 1.00 | 22.36 | A |
| ATOM | 5338 | O | ILE | A | 676 | 29.569 | 77.174 | −38.814 | 1.00 | 22.01 | A |
| ATOM | 5339 | N | SER | A | 677 | 30.971 | 77.476 | −40.555 | 1.00 | 21.98 | A |
| ATOM | 5340 | CA | SER | A | 677 | 31.428 | 76.099 | −40.606 | 1.00 | 22.60 | A |
| ATOM | 5341 | CB | SER | A | 677 | 30.861 | 75.404 | −41.845 | 1.00 | 22.88 | A |
| ATOM | 5342 | OG | SER | A | 677 | 31.340 | 74.077 | −41.939 | 1.00 | 25.66 | A |
| ATOM | 5343 | C | SER | A | 677 | 32.949 | 76.128 | −40.673 | 1.00 | 22.27 | A |
| ATOM | 5344 | O | SER | A | 677 | 33.528 | 77.004 | −41.313 | 1.00 | 22.32 | A |
| ATOM | 5345 | N | LEU | A | 678 | 33.594 | 75.178 | −40.004 | 1.00 | 21.75 | A |
| ATOM | 5346 | CA | LEU | A | 678 | 35.050 | 75.113 | −39.997 | 1.00 | 21.96 | A |
| ATOM | 5347 | CB | LEU | A | 678 | 35.613 | 75.741 | −38.718 | 1.00 | 22.31 | A |
| ATOM | 5348 | CG | LEU | A | 678 | 35.622 | 77.264 | −38.562 | 1.00 | 22.76 | A |
| ATOM | 5349 | CD1 | LEU | A | 678 | 36.046 | 77.627 | −37.146 | 1.00 | 22.99 | A |
| ATOM | 5350 | CD2 | LEU | A | 678 | 36.579 | 77.875 | −39.567 | 1.00 | 23.14 | A |
| ATOM | 5351 | C | LEU | A | 678 | 35.551 | 73.681 | −40.091 | 1.00 | 22.13 | A |
| ATOM | 5352 | O | LEU | A | 678 | 34.892 | 72.748 | −39.624 | 1.00 | 21.03 | A |
| ATOM | 5353 | N | ARG | A | 679 | 36.723 | 73.524 | −40.695 | 1.00 | 22.18 | A |
| ATOM | 5354 | CA | ARG | A | 679 | 37.347 | 72.218 | −40.834 | 1.00 | 23.61 | A |
| ATOM | 5355 | CB | ARG | A | 679 | 36.904 | 71.530 | −42.127 | 1.00 | 24.89 | A |
| ATOM | 5356 | CG | ARG | A | 679 | 37.397 | 70.095 | −42.210 | 1.00 | 27.40 | A |
| ATOM | 5357 | CD | ARG | A | 679 | 37.123 | 69.453 | −43.555 | 1.00 | 29.86 | A |
| ATOM | 5358 | NE | ARG | A | 679 | 37.544 | 68.055 | −43.546 | 1.00 | 32.30 | A |
| ATOM | 5359 | CZ | ARG | A | 679 | 37.557 | 67.266 | −44.615 | 1.00 | 33.42 | A |
| ATOM | 5360 | NH1 | ARG | A | 679 | 37.172 | 67.736 | −45.795 | 1.00 | 34.22 | A |
| ATOM | 5361 | NH2 | ARG | A | 679 | 37.958 | 66.007 | −44.502 | 1.00 | 34.11 | A |
| ATOM | 5362 | C | ARG | A | 679 | 38.866 | 72.346 | −40.837 | 1.00 | 23.38 | A |
| ATOM | 5363 | O | ARG | A | 679 | 39.440 | 73.049 | −41.672 | 1.00 | 23.17 | A |
| ATOM | 5364 | N | VAL | A | 680 | 39.514 | 71.672 | −39.893 | 1.00 | 22.77 | A |
| ATOM | 5365 | CA | VAL | A | 680 | 40.967 | 71.692 | −39.809 | 1.00 | 22.34 | A |
| ATOM | 5366 | CB | VAL | A | 680 | 41.439 | 71.950 | −38.366 | 1.00 | 22.26 | A |
| ATOM | 5367 | CG1 | VAL | A | 680 | 42.954 | 71.866 | −38.292 | 1.00 | 21.52 | A |
| ATOM | 5368 | CG2 | VAL | A | 680 | 40.960 | 73.325 | −37.906 | 1.00 | 21.17 | A |
| ATOM | 5369 | C | VAL | A | 680 | 41.500 | 70.342 | −40.281 | 1.00 | 23.12 | A |
| ATOM | 5370 | O | VAL | A | 680 | 41.005 | 69.293 | −39.866 | 1.00 | 22.47 | A |
| ATOM | 5371 | N | GLY | A | 681 | 42.505 | 70.377 | −41.152 | 1.00 | 24.09 | A |
| ATOM | 5372 | CA | GLY | A | 681 | 43.078 | 69.149 | −41.673 | 1.00 | 25.82 | A |
| ATOM | 5373 | C | GLY | A | 681 | 42.009 | 68.272 | −42.298 | 1.00 | 27.00 | A |
| ATOM | 5374 | O | GLY | A | 681 | 41.084 | 68.773 | −42.939 | 1.00 | 26.66 | A |
| ATOM | 5375 | N | ASN | A | 682 | 42.130 | 66.962 | −42.113 | 1.00 | 28.40 | A |
| ATOM | 5376 | CA | ASN | A | 682 | 41.152 | 66.024 | −42.654 | 1.00 | 30.08 | A |
| ATOM | 5377 | CB | ASN | A | 682 | 41.848 | 64.786 | −43.220 | 1.00 | 32.01 | A |
| ATOM | 5378 | CG | ASN | A | 682 | 42.620 | 65.080 | −44.491 | 1.00 | 34.27 | A |
| ATOM | 5379 | OD1 | ASN | A | 682 | 43.168 | 64.173 | −45.116 | 1.00 | 36.87 | A |
| ATOM | 5380 | ND2 | ASN | A | 682 | 42.666 | 66.349 | −44.882 | 1.00 | 35.56 | A |
| ATOM | 5381 | C | ASN | A | 682 | 40.186 | 65.605 | −41.556 | 1.00 | 29.67 | A |
| ATOM | 5382 | O | ASN | A | 682 | 39.448 | 64.632 | −41.702 | 1.00 | 30.68 | A |
| ATOM | 5383 | N | GLY | A | 683 | 40.199 | 66.353 | −40.456 | 1.00 | 28.63 | A |
| ATOM | 5384 | CA | GLY | A | 683 | 39.329 | 66.050 | −39.336 | 1.00 | 27.13 | A |
| ATOM | 5385 | C | GLY | A | 683 | 37.867 | 66.320 | −39.622 | 1.00 | 25.71 | A |
| ATOM | 5386 | O | GLY | A | 683 | 37.476 | 66.450 | −40.782 | 1.00 | 25.68 | A |
| ATOM | 5387 | N | PRO | A | 684 | 37.027 | 66.410 | −38.578 | 1.00 | 24.04 | A |
| ATOM | 5388 | CD | PRO | A | 684 | 37.354 | 66.244 | −37.150 | 1.00 | 24.02 | A |
| ATOM | 5389 | CA | PRO | A | 684 | 35.597 | 66.667 | −38.759 | 1.00 | 23.26 | A |
| ATOM | 5390 | CB | PRO | A | 684 | 35.014 | 66.321 | −37.394 | 1.00 | 23.35 | A |
| ATOM | 5391 | CG | PRO | A | 684 | 36.104 | 66.763 | −36.462 | 1.00 | 24.03 | A |
| ATOM | 5392 | C | PRO | A | 684 | 35.298 | 68.108 | −39.159 | 1.00 | 22.03 | A |
| ATOM | 5393 | O | PRO | A | 684 | 36.136 | 68.996 | −39.004 | 1.00 | 21.92 | A |
| ATOM | 5394 | N | THR | A | 685 | 34.103 | 68.322 | −39.694 | 1.00 | 21.27 | A |
| ATOM | 5395 | CA | THR | A | 685 | 33.661 | 69.653 | −40.091 | 1.00 | 20.07 | A |
| ATOM | 5396 | CB | THR | A | 685 | 33.047 | 69.649 | −41.506 | 1.00 | 19.84 | A |
| ATOM | 5397 | OG1 | THR | A | 685 | 34.051 | 69.284 | −42.458 | 1.00 | 19.96 | A |
| ATOM | 5398 | CG2 | THR | A | 685 | 32.495 | 71.029 | −41.855 | 1.00 | 19.80 | A |
| ATOM | 5399 | C | THR | A | 685 | 32.596 | 70.041 | −39.078 | 1.00 | 19.41 | A |
| ATOM | 5400 | O | THR | A | 685 | 31.600 | 69.336 | −38.918 | 1.00 | 19.79 | A |
| ATOM | 5401 | N | LEU | A | 686 | 32.811 | 71.156 | −38.388 | 1.00 | 18.82 | A |
| ATOM | 5402 | CA | LEU | A | 686 | 31.872 | 71.612 | −37.375 | 1.00 | 18.60 | A |
| ATOM | 5403 | CB | LEU | A | 686 | 32.606 | 71.908 | −36.061 | 1.00 | 18.99 | A |
| ATOM | 5404 | CG | LEU | A | 686 | 33.539 | 70.849 | −35.466 | 1.00 | 20.30 | A |
| ATOM | 5405 | CD1 | LEU | A | 686 | 33.969 | 71.304 | −34.072 | 1.00 | 19.83 | A |
| ATOM | 5406 | CD2 | LEU | A | 686 | 32.849 | 69.497 | −35.393 | 1.00 | 20.32 | A |
| ATOM | 5407 | C | LEU | A | 686 | 31.109 | 72.856 | −37.807 | 1.00 | 18.14 | A |
| ATOM | 5408 | O | LEU | A | 686 | 31.679 | 73.775 | −38.397 | 1.00 | 17.46 | A |
| ATOM | 5409 | N | ALA | A | 687 | 29.815 | 72.871 | −37.505 | 1.00 | 17.55 | A |
| ATOM | 5410 | CA | ALA | A | 687 | 28.956 | 73.998 | −37.841 | 1.00 | 18.33 | A |
| ATOM | 5411 | CB | ALA | A | 687 | 27.759 | 73.526 | −38.658 | 1.00 | 18.09 | A |
| ATOM | 5412 | C | ALA | A | 687 | 28.483 | 74.653 | −36.552 | 1.00 | 18.18 | A |
| ATOM | 5413 | O | ALA | A | 687 | 28.116 | 73.966 | −35.596 | 1.00 | 18.07 | A |
| ATOM | 5414 | N | PHE | A | 688 | 28.488 | 75.981 | −36.536 | 1.00 | 17.57 | A |
| ATOM | 5415 | CA | PHE | A | 688 | 28.073 | 76.741 | −35.365 | 1.00 | 16.78 | A |
| ATOM | 5416 | CB | PHE | A | 688 | 29.242 | 77.575 | −34.830 | 1.00 | 16.30 | A |
| ATOM | 5417 | CG | PHE | A | 688 | 30.477 | 76.778 | −34.526 | 1.00 | 15.14 | A |
| ATOM | 5418 | CD1 | PHE | A | 688 | 31.286 | 76.298 | −35.552 | 1.00 | 14.69 | A |
| ATOM | 5419 | CD2 | PHE | A | 688 | 30.837 | 76.513 | −33.208 | 1.00 | 15.04 | A |
| ATOM | 5420 | CE1 | PHE | A | 688 | 32.437 | 75.567 | −35.271 | 1.00 | 14.91 | A |
| ATOM | 5421 | CE2 | PHE | A | 688 | 31.986 | 75.783 | −32.915 | 1.00 | 14.60 | A |
| ATOM | 5422 | CZ | PHE | A | 688 | 32.788 | 75.309 | −33.948 | 1.00 | 14.65 | A |
| ATOM | 5423 | C | PHE | A | 688 | 26.921 | 77.683 | −35.689 | 1.00 | 17.40 | A |
| ATOM | 5424 | O | PHE | A | 688 | 26.760 | 78.119 | −36.830 | 1.00 | 18.04 | A |
| ATOM | 5425 | N | SER | A | 689 | 26.129 | 78.002 | −34.674 | 1.00 | 17.46 | A |
| ATOM | 5426 | CA | SER | A | 689 | 25.006 | 78.916 | −34.837 | 1.00 | 18.27 | A |
| ATOM | 5427 | CB | SER | A | 689 | 24.012 | 78.745 | −33.689 | 1.00 | 17.90 | A |
| ATOM | 5428 | OG | SER | A | 689 | 24.564 | 79.219 | −32.473 | 1.00 | 17.73 | A |
| ATOM | 5429 | C | SER | A | 689 | 25.574 | 80.334 | −34.815 | 1.00 | 18.53 | A |
| ATOM | 5430 | O | SER | A | 689 | 26.765 | 80.525 | −34.561 | 1.00 | 17.93 | A |
| ATOM | 5431 | N | GLU | A | 690 | 24.729 | 81.326 | −35.077 | 1.00 | 19.26 | A |
| ATOM | 5432 | CA | GLU | A | 690 | 25.188 | 82.709 | −35.077 | 1.00 | 20.14 | A |
| ATOM | 5433 | CB | GLU | A | 690 | 24.130 | 83.629 | −35.697 | 1.00 | 22.21 | A |
| ATOM | 5434 | CG | GLU | A | 690 | 22.856 | 83.773 | −34.890 | 1.00 | 24.08 | A |
| ATOM | 5435 | CD | GLU | A | 690 | 21.919 | 84.806 | −35.489 | 1.00 | 26.32 | A |
| ATOM | 5436 | OE1 | GLU | A | 690 | 21.439 | 84.588 | −36.623 | 1.00 | 26.83 | A |
| ATOM | 5437 | OE2 | GLU | A | 690 | 21.670 | 85.838 | −34.829 | 1.00 | 28.62 | A |
| ATOM | 5438 | C | GLU | A | 690 | 25.539 | 83.188 | −33.669 | 1.00 | 19.89 | A |
| ATOM | 5439 | O | GLU | A | 690 | 26.028 | 84.304 | −33.490 | 1.00 | 19.45 | A |
| ATOM | 5440 | N | GLN | A | 691 | 25.279 | 82.347 | −32.672 | 1.00 | 19.85 | A |
| ATOM | 5441 | CA | GLN | A | 691 | 25.611 | 82.677 | −31.289 | 1.00 | 20.01 | A |
| ATOM | 5442 | CB | GLN | A | 691 | 24.519 | 82.182 | −30.340 | 1.00 | 22.21 | A |
| ATOM | 5443 | CG | GLN | A | 691 | 23.204 | 82.921 | −30.479 | 1.00 | 25.23 | A |
| ATOM | 5444 | CD | GLN | A | 691 | 22.014 | 81.998 | −30.363 | 1.00 | 27.55 | A |
| ATOM | 5445 | OE1 | GLN | A | 691 | 21.813 | 81.345 | −29.336 | 1.00 | 28.65 | A |
| ATOM | 5446 | NE2 | GLN | A | 691 | 21.215 | 81.934 | −31.423 | 1.00 | 29.19 | A |
| ATOM | 5447 | C | GLN | A | 691 | 26.948 | 82.025 | −30.925 | 1.00 | 19.08 | A |
| ATOM | 5448 | O | GLN | A | 691 | 27.359 | 82.030 | −29.766 | 1.00 | 19.03 | A |
| ATOM | 5449 | N | GLY | A | 692 | 27.615 | 81.457 | −31.925 | 1.00 | 18.05 | A |
| ATOM | 5450 | CA | GLY | A | 692 | 28.907 | 80.828 | −31.702 | 1.00 | 17.80 | A |
| ATOM | 5451 | C | GLY | A | 692 | 28.877 | 79.489 | −30.986 | 1.00 | 17.67 | A |
| ATOM | 5452 | O | GLY | A | 692 | 29.878 | 79.067 | −30.409 | 1.00 | 18.07 | A |
| ATOM | 5453 | N | LEU | A | 693 | 27.735 | 78.813 | −31.024 | 1.00 | 17.63 | A |
| ATOM | 5454 | CA | LEU | A | 693 | 27.601 | 77.515 | −30.368 | 1.00 | 17.63 | A |
| ATOM | 5455 | CB | LEU | A | 693 | 26.369 | 77.518 | −29.462 | 1.00 | 19.17 | A |
| ATOM | 5456 | CG | LEU | A | 693 | 26.470 | 78.433 | −28.239 | 1.00 | 20.12 | A |
| ATOM | 5457 | CD1 | LEU | A | 693 | 25.076 | 78.780 | −27.735 | 1.00 | 22.01 | A |
| ATOM | 5458 | CD2 | LEU | A | 693 | 27.290 | 77.755 | −27.158 | 1.00 | 20.87 | A |
| ATOM | 5459 | C | LEU | A | 693 | 27.503 | 76.373 | −31.367 | 1.00 | 17.45 | A |
| ATOM | 5460 | O | LEU | A | 693 | 26.901 | 76.511 | −32.435 | 1.00 | 16.57 | A |
| ATOM | 5461 | N | LEU | A | 694 | 28.095 | 75.238 | −31.008 | 1.00 | 17.12 | A |
| ATOM | 5462 | CA | LEU | A | 694 | 28.085 | 74.060 | −31.866 | 1.00 | 17.42 | A |
| ATOM | 5463 | CB | LEU | A | 694 | 28.785 | 72.893 | −31.164 | 1.00 | 17.16 | A |
| ATOM | 5464 | CG | LEU | A | 694 | 28.960 | 71.618 | −31.993 | 1.00 | 16.87 | A |
| ATOM | 5465 | CD1 | LEU | A | 694 | 29.935 | 71.876 | −33.132 | 1.00 | 16.72 | A |
| ATOM | 5466 | CD2 | LEU | A | 694 | 29.480 | 70.495 | −31.097 | 1.00 | 16.91 | A |
| ATOM | 5467 | C | LEU | A | 694 | 26.658 | 73.659 | −32.223 | 1.00 | 17.52 | A |
| ATOM | 5468 | O | LEU | A | 694 | 25.764 | 73.707 | −31.380 | 1.00 | 16.45 | A |
| ATOM | 5469 | N | LYS | A | 695 | 26.458 | 73.264 | −33.478 | 1.00 | 18.82 | A |
| ATOM | 5470 | CA | LYS | A | 695 | 25.147 | 72.847 | −33.961 | 1.00 | 20.55 | A |
| ATOM | 5471 | CB | LYS | A | 695 | 24.614 | 73.868 | −34.971 | 1.00 | 22.63 | A |
| ATOM | 5472 | CG | LYS | A | 695 | 23.347 | 73.447 | −35.697 | 1.00 | 26.75 | A |
| ATOM | 5473 | CD | LYS | A | 695 | 22.670 | 74.641 | −36.378 | 1.00 | 29.24 | A |
| ATOM | 5474 | CE | LYS | A | 695 | 22.138 | 75.631 | −35.345 | 1.00 | 30.84 | A |
| ATOM | 5475 | NZ | LYS | A | 695 | 21.443 | 76.797 | −35.961 | 1.00 | 32.11 | A |
| ATOM | 5476 | C | LYS | A | 695 | 25.197 | 71.466 | −34.602 | 1.00 | 20.01 | A |
| ATOM | 5477 | O | LYS | A | 695 | 24.230 | 70.710 | −34.527 | 1.00 | 20.35 | A |
| ATOM | 5478 | N | SER | A | 696 | 26.323 | 71.136 | −35.230 | 1.00 | 19.98 | A |
| ATOM | 5479 | CA | SER | A | 696 | 26.470 | 69.838 | −35.882 | 1.00 | 19.65 | A |
| ATOM | 5480 | CB | SER | A | 696 | 25.755 | 69.844 | −37.238 | 1.00 | 19.79 | A |
| ATOM | 5481 | OG | SER | A | 696 | 26.472 | 70.618 | −38.188 | 1.00 | 19.06 | A |
| ATOM | 5482 | C | SER | A | 696 | 27.926 | 69.450 | −36.095 | 1.00 | 19.73 | A |
| ATOM | 5483 | O | SER | A | 696 | 28.813 | 70.308 | −36.131 | 1.00 | 19.51 | A |
| ATOM | 5484 | N | ILE | A | 697 | 28.157 | 68.148 | −36.243 | 1.00 | 18.94 | A |
| ATOM | 5485 | CA | ILE | A | 697 | 29.488 | 67.599 | −36.476 | 1.00 | 19.29 | A |
| ATOM | 5486 | CB | ILE | A | 697 | 30.004 | 66.795 | −35.257 | 1.00 | 18.22 | A |
| ATOM | 5487 | CG2 | ILE | A | 697 | 31.349 | 66.159 | −35.584 | 1.00 | 17.95 | A |
| ATOM | 5488 | CG1 | ILE | A | 697 | 30.130 | 67.701 | −34.032 | 1.00 | 17.15 | A |
| ATOM | 5489 | CD1 | ILE | A | 697 | 30.598 | 66.968 | −32.782 | 1.00 | 16.28 | A |
| ATOM | 5490 | C | ILE | A | 697 | 29.435 | 66.633 | −37.660 | 1.00 | 20.96 | A |
| ATOM | 5491 | O | ILE | A | 697 | 28.591 | 65.734 | −37.694 | 1.00 | 21.08 | A |
| ATOM | 5492 | N | GLN | A | 698 | 30.325 | 66.825 | −38.627 | 1.00 | 22.27 | A |
| ATOM | 5493 | CA | GLN | A | 698 | 30.396 | 65.943 | −39.786 | 1.00 | 23.77 | A |
| ATOM | 5494 | CB | GLN | A | 698 | 30.292 | 66.730 | −41.096 | 1.00 | 24.36 | A |
| ATOM | 5495 | CG | GLN | A | 698 | 29.972 | 65.833 | −42.291 | 1.00 | 25.87 | A |
| ATOM | 5496 | CD | GLN | A | 698 | 30.216 | 66.504 | −43.631 | 1.00 | 27.18 | A |
| ATOM | 5497 | OE1 | GLN | A | 698 | 29.671 | 66.083 | −44.654 | 1.00 | 28.35 | A |
| ATOM | 5498 | NE2 | GLN | A | 698 | 31.046 | 67.538 | −43.635 | 1.00 | 27.17 | A |
| ATOM | 5499 | C | GLN | A | 698 | 31.743 | 65.235 | −39.725 | 1.00 | 24.33 | A |
| ATOM | 5500 | O | GLN | A | 698 | 32.788 | 65.850 | −39.951 | 1.00 | 24.42 | A |
| ATOM | 5501 | N | LEU | A | 699 | 31.719 | 63.940 | −39.423 | 1.00 | 25.41 | A |
| ATOM | 5502 | CA | LEU | A | 699 | 32.944 | 63.160 | −39.301 | 1.00 | 26.65 | A |
| ATOM | 5503 | CB | LEU | A | 699 | 32.615 | 61.743 | −38.821 | 1.00 | 26.18 | A |
| ATOM | 5504 | CG | LEU | A | 699 | 31.898 | 61.659 | −37.467 | 1.00 | 25.56 | A |
| ATOM | 5505 | CD1 | LEU | A | 699 | 31.704 | 60.203 | −37.076 | 1.00 | 24.95 | A |
| ATOM | 5506 | CD2 | LEU | A | 699 | 32.713 | 62.387 | −36.410 | 1.00 | 25.37 | A |
| ATOM | 5507 | C | LEU | A | 699 | 33.785 | 63.095 | −40.569 | 1.00 | 28.22 | A |
| ATOM | 5508 | O | LEU | A | 699 | 35.008 | 63.219 | −40.514 | 1.00 | 28.15 | A |
| ATOM | 5509 | N | THR | A | 700 | 33.137 | 62.895 | −41.711 | 1.00 | 30.08 | A |
| ATOM | 5510 | CA | THR | A | 700 | 33.853 | 62.820 | −42.979 | 1.00 | 32.39 | A |
| ATOM | 5511 | CB | THR | A | 700 | 33.986 | 61.360 | −43.462 | 1.00 | 32.28 | A |
| ATOM | 5512 | OG1 | THR | A | 700 | 32.682 | 60.798 | −43.660 | 1.00 | 32.31 | A |
| ATOM | 5513 | CG2 | THR | A | 700 | 34.740 | 60.531 | −42.438 | 1.00 | 31.92 | A |
| ATOM | 5514 | C | THR | A | 700 | 33.123 | 63.625 | −44.045 | 1.00 | 33.91 | A |
| ATOM | 5515 | O | THR | A | 700 | 31.955 | 63.968 | −43.878 | 1.00 | 33.85 | A |
| ATOM | 5516 | N | GLN | A | 701 | 33.816 | 63.927 | −45.137 | 1.00 | 36.39 | A |
| ATOM | 5517 | CA | GLN | A | 701 | 33.222 | 64.698 | −46.224 | 1.00 | 38.80 | A |
| ATOM | 5518 | CB | GLN | A | 701 | 34.245 | 64.908 | −47.342 | 1.00 | 39.86 | A |
| ATOM | 5519 | CG | GLN | A | 701 | 35.569 | 65.462 | −46.860 | 1.00 | 41.67 | A |
| ATOM | 5520 | CD | GLN | A | 701 | 36.454 | 65.926 | −47.998 | 1.00 | 42.85 | A |
| ATOM | 5521 | OE1 | GLN | A | 701 | 36.088 | 66.830 | −48.753 | 1.00 | 43.40 | A |
| ATOM | 5522 | NE2 | GLN | A | 701 | 37.628 | 65.313 | −48.129 | 1.00 | 43.29 | A |
| ATOM | 5523 | C | GLN | A | 701 | 31.997 | 63.987 | −46.782 | 1.00 | 39.49 | A |
| ATOM | 5524 | O | GLN | A | 701 | 31.102 | 64.617 | −47.343 | 1.00 | 40.50 | A |
| ATOM | 5525 | N | ASP | A | 702 | 31.962 | 62.669 | −46.615 | 1.00 | 40.44 | A |
| ATOM | 5526 | CA | ASP | A | 702 | 30.854 | 61.856 | −47.101 | 1.00 | 41.22 | A |
| ATOM | 5527 | CB | ASP | A | 702 | 31.325 | 60.416 | −47.314 | 1.00 | 42.70 | A |
| ATOM | 5528 | CG | ASP | A | 702 | 32.717 | 60.341 | −47.909 | 1.00 | 43.88 | A |
| ATOM | 5529 | OD1 | ASP | A | 702 | 32.906 | 60.804 | −49.056 | 1.00 | 44.15 | A |
| ATOM | 5530 | OD2 | ASP | A | 702 | 33.624 | 59.822 | −47.221 | 1.00 | 44.63 | A |
| ATOM | 5531 | C | ASP | A | 702 | 29.698 | 61.859 | −46.105 | 1.00 | 40.87 | A |
| ATOM | 5532 | O | ASP | A | 702 | 28.556 | 62.159 | −46.457 | 1.00 | 41.18 | A |
| ATOM | 5533 | N | SER | A | 703 | 30.014 | 61.520 | −44.859 | 1.00 | 39.98 | A |
| ATOM | 5534 | CA | SER | A | 703 | 29.029 | 61.452 | −43.786 | 1.00 | 38.58 | A |
| ATOM | 5535 | CB | SER | A | 703 | 29.739 | 61.217 | −42.450 | 1.00 | 38.76 | A |
| ATOM | 5536 | OG | SER | A | 703 | 30.659 | 62.258 | −42.172 | 1.00 | 37.91 | A |
| ATOM | 5537 | C | SER | A | 703 | 28.147 | 62.693 | −43.683 | 1.00 | 37.58 | A |
| ATOM | 5538 | O | SER | A | 703 | 28.471 | 63.751 | −44.224 | 1.00 | 37.44 | A |
| ATOM | 5539 | N | PRO | A | 704 | 27.009 | 62.572 | −42.983 | 1.00 | 36.57 | A |
| ATOM | 5540 | CD | PRO | A | 704 | 26.426 | 61.309 | −42.488 | 1.00 | 36.72 | A |
| ATOM | 5541 | CA | PRO | A | 704 | 26.068 | 63.677 | −42.798 | 1.00 | 35.60 | A |
| ATOM | 5542 | CB | PRO | A | 704 | 24.751 | 62.956 | −42.571 | 1.00 | 36.25 | A |
| ATOM | 5543 | CG | PRO | A | 704 | 25.184 | 61.784 | −41.753 | 1.00 | 36.32 | A |
| ATOM | 5544 | C | PRO | A | 704 | 26.434 | 64.571 | −41.616 | 1.00 | 34.44 | A |
| ATOM | 5545 | O | PRO | A | 704 | 27.327 | 64.250 | −40.832 | 1.00 | 33.95 | A |
| ATOM | 5546 | N | HIS | A | 705 | 25.730 | 65.693 | −41.502 | 1.00 | 32.79 | A |
| ATOM | 5547 | CA | HIS | A | 705 | 25.946 | 66.640 | −40.416 | 1.00 | 30.84 | A |
| ATOM | 5548 | CB | HIS | A | 705 | 25.500 | 68.041 | −40.839 | 1.00 | 31.81 | A |
| ATOM | 5549 | CG | HIS | A | 705 | 26.333 | 68.634 | −41.932 | 1.00 | 33.35 | A |
| ATOM | 5550 | CD2 | HIS | A | 705 | 26.029 | 68.952 | −43.212 | 1.00 | 34.15 | A |
| ATOM | 5551 | ND1 | HIS | A | 705 | 27.664 | 68.951 | −41.764 | 1.00 | 33.69 | A |
| ATOM | 5552 | CE1 | HIS | A | 705 | 28.145 | 69.437 | −42.894 | 1.00 | 33.97 | A |
| ATOM | 5553 | NE2 | HIS | A | 705 | 27.173 | 69.448 | −43.789 | 1.00 | 34.80 | A |
| ATOM | 5554 | C | HIS | A | 705 | 25.136 | 66.186 | −39.209 | 1.00 | 28.81 | A |
| ATOM | 5555 | O | HIS | A | 705 | 23.952 | 66.491 | −39.096 | 1.00 | 28.15 | A |
| ATOM | 5556 | N | VAL | A | 706 | 25.781 | 65.452 | −38.310 | 1.00 | 26.49 | A |
| ATOM | 5557 | CA | VAL | A | 706 | 25.109 | 64.950 | −37.120 | 1.00 | 24.60 | A |
| ATOM | 5558 | CB | VAL | A | 706 | 25.994 | 63.920 | −36.384 | 1.00 | 23.73 | A |
| ATOM | 5559 | CG1 | VAL | A | 706 | 25.276 | 63.404 | −35.148 | 1.00 | 23.27 | A |
| ATOM | 5560 | CG2 | VAL | A | 706 | 26.335 | 62.774 | −37.320 | 1.00 | 23.50 | A |
| ATOM | 5561 | C | VAL | A | 706 | 24.757 | 66.075 | −36.154 | 1.00 | 23.78 | A |
| ATOM | 5562 | O | VAL | A | 706 | 25.622 | 66.845 | −35.745 | 1.00 | 23.66 | A |
| ATOM | 5563 | N | PRO | A | 707 | 23.473 | 66.187 | −35.780 | 1.00 | 23.08 | A |
| ATOM | 5564 | CD | PRO | A | 707 | 22.320 | 65.469 | −36.351 | 1.00 | 23.24 | A |
| ATOM | 5565 | CA | PRO | A | 707 | 23.025 | 67.229 | −34.853 | 1.00 | 22.31 | A |
| ATOM | 5566 | CB | PRO | A | 707 | 21.507 | 67.042 | −34.831 | 1.00 | 22.67 | A |
| ATOM | 5567 | CG | PRO | A | 707 | 21.213 | 66.478 | −36.188 | 1.00 | 22.98 | A |
| ATOM | 5568 | C | PRO | A | 707 | 23.635 | 67.086 | −33.456 | 1.00 | 21.45 | A |
| ATOM | 5569 | O | PRO | A | 707 | 23.396 | 66.099 | −32.761 | 1.00 | 21.52 | A |
| ATOM | 5570 | N | VAL | A | 708 | 24.433 | 68.073 | −33.063 | 1.00 | 20.14 | A |
| ATOM | 5571 | CA | VAL | A | 708 | 25.070 | 68.102 | −31.748 | 1.00 | 18.90 | A |
| ATOM | 5572 | CB | VAL | A | 708 | 26.532 | 67.600 | −31.807 | 1.00 | 18.48 | A |
| ATOM | 5573 | CG1 | VAL | A | 708 | 27.171 | 67.679 | −30.422 | 1.00 | 17.73 | A |
| ATOM | 5574 | CG2 | VAL | A | 708 | 26.569 | 66.170 | −32.319 | 1.00 | 17.97 | A |
| ATOM | 5575 | C | VAL | A | 708 | 25.046 | 69.571 | −31.349 | 1.00 | 19.08 | A |
| ATOM | 5576 | O | VAL | A | 708 | 25.819 | 70.374 | −31.863 | 1.00 | 18.79 | A |
| ATOM | 5577 | N | HIS | A | 709 | 24.142 | 69.922 | −30.445 | 1.00 | 19.13 | A |
| ATOM | 5578 | CA | HIS | A | 709 | 24.001 | 71.310 | −30.030 | 1.00 | 19.46 | A |
| ATOM | 5579 | CB | HIS | A | 709 | 22.541 | 71.758 | −30.180 | 1.00 | 21.51 | A |
| ATOM | 5580 | CG | HIS | A | 709 | 22.012 | 71.661 | −31.577 | 1.00 | 24.16 | A |
| ATOM | 5581 | CD2 | HIS | A | 709 | 21.797 | 70.590 | −32.378 | 1.00 | 25.73 | A |
| ATOM | 5582 | ND1 | HIS | A | 709 | 21.600 | 72.764 | −32.295 | 1.00 | 26.33 | A |
| ATOM | 5583 | CE1 | HIS | A | 709 | 21.152 | 72.377 | −33.477 | 1.00 | 26.24 | A |
| ATOM | 5584 | NE2 | HIS | A | 709 | 21.261 | 71.062 | −33.552 | 1.00 | 25.69 | A |
| ATOM | 5585 | C | HIS | A | 709 | 24.441 | 71.580 | −28.602 | 1.00 | 18.62 | A |
| ATOM | 5586 | O | HIS | A | 709 | 24.065 | 70.857 | −27.679 | 1.00 | 18.91 | A |
| ATOM | 5587 | N | PHE | A | 710 | 25.244 | 72.626 | −28.435 | 1.00 | 17.43 | A |
| ATOM | 5588 | CA | PHE | A | 710 | 25.705 | 73.045 | −27.120 | 1.00 | 16.63 | A |
| ATOM | 5589 | CB | PHE | A | 710 | 27.123 | 73.623 | −27.195 | 1.00 | 16.26 | A |
| ATOM | 5590 | CG | PHE | A | 710 | 28.204 | 72.681 | −26.716 | 1.00 | 16.17 | A |
| ATOM | 5591 | CD1 | PHE | A | 710 | 29.374 | 72.517 | −27.451 | 1.00 | 16.64 | A |
| ATOM | 5592 | CD2 | PHE | A | 710 | 28.069 | 71.994 | −25.512 | 1.00 | 16.37 | A |
| ATOM | 5593 | CE1 | PHE | A | 710 | 30.398 | 71.682 | −26.994 | 1.00 | 16.33 | A |
| ATOM | 5594 | CE2 | PHE | A | 710 | 29.086 | 71.157 | −25.046 | 1.00 | 16.04 | A |
| ATOM | 5595 | CZ | PHE | A | 710 | 30.251 | 71.003 | −25.790 | 1.00 | 16.18 | A |
| ATOM | 5596 | C | PHE | A | 710 | 24.730 | 74.130 | −26.678 | 1.00 | 17.27 | A |
| ATOM | 5597 | O | PHE | A | 710 | 24.296 | 74.963 | −27.487 | 1.00 | 16.24 | A |
| ATOM | 5598 | N | LYS | A | 711 | 24.380 | 74.114 | −25.399 | 1.00 | 16.58 | A |
| ATOM | 5599 | CA | LYS | A | 711 | 23.453 | 75.089 | −24.854 | 1.00 | 18.02 | A |
| ATOM | 5600 | CB | LYS | A | 711 | 22.016 | 74.577 | −25.000 | 1.00 | 19.78 | A |
| ATOM | 5601 | CG | LYS | A | 711 | 20.960 | 75.492 | −24.411 | 1.00 | 22.47 | A |
| ATOM | 5602 | CD | LYS | A | 711 | 19.566 | 74.874 | −24.524 | 1.00 | 24.01 | A |
| ATOM | 5603 | CE | LYS | A | 711 | 19.132 | 74.717 | −25.973 | 1.00 | 24.28 | A |
| ATOM | 5604 | NZ | LYS | A | 711 | 17.796 | 74.059 | −26.070 | 1.00 | 26.13 | A |
| ATOM | 5605 | C | LYS | A | 711 | 23.776 | 75.309 | −23.384 | 1.00 | 17.48 | A |
| ATOM | 5606 | O | LYS | A | 711 | 24.155 | 74.372 | −22.681 | 1.00 | 17.60 | A |
| ATOM | 5607 | N | PHE | A | 712 | 23.636 | 76.546 | −22.926 | 1.00 | 16.29 | A |
| ATOM | 5608 | CA | PHE | A | 712 | 23.898 | 76.862 | −21.532 | 1.00 | 15.83 | A |
| ATOM | 5609 | CB | PHE | A | 712 | 24.890 | 78.028 | −21.413 | 1.00 | 15.72 | A |
| ATOM | 5610 | CG | PHE | A | 712 | 26.312 | 77.644 | −21.718 | 1.00 | 15.16 | A |
| ATOM | 5611 | CD1 | PHE | A | 712 | 26.800 | 77.689 | −23.020 | 1.00 | 14.61 | A |
| ATOM | 5612 | CD2 | PHE | A | 712 | 27.149 | 77.191 | −20.703 | 1.00 | 14.74 | A |
| ATOM | 5613 | CE1 | PHE | A | 712 | 28.103 | 77.286 | −23.308 | 1.00 | 14.86 | A |
| ATOM | 5614 | CE2 | PHE | A | 712 | 28.453 | 76.785 | −20.977 | 1.00 | 14.53 | A |
| ATOM | 5615 | CZ | PHE | A | 712 | 28.932 | 76.831 | −22.282 | 1.00 | 14.63 | A |
| ATOM | 5616 | C | PHE | A | 712 | 22.590 | 77.209 | −20.845 | 1.00 | 15.49 | A |
| ATOM | 5617 | O | PHE | A | 712 | 21.801 | 78.000 | −21.361 | 1.00 | 15.25 | A |
| ATOM | 5618 | N | LEU | A | 713 | 22.347 | 76.594 | −19.693 | 1.00 | 15.13 | A |
| ATOM | 5619 | CA | LEU | A | 713 | 21.122 | 76.853 | −18.949 | 1.00 | 15.92 | A |
| ATOM | 5620 | CB | LEU | A | 713 | 20.165 | 75.654 | −19.040 | 1.00 | 15.54 | A |
| ATOM | 5621 | CG | LEU | A | 713 | 19.731 | 75.215 | −20.442 | 1.00 | 16.03 | A |
| ATOM | 5622 | CD1 | LEU | A | 713 | 20.687 | 74.147 | −20.966 | 1.00 | 16.71 | A |
| ATOM | 5623 | CD2 | LEU | A | 713 | 18.310 | 74.655 | −20.398 | 1.00 | 15.98 | A |
| ATOM | 5624 | C | LEU | A | 713 | 21.452 | 77.150 | −17.495 | 1.00 | 16.60 | A |
| ATOM | 5625 | O | LEU | A | 713 | 22.615 | 77.106 | −17.092 | 1.00 | 16.74 | A |
| ATOM | 5626 | N | LYS | A | 714 | 20.432 | 77.469 | −16.709 | 1.00 | 16.73 | A |
| ATOM | 5627 | CA | LYS | A | 714 | 20.648 | 77.766 | −15.307 | 1.00 | 17.78 | A |
| ATOM | 5628 | CB | LYS | A | 714 | 20.707 | 79.282 | −15.083 | 1.00 | 20.35 | A |
| ATOM | 5629 | CG | LYS | A | 714 | 19.521 | 80.049 | −15.640 | 1.00 | 23.01 | A |
| ATOM | 5630 | CD | LYS | A | 714 | 19.545 | 81.517 | −15.208 | 1.00 | 25.87 | A |
| ATOM | 5631 | CE | LYS | A | 714 | 20.797 | 82.237 | −15.693 | 1.00 | 27.66 | A |
| ATOM | 5632 | NZ | LYS | A | 714 | 20.843 | 83.662 | −15.245 | 1.00 | 29.31 | A |
| ATOM | 5633 | C | LYS | A | 714 | 19.585 | 77.162 | −14.405 | 1.00 | 17.94 | A |
| ATOM | 5634 | O | LYS | A | 714 | 18.399 | 77.120 | −14.752 | 1.00 | 17.86 | A |
| ATOM | 5635 | N | TYR | A | 715 | 20.034 | 76.668 | −13.255 | 1.00 | 17.22 | A |
| ATOM | 5636 | CA | TYR | A | 715 | 19.153 | 76.093 | −12.253 | 1.00 | 16.49 | A |
| ATOM | 5637 | CB | TYR | A | 715 | 19.738 | 74.804 | −11.658 | 1.00 | 15.95 | A |
| ATOM | 5638 | CG | TYR | A | 715 | 19.644 | 73.582 | −12.540 | 1.00 | 14.81 | A |
| ATOM | 5639 | CD1 | TYR | A | 715 | 20.718 | 73.182 | −13.334 | 1.00 | 15.39 | A |
| ATOM | 5640 | CE1 | TYR | A | 715 | 20.634 | 72.037 | −14.142 | 1.00 | 14.36 | A |
| ATOM | 5641 | CD2 | TYR | A | 715 | 18.479 | 72.815 | −12.572 | 1.00 | 14.77 | A |
| ATOM | 5642 | CE2 | TYR | A | 715 | 18.383 | 71.679 | −13.372 | 1.00 | 14.23 | A |
| ATOM | 5643 | CZ | TYR | A | 715 | 19.459 | 71.294 | −14.151 | 1.00 | 14.55 | A |
| ATOM | 5644 | OH | TYR | A | 715 | 19.356 | 70.167 | −14.936 | 1.00 | 13.86 | A |
| ATOM | 5645 | C | TYR | A | 715 | 19.068 | 77.128 | −11.145 | 1.00 | 17.13 | A |
| ATOM | 5646 | O | TYR | A | 715 | 20.021 | 77.874 | −10.916 | 1.00 | 17.00 | A |
| ATOM | 5647 | N | GLY | A | 716 | 17.936 | 77.170 | −10.455 | 1.00 | 17.07 | A |
| ATOM | 5648 | CA | GLY | A | 716 | 17.783 | 78.116 | −9.368 | 1.00 | 16.98 | A |
| ATOM | 5649 | C | GLY | A | 716 | 17.685 | 77.390 | −8.040 | 1.00 | 17.56 | A |
| ATOM | 5650 | O | GLY | A | 716 | 18.003 | 76.200 | −7.943 | 1.00 | 17.72 | A |
| ATOM | 5651 | N | VAL | A | 717 | 17.253 | 78.108 | −7.012 | 1.00 | 17.71 | A |
| ATOM | 5652 | CA | VAL | A | 717 | 17.099 | 77.536 | −5.683 | 1.00 | 18.46 | A |
| ATOM | 5653 | CB | VAL | A | 717 | 18.017 | 78.246 | −4.671 | 1.00 | 18.67 | A |
| ATOM | 5654 | CG1 | VAL | A | 717 | 17.805 | 77.675 | −3.284 | 1.00 | 20.04 | A |
| ATOM | 5655 | CG2 | VAL | A | 717 | 19.478 | 78.078 | −5.094 | 1.00 | 18.36 | A |
| ATOM | 5656 | C | VAL | A | 717 | 15.643 | 77.684 | −5.253 | 1.00 | 19.21 | A |
| ATOM | 5657 | O | VAL | A | 717 | 14.963 | 78.622 | −5.664 | 1.00 | 18.75 | A |
| ATOM | 5658 | N | ARG | A | 718 | 15.165 | 76.754 | −4.435 | 1.00 | 19.97 | A |
| ATOM | 5659 | CA | ARG | A | 718 | 13.781 | 76.791 | −3.976 | 1.00 | 21.50 | A |
| ATOM | 5660 | CB | ARG | A | 718 | 13.408 | 75.451 | −3.335 | 1.00 | 20.47 | A |
| ATOM | 5661 | CG | ARG | A | 718 | 13.489 | 74.288 | −4.302 | 1.00 | 20.11 | A |
| ATOM | 5662 | CD | ARG | A | 718 | 13.385 | 72.943 | −3.604 | 1.00 | 19.03 | A |
| ATOM | 5663 | NE | ARG | A | 718 | 13.656 | 71.860 | −4.544 | 1.00 | 19.26 | A |
| ATOM | 5664 | CZ | ARG | A | 718 | 13.681 | 70.569 | −4.228 | 1.00 | 19.63 | A |
| ATOM | 5665 | NH1 | ARG | A | 718 | 13.449 | 70.180 | −2.980 | 1.00 | 19.86 | A |
| ATOM | 5666 | NH2 | ARG | A | 718 | 13.944 | 69.668 | −5.164 | 1.00 | 19.58 | A |
| ATOM | 5667 | C | ARG | A | 718 | 13.524 | 77.924 | −2.992 | 1.00 | 22.88 | A |
| ATOM | 5668 | O | ARG | A | 718 | 14.349 | 78.214 | −2.130 | 1.00 | 22.82 | A |
| ATOM | 5669 | N | SER | A | 719 | 12.371 | 78.567 | −3.132 | 1.00 | 25.26 | A |
| ATOM | 5670 | CA | SER | A | 719 | 12.001 | 79.666 | −2.250 | 1.00 | 27.91 | A |
| ATOM | 5671 | CB | SER | A | 719 | 11.035 | 80.614 | −2.964 | 1.00 | 28.09 | A |
| ATOM | 5672 | OG | SER | A | 719 | 9.875 | 79.922 | −3.389 | 1.00 | 29.06 | A |
| ATOM | 5673 | C | SER | A | 719 | 11.346 | 79.111 | −0.990 | 1.00 | 29.36 | A |
| ATOM | 5674 | O | SER | A | 719 | 11.204 | 79.812 | 0.009 | 1.00 | 29.63 | A |
| ATOM | 5675 | N | HIS | A | 720 | 10.952 | 77.841 | −1.049 | 1.00 | 31.11 | A |
| ATOM | 5676 | CA | HIS | A | 720 | 10.311 | 77.173 | 0.077 | 1.00 | 32.47 | A |
| ATOM | 5677 | CB | HIS | A | 720 | 8.848 | 76.867 | −0.252 | 1.00 | 34.68 | A |
| ATOM | 5678 | CG | HIS | A | 720 | 8.066 | 78.062 | −0.697 | 1.00 | 37.29 | A |
| ATOM | 5679 | CD2 | HIS | A | 720 | 7.384 | 78.304 | −1.842 | 1.00 | 38.42 | A |
| ATOM | 5680 | ND1 | HIS | A | 720 | 7.922 | 79.190 | 0.081 | 1.00 | 38.37 | A |
| ATOM | 5681 | CE1 | HIS | A | 720 | 7.185 | 80.077 | −0.565 | 1.00 | 38.97 | A |
| ATOM | 5682 | NE2 | HIS | A | 720 | 6.846 | 79.564 | −1.734 | 1.00 | 39.06 | A |
| ATOM | 5683 | C | HIS | A | 720 | 11.026 | 75.865 | 0.403 | 1.00 | 31.94 | A |
| ATOM | 5684 | O | HIS | A | 720 | 11.453 | 75.142 | −0.497 | 1.00 | 32.28 | A |
| ATOM | 5685 | N | GLY | A | 721 | 11.153 | 75.569 | 1.692 | 1.00 | 30.87 | A |
| ATOM | 5686 | CA | GLY | A | 721 | 11.798 | 74.335 | 2.105 | 1.00 | 29.22 | A |
| ATOM | 5687 | C | GLY | A | 721 | 13.315 | 74.357 | 2.100 | 1.00 | 27.82 | A |
| ATOM | 5688 | O | GLY | A | 721 | 13.935 | 75.412 | 2.210 | 1.00 | 27.64 | A |
| ATOM | 5689 | N | ASP | A | 722 | 13.906 | 73.172 | 1.966 | 1.00 | 26.07 | A |
| ATOM | 5690 | CA | ASP | A | 722 | 15.356 | 73.002 | 1.960 | 1.00 | 24.10 | A |
| ATOM | 5691 | CB | ASP | A | 722 | 15.692 | 71.509 | 1.918 | 1.00 | 23.11 | A |
| ATOM | 5692 | CG | ASP | A | 722 | 15.151 | 70.755 | 3.125 | 1.00 | 22.96 | A |
| ATOM | 5693 | OD1 | ASP | A | 722 | 15.073 | 69.509 | 3.069 | 1.00 | 21.33 | A |
| ATOM | 5694 | OD2 | ASP | A | 722 | 14.814 | 71.412 | 4.134 | 1.00 | 22.49 | A |
| ATOM | 5695 | C | ASP | A | 722 | 16.029 | 73.717 | 0.795 | 1.00 | 23.18 | A |
| ATOM | 5696 | O | ASP | A | 722 | 15.590 | 73.611 | −0.348 | 1.00 | 22.51 | A |
| ATOM | 5697 | N | ARG | A | 723 | 17.101 | 74.444 | 1.096 | 1.00 | 22.44 | A |
| ATOM | 5698 | CA | ARG | A | 723 | 17.844 | 75.179 | 0.080 | 1.00 | 22.26 | A |
| ATOM | 5699 | CB | ARG | A | 723 | 18.173 | 76.595 | 0.567 | 1.00 | 24.76 | A |
| ATOM | 5700 | CG | ARG | A | 723 | 17.039 | 77.606 | 0.440 | 1.00 | 29.74 | A |
| ATOM | 5701 | CD | ARG | A | 723 | 15.975 | 77.422 | 1.506 | 1.00 | 33.28 | A |
| ATOM | 5702 | NE | ARG | A | 723 | 14.938 | 78.450 | 1.411 | 1.00 | 36.56 | A |
| ATOM | 5703 | CZ | ARG | A | 723 | 13.960 | 78.617 | 2.297 | 1.00 | 38.09 | A |
| ATOM | 5704 | NH1 | ARG | A | 723 | 13.874 | 77.822 | 3.358 | 1.00 | 39.31 | A |
| ATOM | 5705 | NH2 | ARG | A | 723 | 13.067 | 79.584 | 2.126 | 1.00 | 39.06 | A |
| ATOM | 5706 | C | ARG | A | 723 | 19.144 | 74.486 | −0.310 | 1.00 | 20.66 | A |
| ATOM | 5707 | O | ARG | A | 723 | 19.776 | 73.811 | 0.506 | 1.00 | 19.49 | A |
| ATOM | 5708 | N | SER | A | 724 | 19.537 | 74.665 | −1.567 | 1.00 | 18.62 | A |
| ATOM | 5709 | CA | SER | A | 724 | 20.771 | 74.087 | −2.075 | 1.00 | 17.82 | A |
| ATOM | 5710 | CB | SER | A | 724 | 20.882 | 74.309 | −3.586 | 1.00 | 16.97 | A |
| ATOM | 5711 | OG | SER | A | 724 | 19.807 | 73.698 | −4.279 | 1.00 | 17.89 | A |
| ATOM | 5712 | C | SER | A | 724 | 21.947 | 74.763 | −1.381 | 1.00 | 17.22 | A |
| ATOM | 5713 | O | SER | A | 724 | 21.888 | 75.952 | −1.053 | 1.00 | 17.26 | A |
| ATOM | 5714 | N | GLY | A | 725 | 23.013 | 73.999 | −1.164 | 1.00 | 16.49 | A |
| ATOM | 5715 | CA | GLY | A | 725 | 24.207 | 74.530 | −0.526 | 1.00 | 14.92 | A |
| ATOM | 5716 | C | GLY | A | 725 | 25.428 | 73.840 | −1.110 | 1.00 | 14.29 | A |
| ATOM | 5717 | O | GLY | A | 725 | 25.351 | 73.275 | −2.201 | 1.00 | 14.17 | A |
| ATOM | 5718 | N | ALA | A | 726 | 26.548 | 73.871 | −0.392 | 1.00 | 13.42 | A |
| ATOM | 5719 | CA | ALA | A | 726 | 27.778 | 73.241 | −0.869 | 1.00 | 12.82 | A |
| ATOM | 5720 | CB | ALA | A | 726 | 28.916 | 73.496 | 0.126 | 1.00 | 12.13 | A |
| ATOM | 5721 | C | ALA | A | 726 | 27.624 | 71.736 | −1.104 | 1.00 | 12.97 | A |
| ATOM | 5722 | O | ALA | A | 726 | 28.265 | 71.171 | −1.994 | 1.00 | 13.34 | A |
| ATOM | 5723 | N | TYR | A | 727 | 26.777 | 71.088 | −0.309 | 1.00 | 12.39 | A |
| ATOM | 5724 | CA | TYR | A | 727 | 26.574 | 69.647 | −0.440 | 1.00 | 12.54 | A |
| ATOM | 5725 | CB | TYR | A | 727 | 26.372 | 68.993 | 0.930 | 1.00 | 12.25 | A |
| ATOM | 5726 | CG | TYR | A | 727 | 27.389 | 69.346 | 1.980 | 1.00 | 11.70 | A |
| ATOM | 5727 | CD1 | TYR | A | 727 | 27.230 | 70.474 | 2.785 | 1.00 | 11.79 | A |
| ATOM | 5728 | CE1 | TYR | A | 727 | 28.162 | 70.785 | 3.776 | 1.00 | 12.23 | A |
| ATOM | 5729 | CD2 | TYR | A | 727 | 28.506 | 68.539 | 2.185 | 1.00 | 10.95 | A |
| ATOM | 5730 | CE2 | TYR | A | 727 | 29.441 | 68.840 | 3.165 | 1.00 | 10.75 | A |
| ATOM | 5731 | CZ | TYR | A | 727 | 29.265 | 69.959 | 3.959 | 1.00 | 11.16 | A |
| ATOM | 5732 | OH | TYR | A | 727 | 30.175 | 70.230 | 4.950 | 1.00 | 11.14 | A |
| ATOM | 5733 | C | TYR | A | 727 | 25.368 | 69.272 | −1.287 | 1.00 | 12.80 | A |
| ATOM | 5734 | O | TYR | A | 727 | 25.465 | 68.461 | −2.210 | 1.00 | 12.13 | A |
| ATOM | 5735 | N | LEU | A | 728 | 24.227 | 69.864 | −0.948 | 1.00 | 13.19 | A |
| ATOM | 5736 | CA | LEU | A | 728 | 22.965 | 69.563 | −1.608 | 1.00 | 13.62 | A |
| ATOM | 5737 | CB | LEU | A | 728 | 21.815 | 69.751 | −0.611 | 1.00 | 13.85 | A |
| ATOM | 5738 | CG | LEU | A | 728 | 21.981 | 69.108 | 0.769 | 1.00 | 12.94 | A |
| ATOM | 5739 | CD1 | LEU | A | 728 | 20.719 | 69.334 | 1.599 | 1.00 | 12.74 | A |
| ATOM | 5740 | CD2 | LEU | A | 728 | 22.260 | 67.627 | 0.608 | 1.00 | 13.66 | A |
| ATOM | 5741 | C | LEU | A | 728 | 22.616 | 70.325 | −2.879 | 1.00 | 14.23 | A |
| ATOM | 5742 | O | LEU | A | 728 | 22.853 | 71.527 | −2.992 | 1.00 | 14.66 | A |
| ATOM | 5743 | N | PHE | A | 729 | 22.038 | 69.592 | −3.828 | 1.00 | 14.78 | A |
| ATOM | 5744 | CA | PHE | A | 729 | 21.569 | 70.148 | −5.089 | 1.00 | 15.09 | A |
| ATOM | 5745 | CB | PHE | A | 729 | 22.097 | 69.338 | −6.278 | 1.00 | 14.90 | A |
| ATOM | 5746 | CG | PHE | A | 729 | 21.636 | 69.848 | −7.624 | 1.00 | 14.75 | A |
| ATOM | 5747 | CD1 | PHE | A | 729 | 21.579 | 68.992 | −8.719 | 1.00 | 14.83 | A |
| ATOM | 5748 | CD2 | PHE | A | 729 | 21.280 | 71.186 | −7.801 | 1.00 | 14.81 | A |
| ATOM | 5749 | CE1 | PHE | A | 729 | 21.174 | 69.456 | −9.973 | 1.00 | 14.77 | A |
| ATOM | 5750 | CE2 | PHE | A | 729 | 20.875 | 71.661 | −9.051 | 1.00 | 14.05 | A |
| ATOM | 5751 | CZ | PHE | A | 729 | 20.821 | 70.797 | −10.137 | 1.00 | 14.66 | A |
| ATOM | 5752 | C | PHE | A | 729 | 20.047 | 70.000 | −4.997 | 1.00 | 15.61 | A |
| ATOM | 5753 | O | PHE | A | 729 | 19.519 | 68.893 | −5.089 | 1.00 | 15.12 | A |
| ATOM | 5754 | N | LEU | A | 730 | 19.353 | 71.116 | −4.794 | 1.00 | 15.87 | A |
| ATOM | 5755 | CA | LEU | A | 730 | 17.899 | 71.115 | −4.673 | 1.00 | 16.28 | A |
| ATOM | 5756 | CB | LEU | A | 730 | 17.504 | 71.461 | −3.238 | 1.00 | 15.75 | A |
| ATOM | 5757 | CG | LEU | A | 730 | 17.891 | 70.417 | −2.186 | 1.00 | 16.05 | A |
| ATOM | 5758 | CD1 | LEU | A | 730 | 17.851 | 71.032 | −0.800 | 1.00 | 16.81 | A |
| ATOM | 5759 | CD2 | LEU | A | 730 | 16.947 | 69.228 | −2.281 | 1.00 | 15.75 | A |
| ATOM | 5760 | C | LEU | A | 730 | 17.325 | 72.146 | −5.631 | 1.00 | 16.38 | A |
| ATOM | 5761 | O | LEU | A | 730 | 16.851 | 73.203 | −5.212 | 1.00 | 16.56 | A |
| ATOM | 5762 | N | PRO | A | 731 | 17.353 | 71.843 | −6.937 | 1.00 | 16.67 | A |
| ATOM | 5763 | CD | PRO | A | 731 | 17.697 | 70.533 | −7.522 | 1.00 | 16.03 | A |
| ATOM | 5764 | CA | PRO | A | 731 | 16.843 | 72.752 | −7.964 | 1.00 | 16.82 | A |
| ATOM | 5765 | CB | PRO | A | 731 | 17.196 | 72.031 | −9.257 | 1.00 | 16.40 | A |
| ATOM | 5766 | CG | PRO | A | 731 | 17.024 | 70.590 | −8.872 | 1.00 | 16.72 | A |
| ATOM | 5767 | C | PRO | A | 731 | 15.356 | 73.060 | −7.873 | 1.00 | 17.55 | A |
| ATOM | 5768 | O | PRO | A | 731 | 14.557 | 72.231 | −7.429 | 1.00 | 16.89 | A |
| ATOM | 5769 | N | ASN | A | 732 | 14.997 | 74.268 | −8.294 | 1.00 | 18.54 | A |
| ATOM | 5770 | CA | ASN | A | 732 | 13.604 | 74.686 | −8.298 | 1.00 | 19.97 | A |
| ATOM | 5771 | CB | ASN | A | 732 | 13.494 | 76.193 | −8.029 | 1.00 | 21.38 | A |
| ATOM | 5772 | CG | ASN | A | 732 | 14.176 | 77.033 | −9.092 | 1.00 | 22.50 | A |
| ATOM | 5773 | OD1 | ASN | A | 732 | 15.257 | 76.694 | −9.569 | 1.00 | 23.48 | A |
| ATOM | 5774 | ND2 | ASN | A | 732 | 13.551 | 78.148 | −9.457 | 1.00 | 23.21 | A |
| ATOM | 5775 | C | ASN | A | 732 | 13.051 | 74.335 | −9.673 | 1.00 | 19.85 | A |
| ATOM | 5776 | O | ASN | A | 732 | 12.525 | 75.188 | −10.389 | 1.00 | 20.43 | A |
| ATOM | 5777 | N | GLY | A | 733 | 13.198 | 73.063 | −10.038 | 1.00 | 19.15 | A |
| ATOM | 5778 | CA | GLY | A | 733 | 12.711 | 72.584 | −11.319 | 1.00 | 19.07 | A |
| ATOM | 5779 | C | GLY | A | 733 | 13.785 | 72.480 | −12.384 | 1.00 | 19.05 | A |
| ATOM | 5780 | O | GLY | A | 733 | 14.950 | 72.804 | −12.126 | 1.00 | 18.33 | A |
| ATOM | 5781 | N | PRO | A | 734 | 13.424 | 72.020 | −13.596 | 1.00 | 18.74 | A |
| ATOM | 5782 | CD | PRO | A | 734 | 12.079 | 71.580 | −14.004 | 1.00 | 19.27 | A |
| ATOM | 5783 | CA | PRO | A | 734 | 14.372 | 71.878 | −14.704 | 1.00 | 18.72 | A |
| ATOM | 5784 | CB | PRO | A | 734 | 13.488 | 71.423 | −15.864 | 1.00 | 18.91 | A |
| ATOM | 5785 | CG | PRO | A | 734 | 12.381 | 70.691 | −15.187 | 1.00 | 19.56 | A |
| ATOM | 5786 | C | PRO | A | 734 | 15.040 | 73.216 | −14.994 | 1.00 | 18.56 | A |
| ATOM | 5787 | O | PRO | A | 734 | 14.472 | 74.272 | −14.716 | 1.00 | 17.70 | A |
| ATOM | 5788 | N | ALA | A | 735 | 16.236 | 73.163 | −15.569 | 1.00 | 18.32 | A |
| ATOM | 5789 | CA | ALA | A | 735 | 16.990 | 74.370 | −15.885 | 1.00 | 18.62 | A |
| ATOM | 5790 | CB | ALA | A | 735 | 18.389 | 73.996 | −16.359 | 1.00 | 17.27 | A |
| ATOM | 5791 | C | ALA | A | 735 | 16.298 | 75.230 | −16.939 | 1.00 | 19.06 | A |
| ATOM | 5792 | O | ALA | A | 735 | 15.526 | 74.731 | −17.756 | 1.00 | 19.40 | A |
| ATOM | 5793 | N | SER | A | 736 | 16.585 | 76.528 | −16.902 | 1.00 | 19.80 | A |
| ATOM | 5794 | CA | SER | A | 736 | 16.024 | 77.488 | −17.846 | 1.00 | 20.40 | A |
| ATOM | 5795 | CB | SER | A | 736 | 15.392 | 78.664 | −17.097 | 1.00 | 20.65 | A |
| ATOM | 5796 | OG | SER | A | 736 | 14.423 | 78.217 | −16.164 | 1.00 | 23.95 | A |
| ATOM | 5797 | C | SER | A | 736 | 17.168 | 78.000 | −18.716 | 1.00 | 20.53 | A |
| ATOM | 5798 | O | SER | A | 736 | 18.277 | 78.210 | −18.229 | 1.00 | 19.37 | A |
| ATOM | 5799 | N | PRO | A | 737 | 16.911 | 78.217 | −20.013 | 1.00 | 21.05 | A |
| ATOM | 5800 | CD | PRO | A | 737 | 15.647 | 78.006 | −20.737 | 1.00 | 21.76 | A |
| ATOM | 5801 | CA | PRO | A | 737 | 17.955 | 78.705 | −20.919 | 1.00 | 22.31 | A |
| ATOM | 5802 | CB | PRO | A | 737 | 17.227 | 78.812 | −22.261 | 1.00 | 22.46 | A |
| ATOM | 5803 | CG | PRO | A | 737 | 16.133 | 77.790 | −22.147 | 1.00 | 22.19 | A |
| ATOM | 5804 | C | PRO | A | 737 | 18.539 | 80.048 | −20.489 | 1.00 | 22.95 | A |
| ATOM | 5805 | O | PRO | A | 737 | 17.816 | 80.923 | −20.015 | 1.00 | 22.83 | A |
| ATOM | 5806 | N | VAL | A | 738 | 19.851 | 80.205 | −20.641 | 1.00 | 24.07 | A |
| ATOM | 5807 | CA | VAL | A | 738 | 20.498 | 81.466 | −20.301 | 1.00 | 24.73 | A |
| ATOM | 5808 | CB | VAL | A | 738 | 22.040 | 81.312 | −20.194 | 1.00 | 25.09 | A |
| ATOM | 5809 | CG1 | VAL | A | 738 | 22.700 | 82.688 | −20.102 | 1.00 | 24.86 | A |
| ATOM | 5810 | CG2 | VAL | A | 738 | 22.403 | 80.477 | −18.973 | 1.00 | 24.29 | A |
| ATOM | 5811 | C | VAL | A | 738 | 20.183 | 82.436 | −21.441 | 1.00 | 25.95 | A |
| ATOM | 5812 | O | VAL | A | 738 | 20.322 | 82.083 | −22.610 | 1.00 | 25.41 | A |
| ATOM | 5813 | N | GLU | A | 739 | 19.741 | 83.644 | −21.102 | 1.00 | 26.97 | A |
| ATOM | 5814 | CA | GLU | A | 739 | 19.428 | 84.650 | −22.114 | 1.00 | 28.25 | A |
| ATOM | 5815 | CB | GLU | A | 739 | 18.665 | 85.818 | −21.488 | 1.00 | 30.01 | A |
| ATOM | 5816 | CG | GLU | A | 739 | 17.303 | 85.431 | −20.940 | 1.00 | 33.60 | A |
| ATOM | 5817 | CD | GLU | A | 739 | 16.353 | 84.951 | −22.022 | 1.00 | 35.66 | A |
| ATOM | 5818 | OE1 | GLU | A | 739 | 15.220 | 84.545 | −21.681 | 1.00 | 37.20 | A |
| ATOM | 5819 | OE2 | GLU | A | 739 | 16.734 | 84.983 | −23.213 | 1.00 | 37.11 | A |
| ATOM | 5820 | C | GLU | A | 739 | 20.755 | 85.130 | −22.681 | 1.00 | 27.58 | A |
| ATOM | 5821 | O | GLU | A | 739 | 21.543 | 85.764 | −21.981 | 1.00 | 27.38 | A |
| ATOM | 5822 | N | LEU | A | 740 | 20.989 | 84.826 | −23.952 | 1.00 | 27.14 | A |
| ATOM | 5823 | CA | LEU | A | 740 | 22.244 | 85.170 | −24.610 | 1.00 | 26.75 | A |
| ATOM | 5824 | CB | LEU | A | 740 | 22.579 | 84.100 | −25.645 | 1.00 | 26.88 | A |
| ATOM | 5825 | CG | LEU | A | 740 | 22.553 | 82.653 | −25.153 | 1.00 | 26.56 | A |
| ATOM | 5826 | CD1 | LEU | A | 740 | 22.920 | 81.733 | −26.304 | 1.00 | 26.17 | A |
| ATOM | 5827 | CD2 | LEU | A | 740 | 23.520 | 82.479 | −23.985 | 1.00 | 26.74 | A |
| ATOM | 5828 | C | LEU | A | 740 | 22.323 | 86.532 | −25.282 | 1.00 | 26.81 | A |
| ATOM | 5829 | O | LEU | A | 740 | 23.419 | 87.060 | −25.475 | 1.00 | 26.49 | A |
| ATOM | 5830 | N | GLY | A | 741 | 21.177 | 87.100 | −25.642 | 1.00 | 26.45 | A |
| ATOM | 5831 | CA | GLY | A | 741 | 21.192 | 88.383 | −26.318 | 1.00 | 26.66 | A |
| ATOM | 5832 | C | GLY | A | 741 | 21.866 | 88.202 | −27.666 | 1.00 | 26.47 | A |
| ATOM | 5833 | O | GLY | A | 741 | 21.623 | 87.211 | −28.352 | 1.00 | 27.08 | A |
| ATOM | 5834 | N | GLN | A | 742 | 22.713 | 89.150 | −28.050 | 1.00 | 26.64 | A |
| ATOM | 5835 | CA | GLN | A | 742 | 23.431 | 89.070 | −29.319 | 1.00 | 26.29 | A |
| ATOM | 5836 | CB | GLN | A | 742 | 23.055 | 90.256 | −30.214 | 1.00 | 28.36 | A |
| ATOM | 5837 | CG | GLN | A | 742 | 21.562 | 90.324 | −30.534 | 1.00 | 31.41 | A |
| ATOM | 5838 | CD | GLN | A | 742 | 21.193 | 91.499 | −31.424 | 1.00 | 33.06 | A |
| ATOM | 5839 | OE1 | GLN | A | 742 | 21.654 | 91.603 | −32.562 | 1.00 | 35.27 | A |
| ATOM | 5840 | NE2 | GLN | A | 742 | 20.353 | 92.389 | −30.908 | 1.00 | 34.23 | A |
| ATOM | 5841 | C | GLN | A | 742 | 24.924 | 89.088 | −28.996 | 1.00 | 24.83 | A |
| ATOM | 5842 | O | GLN | A | 742 | 25.595 | 90.106 | −29.149 | 1.00 | 25.14 | A |
| ATOM | 5843 | N | PRO | A | 743 | 25.460 | 87.942 | −28.545 | 1.00 | 23.56 | A |
| ATOM | 5844 | CD | PRO | A | 743 | 24.758 | 86.644 | −28.504 | 1.00 | 22.94 | A |
| ATOM | 5845 | CA | PRO | A | 743 | 26.869 | 87.778 | −28.175 | 1.00 | 21.91 | A |
| ATOM | 5846 | CB | PRO | A | 743 | 26.910 | 86.355 | −27.629 | 1.00 | 22.16 | A |
| ATOM | 5847 | CG | PRO | A | 743 | 25.902 | 85.654 | −28.484 | 1.00 | 22.68 | A |
| ATOM | 5848 | C | PRO | A | 743 | 27.886 | 87.995 | −29.289 | 1.00 | 20.81 | A |
| ATOM | 5849 | O | PRO | A | 743 | 27.612 | 87.742 | −30.462 | 1.00 | 20.15 | A |
| ATOM | 5850 | N | VAL | A | 744 | 29.068 | 88.469 | −28.903 | 1.00 | 19.08 | A |
| ATOM | 5851 | CA | VAL | A | 744 | 30.146 | 88.704 | −29.849 | 1.00 | 17.97 | A |
| ATOM | 5852 | CB | VAL | A | 744 | 31.143 | 89.751 | −29.314 | 1.00 | 17.62 | A |
| ATOM | 5853 | CG1 | VAL | A | 744 | 32.305 | 89.900 | −30.280 | 1.00 | 16.50 | A |
| ATOM | 5854 | CG2 | VAL | A | 744 | 30.435 | 91.090 | −29.124 | 1.00 | 18.66 | A |
| ATOM | 5855 | C | VAL | A | 744 | 30.875 | 87.385 | −30.078 | 1.00 | 17.11 | A |
| ATOM | 5856 | O | VAL | A | 744 | 31.257 | 86.704 | −29.125 | 1.00 | 16.81 | A |
| ATOM | 5857 | N | VAL | A | 745 | 31.054 | 87.028 | −31.344 | 1.00 | 15.64 | A |
| ATOM | 5858 | CA | VAL | A | 745 | 31.719 | 85.784 | −31.712 | 1.00 | 14.94 | A |
| ATOM | 5859 | CB | VAL | A | 745 | 30.820 | 84.932 | −32.634 | 1.00 | 13.94 | A |
| ATOM | 5860 | CG1 | VAL | A | 745 | 31.534 | 83.643 | −33.011 | 1.00 | 14.63 | A |
| ATOM | 5861 | CG2 | VAL | A | 745 | 29.501 | 84.635 | −31.943 | 1.00 | 14.28 | A |
| ATOM | 5862 | C | VAL | A | 745 | 33.033 | 86.033 | −32.436 | 1.00 | 14.70 | A |
| ATOM | 5863 | O | VAL | A | 745 | 33.090 | 86.826 | −33.375 | 1.00 | 14.58 | A |
| ATOM | 5864 | N | LEU | A | 746 | 34.089 | 85.351 | −31.999 | 1.00 | 14.53 | A |
| ATOM | 5865 | CA | LEU | A | 746 | 35.402 | 85.494 | −32.619 | 1.00 | 14.40 | A |
| ATOM | 5866 | CB | LEU | A | 746 | 36.460 | 85.856 | −31.573 | 1.00 | 14.29 | A |
| ATOM | 5867 | CG | LEU | A | 746 | 37.910 | 85.863 | −32.082 | 1.00 | 14.37 | A |
| ATOM | 5868 | CD1 | LEU | A | 746 | 38.105 | 86.957 | −33.125 | 1.00 | 15.10 | A |
| ATOM | 5869 | CD2 | LEU | A | 746 | 38.853 | 86.084 | −30.908 | 1.00 | 15.27 | A |
| ATOM | 5870 | C | LEU | A | 746 | 35.815 | 84.204 | −33.315 | 1.00 | 14.39 | A |
| ATOM | 5871 | O | LEU | A | 746 | 35.920 | 83.152 | −32.683 | 1.00 | 13.94 | A |
| ATOM | 5872 | N | VAL | A | 747 | 36.066 | 84.298 | −34.615 | 1.00 | 14.21 | A |
| ATOM | 5873 | CA | VAL | A | 747 | 36.467 | 83.135 | −35.396 | 1.00 | 15.07 | A |
| ATOM | 5874 | CB | VAL | A | 747 | 35.556 | 82.952 | −36.639 | 1.00 | 14.86 | A |
| ATOM | 5875 | CG1 | VAL | A | 747 | 35.974 | 81.703 | −37.414 | 1.00 | 15.78 | A |
| ATOM | 5876 | CG2 | VAL | A | 747 | 34.098 | 82.851 | −36.205 | 1.00 | 14.92 | A |
| ATOM | 5877 | C | VAL | A | 747 | 37.909 | 83.263 | −35.861 | 1.00 | 15.47 | A |
| ATOM | 5878 | O | VAL | A | 747 | 38.272 | 84.217 | −36.553 | 1.00 | 16.02 | A |
| ATOM | 5879 | N | THR | A | 748 | 38.736 | 82.305 | −35.465 | 1.00 | 15.28 | A |
| ATOM | 5880 | CA | THR | A | 748 | 40.134 | 82.299 | −35.861 | 1.00 | 16.46 | A |
| ATOM | 5881 | CB | THR | A | 748 | 41.065 | 82.284 | −34.627 | 1.00 | 16.60 | A |
| ATOM | 5882 | OG1 | THR | A | 748 | 40.862 | 83.485 | −33.869 | 1.00 | 16.45 | A |
| ATOM | 5883 | CG2 | THR | A | 748 | 42.531 | 82.197 | −35.056 | 1.00 | 16.62 | A |
| ATOM | 5884 | C | THR | A | 748 | 40.361 | 81.048 | −36.696 | 1.00 | 17.60 | A |
| ATOM | 5885 | O | THR | A | 748 | 40.133 | 79.931 | −36.228 | 1.00 | 17.28 | A |
| ATOM | 5886 | N | LYS | A | 749 | 40.796 | 81.236 | −37.938 | 1.00 | 18.35 | A |
| ATOM | 5887 | CA | LYS | A | 749 | 41.031 | 80.112 | −38.829 | 1.00 | 19.29 | A |
| ATOM | 5888 | CB | LYS | A | 749 | 40.192 | 80.263 | −40.099 | 1.00 | 21.29 | A |
| ATOM | 5889 | CG | LYS | A | 749 | 40.406 | 79.139 | −41.100 | 1.00 | 23.49 | A |
| ATOM | 5890 | CD | LYS | A | 749 | 39.530 | 79.306 | −42.328 | 1.00 | 26.20 | A |
| ATOM | 5891 | CE | LYS | A | 749 | 39.772 | 78.172 | −43.314 | 1.00 | 27.68 | A |
| ATOM | 5892 | NZ | LYS | A | 749 | 38.949 | 78.318 | −44.543 | 1.00 | 30.91 | A |
| ATOM | 5893 | C | LYS | A | 749 | 42.498 | 79.971 | −39.202 | 1.00 | 19.15 | A |
| ATOM | 5894 | O | LYS | A | 749 | 43.095 | 80.878 | −39.791 | 1.00 | 18.79 | A |
| ATOM | 5895 | N | GLY | A | 750 | 43.069 | 78.821 | −38.862 | 1.00 | 18.42 | A |
| ATOM | 5896 | CA | GLY | A | 750 | 44.465 | 78.568 | −39.164 | 1.00 | 18.50 | A |
| ATOM | 5897 | C | GLY | A | 750 | 44.660 | 77.198 | −39.774 | 1.00 | 18.80 | A |
| ATOM | 5898 | O | GLY | A | 750 | 43.759 | 76.354 | −39.732 | 1.00 | 18.11 | A |
| ATOM | 5899 | N | LYS | A | 751 | 45.839 | 76.978 | −40.346 | 1.00 | 19.14 | A |
| ATOM | 5900 | CA | LYS | A | 751 | 46.161 | 75.706 | −40.977 | 1.00 | 19.90 | A |
| ATOM | 5901 | CB | LYS | A | 751 | 47.461 | 75.830 | −41.777 | 1.00 | 22.12 | A |
| ATOM | 5902 | CG | LYS | A | 751 | 47.381 | 76.754 | −42.990 | 1.00 | 26.04 | A |
| ATOM | 5903 | CD | LYS | A | 751 | 46.556 | 76.130 | −44.111 | 1.00 | 28.95 | A |
| ATOM | 5904 | CE | LYS | A | 751 | 46.615 | 76.970 | −45.385 | 1.00 | 29.97 | A |
| ATOM | 5905 | NZ | LYS | A | 751 | 45.821 | 76.357 | −46.492 | 1.00 | 31.69 | A |
| ATOM | 5906 | C | LYS | A | 751 | 46.315 | 74.589 | −39.949 | 1.00 | 18.94 | A |
| ATOM | 5907 | O | LYS | A | 751 | 45.995 | 73.435 | −40.227 | 1.00 | 18.88 | A |
| ATOM | 5908 | N | LEU | A | 752 | 46.805 | 74.937 | −38.764 | 1.00 | 17.91 | A |
| ATOM | 5909 | CA | LEU | A | 752 | 47.018 | 73.952 | −37.709 | 1.00 | 17.62 | A |
| ATOM | 5910 | CB | LEU | A | 752 | 48.396 | 74.157 | −37.073 | 1.00 | 17.88 | A |
| ATOM | 5911 | CG | LEU | A | 752 | 49.624 | 74.124 | −37.989 | 1.00 | 18.55 | A |
| ATOM | 5912 | CD1 | LEU | A | 752 | 50.882 | 74.300 | −37.146 | 1.00 | 19.24 | A |
| ATOM | 5913 | CD2 | LEU | A | 752 | 49.674 | 72.806 | −38.753 | 1.00 | 19.87 | A |
| ATOM | 5914 | C | LEU | A | 752 | 45.957 | 73.995 | −36.615 | 1.00 | 16.94 | A |
| ATOM | 5915 | O | LEU | A | 752 | 45.637 | 72.970 | −36.010 | 1.00 | 16.36 | A |
| ATOM | 5916 | N | GLU | A | 753 | 45.415 | 75.180 | −36.361 | 1.00 | 15.88 | A |
| ATOM | 5917 | CA | GLU | A | 753 | 44.409 | 75.339 | −35.322 | 1.00 | 16.00 | A |
| ATOM | 5918 | CB | GLU | A | 753 | 45.089 | 75.654 | −33.986 | 1.00 | 16.54 | A |
| ATOM | 5919 | CG | GLU | A | 753 | 44.133 | 75.776 | −32.803 | 1.00 | 19.19 | A |
| ATOM | 5920 | CD | GLU | A | 753 | 44.826 | 76.245 | −31.535 | 1.00 | 20.56 | A |
| ATOM | 5921 | OE1 | GLU | A | 753 | 45.182 | 77.438 | −31.454 | 1.00 | 21.29 | A |
| ATOM | 5922 | OE2 | GLU | A | 753 | 45.022 | 75.419 | −30.619 | 1.00 | 22.13 | A |
| ATOM | 5923 | C | GLU | A | 753 | 43.418 | 76.451 | −35.648 | 1.00 | 16.08 | A |
| ATOM | 5924 | O | GLU | A | 753 | 43.807 | 77.550 | −36.049 | 1.00 | 15.51 | A |
| ATOM | 5925 | N | SER | A | 754 | 42.137 | 76.155 | −35.474 | 1.00 | 15.21 | A |
| ATOM | 5926 | CA | SER | A | 754 | 41.086 | 77.133 | −35.710 | 1.00 | 14.74 | A |
| ATOM | 5927 | CB | SER | A | 754 | 40.266 | 76.761 | −36.945 | 1.00 | 15.36 | A |
| ATOM | 5928 | OG | SER | A | 754 | 41.069 | 76.794 | −38.111 | 1.00 | 15.30 | A |
| ATOM | 5929 | C | SER | A | 754 | 40.205 | 77.085 | −34.477 | 1.00 | 15.07 | A |
| ATOM | 5930 | O | SER | A | 754 | 40.272 | 76.124 | −33.707 | 1.00 | 15.07 | A |
| ATOM | 5931 | N | SER | A | 755 | 39.383 | 78.106 | −34.277 | 1.00 | 14.22 | A |
| ATOM | 5932 | CA | SER | A | 755 | 38.510 | 78.111 | −33.115 | 1.00 | 15.10 | A |
| ATOM | 5933 | CB | SER | A | 755 | 39.316 | 78.412 | −31.848 | 1.00 | 16.03 | A |
| ATOM | 5934 | OG | SER | A | 755 | 39.812 | 79.737 | −31.867 | 1.00 | 16.70 | A |
| ATOM | 5935 | C | SER | A | 755 | 37.378 | 79.114 | −33.230 | 1.00 | 14.43 | A |
| ATOM | 5936 | O | SER | A | 755 | 37.424 | 80.045 | −34.038 | 1.00 | 14.90 | A |
| ATOM | 5937 | N | VAL | A | 756 | 36.354 | 78.898 | −32.419 | 1.00 | 13.70 | A |
| ATOM | 5938 | CA | VAL | A | 756 | 35.208 | 79.784 | −32.360 | 1.00 | 14.05 | A |
| ATOM | 5939 | CB | VAL | A | 756 | 33.937 | 79.117 | −32.913 | 1.00 | 13.69 | A |
| ATOM | 5940 | CG1 | VAL | A | 756 | 32.736 | 80.036 | −32.701 | 1.00 | 14.07 | A |
| ATOM | 5941 | CG2 | VAL | A | 756 | 34.122 | 78.796 | −34.391 | 1.00 | 14.09 | A |
| ATOM | 5942 | C | VAL | A | 756 | 35.019 | 80.079 | −30.879 | 1.00 | 13.94 | A |
| ATOM | 5943 | O | VAL | A | 756 | 34.833 | 79.162 | −30.078 | 1.00 | 14.01 | A |
| ATOM | 5944 | N | SER | A | 757 | 35.085 | 81.357 | −30.522 | 1.00 | 13.67 | A |
| ATOM | 5945 | CA | SER | A | 757 | 34.936 | 81.783 | −29.136 | 1.00 | 14.33 | A |
| ATOM | 5946 | CB | SER | A | 757 | 36.245 | 82.408 | −28.642 | 1.00 | 14.45 | A |
| ATOM | 5947 | OG | SER | A | 757 | 37.343 | 81.536 | −28.866 | 1.00 | 17.98 | A |
| ATOM | 5948 | C | SER | A | 757 | 33.815 | 82.806 | −29.029 | 1.00 | 13.90 | A |
| ATOM | 5949 | O | SER | A | 757 | 33.703 | 83.688 | −29.872 | 1.00 | 14.57 | A |
| ATOM | 5950 | N | VAL | A | 758 | 32.985 | 82.690 | −27.997 | 1.00 | 13.86 | A |
| ATOM | 5951 | CA | VAL | A | 758 | 31.883 | 83.633 | −27.820 | 1.00 | 14.32 | A |
| ATOM | 5952 | CB | VAL | A | 758 | 30.539 | 83.031 | −28.334 | 1.00 | 14.99 | A |
| ATOM | 5953 | CG1 | VAL | A | 758 | 30.255 | 81.697 | −27.652 | 1.00 | 15.26 | A |
| ATOM | 5954 | CG2 | VAL | A | 758 | 29.400 | 84.010 | −28.089 | 1.00 | 14.66 | A |
| ATOM | 5955 | C | VAL | A | 758 | 31.737 | 84.071 | −26.366 | 1.00 | 14.81 | A |
| ATOM | 5956 | O | VAL | A | 758 | 31.849 | 83.259 | −25.439 | 1.00 | 14.83 | A |
| ATOM | 5957 | N | GLY | A | 759 | 31.514 | 85.369 | −26.176 | 1.00 | 14.65 | A |
| ATOM | 5958 | CA | GLY | A | 759 | 31.352 | 85.915 | −24.843 | 1.00 | 15.14 | A |
| ATOM | 5959 | C | GLY | A | 759 | 29.902 | 85.865 | −24.406 | 1.00 | 16.00 | A |
| ATOM | 5960 | O | GLY | A | 759 | 29.102 | 86.744 | −24.738 | 1.00 | 15.45 | A |
| ATOM | 5961 | N | LEU | A | 760 | 29.560 | 84.822 | −23.663 | 1.00 | 15.97 | A |
| ATOM | 5962 | CA | LEU | A | 760 | 28.205 | 84.640 | −23.168 | 1.00 | 17.13 | A |
| ATOM | 5963 | CB | LEU | A | 760 | 27.828 | 83.157 | −23.228 | 1.00 | 17.83 | A |
| ATOM | 5964 | CG | LEU | A | 760 | 27.974 | 82.466 | −24.585 | 1.00 | 17.97 | A |
| ATOM | 5965 | CD1 | LEU | A | 760 | 27.745 | 80.971 | −24.427 | 1.00 | 18.75 | A |
| ATOM | 5966 | CD2 | LEU | A | 760 | 26.981 | 83.061 | −25.575 | 1.00 | 19.05 | A |
| ATOM | 5967 | C | LEU | A | 760 | 28.147 | 85.117 | −21.726 | 1.00 | 17.20 | A |
| ATOM | 5968 | O | LEU | A | 760 | 29.181 | 85.275 | −21.076 | 1.00 | 17.55 | A |
| ATOM | 5969 | N | PRO | A | 761 | 26.939 | 85.365 | −21.203 | 1.00 | 17.67 | A |
| ATOM | 5970 | CD | PRO | A | 761 | 25.623 | 85.445 | −21.859 | 1.00 | 17.96 | A |
| ATOM | 5971 | CA | PRO | A | 761 | 26.863 | 85.817 | −19.811 | 1.00 | 18.19 | A |
| ATOM | 5972 | CB | PRO | A | 761 | 25.365 | 86.026 | −19.600 | 1.00 | 18.69 | A |
| ATOM | 5973 | CG | PRO | A | 761 | 24.888 | 86.419 | −20.969 | 1.00 | 18.84 | A |
| ATOM | 5974 | C | PRO | A | 761 | 27.434 | 84.735 | −18.889 | 1.00 | 18.29 | A |
| ATOM | 5975 | O | PRO | A | 761 | 26.957 | 83.597 | −18.892 | 1.00 | 18.07 | A |
| ATOM | 5976 | N | SER | A | 762 | 28.464 | 85.094 | −18.127 | 1.00 | 17.62 | A |
| ATOM | 5977 | CA | SER | A | 762 | 29.121 | 84.189 | −17.182 | 1.00 | 17.31 | A |
| ATOM | 5978 | CB | SER | A | 762 | 28.084 | 83.390 | −16.379 | 1.00 | 17.41 | A |
| ATOM | 5979 | OG | SER | A | 762 | 27.237 | 84.229 | −15.623 | 1.00 | 18.47 | A |
| ATOM | 5980 | C | SER | A | 762 | 30.094 | 83.196 | −17.808 | 1.00 | 17.10 | A |
| ATOM | 5981 | O | SER | A | 762 | 30.812 | 82.493 | −17.087 | 1.00 | 17.23 | A |
| ATOM | 5982 | N | VAL | A | 763 | 30.134 | 83.126 | −19.134 | 1.00 | 16.20 | A |
| ATOM | 5983 | CA | VAL | A | 763 | 31.013 | 82.160 | −19.782 | 1.00 | 16.03 | A |
| ATOM | 5984 | CB | VAL | A | 763 | 30.283 | 80.805 | −19.995 | 1.00 | 15.77 | A |
| ATOM | 5985 | CG1 | VAL | A | 763 | 31.236 | 79.785 | −20.606 | 1.00 | 15.66 | A |
| ATOM | 5986 | CG2 | VAL | A | 763 | 29.720 | 80.290 | −18.680 | 1.00 | 16.14 | A |
| ATOM | 5987 | C | VAL | A | 763 | 31.564 | 82.560 | −21.140 | 1.00 | 15.81 | A |
| ATOM | 5988 | O | VAL | A | 763 | 30.815 | 82.958 | −22.031 | 1.00 | 16.03 | A |
| ATOM | 5989 | N | VAL | A | 764 | 32.881 | 82.473 | −21.288 | 1.00 | 14.86 | A |
| ATOM | 5990 | CA | VAL | A | 764 | 33.496 | 82.722 | −22.580 | 1.00 | 14.90 | A |
| ATOM | 5991 | CB | VAL | A | 764 | 34.873 | 83.415 | −22.464 | 1.00 | 15.02 | A |
| ATOM | 5992 | CG1 | VAL | A | 764 | 35.533 | 83.497 | −23.838 | 1.00 | 15.39 | A |
| ATOM | 5993 | CG2 | VAL | A | 764 | 34.701 | 84.815 | −21.888 | 1.00 | 14.57 | A |
| ATOM | 5994 | C | VAL | A | 764 | 33.666 | 81.276 | −23.041 | 1.00 | 15.18 | A |
| ATOM | 5995 | O | VAL | A | 764 | 34.509 | 80.543 | −22.518 | 1.00 | 14.33 | A |
| ATOM | 5996 | N | HIS | A | 765 | 32.818 | 80.872 | −23.982 | 1.00 | 14.66 | A |
| ATOM | 5997 | CA | HIS | A | 765 | 32.788 | 79.515 | −24.524 | 1.00 | 14.46 | A |
| ATOM | 5998 | CB | HIS | A | 765 | 31.340 | 79.162 | −24.895 | 1.00 | 14.53 | A |
| ATOM | 5999 | CG | HIS | A | 765 | 31.153 | 77.751 | −25.359 | 1.00 | 15.89 | A |
| ATOM | 6000 | CD2 | HIS | A | 765 | 31.423 | 76.571 | −24.754 | 1.00 | 14.81 | A |
| ATOM | 6001 | ND1 | HIS | A | 765 | 30.614 | 77.437 | −26.589 | 1.00 | 16.76 | A |
| ATOM | 6002 | CE1 | HIS | A | 765 | 30.562 | 76.124 | −26.722 | 1.00 | 15.67 | A |
| ATOM | 6003 | NE2 | HIS | A | 765 | 31.048 | 75.575 | −25.623 | 1.00 | 17.15 | A |
| ATOM | 6004 | C | HIS | A | 765 | 33.684 | 79.424 | −25.752 | 1.00 | 14.53 | A |
| ATOM | 6005 | O | HIS | A | 765 | 33.570 | 80.235 | −26.670 | 1.00 | 13.37 | A |
| ATOM | 6006 | N | GLN | A | 766 | 34.567 | 78.431 | −25.775 | 1.00 | 14.08 | A |
| ATOM | 6007 | CA | GLN | A | 766 | 35.490 | 78.280 | −26.892 | 1.00 | 15.14 | A |
| ATOM | 6008 | CB | GLN | A | 766 | 36.898 | 78.699 | −26.460 | 1.00 | 16.53 | A |
| ATOM | 6009 | CG | GLN | A | 766 | 36.955 | 79.975 | −25.632 | 1.00 | 20.12 | A |
| ATOM | 6010 | CD | GLN | A | 766 | 38.142 | 79.989 | −24.678 | 1.00 | 23.07 | A |
| ATOM | 6011 | OE1 | GLN | A | 766 | 39.288 | 79.816 | −25.096 | 1.00 | 24.43 | A |
| ATOM | 6012 | NE2 | GLN | A | 766 | 37.868 | 80.191 | −23.386 | 1.00 | 22.97 | A |
| ATOM | 6013 | C | GLN | A | 766 | 35.557 | 76.859 | −27.435 | 1.00 | 14.87 | A |
| ATOM | 6014 | O | GLN | A | 766 | 35.689 | 75.900 | −26.671 | 1.00 | 14.51 | A |
| ATOM | 6015 | N | THR | A | 767 | 35.464 | 76.732 | −28.756 | 1.00 | 13.43 | A |
| ATOM | 6016 | CA | THR | A | 767 | 35.564 | 75.433 | −29.411 | 1.00 | 13.83 | A |
| ATOM | 6017 | CB | THR | A | 767 | 34.340 | 75.141 | −30.303 | 1.00 | 13.86 | A |
| ATOM | 6018 | OG1 | THR | A | 767 | 33.150 | 75.212 | −29.513 | 1.00 | 14.60 | A |
| ATOM | 6019 | CG2 | THR | A | 767 | 34.444 | 73.741 | −30.906 | 1.00 | 14.49 | A |
| ATOM | 6020 | C | THR | A | 767 | 36.828 | 75.495 | −30.262 | 1.00 | 14.12 | A |
| ATOM | 6021 | O | THR | A | 767 | 36.919 | 76.284 | −31.207 | 1.00 | 14.35 | A |
| ATOM | 6022 | N | ILE | A | 768 | 37.808 | 74.668 | −29.913 | 1.00 | 14.54 | A |
| ATOM | 6023 | CA | ILE | A | 768 | 39.088 | 74.651 | −30.612 | 1.00 | 15.59 | A |
| ATOM | 6024 | CB | ILE | A | 768 | 40.243 | 74.766 | −29.599 | 1.00 | 15.69 | A |
| ATOM | 6025 | CG2 | ILE | A | 768 | 41.561 | 74.972 | −30.327 | 1.00 | 17.18 | A |
| ATOM | 6026 | CG1 | ILE | A | 768 | 39.970 | 75.931 | −28.648 | 1.00 | 17.37 | A |
| ATOM | 6027 | CD1 | ILE | A | 768 | 40.946 | 76.027 | −27.489 | 1.00 | 18.23 | A |
| ATOM | 6028 | C | ILE | A | 768 | 39.274 | 73.391 | −31.448 | 1.00 | 15.73 | A |
| ATOM | 6029 | O | ILE | A | 768 | 39.008 | 72.279 | −30.984 | 1.00 | 15.43 | A |
| ATOM | 6030 | N | MET | A | 769 | 39.736 | 73.570 | −32.681 | 1.00 | 16.42 | A |
| ATOM | 6031 | CA | MET | A | 769 | 39.938 | 72.449 | −33.595 | 1.00 | 17.29 | A |
| ATOM | 6032 | CB | MET | A | 769 | 39.030 | 72.612 | −34.816 | 1.00 | 18.73 | A |
| ATOM | 6033 | CG | MET | A | 769 | 37.549 | 72.608 | −34.478 | 1.00 | 19.51 | A |
| ATOM | 6034 | SD | MET | A | 769 | 36.524 | 73.303 | −35.794 | 1.00 | 22.39 | A |
| ATOM | 6035 | CE | MET | A | 769 | 36.428 | 74.985 | −35.246 | 1.00 | 22.33 | A |
| ATOM | 6036 | C | MET | A | 769 | 41.384 | 72.320 | −34.050 | 1.00 | 17.69 | A |
| ATOM | 6037 | O | MET | A | 769 | 42.004 | 73.302 | −34.468 | 1.00 | 16.93 | A |
| ATOM | 6038 | N | ARG | A | 770 | 41.914 | 71.101 | −33.977 | 1.00 | 17.77 | A |
| ATOM | 6039 | CA | ARG | A | 770 | 43.289 | 70.849 | −34.378 | 1.00 | 18.83 | A |
| ATOM | 6040 | CB | ARG | A | 770 | 44.161 | 70.653 | −33.137 | 1.00 | 19.71 | A |
| ATOM | 6041 | CG | ARG | A | 770 | 44.144 | 71.853 | −32.207 | 1.00 | 22.50 | A |
| ATOM | 6042 | CD | ARG | A | 770 | 44.964 | 71.617 | −30.951 | 1.00 | 24.80 | A |
| ATOM | 6043 | NE | ARG | A | 770 | 44.861 | 72.752 | −30.040 | 1.00 | 27.76 | A |
| ATOM | 6044 | CZ | ARG | A | 770 | 45.414 | 72.799 | −28.833 | 1.00 | 29.18 | A |
| ATOM | 6045 | NE1 | ARG | A | 770 | 46.118 | 71.769 | −28.379 | 1.00 | 29.75 | A |
| ATOM | 6046 | NH2 | ARG | A | 770 | 45.262 | 73.880 | −28.078 | 1.00 | 30.25 | A |
| ATOM | 6047 | C | ARG | A | 770 | 43.424 | 69.645 | −35.305 | 1.00 | 18.94 | A |
| ATOM | 6048 | O | ARG | A | 770 | 44.527 | 69.147 | −35.528 | 1.00 | 19.08 | A |
| ATOM | 6049 | N | GLY | A | 771 | 42.302 | 69.177 | −35.839 | 1.00 | 19.13 | A |
| ATOM | 6050 | CA | GLY | A | 771 | 42.346 | 68.045 | −36.747 | 1.00 | 20.15 | A |
| ATOM | 6051 | C | GLY | A | 771 | 41.608 | 66.810 | −36.274 | 1.00 | 20.51 | A |
| ATOM | 6052 | O | GLY | A | 771 | 41.376 | 65.886 | −37.057 | 1.00 | 22.20 | A |
| ATOM | 6053 | N | GLY | A | 772 | 41.250 | 66.777 | −34.995 | 1.00 | 20.11 | A |
| ATOM | 6054 | CA | GLY | A | 772 | 40.530 | 65.631 | −34.468 | 1.00 | 19.12 | A |
| ATOM | 6055 | C | GLY | A | 772 | 39.341 | 66.092 | −33.651 | 1.00 | 18.40 | A |
| ATOM | 6056 | O | GLY | A | 772 | 38.737 | 67.125 | −33.957 | 1.00 | 17.47 | A |
| ATOM | 6057 | N | ALA | A | 773 | 38.994 | 65.334 | −32.617 | 1.00 | 17.19 | A |
| ATOM | 6058 | CA | ALA | A | 773 | 37.880 | 65.721 | −31.770 | 1.00 | 16.28 | A |
| ATOM | 6059 | CB | ALA | A | 773 | 37.730 | 64.744 | −30.609 | 1.00 | 16.74 | A |
| ATOM | 6060 | C | ALA | A | 773 | 38.208 | 67.118 | −31.249 | 1.00 | 15.85 | A |
| ATOM | 6061 | O | ALA | A | 773 | 39.344 | 67.396 | −30.858 | 1.00 | 15.72 | A |
| ATOM | 6062 | N | PRO | A | 774 | 37.220 | 68.021 | −31.252 | 1.00 | 14.95 | A |
| ATOM | 6063 | CD | PRO | A | 774 | 35.834 | 67.870 | −31.729 | 1.00 | 14.54 | A |
| ATOM | 6064 | CA | PRO | A | 774 | 37.465 | 69.382 | −30.769 | 1.00 | 14.51 | A |
| ATOM | 6065 | CB | PRO | A | 774 | 36.203 | 70.128 | −31.190 | 1.00 | 14.89 | A |
| ATOM | 6066 | CG | PRO | A | 774 | 35.147 | 69.068 | −31.100 | 1.00 | 15.90 | A |
| ATOM | 6067 | C | PRO | A | 774 | 37.696 | 69.461 | −29.264 | 1.00 | 14.10 | A |
| ATOM | 6068 | O | PRO | A | 774 | 37.293 | 68.574 | −28.509 | 1.00 | 13.79 | A |
| ATOM | 6069 | N | GLU | A | 775 | 38.366 | 70.528 | −28.842 | 1.00 | 13.81 | A |
| ATOM | 6070 | CA | GLU | A | 775 | 38.613 | 70.768 | −27.432 | 1.00 | 13.78 | A |
| ATOM | 6071 | CB | GLU | A | 775 | 40.059 | 71.216 | −27.187 | 1.00 | 14.79 | A |
| ATOM | 6072 | CG | GLU | A | 775 | 40.363 | 71.499 | −25.712 | 1.00 | 17.46 | A |
| ATOM | 6073 | CD | GLU | A | 775 | 41.780 | 71.993 | −25.473 | 1.00 | 19.38 | A |
| ATOM | 6074 | OE1 | GLU | A | 775 | 42.685 | 71.613 | −26.246 | 1.00 | 21.30 | A |
| ATOM | 6075 | OE2 | GLU | A | 775 | 41.993 | 72.746 | −24.496 | 1.00 | 20.86 | A |
| ATOM | 6076 | C | GLU | A | 775 | 37.664 | 71.902 | −27.074 | 1.00 | 13.37 | A |
| ATOM | 6077 | O | GLU | A | 775 | 37.471 | 72.828 | −27.863 | 1.00 | 13.38 | A |
| ATOM | 6078 | N | ILE | A | 776 | 37.051 | 71.818 | −25.904 | 1.00 | 13.11 | A |
| ATOM | 6079 | CA | ILE | A | 776 | 36.138 | 72.863 | −25.469 | 1.00 | 13.46 | A |
| ATOM | 6080 | CB | ILE | A | 776 | 34.744 | 72.301 | −25.117 | 1.00 | 14.14 | A |
| ATOM | 6081 | CG2 | ILE | A | 776 | 33.790 | 73.451 | −24.809 | 1.00 | 14.86 | A |
| ATOM | 6082 | CG1 | ILE | A | 776 | 34.217 | 71.423 | −26.256 | 1.00 | 14.67 | A |
| ATOM | 6083 | CD1 | ILE | A | 776 | 34.074 | 72.134 | −27.589 | 1.00 | 15.09 | A |
| ATOM | 6084 | C | ILE | A | 776 | 36.717 | 73.489 | −24.211 | 1.00 | 13.09 | A |
| ATOM | 6085 | O | ILE | A | 776 | 37.173 | 72.777 | −23.313 | 1.00 | 12.40 | A |
| ATOM | 6086 | N | ARG | A | 777 | 36.715 | 74.817 | −24.155 | 1.00 | 13.21 | A |
| ATOM | 6087 | CA | ARG | A | 777 | 37.210 | 75.527 | −22.981 | 1.00 | 12.69 | A |
| ATOM | 6088 | CB | ARG | A | 777 | 38.529 | 76.246 | −23.285 | 1.00 | 13.71 | A |
| ATOM | 6089 | CG | ARG | A | 777 | 39.679 | 75.327 | −23.657 | 1.00 | 13.57 | A |
| ATOM | 6090 | CD | ARG | A | 777 | 40.982 | 76.107 | −23.811 | 1.00 | 14.60 | A |
| ATOM | 6091 | NE | ARG | A | 777 | 42.053 | 75.272 | −24.350 | 1.00 | 14.70 | A |
| ATOM | 6092 | CZ | ARG | A | 777 | 43.222 | 75.739 | −24.782 | 1.00 | 16.35 | A |
| ATOM | 6093 | NH1 | ARG | A | 777 | 43.477 | 77.042 | −24.737 | 1.00 | 17.89 | A |
| ATOM | 6094 | NH2 | ARG | A | 777 | 44.133 | 74.906 | −25.272 | 1.00 | 16.86 | A |
| ATOM | 6095 | C | ARG | A | 777 | 36.165 | 76.552 | −22.567 | 1.00 | 12.89 | A |
| ATOM | 6096 | O | ARG | A | 777 | 35.654 | 77.303 | −23.404 | 1.00 | 12.83 | A |
| ATOM | 6097 | N | ASN | A | 778 | 35.834 | 76.560 | −21.281 | 1.00 | 12.52 | A |
| ATOM | 6098 | CA | ASN | A | 778 | 34.867 | 77.503 | −20.740 | 1.00 | 12.48 | A |
| ATOM | 6099 | CB | ASN | A | 778 | 33.681 | 76.786 | −20.080 | 1.00 | 13.02 | A |
| ATOM | 6100 | CG | ASN | A | 778 | 32.744 | 76.134 | −21.080 | 1.00 | 12.77 | A |
| ATOM | 6101 | OD1 | ASN | A | 778 | 32.722 | 76.494 | −22.259 | 1.00 | 12.70 | A |
| ATOM | 6102 | ND2 | ASN | A | 778 | 31.944 | 75.180 | −20.604 | 1.00 | 11.18 | A |
| ATOM | 6103 | C | ASN | A | 778 | 35.519 | 78.371 | −19.677 | 1.00 | 13.06 | A |
| ATOM | 6104 | O | ASN | A | 778 | 35.991 | 77.856 | −18.661 | 1.00 | 12.54 | A |
| ATOM | 6105 | N | LEU | A | 779 | 35.557 | 79.680 | −19.907 | 1.00 | 12.87 | A |
| ATOM | 6106 | CA | LEU | A | 779 | 36.100 | 80.590 | −18.905 | 1.00 | 13.87 | A |
| ATOM | 6107 | CB | LEU | A | 779 | 36.749 | 81.814 | −19.562 | 1.00 | 15.11 | A |
| ATOM | 6108 | CG | LEU | A | 779 | 37.244 | 82.906 | −18.603 | 1.00 | 16.41 | A |
| ATOM | 6109 | CD1 | LEU | A | 779 | 38.237 | 82.328 | −17.610 | 1.00 | 17.15 | A |
| ATOM | 6110 | CD2 | LEU | A | 779 | 37.882 | 84.031 | −19.403 | 1.00 | 17.46 | A |
| ATOM | 6111 | C | LEU | A | 779 | 34.857 | 80.992 | −18.121 | 1.00 | 13.84 | A |
| ATOM | 6112 | O | LEU | A | 779 | 34.120 | 81.894 | −18.519 | 1.00 | 14.16 | A |
| ATOM | 6113 | N | VAL | A | 780 | 34.627 | 80.298 | −17.012 | 1.00 | 14.09 | A |
| ATOM | 6114 | CA | VAL | A | 780 | 33.450 | 80.509 | −16.181 | 1.00 | 14.38 | A |
| ATOM | 6115 | CB | VAL | A | 780 | 32.975 | 79.160 | −15.578 | 1.00 | 13.50 | A |
| ATOM | 6116 | CG1 | VAL | A | 780 | 31.658 | 79.348 | −14.830 | 1.00 | 14.96 | A |
| ATOM | 6117 | CG2 | VAL | A | 780 | 32.813 | 78.131 | −16.681 | 1.00 | 12.78 | A |
| ATOM | 6118 | C | VAL | A | 780 | 33.612 | 81.519 | −15.046 | 1.00 | 14.62 | A |
| ATOM | 6119 | O | VAL | A | 780 | 34.436 | 81.342 | −14.156 | 1.00 | 14.42 | A |
| ATOM | 6120 | N | ASP | A | 781 | 32.810 | 82.577 | −15.096 | 1.00 | 15.08 | A |
| ATOM | 6121 | CA | ASP | A | 781 | 32.824 | 83.616 | −14.069 | 1.00 | 15.19 | A |
| ATOM | 6122 | CB | ASP | A | 781 | 33.503 | 84.886 | −14.582 | 1.00 | 16.51 | A |
| ATOM | 6123 | CG | ASP | A | 781 | 33.578 | 85.975 | −13.522 | 1.00 | 17.79 | A |
| ATOM | 6124 | OD1 | ASP | A | 781 | 34.045 | 87.088 | −13.847 | 1.00 | 19.59 | A |
| ATOM | 6125 | OD2 | ASP | A | 781 | 33.174 | 85.718 | −12.366 | 1.00 | 17.43 | A |
| ATOM | 6126 | C | ASP | A | 781 | 31.371 | 83.909 | −13.738 | 1.00 | 15.21 | A |
| ATOM | 6127 | O | ASP | A | 781 | 30.736 | 84.763 | −14.363 | 1.00 | 14.21 | A |
| ATOM | 6128 | N | ILE | A | 782 | 30.851 | 83.182 | −12.758 | 1.00 | 15.24 | A |
| ATOM | 6129 | CA | ILE | A | 782 | 29.467 | 83.324 | −12.344 | 1.00 | 16.76 | A |
| ATOM | 6130 | CB | ILE | A | 782 | 29.075 | 82.148 | −11.416 | 1.00 | 16.22 | A |
| ATOM | 6131 | CG2 | ILE | A | 782 | 29.673 | 82.352 | −10.025 | 1.00 | 15.94 | A |
| ATOM | 6132 | CG1 | ILE | A | 782 | 27.555 | 82.016 | −11.348 | 1.00 | 16.35 | A |
| ATOM | 6133 | CD1 | ILE | A | 782 | 27.095 | 80.690 | −10.765 | 1.00 | 14.49 | A |
| ATOM | 6134 | C | ILE | A | 782 | 29.203 | 84.673 | −11.664 | 1.00 | 18.22 | A |
| ATOM | 6135 | O | ILE | A | 782 | 28.070 | 84.988 | −11.311 | 1.00 | 19.23 | A |
| ATOM | 6136 | N | GLY | A | 783 | 30.257 | 85.466 | −11.493 | 1.00 | 19.97 | A |
| ATOM | 6137 | CA | GLY | A | 783 | 30.117 | 86.784 | −10.890 | 1.00 | 22.08 | A |
| ATOM | 6138 | C | GLY | A | 783 | 29.283 | 86.844 | −9.624 | 1.00 | 23.38 | A |
| ATOM | 6139 | O | GLY | A | 783 | 29.552 | 86.123 | −8.663 | 1.00 | 23.43 | A |
| ATOM | 6140 | N | SER | A | 784 | 28.266 | 87.702 | −9.616 | 1.00 | 24.74 | A |
| ATOM | 6141 | CA | SER | A | 784 | 27.412 | 87.840 | −8.439 | 1.00 | 26.45 | A |
| ATOM | 6142 | CB | SER | A | 784 | 27.329 | 89.311 | −8.014 | 1.00 | 27.09 | A |
| ATOM | 6143 | OG | SER | A | 784 | 26.712 | 90.107 | −9.012 | 1.00 | 29.22 | A |
| ATOM | 6144 | C | SER | A | 784 | 26.002 | 87.274 | −8.628 | 1.00 | 27.13 | A |
| ATOM | 6145 | O | SER | A | 784 | 25.067 | 87.676 | −7.933 | 1.00 | 27.56 | A |
| ATOM | 6146 | N | LEU | A | 785 | 25.853 | 86.339 | −9.563 | 1.00 | 26.95 | A |
| ATOM | 6147 | CA | LEU | A | 785 | 24.558 | 85.712 | −9.820 | 1.00 | 26.92 | A |
| ATOM | 6148 | CB | LEU | A | 785 | 24.587 | 84.955 | −11.152 | 1.00 | 27.50 | A |
| ATOM | 6149 | CG | LEU | A | 785 | 24.491 | 85.746 | −12.461 | 1.00 | 28.53 | A |
| ATOM | 6150 | CD1 | LEU | A | 785 | 25.503 | 86.875 | −12.478 | 1.00 | 29.75 | A |
| ATOM | 6151 | CD2 | LEU | A | 785 | 24.729 | 84.802 | −13.629 | 1.00 | 28.80 | A |
| ATOM | 6152 | C | LEU | A | 785 | 24.230 | 84.739 | −8.691 | 1.00 | 26.47 | A |
| ATOM | 6153 | O | LEU | A | 785 | 24.457 | 83.531 | −8.810 | 1.00 | 26.29 | A |
| ATOM | 6154 | N | ASP | A | 786 | 23.688 | 85.265 | −7.598 | 1.00 | 25.45 | A |
| ATOM | 6155 | CA | ASP | A | 786 | 23.348 | 84.438 | −6.446 | 1.00 | 24.78 | A |
| ATOM | 6156 | CB | ASP | A | 786 | 22.884 | 85.315 | −5.281 | 1.00 | 26.58 | A |
| ATOM | 6157 | CG | ASP | A | 786 | 23.875 | 86.409 | −4.947 | 1.00 | 28.38 | A |
| ATOM | 6158 | OD1 | ASP | A | 786 | 25.073 | 86.102 | −4.770 | 1.00 | 27.73 | A |
| ATOM | 6159 | OD2 | ASP | A | 786 | 23.449 | 87.581 | −4.863 | 1.00 | 30.70 | A |
| ATOM | 6160 | C | ASP | A | 786 | 22.284 | 83.383 | −6.731 | 1.00 | 22.89 | A |
| ATOM | 6161 | O | ASP | A | 786 | 21.401 | 83.573 | −7.572 | 1.00 | 22.07 | A |
| ATOM | 6162 | N | ASN | A | 787 | 22.380 | 82.274 | −6.006 | 1.00 | 21.19 | A |
| ATOM | 6163 | CA | ASN | A | 787 | 21.450 | 81.161 | −6.135 | 1.00 | 20.77 | A |
| ATOM | 6164 | CB | ASN | A | 787 | 20.110 | 81.522 | −5.495 | 1.00 | 21.66 | A |
| ATOM | 6165 | CG | ASN | A | 787 | 20.249 | 81.835 | −4.020 | 1.00 | 23.65 | A |
| ATOM | 6166 | OD1 | ASN | A | 787 | 20.912 | 81.103 | −3.283 | 1.00 | 24.06 | A |
| ATOM | 6167 | ND2 | ASN | A | 787 | 19.629 | 82.922 | −3.580 | 1.00 | 24.91 | A |
| ATOM | 6168 | C | ASN | A | 787 | 21.261 | 80.749 | −7.583 | 1.00 | 19.69 | A |
| ATOM | 6169 | O | ASN | A | 787 | 20.141 | 80.599 | −8.070 | 1.00 | 19.17 | A |
| ATOM | 6170 | N | THR | A | 788 | 22.381 | 80.556 | −8.265 | 1.00 | 18.19 | A |
| ATOM | 6171 | CA | THR | A | 788 | 22.365 | 80.155 | −9.658 | 1.00 | 17.22 | A |
| ATOM | 6172 | CB | THR | A | 788 | 22.704 | 81.349 | −10.579 | 1.00 | 18.00 | A |
| ATOM | 6173 | OG1 | THR | A | 788 | 21.749 | 82.399 | −10.370 | 1.00 | 18.28 | A |
| ATOM | 6174 | CG2 | THR | A | 788 | 22.669 | 80.926 | −12.044 | 1.00 | 18.47 | A |
| ATOM | 6175 | C | THR | A | 788 | 23.398 | 79.058 | −9.886 | 1.00 | 15.98 | A |
| ATOM | 6176 | O | THR | A | 788 | 24.492 | 79.096 | −9.322 | 1.00 | 15.00 | A |
| ATOM | 6177 | N | GLU | A | 789 | 23.030 | 78.074 | −10.695 | 1.00 | 14.52 | A |
| ATOM | 6178 | CA | GLU | A | 789 | 23.937 | 76.990 | −11.045 | 1.00 | 14.59 | A |
| ATOM | 6179 | CB | GLU | A | 789 | 23.469 | 75.661 | −10.433 | 1.00 | 14.05 | A |
| ATOM | 6180 | CG | GLU | A | 789 | 23.440 | 75.701 | −8.906 | 1.00 | 14.04 | A |
| ATOM | 6181 | CD | GLU | A | 789 | 23.457 | 74.328 | −8.256 | 1.00 | 14.08 | A |
| ATOM | 6182 | OE1 | GLU | A | 789 | 24.222 | 73.453 | −8.724 | 1.00 | 13.65 | A |
| ATOM | 6183 | OE2 | GLU | A | 789 | 22.719 | 74.136 | −7.262 | 1.00 | 13.58 | A |
| ATOM | 6184 | C | GLU | A | 789 | 23.928 | 76.939 | −12.565 | 1.00 | 14.52 | A |
| ATOM | 6185 | O | GLU | A | 789 | 22.884 | 76.737 | −13.186 | 1.00 | 14.93 | A |
| ATOM | 6186 | N | ILE | A | 790 | 25.094 | 77.161 | −13.160 | 1.00 | 13.89 | A |
| ATOM | 6187 | CA | ILE | A | 790 | 25.228 | 77.164 | −14.608 | 1.00 | 13.95 | A |
| ATOM | 6188 | CB | ILE | A | 790 | 26.320 | 78.148 | −15.056 | 1.00 | 14.54 | A |
| ATOM | 6189 | CG2 | ILE | A | 790 | 26.396 | 78.178 | −16.576 | 1.00 | 15.34 | A |
| ATOM | 6190 | CG1 | ILE | A | 790 | 26.012 | 79.547 | −14.508 | 1.00 | 16.25 | A |
| ATOM | 6191 | CD1 | ILE | A | 790 | 27.135 | 80.551 | −14.712 | 1.00 | 18.66 | A |
| ATOM | 6192 | C | ILE | A | 790 | 25.574 | 75.778 | −15.134 | 1.00 | 14.00 | A |
| ATOM | 6193 | O | ILE | A | 790 | 26.532 | 75.151 | −14.680 | 1.00 | 13.91 | A |
| ATOM | 6194 | N | VAL | A | 791 | 24.791 | 75.304 | −16.094 | 1.00 | 13.84 | A |
| ATOM | 6195 | CA | VAL | A | 791 | 25.024 | 73.991 | −16.668 | 1.00 | 13.77 | A |
| ATOM | 6196 | CB | VAL | A | 791 | 23.811 | 73.054 | −16.417 | 1.00 | 14.65 | A |
| ATOM | 6197 | CG1 | VAL | A | 791 | 22.581 | 73.575 | −17.164 | 1.00 | 14.60 | A |
| ATOM | 6198 | CG2 | VAL | A | 791 | 24.143 | 71.633 | −16.854 | 1.00 | 13.66 | A |
| ATOM | 6199 | C | VAL | A | 791 | 25.274 | 74.081 | −18.167 | 1.00 | 14.13 | A |
| ATOM | 6200 | O | VAL | A | 791 | 24.707 | 74.936 | −18.853 | 1.00 | 14.03 | A |
| ATOM | 6201 | N | MET | A | 792 | 26.153 | 73.218 | −18.664 | 1.00 | 13.29 | A |
| ATOM | 6202 | CA | MET | A | 792 | 26.438 | 73.164 | −20.092 | 1.00 | 13.85 | A |
| ATOM | 6203 | CB | MET | A | 792 | 27.941 | 73.138 | −20.365 | 1.00 | 14.34 | A |
| ATOM | 6204 | CG | MET | A | 792 | 28.273 | 72.961 | −21.841 | 1.00 | 14.06 | A |
| ATOM | 6205 | SD | MET | A | 792 | 30.043 | 73.066 | −22.209 | 1.00 | 15.67 | A |
| ATOM | 6206 | CE | MET | A | 792 | 30.667 | 71.618 | −21.346 | 1.00 | 15.89 | A |
| ATOM | 6207 | C | MET | A | 792 | 25.803 | 71.865 | −20.564 | 1.00 | 13.88 | A |
| ATOM | 6208 | O | MET | A | 792 | 26.139 | 70.791 | −20.067 | 1.00 | 13.81 | A |
| ATOM | 6209 | N | ARG | A | 793 | 24.884 | 71.965 | −21.518 | 1.00 | 14.28 | A |
| ATOM | 6210 | CA | ARG | A | 793 | 24.184 | 70.788 | −22.022 | 1.00 | 14.14 | A |
| ATOM | 6211 | CB | ARG | A | 793 | 22.674 | 70.941 | −21.766 | 1.00 | 14.51 | A |
| ATOM | 6212 | CG | ARG | A | 793 | 21.804 | 69.766 | −22.242 | 1.00 | 14.54 | A |
| ATOM | 6213 | CD | ARG | A | 793 | 20.322 | 70.029 | −21.946 | 1.00 | 15.61 | A |
| ATOM | 6214 | NE | ARG | A | 793 | 20.062 | 70.132 | −20.511 | 1.00 | 16.05 | A |
| ATOM | 6215 | CZ | ARG | A | 793 | 18.974 | 70.684 | −19.982 | 1.00 | 16.93 | A |
| ATOM | 6216 | NH1 | ARG | A | 793 | 18.031 | 71.190 | −20.768 | 1.00 | 16.82 | A |
| ATOM | 6217 | NH2 | ARG | A | 793 | 18.830 | 70.743 | −18.662 | 1.00 | 17.05 | A |
| ATOM | 6218 | C | ARG | A | 793 | 24.423 | 70.536 | −23.503 | 1.00 | 14.27 | A |
| ATOM | 6219 | O | ARG | A | 793 | 24.637 | 71.466 | −24.285 | 1.00 | 14.21 | A |
| ATOM | 6220 | N | LEU | A | 794 | 24.396 | 69.260 | −23.873 | 1.00 | 14.02 | A |
| ATOM | 6221 | CA | LEU | A | 794 | 24.556 | 68.837 | −25.257 | 1.00 | 14.74 | A |
| ATOM | 6222 | CB | LEU | A | 794 | 25.730 | 67.857 | −25.391 | 1.00 | 15.15 | A |
| ATOM | 6223 | CG | LEU | A | 794 | 27.138 | 68.460 | −25.460 | 1.00 | 15.18 | A |
| ATOM | 6224 | CD1 | LEU | A | 794 | 28.176 | 67.441 | −25.009 | 1.00 | 16.00 | A |
| ATOM | 6225 | CD2 | LEU | A | 794 | 27.414 | 68.917 | −26.885 | 1.00 | 16.18 | A |
| ATOM | 6226 | C | LEU | A | 794 | 23.253 | 68.147 | −25.658 | 1.00 | 15.38 | A |
| ATOM | 6227 | O | LEU | A | 794 | 22.765 | 67.270 | −24.941 | 1.00 | 14.50 | A |
| ATOM | 6228 | N | GLU | A | 795 | 22.680 | 68.561 | −26.787 | 1.00 | 15.73 | A |
| ATOM | 6229 | CA | GLU | A | 795 | 21.437 | 67.966 | −27.274 | 1.00 | 16.52 | A |
| ATOM | 6230 | CB | GLU | A | 795 | 20.368 | 69.054 | −27.464 | 1.00 | 17.12 | A |
| ATOM | 6231 | CG | GLU | A | 795 | 20.156 | 69.921 | −26.228 | 1.00 | 19.27 | A |
| ATOM | 6232 | CD | GLU | A | 795 | 19.114 | 71.015 | −26.423 | 1.00 | 21.04 | A |
| ATOM | 6233 | OE1 | GLU | A | 795 | 19.073 | 71.623 | −27.515 | 1.00 | 22.12 | A |
| ATOM | 6234 | OE2 | GLU | A | 795 | 18.348 | 71.281 | −25.475 | 1.00 | 21.33 | A |
| ATOM | 6235 | C | GLU | A | 795 | 21.710 | 67.244 | −28.597 | 1.00 | 16.30 | A |
| ATOM | 6236 | O | GLU | A | 795 | 22.263 | 67.829 | −29.526 | 1.00 | 16.34 | A |
| ATOM | 6237 | N | THR | A | 796 | 21.337 | 65.967 | −28.673 | 1.00 | 16.19 | A |
| ATOM | 6238 | CA | THR | A | 796 | 21.553 | 65.181 | −29.883 | 1.00 | 16.65 | A |
| ATOM | 6239 | CB | THR | A | 796 | 22.744 | 64.211 | −29.737 | 1.00 | 16.82 | A |
| ATOM | 6240 | OG1 | THR | A | 796 | 22.345 | 63.091 | −28.932 | 1.00 | 16.53 | A |
| ATOM | 6241 | CG2 | THR | A | 796 | 23.935 | 64.910 | −29.086 | 1.00 | 16.74 | A |
| ATOM | 6242 | C | THR | A | 796 | 20.333 | 64.329 | −30.221 | 1.00 | 17.22 | A |
| ATOM | 6243 | O | THR | A | 796 | 19.332 | 64.347 | −29.509 | 1.00 | 17.97 | A |
| ATOM | 6244 | N | HIS | A | 797 | 20.443 | 63.577 | −31.312 | 1.00 | 19.17 | A |
| ATOM | 6245 | CA | HIS | A | 797 | 19.377 | 62.691 | −31.769 | 1.00 | 20.27 | A |
| ATOM | 6246 | CB | HIS | A | 797 | 19.235 | 62.775 | −33.291 | 1.00 | 21.87 | A |
| ATOM | 6247 | CG | HIS | A | 797 | 18.600 | 64.043 | −33.769 | 1.00 | 22.65 | A |
| ATOM | 6248 | CD2 | HIS | A | 797 | 17.956 | 65.025 | −33.097 | 1.00 | 22.75 | A |
| ATOM | 6249 | ND1 | HIS | A | 797 | 18.574 | 64.407 | −35.099 | 1.00 | 23.52 | A |
| ATOM | 6250 | CE1 | HIS | A | 797 | 17.941 | 65.560 | −35.223 | 1.00 | 23.34 | A |
| ATOM | 6251 | NE2 | HIS | A | 797 | 17.556 | 65.956 | −34.023 | 1.00 | 22.42 | A |
| ATOM | 6252 | C | HIS | A | 797 | 19.677 | 61.251 | −31.368 | 1.00 | 20.49 | A |
| ATOM | 6253 | O | HIS | A | 797 | 18.952 | 60.332 | −31.743 | 1.00 | 21.41 | A |
| ATOM | 6254 | N | ILE | A | 798 | 20.749 | 61.059 | −30.605 | 1.00 | 19.36 | A |
| ATOM | 6255 | CA | ILE | A | 798 | 21.132 | 59.724 | −30.157 | 1.00 | 18.76 | A |
| ATOM | 6256 | CB | ILE | A | 798 | 22.422 | 59.778 | −29.302 | 1.00 | 18.71 | A |
| ATOM | 6257 | CG2 | ILE | A | 798 | 22.770 | 58.384 | −28.786 | 1.00 | 17.92 | A |
| ATOM | 6258 | CG1 | ILE | A | 798 | 23.570 | 60.333 | −30.154 | 1.00 | 18.53 | A |
| ATOM | 6259 | CD1 | ILE | A | 798 | 24.874 | 60.555 | −29.395 | 1.00 | 17.95 | A |
| ATOM | 6260 | C | ILE | A | 798 | 19.997 | 59.105 | −29.349 | 1.00 | 18.42 | A |
| ATOM | 6261 | O | ILE | A | 798 | 19.477 | 59.719 | −28.418 | 1.00 | 17.56 | A |
| ATOM | 6262 | N | ASP | A | 799 | 19.609 | 57.888 | −29.723 | 1.00 | 18.81 | A |
| ATOM | 6263 | CA | ASP | A | 799 | 18.522 | 57.182 | −29.056 | 1.00 | 18.57 | A |
| ATOM | 6264 | CB | ASP | A | 799 | 17.872 | 56.196 | −30.031 | 1.00 | 20.01 | A |
| ATOM | 6265 | CG | ASP | A | 799 | 16.543 | 55.678 | −29.530 | 1.00 | 21.36 | A |
| ATOM | 6266 | OD1 | ASP | A | 799 | 16.070 | 54.651 | −30.053 | 1.00 | 22.33 | A |
| ATOM | 6267 | OD2 | ASP | A | 799 | 15.968 | 56.302 | −28.616 | 1.00 | 21.98 | A |
| ATOM | 6268 | C | ASP | A | 799 | 19.024 | 56.429 | −27.828 | 1.00 | 18.37 | A |
| ATOM | 6269 | O | ASP | A | 799 | 18.977 | 55.199 | −27.777 | 1.00 | 17.79 | A |
| ATOM | 6270 | N | SER | A | 800 | 19.493 | 57.175 | −26.834 | 1.00 | 17.69 | A |
| ATOM | 6271 | CA | SER | A | 800 | 20.023 | 56.584 | −25.610 | 1.00 | 17.20 | A |
| ATOM | 6272 | CB | SER | A | 800 | 21.013 | 57.556 | −24.959 | 1.00 | 16.54 | A |
| ATOM | 6273 | OG | SER | A | 800 | 20.383 | 58.792 | −24.673 | 1.00 | 16.23 | A |
| ATOM | 6274 | C | SER | A | 800 | 18.947 | 56.193 | −24.597 | 1.00 | 16.97 | A |
| ATOM | 6275 | O | SER | A | 800 | 19.195 | 55.375 | −23.711 | 1.00 | 16.98 | A |
| ATOM | 6276 | N | GLY | A | 801 | 17.760 | 56.780 | −24.719 | 1.00 | 16.72 | A |
| ATOM | 6277 | CA | GLY | A | 801 | 16.684 | 56.458 | −23.798 | 1.00 | 16.49 | A |
| ATOM | 6278 | C | GLY | A | 801 | 16.937 | 56.957 | −22.387 | 1.00 | 15.93 | A |
| ATOM | 6279 | O | GLY | A | 801 | 17.034 | 58.162 | −22.162 | 1.00 | 16.54 | A |
| ATOM | 6280 | N | ASP | A | 802 | 17.042 | 56.035 | −21.433 | 1.00 | 15.86 | A |
| ATOM | 6281 | CA | ASP | A | 802 | 17.290 | 56.402 | −20.042 | 1.00 | 15.52 | A |
| ATOM | 6282 | CB | ASP | A | 802 | 16.203 | 55.808 | −19.125 | 1.00 | 16.28 | A |
| ATOM | 6283 | CG | ASP | A | 802 | 16.093 | 54.290 | −19.229 | 1.00 | 17.22 | A |
| ATOM | 6284 | OD1 | ASP | A | 802 | 15.201 | 53.720 | −18.559 | 1.00 | 17.37 | A |
| ATOM | 6285 | OD2 | ASP | A | 802 | 16.884 | 53.661 | −19.967 | 1.00 | 17.32 | A |
| ATOM | 6286 | C | ASP | A | 802 | 18.672 | 55.947 | −19.571 | 1.00 | 15.32 | A |
| ATOM | 6287 | O | ASP | A | 802 | 18.969 | 55.979 | −18.378 | 1.00 | 14.86 | A |
| ATOM | 6288 | N | ILE | A | 803 | 19.514 | 55.542 | −20.517 | 1.00 | 14.31 | A |
| ATOM | 6289 | CA | ILE | A | 803 | 20.862 | 55.069 | −20.207 | 1.00 | 14.43 | A |
| ATOM | 6290 | CB | ILE | A | 803 | 21.166 | 53.753 | −20.968 | 1.00 | 14.45 | A |
| ATOM | 6291 | CG2 | ILE | A | 803 | 22.583 | 53.274 | −20.656 | 1.00 | 13.83 | A |
| ATOM | 6292 | CG1 | ILE | A | 803 | 20.139 | 52.681 | −20.593 | 1.00 | 15.02 | A |
| ATOM | 6293 | CD1 | ILE | A | 803 | 20.150 | 52.286 | −19.127 | 1.00 | 15.71 | A |
| ATOM | 6294 | C | ILE | A | 803 | 21.981 | 56.061 | −20.540 | 1.00 | 14.06 | A |
| ATOM | 6295 | O | ILE | A | 803 | 21.923 | 56.781 | −21.533 | 1.00 | 13.86 | A |
| ATOM | 6296 | N | PHE | A | 804 | 22.998 | 56.093 | −19.686 | 1.00 | 13.59 | A |
| ATOM | 6297 | CA | PHE | A | 804 | 24.166 | 56.931 | −19.915 | 1.00 | 13.28 | A |
| ATOM | 6298 | CB | PHE | A | 804 | 23.886 | 58.420 | −19.614 | 1.00 | 12.58 | A |
| ATOM | 6299 | CG | PHE | A | 804 | 23.523 | 58.726 | −18.184 | 1.00 | 12.47 | A |
| ATOM | 6300 | CD1 | PHE | A | 804 | 24.455 | 59.316 | −17.327 | 1.00 | 12.56 | A |
| ATOM | 6301 | CD2 | PHE | A | 804 | 22.231 | 58.500 | −17.714 | 1.00 | 12.59 | A |
| ATOM | 6302 | CE1 | PHE | A | 804 | 24.100 | 59.682 | −16.025 | 1.00 | 12.22 | A |
| ATOM | 6303 | CE2 | PHE | A | 804 | 21.866 | 58.859 | −16.416 | 1.00 | 12.70 | A |
| ATOM | 6304 | CZ | PHE | A | 804 | 22.804 | 59.455 | −15.569 | 1.00 | 13.23 | A |
| ATOM | 6305 | C | PHE | A | 804 | 25.304 | 56.377 | −19.074 | 1.00 | 13.15 | A |
| ATOM | 6306 | O | PHE | A | 804 | 25.084 | 55.547 | −18.196 | 1.00 | 13.40 | A |
| ATOM | 6307 | N | TYR | A | 805 | 26.523 | 56.804 | −19.366 | 1.00 | 13.12 | A |
| ATOM | 6308 | CA | TYR | A | 805 | 27.674 | 56.305 | −18.633 | 1.00 | 12.37 | A |
| ATOM | 6309 | CB | TYR | A | 805 | 28.549 | 55.447 | −19.552 | 1.00 | 12.55 | A |
| ATOM | 6310 | CG | TYR | A | 805 | 27.851 | 54.219 | −20.112 | 1.00 | 14.19 | A |
| ATOM | 6311 | CD1 | TYR | A | 805 | 26.860 | 54.335 | −21.090 | 1.00 | 14.01 | A |
| ATOM | 6312 | CE1 | TYR | A | 805 | 26.225 | 53.203 | −21.615 | 1.00 | 14.29 | A |
| ATOM | 6313 | CD2 | TYR | A | 805 | 28.190 | 52.941 | −19.667 | 1.00 | 14.18 | A |
| ATOM | 6314 | CE2 | TYR | A | 805 | 27.560 | 51.803 | −20.183 | 1.00 | 14.39 | A |
| ATOM | 6315 | CZ | TYR | A | 805 | 26.583 | 51.941 | −21.156 | 1.00 | 14.54 | A |
| ATOM | 6316 | OH | TYR | A | 805 | 25.973 | 50.820 | −21.675 | 1.00 | 14.20 | A |
| ATOM | 6317 | C | TYR | A | 805 | 28.509 | 57.431 | −18.050 | 1.00 | 12.21 | A |
| ATOM | 6318 | O | TYR | A | 805 | 28.649 | 58.494 | −18.657 | 1.00 | 11.85 | A |
| ATOM | 6319 | N | THR | A | 806 | 29.045 | 57.189 | −16.858 | 1.00 | 11.34 | A |
| ATOM | 6320 | CA | THR | A | 806 | 29.898 | 58.151 | −16.180 | 1.00 | 11.39 | A |
| ATOM | 6321 | CB | THR | A | 806 | 29.140 | 58.881 | −15.055 | 1.00 | 11.82 | A |
| ATOM | 6322 | OG1 | THR | A | 806 | 28.769 | 57.946 | −14.032 | 1.00 | 12.67 | A |
| ATOM | 6323 | CG2 | THR | A | 806 | 27.877 | 59.535 | −15.614 | 1.00 | 10.84 | A |
| ATOM | 6324 | C | THR | A | 806 | 31.058 | 57.343 | −15.605 | 1.00 | 11.45 | A |
| ATOM | 6325 | O | THR | A | 806 | 30.934 | 56.133 | −15.407 | 1.00 | 11.87 | A |
| ATOM | 6326 | N | ASP | A | 807 | 32.189 | 57.990 | −15.352 | 1.00 | 10.78 | A |
| ATOM | 6327 | CA | ASP | A | 807 | 33.327 | 57.262 | −14.822 | 1.00 | 10.94 | A |
| ATOM | 6328 | CB | ASP | A | 807 | 34.630 | 57.740 | −15.470 | 1.00 | 10.56 | A |
| ATOM | 6329 | CG | ASP | A | 807 | 35.086 | 59.091 | −14.953 | 1.00 | 10.21 | A |
| ATOM | 6330 | OD1 | ASP | A | 807 | 36.235 | 59.183 | −14.480 | 1.00 | 11.32 | A |
| ATOM | 6331 | OD2 | ASP | A | 807 | 34.308 | 60.061 | −15.026 | 1.00 | 11.19 | A |
| ATOM | 6332 | C | ASP | A | 807 | 33.438 | 57.385 | −13.314 | 1.00 | 10.70 | A |
| ATOM | 6333 | O | ASP | A | 807 | 32.855 | 58.278 | −12.699 | 1.00 | 9.93 | A |
| ATOM | 6334 | N | LEU | A | 808 | 34.182 | 56.456 | −12.729 | 1.00 | 10.38 | A |
| ATOM | 6335 | CA | LEU | A | 808 | 34.418 | 56.452 | −11.299 | 1.00 | 10.47 | A |
| ATOM | 6336 | CB | LEU | A | 808 | 33.963 | 55.124 | −10.676 | 1.00 | 10.91 | A |
| ATOM | 6337 | CG | LEU | A | 808 | 32.445 | 54.927 | −10.532 | 1.00 | 10.80 | A |
| ATOM | 6338 | CD1 | LEU | A | 808 | 32.126 | 53.458 | −10.277 | 1.00 | 12.22 | A |
| ATOM | 6339 | CD2 | LEU | A | 808 | 31.917 | 55.782 | −9.391 | 1.00 | 11.26 | A |
| ATOM | 6340 | C | LEU | A | 808 | 35.910 | 56.655 | −11.087 | 1.00 | 10.25 | A |
| ATOM | 6341 | O | LEU | A | 808 | 36.722 | 55.806 | −11.462 | 1.00 | 10.25 | A |
| ATOM | 6342 | N | ASN | A | 809 | 36.261 | 57.810 | −10.527 | 1.00 | 9.42 | A |
| ATOM | 6343 | CA | ASN | A | 809 | 37.647 | 58.149 | −10.219 | 1.00 | 9.53 | A |
| ATOM | 6344 | CB | ASN | A | 809 | 38.121 | 57.262 | −9.073 | 1.00 | 8.78 | A |
| ATOM | 6345 | CG | ASN | A | 809 | 37.128 | 57.226 | −7.933 | 1.00 | 8.98 | A |
| ATOM | 6346 | OD1 | ASN | A | 809 | 37.110 | 58.114 | −7.075 | 1.00 | 11.42 | A |
| ATOM | 6347 | ND2 | ASN | A | 809 | 36.272 | 56.215 | −7.934 | 1.00 | 7.45 | A |
| ATOM | 6348 | C | ASN | A | 809 | 38.623 | 58.046 | −11.386 | 1.00 | 9.64 | A |
| ATOM | 6349 | O | ASN | A | 809 | 39.807 | 57.785 | −11.177 | 1.00 | 9.79 | A |
| ATOM | 6350 | N | GLY | A | 810 | 38.133 | 58.261 | −12.604 | 1.00 | 10.08 | A |
| ATOM | 6351 | CA | GLY | A | 810 | 38.997 | 58.182 | −13.774 | 1.00 | 10.96 | A |
| ATOM | 6352 | C | GLY | A | 810 | 39.586 | 56.797 | −13.984 | 1.00 | 12.28 | A |
| ATOM | 6353 | O | GLY | A | 810 | 40.611 | 56.633 | −14.659 | 1.00 | 13.07 | A |
| ATOM | 6354 | N | LEU | A | 811 | 38.923 | 55.795 | −13.419 | 1.00 | 11.50 | A |
| ATOM | 6355 | CA | LEU | A | 811 | 39.384 | 54.414 | −13.501 | 1.00 | 11.55 | A |
| ATOM | 6356 | CB | LEU | A | 811 | 39.490 | 53.835 | −12.085 | 1.00 | 11.82 | A |
| ATOM | 6357 | CG | LEU | A | 811 | 39.816 | 52.344 | −11.968 | 1.00 | 13.81 | A |
| ATOM | 6358 | CD1 | LEU | A | 811 | 41.218 | 52.097 | −12.510 | 1.00 | 14.31 | A |
| ATOM | 6359 | CD2 | LEU | A | 811 | 39.706 | 51.896 | −10.515 | 1.00 | 13.02 | A |
| ATOM | 6360 | C | LEU | A | 811 | 38.511 | 53.487 | −14.344 | 1.00 | 11.64 | A |
| ATOM | 6361 | O | LEU | A | 811 | 39.022 | 52.644 | −15.086 | 1.00 | 12.98 | A |
| ATOM | 6362 | N | GLN | A | 812 | 37.197 | 53.647 | −14.235 | 1.00 | 10.70 | A |
| ATOM | 6363 | CA | GLN | A | 812 | 36.261 | 52.779 | −14.942 | 1.00 | 11.31 | A |
| ATOM | 6364 | CB | GLN | A | 812 | 35.885 | 51.613 | −14.031 | 1.00 | 12.03 | A |
| ATOM | 6365 | CG | GLN | A | 812 | 35.287 | 52.098 | −12.693 | 1.00 | 12.84 | A |
| ATOM | 6366 | CD | GLN | A | 812 | 34.982 | 50.970 | −11.722 | 1.00 | 14.63 | A |
| ATOM | 6367 | OE1 | GLN | A | 812 | 33.988 | 50.258 | −11.867 | 1.00 | 15.09 | A |
| ATOM | 6368 | NE2 | GLN | A | 812 | 35.843 | 50.804 | −10.723 | 1.00 | 14.88 | A |
| ATOM | 6369 | C | GLN | A | 812 | 34.995 | 53.534 | −15.292 | 1.00 | 11.14 | A |
| ATOM | 6370 | O | GLN | A | 812 | 34.692 | 54.549 | −14.670 | 1.00 | 11.87 | A |
| ATOM | 6371 | N | PHE | A | 813 | 34.259 | 53.037 | −16.282 | 1.00 | 10.54 | A |
| ATOM | 6372 | CA | PHE | A | 813 | 32.999 | 53.652 | −16.666 | 1.00 | 10.98 | A |
| ATOM | 6373 | CB | PHE | A | 813 | 32.957 | 53.952 | −18.170 | 1.00 | 11.42 | A |
| ATOM | 6374 | CG | PHE | A | 813 | 33.707 | 55.197 | −18.544 | 1.00 | 11.91 | A |
| ATOM | 6375 | CD1 | PHE | A | 813 | 35.098 | 55.202 | −18.578 | 1.00 | 11.51 | A |
| ATOM | 6376 | CD2 | PHE | A | 813 | 33.025 | 56.385 | −18.795 | 1.00 | 12.99 | A |
| ATOM | 6377 | CE1 | PHE | A | 813 | 35.803 | 56.372 | −18.851 | 1.00 | 11.25 | A |
| ATOM | 6378 | CE2 | PHE | A | 813 | 33.722 | 57.562 | −19.070 | 1.00 | 12.39 | A |
| ATOM | 6379 | CZ | PHE | A | 813 | 35.114 | 57.550 | −19.096 | 1.00 | 11.92 | A |
| ATOM | 6380 | C | PHE | A | 813 | 31.876 | 52.714 | −16.261 | 1.00 | 11.59 | A |
| ATOM | 6381 | O | PHE | A | 813 | 31.889 | 51.510 | −16.573 | 1.00 | 11.72 | A |
| ATOM | 6382 | N | ILE | A | 814 | 30.914 | 53.269 | −15.539 | 1.00 | 11.18 | A |
| ATOM | 6383 | CA | ILE | A | 814 | 29.799 | 52.493 | −15.037 | 1.00 | 11.06 | A |
| ATOM | 6384 | CB | ILE | A | 814 | 29.722 | 52.625 | −13.494 | 1.00 | 11.10 | A |
| ATOM | 6385 | CG2 | ILE | A | 814 | 29.416 | 54.066 | −13.103 | 1.00 | 11.45 | A |
| ATOM | 6386 | CG1 | ILE | A | 814 | 28.669 | 51.664 | −12.931 | 1.00 | 11.83 | A |
| ATOM | 6387 | CD1 | ILE | A | 814 | 28.662 | 51.592 | −11.402 | 1.00 | 11.69 | A |
| ATOM | 6388 | C | ILE | A | 814 | 28.487 | 52.933 | −15.681 | 1.00 | 11.26 | A |
| ATOM | 6389 | O | ILE | A | 814 | 28.261 | 54.118 | −15.923 | 1.00 | 10.34 | A |
| ATOM | 6390 | N | LYS | A | 815 | 27.629 | 51.960 | −15.967 | 1.00 | 11.46 | A |
| ATOM | 6391 | CA | LYS | A | 815 | 26.343 | 52.232 | −16.593 | 1.00 | 11.88 | A |
| ATOM | 6392 | CB | LYS | A | 815 | 25.751 | 50.926 | −17.135 | 1.00 | 12.12 | A |
| ATOM | 6393 | CG | LYS | A | 815 | 24.392 | 51.047 | −17.815 | 1.00 | 14.24 | A |
| ATOM | 6394 | CD | LYS | A | 815 | 23.997 | 49.696 | −18.420 | 1.00 | 16.46 | A |
| ATOM | 6395 | CE | LYS | A | 815 | 22.678 | 49.769 | −19.164 | 1.00 | 19.21 | A |
| ATOM | 6396 | NZ | LYS | A | 815 | 22.321 | 48.454 | −19.780 | 1.00 | 20.77 | A |
| ATOM | 6397 | C | LYS | A | 815 | 25.384 | 52.879 | −15.603 | 1.00 | 11.66 | A |
| ATOM | 6398 | O | LYS | A | 815 | 25.252 | 52.423 | −14.468 | 1.00 | 12.12 | A |
| ATOM | 6399 | N | ARG | A | 816 | 24.733 | 53.953 | −16.043 | 1.00 | 11.64 | A |
| ATOM | 6400 | CA | ARG | A | 816 | 23.768 | 54.689 | −15.232 | 1.00 | 11.48 | A |
| ATOM | 6401 | CB | ARG | A | 816 | 24.129 | 56.178 | −15.165 | 1.00 | 11.74 | A |
| ATOM | 6402 | CG | ARG | A | 816 | 25.517 | 56.492 | −14.641 | 1.00 | 12.05 | A |
| ATOM | 6403 | CD | ARG | A | 816 | 25.687 | 55.935 | −13.245 | 1.00 | 12.44 | A |
| ATOM | 6404 | NE | ARG | A | 816 | 26.852 | 56.488 | −12.560 | 1.00 | 11.57 | A |
| ATOM | 6405 | CZ | ARG | A | 816 | 27.222 | 56.119 | −11.339 | 1.00 | 12.04 | A |
| ATOM | 6406 | NH1 | ARG | A | 816 | 26.517 | 55.201 | −10.691 | 1.00 | 10.85 | A |
| ATOM | 6407 | NH2 | ARG | A | 816 | 28.281 | 56.672 | −10.764 | 1.00 | 10.30 | A |
| ATOM | 6408 | C | ARG | A | 816 | 22.400 | 54.586 | −15.884 | 1.00 | 11.32 | A |
| ATOM | 6409 | O | ARG | A | 816 | 22.300 | 54.506 | −17.105 | 1.00 | 10.94 | A |
| ATOM | 6410 | N | ARG | A | 817 | 21.351 | 54.582 | −15.073 | 1.00 | 11.27 | A |
| ATOM | 6411 | CA | ARG | A | 817 | 20.000 | 54.551 | −15.615 | 1.00 | 12.22 | A |
| ATOM | 6412 | CB | ARG | A | 817 | 19.293 | 53.210 | −15.334 | 1.00 | 12.89 | A |
| ATOM | 6413 | CG | ARG | A | 817 | 17.827 | 53.209 | −15.802 | 1.00 | 13.67 | A |
| ATOM | 6414 | CD | ARG | A | 817 | 17.111 | 51.865 | −15.632 | 1.00 | 14.62 | A |
| ATOM | 6415 | NE | ARG | A | 817 | 17.626 | 50.837 | −16.532 | 1.00 | 15.59 | A |
| ATOM | 6416 | CZ | ARG | A | 817 | 18.366 | 49.804 | −16.144 | 1.00 | 16.50 | A |
| ATOM | 6417 | NH1 | ARG | A | 817 | 18.682 | 49.654 | −14.864 | 1.00 | 17.15 | A |
| ATOM | 6418 | NH2 | ARG | A | 817 | 18.793 | 48.919 | −17.035 | 1.00 | 16.79 | A |
| ATOM | 6419 | C | ARG | A | 817 | 19.213 | 55.687 | −14.981 | 1.00 | 12.55 | A |
| ATOM | 6420 | O | ARG | A | 817 | 19.037 | 55.724 | −13.764 | 1.00 | 12.99 | A |
| ATOM | 6421 | N | ARG | A | 818 | 18.778 | 56.636 | −15.805 | 1.00 | 12.88 | A |
| ATOM | 6422 | CA | ARG | A | 818 | 17.983 | 57.752 | −15.319 | 1.00 | 13.50 | A |
| ATOM | 6423 | CB | ARG | A | 818 | 17.625 | 58.699 | −16.469 | 1.00 | 13.33 | A |
| ATOM | 6424 | CG | ARG | A | 818 | 16.925 | 59.978 | −16.019 | 1.00 | 14.76 | A |
| ATOM | 6425 | CD | ARG | A | 818 | 16.277 | 60.714 | −17.186 | 1.00 | 15.79 | A |
| ATOM | 6426 | NE | ARG | A | 818 | 14.988 | 60.122 | −17.540 | 1.00 | 18.26 | A |
| ATOM | 6427 | CZ | ARG | A | 818 | 14.714 | 59.543 | −18.707 | 1.00 | 18.39 | A |
| ATOM | 6428 | NH1 | ARG | A | 818 | 15.638 | 59.467 | −19.659 | 1.00 | 16.54 | A |
| ATOM | 6429 | NH2 | ARG | A | 818 | 13.508 | 59.034 | −18.920 | 1.00 | 19.11 | A |
| ATOM | 6430 | C | ARG | A | 818 | 16.705 | 57.143 | −14.754 | 1.00 | 13.51 | A |
| ATOM | 6431 | O | ARG | A | 818 | 16.060 | 56.330 | −15.421 | 1.00 | 14.71 | A |
| ATOM | 6432 | N | LEU | A | 819 | 16.350 | 57.524 | −13.532 | 1.00 | 13.42 | A |
| ATOM | 6433 | CA | LEU | A | 819 | 15.146 | 57.007 | −12.884 | 1.00 | 14.74 | A |
| ATOM | 6434 | CB | LEU | A | 819 | 15.511 | 56.302 | −11.571 | 1.00 | 14.98 | A |
| ATOM | 6435 | CG | LEU | A | 819 | 16.430 | 55.080 | −11.695 | 1.00 | 15.76 | A |
| ATOM | 6436 | CD1 | LEU | A | 819 | 16.851 | 54.606 | −10.312 | 1.00 | 15.83 | A |
| ATOM | 6437 | CD2 | LEU | A | 819 | 15.714 | 53.972 | −12.456 | 1.00 | 15.44 | A |
| ATOM | 6438 | C | LEU | A | 819 | 14.180 | 58.150 | −12.604 | 1.00 | 15.35 | A |
| ATOM | 6439 | O | LEU | A | 819 | 14.431 | 58.995 | −11.745 | 1.00 | 15.18 | A |
| ATOM | 6440 | N | ASP | A | 820 | 13.068 | 58.176 | −13.329 | 1.00 | 16.48 | A |
| ATOM | 6441 | CA | ASP | A | 820 | 12.100 | 59.238 | −13.138 | 1.00 | 17.08 | A |
| ATOM | 6442 | CD | ASP | A | 820 | 11.111 | 59.271 | −14.306 | 1.00 | 18.98 | A |
| ATOM | 6443 | CG | ASP | A | 820 | 11.798 | 59.547 | −15.635 | 1.00 | 20.10 | A |
| ATOM | 6444 | OD1 | ASP | A | 820 | 12.762 | 60.341 | −15.650 | 1.00 | 21.45 | A |
| ATOM | 6445 | OD2 | ASP | A | 820 | 11.377 | 58.980 | −16.665 | 1.00 | 22.62 | A |
| ATOM | 6446 | C | ASP | A | 820 | 11.375 | 59.106 | −11.805 | 1.00 | 17.02 | A |
| ATOM | 6447 | O | ASP | A | 820 | 10.710 | 60.042 | −11.360 | 1.00 | 16.56 | A |
| ATOM | 6448 | N | LYS | A | 821 | 11.516 | 57.953 | −11.157 | 1.00 | 16.60 | A |
| ATOM | 6449 | CA | LYS | A | 821 | 10.875 | 57.756 | −9.862 | 1.00 | 16.60 | A |
| ATOM | 6450 | CB | LYS | A | 821 | 10.794 | 56.265 | −9.505 | 1.00 | 17.21 | A |
| ATOM | 6451 | CG | LYS | A | 821 | 12.139 | 55.579 | −9.317 | 1.00 | 17.29 | A |
| ATOM | 6452 | CD | LYS | A | 821 | 11.970 | 54.073 | −9.157 | 1.00 | 16.45 | A |
| ATOM | 6453 | CE | LYS | A | 821 | 13.320 | 53.374 | −9.053 | 1.00 | 16.98 | A |
| ATOM | 6454 | NZ | LYS | A | 821 | 13.184 | 51.888 | −9.114 | 1.00 | 15.59 | A |
| ATOM | 6455 | C | LYS | A | 821 | 11.667 | 58.516 | −8.798 | 1.00 | 16.66 | A |
| ATOM | 6456 | O | LYS | A | 821 | 11.233 | 58.638 | −7.655 | 1.00 | 16.84 | A |
| ATOM | 6457 | N | LEU | A | 822 | 12.831 | 59.030 | −9.187 | 1.00 | 15.85 | A |
| ATOM | 6458 | CA | LEU | A | 822 | 13.667 | 59.804 | −8.274 | 1.00 | 15.43 | A |
| ATOM | 6459 | CB | LEU | A | 822 | 15.088 | 59.235 | −8.234 | 1.00 | 14.68 | A |
| ATOM | 6460 | CG | LEU | A | 822 | 15.220 | 57.795 | −7.723 | 1.00 | 14.77 | A |
| ATOM | 6461 | CD1 | LEU | A | 822 | 16.684 | 57.370 | −7.747 | 1.00 | 14.13 | A |
| ATOM | 6462 | CD2 | LEU | A | 822 | 14.657 | 57.704 | −6.308 | 1.00 | 13.61 | A |
| ATOM | 6463 | C | LEU | A | 822 | 13.712 | 61.257 | −8.742 | 1.00 | 15.52 | A |
| ATOM | 6464 | O | LEU | A | 822 | 13.538 | 61.538 | −9.928 | 1.00 | 15.57 | A |
| ATOM | 6465 | N | PRO | A | 823 | 13.940 | 62.202 | −7.815 | 1.00 | 15.92 | A |
| ATOM | 6466 | CD | PRO | A | 823 | 14.103 | 62.039 | −6.360 | 1.00 | 15.59 | A |
| ATOM | 6467 | CA | PRO | A | 823 | 13.999 | 63.617 | −8.200 | 1.00 | 15.73 | A |
| ATOM | 6468 | CB | PRO | A | 823 | 14.021 | 64.342 | −6.857 | 1.00 | 15.56 | A |
| ATOM | 6469 | CG | PRO | A | 823 | 14.721 | 63.362 | −5.955 | 1.00 | 16.99 | A |
| ATOM | 6470 | C | PRO | A | 823 | 15.221 | 63.927 | −9.070 | 1.00 | 15.83 | A |
| ATOM | 6471 | O | PRO | A | 823 | 16.194 | 63.176 | −9.086 | 1.00 | 16.38 | A |
| ATOM | 6472 | N | LEU | A | 824 | 15.157 | 65.043 | −9.788 | 1.00 | 15.04 | A |
| ATOM | 6473 | CA | LEU | A | 824 | 16.227 | 65.461 | −10.687 | 1.00 | 14.32 | A |
| ATOM | 6474 | CB | LEU | A | 824 | 15.976 | 66.908 | −11.132 | 1.00 | 14.06 | A |
| ATOM | 6475 | CG | LEU | A | 824 | 16.746 | 67.440 | −12.343 | 1.00 | 13.30 | A |
| ATOM | 6476 | CD1 | LEU | A | 824 | 15.995 | 68.633 | −12.939 | 1.00 | 13.69 | A |
| ATOM | 6477 | CD2 | LEU | A | 824 | 18.165 | 67.827 | −11.931 | 1.00 | 12.50 | A |
| ATOM | 6478 | C | LEU | A | 824 | 17.638 | 65.333 | −10.101 | 1.00 | 14.34 | A |
| ATOM | 6479 | O | LEU | A | 824 | 18.513 | 64.717 | −10.713 | 1.00 | 13.76 | A |
| ATOM | 6480 | N | GLN | A | 825 | 17.850 | 65.903 | −8.917 | 1.00 | 13.89 | A |
| ATOM | 6481 | CA | GLN | A | 825 | 19.164 | 65.876 | −8.273 | 1.00 | 14.21 | A |
| ATOM | 6482 | CB | GLN | A | 825 | 19.124 | 66.650 | −6.952 | 1.00 | 13.86 | A |
| ATOM | 6483 | CG | GLN | A | 825 | 18.152 | 66.091 | −5.922 | 1.00 | 14.09 | A |
| ATOM | 6484 | CD | GLN | A | 825 | 16.755 | 66.678 | −6.045 | 1.00 | 13.72 | A |
| ATOM | 6485 | OE1 | GLN | A | 825 | 16.340 | 67.096 | −7.123 | 1.00 | 14.70 | A |
| ATOM | 6486 | NE2 | GLN | A | 825 | 16.019 | 66.697 | −4.939 | 1.00 | 12.85 | A |
| ATOM | 6487 | C | GLN | A | 825 | 19.711 | 64.473 | −8.011 | 1.00 | 14.39 | A |
| ATOM | 6488 | O | GLN | A | 825 | 20.928 | 64.283 | −7.905 | 1.00 | 13.92 | A |
| ATOM | 6489 | N | ALA | A | 826 | 18.819 | 63.494 | −7.891 | 1.00 | 13.71 | A |
| ATOM | 6490 | CA | ALA | A | 826 | 19.241 | 62.120 | −7.640 | 1.00 | 13.79 | A |
| ATOM | 6491 | CB | ALA | A | 826 | 18.052 | 61.283 | −7.168 | 1.00 | 14.08 | A |
| ATOM | 6492 | C | ALA | A | 826 | 19.842 | 61.510 | −8.903 | 1.00 | 13.92 | A |
| ATOM | 6493 | O | ALA | A | 826 | 20.633 | 60.563 | −8.836 | 1.00 | 14.14 | A |
| ATOM | 6494 | N | ASN | A | 827 | 19.465 | 62.051 | −10.058 | 1.00 | 13.45 | A |
| ATOM | 6495 | CA | ASN | A | 827 | 19.974 | 61.541 | −11.321 | 1.00 | 12.85 | A |
| ATOM | 6496 | CB | ASN | A | 827 | 18.910 | 61.690 | −12.412 | 1.00 | 14.30 | A |
| ATOM | 6497 | CG | ASN | A | 827 | 17.765 | 60.704 | −12.234 | 1.00 | 15.77 | A |
| ATOM | 6498 | OD1 | ASN | A | 827 | 17.962 | 59.491 | −12.318 | 1.00 | 15.57 | A |
| ATOM | 6499 | ND2 | ASN | A | 827 | 16.567 | 61.218 | −11.972 | 1.00 | 16.77 | A |
| ATOM | 6500 | C | ASN | A | 827 | 21.290 | 62.200 | −11.726 | 1.00 | 12.33 | A |
| ATOM | 6501 | O | ASN | A | 827 | 21.813 | 61.956 | −12.812 | 1.00 | 11.17 | A |
| ATOM | 6502 | N | TYR | A | 828 | 21.820 | 63.046 | −10.846 | 1.00 | 12.25 | A |
| ATOM | 6503 | CA | TYR | A | 828 | 23.109 | 63.677 | −11.098 | 1.00 | 12.12 | A |
| ATOM | 6504 | CB | TYR | A | 828 | 23.200 | 65.057 | −10.438 | 1.00 | 12.01 | A |
| ATOM | 6505 | CG | TYR | A | 828 | 23.230 | 66.205 | −11.434 | 1.00 | 12.71 | A |
| ATOM | 6506 | CD1 | TYR | A | 828 | 22.160 | 66.432 | −12.297 | 1.00 | 11.49 | A |
| ATOM | 6507 | CE1 | TYR | A | 828 | 22.191 | 67.472 | −13.230 | 1.00 | 12.16 | A |
| ATOM | 6508 | CD2 | TYR | A | 828 | 24.338 | 67.051 | −11.522 | 1.00 | 11.99 | A |
| ATOM | 6509 | CE2 | TYR | A | 828 | 24.380 | 68.092 | −12.450 | 1.00 | 12.59 | A |
| ATOM | 6510 | CZ | TYR | A | 828 | 23.306 | 68.297 | −13.300 | 1.00 | 11.81 | A |
| ATOM | 6511 | OH | TYR | A | 828 | 23.356 | 69.311 | −14.231 | 1.00 | 12.90 | A |
| ATOM | 6512 | C | TYR | A | 828 | 24.151 | 62.747 | −10.485 | 1.00 | 11.84 | A |
| ATOM | 6513 | O | TYR | A | 828 | 23.953 | 62.220 | −9.387 | 1.00 | 11.86 | A |
| ATOM | 6514 | N | TYR | A | 829 | 25.246 | 62.540 | −11.208 | 1.00 | 11.25 | A |
| ATOM | 6515 | CA | TYR | A | 829 | 26.330 | 61.674 | −10.759 | 1.00 | 11.17 | A |
| ATOM | 6516 | CB | TYR | A | 829 | 26.365 | 60.376 | −11.576 | 1.00 | 11.21 | A |
| ATOM | 6517 | CG | TYR | A | 829 | 25.203 | 59.451 | −11.315 | 1.00 | 11.79 | A |
| ATOM | 6518 | CD1 | TYR | A | 829 | 24.042 | 59.516 | −12.087 | 1.00 | 11.45 | A |
| ATOM | 6519 | CE1 | TYR | A | 829 | 22.945 | 58.691 | −11.809 | 1.00 | 12.46 | A |
| ATOM | 6520 | CD2 | TYR | A | 829 | 25.248 | 58.540 | −10.261 | 1.00 | 11.44 | A |
| ATOM | 6521 | CE2 | TYR | A | 829 | 24.165 | 57.718 | −9.973 | 1.00 | 12.38 | A |
| ATOM | 6522 | CZ | TYR | A | 829 | 23.017 | 57.797 | −10.745 | 1.00 | 12.11 | A |
| ATOM | 6523 | OH | TYR | A | 829 | 21.944 | 56.993 | −10.433 | 1.00 | 12.60 | A |
| ATOM | 6524 | C | TYR | A | 829 | 27.676 | 62.365 | −10.916 | 1.00 | 10.97 | A |
| ATOM | 6525 | O | TYR | A | 829 | 27.799 | 63.357 | −11.630 | 1.00 | 10.60 | A |
| ATOM | 6526 | N | PRO | A | 830 | 28.708 | 61.846 | −10.239 | 1.00 | 10.67 | A |
| ATOM | 6527 | CD | PRO | A | 830 | 28.721 | 60.751 | −9.255 | 1.00 | 10.48 | A |
| ATOM | 6528 | CA | PRO | A | 830 | 30.030 | 62.464 | −10.363 | 1.00 | 10.76 | A |
| ATOM | 6529 | CB | PRO | A | 830 | 30.889 | 61.657 | −9.386 | 1.00 | 11.38 | A |
| ATOM | 6530 | CG | PRO | A | 830 | 29.906 | 61.114 | −8.396 | 1.00 | 11.76 | A |
| ATOM | 6531 | C | PRO | A | 830 | 30.534 | 62.284 | −11.796 | 1.00 | 10.70 | A |
| ATOM | 6532 | O | PRO | A | 830 | 30.317 | 61.230 | −12.400 | 1.00 | 10.23 | A |
| ATOM | 6533 | N | ILE | A | 831 | 31.179 | 63.309 | −12.348 | 1.00 | 10.28 | A |
| ATOM | 6534 | CA | ILE | A | 831 | 31.765 | 63.194 | −13.680 | 1.00 | 10.20 | A |
| ATOM | 6535 | CB | ILE | A | 831 | 31.182 | 64.207 | −14.704 | 1.00 | 10.32 | A |
| ATOM | 6536 | CG2 | ILE | A | 831 | 31.702 | 63.865 | −16.095 | 1.00 | 10.76 | A |
| ATOM | 6537 | CG1 | ILE | A | 831 | 29.648 | 64.174 | −14.698 | 1.00 | 10.68 | A |
| ATOM | 6538 | CD1 | ILE | A | 831 | 29.035 | 62.797 | −15.006 | 1.00 | 10.20 | A |
| ATOM | 6539 | C | ILE | A | 831 | 33.240 | 63.524 | −13.458 | 1.00 | 10.52 | A |
| ATOM | 6540 | O | ILE | A | 831 | 33.725 | 64.578 | −13.870 | 1.00 | 10.07 | A |
| ATOM | 6541 | N | PRO | A | 832 | 33.974 | 62.622 | −12.786 | 1.00 | 10.57 | A |
| ATOM | 6542 | CD | PRO | A | 832 | 33.527 | 61.378 | −12.135 | 1.00 | 11.09 | A |
| ATOM | 6543 | CA | PRO | A | 832 | 35.393 | 62.875 | −12.525 | 1.00 | 10.76 | A |
| ATOM | 6544 | CB | PRO | A | 832 | 35.807 | 61.697 | −11.632 | 1.00 | 10.28 | A |
| ATOM | 6545 | CG | PRO | A | 832 | 34.817 | 60.615 | −11.981 | 1.00 | 11.64 | A |
| ATOM | 6546 | C | PRO | A | 832 | 36.286 | 63.046 | −13.744 | 1.00 | 10.48 | A |
| ATOM | 6547 | O | PRO | A | 832 | 37.273 | 63.773 | −13.678 | 1.00 | 11.05 | A |
| ATOM | 6548 | N | SER | A | 833 | 35.954 | 62.395 | −14.856 | 1.00 | 10.53 | A |
| ATOM | 6549 | CA | SER | A | 833 | 36.783 | 62.546 | −16.049 | 1.00 | 10.75 | A |
| ATOM | 6550 | CB | SER | A | 833 | 37.996 | 61.609 | −15.981 | 1.00 | 11.44 | A |
| ATOM | 6551 | OG | SER | A | 833 | 37.643 | 60.273 | −16.285 | 1.00 | 13.55 | A |
| ATOM | 6552 | C | SER | A | 833 | 36.051 | 62.330 | −17.371 | 1.00 | 10.87 | A |
| ATOM | 6553 | O | SER | A | 833 | 36.581 | 62.672 | −18.425 | 1.00 | 10.60 | A |
| ATOM | 6554 | N | GLY | A | 834 | 34.845 | 61.769 | −17.327 | 1.00 | 10.57 | A |
| ATOM | 6555 | CA | GLY | A | 834 | 34.120 | 61.548 | −18.569 | 1.00 | 10.76 | A |
| ATOM | 6556 | C | GLY | A | 834 | 32.723 | 60.968 | −18.454 | 1.00 | 10.80 | A |
| ATOM | 6557 | O | GLY | A | 834 | 32.341 | 60.395 | −17.430 | 1.00 | 10.04 | A |
| ATOM | 6558 | N | MET | A | 835 | 31.959 | 61.116 | −19.532 | 1.00 | 11.25 | A |
| ATOM | 6559 | CA | MET | A | 835 | 30.591 | 60.621 | −19.588 | 1.00 | 11.48 | A |
| ATOM | 6560 | CB | MET | A | 835 | 29.642 | 61.643 | −18.952 | 1.00 | 12.22 | A |
| ATOM | 6561 | CG | MET | A | 835 | 29.507 | 62.945 | −19.744 | 1.00 | 11.77 | A |
| ATOM | 6562 | SD | MET | A | 835 | 28.685 | 64.277 | −18.832 | 1.00 | 13.73 | A |
| ATOM | 6563 | CE | MET | A | 835 | 27.082 | 63.543 | −18.510 | 1.00 | 12.36 | A |
| ATOM | 6564 | C | MET | A | 835 | 30.216 | 60.421 | −21.056 | 1.00 | 11.78 | A |
| ATOM | 6565 | O | MET | A | 835 | 30.779 | 61.071 | −21.945 | 1.00 | 11.10 | A |
| ATOM | 6566 | N | PHE | A | 836 | 29.277 | 59.519 | −21.318 | 1.00 | 11.62 | A |
| ATOM | 6567 | CA | PHE | A | 836 | 28.855 | 59.306 | −22.693 | 1.00 | 12.03 | A |
| ATOM | 6568 | CB | PHE | A | 836 | 29.915 | 58.499 | −23.481 | 1.00 | 11.91 | A |
| ATOM | 6569 | CG | PHE | A | 836 | 30.074 | 57.049 | −23.058 | 1.00 | 14.00 | A |
| ATOM | 6570 | CD1 | PHE | A | 836 | 29.200 | 56.066 | −23.525 | 1.00 | 14.04 | A |
| ATOM | 6571 | CD2 | PHE | A | 836 | 31.144 | 56.659 | −22.253 | 1.00 | 14.26 | A |
| ATOM | 6572 | CE1 | PHE | A | 836 | 29.392 | 54.715 | −23.202 | 1.00 | 16.21 | A |
| ATOM | 6573 | CE2 | PHE | A | 836 | 31.347 | 55.308 | −21.921 | 1.00 | 15.05 | A |
| ATOM | 6574 | CZ | PHE | A | 836 | 30.468 | 54.335 | −22.398 | 1.00 | 15.74 | A |
| ATOM | 6575 | C | PHE | A | 836 | 27.488 | 58.670 | −22.820 | 1.00 | 12.54 | A |
| ATOM | 6576 | O | PHE | A | 836 | 26.971 | 58.083 | −21.869 | 1.00 | 11.76 | A |
| ATOM | 6577 | N | ILE | A | 837 | 26.887 | 58.848 | −23.993 | 1.00 | 11.64 | A |
| ATOM | 6578 | CA | ILE | A | 837 | 25.593 | 58.260 | −24.303 | 1.00 | 12.13 | A |
| ATOM | 6579 | CB | ILE | A | 837 | 24.466 | 59.312 | −24.403 | 1.00 | 11.63 | A |
| ATOM | 6580 | CG2 | ILE | A | 837 | 24.032 | 59.742 | −23.007 | 1.00 | 11.57 | A |
| ATOM | 6581 | CG1 | ILE | A | 837 | 24.920 | 60.489 | −25.270 | 1.00 | 11.04 | A |
| ATOM | 6582 | CD1 | ILE | A | 837 | 23.771 | 61.408 | −25.698 | 1.00 | 10.76 | A |
| ATOM | 6583 | C | ILE | A | 837 | 25.758 | 57.599 | −25.660 | 1.00 | 12.23 | A |
| ATOM | 6584 | O | ILE | A | 837 | 26.612 | 58.006 | −26.457 | 1.00 | 11.97 | A |
| ATOM | 6585 | N | GLU | A | 838 | 24.955 | 56.579 | −25.928 | 1.00 | 13.26 | A |
| ATOM | 6586 | CA | GLU | A | 838 | 25.063 | 55.887 | −27.203 | 1.00 | 13.21 | A |
| ATOM | 6587 | CB | GLU | A | 838 | 26.232 | 54.901 | −27.153 | 1.00 | 14.34 | A |
| ATOM | 6588 | CG | GLU | A | 838 | 25.978 | 53.751 | −26.166 | 1.00 | 15.15 | A |
| ATOM | 6589 | CD | GLU | A | 838 | 27.091 | 52.715 | −26.120 | 1.00 | 16.72 | A |
| ATOM | 6590 | OE1 | GLU | A | 838 | 26.937 | 51.720 | −25.382 | 1.00 | 18.45 | A |
| ATOM | 6591 | OE2 | GLU | A | 838 | 28.114 | 52.884 | −26.812 | 1.00 | 17.74 | A |
| ATOM | 6592 | C | GLU | A | 838 | 23.807 | 55.100 | −27.538 | 1.00 | 14.05 | A |
| ATOM | 6593 | O | GLU | A | 838 | 22.977 | 54.827 | −26.671 | 1.00 | 13.44 | A |
| ATOM | 6594 | N | ASP | A | 839 | 23.655 | 54.773 | −28.816 | 1.00 | 14.77 | A |
| ATOM | 6595 | CA | ASP | A | 839 | 22.561 | 53.913 | −29.240 | 1.00 | 15.54 | A |
| ATOM | 6596 | CB | ASP | A | 839 | 21.494 | 54.633 | −30.085 | 1.00 | 15.80 | A |
| ATOM | 6597 | CG | ASP | A | 839 | 22.061 | 55.405 | −31.260 | 1.00 | 15.71 | A |
| ATOM | 6598 | OD1 | ASP | A | 839 | 23.091 | 55.001 | −31.838 | 1.00 | 15.80 | A |
| ATOM | 6599 | OD2 | ASP | A | 839 | 21.434 | 56.424 | −31.618 | 1.00 | 17.81 | A |
| ATOM | 6600 | C | ASP | A | 839 | 23.267 | 52.823 | −30.028 | 1.00 | 16.22 | A |
| ATOM | 6601 | O | ASP | A | 839 | 24.471 | 52.624 | −29.859 | 1.00 | 15.94 | A |
| ATOM | 6602 | N | ALA | A | 840 | 22.545 | 52.114 | −30.882 | 1.00 | 16.73 | A |
| ATOM | 6603 | CA | ALA | A | 840 | 23.160 | 51.042 | −31.645 | 1.00 | 17.26 | A |
| ATOM | 6604 | CB | ALA | A | 840 | 22.083 | 50.279 | −32.418 | 1.00 | 18.36 | A |
| ATOM | 6605 | C | ALA | A | 840 | 24.258 | 51.500 | −32.603 | 1.00 | 17.43 | A |
| ATOM | 6606 | O | ALA | A | 840 | 25.200 | 50.749 | −32.871 | 1.00 | 17.52 | A |
| ATOM | 6607 | N | ASN | A | 841 | 24.164 | 52.733 | −33.096 | 1.00 | 16.94 | A |
| ATOM | 6608 | CA | ASN | A | 841 | 25.136 | 53.215 | −34.076 | 1.00 | 17.30 | A |
| ATOM | 6609 | CB | ASN | A | 841 | 24.406 | 53.557 | −35.377 | 1.00 | 18.50 | A |
| ATOM | 6610 | CG | ASN | A | 841 | 23.592 | 52.402 | −35.900 | 1.00 | 20.17 | A |
| ATOM | 6611 | OD1 | ASN | A | 841 | 24.122 | 51.317 | −36.126 | 1.00 | 21.04 | A |
| ATOM | 6612 | ND2 | ASN | A | 841 | 22.295 | 52.625 | −36.092 | 1.00 | 19.98 | A |
| ATOM | 6613 | C | ASN | A | 841 | 26.021 | 54.402 | −33.725 | 1.00 | 16.82 | A |
| ATOM | 6614 | O | ASN | A | 841 | 27.129 | 54.520 | −34.254 | 1.00 | 16.18 | A |
| ATOM | 6615 | N | THR | A | 842 | 25.542 | 55.278 | −32.850 | 1.00 | 16.36 | A |
| ATOM | 6616 | CA | THR | A | 842 | 26.298 | 56.479 | −32.517 | 1.00 | 16.16 | A |
| ATOM | 6617 | CB | THR | A | 842 | 25.564 | 57.724 | −33.075 | 1.00 | 16.66 | A |
| ATOM | 6618 | OG1 | THR | A | 842 | 25.204 | 57.490 | −34.444 | 1.00 | 17.07 | A |
| ATOM | 6619 | CG2 | THR | A | 842 | 26.452 | 58.958 | −32.999 | 1.00 | 16.18 | A |
| ATOM | 6620 | C | THR | A | 842 | 26.547 | 56.699 | −31.031 | 1.00 | 15.76 | A |
| ATOM | 6621 | O | THR | A | 842 | 25.713 | 56.369 | −30.190 | 1.00 | 16.36 | A |
| ATOM | 6622 | N | ARG | A | 843 | 27.704 | 57.271 | −30.718 | 1.00 | 14.94 | A |
| ATOM | 6623 | CA | ARG | A | 843 | 28.057 | 57.573 | −29.335 | 1.00 | 13.57 | A |
| ATOM | 6624 | CB | ARG | A | 843 | 29.056 | 56.557 | −28.775 | 1.00 | 13.32 | A |
| ATOM | 6625 | CG | ARG | A | 843 | 29.577 | 56.928 | −27.375 | 1.00 | 12.73 | A |
| ATOM | 6626 | CD | ARG | A | 843 | 30.764 | 56.075 | −26.924 | 1.00 | 12.83 | A |
| ATOM | 6627 | NE | ARG | A | 843 | 30.395 | 54.696 | −26.606 | 1.00 | 12.99 | A |
| ATOM | 6628 | CZ | ARG | A | 843 | 31.216 | 53.820 | −26.029 | 1.00 | 14.10 | A |
| ATOM | 6629 | NH1 | ARG | A | 843 | 32.452 | 54.180 | −25.705 | 1.00 | 13.39 | A |
| ATOM | 6630 | NH2 | ARG | A | 843 | 30.809 | 52.581 | −25.772 | 1.00 | 13.79 | A |
| ATOM | 6631 | C | ARG | A | 843 | 28.685 | 58.954 | −29.266 | 1.00 | 12.96 | A |
| ATOM | 6632 | O | ARG | A | 843 | 29.402 | 59.368 | −30.177 | 1.00 | 12.57 | A |
| ATOM | 6633 | N | LEU | A | 844 | 28.396 | 59.666 | −28.185 | 1.00 | 12.37 | A |
| ATOM | 6634 | CA | LEU | A | 844 | 28.969 | 60.985 | −27.967 | 1.00 | 12.14 | A |
| ATOM | 6635 | CB | LEU | A | 844 | 27.890 | 62.072 | −27.967 | 1.00 | 11.38 | A |
| ATOM | 6636 | CG | LEU | A | 844 | 28.458 | 63.488 | −27.823 | 1.00 | 11.36 | A |
| ATOM | 6637 | CD1 | LEU | A | 844 | 29.375 | 63.778 | −29.005 | 1.00 | 11.45 | A |
| ATOM | 6638 | CD2 | LEU | A | 844 | 27.334 | 64.513 | −27.757 | 1.00 | 11.60 | A |
| ATOM | 6639 | C | LEU | A | 844 | 29.624 | 60.922 | −26.598 | 1.00 | 11.63 | A |
| ATOM | 6640 | O | LEU | A | 844 | 28.958 | 60.622 | −25.601 | 1.00 | 11.42 | A |
| ATOM | 6641 | N | THR | A | 845 | 30.926 | 61.187 | −26.553 | 1.00 | 11.41 | A |
| ATOM | 6642 | CA | THR | A | 845 | 31.662 | 61.150 | −25.295 | 1.00 | 11.95 | A |
| ATOM | 6643 | CB | THR | A | 845 | 32.833 | 60.144 | −25.357 | 1.00 | 12.35 | A |
| ATOM | 6644 | OG1 | THR | A | 845 | 32.335 | 58.849 | −25.721 | 1.00 | 13.45 | A |
| ATOM | 6645 | CG2 | THR | A | 845 | 33.537 | 60.051 | −24.003 | 1.00 | 12.11 | A |
| ATOM | 6646 | C | THR | A | 845 | 32.244 | 62.520 | −24.965 | 1.00 | 11.55 | A |
| ATOM | 6647 | O | THR | A | 845 | 32.867 | 63.162 | −25.808 | 1.00 | 11.72 | A |
| ATOM | 6648 | N | LEU | A | 846 | 32.037 | 62.962 | −23.732 | 1.00 | 11.76 | A |
| ATOM | 6649 | CA | LEU | A | 846 | 32.572 | 64.240 | −23.290 | 1.00 | 11.18 | A |
| ATOM | 6650 | CB | LEU | A | 846 | 31.454 | 65.127 | −22.730 | 1.00 | 10.76 | A |
| ATOM | 6651 | CG | LEU | A | 846 | 31.881 | 66.481 | −22.148 | 1.00 | 12.31 | A |
| ATOM | 6652 | CD1 | LEU | A | 846 | 32.435 | 67.361 | −23.263 | 1.00 | 12.08 | A |
| ATOM | 6653 | CD2 | LEU | A | 846 | 30.693 | 67.164 | −21.471 | 1.00 | 12.32 | A |
| ATOM | 6654 | C | LEU | A | 846 | 33.584 | 63.924 | −22.196 | 1.00 | 10.28 | A |
| ATOM | 6655 | O | LEU | A | 846 | 33.226 | 63.361 | −21.161 | 1.00 | 9.21 | A |
| ATOM | 6656 | N | LEU | A | 847 | 34.847 | 64.261 | −22.443 | 1.00 | 10.60 | A |
| ATOM | 6657 | CA | LEU | A | 847 | 35.917 | 64.023 | −21.474 | 1.00 | 10.37 | A |
| ATOM | 6658 | CB | LEU | A | 847 | 37.187 | 63.511 | −22.175 | 1.00 | 10.15 | A |
| ATOM | 6659 | CG | LEU | A | 847 | 37.166 | 62.164 | −22.922 | 1.00 | 9.45 | A |
| ATOM | 6660 | CD1 | LEU | A | 847 | 36.463 | 61.116 | −22.070 | 1.00 | 8.42 | A |
| ATOM | 6661 | CD2 | LEU | A | 847 | 36.465 | 62.318 | −24.268 | 1.00 | 9.43 | A |
| ATOM | 6662 | C | LEU | A | 847 | 36.213 | 65.350 | −20.774 | 1.00 | 11.19 | A |
| ATOM | 6663 | O | LEU | A | 847 | 36.141 | 66.412 | −21.401 | 1.00 | 10.50 | A |
| ATOM | 6664 | N | THR | A | 848 | 36.557 | 65.291 | −19.487 | 1.00 | 9.84 | A |
| ATOM | 6665 | CA | THR | A | 848 | 36.826 | 66.507 | −18.722 | 1.00 | 10.43 | A |
| ATOM | 6666 | CB | THR | A | 848 | 35.864 | 66.642 | −17.535 | 1.00 | 11.02 | A |
| ATOM | 6667 | OG1 | THR | A | 848 | 36.281 | 65.757 | −16.485 | 1.00 | 12.33 | A |
| ATOM | 6668 | CG2 | THR | A | 848 | 34.448 | 66.292 | −17.953 | 1.00 | 11.23 | A |
| ATOM | 6669 | C | THR | A | 848 | 38.233 | 66.590 | −18.147 | 1.00 | 10.77 | A |
| ATOM | 6670 | O | THR | A | 848 | 38.909 | 65.574 | −17.958 | 1.00 | 10.97 | A |
| ATOM | 6671 | N | GLY | A | 849 | 38.659 | 67.820 | −17.869 | 1.00 | 10.37 | A |
| ATOM | 6672 | CA | GLY | A | 849 | 39.968 | 68.042 | −17.289 | 1.00 | 10.72 | A |
| ATOM | 6673 | C | GLY | A | 849 | 39.792 | 68.412 | −15.828 | 1.00 | 10.76 | A |
| ATOM | 6674 | O | GLY | A | 849 | 40.719 | 68.881 | −15.167 | 1.00 | 10.54 | A |
| ATOM | 6675 | N | GLN | A | 850 | 38.580 | 68.198 | −15.328 | 1.00 | 10.62 | A |
| ATOM | 6676 | CA | GLN | A | 850 | 38.250 | 68.497 | −13.942 | 1.00 | 10.79 | A |
| ATOM | 6677 | CB | GLN | A | 850 | 38.049 | 70.008 | −13.758 | 1.00 | 10.45 | A |
| ATOM | 6678 | CG | GLN | A | 850 | 36.893 | 70.617 | −14.578 | 1.00 | 10.58 | A |
| ATOM | 6679 | CD | GLN | A | 850 | 37.197 | 70.722 | −16.060 | 1.00 | 11.77 | A |
| ATOM | 6680 | OE1 | GLN | A | 850 | 38.292 | 71.135 | −16.453 | 1.00 | 12.48 | A |
| ATOM | 6681 | NE2 | GLN | A | 850 | 36.220 | 70.365 | −16.898 | 1.00 | 10.17 | A |
| ATOM | 6682 | C | GLN | A | 850 | 36.969 | 67.764 | −13.552 | 1.00 | 11.10 | A |
| ATOM | 6683 | O | GLN | A | 850 | 36.101 | 67.530 | −14.394 | 1.00 | 10.49 | A |
| ATOM | 6684 | N | PRO | A | 851 | 36.838 | 67.380 | −12.270 | 1.00 | 10.84 | A |
| ATOM | 6685 | CD | PRO | A | 851 | 37.835 | 67.430 | −11.185 | 1.00 | 10.05 | A |
| ATOM | 6686 | CA | PRO | A | 851 | 35.624 | 66.678 | −11.839 | 1.00 | 10.83 | A |
| ATOM | 6687 | CB | PRO | A | 851 | 36.051 | 66.010 | −10.534 | 1.00 | 10.06 | A |
| ATOM | 6688 | CG | PRO | A | 851 | 37.021 | 67.017 | −9.960 | 1.00 | 10.19 | A |
| ATOM | 6689 | C | PRO | A | 851 | 34.484 | 67.673 | −11.637 | 1.00 | 11.55 | A |
| ATOM | 6690 | O | PRO | A | 851 | 34.668 | 68.732 | −11.021 | 1.00 | 10.88 | A |
| ATOM | 6691 | N | LEU | A | 852 | 33.316 | 67.327 | −12.171 | 1.00 | 11.38 | A |
| ATOM | 6692 | CA | LEU | A | 852 | 32.122 | 68.164 | −12.075 | 1.00 | 11.58 | A |
| ATOM | 6693 | CB | LEU | A | 852 | 32.015 | 69.075 | −13.305 | 1.00 | 11.68 | A |
| ATOM | 6694 | CG | LEU | A | 852 | 33.136 | 70.099 | −13.523 | 1.00 | 11.56 | A |
| ATOM | 6695 | CD1 | LEU | A | 852 | 33.028 | 70.713 | −14.916 | 1.00 | 12.15 | A |
| ATOM | 6696 | CD2 | LEU | A | 852 | 33.038 | 71.184 | −12.458 | 1.00 | 11.06 | A |
| ATOM | 6697 | C | LEU | A | 852 | 30.902 | 67.247 | −12.012 | 1.00 | 11.91 | A |
| ATOM | 6698 | O | LEU | A | 852 | 31.016 | 66.044 | −12.247 | 1.00 | 12.75 | A |
| ATOM | 6699 | N | GLY | A | 853 | 29.739 | 67.807 | −11.694 | 1.00 | 11.21 | A |
| ATOM | 6700 | CA | GLY | A | 853 | 28.537 | 66.994 | −11.624 | 1.00 | 11.60 | A |
| ATOM | 6701 | C | GLY | A | 853 | 27.792 | 66.992 | −12.949 | 1.00 | 11.73 | A |
| ATOM | 6702 | O | GLY | A | 853 | 27.819 | 67.980 | −13.682 | 1.00 | 11.22 | A |
| ATOM | 6703 | N | GLY | A | 854 | 27.116 | 65.891 | −13.265 | 1.00 | 11.53 | A |
| ATOM | 6704 | CA | GLY | A | 854 | 26.398 | 65.840 | −14.528 | 1.00 | 12.12 | A |
| ATOM | 6705 | C | GLY | A | 854 | 25.351 | 64.753 | −14.623 | 1.00 | 12.05 | A |
| ATOM | 6706 | O | GLY | A | 854 | 25.157 | 63.980 | −13.684 | 1.00 | 11.99 | A |
| ATOM | 6707 | N | SER | A | 855 | 24.680 | 64.686 | −15.770 | 1.00 | 12.94 | A |
| ATOM | 6708 | CA | SER | A | 855 | 23.634 | 63.689 | −15.976 | 1.00 | 13.86 | A |
| ATOM | 6709 | CB | SER | A | 855 | 22.441 | 64.003 | −15.065 | 1.00 | 14.27 | A |
| ATOM | 6710 | OG | SER | A | 855 | 21.402 | 63.043 | −15.195 | 1.00 | 14.40 | A |
| ATOM | 6711 | C | SER | A | 855 | 23.162 | 63.680 | −17.423 | 1.00 | 14.27 | A |
| ATOM | 6712 | O | SER | A | 855 | 23.716 | 64.374 | −18.279 | 1.00 | 13.52 | A |
| ATOM | 6713 | N | SER | A | 856 | 22.143 | 62.863 | −17.675 | 1.00 | 14.72 | A |
| ATOM | 6714 | CA | SER | A | 856 | 21.492 | 62.752 | −18.978 | 1.00 | 14.69 | A |
| ATOM | 6715 | CB | SER | A | 856 | 21.838 | 61.431 | −19.665 | 1.00 | 15.13 | A |
| ATOM | 6716 | OG | SER | A | 856 | 21.175 | 61.330 | −20.915 | 1.00 | 15.73 | A |
| ATOM | 6717 | C | SER | A | 856 | 20.025 | 62.750 | −18.560 | 1.00 | 15.42 | A |
| ATOM | 6718 | O | SER | A | 856 | 19.469 | 61.702 | −18.216 | 1.00 | 14.82 | A |
| ATOM | 6719 | N | LEU | A | 857 | 19.404 | 63.926 | −18.570 | 1.00 | 14.83 | A |
| ATOM | 6720 | CA | LEU | A | 857 | 18.023 | 64.041 | −18.121 | 1.00 | 15.04 | A |
| ATOM | 6721 | CB | LEU | A | 857 | 17.787 | 65.444 | −17.555 | 1.00 | 15.79 | A |
| ATOM | 6722 | CG | LEU | A | 857 | 18.656 | 65.766 | −16.335 | 1.00 | 15.73 | A |
| ATOM | 6723 | CD1 | LEU | A | 857 | 18.423 | 67.199 | −15.903 | 1.00 | 16.46 | A |
| ATOM | 6724 | CD2 | LEU | A | 857 | 18.325 | 64.802 | −15.193 | 1.00 | 16.82 | A |
| ATOM | 6725 | C | LEU | A | 857 | 16.959 | 63.698 | −19.153 | 1.00 | 15.01 | A |
| ATOM | 6726 | O | LEU | A | 857 | 15.774 | 63.648 | −18.831 | 1.00 | 16.21 | A |
| ATOM | 6727 | N | ALA | A | 858 | 17.386 | 63.455 | −20.384 | 1.00 | 14.17 | A |
| ATOM | 6728 | CA | ALA | A | 858 | 16.476 | 63.095 | −21.463 | 1.00 | 15.27 | A |
| ATOM | 6729 | CB | ALA | A | 858 | 15.840 | 64.353 | −22.080 | 1.00 | 14.55 | A |
| ATOM | 6730 | C | ALA | A | 858 | 17.287 | 62.348 | −22.508 | 1.00 | 15.22 | A |
| ATOM | 6731 | O | ALA | A | 858 | 18.491 | 62.561 | −22.631 | 1.00 | 15.05 | A |
| ATOM | 6732 | N | SER | A | 859 | 16.628 | 61.461 | −23.247 | 1.00 | 15.43 | A |
| ATOM | 6733 | CA | SER | A | 859 | 17.300 | 60.690 | −24.280 | 1.00 | 15.40 | A |
| ATOM | 6734 | CB | SER | A | 859 | 16.272 | 59.883 | −25.085 | 1.00 | 15.64 | A |
| ATOM | 6735 | OG | SER | A | 859 | 16.898 | 59.143 | −26.118 | 1.00 | 15.79 | A |
| ATOM | 6736 | C | SER | A | 859 | 18.067 | 61.626 | −25.208 | 1.00 | 15.90 | A |
| ATOM | 6737 | O | SER | A | 859 | 17.551 | 62.673 | −25.612 | 1.00 | 16.40 | A |
| ATOM | 6738 | N | GLY | A | 860 | 19.300 | 61.249 | −25.531 | 1.00 | 15.03 | A |
| ATOM | 6739 | CA | GLY | A | 860 | 20.128 | 62.051 | −26.416 | 1.00 | 14.88 | A |
| ATOM | 6740 | C | GLY | A | 860 | 20.837 | 63.235 | −25.778 | 1.00 | 14.74 | A |
| ATOM | 6741 | O | GLY | A | 860 | 21.556 | 63.971 | −26.461 | 1.00 | 14.83 | A |
| ATOM | 6742 | N | GLU | A | 861 | 20.657 | 63.427 | −24.477 | 1.00 | 14.40 | A |
| ATOM | 6743 | CA | GLU | A | 861 | 21.296 | 64.553 | −23.802 | 1.00 | 14.66 | A |
| ATOM | 6744 | CB | GLU | A | 861 | 20.274 | 65.343 | −22.982 | 1.00 | 15.09 | A |
| ATOM | 6745 | CG | GLU | A | 861 | 19.167 | 66.015 | −23.771 | 1.00 | 15.73 | A |
| ATOM | 6746 | CD | GLU | A | 861 | 18.274 | 66.866 | −22.881 | 1.00 | 16.26 | A |
| ATOM | 6747 | OE1 | GLU | A | 861 | 18.499 | 66.896 | −21.648 | 1.00 | 16.77 | A |
| ATOM | 6748 | OE2 | GLU | A | 861 | 17.349 | 67.507 | −23.410 | 1.00 | 16.56 | A |
| ATOM | 6749 | C | GLU | A | 861 | 22.441 | 64.196 | −22.864 | 1.00 | 14.50 | A |
| ATOM | 6750 | O | GLU | A | 861 | 22.521 | 63.086 | −22.338 | 1.00 | 13.85 | A |
| ATOM | 6751 | N | LEU | A | 862 | 23.321 | 65.171 | −22.668 | 1.00 | 14.08 | A |
| ATOM | 6752 | CA | LEU | A | 862 | 24.443 | 65.069 | −21.740 | 1.00 | 14.26 | A |
| ATOM | 6753 | CB | LEU | A | 862 | 25.767 | 64.772 | −22.452 | 1.00 | 13.73 | A |
| ATOM | 6754 | CG | LEU | A | 862 | 26.100 | 63.391 | −23.028 | 1.00 | 13.69 | A |
| ATOM | 6755 | CD1 | LEU | A | 862 | 27.463 | 63.459 | −23.706 | 1.00 | 12.07 | A |
| ATOM | 6756 | CD2 | LEU | A | 862 | 26.113 | 62.342 | −21.933 | 1.00 | 13.40 | A |
| ATOM | 6757 | C | LEU | A | 862 | 24.516 | 66.467 | −21.156 | 1.00 | 14.16 | A |
| ATOM | 6758 | O | LEU | A | 862 | 24.308 | 67.449 | −21.870 | 1.00 | 14.09 | A |
| ATOM | 6759 | N | GLU | A | 863 | 24.773 | 66.574 | −19.862 | 1.00 | 13.64 | A |
| ATOM | 6760 | CA | GLU | A | 863 | 24.902 | 67.893 | −19.272 | 1.00 | 13.16 | A |
| ATOM | 6761 | CB | GLU | A | 863 | 23.535 | 68.453 | −18.860 | 1.00 | 13.14 | A |
| ATOM | 6762 | CG | GLU | A | 863 | 23.007 | 68.027 | −17.508 | 1.00 | 12.61 | A |
| ATOM | 6763 | CD | GLU | A | 863 | 21.683 | 68.701 | −17.191 | 1.00 | 13.43 | A |
| ATOM | 6764 | OE1 | GLU | A | 863 | 21.529 | 69.210 | −16.061 | 1.00 | 14.53 | A |
| ATOM | 6765 | OE2 | GLU | A | 863 | 20.792 | 68.719 | −18.071 | 1.00 | 14.31 | A |
| ATOM | 6766 | C | GLU | A | 863 | 25.851 | 67.819 | −18.094 | 1.00 | 12.83 | A |
| ATOM | 6767 | O | GLU | A | 863 | 25.918 | 66.807 | −17.394 | 1.00 | 11.84 | A |
| ATOM | 6768 | N | ILE | A | 864 | 26.600 | 68.894 | −17.891 | 1.00 | 12.45 | A |
| ATOM | 6769 | CA | ILE | A | 864 | 27.574 | 68.942 | −16.814 | 1.00 | 12.34 | A |
| ATOM | 6770 | CB | ILE | A | 864 | 28.953 | 68.477 | −17.355 | 1.00 | 13.29 | A |
| ATOM | 6771 | CG2 | ILE | A | 864 | 29.406 | 69.390 | −18.497 | 1.00 | 13.38 | A |
| ATOM | 6772 | CG1 | ILE | A | 864 | 29.979 | 68.411 | −16.226 | 1.00 | 12.87 | A |
| ATOM | 6773 | CD1 | ILE | A | 864 | 31.255 | 67.696 | −16.632 | 1.00 | 15.03 | A |
| ATOM | 6774 | C | ILE | A | 864 | 27.626 | 70.363 | −16.245 | 1.00 | 12.28 | A |
| ATOM | 6775 | O | ILE | A | 864 | 27.749 | 71.339 | −16.989 | 1.00 | 12.71 | A |
| ATOM | 6776 | N | MET | A | 865 | 27.506 | 70.466 | −14.925 | 1.00 | 12.05 | A |
| ATOM | 6777 | CA | MET | A | 865 | 27.504 | 71.751 | −14.229 | 1.00 | 12.26 | A |
| ATOM | 6778 | CB | MET | A | 865 | 27.077 | 71.548 | −12.773 | 1.00 | 12.43 | A |
| ATOM | 6779 | CG | MET | A | 865 | 26.485 | 72.787 | −12.127 | 1.00 | 11.69 | A |
| ATOM | 6780 | SD | MET | A | 865 | 24.884 | 73.169 | −12.872 | 1.00 | 13.86 | A |
| ATOM | 6781 | CE | MET | A | 865 | 23.850 | 71.943 | −12.046 | 1.00 | 12.21 | A |
| ATOM | 6782 | C | MET | A | 865 | 28.872 | 72.436 | −14.273 | 1.00 | 12.23 | A |
| ATOM | 6783 | O | MET | A | 865 | 29.903 | 71.792 | −14.082 | 1.00 | 11.54 | A |
| ATOM | 6784 | N | GLN | A | 866 | 28.869 | 73.748 | −14.501 | 1.00 | 12.20 | A |
| ATOM | 6785 | CA | GLN | A | 866 | 30.106 | 74.520 | −14.592 | 1.00 | 12.17 | A |
| ATOM | 6786 | CB | GLN | A | 866 | 29.973 | 75.568 | −15.694 | 1.00 | 12.07 | A |
| ATOM | 6787 | CG | GLN | A | 866 | 29.638 | 74.959 | −17.037 | 1.00 | 13.16 | A |
| ATOM | 6788 | CD | GLN | A | 866 | 30.648 | 73.908 | −17.455 | 1.00 | 13.74 | A |
| ATOM | 6789 | OE1 | GLN | A | 866 | 31.806 | 74.221 | −17.750 | 1.00 | 13.62 | A |
| ATOM | 6790 | NE2 | GLN | A | 866 | 30.219 | 72.648 | −17.468 | 1.00 | 12.85 | A |
| ATOM | 6791 | C | GLN | A | 866 | 30.501 | 75.186 | −13.279 | 1.00 | 12.32 | A |
| ATOM | 6792 | O | GLN | A | 866 | 31.672 | 75.190 | −12.910 | 1.00 | 12.03 | A |
| ATOM | 6793 | N | ASP | A | 867 | 29.523 | 75.772 | −12.595 | 1.00 | 12.35 | A |
| ATOM | 6794 | CA | ASP | A | 867 | 29.759 | 76.405 | −11.303 | 1.00 | 12.58 | A |
| ATOM | 6795 | CB | ASP | A | 867 | 30.532 | 77.722 | −11.450 | 1.00 | 12.46 | A |
| ATOM | 6796 | CG | ASP | A | 867 | 31.204 | 78.156 | −10.147 | 1.00 | 12.23 | A |
| ATOM | 6797 | OD1 | ASP | A | 867 | 31.032 | 77.471 | −9.115 | 1.00 | 11.85 | A |
| ATOM | 6798 | OD2 | ASP | A | 867 | 31.912 | 79.182 | −10.157 | 1.00 | 12.76 | A |
| ATOM | 6799 | C | ASP | A | 867 | 28.409 | 76.665 | −10.664 | 1.00 | 13.58 | A |
| ATOM | 6800 | O | ASP | A | 867 | 27.371 | 76.624 | −11.334 | 1.00 | 12.90 | A |
| ATOM | 6801 | N | ARG | A | 868 | 28.427 | 76.923 | −9.363 | 1.00 | 13.40 | A |
| ATOM | 6802 | CA | ARG | A | 868 | 27.210 | 77.183 | −8.618 | 1.00 | 14.19 | A |
| ATOM | 6803 | CB | ARG | A | 868 | 26.683 | 75.872 | −8.023 | 1.00 | 13.52 | A |
| ATOM | 6804 | CG | ARG | A | 868 | 27.697 | 75.114 | −7.172 | 1.00 | 13.57 | A |
| ATOM | 6805 | CD | ARG | A | 868 | 27.390 | 73.611 | −7.182 | 1.00 | 13.30 | A |
| ATOM | 6806 | NE | ARG | A | 868 | 26.035 | 73.331 | −6.723 | 1.00 | 12.07 | A |
| ATOM | 6807 | CZ | ARG | A | 868 | 25.714 | 73.036 | −5.467 | 1.00 | 12.96 | A |
| ATOM | 6808 | NH1 | ARG | A | 868 | 26.655 | 72.970 | −4.534 | 1.00 | 11.71 | A |
| ATOM | 6809 | NH2 | ARG | A | 868 | 24.445 | 72.822 | −5.141 | 1.00 | 11.83 | A |
| ATOM | 6810 | C | ARG | A | 868 | 27.513 | 78.189 | −7.519 | 1.00 | 14.32 | A |
| ATOM | 6811 | O | ARG | A | 868 | 28.536 | 78.087 | −6.840 | 1.00 | 14.96 | A |
| ATOM | 6812 | N | ARG | A | 869 | 26.627 | 79.167 | −7.363 | 1.00 | 14.46 | A |
| ATOM | 6813 | CA | ARG | A | 869 | 26.777 | 80.213 | −6.352 | 1.00 | 14.99 | A |
| ATOM | 6814 | CB | ARG | A | 869 | 26.938 | 81.578 | −7.037 | 1.00 | 15.61 | A |
| ATOM | 6815 | CG | ARG | A | 869 | 27.263 | 82.745 | −6.104 | 1.00 | 16.25 | A |
| ATOM | 6816 | CD | ARG | A | 869 | 27.492 | 84.039 | −6.890 | 1.00 | 18.29 | A |
| ATOM | 6817 | NE | ARG | A | 869 | 27.678 | 85.202 | −6.019 | 1.00 | 19.10 | A |
| ATOM | 6818 | CZ | ARG | A | 869 | 28.805 | 85.495 | −5.376 | 1.00 | 19.20 | A |
| ATOM | 6819 | NH1 | ARG | A | 869 | 29.870 | 84.718 | −5.500 | 1.00 | 19.53 | A |
| ATOM | 6820 | NH2 | ARG | A | 869 | 28.865 | 86.569 | −4.597 | 1.00 | 19.46 | A |
| ATOM | 6821 | C | ARG | A | 869 | 25.497 | 80.164 | −5.521 | 1.00 | 15.62 | A |
| ATOM | 6822 | O | ARG | A | 869 | 24.416 | 80.479 | −6.015 | 1.00 | 15.11 | A |
| ATOM | 6823 | N | LEU | A | 870 | 25.629 | 79.749 | −4.265 | 1.00 | 16.14 | A |
| ATOM | 6824 | CA | LEU | A | 870 | 24.490 | 79.609 | −3.362 | 1.00 | 17.85 | A |
| ATOM | 6825 | CB | LEU | A | 870 | 24.396 | 78.153 | −2.915 | 1.00 | 18.19 | A |
| ATOM | 6826 | CG | LEU | A | 870 | 24.411 | 77.209 | −4.122 | 1.00 | 19.60 | A |
| ATOM | 6827 | CD1 | LEU | A | 870 | 24.773 | 75.815 | −3.688 | 1.00 | 20.54 | A |
| ATOM | 6828 | CD2 | LEU | A | 870 | 23.056 | 77.244 | −4.818 | 1.00 | 20.32 | A |
| ATOM | 6829 | C | LEU | A | 870 | 24.583 | 80.530 | −2.148 | 1.00 | 18.52 | A |
| ATOM | 6830 | O | LEU | A | 870 | 25.576 | 80.523 | −1.419 | 1.00 | 18.31 | A |
| ATOM | 6831 | N | ALA | A | 871 | 23.529 | 81.308 | −1.926 | 1.00 | 19.42 | A |
| ATOM | 6832 | CA | ALA | A | 871 | 23.496 | 82.259 | −0.823 | 1.00 | 21.24 | A |
| ATOM | 6833 | CB | ALA | A | 871 | 22.452 | 83.341 | −1.109 | 1.00 | 20.93 | A |
| ATOM | 6834 | C | ALA | A | 871 | 23.244 | 81.658 | 0.556 | 1.00 | 22.09 | A |
| ATOM | 6835 | O | ALA | A | 871 | 23.668 | 82.223 | 1.563 | 1.00 | 23.73 | A |
| ATOM | 6836 | N | SER | A | 872 | 22.567 | 80.518 | 0.617 | 1.00 | 21.91 | A |
| ATOM | 6837 | CA | SER | A | 872 | 22.272 | 79.922 | 1.914 | 1.00 | 21.65 | A |
| ATOM | 6838 | CB | SER | A | 872 | 20.769 | 79.658 | 2.034 | 1.00 | 23.19 | A |
| ATOM | 6839 | OG | SER | A | 872 | 20.338 | 78.730 | 1.055 | 1.00 | 26.58 | A |
| ATOM | 6840 | C | SER | A | 872 | 23.036 | 78.646 | 2.239 | 1.00 | 20.75 | A |
| ATOM | 6841 | O | SER | A | 872 | 23.617 | 78.005 | 1.364 | 1.00 | 19.55 | A |
| ATOM | 6842 | N | ASP | A | 873 | 23.031 | 78.301 | 3.522 | 1.00 | 19.61 | A |
| ATOM | 6843 | CA | ASP | A | 873 | 23.689 | 77.104 | 4.029 | 1.00 | 19.17 | A |
| ATOM | 6844 | CB | ASP | A | 873 | 24.112 | 77.329 | 5.482 | 1.00 | 19.04 | A |
| ATOM | 6845 | CG | ASP | A | 873 | 24.577 | 76.059 | 6.156 | 1.00 | 19.71 | A |
| ATOM | 6846 | OD1 | ASP | A | 873 | 23.811 | 75.502 | 6.971 | 1.00 | 19.93 | A |
| ATOM | 6847 | OD2 | ASP | A | 873 | 25.706 | 75.612 | 5.863 | 1.00 | 20.20 | A |
| ATOM | 6848 | C | ASP | A | 873 | 22.696 | 75.948 | 3.945 | 1.00 | 18.75 | A |
| ATOM | 6849 | O | ASP | A | 873 | 21.498 | 76.154 | 4.135 | 1.00 | 18.42 | A |
| ATOM | 6850 | N | ASP | A | 874 | 23.183 | 74.742 | 3.660 | 1.00 | 17.54 | A |
| ATOM | 6851 | CA | ASP | A | 874 | 22.299 | 73.585 | 3.551 | 1.00 | 17.33 | A |
| ATOM | 6852 | CB | ASP | A | 874 | 22.635 | 72.762 | 2.297 | 1.00 | 16.01 | A |
| ATOM | 6853 | CG | ASP | A | 874 | 24.122 | 72.502 | 2.138 | 1.00 | 16.32 | A |
| ATOM | 6854 | OD1 | ASP | A | 874 | 24.901 | 72.857 | 3.052 | 1.00 | 15.05 | A |
| ATOM | 6855 | OD2 | ASP | A | 874 | 24.506 | 71.936 | 1.091 | 1.00 | 16.01 | A |
| ATOM | 6856 | C | ASP | A | 874 | 22.248 | 72.681 | 4.788 | 1.00 | 17.97 | A |
| ATOM | 6857 | O | ASP | A | 874 | 22.029 | 71.476 | 4.685 | 1.00 | 18.00 | A |
| ATOM | 6858 | N | GLU | A | 875 | 22.460 | 73.284 | 5.953 | 1.00 | 18.50 | A |
| ATOM | 6859 | CA | GLU | A | 875 | 22.382 | 72.597 | 7.238 | 1.00 | 19.21 | A |
| ATOM | 6860 | CB | GLU | A | 875 | 20.910 | 72.346 | 7.585 | 1.00 | 21.54 | A |
| ATOM | 6861 | CG | GLU | A | 875 | 20.038 | 73.592 | 7.559 | 1.00 | 25.14 | A |
| ATOM | 6862 | CD | GLU | A | 875 | 18.589 | 73.289 | 7.885 | 1.00 | 27.52 | A |
| ATOM | 6863 | OE1 | GLU | A | 875 | 18.310 | 72.853 | 9.020 | 1.00 | 30.03 | A |
| ATOM | 6864 | OE2 | GLU | A | 875 | 17.727 | 73.479 | 7.003 | 1.00 | 30.33 | A |
| ATOM | 6865 | C | GLU | A | 875 | 23.150 | 71.295 | 7.451 | 1.00 | 18.48 | A |
| ATOM | 6866 | O | GLU | A | 875 | 22.627 | 70.367 | 8.070 | 1.00 | 17.86 | A |
| ATOM | 6867 | N | ARG | A | 876 | 24.380 | 71.208 | 6.956 | 1.00 | 17.24 | A |
| ATOM | 6868 | CA | ARG | A | 876 | 25.161 | 69.997 | 7.180 | 1.00 | 16.73 | A |
| ATOM | 6869 | CB | ARG | A | 876 | 25.517 | 69.320 | 5.848 | 1.00 | 16.77 | A |
| ATOM | 6870 | CG | ARG | A | 876 | 24.312 | 68.684 | 5.133 | 1.00 | 15.41 | A |
| ATOM | 6871 | CD | ARG | A | 876 | 23.606 | 67.656 | 6.027 | 1.00 | 15.04 | A |
| ATOM | 6872 | NE | ARG | A | 876 | 22.518 | 66.949 | 5.348 | 1.00 | 14.38 | A |
| ATOM | 6873 | CZ | ARG | A | 876 | 21.355 | 67.497 | 4.999 | 1.00 | 14.98 | A |
| ATOM | 6874 | NH1 | ARG | A | 876 | 21.107 | 68.776 | 5.259 | 1.00 | 12.60 | A |
| ATOM | 6875 | NH2 | ARG | A | 876 | 20.435 | 66.759 | 4.389 | 1.00 | 14.55 | A |
| ATOM | 6876 | C | ARG | A | 876 | 26.420 | 70.301 | 7.995 | 1.00 | 16.61 | A |
| ATOM | 6877 | O | ARG | A | 876 | 27.312 | 69.460 | 8.120 | 1.00 | 16.38 | A |
| ATOM | 6878 | N | GLY | A | 877 | 26.483 | 71.511 | 8.552 | 1.00 | 16.19 | A |
| ATOM | 6879 | CA | GLY | A | 877 | 27.619 | 71.887 | 9.375 | 1.00 | 16.15 | A |
| ATOM | 6880 | C | GLY | A | 877 | 28.605 | 72.908 | 8.829 | 1.00 | 15.84 | A |
| ATOM | 6881 | O | GLY | A | 877 | 29.426 | 73.435 | 9.586 | 1.00 | 16.37 | A |
| ATOM | 6882 | N | LEU | A | 878 | 28.537 | 73.198 | 7.534 | 1.00 | 16.26 | A |
| ATOM | 6883 | CA | LEU | A | 878 | 29.460 | 74.157 | 6.931 | 1.00 | 16.37 | A |
| ATOM | 6884 | CB | LEU | A | 878 | 29.297 | 74.159 | 5.410 | 1.00 | 15.68 | A |
| ATOM | 6885 | CG | LEU | A | 878 | 30.187 | 75.111 | 4.605 | 1.00 | 15.99 | A |
| ATOM | 6886 | CD1 | LEU | A | 878 | 31.649 | 74.925 | 4.994 | 1.00 | 15.30 | A |
| ATOM | 6887 | CD2 | LEU | A | 878 | 29.992 | 74.846 | 3.123 | 1.00 | 14.75 | A |
| ATOM | 6888 | C | LEU | A | 878 | 29.271 | 75.569 | 7.490 | 1.00 | 17.09 | A |
| ATOM | 6889 | O | LEU | A | 878 | 30.240 | 76.308 | 7.672 | 1.00 | 16.41 | A |
| ATOM | 6890 | N | GLY | A | 879 | 28.023 | 75.942 | 7.756 | 1.00 | 17.19 | A |
| ATOM | 6891 | CA | GLY | A | 879 | 27.757 | 77.257 | 8.315 | 1.00 | 18.15 | A |
| ATOM | 6892 | C | GLY | A | 879 | 27.833 | 78.419 | 7.342 | 1.00 | 18.91 | A |
| ATOM | 6893 | O | GLY | A | 879 | 27.929 | 79.574 | 7.760 | 1.00 | 19.29 | A |
| ATOM | 6894 | N | GLN | A | 880 | 27.798 | 78.122 | 6.047 | 1.00 | 18.66 | A |
| ATOM | 6895 | CA | GLN | A | 880 | 27.838 | 79.162 | 5.028 | 1.00 | 18.43 | A |
| ATOM | 6896 | CB | GLN | A | 880 | 29.244 | 79.774 | 4.910 | 1.00 | 18.45 | A |
| ATOM | 6897 | CG | GLN | A | 880 | 30.361 | 78.774 | 4.586 | 1.00 | 18.67 | A |
| ATOM | 6898 | CD | GLN | A | 880 | 31.589 | 79.432 | 3.963 | 1.00 | 18.32 | A |
| ATOM | 6899 | OE1 | GLN | A | 880 | 31.611 | 79.730 | 2.764 | 1.00 | 20.47 | A |
| ATOM | 6900 | NE2 | GLN | A | 880 | 32.609 | 79.670 | 4.776 | 1.00 | 16.53 | A |
| ATOM | 6901 | C | GLN | A | 880 | 27.424 | 78.615 | 3.670 | 1.00 | 18.38 | A |
| ATOM | 6902 | O | GLN | A | 880 | 27.389 | 77.399 | 3.455 | 1.00 | 18.09 | A |
| ATOM | 6903 | N | GLY | A | 881 | 27.095 | 79.527 | 2.763 | 1.00 | 18.10 | A |
| ATOM | 6904 | CA | GLY | A | 881 | 26.730 | 79.129 | 1.421 | 1.00 | 17.08 | A |
| ATOM | 6905 | C | GLY | A | 881 | 28.014 | 79.145 | 0.613 | 1.00 | 16.83 | A |
| ATOM | 6906 | O | GLY | A | 881 | 29.110 | 79.108 | 1.175 | 1.00 | 16.96 | A |
| ATOM | 6907 | N | VAL | A | 882 | 27.887 | 79.203 | −0.705 | 1.00 | 15.89 | A |
| ATOM | 6908 | CA | VAL | A | 882 | 29.045 | 79.236 | −1.578 | 1.00 | 15.83 | A |
| ATOM | 6909 | CB | VAL | A | 882 | 29.014 | 78.059 | −2.570 | 1.00 | 15.45 | A |
| ATOM | 6910 | CG1 | VAL | A | 882 | 30.229 | 78.105 | −3.476 | 1.00 | 15.71 | A |
| ATOM | 6911 | CG2 | VAL | A | 882 | 28.974 | 76.746 | −1.800 | 1.00 | 15.61 | A |
| ATOM | 6912 | C | VAL | A | 882 | 28.998 | 80.558 | −2.330 | 1.00 | 15.96 | A |
| ATOM | 6913 | O | VAL | A | 882 | 28.308 | 80.684 | −3.338 | 1.00 | 15.41 | A |
| ATOM | 6914 | N | LEU | A | 883 | 29.727 | 81.542 | −1.815 | 1.00 | 16.91 | A |
| ATOM | 6915 | CA | LEU | A | 883 | 29.761 | 82.872 | −2.415 | 1.00 | 17.79 | A |
| ATOM | 6916 | CB | LEU | A | 883 | 29.092 | 83.874 | −1.467 | 1.00 | 18.67 | A |
| ATOM | 6917 | CG | LEU | A | 883 | 27.586 | 83.717 | −1.225 | 1.00 | 19.39 | A |
| ATOM | 6918 | CD1 | LEU | A | 883 | 27.161 | 84.532 | −0.011 | 1.00 | 20.65 | A |
| ATOM | 6919 | CD2 | LEU | A | 883 | 26.829 | 84.178 | −2.453 | 1.00 | 20.59 | A |
| ATOM | 6920 | C | LEU | A | 883 | 31.184 | 83.331 | −2.726 | 1.00 | 17.96 | A |
| ATOM | 6921 | O | LEU | A | 883 | 31.420 | 84.518 | −2.953 | 1.00 | 19.51 | A |
| ATOM | 6922 | N | ASP | A | 884 | 32.126 | 82.393 | −2.745 | 1.00 | 17.04 | A |
| ATOM | 6923 | CA | ASP | A | 884 | 33.521 | 82.716 | −3.016 | 1.00 | 16.75 | A |
| ATOM | 6924 | CB | ASP | A | 884 | 34.422 | 82.110 | −1.934 | 1.00 | 17.01 | A |
| ATOM | 6925 | CG | ASP | A | 884 | 34.187 | 80.623 | −1.738 | 1.00 | 17.63 | A |
| ATOM | 6926 | OD1 | ASP | A | 884 | 34.839 | 80.044 | −0.842 | 1.00 | 17.95 | A |
| ATOM | 6927 | OD2 | ASP | A | 884 | 33.358 | 80.035 | −2.471 | 1.00 | 16.72 | A |
| ATOM | 6928 | C | ASP | A | 884 | 33.996 | 82.269 | −4.395 | 1.00 | 16.25 | A |
| ATOM | 6929 | O | ASP | A | 884 | 35.171 | 81.958 | −4.590 | 1.00 | 16.24 | A |
| ATOM | 6930 | N | ASN | A | 885 | 33.073 | 82.252 | −5.350 | 1.00 | 15.54 | A |
| ATOM | 6931 | CA | ASN | A | 885 | 33.380 | 81.857 | −6.719 | 1.00 | 15.55 | A |
| ATOM | 6932 | CB | ASN | A | 885 | 32.129 | 81.978 | −7.584 | 1.00 | 15.27 | A |
| ATOM | 6933 | CG | ASN | A | 885 | 30.959 | 81.217 | −7.014 | 1.00 | 16.04 | A |
| ATOM | 6934 | OD1 | ASN | A | 885 | 30.708 | 80.069 | −7.383 | 1.00 | 17.31 | A |
| ATOM | 6935 | ND2 | ASN | A | 885 | 30.244 | 81.846 | −6.089 | 1.00 | 14.69 | A |
| ATOM | 6936 | C | ASN | A | 885 | 34.464 | 82.737 | −7.316 | 1.00 | 15.81 | A |
| ATOM | 6937 | O | ASN | A | 885 | 34.593 | 83.910 | −6.964 | 1.00 | 15.21 | A |
| ATOM | 6938 | N | LYS | A | 886 | 35.234 | 82.163 | −8.231 | 1.00 | 15.55 | A |
| ATOM | 6939 | CA | LYS | A | 886 | 36.291 | 82.891 | −8.911 | 1.00 | 16.47 | A |
| ATOM | 6940 | CB | LYS | A | 886 | 37.616 | 82.746 | −8.154 | 1.00 | 18.07 | A |
| ATOM | 6941 | CG | LYS | A | 886 | 38.108 | 81.316 | −8.023 | 1.00 | 18.63 | A |
| ATOM | 6942 | CD | LYS | A | 886 | 39.241 | 81.199 | −7.009 | 1.00 | 20.71 | A |
| ATOM | 6943 | CE | LYS | A | 886 | 40.446 | 82.032 | −7.408 | 1.00 | 20.48 | A |
| ATOM | 6944 | NZ | LYS | A | 886 | 41.570 | 81.853 | −6.454 | 1.00 | 21.54 | A |
| ATOM | 6945 | C | LYS | A | 886 | 36.402 | 82.305 | −10.311 | 1.00 | 15.79 | A |
| ATOM | 6946 | O | LYS | A | 886 | 36.057 | 81.143 | −10.535 | 1.00 | 16.23 | A |
| ATOM | 6947 | N | PRO | A | 887 | 36.870 | 83.104 | −11.280 | 1.00 | 15.53 | A |
| ATOM | 6948 | CD | PRO | A | 887 | 37.247 | 84.527 | −11.189 | 1.00 | 15.01 | A |
| ATOM | 6949 | CA | PRO | A | 887 | 37.003 | 82.611 | −12.652 | 1.00 | 14.09 | A |
| ATOM | 6950 | CB | PRO | A | 887 | 37.711 | 83.762 | −13.363 | 1.00 | 15.12 | A |
| ATOM | 6951 | CG | PRO | A | 887 | 37.187 | 84.967 | −12.629 | 1.00 | 15.47 | A |
| ATOM | 6952 | C | PRO | A | 887 | 37.793 | 81.305 | −12.740 | 1.00 | 13.98 | A |
| ATOM | 6953 | O | PRO | A | 887 | 38.849 | 81.160 | −12.125 | 1.00 | 14.06 | A |
| ATOM | 6954 | N | VAL | A | 888 | 37.261 | 80.351 | −13.497 | 1.00 | 12.52 | A |
| ATOM | 6955 | CA | VAL | A | 888 | 37.927 | 79.073 | −13.687 | 1.00 | 12.09 | A |
| ATOM | 6956 | CB | VAL | A | 888 | 37.300 | 77.955 | −12.802 | 1.00 | 12.98 | A |
| ATOM | 6957 | CG1 | VAL | A | 888 | 35.794 | 77.879 | −13.026 | 1.00 | 13.02 | A |
| ATOM | 6958 | CG2 | VAL | A | 888 | 37.959 | 76.609 | −13.117 | 1.00 | 12.38 | A |
| ATOM | 6959 | C | VAL | A | 888 | 37.833 | 78.680 | −15.155 | 1.00 | 12.38 | A |
| ATOM | 6960 | O | VAL | A | 888 | 36.821 | 78.940 | −15.815 | 1.00 | 12.49 | A |
| ATOM | 6961 | N | LEU | A | 889 | 38.898 | 78.077 | −15.670 | 1.00 | 11.73 | A |
| ATOM | 6962 | CA | LEU | A | 889 | 38.918 | 77.638 | −17.056 | 1.00 | 11.82 | A |
| ATOM | 6963 | CB | LEU | A | 889 | 40.252 | 77.982 | −17.726 | 1.00 | 12.37 | A |
| ATOM | 6964 | CG | LEU | A | 889 | 40.314 | 77.579 | −19.208 | 1.00 | 12.93 | A |
| ATOM | 6965 | CD1 | LEU | A | 889 | 39.392 | 78.489 | −20.017 | 1.00 | 13.51 | A |
| ATOM | 6966 | CD2 | LEU | A | 889 | 41.745 | 77.675 | −19.728 | 1.00 | 14.04 | A |
| ATOM | 6967 | C | LEU | A | 889 | 38.712 | 76.129 | −17.114 | 1.00 | 11.67 | A |
| ATOM | 6968 | O | LEU | A | 889 | 39.629 | 75.365 | −16.818 | 1.00 | 11.61 | A |
| ATOM | 6969 | N | HIS | A | 890 | 37.503 | 75.708 | −17.475 | 1.00 | 11.59 | A |
| ATOM | 6970 | CA | HIS | A | 890 | 37.195 | 74.286 | −17.595 | 1.00 | 11.80 | A |
| ATOM | 6971 | CB | HIS | A | 890 | 35.699 | 74.024 | −17.405 | 1.00 | 11.62 | A |
| ATOM | 6972 | CG | HIS | A | 890 | 35.227 | 74.185 | −15.995 | 1.00 | 12.11 | A |
| ATOM | 6973 | CD2 | HIS | A | 890 | 34.057 | 74.648 | −15.495 | 1.00 | 11.32 | A |
| ATOM | 6974 | ND1 | HIS | A | 890 | 35.981 | 73.797 | −14.909 | 1.00 | 12.14 | A |
| ATOM | 6975 | CE1 | HIS | A | 890 | 35.296 | 74.014 | −13.800 | 1.00 | 12.02 | A |
| ATOM | 6976 | NE2 | HIS | A | 890 | 34.125 | 74.530 | −14.128 | 1.00 | 12.08 | A |
| ATOM | 6977 | C | HIS | A | 890 | 37.582 | 73.832 | −18.991 | 1.00 | 11.45 | A |
| ATOM | 6978 | O | HIS | A | 890 | 37.374 | 74.565 | −19.957 | 1.00 | 11.89 | A |
| ATOM | 6979 | N | ILE | A | 891 | 38.129 | 72.625 | −19.105 | 1.00 | 11.16 | A |
| ATOM | 6980 | CA | ILE | A | 891 | 38.527 | 72.109 | −20.409 | 1.00 | 10.51 | A |
| ATOM | 6981 | CB | ILE | A | 891 | 40.065 | 72.024 | −20.529 | 1.00 | 9.62 | A |
| ATOM | 6982 | CG2 | ILE | A | 891 | 40.674 | 73.405 | −20.275 | 1.00 | 10.29 | A |
| ATOM | 6983 | CG1 | ILE | A | 891 | 40.626 | 71.020 | −19.515 | 1.00 | 9.60 | A |
| ATOM | 6984 | CD1 | ILE | A | 891 | 42.118 | 70.735 | −19.709 | 1.00 | 9.63 | A |
| ATOM | 6985 | C | ILE | A | 891 | 37.910 | 70.740 | −20.685 | 1.00 | 10.45 | A |
| ATOM | 6986 | O | ILE | A | 891 | 37.677 | 69.956 | −19.764 | 1.00 | 11.02 | A |
| ATOM | 6987 | N | TYR | A | 892 | 37.644 | 70.467 | −21.959 | 1.00 | 9.88 | A |
| ATOM | 6988 | CA | TYR | A | 892 | 37.034 | 69.204 | −22.374 | 1.00 | 10.43 | A |
| ATOM | 6989 | CB | TYR | A | 892 | 35.499 | 69.319 | −22.415 | 1.00 | 10.07 | A |
| ATOM | 6990 | CG | TYR | A | 892 | 34.838 | 70.000 | −21.247 | 1.00 | 10.62 | A |
| ATOM | 6991 | CD1 | TYR | A | 892 | 34.810 | 71.395 | −21.142 | 1.00 | 9.84 | A |
| ATOM | 6992 | CE1 | TYR | A | 892 | 34.193 | 72.021 | −20.056 | 1.00 | 10.41 | A |
| ATOM | 6993 | CD2 | TYR | A | 892 | 34.234 | 69.249 | −20.241 | 1.00 | 10.00 | A |
| ATOM | 6994 | CE2 | TYR | A | 892 | 33.620 | 69.859 | −19.159 | 1.00 | 11.35 | A |
| ATOM | 6995 | CZ | TYR | A | 892 | 33.601 | 71.242 | −19.068 | 1.00 | 10.30 | A |
| ATOM | 6996 | OH | TYR | A | 892 | 32.989 | 71.830 | −17.986 | 1.00 | 10.40 | A |
| ATOM | 6997 | C | TYR | A | 892 | 37.446 | 68.806 | −23.781 | 1.00 | 10.21 | A |
| ATOM | 6998 | O | TYR | A | 892 | 38.067 | 69.576 | −24.505 | 1.00 | 9.47 | A |
| ATOM | 6999 | N | ARG | A | 893 | 37.082 | 67.580 | −24.151 | 1.00 | 11.39 | A |
| ATOM | 7000 | CA | ARG | A | 893 | 37.275 | 67.069 | −25.507 | 1.00 | 11.89 | A |
| ATOM | 7001 | CB | ARG | A | 893 | 38.353 | 65.978 | −25.595 | 1.00 | 11.65 | A |
| ATOM | 7002 | CG | ARG | A | 893 | 39.800 | 66.473 | −25.503 | 1.00 | 12.03 | A |
| ATOM | 7003 | CD | ARG | A | 893 | 40.146 | 67.534 | −26.559 | 1.00 | 12.81 | A |
| ATOM | 7004 | NE | ARG | A | 893 | 40.179 | 67.020 | −27.929 | 1.00 | 13.22 | A |
| ATOM | 7005 | CZ | ARG | A | 893 | 41.094 | 66.177 | −28.402 | 1.00 | 13.50 | A |
| ATOM | 7006 | NH1 | ARG | A | 893 | 42.072 | 65.734 | −27.620 | 1.00 | 13.17 | A |
| ATOM | 7007 | NH2 | ARG | A | 893 | 41.037 | 65.782 | −29.666 | 1.00 | 13.35 | A |
| ATOM | 7008 | C | ARG | A | 893 | 35.901 | 66.470 | −25.809 | 1.00 | 12.56 | A |
| ATOM | 7009 | O | ARG | A | 893 | 35.315 | 65.801 | −24.951 | 1.00 | 12.29 | A |
| ATOM | 7010 | N | LEU | A | 894 | 35.371 | 66.740 | −26.999 | 1.00 | 12.89 | A |
| ATOM | 7011 | CA | LEU | A | 894 | 34.061 | 66.224 | −27.387 | 1.00 | 13.29 | A |
| ATOM | 7012 | CB | LEU | A | 894 | 33.136 | 67.373 | −27.812 | 1.00 | 14.11 | A |
| ATOM | 7013 | CG | LEU | A | 894 | 31.689 | 66.956 | −28.119 | 1.00 | 13.84 | A |
| ATOM | 7014 | CD1 | LEU | A | 894 | 31.050 | 66.397 | −26.859 | 1.00 | 15.01 | A |
| ATOM | 7015 | CD2 | LEU | A | 894 | 30.885 | 68.139 | −28.631 | 1.00 | 14.69 | A |
| ATOM | 7016 | C | LEU | A | 894 | 34.254 | 65.245 | −28.541 | 1.00 | 13.05 | A |
| ATOM | 7017 | O | LEU | A | 894 | 34.643 | 65.635 | −29.642 | 1.00 | 13.01 | A |
| ATOM | 7018 | N | VAL | A | 895 | 33.976 | 63.972 | −28.278 | 1.00 | 13.11 | A |
| ATOM | 7019 | CA | VAL | A | 895 | 34.166 | 62.927 | −29.273 | 1.00 | 14.21 | A |
| ATOM | 7020 | CB | VAL | A | 895 | 35.048 | 61.789 | −28.711 | 1.00 | 14.02 | A |
| ATOM | 7021 | CG1 | VAL | A | 895 | 35.395 | 60.802 | −29.822 | 1.00 | 13.83 | A |
| ATOM | 7022 | CG2 | VAL | A | 895 | 36.304 | 62.361 | −28.072 | 1.00 | 14.27 | A |
| ATOM | 7023 | C | VAL | A | 895 | 32.884 | 62.281 | −29.788 | 1.00 | 14.24 | A |
| ATOM | 7024 | O | VAL | A | 895 | 32.204 | 61.573 | −29.047 | 1.00 | 14.21 | A |
| ATOM | 7025 | N | LEU | A | 896 | 32.565 | 62.525 | −31.056 | 1.00 | 14.43 | A |
| ATOM | 7026 | CA | LEU | A | 896 | 31.397 | 61.912 | −31.690 | 1.00 | 15.16 | A |
| ATOM | 7027 | CB | LEU | A | 896 | 30.692 | 62.896 | −32.632 | 1.00 | 16.09 | A |
| ATOM | 7028 | CG | LEU | A | 896 | 29.533 | 62.295 | −33.441 | 1.00 | 16.60 | A |
| ATOM | 7029 | CD1 | LEU | A | 896 | 28.388 | 61.921 | −32.507 | 1.00 | 16.94 | A |
| ATOM | 7030 | CD2 | LEU | A | 896 | 29.055 | 63.298 | −34.489 | 1.00 | 17.67 | A |
| ATOM | 7031 | C | LEU | A | 896 | 31.967 | 60.746 | −32.498 | 1.00 | 15.44 | A |
| ATOM | 7032 | O | LEU | A | 896 | 32.934 | 60.924 | −33.240 | 1.00 | 15.11 | A |
| ATOM | 7033 | N | GLU | A | 897 | 31.382 | 59.560 | −32.348 | 1.00 | 15.78 | A |
| ATOM | 7034 | CA | GLU | A | 897 | 31.882 | 58.379 | −33.052 | 1.00 | 16.64 | A |
| ATOM | 7035 | CB | GLU | A | 897 | 32.838 | 57.591 | −32.157 | 1.00 | 17.33 | A |
| ATOM | 7036 | CG | GLU | A | 897 | 34.005 | 58.347 | −31.591 | 1.00 | 18.67 | A |
| ATOM | 7037 | CD | GLU | A | 897 | 34.770 | 57.506 | −30.583 | 1.00 | 18.78 | A |
| ATOM | 7038 | OE1 | GLU | A | 897 | 34.189 | 57.165 | −29.530 | 1.00 | 20.08 | A |
| ATOM | 7039 | OE2 | GLU | A | 897 | 35.941 | 57.180 | −30.850 | 1.00 | 20.18 | A |
| ATOM | 7040 | C | GLU | A | 897 | 30.789 | 57.406 | −33.455 | 1.00 | 16.86 | A |
| ATOM | 7041 | O | GLU | A | 897 | 29.731 | 57.356 | −32.834 | 1.00 | 16.37 | A |
| ATOM | 7042 | N | LYS | A | 898 | 31.070 | 56.620 | −34.491 | 1.00 | 17.54 | A |
| ATOM | 7043 | CA | LYS | A | 898 | 30.149 | 55.583 | −34.935 | 1.00 | 18.69 | A |
| ATOM | 7044 | CB | LYS | A | 898 | 30.296 | 55.327 | −36.438 | 1.00 | 19.99 | A |
| ATOM | 7045 | CG | LYS | A | 898 | 29.903 | 56.506 | −37.323 | 1.00 | 21.82 | A |
| ATOM | 7046 | CD | LYS | A | 898 | 28.480 | 56.982 | −37.034 | 1.00 | 24.25 | A |
| ATOM | 7047 | CE | LYS | A | 898 | 27.461 | 55.858 | −37.177 | 1.00 | 25.50 | A |
| ATOM | 7048 | NZ | LYS | A | 898 | 26.080 | 56.336 | −36.892 | 1.00 | 27.51 | A |
| ATOM | 7049 | C | LYS | A | 898 | 30.619 | 54.359 | −34.145 | 1.00 | 18.71 | A |
| ATOM | 7050 | O | LYS | A | 898 | 31.815 | 54.072 | −34.095 | 1.00 | 19.42 | A |
| ATOM | 7051 | N | VAL | A | 899 | 29.691 | 53.647 | −33.517 | 1.00 | 18.14 | A |
| ATOM | 7052 | CA | VAL | A | 899 | 30.062 | 52.486 | −32.720 | 1.00 | 17.77 | A |
| ATOM | 7053 | CB | VAL | A | 899 | 29.804 | 52.759 | −31.219 | 1.00 | 17.74 | A |
| ATOM | 7054 | CG1 | VAL | A | 899 | 30.749 | 53.847 | −30.719 | 1.00 | 17.93 | A |
| ATOM | 7055 | CG2 | VAL | A | 899 | 28.354 | 53.189 | −31.009 | 1.00 | 17.25 | A |
| ATOM | 7056 | C | VAL | A | 899 | 29.329 | 51.204 | −33.117 | 1.00 | 17.99 | A |
| ATOM | 7057 | O | VAL | A | 899 | 29.343 | 50.225 | −32.375 | 1.00 | 16.80 | A |
| ATOM | 7058 | N | ASN | A | 900 | 28.696 | 51.204 | −34.286 | 1.00 | 18.17 | A |
| ATOM | 7059 | CA | ASN | A | 900 | 27.960 | 50.022 | −34.727 | 1.00 | 19.54 | A |
| ATOM | 7060 | CB | ASN | A | 900 | 27.149 | 50.336 | −35.993 | 1.00 | 21.01 | A |
| ATOM | 7061 | CG | ASN | A | 900 | 27.997 | 50.923 | −37.096 | 1.00 | 22.59 | A |
| ATOM | 7062 | OD1 | ASN | A | 900 | 28.594 | 51.989 | −36.935 | 1.00 | 24.04 | A |
| ATOM | 7063 | ND2 | ASN | A | 900 | 28.058 | 50.231 | −38.230 | 1.00 | 23.54 | A |
| ATOM | 7064 | C | ASN | A | 900 | 28.858 | 48.813 | −34.979 | 1.00 | 19.41 | A |
| ATOM | 7065 | O | ASN | A | 900 | 28.390 | 47.676 | −34.950 | 1.00 | 19.92 | A |
| ATOM | 7066 | N | ASN | A | 901 | 30.142 | 49.048 | −35.226 | 1.00 | 18.98 | A |
| ATOM | 7067 | CA | ASN | A | 901 | 31.067 | 47.946 | −35.478 | 1.00 | 19.29 | A |
| ATOM | 7068 | CB | ASN | A | 901 | 32.053 | 48.310 | −36.589 | 1.00 | 20.87 | A |
| ATOM | 7069 | CG | ASN | A | 901 | 31.401 | 48.359 | −37.948 | 1.00 | 23.08 | A |
| ATOM | 7070 | OD1 | ASN | A | 901 | 30.649 | 47.459 | −38.315 | 1.00 | 24.77 | A |
| ATOM | 7071 | ND2 | ASN | A | 901 | 31.695 | 49.407 | −38.712 | 1.00 | 24.33 | A |
| ATOM | 7072 | C | ASN | A | 901 | 31.855 | 47.535 | −34.243 | 1.00 | 18.73 | A |
| ATOM | 7073 | O | ASN | A | 901 | 32.631 | 46.583 | −34.288 | 1.00 | 18.51 | A |
| ATOM | 7074 | N | CYS | A | 902 | 31.660 | 48.251 | −33.142 | 1.00 | 17.77 | A |
| ATOM | 7075 | CA | CYS | A | 902 | 32.382 | 47.944 | −31.913 | 1.00 | 17.80 | A |
| ATOM | 7076 | C | CYS | A | 902 | 31.806 | 46.754 | −31.162 | 1.00 | 17.57 | A |
| ATOM | 7077 | O | CYS | A | 902 | 30.591 | 46.581 | −31.087 | 1.00 | 18.35 | A |
| ATOM | 7078 | CB | CYS | A | 902 | 32.370 | 49.140 | −30.963 | 1.00 | 18.11 | A |
| ATOM | 7079 | SG | CYS | A | 902 | 33.114 | 50.689 | −31.563 | 1.00 | 18.40 | A |
| ATOM | 7080 | N | VAL | A | 903 | 32.688 | 45.941 | −30.596 | 1.00 | 17.18 | A |
| ATOM | 7081 | CA | VAL | A | 903 | 32.258 | 44.795 | −29.809 | 1.00 | 16.95 | A |
| ATOM | 7082 | CB | VAL | A | 903 | 33.354 | 43.719 | −29.739 | 1.00 | 16.42 | A |
| ATOM | 7083 | CG1 | VAL | A | 903 | 32.911 | 42.586 | −28.820 | 1.00 | 16.29 | A |
| ATOM | 7084 | CG2 | VAL | A | 903 | 33.643 | 43.188 | −31.134 | 1.00 | 16.61 | A |
| ATOM | 7085 | C | VAL | A | 903 | 31.993 | 45.345 | −28.411 | 1.00 | 17.40 | A |
| ATOM | 7086 | O | VAL | A | 903 | 32.924 | 45.634 | −27.659 | 1.00 | 17.19 | A |
| ATOM | 7087 | N | ARG | A | 904 | 30.721 | 45.508 | −28.076 | 1.00 | 17.57 | A |
| ATOM | 7088 | CA | ARG | A | 904 | 30.339 | 46.054 | −26.781 | 1.00 | 19.09 | A |
| ATOM | 7089 | CB | ARG | A | 904 | 29.312 | 47.174 | −26.985 | 1.00 | 19.60 | A |
| ATOM | 7090 | CG | ARG | A | 904 | 29.902 | 48.417 | −27.636 | 1.00 | 20.55 | A |
| ATOM | 7091 | CD | ARG | A | 904 | 28.834 | 49.450 | −27.962 | 1.00 | 22.04 | A |
| ATOM | 7092 | NE | ARG | A | 904 | 28.034 | 49.070 | −29.123 | 1.00 | 22.98 | A |
| ATOM | 7093 | CZ | ARG | A | 904 | 27.079 | 49.835 | −29.649 | 1.00 | 24.10 | A |
| ATOM | 7094 | NH1 | ARG | A | 904 | 26.807 | 51.018 | −29.114 | 1.00 | 23.36 | A |
| ATOM | 7095 | NH2 | ARG | A | 904 | 26.402 | 49.421 | −30.713 | 1.00 | 23.49 | A |
| ATOM | 7096 | C | ARG | A | 904 | 29.778 | 45.010 | −25.826 | 1.00 | 18.91 | A |
| ATOM | 7097 | O | ARG | A | 904 | 29.445 | 43.895 | −26.233 | 1.00 | 19.16 | A |
| ATOM | 7098 | N | PRO | A | 905 | 29.684 | 45.355 | −24.531 | 1.00 | 18.86 | A |
| ATOM | 7099 | CD | PRO | A | 905 | 30.225 | 46.564 | −23.885 | 1.00 | 18.98 | A |
| ATOM | 7100 | CA | PRO | A | 905 | 29.152 | 44.427 | −23.530 | 1.00 | 18.75 | A |
| ATOM | 7101 | CB | PRO | A | 905 | 29.356 | 45.177 | −22.217 | 1.00 | 18.15 | A |
| ATOM | 7102 | CG | PRO | A | 905 | 30.531 | 46.064 | −22.503 | 1.00 | 18.29 | A |
| ATOM | 7103 | C | PRO | A | 905 | 27.677 | 44.182 | −23.811 | 1.00 | 19.11 | A |
| ATOM | 7104 | O | PRO | A | 905 | 27.020 | 45.011 | −24.447 | 1.00 | 18.48 | A |
| ATOM | 7105 | N | SER | A | 906 | 27.156 | 43.052 | −23.339 | 1.00 | 19.65 | A |
| ATOM | 7106 | CA | SER | A | 906 | 25.747 | 42.741 | −23.548 | 1.00 | 20.62 | A |
| ATOM | 7107 | CB | SER | A | 906 | 25.405 | 41.351 | −23.011 | 1.00 | 21.91 | A |
| ATOM | 7108 | OG | SER | A | 906 | 25.224 | 41.388 | −21.605 | 1.00 | 23.95 | A |
| ATOM | 7109 | C | SER | A | 906 | 24.911 | 43.774 | −22.809 | 1.00 | 20.80 | A |
| ATOM | 7110 | O | SER | A | 906 | 25.427 | 44.536 | −21.988 | 1.00 | 19.32 | A |
| ATOM | 7111 | N | LYS | A | 907 | 23.615 | 43.782 | −23.095 | 1.00 | 21.12 | A |
| ATOM | 7112 | CA | LYS | A | 907 | 22.692 | 44.721 | −22.474 | 1.00 | 22.75 | A |
| ATOM | 7113 | CB | LYS | A | 907 | 21.291 | 44.523 | −23.063 | 1.00 | 25.21 | A |
| ATOM | 7114 | CG | LYS | A | 907 | 21.187 | 44.877 | −24.541 | 1.00 | 28.17 | A |
| ATOM | 7115 | CD | LYS | A | 907 | 19.869 | 44.394 | −25.156 | 1.00 | 30.78 | A |
| ATOM | 7116 | CE | LYS | A | 907 | 18.652 | 44.921 | −24.398 | 1.00 | 32.30 | A |
| ATOM | 7117 | NZ | LYS | A | 907 | 18.558 | 46.411 | −24.413 | 1.00 | 34.30 | A |
| ATOM | 7118 | C | LYS | A | 907 | 22.631 | 44.606 | −20.952 | 1.00 | 21.68 | A |
| ATOM | 7119 | O | LYS | A | 907 | 22.243 | 45.552 | −20.272 | 1.00 | 22.39 | A |
| ATOM | 7120 | N | LEU | A | 908 | 23.023 | 43.454 | −20.417 | 1.00 | 20.87 | A |
| ATOM | 7121 | CA | LEU | A | 908 | 22.972 | 43.237 | −18.974 | 1.00 | 20.13 | A |
| ATOM | 7122 | CB | LEU | A | 908 | 22.543 | 41.793 | −18.686 | 1.00 | 21.33 | A |
| ATOM | 7123 | CG | LEU | A | 908 | 21.169 | 41.386 | −19.238 | 1.00 | 22.83 | A |
| ATOM | 7124 | CD1 | LEU | A | 908 | 20.891 | 39.928 | −18.905 | 1.00 | 22.96 | A |
| ATOM | 7125 | CD2 | LEU | A | 908 | 20.081 | 42.285 | −18.650 | 1.00 | 23.40 | A |
| ATOM | 7126 | C | LEU | A | 908 | 24.271 | 43.549 | −18.226 | 1.00 | 18.65 | A |
| ATOM | 7127 | O | LEU | A | 908 | 24.307 | 43.506 | −16.996 | 1.00 | 18.58 | A |
| ATOM | 7128 | N | HIS | A | 909 | 25.330 | 43.864 | −18.962 | 1.00 | 17.27 | A |
| ATOM | 7129 | CA | HIS | A | 909 | 26.618 | 44.178 | −18.345 | 1.00 | 16.78 | A |
| ATOM | 7130 | CB | HIS | A | 909 | 27.719 | 44.152 | −19.409 | 1.00 | 16.00 | A |
| ATOM | 7131 | CG | HIS | A | 909 | 29.093 | 43.939 | −18.857 | 1.00 | 16.16 | A |
| ATOM | 7132 | CD2 | HIS | A | 909 | 29.923 | 42.871 | −18.927 | 1.00 | 15.42 | A |
| ATOM | 7133 | ND1 | HIS | A | 909 | 29.764 | 44.899 | −18.129 | 1.00 | 15.64 | A |
| ATOM | 7134 | CE1 | HIS | A | 909 | 30.948 | 44.432 | −17.776 | 1.00 | 16.06 | A |
| ATOM | 7135 | NE2 | HIS | A | 909 | 31.070 | 43.203 | −18.249 | 1.00 | 16.03 | A |
| ATOM | 7136 | C | HIS | A | 909 | 26.535 | 45.561 | −17.688 | 1.00 | 15.94 | A |
| ATOM | 7137 | O | HIS | A | 909 | 26.027 | 46.509 | −18.286 | 1.00 | 15.96 | A |
| ATOM | 7138 | N | PRO | A | 910 | 27.028 | 45.689 | −16.445 | 1.00 | 15.17 | A |
| ATOM | 7139 | CD | PRO | A | 910 | 27.462 | 44.595 | −15.561 | 1.00 | 15.87 | A |
| ATOM | 7140 | CA | PRO | A | 910 | 27.004 | 46.958 | −15.706 | 1.00 | 14.56 | A |
| ATOM | 7141 | CB | PRO | A | 910 | 27.193 | 46.525 | −14.246 | 1.00 | 14.78 | A |
| ATOM | 7142 | CG | PRO | A | 910 | 26.902 | 45.041 | −14.247 | 1.00 | 15.77 | A |
| ATOM | 7143 | C | PRO | A | 910 | 28.076 | 47.971 | −16.108 | 1.00 | 13.94 | A |
| ATOM | 7144 | O | PRO | A | 910 | 28.051 | 49.112 | −15.642 | 1.00 | 14.38 | A |
| ATOM | 7145 | N | ALA | A | 911 | 29.016 | 47.561 | −16.952 | 1.00 | 13.17 | A |
| ATOM | 7146 | CA | ALA | A | 911 | 30.095 | 48.455 | −17.354 | 1.00 | 13.46 | A |
| ATOM | 7147 | CB | ALA | A | 911 | 31.445 | 47.784 | −17.086 | 1.00 | 13.95 | A |
| ATOM | 7148 | C | ALA | A | 911 | 30.049 | 48.933 | −18.797 | 1.00 | 12.94 | A |
| ATOM | 7149 | O | ALA | A | 911 | 29.284 | 48.431 | −19.621 | 1.00 | 12.72 | A |
| ATOM | 7150 | N | GLY | A | 912 | 30.898 | 49.914 | −19.081 | 1.00 | 13.06 | A |
| ATOM | 7151 | CA | GLY | A | 912 | 31.019 | 50.468 | −20.415 | 1.00 | 13.02 | A |
| ATOM | 7152 | C | GLY | A | 912 | 32.493 | 50.752 | −20.614 | 1.00 | 13.32 | A |
| ATOM | 7153 | O | GLY | A | 912 | 33.223 | 50.894 | −19.630 | 1.00 | 13.24 | A |
| ATOM | 7154 | N | TYR | A | 913 | 32.941 | 50.830 | −21.865 | 1.00 | 12.99 | A |
| ATOM | 7155 | CA | TYR | A | 913 | 34.348 | 51.097 | −22.148 | 1.00 | 13.02 | A |
| ATOM | 7156 | CB | TYR | A | 913 | 35.067 | 49.804 | −22.546 | 1.00 | 13.52 | A |
| ATOM | 7157 | CG | TYR | A | 913 | 35.020 | 48.754 | −21.462 | 1.00 | 13.68 | A |
| ATOM | 7158 | CD1 | TYR | A | 913 | 34.023 | 47.782 | −21.451 | 1.00 | 14.79 | A |
| ATOM | 7159 | CE1 | TYR | A | 913 | 33.927 | 46.864 | −20.409 | 1.00 | 14.26 | A |
| ATOM | 7160 | CD2 | TYR | A | 913 | 35.929 | 48.780 | −20.406 | 1.00 | 13.81 | A |
| ATOM | 7161 | CE2 | TYR | A | 913 | 35.842 | 47.868 | −19.358 | 1.00 | 14.11 | A |
| ATOM | 7162 | CZ | TYR | A | 913 | 34.835 | 46.916 | −19.367 | 1.00 | 14.91 | A |
| ATOM | 7163 | OH | TYR | A | 913 | 34.717 | 46.030 | −18.322 | 1.00 | 15.05 | A |
| ATOM | 7164 | C | TYR | A | 913 | 34.536 | 52.135 | −23.243 | 1.00 | 12.69 | A |
| ATOM | 7165 | O | TYR | A | 913 | 33.729 | 52.235 | −24.164 | 1.00 | 12.95 | A |
| ATOM | 7166 | N | LEU | A | 914 | 35.614 | 52.904 | −23.130 | 1.00 | 12.39 | A |
| ATOM | 7167 | CA | LEU | A | 914 | 35.934 | 53.934 | −24.110 | 1.00 | 11.88 | A |
| ATOM | 7168 | CB | LEU | A | 914 | 36.920 | 54.953 | −23.523 | 1.00 | 10.80 | A |
| ATOM | 7169 | CG | LEU | A | 914 | 36.488 | 55.845 | −22.354 | 1.00 | 11.21 | A |
| ATOM | 7170 | CD1 | LEU | A | 914 | 37.606 | 56.837 | −22.050 | 1.00 | 10.84 | A |
| ATOM | 7171 | CD2 | LEU | A | 914 | 35.199 | 56.589 | −22.701 | 1.00 | 11.02 | A |
| ATOM | 7172 | C | LEU | A | 914 | 36.563 | 53.342 | −25.361 | 1.00 | 12.53 | A |
| ATOM | 7173 | O | LEU | A | 914 | 37.001 | 52.185 | −25.380 | 1.00 | 11.85 | A |
| ATOM | 7174 | N | THR | A | 915 | 36.594 | 54.162 | −26.406 | 1.00 | 12.37 | A |
| ATOM | 7175 | CA | THR | A | 915 | 37.207 | 53.810 | −27.674 | 1.00 | 12.90 | A |
| ATOM | 7176 | CB | THR | A | 915 | 36.520 | 54.514 | −28.850 | 1.00 | 13.18 | A |
| ATOM | 7177 | OG1 | THR | A | 915 | 36.487 | 55.923 | −28.588 | 1.00 | 14.07 | A |
| ATOM | 7178 | CG2 | THR | A | 915 | 35.107 | 53.998 | −29.050 | 1.00 | 13.81 | A |
| ATOM | 7179 | C | THR | A | 915 | 38.609 | 54.390 | −27.571 | 1.00 | 13.07 | A |
| ATOM | 7180 | O | THR | A | 915 | 38.890 | 55.184 | −26.674 | 1.00 | 12.57 | A |
| ATOM | 7181 | N | SER | A | 916 | 39.479 | 54.004 | −28.494 | 1.00 | 13.02 | A |
| ATOM | 7182 | CA | SER | A | 916 | 40.846 | 54.504 | −28.517 | 1.00 | 13.32 | A |
| ATOM | 7183 | CB | SER | A | 916 | 41.584 | 53.933 | −29.727 | 1.00 | 14.65 | A |
| ATOM | 7184 | OG | SER | A | 916 | 42.822 | 54.591 | −29.917 | 1.00 | 18.31 | A |
| ATOM | 7185 | C | SER | A | 916 | 40.883 | 56.031 | −28.580 | 1.00 | 13.17 | A |
| ATOM | 7186 | O | SER | A | 916 | 41.628 | 56.672 | −27.842 | 1.00 | 11.89 | A |
| ATOM | 7187 | N | ALA | A | 917 | 40.074 | 56.611 | −29.463 | 1.00 | 12.45 | A |
| ATOM | 7188 | CA | ALA | A | 917 | 40.043 | 58.065 | −29.620 | 1.00 | 12.29 | A |
| ATOM | 7189 | CB | ALA | A | 917 | 39.113 | 58.451 | −30.764 | 1.00 | 13.11 | A |
| ATOM | 7190 | C | ALA | A | 917 | 39.609 | 58.771 | −28.343 | 1.00 | 12.12 | A |
| ATOM | 7191 | O | ALA | A | 917 | 40.201 | 59.779 | −27.951 | 1.00 | 11.03 | A |
| ATOM | 7192 | N | ALA | A | 918 | 38.569 | 58.248 | −27.702 | 1.00 | 11.73 | A |
| ATOM | 7193 | CA | ALA | A | 918 | 38.060 | 58.851 | −26.474 | 1.00 | 11.66 | A |
| ATOM | 7194 | CB | ALA | A | 918 | 36.761 | 58.174 | −26.062 | 1.00 | 11.10 | A |
| ATOM | 7195 | C | ALA | A | 918 | 39.089 | 58.747 | −25.353 | 1.00 | 11.78 | A |
| ATOM | 7196 | O | ALA | A | 918 | 39.261 | 59.675 | −24.555 | 1.00 | 11.86 | A |
| ATOM | 7197 | N | HIS | A | 919 | 39.773 | 57.612 | −25.289 | 1.00 | 10.86 | A |
| ATOM | 7198 | CA | HIS | A | 919 | 40.785 | 57.409 | −24.262 | 1.00 | 11.84 | A |
| ATOM | 7199 | CB | HIS | A | 919 | 41.281 | 55.959 | −24.287 | 1.00 | 11.67 | A |
| ATOM | 7200 | CG | HIS | A | 919 | 42.386 | 55.683 | −23.315 | 1.00 | 13.50 | A |
| ATOM | 7201 | CD2 | HIS | A | 919 | 42.420 | 55.735 | −21.963 | 1.00 | 13.61 | A |
| ATOM | 7202 | ND1 | HIS | A | 919 | 43.658 | 55.331 | −23.716 | 1.00 | 13.89 | A |
| ATOM | 7203 | CE1 | HIS | A | 919 | 44.427 | 55.180 | −22.654 | 1.00 | 13.39 | A |
| ATOM | 7204 | NE2 | HIS | A | 919 | 43.701 | 55.420 | −21.577 | 1.00 | 14.91 | A |
| ATOM | 7205 | C | HIS | A | 919 | 41.952 | 58.376 | −24.473 | 1.00 | 11.88 | A |
| ATOM | 7206 | O | HIS | A | 919 | 42.399 | 59.037 | −23.532 | 1.00 | 11.54 | A |
| ATOM | 7207 | N | LYS | A | 920 | 42.440 | 58.473 | −25.706 | 1.00 | 11.97 | A |
| ATOM | 7208 | CA | LYS | A | 920 | 43.547 | 59.378 | −25.979 | 1.00 | 12.11 | A |
| ATOM | 7209 | CB | LYS | A | 920 | 44.042 | 59.215 | −27.421 | 1.00 | 13.63 | A |
| ATOM | 7210 | CG | LYS | A | 920 | 44.909 | 57.967 | −27.617 | 1.00 | 14.69 | A |
| ATOM | 7211 | CD | LYS | A | 920 | 45.591 | 57.946 | −28.978 | 1.00 | 15.57 | A |
| ATOM | 7212 | CE | LYS | A | 920 | 46.543 | 56.769 | −29.089 | 1.00 | 15.56 | A |
| ATOM | 7213 | NZ | LYS | A | 920 | 47.665 | 56.868 | −28.111 | 1.00 | 16.03 | A |
| ATOM | 7214 | C | LYS | A | 920 | 43.145 | 60.823 | −25.712 | 1.00 | 11.83 | A |
| ATOM | 7215 | O | LYS | A | 920 | 43.962 | 61.623 | −25.251 | 1.00 | 11.38 | A |
| ATOM | 7216 | N | ALA | A | 921 | 41.886 | 61.154 | −25.989 | 1.00 | 11.22 | A |
| ATOM | 7217 | CA | ALA | A | 921 | 41.399 | 62.513 | −25.757 | 1.00 | 11.42 | A |
| ATOM | 7218 | CB | ALA | A | 921 | 39.972 | 62.664 | −26.297 | 1.00 | 10.82 | A |
| ATOM | 7219 | C | ALA | A | 921 | 41.443 | 62.810 | −24.256 | 1.00 | 11.71 | A |
| ATOM | 7220 | O | ALA | A | 921 | 41.803 | 63.914 | −23.841 | 1.00 | 12.03 | A |
| ATOM | 7221 | N | SER | A | 922 | 41.085 | 61.824 | −23.439 | 1.00 | 10.96 | A |
| ATOM | 7222 | CA | SER | A | 922 | 41.120 | 62.015 | −21.992 | 1.00 | 10.63 | A |
| ATOM | 7223 | CB | SER | A | 922 | 40.574 | 60.783 | −21.263 | 1.00 | 10.56 | A |
| ATOM | 7224 | OG | SER | A | 922 | 40.661 | 60.964 | −19.855 | 1.00 | 9.29 | A |
| ATOM | 7225 | C | SER | A | 922 | 42.560 | 62.257 | −21.547 | 1.00 | 10.95 | A |
| ATOM | 7226 | O | SER | A | 922 | 42.824 | 63.116 | −20.709 | 1.00 | 11.72 | A |
| ATOM | 7227 | N | GLN | A | 923 | 43.493 | 61.498 | −22.113 | 1.00 | 11.31 | A |
| ATOM | 7228 | CA | GLN | A | 923 | 44.897 | 61.650 | −21.755 | 1.00 | 11.08 | A |
| ATOM | 7229 | CB | GLN | A | 923 | 45.740 | 60.551 | −22.404 | 1.00 | 11.10 | A |
| ATOM | 7230 | CG | GLN | A | 923 | 45.413 | 59.139 | −21.923 | 1.00 | 10.78 | A |
| ATOM | 7231 | CD | GLN | A | 923 | 46.344 | 58.104 | −22.518 | 1.00 | 11.62 | A |
| ATOM | 7232 | OE1 | GLN | A | 923 | 46.500 | 58.031 | −23.738 | 1.00 | 12.23 | A |
| ATOM | 7233 | NE2 | GLN | A | 923 | 46.973 | 57.294 | −21.659 | 1.00 | 11.02 | A |
| ATOM | 7234 | C | GLN | A | 923 | 45.433 | 63.023 | −22.157 | 1.00 | 11.58 | A |
| ATOM | 7235 | O | GLN | A | 923 | 46.312 | 63.566 | −21.486 | 1.00 | 11.23 | A |
| ATOM | 7236 | N | SER | A | 924 | 44.902 | 63.585 | −23.241 | 1.00 | 11.01 | A |
| ATOM | 7237 | CA | SER | A | 924 | 45.343 | 64.900 | −23.704 | 1.00 | 12.31 | A |
| ATOM | 7238 | CB | SER | A | 924 | 44.727 | 65.236 | −25.069 | 1.00 | 13.21 | A |
| ATOM | 7239 | OG | SER | A | 924 | 43.363 | 65.616 | −24.936 | 1.00 | 13.77 | A |
| ATOM | 7240 | C | SER | A | 924 | 44.948 | 65.981 | −22.702 | 1.00 | 12.26 | A |
| ATOM | 7241 | O | SER | A | 924 | 45.590 | 67.030 | −22.625 | 1.00 | 13.44 | A |
| ATOM | 7242 | N | LEU | A | 925 | 43.886 | 65.728 | −21.944 | 1.00 | 11.76 | A |
| ATOM | 7243 | CA | LEU | A | 925 | 43.408 | 66.684 | −20.950 | 1.00 | 11.62 | A |
| ATOM | 7244 | CB | LEU | A | 925 | 41.892 | 66.533 | −20.759 | 1.00 | 12.13 | A |
| ATOM | 7245 | CG | LEU | A | 925 | 41.000 | 66.792 | −21.977 | 1.00 | 11.95 | A |
| ATOM | 7246 | CD1 | LEU | A | 925 | 39.552 | 66.421 | −21.645 | 1.00 | 11.65 | A |
| ATOM | 7247 | CD2 | LEU | A | 925 | 41.101 | 68.252 | −22.387 | 1.00 | 12.24 | A |
| ATOM | 7248 | C | LEU | A | 925 | 44.092 | 66.524 | −19.593 | 1.00 | 11.32 | A |
| ATOM | 7249 | O | LEU | A | 925 | 44.483 | 67.507 | −18.962 | 1.00 | 11.10 | A |
| ATOM | 7250 | N | LEU | A | 926 | 44.240 | 65.282 | −19.148 | 1.00 | 10.58 | A |
| ATOM | 7251 | CA | LEU | A | 926 | 44.837 | 65.021 | −17.843 | 1.00 | 10.87 | A |
| ATOM | 7252 | CB | LEU | A | 926 | 44.268 | 63.725 | −17.257 | 1.00 | 10.32 | A |
| ATOM | 7253 | CG | LEU | A | 926 | 42.756 | 63.720 | −17.008 | 1.00 | 11.66 | A |
| ATOM | 7254 | CD1 | LEU | A | 926 | 42.337 | 62.385 | −16.410 | 1.00 | 11.49 | A |
| ATOM | 7255 | CD2 | LEU | A | 926 | 42.385 | 64.867 | −16.070 | 1.00 | 12.14 | A |
| ATOM | 7256 | C | LEU | A | 926 | 46.356 | 64.961 | −17.808 | 1.00 | 11.34 | A |
| ATOM | 7257 | O | LEU | A | 926 | 46.962 | 65.305 | −16.795 | 1.00 | 11.49 | A |
| ATOM | 7258 | N | ASP | A | 927 | 46.975 | 64.519 | −18.897 | 1.00 | 10.72 | A |
| ATOM | 7259 | CA | ASP | A | 927 | 48.430 | 64.415 | −18.928 | 1.00 | 11.43 | A |
| ATOM | 7260 | CB | ASP | A | 927 | 48.847 | 62.968 | −18.666 | 1.00 | 10.70 | A |
| ATOM | 7261 | CG | ASP | A | 927 | 48.500 | 62.520 | −17.260 | 1.00 | 11.69 | A |
| ATOM | 7262 | OD1 | ASP | A | 927 | 49.209 | 62.924 | −16.315 | 1.00 | 10.68 | A |
| ATOM | 7263 | OD2 | ASP | A | 927 | 47.509 | 61.782 | −17.098 | 1.00 | 11.79 | A |
| ATOM | 7264 | C | ASP | A | 927 | 49.022 | 64.910 | −20.238 | 1.00 | 10.90 | A |
| ATOM | 7265 | O | ASP | A | 927 | 49.586 | 64.141 | −21.015 | 1.00 | 10.70 | A |
| ATOM | 7266 | N | PRO | A | 928 | 48.900 | 66.220 | −20.493 | 1.00 | 11.75 | A |
| ATOM | 7267 | CD | PRO | A | 928 | 48.353 | 67.244 | −19.583 | 1.00 | 11.72 | A |
| ATOM | 7268 | CA | PRO | A | 928 | 49.418 | 66.837 | −21.714 | 1.00 | 11.93 | A |
| ATOM | 7269 | CB | PRO | A | 928 | 48.776 | 68.218 | −21.688 | 1.00 | 12.47 | A |
| ATOM | 7270 | CG | PRO | A | 928 | 48.823 | 68.550 | −20.232 | 1.00 | 12.77 | A |
| ATOM | 7271 | C | PRO | A | 928 | 50.932 | 66.928 | −21.666 | 1.00 | 11.95 | A |
| ATOM | 7272 | O | PRO | A | 928 | 51.550 | 66.661 | −20.632 | 1.00 | 12.15 | A |
| ATOM | 7273 | N | LEU | A | 929 | 51.531 | 67.305 | −22.790 | 1.00 | 11.74 | A |
| ATOM | 7274 | CA | LEU | A | 929 | 52.972 | 67.475 | −22.829 | 1.00 | 11.78 | A |
| ATOM | 7275 | CB | LEU | A | 929 | 53.441 | 67.845 | −24.238 | 1.00 | 11.20 | A |
| ATOM | 7276 | CG | LEU | A | 929 | 53.239 | 66.863 | −25.387 | 1.00 | 10.78 | A |
| ATOM | 7277 | CD1 | LEU | A | 929 | 53.803 | 67.478 | −26.667 | 1.00 | 10.05 | A |
| ATOM | 7278 | CD2 | LEU | A | 929 | 53.945 | 65.545 | −25.076 | 1.00 | 10.07 | A |
| ATOM | 7279 | C | LEU | A | 929 | 53.282 | 68.645 | −21.904 | 1.00 | 11.69 | A |
| ATOM | 7280 | O | LEU | A | 929 | 52.479 | 69.568 | −21.778 | 1.00 | 12.26 | A |
| ATOM | 7281 | N | ASP | A | 930 | 54.433 | 68.598 | −21.245 | 1.00 | 11.94 | A |
| ATOM | 7282 | CA | ASP | A | 930 | 54.852 | 69.694 | −20.381 | 1.00 | 12.26 | A |
| ATOM | 7283 | CB | ASP | A | 930 | 55.596 | 69.151 | −19.167 | 1.00 | 11.95 | A |
| ATOM | 7284 | CG | ASP | A | 930 | 54.766 | 68.167 | −18.392 | 1.00 | 12.69 | A |
| ATOM | 7285 | OD1 | ASP | A | 930 | 53.734 | 68.594 | −17.835 | 1.00 | 12.39 | A |
| ATOM | 7286 | OD2 | ASP | A | 930 | 55.134 | 66.973 | −18.357 | 1.00 | 11.41 | A |
| ATOM | 7287 | C | ASP | A | 930 | 55.778 | 70.558 | −21.227 | 1.00 | 12.84 | A |
| ATOM | 7288 | O | ASP | A | 930 | 56.532 | 70.039 | −22.052 | 1.00 | 13.10 | A |
| ATOM | 7289 | N | LYS | A | 931 | 55.723 | 71.870 | −21.025 | 1.00 | 12.78 | A |
| ATOM | 7290 | CA | LYS | A | 931 | 56.542 | 72.789 | −21.807 | 1.00 | 13.16 | A |
| ATOM | 7291 | CB | LYS | A | 931 | 55.630 | 73.733 | −22.600 | 1.00 | 13.75 | A |
| ATOM | 7292 | CG | LYS | A | 931 | 54.632 | 73.015 | −23.505 | 1.00 | 13.74 | A |
| ATOM | 7293 | CD | LYS | A | 931 | 53.724 | 74.003 | −24.235 | 1.00 | 15.47 | A |
| ATOM | 7294 | CE | LYS | A | 931 | 52.865 | 74.794 | −23.260 | 1.00 | 16.58 | A |
| ATOM | 7295 | NZ | LYS | A | 931 | 51.992 | 75.781 | −23.944 | 1.00 | 16.56 | A |
| ATOM | 7296 | C | LYS | A | 931 | 57.512 | 73.605 | −20.964 | 1.00 | 13.32 | A |
| ATOM | 7297 | O | LYS | A | 931 | 57.118 | 74.237 | −19.985 | 1.00 | 13.84 | A |
| ATOM | 7298 | N | PHE | A | 932 | 58.780 | 73.606 | −21.371 | 1.00 | 13.15 | A |
| ATOM | 7299 | CA | PHE | A | 932 | 59.822 | 74.337 | −20.659 | 1.00 | 12.96 | A |
| ATOM | 7300 | CB | PHE | A | 932 | 60.893 | 73.374 | −20.136 | 1.00 | 12.56 | A |
| ATOM | 7301 | CG | PHE | A | 932 | 60.359 | 72.279 | −19.260 | 1.00 | 13.12 | A |
| ATOM | 7302 | CD1 | PHE | A | 932 | 59.737 | 71.165 | −19.814 | 1.00 | 13.77 | A |
| ATOM | 7303 | CD2 | PHE | A | 932 | 60.501 | 72.351 | −17.877 | 1.00 | 14.17 | A |
| ATOM | 7304 | CE1 | PHE | A | 932 | 59.266 | 70.129 | −19.002 | 1.00 | 14.15 | A |
| ATOM | 7305 | CE2 | PHE | A | 932 | 60.035 | 71.327 | −17.056 | 1.00 | 14.64 | A |
| ATOM | 7306 | CZ | PHE | A | 932 | 59.416 | 70.213 | −17.619 | 1.00 | 14.94 | A |
| ATOM | 7307 | C | PHE | A | 932 | 60.509 | 75.378 | −21.544 | 1.00 | 12.75 | A |
| ATOM | 7308 | O | PHE | A | 932 | 60.875 | 75.090 | −22.678 | 1.00 | 12.07 | A |
| ATOM | 7309 | N | ILE | A | 933 | 60.674 | 76.589 | −21.018 | 1.00 | 12.97 | A |
| ATOM | 7310 | CA | ILE | A | 933 | 61.343 | 77.668 | −21.750 | 1.00 | 12.57 | A |
| ATOM | 7311 | CB | ILE | A | 933 | 60.542 | 78.986 | −21.685 | 1.00 | 12.77 | A |
| ATOM | 7312 | CG2 | ILE | A | 933 | 61.264 | 80.066 | −22.486 | 1.00 | 11.64 | A |
| ATOM | 7313 | CG1 | ILE | A | 933 | 59.118 | 78.771 | −22.204 | 1.00 | 12.09 | A |
| ATOM | 7314 | CD1 | ILE | A | 933 | 58.212 | 79.983 | −22.024 | 1.00 | 12.14 | A |
| ATOM | 7315 | C | ILE | A | 933 | 62.696 | 77.921 | −21.084 | 1.00 | 13.11 | A |
| ATOM | 7316 | O | ILE | A | 933 | 62.749 | 78.206 | −19.888 | 1.00 | 13.09 | A |
| ATOM | 7317 | N | PHE | A | 934 | 63.786 | 77.816 | −21.842 | 1.00 | 13.10 | A |
| ATOM | 7318 | CA | PHE | A | 934 | 65.107 | 78.047 | −21.265 | 1.00 | 14.56 | A |
| ATOM | 7319 | CB | PHE | A | 934 | 66.205 | 77.743 | −22.286 | 1.00 | 14.68 | A |
| ATOM | 7320 | CG | PHE | A | 934 | 67.580 | 77.722 | −21.690 | 1.00 | 15.61 | A |
| ATOM | 7321 | CD1 | PHE | A | 934 | 67.960 | 76.697 | −20.828 | 1.00 | 15.02 | A |
| ATOM | 7322 | CD2 | PHE | A | 934 | 68.481 | 78.750 | −21.952 | 1.00 | 15.60 | A |
| ATOM | 7323 | CE1 | PHE | A | 934 | 69.220 | 76.695 | −20.227 | 1.00 | 16.49 | A |
| ATOM | 7324 | CE2 | PHE | A | 934 | 69.742 | 78.761 | −21.360 | 1.00 | 16.52 | A |
| ATOM | 7325 | CZ | PHE | A | 934 | 70.113 | 77.730 | −20.494 | 1.00 | 16.83 | A |
| ATOM | 7326 | C | PHE | A | 934 | 65.202 | 79.506 | −20.808 | 1.00 | 15.07 | A |
| ATOM | 7327 | O | PHE | A | 934 | 64.926 | 80.422 | −21.581 | 1.00 | 14.45 | A |
| ATOM | 7328 | N | ALA | A | 935 | 65.597 | 79.714 | −19.554 | 1.00 | 16.05 | A |
| ATOM | 7329 | CA | ALA | A | 935 | 65.682 | 81.057 | −18.978 | 1.00 | 17.74 | A |
| ATOM | 7330 | CB | ALA | A | 935 | 65.734 | 80.957 | −17.458 | 1.00 | 18.05 | A |
| ATOM | 7331 | C | ALA | A | 935 | 66.825 | 81.946 | −19.468 | 1.00 | 19.42 | A |
| ATOM | 7332 | O | ALA | A | 935 | 66.605 | 83.113 | −19.805 | 1.00 | 20.11 | A |
| ATOM | 7333 | N | GLU | A | 936 | 68.038 | 81.404 | −19.500 | 1.00 | 19.30 | A |
| ATOM | 7334 | CA | GLU | A | 936 | 69.213 | 82.163 | −19.927 | 1.00 | 20.81 | A |
| ATOM | 7335 | CB | GLU | A | 936 | 70.488 | 81.439 | −19.485 | 1.00 | 21.98 | A |
| ATOM | 7336 | CG | GLU | A | 936 | 70.651 | 81.309 | −17.975 | 1.00 | 24.47 | A |
| ATOM | 7337 | CD | GLU | A | 936 | 71.765 | 80.343 | −17.592 | 1.00 | 26.26 | A |
| ATOM | 7338 | OE1 | GLU | A | 936 | 71.580 | 79.117 | −17.761 | 1.00 | 26.72 | A |
| ATOM | 7339 | OE2 | GLU | A | 936 | 72.829 | 80.807 | −17.131 | 1.00 | 27.77 | A |
| ATOM | 7340 | C | GLU | A | 936 | 69.252 | 82.390 | −21.437 | 1.00 | 20.69 | A |
| ATOM | 7341 | O | GLU | A | 936 | 68.439 | 81.841 | −22.180 | 1.00 | 20.44 | A |
| ATOM | 7342 | N | ASN | A | 937 | 70.204 | 83.199 | −21.889 | 1.00 | 20.78 | A |
| ATOM | 7343 | CA | ASN | A | 937 | 70.318 | 83.485 | −23.311 | 1.00 | 21.36 | A |
| ATOM | 7344 | CB | ASN | A | 937 | 71.173 | 84.735 | −23.544 | 1.00 | 22.75 | A |
| ATOM | 7345 | CG | ASN | A | 937 | 70.505 | 85.996 | −23.033 | 1.00 | 24.01 | A |
| ATOM | 7346 | OD1 | ASN | A | 937 | 69.288 | 86.162 | −23.152 | 1.00 | 23.86 | A |
| ATOM | 7347 | ND2 | ASN | A | 937 | 71.298 | 86.900 | −22.477 | 1.00 | 25.45 | A |
| ATOM | 7348 | C | ASN | A | 937 | 70.888 | 82.316 | −24.101 | 1.00 | 21.20 | A |
| ATOM | 7349 | O | ASN | A | 937 | 70.477 | 82.080 | −25.236 | 1.00 | 21.22 | A |
| ATOM | 7350 | N | GLU | A | 938 | 71.827 | 81.585 | −23.507 | 1.00 | 21.16 | A |
| ATOM | 7351 | CA | GLU | A | 938 | 72.426 | 80.442 | −24.189 | 1.00 | 22.06 | A |
| ATOM | 7352 | CB | GLU | A | 938 | 73.828 | 80.795 | −24.700 | 1.00 | 23.73 | A |
| ATOM | 7353 | CG | GLU | A | 938 | 74.576 | 79.599 | −25.276 | 1.00 | 26.61 | A |
| ATOM | 7354 | CD | GLU | A | 938 | 75.794 | 79.992 | −26.085 | 1.00 | 29.03 | A |
| ATOM | 7355 | OE1 | GLU | A | 938 | 76.633 | 80.761 | −25.569 | 1.00 | 30.78 | A |
| ATOM | 7356 | OE2 | GLU | A | 938 | 75.913 | 79.522 | −27.238 | 1.00 | 30.10 | A |
| ATOM | 7357 | C | GLU | A | 938 | 72.501 | 79.184 | −23.327 | 1.00 | 21.20 | A |
| ATOM | 7358 | O | GLU | A | 938 | 72.890 | 79.233 | −22.159 | 1.00 | 21.04 | A |
| ATOM | 7359 | N | TRP | A | 939 | 72.122 | 78.058 | −23.923 | 1.00 | 21.05 | A |
| ATOM | 7360 | CA | TRP | A | 939 | 72.140 | 76.770 | −23.240 | 1.00 | 20.83 | A |
| ATOM | 7361 | CB | TRP | A | 939 | 70.826 | 76.026 | −23.509 | 1.00 | 19.37 | A |
| ATOM | 7362 | CG | TRP | A | 939 | 70.706 | 74.673 | −22.853 | 1.00 | 17.33 | A |
| ATOM | 7363 | CD2 | TRP | A | 939 | 69.626 | 73.743 | −23.011 | 1.00 | 16.09 | A |
| ATOM | 7364 | CE2 | TRP | A | 939 | 69.929 | 72.610 | −22.225 | 1.00 | 16.09 | A |
| ATOM | 7365 | CE3 | TRP | A | 939 | 68.432 | 73.759 | −23.743 | 1.00 | 15.61 | A |
| ATOM | 7366 | CD1 | TRP | A | 939 | 71.596 | 74.082 | −22.000 | 1.00 | 17.03 | A |
| ATOM | 7367 | NE1 | TRP | A | 939 | 71.137 | 72.841 | −21.618 | 1.00 | 16.57 | A |
| ATOM | 7368 | CZ2 | TRP | A | 939 | 69.079 | 71.501 | −22.150 | 1.00 | 15.03 | A |
| ATOM | 7369 | CZ3 | TRP | A | 939 | 67.585 | 72.655 | −23.669 | 1.00 | 15.11 | A |
| ATOM | 7370 | CH2 | TRP | A | 939 | 67.915 | 71.543 | −22.876 | 1.00 | 14.62 | A |
| ATOM | 7371 | C | TRP | A | 939 | 73.329 | 75.971 | −23.764 | 1.00 | 21.47 | A |
| ATOM | 7372 | O | TRP | A | 939 | 73.244 | 75.312 | −24.799 | 1.00 | 21.83 | A |
| ATOM | 7373 | N | ILE | A | 940 | 74.442 | 76.044 | −23.047 | 1.00 | 22.34 | A |
| ATOM | 7374 | CA | ILE | A | 940 | 75.652 | 75.337 | −23.449 | 1.00 | 23.09 | A |
| ATOM | 7375 | CB | ILE | A | 940 | 76.883 | 75.915 | −22.715 | 1.00 | 23.89 | A |
| ATOM | 7376 | CG2 | ILE | A | 940 | 78.139 | 75.154 | −23.108 | 1.00 | 24.48 | A |
| ATOM | 7377 | CG1 | ILE | A | 940 | 77.034 | 77.399 | −23.064 | 1.00 | 24.81 | A |
| ATOM | 7378 | CD1 | ILE | A | 940 | 78.176 | 78.092 | −22.348 | 1.00 | 25.61 | A |
| ATOM | 7379 | C | ILE | A | 940 | 75.548 | 73.836 | −23.178 | 1.00 | 22.56 | A |
| ATOM | 7380 | O | ILE | A | 940 | 75.178 | 73.418 | −22.083 | 1.00 | 23.32 | A |
| ATOM | 7381 | N | GLY | A | 941 | 75.865 | 73.032 | −24.188 | 1.00 | 22.27 | A |
| ATOM | 7382 | CA | GLY | A | 941 | 75.806 | 71.589 | −24.034 | 1.00 | 21.71 | A |
| ATOM | 7383 | C | GLY | A | 941 | 74.420 | 71.002 | −24.242 | 1.00 | 21.38 | A |
| ATOM | 7384 | O | GLY | A | 941 | 74.192 | 69.822 | −23.964 | 1.00 | 20.47 | A |
| ATOM | 7385 | N | ALA | A | 942 | 73.497 | 71.819 | −24.744 | 1.00 | 20.82 | A |
| ATOM | 7386 | CA | ALA | A | 942 | 72.127 | 71.379 | −24.990 | 1.00 | 20.86 | A |
| ATOM | 7387 | CB | ALA | A | 942 | 71.307 | 72.534 | −25.561 | 1.00 | 20.40 | A |
| ATOM | 7388 | C | ALA | A | 942 | 72.023 | 70.170 | −25.920 | 1.00 | 20.97 | A |
| ATOM | 7389 | O | ALA | A | 942 | 72.756 | 70.062 | −26.904 | 1.00 | 20.70 | A |
| ATOM | 7390 | N | GLN | A | 943 | 71.101 | 69.265 | −25.600 | 1.00 | 20.56 | A |
| ATOM | 7391 | CA | GLN | A | 943 | 70.868 | 68.071 | −26.409 | 1.00 | 20.90 | A |
| ATOM | 7392 | CB | GLN | A | 943 | 71.085 | 66.807 | −25.575 | 1.00 | 21.74 | A |
| ATOM | 7393 | CG | GLN | A | 943 | 72.449 | 66.764 | −24.900 | 1.00 | 24.32 | A |
| ATOM | 7394 | CD | GLN | A | 943 | 72.713 | 65.453 | −24.187 | 1.00 | 26.18 | A |
| ATOM | 7395 | OE1 | GLN | A | 943 | 71.852 | 64.939 | −23.469 | 1.00 | 27.65 | A |
| ATOM | 7396 | NE2 | GLN | A | 943 | 73.912 | 64.908 | −24.373 | 1.00 | 26.08 | A |
| ATOM | 7397 | C | GLN | A | 943 | 69.437 | 68.133 | −26.936 | 1.00 | 20.33 | A |
| ATOM | 7398 | O | GLN | A | 943 | 68.570 | 68.759 | −26.325 | 1.00 | 19.79 | A |
| ATOM | 7399 | N | GLY | A | 944 | 69.184 | 67.475 | −28.061 | 1.00 | 19.43 | A |
| ATOM | 7400 | CA | GLY | A | 944 | 67.862 | 67.544 | −28.656 | 1.00 | 19.33 | A |
| ATOM | 7401 | C | GLY | A | 944 | 66.799 | 66.552 | −28.246 | 1.00 | 19.13 | A |
| ATOM | 7402 | O | GLY | A | 944 | 65.624 | 66.761 | −28.546 | 1.00 | 18.88 | A |
| ATOM | 7403 | N | GLN | A | 945 | 67.179 | 65.484 | −27.557 | 1.00 | 18.55 | A |
| ATOM | 7404 | CA | GLN | A | 945 | 66.181 | 64.493 | −27.185 | 1.00 | 18.84 | A |
| ATOM | 7405 | CB | GLN | A | 945 | 65.800 | 63.702 | −28.438 | 1.00 | 20.33 | A |
| ATOM | 7406 | CG | GLN | A | 945 | 64.869 | 62.523 | −28.233 | 1.00 | 22.92 | A |
| ATOM | 7407 | CD | GLN | A | 945 | 64.602 | 61.784 | −29.534 | 1.00 | 24.47 | A |
| ATOM | 7408 | OE1 | GLN | A | 945 | 63.852 | 62.257 | −30.393 | 1.00 | 25.99 | A |
| ATOM | 7409 | NE2 | GLN | A | 945 | 65.231 | 60.627 | −29.693 | 1.00 | 25.24 | A |
| ATOM | 7410 | C | GLN | A | 945 | 66.631 | 63.539 | −26.085 | 1.00 | 18.08 | A |
| ATOM | 7411 | O | GLN | A | 945 | 67.823 | 63.289 | −25.907 | 1.00 | 17.45 | A |
| ATOM | 7412 | N | PHE | A | 946 | 65.653 | 63.023 | −25.348 | 1.00 | 16.69 | A |
| ATOM | 7413 | CA | PHE | A | 946 | 65.890 | 62.056 | −24.288 | 1.00 | 16.10 | A |
| ATOM | 7414 | CB | PHE | A | 946 | 65.869 | 62.717 | −22.906 | 1.00 | 16.05 | A |
| ATOM | 7415 | CG | PHE | A | 946 | 65.756 | 61.730 | −21.774 | 1.00 | 16.43 | A |
| ATOM | 7416 | CD1 | PHE | A | 946 | 66.783 | 60.826 | −21.518 | 1.00 | 16.58 | A |
| ATOM | 7417 | CD2 | PHE | A | 946 | 64.595 | 61.659 | −21.008 | 1.00 | 16.91 | A |
| ATOM | 7418 | CE1 | PHE | A | 946 | 66.657 | 59.854 | −20.514 | 1.00 | 17.50 | A |
| ATOM | 7419 | CE2 | PHE | A | 946 | 64.454 | 60.696 | −20.003 | 1.00 | 17.48 | A |
| ATOM | 7420 | CZ | PHE | A | 946 | 65.489 | 59.788 | −19.755 | 1.00 | 17.10 | A |
| ATOM | 7421 | C | PHE | A | 946 | 64.793 | 61.004 | −24.345 | 1.00 | 15.93 | A |
| ATOM | 7422 | O | PHE | A | 946 | 63.613 | 61.334 | −24.472 | 1.00 | 15.13 | A |
| ATOM | 7423 | N | GLY | A | 947 | 65.189 | 59.737 | −24.256 | 1.00 | 16.02 | A |
| ATOM | 7424 | CA | GLY | A | 947 | 64.226 | 58.653 | −24.275 | 1.00 | 15.95 | A |
| ATOM | 7425 | C | GLY | A | 947 | 63.868 | 58.094 | −25.635 | 1.00 | 16.90 | A |
| ATOM | 7426 | O | GLY | A | 947 | 62.901 | 57.345 | −25.758 | 1.00 | 16.77 | A |
| ATOM | 7427 | N | GLY | A | 948 | 64.634 | 58.450 | −26.661 | 1.00 | 17.57 | A |
| ATOM | 7428 | CA | GLY | A | 948 | 64.347 | 57.938 | −27.987 | 1.00 | 18.79 | A |
| ATOM | 7429 | C | GLY | A | 948 | 64.376 | 56.422 | −28.010 | 1.00 | 19.63 | A |
| ATOM | 7430 | O | GLY | A | 948 | 63.774 | 55.793 | −28.879 | 1.00 | 20.35 | A |
| ATOM | 7431 | N | ASP | A | 949 | 65.070 | 55.831 | −27.043 | 1.00 | 20.59 | A |
| ATOM | 7432 | CA | ASP | A | 949 | 65.182 | 54.381 | −26.961 | 1.00 | 21.92 | A |
| ATOM | 7433 | CB | ASP | A | 949 | 66.603 | 53.991 | −26.538 | 1.00 | 23.50 | A |
| ATOM | 7434 | CG | ASP | A | 949 | 66.942 | 54.453 | −25.134 | 1.00 | 25.82 | A |
| ATOM | 7435 | OD1 | ASP | A | 949 | 66.489 | 55.550 | −24.737 | 1.00 | 26.87 | A |
| ATOM | 7436 | OD2 | ASP | A | 949 | 67.673 | 53.726 | −24.429 | 1.00 | 28.01 | A |
| ATOM | 7437 | C | ASP | A | 949 | 64.164 | 53.755 | −26.007 | 1.00 | 21.81 | A |
| ATOM | 7438 | O | ASP | A | 949 | 64.149 | 52.535 | −25.829 | 1.00 | 22.12 | A |
| ATOM | 7439 | N | HIS | A | 950 | 63.320 | 54.579 | −25.387 | 1.00 | 20.91 | A |
| ATOM | 7440 | CA | HIS | A | 950 | 62.304 | 54.051 | −24.476 | 1.00 | 20.31 | A |
| ATOM | 7441 | CB | HIS | A | 950 | 61.564 | 55.173 | −23.741 | 1.00 | 19.60 | A |
| ATOM | 7442 | CG | HIS | A | 950 | 62.390 | 55.898 | −22.725 | 1.00 | 18.82 | A |
| ATOM | 7443 | CD2 | HIS | A | 950 | 62.137 | 57.027 | −22.023 | 1.00 | 17.61 | A |
| ATOM | 7444 | ND1 | HIS | A | 950 | 63.626 | 55.457 | −22.308 | 1.00 | 19.62 | A |
| ATOM | 7445 | CE1 | HIS | A | 950 | 64.101 | 56.285 | −21.394 | 1.00 | 18.39 | A |
| ATOM | 7446 | NE2 | HIS | A | 950 | 63.216 | 57.245 | −21.203 | 1.00 | 19.83 | A |
| ATOM | 7447 | C | HIS | A | 950 | 61.279 | 53.276 | −25.294 | 1.00 | 20.04 | A |
| ATOM | 7448 | O | HIS | A | 950 | 60.911 | 53.693 | −26.389 | 1.00 | 20.31 | A |
| ATOM | 7449 | N | PRO | A | 951 | 60.806 | 52.133 | −24.776 | 1.00 | 20.23 | A |
| ATOM | 7450 | CD | PRO | A | 951 | 61.345 | 51.371 | −23.634 | 1.00 | 20.95 | A |
| ATOM | 7451 | CA | PRO | A | 951 | 59.813 | 51.343 | −25.507 | 1.00 | 19.76 | A |
| ATOM | 7452 | CB | PRO | A | 951 | 59.683 | 50.081 | −24.655 | 1.00 | 20.56 | A |
| ATOM | 7453 | CG | PRO | A | 951 | 61.048 | 49.953 | −24.038 | 1.00 | 20.81 | A |
| ATOM | 7454 | C | PRO | A | 951 | 58.488 | 52.100 | −25.622 | 1.00 | 19.07 | A |
| ATOM | 7455 | O | PRO | A | 951 | 58.066 | 52.771 | −24.679 | 1.00 | 18.22 | A |
| ATOM | 7456 | N | SER | A | 952 | 57.842 | 52.003 | −26.780 | 1.00 | 18.29 | A |
| ATOM | 7457 | CA | SER | A | 952 | 56.561 | 52.670 | −26.996 | 1.00 | 18.67 | A |
| ATOM | 7458 | CB | SER | A | 952 | 56.487 | 53.235 | −28.418 | 1.00 | 18.74 | A |
| ATOM | 7459 | OG | SER | A | 952 | 55.403 | 54.137 | −28.564 | 1.00 | 18.56 | A |
| ATOM | 7460 | C | SER | A | 952 | 55.493 | 51.605 | −26.791 | 1.00 | 19.01 | A |
| ATOM | 7461 | O | SER | A | 952 | 55.198 | 50.824 | −27.701 | 1.00 | 19.67 | A |
| ATOM | 7462 | N | ALA | A | 953 | 54.920 | 51.581 | −25.589 | 1.00 | 18.23 | A |
| ATOM | 7463 | CA | ALA | A | 953 | 53.916 | 50.591 | −25.214 | 1.00 | 17.70 | A |
| ATOM | 7464 | CB | ALA | A | 953 | 53.744 | 50.589 | −23.699 | 1.00 | 16.83 | A |
| ATOM | 7465 | C | ALA | A | 953 | 52.553 | 50.731 | −25.874 | 1.00 | 17.59 | A |
| ATOM | 7466 | O | ALA | A | 953 | 52.178 | 51.804 | −26.349 | 1.00 | 16.84 | A |
| ATOM | 7467 | N | ARG | A | 954 | 51.813 | 49.625 | −25.874 | 1.00 | 17.23 | A |
| ATOM | 7468 | CA | ARG | A | 954 | 50.476 | 49.572 | −26.443 | 1.00 | 18.11 | A |
| ATOM | 7469 | CB | ARG | A | 954 | 49.859 | 48.202 | −26.170 | 1.00 | 21.07 | A |
| ATOM | 7470 | CG | ARG | A | 954 | 48.514 | 47.967 | −26.832 | 1.00 | 25.46 | A |
| ATOM | 7471 | CD | ARG | A | 954 | 48.386 | 46.508 | −27.244 | 1.00 | 29.24 | A |
| ATOM | 7472 | NE | ARG | A | 954 | 46.998 | 46.084 | −27.381 | 1.00 | 32.43 | A |
| ATOM | 7473 | CZ | ARG | A | 954 | 46.192 | 45.838 | −26.355 | 1.00 | 33.96 | A |
| ATOM | 7474 | NH1 | ARG | A | 954 | 46.641 | 45.975 | −25.116 | 1.00 | 35.54 | A |
| ATOM | 7475 | NH2 | ARG | A | 954 | 44.940 | 45.450 | −26.565 | 1.00 | 35.55 | A |
| ATOM | 7476 | C | ARG | A | 954 | 49.641 | 50.689 | −25.821 | 1.00 | 17.03 | A |
| ATOM | 7477 | O | ARG | A | 954 | 49.743 | 50.962 | −24.620 | 1.00 | 15.07 | A |
| ATOM | 7478 | N | GLU | A | 955 | 48.809 | 51.319 | −26.646 | 1.00 | 16.23 | A |
| ATOM | 7479 | CA | GLU | A | 955 | 47.995 | 52.460 | −26.226 | 1.00 | 16.38 | A |
| ATOM | 7480 | CB | GLU | A | 955 | 47.110 | 52.917 | −27.389 | 1.00 | 17.11 | A |
| ATOM | 7481 | CG | GLU | A | 955 | 45.917 | 52.024 | −27.646 | 1.00 | 19.34 | A |
| ATOM | 7482 | CD | GLU | A | 955 | 45.092 | 52.493 | −28.823 | 1.00 | 20.46 | A |
| ATOM | 7483 | OE1 | GLU | A | 955 | 44.961 | 53.721 | −29.014 | 1.00 | 22.05 | A |
| ATOM | 7484 | OE2 | GLU | A | 955 | 44.564 | 51.632 | −29.551 | 1.00 | 21.62 | A |
| ATOM | 7485 | C | GLU | A | 955 | 47.136 | 52.326 | −24.971 | 1.00 | 15.39 | A |
| ATOM | 7486 | O | GLU | A | 955 | 46.846 | 53.328 | −24.324 | 1.00 | 14.75 | A |
| ATOM | 7487 | N | ASP | A | 956 | 46.722 | 51.114 | −24.617 | 1.00 | 15.12 | A |
| ATOM | 7488 | CA | ASP | A | 956 | 45.892 | 50.957 | −23.425 | 1.00 | 15.34 | A |
| ATOM | 7489 | CB | ASP | A | 956 | 44.895 | 49.793 | −23.595 | 1.00 | 15.33 | A |
| ATOM | 7490 | CG | ASP | A | 956 | 45.572 | 48.461 | −23.883 | 1.00 | 16.25 | A |
| ATOM | 7491 | OD1 | ASP | A | 956 | 46.815 | 48.419 | −24.002 | 1.00 | 16.76 | A |
| ATOM | 7492 | OD2 | ASP | A | 956 | 44.847 | 47.446 | −23.995 | 1.00 | 16.97 | A |
| ATOM | 7493 | C | ASP | A | 956 | 46.714 | 50.777 | −22.152 | 1.00 | 14.55 | A |
| ATOM | 7494 | O | ASP | A | 956 | 46.161 | 50.593 | −21.067 | 1.00 | 15.14 | A |
| ATOM | 7495 | N | LEU | A | 957 | 48.035 | 50.847 | −22.279 | 1.00 | 14.22 | A |
| ATOM | 7496 | CA | LEU | A | 957 | 48.907 | 50.701 | −21.119 | 1.00 | 13.40 | A |
| ATOM | 7497 | CB | LEU | A | 957 | 50.043 | 49.719 | −21.421 | 1.00 | 14.46 | A |
| ATOM | 7498 | CG | LEU | A | 957 | 50.898 | 49.274 | −20.228 | 1.00 | 16.61 | A |
| ATOM | 7499 | CD1 | LEU | A | 957 | 50.032 | 48.511 | −19.227 | 1.00 | 17.21 | A |
| ATOM | 7500 | CD2 | LEU | A | 957 | 52.040 | 48.387 | −20.711 | 1.00 | 16.91 | A |
| ATOM | 7501 | C | LEU | A | 957 | 49.500 | 52.048 | −20.724 | 1.00 | 12.80 | A |
| ATOM | 7502 | O | LEU | A | 957 | 49.850 | 52.857 | −21.582 | 1.00 | 12.44 | A |
| ATOM | 7503 | N | ASP | A | 958 | 49.607 | 52.289 | −19.421 | 1.00 | 11.71 | A |
| ATOM | 7504 | CA | ASP | A | 958 | 50.186 | 53.535 | −18.936 | 1.00 | 11.33 | A |
| ATOM | 7505 | CB | ASP | A | 958 | 49.078 | 54.535 | −18.566 | 1.00 | 11.55 | A |
| ATOM | 7506 | CG | ASP | A | 958 | 49.623 | 55.918 | −18.216 | 1.00 | 11.50 | A |
| ATOM | 7507 | OD1 | ASP | A | 958 | 50.712 | 56.269 | −18.710 | 1.00 | 10.43 | A |
| ATOM | 7508 | OD2 | ASP | A | 958 | 48.952 | 56.657 | −17.458 | 1.00 | 11.83 | A |
| ATOM | 7509 | C | ASP | A | 958 | 51.060 | 53.274 | −17.718 | 1.00 | 11.04 | A |
| ATOM | 7510 | O | ASP | A | 958 | 50.828 | 52.322 | −16.972 | 1.00 | 11.67 | A |
| ATOM | 7511 | N | VAL | A | 959 | 52.095 | 54.091 | −17.558 | 1.00 | 10.57 | A |
| ATOM | 7512 | CA | VAL | A | 959 | 52.967 | 54.015 | −16.392 | 1.00 | 10.41 | A |
| ATOM | 7513 | CB | VAL | A | 959 | 54.442 | 54.315 | −16.747 | 1.00 | 11.51 | A |
| ATOM | 7514 | CG1 | VAL | A | 959 | 55.281 | 54.462 | −15.461 | 1.00 | 11.36 | A |
| ATOM | 7515 | CG2 | VAL | A | 959 | 55.003 | 53.187 | −17.605 | 1.00 | 11.45 | A |
| ATOM | 7516 | C | VAL | A | 959 | 52.401 | 55.148 | −15.539 | 1.00 | 10.64 | A |
| ATOM | 7517 | O | VAL | A | 959 | 52.819 | 56.301 | −15.655 | 1.00 | 10.64 | A |
| ATOM | 7518 | N | SER | A | 960 | 51.413 | 54.810 | −14.715 | 1.00 | 10.31 | A |
| ATOM | 7519 | CA | SER | A | 960 | 50.737 | 55.779 | −13.854 | 1.00 | 10.37 | A |
| ATOM | 7520 | CB | SER | A | 960 | 49.642 | 55.072 | −13.048 | 1.00 | 9.61 | A |
| ATOM | 7521 | OG | SER | A | 960 | 48.815 | 54.293 | −13.899 | 1.00 | 10.39 | A |
| ATOM | 7522 | C | SER | A | 960 | 51.687 | 56.485 | −12.899 | 1.00 | 10.61 | A |
| ATOM | 7523 | O | SER | A | 960 | 51.541 | 57.684 | −12.624 | 1.00 | 10.54 | A |
| ATOM | 7524 | N | VAL | A | 961 | 52.649 | 55.731 | −12.386 | 1.00 | 10.80 | A |
| ATOM | 7525 | CA | VAL | A | 961 | 53.624 | 56.267 | −11.449 | 1.00 | 11.36 | A |
| ATOM | 7526 | CB | VAL | A | 961 | 53.239 | 55.948 | −9.971 | 1.00 | 11.55 | A |
| ATOM | 7527 | CG1 | VAL | A | 961 | 54.337 | 56.447 | −9.019 | 1.00 | 12.60 | A |
| ATOM | 7528 | CG2 | VAL | A | 961 | 51.898 | 56.591 | −9.615 | 1.00 | 10.23 | A |
| ATOM | 7529 | C | VAL | A | 961 | 55.008 | 55.681 | −11.680 | 1.00 | 11.90 | A |
| ATOM | 7530 | O | VAL | A | 961 | 55.159 | 54.481 | −11.935 | 1.00 | 11.41 | A |
| ATOM | 7531 | N | MET | A | 962 | 56.008 | 56.555 | −11.615 | 1.00 | 11.23 | A |
| ATOM | 7532 | CA | MET | A | 962 | 57.402 | 56.166 | −11.705 | 1.00 | 11.17 | A |
| ATOM | 7533 | CB | MET | A | 962 | 58.056 | 56.592 | −13.017 | 1.00 | 11.57 | A |
| ATOM | 7534 | CG | MET | A | 962 | 59.531 | 56.213 | −13.042 | 1.00 | 13.15 | A |
| ATOM | 7535 | SD | MET | A | 962 | 60.354 | 56.539 | −14.597 | 1.00 | 12.96 | A |
| ATOM | 7536 | CE | MET | A | 962 | 62.025 | 55.936 | −14.249 | 1.00 | 13.82 | A |
| ATOM | 7537 | C | MET | A | 962 | 58.006 | 56.954 | −10.555 | 1.00 | 11.31 | A |
| ATOM | 7538 | O | MET | A | 962 | 57.985 | 58.186 | −10.561 | 1.00 | 11.39 | A |
| ATOM | 7539 | N | ARG | A | 963 | 58.524 | 56.247 | −9.559 | 1.00 | 11.55 | A |
| ATOM | 7540 | CA | ARG | A | 963 | 59.078 | 56.909 | −8.387 | 1.00 | 11.46 | A |
| ATOM | 7541 | CB | ARG | A | 963 | 58.004 | 56.968 | −7.285 | 1.00 | 12.00 | A |
| ATOM | 7542 | CG | ARG | A | 963 | 58.487 | 57.451 | −5.911 | 1.00 | 12.06 | A |
| ATOM | 7543 | CD | ARG | A | 963 | 57.365 | 57.332 | −4.865 | 1.00 | 12.84 | A |
| ATOM | 7544 | NE | ARG | A | 963 | 56.177 | 58.061 | −5.303 | 1.00 | 11.77 | A |
| ATOM | 7545 | CZ | ARG | A | 963 | 54.932 | 57.600 | −5.231 | 1.00 | 11.37 | A |
| ATOM | 7546 | NH1 | ARG | A | 963 | 54.681 | 56.400 | −4.719 | 1.00 | 10.90 | A |
| ATOM | 7547 | NH2 | ARG | A | 963 | 53.938 | 58.326 | −5.723 | 1.00 | 9.67 | A |
| ATOM | 7548 | C | ARG | A | 963 | 60.304 | 56.203 | −7.846 | 1.00 | 11.53 | A |
| ATOM | 7549 | O | ARG | A | 963 | 60.241 | 55.021 | −7.522 | 1.00 | 11.39 | A |
| ATOM | 7550 | N | ARG | A | 964 | 61.423 | 56.920 | −7.756 | 1.00 | 12.34 | A |
| ATOM | 7551 | CA | ARG | A | 964 | 62.629 | 56.323 | −7.191 | 1.00 | 12.88 | A |
| ATOM | 7552 | CB | ARG | A | 964 | 63.861 | 57.200 | −7.444 | 1.00 | 12.19 | A |
| ATOM | 7553 | CG | ARG | A | 964 | 65.153 | 56.591 | −6.891 | 1.00 | 12.53 | A |
| ATOM | 7554 | CD | ARG | A | 964 | 66.387 | 57.332 | −7.390 | 1.00 | 12.58 | A |
| ATOM | 7555 | NE | ARG | A | 964 | 66.594 | 57.155 | −8.827 | 1.00 | 13.49 | A |
| ATOM | 7556 | CZ | ARG | A | 964 | 67.474 | 56.317 | −9.367 | 1.00 | 14.26 | A |
| ATOM | 7557 | NH1 | ARG | A | 964 | 68.246 | 55.561 | −8.595 | 1.00 | 14.57 | A |
| ATOM | 7558 | NH2 | ARG | A | 964 | 67.597 | 56.245 | −10.686 | 1.00 | 14.48 | A |
| ATOM | 7559 | C | ARG | A | 964 | 62.314 | 56.255 | −5.702 | 1.00 | 12.97 | A |
| ATOM | 7560 | O | ARG | A | 964 | 61.874 | 57.243 | −5.109 | 1.00 | 13.45 | A |
| ATOM | 7561 | N | LEU | A | 965 | 62.531 | 55.089 | −5.104 | 1.00 | 12.82 | A |
| ATOM | 7562 | CA | LEU | A | 965 | 62.205 | 54.871 | −3.697 | 1.00 | 12.51 | A |
| ATOM | 7563 | CB | LEU | A | 965 | 61.550 | 53.494 | −3.547 | 1.00 | 12.65 | A |
| ATOM | 7564 | CG | LEU | A | 965 | 60.338 | 53.224 | −4.448 | 1.00 | 12.27 | A |
| ATOM | 7565 | CD1 | LEU | A | 965 | 59.993 | 51.743 | −4.424 | 1.00 | 11.69 | A |
| ATOM | 7566 | CD2 | LEU | A | 965 | 59.151 | 54.071 | −3.986 | 1.00 | 12.48 | A |
| ATOM | 7567 | C | LEU | A | 965 | 63.382 | 54.975 | −2.732 | 1.00 | 13.34 | A |
| ATOM | 7568 | O | LEU | A | 965 | 63.197 | 54.895 | −1.517 | 1.00 | 12.58 | A |
| ATOM | 7569 | N | THR | A | 966 | 64.583 | 55.160 | −3.271 | 1.00 | 12.89 | A |
| ATOM | 7570 | CA | THR | A | 966 | 65.779 | 55.252 | −2.444 | 1.00 | 14.47 | A |
| ATOM | 7571 | CB | THR | A | 966 | 66.766 | 54.113 | −2.774 | 1.00 | 14.67 | A |
| ATOM | 7572 | OG1 | THR | A | 966 | 66.992 | 54.075 | −4.189 | 1.00 | 15.14 | A |
| ATOM | 7573 | CG2 | THR | A | 966 | 66.214 | 52.770 | −2.315 | 1.00 | 14.12 | A |
| ATOM | 7574 | C | THR | A | 966 | 66.527 | 56.566 | −2.619 | 1.00 | 15.23 | A |
| ATOM | 7575 | O | THR | A | 966 | 66.504 | 57.166 | −3.695 | 1.00 | 14.67 | A |
| ATOM | 7576 | N | LYS | A | 967 | 67.187 | 57.005 | −1.553 | 1.00 | 15.85 | A |
| ATOM | 7577 | CA | LYS | A | 967 | 67.984 | 58.221 | −1.603 | 1.00 | 17.59 | A |
| ATOM | 7578 | CB | LYS | A | 967 | 68.095 | 58.847 | −0.213 | 1.00 | 18.92 | A |
| ATOM | 7579 | CG | LYS | A | 967 | 66.756 | 59.361 | 0.317 | 1.00 | 21.33 | A |
| ATOM | 7580 | CD | LYS | A | 967 | 66.940 | 60.188 | 1.576 | 1.00 | 23.62 | A |
| ATOM | 7581 | CE | LYS | A | 967 | 65.629 | 60.790 | 2.045 | 1.00 | 25.85 | A |
| ATOM | 7582 | NZ | LYS | A | 967 | 65.838 | 61.623 | 3.264 | 1.00 | 28.29 | A |
| ATOM | 7583 | C | LYS | A | 967 | 69.362 | 57.835 | −2.151 | 1.00 | 17.80 | A |
| ATOM | 7584 | O | LYS | A | 967 | 69.672 | 56.649 | −2.267 | 1.00 | 17.70 | A |
| ATOM | 7585 | N | SER | A | 968 | 70.181 | 58.828 | −2.484 | 1.00 | 18.45 | A |
| ATOM | 7586 | CA | SER | A | 968 | 71.495 | 58.576 | −3.074 | 1.00 | 19.54 | A |
| ATOM | 7587 | CB | SER | A | 968 | 72.170 | 59.903 | −3.443 | 1.00 | 19.67 | A |
| ATOM | 7588 | OG | SER | A | 968 | 72.503 | 60.649 | −2.287 | 1.00 | 21.14 | A |
| ATOM | 7589 | C | SER | A | 968 | 72.480 | 57.732 | −2.263 | 1.00 | 20.02 | A |
| ATOM | 7590 | O | SER | A | 968 | 73.350 | 57.084 | −2.841 | 1.00 | 20.37 | A |
| ATOM | 7591 | N | SER | A | 969 | 72.348 | 57.726 | −0.942 | 1.00 | 20.75 | A |
| ATOM | 7592 | CA | SER | A | 969 | 73.271 | 56.961 | −0.100 | 1.00 | 21.87 | A |
| ATOM | 7593 | CB | SER | A | 969 | 73.197 | 57.456 | 1.346 | 1.00 | 22.66 | A |
| ATOM | 7594 | OG | SER | A | 969 | 71.899 | 57.264 | 1.880 | 1.00 | 25.79 | A |
| ATOM | 7595 | C | SER | A | 969 | 73.054 | 55.447 | −0.125 | 1.00 | 21.74 | A |
| ATOM | 7596 | O | SER | A | 969 | 73.905 | 54.688 | 0.346 | 1.00 | 21.39 | A |
| ATOM | 7597 | N | ALA | A | 970 | 71.926 | 55.008 | −0.676 | 1.00 | 21.45 | A |
| ATOM | 7598 | CA | ALA | A | 970 | 71.613 | 53.580 | −0.741 | 1.00 | 21.30 | A |
| ATOM | 7599 | CB | ALA | A | 970 | 70.122 | 53.385 | −1.012 | 1.00 | 21.26 | A |
| ATOM | 7600 | C | ALA | A | 970 | 72.429 | 52.823 | −1.786 | 1.00 | 21.32 | A |
| ATOM | 7601 | O | ALA | A | 970 | 72.369 | 53.132 | −2.979 | 1.00 | 21.38 | A |
| ATOM | 7602 | N | LYS | A | 971 | 73.186 | 51.825 | −1.334 | 1.00 | 21.84 | A |
| ATOM | 7603 | CA | LYS | A | 971 | 74.003 | 51.011 | −2.232 | 1.00 | 21.84 | A |
| ATOM | 7604 | CB | LYS | A | 971 | 74.683 | 49.874 | −1.464 | 1.00 | 23.71 | A |
| ATOM | 7605 | CG | LYS | A | 971 | 76.011 | 50.243 | −0.817 | 1.00 | 26.65 | A |
| ATOM | 7606 | CD | LYS | A | 971 | 76.600 | 49.058 | −0.056 | 1.00 | 27.50 | A |
| ATOM | 7607 | CE | LYS | A | 971 | 76.656 | 47.801 | −0.922 | 1.00 | 28.39 | A |
| ATOM | 7608 | NZ | LYS | A | 971 | 77.431 | 47.996 | −2.177 | 1.00 | 29.71 | A |
| ATOM | 7609 | C | LYS | A | 971 | 73.144 | 50.415 | −3.335 | 1.00 | 21.02 | A |
| ATOM | 7610 | O | LYS | A | 971 | 73.519 | 50.434 | −4.506 | 1.00 | 20.93 | A |
| ATOM | 7611 | N | THR | A | 972 | 71.997 | 49.862 | −2.953 | 1.00 | 19.58 | A |
| ATOM | 7612 | CA | THR | A | 972 | 71.087 | 49.279 | −3.923 | 1.00 | 18.21 | A |
| ATOM | 7613 | CB | THR | A | 972 | 70.526 | 47.917 | −3.436 | 1.00 | 18.71 | A |
| ATOM | 7614 | OG1 | THR | A | 972 | 71.604 | 46.985 | −3.270 | 1.00 | 18.53 | A |
| ATOM | 7615 | CG2 | THR | A | 972 | 69.545 | 47.345 | −4.454 | 1.00 | 18.65 | A |
| ATOM | 7616 | C | THR | A | 972 | 69.936 | 50.252 | −4.157 | 1.00 | 17.31 | A |
| ATOM | 7617 | O | THR | A | 972 | 69.116 | 50.492 | −3.266 | 1.00 | 16.03 | A |
| ATOM | 7618 | N | GLN | A | 973 | 69.895 | 50.830 | −5.352 | 1.00 | 16.37 | A |
| ATOM | 7619 | CA | GLN | A | 973 | 68.842 | 51.772 | −5.699 | 1.00 | 16.12 | A |
| ATOM | 7620 | CB | GLN | A | 973 | 69.288 | 52.673 | −6.854 | 1.00 | 15.55 | A |
| ATOM | 7621 | CG | GLN | A | 973 | 70.386 | 53.651 | −6.475 | 1.00 | 15.07 | A |
| ATOM | 7622 | CD | GLN | A | 973 | 69.945 | 54.648 | −5.422 | 1.00 | 15.76 | A |
| ATOM | 7623 | OE1 | GLN | A | 973 | 70.600 | 54.812 | −4.389 | 1.00 | 17.45 | A |
| ATOM | 7624 | NE2 | GLN | A | 973 | 68.835 | 55.329 | −5.680 | 1.00 | 14.14 | A |
| ATOM | 7625 | C | GLN | A | 973 | 67.572 | 51.030 | −6.086 | 1.00 | 15.69 | A |
| ATOM | 7626 | O | GLN | A | 973 | 67.623 | 49.947 | −6.670 | 1.00 | 15.91 | A |
| ATOM | 7627 | N | ARG | A | 974 | 66.432 | 51.621 | −5.752 | 1.00 | 15.66 | A |
| ATOM | 7628 | CA | ARG | A | 974 | 65.150 | 51.019 | −6.067 | 1.00 | 15.61 | A |
| ATOM | 7629 | CB | ARG | A | 974 | 64.501 | 50.477 | −4.793 | 1.00 | 16.37 | A |
| ATOM | 7630 | CG | ARG | A | 974 | 65.322 | 49.406 | −4.075 | 1.00 | 18.08 | A |
| ATOM | 7631 | CD | ARG | A | 974 | 64.715 | 49.070 | −2.711 | 1.00 | 19.28 | A |
| ATOM | 7632 | NE | ARG | A | 974 | 63.449 | 48.345 | −2.819 | 1.00 | 21.95 | A |
| ATOM | 7633 | CZ | ARG | A | 974 | 62.324 | 48.706 | −2.208 | 1.00 | 22.67 | A |
| ATOM | 7634 | NH1 | ARG | A | 974 | 62.297 | 49.790 | −1.443 | 1.00 | 23.60 | A |
| ATOM | 7635 | NH2 | ARG | A | 974 | 61.225 | 47.978 | −2.353 | 1.00 | 22.90 | A |
| ATOM | 7636 | C | ARG | A | 974 | 64.226 | 52.034 | −6.727 | 1.00 | 14.98 | A |
| ATOM | 7637 | O | ARG | A | 974 | 64.137 | 53.191 | −6.296 | 1.00 | 14.44 | A |
| ATOM | 7638 | N | VAL | A | 975 | 63.547 | 51.594 | −7.780 | 1.00 | 14.27 | A |
| ATOM | 7639 | CA | VAL | A | 975 | 62.615 | 52.449 | −8.500 | 1.00 | 13.77 | A |
| ATOM | 7640 | CB | VAL | A | 975 | 63.149 | 52.808 | −9.908 | 1.00 | 14.20 | A |
| ATOM | 7641 | CG1 | VAL | A | 975 | 62.161 | 53.726 | −10.628 | 1.00 | 13.31 | A |
| ATOM | 7642 | CG2 | VAL | A | 975 | 64.502 | 53.493 | −9.783 | 1.00 | 13.27 | A |
| ATOM | 7643 | C | VAL | A | 975 | 61.295 | 51.706 | −8.624 | 1.00 | 13.49 | A |
| ATOM | 7644 | O | VAL | A | 975 | 61.251 | 50.561 | −9.084 | 1.00 | 13.35 | A |
| ATOM | 7645 | N | GLY | A | 976 | 60.220 | 52.360 | −8.196 | 1.00 | 13.11 | A |
| ATOM | 7646 | CA | GLY | A | 976 | 58.911 | 51.741 | −8.252 | 1.00 | 11.84 | A |
| ATOM | 7647 | C | GLY | A | 976 | 58.063 | 52.232 | −9.406 | 1.00 | 11.62 | A |
| ATOM | 7648 | O | GLY | A | 976 | 58.130 | 53.404 | −9.800 | 1.00 | 11.25 | A |
| ATOM | 7649 | N | TYR | A | 977 | 57.263 | 51.324 | −9.949 | 1.00 | 11.21 | A |
| ATOM | 7650 | CA | TYR | A | 977 | 56.381 | 51.637 | −11.062 | 1.00 | 11.69 | A |
| ATOM | 7651 | CB | TYR | A | 977 | 56.870 | 50.992 | −12.362 | 1.00 | 11.44 | A |
| ATOM | 7652 | CG | TYR | A | 977 | 58.265 | 51.359 | −12.787 | 1.00 | 12.42 | A |
| ATOM | 7653 | CD1 | TYR | A | 977 | 59.372 | 50.674 | −12.286 | 1.00 | 12.45 | A |
| ATOM | 7654 | CE1 | TYR | A | 977 | 60.669 | 51.011 | −12.691 | 1.00 | 12.91 | A |
| ATOM | 7655 | CD2 | TYR | A | 977 | 58.482 | 52.390 | −13.700 | 1.00 | 12.69 | A |
| ATOM | 7656 | CE2 | TYR | A | 977 | 59.764 | 52.735 | −14.108 | 1.00 | 12.73 | A |
| ATOM | 7657 | CZ | TYR | A | 977 | 60.853 | 52.047 | −13.604 | 1.00 | 13.25 | A |
| ATOM | 7658 | OH | TYR | A | 977 | 62.124 | 52.406 | −14.009 | 1.00 | 12.80 | A |
| ATOM | 7659 | C | TYR | A | 977 | 54.981 | 51.110 | −10.823 | 1.00 | 11.50 | A |
| ATOM | 7660 | O | TYR | A | 977 | 54.805 | 49.989 | −10.347 | 1.00 | 11.45 | A |
| ATOM | 7661 | N | VAL | A | 978 | 53.985 | 51.921 | −11.157 | 1.00 | 11.25 | A |
| ATOM | 7662 | CA | VAL | A | 978 | 52.607 | 51.483 | −11.058 | 1.00 | 11.69 | A |
| ATOM | 7663 | CB | VAL | A | 978 | 51.729 | 52.453 | −10.250 | 1.00 | 12.09 | A |
| ATOM | 7664 | CG1 | VAL | A | 978 | 50.267 | 52.034 | −10.360 | 1.00 | 11.83 | A |
| ATOM | 7665 | CG2 | VAL | A | 978 | 52.160 | 52.438 | −8.784 | 1.00 | 11.63 | A |
| ATOM | 7666 | C | VAL | A | 978 | 52.148 | 51.447 | −12.512 | 1.00 | 12.55 | A |
| ATOM | 7667 | O | VAL | A | 978 | 52.174 | 52.463 | −13.209 | 1.00 | 11.76 | A |
| ATOM | 7668 | N | LEU | A | 979 | 51.775 | 50.256 | −12.968 | 1.00 | 12.63 | A |
| ATOM | 7669 | CA | LEU | A | 979 | 51.330 | 50.048 | −14.337 | 1.00 | 14.77 | A |
| ATOM | 7670 | CB | LEU | A | 979 | 52.064 | 48.855 | −14.957 | 1.00 | 16.74 | A |
| ATOM | 7671 | CG | LEU | A | 979 | 53.380 | 49.107 | −15.694 | 1.00 | 19.88 | A |
| ATOM | 7672 | CD1 | LEU | A | 979 | 53.085 | 49.751 | −17.036 | 1.00 | 20.58 | A |
| ATOM | 7673 | CD2 | LEU | A | 979 | 54.293 | 49.986 | −14.851 | 1.00 | 21.07 | A |
| ATOM | 7674 | C | LEU | A | 979 | 49.841 | 49.786 | −14.406 | 1.00 | 14.34 | A |
| ATOM | 7675 | O | LEU | A | 979 | 49.334 | 48.872 | −13.760 | 1.00 | 14.54 | A |
| ATOM | 7676 | N | HIS | A | 980 | 49.140 | 50.587 | −15.196 | 1.00 | 13.95 | A |
| ATOM | 7677 | CA | HIS | A | 980 | 47.713 | 50.388 | −15.347 | 1.00 | 13.39 | A |
| ATOM | 7678 | CB | HIS | A | 980 | 46.922 | 51.586 | −14.823 | 1.00 | 13.91 | A |
| ATOM | 7679 | CG | HIS | A | 980 | 45.444 | 51.434 | −14.999 | 1.00 | 14.48 | A |
| ATOM | 7680 | CD2 | HIS | A | 980 | 44.542 | 50.693 | −14.315 | 1.00 | 13.79 | A |
| ATOM | 7681 | ND1 | HIS | A | 980 | 44.752 | 52.011 | −16.042 | 1.00 | 15.45 | A |
| ATOM | 7682 | CE1 | HIS | A | 980 | 43.488 | 51.631 | −15.993 | 1.00 | 14.19 | A |
| ATOM | 7683 | NE2 | HIS | A | 980 | 43.334 | 50.829 | −14.955 | 1.00 | 15.58 | A |
| ATOM | 7684 | C | HIS | A | 980 | 47.337 | 50.152 | −16.798 | 1.00 | 13.58 | A |
| ATOM | 7685 | O | HIS | A | 980 | 47.783 | 50.871 | −17.689 | 1.00 | 13.07 | A |
| ATOM | 7686 | N | ARG | A | 981 | 46.521 | 49.131 | −17.026 | 1.00 | 13.43 | A |
| ATOM | 7687 | CA | ARG | A | 981 | 46.053 | 48.830 | −18.367 | 1.00 | 14.40 | A |
| ATOM | 7688 | CB | ARG | A | 981 | 46.438 | 47.411 | −18.777 | 1.00 | 16.66 | A |
| ATOM | 7689 | CG | ARG | A | 981 | 46.067 | 47.071 | −20.213 | 1.00 | 20.47 | A |
| ATOM | 7690 | CD | ARG | A | 981 | 46.616 | 45.714 | −20.602 | 1.00 | 22.48 | A |
| ATOM | 7691 | NE | ARG | A | 981 | 46.190 | 45.316 | −21.939 | 1.00 | 25.91 | A |
| ATOM | 7692 | CZ | ARG | A | 981 | 46.447 | 44.124 | −22.475 | 1.00 | 27.69 | A |
| ATOM | 7693 | NH1 | ARG | A | 981 | 47.129 | 43.217 | −21.787 | 1.00 | 27.99 | A |
| ATOM | 7694 | NH2 | ARG | A | 981 | 46.017 | 43.836 | −23.698 | 1.00 | 28.19 | A |
| ATOM | 7695 | C | ARG | A | 981 | 44.541 | 48.973 | −18.351 | 1.00 | 13.60 | A |
| ATOM | 7696 | O | ARG | A | 981 | 43.850 | 48.296 | −17.592 | 1.00 | 14.15 | A |
| ATOM | 7697 | N | THR | A | 982 | 44.031 | 49.881 | −19.170 | 1.00 | 13.91 | A |
| ATOM | 7698 | CA | THR | A | 982 | 42.597 | 50.105 | −19.252 | 1.00 | 13.49 | A |
| ATOM | 7699 | CB | THR | A | 982 | 42.303 | 51.560 | −19.691 | 1.00 | 13.43 | A |
| ATOM | 7700 | OG1 | THR | A | 982 | 40.900 | 51.825 | −19.587 | 1.00 | 13.78 | A |
| ATOM | 7701 | CG2 | THR | A | 982 | 42.764 | 51.791 | −21.128 | 1.00 | 13.65 | A |
| ATOM | 7702 | C | THR | A | 982 | 42.074 | 49.117 | −20.293 | 1.00 | 14.08 | A |
| ATOM | 7703 | O | THR | A | 982 | 42.817 | 48.244 | −20.743 | 1.00 | 13.64 | A |
| ATOM | 7704 | N | ASN | A | 983 | 40.798 | 49.226 | −20.652 | 1.00 | 13.57 | A |
| ATOM | 7705 | CA | ASN | A | 983 | 40.236 | 48.346 | −21.670 | 1.00 | 13.69 | A |
| ATOM | 7706 | CB | ASN | A | 983 | 39.218 | 47.366 | −21.084 | 1.00 | 13.01 | A |
| ATOM | 7707 | CG | ASN | A | 983 | 38.721 | 46.370 | −22.119 | 1.00 | 13.68 | A |
| ATOM | 7708 | OD1 | ASN | A | 983 | 39.472 | 45.510 | −22.573 | 1.00 | 13.48 | A |
| ATOM | 7709 | ND2 | ASN | A | 983 | 37.456 | 46.497 | −22.513 | 1.00 | 13.45 | A |
| ATOM | 7710 | C | ASN | A | 983 | 39.542 | 49.212 | −22.704 | 1.00 | 14.04 | A |
| ATOM | 7711 | O | ASN | A | 983 | 38.623 | 49.957 | −22.378 | 1.00 | 13.01 | A |
| ATOM | 7712 | N | LEU | A | 984 | 39.994 | 49.110 | −23.949 | 1.00 | 14.70 | A |
| ATOM | 7713 | CA | LEU | A | 984 | 39.425 | 49.888 | −25.040 | 1.00 | 16.23 | A |
| ATOM | 7714 | CB | LEU | A | 984 | 40.536 | 50.572 | −25.831 | 1.00 | 16.53 | A |
| ATOM | 7715 | CG | LEU | A | 984 | 41.501 | 51.426 | −25.003 | 1.00 | 17.01 | A |
| ATOM | 7716 | CD1 | LEU | A | 984 | 42.580 | 51.988 | −25.919 | 1.00 | 17.22 | A |
| ATOM | 7717 | CD2 | LEU | A | 984 | 40.735 | 52.542 | −24.297 | 1.00 | 17.01 | A |
| ATOM | 7718 | C | LEU | A | 984 | 38.640 | 48.976 | −25.961 | 1.00 | 17.29 | A |
| ATOM | 7719 | O | LEU | A | 984 | 39.058 | 47.854 | −26.237 | 1.00 | 17.30 | A |
| ATOM | 7720 | N | MET | A | 985 | 37.500 | 49.458 | −26.438 | 1.00 | 18.60 | A |
| ATOM | 7721 | CA | MET | A | 985 | 36.673 | 48.656 | −27.328 | 1.00 | 20.77 | A |
| ATOM | 7722 | CB | MET | A | 985 | 35.347 | 49.356 | −27.614 | 1.00 | 21.25 | A |
| ATOM | 7723 | CG | MET | A | 985 | 34.437 | 49.491 | −26.421 | 1.00 | 21.90 | A |
| ATOM | 7724 | SD | MET | A | 985 | 32.759 | 49.775 | −26.972 | 1.00 | 24.87 | A |
| ATOM | 7725 | CE | MET | A | 985 | 32.919 | 51.381 | −27.747 | 1.00 | 22.32 | A |
| ATOM | 7726 | C | MET | A | 985 | 37.352 | 48.365 | −28.653 | 1.00 | 22.12 | A |
| ATOM | 7727 | O | MET | A | 985 | 38.039 | 49.216 | −29.220 | 1.00 | 22.17 | A |
| ATOM | 7728 | N | GLN | A | 986 | 37.158 | 47.149 | −29.142 | 1.00 | 23.89 | A |
| ATOM | 7729 | CA | GLN | A | 986 | 37.712 | 46.758 | −30.425 | 1.00 | 25.08 | A |
| ATOM | 7730 | CB | GLN | A | 986 | 38.079 | 45.271 | −30.414 | 1.00 | 27.59 | A |
| ATOM | 7731 | CG | GLN | A | 986 | 37.124 | 44.382 | −29.638 | 1.00 | 30.79 | A |
| ATOM | 7732 | CD | GLN | A | 986 | 37.779 | 43.086 | −29.186 | 1.00 | 32.59 | A |
| ATOM | 7733 | OE1 | GLN | A | 986 | 37.146 | 42.246 | −28.543 | 1.00 | 33.26 | A |
| ATOM | 7734 | NE2 | GLN | A | 986 | 39.059 | 42.923 | −29.516 | 1.00 | 33.73 | A |
| ATOM | 7735 | C | GLN | A | 986 | 36.617 | 47.063 | −31.435 | 1.00 | 24.80 | A |
| ATOM | 7736 | O | GLN | A | 986 | 35.529 | 46.483 | −31.386 | 1.00 | 24.42 | A |
| ATOM | 7737 | N | CYS | A | 987 | 36.899 | 48.003 | −32.330 | 1.00 | 23.92 | A |
| ATOM | 7738 | CA | CYS | A | 987 | 35.922 | 48.412 | −33.327 | 1.00 | 24.36 | A |
| ATOM | 7739 | C | CYS | A | 987 | 36.386 | 48.179 | −34.761 | 1.00 | 26.38 | A |
| ATOM | 7740 | O | CYS | A | 987 | 35.896 | 48.827 | −35.689 | 1.00 | 26.19 | A |
| ATOM | 7741 | CB | CYS | A | 987 | 35.583 | 49.888 | −33.138 | 1.00 | 22.23 | A |
| ATOM | 7742 | SG | CYS | A | 987 | 35.117 | 50.372 | −31.445 | 1.00 | 20.59 | A |
| ATOM | 7743 | N | GLY | A | 988 | 37.338 | 47.268 | −34.942 | 1.00 | 28.36 | A |
| ATOM | 7744 | CA | GLY | A | 988 | 37.814 | 46.963 | −36.280 | 1.00 | 31.14 | A |
| ATOM | 7745 | C | GLY | A | 988 | 39.096 | 47.634 | −36.730 | 1.00 | 33.50 | A |
| ATOM | 7746 | O | GLY | A | 988 | 39.502 | 47.474 | −37.882 | 1.00 | 33.42 | A |
| ATOM | 7747 | N | THR | A | 989 | 39.733 | 48.391 | −35.844 | 1.00 | 35.82 | A |
| ATOM | 7748 | CA | THR | A | 989 | 40.981 | 49.060 | −36.192 | 1.00 | 38.40 | A |
| ATOM | 7749 | CB | THR | A | 989 | 41.153 | 50.377 | −35.414 | 1.00 | 38.55 | A |
| ATOM | 7750 | OG1 | THR | A | 989 | 40.093 | 51.277 | −35.759 | 1.00 | 39.22 | A |
| ATOM | 7751 | CG2 | THR | A | 989 | 42.486 | 51.026 | −35.755 | 1.00 | 38.94 | A |
| ATOM | 7752 | C | THR | A | 989 | 42.158 | 48.140 | −35.880 | 1.00 | 40.35 | A |
| ATOM | 7753 | O | THR | A | 989 | 42.344 | 47.718 | −34.738 | 1.00 | 40.39 | A |
| ATOM | 7754 | N | PRO | A | 990 | 42.969 | 47.816 | −36.901 | 1.00 | 42.23 | A |
| ATOM | 7755 | CD | PRO | A | 990 | 42.821 | 48.277 | −38.295 | 1.00 | 42.52 | A |
| ATOM | 7756 | CA | PRO | A | 990 | 44.139 | 46.943 | −36.768 | 1.00 | 43.92 | A |
| ATOM | 7757 | CB | PRO | A | 990 | 44.879 | 47.172 | −38.079 | 1.00 | 43.82 | A |
| ATOM | 7758 | CG | PRO | A | 990 | 43.749 | 47.342 | −39.047 | 1.00 | 43.19 | A |
| ATOM | 7759 | C | PRO | A | 990 | 45.011 | 47.221 | −35.542 | 1.00 | 45.81 | A |
| ATOM | 7760 | O | PRO | A | 990 | 45.322 | 46.301 | −34.781 | 1.00 | 46.16 | A |
| ATOM | 7761 | N | GLU | A | 991 | 45.406 | 48.479 | −35.353 | 1.00 | 47.49 | A |
| ATOM | 7762 | CA | GLU | A | 991 | 46.240 | 48.856 | −34.211 | 1.00 | 49.39 | A |
| ATOM | 7763 | CB | GLU | A | 991 | 45.384 | 48.904 | −32.942 | 1.00 | 50.54 | A |
| ATOM | 7764 | CG | GLU | A | 991 | 44.254 | 49.927 | −33.000 | 1.00 | 52.51 | A |
| ATOM | 7765 | CD | GLU | A | 991 | 43.249 | 49.762 | −31.872 | 1.00 | 53.74 | A |
| ATOM | 7766 | OE1 | GLU | A | 991 | 43.668 | 49.746 | −30.695 | 1.00 | 54.22 | A |
| ATOM | 7767 | OE2 | GLU | A | 991 | 42.037 | 49.649 | −32.163 | 1.00 | 54.31 | A |
| ATOM | 7768 | C | GLU | A | 991 | 47.386 | 47.857 | −34.034 | 1.00 | 49.78 | A |
| ATOM | 7769 | O | GLU | A | 991 | 47.270 | 46.890 | −33.280 | 1.00 | 49.97 | A |
| ATOM | 7770 | N | GLU | A | 992 | 48.495 | 48.103 | −34.725 | 1.00 | 50.30 | A |
| ATOM | 7771 | CA | GLU | A | 992 | 49.640 | 47.202 | −34.666 | 1.00 | 50.80 | A |
| ATOM | 7772 | CB | GLU | A | 992 | 49.945 | 46.680 | −36.073 | 1.00 | 51.80 | A |
| ATOM | 7773 | CG | GLU | A | 992 | 48.776 | 45.982 | −36.751 | 1.00 | 53.22 | A |
| ATOM | 7774 | CD | GLU | A | 992 | 49.063 | 45.644 | −38.204 | 1.00 | 54.07 | A |
| ATOM | 7775 | OE1 | GLU | A | 992 | 50.053 | 44.926 | −38.467 | 1.00 | 54.55 | A |
| ATOM | 7776 | OE2 | GLU | A | 992 | 48.299 | 46.097 | −39.083 | 1.00 | 54.47 | A |
| ATOM | 7777 | C | GLU | A | 992 | 50.913 | 47.810 | −34.077 | 1.00 | 50.44 | A |
| ATOM | 7778 | O | GLU | A | 992 | 50.869 | 48.734 | −33.264 | 1.00 | 50.69 | A |
| ATOM | 7779 | N | HIS | A | 993 | 52.042 | 47.259 | −34.518 | 1.00 | 49.81 | A |
| ATOM | 7780 | CA | HIS | A | 993 | 53.391 | 47.656 | −34.116 | 1.00 | 48.87 | A |
| ATOM | 7781 | CB | HIS | A | 993 | 54.081 | 48.396 | −35.277 | 1.00 | 49.88 | A |
| ATOM | 7782 | CG | HIS | A | 993 | 53.335 | 49.596 | −35.779 | 1.00 | 51.14 | A |
| ATOM | 7783 | CD2 | HIS | A | 993 | 52.287 | 50.283 | −35.264 | 1.00 | 51.72 | A |
| ATOM | 7784 | ND1 | HIS | A | 993 | 53.669 | 50.236 | −36.954 | 1.00 | 51.70 | A |
| ATOM | 7785 | CE1 | HIS | A | 993 | 52.860 | 51.263 | −37.141 | 1.00 | 51.91 | A |
| ATOM | 7786 | NE2 | HIS | A | 993 | 52.012 | 51.315 | −36.130 | 1.00 | 52.16 | A |
| ATOM | 7787 | C | HIS | A | 993 | 53.594 | 48.436 | −32.815 | 1.00 | 47.56 | A |
| ATOM | 7788 | O | HIS | A | 993 | 53.594 | 49.669 | −32.802 | 1.00 | 47.73 | A |
| ATOM | 7789 | N | THR | A | 994 | 53.783 | 47.695 | −31.725 | 1.00 | 45.51 | A |
| ATOM | 7790 | CA | THR | A | 994 | 54.036 | 48.275 | −30.406 | 1.00 | 42.92 | A |
| ATOM | 7791 | CB | THR | A | 994 | 52.744 | 48.419 | −29.568 | 1.00 | 43.20 | A |
| ATOM | 7792 | OG1 | THR | A | 994 | 52.156 | 47.129 | −29.359 | 1.00 | 43.29 | A |
| ATOM | 7793 | CG2 | THR | A | 994 | 51.747 | 49.325 | −30.277 | 1.00 | 42.99 | A |
| ATOM | 7794 | C | THR | A | 994 | 55.006 | 47.354 | −29.667 | 1.00 | 41.17 | A |
| ATOM | 7795 | O | THR | A | 994 | 54.929 | 46.130 | −29.788 | 1.00 | 41.12 | A |
| ATOM | 7796 | N | GLN | A | 995 | 55.920 | 47.945 | −28.905 | 1.00 | 38.56 | A |
| ATOM | 7797 | CA | GLN | A | 995 | 56.913 | 47.173 | −28.171 | 1.00 | 35.93 | A |
| ATOM | 7798 | CB | GLN | A | 995 | 58.205 | 47.974 | −28.040 | 1.00 | 36.57 | A |
| ATOM | 7799 | CG | GLN | A | 995 | 58.663 | 48.637 | −29.320 | 1.00 | 37.34 | A |
| ATOM | 7800 | CD | GLN | A | 995 | 59.782 | 49.622 | −29.072 | 1.00 | 38.00 | A |
| ATOM | 7801 | OE1 | GLN | A | 995 | 60.896 | 49.239 | −28.706 | 1.00 | 38.62 | A |
| ATOM | 7802 | NE2 | GLN | A | 995 | 59.488 | 50.904 | −29.255 | 1.00 | 36.97 | A |
| ATOM | 7803 | C | GLN | A | 995 | 56.442 | 46.789 | −26.777 | 1.00 | 33.93 | A |
| ATOM | 7804 | O | GLN | A | 995 | 55.653 | 47.500 | −26.154 | 1.00 | 33.07 | A |
| ATOM | 7805 | N | LYS | A | 996 | 56.938 | 45.660 | −26.289 | 1.00 | 31.39 | A |
| ATOM | 7806 | CA | LYS | A | 996 | 56.590 | 45.209 | −24.954 | 1.00 | 29.57 | A |
| ATOM | 7807 | CB | LYS | A | 996 | 57.060 | 43.768 | −24.736 | 1.00 | 30.91 | A |
| ATOM | 7808 | CG | LYS | A | 996 | 56.341 | 42.743 | −25.602 | 1.00 | 32.81 | A |
| ATOM | 7809 | CD | LYS | A | 996 | 54.854 | 42.682 | −25.265 | 1.00 | 34.20 | A |
| ATOM | 7810 | CE | LYS | A | 996 | 54.150 | 41.601 | −26.067 | 1.00 | 34.76 | A |
| ATOM | 7811 | NZ | LYS | A | 996 | 54.719 | 40.254 | −25.779 | 1.00 | 35.51 | A |
| ATOM | 7812 | C | LYS | A | 996 | 57.299 | 46.139 | −23.981 | 1.00 | 27.27 | A |
| ATOM | 7813 | O | LYS | A | 996 | 58.473 | 46.462 | −24.167 | 1.00 | 26.17 | A |
| ATOM | 7814 | N | LEU | A | 997 | 56.585 | 46.584 | −22.954 | 1.00 | 25.15 | A |
| ATOM | 7815 | CA | LEU | A | 997 | 57.181 | 47.470 | −21.968 | 1.00 | 23.54 | A |
| ATOM | 7816 | CB | LEU | A | 997 | 56.211 | 48.595 | −21.592 | 1.00 | 23.51 | A |
| ATOM | 7817 | CG | LEU | A | 997 | 56.692 | 49.527 | −20.469 | 1.00 | 23.27 | A |
| ATOM | 7818 | CD1 | LEU | A | 997 | 57.965 | 50.247 | −20.895 | 1.00 | 23.38 | A |
| ATOM | 7819 | CD2 | LEU | A | 997 | 55.599 | 50.530 | −20.132 | 1.00 | 22.41 | A |
| ATOM | 7820 | C | LEU | A | 997 | 57.573 | 46.705 | −20.716 | 1.00 | 22.50 | A |
| ATOM | 7821 | O | LEU | A | 997 | 56.723 | 46.139 | −20.032 | 1.00 | 22.25 | A |
| ATOM | 7822 | N | ASP | A | 998 | 58.871 | 46.683 | −20.433 | 1.00 | 21.36 | A |
| ATOM | 7823 | CA | ASP | A | 998 | 59.398 | 46.018 | −19.249 | 1.00 | 21.23 | A |
| ATOM | 7824 | CB | ASP | A | 998 | 60.477 | 45.001 | −19.635 | 1.00 | 21.93 | A |
| ATOM | 7825 | CG | ASP | A | 998 | 61.146 | 44.373 | −18.426 | 1.00 | 22.40 | A |
| ATOM | 7826 | OD1 | ASP | A | 998 | 62.161 | 43.667 | −18.611 | 1.00 | 22.65 | A |
| ATOM | 7827 | OD2 | ASP | A | 998 | 60.658 | 44.581 | −17.292 | 1.00 | 22.00 | A |
| ATOM | 7828 | C | ASP | A | 998 | 60.009 | 47.121 | −18.396 | 1.00 | 20.26 | A |
| ATOM | 7829 | O | ASP | A | 998 | 61.182 | 47.459 | −18.553 | 1.00 | 19.78 | A |
| ATOM | 7830 | N | VAL | A | 999 | 59.210 | 47.689 | −17.500 | 1.00 | 19.60 | A |
| ATOM | 7831 | CA | VAL | A | 999 | 59.687 | 48.774 | −16.655 | 1.00 | 18.89 | A |
| ATOM | 7832 | CB | VAL | A | 999 | 58.575 | 49.289 | −15.701 | 1.00 | 18.56 | A |
| ATOM | 7833 | CG1 | VAL | A | 999 | 57.399 | 49.796 | −16.515 | 1.00 | 18.40 | A |
| ATOM | 7834 | CG2 | VAL | A | 999 | 58.136 | 48.191 | −14.743 | 1.00 | 17.96 | A |
| ATOM | 7835 | C | VAL | A | 999 | 60.918 | 48.419 | −15.829 | 1.00 | 19.06 | A |
| ATOM | 7836 | O | VAL | A | 999 | 61.682 | 49.303 | −15.441 | 1.00 | 18.30 | A |
| ATOM | 7837 | N | CYS | A | 1000 | 61.130 | 47.136 | −15.563 | 1.00 | 19.10 | A |
| ATOM | 7838 | CA | CYS | A | 1000 | 62.291 | 46.767 | −14.772 | 1.00 | 20.40 | A |
| ATOM | 7839 | C | CYS | A | 1000 | 63.615 | 46.972 | −15.502 | 1.00 | 19.66 | A |
| ATOM | 7840 | O | CYS | A | 1000 | 64.669 | 46.995 | −14.877 | 1.00 | 19.22 | A |
| ATOM | 7841 | CB | CYS | A | 1000 | 62.157 | 45.333 | −14.258 | 1.00 | 21.94 | A |
| ATOM | 7842 | SG | CYS | A | 1000 | 61.388 | 45.277 | −12.599 | 1.00 | 25.12 | A |
| ATOM | 7843 | N | HIS | A | 1001 | 63.566 | 47.136 | −16.821 | 1.00 | 20.20 | A |
| ATOM | 7844 | CA | HIS | A | 1001 | 64.793 | 47.372 | −17.577 | 1.00 | 20.55 | A |
| ATOM | 7845 | CB | HIS | A | 1001 | 64.928 | 46.373 | −18.729 | 1.00 | 20.76 | A |
| ATOM | 7846 | CG | HIS | A | 1001 | 65.456 | 45.039 | −18.305 | 1.00 | 20.70 | A |
| ATOM | 7847 | CD2 | HIS | A | 1001 | 66.715 | 44.542 | −18.298 | 1.00 | 20.88 | A |
| ATOM | 7848 | ND1 | HIS | A | 1001 | 64.656 | 44.060 | −17.756 | 1.00 | 20.78 | A |
| ATOM | 7849 | CE1 | HIS | A | 1001 | 65.399 | 43.019 | −17.427 | 1.00 | 20.46 | A |
| ATOM | 7850 | NE2 | HIS | A | 1001 | 66.653 | 43.285 | −17.745 | 1.00 | 21.10 | A |
| ATOM | 7851 | C | HIS | A | 1001 | 64.903 | 48.800 | −18.109 | 1.00 | 20.63 | A |
| ATOM | 7852 | O | HIS | A | 1001 | 65.752 | 49.092 | −18.953 | 1.00 | 21.06 | A |
| ATOM | 7853 | N | LEU | A | 1002 | 64.048 | 49.693 | −17.617 | 1.00 | 20.58 | A |
| ATOM | 7854 | CA | LEU | A | 1002 | 64.087 | 51.088 | −18.048 | 1.00 | 20.34 | A |
| ATOM | 7855 | CB | LEU | A | 1002 | 62.875 | 51.855 | −17.518 | 1.00 | 20.22 | A |
| ATOM | 7856 | CG | LEU | A | 1002 | 61.592 | 51.670 | −18.332 | 1.00 | 19.58 | A |
| ATOM | 7857 | CD1 | LEU | A | 1002 | 60.440 | 52.384 | −17.647 | 1.00 | 18.77 | A |
| ATOM | 7858 | CD2 | LEU | A | 1002 | 61.801 | 52.212 | −19.741 | 1.00 | 19.29 | A |
| ATOM | 7859 | C | LEU | A | 1002 | 65.371 | 51.737 | −17.557 | 1.00 | 21.19 | A |
| ATOM | 7860 | O | LEU | A | 1002 | 65.920 | 52.622 | −18.212 | 1.00 | 21.36 | A |
| ATOM | 7861 | N | LEU | A | 1003 | 65.840 | 51.298 | −16.393 | 1.00 | 21.04 | A |
| ATOM | 7862 | CA | LEU | A | 1003 | 67.087 | 51.802 | −15.835 | 1.00 | 21.46 | A |
| ATOM | 7863 | CB | LEU | A | 1003 | 66.902 | 52.223 | −14.377 | 1.00 | 22.21 | A |
| ATOM | 7864 | CG | LEU | A | 1003 | 66.045 | 53.476 | −14.175 | 1.00 | 23.70 | A |
| ATOM | 7865 | CD1 | LEU | A | 1003 | 65.966 | 53.806 | −12.703 | 1.00 | 24.51 | A |
| ATOM | 7866 | CD2 | LEU | A | 1003 | 66.650 | 54.644 | −14.938 | 1.00 | 25.02 | A |
| ATOM | 7867 | C | LEU | A | 1003 | 68.111 | 50.677 | −15.944 | 1.00 | 21.41 | A |
| ATOM | 7868 | O | LEU | A | 1003 | 67.774 | 49.503 | −15.789 | 1.00 | 20.58 | A |
| ATOM | 7869 | N | PRO | A | 1004 | 69.376 | 51.021 | −16.220 | 1.00 | 21.54 | A |
| ATOM | 7870 | CD | PRO | A | 1004 | 69.907 | 52.386 | −16.399 | 1.00 | 21.59 | A |
| ATOM | 7871 | CA | PRO | A | 1004 | 70.445 | 50.029 | −16.360 | 1.00 | 21.82 | A |
| ATOM | 7872 | CB | PRO | A | 1004 | 71.565 | 50.837 | −17.005 | 1.00 | 22.03 | A |
| ATOM | 7873 | CG | PRO | A | 1004 | 71.412 | 52.162 | −16.331 | 1.00 | 21.74 | A |
| ATOM | 7874 | C | PRO | A | 1004 | 70.901 | 49.362 | −15.070 | 1.00 | 22.14 | A |
| ATOM | 7875 | O | PRO | A | 1004 | 70.577 | 49.809 | −13.964 | 1.00 | 21.00 | A |
| ATOM | 7876 | N | ASN | A | 1005 | 71.649 | 48.274 | −15.235 | 1.00 | 22.33 | A |
| ATOM | 7877 | CA | ASN | A | 1005 | 72.208 | 47.531 | −14.114 | 1.00 | 22.54 | A |
| ATOM | 7878 | CB | ASN | A | 1005 | 73.152 | 48.442 | −13.333 | 1.00 | 24.42 | A |
| ATOM | 7879 | CG | ASN | A | 1005 | 74.150 | 49.147 | −14.230 | 1.00 | 26.07 | A |
| ATOM | 7880 | OD1 | ASN | A | 1005 | 74.346 | 50.361 | −14.126 | 1.00 | 27.74 | A |
| ATOM | 7881 | ND2 | ASN | A | 1005 | 74.790 | 48.392 | −15.115 | 1.00 | 25.81 | A |
| ATOM | 7882 | C | ASN | A | 1005 | 71.165 | 46.949 | −13.167 | 1.00 | 22.28 | A |
| ATOM | 7883 | O | ASN | A | 1005 | 71.360 | 46.950 | −11.952 | 1.00 | 22.03 | A |
| ATOM | 7884 | N | VAL | A | 1006 | 70.065 | 46.448 | −13.714 | 1.00 | 22.19 | A |
| ATOM | 7885 | CA | VAL | A | 1006 | 69.022 | 45.869 | −12.877 | 1.00 | 22.43 | A |
| ATOM | 7886 | CB | VAL | A | 1006 | 67.735 | 45.579 | −13.690 | 1.00 | 21.96 | A |
| ATOM | 7887 | CG1 | VAL | A | 1006 | 68.026 | 44.599 | −14.810 | 1.00 | 21.98 | A |
| ATOM | 7888 | CG2 | VAL | A | 1006 | 66.649 | 45.035 | −12.768 | 1.00 | 21.52 | A |
| ATOM | 7889 | C | VAL | A | 1006 | 69.537 | 44.580 | −12.239 | 1.00 | 22.76 | A |
| ATOM | 7890 | O | VAL | A | 1006 | 70.095 | 43.718 | −12.920 | 1.00 | 23.29 | A |
| ATOM | 7891 | N | ALA | A | 1007 | 69.361 | 44.465 | −10.927 | 1.00 | 22.95 | A |
| ATOM | 7892 | CA | ALA | A | 1007 | 69.813 | 43.295 | −10.181 | 1.00 | 23.43 | A |
| ATOM | 7893 | CB | ALA | A | 1007 | 70.578 | 43.737 | −8.935 | 1.00 | 23.72 | A |
| ATOM | 7894 | C | ALA | A | 1007 | 68.640 | 42.398 | −9.793 | 1.00 | 23.92 | A |
| ATOM | 7895 | O | ALA | A | 1007 | 68.816 | 41.207 | −9.533 | 1.00 | 23.84 | A |
| ATOM | 7896 | N | ARG | A | 1008 | 67.445 | 42.975 | −9.736 | 1.00 | 23.57 | A |
| ATOM | 7897 | CA | ARG | A | 1008 | 66.251 | 42.204 | −9.413 | 1.00 | 24.00 | A |
| ATOM | 7898 | CB | ARG | A | 1008 | 66.236 | 41.782 | −7.938 | 1.00 | 26.17 | A |
| ATOM | 7899 | CG | ARG | A | 1008 | 66.171 | 42.910 | −6.933 | 1.00 | 29.81 | A |
| ATOM | 7900 | CD | ARG | A | 1008 | 66.045 | 42.358 | −5.513 | 1.00 | 33.34 | A |
| ATOM | 7901 | NE | ARG | A | 1008 | 67.188 | 41.524 | −5.140 | 1.00 | 35.30 | A |
| ATOM | 7902 | CZ | ARG | A | 1008 | 68.436 | 41.969 | −5.011 | 1.00 | 36.73 | A |
| ATOM | 7903 | NH1 | ARG | A | 1008 | 68.718 | 43.249 | −5.222 | 1.00 | 36.58 | A |
| ATOM | 7904 | NH2 | ARG | A | 1008 | 69.409 | 41.129 | −4.676 | 1.00 | 37.44 | A |
| ATOM | 7905 | C | ARG | A | 1008 | 64.983 | 42.973 | −9.748 | 1.00 | 23.14 | A |
| ATOM | 7906 | O | ARG | A | 1008 | 64.981 | 44.206 | −9.811 | 1.00 | 22.18 | A |
| ATOM | 7907 | N | CYS | A | 1009 | 63.910 | 42.225 | −9.979 | 1.00 | 22.25 | A |
| ATOM | 7908 | CA | CYS | A | 1009 | 62.617 | 42.797 | −10.322 | 1.00 | 21.69 | A |
| ATOM | 7909 | C | CYS | A | 1009 | 61.572 | 42.099 | −9.472 | 1.00 | 20.98 | A |
| ATOM | 7910 | O | CYS | A | 1009 | 61.501 | 40.868 | −9.453 | 1.00 | 19.71 | A |
| ATOM | 7911 | CB | CYS | A | 1009 | 62.322 | 42.559 | −11.799 | 1.00 | 23.19 | A |
| ATOM | 7912 | SG | CYS | A | 1009 | 60.805 | 43.356 | −12.416 | 1.00 | 25.61 | A |
| ATOM | 7913 | N | GLU | A | 1010 | 60.759 | 42.881 | −8.771 | 1.00 | 19.93 | A |
| ATOM | 7914 | CA | GLU | A | 1010 | 59.743 | 42.307 | −7.905 | 1.00 | 19.15 | A |
| ATOM | 7915 | CB | GLU | A | 1010 | 60.178 | 42.459 | −6.444 | 1.00 | 20.68 | A |
| ATOM | 7916 | CG | GLU | A | 1010 | 61.488 | 41.748 | −6.110 | 1.00 | 23.17 | A |
| ATOM | 7917 | CD | GLU | A | 1010 | 62.113 | 42.241 | −4.819 | 1.00 | 24.63 | A |
| ATOM | 7918 | OE1 | GLU | A | 1010 | 62.533 | 43.419 | −4.773 | 1.00 | 25.13 | A |
| ATOM | 7919 | OE2 | GLU | A | 1010 | 62.183 | 41.452 | −3.849 | 1.00 | 25.95 | A |
| ATOM | 7920 | C | GLU | A | 1010 | 58.372 | 42.943 | −8.091 | 1.00 | 18.57 | A |
| ATOM | 7921 | O | GLU | A | 1010 | 58.258 | 44.148 | −8.334 | 1.00 | 17.62 | A |
| ATOM | 7922 | N | ARG | A | 1011 | 57.333 | 42.122 | −7.995 | 1.00 | 17.73 | A |
| ATOM | 7923 | CA | ARG | A | 1011 | 55.970 | 42.623 | −8.087 | 1.00 | 17.39 | A |
| ATOM | 7924 | CB | ARG | A | 1011 | 55.009 | 41.537 | −8.571 | 1.00 | 19.53 | A |
| ATOM | 7925 | CG | ARG | A | 1011 | 53.590 | 42.048 | −8.816 | 1.00 | 23.41 | A |
| ATOM | 7926 | CD | ARG | A | 1011 | 52.581 | 40.910 | −8.808 | 1.00 | 27.12 | A |
| ATOM | 7927 | NE | ARG | A | 1011 | 52.897 | 39.879 | −9.790 | 1.00 | 31.70 | A |
| ATOM | 7928 | CZ | ARG | A | 1011 | 52.840 | 40.054 | −11.107 | 1.00 | 33.53 | A |
| ATOM | 7929 | NH1 | ARG | A | 1011 | 52.477 | 41.229 | −11.607 | 1.00 | 35.13 | A |
| ATOM | 7930 | NH2 | ARG | A | 1011 | 53.145 | 39.052 | −11.925 | 1.00 | 34.20 | A |
| ATOM | 7931 | C | ARG | A | 1011 | 55.654 | 42.980 | −6.637 | 1.00 | 16.18 | A |
| ATOM | 7932 | O | ARG | A | 1011 | 55.992 | 42.226 | −5.722 | 1.00 | 15.62 | A |
| ATOM | 7933 | N | THR | A | 1012 | 55.021 | 44.126 | −6.423 | 1.00 | 14.81 | A |
| ATOM | 7934 | CA | THR | A | 1012 | 54.702 | 44.562 | −5.071 | 1.00 | 13.76 | A |
| ATOM | 7935 | CB | THR | A | 1012 | 55.600 | 45.742 | −4.635 | 1.00 | 14.36 | A |
| ATOM | 7936 | OG1 | THR | A | 1012 | 55.252 | 46.904 | −5.404 | 1.00 | 13.90 | A |
| ATOM | 7937 | CG2 | THR | A | 1012 | 57.072 | 45.422 | −4.858 | 1.00 | 12.72 | A |
| ATOM | 7938 | C | THR | A | 1012 | 53.268 | 45.060 | −4.984 | 1.00 | 13.68 | A |
| ATOM | 7939 | O | THR | A | 1012 | 52.568 | 45.170 | −5.989 | 1.00 | 13.39 | A |
| ATOM | 7940 | N | THR | A | 1013 | 52.838 | 45.355 | −3.764 | 1.00 | 12.69 | A |
| ATOM | 7941 | CA | THR | A | 1013 | 51.518 | 45.915 | −3.552 | 1.00 | 12.46 | A |
| ATOM | 7942 | CB | THR | A | 1013 | 51.225 | 46.036 | −2.046 | 1.00 | 12.35 | A |
| ATOM | 7943 | OG1 | THR | A | 1013 | 52.406 | 46.482 | −1.367 | 1.00 | 11.61 | A |
| ATOM | 7944 | CG2 | THR | A | 1013 | 50.807 | 44.676 | −1.472 | 1.00 | 12.82 | A |
| ATOM | 7945 | C | THR | A | 1013 | 51.611 | 47.303 | −4.203 | 1.00 | 12.06 | A |
| ATOM | 7946 | O | THR | A | 1013 | 52.716 | 47.809 | −4.424 | 1.00 | 11.69 | A |
| ATOM | 7947 | N | LEU | A | 1014 | 50.473 | 47.920 | −4.509 | 1.00 | 12.08 | A |
| ATOM | 7948 | CA | LEU | A | 1014 | 50.479 | 49.228 | −5.171 | 1.00 | 11.50 | A |
| ATOM | 7949 | CB | LEU | A | 1014 | 49.051 | 49.659 | −5.504 | 1.00 | 11.09 | A |
| ATOM | 7950 | CG | LEU | A | 1014 | 48.261 | 48.712 | −6.406 | 1.00 | 10.82 | A |
| ATOM | 7951 | CD1 | LEU | A | 1014 | 46.957 | 49.394 | −6.801 | 1.00 | 10.20 | A |
| ATOM | 7952 | CD2 | LEU | A | 1014 | 49.076 | 48.353 | −7.647 | 1.00 | 10.52 | A |
| ATOM | 7953 | C | LEU | A | 1014 | 51.172 | 50.340 | −4.404 | 1.00 | 11.34 | A |
| ATOM | 7954 | O | LEU | A | 1014 | 51.532 | 51.368 | −4.984 | 1.00 | 11.68 | A |
| ATOM | 7955 | N | THR | A | 1015 | 51.360 | 50.133 | −3.105 | 1.00 | 11.33 | A |
| ATOM | 7956 | CA | THR | A | 1015 | 52.019 | 51.106 | −2.240 | 1.00 | 11.07 | A |
| ATOM | 7957 | CB | THR | A | 1015 | 51.500 | 51.004 | −0.800 | 1.00 | 11.10 | A |
| ATOM | 7958 | OG1 | THR | A | 1015 | 51.640 | 49.648 | −0.353 | 1.00 | 11.46 | A |
| ATOM | 7959 | CG2 | THR | A | 1015 | 50.032 | 51.427 | −0.714 | 1.00 | 10.67 | A |
| ATOM | 7960 | C | THR | A | 1015 | 53.528 | 50.866 | −2.193 | 1.00 | 11.04 | A |
| ATOM | 7961 | O | THR | A | 1015 | 54.262 | 51.636 | −1.563 | 1.00 | 11.09 | A |
| ATOM | 7962 | N | PHE | A | 1016 | 53.972 | 49.794 | −2.850 | 1.00 | 11.39 | A |
| ATOM | 7963 | CA | PHE | A | 1016 | 55.388 | 49.408 | −2.897 | 1.00 | 12.27 | A |
| ATOM | 7964 | CB | PHE | A | 1016 | 56.271 | 50.604 | −3.287 | 1.00 | 12.26 | A |
| ATOM | 7965 | CG | PHE | A | 1016 | 55.966 | 51.184 | −4.639 | 1.00 | 11.88 | A |
| ATOM | 7966 | CD1 | PHE | A | 1016 | 55.835 | 52.562 | −4.797 | 1.00 | 12.08 | A |
| ATOM | 7967 | CD2 | PHE | A | 1016 | 55.823 | 50.366 | −5.751 | 1.00 | 11.67 | A |
| ATOM | 7968 | CE1 | PHE | A | 1016 | 55.561 | 53.115 | −6.046 | 1.00 | 11.87 | A |
| ATOM | 7969 | CE2 | PHE | A | 1016 | 55.549 | 50.912 | −7.008 | 1.00 | 11.94 | A |
| ATOM | 7970 | CZ | PHE | A | 1016 | 55.417 | 52.286 | −7.152 | 1.00 | 11.93 | A |
| ATOM | 7971 | C | PHE | A | 1016 | 55.881 | 48.879 | −1.550 | 1.00 | 13.14 | A |
| ATOM | 7972 | O | PHE | A | 1016 | 57.067 | 48.584 | −1.394 | 1.00 | 13.12 | A |
| ATOM | 7973 | N | LEU | A | 1017 | 54.976 | 48.739 | −0.585 | 1.00 | 13.12 | A |
| ATOM | 7974 | CA | LEU | A | 1017 | 55.369 | 48.299 | 0.751 | 1.00 | 13.58 | A |
| ATOM | 7975 | CB | LEU | A | 1017 | 54.374 | 48.855 | 1.779 | 1.00 | 12.73 | A |
| ATOM | 7976 | CG | LEU | A | 1017 | 54.334 | 50.394 | 1.786 | 1.00 | 12.87 | A |
| ATOM | 7977 | CD1 | LEU | A | 1017 | 53.323 | 50.893 | 2.805 | 1.00 | 12.49 | A |
| ATOM | 7978 | CD2 | LEU | A | 1017 | 55.720 | 50.950 | 2.105 | 1.00 | 12.43 | A |
| ATOM | 7979 | C | LEU | A | 1017 | 55.595 | 46.808 | 0.988 | 1.00 | 14.53 | A |
| ATOM | 7980 | O | LEU | A | 1017 | 56.271 | 46.437 | 1.946 | 1.00 | 15.09 | A |
| ATOM | 7981 | N | GLN | A | 1018 | 55.042 | 45.949 | 0.137 | 1.00 | 15.38 | A |
| ATOM | 7982 | CA | GLN | A | 1018 | 55.246 | 44.513 | 0.310 | 1.00 | 17.15 | A |
| ATOM | 7983 | CB | GLN | A | 1018 | 53.994 | 43.841 | 0.892 | 1.00 | 18.62 | A |
| ATOM | 7984 | CG | GLN | A | 1018 | 54.187 | 42.336 | 1.128 | 1.00 | 21.17 | A |
| ATOM | 7985 | CD | GLN | A | 1018 | 52.903 | 41.592 | 1.466 | 1.00 | 23.14 | A |
| ATOM | 7986 | OE1 | GLN | A | 1018 | 52.917 | 40.373 | 1.668 | 1.00 | 25.29 | A |
| ATOM | 7987 | NE2 | GLN | A | 1018 | 51.789 | 42.313 | 1.522 | 1.00 | 22.60 | A |
| ATOM | 7988 | C | GLN | A | 1018 | 55.608 | 43.822 | −1.003 | 1.00 | 17.88 | A |
| ATOM | 7989 | O | GLN | A | 1018 | 54.975 | 44.054 | −2.033 | 1.00 | 16.65 | A |
| ATOM | 7990 | N | ASN | A | 1019 | 56.628 | 42.971 | −0.963 | 1.00 | 18.66 | A |
| ATOM | 7991 | CA | ASN | A | 1019 | 57.036 | 42.238 | −2.154 | 1.00 | 20.53 | A |
| ATOM | 7992 | CB | ASN | A | 1019 | 58.498 | 41.798 | −2.043 | 1.00 | 21.07 | A |
| ATOM | 7993 | CG | ASN | A | 1019 | 59.446 | 42.971 | −1.879 | 1.00 | 22.32 | A |
| ATOM | 7994 | OD1 | ASN | A | 1019 | 59.313 | 43.991 | −2.561 | 1.00 | 22.36 | A |
| ATOM | 7995 | ND2 | ASN | A | 1019 | 60.419 | 42.830 | −0.982 | 1.00 | 22.05 | A |
| ATOM | 7996 | C | ASN | A | 1019 | 56.127 | 41.022 | −2.285 | 1.00 | 21.52 | A |
| ATOM | 7997 | O | ASN | A | 1019 | 55.948 | 40.267 | −1.330 | 1.00 | 22.36 | A |
| ATOM | 7998 | N | LEU | A | 1020 | 55.548 | 40.838 | −3.466 | 1.00 | 22.06 | A |
| ATOM | 7999 | CA | LEU | A | 1020 | 54.639 | 39.725 | −3.705 | 1.00 | 23.36 | A |
| ATOM | 8000 | CB | LEU | A | 1020 | 53.345 | 40.233 | −4.347 | 1.00 | 23.29 | A |
| ATOM | 8001 | CG | LEU | A | 1020 | 52.546 | 41.316 | −3.618 | 1.00 | 23.35 | A |
| ATOM | 8002 | CD1 | LEU | A | 1020 | 51.427 | 41.815 | −4.524 | 1.00 | 23.65 | A |
| ATOM | 8003 | CD2 | LEU | A | 1020 | 51.983 | 40.763 | −2.320 | 1.00 | 23.16 | A |
| ATOM | 8004 | C | LEU | A | 1020 | 55.238 | 38.648 | −4.600 | 1.00 | 24.39 | A |
| ATOM | 8005 | O | LEU | A | 1020 | 54.876 | 37.474 | −4.491 | 1.00 | 24.00 | A |
| ATOM | 8006 | N | GLU | A | 1021 | 56.145 | 39.044 | −5.488 | 1.00 | 25.74 | A |
| ATOM | 8007 | CA | GLU | A | 1021 | 56.759 | 38.093 | −6.412 | 1.00 | 27.47 | A |
| ATOM | 8008 | CB | GLU | A | 1021 | 55.919 | 37.969 | −7.684 | 1.00 | 28.95 | A |
| ATOM | 8009 | CG | GLU | A | 1021 | 54.604 | 37.243 | −7.551 | 1.00 | 31.78 | A |
| ATOM | 8010 | CD | GLU | A | 1021 | 53.917 | 37.088 | −8.897 | 1.00 | 33.30 | A |
| ATOM | 8011 | OE1 | GLU | A | 1021 | 54.587 | 36.643 | −9.856 | 1.00 | 33.68 | A |
| ATOM | 8012 | OE2 | GLU | A | 1021 | 52.712 | 37.408 | −8.998 | 1.00 | 35.00 | A |
| ATOM | 8013 | C | GLU | A | 1021 | 58.174 | 38.440 | −6.847 | 1.00 | 27.78 | A |
| ATOM | 8014 | O | GLU | A | 1021 | 58.484 | 39.599 | −7.125 | 1.00 | 27.14 | A |
| ATOM | 8015 | N | HIS | A | 1022 | 59.024 | 37.420 | −6.915 | 1.00 | 28.61 | A |
| ATOM | 8016 | CA | HIS | A | 1022 | 60.394 | 37.587 | −7.377 | 1.00 | 29.85 | A |
| ATOM | 8017 | CB | HIS | A | 1022 | 61.343 | 36.652 | −6.624 | 1.00 | 31.37 | A |
| ATOM | 8018 | CG | HIS | A | 1022 | 62.782 | 36.825 | −6.999 | 1.00 | 32.90 | A |
| ATOM | 8019 | CD2 | HIS | A | 1022 | 63.692 | 35.944 | −7.480 | 1.00 | 33.73 | A |
| ATOM | 8020 | ND1 | HIS | A | 1022 | 63.438 | 38.034 | −6.897 | 1.00 | 33.99 | A |
| ATOM | 8021 | CE1 | HIS | A | 1022 | 64.688 | 37.890 | −7.299 | 1.00 | 34.21 | A |
| ATOM | 8022 | NE2 | HIS | A | 1022 | 64.868 | 36.631 | −7.659 | 1.00 | 34.14 | A |
| ATOM | 8023 | C | HIS | A | 1022 | 60.298 | 37.186 | −8.847 | 1.00 | 29.97 | A |
| ATOM | 8024 | O | HIS | A | 1022 | 60.054 | 36.022 | −9.169 | 1.00 | 29.65 | A |
| ATOM | 8025 | N | LEU | A | 1023 | 60.477 | 38.159 | −9.732 | 1.00 | 29.90 | A |
| ATOM | 8026 | CA | LEU | A | 1023 | 60.354 | 37.929 | −11.165 | 1.00 | 30.66 | A |
| ATOM | 8027 | CB | LEU | A | 1023 | 59.901 | 39.225 | −11.837 | 1.00 | 30.06 | A |
| ATOM | 8028 | CG | LEU | A | 1023 | 58.644 | 39.818 | −11.194 | 1.00 | 29.45 | A |
| ATOM | 8029 | CD1 | LEU | A | 1023 | 58.367 | 41.198 | −11.757 | 1.00 | 30.04 | A |
| ATOM | 8030 | CD2 | LEU | A | 1023 | 57.466 | 38.886 | −11.432 | 1.00 | 29.81 | A |
| ATOM | 8031 | C | LEU | A | 1023 | 61.603 | 37.398 | −11.854 | 1.00 | 31.31 | A |
| ATOM | 8032 | O | LEU | A | 1023 | 62.656 | 38.034 | −11.836 | 1.00 | 30.70 | A |
| ATOM | 8033 | N | ASP | A | 1024 | 61.470 | 36.229 | −12.470 | 1.00 | 32.74 | A |
| ATOM | 8034 | CA | ASP | A | 1024 | 62.583 | 35.610 | −13.179 | 1.00 | 33.94 | A |
| ATOM | 8035 | CB | ASP | A | 1024 | 62.202 | 34.203 | −13.646 | 1.00 | 36.11 | A |
| ATOM | 8036 | CG | ASP | A | 1024 | 62.346 | 33.163 | −12.547 | 1.00 | 38.17 | A |
| ATOM | 8037 | OD1 | ASP | A | 1024 | 62.074 | 31.974 | −12.818 | 1.00 | 40.17 | A |
| ATOM | 8038 | OD2 | ASP | A | 1024 | 62.737 | 33.531 | −11.417 | 1.00 | 39.44 | A |
| ATOM | 8039 | C | ASP | A | 1024 | 63.001 | 36.454 | −14.373 | 1.00 | 33.60 | A |
| ATOM | 8040 | O | ASP | A | 1024 | 62.179 | 37.150 | −14.973 | 1.00 | 33.92 | A |
| ATOM | 8041 | N | GLY | A | 1025 | 64.284 | 36.393 | −14.712 | 1.00 | 33.05 | A |
| ATOM | 8042 | CA | GLY | A | 1025 | 64.788 | 37.165 | −15.832 | 1.00 | 32.63 | A |
| ATOM | 8043 | C | GLY | A | 1025 | 64.714 | 38.650 | −15.547 | 1.00 | 32.42 | A |
| ATOM | 8044 | O | GLY | A | 1025 | 65.062 | 39.474 | −16.394 | 1.00 | 32.65 | A |
| ATOM | 8045 | N | MET | A | 1026 | 64.262 | 38.990 | −14.344 | 1.00 | 31.91 | A |
| ATOM | 8046 | CA | MET | A | 1026 | 64.135 | 40.381 | −13.932 | 1.00 | 31.78 | A |
| ATOM | 8047 | CB | MET | A | 1026 | 65.515 | 41.036 | −13.860 | 1.00 | 32.40 | A |
| ATOM | 8048 | CG | MET | A | 1026 | 66.511 | 40.278 | −13.002 | 1.00 | 33.57 | A |
| ATOM | 8049 | SD | MET | A | 1026 | 68.098 | 41.116 | −12.885 | 1.00 | 35.28 | A |
| ATOM | 8050 | CE | MET | A | 1026 | 68.895 | 40.532 | −14.379 | 1.00 | 34.34 | A |
| ATOM | 8051 | C | MET | A | 1026 | 63.254 | 41.135 | −14.920 | 1.00 | 31.27 | A |
| ATOM | 8052 | O | MET | A | 1026 | 63.496 | 42.302 | −15.221 | 1.00 | 30.28 | A |
| ATOM | 8053 | N | VAL | A | 1027 | 62.228 | 40.457 | −15.422 | 1.00 | 30.91 | A |
| ATOM | 8054 | CA | VAL | A | 1027 | 61.316 | 41.068 | −16.377 | 1.00 | 31.71 | A |
| ATOM | 8055 | CB | VAL | A | 1027 | 61.178 | 40.212 | −17.650 | 1.00 | 31.61 | A |
| ATOM | 8056 | CG1 | VAL | A | 1027 | 60.155 | 40.839 | −18.589 | 1.00 | 32.07 | A |
| ATOM | 8057 | CG2 | VAL | A | 1027 | 62.524 | 40.089 | −18.340 | 1.00 | 31.52 | A |
| ATOM | 8058 | C | VAL | A | 1027 | 59.929 | 41.273 | −15.788 | 1.00 | 31.69 | A |
| ATOM | 8059 | O | VAL | A | 1027 | 59.295 | 40.327 | −15.320 | 1.00 | 31.57 | A |
| ATOM | 8060 | N | ALA | A | 1028 | 59.463 | 42.516 | −15.814 | 1.00 | 31.73 | A |
| ATOM | 8061 | CA | ALA | A | 1028 | 58.140 | 42.839 | −15.300 | 1.00 | 32.08 | A |
| ATOM | 8062 | CB | ALA | A | 1028 | 58.114 | 44.265 | −14.754 | 1.00 | 32.15 | A |
| ATOM | 8063 | C | ALA | A | 1028 | 57.145 | 42.694 | −16.441 | 1.00 | 31.91 | A |
| ATOM | 8064 | O | ALA | A | 1028 | 57.116 | 43.514 | −17.361 | 1.00 | 32.68 | A |
| ATOM | 8065 | N | PRO | A | 1029 | 56.323 | 41.638 | −16.405 | 1.00 | 31.39 | A |
| ATOM | 8066 | CD | PRO | A | 1029 | 56.223 | 40.615 | −15.348 | 1.00 | 31.43 | A |
| ATOM | 8067 | CA | PRO | A | 1029 | 55.327 | 41.406 | −17.454 | 1.00 | 31.08 | A |
| ATOM | 8068 | CB | PRO | A | 1029 | 54.819 | 40.005 | −17.132 | 1.00 | 31.27 | A |
| ATOM | 8069 | CG | PRO | A | 1029 | 54.878 | 39.979 | −15.639 | 1.00 | 31.48 | A |
| ATOM | 8070 | C | PRO | A | 1029 | 54.217 | 42.454 | −17.398 | 1.00 | 30.48 | A |
| ATOM | 8071 | O | PRO | A | 1029 | 53.919 | 42.995 | −16.334 | 1.00 | 30.65 | A |
| ATOM | 8072 | N | GLU | A | 1030 | 53.612 | 42.743 | −18.545 | 1.00 | 29.57 | A |
| ATOM | 8073 | CA | GLU | A | 1030 | 52.539 | 43.725 | −18.595 | 1.00 | 28.77 | A |
| ATOM | 8074 | CB | GLU | A | 1030 | 52.224 | 44.094 | −20.047 | 1.00 | 29.04 | A |
| ATOM | 8075 | CG | GLU | A | 1030 | 53.447 | 44.504 | −20.861 | 1.00 | 28.71 | A |
| ATOM | 8076 | CD | GLU | A | 1030 | 53.086 | 44.989 | −22.253 | 1.00 | 28.71 | A |
| ATOM | 8077 | OE1 | GLU | A | 1030 | 52.176 | 44.395 | −22.867 | 1.00 | 29.16 | A |
| ATOM | 8078 | OE2 | GLU | A | 1030 | 53.716 | 45.952 | −22.738 | 1.00 | 27.83 | A |
| ATOM | 8079 | C | GLU | A | 1030 | 51.300 | 43.153 | −17.914 | 1.00 | 27.90 | A |
| ATOM | 8080 | O | GLU | A | 1030 | 51.190 | 41.939 | −17.717 | 1.00 | 28.78 | A |
| ATOM | 8081 | N | VAL | A | 1031 | 50.366 | 44.026 | −17.558 | 1.00 | 26.52 | A |
| ATOM | 8082 | CA | VAL | A | 1031 | 49.153 | 43.595 | −16.883 | 1.00 | 24.32 | A |
| ATOM | 8083 | CB | VAL | A | 1031 | 48.745 | 44.606 | −15.791 | 1.00 | 25.20 | A |
| ATOM | 8084 | CG1 | VAL | A | 1031 | 49.850 | 44.709 | −14.752 | 1.00 | 25.47 | A |
| ATOM | 8085 | CG2 | VAL | A | 1031 | 48.466 | 45.961 | −16.408 | 1.00 | 24.31 | A |
| ATOM | 8086 | C | VAL | A | 1031 | 47.977 | 43.388 | −17.826 | 1.00 | 22.92 | A |
| ATOM | 8087 | O | VAL | A | 1031 | 48.032 | 43.752 | −18.998 | 1.00 | 22.93 | A |
| ATOM | 8088 | N | CYS | A | 1032 | 46.917 | 42.790 | −17.295 | 1.00 | 21.33 | A |
| ATOM | 8089 | CA | CYS | A | 1032 | 45.703 | 42.511 | −18.051 | 1.00 | 20.05 | A |
| ATOM | 8090 | C | CYS | A | 1032 | 44.804 | 43.740 | −18.132 | 1.00 | 18.10 | A |
| ATOM | 8091 | O | CYS | A | 1032 | 44.982 | 44.698 | −17.381 | 1.00 | 17.38 | A |
| ATOM | 8092 | CB | CYS | A | 1032 | 44.923 | 41.377 | −17.380 | 1.00 | 20.75 | A |
| ATOM | 8093 | SG | CYS | A | 1032 | 45.684 | 39.722 | −17.453 | 1.00 | 22.84 | A |
| ATOM | 8094 | N | PRO | A | 1033 | 43.820 | 43.727 | −19.049 | 1.00 | 16.57 | A |
| ATOM | 8095 | CD | PRO | A | 1033 | 43.568 | 42.726 | −20.104 | 1.00 | 16.27 | A |
| ATOM | 8096 | CA | PRO | A | 1033 | 42.906 | 44.865 | −19.183 | 1.00 | 16.00 | A |
| ATOM | 8097 | CB | PRO | A | 1033 | 41.941 | 44.404 | −20.275 | 1.00 | 15.70 | A |
| ATOM | 8098 | CG | PRO | A | 1033 | 42.811 | 43.529 | −21.138 | 1.00 | 16.00 | A |
| ATOM | 8099 | C | PRO | A | 1033 | 42.192 | 45.116 | −17.853 | 1.00 | 15.35 | A |
| ATOM | 8100 | O | PRO | A | 1033 | 41.725 | 44.178 | −17.206 | 1.00 | 14.40 | A |
| ATOM | 8101 | N | MET | A | 1034 | 42.119 | 46.385 | −17.462 | 1.00 | 15.28 | A |
| ATOM | 8102 | CA | MET | A | 1034 | 41.482 | 46.815 | −16.221 | 1.00 | 15.94 | A |
| ATOM | 8103 | CB | MET | A | 1034 | 40.041 | 46.308 | −16.143 | 1.00 | 15.40 | A |
| ATOM | 8104 | CG | MET | A | 1034 | 39.132 | 46.875 | −17.223 | 1.00 | 16.32 | A |
| ATOM | 8105 | SD | MET | A | 1034 | 39.209 | 48.680 | −17.336 | 1.00 | 16.96 | A |
| ATOM | 8106 | CE | MET | A | 1034 | 38.245 | 49.170 | −15.883 | 1.00 | 17.76 | A |
| ATOM | 8107 | C | MET | A | 1034 | 42.246 | 46.391 | −14.972 | 1.00 | 16.88 | A |
| ATOM | 8108 | O | MET | A | 1034 | 41.726 | 46.484 | −13.860 | 1.00 | 17.93 | A |
| ATOM | 8109 | N | GLU | A | 1035 | 43.477 | 45.930 | −15.161 | 1.00 | 16.50 | A |
| ATOM | 8110 | CA | GLU | A | 1035 | 44.318 | 45.517 | −14.044 | 1.00 | 17.13 | A |
| ATOM | 8111 | CB | GLU | A | 1035 | 44.989 | 44.166 | −14.342 | 1.00 | 19.35 | A |
| ATOM | 8112 | CG | GLU | A | 1035 | 46.198 | 43.864 | −13.453 | 1.00 | 23.20 | A |
| ATOM | 8113 | CD | GLU | A | 1035 | 46.687 | 42.421 | −13.550 | 1.00 | 25.99 | A |
| ATOM | 8114 | OE1 | GLU | A | 1035 | 46.973 | 41.943 | −14.674 | 1.00 | 26.11 | A |
| ATOM | 8115 | OE2 | GLU | A | 1035 | 46.794 | 41.766 | −12.488 | 1.00 | 28.07 | A |
| ATOM | 8116 | C | GLU | A | 1035 | 45.386 | 46.570 | −13.765 | 1.00 | 16.00 | A |
| ATOM | 8117 | O | GLU | A | 1035 | 45.777 | 47.328 | −14.652 | 1.00 | 14.36 | A |
| ATOM | 8118 | N | THR | A | 1036 | 45.839 | 46.620 | −12.518 | 1.00 | 15.22 | A |
| ATOM | 8119 | CA | THR | A | 1036 | 46.881 | 47.554 | −12.116 | 1.00 | 14.15 | A |
| ATOM | 8120 | CB | THR | A | 1036 | 46.323 | 48.705 | −11.253 | 1.00 | 13.96 | A |
| ATOM | 8121 | OG1 | THR | A | 1036 | 45.285 | 49.383 | −11.967 | 1.00 | 13.25 | A |
| ATOM | 8122 | CG2 | THR | A | 1036 | 47.427 | 49.701 | −10.914 | 1.00 | 13.39 | A |
| ATOM | 8123 | C | THR | A | 1036 | 47.877 | 46.768 | −11.279 | 1.00 | 13.98 | A |
| ATOM | 8124 | O | THR | A | 1036 | 47.487 | 46.031 | −10.375 | 1.00 | 13.86 | A |
| ATOM | 8125 | N | ALA | A | 1037 | 49.159 | 46.912 | −11.584 | 1.00 | 13.57 | A |
| ATOM | 8126 | CA | ALA | A | 1037 | 50.186 | 46.213 | −10.832 | 1.00 | 14.07 | A |
| ATOM | 8127 | CB | ALA | A | 1037 | 50.706 | 45.022 | −11.626 | 1.00 | 14.55 | A |
| ATOM | 8128 | C | ALA | A | 1037 | 51.320 | 47.171 | −10.526 | 1.00 | 14.09 | A |
| ATOM | 8129 | O | ALA | A | 1037 | 51.442 | 48.226 | −11.149 | 1.00 | 14.87 | A |
| ATOM | 8130 | N | ALA | A | 1038 | 52.134 | 46.810 | −9.544 | 1.00 | 13.48 | A |
| ATOM | 8131 | CA | ALA | A | 1038 | 53.268 | 47.633 | −9.164 | 1.00 | 13.29 | A |
| ATOM | 8132 | CB | ALA | A | 1038 | 53.066 | 48.207 | −7.774 | 1.00 | 12.35 | A |
| ATOM | 8133 | C | ALA | A | 1038 | 54.512 | 46.766 | −9.193 | 1.00 | 13.30 | A |
| ATOM | 8134 | O | ALA | A | 1038 | 54.468 | 45.590 | −8.823 | 1.00 | 13.16 | A |
| ATOM | 8135 | N | TYR | A | 1039 | 55.612 | 47.347 | −9.655 | 1.00 | 13.78 | A |
| ATOM | 8136 | CA | TYR | A | 1039 | 56.879 | 46.642 | −9.722 | 1.00 | 14.56 | A |
| ATOM | 8137 | CB | TYR | A | 1039 | 57.218 | 46.242 | −11.160 | 1.00 | 14.67 | A |
| ATOM | 8138 | CG | TYR | A | 1039 | 56.188 | 45.367 | −11.836 | 1.00 | 16.22 | A |
| ATOM | 8139 | CD1 | TYR | A | 1039 | 55.247 | 45.914 | −12.707 | 1.00 | 17.34 | A |
| ATOM | 8140 | CE1 | TYR | A | 1039 | 54.306 | 45.111 | −13.349 | 1.00 | 18.80 | A |
| ATOM | 8141 | CD2 | TYR | A | 1039 | 56.162 | 43.990 | −11.616 | 1.00 | 17.38 | A |
| ATOM | 8142 | CE2 | TYR | A | 1039 | 55.223 | 43.178 | −12.253 | 1.00 | 18.40 | A |
| ATOM | 8143 | CZ | TYR | A | 1039 | 54.301 | 43.745 | −13.118 | 1.00 | 19.04 | A |
| ATOM | 8144 | OH | TYR | A | 1039 | 53.377 | 42.949 | −13.758 | 1.00 | 21.01 | A |
| ATOM | 8145 | C | TYR | A | 1039 | 57.979 | 47.546 | −9.204 | 1.00 | 14.90 | A |
| ATOM | 8146 | O | TYR | A | 1039 | 57.925 | 48.767 | −9.362 | 1.00 | 14.95 | A |
| ATOM | 8147 | N | VAL | A | 1040 | 58.977 | 46.936 | −8.580 | 1.00 | 15.13 | A |
| ATOM | 8148 | CA | VAL | A | 1040 | 60.115 | 47.674 | −8.064 | 1.00 | 15.16 | A |
| ATOM | 8149 | CB | VAL | A | 1040 | 60.176 | 47.631 | −6.521 | 1.00 | 15.50 | A |
| ATOM | 8150 | CG1 | VAL | A | 1040 | 61.472 | 48.281 | −6.029 | 1.00 | 15.65 | A |
| ATOM | 8151 | CG2 | VAL | A | 1040 | 58.975 | 48.363 | −5.937 | 1.00 | 14.36 | A |
| ATOM | 8152 | C | VAL | A | 1040 | 61.357 | 47.010 | −8.635 | 1.00 | 16.26 | A |
| ATOM | 8153 | O | VAL | A | 1040 | 61.538 | 45.795 | −8.510 | 1.00 | 16.39 | A |
| ATOM | 8154 | N | SER | A | 1041 | 62.197 | 47.802 | −9.291 | 1.00 | 16.31 | A |
| ATOM | 8155 | CA | SER | A | 1041 | 63.430 | 47.283 | −9.860 | 1.00 | 17.09 | A |
| ATOM | 8156 | CB | SER | A | 1041 | 63.615 | 47.784 | −11.299 | 1.00 | 16.94 | A |
| ATOM | 8157 | OG | SER | A | 1041 | 63.690 | 49.198 | −11.352 | 1.00 | 16.34 | A |
| ATOM | 8158 | C | SER | A | 1041 | 64.585 | 47.755 | −8.987 | 1.00 | 17.15 | A |
| ATOM | 8159 | O | SER | A | 1041 | 64.618 | 48.910 | −8.566 | 1.00 | 16.29 | A |
| ATOM | 8160 | N | SER | A | 1042 | 65.520 | 46.851 | −8.706 | 1.00 | 17.84 | A |
| ATOM | 8161 | CA | SER | A | 1042 | 66.690 | 47.171 | −7.890 | 1.00 | 18.46 | A |
| ATOM | 8162 | CB | SER | A | 1042 | 66.896 | 46.108 | −6.810 | 1.00 | 18.36 | A |
| ATOM | 8163 | OG | SER | A | 1042 | 65.761 | 46.045 | −5.961 | 1.00 | 19.21 | A |
| ATOM | 8164 | C | SER | A | 1042 | 67.905 | 47.240 | −8.802 | 1.00 | 18.79 | A |
| ATOM | 8165 | O | SER | A | 1042 | 68.056 | 46.419 | −9.707 | 1.00 | 18.69 | A |
| ATOM | 8166 | N | HIS | A | 1043 | 68.766 | 48.223 | −8.563 | 1.00 | 19.78 | A |
| ATOM | 8167 | CA | HIS | A | 1043 | 69.946 | 48.420 | −9.397 | 1.00 | 20.82 | A |
| ATOM | 8168 | CB | HIS | A | 1043 | 69.749 | 49.682 | −10.234 | 1.00 | 19.47 | A |
| ATOM | 8169 | CG | HIS | A | 1043 | 68.444 | 49.704 | −10.966 | 1.00 | 18.50 | A |
| ATOM | 8170 | CD2 | HIS | A | 1043 | 67.209 | 50.092 | −10.569 | 1.00 | 17.78 | A |
| ATOM | 8171 | ND1 | HIS | A | 1043 | 68.292 | 49.189 | −12.236 | 1.00 | 17.74 | A |
| ATOM | 8172 | CE1 | HIS | A | 1043 | 67.021 | 49.254 | −12.587 | 1.00 | 17.63 | A |
| ATOM | 8173 | NE2 | HIS | A | 1043 | 66.342 | 49.797 | −11.593 | 1.00 | 18.16 | A |
| ATOM | 8174 | C | HIS | A | 1043 | 71.215 | 48.521 | −8.565 | 1.00 | 22.62 | A |
| ATOM | 8175 | O | HIS | A | 1043 | 71.266 | 49.246 | −7.573 | 1.00 | 22.60 | A |
| ATOM | 8176 | N | SER | A | 1044 | 72.240 | 47.790 | −8.987 | 1.00 | 24.94 | A |
| ATOM | 8177 | CA | SER | A | 1044 | 73.509 | 47.767 | −8.275 | 1.00 | 27.40 | A |
| ATOM | 8178 | CB | SER | A | 1044 | 74.217 | 46.432 | −8.532 | 1.00 | 28.13 | A |
| ATOM | 8179 | OG | SER | A | 1044 | 74.193 | 46.096 | −9.911 | 1.00 | 29.65 | A |
| ATOM | 8180 | C | SER | A | 1044 | 74.434 | 48.923 | −8.632 | 1.00 | 28.23 | A |
| ATOM | 8181 | O | SER | A | 1044 | 74.039 | 49.786 | −9.448 | 1.00 | 28.56 | A |
| ATOM | 8182 | OXT | SER | A | 1044 | 75.552 | 48.944 | −8.077 | 1.00 | 29.86 | A |
| ATOM | 8183 | OH2 | WAT | W | 1 | 41.979 | 63.654 | −7.156 | 1.00 | 8.05 | W |
| ATOM | 8184 | OH2 | WAT | W | 2 | 53.601 | 65.006 | −19.784 | 1.00 | 11.44 | W |
| ATOM | 8185 | OH2 | WAT | W | 3 | 39.162 | 63.039 | −19.188 | 1.00 | 9.94 | W |
| ATOM | 8186 | OH2 | WAT | W | 4 | 52.119 | 54.293 | −4.860 | 1.00 | 9.88 | W |
| ATOM | 8187 | OH2 | WAT | W | 5 | 56.136 | 53.559 | −0.657 | 1.00 | 10.79 | W |
| ATOM | 8188 | OH2 | WAT | W | 6 | 31.383 | 50.075 | −24.059 | 1.00 | 10.96 | W |
| ATOM | 8189 | OH2 | WAT | W | 7 | 49.858 | 48.630 | 1.284 | 1.00 | 12.86 | W |
| ATOM | 8190 | OH2 | WAT | W | 8 | 36.992 | 57.884 | 13.375 | 1.00 | 9.63 | W |
| ATOM | 8191 | OH2 | WAT | W | 9 | 26.755 | 69.172 | −9.245 | 1.00 | 11.38 | W |
| ATOM | 8192 | OH2 | WAT | W | 10 | 39.324 | 64.859 | −14.901 | 1.00 | 12.67 | W |
| ATOM | 8193 | OH2 | WAT | W | 11 | 34.198 | 58.783 | −8.711 | 1.00 | 11.93 | W |
| ATOM | 8194 | OH2 | WAT | W | 12 | 60.950 | 59.827 | −8.309 | 1.00 | 11.38 | W |
| ATOM | 8195 | OH2 | WAT | W | 13 | 36.628 | 72.825 | 0.199 | 1.00 | 11.49 | W |
| ATOM | 8196 | OH2 | WAT | W | 14 | 31.708 | 47.175 | −8.092 | 1.00 | 10.89 | W |
| ATOM | 8197 | OH2 | WAT | W | 15 | 30.142 | 55.929 | 17.238 | 1.00 | 10.75 | W |
| ATOM | 8198 | OH2 | WAT | W | 16 | 26.117 | 49.830 | −13.740 | 1.00 | 10.75 | W |
| ATOM | 8199 | OH2 | WAT | W | 17 | 37.632 | 52.396 | −21.150 | 1.00 | 12.30 | W |
| ATOM | 8200 | OH2 | WAT | W | 18 | 33.010 | 62.954 | 0.153 | 1.00 | 10.82 | W |
| ATOM | 8201 | OH2 | WAT | W | 19 | 24.689 | 53.037 | −11.753 | 1.00 | 11.02 | W |
| ATOM | 8202 | OH2 | WAT | W | 20 | 63.105 | 61.515 | −7.581 | 1.00 | 10.48 | W |
| ATOM | 8203 | OH2 | WAT | W | 21 | 41.285 | 59.153 | 13.532 | 1.00 | 12.85 | W |
| ATOM | 8204 | OH2 | WAT | W | 22 | 47.287 | 55.959 | −15.370 | 1.00 | 14.81 | W |
| ATOM | 8205 | OH2 | WAT | W | 23 | 56.380 | 55.892 | −2.106 | 1.00 | 8.54 | W |
| ATOM | 8206 | OH2 | WAT | W | 24 | 67.351 | 60.850 | −5.664 | 1.00 | 14.58 | W |
| ATOM | 8207 | OH2 | WAT | W | 25 | 26.262 | 48.939 | −10.894 | 1.00 | 13.65 | W |
| ATOM | 8208 | OH2 | WAT | W | 26 | 65.620 | 60.396 | −7.599 | 1.00 | 14.15 | W |
| ATOM | 8209 | OH2 | WAT | W | 27 | 32.469 | 60.318 | −1.870 | 1.00 | 10.93 | W |
| ATOM | 8210 | OH2 | WAT | W | 28 | 20.127 | 54.851 | 16.211 | 1.00 | 17.01 | W |
| ATOM | 8211 | OH2 | WAT | W | 29 | 23.824 | 55.676 | −23.371 | 1.00 | 12.86 | W |
| ATOM | 8212 | OH2 | WAT | W | 30 | 39.335 | 57.550 | 14.798 | 1.00 | 11.81 | W |
| ATOM | 8213 | OH2 | WAT | W | 31 | 20.371 | 58.963 | −21.881 | 1.00 | 12.24 | W |
| ATOM | 8214 | OH2 | WAT | W | 32 | 28.075 | 61.166 | 19.435 | 1.00 | 13.45 | W |
| ATOM | 8215 | OH2 | WAT | W | 33 | 34.058 | 56.487 | −26.256 | 1.00 | 11.49 | W |
| ATOM | 8216 | OH2 | WAT | W | 34 | 26.341 | 40.112 | 7.993 | 1.00 | 15.36 | W |
| ATOM | 8217 | OH2 | WAT | W | 35 | 63.796 | 50.324 | −14.022 | 1.00 | 12.67 | W |
| ATOM | 8218 | OH2 | WAT | W | 36 | 37.486 | 57.200 | 1.752 | 1.00 | 12.39 | W |
| ATOM | 8219 | OH2 | WAT | W | 37 | 24.088 | 42.135 | 6.847 | 1.00 | 14.18 | W |
| ATOM | 8220 | OH2 | WAT | W | 38 | 31.966 | 65.565 | 18.975 | 1.00 | 15.73 | W |
| ATOM | 8221 | OH2 | WAT | W | 39 | 51.492 | 56.797 | −5.607 | 1.00 | 8.86 | W |
| ATOM | 8222 | OH2 | WAT | W | 40 | 20.056 | 56.197 | 7.115 | 1.00 | 13.43 | W |
| ATOM | 8223 | OH2 | WAT | W | 41 | 28.261 | 43.988 | 13.133 | 1.00 | 11.93 | W |
| ATOM | 8224 | OH2 | WAT | W | 42 | 30.237 | 58.632 | −11.794 | 1.00 | 11.17 | W |
| ATOM | 8225 | OH2 | WAT | W | 43 | 64.887 | 59.396 | −3.618 | 1.00 | 16.12 | W |
| ATOM | 8226 | OH2 | WAT | W | 44 | 46.340 | 67.858 | −15.581 | 1.00 | 15.57 | W |
| ATOM | 8227 | OH2 | WAT | W | 45 | 60.698 | 58.457 | −2.874 | 1.00 | 13.59 | W |
| ATOM | 8228 | OH2 | WAT | W | 46 | 60.499 | 62.267 | −1.276 | 1.00 | 14.53 | W |
| ATOM | 8229 | OH2 | WAT | W | 47 | 53.593 | 60.068 | −9.458 | 1.00 | 12.82 | W |
| ATOM | 8230 | OH2 | WAT | W | 48 | 18.566 | 51.203 | −12.450 | 1.00 | 15.00 | W |
| ATOM | 8231 | OH2 | WAT | W | 49 | 28.876 | 42.922 | −11.951 | 1.00 | 16.11 | W |
| ATOM | 8232 | OH2 | WAT | W | 50 | 34.497 | 79.288 | −9.194 | 1.00 | 12.70 | W |
| ATOM | 8233 | OH2 | WAT | W | 51 | 21.521 | 58.048 | −7.816 | 1.00 | 13.55 | W |
| ATOM | 8234 | OH2 | WAT | W | 52 | 36.974 | 72.026 | −4.632 | 1.00 | 11.46 | W |
| ATOM | 8235 | OH2 | WAT | W | 53 | 55.633 | 59.600 | −11.527 | 1.00 | 12.06 | W |
| ATOM | 8236 | OH2 | WAT | W | 54 | 36.685 | 44.992 | −5.521 | 1.00 | 12.94 | W |
| ATOM | 8237 | OH2 | WAT | W | 55 | 51.282 | 53.464 | −24.055 | 1.00 | 15.04 | W |
| ATOM | 8238 | OH2 | WAT | W | 56 | 35.178 | 50.782 | −17.540 | 1.00 | 18.13 | W |
| ATOM | 8239 | OH2 | WAT | W | 57 | 60.506 | 54.958 | −0.396 | 1.00 | 15.58 | W |
| ATOM | 8240 | OH2 | WAT | W | 58 | 42.909 | 56.986 | 6.187 | 1.00 | 11.90 | W |
| ATOM | 8241 | OH2 | WAT | W | 59 | 47.858 | 59.457 | −25.679 | 1.00 | 15.94 | W |
| ATOM | 8242 | OH2 | WAT | W | 60 | 37.848 | 73.645 | −2.364 | 1.00 | 13.96 | W |
| ATOM | 8243 | OH2 | WAT | W | 61 | 19.391 | 55.464 | 11.518 | 1.00 | 14.93 | W |
| ATOM | 8244 | OH2 | WAT | W | 62 | 25.911 | 39.813 | 5.195 | 1.00 | 11.56 | W |
| ATOM | 8245 | OH2 | WAT | W | 63 | 23.258 | 54.236 | −3.647 | 1.00 | 15.92 | W |
| ATOM | 8246 | OH2 | WAT | W | 64 | 34.294 | 52.614 | −1.083 | 1.00 | 12.31 | W |
| ATOM | 8247 | OH2 | WAT | W | 65 | 50.997 | 44.377 | −7.959 | 1.00 | 13.40 | W |
| ATOM | 8248 | OH2 | WAT | W | 66 | 37.875 | 59.168 | 11.062 | 1.00 | 12.70 | W |
| ATOM | 8249 | OH2 | WAT | W | 67 | 16.619 | 49.176 | 24.039 | 1.00 | 17.09 | W |
| ATOM | 8250 | OH2 | WAT | W | 68 | 19.392 | 53.623 | 8.170 | 1.00 | 14.32 | W |
| ATOM | 8251 | OH2 | WAT | W | 69 | 11.769 | 51.733 | 14.377 | 1.00 | 15.40 | W |
| ATOM | 8252 | OH2 | WAT | W | 70 | 17.139 | 56.383 | 10.249 | 1.00 | 14.93 | W |
| ATOM | 8253 | OH2 | WAT | W | 71 | 67.983 | 65.707 | −3.476 | 1.00 | 14.66 | W |
| ATOM | 8254 | OH2 | WAT | W | 72 | 22.822 | 53.249 | −24.440 | 1.00 | 18.17 | W |
| ATOM | 8255 | OH2 | WAT | W | 73 | 52.061 | 50.772 | 6.360 | 1.00 | 11.54 | W |
| ATOM | 8256 | OH2 | WAT | W | 74 | 17.596 | 52.950 | 6.333 | 1.00 | 13.94 | W |
| ATOM | 8257 | OH2 | WAT | W | 75 | 68.801 | 58.401 | −5.067 | 1.00 | 15.47 | W |
| ATOM | 8258 | OH2 | WAT | W | 76 | 33.510 | 48.227 | −10.155 | 1.00 | 10.97 | W |
| ATOM | 8259 | OH2 | WAT | W | 77 | 26.793 | 41.647 | −13.173 | 1.00 | 13.87 | W |
| ATOM | 8260 | OH2 | WAT | W | 78 | 26.744 | 61.077 | −3.285 | 1.00 | 11.60 | W |
| ATOM | 8261 | OH2 | WAT | W | 79 | 49.480 | 62.451 | −8.644 | 1.00 | 9.33 | W |
| ATOM | 8262 | OH2 | WAT | W | 80 | 41.021 | 52.499 | −16.991 | 1.00 | 13.50 | W |
| ATOM | 8263 | OH2 | WAT | W | 81 | 31.434 | 76.936 | −6.264 | 1.00 | 15.03 | W |
| ATOM | 8264 | OH2 | WAT | W | 82 | 43.379 | 68.423 | −24.915 | 1.00 | 14.16 | W |
| ATOM | 8265 | OH2 | WAT | W | 83 | 32.764 | 81.302 | −11.715 | 1.00 | 13.97 | W |
| ATOM | 8266 | OH2 | WAT | W | 84 | 20.523 | 66.419 | −19.639 | 1.00 | 13.09 | W |
| ATOM | 8267 | OH2 | WAT | W | 85 | 40.401 | 62.934 | 6.179 | 1.00 | 12.03 | W |
| ATOM | 8268 | OH2 | WAT | W | 86 | 42.119 | 47.453 | −24.559 | 1.00 | 17.53 | W |
| ATOM | 8269 | OH2 | WAT | W | 87 | 44.524 | 42.002 | −0.591 | 1.00 | 19.33 | W |
| ATOM | 8270 | OH2 | WAT | W | 88 | 38.626 | 55.309 | −31.696 | 1.00 | 13.53 | W |
| ATOM | 8271 | OH2 | WAT | W | 89 | 22.307 | 54.475 | −11.945 | 1.00 | 12.50 | W |
| ATOM | 8272 | OH2 | WAT | W | 90 | 48.303 | 89.462 | −30.628 | 1.00 | 20.34 | W |
| ATOM | 8273 | OH2 | WAT | W | 91 | 36.707 | 51.821 | 3.758 | 1.00 | 11.30 | W |
| ATOM | 8274 | OH2 | WAT | W | 92 | 38.733 | 62.443 | 8.416 | 1.00 | 13.40 | W |
| ATOM | 8275 | OH2 | WAT | W | 93 | 47.443 | 46.140 | 3.623 | 1.00 | 9.98 | W |
| ATOM | 8276 | OH2 | WAT | W | 94 | 33.576 | 57.296 | −35.875 | 1.00 | 16.19 | W |
| ATOM | 8277 | OH2 | WAT | W | 95 | 32.312 | 77.752 | −29.560 | 1.00 | 14.95 | W |
| ATOM | 8278 | OH2 | WAT | W | 96 | 39.250 | 39.226 | 17.190 | 1.00 | 18.72 | W |
| ATOM | 8279 | OH2 | WAT | W | 97 | 35.930 | 53.330 | 1.320 | 1.00 | 9.48 | W |
| ATOM | 8280 | OH2 | WAT | W | 98 | 14.447 | 60.154 | 5.058 | 1.00 | 17.17 | W |
| ATOM | 8281 | OH2 | WAT | W | 99 | 21.655 | 57.292 | 5.020 | 1.00 | 11.98 | W |
| ATOM | 8282 | OH2 | WAT | W | 100 | 43.040 | 48.234 | 11.162 | 1.00 | 12.84 | W |
| ATOM | 8283 | OH2 | WAT | W | 101 | 26.080 | 73.018 | 5.559 | 1.00 | 14.30 | W |
| ATOM | 8284 | OH2 | WAT | W | 102 | 27.431 | 48.649 | −21.557 | 1.00 | 22.43 | W |
| ATOM | 8285 | OH2 | WAT | W | 103 | 33.294 | 78.869 | −4.985 | 1.00 | 13.70 | W |
| ATOM | 8286 | OH2 | WAT | W | 104 | 49.992 | 59.656 | −11.582 | 1.00 | 9.75 | W |
| ATOM | 8287 | OH2 | WAT | W | 105 | 25.582 | 51.390 | −9.530 | 1.00 | 14.63 | W |
| ATOM | 8288 | OH2 | WAT | W | 106 | 40.201 | 56.515 | 5.849 | 1.00 | 11.03 | W |
| ATOM | 8289 | OH2 | WAT | W | 107 | 14.015 | 59.894 | −3.431 | 1.00 | 15.09 | W |
| ATOM | 8290 | OH2 | WAT | W | 108 | 19.701 | 53.654 | −11.750 | 1.00 | 14.70 | W |
| ATOM | 8291 | OH2 | WAT | W | 109 | 26.513 | 63.805 | 17.151 | 1.00 | 21.71 | W |
| ATOM | 8292 | OH2 | WAT | W | 110 | 19.778 | 57.568 | −11.786 | 1.00 | 15.33 | W |
| ATOM | 8293 | OH2 | WAT | W | 111 | 47.187 | 48.533 | 11.420 | 1.00 | 22.52 | W |
| ATOM | 8294 | OH2 | WAT | W | 112 | 67.813 | 81.789 | −25.634 | 1.00 | 15.49 | W |
| ATOM | 8295 | OH2 | WAT | W | 113 | 22.910 | 51.833 | −8.728 | 1.00 | 17.22 | W |
| ATOM | 8296 | OH2 | WAT | W | 114 | 46.597 | 70.553 | −36.714 | 1.00 | 16.13 | W |
| ATOM | 8297 | OH2 | WAT | W | 115 | 20.543 | 57.579 | −5.285 | 1.00 | 11.86 | W |
| ATOM | 8298 | OH2 | WAT | W | 116 | 18.154 | 60.637 | 24.428 | 1.00 | 23.17 | W |
| ATOM | 8299 | OH2 | WAT | W | 117 | 41.280 | 68.626 | −32.512 | 1.00 | 16.76 | W |
| ATOM | 8300 | OH2 | WAT | W | 118 | 38.328 | 40.122 | 1.665 | 1.00 | 12.98 | W |
| ATOM | 8301 | OH2 | WAT | W | 119 | 23.855 | 58.244 | 6.312 | 1.00 | 12.38 | W |
| ATOM | 8302 | OH2 | WAT | W | 120 | 18.101 | 59.927 | −20.092 | 1.00 | 23.40 | W |
| ATOM | 8303 | OH2 | WAT | W | 121 | 41.276 | 77.738 | −14.102 | 1.00 | 15.70 | W |
| ATOM | 8304 | OH2 | WAT | W | 122 | 52.842 | 59.201 | −1.699 | 1.00 | 21.35 | W |
| ATOM | 8305 | OH2 | WAT | W | 123 | 47.942 | 49.607 | 13.775 | 1.00 | 12.65 | W |
| ATOM | 8306 | OH2 | WAT | W | 124 | 35.232 | 43.720 | 28.641 | 1.00 | 15.37 | W |
| ATOM | 8307 | OH2 | WAT | W | 125 | 59.499 | 60.738 | −6.298 | 1.00 | 12.21 | W |
| ATOM | 8308 | OH2 | WAT | W | 126 | 53.661 | 72.740 | −19.148 | 1.00 | 13.77 | W |
| ATOM | 8309 | OH2 | WAT | W | 127 | 39.796 | 54.103 | −20.838 | 1.00 | 20.47 | W |
| ATOM | 8310 | OH2 | WAT | W | 128 | 33.113 | 36.777 | −4.404 | 1.00 | 19.85 | W |
| ATOM | 8311 | OH2 | WAT | W | 129 | 14.324 | 64.079 | −16.361 | 1.00 | 16.40 | W |
| ATOM | 8312 | OH2 | WAT | W | 130 | 47.511 | 43.081 | −2.434 | 1.00 | 16.15 | W |
| ATOM | 8313 | OH2 | WAT | W | 131 | 46.904 | 56.002 | −25.485 | 1.00 | 17.18 | W |
| ATOM | 8314 | OH2 | WAT | W | 132 | 32.138 | 58.794 | −28.468 | 1.00 | 18.35 | W |
| ATOM | 8315 | OH2 | WAT | W | 133 | 49.264 | 50.050 | 28.230 | 1.00 | 22.47 | W |
| ATOM | 8316 | OH2 | WAT | W | 134 | 26.292 | 37.411 | 29.332 | 1.00 | 24.20 | W |
| ATOM | 8317 | OH2 | WAT | W | 135 | 51.883 | 46.667 | 1.206 | 1.00 | 12.72 | W |
| ATOM | 8318 | OH2 | WAT | W | 136 | 41.713 | 58.870 | −16.022 | 1.00 | 14.58 | W |
| ATOM | 8319 | OH2 | WAT | W | 137 | 13.817 | 54.250 | 4.446 | 1.00 | 20.80 | W |
| ATOM | 8320 | OH2 | WAT | W | 138 | 67.797 | 79.183 | −25.331 | 1.00 | 15.35 | W |
| ATOM | 8321 | OH2 | WAT | W | 139 | 42.168 | 75.429 | −16.021 | 1.00 | 16.13 | W |
| ATOM | 8322 | OH2 | WAT | W | 140 | 23.119 | 56.194 | 30.804 | 1.00 | 18.52 | W |
| ATOM | 8323 | OH2 | WAT | W | 141 | 56.988 | 61.152 | 1.554 | 1.00 | 17.70 | W |
| ATOM | 8324 | OH2 | WAT | W | 142 | 20.075 | 74.547 | −6.879 | 1.00 | 14.89 | W |
| ATOM | 8325 | OH2 | WAT | W | 143 | 13.378 | 53.661 | 1.806 | 1.00 | 17.64 | W |
| ATOM | 8326 | OH2 | WAT | W | 144 | 34.280 | 34.507 | 16.087 | 1.00 | 20.88 | W |
| ATOM | 8327 | OH2 | WAT | W | 145 | 33.932 | 64.705 | −32.236 | 1.00 | 16.75 | W |
| ATOM | 8328 | OH2 | WAT | W | 146 | 14.061 | 49.293 | 15.438 | 1.00 | 16.25 | W |
| ATOM | 8329 | OH2 | WAT | W | 147 | 30.384 | 35.313 | −12.407 | 1.00 | 20.29 | W |
| ATOM | 8330 | OH2 | WAT | W | 148 | 18.403 | 55.006 | 14.038 | 1.00 | 14.99 | W |
| ATOM | 8331 | OH2 | WAT | W | 149 | 15.636 | 60.472 | 15.465 | 1.00 | 21.47 | W |
| ATOM | 8332 | OH2 | WAT | W | 150 | 16.791 | 74.875 | −3.047 | 1.00 | 20.80 | W |
| ATOM | 8333 | OH2 | WAT | W | 151 | 56.518 | 46.319 | −17.443 | 1.00 | 19.10 | W |
| ATOM | 8334 | OH2 | WAT | W | 152 | 45.628 | 60.100 | −18.241 | 1.00 | 16.63 | W |
| ATOM | 8335 | OH2 | WAT | W | 153 | 28.171 | 84.777 | −35.426 | 1.00 | 18.35 | W |
| ATOM | 8336 | OH2 | WAT | W | 154 | 73.029 | 65.246 | −7.720 | 1.00 | 18.19 | W |
| ATOM | 8337 | OH2 | WAT | W | 155 | 73.770 | 66.211 | −17.640 | 1.00 | 18.74 | W |
| ATOM | 8338 | OH2 | WAT | W | 156 | 59.259 | 50.047 | −0.667 | 1.00 | 17.63 | W |
| ATOM | 8339 | OH2 | WAT | W | 157 | 23.044 | 78.525 | −24.838 | 1.00 | 20.25 | W |
| ATOM | 8340 | OH2 | WAT | W | 158 | 14.298 | 54.500 | −16.068 | 1.00 | 18.13 | W |
| ATOM | 8341 | OH2 | WAT | W | 159 | 43.558 | 73.338 | 5.809 | 1.00 | 12.80 | W |
| ATOM | 8342 | OH2 | WAT | W | 160 | 11.895 | 55.194 | −12.525 | 1.00 | 18.10 | W |
| ATOM | 8343 | OH2 | WAT | W | 161 | 63.550 | 44.738 | −6.929 | 1.00 | 16.10 | W |
| ATOM | 8344 | OH2 | WAT | W | 162 | 24.753 | 51.132 | −24.121 | 1.00 | 17.61 | W |
| ATOM | 8345 | OH2 | WAT | W | 163 | 35.486 | 45.424 | −27.380 | 1.00 | 17.74 | W |
| ATOM | 8346 | OH2 | WAT | W | 164 | 28.688 | 49.888 | −24.414 | 1.00 | 21.74 | W |
| ATOM | 8347 | OH2 | WAT | W | 165 | 54.813 | 81.192 | −23.474 | 1.00 | 17.16 | W |
| ATOM | 8348 | OH2 | WAT | W | 166 | 23.604 | 63.804 | 7.820 | 1.00 | 20.53 | W |
| ATOM | 8349 | OH2 | WAT | W | 167 | 49.744 | 57.760 | −7.648 | 1.00 | 12.03 | W |
| ATOM | 8350 | OH2 | WAT | W | 168 | 46.061 | 71.148 | 0.115 | 1.00 | 20.51 | W |
| ATOM | 8351 | OH2 | WAT | W | 169 | 24.323 | 55.822 | −1.895 | 1.00 | 14.21 | W |
| ATOM | 8352 | OH2 | WAT | W | 170 | 28.647 | 41.366 | −21.778 | 1.00 | 15.50 | W |
| ATOM | 8353 | OH2 | WAT | W | 171 | 37.052 | 45.730 | 27.466 | 1.00 | 15.23 | W |
| ATOM | 8354 | OH2 | WAT | W | 172 | 41.487 | 41.345 | −17.622 | 1.00 | 17.68 | W |
| ATOM | 8355 | OH2 | WAT | W | 173 | 40.305 | 62.726 | −31.970 | 1.00 | 17.43 | W |
| ATOM | 8356 | OH2 | WAT | W | 174 | 49.313 | 59.380 | −28.687 | 1.00 | 16.29 | W |
| ATOM | 8357 | OH2 | WAT | W | 175 | 64.386 | 39.376 | −10.440 | 1.00 | 28.13 | W |
| ATOM | 8358 | OH2 | WAT | W | 176 | 19.168 | 72.621 | 2.908 | 1.00 | 21.55 | W |
| ATOM | 8359 | OH2 | WAT | W | 177 | 17.064 | 70.461 | −16.407 | 1.00 | 15.86 | W |
| ATOM | 8360 | OH2 | WAT | W | 178 | 9.518 | 52.618 | 16.850 | 1.00 | 29.64 | W |
| ATOM | 8361 | OH2 | WAT | W | 179 | 53.879 | 71.479 | −36.735 | 1.00 | 13.71 | W |
| ATOM | 8362 | OH2 | WAT | W | 180 | 50.889 | 71.054 | −23.486 | 1.00 | 16.09 | W |
| ATOM | 8363 | OH2 | WAT | W | 181 | 49.384 | 44.309 | 4.445 | 1.00 | 15.10 | W |
| ATOM | 8364 | OH2 | WAT | W | 182 | 59.367 | 68.444 | 2.370 | 1.00 | 21.67 | W |
| ATOM | 8365 | OH2 | WAT | W | 183 | 25.439 | 53.900 | 11.086 | 1.00 | 15.64 | W |
| ATOM | 8366 | OH2 | WAT | W | 184 | 69.086 | 61.568 | −2.411 | 1.00 | 16.23 | W |
| ATOM | 8367 | OH2 | WAT | W | 185 | 58.341 | 77.603 | −38.440 | 1.00 | 18.02 | W |
| ATOM | 8368 | OH2 | WAT | W | 186 | 44.390 | 46.220 | 9.936 | 1.00 | 17.37 | W |
| ATOM | 8369 | OH2 | WAT | W | 187 | 46.547 | 57.491 | −18.779 | 1.00 | 13.56 | W |
| ATOM | 8370 | OH2 | WAT | W | 188 | 33.493 | 83.726 | −10.384 | 1.00 | 16.90 | W |
| ATOM | 8371 | OH2 | WAT | W | 189 | 47.052 | 68.688 | −30.395 | 1.00 | 20.70 | W |
| ATOM | 8372 | OH2 | WAT | W | 190 | 44.386 | 56.163 | −15.436 | 1.00 | 17.54 | W |
| ATOM | 8373 | OH2 | WAT | W | 191 | 13.141 | 67.021 | −4.770 | 1.00 | 16.21 | W |
| ATOM | 8374 | OH2 | WAT | W | 192 | 24.512 | 39.271 | −11.474 | 1.00 | 19.84 | W |
| ATOM | 8375 | OH2 | WAT | W | 193 | 41.591 | 61.284 | −29.731 | 1.00 | 17.96 | W |
| ATOM | 8376 | OH2 | WAT | W | 194 | 27.187 | 41.479 | 13.299 | 1.00 | 18.70 | W |
| ATOM | 8377 | OH2 | WAT | W | 195 | 42.003 | 88.025 | −40.833 | 1.00 | 20.34 | W |
| ATOM | 8378 | OH2 | WAT | W | 196 | 69.850 | 70.102 | −12.637 | 1.00 | 18.95 | W |
| ATOM | 8379 | OH2 | WAT | W | 197 | 64.133 | 78.095 | −35.174 | 1.00 | 15.70 | W |
| ATOM | 8380 | OH2 | WAT | W | 198 | 20.411 | 65.476 | 7.641 | 1.00 | 22.81 | W |
| ATOM | 8381 | OH2 | WAT | W | 199 | 11.382 | 61.518 | −18.592 | 1.00 | 24.08 | W |
| ATOM | 8382 | OH2 | WAT | W | 200 | 24.515 | 70.804 | −8.824 | 1.00 | 12.95 | W |
| ATOM | 8383 | OH2 | WAT | W | 201 | 25.028 | 40.143 | 14.192 | 1.00 | 15.50 | W |
| ATOM | 8384 | OH2 | WAT | W | 202 | 22.728 | 63.442 | −33.182 | 1.00 | 19.12 | W |
| ATOM | 8385 | OH2 | WAT | W | 203 | 41.675 | 43.431 | 7.944 | 1.00 | 16.39 | W |
| ATOM | 8386 | OH2 | WAT | W | 204 | 21.035 | 51.916 | 47.273 | 1.00 | 21.06 | W |
| ATOM | 8387 | OH2 | WAT | W | 205 | 41.323 | 50.434 | 33.218 | 1.00 | 21.10 | W |
| ATOM | 8388 | OH2 | WAT | W | 206 | 45.961 | 52.944 | −19.253 | 1.00 | 20.91 | W |
| ATOM | 8389 | OH2 | WAT | W | 207 | 51.427 | 76.534 | −40.959 | 1.00 | 29.19 | W |
| ATOM | 8390 | OH2 | WAT | W | 208 | 25.701 | 86.532 | −31.930 | 1.00 | 16.19 | W |
| ATOM | 8391 | OH2 | WAT | W | 209 | 12.460 | 56.457 | 5.406 | 1.00 | 18.11 | W |
| ATOM | 8392 | OH2 | WAT | W | 210 | 22.528 | 56.988 | −34.123 | 1.00 | 19.06 | W |
| ATOM | 8393 | OH2 | WAT | W | 211 | 43.856 | 72.929 | −42.044 | 1.00 | 28.46 | W |
| ATOM | 8394 | OH2 | WAT | W | 212 | 44.594 | 58.887 | −16.004 | 1.00 | 14.67 | W |
| ATOM | 8395 | OH2 | WAT | W | 213 | 31.327 | 81.894 | 0.496 | 1.00 | 18.34 | W |
| ATOM | 8396 | OH2 | WAT | W | 214 | 51.990 | 56.524 | −30.250 | 1.00 | 22.59 | W |
| ATOM | 8397 | OH2 | WAT | W | 215 | 17.291 | 70.212 | −23.398 | 1.00 | 26.14 | W |
| ATOM | 8398 | OH2 | WAT | W | 216 | 37.674 | 47.140 | 29.767 | 1.00 | 18.64 | W |
| ATOM | 8399 | OH2 | WAT | W | 217 | 49.763 | 55.189 | −28.946 | 1.00 | 20.75 | W |
| ATOM | 8400 | OH2 | WAT | W | 218 | 36.224 | 79.442 | −5.010 | 1.00 | 20.29 | W |
| ATOM | 8401 | OH2 | WAT | W | 219 | 62.253 | 62.767 | 25.731 | 1.00 | 18.51 | W |
| ATOM | 8402 | OH2 | WAT | W | 220 | 11.367 | 53.252 | 6.826 | 1.00 | 16.35 | W |
| ATOM | 8403 | OH2 | WAT | W | 221 | 13.918 | 60.924 | −22.492 | 1.00 | 17.57 | W |
| ATOM | 8404 | OH2 | WAT | W | 222 | 50.826 | 62.132 | 26.293 | 1.00 | 18.66 | W |
| ATOM | 8405 | OH2 | WAT | W | 223 | 68.567 | 79.132 | −18.104 | 1.00 | 23.07 | W |
| ATOM | 8406 | OH2 | WAT | W | 224 | 84.246 | 68.535 | −17.436 | 1.00 | 18.57 | W |
| ATOM | 8407 | OH2 | WAT | W | 225 | 21.706 | 40.481 | −8.839 | 1.00 | 39.04 | W |
| ATOM | 8408 | OH2 | WAT | W | 226 | 52.774 | 69.943 | 18.294 | 1.00 | 29.32 | W |
| ATOM | 8409 | OH2 | WAT | W | 227 | 42.710 | 53.366 | 35.547 | 1.00 | 23.23 | W |
| ATOM | 8410 | OH2 | WAT | W | 228 | 70.573 | 52.241 | −12.697 | 1.00 | 18.76 | W |
| ATOM | 8411 | OH2 | WAT | W | 229 | 34.677 | 70.519 | 13.163 | 1.00 | 15.62 | W |
| ATOM | 8412 | OH2 | WAT | W | 230 | 27.183 | 46.437 | 13.610 | 1.00 | 17.02 | W |
| ATOM | 8413 | OH2 | WAT | W | 231 | 14.461 | 49.789 | 19.876 | 1.00 | 21.18 | W |
| ATOM | 8414 | OH2 | WAT | W | 232 | 49.677 | 73.603 | −21.812 | 1.00 | 20.74 | W |
| ATOM | 8415 | OH2 | WAT | W | 233 | 48.689 | 44.397 | −6.207 | 1.00 | 23.27 | W |
| ATOM | 8416 | OH2 | WAT | W | 234 | 61.411 | 77.066 | −39.605 | 1.00 | 20.10 | W |
| ATOM | 8417 | OH2 | WAT | W | 235 | 22.867 | 78.771 | −30.351 | 1.00 | 27.37 | W |
| ATOM | 8418 | OH2 | WAT | W | 236 | 43.608 | 88.812 | −38.512 | 1.00 | 22.55 | W |
| ATOM | 8419 | OH2 | WAT | W | 237 | 35.757 | 77.729 | −7.317 | 1.00 | 14.57 | W |
| ATOM | 8420 | OH2 | WAT | W | 238 | 24.900 | 68.466 | 21.787 | 1.00 | 17.96 | W |
| ATOM | 8421 | OH2 | WAT | W | 239 | 68.063 | 49.283 | −1.100 | 1.00 | 22.72 | W |
| ATOM | 8422 | OH2 | WAT | W | 240 | 54.299 | 48.026 | 15.591 | 1.00 | 25.88 | W |
| ATOM | 8423 | OH2 | WAT | W | 241 | 55.049 | 50.600 | 16.690 | 1.00 | 28.67 | W |
| ATOM | 8424 | OH2 | WAT | W | 242 | 39.361 | 48.842 | −32.770 | 1.00 | 25.18 | W |
| ATOM | 8425 | OH2 | WAT | W | 243 | 22.699 | 45.380 | −13.467 | 1.00 | 21.72 | W |
| ATOM | 8426 | OH2 | WAT | W | 244 | 67.040 | 55.442 | 0.924 | 1.00 | 15.31 | W |
| ATOM | 8427 | OH2 | WAT | W | 245 | 47.931 | 44.056 | −8.657 | 1.00 | 22.96 | W |
| ATOM | 8428 | OH2 | WAT | W | 246 | 26.926 | 87.957 | −23.673 | 1.00 | 20.73 | W |
| ATOM | 8429 | OH2 | WAT | W | 247 | 18.939 | 48.658 | 37.726 | 1.00 | 21.79 | W |
| ATOM | 8430 | OH2 | WAT | W | 248 | 83.613 | 67.735 | −22.892 | 1.00 | 25.89 | W |
| ATOM | 8431 | OH2 | WAT | W | 249 | 43.080 | 59.956 | −19.011 | 1.00 | 22.21 | W |
| ATOM | 8432 | OH2 | WAT | W | 250 | 47.447 | 79.280 | −40.381 | 1.00 | 21.91 | W |
| ATOM | 8433 | OH2 | WAT | W | 251 | 8.610 | 57.762 | −6.870 | 1.00 | 27.88 | W |
| ATOM | 8434 | OH2 | WAT | W | 252 | 54.864 | 90.452 | −23.484 | 1.00 | 28.07 | W |
| ATOM | 8435 | OH2 | WAT | W | 253 | 27.829 | 36.704 | −19.683 | 1.00 | 24.79 | W |
| ATOM | 8436 | OH2 | WAT | W | 254 | 72.041 | 58.809 | −11.211 | 1.00 | 23.82 | W |
| ATOM | 8437 | OH2 | WAT | W | 255 | 34.317 | 67.941 | 12.414 | 1.00 | 12.73 | W |
| ATOM | 8438 | OH2 | WAT | W | 256 | 11.689 | 59.501 | −4.979 | 1.00 | 21.37 | W |
| ATOM | 8439 | OH2 | WAT | W | 257 | 23.547 | 46.199 | −15.862 | 1.00 | 24.69 | W |
| ATOM | 8440 | OH2 | WAT | W | 258 | 50.148 | 70.938 | −17.614 | 1.00 | 19.04 | W |
| ATOM | 8441 | OH2 | WAT | W | 259 | 45.026 | 90.336 | −25.359 | 1.00 | 23.05 | W |
| ATOM | 8442 | OH2 | WAT | W | 260 | 16.051 | 72.410 | −19.141 | 1.00 | 18.99 | W |
| ATOM | 8443 | OH2 | WAT | W | 261 | 20.057 | 52.682 | −24.371 | 1.00 | 18.41 | W |
| ATOM | 8444 | OH2 | WAT | W | 262 | 59.525 | 76.638 | −8.915 | 1.00 | 20.35 | W |
| ATOM | 8445 | OH2 | WAT | W | 263 | 67.003 | 60.112 | −27.213 | 1.00 | 21.44 | W |
| ATOM | 8446 | OH2 | WAT | W | 264 | 38.567 | 51.573 | −29.906 | 1.00 | 19.02 | W |
| ATOM | 8447 | OH2 | WAT | W | 265 | 40.324 | 83.008 | −10.715 | 1.00 | 18.13 | W |
| ATOM | 8448 | OH2 | WAT | W | 266 | 42.243 | 48.804 | 31.283 | 1.00 | 18.64 | W |
| ATOM | 8449 | OH2 | WAT | W | 267 | 24.084 | 53.092 | −6.116 | 1.00 | 17.16 | W |
| ATOM | 8450 | OH2 | WAT | W | 268 | 53.144 | 47.239 | −24.933 | 1.00 | 20.15 | W |
| ATOM | 8451 | OH2 | WAT | W | 269 | 32.591 | 89.326 | −43.014 | 1.00 | 25.83 | W |
| ATOM | 8452 | OH2 | WAT | W | 270 | 35.918 | 36.131 | −4.652 | 1.00 | 19.94 | W |
| ATOM | 8453 | OH2 | WAT | W | 271 | 70.097 | 83.236 | −29.819 | 1.00 | 25.99 | W |
| ATOM | 8454 | OH2 | WAT | W | 272 | 49.672 | 82.609 | −43.732 | 1.00 | 22.21 | W |
| ATOM | 8455 | OH2 | WAT | W | 273 | 68.303 | 79.781 | −34.802 | 1.00 | 25.72 | W |
| ATOM | 8456 | OH2 | WAT | W | 274 | 29.275 | 46.925 | 37.053 | 1.00 | 24.15 | W |
| ATOM | 8457 | OH2 | WAT | W | 275 | 18.487 | 70.059 | 4.190 | 1.00 | 14.14 | W |
| ATOM | 8458 | OH2 | WAT | W | 276 | 14.136 | 68.374 | −7.960 | 1.00 | 20.02 | W |
| ATOM | 8459 | OH2 | WAT | W | 277 | 50.303 | 72.617 | 9.138 | 1.00 | 33.87 | W |
| ATOM | 8460 | OH2 | WAT | W | 278 | 39.685 | 75.893 | −40.177 | 1.00 | 20.18 | W |
| ATOM | 8461 | OH2 | WAT | W | 279 | 28.798 | 65.257 | 19.691 | 1.00 | 15.58 | W |
| ATOM | 8462 | OH2 | WAT | W | 280 | 11.686 | 58.802 | 7.875 | 1.00 | 22.13 | W |
| ATOM | 8463 | OH2 | WAT | W | 281 | 52.678 | 79.065 | −17.312 | 1.00 | 23.55 | W |
| ATOM | 8464 | OH2 | WAT | W | 282 | 18.551 | 65.160 | −27.061 | 1.00 | 24.87 | W |
| ATOM | 8465 | OH2 | WAT | W | 283 | 44.655 | 79.479 | −33.163 | 1.00 | 23.28 | W |
| ATOM | 8466 | OH2 | WAT | W | 284 | 36.141 | 81.260 | 6.293 | 1.00 | 20.52 | W |
| ATOM | 8467 | OH2 | WAT | W | 285 | 16.704 | 60.781 | −28.283 | 1.00 | 26.21 | W |
| ATOM | 8468 | OH2 | WAT | W | 286 | 29.546 | 87.479 | −17.987 | 1.00 | 29.41 | W |
| ATOM | 8469 | OH2 | WAT | W | 287 | 8.873 | 55.123 | −6.136 | 1.00 | 25.16 | W |
| ATOM | 8470 | OH2 | WAT | W | 288 | 46.657 | 61.790 | −25.940 | 1.00 | 21.32 | W |
| ATOM | 8471 | OH2 | WAT | W | 289 | 42.933 | 82.357 | −10.072 | 1.00 | 30.75 | W |
| ATOM | 8472 | OH2 | WAT | W | 290 | 10.304 | 50.828 | 18.854 | 1.00 | 22.62 | W |
| ATOM | 8473 | OH2 | WAT | W | 291 | 12.803 | 42.588 | 12.658 | 1.00 | 17.50 | W |
| ATOM | 8474 | OH2 | WAT | W | 292 | 35.953 | 45.284 | 30.852 | 1.00 | 21.96 | W |
| ATOM | 8475 | OH2 | WAT | W | 293 | 15.695 | 75.427 | −11.773 | 1.00 | 28.82 | W |
| ATOM | 8476 | OH2 | WAT | W | 294 | 25.241 | 46.933 | −20.851 | 1.00 | 26.50 | W |
| ATOM | 8477 | OH2 | WAT | W | 295 | 56.985 | 65.097 | 24.144 | 1.00 | 25.11 | W |
| ATOM | 8478 | OH2 | WAT | W | 296 | 38.666 | 94.400 | −42.035 | 1.00 | 24.62 | W |
| ATOM | 8479 | OH2 | WAT | W | 297 | 34.710 | 59.743 | −34.933 | 1.00 | 22.62 | W |
| ATOM | 8480 | OH2 | WAT | W | 298 | 32.944 | 41.331 | −17.505 | 1.00 | 26.13 | W |
| ATOM | 8481 | OH2 | WAT | W | 299 | 20.657 | 42.768 | −7.509 | 1.00 | 21.95 | W |
| ATOM | 8482 | OH2 | WAT | W | 300 | 19.155 | 76.985 | 4.910 | 1.00 | 38.11 | W |
| ATOM | 8483 | OH2 | WAT | W | 301 | 43.373 | 80.840 | 4.965 | 1.00 | 24.73 | W |
| ATOM | 8484 | OH2 | WAT | W | 302 | 32.881 | 85.482 | −5.464 | 1.00 | 21.36 | W |
| ATOM | 8485 | OH2 | WAT | W | 303 | 31.104 | 33.601 | 2.777 | 1.00 | 24.21 | W |
| ATOM | 8486 | OH2 | WAT | W | 304 | 21.396 | 78.741 | −1.313 | 1.00 | 21.96 | W |
| ATOM | 8487 | OH2 | WAT | W | 305 | 55.254 | 59.216 | 4.036 | 1.00 | 21.45 | W |
| ATOM | 8488 | OH2 | WAT | W | 306 | 13.447 | 62.535 | −19.982 | 1.00 | 20.72 | W |
| ATOM | 8489 | OH2 | WAT | W | 307 | 16.955 | 51.042 | −19.284 | 1.00 | 29.66 | W |
| ATOM | 8490 | OH2 | WAT | W | 308 | 14.307 | 62.996 | −12.146 | 1.00 | 18.26 | W |
| ATOM | 8491 | OH2 | WAT | W | 309 | 53.317 | 86.994 | −21.280 | 1.00 | 26.84 | W |
| ATOM | 8492 | OH2 | WAT | W | 310 | 70.858 | 49.588 | −0.140 | 1.00 | 24.53 | W |
| ATOM | 8493 | OH2 | WAT | W | 311 | 57.961 | 42.561 | 1.493 | 1.00 | 26.85 | W |
| ATOM | 8494 | OH2 | WAT | W | 312 | 74.694 | 76.842 | −14.248 | 1.00 | 29.16 | W |
| ATOM | 8495 | OH2 | WAT | W | 313 | 24.860 | 83.244 | −16.897 | 1.00 | 20.51 | W |
| ATOM | 8496 | OH2 | WAT | W | 314 | 56.992 | 67.918 | −35.378 | 1.00 | 23.70 | W |
| ATOM | 8497 | OH2 | WAT | W | 315 | 48.877 | 50.823 | −29.576 | 1.00 | 27.22 | W |
| ATOM | 8498 | OH2 | WAT | W | 316 | 66.517 | 71.811 | −6.772 | 1.00 | 20.99 | W |
| ATOM | 8499 | OH2 | WAT | W | 317 | 28.748 | 93.976 | −38.371 | 1.00 | 26.19 | W |
| ATOM | 8500 | OH2 | WAT | W | 318 | 32.645 | 64.843 | −10.052 | 1.00 | 16.14 | W |
| ATOM | 8501 | OH2 | WAT | W | 319 | 13.378 | 51.128 | −1.741 | 1.00 | 26.12 | W |
| ATOM | 8502 | OH2 | WAT | W | 320 | 39.215 | 49.398 | 30.134 | 1.00 | 22.80 | W |
| ATOM | 8503 | OH2 | WAT | W | 321 | 67.798 | 58.798 | −23.988 | 1.00 | 20.43 | W |
| ATOM | 8504 | OH2 | WAT | W | 322 | 20.725 | 59.171 | −34.517 | 1.00 | 19.35 | W |
| ATOM | 8505 | OH2 | WAT | W | 323 | 37.620 | 75.876 | −42.374 | 1.00 | 22.17 | W |
| ATOM | 8506 | OH2 | WAT | W | 324 | 31.748 | 51.402 | −35.453 | 1.00 | 25.15 | W |
| ATOM | 8507 | OH2 | WAT | W | 325 | 13.024 | 71.443 | −0.523 | 1.00 | 29.86 | W |
| ATOM | 8508 | OH2 | WAT | W | 326 | 36.937 | 41.116 | −24.855 | 1.00 | 27.46 | W |
| ATOM | 8509 | OH2 | WAT | W | 327 | 19.245 | 42.686 | −2.510 | 1.00 | 29.66 | W |
| ATOM | 8510 | OH2 | WAT | W | 328 | 49.929 | 44.230 | 7.118 | 1.00 | 23.60 | W |
| ATOM | 8511 | OH2 | WAT | W | 329 | 37.331 | 45.560 | −25.526 | 1.00 | 26.02 | W |
| ATOM | 8512 | OH2 | WAT | W | 330 | 75.334 | 74.276 | −19.598 | 1.00 | 24.04 | W |
| ATOM | 8513 | OH2 | WAT | W | 331 | 50.302 | 61.343 | −31.180 | 1.00 | 20.21 | W |
| ATOM | 8514 | OH2 | WAT | W | 332 | 42.966 | 56.923 | −31.510 | 1.00 | 28.63 | W |
| ATOM | 8515 | OH2 | WAT | W | 333 | 13.218 | 68.993 | 0.963 | 1.00 | 23.46 | W |
| ATOM | 8516 | OH2 | WAT | W | 334 | 40.987 | 83.074 | −31.356 | 1.00 | 27.25 | W |
| ATOM | 8517 | OH2 | WAT | W | 335 | 26.940 | 52.514 | 22.434 | 1.00 | 28.73 | W |
| ATOM | 8518 | OH2 | WAT | W | 336 | 12.531 | 66.524 | −9.404 | 1.00 | 21.80 | W |
| ATOM | 8519 | OH2 | WAT | W | 337 | 42.599 | 39.444 | −15.832 | 1.00 | 27.01 | W |
| ATOM | 8520 | OH2 | WAT | W | 338 | 48.048 | 48.441 | 16.333 | 1.00 | 19.47 | W |
| ATOM | 8521 | OH2 | WAT | W | 339 | 11.378 | 46.854 | 17.807 | 1.00 | 30.09 | W |
| ATOM | 8522 | OH2 | WAT | W | 340 | 16.441 | 67.947 | −19.971 | 1.00 | 21.87 | W |
| ATOM | 8523 | OH2 | WAT | W | 341 | 5.169 | 52.976 | −6.017 | 1.00 | 34.52 | W |
| ATOM | 8524 | OH2 | WAT | W | 342 | 56.366 | 91.394 | −25.691 | 1.00 | 22.25 | W |
| ATOM | 8525 | OH2 | WAT | W | 343 | 39.138 | 83.536 | −27.276 | 1.00 | 21.07 | W |
| ATOM | 8526 | OH2 | WAT | W | 344 | 60.932 | 57.026 | 15.691 | 1.00 | 33.10 | W |
| ATOM | 8527 | OH2 | WAT | W | 345 | 13.476 | 51.603 | 21.683 | 1.00 | 23.52 | W |
| ATOM | 8528 | OH2 | WAT | W | 346 | 43.478 | 94.149 | −37.697 | 1.00 | 26.37 | W |
| ATOM | 8529 | OH2 | WAT | W | 347 | 28.012 | 35.395 | −6.915 | 1.00 | 24.58 | W |
| ATOM | 8530 | OH2 | WAT | W | 348 | 80.215 | 64.256 | −21.465 | 1.00 | 25.69 | W |
| ATOM | 8531 | OH2 | WAT | W | 349 | 51.571 | 45.254 | 9.731 | 1.00 | 32.98 | W |
| ATOM | 8532 | OH2 | WAT | W | 350 | 21.425 | 46.768 | −17.469 | 1.00 | 30.25 | W |
| ATOM | 8533 | OH2 | WAT | W | 351 | 47.390 | 72.020 | −20.194 | 1.00 | 22.19 | W |
| ATOM | 8534 | OH2 | WAT | W | 352 | 21.553 | 86.243 | −30.890 | 1.00 | 31.55 | W |
| ATOM | 8535 | OH2 | WAT | W | 353 | 33.014 | 67.065 | 21.351 | 1.00 | 20.88 | W |
| ATOM | 8536 | OH2 | WAT | W | 354 | 14.654 | 62.181 | −14.551 | 1.00 | 20.03 | W |
| ATOM | 8537 | OH2 | WAT | W | 355 | 50.342 | 73.852 | −25.133 | 1.00 | 24.70 | W |
| ATOM | 8538 | OH2 | WAT | W | 356 | 41.982 | 79.510 | −23.663 | 1.00 | 24.72 | W |
| ATOM | 8539 | OH2 | WAT | W | 357 | 39.987 | 45.435 | 25.472 | 1.00 | 27.22 | W |
| ATOM | 8540 | OH2 | WAT | W | 358 | 50.966 | 76.620 | 14.400 | 1.00 | 25.22 | W |
| ATOM | 8541 | OH2 | WAT | W | 359 | 38.560 | 45.243 | −33.596 | 1.00 | 23.12 | W |
| ATOM | 8542 | OH2 | WAT | W | 360 | 61.497 | 54.297 | −28.826 | 1.00 | 32.75 | W |
| ATOM | 8543 | OH2 | WAT | W | 361 | 53.745 | 46.872 | 23.471 | 1.00 | 27.63 | W |
| ATOM | 8544 | OH2 | WAT | W | 362 | 57.002 | 43.240 | −20.162 | 1.00 | 26.84 | W |
| ATOM | 8545 | OH2 | WAT | W | 363 | 67.620 | 54.903 | −18.283 | 1.00 | 25.83 | W |
| ATOM | 8546 | OH2 | WAT | W | 364 | 42.433 | 80.253 | −31.603 | 1.00 | 35.61 | W |
| ATOM | 8547 | OH2 | WAT | W | 365 | 29.079 | 62.396 | −39.782 | 1.00 | 28.70 | W |
| ATOM | 8548 | OH2 | WAT | W | 366 | 26.835 | 61.857 | 10.737 | 1.00 | 29.62 | W |
| ATOM | 8549 | OH2 | WAT | W | 367 | 45.820 | 45.105 | 22.830 | 1.00 | 32.71 | W |
| ATOM | 8550 | OH2 | WAT | W | 368 | 35.144 | 51.275 | 35.780 | 1.00 | 30.60 | W |
| ATOM | 8551 | OH2 | WAT | W | 369 | 57.657 | 62.439 | 7.864 | 1.00 | 30.02 | W |
| ATOM | 8552 | OH2 | WAT | W | 370 | 25.335 | 33.634 | 13.186 | 1.00 | 24.09 | W |
| ATOM | 8553 | OH2 | WAT | W | 371 | 27.668 | 59.493 | 8.703 | 1.00 | 24.24 | W |
| ATOM | 8554 | OH2 | WAT | W | 372 | 42.896 | 94.777 | −30.168 | 1.00 | 42.53 | W |
| ATOM | 8555 | OH2 | WAT | W | 373 | 13.858 | 58.126 | −1.438 | 1.00 | 27.82 | W |
| ATOM | 8556 | OH2 | WAT | W | 374 | 49.215 | 76.579 | 5.508 | 1.00 | 27.65 | W |
| ATOM | 8557 | OH2 | WAT | W | 375 | 27.926 | 67.675 | 28.190 | 1.00 | 26.72 | W |
| ATOM | 8558 | OH2 | WAT | W | 376 | 41.928 | 39.313 | 16.708 | 1.00 | 34.69 | W |
| ATOM | 8559 | OH2 | WAT | W | 377 | 58.774 | 46.538 | −1.484 | 1.00 | 18.42 | W |
| ATOM | 8560 | OH2 | WAT | W | 378 | 73.332 | 76.951 | −18.539 | 1.00 | 31.21 | W |
| ATOM | 8561 | OH2 | WAT | W | 379 | 19.667 | 39.457 | 17.556 | 1.00 | 20.16 | W |
| ATOM | 8562 | OH2 | WAT | W | 380 | 39.703 | 59.128 | −18.068 | 1.00 | 17.51 | W |
| ATOM | 8563 | OH2 | WAT | W | 381 | 28.065 | 47.264 | −31.490 | 1.00 | 27.85 | W |
| ATOM | 8564 | OH2 | WAT | W | 382 | 68.593 | 47.077 | −16.564 | 1.00 | 25.08 | W |
| ATOM | 8565 | OH2 | WAT | W | 383 | 66.499 | 62.814 | −13.279 | 1.00 | 22.18 | W |
| ATOM | 8566 | OH2 | WAT | W | 384 | 26.536 | 75.252 | 2.031 | 1.00 | 20.08 | W |
| ATOM | 8567 | OH2 | WAT | W | 385 | 39.980 | 39.135 | 9.175 | 1.00 | 29.93 | W |
| ATOM | 8568 | OH2 | WAT | W | 386 | 21.531 | 47.191 | 39.117 | 1.00 | 28.17 | W |
| ATOM | 8569 | OH2 | WAT | W | 387 | 42.190 | 75.026 | −41.376 | 1.00 | 29.68 | W |
| ATOM | 8570 | OH2 | WAT | W | 388 | 14.674 | 55.669 | 27.161 | 1.00 | 33.37 | W |
| ATOM | 8571 | OH2 | WAT | W | 389 | 28.615 | 59.877 | −39.093 | 1.00 | 28.02 | W |
| ATOM | 8572 | OH2 | WAT | W | 390 | 39.193 | 43.075 | 22.527 | 1.00 | 29.86 | W |
| ATOM | 8573 | OH2 | WAT | W | 391 | 16.422 | 52.881 | −22.476 | 1.00 | 34.66 | W |
| ATOM | 8574 | OH2 | WAT | W | 392 | 27.247 | 35.033 | 29.275 | 1.00 | 28.11 | W |
| ATOM | 8575 | OH2 | WAT | W | 393 | 17.206 | 80.910 | −7.468 | 1.00 | 30.36 | W |
| ATOM | 8576 | OH2 | WAT | W | 394 | 48.207 | 41.926 | 3.615 | 1.00 | 28.31 | W |
| ATOM | 8577 | OH2 | WAT | W | 395 | 73.428 | 51.496 | −11.339 | 1.00 | 36.34 | W |
| ATOM | 8578 | OH2 | WAT | W | 396 | 58.697 | 53.866 | −22.198 | 1.00 | 22.68 | W |
| ATOM | 8579 | OH2 | WAT | W | 397 | 47.595 | 79.692 | −18.600 | 1.00 | 24.41 | W |
| ATOM | 8580 | OH2 | WAT | W | 398 | 40.846 | 96.688 | −37.242 | 1.00 | 26.13 | W |
| ATOM | 8581 | OH2 | WAT | W | 399 | 61.087 | 63.855 | 15.193 | 1.00 | 28.95 | W |
| ATOM | 8582 | OH2 | WAT | W | 400 | 59.255 | 37.753 | −15.381 | 1.00 | 33.77 | W |
| ATOM | 8583 | OH2 | WAT | W | 401 | 21.106 | 30.423 | 17.584 | 1.00 | 37.08 | W |
| ATOM | 8584 | OH2 | WAT | W | 402 | 46.149 | 97.302 | −43.888 | 1.00 | 30.85 | W |
| ATOM | 8585 | OH2 | WAT | W | 403 | 21.596 | 86.357 | −19.077 | 1.00 | 33.24 | W |
| ATOM | 8586 | OH2 | WAT | W | 404 | 20.559 | 68.440 | 22.185 | 1.00 | 27.53 | W |
| ATOM | 8587 | OH2 | WAT | W | 405 | 45.436 | 70.103 | −18.246 | 1.00 | 28.94 | W |
| ATOM | 8588 | OH2 | WAT | W | 406 | 17.493 | 70.844 | 6.619 | 1.00 | 35.50 | W |
| ATOM | 8589 | OH2 | WAT | W | 407 | 51.184 | 52.976 | −28.552 | 1.00 | 26.56 | W |
| ATOM | 8590 | OH2 | WAT | W | 408 | 19.132 | 83.539 | −25.709 | 1.00 | 32.11 | W |
| ATOM | 8591 | OH2 | WAT | W | 409 | 40.020 | 86.351 | −21.792 | 1.00 | 26.97 | W |
| ATOM | 8592 | OH2 | WAT | W | 410 | 22.565 | 66.347 | 29.045 | 1.00 | 35.45 | W |
| ATOM | 8593 | OH2 | WAT | W | 411 | 24.909 | 59.408 | −36.350 | 1.00 | 33.36 | W |
| ATOM | 8594 | OH2 | WAT | W | 412 | 58.513 | 34.945 | −5.934 | 1.00 | 31.42 | W |
| ATOM | 8595 | OH2 | WAT | W | 413 | 50.569 | 79.331 | −15.684 | 1.00 | 27.02 | W |
| ATOM | 8596 | OH2 | WAT | W | 414 | 17.529 | 68.704 | 24.307 | 1.00 | 44.71 | W |
| ATOM | 8597 | OH2 | WAT | W | 415 | 56.496 | 93.001 | −29.377 | 1.00 | 26.99 | W |
| ATOM | 8598 | OH2 | WAT | W | 416 | 52.554 | 73.396 | 4.320 | 1.00 | 35.30 | W |
| ATOM | 8599 | OH2 | WAT | W | 417 | 21.928 | 36.838 | 0.097 | 1.00 | 26.39 | W |
| ATOM | 8600 | OH2 | WAT | W | 418 | 40.066 | 79.962 | −28.061 | 1.00 | 30.33 | W |
| ATOM | 8601 | OH2 | WAT | W | 419 | 10.502 | 77.343 | −5.022 | 1.00 | 32.53 | W |
| ATOM | 8602 | OH2 | WAT | W | 420 | 26.186 | 64.608 | 8.450 | 1.00 | 14.74 | W |
| ATOM | 8603 | OH2 | WAT | W | 421 | 72.878 | 64.115 | −21.052 | 1.00 | 28.29 | W |
| ATOM | 8604 | OH2 | WAT | W | 422 | 79.844 | 74.115 | −17.720 | 1.00 | 34.35 | W |
| ATOM | 8605 | OH2 | WAT | W | 423 | 66.750 | 74.589 | −4.406 | 1.00 | 29.82 | W |
| ATOM | 8606 | OH2 | WAT | W | 424 | 48.124 | 64.413 | −28.378 | 1.00 | 28.57 | W |
| ATOM | 8607 | OH2 | WAT | W | 425 | 42.583 | 39.574 | −1.485 | 1.00 | 33.08 | W |
| ATOM | 8608 | OH2 | WAT | W | 426 | 63.043 | 67.969 | −31.856 | 1.00 | 31.53 | W |
| ATOM | 8609 | OH2 | WAT | W | 427 | 24.135 | 42.133 | −14.640 | 1.00 | 26.73 | W |
| ATOM | 8610 | OH2 | WAT | W | 428 | 27.597 | 50.981 | 13.326 | 1.00 | 22.07 | W |
| ATOM | 8611 | OH2 | WAT | W | 429 | 38.642 | 83.743 | −39.756 | 1.00 | 27.77 | W |
| ATOM | 8612 | OH2 | WAT | W | 430 | 76.638 | 74.336 | −26.811 | 1.00 | 31.44 | W |
| ATOM | 8613 | OH2 | WAT | W | 431 | 28.844 | 72.533 | 22.350 | 1.00 | 24.75 | W |
| ATOM | 8614 | OH2 | WAT | W | 432 | 20.733 | 55.289 | −35.819 | 1.00 | 32.46 | W |
| ATOM | 8615 | OH2 | WAT | W | 433 | 24.102 | 57.306 | 38.832 | 1.00 | 32.79 | W |
| ATOM | 8616 | OH2 | WAT | W | 434 | 38.103 | 49.334 | −10.508 | 1.00 | 52.88 | W |
| ATOM | 8617 | OH2 | WAT | W | 435 | 23.463 | 82.820 | −40.699 | 1.00 | 25.30 | W |
| ATOM | 8618 | OH2 | WAT | W | 436 | 17.159 | 67.178 | −26.028 | 1.00 | 32.43 | W |
| ATOM | 8619 | OH2 | WAT | W | 437 | 66.940 | 84.524 | −22.088 | 1.00 | 39.61 | W |
| ATOM | 8620 | OH2 | WAT | W | 438 | 52.706 | 65.072 | −28.075 | 1.00 | 53.96 | W |
| ATOM | 8621 | OH2 | WAT | W | 439 | 20.674 | 68.515 | 8.679 | 1.00 | 31.64 | W |
| ATOM | 8622 | OH2 | WAT | W | 440 | 61.511 | 64.004 | −30.897 | 1.00 | 35.45 | W |
| ATOM | 8623 | OH2 | WAT | W | 441 | 9.843 | 60.222 | 6.745 | 1.00 | 31.89 | W |
| ATOM | 8624 | OH2 | WAT | W | 442 | 31.554 | 86.043 | −19.875 | 1.00 | 28.33 | W |
| ATOM | 8625 | OH2 | WAT | W | 443 | 28.136 | 88.065 | −41.587 | 1.00 | 32.96 | W |
| ATOM | 8626 | OH2 | WAT | W | 444 | 44.810 | 82.831 | −30.419 | 1.00 | 22.80 | W |
| ATOM | 8627 | OH2 | WAT | W | 445 | 59.422 | 67.339 | −34.536 | 1.00 | 27.17 | W |
| ATOM | 8628 | OH2 | WAT | W | 446 | 70.306 | 56.764 | −12.670 | 1.00 | 30.28 | W |
| ATOM | 8629 | OH2 | WAT | W | 447 | 46.148 | 46.108 | 16.196 | 1.00 | 38.72 | W |
| ATOM | 8630 | OH2 | WAT | W | 448 | 46.024 | 83.007 | −25.271 | 1.00 | 30.80 | W |
| ATOM | 8631 | OH2 | WAT | W | 449 | 56.891 | 87.883 | −19.945 | 1.00 | 30.66 | W |
| ATOM | 8632 | OH2 | WAT | W | 450 | 73.703 | 74.266 | −5.840 | 1.00 | 24.67 | W |
| ATOM | 8633 | OH2 | WAT | W | 451 | 19.765 | 43.348 | 33.805 | 1.00 | 26.93 | W |
| ATOM | 8634 | OH2 | WAT | W | 452 | 79.355 | 51.266 | −0.720 | 1.00 | 22.05 | W |
| ATOM | 8635 | OH2 | WAT | W | 453 | 65.936 | 45.515 | −3.341 | 1.00 | 33.03 | W |
| ATOM | 8636 | OH2 | WAT | W | 454 | 48.270 | 78.072 | −9.661 | 1.00 | 26.82 | W |
| ATOM | 8637 | OH2 | WAT | W | 455 | 30.106 | 34.247 | −1.347 | 1.00 | 31.61 | W |
| ATOM | 8638 | OH2 | WAT | W | 456 | 16.244 | 75.167 | −24.092 | 1.00 | 37.53 | W |
| ATOM | 8639 | OH2 | WAT | W | 457 | 17.587 | 39.584 | 35.892 | 1.00 | 43.47 | W |
| ATOM | 8640 | OH2 | WAT | W | 458 | 22.585 | 31.274 | 29.286 | 1.00 | 34.35 | W |
| ATOM | 8641 | OH2 | WAT | W | 459 | 33.432 | 84.505 | −18.281 | 1.00 | 18.32 | W |
| ATOM | 8642 | OH2 | WAT | W | 460 | 41.293 | 95.068 | −39.456 | 1.00 | 30.13 | W |
| ATOM | 8643 | OH2 | WAT | W | 461 | 44.216 | 83.808 | −27.693 | 1.00 | 29.54 | W |
| ATOM | 8644 | OH2 | WAT | W | 462 | 30.848 | 57.443 | 4.499 | 1.00 | 24.35 | W |
| ATOM | 8645 | OH2 | WAT | W | 463 | 65.858 | 66.382 | −0.390 | 1.00 | 23.54 | W |
| ATOM | 8646 | OH2 | WAT | W | 464 | 46.496 | 68.686 | 26.430 | 1.00 | 34.50 | W |
| ATOM | 8647 | OH2 | WAT | W | 465 | 70.058 | 61.949 | −20.503 | 1.00 | 32.36 | W |
| ATOM | 8648 | OH2 | WAT | W | 466 | 23.603 | 91.171 | −25.989 | 1.00 | 36.74 | W |
| ATOM | 8649 | OH2 | WAT | W | 467 | 25.313 | 71.229 | 22.334 | 1.00 | 35.00 | W |
| ATOM | 8650 | OH2 | WAT | W | 468 | 13.930 | 69.551 | 26.567 | 1.00 | 25.16 | W |
| ATOM | 8651 | OH2 | WAT | W | 469 | 62.150 | 80.957 | −19.141 | 1.00 | 29.07 | W |
| ATOM | 8652 | OH2 | WAT | W | 470 | 27.702 | 47.846 | 45.920 | 1.00 | 36.32 | W |
| ATOM | 8653 | OH2 | WAT | W | 471 | 24.849 | 34.267 | 0.227 | 1.00 | 38.79 | W |
| ATOM | 8654 | OH2 | WAT | W | 472 | 57.544 | 69.087 | 13.346 | 1.00 | 41.68 | W |
| ATOM | 8655 | OH2 | WAT | W | 473 | 7.918 | 47.992 | 11.814 | 1.00 | 26.20 | W |
| ATOM | 8656 | OH2 | WAT | W | 474 | 71.374 | 59.946 | 0.845 | 1.00 | 32.73 | W |
| ATOM | 8657 | OH2 | WAT | W | 475 | 67.611 | 88.261 | −23.317 | 1.00 | 26.99 | W |
| ATOM | 8658 | OH2 | WAT | W | 476 | 12.265 | 75.299 | −13.600 | 1.00 | 38.45 | W |
| ATOM | 8659 | OH2 | WAT | W | 477 | 28.053 | 52.171 | 42.293 | 1.00 | 30.60 | W |
| ATOM | 8660 | OH2 | WAT | W | 478 | 22.099 | 62.997 | 33.586 | 1.00 | 29.07 | W |
| ATOM | 8661 | OH2 | WAT | W | 479 | 59.364 | 67.033 | −26.345 | 1.00 | 55.26 | W |
| ATOM | 8662 | OH2 | WAT | W | 480 | 34.109 | 87.415 | −10.585 | 1.00 | 34.81 | W |
| ATOM | 8663 | OH2 | WAT | W | 481 | 30.244 | 36.564 | 29.055 | 1.00 | 26.04 | W |
| ATOM | 8664 | OH2 | WAT | W | 482 | 8.622 | 49.766 | 5.623 | 1.00 | 39.99 | W |
| ATOM | 8665 | OH2 | WAT | W | 483 | 41.947 | 67.496 | 29.311 | 1.00 | 29.88 | W |
| ATOM | 8666 | OH2 | WAT | W | 484 | 72.522 | 75.947 | −27.187 | 1.00 | 34.08 | W |
| ATOM | 8667 | OH2 | WAT | W | 485 | 8.810 | 44.612 | 12.806 | 1.00 | 30.32 | W |
| ATOM | 8668 | OH2 | WAT | W | 486 | 71.081 | 43.226 | −15.424 | 1.00 | 37.75 | W |
| ATOM | 8669 | OH2 | WAT | W | 487 | 41.603 | 44.813 | −24.389 | 1.00 | 26.97 | W |
| ATOM | 8670 | OH2 | WAT | W | 488 | 39.942 | 66.979 | 31.389 | 1.00 | 33.16 | W |
| ATOM | 8671 | OH2 | WAT | W | 489 | 53.330 | 52.741 | 26.220 | 1.00 | 30.83 | W |
| ATOM | 8672 | OH2 | WAT | W | 490 | 26.012 | 44.653 | −26.984 | 1.00 | 30.08 | W |
| ATOM | 8673 | OH2 | WAT | W | 491 | 19.614 | 52.647 | −27.178 | 1.00 | 30.06 | W |
| ATOM | 8674 | OH2 | WAT | W | 492 | 71.069 | 95.514 | −34.992 | 1.00 | 35.85 | W |
| ATOM | 8675 | OH2 | WAT | W | 493 | 47.995 | 64.623 | −25.770 | 1.00 | 28.47 | W |
| ATOM | 8676 | OH2 | WAT | W | 494 | 44.647 | 64.565 | −35.193 | 1.00 | 43.57 | W |
| ATOM | 8677 | OH2 | WAT | W | 495 | 17.869 | 53.128 | 35.601 | 1.00 | 33.86 | W |
| ATOM | 8678 | OH2 | WAT | W | 496 | 83.555 | 70.250 | −20.339 | 1.00 | 26.44 | W |
| ATOM | 8679 | OH2 | WAT | W | 497 | 43.886 | 79.681 | −42.675 | 1.00 | 33.33 | W |
| ATOM | 8680 | OH2 | WAT | W | 498 | 22.649 | 62.755 | 16.163 | 1.00 | 30.46 | W |
| ATOM | 8681 | OH2 | WAT | W | 499 | 45.787 | 79.442 | −8.060 | 1.00 | 26.37 | W |
| ATOM | 8682 | OH2 | WAT | W | 500 | 26.612 | 88.201 | −4.086 | 1.00 | 36.66 | W |
| ATOM | 8683 | OH2 | WAT | W | 501 | 38.568 | 42.137 | −22.285 | 1.00 | 35.38 | W |
| ATOM | 8684 | OH2 | WAT | W | 502 | 36.970 | 29.325 | 12.903 | 1.00 | 40.61 | W |
| ATOM | 8685 | OH2 | WAT | W | 503 | 20.373 | 65.690 | 27.143 | 1.00 | 29.98 | W |
| ATOM | 8686 | OH2 | WAT | W | 504 | 6.788 | 59.476 | 2.793 | 1.00 | 39.82 | W |
| ATOM | 8687 | OH2 | WAT | W | 505 | 61.162 | 77.612 | −12.985 | 1.00 | 26.24 | W |
| ATOM | 8688 | OH2 | WAT | W | 506 | 28.083 | 49.302 | 38.517 | 1.00 | 29.75 | W |
| ATOM | 8689 | OH2 | WAT | W | 507 | 29.143 | 41.147 | −25.689 | 1.00 | 30.45 | W |
| ATOM | 8690 | OH2 | WAT | W | 508 | 19.441 | 67.572 | −2.825 | 1.00 | 31.10 | W |
| ATOM | 8691 | OH2 | WAT | W | 509 | 43.421 | 41.867 | −7.636 | 1.00 | 25.84 | W |
| ATOM | 8692 | OH2 | WAT | W | 510 | 48.778 | 55.138 | −22.650 | 1.00 | 24.48 | W |
| ATOM | 8693 | OH2 | WAT | W | 511 | 26.304 | 81.967 | −3.669 | 1.00 | 59.92 | W |
| ATOM | 8694 | OH2 | WAT | W | 512 | 35.823 | 38.962 | −16.238 | 1.00 | 34.57 | W |
| ATOM | 8695 | OH2 | WAT | W | 513 | 58.609 | 51.303 | 22.182 | 1.00 | 21.16 | W |
| ATOM | 8696 | OH2 | WAT | W | 514 | 53.978 | 45.768 | 4.798 | 1.00 | 38.64 | W |
| ATOM | 8697 | OH2 | WAT | W | 515 | 46.492 | 80.496 | −42.683 | 1.00 | 36.59 | W |
| ATOM | 8698 | OH2 | WAT | W | 516 | 71.760 | 84.670 | −19.901 | 1.00 | 26.72 | W |
| ATOM | 8699 | OH2 | WAT | W | 517 | 61.084 | 67.579 | −36.593 | 1.00 | 31.75 | W |
| ATOM | 8700 | OH2 | WAT | W | 518 | 38.336 | 31.513 | 16.723 | 1.00 | 46.16 | W |
| ATOM | 8701 | OH2 | WAT | W | 519 | 58.345 | 34.917 | −12.173 | 1.00 | 32.99 | W |
| ATOM | 8702 | OH2 | WAT | W | 520 | 21.043 | 79.711 | −23.638 | 1.00 | 26.06 | W |
| ATOM | 8703 | OH2 | WAT | W | 521 | 51.272 | 71.528 | −20.158 | 1.00 | 20.05 | W |
| ATOM | 8704 | OH2 | WAT | W | 522 | 32.016 | 85.400 | −8.674 | 1.00 | 29.42 | W |
| ATOM | 8705 | OH2 | WAT | W | 523 | 21.535 | 29.268 | 27.784 | 1.00 | 34.66 | W |
| ATOM | 8706 | OH2 | WAT | W | 524 | 21.912 | 87.705 | −6.947 | 1.00 | 46.50 | W |
| ATOM | 8707 | OH2 | WAT | W | 525 | 31.620 | 57.576 | 37.407 | 1.00 | 24.48 | W |
| ATOM | 8708 | OH2 | WAT | W | 526 | 17.389 | 81.606 | −17.144 | 1.00 | 43.70 | W |
| ATOM | 8709 | OH2 | WAT | W | 527 | 51.987 | 59.263 | 6.165 | 1.00 | 56.07 | W |
| ATOM | 8710 | OH2 | WAT | W | 528 | 22.039 | 80.414 | −35.965 | 1.00 | 28.87 | W |
| ATOM | 8711 | OH2 | WAT | W | 529 | 25.523 | 74.218 | 8.623 | 1.00 | 19.22 | W |
| ATOM | 8712 | OH2 | WAT | W | 530 | 21.682 | 80.087 | 5.339 | 1.00 | 34.53 | W |
| ATOM | 8713 | OH2 | WAT | W | 531 | 48.616 | 94.265 | −37.235 | 1.00 | 43.59 | W |
| ATOM | 8714 | OH2 | WAT | W | 532 | 39.857 | 38.874 | −3.549 | 1.00 | 31.28 | W |
| ATOM | 8715 | OH2 | WAT | W | 533 | 39.693 | 96.380 | −28.089 | 1.00 | 44.71 | W |
| ATOM | 8716 | OH2 | WAT | W | 534 | 36.699 | 35.085 | −15.806 | 1.00 | 33.87 | W |
| ATOM | 8717 | OH2 | WAT | W | 535 | 34.981 | 45.880 | 33.336 | 1.00 | 17.58 | W |
| ATOM | 8718 | OH2 | WAT | W | 536 | 55.752 | 78.071 | −1.645 | 1.00 | 36.67 | W |
| ATOM | 8719 | OH2 | WAT | W | 537 | 69.964 | 62.216 | −0.087 | 1.00 | 33.47 | W |
| ATOM | 8720 | OH2 | WAT | W | 538 | 46.113 | 74.500 | 22.679 | 1.00 | 31.71 | W |
| ATOM | 8721 | OH2 | WAT | W | 539 | 48.482 | 46.071 | −23.200 | 1.00 | 33.97 | W |
| ATOM | 8722 | OH2 | WAT | W | 540 | 60.421 | 92.222 | −28.586 | 1.00 | 34.39 | W |
| ATOM | 8723 | OH2 | WAT | W | 541 | 37.644 | 62.372 | 35.379 | 1.00 | 36.29 | W |
| ATOM | 8724 | OH2 | WAT | W | 542 | 29.209 | 63.265 | −37.460 | 1.00 | 56.13 | W |
| ATOM | 8725 | OH2 | WAT | W | 543 | 61.264 | 48.115 | −31.707 | 1.00 | 49.40 | W |
| ATOM | 8726 | OH2 | WAT | W | 544 | 61.974 | 83.566 | −38.096 | 1.00 | 31.08 | W |
| ATOM | 8727 | OH2 | WAT | W | 545 | 62.940 | 42.943 | −20.920 | 1.00 | 27.15 | W |
| ATOM | 8728 | OH2 | WAT | W | 546 | 53.465 | 44.416 | 22.714 | 1.00 | 34.54 | W |
| ATOM | 8729 | OH2 | WAT | W | 547 | 30.815 | 34.793 | −9.434 | 1.00 | 36.45 | W |
| ATOM | 8730 | OH2 | WAT | W | 548 | 29.471 | 31.641 | 21.751 | 1.00 | 34.56 | W |
| ATOM | 8731 | OH2 | WAT | W | 549 | 78.607 | 69.017 | −12.205 | 1.00 | 28.49 | W |
| ATOM | 8732 | OH2 | WAT | W | 550 | 31.429 | 69.250 | −45.559 | 1.00 | 34.20 | W |
| ATOM | 8733 | OH2 | WAT | W | 551 | 73.435 | 82.540 | −21.336 | 1.00 | 27.02 | W |
| ATOM | 8734 | OH2 | WAT | W | 552 | 47.327 | 71.323 | −23.894 | 1.00 | 41.40 | W |
| ATOM | 8735 | OH2 | WAT | W | 553 | 20.476 | 37.997 | 12.047 | 1.00 | 29.83 | W |
| ATOM | 8736 | OH2 | WAT | W | 554 | 35.058 | 95.246 | −32.227 | 1.00 | 34.09 | W |
| ATOM | 8737 | OH2 | WAT | W | 555 | 17.108 | 41.547 | 9.120 | 1.00 | 36.08 | W |
| ATOM | 8738 | OH2 | WAT | W | 556 | 20.640 | 80.355 | −33.668 | 1.00 | 40.07 | W |
| ATOM | 8739 | OH2 | WAT | W | 557 | 49.088 | 92.026 | −24.040 | 1.00 | 33.71 | W |
| ATOM | 8740 | OH2 | WAT | W | 558 | 43.763 | 44.871 | 19.288 | 1.00 | 34.59 | W |
| ATOM | 8741 | OH2 | WAT | W | 559 | 21.204 | 35.660 | −5.134 | 1.00 | 32.08 | W |
| ATOM | 8742 | OH2 | WAT | W | 560 | 65.009 | 88.087 | −24.210 | 1.00 | 29.80 | W |
| ATOM | 8743 | OH2 | WAT | W | 561 | 46.916 | 53.996 | −21.422 | 1.00 | 33.60 | W |
| ATOM | 8744 | OH2 | WAT | W | 562 | 71.404 | 63.255 | −27.114 | 1.00 | 40.61 | W |
| ATOM | 8745 | OH2 | WAT | W | 563 | 20.015 | 35.616 | 25.763 | 1.00 | 30.97 | W |
| ATOM | 8746 | OH2 | WAT | W | 564 | 59.355 | 87.240 | −45.689 | 1.00 | 37.04 | W |
| ATOM | 8747 | OH2 | WAT | W | 565 | 51.864 | 34.258 | −7.762 | 1.00 | 49.22 | W |
| ATOM | 8748 | OH2 | WAT | W | 566 | 56.208 | 79.437 | −15.027 | 1.00 | 37.82 | W |
| ATOM | 8749 | OH2 | WAT | W | 567 | 28.902 | 70.034 | −39.462 | 1.00 | 28.91 | W |
| ATOM | 8750 | OH2 | WAT | W | 568 | 45.154 | 83.397 | −43.261 | 1.00 | 42.35 | W |
| ATOM | 8751 | OH2 | WAT | W | 569 | 29.727 | 81.729 | −42.805 | 1.00 | 41.68 | W |
| ATOM | 8752 | OH2 | WAT | W | 570 | 28.533 | 90.869 | −42.604 | 1.00 | 31.64 | W |
| ATOM | 8753 | OH2 | WAT | W | 571 | 19.677 | 84.860 | −28.486 | 1.00 | 44.44 | W |
| ATOM | 8754 | OH2 | WAT | W | 572 | 37.852 | 97.435 | −30.634 | 1.00 | 44.72 | W |
| ATOM | 8755 | OH2 | WAT | W | 573 | 40.615 | 44.612 | −31.597 | 1.00 | 42.55 | W |
| ATOM | 8756 | OH2 | WAT | W | 574 | 21.022 | 41.654 | 35.102 | 1.00 | 36.46 | W |
| ATOM | 8757 | OH2 | WAT | W | 575 | 39.353 | 30.564 | 13.190 | 1.00 | 48.83 | W |
| ATOM | 8758 | OH2 | WAT | W | 576 | 62.189 | 85.929 | −23.040 | 1.00 | 32.28 | W |
| ATOM | 8759 | OH2 | WAT | W | 577 | 31.076 | 68.489 | 14.056 | 1.00 | 15.60 | W |
| ATOM | 8760 | OH2 | WAT | W | 578 | 28.012 | 67.450 | 33.693 | 1.00 | 40.99 | W |
| ATOM | 8761 | OH2 | WAT | W | 579 | 14.920 | 63.590 | −25.823 | 1.00 | 25.17 | W |
| ATOM | 8762 | OH2 | WAT | W | 580 | 29.779 | 72.346 | 12.292 | 1.00 | 14.98 | W |
| ATOM | 8763 | OH2 | WAT | W | 581 | 61.161 | 93.203 | −38.580 | 1.00 | 45.25 | W |
| ATOM | 8764 | OH2 | WAT | W | 582 | 59.322 | 62.288 | 25.727 | 1.00 | 29.38 | W |
| ATOM | 8765 | OH2 | WAT | W | 583 | 51.208 | 79.524 | −1.693 | 1.00 | 29.35 | W |
| ATOM | 8766 | OH2 | WAT | W | 584 | 21.375 | 60.546 | 26.774 | 1.00 | 52.30 | W |
| ATOM | 8767 | OH2 | WAT | W | 585 | 59.422 | 91.535 | −35.103 | 1.00 | 27.02 | W |
| ATOM | 8768 | OH2 | WAT | W | 586 | 24.246 | 60.834 | 35.994 | 1.00 | 32.50 | W |
| ATOM | 8769 | OH2 | WAT | W | 587 | 11.430 | 62.017 | 0.147 | 1.00 | 40.24 | W |
| ATOM | 8770 | OH2 | WAT | W | 588 | 55.140 | 80.683 | −19.320 | 1.00 | 24.62 | W |
| ATOM | 8771 | OH2 | WAT | W | 589 | 16.965 | 74.567 | −28.649 | 1.00 | 38.99 | W |
| ATOM | 8772 | OH2 | WAT | W | 590 | 40.708 | 40.703 | −21.403 | 1.00 | 30.96 | W |
| ATOM | 8773 | OH2 | WAT | W | 591 | 28.184 | 68.576 | −46.105 | 1.00 | 47.34 | W |
| ATOM | 8774 | OH2 | WAT | W | 592 | 40.001 | 35.508 | 11.320 | 1.00 | 23.27 | W |
| ATOM | 8775 | OH2 | WAT | W | 593 | 19.051 | 80.665 | −11.861 | 1.00 | 31.12 | W |
| ATOM | 8776 | OH2 | WAT | W | 594 | 64.968 | 80.063 | −8.839 | 1.00 | 41.14 | W |
| ATOM | 8777 | OH2 | WAT | W | 595 | 72.984 | 43.604 | −12.198 | 1.00 | 34.73 | W |
| ATOM | 8778 | OH2 | WAT | W | 596 | 12.889 | 74.876 | −19.207 | 1.00 | 37.16 | W |
| ATOM | 8779 | OH2 | WAT | W | 597 | 57.083 | 75.724 | −2.830 | 1.00 | 31.22 | W |
| ATOM | 8780 | OH2 | WAT | W | 598 | 20.946 | 58.561 | 37.844 | 1.00 | 38.60 | W |
| ATOM | 8781 | OH2 | WAT | W | 599 | 50.726 | 77.525 | 2.133 | 1.00 | 40.85 | W |
| ATOM | 8782 | OH2 | WAT | W | 600 | 28.394 | 45.572 | 40.705 | 1.00 | 44.08 | W |
| ATOM | 8783 | OH2 | WAT | W | 601 | 23.052 | 36.982 | 13.354 | 1.00 | 27.57 | W |
| ATOM | 8784 | OH2 | WAT | W | 602 | 33.276 | 39.668 | −19.126 | 1.00 | 45.50 | W |
| ATOM | 8785 | OH2 | WAT | W | 603 | 33.717 | 49.409 | −15.061 | 1.00 | 49.24 | W |
| ATOM | 8786 | OH2 | WAT | W | 604 | 29.452 | 88.986 | −26.113 | 1.00 | 14.64 | W |
| ATOM | 8787 | OH2 | WAT | W | 605 | 37.094 | 33.825 | 5.788 | 1.00 | 36.96 | W |
| ATOM | 8788 | OH2 | WAT | W | 606 | 71.840 | 62.869 | −18.101 | 1.00 | 33.45 | W |
| ATOM | 8789 | OH2 | WAT | W | 607 | 34.316 | 70.286 | −44.856 | 1.00 | 35.57 | W |
| ATOM | 8790 | OH2 | WAT | W | 608 | 64.261 | 77.372 | 0.343 | 1.00 | 41.73 | W |
| ATOM | 8791 | OH2 | WAT | W | 609 | 35.766 | 55.966 | 8.537 | 1.00 | 61.84 | W |
| ATOM | 8792 | OH2 | WAT | W | 610 | 30.712 | 51.359 | 39.606 | 1.00 | 41.26 | W |
| ATOM | 8793 | OH2 | WAT | W | 611 | 54.267 | 58.059 | −13.818 | 1.00 | 54.40 | W |
| ATOM | 8794 | OH2 | WAT | W | 612 | 21.028 | 54.609 | 46.901 | 1.00 | 38.62 | W |
| ATOM | 8795 | OH2 | WAT | W | 613 | 14.508 | 50.708 | 0.979 | 1.00 | 32.56 | W |
| ATOM | 8796 | OH2 | WAT | W | 614 | 25.322 | 91.719 | −32.996 | 1.00 | 42.32 | W |
| ATOM | 8797 | OH2 | WAT | W | 615 | 73.153 | 71.399 | −29.195 | 1.00 | 42.43 | W |
| ATOM | 8798 | OH2 | WAT | W | 616 | 55.707 | 38.604 | 0.984 | 1.00 | 44.24 | W |
| ATOM | 8799 | OH2 | WAT | W | 617 | 37.463 | 84.235 | 4.510 | 1.00 | 42.83 | W |
| ATOM | 8800 | OH2 | WAT | W | 618 | 45.682 | 55.261 | 10.267 | 1.00 | 45.85 | W |
| ATOM | 8801 | OH2 | WAT | W | 619 | 67.043 | 81.358 | −11.165 | 1.00 | 31.78 | W |
| ATOM | 8802 | OH2 | WAT | W | 620 | 40.544 | 89.334 | −19.559 | 1.00 | 47.91 | W |
| ATOM | 8803 | OH2 | WAT | W | 621 | 43.777 | 56.722 | 33.517 | 1.00 | 27.63 | W |
| ATOM | 8804 | OH2 | WAT | W | 622 | 56.281 | 50.279 | −30.462 | 1.00 | 48.83 | W |
| ATOM | 8805 | OH2 | WAT | W | 623 | 63.571 | 57.555 | 26.026 | 1.00 | 41.71 | W |
| ATOM | 8806 | OH2 | WAT | W | 624 | 62.158 | 48.421 | −20.935 | 1.00 | 38.60 | W |
| ATOM | 8807 | OH2 | WAT | W | 625 | 62.877 | 59.693 | −22.146 | 1.00 | 60.99 | W |
| ATOM | 8808 | OH2 | WAT | W | 626 | 39.812 | 77.874 | 13.633 | 1.00 | 45.54 | W |
| ATOM | 8809 | OH2 | WAT | W | 627 | 26.711 | 91.998 | −27.596 | 1.00 | 30.68 | W |
| ATOM | 8810 | OH2 | WAT | W | 628 | 13.505 | 70.317 | −9.667 | 1.00 | 32.85 | W |
| ATOM | 8811 | OH2 | WAT | W | 629 | 60.642 | 79.086 | −41.474 | 1.00 | 27.69 | W |
| ATOM | 8812 | OH2 | WAT | W | 630 | 39.181 | 43.469 | 26.620 | 1.00 | 39.36 | W |
| ATOM | 8813 | OH2 | WAT | W | 631 | 11.991 | 71.039 | 2.437 | 1.00 | 33.17 | W |
| ATOM | 8814 | OH2 | WAT | W | 632 | 36.447 | 62.759 | −38.362 | 1.00 | 30.08 | W |
| ATOM | 8815 | OH2 | WAT | W | 633 | 75.969 | 83.252 | −25.278 | 1.00 | 45.04 | W |
| ATOM | 8816 | OH2 | WAT | W | 634 | 37.698 | 80.761 | −0.575 | 1.00 | 24.24 | W |
| ATOM | 8817 | OH2 | WAT | W | 635 | 47.369 | 53.715 | 35.730 | 1.00 | 45.08 | W |
| ATOM | 8818 | OH2 | WAT | W | 636 | 48.732 | 98.012 | −33.807 | 1.00 | 34.63 | W |
| ATOM | 8819 | OH2 | WAT | W | 637 | 63.265 | 75.050 | −45.725 | 1.00 | 38.33 | W |
| ATOM | 8820 | OH2 | WAT | W | 638 | 60.032 | 71.956 | 0.252 | 1.00 | 44.76 | W |
| ATOM | 8821 | OH2 | WAT | W | 639 | 40.988 | 48.143 | −29.350 | 1.00 | 45.67 | W |
| ATOM | 8822 | OH2 | WAT | W | 640 | 55.258 | 40.282 | −12.634 | 1.00 | 60.28 | W |
| ATOM | 8823 | OH2 | WAT | W | 641 | 75.283 | 84.086 | −22.829 | 1.00 | 44.58 | W |
| ATOM | 8824 | OH2 | WAT | W | 642 | 63.431 | 52.143 | −0.405 | 1.00 | 29.19 | W |
| ATOM | 8825 | OH2 | WAT | W | 643 | 37.172 | 36.738 | 19.246 | 1.00 | 53.77 | W |
| ATOM | 8826 | OH2 | WAT | W | 644 | 57.876 | 36.061 | −2.371 | 1.00 | 48.71 | W |
| ATOM | 8827 | OH2 | WAT | W | 645 | 23.212 | 48.887 | −36.113 | 1.00 | 40.06 | W |
| ATOM | 8828 | OH2 | WAT | W | 646 | 18.060 | 71.467 | −29.825 | 1.00 | 43.44 | W |
| ATOM | 8829 | OH2 | WAT | W | 647 | 30.795 | 40.421 | 38.172 | 1.00 | 47.17 | W |
| ATOM | 8830 | OH2 | WAT | W | 648 | 27.612 | 50.327 | 23.213 | 1.00 | 31.47 | W |
| ATOM | 8831 | OH2 | WAT | W | 649 | 30.574 | 47.809 | 40.324 | 1.00 | 45.57 | W |
| ATOM | 8832 | OH2 | WAT | W | 650 | 59.939 | 52.951 | −30.672 | 1.00 | 35.35 | W |
| ATOM | 8833 | OH2 | WAT | W | 651 | 44.795 | 40.676 | 13.160 | 1.00 | 33.50 | W |
| ATOM | 8834 | OH2 | WAT | W | 652 | 34.039 | 47.923 | 36.038 | 1.00 | 38.29 | W |
| ATOM | 8835 | OH2 | WAT | W | 653 | 27.160 | 82.334 | 3.179 | 1.00 | 31.70 | W |
| ATOM | 8836 | OH2 | WAT | W | 654 | 58.512 | 92.375 | −42.108 | 1.00 | 40.35 | W |
| ATOM | 8837 | OH2 | WAT | W | 655 | 49.129 | 52.314 | −37.841 | 1.00 | 44.78 | W |
| ATOM | 8838 | OH2 | WAT | W | 656 | 53.318 | 71.106 | −16.915 | 1.00 | 25.96 | W |
| ATOM | 8839 | OH2 | WAT | W | 657 | 57.576 | 62.821 | −15.538 | 1.00 | 50.70 | W |
| ATOM | 8840 | OH2 | WAT | W | 658 | 28.417 | 44.432 | −29.609 | 1.00 | 36.13 | W |
| ATOM | 8841 | OH2 | WAT | W | 659 | 49.113 | 49.428 | −38.284 | 1.00 | 45.00 | W |
| ATOM | 8842 | OH2 | WAT | W | 660 | 35.586 | 77.729 | −43.101 | 1.00 | 33.39 | W |
| ATOM | 8843 | OH2 | WAT | W | 661 | 9.841 | 58.788 | 17.855 | 1.00 | 42.14 | W |
| ATOM | 8844 | OH2 | WAT | W | 662 | 61.026 | 45.578 | −3.773 | 1.00 | 34.99 | W |
| ATOM | 8845 | OH2 | WAT | W | 663 | 65.493 | 78.967 | −5.769 | 1.00 | 29.19 | W |
| ATOM | 8846 | OH2 | WAT | W | 664 | 7.025 | 58.334 | 14.662 | 1.00 | 44.10 | W |
| ATOM | 8847 | OH2 | WAT | W | 665 | 49.654 | 50.855 | 32.514 | 1.00 | 30.08 | W |
| ATOM | 8848 | OH2 | WAT | W | 666 | 18.545 | 62.489 | 22.933 | 1.00 | 60.50 | W |
| ATOM | 8849 | OH2 | WAT | W | 667 | 30.379 | 34.204 | 21.118 | 1.00 | 44.65 | W |
| ATOM | 8850 | OH2 | WAT | W | 668 | 17.488 | 45.677 | −14.303 | 1.00 | 32.55 | W |
| ATOM | 8851 | OH2 | WAT | W | 669 | 28.754 | 55.348 | 39.968 | 1.00 | 33.20 | W |
| ATOM | 8852 | OH2 | WAT | W | 670 | 50.808 | 60.080 | −8.922 | 1.00 | 11.10 | W |
| ATOM | 8853 | OH2 | WAT | W | 671 | 43.864 | 63.224 | −28.667 | 1.00 | 10.75 | W |
| ATOM | 8854 | OH2 | WAT | W | 672 | 38.132 | 82.008 | −31.368 | 1.00 | 15.05 | W |
| ATOM | 8855 | OH2 | WAT | W | 673 | 32.332 | 39.227 | −4.953 | 1.00 | 11.43 | W |
| ATOM | 8856 | OH2 | WAT | W | 674 | 60.350 | 61.096 | −18.141 | 1.00 | 13.21 | W |
| ATOM | 8857 | OH2 | WAT | W | 675 | 63.171 | 58.710 | −1.441 | 1.00 | 12.30 | W |
| ATOM | 8858 | OH2 | WAT | W | 676 | 60.719 | 61.106 | −3.556 | 1.00 | 11.61 | W |
| ATOM | 8859 | OH2 | WAT | W | 677 | 46.645 | 74.659 | 7.750 | 1.00 | 13.33 | W |
| ATOM | 8860 | OH2 | WAT | W | 678 | 54.976 | 57.679 | −0.489 | 1.00 | 13.83 | W |
| ATOM | 8861 | OH2 | WAT | W | 679 | 30.171 | 75.105 | −29.268 | 1.00 | 12.68 | W |
| ATOM | 8862 | OH2 | WAT | W | 680 | 58.910 | 56.848 | −1.463 | 1.00 | 12.78 | W |
| ATOM | 8863 | OH2 | WAT | W | 681 | 58.707 | 52.780 | −0.068 | 1.00 | 16.81 | W |
| ATOM | 8864 | OH2 | WAT | W | 682 | 63.035 | 61.198 | −4.943 | 1.00 | 15.69 | W |
| ATOM | 8865 | OH2 | WAT | W | 683 | 27.871 | 48.752 | 12.133 | 1.00 | 14.85 | W |
| ATOM | 8866 | OH2 | WAT | W | 684 | 66.593 | 56.371 | −20.257 | 1.00 | 17.09 | W |
| ATOM | 8867 | OH2 | WAT | W | 685 | 19.823 | 46.911 | −14.372 | 1.00 | 18.34 | W |
| ATOM | 8868 | OH2 | WAT | W | 686 | 38.651 | 69.400 | −38.144 | 1.00 | 18.58 | W |
| ATOM | 8869 | OH2 | WAT | W | 687 | 33.756 | 60.443 | 23.244 | 1.00 | 16.84 | W |
| ATOM | 8870 | OH2 | WAT | W | 688 | 39.615 | 55.787 | 3.023 | 1.00 | 13.33 | W |
| ATOM | 8871 | OH2 | WAT | W | 689 | 24.685 | 61.650 | 9.133 | 1.00 | 15.12 | W |
| ATOM | 8872 | OH2 | WAT | W | 690 | 16.852 | 57.351 | 13.005 | 1.00 | 17.76 | W |
| ATOM | 8873 | OH2 | WAT | W | 691 | 39.357 | 69.360 | −35.452 | 1.00 | 16.14 | W |
| ATOM | 8874 | OH2 | WAT | W | 692 | 49.896 | 67.829 | 4.102 | 1.00 | 22.12 | W |
| ATOM | 8875 | OH2 | WAT | W | 693 | 27.767 | 53.520 | 20.006 | 1.00 | 16.13 | W |
| ATOM | 8876 | OH2 | WAT | W | 694 | 29.589 | 83.211 | 2.374 | 1.00 | 17.47 | W |
| ATOM | 8877 | OH2 | WAT | W | 695 | 28.136 | 91.131 | −25.283 | 1.00 | 18.51 | W |
| ATOM | 8878 | OH2 | WAT | W | 696 | 46.056 | 77.858 | 9.469 | 1.00 | 18.58 | W |
| ATOM | 8879 | OH2 | WAT | W | 697 | 12.598 | 49.461 | 17.773 | 1.00 | 23.12 | W |
| ATOM | 8880 | OH2 | WAT | W | 698 | 81.171 | 68.689 | −11.423 | 1.00 | 23.28 | W |
| ATOM | 8881 | OH2 | WAT | W | 699 | 41.447 | 44.917 | −6.984 | 1.00 | 18.12 | W |
| ATOM | 8882 | OH2 | WAT | W | 700 | 45.659 | 67.938 | −27.584 | 1.00 | 23.36 | W |
| ATOM | 8883 | OH2 | WAT | W | 701 | 14.273 | 50.690 | 24.204 | 1.00 | 20.19 | W |
| ATOM | 8884 | OH2 | WAT | W | 702 | 67.431 | 46.612 | −1.390 | 1.00 | 27.25 | W |
| ATOM | 8885 | OH2 | WAT | W | 703 | 9.075 | 50.498 | 8.166 | 1.00 | 25.31 | W |
| ATOM | 8886 | OH2 | WAT | W | 704 | 48.417 | 40.958 | −1.139 | 1.00 | 23.15 | W |
| ATOM | 8887 | OH2 | WAT | W | 705 | 17.999 | 74.642 | 3.920 | 1.00 | 32.70 | W |
| ATOM | 8888 | OH2 | WAT | W | 706 | 44.829 | 55.254 | −18.432 | 1.00 | 22.53 | W |
| ATOM | 8889 | OH2 | WAT | W | 707 | 83.508 | 66.537 | −15.459 | 1.00 | 23.10 | W |
| ATOM | 8890 | OH2 | WAT | W | 708 | 48.839 | 69.212 | −36.856 | 1.00 | 22.09 | W |
| ATOM | 8891 | OH2 | WAT | W | 709 | 51.752 | 70.380 | −38.057 | 1.00 | 23.90 | W |
| ATOM | 8892 | OH2 | WAT | W | 710 | 29.658 | 72.517 | 34.348 | 1.00 | 30.12 | W |
| ATOM | 8893 | OH2 | WAT | W | 711 | 37.525 | 61.580 | −32.546 | 1.00 | 23.53 | W |
| ATOM | 8894 | OH2 | WAT | W | 712 | 57.377 | 49.104 | 21.315 | 1.00 | 21.68 | W |
| ATOM | 8895 | OH2 | WAT | W | 713 | 42.963 | 59.616 | −31.428 | 1.00 | 23.43 | W |
| ATOM | 8896 | OH2 | WAT | W | 714 | 31.988 | 34.471 | −3.304 | 1.00 | 25.07 | W |
| ATOM | 8897 | OH2 | WAT | W | 715 | 37.084 | 59.237 | −33.588 | 1.00 | 24.72 | W |
| ATOM | 8898 | OH2 | WAT | W | 716 | 61.591 | 59.246 | 14.600 | 1.00 | 28.80 | W |
| ATOM | 8899 | OH2 | WAT | W | 717 | 40.198 | 56.424 | −19.000 | 1.00 | 23.02 | W |
| ATOM | 8900 | OH2 | WAT | W | 718 | 47.473 | 87.662 | −44.201 | 1.00 | 50.29 | W |
| ATOM | 8901 | OH2 | WAT | W | 719 | 61.734 | 76.696 | −10.423 | 1.00 | 16.92 | W |
| ATOM | 8902 | OH2 | WAT | W | 720 | 79.475 | 50.923 | −11.448 | 1.00 | 40.12 | W |
| ATOM | 8903 | OH2 | WAT | W | 721 | 37.556 | 51.763 | −18.478 | 1.00 | 21.63 | W |
| ATOM | 8904 | OH2 | WAT | W | 722 | 25.699 | 52.296 | 13.033 | 1.00 | 19.81 | W |
| ATOM | 8905 | OH2 | WAT | W | 723 | 69.562 | 64.077 | −28.574 | 1.00 | 28.25 | W |
| ATOM | 8906 | OH2 | WAT | W | 724 | 72.923 | 66.789 | −10.029 | 1.00 | 26.74 | W |
| ATOM | 8907 | OH2 | WAT | W | 725 | 42.712 | 56.570 | 2.479 | 1.00 | 32.14 | W |
| ATOM | 8908 | OH2 | WAT | W | 726 | 56.019 | 65.333 | −36.093 | 1.00 | 21.69 | W |
| ATOM | 8909 | OH2 | WAT | W | 727 | 17.870 | 66.389 | 28.045 | 1.00 | 31.05 | W |
| ATOM | 8910 | OH2 | WAT | W | 728 | 11.744 | 63.777 | −16.973 | 1.00 | 25.14 | W |
| ATOM | 8911 | OH2 | WAT | W | 729 | 35.294 | 86.458 | −17.918 | 1.00 | 29.79 | W |
| ATOM | 8912 | OH2 | WAT | W | 730 | 9.739 | 60.417 | −20.395 | 1.00 | 28.25 | W |
| ATOM | 8913 | OH2 | WAT | W | 731 | 11.654 | 48.520 | −2.460 | 1.00 | 33.38 | W |
| ATOM | 8914 | OH2 | WAT | W | 732 | 40.614 | 57.118 | 31.023 | 1.00 | 26.07 | W |
| ATOM | 8915 | OH2 | WAT | W | 733 | 39.777 | 53.158 | 36.413 | 1.00 | 33.49 | W |
| ATOM | 8916 | OH2 | WAT | W | 734 | 49.845 | 47.366 | 28.829 | 1.00 | 26.70 | W |
| ATOM | 8917 | OH2 | WAT | W | 735 | 24.110 | 34.457 | 15.334 | 1.00 | 29.67 | W |
| ATOM | 8918 | OH2 | WAT | W | 736 | 59.490 | 51.073 | 24.831 | 1.00 | 29.86 | W |
| ATOM | 8919 | OH2 | WAT | W | 737 | 38.054 | 83.336 | −1.120 | 1.00 | 28.89 | W |
| ATOM | 8920 | OH2 | WAT | W | 738 | 13.039 | 51.650 | −12.216 | 1.00 | 30.21 | W |
| ATOM | 8921 | OH2 | WAT | W | 739 | 48.500 | 50.823 | 35.082 | 1.00 | 34.12 | W |
| ATOM | 8922 | OH2 | WAT | W | 740 | 47.989 | 41.707 | −5.554 | 1.00 | 31.61 | W |
| ATOM | 8923 | OH2 | WAT | W | 741 | 22.205 | 40.959 | −11.330 | 1.00 | 33.16 | W |
| ATOM | 8924 | OH2 | WAT | W | 742 | 16.134 | 36.485 | 16.269 | 1.00 | 29.31 | W |
| ATOM | 8925 | OH2 | WAT | W | 743 | 22.480 | 68.170 | 20.286 | 1.00 | 24.77 | W |
| ATOM | 8926 | OH2 | WAT | W | 744 | 72.049 | 47.322 | −17.960 | 1.00 | 29.07 | W |
| ATOM | 8927 | OH2 | WAT | W | 745 | 40.857 | 85.387 | −11.973 | 1.00 | 23.51 | W |
| ATOM | 8928 | OH2 | WAT | W | 746 | 56.744 | 46.789 | 15.703 | 1.00 | 28.26 | W |
| ATOM | 8929 | OH2 | WAT | W | 747 | 51.904 | 64.105 | 24.543 | 1.00 | 27.18 | W |
| ATOM | 8930 | OH2 | WAT | W | 748 | 56.575 | 58.583 | 1.344 | 1.00 | 31.09 | W |
| ATOM | 8931 | OH2 | WAT | W | 749 | 57.373 | 58.561 | 5.484 | 1.00 | 29.43 | W |
| ATOM | 8932 | OH2 | WAT | W | 750 | 75.104 | 64.410 | −16.417 | 1.00 | 31.68 | W |
| ATOM | 8933 | OH2 | WAT | W | 751 | 14.670 | 70.784 | −23.138 | 1.00 | 25.91 | W |
| ATOM | 8934 | OH2 | WAT | W | 752 | 12.911 | 52.355 | −15.033 | 1.00 | 28.96 | W |
| ATOM | 8935 | OH2 | WAT | W | 753 | 12.990 | 62.108 | −25.006 | 1.00 | 33.01 | W |
| ATOM | 8936 | OH2 | WAT | W | 754 | 23.345 | 87.363 | −32.635 | 1.00 | 32.61 | W |
| ATOM | 8937 | OH2 | WAT | W | 755 | 66.469 | 81.830 | −14.265 | 1.00 | 24.46 | W |
| ATOM | 8938 | OH2 | WAT | W | 756 | 47.252 | 61.569 | −28.848 | 1.00 | 30.46 | W |
| ATOM | 8939 | OH2 | WAT | W | 757 | 52.546 | 71.147 | 5.890 | 1.00 | 33.46 | W |
| ATOM | 8940 | OH2 | WAT | W | 758 | 41.001 | 56.046 | −33.215 | 1.00 | 27.40 | W |
| ATOM | 8941 | OH2 | WAT | W | 759 | 39.617 | 39.925 | 19.990 | 1.00 | 31.20 | W |
| ATOM | 8942 | OH2 | WAT | W | 760 | 44.781 | 55.360 | 36.095 | 1.00 | 36.29 | W |
| ATOM | 8943 | OH2 | WAT | W | 761 | 13.955 | 62.545 | 14.561 | 1.00 | 24.95 | W |
| ATOM | 8944 | OH2 | WAT | W | 762 | 39.940 | 39.555 | −0.351 | 1.00 | 33.58 | W |
| ATOM | 8945 | OH2 | WAT | W | 763 | 32.665 | 69.642 | 22.088 | 1.00 | 26.34 | W |
| ATOM | 8946 | OH2 | WAT | W | 764 | 42.575 | 43.594 | −11.934 | 1.00 | 31.73 | W |
| ATOM | 8947 | OH2 | WAT | W | 765 | 26.998 | 41.795 | −27.476 | 1.00 | 34.79 | W |
| ATOM | 8948 | OH2 | WAT | W | 766 | 19.705 | 41.788 | −5.040 | 1.00 | 30.18 | W |
| ATOM | 8949 | OH2 | WAT | W | 767 | 13.729 | 60.851 | 7.587 | 1.00 | 31.12 | W |
| ATOM | 8950 | OH2 | WAT | W | 768 | 46.594 | 45.832 | 11.529 | 1.00 | 30.71 | W |
| ATOM | 8951 | OH2 | WAT | W | 769 | 43.004 | 68.714 | −30.001 | 1.00 | 34.08 | W |
| ATOM | 8952 | OH2 | WAT | W | 770 | 24.346 | 54.101 | −8.362 | 1.00 | 40.20 | W |
| ATOM | 8953 | OH2 | WAT | W | 771 | 47.715 | 70.196 | −16.599 | 1.00 | 29.28 | W |
| ATOM | 8954 | OH2 | WAT | W | 772 | 58.821 | 93.877 | −27.444 | 1.00 | 33.88 | W |
| ATOM | 8955 | OH2 | WAT | W | 773 | 31.148 | 79.112 | −42.939 | 1.00 | 36.00 | W |
| ATOM | 8956 | OH2 | WAT | W | 774 | 22.053 | 42.741 | −13.266 | 1.00 | 29.08 | W |
| ATOM | 8957 | OH2 | WAT | W | 775 | 52.877 | 92.345 | −23.218 | 1.00 | 28.25 | W |
| ATOM | 8958 | OH2 | WAT | W | 776 | 60.172 | 51.088 | 20.144 | 1.00 | 37.24 | W |
| ATOM | 8959 | OH2 | WAT | W | 777 | 60.950 | 56.059 | 1.983 | 1.00 | 32.34 | W |
| ATOM | 8960 | OH2 | WAT | W | 778 | 19.502 | 58.697 | −36.820 | 1.00 | 28.69 | W |
| ATOM | 8961 | OH2 | WAT | W | 779 | 30.076 | 50.066 | 12.361 | 1.00 | 66.40 | W |
| ATOM | 8962 | OH2 | WAT | W | 780 | 26.320 | 66.838 | 19.785 | 1.00 | 20.83 | W |
| ATOM | 8963 | OH2 | WAT | W | 781 | 12.032 | 41.651 | 19.833 | 1.00 | 27.28 | W |
| ATOM | 8964 | OH2 | WAT | W | 782 | 69.452 | 77.231 | −34.140 | 1.00 | 32.70 | W |
| ATOM | 8965 | OH2 | WAT | W | 783 | 16.602 | 43.039 | −2.678 | 1.00 | 24.99 | W |
| ATOM | 8966 | OH2 | WAT | W | 784 | 35.764 | 60.018 | −37.747 | 1.00 | 36.97 | W |
| ATOM | 8967 | OH2 | WAT | W | 785 | 33.876 | 66.348 | −42.439 | 1.00 | 39.91 | W |
| ATOM | 8968 | OH2 | WAT | W | 786 | 57.127 | 36.355 | −14.326 | 1.00 | 37.67 | W |
| ATOM | 8969 | OH2 | WAT | W | 787 | 37.130 | 37.609 | 2.903 | 1.00 | 39.84 | W |
| ATOM | 8970 | OH2 | WAT | W | 788 | 51.220 | 66.924 | 8.375 | 1.00 | 31.06 | W |
| ATOM | 8971 | OH2 | WAT | W | 789 | 10.804 | 51.718 | 21.423 | 1.00 | 33.84 | W |
| ATOM | 8972 | OH2 | WAT | W | 790 | 30.270 | 87.469 | −15.061 | 1.00 | 26.18 | W |
| ATOM | 8973 | OH2 | WAT | W | 791 | 41.988 | 55.052 | −17.332 | 1.00 | 31.54 | W |
| ATOM | 8974 | OH2 | WAT | W | 792 | 48.606 | 76.258 | 8.357 | 1.00 | 38.63 | W |
| ATOM | 8975 | OH2 | WAT | W | 793 | 29.552 | 75.900 | 10.796 | 1.00 | 22.83 | W |
| ATOM | 8976 | OH2 | WAT | W | 794 | 42.986 | 98.280 | −37.052 | 1.00 | 36.49 | W |
| ATOM | 8977 | OH2 | WAT | W | 795 | 23.446 | 66.228 | 9.839 | 1.00 | 36.96 | W |
| ATOM | 8978 | OH2 | WAT | W | 796 | 64.807 | 79.091 | −1.652 | 1.00 | 32.76 | W |
| ATOM | 8979 | OH2 | WAT | W | 797 | 43.476 | 40.507 | −22.892 | 1.00 | 31.16 | W |
| ATOM | 8980 | OH2 | WAT | W | 798 | 59.402 | 49.107 | 2.170 | 1.00 | 36.25 | W |
| ATOM | 8981 | OH2 | WAT | W | 799 | 68.966 | 41.741 | −17.222 | 1.00 | 33.61 | W |
| ATOM | 8982 | OH2 | WAT | W | 800 | 24.793 | 71.941 | −39.851 | 1.00 | 21.86 | W |
| ATOM | 8983 | OH2 | WAT | W | 801 | 23.767 | 48.580 | −24.246 | 1.00 | 34.92 | W |
| ATOM | 8984 | OH2 | WAT | W | 802 | 46.980 | 68.168 | −24.735 | 1.00 | 26.18 | W |
| ATOM | 8985 | OH2 | WAT | W | 803 | 53.458 | 53.195 | −29.972 | 1.00 | 28.69 | W |
| ATOM | 8986 | OH2 | WAT | W | 804 | 24.862 | 34.453 | 35.634 | 1.00 | 36.72 | W |
| ATOM | 8987 | OH2 | WAT | W | 805 | 13.428 | 52.674 | 25.889 | 1.00 | 33.75 | W |
| ATOM | 8988 | OH2 | WAT | W | 806 | 51.562 | 44.638 | 2.845 | 1.00 | 28.99 | W |
| ATOM | 8989 | OH2 | WAT | W | 807 | 21.377 | 55.670 | 39.671 | 1.00 | 40.73 | W |
| ATOM | 8990 | OH2 | WAT | W | 808 | 64.134 | 70.734 | −5.183 | 1.00 | 21.34 | W |
| ATOM | 8991 | OH2 | WAT | W | 809 | 46.972 | 89.853 | −23.163 | 1.00 | 38.24 | W |
| ATOM | 8992 | OH2 | WAT | W | 810 | 8.000 | 53.174 | −7.596 | 1.00 | 25.72 | W |
| ATOM | 8993 | OH2 | WAT | W | 811 | 22.177 | 80.927 | −42.182 | 1.00 | 30.13 | W |
| ATOM | 8994 | OH2 | WAT | W | 812 | 63.779 | 65.758 | −30.505 | 1.00 | 44.49 | W |
| ATOM | 8995 | OH2 | WAT | W | 813 | 18.366 | 48.706 | −19.763 | 1.00 | 34.11 | W |
| ATOM | 8996 | OH2 | WAT | W | 814 | 59.401 | 76.927 | −4.247 | 1.00 | 31.82 | W |
| ATOM | 8997 | OH2 | WAT | W | 815 | 21.046 | 84.135 | −12.381 | 1.00 | 30.70 | W |
| ATOM | 8998 | OH2 | WAT | W | 816 | 55.643 | 67.386 | 13.253 | 1.00 | 42.99 | W |
| ATOM | 8999 | OH2 | WAT | W | 817 | 19.135 | 52.415 | −31.528 | 1.00 | 43.38 | W |
| ATOM | 9000 | OH2 | WAT | W | 818 | 67.337 | 85.739 | −35.938 | 1.00 | 31.07 | W |
| ATOM | 9001 | OH2 | WAT | W | 819 | 29.637 | 75.151 | 22.301 | 1.00 | 26.35 | W |
| ATOM | 9002 | OH2 | WAT | W | 820 | 32.750 | 84.350 | 0.358 | 1.00 | 22.00 | W |
| ATOM | 9003 | OH2 | WAT | W | 821 | 45.598 | 65.481 | −28.648 | 1.00 | 31.79 | W |
| ATOM | 9004 | OH2 | WAT | W | 822 | 12.768 | 62.241 | −2.419 | 1.00 | 31.35 | W |
| ATOM | 9005 | OH2 | WAT | W | 823 | 25.799 | 63.445 | 13.810 | 1.00 | 32.36 | W |
| ATOM | 9006 | OH2 | WAT | W | 824 | 28.556 | 34.999 | 32.201 | 1.00 | 37.91 | W |
| ATOM | 9007 | OH2 | WAT | W | 825 | 36.020 | 68.674 | 23.466 | 1.00 | 35.59 | W |
| ATOM | 9008 | OH2 | WAT | W | 826 | 31.938 | 33.896 | 17.286 | 1.00 | 39.46 | W |
| ATOM | 9009 | OH2 | WAT | W | 827 | 41.647 | 84.318 | −2.417 | 1.00 | 41.96 | W |
| ATOM | 9010 | OH2 | WAT | W | 828 | 40.024 | 100.332 | −31.852 | 1.00 | 43.82 | W |
| ATOM | 9011 | OH2 | WAT | W | 829 | 28.695 | 63.602 | 10.619 | 1.00 | 15.02 | W |
| ATOM | 9012 | OH2 | WAT | W | 830 | 54.701 | 82.602 | −44.566 | 1.00 | 41.37 | W |
| ATOM | 9013 | OH2 | WAT | W | 831 | 69.916 | 53.723 | −10.485 | 1.00 | 30.03 | W |
| ATOM | 9014 | OH2 | WAT | W | 832 | 36.974 | 79.509 | −2.521 | 1.00 | 47.01 | W |
| ATOM | 9015 | OH2 | WAT | W | 833 | 12.230 | 64.667 | −12.621 | 1.00 | 35.16 | W |
| ATOM | 9016 | OH2 | WAT | W | 834 | 39.082 | 50.367 | 36.260 | 1.00 | 40.49 | W |
| ATOM | 9017 | OH2 | WAT | W | 835 | 27.965 | 44.799 | 38.117 | 1.00 | 30.19 | W |
| ATOM | 9018 | OH2 | WAT | W | 836 | 27.787 | 38.780 | −22.542 | 1.00 | 37.36 | W |
| ATOM | 9019 | OH2 | WAT | W | 837 | 72.305 | 95.801 | −31.273 | 1.00 | 33.36 | W |
| ATOM | 9020 | OH2 | WAT | W | 838 | 76.326 | 47.087 | −11.653 | 1.00 | 33.77 | W |
| ATOM | 9021 | OH2 | WAT | W | 839 | 21.477 | 49.084 | −38.808 | 1.00 | 37.70 | W |
| ATOM | 9022 | OH2 | WAT | W | 840 | 23.074 | 75.206 | −30.020 | 1.00 | 36.46 | W |
| ATOM | 9023 | OH2 | WAT | W | 841 | 20.982 | 31.706 | 31.644 | 1.00 | 41.44 | W |
| ATOM | 9024 | OH2 | WAT | W | 842 | 29.441 | 42.198 | −28.737 | 1.00 | 51.18 | W |
| ATOM | 9025 | OH2 | WAT | W | 843 | 42.659 | 47.355 | −27.313 | 1.00 | 40.92 | W |
| ATOM | 9026 | OH2 | WAT | W | 844 | 55.868 | 51.887 | 27.288 | 1.00 | 31.92 | W |
| ATOM | 9027 | OH2 | WAT | W | 845 | 31.328 | 58.787 | −40.674 | 1.00 | 50.82 | W |
| ATOM | 9028 | OH2 | WAT | W | 846 | 18.450 | 43.148 | 27.316 | 1.00 | 29.94 | W |
| ATOM | 9029 | OH2 | WAT | W | 847 | 62.202 | 81.791 | −43.971 | 1.00 | 45.83 | W |
| ATOM | 9030 | OH2 | WAT | W | 848 | 7.623 | 54.625 | 17.516 | 1.00 | 32.61 | W |
| ATOM | 9031 | OH2 | WAT | W | 849 | 18.083 | 41.899 | 25.153 | 1.00 | 37.23 | W |
| ATOM | 9032 | OH2 | WAT | W | 850 | 27.414 | 82.738 | 5.782 | 1.00 | 42.08 | W |
| ATOM | 9033 | OH2 | WAT | W | 851 | 38.762 | 53.294 | 1.928 | 1.00 | 35.13 | W |
| ATOM | 9034 | OH2 | WAT | W | 852 | 11.930 | 67.467 | 2.674 | 1.00 | 46.11 | W |
| ATOM | 9035 | OH2 | WAT | W | 853 | 4.368 | 56.741 | −6.545 | 1.00 | 31.50 | W |
| ATOM | 9036 | OH2 | WAT | W | 854 | 22.233 | 77.054 | 8.620 | 1.00 | 23.51 | W |
| ATOM | 9037 | OH2 | WAT | W | 855 | 25.877 | 88.243 | −42.821 | 1.00 | 39.96 | W |
| ATOM | 9038 | OH2 | WAT | W | 856 | 10.521 | 62.691 | −12.058 | 1.00 | 35.57 | W |
| ATOM | 9039 | OH2 | WAT | W | 857 | 44.573 | 72.271 | −44.611 | 1.00 | 40.66 | W |
| ATOM | 9040 | OH2 | WAT | W | 858 | 37.290 | 40.005 | 26.383 | 1.00 | 42.44 | W |
| ATOM | 9041 | OH2 | WAT | W | 859 | 23.430 | 89.005 | −38.011 | 1.00 | 37.56 | W |
| ATOM | 9042 | OH2 | WAT | W | 860 | 70.109 | 62.809 | −24.295 | 1.00 | 41.47 | W |
| ATOM | 9043 | OH2 | WAT | W | 861 | 23.013 | 64.466 | 18.350 | 1.00 | 31.90 | W |
| ATOM | 9044 | OH2 | WAT | W | 862 | 67.076 | 38.608 | −9.606 | 1.00 | 27.89 | W |
| ATOM | 9045 | OH2 | WAT | W | 863 | 65.523 | 87.855 | −41.037 | 1.00 | 41.58 | W |
| ATOM | 9046 | OH2 | WAT | W | 864 | 13.958 | 43.645 | 28.956 | 1.00 | 43.91 | W |
| ATOM | 9047 | OH2 | WAT | W | 865 | 13.521 | 48.352 | 1.213 | 1.00 | 37.81 | W |
| ATOM | 9048 | OH2 | WAT | W | 866 | 55.482 | 69.618 | −38.014 | 1.00 | 38.32 | W |
| ATOM | 9049 | OH2 | WAT | W | 867 | 26.983 | 47.671 | −23.958 | 1.00 | 34.58 | W |
| ATOM | 9050 | OH2 | WAT | W | 868 | 69.691 | 82.300 | −33.929 | 1.00 | 31.58 | W |
| ATOM | 9051 | OH2 | WAT | W | 869 | 12.125 | 62.282 | 22.712 | 1.00 | 41.22 | W |
| ATOM | 9052 | OH2 | WAT | W | 870 | 30.789 | 29.802 | 5.467 | 1.00 | 41.46 | W |
| ATOM | 9053 | OH2 | WAT | W | 871 | 19.363 | 36.418 | 22.415 | 1.00 | 43.26 | W |
| ATOM | 9054 | OH2 | WAT | W | 872 | 27.591 | 86.553 | −14.741 | 1.00 | 26.30 | W |
| ATOM | 9055 | OH2 | WAT | W | 873 | 66.334 | 50.435 | 0.529 | 1.00 | 37.46 | W |
| ATOM | 9056 | OH2 | WAT | W | 874 | 28.581 | 64.849 | 14.838 | 1.00 | 19.58 | W |
| ATOM | 9057 | OH2 | WAT | W | 875 | 52.012 | 60.259 | 28.144 | 1.00 | 32.70 | W |
| ATOM | 9058 | OH2 | WAT | W | 876 | 52.497 | 72.590 | 18.443 | 1.00 | 37.71 | W |
| ATOM | 9059 | OH2 | WAT | W | 877 | 47.030 | 75.056 | −26.288 | 1.00 | 33.47 | W |
| ATOM | 9060 | OH2 | WAT | W | 878 | 65.356 | 81.557 | −4.607 | 1.00 | 35.86 | W |
| ATOM | 9061 | OH2 | WAT | W | 879 | 48.561 | 72.320 | −26.150 | 1.00 | 35.66 | W |
| ATOM | 9062 | OH2 | WAT | W | 880 | 59.811 | 68.887 | −38.775 | 1.00 | 39.50 | W |
| ATOM | 9063 | OH2 | WAT | W | 881 | 22.852 | 85.136 | −16.949 | 1.00 | 40.06 | W |
| ATOM | 9064 | OH2 | WAT | W | 882 | 25.845 | 76.358 | −38.701 | 1.00 | 38.63 | W |
| ATOM | 9065 | OH2 | WAT | W | 883 | 20.344 | 74.115 | −29.032 | 1.00 | 47.88 | W |
| ATOM | 9066 | OH2 | WAT | W | 884 | 59.348 | 55.599 | 27.039 | 1.00 | 38.54 | W |
| ATOM | 9067 | OH2 | WAT | W | 885 | 44.363 | 87.562 | −15.598 | 1.00 | 46.87 | W |
| ATOM | 9068 | OH2 | WAT | W | 886 | 63.961 | 77.047 | −40.514 | 1.00 | 35.09 | W |
| ATOM | 9069 | OH2 | WAT | W | 887 | 14.182 | 58.399 | 16.314 | 1.00 | 20.35 | W |
| ATOM | 9070 | OH2 | WAT | W | 888 | 14.005 | 57.801 | 18.961 | 1.00 | 16.96 | W |
| ATOM | 9071 | OH2 | WAT | W | 889 | 24.482 | 63.758 | 11.558 | 1.00 | 40.41 | W |
| ATOM | 9072 | OH2 | WAT | W | 890 | 28.177 | 39.811 | 11.260 | 1.00 | 14.10 | W |
| ATOM | 9073 | OH2 | WAT | W | 891 | 28.968 | 82.073 | −35.705 | 1.00 | 23.63 | W |
| ATOM | 9074 | OH2 | WAT | W | 892 | 12.332 | 55.927 | −15.108 | 1.00 | 23.12 | W |
| ATOM | 9075 | OH2 | WAT | W | 893 | 81.492 | 67.229 | −13.707 | 1.00 | 21.97 | W |
| ATOM | 9076 | OH2 | WAT | W | 894 | 58.926 | 58.066 | 0.988 | 1.00 | 25.28 | W |
| ATOM | 9077 | OH2 | WAT | W | 895 | 49.226 | 68.465 | 6.744 | 1.00 | 21.20 | W |
| ATOM | 9078 | OH2 | WAT | W | 896 | 68.450 | 79.680 | −28.527 | 1.00 | 24.55 | W |
| ATOM | 9079 | OH2 | WAT | W | 897 | 26.056 | 32.722 | 28.695 | 1.00 | 26.11 | W |
| ATOM | 9080 | OH2 | WAT | W | 898 | 32.819 | 69.547 | 24.596 | 1.00 | 29.62 | W |
| ATOM | 9081 | OH2 | WAT | W | 899 | 28.018 | 102.798 | −19.150 | 1.00 | 27.36 | W |
| ATOM | 9082 | OH2 | WAT | W | 900 | 71.696 | 73.995 | −29.144 | 1.00 | 33.51 | W |
| ATOM | 9083 | OH2 | WAT | W | 901 | 79.298 | 50.626 | −8.882 | 1.00 | 32.39 | W |
| ATOM | 9084 | OH2 | WAT | W | 902 | 37.121 | 83.790 | −3.946 | 1.00 | 25.44 | W |
| ATOM | 9085 | OH2 | WAT | W | 903 | 59.411 | 52.085 | 2.463 | 1.00 | 28.12 | W |
| ATOM | 9086 | OH2 | WAT | W | 904 | 19.832 | 84.479 | −18.505 | 1.00 | 31.53 | W |
| ATOM | 9087 | OH2 | WAT | W | 905 | 43.802 | 79.359 | −21.515 | 1.00 | 30.31 | W |
| ATOM | 9088 | OH2 | WAT | W | 906 | 57.911 | 52.910 | 26.179 | 1.00 | 36.06 | W |
| ATOM | 9089 | OH2 | WAT | W | 907 | 16.938 | 57.510 | −36.365 | 1.00 | 30.40 | W |
| ATOM | 9090 | OH2 | WAT | W | 908 | 46.724 | 42.921 | 9.610 | 1.00 | 32.35 | W |
| ATOM | 9091 | OH2 | WAT | W | 909 | 27.272 | 72.059 | 33.936 | 1.00 | 39.20 | W |
| ATOM | 9092 | OH2 | WAT | W | 910 | 7.389 | 54.456 | −10.032 | 1.00 | 24.47 | W |
| ATOM | 9093 | OH2 | WAT | W | 911 | 24.568 | 52.451 | 43.481 | 1.00 | 47.08 | W |
| ATOM | 9094 | OH2 | WAT | W | 912 | 49.865 | 47.024 | −30.511 | 1.00 | 33.76 | W |
| ATOM | 9095 | OH2 | WAT | W | 913 | 42.658 | 78.817 | −29.502 | 1.00 | 35.03 | W |
| ATOM | 9096 | OH2 | WAT | W | 914 | 27.537 | 88.946 | −12.482 | 1.00 | 31.40 | W |
| ATOM | 9097 | OH2 | WAT | W | 915 | 56.678 | 90.189 | −43.614 | 1.00 | 35.75 | W |
| ATOM | 9098 | OH2 | WAT | W | 916 | 14.006 | 44.947 | 26.811 | 1.00 | 37.06 | W |
| ATOM | 9099 | OH2 | WAT | W | 917 | 69.590 | 84.000 | −36.011 | 1.00 | 33.87 | W |
| ATOM | 9100 | OH2 | WAT | W | 918 | 57.990 | 93.503 | −25.101 | 1.00 | 32.28 | W |
| ATOM | 9101 | OH2 | WAT | W | 919 | 64.754 | 70.419 | −8.091 | 1.00 | 53.24 | W |
| ATOM | 9102 | OH2 | WAT | W | 920 | 46.084 | 39.866 | −0.054 | 1.00 | 29.17 | W |
| ATOM | 9103 | OH2 | WAT | W | 921 | 37.055 | 37.381 | 24.919 | 1.00 | 36.57 | W |
| ATOM | 9104 | OH2 | WAT | W | 922 | 52.320 | 68.585 | 4.797 | 1.00 | 34.76 | W |
| ATOM | 9105 | OH2 | WAT | W | 923 | 17.923 | 51.020 | 38.034 | 1.00 | 35.67 | W |
| ATOM | 9106 | OH2 | WAT | W | 924 | 15.320 | 77.244 | −13.699 | 1.00 | 35.62 | W |
| ATOM | 9107 | OH2 | WAT | W | 925 | 20.069 | 39.376 | −12.317 | 1.00 | 37.80 | W |
| ATOM | 9108 | OH2 | WAT | W | 926 | 49.621 | 45.620 | 30.973 | 1.00 | 30.61 | W |
| ATOM | 9109 | OH2 | WAT | W | 927 | 46.954 | 72.541 | −17.610 | 1.00 | 31.84 | W |
| ATOM | 9110 | OH2 | WAT | W | 928 | 70.522 | 78.290 | −25.994 | 1.00 | 38.39 | W |
| ATOM | 9111 | OH2 | WAT | W | 929 | 58.551 | 52.895 | 4.627 | 1.00 | 29.74 | W |
| ATOM | 9112 | OH2 | WAT | W | 930 | 35.513 | 55.491 | −34.759 | 1.00 | 41.24 | W |
| ATOM | 9113 | OH2 | WAT | W | 931 | 41.558 | 82.881 | −28.845 | 1.00 | 40.90 | W |
| ATOM | 9114 | OH2 | WAT | W | 932 | 48.127 | 66.178 | 26.914 | 1.00 | 36.07 | W |
| ATOM | 9115 | OH2 | WAT | W | 933 | 27.690 | 67.682 | 11.659 | 1.00 | 24.50 | W |
| ATOM | 9116 | OH2 | WAT | W | 934 | 39.804 | 82.006 | −21.936 | 1.00 | 34.34 | W |
| ATOM | 9117 | OH2 | WAT | W | 935 | 22.224 | 65.770 | 32.279 | 1.00 | 31.60 | W |
| ATOM | 9118 | OH2 | WAT | W | 936 | 58.814 | 48.750 | 18.909 | 1.00 | 33.30 | W |
| ATOM | 9119 | OH2 | WAT | W | 937 | 31.849 | 80.994 | 7.175 | 1.00 | 45.53 | W |
| ATOM | 9120 | OH2 | WAT | W | 938 | 50.363 | 44.965 | −24.802 | 1.00 | 36.04 | W |
| ATOM | 9121 | OH2 | WAT | W | 939 | 62.526 | 60.736 | 0.451 | 1.00 | 35.47 | W |
| ATOM | 9122 | OH2 | WAT | W | 940 | 67.854 | 75.409 | −35.151 | 1.00 | 39.71 | W |
| ATOM | 9123 | OH2 | WAT | W | 941 | 23.138 | 39.427 | −15.567 | 1.00 | 39.15 | W |
| ATOM | 9124 | OH2 | WAT | W | 942 | 35.976 | 94.035 | −29.929 | 1.00 | 35.78 | W |
| ATOM | 9125 | OH2 | WAT | W | 943 | 73.983 | 80.173 | −11.072 | 1.00 | 35.65 | W |
| ATOM | 9126 | OH2 | WAT | W | 944 | 41.088 | 81.781 | −3.482 | 1.00 | 41.37 | W |
| ATOM | 9127 | OH2 | WAT | W | 945 | 13.020 | 63.025 | 12.074 | 1.00 | 43.97 | W |
| ATOM | 9128 | OH2 | WAT | W | 946 | 56.714 | 76.086 | 0.096 | 1.00 | 33.32 | W |
| ATOM | 9129 | OH2 | WAT | W | 947 | 74.317 | 52.113 | 1.536 | 1.00 | 36.02 | W |
| ATOM | 9130 | OH2 | WAT | W | 948 | 22.014 | 61.465 | −34.827 | 1.00 | 31.87 | W |
| ATOM | 9131 | OH2 | WAT | W | 949 | 77.383 | 65.431 | −15.067 | 1.00 | 27.69 | W |
| ATOM | 9132 | OH2 | WAT | W | 950 | 17.693 | 41.868 | 32.102 | 1.00 | 47.71 | W |
| ATOM | 9133 | OH2 | WAT | W | 951 | 10.258 | 63.048 | −15.051 | 1.00 | 35.09 | W |
| ATOM | 9134 | OH2 | WAT | W | 952 | 45.905 | 79.926 | 4.885 | 1.00 | 35.52 | W |
| ATOM | 9135 | OH2 | WAT | W | 953 | 14.632 | 46.962 | 32.202 | 1.00 | 28.79 | W |
| ATOM | 9136 | OH2 | WAT | W | 954 | 34.451 | 77.305 | 12.190 | 1.00 | 30.17 | W |
| ATOM | 9137 | OH2 | WAT | W | 955 | 47.521 | 40.270 | 5.969 | 1.00 | 34.52 | W |
| ATOM | 9138 | OH2 | WAT | W | 956 | 18.766 | 39.515 | −2.957 | 1.00 | 38.68 | W |
| ATOM | 9139 | OH2 | WAT | W | 957 | 25.886 | 59.107 | −38.846 | 1.00 | 37.93 | W |
| ATOM | 9140 | OH2 | WAT | W | 958 | 10.640 | 57.926 | −20.969 | 1.00 | 40.59 | W |
| ATOM | 9141 | OH2 | WAT | W | 959 | 8.881 | 56.896 | 16.376 | 1.00 | 46.16 | W |
| ATOM | 9142 | OH2 | WAT | W | 960 | 78.130 | 72.254 | −10.221 | 1.00 | 30.28 | W |
| ATOM | 9143 | OH2 | WAT | W | 961 | 23.884 | 82.876 | −3.966 | 1.00 | 52.89 | W |
| ATOM | 9144 | OH2 | WAT | W | 962 | 44.483 | 40.961 | 7.504 | 1.00 | 44.52 | W |
| ATOM | 9145 | OH2 | WAT | W | 963 | 35.403 | 62.493 | 33.962 | 1.00 | 31.32 | W |
| ATOM | 9146 | OH2 | WAT | W | 964 | 27.262 | 90.077 | −31.713 | 1.00 | 39.79 | W |
| ATOM | 9147 | OH2 | WAT | W | 965 | 74.769 | 77.373 | −20.732 | 1.00 | 39.11 | W |
| ATOM | 9148 | OH2 | WAT | W | 966 | 49.100 | 42.041 | 8.300 | 1.00 | 33.85 | W |
| ATOM | 9149 | OH2 | WAT | W | 967 | 44.902 | 92.910 | −25.044 | 1.00 | 36.65 | W |
| ATOM | 9150 | OH2 | WAT | W | 968 | 52.476 | 49.145 | 25.620 | 1.00 | 35.34 | W |
| ATOM | 9151 | OH2 | WAT | W | 969 | 9.649 | 53.663 | −12.194 | 1.00 | 30.57 | W |
| ATOM | 9152 | OH2 | WAT | W | 970 | 58.733 | 51.116 | 14.538 | 1.00 | 47.36 | W |
| ATOM | 9153 | OH2 | WAT | W | 971 | 51.884 | 50.452 | 27.694 | 1.00 | 38.68 | W |
| ATOM | 9154 | OH2 | WAT | W | 972 | 25.020 | 89.138 | −25.139 | 1.00 | 38.86 | W |
| ATOM | 9155 | OH2 | WAT | W | 973 | 7.521 | 58.222 | 0.201 | 1.00 | 37.34 | W |
| ATOM | 9156 | OH2 | WAT | W | 974 | 11.524 | 58.491 | 19.987 | 1.00 | 37.19 | W |
| ATOM | 9157 | OH2 | WAT | W | 975 | 18.598 | 60.943 | −37.781 | 1.00 | 25.28 | W |
| ATOM | 9158 | OH2 | WAT | W | 976 | 46.002 | 60.113 | −31.315 | 1.00 | 37.62 | W |
| ATOM | 9159 | OH2 | WAT | W | 977 | 5.368 | 57.731 | 6.783 | 1.00 | 33.43 | W |
| ATOM | 9160 | OH2 | WAT | W | 978 | 13.342 | 66.496 | −11.623 | 1.00 | 59.42 | W |
| ATOM | 9161 | OH2 | WAT | W | 979 | 47.205 | 81.657 | −17.119 | 1.00 | 33.31 | W |
| ATOM | 9162 | OH2 | WAT | W | 981 | 29.413 | 65.677 | 12.426 | 1.00 | 21.92 | W |
| ATOM | 9163 | OH2 | WAT | W | 982 | 28.559 | 69.915 | 12.822 | 1.00 | 31.51 | W |
| ATOM | 9164 | OH2 | WAT | W | 983 | 33.312 | 68.983 | 16.916 | 1.00 | 38.02 | W |
| ATOM | 9165 | C1 | NAG | C | 1 | 58.321 | 45.027 | 12.880 | 1.00 | 46.25 | C |
| ATOM | 9166 | C2 | NAG | C | 1 | 59.553 | 44.726 | 13.744 | 1.00 | 48.64 | C |
| ATOM | 9167 | N2 | NAG | C | 1 | 60.611 | 45.671 | 13.441 | 1.00 | 50.02 | C |
| ATOM | 9168 | C7 | NAG | C | 1 | 60.818 | 46.715 | 14.237 | 1.00 | 51.53 | C |
| ATOM | 9169 | O7 | NAG | C | 1 | 60.267 | 47.803 | 14.068 | 1.00 | 53.37 | C |
| ATOM | 9170 | C8 | NAG | C | 1 | 61.782 | 46.528 | 15.397 | 1.00 | 51.81 | C |
| ATOM | 9171 | C3 | NAG | C | 1 | 60.046 | 43.300 | 13.495 | 1.00 | 49.65 | C |
| ATOM | 9172 | O3 | NAG | C | 1 | 61.101 | 42.997 | 14.395 | 1.00 | 50.85 | C |
| ATOM | 9173 | C4 | NAG | C | 1 | 58.905 | 42.304 | 13.689 | 1.00 | 49.25 | C |
| ATOM | 9174 | O4 | NAG | C | 1 | 59.345 | 40.999 | 13.344 | 1.00 | 50.57 | C |
| ATOM | 9175 | C5 | NAG | C | 1 | 57.716 | 42.702 | 12.810 | 1.00 | 49.13 | C |
| ATOM | 9176 | O5 | NAG | C | 1 | 57.295 | 44.050 | 13.123 | 1.00 | 47.36 | C |
| ATOM | 9177 | C6 | NAG | C | 1 | 56.518 | 41.797 | 13.024 | 1.00 | 48.96 | C |
| ATOM | 9178 | O6 | NAG | C | 1 | 55.511 | 42.038 | 12.052 | 1.00 | 50.02 | C |
| ATOM | 9179 | C1 | SWA | | 1 | 31.064 | 66.873 | 6.079 | 1.00 | 9.68 | |
| ATOM | 9180 | O1 | SWA | | 1 | 31.597 | 68.032 | 5.416 | 1.00 | 11.09 | |
| ATOM | 9181 | C3 | SWA | | 1 | 31.295 | 67.013 | 7.615 | 1.00 | 10.48 | |
| ATOM | 9182 | N4 | SWA | | 1 | 30.738 | 65.836 | 8.320 | 1.00 | 10.85 | |
| ATOM | 9183 | C5 | SWA | | 1 | 29.276 | 65.603 | 8.133 | 1.00 | 10.14 | |
| ATOM | 9184 | C6 | SWA | | 1 | 28.973 | 65.471 | 6.610 | 1.00 | 9.14 | |
| ATOM | 9185 | C2 | SWA | | 1 | 29.531 | 66.700 | 5.827 | 1.00 | 9.59 | |
| ATOM | 9186 | C9 | SWA | | 1 | 31.268 | 65.873 | 9.682 | 1.00 | 11.70 | |
| ATOM | 9187 | C8 | SWA | | 1 | 32.681 | 66.486 | 9.558 | 1.00 | 11.30 | |
| ATOM | 9188 | O13 | SWA | | 1 | 33.681 | 65.511 | 9.836 | 1.00 | 11.34 | |
| ATOM | 9189 | C7 | SWA | | 1 | 32.764 | 67.083 | 8.112 | 1.00 | 10.44 | |
| ATOM | 9190 | O11 | SWA | | 1 | 33.674 | 66.300 | 7.314 | 1.00 | 10.67 | |
| ATOM | 9191 | C1 | MPD | M | 1 | 14.797 | 61.266 | 10.322 | 1.00 | 23.09 | M |
| ATOM | 9192 | C2 | MPD | M | 1 | 16.264 | 61.479 | 10.614 | 1.00 | 23.60 | M |
| ATOM | 9193 | O2 | MPD | M | 1 | 16.876 | 60.330 | 9.851 | 1.00 | 24.23 | M |
| ATOM | 9194 | CM | MPD | M | 1 | 17.075 | 62.611 | 10.000 | 1.00 | 24.36 | M |
| ATOM | 9195 | C3 | MPD | M | 1 | 16.492 | 61.271 | 12.125 | 1.00 | 23.19 | M |
| ATOM | 9196 | C4 | MPD | M | 1 | 17.813 | 60.945 | 12.649 | 1.00 | 23.12 | M |
| ATOM | 9197 | O4 | MPD | M | 1 | 17.580 | 59.936 | 13.627 | 1.00 | 22.00 | M |
| ATOM | 9198 | C5 | MPD | N | 1 | 18.337 | 62.222 | 13.387 | 1.00 | 22.61 | M |
| ATOM | 9199 | ZN | ZN | Z | 1 | 34.561 | 64.335 | 8.062 | 1.00 | 15.34 | Z |

TABLE 3
From initial analysis of the crystal structure, based on the location of residues known to be
involved in catalysis, the active site of the enzyme is located within a region composed of the
following residues:
Structural Results: Active Site

| | | | | | | | | | | | |
|---|---|---|---|---|---|---|---|---|---|---|---|
| GLY | 60 | VAL | 61 | TRP | 62 | LYS | 63 | GLN | 64 | GLY | 65 |
| ILE | 68 | VAL | 83 | PHE | 84 | VAL | 85 | VAL | 86 | PRO | 87 |
| HIS | 88 | SER | 89 | HIS | 90 | ASN | 91 | ASP | 92 | TRP | 95 |
| ILE | 110 | met | 124 | PHE | 126 | ILE | 127 | TRP | 128 | ala | 129 |
| GLU | 130 | VAL | 161 | MET | 167 | TRP | 201 | ALA | 202 | ILE | 203 |
| ASP | 204 | PRO | 205 | PHE | 206 | GLY | 207 | HIS | 208 | LEU | 225 |
| ILE | 226 | GLN | 227 | ARG | 228 | thr | 229 | TYR | 231 | LYS | 234 |
| LEU | 243 | HIS | 262 | MET | 263 | MET | 264 | PRO | 265 | PHE | 266 |
| TYR | 267 | SER | 268 | TYR | 269 | ASP | 270 | ILE | 271 | PRO | 272 |
| HIS | 273 | THR | 274 | CYS | 275 | GLY | 276 | PRO | 277 | ASP | 278 |
| PRO | 279 | LYS | 280 | val | 281 | CYS | 282 | CYS | 283 | GLN | 284 |
| PHE | 285 | ASP | 286 | PHE | 287 | LYS | 288 | ARG | 289 | met | 290 |
| phe | 293 | gly | 294 | leu | 295 | ser | 296 | CYS | 297 | PRO | 298 |
| TRP | 299 | lys | 300 | VAL | 301 | PRO | 302 | PRO | 303 | leu | 317 |
| LEU | 318 | GLN | 321 | trp | 322 | LYS | 324 | LYS | 325 | ala | 326 |
| LEU | 328 | tyr | 329 | LEU | 334 | LEU | 335 | ile | 336 | PRO | 337 |
| LEU | 338 | GLY | 339 | ASP | 340 | ASP | 341 | PHE | 342 | ARG | 343 |
| phe | 344 | lys | 345 | GLU | 349 | val | 352 | GLN | 353 | arg | 354 |
| TYR | 357 | LEU | 360 | PHE | 361 | PHE | 376 | GLY | 377 | LEU | 379 |
| TYR | 407 | ALA | 408 | ASP | 409 | ARG | 410 | trp | 415 | HIS | 471 |
| ASP | 472 | TYR | 727 | ASP | 874 | glu | 875 | ARG | 876 | GLY | 877 |
| LEU | 878 | | | | | | | | | | |

Residues in lower case are not identical in the human Golgi ManII sequence, though many of those are conservative substitutions. In this sphere of 15 Å around the catalytic center, there are only 21 non-identities among 121 residues, indicating that the human active site will be essentially identical to that observed for the Drosophila structure.

The numbering system in this and the accompanying Figures corresponds to the protein expressed in the system described herein, not to full length ManII.

TABLE 4
Intermolecular Interactions at
an Active Site of a Mannosidase II Swainsonine Complex.

| | | | Distance | |
|---|---|---|---|---|
| | Swainsonine | Enzyme | Between | Atomic |
| No. of Atomic | Atomic | Atomic | Atomic | Interaction |
| Interaction | Contact | Contact | Contacts | Property |
| 1 | O1 | His 471 NE2 | 3.3 | HB |
| 2 | N | Asp 204 OD1 | 2.9 | HB |
| 3 | O2 | Asp 341 OD2 | 3.4 | HB |
| 4 | O2 | His 90 NE2 | 3.1 | HB |
| 5 | O2 | Asp 92 OD1 | 2.9 | HB |
| 6 | O8 | Asp 472 OD1 | 2.53 | HB |
| 7 | Ring | Phe 206 ring | 3.55 | VW |
| 8 | O8 | Tyr 727 OH | 2.6 | HB |
| 9 | ring | Trp 95 | 3.7 | VW |

HB: hydrogen bond interaction
VW Van der Waals

TABLE 5
Crystallographic Refinement Statistics for the Native Drosophila Gogli
Mannosidase II

>>> input coordinates: dgm12_ann_1bi.pdb
>>> molecular structure file: dgm12gen.mtf
>>> parameter file 1 : CNS_TOPPAR:protein_rep.param
>>> parameter file 2 : CNS_TOPPAR:water_rep.param
>>> parameter file 3 : CNS_TOPPAR:ion.param
>>> parameter file 4 : trs.par
>>> parameter file 5 : mpdnew2.par
>>> parameter file 6 : cis_peptide.param
>>> parameter file 7 : CNS_TOPPAR:carbohydrate.param
>>> reflection file= ../semetHiR.cv
>>> spacegroup: P2 (1) 2 (1) 2 (1)
>>> cell dimensions: a= 68.865 b= 109.718 c= 138.599 alpha= 90 beta= 90
gamma= 90
>>> current wa= 0.311868 for target= mif
>>> ncs= none
>>> initial B-factor correction applied to fobs :
>>> B11= 0.587 B22= −0.754 B33= 0.167
>>> B12= 0.000 B13= 0.000 B23= 0.000
>>> B-factor correction applied to coordinate array B: 0.088
>>> B-correction resolution: 6.0 - 1.4
>>> bulk solvent: density level= 0.359928 e/A\hat{ }3, B-factor= 43.085 A\hat{ }2
=================================== summary
==================================
resolution range: 500.0 - 1.4 A

| R-values: | |
|---|---|
| initial | r= 0.2348 free_r= 0.2361 |
| after B-factor and/or bulk solvent correction | r= 0.1931 free_r= 0.2105 |

Monitor for target “mlf” is R-value :

working set= 0.1931 test set= 0.2105

| | luzzati coordinate error | (5.0 - 1.4 A ): | 0.16 A |
|---|---|---|---|
| cross-validated luzzati coordinate error | | (5.0 - 1.4 A ): | 0.18 A |
| | sigmaa coordinate error | (5.0 - 1.4 A ): | 0.08 A |

| | | |
|---|---|---|
| cross-validated sigmaa coordinate error | (5.0 - 1.4 A ): | 0.10 A |

rmsd bonds= 0.004763 with 2 bond violations > 0.05
rmsd angles= 1.32400 with 32 angle violations > 8.0
rmsd dihedrals= 24.22585 with 4 angle violations > 60.0
rmsd improper= 0.80790 with 38 angle violations > 3.0
================================== B-factors
=================================
average B-factor= 15.7963
minimum B-factor= 4.5681
maximum B-factor= 57.2481
B rmsd for bonded mainchain atoms= 0.660
B rmsd for bonded sidechain atoms= 1.229
B rmsd for angle mainchain atoms= 1.131
B rmsd for angle sidechain atoms= 1.777
current rweight= 0.22677665
================================ diffraction data
============================
reflections with |Fobs|/sigma_F < 0.0 rejected
reflections with |Fobs| > 10000 * rms(Fobs) rejected

| | |
|---|---|
| theoretical total number of ref1. in resol. range: | 206243 ( 100.0 % ) |
| number of unobserved reflections (no entry or \|F\|=0): | 59797 ( 29.0 % ) |
| number of reflections rejected: | 0 ( 0.0 % ) |
| total number of reflections used: | 146446 ( 71.0 % ) |
| number of reflections in working set: | 139067 ( 67.4 % ) |
| number of reflections in test set: | 7379 ( 3.6 % ) |

=======> completeness

Test set (test = 1):
#bin | resolution range | #ref1 |

| | | | | |
|---|---|---|---|---|
| 1 | 3.02 | 500.01 | 1113 | 0.0521 |
| 2 | 2.39 | 3.02 | 1042 | 0.0501 |
| 3 | 2.09 | 2.39 | 1048 | 0.0508 |
| 4 | 1.90 | 2.09 | 1000 | 0.0485 |
| 5 | 1.76 | 1.90 | 989 | 0.0482 |
| 6 | 1.66 | 1.76 | 822 | 0.0401 |
| 7 | 1.58 | 1.66 | 584 | 0.0285 |
| 8 | 1.51 | 1.58 | 419 | 0.0205 |
| 9 | 1.45 | 1.51 | 265 | 0.0130 |
| 10 | 1.40 | 1.45 | 97 | 0.0048 |

Working set:
#bin | resolution range | #ref1 |

| | | | | |
|---|---|---|---|---|
| 1 | 3.02 | 500.01 | 20201 | 0.9460 |
| 2 | 2.39 | 3.02 | 19732 | 0.9491 |
| 3 | 2.09 | 2.39 | 19506 | 0.9447 |
| 4 | 1.90 | 2.09 | 19199 | 0.9316 |
| 5 | 1.76 | 1.90 | 18521 | 0.9019 |
| 6 | 1.66 | 1.76 | 15253 | 0.7434 |
| 7 | 1.58 | 1.66 | 11380 | 0.5551 |
| 8 | 1.51 | 1.58 | 8211 | 0.4018 |
| 9 | 1.45 | 1.51 | 5065 | 0.2477 |
| 10 | 1.40 | 1.45 | 1999 | 0.0980 |

================================= R-values
===================================
=======> R-values with |Fobs|/sigma cutoff= 0.0

Test set (test = 1):
#bin | resolution range | #ref1 |

| | | | | |
|---|---|---|---|---|
| 1 | 3.02 | 500.01 | 1113 | 0.1999 |
| 2 | 2.39 | 3.02 | 1042 | 0.2110 |
| 3 | 2.09 | 2.39 | 1048 | 0.2083 |
| 4 | 1.90 | 2.09 | 1000 | 0.2137 |
| 5 | 1.76 | 1.90 | 989 | 0.2244 |
| 6 | 1.66 | 1.76 | 822 | 0.2302 |
| 7 | 1.58 | 1.66 | 584 | 0.2275 |
| 8 | 1.51 | 1.58 | 419 | 0.2363 |
| 9 | 1.45 | 1.51 | 265 | 0.2186 |
| 10 | 1.40 | 1.45 | 97 | 0.2706 |

Working set:
#bin | resolution range | #ref1 |

| | | | | |
|---|---|---|---|---|
| 1 | 3.02 | 500.01 | 20201 | 0.1858 |
| 2 | 2.39 | 3.02 | 19732 | 0.1989 |
| 3 | 2.09 | 2.39 | 19506 | 0.1935 |
| 4 | 1.90 | 2.09 | 19199 | 0.1922 |
| 5 | 1.76 | 1.90 | 18521 | 0.1934 |
| 6 | 1.66 | 1.76 | 15253 | 0.1973 |
| 7 | 1.58 | 1.66 | 11380 | 0.1989 |
| 8 | 1.51 | 1.58 | 8211 | 0.2067 |
| 9 | 1.45 | 1.51 | 5065 | 0.2248 |
| 10 | 1.40 | 1.45 | 1999 | 0.2589 |

=============================== sigmaa-values
================================
sigmaa calculated using cross-validated data (test set)
number of bins for sigmaa calculation= 50

#bin | resolution range | #ref1 |

| | | | | |
|---|---|---|---|---|
| 1 | 5.16 | 500.01 | 4426 | 0.9319 |
| 2 | 4.09 | 5.16 | 4270 | 0.9576 |
| 3 | 3.58 | 4.09 | 4227 | 0.9615 |
| 4 | 3.25 | 3.58 | 4196 | 0.9632 |
| 5 | 3.02 | 3.25 | 4195 | 0.9422 |
| 6 | 2.84 | 3.02 | 4177 | 0.9512 |
| 7 | 2.70 | 2.84 | 4163 | 0.9369 |
| 8 | 2.58 | 2.70 | 4152 | 0.9424 |
| 9 | 2.48 | 2.58 | 4156 | 0.9486 |
| 10 | 2.39 | 2.48 | 4126 | 0.9448 |
| 11 | 2.32 | 2.39 | 4131 | 0.9407 |
| 12 | 2.25 | 2.32 | 4149 | 0.9498 |
| 13 | 2.19 | 2.25 | 4101 | 0.9417 |
| 14 | 2.14 | 2.19 | 4113 | 0.9351 |
| 15 | 2.09 | 2.14 | 4060 | 0.9459 |
| 16 | 2.05 | 2.09 | 4096 | 0.9333 |
| 17 | 2.01 | 2.05 | 4078 | 0.9552 |
| 18 | 1.97 | 2.01 | 4005 | 0.9445 |
| 19 | 1.93 | 1.97 | 4001 | 0.9443 |
| 20 | 1.90 | 1.93 | 4019 | 0.9352 |
| 21 | 1.87 | 1.90 | 3971 | 0.9411 |
| 22 | 1.84 | 1.87 | 3941 | 0.9319 |
| 23 | 1.81 | 1.84 | 3946 | 0.9360 |
| 24 | 1.79 | 1.81 | 3860 | 0.9480 |
| 25 | 1.76 | 1.79 | 3792 | 0.9213 |
| 26 | 1.74 | 1.76 | 3632 | 0.9184 |
| 27 | 1.72 | 1.74 | 3458 | 0.9382 |
| 28 | 1.70 | 1.72 | 3191 | 0.9414 |
| 29 | 1.68 | 1.70 | 2994 | 0.9275 |
| 30 | 1.66 | 1.68 | 2800 | 0.9268 |
| 31 | 1.64 | 1.66 | 2678 | 0.9303 |
| 32 | 1.62 | 1.64 | 2506 | 0.9388 |
| 33 | 1.61 | 1.62 | 2366 | 0.9506 |
| 34 | 1.59 | 1.61 | 2249 | 0.9402 |
| 35 | 1.58 | 1.59 | 2165 | 0.9270 |
| 36 | 1.56 | 1.58 | 2006 | 0.9365 |
| 37 | 1.55 | 1.56 | 1874 | 0.9184 |
| 38 | 1.53 | 1.55 | 1700 | 0.9072 |
| 39 | 1.52 | 1.53 | 1595 | 0.9358 |
| 40 | 1.51 | 1.52 | 1455 | 0.9398 |
| 41 | 1.50 | 1.51 | 1336 | 0.9433 |
| 42 | 1.48 | 1.50 | 1147 | 0.9541 |
| 43 | 1.47 | 1.48 | 1077 | 0.9419 |
| 44 | 1.46 | 1.47 | 962 | 0.9331 |
| 45 | 1.45 | 1.46 | 808 | 0.9242 |
| 46 | 1.44 | 1.45 | 696 | 0.9038 |
| 47 | 1.43 | 1.44 | 570 | 0.9371 |
| 48 | 1.42 | 1.43 | 408 | 0.9387 |
| 49 | 1.41 | 1.42 | 288 | 0.8914 |
| 50 | 1.40 | 1.41 | 134 | 0.8194 |

============================ non-trans peptides
==============================

| cis-peptide: | segid=A resid=406 resname=THR | |
|---|---|---|
| | current dihedral value= | −0.010 |
| cis-peptide: | segid=A resid=532 resname=PRO | |
| | current dihedral va1ue= | −0.435 |

=============================== occupancies
==================================
no atoms have zero occupancy

TABLE 6
Crystallographic Refinement Statistics for the Drosophila Golgi Mannosidase
II Associated with Swainsonine

===========================================================================
===
>>> input coordinates: swainsonine3_ann_1bi.pdb
>>> molecular structure file: swainsoninegen3.mtf
>>> parameter file 1 : CNS_TOPPAR:protein_rep.param
>>> parameter file 2 : CNS_TOPPAR:water_rep.param
>>> parameter file 3 : CNS_TOPPAR:ion.param
>>> parameter file 4 : swa.par
>>> parameter file 5 : ../zntrmp/mpdnew2.par
>>> parameter file 6 : cis_peptide.param
>>> parameter file 7 : CNS_TOPPAR:carbohydrate.param
>>> reflection file= dgm2native_rejmerge.cv
>>> spacegroup: P2 (1) 2 (1) 2 (1)
>>> cell dimensions: a= 68.902 b= 110.015 c= 138.472 alpha= 90 beta= 90
gamma= 90
>>> current wa= 0.632464 for target= mlf
>>> ncs= none
>>> initial B-factor correction applied to fobs :
>>> B11= 0.551 B22= −0.116 B33= 0.435
>>> B12= 0.000 B13= 0.000 B23= 0.000
>>> B-factor correction applied to coordinate array B: 0.413
>>> B-correction resolution: 6.0 - 1.87
>>> bulk solvent: density level= 0.354131 e/A\hat{ }3, B-factor= 42.2797 A\hat{ }2
=================================== summary
==================================
resolution range: 500.0 - 1.87 A

| R-values: | |
|---|---|
| initial | r= 0.2078 free_r= 0.2371 |
| after B-factor and/or bulk solvent correction | r= 0.1810 free_r= 0.2090 |

Monitor for target “mlf” is R-value :

working set= 0.1810 test set= 0.2090

| | luzzati coordinate error | (5.0 - 1.87 A ): | 0.19 A |
|---|---|---|---|
| cross-validated luzzati coordinate error | | (5.0 - 1.87 A ): | 0.22 A |
| | sigmaa coordinate error | (5.0 - 1.87 A ): | 0.12 A |

| | | |
|---|---|---|
| cross-validated sigmaa coordinate error | (5.0 - 1.87 A ): | 0.14 A |

rmsd bonds= 0.005230 with 2 bond violations > 0.05
rmsd angles= 1.31525 with 34 angle violations > 8.0
rmsd dihedrals= 24.18605 with 4 angle violations > 60.0
rmsd improper= 0.78741 with 34 angle violations > 3.0
================================== B-factors
=================================
average B-factor= 19.441
minimum B-factor= 8.18268
maximum B-factor= 64.5527
B rmsd for bonded mainchain atoms= 0.699
B rmsd for bonded sidechain atoms= 1.141
B rmsd for angle mainchain atoms= 1.167
B rmsd for angle sidechain atoms= 1.747
current rweight= 0.18691688
================================ diffraction data
============================
reflections with |Fobs|/sigma_F < 0.0 rejected
reflections with |Fobs| > 10000 * rms (Fobs) rejected

| | |
|---|---|
| theoretical total number of ref1. in reso1. range: | 87643 ( 100.0 % ) |
| number of unobserved reflections (no entry or \|F\|=0): | 2814 ( 3.2 % ) |
| number of reflections rejected: | 0 ( 0.0 % ) |
| total number of reflections used: | 84829 ( 96.8 % ) |
| number of reflections in working set: | 80543 ( 91.9 % ) |
| number of reflections in test set: | 4286 ( 4.9 % ) |

=======> completeness

Test set (test = 1):
#bin | resolution range | #ref1 |

| | | | | |
|---|---|---|---|---|
| 1 | 4.03 | 500.01 | 452 | 0.0493 |
| 2 | 3.20 | 4.03 | 488 | 0.0550 |
| 3 | 2.79 | 3.20 | 398 | 0.0453 |
| 4 | 2.54 | 2.79 | 433 | 0.0496 |
| 5 | 2.36 | 2.54 | 460 | 0.0527 |
| 6 | 2.22 | 2.36 | 433 | 0.0497 |
| 7 | 2.11 | 2.22 | 417 | 0.0480 |
| 8 | 2.01 | 2.11 | 402 | 0.0464 |
| 9 | 1.94 | 2.01 | 406 | 0.0470 |
| 10 | 1.87 | 1.94 | 397 | 0.0457 |

Working set:
#bin | resolution range | #ref1 |

| | | | | |
|---|---|---|---|---|
| 1 | 4.03 | 500.01 | 8521 | 0.9298 |
| 2 | 3.20 | 4.03 | 8330 | 0.9385 |
| 3 | 2.79 | 3.20 | 8244 | 0.9391 |
| 4 | 2.54 | 2.79 | 8134 | 0.9315 |
| 5 | 2.36 | 2.54 | 8056 | 0.9234 |
| 6 | 2.22 | 2.36 | 8022 | 0.9215 |
| 7 | 2.11 | 2.22 | 7985 | 0.9196 |
| 8 | 2.01 | 2.11 | 7940 | 0.9158 |
| 9 | 1.94 | 2.01 | 7835 | 0.9077 |
| 10 | 1.87 | 1.94 | 7476 | 0.8615 |

================================= R-values
===================================
=======> R-values with |Fobs|/sigma cutoff= 0.0

Test set (test = 1):
#bin | resolution range | #ref1 |

| | | | | |
|---|---|---|---|---|
| 1 | 4.03 | 500.01 | 452 | 0.2028 |
| 2 | 3.20 | 4.03 | 488 | 0.1836 |
| 3 | 2.79 | 3.20 | 398 | 0.2059 |
| 4 | 2.54 | 2.79 | 433 | 0.2246 |
| 5 | 2.36 | 2.54 | 460 | 0.2203 |
| 6 | 2.22 | 2.36 | 433 | 0.2125 |
| 7 | 2.11 | 2.22 | 417 | 0.2304 |
| 8 | 2.01 | 2.11 | 402 | 0.2066 |
| 9 | 1.94 | 2.01 | 406 | 0.2154 |
| 10 | 1.87 | 1.94 | 397 | 0.2693 |

Working set:
#bin | resolution range | #ref1 |

| | | | | |
|---|---|---|---|---|
| 1 | 4.03 | 500.01 | 8521 | 0.1673 |
| 2 | 3.20 | 4.03 | 8330 | 0.1704 |
| 3 | 2.79 | 3.20 | 8244 | 0.1861 |
| 4 | 2.54 | 2.79 | 8134 | 0.1857 |
| 5 | 2.36 | 2.54 | 8056 | 0.1830 |
| 6 | 2.22 | 2.36 | 8022 | 0.1827 |
| 7 | 2.11 | 2.22 | 7985 | 0.1894 |
| 8 | 2.01 | 2.11 | 7940 | 0.1826 |
| 9 | 1.94 | 2.01 | 7835 | 0.1920 |
| 10 | 1.87 | 1.94 | 7476 | 0.2320 |

=============================== sigmaa-values
================================
sigmaa calculated using cross-validated data (test set)
number of bins for sigmaa calculation= 50

#bin | resolution range | #ref1 |

| | | | | |
|---|---|---|---|---|
| 1 | 6.89 | 500.01 | 1778 | 0.9682 |
| 2 | 5.47 | 6.89 | 1834 | 0.9324 |
| 3 | 4.78 | 5.47 | 1793 | 0.9330 |
| 4 | 4.34 | 4.78 | 1797 | 0.9551 |
| 5 | 4.03 | 4.34 | 1771 | 0.9634 |
| 6 | 3.79 | 4.03 | 1778 | 0.9700 |
| 7 | 3.60 | 3.79 | 1751 | 0.9563 |
| 8 | 3.44 | 3.60 | 1749 | 0.9651 |
| 9 | 3.31 | 3.44 | 1775 | 0.9515 |
| 10 | 3.20 | 3.31 | 1765 | 0.9589 |
| 11 | 3.10 | 3.20 | 1714 | 0.9433 |
| 12 | 3.01 | 3.10 | 1746 | 0.9571 |
| 13 | 2.93 | 3.01 | 1737 | 0.9545 |
| 14 | 2.86 | 2.93 | 1721 | 0.9457 |
| 15 | 2.79 | 2.86 | 1724 | 0.9391 |
| 16 | 2.73 | 2.79 | 1727 | 0.9327 |
| 17 | 2.68 | 2.73 | 1708 | 0.9429 |
| 18 | 2.63 | 2.68 | 1726 | 0.9267 |
| 19 | 2.58 | 2.63 | 1737 | 0.9416 |
| 20 | 2.54 | 2.58 | 1669 | 0.9385 |
| 21 | 2.50 | 2.54 | 1705 | 0.9257 |
| 22 | 2.46 | 2.50 | 1713 | 0.9444 |
| 23 | 2.42 | 2.46 | 1721 | 0.9384 |
| 24 | 2.39 | 2.42 | 1665 | 0.9456 |
| 25 | 2.36 | 2.39 | 1712 | 0.9398 |
| 26 | 2.33 | 2.36 | 1699 | 0.9427 |
| 27 | 2.30 | 2.33 | 1675 | 0.9341 |
| 28 | 2.27 | 2.30 | 1723 | 0.9324 |
| 29 | 2.24 | 2.27 | 1653 | 0.9570 |
| 30 | 2.22 | 2.24 | 1705 | 0.9355 |
| 31 | 2.19 | 2.22 | 1711 | 0.9093 |
| 32 | 2.17 | 2.19 | 1650 | 0.9188 |
| 33 | 2.15 | 2.17 | 1659 | 0.9341 |
| 34 | 2.13 | 2.15 | 1707 | 0.9520 |
| 35 | 2.11 | 2.13 | 1675 | 0.9298 |
| 36 | 2.09 | 2.11 | 1655 | 0.9446 |
| 37 | 2.07 | 2.09 | 1691 | 0.9407 |
| 38 | 2.05 | 2.07 | 1685 | 0.9234 |
| 39 | 2.03 | 2.05 | 1631 | 0.9412 |
| 40 | 2.01 | 2.03 | 1680 | 0.9613 |
| 41 | 2.00 | 2.01 | 1646 | 0.9423 |
| 42 | 1.98 | 2.00 | 1669 | 0.9395 |
| 43 | 1.97 | 1.98 | 1626 | 0.9240 |
| 44 | 1.95 | 1.97 | 1693 | 0.9373 |
| 45 | 1.94 | 1.95 | 1607 | 0.9534 |
| 46 | 1.92 | 1.94 | 1634 | 0.9255 |
| 47 | 1.91 | 1.92 | 1652 | 0.9181 |
| 48 | 1.90 | 1.91 | 1616 | 0.9194 |
| 49 | 1.88 | 1.90 | 1597 | 0.9026 |
| 50 | 1.87 | 1.88 | 1374 | 0.8987 |

============================ non-trans peptides
==============================

| cis-peptide: | segid=A resid=406 resname=THR | |
|---|---|---|
| | current dihedral value= | 0.298 |
| cis-peptide: | segid=A resid=532 resname=PRO | |
| | current dihedral value= | −0.154 |

=============================== occupancies
==================================
no atoms have zero occupancy

TABLE 7
• Glycoside Hydrolase Classification

| | |
|---|---|
| CAZy Family | Glycoside Hydrolase Family 38 |
| Known Activities | α-mannosidase (EC 3.2.1.24) (EC 3.2.1.114). |
| Mechanism | Retaining |
| Catalytic | Asp |
| Nucleophile/Base | |
| Catalytic Proton Donor | Not known |
| 3D Structure Status | Not available |
| Relevant Links | InterPro; PFAM |
| Statistics | CAZy(48); GenBank/GenPept (108); Swissprot (31) |

| | | | | | |
|---|---|---|---|---|---|
| | | | | | PDB/ |
| Protein | Organism | EC# | GenBank/GenPept | SwissProt | 3D |
| α-mannosidase (MLJ15.11) | Arabidopsis thaliana | n.d. | AB026648 BAB01735.1 | P94078 | |
| | | | X98130 CAA66821.1 | Q96239 | |
| | | | Y11767 CAA72432.1 | | |
| QRF F2G14_70 | Arabidopsis thaliana | n.d. | AL391146 CAC01814.1 | Q9LFR0 | |
| | | | T51440 | | |
| ORF K2A18.23 | Arabidopsis thaliana | n.d | AB011474 BAB10420.1 | | |
| ORF MAC12.5 | Arabidopsis thaliana | n.d | AB005230 BAB11126.1 | | |
| α-mannosidase | Aspergillus nidulans | n.d | AF016850 AAB70514.1 | O13344 | |
| α-mannosidase (lysosomal) | Bos taurus | 3.2.1.24 | L31373 AAB67726.1 | Q29451 | |
| | | | U97686 AAC48763.1 | O02848 | |
| | | | U97687 AAC48763.1 | O19138 | |
| | | | U97688 AAC48763.1 | | |
| | | | U97689 AAC48763.1 | | |
| | | | U97690 AAC48769.1 | | |
| | | | U97691 AAC48763.1 | | |
| | | | U97692 AAC48763.1 | | |
| | | | U97693 AAC48763.1 | | |
| | | | U97694 AAC48763.1 | | |
| ORF F48C1.1 | Caenorhabditis elegans | n.d. | U97015 AAB52345.1 | O01574 | |
| ORF F55D10.1 | Caenorhabditis elegans | n.d. | U40948 AAA81731.1 | Q20829 | |
| ORF F58H1.1 | Caenorhabditis elegans | n.d. | Z75954 CAB00104.1 | Q21010 | |
| α-mannosidase | Canavalia ensiformis | 3.2.1.24 | | | |
| α-mannosidase | | 3.2.1.114 | M82822 AAA33224.1 | P34098 | |
| α-mannosidase (α-Man-IIb) | Drosophila melanogaster | 3.2.1.114 | AE003710 AAF55228.1 | | |
| | | | AB018079 BAA75817.1 | | |
| α-mannosidase 2 (α-Man-II) | Drosophila melanogaster | 3.2.1.114 | AE003682 AAF54375.1 | Q24451 | |
| (CG18474) | | | AE003682 AAF54376.1 | | |
| | | | AJ132715 CAA10755.1 | | |
| | | | X77652 CAA54732.1 | | |
| ORF BcDNA.GH02419 | Drosophila melanogaster | n.d. | AF145601 AAD38576.1 | | |
| | | | AE003628 AAF52958.1 | | |
| ORF CG5322 | Drosophila melanogaster | n.d. | AE003628 AAF52957.1 | | |
| ORF CG9463 | Drosophila melanogaster | n.d. | AE003622 AAF52708.1 | | |
| ORF CG9465 | Drosophila melanogaster | n.d. | AE003622 AAF52709.1 | | |
| ORF CG9466 | Drosophila melanogaster | n.d. | AE003622 AAF52710.1 | | |
| ORF CG9468 | Drosophila melanogaster | n.d. | AE003622 AAF52711.1 | | |
| ORF YbgB | Escherichia coli K12 | n.d. | D90713 BAA35398.1 | | |
| ORF YbgG | Escherichia coli K12/MG1655 | n.d. | AE000176 AAC73826.1 | P54746 | |
| | | | | P75753 | |
| α-mannosidase (lysosomal) | Felis catus | 3.2.1.24 | AF010191 AAB97672.1 | Q46432 | |
| | | | AF010192 AAB97733.1 | | |
| α-mannosidase | Homo sapiens | 3.2.1.114 | U31520 AAC50302.1 | Q16706 | |
| | | | D63998 BAA10017.1 | Q16767 | |
| α-mannosidase | Homo sapiens | n.d. | U37248 AAC00568.1 | Q13358 | |
| α-mannosidase (lysosomal) | Homo sapiens | 3.2.1.24 | U05572 AAB03816.1 | O00754 | |
| | | | U60266 AAC34130.1 | Q93094 | |
| | | | U68587 AAC50812.1 | Q16680 | |
| | | | U60885 AAC51362.1 | O15330 | |
| | | | U60886 AAC51362.1 | | |
| | | | U60887 AAC51362.1 | | |
| | | | U60888 AAC51362.1 | | |
| | | | U60889 AAC51362.1 | | |
| | | | U60890 AAC51362.1 | | |
| | | | U60891 AAC51362.1 | | |
| | | | U60892 AAC51362.1 | | |
| | | | U60893 AAC51362.1 | | |
| | | | U60894 AAC51362.1 | | |
| | | | U60895 AAC51362.1 | | |
| | | | U60896 AAC51362.1 | | |
| | | | U60897 AAC51362.1 | | |
| | | | U60898 AAC51362.1 | | |
| | | | U60899 AAC51362.1 | | |
| α-mannosidase (lysosomal) | Homo sapiens | 3.2.1.24 | U68382 AAC50811.1 | Q93093 | |
| α-mannosidase 2 | Homo sapiens | 3.2.1.114 | L28821 AAA92022.1 | P49641 | |
| | | | D55649 BAA09510.1 | Q13754 | |
| α-mannosidase 6A8B | Homo sapiens | n.d. | AF044414 AAC00190.1 | | |
| ORF DKFZp434D175 | Homo spaiens | n.d. | AL136876 CAB66810.1 | | |
| | | | T46931 | | |
| ORF KIAA0935 | Homo sapiens | n.d. | AB023152 BAA76779.1 | | |
| α-mannosidase | Mus musculus | 3.2.1.24 | AB006458 BAA24266.1 | O54782 | |
| α-mannosidase (lysosomal) | Mus musculus | 3.2.1.24 | U87240 AAC09470.1 | O09159 | |
| | | | U29947 AAC53369.1 | Q64443 | |
| α-mannosidase (lysosomal) | Mus musculus | 3.2.1.24 | NM_010764 6754622 | O55037 | |
| | | | AF044174 AAC78560.1 | | |
| | | | AF044175 AAC78560.1 | | |
| | | | AF044176 AAC78560.1 | | |
| | | | AF044177 AAC78560.1 | | |
| | | | AF044178 AAC78560.1 | | |
| | | | AF044179 AAC78560.1 | | |
| | | | AF044180 AAC78560.1 | | |
| | | | AF044181 AAC78560.1 | | |
| | | | AF044182 AAC78560.1 | | |
| | | | AF044183 AAC78560.1 | | |
| | | | AF044184 AAC78560.1 | | |
| | | | AF044185 AAC78560.1 | | |
| | | | AF044186 AAC78560.1 | | |
| | | | AF044187 AAC78560.1 | | |
| | | | AF044188 AAC78560.1 | | |
| | | | AF044189 AAC78560.1 | | |
| | | | AF044190 AAC78560.1 | | |
| | | | AF044191 AAC78560.1 | | |
| | | | AF044192 AAC78560.1 | | |
| α-mannosidase 2 | Mus musculus | 3.2.1.114 | X61172 CAA43480.1 | P27046 | |
| α-mannosidase IIx | Mus musculus | n.d. | AF107018 AAD20813.1 | | |
| ORF Rv0648 | Mycobacterium tuberculosis | n.d. | Z92772 CAB07105.1 | P96937 | |
| | H37Rv | | | | |
| ORF PH0835 | Pyrococcus horikoshii | n.d. | AP000003 BAA29929.1 | O5856 | |
| α-mannosidase 1 | Rattus norvegicus | 3.2.1.24 | M57547 AAA41565.1 | P21139 | |
| α-mannosidase 2 | Rattus norvegicus | 3.2.1.114 | M24353 AAA66457.1 | P28494 | |
| α-mannosidase | Saccharomyces cerevisiae | 3.2.1.24 | M27809 AAA34423.1 | P22855 | |
| | | | M29146 AAA34423.1 | | |
| | | | Z48618 CAA88536.1 | | |
| | | | Z72678 CAA96868.1 | | |
| ORF SPAC513.05 | Schizosaccharomyces pombe | n.d. | AL122032 CAB58728.1 | | |
| α-mannosidase II | Spodoptera frugiperda | n.d. | AF005034 AAB62719.1 | O18497 | |
| ORF SCM11.03c | Streptomyces coelicolor A3(2) | n.d. | AL133278 CAB61914.1 | | |
| α-mannosidase | Sus scrofa | 3.2.1.24 | D28521 BAA05877.1 | Q28949 | |
| ORF slr0323 (Ams1) | Synechocyslis sp. | n.d. | D63999 BAA10023.1 | Q55528 | |
| ORF TM1231 | Themotoga maritima | n.d. | AE001779 AAD36306.1 | Q9X0V8 | |
| ORF TM1851 | Themotoga maritima | n.d. | AE001822 AAD36913.1 | Q9X2G6 | |
| α-mannosidase 1 | Zea mays | 3.2.1.- | D30744 BAA06405.1 | Q43249 | |

TABLE 8

| | | | | | | | | | | |
|---|---|---|---|---|---|---|---|---|---|---|
| ATOM | 1 | N | CYS | A 31 | 44.192 | 36.201 | −18.860 | 1.00 | 0.00 | N |
| ATOM | 2 | CA | CYS | A 31 | 43.432 | 37.453 | −18.569 | 1.00 | 0.00 | C |
| ATOM | 3 | C | CYS | A 31 | 41.925 | 37.255 | −18.672 | 1.00 | 0.00 | C |
| ATOM | 4 | O | CYS | A 31 | 41.437 | 36.646 | −19.622 | 1.00 | 0.00 | O |
| ATOM | 5 | CB | CYS | A 31 | 43.821 | 38.563 | −19.546 | 1.00 | 0.00 | C |
| ATOM | 6 | SG | CYS | A 31 | 45.504 | 39.239 | −19.408 | 1.00 | 0.00 | S |
| ATOM | 7 | N | GLN | A 32 | 41.194 | 37.780 | −17.693 | 1.00 | 0.00 | N |
| ATOM | 8 | CA | GLN | A 32 | 39.739 | 37.692 | −17.702 | 1.00 | 0.00 | C |
| ATOM | 9 | C | GLN | A 32 | 39.199 | 38.594 | −18.800 | 1.00 | 0.00 | C |
| ATOM | 10 | O | GLN | A 32 | 39.785 | 39.630 | −19.113 | 1.00 | 0.00 | O |
| ATOM | 11 | CB | GLN | A 32 | 39.146 | 38.164 | −16.373 | 1.00 | 0.00 | C |
| ATOM | 12 | CG | GLN | A 32 | 39.160 | 37.145 | −15.258 | 1.00 | 0.00 | C |
| ATOM | 13 | CD | GLN | A 32 | 38.246 | 37.549 | −14.118 | 1.00 | 0.00 | C |
| ATOM | 14 | OE1 | GLN | A 32 | 37.027 | 37.630 | −14.285 | 1.00 | 0.00 | O |
| ATOM | 15 | NE2 | GLN | A 32 | 38.829 | 37.814 | −12.955 | 1.00 | 0.00 | N |
| ATOM | 16 | N | ASP | A 33 | 38.079 | 38.196 | −19.384 | 1.00 | 0.00 | N |
| ATOM | 17 | CA | ASP | A 33 | 37.449 | 38.989 | −20.427 | 1.00 | 0.00 | C |
| ATOM | 18 | C | ASP | A 33 | 36.549 | 39.962 | −19.668 | 1.00 | 0.00 | C |
| ATOM | 19 | O | ASP | A 33 | 35.610 | 39.545 | −18.998 | 1.00 | 0.00 | O |
| ATOM | 20 | CB | ASP | A 33 | 36.638 | 38.070 | −21.344 | 1.00 | 0.00 | C |
| ATOM | 21 | CG | ASP | A 33 | 36.037 | 38.801 | −22.524 | 1.00 | 0.00 | C |
| ATOM | 22 | OD1 | ASP | A 33 | 35.788 | 38.143 | −23.557 | 1.00 | 0.00 | O |
| ATOM | 23 | OD2 | ASP | A 33 | 35.802 | 40.024 | −22.416 | 1.00 | 0.00 | O |
| ATOM | 24 | N | VAL | A 34 | 36.844 | 41.255 | −19.757 | 1.00 | 0.00 | N |
| ATOM | 25 | CA | VAL | A 34 | 36.065 | 42.254 | −19.031 | 1.00 | 0.00 | C |
| ATOM | 26 | C | VAL | A 34 | 34.893 | 42.824 | −19.820 | 1.00 | 0.00 | C |
| ATOM | 27 | O | VAL | A 34 | 34.211 | 43.735 | −19.354 | 1.00 | 0.00 | O |
| ATOM | 28 | CB | VAL | A 34 | 36.969 | 43.425 | −18.568 | 1.00 | 0.00 | C |
| ATOM | 29 | CG1 | VAL | A 34 | 38.134 | 42.887 | −17.755 | 1.00 | 0.00 | C |
| ATOM | 30 | CG2 | VAL | A 34 | 37.491 | 44.203 | −19.774 | 1.00 | 0.00 | C |
| ATOM | 31 | N | VAL | A 35 | 34.639 | 42.272 | −21.001 | 1.00 | 0.00 | N |
| ATOM | 32 | CA | VAL | A 35 | 33.557 | 42.772 | −21.836 | 1.00 | 0.00 | C |
| ATOM | 33 | C | VAL | A 35 | 32.381 | 41.825 | −22.061 | 1.00 | 0.00 | C |
| ATOM | 34 | O | VAL | A 35 | 31.224 | 42.211 | −21.898 | 1.00 | 0.00 | O |
| ATOM | 35 | CB | VAL | A 35 | 34.092 | 43.170 | −23.236 | 1.00 | 0.00 | C |
| ATOM | 36 | CG1 | VAL | A 35 | 32.946 | 43.675 | −24.118 | 1.00 | 0.00 | C |
| ATOM | 37 | CG2 | VAL | A 35 | 35.181 | 44.222 | −23.098 | 1.00 | 0.00 | C |
| ATOM | 38 | N | GLN | A 36 | 32.688 | 40.584 | −22.417 | 1.00 | 0.00 | N |
| ATOM | 39 | CA | GLN | A 36 | 31.668 | 39.594 | −22.768 | 1.00 | 0.00 | C |
| ATOM | 40 | C | GLN | A 36 | 31.067 | 38.670 | −21.719 | 1.00 | 0.00 | C |
| ATOM | 41 | O | GLN | A 36 | 30.128 | 37.936 | −22.022 | 1.00 | 0.00 | O |
| ATOM | 42 | CB | GLN | A 36 | 32.208 | 38.745 | −23.915 | 1.00 | 0.00 | C |
| ATOM | 43 | CG | GLN | A 36 | 32.967 | 39.567 | −24.944 | 1.00 | 0.00 | C |
| ATOM | 44 | CD | GLN | A 36 | 33.436 | 38.744 | −26.118 | 1.00 | 0.00 | C |
| ATOM | 45 | OE1 | GLN | A 36 | 32.665 | 38.450 | −27.029 | 1.00 | 0.00 | O |
| ATOM | 46 | NE2 | GLN | A 36 | 34.705 | 38.357 | −26.100 | 1.00 | 0.00 | N |
| ATOM | 47 | N | ASP | A 37 | 31.592 | 38.681 | −20.501 | 1.00 | 0.00 | N |
| ATOM | 48 | CA | ASP | A 37 | 31.055 | 37.814 | −19.457 | 1.00 | 0.00 | C |
| ATOM | 49 | C | ASP | A 37 | 30.419 | 38.615 | −18.328 | 1.00 | 0.00 | C |
| ATOM | 50 | O | ASP | A 37 | 31.123 | 39.192 | −17.501 | 1.00 | 0.00 | O |
| ATOM | 51 | CB | ASP | A 37 | 32.155 | 36.919 | −18.869 | 1.00 | 0.00 | C |
| ATOM | 52 | CG | ASP | A 37 | 32.746 | 35.967 | −19.890 | 1.00 | 0.00 | C |
| ATOM | 53 | OD1 | ASP | A 37 | 31.969 | 35.306 | −20.608 | 1.00 | 0.00 | O |
| ATOM | 54 | OD2 | ASP | A 37 | 33.989 | 35.872 | −19.964 | 1.00 | 0.00 | O |
| ATOM | 55 | N | VAL | A 38 | 29.091 | 38.641 | −18.294 | 1.00 | 0.00 | N |
| ATOM | 56 | CA | VAL | A 38 | 28.365 | 39.359 | −17.252 | 1.00 | 0.00 | C |
| ATOM | 57 | C | VAL | A 38 | 28.525 | 38.640 | −15.914 | 1.00 | 0.00 | C |
| ATOM | 58 | O | VAL | A 38 | 28.035 | 37.522 | −15.734 | 1.00 | 0.00 | O |
| ATOM | 59 | CB | VAL | A 38 | 26.863 | 39.454 | —17.582 | 1.00 | 0.00 | C |
| ATOM | 60 | CG1 | VAL | A 38 | 26.125 | 40.198 | −16.470 | 1.00 | 0.00 | C |
| ATOM | 61 | CG2 | VAL | A 38 | 26.672 | 40.164 | −18.920 | 1.00 | 0.00 | C |
| ATOM | 62 | N | PRO | A 39 | 29.218 | 39.272 | −14.957 | 1.00 | 0.00 | N |
| ATOM | 63 | CA | PRO | A 39 | 29.417 | 38.652 | −13.645 | 1.00 | 0.00 | C |
| ATOM | 64 | C | PRO | A 39 | 28.110 | 38.256 | −12.969 | 1.00 | 0.00 | C |
| ATOM | 65 | O | PRO | A 39 | 27.101 | 38.954 | −13.076 | 1.00 | 0.00 | O |
| ATOM | 66 | CB | PRO | A 39 | 30.157 | 39.735 | −12.863 | 1.00 | 0.00 | C |
| ATOM | 67 | CG | PRO | A 39 | 30.953 | 40.428 | −13.925 | 1.00 | 0.00 | C |
| ATOM | 68 | CD | PRO | A 39 | 29.934 | 40.558 | −15.041 | 1.00 | 0.00 | C |
| ATOM | 69 | N | ASN | A 40 | 28.126 | 37.122 | −12.278 | 1.00 | 0.00 | N |
| ATOM | 70 | CA | ASN | A 40 | 26.943 | 36.673 | −11.561 | 1.00 | 0.00 | C |
| ATOM | 71 | C | ASN | A 40 | 27.128 | 37.021 | −10.091 | 1.00 | 0.00 | C |
| ATOM | 72 | O | ASN | A 40 | 27.913 | 36.385 | −9.390 | 1.00 | 0.00 | O |
| ATOM | 73 | CB | ASN | A 40 | 26.752 | 35.164 | −11.707 | 1.00 | 0.00 | C |
| ATOM | 74 | CG | ASN | A 40 | 25.582 | 34.652 | −10.891 | 1.00 | 0.00 | C |
| ATOM | 75 | OD1 | ASN | A 40 | 24.465 | 35.162 | −10.999 | 1.00 | 0.00 | O |
| ATOM | 76 | ND2 | ASN | A 40 | 25.830 | 33.640 | −10.068 | 1.00 | 0.00 | N |
| ATOM | 77 | N | VAL | A 41 | 26.411 | 38.038 | −9.627 | 1.00 | 0.00 | N |
| ATOM | 78 | CA | VAL | A 41 | 26.518 | 38.457 | −8.233 | 1.00 | 0.00 | C |
| ATOM | 79 | C | VAL | A 41 | 25.157 | 38.452 | −7.549 | 1.00 | 0.00 | C |
| ATOM | 80 | O | VAL | A 41 | 24.120 | 38.549 | −8.204 | 1.00 | 0.00 | O |
| ATOM | 81 | CB | VAL | A 41 | 27.131 | 39.869 | −8.120 | 1.00 | 0.00 | C |
| ATOM | 82 | CG1 | VAL | A 41 | 28.573 | 39.851 | −8.604 | 1.00 | 0.00 | C |
| ATOM | 83 | CG2 | VAL | A 41 | 26.313 | 40.855 | −8.941 | 1.00 | 0.00 | C |
| ATOM | 84 | N | ASP | A 42 | 25.166 | 38.332 | −6.228 | 1.00 | 0.00 | N |
| ATOM | 85 | CA | ASP | A 42 | 23.928 | 38.315 | −5.462 | 1.00 | 0.00 | C |
| ATOM | 86 | C | ASP | A 42 | 23.258 | 39.679 | −5.488 | 1.00 | 0.00 | C |
| ATOM | 87 | O | ASP | A 42 | 22.032 | 39.782 | −5.528 | 1.00 | 0.00 | O |
| ATOM | 88 | CB | ASP | A 42 | 24.210 | 37.904 | −4.016 | 1.00 | 0.00 | C |
| ATOM | 89 | CG | ASP | A 42 | 24.747 | 36.489 | −3.909 | 1.00 | 0.00 | C |
| ATOM | 90 | OD1 | ASP | A 42 | 24.078 | 35.565 | −4.416 | 1.00 | 0.00 | O |
| ATOM | 91 | OD2 | ASP | A 42 | 25.830 | 36.300 | −3.319 | 1.00 | 0.00 | O |
| ATOM | 92 | N | VAL | A 43 | 24.072 | 40.729 | −5.455 | 1.00 | 0.00 | N |
| ATOM | 93 | CA | VAL | A 43 | 23.564 | 42.091 | −5.480 | 1.00 | 0.00 | C |
| ATOM | 94 | C | VAL | A 43 | 24.314 | 42.921 | −6.517 | 1.00 | 0.00 | C |
| ATOM | 95 | O | VAL | A 43 | 25.540 | 43.034 | −6.466 | 1.00 | 0.00 | O |
| ATOM | 96 | CB | VAL | A 43 | 23.729 | 42.785 | −4.111 | 1.00 | 0.00 | C |
| ATOM | 97 | CG1 | VAL | A 43 | 23.136 | 44.192 | −4.164 | 1.00 | 0.00 | C |
| ATOM | 98 | CG2 | VAL | A 43 | 23.063 | 41.963 | −3.017 | 1.00 | 0.00 | C |
| ATOM | 99 | N | GLN | A 44 | 23.576 | 43.479 | −7.468 | 1.00 | 0.00 | N |
| ATOM | 100 | CA | GLN | A 44 | 24.170 | 44.328 | −8.493 | 1.00 | 0.00 | C |
| ATOM | 101 | C | GLN | A 44 | 23.410 | 45.642 | −8.320 | 1.00 | 0.00 | C |
| ATOM | 102 | O | GLN | A 44 | 22.207 | 45.717 | −8.577 | 1.00 | 0.00 | O |
| ATOM | 103 | CB | GLN | A 44 | 23.956 | 43.723 | −9.884 | 1.00 | 0.00 | C |
| ATOM | 104 | CG | GLN | A 44 | 25.020 | 44.138 | −10.893 | 1.00 | 0.00 | C |
| ATOM | 105 | CD | GLN | A 44 | 25.127 | 45.648 | −11.011 | 1.00 | 0.00 | C |
| ATOM | 106 | OE1 | GLN | A 44 | 24.143 | 46.324 | −11.294 | 1.00 | 0.00 | O |
| ATOM | 107 | NE2 | GLN | A 44 | 26.325 | 46.181 | −10.790 | 1.00 | 0.00 | N |
| ATOM | 108 | N | MET | A 45 | 24.115 | 46.675 | −7.871 | 1.00 | 0.00 | N |
| ATOM | 109 | CA | MET | A 45 | 23.483 | 47.954 | −7.577 | 1.00 | 0.00 | C |
| ATOM | 110 | C | MET | A 45 | 22.592 | 48.605 | −8.632 | 1.00 | 0.00 | C |
| ATOM | 111 | O | MET | A 45 | 21.596 | 49.236 | −8.278 | 1.00 | 0.00 | O |
| ATOM | 112 | CB | MET | A 45 | 24.535 | 48.947 | −7.076 | 1.00 | 0.00 | C |
| ATOM | 113 | CG | MET | A 45 | 25.143 | 48.549 | −5.728 | 1.00 | 0.00 | C |
| ATOM | 114 | SD | MET | A 45 | 23.891 | 48.155 | −4.467 | 1.00 | 0.00 | S |
| ATOM | 115 | CE | MET | A 45 | 23.316 | 49.800 | −4.038 | 1.00 | 0.00 | C |
| ATOM | 116 | N | LEU | A 46 | 22.927 | 48.467 | −9.910 | 1.00 | 0.00 | N |
| ATOM | 117 | CA | LEU | A 46 | 22.087 | 49.063 | −10.949 | 1.00 | 0.00 | C |
| ATOM | 118 | C | LEU | A 46 | 20.770 | 48.286 | −11.009 | 1.00 | 0.00 | C |
| ATOM | 119 | O | LEU | A 46 | 19.689 | 48.870 | −11.150 | 1.00 | 0.00 | O |
| ATOM | 120 | CB | LEU | A 46 | 22.781 | 49.009 | −12.317 | 1.00 | 0.00 | C |
| ATOM | 121 | CG | LEU | A 46 | 22.024 | 49.702 | −13.459 | 1.00 | 0.00 | C |
| ATOM | 122 | CD1 | LEU | A 46 | 22.010 | 51.211 | −13.228 | 1.00 | 0.00 | C |
| ATOM | 123 | CD2 | LEU | A 46 | 22.686 | 49.379 | −14.791 | 1.00 | 0.00 | C |
| ATOM | 124 | N | GLU | A 47 | 20.868 | 46.963 | −10.890 | 1.00 | 0.00 | N |
| ATOM | 125 | CA | GLU | A 47 | 19.683 | 46.112 | −10.925 | 1.00 | 0.00 | C |
| ATOM | 126 | C | GLU | A 47 | 18.826 | 46.412 | −9.702 | 1.00 | 0.00 | C |
| ATOM | 127 | O | GLU | A 47 | 17.609 | 46.566 | −9.807 | 1.00 | 0.00 | O |
| ATOM | 128 | CB | GLU | A 47 | 20.086 | 44.632 | −10.946 | 1.00 | 0.00 | C |
| ATOM | 129 | CG | GLU | A 47 | 18.929 | 43.665 | −11.196 | 1.00 | 0.00 | C |
| ATOM | 130 | CD | GLU | A 47 | 18.116 | 43.357 | −9.952 | 1.00 | 0.00 | C |
| ATOM | 131 | OE1 | GLU | A 47 | 17.007 | 42.796 | −10.093 | 1.00 | 0.00 | O |
| ATOM | 132 | OE2 | GLU | A 47 | 18.582 | 43.657 | −8.832 | 1.00 | 0.00 | O |
| ATOM | 133 | N | LEU | A 48 | 19.467 | 46.514 | −8.542 | 1.00 | 0.00 | N |
| ATOM | 134 | CA | LEU | A 48 | 18.757 | 46.813 | −7.302 | 1.00 | 0.00 | C |
| ATOM | 135 | C | LEU | A 48 | 18.022 | 48.144 | −7.416 | 1.00 | 0.00 | C |
| ATOM | 136 | O | LEU | A 48 | 16.861 | 48.258 | −7.026 | 1.00 | 0.00 | O |
| ATOM | 137 | CB | LEU | A 48 | 19.737 | 46.869 | −6.125 | 1.00 | 0.00 | C |
| ATOM | 138 | CG | LEU | A 48 | 19.124 | 47.144 | −4.747 | 1.00 | 0.00 | C |
| ATOM | 139 | CD1 | LEU | A 48 | 18.094 | 46.072 | −4.423 | 1.00 | 0.00 | C |
| ATOM | 140 | CD2 | LEU | A 48 | 20.216 | 47.174 | −3.688 | 1.00 | 0.00 | C |
| ATOM | 141 | N | TYR | A 49 | 18.698 | 49.149 | −7.962 | 1.00 | 0.00 | N |
| ATOM | 142 | CA | TYR | A 49 | 18.097 | 50.470 | −8.120 | 1.00 | 0.00 | C |
| ATOM | 143 | C | TYR | A 49 | 16.855 | 50.421 | −9.002 | 1.00 | 0.00 | C |
| ATOM | 144 | O | TYR | A 49 | 15.883 | 51.141 | −8.771 | 1.00 | 0.00 | O |
| ATOM | 145 | CB | TYR | A 49 | 19.126 | 51.449 | −8.704 | 1.00 | 0.00 | C |
| ATOM | 146 | CG | TYR | A 49 | 19.677 | 52.405 | −7.670 | 1.00 | 0.00 | C |
| ATOM | 147 | CD1 | TYR | A 49 | 20.153 | 51.934 | −6.444 | 1.00 | 0.00 | C |
| ATOM | 148 | CD2 | TYR | A 49 | 19.672 | 53.783 | −7.892 | 1.00 | 0.00 | C |
| ATOM | 149 | CE1 | TYR | A 49 | 20.602 | 52.812 | −5.458 | 1.00 | 0.00 | C |
| ATOM | 150 | CE2 | TYR | A 49 | 20.119 | 54.671 | −6.913 | 1.00 | 0.00 | C |
| ATOM | 151 | CZ | TYR | A 49 | 20.578 | 54.180 | −5.700 | 1.00 | 0.00 | C |
| ATOM | 152 | OH | TYR | A 49 | 20.977 | 55.057 | −4.717 | 1.00 | 0.00 | O |
| ATOM | 153 | N | ASP | A 50 | 16.888 | 49.553 | −10.005 | 1.00 | 0.00 | N |
| ATOM | 154 | CA | ASP | A 50 | 15.768 | 49.407 | −10.925 | 1.00 | 0.00 | C |
| ATOM | 155 | C | ASP | A 50 | 14.532 | 48.872 | −10.192 | 1.00 | 0.00 | C |
| ATOM | 156 | O | ASP | A 50 | 13.409 | 49.299 | −10.465 | 1.00 | 0.00 | O |
| ATOM | 157 | CB | ASP | A 50 | 16.158 | 48.452 | −12.058 | 1.00 | 0.00 | C |
| ATOM | 158 | CG | ASP | A 50 | 15.327 | 48.653 | −13.311 | 1.00 | 0.00 | C |
| ATOM | 159 | OD1 | ASP | A 50 | 15.409 | 47.790 | −14.212 | 1.00 | 0.00 | O |
| ATOM | 160 | OD2 | ASP | A 50 | 14.609 | 49.672 | −13.403 | 1.00 | 0.00 | O |
| ATOM | 161 | N | ARG | A 51 | 14.749 | 47.951 | −9.256 | 1.00 | 0.00 | N |
| ATOM | 162 | CA | ARG | A 51 | 13.660 | 47.333 | −8.495 | 1.00 | 0.00 | C |
| ATOM | 163 | C | ARG | A 51 | 13.163 | 48.089 | −7.262 | 1.00 | 0.00 | C |
| ATOM | 164 | O | ARG | A 51 | 11.986 | 48.002 | −6.920 | 1.00 | 0.00 | O |
| ATOM | 165 | CB | ARG | A 51 | 14.057 | 45.914 | −8.062 | 1.00 | 0.00 | C |
| ATOM | 166 | CG | ARG | A 51 | 14.171 | 44.923 | −9.198 | 1.00 | 0.00 | C |
| ATOM | 167 | CD | ARG | A 51 | 14.347 | 43.487 | −8.698 | 1.00 | 0.00 | C |
| ATOM | 168 | NE | ARG | A 51 | 15.636 | 43.257 | −8.047 | 1.00 | 0.00 | N |
| ATOM | 169 | CZ | ARG | A 51 | 15.881 | 43.459 | −6.756 | 1.00 | 0.00 | C |
| ATOM | 170 | NH1 | ARG | A 51 | 14.923 | 43.899 | −5.951 | 1.00 | 0.00 | N |
| ATOM | 171 | NH2 | ARG | A 51 | 17.092 | 43.219 | −6.269 | 1.00 | 0.00 | N |
| ATOM | 172 | N | MET | A 52 | 14.050 | 48.818 | −6.592 | 1.00 | 0.00 | N |
| ATOM | 173 | CA | MET | A 52 | 13.680 | 49.560 | −5.385 | 1.00 | 0.00 | C |
| ATOM | 174 | C | MET | A 52 | 12.640 | 50.646 | −5.618 | 1.00 | 0.00 | C |
| ATOM | 175 | O | MET | A 52 | 12.600 | 51.260 | −6.684 | 1.00 | 0.00 | O |
| ATOM | 176 | CB | MET | A 52 | 14.923 | 50.187 | −4.754 | 1.00 | 0.00 | C |
| ATOM | 177 | CG | MET | A 52 | 15.887 | 49.188 | −4.155 | 1.00 | 0.00 | C |
| ATOM | 178 | SD | MET | A 52 | 17.401 | 50.006 | −3.610 | 1.00 | 0.00 | S |
| ATOM | 179 | CE | MET | A 52 | 16.765 | 51.029 | −2.290 | 1.00 | 0.00 | C |
| ATOM | 180 | N | SER | A 53 | 11.811 | 50.891 | −4.605 | 1.00 | 0.00 | N |
| ATOM | 181 | CA | SER | A 53 | 10.762 | 51.903 | −4.696 | 1.00 | 0.00 | C |
| ATOM | 182 | C | SER | A 53 | 11.154 | 53.233 | −4.054 | 1.00 | 0.00 | C |
| ATOM | 183 | O | SER | A 53 | 10.522 | 54.260 | −4.301 | 1.00 | 0.00 | O |
| ATOM | 184 | CB | SER | A 53 | 9.476 | 51.380 | −4.049 | 1.00 | 0.00 | C |
| ATOM | 185 | OG | SER | A 53 | 8.986 | 50.248 | −4.749 | 1.00 | 0.00 | O |
| ATOM | 186 | N | PHE | A 54 | 12.189 | 53.202 | −3.220 | 1.00 | 0.00 | N |
| ATOM | 187 | CA | PHE | A 54 | 12.689 | 54.396 | −2.542 | 1.00 | 0.00 | C |
| ATOM | 188 | C | PHE | A 54 | 11.648 | 55.183 | −1.749 | 1.00 | 0.00 | C |
| ATOM | 189 | O | PHE | A 54 | 11.735 | 56.409 | −1.656 | 1.00 | 0.00 | O |
| ATOM | 190 | CB | PHE | A 54 | 13.356 | 55.342 | −3.549 | 1.00 | 0.00 | C |
| ATOM | 191 | CG | PHE | A 54 | 14.604 | 54.786 | −4.178 | 1.00 | 0.00 | C |
| ATOM | 192 | CD1 | PHE | A 54 | 14.528 | 53.915 | −5.262 | 1.00 | 0.00 | C |
| ATOM | 193 | CD2 | PHE | A 54 | 15.855 | 55.131 | −3.681 | 1.00 | 0.00 | C |
| ATOM | 194 | CE1 | PHE | A 54 | 15.682 | 53.394 | −5.843 | 1.00 | 0.00 | C |
| ATOM | 195 | CE2 | PHE | A 54 | 17.019 | 54.617 | −4.250 | 1.00 | 0.00 | C |
| ATOM | 196 | CZ | PHE | A 54 | 16.935 | 53.746 | −5.334 | 1.00 | 0.00 | C |
| ATOM | 197 | N | LYS | A 55 | 10.668 | 54.498 | −1.170 | 1.00 | 0.00 | N |
| ATOM | 198 | CA | LYS | A 55 | 9.657 | 55.203 | −0.390 | 1.00 | 0.00 | C |
| ATOM | 199 | C | LYS | A 55 | 10.267 | 55.646 | 0.934 | 1.00 | 0.00 | C |
| ATOM | 200 | O | LYS | A 55 | 10.922 | 54.863 | 1.617 | 1.00 | 0.00 | O |
| ATOM | 201 | CB | LYS | A 55 | 8.442 | 54.305 | −0.139 | 1.00 | 0.00 | C |
| ATOM | 202 | CG | LYS | A 55 | 7.755 | 53.841 | −1.410 | 1.00 | 0.00 | C |
| ATOM | 203 | CD | LYS | A 55 | 7.334 | 55.014 | −2.285 | 1.00 | 0.00 | C |
| ATOM | 204 | CE | LYS | A 55 | 6.694 | 54.526 | −3.578 | 1.00 | 0.00 | C |
| ATOM | 205 | NZ | LYS | A 55 | 6.200 | 55.652 | −4.420 | 1.00 | 0.00 | N |
| ATOM | 206 | N | ASP | A 56 | 10.048 | 56.907 | 1.286 | 1.00 | 0.00 | N |
| ATOM | 207 | CA | ASP | A 56 | 10.584 | 57.476 | 2.515 | 1.00 | 0.00 | C |
| ATOM | 208 | C | ASP | A 56 | 9.524 | 57.491 | 3.618 | 1.00 | 0.00 | C |
| ATOM | 209 | O | ASP | A 56 | 8.964 | 58.535 | 3.942 | 1.00 | 0.00 | O |
| ATOM | 210 | CB | ASP | A 56 | 11.093 | 58.895 | 2.224 | 1.00 | 0.00 | C |
| ATOM | 211 | CG | ASP | A 56 | 11.696 | 59.569 | 3.438 | 1.00 | 0.00 | C |
| ATOM | 212 | OD1 | ASP | A 56 | 12.224 | 58.863 | 4.320 | 1.00 | 0.00 | O |
| ATOM | 213 | OD2 | ASP | A 56 | 11.654 | 60.815 | 3.502 | 1.00 | 0.00 | O |
| ATOM | 214 | N | ILE | A 57 | 9.250 | 56.327 | 4.198 | 1.00 | 0.00 | N |
| ATOM | 215 | CA | ILE | A 57 | 8.247 | 56.245 | 5.252 | 1.00 | 0.00 | C |
| ATOM | 216 | C | ILE | A 57 | 8.873 | 56.247 | 6.640 | 1.00 | 0.00 | C |
| ATOM | 217 | O | ILE | A 57 | 10.031 | 55.868 | 6.814 | 1.00 | 0.00 | O |
| ATOM | 218 | CB | ILE | A 57 | 7.371 | 54.976 | 5.110 | 1.00 | 0.00 | C |
| ATOM | 219 | CG1 | ILE | A 57 | 8.178 | 53.733 | 5.479 | 1.00 | 0.00 | C |
| ATOM | 220 | CG2 | ILE | A 57 | 6.855 | 54.854 | 3.679 | 1.00 | 0.00 | C |
| ATOM | 221 | CD1 | ILE | A 57 | 7.348 | 52.469 | 5.538 | 1.00 | 0.00 | C |
| ATOM | 222 | N | ASP | A 58 | 8.091 | 56.683 | 7.622 | 1.00 | 0.00 | N |
| ATOM | 223 | CA | ASP | A 58 | 8.527 | 56.744 | 9.013 | 1.00 | 0.00 | C |
| ATOM | 224 | C | ASP | A 58 | 8.552 | 55.333 | 9.595 | 1.00 | 0.00 | C |
| ATOM | 225 | O | ASP | A 58 | 7.504 | 54.718 | 9.787 | 1.00 | 0.00 | O |
| ATOM | 226 | CB | ASP | A 58 | 7.562 | 57.627 | 9.809 | 1.00 | 0.00 | C |
| ATOM | 227 | CG | ASP | A 58 | 7.981 | 57.808 | 11.257 | 1.00 | 0.00 | C |
| ATOM | 228 | OD1 | ASP | A 58 | 7.386 | 58.671 | 11.938 | 1.00 | 0.00 | O |
| ATOM | 229 | OD2 | ASP | A 58 | 8.892 | 57.094 | 11.723 | 1.00 | 0.00 | O |
| ATOM | 230 | N | GLY | A 59 | 9.749 | 54.825 | 9.876 | 1.00 | 0.00 | N |
| ATOM | 231 | CA | GLY | A 59 | 9.871 | 53.482 | 10.418 | 1.00 | 0.00 | C |
| ATOM | 232 | C | GLY | A 59 | 9.835 | 53.393 | 11.933 | 1.00 | 0.00 | C |
| ATOM | 233 | O | GLY | A 59 | 10.035 | 52.318 | 12.497 | 1.00 | 0.00 | O |
| ATOM | 234 | N | GLY | A 60 | 9.576 | 54.516 | 12.595 | 1.00 | 0.00 | N |
| ATOM | 235 | CA | GLY | A 60 | 9.527 | 54.525 | 14.048 | 1.00 | 0.00 | C |
| ATOM | 236 | C | GLY | A 60 | 10.794 | 55.129 | 14.623 | 1.00 | 0.00 | C |
| ATOM | 237 | O | GLY | A 60 | 11.352 | 56.059 | 14.041 | 1.00 | 0.00 | O |
| ATOM | 238 | N | VAL | A 61 | 11.261 | 54.611 | 15.756 | 1.00 | 0.00 | N |
| ATOM | 239 | CA | VAL | A 61 | 12.481 | 55.143 | 16.353 | 1.00 | 0.00 | C |
| ATOM | 240 | C | VAL | A 61 | 13.608 | 55.074 | 15.323 | 1.00 | 0.00 | C |
| ATOM | 241 | O | VAL | A 61 | 14.432 | 55.985 | 15.240 | 1.00 | 0.00 | O |
| ATOM | 242 | CB | VAL | A 61 | 12.861 | 54.396 | 17.657 | 1.00 | 0.00 | C |
| ATOM | 243 | CG1 | VAL | A 61 | 11.816 | 54.694 | 18.731 | 1.00 | 0.00 | C |
| ATOM | 244 | CG2 | VAL | A 61 | 12.957 | 52.900 | 17.413 | 1.00 | 0.00 | C |
| ATOM | 245 | N | TRP | A 62 | 13.652 | 53.995 | 14.545 | 1.00 | 0.00 | N |
| ATOM | 246 | CA | TRP | A 62 | 14.643 | 53.904 | 13.477 | 1.00 | 0.00 | C |
| ATOM | 247 | C | TRP | A 62 | 13.855 | 54.516 | 12.326 | 1.00 | 0.00 | C |
| ATOM | 248 | O | TRP | A 62 | 13.198 | 53.816 | 11.552 | 1.00 | 0.00 | O |
| ATOM | 249 | CB | TRP | A 62 | 15.022 | 52.450 | 13.165 | 1.00 | 0.00 | C |
| ATOM | 250 | CG | TRP | A 62 | 15.979 | 52.328 | 11.998 | 1.00 | 0.00 | C |
| ATOM | 251 | CD1 | TRP | A 62 | 16.663 | 53.346 | 11.388 | 1.00 | 0.00 | C |
| ATOM | 252 | CD2 | TRP | A 62 | 16.332 | 51.131 | 11.290 | 1.00 | 0.00 | C |
| ATOM | 253 | NE1 | TRP | A 62 | 17.412 | 52.859 | 10.343 | 1.00 | 0.00 | N |
| ATOM | 254 | CE2 | TRP | A 62 | 17.230 | 51.503 | 10.260 | 1.00 | 0.00 | C |
| ATOM | 255 | CE3 | TRP | A 62 | 15.978 | 49.781 | 11.423 | 1.00 | 0.00 | C |
| ATOM | 256 | CZ2 | TRP | A 62 | 17.776 | 50.573 | 9.368 | 1.00 | 0.00 | C |
| ATOM | 257 | CZ3 | TRP | A 62 | 16.520 | 48.855 | 10.537 | 1.00 | 0.00 | C |
| ATOM | 258 | CH2 | TRP | A 62 | 17.412 | 49.257 | 9.520 | 1.00 | 0.00 | C |
| ATOM | 259 | N | LYS | A 63 | 13.909 | 55.841 | 12.245 | 1.00 | 0.00 | N |
| ATOM | 260 | CA | LYS | A 63 | 13.164 | 56.598 | 11.245 | 1.00 | 0.00 | C |
| ATOM | 261 | C | LYS | A 63 | 13.183 | 56.106 | 9.802 | 1.00 | 0.00 | C |
| ATOM | 262 | O | LYS | A 63 | 12.177 | 56.219 | 9.094 | 1.00 | 0.00 | O |
| ATOM | 263 | CB | LYS | A 63 | 13.595 | 58.067 | 11.290 | 1.00 | 0.00 | C |
| ATOM | 264 | CG | LYS | A 63 | 13.127 | 58.819 | 12.543 | 1.00 | 0.00 | C |
| ATOM | 265 | CD | LYS | A 63 | 11.605 | 58.967 | 12.572 | 1.00 | 0.00 | C |
| ATOM | 266 | CE | LYS | A 63 | 11.120 | 59.794 | 13.759 | 1.00 | 0.00 | C |
| ATOM | 267 | NZ | LYS | A 63 | 11.458 | 59.205 | 15.096 | 1.00 | 0.00 | N |
| ATOM | 268 | N | GLN | A 64 | 14.305 | 55.556 | 9.358 | 1.00 | 0.00 | N |
| ATOM | 269 | CA | GLN | A 64 | 14.401 | 55.101 | 7.976 | 1.00 | 0.00 | C |
| ATOM | 270 | C | GLN | A 64 | 14.460 | 53.584 | 7.816 | 1.00 | 0.00 | C |
| ATOM | 271 | O | GLN | A 64 | 14.836 | 53.071 | 6.758 | 1.00 | 0.00 | O |
| ATOM | 272 | CB | GLN | A 64 | 15.612 | 55.766 | 7.320 | 1.00 | 0.00 | C |
| ATOM | 273 | CG | GLN | A 64 | 15.512 | 57.283 | 7.347 | 1.00 | 0.00 | C |
| ATOM | 274 | CD | GLN | A 64 | 16.853 | 57.968 | 7.160 | 1.00 | 0.00 | C |
| ATOM | 275 | OE1 | GLN | A 64 | 17.819 | 57.669 | 7.868 | 1.00 | 0.00 | O |
| ATOM | 276 | NE2 | GLN | A 64 | 16.917 | 58.896 | 6.210 | 1.00 | 0.00 | N |
| ATOM | 277 | N | GLY | A 65 | 14.071 | 52.878 | 8.873 | 1.00 | 0.00 | N |
| ATOM | 278 | CA | GLY | A 65 | 14.060 | 51.428 | 8.843 | 1.00 | 0.00 | C |
| ATOM | 279 | C | GLY | A 65 | 12.713 | 50.880 | 9.283 | 1.00 | 0.00 | C |
| ATOM | 280 | O | GLY | A 65 | 11.680 | 51.204 | 8.692 | 1.00 | 0.00 | O |
| ATOM | 281 | N | TRP | A 66 | 12.723 | 50.053 | 10.323 | 1.00 | 0.00 | N |
| ATOM | 282 | CA | TRP | A 66 | 11.497 | 49.453 | 10.849 | 1.00 | 0.00 | C |
| ATOM | 283 | C | TRP | A 66 | 11.768 | 49.023 | 12.285 | 1.00 | 0.00 | C |
| ATOM | 284 | O | TRP | A 66 | 12.907 | 49.101 | 12.745 | 1.00 | 0.00 | O |
| ATOM | 285 | CB | TRP | A 66 | 11.104 | 48.234 | 10.005 | 1.00 | 0.00 | C |
| ATOM | 286 | CG | TRP | A 66 | 12.026 | 47.046 | 10.174 | 1.00 | 0.00 | C |
| ATOM | 287 | CD1 | TRP | A 66 | 11.895 | 46.027 | 11.075 | 1.00 | 0.00 | C |
| ATOM | 288 | CD2 | TRP | A 66 | 13.225 | 46.775 | 9.437 | 1.00 | 0.00 | C |
| ATOM | 289 | NE1 | TRP | A 66 | 12.934 | 45.137 | 10.944 | 1.00 | 0.00 | N |
| ATOM | 290 | CE2 | TRP | A 66 | 13.767 | 45.571 | 9.947 | 1.00 | 0.00 | C |
| ATOM | 291 | CE3 | TRP | A 66 | 13.896 | 47.432 | 8.395 | 1.00 | 0.00 | C |
| ATOM | 292 | CZ2 | TRP | A 66 | 14.950 | 45.009 | 9.450 | 1.00 | 0.00 | C |
| ATOM | 293 | CZ3 | TRP | A 66 | 15.073 | 46.874 | 7.901 | 1.00 | 0.00 | C |
| ATOM | 294 | CH2 | TRP | A 66 | 15.588 | 45.672 | 8.431 | 1.00 | 0.00 | C |
| ATOM | 295 | N | ASN | A 67 | 10.732 | 48.581 | 12.996 | 1.00 | 0.00 | N |
| ATOM | 296 | CA | ASN | A 67 | 10.908 | 48.128 | 14.375 | 1.00 | 0.00 | C |
| ATOM | 297 | C | ASN | A 67 | 11.532 | 46.739 | 14.363 | 1.00 | 0.00 | C |
| ATOM | 298 | O | ASN | A 67 | 10.861 | 45.746 | 14.070 | 1.00 | 0.00 | O |
| ATOM | 299 | CB | ASN | A 67 | 9.568 | 48.080 | 15.120 | 1.00 | 0.00 | C |
| ATOM | 300 | CG | ASN | A 67 | 8.998 | 49.458 | 15.390 | 1.00 | 0.00 | C |
| ATOM | 301 | OD1 | ASN | A 67 | 9.736 | 50.406 | 15.663 | 1.00 | 0.00 | O |
| ATOM | 302 | ND2 | ASN | A 67 | 7.676 | 49.572 | 15.337 | 1.00 | 0.00 | N |
| ATOM | 303 | N | ILE | A 68 | 12.818 | 46.669 | 14.684 | 1.00 | 0.00 | N |
| ATOM | 304 | CA | ILE | A 68 | 13.524 | 45.397 | 14.683 | 1.00 | 0.00 | C |
| ATOM | 305 | C | ILE | A 68 | 13.074 | 44.490 | 15.822 | 1.00 | 0.00 | C |
| ATOM | 306 | O | ILE | A 68 | 12.961 | 44.922 | 16.968 | 1.00 | 0.00 | O |
| ATOM | 307 | CB | ILE | A 68 | 15.046 | 45.608 | 14.799 | 1.00 | 0.00 | C |
| ATOM | 308 | CG1 | ILE | A 68 | 15.540 | 46.496 | 13.651 | 1.00 | 0.00 | C |
| ATOM | 309 | CG2 | ILE | A 68 | 15.757 | 44.265 | 14.772 | 1.00 | 0.00 | C |
| ATOM | 310 | CD1 | ILE | A 68 | 16.994 | 46.917 | 13.781 | 1.00 | 0.00 | C |
| ATOM | 311 | N | LYS | A 69 | 12.820 | 43.229 | 15.489 | 1.00 | 0.00 | N |
| ATOM | 312 | CA | LYS | A 69 | 12.402 | 42.239 | 16.474 | 1.00 | 0.00 | C |
| ATOM | 313 | C | LYS | A 69 | 13.403 | 41.090 | 16.443 | 1.00 | 0.00 | C |
| ATOM | 314 | O | LYS | A 69 | 13.979 | 40.791 | 15.399 | 1.00 | 0.00 | O |
| ATOM | 315 | CB | LYS | A 69 | 10.993 | 41.725 | 16.151 | 1.00 | 0.00 | C |
| ATOM | 316 | CG | LYS | A 69 | 9.909 | 42.788 | 16.298 | 1.00 | 0.00 | C |
| ATOM | 317 | CD | LYS | A 69 | 8.531 | 42.278 | 15.885 | 1.00 | 0.00 | C |
| ATOM | 318 | CE | LYS | A 69 | 8.478 | 41.927 | 14.402 | 1.00 | 0.00 | C |
| ATOM | 319 | NZ | LYS | A 69 | 7.079 | 41.678 | 13.933 | 1.00 | 0.00 | N |
| ATOM | 320 | N | TYR | A 70 | 13.622 | 40.458 | 17.590 | 1.00 | 0.00 | N |
| ATOM | 321 | CA | TYR | A 70 | 14.556 | 39.344 | 17.658 | 1.00 | 0.00 | C |
| ATOM | 322 | C | TYR | A 70 | 14.071 | 38.289 | 18.641 | 1.00 | 0.00 | C |
| ATOM | 323 | O | TYR | A 70 | 13.309 | 38.584 | 19.563 | 1.00 | 0.00 | O |
| ATOM | 324 | CB | TYR | A 70 | 15.952 | 39.840 | 18.063 | 1.00 | 0.00 | C |
| ATOM | 325 | CG | TYR | A 70 | 16.034 | 40.442 | 19.449 | 1.00 | 0.00 | C |
| ATOM | 326 | CD1 | TYR | A 70 | 16.268 | 39.643 | 20.569 | 1.00 | 0.00 | C |
| ATOM | 327 | CD2 | TYR | A 70 | 15.859 | 41.810 | 19.645 | 1.00 | 0.00 | C |
| ATOM | 328 | CE1 | TYR | A 70 | 16.326 | 40.194 | 21.846 | 1.00 | 0.00 | C |
| ATOM | 329 | CE2 | TYR | A 70 | 15.912 | 42.369 | 20.914 | 1.00 | 0.00 | C |
| ATOM | 330 | CZ | TYR | A 70 | 16.145 | 41.558 | 22.010 | 1.00 | 0.00 | C |
| ATOM | 331 | OH | TYR | A 70 | 16.181 | 42.115 | 23.268 | 1.00 | 0.00 | O |
| ATOM | 332 | N | ASP | A 71 | 14.507 | 37.055 | 18.423 | 1.00 | 0.00 | N |
| ATOM | 333 | CA | ASP | A 71 | 14.141 | 35.949 | 19.293 | 1.00 | 0.00 | C |
| ATOM | 334 | C | ASP | A 71 | 15.173 | 35.882 | 20.413 | 1.00 | 0.00 | C |
| ATOM | 335 | O | ASP | A 71 | 16.334 | 35.555 | 20.179 | 1.00 | 0.00 | O |
| ATOM | 336 | CB | ASP | A 71 | 14.124 | 34.644 | 18.494 | 1.00 | 0.00 | C |
| ATOM | 337 | CG | ASP | A 71 | 13.869 | 33.427 | 19.361 | 1.00 | 0.00 | C |
| ATOM | 338 | OD1 | ASP | A 71 | 13.391 | 33.590 | 20.503 | 1.00 | 0.00 | O |
| ATOM | 339 | OD2 | ASP | A 71 | 14.142 | 32.303 | 18.890 | 1.00 | 0.00 | O |
| ATOM | 340 | N | PRO | A 72 | 14.762 | 36.203 | 21.649 | 1.00 | 0.00 | N |
| ATOM | 341 | CA | PRO | A 72 | 15.688 | 36.172 | 22.785 | 1.00 | 0.00 | C |
| ATOM | 342 | C | PRO | A 72 | 16.436 | 34.852 | 22.951 | 1.00 | 0.00 | C |
| ATOM | 343 | O | PRO | A 72 | 17.546 | 34.824 | 23.486 | 1.00 | 0.00 | O |
| ATOM | 344 | CB | PRO | A 72 | 14.786 | 36.499 | 23.978 | 1.00 | 0.00 | C |
| ATOM | 345 | CG | PRO | A 72 | 13.441 | 36.007 | 23.541 | 1.00 | 0.00 | C |
| ATOM | 346 | CD | PRO | A 72 | 13.386 | 36.462 | 22.103 | 1.00 | 0.00 | C |
| ATOM | 347 | N | LEU | A 73 | 15.840 | 33.764 | 22.475 | 1.00 | 0.00 | N |
| ATOM | 348 | CA | LEU | A 73 | 16.466 | 32.453 | 22.591 | 1.00 | 0.00 | C |
| ATOM | 349 | C | LEU | A 73 | 17.534 | 32.200 | 21.533 | 1.00 | 0.00 | C |
| ATOM | 350 | O | LEU | A 73 | 18.181 | 31.155 | 21.537 | 1.00 | 0.00 | O |
| ATOM | 351 | CB | LEU | A 73 | 15.405 | 31.353 | 22.523 | 1.00 | 0.00 | C |
| ATOM | 352 | CG | LEU | A 73 | 14.411 | 31.339 | 23.687 | 1.00 | 0.00 | C |
| ATOM | 353 | CD1 | LEU | A 73 | 13.407 | 30.215 | 23.488 | 1.00 | 0.00 | C |
| ATOM | 354 | CD2 | LEU | A 73 | 15.163 | 31.163 | 25.001 | 1.00 | 0.00 | C |
| ATOM | 355 | N | LYS | A 74 | 17.724 | 33.156 | 20.630 | 1.00 | 0.00 | N |
| ATOM | 356 | CA | LYS | A 74 | 18.726 | 33.006 | 19.581 | 1.00 | 0.00 | C |
| ATOM | 357 | C | LYS | A 74 | 20.113 | 32.816 | 20.190 | 1.00 | 0.00 | C |
| ATOM | 358 | O | LYS | A 74 | 20.911 | 32.012 | 19.710 | 1.00 | 0.00 | O |
| ATOM | 359 | CB | LYS | A 74 | 18.726 | 34.233 | 18.670 | 1.00 | 0.00 | C |
| ATOM | 360 | CG | LYS | A 74 | 19.676 | 34.126 | 17.489 | 1.00 | 0.00 | C |
| ATOM | 361 | CD | LYS | A 74 | 19.504 | 35.299 | 16.540 | 1.00 | 0.00 | C |
| ATOM | 362 | CE | LYS | A 74 | 20.369 | 35.138 | 15.303 | 1.00 | 0.00 | C |
| ATOM | 363 | NZ | LYS | A 74 | 20.181 | 36.267 | 14.350 | 1.00 | 0.00 | N |
| ATOM | 364 | N | TYR | A 75 | 20.395 | 33.562 | 21.250 | 1.00 | 0.00 | N |
| ATOM | 365 | CA | TYR | A 75 | 21.683 | 33.466 | 21.919 | 1.00 | 0.00 | C |
| ATOM | 366 | C | TYR | A 75 | 21.537 | 32.733 | 23.247 | 1.00 | 0.00 | C |
| ATOM | 367 | O | TYR | A 75 | 20.536 | 32.892 | 23.944 | 1.00 | 0.00 | O |
| ATOM | 368 | CB | TYR | A 75 | 22.270 | 34.865 | 22.138 | 1.00 | 0.00 | C |
| ATOM | 369 | CG | TYR | A 75 | 22.564 | 35.588 | 20.841 | 1.00 | 0.00 | C |
| ATOM | 370 | CD1 | TYR | A 75 | 21.688 | 36.549 | 20.333 | 1.00 | 0.00 | C |
| ATOM | 371 | CD2 | TYR | A 75 | 23.689 | 35.261 | 20.087 | 1.00 | 0.00 | C |
| ATOM | 372 | CE1 | TYR | A 75 | 21.926 | 37.164 | 19.100 | 1.00 | 0.00 | C |
| ATOM | 373 | CE2 | TYR | A 75 | 23.934 | 35.865 | 18.856 | 1.00 | 0.00 | C |
| ATOM | 374 | CZ | TYR | A 75 | 23.049 | 36.812 | 18.368 | 1.00 | 0.00 | C |
| ATOM | 375 | OH | TYR | A 75 | 23.288 | 37.383 | 17.137 | 1.00 | 0.00 | O |
| ATOM | 376 | N | ASN | A 76 | 22.532 | 31.916 | 23.579 | 1.00 | 0.00 | N |
| ATOM | 377 | CA | ASN | A 76 | 22.523 | 31.153 | 24.822 | 1.00 | 0.00 | C |
| ATOM | 378 | C | ASN | A 76 | 23.951 | 30.797 | 25.225 | 1.00 | 0.00 | C |
| ATOM | 379 | O | ASN | A 76 | 24.907 | 31.191 | 24.560 | 1.00 | 0.00 | O |
| ATOM | 380 | CB | ASN | A 76 | 21.699 | 29.873 | 24.662 | 1.00 | 0.00 | C |
| ATOM | 381 | CG | ASN | A 76 | 22.393 | 28.837 | 23.806 | 1.00 | 0.00 | C |
| ATOM | 382 | OD1 | ASN | A 76 | 22.669 | 29.070 | 22.633 | 1.00 | 0.00 | O |
| ATOM | 383 | ND2 | ASN | A 76 | 22.681 | 27.680 | 24.393 | 1.00 | 0.00 | N |
| ATOM | 384 | N | ALA | A 77 | 24.086 | 30.042 | 26.312 | 1.00 | 0.00 | N |
| ATOM | 385 | CA | ALA | A 77 | 25.396 | 29.645 | 26.818 | 1.00 | 0.00 | C |
| ATOM | 386 | C | ALA | A 77 | 26.311 | 29.029 | 25.761 | 1.00 | 0.00 | C |
| ATOM | 387 | O | ALA | A 77 | 27.532 | 29.175 | 25.830 | 1.00 | 0.00 | O |
| ATOM | 388 | CB | ALA | A 77 | 25.226 | 28.673 | 27.981 | 1.00 | 0.00 | C |
| ATOM | 389 | N | HIS | A 78 | 25.725 | 28.348 | 24.784 | 1.00 | 0.00 | N |
| ATOM | 390 | CA | HIS | A 78 | 26.512 | 27.701 | 23.738 | 1.00 | 0.00 | C |
| ATOM | 391 | C | HIS | A 78 | 26.693 | 28.573 | 22.501 | 1.00 | 0.00 | C |
| ATOM | 392 | O | HIS | A 78 | 27.455 | 28.229 | 21.597 | 1.00 | 0.00 | O |
| ATOM | 393 | CB | HIS | A 78 | 25.854 | 26.376 | 23.343 | 1.00 | 0.00 | C |
| ATOM | 394 | CG | HIS | A 78 | 25.590 | 25.467 | 24.502 | 1.00 | 0.00 | C |
| ATOM | 395 | ND1 | HIS | A 78 | 26.591 | 25.016 | 25.336 | 1.00 | 0.00 | N |
| ATOM | 396 | CD2 | HIS | A 78 | 24.438 | 24.936 | 24.974 | 1.00 | 0.00 | C |
| ATOM | 397 | CE1 | HIS | A 78 | 26.066 | 24.246 | 26.273 | 1.00 | 0.00 | C |
| ATOM | 398 | NE2 | HIS | A 78 | 24.761 | 24.181 | 26.076 | 1.00 | 0.00 | N |
| ATOM | 399 | N | HIS | A 79 | 25.996 | 29.703 | 22.468 | 1.00 | 0.00 | N |
| ATOM | 400 | CA | HIS | A 79 | 26.075 | 30.618 | 21.336 | 1.00 | 0.00 | C |
| ATOM | 401 | C | HIS | A 79 | 25.850 | 32.051 | 21.821 | 1.00 | 0.00 | C |
| ATOM | 402 | O | HIS | A 79 | 24.728 | 32.551 | 21.816 | 1.00 | 0.00 | O |
| ATOM | 403 | CB | HIS | A 79 | 25.024 | 30.222 | 20.293 | 1.00 | 0.00 | C |
| ATOM | 404 | CG | HIS | A 79 | 25.102 | 31.005 | 19.020 | 1.00 | 0.00 | C |
| ATOM | 405 | ND1 | HIS | A 79 | 24.295 | 32.092 | 18.763 | 1.00 | 0.00 | N |
| ATOM | 406 | CD2 | HIS | A 79 | 25.902 | 30.865 | 17.937 | 1.00 | 0.00 | C |
| ATOM | 407 | CE1 | HIS | A 79 | 24.595 | 32.588 | 17.575 | 1.00 | 0.00 | C |
| ATOM | 408 | NE2 | HIS | A 79 | 25.567 | 31.862 | 17.054 | 1.00 | 0.00 | N |
| ATOM | 409 | N | LYS | A 80 | 26.932 | 32.699 | 22.244 | 1.00 | 0.00 | N |
| ATOM | 410 | CA | LYS | A 80 | 26.869 | 34.063 | 22.758 | 1.00 | 0.00 | C |
| ATOM | 411 | C | LYS | A 80 | 27.123 | 35.126 | 21.695 | 1.00 | 0.00 | C |
| ATOM | 412 | O | LYS | A 80 | 27.749 | 34.859 | 20.667 | 1.00 | 0.00 | O |
| ATOM | 413 | CB | LYS | A 80 | 27.900 | 34.256 | 23.872 | 1.00 | 0.00 | C |
| ATOM | 414 | CG | LYS | A 80 | 27.779 | 33.296 | 25.048 | 1.00 | 0.00 | C |
| ATOM | 415 | CD | LYS | A 80 | 28.847 | 33.614 | 26.085 | 1.00 | 0.00 | C |
| ATOM | 416 | CE | LYS | A 80 | 28.795 | 32.656 | 27.266 | 1.00 | 0.00 | C |
| ATOM | 417 | NZ | LYS | A 80 | 29.862 | 32.967 | 28.268 | 1.00 | 0.00 | N |
| ATOM | 418 | N | LEU | A 81 | 26.633 | 36.334 | 21.963 | 1.00 | 0.00 | N |
| ATOM | 419 | CA | LEU | A 81 | 26.823 | 37.470 | 21.069 | 1.00 | 0.00 | C |
| ATOM | 420 | C | LEU | A 81 | 28.096 | 38.173 | 21.528 | 1.00 | 0.00 | C |
| ATOM | 421 | O | LEU | A 81 | 28.169 | 38.662 | 22.656 | 1.00 | 0.00 | O |
| ATOM | 422 | CB | LEU | A 81 | 25.637 | 38.439 | 21.167 | 1.00 | 0.00 | C |
| ATOM | 423 | CG | LEU | A 81 | 25.716 | 39.700 | 20.296 | 1.00 | 0.00 | C |
| ATOM | 424 | CD1 | LEU | A 81 | 25.677 | 39.308 | 18.820 | 1.00 | 0.00 | C |
| ATOM | 425 | CD2 | LEU | A 81 | 24.561 | 40.635 | 20.625 | 1.00 | 0.00 | C |
| ATOM | 426 | N | LYS | A 82 | 29.104 | 38.193 | 20.663 | 1.00 | 0.00 | N |
| ATOM | 427 | CA | LYS | A 82 | 30.376 | 38.840 | 20.973 | 1.00 | 0.00 | C |
| ATOM | 428 | C | LYS | A 82 | 30.281 | 40.287 | 20.512 | 1.00 | 0.00 | C |
| ATOM | 429 | O | LYS | A 82 | 30.109 | 40.553 | 19.323 | 1.00 | 0.00 | O |
| ATOM | 430 | CB | LYS | A 82 | 31.519 | 38.128 | 20.243 | 1.00 | 0.00 | C |
| ATOM | 431 | CG | LYS | A 82 | 31.750 | 36.697 | 20.717 | 1.00 | 0.00 | C |
| ATOM | 432 | CD | LYS | A 82 | 32.635 | 35.903 | 19.758 | 1.00 | 0.00 | C |
| ATOM | 433 | CE | LYS | A 82 | 34.048 | 36.457 | 19.683 | 1.00 | 0.00 | C |
| ATOM | 434 | NZ | LYS | A 82 | 34.871 | 35.692 | 18.703 | 1.00 | 0.00 | N |
| ATOM | 435 | N | VAL | A 83 | 30.395 | 41.220 | 21.453 | 1.00 | 0.00 | N |
| ATOM | 436 | CA | VAL | A 83 | 30.291 | 42.634 | 21.125 | 1.00 | 0.00 | C |
| ATOM | 437 | C | VAL | A 83 | 31.620 | 43.372 | 21.232 | 1.00 | 0.00 | C |
| ATOM | 438 | O | VAL | A 83 | 32.304 | 43.299 | 22.253 | 1.00 | 0.00 | O |
| ATOM | 439 | CB | VAL | A 83 | 29.261 | 43.342 | 22.046 | 1.00 | 0.00 | C |
| ATOM | 440 | CG1 | VAL | A 83 | 29.131 | 44.810 | 21.662 | 1.00 | 0.00 | C |
| ATOM | 441 | CG2 | VAL | A 83 | 27.903 | 42.651 | 21.941 | 1.00 | 0.00 | C |
| ATOM | 442 | N | PHE | A 84 | 31.978 | 44.084 | 20.168 | 1.00 | 0.00 | N |
| ATOM | 443 | CA | PHE | A 84 | 33.208 | 44.868 | 20.147 | 1.00 | 0.00 | C |
| ATOM | 444 | C | PHE | A 84 | 32.880 | 46.356 | 20.069 | 1.00 | 0.00 | C |
| ATOM | 445 | O | PHE | A 84 | 32.350 | 46.823 | 19.062 | 1.00 | 0.00 | O |
| ATOM | 446 | CB | PHE | A 84 | 34.082 | 44.492 | 18.946 | 1.00 | 0.00 | C |
| ATOM | 447 | CG | PHE | A 84 | 34.769 | 43.166 | 19.083 | 1.00 | 0.00 | C |
| ATOM | 448 | CD1 | PHE | A 84 | 34.331 | 42.061 | 18.361 | 1.00 | 0.00 | C |
| ATOM | 449 | CD2 | PHE | A 84 | 35.862 | 43.023 | 19.934 | 1.00 | 0.00 | C |
| ATOM | 450 | CE1 | PHE | A 84 | 34.976 | 40.826 | 18.482 | 1.00 | 0.00 | C |
| ATOM | 451 | CE2 | PHE | A 84 | 36.515 | 41.798 | 20.063 | 1.00 | 0.00 | C |
| ATOM | 452 | CZ | PHE | A 84 | 36.072 | 40.697 | 19.337 | 1.00 | 0.00 | C |
| ATOM | 453 | N | VAL | A 85 | 33.185 | 47.084 | 21.141 | 1.00 | 0.00 | N |
| ATOM | 454 | CA | VAL | A 85 | 32.958 | 48.527 | 21.204 | 1.00 | 0.00 | C |
| ATOM | 455 | C | VAL | A 85 | 34.271 | 49.163 | 20.770 | 1.00 | 0.00 | C |
| ATOM | 456 | O | VAL | A 85 | 35.285 | 49.045 | 21.458 | 1.00 | 0.00 | O |
| ATOM | 457 | CB | VAL | A 85 | 32.601 | 48.977 | 22.639 | 1.00 | 0.00 | C |
| ATOM | 458 | CG1 | VAL | A 85 | 32.411 | 50.495 | 22.688 | 1.00 | 0.00 | C |
| ATOM | 459 | CG2 | VAL | A 85 | 31.325 | 48.272 | 23.086 | 1.00 | 0.00 | C |
| ATOM | 460 | N | VAL | A 86 | 34.242 | 49.839 | 19.627 | 1.00 | 0.00 | N |
| ATOM | 461 | CA | VAL | A 86 | 35.442 | 50.443 | 19.064 | 1.00 | 0.00 | C |
| ATOM | 462 | C | VAL | A 86 | 35.510 | 51.968 | 19.176 | 1.00 | 0.00 | C |
| ATOM | 463 | O | VAL | A 86 | 34.875 | 52.686 | 18.403 | 1.00 | 0.00 | O |
| ATOM | 464 | CB | VAL | A 86 | 35.577 | 50.032 | 17.576 | 1.00 | 0.00 | C |
| ATOM | 465 | CG1 | VAL | A 86 | 36.906 | 50.518 | 17.007 | 1.00 | 0.00 | C |
| ATOM | 466 | CG2 | VAL | A 86 | 35.451 | 48.512 | 17.445 | 1.00 | 0.00 | C |
| ATOM | 467 | N | PRO | A 87 | 36.296 | 52.477 | 20.139 | 1.00 | 0.00 | N |
| ATOM | 468 | CA | PRO | A 87 | 36.451 | 53.923 | 20.348 | 1.00 | 0.00 | C |
| ATOM | 469 | C | PRO | A 87 | 37.147 | 54.566 | 19.148 | 1.00 | 0.00 | C |
| ATOM | 470 | O | PRO | A 87 | 38.123 | 54.017 | 18.626 | 1.00 | 0.00 | O |
| ATOM | 471 | CB | PRO | A 87 | 37.309 | 54.003 | 21.613 | 1.00 | 0.00 | C |
| ATOM | 472 | CG | PRO | A 87 | 37.009 | 52.698 | 22.318 | 1.00 | 0.00 | C |
| ATOM | 473 | CD | PRO | A 87 | 37.018 | 51.724 | 21.177 | 1.00 | 0.00 | C |
| ATOM | 474 | N | HIS | A 88 | 36.649 | 55.722 | 18.715 | 1.00 | 0.00 | N |
| ATOM | 475 | CA | HIS | A 88 | 37.237 | 56.421 | 17.576 | 1.00 | 0.00 | C |
| ATOM | 476 | C | HIS | A 88 | 37.019 | 57.929 | 17.662 | 1.00 | 0.00 | C |
| ATOM | 477 | O | HIS | A 88 | 36.206 | 58.414 | 18.452 | 1.00 | 0.00 | O |
| ATOM | 478 | CB | HIS | A 88 | 36.662 | 55.876 | 16.258 | 1.00 | 0.00 | C |
| ATOM | 479 | CG | HIS | A 88 | 35.211 | 56.184 | 16.053 | 1.00 | 0.00 | C |
| ATOM | 480 | ND1 | HIS | A 88 | 34.774 | 57.311 | 15.390 | 1.00 | 0.00 | N |
| ATOM | 481 | CD2 | HIS | A 88 | 34.097 | 55.513 | 16.430 | 1.00 | 0.00 | C |
| ATOM | 482 | CE1 | HIS | A 88 | 33.454 | 57.321 | 15.367 | 1.00 | 0.00 | C |
| ATOM | 483 | NE2 | HIS | A 88 | 33.018 | 56.241 | 15.990 | 1.00 | 0.00 | N |
| ATOM | 484 | N | SER | A 89 | 37.757 | 58.664 | 16.841 | 1.00 | 0.00 | N |
| ATOM | 485 | CA | SER | A 89 | 37.676 | 60.115 | 16.827 | 1.00 | 0.00 | C |
| ATOM | 486 | C | SER | A 89 | 37.857 | 60.572 | 15.387 | 1.00 | 0.00 | C |
| ATOM | 487 | O | SER | A 89 | 38.933 | 60.414 | 14.809 | 1.00 | 0.00 | O |
| ATOM | 488 | CB | SER | A 89 | 38.779 | 60.694 | 17.724 | 1.00 | 0.00 | C |
| ATOM | 489 | OG | SER | A 89 | 38.743 | 62.109 | 17.760 | 1.00 | 0.00 | O |
| ATOM | 490 | N | HIS | A 90 | 36.797 | 61.123 | 14.809 | 1.00 | 0.00 | N |
| ATOM | 491 | CA | HIS | A 90 | 36.839 | 61.587 | 13.428 | 1.00 | 0.00 | C |
| ATOM | 492 | C | HIS | A 90 | 37.494 | 62.965 | 13.354 | 1.00 | 0.00 | C |
| ATOM | 493 | O | HIS | A 90 | 36.918 | 63.966 | 13.794 | 1.00 | 0.00 | O |
| ATOM | 494 | CB | HIS | A 90 | 35.419 | 61.634 | 12.856 | 1.00 | 0.00 | C |
| ATOM | 495 | CG | HIS | A 90 | 35.370 | 61.921 | 11.390 | 1.00 | 0.00 | C |
| ATOM | 496 | ND1 | HIS | A 90 | 35.986 | 61.118 | 10.455 | 1.00 | 0.00 | N |
| ATOM | 497 | CD2 | HIS | A 90 | 34.794 | 62.931 | 10.698 | 1.00 | 0.00 | C |
| ATOM | 498 | CE1 | HIS | A 90 | 35.793 | 61.621 | 9.250 | 1.00 | 0.00 | C |
| ATOM | 499 | NE2 | HIS | A 90 | 35.072 | 62.721 | 9.370 | 1.00 | 0.00 | N |
| ATOM | 500 | N | ASN | A 91 | 38.705 | 63.003 | 12.802 | 1.00 | 0.00 | N |
| ATOM | 501 | CA | ASN | A 91 | 39.467 | 64.242 | 12.675 | 1.00 | 0.00 | C |
| ATOM | 502 | C | ASN | A 91 | 39.630 | 64.672 | 11.223 | 1.00 | 0.00 | C |
| ATOM | 503 | O | ASN | A 91 | 40.326 | 64.019 | 10.446 | 1.00 | 0.00 | O |
| ATOM | 504 | CB | ASN | A 91 | 40.857 | 64.082 | 13.300 | 1.00 | 0.00 | C |
| ATOM | 505 | CG | ASN | A 91 | 40.812 | 63.944 | 14.807 | 1.00 | 0.00 | C |
| ATOM | 506 | OD1 | ASN | A 91 | 40.207 | 63.013 | 15.344 | 1.00 | 0.00 | O |
| ATOM | 507 | ND2 | ASN | A 91 | 41.456 | 64.872 | 15.500 | 1.00 | 0.00 | N |
| ATOM | 508 | N | ASP | A 92 | 38.996 | 65.784 | 10.871 | 1.00 | 0.00 | N |
| ATOM | 509 | CA | ASP | A 92 | 39.064 | 66.310 | 9.515 | 1.00 | 0.00 | C |
| ATOM | 510 | C | ASP | A 92 | 40.293 | 67.170 | 9.280 | 1.00 | 0.00 | C |
| ATOM | 511 | O | ASP | A 92 | 40.522 | 68.138 | 10.002 | 1.00 | 0.00 | O |
| ATOM | 512 | CB | ASP | A 92 | 37.828 | 67.157 | 9.224 | 1.00 | 0.00 | C |
| ATOM | 513 | CG | ASP | A 92 | 36.556 | 66.379 | 9.378 | 1.00 | 0.00 | C |
| ATOM | 514 | OD1 | ASP | A 92 | 36.330 | 65.473 | 8.556 | 1.00 | 0.00 | O |
| ATOM | 515 | OD2 | ASP | A 92 | 35.799 | 66.665 | 10.327 | 1.00 | 0.00 | O |
| ATOM | 516 | N | PRO | A 93 | 41.114 | 66.812 | 8.281 | 1.00 | 0.00 | N |
| ATOM | 517 | CA | PRO | A 93 | 42.311 | 67.605 | 7.983 | 1.00 | 0.00 | C |
| ATOM | 518 | C | PRO | A 93 | 41.825 | 68.880 | 7.288 | 1.00 | 0.00 | C |
| ATOM | 519 | O | PRO | A 93 | 42.162 | 69.148 | 6.133 | 1.00 | 0.00 | O |
| ATOM | 520 | CB | PRO | A 93 | 43.104 | 66.699 | 7.043 | 1.00 | 0.00 | C |
| ATOM | 521 | CG | PRO | A 93 | 42.681 | 65.313 | 7.468 | 1.00 | 0.00 | C |
| ATOM | 522 | CD | PRO | A 93 | 41.191 | 65.492 | 7.633 | 1.00 | 0.00 | C |
| ATOM | 523 | N | GLY | A 94 | 41.007 | 69.642 | 8.008 | 1.00 | 0.00 | N |
| ATOM | 524 | CA | GLY | A 94 | 40.447 | 70.870 | 7.480 | 1.00 | 0.00 | C |
| ATOM | 525 | C | GLY | A 94 | 38.966 | 70.728 | 7.157 | 1.00 | 0.00 | C |
| ATOM | 526 | O | GLY | A 94 | 38.529 | 69.702 | 6.631 | 1.00 | 0.00 | O |
| ATOM | 527 | N | TRP | A 95 | 38.193 | 71.753 | 7.507 | 1.00 | 0.00 | N |
| ATOM | 528 | CA | TRP | A 95 | 36.757 | 71.810 | 7.233 | 1.00 | 0.00 | C |
| ATOM | 529 | C | TRP | A 95 | 36.243 | 73.193 | 7.623 | 1.00 | 0.00 | C |
| ATOM | 530 | O | TRP | A 95 | 36.126 | 74.071 | 6.771 | 1.00 | 0.00 | O |
| ATOM | 531 | CB | TRP | A 95 | 35.966 | 70.740 | 7.997 | 1.00 | 0.00 | C |
| ATOM | 532 | CG | TRP | A 95 | 34.484 | 70.809 | 7.673 | 1.00 | 0.00 | C |
| ATOM | 533 | CD1 | TRP | A 95 | 33.929 | 71.139 | 6.467 | 1.00 | 0.00 | C |
| ATOM | 534 | CD2 | TRP | A 95 | 33.383 | 70.522 | 8.552 | 1.00 | 0.00 | C |
| ATOM | 535 | NE1 | TRP | A 95 | 32.557 | 71.076 | 6.539 | 1.00 | 0.00 | N |
| ATOM | 536 | CE2 | TRP | A 95 | 32.195 | 70.699 | 7.805 | 1.00 | 0.00 | C |
| ATOM | 537 | CE3 | TRP | A 95 | 33.285 | 70.129 | 9.896 | 1.00 | 0.00 | C |
| ATOM | 538 | CZ2 | TRP | A 95 | 30.922 | 70.497 | 8.356 | 1.00 | 0.00 | C |
| ATOM | 539 | CZ3 | TRP | A 95 | 32.015 | 69.927 | 10.445 | 1.00 | 0.00 | C |
| ATOM | 540 | CH2 | TRP | A 95 | 30.854 | 70.112 | 9.674 | 1.00 | 0.00 | C |
| ATOM | 541 | N | ILE | A 96 | 35.929 | 73.385 | 8.903 | 1.00 | 0.00 | N |
| ATOM | 542 | CA | ILE | A 96 | 35.467 | 74.693 | 9.363 | 1.00 | 0.00 | C |
| ATOM | 543 | C | ILE | A 96 | 36.650 | 75.452 | 9.955 | 1.00 | 0.00 | C |
| ATOM | 544 | O | ILE | A 96 | 36.557 | 76.638 | 10.273 | 1.00 | 0.00 | O |
| ATOM | 545 | CB | ILE | A 96 | 34.325 | 74.584 | 10.396 | 1.00 | 0.00 | C |
| ATOM | 546 | CG1 | ILE | A 96 | 34.748 | 73.707 | 11.571 | 1.00 | 0.00 | C |
| ATOM | 547 | CG2 | ILE | A 96 | 33.073 | 74.039 | 9.716 | 1.00 | 0.00 | C |
| ATOM | 548 | CD1 | ILE | A 96 | 33.731 | 73.670 | 12.689 | 1.00 | 0.00 | C |
| ATOM | 549 | N | GLN | A 97 | 37.763 | 74.739 | 10.096 | 1.00 | 0.00 | N |
| ATOM | 550 | CA | GLN | A 97 | 39.023 | 75.290 | 10.575 | 1.00 | 0.00 | C |
| ATOM | 551 | C | GLN | A 97 | 40.065 | 74.682 | 9.640 | 1.00 | 0.00 | C |
| ATOM | 552 | O | GLN | A 97 | 39.793 | 73.680 | 8.981 | 1.00 | 0.00 | O |
| ATOM | 553 | CB | GLN | A 97 | 39.322 | 74.857 | 12.016 | 1.00 | 0.00 | C |
| ATOM | 554 | CG | GLN | A 97 | 38.430 | 75.493 | 13.081 | 1.00 | 0.00 | C |
| ATOM | 555 | CD | GLN | A 97 | 38.920 | 75.205 | 14.494 | 1.00 | 0.00 | C |
| ATOM | 556 | OE1 | GLN | A 97 | 40.053 | 75.539 | 14.853 | 1.00 | 0.00 | O |
| ATOM | 557 | NE2 | GLN | A 97 | 38.069 | 74.580 | 15.303 | 1.00 | 0.00 | N |
| ATOM | 558 | N | THR | A 98 | 41.245 | 75.280 | 9.566 | 1.00 | 0.00 | N |
| ATOM | 559 | CA | THR | A 98 | 42.290 | 74.739 | 8.707 | 1.00 | 0.00 | C |
| ATOM | 560 | C | THR | A 98 | 42.938 | 73.549 | 9.407 | 1.00 | 0.00 | C |
| ATOM | 561 | O | THR | A 98 | 42.706 | 73.310 | 10.597 | 1.00 | 0.00 | O |
| ATOM | 562 | CB | THR | A 98 | 43.398 | 75.760 | 8.450 | 1.00 | 0.00 | C |
| ATOM | 563 | OG1 | THR | A 98 | 44.036 | 76.068 | 9.692 | 1.00 | 0.00 | O |
| ATOM | 564 | CG2 | THR | A 98 | 42.836 | 77.039 | 7.834 | 1.00 | 0.00 | C |
| ATOM | 565 | N | PHE | A 99 | 43.753 | 72.809 | 8.666 | 1.00 | 0.00 | N |
| ATOM | 566 | CA | PHE | A 99 | 44.461 | 71.664 | 9.223 | 1.00 | 0.00 | C |
| ATOM | 567 | C | PHE | A 99 | 45.209 | 72.088 | 10.488 | 1.00 | 0.00 | C |
| ATOM | 568 | O | PHE | A 99 | 45.049 | 71.481 | 11.543 | 1.00 | 0.00 | O |
| ATOM | 569 | CB | PHE | A 99 | 45.471 | 71.119 | 8.208 | 1.00 | 0.00 | C |
| ATOM | 570 | CG | PHE | A 99 | 46.348 | 70.015 | 8.749 | 1.00 | 0.00 | C |
| ATOM | 571 | CD1 | PHE | A 99 | 45.937 | 68.685 | 8.690 | 1.00 | 0.00 | C |
| ATOM | 572 | CD2 | PHE | A 99 | 47.589 | 70.307 | 9.315 | 1.00 | 0.00 | C |
| ATOM | 573 | CE1 | PHE | A 99 | 46.748 | 67.659 | 9.185 | 1.00 | 0.00 | C |
| ATOM | 574 | CE2 | PHE | A 99 | 48.408 | 69.290 | 9.813 | 1.00 | 0.00 | C |
| ATOM | 575 | CZ | PHE | A 99 | 47.985 | 67.962 | 9.746 | 1.00 | 0.00 | C |
| ATOM | 576 | N | GLU | A 100 | 46.028 | 73.131 | 10.372 | 1.00 | 0.00 | N |
| ATOM | 577 | CA | GLU | A 100 | 46.820 | 73.615 | 11.504 | 1.00 | 0.00 | C |
| ATOM | 578 | C | GLU | A 100 | 45.975 | 74.134 | 12.667 | 1.00 | 0.00 | C |
| ATOM | 579 | O | GLU | A 100 | 46.332 | 73.937 | 13.829 | 1.00 | 0.00 | O |
| ATOM | 580 | CB | GLU | A 100 | 47.789 | 74.707 | 11.037 | 1.00 | 0.00 | C |
| ATOM | 581 | CG | GLU | A 100 | 48.843 | 75.111 | 12.066 | 1.00 | 0.00 | C |
| ATOM | 582 | CD | GLU | A 100 | 49.755 | 73.964 | 12.474 | 1.00 | 0.00 | C |
| ATOM | 583 | OE1 | GLU | A 100 | 49.930 | 73.016 | 11.674 | 1.00 | 0.00 | O |
| ATOM | 584 | OE2 | GLU | A 100 | 50.313 | 74.020 | 13.593 | 1.00 | 0.00 | O |
| ATOM | 585 | N | GLU | A 101 | 44.862 | 74.798 | 12.364 | 1.00 | 0.00 | N |
| ATOM | 586 | CA | GLU | A 101 | 43.990 | 75.314 | 13.419 | 1.00 | 0.00 | C |
| ATOM | 587 | C | GLU | A 101 | 43.417 | 74.144 | 14.223 | 1.00 | 0.00 | C |
| ATOM | 588 | O | GLU | A 101 | 43.409 | 74.170 | 15.455 | 1.00 | 0.00 | O |
| ATOM | 589 | CB | GLU | A 101 | 42.850 | 76.149 | 12.821 | 1.00 | 0.00 | C |
| ATOM | 590 | CG | GLU | A 101 | 43.310 | 77.447 | 12.156 | 1.00 | 0.00 | C |
| ATOM | 591 | CD | GLU | A 101 | 42.156 | 78.271 | 11.599 | 1.00 | 0.00 | C |
| ATOM | 592 | OE1 | GLU | A 101 | 41.224 | 77.680 | 11.016 | 1.00 | 0.00 | O |
| ATOM | 593 | OE2 | GLU | A 101 | 42.191 | 79.513 | 11.735 | 1.00 | 0.00 | O |
| ATOM | 594 | N | TYR | A 102 | 42.932 | 73.120 | 13.525 | 1.00 | 0.00 | N |
| ATOM | 595 | CA | TYR | A 102 | 42.386 | 71.947 | 14.205 | 1.00 | 0.00 | C |
| ATOM | 596 | C | TYR | A 102 | 43.482 | 71.246 | 14.999 | 1.00 | 0.00 | C |
| ATOM | 597 | O | TYR | A 102 | 43.257 | 70.790 | 16.122 | 1.00 | 0.00 | O |
| ATOM | 598 | CB | TYR | A 102 | 41.821 | 70.935 | 13.209 | 1.00 | 0.00 | C |
| ATOM | 599 | CG | TYR | A 102 | 40.409 | 71.175 | 12.734 | 1.00 | 0.00 | C |
| ATOM | 600 | CD1 | TYR | A 102 | 39.355 | 71.348 | 13.637 | 1.00 | 0.00 | C |
| ATOM | 601 | CD2 | TYR | A 102 | 40.110 | 71.130 | 11.373 | 1.00 | 0.00 | C |
| ATOM | 602 | CE1 | TYR | A 102 | 38.033 | 71.459 | 13.185 | 1.00 | 0.00 | C |
| ATOM | 603 | CE2 | TYR | A 102 | 38.810 | 71.239 | 10.917 | 1.00 | 0.00 | C |
| ATOM | 604 | CZ | TYR | A 102 | 37.775 | 71.401 | 11.821 | 1.00 | 0.00 | C |
| ATOM | 605 | OH | TYR | A 102 | 36.493 | 71.491 | 11.335 | 1.00 | 0.00 | O |
| ATOM | 606 | N | TYR | A 103 | 44.664 | 71.137 | 14.403 | 1.00 | 0.00 | N |
| ATOM | 607 | CA | TYR | A 103 | 45.764 | 70.470 | 15.080 | 1.00 | 0.00 | C |
| ATOM | 608 | C | TYR | A 103 | 46.086 | 71.147 | 16.405 | 1.00 | 0.00 | C |
| ATOM | 609 | O | TYR | A 103 | 46.216 | 70.488 | 17.434 | 1.00 | 0.00 | O |
| ATOM | 610 | CB | TYR | A 103 | 47.024 | 70.466 | 14.216 | 1.00 | 0.00 | C |
| ATOM | 611 | CG | TYR | A 103 | 48.190 | 69.808 | 14.915 | 1.00 | 0.00 | C |
| ATOM | 612 | CD1 | TYR | A 103 | 48.194 | 68.433 | 15.158 | 1.00 | 0.00 | C |
| ATOM | 613 | CD2 | TYR | A 103 | 49.262 | 70.565 | 15.392 | 1.00 | 0.00 | C |
| ATOM | 614 | CE1 | TYR | A 103 | 49.233 | 67.830 | 15.861 | 1.00 | 0.00 | C |
| ATOM | 615 | CE2 | TYR | A 103 | 50.305 | 69.972 | 16.098 | 1.00 | 0.00 | C |
| ATOM | 616 | CZ | TYR | A 103 | 50.284 | 68.605 | 16.330 | 1.00 | 0.00 | C |
| ATOM | 617 | OH | TYR | A 103 | 51.305 | 68.014 | 17.037 | 1.00 | 0.00 | O |
| ATOM | 618 | N | GLN | A 104 | 46.212 | 72.468 | 16.372 | 1.00 | 0.00 | N |
| ATOM | 619 | CA | GLN | A 104 | 46.544 | 73.231 | 17.568 | 1.00 | 0.00 | C |
| ATOM | 620 | C | GLN | A 104 | 45.438 | 73.258 | 18.611 | 1.00 | 0.00 | C |
| ATOM | 621 | O | GLN | A 104 | 45.705 | 73.155 | 19.806 | 1.00 | 0.00 | O |
| ATOM | 622 | CB | GLN | A 104 | 46.906 | 74.675 | 17.190 | 1.00 | 0.00 | C |
| ATOM | 623 | CG | GLN | A 104 | 48.224 | 74.829 | 16.440 | 1.00 | 0.00 | C |
| ATOM | 624 | CD | GLN | A 104 | 49.410 | 74.290 | 17.225 | 1.00 | 0.00 | C |
| ATOM | 625 | OE1 | GLN | A 104 | 49.459 | 74.399 | 18.454 | 1.00 | 0.00 | O |
| ATOM | 626 | NE2 | GLN | A 104 | 50.378 | 73.714 | 16.518 | 1.00 | 0.00 | N |
| ATOM | 627 | N | HIS | A 105 | 44.197 | 73.389 | 18.161 | 1.00 | 0.00 | N |
| ATOM | 628 | CA | HIS | A 105 | 43.067 | 73.477 | 19.077 | 1.00 | 0.00 | C |
| ATOM | 629 | C | HIS | A 105 | 42.492 | 72.152 | 19.566 | 1.00 | 0.00 | C |
| ATOM | 630 | O | HIS | A 105 | 42.010 | 72.070 | 20.694 | 1.00 | 0.00 | O |
| ATOM | 631 | CB | HIS | A 105 | 41.935 | 74.279 | 18.426 | 1.00 | 0.00 | C |
| ATOM | 632 | CG | HIS | A 105 | 42.367 | 75.595 | 17.856 | 1.00 | 0.00 | C |
| ATOM | 633 | ND1 | HIS | A 105 | 41.509 | 76.421 | 17.162 | 1.00 | 0.00 | N |
| ATOM | 634 | CD2 | HIS | A 105 | 43.566 | 76.226 | 17.872 | 1.00 | 0.00 | C |
| ATOM | 635 | CE1 | HIS | A 105 | 42.161 | 77.503 | 16.774 | 1.00 | 0.00 | C |
| ATOM | 636 | NE2 | HIS | A 105 | 43.410 | 77.409 | 17.192 | 1.00 | 0.00 | N |
| ATOM | 637 | N | ASP | A 106 | 42.549 | 71.115 | 18.733 | 1.00 | 0.00 | N |
| ATOM | 638 | CA | ASP | A 106 | 41.951 | 69.837 | 19.104 | 1.00 | 0.00 | C |
| ATOM | 639 | C | ASP | A 106 | 42.786 | 68.568 | 18.974 | 1.00 | 0.00 | C |
| ATOM | 640 | O | ASP | A 106 | 43.052 | 67.887 | 19.961 | 1.00 | 0.00 | O |
| ATOM | 641 | CB | ASP | A 106 | 40.662 | 69.641 | 18.296 | 1.00 | 0.00 | C |
| ATOM | 642 | CG | ASP | A 106 | 39.644 | 70.735 | 18.545 | 1.00 | 0.00 | C |
| ATOM | 643 | OD1 | ASP | A 106 | 38.958 | 70.682 | 19.585 | 1.00 | 0.00 | O |
| ATOM | 644 | OD2 | ASP | A 106 | 39.539 | 71.654 | 17.705 | 1.00 | 0.00 | O |
| ATOM | 645 | N | THR | A 107 | 43.188 | 68.252 | 17.749 | 1.00 | 0.00 | N |
| ATOM | 646 | CA | THR | A 107 | 43.928 | 67.027 | 17.469 | 1.00 | 0.00 | C |
| ATOM | 647 | C | THR | A 107 | 45.179 | 66.710 | 18.286 | 1.00 | 0.00 | C |
| ATOM | 648 | O | THR | A 107 | 45.365 | 65.565 | 18.702 | 1.00 | 0.00 | O |
| ATOM | 649 | CB | THR | A 107 | 44.287 | 66.947 | 15.981 | 1.00 | 0.00 | C |
| ATOM | 650 | OG1 | THR | A 107 | 43.103 | 67.152 | 15.199 | 1.00 | 0.00 | O |
| ATOM | 651 | CG2 | THR | A 107 | 44.868 | 65.570 | 15.648 | 1.00 | 0.00 | C |
| ATOM | 652 | N | LYS | A 108 | 46.048 | 67.687 | 18.517 | 1.00 | 0.00 | N |
| ATOM | 653 | CA | LYS | A 108 | 47.249 | 67.367 | 19.280 | 1.00 | 0.00 | C |
| ATOM | 654 | C | LYS | A 108 | 46.887 | 66.969 | 20.707 | 1.00 | 0.00 | C |
| ATOM | 655 | O | LYS | A 108 | 47.585 | 66.176 | 21.331 | 1.00 | 0.00 | O |
| ATOM | 656 | CB | LYS | A 108 | 48.247 | 68.537 | 19.273 | 1.00 | 0.00 | C |
| ATOM | 657 | CG | LYS | A 108 | 47.856 | 69.754 | 20.088 | 1.00 | 0.00 | C |
| ATOM | 658 | CD | LYS | A 108 | 48.942 | 70.826 | 19.973 | 1.00 | 0.00 | C |
| ATOM | 659 | CE | LYS | A 108 | 48.713 | 71.978 | 20.937 | 1.00 | 0.00 | C |
| ATOM | 660 | NZ | LYS | A 108 | 49.823 | 72.978 | 20.873 | 1.00 | 0.00 | N |
| ATOM | 661 | N | HIS | A 109 | 45.786 | 67.510 | 21.216 | 1.00 | 0.00 | N |
| ATOM | 662 | CA | HIS | A 109 | 45.343 | 67.184 | 22.567 | 1.00 | 0.00 | C |
| ATOM | 663 | C | HIS | A 109 | 44.701 | 65.802 | 22.575 | 1.00 | 0.00 | C |
| ATOM | 664 | O | HIS | A 109 | 44.871 | 65.029 | 23.518 | 1.00 | 0.00 | O |
| ATOM | 665 | CB | HIS | A 109 | 44.352 | 68.236 | 23.055 | 1.00 | 0.00 | C |
| ATOM | 666 | CG | HIS | A 109 | 44.923 | 69.617 | 23.085 | 1.00 | 0.00 | C |
| ATOM | 667 | ND1 | HIS | A 109 | 45.939 | 69.982 | 23.944 | 1.00 | 0.00 | N |
| ATOM | 668 | CD2 | HIS | A 109 | 44.652 | 70.711 | 22.335 | 1.00 | 0.00 | C |
| ATOM | 669 | CE1 | HIS | A 109 | 46.268 | 71.243 | 23.721 | 1.00 | 0.00 | C |
| ATOM | 670 | NE2 | HIS | A 109 | 45.502 | 71.707 | 22.749 | 1.00 | 0.00 | N |
| ATOM | 671 | N | ILE | A 110 | 43.966 | 65.496 | 21.513 | 1.00 | 0.00 | N |
| ATOM | 672 | CA | ILE | A 110 | 43.319 | 64.198 | 21.382 | 1.00 | 0.00 | C |
| ATOM | 673 | C | ILE | A 110 | 44.378 | 63.099 | 21.355 | 1.00 | 0.00 | C |
| ATOM | 674 | O | ILE | A 110 | 44.257 | 62.084 | 22.040 | 1.00 | 0.00 | O |
| ATOM | 675 | CB | ILE | A 110 | 42.492 | 64.123 | 20.078 | 1.00 | 0.00 | C |
| ATOM | 676 | CG1 | ILE | A 110 | 41.302 | 65.086 | 20.167 | 1.00 | 0.00 | C |
| ATOM | 677 | CG2 | ILE | A 110 | 42.043 | 62.688 | 19.826 | 1.00 | 0.00 | C |
| ATOM | 678 | CD1 | ILE | A 110 | 40.511 | 65.224 | 18.870 | 1.00 | 0.00 | C |
| ATOM | 679 | N | LEU | A 111 | 45.422 | 63.305 | 20.562 | 1.00 | 0.00 | N |
| ATOM | 680 | CA | LEU | A 111 | 46.481 | 62.313 | 20.456 | 1.00 | 0.00 | C |
| ATOM | 681 | C | LEU | A 111 | 47.341 | 62.244 | 21.708 | 1.00 | 0.00 | C |
| ATOM | 682 | O | LEU | A 111 | 47.783 | 61.163 | 22.105 | 1.00 | 0.00 | O |
| ATOM | 683 | CB | LEU | A 111 | 47.348 | 62.597 | 19.222 | 1.00 | 0.00 | C |
| ATOM | 684 | CG | LEU | A 111 | 46.620 | 62.277 | 17.911 | 1.00 | 0.00 | C |
| ATOM | 685 | CD1 | LEU | A 111 | 47.442 | 62.742 | 16.715 | 1.00 | 0.00 | C |
| ATOM | 686 | CD2 | LEU | A 111 | 46.352 | 60.774 | 17.845 | 1.00 | 0.00 | C |
| ATOM | 687 | N | SER | A 112 | 47.573 | 63.390 | 22.337 | 1.00 | 0.00 | N |
| ATOM | 688 | CA | SER | A 112 | 48.381 | 63.412 | 23.549 | 1.00 | 0.00 | C |
| ATOM | 689 | C | SER | A 112 | 47.666 | 62.672 | 24.679 | 1.00 | 0.00 | C |
| ATOM | 690 | O | SER | A 112 | 48.272 | 61.870 | 25.395 | 1.00 | 0.00 | O |
| ATOM | 691 | CB | SER | A 112 | 48.670 | 64.853 | 23.971 | 1.00 | 0.00 | C |
| ATOM | 692 | OG | SER | A 112 | 49.509 | 64.872 | 25.113 | 1.00 | 0.00 | O |
| ATOM | 693 | N | ASN | A 113 | 46.373 | 62.933 | 24.838 | 1.00 | 0.00 | N |
| ATOM | 694 | CA | ASN | A 113 | 45.625 | 62.265 | 25.891 | 1.00 | 0.00 | C |
| ATOM | 695 | C | ASN | A 113 | 45.330 | 60.802 | 25.556 | 1.00 | 0.00 | C |
| ATOM | 696 | O | ASN | A 113 | 45.143 | 59.982 | 26.457 | 1.00 | 0.00 | O |
| ATOM | 697 | CB | ASN | A 113 | 44.352 | 63.052 | 26.209 | 1.00 | 0.00 | C |
| ATOM | 698 | CG | ASN | A 113 | 44.666 | 64.403 | 26.833 | 1.00 | 0.00 | C |
| ATOM | 699 | OD1 | ASN | A 113 | 45.664 | 64.541 | 27.538 | 1.00 | 0.00 | O |
| ATOM | 700 | ND2 | ASN | A 113 | 43.829 | 65.397 | 26.582 | 1.00 | 0.00 | N |
| ATOM | 701 | N | ALA | A 114 | 45.310 | 60.467 | 24.268 | 1.00 | 0.00 | N |
| ATOM | 702 | CA | ALA | A 114 | 45.081 | 59.080 | 23.866 | 1.00 | 0.00 | C |
| ATOM | 703 | C | ALA | A 114 | 46.296 | 58.269 | 24.305 | 1.00 | 0.00 | C |
| ATOM | 704 | O | ALA | A 114 | 46.160 | 57.174 | 24.853 | 1.00 | 0.00 | O |
| ATOM | 705 | CB | ALA | A 114 | 44.903 | 58.978 | 22.349 | 1.00 | 0.00 | C |
| ATOM | 706 | N | LEU | A 115 | 47.487 | 58.814 | 24.064 | 1.00 | 0.00 | N |
| ATOM | 707 | CA | LEU | A 115 | 48.725 | 58.143 | 24.445 | 1.00 | 0.00 | C |
| ATOM | 708 | C | LEU | A 115 | 48.744 | 57.892 | 25.953 | 1.00 | 0.00 | C |
| ATOM | 709 | O | LEU | A 115 | 49.029 | 56.782 | 26.408 | 1.00 | 0.00 | O |
| ATOM | 710 | CB | LEU | A 115 | 49.942 | 58.995 | 24.046 | 1.00 | 0.00 | C |
| ATOM | 711 | CG | LEU | A 115 | 51.322 | 58.485 | 24.481 | 1.00 | 0.00 | C |
| ATOM | 712 | CD1 | LEU | A 115 | 51.544 | 57.075 | 23.953 | 1.00 | 0.00 | C |
| ATOM | 713 | CD2 | LEU | A 115 | 52.408 | 59.419 | 23.978 | 1.00 | 0.00 | C |
| ATOM | 714 | N | ARG | A 116 | 48.421 | 58.925 | 26.721 | 1.00 | 0.00 | N |
| ATOM | 715 | CA | ARG | A 116 | 48.412 | 58.813 | 28.173 | 1.00 | 0.00 | C |
| ATOM | 716 | C | ARG | A 116 | 47.380 | 57.811 | 28.677 | 1.00 | 0.00 | C |
| ATOM | 717 | O | ARG | A 116 | 47.704 | 56.900 | 29.442 | 1.00 | 0.00 | O |
| ATOM | 718 | CB | ARG | A 116 | 48.152 | 60.185 | 28.797 | 1.00 | 0.00 | C |
| ATOM | 719 | CG | ARG | A 116 | 49.268 | 61.180 | 28.533 | 1.00 | 0.00 | C |
| ATOM | 720 | CD | ARG | A 116 | 48.932 | 62.575 | 29.033 | 1.00 | 0.00 | C |
| ATOM | 721 | NE | ARG | A 116 | 50.033 | 63.508 | 28.802 | 1.00 | 0.00 | N |
| ATOM | 722 | CZ | ARG | A 116 | 49.984 | 64.808 | 29.075 | 1.00 | 0.00 | C |
| ATOM | 723 | NH1 | ARG | A 116 | 48.882 | 65.338 | 29.590 | 1.00 | 0.00 | N |
| ATOM | 724 | NH2 | ARG | A 116 | 51.039 | 65.579 | 28.838 | 1.00 | 0.00 | N |
| ATOM | 725 | N | HIS | A 117 | 46.138 | 57.971 | 28.236 | 1.00 | 0.00 | N |
| ATOM | 726 | CA | HIS | A 117 | 45.070 | 57.085 | 28.670 | 1.00 | 0.00 | C |
| ATOM | 727 | C | HIS | A 117 | 45.204 | 55.628 | 28.265 | 1.00 | 0.00 | C |
| ATOM | 728 | O | HIS | A 117 | 44.892 | 54.745 | 29.059 | 1.00 | 0.00 | O |
| ATOM | 729 | CB | HIS | A 117 | 43.726 | 57.651 | 28.230 | 1.00 | 0.00 | C |
| ATOM | 730 | CG | HIS | A 117 | 43.233 | 58.737 | 29.130 | 1.00 | 0.00 | C |
| ATOM | 731 | ND1 | HIS | A 117 | 42.625 | 58.474 | 30.339 | 1.00 | 0.00 | N |
| ATOM | 732 | CD2 | HIS | A 117 | 43.355 | 60.083 | 29.054 | 1.00 | 0.00 | C |
| ATOM | 733 | CE1 | HIS | A 117 | 42.399 | 59.612 | 30.973 | 1.00 | 0.00 | C |
| ATOM | 734 | NE2 | HIS | A 117 | 42.834 | 60.604 | 30.216 | 1.00 | 0.00 | N |
| ATOM | 735 | N | LEU | A 118 | 45.662 | 55.366 | 27.044 | 1.00 | 0.00 | N |
| ATOM | 736 | CA | LEU | A 118 | 45.839 | 53.986 | 26.606 | 1.00 | 0.00 | C |
| ATOM | 737 | C | LEU | A 118 | 47.012 | 53.379 | 27.368 | 1.00 | 0.00 | C |
| ATOM | 738 | O | LEU | A 118 | 46.987 | 52.208 | 27.747 | 1.00 | 0.00 | O |
| ATOM | 739 | CB | LEU | A 118 | 46.099 | 53.924 | 25.095 | 1.00 | 0.00 | C |
| ATOM | 740 | CG | LEU | A 118 | 44.915 | 54.378 | 24.230 | 1.00 | 0.00 | C |
| ATOM | 741 | CD1 | LEU | A 118 | 45.301 | 54.355 | 22.759 | 1.00 | 0.00 | C |
| ATOM | 742 | CD2 | LEU | A 118 | 43.722 | 53.470 | 24.484 | 1.00 | 0.00 | C |
| ATOM | 743 | N | HIS | A 119 | 48.047 | 54.180 | 27.589 | 1.00 | 0.00 | N |
| ATOM | 744 | CA | HIS | A 119 | 49.210 | 53.707 | 28.321 | 1.00 | 0.00 | C |
| ATOM | 745 | C | HIS | A 119 | 48.791 | 53.245 | 29.717 | 1.00 | 0.00 | C |
| ATOM | 746 | O | HIS | A 119 | 49.170 | 52.160 | 30.159 | 1.00 | 0.00 | O |
| ATOM | 747 | CB | HIS | A 119 | 50.257 | 54.826 | 28.422 | 1.00 | 0.00 | C |
| ATOM | 748 | CG | HIS | A 119 | 51.418 | 54.493 | 29.307 | 1.00 | 0.00 | C |
| ATOM | 749 | ND1 | HIS | A 119 | 51.416 | 54.745 | 30.662 | 1.00 | 0.00 | N |
| ATOM | 750 | CD2 | HIS | A 119 | 52.603 | 53.897 | 29.036 | 1.00 | 0.00 | C |
| ATOM | 751 | CE1 | HIS | A 119 | 52.551 | 54.319 | 31.189 | 1.00 | 0.00 | C |
| ATOM | 752 | NE2 | HIS | A 119 | 53.289 | 53.799 | 30.224 | 1.00 | 0.00 | N |
| ATOM | 753 | N | ASP | A 120 | 47.984 | 54.060 | 30.391 | 1.00 | 0.00 | N |
| ATOM | 754 | CA | ASP | A 120 | 47.527 | 53.762 | 31.750 | 1.00 | 0.00 | C |
| ATOM | 755 | C | ASP | A 120 | 46.370 | 52.775 | 31.895 | 1.00 | 0.00 | C |
| ATOM | 756 | O | ASP | A 120 | 46.126 | 52.274 | 32.993 | 1.00 | 0.00 | O |
| ATOM | 757 | CB | ASP | A 120 | 47.147 | 55.057 | 32.469 | 1.00 | 0.00 | C |
| ATOM | 758 | CG | ASP | A 120 | 48.324 | 55.989 | 32.653 | 1.00 | 0.00 | C |
| ATOM | 759 | OD1 | ASP | A 120 | 49.475 | 55.505 | 32.673 | 1.00 | 0.00 | O |
| ATOM | 760 | OD2 | ASP | A 120 | 48.094 | 57.207 | 32.793 | 1.00 | 0.00 | O |
| ATOM | 761 | N | ASN | A 121 | 45.656 | 52.501 | 30.807 | 1.00 | 0.00 | N |
| ATOM | 762 | CA | ASN | A 121 | 44.523 | 51.574 | 30.849 | 1.00 | 0.00 | C |
| ATOM | 763 | C | ASN | A 121 | 44.681 | 50.515 | 29.758 | 1.00 | 0.00 | C |
| ATOM | 764 | O | ASN | A 121 | 44.105 | 50.630 | 28.674 | 1.00 | 0.00 | O |
| ATOM | 765 | CB | ASN | A 121 | 43.210 | 52.343 | 30.661 | 1.00 | 0.00 | C |
| ATOM | 766 | CG | ASN | A 121 | 43.007 | 53.423 | 31.719 | 1.00 | 0.00 | C |
| ATOM | 767 | OD1 | ASN | A 121 | 43.448 | 54.567 | 31.558 | 1.00 | 0.00 | O |
| ATOM | 768 | ND2 | ASN | A 121 | 42.348 | 53.059 | 32.813 | 1.00 | 0.00 | N |
| ATOM | 769 | N | PRO | A 122 | 45.454 | 49.452 | 30.046 | 1.00 | 0.00 | N |
| ATOM | 770 | CA | PRO | A 122 | 45.732 | 48.346 | 29.122 | 1.00 | 0.00 | C |
| ATOM | 771 | C | PRO | A 122 | 44.580 | 47.753 | 28.311 | 1.00 | 0.00 | C |
| ATOM | 772 | O | PRO | A 122 | 44.798 | 47.303 | 27.184 | 1.00 | 0.00 | O |
| ATOM | 773 | CB | PRO | A 122 | 46.426 | 47.311 | 30.017 | 1.00 | 0.00 | C |
| ATOM | 774 | CG | PRO | A 122 | 45.937 | 47.647 | 31.399 | 1.00 | 0.00 | C |
| ATOM | 775 | CD | PRO | A 122 | 45.974 | 49.145 | 31.389 | 1.00 | 0.00 | C |
| ATOM | 776 | N | GLU | A 123 | 43.366 | 47.753 | 28.858 | 1.00 | 0.00 | N |
| ATOM | 777 | CA | GLU | A 123 | 42.224 | 47.192 | 28.135 | 1.00 | 0.00 | C |
| ATOM | 778 | C | GLU | A 123 | 41.503 | 48.170 | 27.215 | 1.00 | 0.00 | C |
| ATOM | 779 | O | GLU | A 123 | 40.678 | 47.759 | 26.398 | 1.00 | 0.00 | O |
| ATOM | 780 | CB | GLU | A 123 | 41.204 | 46.591 | 29.105 | 1.00 | 0.00 | C |
| ATOM | 781 | CG | GLU | A 123 | 41.476 | 45.145 | 29.477 | 1.00 | 0.00 | C |
| ATOM | 782 | CD | GLU | A 123 | 42.676 | 44.989 | 30.375 | 1.00 | 0.00 | C |
| ATOM | 783 | OE1 | GLU | A 123 | 42.678 | 45.605 | 31.463 | 1.00 | 0.00 | O |
| ATOM | 784 | OE2 | GLU | A 123 | 43.613 | 44.249 | 29.999 | 1.00 | 0.00 | O |
| ATOM | 785 | N | MET | A 124 | 41.798 | 49.458 | 27.352 | 1.00 | 0.00 | N |
| ATOM | 786 | CA | MET | A 124 | 41.165 | 50.464 | 26.506 | 1.00 | 0.00 | C |
| ATOM | 787 | C | MET | A 124 | 41.742 | 50.354 | 25.092 | 1.00 | 0.00 | C |
| ATOM | 788 | O | MET | A 124 | 42.918 | 50.024 | 24.919 | 1.00 | 0.00 | O |
| ATOM | 789 | CB | MET | A 124 | 41.418 | 51.864 | 27.068 | 1.00 | 0.00 | C |
| ATOM | 790 | CG | MET | A 124 | 40.644 | 52.963 | 26.354 | 1.00 | 0.00 | C |
| ATOM | 791 | SD | MET | A 124 | 38.864 | 52.639 | 26.329 | 1.00 | 0.00 | S |
| ATOM | 792 | CE | MET | A 124 | 38.255 | 54.154 | 25.577 | 1.00 | 0.00 | C |
| ATOM | 793 | N | LYS | A 125 | 40.908 | 50.624 | 24.090 | 1.00 | 0.00 | N |
| ATOM | 794 | CA | LYS | A 125 | 41.322 | 50.546 | 22.691 | 1.00 | 0.00 | C |
| ATOM | 795 | C | LYS | A 125 | 40.980 | 51.842 | 21.954 | 1.00 | 0.00 | C |
| ATOM | 796 | O | LYS | A 125 | 40.178 | 52.646 | 22.434 | 1.00 | 0.00 | O |
| ATOM | 797 | CB | LYS | A 125 | 40.623 | 49.360 | 22.018 | 1.00 | 0.00 | C |
| ATOM | 798 | CG | LYS | A 125 | 40.950 | 48.006 | 22.643 | 1.00 | 0.00 | C |
| ATOM | 799 | CD | LYS | A 125 | 42.347 | 47.539 | 22.267 | 1.00 | 0.00 | C |
| ATOM | 800 | CE | LYS | A 125 | 42.747 | 46.273 | 23.022 | 1.00 | 0.00 | C |
| ATOM | 801 | NZ | LYS | A 125 | 41.818 | 45.136 | 22.781 | 1.00 | 0.00 | N |
| ATOM | 802 | N | PHE | A 126 | 41.569 | 52.039 | 20.778 | 1.00 | 0.00 | N |
| ATOM | 803 | CA | PHE | A 126 | 41.324 | 53.264 | 20.016 | 1.00 | 0.00 | C |
| ATOM | 804 | C | PHE | A 126 | 41.824 | 53.081 | 18.582 | 1.00 | 0.00 | C |
| ATOM | 805 | O | PHE | A 126 | 42.900 | 52.529 | 18.366 | 1.00 | 0.00 | O |
| ATOM | 806 | CB | PHE | A 126 | 42.077 | 54.418 | 20.698 | 1.00 | 0.00 | C |
| ATOM | 807 | CG | PHE | A 126 | 41.706 | 55.797 | 20.203 | 1.00 | 0.00 | C |
| ATOM | 808 | CD1 | PHE | A 126 | 40.384 | 56.230 | 20.214 | 1.00 | 0.00 | C |
| ATOM | 809 | CD2 | PHE | A 126 | 42.699 | 56.689 | 19.802 | 1.00 | 0.00 | C |
| ATOM | 810 | CE1 | PHE | A 126 | 40.054 | 57.536 | 19.838 | 1.00 | 0.00 | C |
| ATOM | 811 | CE2 | PHE | A 126 | 42.384 | 57.997 | 19.423 | 1.00 | 0.00 | C |
| ATOM | 812 | CZ | PHE | A 126 | 41.057 | 58.422 | 19.442 | 1.00 | 0.00 | C |
| ATOM | 813 | N | ILE | A 127 | 41.042 | 53.526 | 17.602 | 1.00 | 0.00 | N |
| ATOM | 814 | CA | ILE | A 127 | 41.471 | 53.418 | 16.210 | 1.00 | 0.00 | C |
| ATOM | 815 | C | ILE | A 127 | 41.733 | 54.811 | 15.640 | 1.00 | 0.00 | C |
| ATOM | 816 | O | ILE | A 127 | 41.067 | 55.782 | 16.021 | 1.00 | 0.00 | O |
| ATOM | 817 | CB | ILE | A 127 | 40.427 | 52.687 | 15.338 | 1.00 | 0.00 | C |
| ATOM | 818 | CG1 | ILE | A 127 | 39.089 | 53.433 | 15.363 | 1.00 | 0.00 | C |
| ATOM | 819 | CG2 | ILE | A 127 | 40.258 | 51.260 | 15.840 | 1.00 | 0.00 | C |
| ATOM | 820 | CD1 | ILE | A 127 | 38.066 | 52.870 | 14.392 | 1.00 | 0.00 | C |
| ATOM | 821 | N | TRP | A 128 | 42.713 | 54.910 | 14.745 | 1.00 | 0.00 | N |
| ATOM | 822 | CA | TRP | A 128 | 43.061 | 56.193 | 14.135 | 1.00 | 0.00 | C |
| ATOM | 823 | C | TRP | A 128 | 43.178 | 56.076 | 12.617 | 1.00 | 0.00 | C |
| ATOM | 824 | O | TRP | A 128 | 43.820 | 55.161 | 12.105 | 1.00 | 0.00 | O |
| ATOM | 825 | CB | TRP | A 128 | 44.372 | 56.719 | 14.716 | 1.00 | 0.00 | C |
| ATOM | 826 | CG | TRP | A 128 | 44.611 | 58.146 | 14.356 | 1.00 | 0.00 | C |
| ATOM | 827 | CD1 | TRP | A 128 | 45.351 | 58.621 | 13.309 | 1.00 | 0.00 | C |
| ATOM | 828 | CD2 | TRP | A 128 | 44.053 | 59.290 | 15.006 | 1.00 | 0.00 | C |
| ATOM | 829 | NE1 | TRP | A 128 | 45.287 | 59.995 | 13.269 | 1.00 | 0.00 | N |
| ATOM | 830 | CE2 | TRP | A 128 | 44.495 | 60.431 | 14.300 | 1.00 | 0.00 | C |
| ATOM | 831 | CE3 | TRP | A 128 | 43.216 | 59.464 | 16.120 | 1.00 | 0.00 | C |
| ATOM | 832 | CZ2 | TRP | A 128 | 44.130 | 61.731 | 14.670 | 1.00 | 0.00 | C |
| ATOM | 833 | CZ3 | TRP | A 128 | 42.852 | 60.758 | 16.486 | 1.00 | 0.00 | C |
| ATOM | 834 | CH2 | TRP | A 128 | 43.311 | 61.873 | 15.761 | 1.00 | 0.00 | C |
| ATOM | 835 | N | ALA | A 129 | 42.582 | 57.027 | 11.900 | 1.00 | 0.00 | N |
| ATOM | 836 | CA | ALA | A 129 | 42.587 | 56.974 | 10.439 | 1.00 | 0.00 | C |
| ATOM | 837 | C | ALA | A 129 | 43.438 | 57.984 | 9.672 | 1.00 | 0.00 | C |
| ATOM | 838 | O | ALA | A 129 | 44.069 | 57.625 | 8.681 | 1.00 | 0.00 | O |
| ATOM | 839 | CB | ALA | A 129 | 41.150 | 57.054 | 9.933 | 1.00 | 0.00 | C |
| ATOM | 840 | N | GLU | A 130 | 43.454 | 59.234 | 10.123 | 1.00 | 0.00 | N |
| ATOM | 841 | CA | GLU | A 130 | 44.180 | 60.299 | 9.427 | 1.00 | 0.00 | C |
| ATOM | 842 | C | GLU | A 130 | 45.665 | 60.421 | 9.757 | 1.00 | 0.00 | C |
| ATOM | 843 | O | GLU | A 130 | 46.045 | 61.041 | 10.752 | 1.00 | 0.00 | O |
| ATOM | 844 | CB | GLU | A 130 | 43.492 | 61.641 | 9.688 | 1.00 | 0.00 | C |
| ATOM | 845 | CG | GLU | A 130 | 42.001 | 61.660 | 9.373 | 1.00 | 0.00 | C |
| ATOM | 846 | CD | GLU | A 130 | 41.151 | 61.055 | 10.480 | 1.00 | 0.00 | C |
| ATOM | 847 | OE1 | GLU | A 130 | 41.701 | 60.758 | 11.563 | 1.00 | 0.00 | O |
| ATOM | 848 | OE2 | GLU | A 130 | 39.930 | 60.888 | 10.269 | 1.00 | 0.00 | O |
| ATOM | 849 | N | ILE | A 131 | 46.509 | 59.870 | 8.890 | 1.00 | 0.00 | N |
| ATOM | 850 | CA | ILE | A 131 | 47.945 | 59.908 | 9.128 | 1.00 | 0.00 | C |
| ATOM | 851 | C | ILE | A 131 | 48.564 | 61.304 | 9.007 | 1.00 | 0.00 | C |
| ATOM | 852 | O | ILE | A 131 | 49.593 | 61.574 | 9.632 | 1.00 | 0.00 | O |
| ATOM | 853 | CB | ILE | A 131 | 48.676 | 58.906 | 8.207 | 1.00 | 0.00 | C |
| ATOM | 854 | CG1 | ILE | A 131 | 48.061 | 57.512 | 8.393 | 1.00 | 0.00 | C |
| ATOM | 855 | CG2 | ILE | A 131 | 50.161 | 58.851 | 8.553 | 1.00 | 0.00 | C |
| ATOM | 856 | CD1 | ILE | A 131 | 47.933 | 57.084 | 9.857 | 1.00 | 0.00 | C |
| ATOM | 857 | N | SER | A 132 | 47.946 | 62.191 | 8.227 | 1.00 | 0.00 | N |
| ATOM | 858 | CA | SER | A 132 | 48.462 | 63.553 | 8.088 | 1.00 | 0.00 | C |
| ATOM | 859 | C | SER | A 132 | 48.604 | 64.160 | 9.488 | 1.00 | 0.00 | C |
| ATOM | 860 | O | SER | A 132 | 49.626 | 64.771 | 9.813 | 1.00 | 0.00 | O |
| ATOM | 861 | CB | SER | A 132 | 47.516 | 64.416 | 7.234 | 1.00 | 0.00 | C |
| ATOM | 862 | OG | SER | A 132 | 46.188 | 64.403 | 7.740 | 1.00 | 0.00 | O |
| ATOM | 863 | N | TYR | A 133 | 47.575 | 63.980 | 10.312 | 1.00 | 0.00 | N |
| ATOM | 864 | CA | TYR | A 133 | 47.588 | 64.482 | 11.684 | 1.00 | 0.00 | C |
| ATOM | 865 | C | TYR | A 133 | 48.554 | 63.692 | 12.565 | 1.00 | 0.00 | C |
| ATOM | 866 | O | TYR | A 133 | 49.316 | 64.278 | 13.339 | 1.00 | 0.00 | O |
| ATOM | 867 | CB | TYR | A 133 | 46.192 | 64.396 | 12.305 | 1.00 | 0.00 | C |
| ATOM | 868 | CG | TYR | A 133 | 45.288 | 65.570 | 12.003 | 1.00 | 0.00 | C |
| ATOM | 869 | CD1 | TYR | A 133 | 44.019 | 65.372 | 11.460 | 1.00 | 0.00 | C |
| ATOM | 870 | CD2 | TYR | A 133 | 45.695 | 66.877 | 12.281 | 1.00 | 0.00 | C |
| ATOM | 871 | CE1 | TYR | A 133 | 43.172 | 66.447 | 11.200 | 1.00 | 0.00 | C |
| ATOM | 872 | CE2 | TYR | A 133 | 44.858 | 67.959 | 12.027 | 1.00 | 0.00 | C |
| ATOM | 873 | CZ | TYR | A 133 | 43.600 | 67.738 | 11.488 | 1.00 | 0.00 | C |
| ATOM | 874 | OH | TYR | A 133 | 42.769 | 68.808 | 11.250 | 1.00 | 0.00 | O |
| ATOM | 875 | N | PHE | A 134 | 48.526 | 62.366 | 12.452 | 1.00 | 0.00 | N |
| ATOM | 876 | CA | PHE | A 134 | 49.397 | 61.541 | 13.284 | 1.00 | 0.00 | C |
| ATOM | 877 | C | PHE | A 134 | 50.876 | 61.830 | 13.058 | 1.00 | 0.00 | C |
| ATOM | 878 | O | PHE | A 134 | 51.655 | 61.898 | 14.012 | 1.00 | 0.00 | O |
| ATOM | 879 | CB | PHE | A 134 | 49.143 | 60.050 | 13.054 | 1.00 | 0.00 | C |
| ATOM | 880 | CG | PHE | A 134 | 49.662 | 59.182 | 14.167 | 1.00 | 0.00 | C |
| ATOM | 881 | CD1 | PHE | A 134 | 48.923 | 59.015 | 15.335 | 1.00 | 0.00 | C |
| ATOM | 882 | CD2 | PHE | A 134 | 50.920 | 58.594 | 14.082 | 1.00 | 0.00 | C |
| ATOM | 883 | CE1 | PHE | A 134 | 49.431 | 58.279 | 16.406 | 1.00 | 0.00 | C |
| ATOM | 884 | CE2 | PHE | A 134 | 51.438 | 57.859 | 15.145 | 1.00 | 0.00 | C |
| ATOM | 885 | CZ | PHE | A 134 | 50.691 | 57.701 | 16.312 | 1.00 | 0.00 | C |
| ATOM | 886 | N | ALA | A 135 | 51.264 | 61.991 | 11.796 | 1.00 | 0.00 | N |
| ATOM | 887 | CA | ALA | A 135 | 52.656 | 62.270 | 11.469 | 1.00 | 0.00 | C |
| ATOM | 888 | C | ALA | A 135 | 53.070 | 63.619 | 12.060 | 1.00 | 0.00 | C |
| ATOM | 889 | O | ALA | A 135 | 54.171 | 63.759 | 12.598 | 1.00 | 0.00 | O |
| ATOM | 890 | CB | ALA | A 135 | 52.845 | 62.265 | 9.957 | 1.00 | 0.00 | C |
| ATOM | 891 | N | ARG | A 136 | 52.181 | 64.602 | 11.958 | 1.00 | 0.00 | N |
| ATOM | 892 | CA | ARG | A 136 | 52.417 | 65.945 | 12.489 | 1.00 | 0.00 | C |
| ATOM | 893 | C | ARG | A 136 | 52.660 | 65.867 | 13.998 | 1.00 | 0.00 | C |
| ATOM | 894 | O | ARG | A 136 | 53.548 | 66.527 | 14.540 | 1.00 | 0.00 | O |
| ATOM | 895 | CB | ARG | A 136 | 51.195 | 66.829 | 12.213 | 1.00 | 0.00 | C |
| ATOM | 896 | CG | ARG | A 136 | 51.232 | 68.221 | 12.856 | 1.00 | 0.00 | C |
| ATOM | 897 | CD | ARG | A 136 | 51.964 | 69.245 | 11.990 | 1.00 | 0.00 | C |
| ATOM | 898 | NE | ARG | A 136 | 51.880 | 70.593 | 12.559 | 1.00 | 0.00 | N |
| ATOM | 899 | CZ | ARG | A 136 | 52.508 | 70.966 | 13.668 | 1.00 | 0.00 | C |
| ATOM | 900 | NH1 | ARG | A 136 | 53.269 | 70.095 | 14.318 | 1.00 | 0.00 | N |
| ATOM | 901 | NH2 | ARG | A 136 | 52.367 | 72.198 | 14.139 | 1.00 | 0.00 | N |
| ATOM | 902 | N | PHE | A 137 | 51.858 | 65.044 | 14.665 | 1.00 | 0.00 | N |
| ATOM | 903 | CA | PHE | A 137 | 51.946 | 64.856 | 16.109 | 1.00 | 0.00 | C |
| ATOM | 904 | C | PHE | A 137 | 53.217 | 64.115 | 16.520 | 1.00 | 0.00 | C |
| ATOM | 905 | O | PHE | A 137 | 54.007 | 64.599 | 17.336 | 1.00 | 0.00 | O |
| ATOM | 906 | CB | PHE | A 137 | 50.729 | 64.065 | 16.587 | 1.00 | 0.00 | C |
| ATOM | 907 | CG | PHE | A 137 | 50.707 | 63.817 | 18.063 | 1.00 | 0.00 | C |
| ATOM | 908 | CD1 | PHE | A 137 | 50.395 | 64.844 | 18.948 | 1.00 | 0.00 | C |
| ATOM | 909 | CD2 | PHE | A 137 | 50.998 | 62.555 | 18.572 | 1.00 | 0.00 | C |
| ATOM | 910 | CE1 | PHE | A 137 | 50.369 | 64.619 | 20.319 | 1.00 | 0.00 | C |
| ATOM | 911 | CE2 | PHE | A 137 | 50.975 | 62.319 | 19.947 | 1.00 | 0.00 | C |
| ATOM | 912 | CZ | PHE | A 137 | 50.659 | 63.356 | 20.819 | 1.00 | 0.00 | C |
| ATOM | 913 | N | TYR | A 138 | 53.400 | 62.933 | 15.943 | 1.00 | 0.00 | N |
| ATOM | 914 | CA | TYR | A 138 | 54.546 | 62.088 | 16.241 | 1.00 | 0.00 | C |
| ATOM | 915 | C | TYR | A 138 | 55.897 | 62.793 | 16.136 | 1.00 | 0.00 | C |
| ATOM | 916 | O | TYR | A 138 | 56.738 | 62.665 | 17.028 | 1.00 | 0.00 | O |
| ATOM | 917 | CB | TYR | A 138 | 54.549 | 60.871 | 15.320 | 1.00 | 0.00 | C |
| ATOM | 918 | CG | TYR | A 138 | 55.576 | 59.832 | 15.706 | 1.00 | 0.00 | C |
| ATOM | 919 | CD1 | TYR | A 138 | 55.322 | 58.928 | 16.734 | 1.00 | 0.00 | C |
| ATOM | 920 | CD2 | TYR | A 138 | 56.800 | 59.752 | 15.043 | 1.00 | 0.00 | C |
| ATOM | 921 | CE1 | TYR | A 138 | 56.261 | 57.964 | 17.091 | 1.00 | 0.00 | C |
| ATOM | 922 | CE2 | TYR | A 138 | 57.748 | 58.789 | 15.395 | 1.00 | 0.00 | C |
| ATOM | 923 | CZ | TYR | A 138 | 57.468 | 57.900 | 16.416 | 1.00 | 0.00 | C |
| ATOM | 924 | OH | TYR | A 138 | 58.383 | 56.930 | 16.757 | 1.00 | 0.00 | O |
| ATOM | 925 | N | HIS | A 139 | 56.117 | 63.523 | 15.047 | 1.00 | 0.00 | N |
| ATOM | 926 | CA | HIS | A 139 | 57.392 | 64.214 | 14.871 | 1.00 | 0.00 | C |
| ATOM | 927 | C | HIS | A 139 | 57.629 | 65.306 | 15.911 | 1.00 | 0.00 | C |
| ATOM | 928 | O | HIS | A 139 | 58.763 | 65.734 | 16.119 | 1.00 | 0.00 | O |
| ATOM | 929 | CB | HIS | A 139 | 57.493 | 64.795 | 13.460 | 1.00 | 0.00 | C |
| ATOM | 930 | CG | HIS | A 139 | 57.671 | 63.756 | 12.395 | 1.00 | 0.00 | C |
| ATOM | 931 | ND1 | HIS | A 139 | 58.778 | 62.935 | 12.335 | 1.00 | 0.00 | N |
| ATOM | 932 | CD2 | HIS | A 139 | 56.880 | 63.398 | 11.355 | 1.00 | 0.00 | C |
| ATOM | 933 | CE1 | HIS | A 139 | 58.661 | 62.118 | 11.302 | 1.00 | 0.00 | C |
| ATOM | 934 | NE2 | HIS | A 139 | 57.519 | 62.378 | 10.691 | 1.00 | 0.00 | N |
| ATOM | 935 | N | ASP | A 140 | 56.560 | 65.751 | 16.563 | 1.00 | 0.00 | N |
| ATOM | 936 | CA | ASP | A 140 | 56.663 | 66.780 | 17.594 | 1.00 | 0.00 | C |
| ATOM | 937 | C | ASP | A 140 | 56.880 | 66.170 | 18.983 | 1.00 | 0.00 | C |
| ATOM | 938 | O | ASP | A 140 | 57.237 | 66.873 | 19.929 | 1.00 | 0.00 | O |
| ATOM | 939 | CB | ASP | A 140 | 55.403 | 67.649 | 17.596 | 1.00 | 0.00 | C |
| ATOM | 940 | CG | ASP | A 140 | 55.510 | 68.833 | 16.652 | 1.00 | 0.00 | C |
| ATOM | 941 | OD1 | ASP | A 140 | 56.361 | 68.794 | 15.739 | 1.00 | 0.00 | O |
| ATOM | 942 | OD2 | ASP | A 140 | 54.738 | 69.802 | 16.819 | 1.00 | 0.00 | O |
| ATOM | 943 | N | LEU | A 141 | 56.672 | 64.861 | 19.097 | 1.00 | 0.00 | N |
| ATOM | 944 | CA | LEU | A 141 | 56.847 | 64.160 | 20.368 | 1.00 | 0.00 | C |
| ATOM | 945 | C | LEU | A 141 | 58.305 | 63.962 | 20.751 | 1.00 | 0.00 | C |
| ATOM | 946 | O | LEU | A 141 | 59.176 | 63.851 | 19.892 | 1.00 | 0.00 | O |
| ATOM | 947 | CB | LEU | A 141 | 56.188 | 62.779 | 20.321 | 1.00 | 0.00 | C |
| ATOM | 948 | CG | LEU | A 141 | 54.670 | 62.624 | 20.382 | 1.00 | 0.00 | C |
| ATOM | 949 | CD1 | LEU | A 141 | 54.324 | 61.135 | 20.287 | 1.00 | 0.00 | C |
| ATOM | 950 | CD2 | LEU | A 141 | 54.132 | 63.209 | 21.679 | 1.00 | 0.00 | C |
| ATOM | 951 | N | GLY | A 142 | 58.563 | 63.916 | 22.054 | 1.00 | 0.00 | N |
| ATOM | 952 | CA | GLY | A 142 | 59.913 | 63.685 | 22.528 | 1.00 | 0.00 | C |
| ATOM | 953 | C | GLY | A 142 | 60.196 | 62.205 | 22.351 | 1.00 | 0.00 | C |
| ATOM | 954 | O | GLY | A 142 | 59.265 | 61.411 | 22.206 | 1.00 | 0.00 | O |
| ATOM | 955 | N | GLU | A 143 | 61.469 | 61.825 | 22.370 | 1.00 | 0.00 | N |
| ATOM | 956 | CA | GLU | A 143 | 61.856 | 60.431 | 22.188 | 1.00 | 0.00 | C |
| ATOM | 957 | C | GLU | A 143 | 61.169 | 59.473 | 23.159 | 1.00 | 0.00 | C |
| ATOM | 958 | O | GLU | A 143 | 60.744 | 58.387 | 22.767 | 1.00 | 0.00 | O |
| ATOM | 959 | CB | GLU | A 143 | 63.375 | 60.289 | 22.308 | 1.00 | 0.00 | C |
| ATOM | 960 | CG | GLU | A 143 | 63.914 | 58.936 | 21.867 | 1.00 | 0.00 | C |
| ATOM | 961 | CD | GLU | A 143 | 63.519 | 58.582 | 20.441 | 1.00 | 0.00 | C |
| ATOM | 962 | OE1 | GLU | A 143 | 63.713 | 59.422 | 19.537 | 1.00 | 0.00 | O |
| ATOM | 963 | OE2 | GLU | A 143 | 63.017 | 57.460 | 20.224 | 1.00 | 0.00 | O |
| ATOM | 964 | N | ASN | A 144 | 61.059 | 59.871 | 24.421 | 1.00 | 0.00 | N |
| ATOM | 965 | CA | ASN | A 144 | 60.416 | 59.027 | 25.423 | 1.00 | 0.00 | C |
| ATOM | 966 | C | ASN | A 144 | 58.972 | 58.723 | 25.026 | 1.00 | 0.00 | C |
| ATOM | 967 | O | ASN | A 144 | 58.536 | 57.572 | 25.082 | 1.00 | 0.00 | O |
| ATOM | 968 | CB | ASN | A 144 | 60.456 | 59.711 | 26.797 | 1.00 | 0.00 | C |
| ATOM | 969 | CG | ASN | A 144 | 59.678 | 58.946 | 27.859 | 1.00 | 0.00 | C |
| ATOM | 970 | OD1 | ASN | A 144 | 58.443 | 58.917 | 27.845 | 1.00 | 0.00 | O |
| ATOM | 971 | ND2 | ASN | A 144 | 60.399 | 58.319 | 28.786 | 1.00 | 0.00 | N |
| ATOM | 972 | N | LYS | A 145 | 58.240 | 59.755 | 24.614 | 1.00 | 0.00 | N |
| ATOM | 973 | CA | LYS | A 145 | 56.847 | 59.590 | 24.210 | 1.00 | 0.00 | C |
| ATOM | 974 | C | LYS | A 145 | 56.698 | 58.798 | 22.912 | 1.00 | 0.00 | C |
| ATOM | 975 | O | LYS | A 145 | 55.735 | 58.046 | 22.752 | 1.00 | 0.00 | O |
| ATOM | 976 | CB | LYS | A 145 | 56.168 | 60.958 | 24.078 | 1.00 | 0.00 | C |
| ATOM | 977 | CG | LYS | A 145 | 55.887 | 61.635 | 25.417 | 1.00 | 0.00 | C |
| ATOM | 978 | CD | LYS | A 145 | 54.925 | 60.800 | 26.252 | 1.00 | 0.00 | C |
| ATOM | 979 | CE | LYS | A 145 | 54.648 | 61.430 | 27.613 | 1.00 | 0.00 | C |
| ATOM | 980 | NZ | LYS | A 145 | 55.860 | 61.462 | 28.478 | 1.00 | 0.00 | N |
| ATOM | 981 | N | LYS | A 146 | 57.640 | 58.966 | 21.986 | 1.00 | 0.00 | N |
| ATOM | 982 | CA | LYS | A 146 | 57.589 | 58.225 | 20.728 | 1.00 | 0.00 | C |
| ATOM | 983 | C | LYS | A 146 | 57.650 | 56.734 | 21.036 | 1.00 | 0.00 | C |
| ATOM | 984 | O | LYS | A 146 | 56.967 | 55.935 | 20.401 | 1.00 | 0.00 | O |
| ATOM | 985 | CB | LYS | A 146 | 58.761 | 58.602 | 19.810 | 1.00 | 0.00 | C |
| ATOM | 986 | CG | LYS | A 146 | 58.624 | 59.956 | 19.127 | 1.00 | 0.00 | C |
| ATOM | 987 | CD | LYS | A 146 | 59.760 | 60.201 | 18.141 | 1.00 | 0.00 | C |
| ATOM | 988 | CE | LYS | A 146 | 59.594 | 61.542 | 17.439 | 1.00 | 0.00 | C |
| ATOM | 989 | NZ | LYS | A 146 | 60.713 | 61.808 | 16.502 | 1.00 | 0.00 | N |
| ATOM | 990 | N | LEU | A 147 | 58.470 | 56.369 | 22.019 | 1.00 | 0.00 | N |
| ATOM | 991 | CA | LEU | A 147 | 58.618 | 54.976 | 22.423 | 1.00 | 0.00 | C |
| ATOM | 992 | C | LEU | A 147 | 57.332 | 54.452 | 23.064 | 1.00 | 0.00 | C |
| ATOM | 993 | O | LEU | A 147 | 56.913 | 53.328 | 22.792 | 1.00 | 0.00 | O |
| ATOM | 994 | CB | LEU | A 147 | 59.795 | 54.828 | 23.394 | 1.00 | 0.00 | C |
| ATOM | 995 | CG | LEU | A 147 | 61.174 | 55.065 | 22.765 | 1.00 | 0.00 | C |
| ATOM | 996 | CD1 | LEU | A 147 | 62.267 | 54.955 | 23.819 | 1.00 | 0.00 | C |
| ATOM | 997 | CD2 | LEU | A 147 | 61.404 | 54.043 | 21.659 | 1.00 | 0.00 | C |
| ATOM | 998 | N | GLN | A 148 | 56.707 | 55.258 | 23.917 | 1.00 | 0.00 | N |
| ATOM | 999 | CA | GLN | A 148 | 55.456 | 54.838 | 24.541 | 1.00 | 0.00 | C |
| ATOM | 1000 | C | GLN | A 148 | 54.393 | 54.655 | 23.461 | 1.00 | 0.00 | C |
| ATOM | 1001 | O | GLN | A 148 | 53.578 | 53.734 | 23.525 | 1.00 | 0.00 | O |
| ATOM | 1002 | CB | GLN | A 148 | 54.961 | 55.874 | 25.552 | 1.00 | 0.00 | C |
| ATOM | 1003 | CG | GLN | A 148 | 55.689 | 55.857 | 26.884 | 1.00 | 0.00 | C |
| ATOM | 1004 | CD | GLN | A 148 | 55.020 | 56.743 | 27.922 | 1.00 | 0.00 | C |
| ATOM | 1005 | OE1 | GLN | A 148 | 55.426 | 56.768 | 29.086 | 1.00 | 0.00 | O |
| ATOM | 1006 | NE2 | GLN | A 148 | 53.990 | 57.477 | 27.505 | 1.00 | 0.00 | N |
| ATOM | 1007 | N | MET | A 149 | 54.407 | 55.537 | 22.468 | 1.00 | 0.00 | N |
| ATOM | 1008 | CA | MET | A 149 | 53.434 | 55.467 | 21.385 | 1.00 | 0.00 | C |
| ATOM | 1009 | C | MET | A 149 | 53.634 | 54.196 | 20.565 | 1.00 | 0.00 | C |
| ATOM | 1010 | O | MET | A 149 | 52.673 | 53.508 | 20.232 | 1.00 | 0.00 | O |
| ATOM | 1011 | CB | MET | A 149 | 53.554 | 56.697 | 20.481 | 1.00 | 0.00 | C |
| ATOM | 1012 | CG | MET | A 149 | 52.533 | 56.747 | 19.352 | 1.00 | 0.00 | C |
| ATOM | 1013 | SD | MET | A 149 | 50.823 | 56.818 | 19.930 | 1.00 | 0.00 | S |
| ATOM | 1014 | CE | MET | A 149 | 50.571 | 58.585 | 20.073 | 1.00 | 0.00 | C |
| ATOM | 1015 | N | LYS | A 150 | 54.885 | 53.879 | 20.244 | 1.00 | 0.00 | N |
| ATOM | 1016 | CA | LYS | A 150 | 55.161 | 52.682 | 19.462 | 1.00 | 0.00 | C |
| ATOM | 1017 | C | LYS | A 150 | 54.741 | 51.429 | 20.215 | 1.00 | 0.00 | C |
| ATOM | 1018 | O | LYS | A 150 | 54.314 | 50.450 | 19.606 | 1.00 | 0.00 | O |
| ATOM | 1019 | CB | LYS | A 150 | 56.650 | 52.601 | 19.104 | 1.00 | 0.00 | C |
| ATOM | 1020 | CG | LYS | A 150 | 57.115 | 53.724 | 18.190 | 1.00 | 0.00 | C |
| ATOM | 1021 | CD | LYS | A 150 | 58.623 | 53.714 | 17.990 | 1.00 | 0.00 | C |
| ATOM | 1022 | CE | LYS | A 150 | 59.076 | 52.493 | 17.214 | 1.00 | 0.00 | C |
| ATOM | 1023 | NZ | LYS | A 150 | 60.543 | 52.537 | 16.948 | 1.00 | 0.00 | N |
| ATOM | 1024 | N | SER | A 151 | 54.839 | 51.464 | 21.542 | 1.00 | 0.00 | N |
| ATOM | 1025 | CA | SER | A 151 | 54.474 | 50.305 | 22.346 | 1.00 | 0.00 | C |
| ATOM | 1026 | C | SER | A 151 | 52.969 | 50.036 | 22.373 | 1.00 | 0.00 | C |
| ATOM | 1027 | O | SER | A 151 | 52.548 | 48.883 | 22.271 | 1.00 | 0.00 | O |
| ATOM | 1028 | CB | SER | A 151 | 55.002 | 50.454 | 23.778 | 1.00 | 0.00 | C |
| ATOM | 1029 | OG | SER | A 151 | 54.275 | 51.437 | 24.486 | 1.00 | 0.00 | O |
| ATOM | 1030 | N | ILE | A 152 | 52.150 | 51.079 | 22.505 | 1.00 | 0.00 | N |
| ATOM | 1031 | CA | ILE | A 152 | 50.710 | 50.854 | 22.528 | 1.00 | 0.00 | C |
| ATOM | 1032 | C | ILE | A 152 | 50.178 | 50.469 | 21.147 | 1.00 | 0.00 | C |
| ATOM | 1033 | O | ILE | A 152 | 49.072 | 49.948 | 21.026 | 1.00 | 0.00 | O |
| ATOM | 1034 | CB | ILE | A 152 | 49.924 | 52.074 | 23.075 | 1.00 | 0.00 | C |
| ATOM | 1035 | CG1 | ILE | A 152 | 50.245 | 53.334 | 22.274 | 1.00 | 0.00 | C |
| ATOM | 1036 | CG2 | ILE | A 152 | 50.249 | 52.274 | 24.549 | 1.00 | 0.00 | C |
| ATOM | 1037 | CD1 | ILE | A 152 | 49.359 | 54.507 | 22.645 | 1.00 | 0.00 | C |
| ATOM | 1038 | N | VAL | A 153 | 50.965 | 50.723 | 20.106 | 1.00 | 0.00 | N |
| ATOM | 1039 | CA | VAL | A 153 | 50.562 | 50.343 | 18.753 | 1.00 | 0.00 | C |
| ATOM | 1040 | C | VAL | A 153 | 50.959 | 48.881 | 18.580 | 1.00 | 0.00 | C |
| ATOM | 1041 | O | VAL | A 153 | 50.178 | 48.057 | 18.103 | 1.00 | 0.00 | O |
| ATOM | 1042 | CB | VAL | A 153 | 51.280 | 51.195 | 17.682 | 1.00 | 0.00 | C |
| ATOM | 1043 | CG1 | VAL | A 153 | 51.068 | 50.590 | 16.295 | 1.00 | 0.00 | C |
| ATOM | 1044 | CG2 | VAL | A 153 | 50.746 | 52.618 | 17.715 | 1.00 | 0.00 | C |
| ATOM | 1045 | N | LYS | A 154 | 52.181 | 48.564 | 18.998 | 1.00 | 0.00 | N |
| ATOM | 1046 | CA | LYS | A 154 | 52.692 | 47.203 | 18.897 | 1.00 | 0.00 | C |
| ATOM | 1047 | C | LYS | A 154 | 51.832 | 46.218 | 19.692 | 1.00 | 0.00 | C |
| ATOM | 1048 | O | LYS | A 154 | 51.604 | 45.088 | 19.244 | 1.00 | 0.00 | O |
| ATOM | 1049 | CB | LYS | A 154 | 54.141 | 47.153 | 19.395 | 1.00 | 0.00 | C |
| ATOM | 1050 | CG | LYS | A 154 | 54.883 | 45.879 | 19.009 | 1.00 | 0.00 | C |
| ATOM | 1051 | CD | LYS | A 154 | 56.371 | 45.971 | 19.327 | 1.00 | 0.00 | C |
| ATOM | 1052 | CE | LYS | A 154 | 56.650 | 45.772 | 20.811 | 1.00 | 0.00 | C |
| ATOM | 1053 | NZ | LYS | A 154 | 55.949 | 46.755 | 21.689 | 1.00 | 0.00 | N |
| ATOM | 1054 | N | ASN | A 155 | 51.344 | 46.643 | 20.857 | 1.00 | 0.00 | N |
| ATOM | 1055 | CA | ASN | A 155 | 50.524 | 45.773 | 21.697 | 1.00 | 0.00 | C |
| ATOM | 1056 | C | ASN | A 155 | 49.050 | 45.705 | 21.292 | 1.00 | 0.00 | C |
| ATOM | 1057 | O | ASN | A 155 | 48.269 | 44.985 | 21.908 | 1.00 | 0.00 | O |
| ATOM | 1058 | CB | ASN | A 155 | 50.637 | 46.177 | 23.178 | 1.00 | 0.00 | C |
| ATOM | 1059 | CG | ASN | A 155 | 49.837 | 47.427 | 23.519 | 1.00 | 0.00 | C |
| ATOM | 1060 | OD1 | ASN | A 155 | 49.102 | 47.957 | 22.688 | 1.00 | 0.00 | O |
| ATOM | 1061 | ND2 | ASN | A 155 | 49.973 | 47.898 | 24.757 | 1.00 | 0.00 | N |
| ATOM | 1062 | N | GLY | A 156 | 48.665 | 46.467 | 20.272 | 1.00 | 0.00 | N |
| ATOM | 1063 | CA | GLY | A 156 | 47.291 | 46.417 | 19.798 | 1.00 | 0.00 | C |
| ATOM | 1064 | C | GLY | A 156 | 46.253 | 47.362 | 20.380 | 1.00 | 0.00 | C |
| ATOM | 1065 | O | GLY | A 156 | 45.080 | 47.277 | 20.006 | 1.00 | 0.00 | O |
| ATOM | 1066 | N | GLN | A 157 | 46.652 | 48.259 | 21.279 | 1.00 | 0.00 | N |
| ATOM | 1067 | CA | GLN | A 157 | 45.695 | 49.195 | 21.872 | 1.00 | 0.00 | C |
| ATOM | 1068 | C | GLN | A 157 | 45.308 | 50.318 | 20.916 | 1.00 | 0.00 | C |
| ATOM | 1069 | O | GLN | A 157 | 44.143 | 50.692 | 20.829 | 1.00 | 0.00 | O |
| ATOM | 1070 | CB | GLN | A 157 | 46.254 | 49.811 | 23.147 | 1.00 | 0.00 | C |
| ATOM | 1071 | CG | GLN | A 157 | 46.313 | 48.867 | 24.328 | 1.00 | 0.00 | C |
| ATOM | 1072 | CD | GLN | A 157 | 46.577 | 49.619 | 25.611 | 1.00 | 0.00 | C |
| ATOM | 1073 | OE1 | GLN | A 157 | 45.676 | 50.244 | 26.173 | 1.00 | 0.00 | O |
| ATOM | 1074 | NE2 | GLN | A 157 | 47.818 | 49.589 | 26.065 | 1.00 | 0.00 | N |
| ATOM | 1075 | N | LEU | A 158 | 46.299 | 50.884 | 20.236 | 1.00 | 0.00 | N |
| ATOM | 1076 | CA | LEU | A 158 | 46.047 | 51.938 | 19.266 | 1.00 | 0.00 | C |
| ATOM | 1077 | C | LEU | A 158 | 46.230 | 51.277 | 17.909 | 1.00 | 0.00 | C |
| ATOM | 1078 | O | LEU | A 158 | 47.308 | 50.772 | 17.598 | 1.00 | 0.00 | O |
| ATOM | 1079 | CB | LEU | A 158 | 47.042 | 53.093 | 19.440 | 1.00 | 0.00 | C |
| ATOM | 1080 | CG | LEU | A 158 | 46.941 | 54.281 | 18.471 | 1.00 | 0.00 | C |
| ATOM | 1081 | CD1 | LEU | A 158 | 47.444 | 53.885 | 17.092 | 1.00 | 0.00 | C |
| ATOM | 1082 | CD2 | LEU | A 158 | 45.508 | 54.766 | 18.392 | 1.00 | 0.00 | C |
| ATOM | 1083 | N | GLU | A 159 | 45.171 | 51.274 | 17.107 | 1.00 | 0.00 | N |
| ATOM | 1084 | CA | GLU | A 159 | 45.230 | 50.638 | 15.801 | 1.00 | 0.00 | C |
| ATOM | 1085 | C | GLU | A 159 | 44.890 | 51.595 | 14.670 | 1.00 | 0.00 | C |
| ATOM | 1086 | O | GLU | A 159 | 43.912 | 52.336 | 14.739 | 1.00 | 0.00 | O |
| ATOM | 1087 | CB | GLU | A 159 | 44.277 | 49.442 | 15.771 | 1.00 | 0.00 | C |
| ATOM | 1088 | CG | GLU | A 159 | 44.210 | 48.721 | 14.434 | 1.00 | 0.00 | C |
| ATOM | 1089 | CD | GLU | A 159 | 43.268 | 47.534 | 14.476 | 1.00 | 0.00 | C |
| ATOM | 1090 | OE1 | GLU | A 159 | 43.603 | 46.536 | 15.148 | 1.00 | 0.00 | O |
| ATOM | 1091 | OE2 | GLU | A 159 | 42.191 | 47.604 | 13.844 | 1.00 | 0.00 | O |
| ATOM | 1092 | N | PHE | A 160 | 45.709 | 51.577 | 13.629 | 1.00 | 0.00 | N |
| ATOM | 1093 | CA | PHE | A 160 | 45.466 | 52.437 | 12.488 | 1.00 | 0.00 | C |
| ATOM | 1094 | C | PHE | A 160 | 44.548 | 51.735 | 11.509 | 1.00 | 0.00 | C |
| ATOM | 1095 | O | PHE | A 160 | 44.727 | 50.552 | 11.202 | 1.00 | 0.00 | O |
| ATOM | 1096 | CB | PHE | A 160 | 46.781 | 52.812 | 11.808 | 1.00 | 0.00 | C |
| ATOM | 1097 | CG | PHE | A 160 | 47.707 | 53.593 | 12.692 | 1.00 | 0.00 | C |
| ATOM | 1098 | CD1 | PHE | A 160 | 48.747 | 52.959 | 13.367 | 1.00 | 0.00 | C |
| ATOM | 1099 | CD2 | PHE | A 160 | 47.508 | 54.956 | 12.892 | 1.00 | 0.00 | C |
| ATOM | 1100 | CE1 | PHE | A 160 | 49.574 | 53.671 | 14.231 | 1.00 | 0.00 | C |
| ATOM | 1101 | CE2 | PHE | A 160 | 48.331 | 55.677 | 13.757 | 1.00 | 0.00 | C |
| ATOM | 1102 | CZ | PHE | A 160 | 49.362 | 55.032 | 14.425 | 1.00 | 0.00 | C |
| ATOM | 1103 | N | VAL | A 161 | 43.543 | 52.468 | 11.048 | 1.00 | 0.00 | N |
| ATOM | 1104 | CA | VAL | A 161 | 42.591 | 51.942 | 10.087 | 1.00 | 0.00 | C |
| ATOM | 1105 | C | VAL | A 161 | 42.805 | 52.718 | 8.791 | 1.00 | 0.00 | C |
| ATOM | 1106 | O | VAL | A 161 | 42.882 | 53.952 | 8.800 | 1.00 | 0.00 | O |
| ATOM | 1107 | CB | VAL | A 161 | 41.138 | 52.085 | 10.605 | 1.00 | 0.00 | C |
| ATOM | 1108 | CG1 | VAL | A 161 | 40.919 | 51.127 | 11.776 | 1.00 | 0.00 | C |
| ATOM | 1109 | CG2 | VAL | A 161 | 40.870 | 53.519 | 11.058 | 1.00 | 0.00 | C |
| ATOM | 1110 | N | THR | A 162 | 42.914 | 51.976 | 7.690 | 1.00 | 0.00 | N |
| ATOM | 1111 | CA | THR | A 162 | 43.179 | 52.519 | 6.358 | 1.00 | 0.00 | C |
| ATOM | 1112 | C | THR | A 162 | 44.641 | 52.973 | 6.314 | 1.00 | 0.00 | C |
| ATOM | 1113 | O | THR | A 162 | 45.451 | 52.417 | 5.572 | 1.00 | 0.00 | O |
| ATOM | 1114 | CB | THR | A 162 | 42.268 | 53.717 | 5.993 | 1.00 | 0.00 | C |
| ATOM | 1115 | OG1 | THR | A 162 | 40.894 | 53.310 | 6.017 | 1.00 | 0.00 | O |
| ATOM | 1116 | CG2 | THR | A 162 | 42.598 | 54.212 | 4.583 | 1.00 | 0.00 | C |
| ATOM | 1117 | N | GLY | A 163 | 44.978 | 53.978 | 7.113 | 1.00 | 0.00 | N |
| ATOM | 1118 | CA | GLY | A 163 | 46.351 | 54.451 | 7.147 | 1.00 | 0.00 | C |
| ATOM | 1119 | C | GLY | A 163 | 46.721 | 55.412 | 6.034 | 1.00 | 0.00 | C |
| ATOM | 1120 | O | GLY | A 163 | 47.895 | 55.562 | 5.705 | 1.00 | 0.00 | O |
| ATOM | 1121 | N | GLY | A 164 | 45.718 | 56.053 | 5.446 | 1.00 | 0.00 | N |
| ATOM | 1122 | CA | GLY | A 164 | 45.980 | 57.014 | 4.393 | 1.00 | 0.00 | C |
| ATOM | 1123 | C | GLY | A 164 | 46.271 | 58.380 | 4.989 | 1.00 | 0.00 | C |
| ATOM | 1124 | O | GLY | A 164 | 45.987 | 58.630 | 6.163 | 1.00 | 0.00 | O |
| ATOM | 1125 | N | TRP | A 165 | 46.847 | 59.267 | 4.186 | 1.00 | 0.00 | N |
| ATOM | 1126 | CA | TRP | A 165 | 47.158 | 60.612 | 4.656 | 1.00 | 0.00 | C |
| ATOM | 1127 | C | TRP | A 165 | 45.848 | 61.228 | 5.155 | 1.00 | 0.00 | C |
| ATOM | 1128 | O | TRP | A 165 | 45.825 | 61.957 | 6.150 | 1.00 | 0.00 | O |
| ATOM | 1129 | CB | TRP | A 165 | 47.739 | 61.440 | 3.507 | 1.00 | 0.00 | C |
| ATOM | 1130 | CG | TRP | A 165 | 48.530 | 62.645 | 3.938 | 1.00 | 0.00 | C |
| ATOM | 1131 | CD1 | TRP | A 165 | 48.290 | 63.948 | 3.600 | 1.00 | 0.00 | C |
| ATOM | 1132 | CD2 | TRP | A 165 | 49.717 | 62.654 | 4.745 | 1.00 | 0.00 | C |
| ATOM | 1133 | NE1 | TRP | A 165 | 49.253 | 64.766 | 4.145 | 1.00 | 0.00 | N |
| ATOM | 1134 | CE2 | TRP | A 165 | 50.141 | 64.000 | 4.851 | 1.00 | 0.00 | C |
| ATOM | 1135 | CE3 | TRP | A 165 | 50.464 | 61.657 | 5.385 | 1.00 | 0.00 | C |
| ATOM | 1136 | CZ2 | TRP | A 165 | 51.282 | 64.375 | 5.575 | 1.00 | 0.00 | C |
| ATOM | 1137 | CZ3 | TRP | A 165 | 51.602 | 62.033 | 6.107 | 1.00 | 0.00 | C |
| ATOM | 1138 | CH2 | TRP | A 165 | 51.995 | 63.380 | 6.193 | 1.00 | 0.00 | C |
| ATOM | 1139 | N | VAL | A 166 | 44.756 | 60.904 | 4.467 | 1.00 | 0.00 | N |
| ATOM | 1140 | CA | VAL | A 166 | 43.425 | 61.402 | 4.822 | 1.00 | 0.00 | C |
| ATOM | 1141 | C | VAL | A 166 | 42.395 | 60.293 | 4.591 | 1.00 | 0.00 | C |
| ATOM | 1142 | O | VAL | A 166 | 42.755 | 59.158 | 4.290 | 1.00 | 0.00 | O |
| ATOM | 1143 | CB | VAL | A 166 | 43.015 | 62.614 | 3.930 | 1.00 | 0.00 | C |
| ATOM | 1144 | CG1 | VAL | A 166 | 44.058 | 63.731 | 4.035 | 1.00 | 0.00 | C |
| ATOM | 1145 | CG2 | VAL | A 166 | 42.873 | 62.167 | 2.465 | 1.00 | 0.00 | C |
| ATOM | 1146 | N | MET | A 167 | 41.122 | 60.635 | 4.773 | 1.00 | 0.00 | N |
| ATOM | 1147 | CA | MET | A 167 | 39.997 | 59.736 | 4.499 | 1.00 | 0.00 | C |
| ATOM | 1148 | C | MET | A 167 | 39.453 | 60.495 | 3.296 | 1.00 | 0.00 | C |
| ATOM | 1149 | O | MET | A 167 | 38.657 | 61.426 | 3.438 | 1.00 | 0.00 | O |
| ATOM | 1150 | CB | MET | A 167 | 38.981 | 59.749 | 5.641 | 1.00 | 0.00 | C |
| ATOM | 1151 | CG | MET | A 167 | 37.730 | 58.923 | 5.362 | 1.00 | 0.00 | C |
| ATOM | 1152 | SD | MET | A 167 | 36.561 | 58.986 | 6.731 | 1.00 | 0.00 | S |
| ATOM | 1153 | CE | MET | A 167 | 37.551 | 58.164 | 8.032 | 1.00 | 0.00 | C |
| ATOM | 1154 | N | PRO | A 168 | 39.877 | 60.103 | 2.085 | 1.00 | 0.00 | N |
| ATOM | 1155 | CA | PRO | A 168 | 39.448 | 60.779 | 0.862 | 1.00 | 0.00 | C |
| ATOM | 1156 | C | PRO | A 168 | 38.006 | 60.758 | 0.399 | 1.00 | 0.00 | C |
| ATOM | 1157 | O | PRO | A 168 | 37.234 | 59.849 | 0.701 | 1.00 | 0.00 | O |
| ATOM | 1158 | CB | PRO | A 168 | 40.367 | 60.175 | −0.193 | 1.00 | 0.00 | C |
| ATOM | 1159 | CG | PRO | A 168 | 40.431 | 58.739 | 0.248 | 1.00 | 0.00 | C |
| ATOM | 1160 | CD PRO | A 168 | 40.629 | 58.872 | 1.763 | 1.00 | 0.00 | C | |
| ATOM | 1161 | N | ASP | A 169 | 37.672 | 61.800 | −0.353 | 1.00 | 0.00 | N |
| ATOM | 1162 | CA | ASP | A 169 | 36.378 | 61.911 | −0.987 | 1.00 | 0.00 | C |
| ATOM | 1163 | C | ASP | A 169 | 36.468 | 60.745 | −1.965 | 1.00 | 0.00 | C |
| ATOM | 1164 | O | ASP | A 169 | 37.569 | 60.379 | −2.382 | 1.00 | 0.00 | O |
| ATOM | 1165 | CB | ASP | A 169 | 36.285 | 63.228 | −1.758 | 1.00 | 0.00 | C |
| ATOM | 1166 | CG | ASP | A 169 | 35.167 | 63.227 | −2.780 | 1.00 | 0.00 | C |
| ATOM | 1167 | OD1 | ASP | A 169 | 34.076 | 62.709 | −2.465 | 1.00 | 0.00 | O |
| ATOM | 1168 | OD2 | ASP | A 169 | 35.374 | 63.756 | −3.890 | 1.00 | 0.00 | O |
| ATOM | 1169 | N | GLU | A 170 | 35.336 | 60.152 | −2.324 | 1.00 | 0.00 | N |
| ATOM | 1170 | CA | GLU | A 170 | 35.367 | 59.028 | −3.250 | 1.00 | 0.00 | C |
| ATOM | 1171 | C | GLU | A 170 | 34.658 | 59.327 | −4.566 | 1.00 | 0.00 | C |
| ATOM | 1172 | O | GLU | A 170 | 34.630 | 58.489 | −5.467 | 1.00 | 0.00 | O |
| ATOM | 1173 | CB | GLU | A 170 | 34.768 | 57.782 | −2.575 | 1.00 | 0.00 | C |
| ATOM | 1174 | CG | GLU | A 170 | 35.635 | 57.268 | −1.417 | 1.00 | 0.00 | C |
| ATOM | 1175 | CD | GLU | A 170 | 35.037 | 56.070 | −0.687 | 1.00 | 0.00 | C |
| ATOM | 1176 | OE1 | GLU | A 170 | 34.240 | 55.330 | −1.301 | 1.00 | 0.00 | O |
| ATOM | 1177 | OE2 | GLU | A 170 | 35.387 | 55.860 | 0.499 | 1.00 | 0.00 | O |
| ATOM | 1178 | N | ALA | A 171 | 34.112 | 60.533 | −4.687 | 1.00 | 0.00 | N |
| ATOM | 1179 | CA | ALA | A 171 | 33.397 | 60.918 | −5.901 | 1.00 | 0.00 | C |
| ATOM | 1180 | C | ALA | A 171 | 34.233 | 61.694 | −6.920 | 1.00 | 0.00 | C |
| ATOM | 1181 | O | ALA | A 171 | 34.279 | 61.342 | −8.099 | 1.00 | 0.00 | O |
| ATOM | 1182 | CB | ALA | A 171 | 32.164 | 61.733 | −5.533 | 1.00 | 0.00 | C |
| ATOM | 1183 | N | ASN | A 172 | 34.888 | 62.752 | −6.452 | 1.00 | 0.00 | N |
| ATOM | 1184 | CA | ASN | A 172 | 35.685 | 63.629 | −7.305 | 1.00 | 0.00 | C |
| ATOM | 1185 | C | ASN | A 172 | 37.145 | 63.220 | −7.465 | 1.00 | 0.00 | C |
| ATOM | 1186 | O | ASN | A 172 | 37.806 | 63.594 | −8.432 | 1.00 | 0.00 | O |
| ATOM | 1187 | CB | ASN | A 172 | 35.643 | 65.045 | −6.733 | 1.00 | 0.00 | C |
| ATOM | 1188 | CG | ASN | A 172 | 34.231 | 65.585 | −6.613 | 1.00 | 0.00 | C |
| ATOM | 1189 | OD1 | ASN | A 172 | 33.572 | 65.854 | −7.617 | 1.00 | 0.00 | O |
| ATOM | 1190 | ND2 | ASN | A 172 | 33.760 | 65.746 | −5.379 | 1.00 | 0.00 | N |
| ATOM | 1191 | N | SER | A 173 | 37.643 | 62.455 | −6.508 | 1.00 | 0.00 | N |
| ATOM | 1192 | CA | SER | A 173 | 39.040 | 62.031 | −6.510 | 1.00 | 0.00 | C |
| ATOM | 1193 | C | SER | A 173 | 39.432 | 61.111 | −7.660 | 1.00 | 0.00 | C |
| ATOM | 1194 | O | SER | A 173 | 38.671 | 60.220 | −8.040 | 1.00 | 0.00 | O |
| ATOM | 1195 | CB | SER | A 173 | 39.349 | 61.327 | −5.193 | 1.00 | 0.00 | C |
| ATOM | 1196 | OG | SER | A 173 | 40.734 | 61.020 | −5.115 | 1.00 | 0.00 | O |
| ATOM | 1197 | N | HIS | A 174 | 40.623 | 61.335 | −8.215 | 1.00 | 0.00 | N |
| ATOM | 1198 | CA | HIS | A 174 | 41.116 | 60.474 | −9.282 | 1.00 | 0.00 | C |
| ATOM | 1199 | C | HIS | A 174 | 41.786 | 59.293 | −8.576 | 1.00 | 0.00 | C |
| ATOM | 1200 | O | HIS | A 174 | 42.429 | 59.480 | −7.545 | 1.00 | 0.00 | O |
| ATOM | 1201 | CB | HIS | A 174 | 42.127 | 61.214 | −10.152 | 1.00 | 0.00 | C |
| ATOM | 1202 | CG | HIS | A 174 | 42.377 | 60.548 | −11.466 | 1.00 | 0.00 | C |
| ATOM | 1203 | ND1 | HIS | A 174 | 43.060 | 59.355 | −11.576 | 1.00 | 0.00 | N |
| ATOM | 1204 | CD2 | HIS | A 174 | 41.972 | 60.869 | −12.717 | 1.00 | 0.00 | C |
| ATOM | 1205 | CE1 | HIS | A 174 | 43.060 | 58.969 | −12.840 | 1.00 | 0.00 | C |
| ATOM | 1206 | NE2 | HIS | A 174 | 42.406 | 59.870 | −13.552 | 1.00 | 0.00 | N |
| ATOM | 1207 | N | TRP | A 175 | 41.642 | 58.085 | −9.115 | 1.00 | 0.00 | N |
| ATOM | 1208 | CA | TRP | A 175 | 42.230 | 56.920 | −8.461 | 1.00 | 0.00 | C |
| ATOM | 1209 | C | TRP | A 175 | 43.722 | 57.085 | −8.224 | 1.00 | 0.00 | C |
| ATOM | 1210 | O | TRP | A 175 | 44.254 | 56.588 | −7.233 | 1.00 | 0.00 | O |
| ATOM | 1211 | CB | TRP | A 175 | 41.964 | 55.628 | −9.254 | 1.00 | 0.00 | C |
| ATOM | 1212 | CG | TRP | A 175 | 42.795 | 55.448 | −10.501 | 1.00 | 0.00 | C |
| ATOM | 1213 | CD1 | TRP | A 175 | 42.468 | 55.823 | −11.774 | 1.00 | 0.00 | C |
| ATOM | 1214 | CD2 | TRP | A 175 | 44.088 | 54.834 | −10.583 | 1.00 | 0.00 | C |
| ATOM | 1215 | NE1 | TRP | A 175 | 43.479 | 55.478 | −12.646 | 1.00 | 0.00 | N |
| ATOM | 1216 | CE2 | TRP | A 175 | 44.485 | 54.870 | −11.939 | 1.00 | 0.00 | C |
| ATOM | 1217 | CE3 | TRP | A 175 | 44.951 | 54.258 | −9.638 | 1.00 | 0.00 | C |
| ATOM | 1218 | CZ2 | TRP | A 175 | 45.711 | 54.348 | −12.378 | 1.00 | 0.00 | C |
| ATOM | 1219 | CZ3 | TRP | A 175 | 46.170 | 53.739 | −10.072 | 1.00 | 0.00 | C |
| ATOM | 1220 | CH2 | TRP | A 175 | 46.537 | 53.789 | −11.434 | 1.00 | 0.00 | C |
| ATOM | 1221 | N | ARG | A 176 | 44.399 | 57.787 | −9.126 | 1.00 | 0.00 | N |
| ATOM | 1222 | CA | ARG | A 176 | 45.830 | 58.000 | −8.974 | 1.00 | 0.00 | C |
| ATOM | 1223 | C | ARG | A 176 | 46.145 | 58.754 | −7.679 | 1.00 | 0.00 | C |
| ATOM | 1224 | O | ARG | A 176 | 47.118 | 58.433 | −6.987 | 1.00 | 0.00 | O |
| ATOM | 1225 | CB | ARG | A 176 | 46.374 | 58.756 | −10.191 | 1.00 | 0.00 | C |
| ATOM | 1226 | CG | ARG | A 176 | 46.436 | 57.886 | −11.445 | 1.00 | 0.00 | C |
| ATOM | 1227 | CD | ARG | A 176 | 46.407 | 58.704 | −12.723 | 1.00 | 0.00 | C |
| ATOM | 1228 | NE | ARG | A 176 | 47.504 | 59.663 | −12.820 | 1.00 | 0.00 | N |
| ATOM | 1229 | CZ | ARG | A 176 | 47.662 | 60.498 | −13.845 | 1.00 | 0.00 | C |
| ATOM | 1230 | NH1 | ARG | A 176 | 46.794 | 60.483 | −14.852 | 1.00 | 0.00 | N |
| ATOM | 1231 | NH2 | ARG | A 176 | 48.677 | 61.349 | −13.861 | 1.00 | 0.00 | N |
| ATOM | 1232 | N | ASN | A 177 | 45.325 | 59.747 | −7.341 | 1.00 | 0.00 | N |
| ATOM | 1233 | CA | ASN | A 177 | 45.557 | 60.511 | −6.116 | 1.00 | 0.00 | C |
| ATOM | 1234 | C | ASN | A 177 | 45.077 | 59.759 | −4.877 | 1.00 | 0.00 | C |
| ATOM | 1235 | O | ASN | A 177 | 45.587 | 59.975 | −3.773 | 1.00 | 0.00 | O |
| ATOM | 1236 | CB | ASN | A 177 | 44.894 | 61.891 | −6.200 | 1.00 | 0.00 | C |
| ATOM | 1237 | CG | ASN | A 177 | 45.567 | 62.787 | −7.220 | 1.00 | 0.00 | C |
| ATOM | 1238 | OD1 | ASN | A 177 | 46.750 | 62.621 | −7.515 | 1.00 | 0.00 | O |
| ATOM | 1239 | ND2 | ASN | A 177 | 44.821 | 63.747 | −7.758 | 1.00 | 0.00 | N |
| ATOM | 1240 | N | VAL | A 178 | 44.095 | 58.882 | −5.055 | 1.00 | 0.00 | N |
| ATOM | 1241 | CA | VAL | A 178 | 43.618 | 58.078 | −3.935 | 1.00 | 0.00 | C |
| ATOM | 1242 | C | VAL | A 178 | 44.797 | 57.175 | −3.558 | 1.00 | 0.00 | C |
| ATOM | 1243 | O | VAL | A 178 | 45.095 | 56.980 | −2.378 | 1.00 | 0.00 | O |
| ATOM | 1244 | CB | VAL | A 178 | 42.404 | 57.198 | −4.336 | 1.00 | 0.00 | C |
| ATOM | 1245 | CG1 | VAL | A 178 | 42.074 | 56.207 | −3.215 | 1.00 | 0.00 | C |
| ATOM | 1246 | CG2 | VAL | A 178 | 41.199 | 58.079 | −4.631 | 1.00 | 0.00 | C |
| ATOM | 1247 | N | LEU | A 179 | 45.476 | 56.634 | −4.570 | 1.00 | 0.00 | N |
| ATOM | 1248 | CA | LEU | A 179 | 46.629 | 55.768 | −4.326 | 1.00 | 0.00 | C |
| ATOM | 1249 | C | LEU | A 179 | 47.783 | 56.568 | −3.739 | 1.00 | 0.00 | C |
| ATOM | 1250 | O | LEU | A 179 | 48.484 | 56.096 | −2.839 | 1.00 | 0.00 | O |
| ATOM | 1251 | CB | LEU | A 179 | 47.098 | 55.093 | −5.622 | 1.00 | 0.00 | C |
| ATOM | 1252 | CG | LEU | A 179 | 48.400 | 54.279 | −5.498 | 1.00 | 0.00 | C |
| ATOM | 1253 | CD1 | LEU | A 179 | 48.236 | 53.163 | −4.463 | 1.00 | 0.00 | C |
| ATOM | 1254 | CD2 | LEU | A 179 | 48.759 | 53.699 | −6.860 | 1.00 | 0.00 | C |
| ATOM | 1255 | N | LEU | A 180 | 47.980 | 57.782 | −4.250 | 1.00 | 0.00 | N |
| ATOM | 1256 | CA | LEU | A 180 | 49.060 | 58.635 | −3.763 | 1.00 | 0.00 | C |
| ATOM | 1257 | C | LEU | A 180 | 48.922 | 58.898 | −2.261 | 1.00 | 0.00 | C |
| ATOM | 1258 | O | LEU | A 180 | 49.876 | 58.707 | −1.499 | 1.00 | 0.00 | O |
| ATOM | 1259 | CB | LEU | A 180 | 49.066 | 59.970 | −4.521 | 1.00 | 0.00 | C |
| ATOM | 1260 | CG | LEU | A 180 | 50.209 | 60.929 | −4.169 | 1.00 | 0.00 | C |
| ATOM | 1261 | CD1 | LEU | A 180 | 51.515 | 60.375 | −4.738 | 1.00 | 0.00 | C |
| ATOM | 1262 | CD2 | LEU | A 180 | 49.928 | 62.322 | −4.727 | 1.00 | 0.00 | C |
| ATOM | 1263 | N | GLN | A 181 | 47.737 | 59.322 | −1.825 | 1.00 | 0.00 | N |
| ATOM | 1264 | CA | GLN | A 181 | 47.544 | 59.619 | −0.409 | 1.00 | 0.00 | C |
| ATOM | 1265 | C | GLN | A 181 | 47.588 | 58.377 | 0.476 | 1.00 | 0.00 | C |
| ATOM | 1266 | O | GLN | A 181 | 48.068 | 58.446 | 1.605 | 1.00 | 0.00 | O |
| ATOM | 1267 | CB | GLN | A 181 | 46.247 | 60.419 | −0.186 | 1.00 | 0.00 | C |
| ATOM | 1268 | CG | GLN | A 181 | 44.931 | 59.680 | −0.425 | 1.00 | 0.00 | C |
| ATOM | 1269 | CD | GLN | A 181 | 44.567 | 58.741 | 0.714 | 1.00 | 0.00 | C |
| ATOM | 1270 | OE1 | GLN | A 181 | 44.821 | 59.037 | 1.887 | 1.00 | 0.00 | O |
| ATOM | 1271 | NE2 | GLN | A 181 | 43.956 | 57.610 | 0.377 | 1.00 | 0.00 | N |
| ATOM | 1272 | N | LEU | A 182 | 47.104 | 57.243 | −0.029 | 1.00 | 0.00 | N |
| ATOM | 1273 | CA | LEU | A 182 | 47.151 | 56.011 | 0.750 | 1.00 | 0.00 | C |
| ATOM | 1274 | C | LEU | A 182 | 48.623 | 55.644 | 0.939 | 1.00 | 0.00 | C |
| ATOM | 1275 | O | LEU | A 182 | 49.058 | 55.307 | 2.040 | 1.00 | 0.00 | O |
| ATOM | 1276 | CB | LEU | A 182 | 46.429 | 54.868 | 0.019 | 1.00 | 0.00 | C |
| ATOM | 1277 | CG | LEU | A 182 | 46.497 | 53.482 | 0.679 | 1.00 | 0.00 | C |
| ATOM | 1278 | CD1 | LEU | A 182 | 45.750 | 53.491 | 2.011 | 1.00 | 0.00 | C |
| ATOM | 1279 | CD2 | LEU | A 182 | 45.891 | 52.437 | −0.261 | 1.00 | 0.00 | C |
| ATOM | 1280 | N | THR | A 183 | 49.389 | 55.735 | −0.145 | 1.00 | 0.00 | N |
| ATOM | 1281 | CA | THR | A 183 | 50.810 | 55.408 | −0.113 | 1.00 | 0.00 | C |
| ATOM | 1282 | C | THR | A 183 | 51.569 | 56.350 | 0.825 | 1.00 | 0.00 | C |
| ATOM | 1283 | O | THR | A 183 | 52.437 | 55.919 | 1.592 | 1.00 | 0.00 | O |
| ATOM | 1284 | CB | THR | A 183 | 51.431 | 55.489 | −1.539 | 1.00 | 0.00 | C |
| ATOM | 1285 | OG1 | THR | A 183 | 50.708 | 54.626 | −2.430 | 1.00 | 0.00 | O |
| ATOM | 1286 | CG2 | THR | A 183 | 52.899 | 55.060 | −1.513 | 1.00 | 0.00 | C |
| ATOM | 1287 | N | GLU | A 184 | 51.238 | 57.636 | 0.775 | 1.00 | 0.00 | N |
| ATOM | 1288 | CA | GLU | A 184 | 51.916 | 58.611 | 1.625 | 1.00 | 0.00 | C |
| ATOM | 1289 | C | GLU | A 184 | 51.715 | 58.250 | 3.100 | 1.00 | 0.00 | C |
| ATOM | 1290 | O | GLU | A 184 | 52.663 | 58.257 | 3.888 | 1.00 | 0.00 | O |
| ATOM | 1291 | CB | GLU | A 184 | 51.376 | 60.017 | 1.342 | 1.00 | 0.00 | C |
| ATOM | 1292 | CG | GLU | A 184 | 52.229 | 61.165 | 1.883 | 1.00 | 0.00 | C |
| ATOM | 1293 | CD | GLU | A 184 | 53.624 | 61.226 | 1.269 | 1.00 | 0.00 | C |
| ATOM | 1294 | OE1 | GLU | A 184 | 53.778 | 60.909 | 0.070 | 1.00 | 0.00 | O |
| ATOM | 1295 | OE2 | GLU | A 184 | 54.567 | 61.615 | 1.988 | 1.00 | 0.00 | O |
| ATOM | 1296 | N | GLY | A 185 | 50.479 | 57.921 | 3.463 | 1.00 | 0.00 | N |
| ATOM | 1297 | CA | GLY | A 185 | 50.182 | 57.560 | 4.840 | 1.00 | 0.00 | C |
| ATOM | 1298 | C | GLY | A 185 | 50.766 | 56.225 | 5.271 | 1.00 | 0.00 | C |
| ATOM | 1299 | O | GLY | A 185 | 51.350 | 56.112 | 6.356 | 1.00 | 0.00 | O |
| ATOM | 1300 | N | GLN | A 186 | 50.623 | 55.204 | 4.432 | 1.00 | 0.00 | N |
| ATOM | 1301 | CA | GLN | A 186 | 51.142 | 53.886 | 4.788 | 1.00 | 0.00 | C |
| ATOM | 1302 | C | GLN | A 186 | 52.666 | 53.803 | 4.781 | 1.00 | 0.00 | C |
| ATOM | 1303 | O | GLN | A 186 | 53.254 | 53.043 | 5.551 | 1.00 | 0.00 | O |
| ATOM | 1304 | CB | GLN | A 186 | 50.559 | 52.808 | 3.870 | 1.00 | 0.00 | C |
| ATOM | 1305 | CG | GLN | A 186 | 49.045 | 52.631 | 4.004 | 1.00 | 0.00 | C |
| ATOM | 1306 | CD | GLN | A 186 | 48.614 | 51.190 | 3.797 | 1.00 | 0.00 | C |
| ATOM | 1307 | OE1 | GLN | A 186 | 49.190 | 50.476 | 2.981 | 1.00 | 0.00 | O |
| ATOM | 1308 | NE2 | GLN | A 186 | 47.592 | 50.758 | 4.531 | 1.00 | 0.00 | N |
| ATOM | 1309 | N | THR | A 187 | 53.312 | 54.574 | 3.916 | 1.00 | 0.00 | N |
| ATOM | 1310 | CA | THR | A 187 | 54.768 | 54.551 | 3.873 | 1.00 | 0.00 | C |
| ATOM | 1311 | C | THR | A 187 | 55.289 | 55.132 | 5.188 | 1.00 | 0.00 | C |
| ATOM | 1312 | O | THR | A 187 | 56.255 | 54.629 | 5.765 | 1.00 | 0.00 | O |
| ATOM | 1313 | CB | THR | A 187 | 55.297 | 55.355 | 2.673 | 1.00 | 0.00 | C |
| ATOM | 1314 | OG1 | THR | A 187 | 54.837 | 54.743 | 1.460 | 1.00 | 0.00 | O |
| ATOM | 1315 | CG2 | THR | A 187 | 56.827 | 55.373 | 2.664 | 1.00 | 0.00 | C |
| ATOM | 1316 | N | TRP | A 188 | 54.631 | 56.179 | 5.675 | 1.00 | 0.00 | N |
| ATOM | 1317 | CA | TRP | A 188 | 55.034 | 56.790 | 6.937 | 1.00 | 0.00 | C |
| ATOM | 1318 | C | TRP | A 188 | 54.838 | 55.761 | 8.052 | 1.00 | 0.00 | C |
| ATOM | 1319 | O | TRP | A 188 | 55.727 | 55.551 | 8.875 | 1.00 | 0.00 | O |
| ATOM | 1320 | CB | TRP | A 188 | 54.189 | 58.033 | 7.239 | 1.00 | 0.00 | C |
| ATOM | 1321 | CG | TRP | A 188 | 54.655 | 58.785 | 8.461 | 1.00 | 0.00 | C |
| ATOM | 1322 | CD1 | TRP | A 188 | 55.574 | 59.798 | 8.503 | 1.00 | 0.00 | C |
| ATOM | 1323 | CD2 | TRP | A 188 | 54.297 | 58.513 | 9.823 | 1.00 | 0.00 | C |
| ATOM | 1324 | NE1 | TRP | A 188 | 55.815 | 60.168 | 9.808 | 1.00 | 0.00 | N |
| ATOM | 1325 | CE2 | TRP | A 188 | 55.044 | 59.395 | 10.637 | 1.00 | 0.00 | C |
| ATOM | 1326 | CE3 | TRP | A 188 | 53.422 | 57.605 | 10.434 | 1.00 | 0.00 | C |
| ATOM | 1327 | CZ2 | TRP | A 188 | 54.942 | 59.394 | 12.032 | 1.00 | 0.00 | C |
| ATOM | 1328 | CZ3 | TRP | A 188 | 53.322 | 57.603 | 11.826 | 1.00 | 0.00 | C |
| ATOM | 1329 | CH2 | TRP | A 188 | 54.079 | 58.492 | 12.607 | 1.00 | 0.00 | C |
| ATOM | 1330 | N | LEU | A 189 | 53.672 | 55.115 | 8.071 | 1.00 | 0.00 | N |
| ATOM | 1331 | CA | LEU | A 189 | 53.375 | 54.113 | 9.094 | 1.00 | 0.00 | C |
| ATOM | 1332 | C | LEU | A 189 | 54.375 | 52.962 | 9.114 | 1.00 | 0.00 | C |
| ATOM | 1333 | O | LEU | A 189 | 54.793 | 52.515 | 10.185 | 1.00 | 0.00 | O |
| ATOM | 1334 | CB | LEU | A 189 | 51.965 | 53.542 | 8.901 | 1.00 | 0.00 | C |
| ATOM | 1335 | CG | LEU | A 189 | 50.798 | 54.436 | 9.325 | 1.00 | 0.00 | C |
| ATOM | 1336 | CD1 | LEU | A 189 | 49.476 | 53.723 | 9.014 | 1.00 | 0.00 | C |
| ATOM | 1337 | CD2 | LEU | A 189 | 50.908 | 54.745 | 10.821 | 1.00 | 0.00 | C |
| ATOM | 1338 | N | LYS | A 190 | 54.753 | 52.470 | 7.939 | 1.00 | 0.00 | N |
| ATOM | 1339 | CA | LYS | A 190 | 55.704 | 51.365 | 7.885 | 1.00 | 0.00 | C |
| ATOM | 1340 | C | LYS | A 190 | 57.047 | 51.789 | 8.466 | 1.00 | 0.00 | C |
| ATOM | 1341 | O | LYS | A 190 | 57.646 | 51.066 | 9.261 | 1.00 | 0.00 | O |
| ATOM | 1342 | CB | LYS | A 190 | 55.913 | 50.883 | 6.446 | 1.00 | 0.00 | C |
| ATOM | 1343 | CG | LYS | A 190 | 56.747 | 49.601 | 6.350 | 1.00 | 0.00 | C |
| ATOM | 1344 | CD | LYS | A 190 | 56.968 | 49.178 | 4.906 | 1.00 | 0.00 | C |
| ATOM | 1345 | CE | LYS | A 190 | 57.527 | 47.761 | 4.815 | 1.00 | 0.00 | C |
| ATOM | 1346 | NZ | LYS | A 190 | 58.685 | 47.571 | 5.732 | 1.00 | 0.00 | N |
| ATOM | 1347 | N | GLN | A 191 | 57.511 | 52.967 | 8.071 | 1.00 | 0.00 | N |
| ATOM | 1348 | CA | GLN | A 191 | 58.791 | 53.471 | 8.542 | 1.00 | 0.00 | C |
| ATOM | 1349 | C | GLN | A 191 | 58.847 | 53.796 | 10.034 | 1.00 | 0.00 | C |
| ATOM | 1350 | O | GLN | A 191 | 59.810 | 53.431 | 10.710 | 1.00 | 0.00 | O |
| ATOM | 1351 | CB | GLN | A 191 | 59.196 | 54.715 | 7.741 | 1.00 | 0.00 | C |
| ATOM | 1352 | CG | GLN | A 191 | 60.522 | 55.317 | 8.190 | 1.00 | 0.00 | C |
| ATOM | 1353 | CD | GLN | A 191 | 60.941 | 56.526 | 7.371 | 1.00 | 0.00 | C |
| ATOM | 1354 | OE1 | GLN | A 191 | 61.982 | 57.132 | 7.635 | 1.00 | 0.00 | O |
| ATOM | 1355 | NE2 | GLN | A 191 | 60.135 | 56.883 | 6.373 | 1.00 | 0.00 | N |
| ATOM | 1356 | N | PHE | A 192 | 57.826 | 54.471 | 10.556 | 1.00 | 0.00 | N |
| ATOM | 1357 | CA | PHE | A 192 | 57.842 | 54.853 | 11.967 | 1.00 | 0.00 | C |
| ATOM | 1358 | C | PHE | A 192 | 57.044 | 54.008 | 12.965 | 1.00 | 0.00 | C |
| ATOM | 1359 | O | PHE | A 192 | 57.394 | 53.969 | 14.146 | 1.00 | 0.00 | O |
| ATOM | 1360 | CB | PHE | A 192 | 57.422 | 56.319 | 12.110 | 1.00 | 0.00 | C |
| ATOM | 1361 | CG | PHE | A 192 | 58.327 | 57.278 | 11.395 | 1.00 | 0.00 | C |
| ATOM | 1362 | CD1 | PHE | A 192 | 58.024 | 57.716 | 10.108 | 1.00 | 0.00 | C |
| ATOM | 1363 | CD2 | PHE | A 192 | 59.497 | 57.727 | 11.997 | 1.00 | 0.00 | C |
| ATOM | 1364 | CE1 | PHE | A 192 | 58.870 | 58.587 | 9.432 | 1.00 | 0.00 | C |
| ATOM | 1365 | CE2 | PHE | A 192 | 60.355 | 58.601 | 11.329 | 1.00 | 0.00 | C |
| ATOM | 1366 | CZ | PHE | A 192 | 60.041 | 59.032 | 10.044 | 1.00 | 0.00 | C |
| ATOM | 1367 | N | MET | A 193 | 55.985 | 53.343 | 12.512 | 1.00 | 0.00 | N |
| ATOM | 1368 | CA | MET | A 193 | 55.165 | 52.521 | 13.410 | 1.00 | 0.00 | C |
| ATOM | 1369 | C | MET | A 193 | 55.333 | 51.025 | 13.145 | 1.00 | 0.00 | C |
| ATOM | 1370 | O | MET | A 193 | 54.859 | 50.190 | 13.920 | 1.00 | 0.00 | O |
| ATOM | 1371 | CB | MET | A 193 | 53.679 | 52.880 | 13.274 | 1.00 | 0.00 | C |
| ATOM | 1372 | CG | MET | A 193 | 53.268 | 54.222 | 13.862 | 1.00 | 0.00 | C |
| ATOM | 1373 | SD | MET | A 193 | 53.639 | 54.388 | 15.631 | 1.00 | 0.00 | S |
| ATOM | 1374 | CE | MET | A 193 | 54.987 | 55.512 | 15.487 | 1.00 | 0.00 | C |
| ATOM | 1375 | N | ASN | A 194 | 55.997 | 50.698 | 12.041 | 1.00 | 0.00 | N |
| ATOM | 1376 | CA | ASN | A 194 | 56.234 | 49.312 | 11.654 | 1.00 | 0.00 | C |
| ATOM | 1377 | C | ASN | A 194 | 54.932 | 48.521 | 11.530 | 1.00 | 0.00 | C |
| ATOM | 1378 | O | ASN | A 194 | 54.839 | 47.377 | 11.975 | 1.00 | 0.00 | O |
| ATOM | 1379 | CB | ASN | A 194 | 57.168 | 48.640 | 12.669 | 1.00 | 0.00 | C |
| ATOM | 1380 | CG | ASN | A 194 | 57.626 | 47.263 | 12.223 | 1.00 | 0.00 | C |
| ATOM | 1381 | OD1 | ASN | A 194 | 57.761 | 47.001 | 11.026 | 1.00 | 0.00 | O |
| ATOM | 1382 | ND2 | ASN | A 194 | 57.880 | 46.389 | 13.194 | 1.00 | 0.00 | N |
| ATOM | 1383 | N | VAL | A 195 | 53.920 | 49.140 | 10.932 | 1.00 | 0.00 | N |
| ATOM | 1384 | CA | VAL | A 195 | 52.633 | 48.478 | 10.741 | 1.00 | 0.00 | C |
| ATOM | 1385 | C | VAL | A 195 | 52.002 | 48.928 | 9.428 | 1.00 | 0.00 | C |
| ATOM | 1386 | O | VAL | A 195 | 52.223 | 50.052 | 8.979 | 1.00 | 0.00 | O |
| ATOM | 1387 | CB | VAL | A 195 | 51.631 | 48.789 | 11.891 | 1.00 | 0.00 | C |
| ATOM | 1388 | CG1 | VAL | A 195 | 52.186 | 48.303 | 13.228 | 1.00 | 0.00 | C |
| ATOM | 1389 | CG2 | VAL | A 195 | 51.334 | 50.282 | 11.941 | 1.00 | 0.00 | C |
| ATOM | 1390 | N | THR | A 196 | 51.230 | 48.032 | 8.821 | 1.00 | 0.00 | N |
| ATOM | 1391 | CA | THR | A 196 | 50.528 | 48.308 | 7.570 | 1.00 | 0.00 | C |
| ATOM | 1392 | C | THR | A 196 | 49.094 | 47.815 | 7.740 | 1.00 | 0.00 | C |
| ATOM | 1393 | O | THR | A 196 | 48.847 | 46.611 | 7.778 | 1.00 | 0.00 | O |
| ATOM | 1394 | CB | THR | A 196 | 51.159 | 47.554 | 6.373 | 1.00 | 0.00 | C |
| ATOM | 1395 | OG1 | THR | A 196 | 52.518 | 47.973 | 6.195 | 1.00 | 0.00 | O |
| ATOM | 1396 | CG2 | THR | A 196 | 50.377 | 47.837 | 5.099 | 1.00 | 0.00 | C |
| ATOM | 1397 | N | PRO | A 197 | 48.130 | 48.743 | 7.852 | 1.00 | 0.00 | N |
| ATOM | 1398 | CA | PRO | A 197 | 46.722 | 48.371 | 8.020 | 1.00 | 0.00 | C |
| ATOM | 1399 | C | PRO | A 197 | 46.182 | 47.503 | 6.889 | 1.00 | 0.00 | C |
| ATOM | 1400 | O | PRO | A 197 | 46.539 | 47.693 | 5.724 | 1.00 | 0.00 | O |
| ATOM | 1401 | CB | PRO | A 197 | 46.013 | 49.722 | 8.078 | 1.00 | 0.00 | C |
| ATOM | 1402 | CG | PRO | A 197 | 47.055 | 50.625 | 8.681 | 1.00 | 0.00 | C |
| ATOM | 1403 | CD | PRO | A 197 | 48.302 | 50.203 | 7.946 | 1.00 | 0.00 | C |
| ATOM | 1404 | N | THR | A 198 | 45.333 | 46.541 | 7.240 | 1.00 | 0.00 | N |
| ATOM | 1405 | CA | THR | A 198 | 44.717 | 45.674 | 6.244 | 1.00 | 0.00 | C |
| ATOM | 1406 | C | THR | A 198 | 43.206 | 45.838 | 6.323 | 1.00 | 0.00 | C |
| ATOM | 1407 | O | THR | A 198 | 42.465 | 45.141 | 5.634 | 1.00 | 0.00 | O |
| ATOM | 1408 | CB | THR | A 198 | 45.064 | 44.185 | 6.454 | 1.00 | 0.00 | C |
| ATOM | 1409 | OG1 | THR | A 198 | 44.607 | 43.763 | 7.741 | 1.00 | 0.00 | O |
| ATOM | 1410 | CG2 | THR | A 198 | 46.567 | 43.964 | 6.330 | 1.00 | 0.00 | C |
| ATOM | 1411 | N | ALA | A 199 | 42.760 | 46.761 | 7.175 | 1.00 | 0.00 | N |
| ATOM | 1412 | CA | ALA | A 199 | 41.337 | 47.054 | 7.332 | 1.00 | 0.00 | C |
| ATOM | 1413 | C | ALA | A 199 | 41.113 | 48.551 | 7.090 | 1.00 | 0.00 | C |
| ATOM | 1414 | O | ALA | A 199 | 41.811 | 49.389 | 7.665 | 1.00 | 0.00 | O |
| ATOM | 1415 | CB | ALA | A 199 | 40.862 | 46.664 | 8.738 | 1.00 | 0.00 | C |
| ATOM | 1416 | N | SER | A 200 | 40.140 | 48.882 | 6.248 | 1.00 | 0.00 | N |
| ATOM | 1417 | CA | SER | A 200 | 39.850 | 50.280 | 5.931 | 1.00 | 0.00 | C |
| ATOM | 1418 | C | SER | A 200 | 38.615 | 50.799 | 6.658 | 1.00 | 0.00 | C |
| ATOM | 1419 | O | SER | A 200 | 37.648 | 50.059 | 6.881 | 1.00 | 0.00 | O |
| ATOM | 1420 | CB | SER | A 200 | 39.676 | 50.458 | 4.421 | 1.00 | 0.00 | C |
| ATOM | 1421 | OG | SER | A 200 | 39.526 | 51.827 | 4.088 | 1.00 | 0.00 | O |
| ATOM | 1422 | N | TRP | A 201 | 38.658 | 52.083 | 7.003 | 1.00 | 0.00 | N |
| ATOM | 1423 | CA | TRP | A 201 | 37.596 | 52.760 | 7.743 | 1.00 | 0.00 | C |
| ATOM | 1424 | C | TRP | A 201 | 37.226 | 54.077 | 7.043 | 1.00 | 0.00 | C |
| ATOM | 1425 | O | TRP | A 201 | 37.994 | 55.036 | 7.063 | 1.00 | 0.00 | O |
| ATOM | 1426 | CB | TRP | A 201 | 38.112 | 52.994 | 9.176 | 1.00 | 0.00 | C |
| ATOM | 1427 | CG | TRP | A 201 | 37.299 | 53.854 | 10.114 | 1.00 | 0.00 | C |
| ATOM | 1428 | CD1 | TRP | A 201 | 37.455 | 55.195 | 10.339 | 1.00 | 0.00 | C |
| ATOM | 1429 | CD2 | TRP | A 201 | 36.311 | 53.406 | 11.051 | 1.00 | 0.00 | C |
| ATOM | 1430 | NE1 | TRP | A 201 | 36.635 | 55.606 | 11.363 | 1.00 | 0.00 | N |
| ATOM | 1431 | CE2 | TRP | A 201 | 35.921 | 54.529 | 11.819 | 1.00 | 0.00 | C |
| ATOM | 1432 | CE3 | TRP | A 201 | 35.719 | 52.163 | 11.320 | 1.00 | 0.00 | C |
| ATOM | 1433 | CZ2 | TRP | A 201 | 34.968 | 54.446 | 12.840 | 1.00 | 0.00 | C |
| ATOM | 1434 | CZ3 | TRP | A 201 | 34.769 | 52.080 | 12.340 | 1.00 | 0.00 | C |
| ATOM | 1435 | CH2 | TRP | A 201 | 34.404 | 53.219 | 13.087 | 1.00 | 0.00 | C |
| ATOM | 1436 | N | ALA | A 202 | 36.055 | 54.104 | 6.410 | 1.00 | 0.00 | N |
| ATOM | 1437 | CA | ALA | A 202 | 35.580 | 55.290 | 5.691 | 1.00 | 0.00 | C |
| ATOM | 1438 | C | ALA | A 202 | 34.155 | 55.616 | 6.137 | 1.00 | 0.00 | C |
| ATOM | 1439 | O | ALA | A 202 | 33.186 | 55.168 | 5.527 | 1.00 | 0.00 | O |
| ATOM | 1440 | CB | ALA | A 202 | 35.619 | 55.034 | 4.185 | 1.00 | 0.00 | C |
| ATOM | 1441 | N | ILE | A 203 | 34.042 | 56.420 | 7.190 | 1.00 | 0.00 | N |
| ATOM | 1442 | CA | ILE | A 203 | 32.749 | 56.772 | 7.770 | 1.00 | 0.00 | C |
| ATOM | 1443 | C | ILE | A 203 | 32.117 | 58.098 | 7.354 | 1.00 | 0.00 | C |
| ATOM | 1444 | O | ILE | A 203 | 30.941 | 58.323 | 7.642 | 1.00 | 0.00 | O |
| ATOM | 1445 | CB | ILE | A 203 | 32.830 | 56.768 | 9.321 | 1.00 | 0.00 | C |
| ATOM | 1446 | CG1 | ILE | A 203 | 33.902 | 57.764 | 9.786 | 1.00 | 0.00 | C |
| ATOM | 1447 | CG2 | ILE | A 203 | 33.146 | 55.351 | 9.830 | 1.00 | 0.00 | C |
| ATOM | 1448 | CD1 | ILE | A 203 | 33.977 | 57.955 | 11.299 | 1.00 | 0.00 | C |
| ATOM | 1449 | N | ASP | A 204 | 32.861 | 58.973 | 6.679 | 1.00 | 0.00 | N |
| ATOM | 1450 | CA | ASP | A 204 | 32.276 | 60.262 | 6.307 | 1.00 | 0.00 | C |
| ATOM | 1451 | C | ASP | A 204 | 32.157 | 60.645 | 4.827 | 1.00 | 0.00 | C |
| ATOM | 1452 | O | ASP | A 204 | 31.416 | 61.575 | 4.503 | 1.00 | 0.00 | O |
| ATOM | 1453 | CB | ASP | A 204 | 32.981 | 61.400 | 7.056 | 1.00 | 0.00 | C |
| ATOM | 1454 | CG | ASP | A 204 | 32.053 | 62.585 | 7.330 | 1.00 | 0.00 | C |
| ATOM | 1455 | OD1 | ASP | A 204 | 32.553 | 63.709 | 7.548 | 1.00 | 0.00 | O |
| ATOM | 1456 | OD2 | ASP | A 204 | 30.819 | 62.398 | 7.344 | 1.00 | 0.00 | O |
| ATOM | 1457 | N | PRO | A 205 | 32.882 | 59.965 | 3.911 | 1.00 | 0.00 | N |
| ATOM | 1458 | CA | PRO | A 205 | 32.733 | 60.362 | 2.501 | 1.00 | 0.00 | C |
| ATOM | 1459 | C | PRO | A 205 | 31.252 | 60.324 | 2.107 | 1.00 | 0.00 | C |
| ATOM | 1460 | O | PRO | A 205 | 30.514 | 59.447 | 2.552 | 1.00 | 0.00 | O |
| ATOM | 1461 | CB | PRO | A 205 | 33.569 | 59.321 | 1.759 | 1.00 | 0.00 | C |
| ATOM | 1462 | CG | PRO | A 205 | 34.661 | 59.016 | 2.749 | 1.00 | 0.00 | C |
| ATOM | 1463 | CD | PRO | A 205 | 33.882 | 58.890 | 4.046 | 1.00 | 0.00 | C |
| ATOM | 1464 | N | PHE | A 206 | 30.826 | 61.265 | 1.266 | 1.00 | 0.00 | N |
| ATOM | 1465 | CA | PHE | A 206 | 29.418 | 61.368 | 0.866 | 1.00 | 0.00 | C |
| ATOM | 1466 | C | PHE | A 206 | 29.066 | 60.457 | −0.311 | 1.00 | 0.00 | C |
| ATOM | 1467 | O | PHE | A 206 | 28.873 | 60.914 | −1.438 | 1.00 | 0.00 | O |
| ATOM | 1468 | CB | PHE | A 206 | 29.099 | 62.830 | 0.533 | 1.00 | 0.00 | C |
| ATOM | 1469 | CG | PHE | A 206 | 29.935 | 63.822 | 1.306 | 1.00 | 0.00 | C |
| ATOM | 1470 | CD1 | PHE | A 206 | 30.173 | 63.645 | 2.668 | 1.00 | 0.00 | C |
| ATOM | 1471 | CD2 | PHE | A 206 | 30.494 | 64.931 | 0.669 | 1.00 | 0.00 | C |
| ATOM | 1472 | CE1 | PHE | A 206 | 30.958 | 64.549 | 3.383 | 1.00 | 0.00 | C |
| ATOM | 1473 | CE2 | PHE | A 206 | 31.280 | 65.844 | 1.377 | 1.00 | 0.00 | C |
| ATOM | 1474 | CZ | PHE | A 206 | 31.514 | 65.652 | 2.738 | 1.00 | 0.00 | C |
| ATOM | 1475 | N | GLY | A 207 | 28.947 | 59.165 | −0.023 | 1.00 | 0.00 | N |
| ATOM | 1476 | CA | GLY | A 207 | 28.678 | 58.183 | −1.058 | 1.00 | 0.00 | C |
| ATOM | 1477 | C | GLY | A 207 | 29.978 | 57.408 | −1.190 | 1.00 | 0.00 | C |
| ATOM | 1478 | O | GLY | A 207 | 31.035 | 57.938 | −0.844 | 1.00 | 0.00 | O |
| ATOM | 1479 | N | HIS | A 208 | 29.920 | 56.173 | −1.687 | 1.00 | 0.00 | N |
| ATOM | 1480 | CA | HIS | A 208 | 31.122 | 55.351 | −1.806 | 1.00 | 0.00 | C |
| ATOM | 1481 | C | HIS | A 208 | 31.361 | 54.759 | −3.187 | 1.00 | 0.00 | C |
| ATOM | 1482 | O | HIS | A 208 | 30.419 | 54.418 | −3.909 | 1.00 | 0.00 | O |
| ATOM | 1483 | CB | HIS | A 208 | 31.070 | 54.238 | −0.760 | 1.00 | 0.00 | C |
| ATOM | 1484 | CG | HIS | A 208 | 31.161 | 54.739 | 0.648 | 1.00 | 0.00 | C |
| ATOM | 1485 | ND1 | HIS | A 208 | 32.357 | 55.086 | 1.236 | 1.00 | 0.00 | N |
| ATOM | 1486 | CD2 | HIS | A 208 | 30.203 | 54.976 | 1.575 | 1.00 | 0.00 | C |
| ATOM | 1487 | CE1 | HIS | A 208 | 32.133 | 55.513 | 2.466 | 1.00 | 0.00 | C |
| ATOM | 1488 | NE2 | HIS | A 208 | 30.834 | 55.457 | 2.696 | 1.00 | 0.00 | N |
| ATOM | 1489 | N | SER | A 209 | 32.635 | 54.625 | −3.537 | 1.00 | 0.00 | N |
| ATOM | 1490 | CA | SER | A 209 | 33.036 | 54.106 | −4.841 | 1.00 | 0.00 | C |
| ATOM | 1491 | C | SER | A 209 | 33.793 | 52.783 | −4.782 | 1.00 | 0.00 | C |
| ATOM | 1492 | O | SER | A 209 | 34.595 | 52.560 | −3.872 | 1.00 | 0.00 | O |
| ATOM | 1493 | CB | SER | A 209 | 33.916 | 55.138 | −5.551 | 1.00 | 0.00 | C |
| ATOM | 1494 | OG | SER | A 209 | 34.484 | 54.592 | −6.734 | 1.00 | 0.00 | O |
| ATOM | 1495 | N | PRO | A 210 | 33.552 | 51.893 | −5.764 | 1.00 | 0.00 | N |
| ATOM | 1496 | CA | PRO | A 210 | 34.224 | 50.593 | −5.819 | 1.00 | 0.00 | C |
| ATOM | 1497 | C | PRO | A 210 | 35.704 | 50.764 | −6.148 | 1.00 | 0.00 | C |
| ATOM | 1498 | O | PRO | A 210 | 36.479 | 49.812 | −6.074 | 1.00 | 0.00 | O |
| ATOM | 1499 | CB | PRO | A 210 | 33.451 | 49.847 | −6.907 | 1.00 | 0.00 | C |
| ATOM | 1500 | CG | PRO | A 210 | 33.031 | 50.950 | −7.830 | 1.00 | 0.00 | C |
| ATOM | 1501 | CD | PRO | A 210 | 32.565 | 52.014 | −6.853 | 1.00 | 0.00 | C |
| ATOM | 1502 | N | THR | A 211 | 36.103 | 51.978 | −6.518 | 1.00 | 0.00 | N |
| ATOM | 1503 | CA | THR | A 211 | 37.513 | 52.217 | −6.789 | 1.00 | 0.00 | C |
| ATOM | 1504 | C | THR | A 211 | 38.309 | 51.961 | −5.503 | 1.00 | 0.00 | C |
| ATOM | 1505 | O | THR | A 211 | 39.481 | 51.583 | −5.553 | 1.00 | 0.00 | O |
| ATOM | 1506 | CB | THR | A 211 | 37.768 | 53.668 | −7.260 | 1.00 | 0.00 | C |
| ATOM | 1507 | OG1 | THR | A 211 | 37.238 | 53.835 | −8.580 | 1.00 | 0.00 | O |
| ATOM | 1508 | CG2 | THR | A 211 | 39.265 | 53.974 | −7.280 | 1.00 | 0.00 | C |
| ATOM | 1509 | N | MET | A 212 | 37.671 | 52.153 | −4.351 | 1.00 | 0.00 | N |
| ATOM | 1510 | CA | MET | A 212 | 38.360 | 51.938 | −3.078 | 1.00 | 0.00 | C |
| ATOM | 1511 | C | MET | A 212 | 38.742 | 50.467 | −2.866 | 1.00 | 0.00 | C |
| ATOM | 1512 | O | MET | A 212 | 39.913 | 50.154 | −2.630 | 1.00 | 0.00 | O |
| ATOM | 1513 | CB | MET | A 212 | 37.514 | 52.452 | −1.909 | 1.00 | 0.00 | C |
| ATOM | 1514 | CG | MET | A 212 | 37.203 | 53.944 | −1.967 | 1.00 | 0.00 | C |
| ATOM | 1515 | SD | MET | A 212 | 38.661 | 55.001 | −2.244 | 1.00 | 0.00 | S |
| ATOM | 1516 | CE | MET | A 212 | 39.452 | 54.939 | −0.628 | 1.00 | 0.00 | C |
| ATOM | 1517 | N | PRO | A 213 | 37.769 | 49.541 | −2.930 | 1.00 | 0.00 | N |
| ATOM | 1518 | CA | PRO | A 213 | 38.188 | 48.148 | −2.733 | 1.00 | 0.00 | C |
| ATOM | 1519 | C | PRO | A 213 | 39.170 | 47.704 | −3.822 | 1.00 | 0.00 | C |
| ATOM | 1520 | O | PRO | A 213 | 40.036 | 46.862 | −3.583 | 1.00 | 0.00 | O |
| ATOM | 1521 | CB | PRO | A 213 | 36.862 | 47.371 | −2.754 | 1.00 | 0.00 | C |
| ATOM | 1522 | CG | PRO | A 213 | 35.922 | 48.287 | −3.530 | 1.00 | 0.00 | C |
| ATOM | 1523 | CD | PRO | A 213 | 36.302 | 49.649 | −3.011 | 1.00 | 0.00 | C |
| ATOM | 1524 | N | TYR | A 214 | 39.038 | 48.274 | −5.018 | 1.00 | 0.00 | N |
| ATOM | 1525 | CA | TYR | A 214 | 39.944 | 47.943 | −6.117 | 1.00 | 0.00 | C |
| ATOM | 1526 | C | TYR | A 214 | 41.390 | 48.211 | −5.698 | 1.00 | 0.00 | C |
| ATOM | 1527 | O | TYR | A 214 | 42.257 | 47.337 | −5.793 | 1.00 | 0.00 | O |
| ATOM | 1528 | CB | TYR | A 214 | 39.628 | 48.788 | −7.346 | 1.00 | 0.00 | C |
| ATOM | 1529 | CG | TYR | A 214 | 40.553 | 48.531 | −8.517 | 1.00 | 0.00 | C |
| ATOM | 1530 | CD1 | TYR | A 214 | 40.385 | 47.413 | −9.335 | 1.00 | 0.00 | C |
| ATOM | 1531 | CD2 | TYR | A 214 | 41.579 | 49.422 | −8.823 | 1.00 | 0.00 | C |
| ATOM | 1532 | CE1 | TYR | A 214 | 41.215 | 47.197 | −10.439 | 1.00 | 0.00 | C |
| ATOM | 1533 | CE2 | TYR | A 214 | 42.412 | 49.215 | −9.918 | 1.00 | 0.00 | C |
| ATOM | 1534 | CZ | TYR | A 214 | 42.224 | 48.107 | −10.725 | 1.00 | 0.00 | C |
| ATOM | 1535 | OH | TYR | A 214 | 43.026 | 47.934 | −11.834 | 1.00 | 0.00 | O |
| ATOM | 1536 | N | ILE | A 215 | 41.640 | 49.435 | −5.241 | 1.00 | 0.00 | N |
| ATOM | 1537 | CA | ILE | A 215 | 42.969 | 49.848 | −4.804 | 1.00 | 0.00 | C |
| ATOM | 1538 | C | ILE | A 215 | 43.399 | 49.135 | −3.516 | 1.00 | 0.00 | C |
| ATOM | 1539 | O | ILE | A 215 | 44.529 | 48.660 | −3.403 | 1.00 | 0.00 | O |
| ATOM | 1540 | CB | ILE | A 215 | 43.008 | 51.384 | −4.567 | 1.00 | 0.00 | C |
| ATOM | 1541 | CG1 | ILE | A 215 | 42.745 | 52.118 | −5.885 | 1.00 | 0.00 | C |
| ATOM | 1542 | CG2 | ILE | A 215 | 44.352 | 51.797 | −3.972 | 1.00 | 0.00 | C |
| ATOM | 1543 | CD1 | ILE | A 215 | 42.721 | 53.637 | −5.749 | 1.00 | 0.00 | C |
| ATOM | 1544 | N | LEU | A 216 | 42.494 | 49.062 | −2.548 | 1.00 | 0.00 | N |
| ATOM | 1545 | CA | LEU | A 216 | 42.803 | 48.419 | −1.273 | 1.00 | 0.00 | C |
| ATOM | 1546 | C | LEU | A 216 | 43.141 | 46.932 | −1.412 | 1.00 | 0.00 | C |
| ATOM | 1547 | O | LEU | A 216 | 44.108 | 46.452 | −0.817 | 1.00 | 0.00 | O |
| ATOM | 1548 | CB | LEU | A 216 | 41.630 | 48.594 | −0.300 | 1.00 | 0.00 | C |
| ATOM | 1549 | CG | LEU | A 216 | 41.247 | 50.039 | 0.061 | 1.00 | 0.00 | C |
| ATOM | 1550 | CD1 | LEU | A 216 | 39.929 | 50.049 | 0.827 | 1.00 | 0.00 | C |
| ATOM | 1551 | CD2 | LEU | A 216 | 42.365 | 50.678 | 0.891 | 1.00 | 0.00 | C |
| ATOM | 1552 | N | GLN | A 217 | 42.347 | 46.207 | −2.197 | 1.00 | 0.00 | N |
| ATOM | 1553 | CA | GLN | A 217 | 42.568 | 44.776 | −2.388 | 1.00 | 0.00 | C |
| ATOM | 1554 | C | GLN | A 217 | 43.925 | 44.525 | −3.051 | 1.00 | 0.00 | C |
| ATOM | 1555 | O | GLN | A 217 | 44.556 | 43.490 | −2.826 | 1.00 | 0.00 | O |
| ATOM | 1556 | CB | GLN | A 217 | 41.418 | 44.191 | −3.218 | 1.00 | 0.00 | C |
| ATOM | 1557 | CG | GLN | A 217 | 41.365 | 42.669 | −3.307 | 1.00 | 0.00 | C |
| ATOM | 1558 | CD | GLN | A 217 | 42.293 | 42.120 | −4.364 | 1.00 | 0.00 | C |
| ATOM | 1559 | OE1 | GLN | A 217 | 42.498 | 42.747 | −5.401 | 1.00 | 0.00 | O |
| ATOM | 1560 | NE2 | GLN | A 217 | 42.849 | 40.938 | −4.115 | 1.00 | 0.00 | N |
| ATOM | 1561 | N | LYS | A 218 | 44.376 | 45.485 | −3.856 | 1.00 | 0.00 | N |
| ATOM | 1562 | CA | LYS | A 218 | 45.666 | 45.386 | −4.537 | 1.00 | 0.00 | C |
| ATOM | 1563 | C | LYS | A 218 | 46.763 | 46.022 | −3.681 | 1.00 | 0.00 | C |
| ATOM | 1564 | O | LYS | A 218 | 47.906 | 46.170 | −4.114 | 1.00 | 0.00 | O |
| ATOM | 1565 | CB | LYS | A 218 | 45.598 | 46.082 | −5.904 | 1.00 | 0.00 | C |
| ATOM | 1566 | CG | LYS | A 218 | 44.800 | 45.307 | −6.956 | 1.00 | 0.00 | C |
| ATOM | 1567 | CD | LYS | A 218 | 44.608 | 46.122 | −8.238 | 1.00 | 0.00 | C |
| ATOM | 1568 | CE | LYS | A 218 | 44.225 | 45.238 | −9.419 | 1.00 | 0.00 | C |
| ATOM | 1569 | NZ | LYS | A 218 | 43.094 | 44.308 | −9.142 | 1.00 | 0.00 | N |
| ATOM | 1570 | N | SER | A 219 | 46.401 | 46.396 | −2.460 | 1.00 | 0.00 | N |
| ATOM | 1571 | CA | SER | A 219 | 47.343 | 47.014 | −1.541 | 1.00 | 0.00 | C |
| ATOM | 1572 | C | SER | A 219 | 47.419 | 46.231 | −0.227 | 1.00 | 0.00 | C |
| ATOM | 1573 | O | SER | A 219 | 47.749 | 46.782 | 0.827 | 1.00 | 0.00 | O |
| ATOM | 1574 | CB | SER | A 219 | 46.934 | 48.469 | −1.288 | 1.00 | 0.00 | C |
| ATOM | 1575 | OG | SER | A 219 | 47.797 | 49.081 | −0.345 | 1.00 | 0.00 | O |
| ATOM | 1576 | N | GLY | A 220 | 47.102 | 44.940 | −0.305 | 1.00 | 0.00 | N |
| ATOM | 1577 | CA | GLY | A 220 | 47.173 | 44.079 | 0.865 | 1.00 | 0.00 | C |
| ATOM | 1578 | C | GLY | A 220 | 45.984 | 44.045 | 1.810 | 1.00 | 0.00 | C |
| ATOM | 1579 | O | GLY | A 220 | 45.997 | 43.291 | 2.784 | 1.00 | 0.00 | O |
| ATOM | 1580 | N | PHE | A 221 | 44.948 | 44.831 | 1.539 | 1.00 | 0.00 | N |
| ATOM | 1581 | CA | PHE | A 221 | 43.795 | 44.848 | 2.433 | 1.00 | 0.00 | C |
| ATOM | 1582 | C | PHE | A 221 | 42.940 | 43.596 | 2.363 | 1.00 | 0.00 | C |
| ATOM | 1583 | O | PHE | A 221 | 42.891 | 42.918 | 1.340 | 1.00 | 0.00 | O |
| ATOM | 1584 | CB | PHE | A 221 | 42.911 | 46.070 | 2.164 | 1.00 | 0.00 | C |
| ATOM | 1585 | CG | PHE | A 221 | 43.469 | 47.350 | 2.709 | 1.00 | 0.00 | C |
| ATOM | 1586 | CD1 | PHE | A 221 | 44.587 | 47.941 | 2.127 | 1.00 | 0.00 | C |
| ATOM | 1587 | CD2 | PHE | A 221 | 42.883 | 47.961 | 3.813 | 1.00 | 0.00 | C |
| ATOM | 1588 | CE1 | PHE | A 221 | 45.115 | 49.126 | 2.637 | 1.00 | 0.00 | C |
| ATOM | 1589 | CE2 | PHE | A 221 | 43.404 | 49.150 | 4.334 | 1.00 | 0.00 | C |
| ATOM | 1590 | CZ | PHE | A 221 | 44.522 | 49.732 | 3.743 | 1.00 | 0.00 | C |
| ATOM | 1591 | N | LYS | A 222 | 42.262 | 43.305 | 3.468 | 1.00 | 0.00 | N |
| ATOM | 1592 | CA | LYS | A 222 | 41.390 | 42.147 | 3.550 | 1.00 | 0.00 | C |
| ATOM | 1593 | C | LYS | A 222 | 39.978 | 42.530 | 3.970 | 1.00 | 0.00 | C |
| ATOM | 1594 | O | LYS | A 222 | 39.048 | 41.741 | 3.810 | 1.00 | 0.00 | O |
| ATOM | 1595 | CB | LYS | A 222 | 41.974 | 41.124 | 4.525 | 1.00 | 0.00 | C |
| ATOM | 1596 | CG | LYS | A 222 | 43.170 | 40.390 | 3.943 | 1.00 | 0.00 | C |
| ATOM | 1597 | CD | LYS | A 222 | 43.812 | 39.451 | 4.940 | 1.00 | 0.00 | C |
| ATOM | 1598 | CE | LYS | A 222 | 44.810 | 38.535 | 4.239 | 1.00 | 0.00 | C |
| ATOM | 1599 | NZ | LYS | A 222 | 45.750 | 39.298 | 3.363 | 1.00 | 0.00 | N |
| ATOM | 1600 | N | ASN | A 223 | 39.819 | 43.746 | 4.493 | 1.00 | 0.00 | N |
| ATOM | 1601 | CA | ASN | A 223 | 38.511 | 44.222 | 4.940 | 1.00 | 0.00 | C |
| ATOM | 1602 | C | ASN | A 223 | 38.347 | 45.738 | 4.828 | 1.00 | 0.00 | C |
| ATOM | 1603 | O | ASN | A 223 | 39.322 | 46.483 | 4.892 | 1.00 | 0.00 | O |
| ATOM | 1604 | CB | ASN | A 223 | 38.270 | 43.832 | 6.404 | 1.00 | 0.00 | C |
| ATOM | 1605 | CG | ASN | A 223 | 38.360 | 42.340 | 6.640 | 1.00 | 0.00 | C |
| ATOM | 1606 | OD1 | ASN | A 223 | 39.390 | 41.826 | 7.096 | 1.00 | 0.00 | O |
| ATOM | 1607 | ND2 | ASN | A 223 | 37.284 | 41.631 | 6.330 | 1.00 | 0.00 | N |
| ATOM | 1608 | N | MET | A 224 | 37.104 | 46.186 | 4.670 | 1.00 | 0.00 | N |
| ATOM | 1609 | CA | MET | A 224 | 36.807 | 47.614 | 4.599 | 1.00 | 0.00 | C |
| ATOM | 1610 | C | MET | A 224 | 35.408 | 47.896 | 5.141 | 1.00 | 0.00 | C |
| ATOM | 1611 | O | MET | A 224 | 34.522 | 47.035 | 5.108 | 1.00 | 0.00 | O |
| ATOM | 1612 | CB | MET | A 224 | 36.922 | 48.137 | 3.166 | 1.00 | 0.00 | C |
| ATOM | 1613 | CG | MET | A 224 | 35.850 | 47.637 | 2.214 | 1.00 | 0.00 | C |
| ATOM | 1614 | SD | MET | A 224 | 36.055 | 48.407 | 0.602 | 1.00 | 0.00 | S |
| ATOM | 1615 | CE | MET | A 224 | 35.417 | 50.048 | 0.921 | 1.00 | 0.00 | C |
| ATOM | 1616 | N | LEU | A 225 | 35.226 | 49.110 | 5.649 | 1.00 | 0.00 | N |
| ATOM | 1617 | CA | LEU | A 225 | 33.955 | 49.534 | 6.219 | 1.00 | 0.00 | C |
| ATOM | 1618 | C | LEU | A 225 | 33.527 | 50.873 | 5.628 | 1.00 | 0.00 | C |
| ATOM | 1619 | O | LEU | A 225 | 34.351 | 51.769 | 5.456 | 1.00 | 0.00 | O |
| ATOM | 1620 | CB | LEU | A 225 | 34.096 | 49.651 | 7.739 | 1.00 | 0.00 | C |
| ATOM | 1621 | CG | LEU | A 225 | 32.938 | 50.271 | 8.531 | 1.00 | 0.00 | C |
| ATOM | 1622 | CD1 | LEU | A 225 | 32.940 | 49.699 | 9.937 | 1.00 | 0.00 | C |
| ATOM | 1623 | CD2 | LEU | A 225 | 33.064 | 51.808 | 8.553 | 1.00 | 0.00 | C |
| ATOM | 1624 | N | ILE | A 226 | 32.239 | 50.999 | 5.317 | 1.00 | 0.00 | N |
| ATOM | 1625 | CA | ILE | A 226 | 31.697 | 52.231 | 4.749 | 1.00 | 0.00 | C |
| ATOM | 1626 | C | ILE | A 226 | 30.439 | 52.632 | 5.518 | 1.00 | 0.00 | C |
| ATOM | 1627 | O | ILE | A 226 | 29.857 | 51.808 | 6.228 | 1.00 | 0.00 | O |
| ATOM | 1628 | CB | ILE | A 226 | 31.368 | 52.058 | 3.244 | 1.00 | 0.00 | C |
| ATOM | 1629 | CG1 | ILE | A 226 | 30.308 | 50.972 | 3.049 | 1.00 | 0.00 | C |
| ATOM | 1630 | CG2 | ILE | A 226 | 32.642 | 51.695 | 2.479 | 1.00 | 0.00 | C |
| ATOM | 1631 | CD1 | ILE | A 226 | 29.889 | 50.776 | 1.595 | 1.00 | 0.00 | C |
| ATOM | 1632 | N | GLN | A 227 | 30.014 | 53.886 | 5.377 | 1.00 | 0.00 | N |
| ATOM | 1633 | CA | GLN | A 227 | 28.848 | 54.367 | 6.115 | 1.00 | 0.00 | C |
| ATOM | 1634 | C | GLN | A 227 | 27.809 | 55.189 | 5.351 | 1.00 | 0.00 | C |
| ATOM | 1635 | O | GLN | A 227 | 26.613 | 54.893 | 5.409 | 1.00 | 0.00 | O |
| ATOM | 1636 | CB | GLN | A 227 | 29.332 | 55.171 | 7.330 | 1.00 | 0.00 | C |
| ATOM | 1637 | CG | GLN | A 227 | 28.377 | 56.258 | 7.841 | 1.00 | 0.00 | C |
| ATOM | 1638 | CD | GLN | A 227 | 27.056 | 55.721 | 8.361 | 1.00 | 0.00 | C |
| ATOM | 1639 | OE1 | GLN | A 227 | 26.954 | 54.563 | 8.765 | 1.00 | 0.00 | O |
| ATOM | 1640 | NE2 | GLN | A 227 | 26.037 | 56.577 | 8.376 | 1.00 | 0.00 | N |
| ATOM | 1641 | N | ARG | A 228 | 28.249 | 56.227 | 4.646 | 1.00 | 0.00 | N |
| ATOM | 1642 | CA | ARG | A 228 | 27.298 | 57.072 | 3.944 | 1.00 | 0.00 | C |
| ATOM | 1643 | C | ARG | A 228 | 26.756 | 56.523 | 2.632 | 1.00 | 0.00 | C |
| ATOM | 1644 | O | ARG | A 228 | 27.318 | 56.746 | 1.560 | 1.00 | 0.00 | O |
| ATOM | 1645 | CB | ARG | A 228 | 27.887 | 58.472 | 3.731 | 1.00 | 0.00 | C |
| ATOM | 1646 | CG | ARG | A 228 | 28.057 | 59.260 | 5.029 | 1.00 | 0.00 | C |
| ATOM | 1647 | CD | ARG | A 228 | 28.403 | 60.729 | 4.778 | 1.00 | 0.00 | C |
| ATOM | 1648 | NE | ARG | A 228 | 28.639 | 61.461 | 6.027 | 1.00 | 0.00 | N |
| ATOM | 1649 | CZ | ARG | A 228 | 27.683 | 61.924 | 6.831 | 1.00 | 0.00 | C |
| ATOM | 1650 | NH1 | ARG | A 228 | 26.401 | 61.743 | 6.526 | 1.00 | 0.00 | N |
| ATOM | 1651 | NH2 | ARG | A 228 | 28.007 | 62.560 | 7.953 | 1.00 | 0.00 | N |
| ATOM | 1652 | N | THR | A 229 | 25.652 | 55.791 | 2.739 | 1.00 | 0.00 | N |
| ATOM | 1653 | CA | THR | A 229 | 24.977 | 55.233 | 1.579 | 1.00 | 0.00 | C |
| ATOM | 1654 | C | THR | A 229 | 23.509 | 55.617 | 1.731 | 1.00 | 0.00 | C |
| ATOM | 1655 | O | THR | A 229 | 23.038 | 55.854 | 2.849 | 1.00 | 0.00 | O |
| ATOM | 1656 | CB | THR | A 229 | 25.130 | 53.688 | 1.504 | 1.00 | 0.00 | C |
| ATOM | 1657 | OG1 | THR | A 229 | 24.551 | 53.078 | 2.665 | 1.00 | 0.00 | O |
| ATOM | 1658 | CG1 | THR | A 229 | 26.609 | 53.310 | 1.419 | 1.00 | 0.00 | C |
| ATOM | 1659 | N | HIS | A 230 | 22.798 | 55.694 | 0.611 | 1.00 | 0.00 | N |
| ATOM | 1660 | CA | HIS | A 230 | 21.383 | 56.077 | 0.594 | 1.00 | 0.00 | C |
| ATOM | 1661 | C | HIS | A 230 | 20.573 | 55.338 | 1.661 | 1.00 | 0.00 | C |
| ATOM | 1662 | O | HIS | A 230 | 20.679 | 54.120 | 1.797 | 1.00 | 0.00 | O |
| ATOM | 1663 | CB | HIS | A 230 | 20.804 | 55.798 | −0.799 | 1.00 | 0.00 | C |
| ATOM | 1664 | CG | HIS | A 230 | 19.543 | 56.549 | −1.099 | 1.00 | 0.00 | C |
| ATOM | 1665 | ND1 | HIS | A 230 | 18.378 | 56.370 | −0.385 | 1.00 | 0.00 | N |
| ATOM | 1666 | CD2 | HIS | A 230 | 19.265 | 57.476 | −2.047 | 1.00 | 0.00 | C |
| ATOM | 1667 | CE1 | HIS | A 230 | 17.436 | 57.155 | −0.879 | 1.00 | 0.00 | C |
| ATOM | 1668 | NE2 | HIS | A 230 | 17.948 | 57.837 | −1.888 | 1.00 | 0.00 | N |
| ATOM | 1669 | N | TYR | A 231 | 19.756 | 56.074 | 2.415 | 1.00 | 0.00 | N |
| ATOM | 1670 | CA | TYR | A 231 | 18.958 | 55.448 | 3.466 | 1.00 | 0.00 | C |
| ATOM | 1671 | C | TYR | A 231 | 18.081 | 54.310 | 2.940 | 1.00 | 0.00 | C |
| ATOM | 1672 | O | TYR | A 231 | 17.788 | 53.361 | 3.666 | 1.00 | 0.00 | O |
| ATOM | 1673 | CB | TYR | A 231 | 18.105 | 56.497 | 4.200 | 1.00 | 0.00 | C |
| ATOM | 1674 | CG | TYR | A 231 | 17.122 | 57.265 | 3.336 | 1.00 | 0.00 | C |
| ATOM | 1675 | CD1 | TYR | A 231 | 15.842 | 56.767 | 3.085 | 1.00 | 0.00 | C |
| ATOM | 1676 | CD2 | TYR | A 231 | 17.473 | 58.497 | 2.777 | 1.00 | 0.00 | C |
| ATOM | 1677 | CE1 | TYR | A 231 | 14.935 | 57.477 | 2.301 | 1.00 | 0.00 | C |
| ATOM | 1678 | CE2 | TYR | A 231 | 16.574 | 59.214 | 1.992 | 1.00 | 0.00 | C |
| ATOM | 1679 | CZ | TYR | A 231 | 15.308 | 58.698 | 1.758 | 1.00 | 0.00 | C |
| ATOM | 1680 | OH | TYR | A 231 | 14.420 | 59.401 | 0.980 | 1.00 | 0.00 | O |
| ATOM | 1681 | N | SER | A 232 | 17.673 | 54.395 | 1.679 | 1.00 | 0.00 | N |
| ATOM | 1682 | CA | SER | A 232 | 16.846 | 53.345 | 1.089 | 1.00 | 0.00 | C |
| ATOM | 1683 | C | SER | A 232 | 17.649 | 52.069 | 0.857 | 1.00 | 0.00 | C |
| ATOM | 1684 | O | SER | A 232 | 17.119 | 50.964 | 0.970 | 1.00 | 0.00 | O |
| ATOM | 1685 | CB | SER | A 232 | 16.238 | 53.821 | −0.233 | 1.00 | 0.00 | C |
| ATOM | 1686 | OG | SER | A 232 | 15.249 | 54.809 | −0.007 | 1.00 | 0.00 | O |
| ATOM | 1687 | N | VAL | A 233 | 18.930 | 52.227 | 0.533 | 1.00 | 0.00 | N |
| ATOM | 1688 | CA | VAL | A 233 | 19.807 | 51.085 | 0.298 | 1.00 | 0.00 | C |
| ATOM | 1689 | C | VAL | A 233 | 20.094 | 50.365 | 1.618 | 1.00 | 0.00 | C |
| ATOM | 1690 | O | VAL | A 233 | 20.097 | 49.132 | 1.680 | 1.00 | 0.00 | O |
| ATOM | 1691 | CB | VAL | A 233 | 21.134 | 51.537 | −0.354 | 1.00 | 0.00 | C |
| ATOM | 1692 | CG1 | VAL | A 233 | 22.119 | 50.375 | −0.405 | 1.00 | 0.00 | C |
| ATOM | 1693 | CG2 | VAL | A 233 | 20.860 | 52.054 | −1.763 | 1.00 | 0.00 | C |
| ATOM | 1694 | N | LYS | A 234 | 20.327 | 51.140 | 2.670 | 1.00 | 0.00 | N |
| ATOM | 1695 | CA | LYS | A 234 | 20.582 | 50.573 | 3.988 | 1.00 | 0.00 | C |
| ATOM | 1696 | C | LYS | A 234 | 19.394 | 49.707 | 4.407 | 1.00 | 0.00 | C |
| ATOM | 1697 | O | LYS | A 234 | 19.567 | 48.587 | 4.884 | 1.00 | 0.00 | O |
| ATOM | 1698 | CB | LYS | A 234 | 20.799 | 51.695 | 5.013 | 1.00 | 0.00 | C |
| ATOM | 1699 | CG | LYS | A 234 | 22.143 | 52.414 | 4.889 | 1.00 | 0.00 | C |
| ATOM | 1700 | CD | LYS | A 234 | 22.200 | 53.646 | 5.791 | 1.00 | 0.00 | C |
| ATOM | 1701 | CE | LYS | A 234 | 23.575 | 54.324 | 5.751 | 1.00 | 0.00 | C |
| ATOM | 1702 | NZ | LYS | A 234 | 24.576 | 53.708 | 6.681 | 1.00 | 0.00 | N |
| ATOM | 1703 | NZ | LYS | A 235 | 18.186 | 50.229 | 4.213 | 1.00 | 0.00 | N |
| ATOM | 1704 | CA | LYS | A 235 | 16.974 | 49.497 | 4.578 | 1.00 | 0.00 | C |
| ATOM | 1705 | C | LYS | A 235 | 16.845 | 48.206 | 3.777 | 1.00 | 0.00 | C |
| ATOM | 1706 | O | LYS | A 235 | 16.595 | 47.138 | 4.337 | 1.00 | 0.00 | O |
| ATOM | 1707 | CB | LYS | A 235 | 15.739 | 50.373 | 4.351 | 1.00 | 0.00 | C |
| ATOM | 1708 | CG | LYS | A 235 | 14.445 | 49.774 | 4.890 | 1.00 | 0.00 | C |
| ATOM | 1709 | CD | LYS | A 235 | 13.270 | 50.689 | 4.608 | 1.00 | 0.00 | C |
| ATOM | 1710 | CE | LYS | A 235 | 11.977 | 50.108 | 5.154 | 1.00 | 0.00 | C |
| ATOM | 1711 | NZ | LYS | A 235 | 10.807 | 50.957 | 4.792 | 1.00 | 0.00 | N |
| ATOM | 1712 | N | GLU | A 236 | 17.025 | 48.307 | 2.464 | 1.00 | 0.00 | N |
| ATOM | 1713 | CA | GLU | A 236 | 16.926 | 47.149 | 1.580 | 1.00 | 0.00 | C |
| ATOM | 1714 | C | GLU | A 236 | 17.941 | 46.058 | 1.928 | 1.00 | 0.00 | C |
| ATOM | 1715 | O | GLU | A 236 | 17.584 | 44.888 | 2.073 | 1.00 | 0.00 | O |
| ATOM | 1716 | CB | GLU | A 236 | 17.121 | 47.587 | 0.125 | 1.00 | 0.00 | C |
| ATOM | 1717 | CG | GLU | A 236 | 16.922 | 46.474 | −0.897 | 1.00 | 0.00 | C |
| ATOM | 1718 | CD | GLU | A 236 | 15.468 | 46.035 | −1.029 | 1.00 | 0.00 | C |
| ATOM | 1719 | OE1 | GLU | A 236 | 15.200 | 45.126 | −1.840 | 1.00 | 0.00 | O |
| ATOM | 1720 | OE2 | GLU | A 236 | 14.594 | 46.596 | −0.332 | 1.00 | 0.00 | O |
| ATOM | 1721 | N | LEU | A 237 | 19.210 | 46.432 | 2.063 | 1.00 | 0.00 | N |
| ATOM | 1722 | CA | LEU | A 237 | 20.236 | 45.450 | 2.387 | 1.00 | 0.00 | C |
| ATOM | 1723 | C | LEU | A 237 | 20.065 | 44.897 | 3.800 | 1.00 | 0.00 | C |
| ATOM | 1724 | O | LEU | A 237 | 20.318 | 43.717 | 4.043 | 1.00 | 0.00 | O |
| ATOM | 1725 | CB | LEU | A 237 | 21.632 | 46.056 | 2.220 | 1.00 | 0.00 | C |
| ATOM | 1726 | CG | LEU | A 237 | 21.974 | 46.530 | 0.802 | 1.00 | 0.00 | C |
| ATOM | 1727 | CD1 | LEU | A 237 | 23.357 | 47.152 | 0.803 | 1.00 | 0.00 | C |
| ATOM | 1728 | CD2 | LEU | A 237 | 21.912 | 45.359 | −0.178 | 1.00 | 0.00 | C |
| ATOM | 1729 | N | ALA | A 238 | 19.630 | 45.742 | 4.729 | 1.00 | 0.00 | N |
| ATOM | 1730 | CA | ALA | A 238 | 19.428 | 45.297 | 6.102 | 1.00 | 0.00 | C |
| ATOM | 1731 | C | ALA | A 238 | 18.379 | 44.184 | 6.150 | 1.00 | 0.00 | C |
| ATOM | 1732 | O | ALA | A 238 | 18.564 | 43.172 | 6.833 | 1.00 | 0.00 | O |
| ATOM | 1733 | CB | ALA | A 238 | 18.988 | 46.465 | 6.976 | 1.00 | 0.00 | C |
| ATOM | 1734 | N | GLN | A 239 | 17.286 | 44.374 | 5.420 | 1.00 | 0.00 | N |
| ATOM | 1735 | CA | GLN | A 239 | 16.203 | 43.394 | 5.393 | 1.00 | 0.00 | C |
| ATOM | 1736 | C | GLN | A 239 | 16.652 | 42.029 | 4.871 | 1.00 | 0.00 | C |
| ATOM | 1737 | O | GLN | A 239 | 16.079 | 41.002 | 5.229 | 1.00 | 0.00 | O |
| ATOM | 1738 | CB | GLN | A 239 | 15.037 | 43.922 | 4.548 | 1.00 | 0.00 | C |
| ATOM | 1739 | CG | GLN | A 239 | 14.452 | 45.234 | 5.069 | 1.00 | 0.00 | C |
| ATOM | 1740 | CD | GLN | A 239 | 13.274 | 45.734 | 4.248 | 1.00 | 0.00 | C |
| ATOM | 1741 | OE1 | GLN | A 239 | 13.349 | 45.823 | 3.021 | 1.00 | 0.00 | O |
| ATOM | 1742 | NE2 | GLN | A 239 | 12.181 | 46.072 | 4.926 | 1.00 | 0.00 | N |
| ATOM | 1743 | N | GLN | A 240 | 17.679 | 42.017 | 4.028 | 1.00 | 0.00 | N |
| ATOM | 1744 | CA | GLN | A 240 | 18.187 | 40.766 | 3.472 | 1.00 | 0.00 | C |
| ATOM | 1745 | C | GLN | A 240 | 19.494 | 40.349 | 4.138 | 1.00 | 0.00 | C |
| ATOM | 1746 | O | GLN | A 240 | 20.113 | 39.364 | 3.735 | 1.00 | 0.00 | O |
| ATOM | 1747 | CB | GLN | A 240 | 18.416 | 40.921 | 1.968 | 1.00 | 0.00 | C |
| ATOM | 1748 | CG | GLN | A 240 | 17.193 | 41.409 | 1.212 | 1.00 | 0.00 | C |
| ATOM | 1749 | CD | GLN | A 240 | 16.058 | 40.409 | 1.242 | 1.00 | 0.00 | C |
| ATOM | 1750 | OE1 | GLN | A 240 | 14.902 | 40.762 | 1.011 | 1.00 | 0.00 | O |
| ATOM | 1751 | NE2 | GLN | A 240 | 16.382 | 39.149 | 1.514 | 1.00 | 0.00 | N |
| ATOM | 1752 | N | ARG | A 241 | 19.894 | 41.088 | 5.170 | 1.00 | 0.00 | N |
| ATOM | 1753 | CA | ARG | A 241 | 21.148 | 40.831 | 5.871 | 1.00 | 0.00 | C |
| ATOM | 1754 | C | ARG | A 241 | 22.282 | 40.786 | 4.852 | 1.00 | 0.00 | C |
| ATOM | 1755 | O | ARG | A 241 | 23.090 | 39.853 | 4.821 | 1.00 | 0.00 | O |
| ATOM | 1756 | CB | ARG | A 241 | 21.085 | 39.519 | 6.665 | 1.00 | 0.00 | C |
| ATOM | 1757 | CG | ARG | A 241 | 20.051 | 39.539 | 7.786 | 1.00 | 0.00 | C |
| ATOM | 1758 | CD | ARG | A 241 | 20.262 | 38.400 | 8.770 | 1.00 | 0.00 | C |
| ATOM | 1759 | NE | ARG | A 241 | 20.254 | 37.096 | 8.113 | 1.00 | 0.00 | N |
| ATOM | 1760 | CZ | ARG | A 241 | 20.626 | 35.960 | 8.699 | 1.00 | 0.00 | C |
| ATOM | 1761 | NH1 | ARG | A 241 | 21.038 | 35.964 | 9.962 | 1.00 | 0.00 | N |
| ATOM | 1762 | NH2 | ARG | A 241 | 20.595 | 34.821 | 8.019 | 1.00 | 0.00 | N |
| ATOM | 1763 | N | GLN | A 242 | 22.324 | 41.810 | 4.006 | 1.00 | 0.00 | N |
| ATOM | 1764 | CA | GLN | A 242 | 23.355 | 41.916 | 2.980 | 1.00 | 0.00 | C |
| ATOM | 1765 | C | GLN | A 242 | 24.195 | 43.177 | 3.199 | 1.00 | 0.00 | C |
| ATOM | 1766 | O | GLN | A 242 | 24.716 | 43.751 | 2.245 | 1.00 | 0.00 | O |
| ATOM | 1767 | CB | GLN | A 242 | 22.713 | 41.952 | 1.586 | 1.00 | 0.00 | C |
| ATOM | 1768 | CG | GLN | A 242 | 21.904 | 40.708 | 1.238 | 1.00 | 0.00 | C |
| ATOM | 1769 | CD | GLN | A 242 | 21.185 | 40.834 | −0.093 | 1.00 | 0.00 | C |
| ATOM | 1770 | OE1 | GLN | A 242 | 20.633 | 41.885 | −0.410 | 1.00 | 0.00 | O |
| ATOM | 1771 | NE2 | GLN | A 242 | 21.179 | 39.756 | −0.873 | 1.00 | 0.00 | N |
| ATOM | 1772 | N | LEU | A 243 | 24.320 | 43.596 | 4.460 | 1.00 | 0.00 | N |
| ATOM | 1773 | CA | LEU | A 243 | 25.106 | 44.779 | 4.810 | 1.00 | 0.00 | C |
| ATOM | 1774 | C | LEU | A 243 | 26.598 | 44.454 | 4.787 | 1.00 | 0.00 | C |
| ATOM | 1775 | O | LEU | A 243 | 27.441 | 45.351 | 4.712 | 1.00 | 0.00 | O |
| ATOM | 1776 | CB | LEU | A 243 | 24.691 | 45.310 | 6.186 | 1.00 | 0.00 | C |
| ATOM | 1777 | CG | LEU | A 243 | 23.302 | 45.958 | 6.224 | 1.00 | 0.00 | C |
| ATOM | 1778 | CD1 | LEU | A 243 | 22.897 | 46.244 | 7.667 | 1.00 | 0.00 | C |
| ATOM | 1779 | CD2 | LEU | A 243 | 23.319 | 47.251 | 5.397 | 1.00 | 0.00 | C |
| ATOM | 1780 | N | GLU | A 244 | 26.918 | 43.167 | 4.875 | 1.00 | 0.00 | N |
| ATOM | 1781 | CA | GLU | A 244 | 28.302 | 42.720 | 4.788 | 1.00 | 0.00 | C |
| ATOM | 1782 | C | GLU | A 244 | 28.326 | 41.894 | 3.521 | 1.00 | 0.00 | C |
| ATOM | 1783 | O | GLU | A 244 | 27.502 | 40.995 | 3.340 | 1.00 | 0.00 | O |
| ATOM | 1784 | CB | GLU | A 244 | 28.703 | 41.897 | 6.016 | 1.00 | 0.00 | C |
| ATOM | 1785 | CG | GLU | A 244 | 28.890 | 42.775 | 7.251 | 1.00 | 0.00 | C |
| ATOM | 1786 | CD | GLU | A 244 | 29.325 | 42.005 | 8.476 | 1.00 | 0.00 | C |
| ATOM | 1787 | OE1 | GLU | A 244 | 28.809 | 40.891 | 8.697 | 1.00 | 0.00 | O |
| ATOM | 1788 | OE2 | GLU | A 244 | 30.175 | 42.526 | 9.227 | 1.00 | 0.00 | O |
| ATOM | 1789 | N | PHE | A 245 | 29.259 | 42.214 | 2.631 | 1.00 | 0.00 | N |
| ATOM | 1790 | CA | PHE | A 245 | 29.333 | 41.533 | 1.349 | 1.00 | 0.00 | C |
| ATOM | 1791 | C | PHE | A 245 | 30.734 | 41.530 | 0.758 | 1.00 | 0.00 | C |
| ATOM | 1792 | O | PHE | A 245 | 31.611 | 42.272 | 1.198 | 1.00 | 0.00 | O |
| ATOM | 1793 | CB | PHE | A 245 | 28.368 | 42.225 | 0.377 | 1.00 | 0.00 | C |
| ATOM | 1794 | CG | PHE | A 245 | 28.491 | 43.730 | 0.370 | 1.00 | 0.00 | C |
| ATOM | 1795 | CD1 | PHE | A 245 | 29.473 | 44.364 | −0.388 | 1.00 | 0.00 | C |
| ATOM | 1796 | CD2 | PHE | A 245 | 27.643 | 44.511 | 1.155 | 1.00 | 0.00 | C |
| ATOM | 1797 | CE1 | PHE | A 245 | 29.610 | 45.767 | −0.363 | 1.00 | 0.00 | C |
| ATOM | 1798 | CE2 | PHE | A 245 | 27.770 | 45.907 | 1.190 | 1.00 | 0.00 | C |
| ATOM | 1799 | CZ | PHE | A 245 | 28.755 | 46.534 | 0.429 | 1.00 | 0.00 | C |
| ATOM | 1800 | N | LEU | A 246 | 30.936 | 40.676 | −0.238 | 1.00 | 0.00 | N |
| ATOM | 1801 | CA | LEU | A 246 | 32.211 | 40.590 | −0.931 | 1.00 | 0.00 | C |
| ATOM | 1802 | C | LEU | A 246 | 32.062 | 41.504 | −2.141 | 1.00 | 0.00 | C |
| ATOM | 1803 | O | LEU | A 246 | 31.450 | 41.138 | −3.144 | 1.00 | 0.00 | O |
| ATOM | 1804 | CB | LEU | A 246 | 32.481 | 39.143 | −1.351 | 1.00 | 0.00 | C |
| ATOM | 1805 | CG | LEU | A 246 | 32.796 | 38.222 | −0.162 | 1.00 | 0.00 | C |
| ATOM | 1806 | CD1 | LEU | A 246 | 32.737 | 36.755 | −0.577 | 1.00 | 0.00 | C |
| ATOM | 1807 | CD2 | LEU | A 246 | 34.176 | 38.572 | 0.374 | 1.00 | 0.00 | C |
| ATOM | 1808 | N | TRP | A 247 | 32.616 | 42.707 | −2.022 | 1.00 | 0.00 | N |
| ATOM | 1809 | CA | TRP | A 247 | 32.528 | 43.724 | −3.065 | 1.00 | 0.00 | C |
| ATOM | 1810 | C | TRP | A 247 | 33.547 | 43.514 | −4.182 | 1.00 | 0.00 | C |
| ATOM | 1811 | O | TRP | A 247 | 34.751 | 43.703 | −3.977 | 1.00 | 0.00 | O |
| ATOM | 1812 | CB | TRP | A 247 | 32.732 | 45.107 | −2.429 | 1.00 | 0.00 | C |
| ATOM | 1813 | CG | TRP | A 247 | 32.228 | 46.274 | −3.234 | 1.00 | 0.00 | C |
| ATOM | 1814 | CD1 | TRP | A 247 | 31.636 | 46.236 | −4.465 | 1.00 | 0.00 | C |
| ATOM | 1815 | CD2 | TRP | A 247 | 32.233 | 47.651 | −2.835 | 1.00 | 0.00 | C |
| ATOM | 1816 | NE1 | TRP | A 247 | 31.266 | 47.507 | −4.857 | 1.00 | 0.00 | N |
| ATOM | 1817 | CE2 | TRP | A 247 | 31.621 | 48.392 | −3.874 | 1.00 | 0.00 | C |
| ATOM | 1818 | CE3 | TRP | A 247 | 32.694 | 48.331 | −1.700 | 1.00 | 0.00 | C |
| ATOM | 1819 | CZ2 | TRP | A 247 | 31.458 | 49.782 | −3.809 | 1.00 | 0.00 | C |
| ATOM | 1820 | CZ3 | TRP | A 247 | 32.533 | 49.713 | −1.636 | 1.00 | 0.00 | C |
| ATOM | 1821 | CH2 | TRP | A 247 | 31.919 | 50.422 | −2.685 | 1.00 | 0.00 | C |
| ATOM | 1822 | N | ARG | A 248 | 33.062 | 43.119 | −5.358 | 1.00 | 0.00 | N |
| ATOM | 1823 | CA | ARG | A 248 | 33.929 | 42.908 | −6.511 | 1.00 | 0.00 | C |
| ATOM | 1824 | C | ARG | A 248 | 33.619 | 43.976 | −7.558 | 1.00 | 0.00 | C |
| ATOM | 1825 | O | ARG | A 248 | 32.567 | 44.618 | −7.509 | 1.00 | 0.00 | O |
| ATOM | 1826 | CB | ARG | A 248 | 33.700 | 41.521 | −7.126 | 1.00 | 0.00 | C |
| ATOM | 1827 | CG | ARG | A 248 | 32.350 | 41.366 | −7.822 | 1.00 | 0.00 | C |
| ATOM | 1828 | CD | ARG | A 248 | 32.291 | 40.067 | −8.632 | 1.00 | 0.00 | C |
| ATOM | 1829 | NE | ARG | A 248 | 32.307 | 38.880 | −7.780 | 1.00 | 0.00 | N |
| ATOM | 1830 | CZ | ARG | A 248 | 32.301 | 37.630 | −8.242 | 1.00 | 0.00 | C |
| ATOM | 1831 | NH1 | ARG | A 248 | 32.283 | 37.400 | −9.549 | 1.00 | 0.00 | N |
| ATOM | 1832 | NH2 | ARG | A 248 | 32.302 | 36.607 | −7.396 | 1.00 | 0.00 | N |
| ATOM | 1833 | N | GLN | A 249 | 34.532 | 44.156 | −8.506 | 1.00 | 0.00 | N |
| ATOM | 1834 | CA | GLN | A 249 | 34.353 | 45.143 | −9.562 | 1.00 | 0.00 | C |
| ATOM | 1835 | C | GLN | A 249 | 33.258 | 44.690 | −10.526 | 1.00 | 0.00 | C |
| ATOM | 1836 | O | GLN | A 249 | 33.052 | 43.492 | −10.729 | 1.00 | 0.00 | O |
| ATOM | 1837 | CB | GLN | A 249 | 35.681 | 45.364 | −10.297 | 1.00 | 0.00 | C |
| ATOM | 1838 | CG | GLN | A 249 | 36.810 | 45.825 | −9.372 | 1.00 | 0.00 | C |
| ATOM | 1839 | CD | GLN | A 249 | 36.443 | 47.082 | −8.593 | 1.00 | 0.00 | C |
| ATOM | 1840 | OE1 | GLN | A 249 | 36.462 | 47.096 | −7.356 | 1.00 | 0.00 | O |
| ATOM | 1841 | NE2 | GLN | A 249 | 36.103 | 48.144 | −9.314 | 1.00 | 0.00 | N |
| ATOM | 1842 | N | ILE | A 250 | 32.554 | 45.649 | −11.122 | 1.00 | 0.00 | N |
| ATOM | 1843 | CA | ILE | A 250 | 31.456 | 45.322 | −12.029 | 1.00 | 0.00 | C |
| ATOM | 1844 | C | ILE | A 250 | 31.785 | 44.404 | −13.208 | 1.00 | 0.00 | C |
| ATOM | 1845 | O | ILE | A 250 | 30.900 | 43.716 | −13.721 | 1.00 | 0.00 | O |
| ATOM | 1846 | CB | ILE | A 250 | 30.772 | 46.604 | −12.577 | 1.00 | 0.00 | C |
| ATOM | 1847 | CG1 | ILE | A 250 | 31.792 | 47.486 | −13.298 | 1.00 | 0.00 | C |
| ATOM | 1848 | CG2 | ILE | A 250 | 30.106 | 47.367 | −11.436 | 1.00 | 0.00 | C |
| ATOM | 1849 | CD1 | ILE | A 250 | 31.199 | 48.778 | −13.850 | 1.00 | 0.00 | C |
| ATOM | 1850 | N | TRP | A 251 | 33.047 | 44.373 | −13.625 | 1.00 | 0.00 | N |
| ATOM | 1851 | CA | TRP | A 251 | 33.457 | 43.541 | −14.759 | 1.00 | 0.00 | C |
| ATOM | 1852 | C | TRP | A 251 | 34.122 | 42.228 | −14.339 | 1.00 | 0.00 | C |
| ATOM | 1853 | O | TRP | A 251 | 34.455 | 41.395 | −15.187 | 1.00 | 0.00 | O |
| ATOM | 1854 | CB | TRP | A 251 | 34.445 | 44.316 | −15.625 | 1.00 | 0.00 | C |
| ATOM | 1855 | CG | TRP | A 251 | 35.745 | 44.505 | −14.922 | 1.00 | 0.00 | C |
| ATOM | 1856 | CD1 | TRP | A 251 | 36.736 | 43.576 | −14.760 | 1.00 | 0.00 | C |
| ATOM | 1857 | CD2 | TRP | A 251 | 36.158 | 45.662 | −14.196 | 1.00 | 0.00 | C |
| ATOM | 1858 | NE1 | TRP | A 251 | 37.736 | 44.082 | −13.973 | 1.00 | 0.00 | N |
| ATOM | 1859 | CE2 | TRP | A 251 | 37.407 | 45.362 | −13.610 | 1.00 | 0.00 | C |
| ATOM | 1860 | CE3 | TRP | A 251 | 35.590 | 46.921 | −13.975 | 1.00 | 0.00 | C |
| ATOM | 1861 | CZ2 | TRP | A 251 | 38.102 | 46.279 | −12.821 | 1.00 | 0.00 | C |
| ATOM | 1862 | CZ3 | TRP | A 251 | 36.278 | 47.834 | −13.191 | 1.00 | 0.00 | C |
| ATOM | 1863 | CH2 | TRP | A 251 | 37.523 | 47.506 | −12.621 | 1.00 | 0.00 | C |
| ATOM | 1864 | N | ASP | A 252 | 34.324 | 42.058 | −13.037 | 1.00 | 0.00 | N |
| ATOM | 1865 | CA | ASP | A 252 | 34.992 | 40.880 | −12.490 | 1.00 | 0.00 | C |
| ATOM | 1866 | C | ASP | A 252 | 34.118 | 39.626 | −12.430 | 1.00 | 0.00 | C |
| ATOM | 1867 | O | ASP | A 252 | 33.322 | 39.448 | −11.513 | 1.00 | 0.00 | O |
| ATOM | 1868 | CB | ASP | A 252 | 35.539 | 41.225 | −11.098 | 1.00 | 0.00 | C |
| ATOM | 1869 | CG | ASP | A 252 | 36.312 | 40.084 | −10.471 | 1.00 | 0.00 | C |
| ATOM | 1870 | OD1 | ASP | A 252 | 36.635 | 39.111 | −11.191 | 1.00 | 0.00 | O |
| ATOM | 1871 | OD2 | ASP | A 252 | 36.601 | 40.167 | −9.258 | 1.00 | 0.00 | O |
| ATOM | 1872 | N | ASN | A 253 | 34.287 | 38.750 | −13.415 | 1.00 | 0.00 | N |
| ATOM | 1873 | CA | ASN | A 253 | 33.508 | 37.520 | −13.489 | 1.00 | 0.00 | C |
| ATOM | 1874 | C | ASN | A 253 | 33.975 | 36.456 | −12.495 | 1.00 | 0.00 | C |
| ATOM | 1875 | O | ASN | A 253 | 33.158 | 35.745 | −11.909 | 1.00 | 0.00 | O |
| ATOM | 1876 | CB | ASN | A 253 | 33.579 | 36.958 | −14.910 | 1.00 | 0.00 | C |
| ATOM | 1877 | CG | ASN | A 253 | 32.722 | 35.728 | −15.091 | 1.00 | 0.00 | C |
| ATOM | 1878 | OD1 | ASN | A 253 | 33.196 | 34.695 | −15.561 | 1.00 | 0.00 | O |
| ATOM | 1879 | ND2 | ASN | A 253 | 31.449 | 35.832 | −14.726 | 1.00 | 0.00 | N |
| ATOM | 1880 | N | LYS | A 254 | 35.286 | 36.357 | −12.299 | 1.00 | 0.00 | N |
| ATOM | 1881 | CA | LYS | A 254 | 35.851 | 35.358 | −11.390 | 1.00 | 0.00 | C |
| ATOM | 1882 | C | LYS | A 254 | 35.733 | 35.750 | −9.918 | 1.00 | 0.00 | C |
| ATOM | 1883 | O | LYS | A 254 | 35.510 | 34.895 | −9.056 | 1.00 | 0.00 | O |
| ATOM | 1884 | CB | LYS | A 254 | 37.321 | 35.106 | −11.747 | 1.00 | 0.00 | C |
| ATOM | 1885 | CG | LYS | A 254 | 37.939 | 33.885 | −11.071 | 1.00 | 0.00 | C |
| ATOM | 1886 | CD | LYS | A 254 | 39.321 | 33.584 | −11.647 | 1.00 | 0.00 | C |
| ATOM | 1887 | CE | LYS | A 254 | 39.916 | 32.308 | −11.058 | 1.00 | 0.00 | C |
| ATOM | 1888 | NZ | LYS | A 254 | 40.119 | 32.400 | −9.584 | 1.00 | 0.00 | N |
| ATOM | 1889 | N | GLY | A 255 | 35.891 | 37.039 | −9.632 | 1.00 | 0.00 | N |
| ATOM | 1890 | CA | GLY | A 255 | 35.788 | 37.509 | −8.262 | 1.00 | 0.00 | C |
| ATOM | 1891 | C | GLY | A 255 | 37.108 | 37.716 | −7.535 | 1.00 | 0.00 | C |
| ATOM | 1892 | O | GLY | A 255 | 37.122 | 37.866 | −6.315 | 1.00 | 0.00 | O |
| ATOM | 1893 | N | ASP | A 256 | 38.216 | 37.737 | −8.270 | 1.00 | 0.00 | N |
| ATOM | 1894 | CA | ASP | A 256 | 39.525 | 37.923 | −7.647 | 1.00 | 0.00 | C |
| ATOM | 1895 | C | ASP | A 256 | 39.742 | 39.323 | −7.075 | 1.00 | 0.00 | C |
| ATOM | 1896 | O | ASP | A 256 | 40.667 | 39.537 | −6.296 | 1.00 | 0.00 | O |
| ATOM | 1897 | CB | ASP | A 256 | 40.648 | 37.616 | −8.644 | 1.00 | 0.00 | C |
| ATOM | 1898 | CG | ASP | A 256 | 40.647 | 36.171 | −9.100 | 1.00 | 0.00 | C |
| ATOM | 1899 | OD1 | ASP | A 256 | 40.503 | 35.273 | −8.240 | 1.00 | 0.00 | O |
| ATOM | 1900 | OD2 | ASP | A 256 | 40.801 | 35.934 | −10.317 | 1.00 | 0.00 | O |
| ATOM | 1901 | N | THR | A 257 | 38.900 | 40.276 | −7.460 | 1.00 | 0.00 | N |
| ATOM | 1902 | CA | THR | A 257 | 39.037 | 41.641 | −6.954 | 1.00 | 0.00 | C |
| ATOM | 1903 | C | THR | A 257 | 38.231 | 41.850 | −5.672 | 1.00 | 0.00 | C |
| ATOM | 1904 | O | THR | A 257 | 38.333 | 42.898 | −5.027 | 1.00 | 0.00 | O |
| ATOM | 1905 | CB | THR | A 257 | 38.545 | 42.670 | −7.982 | 1.00 | 0.00 | C |
| ATOM | 1906 | OG1 | THR | A 257 | 37.141 | 42.487 | −8.200 | 1.00 | 0.00 | O |
| ATOM | 1907 | CG2 | THR | A 257 | 39.290 | 42.506 | −9.299 | 1.00 | 0.00 | C |
| ATOM | 1908 | N | ALA | A 258 | 37.437 | 40.845 | −5.311 | 1.00 | 0.00 | N |
| ATOM | 1909 | CA | ALA | A 258 | 36.579 | 40.907 | −4.135 | 1.00 | 0.00 | C |
| ATOM | 1910 | C | ALA | A 258 | 37.266 | 41.290 | −2.828 | 1.00 | 0.00 | C |
| ATOM | 1911 | O | ALA | A 258 | 38.355 | 40.813 | −2.519 | 1.00 | 0.00 | O |
| ATOM | 1912 | CB | ALA | A 258 | 35.852 | 39.576 | −3.956 | 1.00 | 0.00 | C |
| ATOM | 1913 | N | LEU | A 259 | 36.600 | 42.154 | −2.066 | 1.00 | 0.00 | N |
| ATOM | 1914 | CA | LEU | A 259 | 37.091 | 42.605 | −0.767 | 1.00 | 0.00 | C |
| ATOM | 1915 | C | LEU | A 259 | 35.900 | 42.632 | 0.187 | 1.00 | 0.00 | C |
| ATOM | 1916 | O | LEU | A 259 | 34.874 | 43.245 | −0.110 | 1.00 | 0.00 | O |
| ATOM | 1917 | CB | LEU | A 259 | 37.700 | 44.009 | −0.866 | 1.00 | 0.00 | C |
| ATOM | 1918 | CG | LEU | A 259 | 38.382 | 44.511 | 0.410 | 1.00 | 0.00 | C |
| ATOM | 1919 | CD1 | LEU | A 259 | 39.566 | 43.601 | 0.743 | 1.00 | 0.00 | C |
| ATOM | 1920 | CD2 | LEU | A 259 | 38.849 | 45.957 | 0.226 | 1.00 | 0.00 | C |
| ATOM | 1921 | N | PHE | A 260 | 36.030 | 41.957 | 1.326 | 1.00 | 0.00 | N |
| ATOM | 1922 | CA | PHE | A 260 | 34.948 | 41.921 | 2.304 | 1.00 | 0.00 | C |
| ATOM | 1923 | C | PHE | A 260 | 34.625 | 43.336 | 2.774 | 1.00 | 0.00 | C |
| ATOM | 1924 | O | PHE | A 260 | 35.505 | 44.075 | 3.225 | 1.00 | 0.00 | O |
| ATOM | 1925 | CB | PHE | A 260 | 35.342 | 41.053 | 3.501 | 1.00 | 0.00 | C |
| ATOM | 1926 | CG | PHE | A 260 | 34.242 | 40.879 | 4.503 | 1.00 | 0.00 | C |
| ATOM | 1927 | CD1 | PHE | A 260 | 33.245 | 39.925 | 4.304 | 1.00 | 0.00 | C |
| ATOM | 1928 | CD2 | PHE | A 260 | 34.187 | 41.681 | 5.636 | 1.00 | 0.00 | C |
| ATOM | 1929 | CE1 | PHE | A 260 | 32.211 | 39.775 | 5.220 | 1.00 | 0.00 | C |
| ATOM | 1930 | CE2 | PHE | A 260 | 33.158 | 41.542 | 6.560 | 1.00 | 0.00 | C |
| ATOM | 1931 | CZ | PHE | A 260 | 32.166 | 40.588 | 6.353 | 1.00 | 0.00 | C |
| ATOM | 1932 | N | THR | A 261 | 33.355 | 43.708 | 2.680 | 1.00 | 0.00 | N |
| ATOM | 1933 | CA | THR | A 261 | 32.931 | 45.046 | 3.062 | 1.00 | 0.00 | C |
| ATOM | 1934 | C | THR | A 261 | 31.795 | 45.042 | 4.074 | 1.00 | 0.00 | C |
| ATOM | 1935 | O | THR | A 261 | 30.841 | 44.277 | 3.952 | 1.00 | 0.00 | O |
| ATOM | 1936 | CB | THR | A 261 | 32.463 | 45.837 | 1.824 | 1.00 | 0.00 | C |
| ATOM | 1937 | OG1 | THR | A 261 | 33.507 | 45.842 | 0.841 | 1.00 | 0.00 | O |
| ATOM | 1938 | CG2 | THR | A 261 | 32.117 | 47.277 | 2.198 | 1.00 | 0.00 | C |
| ATOM | 1939 | N | HIS | A 262 | 31.908 | 45.919 | 5.066 | 1.00 | 0.00 | N |
| ATOM | 1940 | CA | HIS | A 262 | 30.893 | 46.069 | 6.096 | 1.00 | 0.00 | C |
| ATOM | 1941 | C | HIS | A 262 | 30.269 | 47.450 | 5.946 | 1.00 | 0.00 | C |
| ATOM | 1942 | O | HIS | A 262 | 30.961 | 48.462 | 6.059 | 1.00 | 0.00 | O |
| ATOM | 1943 | CB | HIS | A 262 | 31.521 | 45.971 | 7.487 | 1.00 | 0.00 | C |
| ATOM | 1944 | CG | HIS | A 262 | 30.584 | 46.329 | 8.600 | 1.00 | 0.00 | C |
| ATOM | 1945 | ND1 | HIS | A 262 | 29.955 | 45.381 | 9.378 | 1.00 | 0.00 | N |
| ATOM | 1946 | CD2 | HIS | A 262 | 30.172 | 47.532 | 9.069 | 1.00 | 0.00 | C |
| ATOM | 1947 | CE1 | HIS | A 262 | 29.200 | 45.984 | 10.280 | 1.00 | 0.00 | C |
| ATOM | 1948 | NE2 | HIS | A 262 | 29.314 | 47.289 | 10.115 | 1.00 | 0.00 | N |
| ATOM | 1949 | N | MET | A 263 | 28.968 | 47.491 | 5.684 | 1.00 | 0.00 | N |
| ATOM | 1950 | CA | MET | A 263 | 28.269 | 48.760 | 5.556 | 1.00 | 0.00 | C |
| ATOM | 1951 | C | MET | A 263 | 27.521 | 48.991 | 6.863 | 1.00 | 0.00 | C |
| ATOM | 1952 | O | MET | A 263 | 26.763 | 48.125 | 7.308 | 1.00 | 0.00 | O |
| ATOM | 1953 | CB | MET | A 263 | 27.259 | 48.717 | 4.404 | 1.00 | 0.00 | C |
| ATOM | 1954 | CG | MET | A 263 | 26.485 | 50.025 | 4.205 | 1.00 | 0.00 | C |
| ATOM | 1955 | SD | MET | A 263 | 25.054 | 49.854 | 3.084 | 1.00 | 0.00 | S |
| ATOM | 1956 | CE | MET | A 263 | 25.878 | 49.605 | 1.504 | 1.00 | 0.00 | C |
| ATOM | 1957 | N | MET | A 264 | 27.745 | 50.141 | 7.490 | 1.00 | 0.00 | N |
| ATOM | 1958 | CA | MET | A 264 | 27.043 | 50.451 | 8.728 | 1.00 | 0.00 | C |
| ATOM | 1959 | C | MET | A 264 | 25.579 | 50.606 | 8.322 | 1.00 | 0.00 | C |
| ATOM | 1960 | O | MET | A 264 | 25.280 | 51.049 | 7.213 | 1.00 | 0.00 | O |
| ATOM | 1961 | CB | MET | A 264 | 27.635 | 51.710 | 9.364 | 1.00 | 0.00 | C |
| ATOM | 1962 | CG | MET | A 264 | 29.037 | 51.446 | 9.918 | 1.00 | 0.00 | C |
| ATOM | 1963 | SD | MET | A 264 | 29.959 | 52.912 | 10.446 | 1.00 | 0.00 | S |
| ATOM | 1964 | CE | MET | A 264 | 28.964 | 53.477 | 11.815 | 1.00 | 0.00 | C |
| ATOM | 1965 | N | PRO | A 265 | 24.646 | 50.244 | 9.214 | 1.00 | 0.00 | N |
| ATOM | 1966 | CA | PRO | A 265 | 23.211 | 50.315 | 8.928 | 1.00 | 0.00 | C |
| ATOM | 1967 | C | PRO | A 265 | 22.429 | 51.590 | 9.187 | 1.00 | 0.00 | C |
| ATOM | 1968 | O | PRO | A 265 | 21.340 | 51.770 | 8.634 | 1.00 | 0.00 | O |
| ATOM | 1969 | CB | PRO | A 265 | 22.664 | 49.172 | 9.767 | 1.00 | 0.00 | C |
| ATOM | 1970 | CG | PRO | A 265 | 23.451 | 49.352 | 11.044 | 1.00 | 0.00 | C |
| ATOM | 1971 | CD | PRO | A 265 | 24.879 | 49.679 | 10.558 | 1.00 | 0.00 | C |
| ATOM | 1972 | N | PHE | A 266 | 22.974 | 52.480 | 10.000 | 1.00 | 0.00 | N |
| ATOM | 1973 | CA | PHE | A 266 | 22.229 | 53.668 | 10.362 | 1.00 | 0.00 | C |
| ATOM | 1974 | C | PHE | A 266 | 22.601 | 55.022 | 9.747 | 1.00 | 0.00 | C |
| ATOM | 1975 | O | PHE | A 266 | 23.522 | 55.139 | 8.937 | 1.00 | 0.00 | O |
| ATOM | 1976 | CB | PHE | A 266 | 22.188 | 53.722 | 11.892 | 1.00 | 0.00 | C |
| ATOM | 1977 | CG | PHE | A 266 | 21.653 | 52.446 | 12.521 | 1.00 | 0.00 | C |
| ATOM | 1978 | CD1 | PHE | A 266 | 22.244 | 51.906 | 13.661 | 1.00 | 0.00 | C |
| ATOM | 1979 | CD2 | PHE | A 266 | 20.559 | 51.785 | 11.959 | 1.00 | 0.00 | C |
| ATOM | 1980 | CE1 | PHE | A 266 | 21.752 | 50.724 | 14.234 | 1.00 | 0.00 | C |
| ATOM | 1981 | CE2 | PHE | A 266 | 20.058 | 50.604 | 12.522 | 1.00 | 0.00 | C |
| ATOM | 1982 | CZ | PHE | A 266 | 20.655 | 50.072 | 13.660 | 1.00 | 0.00 | C |
| ATOM | 1983 | N | TYR | A 267 | 21.838 | 56.034 | 10.145 | 1.00 | 0.00 | N |
| ATOM | 1984 | CA | TYR | A 267 | 21.953 | 57.410 | 9.659 | 1.00 | 0.00 | C |
| ATOM | 1985 | C | TYR | A 267 | 23.291 | 58.117 | 9.865 | 1.00 | 0.00 | C |
| ATOM | 1986 | O | TYR | A 267 | 23.696 | 58.941 | 9.040 | 1.00 | 0.00 | O |
| ATOM | 1987 | CB | TYR | A 267 | 20.828 | 58.229 | 10.301 | 1.00 | 0.00 | C |
| ATOM | 1988 | CG | TYR | A 267 | 20.881 | 59.726 | 10.097 | 1.00 | 0.00 | C |
| ATOM | 1989 | CD1 | TYR | A 267 | 20.521 | 60.304 | 8.882 | 1.00 | 0.00 | C |
| ATOM | 1990 | CD2 | TYR | A 267 | 21.244 | 60.570 | 11.147 | 1.00 | 0.00 | C |
| ATOM | 1991 | CE1 | TYR | A 267 | 20.513 | 61.698 | 8.719 | 1.00 | 0.00 | C |
| ATOM | 1992 | CE2 | TYR | A 267 | 21.243 | 61.954 | 10.995 | 1.00 | 0.00 | C |
| ATOM | 1993 | CZ | TYR | A 267 | 20.875 | 62.510 | 9.785 | 1.00 | 0.00 | C |
| ATOM | 1994 | OH | TYR | A 267 | 20.857 | 63.879 | 9.651 | 1.00 | 0.00 | O |
| ATOM | 1995 | N | SER | A 268 | 23.976 | 57.803 | 10.956 | 1.00 | 0.00 | N |
| ATOM | 1996 | CA | SER | A 268 | 25.245 | 58.459 | 11.243 | 1.00 | 0.00 | C |
| ATOM | 1997 | C | SER | A 268 | 26.227 | 57.552 | 11.973 | 1.00 | 0.00 | C |
| ATOM | 1998 | O | SER | A 268 | 25.868 | 56.461 | 12.425 | 1.00 | 0.00 | O |
| ATOM | 1999 | CB | SER | A 268 | 24.977 | 59.717 | 12.077 | 1.00 | 0.00 | C |
| ATOM | 2000 | OG | SER | A 268 | 26.170 | 60.276 | 12.592 | 1.00 | 0.00 | O |
| ATOM | 2001 | N | TYR | A 269 | 27.474 | 58.007 | 12.076 | 1.00 | 0.00 | N |
| ATOM | 2002 | CA | TYR | A 269 | 28.500 | 57.252 | 12.778 | 1.00 | 0.00 | C |
| ATOM | 2003 | C | TYR | A 269 | 28.622 | 57.740 | 14.221 | 1.00 | 0.00 | C |
| ATOM | 2004 | O | TYR | A 269 | 29.445 | 57.228 | 14.978 | 1.00 | 0.00 | O |
| ATOM | 2005 | CB | TYR | A 269 | 29.857 | 57.386 | 12.069 | 1.00 | 0.00 | C |
| ATOM | 2006 | CG | TYR | A 269 | 30.306 | 58.815 | 11.846 | 1.00 | 0.00 | C |
| ATOM | 2007 | CD1 | TYR | A 269 | 30.721 | 59.618 | 12.911 | 1.00 | 0.00 | C |
| ATOM | 2008 | CD2 | TYR | A 269 | 30.287 | 59.374 | 10.567 | 1.00 | 0.00 | C |
| ATOM | 2009 | CE1 | TYR | A 269 | 31.104 | 60.950 | 12.708 | 1.00 | 0.00 | C |
| ATOM | 2010 | CE2 | TYR | A 269 | 30.665 | 60.701 | 10.352 | 1.00 | 0.00 | C |
| ATOM | 2011 | CZ | TYR | A 269 | 31.068 | 61.482 | 11.422 | 1.00 | 0.00 | C |
| ATOM | 2012 | OH | TYR | A 269 | 31.402 | 62.800 | 11.205 | 1.00 | 0.00 | O |
| ATOM | 2013 | N | ASP | A 270 | 27.817 | 58.731 | 14.605 | 1.00 | 0.00 | N |
| ATOM | 2014 | CA | ASP | A 270 | 27.893 | 59.225 | 15.980 | 1.00 | 0.00 | C |
| ATOM | 2015 | C | ASP | A 270 | 27.310 | 58.186 | 16.933 | 1.00 | 0.00 | C |
| ATOM | 2016 | O | ASP | A 270 | 26.658 | 57.236 | 16.501 | 1.00 | 0.00 | O |
| ATOM | 2017 | CB | ASP | A 270 | 27.196 | 60.591 | 16.144 | 1.00 | 0.00 | C |
| ATOM | 2018 | CG | ASP | A 270 | 25.702 | 60.554 | 15.860 | 1.00 | 0.00 | C |
| ATOM | 2019 | OD1 | ASP | A 270 | 25.104 | 59.457 | 15.796 | 1.00 | 0.00 | O |
| ATOM | 2020 | OD2 | ASP | A 270 | 25.120 | 61.655 | 15.721 | 1.00 | 0.00 | O |
| ATOM | 2021 | N | ILE | A 271 | 27.553 | 58.351 | 18.226 | 1.00 | 0.00 | N |
| ATOM | 2022 | CA | ILE | A 271 | 27.080 | 57.366 | 19.187 | 1.00 | 0.00 | C |
| ATOM | 2023 | C | ILE | A 271 | 25.567 | 57.124 | 19.164 | 1.00 | 0.00 | C |
| ATOM | 2024 | O | ILE | A 271 | 25.124 | 55.979 | 19.239 | 1.00 | 0.00 | O |
| ATOM | 2025 | CB | ILE | A 271 | 27.585 | 57.718 | 20.606 | 1.00 | 0.00 | C |
| ATOM | 2026 | CG1 | ILE | A 271 | 29.119 | 57.658 | 20.612 | 1.00 | 0.00 | C |
| ATOM | 2027 | CG2 | ILE | A 271 | 27.045 | 56.718 | 21.628 | 1.00 | 0.00 | C |
| ATOM | 2028 | CD1 | ILE | A 271 | 29.776 | 58.225 | 21.863 | 1.00 | 0.00 | C |
| ATOM | 2029 | N | PRO | A 272 | 24.755 | 58.187 | 19.043 | 1.00 | 0.00 | N |
| ATOM | 2030 | CA | PRO | A 272 | 23.306 | 57.961 | 19.013 | 1.00 | 0.00 | C |
| ATOM | 2031 | C | PRO | A 272 | 22.861 | 57.025 | 17.881 | 1.00 | 0.00 | C |
| ATOM | 2032 | O | PRO | A 272 | 21.816 | 56.387 | 17.979 | 1.00 | 0.00 | O |
| ATOM | 2033 | CB | PRO | A 272 | 22.740 | 59.369 | 18.856 | 1.00 | 0.00 | C |
| ATOM | 2034 | CG | PRO | A 272 | 23.741 | 60.208 | 19.599 | 1.00 | 0.00 | C |
| ATOM | 2035 | CD | PRO | A 272 | 25.059 | 59.627 | 19.135 | 1.00 | 0.00 | C |
| ATOM | 2036 | N | HIS | A 273 | 23.653 | 56.937 | 16.812 | 1.00 | 0.00 | N |
| ATOM | 2037 | CA | HIS | A 273 | 23.301 | 56.075 | 15.682 | 1.00 | 0.00 | C |
| ATOM | 2038 | C | HIS | A 273 | 24.229 | 54.880 | 15.459 | 1.00 | 0.00 | C |
| ATOM | 2039 | O | HIS | A 273 | 24.338 | 54.373 | 14.338 | 1.00 | 0.00 | O |
| ATOM | 2040 | CB | HIS | A 273 | 23.217 | 56.901 | 14.391 | 1.00 | 0.00 | C |
| ATOM | 2041 | CG | HIS | A 273 | 22.162 | 57.964 | 14.428 | 1.00 | 0.00 | C |
| ATOM | 2042 | ND1 | HIS | A 273 | 22.362 | 59.193 | 15.019 | 1.00 | 0.00 | N |
| ATOM | 2043 | CD2 | HIS | A 273 | 20.879 | 57.962 | 13.993 | 1.00 | 0.00 | C |
| ATOM | 2044 | CE1 | HIS | A 273 | 21.249 | 59.901 | 14.949 | 1.00 | 0.00 | C |
| ATOM | 2045 | NE2 | HIS | A 273 | 20.333 | 59.176 | 14.330 | 1.00 | 0.00 | N |
| ATOM | 2046 | N | THR | A 274 | 24.887 | 54.419 | 16.520 | 1.00 | 0.00 | N |
| ATOM | 2047 | CA | THR | A 274 | 25.783 | 53.276 | 16.394 | 1.00 | 0.00 | C |
| ATOM | 2048 | C | THR | A 274 | 25.588 | 52.156 | 17.423 | 1.00 | 0.00 | C |
| ATOM | 2049 | O | THR | A 274 | 26.159 | 51.078 | 17.268 | 1.00 | 0.00 | O |
| ATOM | 2050 | CB | THR | A 274 | 27.269 | 53.727 | 16.409 | 1.00 | 0.00 | C |
| ATOM | 2051 | OG1 | THR | A 274 | 27.480 | 54.679 | 17.456 | 1.00 | 0.00 | O |
| ATOM | 2052 | OG2 | THR | A 274 | 27.646 | 54.350 | 15.074 | 1.00 | 0.00 | C |
| ATOM | 2053 | N | CYS | A 275 | 24.786 | 52.388 | 18.462 | 1.00 | 0.00 | N |
| ATOM | 2054 | CA | CYS | A 275 | 24.564 | 51.344 | 19.468 | 1.00 | 0.00 | C |
| ATOM | 2055 | C | CYS | A 275 | 23.375 | 50.457 | 19.108 | 1.00 | 0.00 | C |
| ATOM | 2056 | O | CYS | A 275 | 23.299 | 49.296 | 19.521 | 1.00 | 0.00 | O |
| ATOM | 2057 | CB | CYS | A 275 | 24.327 | 51.967 | 20.848 | 1.00 | 0.00 | C |
| ATOM | 2058 | SG | CYS | A 275 | 22.589 | 52.094 | 21.403 | 1.00 | 0.00 | S |
| ATOM | 2059 | N | GLY | A 276 | 22.450 | 51.016 | 18.338 | 1.00 | 0.00 | N |
| ATOM | 2060 | CA | GLY | A 276 | 21.260 | 50.288 | 17.941 | 1.00 | 0.00 | C |
| ATOM | 2061 | C | GLY | A 276 | 20.342 | 51.180 | 17.126 | 1.00 | 0.00 | C |
| ATOM | 2062 | O | GLY | A 276 | 20.715 | 52.314 | 16.817 | 1.00 | 0.00 | O |
| ATOM | 2063 | N | PRO | A 277 | 19.130 | 50.709 | 16.778 | 1.00 | 0.00 | N |
| ATOM | 2064 | CA | PRO | A 277 | 18.130 | 51.438 | 15.988 | 1.00 | 0.00 | C |
| ATOM | 2065 | C | PRO | A 277 | 17.466 | 52.657 | 16.624 | 1.00 | 0.00 | C |
| ATOM | 2066 | O | PRO | A 277 | 16.883 | 53.477 | 15.916 | 1.00 | 0.00 | O |
| ATOM | 2067 | CB | PRO | A 277 | 17.103 | 50.356 | 15.662 | 1.00 | 0.00 | C |
| ATOM | 2068 | CG | PRO | A 277 | 17.124 | 49.525 | 16.904 | 1.00 | 0.00 | C |
| ATOM | 2069 | CD | PRO | A 277 | 18.610 | 49.384 | 17.165 | 1.00 | 0.00 | C |
| ATOM | 2070 | N | ASP | A 278 | 17.543 | 52.782 | 17.946 | 1.00 | 0.00 | N |
| ATOM | 2071 | CA | ASP | A 278 | 16.896 | 53.903 | 18.630 | 1.00 | 0.00 | C |
| ATOM | 2072 | C | ASP | A 278 | 17.872 | 54.940 | 19.175 | 1.00 | 0.00 | C |
| ATOM | 2073 | O | ASP | A 278 | 18.487 | 54.739 | 20.221 | 1.00 | 0.00 | O |
| ATOM | 2074 | CB | ASP | A 278 | 16.027 | 53.383 | 19.781 | 1.00 | 0.00 | C |
| ATOM | 2075 | CG | ASP | A 278 | 15.096 | 54.448 | 20.335 | 1.00 | 0.00 | C |
| ATOM | 2076 | OD1 | ASP | A 278 | 15.253 | 55.634 | 19.970 | 1.00 | 0.00 | O |
| ATOM | 2077 | OD2 | ASP | A 278 | 14.209 | 54.098 | 21.138 | 1.00 | 0.00 | O |
| ATOM | 2078 | N | PRO | A 279 | 18.011 | 56.077 | 18.478 | 1.00 | 0.00 | N |
| ATOM | 2079 | CA | PRO | A 279 | 18.927 | 57.132 | 18.925 | 1.00 | 0.00 | C |
| ATOM | 2080 | C | PRO | A 279 | 18.575 | 57.727 | 20.289 | 1.00 | 0.00 | C |
| ATOM | 2081 | O | PRO | A 279 | 19.450 | 58.222 | 20.997 | 1.00 | 0.00 | O |
| ATOM | 2082 | CB | PRO | A 279 | 18.851 | 58.159 | 17.792 | 1.00 | 0.00 | C |
| ATOM | 2083 | CG | PRO | A 279 | 17.461 | 57.974 | 17.260 | 1.00 | 0.00 | C |
| ATOM | 2084 | CD | PRO | A 279 | 17.323 | 56.470 | 17.235 | 1.00 | 0.00 | C |
| ATOM | 2085 | N | LYS | A 280 | 17.299 | 57.678 | 20.662 | 1.00 | 0.00 | N |
| ATOM | 2086 | CA | LYS | A 280 | 16.886 | 58.217 | 21.954 | 1.00 | 0.00 | C |
| ATOM | 2087 | C | LYS | A 280 | 17.499 | 57.389 | 23.077 | 1.00 | 0.00 | C |
| ATOM | 2088 | O | LYS | A 280 | 17.776 | 57.901 | 24.163 | 1.00 | 0.00 | O |
| ATOM | 2089 | CB | LYS | A 280 | 15.361 | 58.211 | 22.084 | 1.00 | 0.00 | C |
| ATOM | 2090 | CG | LYS | A 280 | 14.861 | 58.895 | 23.350 | 1.00 | 0.00 | C |
| ATOM | 2091 | CD | LYS | A 280 | 13.348 | 58.830 | 23.464 | 1.00 | 0.00 | C |
| ATOM | 2092 | CE | LYS | A 280 | 12.876 | 57.405 | 23.700 | 1.00 | 0.00 | C |
| ATOM | 2093 | NZ | LYS | A 280 | 13.443 | 56.828 | 24.961 | 1.00 | 0.00 | N |
| ATOM | 2094 | N | VAL | A 281 | 17.708 | 56.104 | 22.813 | 1.00 | 0.00 | N |
| ATOM | 2095 | CA | VAL | A 281 | 18.306 | 55.217 | 23.804 | 1.00 | 0.00 | C |
| ATOM | 2096 | C | VAL | A 281 | 19.831 | 55.282 | 23.699 | 1.00 | 0.00 | C |
| ATOM | 2097 | O | VAL | A 281 | 20.530 | 55.439 | 24.699 | 1.00 | 0.00 | O |
| ATOM | 2098 | CB | VAL | A 281 | 17.850 | 53.751 | 23.594 | 1.00 | 0.00 | C |
| ATOM | 2099 | CG1 | VAL | A 281 | 18.589 | 52.829 | 24.555 | 1.00 | 0.00 | C |
| ATOM | 2100 | CG2 | VAL | A 281 | 16.341 | 53.636 | 23.801 | 1.00 | 0.00 | C |
| ATOM | 2101 | N | CYS | A 282 | 20.344 | 55.166 | 22.478 | 1.00 | 0.00 | N |
| ATOM | 2102 | CA | CYS | A 282 | 21.786 | 55.201 | 22.261 | 1.00 | 0.00 | C |
| ATOM | 2103 | C | CYS | A 282 | 22.430 | 56.486 | 22.768 | 1.00 | 0.00 | C |
| ATOM | 2104 | O | CYS | A 282 | 23.555 | 56.468 | 23.277 | 1.00 | 0.00 | O |
| ATOM | 2105 | CB | CYS | A 282 | 22.100 | 55.031 | 20.775 | 1.00 | 0.00 | C |
| ATOM | 2106 | SG | CYS | A 282 | 21.727 | 53.385 | 20.093 | 1.00 | 0.00 | S |
| ATOM | 2107 | N | CYS | A 283 | 21.721 | 57.603 | 22.638 | 1.00 | 0.00 | N |
| ATOM | 2108 | CA | CYS | A 283 | 22.271 | 58.873 | 23.086 | 1.00 | 0.00 | C |
| ATOM | 2109 | C | CYS | A 283 | 22.577 | 58.834 | 24.579 | 1.00 | 0.00 | C |
| ATOM | 2110 | O | CYS | A 283 | 23.485 | 59.515 | 25.055 | 1.00 | 0.00 | O |
| ATOM | 2111 | CB | CYS | A 283 | 21.307 | 60.025 | 22.785 | 1.00 | 0.00 | C |
| ATOM | 2112 | SG | CYS | A 283 | 22.167 | 61.630 | 22.843 | 1.00 | 0.00 | S |
| ATOM | 2113 | N | GLN | A 284 | 21.821 | 58.025 | 25.314 | 1.00 | 0.00 | N |
| ATOM | 2114 | CA | GLN | A 284 | 22.015 | 57.904 | 26.752 | 1.00 | 0.00 | C |
| ATOM | 2115 | C | GLN | A 284 | 23.300 | 57.173 | 27.093 | 1.00 | 0.00 | C |
| ATOM | 2116 | O | GLN | A 284 | 23.679 | 57.080 | 28.261 | 1.00 | 0.00 | O |
| ATOM | 2117 | CB | GLN | A 284 | 20.824 | 57.180 | 27.381 | 1.00 | 0.00 | C |
| ATOM | 2118 | CG | GLN | A 284 | 19.513 | 57.927 | 27.217 | 1.00 | 0.00 | C |
| ATOM | 2119 | CD | GLN | A 284 | 18.333 | 57.138 | 27.732 | 1.00 | 0.00 | C |
| ATOM | 2120 | OE1 | GLN | A 284 | 18.284 | 56.765 | 28.905 | 1.00 | 0.00 | O |
| ATOM | 2121 | NE2 | GLN | A 284 | 17.372 | 56.876 | 26.855 | 1.00 | 0.00 | N |
| ATOM | 2122 | N | PHE | A 285 | 23.978 | 56.657 | 26.074 | 1.00 | 0.00 | N |
| ATOM | 2123 | CA | PHE | A 285 | 25.215 | 55.941 | 26.309 | 1.00 | 0.00 | C |
| ATOM | 2124 | C | PHE | A 285 | 26.439 | 56.640 | 25.730 | 1.00 | 0.00 | C |
| ATOM | 2125 | O | PHE | A 285 | 27.480 | 56.026 | 25.500 | 1.00 | 0.00 | O |
| ATOM | 2126 | CB | PHE | A 285 | 25.061 | 54.498 | 25.820 | 1.00 | 0.00 | C |
| ATOM | 2127 | CG | PHE | A 285 | 24.076 | 53.709 | 26.645 | 1.00 | 0.00 | C |
| ATOM | 2128 | CD1 | PHE | A 285 | 24.474 | 53.103 | 27.833 | 1.00 | 0.00 | C |
| ATOM | 2129 | CD2 | PHE | A 285 | 22.732 | 53.652 | 26.285 | 1.00 | 0.00 | C |
| ATOM | 2130 | CE1 | PHE | A 285 | 23.549 | 52.454 | 28.657 | 1.00 | 0.00 | C |
| ATOM | 2131 | CE2 | PHE | A 285 | 21.795 | 53.006 | 27.101 | 1.00 | 0.00 | C |
| ATOM | 2132 | CZ | PHE | A 285 | 22.208 | 52.407 | 28.291 | 1.00 | 0.00 | C |
| ATOM | 2133 | N | ASP | A 286 | 26.283 | 57.942 | 25.498 | 1.00 | 0.00 | N |
| ATOM | 2134 | CA | ASP | A 286 | 27.367 | 58.806 | 25.041 | 1.00 | 0.00 | C |
| ATOM | 2135 | C | ASP | A 286 | 27.545 | 59.652 | 26.296 | 1.00 | 0.00 | C |
| ATOM | 2136 | O | ASP | A 286 | 26.904 | 60.688 | 26.462 | 1.00 | 0.00 | O |
| ATOM | 2137 | CB | ASP | A 286 | 26.930 | 59.693 | 23.874 | 1.00 | 0.00 | C |
| ATOM | 2138 | CG | ASP | A 286 | 28.047 | 60.605 | 23.391 | 1.00 | 0.00 | C |
| ATOM | 2139 | OD1 | ASP | A 286 | 29.087 | 60.686 | 24.079 | 1.00 | 0.00 | O |
| ATOM | 2140 | OD2 | ASP | A 286 | 27.884 | 61.248 | 22.331 | 1.00 | 0.00 | O |
| ATOM | 2141 | N | PHE | A 287 | 28.409 | 59.194 | 27.191 | 1.00 | 0.00 | N |
| ATOM | 2142 | CA | PHE | A 287 | 28.601 | 59.879 | 28.454 | 1.00 | 0.00 | C |
| ATOM | 2143 | C | PHE | A 287 | 29.236 | 61.263 | 28.424 | 1.00 | 0.00 | C |
| ATOM | 2144 | O | PHE | A 287 | 29.478 | 61.855 | 29.469 | 1.00 | 0.00 | O |
| ATOM | 2145 | CB | PHE | A 287 | 29.331 | 58.946 | 29.419 | 1.00 | 0.00 | C |
| ATOM | 2146 | CG | PHE | A 287 | 28.591 | 57.654 | 29.657 | 1.00 | 0.00 | C |
| ATOM | 2147 | CD1 | PHE | A 287 | 28.815 | 56.544 | 28.847 | 1.00 | 0.00 | C |
| ATOM | 2148 | CD2 | PHE | A 287 | 27.605 | 57.575 | 30.638 | 1.00 | 0.00 | C |
| ATOM | 2149 | CE1 | PHE | A 287 | 28.066 | 55.379 | 29.007 | 1.00 | 0.00 | C |
| ATOM | 2150 | CE2 | PHE | A 287 | 26.849 | 56.414 | 30.805 | 1.00 | 0.00 | C |
| ATOM | 2151 | CZ | PHE | A 287 | 27.080 | 55.313 | 29.986 | 1.00 | 0.00 | C |
| ATOM | 2152 | N | LYS | A 288 | 29.490 | 61.791 | 27.230 | 1.00 | 0.00 | N |
| ATOM | 2153 | CA | LYS | A 288 | 30.052 | 63.133 | 27.133 | 1.00 | 0.00 | C |
| ATOM | 2154 | C | LYS | A 288 | 28.900 | 64.136 | 27.025 | 1.00 | 0.00 | C |
| ATOM | 2155 | O | LYS | A 288 | 29.122 | 65.341 | 26.963 | 1.00 | 0.00 | O |
| ATOM | 2156 | CB | LYS | A 288 | 30.964 | 63.262 | 25.901 | 1.00 | 0.00 | C |
| ATOM | 2157 | CG | LYS | A 288 | 31.708 | 64.601 | 25.832 | 1.00 | 0.00 | C |
| ATOM | 2158 | CD | LYS | A 288 | 32.650 | 64.698 | 24.634 | 1.00 | 0.00 | C |
| ATOM | 2159 | CE | LYS | A 288 | 33.439 | 66.003 | 24.683 | 1.00 | 0.00 | C |
| ATOM | 2160 | NZ | LYS | A 288 | 34.384 | 66.172 | 23.531 | 1.00 | 0.00 | N |
| ATOM | 2161 | N | ARG | A 289 | 27.666 | 63.638 | 27.022 | 1.00 | 0.00 | N |
| ATOM | 2162 | CA | ARG | A 289 | 26.507 | 64.517 | 26.890 | 1.00 | 0.00 | C |
| ATOM | 2163 | C | ARG | A 289 | 25.711 | 64.768 | 28.172 | 1.00 | 0.00 | C |
| ATOM | 2164 | O | ARG | A 289 | 24.487 | 64.873 | 28.121 | 1.00 | 0.00 | O |
| ATOM | 2165 | CB | ARG | A 289 | 25.548 | 63.968 | 25.828 | 1.00 | 0.00 | C |
| ATOM | 2166 | CG | ARG | A 289 | 26.170 | 63.712 | 24.461 | 1.00 | 0.00 | C |
| ATOM | 2167 | CD | ARG | A 289 | 25.088 | 63.468 | 23.427 | 1.00 | 0.00 | C |
| ATOM | 2168 | NE | ARG | A 289 | 25.617 | 63.061 | 22.127 | 1.00 | 0.00 | N |
| ATOM | 2169 | CZ | ARG | A 289 | 25.139 | 63.493 | 20.965 | 1.00 | 0.00 | C |
| ATOM | 2170 | NH1 | ARG | A 289 | 24.128 | 64.355 | 20.942 | 1.00 | 0.00 | N |
| ATOM | 2171 | NH2 | ARG | A 289 | 25.656 | 63.052 | 19.826 | 1.00 | 0.00 | N |
| ATOM | 2172 | N | MET | A 290 | 26.382 | 64.885 | 29.313 | 1.00 | 0.00 | N |
| ATOM | 2173 | CA | MET | A 290 | 25.652 | 65.113 | 30.557 | 1.00 | 0.00 | C |
| ATOM | 2174 | C | MET | A 290 | 25.566 | 66.574 | 31.008 | 1.00 | 0.00 | C |
| ATOM | 2175 | O | MET | A 290 | 24.856 | 66.884 | 31.970 | 1.00 | 0.00 | O |
| ATOM | 2176 | CB | MET | A 290 | 26.226 | 64.236 | 31.676 | 1.00 | 0.00 | C |
| ATOM | 2177 | CG | MET | A 290 | 26.001 | 62.746 | 31.437 | 1.00 | 0.00 | C |
| ATOM | 2178 | SD | MET | A 290 | 26.499 | 61.651 | 32.789 | 1.00 | 0.00 | S |
| ATOM | 2179 | CE | MET | A 290 | 28.245 | 61.429 | 32.430 | 1.00 | 0.00 | C |
| ATOM | 2180 | N | GLY | A 291 | 26.275 | 67.470 | 30.320 | 1.00 | 0.00 | N |
| ATOM | 2181 | CA | GLY | A 291 | 26.209 | 68.878 | 30.684 | 1.00 | 0.00 | C |
| ATOM | 2182 | C | GLY | A 291 | 27.497 | 69.686 | 30.712 | 1.00 | 0.00 | C |
| ATOM | 2183 | O | GLY | A 291 | 27.628 | 70.676 | 29.987 | 1.00 | 0.00 | O |
| ATOM | 2184 | N | SER | A 292 | 28.444 | 69.274 | 31.551 | 1.00 | 0.00 | N |
| ATOM | 2185 | CA | SER | A 292 | 29.714 | 69.981 | 31.693 | 1.00 | 0.00 | C |
| ATOM | 2186 | C | SER | A 292 | 30.514 | 70.153 | 30.400 | 1.00 | 0.00 | C |
| ATOM | 2187 | O | SER | A 292 | 31.384 | 71.023 | 30.324 | 1.00 | 0.00 | O |
| ATOM | 2188 | CB | SER | A 292 | 30.588 | 69.284 | 32.736 | 1.00 | 0.00 | C |
| ATOM | 2189 | OG | SER | A 292 | 30.964 | 67.989 | 32.302 | 1.00 | 0.00 | O |
| ATOM | 2190 | N | PHE | A 293 | 30.231 | 69.329 | 29.393 | 1.00 | 0.00 | N |
| ATOM | 2191 | CA | PHE | A 293 | 30.944 | 69.418 | 28.119 | 1.00 | 0.00 | C |
| ATOM | 2192 | C | PHE | A 293 | 30.206 | 70.273 | 27.093 | 1.00 | 0.00 | C |
| ATOM | 2193 | O | PHE | A 293 | 30.664 | 70.428 | 25.957 | 1.00 | 0.00 | O |
| ATOM | 2194 | CB | PHE | A 293 | 31.178 | 68.023 | 27.525 | 1.00 | 0.00 | C |
| ATOM | 2195 | CG | PHE | A 293 | 32.115 | 67.171 | 28.329 | 1.00 | 0.00 | C |
| ATOM | 2196 | CD1 | PHE | A 293 | 31.628 | 66.289 | 29.285 | 1.00 | 0.00 | C |
| ATOM | 2197 | CD2 | PHE | A 293 | 33.489 | 67.254 | 28.132 | 1.00 | 0.00 | C |
| ATOM | 2198 | CE1 | PHE | A 293 | 32.496 | 65.498 | 30.035 | 1.00 | 0.00 | C |
| ATOM | 2199 | CE2 | PHE | A 293 | 34.369 | 66.470 | 28.875 | 1.00 | 0.00 | C |
| ATOM | 2200 | CZ | PHE | A 293 | 33.871 | 65.589 | 29.829 | 1.00 | 0.00 | C |
| ATOM | 2201 | N | GLY | A 294 | 29.064 | 70.823 | 27.492 | 1.00 | 0.00 | N |
| ATOM | 2202 | CA | GLY | A 294 | 28.294 | 71.652 | 26.582 | 1.00 | 0.00 | C |
| ATOM | 2203 | C | GLY | A 294 | 27.536 | 70.859 | 25.532 | 1.00 | 0.00 | C |
| ATOM | 2204 | O | GLY | A 294 | 27.094 | 71.414 | 24.524 | 1.00 | 0.00 | O |
| ATOM | 2205 | N | LEU | A 295 | 27.387 | 69.558 | 25.762 | 1.00 | 0.00 | N |
| ATOM | 2206 | CA | LEU | A 295 | 26.670 | 68.692 | 24.833 | 1.00 | 0.00 | C |
| ATOM | 2207 | C | LEU | A 295 | 25.448 | 68.107 | 25.530 | 1.00 | 0.00 | C |
| ATOM | 2208 | O | LEU | A 295 | 25.416 | 67.999 | 26.753 | 1.00 | 0.00 | O |
| ATOM | 2209 | CB | LEU | A 295 | 27.577 | 67.554 | 24.351 | 1.00 | 0.00 | C |
| ATOM | 2210 | CG | LEU | A 295 | 28.842 | 67.930 | 23.573 | 1.00 | 0.00 | C |
| ATOM | 2211 | CD1 | LEU | A 295 | 29.680 | 66.683 | 23.325 | 1.00 | 0.00 | C |
| ATOM | 2212 | CD2 | LEU | A 295 | 28.458 | 68.592 | 22.251 | 1.00 | 0.00 | C |
| ATOM | 2213 | N | SER | A 296 | 24.444 | 67.733 | 24.746 | 1.00 | 0.00 | N |
| ATOM | 2214 | CA | SER | A 296 | 23.226 | 67.152 | 25.297 | 1.00 | 0.00 | C |
| ATOM | 2215 | C | SER | A 296 | 22.563 | 66.263 | 24.257 | 1.00 | 0.00 | C |
| ATOM | 2216 | O | SER | A 296 | 23.024 | 66.175 | 23.116 | 1.00 | 0.00 | O |
| ATOM | 2217 | CB | SER | A 296 | 22.255 | 68.256 | 25.729 | 1.00 | 0.00 | C |
| ATOM | 2218 | OG | SER | A 296 | 21.872 | 69.065 | 24.630 | 1.00 | 0.00 | O |
| ATOM | 2219 | N | CYS | A 297 | 21.483 | 65.602 | 24.661 | 1.00 | 0.00 | N |
| ATOM | 2220 | CA | CYS | A 297 | 20.739 | 64.719 | 23.775 | 1.00 | 0.00 | C |
| ATOM | 2221 | C | CYS | A 297 | 19.468 | 65.391 | 23.266 | 1.00 | 0.00 | C |
| ATOM | 2222 | O | CYS | A 297 | 18.582 | 65.737 | 24.045 | 1.00 | 0.00 | O |
| ATOM | 2223 | CB | CYS | A 297 | 20.391 | 63.420 | 24.506 | 1.00 | 0.00 | C |
| ATOM | 2224 | SG | CYS | A 297 | 21.829 | 62.331 | 24.719 | 1.00 | 0.00 | S |
| ATOM | 2225 | N | PRO | A 298 | 19.364 | 65.582 | 21.943 | 1.00 | 0.00 | N |
| ATOM | 2226 | CA | PRO | A 298 | 18.188 | 66.220 | 21.347 | 1.00 | 0.00 | C |
| ATOM | 2227 | C | PRO | A 298 | 16.889 | 65.440 | 21.539 | 1.00 | 0.00 | C |
| ATOM | 2228 | O | PRO | A 298 | 15.801 | 66.000 | 21.407 | 1.00 | 0.00 | O |
| ATOM | 2229 | CB | PRO | A 298 | 18.578 | 66.356 | 19.876 | 1.00 | 0.00 | C |
| ATOM | 2230 | CG | PRO | A 298 | 19.481 | 65.186 | 19.659 | 1.00 | 0.00 | C |
| ATOM | 2231 | CD | PRO | A 298 | 20.337 | 65.201 | 20.903 | 1.00 | 0.00 | C |
| ATOM | 2232 | N | TRP | A 299 | 17.005 | 64.152 | 21.853 | 1.00 | 0.00 | N |
| ATOM | 2233 | CA | TRP | A 299 | 15.828 | 63.313 | 22.065 | 1.00 | 0.00 | C |
| ATOM | 2234 | C | TRP | A 299 | 15.265 | 63.491 | 23.480 | 1.00 | 0.00 | C |
| ATOM | 2235 | O | TRP | A 299 | 14.368 | 62.761 | 23.903 | 1.00 | 0.00 | O |
| ATOM | 2236 | CB | TRP | A 299 | 16.174 | 61.842 | 21.775 | 1.00 | 0.00 | C |
| ATOM | 2237 | CG | TRP | A 299 | 16.534 | 61.619 | 20.324 | 1.00 | 0.00 | C |
| ATOM | 2238 | CD1 | TRP | A 299 | 15.666 | 61.438 | 19.282 | 1.00 | 0.00 | C |
| ATOM | 2239 | CD2 | TRP | A 299 | 17.850 | 61.656 | 19.745 | 1.00 | 0.00 | C |
| ATOM | 2240 | NE1 | TRP | A 299 | 16.357 | 61.367 | 18.093 | 1.00 | 0.00 | N |
| ATOM | 2241 | CE2 | TRP | A 299 | 17.697 | 61.499 | 18.348 | 1.00 | 0.00 | C |
| ATOM | 2242 | CE3 | TRP | A 299 | 19.140 | 61.812 | 20.270 | 1.00 | 0.00 | C |
| ATOM | 2243 | CZ2 | TRP | A 299 | 18.789 | 61.494 | 17.469 | 1.00 | 0.00 | C |
| ATOM | 2244 | CZ3 | TRP | A 299 | 20.226 | 61.806 | 19.396 | 1.00 | 0.00 | C |
| ATOM | 2245 | CH2 | TRP | A 299 | 20.041 | 61.649 | 18.009 | 1.00 | 0.00 | C |
| ATOM | 2246 | N | LYS | A 300 | 15.815 | 64.468 | 24.202 | 1.00 | 0.00 | N |
| ATOM | 2247 | CA | LYS | A 300 | 15.368 | 64.831 | 25.553 | 1.00 | 0.00 | C |
| ATOM | 2248 | C | LYS | A 300 | 15.689 | 63.912 | 26.730 | 1.00 | 0.00 | C |
| ATOM | 2249 | O | LYS | A 300 | 15.274 | 64.190 | 27.853 | 1.00 | 0.00 | O |
| ATOM | 2250 | CB | LYS | A 300 | 13.861 | 65.089 | 25.539 | 1.00 | 0.00 | C |
| ATOM | 2251 | CG | LYS | A 300 | 13.424 | 66.125 | 24.526 | 1.00 | 0.00 | C |
| ATOM | 2252 | CD | LYS | A 300 | 11.918 | 66.321 | 24.569 | 1.00 | 0.00 | C |
| ATOM | 2253 | CE | LYS | A 300 | 11.460 | 67.272 | 23.479 | 1.00 | 0.00 | C |
| ATOM | 2254 | NZ | LYS | A 300 | 12.177 | 68.571 | 23.564 | 1.00 | 0.00 | N |
| ATOM | 2255 | N | VAL | A 301 | 16.403 | 62.820 | 26.490 | 1.00 | 0.00 | N |
| ATOM | 2256 | CA | VAL | A 301 | 16.765 | 61.917 | 27.575 | 1.00 | 0.00 | C |
| ATOM | 2257 | C | VAL | A 301 | 18.287 | 61.901 | 27.673 | 1.00 | 0.00 | C |
| ATOM | 2258 | O | VAL | A 301 | 18.971 | 61.383 | 26.790 | 1.00 | 0.00 | O |
| ATOM | 2259 | CB | VAL | A 301 | 16.247 | 60.485 | 27.325 | 1.00 | 0.00 | C |
| ATOM | 2260 | CG1 | VAL | A 301 | 16.525 | 59.618 | 28.538 | 1.00 | 0.00 | C |
| ATOM | 2261 | CG2 | VAL | A 301 | 14.754 | 60.515 | 27.028 | 1.00 | 0.00 | C |
| ATOM | 2262 | N | PRO | A 302 | 18.836 | 62.477 | 28.752 | 1.00 | 0.00 | N |
| ATOM | 2263 | CA | PRO | A 302 | 20.281 | 62.546 | 28.976 | 1.00 | 0.00 | C |
| ATOM | 2264 | C | PRO | A 302 | 20.907 | 61.253 | 29.467 | 1.00 | 0.00 | C |
| ATOM | 2265 | O | PRO | A 302 | 20.228 | 60.389 | 30.019 | 1.00 | 0.00 | O |
| ATOM | 2266 | CB | PRO | A 302 | 20.403 | 63.653 | 30.010 | 1.00 | 0.00 | C |
| ATOM | 2267 | CG | PRO | A 302 | 19.204 | 63.392 | 30.872 | 1.00 | 0.00 | C |
| ATOM | 2268 | CD | PRO | A 302 | 18.109 | 63.148 | 29.848 | 1.00 | 0.00 | C |
| ATOM | 2269 | N | PRO | A 303 | 22.221 | 61.095 | 29.255 | 1.00 | 0.00 | N |
| ATOM | 2270 | CA | PRO | A 303 | 22.857 | 59.866 | 29.729 | 1.00 | 0.00 | C |
| ATOM | 2271 | C | PRO | A 303 | 22.971 | 59.963 | 31.247 | 1.00 | 0.00 | C |
| ATOM | 2272 | O | PRO | A 303 | 23.018 | 61.064 | 31.803 | 1.00 | 0.00 | O |
| ATOM | 2273 | CB | PRO | A 303 | 24.216 | 59.887 | 29.032 | 1.00 | 0.00 | C |
| ATOM | 2274 | CG | PRO | A 303 | 24.506 | 61.350 | 28.897 | 1.00 | 0.00 | C |
| ATOM | 2275 | CD | PRO | A 303 | 23.167 | 61.932 | 28.494 | 1.00 | 0.00 | C |
| ATOM | 2276 | N | ARG | A 304 | 22.993 | 58.819 | 31.918 | 1.00 | 0.00 | N |
| ATOM | 2277 | CA | ARG | A 304 | 23.111 | 58.800 | 33.369 | 1.00 | 0.00 | C |
| ATOM | 2278 | C | ARG | A 304 | 24.302 | 57.959 | 33.783 | 1.00 | 0.00 | C |
| ATOM | 2279 | O | ARG | A 304 | 24.507 | 56.862 | 33.266 | 1.00 | 0.00 | O |
| ATOM | 2280 | CB | ARG | A 304 | 21.829 | 58.248 | 34.004 | 1.00 | 0.00 | C |
| ATOM | 2281 | CG | ARG | A 304 | 20.698 | 59.268 | 34.084 | 1.00 | 0.00 | C |
| ATOM | 2282 | CD | ARG | A 304 | 19.388 | 58.628 | 34.528 | 1.00 | 0.00 | C |
| ATOM | 2283 | NE | ARG | A 304 | 18.807 | 57.784 | 33.486 | 1.00 | 0.00 | N |
| ATOM | 2284 | CZ | ARG | A 304 | 18.401 | 58.230 | 32.299 | 1.00 | 0.00 | C |
| ATOM | 2285 | NH1 | ARG | A 304 | 18.508 | 59.518 | 31.993 | 1.00 | 0.00 | N |
| ATOM | 2286 | NH2 | ARG | A 304 | 17.884 | 57.389 | 31.413 | 1.00 | 0.00 | N |
| ATOM | 2287 | N | THR | A 305 | 25.093 | 58.485 | 34.712 | 1.00 | 0.00 | N |
| ATOM | 2288 | CA | THR | A 305 | 26.268 | 57.780 | 35.198 | 1.00 | 0.00 | C |
| ATOM | 2289 | C | THR | A 305 | 25.862 | 56.384 | 35.646 | 1.00 | 0.00 | C |
| ATOM | 2290 | O | THR | A 305 | 24.863 | 56.219 | 36.348 | 1.00 | 0.00 | O |
| ATOM | 2291 | CB | THR | A 305 | 26.906 | 58.530 | 36.380 | 1.00 | 0.00 | C |
| ATOM | 2292 | OG1 | THR | A 305 | 27.329 | 59.828 | 35.942 | 1.00 | 0.00 | O |
| ATOM | 2293 | CG2 | THR | A 305 | 28.099 | 57.766 | 36.924 | 1.00 | 0.00 | C |
| ATOM | 2294 | N | ILE | A 306 | 26.628 | 55.380 | 35.231 | 1.00 | 0.00 | N |
| ATOM | 2295 | CA | ILE | A 306 | 26.331 | 54.001 | 35.599 | 1.00 | 0.00 | C |
| ATOM | 2296 | C | ILE | A 306 | 26.739 | 53.742 | 37.046 | 1.00 | 0.00 | C |
| ATOM | 2297 | O | ILE | A 306 | 27.808 | 54.168 | 37.487 | 1.00 | 0.00 | O |
| ATOM | 2298 | CB | ILE | A 306 | 27.079 | 52.997 | 34.694 | 1.00 | 0.00 | C |
| ATOM | 2299 | CG1 | ILE | A 306 | 26.813 | 53.315 | 33.219 | 1.00 | 0.00 | C |
| ATOM | 2300 | CG2 | ILE | A 306 | 26.639 | 51.569 | 35.023 | 1.00 | 0.00 | C |
| ATOM | 2301 | CD1 | ILE | A 306 | 25.349 | 53.255 | 32.815 | 1.00 | 0.00 | C |
| ATOM | 2302 | N | SER | A 307 | 25.876 | 53.045 | 37.780 | 1.00 | 0.00 | N |
| ATOM | 2303 | CA | SER | A 307 | 26.134 | 52.713 | 39.178 | 1.00 | 0.00 | C |
| ATOM | 2304 | C | SER | A 307 | 25.659 | 51.290 | 39.441 | 1.00 | 0.00 | C |
| ATOM | 2305 | O | SER | A 307 | 24.931 | 50.720 | 38.634 | 1.00 | 0.00 | O |
| ATOM | 2306 | CB | SER | A 307 | 25.379 | 53.673 | 40.097 | 1.00 | 0.00 | C |
| ATOM | 2307 | OG | SER | A 307 | 23.980 | 53.548 | 39.909 | 1.00 | 0.00 | O |
| ATOM | 2308 | N | ASP | A 308 | 26.073 | 50.718 | 40.568 | 1.00 | 0.00 | N |
| ATOM | 2309 | CA | ASP | A 308 | 25.661 | 49.361 | 40.923 | 1.00 | 0.00 | C |
| ATOM | 2310 | C | ASP | A 308 | 24.145 | 49.323 | 41.039 | 1.00 | 0.00 | C |
| ATOM | 2311 | O | ASP | A 308 | 23.515 | 48.283 | 40.851 | 1.00 | 0.00 | O |
| ATOM | 2312 | CB | ASP | A 308 | 26.287 | 48.953 | 42.259 | 1.00 | 0.00 | C |
| ATOM | 2313 | CG | ASP | A 308 | 27.775 | 48.703 | 42.152 | 1.00 | 0.00 | C |
| ATOM | 2314 | OD1 | ASP | A 308 | 28.380 | 49.134 | 41.150 | 1.00 | 0.00 | O |
| ATOM | 2315 | OD2 | ASP | A 308 | 28.345 | 48.083 | 43.076 | 1.00 | 0.00 | O |
| ATOM | 2316 | N | GLN | A 309 | 23.574 | 50.483 | 41.336 | 1.00 | 0.00 | N |
| ATOM | 2317 | CA | GLN | A 309 | 22.140 | 50.646 | 41.508 | 1.00 | 0.00 | C |
| ATOM | 2318 | C | GLN | A 309 | 21.326 | 50.648 | 40.213 | 1.00 | 0.00 | C |
| ATOM | 2319 | O | GLN | A 309 | 20.203 | 50.144 | 40.191 | 1.00 | 0.00 | O |
| ATOM | 2320 | CB | GLN | A 309 | 21.891 | 51.932 | 42.309 | 1.00 | 0.00 | C |
| ATOM | 2321 | CG | GLN | A 309 | 20.521 | 52.557 | 42.168 | 1.00 | 0.00 | C |
| ATOM | 2322 | CD | GLN | A 309 | 20.372 | 53.806 | 43.027 | 1.00 | 0.00 | C |
| ATOM | 2323 | OE1 | GLN | A 309 | 19.547 | 54.680 | 42.743 | 1.00 | 0.00 | O |
| ATOM | 2324 | NE2 | GLN | A 309 | 21.166 | 53.890 | 44.089 | 1.00 | 0.00 | N |
| ATOM | 2325 | N | ASN | A 310 | 21.879 | 51.195 | 39.133 | 1.00 | 0.00 | N |
| ATOM | 2326 | CA | ASN | A 310 | 21.144 | 51.239 | 37.871 | 1.00 | 0.00 | C |
| ATOM | 2327 | C | ASN | A 310 | 21.748 | 50.415 | 36.736 | 1.00 | 0.00 | C |
| ATOM | 2328 | O | ASN | A 310 | 21.164 | 50.335 | 35.660 | 1.00 | 0.00 | O |
| ATOM | 2329 | CB | ASN | A 310 | 20.994 | 52.687 | 37.387 | 1.00 | 0.00 | C |
| ATOM | 2330 | CG | ASN | A 310 | 22.328 | 53.335 | 37.044 | 1.00 | 0.00 | C |
| ATOM | 2331 | OD1 | ASN | A 310 | 23.268 | 52.663 | 36.620 | 1.00 | 0.00 | O |
| ATOM | 2332 | ND2 | ASN | A 310 | 22.408 | 54.650 | 37.211 | 1.00 | 0.00 | N |
| ATOM | 2333 | N | VAL | A 311 | 22.906 | 49.806 | 36.972 | 1.00 | 0.00 | N |
| ATOM | 2334 | CA | VAL | A 311 | 23.582 | 49.028 | 35.931 | 1.00 | 0.00 | C |
| ATOM | 2335 | C | VAL | A 311 | 22.748 | 47.908 | 35.302 | 1.00 | 0.00 | C |
| ATOM | 2336 | O | VAL | A 311 | 22.863 | 47.649 | 34.104 | 1.00 | 0.00 | O |
| ATOM | 2337 | CB | VAL | A 311 | 24.913 | 48.433 | 36.453 | 1.00 | 0.00 | C |
| ATOM | 2338 | CG1 | VAL | A 311 | 24.639 | 47.316 | 37.448 | 1.00 | 0.00 | C |
| ATOM | 2339 | CG2 | VAL | A 311 | 25.756 | 47.933 | 35.278 | 1.00 | 0.00 | C |
| ATOM | 2340 | N | ALA | A 312 | 21.910 | 47.244 | 36.095 | 1.00 | 0.00 | N |
| ATOM | 2341 | CA | ALA | A 312 | 21.083 | 46.167 | 35.557 | 1.00 | 0.00 | C |
| ATOM | 2342 | C | ALA | A 312 | 20.067 | 46.703 | 34.550 | 1.00 | 0.00 | C |
| ATOM | 2343 | O | ALA | A 312 | 19.868 | 46.114 | 33.486 | 1.00 | 0.00 | O |
| ATOM | 2344 | CB | ALA | A 312 | 20.363 | 45.429 | 36.693 | 1.00 | 0.00 | C |
| ATOM | 2345 | N | ALA | A 313 | 19.431 | 47.823 | 34.887 | 1.00 | 0.00 | N |
| ATOM | 2346 | CA | ALA | A 313 | 18.433 | 48.439 | 34.015 | 1.00 | 0.00 | C |
| ATOM | 2347 | C | ALA | A 313 | 19.079 | 49.081 | 32.790 | 1.00 | 0.00 | C |
| ATOM | 2348 | O | ALA | A 313 | 18.541 | 49.017 | 31.682 | 1.00 | 0.00 | O |
| ATOM | 2349 | CB | ALA | A 313 | 17.641 | 49.484 | 34.787 | 1.00 | 0.00 | C |
| ATOM | 2350 | N | ARG | A 314 | 20.226 | 49.716 | 32.996 | 1.00 | 0.00 | N |
| ATOM | 2351 | CA | ARG | A 314 | 20.941 | 50.358 | 31.896 | 1.00 | 0.00 | C |
| ATOM | 2352 | C | ARG | A 314 | 21.411 | 49.291 | 30.907 | 1.00 | 0.00 | C |
| ATOM | 2353 | O | ARG | A 314 | 21.307 | 49.464 | 29.691 | 1.00 | 0.00 | O |
| ATOM | 2354 | CB | ARG | A 314 | 22.147 | 51.130 | 32.435 | 1.00 | 0.00 | C |
| ATOM | 2355 | CG | ARG | A 314 | 21.805 | 52.288 | 33.365 | 1.00 | 0.00 | C |
| ATOM | 2356 | CD | ARG | A 314 | 21.451 | 53.557 | 32.599 | 1.00 | 0.00 | C |
| ATOM | 2357 | NE | ARG | A 314 | 20.085 | 53.557 | 32.090 | 1.00 | 0.00 | N |
| ATOM | 2358 | CZ | ARG | A 314 | 19.594 | 54.486 | 31.276 | 1.00 | 0.00 | C |
| ATOM | 2359 | NH1 | ARG | A 314 | 20.361 | 55.489 | 30.871 | 1.00 | 0.00 | N |
| ATOM | 2360 | NH2 | ARG | A 314 | 18.334 | 54.422 | 30.875 | 1.00 | 0.00 | N |
| ATOM | 2361 | N | SER | A 315 | 21.919 | 48.184 | 31.439 | 1.00 | 0.00 | N |
| ATOM | 2362 | CA | SER | A 315 | 22.404 | 47.081 | 30.611 | 1.00 | 0.00 | C |
| ATOM | 2363 | C | SER | A 315 | 21.270 | 46.457 | 29.815 | 1.00 | 0.00 | C |
| ATOM | 2364 | O | SER | A 315 | 21.425 | 46.141 | 28.636 | 1.00 | 0.00 | O |
| ATOM | 2365 | CB | SER | A 315 | 23.063 | 46.008 | 31.483 | 1.00 | 0.00 | C |
| ATOM | 2366 | OG | SER | A 315 | 24.249 | 46.498 | 32.083 | 1.00 | 0.00 | O |
| ATOM | 2367 | N | ASP | A 316 | 20.121 | 46.283 | 30.460 | 1.00 | 0.00 | N |
| ATOM | 2368 | CA | ASP | A 316 | 18.974 | 45.692 | 29.788 | 1.00 | 0.00 | C |
| ATOM | 2369 | C | ASP | A 316 | 18.605 | 46.540 | 28.573 | 1.00 | 0.00 | C |
| ATOM | 2370 | O | ASP | A 316 | 18.311 | 46.009 | 27.501 | 1.00 | 0.00 | O |
| ATOM | 2371 | CB | ASP | A 316 | 17.793 | 45.593 | 30.758 | 1.00 | 0.00 | C |
| ATOM | 2372 | CG | ASP | A 316 | 16.715 | 44.647 | 30.268 | 1.00 | 0.00 | C |
| ATOM | 2373 | OD1 | ASP | A 316 | 17.040 | 43.482 | 29.946 | 1.00 | 0.00 | O |
| ATOM | 2374 | OD2 | ASP | A 316 | 15.542 | 45.068 | 30.211 | 1.00 | 0.00 | O |
| ATOM | 2375 | N | LEU | A 317 | 18.633 | 47.861 | 28.738 | 1.00 | 0.00 | N |
| ATOM | 2376 | CA | LEU | A 317 | 18.318 | 48.766 | 27.638 | 1.00 | 0.00 | C |
| ATOM | 2377 | C | LEU | A 317 | 19.362 | 48.680 | 26.531 | 1.00 | 0.00 | C |
| ATOM | 2378 | O | LEU | A 317 | 19.024 | 48.567 | 25.353 | 1.00 | 0.00 | O |
| ATOM | 2379 | CB | LEU | A 317 | 18.246 | 50.214 | 28.133 | 1.00 | 0.00 | C |
| ATOM | 2380 | CG | LEU | A 317 | 16.879 | 50.764 | 28.537 | 1.00 | 0.00 | C |
| ATOM | 2381 | CD1 | LEU | A 317 | 17.049 | 52.176 | 29.092 | 1.00 | 0.00 | C |
| ATOM | 2382 | CD2 | LEU | A 317 | 15.952 | 50.777 | 27.330 | 1.00 | 0.00 | C |
| ATOM | 2383 | N | LEU | A 318 | 20.632 | 48.738 | 26.918 | 1.00 | 0.00 | N |
| ATOM | 2384 | CA | LEU | A 318 | 21.725 | 48.693 | 25.952 | 1.00 | 0.00 | C |
| ATOM | 2385 | C | LEU | A 318 | 21.791 | 47.364 | 25.212 | 1.00 | 0.00 | C |
| ATOM | 2386 | O | LEU | A 318 | 21.826 | 47.329 | 23.981 | 1.00 | 0.00 | O |
| ATOM | 2387 | CB | LEU | A 318 | 23.061 | 48.957 | 26.653 | 1.00 | 0.00 | C |
| ATOM | 2388 | CG | LEU | A 318 | 24.279 | 49.102 | 25.734 | 1.00 | 0.00 | C |
| ATOM | 2389 | CD1 | LEU | A 318 | 24.038 | 50.236 | 24.743 | 1.00 | 0.00 | C |
| ATOM | 2390 | CD2 | LEU | A 318 | 25.528 | 49.371 | 26.569 | 1.00 | 0.00 | C |
| ATOM | 2391 | N | VAL | A 319 | 21.815 | 46.268 | 25.962 | 1.00 | 0.00 | N |
| ATOM | 2392 | CA | VAL | A 319 | 21.879 | 44.950 | 25.349 | 1.00 | 0.00 | C |
| ATOM | 2393 | C | VAL | A 319 | 20.740 | 44.769 | 24.351 | 1.00 | 0.00 | C |
| ATOM | 2394 | O | VAL | A 319 | 20.922 | 44.165 | 23.297 | 1.00 | 0.00 | O |
| ATOM | 2395 | CB | VAL | A 319 | 21.820 | 43.839 | 26.417 | 1.00 | 0.00 | C |
| ATOM | 2396 | CG1 | VAL | A 319 | 21.629 | 42.483 | 25.755 | 1.00 | 0.00 | C |
| ATOM | 2397 | CG2 | VAL | A 319 | 23.107 | 43.842 | 27.230 | 1.00 | 0.00 | C |
| ATOM | 2398 | N | ASP | A 320 | 19.567 | 45.304 | 24.675 | 1.00 | 0.00 | N |
| ATOM | 2399 | CA | ASP | A 320 | 18.420 | 45.187 | 23.779 | 1.00 | 0.00 | C |
| ATOM | 2400 | C | ASP | A 320 | 18.716 | 45.878 | 22.446 | 1.00 | 0.00 | C |
| ATOM | 2401 | O | ASP | A 320 | 18.350 | 45.378 | 21.382 | 1.00 | 0.00 | O |
| ATOM | 2402 | CB | ASP | A 320 | 17.179 | 45.794 | 24.435 | 1.00 | 0.00 | C |
| ATOM | 2403 | CG | ASP | A 320 | 15.976 | 45.789 | 23.522 | 1.00 | 0.00 | C |
| ATOM | 2404 | OD1 | ASP | A 320 | 15.527 | 46.886 | 23.133 | 1.00 | 0.00 | O |
| ATOM | 2405 | OD2 | ASP | A 320 | 15.481 | 44.692 | 23.191 | 1.00 | 0.00 | O |
| ATOM | 2406 | N | GLN | A 321 | 19.376 | 47.031 | 22.509 | 1.00 | 0.00 | N |
| ATOM | 2407 | CA | GLN | A 321 | 19.741 | 47.761 | 21.298 | 1.00 | 0.00 | C |
| ATOM | 2408 | C | GLN | A 321 | 20.740 | 46.935 | 20.501 | 1.00 | 0.00 | C |
| ATOM | 2409 | O | GLN | A 321 | 20.630 | 46.816 | 19.279 | 1.00 | 0.00 | O |
| ATOM | 2410 | CB | GLN | A 321 | 20.371 | 49.109 | 21.654 | 1.00 | 0.00 | C |
| ATOM | 2411 | CG | GLN | A 321 | 19.366 | 50.146 | 22.102 | 1.00 | 0.00 | C |
| ATOM | 2412 | CD | GLN | A 321 | 18.323 | 50.413 | 21.040 | 1.00 | 0.00 | C |
| ATOM | 2413 | OE1 | GLN | A 321 | 18.645 | 50.834 | 19.926 | 1.00 | 0.00 | O |
| ATOM | 2414 | NE2 | GLN | A 321 | 17.062 | 50.164 | 21.375 | 1.00 | 0.00 | N |
| ATOM | 2415 | N | TRP | A 322 | 21.718 | 46.368 | 21.203 | 1.00 | 0.00 | N |
| ATOM | 2416 | CA | TRP | A 322 | 22.738 | 45.544 | 20.568 | 1.00 | 0.00 | C |
| ATOM | 2417 | C | TRP | A 322 | 22.133 | 44.351 | 19.837 | 1.00 | 0.00 | C |
| ATOM | 2418 | O | TRP | A 322 | 22.476 | 44.081 | 18.685 | 1.00 | 0.00 | O |
| ATOM | 2419 | CB | TRP | A 322 | 23.744 | 45.027 | 21.600 | 1.00 | 0.00 | C |
| ATOM | 2420 | CG | TRP | A 322 | 24.688 | 46.060 | 22.143 | 1.00 | 0.00 | C |
| ATOM | 2421 | CD1 | TRP | A 322 | 24.907 | 47.315 | 21.656 | 1.00 | 0.00 | C |
| ATOM | 2422 | CD2 | TRP | A 322 | 25.583 | 45.898 | 23.249 | 1.00 | 0.00 | C |
| ATOM | 2423 | NE1 | TRP | A 322 | 25.887 | 47.945 | 22.390 | 1.00 | 0.00 | N |
| ATOM | 2424 | CE2 | TRP | A 322 | 26.319 | 47.097 | 23.373 | 1.00 | 0.00 | C |
| ATOM | 2425 | CE3 | TRP | A 322 | 25.837 | 44.853 | 24.148 | 1.00 | 0.00 | C |
| ATOM | 2426 | CZ2 | TRP | A 322 | 27.292 | 47.281 | 24.363 | 1.00 | 0.00 | C |
| ATOM | 2427 | CZ3 | TRP | A 322 | 26.805 | 45.034 | 25.132 | 1.00 | 0.00 | C |
| ATOM | 2428 | CH2 | TRP | A 322 | 27.520 | 46.240 | 25.230 | 1.00 | 0.00 | C |
| ATOM | 2429 | N | LYS | A 323 | 21.246 | 43.627 | 20.512 | 1.00 | 0.00 | N |
| ATOM | 2430 | CA | LYS | A 323 | 20.621 | 42.461 | 19.899 | 1.00 | 0.00 | C |
| ATOM | 2431 | C | LYS | A 323 | 19.759 | 42.830 | 18.699 | 1.00 | 0.00 | C |
| ATOM | 2432 | O | LYS | A 323 | 19.625 | 42.046 | 17.763 | 1.00 | 0.00 | O |
| ATOM | 2433 | CB | LYS | A 323 | 19.817 | 41.680 | 20.943 | 1.00 | 0.00 | C |
| ATOM | 2434 | CG | LYS | A 323 | 20.730 | 40.891 | 21.882 | 1.00 | 0.00 | C |
| ATOM | 2435 | CD | LYS | A 323 | 19.956 | 40.047 | 22.883 | 1.00 | 0.00 | C |
| ATOM | 2436 | CE | LYS | A 323 | 20.914 | 39.198 | 23.710 | 1.00 | 0.00 | C |
| ATOM | 2437 | NZ | LYS | A 323 | 20.199 | 38.349 | 24.700 | 1.00 | 0.00 | N |
| ATOM | 2438 | N | LYS | A 324 | 19.181 | 44.024 | 18.716 | 1.00 | 0.00 | N |
| ATOM | 2439 | CA | LYS | A 324 | 18.376 | 44.457 | 17.581 | 1.00 | 0.00 | C |
| ATOM | 2440 | C | LYS | A 324 | 19.313 | 44.754 | 16.415 | 1.00 | 0.00 | C |
| ATOM | 2441 | O | LYS | A 324 | 19.041 | 44.373 | 15.276 | 1.00 | 0.00 | O |
| ATOM | 2442 | CB | LYS | A 324 | 17.551 | 45.693 | 17.945 | 1.00 | 0.00 | C |
| ATOM | 2443 | CG | LYS | A 324 | 16.374 | 45.365 | 18.855 | 1.00 | 0.00 | C |
| ATOM | 2444 | CD | LYS | A 324 | 15.586 | 46.600 | 19.267 | 1.00 | 0.00 | C |
| ATOM | 2445 | CE | LYS | A 324 | 14.390 | 46.201 | 20.135 | 1.00 | 0.00 | C |
| ATOM | 2446 | NZ | LYS | A 324 | 13.676 | 47.386 | 20.693 | 1.00 | 0.00 | N |
| ATOM | 2447 | N | LYS | A 325 | 20.430 | 45.417 | 16.700 | 1.00 | 0.00 | N |
| ATOM | 2448 | CA | LYS | A 325 | 21.392 | 45.728 | 15.646 | 1.00 | 0.00 | C |
| ATOM | 2449 | C | LYS | A 325 | 21.941 | 44.418 | 15.076 | 1.00 | 0.00 | C |
| ATOM | 2450 | O | LYS | A 325 | 22.101 | 44.276 | 13.863 | 1.00 | 0.00 | O |
| ATOM | 2451 | CB | LYS | A 325 | 22.545 | 46.576 | 16.200 | 1.00 | 0.00 | C |
| ATOM | 2452 | CG | LYS | A 325 | 23.439 | 47.172 | 15.117 | 1.00 | 0.00 | C |
| ATOM | 2453 | CD | LYS | A 325 | 24.583 | 47.999 | 15.701 | 1.00 | 0.00 | C |
| ATOM | 2454 | CE | LYS | A 325 | 25.399 | 48.653 | 14.588 | 1.00 | 0.00 | C |
| ATOM | 2455 | NZ | LYS | A 325 | 26.594 | 49.372 | 15.107 | 1.00 | 0.00 | N |
| ATOM | 2456 | N | ALA | A 326 | 22.212 | 43.461 | 15.961 | 1.00 | 0.00 | N |
| ATOM | 2457 | CA | ALA | A 326 | 22.746 | 42.157 | 15.566 | 1.00 | 0.00 | C |
| ATOM | 2458 | C | ALA | A 326 | 21.847 | 41.398 | 14.587 | 1.00 | 0.00 | C |
| ATOM | 2459 | O | ALA | A 326 | 22.321 | 40.548 | 13.831 | 1.00 | 0.00 | O |
| ATOM | 2460 | CB | ALA | A 326 | 22.993 | 41.302 | 16.805 | 1.00 | 0.00 | C |
| ATOM | 2461 | N | GLU | A 327 | 20.554 | 41.697 | 14.604 | 1.00 | 0.00 | N |
| ATOM | 2462 | CA | GLU | A 327 | 19.610 | 41.031 | 13.708 | 1.00 | 0.00 | C |
| ATOM | 2463 | C | GLU | A 327 | 19.823 | 41.409 | 12.248 | 1.00 | 0.00 | C |
| ATOM | 2464 | O | GLU | A 327 | 19.373 | 40.705 | 11.345 | 1.00 | 0.00 | O |
| ATOM | 2465 | CB | GLU | A 327 | 18.173 | 41.375 | 14.099 | 1.00 | 0.00 | C |
| ATOM | 2466 | CG | GLU | A 327 | 17.644 | 40.575 | 15.262 | 1.00 | 0.00 | C |
| ATOM | 2467 | CD | GLU | A 327 | 17.587 | 39.091 | 14.952 | 1.00 | 0.00 | C |
| ATOM | 2468 | OE1 | GLU | A 327 | 16.900 | 38.710 | 13.981 | 1.00 | 0.00 | O |
| ATOM | 2469 | OE2 | GLU | A 327 | 18.231 | 38.310 | 15.676 | 1.00 | 0.00 | O |
| ATOM | 2470 | N | LEU | A 328 | 20.510 | 42.523 | 12.020 | 1.00 | 0.00 | N |
| ATOM | 2471 | CA | LEU | A 328 | 20.750 | 42.989 | 10.663 | 1.00 | 0.00 | C |
| ATOM | 2472 | C | LEU | A 328 | 21.969 | 42.334 | 10.022 | 1.00 | 0.00 | C |
| ATOM | 2473 | O | LEU | A 328 | 22.225 | 42.539 | 8.836 | 1.00 | 0.00 | O |
| ATOM | 2474 | CB | LEU | A 328 | 20.910 | 44.515 | 10.654 | 1.00 | 0.00 | C |
| ATOM | 2475 | CG | LEU | A 328 | 19.804 | 45.321 | 11.345 | 1.00 | 0.00 | C |
| ATOM | 2476 | CD1 | LEU | A 328 | 20.034 | 46.810 | 11.114 | 1.00 | 0.00 | C |
| ATOM | 2477 | CD2 | LEU | A 328 | 18.436 | 44.902 | 10.813 | 1.00 | 0.00 | C |
| ATOM | 2478 | N | TYR | A 329 | 22.713 | 41.549 | 10.800 | 1.00 | 0.00 | N |
| ATOM | 2479 | CA | TYR | A 329 | 23.906 | 40.872 | 10.291 | 1.00 | 0.00 | C |
| ATOM | 2480 | C | TYR | A 329 | 23.843 | 39.349 | 10.449 | 1.00 | 0.00 | C |
| ATOM | 2481 | O | TYR | A 329 | 22.952 | 38.823 | 11.121 | 1.00 | 0.00 | O |
| ATOM | 2482 | CB | TYR | A 329 | 25.163 | 41.451 | 10.956 | 1.00 | 0.00 | C |
| ATOM | 2483 | CG | TYR | A 329 | 25.356 | 42.922 | 10.639 | 1.00 | 0.00 | C |
| ATOM | 2484 | CD1 | TYR | A 329 | 24.736 | 43.912 | 11.407 | 1.00 | 0.00 | C |
| ATOM | 2485 | CD2 | TYR | A 329 | 26.106 | 43.323 | 9.530 | 1.00 | 0.00 | C |
| ATOM | 2486 | CE1 | TYR | A 329 | 24.857 | 45.263 | 11.077 | 1.00 | 0.00 | C |
| ATOM | 2487 | CE2 | TYR | A 329 | 26.231 | 44.670 | 9.192 | 1.00 | 0.00 | C |
| ATOM | 2488 | CZ | TYR | A 329 | 25.604 | 45.634 | 9.969 | 1.00 | 0.00 | C |
| ATOM | 2489 | OH | TYR | A 329 | 25.717 | 46.965 | 9.634 | 1.00 | 0.00 | O |
| ATOM | 2490 | N | ARG | A 330 | 24.795 | 38.648 | 9.837 | 1.00 | 0.00 | N |
| ATOM | 2491 | CA | ARG | A 330 | 24.799 | 37.188 | 9.845 | 1.00 | 0.00 | C |
| ATOM | 2492 | C | ARG | A 330 | 25.619 | 36.414 | 10.878 | 1.00 | 0.00 | C |
| ATOM | 2493 | O | ARG | A 330 | 25.375 | 35.225 | 11.076 | 1.00 | 0.00 | O |
| ATOM | 2494 | CB | ARG | A 330 | 25.169 | 36.689 | 8.442 | 1.00 | 0.00 | C |
| ATOM | 2495 | CG | ARG | A 330 | 24.273 | 37.270 | 7.356 | 1.00 | 0.00 | C |
| ATOM | 2496 | CD | ARG | A 330 | 24.492 | 36.623 | 5.994 | 1.00 | 0.00 | C |
| ATOM | 2497 | NE | ARG | A 330 | 23.565 | 37.189 | 5.020 | 1.00 | 0.00 | N |
| ATOM | 2498 | CZ | ARG | A 330 | 23.295 | 36.657 | 3.831 | 1.00 | 0.00 | C |
| ATOM | 2499 | NH1 | ARG | A 330 | 23.885 | 35.530 | 3.450 | 1.00 | 0.00 | N |
| ATOM | 2500 | NH2 | ARG | A 330 | 22.425 | 37.251 | 3.024 | 1.00 | 0.00 | N |
| ATOM | 2501 | N | THR | A 331 | 26.590 | 37.049 | 11.528 | 1.00 | 0.00 | N |
| ATOM | 2502 | CA | THR | A 331 | 27.386 | 36.324 | 12.520 | 1.00 | 0.00 | C |
| ATOM | 2503 | C | THR | A 331 | 27.006 | 36.729 | 13.937 | 1.00 | 0.00 | C |
| ATOM | 2504 | O | THR | A 331 | 26.151 | 37.596 | 14.140 | 1.00 | 0.00 | O |
| ATOM | 2505 | CB | THR | A 331 | 28.902 | 36.579 | 12.352 | 1.00 | 0.00 | C |
| ATOM | 2506 | OG1 | THR | A 331 | 29.218 | 37.896 | 12.815 | 1.00 | 0.00 | O |
| ATOM | 2507 | CG2 | THR | A 331 | 29.318 | 36.440 | 10.887 | 1.00 | 0.00 | C |
| ATOM | 2508 | N | ASN | A 332 | 27.651 | 36.099 | 14.914 | 1.00 | 0.00 | N |
| ATOM | 2509 | CA | ASN | A 332 | 27.398 | 36.403 | 16.316 | 1.00 | 0.00 | C |
| ATOM | 2510 | C | ASN | A 332 | 28.462 | 37.373 | 16.835 | 1.00 | 0.00 | C |
| ATOM | 2511 | O | ASN | A 332 | 28.720 | 37.454 | 18.038 | 1.00 | 0.00 | O |
| ATOM | 2512 | CB | ASN | A 332 | 27.379 | 35.109 | 17.150 | 1.00 | 0.00 | C |
| ATOM | 2513 | CG | ASN | A 332 | 28.746 | 34.448 | 17.254 | 1.00 | 0.00 | C |
| ATOM | 2514 | OD1 | ASN | A 332 | 29.527 | 34.446 | 16.302 | 1.00 | 0.00 | O |
| ATOM | 2515 | ND2 | ASN | A 332 | 29.033 | 33.866 | 18.417 | 1.00 | 0.00 | N |
| ATOM | 2516 | N | VAL | A 333 | 29.079 | 38.106 | 15.909 | 1.00 | 0.00 | N |
| ATOM | 2517 | CA | VAL | A 333 | 30.098 | 39.094 | 16.248 | 1.00 | 0.00 | C |
| ATOM | 2518 | C | VAL | A 333 | 29.524 | 40.454 | 15.850 | 1.00 | 0.00 | C |
| ATOM | 2519 | O | VAL | A 333 | 29.201 | 40.674 | 14.684 | 1.00 | 0.00 | O |
| ATOM | 2520 | CB | VAL | A 333 | 31.413 | 38.843 | 15.472 | 1.00 | 0.00 | C |
| ATOM | 2521 | CG1 | VAL | A 333 | 32.457 | 39.884 | 15.864 | 1.00 | 0.00 | C |
| ATOM | 2522 | CG2 | VAL | A 333 | 31.925 | 37.442 | 15.761 | 1.00 | 0.00 | C |
| ATOM | 2523 | N | LEU | A 334 | 29.406 | 41.361 | 16.817 | 1.00 | 0.00 | N |
| ATOM | 2524 | CA | LEU | A 334 | 28.816 | 42.671 | 16.564 | 1.00 | 0.00 | C |
| ATOM | 2525 | C | LEU | A 334 | 29.759 | 43.866 | 16.719 | 1.00 | 0.00 | C |
| ATOM | 2526 | O | LEU | A 334 | 30.469 | 43.988 | 17.715 | 1.00 | 0.00 | O |
| ATOM | 2527 | CB | LEU | A 334 | 27.609 | 42.859 | 17.488 | 1.00 | 0.00 | C |
| ATOM | 2528 | CG | LEU | A 334 | 26.795 | 44.143 | 17.332 | 1.00 | 0.00 | C |
| ATOM | 2529 | CD1 | LEU | A 334 | 26.072 | 44.128 | 15.988 | 1.00 | 0.00 | C |
| ATOM | 2530 | CD2 | LEU | A 334 | 25.797 | 44.254 | 18.475 | 1.00 | 0.00 | C |
| ATOM | 2531 | N | LEU | A 335 | 29.741 | 44.756 | 15.730 | 1.00 | 0.00 | N |
| ATOM | 2532 | CA | LEU | A 335 | 30.584 | 45.952 | 15.755 | 1.00 | 0.00 | C |
| ATOM | 2533 | C | LEU | A 335 | 29.798 | 47.139 | 16.297 | 1.00 | 0.00 | C |
| ATOM | 2534 | O | LEU | A 335 | 28.721 | 47.455 | 15.796 | 1.00 | 0.00 | O |
| ATOM | 2535 | CB | LEU | A 335 | 31.081 | 46.287 | 14.346 | 1.00 | 0.00 | C |
| ATOM | 2536 | CG | LEU | A 335 | 31.843 | 47.612 | 14.214 | 1.00 | 0.00 | C |
| ATOM | 2537 | CD1 | LEU | A 335 | 33.147 | 47.540 | 14.993 | 1.00 | 0.00 | C |
| ATOM | 2538 | CD2 | LEU | A 335 | 32.113 | 47.903 | 12.747 | 1.00 | 0.00 | C |
| ATOM | 2539 | N | ILE | A 336 | 30.343 | 47.797 | 17.318 | 1.00 | 0.00 | N |
| ATOM | 2540 | CA | ILE | A 336 | 29.697 | 48.959 | 17.922 | 1.00 | 0.00 | C |
| ATOM | 2541 | C | ILE | A 336 | 30.683 | 50.123 | 18.003 | 1.00 | 0.00 | C |
| ATOM | 2542 | O | ILE | A 336 | 31.436 | 50.244 | 18.965 | 1.00 | 0.00 | O |
| ATOM | 2543 | CB | ILE | A 336 | 29.183 | 48.652 | 19.360 | 1.00 | 0.00 | C |
| ATOM | 2544 | CG1 | ILE | A 336 | 28.160 | 47.507 | 19.331 | 1.00 | 0.00 | C |
| ATOM | 2545 | CG2 | ILE | A 336 | 28.537 | 49.895 | 19.965 | 1.00 | 0.00 | C |
| ATOM | 2546 | CD1 | ILE | A 336 | 26.851 | 47.848 | 18.628 | 1.00 | 0.00 | C |
| ATOM | 2547 | N | PRO | A 337 | 30.711 | 50.981 | 16.974 | 1.00 | 0.00 | N |
| ATOM | 2548 | CA | PRO | A 337 | 31.636 | 52.119 | 17.017 | 1.00 | 0.00 | C |
| ATOM | 2549 | C | PRO | A 337 | 31.256 | 53.035 | 18.179 | 1.00 | 0.00 | C |
| ATOM | 2550 | O | PRO | A 337 | 30.080 | 53.150 | 18.515 | 1.00 | 0.00 | O |
| ATOM | 2551 | CB | PRO | A 337 | 31.404 | 52.807 | 15.672 | 1.00 | 0.00 | C |
| ATOM | 2552 | CG | PRO | A 337 | 30.981 | 51.684 | 14.778 | 1.00 | 0.00 | C |
| ATOM | 2553 | CD | PRO | A 337 | 30.044 | 50.892 | 15.665 | 1.00 | 0.00 | C |
| ATOM | 2554 | N | LEU | A 338 | 32.249 | 53.678 | 18.789 | 1.00 | 0.00 | N |
| ATOM | 2555 | CA | LEU | A 338 | 31.994 | 54.601 | 19.893 | 1.00 | 0.00 | C |
| ATOM | 2556 | C | LEU | A 338 | 32.802 | 55.875 | 19.671 | 1.00 | 0.00 | C |
| ATOM | 2557 | O | LEU | A 338 | 33.941 | 55.972 | 20.112 | 1.00 | 0.00 | O |
| ATOM | 2558 | CB | LEU | A 338 | 32.396 | 53.981 | 21.238 | 1.00 | 0.00 | C |
| ATOM | 2559 | CG | LEU | A 338 | 32.108 | 54.865 | 22.461 | 1.00 | 0.00 | C |
| ATOM | 2560 | CD1 | LEU | A 338 | 30.606 | 54.919 | 22.707 | 1.00 | 0.00 | C |
| ATOM | 2561 | CD2 | LEU | A 338 | 32.827 | 54.315 | 23.693 | 1.00 | 0.00 | C |
| ATOM | 2562 | N | GLY | A 339 | 32.211 | 56.849 | 18.983 | 1.00 | 0.00 | N |
| ATOM | 2563 | CA | GLY | A 339 | 32.924 | 58.091 | 18.731 | 1.00 | 0.00 | C |
| ATOM | 2564 | C | GLY | A 339 | 32.131 | 59.114 | 17.943 | 1.00 | 0.00 | C |
| ATOM | 2565 | O | GLY | A 339 | 30.970 | 58.889 | 17.596 | 1.00 | 0.00 | O |
| ATOM | 2566 | N | ASP | A 340 | 32.769 | 60.244 | 17.658 | 1.00 | 0.00 | N |
| ATOM | 2567 | CA | ASP | A 340 | 32.141 | 61.329 | 16.919 | 1.00 | 0.00 | C |
| ATOM | 2568 | C | ASP | A 340 | 33.265 | 62.270 | 16.472 | 1.00 | 0.00 | C |
| ATOM | 2569 | O | ASP | A 340 | 34.445 | 61.949 | 16.626 | 1.00 | 0.00 | O |
| ATOM | 2570 | CB | ASP | A 340 | 31.165 | 62.076 | 17.831 | 1.00 | 0.00 | C |
| ATOM | 2571 | CG | ASP | A 340 | 30.043 | 62.759 | 17.068 | 1.00 | 0.00 | C |
| ATOM | 2572 | OD1 | ASP | A 340 | 30.242 | 63.118 | 15.886 | 1.00 | 0.00 | O |
| ATOM | 2573 | OD2 | ASP | A 340 | 28.961 | 62.952 | 17.664 | 1.00 | 0.00 | O |
| ATOM | 2574 | N | ASP | A 341 | 32.902 | 63.432 | 15.938 | 1.00 | 0.00 | N |
| ATOM | 2575 | CA | ASP | A 341 | 33.896 | 64.396 | 15.468 | 1.00 | 0.00 | C |
| ATOM | 2576 | C | ASP | A 341 | 34.810 | 64.907 | 16.574 | 1.00 | 0.00 | C |
| ATOM | 2577 | O | ASP | A 341 | 34.343 | 65.379 | 17.612 | 1.00 | 0.00 | O |
| ATOM | 2578 | CB | ASP | A 341 | 33.211 | 65.593 | 14.803 | 1.00 | 0.00 | C |
| ATOM | 2579 | CG | ASP | A 341 | 32.537 | 65.230 | 13.494 | 1.00 | 0.00 | C |
| ATOM | 2580 | OD1 | ASP | A 341 | 32.491 | 64.027 | 13.165 | 1.00 | 0.00 | O |
| ATOM | 2581 | OD2 | ASP | A 341 | 32.053 | 66.150 | 12.799 | 1.00 | 0.00 | O |
| ATOM | 2582 | N | PHE | A 342 | 36.113 | 64.813 | 16.330 | 1.00 | 0.00 | N |
| ATOM | 2583 | CA | PHE | A 342 | 37.128 | 65.274 | 17.264 | 1.00 | 0.00 | C |
| ATOM | 2584 | C | PHE | A 342 | 36.835 | 64.915 | 18.717 | 1.00 | 0.00 | C |
| ATOM | 2585 | O | PHE | A 342 | 36.977 | 65.748 | 19.619 | 1.00 | 0.00 | O |
| ATOM | 2586 | CB | PHE | A 342 | 37.314 | 66.788 | 17.125 | 1.00 | 0.00 | C |
| ATOM | 2587 | CG | PHE | A 342 | 37.850 | 67.213 | 15.781 | 1.00 | 0.00 | C |
| ATOM | 2588 | CD1 | PHE | A 342 | 36.986 | 67.554 | 14.743 | 1.00 | 0.00 | C |
| ATOM | 2589 | CD2 | PHE | A 342 | 39.223 | 67.254 | 15.549 | 1.00 | 0.00 | C |
| ATOM | 2590 | CE1 | PHE | A 342 | 37.478 | 67.932 | 13.492 | 1.00 | 0.00 | C |
| ATOM | 2591 | CE2 | PHE | A 342 | 39.728 | 67.628 | 14.303 | 1.00 | 0.00 | C |
| ATOM | 2592 | CZ | PHE | A 342 | 38.853 | 67.969 | 13.272 | 1.00 | 0.00 | C |
| ATOM | 2593 | N | ARG | A 343 | 36.432 | 63.667 | 18.936 | 1.00 | 0.00 | N |
| ATOM | 2594 | CA | ARG | A 343 | 36.138 | 63.187 | 20.278 | 1.00 | 0.00 | C |
| ATOM | 2595 | C | ARG | A 343 | 37.402 | 62.725 | 20.993 | 1.00 | 0.00 | C |
| ATOM | 2596 | O | ARG | A 343 | 38.473 | 62.616 | 20.389 | 1.00 | 0.00 | O |
| ATOM | 2597 | CB | ARG | A 343 | 35.131 | 62.033 | 20.226 | 1.00 | 0.00 | C |
| ATOM | 2598 | CG | ARG | A 343 | 33.686 | 62.471 | 20.031 | 1.00 | 0.00 | C |
| ATOM | 2599 | CD | ARG | A 343 | 33.263 | 63.432 | 21.143 | 1.00 | 0.00 | C |
| ATOM | 2600 | NE | ARG | A 343 | 31.836 | 63.740 | 21.103 | 1.00 | 0.00 | N |
| ATOM | 2601 | CZ | ARG | A 343 | 30.892 | 63.005 | 21.682 | 1.00 | 0.00 | C |
| ATOM | 2602 | NH1 | ARG | A 343 | 31.213 | 61.907 | 22.356 | 1.00 | 0.00 | N |
| ATOM | 2603 | NH2 | ARG | A 343 | 29.621 | 63.374 | 21.591 | 1.00 | 0.00 | N |
| ATOM | 2604 | N | PHE | A 344 | 37.256 | 62.451 | 22.285 | 1.00 | 0.00 | N |
| ATOM | 2605 | CA | PHE | A 344 | 38.351 | 61.999 | 23.136 | 1.00 | 0.00 | C |
| ATOM | 2606 | C | PHE | A 344 | 39.486 | 63.003 | 23.223 | 1.00 | 0.00 | C |
| ATOM | 2607 | O | PHE | A 344 | 40.661 | 62.653 | 23.089 | 1.00 | 0.00 | O |
| ATOM | 2608 | CB | PHE | A 344 | 38.864 | 60.640 | 22.660 | 1.00 | 0.00 | C |
| ATOM | 2609 | CG | PHE | A 344 | 37.858 | 59.544 | 22.816 | 1.00 | 0.00 | C |
| ATOM | 2610 | CD1 | PHE | A 344 | 37.086 | 59.128 | 21.737 | 1.00 | 0.00 | C |
| ATOM | 2611 | CD2 | PHE | A 344 | 37.633 | 58.968 | 24.064 | 1.00 | 0.00 | C |
| ATOM | 2612 | CE1 | PHE | A 344 | 36.103 | 58.158 | 21.896 | 1.00 | 0.00 | C |
| ATOM | 2613 | CE2 | PHE | A 344 | 36.650 | 57.996 | 24.234 | 1.00 | 0.00 | C |
| ATOM | 2614 | CZ | PHE | A 344 | 35.883 | 57.591 | 23.147 | 1.00 | 0.00 | C |
| ATOM | 2615 | N | LYS | A 345 | 39.111 | 64.253 | 23.475 | 1.00 | 0.00 | N |
| ATOM | 2616 | CA | LYS | A 345 | 40.055 | 65.352 | 23.591 | 1.00 | 0.00 | C |
| ATOM | 2617 | C | LYS | A 345 | 40.501 | 65.568 | 25.038 | 1.00 | 0.00 | C |
| ATOM | 2618 | O | LYS | A 345 | 41.691 | 65.581 | 25.328 | 1.00 | 0.00 | O |
| ATOM | 2619 | CB | LYS | A 345 | 39.411 | 66.631 | 23.056 | 1.00 | 0.00 | C |
| ATOM | 2620 | CG | LYS | A 345 | 40.278 | 67.865 | 23.145 | 1.00 | 0.00 | C |
| ATOM | 2621 | CD | LYS | A 345 | 39.504 | 69.083 | 22.658 | 1.00 | 0.00 | C |
| ATOM | 2622 | CE | LYS | A 345 | 40.313 | 70.355 | 22.801 | 1.00 | 0.00 | C |
| ATOM | 2623 | NZ | LYS | A 345 | 39.506 | 71.537 | 22.388 | 1.00 | 0.00 | N |
| ATOM | 2624 | N | GLN | A 346 | 39.540 | 65.728 | 25.941 | 1.00 | 0.00 | N |
| ATOM | 2625 | CA | GLN | A 346 | 39.839 | 65.965 | 27.350 | 1.00 | 0.00 | C |
| ATOM | 2626 | C | GLN | A 346 | 40.008 | 64.681 | 28.158 | 1.00 | 0.00 | C |
| ATOM | 2627 | O | GLN | A 346 | 39.352 | 63.676 | 27.889 | 1.00 | 0.00 | O |
| ATOM | 2628 | CB | GLN | A 346 | 38.722 | 66.800 | 27.973 | 1.00 | 0.00 | C |
| ATOM | 2629 | CG | GLN | A 346 | 38.457 | 68.107 | 27.258 | 1.00 | 0.00 | C |
| ATOM | 2630 | CD | GLN | A 346 | 37.101 | 68.683 | 27.604 | 1.00 | 0.00 | C |
| ATOM | 2631 | OE1 | GLN | A 346 | 36.814 | 68.969 | 28.768 | 1.00 | 0.00 | O |
| ATOM | 2632 | NE2 | GLN | A 346 | 36.252 | 68.850 | 26.593 | 1.00 | 0.00 | N |
| ATOM | 2633 | N | ASN | A 347 | 40.887 | 64.724 | 29.156 | 1.00 | 0.00 | N |
| ATOM | 2634 | CA | ASN | A 347 | 41.120 | 63.566 | 30.012 | 1.00 | 0.00 | C |
| ATOM | 2635 | C | ASN | A 347 | 39.804 | 63.156 | 30.655 | 1.00 | 0.00 | C |
| ATOM | 2636 | O | ASN | A 347 | 39.493 | 61.968 | 30.765 | 1.00 | 0.00 | O |
| ATOM | 2637 | CB | ASN | A 347 | 42.127 | 63.900 | 31.117 | 1.00 | 0.00 | C |
| ATOM | 2638 | CG | ASN | A 347 | 43.499 | 64.205 | 30.577 | 1.00 | 0.00 | C |
| ATOM | 2639 | OD1 | ASN | A 347 | 44.098 | 63.384 | 29.882 | 1.00 | 0.00 | O |
| ATOM | 2640 | ND2 | ASN | A 347 | 44.014 | 65.391 | 30.893 | 1.00 | 0.00 | N |
| ATOM | 2641 | N | THR | A 348 | 39.036 | 64.153 | 31.079 | 1.00 | 0.00 | N |
| ATOM | 2642 | CA | THR | A 348 | 37.751 | 63.910 | 31.716 | 1.00 | 0.00 | C |
| ATOM | 2643 | C | THR | A 348 | 36.837 | 63.127 | 30.785 | 1.00 | 0.00 | C |
| ATOM | 2644 | O | THR | A 348 | 36.042 | 62.300 | 31.237 | 1.00 | 0.00 | O |
| ATOM | 2645 | CB | THR | A 348 | 37.070 | 65.234 | 32.110 | 1.00 | 0.00 | C |
| ATOM | 2646 | OG1 | THR | A 348 | 36.994 | 66.095 | 30.965 | 1.00 | 0.00 | O |
| ATOM | 2647 | CG2 | THR | A 348 | 37.860 | 65.925 | 33.215 | 1.00 | 0.00 | C |
| ATOM | 2648 | N | GLU | A 349 | 36.957 | 63.383 | 29.484 | 1.00 | 0.00 | N |
| ATOM | 2649 | CA | GLU | A 349 | 36.141 | 62.675 | 28.504 | 1.00 | 0.00 | C |
| ATOM | 2650 | C | GLU | A 349 | 36.553 | 61.209 | 28.426 | 1.00 | 0.00 | C |
| ATOM | 2651 | O | GLU | A 349 | 35.701 | 60.324 | 28.382 | 1.00 | 0.00 | O |
| ATOM | 2652 | CB | GLU | A 349 | 36.266 | 63.308 | 27.113 | 1.00 | 0.00 | C |
| ATOM | 2653 | CG | GLU | A 349 | 35.466 | 62.548 | 26.054 | 1.00 | 0.00 | C |
| ATOM | 2654 | CD | GLU | A 349 | 35.592 | 63.132 | 24.659 | 1.00 | 0.00 | C |
| ATOM | 2655 | OE1 | GLU | A 349 | 35.039 | 62.523 | 23.718 | 1.00 | 0.00 | O |
| ATOM | 2656 | OE2 | GLU | A 349 | 36.237 | 64.188 | 24.499 | 1.00 | 0.00 | O |
| ATOM | 2657 | N | TRP | A 350 | 37.859 | 60.950 | 28.396 | 1.00 | 0.00 | N |
| ATOM | 2658 | CA | TRP | A 350 | 38.334 | 59.575 | 28.339 | 1.00 | 0.00 | C |
| ATOM | 2659 | C | TRP | A 350 | 37.800 | 58.804 | 29.539 | 1.00 | 0.00 | C |
| ATOM | 2660 | O | TRP | A 350 | 37.307 | 57.683 | 29.405 | 1.00 | 0.00 | O |
| ATOM | 2661 | CB | TRP | A 350 | 39.865 | 59.515 | 28.342 | 1.00 | 0.00 | C |
| ATOM | 2662 | CG | TRP | A 350 | 40.489 | 59.775 | 27.004 | 1.00 | 0.00 | C |
| ATOM | 2663 | CD1 | TRP | A 350 | 40.855 | 60.987 | 26.485 | 1.00 | 0.00 | C |
| ATOM | 2664 | CD2 | TRP | A 350 | 40.786 | 58.802 | 25.998 | 1.00 | 0.00 | C |
| ATOM | 2665 | NE1 | TRP | A 350 | 41.361 | 60.825 | 25.217 | 1.00 | 0.00 | N |
| ATOM | 2666 | CE2 | TRP | A 350 | 41.329 | 59.495 | 24.892 | 1.00 | 0.00 | C |
| ATOM | 2667 | CE3 | TRP | A 350 | 40.643 | 57.409 | 25.920 | 1.00 | 0.00 | C |
| ATOM | 2668 | CZ2 | TRP | A 350 | 41.729 | 58.842 | 23.722 | 1.00 | 0.00 | C |
| ATOM | 2669 | CZ3 | TRP | A 350 | 41.042 | 56.759 | 24.754 | 1.00 | 0.00 | C |
| ATOM | 2670 | CH2 | TRP | A 350 | 41.578 | 57.477 | 23.672 | 1.00 | 0.00 | C |
| ATOM | 2671 | N | ASP | A 351 | 37.897 | 59.416 | 30.714 | 1.00 | 0.00 | N |
| ATOM | 2672 | CA | ASP | A 351 | 37.427 | 58.778 | 31.937 | 1.00 | 0.00 | C |
| ATOM | 2673 | C | ASP | A 351 | 35.936 | 58.483 | 31.935 | 1.00 | 0.00 | C |
| ATOM | 2674 | O | ASP | A 351 | 35.519 | 57.362 | 32.245 | 1.00 | 0.00 | O |
| ATOM | 2675 | CB | ASP | A 351 | 37.737 | 59.641 | 33.163 | 1.00 | 0.00 | C |
| ATOM | 2676 | CG | ASP | A 351 | 39.211 | 59.698 | 33.484 | 1.00 | 0.00 | C |
| ATOM | 2677 | OD1 | ASP | A 351 | 39.917 | 58.694 | 33.255 | 1.00 | 0.00 | O |
| ATOM | 2678 | OD2 | ASP | A 351 | 39.658 | 60.746 | 33.987 | 1.00 | 0.00 | O |
| ATOM | 2679 | N | VAL | A 352 | 35.131 | 59.484 | 31.593 | 1.00 | 0.00 | N |
| ATOM | 2680 | CA | VAL | A 352 | 33.688 | 59.306 | 31.613 | 1.00 | 0.00 | C |
| ATOM | 2681 | C | VAL | A 352 | 33.197 | 58.234 | 30.637 | 1.00 | 0.00 | C |
| ATOM | 2682 | O | VAL | A 352 | 32.238 | 57.520 | 30.930 | 1.00 | 0.00 | O |
| ATOM | 2683 | CB | VAL | A 352 | 32.951 | 60.650 | 31.362 | 1.00 | 0.00 | C |
| ATOM | 2684 | CG1 | VAL | A 352 | 32.874 | 60.957 | 29.875 | 1.00 | 0.00 | C |
| ATOM | 2685 | CG2 | VAL | A 352 | 31.570 | 60.606 | 31.997 | 1.00 | 0.00 | C |
| ATOM | 2686 | N | GLN | A 353 | 33.848 | 58.106 | 29.484 | 1.00 | 0.00 | N |
| ATOM | 2687 | CA | GLN | A 353 | 33.436 | 57.084 | 28.527 | 1.00 | 0.00 | C |
| ATOM | 2688 | C | GLN | A 353 | 33.959 | 55.709 | 28.960 | 1.00 | 0.00 | C |
| ATOM | 2689 | O | GLN | A 353 | 33.206 | 54.735 | 28.981 | 1.00 | 0.00 | O |
| ATOM | 2690 | CB | GLN | A 353 | 33.942 | 57.422 | 27.114 | 1.00 | 0.00 | C |
| ATOM | 2691 | CG | GLN | A 353 | 33.383 | 58.725 | 26.528 | 1.00 | 0.00 | C |
| ATOM | 2692 | CD | GLN | A 353 | 31.939 | 58.612 | 26.048 | 1.00 | 0.00 | C |
| ATOM | 2693 | OE1 | GLN | A 353 | 31.114 | 57.942 | 26.669 | 1.00 | 0.00 | O |
| ATOM | 2694 | NE2 | GLN | A 353 | 31.626 | 59.289 | 24.943 | 1.00 | 0.00 | N |
| ATOM | 2695 | N | ARG | A 354 | 35.239 | 55.631 | 29.320 | 1.00 | 0.00 | N |
| ATOM | 2696 | CA | ARG | A 354 | 35.834 | 54.359 | 29.734 | 1.00 | 0.00 | C |
| ATOM | 2697 | C | ARG | A 354 | 35.243 | 53.759 | 31.008 | 1.00 | 0.00 | C |
| ATOM | 2698 | O | ARG | A 354 | 34.879 | 52.583 | 31.030 | 1.00 | 0.00 | O |
| ATOM | 2699 | CB | ARG | A 354 | 37.351 | 54.501 | 29.922 | 1.00 | 0.00 | C |
| ATOM | 2700 | CG | ARG | A 354 | 38.018 | 53.237 | 30.465 | 1.00 | 0.00 | C |
| ATOM | 2701 | CD | ARG | A 354 | 39.522 | 53.414 | 30.657 | 1.00 | 0.00 | C |
| ATOM | 2702 | NE | ARG | A 354 | 39.847 | 54.424 | 31.664 | 1.00 | 0.00 | N |
| ATOM | 2703 | CZ | ARG | A 354 | 39.646 | 54.280 | 32.972 | 1.00 | 0.00 | C |
| ATOM | 2704 | NH1 | ARG | A 354 | 39.117 | 53.161 | 33.449 | 1.00 | 0.00 | N |
| ATOM | 2705 | NH2 | ARG | A 354 | 39.983 | 55.254 | 33.806 | 1.00 | 0.00 | N |
| ATOM | 2706 | N | VAL | A 355 | 35.155 | 54.563 | 32.065 | 1.00 | 0.00 | N |
| ATOM | 2707 | CA | VAL | A 355 | 34.634 | 54.082 | 33.343 | 1.00 | 0.00 | C |
| ATOM | 2708 | C | VAL | A 355 | 33.207 | 53.555 | 33.256 | 1.00 | 0.00 | C |
| ATOM | 2709 | O | VAL | A 355 | 32.910 | 52.458 | 33.738 | 1.00 | 0.00 | O |
| ATOM | 2710 | CB | VAL | A 355 | 34.696 | 55.187 | 34.423 | 1.00 | 0.00 | C |
| ATOM | 2711 | CG1 | VAL | A 355 | 34.025 | 54.705 | 35.705 | 1.00 | 0.00 | C |
| ATOM | 2712 | CG2 | VAL | A 355 | 36.151 | 55.549 | 34.709 | 1.00 | 0.00 | C |
| ATOM | 2713 | N | ASN | A 356 | 32.321 | 54.331 | 32.647 | 1.00 | 0.00 | N |
| ATOM | 2714 | CA | ASN | A 356 | 30.940 | 53.901 | 32.525 | 1.00 | 0.00 | C |
| ATOM | 2715 | C | ASN | A 356 | 30.803 | 52.631 | 31.696 | 1.00 | 0.00 | C |
| ATOM | 2716 | O | ASN | A 356 | 30.020 | 51.746 | 32.042 | 1.00 | 0.00 | O |
| ATOM | 2717 | CB | ASN | A 356 | 30.091 | 55.033 | 31.953 | 1.00 | 0.00 | C |
| ATOM | 2718 | CG | ASN | A 356 | 29.785 | 56.094 | 32.990 | 1.00 | 0.00 | C |
| ATOM | 2719 | OD1 | ASN | A 356 | 29.072 | 55.834 | 33.962 | 1.00 | 0.00 | O |
| ATOM | 2720 | ND2 | ASN | A 356 | 30.338 | 57.290 | 32.803 | 1.00 | 0.00 | N |
| ATOM | 2721 | N | TYR | A 357 | 31.568 | 52.522 | 30.613 | 1.00 | 0.00 | N |
| ATOM | 2722 | CA | TYR | A 357 | 31.490 | 51.317 | 29.797 | 1.00 | 0.00 | C |
| ATOM | 2723 | C | TYR | A 357 | 32.099 | 50.109 | 30.507 | 1.00 | 0.00 | C |
| ATOM | 2724 | O | TYR | A 357 | 31.646 | 48.980 | 30.318 | 1.00 | 0.00 | O |
| ATOM | 2725 | CB | TYR | A 357 | 32.144 | 51.538 | 28.427 | 1.00 | 0.00 | C |
| ATOM | 2726 | CG | TYR | A 357 | 31.150 | 52.056 | 27.413 | 1.00 | 0.00 | C |
| ATOM | 2727 | CD1 | TYR | A 357 | 30.957 | 53.425 | 27.225 | 1.00 | 0.00 | C |
| ATOM | 2728 | CD2 | TYR | A 357 | 30.339 | 51.172 | 26.701 | 1.00 | 0.00 | C |
| ATOM | 2729 | CE1 | TYR | A 357 | 29.978 | 53.901 | 26.355 | 1.00 | 0.00 | C |
| ATOM | 2730 | CE2 | TYR | A 357 | 29.355 | 51.635 | 25.835 | 1.00 | 0.00 | C |
| ATOM | 2731 | CZ | TYR | A 357 | 29.177 | 52.999 | 25.666 | 1.00 | 0.00 | C |
| ATOM | 2732 | OH | TYR | A 357 | 28.192 | 53.452 | 24.820 | 1.00 | 0.00 | O |
| ATOM | 2733 | N | GLU | A 358 | 33.115 | 50.339 | 31.330 | 1.00 | 0.00 | N |
| ATOM | 2734 | CA | GLU | A 358 | 33.719 | 49.235 | 32.072 | 1.00 | 0.00 | C |
| ATOM | 2735 | C | GLU | A 358 | 32.679 | 48.654 | 33.037 | 1.00 | 0.00 | C |
| ATOM | 2736 | O | GLU | A 358 | 32.625 | 47.443 | 33.252 | 1.00 | 0.00 | O |
| ATOM | 2737 | CB | GLU | A 358 | 34.938 | 49.712 | 32.863 | 1.00 | 0.00 | C |
| ATOM | 2738 | CG | GLU | A 358 | 36.196 | 49.918 | 32.026 | 1.00 | 0.00 | C |
| ATOM | 2739 | CD | GLU | A 358 | 37.382 | 50.357 | 32.867 | 1.00 | 0.00 | C |
| ATOM | 2740 | OE1 | GLU | A 358 | 37.246 | 50.416 | 34.108 | 1.00 | 0.00 | O |
| ATOM | 2741 | OE2 | GLU | A 358 | 38.453 | 50.640 | 32.293 | 1.00 | 0.00 | O |
| ATOM | 2742 | N | ARG | A 359 | 31.853 | 49.521 | 33.617 | 1.00 | 0.00 | N |
| ATOM | 2743 | CA | ARG | A 359 | 30.816 | 49.068 | 34.545 | 1.00 | 0.00 | C |
| ATOM | 2744 | C | ARG | A 359 | 29.770 | 48.240 | 33.816 | 1.00 | 0.00 | C |
| ATOM | 2745 | O | ARG | A 359 | 29.298 | 47.222 | 34.331 | 1.00 | 0.00 | O |
| ATOM | 2746 | CB | ARG | A 359 | 30.145 | 50.263 | 35.222 | 1.00 | 0.00 | C |
| ATOM | 2747 | CG | ARG | A 359 | 31.033 | 50.954 | 36.227 | 1.00 | 0.00 | C |
| ATOM | 2748 | CD | ARG | A 359 | 30.421 | 52.258 | 36.699 | 1.00 | 0.00 | C |
| ATOM | 2749 | NE | ARG | A 359 | 31.337 | 52.979 | 37.576 | 1.00 | 0.00 | N |
| ATOM | 2750 | CZ | ARG | A 359 | 31.406 | 54.303 | 37.650 | 1.00 | 0.00 | C |
| ATOM | 2751 | NH1 | ARG | A 359 | 30.608 | 55.050 | 36.895 | 1.00 | 0.00 | N |
| ATOM | 2752 | NH2 | ARG | A 359 | 32.278 | 54.880 | 38.471 | 1.00 | 0.00 | N |
| ATOM | 2753 | N | LEU | A 360 | 29.412 | 48.679 | 32.613 | 1.00 | 0.00 | N |
| ATOM | 2754 | CA | LEU | A 360 | 28.429 | 47.965 | 31.810 | 1.00 | 0.00 | C |
| ATOM | 2755 | C | LEU | A 360 | 28.953 | 46.595 | 31.389 | 1.00 | 0.00 | C |
| ATOM | 2756 | O | LEU | A 360 | 28.221 | 45.606 | 31.441 | 1.00 | 0.00 | O |
| ATOM | 2757 | CB | LEU | A 360 | 28.058 | 48.789 | 30.576 | 1.00 | 0.00 | C |
| ATOM | 2758 | CG | LEU | A 360 | 27.224 | 50.045 | 30.849 | 1.00 | 0.00 | C |
| ATOM | 2759 | CD1 | LEU | A 360 | 27.168 | 50.914 | 29.600 | 1.00 | 0.00 | C |
| ATOM | 2760 | CD2 | LEU | A 360 | 25.818 | 49.640 | 31.287 | 1.00 | 0.00 | C |
| ATOM | 2761 | N | PHE | A 361 | 30.217 | 46.537 | 30.975 | 1.00 | 0.00 | N |
| ATOM | 2762 | CA | PHE | A 361 | 30.830 | 45.276 | 30.559 | 1.00 | 0.00 | C |
| ATOM | 2763 | C | PHE | A 361 | 30.896 | 44.266 | 31.706 | 1.00 | 0.00 | C |
| ATOM | 2764 | O | PHE | A 361 | 30.557 | 43.094 | 31.530 | 1.00 | 0.00 | O |
| ATOM | 2765 | CB | PHE | A 361 | 32.252 | 45.513 | 30.035 | 1.00 | 0.00 | C |
| ATOM | 2766 | CG | PHE | A 361 | 32.316 | 46.321 | 28.766 | 1.00 | 0.00 | C |
| ATOM | 2767 | CD1 | PHE | A 361 | 33.510 | 46.923 | 28.376 | 1.00 | 0.00 | C |
| ATOM | 2768 | CD2 | PHE | A 361 | 31.191 | 46.482 | 27.963 | 1.00 | 0.00 | C |
| ATOM | 2769 | CE1 | PHE | A 361 | 33.584 | 47.674 | 27.206 | 1.00 | 0.00 | C |
| ATOM | 2770 | CE2 | PHE | A 361 | 31.253 | 47.232 | 26.787 | 1.00 | 0.00 | C |
| ATOM | 2771 | CZ | PHE | A 361 | 32.451 | 47.828 | 26.410 | 1.00 | 0.00 | C |
| ATOM | 2772 | N | GLU | A 362 | 31.346 | 44.712 | 32.876 | 1.00 | 0.00 | N |
| ATOM | 2773 | CA | GLU | A 362 | 31.456 | 43.803 | 34.013 | 1.00 | 0.00 | C |
| ATOM | 2774 | C | GLU | A 362 | 30.106 | 43.174 | 34.341 | 1.00 | 0.00 | C |
| ATOM | 2775 | O | GLU | A 362 | 30.012 | 41.966 | 34.554 | 1.00 | 0.00 | O |
| ATOM | 2776 | CB | GLU | A 362 | 31.991 | 44.526 | 35.252 | 1.00 | 0.00 | C |
| ATOM | 2777 | CG | GLU | A 362 | 32.334 | 43.567 | 36.393 | 1.00 | 0.00 | C |
| ATOM | 2778 | CD | GLU | A 362 | 32.739 | 44.280 | 37.669 | 1.00 | 0.00 | C |
| ATOM | 2779 | OE1 | GLU | A 362 | 33.486 | 45.274 | 37.581 | 1.00 | 0.00 | O |
| ATOM | 2780 | OE2 | GLU | A 362 | 32.321 | 43.836 | 38.761 | 1.00 | 0.00 | O |
| ATOM | 2781 | N | HIS | A 363 | 29.062 | 43.994 | 34.375 | 1.00 | 0.00 | N |
| ATOM | 2782 | CA | HIS | A 363 | 27.731 | 43.489 | 34.679 | 1.00 | 0.00 | C |
| ATOM | 2783 | C | HIS | A 363 | 27.232 | 42.536 | 33.598 | 1.00 | 0.00 | C |
| ATOM | 2784 | O | HIS | A 363 | 26.919 | 41.376 | 33.869 | 1.00 | 0.00 | O |
| ATOM | 2785 | CB | HIS | A 363 | 26.735 | 44.641 | 34.817 | 1.00 | 0.00 | C |
| ATOM | 2786 | CG | HIS | A 363 | 25.354 | 44.197 | 35.187 | 1.00 | 0.00 | C |
| ATOM | 2787 | ND1 | HIS | A 363 | 25.047 | 43.680 | 36.428 | 1.00 | 0.00 | N |
| ATOM | 2788 | CD2 | HIS | A 363 | 24.205 | 44.159 | 34.469 | 1.00 | 0.00 | C |
| ATOM | 2789 | CE1 | HIS | A 363 | 23.769 | 43.343 | 36.458 | 1.00 | 0.00 | C |
| ATOM | 2790 | NE2 | HIS | A 363 | 23.236 | 43.624 | 35.283 | 1.00 | 0.00 | N |
| ATOM | 2791 | N | ILE | A 364 | 27.159 | 43.037 | 32.371 | 1.00 | 0.00 | N |
| ATOM | 2792 | CA | ILE | A 364 | 26.677 | 42.245 | 31.250 | 1.00 | 0.00 | C |
| ATOM | 2793 | C | ILE | A 364 | 27.396 | 40.910 | 31.084 | 1.00 | 0.00 | C |
| ATOM | 2794 | O | ILE | A 364 | 26.751 | 39.871 | 30.935 | 1.00 | 0.00 | O |
| ATOM | 2795 | CB | ILE | A 364 | 26.782 | 43.039 | 29.931 | 1.00 | 0.00 | C |
| ATOM | 2796 | CG1 | ILE | A 364 | 25.893 | 44.286 | 30.006 | 1.00 | 0.00 | C |
| ATOM | 2797 | CG2 | ILE | A 364 | 26.360 | 42.157 | 28.753 | 1.00 | 0.00 | C |
| ATOM | 2798 | CD1 | ILE | A 364 | 26.022 | 45.206 | 28.813 | 1.00 | 0.00 | C |
| ATOM | 2799 | N | ASN | A 365 | 28.724 | 40.929 | 31.119 | 1.00 | 0.00 | N |
| ATOM | 2800 | CA | ASN | A 365 | 29.489 | 39.699 | 30.943 | 1.00 | 0.00 | C |
| ATOM | 2801 | C | ASN | A 365 | 29.351 | 38.702 | 32.094 | 1.00 | 0.00 | C |
| ATOM | 2802 | O | ASN | A 365 | 29.660 | 37.523 | 31.929 | 1.00 | 0.00 | O |
| ATOM | 2803 | CB | ASN | A 365 | 30.968 | 40.020 | 30.714 | 1.00 | 0.00 | C |
| ATOM | 2804 | CG | ASN | A 365 | 31.189 | 40.887 | 29.488 | 1.00 | 0.00 | C |
| ATOM | 2805 | OD1 | ASN | A 365 | 30.295 | 41.042 | 28.658 | 1.00 | 0.00 | O |
| ATOM | 2806 | ND2 | ASN | A 365 | 32.385 | 41.452 | 29.366 | 1.00 | 0.00 | N |
| ATOM | 2807 | N | SER | A 366 | 28.877 | 39.167 | 33.247 | 1.00 | 0.00 | N |
| ATOM | 2808 | CA | SER | A 366 | 28.710 | 38.292 | 34.407 | 1.00 | 0.00 | C |
| ATOM | 2809 | C | SER | A 366 | 27.261 | 37.837 | 34.581 | 1.00 | 0.00 | C |
| ATOM | 2810 | O | SER | A 366 | 26.957 | 37.032 | 35.458 | 1.00 | 0.00 | O |
| ATOM | 2811 | CB | SER | A 366 | 29.187 | 39.001 | 35.682 | 1.00 | 0.00 | C |
| ATOM | 2812 | OG | SER | A 366 | 28.375 | 40.123 | 35.976 | 1.00 | 0.00 | O |
| ATOM | 2813 | N | GLN | A 367 | 26.370 | 38.361 | 33.745 | 1.00 | 0.00 | N |
| ATOM | 2814 | CA | GLN | A 367 | 24.956 | 37.997 | 33.786 | 1.00 | 0.00 | C |
| ATOM | 2815 | C | GLN | A 367 | 24.661 | 37.028 | 32.642 | 1.00 | 0.00 | C |
| ATOM | 2816 | O | GLN | A 367 | 24.324 | 37.443 | 31.532 | 1.00 | 0.00 | O |
| ATOM | 2817 | CB | GLN | A 367 | 24.077 | 39.244 | 33.646 | 1.00 | 0.00 | C |
| ATOM | 2818 | CG | GLN | A 367 | 24.108 | 40.172 | 34.852 | 1.00 | 0.00 | C |
| ATOM | 2819 | CD | GLN | A 367 | 23.519 | 39.525 | 36.092 | 1.00 | 0.00 | C |
| ATOM | 2820 | OE1 | GLN | A 367 | 22.338 | 39.171 | 36.121 | 1.00 | 0.00 | O |
| ATOM | 2821 | NE2 | GLN | A 367 | 24.342 | 39.362 | 37.122 | 1.00 | 0.00 | N |
| ATOM | 2822 | N | ALA | A 368 | 24.783 | 35.735 | 32.928 | 1.00 | 0.00 | N |
| ATOM | 2823 | CA | ALA | A 368 | 24.557 | 34.686 | 31.937 | 1.00 | 0.00 | C |
| ATOM | 2824 | C | ALA | A 368 | 23.297 | 34.849 | 31.087 | 1.00 | 0.00 | C |
| ATOM | 2825 | O | ALA | A 368 | 23.317 | 34.571 | 29.888 | 1.00 | 0.00 | O |
| ATOM | 2826 | CB | ALA | A 368 | 24.538 | 33.323 | 32.629 | 1.00 | 0.00 | C |
| ATOM | 2827 | N | HIS | A 369 | 22.204 | 35.296 | 31.699 | 1.00 | 0.00 | N |
| ATOM | 2828 | CA | HIS | A 369 | 20.946 | 35.458 | 30.973 | 1.00 | 0.00 | C |
| ATOM | 2829 | C | HIS | A 369 | 21.064 | 36.370 | 29.748 | 1.00 | 0.00 | C |
| ATOM | 2830 | O | HIS | A 369 | 20.220 | 36.328 | 28.852 | 1.00 | 0.00 | O |
| ATOM | 2831 | CB | HIS | A 369 | 19.856 | 35.973 | 31.920 | 1.00 | 0.00 | C |
| ATOM | 2832 | CG | HIS | A 369 | 20.040 | 37.396 | 32.344 | 1.00 | 0.00 | C |
| ATOM | 2833 | ND1 | HIS | A 369 | 19.595 | 38.460 | 31.589 | 1.00 | 0.00 | N |
| ATOM | 2834 | CD2 | HIS | A 369 | 20.636 | 37.932 | 33.435 | 1.00 | 0.00 | C |
| ATOM | 2835 | CE1 | HIS | A 369 | 19.909 | 39.590 | 32.198 | 1.00 | 0.00 | C |
| ATOM | 2836 | NE2 | HIS | A 369 | 20.542 | 39.298 | 33.319 | 1.00 | 0.00 | N |
| ATOM | 2837 | N | PHE | A 370 | 22.107 | 37.193 | 29.707 | 1.00 | 0.00 | N |
| ATOM | 2838 | CA | PHE | A 370 | 22.316 | 38.085 | 28.569 | 1.00 | 0.00 | C |
| ATOM | 2839 | C | PHE | A 370 | 22.956 | 37.314 | 27.422 | 1.00 | 0.00 | C |
| ATOM | 2840 | O | PHE | A 370 | 22.688 | 37.593 | 26.254 | 1.00 | 0.00 | O |
| ATOM | 2841 | CB | PHE | A 370 | 23.233 | 39.252 | 28.952 | 1.00 | 0.00 | C |
| ATOM | 2842 | CG | PHE | A 370 | 22.535 | 40.372 | 29.675 | 1.00 | 0.00 | C |
| ATOM | 2843 | CD1 | PHE | A 370 | 23.086 | 40.916 | 30.829 | 1.00 | 0.00 | C |
| ATOM | 2844 | CD2 | PHE | A 370 | 21.345 | 40.903 | 29.188 | 1.00 | 0.00 | C |
| ATOM | 2845 | CE1 | PHE | A 370 | 22.463 | 41.975 | 31.490 | 1.00 | 0.00 | C |
| ATOM | 2846 | CE2 | PHE | A 370 | 20.713 | 41.964 | 29.842 | 1.00 | 0.00 | C |
| ATOM | 2847 | CZ | PHE | A 370 | 21.277 | 42.499 | 30.995 | 1.00 | 0.00 | C |
| ATOM | 2848 | N | ASN | A 371 | 23.795 | 36.342 | 27.773 | 1.00 | 0.00 | N |
| ATOM | 2849 | CA | ASN | A 371 | 24.506 | 35.530 | 26.791 | 1.00 | 0.00 | C |
| ATOM | 2850 | C | ASN | A 371 | 25.303 | 36.431 | 25.854 | 1.00 | 0.00 | C |
| ATOM | 2851 | O | ASN | A 371 | 25.325 | 36.231 | 24.637 | 1.00 | 0.00 | O |
| ATOM | 2852 | CB | ASN | A 371 | 23.517 | 34.669 | 26.007 | 1.00 | 0.00 | C |
| ATOM | 2853 | CG | ASN | A 371 | 22.867 | 33.607 | 26.875 | 1.00 | 0.00 | C |
| ATOM | 2854 | OD1 | ASN | A 371 | 23.544 | 32.725 | 27.405 | 1.00 | 0.00 | O |
| ATOM | 2855 | ND2 | ASN | A 371 | 21.552 | 33.692 | 27.031 | 1.00 | 0.00 | N |
| ATOM | 2856 | N | VAL | A 372 | 25.961 | 37.421 | 26.451 | 1.00 | 0.00 | N |
| ATOM | 2857 | CA | VAL | A 372 | 26.773 | 38.390 | 25.725 | 1.00 | 0.00 | C |
| ATOM | 2858 | C | VAL | A 372 | 28.142 | 38.559 | 26.374 | 1.00 | 0.00 | C |
| ATOM | 2859 | O | VAL | A 372 | 28.272 | 38.515 | 27.597 | 1.00 | 0.00 | O |
| ATOM | 2860 | CB | VAL | A 372 | 26.092 | 39.785 | 25.702 | 1.00 | 0.00 | C |
| ATOM | 2861 | CG1 | VAL | A 372 | 27.064 | 40.845 | 25.161 | 1.00 | 0.00 | C |
| ATOM | 2862 | CG2 | VAL | A 372 | 24.830 | 39.736 | 24.858 | 1.00 | 0.00 | C |
| ATOM | 2863 | N | GLN | A 373 | 29.157 | 38.748 | 25.539 | 1.00 | 0.00 | N |
| ATOM | 2864 | CA | GLN | A 373 | 30.523 | 38.980 | 25.997 | 1.00 | 0.00 | C |
| ATOM | 2865 | C | GLN | A 373 | 30.933 | 40.267 | 25.284 | 1.00 | 0.00 | C |
| ATOM | 2866 | O | GLN | A 373 | 31.176 | 40.262 | 24.081 | 1.00 | 0.00 | O |
| ATOM | 2867 | CB | GLN | A 373 | 31.438 | 37.822 | 25.583 | 1.00 | 0.00 | C |
| ATOM | 2868 | CG | GLN | A 373 | 32.919 | 38.014 | 25.925 | 1.00 | 0.00 | C |
| ATOM | 2869 | CD | GLN | A 373 | 33.155 | 38.334 | 27.398 | 1.00 | 0.00 | C |
| ATOM | 2870 | OE1 | GLN | A 373 | 32.519 | 37.757 | 28.281 | 1.00 | 0.00 | O |
| ATOM | 2871 | NE2 | GLN | A 373 | 34.082 | 39.248 | 27.665 | 1.00 | 0.00 | N |
| ATOM | 2872 | N | ALA | A 374 | 30.985 | 41.369 | 26.025 | 1.00 | 0.00 | N |
| ATOM | 2873 | CA | ALA | A 374 | 31.329 | 42.660 | 25.444 | 1.00 | 0.00 | C |
| ATOM | 2874 | C | ALA | A 374 | 32.665 | 43.201 | 25.939 | 1.00 | 0.00 | C |
| ATOM | 2875 | O | ALA | A 374 | 33.048 | 42.987 | 27.087 | 1.00 | 0.00 | O |
| ATOM | 2876 | CB | ALA | A 374 | 30.221 | 43.668 | 25.747 | 1.00 | 0.00 | C |
| ATOM | 2877 | N | GLN | A 375 | 33.369 | 43.917 | 25.068 | 1.00 | 0.00 | N |
| ATOM | 2878 | CA | GLN | A 375 | 34.653 | 44.500 | 25.437 | 1.00 | 0.00 | C |
| ATOM | 2879 | C | GLN | A 375 | 35.084 | 45.552 | 24.429 | 1.00 | 0.00 | C |
| ATOM | 2880 | O | GLN | A 375 | 34.526 | 45.641 | 23.336 | 1.00 | 0.00 | O |
| ATOM | 2881 | CB | GLN | A 375 | 35.739 | 43.421 | 25.506 | 1.00 | 0.00 | C |
| ATOM | 2882 | CG | GLN | A 375 | 35.930 | 42.638 | 24.209 | 1.00 | 0.00 | C |
| ATOM | 2883 | CD | GLN | A 375 | 35.048 | 41.409 | 24.145 | 1.00 | 0.00 | C |
| ATOM | 2884 | OE1 | GLN | A 375 | 35.186 | 40.494 | 24.962 | 1.00 | 0.00 | O |
| ATOM | 2885 | NE2 | GLN | A 375 | 34.131 | 41.379 | 23.180 | 1.00 | 0.00 | N |
| ATOM | 2886 | N | PHE | A 376 | 36.070 | 46.359 | 24.810 | 1.00 | 0.00 | N |
| ATOM | 2887 | CA | PHE | A 376 | 36.600 | 47.362 | 23.900 | 1.00 | 0.00 | C |
| ATOM | 2888 | C | PHE | A 376 | 37.395 | 46.571 | 22.874 | 1.00 | 0.00 | C |
| ATOM | 2889 | O | PHE | A 376 | 38.023 | 45.567 | 23.212 | 1.00 | 0.00 | O |
| ATOM | 2890 | CB | PHE | A 376 | 37.540 | 48.327 | 24.630 | 1.00 | 0.00 | C |
| ATOM | 2891 | CG | PHE | A 376 | 36.846 | 49.231 | 25.606 | 1.00 | 0.00 | C |
| ATOM | 2892 | CD1 | PHE | A 376 | 37.245 | 49.273 | 26.936 | 1.00 | 0.00 | C |
| ATOM | 2893 | CD2 | PHE | A 376 | 35.797 | 50.049 | 25.194 | 1.00 | 0.00 | C |
| ATOM | 2894 | CE1 | PHE | A 376 | 36.611 | 50.114 | 27.846 | 1.00 | 0.00 | C |
| ATOM | 2895 | CE2 | PHE | A 376 | 35.156 | 50.896 | 26.100 | 1.00 | 0.00 | C |
| ATOM | 2896 | CZ | PHE | A 376 | 35.567 | 50.926 | 27.429 | 1.00 | 0.00 | C |
| ATOM | 2897 | N | GLY | A 377 | 37.366 | 47.011 | 21.622 | 1.00 | 0.00 | N |
| ATOM | 2898 | CA | GLY | A 377 | 38.115 | 46.309 | 20.598 | 1.00 | 0.00 | C |
| ATOM | 2899 | C | GLY | A 377 | 38.540 | 47.242 | 19.486 | 1.00 | 0.00 | C |
| ATOM | 2900 | O | GLY | A 377 | 38.222 | 48.431 | 19.510 | 1.00 | 0.00 | O |
| ATOM | 2901 | N | THR | A 378 | 39.277 | 46.706 | 18.521 | 1.00 | 0.00 | N |
| ATOM | 2902 | CA | THR | A 378 | 39.716 | 47.493 | 17.380 | 1.00 | 0.00 | C |
| ATOM | 2903 | C | THR | A 378 | 38.965 | 46.972 | 16.164 | 1.00 | 0.00 | C |
| ATOM | 2904 | O | THR | A 378 | 38.287 | 45.940 | 16.235 | 1.00 | 0.00 | O |
| ATOM | 2905 | CB | THR | A 378 | 41.229 | 47.347 | 17.123 | 1.00 | 0.00 | C |
| ATOM | 2906 | OG1 | THR | A 378 | 41.521 | 46.002 | 16.731 | 1.00 | 0.00 | O |
| ATOM | 2907 | CG2 | THR | A 378 | 42.017 | 47.693 | 18.379 | 1.00 | 0.00 | C |
| ATOM | 2908 | N | LEU | A 379 | 39.082 | 47.684 | 15.052 | 1.00 | 0.00 | N |
| ATOM | 2909 | CA | LEU | A 379 | 38.408 | 47.287 | 13.825 | 1.00 | 0.00 | C |
| ATOM | 2910 | C | LEU | A 379 | 38.898 | 45.923 | 13.337 | 1.00 | 0.00 | C |
| ATOM | 2911 | O | LEU | A 379 | 38.102 | 45.072 | 12.931 | 1.00 | 0.00 | O |
| ATOM | 2912 | CB | LEU | A 379 | 38.645 | 48.338 | 12.739 | 1.00 | 0.00 | C |
| ATOM | 2913 | CG | LEU | A 379 | 37.862 | 48.125 | 11.447 | 1.00 | 0.00 | C |
| ATOM | 2914 | CD1 | LEU | A 379 | 36.364 | 48.180 | 11.746 | 1.00 | 0.00 | C |
| ATOM | 2915 | CD2 | LEU | A 379 | 38.261 | 49.198 | 10.432 | 1.00 | 0.00 | C |
| ATOM | 2916 | N | GLN | A 380 | 40.211 | 45.714 | 13.383 | 1.00 | 0.00 | N |
| ATOM | 2917 | CA | GLN | A 380 | 40.783 | 44.449 | 12.936 | 1.00 | 0.00 | C |
| ATOM | 2918 | C | GLN | A 380 | 40.287 | 43.287 | 13.794 | 1.00 | 0.00 | C |
| ATOM | 2919 | O | GLN | A 380 | 40.051 | 42.189 | 13.287 | 1.00 | 0.00 | O |
| ATOM | 2920 | CB | GLN | A 380 | 42.311 | 44.512 | 12.982 | 1.00 | 0.00 | C |
| ATOM | 2921 | CG | GLN | A 380 | 42.989 | 43.294 | 12.377 | 1.00 | 0.00 | C |
| ATOM | 2922 | CD | GLN | A 380 | 42.587 | 43.071 | 10.931 | 1.00 | 0.00 | C |
| ATOM | 2923 | OE1 | GLN | A 380 | 42.667 | 43.983 | 10.104 | 1.00 | 0.00 | O |
| ATOM | 2924 | NE2 | GLN | A 380 | 42.156 | 41.851 | 10.616 | 1.00 | 0.00 | N |
| ATOM | 2925 | N | GLU | A 381 | 40.133 | 43.527 | 15.094 | 1.00 | 0.00 | N |
| ATOM | 2926 | CA | GLU | A 381 | 39.659 | 42.481 | 15.990 | 1.00 | 0.00 | C |
| ATOM | 2927 | C | GLU | A 381 | 38.258 | 42.049 | 15.576 | 1.00 | 0.00 | C |
| ATOM | 2928 | O | GLU | A 381 | 37.941 | 40.862 | 15.576 | 1.00 | 0.00 | O |
| ATOM | 2929 | CB | GLU | A 381 | 39.653 | 42.969 | 17.444 | 1.00 | 0.00 | C |
| ATOM | 2930 | CG | GLU | A 381 | 41.045 | 43.317 | 17.967 | 1.00 | 0.00 | C |
| ATOM | 2931 | CD | GLU | A 381 | 41.068 | 43.599 | 19.460 | 1.00 | 0.00 | C |
| ATOM | 2932 | OE1 | GLU | A 381 | 40.251 | 44.411 | 19.931 | 1.00 | 0.00 | O |
| ATOM | 2933 | OE2 | GLU | A 381 | 41.919 | 43.012 | 20.161 | 1.00 | 0.00 | O |
| ATOM | 2934 | N | TYR | A 382 | 37.420 | 43.019 | 15.225 | 1.00 | 0.00 | N |
| ATOM | 2935 | CA | TYR | A 382 | 36.065 | 42.716 | 14.794 | 1.00 | 0.00 | C |
| ATOM | 2936 | C | TYR | A 382 | 36.081 | 41.821 | 13.554 | 1.00 | 0.00 | C |
| ATOM | 2937 | O | TYR | A 382 | 35.454 | 40.762 | 13.530 | 1.00 | 0.00 | O |
| ATOM | 2938 | CB | TYR | A 382 | 35.300 | 43.999 | 14.464 | 1.00 | 0.00 | C |
| ATOM | 2939 | CG | TYR | A 382 | 33.991 | 43.723 | 13.760 | 1.00 | 0.00 | C |
| ATOM | 2940 | CD1 | TYR | A 382 | 32.937 | 43.095 | 14.429 | 1.00 | 0.00 | C |
| ATOM | 2941 | CD2 | TYR | A 382 | 33.830 | 44.020 | 12.405 | 1.00 | 0.00 | C |
| ATOM | 2942 | CE1 | TYR | A 382 | 31.754 | 42.762 | 13.764 | 1.00 | 0.00 | C |
| ATOM | 2943 | CE2 | TYR | A 382 | 32.654 | 43.691 | 11.731 | 1.00 | 0.00 | C |
| ATOM | 2944 | CZ | TYR | A 382 | 31.622 | 43.058 | 12.418 | 1.00 | 0.00 | C |
| ATOM | 2945 | OH | TYR | A 382 | 30.469 | 42.701 | 11.750 | 1.00 | 0.00 | O |
| ATOM | 2946 | N | PHE | A 383 | 36.791 | 42.257 | 12.519 | 1.00 | 0.00 | N |
| ATOM | 2947 | CA | PHE | A 383 | 36.859 | 41.488 | 11.282 | 1.00 | 0.00 | C |
| ATOM | 2948 | C | PHE | A 383 | 37.454 | 40.088 | 11.470 | 1.00 | 0.00 | C |
| ATOM | 2949 | O | PHE | A 383 | 36.976 | 39.122 | 10.870 | 1.00 | 0.00 | O |
| ATOM | 2950 | CB | PHE | A 383 | 37.648 | 42.269 | 10.220 | 1.00 | 0.00 | C |
| ATOM | 2951 | CG | PHE | A 383 | 36.872 | 43.407 | 9.599 | 1.00 | 0.00 | C |
| ATOM | 2952 | CD1 | PHE | A 383 | 37.365 | 44.705 | 9.641 | 1.00 | 0.00 | C |
| ATOM | 2953 | CD2 | PHE | A 383 | 35.655 | 43.173 | 8.962 | 1.00 | 0.00 | C |
| ATOM | 2954 | CE1 | PHE | A 383 | 36.662 | 45.762 | 9.057 | 1.00 | 0.00 | C |
| ATOM | 2955 | CE2 | PHE | A 383 | 34.938 | 44.218 | 8.372 | 1.00 | 0.00 | C |
| ATOM | 2956 | CZ | PHE | A 383 | 35.444 | 45.518 | 8.419 | 1.00 | 0.00 | C |
| ATOM | 2957 | N | ASP | A 384 | 38.490 | 39.968 | 12.297 | 1.00 | 0.00 | N |
| ATOM | 2958 | CA | ASP | A 384 | 39.094 | 38.655 | 12.535 | 1.00 | 0.00 | C |
| ATOM | 2959 | C | ASP | A 384 | 38.050 | 37.702 | 13.109 | 1.00 | 0.00 | C |
| ATOM | 2960 | O | ASP | A 384 | 37.958 | 36.545 | 12.699 | 1.00 | 0.00 | O |
| ATOM | 2961 | CB | ASP | A 384 | 40.266 | 38.753 | 13.514 | 1.00 | 0.00 | C |
| ATOM | 2962 | CG | ASP | A 384 | 41.499 | 39.382 | 12.897 | 1.00 | 0.00 | C |
| ATOM | 2963 | OD1 | ASP | A 384 | 41.577 | 39.469 | 11.652 | 1.00 | 0.00 | O |
| ATOM | 2964 | OD2 | ASP | A 384 | 42.400 | 39.775 | 13.667 | 1.00 | 0.00 | O |
| ATOM | 2965 | N | ALA | A 385 | 37.261 | 38.198 | 14.057 | 1.00 | 0.00 | N |
| ATOM | 2966 | CA | ALA | A 385 | 36.223 | 37.391 | 14.691 | 1.00 | 0.00 | C |
| ATOM | 2967 | C | ALA | A 385 | 35.143 | 37.007 | 13.684 | 1.00 | 0.00 | C |
| ATOM | 2968 | O | ALA | A 385 | 34.686 | 35.863 | 13.652 | 1.00 | 0.00 | O |
| ATOM | 2969 | CB | ALA | A 385 | 35.605 | 38.155 | 15.862 | 1.00 | 0.00 | C |
| ATOM | 2970 | N | VAL | A 386 | 34.731 | 37.965 | 12.862 | 1.00 | 0.00 | N |
| ATOM | 2971 | CA | VAL | A 386 | 33.715 | 37.695 | 11.851 | 1.00 | 0.00 | C |
| ATOM | 2972 | C | VAL | A 386 | 34.149 | 36.546 | 10.939 | 1.00 | 0.00 | C |
| ATOM | 2973 | O | VAL | A 386 | 33.385 | 35.608 | 10.689 | 1.00 | 0.00 | O |
| ATOM | 2974 | CB | VAL | A 386 | 33.447 | 38.938 | 10.982 | 1.00 | 0.00 | C |
| ATOM | 2975 | CG1 | VAL | A 386 | 32.555 | 38.564 | 9.794 | 1.00 | 0.00 | C |
| ATOM | 2976 | CG2 | VAL | A 386 | 32.782 | 40.018 | 11.823 | 1.00 | 0.00 | C |
| ATOM | 2977 | N | HIS | A 387 | 35.379 | 36.612 | 10.447 | 1.00 | 0.00 | N |
| ATOM | 2978 | CA | HIS | A 387 | 35.867 | 35.570 | 9.562 | 1.00 | 0.00 | C |
| ATOM | 2979 | C | HIS | A 387 | 36.092 | 34.244 | 10.280 | 1.00 | 0.00 | C |
| ATOM | 2980 | O | HIS | A 387 | 36.062 | 33.181 | 9.657 | 1.00 | 0.00 | O |
| ATOM | 2981 | CB | HIS | A 387 | 37.125 | 36.055 | 8.843 | 1.00 | 0.00 | C |
| ATOM | 2982 | CG | HIS | A 387 | 36.852 | 37.170 | 7.882 | 1.00 | 0.00 | C |
| ATOM | 2983 | ND1 | HIS | A 387 | 36.009 | 37.024 | 6.800 | 1.00 | 0.00 | N |
| ATOM | 2984 | CD2 | HIS | A 387 | 37.263 | 38.461 | 7.867 | 1.00 | 0.00 | C |
| ATOM | 2985 | CE1 | HIS | A 387 | 35.912 | 38.177 | 6.162 | 1.00 | 0.00 | C |
| ATOM | 2986 | NE2 | HIS | A 387 | 36.663 | 39.065 | 6.790 | 1.00 | 0.00 | N |
| ATOM | 2987 | N | GLN | A 388 | 36.304 | 34.303 | 11.590 | 1.00 | 0.00 | N |
| ATOM | 2988 | CA | GLN | A 388 | 36.474 | 33.083 | 12.367 | 1.00 | 0.00 | C |
| ATOM | 2989 | C | GLN | A 388 | 35.105 | 32.397 | 12.401 | 1.00 | 0.00 | C |
| ATOM | 2990 | O | GLN | A 388 | 35.007 | 31.173 | 12.317 | 1.00 | 0.00 | O |
| ATOM | 2991 | CB | GLN | A 388 | 36.943 | 33.411 | 13.789 | 1.00 | 0.00 | C |
| ATOM | 2992 | CG | GLN | A 388 | 38.442 | 33.684 | 13.910 | 1.00 | 0.00 | C |
| ATOM | 2993 | CD | GLN | A 388 | 38.816 | 34.342 | 15.235 | 1.00 | 0.00 | C |
| ATOM | 2994 | OE1 | GLN | A 388 | 38.248 | 34.024 | 16.281 | 1.00 | 0.00 | O |
| ATOM | 2995 | NE2 | GLN | A 388 | 39.783 | 35.256 | 15.194 | 1.00 | 0.00 | N |
| ATOM | 2996 | N | ALA | A 389 | 34.049 | 33.199 | 12.508 | 1.00 | 0.00 | N |
| ATOM | 2997 | CA | ALA | A 389 | 32.686 | 32.674 | 12.536 | 1.00 | 0.00 | C |
| ATOM | 2998 | C | ALA | A 389 | 32.342 | 32.106 | 11.163 | 1.00 | 0.00 | C |
| ATOM | 2999 | O | ALA | A 389 | 31.688 | 31.064 | 11.050 | 1.00 | 0.00 | O |
| ATOM | 3000 | CB | ALA | A 389 | 31.705 | 33.780 | 12.911 | 1.00 | 0.00 | C |
| ATOM | 3001 | N | GLU | A 390 | 32.792 | 32.803 | 10.125 | 1.00 | 0.00 | N |
| ATOM | 3002 | CA | GLU | A 390 | 32.565 | 32.398 | 8.743 | 1.00 | 0.00 | C |
| ATOM | 3003 | C | GLU | A 390 | 33.197 | 31.033 | 8.487 | 1.00 | 0.00 | C |
| ATOM | 3004 | O | GLU | A 390 | 32.569 | 30.144 | 7.910 | 1.00 | 0.00 | O |
| ATOM | 3005 | CB | GLU | A 390 | 33.168 | 33.445 | 7.798 | 1.00 | 0.00 | C |
| ATOM | 3006 | CG | GLU | A 390 | 33.255 | 33.036 | 6.331 | 1.00 | 0.00 | C |
| ATOM | 3007 | CD | GLU | A 390 | 33.859 | 34.133 | 5.464 | 1.00 | 0.00 | C |
| ATOM | 3008 | OE1 | GLU | A 390 | 34.757 | 34.847 | 5.956 | 1.00 | 0.00 | O |
| ATOM | 3009 | OE2 | GLU | A 390 | 33.447 | 34.275 | 4.292 | 1.00 | 0.00 | O |
| ATOM | 3010 | N | ARG | A 391 | 34.443 | 30.876 | 8.921 | 1.00 | 0.00 | N |
| ATOM | 3011 | CA | ARG | A 391 | 35.163 | 29.622 | 8.746 | 1.00 | 0.00 | C |
| ATOM | 3012 | C | ARG | A 391 | 34.519 | 28.504 | 9.560 | 1.00 | 0.00 | C |
| ATOM | 3013 | O | ARG | A 391 | 34.611 | 27.330 | 9.200 | 1.00 | 0.00 | O |
| ATOM | 3014 | CB | ARG | A 391 | 36.628 | 29.790 | 9.160 | 1.00 | 0.00 | C |
| ATOM | 3015 | CG | ARG | A 391 | 37.475 | 30.565 | 8.159 | 1.00 | 0.00 | C |
| ATOM | 3016 | CD | ARG | A 391 | 38.908 | 30.719 | 8.652 | 1.00 | 0.00 | C |
| ATOM | 3017 | NE | ARG | A 391 | 38.993 | 31.636 | 9.785 | 1.00 | 0.00 | N |
| ATOM | 3018 | CZ | ARG | A 391 | 40.086 | 31.827 | 10.515 | 1.00 | 0.00 | C |
| ATOM | 3019 | NH1 | ARG | A 391 | 41.201 | 31.162 | 10.238 | 1.00 | 0.00 | N |
| ATOM | 3020 | NH2 | ARG | A 391 | 40.066 | 32.688 | 11.524 | 1.00 | 0.00 | N |
| ATOM | 3021 | N | ALA | A 392 | 33.870 | 28.871 | 10.660 | 1.00 | 0.00 | N |
| ATOM | 3022 | CA | ALA | A 392 | 33.204 | 27.891 | 11.510 | 1.00 | 0.00 | C |
| ATOM | 3023 | C | ALA | A 392 | 31.932 | 27.419 | 10.814 | 1.00 | 0.00 | C |
| ATOM | 3024 | O | ALA | A 392 | 31.255 | 26.500 | 11.281 | 1.00 | 0.00 | O |
| ATOM | 3025 | CB | ALA | A 392 | 32.870 | 28.508 | 12.863 | 1.00 | 0.00 | C |
| ATOM | 3026 | N | GLY | A 393 | 31.612 | 28.064 | 9.695 | 1.00 | 0.00 | N |
| ATOM | 3027 | CA | GLY | A 393 | 30.432 | 27.701 | 8.935 | 1.00 | 0.00 | C |
| ATOM | 3028 | C | GLY | A 393 | 29.122 | 28.243 | 9.473 | 1.00 | 0.00 | C |
| ATOM | 3029 | O | GLY | A 393 | 28.063 | 27.676 | 9.206 | 1.00 | 0.00 | O |
| ATOM | 3030 | N | GLN | A 394 | 29.170 | 29.338 | 10.225 | 1.00 | 0.00 | N |
| ATOM | 3031 | CA | GLN | A 394 | 27.937 | 29.897 | 10.760 | 1.00 | 0.00 | C |
| ATOM | 3032 | C | GLN | A 394 | 27.377 | 31.012 | 9.883 | 1.00 | 0.00 | C |
| ATOM | 3033 | O | GLN | A 394 | 26.321 | 31.566 | 10.180 | 1.00 | 0.00 | O |
| ATOM | 3034 | CB | GLN | A 394 | 28.140 | 30.410 | 12.191 | 1.00 | 0.00 | C |
| ATOM | 3035 | CG | GLN | A 394 | 28.877 | 31.728 | 12.312 | 1.00 | 0.00 | C |
| ATOM | 3036 | CD | GLN | A 394 | 28.757 | 32.324 | 13.707 | 1.00 | 0.00 | C |
| ATOM | 3037 | OE1 | GLN | A 394 | 29.229 | 31.746 | 14.689 | 1.00 | 0.00 | O |
| ATOM | 3038 | NE2 | GLN | A 394 | 28.114 | 33.483 | 13.799 | 1.00 | 0.00 | N |
| ATOM | 3039 | N | ALA | A 395 | 28.072 | 31.333 | 8.795 | 1.00 | 0.00 | N |
| ATOM | 3040 | CA | ALA | A 395 | 27.606 | 32.383 | 7.898 | 1.00 | 0.00 | C |
| ATOM | 3041 | C | ALA | A 395 | 28.251 | 32.344 | 6.517 | 1.00 | 0.00 | C |
| ATOM | 3042 | O | ALA | A 395 | 29.421 | 31.995 | 6.369 | 1.00 | 0.00 | O |
| ATOM | 3043 | CB | ALA | A 395 | 27.840 | 33.750 | 8.538 | 1.00 | 0.00 | C |
| ATOM | 3044 | N | GLU | A 396 | 27.463 | 32.700 | 5.509 | 1.00 | 0.00 | N |
| ATOM | 3045 | CA | GLU | A 396 | 27.921 | 32.752 | 4.126 | 1.00 | 0.00 | C |
| ATOM | 3046 | C | GLU | A 396 | 27.614 | 34.180 | 3.696 | 1.00 | 0.00 | C |
| ATOM | 3047 | O | GLU | A 396 | 26.528 | 34.688 | 3.980 | 1.00 | 0.00 | O |
| ATOM | 3048 | CB | GLU | A 396 | 27.135 | 31.766 | 3.256 | 1.00 | 0.00 | C |
| ATOM | 3049 | CG | GLU | A 396 | 26.450 | 30.641 | 4.026 | 1.00 | 0.00 | C |
| ATOM | 3050 | CD | GLU | A 396 | 25.095 | 31.053 | 4.591 | 1.00 | 0.00 | C |
| ATOM | 3051 | OE1 | GLU | A 396 | 25.037 | 32.008 | 5.400 | 1.00 | 0.00 | O |
| ATOM | 3052 | OE2 | GLU | A 396 | 24.083 | 30.417 | 4.221 | 1.00 | 0.00 | O |
| ATOM | 3053 | N | PHE | A 397 | 28.553 | 34.835 | 3.025 | 1.00 | 0.00 | N |
| ATOM | 3054 | CA | PHE | A 397 | 28.321 | 36.215 | 2.620 | 1.00 | 0.00 | C |
| ATOM | 3055 | C | PHE | A 397 | 27.997 | 36.401 | 1.147 | 1.00 | 0.00 | C |
| ATOM | 3056 | O | PHE | A 397 | 28.569 | 35.744 | 0.281 | 1.00 | 0.00 | O |
| ATOM | 3057 | CB | PHE | A 397 | 29.520 | 37.087 | 2.999 | 1.00 | 0.00 | C |
| ATOM | 3058 | CG | PHE | A 397 | 29.773 | 37.145 | 4.477 | 1.00 | 0.00 | C |
| ATOM | 3059 | CD1 | PHE | A 397 | 30.811 | 36.417 | 5.047 | 1.00 | 0.00 | C |
| ATOM | 3060 | CD2 | PHE | A 397 | 28.952 | 37.905 | 5.303 | 1.00 | 0.00 | C |
| ATOM | 3061 | CE1 | PHE | A 397 | 31.030 | 36.444 | 6.429 | 1.00 | 0.00 | C |
| ATOM | 3062 | CE2 | PHE | A 397 | 29.160 | 37.940 | 6.682 | 1.00 | 0.00 | C |
| ATOM | 3063 | CZ | PHE | A 397 | 30.201 | 37.206 | 7.245 | 1.00 | 0.00 | C |
| ATOM | 3064 | N | PRO | A 398 | 27.070 | 37.322 | 0.849 | 1.00 | 0.00 | N |
| ATOM | 3065 | CA | PRO | A 398 | 26.654 | 37.611 | −0.522 | 1.00 | 0.00 | C |
| ATOM | 3066 | C | PRO | A 398 | 27.725 | 38.385 | −1.291 | 1.00 | 0.00 | C |
| ATOM | 3067 | O | PRO | A 398 | 28.587 | 39.037 | −0.692 | 1.00 | 0.00 | O |
| ATOM | 3068 | CB | PRO | A 398 | 25.386 | 38.428 | −0.318 | 1.00 | 0.00 | C |
| ATOM | 3069 | CG | PRO | A 398 | 25.728 | 39.233 | 0.898 | 1.00 | 0.00 | C |
| ATOM | 3070 | CD | PRO | A 398 | 26.358 | 38.190 | 1.806 | 1.00 | 0.00 | C |
| ATOM | 3071 | N | THR | A 399 | 27.666 | 38.294 | −2.616 | 1.00 | 0.00 | N |
| ATOM | 3072 | CA | THR | A 399 | 28.598 | 38.994 | −3.490 | 1.00 | 0.00 | C |
| ATOM | 3073 | C | THR | A 399 | 27.876 | 40.240 | −3.990 | 1.00 | 0.00 | C |
| ATOM | 3074 | O | THR | A 399 | 26.647 | 40.260 | −4.077 | 1.00 | 0.00 | O |
| ATOM | 3075 | CB | THR | A 399 | 29.000 | 38.127 | −4.694 | 1.00 | 0.00 | C |
| ATOM | 3076 | OG1 | THR | A 399 | 27.821 | 37.692 | −5.387 | 1.00 | 0.00 | O |
| ATOM | 3077 | CG2 | THR | A 399 | 29.798 | 36.923 | −4.231 | 1.00 | 0.00 | C |
| ATOM | 3078 | N | LEU | A 400 | 28.632 | 41.280 | −4.318 | 1.00 | 0.00 | N |
| ATOM | 3079 | CA | LEU | A 400 | 28.017 | 42.519 | −4.779 | 1.00 | 0.00 | C |
| ATOM | 3080 | C | LEU | A 400 | 28.941 | 43.319 | −5.680 | 1.00 | 0.00 | C |
| ATOM | 3081 | O | LEU | A 400 | 30.160 | 43.292 | −5.511 | 1.00 | 0.00 | O |
| ATOM | 3082 | CB | LEU | A 400 | 27.615 | 43.364 | −3.560 | 1.00 | 0.00 | C |
| ATOM | 3083 | CG | LEU | A 400 | 26.959 | 44.742 | −3.738 | 1.00 | 0.00 | C |
| ATOM | 3084 | CD1 | LEU | A 400 | 26.173 | 45.078 | −2.487 | 1.00 | 0.00 | C |
| ATOM | 3085 | CD2 | LEU | A 400 | 28.008 | 45.816 | −4.013 | 1.00 | 0.00 | C |
| ATOM | 3086 | N | SER | A 401 | 28.354 | 44.003 | −6.659 | 1.00 | 0.00 | N |
| ATOM | 3087 | CA | SER | A 401 | 29.115 | 44.862 | −7.562 | 1.00 | 0.00 | C |
| ATOM | 3088 | C | SER | A 401 | 28.278 | 46.126 | −7.742 | 1.00 | 0.00 | C |
| ATOM | 3089 | O | SER | A 401 | 27.057 | 46.092 | −7.574 | 1.00 | 0.00 | O |
| ATOM | 3090 | CB | SER | A 401 | 29.352 | 44.191 | −8.922 | 1.00 | 0.00 | C |
| ATOM | 3091 | OG | SER | A 401 | 28.184 | 44.208 | −9.721 | 1.00 | 0.00 | O |
| ATOM | 3092 | N | GLY | A 402 | 28.937 | 47.234 | −8.073 | 1.00 | 0.00 | N |
| ATOM | 3093 | CA | GLY | A 402 | 28.238 | 48.495 | −8.259 | 1.00 | 0.00 | C |
| ATOM | 3094 | C | GLY | A 402 | 28.819 | 49.577 | −7.364 | 1.00 | 0.00 | C |
| ATOM | 3095 | O | GLY | A 402 | 29.842 | 49.360 | −6.715 | 1.00 | 0.00 | O |
| ATOM | 3096 | N | ASP | A 403 | 28.178 | 50.741 | −7.325 | 1.00 | 0.00 | N |
| ATOM | 3097 | CA | ASP | A 403 | 28.661 | 51.833 | −6.489 | 1.00 | 0.00 | C |
| ATOM | 3098 | C | ASP | A 403 | 27.554 | 52.332 | −5.568 | 1.00 | 0.00 | C |
| ATOM | 3099 | O | ASP | A 403 | 26.442 | 51.795 | −5.567 | 1.00 | 0.00 | O |
| ATOM | 3100 | CB | ASP | A 403 | 29.179 | 52.983 | −7.364 | 1.00 | 0.00 | C |
| ATOM | 3101 | CG | ASP | A 403 | 28.065 | 53.735 | −8.070 | 1.00 | 0.00 | C |
| ATOM | 3102 | OD1 | ASP | A 403 | 26.966 | 53.170 | −8.232 | 1.00 | 0.00 | O |
| ATOM | 3103 | OD2 | ASP | A 403 | 28.294 | 54.893 | −8.475 | 1.00 | 0.00 | O |
| ATOM | 3104 | N | PHE | A 404 | 27.862 | 53.355 | −4.780 | 1.00 | 0.00 | N |
| ATOM | 3105 | CA | PHE | A 404 | 26.886 | 53.913 | −3.863 | 1.00 | 0.00 | C |
| ATOM | 3106 | C | PHE | A 404 | 26.735 | 55.422 | −4.002 | 1.00 | 0.00 | C |
| ATOM | 3107 | O | PHE | A 404 | 26.906 | 56.178 | −3.046 | 1.00 | 0.00 | O |
| ATOM | 3108 | CB | PHE | A 404 | 27.234 | 53.526 | −2.421 | 1.00 | 0.00 | C |
| ATOM | 3109 | CG | PHE | A 404 | 27.207 | 52.036 | −2.182 | 1.00 | 0.00 | C |
| ATOM | 3110 | CD1 | PHE | A 404 | 28.380 | 51.288 | −2.212 | 1.00 | 0.00 | C |
| ATOM | 3111 | CD2 | PHE | A 404 | 25.997 | 51.378 | −1.976 | 1.00 | 0.00 | C |
| ATOM | 3112 | CE1 | PHE | A 404 | 28.350 | 49.899 | −2.041 | 1.00 | 0.00 | C |
| ATOM | 3113 | CE2 | PHE | A 404 | 25.953 | 49.990 | −1.804 | 1.00 | 0.00 | C |
| ATOM | 3114 | CZ | PHE | A 404 | 27.130 | 49.249 | −1.836 | 1.00 | 0.00 | C |
| ATOM | 3115 | N | PHE | A 405 | 26.421 | 55.834 | −5.228 | 1.00 | 0.00 | N |
| ATOM | 3116 | CA | PHE | A 405 | 26.172 | 57.229 | −5.575 | 1.00 | 0.00 | C |
| ATOM | 3117 | C | PHE | A 405 | 24.818 | 57.168 | −6.290 | 1.00 | 0.00 | C |
| ATOM | 3118 | O | PHE | A 405 | 24.541 | 56.185 | −6.975 | 1.00 | 0.00 | O |
| ATOM | 3119 | CB | PHE | A 405 | 27.236 | 57.749 | −6.549 | 1.00 | 0.00 | C |
| ATOM | 3120 | CG | PHE | A 405 | 28.613 | 57.857 | −5.955 | 1.00 | 0.00 | C |
| ATOM | 3121 | CD1 | PHE | A 405 | 29.700 | 57.254 | −6.584 | 1.00 | 0.00 | C |
| ATOM | 3122 | CD2 | PHE | A 405 | 28.834 | 58.583 | −4.786 | 1.00 | 0.00 | C |
| ATOM | 3123 | CE1 | PHE | A 405 | 30.988 | 57.373 | −6.055 | 1.00 | 0.00 | C |
| ATOM | 3124 | CE2 | PHE | A 405 | 30.121 | 58.707 | −4.251 | 1.00 | 0.00 | C |
| ATOM | 3125 | CZ | PHE | A 405 | 31.196 | 58.101 | −4.888 | 1.00 | 0.00 | C |
| ATOM | 3126 | N | THR | A 406 | 23.973 | 58.189 | −6.153 | 1.00 | 0.00 | N |
| ATOM | 3127 | CA | THR | A 406 | 24.252 | 59.380 | −5.363 | 1.00 | 0.00 | C |
| ATOM | 3128 | C | THR | A 406 | 23.610 | 59.282 | −3.982 | 1.00 | 0.00 | C |
| ATOM | 3129 | O | THR | A 406 | 22.440 | 58.917 | −3.834 | 1.00 | 0.00 | O |
| ATOM | 3130 | CB | THR | A 406 | 23.746 | 60.643 | −6.110 | 1.00 | 0.00 | C |
| ATOM | 3131 | OG1 | THR | A 406 | 24.634 | 60.928 | −7.200 | 1.00 | 0.00 | O |
| ATOM | 3132 | CG2 | THR | A 406 | 23.673 | 61.847 | −5.180 | 1.00 | 0.00 | C |
| ATOM | 3133 | N | TYR | A 407 | 24.405 | 59.608 | −2.971 | 1.00 | 0.00 | N |
| ATOM | 3134 | CA | TYR | A 407 | 23.987 | 59.564 | −1.577 | 1.00 | 0.00 | C |
| ATOM | 3135 | C | TYR | A 407 | 22.894 | 60.561 | −1.209 | 1.00 | 0.00 | C |
| ATOM | 3136 | O | TYR | A 407 | 22.834 | 61.669 | −1.742 | 1.00 | 0.00 | O |
| ATOM | 3137 | CB | TYR | A 407 | 25.209 | 59.811 | −0.696 | 1.00 | 0.00 | C |
| ATOM | 3138 | CG | TYR | A 407 | 24.941 | 59.975 | 0.786 | 1.00 | 0.00 | C |
| ATOM | 3139 | CD1 | TYR | A 407 | 24.453 | 58.918 | 1.554 | 1.00 | 0.00 | C |
| ATOM | 3140 | CD2 | TYR | A 407 | 25.277 | 61.163 | 1.437 | 1.00 | 0.00 | C |
| ATOM | 3141 | CE1 | TYR | A 407 | 24.321 | 59.037 | 2.941 | 1.00 | 0.00 | C |
| ATOM | 3142 | CE2 | TYR | A 407 | 25.151 | 61.293 | 2.813 | 1.00 | 0.00 | C |
| ATOM | 3143 | CZ | TYR | A 407 | 24.681 | 60.229 | 3.562 | 1.00 | 0.00 | C |
| ATOM | 3144 | OH | TYR | A 407 | 24.630 | 60.348 | 4.932 | 1.00 | 0.00 | O |
| ATOM | 3145 | N | ALA | A 408 | 22.032 | 60.137 | −0.291 | 1.00 | 0.00 | N |
| ATOM | 3146 | CA | ALA | A 408 | 20.950 | 60.958 | 0.239 | 1.00 | 0.00 | C |
| ATOM | 3147 | C | ALA | A 408 | 20.844 | 60.512 | 1.689 | 1.00 | 0.00 | C |
| ATOM | 3148 | O | ALA | A 408 | 20.689 | 59.319 | 1.956 | 1.00 | 0.00 | O |
| ATOM | 3149 | CB | ALA | A 408 | 19.640 | 60.684 | −0.502 | 1.00 | 0.00 | C |
| ATOM | 3150 | N | ASP | A 409 | 20.959 | 61.449 | 2.626 | 1.00 | 0.00 | N |
| ATOM | 3151 | CA | ASP | A 409 | 20.881 | 61.086 | 4.035 | 1.00 | 0.00 | C |
| ATOM | 3152 | C | ASP | A 409 | 19.456 | 61.137 | 4.575 | 1.00 | 0.00 | C |
| ATOM | 3153 | O | ASP | A 409 | 19.160 | 60.548 | 5.613 | 1.00 | 0.00 | O |
| ATOM | 3154 | CB | ASP | A 409 | 21.834 | 61.959 | 4.877 | 1.00 | 0.00 | C |
| ATOM | 3155 | CG | ASP | A 409 | 21.513 | 63.444 | 4.808 | 1.00 | 0.00 | C |
| ATOM | 3156 | OD1 | ASP | A 409 | 20.928 | 63.893 | 3.802 | 1.00 | 0.00 | O |
| ATOM | 3157 | OD2 | ASP | A 409 | 21.875 | 64.169 | 5.765 | 1.00 | 0.00 | O |
| ATOM | 3158 | N | ARG | A 410 | 18.574 | 61.824 | 3.853 | 1.00 | 0.00 | N |
| ATOM | 3159 | CA | ARG | A 410 | 17.167 | 61.928 | 4.238 | 1.00 | 0.00 | C |
| ATOM | 3160 | C | ARG | A 410 | 16.320 | 62.561 | 3.137 | 1.00 | 0.00 | C |
| ATOM | 3161 | O | ARG | A 410 | 16.824 | 63.319 | 2.306 | 1.00 | 0.00 | O |
| ATOM | 3162 | CB | ARG | A 410 | 17.008 | 62.732 | 5.533 | 1.00 | 0.00 | C |
| ATOM | 3163 | CG | ARG | A 410 | 17.450 | 64.189 | 5.473 | 1.00 | 0.00 | C |
| ATOM | 3164 | CD | ARG | A 410 | 17.319 | 64.801 | 6.863 | 1.00 | 0.00 | C |
| ATOM | 3165 | NE | ARG | A 410 | 17.959 | 66.107 | 7.001 | 1.00 | 0.00 | N |
| ATOM | 3166 | CZ | ARG | A 410 | 17.440 | 67.255 | 6.578 | 1.00 | 0.00 | C |
| ATOM | 3167 | NH1 | ARG | A 410 | 16.255 | 67.277 | 5.977 | 1.00 | 0.00 | N |
| ATOM | 3168 | NH2 | ARG | A 410 | 18.108 | 68.386 | 6.768 | 1.00 | 0.00 | N |
| ATOM | 3169 | N | SER | A 411 | 15.031 | 62.230 | 3.146 | 1.00 | 0.00 | N |
| ATOM | 3170 | CA | SER | A 411 | 14.062 | 62.737 | 2.179 | 1.00 | 0.00 | C |
| ATOM | 3171 | C | SER | A 411 | 14.587 | 62.849 | 0.751 | 1.00 | 0.00 | C |
| ATOM | 3172 | O | SER | A 411 | 15.017 | 61.855 | 0.160 | 1.00 | 0.00 | O |
| ATOM | 3173 | CB | SER | A 411 | 13.512 | 64.094 | 2.645 | 1.00 | 0.00 | C |
| ATOM | 3174 | OG | SER | A 411 | 14.555 | 65.012 | 2.916 | 1.00 | 0.00 | O |
| ATOM | 3175 | N | ASP | A 412 | 14.534 | 64.055 | 0.195 | 1.00 | 0.00 | N |
| ATOM | 3176 | CA | ASP | A 412 | 15.000 | 64.292 | −1.166 | 1.00 | 0.00 | C |
| ATOM | 3177 | C | ASP | A 412 | 16.345 | 65.016 | −1.175 | 1.00 | 0.00 | C |
| ATOM | 3178 | O | ASP | A 412 | 16.756 | 65.551 | −2.200 | 1.00 | 0.00 | O |
| ATOM | 3179 | CB | ASP | A 412 | 13.966 | 65.122 | −1.941 | 1.00 | 0.00 | C |
| ATOM | 3180 | CG | ASP | A 412 | 13.832 | 66.548 | −1.410 | 1.00 | 0.00 | C |
| ATOM | 3181 | OD1 | ASP | A 412 | 14.316 | 66.826 | −0.291 | 1.00 | 0.00 | O |
| ATOM | 3182 | OD2 | ASP | A 412 | 13.228 | 67.389 | −2.114 | 1.00 | 0.00 | O |
| ATOM | 3183 | N | ASN | A 413 | 17.022 | 65.027 | −0.030 | 1.00 | 0.00 | N |
| ATOM | 3184 | CA | ASN | A 413 | 18.316 | 65.699 | 0.088 | 1.00 | 0.00 | C |
| ATOM | 3185 | C | ASN | A 413 | 19.452 | 64.858 | −0.492 | 1.00 | 0.00 | C |
| ATOM | 3186 | O | ASN | A 413 | 20.149 | 64.160 | 0.248 | 1.00 | 0.00 | O |
| ATOM | 3187 | CB | ASN | A 413 | 18.637 | 66.012 | 1.553 | 1.00 | 0.00 | C |
| ATOM | 3188 | CG | ASN | A 413 | 17.727 | 67.076 | 2.156 | 1.00 | 0.00 | C |
| ATOM | 3189 | OD1 | ASN | A 413 | 17.963 | 67.536 | 3.272 | 1.00 | 0.00 | O |
| ATOM | 3190 | ND2 | ASN | A 413 | 16.687 | 67.464 | 1.429 | 1.00 | 0.00 | N |
| ATOM | 3191 | N | TYR | A 414 | 19.631 | 64.928 | −1.809 | 1.00 | 0.00 | N |
| ATOM | 3192 | CA | TYR | A 414 | 20.696 | 64.187 | −2.483 | 1.00 | 0.00 | C |
| ATOM | 3193 | C | TYR | A 414 | 21.940 | 65.067 | −2.556 | 1.00 | 0.00 | C |
| ATOM | 3194 | O | TYR | A 414 | 21.869 | 66.235 | −2.942 | 1.00 | 0.00 | O |
| ATOM | 3195 | CB | TYR | A 414 | 20.264 | 63.763 | −3.891 | 1.00 | 0.00 | C |
| ATOM | 3196 | CG | TYR | A 414 | 19.261 | 62.630 | −3.900 | 1.00 | 0.00 | C |
| ATOM | 3197 | CD1 | TYR | A 414 | 17.909 | 62.859 | −3.628 | 1.00 | 0.00 | C |
| ATOM | 3198 | CD2 | TYR | A 414 | 19.668 | 61.319 | −4.152 | 1.00 | 0.00 | C |
| ATOM | 3199 | CE1 | TYR | A 414 | 16.989 | 61.806 | −3.609 | 1.00 | 0.00 | C |
| ATOM | 3200 | CE2 | TYR | A 414 | 18.760 | 60.262 | −4.132 | 1.00 | 0.00 | C |
| ATOM | 3201 | CZ | TYR | A 414 | 17.423 | 60.511 | −3.861 | 1.00 | 0.00 | C |
| ATOM | 3202 | OH | TYR | A 414 | 16.528 | 59.462 | −3.852 | 1.00 | 0.00 | O |
| ATOM | 3203 | N | TRP | A 415 | 23.078 | 64.488 | −2.190 | 1.00 | 0.00 | N |
| ATOM | 3204 | CA | TRP | A 415 | 24.343 | 65.211 | −2.153 | 1.00 | 0.00 | C |
| ATOM | 3205 | C | TRP | A 415 | 25.086 | 65.255 | −3.482 | 1.00 | 0.00 | C |
| ATOM | 3206 | O | TRP | A 415 | 26.225 | 64.807 | −3.568 | 1.00 | 0.00 | O |
| ATOM | 3207 | CB | TRP | A 415 | 25.251 | 64.586 | −1.092 | 1.00 | 0.00 | C |
| ATOM | 3208 | CG | TRP | A 415 | 24.680 | 64.608 | 0.294 | 1.00 | 0.00 | C |
| ATOM | 3209 | CD1 | TRP | A 415 | 23.384 | 64.351 | 0.658 | 1.00 | 0.00 | C |
| ATOM | 3210 | CD2 | TRP | A 415 | 25.394 | 64.860 | 1.507 | 1.00 | 0.00 | C |
| ATOM | 3211 | NE1 | TRP | A 415 | 23.251 | 64.428 | 2.023 | 1.00 | 0.00 | N |
| ATOM | 3212 | CE2 | TRP | A 415 | 24.470 | 64.738 | 2.570 | 1.00 | 0.00 | C |
| ATOM | 3213 | CE3 | TRP | A 415 | 26.729 | 65.175 | 1.802 | 1.00 | 0.00 | C |
| ATOM | 3214 | CZ2 | TRP | A 415 | 24.837 | 64.920 | 3.907 | 1.00 | 0.00 | C |
| ATOM | 3215 | CZ3 | TRP | A 415 | 27.095 | 65.355 | 3.128 | 1.00 | 0.00 | C |
| ATOM | 3216 | CH2 | TRP | A 415 | 26.150 | 65.226 | 4.167 | 1.00 | 0.00 | C |
| ATOM | 3217 | N | SER | A 416 | 24.452 | 65.796 | −4.516 | 1.00 | 0.00 | N |
| ATOM | 3218 | CA | SER | A 416 | 25.100 | 65.881 | −5.814 | 1.00 | 0.00 | C |
| ATOM | 3219 | C | SER | A 416 | 25.676 | 67.274 | −6.048 | 1.00 | 0.00 | C |
| ATOM | 3220 | O | SER | A 416 | 26.310 | 67.528 | −7.065 | 1.00 | 0.00 | O |
| ATOM | 3221 | CB | SER | A 416 | 24.115 | 65.510 | −6.931 | 1.00 | 0.00 | C |
| ATOM | 3222 | OG | SER | A 416 | 22.842 | 66.097 | −6.719 | 1.00 | 0.00 | O |
| ATOM | 3223 | N | GLY | A 417 | 25.465 | 68.171 | −5.089 | 1.00 | 0.00 | N |
| ATOM | 3224 | CA | GLY | A 417 | 25.979 | 69.524 | −5.223 | 1.00 | 0.00 | C |
| ATOM | 3225 | C | GLY | A 417 | 27.495 | 69.591 | −5.153 | 1.00 | 0.00 | C |
| ATOM | 3226 | O | GLY | A 417 | 28.126 | 70.313 | −5.928 | 1.00 | 0.00 | O |
| ATOM | 3227 | N | TYR | A 418 | 28.084 | 68.821 | −4.241 | 1.00 | 0.00 | N |
| ATOM | 3228 | CA | TYR | A 418 | 29.534 | 68.815 | −4.057 | 1.00 | 0.00 | C |
| ATOM | 3229 | C | TYR | A 418 | 30.308 | 68.208 | −5.234 | 1.00 | 0.00 | C |
| ATOM | 3230 | O | TYR | A 418 | 31.539 | 68.209 | −5.250 | 1.00 | 0.00 | O |
| ATOM | 3231 | CB | TYR | A 418 | 29.894 | 68.109 | −2.743 | 1.00 | 0.00 | C |
| ATOM | 3232 | CG | TYR | A 418 | 30.042 | 66.601 | −2.814 | 1.00 | 0.00 | C |
| ATOM | 3233 | CD1 | TYR | A 418 | 31.295 | 66.014 | −2.998 | 1.00 | 0.00 | C |
| ATOM | 3234 | CD2 | TYR | A 418 | 28.941 | 65.760 | −2.636 | 1.00 | 0.00 | C |
| ATOM | 3235 | CE1 | TYR | A 418 | 31.452 | 64.622 | −2.993 | 1.00 | 0.00 | C |
| ATOM | 3236 | CE2 | TYR | A 418 | 29.087 | 64.363 | −2.631 | 1.00 | 0.00 | C |
| ATOM | 3237 | CZ | TYR | A 418 | 30.346 | 63.808 | −2.807 | 1.00 | 0.00 | C |
| ATOM | 3238 | OH | TYR | A 418 | 30.509 | 62.440 | −2.778 | 1.00 | 0.00 | O |
| ATOM | 3239 | N | TYR | A 419 | 29.588 | 67.682 | −6.218 | 1.00 | 0.00 | N |
| ATOM | 3240 | CA | TYR | A 419 | 30.247 | 67.143 | −7.400 | 1.00 | 0.00 | C |
| ATOM | 3241 | C | TYR | A 419 | 30.767 | 68.352 | −8.190 | 1.00 | 0.00 | C |
| ATOM | 3242 | O | TYR | A 419 | 31.607 | 68.207 | −9.082 | 1.00 | 0.00 | O |
| ATOM | 3243 | CB | TYR | A 419 | 29.255 | 66.377 | −8.286 | 1.00 | 0.00 | C |
| ATOM | 3244 | CG | TYR | A 419 | 28.627 | 65.145 | −7.665 | 1.00 | 0.00 | C |
| ATOM | 3245 | CD1 | TYR | A 419 | 27.472 | 64.589 | −8.213 | 1.00 | 0.00 | C |
| ATOM | 3246 | CD2 | TYR | A 419 | 29.197 | 64.518 | −6.557 | 1.00 | 0.00 | C |
| ATOM | 3247 | CE1 | TYR | A 419 | 26.900 | 63.439 | −7.677 | 1.00 | 0.00 | C |
| ATOM | 3248 | CE2 | TYR | A 419 | 28.633 | 63.362 | −6.012 | 1.00 | 0.00 | C |
| ATOM | 3249 | CZ | TYR | A 419 | 27.485 | 62.829 | −6.580 | 1.00 | 0.00 | C |
| ATOM | 3250 | OH | TYR | A 419 | 26.927 | 61.678 | −6.064 | 1.00 | 0.00 | O |
| ATOM | 3251 | N | THR | A 420 | 30.279 | 69.544 | −7.840 | 1.00 | 0.00 | N |
| ATOM | 3252 | CA | THR | A 420 | 30.661 | 70.770 | −8.546 | 1.00 | 0.00 | C |
| ATOM | 3253 | C | THR | A 420 | 31.221 | 71.919 | −7.697 | 1.00 | 0.00 | C |
| ATOM | 3254 | O | THR | A 420 | 32.026 | 72.712 | −8.187 | 1.00 | 0.00 | O |
| ATOM | 3255 | CB | THR | A 420 | 29.455 | 71.310 | −9.354 | 1.00 | 0.00 | C |
| ATOM | 3256 | OG1 | THR | A 420 | 28.971 | 70.281 | −10.227 | 1.00 | 0.00 | O |
| ATOM | 3257 | CG2 | THR | A 420 | 29.854 | 72.529 | −10.190 | 1.00 | 0.00 | C |
| ATOM | 3258 | N | SER | A 421 | 30.800 | 72.008 | −6.436 | 1.00 | 0.00 | N |
| ATOM | 3259 | CA | SER | A 421 | 31.242 | 73.076 | −5.536 | 1.00 | 0.00 | C |
| ATOM | 3260 | C | SER | A 421 | 32.735 | 73.389 | −5.606 | 1.00 | 0.00 | C |
| ATOM | 3261 | O | SER | A 421 | 33.572 | 72.482 | −5.614 | 1.00 | 0.00 | O |
| ATOM | 3262 | CB | SER | A 421 | 30.857 | 72.735 | −4.094 | 1.00 | 0.00 | C |
| ATOM | 3263 | OG | SER | A 421 | 29.453 | 72.577 | −3.981 | 1.00 | 0.00 | O |
| ATOM | 3264 | N | ARG | A 422 | 33.053 | 74.684 | −5.635 | 1.00 | 0.00 | N |
| ATOM | 3265 | CA | ARG | A 422 | 34.435 | 75.157 | −5.717 | 1.00 | 0.00 | C |
| ATOM | 3266 | C | ARG | A 422 | 35.159 | 74.483 | −6.885 | 1.00 | 0.00 | C |
| ATOM | 3267 | O | ARG | A 422 | 36.171 | 73.796 | −6.706 | 1.00 | 0.00 | O |
| ATOM | 3268 | CB | ARG | A 422 | 35.165 | 74.882 | −4.399 | 1.00 | 0.00 | C |
| ATOM | 3269 | CG | ARG | A 422 | 34.962 | 75.955 | −3.314 | 1.00 | 0.00 | C |
| ATOM | 3270 | CD | ARG | A 422 | 33.503 | 76.181 | −2.910 | 1.00 | 0.00 | C |
| ATOM | 3271 | NE | ARG | A 422 | 33.428 | 77.149 | −1.812 | 1.00 | 0.00 | N |
| ATOM | 3272 | CZ | ARG | A 422 | 33.509 | 76.833 | −0.522 | 1.00 | 0.00 | C |
| ATOM | 3273 | NH1 | ARG | A 422 | 33.643 | 75.570 | −0.148 | 1.00 | 0.00 | N |
| ATOM | 3274 | NH2 | ARG | A 422 | 33.518 | 77.790 | 0.400 | 1.00 | 0.00 | N |
| ATOM | 3275 | N | PRO | A 423 | 34.657 | 74.698 | −8.111 | 1.00 | 0.00 | N |
| ATOM | 3276 | CA | PRO | A 423 | 35.246 | 74.107 | −9.316 | 1.00 | 0.00 | C |
| ATOM | 3277 | C | PRO | A 423 | 36.676 | 74.537 | −9.638 | 1.00 | 0.00 | C |
| ATOM | 3278 | O | PRO | A 423 | 37.406 | 73.810 | −10.309 | 1.00 | 0.00 | O |
| ATOM | 3279 | CB | PRO | A 423 | 34.249 | 74.490 | −10.409 | 1.00 | 0.00 | C |
| ATOM | 3280 | CG | PRO | A 423 | 33.751 | 75.827 | −9.945 | 1.00 | 0.00 | C |
| ATOM | 3281 | CD | PRO | A 423 | 33.540 | 75.596 | −8.457 | 1.00 | 0.00 | C |
| ATOM | 3282 | N | TYR | A 424 | 37.077 | 75.717 | −9.175 | 1.00 | 0.00 | N |
| ATOM | 3283 | CA | TYR | A 424 | 38.434 | 76.193 | −9.433 | 1.00 | 0.00 | C |
| ATOM | 3284 | C | TYR | A 424 | 39.447 | 75.199 | −8.863 | 1.00 | 0.00 | C |
| ATOM | 3285 | O | TYR | A 424 | 40.399 | 74.793 | −9.536 | 1.00 | 0.00 | O |
| ATOM | 3286 | CB | TYR | A 424 | 38.654 | 77.556 | −8.770 | 1.00 | 0.00 | C |
| ATOM | 3287 | CG | TYR | A 424 | 40.023 | 78.142 | −9.032 | 1.00 | 0.00 | C |
| ATOM | 3288 | CD1 | TYR | A 424 | 40.264 | 78.904 | −10.171 | 1.00 | 0.00 | C |
| ATOM | 3289 | CD2 | TYR | A 424 | 41.082 | 77.922 | −8.146 | 1.00 | 0.00 | C |
| ATOM | 3290 | CE1 | TYR | A 424 | 41.522 | 79.439 | −10.425 | 1.00 | 0.00 | C |
| ATOM | 3291 | CE2 | TYR | A 424 | 42.351 | 78.452 | −8.393 | 1.00 | 0.00 | C |
| ATOM | 3292 | CZ | TYR | A 424 | 42.561 | 79.212 | −9.536 | 1.00 | 0.00 | C |
| ATOM | 3293 | OH | TYR | A 424 | 43.801 | 79.754 | −9.797 | 1.00 | 0.00 | O |
| ATOM | 3294 | N | HIS | A 425 | 39.223 | 74.798 | −7.617 | 1.00 | 0.00 | N |
| ATOM | 3295 | CA | HIS | A 425 | 40.121 | 73.876 | −6.933 | 1.00 | 0.00 | C |
| ATOM | 3296 | C | HIS | A 425 | 39.983 | 72.441 | −7.433 | 1.00 | 0.00 | C |
| ATOM | 3297 | O | HIS | A 425 | 40.940 | 71.665 | −7.375 | 1.00 | 0.00 | O |
| ATOM | 3298 | CB | HIS | A 425 | 39.872 | 73.988 | −5.432 | 1.00 | 0.00 | C |
| ATOM | 3299 | CG | HIS | A 425 | 39.848 | 75.407 | −4.958 | 1.00 | 0.00 | C |
| ATOM | 3300 | ND1 | HIS | A 425 | 40.997 | 76.127 | −4.708 | 1.00 | 0.00 | N |
| ATOM | 3301 | CD2 | HIS | A 425 | 38.821 | 76.281 | −4.824 | 1.00 | 0.00 | C |
| ATOM | 3302 | CE1 | HIS | A 425 | 40.681 | 77.382 | −4.446 | 1.00 | 0.00 | C |
| ATOM | 3303 | NE2 | HIS | A 425 | 39.367 | 77.503 | −4.511 | 1.00 | 0.00 | N |
| ATOM | 3304 | N | LYS | A 426 | 38.799 | 72.091 | −7.925 | 1.00 | 0.00 | N |
| ATOM | 3305 | CA | LYS | A 426 | 38.581 | 70.755 | −8.480 | 1.00 | 0.00 | C |
| ATOM | 3306 | C | LYS | A 426 | 39.470 | 70.629 | −9.719 | 1.00 | 0.00 | C |
| ATOM | 3307 | O | LYS | A 426 | 40.072 | 69.577 | −9.968 | 1.00 | 0.00 | O |
| ATOM | 3308 | CB | LYS | A 426 | 37.107 | 70.563 | −8.870 | 1.00 | 0.00 | C |
| ATOM | 3309 | CG | LYS | A 426 | 36.195 | 70.152 | −7.716 | 1.00 | 0.00 | C |
| ATOM | 3310 | CD | LYS | A 426 | 34.725 | 70.105 | −8.148 | 1.00 | 0.00 | C |
| ATOM | 3311 | CE | LYS | A 426 | 33.869 | 69.286 | −7.178 | 1.00 | 0.00 | C |
| ATOM | 3312 | NZ | LYS | A 426 | 33.884 | 69.803 | −5.776 | 1.00 | 0.00 | N |
| ATOM | 3313 | N | ARG | A 427 | 39.551 | 71.709 | −10.494 | 1.00 | 0.00 | N |
| ATOM | 3314 | CA | ARG | A 427 | 40.382 | 71.732 | −11.694 | 1.00 | 0.00 | C |
| ATOM | 3315 | C | ARG | A 427 | 41.850 | 71.769 | −11.269 | 1.00 | 0.00 | C |
| ATOM | 3316 | O | ARG | A 427 | 42.695 | 71.093 | −11.852 | 1.00 | 0.00 | O |
| ATOM | 3317 | CB | ARG | A 427 | 40.024 | 72.955 | −12.555 | 1.00 | 0.00 | C |
| ATOM | 3318 | CG | ARG | A 427 | 41.003 | 73.283 | −13.676 | 1.00 | 0.00 | C |
| ATOM | 3319 | CD | ARG | A 427 | 41.277 | 72.113 | −14.624 | 1.00 | 0.00 | C |
| ATOM | 3320 | NE | ARG | A 427 | 42.272 | 72.510 | −15.617 | 1.00 | 0.00 | N |
| ATOM | 3321 | CZ | ARG | A 427 | 43.045 | 71.672 | −16.298 | 1.00 | 0.00 | C |
| ATOM | 3322 | NH1 | ARG | A 427 | 42.951 | 70.361 | −16.110 | 1.00 | 0.00 | N |
| ATOM | 3323 | NH2 | ARG | A 427 | 43.936 | 72.155 | −17.155 | 1.00 | 0.00 | N |
| ATOM | 3324 | N | MET | A 428 | 42.145 | 72.544 | −10.233 | 1.00 | 0.00 | N |
| ATOM | 3325 | CA | MET | A 428 | 43.514 | 72.641 | −9.739 | 1.00 | 0.00 | C |
| ATOM | 3326 | C | MET | A 428 | 44.018 | 71.255 | −9.321 | 1.00 | 0.00 | C |
| ATOM | 3327 | O | MET | A 428 | 45.196 | 70.928 | −9.507 | 1.00 | 0.00 | O |
| ATOM | 3328 | CB | MET | A 428 | 43.570 | 73.610 | −8.555 | 1.00 | 0.00 | C |
| ATOM | 3329 | CG | MET | A 428 | 44.976 | 73.929 | −8.088 | 1.00 | 0.00 | C |
| ATOM | 3330 | SD | MET | A 428 | 45.002 | 75.301 | −6.918 | 1.00 | 0.00 | S |
| ATOM | 3331 | CE | MET | A 428 | 46.758 | 75.634 | −6.852 | 1.00 | 0.00 | C |
| ATOM | 3332 | N | ASP | A 429 | 43.117 | 70.438 | −8.775 | 1.00 | 0.00 | N |
| ATOM | 3333 | CA | ASP | A 429 | 43.473 | 69.085 | −8.348 | 1.00 | 0.00 | C |
| ATOM | 3334 | C | ASP | A 429 | 44.061 | 68.268 | −9.492 | 1.00 | 0.00 | C |
| ATOM | 3335 | O | ASP | A 429 | 45.083 | 67.600 | −9.328 | 1.00 | 0.00 | O |
| ATOM | 3336 | CB | ASP | A 429 | 42.247 | 68.356 | −7.785 | 1.00 | 0.00 | C |
| ATOM | 3337 | CG | ASP | A 429 | 42.531 | 66.893 | −7.463 | 1.00 | 0.00 | C |
| ATOM | 3338 | OD1 | ASP | A 429 | 42.221 | 66.022 | −8.309 | 1.00 | 0.00 | O |
| ATOM | 3339 | OD2 | ASP | A 429 | 43.071 | 66.617 | −6.370 | 1.00 | 0.00 | O |
| ATOM | 3340 | N | ARG | A 430 | 43.415 | 68.328 | −10.653 | 1.00 | 0.00 | N |
| ATOM | 3341 | CA | ARG | A 430 | 43.874 | 67.574 | −11.817 | 1.00 | 0.00 | C |
| ATOM | 3342 | C | ARG | A 430 | 45.199 | 68.087 | −12.368 | 1.00 | 0.00 | C |
| ATOM | 3343 | O | ARG | A 430 | 46.017 | 67.312 | −12.877 | 1.00 | 0.00 | O |
| ATOM | 3344 | CB | ARG | A 430 | 42.803 | 67.606 | −12.907 | 1.00 | 0.00 | C |
| ATOM | 3345 | CG | ARG | A 430 | 41.512 | 66.914 | −12.504 | 1.00 | 0.00 | C |
| ATOM | 3346 | CD | ARG | A 430 | 41.748 | 65.447 | −12.150 | 1.00 | 0.00 | C |
| ATOM | 3347 | NE | ARG | A 430 | 40.503 | 64.684 | −12.126 | 1.00 | 0.00 | N |
| ATOM | 3348 | CZ | ARG | A 430 | 39.819 | 64.378 | −11.027 | 1.00 | 0.00 | C |
| ATOM | 3349 | NH1 | ARG | A 430 | 40.252 | 64.760 | −9.832 | 1.00 | 0.00 | N |
| ATOM | 3350 | NH2 | ARG | A 430 | 38.683 | 63.694 | −11.126 | 1.00 | 0.00 | N |
| ATOM | 3351 | N | VAL | A 431 | 45.407 | 69.395 | −12.278 | 1.00 | 0.00 | N |
| ATOM | 3352 | CA | VAL | A 431 | 46.651 | 69.989 | −12.748 | 1.00 | 0.00 | C |
| ATOM | 3353 | C | VAL | A 431 | 47.789 | 69.514 | −11.840 | 1.00 | 0.00 | C |
| ATOM | 3354 | O | VAL | A 431 | 48.817 | 69.038 | −12.315 | 1.00 | 0.00 | O |
| ATOM | 3355 | CB | VAL | A 431 | 46.579 | 71.536 | −12.721 | 1.00 | 0.00 | C |
| ATOM | 3356 | CG1 | VAL | A 431 | 47.951 | 72.135 | −13.040 | 1.00 | 0.00 | C |
| ATOM | 3357 | CG2 | VAL | A 431 | 45.550 | 72.024 | −13.735 | 1.00 | 0.00 | C |
| ATOM | 3358 | N | LEU | A 432 | 47.599 | 69.630 | −10.530 | 1.00 | 0.00 | N |
| ATOM | 3359 | CA | LEU | A 432 | 48.635 | 69.205 | −9.595 | 1.00 | 0.00 | C |
| ATOM | 3360 | C | LEU | A 432 | 48.864 | 67.703 | −9.699 | 1.00 | 0.00 | C |
| ATOM | 3361 | O | LEU | A 432 | 49.996 | 67.234 | −9.588 | 1.00 | 0.00 | O |
| ATOM | 3362 | CB | LEU | A 432 | 48.258 | 69.586 | −8.160 | 1.00 | 0.00 | C |
| ATOM | 3363 | CG | LEU | A 432 | 49.291 | 69.259 | −7.078 | 1.00 | 0.00 | C |
| ATOM | 3364 | CD1 | LEU | A 432 | 50.658 | 69.838 | −7.461 | 1.00 | 0.00 | C |
| ATOM | 3365 | CD2 | LEU | A 432 | 48.823 | 69.839 | −5.736 | 1.00 | 0.00 | C |
| ATOM | 3366 | N | MET | A 433 | 47.792 | 66.946 | −9.918 | 1.00 | 0.00 | N |
| ATOM | 3367 | CA | MET | A 433 | 47.919 | 65.497 | −10.062 | 1.00 | 0.00 | C |
| ATOM | 3368 | C | MET | A 433 | 48.970 | 65.194 | −11.120 | 1.00 | 0.00 | C |
| ATOM | 3369 | O | MET | A 433 | 49.852 | 64.351 | −10.920 | 1.00 | 0.00 | O |
| ATOM | 3370 | CB | MET | A 433 | 46.594 | 64.873 | −10.502 | 1.00 | 0.00 | C |
| ATOM | 3371 | CG | MET | A 433 | 46.728 | 63.412 | −10.918 | 1.00 | 0.00 | C |
| ATOM | 3372 | SD | MET | A 433 | 45.192 | 62.708 | −11.557 | 1.00 | 0.00 | S |
| ATOM | 3373 | CE | MET | A 433 | 45.225 | 63.322 | −13.248 | 1.00 | 0.00 | C |
| ATOM | 3374 | N | HIS | A 434 | 48.869 | 65.886 | −12.250 | 1.00 | 0.00 | N |
| ATOM | 3375 | CA | HIS | A 434 | 49.806 | 65.680 | −13.346 | 1.00 | 0.00 | C |
| ATOM | 3376 | C | HIS | A 434 | 51.207 | 66.194 | −13.049 | 1.00 | 0.00 | C |
| ATOM | 3377 | O | HIS | A 434 | 52.196 | 65.539 | −13.386 | 1.00 | 0.00 | O |
| ATOM | 3378 | CB | HIS | A 434 | 49.293 | 66.333 | −14.631 | 1.00 | 0.00 | C |
| ATOM | 3379 | CG | HIS | A 434 | 50.274 | 66.259 | −15.755 | 1.00 | 0.00 | C |
| ATOM | 3380 | ND1 | HIS | A 434 | 51.015 | 67.345 | −16.167 | 1.00 | 0.00 | N |
| ATOM | 3381 | CD2 | HIS | A 434 | 50.712 | 65.205 | −16.484 | 1.00 | 0.00 | C |
| ATOM | 3382 | CE1 | HIS | A 434 | 51.869 | 66.961 | −17.099 | 1.00 | 0.00 | C |
| ATOM | 3383 | NE2 | HIS | A 434 | 51.707 | 65.668 | −17.310 | 1.00 | 0.00 | N |
| ATOM | 3384 | N | TYR | A 435 | 51.294 | 67.372 | −12.435 | 1.00 | 0.00 | N |
| ATOM | 3385 | CA | TYR | A 435 | 52.593 | 67.943 | −12.100 | 1.00 | 0.00 | C |
| ATOM | 3386 | C | TYR | A 435 | 53.359 | 67.019 | −11.162 | 1.00 | 0.00 | C |
| ATOM | 3387 | O | TYR | A 435 | 54.573 | 66.871 | −11.292 | 1.00 | 0.00 | O |
| ATOM | 3388 | CB | TYR | A 435 | 52.432 | 69.315 | −11.435 | 1.00 | 0.00 | C |
| ATOM | 3389 | CG | TYR | A 435 | 52.316 | 70.482 | −12.393 | 1.00 | 0.00 | C |
| ATOM | 3390 | CD1 | TYR | A 435 | 51.335 | 70.513 | −13.384 | 1.00 | 0.00 | C |
| ATOM | 3391 | CD2 | TYR | A 435 | 53.160 | 71.587 | −12.267 | 1.00 | 0.00 | C |
| ATOM | 3392 | CE1 | TYR | A 435 | 51.193 | 71.624 | −14.226 | 1.00 | 0.00 | C |
| ATOM | 3393 | CE2 | TYR | A 435 | 53.028 | 72.697 | −13.097 | 1.00 | 0.00 | C |
| ATOM | 3394 | CZ | TYR | A 435 | 52.043 | 72.711 | −14.072 | 1.00 | 0.00 | C |
| ATOM | 3395 | OH | TYR | A 435 | 51.898 | 73.830 | −14.866 | 1.00 | 0.00 | O |
| ATOM | 3396 | N | VAL | A 436 | 52.656 | 66.400 | −10.215 | 1.00 | 0.00 | N |
| ATOM | 3397 | CA | VAL | A 436 | 53.317 | 65.490 | −9.282 | 1.00 | 0.00 | C |
| ATOM | 3398 | C | VAL | A 436 | 53.886 | 64.293 | −10.038 | 1.00 | 0.00 | C |
| ATOM | 3399 | O | VAL | A 436 | 55.042 | 63.912 | −9.841 | 1.00 | 0.00 | O |
| ATOM | 3400 | CB | VAL | A 436 | 52.345 | 65.011 | −8.173 | 1.00 | 0.00 | C |
| ATOM | 3401 | CG1 | VAL | A 436 | 52.927 | 63.816 | −7.437 | 1.00 | 0.00 | C |
| ATOM | 3402 | CG2 | VAL | A 436 | 52.098 | 66.149 | −7.186 | 1.00 | 0.00 | C |
| ATOM | 3403 | N | ARG | A 437 | 53.079 | 63.708 | −10.916 | 1.00 | 0.00 | N |
| ATOM | 3404 | CA | ARG | A 437 | 53.540 | 62.566 | −11.696 | 1.00 | 0.00 | C |
| ATOM | 3405 | C | ARG | A 437 | 54.754 | 62.934 | −12.546 | 1.00 | 0.00 | C |
| ATOM | 3406 | O | ARG | A 437 | 55.739 | 62.194 | −12.594 | 1.00 | 0.00 | O |
| ATOM | 3407 | CB | ARG | A 437 | 52.420 | 62.048 | −12.606 | 1.00 | 0.00 | C |
| ATOM | 3408 | CG | ARG | A 437 | 52.918 | 61.115 | −13.703 | 1.00 | 0.00 | C |
| ATOM | 3409 | CD | ARG | A 437 | 51.782 | 60.539 | −14.543 | 1.00 | 0.00 | C |
| ATOM | 3410 | NE | ARG | A 437 | 52.290 | 59.871 | −15.741 | 1.00 | 0.00 | N |
| ATOM | 3411 | CZ | ARG | A 437 | 51.563 | 59.066 | −16.513 | 1.00 | 0.00 | C |
| ATOM | 3412 | NH1 | ARG | A 437 | 50.293 | 58.819 | −16.213 | 1.00 | 0.00 | N |
| ATOM | 3413 | NH2 | ARG | A 437 | 52.099 | 58.518 | −17.595 | 1.00 | 0.00 | N |
| ATOM | 3414 | N | ALA | A 438 | 54.681 | 64.077 | −13.221 | 1.00 | 0.00 | N |
| ATOM | 3415 | CA | ALA | A 438 | 55.772 | 64.522 | −14.086 | 1.00 | 0.00 | C |
| ATOM | 3416 | C | ALA | A 438 | 57.053 | 64.806 | −13.308 | 1.00 | 0.00 | C |
| ATOM | 3417 | O | ALA | A 438 | 58.149 | 64.467 | −13.760 | 1.00 | 0.00 | O |
| ATOM | 3418 | CB | ALA | A 438 | 55.345 | 65.762 | −14.878 | 1.00 | 0.00 | C |
| ATOM | 3419 | N | ALA | A 439 | 56.914 | 65.418 | −12.136 | 1.00 | 0.00 | N |
| ATOM | 3420 | CA | ALA | A 439 | 58.074 | 65.737 | −11.311 | 1.00 | 0.00 | C |
| ATOM | 3421 | C | ALA | A 439 | 58.733 | 64.462 | −10.786 | 1.00 | 0.00 | C |
| ATOM | 3422 | O | ALA | A 439 | 59.954 | 64.318 | −10.837 | 1.00 | 0.00 | O |
| ATOM | 3423 | CB | ALA | A 439 | 57.660 | 66.644 | −10.143 | 1.00 | 0.00 | C |
| ATOM | 3424 | N | GLU | A 440 | 57.925 | 63.538 | −10.274 | 1.00 | 0.00 | N |
| ATOM | 3425 | CA | GLU | A 440 | 58.468 | 62.282 | −9.761 | 1.00 | 0.00 | C |
| ATOM | 3426 | C | GLU | A 440 | 59.121 | 61.460 | −10.875 | 1.00 | 0.00 | C |
| ATOM | 3427 | O | GLU | A 440 | 60.177 | 60.855 | −10.676 | 1.00 | 0.00 | O |
| ATOM | 3428 | CB | GLU | A 440 | 57.366 | 61.449 | −9.094 | 1.00 | 0.00 | C |
| ATOM | 3429 | CG | GLU | A 440 | 56.798 | 62.084 | −7.836 | 1.00 | 0.00 | C |
| ATOM | 3430 | CD | GLU | A 440 | 56.139 | 61.070 | −6.918 | 1.00 | 0.00 | C |
| ATOM | 3431 | OE1 | GLU | A 440 | 55.013 | 60.619 | −7.215 | 1.00 | 0.00 | O |
| ATOM | 3432 | OE2 | GLU | A 440 | 56.765 | 60.712 | −5.903 | 1.00 | 0.00 | O |
| ATOM | 3433 | N | MET | A 441 | 58.497 | 61.433 | −12.048 | 1.00 | 0.00 | N |
| ATOM | 3434 | CA | MET | A 441 | 59.050 | 60.662 | −13.160 | 1.00 | 0.00 | C |
| ATOM | 3435 | C | MET | A 441 | 60.327 | 61.279 | −13.723 | 1.00 | 0.00 | C |
| ATOM | 3436 | O | MET | A 441 | 61.326 | 60.583 | −13.915 | 1.00 | 0.00 | O |
| ATOM | 3437 | CB | MET | A 441 | 58.013 | 60.508 | −14.275 | 1.00 | 0.00 | C |
| ATOM | 3438 | CG | MET | A 441 | 58.521 | 59.759 | −15.507 | 1.00 | 0.00 | C |
| ATOM | 3439 | SD | MET | A 441 | 57.249 | 59.561 | −16.783 | 1.00 | 0.00 | S |
| ATOM | 3440 | CE | MET | A 441 | 56.178 | 58.340 | −16.009 | 1.00 | 0.00 | C |
| ATOM | 3441 | N | LEU | A 442 | 60.305 | 62.583 | −13.984 | 1.00 | 0.00 | N |
| ATOM | 3442 | CA | LEU | A 442 | 61.483 | 63.250 | −14.531 | 1.00 | 0.00 | C |
| ATOM | 3443 | C | LEU | A 442 | 62.698 | 63.167 | −13.620 | 1.00 | 0.00 | C |
| ATOM | 3444 | O | LEU | A 442 | 63.827 | 63.032 | −14.095 | 1.00 | 0.00 | O |
| ATOM | 3445 | CB | LEU | A 442 | 61.173 | 64.718 | −14.844 | 1.00 | 0.00 | C |
| ATOM | 3446 | CG | LEU | A 442 | 60.550 | 64.989 | −16.219 | 1.00 | 0.00 | C |
| ATOM | 3447 | CD1 | LEU | A 442 | 59.972 | 66.398 | −16.256 | 1.00 | 0.00 | C |
| ATOM | 3448 | CD2 | LEU | A 442 | 61.598 | 64.811 | −17.305 | 1.00 | 0.00 | C |
| ATOM | 3449 | N | SER | A 443 | 62.477 | 63.228 | −12.310 | 1.00 | 0.00 | N |
| ATOM | 3450 | CA | SER | A 443 | 63.594 | 63.173 | −11.377 | 1.00 | 0.00 | C |
| ATOM | 3451 | C | SER | A 443 | 64.002 | 61.750 | −10.998 | 1.00 | 0.00 | C |
| ATOM | 3452 | O | SER | A 443 | 65.058 | 61.546 | −10.393 | 1.00 | 0.00 | O |
| ATOM | 3453 | CB | SER | A 443 | 63.263 | 63.971 | −10.110 | 1.00 | 0.00 | C |
| ATOM | 3454 | OG | SER | A 443 | 62.172 | 63.400 | −9.410 | 1.00 | 0.00 | O |
| ATOM | 3455 | N | ALA | A 444 | 63.178 | 60.770 | −11.364 | 1.00 | 0.00 | N |
| ATOM | 3456 | CA | ALA | A 444 | 63.458 | 59.370 | −11.041 | 1.00 | 0.00 | C |
| ATOM | 3457 | C | ALA | A 444 | 64.616 | 58.781 | −11.845 | 1.00 | 0.00 | C |
| ATOM | 3458 | O | ALA | A 444 | 65.262 | 57.832 | −11.403 | 1.00 | 0.00 | O |
| ATOM | 3459 | CB | ALA | A 444 | 62.203 | 58.522 | −11.254 | 1.00 | 0.00 | C |
| ATOM | 3460 | N | TRP | A 445 | 64.878 | 59.345 | −13.020 | 1.00 | 0.00 | N |
| ATOM | 3461 | CA | TRP | A 445 | 65.947 | 58.844 | −13.881 | 1.00 | 0.00 | C |
| ATOM | 3462 | C | TRP | A 445 | 67.332 | 58.915 | −13.252 | 1.00 | 0.00 | C |
| ATOM | 3463 | O | TRP | A 445 | 68.198 | 58.093 | −13.560 | 1.00 | 0.00 | O |
| ATOM | 3464 | CB | TRP | A 445 | 65.943 | 59.593 | −15.218 | 1.00 | 0.00 | C |
| ATOM | 3465 | CG | TRP | A 445 | 64.664 | 59.428 | −15.968 | 1.00 | 0.00 | C |
| ATOM | 3466 | CD1 | TRP | A 445 | 63.670 | 60.354 | −16.109 | 1.00 | 0.00 | C |
| ATOM | 3467 | CD2 | TRP | A 445 | 64.213 | 58.248 | −16.644 | 1.00 | 0.00 | C |
| ATOM | 3468 | NE1 | TRP | A 445 | 62.628 | 59.823 | −16.829 | 1.00 | 0.00 | N |
| ATOM | 3469 | CE2 | TRP | A 445 | 62.934 | 58.532 | −17.170 | 1.00 | 0.00 | C |
| ATOM | 3470 | CE3 | TRP | A 445 | 64.766 | 56.976 | −16.856 | 1.00 | 0.00 | C |
| ATOM | 3471 | CZ2 | TRP | A 445 | 62.193 | 57.588 | −17.898 | 1.00 | 0.00 | C |
| ATOM | 3472 | CZ3 | TRP | A 445 | 64.030 | 56.036 | −17.578 | 1.00 | 0.00 | C |
| ATOM | 3473 | CH2 | TRP | A 445 | 62.755 | 56.351 | −18.091 | 1.00 | 0.00 | C |
| ATOM | 3474 | N | HIS | A 446 | 67.545 | 59.895 | −12.379 | 1.00 | 0.00 | N |
| ATOM | 3475 | CA | HIS | A 446 | 68.834 | 60.044 | −11.714 | 1.00 | 0.00 | C |
| ATOM | 3476 | C | HIS | A 446 | 68.713 | 59.940 | −10.206 | 1.00 | 0.00 | C |
| ATOM | 3477 | O | HIS | A 446 | 67.626 | 60.069 | −9.640 | 1.00 | 0.00 | O |
| ATOM | 3478 | CB | HIS | A 446 | 69.467 | 61.411 | −11.997 | 1.00 | 0.00 | C |
| ATOM | 3479 | CG | HIS | A 446 | 69.879 | 61.623 | −13.418 | 1.00 | 0.00 | C |
| ATOM | 3480 | ND1 | HIS | A 446 | 69.050 | 62.200 | −14.354 | 1.00 | 0.00 | N |
| ATOM | 3481 | CD2 | HIS | A 446 | 71.048 | 61.370 | −14.053 | 1.00 | 0.00 | C |
| ATOM | 3482 | CE1 | HIS | A 446 | 69.691 | 62.297 | −15.505 | 1.00 | 0.00 | C |
| ATOM | 3483 | NE2 | HIS | A 446 | 70.905 | 61.801 | −15.350 | 1.00 | 0.00 | N |
| ATOM | 3484 | N | SER | A 447 | 69.856 | 59.712 | −9.568 | 1.00 | 0.00 | N |
| ATOM | 3485 | CA | SER | A 447 | 69.944 | 59.672 | −8.122 | 1.00 | 0.00 | C |
| ATOM | 3486 | C | SER | A 447 | 70.427 | 61.099 | −7.855 | 1.00 | 0.00 | C |
| ATOM | 3487 | O | SER | A 447 | 71.319 | 61.587 | −8.551 | 1.00 | 0.00 | O |
| ATOM | 3488 | CB | SER | A 447 | 70.993 | 58.649 | −7.680 | 1.00 | 0.00 | C |
| ATOM | 3489 | OG | SER | A 447 | 71.002 | 58.509 | −6.273 | 1.00 | 0.00 | O |
| ATOM | 3490 | N | TRP | A 448 | 69.834 | 61.784 | −6.883 | 1.00 | 0.00 | N |
| ATOM | 3491 | CA | TRP | A 448 | 70.231 | 63.161 | −6.617 | 1.00 | 0.00 | C |
| ATOM | 3492 | C | TRP | A 448 | 70.916 | 63.388 | −5.281 | 1.00 | 0.00 | C |
| ATOM | 3493 | O | TRP | A 448 | 70.587 | 62.751 | −4.284 | 1.00 | 0.00 | O |
| ATOM | 3494 | CB | TRP | A 448 | 69.017 | 64.090 | −6.706 | 1.00 | 0.00 | C |
| ATOM | 3495 | CG | TRP | A 448 | 68.374 | 64.123 | −8.059 | 1.00 | 0.00 | C |
| ATOM | 3496 | CD1 | TRP | A 448 | 67.544 | 63.184 | −8.594 | 1.00 | 0.00 | C |
| ATOM | 3497 | CD2 | TRP | A 448 | 68.520 | 65.144 | −9.052 | 1.00 | 0.00 | C |
| ATOM | 3498 | NE1 | TRP | A 448 | 67.163 | 63.554 | −9.860 | 1.00 | 0.00 | N |
| ATOM | 3499 | CE2 | TRP | A 448 | 67.747 | 64.754 | −10.167 | 1.00 | 0.00 | C |
| ATOM | 3500 | CE3 | TRP | A 448 | 69.232 | 66.353 | −9.108 | 1.00 | 0.00 | C |
| ATOM | 3501 | CZ2 | TRP | A 448 | 67.664 | 65.528 | −11.327 | 1.00 | 0.00 | C |
| ATOM | 3502 | CZ3 | TRP | A 448 | 69.149 | 67.124 | −10.262 | 1.00 | 0.00 | C |
| ATOM | 3503 | CH2 | TRP | A 448 | 68.369 | 66.706 | −11.358 | 1.00 | 0.00 | C |
| ATOM | 3504 | N | ASP | A 449 | 71.873 | 64.312 | −5.277 | 1.00 | 0.00 | N |
| ATOM | 3505 | CA | ASP | A 449 | 72.598 | 64.661 | −4.062 | 1.00 | 0.00 | C |
| ATOM | 3506 | C | ASP | A 449 | 71.600 | 65.348 | −3.135 | 1.00 | 0.00 | C |
| ATOM | 3507 | O | ASP | A 449 | 70.718 | 66.072 | −3.594 | 1.00 | 0.00 | O |
| ATOM | 3508 | CB | ASP | A 449 | 73.754 | 65.610 | −4.393 | 1.00 | 0.00 | C |
| ATOM | 3509 | CG | ASP | A 449 | 74.627 | 65.915 | −3.187 | 1.00 | 0.00 | C |
| ATOM | 3510 | OD1 | ASP | A 449 | 74.188 | 66.678 | −2.297 | 1.00 | 0.00 | O |
| ATOM | 3511 | OD2 | ASP | A 449 | 75.756 | 65.381 | −3.128 | 1.00 | 0.00 | O |
| ATOM | 3512 | N | GLY | A 450 | 71.737 | 65.113 | −1.835 | 1.00 | 0.00 | N |
| ATOM | 3513 | CA | GLY | A 450 | 70.828 | 65.713 | −0.876 | 1.00 | 0.00 | C |
| ATOM | 3514 | C | GLY | A 450 | 70.704 | 67.221 | −0.991 | 1.00 | 0.00 | C |
| ATOM | 3515 | O | GLY | A 450 | 69.661 | 67.788 | −0.664 | 1.00 | 0.00 | O |
| ATOM | 3516 | N | MET | A 451 | 71.764 | 67.875 | −1.454 | 1.00 | 0.00 | N |
| ATOM | 3517 | CA | MET | A 451 | 71.752 | 69.327 | −1.593 | 1.00 | 0.00 | C |
| ATOM | 3518 | C | MET | A 451 | 70.770 | 69.822 | −2.650 | 1.00 | 0.00 | C |
| ATOM | 3519 | O | MET | A 451 | 70.391 | 70.992 | −2.647 | 1.00 | 0.00 | O |
| ATOM | 3520 | CB | MET | A 451 | 73.153 | 69.842 | −1.937 | 1.00 | 0.00 | C |
| ATOM | 3521 | CG | MET | A 451 | 74.196 | 69.602 | −0.862 | 1.00 | 0.00 | C |
| ATOM | 3522 | SD | MET | A 451 | 75.755 | 70.439 | −1.246 | 1.00 | 0.00 | S |
| ATOM | 3523 | CE | MET | A 451 | 76.604 | 69.173 | −2.220 | 1.00 | 0.00 | C |
| ATOM | 3524 | N | ALA | A 452 | 70.367 | 68.937 | −3.557 | 1.00 | 0.00 | N |
| ATOM | 3525 | CA | ALA | A 452 | 69.436 | 69.308 | −4.618 | 1.00 | 0.00 | C |
| ATOM | 3526 | C | ALA | A 452 | 68.020 | 69.480 | −4.080 | 1.00 | 0.00 | C |
| ATOM | 3527 | O | ALA | A 452 | 67.151 | 70.027 | −4.762 | 1.00 | 0.00 | O |
| ATOM | 3528 | CB | ALA | A 452 | 69.451 | 68.253 | −5.719 | 1.00 | 0.00 | C |
| ATOM | 3529 | N | ARG | A 453 | 67.797 | 69.002 | −2.859 | 1.00 | 0.00 | N |
| ATOM | 3530 | CA | ARG | A 453 | 66.490 | 69.099 | −2.211 | 1.00 | 0.00 | C |
| ATOM | 3531 | C | ARG | A 453 | 65.363 | 68.513 | −3.062 | 1.00 | 0.00 | C |
| ATOM | 3532 | O | ARG | A 453 | 64.230 | 68.994 | −3.023 | 1.00 | 0.00 | O |
| ATOM | 3533 | CB | ARG | A 453 | 66.186 | 70.564 | −1.878 | 1.00 | 0.00 | C |
| ATOM | 3534 | CG | ARG | A 453 | 67.256 | 71.230 | −1.023 | 1.00 | 0.00 | C |
| ATOM | 3535 | CD | ARG | A 453 | 66.948 | 72.701 | −0.782 | 1.00 | 0.00 | C |
| ATOM | 3536 | NE | ARG | A 453 | 65.729 | 72.891 | −0.001 | 1.00 | 0.00 | N |
| ATOM | 3537 | CZ | ARG | A 453 | 65.185 | 74.075 | 0.262 | 1.00 | 0.00 | C |
| ATOM | 3538 | NH1 | ARG | A 453 | 65.752 | 75.186 | −0.196 | 1.00 | 0.00 | N |
| ATOM | 3539 | NH2 | ARG | A 453 | 64.074 | 74.150 | 0.985 | 1.00 | 0.00 | N |
| ATOM | 3540 | N | ILE | A 454 | 65.674 | 67.471 | −3.825 | 1.00 | 0.00 | N |
| ATOM | 3541 | CA | ILE | A 454 | 64.682 | 66.828 | −4.679 | 1.00 | 0.00 | C |
| ATOM | 3542 | C | ILE | A 454 | 63.639 | 66.076 | −3.851 | 1.00 | 0.00 | C |
| ATOM | 3543 | O | ILE | A 454 | 62.439 | 66.311 | −3.999 | 1.00 | 0.00 | O |
| ATOM | 3544 | CB | ILE | A 454 | 65.351 | 65.845 | −5.665 | 1.00 | 0.00 | C |
| ATOM | 3545 | CG1 | ILE | A 454 | 66.318 | 66.606 | −6.583 | 1.00 | 0.00 | C |
| ATOM | 3546 | CG2 | ILE | A 454 | 64.288 | 65.109 | −6.477 | 1.00 | 0.00 | C |
| ATOM | 3547 | CD1 | ILE | A 454 | 65.670 | 67.696 | −7.424 | 1.00 | 0.00 | C |
| ATOM | 3548 | N | GLU | A 455 | 64.095 | 65.176 | −2.983 | 1.00 | 0.00 | N |
| ATOM | 3549 | CA | GLU | A 455 | 63.178 | 64.407 | −2.143 | 1.00 | 0.00 | C |
| ATOM | 3550 | C | GLU | A 455 | 62.324 | 65.346 | −1.298 | 1.00 | 0.00 | C |
| ATOM | 3551 | O | GLU | A 455 | 61.130 | 65.114 | −1.102 | 1.00 | 0.00 | O |
| ATOM | 3552 | CB | GLU | A 455 | 63.945 | 63.453 | −1.215 | 1.00 | 0.00 | C |
| ATOM | 3553 | CG | GLU | A 455 | 64.533 | 62.220 | −1.890 | 1.00 | 0.00 | C |
| ATOM | 3554 | CD | GLU | A 455 | 65.881 | 62.476 | −2.542 | 1.00 | 0.00 | C |
| ATOM | 3555 | OE1 | GLU | A 455 | 66.349 | 63.636 | −2.525 | 1.00 | 0.00 | O |
| ATOM | 3556 | OE2 | GLU | A 455 | 66.472 | 61.509 | −3.072 | 1.00 | 0.00 | O |
| ATOM | 3557 | N | GLU | A 456 | 62.948 | 66.408 | −0.799 | 1.00 | 0.00 | N |
| ATOM | 3558 | CA | GLU | A 456 | 62.257 | 67.390 | 0.026 | 1.00 | 0.00 | C |
| ATOM | 3559 | C | GLU | A 456 | 61.070 | 68.007 | −0.712 | 1.00 | 0.00 | C |
| ATOM | 3560 | O | GLU | A 456 | 59.938 | 67.988 | −0.222 | 1.00 | 0.00 | O |
| ATOM | 3561 | CB | GLU | A 456 | 63.227 | 68.499 | 0.435 | 1.00 | 0.00 | C |
| ATOM | 3562 | CG | GLU | A 456 | 62.661 | 69.474 | 1.449 | 1.00 | 0.00 | C |
| ATOM | 3563 | CD | GLU | A 456 | 63.537 | 70.699 | 1.626 | 1.00 | 0.00 | C |
| ATOM | 3564 | OE1 | GLU | A 456 | 64.769 | 70.575 | 1.481 | 1.00 | 0.00 | O |
| ATOM | 3565 | OE2 | GLU | A 456 | 62.995 | 71.785 | 1.922 | 1.00 | 0.00 | O |
| ATOM | 3566 | N | ARG | A 457 | 61.333 | 68.560 | −1.892 | 1.00 | 0.00 | N |
| ATOM | 3567 | CA | ARG | A 457 | 60.282 | 69.192 | −2.685 | 1.00 | 0.00 | C |
| ATOM | 3568 | C | ARG | A 457 | 59.187 | 68.212 | −3.109 | 1.00 | 0.00 | C |
| ATOM | 3569 | O | ARG | A 457 | 58.004 | 68.555 | −3.101 | 1.00 | 0.00 | O |
| ATOM | 3570 | CB | ARG | A 457 | 60.895 | 69.875 | −3.917 | 1.00 | 0.00 | C |
| ATOM | 3571 | CG | ARG | A 457 | 61.309 | 71.336 | −3.699 | 1.00 | 0.00 | C |
| ATOM | 3572 | CD | ARG | A 457 | 62.238 | 71.517 | −2.498 | 1.00 | 0.00 | C |
| ATOM | 3573 | NE | ARG | A 457 | 62.497 | 72.929 | −2.200 | 1.00 | 0.00 | N |
| ATOM | 3574 | CZ | ARG | A 457 | 63.347 | 73.705 | −2.870 | 1.00 | 0.00 | C |
| ATOM | 3575 | NH1 | ARG | A 457 | 64.041 | 73.217 | −3.892 | 1.00 | 0.00 | N |
| ATOM | 3576 | NH2 | ARG | A 457 | 63.504 | 74.977 | −2.517 | 1.00 | 0.00 | N |
| ATOM | 3577 | N | LEU | A 458 | 59.573 | 66.992 | −3.471 | 1.00 | 0.00 | N |
| ATOM | 3578 | CA | LEU | A 458 | 58.590 | 65.997 | −3.888 | 1.00 | 0.00 | C |
| ATOM | 3579 | C | LEU | A 458 | 57.697 | 65.554 | −2.730 | 1.00 | 0.00 | C |
| ATOM | 3580 | O | LEU | A 458 | 56.508 | 65.309 | −2.921 | 1.00 | 0.00 | O |
| ATOM | 3581 | CB | LEU | A 458 | 59.284 | 64.783 | −4.509 | 1.00 | 0.00 | C |
| ATOM | 3582 | CG | LEU | A 458 | 60.000 | 65.079 | −5.836 | 1.00 | 0.00 | C |
| ATOM | 3583 | CD1 | LEU | A 458 | 60.686 | 63.816 | −6.345 | 1.00 | 0.00 | C |
| ATOM | 3584 | CD2 | LEU | A 458 | 58.986 | 65.597 | −6.865 | 1.00 | 0.00 | C |
| ATOM | 3585 | N | GLU | A 459 | 58.263 | 65.452 | −1.531 | 1.00 | 0.00 | N |
| ATOM | 3586 | CA | GLU | A 459 | 57.471 | 65.050 | −0.375 | 1.00 | 0.00 | C |
| ATOM | 3587 | C | GLU | A 459 | 56.427 | 66.128 | −0.094 | 1.00 | 0.00 | C |
| ATOM | 3588 | O | GLU | A 459 | 55.260 | 65.827 | 0.163 | 1.00 | 0.00 | O |
| ATOM | 3589 | CB | GLU | A 459 | 58.364 | 64.842 | 0.855 | 1.00 | 0.00 | C |
| ATOM | 3590 | CG | GLU | A 459 | 57.595 | 64.349 | 2.085 | 1.00 | 0.00 | C |
| ATOM | 3591 | CD | GLU | A 459 | 58.499 | 63.761 | 3.158 | 1.00 | 0.00 | C |
| ATOM | 3592 | OE1 | GLU | A 459 | 59.213 | 64.532 | 3.828 | 1.00 | 0.00 | O |
| ATOM | 3593 | OE2 | GLU | A 459 | 58.500 | 62.522 | 3.323 | 1.00 | 0.00 | O |
| ATOM | 3594 | N | GLN | A 460 | 56.846 | 67.388 | −0.153 | 1.00 | 0.00 | N |
| ATOM | 3595 | CA | GLN | A 460 | 55.924 | 68.493 | 0.079 | 1.00 | 0.00 | C |
| ATOM | 3596 | C | GLN | A 460 | 54.787 | 68.432 | −0.949 | 1.00 | 0.00 | C |
| ATOM | 3597 | O | GLN | A 460 | 53.612 | 68.514 | −0.598 | 1.00 | 0.00 | O |
| ATOM | 3598 | CB | GLN | A 460 | 56.660 | 69.829 | −0.045 | 1.00 | 0.00 | C |
| ATOM | 3599 | CG | GLN | A 460 | 55.777 | 71.040 | 0.210 | 1.00 | 0.00 | C |
| ATOM | 3600 | CD | GLN | A 460 | 56.444 | 72.346 | −0.167 | 1.00 | 0.00 | C |
| ATOM | 3601 | OE1 | GLN | A 460 | 55.924 | 73.423 | 0.127 | 1.00 | 0.00 | O |
| ATOM | 3602 | NE2 | GLN | A 460 | 57.592 | 72.261 | −0.830 | 1.00 | 0.00 | N |
| ATOM | 3603 | N | ALA | A 461 | 55.141 | 68.279 | −2.222 | 1.00 | 0.00 | N |
| ATOM | 3604 | CA | ALA | A 461 | 54.130 | 68.220 | −3.273 | 1.00 | 0.00 | C |
| ATOM | 3605 | C | ALA | A 461 | 53.140 | 67.071 | −3.069 | 1.00 | 0.00 | C |
| ATOM | 3606 | O | ALA | A 461 | 51.929 | 67.271 | −3.161 | 1.00 | 0.00 | O |
| ATOM | 3607 | CB | ALA | A 461 | 54.802 | 68.103 | −4.645 | 1.00 | 0.00 | C |
| ATOM | 3608 | N | ARG | A 462 | 53.653 | 65.872 | −2.800 | 1.00 | 0.00 | N |
| ATOM | 3609 | CA | ARG | A 462 | 52.793 | 64.709 | −2.589 | 1.00 | 0.00 | C |
| ATOM | 3610 | C | ARG | A 462 | 51.856 | 64.925 | −1.402 | 1.00 | 0.00 | C |
| ATOM | 3611 | O | ARG | A 462 | 50.674 | 64.585 | −1.453 | 1.00 | 0.00 | O |
| ATOM | 3612 | CB | ARG | A 462 | 53.628 | 63.444 | −2.332 | 1.00 | 0.00 | C |
| ATOM | 3613 | CG | ARG | A 462 | 54.381 | 62.881 | −3.541 | 1.00 | 0.00 | C |
| ATOM | 3614 | CD | ARG | A 462 | 54.829 | 61.444 | −3.251 | 1.00 | 0.00 | C |
| ATOM | 3615 | NE | ARG | A 462 | 55.740 | 61.374 | −2.109 | 1.00 | 0.00 | N |
| ATOM | 3616 | CZ | ARG | A 462 | 57.059 | 61.524 | −2.195 | 1.00 | 0.00 | C |
| ATOM | 3617 | NH1 | ARG | A 462 | 57.631 | 61.746 | −3.374 | 1.00 | 0.00 | N |
| ATOM | 3618 | NH2 | ARG | A 462 | 57.810 | 61.459 | −1.102 | 1.00 | 0.00 | N |
| ATOM | 3619 | N | ARG | A 463 | 52.389 | 65.494 | −0.327 | 1.00 | 0.00 | N |
| ATOM | 3620 | CA | ARG | A 463 | 51.584 | 65.717 | 0.863 | 1.00 | 0.00 | C |
| ATOM | 3621 | C | ARG | A 463 | 50.499 | 66.784 | 0.729 | 1.00 | 0.00 | C |
| ATOM | 3622 | O | ARG | A 463 | 49.402 | 66.609 | 1.255 | 1.00 | 0.00 | O |
| ATOM | 3623 | CB | ARG | A 463 | 52.503 | 66.000 | 2.054 | 1.00 | 0.00 | C |
| ATOM | 3624 | CG | ARG | A 463 | 53.280 | 64.748 | 2.436 | 1.00 | 0.00 | C |
| ATOM | 3625 | CD | ARG | A 463 | 54.193 | 64.919 | 3.631 | 1.00 | 0.00 | C |
| ATOM | 3626 | NE | ARG | A 463 | 54.722 | 63.619 | 4.032 | 1.00 | 0.00 | N |
| ATOM | 3627 | CZ | ARG | A 463 | 55.484 | 63.411 | 5.100 | 1.00 | 0.00 | C |
| ATOM | 3628 | NH1 | ARG | A 463 | 55.817 | 64.426 | 5.885 | 1.00 | 0.00 | N |
| ATOM | 3629 | NH2 | ARG | A 463 | 55.900 | 62.184 | 5.388 | 1.00 | 0.00 | N |
| ATOM | 3630 | N | GLU | A 464 | 50.780 | 67.879 | 0.025 | 1.00 | 0.00 | N |
| ATOM | 3631 | CA | GLU | A 464 | 49.760 | 68.912 | −0.139 | 1.00 | 0.00 | C |
| ATOM | 3632 | C | GLU | A 464 | 48.655 | 68.409 | −1.067 | 1.00 | 0.00 | C |
| ATOM | 3633 | O | GLU | A 464 | 47.483 | 68.718 | −0.861 | 1.00 | 0.00 | O |
| ATOM | 3634 | CB | GLU | A 464 | 50.368 | 70.203 | −0.691 | 1.00 | 0.00 | C |
| ATOM | 3635 | CG | GLU | A 464 | 51.515 | 70.747 | 0.153 | 1.00 | 0.00 | C |
| ATOM | 3636 | CD | GLU | A 464 | 51.087 | 71.173 | 1.552 | 1.00 | 0.00 | C |
| ATOM | 3637 | OE1 | GLU | A 464 | 50.050 | 70.691 | 2.054 | 1.00 | 0.00 | O |
| ATOM | 3638 | OE2 | GLU | A 464 | 51.804 | 71.989 | 2.163 | 1.00 | 0.00 | O |
| ATOM | 3639 | N | LEU | A 465 | 49.022 | 67.644 | −2.092 | 1.00 | 0.00 | N |
| ATOM | 3640 | CA | LEU | A 465 | 48.016 | 67.098 | −3.003 | 1.00 | 0.00 | C |
| ATOM | 3641 | C | LEU | A 465 | 47.212 | 66.032 | −2.262 | 1.00 | 0.00 | C |
| ATOM | 3642 | O | LEU | A 465 | 45.991 | 65.956 | −2.398 | 1.00 | 0.00 | O |
| ATOM | 3643 | CB | LEU | A 465 | 48.669 | 66.473 | −4.244 | 1.00 | 0.00 | C |
| ATOM | 3644 | CG | LEU | A 465 | 47.699 | 65.779 | −5.215 | 1.00 | 0.00 | C |
| ATOM | 3645 | CD1 | LEU | A 465 | 46.661 | 66.785 | −5.708 | 1.00 | 0.00 | C |
| ATOM | 3646 | CD2 | LEU | A 465 | 48.464 | 65.186 | −6.393 | 1.00 | 0.00 | C |
| ATOM | 3647 | N | SER | A 466 | 47.902 | 65.214 | −1.468 | 1.00 | 0.00 | N |
| ATOM | 3648 | CA | SER | A 466 | 47.234 | 64.157 | −0.712 | 1.00 | 0.00 | C |
| ATOM | 3649 | C | SER | A 466 | 46.247 | 64.759 | 0.277 | 1.00 | 0.00 | C |
| ATOM | 3650 | O | SER | A 466 | 45.140 | 64.247 | 0.459 | 1.00 | 0.00 | O |
| ATOM | 3651 | CB | SER | A 466 | 48.257 | 63.301 | 0.043 | 1.00 | 0.00 | C |
| ATOM | 3652 | OG | SER | A 466 | 49.052 | 62.542 | −0.854 | 1.00 | 0.00 | O |
| ATOM | 3653 | N | LEU | A 467 | 46.653 | 65.852 | 0.914 | 1.00 | 0.00 | N |
| ATOM | 3654 | CA | LEU | A 467 | 45.791 | 66.512 | 1.883 | 1.00 | 0.00 | C |
| ATOM | 3655 | C | LEU | A 467 | 44.478 | 66.949 | 1.238 | 1.00 | 0.00 | C |
| ATOM | 3656 | O | LEU | A 467 | 43.402 | 66.757 | 1.810 | 1.00 | 0.00 | O |
| ATOM | 3657 | CB | LEU | A 467 | 46.500 | 67.735 | 2.477 | 1.00 | 0.00 | C |
| ATOM | 3658 | CG | LEU | A 467 | 45.768 | 68.414 | 3.635 | 1.00 | 0.00 | C |
| ATOM | 3659 | CD1 | LEU | A 467 | 45.862 | 67.528 | 4.870 | 1.00 | 0.00 | C |
| ATOM | 3660 | CD2 | LEU | A 467 | 46.382 | 69.786 | 3.914 | 1.00 | 0.00 | C |
| ATOM | 3661 | N | PHE | A 468 | 44.570 | 67.521 | 0.040 | 1.00 | 0.00 | N |
| ATOM | 3662 | CA | PHE | A 468 | 43.389 | 68.009 | −0.664 | 1.00 | 0.00 | C |
| ATOM | 3663 | C | PHE | A 468 | 42.411 | 66.905 | −1.057 | 1.00 | 0.00 | C |
| ATOM | 3664 | O | PHE | A 468 | 41.270 | 67.190 | −1.406 | 1.00 | 0.00 | O |
| ATOM | 3665 | CB | PHE | A 468 | 43.797 | 68.809 | −1.908 | 1.00 | 0.00 | C |
| ATOM | 3666 | CG | PHE | A 468 | 42.672 | 69.607 | −2.511 | 1.00 | 0.00 | C |
| ATOM | 3667 | CD1 | PHE | A 468 | 41.955 | 70.518 | −1.736 | 1.00 | 0.00 | C |
| ATOM | 3668 | CD2 | PHE | A 468 | 42.318 | 69.443 | −3.847 | 1.00 | 0.00 | C |
| ATOM | 3669 | CE1 | PHE | A 468 | 40.901 | 71.254 | −2.284 | 1.00 | 0.00 | C |
| ATOM | 3670 | CE2 | PHE | A 468 | 41.264 | 70.173 | −4.402 | 1.00 | 0.00 | C |
| ATOM | 3671 | CZ | PHE | A 468 | 40.554 | 71.081 | −3.617 | 1.00 | 0.00 | C |
| ATOM | 3672 | N | GLN | A 469 | 42.849 | 65.648 | −1.003 | 1.00 | 0.00 | N |
| ATOM | 3673 | CA | GLN | A 469 | 41.959 | 64.540 | −1.337 | 1.00 | 0.00 | C |
| ATOM | 3674 | C | GLN | A 469 | 40.929 | 64.328 | −0.229 | 1.00 | 0.00 | C |
| ATOM | 3675 | O | GLN | A 469 | 39.989 | 63.544 | −0.387 | 1.00 | 0.00 | O |
| ATOM | 3676 | CB | GLN | A 469 | 42.753 | 63.248 | −1.548 | 1.00 | 0.00 | C |
| ATOM | 3677 | CG | GLN | A 469 | 43.806 | 63.349 | −2.645 | 1.00 | 0.00 | C |
| ATOM | 3678 | CD | GLN | A 469 | 43.261 | 63.986 | −3.912 | 1.00 | 0.00 | C |
| ATOM | 3679 | OE1 | GLN | A 469 | 43.797 | 64.987 | −4.395 | 1.00 | 0.00 | O |
| ATOM | 3680 | NE2 | GLN | A 469 | 42.193 | 63.414 | −4.453 | 1.00 | 0.00 | N |
| ATOM | 3681 | N | HIS | A 470 | 41.112 | 65.030 | 0.887 | 1.00 | 0.00 | N |
| ATOM | 3682 | CA | HIS | A 470 | 40.198 | 64.941 | 2.029 | 1.00 | 0.00 | C |
| ATOM | 3683 | C | HIS | A 470 | 38.752 | 65.169 | 1.583 | 1.00 | 0.00 | C |
| ATOM | 3684 | O | HIS | A 470 | 38.509 | 65.904 | 0.621 | 1.00 | 0.00 | O |
| ATOM | 3685 | CB | HIS | A 470 | 40.569 | 65.988 | 3.087 | 1.00 | 0.00 | C |
| ATOM | 3686 | CG | HIS | A 470 | 39.589 | 66.071 | 4.217 | 1.00 | 0.00 | C |
| ATOM | 3687 | ND1 | HIS | A 470 | 39.121 | 67.270 | 4.715 | 1.00 | 0.00 | N |
| ATOM | 3688 | CD2 | HIS | A 470 | 38.967 | 65.102 | 4.928 | 1.00 | 0.00 | C |
| ATOM | 3689 | CE1 | HIS | A 470 | 38.251 | 67.033 | 5.681 | 1.00 | 0.00 | C |
| ATOM | 3690 | NE2 | HIS | A 470 | 38.140 | 65.725 | 5.830 | 1.00 | 0.00 | N |
| ATOM | 3691 | N | HIS | A 471 | 37.796 | 64.566 | 2.292 | 1.00 | 0.00 | N |
| ATOM | 3692 | CA | HIS | A 471 | 36.389 | 64.715 | 1.928 | 1.00 | 0.00 | C |
| ATOM | 3693 | C | HIS | A 471 | 35.769 | 66.104 | 2.151 | 1.00 | 0.00 | C |
| ATOM | 3694 | O | HIS | A 471 | 34.551 | 66.274 | 2.017 | 1.00 | 0.00 | O |
| ATOM | 3695 | CB | HIS | A 471 | 35.525 | 63.626 | 2.600 | 1.00 | 0.00 | C |
| ATOM | 3696 | CG | HIS | A 471 | 35.692 | 63.533 | 4.084 | 1.00 | 0.00 | C |
| ATOM | 3697 | ND1 | HIS | A 471 | 36.653 | 62.743 | 4.678 | 1.00 | 0.00 | N |
| ATOM | 3698 | CD2 | HIS | A 471 | 35.010 | 64.121 | 5.096 | 1.00 | 0.00 | C |
| ATOM | 3699 | CE1 | HIS | A 471 | 36.556 | 62.848 | 5.992 | 1.00 | 0.00 | C |
| ATOM | 3700 | NE2 | HIS | A 471 | 35.568 | 63.678 | 6.270 | 1.00 | 0.00 | N |
| ATOM | 3701 | N | ASP | A 472 | 36.603 | 67.085 | 2.505 | 1.00 | 0.00 | N |
| ATOM | 3702 | CA | ASP | A 472 | 36.161 | 68.473 | 2.654 | 1.00 | 0.00 | C |
| ATOM | 3703 | C | ASP | A 472 | 37.108 | 69.343 | 1.835 | 1.00 | 0.00 | C |
| ATOM | 3704 | O | ASP | A 472 | 37.035 | 70.574 | 1.867 | 1.00 | 0.00 | O |
| ATOM | 3705 | CB | ASP | A 472 | 36.183 | 68.934 | 4.111 | 1.00 | 0.00 | C |
| ATOM | 3706 | CG | ASP | A 472 | 35.075 | 68.320 | 4.922 | 1.00 | 0.00 | C |
| ATOM | 3707 | OD1 | ASP | A 472 | 33.912 | 68.411 | 4.480 | 1.00 | 0.00 | O |
| ATOM | 3708 | OD2 | ASP | A 472 | 35.363 | 67.755 | 5.993 | 1.00 | 0.00 | O |
| ATOM | 3709 | N | GLY | A 473 | 38.002 | 68.685 | 1.104 | 1.00 | 0.00 | N |
| ATOM | 3710 | CA | GLY | A 473 | 38.952 | 69.400 | 0.275 | 1.00 | 0.00 | C |
| ATOM | 3711 | C | GLY | A 473 | 38.444 | 69.513 | −1.147 | 1.00 | 0.00 | C |
| ATOM | 3712 | O | GLY | A 473 | 37.751 | 70.468 | −1.490 | 1.00 | 0.00 | O |
| ATOM | 3713 | N | ILE | A 474 | 38.779 | 68.526 | −1.971 | 1.00 | 0.00 | N |
| ATOM | 3714 | CA | ILE | A 474 | 38.367 | 68.511 | −3.370 | 1.00 | 0.00 | C |
| ATOM | 3715 | C | ILE | A 474 | 36.844 | 68.613 | −3.530 | 1.00 | 0.00 | C |
| ATOM | 3716 | O | ILE | A 474 | 36.345 | 69.057 | −4.566 | 1.00 | 0.00 | O |
| ATOM | 3717 | CB | ILE | A 474 | 38.898 | 67.230 | −4.082 | 1.00 | 0.00 | C |
| ATOM | 3718 | CG1 | ILE | A 474 | 38.613 | 67.307 | −5.584 | 1.00 | 0.00 | C |
| ATOM | 3719 | CG2 | ILE | A 474 | 38.277 | 65.980 | −3.457 | 1.00 | 0.00 | C |
| ATOM | 3720 | CD1 | ILE | A 474 | 39.204 | 66.157 | −6.386 | 1.00 | 0.00 | C |
| ATOM | 3721 | N | THR | A 475 | 36.118 | 68.214 | −2.490 | 1.00 | 0.00 | N |
| ATOM | 3722 | CA | THR | A 475 | 34.656 | 68.252 | −2.480 | 1.00 | 0.00 | C |
| ATOM | 3723 | C | THR | A 475 | 34.109 | 69.681 | −2.518 | 1.00 | 0.00 | C |
| ATOM | 3724 | O | THR | A 475 | 32.942 | 69.898 | −2.861 | 1.00 | 0.00 | O |
| ATOM | 3725 | CB | THR | A 475 | 34.102 | 67.611 | −1.205 | 1.00 | 0.00 | C |
| ATOM | 3726 | OG1 | THR | A 475 | 34.637 | 68.306 | −0.072 | 1.00 | 0.00 | O |
| ATOM | 3727 | CG2 | THR | A 475 | 34.484 | 66.130 | −1.124 | 1.00 | 0.00 | C |
| ATOM | 3728 | N | GLY | A 476 | 34.944 | 70.645 | −2.144 | 1.00 | 0.00 | N |
| ATOM | 3729 | CA | GLY | A 476 | 34.506 | 72.030 | −2.124 | 1.00 | 0.00 | C |
| ATOM | 3730 | C | GLY | A 476 | 33.510 | 72.287 | −1.002 | 1.00 | 0.00 | C |
| ATOM | 3731 | O | GLY | A 476 | 32.618 | 73.126 | −1.136 | 1.00 | 0.00 | O |
| ATOM | 3732 | N | THR | A 477 | 33.660 | 71.572 | 0.110 | 1.00 | 0.00 | N |
| ATOM | 3733 | CA | THR | A 477 | 32.750 | 71.731 | 1.237 | 1.00 | 0.00 | C |
| ATOM | 3734 | C | THR | A 477 | 33.371 | 72.333 | 2.499 | 1.00 | 0.00 | C |
| ATOM | 3735 | O | THR | A 477 | 32.853 | 72.135 | 3.598 | 1.00 | 0.00 | O |
| ATOM | 3736 | CB | THR | A 477 | 32.088 | 70.375 | 1.613 | 1.00 | 0.00 | C |
| ATOM | 3737 | OG1 | THR | A 477 | 33.102 | 69.404 | 1.907 | 1.00 | 0.00 | O |
| ATOM | 3738 | CG2 | THR | A 477 | 31.224 | 69.871 | 0.461 | 1.00 | 0.00 | C |
| ATOM | 3739 | N | ALA | A 478 | 34.469 | 73.075 | 2.351 | 1.00 | 0.00 | N |
| ATOM | 3740 | CA | ALA | A 478 | 35.117 | 73.695 | 3.508 | 1.00 | 0.00 | C |
| ATOM | 3741 | C | ALA | A 478 | 34.800 | 75.188 | 3.586 | 1.00 | 0.00 | C |
| ATOM | 3742 | O | ALA | A 478 | 34.330 | 75.780 | 2.617 | 1.00 | 0.00 | O |
| ATOM | 3743 | CB | ALA | A 478 | 36.634 | 73.486 | 3.439 | 1.00 | 0.00 | C |
| ATOM | 3744 | N | LYS | A 479 | 35.049 | 75.805 | 4.737 | 1.00 | 0.00 | N |
| ATOM | 3745 | CA | LYS | A 479 | 34.783 | 77.235 | 4.858 | 1.00 | 0.00 | C |
| ATOM | 3746 | C | LYS | A 479 | 35.761 | 77.985 | 3.960 | 1.00 | 0.00 | C |
| ATOM | 3747 | O | LYS | A 479 | 36.826 | 77.471 | 3.621 | 1.00 | 0.00 | O |
| ATOM | 3748 | CB | LYS | A 479 | 34.923 | 77.698 | 6.314 | 1.00 | 0.00 | C |
| ATOM | 3749 | CG | LYS | A 479 | 33.900 | 77.052 | 7.251 | 1.00 | 0.00 | C |
| ATOM | 3750 | CD | LYS | A 479 | 33.934 | 77.645 | 8.658 | 1.00 | 0.00 | C |
| ATOM | 3751 | CE | LYS | A 479 | 33.032 | 78.863 | 8.800 | 1.00 | 0.00 | C |
| ATOM | 3752 | NZ | LYS | A 479 | 31.571 | 78.531 | 8.775 | 1.00 | 0.00 | N |
| ATOM | 3753 | N | THR | A 480 | 35.391 | 79.204 | 3.589 | 1.00 | 0.00 | N |
| ATOM | 3754 | CA | THR | A 480 | 36.199 | 80.040 | 2.712 | 1.00 | 0.00 | C |
| ATOM | 3755 | C | THR | A 480 | 37.690 | 80.143 | 3.032 | 1.00 | 0.00 | C |
| ATOM | 3756 | O | THR | A 480 | 38.520 | 79.985 | 2.137 | 1.00 | 0.00 | O |
| ATOM | 3757 | CB | THR | A 480 | 35.612 | 81.464 | 2.643 | 1.00 | 0.00 | C |
| ATOM | 3758 | OG1 | THR | A 480 | 34.266 | 81.390 | 2.164 | 1.00 | 0.00 | O |
| ATOM | 3759 | CG2 | THR | A 480 | 36.433 | 82.353 | 1.701 | 1.00 | 0.00 | C |
| ATOM | 3760 | N | HIS | A 481 | 38.042 | 80.400 | 4.290 | 1.00 | 0.00 | N |
| ATOM | 3761 | CA | HIS | A 481 | 39.454 | 80.536 | 4.628 | 1.00 | 0.00 | C |
| ATOM | 3762 | C | HIS | A 481 | 40.207 | 79.209 | 4.586 | 1.00 | 0.00 | C |
| ATOM | 3763 | O | HIS | A 481 | 41.431 | 79.192 | 4.444 | 1.00 | 0.00 | O |
| ATOM | 3764 | CB | HIS | A 481 | 39.635 | 81.226 | 5.993 | 1.00 | 0.00 | C |
| ATOM | 3765 | CG | HIS | A 481 | 39.481 | 80.320 | 7.176 | 1.00 | 0.00 | C |
| ATOM | 3766 | ND1 | HIS | A 481 | 38.258 | 79.860 | 7.614 | 1.00 | 0.00 | N |
| ATOM | 3767 | CD2 | HIS | A 481 | 40.402 | 79.806 | 8.025 | 1.00 | 0.00 | C |
| ATOM | 3768 | CE1 | HIS | A 481 | 38.432 | 79.103 | 8.682 | 1.00 | 0.00 | C |
| ATOM | 3769 | NE2 | HIS | A 481 | 39.724 | 79.053 | 8.952 | 1.00 | 0.00 | N |
| ATOM | 3770 | N | VAL | A 482 | 39.475 | 78.103 | 4.694 | 1.00 | 0.00 | N |
| ATOM | 3771 | CA | VAL | A 482 | 40.090 | 76.774 | 4.640 | 1.00 | 0.00 | C |
| ATOM | 3772 | C | VAL | A 482 | 40.382 | 76.450 | 3.172 | 1.00 | 0.00 | C |
| ATOM | 3773 | O | VAL | A 482 | 41.427 | 75.894 | 2.840 | 1.00 | 0.00 | O |
| ATOM | 3774 | CB | VAL | A 482 | 39.157 | 75.705 | 5.255 | 1.00 | 0.00 | C |
| ATOM | 3775 | CG1 | VAL | A 482 | 39.828 | 74.327 | 5.231 | 1.00 | 0.00 | C |
| ATOM | 3776 | CG2 | VAL | A 482 | 38.819 | 76.096 | 6.693 | 1.00 | 0.00 | C |
| ATOM | 3777 | N | VAL | A 483 | 39.452 | 76.808 | 2.292 | 1.00 | 0.00 | N |
| ATOM | 3778 | CA | VAL | A 483 | 39.645 | 76.592 | 0.862 | 1.00 | 0.00 | C |
| ATOM | 3779 | C | VAL | A 483 | 40.898 | 77.365 | 0.446 | 1.00 | 0.00 | C |
| ATOM | 3780 | O | VAL | A 483 | 41.707 | 76.888 | −0.353 | 1.00 | 0.00 | O |
| ATOM | 3781 | CB | VAL | A 483 | 38.432 | 77.112 | 0.057 | 1.00 | 0.00 | C |
| ATOM | 3782 | CG1 | VAL | A 483 | 38.716 | 77.030 | −1.437 | 1.00 | 0.00 | C |
| ATOM | 3783 | CG2 | VAL | A 483 | 37.191 | 76.285 | 0.411 | 1.00 | 0.00 | C |
| ATOM | 3784 | N | VAL | A 484 | 41.059 | 78.560 | 1.003 | 1.00 | 0.00 | N |
| ATOM | 3785 | CA | VAL | A 484 | 42.224 | 79.379 | 0.698 | 1.00 | 0.00 | C |
| ATOM | 3786 | C | VAL | A 484 | 43.495 | 78.670 | 1.147 | 1.00 | 0.00 | C |
| ATOM | 3787 | O | VAL | A 484 | 44.498 | 78.678 | 0.434 | 1.00 | 0.00 | O |
| ATOM | 3788 | CB | VAL | A 484 | 42.131 | 80.759 | 1.377 | 1.00 | 0.00 | C |
| ATOM | 3789 | CG1 | VAL | A 484 | 43.461 | 81.500 | 1.252 | 1.00 | 0.00 | C |
| ATOM | 3790 | CG2 | VAL | A 484 | 41.018 | 81.568 | 0.730 | 1.00 | 0.00 | C |
| ATOM | 3791 | N | ASP | A 485 | 43.454 | 78.047 | 2.323 | 1.00 | 0.00 | N |
| ATOM | 3792 | CA | ASP | A 485 | 44.621 | 77.326 | 2.822 | 1.00 | 0.00 | C |
| ATOM | 3793 | C | ASP | A 485 | 44.993 | 76.185 | 1.870 | 1.00 | 0.00 | C |
| ATOM | 3794 | O | ASP | A 485 | 46.166 | 75.996 | 1.541 | 1.00 | 0.00 | O |
| ATOM | 3795 | CB | ASP | A 485 | 44.354 | 76.751 | 4.215 | 1.00 | 0.00 | C |
| ATOM | 3796 | CG | ASP | A 485 | 45.600 | 76.156 | 4.843 | 1.00 | 0.00 | C |
| ATOM | 3797 | OD1 | ASP | A 485 | 45.549 | 75.002 | 5.314 | 1.00 | 0.00 | O |
| ATOM | 3798 | OD2 | ASP | A 485 | 46.640 | 76.850 | 4.866 | 1.00 | 0.00 | O |
| ATOM | 3799 | N | TYR | A 486 | 43.996 | 75.418 | 1.434 | 1.00 | 0.00 | N |
| ATOM | 3800 | CA | TYR | A 486 | 44.251 | 74.307 | 0.515 | 1.00 | 0.00 | C |
| ATOM | 3801 | C | TYR | A 486 | 44.872 | 74.832 | −0.777 | 1.00 | 0.00 | C |
| ATOM | 3802 | O | TYR | A 486 | 45.799 | 74.228 | −1.314 | 1.00 | 0.00 | O |
| ATOM | 3803 | CB | TYR | A 486 | 42.951 | 73.571 | 0.174 | 1.00 | 0.00 | C |
| ATOM | 3804 | CG | TYR | A 486 | 42.351 | 72.736 | 1.291 | 1.00 | 0.00 | C |
| ATOM | 3805 | CD1 | TYR | A 486 | 40.971 | 72.715 | 1.492 | 1.00 | 0.00 | C |
| ATOM | 3806 | CD2 | TYR | A 486 | 43.148 | 71.917 | 2.101 | 1.00 | 0.00 | C |
| ATOM | 3807 | CE1 | TYR | A 486 | 40.387 | 71.897 | 2.469 | 1.00 | 0.00 | C |
| ATOM | 3808 | CE2 | TYR | A 486 | 42.569 | 71.090 | 3.086 | 1.00 | 0.00 | C |
| ATOM | 3809 | CZ | TYR | A 486 | 41.186 | 71.090 | 3.257 | 1.00 | 0.00 | C |
| ATOM | 3810 | OH | TYR | A 486 | 40.590 | 70.278 | 4.200 | 1.00 | 0.00 | O |
| ATOM | 3811 | N | GLU | A 487 | 44.357 | 75.958 | −1.270 | 1.00 | 0.00 | N |
| ATOM | 3812 | CA | GLU | A 487 | 44.865 | 76.552 | −2.505 | 1.00 | 0.00 | C |
| ATOM | 3813 | C | GLU | A 487 | 46.320 | 76.979 | −2.341 | 1.00 | 0.00 | C |
| ATOM | 3814 | O | GLU | A 487 | 47.149 | 76.727 | −3.213 | 1.00 | 0.00 | O |
| ATOM | 3815 | CB | GLU | A 487 | 44.030 | 77.772 | −2.920 | 1.00 | 0.00 | C |
| ATOM | 3816 | CG | GLU | A 487 | 44.302 | 78.202 | −4.361 | 1.00 | 0.00 | C |
| ATOM | 3817 | CD | GLU | A 487 | 43.595 | 79.487 | −4.766 | 1.00 | 0.00 | C |
| ATOM | 3818 | OE1 | GLU | A 487 | 42.428 | 79.694 | −4.372 | 1.00 | 0.00 | O |
| ATOM | 3819 | OE2 | GLU | A 487 | 44.210 | 80.286 | −5.502 | 1.00 | 0.00 | O |
| ATOM | 3820 | N | GLN | A 488 | 46.622 | 77.636 | −1.226 | 1.00 | 0.00 | N |
| ATOM | 3821 | CA | GLN | A 488 | 47.986 | 78.084 | −0.957 | 1.00 | 0.00 | C |
| ATOM | 3822 | C | GLN | A 488 | 48.929 | 76.888 | −0.920 | 1.00 | 0.00 | C |
| ATOM | 3823 | O | GLN | A 488 | 50.028 | 76.937 | −1.472 | 1.00 | 0.00 | O |
| ATOM | 3824 | CB | GLN | A 488 | 48.051 | 78.833 | 0.380 | 1.00 | 0.00 | C |
| ATOM | 3825 | CG | GLN | A 488 | 47.360 | 80.189 | 0.363 | 1.00 | 0.00 | C |
| ATOM | 3826 | CD | GLN | A 488 | 47.390 | 80.884 | 1.713 | 1.00 | 0.00 | C |
| ATOM | 3827 | OE1 | GLN | A 488 | 47.028 | 82.054 | 1.825 | 1.00 | 0.00 | O |
| ATOM | 3828 | NE2 | GLN | A 488 | 47.818 | 80.165 | 2.746 | 1.00 | 0.00 | N |
| ATOM | 3829 | N | ARG | A 489 | 48.497 | 75.822 | −0.253 | 1.00 | 0.00 | N |
| ATOM | 3830 | CA | ARG | A 489 | 49.295 | 74.606 | −0.155 | 1.00 | 0.00 | C |
| ATOM | 3831 | C | ARG | A 489 | 49.530 | 74.010 | −1.540 | 1.00 | 0.00 | C |
| ATOM | 3832 | O | ARG | A 489 | 50.643 | 73.593 | −1.867 | 1.00 | 0.00 | O |
| ATOM | 3833 | CB | ARG | A 489 | 48.591 | 73.574 | 0.735 | 1.00 | 0.00 | C |
| ATOM | 3834 | CG | ARG | A 489 | 48.625 | 73.891 | 2.233 | 1.00 | 0.00 | C |
| ATOM | 3835 | CD | ARG | A 489 | 47.696 | 72.954 | 3.006 | 1.00 | 0.00 | C |
| ATOM | 3836 | NE | ARG | A 489 | 47.770 | 73.139 | 4.455 | 1.00 | 0.00 | N |
| ATOM | 3837 | CZ | ARG | A 489 | 48.680 | 72.571 | 5.244 | 1.00 | 0.00 | C |
| ATOM | 3838 | NH1 | ARG | A 489 | 49.606 | 71.771 | 4.730 | 1.00 | 0.00 | N |
| ATOM | 3839 | NH2 | ARG | A 489 | 48.665 | 72.806 | 6.552 | 1.00 | 0.00 | N |
| ATOM | 3840 | N | MET | A 490 | 48.487 | 73.962 | −2.363 | 1.00 | 0.00 | N |
| ATOM | 3841 | CA | MET | A 490 | 48.662 | 73.406 | −3.699 | 1.00 | 0.00 | C |
| ATOM | 3842 | C | MET | A 490 | 49.544 | 74.308 | −4.558 | 1.00 | 0.00 | C |
| ATOM | 3843 | O | MET | A 490 | 50.285 | 73.827 | −5.414 | 1.00 | 0.00 | O |
| ATOM | 3844 | CB | MET | A 490 | 47.305 | 73.159 | −4.373 | 1.00 | 0.00 | C |
| ATOM | 3845 | CG | MET | A 490 | 46.539 | 71.993 | −3.748 | 1.00 | 0.00 | C |
| ATOM | 3846 | SD | MET | A 490 | 45.205 | 71.339 | −4.786 | 1.00 | 0.00 | S |
| ATOM | 3847 | CE | MET | A 490 | 43.887 | 72.488 | −4.381 | 1.00 | 0.00 | C |
| ATOM | 3848 | N | GLN | A 491 | 49.482 | 75.613 | −4.319 | 1.00 | 0.00 | N |
| ATOM | 3849 | CA | GLN | A 491 | 50.303 | 76.543 | −5.084 | 1.00 | 0.00 | C |
| ATOM | 3850 | C | GLN | A 491 | 51.775 | 76.263 | −4.790 | 1.00 | 0.00 | C |
| ATOM | 3851 | O | GLN | A 491 | 52.615 | 76.259 | −5.694 | 1.00 | 0.00 | O |
| ATOM | 3852 | CB | GLN | A 491 | 49.960 | 77.985 | −4.713 | 1.00 | 0.00 | C |
| ATOM | 3853 | CG | GLN | A 491 | 50.638 | 79.023 | −5.585 | 1.00 | 0.00 | C |
| ATOM | 3854 | CD | GLN | A 491 | 50.355 | 78.817 | −7.062 | 1.00 | 0.00 | C |
| ATOM | 3855 | OE1 | GLN | A 491 | 51.044 | 78.050 | −7.741 | 1.00 | 0.00 | O |
| ATOM | 3856 | NE2 | GLN | A 491 | 49.329 | 79.493 | −7.564 | 1.00 | 0.00 | N |
| ATOM | 3857 | N | GLU | A 492 | 52.083 | 76.020 | −3.520 | 1.00 | 0.00 | N |
| ATOM | 3858 | CA | GLU | A 492 | 53.453 | 75.728 | −3.123 | 1.00 | 0.00 | C |
| ATOM | 3859 | C | GLU | A 492 | 53.874 | 74.393 | −3.737 | 1.00 | 0.00 | C |
| ATOM | 3860 | O | GLU | A 492 | 55.019 | 74.223 | −4.154 | 1.00 | 0.00 | O |
| ATOM | 3861 | CB | GLU | A 492 | 53.554 | 75.674 | −1.595 | 1.00 | 0.00 | C |
| ATOM | 3862 | CG | GLU | A 492 | 52.993 | 76.921 | −0.916 | 1.00 | 0.00 | C |
| ATOM | 3863 | CD | GLU | A 492 | 52.973 | 76.826 | 0.599 | 1.00 | 0.00 | C |
| ATOM | 3864 | OE1 | GLU | A 492 | 52.545 | 75.780 | 1.135 | 1.00 | 0.00 | O |
| ATOM | 3865 | OE2 | GLU | A 492 | 53.372 | 77.810 | 1.259 | 1.00 | 0.00 | O |
| ATOM | 3866 | N | ALA | A 493 | 52.939 | 73.448 | −3.798 | 1.00 | 0.00 | N |
| ATOM | 3867 | CA | ALA | A 493 | 53.219 | 72.135 | −4.376 | 1.00 | 0.00 | C |
| ATOM | 3868 | C | ALA | A 493 | 53.553 | 72.289 | −5.861 | 1.00 | 0.00 | C |
| ATOM | 3869 | O | ALA | A 493 | 54.494 | 71.668 | −6.365 | 1.00 | 0.00 | O |
| ATOM | 3870 | CB | ALA | A 493 | 52.010 | 71.212 | −4.199 | 1.00 | 0.00 | C |
| ATOM | 3871 | N | LEU | A 494 | 52.781 | 73.115 | −6.562 | 1.00 | 0.00 | N |
| ATOM | 3872 | CA | LEU | A 494 | 53.024 | 73.336 | −7.986 | 1.00 | 0.00 | C |
| ATOM | 3873 | C | LEU | A 494 | 54.425 | 73.900 | −8.208 | 1.00 | 0.00 | C |
| ATOM | 3874 | O | LEU | A 494 | 55.142 | 73.453 | −9.098 | 1.00 | 0.00 | O |
| ATOM | 3875 | CB | LEU | A 494 | 51.971 | 74.287 | −8.573 | 1.00 | 0.00 | C |
| ATOM | 3876 | CG | LEU | A 494 | 50.574 | 73.683 | −8.752 | 1.00 | 0.00 | C |
| ATOM | 3877 | CD1 | LEU | A 494 | 49.569 | 74.769 | −9.145 | 1.00 | 0.00 | C |
| ATOM | 3878 | CD2 | LEU | A 494 | 50.636 | 72.601 | −9.818 | 1.00 | 0.00 | C |
| ATOM | 3879 | N | LYS | A 495 | 54.812 | 74.872 | −7.385 | 1.00 | 0.00 | N |
| ATOM | 3880 | CA | LYS | A 495 | 56.134 | 75.484 | −7.487 | 1.00 | 0.00 | C |
| ATOM | 3881 | C | LYS | A 495 | 57.221 | 74.450 | −7.200 | 1.00 | 0.00 | C |
| ATOM | 3882 | O | LYS | A 495 | 58.269 | 74.443 | −7.850 | 1.00 | 0.00 | O |
| ATOM | 3883 | CB | LYS | A 495 | 56.253 | 76.660 | −6.510 | 1.00 | 0.00 | C |
| ATOM | 3884 | CG | LYS | A 495 | 55.284 | 77.803 | −6.812 | 1.00 | 0.00 | C |
| ATOM | 3885 | CD | LYS | A 495 | 55.563 | 79.039 | −5.960 | 1.00 | 0.00 | C |
| ATOM | 3886 | CE | LYS | A 495 | 55.364 | 78.767 | −4.477 | 1.00 | 0.00 | C |
| ATOM | 3887 | NZ | LYS | A 495 | 55.650 | 79.976 | −3.647 | 1.00 | 0.00 | N |
| ATOM | 3888 | N | ALA | A 496 | 56.966 | 73.577 | −6.228 | 1.00 | 0.00 | N |
| ATOM | 3889 | CA | ALA | A 496 | 57.917 | 72.528 | −5.881 | 1.00 | 0.00 | C |
| ATOM | 3890 | C | ALA | A 496 | 58.100 | 71.602 | −7.084 | 1.00 | 0.00 | C |
| ATOM | 3891 | O | ALA | A 496 | 59.221 | 71.220 | −7.426 | 1.00 | 0.00 | O |
| ATOM | 3892 | CB | ALA | A 496 | 57.410 | 71.732 | −4.682 | 1.00 | 0.00 | C |
| ATOM | 3893 | N | CYS | A 497 | 56.991 | 71.234 | −7.719 | 1.00 | 0.00 | N |
| ATOM | 3894 | CA | CYS | A 497 | 57.045 | 70.362 | −8.886 | 1.00 | 0.00 | C |
| ATOM | 3895 | C | CYS | A 497 | 57.821 | 71.035 | −10.011 | 1.00 | 0.00 | C |
| ATOM | 3896 | O | CYS | A 497 | 58.659 | 70.405 | −10.653 | 1.00 | 0.00 | O |
| ATOM | 3897 | CB | CYS | A 497 | 55.628 | 70.003 | −9.359 | 1.00 | 0.00 | C |
| ATOM | 3898 | SG | CYS | A 497 | 54.775 | 68.818 | −8.281 | 1.00 | 0.00 | S |
| ATOM | 3899 | N | GLN | A 498 | 57.551 | 72.316 | −10.248 | 1.00 | 0.00 | N |
| ATOM | 3900 | CA | GLN | A 498 | 58.258 | 73.039 | −11.302 | 1.00 | 0.00 | C |
| ATOM | 3901 | C | GLN | A 498 | 59.766 | 73.017 | −11.055 | 1.00 | 0.00 | C |
| ATOM | 3902 | O | GLN | A 498 | 60.545 | 72.760 | −11.971 | 1.00 | 0.00 | O |
| ATOM | 3903 | CB | GLN | A 498 | 57.780 | 74.492 | −11.385 | 1.00 | 0.00 | C |
| ATOM | 3904 | CG | GLN | A 498 | 58.583 | 75.337 | −12.367 | 1.00 | 0.00 | C |
| ATOM | 3905 | CD | GLN | A 498 | 58.076 | 76.764 | −12.475 | 1.00 | 0.00 | C |
| ATOM | 3906 | OE1 | GLN | A 498 | 57.891 | 77.449 | −11.464 | 1.00 | 0.00 | O |
| ATOM | 3907 | NE2 | GLN | A 498 | 57.859 | 77.225 | −13.704 | 1.00 | 0.00 | N |
| ATOM | 3908 | N | MET | A 499 | 60.178 | 73.292 | −9.820 | 1.00 | 0.00 | N |
| ATOM | 3909 | CA | MET | A 499 | 61.603 | 73.297 | −9.484 | 1.00 | 0.00 | C |
| ATOM | 3910 | C | MET | A 499 | 62.252 | 71.947 | −9.800 | 1.00 | 0.00 | C |
| ATOM | 3911 | O | MET | A 499 | 63.304 | 71.887 | −10.435 | 1.00 | 0.00 | O |
| ATOM | 3912 | CB | MET | A 499 | 61.793 | 73.633 | −8.000 | 1.00 | 0.00 | C |
| ATOM | 3913 | CG | MET | A 499 | 63.226 | 73.497 | −7.485 | 1.00 | 0.00 | C |
| ATOM | 3914 | SD | MET | A 499 | 64.443 | 74.506 | −8.376 | 1.00 | 0.00 | S |
| ATOM | 3915 | CE | MET | A 499 | 64.087 | 76.121 | −7.718 | 1.00 | 0.00 | C |
| ATOM | 3916 | N | VAL | A 500 | 61.623 | 70.865 | −9.351 | 1.00 | 0.00 | N |
| ATOM | 3917 | CA | VAL | A 500 | 62.153 | 69.528 | −9.595 | 1.00 | 0.00 | C |
| ATOM | 3918 | C | VAL | A 500 | 62.167 | 69.199 | −11.085 | 1.00 | 0.00 | C |
| ATOM | 3919 | O | VAL | A 500 | 63.152 | 68.682 | −11.609 | 1.00 | 0.00 | O |
| ATOM | 3920 | CB | VAL | A 500 | 61.321 | 68.466 | −8.837 | 1.00 | 0.00 | C |
| ATOM | 3921 | CG1 | VAL | A 500 | 61.744 | 67.065 | −9.250 | 1.00 | 0.00 | C |
| ATOM | 3922 | CG2 | VAL | A 500 | 61.513 | 68.647 | −7.332 | 1.00 | 0.00 | C |
| ATOM | 3923 | N | MET | A 501 | 61.072 | 69.508 | −11.767 | 1.00 | 0.00 | N |
| ATOM | 3924 | CA | MET | A 501 | 60.975 | 69.229 | −13.194 | 1.00 | 0.00 | C |
| ATOM | 3925 | C | MET | A 501 | 62.047 | 69.947 | −14.007 | 1.00 | 0.00 | C |
| ATOM | 3926 | O | MET | A 501 | 62.746 | 69.318 | −14.806 | 1.00 | 0.00 | O |
| ATOM | 3927 | CB | MET | A 501 | 59.585 | 69.612 | −13.705 | 1.00 | 0.00 | C |
| ATOM | 3928 | CG | MET | A 501 | 58.475 | 68.709 | −13.191 | 1.00 | 0.00 | C |
| ATOM | 3929 | SD | MET | A 501 | 56.838 | 69.371 | −13.539 | 1.00 | 0.00 | S |
| ATOM | 3930 | CE | MET | A 501 | 56.676 | 68.974 | −15.250 | 1.00 | 0.00 | C |
| ATOM | 3931 | N | GLN | A 502 | 62.186 | 71.256 | −13.802 | 1.00 | 0.00 | N |
| ATOM | 3932 | CA | GLN | A 502 | 63.171 | 72.028 | −14.555 | 1.00 | 0.00 | C |
| ATOM | 3933 | C | GLN | A 502 | 64.610 | 71.604 | −14.253 | 1.00 | 0.00 | C |
| ATOM | 3934 | O | GLN | A 502 | 65.447 | 71.561 | −15.159 | 1.00 | 0.00 | O |
| ATOM | 3935 | CB | GLN | A 502 | 62.967 | 73.530 | −14.317 | 1.00 | 0.00 | C |
| ATOM | 3936 | CG | GLN | A 502 | 63.208 | 74.016 | −12.900 | 1.00 | 0.00 | C |
| ATOM | 3937 | CD | GLN | A 502 | 64.597 | 74.611 | −12.726 | 1.00 | 0.00 | C |
| ATOM | 3938 | OE1 | GLN | A 502 | 65.348 | 74.753 | −13.697 | 1.00 | 0.00 | O |
| ATOM | 3939 | NE2 | GLN | A 502 | 64.938 | 74.974 | −11.494 | 1.00 | 0.00 | N |
| ATOM | 3940 | N | GLN | A 503 | 64.905 | 71.287 | −12.995 | 1.00 | 0.00 | N |
| ATOM | 3941 | CA | GLN | A 503 | 66.253 | 70.825 | −12.650 | 1.00 | 0.00 | C |
| ATOM | 3942 | C | GLN | A 503 | 66.510 | 69.504 | −13.381 | 1.00 | 0.00 | C |
| ATOM | 3943 | O | GLN | A 503 | 67.595 | 69.277 | −13.914 | 1.00 | 0.00 | O |
| ATOM | 3944 | CB | GLN | A 503 | 66.389 | 70.594 | −11.141 | 1.00 | 0.00 | C |
| ATOM | 3945 | CG | GLN | A 503 | 66.593 | 71.853 | −10.301 | 1.00 | 0.00 | C |
| ATOM | 3946 | CD | GLN | A 503 | 67.940 | 72.511 | −10.549 | 1.00 | 0.00 | C |
| ATOM | 3947 | OE1 | GLN | A 503 | 68.935 | 71.833 | −10.818 | 1.00 | 0.00 | O |
| ATOM | 3948 | NE2 | GLN | A 503 | 67.982 | 73.835 | −10.442 | 1.00 | 0.00 | N |
| ATOM | 3949 | N | SER | A 504 | 65.503 | 68.632 | −13.398 | 1.00 | 0.00 | N |
| ATOM | 3950 | CA | SER | A 504 | 65.625 | 67.338 | −14.067 | 1.00 | 0.00 | C |
| ATOM | 3951 | C | SER | A 504 | 65.855 | 67.486 | −15.567 | 1.00 | 0.00 | C |
| ATOM | 3952 | O | SER | A 504 | 66.715 | 66.812 | −16.138 | 1.00 | 0.00 | O |
| ATOM | 3953 | CB | SER | A 504 | 64.369 | 66.490 | −13.829 | 1.00 | 0.00 | C |
| ATOM | 3954 | OG | SER | A 504 | 64.235 | 66.150 | −12.460 | 1.00 | 0.00 | O |
| ATOM | 3955 | N | VAL | A 505 | 65.087 | 68.361 | −16.206 | 1.00 | 0.00 | N |
| ATOM | 3956 | CA | VAL | A 505 | 65.233 | 68.575 | −17.643 | 1.00 | 0.00 | C |
| ATOM | 3957 | C | VAL | A 505 | 66.647 | 69.040 | −17.974 | 1.00 | 0.00 | C |
| ATOM | 3958 | O | VAL | A 505 | 67.269 | 68.555 | −18.921 | 1.00 | 0.00 | O |
| ATOM | 3959 | CB | VAL | A 505 | 64.224 | 69.623 | −18.165 | 1.00 | 0.00 | C |
| ATOM | 3960 | CG1 | VAL | A 505 | 64.569 | 70.018 | −19.605 | 1.00 | 0.00 | C |
| ATOM | 3961 | CG2 | VAL | A 505 | 62.805 | 69.051 | −18.105 | 1.00 | 0.00 | C |
| ATOM | 3962 | N | TYR | A 506 | 67.160 | 69.977 | −17.187 | 1.00 | 0.00 | N |
| ATOM | 3963 | CA | TYR | A 506 | 68.501 | 70.490 | −17.425 | 1.00 | 0.00 | C |
| ATOM | 3964 | C | TYR | A 506 | 69.557 | 69.378 | −17.364 | 1.00 | 0.00 | C |
| ATOM | 3965 | O | TYR | A 506 | 70.458 | 69.313 | −18.206 | 1.00 | 0.00 | O |
| ATOM | 3966 | CB | TYR | A 506 | 68.827 | 71.584 | −16.407 | 1.00 | 0.00 | C |
| ATOM | 3967 | CG | TYR | A 506 | 70.168 | 72.230 | −16.641 | 1.00 | 0.00 | C |
| ATOM | 3968 | CD1 | TYR | A 506 | 70.475 | 72.804 | −17.875 | 1.00 | 0.00 | C |
| ATOM | 3969 | CD2 | TYR | A 506 | 71.138 | 72.252 | −15.641 | 1.00 | 0.00 | C |
| ATOM | 3970 | CE1 | TYR | A 506 | 71.717 | 73.382 | −18.109 | 1.00 | 0.00 | C |
| ATOM | 3971 | CE2 | TYR | A 506 | 72.388 | 72.826 | −15.868 | 1.00 | 0.00 | C |
| ATOM | 3972 | CZ | TYR | A 506 | 72.667 | 73.387 | −17.106 | 1.00 | 0.00 | C |
| ATOM | 3973 | OH | TYR | A 506 | 73.900 | 73.948 | −17.349 | 1.00 | 0.00 | O |
| ATOM | 3974 | N | ARG | A 507 | 69.435 | 68.498 | −16.378 | 1.00 | 0.00 | N |
| ATOM | 3975 | CA | ARG | A 507 | 70.383 | 67.397 | −16.214 | 1.00 | 0.00 | C |
| ATOM | 3976 | C | ARG | A 507 | 70.247 | 66.332 | −17.310 | 1.00 | 0.00 | C |
| ATOM | 3977 | O | ARG | A 507 | 71.241 | 65.774 | −17.775 | 1.00 | 0.00 | O |
| ATOM | 3978 | CB | ARG | A 507 | 70.196 | 66.750 | −14.837 | 1.00 | 0.00 | C |
| ATOM | 3979 | CG | ARG | A 507 | 71.242 | 65.699 | −14.490 | 1.00 | 0.00 | C |
| ATOM | 3980 | CD | ARG | A 507 | 71.042 | 65.179 | −13.074 | 1.00 | 0.00 | C |
| ATOM | 3981 | NE | ARG | A 507 | 72.059 | 64.198 | −12.700 | 1.00 | 0.00 | N |
| ATOM | 3982 | CZ | ARG | A 507 | 72.170 | 63.659 | −11.489 | 1.00 | 0.00 | C |
| ATOM | 3983 | NH1 | ARG | A 507 | 71.327 | 64.004 | −10.524 | 1.00 | 0.00 | N |
| ATOM | 3984 | NH2 | ARG | A 507 | 73.124 | 62.772 | −11.242 | 1.00 | 0.00 | N |
| ATOM | 3985 | N | LEU | A 508 | 69.016 | 66.061 | −17.725 | 1.00 | 0.00 | N |
| ATOM | 3986 | CA | LEU | A 508 | 68.760 | 65.056 | −18.752 | 1.00 | 0.00 | C |
| ATOM | 3987 | C | LEU | A 508 | 69.143 | 65.484 | −20.172 | 1.00 | 0.00 | C |
| ATOM | 3988 | O | LEU | A 508 | 69.398 | 64.634 | −21.029 | 1.00 | 0.00 | O |
| ATOM | 3989 | CB | LEU | A 508 | 67.277 | 64.664 | −18.739 | 1.00 | 0.00 | C |
| ATOM | 3990 | CG | LEU | A 508 | 66.779 | 63.779 | −17.590 | 1.00 | 0.00 | C |
| ATOM | 3991 | CD1 | LEU | A 508 | 65.251 | 63.830 | −17.527 | 1.00 | 0.00 | C |
| ATOM | 3992 | CD2 | LEU | A 508 | 67.265 | 62.350 | −17.791 | 1.00 | 0.00 | C |
| ATOM | 3993 | N | LEU | A 509 | 69.186 | 66.788 | −20.424 | 1.00 | 0.00 | N |
| ATOM | 3994 | CA | LEU | A 509 | 69.493 | 67.277 | −21.765 | 1.00 | 0.00 | C |
| ATOM | 3995 | C | LEU | A 509 | 70.764 | 68.106 | −21.916 | 1.00 | 0.00 | C |
| ATOM | 3996 | O | LEU | A 509 | 70.912 | 68.842 | −22.897 | 1.00 | 0.00 | O |
| ATOM | 3997 | CB | LEU | A 509 | 68.302 | 68.073 | −22.304 | 1.00 | 0.00 | C |
| ATOM | 3998 | CG | LEU | A 509 | 67.026 | 67.270 | −22.573 | 1.00 | 0.00 | C |
| ATOM | 3999 | CD1 | LEU | A 509 | 65.915 | 68.213 | −23.013 | 1.00 | 0.00 | C |
| ATOM | 4000 | CD2 | LEU | A 509 | 67.292 | 66.218 | −23.646 | 1.00 | 0.00 | C |
| ATOM | 4001 | N | THR | A 510 | 71.679 | 67.988 | −20.960 | 1.00 | 0.00 | N |
| ATOM | 4002 | CA | THR | A 510 | 72.936 | 68.728 | −21.017 | 1.00 | 0.00 | C |
| ATOM | 4003 | C | THR | A 510 | 74.098 | 67.740 | −21.024 | 1.00 | 0.00 | C |
| ATOM | 4004 | O | THR | A 510 | 74.104 | 66.782 | −20.251 | 1.00 | 0.00 | O |
| ATOM | 4005 | CB | THR | A 510 | 73.082 | 69.672 | −19.803 | 1.00 | 0.00 | C |
| ATOM | 4006 | OG1 | THR | A 510 | 71.994 | 70.604 | −19.794 | 1.00 | 0.00 | O |
| ATOM | 4007 | CG2 | THR | A 510 | 74.400 | 70.447 | −19.870 | 1.00 | 0.00 | C |
| ATOM | 4008 | N | LYS | A 511 | 75.073 | 67.961 | −21.902 | 1.00 | 0.00 | N |
| ATOM | 4009 | CA | LYS | A 511 | 76.231 | 67.068 | −21.971 | 1.00 | 0.00 | C |
| ATOM | 4010 | C | LYS | A 511 | 76.783 | 66.906 | −20.562 | 1.00 | 0.00 | C |
| ATOM | 4011 | O | LYS | A 511 | 77.065 | 67.890 | −19.875 | 1.00 | 0.00 | O |
| ATOM | 4012 | CB | LYS | A 511 | 77.309 | 67.641 | −22.900 | 1.00 | 0.00 | C |
| ATOM | 4013 | CG | LYS | A 511 | 78.522 | 66.738 | −23.040 | 1.00 | 0.00 | C |
| ATOM | 4014 | CD | LYS | A 511 | 79.568 | 67.314 | −23.977 | 1.00 | 0.00 | C |
| ATOM | 4015 | CE | LYS | A 511 | 80.772 | 66.384 | −24.062 | 1.00 | 0.00 | C |
| ATOM | 4016 | NZ | LYS | A 511 | 81.836 | 66.903 | −24.963 | 1.00 | 0.00 | N |
| ATOM | 4017 | N | PRO | A 512 | 76.940 | 65.653 | −20.107 | 1.00 | 0.00 | N |
| ATOM | 4018 | CA | PRO | A 512 | 77.450 | 65.348 | −18.768 | 1.00 | 0.00 | C |
| ATOM | 4019 | C | PRO | A 512 | 78.682 | 66.121 | −18.299 | 1.00 | 0.00 | C |
| ATOM | 4020 | O | PRO | A 512 | 78.699 | 66.639 | −17.184 | 1.00 | 0.00 | O |
| ATOM | 4021 | CB | PRO | A 512 | 77.698 | 63.843 | −18.834 | 1.00 | 0.00 | C |
| ATOM | 4022 | CG | PRO | A 512 | 76.615 | 63.379 | −19.750 | 1.00 | 0.00 | C |
| ATOM | 4023 | CD | PRO | A 512 | 76.668 | 64.412 | −20.855 | 1.00 | 0.00 | C |
| ATOM | 4024 | N | SER | A 513 | 79.709 | 66.198 | −19.139 | 1.00 | 0.00 | N |
| ATOM | 4025 | CA | SER | A 513 | 80.935 | 66.896 | −18.759 | 1.00 | 0.00 | C |
| ATOM | 4026 | C | SER | A 513 | 80.800 | 68.418 | −18.706 | 1.00 | 0.00 | C |
| ATOM | 4027 | O | SER | A 513 | 81.724 | 69.111 | −18.279 | 1.00 | 0.00 | O |
| ATOM | 4028 | CB | SER | A 513 | 82.081 | 66.499 | −19.701 | 1.00 | 0.00 | C |
| ATOM | 4029 | OG | SER | A 513 | 81.760 | 66.759 | −21.056 | 1.00 | 0.00 | O |
| ATOM | 4030 | N | ILE | A 514 | 79.647 | 68.931 | −19.131 | 1.00 | 0.00 | N |
| ATOM | 4031 | CA | ILE | A 514 | 79.380 | 70.369 | −19.127 | 1.00 | 0.00 | C |
| ATOM | 4032 | C | ILE | A 514 | 78.389 | 70.727 | −18.014 | 1.00 | 0.00 | C |
| ATOM | 4033 | O | ILE | A 514 | 78.412 | 71.836 | −17.481 | 1.00 | 0.00 | O |
| ATOM | 4034 | CB | ILE | A 514 | 78.790 | 70.831 | −20.492 | 1.00 | 0.00 | C |
| ATOM | 4035 | CG1 | ILE | A 514 | 79.849 | 70.697 | −21.589 | 1.00 | 0.00 | C |
| ATOM | 4036 | CG2 | ILE | A 514 | 78.302 | 72.276 | −20.401 | 1.00 | 0.00 | C |
| ATOM | 4037 | CD1 | ILE | A 514 | 79.354 | 71.076 | −22.974 | 1.00 | 0.00 | C |
| ATOM | 4038 | N | TYR | A 515 | 77.525 | 69.775 | −17.671 | 1.00 | 0.00 | N |
| ATOM | 4039 | CA | TYR | A 515 | 76.511 | 69.962 | −16.630 | 1.00 | 0.00 | C |
| ATOM | 4040 | C | TYR | A 515 | 77.099 | 70.524 | −15.330 | 1.00 | 0.00 | C |
| ATOM | 4041 | O | TYR | A 515 | 77.925 | 69.883 | −14.681 | 1.00 | 0.00 | O |
| ATOM | 4042 | CB | TYR | A 515 | 75.817 | 68.621 | −16.373 | 1.00 | 0.00 | C |
| ATOM | 4043 | CG | TYR | A 515 | 74.779 | 68.618 | −15.276 | 1.00 | 0.00 | C |
| ATOM | 4044 | CD1 | TYR | A 515 | 73.625 | 69.397 | −15.369 | 1.00 | 0.00 | C |
| ATOM | 4045 | CD2 | TYR | A 515 | 74.936 | 67.802 | −14.157 | 1.00 | 0.00 | C |
| ATOM | 4046 | CE1 | TYR | A 515 | 72.648 | 69.359 | −14.368 | 1.00 | 0.00 | C |
| ATOM | 4047 | CE2 | TYR | A 515 | 73.974 | 67.754 | −13.157 | 1.00 | 0.00 | C |
| ATOM | 4048 | CZ | TYR | A 515 | 72.832 | 68.533 | −13.265 | 1.00 | 0.00 | C |
| ATOM | 4049 | OH | TYR | A 515 | 71.881 | 68.475 | −12.270 | 1.00 | 0.00 | O |
| ATOM | 4050 | N | SER | A 516 | 76.665 | 71.728 | −14.959 | 1.00 | 0.00 | N |
| ATOM | 4051 | CA | SER | A 516 | 77.141 | 72.404 | −13.746 | 1.00 | 0.00 | C |
| ATOM | 4052 | C | SER | A 516 | 75.931 | 73.040 | −13.062 | 1.00 | 0.00 | C |
| ATOM | 4053 | O | SER | A 516 | 75.685 | 74.243 | −13.188 | 1.00 | 0.00 | O |
| ATOM | 4054 | CB | SER | A 516 | 78.159 | 73.485 | −14.121 | 1.00 | 0.00 | C |
| ATOM | 4055 | OG | SER | A 516 | 78.766 | 74.044 | −12.969 | 1.00 | 0.00 | O |
| ATOM | 4056 | N | PRO | A 517 | 75.166 | 72.236 | −12.310 | 1.00 | 0.00 | N |
| ATOM | 4057 | CA | PRO | A 517 | 73.967 | 72.700 | −11.614 | 1.00 | 0.00 | C |
| ATOM | 4058 | C | PRO | A 517 | 74.064 | 73.562 | −10.365 | 1.00 | 0.00 | C |
| ATOM | 4059 | O | PRO | A 517 | 74.917 | 73.360 | −9.503 | 1.00 | 0.00 | O |
| ATOM | 4060 | CB | PRO | A 517 | 73.229 | 71.399 | −11.323 | 1.00 | 0.00 | C |
| ATOM | 4061 | CG | PRO | A 517 | 74.356 | 70.483 | −10.991 | 1.00 | 0.00 | C |
| ATOM | 4062 | CD | PRO | A 517 | 75.355 | 70.788 | −12.102 | 1.00 | 0.00 | C |
| ATOM | 4063 | N | ASP | A 518 | 73.153 | 74.528 | −10.303 | 1.00 | 0.00 | N |
| ATOM | 4064 | CA | ASP | A 518 | 72.978 | 75.418 | −9.164 | 1.00 | 0.00 | C |
| ATOM | 4065 | C | ASP | A 518 | 71.560 | 74.999 | −8.789 | 1.00 | 0.00 | C |
| ATOM | 4066 | O | ASP | A 518 | 70.597 | 75.413 | −9.434 | 1.00 | 0.00 | O |
| ATOM | 4067 | CB | ASP | A 518 | 72.969 | 76.885 | −9.586 | 1.00 | 0.00 | C |
| ATOM | 4068 | CG | ASP | A 518 | 72.657 | 77.819 | −8.426 | 1.00 | 0.00 | C |
| ATOM | 4069 | OD1 | ASP | A 518 | 72.003 | 77.372 | −7.457 | 1.00 | 0.00 | O |
| ATOM | 4070 | OD2 | ASP | A 518 | 73.051 | 79.001 | −8.487 | 1.00 | 0.00 | O |
| ATOM | 4071 | N | PHE | A 519 | 71.437 | 74.166 | −7.764 | 1.00 | 0.00 | N |
| ATOM | 4072 | CA | PHE | A 519 | 70.141 | 73.644 | −7.352 | 1.00 | 0.00 | C |
| ATOM | 4073 | C | PHE | A 519 | 69.077 | 74.654 | −6.947 | 1.00 | 0.00 | C |
| ATOM | 4074 | O | PHE | A 519 | 67.931 | 74.279 | −6.698 | 1.00 | 0.00 | O |
| ATOM | 4075 | CB | PHE | A 519 | 70.341 | 72.608 | −6.246 | 1.00 | 0.00 | C |
| ATOM | 4076 | CG | PHE | A 519 | 71.207 | 71.448 | −6.664 | 1.00 | 0.00 | C |
| ATOM | 4077 | CD1 | PHE | A 519 | 70.948 | 70.765 | −7.851 | 1.00 | 0.00 | C |
| ATOM | 4078 | CD2 | PHE | A 519 | 72.287 | 71.049 | −5.884 | 1.00 | 0.00 | C |
| ATOM | 4079 | CE1 | PHE | A 519 | 71.754 | 69.700 | −8.257 | 1.00 | 0.00 | C |
| ATOM | 4080 | CE2 | PHE | A 519 | 73.098 | 69.987 | −6.280 | 1.00 | 0.00 | C |
| ATOM | 4081 | CZ | PHE | A 519 | 72.832 | 69.311 | −7.469 | 1.00 | 0.00 | C |
| ATOM | 4082 | N | SER | A 520 | 69.439 | 75.931 | −6.902 | 1.00 | 0.00 | N |
| ATOM | 4083 | CA | SER | A 520 | 68.483 | 76.973 | −6.535 | 1.00 | 0.00 | C |
| ATOM | 4084 | C | SER | A 520 | 68.133 | 77.823 | −7.754 | 1.00 | 0.00 | C |
| ATOM | 4085 | O | SER | A 520 | 67.260 | 78.688 | −7.691 | 1.00 | 0.00 | O |
| ATOM | 4086 | CB | SER | A 520 | 69.073 | 77.879 | −5.453 | 1.00 | 0.00 | C |
| ATOM | 4087 | OG | SER | A 520 | 70.132 | 78.665 | −5.978 | 1.00 | 0.00 | O |
| ATOM | 4088 | N | PHE | A 521 | 68.822 | 77.565 | −8.860 | 1.00 | 0.00 | N |
| ATOM | 4089 | CA | PHE | A 521 | 68.629 | 78.317 | −10.095 | 1.00 | 0.00 | C |
| ATOM | 4090 | C | PHE | A 521 | 67.500 | 77.774 | −10.970 | 1.00 | 0.00 | C |
| ATOM | 4091 | O | PHE | A 521 | 67.237 | 76.574 | −10.990 | 1.00 | 0.00 | O |
| ATOM | 4092 | CB | PHE | A 521 | 69.936 | 78.319 | −10.895 | 1.00 | 0.00 | C |
| ATOM | 4093 | CG | PHE | A 521 | 69.952 | 79.299 | −12.034 | 1.00 | 0.00 | C |
| ATOM | 4094 | CD1 | PHE | A 521 | 70.084 | 80.664 | −11.790 | 1.00 | 0.00 | C |
| ATOM | 4095 | CD2 | PHE | A 521 | 69.812 | 78.862 | −13.348 | 1.00 | 0.00 | C |
| ATOM | 4096 | CE1 | PHE | A 521 | 70.075 | 81.583 | −12.842 | 1.00 | 0.00 | C |
| ATOM | 4097 | CE2 | PHE | A 521 | 69.801 | 79.768 | −14.406 | 1.00 | 0.00 | C |
| ATOM | 4098 | CZ | PHE | A 521 | 69.932 | 81.133 | −14.152 | 1.00 | 0.00 | C |
| ATOM | 4099 | N | SER | A 522 | 66.837 | 78.671 | −11.697 | 1.00 | 0.00 | N |
| ATOM | 4100 | CA | SER | A 522 | 65.753 | 78.280 | −12.590 | 1.00 | 0.00 | C |
| ATOM | 4101 | C | SER | A 522 | 66.264 | 78.252 | −14.027 | 1.00 | 0.00 | C |
| ATOM | 4102 | O | SER | A 522 | 66.320 | 79.286 | −14.692 | 1.00 | 0.00 | O |
| ATOM | 4103 | CB | SER | A 522 | 64.580 | 79.261 | −12.477 | 1.00 | 0.00 | C |
| ATOM | 4104 | OG | SER | A 522 | 63.927 | 79.133 | −11.224 | 1.00 | 0.00 | O |
| ATOM | 4105 | N | TYR | A 523 | 66.645 | 77.070 | −14.497 | 1.00 | 0.00 | N |
| ATOM | 4106 | CA | TYR | A 523 | 67.149 | 76.919 | −15.857 | 1.00 | 0.00 | C |
| ATOM | 4107 | C | TYR | A 523 | 66.025 | 77.000 | −16.868 | 1.00 | 0.00 | C |
| ATOM | 4108 | O | TYR | A 523 | 66.228 | 77.440 | −18.002 | 1.00 | 0.00 | O |
| ATOM | 4109 | CB | TYR | A 523 | 67.885 | 75.586 | −16.013 | 1.00 | 0.00 | C |
| ATOM | 4110 | CG | TYR | A 523 | 69.160 | 75.523 | −15.211 | 1.00 | 0.00 | C |
| ATOM | 4111 | CD1 | TYR | A 523 | 69.188 | 74.932 | −13.949 | 1.00 | 0.00 | C |
| ATOM | 4112 | CD2 | TYR | A 523 | 70.335 | 76.106 | −15.695 | 1.00 | 0.00 | C |
| ATOM | 4113 | CE1 | TYR | A 523 | 70.358 | 74.926 | −13.187 | 1.00 | 0.00 | C |
| ATOM | 4114 | CE2 | TYR | A 523 | 71.498 | 76.108 | −14.945 | 1.00 | 0.00 | C |
| ATOM | 4115 | CZ | TYR | A 523 | 71.506 | 75.519 | −13.693 | 1.00 | 0.00 | C |
| ATOM | 4116 | OH | TYR | A 523 | 72.660 | 75.542 | −12.944 | 1.00 | 0.00 | O |
| ATOM | 4117 | N | PHE | A 524 | 64.840 | 76.559 | −16.456 | 1.00 | 0.00 | N |
| ATOM | 4118 | CA | PHE | A 524 | 63.665 | 76.603 | −17.316 | 1.00 | 0.00 | C |
| ATOM | 4119 | C | PHE | A 524 | 62.464 | 77.104 | −16.539 | 1.00 | 0.00 | C |
| ATOM | 4120 | O | PHE | A 524 | 62.401 | 76.986 | −15.315 | 1.00 | 0.00 | O |
| ATOM | 4121 | CB | PHE | A 524 | 63.293 | 75.221 | −17.857 | 1.00 | 0.00 | C |
| ATOM | 4122 | CG | PHE | A 524 | 64.335 | 74.593 | −18.726 | 1.00 | 0.00 | C |
| ATOM | 4123 | CD1 | PHE | A 524 | 65.402 | 73.902 | −18.165 | 1.00 | 0.00 | C |
| ATOM | 4124 | CD2 | PHE | A 524 | 64.226 | 74.654 | −20.114 | 1.00 | 0.00 | C |
| ATOM | 4125 | CE1 | PHE | A 524 | 66.348 | 73.276 | −18.972 | 1.00 | 0.00 | C |
| ATOM | 4126 | CE2 | PHE | A 524 | 65.166 | 74.031 | −20.931 | 1.00 | 0.00 | C |
| ATOM | 4127 | CZ | PHE | A 524 | 66.230 | 73.338 | −20.355 | 1.00 | 0.00 | C |
| ATOM | 4128 | N | THR | A 525 | 61.509 | 77.656 | −17.275 | 1.00 | 0.00 | N |
| ATOM | 4129 | CA | THR | A 525 | 60.265 | 78.139 | −16.707 | 1.00 | 0.00 | C |
| ATOM | 4130 | C | THR | A 525 | 59.180 | 77.306 | −17.378 | 1.00 | 0.00 | C |
| ATOM | 4131 | O | THR | A 525 | 59.191 | 77.145 | −18.598 | 1.00 | 0.00 | O |
| ATOM | 4132 | CB | THR | A 525 | 60.028 | 79.623 | −17.042 | 1.00 | 0.00 | C |
| ATOM | 4133 | OG1 | THR | A 525 | 61.030 | 80.423 | −16.402 | 1.00 | 0.00 | O |
| ATOM | 4134 | CG2 | THR | A 525 | 58.659 | 80.065 | −16.561 | 1.00 | 0.00 | C |
| ATOM | 4135 | N | LEU | A 526 | 58.262 | 76.758 | −16.590 | 1.00 | 0.00 | N |
| ATOM | 4136 | CA | LEU | A 526 | 57.180 | 75.972 | −17.164 | 1.00 | 0.00 | C |
| ATOM | 4137 | C | LEU | A 526 | 56.183 | 76.911 | −17.838 | 1.00 | 0.00 | C |
| ATOM | 4138 | O | LEU | A 526 | 55.925 | 78.011 | −17.348 | 1.00 | 0.00 | O |
| ATOM | 4139 | CB | LEU | A 526 | 56.452 | 75.170 | −16.079 | 1.00 | 0.00 | C |
| ATOM | 4140 | CG | LEU | A 526 | 57.008 | 73.807 | −15.666 | 1.00 | 0.00 | C |
| ATOM | 4141 | CD1 | LEU | A 526 | 56.224 | 73.280 | −14.470 | 1.00 | 0.00 | C |
| ATOM | 4142 | CD2 | LEU | A 526 | 56.907 | 72.838 | −16.833 | 1.00 | 0.00 | C |
| ATOM | 4143 | N | ASP | A 527 | 55.642 | 76.483 | −18.973 | 1.00 | 0.00 | N |
| ATOM | 4144 | CA | ASP | A 527 | 54.646 | 77.274 | −19.676 | 1.00 | 0.00 | C |
| ATOM | 4145 | C | ASP | A 527 | 53.404 | 76.400 | −19.756 | 1.00 | 0.00 | C |
| ATOM | 4146 | O | ASP | A 527 | 53.425 | 75.350 | −20.389 | 1.00 | 0.00 | O |
| ATOM | 4147 | CB | ASP | A 527 | 55.121 | 77.635 | −21.086 | 1.00 | 0.00 | C |
| ATOM | 4148 | CG | ASP | A 527 | 54.134 | 78.528 | −21.818 | 1.00 | 0.00 | C |
| ATOM | 4149 | OD1 | ASP | A 527 | 53.867 | 79.646 | −21.324 | 1.00 | 0.00 | O |
| ATOM | 4150 | OD2 | ASP | A 527 | 53.622 | 78.110 | −22.881 | 1.00 | 0.00 | O |
| ATOM | 4151 | N | ASP | A 528 | 52.337 | 76.824 | −19.089 | 1.00 | 0.00 | N |
| ATOM | 4152 | CA | ASP | A 528 | 51.084 | 76.075 | −19.076 | 1.00 | 0.00 | C |
| ATOM | 4153 | C | ASP | A 528 | 50.010 | 76.962 | −19.688 | 1.00 | 0.00 | C |
| ATOM | 4154 | O | ASP | A 528 | 49.667 | 78.007 | −19.139 | 1.00 | 0.00 | O |
| ATOM | 4155 | CB | ASP | A 528 | 50.704 | 75.713 | −17.637 | 1.00 | 0.00 | C |
| ATOM | 4156 | CG | ASP | A 528 | 49.666 | 74.605 | −17.564 | 1.00 | 0.00 | C |
| ATOM | 4157 | OD1 | ASP | A 528 | 48.653 | 74.670 | −18.294 | 1.00 | 0.00 | O |
| ATOM | 4158 | OD2 | ASP | A 528 | 49.865 | 73.667 | −16.766 | 1.00 | 0.00 | O |
| ATOM | 4159 | N | SER | A 529 | 49.474 | 76.548 | −20.827 | 1.00 | 0.00 | N |
| ATOM | 4160 | CA | SER | A 529 | 48.465 | 77.358 | −21.491 | 1.00 | 0.00 | C |
| ATOM | 4161 | C | SER | A 529 | 47.059 | 77.161 | −20.935 | 1.00 | 0.00 | C |
| ATOM | 4162 | O | SER | A 529 | 46.154 | 77.916 | −21.277 | 1.00 | 0.00 | O |
| ATOM | 4163 | CB | SER | A 529 | 48.469 | 77.071 | −22.995 | 1.00 | 0.00 | C |
| ATOM | 4164 | OG | SER | A 529 | 47.890 | 75.810 | −23.261 | 1.00 | 0.00 | O |
| ATOM | 4165 | N | ARG | A 530 | 46.869 | 76.167 | −20.071 | 1.00 | 0.00 | N |
| ATOM | 4166 | CA | ARG | A 530 | 45.536 | 75.925 | −19.536 | 1.00 | 0.00 | C |
| ATOM | 4167 | C | ARG | A 530 | 45.351 | 76.074 | −18.030 | 1.00 | 0.00 | C |
| ATOM | 4168 | O | ARG | A 530 | 44.280 | 75.778 | −17.502 | 1.00 | 0.00 | O |
| ATOM | 4169 | CB | ARG | A 530 | 45.036 | 74.560 | −20.015 | 1.00 | 0.00 | C |
| ATOM | 4170 | CG | ARG | A 530 | 44.929 | 74.517 | −21.531 | 1.00 | 0.00 | C |
| ATOM | 4171 | CD | ARG | A 530 | 44.292 | 73.244 | −22.057 | 1.00 | 0.00 | C |
| ATOM | 4172 | NE | ARG | A 530 | 45.038 | 72.051 | −21.670 | 1.00 | 0.00 | N |
| ATOM | 4173 | CZ | ARG | A 530 | 44.816 | 70.843 | −22.178 | 1.00 | 0.00 | C |
| ATOM | 4174 | NH1 | ARG | A 530 | 43.869 | 70.678 | −23.093 | 1.00 | 0.00 | N |
| ATOM | 4175 | NH2 | ARG | A 530 | 45.535 | 69.801 | −21.771 | 1.00 | 0.00 | N |
| ATOM | 4176 | N | TRP | A 531 | 46.389 | 76.531 | −17.337 | 1.00 | 0.00 | N |
| ATOM | 4177 | CA | TRP | A 531 | 46.277 | 76.764 | −15.901 | 1.00 | 0.00 | C |
| ATOM | 4178 | C | TRP | A 531 | 47.246 | 77.837 | −15.433 | 1.00 | 0.00 | C |
| ATOM | 4179 | O | TRP | A 531 | 48.444 | 77.753 | −15.695 | 1.00 | 0.00 | O |
| ATOM | 4180 | CB | TRP | A 531 | 46.519 | 75.493 | −15.085 | 1.00 | 0.00 | C |
| ATOM | 4181 | CG | TRP | A 531 | 46.418 | 75.781 | −13.618 | 1.00 | 0.00 | C |
| ATOM | 4182 | CD1 | TRP | A 531 | 47.438 | 76.128 | −12.773 | 1.00 | 0.00 | C |
| ATOM | 4183 | CD2 | TRP | A 531 | 45.215 | 75.860 | −12.846 | 1.00 | 0.00 | C |
| ATOM | 4184 | NE1 | TRP | A 531 | 46.941 | 76.422 | −11.524 | 1.00 | 0.00 | N |
| ATOM | 4185 | CE2 | TRP | A 531 | 45.580 | 76.265 | −11.542 | 1.00 | 0.00 | C |
| ATOM | 4186 | CE3 | TRP | A 531 | 43.864 | 75.631 | −13.130 | 1.00 | 0.00 | C |
| ATOM | 4187 | CZ2 | TRP | A 531 | 44.638 | 76.446 | −10.522 | 1.00 | 0.00 | C |
| ATOM | 4188 | CZ3 | TRP | A 531 | 42.925 | 75.813 | −12.114 | 1.00 | 0.00 | C |
| ATOM | 4189 | CH2 | TRP | A 531 | 43.319 | 76.216 | −10.828 | 1.00 | 0.00 | C |
| ATOM | 4190 | N | PRO | A 532 | 46.738 | 78.857 | −14.722 | 1.00 | 0.00 | N |
| ATOM | 4191 | CA | PRO | A 532 | 45.325 | 79.023 | −14.358 | 1.00 | 0.00 | C |
| ATOM | 4192 | C | PRO | A 532 | 44.419 | 79.228 | −15.567 | 1.00 | 0.00 | C |
| ATOM | 4193 | O | PRO | A 532 | 43.200 | 79.075 | −15.473 | 1.00 | 0.00 | O |
| ATOM | 4194 | CB | PRO | A 532 | 45.348 | 80.240 | −13.433 | 1.00 | 0.00 | C |
| ATOM | 4195 | CG | PRO | A 532 | 46.700 | 80.135 | −12.787 | 1.00 | 0.00 | C |
| ATOM | 4196 | CD | PRO | A 532 | 47.576 | 79.816 | −13.980 | 1.00 | 0.00 | C |
| ATOM | 4197 | N | GLY | A 533 | 45.022 | 79.572 | −16.700 | 1.00 | 0.00 | N |
| ATOM | 4198 | CA | GLY | A 533 | 44.255 | 79.771 | −17.914 | 1.00 | 0.00 | C |
| ATOM | 4199 | C | GLY | A 533 | 44.067 | 81.220 | −18.313 | 1.00 | 0.00 | C |
| ATOM | 4200 | O | GLY | A 533 | 44.049 | 82.116 | −17.466 | 1.00 | 0.00 | O |
| ATOM | 4201 | N | SER | A 534 | 43.933 | 81.449 | −19.616 | 1.00 | 0.00 | N |
| ATOM | 4202 | CA | SER | A 534 | 43.734 | 82.792 | −20.147 | 1.00 | 0.00 | C |
| ATOM | 4203 | C | SER | A 534 | 42.442 | 83.369 | −19.578 | 1.00 | 0.00 | C |
| ATOM | 4204 | O | SER | A 534 | 41.400 | 82.711 | −19.598 | 1.00 | 0.00 | O |
| ATOM | 4205 | CB | SER | A 534 | 43.655 | 82.744 | −21.675 | 1.00 | 0.00 | C |
| ATOM | 4206 | OG | SER | A 534 | 43.324 | 84.013 | −22.208 | 1.00 | 0.00 | O |
| ATOM | 4207 | N | GLY | A 535 | 42.514 | 84.595 | −19.069 | 1.00 | 0.00 | N |
| ATOM | 4208 | CA | GLY | A 535 | 41.337 | 85.223 | −18.498 | 1.00 | 0.00 | C |
| ATOM | 4209 | C | GLY | A 535 | 41.207 | 84.928 | −17.014 | 1.00 | 0.00 | C |
| ATOM | 4210 | O | GLY | A 535 | 40.351 | 85.491 | −16.330 | 1.00 | 0.00 | O |
| ATOM | 4211 | N | VAL | A 536 | 42.056 | 84.035 | −16.516 | 1.00 | 0.00 | N |
| ATOM | 4212 | CA | VAL | A 536 | 42.040 | 83.669 | −15.105 | 1.00 | 0.00 | C |
| ATOM | 4213 | C | VAL | A 536 | 43.218 | 84.336 | −14.400 | 1.00 | 0.00 | C |
| ATOM | 4214 | O | VAL | A 536 | 43.078 | 84.885 | −13.307 | 1.00 | 0.00 | O |
| ATOM | 4215 | CB | VAL | A 536 | 42.147 | 82.134 | −14.925 | 1.00 | 0.00 | C |
| ATOM | 4216 | CG1 | VAL | A 536 | 42.091 | 81.774 | −13.448 | 1.00 | 0.00 | C |
| ATOM | 4217 | CG2 | VAL | A 536 | 41.022 | 81.443 | −15.682 | 1.00 | 0.00 | C |
| ATOM | 4218 | N | GLU | A 537 | 44.378 | 84.292 | −15.046 | 1.00 | 0.00 | N |
| ATOM | 4219 | CA | GLU | A 537 | 45.590 | 84.883 | −14.495 | 1.00 | 0.00 | C |
| ATOM | 4220 | C | GLU | A 537 | 46.675 | 84.972 | −15.565 | 1.00 | 0.00 | C |
| ATOM | 4221 | O | GLU | A 537 | 47.086 | 83.956 | −16.126 | 1.00 | 0.00 | O |
| ATOM | 4222 | CB | GLU | A 537 | 46.083 | 84.040 | −13.310 | 1.00 | 0.00 | C |
| ATOM | 4223 | CG | GLU | A 537 | 47.529 | 84.290 | −12.893 | 1.00 | 0.00 | C |
| ATOM | 4224 | CD | GLU | A 537 | 47.936 | 83.485 | −11.665 | 1.00 | 0.00 | C |
| ATOM | 4225 | OE1 | GLU | A 537 | 49.153 | 83.284 | −11.457 | 1.00 | 0.00 | O |
| ATOM | 4226 | OE2 | GLU | A 537 | 47.042 | 83.058 | −10.902 | 1.00 | 0.00 | O |
| ATOM | 4227 | N | ASP | A 538 | 47.126 | 86.188 | −15.863 | 1.00 | 0.00 | N |
| ATOM | 4228 | CA | ASP | A 538 | 48.182 | 86.365 | −16.855 | 1.00 | 0.00 | C |
| ATOM | 4229 | C | ASP | A 538 | 49.474 | 85.975 | −16.153 | 1.00 | 0.00 | C |
| ATOM | 4230 | O | ASP | A 538 | 50.127 | 86.806 | −15.521 | 1.00 | 0.00 | O |
| ATOM | 4231 | CB | ASP | A 538 | 48.247 | 87.822 | −17.326 | 1.00 | 0.00 | C |
| ATOM | 4232 | CG | ASP | A 538 | 49.236 | 88.022 | −18.463 | 1.00 | 0.00 | C |
| ATOM | 4233 | OD1 | ASP | A 538 | 49.147 | 87.282 | −19.466 | 1.00 | 0.00 | O |
| ATOM | 4234 | OD2 | ASP | A 538 | 50.100 | 88.919 | −18.357 | 1.00 | 0.00 | O |
| ATOM | 4235 | N | SER | A 539 | 49.832 | 84.700 | −16.261 | 1.00 | 0.00 | N |
| ATOM | 4236 | CA | SER | A 539 | 51.021 | 84.179 | −15.598 | 1.00 | 0.00 | C |
| ATOM | 4237 | C | SER | A 539 | 52.142 | 83.720 | −16.521 | 1.00 | 0.00 | C |
| ATOM | 4238 | O | SER | A 539 | 53.318 | 83.855 | −16.183 | 1.00 | 0.00 | O |
| ATOM | 4239 | CB | SER | A 539 | 50.626 | 83.013 | −14.690 | 1.00 | 0.00 | C |
| ATOM | 4240 | OG | SER | A 539 | 50.051 | 81.962 | −15.450 | 1.00 | 0.00 | O |
| ATOM | 4241 | N | ARG | A 540 | 51.790 | 83.166 | −17.676 | 1.00 | 0.00 | N |
| ATOM | 4242 | CA | ARG | A 540 | 52.814 | 82.682 | −18.591 | 1.00 | 0.00 | C |
| ATOM | 4243 | C | ARG | A 540 | 53.684 | 83.795 | −19.160 | 1.00 | 0.00 | C |
| ATOM | 4244 | O | ARG | A 540 | 53.211 | 84.890 | −19.469 | 1.00 | 0.00 | O |
| ATOM | 4245 | CB | ARG | A 540 | 52.184 | 81.839 | −19.706 | 1.00 | 0.00 | C |
| ATOM | 4246 | CG | ARG | A 540 | 50.945 | 82.418 | −20.342 | 1.00 | 0.00 | C |
| ATOM | 4247 | CD | ARG | A 540 | 50.190 | 81.336 | −21.119 | 1.00 | 0.00 | C |
| ATOM | 4248 | NE | ARG | A 540 | 51.054 | 80.613 | −22.053 | 1.00 | 0.00 | N |
| ATOM | 4249 | CZ | ARG | A 540 | 50.624 | 80.055 | −23.181 | 1.00 | 0.00 | C |
| ATOM | 4250 | NH1 | ARG | A 540 | 49.342 | 80.137 | −23.515 | 1.00 | 0.00 | N |
| ATOM | 4251 | NH2 | ARG | A 540 | 51.474 | 79.423 | −23.978 | 1.00 | 0.00 | N |
| ATOM | 4252 | N | THR | A 541 | 54.971 | 83.497 | −19.280 | 1.00 | 0.00 | N |
| ATOM | 4253 | CA | THR | A 541 | 55.945 | 84.453 | −19.773 | 1.00 | 0.00 | C |
| ATOM | 4254 | C | THR | A 541 | 55.965 | 84.548 | −21.289 | 1.00 | 0.00 | C |
| ATOM | 4255 | O | THR | A 541 | 55.590 | 83.610 | −21.992 | 1.00 | 0.00 | O |
| ATOM | 4256 | CB | THR | A 541 | 57.366 | 84.077 | −19.317 | 1.00 | 0.00 | C |
| ATOM | 4257 | OG1 | THR | A 541 | 57.867 | 83.026 | −20.151 | 1.00 | 0.00 | O |
| ATOM | 4258 | CG2 | THR | A 541 | 57.356 | 83.592 | −17.874 | 1.00 | 0.00 | C |
| ATOM | 4259 | N | THR | A 542 | 56.405 | 85.699 | −21.783 | 1.00 | 0.00 | N |
| ATOM | 4260 | CA | THR | A 542 | 56.517 | 85.919 | −23.213 | 1.00 | 0.00 | C |
| ATOM | 4261 | C | THR | A 542 | 57.974 | 85.685 | −23.579 | 1.00 | 0.00 | C |
| ATOM | 4262 | O | THR | A 542 | 58.879 | 86.159 | −22.889 | 1.00 | 0.00 | O |
| ATOM | 4263 | CB | THR | A 542 | 56.159 | 87.373 | −23.610 | 1.00 | 0.00 | C |
| ATOM | 4264 | OG1 | THR | A 542 | 54.781 | 87.629 | −23.322 | 1.00 | 0.00 | O |
| ATOM | 4265 | CG2 | THR | A 542 | 56.411 | 87.593 | −25.099 | 1.00 | 0.00 | C |
| ATOM | 4266 | N | ILE | A 543 | 58.204 | 84.924 | −24.640 | 1.00 | 0.00 | N |
| ATOM | 4267 | CA | ILE | A 543 | 59.564 | 84.693 | −25.092 | 1.00 | 0.00 | C |
| ATOM | 4268 | C | ILE | A 543 | 59.896 | 85.966 | −25.866 | 1.00 | 0.00 | C |
| ATOM | 4269 | O | ILE | A 543 | 59.314 | 86.226 | −26.921 | 1.00 | 0.00 | O |
| ATOM | 4270 | CB | ILE | A 543 | 59.647 | 83.466 | −26.017 | 1.00 | 0.00 | C |
| ATOM | 4271 | CG1 | ILE | A 543 | 59.334 | 82.200 | −25.208 | 1.00 | 0.00 | C |
| ATOM | 4272 | CG2 | ILE | A 543 | 61.031 | 83.380 | −26.659 | 1.00 | 0.00 | C |
| ATOM | 4273 | CD1 | ILE | A 543 | 59.377 | 80.920 | −26.014 | 1.00 | 0.00 | C |
| ATOM | 4274 | N | ILE | A 544 | 60.803 | 86.769 | −25.318 | 1.00 | 0.00 | N |
| ATOM | 4275 | CA | ILE | A 544 | 61.184 | 88.030 | −25.937 | 1.00 | 0.00 | C |
| ATOM | 4276 | C | ILE | A 544 | 62.405 | 87.881 | −26.835 | 1.00 | 0.00 | C |
| ATOM | 4277 | O | ILE | A 544 | 63.512 | 87.604 | −26.371 | 1.00 | 0.00 | O |
| ATOM | 4278 | CB | ILE | A 544 | 61.448 | 89.105 | −24.857 | 1.00 | 0.00 | C |
| ATOM | 4279 | CG1 | ILE | A 544 | 60.187 | 89.289 | −24.007 | 1.00 | 0.00 | C |
| ATOM | 4280 | CG2 | ILE | A 544 | 61.833 | 90.427 | −25.508 | 1.00 | 0.00 | C |
| ATOM | 4281 | CD1 | ILE | A 544 | 60.306 | 90.354 | −22.939 | 1.00 | 0.00 | C |
| ATOM | 4282 | N | LEU | A 545 | 62.183 | 88.057 | −28.132 | 1.00 | 0.00 | N |
| ATOM | 4283 | CA | LEU | A 545 | 63.245 | 87.944 | −29.121 | 1.00 | 0.00 | C |
| ATOM | 4284 | C | LEU | A 545 | 63.407 | 89.277 | −29.850 | 1.00 | 0.00 | C |
| ATOM | 4285 | O | LEU | A 545 | 62.465 | 90.060 | −29.937 | 1.00 | 0.00 | O |
| ATOM | 4286 | CB | LEU | A 545 | 62.902 | 86.840 | −30.124 | 1.00 | 0.00 | C |
| ATOM | 4287 | CG | LEU | A 545 | 62.592 | 85.457 | −29.537 | 1.00 | 0.00 | C |
| ATOM | 4288 | CD1 | LEU | A 545 | 62.141 | 84.522 | −30.649 | 1.00 | 0.00 | C |
| ATOM | 4289 | CD2 | LEU | A 545 | 63.828 | 84.901 | −28.829 | 1.00 | 0.00 | C |
| ATOM | 4290 | N | GLY | A 546 | 64.604 | 89.530 | −30.368 | 1.00 | 0.00 | N |
| ATOM | 4291 | CA | GLY | A 546 | 64.852 | 90.770 | −31.083 | 1.00 | 0.00 | C |
| ATOM | 4292 | C | GLY | A 546 | 66.276 | 90.850 | −31.597 | 1.00 | 0.00 | C |
| ATOM | 4293 | O | GLY | A 546 | 67.201 | 90.370 | −30.949 | 1.00 | 0.00 | O |
| ATOM | 4294 | N | GLU | A 547 | 66.454 | 91.465 | −32.761 | 1.00 | 0.00 | N |
| ATOM | 4295 | CA | GLU | A 547 | 67.777 | 91.600 | −33.364 | 1.00 | 0.00 | C |
| ATOM | 4296 | C | GLU | A 547 | 68.770 | 92.241 | −32.401 | 1.00 | 0.00 | C |
| ATOM | 4297 | O | GLU | A 547 | 69.947 | 91.881 | −32.373 | 1.00 | 0.00 | O |
| ATOM | 4298 | CB | GLU | A 547 | 67.696 | 92.459 | −34.628 | 1.00 | 0.00 | C |
| ATOM | 4299 | CG | GLU | A 547 | 66.676 | 91.990 | −35.651 | 1.00 | 0.00 | C |
| ATOM | 4300 | CD | GLU | A 547 | 66.538 | 92.962 | −36.812 | 1.00 | 0.00 | C |
| ATOM | 4301 | OE1 | GLU | A 547 | 67.527 | 93.141 | −37.558 | 1.00 | 0.00 | O |
| ATOM | 4302 | OE2 | GLU | A 547 | 65.444 | 93.551 | −36.972 | 1.00 | 0.00 | O |
| ATOM | 4303 | N | ASP | A 548 | 68.287 | 93.193 | −31.611 | 1.00 | 0.00 | N |
| ATOM | 4304 | CA | ASP | A 548 | 69.140 | 93.901 | −30.667 | 1.00 | 0.00 | C |
| ATOM | 4305 | C | ASP | A 548 | 69.165 | 93.319 | −29.256 | 1.00 | 0.00 | C |
| ATOM | 4306 | O | ASP | A 548 | 69.748 | 93.923 | −28.353 | 1.00 | 0.00 | O |
| ATOM | 4307 | CB | ASP | A 548 | 68.720 | 95.372 | −30.590 | 1.00 | 0.00 | C |
| ATOM | 4308 | CG | ASP | A 548 | 68.921 | 96.106 | −31.900 | 1.00 | 0.00 | C |
| ATOM | 4309 | OD1 | ASP | A 548 | 70.067 | 96.147 | −32.390 | 1.00 | 0.00 | O |
| ATOM | 4310 | OD2 | ASP | A 548 | 67.933 | 96.644 | −32.439 | 1.00 | 0.00 | O |
| ATOM | 4311 | N | ILE | A 549 | 68.556 | 92.153 | −29.055 | 1.00 | 0.00 | N |
| ATOM | 4312 | CA | ILE | A 549 | 68.541 | 91.570 | −27.716 | 1.00 | 0.00 | C |
| ATOM | 4313 | C | ILE | A 549 | 68.763 | 90.060 | −27.658 | 1.00 | 0.00 | C |
| ATOM | 4314 | O | ILE | A 549 | 69.592 | 89.583 | −26.885 | 1.00 | 0.00 | O |
| ATOM | 4315 | CB | ILE | A 549 | 67.213 | 91.912 | −26.980 | 1.00 | 0.00 | C |
| ATOM | 4316 | CG1 | ILE | A 549 | 67.234 | 91.348 | −25.557 | 1.00 | 0.00 | C |
| ATOM | 4317 | CG2 | ILE | A 549 | 66.027 | 91.351 | −27.743 | 1.00 | 0.00 | C |
| ATOM | 4318 | CD1 | ILE | A 549 | 68.310 | 91.944 | −24.678 | 1.00 | 0.00 | C |
| ATOM | 4319 | N | LEU | A 550 | 68.034 | 89.310 | −28.477 | 1.00 | 0.00 | N |
| ATOM | 4320 | CA | LEU | A 550 | 68.152 | 87.853 | −28.478 | 1.00 | 0.00 | C |
| ATOM | 4321 | C | LEU | A 550 | 67.488 | 87.279 | −29.724 | 1.00 | 0.00 | C |
| ATOM | 4322 | O | LEU | A 550 | 66.284 | 87.430 | −29.923 | 1.00 | 0.00 | O |
| ATOM | 4323 | CB | LEU | A 550 | 67.479 | 87.283 | −27.226 | 1.00 | 0.00 | C |
| ATOM | 4324 | CG | LEU | A 550 | 67.571 | 85.771 | −27.006 | 1.00 | 0.00 | C |
| ATOM | 4325 | CD1 | LEU | A 550 | 69.021 | 85.384 | −26.764 | 1.00 | 0.00 | C |
| ATOM | 4326 | CD2 | LEU | A 550 | 66.715 | 85.372 | −25.817 | 1.00 | 0.00 | C |
| ATOM | 4327 | N | PRO | A 551 | 68.265 | 86.600 | −30.578 | 1.00 | 0.00 | N |
| ATOM | 4328 | CA | PRO | A 551 | 67.694 | 86.028 | −31.799 | 1.00 | 0.00 | C |
| ATOM | 4329 | C | PRO | A 551 | 66.889 | 84.732 | −31.671 | 1.00 | 0.00 | C |
| ATOM | 4330 | O | PRO | A 551 | 65.930 | 84.529 | −32.415 | 1.00 | 0.00 | O |
| ATOM | 4331 | CB | PRO | A 551 | 68.916 | 85.867 | −32.700 | 1.00 | 0.00 | C |
| ATOM | 4332 | CG | PRO | A 551 | 70.002 | 85.563 | −31.730 | 1.00 | 0.00 | C |
| ATOM | 4333 | CD | PRO | A 551 | 69.739 | 86.541 | −30.606 | 1.00 | 0.00 | C |
| ATOM | 4334 | N | SER | A 552 | 67.261 | 83.859 | −30.740 | 1.00 | 0.00 | N |
| ATOM | 4335 | CA | SER | A 552 | 66.543 | 82.594 | −30.601 | 1.00 | 0.00 | C |
| ATOM | 4336 | C | SER | A 552 | 66.366 | 82.135 | −29.161 | 1.00 | 0.00 | C |
| ATOM | 4337 | O | SER | A 552 | 66.993 | 82.659 | −28.243 | 1.00 | 0.00 | O |
| ATOM | 4338 | CB | SER | A 552 | 67.258 | 81.496 | −31.392 | 1.00 | 0.00 | C |
| ATOM | 4339 | OG | SER | A 552 | 68.563 | 81.285 | −30.886 | 1.00 | 0.00 | O |
| ATOM | 4340 | N | LYS | A 553 | 65.508 | 81.135 | −28.985 | 1.00 | 0.00 | N |
| ATOM | 4341 | CA | LYS | A 553 | 65.204 | 80.589 | −27.670 | 1.00 | 0.00 | C |
| ATOM | 4342 | C | LYS | A 553 | 64.897 | 79.092 | −27.735 | 1.00 | 0.00 | C |
| ATOM | 4343 | O | LYS | A 553 | 64.152 | 78.633 | −28.602 | 1.00 | 0.00 | O |
| ATOM | 4344 | CB | LYS | A 553 | 63.999 | 81.332 | −27.078 | 1.00 | 0.00 | C |
| ATOM | 4345 | CG | LYS | A 553 | 63.465 | 80.740 | −25.773 | 1.00 | 0.00 | C |
| ATOM | 4346 | CD | LYS | A 553 | 64.499 | 80.803 | −24.656 | 1.00 | 0.00 | C |
| ATOM | 4347 | CE | LYS | A 553 | 64.818 | 82.244 | −24.264 | 1.00 | 0.00 | C |
| ATOM | 4348 | NZ | LYS | A 553 | 65.931 | 82.304 | −23.268 | 1.00 | 0.00 | N |
| ATOM | 4349 | N | HIS | A 554 | 65.477 | 78.334 | −26.812 | 1.00 | 0.00 | N |
| ATOM | 4350 | CA | HIS | A 554 | 65.238 | 76.898 | −26.752 | 1.00 | 0.00 | C |
| ATOM | 4351 | C | HIS | A 554 | 64.046 | 76.596 | −25.857 | 1.00 | 0.00 | C |
| ATOM | 4352 | O | HIS | A 554 | 63.878 | 77.220 | −24.808 | 1.00 | 0.00 | O |
| ATOM | 4353 | CB | HIS | A 554 | 66.454 | 76.157 | −26.186 | 1.00 | 0.00 | C |
| ATOM | 4354 | CG | HIS | A 554 | 67.597 | 76.039 | −27.143 | 1.00 | 0.00 | C |
| ATOM | 4355 | ND1 | HIS | A 554 | 68.249 | 74.847 | −27.375 | 1.00 | 0.00 | N |
| ATOM | 4356 | CD2 | HIS | A 554 | 68.225 | 76.965 | −27.904 | 1.00 | 0.00 | C |
| ATOM | 4357 | CE1 | HIS | A 554 | 69.230 | 75.045 | −28.236 | 1.00 | 0.00 | C |
| ATOM | 4358 | NE2 | HIS | A 554 | 69.238 | 76.321 | −28.572 | 1.00 | 0.00 | N |
| ATOM | 4359 | N | VAL | A 555 | 63.227 | 75.640 | −26.286 | 1.00 | 0.00 | N |
| ATOM | 4360 | CA | VAL | A 555 | 62.066 | 75.192 | −25.530 | 1.00 | 0.00 | C |
| ATOM | 4361 | C | VAL | A 555 | 62.113 | 73.669 | −25.532 | 1.00 | 0.00 | C |
| ATOM | 4362 | O | VAL | A 555 | 62.600 | 73.059 | −26.490 | 1.00 | 0.00 | O |
| ATOM | 4363 | CB | VAL | A 555 | 60.729 | 75.651 | −26.160 | 1.00 | 0.00 | C |
| ATOM | 4364 | CG1 | VAL | A 555 | 60.615 | 77.168 | −26.092 | 1.00 | 0.00 | C |
| ATOM | 4365 | CG2 | VAL | A 555 | 60.620 | 75.156 | −27.591 | 1.00 | 0.00 | C |
| ATOM | 4366 | N | VAL | A 556 | 61.607 | 73.059 | −24.466 | 1.00 | 0.00 | N |
| ATOM | 4367 | CA | VAL | A 556 | 61.619 | 71.607 | −24.344 | 1.00 | 0.00 | C |
| ATOM | 4368 | C | VAL | A 556 | 60.260 | 71.052 | −23.925 | 1.00 | 0.00 | C |
| ATOM | 4369 | O | VAL | A 556 | 59.629 | 71.579 | −23.012 | 1.00 | 0.00 | O |
| ATOM | 4370 | CB | VAL | A 556 | 62.662 | 71.155 | −23.286 | 1.00 | 0.00 | C |
| ATOM | 4371 | CG1 | VAL | A 556 | 62.607 | 69.638 | −23.106 | 1.00 | 0.00 | C |
| ATOM | 4372 | CG2 | VAL | A 556 | 64.059 | 71.595 | −23.704 | 1.00 | 0.00 | C |
| ATOM | 4373 | N | MET | A 557 | 59.817 | 69.988 | −24.591 | 1.00 | 0.00 | N |
| ATOM | 4374 | CA | MET | A 557 | 58.551 | 69.343 | −24.244 | 1.00 | 0.00 | C |
| ATOM | 4375 | C | MET | A 557 | 58.803 | 67.991 | −23.576 | 1.00 | 0.00 | C |
| ATOM | 4376 | O | MET | A 557 | 59.709 | 67.253 | −23.966 | 1.00 | 0.00 | O |
| ATOM | 4377 | CB | MET | A 557 | 57.683 | 69.127 | −25.492 | 1.00 | 0.00 | C |
| ATOM | 4378 | CG | MET | A 557 | 56.649 | 70.216 | −25.760 | 1.00 | 0.00 | C |
| ATOM | 4379 | SD | MET | A 557 | 57.362 | 71.859 | −25.908 | 1.00 | 0.00 | S |
| ATOM | 4380 | CE | MET | A 557 | 58.355 | 71.676 | −27.418 | 1.00 | 0.00 | C |
| ATOM | 4381 | N | HIS | A 558 | 58.002 | 67.675 | −22.564 | 1.00 | 0.00 | N |
| ATOM | 4382 | CA | HIS | A 558 | 58.114 | 66.397 | −21.866 | 1.00 | 0.00 | C |
| ATOM | 4383 | C | HIS | A 558 | 56.828 | 65.609 | −22.085 | 1.00 | 0.00 | C |
| ATOM | 4384 | O | HIS | A 558 | 55.737 | 66.187 | −22.082 | 1.00 | 0.00 | O |
| ATOM | 4385 | CB | HIS | A 558 | 58.319 | 66.611 | −20.364 | 1.00 | 0.00 | C |
| ATOM | 4386 | CG | HIS | A 558 | 58.230 | 65.350 | −19.558 | 1.00 | 0.00 | C |
| ATOM | 4387 | ND1 | HIS | A 558 | 57.261 | 65.146 | −18.599 | 1.00 | 0.00 | N |
| ATOM | 4388 | CD2 | HIS | A 558 | 58.990 | 64.229 | −19.570 | 1.00 | 0.00 | C |
| ATOM | 4389 | CE1 | HIS | A 558 | 57.429 | 63.955 | −18.054 | 1.00 | 0.00 | C |
| ATOM | 4390 | NE2 | HIS | A 558 | 58.471 | 63.378 | −18.624 | 1.00 | 0.00 | N |
| ATOM | 4391 | N | ASN | A 559 | 56.963 | 64.299 | −22.286 | 1.00 | 0.00 | N |
| ATOM | 4392 | CA | ASN | A 559 | 55.820 | 63.414 | −22.497 | 1.00 | 0.00 | C |
| ATOM | 4393 | C | ASN | A 559 | 55.835 | 62.309 | −21.440 | 1.00 | 0.00 | C |
| ATOM | 4394 | O | ASN | A 559 | 56.575 | 61.333 | −21.554 | 1.00 | 0.00 | O |
| ATOM | 4395 | CB | ASN | A 559 | 55.878 | 62.807 | −23.904 | 1.00 | 0.00 | C |
| ATOM | 4396 | CG | ASN | A 559 | 54.865 | 61.691 | −24.102 | 1.00 | 0.00 | C |
| ATOM | 4397 | OD1 | ASN | A 559 | 53.762 | 61.735 | −23.558 | 1.00 | 0.00 | O |
| ATOM | 4398 | ND2 | ASN | A 559 | 55.233 | 60.690 | −24.899 | 1.00 | 0.00 | N |
| ATOM | 4399 | N | THR | A 560 | 55.010 | 62.459 | −20.411 | 1.00 | 0.00 | N |
| ATOM | 4400 | CA | THR | A 560 | 54.986 | 61.478 | −19.331 | 1.00 | 0.00 | C |
| ATOM | 4401 | C | THR | A 560 | 54.388 | 60.116 | −19.714 | 1.00 | 0.00 | C |
| ATOM | 4402 | O | THR | A 560 | 54.599 | 59.119 | −19.010 | 1.00 | 0.00 | O |
| ATOM | 4403 | CB | THR | A 560 | 54.241 | 62.050 | −18.106 | 1.00 | 0.00 | C |
| ATOM | 4404 | OG1 | THR | A 560 | 54.558 | 61.265 | −16.951 | 1.00 | 0.00 | O |
| ATOM | 4405 | CG2 | THR | A 560 | 52.733 | 62.046 | −18.340 | 1.00 | 0.00 | C |
| ATOM | 4406 | N | LEU | A 561 | 53.666 | 60.070 | −20.832 | 1.00 | 0.00 | N |
| ATOM | 4407 | CA | LEU | A 561 | 53.043 | 58.827 | −21.301 | 1.00 | 0.00 | C |
| ATOM | 4408 | C | LEU | A 561 | 54.070 | 57.891 | −21.953 | 1.00 | 0.00 | C |
| ATOM | 4409 | O | LEU | A 561 | 54.996 | 58.343 | −22.631 | 1.00 | 0.00 | O |
| ATOM | 4410 | CB | LEU | A 561 | 51.917 | 59.149 | −22.291 | 1.00 | 0.00 | C |
| ATOM | 4411 | CG | LEU | A 561 | 50.794 | 60.044 | −21.742 | 1.00 | 0.00 | C |
| ATOM | 4412 | CD1 | LEU | A 561 | 49.725 | 60.258 | −22.811 | 1.00 | 0.00 | C |
| ATOM | 4413 | CD2 | LEU | A 561 | 50.180 | 59.391 | −20.502 | 1.00 | 0.00 | C |
| ATOM | 4414 | N | PRO | A 562 | 53.908 | 56.568 | −21.758 | 1.00 | 0.00 | N |
| ATOM | 4415 | CA | PRO | A 562 | 54.821 | 55.562 | −22.312 | 1.00 | 0.00 | C |
| ATOM | 4416 | C | PRO | A 562 | 54.688 | 55.193 | −23.789 | 1.00 | 0.00 | C |
| ATOM | 4417 | O | PRO | A 562 | 54.857 | 54.030 | −24.159 | 1.00 | 0.00 | O |
| ATOM | 4418 | CB | PRO | A 562 | 54.604 | 54.367 | −21.390 | 1.00 | 0.00 | C |
| ATOM | 4419 | CG | PRO | A 562 | 53.138 | 54.455 | −21.110 | 1.00 | 0.00 | C |
| ATOM | 4420 | CD | PRO | A 562 | 52.921 | 55.938 | −20.861 | 1.00 | 0.00 | C |
| ATOM | 4421 | N | HIS | A 563 | 54.380 | 56.175 | −24.629 | 1.00 | 0.00 | N |
| ATOM | 4422 | CA | HIS | A 563 | 54.293 | 55.949 | −26.068 | 1.00 | 0.00 | C |
| ATOM | 4423 | C | HIS | A 563 | 54.628 | 57.246 | −26.787 | 1.00 | 0.00 | C |
| ATOM | 4424 | O | HIS | A 563 | 54.464 | 58.329 | −26.226 | 1.00 | 0.00 | O |
| ATOM | 4425 | CB | HIS | A 563 | 52.900 | 55.440 | −26.497 | 1.00 | 0.00 | C |
| ATOM | 4426 | CG | HIS | A 563 | 51.756 | 56.310 | −26.072 | 1.00 | 0.00 | C |
| ATOM | 4427 | ND1 | HIS | A 563 | 50.972 | 56.026 | −24.974 | 1.00 | 0.00 | N |
| ATOM | 4428 | CD2 | HIS | A 563 | 51.232 | 57.430 | −26.627 | 1.00 | 0.00 | C |
| ATOM | 4429 | CE1 | HIS | A 563 | 50.013 | 56.930 | −24.873 | 1.00 | 0.00 | C |
| ATOM | 4430 | NE2 | HIS | A 563 | 50.150 | 57.793 | −25.863 | 1.00 | 0.00 | N |
| ATOM | 4431 | N | TRP | A 564 | 55.134 | 57.140 | −28.010 | 1.00 | 0.00 | N |
| ATOM | 4432 | CA | TRP | A 564 | 55.460 | 58.335 | −28.772 | 1.00 | 0.00 | C |
| ATOM | 4433 | C | TRP | A 564 | 54.192 | 59.158 | −28.866 | 1.00 | 0.00 | C |
| ATOM | 4434 | O | TRP | A 564 | 53.106 | 58.612 | −29.049 | 1.00 | 0.00 | O |
| ATOM | 4435 | CB | TRP | A 564 | 55.935 | 57.978 | −30.182 | 1.00 | 0.00 | C |
| ATOM | 4436 | CG | TRP | A 564 | 57.368 | 57.567 | −30.238 | 1.00 | 0.00 | C |
| ATOM | 4437 | CD1 | TRP | A 564 | 57.868 | 56.309 | −30.089 | 1.00 | 0.00 | C |
| ATOM | 4438 | CD2 | TRP | A 564 | 58.493 | 58.433 | −30.410 | 1.00 | 0.00 | C |
| ATOM | 4439 | NE1 | TRP | A 564 | 59.242 | 56.335 | −30.155 | 1.00 | 0.00 | N |
| ATOM | 4440 | CE2 | TRP | A 564 | 59.651 | 57.628 | −30.351 | 1.00 | 0.00 | C |
| ATOM | 4441 | CE3 | TRP | A 564 | 58.636 | 59.815 | −30.606 | 1.00 | 0.00 | C |
| ATOM | 4442 | CZ2 | TRP | A 564 | 60.940 | 58.157 | −30.483 | 1.00 | 0.00 | C |
| ATOM | 4443 | CZ3 | TRP | A 564 | 59.920 | 60.343 | −30.737 | 1.00 | 0.00 | C |
| ATOM | 4444 | CH2 | TRP | A 564 | 61.054 | 59.513 | −30.675 | 1.00 | 0.00 | C |
| ATOM | 4445 | N | ARG | A 565 | 54.318 | 60.470 | −28.735 | 1.00 | 0.00 | N |
| ATOM | 4446 | CA | ARG | A 565 | 53.135 | 61.308 | −28.818 | 1.00 | 0.00 | C |
| ATOM | 4447 | C | ARG | A 565 | 53.384 | 62.625 | −29.520 | 1.00 | 0.00 | C |
| ATOM | 4448 | O | ARG | A 565 | 54.430 | 63.254 | −29.349 | 1.00 | 0.00 | O |
| ATOM | 4449 | CB | ARG | A 565 | 52.558 | 61.574 | −27.417 | 1.00 | 0.00 | C |
| ATOM | 4450 | CG | ARG | A 565 | 51.349 | 62.515 | −27.423 | 1.00 | 0.00 | C |
| ATOM | 4451 | CD | ARG | A 565 | 50.530 | 62.470 | −26.130 | 1.00 | 0.00 | C |
| ATOM | 4452 | NE | ARG | A 565 | 51.325 | 62.757 | −24.941 | 1.00 | 0.00 | N |
| ATOM | 4453 | CZ | ARG | A 565 | 50.820 | 63.218 | −23.799 | 1.00 | 0.00 | C |
| ATOM | 4454 | NH1 | ARG | A 565 | 49.514 | 63.454 | −23.691 | 1.00 | 0.00 | N |
| ATOM | 4455 | NH2 | ARG | A 565 | 51.618 | 63.440 | −22.764 | 1.00 | 0.00 | N |
| ATOM | 4456 | N | GLU | A 566 | 52.415 | 63.015 | −30.340 | 1.00 | 0.00 | N |
| ATOM | 4457 | CA | GLU | A 566 | 52.466 | 64.282 | −31.044 | 1.00 | 0.00 | C |
| ATOM | 4458 | C | GLU | A 566 | 51.327 | 65.098 | −30.457 | 1.00 | 0.00 | C |
| ATOM | 4459 | O | GLU | A 566 | 50.261 | 64.560 | −30.147 | 1.00 | 0.00 | O |
| ATOM | 4460 | CB | GLU | A 566 | 52.237 | 64.107 | −32.546 | 1.00 | 0.00 | C |
| ATOM | 4461 | CG | GLU | A 566 | 53.336 | 63.372 | −33.279 | 1.00 | 0.00 | C |
| ATOM | 4462 | CD | GLU | A 566 | 53.134 | 63.414 | −34.784 | 1.00 | 0.00 | C |
| ATOM | 4463 | OE1 | GLU | A 566 | 51.995 | 63.172 | −35.237 | 1.00 | 0.00 | O |
| ATOM | 4464 | OE2 | GLU | A 566 | 54.112 | 63.687 | −35.511 | 1.00 | 0.00 | O |
| ATOM | 4465 | N | GLN | A 567 | 51.560 | 66.392 | −30.289 | 1.00 | 0.00 | N |
| ATOM | 4466 | CA | GLN | A 567 | 50.551 | 67.284 | −29.741 | 1.00 | 0.00 | C |
| ATOM | 4467 | C | GLN | A 567 | 50.946 | 68.707 | −30.086 | 1.00 | 0.00 | C |
| ATOM | 4468 | O | GLN | A 567 | 52.131 | 69.053 | −30.060 | 1.00 | 0.00 | O |
| ATOM | 4469 | CB | GLN | A 567 | 50.472 | 67.148 | −28.214 | 1.00 | 0.00 | C |
| ATOM | 4470 | CG | GLN | A 567 | 49.464 | 68.103 | −27.572 | 1.00 | 0.00 | C |
| ATOM | 4471 | CD | GLN | A 567 | 49.787 | 68.434 | −26.122 | 1.00 | 0.00 | C |
| ATOM | 4472 | OE1 | GLN | A 567 | 49.685 | 67.586 | −25.235 | 1.00 | 0.00 | O |
| ATOM | 4473 | NE2 | GLN | A 567 | 50.186 | 69.679 | −25.880 | 1.00 | 0.00 | N |
| ATOM | 4474 | N | LEU | A 568 | 49.966 | 69.533 | −30.427 | 1.00 | 0.00 | N |
| ATOM | 4475 | CA | LEU | A 568 | 50.273 | 70.920 | −30.732 | 1.00 | 0.00 | C |
| ATOM | 4476 | C | LEU | A 568 | 50.583 | 71.615 | −29.412 | 1.00 | 0.00 | C |
| ATOM | 4477 | O | LEU | A 568 | 49.939 | 71.353 | −28.395 | 1.00 | 0.00 | O |
| ATOM | 4478 | CB | LEU | A 568 | 49.086 | 71.619 | −31.403 | 1.00 | 0.00 | C |
| ATOM | 4479 | CG | LEU | A 568 | 48.664 | 71.207 | −32.817 | 1.00 | 0.00 | C |
| ATOM | 4480 | CD1 | LEU | A 568 | 47.653 | 72.223 | −33.345 | 1.00 | 0.00 | C |
| ATOM | 4481 | CD2 | LEU | A 568 | 49.881 | 71.170 | −33.735 | 1.00 | 0.00 | C |
| ATOM | 4482 | N | VAL | A 569 | 51.585 | 72.485 | −29.429 | 1.00 | 0.00 | N |
| ATOM | 4483 | CA | VAL | A 569 | 51.966 | 73.246 | −28.248 | 1.00 | 0.00 | C |
| ATOM | 4484 | C | VAL | A 569 | 52.069 | 74.705 | −28.678 | 1.00 | 0.00 | C |
| ATOM | 4485 | O | VAL | A 569 | 52.361 | 74.990 | −29.836 | 1.00 | 0.00 | O |
| ATOM | 4486 | CB | VAL | A 569 | 53.331 | 72.778 | −27.683 | 1.00 | 0.00 | C |
| ATOM | 4487 | CG1 | VAL | A 569 | 53.198 | 71.370 | −27.099 | 1.00 | 0.00 | C |
| ATOM | 4488 | CG2 | VAL | A 569 | 54.385 | 72.797 | −28.781 | 1.00 | 0.00 | C |
| ATOM | 4489 | N | ASP | A 570 | 51.804 | 75.629 | −27.764 | 1.00 | 0.00 | N |
| ATOM | 4490 | CA | ASP | A 570 | 51.894 | 77.042 | −28.108 | 1.00 | 0.00 | C |
| ATOM | 4491 | C | ASP | A 570 | 52.699 | 77.818 | −27.077 | 1.00 | 0.00 | C |
| ATOM | 4492 | O | ASP | A 570 | 52.802 | 77.408 | −25.917 | 1.00 | 0.00 | O |
| ATOM | 4493 | CB | ASP | A 570 | 50.491 | 77.649 | −28.263 | 1.00 | 0.00 | C |
| ATOM | 4494 | CG | ASP | A 570 | 49.738 | 77.748 | −26.949 | 1.00 | 0.00 | C |
| ATOM | 4495 | OD1 | ASP | A 570 | 49.552 | 76.712 | −26.282 | 1.00 | 0.00 | O |
| ATOM | 4496 | OD2 | ASP | A 570 | 49.325 | 78.867 | −26.586 | 1.00 | 0.00 | O |
| ATOM | 4497 | N | PHE | A 571 | 53.303 | 78.917 | −27.522 | 1.00 | 0.00 | N |
| ATOM | 4498 | CA | PHE | A 571 | 54.097 | 79.785 | −26.656 | 1.00 | 0.00 | C |
| ATOM | 4499 | C | PHE | A 571 | 53.817 | 81.227 | −27.043 | 1.00 | 0.00 | C |
| ATOM | 4500 | O | PHE | A 571 | 53.464 | 81.504 | −28.187 | 1.00 | 0.00 | O |
| ATOM | 4501 | CB | PHE | A 571 | 55.602 | 79.557 | −26.841 | 1.00 | 0.00 | C |
| ATOM | 4502 | CG | PHE | A 571 | 56.070 | 78.184 | −26.475 | 1.00 | 0.00 | C |
| ATOM | 4503 | CD1 | PHE | A 571 | 56.026 | 77.151 | −27.403 | 1.00 | 0.00 | C |
| ATOM | 4504 | CD2 | PHE | A 571 | 56.568 | 77.927 | −25.202 | 1.00 | 0.00 | C |
| ATOM | 4505 | CE1 | PHE | A 571 | 56.477 | 75.876 | −27.068 | 1.00 | 0.00 | C |
| ATOM | 4506 | CE2 | PHE | A 571 | 57.020 | 76.652 | −24.858 | 1.00 | 0.00 | C |
| ATOM | 4507 | CZ | PHE | A 571 | 56.972 | 75.627 | −25.795 | 1.00 | 0.00 | C |
| ATOM | 4508 | N | TYR | A 572 | 53.975 | 82.141 | −26.091 | 1.00 | 0.00 | N |
| ATOM | 4509 | CA | TYR | A 572 | 53.794 | 83.560 | −26.376 | 1.00 | 0.00 | C |
| ATOM | 4510 | C | TYR | A 572 | 55.150 | 84.083 | −26.833 | 1.00 | 0.00 | C |
| ATOM | 4511 | O | TYR | A 572 | 56.167 | 83.773 | −26.214 | 1.00 | 0.00 | O |
| ATOM | 4512 | CB | TYR | A 572 | 53.376 | 84.336 | −25.122 | 1.00 | 0.00 | C |
| ATOM | 4513 | CG | TYR | A 572 | 51.940 | 84.150 | −24.683 | 1.00 | 0.00 | C |
| ATOM | 4514 | CD1 | TYR | A 572 | 51.027 | 83.448 | −25.468 | 1.00 | 0.00 | C |
| ATOM | 4515 | CD2 | TYR | A 572 | 51.488 | 84.706 | −23.485 | 1.00 | 0.00 | C |
| ATOM | 4516 | CE1 | TYR | A 572 | 49.698 | 83.304 | −25.073 | 1.00 | 0.00 | C |
| ATOM | 4517 | CE2 | TYR | A 572 | 50.161 | 84.570 | −23.082 | 1.00 | 0.00 | C |
| ATOM | 4518 | CZ | TYR | A 572 | 49.273 | 83.869 | −23.880 | 1.00 | 0.00 | C |
| ATOM | 4519 | OH | TYR | A 572 | 47.960 | 83.734 | −23.487 | 1.00 | 0.00 | O |
| ATOM | 4520 | N | VAL | A 573 | 55.167 | 84.864 | −27.911 | 1.00 | 0.00 | N |
| ATOM | 4521 | CA | VAL | A 573 | 56.406 | 85.443 | −28.433 | 1.00 | 0.00 | C |
| ATOM | 4522 | C | VAL | A 573 | 56.215 | 86.943 | −28.684 | 1.00 | 0.00 | C |
| ATOM | 4523 | O | VAL | A 573 | 55.105 | 87.393 | −28.950 | 1.00 | 0.00 | O |
| ATOM | 4524 | CB | VAL | A 573 | 56.848 | 84.757 | −29.751 | 1.00 | 0.00 | C |
| ATOM | 4525 | CG1 | VAL | A 573 | 57.301 | 83.327 | −29.469 | 1.00 | 0.00 | C |
| ATOM | 4526 | CG2 | VAL | A 573 | 55.703 | 84.759 | −30.750 | 1.00 | 0.00 | C |
| ATOM | 4527 | N | SER | A 574 | 57.299 | 87.709 | −28.599 | 1.00 | 0.00 | N |
| ATOM | 4528 | CA | SER | A 574 | 57.234 | 89.160 | −28.783 | 1.00 | 0.00 | C |
| ATOM | 4529 | C | SER | A 574 | 57.206 | 89.611 | −30.242 | 1.00 | 0.00 | C |
| ATOM | 4530 | O | SER | A 574 | 57.254 | 90.809 | −30.528 | 1.00 | 0.00 | O |
| ATOM | 4531 | CB | SER | A 574 | 58.413 | 89.828 | −28.070 | 1.00 | 0.00 | C |
| ATOM | 4532 | OG | SER | A 574 | 59.641 | 89.470 | −28.675 | 1.00 | 0.00 | O |
| ATOM | 4533 | N | SER | A 575 | 57.140 | 88.655 | −31.160 | 1.00 | 0.00 | N |
| ATOM | 4534 | CA | SER | A 575 | 57.090 | 88.967 | −32.582 | 1.00 | 0.00 | C |
| ATOM | 4535 | C | SER | A 575 | 56.345 | 87.885 | −33.340 | 1.00 | 0.00 | C |
| ATOM | 4536 | O | SER | A 575 | 56.420 | 86.708 | −32.992 | 1.00 | 0.00 | O |
| ATOM | 4537 | CB | SER | A 575 | 58.496 | 89.093 | −33.165 | 1.00 | 0.00 | C |
| ATOM | 4538 | OG | SER | A 575 | 58.437 | 89.188 | −34.581 | 1.00 | 0.00 | O |
| ATOM | 4539 | N | PRO | A 576 | 55.601 | 88.272 | −34.385 | 1.00 | 0.00 | N |
| ATOM | 4540 | CA | PRO | A 576 | 54.855 | 87.291 | −35.176 | 1.00 | 0.00 | C |
| ATOM | 4541 | C | PRO | A 576 | 55.781 | 86.604 | −36.177 | 1.00 | 0.00 | C |
| ATOM | 4542 | O | PRO | A 576 | 55.441 | 85.571 | −36.745 | 1.00 | 0.00 | O |
| ATOM | 4543 | CB | PRO | A 576 | 53.796 | 88.148 | −35.859 | 1.00 | 0.00 | C |
| ATOM | 4544 | CG | PRO | A 576 | 54.547 | 89.428 | −36.118 | 1.00 | 0.00 | C |
| ATOM | 4545 | CD | PRO | A 576 | 55.277 | 89.649 | −34.805 | 1.00 | 0.00 | C |
| ATOM | 4546 | N | PHE | A 577 | 56.960 | 87.187 | −36.379 | 1.00 | 0.00 | N |
| ATOM | 4547 | CA | PHE | A 577 | 57.936 | 86.652 | −37.321 | 1.00 | 0.00 | C |
| ATOM | 4548 | C | PHE | A 577 | 58.905 | 85.689 | −36.641 | 1.00 | 0.00 | C |
| ATOM | 4549 | O | PHE | A 577 | 60.086 | 85.993 | −36.459 | 1.00 | 0.00 | O |
| ATOM | 4550 | CB | PHE | A 577 | 58.701 | 87.806 | −37.967 | 1.00 | 0.00 | C |
| ATOM | 4551 | CG | PHE | A 577 | 57.812 | 88.798 | −38.663 | 1.00 | 0.00 | C |
| ATOM | 4552 | CD1 | PHE | A 577 | 58.065 | 90.163 | −38.571 | 1.00 | 0.00 | C |
| ATOM | 4553 | CD2 | PHE | A 577 | 56.716 | 88.368 | −39.406 | 1.00 | 0.00 | C |
| ATOM | 4554 | CE1 | PHE | A 577 | 57.237 | 91.088 | −39.208 | 1.00 | 0.00 | C |
| ATOM | 4555 | CE2 | PHE | A 577 | 55.881 | 89.284 | −40.049 | 1.00 | 0.00 | C |
| ATOM | 4556 | CZ | PHE | A 577 | 56.144 | 90.647 | −39.948 | 1.00 | 0.00 | C |
| ATOM | 4557 | N | VAL | A 578 | 58.384 | 84.521 | −36.278 | 1.00 | 0.00 | N |
| ATOM | 4558 | CA | VAL | A 578 | 59.166 | 83.496 | −35.603 | 1.00 | 0.00 | C |
| ATOM | 4559 | C | VAL | A 578 | 59.023 | 82.160 | −36.322 | 1.00 | 0.00 | C |
| ATOM | 4560 | O | VAL | A 578 | 57.937 | 81.804 | −36.779 | 1.00 | 0.00 | O |
| ATOM | 4561 | CB | VAL | A 578 | 58.700 | 83.332 | −34.133 | 1.00 | 0.00 | C |
| ATOM | 4562 | CG1 | VAL | A 578 | 59.511 | 82.245 | −33.435 | 1.00 | 0.00 | C |
| ATOM | 4563 | CG2 | VAL | A 578 | 58.839 | 84.659 | −33.399 | 1.00 | 0.00 | C |
| ATOM | 4564 | N | SER | A 579 | 60.126 | 81.428 | −36.427 | 1.00 | 0.00 | N |
| ATOM | 4565 | CA | SER | A 579 | 60.115 | 80.126 | −37.082 | 1.00 | 0.00 | C |
| ATOM | 4566 | C | SER | A 579 | 60.629 | 79.072 | −36.110 | 1.00 | 0.00 | C |
| ATOM | 4567 | O | SER | A 579 | 61.422 | 79.371 | −35.217 | 1.00 | 0.00 | O |
| ATOM | 4568 | CB | SER | A 579 | 60.976 | 80.152 | −38.351 | 1.00 | 0.00 | C |
| ATOM | 4569 | OG | SER | A 579 | 62.318 | 80.497 | −38.063 | 1.00 | 0.00 | O |
| ATOM | 4570 | N | VAL | A 580 | 60.174 | 77.839 | −36.292 | 1.00 | 0.00 | N |
| ATOM | 4571 | CA | VAL | A 580 | 60.556 | 76.739 | −35.421 | 1.00 | 0.00 | C |
| ATOM | 4572 | C | VAL | A 580 | 61.396 | 75.690 | −36.144 | 1.00 | 0.00 | C |
| ATOM | 4573 | O | VAL | A 580 | 61.190 | 75.418 | −37.327 | 1.00 | 0.00 | O |
| ATOM | 4574 | CB | VAL | A 580 | 59.298 | 76.042 | −34.849 | 1.00 | 0.00 | C |
| ATOM | 4575 | CG1 | VAL | A 580 | 59.695 | 74.989 | −33.819 | 1.00 | 0.00 | C |
| ATOM | 4576 | CG2 | VAL | A 580 | 58.369 | 77.073 | −34.235 | 1.00 | 0.00 | C |
| ATOM | 4577 | N | THR | A 581 | 62.342 | 75.111 | −35.412 | 1.00 | 0.00 | N |
| ATOM | 4578 | CA | THR | A 581 | 63.223 | 74.062 | −35.921 | 1.00 | 0.00 | C |
| ATOM | 4579 | C | THR | A 581 | 63.488 | 73.087 | −34.776 | 1.00 | 0.00 | C |
| ATOM | 4580 | O | THR | A 581 | 63.370 | 73.461 | −33.607 | 1.00 | 0.00 | O |
| ATOM | 4581 | CB | THR | A 581 | 64.591 | 74.626 | −36.372 | 1.00 | 0.00 | C |
| ATOM | 4582 | OG1 | THR | A 581 | 65.173 | 75.380 | −35.301 | 1.00 | 0.00 | O |
| ATOM | 4583 | CG2 | THR | A 581 | 64.438 | 75.519 | −37.604 | 1.00 | 0.00 | C |
| ATOM | 4584 | N | ASP | A 582 | 63.824 | 71.839 | −35.101 | 1.00 | 0.00 | N |
| ATOM | 4585 | CA | ASP | A 582 | 64.148 | 70.871 | −34.058 | 1.00 | 0.00 | C |
| ATOM | 4586 | C | ASP | A 582 | 65.656 | 70.994 | −33.839 | 1.00 | 0.00 | C |
| ATOM | 4587 | O | ASP | A 582 | 66.293 | 71.836 | −34.468 | 1.00 | 0.00 | O |
| ATOM | 4588 | CB | ASP | A 582 | 63.732 | 69.447 | −34.467 | 1.00 | 0.00 | C |
| ATOM | 4589 | CG | ASP | A 582 | 64.401 | 68.963 | −35.743 | 1.00 | 0.00 | C |
| ATOM | 4590 | OD1 | ASP | A 582 | 63.888 | 67.980 | −36.324 | 1.00 | 0.00 | O |
| ATOM | 4591 | OD2 | ASP | A 582 | 65.428 | 69.536 | −36.161 | 1.00 | 0.00 | O |
| ATOM | 4592 | N | LEU | A 583 | 66.250 | 70.184 | −32.970 | 1.00 | 0.00 | N |
| ATOM | 4593 | CA | LEU | A 583 | 67.681 | 70.357 | −32.750 | 1.00 | 0.00 | C |
| ATOM | 4594 | C | LEU | A 583 | 68.566 | 69.930 | −33.916 | 1.00 | 0.00 | C |
| ATOM | 4595 | O | LEU | A 583 | 69.761 | 70.219 | −33.922 | 1.00 | 0.00 | O |
| ATOM | 4596 | CB | LEU | A 583 | 68.141 | 69.660 | −31.470 | 1.00 | 0.00 | C |
| ATOM | 4597 | CG | LEU | A 583 | 69.404 | 70.352 | −30.945 | 1.00 | 0.00 | C |
| ATOM | 4598 | CD1 | LEU | A 583 | 69.062 | 71.782 | −30.548 | 1.00 | 0.00 | C |
| ATOM | 4599 | CD2 | LEU | A 583 | 69.976 | 69.605 | −29.768 | 1.00 | 0.00 | C |
| ATOM | 4600 | N | ALA | A 584 | 67.985 | 69.248 | −34.900 | 1.00 | 0.00 | N |
| ATOM | 4601 | CA | ALA | A 584 | 68.743 | 68.827 | −36.075 | 1.00 | 0.00 | C |
| ATOM | 4602 | C | ALA | A 584 | 68.633 | 69.945 | −37.107 | 1.00 | 0.00 | C |
| ATOM | 4603 | O | ALA | A 584 | 69.061 | 69.806 | −38.255 | 1.00 | 0.00 | O |
| ATOM | 4604 | CB | ALA | A 584 | 68.176 | 67.531 | −36.636 | 1.00 | 0.00 | C |
| ATOM | 4605 | N | ASN | A 585 | 68.049 | 71.057 | −36.673 | 1.00 | 0.00 | N |
| ATOM | 4606 | CA | ASN | A 585 | 67.854 | 72.227 | −37.516 | 1.00 | 0.00 | C |
| ATOM | 4607 | C | ASN | A 585 | 66.819 | 71.991 | −38.612 | 1.00 | 0.00 | C |
| ATOM | 4608 | O | ASN | A 585 | 66.787 | 72.720 | −39.603 | 1.00 | 0.00 | O |
| ATOM | 4609 | CB | ASN | A 585 | 69.178 | 72.663 | −38.149 | 1.00 | 0.00 | C |
| ATOM | 4610 | CG | ASN | A 585 | 69.481 | 74.127 | −37.907 | 1.00 | 0.00 | C |
| ATOM | 4611 | OD1 | ASN | A 585 | 68.607 | 74.988 | −38.041 | 1.00 | 0.00 | O |
| ATOM | 4612 | ND2 | ASN | A 585 | 70.726 | 74.420 | −37.554 | 1.00 | 0.00 | N |
| ATOM | 4613 | N | ASN | A 586 | 65.983 | 70.970 | −38.443 | 1.00 | 0.00 | N |
| ATOM | 4614 | CA | ASN | A 586 | 64.941 | 70.691 | −39.424 | 1.00 | 0.00 | C |
| ATOM | 4615 | C | ASN | A 586 | 63.801 | 71.669 | −39.183 | 1.00 | 0.00 | C |
| ATOM | 4616 | O | ASN | A 586 | 63.364 | 71.850 | −38.048 | 1.00 | 0.00 | O |
| ATOM | 4617 | CB | ASN | A 586 | 64.372 | 69.279 | −39.268 | 1.00 | 0.00 | C |
| ATOM | 4618 | CG | ASN | A 586 | 65.411 | 68.201 | −39.433 | 1.00 | 0.00 | C |
| ATOM | 4619 | OD1 | ASN | A 586 | 66.196 | 68.216 | −40.381 | 1.00 | 0.00 | O |
| ATOM | 4620 | ND2 | ASN | A 586 | 65.411 | 67.239 | −38.513 | 1.00 | 0.00 | N |
| ATOM | 4621 | N | PRO | A 587 | 63.302 | 72.313 | −40.244 | 1.00 | 0.00 | N |
| ATOM | 4622 | CA | PRO | A 587 | 62.197 | 73.254 | −40.045 | 1.00 | 0.00 | C |
| ATOM | 4623 | C | PRO | A 587 | 60.944 | 72.515 | −39.584 | 1.00 | 0.00 | C |
| ATOM | 4624 | O | PRO | A 587 | 60.730 | 71.353 | −39.933 | 1.00 | 0.00 | O |
| ATOM | 4625 | CB | PRO | A 587 | 62.034 | 73.895 | −41.423 | 1.00 | 0.00 | C |
| ATOM | 4626 | CG | PRO | A 587 | 62.496 | 72.817 | −42.358 | 1.00 | 0.00 | C |
| ATOM | 4627 | CD | PRO | A 587 | 63.716 | 72.271 | −41.658 | 1.00 | 0.00 | C |
| ATOM | 4628 | N | VAL | A 588 | 60.129 | 73.188 | −38.781 | 1.00 | 0.00 | N |
| ATOM | 4629 | CA | VAL | A 588 | 58.895 | 72.610 | −38.266 | 1.00 | 0.00 | C |
| ATOM | 4630 | C | VAL | A 588 | 57.776 | 73.579 | −38.621 | 1.00 | 0.00 | C |
| ATOM | 4631 | O | VAL | A 588 | 57.871 | 74.770 | −38.325 | 1.00 | 0.00 | O |
| ATOM | 4632 | CB | VAL | A 588 | 58.953 | 72.447 | −36.728 | 1.00 | 0.00 | C |
| ATOM | 4633 | CG1 | VAL | A 588 | 57.620 | 71.937 | −36.208 | 1.00 | 0.00 | C |
| ATOM | 4634 | CG2 | VAL | A 588 | 60.074 | 71.490 | −36.347 | 1.00 | 0.00 | C |
| ATOM | 4635 | N | GLU | A 589 | 56.725 | 73.079 | −39.264 | 1.00 | 0.00 | N |
| ATOM | 4636 | CA | GLU | A 589 | 55.621 | 73.946 | −39.649 | 1.00 | 0.00 | C |
| ATOM | 4637 | C | GLU | A 589 | 54.977 | 74.556 | −38.412 | 1.00 | 0.00 | C |
| ATOM | 4638 | O | GLU | A 589 | 54.704 | 73.859 | −37.435 | 1.00 | 0.00 | O |
| ATOM | 4639 | CB | GLU | A 589 | 54.567 | 73.175 | −40.441 | 1.00 | 0.00 | C |
| ATOM | 4640 | CG | GLU | A 589 | 53.664 | 74.090 | −41.253 | 1.00 | 0.00 | C |
| ATOM | 4641 | CD | GLU | A 589 | 52.443 | 73.385 | −41.799 | 1.00 | 0.00 | C |
| ATOM | 4642 | OE1 | GLU | A 589 | 52.573 | 72.226 | −42.248 | 1.00 | 0.00 | O |
| ATOM | 4643 | OE2 | GLU | A 589 | 51.351 | 73.998 | −41.787 | 1.00 | 0.00 | O |
| ATOM | 4644 | N | ALA | A 590 | 54.732 | 75.861 | −38.464 | 1.00 | 0.00 | N |
| ATOM | 4645 | CA | ALA | A 590 | 54.133 | 76.568 | −37.342 | 1.00 | 0.00 | C |
| ATOM | 4646 | C | ALA | A 590 | 53.039 | 77.519 | −37.805 | 1.00 | 0.00 | C |
| ATOM | 4647 | O | ALA | A 590 | 52.950 | 77.852 | −38.985 | 1.00 | 0.00 | O |
| ATOM | 4648 | CB | ALA | A 590 | 55.207 | 77.344 | −36.591 | 1.00 | 0.00 | C |
| ATOM | 4649 | N | GLN | A 591 | 52.209 | 77.943 | −36.860 | 1.00 | 0.00 | N |
| ATOM | 4650 | CA | GLN | A 591 | 51.118 | 78.874 | −37.125 | 1.00 | 0.00 | C |
| ATOM | 4651 | C | GLN | A 591 | 51.138 | 79.934 | −36.035 | 1.00 | 0.00 | C |
| ATOM | 4652 | O | GLN | A 591 | 51.301 | 79.618 | −34.855 | 1.00 | 0.00 | O |
| ATOM | 4653 | CB | GLN | A 591 | 49.764 | 78.157 | −37.079 | 1.00 | 0.00 | C |
| ATOM | 4654 | CG | GLN | A 591 | 48.561 | 79.103 | −37.149 | 1.00 | 0.00 | C |
| ATOM | 4655 | CD | GLN | A 591 | 47.235 | 78.403 | −36.881 | 1.00 | 0.00 | C |
| ATOM | 4656 | OE1 | GLN | A 591 | 46.958 | 77.339 | −37.437 | 1.00 | 0.00 | O |
| ATOM | 4657 | NE2 | GLN | A 591 | 46.407 | 79.004 | −36.034 | 1.00 | 0.00 | N |
| ATOM | 4658 | N | VAL | A 592 | 50.987 | 81.193 | −36.425 | 1.00 | 0.00 | N |
| ATOM | 4659 | CA | VAL | A 592 | 50.958 | 82.266 | −35.446 | 1.00 | 0.00 | C |
| ATOM | 4660 | C | VAL | A 592 | 49.550 | 82.840 | −35.410 | 1.00 | 0.00 | C |
| ATOM | 4661 | O | VAL | A 592 | 48.915 | 83.026 | −36.447 | 1.00 | 0.00 | O |
| ATOM | 4662 | CB | VAL | A 592 | 51.973 | 83.381 | −35.782 | 1.00 | 0.00 | C |
| ATOM | 4663 | CG1 | VAL | A 592 | 51.754 | 84.583 | −34.861 | 1.00 | 0.00 | C |
| ATOM | 4664 | CG2 | VAL | A 592 | 53.395 | 82.851 | −35.609 | 1.00 | 0.00 | C |
| ATOM | 4665 | N | SER | A 593 | 49.057 | 83.082 | −34.203 | 1.00 | 0.00 | N |
| ATOM | 4666 | CA | SER | A 593 | 47.729 | 83.643 | −34.002 | 1.00 | 0.00 | C |
| ATOM | 4667 | C | SER | A 593 | 47.867 | 84.779 | −33.003 | 1.00 | 0.00 | C |
| ATOM | 4668 | O | SER | A 593 | 48.873 | 84.879 | −32.296 | 1.00 | 0.00 | O |
| ATOM | 4669 | CB | SER | A 593 | 46.768 | 82.586 | −33.436 | 1.00 | 0.00 | C |
| ATOM | 4670 | OG | SER | A 593 | 46.601 | 81.492 | −34.326 | 1.00 | 0.00 | O |
| ATOM | 4671 | N | PRO | A 594 | 46.862 | 85.660 | −32.932 | 1.00 | 0.00 | N |
| ATOM | 4672 | CA | PRO | A 594 | 46.940 | 86.773 | −31.983 | 1.00 | 0.00 | C |
| ATOM | 4673 | C | PRO | A 594 | 46.674 | 86.288 | −30.563 | 1.00 | 0.00 | C |
| ATOM | 4674 | O | PRO | A 594 | 46.236 | 85.153 | −30.354 | 1.00 | 0.00 | O |
| ATOM | 4675 | CB | PRO | A 594 | 45.832 | 87.716 | −32.453 | 1.00 | 0.00 | C |
| ATOM | 4676 | CG | PRO | A 594 | 45.611 | 87.340 | −33.899 | 1.00 | 0.00 | C |
| ATOM | 4677 | CD | PRO | A 594 | 45.739 | 85.842 | −33.866 | 1.00 | 0.00 | C |
| ATOM | 4678 | N | VAL | A 595 | 46.951 | 87.146 | −29.589 | 1.00 | 0.00 | N |
| ATOM | 4679 | CA | VAL | A 595 | 46.669 | 86.819 | −28.201 | 1.00 | 0.00 | C |
| ATOM | 4680 | C | VAL | A 595 | 45.381 | 87.572 | −27.896 | 1.00 | 0.00 | C |
| ATOM | 4681 | O | VAL | A 595 | 45.387 | 88.794 | −27.762 | 1.00 | 0.00 | O |
| ATOM | 4682 | CB | VAL | A 595 | 47.778 | 87.302 | −27.244 | 1.00 | 0.00 | C |
| ATOM | 4683 | CG1 | VAL | A 595 | 47.342 | 87.080 | −25.795 | 1.00 | 0.00 | C |
| ATOM | 4684 | CG2 | VAL | A 595 | 49.065 | 86.542 | −27.518 | 1.00 | 0.00 | C |
| ATOM | 4685 | N | TRP | A 596 | 44.274 | 86.840 | −27.812 | 1.00 | 0.00 | N |
| ATOM | 4686 | CA | TRP | A 596 | 42.977 | 87.443 | −27.543 | 1.00 | 0.00 | C |
| ATOM | 4687 | C | TRP | A 596 | 42.569 | 87.333 | −26.079 | 1.00 | 0.00 | C |
| ATOM | 4688 | O | TRP | A 596 | 42.694 | 86.272 | −25.466 | 1.00 | 0.00 | O |
| ATOM | 4689 | CB | TRP | A 596 | 41.886 | 86.783 | −28.400 | 1.00 | 0.00 | C |
| ATOM | 4690 | CG | TRP | A 596 | 42.049 | 86.945 | −29.879 | 1.00 | 0.00 | C |
| ATOM | 4691 | CD1 | TRP | A 596 | 42.507 | 86.009 | −30.764 | 1.00 | 0.00 | C |
| ATOM | 4692 | CD2 | TRP | A 596 | 41.740 | 88.111 | −30.652 | 1.00 | 0.00 | C |
| ATOM | 4693 | NE1 | TRP | A 596 | 42.498 | 86.519 | −32.041 | 1.00 | 0.00 | N |
| ATOM | 4694 | CE2 | TRP | A 596 | 42.033 | 87.808 | −32.000 | 1.00 | 0.00 | C |
| ATOM | 4695 | CE3 | TRP | A 596 | 41.242 | 89.383 | −30.335 | 1.00 | 0.00 | C |
| ATOM | 4696 | CZ2 | TRP | A 596 | 41.845 | 88.732 | −33.034 | 1.00 | 0.00 | C |
| ATOM | 4697 | CZ3 | TRP | A 596 | 41.054 | 90.305 | −31.366 | 1.00 | 0.00 | C |
| ATOM | 4698 | CH2 | TRP | A 596 | 41.356 | 89.971 | −32.699 | 1.00 | 0.00 | C |
| ATOM | 4699 | N | SER | A 597 | 42.079 | 88.436 | −25.524 | 1.00 | 0.00 | N |
| ATOM | 4700 | CA | SER | A 597 | 41.618 | 88.455 | −24.142 | 1.00 | 0.00 | C |
| ATOM | 4701 | C | SER | A 597 | 40.221 | 89.063 | −24.128 | 1.00 | 0.00 | C |
| ATOM | 4702 | O | SER | A 597 | 39.975 | 90.094 | −24.758 | 1.00 | 0.00 | O |
| ATOM | 4703 | CB | SER | A 597 | 42.563 | 89.281 | −23.267 | 1.00 | 0.00 | C |
| ATOM | 4704 | OG | SER | A 597 | 42.711 | 90.586 | −23.785 | 1.00 | 0.00 | O |
| ATOM | 4705 | N | TRP | A 598 | 39.305 | 88.414 | −23.420 | 1.00 | 0.00 | N |
| ATOM | 4706 | CA | TRP | A 598 | 37.933 | 88.890 | −23.344 | 1.00 | 0.00 | C |
| ATOM | 4707 | C | TRP | A 598 | 37.721 | 89.802 | −22.148 | 1.00 | 0.00 | C |
| ATOM | 4708 | O | TRP | A 598 | 38.207 | 89.532 | −21.050 | 1.00 | 0.00 | O |
| ATOM | 4709 | CB | TRP | A 598 | 36.968 | 87.705 | −23.289 | 1.00 | 0.00 | C |
| ATOM | 4710 | CG | TRP | A 598 | 36.941 | 86.932 | −24.571 | 1.00 | 0.00 | C |
| ATOM | 4711 | CD1 | TRP | A 598 | 37.903 | 86.082 | −25.040 | 1.00 | 0.00 | C |
| ATOM | 4712 | CD2 | TRP | A 598 | 35.923 | 86.978 | −25.575 | 1.00 | 0.00 | C |
| ATOM | 4713 | NE1 | TRP | A 598 | 37.544 | 85.595 | −26.277 | 1.00 | 0.00 | N |
| ATOM | 4714 | CE2 | TRP | A 598 | 36.334 | 86.130 | −26.629 | 1.00 | 0.00 | C |
| ATOM | 4715 | CE3 | TRP | A 598 | 34.701 | 87.657 | −25.688 | 1.00 | 0.00 | C |
| ATOM | 4716 | CZ2 | TRP | A 598 | 35.566 | 85.942 | −27.782 | 1.00 | 0.00 | C |
| ATOM | 4717 | CZ3 | TRP | A 598 | 33.938 | 87.470 | −26.838 | 1.00 | 0.00 | C |
| ATOM | 4718 | CH2 | TRP | A 598 | 34.375 | 86.619 | −27.869 | 1.00 | 0.00 | C |
| ATOM | 4719 | N | HIS | A 599 | 36.988 | 90.885 | −22.368 | 1.00 | 0.00 | N |
| ATOM | 4720 | CA | HIS | A 599 | 36.737 | 91.849 | −21.311 | 1.00 | 0.00 | C |
| ATOM | 4721 | C | HIS | A 599 | 35.281 | 92.272 | −21.237 | 1.00 | 0.00 | C |
| ATOM | 4722 | O | HIS | A 599 | 34.620 | 92.452 | −22.261 | 1.00 | 0.00 | O |
| ATOM | 4723 | CB | HIS | A 599 | 37.618 | 93.079 | −21.530 | 1.00 | 0.00 | C |
| ATOM | 4724 | CG | HIS | A 599 | 39.080 | 92.764 | −21.595 | 1.00 | 0.00 | C |
| ATOM | 4725 | ND1 | HIS | A 599 | 39.807 | 92.371 | −20.492 | 1.00 | 0.00 | N |
| ATOM | 4726 | CD2 | HIS | A 599 | 39.942 | 92.749 | −22.639 | 1.00 | 0.00 | C |
| ATOM | 4727 | CE1 | HIS | A 599 | 41.054 | 92.128 | −20.854 | 1.00 | 0.00 | C |
| ATOM | 4728 | NE2 | HIS | A 599 | 41.162 | 92.349 | −22.152 | 1.00 | 0.00 | N |
| ATOM | 4729 | N | HIS | A 600 | 34.785 | 92.418 | −20.015 | 1.00 | 0.00 | N |
| ATOM | 4730 | CA | HIS | A 600 | 33.413 | 92.846 | −19.800 | 1.00 | 0.00 | C |
| ATOM | 4731 | C | HIS | A 600 | 33.458 | 94.366 | −19.818 | 1.00 | 0.00 | C |
| ATOM | 4732 | O | HIS | A 600 | 33.805 | 94.997 | −18.820 | 1.00 | 0.00 | O |
| ATOM | 4733 | CB | HIS | A 600 | 32.901 | 92.352 | −18.446 | 1.00 | 0.00 | C |
| ATOM | 4734 | CG | HIS | A 600 | 31.441 | 92.602 | −18.225 | 1.00 | 0.00 | C |
| ATOM | 4735 | ND1 | HIS | A 600 | 30.866 | 93.845 | −18.386 | 1.00 | 0.00 | N |
| ATOM | 4736 | CD2 | HIS | A 600 | 30.440 | 91.770 | −17.853 | 1.00 | 0.00 | C |
| ATOM | 4737 | CE1 | HIS | A 600 | 29.573 | 93.768 | −18.123 | 1.00 | 0.00 | C |
| ATOM | 4738 | NE2 | HIS | A 600 | 29.289 | 92.519 | −17.797 | 1.00 | 0.00 | N |
| ATOM | 4739 | N | ASP | A 601 | 33.126 | 94.947 | −20.965 | 1.00 | 0.00 | N |
| ATOM | 4740 | CA | ASP | A 601 | 33.146 | 96.394 | −21.123 | 1.00 | 0.00 | C |
| ATOM | 4741 | C | ASP | A 601 | 32.061 | 97.056 | −20.278 | 1.00 | 0.00 | C |
| ATOM | 4742 | O | ASP | A 601 | 30.879 | 97.001 | −20.615 | 1.00 | 0.00 | O |
| ATOM | 4743 | CB | ASP | A 601 | 32.953 | 96.760 | −22.595 | 1.00 | 0.00 | C |
| ATOM | 4744 | CG | ASP | A 601 | 33.347 | 98.191 | −22.896 | 1.00 | 0.00 | C |
| ATOM | 4745 | OD1 | ASP | A 601 | 32.849 | 99.108 | −22.211 | 1.00 | 0.00 | O |
| ATOM | 4746 | OD2 | ASP | A 601 | 34.155 | 98.399 | −23.824 | 1.00 | 0.00 | O |
| ATOM | 4747 | N | THR | A 602 | 32.475 | 97.683 | −19.180 | 1.00 | 0.00 | N |
| ATOM | 4748 | CA | THR | A 602 | 31.550 | 98.358 | −18.276 | 1.00 | 0.00 | C |
| ATOM | 4749 | C | THR | A 602 | 30.828 | 99.511 | −18.964 | 1.00 | 0.00 | C |
| ATOM | 4750 | O | THR | A 602 | 29.728 | 99.890 | −18.561 | 1.00 | 0.00 | O |
| ATOM | 4751 | CB | THR | A 602 | 32.288 | 98.921 | −17.040 | 1.00 | 0.00 | C |
| ATOM | 4752 | OG1 | THR | A 602 | 32.931 | 97.852 | −16.337 | 1.00 | 0.00 | O |
| ATOM | 4753 | CG2 | THR | A 602 | 31.307 | 99.619 | −16.101 | 1.00 | 0.00 | C |
| ATOM | 4754 | N | LEU | A 603 | 31.448 | 100.062 | −20.003 | 1.00 | 0.00 | N |
| ATOM | 4755 | CA | LEU | A 603 | 30.868 | 101.183 | −20.735 | 1.00 | 0.00 | C |
| ATOM | 4756 | C | LEU | A 603 | 29.841 | 100.767 | −21.784 | 1.00 | 0.00 | C |
| ATOM | 4757 | O | LEU | A 603 | 28.697 | 101.217 | −21.752 | 1.00 | 0.00 | O |
| ATOM | 4758 | CB | LEU | A 603 | 31.973 | 102.008 | −21.407 | 1.00 | 0.00 | C |
| ATOM | 4759 | CG | LEU | A 603 | 32.940 | 102.781 | −20.504 | 1.00 | 0.00 | C |
| ATOM | 4760 | CD1 | LEU | A 603 | 32.150 | 103.721 | −19.603 | 1.00 | 0.00 | C |
| ATOM | 4761 | CD2 | LEU | A 603 | 33.769 | 101.815 | −19.671 | 1.00 | 0.00 | C |
| ATOM | 4762 | N | THR | A 604 | 30.253 | 99.913 | −22.714 | 1.00 | 0.00 | N |
| ATOM | 4763 | CA | THR | A 604 | 29.363 | 99.455 | −23.776 | 1.00 | 0.00 | C |
| ATOM | 4764 | C | THR | A 604 | 28.457 | 98.314 | −23.328 | 1.00 | 0.00 | C |
| ATOM | 4765 | O | THR | A 604 | 27.566 | 97.895 | −24.066 | 1.00 | 0.00 | O |
| ATOM | 4766 | CB | THR | A 604 | 30.162 | 98.980 | −24.999 | 1.00 | 0.00 | C |
| ATOM | 4767 | OG1 | THR | A 604 | 30.974 | 97.858 | −24.630 | 1.00 | 0.00 | O |
| ATOM | 4768 | CG2 | THR | A 604 | 31.054 | 100.100 | −25.520 | 1.00 | 0.00 | C |
| ATOM | 4769 | N | LYS | A 605 | 28.689 | 97.814 | −22.120 | 1.00 | 0.00 | N |
| ATOM | 4770 | CA | LYS | A 605 | 27.892 | 96.717 | −21.582 | 1.00 | 0.00 | C |
| ATOM | 4771 | C | LYS | A 605 | 27.894 | 95.503 | −22.511 | 1.00 | 0.00 | C |
| ATOM | 4772 | O | LYS | A 605 | 26.865 | 94.858 | −22.703 | 1.00 | 0.00 | O |
| ATOM | 4773 | CB | LYS | A 605 | 26.452 | 97.178 | −21.343 | 1.00 | 0.00 | C |
| ATOM | 4774 | CG | LYS | A 605 | 26.328 | 98.317 | −20.345 | 1.00 | 0.00 | C |
| ATOM | 4775 | CD | LYS | A 605 | 26.799 | 97.897 | −18.959 | 1.00 | 0.00 | C |
| ATOM | 4776 | CE | LYS | A 605 | 26.693 | 99.051 | −17.972 | 1.00 | 0.00 | C |
| ATOM | 4777 | NZ | LYS | A 605 | 27.086 | 98.649 | −16.595 | 1.00 | 0.00 | N |
| ATOM | 4778 | N | THR | A 606 | 29.053 | 95.204 | −23.090 | 1.00 | 0.00 | N |
| ATOM | 4779 | CA | THR | A 606 | 29.198 | 94.060 | −23.986 | 1.00 | 0.00 | C |
| ATOM | 4780 | C | THR | A 606 | 30.454 | 93.289 | −23.607 | 1.00 | 0.00 | C |
| ATOM | 4781 | O | THR | A 606 | 31.371 | 93.841 | −23.004 | 1.00 | 0.00 | O |
| ATOM | 4782 | CB | THR | A 606 | 29.340 | 94.496 | −25.461 | 1.00 | 0.00 | C |
| ATOM | 4783 | OG1 | THR | A 606 | 30.503 | 95.325 | −25.606 | 1.00 | 0.00 | O |
| ATOM | 4784 | CG2 | THR | A 606 | 28.109 | 95.264 | −25.915 | 1.00 | 0.00 | C |
| ATOM | 4785 | N | ILE | A 607 | 30.484 | 92.006 | −23.947 | 1.00 | 0.00 | N |
| ATOM | 4786 | CA | ILE | A 607 | 31.645 | 91.173 | −23.666 | 1.00 | 0.00 | C |
| ATOM | 4787 | C | ILE | A 607 | 32.333 | 90.955 | −25.009 | 1.00 | 0.00 | C |
| ATOM | 4788 | O | ILE | A 607 | 31.806 | 90.263 | −25.880 | 1.00 | 0.00 | O |
| ATOM | 4789 | CB | ILE | A 607 | 31.226 | 89.819 | −23.069 | 1.00 | 0.00 | C |
| ATOM | 4790 | CG1 | ILE | A 607 | 30.416 | 90.047 | −21.787 | 1.00 | 0.00 | C |
| ATOM | 4791 | CG2 | ILE | A 607 | 32.461 | 88.981 | −22.769 | 1.00 | 0.00 | C |
| ATOM | 4792 | CD1 | ILE | A 607 | 29.720 | 88.811 | −21.267 | 1.00 | 0.00 | C |
| ATOM | 4793 | N | HIS | A 608 | 33.505 | 91.560 | −25.186 | 1.00 | 0.00 | N |
| ATOM | 4794 | CA | HIS | A 608 | 34.221 | 91.434 | −26.449 | 1.00 | 0.00 | C |
| ATOM | 4795 | C | HIS | A 608 | 35.720 | 91.224 | −26.272 | 1.00 | 0.00 | C |
| ATOM | 4796 | O | HIS | A 608 | 36.291 | 91.545 | −25.227 | 1.00 | 0.00 | O |
| ATOM | 4797 | CB | HIS | A 608 | 33.965 | 92.665 | −27.321 | 1.00 | 0.00 | C |
| ATOM | 4798 | CG | HIS | A 608 | 34.490 | 93.938 | −26.738 | 1.00 | 0.00 | C |
| ATOM | 4799 | ND1 | HIS | A 608 | 34.011 | 94.472 | −25.562 | 1.00 | 0.00 | N |
| ATOM | 4800 | CD2 | HIS | A 608 | 35.455 | 94.784 | −27.171 | 1.00 | 0.00 | C |
| ATOM | 4801 | CE1 | HIS | A 608 | 34.657 | 95.594 | −25.295 | 1.00 | 0.00 | C |
| ATOM | 4802 | NE2 | HIS | A 608 | 35.538 | 95.806 | −26.256 | 1.00 | 0.00 | N |
| ATOM | 4803 | N | PRO | A 609 | 36.380 | 90.685 | −27.307 | 1.00 | 0.00 | N |
| ATOM | 4804 | CA | PRO | A 609 | 37.818 | 90.425 | −27.259 | 1.00 | 0.00 | C |
| ATOM | 4805 | C | PRO | A 609 | 38.709 | 91.561 | −27.733 | 1.00 | 0.00 | C |
| ATOM | 4806 | O | PRO | A 609 | 38.363 | 92.302 | −28.654 | 1.00 | 0.00 | O |
| ATOM | 4807 | CB | PRO | A 609 | 37.958 | 89.196 | −28.141 | 1.00 | 0.00 | C |
| ATOM | 4808 | CG | PRO | A 609 | 36.996 | 89.518 | −29.251 | 1.00 | 0.00 | C |
| ATOM | 4809 | CD | PRO | A 609 | 35.784 | 90.098 | −28.524 | 1.00 | 0.00 | C |
| ATOM | 4810 | N | GLN | A 610 | 39.862 | 91.684 | −27.085 | 1.00 | 0.00 | N |
| ATOM | 4811 | CA | GLN | A 610 | 40.854 | 92.688 | −27.438 | 1.00 | 0.00 | C |
| ATOM | 4812 | C | GLN | A 610 | 42.117 | 91.913 | −27.788 | 1.00 | 0.00 | C |
| ATOM | 4813 | O | GLN | A 610 | 42.469 | 90.953 | −27.103 | 1.00 | 0.00 | O |
| ATOM | 4814 | CB | GLN | A 610 | 41.138 | 93.626 | −26.259 | 1.00 | 0.00 | C |
| ATOM | 4815 | CG | GLN | A 610 | 39.957 | 94.489 | −25.829 | 1.00 | 0.00 | C |
| ATOM | 4816 | CD | GLN | A 610 | 40.344 | 95.515 | −24.773 | 1.00 | 0.00 | C |
| ATOM | 4817 | OE1 | GLN | A 610 | 40.839 | 95.165 | −23.699 | 1.00 | 0.00 | O |
| ATOM | 4818 | NE2 | GLN | A 610 | 40.122 | 96.790 | −25.077 | 1.00 | 0.00 | N |
| ATOM | 4819 | N | GLY | A 611 | 42.787 | 92.319 | −28.859 | 1.00 | 0.00 | N |
| ATOM | 4820 | CA | GLY | A 611 | 44.000 | 91.634 | −29.262 | 1.00 | 0.00 | C |
| ATOM | 4821 | C | GLY | A 611 | 45.252 | 92.383 | −28.852 | 1.00 | 0.00 | C |
| ATOM | 4822 | O | GLY | A 611 | 45.276 | 93.615 | −28.848 | 1.00 | 0.00 | O |
| ATOM | 4823 | N | SER | A 612 | 46.294 | 91.639 | −28.496 | 1.00 | 0.00 | N |
| ATOM | 4824 | CA | SER | A 612 | 47.559 | 92.244 | −28.093 | 1.00 | 0.00 | C |
| ATOM | 4825 | C | SER | A 612 | 48.306 | 92.772 | −29.311 | 1.00 | 0.00 | C |
| ATOM | 4826 | O | SER | A 612 | 48.214 | 92.201 | −30.396 | 1.00 | 0.00 | O |
| ATOM | 4827 | CB | SER | A 612 | 48.437 | 91.215 | −27.379 | 1.00 | 0.00 | C |
| ATOM | 4828 | OG | SER | A 612 | 49.742 | 91.729 | −27.169 | 1.00 | 0.00 | O |
| ATOM | 4829 | N | THR | A 613 | 49.047 | 93.862 | −29.126 | 1.00 | 0.00 | N |
| ATOM | 4830 | CA | THR | A 613 | 49.822 | 94.450 | −30.214 | 1.00 | 0.00 | C |
| ATOM | 4831 | C | THR | A 613 | 51.321 | 94.258 | −29.975 | 1.00 | 0.00 | C |
| ATOM | 4832 | O | THR | A 613 | 52.145 | 94.760 | −30.741 | 1.00 | 0.00 | O |
| ATOM | 4833 | CB | THR | A 613 | 49.550 | 95.962 | −30.358 | 1.00 | 0.00 | C |
| ATOM | 4834 | OG1 | THR | A 613 | 49.909 | 96.628 | −29.141 | 1.00 | 0.00 | O |
| ATOM | 4835 | CG2 | THR | A 613 | 48.076 | 96.216 | −30.661 | 1.00 | 0.00 | C |
| ATOM | 4836 | N | THR | A 614 | 51.665 | 93.522 | −28.919 | 1.00 | 0.00 | N |
| ATOM | 4837 | CA | THR | A 614 | 53.065 | 93.272 | −28.570 | 1.00 | 0.00 | C |
| ATOM | 4838 | C | THR | A 614 | 53.371 | 91.799 | −28.288 | 1.00 | 0.00 | C |
| ATOM | 4839 | O | THR | A 614 | 54.535 | 91.405 | −28.190 | 1.00 | 0.00 | O |
| ATOM | 4840 | CB | THR | A 614 | 53.469 | 94.067 | −27.318 | 1.00 | 0.00 | C |
| ATOM | 4841 | OG1 | THR | A 614 | 52.573 | 93.750 | −26.244 | 1.00 | 0.00 | O |
| ATOM | 4842 | CG2 | THR | A 614 | 53.421 | 95.562 | −27.592 | 1.00 | 0.00 | C |
| ATOM | 4843 | N | LYS | A 615 | 52.324 | 90.993 | −28.158 | 1.00 | 0.00 | N |
| ATOM | 4844 | CA | LYS | A 615 | 52.468 | 89.574 | −27.856 | 1.00 | 0.00 | C |
| ATOM | 4845 | C | LYS | A 615 | 51.682 | 88.728 | −28.858 | 1.00 | 0.00 | C |
| ATOM | 4846 | O | LYS | A 615 | 50.582 | 89.108 | −29.265 | 1.00 | 0.00 | O |
| ATOM | 4847 | CB | LYS | A 615 | 51.952 | 89.324 | −26.435 | 1.00 | 0.00 | C |
| ATOM | 4848 | CG | LYS | A 615 | 51.865 | 87.874 | −26.015 | 1.00 | 0.00 | C |
| ATOM | 4849 | CD | LYS | A 615 | 51.022 | 87.725 | −24.746 | 1.00 | 0.00 | C |
| ATOM | 4850 | CE | LYS | A 615 | 51.586 | 88.531 | −23.586 | 1.00 | 0.00 | C |
| ATOM | 4851 | NZ | LYS | A 615 | 50.784 | 88.342 | −22.340 | 1.00 | 0.00 | N |
| ATOM | 4852 | N | TYR | A 616 | 52.248 | 87.589 | −29.257 | 1.00 | 0.00 | N |
| ATOM | 4853 | CA | TYR | A 616 | 51.589 | 86.695 | −30.211 | 1.00 | 0.00 | C |
| ATOM | 4854 | C | TYR | A 616 | 51.764 | 85.230 | −29.823 | 1.00 | 0.00 | C |
| ATOM | 4855 | O | TYR | A 616 | 52.678 | 84.888 | −29.078 | 1.00 | 0.00 | O |
| ATOM | 4856 | CB | TYR | A 616 | 52.145 | 86.921 | −31.621 | 1.00 | 0.00 | C |
| ATOM | 4857 | CG | TYR | A 616 | 52.153 | 88.376 | −32.015 | 1.00 | 0.00 | C |
| ATOM | 4858 | CD1 | TYR | A 616 | 53.238 | 89.192 | −31.700 | 1.00 | 0.00 | C |
| ATOM | 4859 | CD2 | TYR | A 616 | 51.041 | 88.958 | −32.624 | 1.00 | 0.00 | C |
| ATOM | 4860 | CE1 | TYR | A 616 | 53.217 | 90.552 | −31.973 | 1.00 | 0.00 | C |
| ATOM | 4861 | CE2 | TYR | A 616 | 51.008 | 90.323 | −32.901 | 1.00 | 0.00 | C |
| ATOM | 4862 | CZ | TYR | A 616 | 52.099 | 91.112 | −32.570 | 1.00 | 0.00 | C |
| ATOM | 4863 | OH | TYR | A 616 | 52.072 | 92.463 | −32.826 | 1.00 | 0.00 | O |
| ATOM | 4864 | N | ARG | A 617 | 50.876 | 84.373 | −30.326 | 1.00 | 0.00 | N |
| ATOM | 4865 | CA | ARG | A 617 | 50.933 | 82.938 | −30.038 | 1.00 | 0.00 | C |
| ATOM | 4866 | C | ARG | A 617 | 51.527 | 82.156 | −31.204 | 1.00 | 0.00 | C |
| ATOM | 4867 | O | ARG | A 617 | 51.039 | 82.267 | −32.328 | 1.00 | 0.00 | O |
| ATOM | 4868 | CB | ARG | A 617 | 49.529 | 82.368 | −29.786 | 1.00 | 0.00 | C |
| ATOM | 4869 | CG | ARG | A 617 | 48.870 | 82.713 | −28.464 | 1.00 | 0.00 | C |
| ATOM | 4870 | CD | ARG | A 617 | 47.479 | 82.073 | −28.383 | 1.00 | 0.00 | C |
| ATOM | 4871 | NE | ARG | A 617 | 47.529 | 80.609 | −28.395 | 1.00 | 0.00 | N |
| ATOM | 4872 | CZ | ARG | A 617 | 46.860 | 79.838 | −29.248 | 1.00 | 0.00 | C |
| ATOM | 4873 | NH1 | ARG | A 617 | 46.081 | 80.380 | −30.174 | 1.00 | 0.00 | N |
| ATOM | 4874 | NH2 | ARG | A 617 | 46.967 | 78.518 | −29.177 | 1.00 | 0.00 | N |
| ATOM | 4875 | N | ILE | A 618 | 52.577 | 81.375 | −30.954 | 1.00 | 0.00 | N |
| ATOM | 4876 | CA | ILE | A 618 | 53.130 | 80.547 | −32.019 | 1.00 | 0.00 | C |
| ATOM | 4877 | C | ILE | A 618 | 52.770 | 79.112 | −31.659 | 1.00 | 0.00 | C |
| ATOM | 4878 | O | ILE | A 618 | 52.944 | 78.683 | −30.519 | 1.00 | 0.00 | O |
| ATOM | 4879 | CB | ILE | A 618 | 54.664 | 80.704 | −32.188 | 1.00 | 0.00 | C |
| ATOM | 4880 | CG1 | ILE | A 618 | 55.113 | 79.892 | −33.409 | 1.00 | 0.00 | C |
| ATOM | 4881 | CG2 | ILE | A 618 | 55.399 | 80.266 | −30.932 | 1.00 | 0.00 | C |
| ATOM | 4882 | CD1 | ILE | A 618 | 56.471 | 80.272 | −33.954 | 1.00 | 0.00 | C |
| ATOM | 4883 | N | ILE | A 619 | 52.242 | 78.385 | −32.636 | 1.00 | 0.00 | N |
| ATOM | 4884 | CA | ILE | A 619 | 51.798 | 77.015 | −32.435 | 1.00 | 0.00 | C |
| ATOM | 4885 | C | ILE | A 619 | 52.481 | 76.048 | −33.395 | 1.00 | 0.00 | C |
| ATOM | 4886 | O | ILE | A 619 | 52.639 | 76.348 | −34.577 | 1.00 | 0.00 | O |
| ATOM | 4887 | CB | ILE | A 619 | 50.283 | 76.918 | −32.673 | 1.00 | 0.00 | C |
| ATOM | 4888 | CG1 | ILE | A 619 | 49.564 | 77.999 | −31.860 | 1.00 | 0.00 | C |
| ATOM | 4889 | CG2 | ILE | A 619 | 49.785 | 75.526 | −32.320 | 1.00 | 0.00 | C |
| ATOM | 4890 | CD1 | ILE | A 619 | 48.230 | 78.428 | −32.458 | 1.00 | 0.00 | C |
| ATOM | 4891 | N | PHE | A 620 | 52.876 | 74.884 | −32.890 | 1.00 | 0.00 | N |
| ATOM | 4892 | CA | PHE | A 620 | 53.509 | 73.883 | −33.735 | 1.00 | 0.00 | C |
| ATOM | 4893 | C | PHE | A 620 | 53.364 | 72.491 | −33.137 | 1.00 | 0.00 | C |
| ATOM | 4894 | O | PHE | A 620 | 53.086 | 72.341 | −31.951 | 1.00 | 0.00 | O |
| ATOM | 4895 | CB | PHE | A 620 | 54.987 | 74.227 | −33.971 | 1.00 | 0.00 | C |
| ATOM | 4896 | CG | PHE | A 620 | 55.854 | 74.113 | −32.749 | 1.00 | 0.00 | C |
| ATOM | 4897 | CD1 | PHE | A 620 | 56.454 | 72.899 | −32.417 | 1.00 | 0.00 | C |
| ATOM | 4898 | CD2 | PHE | A 620 | 56.097 | 75.222 | −31.946 | 1.00 | 0.00 | C |
| ATOM | 4899 | CE1 | PHE | A 620 | 57.289 | 72.791 | −31.303 | 1.00 | 0.00 | C |
| ATOM | 4900 | CE2 | PHE | A 620 | 56.931 | 75.127 | −30.829 | 1.00 | 0.00 | C |
| ATOM | 4901 | CZ | PHE | A 620 | 57.528 | 73.906 | −30.510 | 1.00 | 0.00 | C |
| ATOM | 4902 | N | LYS | A 621 | 53.539 | 71.475 | −33.971 | 1.00 | 0.00 | N |
| ATOM | 4903 | CA | LYS | A 621 | 53.406 | 70.098 | −33.526 | 1.00 | 0.00 | C |
| ATOM | 4904 | C | LYS | A 621 | 54.693 | 69.547 | −32.935 | 1.00 | 0.00 | C |
| ATOM | 4905 | O | LYS | A 621 | 55.716 | 69.474 | −33.613 | 1.00 | 0.00 | O |
| ATOM | 4906 | CB | LYS | A 621 | 52.946 | 69.220 | −34.698 | 1.00 | 0.00 | C |
| ATOM | 4907 | CG | LYS | A 621 | 52.735 | 67.753 | −34.337 | 1.00 | 0.00 | C |
| ATOM | 4908 | CD | LYS | A 621 | 51.891 | 67.034 | −35.387 | 1.00 | 0.00 | C |
| ATOM | 4909 | CE | LYS | A 621 | 52.554 | 67.047 | −36.750 | 1.00 | 0.00 | C |
| ATOM | 4910 | NZ | LYS | A 621 | 51.698 | 66.394 | −37.786 | 1.00 | 0.00 | N |
| ATOM | 4911 | N | ALA | A 622 | 54.645 | 69.171 | −31.660 | 1.00 | 0.00 | N |
| ATOM | 4912 | CA | ALA | A 622 | 55.815 | 68.601 | −31.007 | 1.00 | 0.00 | C |
| ATOM | 4913 | C | ALA | A 622 | 55.676 | 67.080 | −31.018 | 1.00 | 0.00 | C |
| ATOM | 4914 | O | ALA | A 622 | 54.578 | 66.550 | −30.856 | 1.00 | 0.00 | O |
| ATOM | 4915 | CB | ALA | A 622 | 55.928 | 69.113 | −29.564 | 1.00 | 0.00 | C |
| ATOM | 4916 | N | ARG | A 623 | 56.789 | 66.389 | −31.237 | 1.00 | 0.00 | N |
| ATOM | 4917 | CA | ARG | A 623 | 56.809 | 64.931 | −31.257 | 1.00 | 0.00 | C |
| ATOM | 4918 | C | ARG | A 623 | 57.802 | 64.529 | −30.174 | 1.00 | 0.00 | C |
| ATOM | 4919 | O | ARG | A 623 | 58.988 | 64.854 | −30.250 | 1.00 | 0.00 | O |
| ATOM | 4920 | CB | ARG | A 623 | 57.247 | 64.423 | −32.636 | 1.00 | 0.00 | C |
| ATOM | 4921 | CG | ARG | A 623 | 57.277 | 62.907 | −32.753 | 1.00 | 0.00 | C |
| ATOM | 4922 | CD | ARG | A 623 | 57.293 | 62.460 | −34.214 | 1.00 | 0.00 | C |
| ATOM | 4923 | NE | ARG | A 623 | 57.529 | 61.025 | −34.334 | 1.00 | 0.00 | N |
| ATOM | 4924 | CZ | ARG | A 623 | 58.725 | 60.456 | −34.234 | 1.00 | 0.00 | C |
| ATOM | 4925 | NH1 | ARG | A 623 | 59.802 | 61.201 | −34.019 | 1.00 | 0.00 | N |
| ATOM | 4926 | NH2 | ARG | A 623 | 58.845 | 59.139 | −34.333 | 1.00 | 0.00 | N |
| ATOM | 4927 | N | VAL | A 624 | 57.309 | 63.822 | −29.164 | 1.00 | 0.00 | N |
| ATOM | 4928 | CA | VAL | A 624 | 58.133 | 63.445 | −28.022 | 1.00 | 0.00 | C |
| ATOM | 4929 | C | VAL | A 624 | 58.204 | 61.941 | −27.775 | 1.00 | 0.00 | C |
| ATOM | 4930 | O | VAL | A 624 | 57.208 | 61.240 | −27.908 | 1.00 | 0.00 | O |
| ATOM | 4931 | CB | VAL | A 624 | 57.582 | 64.122 | −26.749 | 1.00 | 0.00 | C |
| ATOM | 4932 | CG1 | VAL | A 624 | 58.634 | 64.116 | −25.643 | 1.00 | 0.00 | C |
| ATOM | 4933 | CG2 | VAL | A 624 | 57.131 | 65.543 | −27.077 | 1.00 | 0.00 | C |
| ATOM | 4934 | N | PRO | A 625 | 59.388 | 61.435 | −27.393 | 1.00 | 0.00 | N |
| ATOM | 4935 | CA | PRO | A 625 | 59.579 | 60.007 | −27.123 | 1.00 | 0.00 | C |
| ATOM | 4936 | C | PRO | A 625 | 58.762 | 59.551 | −25.914 | 1.00 | 0.00 | C |
| ATOM | 4937 | O | PRO | A 625 | 58.336 | 60.367 | −25.097 | 1.00 | 0.00 | O |
| ATOM | 4938 | CB | PRO | A 625 | 61.079 | 59.903 | −26.843 | 1.00 | 0.00 | C |
| ATOM | 4939 | CG | PRO | A 625 | 61.664 | 61.071 | −27.591 | 1.00 | 0.00 | C |
| ATOM | 4940 | CD | PRO | A 625 | 60.667 | 62.158 | −27.296 | 1.00 | 0.00 | C |
| ATOM | 4941 | N | PRO | A 626 | 58.542 | 58.233 | −25.782 | 1.00 | 0.00 | N |
| ATOM | 4942 | CA | PRO | A 626 | 57.777 | 57.728 | −24.639 | 1.00 | 0.00 | C |
| ATOM | 4943 | C | PRO | A 626 | 58.528 | 58.166 | −23.378 | 1.00 | 0.00 | C |
| ATOM | 4944 | O | PRO | A 626 | 59.748 | 58.027 | −23.317 | 1.00 | 0.00 | O |
| ATOM | 4945 | CB | PRO | A 626 | 57.840 | 56.210 | −24.814 | 1.00 | 0.00 | C |
| ATOM | 4946 | CG | PRO | A 626 | 58.102 | 56.018 | −26.293 | 1.00 | 0.00 | C |
| ATOM | 4947 | CD | PRO | A 626 | 59.072 | 57.127 | −26.599 | 1.00 | 0.00 | C |
| ATOM | 4948 | N | MET | A 627 | 57.813 | 58.693 | −22.387 | 1.00 | 0.00 | N |
| ATOM | 4949 | CA | MET | A 627 | 58.444 | 59.126 | −21.140 | 1.00 | 0.00 | C |
| ATOM | 4950 | C | MET | A 627 | 59.744 | 59.879 | −21.427 | 1.00 | 0.00 | C |
| ATOM | 4951 | O | MET | A 627 | 60.747 | 59.703 | −20.726 | 1.00 | 0.00 | O |
| ATOM | 4952 | CB | MET | A 627 | 58.727 | 57.897 | −20.268 | 1.00 | 0.00 | C |
| ATOM | 4953 | CG | MET | A 627 | 57.460 | 57.170 | −19.822 | 1.00 | 0.00 | C |
| ATOM | 4954 | SD | MET | A 627 | 57.760 | 55.517 | −19.145 | 1.00 | 0.00 | S |
| ATOM | 4955 | CE | MET | A 627 | 58.542 | 55.917 | −17.584 | 1.00 | 0.00 | C |
| ATOM | 4956 | N | GLY | A 628 | 59.716 | 60.734 | −22.449 | 1.00 | 0.00 | N |
| ATOM | 4957 | CA | GLY | A 628 | 60.916 | 61.459 | −22.828 | 1.00 | 0.00 | C |
| ATOM | 4958 | C | GLY | A 628 | 60.818 | 62.955 | −23.043 | 1.00 | 0.00 | C |
| ATOM | 4959 | O | GLY | A 628 | 59.837 | 63.596 | −22.660 | 1.00 | 0.00 | O |
| ATOM | 4960 | N | LEU | A 629 | 61.855 | 63.506 | −23.673 | 1.00 | 0.00 | N |
| ATOM | 4961 | CA | LEU | A 629 | 61.943 | 64.938 | −23.939 | 1.00 | 0.00 | C |
| ATOM | 4962 | C | LEU | A 629 | 62.367 | 65.222 | −25.376 | 1.00 | 0.00 | C |
| ATOM | 4963 | O | LEU | A 629 | 63.060 | 64.416 | −25.999 | 1.00 | 0.00 | O |
| ATOM | 4964 | CB | LEU | A 629 | 62.967 | 65.569 | −22.996 | 1.00 | 0.00 | C |
| ATOM | 4965 | CG | LEU | A 629 | 62.727 | 65.359 | −21.500 | 1.00 | 0.00 | C |
| ATOM | 4966 | CD1 | LEU | A 629 | 63.989 | 65.688 | −20.720 | 1.00 | 0.00 | C |
| ATOM | 4967 | CD2 | LEU | A 629 | 61.562 | 66.231 | −21.051 | 1.00 | 0.00 | C |
| ATOM | 4968 | N | ALA | A 630 | 61.948 | 66.373 | −25.893 | 1.00 | 0.00 | N |
| ATOM | 4969 | CA | ALA | A 630 | 62.306 | 66.792 | −27.245 | 1.00 | 0.00 | C |
| ATOM | 4970 | C | ALA | A 630 | 62.533 | 68.297 | −27.216 | 1.00 | 0.00 | C |
| ATOM | 4971 | O | ALA | A 630 | 61.705 | 69.054 | −26.694 | 1.00 | 0.00 | O |
| ATOM | 4972 | CB | ALA | A 630 | 61.200 | 66.438 | −28.232 | 1.00 | 0.00 | C |
| ATOM | 4973 | N | THR | A 631 | 63.657 | 68.723 | −27.782 | 1.00 | 0.00 | N |
| ATOM | 4974 | CA | THR | A 631 | 64.033 | 70.135 | −27.802 | 1.00 | 0.00 | C |
| ATOM | 4975 | C | THR | A 631 | 63.782 | 70.816 | −29.148 | 1.00 | 0.00 | C |
| ATOM | 4976 | O | THR | A 631 | 64.047 | 70.238 | −30.199 | 1.00 | 0.00 | O |
| ATOM | 4977 | CB | THR | A 631 | 65.532 | 70.285 | −27.462 | 1.00 | 0.00 | C |
| ATOM | 4978 | OG1 | THR | A 631 | 65.813 | 69.582 | −26.244 | 1.00 | 0.00 | O |
| ATOM | 4979 | CG2 | THR | A 631 | 65.912 | 71.753 | −27.297 | 1.00 | 0.00 | C |
| ATOM | 4980 | N | TYR | A 632 | 63.267 | 72.044 | −29.103 | 1.00 | 0.00 | N |
| ATOM | 4981 | CA | TYR | A 632 | 63.025 | 72.825 | −30.311 | 1.00 | 0.00 | C |
| ATOM | 4982 | C | TYR | A 632 | 63.620 | 74.215 | −30.124 | 1.00 | 0.00 | C |
| ATOM | 4983 | O | TYR | A 632 | 63.964 | 74.606 | −29.008 | 1.00 | 0.00 | O |
| ATOM | 4984 | CB | TYR | A 632 | 61.527 | 72.942 | −30.615 | 1.00 | 0.00 | C |
| ATOM | 4985 | CG | TYR | A 632 | 60.869 | 71.626 | −30.965 | 1.00 | 0.00 | C |
| ATOM | 4986 | CD1 | TYR | A 632 | 60.488 | 70.728 | −29.968 | 1.00 | 0.00 | C |
| ATOM | 4987 | CD2 | TYR | A 632 | 60.656 | 71.264 | −32.296 | 1.00 | 0.00 | C |
| ATOM | 4988 | CE1 | TYR | A 632 | 59.913 | 69.501 | −30.286 | 1.00 | 0.00 | C |
| ATOM | 4989 | CE2 | TYR | A 632 | 60.081 | 70.036 | −32.627 | 1.00 | 0.00 | C |
| ATOM | 4990 | CZ | TYR | A 632 | 59.715 | 69.161 | −31.615 | 1.00 | 0.00 | C |
| ATOM | 4991 | OH | TYR | A 632 | 59.164 | 67.939 | −31.929 | 1.00 | 0.00 | O |
| ATOM | 4992 | N | VAL | A 633 | 63.742 | 74.957 | −31.219 | 1.00 | 0.00 | N |
| ATOM | 4993 | CA | VAL | A 633 | 64.299 | 76.302 | −31.171 | 1.00 | 0.00 | C |
| ATOM | 4994 | C | VAL | A 633 | 63.381 | 77.286 | −31.893 | 1.00 | 0.00 | C |
| ATOM | 4995 | O | VAL | A 633 | 62.901 | 77.005 | −32.992 | 1.00 | 0.00 | O |
| ATOM | 4996 | CB | VAL | A 633 | 65.698 | 76.350 | −31.838 | 1.00 | 0.00 | C |
| ATOM | 4997 | CG1 | VAL | A 633 | 66.253 | 77.779 | −31.808 | 1.00 | 0.00 | C |
| ATOM | 4998 | CG2 | VAL | A 633 | 66.644 | 75.390 | −31.126 | 1.00 | 0.00 | C |
| ATOM | 4999 | N | LEU | A 634 | 63.122 | 78.424 | −31.259 | 1.00 | 0.00 | N |
| ATOM | 5000 | CA | LEU | A 634 | 62.285 | 79.460 | −31.860 | 1.00 | 0.00 | C |
| ATOM | 5001 | C | LEU | A 634 | 63.227 | 80.586 | −32.265 | 1.00 | 0.00 | C |
| ATOM | 5002 | O | LEU | A 634 | 63.986 | 81.092 | −31.440 | 1.00 | 0.00 | O |
| ATOM | 5003 | CB | LEU | A 634 | 61.258 | 79.987 | −30.857 | 1.00 | 0.00 | C |
| ATOM | 5004 | CG | LEU | A 634 | 60.331 | 78.963 | −30.195 | 1.00 | 0.00 | C |
| ATOM | 5005 | CD1 | LEU | A 634 | 59.227 | 79.711 | −29.454 | 1.00 | 0.00 | C |
| ATOM | 5006 | CD2 | LEU | A 634 | 59.732 | 78.032 | −31.232 | 1.00 | 0.00 | C |
| ATOM | 5007 | N | THR | A 635 | 63.177 | 80.970 | −33.537 | 1.00 | 0.00 | N |
| ATOM | 5008 | CA | THR | A 635 | 64.052 | 82.013 | −34.053 | 1.00 | 0.00 | C |
| ATOM | 5009 | C | THR | A 635 | 63.256 | 83.135 | −34.708 | 1.00 | 0.00 | C |
| ATOM | 5010 | O | THR | A 635 | 62.308 | 82.884 | −35.453 | 1.00 | 0.00 | O |
| ATOM | 5011 | CB | THR | A 635 | 65.021 | 81.427 | −35.093 | 1.00 | 0.00 | C |
| ATOM | 5012 | OG1 | THR | A 635 | 65.704 | 80.303 | −34.522 | 1.00 | 0.00 | O |
| ATOM | 5013 | CG2 | THR | A 635 | 66.038 | 82.473 | −35.531 | 1.00 | 0.00 | C |
| ATOM | 5014 | N | ILE | A 636 | 63.651 | 84.374 | −34.435 | 1.00 | 0.00 | N |
| ATOM | 5015 | CA | ILE | A 636 | 62.964 | 85.525 | −35.006 | 1.00 | 0.00 | C |
| ATOM | 5016 | C | ILE | A 636 | 63.622 | 85.921 | −36.323 | 1.00 | 0.00 | C |
| ATOM | 5017 | O | ILE | A 636 | 64.797 | 85.637 | −36.549 | 1.00 | 0.00 | O |
| ATOM | 5018 | CB | ILE | A 636 | 63.003 | 86.734 | −34.035 | 1.00 | 0.00 | C |
| ATOM | 5019 | CG1 | ILE | A 636 | 62.149 | 87.884 | −34.580 | 1.00 | 0.00 | C |
| ATOM | 5020 | CG2 | ILE | A 636 | 64.437 | 87.198 | −33.844 | 1.00 | 0.00 | C |
| ATOM | 5021 | CD1 | ILE | A 636 | 62.018 | 89.059 | −33.625 | 1.00 | 0.00 | C |
| ATOM | 5022 | N | SER | A 637 | 62.851 | 86.554 | −37.200 | 1.00 | 0.00 | N |
| ATOM | 5023 | CA | SER | A 637 | 63.367 | 87.015 | −38.484 | 1.00 | 0.00 | C |
| ATON | 5024 | C | SER | A 637 | 62.768 | 88.392 | −38.757 | 1.00 | 0.00 | C |
| ATOM | 5025 | O | SER | A 637 | 61.829 | 88.805 | −38.081 | 1.00 | 0.00 | O |
| ATOM | 5026 | CB | SER | A 637 | 63.001 | 86.037 | −39.607 | 1.00 | 0.00 | C |
| ATOM | 5027 | OG | SER | A 637 | 61.600 | 85.878 | −39.721 | 1.00 | 0.00 | O |
| ATOM | 5028 | N | ASP | A 638 | 63.313 | 89.104 | −39.737 | 1.00 | 0.00 | N |
| ATOM | 5029 | CA | ASP | A 638 | 62.814 | 90.437 | −40.054 | 1.00 | 0.00 | C |
| ATOM | 5030 | C | ASP | A 638 | 61.445 | 90.401 | −40.724 | 1.00 | 0.00 | C |
| ATOM | 5031 | O | ASP | A 638 | 60.641 | 91.319 | −40.557 | 1.00 | 0.00 | O |
| ATOM | 5032 | CB | ASP | A 638 | 63.814 | 91.184 | −40.946 | 1.00 | 0.00 | C |
| ATOM | 5033 | CG | ASP | A 638 | 64.097 | 90.456 | −42.247 | 1.00 | 0.00 | C |
| ATOM | 5034 | OD1 | ASP | A 638 | 63.141 | 90.188 | −43.006 | 1.00 | 0.00 | O |
| ATOM | 5035 | OD2 | ASP | A 638 | 65.281 | 90.154 | −42.515 | 1.00 | 0.00 | O |
| ATOM | 5036 | N | SER | A 639 | 61.177 | 89.333 | −41.468 | 1.00 | 0.00 | N |
| ATOM | 5037 | CA | SER | A 639 | 59.906 | 89.197 | −42.170 | 1.00 | 0.00 | C |
| ATOM | 5038 | C | SER | A 639 | 59.308 | 87.806 | −42.009 | 1.00 | 0.00 | C |
| ATOM | 5039 | O | SER | A 639 | 59.907 | 86.930 | −41.390 | 1.00 | 0.00 | O |
| ATOM | 5040 | CB | SER | A 639 | 60.101 | 89.501 | −43.657 | 1.00 | 0.00 | C |
| ATOM | 5041 | OG | SER | A 639 | 61.062 | 88.629 | −44.224 | 1.00 | 0.00 | O |
| ATOM | 5042 | N | LYS | A 640 | 58.124 | 87.609 | −42.581 | 1.00 | 0.00 | N |
| ATOM | 5043 | CA | LYS | A 640 | 57.435 | 86.329 | −42.496 | 1.00 | 0.00 | C |
| ATOM | 5044 | C | LYS | A 640 | 58.293 | 85.145 | −42.929 | 1.00 | 0.00 | C |
| ATOM | 5045 | O | LYS | A 640 | 58.708 | 85.052 | −44.083 | 1.00 | 0.00 | O |
| ATOM | 5046 | CB | LYS | A 640 | 56.150 | 86.359 | −43.329 | 1.00 | 0.00 | C |
| ATOM | 5047 | CG | LYS | A 640 | 55.125 | 87.366 | −42.832 | 1.00 | 0.00 | C |
| ATOM | 5048 | CD | LYS | A 640 | 53.752 | 87.146 | −43.454 | 1.00 | 0.00 | C |
| ATOM | 5049 | CE | LYS | A 640 | 53.760 | 87.376 | −44.953 | 1.00 | 0.00 | C |
| ATOM | 5050 | NZ | LYS | A 640 | 52.393 | 87.210 | −45.517 | 1.00 | 0.00 | N |
| ATOM | 5051 | N | PRO | A 641 | 58.573 | 84.222 | −41.995 | 1.00 | 0.00 | N |
| ATOM | 5052 | CA | PRO | A 641 | 59.382 | 83.038 | −42.291 | 1.00 | 0.00 | C |
| ATOM | 5053 | C | PRO | A 641 | 58.627 | 82.057 | −43.186 | 1.00 | 0.00 | C |
| ATOM | 5054 | O | PRO | A 641 | 57.397 | 81.998 | −43.172 | 1.00 | 0.00 | O |
| ATOM | 5055 | CB | PRO | A 641 | 59.677 | 82.470 | −40.905 | 1.00 | 0.00 | C |
| ATOM | 5056 | CG | PRO | A 641 | 58.464 | 82.841 | −40.133 | 1.00 | 0.00 | C |
| ATOM | 5057 | CD | PRO | A 641 | 58.206 | 84.263 | −40.569 | 1.00 | 0.00 | C |
| ATOM | 5058 | N | GLU | A 642 | 59.379 | 81.284 | −43.959 | 1.00 | 0.00 | N |
| ATOM | 5059 | CA | GLU | A 642 | 58.810 | 80.318 | −44.889 | 1.00 | 0.00 | C |
| ATOM | 5060 | C | GLU | A 642 | 57.877 | 79.260 | −44.298 | 1.00 | 0.00 | C |
| ATOM | 5061 | O | GLU | A 642 | 56.848 | 78.940 | −44.892 | 1.00 | 0.00 | O |
| ATOM | 5062 | CB | GLU | A 642 | 59.943 | 79.614 | −45.649 | 1.00 | 0.00 | C |
| ATOM | 5063 | CG | GLU | A 642 | 59.481 | 78.483 | −46.557 | 1.00 | 0.00 | C |
| ATOM | 5064 | CD | GLU | A 642 | 60.631 | 77.788 | −47.269 | 1.00 | 0.00 | C |
| ATOM | 5065 | OE1 | GLU | A 642 | 60.372 | 76.790 | −47.977 | 1.00 | 0.00 | O |
| ATOM | 5066 | OE2 | GLU | A 642 | 61.790 | 78.236 | −47.124 | 1.00 | 0.00 | O |
| ATOM | 5067 | N | HIS | A 643 | 58.220 | 78.722 | −43.135 | 1.00 | 0.00 | N |
| ATOM | 5068 | CA | HIS | A 643 | 57.409 | 77.663 | −42.538 | 1.00 | 0.00 | C |
| ATOM | 5069 | C | HIS | A 643 | 56.417 | 78.079 | −41.460 | 1.00 | 0.00 | C |
| ATOM | 5070 | O | HIS | A 643 | 55.942 | 77.240 | −40.692 | 1.00 | 0.00 | O |
| ATOM | 5071 | CB | HIS | A 643 | 58.333 | 76.575 | −41.996 | 1.00 | 0.00 | C |
| ATOM | 5072 | CG | HIS | A 643 | 59.246 | 76.001 | −43.034 | 1.00 | 0.00 | C |
| ATOM | 5073 | ND1 | HIS | A 643 | 58.823 | 75.079 | −43.967 | 1.00 | 0.00 | N |
| ATOM | 5074 | CD2 | HIS | A 643 | 60.545 | 76.254 | −43.317 | 1.00 | 0.00 | C |
| ATOM | 5075 | CE1 | HIS | A 643 | 59.823 | 74.789 | −44.781 | 1.00 | 0.00 | C |
| ATOM | 5076 | NE2 | HIS | A 643 | 60.879 | 75.489 | −44.408 | 1.00 | 0.00 | N |
| ATOM | 5077 | N | THR | A 644 | 56.102 | 79.367 | −41.405 | 1.00 | 0.00 | N |
| ATOM | 5078 | CA | THR | A 644 | 55.149 | 79.868 | −40.426 | 1.00 | 0.00 | C |
| ATOM | 5079 | C | THR | A 644 | 53.990 | 80.546 | −41.153 | 1.00 | 0.00 | C |
| ATOM | 5080 | O | THR | A 644 | 54.205 | 81.423 | −41.993 | 1.00 | 0.00 | O |
| ATOM | 5081 | CB | THR | A 644 | 55.810 | 80.880 | −39.468 | 1.00 | 0.00 | C |
| ATOM | 5082 | OG1 | THR | A 644 | 56.890 | 80.239 | −38.773 | 1.00 | 0.00 | O |
| ATOM | 5083 | CG2 | THR | A 644 | 54.795 | 81.402 | −38.446 | 1.00 | 0.00 | C |
| ATOM | 5084 | N | SER | A 645 | 52.768 | 80.118 | −40.848 | 1.00 | 0.00 | N |
| ATOM | 5085 | CA | SER | A 645 | 51.580 | 80.699 | −41.462 | 1.00 | 0.00 | C |
| ATOM | 5086 | C | SER | A 645 | 50.860 | 81.552 | −40.424 | 1.00 | 0.00 | C |
| ATOM | 5087 | O | SER | A 645 | 51.122 | 81.440 | −39.221 | 1.00 | 0.00 | O |
| ATOM | 5088 | CB | SER | A 645 | 50.642 | 79.601 | −41.983 | 1.00 | 0.00 | C |
| ATOM | 5089 | OG | SER | A 645 | 50.111 | 78.815 | −40.927 | 1.00 | 0.00 | O |
| ATOM | 5090 | N | TYR | A 646 | 49.953 | 82.405 | −40.887 | 1.00 | 0.00 | N |
| ATOM | 5091 | CA | TYR | A 646 | 49.213 | 83.274 | −39.983 | 1.00 | 0.00 | C |
| ATOM | 5092 | C | TYR | A 646 | 47.712 | 83.080 | −40.098 | 1.00 | 0.00 | C |
| ATOM | 5093 | O | TYR | A 646 | 47.155 | 83.078 | −41.194 | 1.00 | 0.00 | O |
| ATOM | 5094 | CB | TYR | A 646 | 49.593 | 84.733 | −40.243 | 1.00 | 0.00 | C |
| ATOM | 5095 | CG | TYR | A 646 | 51.072 | 84.959 | −40.046 | 1.00 | 0.00 | C |
| ATOM | 5096 | CD1 | TYR | A 646 | 51.983 | 84.665 | −41.061 | 1.00 | 0.00 | C |
| ATOM | 5097 | CD2 | TYR | A 646 | 51.571 | 85.368 | −38.812 | 1.00 | 0.00 | C |
| ATOM | 5098 | CE1 | TYR | A 646 | 53.355 | 84.767 | −40.846 | 1.00 | 0.00 | C |
| ATOM | 5099 | CE2 | TYR | A 646 | 52.938 | 85.472 | −38.586 | 1.00 | 0.00 | C |
| ATOM | 5100 | CZ | TYR | A 646 | 53.823 | 85.169 | −39.604 | 1.00 | 0.00 | C |
| ATOM | 5101 | OH | TYR | A 646 | 55.177 | 85.251 | −39.374 | 1.00 | 0.00 | O |
| ATOM | 5102 | N | ALA | A 647 | 47.066 | 82.908 | −38.951 | 1.00 | 0.00 | N |
| ATOM | 5103 | CA | ALA | A 647 | 45.630 | 82.694 | −38.904 | 1.00 | 0.00 | C |
| ATOM | 5104 | C | ALA | A 647 | 44.842 | 83.937 | −39.288 | 1.00 | 0.00 | C |
| ATOM | 5105 | O | ALA | A 647 | 45.283 | 85.064 | −39.072 | 1.00 | 0.00 | O |
| ATOM | 5106 | CB | ALA | A 647 | 45.222 | 82.247 | −37.505 | 1.00 | 0.00 | C |
| ATOM | 5107 | N | SER | A 648 | 43.674 | 83.722 | −39.873 | 1.00 | 0.00 | N |
| ATOM | 5108 | CA | SER | A 648 | 42.808 | 84.831 | −40.229 | 1.00 | 0.00 | C |
| ATOM | 5109 | C | SER | A 648 | 41.916 | 84.985 | −39.005 | 1.00 | 0.00 | C |
| ATOM | 5110 | O | SER | A 648 | 41.749 | 84.034 | −38.235 | 1.00 | 0.00 | O |
| ATOM | 5111 | CB | SER | A 648 | 41.963 | 84.486 | −41.458 | 1.00 | 0.00 | C |
| ATOM | 5112 | OG | SER | A 648 | 41.118 | 83.382 | −41.195 | 1.00 | 0.00 | O |
| ATOM | 5113 | N | ASN | A 649 | 41.351 | 86.171 | −38.812 | 1.00 | 0.00 | N |
| ATOM | 5114 | CA | ASN | A 649 | 40.481 | 86.411 | −37.668 | 1.00 | 0.00 | C |
| ATOM | 5115 | C | ASN | A 649 | 39.236 | 87.155 | −38.125 | 1.00 | 0.00 | C |
| ATOM | 5116 | O | ASN | A 649 | 39.321 | 88.127 | −38.878 | 1.00 | 0.00 | O |
| ATOM | 5117 | CB | ASN | A 649 | 41.216 | 87.231 | −36.602 | 1.00 | 0.00 | C |
| ATOM | 5118 | CG | ASN | A 649 | 42.398 | 86.488 | −36.000 | 1.00 | 0.00 | C |
| ATOM | 5119 | OD1 | ASN | A 649 | 42.253 | 85.738 | −35.031 | 1.00 | 0.00 | O |
| ATOM | 5120 | ND2 | ASN | A 649 | 43.575 | 86.687 | −36.581 | 1.00 | 0.00 | N |
| ATOM | 5121 | N | LEU | A 650 | 38.083 | 86.689 | −37.660 | 1.00 | 0.00 | N |
| ATOM | 5122 | CA | LEU | A 650 | 36.803 | 87.291 | −38.013 | 1.00 | 0.00 | C |
| ATOM | 5123 | C | LEU | A 650 | 35.982 | 87.568 | −36.763 | 1.00 | 0.00 | C |
| ATOM | 5124 | O | LEU | A 650 | 35.622 | 86.647 | −36.022 | 1.00 | 0.00 | O |
| ATOM | 5125 | CB | LEU | A 650 | 36.027 | 86.356 | −38.941 | 1.00 | 0.00 | C |
| ATOM | 5126 | CG | LEU | A 650 | 34.612 | 86.784 | −39.335 | 1.00 | 0.00 | C |
| ATOM | 5127 | CD1 | LEU | A 650 | 34.666 | 88.039 | −40.200 | 1.00 | 0.00 | C |
| ATOM | 5128 | CD2 | LEU | A 650 | 33.935 | 85.646 | −40.082 | 1.00 | 0.00 | C |
| ATOM | 5129 | N | LEU | A 651 | 35.683 | 88.843 | −36.533 | 1.00 | 0.00 | N |
| ATOM | 5130 | CA | LEU | A 651 | 34.905 | 89.254 | −35.373 | 1.00 | 0.00 | C |
| ATOM | 5131 | C | LEU | A 651 | 33.467 | 89.502 | −35.817 | 1.00 | 0.00 | C |
| ATOM | 5132 | O | LEU | A 651 | 33.185 | 90.456 | −36.544 | 1.00 | 0.00 | O |
| ATOM | 5133 | CB | LEU | A 651 | 35.508 | 90.530 | −34.772 | 1.00 | 0.00 | C |
| ATOM | 5134 | CG | LEU | A 651 | 35.144 | 90.909 | −33.330 | 1.00 | 0.00 | C |
| ATOM | 5135 | CD1 | LEU | A 651 | 33.701 | 91.351 | −33.240 | 1.00 | 0.00 | C |
| ATOM | 5136 | CD2 | LEU | A 651 | 35.404 | 89.721 | −32.421 | 1.00 | 0.00 | C |
| ATOM | 5137 | N | LEU | A 652 | 32.558 | 88.640 | −35.376 | 1.00 | 0.00 | N |
| ATOM | 5138 | CA | LEU | A 652 | 31.159 | 88.760 | −35.750 | 1.00 | 0.00 | C |
| ATOM | 5139 | C | LEU | A 652 | 30.315 | 89.452 | −34.695 | 1.00 | 0.00 | C |
| ATOM | 5140 | O | LEU | A 652 | 30.211 | 88.992 | −33.553 | 1.00 | 0.00 | O |
| ATOM | 5141 | CB | LEU | A 652 | 30.577 | 87.378 | −36.048 | 1.00 | 0.00 | C |
| ATOM | 5142 | CG | LEU | A 652 | 31.305 | 86.607 | −37.150 | 1.00 | 0.00 | C |
| ATOM | 5143 | CD1 | LEU | A 652 | 30.711 | 85.211 | −37.279 | 1.00 | 0.00 | C |
| ATOM | 5144 | CD2 | LEU | A 652 | 31.202 | 87.372 | −38.472 | 1.00 | 0.00 | C |
| ATOM | 5145 | N | ARG | A 653 | 29.718 | 90.571 | −35.088 | 1.00 | 0.00 | N |
| ATOM | 5146 | CA | ARG | A 653 | 28.859 | 91.332 | −34.198 | 1.00 | 0.00 | C |
| ATOM | 5147 | C | ARG | A 653 | 28.429 | 92.626 | −34.861 | 1.00 | 0.00 | C |
| ATOM | 5148 | O | ARG | A 653 | 29.052 | 93.084 | −35.817 | 1.00 | 0.00 | O |
| ATOM | 5149 | CB | ARG | A 653 | 29.581 | 91.662 | −32.896 | 1.00 | 0.00 | C |
| ATOM | 5150 | CG | ARG | A 653 | 30.650 | 92.725 | −33.015 | 1.00 | 0.00 | C |
| ATOM | 5151 | CD | ARG | A 653 | 30.507 | 93.692 | −31.860 | 1.00 | 0.00 | C |
| ATOM | 5152 | NE | ARG | A 653 | 31.785 | 94.047 | −31.264 | 1.00 | 0.00 | N |
| ATOM | 5153 | CZ | ARG | A 653 | 31.909 | 94.744 | −30.140 | 1.00 | 0.00 | C |
| ATOM | 5154 | NH1 | ARG | A 653 | 30.827 | 95.157 | −29.493 | 1.00 | 0.00 | N |
| ATOM | 5155 | NH2 | ARG | A 653 | 33.113 | 95.022 | −29.662 | 1.00 | 0.00 | N |
| ATOM | 5156 | N | LYS | A 654 | 27.357 | 93.210 | −34.343 | 1.00 | 0.00 | N |
| ATOM | 5157 | CA | LYS | A 654 | 26.856 | 94.466 | −34.869 | 1.00 | 0.00 | C |
| ATOM | 5158 | C | LYS | A 654 | 27.641 | 95.583 | −34.191 | 1.00 | 0.00 | C |
| ATOM | 5159 | O | LYS | A 654 | 28.143 | 95.409 | −33.079 | 1.00 | 0.00 | O |
| ATOM | 5160 | CB | LYS | A 654 | 25.364 | 94.609 | −34.562 | 1.00 | 0.00 | C |
| ATOM | 5161 | CG | LYS | A 654 | 24.502 | 93.497 | −35.154 | 1.00 | 0.00 | C |
| ATOM | 5162 | CD | LYS | A 654 | 23.395 | 94.057 | −36.038 | 1.00 | 0.00 | C |
| ATOM | 5163 | CE | LYS | A 654 | 23.968 | 94.808 | −37.232 | 1.00 | 0.00 | C |
| ATOM | 5164 | NZ | LYS | A 654 | 22.898 | 95.390 | −38.094 | 1.00 | 0.00 | N |
| ATOM | 5165 | N | ASN | A 655 | 27.757 | 96.719 | −34.867 | 1.00 | 0.00 | N |
| ATOM | 5166 | CA | ASN | A 655 | 28.474 | 97.865 | −34.322 | 1.00 | 0.00 | C |
| ATOM | 5167 | C | ASN | A 655 | 29.898 | 97.502 | −33.912 | 1.00 | 0.00 | C |
| ATOM | 5168 | O | ASN | A 655 | 30.283 | 97.665 | −32.754 | 1.00 | 0.00 | O |
| ATOM | 5169 | CB | ASN | A 655 | 27.720 | 98.421 | −33.115 | 1.00 | 0.00 | C |
| ATOM | 5170 | CG | ASN | A 655 | 26.229 | 98.523 | −33.363 | 1.00 | 0.00 | C |
| ATOM | 5171 | OD1 | ASN | A 655 | 25.790 | 99.109 | −34.357 | 1.00 | 0.00 | O |
| ATOM | 5172 | ND2 | ASN | A 655 | 25.438 | 97.949 | −32.460 | 1.00 | 0.00 | N |
| ATOM | 5173 | N | PRO | A 656 | 30.701 | 97.002 | −34.860 | 1.00 | 0.00 | N |
| ATOM | 5174 | CA | PRO | A 656 | 32.080 | 96.631 | −34.543 | 1.00 | 0.00 | C |
| ATOM | 5175 | C | PRO | A 656 | 33.011 | 97.837 | −34.570 | 1.00 | 0.00 | C |
| ATOM | 5176 | O | PRO | A 656 | 32.703 | 98.858 | −35.185 | 1.00 | 0.00 | O |
| ATOM | 5177 | CB | PRO | A 656 | 32.419 | 95.638 | −35.643 | 1.00 | 0.00 | C |
| ATOM | 5178 | CG | PRO | A 656 | 31.722 | 96.248 | −36.824 | 1.00 | 0.00 | C |
| ATOM | 5179 | CD | PRO | A 656 | 30.366 | 96.626 | −36.247 | 1.00 | 0.00 | C |
| ATOM | 5180 | N | THR | A 657 | 34.144 | 97.709 | −33.890 | 1.00 | 0.00 | N |
| ATOM | 5181 | CA | THR | A 657 | 35.152 | 98.758 | −33.862 | 1.00 | 0.00 | C |
| ATOM | 5182 | C | THR | A 657 | 36.462 | 98.098 | −34.278 | 1.00 | 0.00 | C |
| ATOM | 5183 | O | THR | A 657 | 36.618 | 96.882 | −34.146 | 1.00 | 0.00 | O |
| ATOM | 5184 | CB | THR | A 657 | 35.300 | 99.380 | −32.453 | 1.00 | 0.00 | C |
| ATOM | 5185 | OG1 | THR | A 657 | 35.484 | 98.342 | −31.483 | 1.00 | 0.00 | O |
| ATOM | 5186 | CG2 | THR | A 657 | 34.064 | 100.200 | −32.100 | 1.00 | 0.00 | C |
| ATOM | 5187 | N | SER | A 658 | 37.392 | 98.895 | −34.788 | 1.00 | 0.00 | N |
| ATOM | 5188 | CA | SER | A 658 | 38.676 | 98.379 | −35.240 | 1.00 | 0.00 | C |
| ATOM | 5189 | C | SER | A 658 | 39.399 | 97.561 | −34.172 | 1.00 | 0.00 | C |
| ATOM | 5190 | O | SER | A 658 | 39.192 | 97.753 | −32.973 | 1.00 | 0.00 | O |
| ATOM | 5191 | CB | SER | A 658 | 39.570 | 99.533 | −35.697 | 1.00 | 0.00 | C |
| ATOM | 5192 | OG | SER | A 658 | 39.842 | 100.410 | −34.620 | 1.00 | 0.00 | O |
| ATOM | 5193 | N | LEU | A 659 | 40.244 | 96.641 | −34.631 | 1.00 | 0.00 | N |
| ATOM | 5194 | CA | LEU | A 659 | 41.024 | 95.777 | −33.753 | 1.00 | 0.00 | C |
| ATOM | 5195 | C | LEU | A 659 | 42.437 | 95.645 | −34.309 | 1.00 | 0.00 | C |
| ATOM | 5196 | O | LEU | A 659 | 42.755 | 94.674 | −34.999 | 1.00 | 0.00 | O |
| ATOM | 5197 | CB | LEU | A 659 | 40.383 | 94.387 | −33.660 | 1.00 | 0.00 | C |
| ATOM | 5198 | CG | LEU | A 659 | 39.074 | 94.254 | −32.876 | 1.00 | 0.00 | C |
| ATOM | 5199 | CD1 | LEU | A 659 | 38.458 | 92.877 | −33.115 | 1.00 | 0.00 | C |
| ATOM | 5200 | CD2 | LEU | A 659 | 39.347 | 94.470 | −31.401 | 1.00 | 0.00 | C |
| ATOM | 5201 | N | PRO | A 660 | 43.302 | 96.635 | −34.035 | 1.00 | 0.00 | N |
| ATOM | 5202 | CA | PRO | A 660 | 44.684 | 96.595 | −34.524 | 1.00 | 0.00 | C |
| ATOM | 5203 | C | PRO | A 660 | 45.469 | 95.503 | −33.795 | 1.00 | 0.00 | C |
| ATOM | 5204 | O | PRO | A 660 | 45.168 | 95.181 | −32.648 | 1.00 | 0.00 | O |
| ATOM | 5205 | CB | PRO | A 660 | 45.196 | 97.999 | −34.211 | 1.00 | 0.00 | C |
| ATOM | 5206 | CG | PRO | A 660 | 44.461 | 98.341 | −32.954 | 1.00 | 0.00 | C |
| ATOM | 5207 | CD | PRO | A 660 | 43.052 | 97.863 | −33.260 | 1.00 | 0.00 | C |
| ATOM | 5208 | N | LEU | A 661 | 46.472 | 94.938 | −34.457 | 1.00 | 0.00 | N |
| ATOM | 5209 | CA | LEU | A 661 | 47.259 | 93.869 | −33.848 | 1.00 | 0.00 | C |
| ATOM | 5210 | C | LEU | A 661 | 48.769 | 94.042 | −34.012 | 1.00 | 0.00 | C |
| ATOM | 5211 | O | LEU | A 661 | 49.494 | 93.070 | −34.227 | 1.00 | 0.00 | O |
| ATOM | 5212 | CB | LEU | A 661 | 46.826 | 92.522 | −34.435 | 1.00 | 0.00 | C |
| ATOM | 5213 | CG | LEU | A 661 | 45.370 | 92.120 | −34.193 | 1.00 | 0.00 | C |
| ATOM | 5214 | CD1 | LEU | A 661 | 45.041 | 90.862 | −34.987 | 1.00 | 0.00 | C |
| ATOM | 5215 | CD2 | LEU | A 661 | 45.144 | 91.895 | −32.704 | 1.00 | 0.00 | C |
| ATOM | 5216 | N | GLY | A 662 | 49.240 | 95.279 | −33.907 | 1.00 | 0.00 | N |
| ATOM | 5217 | CA | GLY | A 662 | 50.662 | 95.534 | −34.046 | 1.00 | 0.00 | C |
| ATOM | 5218 | C | GLY | A 662 | 51.253 | 95.010 | −35.341 | 1.00 | 0.00 | C |
| ATOM | 5219 | O | GLY | A 662 | 50.788 | 95.351 | −36.427 | 1.00 | 0.00 | O |
| ATOM | 5220 | N | GLN | A 663 | 52.272 | 94.163 | −35.225 | 1.00 | 0.00 | N |
| ATOM | 5221 | CA | GLN | A 663 | 52.953 | 93.599 | −36.388 | 1.00 | 0.00 | C |
| ATOM | 5222 | C | GLN | A 663 | 52.231 | 92.438 | −37.065 | 1.00 | 0.00 | C |
| ATOM | 5223 | O | GLN | A 663 | 52.638 | 92.003 | −38.141 | 1.00 | 0.00 | O |
| ATOM | 5224 | CB | GLN | A 663 | 54.350 | 93.123 | −35.991 | 1.00 | 0.00 | C |
| ATOM | 5225 | CG | GLN | A 663 | 55.169 | 94.131 | −35.214 | 1.00 | 0.00 | C |
| ATOM | 5226 | CD | GLN | A 663 | 56.480 | 93.541 | −34.738 | 1.00 | 0.00 | C |
| ATOM | 5227 | OE1 | GLN | A 663 | 57.337 | 93.174 | −35.546 | 1.00 | 0.00 | O |
| ATOM | 5228 | NE2 | GLN | A 663 | 56.640 | 93.435 | −33.423 | 1.00 | 0.00 | N |
| ATOM | 5229 | N | TYR | A 664 | 51.173 | 91.929 | −36.444 | 1.00 | 0.00 | N |
| ATOM | 5230 | CA | TYR | A 664 | 50.444 | 90.800 | −37.016 | 1.00 | 0.00 | C |
| ATOM | 5231 | C | TYR | A 664 | 50.151 | 91.025 | −38.503 | 1.00 | 0.00 | C |
| ATOM | 5232 | O | TYR | A 664 | 49.513 | 92.006 | −38.883 | 1.00 | 0.00 | O |
| ATOM | 5233 | CB | TYR | A 664 | 49.143 | 90.562 | −36.247 | 1.00 | 0.00 | C |
| ATOM | 5234 | CG | TYR | A 664 | 48.556 | 89.186 | −36.471 | 1.00 | 0.00 | C |
| ATOM | 5235 | CD1 | TYR | A 664 | 49.146 | 88.053 | −35.906 | 1.00 | 0.00 | C |
| ATOM | 5236 | CD2 | TYR | A 664 | 47.414 | 89.014 | −37.253 | 1.00 | 0.00 | C |
| ATOM | 5237 | CE1 | TYR | A 664 | 48.608 | 86.781 | −36.114 | 1.00 | 0.00 | C |
| ATOM | 5238 | CE2 | TYR | A 664 | 46.872 | 87.753 | −37.469 | 1.00 | 0.00 | C |
| ATOM | 5239 | CZ | TYR | A 664 | 47.471 | 86.639 | −36.896 | 1.00 | 0.00 | C |
| ATOM | 5240 | OH | TYR | A 664 | 46.924 | 85.393 | −37.105 | 1.00 | 0.00 | O |
| ATOM | 5241 | N | PRO | A 665 | 50.618 | 90.102 | −39.361 | 1.00 | 0.00 | N |
| ATOM | 5242 | CA | PRO | A 665 | 50.488 | 90.081 | −40.824 | 1.00 | 0.00 | C |
| ATOM | 5243 | C | PRO | A 665 | 49.109 | 90.299 | −41.447 | 1.00 | 0.00 | C |
| ATOM | 5244 | O | PRO | A 665 | 48.993 | 91.004 | −42.449 | 1.00 | 0.00 | O |
| ATOM | 5245 | CB | PRO | A 665 | 51.056 | 88.713 | −41.201 | 1.00 | 0.00 | C |
| ATOM | 5246 | CG | PRO | A 665 | 52.066 | 88.471 | −40.145 | 1.00 | 0.00 | C |
| ATOM | 5247 | CD | PRO | A 665 | 51.331 | 88.897 | −38.900 | 1.00 | 0.00 | C |
| ATOM | 5248 | N | GLU | A 666 | 48.069 | 89.695 | −40.878 | 1.00 | 0.00 | N |
| ATOM | 5249 | CA | GLU | A 666 | 46.736 | 89.837 | −41.455 | 1.00 | 0.00 | C |
| ATOM | 5250 | C | GLU | A 666 | 45.768 | 90.685 | −40.646 | 1.00 | 0.00 | C |
| ATOM | 5251 | O | GLU | A 666 | 45.704 | 90.584 | −39.421 | 1.00 | 0.00 | O |
| ATOM | 5252 | CB | GLU | A 666 | 46.112 | 88.460 | −41.706 | 1.00 | 0.00 | C |
| ATOM | 5253 | CG | GLU | A 666 | 44.867 | 88.537 | −42.578 | 1.00 | 0.00 | C |
| ATOM | 5254 | CD | GLU | A 666 | 44.401 | 87.187 | −43.078 | 1.00 | 0.00 | C |
| ATOM | 5255 | OE1 | GLU | A 666 | 45.226 | 86.454 | −43.664 | 1.00 | 0.00 | O |
| ATOM | 5256 | OE2 | GLU | A 666 | 43.208 | 86.866 | −42.895 | 1.00 | 0.00 | O |
| ATOM | 5257 | N | ASP | A 667 | 45.007 | 91.515 | −41.356 | 1.00 | 0.00 | N |
| ATOM | 5258 | CA | ASP | A 667 | 44.028 | 92.403 | −40.736 | 1.00 | 0.00 | C |
| ATOM | 5259 | C | ASP | A 667 | 42.768 | 91.654 | −40.320 | 1.00 | 0.00 | C |
| ATOM | 5260 | O | ASP | A 667 | 42.281 | 90.786 | −41.042 | 1.00 | 0.00 | O |
| ATOM | 5261 | CB | ASP | A 667 | 43.633 | 93.518 | −41.710 | 1.00 | 0.00 | C |
| ATOM | 5262 | CG | ASP | A 667 | 44.824 | 94.305 | −42.208 | 1.00 | 0.00 | C |
| ATOM | 5263 | OD1 | ASP | A 667 | 45.590 | 94.819 | −41.366 | 1.00 | 0.00 | O |
| ATOM | 5264 | OD2 | ASP | A 667 | 44.991 | 94.411 | −43.441 | 1.00 | 0.00 | O |
| ATOM | 5265 | N | VAL | A 668 | 42.240 | 92.002 | −39.157 | 1.00 | 0.00 | N |
| ATOM | 5266 | CA | VAL | A 668 | 41.024 | 91.375 | −38.661 | 1.00 | 0.00 | C |
| ATOM | 5267 | C | VAL | A 668 | 39.862 | 91.771 | −39.564 | 1.00 | 0.00 | C |
| ATOM | 5268 | O | VAL | A 668 | 39.807 | 92.902 | −40.054 | 1.00 | 0.00 | O |
| ATOM | 5269 | CB | VAL | A 668 | 40.714 | 91.830 | −37.222 | 1.00 | 0.00 | C |
| ATOM | 5270 | CG1 | VAL | A 668 | 39.391 | 91.232 | −36.756 | 1.00 | 0.00 | C |
| ATOM | 5271 | CG2 | VAL | A 668 | 41.850 | 91.410 | −36.291 | 1.00 | 0.00 | C |
| ATOM | 5272 | N | LYS | A 669 | 38.947 | 90.833 | −39.789 | 1.00 | 0.00 | N |
| ATOM | 5273 | CA | LYS | A 669 | 37.769 | 91.067 | −40.621 | 1.00 | 0.00 | C |
| ATOM | 5274 | C | LYS | A 669 | 36.545 | 91.132 | −39.712 | 1.00 | 0.00 | C |
| ATOM | 5275 | O | LYS | A 669 | 36.541 | 90.542 | −38.629 | 1.00 | 0.00 | O |
| ATOM | 5276 | CB | LYS | A 669 | 37.615 | 89.931 | −41.636 | 1.00 | 0.00 | C |
| ATOM | 5277 | CG | LYS | A 669 | 38.809 | 89.796 | −42.574 | 1.00 | 0.00 | C |
| ATOM | 5278 | CD | LYS | A 669 | 38.917 | 88.397 | −43.173 | 1.00 | 0.00 | C |
| ATOM | 5279 | CE | LYS | A 669 | 40.205 | 88.245 | −43.982 | 1.00 | 0.00 | C |
| ATOM | 5280 | NZ | LYS | A 669 | 40.454 | 86.840 | −44.416 | 1.00 | 0.00 | N |
| ATOM | 5281 | N | PHE | A 670 | 35.511 | 91.851 | −40.146 | 1.00 | 0.00 | N |
| ATOM | 5282 | CA | PHE | A 670 | 34.299 | 91.985 | −39.348 | 1.00 | 0.00 | C |
| ATOM | 5283 | C | PHE | A 670 | 33.047 | 91.602 | −40.129 | 1.00 | 0.00 | C |
| ATOM | 5284 | O | PHE | A 670 | 33.088 | 91.433 | −41.347 | 1.00 | 0.00 | O |
| ATOM | 5285 | CB | PHE | A 670 | 34.156 | 93.422 | −38.831 | 1.00 | 0.00 | C |
| ATOM | 5286 | CG | PHE | A 670 | 35.342 | 93.906 | −38.053 | 1.00 | 0.00 | C |
| ATOM | 5287 | CD1 | PHE | A 670 | 36.470 | 94.389 | −38.709 | 1.00 | 0.00 | C |
| ATOM | 5288 | CD2 | PHE | A 670 | 35.350 | 93.845 | −36.663 | 1.00 | 0.00 | C |
| ATOM | 5289 | CE1 | PHE | A 670 | 37.589 | 94.801 | −37.991 | 1.00 | 0.00 | C |
| ATOM | 5290 | CE2 | PHE | A 670 | 36.464 | 94.254 | −35.938 | 1.00 | 0.00 | C |
| ATOM | 5291 | CZ | PHE | A 670 | 37.586 | 94.733 | −36.604 | 1.00 | 0.00 | C |
| ATOM | 5292 | N | GLY | A 671 | 31.935 | 91.463 | −39.417 | 1.00 | 0.00 | N |
| ATOM | 5293 | CA | GLY | A 671 | 30.684 | 91.109 | −40.064 | 1.00 | 0.00 | C |
| ATOM | 5294 | C | GLY | A 671 | 29.565 | 90.909 | −39.065 | 1.00 | 0.00 | C |
| ATOM | 5295 | O | GLY | A 671 | 29.808 | 90.815 | −37.861 | 1.00 | 0.00 | O |
| ATOM | 5296 | N | ASP | A 672 | 28.331 | 90.854 | −39.554 | 1.00 | 0.00 | N |
| ATOM | 5297 | CA | ASP | A 672 | 27.197 | 90.638 | −38.671 | 1.00 | 0.00 | C |
| ATOM | 5298 | C | ASP | A 672 | 27.188 | 89.169 | −38.265 | 1.00 | 0.00 | C |
| ATOM | 5299 | O | ASP | A 672 | 27.696 | 88.315 | −38.990 | 1.00 | 0.00 | O |
| ATOM | 5300 | CB | ASP | A 672 | 25.873 | 90.954 | −39.373 | 1.00 | 0.00 | C |
| ATOM | 5301 | CG | ASP | A 672 | 25.705 | 92.427 | −39.684 | 1.00 | 0.00 | C |
| ATOM | 5302 | OD1 | ASP | A 672 | 26.368 | 93.262 | −39.036 | 1.00 | 0.00 | O |
| ATOM | 5303 | OD2 | ASP | A 672 | 24.885 | 92.749 | −40.570 | 1.00 | 0.00 | O |
| ATOM | 5304 | N | PRO | A 673 | 26.615 | 88.860 | −37.095 | 1.00 | 0.00 | N |
| ATOM | 5305 | CA | PRO | A 673 | 26.553 | 87.474 | −36.625 | 1.00 | 0.00 | C |
| ATOM | 5306 | C | PRO | A 673 | 25.942 | 86.587 | −37.711 | 1.00 | 0.00 | C |
| ATOM | 5307 | O | PRO | A 673 | 25.003 | 86.990 | −38.398 | 1.00 | 0.00 | O |
| ATOM | 5308 | CB | PRO | A 673 | 25.657 | 87.573 | −35.396 | 1.00 | 0.00 | C |
| ATOM | 5309 | CG | PRO | A 673 | 25.978 | 88.931 | −34.862 | 1.00 | 0.00 | C |
| ATOM | 5310 | CD | PRO | A 673 | 26.010 | 89.777 | −36.112 | 1.00 | 0.00 | C |
| ATOM | 5311 | N | ARG | A 674 | 26.488 | 85.389 | −37.875 | 1.00 | 0.00 | N |
| ATOM | 5312 | CA | ARG | A 674 | 25.984 | 84.457 | −38.876 | 1.00 | 0.00 | C |
| ATOM | 5313 | C | ARG | A 674 | 26.505 | 83.060 | −38.581 | 1.00 | 0.00 | C |
| ATOM | 5314 | O | ARG | A 674 | 27.494 | 82.901 | −37.867 | 1.00 | 0.00 | O |
| ATOM | 5315 | CB | ARG | A 674 | 26.428 | 84.876 | −40.280 | 1.00 | 0.00 | C |
| ATOM | 5316 | CG | ARG | A 674 | 27.925 | 84.753 | −40.542 | 1.00 | 0.00 | C |
| ATOM | 5317 | CD | ARG | A 674 | 28.206 | 84.818 | −42.036 | 1.00 | 0.00 | C |
| ATOM | 5318 | NE | ARG | A 674 | 29.585 | 84.475 | −42.371 | 1.00 | 0.00 | N |
| ATOM | 5319 | CZ | ARG | A 674 | 30.601 | 85.331 | −42.359 | 1.00 | 0.00 | C |
| ATOM | 5320 | NH1 | ARG | A 674 | 30.406 | 86.601 | −42.027 | 1.00 | 0.00 | N |
| ATOM | 5321 | NH2 | ARG | A 674 | 31.816 | 84.915 | −42.691 | 1.00 | 0.00 | N |
| ATOM | 5322 | N | GLU | A 675 | 25.841 | 82.047 | −39.125 | 1.00 | 0.00 | N |
| ATOM | 5323 | CA | GLU | A 675 | 26.286 | 80.682 | −38.903 | 1.00 | 0.00 | C |
| ATOM | 5324 | C | GLU | A 675 | 27.589 | 80.477 | −39.654 | 1.00 | 0.00 | C |
| ATOM | 5325 | O | GLU | A 675 | 27.811 | 81.080 | −40.708 | 1.00 | 0.00 | O |
| ATOM | 5326 | CB | GLU | A 675 | 25.238 | 79.679 | −39.388 | 1.00 | 0.00 | C |
| ATOM | 5327 | CG | GLU | A 675 | 23.878 | 79.856 | −38.740 | 1.00 | 0.00 | C |
| ATOM | 5328 | CD | GLU | A 675 | 23.065 | 78.578 | −38.738 | 1.00 | 0.00 | C |
| ATOM | 5329 | OE1 | GLU | A 675 | 23.073 | 77.863 | −39.763 | 1.00 | 0.00 | O |
| ATOM | 5330 | OE2 | GLU | A 675 | 22.412 | 78.296 | −37.709 | 1.00 | 0.00 | O |
| ATOM | 5331 | N | ILE | A 676 | 28.462 | 79.642 | −39.103 | 1.00 | 0.00 | N |
| ATOM | 5332 | CA | ILE | A 676 | 29.739 | 79.376 | −39.743 | 1.00 | 0.00 | C |
| ATOM | 5333 | C | ILE | A 676 | 30.089 | 77.902 | −39.660 | 1.00 | 0.00 | C |
| ATOM | 5334 | O | ILE | A 676 | 29.577 | 77.173 | −38.809 | 1.00 | 0.00 | O |
| ATOM | 5335 | CB | ILE | A 676 | 30.886 | 80.171 | −39.087 | 1.00 | 0.00 | C |
| ATOM | 5336 | CG1 | ILE | A 676 | 31.114 | 79.672 | −37.662 | 1.00 | 0.00 | C |
| ATOM | 5337 | CG2 | ILE | A 676 | 30.557 | 81.657 | −39.076 | 1.00 | 0.00 | C |
| ATOM | 5338 | CD1 | ILE | A 676 | 32.429 | 80.119 | −37.071 | 1.00 | 0.00 | C |
| ATOM | 5339 | N | SER | A 677 | 30.970 | 77.480 | −40.555 | 1.00 | 0.00 | N |
| ATOM | 5340 | CA | SER | A 677 | 31.427 | 76.103 | −40.610 | 1.00 | 0.00 | C |
| ATOM | 5341 | C | SER | A 677 | 32.948 | 76.133 | −40.674 | 1.00 | 0.00 | C |
| ATOM | 5342 | O | SER | A 677 | 33.528 | 77.013 | −41.308 | 1.00 | 0.00 | O |
| ATOM | 5343 | CB | SER | A 677 | 30.862 | 75.413 | −41.853 | 1.00 | 0.00 | C |
| ATOM | 5344 | OG | SER | A 677 | 31.334 | 74.083 | −41.948 | 1.00 | 0.00 | O |
| ATOM | 5345 | N | LEU | A 678 | 33.593 | 75.179 | −40.012 | 1.00 | 0.00 | N |
| ATOM | 5346 | CA | LEU | A 678 | 35.049 | 75.115 | −40.003 | 1.00 | 0.00 | C |
| ATOM | 5347 | C | LEU | A 678 | 35.549 | 73.683 | −40.098 | 1.00 | 0.00 | C |
| ATOM | 5348 | O | LEU | A 678 | 34.884 | 72.748 | −39.642 | 1.00 | 0.00 | O |
| ATOM | 5349 | CB | LEU | A 678 | 35.609 | 75.740 | −38.721 | 1.00 | 0.00 | C |
| ATOM | 5350 | CG | LEU | A 678 | 35.617 | 77.262 | −38.558 | 1.00 | 0.00 | C |
| ATOM | 5351 | CD1 | LEU | A 678 | 36.028 | 77.618 | −37.136 | 1.00 | 0.00 | C |
| ATOM | 5352 | CD2 | LEU | A 678 | 36.583 | 77.880 | −39.551 | 1.00 | 0.00 | C |
| ATOM | 5353 | N | ARG | A 679 | 36.728 | 73.527 | −40.690 | 1.00 | 0.00 | N |
| ATOM | 5354 | CA | ARG | A 679 | 37.353 | 72.222 | −40.828 | 1.00 | 0.00 | C |
| ATOM | 5355 | C | ARG | A 679 | 38.872 | 72.350 | −40.839 | 1.00 | 0.00 | C |
| ATOM | 5356 | O | ARG | A 679 | 39.442 | 73.052 | −41.678 | 1.00 | 0.00 | O |
| ATOM | 5357 | CB | ARG | A 679 | 36.904 | 71.527 | −42.114 | 1.00 | 0.00 | C |
| ATOM | 5358 | CG | ARG | A 679 | 37.394 | 70.091 | −42.189 | 1.00 | 0.00 | C |
| ATOM | 5359 | CD | ARG | A 679 | 37.126 | 69.442 | −43.533 | 1.00 | 0.00 | C |
| ATOM | 5360 | NE | ARG | A 679 | 37.546 | 68.044 | −43.514 | 1.00 | 0.00 | N |
| ATOM | 5361 | CZ | ARG | A 679 | 37.573 | 67.252 | −44.580 | 1.00 | 0.00 | C |
| ATOM | 5362 | NH1 | ARG | A 679 | 37.203 | 67.719 | −45.767 | 1.00 | 0.00 | N |
| ATOM | 5363 | NH2 | ARG | A 679 | 37.972 | 65.993 | −44.458 | 1.00 | 0.00 | N |
| ATOM | 5364 | N | VAL | A 680 | 39.525 | 71.676 | −39.898 | 1.00 | 0.00 | N |
| ATOM | 5365 | CA | VAL | A 680 | 40.978 | 71.697 | −39.822 | 1.00 | 0.00 | C |
| ATOM | 5366 | C | VAL | A 680 | 41.509 | 70.345 | −40.291 | 1.00 | 0.00 | C |
| ATOM | 5367 | O | VAL | A 680 | 41.008 | 69.298 | −39.876 | 1.00 | 0.00 | O |
| ATOM | 5368 | CB | VAL | A 680 | 41.459 | 71.964 | −38.384 | 1.00 | 0.00 | C |
| ATOM | 5369 | CG1 | VAL | A 680 | 42.976 | 71.884 | −38.318 | 1.00 | 0.00 | C |
| ATOM | 5370 | CG2 | VAL | A 680 | 40.980 | 73.341 | −37.928 | 1.00 | 0.00 | C |
| ATOM | 5371 | N | GLY | A 681 | 42.515 | 70.375 | −41.160 | 1.00 | 0.00 | N |
| ATOM | 5372 | CA | GLY | A 681 | 43.086 | 69.144 | −41.677 | 1.00 | 0.00 | C |
| ATOM | 5373 | C | GLY | A 681 | 42.016 | 68.264 | −42.298 | 1.00 | 0.00 | C |
| ATOM | 5374 | O | GLY | A 681 | 41.088 | 68.762 | −42.937 | 1.00 | 0.00 | O |
| ATOM | 5375 | N | ASN | A 682 | 42.142 | 66.954 | −42.112 | 1.00 | 0.00 | N |
| ATOM | 5376 | CA | ASN | A 682 | 41.165 | 66.013 | −42.649 | 1.00 | 0.00 | C |
| ATOM | 5377 | C | ASN | A 682 | 40.206 | 65.593 | −41.546 | 1.00 | 0.00 | C |
| ATOM | 5378 | O | ASN | A 682 | 39.487 | 64.603 | −41.677 | 1.00 | 0.00 | O |
| ATOM | 5379 | CB | ASN | A 682 | 41.863 | 64.776 | −43.216 | 1.00 | 0.00 | C |
| ATOM | 5380 | CG | ASN | A 682 | 42.643 | 65.072 | −44.480 | 1.00 | 0.00 | C |
| ATOM | 5381 | OD1 | ASN | A 682 | 43.187 | 64.166 | −45.108 | 1.00 | 0.00 | O |
| ATOM | 5382 | ND2 | ASN | A 682 | 42.702 | 66.343 | −44.861 | 1.00 | 0.00 | N |
| ATOM | 5383 | N | GLY | A 683 | 40.205 | 66.356 | −40.457 | 1.00 | 0.00 | N |
| ATOM | 5384 | CA | GLY | A 683 | 39.338 | 66.054 | −39.334 | 1.00 | 0.00 | C |
| ATOM | 5385 | C | GLY | A 683 | 37.875 | 66.320 | −39.620 | 1.00 | 0.00 | C |
| ATOM | 5386 | O | GLY | A 683 | 37.484 | 66.446 | −40.780 | 1.00 | 0.00 | O |
| ATOM | 5387 | N | PRO | A 684 | 37.035 | 66.410 | −38.577 | 1.00 | 0.00 | N |
| ATOM | 5388 | CA | PRO | A 684 | 35.605 | 66.664 | −38.763 | 1.00 | 0.00 | C |
| ATOM | 5389 | C | PRO | A 684 | 35.305 | 68.106 | −39.162 | 1.00 | 0.00 | C |
| ATOM | 5390 | O | PRO | A 684 | 36.141 | 68.994 | −39.005 | 1.00 | 0.00 | O |
| ATOM | 5391 | CB | PRO | A 684 | 35.018 | 66.315 | −37.400 | 1.00 | 0.00 | C |
| ATOM | 5392 | CG | PRO | A 684 | 36.104 | 66.759 | −36.464 | 1.00 | 0.00 | C |
| ATOM | 5393 | CD | PRO | A 684 | 37.358 | 66.243 | −37.147 | 1.00 | 0.00 | C |
| ATOM | 5394 | N | THR | A 685 | 34.109 | 68.320 | −39.695 | 1.00 | 0.00 | N |
| ATOM | 5395 | CA | THR | A 685 | 33.668 | 69.650 | −40.090 | 1.00 | 0.00 | C |
| ATOM | 5396 | C | THR | A 685 | 32.606 | 70.042 | −39.075 | 1.00 | 0.00 | C |
| ATOM | 5397 | O | THR | A 685 | 31.611 | 69.337 | −38.908 | 1.00 | 0.00 | O |
| ATOM | 5398 | CB | THR | A 685 | 33.050 | 69.647 | −41.504 | 1.00 | 0.00 | C |
| ATOM | 5399 | OG1 | THR | A 685 | 34.052 | 69.280 | −42.458 | 1.00 | 0.00 | O |
| ATOM | 5400 | CG2 | THR | A 685 | 32.500 | 71.028 | −41.853 | 1.00 | 0.00 | C |
| ATOM | 5401 | N | LEU | A 686 | 32.824 | 71.160 | −38.390 | 1.00 | 0.00 | N |
| ATOM | 5402 | CA | LEU | A 686 | 31.886 | 71.618 | −37.376 | 1.00 | 0.00 | C |
| ATOM | 5403 | C | LEU | A 686 | 31.119 | 72.858 | −37.813 | 1.00 | 0.00 | C |
| ATOM | 5404 | O | LEU | A 686 | 31.684 | 73.772 | −38.414 | 1.00 | 0.00 | O |
| ATOM | 5405 | CB | LEU | A 686 | 32.622 | 71.920 | −36.066 | 1.00 | 0.00 | C |
| ATOM | 5406 | CG | LEU | A 686 | 33.544 | 70.858 | −35.460 | 1.00 | 0.00 | C |
| ATOM | 5407 | CD1 | LEU | A 686 | 33.962 | 71.315 | −34.063 | 1.00 | 0.00 | C |
| ATOM | 5408 | CD2 | LEU | A 686 | 32.847 | 69.510 | −35.390 | 1.00 | 0.00 | C |
| ATOM | 5409 | N | ALA | A 687 | 29.828 | 72.875 | −37.502 | 1.00 | 0.00 | N |
| ATOM | 5410 | CA | ALA | A 687 | 28.964 | 73.997 | −37.841 | 1.00 | 0.00 | C |
| ATOM | 5411 | C | ALA | A 687 | 28.488 | 74.652 | −36.553 | 1.00 | 0.00 | C |
| ATOM | 5412 | O | ALA | A 687 | 28.122 | 73.965 | −35.597 | 1.00 | 0.00 | O |
| ATOM | 5413 | CB | ALA | A 687 | 27.769 | 73.517 | −38.657 | 1.00 | 0.00 | C |
| ATOM | 5414 | N | PHE | A 688 | 28.491 | 75.980 | −36.536 | 1.00 | 0.00 | N |
| ATOM | 5415 | CA | PHE | A 688 | 28.074 | 76.739 | −35.367 | 1.00 | 0.00 | C |
| ATOM | 5416 | C | PHE | A 688 | 26.922 | 77.683 | −35.689 | 1.00 | 0.00 | C |
| ATOM | 5417 | O | PHE | A 688 | 26.763 | 78.122 | −36.829 | 1.00 | 0.00 | O |
| ATOM | 5418 | CB | PHE | A 688 | 29.242 | 77.572 | −34.828 | 1.00 | 0.00 | C |
| ATOM | 5419 | CG | PHE | A 688 | 30.478 | 76.775 | −34.529 | 1.00 | 0.00 | C |
| ATOM | 5420 | CD1 | PHE | A 688 | 31.288 | 76.305 | −35.558 | 1.00 | 0.00 | C |
| ATOM | 5421 | CD2 | PHE | A 688 | 30.837 | 76.501 | −33.213 | 1.00 | 0.00 | C |
| ATOM | 5422 | CE1 | PHE | A 688 | 32.441 | 75.576 | −35.282 | 1.00 | 0.00 | C |
| ATOM | 5423 | CE2 | PHE | A 688 | 31.987 | 75.772 | −32.925 | 1.00 | 0.00 | C |
| ATOM | 5424 | CZ | PHE | A 688 | 32.792 | 75.308 | −33.961 | 1.00 | 0.00 | C |
| ATOM | 5425 | N | SER | A 689 | 26.130 | 78.001 | −34.673 | 1.00 | 0.00 | N |
| ATOM | 5426 | CA | SER | A 689 | 25.007 | 78.917 | −34.834 | 1.00 | 0.00 | C |
| ATOM | 5427 | C | SER | A 689 | 25.576 | 80.335 | −34.813 | 1.00 | 0.00 | C |
| ATOM | 5428 | O | SER | A 689 | 26.767 | 80.527 | −34.558 | 1.00 | 0.00 | O |
| ATOM | 5429 | CB | SER | A 689 | 24.016 | 78.744 | −33.684 | 1.00 | 0.00 | C |
| ATOM | 5430 | OG | SER | A 689 | 24.568 | 79.221 | −32.469 | 1.00 | 0.00 | O |
| ATOM | 5431 | N | GLU | A 690 | 24.731 | 81.326 | −35.079 | 1.00 | 0.00 | N |
| ATOM | 5432 | CA | GLU | A 690 | 25.187 | 82.710 | −35.082 | 1.00 | 0.00 | C |
| ATOM | 5433 | C | GLU | A 690 | 25.540 | 83.190 | −33.674 | 1.00 | 0.00 | C |
| ATOM | 5434 | O | GLU | A 690 | 26.025 | 84.308 | −33.495 | 1.00 | 0.00 | O |
| ATOM | 5435 | CB | GLU | A 690 | 24.126 | 83.628 | −35.699 | 1.00 | 0.00 | C |
| ATOM | 5436 | CG | GLU | A 690 | 22.857 | 83.776 | −34.885 | 1.00 | 0.00 | C |
| ATOM | 5437 | CD | GLU | A 690 | 21.918 | 84.811 | −35.478 | 1.00 | 0.00 | C |
| ATOM | 5438 | OE1 | GLU | A 690 | 21.437 | 84.597 | −36.612 | 1.00 | 0.00 | O |
| ATOM | 5439 | OE2 | GLU | A 690 | 21.670 | 85.841 | −34.814 | 1.00 | 0.00 | O |
| ATOM | 5440 | N | GLN | A 691 | 25.287 | 82.347 | −32.677 | 1.00 | 0.00 | N |
| ATOM | 5441 | CA | GLN | A 691 | 25.619 | 82.678 | −31.294 | 1.00 | 0.00 | C |
| ATOM | 5442 | C | GLN | A 691 | 26.954 | 82.023 | −30.928 | 1.00 | 0.00 | C |
| ATOM | 5443 | O | GLN | A 691 | 27.361 | 82.025 | −29.767 | 1.00 | 0.00 | O |
| ATOM | 5444 | CB | GLN | A 691 | 24.525 | 82.188 | −30.345 | 1.00 | 0.00 | C |
| ATOM | 5445 | CG | GLN | A 691 | 23.212 | 82.931 | −30.489 | 1.00 | 0.00 | C |
| ATOM | 5446 | CD | GLN | A 691 | 22.019 | 82.012 | −30.371 | 1.00 | 0.00 | C |
| ATOM | 5447 | OE1 | GLN | A 691 | 21.820 | 81.356 | −29.346 | 1.00 | 0.00 | O |
| ATOM | 5448 | NE2 | GLN | A 691 | 21.215 | 81.954 | −31.429 | 1.00 | 0.00 | N |
| ATOM | 5449 | N | GLY | A 692 | 27.623 | 81.457 | −31.927 | 1.00 | 0.00 | N |
| ATOM | 5450 | CA | GLY | A 692 | 28.913 | 80.826 | −31.701 | 1.00 | 0.00 | C |
| ATOM | 5451 | C | GLY | A 692 | 28.879 | 79.488 | −30.984 | 1.00 | 0.00 | C |
| ATOM | 5452 | O | GLY | A 692 | 29.877 | 79.068 | −30.399 | 1.00 | 0.00 | O |
| ATOM | 5453 | N | LEU | A 693 | 27.738 | 78.811 | −31.028 | 1.00 | 0.00 | N |
| ATOM | 5454 | CA | LEU | A 693 | 27.599 | 77.515 | −30.371 | 1.00 | 0.00 | C |
| ATOM | 5455 | C | LEU | A 693 | 27.504 | 76.373 | −31.371 | 1.00 | 0.00 | C |
| ATOM | 5456 | O | LEU | A 693 | 26.909 | 76.513 | −32.443 | 1.00 | 0.00 | O |
| ATOM | 5457 | CB | LEU | A 693 | 26.363 | 77.519 | −29.470 | 1.00 | 0.00 | C |
| ATOM | 5458 | CG | LEU | A 693 | 26.458 | 78.426 | −28.240 | 1.00 | 0.00 | C |
| ATOM | 5459 | CD1 | LEU | A 693 | 25.062 | 78.762 | −27.736 | 1.00 | 0.00 | C |
| ATOM | 5460 | CD2 | LEU | A 693 | 27.278 | 77.743 | −27.162 | 1.00 | 0.00 | C |
| ATOM | 5461 | N | LEU | A 694 | 28.090 | 75.236 | −31.009 | 1.00 | 0.00 | N |
| ATOM | 5462 | CA | LEU | A 694 | 28.083 | 74.058 | −31.865 | 1.00 | 0.00 | C |
| ATOM | 5463 | C | LEU | A 694 | 26.657 | 73.654 | −32.222 | 1.00 | 0.00 | C |
| ATOM | 5464 | O | LEU | A 694 | 25.765 | 73.697 | −31.378 | 1.00 | 0.00 | O |
| ATOM | 5465 | CB | LEU | A 694 | 28.783 | 72.893 | −31.160 | 1.00 | 0.00 | C |
| ATOM | 5466 | CG | LEU | A 694 | 28.965 | 71.617 | −31.987 | 1.00 | 0.00 | C |
| ATOM | 5467 | CD1 | LEU | A 694 | 29.942 | 71.878 | −33.125 | 1.00 | 0.00 | C |
| ATOM | 5468 | CD2 | LEU | A 694 | 29.488 | 70.498 | −31.089 | 1.00 | 0.00 | C |
| ATOM | 5469 | N | LYS | A 695 | 26.457 | 73.265 | −33.478 | 1.00 | 0.00 | N |
| ATOM | 5470 | CA | LYS | A 695 | 25.146 | 72.847 | −33.960 | 1.00 | 0.00 | C |
| ATOM | 5471 | C | LYS | A 695 | 25.196 | 71.465 | −34.599 | 1.00 | 0.00 | C |
| ATOM | 5472 | O | LYS | A 695 | 24.229 | 70.710 | −34.524 | 1.00 | 0.00 | O |
| ATOM | 5473 | CB | LYS | A 695 | 24.611 | 73.867 | −34.971 | 1.00 | 0.00 | C |
| ATOM | 5474 | CG | LYS | A 695 | 23.343 | 73.447 | −35.694 | 1.00 | 0.00 | C |
| ATOM | 5475 | CD | LYS | A 695 | 22.666 | 74.640 | −36.374 | 1.00 | 0.00 | C |
| ATOM | 5476 | CE | LYS | A 695 | 22.146 | 75.636 | −35.340 | 1.00 | 0.00 | C |
| ATOM | 5477 | NZ | LYS | A 695 | 21.441 | 76.797 | −35.955 | 1.00 | 0.00 | N |
| ATOM | 5478 | N | SER | A 696 | 26.323 | 71.134 | −35.226 | 1.00 | 0.00 | N |
| ATOM | 5479 | CA | SER | A 696 | 26.471 | 69.836 | −35.876 | 1.00 | 0.00 | C |
| ATOM | 5480 | C | SER | A 696 | 27.928 | 69.449 | −36.092 | 1.00 | 0.00 | C |
| ATOM | 5481 | O | SER | A 696 | 28.814 | 70.306 | −36.134 | 1.00 | 0.00 | O |
| ATOM | 5482 | CB | SER | A 696 | 25.753 | 69.839 | −37.230 | 1.00 | 0.00 | C |
| ATOM | 5483 | OG | SER | A 696 | 26.466 | 70.610 | −38.183 | 1.00 | 0.00 | O |
| ATOM | 5484 | N | ILE | A 697 | 28.159 | 68.146 | −36.234 | 1.00 | 0.00 | N |
| ATOM | 5485 | CA | ILE | A 697 | 29.490 | 67.598 | −36.467 | 1.00 | 0.00 | C |
| ATOM | 5486 | C | ILE | A 697 | 29.441 | 66.634 | −37.653 | 1.00 | 0.00 | C |
| ATOM | 5487 | O | ILE | A 697 | 28.601 | 65.731 | −37.687 | 1.00 | 0.00 | O |
| ATOM | 5488 | CB | ILE | A 697 | 30.003 | 66.793 | −35.248 | 1.00 | 0.00 | C |
| ATOM | 5489 | CG1 | ILE | A 697 | 30.124 | 67.696 | −34.020 | 1.00 | 0.00 | C |
| ATOM | 5490 | CG2 | ILE | A 697 | 31.349 | 66.157 | −35.573 | 1.00 | 0.00 | C |
| ATOM | 5491 | CD1 | ILE | A 697 | 30.600 | 66.963 | −32.774 | 1.00 | 0.00 | C |
| ATOM | 5492 | N | GLN | A 698 | 30.330 | 66.831 | −38.620 | 1.00 | 0.00 | N |
| ATOM | 5493 | CA | GLN | A 698 | 30.404 | 65.949 | −39.779 | 1.00 | 0.00 | C |
| ATOM | 5494 | C | GLN | A 698 | 31.751 | 65.240 | −39.717 | 1.00 | 0.00 | C |
| ATOM | 5495 | O | GLN | A 698 | 32.796 | 65.856 | −39.939 | 1.00 | 0.00 | O |
| ATOM | 5496 | CB | GLN | A 698 | 30.302 | 66.735 | −41.090 | 1.00 | 0.00 | C |
| ATOM | 5497 | CG | GLN | A 698 | 29.993 | 65.836 | −42.286 | 1.00 | 0.00 | C |
| ATOM | 5498 | CD | GLN | A 698 | 30.220 | 66.514 | −43.624 | 1.00 | 0.00 | C |
| ATOM | 5499 | OE1 | GLN | A 698 | 29.673 | 66.092 | −44.645 | 1.00 | 0.00 | O |
| ATOM | 5500 | NE2 | GLN | A 698 | 31.038 | 67.557 | −43.629 | 1.00 | 0.00 | N |
| ATOM | 5501 | N | LEU | A 699 | 31.725 | 63.945 | −39.419 | 1.00 | 0.00 | N |
| ATOM | 5502 | CA | LEU | A 699 | 32.949 | 63.162 | −39.295 | 1.00 | 0.00 | C |
| ATOM | 5503 | C | LEU | A 699 | 33.788 | 63.092 | −40.564 | 1.00 | 0.00 | C |
| ATOM | 5504 | O | LEU | A 699 | 35.012 | 63.210 | −40.510 | 1.00 | 0.00 | O |
| ATOM | 5505 | CB | LEU | A 699 | 32.618 | 61.746 | −38.814 | 1.00 | 0.00 | C |
| ATOM | 5506 | CG | LEU | A 699 | 31.900 | 61.656 | −37.462 | 1.00 | 0.00 | C |
| ATOM | 5507 | CD1 | LEU | A 699 | 31.706 | 60.197 | −37.080 | 1.00 | 0.00 | C |
| ATOM | 5508 | CD2 | LEU | A 699 | 32.714 | 62.377 | −36.399 | 1.00 | 0.00 | C |
| ATOM | 5509 | N | THR | A 700 | 33.138 | 62.891 | −41.704 | 1.00 | 0.00 | N |
| ATOM | 5510 | CA | THR | A 700 | 33.853 | 62.810 | −42.973 | 1.00 | 0.00 | C |
| ATOM | 5511 | C | THR | A 700 | 33.128 | 63.618 | −44.040 | 1.00 | 0.00 | C |
| ATOM | 5512 | O | THR | A 700 | 31.962 | 63.969 | −43.875 | 1.00 | 0.00 | O |
| ATOM | 5513 | CB | THR | A 700 | 33.976 | 61.348 | −43.454 | 1.00 | 0.00 | C |
| ATOM | 5514 | OG1 | THR | A 700 | 32.669 | 60.792 | −43.647 | 1.00 | 0.00 | O |
| ATOM | 5515 | CG2 | THR | A 700 | 34.730 | 60.518 | −42.431 | 1.00 | 0.00 | C |
| ATOM | 5516 | N | GLN | A 701 | 33.824 | 63.915 | −45.132 | 1.00 | 0.00 | N |
| ATOM | 5517 | CA | GLN | A 701 | 33.237 | 64.689 | −46.221 | 1.00 | 0.00 | C |
| ATOM | 5518 | C | GLN | A 701 | 32.010 | 63.982 | −46.783 | 1.00 | 0.00 | C |
| ATOM | 5519 | O | GLN | A 701 | 31.122 | 64.617 | −47.350 | 1.00 | 0.00 | O |
| ATOM | 5520 | CB | GLN | A 701 | 34.263 | 64.897 | −47.336 | 1.00 | 0.00 | C |
| ATOM | 5521 | CG | GLN | A 701 | 35.585 | 65.458 | −46.852 | 1.00 | 0.00 | C |
| ATOM | 5522 | CD | GLN | A 701 | 36.471 | 65.922 | −47.990 | 1.00 | 0.00 | C |
| ATOM | 5523 | OE1 | GLN | A 701 | 36.103 | 66.821 | −48.749 | 1.00 | 0.00 | O |
| ATOM | 5524 | NE2 | GLN | A 701 | 37.648 | 65.313 | −48.114 | 1.00 | 0.00 | N |
| ATOM | 5525 | N | ASP | A 702 | 31.968 | 62.666 | −46.613 | 1.00 | 0.00 | N |
| ATOM | 5526 | CA | ASP | A 702 | 30.858 | 61.857 | −47.104 | 1.00 | 0.00 | C |
| ATOM | 5527 | C | ASP | A 702 | 29.703 | 61.853 | −46.107 | 1.00 | 0.00 | C |
| ATOM | 5528 | O | ASP | A 702 | 28.558 | 62.142 | −46.459 | 1.00 | 0.00 | O |
| ATOM | 5529 | CB | ASP | A 702 | 31.327 | 60.418 | −47.327 | 1.00 | 0.00 | C |
| ATOM | 5530 | CG | ASP | A 702 | 32.718 | 60.344 | −47.923 | 1.00 | 0.00 | C |
| ATOM | 5531 | OD1 | ASP | A 702 | 32.907 | 60.816 | −49.067 | 1.00 | 0.00 | O |
| ATOM | 5532 | OD2 | ASP | A 702 | 33.625 | 59.816 | −47.241 | 1.00 | 0.00 | O |
| ATOM | 5533 | N | SER | A 703 | 30.022 | 61.520 | −44.860 | 1.00 | 0.00 | N |
| ATOM | 5534 | CA | SER | A 703 | 29.037 | 61.449 | −43.788 | 1.00 | 0.00 | C |
| ATOM | 5535 | C | SER | A 703 | 28.156 | 62.691 | −43.688 | 1.00 | 0.00 | C |
| ATOM | 5536 | O | SER | A 703 | 28.481 | 63.747 | −44.229 | 1.00 | 0.00 | O |
| ATOM | 5537 | CB | SER | A 703 | 29.747 | 61.215 | −42.452 | 1.00 | 0.00 | C |
| ATOM | 5538 | OG | SER | A 703 | 30.677 | 62.249 | −42.182 | 1.00 | 0.00 | O |
| ATOM | 5539 | N | PRO | A 704 | 27.016 | 62.571 | −42.989 | 1.00 | 0.00 | N |
| ATOM | 5540 | CA | PRO | A 704 | 26.075 | 63.676 | −42.807 | 1.00 | 0.00 | C |
| ATOM | 5541 | C | PRO | A 704 | 26.439 | 64.570 | −41.625 | 1.00 | 0.00 | C |
| ATOM | 5542 | O | PRO | A 704 | 27.338 | 64.253 | −40.845 | 1.00 | 0.00 | O |
| ATOM | 5543 | CB | PRO | A 704 | 24.757 | 62.955 | −42.583 | 1.00 | 0.00 | C |
| ATOM | 5544 | CG | PRO | A 704 | 25.187 | 61.782 | −41.767 | 1.00 | 0.00 | C |
| ATOM | 5545 | CD | PRO | A 704 | 26.429 | 61.308 | −42.500 | 1.00 | 0.00 | C |
| ATOM | 5546 | N | HIS | A 705 | 25.726 | 65.686 | −41.504 | 1.00 | 0.00 | N |
| ATOM | 5547 | CA | HIS | A 705 | 25.941 | 66.633 | −40.417 | 1.00 | 0.00 | C |
| ATOM | 5548 | C | HIS | A 705 | 25.131 | 66.183 | −39.209 | 1.00 | 0.00 | C |
| ATOM | 5549 | O | HIS | A 705 | 23.948 | 66.493 | −39.093 | 1.00 | 0.00 | O |
| ATOM | 5550 | CB | HIS | A 705 | 25.498 | 68.034 | −40.843 | 1.00 | 0.00 | C |
| ATOM | 5551 | CG | HIS | A 705 | 26.334 | 68.625 | −41.935 | 1.00 | 0.00 | C |
| ATOM | 5552 | ND1 | HIS | A 705 | 27.666 | 68.935 | −41.767 | 1.00 | 0.00 | N |
| ATOM | 5553 | CD2 | HIS | A 705 | 26.032 | 68.946 | −43.215 | 1.00 | 0.00 | C |
| ATOM | 5554 | CE1 | HIS | A 705 | 28.150 | 69.420 | −42.896 | 1.00 | 0.00 | C |
| ATOM | 5555 | NE2 | HIS | A 705 | 27.178 | 69.437 | −43.791 | 1.00 | 0.00 | N |
| ATOM | 5556 | N | VAL | A 706 | 25.775 | 65.446 | −38.312 | 1.00 | 0.00 | N |
| ATOM | 5557 | CA | VAL | A 706 | 25.106 | 64.947 | −37.119 | 1.00 | 0.00 | C |
| ATOM | 5558 | C | VAL | A 706 | 24.757 | 66.073 | −36.153 | 1.00 | 0.00 | C |
| ATOM | 5559 | O | VAL | A 706 | 25.624 | 66.842 | −35.747 | 1.00 | 0.00 | O |
| ATOM | 5560 | CB | VAL | A 706 | 25.993 | 63.918 | −36.385 | 1.00 | 0.00 | C |
| ATOM | 5561 | CG1 | VAL | A 706 | 25.277 | 63.399 | −35.150 | 1.00 | 0.00 | C |
| ATOM | 5562 | CG2 | VAL | A 706 | 26.336 | 62.774 | −37.322 | 1.00 | 0.00 | C |
| ATOM | 5563 | N | PRO | A 707 | 23.473 | 66.187 | −35.778 | 1.00 | 0.00 | N |
| ATOM | 5564 | CA | PRO | A 707 | 23.024 | 67.230 | −34.852 | 1.00 | 0.00 | C |
| ATOM | 5565 | C | PRO | A 707 | 23.628 | 67.088 | −33.453 | 1.00 | 0.00 | C |
| ATOM | 5566 | O | PRO | A 707 | 23.377 | 66.107 | −32.753 | 1.00 | 0.00 | O |
| ATOM | 5567 | CB | PRO | A 707 | 21.506 | 67.047 | −34.834 | 1.00 | 0.00 | C |
| ATOM | 5568 | CG | PRO | A 707 | 21.214 | 66.477 | −36.190 | 1.00 | 0.00 | C |
| ATOM | 5569 | CD | PRO | A 707 | 22.320 | 65.466 | −36.346 | 1.00 | 0.00 | C |
| ATOM | 5570 | N | VAL | A 708 | 24.434 | 68.069 | −33.063 | 1.00 | 0.00 | N |
| ATOM | 5571 | CA | VAL | A 708 | 25.068 | 68.098 | −31.746 | 1.00 | 0.00 | C |
| ATOM | 5572 | C | VAL | A 708 | 25.046 | 69.568 | −31.350 | 1.00 | 0.00 | C |
| ATOM | 5573 | O | VAL | A 708 | 25.821 | 70.369 | −31.865 | 1.00 | 0.00 | O |
| ATOM | 5574 | CB | VAL | A 708 | 26.529 | 67.592 | −31.802 | 1.00 | 0.00 | C |
| ATOM | 5575 | CG1 | VAL | A 708 | 27.164 | 67.665 | −30.415 | 1.00 | 0.00 | C |
| ATOM | 5576 | CG2 | VAL | A 708 | 26.561 | 66.162 | −32.317 | 1.00 | 0.00 | C |
| ATOM | 5577 | N | HIS | A 709 | 24.141 | 69.922 | −30.447 | 1.00 | 0.00 | N |
| ATOM | 5578 | CA | HIS | A 709 | 23.999 | 71.310 | −30.034 | 1.00 | 0.00 | C |
| ATOM | 5579 | C | HIS | A 709 | 24.441 | 71.582 | −28.606 | 1.00 | 0.00 | C |
| ATOM | 5580 | O | HIS | A 709 | 24.063 | 70.864 | −27.681 | 1.00 | 0.00 | O |
| ATOM | 5581 | CB | HIS | A 709 | 22.539 | 71.753 | −30.183 | 1.00 | 0.00 | C |
| ATOM | 5582 | CG | HIS | A 709 | 22.007 | 71.652 | −31.578 | 1.00 | 0.00 | C |
| ATOM | 5583 | ND1 | HIS | A 709 | 21.637 | 72.758 | −32.316 | 1.00 | 0.00 | N |
| ATOM | 5584 | CD2 | HIS | A 709 | 21.754 | 70.577 | −32.362 | 1.00 | 0.00 | C |
| ATOM | 5585 | CE1 | HIS | A 709 | 21.177 | 72.367 | −33.491 | 1.00 | 0.00 | C |
| ATOM | 5586 | NE2 | HIS | A 709 | 21.237 | 71.049 | −33.545 | 1.00 | 0.00 | N |
| ATOM | 5587 | N | PHE | A 710 | 25.247 | 72.626 | −28.441 | 1.00 | 0.00 | N |
| ATOM | 5588 | CA | PHE | A 710 | 25.708 | 73.044 | −27.125 | 1.00 | 0.00 | C |
| ATOM | 5589 | C | PHE | A 710 | 24.733 | 74.129 | −26.682 | 1.00 | 0.00 | C |
| ATOM | 5590 | O | PHE | A 710 | 24.297 | 74.961 | −27.491 | 1.00 | 0.00 | O |
| ATOM | 5591 | CB | PHE | A 710 | 27.127 | 73.621 | −27.198 | 1.00 | 0.00 | C |
| ATOM | 5592 | CG | PHE | A 710 | 28.206 | 72.677 | −26.719 | 1.00 | 0.00 | C |
| ATOM | 5593 | CD1 | PHE | A 710 | 29.372 | 72.502 | −27.460 | 1.00 | 0.00 | C |
| ATOM | 5594 | CD2 | PHE | A 710 | 28.075 | 71.996 | −25.511 | 1.00 | 0.00 | C |
| ATOM | 5595 | CE1 | PHE | A 710 | 30.393 | 71.665 | −27.004 | 1.00 | 0.00 | C |
| ATOM | 5596 | CE2 | PHE | A 710 | 29.090 | 71.157 | −25.046 | 1.00 | 0.00 | C |
| ATOM | 5597 | CZ | PHE | A 710 | 30.249 | 70.992 | −25.794 | 1.00 | 0.00 | C |
| ATOM | 5598 | N | LYS | A 711 | 24.386 | 74.114 | −25.403 | 1.00 | 0.00 | N |
| ATOM | 5599 | CA | LYS | A 711 | 23.460 | 75.090 | −24.854 | 1.00 | 0.00 | C |
| ATOM | 5600 | C | LYS | A 711 | 23.782 | 75.306 | −23.385 | 1.00 | 0.00 | C |
| ATOM | 5601 | O | LYS | A 711 | 24.162 | 74.369 | −22.683 | 1.00 | 0.00 | O |
| ATOM | 5602 | CB | LYS | A 711 | 22.022 | 74.581 | −25.003 | 1.00 | 0.00 | C |
| ATOM | 5603 | CG | LYS | A 711 | 20.971 | 75.484 | −24.386 | 1.00 | 0.00 | C |
| ATOM | 5604 | CD | LYS | A 711 | 19.576 | 74.871 | −24.505 | 1.00 | 0.00 | C |
| ATOM | 5605 | CE | LYS | A 711 | 19.139 | 74.740 | −25.956 | 1.00 | 0.00 | C |
| ATOM | 5606 | NZ | LYS | A 711 | 17.802 | 74.087 | −26.064 | 1.00 | 0.00 | N |
| ATOM | 5607 | N | PHE | A 712 | 23.638 | 76.543 | −22.924 | 1.00 | 0.00 | N |
| ATOM | 5608 | CA | PHE | A 712 | 23.899 | 76.859 | −21.530 | 1.00 | 0.00 | C |
| ATOM | 5609 | C | PHE | A 712 | 22.590 | 77.208 | −20.843 | 1.00 | 0.00 | C |
| ATOM | 5610 | O | PHE | A 712 | 21.803 | 78.000 | −21.361 | 1.00 | 0.00 | O |
| ATOM | 5611 | CB | PHE | A 712 | 24.890 | 78.025 | −21.412 | 1.00 | 0.00 | C |
| ATOM | 5612 | CG | PHE | A 712 | 26.310 | 77.643 | −21.723 | 1.00 | 0.00 | C |
| ATOM | 5613 | CD1 | PHE | A 712 | 26.792 | 77.687 | −23.028 | 1.00 | 0.00 | C |
| ATOM | 5614 | CD2 | PHE | A 712 | 27.153 | 77.194 | −20.712 | 1.00 | 0.00 | C |
| ATOM | 5615 | CE1 | PHE | A 712 | 28.095 | 77.287 | −23.321 | 1.00 | 0.00 | C |
| ATOM | 5616 | CE2 | PHE | A 712 | 28.457 | 76.791 | −20.992 | 1.00 | 0.00 | C |
| ATOM | 5617 | CZ | PHE | A 712 | 28.929 | 76.836 | −22.298 | 1.00 | 0.00 | C |
| ATOM | 5618 | N | LEU | A 713 | 22.346 | 76.594 | −19.690 | 1.00 | 0.00 | N |
| ATOM | 5619 | CA | LEU | A 713 | 21.122 | 76.856 | −18.945 | 1.00 | 0.00 | C |
| ATOM | 5620 | C | LEU | A 713 | 21.452 | 77.152 | −17.492 | 1.00 | 0.00 | C |
| ATOM | 5621 | O | LEU | A 713 | 22.615 | 77.103 | −17.087 | 1.00 | 0.00 | O |
| ATOM | 5622 | CB | LEU | A 713 | 20.164 | 75.657 | −19.033 | 1.00 | 0.00 | C |
| ATOM | 5623 | CG | LEU | A 713 | 19.730 | 75.212 | −20.434 | 1.00 | 0.00 | C |
| ATOM | 5624 | CD1 | LEU | A 713 | 20.690 | 74.148 | −20.956 | 1.00 | 0.00 | C |
| ATOM | 5625 | CD2 | LEU | A 713 | 18.311 | 74.644 | −20.385 | 1.00 | 0.00 | C |
| ATOM | 5626 | N | LYS | A 714 | 20.434 | 77.476 | −16.706 | 1.00 | 0.00 | N |
| ATOM | 5627 | CA | LYS | A 714 | 20.651 | 77.773 | −15.304 | 1.00 | 0.00 | C |
| ATOM | 5628 | C | LYS | A 714 | 19.588 | 77.169 | −14.402 | 1.00 | 0.00 | C |
| ATOM | 5629 | O | LYS | A 714 | 18.402 | 77.128 | −14.748 | 1.00 | 0.00 | O |
| ATOM | 5630 | CB | LYS | A 714 | 20.710 | 79.289 | −15.082 | 1.00 | 0.00 | C |
| ATOM | 5631 | CG | LYS | A 714 | 19.516 | 80.053 | −15.625 | 1.00 | 0.00 | C |
| ATOM | 5632 | CD | LYS | A 714 | 19.541 | 81.521 | −15.191 | 1.00 | 0.00 | C |
| ATOM | 5633 | CE | LYS | A 714 | 20.781 | 82.248 | −15.699 | 1.00 | 0.00 | C |
| ATOM | 5634 | NZ | LYS | A 714 | 20.828 | 83.671 | −15.245 | 1.00 | 0.00 | N |
| ATOM | 5635 | N | TYR | A 715 | 20.037 | 76.675 | −13.251 | 1.00 | 0.00 | N |
| ATOM | 5636 | CA | TYR | A 715 | 19.156 | 76.099 | −12.249 | 1.00 | 0.00 | C |
| ATOM | 5637 | C | TYR | A 715 | 19.067 | 77.134 | −11.140 | 1.00 | 0.00 | C |
| ATOM | 5638 | O | TYR | A 715 | 20.017 | 77.886 | −10.912 | 1.00 | 0.00 | O |
| ATOM | 5639 | CB | TYR | A 715 | 19.740 | 74.809 | −11.654 | 1.00 | 0.00 | C |
| ATOM | 5640 | CG | TYR | A 715 | 19.645 | 73.587 | −12.537 | 1.00 | 0.00 | C |
| ATOM | 5641 | CD1 | TYR | A 715 | 20.722 | 73.181 | −13.324 | 1.00 | 0.00 | C |
| ATOM | 5642 | CD2 | TYR | A 715 | 18.476 | 72.826 | −12.576 | 1.00 | 0.00 | C |
| ATOM | 5643 | CE1 | TYR | A 715 | 20.639 | 72.037 | −14.130 | 1.00 | 0.00 | C |
| ATOM | 5644 | CE2 | TYR | A 715 | 18.382 | 71.689 | −13.376 | 1.00 | 0.00 | C |
| ATOM | 5645 | CZ | TYR | A 715 | 19.463 | 71.299 | −14.148 | 1.00 | 0.00 | C |
| ATOM | 5646 | OH | TYR | A 715 | 19.364 | 70.170 | −14.932 | 1.00 | 0.00 | O |
| ATOM | 5647 | N | GLY | A 716 | 17.936 | 77.170 | −10.449 | 1.00 | 0.00 | N |
| ATOM | 5648 | CA | GLY | A 716 | 17.779 | 78.116 | −9.363 | 1.00 | 0.00 | C |
| ATOM | 5649 | C | GLY | A 716 | 17.683 | 77.391 | −8.036 | 1.00 | 0.00 | C |
| ATOM | 5650 | O | GLY | A 716 | 18.004 | 76.202 | −7.937 | 1.00 | 0.00 | O |
| ATOM | 5651 | N | VAL | A 717 | 17.249 | 78.109 | −7.009 | 1.00 | 0.00 | N |
| ATOM | 5652 | CA | VAL | A 717 | 17.097 | 77.535 | −5.681 | 1.00 | 0.00 | C |
| ATOM | 5653 | C | VAL | A 717 | 15.641 | 77.683 | −5.254 | 1.00 | 0.00 | C |
| ATOM | 5654 | O | VAL | A 717 | 14.960 | 78.619 | −5.669 | 1.00 | 0.00 | O |
| ATOM | 5655 | CB | VAL | A 717 | 18.014 | 78.246 | −4.667 | 1.00 | 0.00 | C |
| ATOM | 5656 | CG1 | VAL | A 717 | 17.799 | 77.677 | −3.280 | 1.00 | 0.00 | C |
| ATOM | 5657 | CG2 | VAL | A 717 | 19.475 | 78.077 | −5.089 | 1.00 | 0.00 | C |
| ATOM | 5658 | N | ARG | A 718 | 15.162 | 76.754 | −4.436 | 1.00 | 0.00 | N |
| ATOM | 5659 | CA | ARG | A 718 | 13.778 | 76.791 | −3.979 | 1.00 | 0.00 | C |
| ATOM | 5660 | C | ARG | A 718 | 13.525 | 77.925 | −2.995 | 1.00 | 0.00 | C |
| ATOM | 5661 | O | ARG | A 718 | 14.353 | 78.213 | −2.134 | 1.00 | 0.00 | O |
| ATOM | 5662 | CB | ARG | A 718 | 13.404 | 75.452 | −3.338 | 1.00 | 0.00 | C |
| ATOM | 5663 | CG | ARG | A 718 | 13.490 | 74.288 | −4.303 | 1.00 | 0.00 | C |
| ATOM | 5664 | CD | ARG | A 718 | 13.389 | 72.943 | −3.602 | 1.00 | 0.00 | C |
| ATOM | 5665 | NE | ARG | A 718 | 13.659 | 71.859 | −4.541 | 1.00 | 0.00 | N |
| ATOM | 5666 | CZ | ARG | A 718 | 13.687 | 70.570 | −4.222 | 1.00 | 0.00 | C |
| ATOM | 5667 | NH1 | ARG | A 718 | 13.458 | 70.182 | −2.973 | 1.00 | 0.00 | N |
| ATOM | 5668 | NH2 | ARG | A 718 | 13.948 | 69.667 | −5.158 | 1.00 | 0.00 | N |
| ATOM | 5669 | N | SER | A 719 | 12.373 | 78.569 | −3.132 | 1.00 | 0.00 | N |
| ATOM | 5670 | CA | SER | A 719 | 12.006 | 79.668 | −2.249 | 1.00 | 0.00 | C |
| ATOM | 5671 | C | SER | A 719 | 11.347 | 79.114 | −0.991 | 1.00 | 0.00 | C |
| ATOM | 5672 | O | SER | A 719 | 11.191 | 79.819 | 0.003 | 1.00 | 0.00 | O |
| ATOM | 5673 | CB | SER | A 719 | 11.044 | 80.618 | −2.965 | 1.00 | 0.00 | C |
| ATOM | 5674 | OG | SER | A 719 | 9.886 | 79.927 | −3.397 | 1.00 | 0.00 | O |
| ATOM | 5675 | N | HIS | A 720 | 10.965 | 77.841 | −1.048 | 1.00 | 0.00 | N |
| ATOM | 5676 | CA | HIS | A 720 | 10.320 | 77.174 | 0.077 | 1.00 | 0.00 | C |
| ATOM | 5677 | C | HIS | A 720 | 11.030 | 75.865 | 0.407 | 1.00 | 0.00 | C |
| ATOM | 5678 | O | HIS | A 720 | 11.457 | 75.139 | −0.489 | 1.00 | 0.00 | O |
| ATOM | 5679 | CB | HIS | A 720 | 8.857 | 76.872 | −0.256 | 1.00 | 0.00 | C |
| ATOM | 5680 | CG | HIS | A 720 | 8.080 | 78.069 | −0.705 | 1.00 | 0.00 | C |
| ATOM | 5681 | ND1 | HIS | A 720 | 7.945 | 79.202 | 0.068 | 1.00 | 0.00 | N |
| ATOM | 5682 | CD2 | HIS | A 720 | 7.398 | 78.310 | −1.850 | 1.00 | 0.00 | C |
| ATOM | 5683 | CE1 | HIS | A 720 | 7.213 | 80.090 | −0.581 | 1.00 | 0.00 | C |
| ATOM | 5684 | NE2 | HIS | A 720 | 6.868 | 79.574 | −1.748 | 1.00 | 0.00 | N |
| ATOM | 5685 | N | GLY | A 721 | 11.154 | 75.571 | 1.697 | 1.00 | 0.00 | N |
| ATOM | 5686 | CA | GLY | A 721 | 11.794 | 74.337 | 2.114 | 1.00 | 0.00 | C |
| ATOM | 5687 | C | GLY | A 721 | 13.311 | 74.356 | 2.111 | 1.00 | 0.00 | C |
| ATOM | 5688 | O | GLY | A 721 | 13.934 | 75.409 | 2.237 | 1.00 | 0.00 | O |
| ATOM | 5689 | N | ASP | A 722 | 13.899 | 73.173 | 1.957 | 1.00 | 0.00 | N |
| ATOM | 5690 | CA | ASP | A 722 | 15.348 | 72.999 | 1.950 | 1.00 | 0.00 | C |
| ATOM | 5691 | C | ASP | A 722 | 16.022 | 73.715 | 0.785 | 1.00 | 0.00 | C |
| ATOM | 5692 | O | ASP | A 722 | 15.582 | 73.611 | −0.358 | 1.00 | 0.00 | O |
| ATOM | 5693 | CB | ASP | A 722 | 15.681 | 71.505 | 1.904 | 1.00 | 0.00 | C |
| ATOM | 5694 | CG | ASP | A 722 | 15.137 | 70.750 | 3.109 | 1.00 | 0.00 | C |
| ATOM | 5695 | OD1 | ASP | A 722 | 15.062 | 69.503 | 3.053 | 1.00 | 0.00 | O |
| ATOM | 5696 | OD2 | ASP | A 722 | 14.792 | 71.406 | 4.116 | 1.00 | 0.00 | O |
| ATOM | 5697 | N | ARG | A 723 | 17.096 | 74.439 | 1.088 | 1.00 | 0.00 | N |
| ATOM | 5698 | CA | ARG | A 723 | 17.840 | 75.175 | 0.072 | 1.00 | 0.00 | C |
| ATOM | 5699 | C | ARG | A 723 | 19.143 | 74.485 | −0.312 | 1.00 | 0.00 | C |
| ATOM | 5700 | O | ARG | A 723 | 19.777 | 73.819 | 0.510 | 1.00 | 0.00 | O |
| ATOM | 5701 | CB | ARG | A 723 | 18.163 | 76.594 | 0.557 | 1.00 | 0.00 | C |
| ATOM | 5702 | CG | ARG | A 723 | 17.024 | 77.600 | 0.426 | 1.00 | 0.00 | C |
| ATOM | 5703 | CD | ARG | A 723 | 15.961 | 77.418 | 1.494 | 1.00 | 0.00 | C |
| ATOM | 5704 | NE | ARG | A 723 | 14.923 | 78.446 | 1.399 | 1.00 | 0.00 | N |
| ATOM | 5705 | CZ | ARG | A 723 | 13.945 | 78.612 | 2.286 | 1.00 | 0.00 | C |
| ATOM | 5706 | NH1 | ARG | A 723 | 13.860 | 77.817 | 3.346 | 1.00 | 0.00 | N |
| ATOM | 5707 | NH2 | ARG | A 723 | 13.052 | 79.580 | 2.116 | 1.00 | 0.00 | N |
| ATOM | 5708 | N | SER | A 724 | 19.536 | 74.657 | −1.570 | 1.00 | 0.00 | N |
| ATOM | 5709 | CA | SER | A 724 | 20.772 | 74.081 | −2.075 | 1.00 | 0.00 | C |
| ATOM | 5710 | C | SER | A 724 | 21.948 | 74.758 | −1.383 | 1.00 | 0.00 | C |
| ATOM | 5711 | O | SER | A 724 | 21.888 | 75.948 | −1.056 | 1.00 | 0.00 | O |
| ATOM | 5712 | CB | SER | A 724 | 20.882 | 74.300 | −3.586 | 1.00 | 0.00 | C |
| ATOM | 5713 | OG | SER | A 724 | 19.801 | 73.699 | −4.277 | 1.00 | 0.00 | O |
| ATOM | 5714 | N | GLY | A 725 | 23.015 | 73.996 | −1.166 | 1.00 | 0.00 | N |
| ATOM | 5715 | CA | GLY | A 725 | 24.209 | 74.528 | −0.527 | 1.00 | 0.00 | C |
| ATOM | 5716 | C | GLY | A 725 | 25.431 | 73.840 | −1.110 | 1.00 | 0.00 | C |
| ATOM | 5717 | O | GLY | A 725 | 25.356 | 73.275 | −2.202 | 1.00 | 0.00 | O |
| ATOM | 5718 | N | ALA | A 726 | 26.550 | 73.871 | −0.391 | 1.00 | 0.00 | N |
| ATOM | 5719 | CA | ALA | A 726 | 27.780 | 73.243 | −0.867 | 1.00 | 0.00 | C |
| ATOM | 5720 | C | ALA | A 726 | 27.626 | 71.737 | −1.104 | 1.00 | 0.00 | C |
| ATOM | 5721 | O | ALA | A 726 | 28.261 | 71.175 | −2.000 | 1.00 | 0.00 | O |
| ATOM | 5722 | CB | ALA | A 726 | 28.917 | 73.496 | 0.131 | 1.00 | 0.00 | C |
| ATOM | 5723 | N | TYR | A 727 | 26.785 | 71.088 | −0.304 | 1.00 | 0.00 | N |
| ATOM | 5724 | CA | TYR | A 727 | 26.581 | 69.646 | −0.436 | 1.00 | 0.00 | C |
| ATOM | 5725 | C | TYR | A 727 | 25.375 | 69.273 | −1.286 | 1.00 | 0.00 | C |
| ATOM | 5726 | O | TYR | A 727 | 25.474 | 68.466 | −2.213 | 1.00 | 0.00 | O |
| ATOM | 5727 | CB | TYR | A 727 | 26.377 | 68.994 | 0.935 | 1.00 | 0.00 | C |
| ATOM | 5728 | CG | TYR | A 727 | 27.394 | 69.345 | 1.985 | 1.00 | 0.00 | C |
| ATOM | 5729 | CD1 | TYR | A 727 | 27.235 | 70.472 | 2.793 | 1.00 | 0.00 | C |
| ATOM | 5730 | CD2 | TYR | A 727 | 28.514 | 68.541 | 2.186 | 1.00 | 0.00 | C |
| ATOM | 5731 | CE1 | TYR | A 727 | 28.168 | 70.783 | 3.783 | 1.00 | 0.00 | C |
| ATOM | 5732 | CE2 | TYR | A 727 | 29.450 | 68.842 | 3.165 | 1.00 | 0.00 | C |
| ATOM | 5733 | CZ | TYR | A 727 | 29.274 | 69.959 | 3.962 | 1.00 | 0.00 | C |
| ATOM | 5734 | OH | TYR | A 727 | 30.185 | 70.230 | 4.951 | 1.00 | 0.00 | O |
| ATOM | 5735 | N | LEU | A 728 | 24.234 | 69.864 | −0.946 | 1.00 | 0.00 | N |
| ATOM | 5736 | CA | LEU | A 728 | 22.971 | 69.565 | −1.608 | 1.00 | 0.00 | C |
| ATOM | 5737 | C | LEU | A 728 | 22.621 | 70.324 | −2.881 | 1.00 | 0.00 | C |
| ATOM | 5738 | O | LEU | A 728 | 22.860 | 71.525 | −2.999 | 1.00 | 0.00 | O |
| ATOM | 5739 | CB | LEU | A 728 | 21.821 | 69.753 | −0.611 | 1.00 | 0.00 | C |
| ATOM | 5740 | CG | LEU | A 728 | 21.986 | 69.104 | 0.766 | 1.00 | 0.00 | C |
| ATOM | 5741 | CD1 | LEU | A 728 | 20.725 | 69.327 | 1.599 | 1.00 | 0.00 | C |
| ATOM | 5742 | CD2 | LEU | A 728 | 22.265 | 67.623 | 0.598 | 1.00 | 0.00 | C |
| ATOM | 5743 | N | PHE | A 729 | 22.041 | 69.590 | −3.828 | 1.00 | 0.00 | N |
| ATOM | 5744 | CA | PHE | A 729 | 21.571 | 70.146 | −5.089 | 1.00 | 0.00 | C |
| ATOM | 5745 | C | PHE | A 729 | 20.050 | 70.000 | −4.996 | 1.00 | 0.00 | C |
| ATOM | 5746 | O | PHE | A 729 | 19.521 | 68.892 | −5.082 | 1.00 | 0.00 | O |
| ATOM | 5747 | CB | PHE | A 729 | 22.097 | 69.335 | −6.279 | 1.00 | 0.00 | C |
| ATOM | 5748 | CG | PHE | A 729 | 21.637 | 69.848 | −7.624 | 1.00 | 0.00 | C |
| ATOM | 5749 | CD1 | PHE | A 729 | 21.579 | 68.995 | −8.721 | 1.00 | 0.00 | C |
| ATOM | 5750 | CD2 | PHE | A 729 | 21.283 | 71.187 | −7.797 | 1.00 | 0.00 | C |
| ATOM | 5751 | CE1 | PHE | A 729 | 21.173 | 69.463 | −9.974 | 1.00 | 0.00 | C |
| ATOM | 5752 | CE2 | PHE | A 729 | 20.877 | 71.666 | −9.045 | 1.00 | 0.00 | C |
| ATOM | 5753 | CZ | PHE | A 729 | 20.822 | 70.804 | −10.134 | 1.00 | 0.00 | C |
| ATOM | 5754 | N | LEU | A 730 | 19.356 | 71.117 | −4.802 | 1.00 | 0.00 | N |
| ATOM | 5755 | CA | LEU | A 730 | 17.901 | 71.115 | −4.680 | 1.00 | 0.00 | C |
| ATOM | 5756 | C | LEU | A 730 | 17.326 | 72.146 | −5.636 | 1.00 | 0.00 | C |
| ATOM | 5757 | O | LEU | A 730 | 16.852 | 73.201 | −5.215 | 1.00 | 0.00 | O |
| ATOM | 5758 | CB | LEU | A 730 | 17.506 | 71.459 | −3.245 | 1.00 | 0.00 | C |
| ATOM | 5759 | CG | LEU | A 730 | 17.893 | 70.418 | −2.192 | 1.00 | 0.00 | C |
| ATOM | 5760 | CD1 | LEU | A 730 | 17.864 | 71.040 | −0.809 | 1.00 | 0.00 | C |
| ATOM | 5761 | CD2 | LEU | A 730 | 16.942 | 69.232 | −2.277 | 1.00 | 0.00 | C |
| ATOM | 5762 | N | PRO | A 731 | 17.355 | 71.845 | −6.942 | 1.00 | 0.00 | N |
| ATOM | 5763 | CA | PRO | A 731 | 16.844 | 72.754 | −7.968 | 1.00 | 0.00 | C |
| ATOM | 5764 | C | PRO | A 731 | 15.358 | 73.063 | −7.877 | 1.00 | 0.00 | C |
| ATOM | 5765 | O | PRO | A 731 | 14.559 | 72.233 | −7.434 | 1.00 | 0.00 | O |
| ATOM | 5766 | CB | PRO | A 731 | 17.198 | 72.036 | −9.262 | 1.00 | 0.00 | C |
| ATOM | 5767 | CG | PRO | A 731 | 17.026 | 70.593 | −8.879 | 1.00 | 0.00 | C |
| ATOM | 5768 | CD | PRO | A 731 | 17.699 | 70.535 | −7.528 | 1.00 | 0.00 | C |
| ATOM | 5769 | N | ASN | A 732 | 14.998 | 74.271 | −8.297 | 1.00 | 0.00 | N |
| ATOM | 5770 | CA | ASN | A 732 | 13.605 | 74.687 | −8.300 | 1.00 | 0.00 | C |
| ATOM | 5771 | C | ASN | A 732 | 13.053 | 74.332 | −9.674 | 1.00 | 0.00 | C |
| ATOM | 5772 | O | ASN | A 732 | 12.525 | 75.182 | −10.392 | 1.00 | 0.00 | O |
| ATOM | 5773 | CB | ASN | A 732 | 13.493 | 76.194 | −8.034 | 1.00 | 0.00 | C |
| ATOM | 5774 | CG | ASN | A 732 | 14.176 | 77.033 | −9.096 | 1.00 | 0.00 | C |
| ATOM | 5775 | OD1 | ASN | A 732 | 15.263 | 76.700 | −9.563 | 1.00 | 0.00 | O |
| ATOM | 5776 | ND2 | ASN | A 732 | 13.544 | 78.140 | −9.473 | 1.00 | 0.00 | N |
| ATOM | 5777 | N | GLY | A 733 | 13.204 | 73.059 | −10.035 | 1.00 | 0.00 | N |
| ATOM | 5778 | CA | GLY | A 733 | 12.718 | 72.577 | −11.315 | 1.00 | 0.00 | C |
| ATOM | 5779 | C | GLY | A 733 | 13.788 | 72.479 | −12.384 | 1.00 | 0.00 | C |
| ATOM | 5780 | O | GLY | A 733 | 14.952 | 72.810 | −12.133 | 1.00 | 0.00 | O |
| ATOM | 5781 | N | PRO | A 734 | 13.424 | 72.017 | −13.594 | 1.00 | 0.00 | N |
| ATOM | 5782 | CA | PRO | A 734 | 14.363 | 71.880 | −14.710 | 1.00 | 0.00 | C |
| ATOM | 5783 | C | PRO | A 734 | 15.038 | 73.216 | −14.995 | 1.00 | 0.00 | C |
| ATOM | 5784 | O | PRO | A 734 | 14.477 | 74.274 | −14.709 | 1.00 | 0.00 | O |
| ATOM | 5785 | CB | PRO | A 734 | 13.469 | 71.439 | −15.867 | 1.00 | 0.00 | C |
| ATOM | 5786 | CG | PRO | A 734 | 12.373 | 70.695 | −15.187 | 1.00 | 0.00 | C |
| ATOM | 5787 | CD | PRO | A 734 | 12.078 | 71.569 | −13.991 | 1.00 | 0.00 | C |
| ATOM | 5788 | N | ALA | A 735 | 16.234 | 73.160 | −15.570 | 1.00 | 0.00 | N |
| ATOM | 5789 | CA | ALA | A 735 | 16.992 | 74.365 | −15.883 | 1.00 | 0.00 | C |
| ATOM | 5790 | C | ALA | A 735 | 16.302 | 75.227 | −16.938 | 1.00 | 0.00 | C |
| ATOM | 5791 | O | ALA | A 735 | 15.535 | 74.728 | −17.759 | 1.00 | 0.00 | O |
| ATOM | 5792 | CB | ALA | A 735 | 18.391 | 73.988 | −16.355 | 1.00 | 0.00 | C |
| ATOM | 5793 | N | SER | A 736 | 16.585 | 76.526 | −16.896 | 1.00 | 0.00 | N |
| ATOM | 5794 | CA | SER | A 736 | 16.025 | 77.487 | −17.839 | 1.00 | 0.00 | C |
| ATOM | 5795 | C | SER | A 736 | 17.169 | 77.998 | −18.709 | 1.00 | 0.00 | C |
| ATOM | 5796 | O | SER | A 736 | 18.277 | 78.210 | −18.222 | 1.00 | 0.00 | O |
| ATOM | 5797 | CB | SER | A 736 | 15.392 | 78.662 | −17.090 | 1.00 | 0.00 | C |
| ATOM | 5798 | OG | SER | A 736 | 14.416 | 78.216 | −16.165 | 1.00 | 0.00 | O |
| ATOM | 5799 | N | PRO | A 737 | 16.912 | 78.213 | −20.007 | 1.00 | 0.00 | N |
| ATOM | 5800 | CA | PRO | A 737 | 17.957 | 78.701 | −20.913 | 1.00 | 0.00 | C |
| ATOM | 5801 | C | PRO | A 737 | 18.540 | 80.043 | −20.481 | 1.00 | 0.00 | C |
| ATOM | 5802 | O | PRO | A 737 | 17.818 | 80.915 | −20.001 | 1.00 | 0.00 | O |
| ATOM | 5803 | CB | PRO | A 737 | 17.231 | 78.808 | −22.255 | 1.00 | 0.00 | C |
| ATOM | 5804 | CG | PRO | A 737 | 16.131 | 77.793 | −22.141 | 1.00 | 0.00 | C |
| ATOM | 5805 | CD | PRO | A 737 | 15.647 | 78.009 | −20.731 | 1.00 | 0.00 | C |
| ATOM | 5806 | N | VAL | A 738 | 19.851 | 80.203 | −20.639 | 1.00 | 0.00 | N |
| ATOM | 5807 | CA | VAL | A 738 | 20.496 | 81.465 | −20.297 | 1.00 | 0.00 | C |
| ATOM | 5808 | C | VAL | A 738 | 20.181 | 82.434 | −21.438 | 1.00 | 0.00 | C |
| ATOM | 5809 | O | VAL | A 738 | 20.315 | 82.078 | −22.607 | 1.00 | 0.00 | O |
| ATOM | 5810 | CB | VAL | A 738 | 22.038 | 81.312 | −20.188 | 1.00 | 0.00 | C |
| ATOM | 5811 | CG1 | VAL | A 738 | 22.697 | 82.689 | −20.098 | 1.00 | 0.00 | C |
| ATOM | 5812 | CG2 | VAL | A 738 | 22.400 | 80.481 | −18.964 | 1.00 | 0.00 | C |
| ATOM | 5813 | N | GLU | A 739 | 19.742 | 83.643 | −21.100 | 1.00 | 0.00 | N |
| ATOM | 5814 | CA | GLU | A 739 | 19.429 | 84.648 | −22.113 | 1.00 | 0.00 | C |
| ATOM | 5815 | C | GLU | A 739 | 20.755 | 85.126 | −22.684 | 1.00 | 0.00 | C |
| ATOM | 5816 | O | GLU | A 739 | 21.545 | 85.761 | −21.988 | 1.00 | 0.00 | O |
| ATOM | 5817 | CB | GLU | A 739 | 18.670 | 85.818 | −21.487 | 1.00 | 0.00 | C |
| ATOM | 5818 | CG | GLU | A 739 | 17.304 | 85.436 | −20.946 | 1.00 | 0.00 | C |
| ATOM | 5819 | CD | GLU | A 739 | 16.357 | 84.965 | −22.035 | 1.00 | 0.00 | C |
| ATOM | 5820 | OE1 | GLU | A 739 | 15.224 | 84.555 | −21.701 | 1.00 | 0.00 | O |
| ATOM | 5821 | OE2 | GLU | A 739 | 16.741 | 85.010 | −23.224 | 1.00 | 0.00 | O |
| ATOM | 5822 | N | LEU | A 740 | 20.986 | 84.819 | −23.956 | 1.00 | 0.00 | N |
| ATOM | 5823 | CA | LEU | A 740 | 22.239 | 85.161 | −24.618 | 1.00 | 0.00 | C |
| ATOM | 5824 | C | LEU | A 740 | 22.318 | 86.525 | −25.288 | 1.00 | 0.00 | C |
| ATOM | 5825 | O | LEU | A 740 | 23.413 | 87.052 | −25.483 | 1.00 | 0.00 | O |
| ATOM | 5826 | CB | LEU | A 740 | 22.570 | 84.091 | −25.655 | 1.00 | 0.00 | C |
| ATOM | 5827 | CG | LEU | A 740 | 22.540 | 82.644 | −25.163 | 1.00 | 0.00 | C |
| ATOM | 5828 | CD1 | LEU | A 740 | 22.919 | 81.724 | −26.310 | 1.00 | 0.00 | C |
| ATOM | 5829 | CD2 | LEU | A 740 | 23.496 | 82.471 | −23.985 | 1.00 | 0.00 | C |
| ATOM | 5830 | N | GLY | A 741 | 21.172 | 87.094 | −25.645 | 1.00 | 0.00 | N |
| ATOM | 5831 | CA | GLY | A 741 | 21.187 | 88.379 | −26.319 | 1.00 | 0.00 | C |
| ATOM | 5832 | C | GLY | A 741 | 21.861 | 88.201 | −27.667 | 1.00 | 0.00 | C |
| ATOM | 5833 | O | GLY | A 741 | 21.617 | 87.214 | −28.356 | 1.00 | 0.00 | O |
| ATOM | 5834 | N | GLN | A 742 | 22.708 | 89.151 | −28.048 | 1.00 | 0.00 | N |
| ATOM | 5835 | CA | GLN | A 742 | 23.427 | 89.074 | −29.317 | 1.00 | 0.00 | C |
| ATOM | 5836 | C | GLN | A 742 | 24.920 | 89.094 | −28.991 | 1.00 | 0.00 | C |
| ATOM | 5837 | O | GLN | A 742 | 25.588 | 90.116 | −29.137 | 1.00 | 0.00 | O |
| ATOM | 5838 | CB | GLN | A 742 | 23.050 | 90.261 | −30.210 | 1.00 | 0.00 | C |
| ATOM | 5839 | CG | GLN | A 742 | 21.556 | 90.328 | −30.530 | 1.00 | 0.00 | C |
| ATOM | 5840 | CD | GLN | A 742 | 21.187 | 91.496 | −31.429 | 1.00 | 0.00 | C |
| ATOM | 5841 | OE1 | GLN | A 742 | 21.650 | 91.593 | −32.568 | 1.00 | 0.00 | O |
| ATOM | 5842 | NE2 | GLN | A 742 | 20.344 | 92.389 | −30.921 | 1.00 | 0.00 | N |
| ATOM | 5843 | N | PRO | A 743 | 25.458 | 87.948 | −28.545 | 1.00 | 0.00 | N |
| ATOM | 5844 | CA | PRO | A 743 | 26.868 | 87.789 | −28.175 | 1.00 | 0.00 | C |
| ATOM | 5845 | C | PRO | A 743 | 27.887 | 88.002 | −29.289 | 1.00 | 0.00 | C |
| ATOM | 5846 | O | PRO | A 743 | 27.612 | 87.745 | −30.461 | 1.00 | 0.00 | O |
| ATOM | 5847 | CB | PRO | A 743 | 26.912 | 86.371 | −27.616 | 1.00 | 0.00 | C |
| ATOM | 5848 | CG | PRO | A 743 | 25.904 | 85.660 | −28.464 | 1.00 | 0.00 | C |
| ATOM | 5849 | CD | PRO | A 743 | 24.758 | 86.649 | −28.496 | 1.00 | 0.00 | C |
| ATOM | 5850 | N | VAL | A 744 | 29.068 | 88.474 | −28.902 | 1.00 | 0.00 | N |
| ATOM | 5851 | CA | VAL | A 744 | 30.149 | 88.707 | −29.845 | 1.00 | 0.00 | C |
| ATOM | 5852 | C | VAL | A 744 | 30.876 | 87.386 | −30.076 | 1.00 | 0.00 | C |
| ATOM | 5853 | O | VAL | A 744 | 31.255 | 86.703 | −29.124 | 1.00 | 0.00 | O |
| ATOM | 5854 | CB | VAL | A 744 | 31.150 | 89.748 | −29.305 | 1.00 | 0.00 | C |
| ATOM | 5855 | CG1 | VAL | A 744 | 32.314 | 89.895 | −20.272 | 1.00 | 0.00 | C |
| ATOM | 5856 | CG2 | VAL | A 744 | 30.448 | 91.089 | −29.109 | 1.00 | 0.00 | C |
| ATOM | 5857 | N | VAL | A 745 | 31.055 | 87.030 | −31.343 | 1.00 | 0.00 | N |
| ATOM | 5858 | CA | VAL | A 745 | 31.717 | 85.785 | −31.711 | 1.00 | 0.00 | C |
| ATOM | 5859 | C | VAL | A 745 | 33.033 | 86.036 | −32.432 | 1.00 | 0.00 | C |
| ATOM | 5860 | O | VAL | A 745 | 33.091 | 86.832 | −33.367 | 1.00 | 0.00 | O |
| ATOM | 5861 | CB | VAL | A 745 | 30.822 | 84.939 | −32.641 | 1.00 | 0.00 | C |
| ATOM | 5862 | CG1 | VAL | A 745 | 31.536 | 83.652 | −33.021 | 1.00 | 0.00 | C |
| ATOM | 5863 | CG2 | VAL | A 745 | 29.500 | 84.642 | −31.960 | 1.00 | 0.00 | C |
| ATOM | 5864 | N | LEU | A 746 | 34.088 | 85.350 | −31.997 | 1.00 | 0.00 | N |
| ATOM | 5865 | CA | LEU | A 746 | 35.400 | 85.496 | −32.617 | 1.00 | 0.00 | C |
| ATOM | 5866 | C | LEU | A 746 | 35.813 | 84.206 | −33.313 | 1.00 | 0.00 | C |
| ATOM | 5867 | O | LEU | A 746 | 35.915 | 83.153 | −32.682 | 1.00 | 0.00 | O |
| ATOM | 5868 | CB | LEU | A 746 | 36.458 | 85.861 | −31.572 | 1.00 | 0.00 | C |
| ATOM | 5869 | CG | LEU | A 746 | 37.909 | 85.872 | −32.079 | 1.00 | 0.00 | C |
| ATOM | 5870 | CD1 | LEU | A 746 | 38.102 | 86.971 | −33.119 | 1.00 | 0.00 | C |
| ATOM | 5871 | CD2 | LEU | A 746 | 38.850 | 86.092 | −30.903 | 1.00 | 0.00 | C |
| ATOM | 5872 | N | VAL | A 747 | 36.067 | 84.301 | −34.612 | 1.00 | 0.00 | N |
| ATOM | 5873 | CA | VAL | A 747 | 36.468 | 83.140 | −35.394 | 1.00 | 0.00 | C |
| ATOM | 5874 | C | VAL | A 747 | 37.912 | 83.266 | −35.857 | 1.00 | 0.00 | C |
| ATOM | 5875 | O | VAL | A 747 | 38.277 | 84.222 | −36.544 | 1.00 | 0.00 | O |
| ATOM | 5876 | CB | VAL | A 747 | 35.558 | 82.961 | −36.639 | 1.00 | 0.00 | C |
| ATOM | 5877 | CG1 | VAL | A 747 | 35.974 | 81.712 | −37.415 | 1.00 | 0.00 | C |
| ATOM | 5878 | CG2 | VAL | A 747 | 34.100 | 82.864 | −36.207 | 1.00 | 0.00 | C |
| ATOM | 5879 | N | THR | A 748 | 38.737 | 82.304 | −35.464 | 1.00 | 0.00 | N |
| ATOM | 5880 | CA | THR | A 748 | 40.136 | 82.295 | −35.859 | 1.00 | 0.00 | C |
| ATOM | 5881 | C | THR | A 748 | 40.358 | 81.044 | −36.697 | 1.00 | 0.00 | C |
| ATOM | 5882 | O | THR | A 748 | 40.121 | 79.927 | −36.232 | 1.00 | 0.00 | O |
| ATOM | 5883 | CB | THR | A 748 | 41.067 | 82.274 | −34.624 | 1.00 | 0.00 | C |
| ATOM | 5884 | OG1 | THR | A 748 | 40.874 | 83.475 | −33.865 | 1.00 | 0.00 | O |
| ATOM | 5885 | CG2 | THR | A 748 | 42.532 | 82.173 | −35.054 | 1.00 | 0.00 | C |
| ATOM | 5886 | N | LYS | A 749 | 40.802 | 81.232 | −37.935 | 1.00 | 0.00 | N |
| ATOM | 5887 | CA | LYS | A 749 | 41.034 | 80.108 | −38.828 | 1.00 | 0.00 | C |
| ATOM | 5888 | C | LYS | A 749 | 42.500 | 79.968 | −39.202 | 1.00 | 0.00 | C |
| ATOM | 5889 | O | LYS | A 749 | 43.097 | 80.875 | −39.790 | 1.00 | 0.00 | O |
| ATOM | 5890 | CB | LYS | A 749 | 40.192 | 80.262 | −40.096 | 1.00 | 0.00 | C |
| ATOM | 5891 | CG | LYS | A 749 | 40.408 | 79.145 | −41.104 | 1.00 | 0.00 | C |
| ATOM | 5892 | CD | LYS | A 749 | 39.527 | 79.315 | −42.329 | 1.00 | 0.00 | C |
| ATOM | 5893 | CE | LYS | A 749 | 39.778 | 78.195 | −43.328 | 1.00 | 0.00 | C |
| ATOM | 5894 | NZ | LYS | A 749 | 38.938 | 78.336 | −44.547 | 1.00 | 0.00 | N |
| ATOM | 5895 | N | GLY | A 750 | 43.072 | 78.819 | −38.861 | 1.00 | 0.00 | N |
| ATOM | 5896 | CA | GLY | A 750 | 44.468 | 78.568 | −39.164 | 1.00 | 0.00 | C |
| ATOM | 5897 | C | GLY | A 750 | 44.667 | 77.201 | −39.778 | 1.00 | 0.00 | C |
| ATOM | 5898 | O | GLY | A 750 | 43.767 | 76.356 | −39.742 | 1.00 | 0.00 | O |
| ATOM | 5899 | N | LYS | A 751 | 45.848 | 76.984 | −40.347 | 1.00 | 0.00 | N |
| ATOM | 5900 | CA | LYS | A 751 | 46.173 | 75.714 | −40.979 | 1.00 | 0.00 | C |
| ATOM | 5901 | C | LYS | A 751 | 46.322 | 74.594 | −39.953 | 1.00 | 0.00 | C |
| ATOM | 5902 | O | LYS | A 751 | 46.006 | 73.441 | −40.238 | 1.00 | 0.00 | O |
| ATOM | 5903 | CB | LYS | A 751 | 47.476 | 75.840 | −41.774 | 1.00 | 0.00 | C |
| ATOM | 5904 | CG | LYS | A 751 | 47.397 | 76.756 | −42.993 | 1.00 | 0.00 | C |
| ATOM | 5905 | CD | LYS | A 751 | 46.565 | 76.128 | −44.106 | 1.00 | 0.00 | C |
| ATOM | 5906 | CE | LYS | A 751 | 46.628 | 76.955 | −45.387 | 1.00 | 0.00 | C |
| ATOM | 5907 | NZ | LYS | A 751 | 45.820 | 76.342 | −46.483 | 1.00 | 0.00 | N |
| ATOM | 5908 | N | LEU | A 752 | 46.802 | 74.940 | −38.763 | 1.00 | 0.00 | N |
| ATOM | 5909 | CA | LEU | A 752 | 47.011 | 73.953 | −37.709 | 1.00 | 0.00 | C |
| ATOM | 5910 | C | LEU | A 752 | 45.951 | 72.997 | −36.615 | 1.00 | 0.00 | C |
| ATOM | 5911 | O | LEU | A 752 | 45.630 | 72.972 | −36.010 | 1.00 | 0.00 | O |
| ATOM | 5912 | CB | LEU | A 752 | 48.390 | 74.152 | −37.072 | 1.00 | 0.00 | C |
| ATOM | 5913 | CG | LEU | A 752 | 49.622 | 74.116 | −37.983 | 1.00 | 0.00 | C |
| ATOM | 5914 | CD1 | LEU | A 752 | 50.878 | 74.287 | −37.136 | 1.00 | 0.00 | C |
| ATOM | 5915 | CD2 | LEU | A 752 | 49.669 | 72.798 | −38.749 | 1.00 | 0.00 | C |
| ATOM | 5916 | N | GLU | A 753 | 45.410 | 75.182 | −36.360 | 1.00 | 0.00 | N |
| ATOM | 5917 | CA | GLU | A 753 | 44.405 | 75.341 | −35.320 | 1.00 | 0.00 | C |
| ATOM | 5918 | C | GLU | A 753 | 43.414 | 76.452 | −35.644 | 1.00 | 0.00 | C |
| ATOM | 5919 | O | GLU | A 753 | 43.802 | 77.555 | −36.033 | 1.00 | 0.00 | O |
| ATOM | 5920 | CB | GLU | A 753 | 45.086 | 75.656 | −33.984 | 1.00 | 0.00 | C |
| ATOM | 5921 | CG | GLU | A 753 | 44.131 | 75.781 | −32.801 | 1.00 | 0.00 | C |
| ATOM | 5922 | CD | GLU | A 753 | 44.827 | 76.254 | −31.534 | 1.00 | 0.00 | C |
| ATOM | 5923 | OE1 | GLU | A 753 | 45.166 | 77.451 | −31.449 | 1.00 | 0.00 | O |
| ATOM | 5924 | OE2 | GLU | A 753 | 45.040 | 75.425 | −30.624 | 1.00 | 0.00 | O |
| ATOM | 5925 | N | SER | A 754 | 42.132 | 76.151 | −35.483 | 1.00 | 0.00 | N |
| ATOM | 5926 | CA | SER | A 754 | 41.080 | 77.127 | −35.717 | 1.00 | 0.00 | C |
| ATOM | 5927 | C | SER | A 754 | 40.201 | 77.081 | −34.481 | 1.00 | 0.00 | C |
| ATOM | 5928 | O | SER | A 754 | 40.272 | 76.124 | −33.707 | 1.00 | 0.00 | O |
| ATOM | 5929 | CB | SER | A 754 | 40.258 | 76.753 | −36.951 | 1.00 | 0.00 | C |
| ATOM | 5930 | OG | SER | A 754 | 41.062 | 76.784 | −38.117 | 1.00 | 0.00 | O |
| ATOM | 5931 | N | SER | A 755 | 39.378 | 78.102 | −34.282 | 1.00 | 0.00 | N |
| ATOM | 5932 | CA | SER | A 755 | 38.507 | 78.109 | −33.119 | 1.00 | 0.00 | C |
| ATOM | 5933 | C | SER | A 755 | 37.377 | 79.114 | −33.231 | 1.00 | 0.00 | C |
| ATOM | 5934 | O | SER | A 755 | 37.423 | 80.046 | −34.038 | 1.00 | 0.00 | O |
| ATOM | 5935 | CB | SER | A 755 | 39.317 | 78.407 | −31.852 | 1.00 | 0.00 | C |
| ATOM | 5936 | OG | SER | A 755 | 39.813 | 79.734 | −31.868 | 1.00 | 0.00 | O |
| ATOM | 5937 | N | VAL | A 756 | 36.353 | 78.897 | −32.419 | 1.00 | 0.00 | N |
| ATOM | 5938 | CA | VAL | A 756 | 35.205 | 79.783 | −32.358 | 1.00 | 0.00 | C |
| ATOM | 5939 | C | VAL | A 756 | 35.018 | 80.082 | −30.877 | 1.00 | 0.00 | C |
| ATOM | 5940 | O | VAL | A 756 | 34.830 | 79.167 | −30.074 | 1.00 | 0.00 | O |
| ATOM | 5941 | CB | VAL | A 756 | 33.934 | 79.114 | −32.905 | 1.00 | 0.00 | C |
| ATOM | 5942 | CG1 | VAL | A 756 | 32.733 | 80.032 | −32.691 | 1.00 | 0.00 | C |
| ATOM | 5943 | CG2 | VAL | A 756 | 34.115 | 78.790 | −34.384 | 1.00 | 0.00 | C |
| ATOM | 5944 | N | SER | A 757 | 35.085 | 81.361 | −30.524 | 1.00 | 0.00 | N |
| ATOM | 5945 | CA | SER | A 757 | 34.937 | 81.789 | −29.139 | 1.00 | 0.00 | C |
| ATOM | 5946 | C | SER | A 757 | 33.814 | 82.810 | −29.032 | 1.00 | 0.00 | C |
| ATOM | 5947 | O | SER | A 757 | 33.704 | 83.696 | −29.873 | 1.00 | 0.00 | O |
| ATOM | 5948 | CB | SER | A 757 | 36.244 | 82.416 | −28.645 | 1.00 | 0.00 | C |
| ATOM | 5949 | OG | SER | A 757 | 37.342 | 81.544 | −28.869 | 1.00 | 0.00 | O |
| ATOM | 5950 | N | VAL | A 758 | 32.982 | 82.690 | −28.004 | 1.00 | 0.00 | N |
| ATOM | 5951 | CA | VAL | A 758 | 31.878 | 83.630 | −27.828 | 1.00 | 0.00 | C |
| ATOM | 5952 | C | VAL | A 758 | 31.737 | 84.071 | −26.373 | 1.00 | 0.00 | C |
| ATOM | 5953 | O | VAL | A 758 | 31.854 | 83.260 | −25.446 | 1.00 | 0.00 | O |
| ATOM | 5954 | CB | VAL | A 758 | 30.536 | 83.021 | −28.336 | 1.00 | 0.00 | C |
| ATOM | 5955 | CG1 | VAL | A 758 | 30.260 | 81.691 | −27.646 | 1.00 | 0.00 | C |
| ATOM | 5956 | CG2 | VAL | A 758 | 29.393 | 83.997 | −28.097 | 1.00 | 0.00 | C |
| ATOM | 5957 | N | GLY | A 759 | 31.513 | 85.369 | −26.184 | 1.00 | 0.00 | N |
| ATOM | 5958 | CA | GLY | A 759 | 31.354 | 85.915 | −24.851 | 1.00 | 0.00 | C |
| ATOM | 5959 | C | GLY | A 759 | 29.905 | 85.866 | −24.411 | 1.00 | 0.00 | C |
| ATOM | 5960 | O | GLY | A 759 | 29.104 | 86.743 | −24.744 | 1.00 | 0.00 | O |
| ATOM | 5961 | N | LEU | A 760 | 29.565 | 84.824 | −23.664 | 1.00 | 0.00 | N |
| ATOM | 5962 | CA | LEU | A 760 | 28.210 | 84.643 | −23.165 | 1.00 | 0.00 | C |
| ATOM | 5963 | C | LEU | A 760 | 28.151 | 85.121 | −21.723 | 1.00 | 0.00 | C |
| ATOM | 5964 | O | LEU | A 760 | 29.184 | 85.281 | −21.072 | 1.00 | 0.00 | O |
| ATOM | 5965 | CB | LEU | A 760 | 27.832 | 83.160 | −23.225 | 1.00 | 0.00 | C |
| ATOM | 5966 | CG | LEU | A 760 | 27.973 | 82.467 | −24.582 | 1.00 | 0.00 | C |
| ATOM | 5967 | CD1 | LEU | A 760 | 27.737 | 80.973 | −24.424 | 1.00 | 0.00 | C |
| ATOM | 5968 | CD2 | LEU | A 760 | 26.981 | 83.068 | −25.570 | 1.00 | 0.00 | C |
| ATOM | 5969 | N | PRO | A 761 | 26.942 | 85.365 | −21.200 | 1.00 | 0.00 | N |
| ATOM | 5970 | CA | PRO | A 761 | 26.864 | 85.818 | −19.808 | 1.00 | 0.00 | C |
| ATOM | 5971 | C | PRO | A 761 | 27.431 | 84.736 | −18.883 | 1.00 | 0.00 | C |
| ATOM | 5972 | O | PRO | A 761 | 26.947 | 83.601 | −18.881 | 1.00 | 0.00 | O |
| ATOM | 5973 | CB | PRO | A 761 | 25.366 | 86.030 | −19.600 | 1.00 | 0.00 | C |
| ATOM | 5974 | CG | PRO | A 761 | 24.892 | 86.421 | −20.970 | 1.00 | 0.00 | C |
| ATOM | 5975 | CD | PRO | A 761 | 25.626 | 85.446 | −21.857 | 1.00 | 0.00 | C |
| ATOM | 5976 | N | SER | A 762 | 28.466 | 85.093 | −18.126 | 1.00 | 0.00 | N |
| ATOM | 5977 | CA | SER | A 762 | 29.121 | 84.188 | −17.179 | 1.00 | 0.00 | C |
| ATOM | 5978 | C | SER | A 762 | 30.094 | 83.195 | −17.805 | 1.00 | 0.00 | C |
| ATOM | 5979 | O | SER | A 762 | 30.812 | 82.493 | −17.083 | 1.00 | 0.00 | O |
| ATOM | 5980 | CB | SER | A 762 | 28.082 | 83.391 | −16.378 | 1.00 | 0.00 | C |
| ATOM | 5981 | OG | SER | A 762 | 27.233 | 84.232 | −15.625 | 1.00 | 0.00 | O |
| ATOM | 5982 | N | VAL | A 763 | 30.132 | 83.124 | −19.131 | 1.00 | 0.00 | N |
| ATOM | 5983 | CA | VAL | A 763 | 31.010 | 82.157 | −19.778 | 1.00 | 0.00 | C |
| ATOM | 5984 | C | VAL | A 763 | 31.559 | 82.557 | −21.138 | 1.00 | 0.00 | C |
| ATOM | 5985 | O | VAL | A 763 | 30.807 | 82.948 | −22.029 | 1.00 | 0.00 | O |
| ATOM | 5986 | CB | VAL | A 763 | 30.280 | 80.800 | −19.988 | 1.00 | 0.00 | C |
| ATOM | 5987 | CG1 | VAL | A 763 | 31.230 | 79.784 | −20.609 | 1.00 | 0.00 | C |
| ATOM | 5988 | CG2 | VAL | A 763 | 29.729 | 80.280 | −18.669 | 1.00 | 0.00 | C |
| ATOM | 5989 | N | VAL | A 764 | 32.876 | 82.476 | −21.287 | 1.00 | 0.00 | N |
| ATOM | 5990 | CA | VAL | A 764 | 33.487 | 82.725 | −22.581 | 1.00 | 0.00 | C |
| ATOM | 5991 | C | VAL | A 764 | 33.659 | 81.279 | −23.043 | 1.00 | 0.00 | C |
| ATOM | 5992 | O | VAL | A 764 | 34.502 | 80.546 | −22.520 | 1.00 | 0.00 | O |
| ATOM | 5993 | CB | VAL | A 764 | 34.864 | 83.418 | −22.469 | 1.00 | 0.00 | C |
| ATOM | 5994 | CG1 | VAL | A 764 | 35.521 | 83.497 | −23.845 | 1.00 | 0.00 | C |
| ATOM | 5995 | CG2 | VAL | A 764 | 34.693 | 84.817 | −21.895 | 1.00 | 0.00 | C |
| ATOM | 5996 | N | HIS | A 765 | 32.813 | 80.875 | −23.986 | 1.00 | 0.00 | N |
| ATOM | 5997 | CA | HIS | A 765 | 32.786 | 79.517 | −24.528 | 1.00 | 0.00 | C |
| ATOM | 5998 | C | HIS | A 765 | 33.685 | 79.425 | −25.753 | 1.00 | 0.00 | C |
| ATOM | 5999 | O | HIS | A 765 | 33.576 | 80.239 | −26.670 | 1.00 | 0.00 | O |
| ATOM | 6000 | CB | HIS | A 765 | 31.339 | 79.163 | −24.900 | 1.00 | 0.00 | C |
| ATOM | 6001 | CG | HIS | A 765 | 31.153 | 77.752 | −25.364 | 1.00 | 0.00 | C |
| ATOM | 6002 | ND1 | HIS | A 765 | 30.622 | 77.439 | −26.597 | 1.00 | 0.00 | N |
| ATOM | 6003 | CD2 | HIS | A 765 | 31.413 | 76.572 | −24.755 | 1.00 | 0.00 | C |
| ATOM | 6004 | CE1 | HIS | A 765 | 30.564 | 76.126 | −26.728 | 1.00 | 0.00 | C |
| ATOM | 6005 | NE2 | HIS | A 765 | 31.039 | 75.577 | −25.624 | 1.00 | 0.00 | N |
| ATOM | 6006 | N | GLN | A 766 | 34.562 | 78.427 | −25.778 | 1.00 | 0.00 | N |
| ATOM | 6007 | CA | GLN | A 766 | 35.487 | 78.275 | −26.892 | 1.00 | 0.00 | C |
| ATOM | 6008 | C | GLN | A 766 | 35.557 | 76.852 | −27.433 | 1.00 | 0.00 | C |
| ATOM | 6009 | O | GLN | A 766 | 35.688 | 75.896 | −26.667 | 1.00 | 0.00 | O |
| ATOM | 6010 | CB | GLN | A 766 | 36.893 | 78.697 | −26.460 | 1.00 | 0.00 | C |
| ATOM | 6011 | CG | GLN | A 766 | 36.944 | 79.974 | −25.632 | 1.00 | 0.00 | C |
| ATOM | 6012 | CD | GLN | A 766 | 38.134 | 79.995 | −24.693 | 1.00 | 0.00 | C |
| ATOM | 6013 | OE1 | GLN | A 766 | 39.280 | 79.826 | −25.105 | 1.00 | 0.00 | O |
| ATOM | 6014 | NE2 | GLN | A 766 | 37.864 | 80.201 | −23.390 | 1.00 | 0.00 | N |
| ATOM | 6015 | N | THR | A 767 | 35.468 | 76.723 | −28.754 | 1.00 | 0.00 | N |
| ATOM | 6016 | CA | THR | A 767 | 35.569 | 75.425 | −29.408 | 1.00 | 0.00 | C |
| ATOM | 6017 | C | THR | A 767 | 36.832 | 75.486 | −30.262 | 1.00 | 0.00 | C |
| ATOM | 6018 | O | THR | A 767 | 36.919 | 76.272 | −31.209 | 1.00 | 0.00 | O |
| ATOM | 6019 | CB | THR | A 767 | 34.344 | 75.130 | −30.299 | 1.00 | 0.00 | C |
| ATOM | 6020 | OG1 | THR | A 767 | 33.153 | 75.219 | −29.510 | 1.00 | 0.00 | O |
| ATOM | 6021 | CG2 | THR | A 767 | 34.439 | 73.722 | −30.884 | 1.00 | 0.00 | C |
| ATOM | 6022 | N | ILE | A 768 | 37.815 | 74.663 | −29.912 | 1.00 | 0.00 | N |
| ATOM | 6023 | CA | ILE | A 768 | 39.093 | 74.647 | −30.614 | 1.00 | 0.00 | C |
| ATOM | 6024 | C | ILE | A 768 | 39.275 | 73.386 | −31.449 | 1.00 | 0.00 | C |
| ATOM | 6025 | O | ILE | A 768 | 39.007 | 72.275 | −30.984 | 1.00 | 0.00 | O |
| ATOM | 6026 | CB | ILE | A 768 | 40.252 | 74.759 | −29.605 | 1.00 | 0.00 | C |
| ATOM | 6027 | CG1 | ILE | A 768 | 39.978 | 75.917 | −28.645 | 1.00 | 0.00 | C |
| ATOM | 6028 | CG2 | ILE | A 768 | 41.566 | 74.975 | −30.337 | 1.00 | 0.00 | C |
| ATOM | 6029 | CD1 | ILE | A 768 | 40.959 | 76.008 | −27.490 | 1.00 | 0.00 | C |
| ATOM | 6030 | N | MET | A 769 | 39.736 | 73.564 | −32.683 | 1.00 | 0.00 | N |
| ATOM | 6031 | CA | MET | A 769 | 39.936 | 72.442 | −33.597 | 1.00 | 0.00 | C |
| ATOM | 6032 | C | MET | A 769 | 41.382 | 72.313 | −34.052 | 1.00 | 0.00 | C |
| ATOM | 6033 | O | MET | A 769 | 42.002 | 73.294 | −34.475 | 1.00 | 0.00 | O |
| ATOM | 6034 | CB | MET | A 769 | 39.028 | 72.607 | −34.817 | 1.00 | 0.00 | C |
| ATOM | 6035 | CG | MET | A 769 | 37.546 | 72.602 | −34.478 | 1.00 | 0.00 | C |
| ATOM | 6036 | SD | MET | A 769 | 36.523 | 73.314 | −35.787 | 1.00 | 0.00 | S |
| ATOM | 6037 | CE | MET | A 769 | 36.440 | 74.995 | −35.226 | 1.00 | 0.00 | C |
| ATOM | 6038 | N | ARG | A 770 | 41.913 | 71.095 | −33.976 | 1.00 | 0.00 | N |
| ATOM | 6039 | CA | ARG | A 770 | 43.288 | 70.841 | −34.377 | 1.00 | 0.00 | C |
| ATOM | 6040 | C | ARG | A 770 | 43.419 | 69.640 | −35.309 | 1.00 | 0.00 | C |
| ATOM | 6041 | O | ARG | A 770 | 44.521 | 69.142 | −35.537 | 1.00 | 0.00 | O |
| ATOM | 6042 | CB | ARG | A 770 | 44.157 | 70.639 | −33.135 | 1.00 | 0.00 | C |
| ATOM | 6043 | CG | ARG | A 770 | 44.138 | 71.835 | −32.200 | 1.00 | 0.00 | C |
| ATOM | 6044 | CD | ARG | A 770 | 44.962 | 71.604 | −30.946 | 1.00 | 0.00 | C |
| ATOM | 6045 | NE | ARG | A 770 | 44.858 | 72.743 | −30.040 | 1.00 | 0.00 | N |
| ATOM | 6046 | CZ | ARG | A 770 | 45.423 | 72.803 | −28.838 | 1.00 | 0.00 | C |
| ATOM | 6047 | NH1 | ARG | A 770 | 46.140 | 71.783 | −28.385 | 1.00 | 0.00 | N |
| ATOM | 6048 | NH2 | ARG | A 770 | 45.269 | 73.887 | −28.089 | 1.00 | 0.00 | N |
| ATOM | 6049 | N | GLY | A 771 | 42.295 | 69.176 | −35.842 | 1.00 | 0.00 | N |
| ATOM | 6050 | CA | GLY | A 771 | 42.335 | 68.047 | −36.754 | 1.00 | 0.00 | C |
| ATOM | 6051 | C | GLY | A 771 | 41.602 | 66.809 | −36.278 | 1.00 | 0.00 | C |
| ATOM | 6052 | O | GLY | A 771 | 41.377 | 65.880 | −37.057 | 1.00 | 0.00 | O |
| ATOM | 6053 | N | GLY | A 772 | 41.243 | 66.779 | −35.000 | 1.00 | 0.00 | N |
| ATOM | 6054 | CA | GLY | A 772 | 40.528 | 65.633 | −34.469 | 1.00 | 0.00 | C |
| ATOM | 6055 | C | GLY | A 772 | 39.339 | 66.091 | −33.653 | 1.00 | 0.00 | C |
| ATOM | 6056 | O | GLY | A 772 | 38.727 | 67.118 | −33.963 | 1.00 | 0.00 | O |
| ATOM | 6057 | N | ALA | A 773 | 38.999 | 65.337 | −32.613 | 1.00 | 0.00 | N |
| ATOM | 6058 | CA | ALA | A 773 | 37.885 | 65.720 | −31.765 | 1.00 | 0.00 | C |
| ATOM | 6059 | C | ALA | A 773 | 38.207 | 67.119 | −31.245 | 1.00 | 0.00 | C |
| ATOM | 6060 | O | ALA | A 773 | 39.340 | 67.399 | −30.848 | 1.00 | 0.00 | O |
| ATOM | 6061 | CB | ALA | A 773 | 37.740 | 64.742 | −30.603 | 1.00 | 0.00 | C |
| ATOM | 6062 | N | PRO | A 774 | 37.217 | 68.021 | −31.256 | 1.00 | 0.00 | N |
| ATOM | 6063 | CA | PRO | A 774 | 37.457 | 69.383 | −30.774 | 1.00 | 0.00 | C |
| ATOM | 6064 | C | PRO | A 774 | 37.691 | 69.460 | −29.270 | 1.00 | 0.00 | C |
| ATOM | 6065 | O | PRO | A 774 | 37.290 | 68.571 | −28.517 | 1.00 | 0.00 | O |
| ATOM | 6066 | CB | PRO | A 774 | 36189 | 70.122 | −31.192 | 1.00 | 0.00 | C |
| ATOM | 6067 | CG | PRO | A 774 | 35.139 | 69.055 | −31.102 | 1.00 | 0.00 | C |
| ATOM | 6068 | CD | PRO | A 774 | 35.832 | 67.863 | −31.734 | 1.00 | 0.00 | C |
| ATOM | 6069 | N | GLU | A 775 | 38.362 | 70.526 | −28.847 | 1.00 | 0.00 | N |
| ATOM | 6070 | CA | GLU | A 775 | 38.611 | 70.764 | −27.436 | 1.00 | 0.00 | C |
| ATOM | 6071 | C | GLU | A 775 | 37.663 | 71.897 | −27.075 | 1.00 | 0.00 | C |
| ATOM | 6072 | O | GLU | A 775 | 37.465 | 72.822 | −27.864 | 1.00 | 0.00 | O |
| ATOM | 6073 | CB | GLU | A 775 | 40.057 | 71.211 | −27.191 | 1.00 | 0.00 | C |
| ATOM | 6074 | CG | GLU | A 775 | 40.368 | 71.487 | −25.716 | 1.00 | 0.00 | C |
| ATOM | 6075 | CD | GLU | A 775 | 41.784 | 71.987 | −25.481 | 1.00 | 0.00 | C |
| ATOM | 6076 | OE1 | GLU | A 775 | 42.687 | 71.612 | −26.259 | 1.00 | 0.00 | O |
| ATOM | 6077 | OE2 | GLU | A 775 | 41.998 | 72.741 | −24.505 | 1.00 | 0.00 | O |
| ATOM | 6078 | N | ILE | A 776 | 37.054 | 71.814 | −25.902 | 1.00 | 0.00 | N |
| ATOM | 6079 | CA | ILE | A 776 | 36.141 | 72.857 | −25.465 | 1.00 | 0.00 | C |
| ATOM | 6080 | C | ILE | A 776 | 36.720 | 73.487 | −24.207 | 1.00 | 0.00 | C |
| ATOM | 6081 | O | ILE | A 776 | 37.174 | 72.775 | −23.308 | 1.00 | 0.00 | O |
| ATOM | 6082 | CB | ILE | A 776 | 34.749 | 72.292 | −25.110 | 1.00 | 0.00 | C |
| ATOM | 6083 | CG1 | ILE | A 776 | 34.221 | 71.414 | −26.249 | 1.00 | 0.00 | C |
| ATOM | 6084 | CG2 | ILE | A 776 | 33.793 | 73.440 | −24.799 | 1.00 | 0.00 | C |
| ATOM | 6085 | CD1 | ILE | A 776 | 34.067 | 72.127 | −27.579 | 1.00 | 0.00 | C |
| ATOM | 6086 | N | ARG | A 777 | 36.720 | 74.816 | −24.153 | 1.00 | 0.00 | N |
| ATOM | 6087 | CA | ARG | A 777 | 37.214 | 75.529 | −22.981 | 1.00 | 0.00 | C |
| ATOM | 6088 | C | ARG | A 777 | 36.171 | 76.558 | −22.568 | 1.00 | 0.00 | C |
| ATOM | 6089 | O | ARG | A 777 | 35.670 | 77.317 | −23.405 | 1.00 | 0.00 | O |
| ATOM | 6090 | CB | ARG | A 777 | 38.536 | 76.244 | −23.281 | 1.00 | 0.00 | C |
| ATOM | 6091 | CG | ARG | A 777 | 39.685 | 75.323 | −23.653 | 1.00 | 0.00 | C |
| ATOM | 6092 | CD | ARG | A 777 | 40.989 | 76.103 | −23.806 | 1.00 | 0.00 | C |
| ATOM | 6093 | NE | ARG | A 777 | 42.059 | 75.271 | −24.350 | 1.00 | 0.00 | N |
| ATOM | 6094 | CZ | ARG | A 777 | 43.229 | 75.738 | −24.779 | 1.00 | 0.00 | C |
| ATOM | 6095 | NH1 | ARG | A 777 | 43.487 | 77.040 | −24.726 | 1.00 | 0.00 | N |
| ATOM | 6096 | NH2 | ARG | A 777 | 44.139 | 74.907 | −25.274 | 1.00 | 0.00 | N |
| ATOM | 6097 | N | ASN | A 778 | 35.830 | 76.561 | −21.284 | 1.00 | 0.00 | N |
| ATOM | 6098 | CA | ASN | A 778 | 34.865 | 77.508 | −20.745 | 1.00 | 0.00 | C |
| ATOM | 6099 | C | ASN | A 778 | 35.516 | 78.372 | −19.680 | 1.00 | 0.00 | C |
| ATOM | 6100 | O | ASN | A 778 | 35.985 | 77.855 | −18.663 | 1.00 | 0.00 | O |
| ATOM | 6101 | CB | ASN | A 778 | 33.673 | 76.796 | −20.088 | 1.00 | 0.00 | C |
| ATOM | 6102 | CG | ASN | A 778 | 32.742 | 76.138 | −21.089 | 1.00 | 0.00 | C |
| ATOM | 6103 | OD1 | ASN | A 778 | 32.721 | 76.497 | −22.267 | 1.00 | 0.00 | O |
| ATOM | 6104 | ND2 | ASN | A 778 | 31.946 | 75.179 | −20.615 | 1.00 | 0.00 | N |
| ATOM | 6105 | N | LEU | A 779 | 35.561 | 79.681 | −19.911 | 1.00 | 0.00 | N |
| ATOM | 6106 | CA | LEU | A 779 | 36.104 | 80.589 | −18.907 | 1.00 | 0.00 | C |
| ATOM | 6107 | C | LEU | A 779 | 34.859 | 80.990 | −18.123 | 1.00 | 0.00 | C |
| ATOM | 6108 | O | LEU | A 779 | 34.120 | 81.890 | −18.524 | 1.00 | 0.00 | O |
| ATOM | 6109 | CB | LEU | A 779 | 36.753 | 81.814 | −19.561 | 1.00 | 0.00 | C |
| ATOM | 6110 | CG | LEU | A 779 | 37.246 | 82.901 | −18.596 | 1.00 | 0.00 | C |
| ATOM | 6111 | CD1 | LEU | A 779 | 38.246 | 82.320 | −17.611 | 1.00 | 0.00 | C |
| ATOM | 6112 | CD2 | LEU | A 779 | 37.878 | 84.034 | −19.390 | 1.00 | 0.00 | C |
| ATOM | 6113 | N | VAL | A 780 | 34.632 | 80.300 | −17.011 | 1.00 | 0.00 | N |
| ATOM | 6114 | CA | VAL | A 780 | 33.454 | 80.512 | −16.182 | 1.00 | 0.00 | C |
| ATOM | 6115 | C | VAL | A 780 | 33.615 | 81.518 | −15.045 | 1.00 | 0.00 | C |
| ATOM | 6116 | O | VAL | A 780 | 34.436 | 81.337 | −14.152 | 1.00 | 0.00 | O |
| ATOM | 6117 | CB | VAL | A 780 | 32.974 | 79.164 | −15.584 | 1.00 | 0.00 | C |
| ATOM | 6118 | CG1 | VAL | A 780 | 31.659 | 79.353 | −14.835 | 1.00 | 0.00 | C |
| ATOM | 6119 | CG2 | VAL | A 780 | 32.809 | 78.139 | −16.692 | 1.00 | 0.00 | C |
| ATOM | 6120 | N | ASP | A 781 | 32.815 | 82.578 | −15.095 | 1.00 | 0.00 | N |
| ATOM | 6121 | CA | ASP | A 781 | 32.827 | 83.615 | −14.066 | 1.00 | 0.00 | C |
| ATOM | 6122 | C | ASP | A 781 | 31.375 | 83.907 | −13.735 | 1.00 | 0.00 | C |
| ATOM | 6123 | O | ASP | A 781 | 30.738 | 84.758 | −14.361 | 1.00 | 0.00 | O |
| ATOM | 6124 | CB | ASP | A 781 | 33.507 | 84.886 | −14.575 | 1.00 | 0.00 | C |
| ATOM | 6125 | CG | ASP | A 781 | 33.572 | 85.976 | −13.514 | 1.00 | 0.00 | C |
| ATOM | 6126 | OD1 | ASP | A 781 | 34.037 | 87.091 | −13.835 | 1.00 | 0.00 | O |
| ATOM | 6127 | OD2 | ASP | A 781 | 33.164 | 85.717 | −12.360 | 1.00 | 0.00 | O |
| ATOM | 6128 | N | ILE | A 782 | 30.856 | 83.181 | −12.755 | 1.00 | 0.00 | N |
| ATOM | 6129 | CA | ILE | A 782 | 29.472 | 83.321 | −12.339 | 1.00 | 0.00 | C |
| ATOM | 6130 | C | ILE | A 782 | 29.208 | 84.669 | −11.659 | 1.00 | 0.00 | C |
| ATOM | 6131 | O | ILE | A 792 | 28.075 | 84.984 | −11.308 | 1.00 | 0.00 | O |
| ATOM | 6132 | CB | ILE | A 782 | 29.083 | 82.146 | −11.411 | 1.00 | 0.00 | C |
| ATOM | 6133 | CG1 | ILE | A 782 | 27.563 | 82.008 | −11.347 | 1.00 | 0.00 | C |
| ATOM | 6134 | CG2 | ILE | A 782 | 29.677 | 82.356 | −10.018 | 1.00 | 0.00 | C |
| ATOM | 6135 | CD1 | ILE | A 782 | 27.107 | 80.678 | −10.769 | 1.00 | 0.00 | C |
| ATOM | 6136 | N | GLY | A 783 | 30.263 | 85.460 | −11.483 | 1.00 | 0.00 | N |
| ATOM | 6137 | CA | GLY | A 783 | 30.123 | 86.777 | −10.877 | 1.00 | 0.00 | C |
| ATOM | 6138 | C | GLY | A 783 | 29.285 | 86.839 | −9.613 | 1.00 | 0.00 | C |
| ATOM | 6139 | O | GLY | A 783 | 29.548 | 86.114 | −8.652 | 1.00 | 0.00 | O |
| ATOM | 6140 | N | SER | A 784 | 28.274 | 87.703 | −9.606 | 1.00 | 0.00 | N |
| ATOM | 6141 | CA | SER | A 784 | 27.417 | 87.844 | −8.431 | 1.00 | 0.00 | C |
| ATOM | 6142 | C | SER | A 784 | 26.009 | 87.272 | −8.614 | 1.00 | 0.00 | C |
| ATOM | 6143 | O | SER | A 784 | 25.078 | 87.659 | −7.904 | 1.00 | 0.00 | O |
| ATOM | 6144 | CB | SER | A 784 | 27.330 | 89.317 | −8.012 | 1.00 | 0.00 | C |
| ATOM | 6145 | OG | SER | A 784 | 26.717 | 90.108 | −9.016 | 1.00 | 0.00 | O |
| ATOM | 6146 | N | LEU | A 785 | 25.856 | 86.349 | −9.560 | 1.00 | 0.00 | N |
| ATOM | 6147 | CA | LEU | A 785 | 24.562 | 85.718 | −9.814 | 1.00 | 0.00 | C |
| ATOM | 6148 | C | LEU | A 785 | 24.236 | 84.743 | −8.685 | 1.00 | 0.00 | C |
| ATOM | 6149 | O | LEU | A 785 | 24.466 | 83.536 | −8.806 | 1.00 | 0.00 | O |
| ATOM | 6150 | CB | LEU | A 785 | 24.588 | 84.964 | −11.147 | 1.00 | 0.00 | C |
| ATOM | 6151 | CG | LEU | A 785 | 24.509 | 85.760 | −12.453 | 1.00 | 0.00 | C |
| ATOM | 6152 | CD1 | LEU | A 785 | 25.549 | 86.864 | −12.471 | 1.00 | 0.00 | C |
| ATOM | 6153 | CD2 | LEU | A 785 | 24.720 | 84.815 | −13.625 | 1.00 | 0.00 | C |
| ATOM | 6154 | N | ASP | A 786 | 23.691 | 85.267 | −7.593 | 1.00 | 0.00 | N |
| ATOM | 6155 | CA | ASP | A 786 | 23.352 | 84.439 | −6.442 | 1.00 | 0.00 | C |
| ATOM | 6156 | C | ASP | A 786 | 22.287 | 83.385 | −6.729 | 1.00 | 0.00 | C |
| ATOM | 6157 | O | ASP | A 786 | 21.405 | 83.577 | −7.569 | 1.00 | 0.00 | O |
| ATOM | 6158 | CB | ASP | A 786 | 22.885 | 85.314 | −5.276 | 1.00 | 0.00 | C |
| ATOM | 6159 | CG | ASP | A 786 | 23.874 | 86.407 | −4.937 | 1.00 | 0.00 | C |
| ATOM | 6160 | OD1 | ASP | A 786 | 25.072 | 86.098 | −4.756 | 1.00 | 0.00 | O |
| ATOM | 6161 | OD2 | ASP | A 786 | 23.449 | 87.578 | −4.852 | 1.00 | 0.00 | O |
| ATOM | 6162 | N | ASN | A 787 | 22.382 | 82.274 | −6.006 | 1.00 | 0.00 | N |
| ATOM | 6163 | CA | ASN | A 787 | 21.451 | 81.162 | −6.139 | 1.00 | 0.00 | C |
| ATOM | 6164 | C | ASN | A 787 | 21.260 | 80.754 | −7.588 | 1.00 | 0.00 | C |
| ATOM | 6165 | O | ASN | A 787 | 20.140 | 80.615 | −8.076 | 1.00 | 0.00 | O |
| ATOM | 6166 | CB | ASN | A 787 | 20.113 | 81.525 | −5.496 | 1.00 | 0.00 | C |
| ATOM | 6167 | CG | ASN | A 787 | 20.255 | 81.836 | −4.021 | 1.00 | 0.00 | C |
| ATOM | 6168 | OD1 | ASN | A 787 | 20.920 | 81.103 | −3.286 | 1.00 | 0.00 | O |
| ATOM | 6169 | ND2 | ASN | A 787 | 19.636 | 82.923 | −3.578 | 1.00 | 0.00 | N |
| ATOM | 6170 | N | THR | A 788 | 22.380 | 80.553 | −8.267 | 1.00 | 0.00 | N |
| ATOM | 6171 | CA | THR | A 788 | 22.365 | 80.153 | −9.661 | 1.00 | 0.00 | C |
| ATOM | 6172 | C | THR | A 788 | 23.397 | 79.056 | −9.891 | 1.00 | 0.00 | C |
| ATOM | 6173 | O | THR | A 788 | 24.492 | 79.091 | −9.328 | 1.00 | 0.00 | O |
| ATOM | 6174 | CB | THR | A 788 | 22.699 | 81.349 | −10.579 | 1.00 | 0.00 | C |
| ATOM | 6175 | OG1 | THR | A 788 | 21.741 | 82.396 | −10.368 | 1.00 | 0.00 | O |
| ATOM | 6176 | CG2 | THR | A 788 | 22.665 | 80.929 | −12.045 | 1.00 | 0.00 | C |
| ATOM | 6177 | N | GLU | A 789 | 23.027 | 78.072 | −10.699 | 1.00 | 0.00 | N |
| ATOM | 6178 | CA | GLU | A 789 | 23.934 | 76.988 | −11.048 | 1.00 | 0.00 | C |
| ATOM | 6179 | C | GLU | A 789 | 23.926 | 76.936 | −12.568 | 1.00 | 0.00 | C |
| ATOM | 6180 | O | GLU | A 789 | 22.882 | 76.735 | −13.189 | 1.00 | 0.00 | O |
| ATOM | 6181 | CB | GLU | A 789 | 23.466 | 75.661 | −10.435 | 1.00 | 0.00 | C |
| ATOM | 6182 | CG | GLU | A 789 | 23.437 | 75.702 | −8.908 | 1.00 | 0.00 | C |
| ATOM | 6183 | CD | GLU | A 789 | 23.458 | 74.330 | −8.257 | 1.00 | 0.00 | C |
| ATOM | 6184 | OE1 | GLU | A 789 | 24.228 | 73.457 | −8.724 | 1.00 | 0.00 | O |
| ATOM | 6185 | OE2 | GLU | A 789 | 22.720 | 74.135 | −7.264 | 1.00 | 0.00 | O |
| ATOM | 6186 | N | ILE | A 790 | 25.093 | 77.156 | −13.162 | 1.00 | 0.00 | N |
| ATOM | 6187 | CA | ILE | A 790 | 25.225 | 77.159 | −14.610 | 1.00 | 0.00 | C |
| ATOM | 6188 | C | ILE | A 790 | 25.573 | 75.773 | −15.134 | 1.00 | 0.00 | C |
| ATOM | 6189 | O | ILE | A 790 | 26.529 | 75.146 | −14.677 | 1.00 | 0.00 | O |
| ATOM | 6190 | CB | ILE | A 790 | 26.315 | 78.145 | −15.059 | 1.00 | 0.00 | C |
| ATOM | 6191 | CG1 | ILE | A 790 | 26.005 | 79.542 | −14.509 | 1.00 | 0.00 | C |
| ATOM | 6192 | CG2 | ILE | A 790 | 26.389 | 78.174 | −16.579 | 1.00 | 0.00 | C |
| ATOM | 6193 | CD1 | ILE | A 790 | 27.126 | 80.549 | −14.712 | 1.00 | 0.00 | C |
| ATOM | 6194 | N | VAL | A 791 | 24.794 | 75.301 | −16.099 | 1.00 | 0.00 | N |
| ATOM | 6195 | CA | VAL | A 791 | 25.030 | 73.988 | −16.672 | 1.00 | 0.00 | C |
| ATOM | 6196 | C | VAL | A 791 | 25.280 | 74.077 | −18.171 | 1.00 | 0.00 | C |
| ATOM | 6197 | O | VAL | A 791 | 24.712 | 74.930 | −18.859 | 1.00 | 0.00 | O |
| ATOM | 6198 | CB | VAL | A 791 | 23.820 | 73.048 | −16.423 | 1.00 | 0.00 | C |
| ATOM | 6199 | CG1 | VAL | A 791 | 22.588 | 73.572 | −17.159 | 1.00 | 0.00 | C |
| ATOM | 6200 | CG2 | VAL | A 791 | 24.152 | 71.631 | −16.875 | 1.00 | 0.00 | C |
| ATOM | 6201 | N | MET | A 792 | 26.161 | 73.216 | −18.665 | 1.00 | 0.00 | N |
| ATOM | 6202 | CA | MET | A 792 | 26.445 | 73.163 | −20.093 | 1.00 | 0.00 | C |
| ATOM | 6203 | C | MET | A 792 | 25.810 | 71.864 | −20.561 | 1.00 | 0.00 | C |
| ATOM | 6204 | O | MET | A 792 | 26.145 | 70.791 | −20.061 | 1.00 | 0.00 | O |
| ATOM | 6205 | CB | MET | A 792 | 27.947 | 73.136 | −20.367 | 1.00 | 0.00 | C |
| ATOM | 6206 | CG | MET | A 792 | 28.275 | 72.990 | −21.849 | 1.00 | 0.00 | C |
| ATOM | 6207 | SD | MET | A 792 | 30.047 | 73.069 | −22.215 | 1.00 | 0.00 | S |
| ATOM | 6208 | CE | MET | A 792 | 30.643 | 71.606 | −21.365 | 1.00 | 0.00 | C |
| ATOM | 6209 | N | ARG | A 793 | 24.891 | 71.963 | −21.515 | 1.00 | 0.00 | N |
| ATOM | 6210 | CA | ARG | A 793 | 24.189 | 70.787 | −22.017 | 1.00 | 0.00 | C |
| ATOM | 6211 | C | ARG | A 793 | 24.423 | 70.534 | −23.500 | 1.00 | 0.00 | C |
| ATOM | 6212 | O | ARG | A 793 | 24.630 | 71.464 | −24.284 | 1.00 | 0.00 | O |
| ATOM | 6213 | CB | ARG | A 793 | 22.681 | 70.941 | −21.755 | 1.00 | 0.00 | C |
| ATOM | 6214 | CG | ARG | A 793 | 21.806 | 69.771 | −22.230 | 1.00 | 0.00 | C |
| ATOM | 6215 | CD | ARG | A 793 | 20.324 | 70.041 | −21.934 | 1.00 | 0.00 | C |
| ATOM | 6216 | NE | ARG | A 793 | 20.062 | 70.143 | −20.499 | 1.00 | 0.00 | N |
| ATOM | 6217 | CZ | ARG | A 793 | 18.972 | 70.694 | −19.971 | 1.00 | 0.00 | C |
| ATOM | 6218 | NH1 | ARG | A 793 | 18.030 | 71.199 | −20.759 | 1.00 | 0.00 | N |
| ATOM | 6219 | NH2 | ARG | A 793 | 18.826 | 70.751 | −18.652 | 1.00 | 0.00 | N |
| ATOM | 6220 | N | LEU | A 794 | 24.399 | 69.257 | −23.870 | 1.00 | 0.00 | N |
| ATOM | 6221 | CA | LEU | A 794 | 24.554 | 68.837 | −25.256 | 1.00 | 0.00 | C |
| ATOM | 6222 | C | LEU | A 794 | 23.252 | 68.146 | −25.657 | 1.00 | 0.00 | C |
| ATOM | 6223 | O | LEU | A 794 | 22.764 | 67.267 | −24.940 | 1.00 | 0.00 | O |
| ATOM | 6224 | CB | LEU | A 794 | 25.730 | 67.861 | −25.398 | 1.00 | 0.00 | C |
| ATOM | 6225 | CG | LEU | A 794 | 27.137 | 68.468 | −25.469 | 1.00 | 0.00 | C |
| ATOM | 6226 | CD1 | LEU | A 794 | 28.178 | 67.453 | −25.012 | 1.00 | 0.00 | C |
| ATOM | 6227 | CD2 | LEU | A 794 | 27.413 | 68.922 | −26.894 | 1.00 | 0.00 | C |
| ATOM | 6228 | N | GLU | A 795 | 22.680 | 68.557 | −26.787 | 1.00 | 0.00 | N |
| ATOM | 6229 | CA | GLU | A 795 | 21.439 | 67.962 | −27.276 | 1.00 | 0.00 | C |
| ATOM | 6230 | C | GLU | A 795 | 21.711 | 67.242 | −28.599 | 1.00 | 0.00 | C |
| ATOM | 6231 | C | GLU | A 795 | 22.264 | 67.828 | −29.528 | 1.00 | 0.00 | O |
| ATOM | 6232 | CB | GLU | A 795 | 20.369 | 69.049 | −27.466 | 1.00 | 0.00 | C |
| ATOM | 6233 | CG | GLU | A 795 | 20.155 | 69.914 | −26.229 | 1.00 | 0.00 | C |
| ATOM | 6234 | CD | GLU | A 795 | 19.116 | 71.010 | −26.426 | 1.00 | 0.00 | C |
| ATOM | 6235 | OE1 | GLU | A 795 | 19.075 | 71.614 | −27.520 | 1.00 | 0.00 | O |
| ATOM | 6236 | OE2 | GLU | A 795 | 18.351 | 71.280 | −25.477 | 1.00 | 0.00 | O |
| ATOM | 6237 | N | THR | A 796 | 21.338 | 65.965 | −28.677 | 1.00 | 0.00 | N |
| ATOM | 6238 | CA | THR | A 796 | 21.554 | 65.182 | −29.888 | 1.00 | 0.00 | C |
| ATOM | 6239 | C | THR | A 796 | 20.334 | 64.330 | −30.225 | 1.00 | 0.00 | C |
| ATOM | 6240 | O | THR | A 796 | 19.333 | 64.350 | −29.514 | 1.00 | 0.00 | O |
| ATOM | 6241 | CB | THR | A 796 | 22.746 | 64.212 | −29.744 | 1.00 | 0.00 | C |
| ATOM | 6242 | OG1 | THR | A 796 | 22.350 | 63.095 | −28.933 | 1.00 | 0.00 | O |
| ATOM | 6243 | CG2 | THR | A 796 | 23.940 | 64.915 | −29.102 | 1.00 | 0.00 | C |
| ATOM | 6244 | N | HIS | A 797 | 20.444 | 63.577 | −31.316 | 1.00 | 0.00 | N |
| ATOM | 6245 | CA | HIS | A 797 | 19.377 | 62.691 | −31.771 | 1.00 | 0.00 | C |
| ATOM | 6246 | C | HIS | A 797 | 19.678 | 61.252 | −31.369 | 1.00 | 0.00 | C |
| ATOM | 6247 | O | HIS | A 797 | 18.953 | 60.332 | −31.743 | 1.00 | 0.00 | O |
| ATOM | 6248 | CB | HIS | A 797 | 19.233 | 62.773 | −33.293 | 1.00 | 0.00 | C |
| ATOM | 6249 | CG | HIS | A 797 | 18.600 | 64.043 | −33.771 | 1.00 | 0.00 | C |
| ATOM | 6250 | ND1 | HIS | A 797 | 18.581 | 64.411 | −35.100 | 1.00 | 0.00 | N |
| ATOM | 6251 | CD2 | HIS | A 797 | 17.951 | 65.022 | −33.099 | 1.00 | 0.00 | C |
| ATOM | 6252 | CE1 | HIS | A 797 | 17.948 | 65.564 | −35.224 | 1.00 | 0.00 | C |
| ATOM | 6253 | NE2 | HIS | A 797 | 17.556 | 65.956 | −34.025 | 1.00 | 0.00 | N |
| ATOM | 6254 | N | ILE | A 798 | 20.751 | 61.060 | −30.607 | 1.00 | 0.00 | N |
| ATOM | 6255 | CA | ILE | A 798 | 21.133 | 59.725 | −30.159 | 1.00 | 0.00 | C |
| ATOM | 6256 | C | ILE | A 798 | 19.995 | 59.105 | −29.354 | 1.00 | 0.00 | C |
| ATOM | 6257 | O | ILE | A 798 | 19.467 | 59.721 | −28.429 | 1.00 | 0.00 | O |
| ATOM | 6258 | CB | ILE | A 798 | 22.422 | 59.779 | −29.301 | 1.00 | 0.00 | C |
| ATOM | 6259 | CG1 | ILE | A 798 | 23.571 | 60.338 | −30.151 | 1.00 | 0.00 | C |
| ATOM | 6260 | CG2 | ILE | A 798 | 22.772 | 58.386 | −28.789 | 1.00 | 0.00 | C |
| ATOM | 6261 | CD1 | ILE | A 798 | 24.880 | 60.542 | −29.396 | 1.00 | 0.00 | C |
| ATOM | 6262 | N | ASP | A 799 | 19.613 | 57.886 | −29.726 | 1.00 | 0.00 | N |
| ATOM | 6263 | CA | ASP | A 799 | 18.525 | 57.178 | −29.062 | 1.00 | 0.00 | C |
| ATOM | 6264 | C | ASP | A 799 | 19.025 | 56.428 | −27.829 | 1.00 | 0.00 | C |
| ATOM | 6265 | O | ASP | A 799 | 18.975 | 55.199 | −27.773 | 1.00 | 0.00 | O |
| ATOM | 6266 | CB | ASP | A 799 | 17.881 | 56.188 | −30.037 | 1.00 | 0.00 | C |
| ATOM | 6267 | CG | ASP | A 799 | 16.554 | 55.662 | −29.537 | 1.00 | 0.00 | C |
| ATOM | 6268 | OD1 | ASP | A 799 | 16.082 | 54.637 | −30.066 | 1.00 | 0.00 | O |
| ATOM | 6269 | OD2 | ASP | A 799 | 15.979 | 56.279 | −28.618 | 1.00 | 0.00 | O |
| ATOM | 6270 | N | SER | A 800 | 19.495 | 57.177 | −26.838 | 1.00 | 0.00 | N |
| ATOM | 6271 | CA | SER | A 800 | 20.023 | 56.589 | −25.612 | 1.00 | 0.00 | C |
| ATOM | 6272 | C | SER | A 800 | 18.949 | 56.194 | −24.599 | 1.00 | 0.00 | C |
| ATOM | 6273 | O | SER | A 800 | 19.200 | 55.378 | −23.713 | 1.00 | 0.00 | O |
| ATOM | 6274 | CB | SER | A 800 | 21.010 | 57.563 | −24.960 | 1.00 | 0.00 | C |
| ATOM | 6275 | OG | SER | A 800 | 20.379 | 58.800 | −24.680 | 1.00 | 0.00 | O |
| ATOM | 6276 | N | GLY | A 801 | 17.758 | 56.775 | −24.721 | 1.00 | 0.00 | N |
| ATOM | 6277 | CA | GLY | A 801 | 16.685 | 56.448 | −23.798 | 1.00 | 0.00 | C |
| ATOM | 6278 | C | GLY | A 801 | 16.937 | 56.952 | −22.389 | 1.00 | 0.00 | C |
| ATOM | 6279 | O | GLY | A 801 | 17.035 | 58.157 | −22.166 | 1.00 | 0.00 | O |
| ATOM | 6280 | N | ASP | A 802 | 17.039 | 56.033 | −21.433 | 1.00 | 0.00 | N |
| ATOM | 6281 | CA | ASP | A 802 | 17.288 | 56.403 | −20.043 | 1.00 | 0.00 | C |
| ATOM | 6282 | C | ASP | A 802 | 18.671 | 55.948 | −19.575 | 1.00 | 0.00 | C |
| ATOM | 6283 | O | ASP | A 802 | 18.971 | 55.981 | −18.383 | 1.00 | 0.00 | O |
| ATOM | 6284 | CB | ASP | A 802 | 16.202 | 55.809 | −19.124 | 1.00 | 0.00 | C |
| ATOM | 6285 | CG | ASP | A 802 | 16.090 | 54.292 | −19.230 | 1.00 | 0.00 | C |
| ATOM | 6286 | OD1 | ASP | A 802 | 15.193 | 53.724 | −18.566 | 1.00 | 0.00 | O |
| ATOM | 6287 | OD2 | ASP | A 802 | 16.887 | 53.663 | −19.963 | 1.00 | 0.00 | O |
| ATOM | 6288 | N | ILE | A 803 | 19.511 | 55.541 | −20.522 | 1.00 | 0.00 | N |
| ATOM | 6289 | CA | ILE | A 803 | 20.858 | 55.068 | −20.210 | 1.00 | 0.00 | C |
| ATOM | 6290 | C | ILE | A 803 | 21.979 | 56.057 | −20.542 | 1.00 | 0.00 | C |
| ATOM | 6291 | O | ILE | A 803 | 21.925 | 56.774 | −21.539 | 1.00 | 0.00 | O |
| ATOM | 6292 | CB | ILE | A 803 | 21.162 | 53.750 | −20.968 | 1.00 | 0.00 | C |
| ATOM | 6293 | CG1 | ILE | A 803 | 20.142 | 52.675 | −20.580 | 1.00 | 0.00 | C |
| ATOM | 6294 | CG2 | ILE | A 803 | 22.583 | 53.278 | −20.665 | 1.00 | 0.00 | C |
| ATOM | 6295 | CD1 | ILE | A 803 | 20.164 | 52.288 | −19.111 | 1.00 | 0.00 | C |
| ATOM | 6296 | N | PHE | A 804 | 22.995 | 56.093 | −19.686 | 1.00 | 0.00 | N |
| ATOM | 6297 | CA | PHE | A 804 | 24.162 | 56.931 | −19.915 | 1.00 | 0.00 | C |
| ATOM | 6298 | C | PHE | A 804 | 25.302 | 56.379 | −19.074 | 1.00 | 0.00 | C |
| ATOM | 6299 | O | PHE | A 804 | 25.082 | 55.549 | −18.196 | 1.00 | 0.00 | O |
| ATOM | 6300 | CB | PHE | A 804 | 23.879 | 58.421 | −19.615 | 1.00 | 0.00 | C |
| ATOM | 6301 | CG | PHE | A 804 | 23.519 | 58.728 | −18.185 | 1.00 | 0.00 | C |
| ATOM | 6302 | CD1 | PHE | A 804 | 24.453 | 59.317 | −17.330 | 1.00 | 0.00 | C |
| ATOM | 6303 | CD2 | PHE | A 804 | 22.229 | 58.500 | −17.711 | 1.00 | 0.00 | C |
| ATOM | 6304 | CE1 | PHE | A 804 | 24.103 | 59.682 | −16.027 | 1.00 | 0.00 | C |
| ATOM | 6305 | CE2 | PHE | A 804 | 21.867 | 58.857 | −16.411 | 1.00 | 0.00 | C |
| ATOM | 6306 | CZ | PHE | A 804 | 22.808 | 59.452 | −15.567 | 1.00 | 0.00 | C |
| ATOM | 6307 | N | TYR | A 805 | 26.522 | 56.805 | −19.367 | 1.00 | 0.00 | N |
| ATOM | 6308 | CA | TYR | A 805 | 27.673 | 56.305 | −18.632 | 1.00 | 0.00 | C |
| ATOM | 6309 | C | TYR | A 805 | 28.508 | 57.429 | −18.048 | 1.00 | 0.00 | C |
| ATOM | 6310 | O | TYR | A 805 | 28.648 | 58.492 | −18.654 | 1.00 | 0.00 | O |
| ATOM | 6311 | CB | TYR | A 805 | 28.549 | 55.444 | −19.548 | 1.00 | 0.00 | C |
| ATOM | 6312 | CG | TYR | A 805 | 27.851 | 54.218 | −20.108 | 1.00 | 0.00 | C |
| ATOM | 6313 | CD1 | TYR | A 805 | 26.859 | 54.335 | −21.086 | 1.00 | 0.00 | C |
| ATOM | 6314 | CD2 | TYR | A 805 | 28.187 | 52.939 | −19.663 | 1.00 | 0.00 | C |
| ATOM | 6315 | CE1 | TYR | A 805 | 26.223 | 53.205 | −21.610 | 1.00 | 0.00 | C |
| ATOM | 6316 | CE2 | TYR | A 805 | 27.555 | 51.802 | −20.179 | 1.00 | 0.00 | C |
| ATOM | 6317 | CZ | TYR | A 805 | 26.578 | 51.943 | −21.150 | 1.00 | 0.00 | C |
| ATOM | 6318 | OH | TYR | A 805 | 25.964 | 50.822 | −21.667 | 1.00 | 0.00 | O |
| ATOM | 6319 | N | THR | A 806 | 29.046 | 57.185 | −16.858 | 1.00 | 0.00 | N |
| ATOM | 6320 | CA | THR | A 806 | 29.902 | 58.148 | −16.182 | 1.00 | 0.00 | C |
| ATOM | 6321 | C | THR | A 806 | 31.062 | 57.341 | −15.608 | 1.00 | 0.00 | C |
| ATOM | 6322 | O | THR | A 806 | 30.941 | 56.130 | −15.415 | 1.00 | 0.00 | O |
| ATOM | 6323 | CB | THR | A 806 | 29.144 | 58.880 | −15.057 | 1.00 | 0.00 | C |
| ATOM | 6324 | OG1 | THR | A 806 | 28.760 | 57.945 | −14.038 | 1.00 | 0.00 | O |
| ATOM | 6325 | CG2 | THR | A 806 | 27.890 | 59.547 | −15.620 | 1.00 | 0.00 | C |
| ATOM | 6326 | N | ASP | A 807 | 32.192 | 57.989 | −15.352 | 1.00 | 0.00 | N |
| ATOM | 6327 | CA | ASP | A 807 | 33.331 | 57.260 | −14.823 | 1.00 | 0.00 | C |
| ATOM | 6328 | C | ASP | A 807 | 33.445 | 57.383 | −13.315 | 1.00 | 0.00 | C |
| ATOM | 6329 | O | ASP | A 807 | 32.869 | 58.282 | −12.702 | 1.00 | 0.00 | O |
| ATOM | 6330 | CB | ASP | A 807 | 34.633 | 57.738 | −15.473 | 1.00 | 0.00 | C |
| ATOM | 6331 | CG | ASP | A 807 | 35.089 | 59.090 | −14.958 | 1.00 | 0.00 | C |
| ATOM | 6332 | OD1 | ASP | A 807 | 36.236 | 59.180 | −14.478 | 1.00 | 0.00 | O |
| ATOM | 6333 | OD2 | ASP | A 807 | 34.313 | 60.060 | −15.038 | 1.00 | 0.00 | O |
| ATOM | 6334 | N | LEU | A 808 | 34.181 | 56.449 | −12.729 | 1.00 | 0.00 | N |
| ATOM | 6335 | CA | LEU | A 808 | 34.417 | 56.447 | −11.299 | 1.00 | 0.00 | C |
| ATOM | 6336 | C | LEU | A 808 | 35.910 | 56.649 | −11.086 | 1.00 | 0.00 | C |
| ATOM | 6337 | O | LEU | A 808 | 36.722 | 55.799 | −11.461 | 1.00 | 0.00 | O |
| ATOM | 6338 | CB | LEU | A 808 | 33.960 | 55.122 | −10.674 | 1.00 | 0.00 | C |
| ATOM | 6339 | CG | LEU | A 808 | 32.442 | 54.931 | −10.518 | 1.00 | 0.00 | C |
| ATOM | 6340 | CD1 | LEU | A 808 | 32.120 | 53.463 | −10.264 | 1.00 | 0.00 | C |
| ATOM | 6341 | CD2 | LEU | A 808 | 31.928 | 55.788 | −9.372 | 1.00 | 0.00 | C |
| ATOM | 6342 | N | ASN | A 809 | 36.259 | 57.804 | −10.527 | 1.00 | 0.00 | N |
| ATOM | 6343 | CA | ASN | A 809 | 37.643 | 58.149 | −10.217 | 1.00 | 0.00 | C |
| ATOM | 6344 | C | ASN | A 809 | 38.620 | 58.045 | −11.382 | 1.00 | 0.00 | C |
| ATOM | 6345 | O | ASN | A 809 | 39.803 | 57.783 | −11.170 | 1.00 | 0.00 | O |
| ATOM | 6346 | CB | ASN | A 809 | 38.122 | 57.266 | −9.069 | 1.00 | 0.00 | C |
| ATOM | 6347 | CG | ASN | A 809 | 37.132 | 57.230 | −7.927 | 1.00 | 0.00 | C |
| ATOM | 6348 | OD1 | ASN | A 809 | 37.117 | 58.117 | −7.065 | 1.00 | 0.00 | O |
| ATOM | 6349 | ND2 | ASN | A 809 | 36.274 | 56.220 | −7.929 | 1.00 | 0.00 | N |
| ATOM | 6350 | N | GLY | A 810 | 38.134 | 58.262 | −12.601 | 1.00 | 0.00 | N |
| ATOM | 6351 | CA | GLY | A 810 | 38.999 | 58.182 | −13.770 | 1.00 | 0.00 | C |
| ATOM | 6352 | C | GLY | A 810 | 39.588 | 56.797 | −13.976 | 1.00 | 0.00 | C |
| ATOM | 6353 | O | GLY | A 810 | 40.618 | 56.633 | −14.645 | 1.00 | 0.00 | O |
| ATOM | 6354 | N | LEU | A 811 | 38.920 | 55.795 | −13.418 | 1.00 | 0.00 | N |
| ATOM | 6355 | CA | LEU | A 811 | 39.381 | 54.415 | −13.499 | 1.00 | 0.00 | C |
| ATOM | 6356 | C | LEU | A 811 | 38.510 | 53.489 | −14.345 | 1.00 | 0.00 | C |
| ATOM | 6357 | O | LEU | A 811 | 39.022 | 52.647 | −15.085 | 1.00 | 0.00 | O |
| ATOM | 6358 | CB | LEU | A 811 | 39.485 | 53.835 | −12.084 | 1.00 | 0.00 | C |
| ATOM | 6359 | CG | LEU | A 811 | 39.808 | 52.343 | −11.975 | 1.00 | 0.00 | C |
| ATOM | 6360 | CD1 | LEU | A 811 | 41.209 | 52.096 | −12.523 | 1.00 | 0.00 | C |
| ATOM | 6361 | CD2 | LEU | A 811 | 39.700 | 51.887 | −10.524 | 1.00 | 0.00 | C |
| ATOM | 6362 | N | GLN | A 812 | 37.196 | 53.651 | −14.238 | 1.00 | 0.00 | N |
| ATOM | 6363 | CA | GLN | A 812 | 36.261 | 52.785 | −14.947 | 1.00 | 0.00 | C |
| ATOM | 6364 | C | GLN | A 812 | 34.993 | 53.540 | −15.291 | 1.00 | 0.00 | C |
| ATOM | 6365 | O | GLN | A 812 | 34.687 | 54.550 | −14.664 | 1.00 | 0.00 | O |
| ATOM | 6366 | CB | GLN | A 812 | 35.888 | 51.615 | −14.039 | 1.00 | 0.00 | C |
| ATOM | 6367 | CG | GLN | A 812 | 35.288 | 52.093 | −12.701 | 1.00 | 0.00 | C |
| ATOM | 6368 | CD | GLN | A 812 | 34.982 | 50.963 | −11.733 | 1.00 | 0.00 | C |
| ATOM | 6369 | OE1 | GLN | A 812 | 33.991 | 50.248 | −11.883 | 1.00 | 0.00 | O |
| ATOM | 6370 | NE2 | GLN | A 812 | 35.841 | 50.795 | −10.733 | 1.00 | 0.00 | N |
| ATOM | 6371 | N | PHE | A 813 | 34.258 | 53.045 | −16.284 | 1.00 | 0.00 | N |
| ATOM | 6372 | CA | PHE | A 813 | 32.996 | 53.658 | −16.667 | 1.00 | 0.00 | C |
| ATOM | 6373 | C | PHE | A 813 | 31.874 | 52.717 | −16.261 | 1.00 | 0.00 | C |
| ATOM | 6374 | O | PHE | A 813 | 31.888 | 51.516 | −16.576 | 1.00 | 0.00 | O |
| ATOM | 6375 | CB | PHE | A 813 | 32.953 | 53.956 | −18.170 | 1.00 | 0.00 | C |
| ATOM | 6376 | CG | PHE | A 813 | 33.703 | 55.199 | −18.548 | 1.00 | 0.00 | C |
| ATOM | 6377 | CD1 | PHE | A 813 | 35.093 | 55.204 | −18.581 | 1.00 | 0.00 | C |
| ATOM | 6378 | CD2 | PHE | A 813 | 33.020 | 56.388 | −18.801 | 1.00 | 0.00 | C |
| ATOM | 6379 | CE1 | PHE | A 813 | 35.798 | 56.374 | −18.856 | 1.00 | 0.00 | C |
| ATOM | 6380 | CE2 | PHE | A 813 | 33.718 | 57.563 | −19.077 | 1.00 | 0.00 | C |
| ATOM | 6381 | CZ | PHE | A 813 | 35.110 | 57.552 | −19.102 | 1.00 | 0.00 | C |
| ATOM | 6382 | N | ILE | A 814 | 30.912 | 53.269 | −15.536 | 1.00 | 0.00 | N |
| ATOM | 6383 | CA | ILE | A 814 | 29.798 | 52.490 | −15.034 | 1.00 | 0.00 | C |
| ATOM | 6384 | C | ILE | A 814 | 28.485 | 52.929 | −15.679 | 1.00 | 0.00 | C |
| ATOM | 6385 | O | ILE | A 814 | 28.260 | 54.114 | −15.924 | 1.00 | 0.00 | O |
| ATOM | 6386 | CB | ILE | A 814 | 29.721 | 52.621 | −13.491 | 1.00 | 0.00 | C |
| ATOM | 6387 | CG1 | ILE | A 814 | 28.672 | 51.656 | −12.926 | 1.00 | 0.00 | C |
| ATOM | 6388 | CG2 | ILE | A 814 | 29.409 | 54.063 | −13.101 | 1.00 | 0.00 | C |
| ATOM | 6389 | CD1 | ILE | A 814 | 28.665 | 51.582 | −11.396 | 1.00 | 0.00 | C |
| ATOM | 6390 | N | LYS | A 815 | 27.625 | 51.958 | −15.963 | 1.00 | 0.00 | N |
| ATOM | 6391 | CA | LYS | A 815 | 26.341 | 52.235 | −16.592 | 1.00 | 0.00 | C |
| ATOM | 6392 | C | LYS | A 815 | 25.380 | 52.879 | −15.600 | 1.00 | 0.00 | C |
| ATOM | 6393 | O | LYS | A 815 | 25.247 | 52.420 | −14.467 | 1.00 | 0.00 | O |
| ATOM | 6394 | CB | LYS | A 815 | 25.748 | 50.932 | −17.140 | 1.00 | 0.00 | C |
| ATOM | 6395 | CG | LYS | A 815 | 24.388 | 51.055 | −17.816 | 1.00 | 0.00 | C |
| ATOM | 6396 | CD | LYS | A 815 | 23.990 | 49.703 | −18.418 | 1.00 | 0.00 | C |
| ATOM | 6397 | CE | LYS | A 815 | 22.669 | 49.775 | −19.161 | 1.00 | 0.00 | C |
| ATOM | 6398 | NZ | LYS | A 815 | 22.303 | 48.456 | −19.762 | 1.00 | 0.00 | N |
| ATOM | 6399 | N | ARG | A 816 | 24.731 | 53.956 | −16.039 | 1.00 | 0.00 | N |
| ATOM | 6400 | CA | ARG | A 816 | 23.766 | 54.689 | −15.226 | 1.00 | 0.00 | C |
| ATOM | 6401 | C | ARG | A 816 | 22.400 | 54.588 | −15.881 | 1.00 | 0.00 | C |
| ATOM | 6402 | O | ARG | A 816 | 22.302 | 54.510 | −17.102 | 1.00 | 0.00 | O |
| ATOM | 6403 | CB | ARG | A 816 | 24.127 | 56.177 | −15.155 | 1.00 | 0.00 | C |
| ATOM | 6404 | CG | ARG | A 816 | 25.517 | 56.489 | −14.635 | 1.00 | 0.00 | C |
| ATOM | 6405 | CD | ARG | A 816 | 25.690 | 55.935 | −13.240 | 1.00 | 0.00 | C |
| ATOM | 6406 | NE | ARG | A 816 | 26.856 | 56.490 | −12.557 | 1.00 | 0.00 | N |
| ATOM | 6407 | CZ | ARG | A 816 | 27.229 | 56.123 | −11.337 | 1.00 | 0.00 | C |
| ATOM | 6408 | NH1 | ARG | A 816 | 26.527 | 55.204 | −10.688 | 1.00 | 0.00 | N |
| ATOM | 6409 | NH2 | ARG | A 816 | 28.287 | 56.679 | −10.763 | 1.00 | 0.00 | N |
| ATOM | 6410 | N | ARG | A 817 | 21.349 | 54.581 | −15.072 | 1.00 | 0.00 | N |
| ATOM | 6411 | CA | ARG | A 817 | 19.999 | 54.549 | −15.617 | 1.00 | 0.00 | C |
| ATOM | 6412 | C | ARG | A 817 | 19.210 | 55.684 | −14.982 | 1.00 | 0.00 | C |
| ATOM | 6413 | O | ARG | A 817 | 19.032 | 55.718 | −13.766 | 1.00 | 0.00 | O |
| ATOM | 6414 | CB | ARG | A 817 | 19.293 | 53.208 | −15.336 | 1.00 | 0.00 | C |
| ATOM | 6415 | CG | ARG | A 817 | 17.826 | 53.206 | −15.804 | 1.00 | 0.00 | C |
| ATOM | 6416 | CD | ARG | A 817 | 17.112 | 51.862 | −15.629 | 1.00 | 0.00 | C |
| ATOM | 6417 | NE | ARG | A 817 | 17.629 | 50.832 | −16.525 | 1.00 | 0.00 | N |
| ATOM | 6418 | CZ | ARG | A 817 | 18.366 | 49.799 | −16.133 | 1.00 | 0.00 | C |
| ATOM | 6419 | NH1 | ARG | A 817 | 18.677 | 49.650 | −14.852 | 1.00 | 0.00 | N |
| ATOM | 6420 | NH2 | ARG | A 817 | 18.794 | 48.912 | −17.022 | 1.00 | 0.00 | N |
| ATOM | 6421 | N | ARG | A 818 | 18.777 | 56.635 | −15.805 | 1.00 | 0.00 | N |
| ATOM | 6422 | CA | ARG | A 818 | 17.983 | 57.751 | −15.315 | 1.00 | 0.00 | C |
| ATOM | 6423 | C | ARG | A 818 | 16.706 | 57.142 | −14.748 | 1.00 | 0.00 | C |
| ATOM | 6424 | O | ARG | A 818 | 16.061 | 56.327 | −15.413 | 1.00 | 0.00 | O |
| ATOM | 6425 | CB | ARG | A 818 | 17.620 | 58.699 | −16.463 | 1.00 | 0.00 | C |
| ATOM | 6426 | CG | ARG | A 818 | 16.915 | 59.973 | −16.007 | 1.00 | 0.00 | C |
| ATOM | 6427 | CD | ARG | A 818 | 16.267 | 60.712 | −17.171 | 1.00 | 0.00 | C |
| ATOM | 6428 | NE | ARG | A 818 | 14.977 | 60.124 | −17.524 | 1.00 | 0.00 | N |
| ATOM | 6429 | CZ | ARG | A 818 | 14.699 | 59.550 | −18.693 | 1.00 | 0.00 | C |
| ATOM | 6430 | NH1 | ARG | A 818 | 15.620 | 59.477 | −19.647 | 1.00 | 0.00 | N |
| ATOM | 6431 | NH2 | ARG | A 818 | 13.492 | 59.042 | −18.904 | 1.00 | 0.00 | N |
| ATOM | 6432 | N | LEU | A 819 | 16.350 | 57.523 | −13.526 | 1.00 | 0.00 | N |
| ATOM | 6433 | CA | LEU | A 819 | 15.147 | 57.006 | −12.878 | 1.00 | 0.00 | C |
| ATOM | 6434 | C | LEU | A 819 | 14.178 | 58.149 | −12.604 | 1.00 | 0.00 | C |
| ATOM | 6435 | O | LEU | A 819 | 14.429 | 58.999 | −11.749 | 1.00 | 0.00 | O |
| ATOM | 6436 | CB | LEU | A 819 | 15.509 | 56.304 | −11.563 | 1.00 | 0.00 | C |
| ATOM | 6437 | CG | LEU | A 819 | 16.428 | 55.081 | −11.680 | 1.00 | 0.00 | C |
| ATOM | 6438 | CD1 | LEU | A 819 | 16.842 | 54.608 | −10.294 | 1.00 | 0.00 | C |
| ATOM | 6439 | CD2 | LEU | A 819 | 15.715 | 53.972 | −12.443 | 1.00 | 0.00 | C |
| ATOM | 6440 | N | ASP | A 820 | 13.066 | 58.169 | −13.328 | 1.00 | 0.00 | N |
| ATOM | 6441 | CA | ASP | A 820 | 12.095 | 59.230 | −13.140 | 1.00 | 0.00 | C |
| ATOM | 6442 | C | ASP | A 820 | 11.374 | 59.101 | −11.805 | 1.00 | 0.00 | C |
| ATOM | 6443 | O | ASP | A 820 | 10.713 | 60.039 | −11.359 | 1.00 | 0.00 | O |
| ATOM | 6444 | CB | ASP | A 820 | 11.104 | 59.254 | −14.307 | 1.00 | 0.00 | C |
| ATOM | 6445 | CG | ASP | A 820 | 11.786 | 59.536 | −15.638 | 1.00 | 0.00 | C |
| ATOM | 6446 | OD1 | ASP | A 820 | 12.746 | 60.335 | −15.654 | 1.00 | 0.00 | O |
| ATOM | 6447 | OD2 | ASP | A 820 | 11.365 | 58.970 | −16.669 | 1.00 | 0.00 | O |
| ATOM | 6448 | N | LYS | A 821 | 11.514 | 57.949 | −11.154 | 1.00 | 0.00 | N |
| ATOM | 6449 | CA | LYS | A 821 | 10.876 | 57.755 | −9.857 | 1.00 | 0.00 | C |
| ATOM | 6450 | C | LYS | A 821 | 11.670 | 58.516 | −8.795 | 1.00 | 0.00 | C |
| ATOM | 6451 | O | LYS | A 821 | 11.237 | 58.638 | −7.652 | 1.00 | 0.00 | O |
| ATOM | 6452 | CB | LYS | A 821 | 10.795 | 56.266 | −9.498 | 1.00 | 0.00 | C |
| ATOM | 6453 | CG | LYS | A 821 | 12.140 | 55.579 | −9.313 | 1.00 | 0.00 | C |
| ATOM | 6454 | CD | LYS | A 821 | 11.969 | 54.074 | −9.144 | 1.00 | 0.00 | C |
| ATOM | 6455 | CE | LYS | A 821 | 13.320 | 53.372 | −9.062 | 1.00 | 0.00 | C |
| ATOM | 6456 | NZ | LYS | A 821 | 13.179 | 51.886 | −9.108 | 1.00 | 0.00 | N |
| ATOM | 6457 | N | LEU | A 822 | 12.833 | 59.030 | −9.185 | 1.00 | 0.00 | N |
| ATOM | 6458 | CA | LEU | A 822 | 13.671 | 59.803 | −8.273 | 1.00 | 0.00 | C |
| ATOM | 6459 | C | LEU | A 822 | 13.718 | 61.258 | −8.737 | 1.00 | 0.00 | C |
| ATOM | 6460 | O | LEU | A 822 | 13.550 | 61.541 | −9.923 | 1.00 | 0.00 | O |
| ATOM | 6461 | CB | LEU | A 822 | 15.091 | 59.232 | −8.234 | 1.00 | 0.00 | C |
| ATOM | 6462 | CG | LEU | A 822 | 15.220 | 57.794 | −7.717 | 1.00 | 0.00 | C |
| ATOM | 6463 | CD1 | LEU | A 822 | 16.682 | 57.360 | −7.749 | 1.00 | 0.00 | C |
| ATOM | 6464 | CD2 | LEU | A 822 | 14.665 | 57.713 | −6.298 | 1.00 | 0.00 | C |
| ATOM | 6465 | N | PRO | A 823 | 13.941 | 62.201 | −7.807 | 1.00 | 0.00 | N |
| ATOM | 6466 | CA | PRO | A 823 | 14.000 | 63.615 | −8.192 | 1.00 | 0.00 | C |
| ATOM | 6467 | C | PRO | A 823 | 15.217 | 63.928 | −9.066 | 1.00 | 0.00 | C |
| ATOM | 6468 | O | PRO | A 823 | 16.190 | 63.175 | −9.090 | 1.00 | 0.00 | O |
| ATOM | 6469 | CB | PRO | A 823 | 14.024 | 64.340 | −6.848 | 1.00 | 0.00 | C |
| ATOM | 6470 | CG | PRO | A 823 | 14.727 | 63.362 | −5.949 | 1.00 | 0.00 | C |
| ATOM | 6471 | CD | PRO | A 823 | 14.111 | 62.037 | −6.353 | 1.00 | 0.00 | C |
| ATOM | 6472 | N | LEU | A 824 | 15.151 | 65.046 | −9.780 | 1.00 | 0.00 | N |
| ATOM | 6473 | CA | LEU | A 824 | 16.219 | 65.466 | −10.682 | 1.00 | 0.00 | C |
| ATOM | 6474 | C | LEU | A 824 | 17.632 | 65.338 | −10.100 | 1.00 | 0.00 | C |
| ATOM | 6475 | O | LEU | A 824 | 18.508 | 64.730 | −10.719 | 1.00 | 0.00 | O |
| ATOM | 6476 | CB | LEU | A 824 | 15.968 | 66.914 | −11.126 | 1.00 | 0.00 | C |
| ATOM | 6477 | CG | LEU | A 824 | 16.744 | 67.449 | −12.333 | 1.00 | 0.00 | C |
| ATOM | 6478 | CD1 | LEU | A 824 | 16.001 | 68.650 | −12.923 | 1.00 | 0.00 | C |
| ATOM | 6479 | CD2 | LEU | A 824 | 18.165 | 67.826 | −11.918 | 1.00 | 0.00 | C |
| ATOM | 6480 | N | GLN | A 825 | 17.844 | 65.902 | −8.913 | 1.00 | 0.00 | N |
| ATOM | 6481 | CA | GLN | A 825 | 19.159 | 65.874 | −8.271 | 1.00 | 0.00 | C |
| ATOM | 6482 | C | GLN | A 825 | 19.710 | 64.472 | −8.011 | 1.00 | 0.00 | C |
| ATOM | 6483 | O | GLN | A 825 | 20.928 | 64.285 | −7.907 | 1.00 | 0.00 | O |
| ATOM | 6484 | CB | GLN | A 825 | 19.123 | 66.651 | −6.951 | 1.00 | 0.00 | C |
| ATOM | 6485 | CG | GLN | A 825 | 18.152 | 66.095 | −5.918 | 1.00 | 0.00 | C |
| ATOM | 6486 | CD | GLN | A 825 | 16.753 | 66.679 | −6.042 | 1.00 | 0.00 | C |
| ATOM | 6487 | OE1 | GLN | A 825 | 16.340 | 67.104 | −7.118 | 1.00 | 0.00 | O |
| ATOM | 6488 | NE2 | GLN | A 825 | 16.013 | 66.685 | −4.939 | 1.00 | 0.00 | N |
| ATOM | 6489 | N | ALA | A 826 | 18.821 | 63.491 | −7.890 | 1.00 | 0.00 | N |
| ATOM | 6490 | CA | ALA | A 826 | 19.244 | 62.117 | −7.640 | 1.00 | 0.00 | C |
| ATOM | 6491 | C | ALA | A 826 | 19.845 | 61.505 | −8.902 | 1.00 | 0.00 | C |
| ATOM | 6492 | O | ALA | A 826 | 20.634 | 60.558 | −8.837 | 1.00 | 0.00 | O |
| ATOM | 6493 | CB | ALA | A 826 | 18.057 | 61.280 | −7.167 | 1.00 | 0.00 | C |
| ATOM | 6494 | N | ASN | A 827 | 19.468 | 62.048 | −10.057 | 1.00 | 0.00 | N |
| ATOM | 6495 | CA | ASN | A 827 | 19.975 | 61.541 | −11.322 | 1.00 | 0.00 | C |
| ATOM | 6496 | C | ASN | A 827 | 21.291 | 62.199 | −11.727 | 1.00 | 0.00 | C |
| ATOM | 6497 | O | ASN | A 827 | 21.815 | 61.952 | −12.811 | 1.00 | 0.00 | O |
| ATOM | 6498 | CB | ASN | A 827 | 18.910 | 61.696 | −12.412 | 1.00 | 0.00 | C |
| ATOM | 6499 | CG | ASN | A 827 | 17.765 | 60.710 | −12.236 | 1.00 | 0.00 | C |
| ATOM | 6500 | OD1 | ASN | A 827 | 17.960 | 59.498 | −12.330 | 1.00 | 0.00 | O |
| ATOM | 6501 | ND2 | ASN | A 827 | 16.569 | 61.224 | −11.966 | 1.00 | 0.00 | N |
| ATOM | 6502 | N | TYR | A 828 | 21.820 | 63.046 | −10.848 | 1.00 | 0.00 | N |
| ATOM | 6503 | CA | TYR | A 828 | 23.109 | 63.677 | −11.101 | 1.00 | 0.00 | C |
| ATOM | 6504 | C | TYR | A 828 | 24.151 | 62.746 | −10.487 | 1.00 | 0.00 | C |
| ATOM | 6505 | O | TYR | A 828 | 23.953 | 62.223 | −9.388 | 1.00 | 0.00 | O |
| ATOM | 6506 | CB | TYR | A 828 | 23.199 | 65.058 | −10.442 | 1.00 | 0.00 | C |
| ATOM | 6507 | CG | TYR | A 828 | 23.228 | 66.206 | −11.437 | 1.00 | 0.00 | C |
| ATOM | 6508 | CD1 | TYR | A 828 | 22.159 | 66.432 | −12.301 | 1.00 | 0.00 | C |
| ATOM | 6509 | CD2 | TYR | A 828 | 24.335 | 67.054 | −11.524 | 1.00 | 0.00 | C |
| ATOM | 6510 | CE1 | TYR | A 828 | 22.188 | 67.474 | −13.233 | 1.00 | 0.00 | C |
| ATOM | 6511 | CE2 | TYR | A 828 | 24.376 | 68.096 | −12.450 | 1.00 | 0.00 | C |
| ATOM | 6512 | CZ | TYR | A 828 | 23.303 | 68.300 | −13.301 | 1.00 | 0.00 | C |
| ATOM | 6513 | OH | TYR | A 828 | 23.352 | 69.314 | −14.230 | 1.00 | 0.00 | O |
| ATOM | 6514 | N | TYR | A 829 | 25.245 | 62.536 | −11.210 | 1.00 | 0.00 | N |
| ATOM | 6515 | CA | TYR | A 829 | 26.328 | 61.670 | −10.757 | 1.00 | 0.00 | C |
| ATOM | 6516 | C | TYR | A 829 | 27.674 | 62.365 | −10.911 | 1.00 | 0.00 | C |
| ATOM | 6517 | O | TYR | A 829 | 27.793 | 63.362 | −11.619 | 1.00 | 0.00 | O |
| ATOM | 6518 | CB | TYR | A 829 | 26.364 | 60.372 | −11.577 | 1.00 | 0.00 | C |
| ATOM | 6519 | CG | TYR | A 829 | 25.201 | 59.448 | −11.321 | 1.00 | 0.00 | C |
| ATOM | 6520 | CD1 | TYR | A 829 | 24.039 | 59.520 | −12.090 | 1.00 | 0.00 | C |
| ATOM | 6521 | CD2 | TYR | A 829 | 25.245 | 58.530 | −10.273 | 1.00 | 0.00 | C |
| ATOM | 6522 | CE1 | TYR | A 829 | 22.939 | 58.696 | −11.814 | 1.00 | 0.00 | C |
| ATOM | 6523 | CE2 | TYR | A 829 | 24.160 | 57.710 | −9.986 | 1.00 | 0.00 | C |
| ATOM | 6524 | CZ | TYR | A 829 | 23.011 | 57.797 | −10.755 | 1.00 | 0.00 | C |
| ATOM | 6525 | OH | TYR | A 829 | 21.935 | 56.997 | −10.440 | 1.00 | 0.00 | O |
| ATOM | 6526 | N | PRO | A 830 | 28.709 | 61.844 | −10.238 | 1.00 | 0.00 | N |
| ATOM | 6527 | CA | PRO | A 830 | 30.028 | 62.468 | −10.361 | 1.00 | 0.00 | C |
| ATOM | 6528 | C | PRO | A 830 | 30.532 | 62.285 | −11.793 | 1.00 | 0.00 | C |
| ATOM | 6529 | O | PRO | A 830 | 30.314 | 61.231 | −12.396 | 1.00 | 0.00 | O |
| ATOM | 6530 | CB | PRO | A 830 | 30.890 | 61.664 | −9.383 | 1.00 | 0.00 | C |
| ATOM | 6531 | CG | PRO | A 830 | 29.910 | 61.115 | −8.395 | 1.00 | 0.00 | C |
| ATOM | 6532 | CD | PRO | A 830 | 28.725 | 60.749 | −9.254 | 1.00 | 0.00 | C |
| ATOM | 6533 | N | ILE | A 831 | 31.180 | 63.307 | −12.346 | 1.00 | 0.00 | N |
| ATOM | 6534 | CA | ILE | A 831 | 31.764 | 63.192 | −13.679 | 1.00 | 0.00 | C |
| ATOM | 6535 | C | ILE | A 831 | 33.240 | 63.521 | −13.457 | 1.00 | 0.00 | C |
| ATOM | 6536 | O | ILE | A 831 | 33.726 | 64.574 | −13.872 | 1.00 | 0.00 | O |
| ATOM | 6537 | CB | ILE | A 831 | 31.182 | 64.205 | −14.703 | 1.00 | 0.00 | C |
| ATOM | 6538 | CG1 | ILE | A 831 | 29.648 | 64.168 | −14.705 | 1.00 | 0.00 | C |
| ATOM | 6539 | CG2 | ILE | A 831 | 31.711 | 63.868 | −16.093 | 1.00 | 0.00 | C |
| ATOM | 6540 | CD1 | ILE | A 831 | 29.037 | 62.791 | −15.017 | 1.00 | 0.00 | C |
| ATOM | 6541 | N | PRO | A 832 | 33.972 | 62.621 | −12.781 | 1.00 | 0.00 | N |
| ATOM | 6542 | CA | PRO | A 832 | 35.392 | 62.873 | −12.520 | 1.00 | 0.00 | C |
| ATOM | 6543 | C | PRO | A 832 | 36.283 | 63.046 | −13.739 | 1.00 | 0.00 | C |
| ATOM | 6544 | O | PRO | A 832 | 37.271 | 63.772 | −13.671 | 1.00 | 0.00 | O |
| ATOM | 6545 | CB | PRO | A 832 | 35.805 | 61.690 | −11.633 | 1.00 | 0.00 | C |
| ATOM | 6546 | CG | PRO | A 832 | 34.813 | 60.611 | −11.986 | 1.00 | 0.00 | C |
| ATOM | 6547 | CD | PRO | A 832 | 33.525 | 61.377 | −12.131 | 1.00 | 0.00 | C |
| ATOM | 6548 | N | SER | A 833 | 35.952 | 62.398 | −14.853 | 1.00 | 0.00 | N |
| ATOM | 6549 | CA | SER | A 833 | 36.780 | 62.552 | −16.046 | 1.00 | 0.00 | C |
| ATOM | 6550 | C | SER | A 833 | 36.050 | 62.332 | −17.369 | 1.00 | 0.00 | C |
| ATOM | 6551 | O | SER | A 833 | 36.580 | 62.673 | −18.424 | 1.00 | 0.00 | O |
| ATOM | 6552 | CB | SER | A 833 | 37.997 | 61.619 | −15.979 | 1.00 | 0.00 | C |
| ATOM | 6553 | OG | SER | A 833 | 37.652 | 60.284 | −16.291 | 1.00 | 0.00 | O |
| ATOM | 6554 | N | GLY | A 834 | 34.844 | 61.769 | −17.325 | 1.00 | 0.00 | N |
| ATOM | 6555 | CA | GLY | A 834 | 34.122 | 61.546 | −18.568 | 1.00 | 0.00 | C |
| ATOM | 6556 | C | GLY | A 834 | 32.723 | 60.967 | −18.453 | 1.00 | 0.00 | C |
| ATOM | 6557 | O | GLY | A 834 | 32.341 | 60.393 | −17.431 | 1.00 | 0.00 | O |
| ATOM | 6558 | N | MET | A 835 | 31.960 | 61.118 | −19.531 | 1.00 | 0.00 | N |
| ATOM | 6559 | CA | MET | A 835 | 30.591 | 60.625 | −19.589 | 1.00 | 0.00 | C |
| ATOM | 6560 | C | MET | A 835 | 30.216 | 60.423 | −21.057 | 1.00 | 0.00 | C |
| ATOM | 6561 | O | MET | A 835 | 30.786 | 61.067 | −21.948 | 1.00 | 0.00 | O |
| ATOM | 6562 | CB | MET | A 835 | 29.643 | 61.647 | −18.953 | 1.00 | 0.00 | C |
| ATOM | 6563 | CG | MET | A 835 | 29.514 | 62.949 | −19.743 | 1.00 | 0.00 | C |
| ATOM | 6564 | SD | MET | A 835 | 28.686 | 64.278 | −18.834 | 1.00 | 0.00 | S |
| ATOM | 6565 | CE | MET | A 835 | 27.089 | 63.534 | −18.505 | 1.00 | 0.00 | C |
| ATOM | 6566 | N | PHE | A 836 | 29.271 | 59.529 | −21.317 | 1.00 | 0.00 | N |
| ATOM | 6567 | CA | PHE | A 836 | 28.848 | 59.313 | −22.692 | 1.00 | 0.00 | C |
| ATOM | 6568 | C | PHE | A 836 | 27.483 | 58.672 | −22.818 | 1.00 | 0.00 | C |
| ATOM | 6569 | O | PHE | A 836 | 26.968 | 58.083 | −21.867 | 1.00 | 0.00 | O |
| ATOM | 6570 | CB | PHE | A 836 | 29.909 | 58.507 | −23.478 | 1.00 | 0.00 | C |
| ATOM | 6571 | CG | PHE | A 836 | 30.067 | 57.057 | −23.057 | 1.00 | 0.00 | C |
| ATOM | 6572 | CD1 | PHE | A 836 | 29.195 | 56.074 | −23.527 | 1.00 | 0.00 | C |
| ATOM | 6573 | CD2 | PHE | A 836 | 31.134 | 56.667 | −22.247 | 1.00 | 0.00 | C |
| ATOM | 6574 | CE1 | PHE | A 836 | 29.385 | 54.724 | −23.203 | 1.00 | 0.00 | C |
| ATOM | 6575 | CE2 | PHE | A 836 | 31.336 | 55.316 | −21.914 | 1.00 | 0.00 | C |
| ATOM | 6576 | CZ | PHE | A 836 | 30.459 | 54.344 | −22.395 | 1.00 | 0.00 | C |
| ATOM | 6577 | N | ILE | A 837 | 26.883 | 58.847 | −23.993 | 1.00 | 0.00 | N |
| ATOM | 6578 | CA | ILE | A 837 | 25.592 | 58.257 | −24.305 | 1.00 | 0.00 | C |
| ATOM | 6579 | C | ILE | A 837 | 25.758 | 57.597 | −25.661 | 1.00 | 0.00 | C |
| ATOM | 6580 | O | ILE | A 837 | 26.611 | 58.004 | −26.458 | 1.00 | 0.00 | O |
| ATOM | 6581 | CB | ILE | A 837 | 24.464 | 59.307 | −24.406 | 1.00 | 0.00 | C |
| ATOM | 6582 | CG1 | ILE | A 837 | 24.918 | 60.484 | −25.273 | 1.00 | 0.00 | C |
| ATOM | 6583 | CG2 | ILE | A 837 | 24.026 | 59.733 | −23.010 | 1.00 | 0.00 | C |
| ATOM | 6584 | CD1 | ILE | A 837 | 23.768 | 61.397 | −25.714 | 1.00 | 0.00 | C |
| ATOM | 6585 | N | GLU | A 838 | 24.955 | 56.577 | −25.930 | 1.00 | 0.00 | N |
| ATOM | 6586 | CA | GLU | A 838 | 25.063 | 55.886 | −27.205 | 1.00 | 0.00 | C |
| ATOM | 6587 | C | GLU | A 838 | 23.806 | 55.099 | −27.540 | 1.00 | 0.00 | C |
| ATOM | 6588 | O | GLU | A 838 | 22.977 | 54.828 | −26.673 | 1.00 | 0.00 | O |
| ATOM | 6589 | CB | GLU | A 838 | 26.233 | 54.898 | −27.155 | 1.00 | 0.00 | C |
| ATOM | 6590 | CG | GLU | A 838 | 25.979 | 53.750 | −26.165 | 1.00 | 0.00 | C |
| ATOM | 6591 | CD | GLU | A 838 | 27.094 | 52.717 | −26.117 | 1.00 | 0.00 | C |
| ATOM | 6592 | OE1 | GLU | A 838 | 26.943 | 51.721 | −25.377 | 1.00 | 0.00 | O |
| ATOM | 6593 | OE2 | GLU | A 838 | 28.116 | 52.887 | −26.810 | 1.00 | 0.00 | O |
| ATOM | 6594 | N | ASP | A 839 | 23.653 | 54.773 | −28.818 | 1.00 | 0.00 | N |
| ATOM | 6595 | CA | ASP | A 839 | 22.558 | 53.914 | −29.240 | 1.00 | 0.00 | C |
| ATOM | 6596 | C | ASP | A 839 | 23.263 | 52.824 | −30.031 | 1.00 | 0.00 | C |
| ATOM | 6597 | O | ASP | A 839 | 24.467 | 52.624 | −29.862 | 1.00 | 0.00 | O |
| ATOM | 6598 | CB | ASP | A 839 | 21.489 | 54.635 | −30.083 | 1.00 | 0.00 | C |
| ATOM | 6599 | CG | ASP | A 839 | 22.055 | 55.412 | −31.255 | 1.00 | 0.00 | C |
| ATOM | 6600 | OD1 | ASP | A 839 | 23.085 | 55.010 | −31.835 | 1.00 | 0.00 | O |
| ATOM | 6601 | OD2 | ASP | A 839 | 21.428 | 56.433 | −31.608 | 1.00 | 0.00 | O |
| ATOM | 6602 | N | ALA | A 840 | 22.542 | 52.116 | −30.887 | 1.00 | 0.00 | N |
| ATOM | 6603 | CA | ALA | A 840 | 23.160 | 51.045 | −31.649 | 1.00 | 0.00 | C |
| ATOM | 6604 | C | ALA | A 840 | 24.259 | 51.502 | −32.604 | 1.00 | 0.00 | C |
| ATOM | 6605 | O | ALA | A 840 | 25.201 | 50.751 | −32.873 | 1.00 | 0.00 | O |
| ATOM | 6606 | CB | ALA | A 840 | 22.085 | 50.281 | −32.425 | 1.00 | 0.00 | C |
| ATOM | 6607 | N | ASN | A 841 | 24.167 | 52.737 | −33.095 | 1.00 | 0.00 | N |
| ATOM | 6608 | CA | ASN | A 841 | 25.140 | 53.219 | −34.073 | 1.00 | 0.00 | C |
| ATOM | 6609 | C | ASN | A 841 | 26.023 | 54.406 | −33.721 | 1.00 | 0.00 | C |
| ATOM | 6610 | O | ASN | A 841 | 27.131 | 54.527 | −34.249 | 1.00 | 0.00 | O |
| ATOM | 6611 | CB | ASN | A 841 | 24.413 | 53.556 | −35.376 | 1.00 | 0.00 | C |
| ATOM | 6612 | CG | ASN | A 841 | 23.606 | 52.397 | −35.901 | 1.00 | 0.00 | C |
| ATOM | 6613 | OD1 | ASN | A 841 | 24.143 | 51.316 | −36.129 | 1.00 | 0.00 | O |
| ATOM | 6614 | ND2 | ASN | A 841 | 22.308 | 52.610 | −36.094 | 1.00 | 0.00 | N |
| ATOM | 6615 | N | THR | A 842 | 25.540 | 55.281 | −32.847 | 1.00 | 0.00 | N |
| ATOM | 6616 | CA | THR | A 842 | 26.290 | 56.485 | −32.513 | 1.00 | 0.00 | C |
| ATOM | 6617 | C | THR | A 842 | 26.538 | 56.707 | −31.027 | 1.00 | 0.00 | C |
| ATOM | 6618 | O | THR | A 842 | 25.701 | 56.384 | −30.187 | 1.00 | 0.00 | O |
| ATOM | 6619 | CB | THR | A 842 | 25.551 | 57.727 | −33.069 | 1.00 | 0.00 | C |
| ATOM | 6620 | OG1 | THR | A 842 | 25.196 | 57.494 | −34.440 | 1.00 | 0.00 | O |
| ATOM | 6621 | CG2 | THR | A 842 | 26.430 | 58.968 | −32.986 | 1.00 | 0.00 | C |
| ATOM | 6622 | N | ARG | A 843 | 27.698 | 57.272 | −30.714 | 1.00 | 0.00 | N |
| ATOM | 6623 | CA | ARG | A 843 | 28.052 | 57.575 | −29.331 | 1.00 | 0.00 | C |
| ATOM | 6624 | C | ARG | A 843 | 28.682 | 58.955 | −29.264 | 1.00 | 0.00 | C |
| ATOM | 6625 | O | ARG | A 843 | 29.399 | 59.368 | −30.175 | 1.00 | 0.00 | O |
| ATOM | 6626 | CB | ARG | A 843 | 29.050 | 56.557 | −28.768 | 1.00 | 0.00 | C |
| ATOM | 6627 | CG | ARG | A 843 | 29.576 | 56.934 | −27.372 | 1.00 | 0.00 | C |
| ATOM | 6628 | CD | ARG | A 843 | 30.763 | 56.082 | −26.918 | 1.00 | 0.00 | C |
| ATOM | 6629 | NE | ARG | A 843 | 30.391 | 54.703 | −26.602 | 1.00 | 0.00 | N |
| ATOM | 6630 | CZ | ARG | A 843 | 31.211 | 53.823 | −26.030 | 1.00 | 0.00 | C |
| ATOM | 6631 | NH1 | ARG | A 843 | 32.449 | 54.178 | −25.709 | 1.00 | 0.00 | N |
| ATOM | 6632 | NH2 | ARG | A 843 | 30.800 | 52.586 | −25.777 | 1.00 | 0.00 | N |
| ATOM | 6633 | N | LEU | A 844 | 28.397 | 59.667 | −28.182 | 1.00 | 0.00 | N |
| ATOM | 6634 | CA | LEU | A 844 | 28.971 | 60.986 | −27.965 | 1.00 | 0.00 | C |
| ATOM | 6635 | C | LEU | A 844 | 29.623 | 60.924 | −26.595 | 1.00 | 0.00 | C |
| ATOM | 6636 | O | LEU | A 844 | 28.956 | 60.630 | −25.598 | 1.00 | 0.00 | O |
| ATOM | 6637 | CB | LEU | A 844 | 27.894 | 62.074 | −27.968 | 1.00 | 0.00 | C |
| ATOM | 6638 | CG | LEU | A 844 | 28.464 | 63.490 | −27.833 | 1.00 | 0.00 | C |
| ATOM | 6639 | CD1 | LEU | A 844 | 29.378 | 63.776 | −29.019 | 1.00 | 0.00 | C |
| ATOM | 6640 | CD2 | LEU | A 844 | 27.338 | 64.513 | −27.767 | 1.00 | 0.00 | C |
| ATOM | 6641 | N | THR | A 845 | 30.925 | 61.187 | −26.548 | 1.00 | 0.00 | N |
| ATOM | 6642 | CA | THR | A 845 | 31.660 | 61.149 | −25.289 | 1.00 | 0.00 | C |
| ATOM | 6643 | C | THR | A 845 | 32.245 | 62.516 | −24.960 | 1.00 | 0.00 | C |
| ATOM | 6644 | O | THR | A 845 | 32.869 | 63.155 | −25.804 | 1.00 | 0.00 | O |
| ATOM | 6645 | CB | THR | A 845 | 32.827 | 60.140 | −25.351 | 1.00 | 0.00 | C |
| ATOM | 6646 | OG1 | THR | A 845 | 32.325 | 58.846 | −25.710 | 1.00 | 0.00 | O |
| ATOM | 6647 | CG2 | THR | A 845 | 33.535 | 60.051 | −23.999 | 1.00 | 0.00 | C |
| ATOM | 6648 | N | LEU | A 846 | 32.039 | 62.960 | −23.727 | 1.00 | 0.00 | N |
| ATOM | 6649 | CA | LEU | A 846 | 32.576 | 64.238 | −23.288 | 1.00 | 0.00 | C |
| ATOM | 6650 | C | LEU | A 846 | 33.588 | 63.923 | −22.194 | 1.00 | 0.00 | C |
| ATOM | 6651 | O | LEU | A 846 | 33.230 | 63.362 | −21.158 | 1.00 | 0.00 | O |
| ATOM | 6652 | CB | LEU | A 846 | 31.459 | 65.128 | −22.732 | 1.00 | 0.00 | C |
| ATOM | 6653 | CG | LEU | A 846 | 31.881 | 66.487 | −22.161 | 1.00 | 0.00 | C |
| ATOM | 6654 | CD1 | LEU | A 846 | 32.430 | 67.364 | −23.280 | 1.00 | 0.00 | C |
| ATOM | 6655 | CD2 | LEU | A 846 | 30.689 | 67.168 | −21.488 | 1.00 | 0.00 | C |
| ATOM | 6656 | N | LEU | A 847 | 34.851 | 64.260 | −22.441 | 1.00 | 0.00 | N |
| ATOM | 6657 | CA | LEU | A 847 | 35.922 | 64.023 | −21.472 | 1.00 | 0.00 | C |
| ATOM | 6658 | C | LEU | A 847 | 36.216 | 65.349 | −20.773 | 1.00 | 0.00 | C |
| ATOM | 6659 | O | LEU | A 847 | 36.143 | 66.410 | −21.400 | 1.00 | 0.00 | O |
| ATOM | 6660 | CB | LEU | A 847 | 37.192 | 63.512 | −22.172 | 1.00 | 0.00 | C |
| ATOM | 6661 | CG | LEU | A 847 | 37.171 | 62.165 | −22.916 | 1.00 | 0.00 | C |
| ATOM | 6662 | CD1 | LEU | A 847 | 36.466 | 61.120 | −22.060 | 1.00 | 0.00 | C |
| ATOM | 6663 | CD2 | LEU | A 847 | 36.471 | 62.316 | −24.262 | 1.00 | 0.00 | C |
| ATOM | 6664 | N | THR | A 848 | 36.561 | 65.290 | −19.485 | 1.00 | 0.00 | N |
| ATOM | 6665 | CA | THR | A 848 | 36.829 | 66.506 | −18.720 | 1.00 | 0.00 | C |
| ATOM | 6666 | C | THR | A 848 | 38.236 | 66.591 | −18.144 | 1.00 | 0.00 | C |
| ATOM | 6667 | O | THR | A 848 | 38.915 | 65.576 | −17.958 | 1.00 | 0.00 | O |
| ATOM | 6668 | CB | THR | A 848 | 35.864 | 66.640 | −17.533 | 1.00 | 0.00 | C |
| ATOM | 6669 | OG1 | THR | A 848 | 36.274 | 65.750 | −16.486 | 1.00 | 0.00 | O |
| ATOM | 6670 | CG2 | THR | A 848 | 34.448 | 66.297 | −17.956 | 1.00 | 0.00 | C |
| ATOM | 6671 | N | GLY | A 849 | 38.661 | 67.820 | −17.862 | 1.00 | 0.00 | N |
| ATOM | 6672 | CA | GLY | A 849 | 39.969 | 68.042 | −17.280 | 1.00 | 0.00 | C |
| ATOM | 6673 | C | GLY | A 849 | 39.790 | 68.414 | −15.820 | 1.00 | 0.00 | C |
| ATOM | 6674 | O | GLY | A 849 | 40.715 | 68.890 | −15.159 | 1.00 | 0.00 | O |
| ATOM | 6675 | N | GLN | A 850 | 38.579 | 68.197 | −15.321 | 1.00 | 0.00 | N |
| ATOM | 6676 | CA | GLN | A 850 | 38.247 | 68.499 | −13.936 | 1.00 | 0.00 | C |
| ATOM | 6677 | C | GLN | A 850 | 36.964 | 67.769 | −13.548 | 1.00 | 0.00 | C |
| ATOM | 6678 | O | GLN | A 850 | 36.091 | 67.548 | −14.388 | 1.00 | 0.00 | O |
| ATOM | 6679 | CB | GLN | A 850 | 38.048 | 70.010 | −13.753 | 1.00 | 0.00 | C |
| ATOM | 6680 | CG | GLN | A 850 | 36.894 | 70.622 | −14.571 | 1.00 | 0.00 | C |
| ATOM | 6681 | CD | GLN | A 850 | 37.199 | 70.719 | −16.053 | 1.00 | 0.00 | C |
| ATOM | 6682 | OE1 | GLN | A 850 | 38.296 | 71.125 | −16.447 | 1.00 | 0.00 | O |
| ATOM | 6683 | NE2 | GLN | A 850 | 36.221 | 70.361 | −16.890 | 1.00 | 0.00 | N |
| ATOM | 6684 | N | PRO | A 851 | 36.838 | 67.375 | −12.269 | 1.00 | 0.00 | N |
| ATOM | 6685 | CA | PRO | A 851 | 35.623 | 66.677 | −11.838 | 1.00 | 0.00 | C |
| ATOM | 6686 | C | PRO | A 851 | 34.485 | 67.674 | −11.637 | 1.00 | 0.00 | C |
| ATOM | 6687 | O | PRO | A 851 | 34.672 | 68.731 | −11.019 | 1.00 | 0.00 | O |
| ATOM | 6688 | CB | PRO | A 851 | 36.049 | 66.010 | −10.532 | 1.00 | 0.00 | C |
| ATOM | 6689 | CG | PRO | A 851 | 37.026 | 67.011 | −9.962 | 1.00 | 0.00 | C |
| ATOM | 6690 | CD | PRO | A 851 | 37.839 | 67.418 | −11.188 | 1.00 | 0.00 | C |
| ATOM | 6691 | N | LEU | A 852 | 33.318 | 67.331 | −12.173 | 1.00 | 0.00 | N |
| ATOM | 6692 | CA | LEU | A 852 | 32.124 | 68.168 | −12.079 | 1.00 | 0.00 | C |
| ATOM | 6693 | C | LEU | A 852 | 30.904 | 67.250 | −12.016 | 1.00 | 0.00 | C |
| ATOM | 6694 | O | LEU | A 852 | 31.017 | 66.048 | −12.253 | 1.00 | 0.00 | O |
| ATOM | 6695 | CB | LEU | A 852 | 32.017 | 69.077 | −13.311 | 1.00 | 0.00 | C |
| ATOM | 6696 | CG | LEU | A 852 | 33.138 | 70.100 | −13.532 | 1.00 | 0.00 | C |
| ATOM | 6697 | CD1 | LEU | A 852 | 33.032 | 70.709 | −14.928 | 1.00 | 0.00 | C |
| ATOM | 6698 | CD2 | LEU | A 852 | 33.038 | 71.189 | −12.470 | 1.00 | 0.00 | C |
| ATOM | 6699 | N | GLY | A 853 | 29.741 | 67.810 | −11.696 | 1.00 | 0.00 | N |
| ATOM | 6700 | CA | GLY | A 853 | 28.540 | 66.997 | −11.626 | 1.00 | 0.00 | C |
| ATOM | 6701 | C | GLY | A 853 | 27.796 | 66.992 | −12.952 | 1.00 | 0.00 | C |
| ATOM | 6702 | O | GLY | A 853 | 27.826 | 67.978 | −13.688 | 1.00 | 0.00 | O |
| ATOM | 6703 | N | GLY | A 854 | 27.118 | 65.892 | −13.265 | 1.00 | 0.00 | N |
| ATOM | 6704 | CA | GLY | A 854 | 26.400 | 65.839 | −14.527 | 1.00 | 0.00 | C |
| ATOM | 6705 | C | GLY | A 854 | 25.349 | 64.756 | −14.621 | 1.00 | 0.00 | C |
| ATOM | 6706 | O | GLY | A 854 | 25.146 | 63.990 | −13.678 | 1.00 | 0.00 | O |
| ATOM | 6707 | N | SER | A 855 | 24.685 | 64.682 | −15.772 | 1.00 | 0.00 | N |
| ATOM | 6708 | CA | SER | A 855 | 23.637 | 63.687 | −15.979 | 1.00 | 0.00 | C |
| ATOM | 6709 | C | SER | A 855 | 23.168 | 63.677 | −17.427 | 1.00 | 0.00 | C |
| ATOM | 6710 | O | SER | A 855 | 23.727 | 64.364 | −18.284 | 1.00 | 0.00 | O |
| ATOM | 6711 | CB | SER | A 855 | 22.442 | 64.006 | −15.070 | 1.00 | 0.00 | C |
| ATOM | 6712 | OG | SER | A 855 | 21.404 | 63.046 | −15.198 | 1.00 | 0.00 | O |
| ATOM | 6713 | N | SER | A 856 | 22.144 | 62.866 | −17.678 | 1.00 | 0.00 | N |
| ATOM | 6714 | CA | SER | A 856 | 21.494 | 62.755 | −18.980 | 1.00 | 0.00 | C |
| ATOM | 6715 | C | SER | A 856 | 20.027 | 62.753 | −18.564 | 1.00 | 0.00 | C |
| ATOM | 6716 | O | SER | A 856 | 19.470 | 61.704 | −18.224 | 1.00 | 0.00 | O |
| ATOM | 6717 | CB | SER | A 856 | 21.840 | 61.434 | −19.668 | 1.00 | 0.00 | C |
| ATOM | 6718 | OG | SER | A 856 | 21.180 | 61.335 | −20.920 | 1.00 | 0.00 | O |
| ATOM | 6719 | N | LEU | A 857 | 19.408 | 63.930 | −18.569 | 1.00 | 0.00 | N |
| ATOM | 6720 | CA | LEU | A 857 | 18.026 | 64.045 | −18.121 | 1.00 | 0.00 | C |
| ATOM | 6721 | C | LEU | A 857 | 16.964 | 63.699 | −19.155 | 1.00 | 0.00 | C |
| ATOM | 6722 | O | LEU | A 857 | 15.780 | 63.644 | −18.833 | 1.00 | 0.00 | O |
| ATOM | 6723 | CB | LEU | A 857 | 17.787 | 65.449 | −17.558 | 1.00 | 0.00 | C |
| ATOM | 6724 | CG | LEU | A 857 | 18.652 | 65.779 | −16.338 | 1.00 | 0.00 | C |
| ATOM | 6725 | CD1 | LEU | A 857 | 18.408 | 67.212 | −15.909 | 1.00 | 0.00 | C |
| ATOM | 6726 | CD2 | LEU | A 857 | 18.327 | 64.815 | −15.194 | 1.00 | 0.00 | C |
| ATOM | 6727 | N | ALA | A 858 | 17.392 | 63.459 | −20.386 | 1.00 | 0.00 | N |
| ATOM | 6728 | CA | ALA | A 858 | 16.483 | 63.097 | −21.466 | 1.00 | 0.00 | C |
| ATOM | 6729 | C | ALA | A 858 | 17.293 | 62.348 | −22.510 | 1.00 | 0.00 | C |
| ATOM | 6730 | O | ALA | A 858 | 18.498 | 62.560 | −22.634 | 1.00 | 0.00 | O |
| ATOM | 6731 | CB | ALA | A 858 | 15.849 | 64.355 | −22.085 | 1.00 | 0.00 | C |
| ATOM | 6732 | N | SER | A 859 | 16.633 | 61.461 | −23.247 | 1.00 | 0.00 | N |
| ATOM | 6733 | CA | SER | A 859 | 17.302 | 60.686 | −24.280 | 1.00 | 0.00 | C |
| ATOM | 6734 | C | SER | A 859 | 18.071 | 61.620 | −25.209 | 1.00 | 0.00 | C |
| ATOM | 6735 | O | SER | A 859 | 17.558 | 62.668 | −25.612 | 1.00 | 0.00 | O |
| ATOM | 6736 | CB | SER | A 859 | 16.272 | 59.882 | −25.084 | 1.00 | 0.00 | C |
| ATOM | 6737 | OG | SER | A 859 | 16.896 | 59.140 | −26.117 | 1.00 | 0.00 | O |
| ATOM | 6738 | N | GLY | A 860 | 19.303 | 61.240 | −25.534 | 1.00 | 0.00 | N |
| ATOM | 6739 | CA | GLY | A 860 | 20.131 | 62.041 | −26.420 | 1.00 | 0.00 | C |
| ATOM | 6740 | C | GLY | A 860 | 20.837 | 63.227 | −25.781 | 1.00 | 0.00 | C |
| ATOM | 6741 | O | GLY | A 860 | 21.551 | 63.969 | −26.463 | 1.00 | 0.00 | O |
| ATOM | 6742 | N | GLU | A 861 | 20.660 | 63.412 | −24.479 | 1.00 | 0.00 | N |
| ATOM | 6743 | CA | GLU | A 861 | 21.292 | 64.538 | −23.798 | 1.00 | 0.00 | C |
| ATOM | 6744 | C | GLU | A 861 | 22.442 | 64.188 | −22.863 | 1.00 | 0.00 | C |
| ATOM | 6745 | O | GLU | A 861 | 22.530 | 63.078 | −22.338 | 1.00 | 0.00 | O |
| ATOM | 6746 | CB | GLU | A 861 | 20.261 | 65.316 | −22.978 | 1.00 | 0.00 | C |
| ATOM | 6747 | CG | GLU | A 861 | 19.194 | 66.041 | −23.775 | 1.00 | 0.00 | C |
| ATOM | 6748 | CD | GLU | A 861 | 18.285 | 66.871 | −22.882 | 1.00 | 0.00 | C |
| ATOM | 6749 | OE1 | GLU | A 861 | 18.523 | 66.920 | −21.652 | 1.00 | 0.00 | O |
| ATOM | 6750 | OE2 | GLU | A 861 | 17.335 | 67.480 | −23.407 | 1.00 | 0.00 | O |
| ATOM | 6751 | N | LEU | A 862 | 23.316 | 65.168 | −22.669 | 1.00 | 0.00 | N |
| ATOM | 6752 | CA | LEU | A 862 | 24.442 | 65.071 | −21.745 | 1.00 | 0.00 | C |
| ATOM | 6753 | C | LEU | A 862 | 24.518 | 66.469 | −21.160 | 1.00 | 0.00 | C |
| ATOM | 6754 | O | LEU | A 862 | 24.315 | 67.452 | −21.873 | 1.00 | 0.00 | O |
| ATOM | 6755 | CB | LEU | A 862 | 25.765 | 64.773 | −22.459 | 1.00 | 0.00 | C |
| ATOM | 6756 | CG | LEU | A 862 | 26.099 | 63.390 | −23.029 | 1.00 | 0.00 | C |
| ATOM | 6757 | CD1 | LEU | A 862 | 27.461 | 63.457 | −23.706 | 1.00 | 0.00 | C |
| ATOM | 6758 | CD2 | LEU | A 862 | 26.114 | 62.345 | −21.929 | 1.00 | 0.00 | C |
| ATOM | 6759 | N | GLU | A 863 | 24.774 | 66.575 | −19.865 | 1.00 | 0.00 | N |
| ATOM | 6760 | CA | GLU | A 863 | 24.904 | 67.893 | −19.273 | 1.00 | 0.00 | C |
| ATOM | 6761 | C | GLU | A 863 | 25.852 | 67.818 | −18.093 | 1.00 | 0.00 | C |
| ATOM | 6762 | O | GLU | A 863 | 25.915 | 66.806 | −17.392 | 1.00 | 0.00 | O |
| ATOM | 6763 | CB | GLU | A 863 | 23.536 | 68.452 | −18.862 | 1.00 | 0.00 | C |
| ATOM | 6764 | CG | GLU | A 863 | 23.017 | 68.039 | −17.503 | 1.00 | 0.00 | C |
| ATOM | 6765 | CD | GLU | A 863 | 21.689 | 68.706 | −17.187 | 1.00 | 0.00 | C |
| ATOM | 6766 | OE1 | GLU | A 863 | 21.534 | 69.221 | −16.061 | 1.00 | 0.00 | O |
| ATOM | 6767 | OE2 | GLU | A 863 | 20.796 | 69.712 | −18.066 | 1.00 | 0.00 | O |
| ATOM | 6768 | N | ILE | A 864 | 26.603 | 68.891 | −17.889 | 1.00 | 0.00 | N |
| ATOM | 6769 | CA | ILE | A 864 | 27.575 | 68.939 | −16.811 | 1.00 | 0.00 | C |
| ATOM | 6770 | C | ILE | A 864 | 27.625 | 70.360 | −16.241 | 1.00 | 0.00 | C |
| ATOM | 6771 | O | ILE | A 864 | 27.744 | 71.335 | −16.985 | 1.00 | 0.00 | O |
| ATOM | 6772 | CB | ILE | A 864 | 28.955 | 68.478 | −17.350 | 1.00 | 0.00 | C |
| ATOM | 6773 | CG1 | ILE | A 864 | 29.982 | 68.419 | −16.222 | 1.00 | 0.00 | C |
| ATOM | 6774 | CG2 | ILE | A 864 | 29.402 | 69.389 | −18.499 | 1.00 | 0.00 | C |
| ATOM | 6775 | CD1 | ILE | A 864 | 31.263 | 67.713 | −16.629 | 1.00 | 0.00 | C |
| ATOM | 6776 | N | MET | A 865 | 27.510 | 70.463 | −14.920 | 1.00 | 0.00 | N |
| ATOM | 6777 | CA | MET | A 865 | 27.507 | 71.750 | −14.227 | 1.00 | 0.00 | C |
| ATOM | 6778 | C | MET | A 865 | 28.875 | 72.436 | −14.272 | 1.00 | 0.00 | C |
| ATOM | 6779 | O | MET | A 865 | 29.907 | 71.792 | −14.084 | 1.00 | 0.00 | O |
| ATOM | 6780 | CB | MET | A 865 | 27.080 | 71.551 | −12.770 | 1.00 | 0.00 | C |
| ATOM | 6781 | CG | MET | A 865 | 26.489 | 72.792 | −12.129 | 1.00 | 0.00 | C |
| ATOM | 6782 | SD | MET | A 865 | 24.888 | 73.177 | −12.876 | 1.00 | 0.00 | S |
| ATOM | 6783 | CE | MET | A 865 | 23.854 | 71.946 | −12.056 | 1.00 | 0.00 | C |
| ATOM | 6784 | N | GLN | A 866 | 28.870 | 73.748 | −14.498 | 1.00 | 0.00 | N |
| ATOM | 6785 | CA | GLN | A 866 | 30.106 | 74.521 | −14.590 | 1.00 | 0.00 | C |
| ATOM | 6786 | C | GLN | A 866 | 30.503 | 75.186 | −13.277 | 1.00 | 0.00 | C |
| ATOM | 6787 | O | GLN | A 866 | 31.675 | 75.190 | −12.909 | 1.00 | 0.00 | O |
| ATOM | 6788 | CB | GLN | A 866 | 29.970 | 75.571 | −15.692 | 1.00 | 0.00 | C |
| ATOM | 6789 | CG | GLN | A 866 | 29.633 | 74.961 | −17.037 | 1.00 | 0.00 | C |
| ATOM | 6790 | CD | GLN | A 866 | 30.642 | 73.906 | −17.453 | 1.00 | 0.00 | C |
| ATOM | 6791 | OE1 | GLN | A 866 | 31.800 | 74.217 | −17.751 | 1.00 | 0.00 | O |
| ATOM | 6792 | NE2 | GLN | A 866 | 30.210 | 72.647 | −17.463 | 1.00 | 0.00 | N |
| ATOM | 6793 | N | ASP | A 867 | 29.526 | 75.769 | −12.591 | 1.00 | 0.00 | N |
| ATOM | 6794 | CA | ASP | A 867 | 29.761 | 76.404 | −11.301 | 1.00 | 0.00 | C |
| ATOM | 6795 | C | ASP | A 867 | 28.409 | 76.665 | −10.664 | 1.00 | 0.00 | C |
| ATOM | 6796 | O | ASP | A 867 | 27.372 | 76.625 | −11.336 | 1.00 | 0.00 | O |
| ATOM | 6797 | CB | ASP | A 867 | 30.531 | 77.722 | −11.453 | 1.00 | 0.00 | C |
| ATOM | 6798 | CG | ASP | A 867 | 31.201 | 78.165 | −10.152 | 1.00 | 0.00 | C |
| ATOM | 6799 | OD1 | ASP | A 867 | 31.022 | 77.490 | −9.114 | 1.00 | 0.00 | O |
| ATOM | 6800 | OD2 | ASP | A 867 | 31.912 | 79.188 | −10.170 | 1.00 | 0.00 | O |
| ATOM | 6801 | N | ARG | A 868 | 28.425 | 76.920 | −9.363 | 1.00 | 0.00 | N |
| ATOM | 6802 | CA | ARG | A 868 | 27.206 | 77.181 | −8.620 | 1.00 | 0.00 | C |
| ATOM | 6803 | C | ARG | A 868 | 27.513 | 78.188 | −7.522 | 1.00 | 0.00 | C |
| ATOM | 6804 | O | ARG | A 868 | 28.539 | 78.088 | −6.848 | 1.00 | 0.00 | O |
| ATOM | 6805 | CB | ARG | A 868 | 26.679 | 75.872 | −8.022 | 1.00 | 0.00 | C |
| ATOM | 6806 | CG | ARG | A 868 | 27.691 | 75.120 | −7.166 | 1.00 | 0.00 | C |
| ATOM | 6807 | CD | ARG | A 868 | 27.391 | 73.618 | −7.180 | 1.00 | 0.00 | C |
| ATOM | 6808 | NE | ARG | A 868 | 26.037 | 73.333 | −6.722 | 1.00 | 0.00 | N |
| ATOM | 6809 | CZ | ARG | A 868 | 25.715 | 73.041 | −5.466 | 1.00 | 0.00 | C |
| ATOM | 6810 | NH1 | ARG | A 868 | 26.655 | 72.984 | −4.530 | 1.00 | 0.00 | N |
| ATOM | 6811 | NH2 | ARG | A 868 | 24.448 | 72.821 | −5.142 | 1.00 | 0.00 | N |
| ATOM | 6812 | N | ARG | A 869 | 26.625 | 79.163 | −7.362 | 1.00 | 0.00 | N |
| ATOM | 6813 | CA | ARG | A 869 | 26.776 | 80.208 | −6.352 | 1.00 | 0.00 | C |
| ATOM | 6814 | C | ARG | A 869 | 25.497 | 80.162 | −5.519 | 1.00 | 0.00 | C |
| ATOM | 6815 | O | ARG | A 869 | 24.416 | 80.477 | −6.013 | 1.00 | 0.00 | O |
| ATOM | 6816 | CB | ARG | A 869 | 26.938 | 81.572 | −7.038 | 1.00 | 0.00 | C |
| ATOM | 6817 | CG | ARG | A 869 | 27.263 | 82.741 | −6.107 | 1.00 | 0.00 | C |
| ATOM | 6818 | CD | ARG | A 869 | 27.510 | 84.029 | −6.898 | 1.00 | 0.00 | C |
| ATOM | 6819 | NE | ARG | A 869 | 27.690 | 85.197 | −6.032 | 1.00 | 0.00 | N |
| ATOM | 6820 | CZ | ARG | A 869 | 28.813 | 85.494 | −5.383 | 1.00 | 0.00 | C |
| ATOM | 6821 | NH1 | ARG | A 869 | 29.879 | 84.717 | −5.495 | 1.00 | 0.00 | N |
| ATOM | 6822 | NH2 | ARG | A 869 | 28.867 | 86.572 | −4.609 | 1.00 | 0.00 | N |
| ATOM | 6823 | N | LEU | A 870 | 25.631 | 79.749 | −4.262 | 1.00 | 0.00 | N |
| ATOM | 6824 | CA | LEU | A 870 | 24.493 | 79.614 | −3.358 | 1.00 | 0.00 | C |
| ATOM | 6825 | C | LEU | A 870 | 24.591 | 80.533 | −2.142 | 1.00 | 0.00 | C |
| ATOM | 6826 | O | LEU | A 870 | 25.586 | 80.524 | −1.417 | 1.00 | 0.00 | O |
| ATOM | 6827 | CB | LEU | A 870 | 24.392 | 78.157 | −2.911 | 1.00 | 0.00 | C |
| ATOM | 6828 | CG | LEU | A 870 | 24.393 | 77.210 | −4.116 | 1.00 | 0.00 | C |
| ATOM | 6829 | CD1 | LEU | A 870 | 24.766 | 75.819 | −3.678 | 1.00 | 0.00 | C |
| ATOM | 6830 | CD2 | LEU | A 870 | 23.029 | 77.237 | −4.795 | 1.00 | 0.00 | C |
| ATOM | 6831 | N | ALA | A 871 | 23.537 | 81.311 | −1.916 | 1.00 | 0.00 | N |
| ATOM | 6832 | CA | ALA | A 871 | 23.509 | 82.261 | −0.811 | 1.00 | 0.00 | C |
| ATOM | 6833 | C | ALA | A 871 | 23.249 | 81.660 | 0.567 | 1.00 | 0.00 | C |
| ATOM | 6834 | O | ALA | A 871 | 23.665 | 82.226 | 1.578 | 1.00 | 0.00 | O |
| ATOM | 6835 | CB | ALA | A 871 | 22.476 | 83.352 | −1.099 | 1.00 | 0.00 | C |
| ATOM | 6836 | N | SER | A 872 | 22.573 | 80.519 | 0.624 | 1.00 | 0.00 | N |
| ATOM | 6837 | CA | SER | A 872 | 22.272 | 79.924 | 1.920 | 1.00 | 0.00 | C |
| ATOM | 6838 | C | SER | A 872 | 23.032 | 78.644 | 2.244 | 1.00 | 0.00 | C |
| ATOM | 6839 | O | SER | A 872 | 23.603 | 77.996 | 1.367 | 1.00 | 0.00 | O |
| ATOM | 6840 | CB | SER | A 872 | 20.768 | 79.667 | 2.037 | 1.00 | 0.00 | C |
| ATOM | 6841 | OG | SER | A 872 | 20.331 | 78.753 | 1.049 | 1.00 | 0.00 | O |
| ATOM | 6842 | N | ASP | A 873 | 23.034 | 78.304 | 3.528 | 1.00 | 0.00 | N |
| ATOM | 6843 | CA | ASP | A 873 | 23.690 | 77.105 | 4.034 | 1.00 | 0.00 | C |
| ATOM | 6844 | C | ASP | A 873 | 22.696 | 75.950 | 3.952 | 1.00 | 0.00 | C |
| ATOM | 6845 | O | ASP | A 873 | 21.498 | 76.154 | 4.143 | 1.00 | 0.00 | O |
| ATOM | 6846 | CG | ASP | A 873 | 24.114 | 77.330 | 5.487 | 1.00 | 0.00 | C |
| ATOM | 6847 | CG | ASP | A 873 | 24.577 | 76.060 | 6.161 | 1.00 | 0.00 | C |
| ATOM | 6848 | OD1 | ASP | A 873 | 23.809 | 75.503 | 6.972 | 1.00 | 0.00 | O |
| ATOM | 6849 | OD2 | ASP | A 873 | 25.707 | 75.613 | 5.869 | 1.00 | 0.00 | O |
| ATOM | 6850 | N | ASP | A 874 | 23.183 | 74.743 | 3.666 | 1.00 | 0.00 | N |
| ATOM | 6851 | CA | ASP | A 874 | 22.299 | 73.587 | 3.559 | 1.00 | 0.00 | C |
| ATOM | 6852 | C | ASP | A 874 | 22.250 | 72.685 | 4.796 | 1.00 | 0.00 | C |
| ATOM | 6853 | O | ASP | A 874 | 22.034 | 71.479 | 4.695 | 1.00 | 0.00 | O |
| ATOM | 6854 | CB | ASP | A 874 | 22.634 | 72.763 | 2.305 | 1.00 | 0.00 | C |
| ATOM | 6855 | CG | ASP | A 874 | 24.121 | 72.503 | 2.146 | 1.00 | 0.00 | C |
| ATOM | 6856 | OD1 | ASP | A 874 | 24.902 | 72.863 | 3.056 | 1.00 | 0.00 | O |
| ATOM | 6857 | OD2 | ASP | A 874 | 24.505 | 71.932 | 1.101 | 1.00 | 0.00 | O |
| ATOM | 6858 | N | GLU | A 875 | 22.458 | 73.289 | 5.961 | 1.00 | 0.00 | N |
| ATOM | 6859 | CA | GLU | A 875 | 22.382 | 72.602 | 7.247 | 1.00 | 0.00 | C |
| ATOM | 6860 | C | GLU | A 875 | 23.153 | 71.300 | 7.458 | 1.00 | 0.00 | C |
| ATOM | 6861 | O | GLU | A 875 | 22.632 | 70.374 | 8.081 | 1.00 | 0.00 | O |
| ATOM | 6862 | CB | GLU | A 875 | 20.911 | 72.352 | 7.595 | 1.00 | 0.00 | C |
| ATOM | 6863 | CG | GLU | A 875 | 20.041 | 73.600 | 7.577 | 1.00 | 0.00 | C |
| ATOM | 6864 | CD | GLU | A 875 | 18.589 | 73.296 | 7.895 | 1.00 | 0.00 | C |
| ATOM | 6865 | OE1 | GLU | A 875 | 18.305 | 72.852 | 9.026 | 1.00 | 0.00 | O |
| ATOM | 6866 | OE2 | GLU | A 875 | 17.731 | 73.494 | 7.010 | 1.00 | 0.00 | O |
| ATOM | 6867 | N | ARG | A 876 | 24.380 | 71.213 | 6.959 | 1.00 | 0.00 | N |
| ATOM | 6868 | CA | ARG | A 876 | 25.163 | 70.002 | 7.180 | 1.00 | 0.00 | C |
| ATOM | 6869 | C | ARG | A 876 | 26.423 | 70.305 | 7.993 | 1.00 | 0.00 | C |
| ATOM | 6870 | O | ARG | A 876 | 27.317 | 69.465 | 8.112 | 1.00 | 0.00 | O |
| ATOM | 6871 | CB | ARG | A 876 | 25.518 | 69.326 | 5.847 | 1.00 | 0.00 | C |
| ATOM | 6872 | CG | ARG | A 876 | 24.314 | 68.689 | 5.130 | 1.00 | 0.00 | C |
| ATOM | 6873 | CD | ARG | A 876 | 23.608 | 67.662 | 6.024 | 1.00 | 0.00 | C |
| ATOM | 6874 | NE | ARG | A 876 | 22.519 | 66.956 | 5.346 | 1.00 | 0.00 | N |
| ATOM | 6875 | CZ | ARG | A 876 | 21.357 | 67.507 | 4.996 | 1.00 | 0.00 | C |
| ATOM | 6876 | NH1 | ARG | A 876 | 21.111 | 68.786 | 5.254 | 1.00 | 0.00 | N |
| ATOM | 6877 | NH2 | ARG | A 876 | 20.434 | 66.770 | 4.388 | 1.00 | 0.00 | N |
| ATOM | 6878 | N | GLY | A 877 | 26.486 | 71.512 | 8.554 | 1.00 | 0.00 | N |
| ATOM | 6879 | CA | GLY | A 877 | 27.624 | 71.886 | 9.376 | 1.00 | 0.00 | C |
| ATOM | 6880 | C | GLY | A 877 | 28.606 | 72.910 | 8.831 | 1.00 | 0.00 | C |
| ATOM | 6881 | O | GLY | A 877 | 29.417 | 73.448 | 9.591 | 1.00 | 0.00 | O |
| ATOM | 6882 | N | LEU | A 878 | 28.545 | 73.193 | 7.534 | 1.00 | 0.00 | N |
| ATOM | 6883 | CA | LEU | A 878 | 29.465 | 74.155 | 6.931 | 1.00 | 0.00 | C |
| ATOM | 6884 | C | LEU | A 878 | 29.274 | 75.567 | 7.490 | 1.00 | 0.00 | C |
| ATOM | 6885 | O | LEU | A 878 | 30.242 | 76.307 | 7.671 | 1.00 | 0.00 | O |
| ATOM | 6886 | CB | LEU | A 878 | 29.302 | 74.157 | 5.409 | 1.00 | 0.00 | C |
| ATOM | 6887 | CG | LEU | A 878 | 30.187 | 75.112 | 4.601 | 1.00 | 0.00 | C |
| ATOM | 6888 | CD1 | LEU | A 878 | 31.649 | 74.944 | 4.999 | 1.00 | 0.00 | C |
| ATOM | 6889 | CD2 | LEU | A 878 | 30.003 | 74.835 | 3.120 | 1.00 | 0.00 | C |
| ATOM | 6890 | N | GLY | A 879 | 28.026 | 75.938 | 7.756 | 1.00 | 0.00 | N |
| ATOM | 6891 | CA | GLY | A 879 | 27.758 | 77.253 | 8.315 | 1.00 | 0.00 | C |
| ATOM | 6892 | C | GLY | A 879 | 27.829 | 78.416 | 7.343 | 1.00 | 0.00 | C |
| ATOM | 6893 | O | GLY | A 879 | 27.911 | 79.571 | 7.762 | 1.00 | 0.00 | O |
| ATOM | 6894 | N | GLN | A 880 | 27.807 | 78.119 | 6.048 | 1.00 | 0.00 | N |
| ATOM | 6895 | CA | GLN | A 880 | 27.842 | 79.160 | 5.029 | 1.00 | 0.00 | C |
| ATOM | 6896 | C | GLN | A 880 | 27.423 | 78.614 | 3.672 | 1.00 | 0.00 | C |
| ATOM | 6897 | O | GLN | A 880 | 27.382 | 77.399 | 3.457 | 1.00 | 0.00 | O |
| ATOM | 6898 | CB | GLN | A 880 | 29.248 | 79.772 | 4.907 | 1.00 | 0.00 | C |
| ATOM | 6899 | CG | GLN | A 880 | 30.364 | 78.772 | 4.582 | 1.00 | 0.00 | C |
| ATOM | 6900 | CD | GLN | A 880 | 31.593 | 79.431 | 3.966 | 1.00 | 0.00 | C |
| ATOM | 6901 | OE1 | GLN | A 880 | 31.621 | 79.729 | 2.767 | 1.00 | 0.00 | O |
| ATOM | 6902 | NE2 | GLN | A 880 | 32.609 | 79.671 | 4.783 | 1.00 | 0.00 | N |
| ATOM | 6903 | N | GLY | A 881 | 27.096 | 79.527 | 2.765 | 1.00 | 0.00 | N |
| ATOM | 6904 | CA | GLY | A 881 | 26.729 | 79.130 | 1.423 | 1.00 | 0.00 | C |
| ATOM | 6905 | C | GLY | A 881 | 28.013 | 79.148 | 0.615 | 1.00 | 0.00 | C |
| ATOM | 6906 | O | GLY | A 881 | 29.107 | 79.119 | 1.179 | 1.00 | 0.00 | O |
| ATOM | 6907 | N | VAL | A 882 | 27.887 | 79.199 | −0.703 | 1.00 | 0.00 | N |
| ATOM | 6908 | CA | VAL | A 882 | 29.046 | 79.234 | −1.575 | 1.00 | 0.00 | C |
| ATOM | 6909 | C | VAL | A 882 | 28.999 | 80.556 | −2.328 | 1.00 | 0.00 | C |
| ATOM | 6910 | O | VAL | A 882 | 28.313 | 80.680 | −3.340 | 1.00 | 0.00 | O |
| ATOM | 6911 | CB | VAL | A 882 | 29.018 | 78.060 | −2.570 | 1.00 | 0.00 | C |
| ATOM | 6912 | CG1 | VAL | A 882 | 30.230 | 78.117 | −3.480 | 1.00 | 0.00 | C |
| ATOM | 6913 | CG2 | VAL | A 882 | 28.988 | 76.744 | −1.804 | 1.00 | 0.00 | C |
| ATOM | 6914 | N | LEU | A 883 | 29.726 | 81.541 | −1.812 | 1.00 | 0.00 | N |
| ATOM | 6915 | CA | LEU | A 883 | 29.761 | 82.871 | −2.410 | 1.00 | 0.00 | C |
| ATOM | 6916 | C | LEU | A 883 | 31.184 | 83.330 | −2.720 | 1.00 | 0.00 | C |
| ATOM | 6917 | O | LEU | A 883 | 31.421 | 84.517 | −2.942 | 1.00 | 0.00 | O |
| ATOM | 6918 | CB | LEU | A 883 | 29.093 | 83.874 | −1.461 | 1.00 | 0.00 | C |
| ATOM | 6919 | CG | LEU | A 883 | 27.586 | 83.722 | −1.219 | 1.00 | 0.00 | C |
| ATOM | 6920 | CD1 | LEU | A 883 | 27.162 | 84.533 | −0.003 | 1.00 | 0.00 | C |
| ATOM | 6921 | CD2 | LEU | A 883 | 26.832 | 84.189 | −2.448 | 1.00 | 0.00 | C |
| ATOM | 6922 | N | ASP | A 884 | 32.125 | 82.391 | −2.747 | 1.00 | 0.00 | N |
| ATOM | 6923 | CA | ASP | A 884 | 33.521 | 82.714 | −3.019 | 1.00 | 0.00 | C |
| ATOM | 6924 | C | ASP | A 884 | 33.993 | 82.266 | −4.398 | 1.00 | 0.00 | C |
| ATOM | 6925 | O | ASP | A 884 | 35.167 | 81.949 | −4.594 | 1.00 | 0.00 | O |
| ATOM | 6926 | CB | ASP | A 884 | 34.422 | 82.106 | −1.938 | 1.00 | 0.00 | C |
| ATOM | 6927 | CG | ASP | A 884 | 34.181 | 80.621 | −1.738 | 1.00 | 0.00 | C |
| ATOM | 6928 | OD1 | ASP | A 884 | 34.836 | 80.039 | −0.847 | 1.00 | 0.00 | O |
| ATOM | 6929 | OD2 | ASP | A 884 | 33.344 | 80.037 | −2.465 | 1.00 | 0.00 | O |
| ATOM | 6930 | N | ASN | A 885 | 33.070 | 82.256 | −5.353 | 1.00 | 0.00 | N |
| ATOM | 6931 | CA | ASN | A 885 | 33.373 | 81.862 | −6.723 | 1.00 | 0.00 | C |
| ATOM | 6932 | C | ASN | A 885 | 34.459 | 82.741 | −7.318 | 1.00 | 0.00 | C |
| ATOM | 6933 | O | ASN | A 885 | 34.588 | 83.913 | −6.966 | 1.00 | 0.00 | O |
| ATOM | 6934 | CB | ASN | A 885 | 32.121 | 81.990 | −7.586 | 1.00 | 0.00 | C |
| ATOM | 6935 | CG | ASN | A 885 | 30.950 | 81.227 | −7.019 | 1.00 | 0.00 | C |
| ATOM | 6936 | OD1 | ASN | A 885 | 30.705 | 80.077 | −7.385 | 1.00 | 0.00 | O |
| ATOM | 6937 | ND2 | ASN | A 885 | 30.230 | 81.856 | −6.098 | 1.00 | 0.00 | N |
| ATOM | 6938 | N | LYS | A 886 | 35.231 | 82.165 | −8.230 | 1.00 | 0.00 | N |
| ATOM | 6939 | CA | LYS | A 886 | 36.289 | 82.892 | −8.910 | 1.00 | 0.00 | C |
| ATOM | 6940 | C | LYS | A 886 | 36.399 | 82.306 | −10.310 | 1.00 | 0.00 | C |
| ATOM | 6941 | O | LYS | A 886 | 36.049 | 81.146 | −10.534 | 1.00 | 0.00 | O |
| ATOM | 6942 | CB | LYS | A 886 | 37.614 | 82.747 | −8.152 | 1.00 | 0.00 | C |
| ATOM | 6943 | CG | LYS | A 886 | 38.104 | 81.318 | −8.014 | 1.00 | 0.00 | C |
| ATOM | 6944 | CD | LYS | A 886 | 39.238 | 81.206 | −7.001 | 1.00 | 0.00 | C |
| ATOM | 6945 | CE | LYS | A 886 | 40.440 | 82.042 | −7.405 | 1.00 | 0.00 | C |
| ATOM | 6946 | NZ | LYS | A 886 | 41.572 | 81.858 | −6.461 | 1.00 | 0.00 | N |
| ATOM | 6947 | N | PRO | A 887 | 36.871 | 83.104 | −11.279 | 1.00 | 0.00 | N |
| ATOM | 6948 | CA | PRO | A 887 | 37.002 | 82.608 | −12.651 | 1.00 | 0.00 | C |
| ATOM | 6949 | C | PRO | A 887 | 37.793 | 81.304 | −12.741 | 1.00 | 0.00 | C |
| ATOM | 6950 | O | PRO | A 887 | 38.850 | 81.161 | −12.129 | 1.00 | 0.00 | O |
| ATOM | 6951 | CB | PRO | A 887 | 37.704 | 83.761 | −13.366 | 1.00 | 0.00 | C |
| ATOM | 6952 | CG | PRO | A 887 | 37.185 | 84.967 | −12.627 | 1.00 | 0.00 | C |
| ATOM | 6953 | CD | PRO | A 887 | 37.259 | 84.524 | −11.189 | 1.00 | 0.00 | C |
| ATOM | 6954 | N | VAL | A 888 | 37.260 | 80.350 | −13.496 | 1.00 | 0.00 | N |
| ATOM | 6955 | CA | VAL | A 888 | 37.929 | 79.073 | −13.688 | 1.00 | 0.00 | C |
| ATOM | 6956 | C | VAL | A 888 | 37.830 | 78.679 | −15.156 | 1.00 | 0.00 | C |
| ATOM | 6957 | O | VAL | A 888 | 36.814 | 78.933 | −15.811 | 1.00 | 0.00 | O |
| ATOM | 6958 | CB | VAL | A 888 | 37.307 | 77.955 | −12.803 | 1.00 | 0.00 | C |
| ATOM | 6959 | CG1 | VAL | A 888 | 35.799 | 77.878 | −13.020 | 1.00 | 0.00 | C |
| ATOM | 6960 | CG2 | VAL | A 888 | 37.964 | 76.609 | −13.122 | 1.00 | 0.00 | C |
| ATOM | 6961 | N | LEU | A 889 | 38.897 | 78.081 | −15.675 | 1.00 | 0.00 | N |
| ATOM | 6962 | CA | LEU | A 889 | 38.914 | 77.640 | −17.061 | 1.00 | 0.00 | C |
| ATOM | 6963 | C | LEU | A 889 | 38.709 | 76.131 | −17.117 | 1.00 | 0.00 | C |
| ATOM | 6964 | O | LEU | A 889 | 39.626 | 75.368 | −16.817 | 1.00 | 0.00 | O |
| ATOM | 6965 | CB | LEU | A 889 | 40.248 | 77.984 | −17.734 | 1.00 | 0.00 | C |
| ATOM | 6966 | CG | LEU | A 889 | 40.306 | 77.575 | −19.215 | 1.00 | 0.00 | C |
| ATOM | 6967 | CD1 | LEU | A 889 | 39.375 | 78.476 | −20.023 | 1.00 | 0.00 | C |
| ATOM | 6968 | CD2 | LEU | A 889 | 41.734 | 77.677 | −19.740 | 1.00 | 0.00 | C |
| ATOM | 6969 | N | HIS | A 890 | 37.502 | 75.708 | −17.480 | 1.00 | 0.00 | N |
| ATOM | 6970 | CA | HIS | A 890 | 37.196 | 74.285 | −17.599 | 1.00 | 0.00 | C |
| ATOM | 6971 | C | HIS | A 890 | 37.584 | 73.830 | −18.993 | 1.00 | 0.00 | C |
| ATOM | 6972 | O | HIS | A 890 | 37.375 | 74.562 | −19.959 | 1.00 | 0.00 | O |
| ATOM | 6973 | CB | HIS | A 890 | 35.700 | 74.019 | −17.410 | 1.00 | 0.00 | C |
| ATOM | 6974 | CG | HIS | A 890 | 35.227 | 74.182 | −16.001 | 1.00 | 0.00 | C |
| ATOM | 6975 | ND1 | HIS | A 890 | 35.981 | 73.797 | −14.914 | 1.00 | 0.00 | N |
| ATOM | 6976 | CD2 | HIS | A 890 | 34.057 | 74.645 | −15.503 | 1.00 | 0.00 | C |
| ATOM | 6977 | CE1 | HIS | A 890 | 35.295 | 74.016 | −13.806 | 1.00 | 0.00 | C |
| ATOM | 6978 | NE2 | HIS | A 890 | 34.124 | 74.530 | −14.136 | 1.00 | 0.00 | N |
| ATOM | 6979 | N | ILE | A 891 | 38.134 | 72.625 | −19.107 | 1.00 | 0.00 | N |
| ATOM | 6980 | CA | ILE | A 891 | 38.530 | 72.110 | −20.412 | 1.00 | 0.00 | C |
| ATOM | 6981 | C | ILE | A 891 | 37.913 | 70.741 | −20.684 | 1.00 | 0.00 | C |
| ATOM | 6982 | O | ILE | A 891 | 37.681 | 69.959 | −19.760 | 1.00 | 0.00 | O |
| ATOM | 6983 | CB | ILE | A 891 | 40.067 | 72.025 | −20.537 | 1.00 | 0.00 | C |
| ATOM | 6984 | CG1 | ILE | A 891 | 40.632 | 71.016 | −19.531 | 1.00 | 0.00 | C |
| ATOM | 6985 | CG2 | ILE | A 891 | 40.677 | 73.405 | −20.283 | 1.00 | 0.00 | C |
| ATOM | 6986 | CD1 | ILE | A 891 | 42.125 | 70.735 | −19.727 | 1.00 | 0.00 | C |
| ATOM | 6987 | N | TYR | A 892 | 37.645 | 70.466 | −21.957 | 1.00 | 0.00 | N |
| ATOM | 6988 | CA | TYR | A 892 | 37.035 | 69.203 | −22.370 | 1.00 | 0.00 | C |
| ATOM | 6989 | C | TYR | A 892 | 37.446 | 68.804 | −23.778 | 1.00 | 0.00 | C |
| ATOM | 6990 | O | TYR | A 892 | 38.069 | 69.572 | −24.503 | 1.00 | 0.00 | O |
| ATOM | 6991 | CB | TYR | A 892 | 35.500 | 69.316 | −22.407 | 1.00 | 0.00 | C |
| ATOM | 6992 | CG | TYR | A 892 | 34.841 | 70.000 | −21.240 | 1.00 | 0.00 | C |
| ATOM | 6993 | CD1 | TYR | A 892 | 34.813 | 71.395 | −21.139 | 1.00 | 0.00 | C |
| ATOM | 6994 | CD2 | TYR | A 892 | 34.237 | 69.252 | −20.233 | 1.00 | 0.00 | C |
| ATOM | 6995 | CE1 | TYR | A 892 | 34.194 | 72.024 | −20.054 | 1.00 | 0.00 | C |
| ATOM | 6996 | CE2 | TYR | A 892 | 33.623 | 69.863 | −19.152 | 1.00 | 0.00 | C |
| ATOM | 6997 | CZ | TYR | A 892 | 33.602 | 71.246 | −19.064 | 1.00 | 0.00 | C |
| ATOM | 6998 | OH | TYR | A 892 | 32.990 | 71.836 | −17.984 | 1.00 | 0.00 | O |
| ATOM | 6999 | N | ARG | A 893 | 37.080 | 67.578 | −24.148 | 1.00 | 0.00 | N |
| ATOM | 7000 | CA | ARG | A 893 | 37.275 | 67.067 | −25.504 | 1.00 | 0.00 | C |
| ATOM | 7001 | C | ARG | A 893 | 35.900 | 66.469 | −25.808 | 1.00 | 0.00 | C |
| ATOM | 7002 | O | ARG | A 893 | 35.312 | 65.803 | −24.952 | 1.00 | 0.00 | O |
| ATOM | 7003 | CB | ARG | A 893 | 38.352 | 65.975 | −25.588 | 1.00 | 0.00 | C |
| ATOM | 7004 | CG | ARG | A 893 | 39.799 | 66.470 | −25.492 | 1.00 | 0.00 | C |
| ATOM | 7005 | CD | ARG | A 893 | 40.146 | 67.531 | −26.548 | 1.00 | 0.00 | C |
| ATOM | 7006 | NE | ARG | A 893 | 40.180 | 67.018 | −27.918 | 1.00 | 0.00 | N |
| ATOM | 7007 | CZ | ARG | A 893 | 41.096 | 66.179 | −28.394 | 1.00 | 0.00 | C |
| ATOM | 7008 | NH1 | ARG | A 893 | 42.078 | 65.738 | −27.615 | 1.00 | 0.00 | N |
| ATOM | 7009 | NH2 | ARG | A 893 | 41.039 | 65.788 | −29.660 | 1.00 | 0.00 | N |
| ATOM | 7010 | N | LEU | A 894 | 35.373 | 66.738 | −27.000 | 1.00 | 0.00 | N |
| ATOM | 7011 | CA | LEU | A 894 | 34.063 | 66.223 | −27.388 | 1.00 | 0.00 | C |
| ATOM | 7012 | C | LEU | A 894 | 34.253 | 65.245 | −28.541 | 1.00 | 0.00 | C |
| ATOM | 7013 | O | LEU | A 894 | 34.640 | 65.633 | −29.643 | 1.00 | 0.00 | O |
| ATOM | 7014 | CB | LEU | A 894 | 33.136 | 67.372 | −27.810 | 1.00 | 0.00 | C |
| ATOM | 7015 | CG | LEU | A 894 | 31.687 | 66.956 | −28.108 | 1.00 | 0.00 | C |
| ATOM | 7016 | CD1 | LEU | A 894 | 31.055 | 66.396 | −26.844 | 1.00 | 0.00 | C |
| ATOM | 7017 | CD2 | LEU | A 894 | 30.880 | 68.141 | −28.613 | 1.00 | 0.00 | C |
| ATOM | 7018 | N | VAL | A 895 | 33.975 | 63.972 | −28.276 | 1.00 | 0.00 | N |
| ATOM | 7019 | CA | VAL | A 895 | 34.163 | 62.927 | −29.272 | 1.00 | 0.00 | C |
| ATOM | 7020 | C | VAL | A 895 | 32.881 | 62.283 | −29.788 | 1.00 | 0.00 | C |
| ATOM | 7021 | O | VAL | A 895 | 32.201 | 61.573 | −29.048 | 1.00 | 0.00 | O |
| ATOM | 7022 | CB | VAL | A 895 | 35.044 | 61.788 | −28.708 | 1.00 | 0.00 | C |
| ATOM | 7023 | CG1 | VAL | A 895 | 35.391 | 60.801 | −29.817 | 1.00 | 0.00 | C |
| ATOM | 7024 | CG2 | VAL | A 895 | 36.300 | 62.361 | −28.070 | 1.00 | 0.00 | C |
| ATOM | 7025 | N | LEU | A 896 | 32.563 | 62.530 | −31.056 | 1.00 | 0.00 | N |
| ATOM | 7026 | CA | LEU | A 896 | 31.395 | 61.920 | −31.692 | 1.00 | 0.00 | C |
| ATOM | 7027 | C | LEU | A 896 | 31.964 | 60.752 | −32.498 | 1.00 | 0.00 | C |
| ATOM | 7028 | O | LEU | A 896 | 32.930 | 60.928 | −33.243 | 1.00 | 0.00 | O |
| ATOM | 7029 | CB | LEU | A 896 | 30.695 | 62.905 | −32.636 | 1.00 | 0.00 | C |
| ATOM | 7030 | CG | LEU | A 896 | 29.534 | 62.309 | −33.447 | 1.00 | 0.00 | C |
| ATOM | 7031 | CD1 | LEU | A 896 | 28.388 | 61.935 | −32.515 | 1.00 | 0.00 | C |
| ATOM | 7032 | CD2 | LEU | A 896 | 29.058 | 63.317 | −34.491 | 1.00 | 0.00 | C |
| ATOM | 7033 | N | GLU | A 897 | 31.380 | 59.567 | −32.343 | 1.00 | 0.00 | N |
| ATOM | 7034 | CA | GLU | A 897 | 31.878 | 58.384 | −33.044 | 1.00 | 0.00 | C |
| ATOM | 7035 | C | GLU | A 897 | 30.786 | 57.411 | −33.449 | 1.00 | 0.00 | C |
| ATOM | 7036 | O | GLU | A 897 | 29.726 | 57.362 | −32.830 | 1.00 | 0.00 | O |
| ATOM | 7037 | CB | GLU | A 897 | 32.833 | 57.596 | −32.148 | 1.00 | 0.00 | C |
| ATOM | 7038 | CG | GLU | A 897 | 34.008 | 58.349 | −31.593 | 1.00 | 0.00 | C |
| ATOM | 7039 | CD | GLU | A 897 | 34.773 | 57.507 | −30.587 | 1.00 | 0.00 | C |
| ATOM | 7040 | OE1 | GLU | A 897 | 34.193 | 57.167 | −29.534 | 1.00 | 0.00 | O |
| ATOM | 7041 | OE2 | GLU | A 897 | 35.944 | 57.179 | −30.855 | 1.00 | 0.00 | O |
| ATOM | 7042 | N | LYS | A 898 | 31.070 | 56.622 | −34.482 | 1.00 | 0.00 | N |
| ATOM | 7043 | CA | LYS | A 898 | 30.151 | 55.585 | −34.927 | 1.00 | 0.00 | C |
| ATOM | 7044 | C | LYS | A 898 | 30.618 | 54.362 | −34.132 | 1.00 | 0.00 | C |
| ATOM | 7045 | O | LYS | A 898 | 31.815 | 54.084 | −34.066 | 1.00 | 0.00 | O |
| ATOM | 7046 | CB | LYS | A 898 | 30.302 | 55.323 | −36.428 | 1.00 | 0.00 | C |
| ATOM | 7047 | CG | LYS | A 898 | 29.917 | 56.500 | −37.319 | 1.00 | 0.00 | C |
| ATOM | 7048 | CD | LYS | A 898 | 28.496 | 56.984 | −37.035 | 1.00 | 0.00 | C |
| ATOM | 7049 | CE | LYS | A 898 | 27.471 | 55.865 | −37.182 | 1.00 | 0.00 | C |
| ATOM | 7050 | NZ | LYS | A 898 | 26.095 | 56.347 | −36.886 | 1.00 | 0.00 | N |
| ATOM | 7051 | N | VAL | A 899 | 29.688 | 53.644 | −33.517 | 1.00 | 0.00 | N |
| ATOM | 7052 | CA | VAL | A 899 | 30.057 | 52.484 | −32.717 | 1.00 | 0.00 | C |
| ATOM | 7053 | C | VAL | A 899 | 29.325 | 51.202 | −33.115 | 1.00 | 0.00 | C |
| ATOM | 7054 | O | VAL | A 899 | 29.343 | 50.221 | −32.375 | 1.00 | 0.00 | O |
| ATOM | 7055 | CB | VAL | A 899 | 29.794 | 52.759 | −31.217 | 1.00 | 0.00 | C |
| ATOM | 7056 | CG1 | VAL | A 899 | 30.736 | 53.847 | −30.716 | 1.00 | 0.00 | C |
| ATOM | 7057 | CG2 | VAL | A 899 | 28.342 | 53.188 | −31.013 | 1.00 | 0.00 | C |
| ATOM | 7058 | N | ASN | A 900 | 28.691 | 51.204 | −34.283 | 1.00 | 0.00 | N |
| ATOM | 7059 | CA | ASN | A 900 | 27.955 | 50.024 | −34.728 | 1.00 | 0.00 | C |
| ATOM | 7060 | C | ASN | A 900 | 28.853 | 48.816 | −34.983 | 1.00 | 0.00 | C |
| ATOM | 7061 | O | ASN | A 900 | 28.384 | 47.679 | −34.962 | 1.00 | 0.00 | O |
| ATOM | 7062 | CB | ASN | A 900 | 27.146 | 50.342 | −35.993 | 1.00 | 0.00 | C |
| ATOM | 7063 | CG | ASN | A 900 | 27.997 | 50.926 | −37.095 | 1.00 | 0.00 | C |
| ATOM | 7064 | OD1 | ASN | A 900 | 28.598 | 51.988 | −36.931 | 1.00 | 0.00 | O |
| ATOM | 7065 | ND2 | ASN | A 900 | 28.056 | 50.235 | −38.230 | 1.00 | 0.00 | N |
| ATOM | 7066 | N | ASN | A 901 | 30.139 | 49.052 | −35.225 | 1.00 | 0.00 | N |
| ATOM | 7067 | CA | ASN | A 901 | 31.063 | 47.951 | −35.480 | 1.00 | 0.00 | C |
| ATOM | 7068 | C | ASN | A 901 | 31.850 | 47.538 | −34.244 | 1.00 | 0.00 | C |
| ATOM | 7069 | O | ASN | A 901 | 32.622 | 46.583 | −34.290 | 1.00 | 0.00 | O |
| ATOM | 7070 | CB | ASN | A 901 | 32.051 | 48.318 | −36.588 | 1.00 | 0.00 | C |
| ATOM | 7071 | CG | ASN | A 901 | 31.402 | 48.373 | −37.949 | 1.00 | 0.00 | C |
| ATOM | 7072 | OD1 | ASN | A 901 | 30.636 | 47.484 | −38.316 | 1.00 | 0.00 | O |
| ATOM | 7073 | ND2 | ASN | A 901 | 31.713 | 49.415 | −38.714 | 1.00 | 0.00 | N |
| ATOM | 7074 | N | CYS | A 902 | 31.657 | 48.253 | −33.143 | 1.00 | 0.00 | N |
| ATOM | 7075 | CA | CYS | A 902 | 32.378 | 47.943 | −31.915 | 1.00 | 0.00 | C |
| ATOM | 7076 | C | CYS | A 902 | 31.804 | 46.754 | −31.161 | 1.00 | 0.00 | C |
| ATOM | 7077 | O | CYS | A 902 | 30.588 | 46.583 | −31.080 | 1.00 | 0.00 | O |
| ATOM | 7078 | CB | CYS | A 902 | 32.371 | 49.139 | −30.965 | 1.00 | 0.00 | C |
| ATOM | 7079 | SG | CYS | A 902 | 33.115 | 50.688 | −31.564 | 1.00 | 0.00 | S |
| ATOM | 7080 | N | VAL | A 903 | 32.687 | 45.939 | −30.599 | 1.00 | 0.00 | N |
| ATOM | 7081 | CA | VAL | A 903 | 32.261 | 44.793 | −29.808 | 1.00 | 0.00 | C |
| ATOM | 7082 | C | VAL | A 903 | 31.992 | 45.343 | −28.411 | 1.00 | 0.00 | C |
| ATOM | 7083 | O | VAL | A 903 | 32.920 | 45.631 | −27.656 | 1.00 | 0.00 | O |
| ATOM | 7084 | CB | VAL | A 903 | 33.361 | 43.721 | −29.734 | 1.00 | 0.00 | C |
| ATOM | 7085 | CG1 | VAL | A 903 | 32.925 | 42.593 | −28.805 | 1.00 | 0.00 | C |
| ATOM | 7086 | CG2 | VAL | A 903 | 33.649 | 43.181 | −31.126 | 1.00 | 0.00 | C |
| ATOM | 7087 | N | ARG | A 904 | 30.718 | 45.506 | −28.079 | 1.00 | 0.00 | N |
| ATOM | 7088 | CA | ARG | A 904 | 30.335 | 46.051 | −26.784 | 1.00 | 0.00 | C |
| ATOM | 7089 | C | ARG | A 904 | 29.775 | 45.005 | −25.831 | 1.00 | 0.00 | C |
| ATOM | 7090 | O | ARG | A 904 | 29.446 | 43.890 | −26.239 | 1.00 | 0.00 | O |
| ATOM | 7091 | CB | ARG | A 904 | 29.306 | 47.169 | −26.986 | 1.00 | 0.00 | C |
| ATOM | 7092 | CG | ARG | A 904 | 29.892 | 48.416 | −27.629 | 1.00 | 0.00 | C |
| ATOM | 7093 | CD | ARG | A 904 | 28.821 | 49.447 | −27.952 | 1.00 | 0.00 | C |
| ATOM | 7094 | NE | ARG | A 904 | 28.025 | 49.073 | −29.118 | 1.00 | 0.00 | N |
| ATOM | 7095 | CZ | ARG | A 904 | 27.072 | 49.838 | −29.645 | 1.00 | 0.00 | C |
| ATOM | 7096 | NH1 | ARG | A 904 | 26.796 | 51.019 | −29.106 | 1.00 | 0.00 | N |
| ATOM | 7097 | NH2 | ARG | A 904 | 26.401 | 49.428 | −30.714 | 1.00 | 0.00 | N |
| ATOM | 7098 | N | PRO | A 905 | 29.678 | 45.350 | −24.537 | 1.00 | 0.00 | N |
| ATOM | 7099 | CA | PRO | A 905 | 29.145 | 44.423 | −23.535 | 1.00 | 0.00 | C |
| ATOM | 7100 | C | PRO | A 905 | 27.669 | 44.179 | −23.815 | 1.00 | 0.00 | C |
| ATOM | 7101 | O | PRO | A 905 | 27.011 | 45.009 | −24.450 | 1.00 | 0.00 | O |
| ATOM | 7102 | CB | PRO | A 905 | 29.353 | 45.172 | −22.223 | 1.00 | 0.00 | C |
| ATOM | 7103 | CG | PRO | A 905 | 30.529 | 46.057 | −22.510 | 1.00 | 0.00 | C |
| ATOM | 7104 | CD | PRO | A 905 | 30.220 | 46.559 | −23.891 | 1.00 | 0.00 | C |
| ATOM | 7105 | N | SER | A 906 | 27.148 | 43.049 | −23.344 | 1.00 | 0.00 | N |
| ATOM | 7106 | CA | SER | A 906 | 25.740 | 42.738 | −23.550 | 1.00 | 0.00 | C |
| ATOM | 7107 | C | SER | A 906 | 24.908 | 43.772 | −22.807 | 1.00 | 0.00 | C |
| ATOM | 7108 | O | SER | A 906 | 25.427 | 44.530 | −21.985 | 1.00 | 0.00 | O |
| ATOM | 7109 | CB | SER | A 906 | 25.399 | 41.348 | −23.014 | 1.00 | 0.00 | C |
| ATOM | 7110 | OG | SER | A 906 | 25.229 | 41.380 | −21.607 | 1.00 | 0.00 | O |
| ATOM | 7111 | N | LYS | A 907 | 23.612 | 43.783 | −23.092 | 1.00 | 0.00 | N |
| ATOM | 7112 | CA | LYS | A 907 | 22.691 | 44.723 | −22.469 | 1.00 | 0.00 | C |
| ATOM | 7113 | C | LYS | A 907 | 22.632 | 44.605 | −20.947 | 1.00 | 0.00 | C |
| ATOM | 7114 | O | LYS | A 907 | 22.245 | 45.550 | −20.265 | 1.00 | 0.00 | O |
| ATOM | 7115 | CB | LYS | A 907 | 21.289 | 44.526 | −23.056 | 1.00 | 0.00 | C |
| ATOM | 7116 | CG | LYS | A 907 | 21.188 | 44.870 | −24.538 | 1.00 | 0.00 | C |
| ATOM | 7117 | CD | LYS | A 907 | 19.871 | 44.382 | −25.152 | 1.00 | 0.00 | C |
| ATOM | 7118 | CE | LYS | A 907 | 18.652 | 44.923 | −24.409 | 1.00 | 0.00 | C |
| ATOM | 7119 | NZ | LYS | A 907 | 18.557 | 46.412 | −24.456 | 1.00 | 0.00 | N |
| ATOM | 7120 | N | LEU | A 908 | 23.025 | 43.453 | −20.414 | 1.00 | 0.00 | N |
| ATOM | 7121 | CA | LEU | A 908 | 22.976 | 43.233 | −18.971 | 1.00 | 0.00 | C |
| ATOM | 7122 | C | LEU | A 908 | 24.275 | 43.546 | −18.223 | 1.00 | 0.00 | C |
| ATOM | 7123 | O | LEU | A 908 | 24.312 | 43.504 | −16.994 | 1.00 | 0.00 | O |
| ATOM | 7124 | CB | LEU | A 908 | 22.549 | 41.789 | −18.683 | 1.00 | 0.00 | C |
| ATOM | 7125 | CG | LEU | A 908 | 21.173 | 41.378 | −19.229 | 1.00 | 0.00 | C |
| ATOM | 7126 | CD1 | LEU | A 908 | 20.900 | 39.920 | −18.895 | 1.00 | 0.00 | C |
| ATON | 7127 | CD2 | LEU | A 908 | 20.086 | 42.274 | −18.634 | 1.00 | 0.00 | C |
| ATOM | 7128 | N | HIS | A 909 | 25.336 | 43.857 | −18.959 | 1.00 | 0.00 | N |
| ATOM | 7129 | CA | HIS | A 909 | 26.623 | 44.172 | −18.343 | 1.00 | 0.00 | C |
| ATOM | 7130 | C | HIS | A 909 | 26.538 | 45.556 | −17.687 | 1.00 | 0.00 | C |
| ATOM | 7131 | O | HIS | A 909 | 26.028 | 46.501 | −18.287 | 1.00 | 0.00 | O |
| ATOM | 7132 | CB | HIS | A 909 | 27.723 | 44.148 | −19.408 | 1.00 | 0.00 | C |
| ATOM | 7133 | CG | HIS | A 909 | 29.098 | 43.932 | −18.859 | 1.00 | 0.00 | C |
| ATOM | 7134 | ND1 | HIS | A 909 | 29.772 | 44.889 | −18.130 | 1.00 | 0.00 | N |
| ATOM | 7135 | CD2 | HIS | A 909 | 29.927 | 42.863 | −18.933 | 1.00 | 0.00 | C |
| ATOM | 7136 | CE1 | HIS | A 909 | 30.956 | 44.420 | −17.781 | 1.00 | 0.00 | C |
| ATOM | 7137 | NE2 | HIS | A 909 | 31.076 | 43.193 | −18.257 | 1.00 | 0.00 | N |
| ATOM | 7138 | N | PRO | A 910 | 27.030 | 45.687 | −16.444 | 1.00 | 0.00 | N |
| ATOM | 7139 | CA | PRO | A 910 | 27.000 | 46.958 | −15.710 | 1.00 | 0.00 | C |
| ATOM | 7140 | C | PRO | A 910 | 28.073 | 47.968 | −16.111 | 1.00 | 0.00 | C |
| ATOM | 7141 | O | PRO | A 910 | 28.051 | 49.109 | −15.644 | 1.00 | 0.00 | O |
| ATOM | 7142 | CB | PRO | A 910 | 27.184 | 46.529 | −14.248 | 1.00 | 0.00 | C |
| ATOM | 7143 | CG | PRO | A 910 | 26.897 | 45.044 | −14.246 | 1.00 | 0.00 | C |
| ATOM | 7144 | CD | PRO | A 910 | 27.464 | 44.597 | −15.556 | 1.00 | 0.00 | C |
| ATOM | 7145 | N | ALA | A 911 | 29.013 | 47.557 | −16.956 | 1.00 | 0.00 | N |
| ATOM | 7146 | CA | ALA | A 911 | 30.093 | 48.449 | −17.358 | 1.00 | 0.00 | C |
| ATOM | 7147 | C | ALA | A 911 | 30.048 | 48.926 | −18.801 | 1.00 | 0.00 | C |
| ATOM | 7148 | O | ALA | A 911 | 29.282 | 48.424 | −19.624 | 1.00 | 0.00 | O |
| ATOM | 7149 | CB | ALA | A 911 | 31.442 | 47.775 | −17.091 | 1.00 | 0.00 | C |
| ATOM | 7150 | N | GLY | A 912 | 30.897 | 49.907 | −19.086 | 1.00 | 0.00 | N |
| ATOM | 7151 | CA | GLY | A 912 | 31.020 | 50.460 | −20.420 | 1.00 | 0.00 | C |
| ATOM | 7152 | C | GLY | A 912 | 32.492 | 50.754 | −20.617 | 1.00 | 0.00 | C |
| ATOM | 7153 | O | GLY | A 912 | 33.220 | 50.906 | −19.633 | 1.00 | 0.00 | O |
| ATOM | 7154 | N | TYR | A 913 | 32.941 | 50.828 | −21.868 | 1.00 | 0.00 | N |
| ATOM | 7155 | CA | TYR | A 913 | 34.346 | 51.103 | −22.150 | 1.00 | 0.00 | C |
| ATOM | 7156 | C | TYR | A 913 | 34.533 | 52.138 | −23.247 | 1.00 | 0.00 | C |
| ATOM | 7157 | O | TYR | A 913 | 33.729 | 52.233 | −24.173 | 1.00 | 0.00 | O |
| ATOM | 7158 | CB | TYR | A 913 | 35.074 | 49.812 | −22.542 | 1.00 | 0.00 | C |
| ATOM | 7159 | CG | TYR | A 913 | 35.025 | 48.761 | −21.459 | 1.00 | 0.00 | C |
| ATOM | 7160 | CD1 | TYR | A 913 | 34.031 | 47.786 | −21.453 | 1.00 | 0.00 | C |
| ATOM | 7161 | CD2 | TYR | A 913 | 35.931 | 48.786 | −20.400 | 1.00 | 0.00 | C |
| ATOM | 7162 | CE1 | TYR | A 913 | 33.934 | 46.864 | −20.415 | 1.00 | 0.00 | C |
| ATOM | 7163 | CE2 | TYR | A 913 | 35.844 | 47.869 | −19.356 | 1.00 | 0.00 | C |
| ATOM | 7164 | CZ | TYR | A 913 | 34.841 | 46.914 | −19.370 | 1.00 | 0.00 | C |
| ATOM | 7165 | OH | TYR | A 913 | 34.724 | 46.024 | −18.328 | 1.00 | 0.00 | O |
| ATOM | 7166 | N | LEU | A 914 | 35.608 | 52.911 | −23.132 | 1.00 | 0.00 | N |
| ATOM | 7167 | CA | LEU | A 914 | 35.930 | 53.941 | −24.112 | 1.00 | 0.00 | C |
| ATOM | 7168 | C | LEU | A 914 | 36.561 | 53.350 | −25.362 | 1.00 | 0.00 | C |
| ATOM | 7169 | O | LEU | A 914 | 37.007 | 52.196 | −25.378 | 1.00 | 0.00 | O |
| ATOM | 7170 | CB | LEU | A 914 | 36.916 | 54.960 | −23.525 | 1.00 | 0.00 | C |
| ATOM | 7171 | CG | LEU | A 914 | 36.485 | 55.854 | −22.357 | 1.00 | 0.00 | C |
| ATOM | 7172 | CD1 | LEU | A 914 | 37.599 | 56.849 | −22.060 | 1.00 | 0.00 | C |
| ATOM | 7173 | CD2 | LEU | A 914 | 35.190 | 56.592 | −22.701 | 1.00 | 0.00 | C |
| ATOM | 7174 | N | THR | A 915 | 36.587 | 54.167 | −26.409 | 1.00 | 0.00 | N |
| ATOM | 7175 | CA | THR | A 915 | 37.200 | 53.813 | −27.677 | 1.00 | 0.00 | C |
| ATOM | 7176 | C | THR | A 915 | 38.606 | 54.388 | −27.573 | 1.00 | 0.00 | C |
| ATOM | 7177 | O | THR | A 915 | 38.893 | 55.173 | −26.670 | 1.00 | 0.00 | O |
| ATOM | 7178 | CB | THR | A 915 | 36.514 | 54.519 | −28.852 | 1.00 | 0.00 | C |
| ATOM | 7179 | OG1 | THR | A 915 | 36.489 | 55.928 | −28.593 | 1.00 | 0.00 | O |
| ATOM | 7180 | CG2 | THR | A 915 | 35.097 | 54.011 | −29.047 | 1.00 | 0.00 | C |
| ATOM | 7181 | N | SER | A 916 | 39.474 | 54.007 | −28.501 | 1.00 | 0.00 | N |
| ATOM | 7182 | CA | SER | A 916 | 40.841 | 54.506 | −28.521 | 1.00 | 0.00 | C |
| ATOM | 7183 | C | SER | A 916 | 40.879 | 56.033 | −28.585 | 1.00 | 0.00 | C |
| ATOM | 7184 | O | SER | A 916 | 41.628 | 56.674 | −27.851 | 1.00 | 0.00 | O |
| ATOM | 7185 | CB | SER | A 916 | 41.584 | 53.936 | −29.728 | 1.00 | 0.00 | C |
| ATOM | 7186 | OG | SER | A 916 | 42.821 | 54.596 | −29.915 | 1.00 | 0.00 | O |
| ATOM | 7187 | N | ALA | A 917 | 40.065 | 56.613 | −29.463 | 1.00 | 0.00 | N |
| ATOM | 7188 | CA | ALA | A 917 | 40.035 | 58.067 | −29.619 | 1.00 | 0.00 | C |
| ATOM | 7189 | C | ALA | A 917 | 39.606 | 58.772 | −28.341 | 1.00 | 0.00 | C |
| ATOM | 7190 | O | ALA | A 917 | 40.203 | 59.777 | −27.948 | 1.00 | 0.00 | O |
| ATOM | 7191 | CB | ALA | A 917 | 39.100 | 58.454 | −30.759 | 1.00 | 0.00 | C |
| ATOM | 7192 | N | ALA | A 918 | 38.566 | 58.251 | −27.698 | 1.00 | 0.00 | N |
| ATOM | 7193 | CA | ALA | A 918 | 38.061 | 58.853 | −26.469 | 1.00 | 0.00 | C |
| ATOM | 7194 | C | ALA | A 918 | 39.092 | 58.749 | −25.349 | 1.00 | 0.00 | C |
| ATOM | 7195 | O | ALA | A 918 | 39.267 | 59.678 | −24.553 | 1.00 | 0.00 | O |
| ATOM | 7196 | CB | ALA | A 918 | 36.762 | 58.176 | −26.054 | 1.00 | 0.00 | C |
| ATOM | 7197 | N | HIS | A 919 | 39.774 | 57.613 | −25.286 | 1.00 | 0.00 | N |
| ATOM | 7198 | CA | HIS | A 919 | 40.788 | 57.409 | −24.261 | 1.00 | 0.00 | C |
| ATOM | 7199 | C | HIS | A 919 | 41.955 | 58.374 | −24.471 | 1.00 | 0.00 | C |
| ATOM | 7200 | O | HIS | A 919 | 42.406 | 59.031 | −23.530 | 1.00 | 0.00 | O |
| ATOM | 7201 | CB | HIS | A 919 | 41.282 | 55.958 | −24.285 | 1.00 | 0.00 | C |
| ATOM | 7202 | CG | HIS | A 919 | 42.389 | 55.682 | −23.315 | 1.00 | 0.00 | C |
| ATOM | 7203 | ND1 | HIS | A 919 | 43.662 | 55.340 | −23.718 | 1.00 | 0.00 | N |
| ATOM | 7204 | CD2 | HIS | A 919 | 42.422 | 55.729 | −21.963 | 1.00 | 0.00 | C |
| ATOM | 7205 | CE1 | HIS | A 919 | 44.433 | 55.190 | −22.656 | 1.00 | 0.00 | C |
| ATOM | 7206 | NE2 | HIS | A 919 | 43.705 | 55.421 | −21.578 | 1.00 | 0.00 | N |
| ATOM | 7207 | N | LYS | A 920 | 42.438 | 58.474 | −25.704 | 1.00 | 0.00 | N |
| ATOM | 7208 | CA | LYS | A 920 | 43.545 | 59.379 | −25.978 | 1.00 | 0.00 | C |
| ATOM | 7209 | C | LYS | A 920 | 43.142 | 60.825 | −25.712 | 1.00 | 0.00 | C |
| ATOM | 7210 | O | LYS | A 920 | 43.961 | 61.627 | −25.257 | 1.00 | 0.00 | O |
| ATOM | 7211 | CB | LYS | A 920 | 44.041 | 59.215 | −27.420 | 1.00 | 0.00 | C |
| ATOM | 7212 | CG | LYS | A 920 | 44.916 | 57.974 | −27.614 | 1.00 | 0.00 | C |
| ATOM | 7213 | CD | LYS | A 920 | 45.588 | 57.952 | −28.980 | 1.00 | 0.00 | C |
| ATOM | 7214 | CE | LYS | A 920 | 46.543 | 56.779 | −29.094 | 1.00 | 0.00 | C |
| ATOM | 7215 | NZ | LYS | A 920 | 47.662 | 56.877 | −28.112 | 1.00 | 0.00 | N |
| ATOM | 7216 | N | ALA | A 921 | 41.882 | 61.153 | −25.985 | 1.00 | 0.00 | N |
| ATOM | 7217 | CA | ALA | A 921 | 41.392 | 62.511 | −25.755 | 1.00 | 0.00 | C |
| ATOM | 7218 | C | ALA | A 921 | 41.437 | 62.810 | −24.254 | 1.00 | 0.00 | C |
| ATOM | 7219 | O | ALA | A 921 | 41.793 | 63.915 | −23.840 | 1.00 | 0.00 | O |
| ATOM | 7220 | CB | ALA | A 921 | 39.964 | 62.658 | −26.295 | 1.00 | 0.00 | C |
| ATOM | 7221 | N | SER | A 922 | 41.084 | 61.823 | −23.435 | 1.00 | 0.00 | N |
| ATOM | 7222 | CA | SER | A 922 | 41.119 | 62.014 | −21.989 | 1.00 | 0.00 | C |
| ATOM | 7223 | C | SER | A 922 | 42.558 | 62.256 | −21.541 | 1.00 | 0.00 | C |
| ATOM | 7224 | O | SER | A 922 | 42.820 | 63.112 | −20.700 | 1.00 | 0.00 | O |
| ATOM | 7225 | CB | SER | A 922 | 40.571 | 60.783 | −21.261 | 1.00 | 0.00 | C |
| ATOM | 7226 | OG | SER | A 922 | 40.656 | 60.960 | −19.853 | 1.00 | 0.00 | O |
| ATOM | 7227 | N | GLN | A 923 | 43.492 | 61.499 | −22.108 | 1.00 | 0.00 | N |
| ATOM | 7228 | CA | GLN | A 923 | 44.896 | 61.650 | −21.748 | 1.00 | 0.00 | C |
| ATOM | 7229 | C | GLN | A 923 | 45.434 | 63.022 | −22.152 | 1.00 | 0.00 | C |
| ATOM | 7230 | O | GLN | A 923 | 46.315 | 63.565 | −21.483 | 1.00 | 0.00 | O |
| ATOM | 7231 | CB | GLN | A 923 | 45.739 | 60.551 | −22.398 | 1.00 | 0.00 | C |
| ATOM | 7232 | CG | GLN | A 923 | 45.409 | 59.138 | −21.921 | 1.00 | 0.00 | C |
| ATOM | 7233 | CD | GLN | A 923 | 46.343 | 58.104 | −22.517 | 1.00 | 0.00 | C |
| ATOM | 7234 | OE1 | GLN | A 923 | 46.494 | 58.027 | −23.737 | 1.00 | 0.00 | O |
| ATOM | 7235 | NE2 | GLN | A 923 | 46.980 | 57.301 | −21.658 | 1.00 | 0.00 | N |
| ATOM | 7236 | N | SER | A 924 | 44.901 | 63.582 | −23.236 | 1.00 | 0.00 | N |
| ATOM | 7237 | CA | SER | A 924 | 45.344 | 64.895 | −23.703 | 1.00 | 0.00 | C |
| ATOM | 7238 | C | SER | A 924 | 44.952 | 65.979 | −22.703 | 1.00 | 0.00 | C |
| ATOM | 7239 | O | SER | A 924 | 45.596 | 67.026 | −22.628 | 1.00 | 0.00 | O |
| ATOM | 7240 | CB | SER | A 924 | 44.728 | 65.227 | −25.067 | 1.00 | 0.00 | C |
| ATOM | 7241 | OG | SER | A 924 | 43.364 | 65.602 | −24.936 | 1.00 | 0.00 | O |
| ATOM | 7242 | N | LEU | A 925 | 43.889 | 65.728 | −21.944 | 1.00 | 0.00 | N |
| ATOM | 7243 | CA | LEU | A 925 | 43.413 | 66.686 | −20.951 | 1.00 | 0.00 | C |
| ATOM | 7244 | C | LEU | A 925 | 44.095 | 66.525 | −19.592 | 1.00 | 0.00 | C |
| ATOM | 7245 | O | LEU | A 925 | 44.482 | 67.509 | −18.959 | 1.00 | 0.00 | O |
| ATOM | 7246 | CB | LEU | A 925 | 41.897 | 66.535 | −20.761 | 1.00 | 0.00 | C |
| ATOM | 7247 | CG | LEU | A 925 | 41.003 | 66.794 | −21.978 | 1.00 | 0.00 | C |
| ATOM | 7248 | CD1 | LEU | A 925 | 39.558 | 66.416 | −21.647 | 1.00 | 0.00 | C |
| ATOM | 7249 | CD2 | LEU | A 925 | 41.097 | 68.256 | −22.385 | 1.00 | 0.00 | C |
| ATOM | 7250 | N | LEU | A 926 | 44.242 | 65.284 | −19.146 | 1.00 | 0.00 | N |
| ATOM | 7251 | CA | LEU | A 926 | 44.838 | 65.026 | −17.840 | 1.00 | 0.00 | C |
| ATOM | 7252 | C | LEU | A 926 | 46.357 | 64.965 | −17.804 | 1.00 | 0.00 | C |
| ATOM | 7253 | O | LEU | A 926 | 46.962 | 65.310 | −16.790 | 1.00 | 0.00 | O |
| ATOM | 7254 | CB | LEU | A 926 | 44.270 | 63.730 | −17.252 | 1.00 | 0.00 | C |
| ATOM | 7255 | CG | LEU | A 926 | 42.757 | 63.721 | −17.007 | 1.00 | 0.00 | C |
| ATOM | 7256 | CD1 | LEU | A 926 | 42.344 | 62.384 | −16.408 | 1.00 | 0.00 | C |
| ATOM | 7257 | CD2 | LEU | A 926 | 42.376 | 64.868 | −16.072 | 1.00 | 0.00 | C |
| ATOM | 7258 | N | ASP | A 927 | 46.976 | 64.522 | −18.892 | 1.00 | 0.00 | N |
| ATOM | 7259 | CA | ASP | A 927 | 43.431 | 64.416 | −18.923 | 1.00 | 0.00 | C |
| ATOM | 7260 | C | ASP | A 927 | 49.021 | 64.910 | −20.233 | 1.00 | 0.00 | C |
| ATOM | 7261 | O | ASP | A 927 | 49.584 | 64.140 | −21.009 | 1.00 | 0.00 | O |
| ATOM | 7262 | CB | ASP | A 927 | 48.846 | 62.968 | −18.665 | 1.00 | 0.00 | C |
| ATOM | 7263 | CG | ASP | A 927 | 48.501 | 62.517 | −17.259 | 1.00 | 0.00 | C |
| ATOM | 7264 | OD1 | ASP | A 927 | 49.213 | 62.918 | −16.315 | 1.00 | 0.00 | O |
| ATOM | 7265 | OD2 | ASP | A 927 | 47.510 | 61.779 | −17.097 | 1.00 | 0.00 | O |
| ATOM | 7266 | N | PRO | A 928 | 48.901 | 66.221 | −20.488 | 1.00 | 0.00 | N |
| ATOM | 7267 | CA | PRO | A 928 | 49.419 | 66.837 | −21.709 | 1.00 | 0.00 | C |
| ATOM | 7268 | C | PRO | A 928 | 50.933 | 66.933 | −21.660 | 1.00 | 0.00 | C |
| ATOM | 7269 | O | PRO | A 928 | 51.550 | 66.675 | −20.622 | 1.00 | 0.00 | O |
| ATOM | 7270 | CB | PRO | A 928 | 48.775 | 68.217 | −21.686 | 1.00 | 0.00 | C |
| ATOM | 7271 | CG | PRO | A 928 | 48.824 | 68.553 | −20.231 | 1.00 | 0.00 | C |
| ATOM | 7272 | CD | PRO | A 928 | 48.356 | 67.247 | −19.579 | 1.00 | 0.00 | C |
| ATOM | 7273 | N | LEU | A 929 | 51.531 | 67.302 | −22.786 | 1.00 | 0.00 | N |
| ATOM | 7274 | CA | LEU | A 929 | 52.972 | 67.476 | −22.827 | 1.00 | 0.00 | C |
| ATOM | 7275 | C | LEU | A 929 | 53.279 | 68.648 | −21.902 | 1.00 | 0.00 | C |
| ATOM | 7276 | O | LEU | A 929 | 52.476 | 69.571 | −21.777 | 1.00 | 0.00 | O |
| ATOM | 7277 | CB | LEU | A 929 | 53.437 | 67.849 | −24.236 | 1.00 | 0.00 | C |
| ATOM | 7278 | CG | LEU | A 929 | 53.238 | 66.866 | −25.385 | 1.00 | 0.00 | C |
| ATOM | 7279 | CD1 | LEU | A 929 | 53.804 | 67.483 | −26.665 | 1.00 | 0.00 | C |
| ATOM | 7280 | CD2 | LEU | A 929 | 53.945 | 65.551 | −25.074 | 1.00 | 0.00 | C |
| ATOM | 7281 | N | ASP | A 930 | 54.431 | 68.601 | −21.244 | 1.00 | 0.00 | N |
| ATOM | 7282 | CA | ASP | A 930 | 54.851 | 69.695 | −20.379 | 1.00 | 0.00 | C |
| ATOM | 7283 | C | ASP | A 930 | 55.776 | 70.560 | −21.226 | 1.00 | 0.00 | C |
| ATOM | 7284 | O | ASP | A 930 | 56.530 | 70.040 | −22.051 | 1.00 | 0.00 | O |
| ATOM | 7285 | CB | ASP | A 930 | 55.595 | 69.151 | −19.167 | 1.00 | 0.00 | C |
| ATOM | 7286 | CG | ASP | A 930 | 54.765 | 68.166 | −18.392 | 1.00 | 0.00 | C |
| ATOM | 7287 | OD1 | ASP | A 930 | 53.736 | 68.594 | −17.828 | 1.00 | 0.00 | O |
| ATOM | 7288 | OD2 | ASP | A 930 | 55.130 | 66.972 | −18.362 | 1.00 | 0.00 | O |
| ATOM | 7289 | N | LYS | A 931 | 55.721 | 71.871 | −21.024 | 1.00 | 0.00 | N |
| ATOM | 7290 | CA | LYS | A 931 | 56.540 | 72.791 | −21.807 | 1.00 | 0.00 | C |
| ATOM | 7291 | C | LYS | A 931 | 57.511 | 73.605 | −20.966 | 1.00 | 0.00 | C |
| ATOM | 7292 | O | LYS | A 931 | 57.118 | 74.237 | −19.985 | 1.00 | 0.00 | O |
| ATOM | 7293 | CB | LYS | A 931 | 55.628 | 73.735 | −22.601 | 1.00 | 0.00 | C |
| ATOM | 7294 | CG | LYS | A 931 | 54.629 | 73.017 | −23.506 | 1.00 | 0.00 | C |
| ATOM | 7295 | CD | LYS | A 931 | 53.721 | 74.004 | −24.239 | 1.00 | 0.00 | C |
| ATOM | 7296 | CE | LYS | A 931 | 52.860 | 74.794 | −23.266 | 1.00 | 0.00 | C |
| ATOM | 7297 | NZ | LYS | A 931 | 51.982 | 75.779 | −23.948 | 1.00 | 0.00 | N |
| ATOM | 7298 | N | PHE | A 932 | 58.779 | 73.605 | −21.374 | 1.00 | 0.00 | N |
| ATOM | 7299 | CA | PHE | A 932 | 59.824 | 74.335 | −20.663 | 1.00 | 0.00 | C |
| ATOM | 7300 | C | PHE | A 932 | 60.511 | 75.376 | −21.548 | 1.00 | 0.00 | C |
| ATOM | 7301 | O | PHE | A 932 | 60.877 | 75.088 | −22.682 | 1.00 | 0.00 | O |
| ATOM | 7302 | CB | PHE | A 932 | 60.893 | 73.369 | −20.141 | 1.00 | 0.00 | C |
| ATOM | 7303 | CG | PHE | A 932 | 60.359 | 72.274 | −19.265 | 1.00 | 0.00 | C |
| ATOM | 7304 | CD1 | PHE | A 932 | 59.740 | 71.158 | −19.820 | 1.00 | 0.00 | C |
| ATOM | 7305 | CD2 | PHE | A 932 | 60.498 | 72.346 | −17.882 | 1.00 | 0.00 | C |
| ATOM | 7306 | CE1 | PHE | A 932 | 59.269 | 70.123 | −19.009 | 1.00 | 0.00 | C |
| ATOM | 7307 | CE2 | PHE | A 932 | 60.033 | 71.321 | −17.062 | 1.00 | 0.00 | C |
| ATOM | 7308 | CZ | PHE | A 932 | 59.417 | 70.206 | −17.627 | 1.00 | 0.00 | C |
| ATOM | 7309 | N | ILE | A 933 | 60.677 | 76.586 | −21.020 | 1.00 | 0.00 | N |
| ATOM | 7310 | CA | ILE | A 933 | 61.345 | 77.666 | −21.750 | 1.00 | 0.00 | C |
| ATOM | 7311 | C | ILE | A 933 | 62.697 | 77.920 | −21.083 | 1.00 | 0.00 | C |
| ATOM | 7312 | O | ILE | A 933 | 62.750 | 78.202 | −19.887 | 1.00 | 0.00 | O |
| ATOM | 7313 | CB | ILE | A 933 | 60.540 | 78.982 | −21.685 | 1.00 | 0.00 | C |
| ATOM | 7314 | CG1 | ILE | A 933 | 59.119 | 78.764 | −22.208 | 1.00 | 0.00 | C |
| ATOM | 7315 | CG2 | ILE | A 933 | 61.262 | 80.066 | −22.483 | 1.00 | 0.00 | C |
| ATOM | 7316 | CD1 | ILE | A 933 | 58.211 | 79.978 | −22.038 | 1.00 | 0.00 | C |
| ATOM | 7317 | N | PHE | A 934 | 63.788 | 77.817 | −21.841 | 1.00 | 0.00 | N |
| ATOM | 7318 | CA | PHE | A 934 | 65.109 | 78.048 | −21.264 | 1.00 | 0.00 | C |
| ATOM | 7319 | C | PHE | A 934 | 65.204 | 79.506 | −20.807 | 1.00 | 0.00 | C |
| ATOM | 7320 | O | PHE | A 934 | 64.931 | 80.424 | −21.578 | 1.00 | 0.00 | O |
| ATOM | 7321 | CB | PHE | A 934 | 66.206 | 77.745 | −22.286 | 1.00 | 0.00 | C |
| ATOM | 7322 | CG | PHE | A 934 | 67.583 | 77.727 | −21.692 | 1.00 | 0.00 | C |
| ATOM | 7323 | CD1 | PHE | A 934 | 67.965 | 76.704 | −20.831 | 1.00 | 0.00 | C |
| ATOM | 7324 | CD2 | PHE | A 934 | 68.480 | 78.758 | −21.953 | 1.00 | 0.00 | C |
| ATOM | 7325 | CE1 | PHE | A 934 | 69.225 | 76.704 | −20.230 | 1.00 | 0.00 | C |
| ATOM | 7326 | CE2 | PHE | A 934 | 69.741 | 78.772 | −21.362 | 1.00 | 0.00 | C |
| ATOM | 7327 | CZ | PHE | A 934 | 70.115 | 77.743 | −20.496 | 1.00 | 0.00 | C |
| ATOM | 7328 | N | ALA | A 935 | 65.599 | 79.713 | −19.552 | 1.00 | 0.00 | N |
| ATOM | 7329 | CA | ALA | A 935 | 65.685 | 81.055 | −18.974 | 1.00 | 0.00 | C |
| ATOM | 7330 | C | ALA | A 935 | 66.829 | 81.945 | −19.461 | 1.00 | 0.00 | C |
| ATOM | 7331 | O | ALA | A 935 | 66.609 | 83.111 | −19.799 | 1.00 | 0.00 | O |
| ATOM | 7332 | CB | ALA | A 935 | 65.736 | 80.952 | −17.455 | 1.00 | 0.00 | C |
| ATOM | 7333 | N | GLU | A 936 | 68.043 | 81.404 | −19.490 | 1.00 | 0.00 | N |
| ATOM | 7334 | CA | GLU | A 936 | 69.217 | 82.164 | −19.916 | 1.00 | 0.00 | C |
| ATOM | 7335 | C | GLU | A 936 | 69.257 | 82.388 | −21.426 | 1.00 | 0.00 | C |
| ATOM | 7336 | O | GLU | A 936 | 68.447 | 81.833 | −22.168 | 1.00 | 0.00 | O |
| ATOM | 7337 | CB | GLU | A 936 | 70.493 | 81.442 | −19.474 | 1.00 | 0.00 | C |
| ATOM | 7338 | CG | GLU | A 936 | 70.652 | 81.301 | −17.966 | 1.00 | 0.00 | C |
| ATOM | 7339 | CD | GLU | A 936 | 71.755 | 80.322 | −17.587 | 1.00 | 0.00 | C |
| ATOM | 7340 | OE1 | GLU | A 936 | 71.554 | 79.099 | −17.755 | 1.00 | 0.00 | O |
| ATOM | 7341 | OE2 | GLU | A 936 | 72.827 | 80.774 | −17.132 | 1.00 | 0.00 | O |
| ATOM | 7342 | N | ASN | A 937 | 70.206 | 83.200 | −21.879 | 1.00 | 0.00 | N |
| ATOM | 7343 | CA | ASN | A 937 | 70.320 | 83.484 | −23.302 | 1.00 | 0.00 | C |
| ATOM | 7344 | C | ASN | A 937 | 70.888 | 82.316 | −24.093 | 1.00 | 0.00 | C |
| ATOM | 7345 | O | ASN | A 937 | 70.475 | 82.079 | −25.228 | 1.00 | 0.00 | O |
| ATOM | 7346 | CB | ASN | A 937 | 71.175 | 84.734 | −23.537 | 1.00 | 0.00 | C |
| ATOM | 7347 | CG | ASN | A 937 | 70.507 | 85.997 | −23.030 | 1.00 | 0.00 | C |
| ATOM | 7348 | OD1 | ASN | A 937 | 69.289 | 86.157 | −23.136 | 1.00 | 0.00 | O |
| ATOM | 7349 | ND2 | ASN | A 937 | 71.303 | 86.909 | −22.491 | 1.00 | 0.00 | N |
| ATOM | 7350 | N | GLU | A 938 | 71.830 | 81.586 | −23.501 | 1.00 | 0.00 | N |
| ATOM | 7351 | CA | GLU | A 938 | 72.429 | 80.444 | −24.185 | 1.00 | 0.00 | C |
| ATOM | 7352 | C | GLU | A 938 | 72.505 | 79.186 | −23.324 | 1.00 | 0.00 | C |
| ATOM | 7353 | O | GLU | A 938 | 72.896 | 79.234 | −22.157 | 1.00 | 0.00 | O |
| ATOM | 7354 | CB | GLU | A 938 | 73.830 | 80.797 | −24.697 | 1.00 | 0.00 | C |
| ATOM | 7355 | CG | GLU | A 938 | 74.579 | 79.600 | −25.272 | 1.00 | 0.00 | C |
| ATOM | 7356 | CD | GLU | A 938 | 75.803 | 79.992 | −26.073 | 1.00 | 0.00 | C |
| ATOM | 7357 | OE1 | GLU | A 938 | 76.643 | 80.756 | −25.551 | 1.00 | 0.00 | O |
| ATOM | 7358 | OE2 | GLU | A 938 | 75.926 | 79.527 | −27.227 | 1.00 | 0.00 | O |
| ATOM | 7359 | N | TRP | A 939 | 72.128 | 78.060 | −23.921 | 1.00 | 0.00 | N |
| ATOM | 7360 | CA | TRP | A 939 | 72.147 | 76.771 | −23.240 | 1.00 | 0.00 | C |
| ATOM | 7361 | C | TRP | A 939 | 73.332 | 75.968 | −23.767 | 1.00 | 0.00 | C |
| ATOM | 7362 | O | TRP | A 939 | 73.240 | 75.302 | −24.796 | 1.00 | 0.00 | O |
| ATOM | 7363 | CB | TRP | A 939 | 70.830 | 76.029 | −23.506 | 1.00 | 0.00 | C |
| ATOM | 7364 | CG | TRP | A 939 | 70.711 | 74.676 | −22.854 | 1.00 | 0.00 | C |
| ATOM | 7365 | CD1 | TRP | A 939 | 71.603 | 74.083 | −22.004 | 1.00 | 0.00 | C |
| ATOM | 7366 | CD2 | TRP | A 939 | 69.631 | 73.747 | −23.012 | 1.00 | 0.00 | C |
| ATOM | 7367 | NE1 | TRP | A 939 | 71.144 | 72.842 | −21.624 | 1.00 | 0.00 | N |
| ATOM | 7368 | CE2 | TRP | A 939 | 69.936 | 72.612 | −22.229 | 1.00 | 0.00 | C |
| ATOM | 7369 | CE3 | TRP | A 939 | 68.435 | 73.764 | −23.742 | 1.00 | 0.00 | C |
| ATOM | 7370 | CZ2 | TRP | A 939 | 69.085 | 71.503 | −22.154 | 1.00 | 0.00 | C |
| ATOM | 7371 | CZ3 | TRP | A 939 | 67.588 | 72.661 | −23.668 | 1.00 | 0.00 | C |
| ATOM | 7372 | CH2 | TRP | A 939 | 67.920 | 71.547 | −22.878 | 1.00 | 0.00 | C |
| ATOM | 7373 | N | ILE | A 940 | 74.452 | 76.048 | −23.059 | 1.00 | 0.00 | N |
| ATOM | 7374 | CA | ILE | A 940 | 75.659 | 75.337 | −23.466 | 1.00 | 0.00 | C |
| ATOM | 7375 | C | ILE | A 940 | 75.550 | 73.837 | −23.190 | 1.00 | 0.00 | C |
| ATOM | 7376 | O | ILE | A 940 | 75.172 | 73.425 | −22.095 | 1.00 | 0.00 | O |
| ATOM | 7377 | CB | ILE | A 940 | 76.897 | 75.912 | −22.741 | 1.00 | 0.00 | C |
| ATOM | 7378 | CG1 | ILE | A 940 | 77.062 | 77.390 | −23.108 | 1.00 | 0.00 | C |
| ATOM | 7379 | CG2 | ILE | A 940 | 78.144 | 75.135 | −23.127 | 1.00 | 0.00 | C |
| ATOM | 7380 | CD1 | ILE | A 940 | 78.216 | 78.080 | −22.406 | 1.00 | 0.00 | C |
| ATOM | 7381 | N | GLY | A 941 | 75.872 | 73.029 | −24.194 | 1.00 | 0.00 | N |
| ATOM | 7382 | CA | GLY | A 941 | 75.808 | 71.586 | −24.035 | 1.00 | 0.00 | C |
| ATOM | 7383 | C | GLY | A 941 | 74.421 | 71.000 | −24.241 | 1.00 | 0.00 | C |
| ATOM | 7384 | O | GLY | A 941 | 74.192 | 69.823 | −23.957 | 1.00 | 0.00 | O |
| ATOM | 7385 | N | ALA | A 942 | 73.500 | 71.815 | −24.749 | 1.00 | 0.00 | N |
| ATOM | 7386 | CA | ALA | A 942 | 72.129 | 71.375 | −24.993 | 1.00 | 0.00 | C |
| ATOM | 7387 | C | ALA | A 942 | 72.022 | 70.166 | −25.923 | 1.00 | 0.00 | C |
| ATOM | 7388 | O | ALA | A 942 | 72.756 | 70.057 | −26.907 | 1.00 | 0.00 | O |
| ATOM | 7389 | CB | ALA | A 942 | 71.310 | 72.532 | −25.561 | 1.00 | 0.00 | C |
| ATOM | 7390 | N | GLN | A 943 | 71.098 | 69.264 | −25.603 | 1.00 | 0.00 | N |
| ATOM | 7391 | CA | GLN | A 943 | 70.862 | 68.070 | −26.410 | 1.00 | 0.00 | C |
| ATOM | 7392 | C | GLN | A 943 | 69.431 | 68.132 | −26.938 | 1.00 | 0.00 | C |
| ATOM | 7393 | O | GLN | A 943 | 68.564 | 68.759 | −26.328 | 1.00 | 0.00 | O |
| ATOM | 7394 | CB | GLN | A 943 | 71.081 | 66.807 | −25.573 | 1.00 | 0.00 | C |
| ATOM | 7395 | CG | GLN | A 943 | 72.451 | 66.766 | −24.909 | 1.00 | 0.00 | C |
| ATOM | 7396 | CD | GLN | A 943 | 72.715 | 65.465 | −24.176 | 1.00 | 0.00 | C |
| ATOM | 7397 | OE1 | GLN | A 943 | 71.863 | 64.974 | −23.432 | 1.00 | 0.00 | O |
| ATOM | 7398 | NE2 | GLN | A 943 | 73.906 | 64.905 | −24.372 | 1.00 | 0.00 | N |
| ATOM | 7399 | N | GLY | A 944 | 69.178 | 67.475 | −28.064 | 1.00 | 0.00 | N |
| ATOM | 7400 | CA | GLY | A 944 | 67.857 | 67.544 | −28.658 | 1.00 | 0.00 | C |
| ATOM | 7401 | C | GLY | A 944 | 66.794 | 66.553 | −28.247 | 1.00 | 0.00 | C |
| ATOM | 7402 | O | GLY | A 944 | 65.619 | 66.765 | −28.540 | 1.00 | 0.00 | O |
| ATOM | 7403 | N | GLN | A 945 | 67.175 | 65.480 | −27.565 | 1.00 | 0.00 | N |
| ATOM | 7404 | CA | GLN | A 945 | 66.180 | 64.488 | −27.191 | 1.00 | 0.00 | C |
| ATOM | 7405 | C | GLN | A 945 | 66.628 | 63.536 | −26.090 | 1.00 | 0.00 | C |
| ATOM | 7406 | O | GLN | A 945 | 67.820 | 63.280 | −25.914 | 1.00 | 0.00 | O |
| ATOM | 7407 | CB | GLN | A 945 | 65.800 | 63.694 | −28.444 | 1.00 | 0.00 | C |
| ATOM | 7408 | CG | GLN | A 945 | 64.874 | 62.512 | −28.238 | 1.00 | 0.00 | C |
| ATOM | 7409 | CD | GLN | A 945 | 64.618 | 61.765 | −29.537 | 1.00 | 0.00 | C |
| ATOM | 7410 | OE1 | GLN | A 945 | 63.877 | 62.233 | −30.406 | 1.00 | 0.00 | O |
| ATOM | 7411 | NE2 | GLN | A 945 | 65.244 | 60.606 | −29.682 | 1.00 | 0.00 | N |
| ATOM | 7412 | N | PHE | A 946 | 65.649 | 63.026 | −25.348 | 1.00 | 0.00 | N |
| ATOM | 7413 | CA | PHE | A 946 | 65.886 | 62.060 | −24.286 | 1.00 | 0.00 | C |
| ATOM | 7414 | C | PHE | A 946 | 64.790 | 61.007 | −24.344 | 1.00 | 0.00 | C |
| ATOM | 7415 | O | PHE | A 946 | 63.610 | 61.339 | −24.468 | 1.00 | 0.00 | O |
| ATOM | 7416 | CB | PHE | A 946 | 65.864 | 62.720 | −22.904 | 1.00 | 0.00 | C |
| ATOM | 7417 | CG | PHE | A 946 | 65.751 | 61.731 | −21.773 | 1.00 | 0.00 | C |
| ATOM | 7418 | CD1 | PHE | A 946 | 66.778 | 60.828 | −21.516 | 1.00 | 0.00 | C |
| ATOM | 7419 | CD2 | PHE | A 946 | 64.588 | 61.658 | −21.009 | 1.00 | 0.00 | C |
| ATOM | 7420 | CE1 | PHE | A 946 | 66.652 | 59.854 | −20.514 | 1.00 | 0.00 | C |
| ATOM | 7421 | CE2 | PHE | A 946 | 64.447 | 60.691 | −20.005 | 1.00 | 0.00 | C |
| ATOM | 7422 | CZ | PHE | A 946 | 65.482 | 59.784 | −19.758 | 1.00 | 0.00 | C |
| ATOM | 7423 | N | GLY | A 947 | 65.185 | 59.741 | −24.260 | 1.00 | 0.00 | N |
| ATOM | 7424 | CA | GLY | A 947 | 64.222 | 58.657 | −24.279 | 1.00 | 0.00 | C |
| ATOM | 7425 | C | GLY | A 947 | 63.867 | 58.092 | −25.638 | 1.00 | 0.00 | C |
| ATOM | 7426 | O | GLY | A 947 | 62.903 | 57.339 | −25.758 | 1.00 | 0.00 | O |
| ATOM | 7427 | N | GLY | A 948 | 64.632 | 58.447 | −26.665 | 1.00 | 0.00 | N |
| ATOM | 7428 | CA | GLY | A 948 | 64.347 | 57.929 | −27.989 | 1.00 | 0.00 | C |
| ATOM | 7429 | C | GLY | A 948 | 64.381 | 56.413 | −28.006 | 1.00 | 0.00 | C |
| ATOM | 7430 | O | GLY | A 948 | 63.780 | 55.779 | −28.872 | 1.00 | 0.00 | O |
| ATOM | 7431 | N | ASP | A 949 | 65.078 | 55.829 | −27.037 | 1.00 | 0.00 | N |
| ATOM | 7432 | CA | ASP | A 949 | 65.195 | 54.379 | −26.946 | 1.00 | 0.00 | C |
| ATOM | 7433 | C | ASP | A 949 | 64.172 | 53.754 | −25.998 | 1.00 | 0.00 | C |
| ATOM | 7434 | O | ASP | A 949 | 64.158 | 52.535 | −25.816 | 1.00 | 0.00 | O |
| ATOM | 7435 | CB | ASP | A 949 | 66.615 | 53.999 | −26.507 | 1.00 | 0.00 | C |
| ATOM | 7436 | CG | ASP | A 949 | 66.936 | 54.467 | −25.100 | 1.00 | 0.00 | C |
| ATOM | 7437 | OD1 | ASP | A 949 | 66.490 | 55.571 | −24.717 | 1.00 | 0.00 | O |
| ATOM | 7438 | OD2 | ASP | A 949 | 67.646 | 53.736 | −24.379 | 1.00 | 0.00 | O |
| ATOM | 7439 | N | HIS | A 950 | 63.321 | 54.578 | −25.388 | 1.00 | 0.00 | N |
| ATOM | 7440 | CA | HIS | A 950 | 62.299 | 54.054 | −24.482 | 1.00 | 0.00 | C |
| ATOM | 7441 | C | HIS | A 950 | 61.272 | 53.277 | −25.297 | 1.00 | 0.00 | C |
| ATOM | 7442 | O | HIS | A 950 | 60.897 | 53.696 | −26.388 | 1.00 | 0.00 | O |
| ATOM | 7443 | CB | HIS | A 950 | 61.562 | 55.180 | −23.750 | 1.00 | 0.00 | C |
| ATOM | 7444 | CG | HIS | A 950 | 62.387 | 55.898 | −22.728 | 1.00 | 0.00 | C |
| ATOM | 7445 | ND1 | HIS | A 950 | 63.627 | 55.460 | −22.320 | 1.00 | 0.00 | N |
| ATOM | 7446 | CD2 | HIS | A 950 | 62.131 | 57.020 | −22.015 | 1.00 | 0.00 | C |
| ATOM | 7447 | CE1 | HIS | A 950 | 64.101 | 56.282 | −21.400 | 1.00 | 0.00 | C |
| ATOM | 7448 | NE2 | HIS | A 950 | 63.211 | 57.237 | −21.197 | 1.00 | 0.00 | N |
| ATOM | 7449 | N | PRO | A 951 | 60.806 | 52.132 | −24.779 | 1.00 | 0.00 | N |
| ATOM | 7450 | CA | PRO | A 951 | 59.812 | 51.339 | −25.506 | 1.00 | 0.00 | C |
| ATOM | 7451 | C | PRO | A 951 | 58.485 | 52.094 | −25.625 | 1.00 | 0.00 | C |
| ATOM | 7452 | O | PRO | A 951 | 58.062 | 52.770 | −24.685 | 1.00 | 0.00 | O |
| ATOM | 7453 | CB | PRO | A 951 | 59.682 | 50.080 | −24.648 | 1.00 | 0.00 | C |
| ATOM | 7454 | CG | PRO | A 951 | 61.045 | 49.955 | −24.029 | 1.00 | 0.00 | C |
| ATOM | 7455 | CD | PRO | A 951 | 61.345 | 51.375 | −23.634 | 1.00 | 0.00 | C |
| ATOM | 7456 | N | SER | A 952 | 57.841 | 51.994 | −26.783 | 1.00 | 0.00 | N |
| ATOM | 7457 | CA | SER | A 952 | 56.560 | 52.660 | −27.001 | 1.00 | 0.00 | C |
| ATOM | 7458 | C | SER | A 952 | 55.488 | 51.599 | −26.794 | 1.00 | 0.00 | C |
| ATOM | 7459 | O | SER | A 952 | 55.184 | 50.823 | −27.704 | 1.00 | 0.00 | O |
| ATOM | 7460 | CB | SER | A 952 | 56.487 | 53.225 | −28.423 | 1.00 | 0.00 | C |
| ATOM | 7461 | OG | SER | A 952 | 55.404 | 54.129 | −28.568 | 1.00 | 0.00 | O |
| ATOM | 7462 | N | ALA | A 953 | 54.921 | 51.575 | −25.589 | 1.00 | 0.00 | N |
| ATOM | 7463 | CA | ALA | A 953 | 53.913 | 50.590 | −25.212 | 1.00 | 0.00 | C |
| ATOM | 7464 | C | ALA | A 953 | 52.550 | 50.732 | −25.872 | 1.00 | 0.00 | C |
| ATOM | 7465 | O | ALA | A 953 | 52.175 | 51.807 | −26.342 | 1.00 | 0.00 | O |
| ATOM | 7466 | CB | ALA | A 953 | 53.741 | 50.592 | −23.697 | 1.00 | 0.00 | C |
| ATOM | 7467 | N | ARG | A 954 | 51.811 | 49.625 | −25.876 | 1.00 | 0.00 | N |
| ATOM | 7468 | CA | ARG | A 954 | 50.473 | 49.573 | −26.447 | 1.00 | 0.00 | C |
| ATOM | 7469 | C | ARG | A 954 | 49.639 | 50.690 | −25.825 | 1.00 | 0.00 | C |
| ATOM | 7470 | O | ARG | A 954 | 49.742 | 50.963 | −24.625 | 1.00 | 0.00 | O |
| ATOM | 7471 | CB | ARG | A 954 | 49.854 | 48.203 | −26.173 | 1.00 | 0.00 | C |
| ATOM | 7472 | CG | ARG | A 954 | 48.507 | 47.969 | −26.831 | 1.00 | 0.00 | C |
| ATOM | 7473 | CD | ARG | A 954 | 48.382 | 46.513 | −27.253 | 1.00 | 0.00 | C |
| ATOM | 7474 | NE | ARG | A 954 | 46.995 | 46.082 | −27.379 | 1.00 | 0.00 | N |
| ATOM | 7475 | CZ | ARG | A 954 | 46.197 | 45.837 | −26.346 | 1.00 | 0.00 | C |
| ATOM | 7476 | NH1 | ARG | A 954 | 46.655 | 45.981 | −25.111 | 1.00 | 0.00 | N |
| ATOM | 7477 | NH2 | ARG | A 954 | 44.946 | 45.442 | −26.545 | 1.00 | 0.00 | N |
| ATOM | 7478 | N | GLU | A 955 | 48.805 | 51.320 | −26.648 | 1.00 | 0.00 | N |
| ATOM | 7479 | CA | GLU | A 955 | 47.993 | 52.460 | −26.225 | 1.00 | 0.00 | C |
| ATOM | 7480 | C | GLU | A 955 | 47.134 | 52.324 | −24.968 | 1.00 | 0.00 | C |
| ATOM | 7481 | O | GLU | A 955 | 46.846 | 53.324 | −24.319 | 1.00 | 0.00 | O |
| ATOM | 7482 | CB | GLU | A 955 | 47.108 | 52.922 | −27.385 | 1.00 | 0.00 | C |
| ATOM | 7483 | CG | GLU | A 955 | 45.917 | 52.029 | −27.646 | 1.00 | 0.00 | C |
| ATOM | 7484 | CD | GLU | A 955 | 45.090 | 52.504 | −28.820 | 1.00 | 0.00 | C |
| ATOM | 7485 | OE1 | GLU | A 955 | 44.949 | 53.734 | −28.996 | 1.00 | 0.00 | O |
| ATOM | 7486 | OE2 | GLU | A 955 | 44.574 | 51.648 | −29.560 | 1.00 | 0.00 | O |
| ATOM | 7487 | N | ASP | A 956 | 46.719 | 51.112 | −24.618 | 1.00 | 0.00 | N |
| ATOM | 7488 | CA | ASP | A 956 | 45.889 | 50.954 | −23.426 | 1.00 | 0.00 | C |
| ATOM | 7489 | C | ASP | A 956 | 46.712 | 50.774 | −22.152 | 1.00 | 0.00 | C |
| ATOM | 7490 | O | ASP | A 956 | 46.160 | 50.585 | −21.068 | 1.00 | 0.00 | O |
| ATOM | 7491 | CB | ASP | A 956 | 44.895 | 49.789 | −23.596 | 1.00 | 0.00 | C |
| ATOM | 7492 | CG | ASP | A 956 | 45.574 | 48.459 | −23.883 | 1.00 | 0.00 | C |
| ATOM | 7493 | OD1 | ASP | A 956 | 46.818 | 48.417 | −23.988 | 1.00 | 0.00 | O |
| ATOM | 7494 | OD2 | ASP | A 956 | 44.848 | 47.445 | −24.006 | 1.00 | 0.00 | O |
| ATOM | 7495 | N | LEU | A 957 | 48.033 | 50.849 | −22.280 | 1.00 | 0.00 | N |
| ATOM | 7496 | CA | LEU | A 957 | 48.907 | 50.701 | −21.121 | 1.00 | 0.00 | C |
| ATOM | 7497 | C | LEU | A 957 | 49.501 | 52.049 | −20.728 | 1.00 | 0.00 | C |
| ATOM | 7498 | O | LEU | A 957 | 49.847 | 52.858 | −21.588 | 1.00 | 0.00 | O |
| ATOM | 7499 | CB | LEU | A 957 | 50.042 | 49.718 | −21.426 | 1.00 | 0.00 | C |
| ATOM | 7500 | CG | LEU | A 957 | 50.902 | 49.265 | −20.240 | 1.00 | 0.00 | C |
| ATOM | 7501 | CD1 | LEU | A 957 | 50.042 | 48.498 | −19.238 | 1.00 | 0.00 | C |
| ATOM | 7502 | CD2 | LEU | A 957 | 52.037 | 48.377 | −20.736 | 1.00 | 0.00 | C |
| ATOM | 7503 | N | ASP | A 958 | 49.614 | 52.289 | −19.427 | 1.00 | 0.00 | N |
| ATOM | 7504 | CA | ASP | A 958 | 50.192 | 53.535 | −18.942 | 1.00 | 0.00 | C |
| ATOM | 7505 | C | ASP | A 958 | 51.058 | 53.276 | −17.719 | 1.00 | 0.00 | C |
| ATOM | 7506 | O | ASP | A 958 | 50.813 | 52.333 | −16.965 | 1.00 | 0.00 | O |
| ATOM | 7507 | CB | ASP | A 958 | 49.083 | 54.537 | −18.583 | 1.00 | 0.00 | C |
| ATOM | 7508 | CG | ASP | A 958 | 49.628 | 55.918 | −18.229 | 1.00 | 0.00 | C |
| ATOM | 7509 | OD1 | ASP | A 958 | 50.713 | 56.272 | −18.732 | 1.00 | 0.00 | O |
| ATOM | 7510 | OD2 | ASP | A 958 | 48.962 | 56.652 | −17.463 | 1.00 | 0.00 | O |
| ATOM | 7511 | N | VAL | A 959 | 52.101 | 54.083 | −17.561 | 1.00 | 0.00 | N |
| ATOM | 7512 | CA | VAL | A 959 | 52.967 | 54.006 | −16.390 | 1.00 | 0.00 | C |
| ATOM | 7513 | C | VAL | A 959 | 52.402 | 55.141 | −15.539 | 1.00 | 0.00 | C |
| ATOM | 7514 | O | VAL | A 959 | 52.820 | 56.294 | −15.658 | 1.00 | 0.00 | O |
| ATOM | 7515 | CB | VAL | A 959 | 54.445 | 54.299 | −16.738 | 1.00 | 0.00 | C |
| ATOM | 7516 | CG1 | VAL | A 959 | 55.279 | 54.442 | −15.447 | 1.00 | 0.00 | C |
| ATOM | 7517 | CG2 | VAL | A 959 | 55.005 | 53.168 | −17.593 | 1.00 | 0.00 | C |
| ATOM | 7518 | N | SER | A 960 | 51.416 | 54.804 | −14.713 | 1.00 | 0.00 | N |
| ATOM | 7519 | CA | SER | A 960 | 50.740 | 55.773 | −13.852 | 1.00 | 0.00 | C |
| ATOM | 7520 | C | SER | A 960 | 51.688 | 56.483 | −12.898 | 1.00 | 0.00 | C |
| ATOM | 7521 | O | SER | A 960 | 51.542 | 57.682 | −12.624 | 1.00 | 0.00 | O |
| ATOM | 7522 | CB | SER | A 960 | 49.646 | 55.066 | −13.046 | 1.00 | 0.00 | C |
| ATOM | 7523 | OG | SER | A 960 | 48.818 | 54.290 | −13.898 | 1.00 | 0.00 | O |
| ATOM | 7524 | N | VAL | A 961 | 52.650 | 55.729 | −12.383 | 1.00 | 0.00 | N |
| ATOM | 7525 | CA | VAL | A 961 | 53.624 | 56.267 | −11.446 | 1.00 | 0.00 | C |
| ATOM | 7526 | C | VAL | A 961 | 55.009 | 55.683 | −11.677 | 1.00 | 0.00 | C |
| ATOM | 7527 | O | VAL | A 961 | 55.161 | 54.481 | −11.924 | 1.00 | 0.00 | O |
| ATOM | 7528 | CB | VAL | A 961 | 53.240 | 55.948 | −9.969 | 1.00 | 0.00 | C |
| ATOM | 7529 | CG1 | VAL | A 961 | 54.339 | 56.448 | −9.018 | 1.00 | 0.00 | C |
| ATOM | 7530 | CG2 | VAL | A 961 | 51.899 | 56.589 | −9.613 | 1.00 | 0.00 | C |
| ATOM | 7531 | N | MET | A 962 | 56.006 | 56.560 | −11.622 | 1.00 | 0.00 | N |
| ATOM | 7532 | CA | MET | A 962 | 57.401 | 56.171 | −11.712 | 1.00 | 0.00 | C |
| ATOM | 7533 | C | MET | A 962 | 58.003 | 56.955 | −10.560 | 1.00 | 0.00 | C |
| ATOM | 7534 | O | MET | A 962 | 57.976 | 58.188 | −10.559 | 1.00 | 0.00 | O |
| ATOM | 7535 | CB | MET | A 962 | 58.056 | 56.601 | −13.021 | 1.00 | 0.00 | C |
| ATOM | 7536 | CG | MET | A 962 | 59.529 | 56.213 | −13.047 | 1.00 | 0.00 | C |
| ATOM | 7537 | SD | MET | A 962 | 60.355 | 56.541 | −14.598 | 1.00 | 0.00 | S |
| ATOM | 7538 | CE | MET | A 962 | 62.029 | 55.938 | −14.241 | 1.00 | 0.00 | C |
| ATOM | 7539 | N | ARG | A 963 | 58.528 | 56.246 | −9.568 | 1.00 | 0.00 | N |
| ATOM | 7540 | CA | ARG | A 963 | 59.081 | 56.906 | −8.393 | 1.00 | 0.00 | C |
| ATOM | 7541 | C | ARG | A 963 | 60.306 | 56.201 | −7.847 | 1.00 | 0.00 | C |
| ATOM | 7542 | O | ARG | A 963 | 60.242 | 55.020 | −7.519 | 1.00 | 0.00 | O |
| ATOM | 7543 | CB | ARG | A 963 | 58.005 | 56.965 | −7.294 | 1.00 | 0.00 | C |
| ATOM | 7544 | CG | ARG | A 963 | 58.486 | 57.450 | −5.919 | 1.00 | 0.00 | C |
| ATOM | 7545 | CD | ARG | A 963 | 57.362 | 57.330 | −4.876 | 1.00 | 0.00 | C |
| ATOM | 7546 | NE | ARG | A 963 | 56.174 | 58.055 | −5.321 | 1.00 | 0.00 | N |
| ATOM | 7547 | CZ | ARG | A 963 | 54.929 | 57.597 | −5.238 | 1.00 | 0.00 | C |
| ATOM | 7548 | NH1 | ARG | A 963 | 54.679 | 56.405 | −4.707 | 1.00 | 0.00 | N |
| ATOM | 7549 | NH2 | ARG | A 963 | 53.935 | 58.317 | −5.738 | 1.00 | 0.00 | N |
| ATOM | 7550 | N | ARG | A 964 | 61.423 | 56.918 | −7.755 | 1.00 | 0.00 | N |
| ATOM | 7551 | CA | ARG | A 964 | 62.628 | 56.322 | −7.186 | 1.00 | 0.00 | C |
| ATOM | 7552 | C | ARG | A 964 | 62.316 | 56.255 | −5.695 | 1.00 | 0.00 | C |
| ATOM | 7553 | O | ARG | A 964 | 61.881 | 57.245 | −5.100 | 1.00 | 0.00 | O |
| ATOM | 7554 | CB | ARG | A 964 | 63.860 | 57.197 | −7.442 | 1.00 | 0.00 | C |
| ATOM | 7555 | CG | ARG | A 964 | 65.152 | 56.585 | −6.894 | 1.00 | 0.00 | C |
| ATOM | 7556 | CD | ARG | A 964 | 66.386 | 57.327 | −7.393 | 1.00 | 0.00 | C |
| ATOM | 7557 | NE | ARG | A 964 | 66.594 | 57.152 | −8.830 | 1.00 | 0.00 | N |
| ATOM | 7558 | CZ | ARG | A 964 | 67.472 | 56.313 | −9.371 | 1.00 | 0.00 | C |
| ATOM | 7559 | NH1 | ARG | A 964 | 68.242 | 55.554 | −8.600 | 1.00 | 0.00 | N |
| ATOM | 7560 | NH2 | ARG | A 964 | 67.596 | 56.243 | −10.690 | 1.00 | 0.00 | N |
| ATOM | 7561 | N | LEU | A 965 | 62.528 | 55.088 | −5.098 | 1.00 | 0.00 | N |
| ATOM | 7562 | CA | LEU | A 965 | 62.204 | 54.869 | −3.691 | 1.00 | 0.00 | C |
| ATOM | 7563 | C | LEU | A 965 | 63.382 | 54.975 | −2.728 | 1.00 | 0.00 | C |
| ATOM | 7564 | O | LEU | A 965 | 63.199 | 54.897 | −1.514 | 1.00 | 0.00 | O |
| ATOM | 7565 | CB | LEU | A 965 | 61.552 | 53.491 | −3.540 | 1.00 | 0.00 | C |
| ATOM | 7566 | CG | LEU | A 965 | 60.339 | 53.218 | −4.440 | 1.00 | 0.00 | C |
| ATOM | 7567 | CD1 | LEU | A 965 | 59.998 | 51.737 | −4.413 | 1.00 | 0.00 | C |
| ATOM | 7568 | CD2 | LEU | A 965 | 59.151 | 54.063 | −3.981 | 1.00 | 0.00 | C |
| ATOM | 7569 | N | THR | A 966 | 64.582 | 55.160 | −3.270 | 1.00 | 0.00 | N |
| ATOM | 7570 | CA | THR | A 966 | 65.780 | 55.255 | −2.445 | 1.00 | 0.00 | C |
| ATOM | 7571 | C | THR | A 966 | 66.526 | 56.568 | −2.622 | 1.00 | 0.00 | C |
| ATOM | 7572 | O | THR | A 966 | 66.500 | 57.170 | −3.698 | 1.00 | 0.00 | O |
| ATOM | 7573 | CB | THR | A 966 | 66.767 | 54.115 | −2.776 | 1.00 | 0.00 | C |
| ATOM | 7574 | OG1 | THR | A 966 | 66.988 | 54.074 | −4.191 | 1.00 | 0.00 | O |
| ATOM | 7575 | CG2 | THR | A 966 | 66.218 | 52.773 | −2.311 | 1.00 | 0.00 | C |
| ATOM | 7576 | N | LYS | A 967 | 67.190 | 57.006 | −1.558 | 1.00 | 0.00 | N |
| ATOM | 7577 | CA | LYS | A 967 | 67.987 | 58.223 | −1.608 | 1.00 | 0.00 | C |
| ATOM | 7578 | C | LYS | A 967 | 69.364 | 57.836 | −2.157 | 1.00 | 0.00 | C |
| ATOM | 7579 | O | LYS | A 967 | 69.675 | 56.650 | −2.275 | 1.00 | 0.00 | O |
| ATOM | 7580 | CB | LYS | A 967 | 68.098 | 58.849 | −0.217 | 1.00 | 0.00 | C |
| ATOM | 7581 | CG | LYS | A 967 | 66.760 | 59.367 | 0.313 | 1.00 | 0.00 | C |
| ATOM | 7582 | CD | LYS | A 967 | 66.947 | 60.187 | 1.577 | 1.00 | 0.00 | C |
| ATOM | 7583 | CE | LYS | A 967 | 65.638 | 60.793 | 2.048 | 1.00 | 0.00 | C |
| ATOM | 7584 | NZ | LYS | A 967 | 65.850 | 61.621 | 3.269 | 1.00 | 0.00 | N |
| ATOM | 7585 | N | SER | A 968 | 70.185 | 58.829 | −2.488 | 1.00 | 0.00 | N |
| ATOM | 7586 | CA | SER | A 968 | 71.499 | 58.577 | −3.078 | 1.00 | 0.00 | C |
| ATOM | 7587 | C | SER | A 968 | 72.484 | 57.730 | −2.270 | 1.00 | 0.00 | C |
| ATOM | 7588 | O | SER | A 968 | 73.349 | 57.079 | −2.850 | 1.00 | 0.00 | O |
| ATOM | 7589 | CB | SER | A 968 | 72.175 | 59.904 | −3.447 | 1.00 | 0.00 | C |
| ATOM | 7590 | OG | SER | A 968 | 72.506 | 60.651 | −2.291 | 1.00 | 0.00 | O |
| ATOM | 7591 | N | SER | A 969 | 72.355 | 57.725 | −0.948 | 1.00 | 0.00 | N |
| ATOM | 7592 | CA | SER | A 969 | 73.279 | 56.957 | −0.109 | 1.00 | 0.00 | C |
| ATOM | 7593 | C | SER | A 969 | 73.058 | 55.444 | −0.131 | 1.00 | 0.00 | C |
| ATOM | 7594 | O | SER | A 969 | 73.910 | 54.684 | 0.334 | 1.00 | 0.00 | O |
| ATOM | 7595 | CB | SER | A 969 | 73.215 | 57.454 | 1.337 | 1.00 | 0.00 | C |
| ATOM | 7596 | OG | SER | A 969 | 71.918 | 57.274 | 1.878 | 1.00 | 0.00 | O |
| ATOM | 7597 | N | ALA | A 970 | 71.926 | 55.005 | −0.675 | 1.00 | 0.00 | N |
| ATOM | 7598 | CA | ALA | A 970 | 71.612 | 53.578 | −0.738 | 1.00 | 0.00 | C |
| ATOM | 7599 | C | ALA | A 970 | 72.428 | 52.823 | −1.785 | 1.00 | 0.00 | C |
| ATOM | 7600 | O | ALA | A 970 | 72.368 | 53.135 | −2.978 | 1.00 | 0.00 | O |
| ATOM | 7601 | CB | ALA | A 970 | 70.121 | 53.385 | −1.009 | 1.00 | 0.00 | C |
| ATOM | 7602 | N | LYS | A 971 | 73.186 | 51.825 | −1.335 | 1.00 | 0.00 | N |
| ATOM | 7603 | CA | LYS | A 971 | 74.002 | 51.014 | −2.236 | 1.00 | 0.00 | C |
| ATOM | 7604 | C | LYS | A 971 | 73.142 | 50.415 | −3.337 | 1.00 | 0.00 | C |
| ATOM | 7605 | O | LYS | A 971 | 73.517 | 50.429 | −4.507 | 1.00 | 0.00 | O |
| ATOM | 7606 | CB | LYS | A 971 | 74.688 | 49.880 | −1.470 | 1.00 | 0.00 | C |
| ATOM | 7607 | CG | LYS | A 971 | 76.025 | 50.250 | −0.843 | 1.00 | 0.00 | C |
| ATOM | 7608 | CD | LYS | A 971 | 76.622 | 49.070 | −0.082 | 1.00 | 0.00 | C |
| ATOM | 7609 | CE | LYS | A 971 | 76.665 | 47.808 | −0.941 | 1.00 | 0.00 | C |
| ATOM | 7610 | NZ | LYS | A 971 | 77.418 | 47.998 | −2.210 | 1.00 | 0.00 | N |
| ATOM | 7611 | N | THR | A 972 | 71.993 | 49.868 | −2.953 | 1.00 | 0.00 | N |
| ATOM | 7612 | CA | THR | A 972 | 71.083 | 49.283 | −3.922 | 1.00 | 0.00 | C |
| ATOM | 7613 | C | THR | A 972 | 69.931 | 50.255 | −4.156 | 1.00 | 0.00 | C |
| ATOM | 7614 | O | THR | A 972 | 69.110 | 50.493 | −3.267 | 1.00 | 0.00 | O |
| ATOM | 7615 | CB | THR | A 972 | 70.523 | 47.921 | −3.432 | 1.00 | 0.00 | C |
| ATOM | 7616 | OG1 | THR | A 972 | 71.603 | 46.991 | −3.271 | 1.00 | 0.00 | O |
| ATOM | 7617 | CG2 | THR | A 972 | 69.538 | 47.349 | −4.446 | 1.00 | 0.00 | C |
| ATOM | 7618 | N | GLN | A 973 | 69.890 | 50.833 | −5.351 | 1.00 | 0.00 | N |
| ATOM | 7619 | CA | GLN | A 973 | 68.836 | 51.774 | −5.697 | 1.00 | 0.00 | C |
| ATOM | 7620 | C | GLN | A 973 | 67.568 | 51.029 | −6.081 | 1.00 | 0.00 | C |
| ATOM | 7621 | O | GLN | A 973 | 67.621 | 49.944 | −6.662 | 1.00 | 0.00 | O |
| ATOM | 7622 | CB | GLN | A 973 | 69.278 | 52.674 | −6.854 | 1.00 | 0.00 | C |
| ATOM | 7623 | CG | GLN | A 973 | 70.379 | 53.652 | −6.481 | 1.00 | 0.00 | C |
| ATOM | 7624 | CD | GLN | A 973 | 69.945 | 54.650 | −5.426 | 1.00 | 0.00 | C |
| ATOM | 7625 | OE1 | GLN | A 973 | 70.607 | 54.812 | −4.397 | 1.00 | 0.00 | O |
| ATOM | 7626 | NE2 | GLN | A 973 | 68.836 | 55.334 | −5.678 | 1.00 | 0.00 | N |
| ATOM | 7627 | N | ARG | A 974 | 66.427 | 51.620 | −5.749 | 1.00 | 0.00 | N |
| ATOM | 7628 | CA | ARG | A 974 | 65.145 | 51.015 | −6.063 | 1.00 | 0.00 | C |
| ATOM | 7629 | C | ARG | A 974 | 64.220 | 52.030 | −6.722 | 1.00 | 0.00 | C |
| ATOM | 7630 | O | ARG | A 974 | 64.128 | 53.187 | −6.289 | 1.00 | 0.00 | O |
| ATOM | 7631 | CB | ARG | A 974 | 64.497 | 50.472 | −4.788 | 1.00 | 0.00 | C |
| ATOM | 7632 | CG | ARG | A 974 | 65.322 | 49.406 | −4.067 | 1.00 | 0.00 | C |
| ATOM | 7633 | CD | ARG | A 974 | 64.717 | 49.072 | −2.701 | 1.00 | 0.00 | C |
| ATOM | 7634 | NE | ARG | A 974 | 63.452 | 48.345 | −2.805 | 1.00 | 0.00 | N |
| ATOM | 7635 | CZ | ARG | A 974 | 62.321 | 48.718 | −2.211 | 1.00 | 0.00 | C |
| ATOM | 7636 | NH1 | ARG | A 974 | 62.286 | 49.817 | −1.470 | 1.00 | 0.00 | N |
| ATOM | 7637 | NH2 | ARG | A 974 | 61.223 | 47.986 | −2.351 | 1.00 | 0.00 | N |
| ATOM | 7638 | N | VAL | A 975 | 63.544 | 51.593 | −7.778 | 1.00 | 0.00 | N |
| ATOM | 7639 | CA | VAL | A 975 | 62.613 | 52.450 | −8.496 | 1.00 | 0.00 | C |
| ATOM | 7640 | C | VAL | A 975 | 61.291 | 51.709 | −8.620 | 1.00 | 0.00 | C |
| ATOM | 7641 | O | VAL | A 975 | 61.246 | 50.564 | −9.079 | 1.00 | 0.00 | O |
| ATOM | 7642 | CB | VAL | A 975 | 63.148 | 52.810 | −9.903 | 1.00 | 0.00 | C |
| ATOM | 7643 | CG1 | VAL | A 975 | 62.159 | 53.728 | −10.624 | 1.00 | 0.00 | C |
| ATOM | 7644 | CG2 | VAL | A 975 | 64.498 | 53.498 | −9.775 | 1.00 | 0.00 | C |
| ATOM | 7645 | N | GLY | A 976 | 60.217 | 52.365 | −8.192 | 1.00 | 0.00 | N |
| ATOM | 7646 | CA | GLY | A 976 | 58.906 | 51.748 | −8.249 | 1.00 | 0.00 | C |
| ATOM | 7647 | C | GLY | A 976 | 58.062 | 52.237 | −9.407 | 1.00 | 0.00 | C |
| ATOM | 7648 | O | GLY | A 976 | 58.132 | 53.406 | −9.804 | 1.00 | 0.00 | O |
| ATOM | 7649 | N | TYR | A 977 | 57.261 | 51.328 | −9.947 | 1.00 | 0.00 | N |
| ATOM | 7650 | CA | TYR | A 977 | 56.380 | 51.637 | −11.062 | 1.00 | 0.00 | C |
| ATOM | 7651 | C | TYR | A 977 | 54.980 | 51.110 | −10.823 | 1.00 | 0.00 | C |
| ATOM | 7652 | O | TYR | A 977 | 54.803 | 49.991 | −10.344 | 1.00 | 0.00 | O |
| ATOM | 7653 | CB | TYR | A 977 | 56.871 | 50.989 | −12.360 | 1.00 | 0.00 | C |
| ATOM | 7654 | CG | TYR | A 977 | 58.264 | 51.359 | −12.787 | 1.00 | 0.00 | C |
| ATOM | 7655 | CD1 | TYR | A 977 | 59.373 | 50.677 | −12.287 | 1.00 | 0.00 | C |
| ATOM | 7656 | CD2 | TYR | A 977 | 58.478 | 52.388 | −13.702 | 1.00 | 0.00 | C |
| ATOM | 7657 | CE1 | TYR | A 977 | 60.668 | 51.014 | −12.695 | 1.00 | 0.00 | C |
| ATOM | 7658 | CE2 | TYR | A 977 | 59.759 | 52.734 | −14.112 | 1.00 | 0.00 | C |
| ATOM | 7659 | CZ | TYR | A 977 | 60.849 | 52.048 | −13.609 | 1.00 | 0.00 | C |
| ATOM | 7660 | OH | TYR | A 977 | 62.118 | 52.407 | −14.016 | 1.00 | 0.00 | O |
| ATOM | 7661 | N | VAL | A 978 | 53.984 | 51.921 | −11.160 | 1.00 | 0.00 | N |
| ATOM | 7662 | CA | VAL | A 978 | 52.606 | 51.484 | −11.061 | 1.00 | 0.00 | C |
| ATOM | 7663 | C | VAL | A 978 | 52.145 | 51.449 | −12.514 | 1.00 | 0.00 | C |
| ATOM | 7664 | O | VAL | A 978 | 52.166 | 52.468 | −13.209 | 1.00 | 0.00 | O |
| ATOM | 7665 | CB | VAL | A 978 | 51.728 | 52.454 | −10.253 | 1.00 | 0.00 | C |
| ATOM | 7666 | CG1 | VAL | A 978 | 50.265 | 52.036 | −10.369 | 1.00 | 0.00 | C |
| ATOM | 7667 | CG2 | VAL | A 978 | 52.156 | 52.438 | −8.786 | 1.00 | 0.00 | C |
| ATOM | 7668 | N | LEU | A 979 | 51.775 | 50.258 | −12.972 | 1.00 | 0.00 | N |
| ATOM | 7669 | CA | LEU | A 979 | 51.327 | 50.050 | −14.341 | 1.00 | 0.00 | C |
| ATOM | 7670 | C | LEU | A 979 | 49.838 | 49.788 | −14.410 | 1.00 | 0.00 | C |
| ATOM | 7671 | O | LEU | A 979 | 49.331 | 48.875 | −13.764 | 1.00 | 0.00 | O |
| ATOM | 7672 | CB | LEU | A 979 | 52.061 | 48.859 | −14.963 | 1.00 | 0.00 | C |
| ATOM | 7673 | CG | LEU | A 979 | 53.383 | 49.114 | −15.689 | 1.00 | 0.00 | C |
| ATOM | 7674 | CD1 | LEU | A 979 | 53.099 | 49.753 | −17.036 | 1.00 | 0.00 | C |
| ATOM | 7675 | CD2 | LEU | A 979 | 54.285 | 50.000 | −14.840 | 1.00 | 0.00 | C |
| ATOM | 7676 | N | HIS | A 980 | 49.137 | 50.589 | −15.201 | 1.00 | 0.00 | N |
| ATOM | 7677 | CA | HIS | A 980 | 47.710 | 50.391 | −15.351 | 1.00 | 0.00 | C |
| ATOM | 7678 | C | HIS | A 980 | 47.335 | 50.154 | −16.801 | 1.00 | 0.00 | C |
| ATOM | 7679 | O | HIS | A 980 | 47.783 | 50.870 | −17.693 | 1.00 | 0.00 | O |
| ATOM | 7680 | CB | HIS | A 980 | 46.920 | 51.591 | −14.830 | 1.00 | 0.00 | C |
| ATOM | 7681 | CG | HIS | A 980 | 45.441 | 51.436 | −15.001 | 1.00 | 0.00 | C |
| ATOM | 7682 | ND1 | HIS | A 980 | 44.744 | 52.017 | −16.039 | 1.00 | 0.00 | N |
| ATOM | 7683 | CD2 | HIS | A 980 | 44.543 | 50.691 | −14.316 | 1.00 | 0.00 | C |
| ATOM | 7684 | CE2 | HIS | A 980 | 43.480 | 51.634 | −15.987 | 1.00 | 0.00 | C |
| ATOM | 7685 | NE2 | HIS | A 980 | 43.332 | 50.828 | −14.951 | 1.00 | 0.00 | N |
| ATOM | 7686 | N | ARG | A 981 | 46.519 | 49.133 | −17.029 | 1.00 | 0.00 | N |
| ATOM | 7687 | CA | ARG | A 981 | 46.051 | 48.831 | −18.370 | 1.00 | 0.00 | C |
| ATOM | 7688 | C | ARG | A 981 | 44.539 | 48.974 | −18.352 | 1.00 | 0.00 | C |
| ATOM | 7689 | O | ARG | A 981 | 43.848 | 48.296 | −17.593 | 1.00 | 0.00 | O |
| ATOM | 7690 | CB | ARG | A 981 | 46.438 | 47.413 | −18.779 | 1.00 | 0.00 | C |
| ATOM | 7691 | CG | ARG | A 981 | 46.065 | 47.071 | −20.214 | 1.00 | 0.00 | C |
| ATOM | 7692 | CD | ARG | A 981 | 46.612 | 45.714 | −20.606 | 1.00 | 0.00 | C |
| ATOM | 7693 | NE | ARG | A 981 | 46.188 | 45.321 | −21.944 | 1.00 | 0.00 | N |
| ATOM | 7694 | CZ | ARG | A 981 | 46.440 | 44.131 | −22.485 | 1.00 | 0.00 | C |
| ATOM | 7695 | NH1 | ARG | A 981 | 47.116 | 43.217 | −21.798 | 1.00 | 0.00 | N |
| ATOM | 7696 | NH2 | ARG | A 981 | 46.011 | 43.850 | −23.709 | 1.00 | 0.00 | N |
| ATOM | 7697 | N | THR | A 982 | 44.029 | 49.883 | −19.170 | 1.00 | 0.00 | N |
| ATOM | 7698 | CA | THR | A 982 | 42.595 | 50.108 | −19.253 | 1.00 | 0.00 | C |
| ATOM | 7699 | C | THR | A 982 | 42.070 | 49.120 | −20.293 | 1.00 | 0.00 | C |
| ATOM | 7700 | O | THR | A 982 | 42.813 | 48.248 | −20.744 | 1.00 | 0.00 | O |
| ATOM | 7701 | CB | THR | A 982 | 42.301 | 51.560 | −19.698 | 1.00 | 0.00 | C |
| ATOM | 7702 | OG1 | THR | A 982 | 40.898 | 51.824 | −19.601 | 1.00 | 0.00 | O |
| ATOM | 7703 | CG2 | THR | A 982 | 42.769 | 51.787 | −21.134 | 1.00 | 0.00 | C |
| ATOM | 7704 | N | ASN | A 983 | 40.795 | 49.232 | −20.652 | 1.00 | 0.00 | N |
| ATOM | 7705 | CA | ASN | A 983 | 40.232 | 48.351 | −21.668 | 1.00 | 0.00 | C |
| ATOM | 7706 | C | ASN | A 983 | 39.540 | 49.217 | −22.704 | 1.00 | 0.00 | C |
| ATOM | 7707 | O | ASN | A 983 | 38.621 | 49.963 | −22.378 | 1.00 | 0.00 | O |
| ATOM | 7708 | CB | ASN | A 983 | 39.212 | 47.374 | −21.080 | 1.00 | 0.00 | C |
| ATOM | 7709 | CG | ASN | A 983 | 38.715 | 46.375 | −22.114 | 1.00 | 0.00 | C |
| ATOM | 7710 | OD1 | ASN | A 983 | 39.467 | 45.514 | −22.565 | 1.00 | 0.00 | O |
| ATOM | 7711 | ND2 | ASN | A 983 | 37.450 | 46.499 | −22.506 | 1.00 | 0.00 | N |
| ATOM | 7712 | N | LEU | A 984 | 39.993 | 49.116 | −23.947 | 1.00 | 0.00 | N |
| ATOM | 7713 | CA | LEU | A 984 | 39.424 | 49.894 | −25.038 | 1.00 | 0.00 | C |
| ATOM | 7714 | C | LEU | A 984 | 38.640 | 48.978 | −25.958 | 1.00 | 0.00 | C |
| ATOM | 7715 | O | LEU | A 984 | 39.061 | 47.857 | −26.231 | 1.00 | 0.00 | O |
| ATOM | 7716 | CB | LEU | A 984 | 40.535 | 50.579 | −25.829 | 1.00 | 0.00 | C |
| ATOM | 7717 | CG | LEU | A 984 | 41.504 | 51.430 | −25.003 | 1.00 | 0.00 | C |
| ATOM | 7718 | CD1 | LEU | A 984 | 42.577 | 51.997 | −25.922 | 1.00 | 0.00 | C |
| ATOM | 7719 | CD2 | LEU | A 984 | 40.742 | 52.545 | −24.289 | 1.00 | 0.00 | C |
| ATOM | 7720 | N | MET | A 985 | 37.499 | 49.456 | −26.435 | 1.00 | 0.00 | N |
| ATOM | 7721 | CA | MET | A 985 | 36.674 | 48.652 | −27.324 | 1.00 | 0.00 | C |
| ATOM | 7722 | C | MET | A 985 | 37.356 | 48.361 | −28.648 | 1.00 | 0.00 | C |
| ATOM | 7723 | O | MET | A 985 | 38.050 | 49.210 | −29.210 | 1.00 | 0.00 | O |
| ATOM | 7724 | CB | MET | A 985 | 35.347 | 49.349 | −27.612 | 1.00 | 0.00 | C |
| ATOM | 7725 | CG | MET | A 985 | 34.437 | 49.487 | −26.419 | 1.00 | 0.00 | C |
| ATOM | 7726 | SD | MET | A 985 | 32.757 | 49.769 | −26.967 | 1.00 | 0.00 | S |
| ATOM | 7727 | CE | MET | A 985 | 32.914 | 51.378 | −27.737 | 1.00 | 0.00 | C |
| ATOM | 7728 | N | GLN | A 986 | 37.155 | 47.148 | −29.142 | 1.00 | 0.00 | N |
| ATOM | 7729 | CA | GLN | A 986 | 37.711 | 46.760 | −30.424 | 1.00 | 0.00 | C |
| ATOM | 7730 | C | GLN | A 986 | 36.615 | 47.068 | −31.434 | 1.00 | 0.00 | C |
| ATOM | 7731 | O | GLN | A 986 | 35.524 | 46.496 | −31.379 | 1.00 | 0.00 | O |
| ATOM | 7732 | CB | GLN | A 986 | 38.077 | 45.273 | −30.416 | 1.00 | 0.00 | C |
| ATOM | 7733 | CG | GLN | A 986 | 37.120 | 44.382 | −29.645 | 1.00 | 0.00 | C |
| ATOM | 7734 | CD | GLN | A 986 | 37.776 | 43.088 | −29.190 | 1.00 | 0.00 | C |
| ATOM | 7735 | OE1 | GLN | A 986 | 37.140 | 42.244 | −28.554 | 1.00 | 0.00 | O |
| ATOM | 7736 | NE2 | GLN | A 986 | 39.058 | 42.930 | −29.509 | 1.00 | 0.00 | N |
| ATOM | 7737 | N | CYS | A 987 | 36.900 | 48.001 | −32.335 | 1.00 | 0.00 | N |
| ATOM | 7738 | CA | CYS | A 987 | 35.922 | 48.410 | −33.331 | 1.00 | 0.00 | C |
| ATOM | 7739 | C | CYS | A 987 | 36.382 | 48.177 | −34.767 | 1.00 | 0.00 | C |
| ATOM | 7740 | O | CYS | A 987 | 35.880 | 48.819 | −35.693 | 1.00 | 0.00 | O |
| ATOM | 7741 | CB | CYS | A 987 | 35.583 | 49.886 | −33.143 | 1.00 | 0.00 | C |
| ATOM | 7742 | SG | CYS | A 987 | 35.118 | 50.371 | −31.449 | 1.00 | 0.00 | S |
| ATOM | 7743 | N | GLY | A 988 | 37.340 | 47.274 | −34.951 | 1.00 | 0.00 | N |
| ATOM | 7744 | CA | GLY | A 988 | 37.811 | 46.972 | −36.291 | 1.00 | 0.00 | C |
| ATOM | 7745 | C | GLY | A 988 | 39.100 | 47.636 | −36.732 | 1.00 | 0.00 | C |
| ATOM | 7746 | O | GLY | A 988 | 39.528 | 47.451 | −37.872 | 1.00 | 0.00 | O |
| ATOM | 7747 | N | THR | A 989 | 39.719 | 48.412 | −35.849 | 1.00 | 0.00 | N |
| ATOM | 7748 | CA | THR | A 989 | 40.972 | 49.077 | −36.187 | 1.00 | 0.00 | C |
| ATOM | 7749 | C | THR | A 989 | 42.145 | 48.151 | −35.880 | 1.00 | 0.00 | C |
| ATOM | 7750 | O | THR | A 989 | 42.332 | 47.724 | −34.739 | 1.00 | 0.00 | O |
| ATOM | 7751 | CB | THR | A 989 | 41.148 | 50.387 | −35.395 | 1.00 | 0.00 | C |
| ATOM | 7752 | OG1 | THR | A 989 | 40.102 | 51.301 | −35.747 | 1.00 | 0.00 | O |
| ATOM | 7753 | CG2 | THR | A 989 | 42.493 | 51.024 | −35.713 | 1.00 | 0.00 | C |
| ATOM | 7754 | N | PRO | A 990 | 42.950 | 47.824 | −36.905 | 1.00 | 0.00 | N |
| ATOM | 7755 | CA | PRO | A 990 | 44.115 | 46.944 | −36.777 | 1.00 | 0.00 | C |
| ATOM | 7756 | C | PRO | A 990 | 44.996 | 47.214 | −35.555 | 1.00 | 0.00 | C |
| ATOM | 7757 | O | PRO | A 990 | 45.331 | 46.286 | −34.816 | 1.00 | 0.00 | O |
| ATOM | 7758 | CB | PRO | A 990 | 44.851 | 47.169 | −38.092 | 1.00 | 0.00 | C |
| ATOM | 7759 | CG | PRO | A 990 | 43.719 | 47.347 | −39.054 | 1.00 | 0.00 | C |
| ATOM | 7760 | CD | PRO | A 990 | 42.799 | 48.287 | −38.297 | 1.00 | 0.00 | C |
| ATOM | 7761 | N | GLU | A 991 | 45.370 | 48.475 | −35.346 | 1.00 | 0.00 | N |
| ATOM | 7762 | CA | GLU | A 991 | 46.212 | 48.847 | −34.206 | 1.00 | 0.00 | C |
| ATOM | 7763 | C | GLU | A 991 | 47.358 | 47.848 | −34.040 | 1.00 | 0.00 | C |
| ATOM | 7764 | O | GLU | A 991 | 47.252 | 46.887 | −33.276 | 1.00 | 0.00 | O |
| ATOM | 7765 | CB | GLU | A 991 | 45.366 | 48.889 | −32.930 | 1.00 | 0.00 | C |
| ATOM | 7766 | CG | GLU | A 991 | 44.289 | 49.969 | −32.936 | 1.00 | 0.00 | C |
| ATOM | 7767 | CD | GLU | A 991 | 43.310 | 49.828 | −31.781 | 1.00 | 0.00 | C |
| ATOM | 7768 | OE1 | GLU | A 991 | 43.767 | 49.696 | −30.626 | 1.00 | 0.00 | O |
| ATOM | 7769 | OE2 | GLU | A 991 | 42.084 | 49.852 | −32.029 | 1.00 | 0.00 | O |
| ATOM | 7770 | N | GLU | A 992 | 48.458 | 48.086 | −34.747 | 1.00 | 0.00 | N |
| ATOM | 7771 | CA | GLU | A 992 | 49.600 | 47.181 | −34.695 | 1.00 | 0.00 | C |
| ATOM | 7772 | C | GLU | A 992 | 50.908 | 47.810 | −34.220 | 1.00 | 0.00 | C |
| ATOM | 7773 | O | GLU | A 992 | 50.939 | 48.929 | −33.704 | 1.00 | 0.00 | O |
| ATOM | 7774 | CB | GLU | A 992 | 49.825 | 46.565 | −36.078 | 1.00 | 0.00 | C |
| ATOM | 7775 | CG | GLU | A 992 | 49.602 | 45.879 | −36.669 | 1.00 | 0.00 | C |
| ATOM | 7776 | CD | GLU | A 992 | 48.828 | 45.426 | −38.101 | 1.00 | 0.00 | C |
| ATOM | 7777 | OE1 | GLU | A 992 | 49.733 | 44.593 | −38.330 | 1.00 | 0.00 | O |
| ATOM | 7778 | OE2 | GLU | A 992 | 48.102 | 45.906 | −38.999 | 1.00 | 0.00 | O |
| ATOM | 7779 | N | HIS | A 993 | 51.986 | 47.055 | −34.422 | 1.00 | 0.00 | N |
| ATOM | 7780 | CA | HIS | A 993 | 53.350 | 47.436 | −34.064 | 1.00 | 0.00 | C |
| ATOM | 7781 | C | HIS | A 993 | 53.557 | 48.283 | −32.810 | 1.00 | 0.00 | C |
| ATOM | 7782 | O | HIS | A 993 | 53.522 | 49.515 | −32.857 | 1.00 | 0.00 | O |
| ATOM | 7783 | CB | HIS | A 993 | 54.043 | 48.114 | −35.257 | 1.00 | 0.00 | C |
| ATOM | 7784 | CG | HIS | A 993 | 53.288 | 49.275 | −35.828 | 1.00 | 0.00 | C |
| ATOM | 7785 | ND1 | HIS | A 993 | 52.085 | 49.133 | −36.485 | 1.00 | 0.00 | N |
| ATOM | 7786 | CD2 | HIS | A 993 | 53.574 | 50.599 | −35.851 | 1.00 | 0.00 | C |
| ATOM | 7787 | CE1 | HIS | A 993 | 51.663 | 50.318 | −36.889 | 1.00 | 0.00 | C |
| ATOM | 7788 | NE2 | HIS | A 993 | 52.549 | 51.225 | −36.517 | 1.00 | 0.00 | N |
| ATOM | 7789 | N | THR | A 994 | 53.781 | 47.598 | −31.691 | 1.00 | 0.00 | N |
| ATOM | 7790 | CA | THR | A 994 | 54.044 | 48.237 | −30.404 | 1.00 | 0.00 | C |
| ATOM | 7791 | C | THR | A 994 | 55.011 | 47.331 | −29.644 | 1.00 | 0.00 | C |
| ATOM | 7792 | O | THR | A 994 | 54.918 | 46.104 | −29.726 | 1.00 | 0.00 | O |
| ATOM | 7793 | CB | THR | A 994 | 52.760 | 48.422 | −29.564 | 1.00 | 0.00 | C |
| ATOM | 7794 | OG1 | THR | A 994 | 52.154 | 47.147 | −29.321 | 1.00 | 0.00 | O |
| ATOM | 7795 | CG2 | THR | A 994 | 51.775 | 49.328 | −30.290 | 1.00 | 0.00 | C |
| ATOM | 7796 | N | GLN | A 995 | 55.938 | 47.935 | −28.911 | 1.00 | 0.00 | N |
| ATOM | 7797 | CA | GLN | A 995 | 56.930 | 47.175 | −28.164 | 1.00 | 0.00 | C |
| ATOM | 7798 | C | GLN | A 995 | 56.453 | 46.797 | −26.770 | 1.00 | 0.00 | C |
| ATOM | 7799 | O | GLN | A 995 | 55.673 | 47.517 | −26.147 | 1.00 | 0.00 | O |
| ATOM | 7800 | CB | GLN | A 995 | 58.215 | 47.985 | −28.033 | 1.00 | 0.00 | C |
| ATOM | 7801 | CG | GLN | A 995 | 58.870 | 48.646 | −29.315 | 1.00 | 0.00 | C |
| ATOM | 7802 | CD | GLN | A 995 | 59.787 | 49.635 | −29.072 | 1.00 | 0.00 | C |
| ATOM | 7803 | OE1 | GLN | A 995 | 60.900 | 49.255 | −28.700 | 1.00 | 0.00 | O |
| ATOM | 7804 | NE2 | GLN | A 995 | 59.493 | 50.915 | −29.266 | 1.00 | 0.00 | N |
| ATOM | 7805 | N | LYS | A 996 | 56.933 | 45.660 | −26.284 | 1.00 | 0.00 | N |
| ATOM | 7806 | CA | LYS | A 996 | 56.578 | 45.214 | −24.950 | 1.00 | 0.00 | C |
| ATOM | 7807 | C | LYS | A 996 | 57.287 | 46.147 | −23.979 | 1.00 | 0.00 | C |
| ATOM | 7808 | O | LYS | A 996 | 58.456 | 46.480 | −24.174 | 1.00 | 0.00 | O |
| ATOM | 7809 | CB | LYS | A 996 | 57.047 | 43.773 | −24.726 | 1.00 | 0.00 | C |
| ATOM | 7810 | CG | LYS | A 996 | 56.326 | 42.746 | −25.586 | 1.00 | 0.00 | C |
| ATOM | 7811 | CD | LYS | A 996 | 54.839 | 42.691 | −25.249 | 1.00 | 0.00 | C |
| ATOM | 7812 | CE | LYS | A 996 | 54.130 | 41.618 | −26.058 | 1.00 | 0.00 | C |
| ATOM | 7813 | NZ | LYS | A 996 | 54.698 | 40.267 | −25.786 | 1.00 | 0.00 | N |
| ATOM | 7814 | N | LEU | A 997 | 56.577 | 46.583 | −22.946 | 1.00 | 0.00 | N |
| ATOM | 7815 | CA | LEU | A 997 | 57.173 | 47.472 | −21.962 | 1.00 | 0.00 | C |
| ATOM | 7816 | C | LEU | A 997 | 57.565 | 46.708 | −20.710 | 1.00 | 0.00 | C |
| ATOM | 7817 | O | LEU | A 997 | 56.713 | 46.147 | −20.023 | 1.00 | 0.00 | O |
| ATOM | 7818 | CB | LEU | A 997 | 56.203 | 48.597 | −21.588 | 1.00 | 0.00 | C |
| ATOM | 7819 | CG | LEU | A 997 | 56.677 | 49.529 | −20.463 | 1.00 | 0.00 | C |
| ATOM | 7820 | CD1 | LEU | A 997 | 57.946 | 50.258 | −20.885 | 1.00 | 0.00 | C |
| ATOM | 7821 | CD2 | LEU | A 997 | 55.576 | 50.524 | −20.128 | 1.00 | 0.00 | C |
| ATOM | 7822 | N | ASP | A 998 | 58.863 | 46.679 | −20.432 | 1.00 | 0.00 | N |
| ATOM | 7823 | CA | ASP | A 998 | 59.390 | 46.015 | −19.247 | 1.00 | 0.00 | C |
| ATOM | 7824 | C | ASP | A 998 | 60.001 | 47.118 | −18.396 | 1.00 | 0.00 | C |
| ATOM | 7825 | O | ASP | A 998 | 61.173 | 47.458 | −18.555 | 1.00 | 0.00 | O |
| ATOM | 7826 | CB | ASP | A 998 | 60.468 | 44.998 | −19.633 | 1.00 | 0.00 | C |
| ATOM | 7827 | CG | ASP | A 998 | 61.140 | 44.373 | −18.423 | 1.00 | 0.00 | C |
| ATOM | 7828 | OD1 | ASP | A 998 | 62.150 | 43.661 | −18.609 | 1.00 | 0.00 | O |
| ATOM | 7829 | OD2 | ASP | A 998 | 60.658 | 44.589 | −17.288 | 1.00 | 0.00 | O |
| ATOM | 7830 | N | VAL | A 999 | 59.203 | 47.685 | −17.497 | 1.00 | 0.00 | N |
| ATOM | 7831 | CA | VAL | A 999 | 59.681 | 48.770 | −16.652 | 1.00 | 0.00 | C |
| ATOM | 7832 | C | VAL | A 999 | 60.915 | 48.416 | −15.831 | 1.00 | 0.00 | C |
| ATOM | 7833 | O | VAL | A 999 | 61.680 | 49.300 | −15.447 | 1.00 | 0.00 | O |
| ATOM | 7834 | CB | VAL | A 999 | 58.572 | 49.283 | −15.693 | 1.00 | 0.00 | C |
| ATOM | 7835 | CG1 | VAL | A 999 | 57.392 | 49.791 | −16.502 | 1.00 | 0.00 | C |
| ATOM | 7936 | CG2 | VAL | A 999 | 58.139 | 48.184 | −14.735 | 1.00 | 0.00 | C |
| ATOM | 7837 | N | CYS | A1000 | 61.126 | 47.134 | −15.563 | 1.00 | 0.00 | N |
| ATOM | 7838 | CA | CYS | A1000 | 62.289 | 46.765 | −14.774 | 1.00 | 0.00 | C |
| ATOM | 7839 | C | CYS | A1000 | 63.614 | 46.969 | −15.503 | 1.00 | 0.00 | C |
| ATOM | 7840 | O | CYS | A1000 | 64.668 | 46.992 | −14.878 | 1.00 | 0.00 | O |
| ATOM | 7841 | CB | CYS | A1000 | 62.156 | 45.332 | −14.260 | 1.00 | 0.00 | C |
| ATOM | 7842 | SG | CYS | A1000 | 61.384 | 45.278 | −12.604 | 1.00 | 0.00 | S |
| ATOM | 7843 | N | HIS | A1001 | 63.564 | 47.135 | −16.821 | 1.00 | 0.00 | N |
| ATOM | 7844 | CA | HIS | A1001 | 64.791 | 47.370 | −17.577 | 1.00 | 0.00 | C |
| ATOM | 7845 | C | HIS | A1001 | 64.900 | 48.799 | −18.109 | 1.00 | 0.00 | C |
| ATOM | 7846 | O | HIS | A1001 | 65.747 | 49.090 | −18.955 | 1.00 | 0.00 | O |
| ATOM | 7847 | CB | HIS | A1001 | 64.925 | 46.371 | −18.729 | 1.00 | 0.00 | C |
| ATOM | 7848 | CG | HIS | A1001 | 65.448 | 45.035 | −18.304 | 1.00 | 0.00 | C |
| ATOM | 7849 | ND1 | HIS | A1001 | 64.643 | 44.058 | −17.761 | 1.00 | 0.00 | N |
| ATOM | 7850 | CD2 | HIS | A1001 | 66.706 | 44.535 | −18.293 | 1.00 | 0.00 | C |
| ATOM | 7851 | CE1 | HIS | A1001 | 65.382 | 43.013 | −17.433 | 1.00 | 0.00 | C |
| ATOM | 7852 | NE2 | HIS | A1001 | 66.638 | 43.277 | −17.744 | 1.00 | 0.00 | N |
| ATOM | 7853 | N | LEU | A1002 | 64.047 | 49.692 | −17.614 | 1.00 | 0.00 | N |
| ATOM | 7854 | CA | LEU | A1002 | 64.086 | 51.088 | −18.045 | 1.00 | 0.00 | C |
| ATOM | 7855 | C | LEU | A1002 | 65.368 | 51.738 | −17.551 | 1.00 | 0.00 | C |
| ATOM | 7956 | O | LEU | A1002 | 65.914 | 52.628 | −18.202 | 1.00 | 0.00 | O |
| ATOM | 7857 | CB | LEU | A1002 | 62.871 | 51.855 | −17.519 | 1.00 | 0.00 | C |
| ATOM | 7858 | CG | LEU | A1002 | 61.593 | 51.676 | −18.339 | 1.00 | 0.00 | C |
| ATOM | 7859 | CD1 | LEU | A1002 | 60.439 | 52.392 | −17.658 | 1.00 | 0.00 | C |
| ATOM | 7860 | CD2 | LEU | A1002 | 61.810 | 52.221 | −19.745 | 1.00 | 0.00 | C |
| ATOM | 7861 | N | LEU | A1003 | 65.837 | 51.297 | −16.388 | 1.00 | 0.00 | N |
| ATOM | 7862 | CA | LEU | A1003 | 67.083 | 51.801 | −15.827 | 1.00 | 0.00 | C |
| ATOM | 7863 | C | LEU | A1003 | 68.108 | 50.676 | −15.939 | 1.00 | 0.00 | C |
| ATOM | 7864 | O | LEU | A1003 | 67.772 | 49.502 | −15.785 | 1.00 | 0.00 | O |
| ATOM | 7865 | CB | LEU | A1003 | 66.897 | 52.217 | −14.368 | 1.00 | 0.00 | C |
| ATOM | 7866 | CG | LEU | A1003 | 66.049 | 53.477 | −14.167 | 1.00 | 0.00 | C |
| ATOM | 7867 | CD1 | LEU | A1003 | 65.967 | 53.807 | −12.694 | 1.00 | 0.00 | C |
| ATOM | 7868 | CD2 | LEU | A1003 | 66.667 | 54.639 | −14.926 | 1.00 | 0.00 | C |
| ATOM | 7869 | N | PRO | A1004 | 69.373 | 51.022 | −16.214 | 1.00 | 0.00 | N |
| ATOM | 7870 | CA | PRO | A1004 | 70.442 | 50.030 | −16.357 | 1.00 | 0.00 | C |
| ATOM | 7871 | C | PRO | A1004 | 70.900 | 49.361 | −15.068 | 1.00 | 0.00 | C |
| ATOM | 7872 | O | PRO | A1004 | 70.573 | 49.804 | −13.962 | 1.00 | 0.00 | O |
| ATOM | 7873 | CB | PRO | A1004 | 71.560 | 50.839 | −17.002 | 1.00 | 0.00 | C |
| ATOM | 7874 | CG | PRO | A1004 | 71.410 | 52.161 | −16.320 | 1.00 | 0.00 | C |
| ATOM | 7875 | CD | PRO | A1004 | 69.906 | 52.387 | −16.384 | 1.00 | 0.00 | C |
| ATOM | 7876 | N | ASN | A1005 | 71.653 | 48.278 | −15.234 | 1.00 | 0.00 | N |
| ATOM | 7877 | CA | ASN | A1005 | 72.214 | 47.535 | −14.115 | 1.00 | 0.00 | C |
| ATOM | 7878 | C | ASN | A1005 | 71.172 | 46.948 | −13.170 | 1.00 | 0.00 | C |
| ATOM | 7879 | O | ASN | A1005 | 71.366 | 46.943 | −11.956 | 1.00 | 0.00 | O |
| ATOM | 7880 | CB | ASN | A1005 | 73.154 | 48.448 | −13.331 | 1.00 | 0.00 | C |
| ATOM | 7881 | CG | ASN | A1005 | 74.155 | 49.154 | −14.226 | 1.00 | 0.00 | C |
| ATOM | 7882 | OD1 | ASN | A1005 | 74.340 | 50.370 | −14.132 | 1.00 | 0.00 | O |
| ATOM | 7883 | ND2 | ASN | A1005 | 74.808 | 48.395 | −15.098 | 1.00 | 0.00 | N |
| ATOM | 7884 | N | VAL | A1006 | 70.073 | 46.447 | −13.720 | 1.00 | 0.00 | N |
| ATOM | 7885 | CA | VAL | A1006 | 69.029 | 45.864 | −12.885 | 1.00 | 0.00 | C |
| ATOM | 7886 | C | VAL | A1006 | 69.545 | 44.576 | −12.245 | 1.00 | 0.00 | C |
| ATOM | 7887 | O | VAL | A1006 | 70.105 | 43.714 | −12.923 | 1.00 | 0.00 | O |
| ATOM | 7888 | CB | VAL | A1006 | 67.745 | 45.570 | −13.701 | 1.00 | 0.00 | C |
| ATOM | 7889 | CG1 | VAL | A1006 | 68.040 | 44.593 | −14.822 | 1.00 | 0.00 | C |
| ATOM | 7890 | CG2 | VAL | A1006 | 66.660 | 45.020 | −12.782 | 1.00 | 0.00 | C |
| ATOM | 7891 | N | ALA | A1007 | 69.363 | 44.462 | −10.933 | 1.00 | 0.00 | N |
| ATOM | 7892 | CA | ALA | A1007 | 69.815 | 43.294 | −10.184 | 1.00 | 0.00 | C |
| ATOM | 7893 | C | ALA | A1007 | 68.642 | 42.398 | −9.790 | 1.00 | 0.00 | C |
| ATOM | 7894 | O | ALA | A1007 | 68.819 | 41.208 | −9.522 | 1.00 | 0.00 | O |
| ATOM | 7895 | CB | ALA | A1007 | 70.584 | 43.739 | −8.941 | 1.00 | 0.00 | C |
| ATOM | 7896 | N | ARG | A1008 | 67.446 | 42.974 | −9.739 | 1.00 | 0.00 | N |
| ATOM | 7897 | CA | ARG | A1008 | 66.252 | 42.204 | −9.412 | 1.00 | 0.00 | C |
| ATOM | 7898 | C | ARG | A1008 | 64.983 | 42.973 | −9.747 | 1.00 | 0.00 | C |
| ATOM | 7899 | O | ARG | A1008 | 64.980 | 44.205 | −9.807 | 1.00 | 0.00 | O |
| ATOM | 7900 | CB | ARG | A1008 | 66.240 | 41.784 | −7.936 | 1.00 | 0.00 | C |
| ATOM | 7901 | CG | ARG | A1008 | 66.187 | 42.913 | −6.931 | 1.00 | 0.00 | C |
| ATOM | 7902 | CD | ARG | A1008 | 66.059 | 42.362 | −5.511 | 1.00 | 0.00 | C |
| ATOM | 7903 | NE | ARG | A1008 | 67.201 | 41.527 | −5.135 | 1.00 | 0.00 | N |
| ATOM | 7904 | CZ | ARG | A1008 | 68.451 | 41.970 | −5.012 | 1.00 | 0.00 | C |
| ATOM | 7905 | NH1 | ARG | A1008 | 68.735 | 43.248 | −5.232 | 1.00 | 0.00 | N |
| ATOM | 7906 | NH2 | ARG | A1008 | 69.422 | 41.130 | −4.673 | 1.00 | 0.00 | N |
| ATOM | 7907 | N | CYS | A1009 | 63.911 | 42.225 | −9.980 | 1.00 | 0.00 | N |
| ATOM | 7908 | CA | CYS | A1009 | 62.618 | 42.797 | −10.323 | 1.00 | 0.00 | C |
| ATOM | 7909 | C | CYS | A1009 | 61.573 | 42.098 | −9.473 | 1.00 | 0.00 | C |
| ATOM | 7910 | O | CYS | A1009 | 61.503 | 40.867 | −9.454 | 1.00 | 0.00 | O |
| ATOM | 7911 | CB | CYS | A1009 | 62.323 | 42.562 | −11.800 | 1.00 | 0.00 | C |
| ATOM | 7912 | SG | CYS | A1009 | 60.805 | 43.356 | −12.418 | 1.00 | 0.00 | S |
| ATOM | 7913 | N | GLU | A1010 | 60.757 | 42.879 | −8.774 | 1.00 | 0.00 | N |
| ATOM | 7914 | CA | GLU | A1010 | 59.741 | 42.304 | −7.909 | 1.00 | 0.00 | C |
| ATOM | 7915 | C | GLU | A1010 | 58.370 | 42.940 | −8.094 | 1.00 | 0.00 | C |
| ATOM | 7916 | O | GLU | A1010 | 58.255 | 44.144 | −8.343 | 1.00 | 0.00 | O |
| ATOM | 7917 | CB | GLU | A1010 | 60.177 | 42.454 | −6.448 | 1.00 | 0.00 | C |
| ATOM | 7918 | CG | GLU | A1010 | 61.490 | 41.749 | −6.118 | 1.00 | 0.00 | C |
| ATOM | 7919 | CD | GLU | A1010 | 62.115 | 42.239 | −4.826 | 1.00 | 0.00 | C |
| ATOM | 7920 | OE1 | GLU | A1010 | 62.535 | 43.417 | −4.778 | 1.00 | 0.00 | O |
| ATOM | 7921 | OE2 | GLU | A1010 | 62.187 | 41.449 | −3.858 | 1.00 | 0.00 | O |
| ATOM | 7922 | N | ARG | A1011 | 57.331 | 42.119 | −7.990 | 1.00 | 0.00 | N |
| ATOM | 7923 | CA | ARG | A1011 | 55.968 | 42.619 | −8.082 | 1.00 | 0.00 | C |
| ATOM | 7924 | C | ARG | A1011 | 55.648 | 42.973 | −6.634 | 1.00 | 0.00 | C |
| ATOM | 7925 | O | ARG | A1011 | 55.974 | 42.213 | −5.720 | 1.00 | 0.00 | O |
| ATOM | 7926 | CB | ARG | A1011 | 55.009 | 41.535 | −8.574 | 1.00 | 0.00 | C |
| ATOM | 7927 | CG | ARG | A1011 | 53.594 | 42.048 | −8.837 | 1.00 | 0.00 | C |
| ATOM | 7928 | CD | ARG | A1011 | 52.583 | 40.913 | −8.851 | 1.00 | 0.00 | C |
| ATOM | 7929 | NE | ARG | A1011 | 52.917 | 39.880 | −9.826 | 1.00 | 0.00 | N |
| ATOM | 7930 | CZ | ARG | A1011 | 52.897 | 40.055 | −11.144 | 1.00 | 0.00 | C |
| ATOM | 7931 | NH1 | ARG | A1011 | 52.556 | 41.233 | −11.654 | 1.00 | 0.00 | N |
| ATOM | 7932 | NH2 | ARG | A1011 | 53.215 | 39.051 | −11.953 | 1.00 | 0.00 | N |
| ATOM | 7933 | N | THR | A1012 | 55.027 | 44.125 | −6.419 | 1.00 | 0.00 | N |
| ATOM | 7934 | CA | THR | A1012 | 54.704 | 44.560 | −5.068 | 1.00 | 0.00 | C |
| ATOM | 7935 | C | THR | A1012 | 53.271 | 45.056 | −4.984 | 1.00 | 0.00 | C |
| ATOM | 7936 | O | THR | A1012 | 52.569 | 45.156 | −5.990 | 1.00 | 0.00 | O |
| ATOM | 7937 | CB | THR | A1012 | 55.600 | 45.741 | −4.630 | 1.00 | 0.00 | C |
| ATOM | 7938 | OG1 | THR | A1012 | 55.243 | 46.907 | −5.389 | 1.00 | 0.00 | O |
| ATOM | 7939 | CG2 | THR | A1012 | 57.072 | 45.429 | −4.865 | 1.00 | 0.00 | C |
| ATOM | 7940 | N | THR | A1013 | 52.842 | 45.362 | −3.767 | 1.00 | 0.00 | N |
| ATOM | 7941 | CA | THR | A1013 | 51.521 | 45.921 | −3.557 | 1.00 | 0.00 | C |
| ATOM | 7942 | C | THR | A1013 | 51.618 | 47.310 | −4.206 | 1.00 | 0.00 | C |
| ATOM | 7943 | O | THR | A1013 | 52.724 | 47.817 | −4.421 | 1.00 | 0.00 | O |
| ATOM | 7944 | CB | THR | A1013 | 51.225 | 46.037 | −2.050 | 1.00 | 0.00 | C |
| ATOM | 7945 | OG1 | THR | A1013 | 52.407 | 46.479 | −1.366 | 1.00 | 0.00 | O |
| ATOM | 7946 | CG2 | THR | A1013 | 50.807 | 44.674 | −1.481 | 1.00 | 0.00 | C |
| ATOM | 7947 | N | LEU | A1014 | 50.482 | 47.924 | −4.521 | 1.00 | 0.00 | N |
| ATOM | 7948 | CA | LEU | A1014 | 50.487 | 49.233 | −5.179 | 1.00 | 0.00 | C |
| ATOM | 7949 | C | LEU | A1014 | 51.176 | 50.344 | −4.403 | 1.00 | 0.00 | C |
| ATOM | 7950 | O | LEU | A1014 | 51.532 | 51.376 | −4.977 | 1.00 | 0.00 | O |
| ATOM | 7951 | CB | LEU | A1014 | 49.059 | 49.663 | −5.512 | 1.00 | 0.00 | C |
| ATOM | 7952 | CG | LEU | A1014 | 48.272 | 48.715 | −6.417 | 1.00 | 0.00 | C |
| ATOM | 7953 | CD1 | LEU | A1014 | 46.969 | 49.396 | −6.821 | 1.00 | 0.00 | C |
| ATOM | 7954 | CD2 | LEU | A1014 | 49.094 | 48.355 | −7.652 | 1.00 | 0.00 | C |
| ATOM | 7955 | N | THR | A1015 | 51.365 | 50.131 | −3.104 | 1.00 | 0.00 | N |
| ATOM | 7956 | CA | THR | A1015 | 52.021 | 51.104 | −2.235 | 1.00 | 0.00 | C |
| ATOM | 7957 | C | THR | A1015 | 53.529 | 50.863 | −2.184 | 1.00 | 0.00 | C |
| ATOM | 7958 | O | THR | A1015 | 54.264 | 51.630 | −1.550 | 1.00 | 0.00 | O |
| ATOM | 7959 | CB | THR | A1015 | 51.499 | 51.001 | −0.795 | 1.00 | 0.00 | C |
| ATOM | 7960 | OG1 | THR | A1015 | 51.618 | 49.642 | −0.353 | 1.00 | 0.00 | O |
| ATOM | 7961 | CG2 | THR | A1015 | 50.042 | 51.449 | −0.708 | 1.00 | 0.00 | C |
| ATOM | 7962 | N | PHE | A1016 | 53.972 | 49.792 | −2.844 | 1.00 | 0.00 | N |
| ATOM | 7963 | CA | PHE | A1016 | 55.387 | 49.406 | −2.891 | 1.00 | 0.00 | C |
| ATOM | 7964 | C | PHE | A1016 | 55.884 | 48.877 | −1.547 | 1.00 | 0.00 | C |
| ATOM | 7965 | O | PHE | A1016 | 57.071 | 48.584 | −1.394 | 1.00 | 0.00 | O |
| ATOM | 7966 | CB | PHE | A1015 | 56.270 | 50.602 | −3.283 | 1.00 | 0.00 | C |
| ATOM | 7967 | CG | PHE | A1016 | 55.967 | 51.177 | −4.638 | 1.00 | 0.00 | C |
| ATOM | 7968 | CD1 | PHE | A1016 | 55.825 | 52.553 | −4.800 | 1.00 | 0.00 | C |
| ATOM | 7969 | CD2 | PHE | A1016 | 55.837 | 50.355 | −5.750 | 1.00 | 0.00 | C |
| ATOM | 7970 | CE1 | PHE | A1016 | 55.556 | 53.101 | −6.052 | 1.00 | 0.00 | C |
| ATOM | 7971 | CE2 | PHE | A1016 | 55.568 | 50.896 | −7.010 | 1.00 | 0.00 | C |
| ATOM | 7972 | CZ | PHE | A1016 | 55.427 | 52.269 | −7.158 | 1.00 | 0.00 | C |
| ATOM | 7973 | N | LEU | A1017 | 54.982 | 48.734 | −0.580 | 1.00 | 0.00 | N |
| ATOM | 7974 | CA | LEU | A1017 | 55.378 | 48.292 | 0.754 | 1.00 | 0.00 | C |
| ATOM | 7975 | C | LEU | A1017 | 55.600 | 46.800 | 0.987 | 1.00 | 0.00 | C |
| ATOM | 7976 | O | LEU | A1017 | 56.282 | 46.425 | 1.941 | 1.00 | 0.00 | O |
| ATOM | 7977 | CB | LEU | A1017 | 54.388 | 48.849 | 1.785 | 1.00 | 0.00 | C |
| ATOM | 7978 | CG | LEU | A1017 | 54.346 | 50.389 | 1.783 | 1.00 | 0.00 | C |
| ATOM | 7979 | CD1 | LEU | A1017 | 53.334 | 50.891 | 2.800 | 1.00 | 0.00 | C |
| ATOM | 7980 | CD2 | LEU | A1017 | 55.733 | 50.947 | 2.097 | 1.00 | 0.00 | C |
| ATOM | 7981 | N | GLN | A1018 | 55.039 | 45.945 | 0.139 | 1.00 | 0.00 | N |
| ATOM | 7982 | CA | GLN | A1018 | 55.241 | 44.508 | 0.308 | 1.00 | 0.00 | C |
| ATOM | 7983 | C | GLN | A1018 | 55.602 | 43.819 | −1.005 | 1.00 | 0.00 | C |
| ATOM | 7984 | O | GLN | A1018 | 54.967 | 44.049 | −2.034 | 1.00 | 0.00 | O |
| ATOM | 7985 | CB | GLN | A1018 | 53.989 | 43.837 | 0.890 | 1.00 | 0.00 | C |
| ATOM | 7986 | CG | GLN | A1018 | 54.178 | 42.329 | 1.116 | 1.00 | 0.00 | C |
| ATOM | 7987 | CD | GLN | A1018 | 52.897 | 41.591 | 1.474 | 1.00 | 0.00 | C |
| ATOM | 7988 | OE1 | GLN | A1018 | 52.911 | 40.375 | 1.688 | 1.00 | 0.00 | O |
| ATOM | 7989 | NE2 | GLN | A1018 | 51.784 | 42.315 | 1.534 | 1.00 | 0.00 | N |
| ATOM | 7990 | N | ASN | A1019 | 56.624 | 42.969 | −0.967 | 1.00 | 0.00 | N |
| ATOM | 7991 | CA | ASN | A1019 | 57.032 | 42.237 | −2.158 | 1.00 | 0.00 | C |
| ATOM | 7992 | C | ASN | A1019 | 56.123 | 41.021 | −2.288 | 1.00 | 0.00 | C |
| ATOM | 7993 | O | ASN | A1019 | 55.946 | 40.266 | −1.333 | 1.00 | 0.00 | O |
| ATOM | 7994 | CB | ASN | A1019 | 58.494 | 41.795 | −2.046 | 1.00 | 0.00 | C |
| ATOM | 7995 | CG | ASN | A1019 | 59.445 | 42.967 | −1.889 | 1.00 | 0.00 | C |
| ATOM | 7996 | OD1 | ASN | A1019 | 59.319 | 43.980 | −2.583 | 1.00 | 0.00 | O |
| ATOM | 7997 | ND2 | ASN | A1019 | 60.412 | 42.833 | −0.985 | 1.00 | 0.00 | N |
| ATOM | 7998 | N | LEU | A1020 | 55.542 | 40.837 | −3.468 | 1.00 | 0.00 | N |
| ATOM | 7999 | CA | LEU | A1020 | 54.633 | 39.724 | −3.706 | 1.00 | 0.00 | C |
| ATOM | 8000 | C | LEU | A1020 | 55.233 | 38.645 | −4.597 | 1.00 | 0.00 | C |
| ATOM | 8001 | O | LEU | A1020 | 54.877 | 37.470 | −4.480 | 1.00 | 0.00 | O |
| ATOM | 8002 | CB | LEU | A1020 | 53.340 | 40.230 | −4.351 | 1.00 | 0.00 | C |
| ATOM | 8003 | CG | LEU | A1020 | 52.538 | 41.316 | −3.629 | 1.00 | 0.00 | C |
| ATOM | 8004 | CD1 | LEU | A1020 | 51.419 | 41.807 | −4.540 | 1.00 | 0.00 | C |
| ATOM | 8005 | CD2 | LEU | A1020 | 51.974 | 40.771 | −2.327 | 1.00 | 0.00 | C |
| ATOM | 8006 | N | GLU | A1021 | 56.134 | 39.040 | −5.492 | 1.00 | 0.00 | N |
| ATOM | 8007 | CA | GLU | A1021 | 56.750 | 38.088 | −6.414 | 1.00 | 0.00 | C |
| ATOM | 8008 | C | GLU | A1021 | 58.165 | 38.439 | −6.848 | 1.00 | 0.00 | C |
| ATOM | 8009 | O | GLU | A1021 | 58.472 | 39.599 | −7.125 | 1.00 | 0.00 | O |
| ATOM | 8010 | CB | GLU | A1021 | 55.910 | 37.959 | −7.686 | 1.00 | 0.00 | C |
| ATOM | 8011 | CG | GLU | A1021 | 54.585 | 37.250 | −7.547 | 1.00 | 0.00 | C |
| ATOM | 8012 | CD | GLU | A1021 | 53.900 | 37.083 | −8.893 | 1.00 | 0.00 | C |
| ATOM | 8013 | OE1 | GLU | A1021 | 54.566 | 36.609 | −9.842 | 1.00 | 0.00 | O |
| ATOM | 8014 | OE2 | GLU | A1021 | 52.701 | 37.421 | −9.005 | 1.00 | 0.00 | O |
| ATOM | 8015 | N | HIS | A1022 | 59.017 | 37.420 | −6.917 | 1.00 | 0.00 | N |
| ATOM | 8016 | CA | HIS | A1022 | 60.388 | 37.590 | −7.379 | 1.00 | 0.00 | C |
| ATOM | 8017 | C | HIS | A1022 | 60.300 | 37.187 | −8.848 | 1.00 | 0.00 | C |
| ATOM | 8018 | O | HIS | A1022 | 60.066 | 36.020 | −9.170 | 1.00 | 0.00 | O |
| ATOM | 8019 | CB | HIS | A1022 | 61.338 | 36.660 | −6.622 | 1.00 | 0.00 | C |
| ATOM | 8020 | CG | HIS | A1022 | 62.776 | 36.828 | −7.005 | 1.00 | 0.00 | C |
| ATOM | 8021 | ND1 | HIS | A1022 | 63.436 | 38.034 | −6.906 | 1.00 | 0.00 | N |
| ATOM | 8022 | CD2 | HIS | A1022 | 63.679 | 35.945 | −7.494 | 1.00 | 0.00 | C |
| ATOM | 8023 | CE1 | HIS | A1022 | 64.683 | 37.887 | −7.317 | 1.00 | 0.00 | C |
| ATOM | 8024 | NE2 | HIS | A1022 | 64.857 | 36.628 | −7.679 | 1.00 | 0.00 | N |
| ATOM | 8025 | N | LEU | A1023 | 60.474 | 38.160 | −9.733 | 1.00 | 0.00 | N |
| ATOM | 8026 | CA | LEU | A1023 | 60.357 | 37.928 | −11.166 | 1.00 | 0.00 | C |
| ATOM | 8027 | C | LEU | A1023 | 61.607 | 37.393 | −11.851 | 1.00 | 0.00 | C |
| ATOM | 8028 | O | LEU | A1023 | 62.663 | 38.024 | −11.828 | 1.00 | 0.00 | O |
| ATOM | 8029 | CB | LEU | A1023 | 59.911 | 39.225 | −11.841 | 1.00 | 0.00 | C |
| ATOM | 8030 | CG | LEU | A1023 | 58.658 | 39.827 | −11.195 | 1.00 | 0.00 | C |
| ATOM | 8031 | CD1 | LEU | A1023 | 58.383 | 41.203 | −11.767 | 1.00 | 0.00 | C |
| ATOM | 8032 | CD2 | LEU | A1023 | 57.477 | 38.896 | −11.420 | 1.00 | 0.00 | C |
| ATOM | 8033 | N | ASP | A1024 | 61.473 | 36.224 | −12.469 | 1.00 | 0.00 | N |
| ATOM | 8034 | CA | ASP | A1024 | 62.587 | 35.603 | −13.174 | 1.00 | 0.00 | C |
| ATOM | 8035 | C | ASP | A1024 | 63.002 | 36.444 | −14.371 | 1.00 | 0.00 | C |
| ATOM | 8036 | O | ASP | A1024 | 62.179 | 37.137 | −14.972 | 1.00 | 0.00 | O |
| ATOM | 8037 | CB | ASP | A1024 | 62.207 | 34.194 | −13.638 | 1.00 | 0.00 | C |
| ATOM | 8038 | CG | ASP | A1024 | 62.364 | 33.154 | −12.540 | 1.00 | 0.00 | C |
| ATOM | 8039 | OD1 | ASP | A1024 | 62.094 | 31.964 | −12.807 | 1.00 | 0.00 | O |
| ATOM | 8040 | OD2 | ASP | A1024 | 62.763 | 33.524 | −11.413 | 1.00 | 0.00 | O |
| ATOM | 8041 | N | GLY | A1025 | 64.285 | 36.384 | −14.711 | 1.00 | 0.00 | N |
| ATOM | 8042 | CA | GLY | A1025 | 64.787 | 37.154 | −15.833 | 1.00 | 0.00 | C |
| ATOM | 8043 | C | GLY | A1025 | 64.712 | 38.640 | −15.551 | 1.00 | 0.00 | C |
| ATOM | 8044 | O | GLY | A1025 | 65.052 | 39.463 | −16.401 | 1.00 | 0.00 | O |
| ATOM | 8045 | N | MET | A1026 | 64.265 | 38.981 | −14.345 | 1.00 | 0.00 | N |
| ATOM | 8046 | CA | MET | A1026 | 64.139 | 40.372 | −13.935 | 1.00 | 0.00 | C |
| ATOM | 8047 | C | MET | A1026 | 63.255 | 41.126 | −14.921 | 1.00 | 0.00 | C |
| ATOM | 8048 | O | MET | A1026 | 63.495 | 42.295 | −15.220 | 1.00 | 0.00 | O |
| ATOM | 8049 | CB | MET | A1026 | 65.518 | 41.028 | −13.867 | 1.00 | 0.00 | C |
| ATOM | 8050 | CG | MET | A1026 | 66.519 | 40.268 | −13.015 | 1.00 | 0.00 | C |
| ATOM | 8051 | SD | MET | A1026 | 68.104 | 41.110 | −12.896 | 1.00 | 0.00 | S |
| ATOM | 8052 | CE | MET | A1026 | 68.904 | 40.531 | −14.390 | 1.00 | 0.00 | C |
| ATOM | 8053 | N | VAL | A1027 | 62.231 | 40.447 | −15.425 | 1.00 | 0.00 | N |
| ATOM | 8054 | CA | VAL | A1027 | 61.318 | 41.058 | −16.380 | 1.00 | 0.00 | C |
| ATOM | 8055 | C | VAL | A1027 | 59.932 | 41.268 | −15.791 | 1.00 | 0.00 | C |
| ATOM | 8056 | O | VAL | A1027 | 59.299 | 40.328 | −15.311 | 1.00 | 0.00 | O |
| ATOM | 8057 | CB | VAL | A1027 | 61.177 | 40.199 | −17.651 | 1.00 | 0.00 | C |
| ATOM | 8058 | CG1 | VAL | A1027 | 60.154 | 40.826 | −18.590 | 1.00 | 0.00 | C |
| ATOM | 8059 | CG2 | VAL | A1027 | 62.522 | 40.072 | −18.343 | 1.00 | 0.00 | C |
| ATOM | 8060 | N | ALA | A1028 | 59.464 | 42.510 | −15.833 | 1.00 | 0.00 | N |
| ATOM | 8061 | CA | ALA | A1028 | 58.142 | 42.838 | −15.322 | 1.00 | 0.00 | C |
| ATOM | 8062 | C | ALA | A1028 | 57.147 | 42.690 | −16.463 | 1.00 | 0.00 | C |
| ATOM | 8063 | O | ALA | A1028 | 57.124 | 43.502 | −17.390 | 1.00 | 0.00 | O |
| ATOM | 8064 | CB | ALA | A1028 | 58.118 | 44.266 | −14.782 | 1.00 | 0.00 | C |
| ATOM | 8065 | N | PRO | A1029 | 56.319 | 41.639 | −16.419 | 1.00 | 0.00 | N |
| ATOM | 8066 | CA | PRO | A1029 | 55.322 | 41.404 | −17.466 | 1.00 | 0.00 | C |
| ATOM | 8067 | C | PRO | A1029 | 54.213 | 42.453 | −17.411 | 1.00 | 0.00 | C |
| ATOM | 8068 | O | PRO | A1029 | 53.915 | 42.994 | −16.347 | 1.00 | 0.00 | O |
| ATOM | 8069 | CB | PRO | A1029 | 54.814 | 40.005 | −17.142 | 1.00 | 0.00 | C |
| ATOM | 8070 | CG | PRO | A1029 | 54.879 | 39.978 | −15.649 | 1.00 | 0.00 | C |
| ATOM | 8071 | CD | PRO | A1029 | 56.224 | 40.614 | −15.363 | 1.00 | 0.00 | C |
| ATOM | 8072 | N | GLU | A1030 | 53.608 | 42.743 | −18.557 | 1.00 | 0.00 | N |
| ATOM | 8073 | CA | GLU | A1030 | 52.536 | 43.726 | −18.607 | 1.00 | 0.00 | C |
| ATOM | 8074 | C | GLU | A1030 | 51.298 | 43.154 | −17.924 | 1.00 | 0.00 | C |
| ATOM | 8075 | O | GLU | A1030 | 51.187 | 41.940 | −17.728 | 1.00 | 0.00 | O |
| ATOM | 8076 | CB | GLU | A1030 | 52.221 | 44.095 | −20.059 | 1.00 | 0.00 | C |
| ATOM | 8077 | CG | GLU | A1030 | 53.444 | 44.504 | −20.871 | 1.00 | 0.00 | C |
| ATOM | 8078 | CD | GLU | A1030 | 53.085 | 44.988 | −22.264 | 1.00 | 0.00 | C |
| ATOM | 8079 | OE1 | GLU | A1030 | 52.163 | 44.407 | −22.872 | 1.00 | 0.00 | O |
| ATOM | 8080 | OE2 | GLU | A1030 | 53.728 | 45.940 | −22.755 | 1.00 | 0.00 | O |
| ATOM | 8081 | N | VAL | A1031 | 50.366 | 44.029 | −17.564 | 1.00 | 0.00 | N |
| ATOM | 8082 | CA | VAL | A1031 | 49.155 | 43.599 | −16.885 | 1.00 | 0.00 | C |
| ATOM | 8083 | C | VAL | A1031 | 47.976 | 43.392 | −17.825 | 1.00 | 0.00 | C |
| ATOM | 8084 | O | VAL | A1031 | 48.024 | 43.762 | −18.995 | 1.00 | 0.00 | O |
| ATOM | 8085 | CB | VAL | A1031 | 48.752 | 44.609 | −15.791 | 1.00 | 0.00 | C |
| ATOM | 8086 | CG1 | VAL | A1031 | 49.863 | 44.713 | −14.758 | 1.00 | 0.00 | C |
| ATOM | 8087 | CG2 | VAL | A1031 | 48.471 | 45.965 | −16.406 | 1.00 | 0.00 | C |
| ATOM | 8088 | N | CYS | A1032 | 46.921 | 42.786 | −17.291 | 1.00 | 0.00 | N |
| ATOM | 8089 | CA | CYS | A1032 | 45.705 | 42.505 | −18.044 | 1.00 | 0.00 | C |
| ATOM | 8090 | C | CYS | A1032 | 44.805 | 43.735 | −18.126 | 1.00 | 0.00 | C |
| ATOM | 8091 | O | CYS | A1032 | 44.983 | 44.694 | −17.377 | 1.00 | 0.00 | O |
| ATOM | 8092 | CB | CYS | A1032 | 44.927 | 41.372 | −17.370 | 1.00 | 0.00 | C |
| ATOM | 8093 | SG | CYS | A1032 | 45.687 | 39.716 | −17.446 | 1.00 | 0.00 | S |
| ATOM | 8094 | N | PRO | A1033 | 43.822 | 43.720 | −19.044 | 1.00 | 0.00 | N |
| ATOM | 8095 | CA | PRO | A1033 | 42.907 | 44.857 | −19.181 | 1.00 | 0.00 | C |
| ATOM | 8096 | C | PRO | A1033 | 42.190 | 45.112 | −17.852 | 1.00 | 0.00 | C |
| ATOM | 8097 | O | PRO | A1033 | 41.719 | 44.176 | −17.205 | 1.00 | 0.00 | O |
| ATOM | 8098 | CB | PRO | A1033 | 41.944 | 44.393 | −20.272 | 1.00 | 0.00 | C |
| ATOM | 8099 | CG | PRO | A1033 | 42.818 | 43.522 | −21.135 | 1.00 | 0.00 | C |
| ATOM | 8100 | CD | PRO | A1033 | 43.575 | 42.720 | −20.100 | 1.00 | 0.00 | C |
| ATOM | 8101 | N | MET | A1034 | 42.118 | 46.381 | −17.464 | 1.00 | 0.00 | N |
| ATOM | 8102 | CA | MET | A1034 | 41.479 | 46.814 | −16.225 | 1.00 | 0.00 | C |
| ATOM | 8103 | C | MET | A1034 | 42.244 | 46.394 | −14.974 | 1.00 | 0.00 | C |
| ATOM | 8104 | O | MET | A1034 | 41.728 | 46.497 | −13.861 | 1.00 | 0.00 | O |
| ATOM | 8105 | CB | MET | A1034 | 40.039 | 46.305 | −16.146 | 1.00 | 0.00 | C |
| ATOM | 8106 | CG | MET | A1034 | 39.127 | 46.880 | −17.219 | 1.00 | 0.00 | C |
| ATOM | 8107 | SD | MET | A1034 | 39.211 | 48.686 | −17.324 | 1.00 | 0.00 | S |
| ATOM | 8108 | CE | MET | A1034 | 38.245 | 49.175 | −15.870 | 1.00 | 0.00 | C |
| ATOM | 8109 | N | GLU | A1035 | 43.473 | 45.926 | −15.163 | 1.00 | 0.00 | N |
| ATOM | 8110 | CA | GLU | A1035 | 44.312 | 45.516 | −14.045 | 1.00 | 0.00 | C |
| ATOM | 8111 | C | GLU | A1035 | 45.381 | 46.568 | −13.765 | 1.00 | 0.00 | C |
| ATOM | 8112 | O | GLU | A1035 | 45.774 | 47.325 | −14.652 | 1.00 | 0.00 | O |
| ATOM | 8113 | CB | GLU | A1035 | 44.984 | 44.165 | −14.337 | 1.00 | 0.00 | C |
| ATOM | 8114 | CG | GLU | A1035 | 46.199 | 43.874 | −13.451 | 1.00 | 0.00 | C |
| ATOM | 8115 | CD | GLU | A1035 | 46.687 | 42.430 | −13.531 | 1.00 | 0.00 | C |
| ATOM | 8116 | OE1 | GLU | A1035 | 46.956 | 41.933 | −14.651 | 1.00 | 0.00 | O |
| ATOM | 8117 | OE2 | GLU | A1035 | 46.812 | 41.795 | −12.459 | 1.00 | 0.00 | O |
| ATOM | 8118 | N | THR | A1036 | 45.833 | 46.618 | −12.518 | 1.00 | 0.00 | N |
| ATOM | 8119 | CA | THR | A1036 | 46.876 | 47.550 | −12.116 | 1.00 | 0.00 | C |
| ATOM | 8120 | C | THR | A1036 | 47.871 | 46.764 | −11.279 | 1.00 | 0.00 | C |
| ATOM | 8121 | O | THR | A1036 | 47.482 | 46.020 | −10.380 | 1.00 | 0.00 | O |
| ATOM | 8122 | CB | THR | A1036 | 46.320 | 48.701 | −11.252 | 1.00 | 0.00 | C |
| ATOM | 8123 | OG1 | THR | A1036 | 45.273 | 49.372 | −11.960 | 1.00 | 0.00 | O |
| ATOM | 8124 | CG2 | THR | A1036 | 47.421 | 49.702 | −10.926 | 1.00 | 0.00 | C |
| ATOM | 8125 | N | ALA | A1037 | 49.154 | 46.916 | −11.579 | 1.00 | 0.00 | N |
| ATOM | 8126 | CA | ALA | A1037 | 50.181 | 46.219 | −10.825 | 1.00 | 0.00 | C |
| ATOM | 8127 | C | ALA | A1037 | 51.314 | 47.177 | −10.521 | 1.00 | 0.00 | C |
| ATOM | 8128 | O | ALA | A1037 | 51.432 | 48.235 | −11.142 | 1.00 | 0.00 | O |
| ATOM | 8129 | CB | ALA | A1037 | 50.702 | 45.025 | −11.616 | 1.00 | 0.00 | C |
| ATOM | 8130 | N | ALA | A1038 | 52.132 | 46.815 | −9.544 | 1.00 | 0.00 | N |
| ATOM | 8131 | CA | ALA | A1038 | 53.269 | 47.636 | −9.166 | 1.00 | 0.00 | C |
| ATOM | 8132 | C | ALA | A1038 | 54.512 | 46.766 | −9.194 | 1.00 | 0.00 | C |
| ATOM | 8133 | O | ALA | A1038 | 54.467 | 45.589 | −8.824 | 1.00 | 0.00 | O |
| ATOM | 8134 | CB | ALA | A1038 | 53.068 | 48.213 | −7.776 | 1.00 | 0.00 | C |
| ATOM | 8135 | N | TYR | A1039 | 55.613 | 47.347 | −9.655 | 1.00 | 0.00 | N |
| ATOM | 8136 | CA | TYR | A1039 | 56.879 | 46.640 | −9.720 | 1.00 | 0.00 | C |
| ATOM | 8137 | C | TYR | A1039 | 57.977 | 47.547 | −9.204 | 1.00 | 0.00 | C |
| ATOM | 8138 | O | TYR | A1039 | 57.920 | 48.768 | −9.362 | 1.00 | 0.00 | O |
| ATOM | 8139 | CB | TYR | A1039 | 57.219 | 46.238 | −11.156 | 1.00 | 0.00 | C |
| ATOM | 8140 | CG | TYR | A1039 | 56.189 | 45.365 | −11.834 | 1.00 | 0.00 | C |
| ATOM | 8141 | CD1 | TYR | A1039 | 55.253 | 45.912 | −12.708 | 1.00 | 0.00 | C |
| ATOM | 8142 | CD2 | TYR | A1039 | 56.160 | 43.988 | −11.613 | 1.00 | 0.00 | C |
| ATOM | 8143 | CE1 | TYR | A1039 | 54.314 | 45.111 | −13.354 | 1.00 | 0.00 | C |
| ATOM | 8144 | CE2 | TYR | A1039 | 55.223 | 43.177 | −12.253 | 1.00 | 0.00 | C |
| ATOM | 8145 | CZ | TYR | A1039 | 54.306 | 43.745 | −13.123 | 1.00 | 0.00 | C |
| ATOM | 8146 | OH | TYR | A1039 | 53.385 | 42.950 | −13.768 | 1.00 | 0.00 | O |
| ATOM | 8147 | N | VAL | A1040 | 58.978 | 46.940 | −8.583 | 1.00 | 0.00 | N |
| ATOM | 8148 | CA | VAL | A1040 | 60.115 | 47.678 | −8.067 | 1.00 | 0.00 | C |
| ATOM | 8149 | C | VAL | A1040 | 61.359 | 47.015 | −8.637 | 1.00 | 0.00 | C |
| ATOM | 8150 | O | VAL | A1040 | 61.542 | 45.801 | −8.510 | 1.00 | 0.00 | O |
| ATOM | 8151 | CB | VAL | A1040 | 60.175 | 47.635 | −6.524 | 1.00 | 0.00 | C |
| ATOM | 8152 | CG1 | VAL | A1040 | 61.463 | 48.299 | −6.030 | 1.00 | 0.00 | C |
| ATOM | 8153 | CG2 | VAL | A1040 | 58.966 | 48.352 | −5.939 | 1.00 | 0.00 | C |
| ATOM | 8154 | N | SER | A1041 | 62.197 | 47.808 | −9.294 | 1.00 | 0.00 | N |
| ATOM | 8155 | CA | SER | A1041 | 63.431 | 47.288 | −9.862 | 1.00 | 0.00 | C |
| ATOM | 8156 | C | SER | A1041 | 64.585 | 47.760 | −8.987 | 1.00 | 0.00 | C |
| ATOM | 8157 | O | SER | A1041 | 64.615 | 48.915 | −8.564 | 1.00 | 0.00 | O |
| ATOM | 8158 | CB | SER | A1041 | 63.618 | 47.788 | −11.301 | 1.00 | 0.00 | C |
| ATOM | 8159 | OG | SER | A1041 | 63.698 | 49.202 | −11.355 | 1.00 | 0.00 | O |
| ATOM | 8160 | N | SER | A1042 | 65.521 | 46.856 | −8.707 | 1.00 | 0.00 | N |
| ATOM | 8161 | CA | SER | A1042 | 66.690 | 47.175 | −7.889 | 1.00 | 0.00 | C |
| ATOM | 8162 | C | SER | A1042 | 67.906 | 47.240 | −8.802 | 1.00 | 0.00 | C |
| ATOM | 8163 | O | SER | A1042 | 68.058 | 46.417 | −9.704 | 1.00 | 0.00 | O |
| ATOM | 8164 | CB | SER | A1042 | 66.895 | 46.111 | −6.809 | 1.00 | 0.00 | C |
| ATOM | 8165 | OG | SER | A1042 | 65.761 | 46.048 | −5.958 | 1.00 | 0.00 | O |
| ATOM | 8166 | N | HIS | A1043 | 68.768 | 48.223 | −8.565 | 1.00 | 0.00 | N |
| ATOM | 8167 | CA | HIS | A1043 | 69.947 | 48.416 | −9.402 | 1.00 | 0.00 | C |
| ATOM | 8168 | C | HIS | A1043 | 71.217 | 48.516 | −8.570 | 1.00 | 0.00 | C |
| ATOM | 8169 | O | HIS | A1043 | 71.268 | 49.239 | −7.577 | 1.00 | 0.00 | O |
| ATOM | 8170 | CB | HIS | A1043 | 69.751 | 49.677 | −10.242 | 1.00 | 0.00 | C |
| ATOM | 8171 | CG | HIS | A1043 | 68.444 | 49.700 | −10.971 | 1.00 | 0.00 | C |
| ATOM | 8172 | ND1 | HIS | A1043 | 68.287 | 49.175 | −12.236 | 1.00 | 0.00 | N |
| ATOM | 8173 | CD2 | HIS | A1043 | 67.212 | 50.095 | −10.574 | 1.00 | 0.00 | C |
| ATOM | 8174 | CE1 | HIS | A1043 | 67.015 | 49.242 | −12.585 | 1.00 | 0.00 | C |
| ATOM | 8175 | NE2 | HIS | A1043 | 66.340 | 49.795 | −11.593 | 1.00 | 0.00 | N |
| ATOM | 8176 | N | SER | A1044 | 72.242 | 47.788 | −8.995 | 1.00 | 0.00 | N |
| ATOM | 8177 | CA | SER | A1044 | 73.511 | 47.763 | −8.283 | 1.00 | 0.00 | C |
| ATOM | 8178 | C | SER | A1044 | 74.440 | 48.914 | −8.643 | 1.00 | 0.00 | C |
| ATOM | 8179 | O | SER | A1044 | 74.055 | 49.766 | −9.475 | 1.00 | 0.00 | O |
| ATOM | 8180 | CB | SER | A1044 | 74.215 | 46.425 | −8.533 | 1.00 | 0.00 | C |
| ATOM | 8181 | OG | SER | A1044 | 74.201 | 46.089 | −9.912 | 1.00 | 0.00 | O |
| ATOM | 8182 | OXT | SER | A1044 | 75.551 | 48.943 | −8.073 | 1.00 | 0.00 | O |
| ATOM | 8183 | OH2 | WAT | W 1 | 41.976 | 63.654 | −7.154 | 1.00 | 0.00 | O |
| ATOM | 8184 | OH2 | WAT | W 2 | 53.602 | 65.013 | −19.781 | 1.00 | 0.00 | O |
| ATOM | 8185 | OH2 | WAT | W 3 | 39.163 | 63.047 | −19.191 | 1.00 | 0.00 | O |
| ATOM | 8186 | OH2 | WAT | W 4 | 52.126 | 54.294 | −4.852 | 1.00 | 0.00 | O |
| ATOM | 8187 | OH2 | WAT | W 5 | 56.134 | 53.565 | −0.644 | 1.00 | 0.00 | O |
| ATOM | 8188 | OH2 | WAT | W 6 | 31.389 | 50.074 | −24.074 | 1.00 | 0.00 | O |
| ATOM | 8189 | OH2 | WAT | W 7 | 49.834 | 48.640 | 1.272 | 1.00 | 0.00 | O |
| ATOM | 8190 | OH2 | WAT | W 8 | 36.988 | 57.887 | 13.369 | 1.00 | 0.00 | O |
| ATOM | 8191 | OH2 | WAT | W 9 | 26.754 | 69.185 | −9.245 | 1.00 | 0.00 | O |
| ATOM | 8192 | OH2 | WAT | W 10 | 39.317 | 64.867 | −14.914 | 1.00 | 0.00 | O |
| ATOM | 8193 | OH2 | WAT | W 11 | 34.207 | 58.774 | −8.708 | 1.00 | 0.00 | O |
| ATOM | 8194 | OH2 | WAT | W 12 | 60.950 | 59.834 | −8.299 | 1.00 | 0.00 | O |
| ATOM | 8195 | OH2 | WAT | W 13 | 36.632 | 72.830 | 0.193 | 1.00 | 0.00 | O |
| ATOM | 8196 | OH2 | WAT | W 14 | 31.706 | 47.180 | −8.087 | 1.00 | 0.00 | O |
| ATOM | 8197 | OH2 | WAT | W 15 | 30.145 | 55.933 | 17.234 | 1.00 | 0.00 | O |
| ATOM | 8198 | OH2 | WAT | W 16 | 26.118 | 49.838 | −13.748 | 1.00 | 0.00 | O |
| ATOM | 8199 | OH2 | WAT | W 17 | 37.626 | 52.387 | −21.143 | 1.00 | 0.00 | O |
| ATOM | 8200 | OH2 | WAT | W 18 | 33.009 | 62.953 | 0.156 | 1.00 | 0.00 | O |
| ATOM | 8201 | OH2 | WAT | W 19 | 24.690 | 53.036 | −11.756 | 1.00 | 0.00 | O |
| ATOM | 8202 | OH2 | WAT | W 20 | 63.104 | 61.520 | −7.574 | 1.00 | 0.00 | O |
| ATOM | 8203 | OH2 | WAT | W 21 | 41.281 | 59.157 | 13.525 | 1.00 | 0.00 | O |
| ATOM | 8204 | OH2 | WAT | W 22 | 47.275 | 55.960 | −15.370 | 1.00 | 0.00 | O |
| ATOM | 8205 | OH2 | WAT | W 23 | 56.384 | 55.894 | −2.113 | 1.00 | 0.00 | O |
| ATOM | 8206 | OH2 | WAT | W 24 | 67.346 | 60.849 | −5.662 | 1.00 | 0.00 | O |
| ATOM | 8207 | OH2 | WAT | W 25 | 26.262 | 48.932 | −10.899 | 1.00 | 0.00 | O |
| ATOM | 8208 | OH2 | WAT | W 26 | 65.624 | 60.393 | −7.594 | 1.00 | 0.00 | O |
| ATOM | 8209 | OH2 | WAT | W 27 | 32.468 | 60.313 | −1.882 | 1.00 | 0.00 | O |
| ATOM | 8210 | OH2 | WAT | W 28 | 20.134 | 54.845 | 16.209 | 1.00 | 0.00 | O |
| ATOM | 8211 | OH2 | WAT | W 29 | 23.817 | 55.676 | −23.389 | 1.00 | 0.00 | O |
| ATOM | 8212 | OH2 | WAT | W 30 | 39.332 | 57.560 | 14.798 | 1.00 | 0.00 | O |
| ATOM | 8213 | OH2 | WAT | W 31 | 20.347 | 58.955 | −21.877 | 1.00 | 0.00 | O |
| ATOM | 8214 | OH2 | WAT | W 32 | 28.078 | 61.165 | 19.428 | 1.00 | 0.00 | O |
| ATOM | 8215 | OH2 | WAT | W 33 | 34.054 | 56.485 | −26.253 | 1.00 | 0.00 | O |
| ATOM | 8216 | OH2 | WAT | W 34 | 26.331 | 40.115 | 7.990 | 1.00 | 0.00 | O |
| ATOM | 8217 | OH2 | WAT | W 35 | 63.797 | 50.314 | −14.009 | 1.00 | 0.00 | O |
| ATOM | 8218 | OH2 | WAT | W 36 | 37.488 | 57.200 | 1.740 | 1.00 | 0.00 | O |
| ATOM | 8219 | OH2 | WAT | W 37 | 24.086 | 42.128 | 6.854 | 1.00 | 0.00 | O |
| ATOM | 8220 | OH2 | WAT | W 38 | 31.954 | 65.565 | 18.957 | 1.00 | 0.00 | O |
| ATOM | 8221 | OH2 | WAT | W 39 | 51.497 | 56.794 | −5.609 | 1.00 | 0.00 | O |
| ATOM | 8222 | OH2 | WAT | W 40 | 20.046 | 56.194 | 7.116 | 1.00 | 0.00 | O |
| ATOM | 8223 | OH2 | WAT | W 41 | 28.269 | 43.982 | 13.133 | 1.00 | 0.00 | O |
| ATOM | 8224 | OH2 | WAT | W 42 | 30.246 | 58.625 | −11.781 | 1.00 | 0.00 | O |
| ATOM | 8225 | OH2 | WAT | W 43 | 64.887 | 59.403 | −3.625 | 1.00 | 0.00 | O |
| ATOM | 8226 | OH2 | WAT | W 44 | 46.354 | 67.851 | −15.580 | 1.00 | 0.00 | O |
| ATOM | 8227 | OH2 | WAT | W 45 | 60.700 | 58.458 | −2.886 | 1.00 | 0.00 | O |
| ATOM | 8228 | OH2 | WAT | W 46 | 60.504 | 62.266 | −1.281 | 1.00 | 0.00 | O |
| ATOM | 8229 | OH2 | WAT | W 47 | 53.603 | 60.069 | −9.457 | 1.00 | 0.00 | O |
| ATOM | 8230 | OH2 | WAT | W 48 | 18.568 | 51.200 | −12.453 | 1.00 | 0.00 | O |
| ATOM | 8231 | OH2 | WAT | W 49 | 28.872 | 42.920 | −11.945 | 1.00 | 0.00 | O |
| ATOM | 8232 | OH2 | WAT | W 50 | 34.483 | 79.286 | −9.190 | 1.00 | 0.00 | O |
| ATOM | 8233 | OH2 | WAT | W 51 | 21.525 | 58.047 | −7.813 | 1.00 | 0.00 | O |
| ATOM | 8234 | OH2 | WAT | W 52 | 36.981 | 72.016 | −4.631 | 1.00 | 0.00 | O |
| ATOM | 8235 | OH2 | WAT | W 53 | 55.632 | 59.594 | −11.514 | 1.00 | 0.00 | O |
| ATOM | 8236 | OH2 | WAT | W 54 | 36.682 | 44.997 | −5.525 | 1.00 | 0.00 | O |
| ATOM | 8237 | OH2 | WAT | W 55 | 51.278 | 53.462 | −24.046 | 1.00 | 0.00 | O |
| ATOM | 8238 | OH2 | WAT | W 56 | 35.166 | 50.774 | −17.551 | 1.00 | 0.00 | O |
| ATOM | 8239 | OH2 | WAT | W 57 | 60.494 | 54.959 | −0.396 | 1.00 | 0.00 | O |
| ATOM | 8240 | OH2 | WAT | W 58 | 42.906 | 56.985 | 6.186 | 1.00 | 0.00 | O |
| ATOM | 8241 | OH2 | WAT | W 59 | 47.856 | 59.460 | −25.676 | 1.00 | 0.00 | O |
| ATOM | 8242 | OH2 | WAT | W 60 | 37.848 | 73.649 | −2.352 | 1.00 | 0.00 | O |
| ATOM | 8243 | OH2 | WAT | W 61 | 19.399 | 55.466 | 11.527 | 1.00 | 0.00 | O |
| ATOM | 8244 | OH2 | WAT | W 62 | 25.912 | 39.804 | 5.188 | 1.00 | 0.00 | O |
| ATOM | 8245 | OH2 | WAT | W 63 | 23.250 | 54.233 | −3.650 | 1.00 | 0.00 | O |
| ATOM | 8246 | OH2 | WAT | W 64 | 34.293 | 52.607 | −1.080 | 1.00 | 0.00 | O |
| ATOM | 8247 | OH2 | WAT | W 65 | 50.998 | 44.378 | −7.959 | 1.00 | 0.00 | O |
| ATOM | 8248 | OH2 | WAT | W 66 | 37.880 | 59.168 | 11.059 | 1.00 | 0.00 | O |
| ATOM | 8249 | OH2 | WAT | W 67 | 16.629 | 49.159 | 24.044 | 1.00 | 0.00 | O |
| ATOM | 8250 | OH2 | WAT | W 68 | 19.390 | 53.620 | 8.177 | 1.00 | 0.00 | O |
| ATOM | 8251 | OH2 | WAT | W 69 | 11.772 | 51.726 | 14.383 | 1.00 | 0.00 | O |
| ATOM | 8252 | OH2 | WAT | W 70 | 17.140 | 56.375 | 10.222 | 1.00 | 0.00 | O |
| ATOM | 8253 | OH2 | WAT | W 71 | 67.975 | 65.714 | −3.480 | 1.00 | 0.00 | O |
| ATOM | 8254 | OH2 | WAT | W 72 | 22.819 | 53.270 | −24.432 | 1.00 | 0.00 | O |
| ATOM | 8255 | OH2 | WAT | W 73 | 52.054 | 50.765 | 6.351 | 1.00 | 0.00 | O |
| ATOM | 8256 | OH2 | WAT | W 74 | 17.599 | 52.928 | 6.337 | 1.00 | 0.00 | O |
| ATOM | 8257 | OH2 | WAT | W 75 | 68.798 | 58.391 | −5.079 | 1.00 | 0.00 | O |
| ATOM | 8258 | OH2 | WAT | W 76 | 33.507 | 48.229 | −10.151 | 1.00 | 0.00 | O |
| ATOM | 8259 | OH2 | WAT | W 77 | 26.790 | 41.641 | −13.180 | 1.00 | 0.00 | O |
| ATOM | 8260 | OH2 | WAT | W 78 | 26.742 | 61.083 | −3.287 | 1.00 | 0.00 | O |
| ATOM | 8261 | OH2 | WAT | W 79 | 49.479 | 62.451 | −8.645 | 1.00 | 0.00 | O |
| ATOM | 8262 | OH2 | WAT | W 80 | 41.025 | 52.500 | −17.001 | 1.00 | 0.00 | O |
| ATOM | 8263 | OH2 | WAT | W 81 | 31.432 | 76.939 | −6.261 | 1.00 | 0.00 | O |
| ATOM | 8264 | OH2 | WAT | W 82 | 43.372 | 68.424 | −24.901 | 1.00 | 0.00 | O |
| ATOM | 8265 | OH2 | WAT | W 83 | 32.759 | 81.310 | −11.722 | 1.00 | 0.00 | O |
| ATOM | 8266 | OH2 | WAT | W 84 | 20.528 | 66.418 | −19.640 | 1.00 | 0.00 | O |
| ATOM | 8267 | OH2 | WAT | W 85 | 40.399 | 62.932 | 6.179 | 1.00 | 0.00 | O |
| ATOM | 8268 | OH2 | WAT | W 86 | 42.121 | 47.451 | −24.562 | 1.00 | 0.00 | O |
| ATOM | 8269 | OH2 | WAT | W 87 | 44.531 | 42.000 | −0.591 | 1.00 | 0.00 | O |
| ATOM | 8270 | OH2 | WAT | W 88 | 38.621 | 55.297 | −31.696 | 1.00 | 0.00 | O |
| ATOM | 8271 | OH2 | WAT | W 89 | 22.298 | 54.497 | −11.948 | 1.00 | 0.00 | O |
| ATOM | 8272 | OH2 | WAT | W 90 | 48.309 | 89.467 | −30.626 | 1.00 | 0.00 | O |
| ATOM | 8273 | OH2 | WAT | W 91 | 36.699 | 51.822 | 3.762 | 1.00 | 0.00 | O |
| ATOM | 8274 | OH2 | WAT | W 92 | 38.737 | 62.445 | 8.410 | 1.00 | 0.00 | O |
| ATOM | 8275 | OH2 | WAT | W 93 | 47.441 | 46.140 | 3.627 | 1.00 | 0.00 | O |
| ATOM | 8276 | OH2 | WAT | W 94 | 33.595 | 57.288 | −35.878 | 1.00 | 0.00 | O |
| ATOM | 8277 | OH2 | WAT | W 95 | 32.313 | 77.764 | −29.564 | 1.00 | 0.00 | O |
| ATOM | 8278 | OH2 | WAT | W 96 | 39.241 | 39.239 | 17.199 | 1.00 | 0.00 | O |
| ATOM | 8279 | OH2 | WAT | W 97 | 35.924 | 53.322 | 1.328 | 1.00 | 0.00 | O |
| ATOM | 8280 | OH2 | WAT | W 98 | 14.453 | 60.152 | 5.057 | 1.00 | 0.00 | O |
| ATOM | 8281 | OH2 | WAT | W 99 | 21.656 | 57.289 | 5.019 | 1.00 | 0.00 | O |
| ATOM | 8282 | OH2 | WAT | W 100 | 43.041 | 48.243 | 11.159 | 1.00 | 0.00 | O |
| ATOM | 8283 | OH2 | WAT | W 101 | 26.081 | 73.002 | 5.562 | 1.00 | 0.00 | O |
| ATOM | 8284 | OH2 | WAT | W 102 | 27.438 | 48.664 | −21.564 | 1.00 | 0.00 | O |
| ATOM | 8285 | OH2 | WAT | W 103 | 33.310 | 78.867 | −4.982 | 1.00 | 0.00 | O |
| ATOM | 8286 | OH2 | WAT | W 104 | 49.995 | 59.656 | −11.589 | 1.00 | 0.00 | O |
| ATOM | 8287 | OH2 | WAT | W 105 | 25.585 | 51.377 | −9.520 | 1.00 | 0.00 | O |
| ATOM | 8288 | OH2 | WAT | W 106 | 40.204 | 56.517 | 5.844 | 1.00 | 0.00 | O |
| ATOM | 8289 | OH2 | WAT | W 107 | 14.014 | 59.888 | −3.434 | 1.00 | 0.00 | O |
| ATOM | 8290 | OH2 | WAT | W 108 | 19.703 | 53.642 | −11.754 | 1.00 | 0.00 | O |
| ATOM | 8291 | OH2 | WAT | W 109 | 26.515 | 63.824 | 17.139 | 1.00 | 0.00 | O |
| ATOM | 8292 | OH2 | WAT | W 110 | 19.778 | 57.566 | −11.797 | 1.00 | 0.00 | O |
| ATOM | 8293 | OH2 | WAT | W 111 | 47.187 | 48.531 | 11.419 | 1.00 | 0.00 | O |
| ATOM | 8294 | OH2 | WAT | W 112 | 67.806 | 81.793 | −25.640 | 1.00 | 0.00 | O |
| ATOM | 8295 | OH2 | WAT | W 113 | 22.910 | 51.819 | −8.744 | 1.00 | 0.00 | O |
| ATOM | 8296 | OH2 | WAT | W 114 | 46.600 | 70.560 | −36.706 | 1.00 | 0.00 | O |
| ATOM | 8297 | OH2 | WAT | W 115 | 20.546 | 57.585 | −5.285 | 1.00 | 0.00 | O |
| ATOM | 8298 | OH2 | WAT | W 116 | 18.164 | 60.620 | 24.435 | 1.00 | 0.00 | O |
| ATOM | 8299 | OH2 | WAT | W 117 | 41.283 | 68.622 | −32.525 | 1.00 | 0.00 | O |
| ATOM | 8300 | OH2 | WAT | W 118 | 38.310 | 40.108 | 1.670 | 1.00 | 0.00 | O |
| ATOM | 8301 | OH2 | WAT | W 119 | 23.864 | 58.245 | 6.322 | 1.00 | 0.00 | O |
| ATOM | 8302 | OH2 | WAT | W 120 | 18.116 | 59.931 | −20.090 | 1.00 | 0.00 | O |
| ATOM | 8303 | OH2 | WAT | W 121 | 41.272 | 77.741 | −14.097 | 1.00 | 0.00 | O |
| ATOM | 8304 | OH2 | WAT | W 122 | 52.834 | 59.194 | −1.694 | 1.00 | 0.00 | O |
| ATOM | 8305 | OH2 | WAT | W 123 | 47.929 | 49.601 | 13.774 | 1.00 | 0.00 | O |
| ATOM | 8306 | OH2 | WAT | W 124 | 35.223 | 43.723 | 28.642 | 1.00 | 0.00 | O |
| ATOM | 8307 | OH2 | WAT | W 125 | 59.493 | 60.742 | −6.299 | 1.00 | 0.00 | O |
| ATOM | 8308 | OH2 | WAT | W 126 | 53.675 | 72.733 | −19.141 | 1.00 | 0.00 | O |
| ATOM | 8309 | OH2 | WAT | W 127 | 39.803 | 54.101 | −20.844 | 1.00 | 0.00 | O |
| ATOM | 8310 | OH2 | WAT | W 128 | 33.123 | 36.777 | −4.396 | 1.00 | 0.00 | O |
| ATOM | 8311 | OH2 | WAT | W 129 | 14.314 | 64.081 | −16.354 | 1.00 | 0.00 | O |
| ATOM | 8312 | OH2 | WAT | W 130 | 47.524 | 43.079 | −2.432 | 1.00 | 0.00 | O |
| ATOM | 8313 | OH2 | WAT | W 131 | 46.908 | 55.986 | −25.481 | 1.00 | 0.00 | O |
| ATOM | 8314 | OH2 | WAT | W 132 | 32.142 | 58.803 | −28.456 | 1.00 | 0.00 | O |
| ATOM | 8315 | OH2 | WAT | W 133 | 49.282 | 50.063 | 28.217 | 1.00 | 0.00 | O |
| ATOM | 8316 | OH2 | WAT | W 134 | 26.302 | 37.406 | 29.348 | 1.00 | 0.00 | O |
| ATOM | 8317 | OH2 | WAT | W 135 | 51.894 | 46.658 | 1.206 | 1.00 | 0.00 | O |
| ATOM | 8318 | OH2 | WAT | W 136 | 41.699 | 58.872 | −16.019 | 1.00 | 0.00 | O |
| ATOM | 8319 | OH2 | WAT | W 137 | 13.825 | 54.259 | 4.434 | 1.00 | 0.00 | O |
| ATOM | 8320 | OH2 | WAT | W 138 | 67.796 | 79.172 | −25.325 | 1.00 | 0.00 | O |
| ATOM | 8321 | OH2 | WAT | W 139 | 42.167 | 75.423 | −16.044 | 1.00 | 0.00 | O |
| ATOM | 8322 | OH2 | WAT | W 140 | 23.124 | 56.190 | 30.804 | 1.00 | 0.00 | O |
| ATOM | 8323 | OH2 | WAT | W 141 | 56.986 | 61.143 | 1.570 | 1.00 | 0.00 | O |
| ATOM | 8324 | OH2 | WAT | W 142 | 20.070 | 74.557 | −6.868 | 1.00 | 0.00 | O |
| ATOM | 8325 | OH2 | WAT | W 143 | 13.368 | 53.676 | 1.802 | 1.00 | 0.00 | O |
| ATOM | 8326 | OH2 | WAT | W 144 | 34.263 | 34.517 | 16.103 | 1.00 | 0.00 | O |
| ATOM | 8327 | OH2 | WAT | W 145 | 33.945 | 64.719 | −32.234 | 1.00 | 0.00 | O |
| ATOM | 8328 | OH2 | WAT | W 146 | 14.059 | 49.309 | 15.412 | 1.00 | 0.00 | O |
| ATOM | 8329 | OH2 | WAT | W 147 | 30.401 | 35.321 | −12.383 | 1.00 | 0.00 | O |
| ATOM | 8330 | OH2 | WAT | W 148 | 18.402 | 55.023 | 14.030 | 1.00 | 0.00 | O |
| ATOM | 8331 | OH2 | WAT | W 149 | 15.633 | 60.472 | 15.485 | 1.00 | 0.00 | O |
| ATOM | 8332 | OH2 | WAT | W 150 | 16.788 | 74.865 | −3.021 | 1.00 | 0.00 | O |
| ATOM | 8333 | OH2 | WAT | W 151 | 56.517 | 46.305 | −17.438 | 1.00 | 0.00 | O |
| ATOM | 8334 | OH2 | WAT | W 152 | 45.631 | 60.108 | −18.238 | 1.00 | 0.00 | O |
| ATOM | 8335 | OH2 | WAT | W 153 | 28.185 | 84.769 | −35.431 | 1.00 | 0.00 | O |
| ATOM | 8336 | OH2 | WAT | W 154 | 73.024 | 65.247 | −7.728 | 1.00 | 0.00 | O |
| ATOM | 8337 | OH2 | WAT | W 155 | 73.780 | 66.204 | −17.660 | 1.00 | 0.00 | O |
| ATOM | 8338 | OH2 | WAT | W 156 | 59.268 | 50.053 | −0.663 | 1.00 | 0.00 | O |
| ATOM | 8339 | OH2 | WAT | W 157 | 23.056 | 78.515 | −24.846 | 1.00 | 0.00 | O |
| ATOM | 8340 | OH2 | WAT | W 158 | 14.298 | 54.490 | −16.078 | 1.00 | 0.00 | O |
| ATOM | 8341 | OH2 | WAT | W 159 | 43.564 | 73.335 | 5.822 | 1.00 | 0.00 | O |
| ATOM | 8342 | OH2 | WAT | W 160 | 11.895 | 55.196 | −12.500 | 1.00 | 0.00 | O |
| ATOM | 8343 | OH2 | WAT | W 161 | 63.546 | 44.759 | −6.927 | 1.00 | 0.00 | O |
| ATOM | 8344 | OH2 | WAT | W 162 | 24.751 | 51.137 | −24.137 | 1.00 | 0.00 | O |
| ATOM | 8345 | OH2 | WAT | W 163 | 35.483 | 45.434 | −27.391 | 1.00 | 0.00 | O |
| ATOM | 8346 | OH2 | WAT | W 164 | 28.707 | 49.870 | −24.430 | 1.00 | 0.00 | O |
| ATOM | 8347 | OH2 | WAT | W 165 | 54.814 | 81.194 | −23.475 | 1.00 | 0.00 | O |
| ATOM | 8348 | OH2 | WAT | W 166 | 23.601 | 63.813 | 7.819 | 1.00 | 0.00 | O |
| ATOM | 8349 | OH2 | WAT | W 167 | 49.745 | 57.750 | −7.642 | 1.00 | 0.00 | O |
| ATOM | 8350 | OH2 | WAT | W 168 | 46.072 | 71.136 | 0.115 | 1.00 | 0.00 | O |
| ATOM | 8351 | OH2 | WAT | W 169 | 24.320 | 55.824 | −1.913 | 1.00 | 0.00 | O |
| ATOM | 8352 | OH2 | WAT | W 170 | 28.642 | 41.362 | −21.793 | 1.00 | 0.00 | O |
| ATOM | 8353 | OH2 | WAT | W 171 | 37.052 | 45.715 | 27.462 | 1.00 | 0.00 | O |
| ATOM | 8354 | OH2 | WAT | W 172 | 41.481 | 41.357 | −17.624 | 1.00 | 0.00 | O |
| ATOM | 8355 | OH2 | WAT | W 173 | 40.307 | 62.729 | −31.958 | 1.00 | 0.00 | O |
| ATOM | 8356 | OH2 | WAT | W 174 | 49.315 | 59.394 | −28.703 | 1.00 | 0.00 | O |
| ATOM | 8357 | OH2 | WAT | W 175 | 64.406 | 39.380 | −10.443 | 1.00 | 0.00 | O |
| ATOM | 8358 | OH2 | WAT | W 176 | 19.161 | 72.628 | 2.904 | 1.00 | 0.00 | O |
| ATOM | 8359 | OH2 | WAT | W 177 | 17.057 | 70.449 | −16.414 | 1.00 | 0.00 | O |
| ATOM | 8360 | OH2 | WAT | W 178 | 9.539 | 52.641 | 16.836 | 1.00 | 0.00 | O |
| ATOM | 8361 | OH2 | WAT | W 179 | 53.885 | 71.468 | −36.741 | 1.00 | 0.00 | O |
| ATOM | 8362 | OH2 | WAT | W 180 | 50.902 | 71.060 | −23.481 | 1.00 | 0.00 | O |
| ATOM | 8363 | OH2 | WAT | W 181 | 49.374 | 44.299 | 4.432 | 1.00 | 0.00 | O |
| ATOM | 8364 | OH2 | WAT | W 182 | 59.359 | 68.451 | 2.377 | 1.00 | 0.00 | O |
| ATOM | 8365 | OH2 | WAT | W 183 | 25.436 | 53.879 | 11.076 | 1.00 | 0.00 | O |
| ATOM | 8366 | OH2 | WAT | W 184 | 69.087 | 61.577 | −2.401 | 1.00 | 0.00 | O |
| ATOM | 8367 | OH2 | WAT | W 185 | 58.340 | 77.598 | −38.439 | 1.00 | 0.00 | O |
| ATOM | 8368 | OH2 | WAT | W 186 | 44.378 | 46.203 | 9.940 | 1.00 | 0.00 | O |
| ATOM | 8369 | OH2 | WAT | W 187 | 46.546 | 57.482 | −18.766 | 1.00 | 0.00 | O |
| ATOM | 8370 | OH2 | WAT | W 188 | 33.489 | 83.732 | −10.371 | 1.00 | 0.00 | O |
| ATOM | 8371 | OH2 | WAT | W 189 | 47.049 | 68.676 | −30.437 | 1.00 | 0.00 | O |
| ATOM | 8372 | OH2 | WAT | W 190 | 44.368 | 56.164 | −15.428 | 1.00 | 0.00 | O |
| ATOM | 8373 | OH2 | WAT | W 191 | 13.141 | 67.038 | −4.766 | 1.00 | 0.00 | O |
| ATOM | 8374 | OH2 | WAT | W 192 | 24.521 | 39.279 | −11.477 | 1.00 | 0.00 | O |
| ATOM | 8375 | OH2 | WAT | W 193 | 41.586 | 61.285 | −29.726 | 1.00 | 0.00 | O |
| ATOM | 8376 | OH2 | WAT | W 194 | 27.184 | 41.484 | 13.312 | 1.00 | 0.00 | O |
| ATOM | 8377 | OH2 | WAT | W 195 | 41.986 | 88.026 | −40.822 | 1.00 | 0.00 | O |
| ATOM | 8378 | OH2 | WAT | W 196 | 69.847 | 70.116 | −12.640 | 1.00 | 0.00 | O |
| ATOM | 8379 | OH2 | WAT | W 197 | 64.131 | 78.076 | −35.187 | 1.00 | 0.00 | O |
| ATOM | 8380 | OH2 | WAT | W 198 | 20.419 | 65.482 | 7.650 | 1.00 | 0.00 | O |
| ATOM | 8381 | OH2 | WAT | W 199 | 11.387 | 61.513 | −18.591 | 1.00 | 0.00 | O |
| ATOM | 8382 | OH2 | WAT | W 200 | 24.508 | 70.807 | −8.820 | 1.00 | 0.00 | O |
| ATOM | 8383 | OH2 | WAT | W 201 | 25.021 | 40.148 | 14.199 | 1.00 | 0.00 | O |
| ATOM | 8384 | OH2 | WAT | W 202 | 22.730 | 63.440 | −33.177 | 1.00 | 0.00 | O |
| ATOM | 8385 | OH2 | WAT | W 203 | 41.675 | 43.444 | 7.942 | 1.00 | 0.00 | O |
| ATOM | 8386 | OH2 | WAT | W 204 | 21.031 | 51.927 | 47.241 | 1.00 | 0.00 | O |
| ATOM | 8387 | OH2 | WAT | W 205 | 41.315 | 50.431 | 33.202 | 1.00 | 0.00 | O |
| ATOM | 8388 | OH2 | WAT | W 206 | 45.964 | 52.942 | −19.262 | 1.00 | 0.00 | O |
| ATOM | 8389 | OH2 | WAT | W 207 | 51.437 | 76.535 | −40.973 | 1.00 | 0.00 | O |
| ATOM | 8390 | OH2 | WAT | W 208 | 25.691 | 86.538 | −31.936 | 1.00 | 0.00 | O |
| ATOM | 8391 | OH2 | WAT | W 209 | 12.475 | 56.456 | 5.405 | 1.00 | 0.00 | O |
| ATOM | 8392 | OH2 | WAT | W 210 | 22.531 | 56.979 | −34.126 | 1.00 | 0.00 | O |
| ATOM | 8393 | OH2 | WAT | W 211 | 43.855 | 72.890 | −42.023 | 1.00 | 0.00 | O |
| ATOM | 8394 | OH2 | WAT | W 212 | 44.587 | 58.897 | −15.991 | 1.00 | 0.00 | O |
| ATOM | 8395 | OH2 | WAT | W 213 | 31.317 | 81.888 | 0.492 | 1.00 | 0.00 | O |
| ATOM | 8396 | OH2 | WAT | W 214 | 51.998 | 56.511 | −30.251 | 1.00 | 0.00 | O |
| ATOM | 8397 | OH2 | WAT | W 215 | 17.284 | 70.213 | −23.398 | 1.00 | 0.00 | O |
| ATOM | 8398 | OH2 | WAT | W 216 | 37.672 | 47.143 | 29.765 | 1.00 | 0.00 | O |
| ATOM | 8399 | OH2 | WAT | W 217 | 49.760 | 55.193 | −28.929 | 1.00 | 0.00 | O |
| ATOM | 8400 | OH2 | WAT | W 218 | 36.216 | 79.446 | −5.014 | 1.00 | 0.00 | O |
| ATOM | 8401 | OH2 | WAT | W 219 | 62.263 | 62.760 | 25.718 | 1.00 | 0.00 | O |
| ATOM | 8402 | OH2 | WAT | W 220 | 11.371 | 53.253 | 6.833 | 1.00 | 0.00 | O |
| ATOM | 8403 | OH2 | WAT | W 221 | 13.906 | 60.932 | −22.496 | 1.00 | 0.00 | O |
| ATOM | 8404 | OH2 | WAT | W 222 | 50.825 | 62.146 | 26.284 | 1.00 | 0.00 | O |
| ATOM | 8405 | OH2 | WAT | W 223 | 68.567 | 79.131 | −18.097 | 1.00 | 0.00 | O |
| ATOM | 8406 | OH2 | WAT | W 224 | 84.222 | 68.534 | −17.389 | 1.00 | 0.00 | O |
| ATOM | 8407 | OH2 | WAT | W 225 | 21.708 | 40.465 | −8.858 | 1.00 | 0.00 | O |
| ATOM | 8408 | OH2 | WAT | W 226 | 52.787 | 69.944 | 18.315 | 1.00 | 0.00 | O |
| ATOM | 8409 | OH2 | WAT | W 227 | 42.689 | 53.369 | 35.556 | 1.00 | 0.00 | O |
| ATOM | 8410 | OH2 | WAT | W 228 | 70.589 | 52.247 | −12.696 | 1.00 | 0.00 | O |
| ATOM | 8411 | OH2 | WAT | W 229 | 34.684 | 70.515 | 13.167 | 1.00 | 0.00 | O |
| ATOM | 8412 | OH2 | WAT | W 230 | 27.167 | 46.423 | 13.589 | 1.00 | 0.00 | O |
| ATOM | 8413 | OH2 | WAT | W 231 | 14.456 | 49.790 | 19.863 | 1.00 | 0.00 | O |
| ATOM | 8414 | OH2 | WAT | W 232 | 49.685 | 73.595 | −21.805 | 1.00 | 0.00 | O |
| ATOM | 8415 | OH2 | WAT | W 233 | 48.689 | 44.406 | −6.194 | 1.00 | 0.00 | O |
| ATOM | 8416 | OH2 | WAT | W 234 | 61.410 | 77.073 | −39.603 | 1.00 | 0.00 | O |
| ATOM | 8417 | OH2 | WAT | W 235 | 22.884 | 78.730 | −30.339 | 1.00 | 0.00 | O |
| ATOM | 8418 | OH2 | WAT | W 236 | 43.601 | 88.814 | −38.504 | 1.00 | 0.00 | O |
| ATOM | 8419 | OH2 | WAT | W 237 | 35.756 | 77.733 | −7.320 | 1.00 | 0.00 | O |
| ATOM | 8420 | OH2 | WAT | W 238 | 24.910 | 68.451 | 21.795 | 1.00 | 0.00 | O |
| ATOM | 8421 | OH2 | WAT | W 239 | 68.065 | 49.290 | −1.106 | 1.00 | 0.00 | O |
| ATOM | 8422 | OH2 | WAT | W 240 | 54.306 | 48.006 | 15.594 | 1.00 | 0.00 | O |
| ATOM | 8423 | OH2 | WAT | W 241 | 55.035 | 50.552 | 16.701 | 1.00 | 0.00 | O |
| ATOM | 8424 | OH2 | WAT | W 242 | 39.346 | 48.853 | −32.773 | 1.00 | 0.00 | O |
| ATOM | 8425 | OH2 | WAT | W 243 | 22.715 | 45.375 | −13.470 | 1.00 | 0.00 | O |
| ATOM | 8426 | OH2 | WAT | W 244 | 67.041 | 55.438 | 0.923 | 1.00 | 0.00 | O |
| ATOM | 8427 | OH2 | WAT | W 245 | 47.938 | 44.041 | −8.649 | 1.00 | 0.00 | O |
| ATOM | 8428 | OH2 | WAT | W 246 | 26.916 | 87.947 | −23.675 | 1.00 | 0.00 | O |
| ATOM | 8429 | OH2 | WAT | W 247 | 18.920 | 48.654 | 37.727 | 1.00 | 0.00 | O |
| ATOM | 8430 | OH2 | WAT | W 248 | 83.609 | 67.735 | −22.899 | 1.00 | 0.00 | O |
| ATOM | 8431 | OH2 | WAT | W 249 | 43.071 | 59.942 | −19.005 | 1.00 | 0.00 | O |
| ATOM | 8432 | OH2 | WAT | W 250 | 47.458 | 79.272 | −40.359 | 1.00 | 0.00 | O |
| ATOM | 8433 | OH2 | WAT | W 251 | 8.628 | 57.772 | −6.846 | 1.00 | 0.00 | O |
| ATOM | 8434 | OH2 | WAT | W 252 | 54.854 | 90.442 | −23.490 | 1.00 | 0.00 | O |
| ATOM | 8435 | OH2 | WAT | W 253 | 27.822 | 36.723 | −19.686 | 1.00 | 0.00 | O |
| ATOM | 8436 | OH2 | WAT | W 254 | 72.038 | 58.835 | −11.191 | 1.00 | 0.00 | O |
| ATOM | 8437 | OH2 | WAT | W 255 | 34.312 | 67.938 | 12.419 | 1.00 | 0.00 | O |
| ATOM | 8438 | OH2 | WAT | W 256 | 11.701 | 59.520 | −4.996 | 1.00 | 0.00 | O |
| ATOM | 8439 | OH2 | WAT | W 257 | 23.555 | 46.185 | −15.867 | 1.00 | 0.00 | O |
| ATOM | 8440 | OH2 | WAT | W 258 | 50.165 | 70.949 | −17.614 | 1.00 | 0.00 | O |
| ATOM | 8441 | OH2 | WAT | W 259 | 45.014 | 90.349 | −25.370 | 1.00 | 0.00 | O |
| ATOM | 8442 | OH2 | WAT | W 260 | 16.031 | 72.424 | −19.154 | 1.00 | 0.00 | O |
| ATOM | 8443 | OH2 | WAT | W 261 | 20.063 | 52.665 | −24.366 | 1.00 | 0.00 | O |
| ATOM | 8444 | OH2 | WAT | W 262 | 59.526 | 76.634 | −8.922 | 1.00 | 0.00 | O |
| ATOM | 8445 | OH2 | WAT | W 263 | 66.985 | 60.106 | −27.206 | 1.00 | 0.00 | O |
| ATOM | 8446 | OH2 | WAT | W 264 | 38.574 | 51.579 | −29.892 | 1.00 | 0.00 | O |
| ATOM | 8447 | OH2 | WAT | W 265 | 40.352 | 83.002 | −10.718 | 1.00 | 0.00 | O |
| ATOM | 8448 | OH2 | WAT | W 266 | 42.228 | 48.810 | 31.272 | 1.00 | 0.00 | O |
| ATOM | 8449 | OH2 | WAT | W 267 | 24.087 | 53.097 | −6.112 | 1.00 | 0.00 | O |
| ATOM | 8450 | OH2 | WAT | W 268 | 53.151 | 47.236 | −24.936 | 1.00 | 0.00 | O |
| ATOM | 8451 | OH2 | WAT | W 269 | 32.606 | 89.327 | −43.042 | 1.00 | 0.00 | O |
| ATOM | 8452 | OH2 | WAT | W 270 | 35.917 | 36.134 | −4.644 | 1.00 | 0.00 | O |
| ATOM | 8453 | OH2 | WAT | W 271 | 70.120 | 83.240 | −29.810 | 1.00 | 0.00 | O |
| ATOM | 8454 | OH2 | WAT | W 272 | 49.674 | 82.612 | −43.733 | 1.00 | 0.00 | O |
| ATOM | 8455 | OH2 | WAT | W 273 | 68.294 | 79.794 | −34.772 | 1.00 | 0.00 | O |
| ATOM | 8456 | OH2 | WAT | W 274 | 29.285 | 46.894 | 37.041 | 1.00 | 0.00 | O |
| ATOM | 8457 | OH2 | WAT | W 275 | 18.501 | 70.060 | 4.192 | 1.00 | 0.00 | O |
| ATOM | 8458 | OH2 | WAT | W 276 | 14.130 | 68.377 | −7.929 | 1.00 | 0.00 | O |
| ATOM | 8459 | OH2 | WAT | W 277 | 50.292 | 72.625 | 9.115 | 1.00 | 0.00 | O |
| ATOM | 8460 | OH2 | WAT | W 278 | 39.681 | 75.872 | −40.189 | 1.00 | 0.00 | O |
| ATOM | 8461 | OH2 | WAT | W 279 | 28.800 | 65.267 | 19.692 | 1.00 | 0.00 | O |
| ATOM | 8462 | OH2 | WAT | W 280 | 11.698 | 58.801 | 7.877 | 1.00 | 0.00 | O |
| ATOM | 8463 | OH2 | WAT | W 281 | 52.681 | 79.071 | −17.318 | 1.00 | 0.00 | O |
| ATOM | 8464 | OH2 | WAT | W 282 | 18.546 | 65.169 | −27.072 | 1.00 | 0.00 | O |
| ATOM | 8465 | OH2 | WAT | W 283 | 44.658 | 79.503 | −33.162 | 1.00 | 0.00 | O |
| ATOM | 8466 | OH2 | WAT | W 284 | 36.159 | 81.248 | 6.301 | 1.00 | 0.00 | O |
| ATOM | 8467 | OH2 | WAT | W 285 | 16.711 | 60.781 | −28.274 | 1.00 | 0.00 | O |
| ATOM | 8468 | OH2 | WAT | W 286 | 29.549 | 87.473 | −17.991 | 1.00 | 0.00 | O |
| ATOM | 8469 | OH2 | WAT | W 287 | 8.872 | 55.103 | −6.123 | 1.00 | 0.00 | O |
| ATOM | 8470 | OH2 | WAT | W 288 | 46.662 | 61.781 | −25.948 | 1.00 | 0.00 | O |
| ATOM | 8471 | OH2 | WAT | W 289 | 42.935 | 82.350 | −10.022 | 1.00 | 0.00 | O |
| ATOM | 8472 | OH2 | WAT | W 290 | 10.298 | 50.821 | 18.871 | 1.00 | 0.00 | O |
| ATOM | 8473 | OH2 | WAT | W 291 | 12.804 | 42.604 | 12.655 | 1.00 | 0.00 | O |
| ATOM | 8474 | OH2 | WAT | W 292 | 35.970 | 45.285 | 30.862 | 1.00 | 0.00 | O |
| ATOM | 8475 | OH2 | WAT | W 293 | 15.720 | 75.427 | −11.770 | 1.00 | 0.00 | O |
| ATOM | 8476 | OH2 | WAT | W 294 | 25.251 | 46.933 | −20.830 | 1.00 | 0.00 | O |
| ATOM | 8477 | OH2 | WAT | W 295 | 56.981 | 65.103 | 24.147 | 1.00 | 0.00 | O |
| ATOM | 8478 | OH2 | WAT | W 296 | 38.675 | 94.386 | −42.056 | 1.00 | 0.00 | O |
| ATOM | 8479 | OH2 | WAT | W 297 | 34.701 | 59.742 | −34.921 | 1.00 | 0.00 | O |
| ATOM | 8480 | OH2 | WAT | W 298 | 32.942 | 41.340 | −17.487 | 1.00 | 0.00 | O |
| ATOM | 8481 | OH2 | WAT | W 299 | 20.659 | 42.773 | −7.510 | 1.00 | 0.00 | O |
| ATOM | 8482 | OH2 | WAT | W 300 | 19.164 | 76.984 | 4.901 | 1.00 | 0.00 | O |
| ATOM | 8483 | OH2 | WAT | W 301 | 43.374 | 80.845 | 4.959 | 1.00 | 0.00 | O |
| ATOM | 8484 | OH2 | WAT | W 302 | 32.892 | 85.489 | −5.433 | 1.00 | 0.00 | O |
| ATOM | 8485 | OH2 | WAT | W 303 | 31.125 | 33.618 | 2.743 | 1.00 | 0.00 | O |
| ATOM | 8486 | OH2 | WAT | W 304 | 21.401 | 78.735 | −1.300 | 1.00 | 0.00 | O |
| ATOM | 8487 | OH2 | WAT | W 305 | 55.248 | 59.205 | 4.018 | 1.00 | 0.00 | O |
| ATOM | 8488 | OH2 | WAT | W 306 | 13.472 | 62.549 | −19.950 | 1.00 | 0.00 | O |
| ATOM | 8489 | OH2 | WAT | W 307 | 16.942 | 51.046 | −19.243 | 1.00 | 0.00 | O |
| ATOM | 8490 | OH2 | WAT | W 308 | 14.306 | 63.006 | −12.128 | 1.00 | 0.00 | O |
| ATOM | 8491 | OH2 | WAT | W 309 | 53.329 | 87.016 | −21.261 | 1.00 | 0.00 | O |
| ATOM | 8492 | OH2 | WAT | W 310 | 70.847 | 49.607 | −0.120 | 1.00 | 0.00 | O |
| ATOM | 8493 | OH2 | WAT | W 311 | 57.958 | 42.552 | 1.480 | 1.00 | 0.00 | O |
| ATOM | 8494 | OH2 | WAT | W 312 | 74.685 | 76.841 | −14.216 | 1.00 | 0.00 | O |
| ATOM | 8495 | OH2 | WAT | W 313 | 24.865 | 83.255 | −16.893 | 1.00 | 0.00 | O |
| ATOM | 8496 | OH2 | WAT | W 314 | 57.004 | 67.948 | −35.397 | 1.00 | 0.00 | O |
| ATOM | 8497 | OH2 | WAT | W 315 | 48.910 | 50.855 | −29.603 | 1.00 | 0.00 | O |
| ATOM | 8498 | OH2 | WAT | W 316 | 66.516 | 71.805 | −6.758 | 1.00 | 0.00 | O |
| ATOM | 8499 | OH2 | WAT | W 317 | 28.750 | 93.957 | −38.397 | 1.00 | 0.00 | O |
| ATOM | 8500 | OH2 | WAT | W 318 | 32.641 | 64.841 | −10.056 | 1.00 | 0.00 | O |
| ATOM | 8501 | OH2 | WAT | W 319 | 13.390 | 51.126 | −1.727 | 1.00 | 0.00 | O |
| ATOM | 8502 | OH2 | WAT | W 320 | 39.218 | 49.401 | 30.125 | 1.00 | 0.00 | O |
| ATOM | 8503 | OH2 | WAT | W 321 | 67.809 | 58.814 | −23.999 | 1.00 | 0.00 | O |
| ATOM | 8504 | OH2 | WAT | W 322 | 20.711 | 59.173 | −34.513 | 1.00 | 0.00 | O |
| ATOM | 8505 | OH2 | WAT | W 323 | 37.626 | 75.871 | −42.367 | 1.00 | 0.00 | O |
| ATOM | 8506 | OH2 | WAT | W 324 | 31.743 | 51.398 | −35.443 | 1.00 | 0.00 | O |
| ATOM | 8507 | OH2 | WAT | W 325 | 12.990 | 71.425 | −0.518 | 1.00 | 0.00 | O |
| ATOM | 8508 | OH2 | WAT | W 326 | 36.948 | 41.131 | −24.831 | 1.00 | 0.00 | O |
| ATOM | 8509 | OH2 | WAT | W 327 | 19.231 | 42.667 | −2.519 | 1.00 | 0.00 | O |
| ATOM | 8510 | OH2 | WAT | W 328 | 49.940 | 44.205 | 7.115 | 1.00 | 0.00 | O |
| ATOM | 8511 | OH2 | WAT | W 329 | 37.339 | 45.566 | −25.533 | 1.00 | 0.00 | O |
| ATOM | 8512 | OH2 | WAT | W 330 | 75.324 | 74.286 | −19.611 | 1.00 | 0.00 | O |
| ATOM | 8513 | OH2 | WAT | W 331 | 50.283 | 61.339 | −31.172 | 1.00 | 0.00 | O |
| ATOM | 8514 | OH2 | WAT | W 332 | 42.968 | 56.925 | −31.533 | 1.00 | 0.00 | O |
| ATOM | 8515 | OH2 | WAT | W 333 | 13.231 | 68.986 | 0.966 | 1.00 | 0.00 | O |
| ATOM | 8516 | OH2 | WAT | W 334 | 40.964 | 83.065 | −31.335 | 1.00 | 0.00 | O |
| ATOM | 8517 | OH2 | WAT | W 335 | 26.935 | 52.485 | 22.429 | 1.00 | 0.00 | O |
| ATOM | 8518 | OH2 | WAT | W 336 | 12.535 | 66.558 | −9.411 | 1.00 | 0.00 | O |
| ATOM | 8519 | OH2 | WAT | W 337 | 42.574 | 39.474 | −15.819 | 1.00 | 0.00 | O |
| ATOM | 8520 | OH2 | WAT | W 338 | 48.063 | 48.452 | 16.338 | 1.00 | 0.00 | O |
| ATOM | 8521 | OH2 | WAT | W 339 | 11.396 | 46.881 | 17.803 | 1.00 | 0.00 | O |
| ATOM | 8522 | OH2 | WAT | W 340 | 16.456 | 67.949 | −19.966 | 1.00 | 0.00 | O |
| ATOM | 8523 | OH2 | WAT | W 341 | 5.127 | 52.960 | −5.987 | 1.00 | 0.00 | O |
| ATOM | 8524 | OH2 | WAT | W 342 | 56.366 | 91.396 | −25.674 | 1.00 | 0.00 | O |
| ATOM | 8525 | OH2 | WAT | W 343 | 39.144 | 83.533 | −27.276 | 1.00 | 0.00 | O |
| ATOM | 8526 | OH2 | WAT | W 344 | 60.906 | 57.044 | 15.729 | 1.00 | 0.00 | O |
| ATOM | 8527 | OH2 | WAT | W 345 | 13.476 | 51.618 | 21.701 | 1.00 | 0.00 | O |
| ATOM | 8528 | OH2 | WAT | W 346 | 43.475 | 94.139 | −37.674 | 1.00 | 0.00 | O |
| ATOM | 8529 | OH2 | WAT | W 347 | 28.005 | 35.390 | −6.921 | 1.00 | 0.00 | O |
| ATOM | 8530 | OH2 | WAT | W 348 | 80.217 | 64.249 | −21.456 | 1.00 | 0.00 | O |
| ATOM | 8531 | OH2 | WAT | W 349 | 51.582 | 45.241 | 9.734 | 1.00 | 0.00 | O |
| ATOM | 8532 | OH2 | WAT | W 350 | 21.442 | 46.768 | −17.471 | 1.00 | 0.00 | O |
| ATOM | 8533 | OH2 | WAT | W 351 | 47.375 | 71.994 | −20.206 | 1.00 | 0.00 | O |
| ATOM | 8534 | OH2 | WAT | W 352 | 21.529 | 86.223 | −30.867 | 1.00 | 0.00 | O |
| ATOM | 8535 | OH2 | WAT | W 353 | 33.020 | 67.074 | 21.359 | 1.00 | 0.00 | O |
| ATOM | 8536 | OH2 | WAT | W 354 | 14.661 | 62.189 | −14.529 | 1.00 | 0.00 | O |
| ATOM | 8537 | OH2 | WAT | W 355 | 50.340 | 73.832 | −25.135 | 1.00 | 0.00 | O |
| ATOM | 8538 | OH2 | WAT | W 356 | 41.981 | 79.529 | −23.664 | 1.00 | 0.00 | O |
| ATOM | 8539 | OH2 | WAT | W 357 | 39.972 | 45.436 | 25.468 | 1.00 | 0.00 | O |
| ATOM | 8540 | OH2 | WAT | W 358 | 50.969 | 76.614 | 14.421 | 1.00 | 0.00 | O |
| ATOM | 8541 | OH2 | WAT | W 359 | 38.542 | 45.259 | −33.621 | 1.00 | 0.00 | O |
| ATOM | 8542 | OH2 | WAT | W 360 | 61.488 | 54.293 | −28.837 | 1.00 | 0.00 | O |
| ATOM | 8543 | OH2 | WAT | W 361 | 53.752 | 46.861 | 23.465 | 1.00 | 0.00 | O |
| ATOM | 8544 | OH2 | WAT | W 362 | 57.003 | 43.245 | −20.158 | 1.00 | 0.00 | O |
| ATOM | 8545 | OH2 | WAT | W 363 | 67.626 | 54.901 | −18.285 | 1.00 | 0.00 | O |
| ATOM | 8546 | OH2 | WAT | W 364 | 42.418 | 80.223 | −31.622 | 1.00 | 0.00 | O |
| ATOM | 8547 | OH2 | WAT | W 365 | 29.083 | 62.415 | −39.783 | 1.00 | 0.00 | O |
| ATOM | 8548 | OH2 | WAT | W 366 | 26.860 | 61.813 | 10.730 | 1.00 | 0.00 | O |
| ATOM | 8549 | OH2 | WAT | W 367 | 45.805 | 45.098 | 22.818 | 1.00 | 0.00 | O |
| ATOM | 8550 | OH2 | WAT | W 368 | 35.137 | 51.286 | 35.769 | 1.00 | 0.00 | O |
| ATOM | 8551 | OH2 | WAT | W 369 | 57.651 | 62.471 | 7.869 | 1.00 | 0.00 | O |
| ATOM | 8552 | OH2 | WAT | W 370 | 25.333 | 33.637 | 13.177 | 1.00 | 0.00 | O |
| ATOM | 8553 | OH2 | WAT | W 371 | 27.654 | 59.510 | 8.691 | 1.00 | 0.00 | O |
| ATOM | 8554 | OH2 | WAT | W 372 | 42.826 | 94.774 | −30.165 | 1.00 | 0.00 | O |
| ATOM | 8555 | OH2 | WAT | W 373 | 13.853 | 58.151 | −1.424 | 1.00 | 0.00 | O |
| ATOM | 8556 | OH2 | WAT | W 374 | 49.192 | 76.578 | 5.501 | 1.00 | 0.00 | O |
| ATOM | 8557 | OH2 | WAT | W 375 | 27.925 | 67.696 | 28.209 | 1.00 | 0.00 | O |
| ATOM | 8558 | OH2 | WAT | W 376 | 41.952 | 39.320 | 16.716 | 1.00 | 0.00 | O |
| ATOM | 8559 | OH2 | WAT | W 377 | 58.773 | 46.532 | −1.499 | 1.00 | 0.00 | O |
| ATOM | 8560 | OH2 | WAT | W 378 | 73.326 | 76.954 | −18.532 | 1.00 | 0.00 | O |
| ATOM | 8561 | OH2 | WAT | W 379 | 19.656 | 39.469 | 17.560 | 1.00 | 0.00 | O |
| ATOM | 8562 | OH2 | WAT | W 380 | 39.710 | 59.119 | −18.070 | 1.00 | 0.00 | O |
| ATOM | 8563 | OH2 | WAT | W 381 | 28.056 | 47.248 | −31.460 | 1.00 | 0.00 | O |
| ATOM | 8564 | OH2 | WAT | W 382 | 68.576 | 47.066 | −16.559 | 1.00 | 0.00 | O |
| ATOM | 8565 | OH2 | WAT | W 383 | 66.502 | 62.821 | −13.285 | 1.00 | 0.00 | O |
| ATOM | 8566 | OH2 | WAT | W 384 | 26.551 | 75.251 | 2.041 | 1.00 | 0.00 | O |
| ATOM | 8567 | OH2 | WAT | W 385 | 39.989 | 39.143 | 9.181 | 1.00 | 0.00 | O |
| ATOM | 8568 | OH2 | WAT | W 386 | 21.546 | 47.172 | 39.121 | 1.00 | 0.00 | O |
| ATOM | 8569 | OH2 | WAT | W 387 | 42.166 | 75.026 | −41.390 | 1.00 | 0.00 | O |
| ATOM | 8570 | OH2 | WAT | W 388 | 14.668 | 55.649 | 27.168 | 1.00 | 0.00 | O |
| ATOM | 8571 | OH2 | WAT | W 389 | 28.635 | 59.881 | −39.092 | 1.00 | 0.00 | O |
| ATOM | 8572 | OH2 | WAT | W 390 | 39.198 | 43.093 | 22.518 | 1.00 | 0.00 | O |
| ATOM | 8573 | OH2 | WAT | W 391 | 16.373 | 52.883 | −22.472 | 1.00 | 0.00 | O |
| ATOM | 8574 | OH2 | WAT | W 392 | 27.249 | 35.030 | 29.245 | 1.00 | 0.00 | O |
| ATOM | 8575 | OH2 | WAT | W 393 | 17.213 | 80.907 | −7.458 | 1.00 | 0.00 | O |
| ATOM | 8576 | OH2 | WAT | W 394 | 48.192 | 41.940 | 3.626 | 1.00 | 0.00 | O |
| ATOM | 8577 | OH2 | WAT | W 395 | 73.444 | 51.506 | −11.329 | 1.00 | 0.00 | O |
| ATOM | 8578 | OH2 | WAT | W 396 | 58.713 | 53.858 | −22.201 | 1.00 | 0.00 | O |
| ATOM | 8579 | OH2 | WAT | W 397 | 47.585 | 79.681 | −18.583 | 1.00 | 0.00 | O |
| ATOM | 8580 | OH2 | WAT | W 398 | 40.821 | 96.690 | −37.270 | 1.00 | 0.00 | O |
| ATOM | 8581 | OH2 | WAT | W 399 | 61.090 | 63.873 | 15.190 | 1.00 | 0.00 | O |
| ATOM | 8582 | OH2 | WAT | W 400 | 59.266 | 37.758 | −15.399 | 1.00 | 0.00 | O |
| ATOM | 8583 | OH2 | WAT | W 401 | 21.130 | 30.440 | 17.589 | 1.00 | 0.00 | O |
| ATOM | 8584 | OH2 | WAT | W 402 | 46.166 | 97.283 | −43.899 | 1.00 | 0.00 | O |
| ATOM | 8585 | OH2 | WAT | W 403 | 21.601 | 86.388 | −19.084 | 1.00 | 0.00 | O |
| ATOM | 8586 | OH2 | WAT | W 404 | 20.559 | 68.460 | 22.165 | 1.00 | 0.00 | O |
| ATOM | 8587 | OH2 | WAT | W 405 | 45.429 | 70.094 | −18.226 | 1.00 | 0.00 | O |
| ATOM | 8588 | OH2 | WAT | W 406 | 17.460 | 70.834 | 6.628 | 1.00 | 0.00 | O |
| ATOM | 8589 | OH2 | WAT | W 407 | 51.178 | 52.977 | −28.541 | 1.00 | 0.00 | O |
| ATOM | 8590 | OH2 | WAT | W 408 | 19.143 | 83.560 | −25.728 | 1.00 | 0.00 | O |
| ATOM | 8591 | OH2 | WAT | W 409 | 40.017 | 86.342 | −21.820 | 1.00 | 0.00 | O |
| ATOM | 8592 | OH2 | WAT | W 410 | 22.588 | 66.313 | 29.105 | 1.00 | 0.00 | O |
| ATOM | 8593 | OH2 | WAT | W 411 | 24.924 | 59.432 | −36.321 | 1.00 | 0.00 | O |
| ATOM | 8594 | OH2 | WAT | W 412 | 58.534 | 34.944 | −5.958 | 1.00 | 0.00 | O |
| ATOM | 8595 | OH2 | WAT | W 413 | 50.571 | 79.325 | −15.679 | 1.00 | 0.00 | O |
| ATOM | 8596 | OH2 | WAT | W 414 | 17.547 | 68.712 | 24.284 | 1.00 | 0.00 | O |
| ATOM | 8597 | OH2 | WAT | W 415 | 56.497 | 93.003 | −29.406 | 1.00 | 0.00 | O |
| ATOM | 8598 | OH2 | WAT | W 416 | 52.563 | 73.376 | 4.325 | 1.00 | 0.00 | O |
| ATOM | 8599 | OH2 | WAT | W 417 | 21.955 | 36.827 | 0.123 | 1.00 | 0.00 | O |
| ATOM | 8600 | OH2 | WAT | W 418 | 40.057 | 79.947 | −28.024 | 1.00 | 0.00 | O |
| ATOM | 8601 | OH2 | WAT | W 419 | 10.491 | 77.324 | −5.020 | 1.00 | 0.00 | O |
| ATOM | 8602 | OH2 | WAT | W 420 | 26.174 | 64.613 | 8.454 | 1.00 | 0.00 | O |
| ATOM | 8603 | OH2 | WAT | W 421 | 72.887 | 64.089 | −21.074 | 1.00 | 0.00 | O |
| ATOM | 8604 | OH2 | WAT | W 422 | 79.850 | 74.111 | −17.731 | 1.00 | 0.00 | O |
| ATOM | 8605 | OH2 | WAT | W 423 | 66.746 | 74.578 | −4.415 | 1.00 | 0.00 | O |
| ATOM | 8606 | OH2 | WAT | W 424 | 48.121 | 64.471 | −28.375 | 1.00 | 0.00 | O |
| ATOM | 8607 | OH2 | WAT | W 425 | 42.562 | 39.570 | −1.498 | 1.00 | 0.00 | O |
| ATOM | 8608 | OH2 | WAT | W 426 | 63.060 | 67.958 | −31.848 | 1.00 | 0.00 | O |
| ATOM | 8609 | OH2 | WAT | W 427 | 24.133 | 42.119 | −14.621 | 1.00 | 0.00 | O |
| ATOM | 8610 | OH2 | WAT | W 428 | 27.601 | 50.987 | 13.315 | 1.00 | 0.00 | O |
| ATOM | 8611 | OH2 | WAT | W 429 | 38.651 | 83.736 | −39.775 | 1.00 | 0.00 | O |
| ATOM | 8612 | OH2 | WAT | W 430 | 76.639 | 74.331 | −26.800 | 1.00 | 0.00 | O |
| ATOM | 8613 | OH2 | WAT | W 431 | 28.832 | 72.523 | 22.366 | 1.00 | 0.00 | O |
| ATOM | 8614 | OH2 | WAT | W 432 | 20.710 | 55.297 | −35.830 | 1.00 | 0.00 | O |
| ATOM | 8615 | OH2 | WAT | W 433 | 24.127 | 57.300 | 38.838 | 1.00 | 0.00 | O |
| ATOM | 8616 | OH2 | WAT | W 434 | 38.116 | 49.320 | −10.479 | 1.00 | 0.00 | O |
| ATOM | 8617 | OH2 | WAT | W 435 | 23.431 | 82.846 | −40.701 | 1.00 | 0.00 | O |
| ATOM | 8618 | OH2 | WAT | W 436 | 17.153 | 67.203 | −26.037 | 1.00 | 0.00 | O |
| ATOM | 8619 | OH2 | WAT | W 437 | 66.929 | 84.509 | −22.081 | 1.00 | 0.00 | O |
| ATOM | 8620 | OH2 | WAT | W 438 | 52.699 | 65.054 | −28.059 | 1.00 | 0.00 | O |
| ATOM | 8621 | OH2 | WAT | W 439 | 20.671 | 68.507 | 8.683 | 1.00 | 0.00 | O |
| ATOM | 8622 | OH2 | WAT | W 440 | 61.541 | 63.964 | −30.852 | 1.00 | 0.00 | O |
| ATOM | 8623 | OH2 | WAT | W 441 | 9.820 | 60.220 | 6.782 | 1.00 | 0.00 | O |
| ATOM | 8624 | OH2 | WAT | W 442 | 31.578 | 86.047 | −19.897 | 1.00 | 0.00 | O |
| ATOM | 8625 | OH2 | WAT | W 443 | 28.116 | 88.069 | −41.601 | 1.00 | 0.00 | O |
| ATOM | 8626 | OH2 | WAT | W 444 | 44.813 | 82.813 | −30.442 | 1.00 | 0.00 | O |
| ATOM | 8627 | OH2 | WAT | W 445 | 59.411 | 67.316 | −34.553 | 1.00 | 0.00 | O |
| ATOM | 8628 | OH2 | WAT | W 446 | 70.281 | 56.737 | −12.641 | 1.00 | 0.00 | O |
| ATOM | 8629 | OH2 | WAT | W 447 | 46.133 | 46.111 | 16.197 | 1.00 | 0.00 | O |
| ATOM | 8630 | OH2 | WAT | W 448 | 46.022 | 82.991 | −25.291 | 1.00 | 0.00 | O |
| ATOM | 8631 | OH2 | WAT | W 449 | 56.919 | 87.865 | −19.906 | 1.00 | 0.00 | O |
| ATOM | 8632 | OH2 | WAT | W 450 | 73.671 | 74.275 | −5.837 | 1.00 | 0.00 | O |
| ATOM | 8633 | OH2 | WAT | W 451 | 19.790 | 43.342 | 33.818 | 1.00 | 0.00 | O |
| ATOM | 8634 | OH2 | WAT | W 452 | 79.363 | 51.263 | −0.742 | 1.00 | 0.00 | O |
| ATOM | 8635 | OH2 | WAT | W 453 | 65.997 | 45.465 | −3.356 | 1.00 | 0.00 | O |
| ATOM | 8636 | OH2 | WAT | W 454 | 48.283 | 78.082 | −9.662 | 1.00 | 0.00 | O |
| ATOM | 8637 | OH2 | WAT | W 455 | 30.091 | 34.272 | −1.319 | 1.00 | 0.00 | O |
| ATOM | 8638 | OH2 | WAT | W 456 | 16.243 | 75.141 | −24.079 | 1.00 | 0.00 | O |
| ATOM | 8639 | OH2 | WAT | W 457 | 17.563 | 39.586 | 35.890 | 1.00 | 0.00 | O |
| ATOM | 8640 | OH2 | WAT | W 458 | 22.610 | 31.267 | 29.293 | 1.00 | 0.00 | O |
| ATOM | 8641 | OH2 | WAT | W 459 | 33.437 | 84.502 | −18.287 | 1.00 | 0.00 | O |
| ATOM | 8642 | OH2 | WAT | W 460 | 41.281 | 95.077 | −39.430 | 1.00 | 0.00 | O |
| ATOM | 8643 | OH2 | WAT | W 461 | 44.192 | 83.798 | −27.708 | 1.00 | 0.00 | O |
| ATOM | 8644 | OH2 | WAT | W 462 | 30.849 | 57.457 | 4.483 | 1.00 | 0.00 | O |
| ATOM | 8645 | OH2 | WAT | W 463 | 65.863 | 66.367 | −0.404 | 1.00 | 0.00 | O |
| ATOM | 8646 | OH2 | WAT | W 464 | 46.505 | 68.680 | 26.433 | 1.00 | 0.00 | O |
| ATOM | 8647 | OH2 | WAT | W 465 | 70.068 | 61.945 | −20.523 | 1.00 | 0.00 | O |
| ATOM | 8648 | OH2 | WAT | W 466 | 23.627 | 91.160 | −25.962 | 1.00 | 0.00 | O |
| ATOM | 8649 | OH2 | WAT | W 467 | 25.303 | 71.234 | 22.351 | 1.00 | 0.00 | O |
| ATOM | 8650 | OH2 | WAT | W 468 | 13.916 | 69.555 | 26.531 | 1.00 | 0.00 | O |
| ATOM | 8651 | OH2 | WAT | W 469 | 62.187 | 80.969 | −19.167 | 1.00 | 0.00 | O |
| ATOM | 8652 | OH2 | WAT | W 470 | 27.688 | 47.850 | 45.907 | 1.00 | 0.00 | O |
| ATOM | 8653 | OH2 | WAT | W 471 | 24.867 | 34.296 | 0.230 | 1.00 | 0.00 | O |
| ATOM | 8654 | OH2 | WAT | W 472 | 57.552 | 69.077 | 13.347 | 1.00 | 0.00 | O |
| ATOM | 8655 | OH2 | WAT | W 473 | 7.919 | 48.004 | 11.809 | 1.00 | 0.00 | O |
| ATOM | 8656 | OH2 | WAT | W 474 | 71.345 | 59.933 | 0.846 | 1.00 | 0.00 | O |
| ATOM | 8657 | OH2 | WAT | W 475 | 67.625 | 98.259 | −23.332 | 1.00 | 0.00 | O |
| ATOM | 8658 | OH2 | WAT | W 476 | 12.293 | 75.293 | −13.586 | 1.00 | 0.00 | O |
| ATOM | 8659 | OH2 | WAT | W 477 | 28.034 | 52.152 | 42.304 | 1.00 | 0.00 | O |
| ATOM | 8660 | OH2 | WAT | W 478 | 22.101 | 62.991 | 33.579 | 1.00 | 0.00 | O |
| ATOM | 8661 | OH2 | WAT | W 479 | 59.350 | 67.017 | −26.349 | 1.00 | 0.00 | O |
| ATOM | 8662 | OH2 | WAT | W 480 | 34.153 | 87.410 | −10.585 | 1.00 | 0.00 | O |
| ATOM | 8663 | OH2 | WAT | W 481 | 30.242 | 36.569 | 29.051 | 1.00 | 0.00 | O |
| ATOM | 8664 | OH2 | WAT | W 482 | 8.630 | 49.745 | 5.592 | 1.00 | 0.00 | O |
| ATOM | 8665 | OH2 | WAT | W 483 | 41.971 | 67.468 | 29.311 | 1.00 | 0.00 | O |
| ATOM | 8666 | OH2 | WAT | W 484 | 72.488 | 75.946 | −27.178 | 1.00 | 0.00 | O |
| ATOM | 8667 | OH2 | WAT | W 485 | 8.808 | 44.630 | 12.847 | 1.00 | 0.00 | O |
| ATOM | 8668 | OH2 | WAT | W 486 | 71.066 | 43.252 | −15.447 | 1.00 | 0.00 | O |
| ATOM | 8669 | OH2 | WAT | W 487 | 41.610 | 44.823 | −24.420 | 1.00 | 0.00 | O |
| ATOM | 8670 | OH2 | WAT | W 488 | 39.942 | 66.977 | 31.394 | 1.00 | 0.00 | O |
| ATOM | 8671 | OH2 | WAT | W 489 | 53.306 | 52.736 | 26.224 | 1.00 | 0.00 | O |
| ATOM | 8672 | OH2 | WAT | W 490 | 26.040 | 44.611 | −27.019 | 1.00 | 0.00 | O |
| ATOM | 8673 | OH2 | WAT | W 491 | 19.640 | 52.648 | −27.164 | 1.00 | 0.00 | O |
| ATOM | 8674 | OH2 | WAT | W 492 | 71.088 | 95.502 | −34.983 | 1.00 | 0.00 | O |
| ATOM | 8675 | OH2 | WAT | W 493 | 48.006 | 64.627 | −25.755 | 1.00 | 0.00 | O |
| ATOM | 8676 | OH2 | WAT | W 494 | 44.643 | 64.544 | −35.180 | 1.00 | 0.00 | O |
| ATOM | 8677 | OH2 | WAT | W 495 | 17.848 | 53.127 | 35.582 | 1.00 | 0.00 | O |
| ATOM | 8678 | OH2 | WAT | W 496 | 83.559 | 70.263 | −20.332 | 1.00 | 0.00 | O |
| ATOM | 8679 | OH2 | WAT | W 497 | 43.889 | 79.656 | −42.656 | 1.00 | 0.00 | O |
| ATOM | 8680 | OH2 | WAT | W 498 | 22.659 | 62.756 | 16.175 | 1.00 | 0.00 | O |
| ATOM | 8681 | OH2 | WAT | W 499 | 45.784 | 79.442 | −8.085 | 1.00 | 0.00 | O |
| ATOM | 8682 | OH2 | WAT | W 500 | 26.627 | 88.208 | −4.106 | 1.00 | 0.00 | O |
| ATOM | 8683 | OH2 | WAT | W 501 | 38.549 | 42.119 | −22.265 | 1.00 | 0.00 | O |
| ATOM | 8684 | OH2 | WAT | W 502 | 36.969 | 29.333 | 12.945 | 1.00 | 0.00 | O |
| ATOM | 8685 | OH2 | WAT | W 503 | 20.361 | 65.667 | 27.146 | 1.00 | 0.00 | O |
| ATOM | 8686 | OH2 | WAT | W 504 | 6.748 | 59.445 | 2.879 | 1.00 | 0.00 | O |
| ATOM | 8687 | OH2 | WAT | W 505 | 61.176 | 77.604 | −12.987 | 1.00 | 0.00 | O |
| ATOM | 8688 | OH2 | WAT | W 506 | 28.054 | 49.328 | 38.513 | 1.00 | 0.00 | O |
| ATOM | 8689 | OH2 | WAT | W 507 | 29.119 | 41.162 | −25.731 | 1.00 | 0.00 | O |
| ATOM | 8690 | OH2 | WAT | W 508 | 19.438 | 67.582 | −2.809 | 1.00 | 0.00 | O |
| ATOM | 8691 | OH2 | WAT | W 509 | 43.444 | 41.879 | −7.624 | 1.00 | 0.00 | O |
| ATOM | 8692 | OH2 | WAT | W 510 | 48.772 | 55.128 | −22.658 | 1.00 | 0.00 | O |
| ATOM | 8693 | OH2 | WAT | W 511 | 26.320 | 81.968 | −3.668 | 1.00 | 0.00 | O |
| ATOM | 8694 | OH2 | WAT | W 512 | 35.820 | 38.968 | −16.233 | 1.00 | 0.00 | O |
| ATOM | 8695 | OH2 | WAT | W 513 | 58.609 | 51.285 | 22.212 | 1.00 | 0.00 | O |
| ATOM | 8696 | OH2 | WAT | W 514 | 54.001 | 45.745 | 4.789 | 1.00 | 0.00 | O |
| ATOM | 8697 | OH2 | WAT | W 515 | 46.519 | 80.472 | −42.691 | 1.00 | 0.00 | O |
| ATOM | 8698 | OH2 | WAT | W 516 | 71.771 | 84.685 | −19.911 | 1.00 | 0.00 | O |
| ATOM | 8699 | OH2 | WAT | W 517 | 61.087 | 67.591 | −36.581 | 1.00 | 0.00 | O |
| ATOM | 8700 | OH2 | WAT | W 518 | 38.313 | 31.520 | 16.740 | 1.00 | 0.00 | O |
| ATOM | 8701 | OH2 | WAT | W 519 | 58.354 | 34.922 | −12.186 | 1.00 | 0.00 | O |
| ATOM | 8702 | OH2 | WAT | W 520 | 21.065 | 79.730 | −23.655 | 1.00 | 0.00 | O |
| ATOM | 8703 | OH2 | WAT | W 521 | 51.271 | 71.537 | −20.143 | 1.00 | 0.00 | O |
| ATOM | 8704 | OH2 | WAT | W 522 | 32.007 | 85.367 | −8.663 | 1.00 | 0.00 | O |
| ATOM | 8705 | OH2 | WAT | W 523 | 21.559 | 29.241 | 27.796 | 1.00 | 0.00 | O |
| ATOM | 8706 | OH2 | WAT | W 524 | 21.899 | 87.695 | −6.948 | 1.00 | 0.00 | O |
| ATOM | 8707 | OH2 | WAT | W 525 | 31.622 | 57.574 | 37.384 | 1.00 | 0.00 | O |
| ATOM | 8708 | OH2 | WAT | W 526 | 17.396 | 81.625 | −17.128 | 1.00 | 0.00 | O |
| ATOM | 8709 | OH2 | WAT | W 527 | 51.982 | 59.266 | 6.190 | 1.00 | 0.00 | O |
| ATOM | 8710 | OH2 | WAT | W 528 | 22.034 | 80.360 | −35.984 | 1.00 | 0.00 | O |
| ATOM | 8711 | OH2 | WAT | W 529 | 25.516 | 74.205 | 8.628 | 1.00 | 0.00 | O |
| ATOM | 8712 | OH2 | WAT | W 530 | 21.692 | 80.084 | 5.351 | 1.00 | 0.00 | O |
| ATOM | 8713 | OH2 | WAT | W 531 | 48.615 | 94.243 | −37.207 | 1.00 | 0.00 | O |
| ATOM | 8714 | OH2 | WAT | W 532 | 39.879 | 38.867 | −3.565 | 1.00 | 0.00 | O |
| ATOM | 8715 | OH2 | WAT | W 533 | 39.730 | 96.389 | −28.114 | 1.00 | 0.00 | O |
| ATOM | 8716 | OH2 | WAT | W 534 | 36.679 | 35.119 | −15.776 | 1.00 | 0.00 | O |
| ATOM | 8717 | OH2 | WAT | W 535 | 34.980 | 45.884 | 33.331 | 1.00 | 0.00 | O |
| ATOM | 8718 | OH2 | WAT | W 536 | 55.738 | 78.083 | −1.633 | 1.00 | 0.00 | O |
| ATOM | 8719 | OH2 | WAT | W 537 | 69.975 | 62.220 | −0.091 | 1.00 | 0.00 | O |
| ATOM | 8720 | OH2 | WAT | W 538 | 46.089 | 74.493 | 22.711 | 1.00 | 0.00 | O |
| ATOM | 8721 | OH2 | WAT | W 539 | 48.479 | 46.074 | −23.173 | 1.00 | 0.00 | O |
| ATOM | 8722 | OH2 | WAT | W 540 | 60.426 | 92.218 | −28.568 | 1.00 | 0.00 | O |
| ATOM | 8723 | OH2 | WAT | W 541 | 37.691 | 62.372 | 35.386 | 1.00 | 0.00 | O |
| ATOM | 8724 | OH2 | WAT | W 542 | 29.219 | 63.275 | −37.448 | 1.00 | 0.00 | O |
| ATOM | 8725 | OH2 | WAT | W 543 | 61.269 | 48.104 | −31.683 | 1.00 | 0.00 | O |
| ATOM | 8726 | OH2 | WAT | W 544 | 61.987 | 83.540 | −38.118 | 1.00 | 0.00 | O |
| ATOM | 8727 | OH2 | WAT | W 545 | 62.916 | 42.910 | −20.939 | 1.00 | 0.00 | O |
| ATOM | 8728 | OH2 | WAT | W 546 | 53.462 | 44.400 | 22.714 | 1.00 | 0.00 | O |
| ATOM | 8729 | OH2 | WAT | W 547 | 30.820 | 34.783 | −9.453 | 1.00 | 0.00 | O |
| ATOM | 8730 | OH2 | WAT | W 548 | 29.478 | 31.624 | 21.760 | 1.00 | 0.00 | O |
| ATOM | 8731 | OH2 | WAT | W 549 | 78.634 | 69.005 | −12.213 | 1.00 | 0.00 | O |
| ATOM | 8732 | OH2 | WAT | W 550 | 31.453 | 69.262 | −45.544 | 1.00 | 0.00 | O |
| ATOM | 8733 | OH2 | WAT | W 551 | 73.440 | 82.530 | −21.355 | 1.00 | 0.00 | O |
| ATOM | 8734 | OH2 | WAT | W 552 | 47.357 | 71.312 | −23.900 | 1.00 | 0.00 | O |
| ATOM | 8735 | OH2 | WAT | W 553 | 20.487 | 38.004 | 12.047 | 1.00 | 0.00 | O |
| ATOM | 8736 | OH2 | WAT | W 554 | 35.053 | 95.226 | −32.231 | 1.00 | 0.00 | O |
| ATOM | 8737 | OH2 | WAT | W 555 | 17.080 | 41.539 | 9.158 | 1.00 | 0.00 | O |
| ATOM | 8738 | OH2 | WAT | W 556 | 20.621 | 80.362 | −33.678 | 1.00 | 0.00 | O |
| ATOM | 8739 | OH2 | WAT | W 557 | 49.081 | 92.026 | −24.071 | 1.00 | 0.00 | O |
| ATOM | 8740 | OH2 | WAT | W 558 | 43.730 | 44.890 | 19.299 | 1.00 | 0.00 | O |
| ATOM | 8741 | OH2 | WAT | W 559 | 21.202 | 35.666 | −5.127 | 1.00 | 0.00 | O |
| ATOM | 8742 | OH2 | WAT | W 560 | 65.011 | 88.072 | −24.218 | 1.00 | 0.00 | O |
| ATOM | 8743 | OH2 | WAT | W 561 | 46.925 | 53.987 | −21.419 | 1.00 | 0.00 | O |
| ATOM | 8744 | OH2 | WAT | W 562 | 71.377 | 63.278 | −27.101 | 1.00 | 0.00 | O |
| ATOM | 8745 | OH2 | WAT | W 563 | 20.022 | 35.601 | 25.719 | 1.00 | 0.00 | O |
| ATOM | 8746 | OH2 | WAT | W 564 | 59.362 | 87.224 | −45.669 | 1.00 | 0.00 | O |
| ATOM | 8747 | OH2 | WAT | W 565 | 51.846 | 34.237 | −7.781 | 1.00 | 0.00 | O |
| ATOM | 8748 | OH2 | WAT | W 566 | 56.174 | 79.375 | −15.045 | 1.00 | 0.00 | O |
| ATOM | 8749 | OH2 | WAT | W 567 | 28.897 | 70.037 | −39.456 | 1.00 | 0.00 | O |
| ATOM | 8750 | OH2 | WAT | W 568 | 45.132 | 83.415 | −43.264 | 1.00 | 0.00 | O |
| ATOM | 8751 | OH2 | WAT | W 569 | 29.704 | 81.723 | −42.811 | 1.00 | 0.00 | O |
| ATOM | 8752 | OH2 | WAT | W 570 | 28.566 | 90.812 | −42.631 | 1.00 | 0.00 | O |
| ATOM | 8753 | OH2 | WAT | W 571 | 19.650 | 84.825 | −28.456 | 1.00 | 0.00 | O |
| ATOM | 8754 | OH2 | WAT | W 572 | 37.818 | 97.411 | −30.646 | 1.00 | 0.00 | O |
| ATOM | 8755 | OH2 | WAT | W 573 | 40.603 | 44.569 | −31.615 | 1.00 | 0.00 | O |
| ATOM | 8756 | OH2 | WAT | W 574 | 21.065 | 41.634 | 35.090 | 1.00 | 0.00 | O |
| ATOM | 8757 | OH2 | WAT | W 575 | 39.341 | 30.547 | 13.180 | 1.00 | 0.00 | O |
| ATOM | 8758 | OH2 | WAT | W 576 | 62.232 | 85.898 | −23.043 | 1.00 | 0.00 | O |
| ATOM | 8759 | OH2 | WAT | W 577 | 31.093 | 68.494 | 14.062 | 1.00 | 0.00 | O |
| ATOM | 8760 | OH2 | WAT | W 578 | 28.012 | 67.466 | 33.703 | 1.00 | 0.00 | O |
| ATOM | 8761 | OH2 | WAT | W 579 | 14.919 | 63.617 | −25.796 | 1.00 | 0.00 | O |
| ATOM | 8762 | OH2 | WAT | W 580 | 29.777 | 72.345 | 12.305 | 1.00 | 0.00 | O |
| ATOM | 8763 | OH2 | WAT | W 581 | 61.150 | 93.195 | −38.571 | 1.00 | 0.00 | O |
| ATOM | 8764 | OH2 | WAT | W 582 | 59.323 | 62.309 | 25.744 | 1.00 | 0.00 | O |
| ATOM | 8765 | OH2 | WAT | W 583 | 51.213 | 79.510 | −1.669 | 1.00 | 0.00 | O |
| ATOM | 8766 | OH2 | WAT | W 584 | 21.364 | 60.549 | 26.762 | 1.00 | 0.00 | O |
| ATOM | 8767 | OH2 | WAT | W 585 | 59.433 | 91.512 | −35.102 | 1.00 | 0.00 | O |
| ATOM | 8768 | OH2 | WAT | W 586 | 24.269 | 60.848 | 35.990 | 1.00 | 0.00 | O |
| ATOM | 8769 | OH2 | WAT | W 587 | 11.450 | 62.002 | 0.100 | 1.00 | 0.00 | O |
| ATOM | 8770 | OH2 | WAT | W 588 | 55.171 | 80.702 | −19.348 | 1.00 | 0.00 | O |
| ATOM | 8771 | OH2 | WAT | W 589 | 16.962 | 74.562 | −28.650 | 1.00 | 0.00 | O |
| ATOM | 8772 | OH2 | WAT | W 590 | 40.725 | 40.685 | −21.417 | 1.00 | 0.00 | O |
| ATOM | 8773 | OH2 | WAT | W 591 | 28.192 | 68.552 | −46.100 | 1.00 | 0.00 | O |
| ATOM | 8774 | OH2 | WAT | W 592 | 40.020 | 35.502 | 11.339 | 1.00 | 0.00 | O |
| ATOM | 8775 | OH2 | WAT | W 593 | 19.044 | 80.664 | −11.850 | 1.00 | 0.00 | O |
| ATOM | 8776 | OH2 | WAT | W 594 | 64.962 | 80.057 | −8.837 | 1.00 | 0.00 | O |
| ATOM | 8777 | OH2 | WAT | W 595 | 72.971 | 43.614 | −12.173 | 1.00 | 0.00 | O |
| ATOM | 8778 | OH2 | WAT | W 596 | 12.870 | 74.892 | −19.225 | 1.00 | 0.00 | O |
| ATOM | 8779 | OH2 | WAT | W 597 | 57.091 | 75.709 | −2.819 | 1.00 | 0.00 | O |
| ATOM | 8780 | OH2 | WAT | W 598 | 20.941 | 58.551 | 37.845 | 1.00 | 0.00 | O |
| ATOM | 8781 | OH2 | WAT | W 599 | 50.724 | 77.524 | 2.143 | 1.00 | 0.00 | O |
| ATOM | 8782 | OH2 | WAT | W 600 | 28.397 | 45.580 | 40.744 | 1.00 | 0.00 | O |
| ATOM | 8783 | OH2 | WAT | W 601 | 23.038 | 36.989 | 13.347 | 1.00 | 0.00 | O |
| ATOM | 8784 | OH2 | WAT | W 602 | 33.273 | 39.694 | −19.124 | 1.00 | 0.00 | O |
| ATOM | 8785 | OH2 | WAT | W 603 | 33.739 | 49.423 | −15.040 | 1.00 | 0.00 | O |
| ATOM | 8786 | OH2 | WAT | W 604 | 29.464 | 88.981 | −26.114 | 1.00 | 0.00 | O |
| ATOM | 8787 | OH2 | WAT | W 605 | 37.103 | 33.836 | 5.763 | 1.00 | 0.00 | O |
| ATOM | 8788 | OH2 | WAT | W 606 | 71.868 | 62.856 | −18.090 | 1.00 | 0.00 | O |
| ATOM | 8789 | OH2 | WAT | W 607 | 34.298 | 70.277 | −44.850 | 1.00 | 0.00 | O |
| ATOM | 8790 | OH2 | WAT | W 608 | 64.246 | 77.356 | 0.371 | 1.00 | 0.00 | O |
| ATOM | 8791 | OH2 | WAT | W 609 | 35.765 | 55.988 | 8.553 | 1.00 | 0.00 | O |
| ATOM | 8792 | OH2 | WAT | W 610 | 30.746 | 51.320 | 39.591 | 1.00 | 0.00 | O |
| ATOM | 8793 | OH2 | WAT | W 611 | 54.247 | 58.047 | −13.809 | 1.00 | 0.00 | O |
| ATOM | 8794 | OH2 | WAT | W 612 | 21.033 | 54.593 | 46.942 | 1.00 | 0.00 | O |
| ATOM | 8795 | OH2 | WAT | W 613 | 14.544 | 50.681 | 0.941 | 1.00 | 0.00 | O |
| ATOM | 8796 | OH2 | WAT | W 614 | 25.361 | 91.705 | −32.972 | 1.00 | 0.00 | O |
| ATOM | 8797 | OH2 | WAT | W 615 | 73.166 | 71.394 | −29.179 | 1.00 | 0.00 | O |
| ATOM | 8798 | OH2 | WAT | W 616 | 55.692 | 38.636 | 0.977 | 1.00 | 0.00 | O |
| ATOM | 8799 | OH2 | WAT | W 617 | 37.473 | 84.217 | 4.454 | 1.00 | 0.00 | O |
| ATOM | 8800 | OH2 | WAT | W 618 | 45.668 | 55.250 | 10.276 | 1.00 | 0.00 | O |
| ATOM | 8801 | OH2 | WAT | W 619 | 67.054 | 81.349 | −11.203 | 1.00 | 0.00 | O |
| ATOM | 8802 | OH2 | WAT | W 620 | 40.531 | 89.320 | −19.530 | 1.00 | 0.00 | O |
| ATOM | 8803 | OH2 | WAT | W 621 | 43.788 | 56.725 | 33.520 | 1.00 | 0.00 | O |
| ATOM | 8804 | OH2 | WAT | W 622 | 56.284 | 50.275 | −30.442 | 1.00 | 0.00 | O |
| ATOM | 8805 | OH2 | WAT | W 623 | 63.547 | 57.533 | 26.047 | 1.00 | 0.00 | O |
| ATOM | 8806 | OH2 | WAT | W 624 | 62.152 | 48.417 | −20.968 | 1.00 | 0.00 | O |
| ATOM | 8807 | OH2 | WAT | W 625 | 62.865 | 59.678 | −22.138 | 1.00 | 0.00 | O |
| ATOM | 8808 | OH2 | WAT | W 626 | 39.807 | 77.878 | 13.615 | 1.00 | 0.00 | O |
| ATOM | 8809 | OH2 | WAT | W 627 | 26.705 | 92.002 | −27.583 | 1.00 | 0.00 | O |
| ATOM | 8810 | OH2 | WAT | W 628 | 13.511 | 70.306 | −9.673 | 1.00 | 0.00 | O |
| ATOM | 8811 | OH2 | WAT | W 629 | 60.650 | 79.062 | −41.480 | 1.00 | 0.00 | O |
| ATOM | 8812 | OH2 | WAT | W 630 | 39.200 | 43.459 | 26.617 | 1.00 | 0.00 | O |
| ATOM | 8813 | OH2 | WAT | W 631 | 12.021 | 71.041 | 2.447 | 1.00 | 0.00 | O |
| ATOM | 8814 | OH2 | WAT | W 632 | 36.432 | 62.728 | −38.343 | 1.00 | 0.00 | O |
| ATOM | 8815 | OH2 | WAT | W 633 | 76.004 | 83.239 | −25.233 | 1.00 | 0.00 | O |
| ATOM | 8816 | OH2 | WAT | W 634 | 37.691 | 80.755 | −0.580 | 1.00 | 0.00 | O |
| ATOM | 8817 | OH2 | WAT | W 635 | 47.388 | 53.701 | 35.727 | 1.00 | 0.00 | O |
| ATOM | 8818 | OH2 | WAT | W 636 | 48.724 | 97.982 | −33.771 | 1.00 | 0.00 | O |
| ATOM | 8819 | OH2 | WAT | W 637 | 63.284 | 75.071 | −45.697 | 1.00 | 0.00 | O |
| ATOM | 8820 | OH2 | WAT | W 638 | 60.036 | 71.911 | 0.281 | 1.00 | 0.00 | O |
| ATOM | 8821 | OH2 | WAT | W 639 | 40.994 | 48.115 | −29.333 | 1.00 | 0.00 | O |
| ATOM | 8822 | OH2 | WAT | W 640 | 55.304 | 40.303 | −12.656 | 1.00 | 0.00 | O |
| ATOM | 8823 | OH2 | WAT | W 641 | 75.278 | 84.083 | −22.808 | 1.00 | 0.00 | O |
| ATOM | 8824 | OH2 | WAT | W 642 | 63.429 | 52.129 | −0.436 | 1.00 | 0.00 | O |
| ATOM | 8825 | OH2 | WAT | W 643 | 37.171 | 36.676 | 19.220 | 1.00 | 0.00 | O |
| ATOM | 8826 | OH2 | WAT | W 644 | 57.798 | 36.026 | −2.366 | 1.00 | 0.00 | O |
| ATOM | 8827 | OH2 | WAT | W 645 | 23.216 | 48.896 | −36.160 | 1.00 | 0.00 | O |
| ATOM | 8828 | OH2 | WAT | W 646 | 18.051 | 71.467 | −29.826 | 1.00 | 0.00 | O |
| ATOM | 8829 | OH2 | WAT | W 647 | 30.822 | 40.388 | 38.149 | 1.00 | 0.00 | O |
| ATOM | 8830 | OH2 | WAT | W 648 | 27.605 | 50.308 | 23.225 | 1.00 | 0.00 | O |
| ATOM | 8831 | OH2 | WAT | W 649 | 30.597 | 47.814 | 40.348 | 1.00 | 0.00 | O |
| ATOM | 8832 | OH2 | WAT | W 650 | 59.960 | 52.971 | −30.664 | 1.00 | 0.00 | O |
| ATOM | 8833 | OH2 | WAT | W 651 | 44.799 | 40.674 | 13.217 | 1.00 | 0.00 | O |
| ATOM | 8834 | OH2 | WAT | W 652 | 34.017 | 47.888 | 36.072 | 1.00 | 0.00 | O |
| ATOM | 8835 | OH2 | WAT | W 653 | 27.187 | 82.319 | 3.172 | 1.00 | 0.00 | O |
| ATOM | 8836 | OH2 | WAT | W 654 | 58.515 | 92.353 | −42.111 | 1.00 | 0.00 | O |
| ATOM | 8837 | OH2 | WAT | W 655 | 49.126 | 52.288 | −37.796 | 1.00 | 0.00 | O |
| ATOM | 8838 | OH2 | WAT | W 656 | 53.313 | 71.104 | −16.923 | 1.00 | 0.00 | O |
| ATOM | 8839 | OH2 | WAT | W 657 | 57.589 | 62.825 | −15.540 | 1.00 | 0.00 | O |
| ATOM | 8840 | OH2 | WAT | W 658 | 28.352 | 44.446 | −29.609 | 1.00 | 0.00 | O |
| ATOM | 8841 | OH2 | WAT | W 659 | 49.081 | 49.407 | −38.302 | 1.00 | 0.00 | O |
| ATOM | 8842 | OH2 | WAT | W 660 | 35.575 | 77.711 | −43.096 | 1.00 | 0.00 | O |
| ATOM | 8843 | OH2 | WAT | W 661 | 9.831 | 58.785 | 17.833 | 1.00 | 0.00 | O |
| ATOM | 8844 | OH2 | WAT | W 662 | 61.038 | 45.561 | −3.765 | 1.00 | 0.00 | O |
| ATOM | 8845 | OH2 | WAT | W 663 | 65.482 | 78.986 | −5.781 | 1.00 | 0.00 | O |
| ATOM | 8846 | OH2 | WAT | W 664 | 7.036 | 58.315 | 14.701 | 1.00 | 0.00 | O |
| ATOM | 8847 | OH2 | WAT | W 665 | 49.623 | 50.858 | 32.493 | 1.00 | 0.00 | O |
| ATOM | 8848 | OH2 | WAT | W 666 | 18.541 | 62.487 | 22.934 | 1.00 | 0.00 | O |
| ATOM | 8849 | OH2 | WAT | W 667 | 30.423 | 34.190 | 21.123 | 1.00 | 0.00 | O |
| ATOM | 8850 | OH2 | WAT | W 668 | 17.479 | 45.675 | −14.263 | 1.00 | 0.00 | O |
| ATOM | 8851 | OH2 | WAT | W 669 | 28.774 | 55.333 | 39.968 | 1.00 | 0.00 | O |
| ATOM | 8852 | OH2 | WAT | W 670 | 50.805 | 60.075 | −8.931 | 1.00 | 0.00 | O |
| ATOM | 8853 | OH2 | WAT | W 671 | 43.865 | 63.225 | −28.668 | 1.00 | 0.00 | O |
| ATOM | 8854 | OH2 | WAT | W 672 | 38.137 | 82.016 | −31.368 | 1.00 | 0.00 | O |
| ATOM | 8855 | OH2 | WAT | W 673 | 32.333 | 39.224 | −4.953 | 1.00 | 0.00 | O |
| ATOM | 8856 | OH2 | WAT | W 674 | 60.357 | 61.090 | −18.144 | 1.00 | 0.00 | O |
| ATOM | 8857 | OH2 | WAT | W 675 | 63.176 | 58.695 | −1.449 | 1.00 | 0.00 | O |
| ATOM | 8858 | OH2 | WAT | W 676 | 60.718 | 61.111 | −3.563 | 1.00 | 0.00 | O |
| ATOM | 8859 | OH2 | WAT | W 677 | 46.652 | 74.662 | 7.739 | 1.00 | 0.00 | O |
| ATOM | 8860 | OH2 | WAT | W 678 | 54.971 | 57.680 | −0.492 | 1.00 | 0.00 | O |
| ATOM | 8861 | OH2 | WAT | W 679 | 30.170 | 75.107 | −29.270 | 1.00 | 0.00 | O |
| ATOM | 8862 | OH2 | WAT | W 680 | 58.915 | 56.852 | −1.451 | 1.00 | 0.00 | O |
| ATOM | 8863 | OH2 | WAT | W 681 | 58.699 | 52.771 | −0.065 | 1.00 | 0.00 | O |
| ATOM | 8864 | OH2 | WAT | W 682 | 63.032 | 61.194 | −4.945 | 1.00 | 0.00 | O |
| ATOM | 8865 | OH2 | WAT | W 683 | 27.869 | 48.760 | 12.114 | 1.00 | 0.00 | O |
| ATOM | 8866 | OH2 | WAT | W 684 | 66.585 | 56.380 | −20.256 | 1.00 | 0.00 | O |
| ATOM | 8867 | OH2 | WAT | W 685 | 19.828 | 46.907 | −14.377 | 1.00 | 0.00 | O |
| ATOM | 8868 | OH2 | WAT | W 686 | 38.637 | 69.412 | −38.139 | 1.00 | 0.00 | O |
| ATOM | 8869 | OH2 | WAT | W 687 | 33.760 | 60.433 | 23.248 | 1.00 | 0.00 | O |
| ATOM | 8870 | OH2 | WAT | W 688 | 39.618 | 55.795 | 3.003 | 1.00 | 0.00 | O |
| ATOM | 8871 | OH2 | WAT | W 689 | 24.685 | 61.653 | 9.124 | 1.00 | 0.00 | O |
| ATOM | 8872 | OH2 | WAT | W 690 | 16.854 | 57.352 | 13.005 | 1.00 | 0.00 | O |
| ATOM | 8873 | OH2 | WAT | W 691 | 39.367 | 69.367 | −35.449 | 1.00 | 0.00 | O |
| ATOM | 8874 | OH2 | WAT | W 692 | 49.897 | 67.815 | 4.117 | 1.00 | 0.00 | O |
| ATOM | 8875 | OH2 | WAT | W 693 | 27.764 | 53.504 | 20.003 | 1.00 | 0.00 | O |
| ATOM | 8876 | OH2 | WAT | W 694 | 29.601 | 83.195 | 2.383 | 1.00 | 0.00 | O |
| ATOM | 8877 | OH2 | WAT | W 695 | 28.124 | 91.130 | −25.262 | 1.00 | 0.00 | O |
| ATOM | 8878 | OH2 | WAT | W 696 | 46.063 | 77.857 | 9.463 | 1.00 | 0.00 | O |
| ATOM | 8879 | OH2 | WAT | W 697 | 12.616 | 49.439 | 17.771 | 1.00 | 0.00 | O |
| ATOM | 8880 | OH2 | WAT | W 698 | 81.174 | 68.674 | −11.422 | 1.00 | 0.00 | O |
| ATOM | 8881 | OH2 | WAT | W 699 | 41.447 | 44.919 | −6.967 | 1.00 | 0.00 | O |
| ATOM | 8882 | OH2 | WAT | W 700 | 45.661 | 67.945 | −27.578 | 1.00 | 0.00 | O |
| ATOM | 8883 | OH2 | WAT | W 701 | 14.270 | 50.705 | 24.207 | 1.00 | 0.00 | O |
| ATOM | 8884 | OH2 | WAT | W 702 | 67.411 | 46.627 | −1.397 | 1.00 | 0.00 | O |
| ATOM | 8885 | OH2 | WAT | W 703 | 9.073 | 50.490 | 8.141 | 1.00 | 0.00 | O |
| ATOM | 8886 | OH2 | WAT | W 704 | 48.445 | 40.966 | −1.167 | 1.00 | 0.00 | O |
| ATOM | 8887 | OH2 | WAT | W 705 | 17.965 | 74.633 | 3.936 | 1.00 | 0.00 | O |
| ATOM | 8888 | OH2 | WAT | W 706 | 44.849 | 55.258 | −18.439 | 1.00 | 0.00 | O |
| ATOM | 8889 | OH2 | WAT | W 707 | 83.509 | 66.541 | −15.446 | 1.00 | 0.00 | O |
| ATOM | 8890 | OH2 | WAT | W 708 | 48.836 | 69.219 | −36.853 | 1.00 | 0.00 | O |
| ATOM | 8891 | OH2 | WAT | W 709 | 51.740 | 70.392 | −38.053 | 1.00 | 0.00 | O |
| ATOM | 8892 | OH2 | WAT | W 710 | 29.670 | 72.514 | 34.334 | 1.00 | 0.00 | O |
| ATOM | 8893 | OH2 | WAT | W 711 | 37.551 | 61.588 | −32.581 | 1.00 | 0.00 | O |
| ATOM | 8894 | OH2 | WAT | W 712 | 57.368 | 49.094 | 21.323 | 1.00 | 0.00 | O |
| ATOM | 8895 | OH2 | WAT | W 713 | 42.954 | 59.615 | −31.428 | 1.00 | 0.00 | O |
| ATOM | 8896 | OH2 | WAT | W 714 | 32.002 | 34.493 | −3.286 | 1.00 | 0.00 | O |
| ATOM | 8897 | OH2 | WAT | W 715 | 37.059 | 59.240 | −33.582 | 1.00 | 0.00 | O |
| ATOM | 8898 | OH2 | WAT | W 716 | 61.585 | 59.248 | 14.620 | 1.00 | 0.00 | O |
| ATOM | 8899 | OH2 | WAT | W 717 | 40.232 | 56.409 | −18.976 | 1.00 | 0.00 | O |
| ATOM | 8900 | OH2 | WAT | W 718 | 47.491 | 87.686 | −44.193 | 1.00 | 0.00 | O |
| ATOM | 8901 | OH2 | WAT | W 719 | 61.744 | 76.681 | −10.413 | 1.00 | 0.00 | O |
| ATOM | 8902 | OH2 | WAT | W 720 | 79.481 | 50.956 | −11.440 | 1.00 | 0.00 | O |
| ATOM | 8903 | OH2 | WAT | W 721 | 37.564 | 51.745 | −18.481 | 1.00 | 0.00 | O |
| ATOM | 8904 | OH2 | WAT | W 722 | 25.700 | 52.288 | 13.042 | 1.00 | 0.00 | O |
| ATOM | 8905 | OH2 | WAT | W 723 | 69.556 | 64.080 | −28.582 | 1.00 | 0.00 | O |
| ATOM | 8906 | OH2 | WAT | W 724 | 72.922 | 66.794 | −10.027 | 1.00 | 0.00 | O |
| ATOM | 8907 | OH2 | WAT | W 725 | 42.702 | 56.558 | 2.475 | 1.00 | 0.00 | O |
| ATOM | 8908 | OH2 | WAT | W 726 | 56.024 | 65.323 | −36.107 | 1.00 | 0.00 | O |
| ATOM | 8909 | OH2 | WAT | W 727 | 17.862 | 66.380 | 28.012 | 1.00 | 0.00 | O |
| ATOM | 8910 | OH2 | WAT | W 728 | 11.723 | 63.777 | −16.969 | 1.00 | 0.00 | O |
| ATOM | 8911 | OH2 | WAT | W 729 | 35.297 | 86.417 | −17.891 | 1.00 | 0.00 | O |
| ATOM | 8912 | OH2 | WAT | W 730 | 9.747 | 60.412 | −20.401 | 1.00 | 0.00 | O |
| ATOM | 8913 | OH2 | WAT | W 731 | 11.666 | 48.540 | −2.453 | 1.00 | 0.00 | O |
| ATOM | 8914 | OH2 | WAT | W 732 | 40.620 | 57.107 | 31.023 | 1.00 | 0.00 | O |
| ATOM | 8915 | OH2 | WAT | W 733 | 39.781 | 53.162 | 36.395 | 1.00 | 0.00 | O |
| ATOM | 8916 | OH2 | WAT | W 734 | 49.828 | 47.378 | 28.831 | 1.00 | 0.00 | O |
| ATOM | 8917 | OH2 | WAT | W 735 | 24.121 | 34.445 | 15.342 | 1.00 | 0.00 | O |
| ATOM | 8918 | OH2 | WAT | W 736 | 59.484 | 51.068 | 24.851 | 1.00 | 0.00 | O |
| ATOM | 8919 | OH2 | WAT | W 737 | 38.048 | 83.338 | −1.088 | 1.00 | 0.00 | O |
| ATOM | 8920 | OH2 | WAT | W 738 | 13.039 | 51.659 | −12.221 | 1.00 | 0.00 | O |
| ATOM | 8921 | OH2 | WAT | W 739 | 48.491 | 50.816 | 35.090 | 1.00 | 0.00 | O |
| ATOM | 8922 | OH2 | WAT | W 740 | 48.024 | 41.657 | −5.539 | 1.00 | 0.00 | O |
| ATOM | 8923 | OH2 | WAT | W 741 | 22.233 | 40.924 | −11.345 | 1.00 | 0.00 | O |
| ATOM | 8924 | OH2 | WAT | W 742 | 16.130 | 36.463 | 16.265 | 1.00 | 0.00 | O |
| ATOM | 8925 | OH2 | WAT | W 743 | 22.459 | 68.166 | 20.245 | 1.00 | 0.00 | O |
| ATOM | 8926 | OH2 | WAT | W 744 | 72.044 | 47.327 | −17.973 | 1.00 | 0.00 | O |
| ATOM | 8927 | OH2 | WAT | W 745 | 40.850 | 85.379 | −11.971 | 1.00 | 0.00 | O |
| ATOM | 8928 | OH2 | WAT | W 746 | 56.754 | 46.779 | 15.696 | 1.00 | 0.00 | O |
| ATOM | 8929 | OH2 | WAT | W 747 | 51.912 | 64.114 | 24.561 | 1.00 | 0.00 | O |
| ATOM | 8930 | OH2 | WAT | W 748 | 56.583 | 58.561 | 1.343 | 1.00 | 0.00 | O |
| ATOM | 8931 | OH2 | WAT | W 749 | 57.375 | 58.590 | 5.505 | 1.00 | 0.00 | O |
| ATOM | 8932 | OH2 | WAT | W 750 | 75.112 | 64.404 | −16.409 | 1.00 | 0.00 | O |
| ATOM | 8933 | OH2 | WAT | W 751 | 14.677 | 70.800 | −23.130 | 1.00 | 0.00 | O |
| ATOM | 8934 | OH2 | WAT | W 752 | 12.928 | 52.332 | −15.040 | 1.00 | 0.00 | O |
| ATOM | 8935 | OH2 | WAT | W 753 | 12.990 | 62.106 | −25.014 | 1.00 | 0.00 | O |
| ATOM | 8936 | OH2 | WAT | W 754 | 23.320 | 87.353 | −32.623 | 1.00 | 0.00 | O |
| ATOM | 8937 | OH2 | WAT | W 755 | 66.467 | 81.834 | −14.275 | 1.00 | 0.00 | O |
| ATOM | 8938 | OH2 | WAT | W 756 | 47.246 | 61.563 | −28.821 | 1.00 | 0.00 | O |
| ATOM | 8939 | OH2 | WAT | W 757 | 52.548 | 71.145 | 5.916 | 1.00 | 0.00 | O |
| ATOM | 8940 | OH2 | WAT | W 758 | 40.996 | 56.024 | −33.206 | 1.00 | 0.00 | O |
| ATOM | 8941 | OH2 | WAT | W 759 | 39.620 | 39.909 | 19.999 | 1.00 | 0.00 | O |
| ATOM | 8942 | OH2 | WAT | W 760 | 44.780 | 55.346 | 36.103 | 1.00 | 0.00 | O |
| ATOM | 8943 | OH2 | WAT | W 761 | 13.957 | 62.533 | 14.560 | 1.00 | 0.00 | O |
| ATOM | 8944 | OH2 | WAT | W 762 | 39.951 | 39.546 | −0.340 | 1.00 | 0.00 | O |
| ATOM | 8945 | OH2 | WAT | W 763 | 32.665 | 69.632 | 22.105 | 1.00 | 0.00 | O |
| ATOM | 8946 | OH2 | WAT | W 764 | 42.544 | 43.608 | −11.932 | 1.00 | 0.00 | O |
| ATOM | 8947 | OH2 | WAT | W 765 | 26.986 | 41.823 | −27.491 | 1.00 | 0.00 | O |
| ATOM | 8948 | OH2 | WAT | W 766 | 19.699 | 41.810 | −5.043 | 1.00 | 0.00 | O |
| ATOM | 8949 | OH2 | WAT | W 767 | 13.702 | 60.846 | 7.536 | 1.00 | 0.00 | O |
| ATOM | 8950 | OH2 | WAT | W 768 | 46.607 | 45.815 | 11.519 | 1.00 | 0.00 | O |
| ATOM | 8951 | OH2 | WAT | W 769 | 43.014 | 68.693 | −30.021 | 1.00 | 0.00 | O |
| ATOM | 8952 | OH2 | WAT | W 770 | 24.369 | 54.092 | −8.366 | 1.00 | 0.00 | O |
| ATOM | 8953 | OH2 | WAT | W 771 | 47.715 | 70.197 | −16.593 | 1.00 | 0.00 | O |
| ATOM | 8954 | OH2 | WAT | W 772 | 58.809 | 93.869 | −27.437 | 1.00 | 0.00 | O |
| ATOM | 8955 | OH2 | WAT | W 773 | 31.147 | 79.134 | −42.936 | 1.00 | 0.00 | O |
| ATOM | 8956 | OH2 | WAT | W 774 | 22.049 | 42.744 | −13.274 | 1.00 | 0.00 | O |
| ATOM | 8957 | OH2 | WAT | W 775 | 52.881 | 92.375 | −23.179 | 1.00 | 0.00 | O |
| ATOM | 8958 | OH2 | WAT | W 776 | 60.191 | 51.118 | 20.150 | 1.00 | 0.00 | O |
| ATOM | 8959 | OH2 | WAT | W 777 | 60.990 | 56.042 | 1.994 | 1.00 | 0.00 | O |
| ATOM | 8960 | OH2 | WAT | W 778 | 19.496 | 58.666 | −36.837 | 1.00 | 0.00 | O |
| ATOM | 8961 | OH2 | WAT | W 779 | 30.066 | 50.044 | 12.371 | 1.00 | 0.00 | O |
| ATOM | 8962 | OH2 | WAT | W 780 | 26.332 | 66.843 | 19.829 | 1.00 | 0.00 | O |
| ATOM | 8963 | OH2 | WAT | W 781 | 12.053 | 41.630 | 19.820 | 1.00 | 0.00 | O |
| ATOM | 8964 | OH2 | WAT | W 782 | 69.448 | 77.276 | −34.137 | 1.00 | 0.00 | O |
| ATOM | 8965 | OH2 | WAT | W 783 | 16.598 | 43.024 | −2.692 | 1.00 | 0.00 | O |
| ATOM | 8966 | OH2 | WAT | W 784 | 35.778 | 59.983 | −37.738 | 1.00 | 0.00 | O |
| ATOM | 8967 | OH2 | WAT | W 785 | 33.877 | 66.335 | −42.421 | 1.00 | 0.00 | O |
| ATOM | 8968 | OH2 | WAT | W 786 | 57.155 | 36.347 | −14.319 | 1.00 | 0.00 | O |
| ATOM | 8969 | OH2 | WAT | W 787 | 37.130 | 37.634 | 2.914 | 1.00 | 0.00 | O |
| ATOM | 8970 | OH2 | WAT | W 788 | 51.229 | 66.915 | 8.358 | 1.00 | 0.00 | O |
| ATOM | 8971 | OH2 | WAT | W 789 | 10.809 | 51.683 | 21.412 | 1.00 | 0.00 | O |
| ATOM | 8972 | OH2 | WAT | W 790 | 30.272 | 87.456 | −15.075 | 1.00 | 0.00 | O |
| ATOM | 8973 | OH2 | WAT | W 791 | 42.000 | 55.040 | −17.314 | 1.00 | 0.00 | O |
| ATOM | 8974 | OH2 | WAT | W 792 | 48.599 | 76.287 | 8.352 | 1.00 | 0.00 | O |
| ATOM | 8975 | OH2 | WAT | W 793 | 29.556 | 75.889 | 10.811 | 1.00 | 0.00 | O |
| ATOM | 8976 | OH2 | WAT | W 794 | 42.983 | 98.265 | −37.032 | 1.00 | 0.00 | O |
| ATOM | 8977 | OH2 | WAT | W 795 | 23.457 | 66.161 | 9.814 | 1.00 | 0.00 | O |
| ATOM | 8978 | OH2 | WAT | W 796 | 64.821 | 79.071 | −1.641 | 1.00 | 0.00 | O |
| ATOM | 8979 | OH2 | WAT | W 797 | 43.476 | 40.517 | −22.898 | 1.00 | 0.00 | O |
| ATOM | 8980 | OH2 | WAT | W 798 | 59.380 | 49.094 | 2.175 | 1.00 | 0.00 | O |
| ATOM | 8981 | OH2 | WAT | W 799 | 68.965 | 41.765 | −17.204 | 1.00 | 0.00 | O |
| ATOM | 8982 | OH2 | WAT | W 800 | 24.786 | 71.958 | −39.859 | 1.00 | 0.00 | O |
| ATOM | 8983 | OH2 | WAT | W 801 | 23.791 | 48.577 | −24.248 | 1.00 | 0.00 | O |
| ATOM | 8984 | OH2 | WAT | W 802 | 46.992 | 68.200 | −24.723 | 1.00 | 0.00 | O |
| ATOM | 8985 | OH2 | WAT | W 803 | 53.469 | 53.184 | −29.954 | 1.00 | 0.00 | O |
| ATOM | 8986 | OH2 | WAT | W 804 | 24.847 | 34.448 | 35.644 | 1.00 | 0.00 | O |
| ATOM | 8987 | OH2 | WAT | W 805 | 13.398 | 52.665 | 25.851 | 1.00 | 0.00 | O |
| ATOM | 8988 | OH2 | WAT | W 806 | 51.565 | 44.643 | 2.883 | 1.00 | 0.00 | O |
| ATOM | 8989 | OH2 | WAT | W 807 | 21.403 | 55.666 | 39.696 | 1.00 | 0.00 | O |
| ATOM | 8990 | OH2 | WAT | W 808 | 64.124 | 70.731 | −5.187 | 1.00 | 0.00 | O |
| ATOM | 8991 | OH2 | WAT | W 809 | 46.964 | 89.865 | −23.190 | 1.00 | 0.00 | O |
| ATOM | 8992 | OH2 | WAT | W 810 | 8.004 | 53.162 | −7.590 | 1.00 | 0.00 | O |
| ATOM | 8993 | OH2 | WAT | W 811 | 22.178 | 80.917 | −42.185 | 1.00 | 0.00 | O |
| ATOM | 8994 | OH2 | WAT | W 812 | 63.791 | 65.738 | −30.505 | 1.00 | 0.00 | O |
| ATOM | 8995 | OH2 | WAT | W 813 | 18.344 | 48.705 | −19.772 | 1.00 | 0.00 | O |
| ATOM | 8996 | OH2 | WAT | W 814 | 59.393 | 76.939 | −4.266 | 1.00 | 0.00 | O |
| ATOM | 8997 | OH2 | WAT | W 815 | 21.046 | 84.137 | −12.343 | 1.00 | 0.00 | O |
| ATOM | 8998 | OH2 | WAT | W 816 | 55.637 | 67.379 | 13.249 | 1.00 | 0.00 | O |
| ATOM | 8999 | OH2 | WAT | W 817 | 19.129 | 52.421 | −31.535 | 1.00 | 0.00 | O |
| ATOM | 9000 | OH2 | WAT | W 818 | 67.310 | 85.719 | −35.909 | 1.00 | 0.00 | O |
| ATOM | 9001 | OH2 | WAT | W 819 | 29.648 | 75.153 | 22.302 | 1.00 | 0.00 | O |
| ATOM | 9002 | OH2 | WAT | W 820 | 32.734 | 84.320 | 0.358 | 1.00 | 0.00 | O |
| ATOM | 9003 | OH2 | WAT | W 821 | 45.616 | 65.480 | −28.642 | 1.00 | 0.00 | O |
| ATOM | 9004 | OH2 | WAT | W 822 | 12.769 | 62.208 | −2.408 | 1.00 | 0.00 | O |
| ATOM | 9005 | OH2 | WAT | W 823 | 25.815 | 63.461 | 13.819 | 1.00 | 0.00 | O |
| ATOM | 9006 | OH2 | WAT | W 824 | 28.537 | 35.024 | 32.178 | 1.00 | 0.00 | O |
| ATOM | 9007 | OH2 | WAT | W 825 | 36.003 | 68.685 | 23.484 | 1.00 | 0.00 | O |
| ATOM | 9008 | OH2 | WAT | W 826 | 31.941 | 33.882 | 17.234 | 1.00 | 0.00 | O |
| ATOM | 9009 | OH2 | WAT | W 827 | 41.632 | 84.306 | −2.386 | 1.00 | 0.00 | O |
| ATOM | 9010 | OH2 | WAT | W 828 | 40.025 | 100.327 | −31.898 | 1.00 | 0.00 | O |
| ATOM | 9011 | OH2 | WAT | W 829 | 28.679 | 63.614 | 10.610 | 1.00 | 0.00 | O |
| ATOM | 9012 | OH2 | WAT | W 830 | 54.710 | 82.573 | −44.566 | 1.00 | 0.00 | O |
| ATOM | 9013 | OH2 | WAT | W 831 | 69.910 | 53.740 | −10.499 | 1.00 | 0.00 | O |
| ATOM | 9014 | OH2 | WAT | W 832 | 36.929 | 79.493 | −2.515 | 1.00 | 0.00 | O |
| ATOM | 9015 | OH2 | WAT | W 833 | 12.241 | 64.675 | −12.654 | 1.00 | 0.00 | O |
| ATOM | 9016 | OH2 | WAT | W 834 | 39.116 | 50.345 | 36.275 | 1.00 | 0.00 | O |
| ATOM | 9017 | OH2 | WAT | W 835 | 27.945 | 44.812 | 38.120 | 1.00 | 0.00 | O |
| ATOM | 9018 | OH2 | WAT | W 836 | 27.807 | 38.775 | −22.548 | 1.00 | 0.00 | O |
| ATOM | 9019 | OH2 | WAT | W 837 | 72.318 | 95.802 | −31.265 | 1.00 | 0.00 | O |
| ATOM | 9020 | OH2 | WAT | W 838 | 76.337 | 47.098 | −11.656 | 1.00 | 0.00 | O |
| ATOM | 9021 | OH2 | WAT | W 839 | 21.476 | 49.083 | −38.816 | 1.00 | 0.00 | O |
| ATOM | 9022 | OH2 | WAT | W 840 | 23.077 | 75.197 | −30.035 | 1.00 | 0.00 | O |
| ATOM | 9023 | OH2 | WAT | W 841 | 21.007 | 31.697 | 31.656 | 1.00 | 0.00 | O |
| ATOM | 9024 | OH2 | WAT | W 842 | 29.496 | 42.250 | −29.768 | 1.00 | 0.00 | O |
| ATOM | 9025 | OH2 | WAT | W 843 | 42.674 | 47.359 | −27.307 | 1.00 | 0.00 | O |
| ATOM | 9026 | OH2 | WAT | W 844 | 55.875 | 51.903 | 27.280 | 1.00 | 0.00 | O |
| ATOM | 9027 | OH2 | WAT | W 845 | 31.320 | 58.788 | −40.699 | 1.00 | 0.00 | O |
| ATOM | 9028 | OH2 | WAT | W 846 | 18.413 | 43.157 | 27.335 | 1.00 | 0.00 | O |
| ATOM | 9029 | OH2 | WAT | W 847 | 62.202 | 81.779 | −43.989 | 1.00 | 0.00 | O |
| ATOM | 9030 | OH2 | WAT | W 848 | 7.598 | 54.603 | 17.501 | 1.00 | 0.00 | O |
| ATOM | 9031 | OH2 | WAT | W 849 | 18.093 | 41.894 | 25.173 | 1.00 | 0.00 | O |
| ATOM | 9032 | OH2 | WAT | W 850 | 27.410 | 82.732 | 5.773 | 1.00 | 0.00 | O |
| ATOM | 9033 | OH2 | WAT | W 851 | 38.754 | 53.282 | 1.951 | 1.00 | 0.00 | O |
| ATOM | 9034 | OH2 | WAT | W 852 | 11.935 | 67.433 | 2.649 | 1.00 | 0.00 | O |
| ATOM | 9035 | OH2 | WAT | W 853 | 4.365 | 56.736 | −6.558 | 1.00 | 0.00 | O |
| ATOM | 9036 | OH2 | WAT | W 854 | 22.251 | 77.051 | 8.631 | 1.00 | 0.00 | O |
| ATOM | 9037 | OH2 | WAT | W 855 | 25.871 | 88.232 | −42.851 | 1.00 | 0.00 | O |
| ATOM | 9038 | OH2 | WAT | W 856 | 10.526 | 62.685 | −12.061 | 1.00 | 0.00 | O |
| ATOM | 9039 | OH2 | WAT | W 857 | 44.547 | 72.288 | −44.586 | 1.00 | 0.00 | O |
| ATOM | 9040 | OH2 | WAT | W 858 | 37.290 | 40.031 | 26.372 | 1.00 | 0.00 | O |
| ATOM | 9041 | OH2 | WAT | W 859 | 23.413 | 89.021 | −37.997 | 1.00 | 0.00 | O |
| ATOM | 9042 | OH2 | WAT | W 860 | 70.125 | 62.786 | −24.288 | 1.00 | 0.00 | O |
| ATOM | 9043 | OH2 | WAT | W 861 | 23.006 | 64.510 | 18.349 | 1.00 | 0.00 | O |
| ATOM | 9044 | OH2 | WAT | W 862 | 67.072 | 38.620 | −9.608 | 1.00 | 0.00 | O |
| ATOM | 9045 | OH2 | WAT | W 863 | 65.525 | 87.848 | −41.036 | 1.00 | 0.00 | O |
| ATOM | 9046 | OH2 | WAT | W 864 | 13.974 | 43.632 | 28.969 | 1.00 | 0.00 | O |
| ATOM | 9047 | OH2 | WAT | W 865 | 13.517 | 48.343 | 1.249 | 1.00 | 0.00 | O |
| ATOM | 9048 | OH2 | WAT | W 866 | 55.479 | 69.588 | −37.978 | 1.00 | 0.00 | O |
| ATOM | 9049 | OH2 | WAT | W 867 | 26.978 | 47.633 | −23.933 | 1.00 | 0.00 | O |
| ATOM | 9050 | OH2 | WAT | W 868 | 69.691 | 82.317 | −33.913 | 1.00 | 0.00 | O |
| ATOM | 9051 | OH2 | WAT | W 869 | 12.132 | 62.283 | 22.698 | 1.00 | 0.00 | O |
| ATOM | 9052 | OH2 | WAT | W 870 | 30.805 | 29.810 | 5.462 | 1.00 | 0.00 | O |
| ATOM | 9053 | OH2 | WAT | W 871 | 19.362 | 36.440 | 22.388 | 1.00 | 0.00 | O |
| ATOM | 9054 | OH2 | WAT | W 872 | 27.607 | 86.561 | −14.749 | 1.00 | 0.00 | O |
| ATOM | 9055 | OH2 | WAT | W 873 | 66.284 | 50.407 | 0.526 | 1.00 | 0.00 | O |
| ATOM | 9056 | OH2 | WAT | W 874 | 28.556 | 64.834 | 14.855 | 1.00 | 0.00 | O |
| ATOM | 9057 | OH2 | WAT | W 875 | 52.010 | 60.249 | 28.130 | 1.00 | 0.00 | O |
| ATOM | 9058 | OH2 | WAT | W 876 | 52.467 | 72.620 | 18.424 | 1.00 | 0.00 | O |
| ATOM | 9059 | OH2 | WAT | W 877 | 47.028 | 75.056 | −26.303 | 1.00 | 0.00 | O |
| ATOM | 9060 | OH2 | WAT | W 878 | 65.388 | 81.563 | −4.616 | 1.00 | 0.00 | O |
| ATOM | 9061 | OH2 | WAT | W 879 | 48.555 | 72.298 | −26.138 | 1.00 | 0.00 | O |
| ATOM | 9062 | OH2 | WAT | W 880 | 59.805 | 68.884 | −38.756 | 1.00 | 0.00 | O |
| ATOM | 9063 | OH2 | WAT | W 881 | 22.839 | 85.133 | −16.960 | 1.00 | 0.00 | O |
| ATOM | 9064 | OH2 | WAT | W 882 | 25.891 | 76.368 | −38.719 | 1.00 | 0.00 | O |
| ATOM | 9065 | OH2 | WAT | W 883 | 20.306 | 74.098 | −29.005 | 1.00 | 0.00 | O |
| ATOM | 9066 | OH2 | WAT | W 884 | 59.313 | 55.625 | 27.048 | 1.00 | 0.00 | O |
| ATOM | 9067 | OH2 | WAT | W 885 | 44.374 | 87.552 | −15.607 | 1.00 | 0.00 | O |
| ATOM | 9068 | OH2 | WAT | W 886 | 63.968 | 77.026 | −40.510 | 1.00 | 0.00 | O |
| ATOM | 9069 | OH2 | WAT | W 887 | 14.193 | 58.389 | 16.309 | 1.00 | 0.00 | O |
| ATOM | 9070 | OH2 | WAT | W 888 | 13.991 | 57.800 | 18.963 | 1.00 | 0.00 | O |
| ATOM | 9071 | OH2 | WAT | W 889 | 24.433 | 63.725 | 11.590 | 1.00 | 0.00 | O |
| ATOM | 9072 | OH2 | WAT | W 890 | 28.173 | 39.815 | 11.264 | 1.00 | 0.00 | O |
| ATOM | 9073 | OH2 | WAT | W 891 | 28.969 | 82.087 | −35.700 | 1.00 | 0.00 | O |
| ATOM | 9074 | OH2 | WAT | W 892 | 12.334 | 55.892 | −15.106 | 1.00 | 0.00 | O |
| ATOM | 9075 | OH2 | WAT | W 893 | 81.492 | 67.210 | −13.715 | 1.00 | 0.00 | O |
| ATOM | 9076 | OH2 | WAT | W 894 | 58.943 | 58.063 | 0.989 | 1.00 | 0.00 | O |
| ATOM | 9077 | OH2 | WAT | W 895 | 49.240 | 68.478 | 6.745 | 1.00 | 0.00 | O |
| ATOM | 9078 | OH2 | WAT | W 896 | 68.453 | 79.691 | −28.505 | 1.00 | 0.00 | O |
| ATOM | 9079 | OH2 | WAT | W 897 | 26.063 | 32.733 | 28.695 | 1.00 | 0.00 | O |
| ATOM | 9080 | OH2 | WAT | W 898 | 32.825 | 69.554 | 24.592 | 1.00 | 0.00 | O |
| ATOM | 9081 | OH2 | WAT | W 899 | 28.004 | 102.786 | −19.159 | 1.00 | 0.00 | O |
| ATOM | 9082 | OH2 | WAT | W 900 | 71.706 | 73.979 | −29.110 | 1.00 | 0.00 | O |
| ATOM | 9083 | OH2 | WAT | W 901 | 79.308 | 50.637 | −8.873 | 1.00 | 0.00 | O |
| ATOM | 9084 | OH2 | WAT | W 902 | 37.119 | 83.812 | −3.965 | 1.00 | 0.00 | O |
| ATOM | 9085 | OH2 | WAT | W 903 | 59.380 | 52.104 | 2.479 | 1.00 | 0.00 | O |
| ATOM | 9086 | OH2 | WAT | W 904 | 19.831 | 84.498 | −18.520 | 1.00 | 0.00 | O |
| ATOM | 9087 | OH2 | WAT | W 905 | 43.800 | 79.352 | −21.525 | 1.00 | 0.00 | O |
| ATOM | 9088 | OH2 | WAT | W 906 | 57.934 | 52.938 | 26.177 | 1.00 | 0.00 | O |
| ATOM | 9089 | OH2 | WAT | W 907 | 16.972 | 57.508 | −36.393 | 1.00 | 0.00 | O |
| ATOM | 9090 | OH2 | WAT | W 908 | 46.716 | 42.927 | 9.598 | 1.00 | 0.00 | O |
| ATOM | 9091 | OH2 | WAT | W 909 | 27.293 | 72.028 | 33.949 | 1.00 | 0.00 | O |
| ATOM | 9092 | OH2 | WAT | W 910 | 7.399 | 54.460 | −10.010 | 1.00 | 0.00 | O |
| ATOM | 9093 | OH2 | WAT | W 911 | 24.567 | 52.467 | 43.509 | 1.00 | 0.00 | O |
| ATOM | 9094 | OH2 | WAT | W 912 | 49.872 | 47.054 | −30.505 | 1.00 | 0.00 | O |
| ATOM | 9095 | OH2 | WAT | W 913 | 42.648 | 78.827 | −29.498 | 1.00 | 0.00 | O |
| ATOM | 9096 | OH2 | WAT | W 914 | 27.560 | 88.968 | −12.451 | 1.00 | 0.00 | O |
| ATOM | 9097 | OH2 | WAT | W 915 | 56.665 | 90.188 | −43.623 | 1.00 | 0.00 | O |
| ATOM | 9098 | OH2 | WAT | W 916 | 13.988 | 44.952 | 26.828 | 1.00 | 0.00 | O |
| ATOM | 9099 | OH2 | WAT | W 917 | 69.599 | 84.021 | −36.023 | 1.00 | 0.00 | O |
| ATOM | 9100 | OH2 | WAT | W 918 | 58.001 | 93.501 | −25.084 | 1.00 | 0.00 | O |
| ATOM | 9101 | OH2 | WAT | W 919 | 64.750 | 70.415 | −8.067 | 1.00 | 0.00 | O |
| ATOM | 9102 | OH2 | WAT | W 920 | 46.080 | 39.915 | −0.024 | 1.00 | 0.00 | O |
| ATOM | 9103 | OH2 | WAT | W 921 | 37.037 | 37.401 | 24.945 | 1.00 | 0.00 | O |
| ATOM | 9104 | OH2 | WAT | W 922 | 52.328 | 68.609 | 4.799 | 1.00 | 0.00 | O |
| ATOM | 9105 | OH2 | WAT | W 923 | 17.944 | 51.019 | 38.085 | 1.00 | 0.00 | O |
| ATOM | 9106 | OH2 | WAT | W 924 | 15.329 | 77.248 | −13.725 | 1.00 | 0.00 | O |
| ATOM | 9107 | OH2 | WAT | W 925 | 20.080 | 39.386 | −12.345 | 1.00 | 0.00 | O |
| ATOM | 9108 | OH2 | WAT | W 926 | 49.590 | 45.658 | 30.987 | 1.00 | 0.00 | O |
| ATOM | 9109 | OH2 | WAT | W 927 | 46.966 | 72.548 | −17.619 | 1.00 | 0.00 | O |
| ATOM | 9110 | OH2 | WAT | W 928 | 70.530 | 78.324 | −25.996 | 1.00 | 0.00 | O |
| ATOM | 9111 | OH2 | WAT | W 929 | 58.528 | 52.925 | 4.642 | 1.00 | 0.00 | O |
| ATOM | 9112 | OH2 | WAT | W 930 | 35.511 | 55.482 | −34.790 | 1.00 | 0.00 | O |
| ATOM | 9113 | OH2 | WAT | W 931 | 41.566 | 82.880 | −28.827 | 1.00 | 0.00 | O |
| ATOM | 9114 | OH2 | WAT | W 932 | 48.139 | 66.187 | 26.918 | 1.00 | 0.00 | O |
| ATOM | 9115 | OH2 | WAT | W 933 | 27.688 | 67.687 | 11.673 | 1.00 | 0.00 | O |
| ATOM | 9116 | OH2 | WAT | W 934 | 39.791 | 81.980 | −21.883 | 1.00 | 0.00 | O |
| ATOM | 9117 | OH2 | WAT | W 935 | 22.231 | 65.784 | 32.283 | 1.00 | 0.00 | O |
| ATOM | 9118 | OH2 | WAT | W 936 | 58.785 | 48.756 | 18.929 | 1.00 | 0.00 | O |
| ATOM | 9119 | OH2 | WAT | W 937 | 31.846 | 80.989 | 7.175 | 1.00 | 0.00 | O |
| ATOM | 9120 | OH2 | WAT | W 938 | 50.357 | 45.000 | −24.797 | 1.00 | 0.00 | O |
| ATOM | 9121 | OH2 | WAT | W 939 | 62.512 | 60.777 | 0.439 | 1.00 | 0.00 | O |
| ATOM | 9122 | OH2 | WAT | W 940 | 67.855 | 75.395 | −35.094 | 1.00 | 0.00 | O |
| ATOM | 9123 | OH2 | WAT | W 941 | 23.146 | 39.423 | −15.577 | 1.00 | 0.00 | O |
| ATOM | 9124 | OH2 | WAT | W 942 | 35.988 | 94.020 | −29.930 | 1.00 | 0.00 | O |
| ATOM | 9125 | OH2 | WAT | W 943 | 74.007 | 80.191 | −11.042 | 1.00 | 0.00 | O |
| ATOM | 9126 | OH2 | WAT | W 944 | 41.099 | 81.809 | −3.455 | 1.00 | 0.00 | O |
| ATOM | 9127 | OH2 | WAT | W 945 | 13.012 | 62.985 | 12.102 | 1.00 | 0.00 | O |
| ATOM | 9128 | OH2 | WAT | W 946 | 56.731 | 76.091 | 0.096 | 1.00 | 0.00 | O |
| ATOM | 9129 | OH2 | WAT | W 947 | 74.305 | 52.098 | 1.514 | 1.00 | 0.00 | O |
| ATOM | 9130 | OH2 | WAT | W 948 | 22.012 | 61.473 | −34.863 | 1.00 | 0.00 | O |
| ATOM | 9131 | OH2 | WAT | W 949 | 77.405 | 65.439 | −15.062 | 1.00 | 0.00 | O |
| ATOM | 9132 | OH2 | WAT | W 950 | 17.698 | 41.847 | 32.112 | 1.00 | 0.00 | O |
| ATOM | 9133 | OH2 | WAT | W 951 | 10.249 | 63.069 | −15.024 | 1.00 | 0.00 | O |
| ATOM | 9134 | OH2 | WAT | W 952 | 45.901 | 79.927 | 4.907 | 1.00 | 0.00 | O |
| ATOM | 9135 | OH2 | WAT | W 953 | 14.666 | 46.936 | 32.185 | 1.00 | 0.00 | O |
| ATOM | 9136 | OH2 | WAT | W 954 | 34.450 | 77.326 | 12.201 | 1.00 | 0.00 | O |
| ATOM | 9137 | OH2 | WAT | W 955 | 47.528 | 40.258 | 5.987 | 1.00 | 0.00 | O |
| ATOM | 9138 | OH2 | WAT | W 956 | 18.746 | 39.516 | −2.924 | 1.00 | 0.00 | O |
| ATOM | 9139 | OH2 | WAT | W 957 | 25.892 | 59.095 | −38.859 | 1.00 | 0.00 | O |
| ATOM | 9140 | OH2 | WAT | W 958 | 10.667 | 57.925 | −20.958 | 1.00 | 0.00 | O |
| ATOM | 9141 | OH2 | WAT | W 959 | 8.868 | 56.882 | 16.385 | 1.00 | 0.00 | O |
| ATOM | 9142 | OH2 | WAT | W 960 | 78.132 | 72.243 | −10.244 | 1.00 | 0.00 | O |
| ATOM | 9143 | OH2 | WAT | W 961 | 23.897 | 82.874 | −3.971 | 1.00 | 0.00 | O |
| ATOM | 9144 | OH2 | WAT | W 962 | 44.484 | 40.987 | 7.504 | 1.00 | 0.00 | O |
| ATOM | 9145 | OH2 | WAT | W 963 | 35.423 | 62.467 | 33.988 | 1.00 | 0.00 | O |
| ATOM | 9146 | OH2 | WAT | W 964 | 27.265 | 90.075 | −31.712 | 1.00 | 0.00 | O |
| ATOM | 9147 | OH2 | WAT | W 965 | 74.755 | 77.389 | −20.721 | 1.00 | 0.00 | O |
| ATOM | 9148 | OH2 | WAT | W 966 | 49.089 | 42.037 | 8.287 | 1.00 | 0.00 | O |
| ATOM | 9149 | OH2 | WAT | W 967 | 44.887 | 92.912 | −25.033 | 1.00 | 0.00 | O |
| ATOM | 9150 | OH2 | WAT | W 968 | 52.465 | 49.136 | 25.639 | 1.00 | 0.00 | O |
| ATOM | 9151 | OH2 | WAT | W 969 | 9.657 | 53.649 | −12.165 | 1.00 | 0.00 | O |
| ATOM | 9152 | OH2 | WAT | W 970 | 58.698 | 51.115 | 14.502 | 1.00 | 0.00 | O |
| ATOM | 9153 | OH2 | WAT | W 971 | 51.871 | 50.475 | 27.678 | 1.00 | 0.00 | O |
| ATOM | 9154 | OH2 | WAT | W 972 | 25.018 | 89.096 | −25.145 | 1.00 | 0.00 | O |
| ATOM | 9155 | OH2 | WAT | W 973 | 7.496 | 58.258 | 0.195 | 1.00 | 0.00 | O |
| ATOM | 9156 | OH2 | WAT | W 974 | 11.522 | 58.486 | 19.993 | 1.00 | 0.00 | O |
| ATOM | 9157 | OH2 | WAT | W 975 | 18.602 | 60.933 | −37.788 | 1.00 | 0.00 | O |
| ATOM | 9158 | OH2 | WAT | W 976 | 46.005 | 60.116 | −31.276 | 1.00 | 0.00 | O |
| ATOM | 9159 | OH2 | WAT | W 977 | 5.372 | 57.741 | 6.747 | 1.00 | 0.00 | O |
| ATOM | 9160 | OH2 | WAT | W 978 | 13.334 | 66.476 | −11.620 | 1.00 | 0.00 | O |
| ATOM | 9161 | OH2 | WAT | W 979 | 47.210 | 81.653 | −17.119 | 1.00 | 0.00 | O |
| ATOM | 9162 | OH2 | WAT | W 981 | 29.419 | 65.681 | 12.431 | 1.00 | 0.00 | O |
| ATOM | 9163 | OH2 | WAT | W 982 | 28.576 | 69.917 | 12.812 | 1.00 | 0.00 | O |
| ATOM | 9164 | OH2 | WAT | W 983 | 33.300 | 68.977 | 16.903 | 1.00 | 0.00 | O |
| ATOM | 9165 | OH2 | WAT | W 984 | 26.458 | 67.157 | 9.398 | 1.00 | 0.00 | O |
| ATOM | 9166 | OH2 | WAT | W 985 | 32.012 | 71.165 | 13.558 | 1.00 | 0.00 | O |
| ATOM | 9167 | OH2 | WAT | W 986 | 25.611 | 66.324 | 17.380 | 1.00 | 0.00 | O |
| ATOM | 9168 | C1 | NAG | C 1 | 58.306 | 45.038 | 12.884 | 1.00 | 0.00 | C |
| ATOM | 9169 | C2 | NAG | C 1 | 59.529 | 44.683 | 13.738 | 1.00 | 0.00 | C |
| ATOM | 9170 | N2 | NAG | C 1 | 60.626 | 45.582 | 13.432 | 1.00 | 0.00 | N |
| ATOM | 9171 | C7 | NAG | C 1 | 60.821 | 46.670 | 14.171 | 1.00 | 0.00 | C |
| ATOM | 9172 | O7 | NAG | C 1 | 60.320 | 47.760 | 13.899 | 1.00 | 0.00 | O |
| ATOM | 9173 | C8 | NAG | C 1 | 61.708 | 46.528 | 15.397 | 1.00 | 0.00 | C |
| ATOM | 9174 | C3 | NAG | C 1 | 59.957 | 43.237 | 13.482 | 1.00 | 0.00 | C |
| ATOM | 9175 | O3 | NAG | C 1 | 61.007 | 42.887 | 14.371 | 1.00 | 0.00 | O |
| ATOM | 9176 | C4 | NAG | C 1 | 58.775 | 42.293 | 13.686 | 1.00 | 0.00 | C |
| ATOM | 9177 | O4 | NAG | C 1 | 59.153 | 40.971 | 13.331 | 1.00 | 0.00 | O |
| ATOM | 9178 | C5 | NAG | C 1 | 57.593 | 42.746 | 12.824 | 1.00 | 0.00 | C |
| ATOM | 9179 | O5 | NAG | C 1 | 57.240 | 44.112 | 13.139 | 1.00 | 0.00 | O |
| ATOM | 9180 | C6 | NAG | C 1 | 56.355 | 41.902 | 13.057 | 1.00 | 0.00 | C |
| ATOM | 9181 | O6 | NAG | C 1 | 56.115 | 41.030 | 11.963 | 1.00 | 0.00 | O |
| ATOM | 9182 | C1 | SWA | S 1 | 31.083 | 66.852 | 6.104 | 1.00 | 0.00 | C |
| ATOM | 9183 | O1 | SWA | S 1 | 31.616 | 67.993 | 5.412 | 1.00 | 0.00 | O |
| ATOM | 9184 | C3 | SWA | S 1 | 31.290 | 67.004 | 7.648 | 1.00 | 0.00 | C |
| ATOM | 9185 | N4 | SWA | S 1 | 30.728 | 65.809 | 8.307 | 1.00 | 0.00 | N |
| ATOM | 9186 | C5 | SWA | S 1 | 29.253 | 65.674 | 8.135 | 1.00 | 0.00 | C |
| ATOM | 9187 | C6 | SWA | S 1 | 28.949 | 65.502 | 6.622 | 1.00 | 0.00 | C |
| ATOM | 9188 | C2 | SWA | S 1 | 29.552 | 66.691 | 5.818 | 1.00 | 0.00 | C |
| ATOM | 9189 | C9 | SWA | S 1 | 31.281 | 65.713 | 9.651 | 1.00 | 0.00 | C |
| ATOM | 9190 | C8 | SWA | S 1 | 32.647 | 66.446 | 9.597 | 1.00 | 0.00 | C |
| ATOM | 9191 | O13 | SWA | S 1 | 33.714 | 65.532 | 9.832 | 1.00 | 0.00 | O |
| ATOM | 9192 | C7 | SWA | S 1 | 32.754 | 67.081 | 8.161 | 1.00 | 0.00 | C |
| ATOM | 9193 | O11 | SWA | S 1 | 33.638 | 66.311 | 7.317 | 1.00 | 0.00 | O |
| ATOM | 9194 | C1 | MPD | M 1 | 14.801 | 61.371 | 10.217 | 1.00 | 0.00 | C |
| ATOM | 9195 | C2 | MPD | M 1 | 16.246 | 61.411 | 10.589 | 1.00 | 0.00 | C |
| ATOM | 9196 | O2 | MPD | M 1 | 16.899 | 60.292 | 9.952 | 1.00 | 0.00 | O |
| ATOM | 9197 | CM | MPD | M 1 | 16.897 | 62.682 | 10.105 | 1.00 | 0.00 | C |
| ATOM | 9198 | C3 | MPD | M 1 | 16.386 | 61.237 | 12.121 | 1.00 | 0.00 | C |
| ATOM | 9199 | C4 | MPD | M 1 | 17.772 | 60.909 | 12.678 | 1.00 | 0.00 | C |
| ATOM | 9200 | O4 | MPD | M 1 | 17.676 | 59.909 | 13.666 | 1.00 | 0.00 | O |
| ATOM | 9201 | C5 | MPD | M 1 | 18.376 | 62.135 | 13.353 | 1.00 | 0.00 | C |
| ATOM | 9202 | ZN | ZN1 | M 1 | 34.563 | 64.336 | 8.063 | 1.00 | 0.00 | Zn |

TABLE 9
Data Collection Statistics

| | | | | Native dGMII | | |
|---|---|---|---|---|---|---|
| | MAD (Se-Met) of dGMII | | | High | DMNJ | swainsonine |
| | inflection | peak | Remote | resolution | complex | complex |
| Wavelength (Å) | 0.9790 | 0.9786 | 0.9770 | 1.0 | 1.0 | 1.54189 |
| Effective | 2.14 | 2.14 | 2.14 | 1.76 | 1.69 | 1.87 |
| resolution (Å) | | | | | | |
| Maximum | 1.70 | 1.70 | 1.70 | 1.4 | 1.5 | 1.87 |
| resolution (Å) | | | | | | |
| Highest resolution | 2.31-2.14 | 2.31-2.14 | 2.31-2.14 | 1.90-1.76 | 1.75-1.69 | 1.91-1.87 |
| shell | | | | | | |
| Temperature (K.) | 100 | 100 | 100 | 100 | 100 | 100 |
| # unique reflections | | | | | | |
| overall | 59212 | 59092 | 59218 | 104565 | 114653 | 87386 |
| shell | 11288 | 11297 | 11296 | 19882 | 10722 | 5601 |
| completeness (%) | | | | | | |
| overall | 99.7 | 99.8 | 99.8 | 97.0 | 97.8 | 99.7 |
| shell | 96.2 | 96.3 | 96.2 | 94.9 | 92.6 | 96.9 |
| R_{merge*} | | | | | | |
| overall | 0.050 | 0.054 | 0.057 | 0.056 | 0.086 | 0.078 |
| shell | 0.086 | 0.093 | 0.105 | 0.127 | 0.186 | 0.452 |

*R_{merge} = ΣₕΣᵢ|Iᵢ − <I>|/ΣᵢI_{I}, where <I> is the average of equivalent reflections and the sum is extended over all observations, i, for all unique reflections, h.

TABLE 10
Refinement Statistics

| | | dGMII- | |
|---|---|---|---|
| | | swainsonine | dGMII-DMNJ |
| | dGMII | complex | complex |
| Resolution (Å) | 500-1.40 | 500-1.87 | 500-1.5 |
| R_{cryst} (%) | 19.30 | 18.10 | 19.69 |
| R_{free} (%) | 21.05 | 20.90 | 21.56 |
| Atoms (#) | 9194 | 9202 | 9199 |
| Residues (#) | 1014 | 1014 | 1014 |
| Water molecules (#) | 981 | 985 | 983 |
| r.m.s.d. Bonds (Å) | 0.005 | 0.005 | 0.006 |
| r.m.s.d. Angles (°) | 1.32 | 1.31 | 1.33 |
| r.m.s.d. Improper dihedrals (°) | 0.81 | 0.78 | 0.80 |
| Average B-factors (Å²) | 15.8 | 19.4 | 15.8 |
| Crossvalidated | | | |
| σ_{A} coordinate error (Å) | 0.10 | 0.14 | 0.11 |

R_{cryst} = Σ||Fₒ| − |F_{c}||Σ|Fₒ|, where Fₒ and F_{c} are the observed and calculated structure factors, respectively. For R_{free}, the sum is extended over a subset of reflections (˜10%) excluded from all stages of refinement.

### REFERENCES

Alberts, I. L., Nadassy, K., and Wodak, S. J. (1998). Analysis of zinc binding sites in protein crystal structures. Protein Sci. 7, 1700-1716.

Altmann F, Marz L. (1995). Processing of asparagine-linked oligosaccharides in insect cells: evidence for alpha-mannosidase II. Glycoconj. J. 12, 150-155.

Boraston, A., Chiu, P., Warren, R. A. J., and Kilburn, D. G. (2000). Specificity and affinity of substrate binding by a family 17 carbohydrate-binding module from Clostridium cellulovorans Cellulase 5A. Biochemistry 39, 11129-11136.

Braun, C., Brayer, G. D., and Withers, S. G. (1995). Mechanism-based inhibition of yeast alpha-glucosidase and human pancreatic alpha-amylase by a new class of inhibitors. 2-Deoxy-2,2-difluoro-alpha-glycosidases. J. Biol. Chem. 270, 26778-26781.

Brünger A. T., Adams P. D., Clore G. M., DeLano W. L., Gros P., Grosse-Kunstleve R. W., Jiang J. S., Kuszewski J., Nilges M., Pannu N. S., Read R. J., Rice L. M., Simonson T., Warren G. L. (1998). Crystallography & NMR system: A new software suite for macromolecular structure determination. Acta Crystallogr D Biol. Crystallogr. 54, 905-921.

Cowtan, K. (1994). ‘dm’: an automated procedure for phase improvement by density modification. CCP4 ESF-EACBM Newslett. Protein Crystallogr. 31, 34-38.

Coutinho, P. M. and Henrissat, B. (1999). Carbohydrate-Active Enzymes server at URL: http://afmb.cnrs-mrs.fr/˜pedro/CAZY/db.html

Davies, G., and Henrissat, B. (1995). Structures and mechanisms of glycosyl hydrolases. Structure 3, 853-859.

Dennis, J. W., and Laferte, S. (1985). Recognition of asparagine-linked oligosaccharides on murine tumor cells by natural killer cells. Cancer Res. 45, 6034-6040.

Dennis, J. W., Granovsky, M., and Warren, C. E. (1999a) Protein glycosylation in development and disease. Bioessays 21, 412-421.

Dennis, J. W., Granovsky, M., and Warren, C. E. (1999b) Glycoprotein glycosylation and cancer progression. Biochim. Biophys. Acta 1473, 21-34.

Goss, P. E., Baker, M. A., Carver, J. P., and Dennis, J. W. (1995). Inhibitors of carbohydrate processing, A new class of anticancer agents. Clin. Cancer Res. 1, 935-944.

Goss, P. E., Reid, C. L., Bailey, D., and Dennis, J. W. (1997). Phase IB clinical trial of the oligosaccharide processing inhibitor swainsonine in patients with advanced malignancies. Clin. Cancer Res. 3, 1077-1086.

Harpaz, N. and Schachter, H. (1980). Control of Glycoprotein Synthesis. V. Processing of asparagine-linked oligosaccharides by Golgi alpha-D-mannosidases dependent on the prior action of UDP-N-acetylglucosamine:alpha-D-mannoside beta-2-N-acetylglucosaminyltransferase I. J. Biol. Chem. 255, 4894-4902.

Henrissat, B. (1991). A classification of glycosyl hydrolases based on amino-acid sequence similarities. Biochem. J. 280, 309-316.

Herscovics, A. (1999). Importance of glycosidases in mammalian glycoprotein biosynthesis. Biochim. Biophys. Acta 1473, 96-107.

Howard, S., He, S., and Withers, S. G. (1998). Identification of the active site nucleophile in Jack-bean alpha-mannosidase using 5-fluoro-beta-L-gulosyl fluoride. J. Biol. Chem. 273, 2067-2072.

Jones, T A, Zou, J. Y., Cowan, S. W., and Kjeldgaard, M. (1991). Improved methods for building protein models in electron density maps and the location of errors in these models. Acta Crystallogr. A 47, 110-119.

Kausal, G. P., Szumilo, T., Pastuszak, I., and Elbein, A. D. (1990). Purification to homogeneity and properties of mannosidase II from mung bean seedlings. Biochemistry 29, 2168-2176.

Kiyohara, T., Dennis, J. W., and Roder, J. C. (1987). Double restriction in NK cell recognition is linked to transmethylation and can be triggered by asparagine-linked oligosaccharides on tumor cells. Cell. Immunol. 106, 223-233.

Kornfeld R., and Kornfeld S. (1985). Assembly of asparagine-linked oligosaccharides. Annu. Rev. Biochem. 54, 631-664.

Kraulis, P. (1991). Molscript: a program to produce both detailed and schematic plots of protein structures. J. Appl. Crystalogr. 24, 946-950.

Merritt, E. A., and Bacon, D. J. (1997). Raster3D: photorealistic molecular graphics. Methods Enzymol. 277, 505-524.

Moremen K. W., Trimble R. B., Herscovics A. (1994). Glycosidases of the asparagine-linked oligosaccharide processing pathway. Glycobiology. 4, 113-125.

Moremen, K. W. and Touster O. (1985). Biosynthesis and modification of Golgi mannosidase II in HeLa and 3T3 cells. J. Biol. Chem. 260, 6654-6662.

Moremen, K. W. and Touster O. (1986). Topology of mannosidase II in rat liver Golgi membranes and release of the catalytic domain by selective proteolysis. J. Biol. Chem. 261, 10945-10951.

Nicholls A., Sharp K. A., Honig B. (1991). Protein folding and association: insights from the interfacial and thermodynamic properties of hydrocarbons. Proteins 11 281-296.

Rabouille, C., Kuntz, D. A., Lockyer, A., Watson, R., Signorelli, T., Rose, D. R., van den Heuvel,. M., and Roberts, D. B. (1999) The Drosophila GMII gene encodes a Golgi α-mannosidase II. J. Cell Sci. 112, 3319-3330.

Ren, J., Castellino, F. J., and Bretthauer, R. K. (1997). Purification and properties of □-mannosidase II from Golgi-like membranes of baculovirus-infected Spodoptera frugiperda (IPLB-SF-21AE) cells. Biochem. J. 324, 951-956.

Schmidt, A., Schlacher, A., Steiner, W., Schwab, H., and Kratky, C. (1998). Structure of xylanase from Penicillium simplicissimum. Protein Sci. 7(10), 2081-8

Snaith, S. M. (1975) Characterization of jack-bean alpha-D-mannosidase as a zinc metalloenzyme. Biochem. J. 147, 83-90.

Terwilliger T. C., Kim, S. H., and Eisenberg J. (1987). Generalized method of determining heavy-atom positions using the difference Patterson function. Acta Crystallogr. A, 43, 1-5. Vallée, F., Lipari, F., Yip, P., Sleno, B., Herscovics, A., and Howell, P. L. (2000). Crystal structure of a class I □1,2-mannosidase involved in N-glycan processing and endoplasmatic reticulum quality control. EMBO J. 19, 581-588.

Wallace A. C., Laskowski R. A., Thornton J. M. (1995). LIGPLOT: a program to generate schematic diagrams of protein-ligand interactions. Protein Eng. 8, 127-134.

White, A., and Rose, D. R. (1997). Mechanism of catalysis by retaining b-glycosyl hydrolases. Curr. Opin. Struct. Biol. 7, 645-651.

1. A crystal comprising a mannosidase II ligand-binding domain.

2. A crystal according to claim 1, which is a crystal of a mannosidase II.

3. A crystal according to claim 2 characterized by an N-terminal α/β domain, a C-terminal portion comprising a three-helical bundle, and an all-β C-terminal domain, connected by 5 internal disulfide bonds and stabilized by a zinc binding site.

4. A crystal according to claim 3 wherein the N-terminal α/β domain is characterized by the following:
(a) comprising an inner core of three β-sheets (A, B and C, FIG. 8B) consisting of 11, mostly parallel β-strands, surrounded by 16 α-helices;
(b) comprising a GlcNAc residue at a consensus N-glycosylation site (Asn-194), located at the N-terminus of helix 7; and
(c) stabilized by three disulfide bonds: between Cys-31 and Cys-1032 connecting the N and C-terminal extremes of dGMII; Cys-275 and Cys-282 linking helices 10 and 11; Cys-283 and Cys-297 linking helix 11 with a loop between helix 13 and the core of parallel β-sheets.

5. A crystal according to claim 3 wherein the C-terminal portion is characterized by the following:
(a) a three-helix bundle comprises helices 18, 20 and 21 connected to the N-terminal α/β-domain via a zinc binding site;
(b) a zinc ion coordinated in a T₅-square-based pyramidal geometry involving residues: Asp-90, His-92, Asp-204 and His-471;
(c) two immunoglobulin-like domains: a small β-sandwich consisting of 12 anti-parallel strands from β-sheets D and E, and a large 21 -strand structure involving β-sheets F and G; and
(d) a barrel formed by the three-helix bundle, helix-23, and the two β-sandwich structures provides a narrow pore in the center of the C-terminal domain.

6. A crystal according to claim 1 or 2, comprising a complex between a mannosidase II ligand-binding domain and at least one ligand.

7. A crystal according to claim 3, wherein the ligand is swainsonine or a derivative thereof.

8. A crystal as claimed in claim 2 which is characterized by the following:
(a) a small cavity lined by aromatic residues Trp-95, Phe-206, Tyr-269 and Tyr-727;
(b) a zinc ion binding site within the cavity characterized by a Ts-square-based pyramidal geometry and ‘elec-His-Zn motifs’.

9. A crystal as claimed in claim 1 wherein the ligand binding domain comprises one or more of amino acid residues Trp-95, Phe-206 and Tyr-727 which form a binding cavity for a mannosidase II inhibitor.

10. A crystal as claimed in claim 1 wherein the ligand binding domain is capable of binding a zinc ion characterized by a Ts-square-based pyramidal geometry involving amino acid residues: Asp-90, His-92, Asp-204 and His-471

11. A crystal as claimed in claim 1 wherein the ligand binding domain comprises one or more of amino acid residues: His 471, His 90, and Asp 92, and Asp 204; or a homologue thereof

12. A crystal as claimed in claim 1 wherein the ligand binding domain comprises one or more of amino acid residues: Trp-95, Phe-206, Tyr-269, and Tyr-727.

13. A crystal as claimed in claim 1 wherein the ligand binding domain comprises one or more of amino acid residues: Asp-92, Asp-204, His-90, His-471.

14. A crystal according to claim 1 wherein said ligand-binding domain comprises one or more of the following residues: His 471, Asp 204, Asp 341, His 90, Asp 92, Asp 472, Phe 206, Tyr 727 and Tyr 95.

15. A crystal according to claim 1 which comprises one or more of the residues shown in Table 3 or 4.

16. A crystal according to claim 1 wherein said ligand-binding domain comprises one or more of the following groups:

| | |
|---|---|
| (a) | GVWKQG (residues 60-65) |
| (b) | VFVVPHSHND (residues 83-92) |
| (c) | WAIDPFGH (residues 201-208) |
| (d) | HMMPFYSYDIPHTCGPDPKV/ICCQFDFKR (residues 262-289) |
| (e) | LLI/APLGDDFR (residues 334-343). |

17. A crystal according to any preceding claim, wherein the crystal has P2₁ symmetry.

18. A crystal according to any preceding claim, wherein said crystal comprises a unit cell having the following dimensions: a=69 (±5) Å, b=110 (±5) Å, c=139 (±5) Å.

19. A crystal according to any preceding claim having the structural coordinates as shown in Table 1, Table 2, or Table 8.

20. A crystal according to claim 2 comprising one or more of a cofactor, a mannosidase II inhibitor, or a substrate.

21. A crystal of a mannosidase II according to claim 2 defined by the interactions of Table 4.

22. A crystal comprising swainsonine or a derivative thereof having the structural coordinates as shown in Table 2 or Table 8.

23. A computer readable medium having stored thereon: the structure of a crystal according to any of claims 1 to 21.

24. Machine readable media encoded with data representing the structural coordinates of a crystal or ligand binding domain according to any of the preceding claims.

25. A method of screening for a ligand capable of binding a mannosidase II ligand binding domain, comprising the use of a crystal according to any of claims 1 to 21.

26. A method of screening for a ligand according to claim 25, which comprises the step of contacting the ligand binding domain with a test compound, and determining if said test compound binds to said ligand binding domain.

27. A ligand identified by a method according to claim 25 or 26.

28. A ligand according to claim 27, which is capable of interacting with one or more of the residues of a mannosidase II shown in Table 3 or 4.

29. A modulator of the activity of a mannosidase II derived from a crystal as claimed in any of the preceding claims.

30. A method for identifying a potential modulator of a mannosidase II, or ligand binding domain thereof, comprising the step of using the structural coordinates of Table 1, 2, or 8 that define a mannosidase II or ligand binding domain thereof, to computationally evaluate a test compound for its ability to associate with the mannosidase II or ligand binding domain, wherein a test compound that associates is a potential modulator of a mannosidase II.

31. A method for identifying a modulator of a mannosidase II by determining binding interactions between a test compound and binding site of a ligand binding domain of a mannosidase II as defined in Table 4 comprising:
(a) generating the binding site on a computer screen;
(b) generating a test compound with its spatial structure on the computer screen; and
(c) testing to determine whether the test compound binds to a selected number of atomic contacts in a binding site.

32. A method for identifying a potential modulator of a mannosidase II function comprising the steps:
(a) docking a computer representation of a test compound from a computer data base with a computer representation of a crystal of a mannosidase II as claimed in the preceding claims, to obtain complexes;
(b) determining conformations of complexes with a favourable geometric fit and favourable complementary interactions; and
(c) identifying a conformation of a compound that best fits the selected site as a potential modulators of the mannosidase II.

33. A method for identifying a potential modulator of a mannosidase II function comprising the steps:
(a) modifying a computer representation of a test compound complexed with a crystal of a ligand binding domain of a mannosidase II as described in any of the preceding claims, by deleting or adding a chemical group or groups;
(b) determining a conformation of the complex with a favourable geometric fit and favourable complementary interactions; and
(c) identifying a compound that best fits the binding site as a potential modulator of a mannosidase II.

34. A method for identifying a potential modulator of a mannosidase II function co comprising the steps:
(a) selecting a computer representation of a test compound complexed with a crystal of a ligand binding domain of a mannosidase II as defined in the preceding claims; and
(b) searching for molecules in a data base that are similar to the test compound using a searching computer program, or replacing portions of the test compound with similar chemical structures from a data base using a compound building computer program.

35. A modulator of a mannosidase II identified by a method according to any of the preceding claims.

36. A modulator of a mannosidase II based on the three-dimensional structure of an inhibitor's spatial association with a crystal as claimed in any of the preceding claims.

37. A method for designing potential inhibitors of a mannosidase II comprising the step of using the structural coordinates of a mannosidase II inhibitor defined in relation to its spatial association with a crystal of a mannosidase II or a ligand binding domain thereof according to any of the preceding claims, to generate a compound that is capable of associating with the mannosidase II or ligand binding domain thereof.

38. The use of a ligand according to claim 27 or 28, in the manufacture of a medicament to treat and/or prevent a disease in a mammalian patient.

39. A pharmaceutical composition comprising a ligand according to any of claims 27 or 28 and optionally a pharmaceutically acceptable carrier, diluent, excipient or adjuvant or any combination thereof.

40. A pharmaceutical composition comprising a modulator according to any of the preceding claims either alone or with other active substances.

41. A method of treating a disease associated with a mannosidase II in a cellular organism, comprising:
(a) administering a pharmaceutical composition according to claim 39 or 40; and
(b) activating or inhibiting a mannosidase II to treat the disease.

42. A method of treating and/or preventing a disease comprising administering a ligand according to claim 27 or 28 and/or a pharmaceutical composition according to claim 39 or 40 to a mammalian patient.

43. A method of determining the secondary and/or tertiary structures of a polypeptide with unknown structure comprising the step of using a crystal according to any of claims 1 to 21.

44. Plasmid pCopBlast.

45. A host cell comprising a plasmid as claimed in claim 44.

46. A method for preparing a mannosidase II using a plasmid as claimed in claim 44.

47. A method for preparing a mannosidase II is provided comprising:
(a) transferring a plasmid as claimed in claim 44, into a host cell;
(b) selecting transformed host cells from untransformed host cells;
(c) culturing a selected transformed host cell under conditions which allow expression of the mannosidase II and
(d) isolating the mannosidase II.
